(19) United States
(12) Patent Application Publication
(10) Pub. No.: US 2025/0339551 A1
(43) Pub. Date: Nov. 6, 2025

(54) AN ANTIBODY-DRUG CONJUGATE HAVING TWO OR MORE DIFFERENT FUNCTIONAL SMALL MOLECULES FOR ENHANCED TREATMENT OF REFRACTORY DISEASES

(71) Applicants: Robert Yongxin ZHAO, Lexington, MA (US); HANGZHOU SEEHE BIOTECHNOLOGY CO., LTD, Hangzhou (CN); HANGZHOU DAC BIOTECH CO., LTD, Hangzhou (CN)

(72) Inventors: Robert Yongxin ZHAO, Lexington, MA (US); Qingliang YANG, Hangzhou (CN); Yuanyuan HUANG, Hangzhou (CN); Hangbo YE, Hangzhou (CN); Lingli ZHANG, Hangzhou (CN); Huihui GUO, Hangzhou (CN); Junxiang JIA, Hangzhou (CN); Juan WANG, Hangzhou (CN); Wenjun LI, Hangzhou (CN); Lu BAI, Hangzhou (CN)

(73) Assignees: HANGZHOU SEEHE BIOTECHNOLOGY CO., LTD, Hangzhou (CN); HANGZHOU DAC BIOTECH CO., LTD, Hangzhou (CN)

(21) Appl. No.: 19/216,389

(22) Filed: May 22, 2025

Related U.S. Application Data
(63) Continuation-in-part of application No. PCT/CN2023/096066, filed on May 24, 2023.

(51) Int. Cl. A61K 47/68; A61K 45/06; A61P 35/00

(52) U.S. Cl. CPC A61K 47/68037; A61K 45/06; A61K 47/68031; A61K 47/68033; A61K 47/68035; A61K 47/6809; A61K 47/6831; A61K 47/6889; A61P 35/00

(57) ABSTRACT

The present invention relates to an antibody-drug conjugate containing two or more functional small molecules for enhancement of targeted treatment of cancers and refractory diseases. The invention also relates to preparation of such conjugate, pharmaceutical compositions, and methods in treatment of cancers and refractory diseases.

### INFORMATION OF PRIORITY

The present application claims the benefit of PCT/CN2024/094651 field on May 22, 2024, which is incorporated herein reference.

### BACKGROUND

Antibody-drug conjugates (ADCs) that combine the target specificity of a monoclonal antibody (mAb) with the potent cytotoxic drugs are a rapidly expanding class of therapeutic agents against cancers. There are 15 ADCs so far approved as monotherapy or combinational therapy for several solid and liquid cancers. Due to the spectacular success of 9 of the 15 ADC approved in the past 5 years for some highly treatment-refractory diseases, over a 200 new ADCs are now in clinical trials encompassing a wide variety of tumor types according to https://www.clinicaltrials.gov. Despite the explosion of interest in ADCs, challenges remain to expand their therapeutic index (with greater efficacy and less toxicity) and applications in treatment of various cancers (Dean, A. Q., et al, mAbs, 2021, 13 (1), 1951427). Thus, more and more ADCs are combined with chemotherapeutical drugs, immunotherapy compounds and others for the expansion in the treatment (Li, Y, et al, Am J Cancer Res. 2023, 13 (1): 161-17; Ceci, C., et al, Pharmacol. 2022, 236:108106; Gerber, H. P, et al, Biochem Pharmacol. 2016, 102:1-6). However, the half-lives of chemotherapeutical drugs are normally quite short and they have to be given more frequently to patients, therefore leading to more toxicity in synergistic use with ADCs (in comparison with application of the monotherapy of ADCs) (Fuentes-Antrás, J., et al, Trends Cancer 2023, 9 (4): 339-354). Here in this application, we disclose an ADC containing one or more chemical drugs or functional small molecules covalently linked to the antibody, which can expand the half-lives of the conjugated small molecules, resulting in enhancement of the targeted treatment of various cancers and refractory diseases. Further disclosed are preparation of the conjugate, pharmaceutical compositions, screening, and medical treatment methods.

### BRIEF SUMMARY OF THE INVENTION

The present invention provides an antibody-drug conjugate (ADC) with a branch linked chemical drugs or functional small molecules for the enhancement of targeted treatment of cancers and refractory diseases. The preferred formulas of the ADCs are represented as (I), (II), (III) and (IV) below:

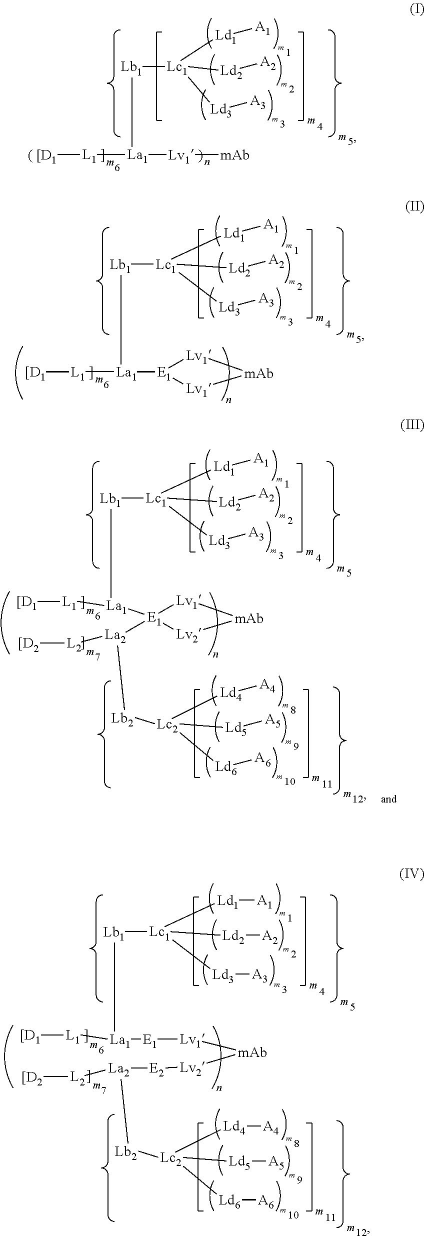

Wherein,
D₁ and D₂ are a cytotoxic agent; mAb is an antibody, an antibody like protein or a cell-binding protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker, which are independently selected from O, C, CH, CH₂, NH, S, N, NH—NH, N—N, N(R₃), N(R₃)N(R_{3′}), C(═O), C(═O)N, C(═O)NH, C(═O)N—N, C₂-C₈ of alkyl (alkylene), heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or 1˜8 natural or unnatural amino acids described in the definition; or polyethyleneoxy unit of formula (OCH₂CH₂) POR₃, (OCH₂CH₂)ₚ, (OCH₂CH(CH₃))ₚOR₃, NH(CH₂CH₂O)ₚR₃, NH(CH₂CH(CH₃)O)ₚR₃, or N[(CH₂CH₂O)ₚR₃][(CH₂CH₂O)ₚR_{3′}], (OCH₂CH₂)ₚCOOR₃, CH₂CH₂ (OCH₂CH₂)ₚCOR₃, or CH₂CH₂ (OCH₂CH₂)ₚCOOR₃, wherein p and p′ are independently an integer selected from 0 to about 100, or combination thereof; wherein R₃ and R_{3′} are independently H, C(═O) H, C(═O)CH₃, NH, C(═O), C(═O)NH, C₁-C₈ of alkyl, absent; or combination above thereof; and one or several of L₁, L₂, La₁, La₂, Lb₁, Lb₂, Lc₁, Lc₂, Ld₁, Ld₂, Ld₃, Ld₄, Ld₅, and Ld₆ can be absent thereof;
In addition, La₁ and La₂ are independently a peptide having 1˜ 8 amino acids, which contain one or several lysine, glutamic acid, aspartic acid, cysteine, glutamine, asparagine and tyrosine, and these amino acids that link A₁, A₂, A₃, A₄, A₅ and A₆ through Lb₁, Lb₂, Lc₁, Lc₂, Ld₁, Ld₂, Ld₃, Ld₄, Ld₅, and Ld₆ accordingly;
E₁ and E₂ are a joint group that link two reactive groups, Lv₁ and Lv₂, which preferably can react to a thiol, amino, phenol, ketone, aldehyde, alkyne, hydroxyl, or carboxylic acid group in an antibody to form a group of Lv₁′ and Lv₂′ respectively. E₁ and E₂ are independently selected from CH, CH₂, CH—CH, NH, NHNH, N(R₃), N(R₃)N(R_{3′}), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1˜4 units of amino acids, preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine; more detail structures are described in the specification of the invention. In addition, E₁ can be absent, thus La₁ or/and La₂ can directly link to a group of Lv₁′ or Lv₂′;
m₁, m₂, m₃, m₄, m₅, m₆, m₇, m₈, m₉, m₁₀, m₁₁ and m₁₂ are independently 1, 2, 3, 4, 5, 6, 7, 8, 9, or 10 and these numbers may have a decimal; in addition, m₂, m₃, m₈, m₉ and/or m₁₀ can be 0, thus Ld₂-A₂, Ld₃-A₃, Ld₅-A₅, and/or Ld₆-A₆ can be absent;
A₁, A₂, A₃, A₄, A₅ and A₆ are a functional small molecule, independently selected from an affinity ligand such as for bombesin receptors/neurotensin receptors (including neuropeptide-Y receptors), a cell-penetrating peptide; a nucleoside antimetabolite/analog, or/and a chemotherapy drug; which have either the synergy with D₁ and/or D₂, or enhanced affinity for mAb. The detail structures are described in the specification of the invention.
wherein Lv₁′ and Lv₂′ are a group that are resulted from reaction of Lv₁ and Lv₂ to an amino acid of an antibody or a cell-binding protein independently. The detail structures of Lv₁′ and Lv₂′ are described in the specification of the invention.

The present invention also provides an antibody-drug conjugate (ADC), comprising a monoclonal antibody, or an antigen-binding fragment thereof, a cytotoxin, and a linker having a functional small molecule, and/or an affinity ligand, such as for bombesin receptors/neurotensin receptors (including neuropeptide-Y receptors), and/or a cell-penetrating peptide, and or an affinity peptide, such as the programmed death ligand-1 (PD-L1, or CD274), expressed on tumor cells and tumor-infiltrating immune cells, resulting in enhancement of targeted treatment of cancers and refractory diseases. In a further embodiment the antigen binding proteins are conjugated to a potent toxin such as a tubulysin analog, a camptothecin (CPT) analog, a PBD dimer, an eribulin, an auristatin analog, a duocarmycin analog, or an anthracycline analog, or the other cytotoxic agent or its analogs that described in the present invention.

In addition, the invention provides compositions comprising the foregoing antibody-drug conjugate, and a pharmaceutically acceptable carrier, and methods of the targeted treatment of various cancers and refractory diseases.

### BRIEF DESCRIPTION OF THE SEVERAL VIEWS OF THE DRAWING(S)

FIG. 1 shows the Extracted Ion Chromatograms (EIC) and MS/MS Spectra of the Drug-Conjugated Peptide [SC₍₂₂₇₎DK] in Steap1-C002 ADC_GLP DP_2401B101 batch (signature fragments of Steap1-C002 ADC at DAR=4.0). Figure a is Extracted Ion Chromatogram (EIC) of the precursor ion of the drug-conjugated peptide; Figures b and c are the MS/MS spectrum of the drug-conjugated peptide at a retention time (RT) of 53.28 min, and the zoomed-in MS/MS spectrum in the m/z range of 140 to 810, respectively.

FIG. 2 shows The Extracted Ion Chromatograms (EIC) and MS/MS Spectra of the Drug-Conjugated Peptide [GEC₍₂₂₀₎] in Steap1-C002 ADC_GLP DP_2401B101 batch (signature fragments of Steap1-C002 ADC at DAR=4.0). Figure a is Extracted Ion Chromatogram (EIC) of the precursor ion of the drug-conjugated peptide; Figures b and c are the MS/MS spectrum of the drug-conjugated peptide at a retention time (RT) of 58.34 min, and the zoomed-in MS/MS spectrum in the m/z range of 140 to 800, respectively.

FIG. 3 shows The Extracted Ion Chromatograms (EIC) and MS/MS Spectra of the Drug-Conjugated Peptide [SC₍₂₂₇₎DK] in Steap1-C002 ADC 008B24016-240418, (signature fragments of Steap1-C002 ADC at DAR-6.0). Figure a is Extracted Ion Chromatogram (EIC) of the precursor ion of the drug-conjugated peptide; Figures b and c are the MS/MS spectrum of the drug-conjugated peptide at a retention time (RT) of 53.28 min, and the zoomed-in MS/MS spectrum in the m/z range of 140 to 810, respectively

FIG. 4 shows The Extracted Ion Chromatograms (EIC) and MS/MS Spectra of the Drug-Conjugated Peptide [GEC (220)] in Steap1-C002 ADC-008B24016-240418 (signature fragments of Steap1-C002 ADC at DAR=6.0). Figure a is Extracted Ion Chromatogram (EIC) of the precursor ion of the drug-conjugated peptide; Figures b and c are the MS/MS spectrum of the drug-conjugated peptide at a retention time (RT) of 58.97 min, and the zoomed-in MS/MS spectrum in the m/z range of 140 to 800, respectively.

FIG. 5 shows the HIC-HPLC analysis of the Steap1-C002 conjugate at DAR=4.0 by the specific conjugation of the present invention, wherein the distributions of Ds (drugs/antibody) were D0=2.02%, D2=5.58%, D4=86.78%, D6=2.91% and D8=2.71%; and D4 was over 85%.

FIG. 6 shows the HIC-HPLC analysis of the Steap1-C002 conjugate at DAR=6.0 by the specific conjugation of the present invention, wherein the distributions of Ds (drugs/antibody) were D0=0.00%, D2=0.81%, D4=8.78%, D6=80.90% and D8=9.51%; and D6 was over 80%.

FIG. 7 shows the affinities of the prepared vandortuzumab, vandortuzumab containing LALA sequence (generated in house), a more soluble vandortuzumab-1 (Steap1 mAb1 generated in house), vandortuzumab-C002 ADC and an isotype (an anti-HIV antibody) against C4-2B cells. Since C4-2B cells highly express both Steap1 and PSMA antigens, thus the vandortuzumab-C002, which only contained PSMA binders (DUPA function groups), demonstrated better affinity for the C4-2B cells than the naked (unconjugated) antibodies. It also demonstrated that the prepared vandortuzumab and vandortuzumab containing LALA sequence had the similar affinities and the prepared vandortuzumab-1 (Steap1 mAb-1) had a little bit lower affinity than the its original format. The isotype antibody has no affinity at all against C4-2B cells.

FIG. 8 shows the affinities of the prepared vandortuzumab, vandortuzumab-C002 ADC and an isotype (an anti-HIV antibody) against PC-3-4B9 cells. Since PC-3-4B9 only express highly PSMA antigens, thus the vandortuzumab-C002, which only contained PSMA binders (DUPA function groups) had the affinity for PC-3-4B9 cells. In contrast, both vandortuzumab and the isotype antibodies had no PSMA binders (no DUPA groups) and therefore could not show the affinity.

FIG. 9 shows the affinities of the prepared vandortuzumab, vandortuzumab-C002 ADC and an isotype (an anti-HIV antibody) against PC-3-4G5 cells. Since PC-3-4G5 only express highly Steap1 antigens, thus both prepared vandortuzumab and the vandortuzumab-C002 had the good affinity for PC-3-4G5 cells. The isotype antibody had no Steap1 binders and therefore was not able to show the affinity.

FIG. 10 shows the affinities of the prepared vandortuzumab, vandortuzumab-C002 ADC and an isotype (an anti-HIV antibody) against nature PC-3 cells. Since PC-3 cells do not have either Steap1 or PSMA antigens, thus all tested vandortuzumab, the vandortuzumab-C002 and the isotype antibody were not able to show the affinity at all.

FIG. 11 shows in vivo activity over time with one time administration of ADCs along with Steap1-GGFG-Dxd at 6.0 mg/Kg dose over 60 days. It demonstrated that the conjugates containing the payload/linker complexes of the present application had better antitumor activity than the regular GGFG-Dxd conjugate.

FIG. 12 shows body weight changes in CD-1 mice over time with one time administration of ADCs along with Steap1-GGFG-Dxd at 400 mg/Kg dose over 12 days. It demonstrated that the tested conjugates containing the payload/linker complexes of the present application had less side toxicity than the regular GGFG-Dxd conjugate.

FIG. 13 shows in vivo activity over time with one time administration of anti-AXL antibody drug conjugates (312, 328, 314, 320, 262, 259, 339, 293, 360, 368, 275, 353) along with AXL-GGFG-Dxd (all of them with DAR=4.0˜4.2) at 6.0 mg/Kg dose over 64 days against MDA-MB-231 human breast cancer cells. It demonstrated that the conjugates containing both topoisomerase inhibitor (CPT) compound and gemcitabine compound of the present application had better antitumor activity than the regular GGFG-Dxd conjugate.

FIG. 14 shows in vivo activity over time with one time administration of anti TF antibody-drug conjugates (393, 388, 395, 449, 484, 488, 490, 491, 405, 375, 382, 385, 405, 441) along with anti TF-GGFG-Dxd (all of them with DAR=4.0˜4.2) at 6.0 mg/Kg dose over 36 days against BxPC-3 human pancreatic cancer cells. It demonstrated that the conjugates containing both topoisomerase inhibitor (CPT) compound and gemcitabine or peptide ligand compound of the present application had better antitumor activity than the regular GGFG-Dxd conjugate.

FIG. 15 shows in vivo activity over time with one time administration of anti GUCY2C antibody drug conjugates (488, 449, 490, 491, 497, 495, 496, 570, 708, 712, 713, 707) along with anti GUCY2C antibody-GGFG-Dxd conjugate (all of them with DAR=4.0˜4.2) at 3.0 mg/Kg dose over 20 days against GUCY2C-9C9 colon cancer cells. It demonstrated that the conjugates containing both topoisomerase inhibitor (CPT) compound and a gemcitabine or a peptide ligand compound of the present application had better antitumor activity than the regular GGFG-Dxd conjugate.

FIG. 16 shows in vivo activity over time with one time administration of anti 5T5 antibody drug conjugates (507, 497, 501, 500, 503, 502, 504, 505, 506, 509, 508, 510) along with anti 5T4 antibody-GGFG-Dxd conjugate (all of them with DAR=4.0˜4.4) at 5.0 mg/Kg dose over 44 days against NCI-N87 gastric cancer cells. It demonstrated that the conjugates containing both topoisomerase inhibitor (CPT) compound and a peptide ligand compound of the present application had better antitumor activity than the regular GGFG-Dxd conjugate.

FIG. 17 shows in vivo activity over time with one time administration of anti c-Met/PD-L1 bispecific antibody drug conjugates (523, 519, 514, 512, 515, 517, 516, 518, 513, 520, 522, 521) along with anti c-Met/PD-L₁ bispecific antibody-GGFG-Dxd conjugate (all of them with DAR=4.0˜4.3) at 2.0 mg/Kg dose over 39 days against NCI-HCC827 NSCLC cancer cells. It demonstrated that the conjugates containing both topoisomerase inhibitor (CPT) compound and a peptide ligand compound of the present application had better antitumor activity than the regular GGFG-Dxd conjugate.

FIG. 18 shows in vivo activity over time with one time administration of anti EGFR/c-Met bispecific antibody drug conjugates (657, 670, 671, 669, 658, 666, 659, 658, 660, 661, 663, 662) along with anti EGFR/c-Met bispecific antibody-GGFG-Dxd conjugate (all of them with DAR=4.0˜4.2) at 1.5 mg/Kg dose over 23 days against NCI-H1975 Lung cancer cells. It demonstrated that the conjugates containing both topoisomerase inhibitor (CPT) compound and a peptide ligand compound of the present application had better antitumor activity than the regular GGFG-Dxd conjugate.

FIG. 19 shows in vivo activity over time with one time administration of anti Her2 (transtuzumab/pertuzumab) bispecific antibody drug conjugates (108, 55, 41, 106, 76, 134, 128, 126, 142, 91, 66, 172) along with anti Her2 bispecific antibody-GGFG-Dxd conjugate (all of them with DAR=4.0˜4.2) at 2.0 mg/Kg dose over 46 days against JIMT-1 (Dxd resistant) breast cancer cells. It demonstrated that the conjugates containing both topoisomerase inhibitor (CPT) compound and a tubulysin analog compound of the present application had better antitumor activity than the regular GGFG-Dxd conjugate.

FIG. 20 shows in vivo activity over time with one time administration of anti EGFR/Muc-1 bispecific antibody drug conjugates (353, 360, 368, 375, 382, 385, 395, 405, 441, 449, 293, 275) along with anti EGFR/Muc-1 bispecific antibody-GGFG-Dxd conjugate (all of them with DAR=4.0˜4.2) at 4.0 mg/Kg dose over 36 days against CFPAC pancreatic cancer cells. It demonstrated that the conjugates containing both topoisomerase inhibitor (CPT) compound and gemcitabine analog compound of the present application had better antitumor activity than the regular GGFG-Dxd conjugate.

FIG. 21 shows in vivo activity over time with one time administration of anti Her2 bispecific antibody drug conjugates (259, 275, 262, 293, 308, 320, 339, 314, 312, 393, 388, 328) along with anti Her2 bispecific antibody-GGFG-Dxd conjugate (all of them with DAR=4.0˜4.2) at 3.0 mg/Kg dose over 36 days against N87 gastric cancer cells. It demonstrated that almost all of the conjugates except 328 conjugates containing both topoisomerase inhibitor (CPT) compound and gemcitabine analog compound of the present application had better antitumor activity than the regular GGFG-Dxd conjugate.

FIG. 22 shows in vivo activity over time with one time administration of anti Her2 bispecific antibody drug conjugates (508, 512, 497, 488, 574) along with anti Her2 bispecific antibody-GGFG-Dxd conjugate (all of them with DAR=4.0˜4.2) at 5.0 mg/Kg dose over 29 days against CFPAC pancreatic cancer cells. It demonstrated that almost the conjugates containing topoisomerase inhibitor (CPT) compound and a peptide ligand compound of the present application had better antitumor activity than the regular GGFG-Dxd conjugate

FIG. 23 shows in vivo activity over time with one time administration of anti B7H3 antibody drug conjugates (C046, C047, C044, C040, C045) along with anti B7H3 antibody-GGFG-Dxd conjugate (all of them with DAR=4.0˜4.2) at 4.0 mg/Kg dose over 15 days against C4-2B prostate cancer cells. It demonstrated that almost the conjugates containing topoisomerase inhibitor (CPT) compound and a DUPA ligand compound of the present application had better antitumor activity than the regular GGFG-Dxd conjugate.

FIG. 24 shows in vivo activity over time with one time administration of anti B7H3 antibody drug conjugates (C027, C025, C031, C046, C059, C061, C063) along with anti B7H3 antibody-GGFG-Dxd conjugate (all of them with DAR=4.0˜4.2) at 6.0 mg/Kg dose over 11 days against 22RV1 prostate cancer cells. It demonstrated that almost the conjugates containing topoisomerase inhibitor (CPT) compound and a DUPA ligand compound of the present application had better antitumor activity than the regular GGFG-Dxd conjugate.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N3, —NH₂, —NH(R′), —N(R′)₂ and —CN; where each R′ is independently selected from —C₁-C₈alkyl and aryl. When the alkyl group is inserted in the middle of a group, such as in a middle of a linker, thus the “alkyl” means “alkylene” group in this application.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4, 5], [5, 5], [5, 6] or [6, 6] system, or 9 or 10 ring atoms arranged as a bicycle [5, 6] or [6, 6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈alkyl, —O—(C₁-C₈alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N3, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1, 2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoro-methylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DIAD, diisopropylazodicarboxylate; DIBAL-H, diisobutyl-aluminium hydride; DIPEA, diisopropylethylamine; DEPC, diethyl phosphorocyanidate; DMA, N, N-dimethyl acetamide; DMAP, 4-(N, N-dimethylamino)pyridine; DMF, N, N-dimethylformamide; DMSO, dimethylsulfoxide; DTT, dithiothreitol; EDC, 1-(3-dimethylaminopropyl)-3-ethylcarbodiimide hydrochloride; ESI-MS, electrospray mass spectrometry; HATU, O-(7-azabenzotriazol-1-yl)-N, N,N′,N′-tetramethyluronium hexafluorophosphate; HOBt, 1-hydroxybenzotriazole; HPLC, high pressure liquid chromatography; NHS, N-Hydroxysuc-cinimide; MMP, 4-methylmorpholine; PAB, p-aminobenzyl; PBS, phosphate-buffered saline (pH 7.0˜7.5); PEG, polyethylene glycol; SEC, size-exclusion chromatography; TCEP, tris(2-carboxyethyl) phosphine; TFA, trifluoroacetic acid; THF, tetrahydrofuran; Val, valine.

The “amino acid(s)” can be natural and/or unnatural amino acids, preferably alpha-amino acids. Natural amino acids are those encoded by the genetic code, and their names, structures, single-letter or three letter codes are well known in a college text book as: G—Glycine (Gly), P—Proline (Pro), A—Alanine (Ala), V—Valine (Val), L—Leucine (Leu), I—Isoleucine (Ile), M—Methionine (Met), C—Cysteine (Cys), F—Phenylalanine (Phe), Y—Tyrosine (Tyr), W—Tryptophan (Trp), H—Histidine (His), K—Lysine (Lys), R—Arginine (Arg), Q—Glutamine (Gln), N—Asparagine (Asn), E—Glutamic Acid (Glu), D—Aspartic Acid (Asp), S—Serine (Ser), T—Threonine (Thr). The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3, 4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1˜8 amino acids are used in this patent application, amino acid sequence is then preferably a cleavage recognition sequence for a protease. Many cleavage recognition sequences are known in the art. See, e.g., Matayoshi et al. Science 247:954 (1990); Dunn et al. Meth. Enzymol. 241:254 (1994); Seidah et al. Meth. Enzymol. 244:175 (1994); Thornberry, Meth. Enzymol. 244:615 (1994); Weber et al. Meth. Enzymol. 244:595 (1994); Smith et al. Meth. Enzymol. 244:412 (1994); and Bouvier et al. Meth. Enzymol. 248:614 (1995); the disclosures of which are incorporated herein by reference. In particular, the sequence is selected from the group consisting of Val-Cit, Val-Ala, Val-Gln, Val-Lys, Tyr-Arg, Phe-Arg, Tyr-Met, Leu-Gln, Met-Thr, Phe-Gln, Thr-Thr, Val-Thr, Ala-Ala, (D)Val-(D)Gln, Val-Lys(NPr₂), Val-Lys(NMe₂), Val-Lys(NEt₂), Val-Lys(NBu₂), Val-Lys(NBz₂), Val-Ala-Val, Lys-Lys, Ala-Asn-Val, Ala-Val-Lys, Ala-Val-Glu, Val-Leu-Lys, Cit-Cit, Val-Lys, Ala-Ala-Asn, Gly-Gly, Gly-Gly-Gly, Ala-Ala-Ala, Ala-Ala-Ala-Glu, Ala-Val-Arg, Ala-Val-Arg-Arg, Ala-Ala-Arg, Ala-Ala-Arg-Arg, Gly-Gly-Phe-Gly, Lys, Cit, Ser, and Glu. Besides 20 standard L- or 20 D-amino acids, some unusual amino acids with letter codes are listed here: Aminobutyric acid (Abu), Amino-isobutyric acid (Aib), α-Cyclohexyl-alanine (Cha), Citrulline (Cit), Diaminopropionic acid (Dap), Hydroxy-lysine (Hyl), Hydroxy-proline (Hyp), Norleucine (Nle), Norvaline (Nva), Ornithine (O), Penicilamine (Pen), Pyroglutamate (Pyr), Sarcosine (Sar), Statine (Sta). Modified amino acids with single codes have the following examples: Asparagine-EDANS (D-EDANS), Cysteine 3-Nitro-2-pyridinesulfanyl (C-NPys), Glutamic acid-EDANS (E-EDANS), Glycine N-methylated (G-NMe), Leucine N-methylated (L-NMe), Serine phosphorylated (pS), Threonine phosphorylated (pT), Tyrosine phosphorylated (pY), Tyrosine O-methylated (Y-OMe), 3-Nitrotyrosine (Y-NO₂), Tyrosine sulphated (sY), Lysine 5-Carboxyfluorescein (K-5-FAM), Lysine 5-Carboxytetramethylrhodamine (K-5-TAMRA), Lysine acetylated (K-Ac), Lysine biotinylated (K-Biotin), Lysine-DABCYL (K-DABCYL), Lysine-DANSYL (K-DANSYL), Lysine-Dnp (K-Dnp), Lysine-Mca (K-Mca), Lysine methylated (K-Me), Lysine dimethylated (K-Me2), Lysine trimethylated (K-Me3).

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, PA, 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known. An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495, 1975, or may be made by recombinant DNA methods such as described in U.S. Pat. No. 4,816,567. The monoclonal antibodies may also be isolated from phage libraries generated using the techniques described in McCafferty et al., Nature 348:552-554, 1990, for example.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen binding subsequences of antibodies) that contain minimal sequence derived from non-human immunoglobulin. Preferably, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “monovalent antibody” comprises one antigen binding site per molecule (e.g., IgG or Fab). In some instances, a monovalent antibody can have more than one antigen binding sites, but the binding sites are from different antigens.

A “monospecific antibody” comprises two identical antigen binding sites per molecule (e.g. IgG) such that the two binding sites bind identical epitope on the antigen. Thus, they compete with each other on binding to one antigen molecule. Most antibodies found in nature are monospecific. In some instances, a monospecific antibody can also be a monovalent antibody (e.g. Fab).

A “bivalent antibody” comprises two antigen binding sites per molecule (e.g., IgG). In some instances, the two binding sites have the same antigen specificities. However, bivalent antibodies may be bispecific.

A “bispecific” or “dual-specific” is a hybrid antibody having two different antigen binding sites. The two antigen binding sites of a bispecific antibody bind to two different epitopes, which may reside on the same or different protein targets.

A “bifunctional” is antibody is an antibody having identical antigen binding sites (i.e., identical amino acid sequences) in the two arms but each binding site can recognize two different antigens.

A “heteromultimer”, “heteromultimeric complex”, or “heteromultimeric polypeptide” is a molecule comprising at least a first polypeptide and a second polypeptide, wherein the second polypeptide differs in amino acid sequence from the first polypeptide by at least one amino acid residue. The heteromultimer can comprise a “heterodimer” formed by the first and second polypeptide or can form higher order tertiary structures where polypeptides in addition to the first and second polypeptide are present.

A “heterodimer”, “heterodimeric protein”, “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

The “hinge region”, “hinge sequence”, and variations thereof, as used herein, includes the meaning known in the art, which is illustrated in, for example, Janeway et al., ImmunoBiology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

The “immunoglobulin-like hinge region”, “immunoglobulin-like hinge sequence,” and variations thereof, as used herein, refer to the hinge region and hinge sequence of an immunoglobulin-like or an antibody-like molecule (e.g., immunoadhesins). In some embodiments, the immunoglobulin-like hinge region can be from or derived from any IgG1, IgG2, IgG3, or IgG4 subtype, or from IgA, IgE, IgD or IgM, including chimeric forms thereof, e.g., a chimeric IgG1/2 hinge region.

Since the disulfide bonds in different IgG forms of antibody are various, thus drug/antibody ratios (DARs) with the thiol-ether conjugation (such as through the Michael addition reaction of a maleimide of a drug/linker complex and a cysteine in an antibody) can be various. For instance, the DARs (or “n” in this application) can be up to 30 for IgG2, IgG3 or IgG4 form of an ADC.

The term “immune effector cell” or “effector cell” as used herein refers to a cell within the natural repertoire of cells in the human immune system which can be activated to affect the viability of a target cell. The viability of a target cell can include cell survival, proliferation, and/or ability to interact with other cells.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45:1628-50, 1999 and Fellouse, F. A., et al, J. Mol. Biol., 373 (4): 924-40, 2007).

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, In111, Re186, Re188, Sm153, Bi212, P32, Pb212, Zr89, F18, and radioactive isotopes of Lu, e.g. Lu177); chemotherapeutic agents or drugs (e.g., tubulysin, maytansin, auristatin, DNA minor groove binders (such as PBD dimers), duocarmycin, topoisomerase inhibitor I or II (such as camptothecins or etoposides), RNA polymerase inhibitors, DNA alkylators, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed throughout this patent application.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g. polyethylenoxy, PEG, poly(2-methyl)ethylenoxy, polymethyleneoxy, polypropyleneoxy) and alkylamino (e.g. polyethyleneamino); and diacid ester and amides including succinate, succinamide, diglycolate, malonate, and caproamide. In various embodiments, linkers can comprise one or more amino acid residues, such as valine, phenylalanine, lysine, and homolysine.

The words “comprise”, “comprising”, “include”, “including” and “includes” when used in this specification and claims are intended to specify the presence of stated features, integers, components, or steps, but they do not preclude the presence or addition of one or more other features, integers, components, steps, or groups thereof. The novel conjugates disclosed herein are the antibody conjugates targeting tumor specific antigens. Examples of the conjugates and their synthesis are shown in the examples 1-482 below of the specification.

### the Antibody Drug Conjugate of the Invention.

The invention provides an antibody-drug conjugate that have enhancement of killing of tumor cells. The antibody-drug conjugate (ADC) contains a branch (side chain) linker wherein a group of an affinity small molecule or/and an affinity peptide (such as neurotensin peptide), or a small molecule chemotherapeutic drug at the terminal of the side chain linker that have synergies of cytotoxicities, resulting in enhanced treatment of the tumors and refractory diseases. The formulas of the ADC of the present invention are represented as:

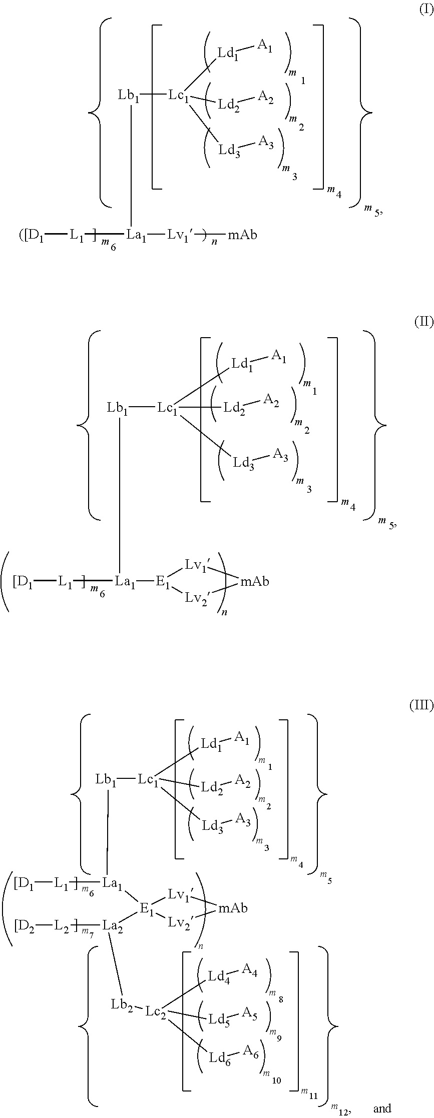

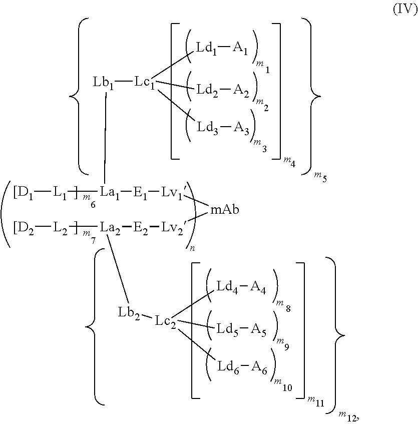

Wherein,
D₁ and D₂ are a cytotoxic agent; mAb is an antibody or antibody like protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker, which are independently selected from O, C, CH, CH₂, NH, S, N, NH—NH, N—N, N(R₃), N(R₃)N(R_{3′}), C(═O), C(═O)N, C(═O)NH, C(═O)N—N, C₂-C₈ of alkyl(alkylene), heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or 1˜8 natural or unnatural amino acids described in the definition; or polyethyleneoxy unit of formula (OCH₂CH₂) POR₃, (OCH₂CH₂)ₚ, OCH₂CH₂)ₚNHR₃, (OCH₂CH(CH₃)) POR₃, NH(CH₂CH₂O)ₚR₃, NH(CH₂CH(CH₃)O)ₚR₃, N[(CH₂CH₂O)ₚR₃][(CH₂CH₂O)ₚR_{3′}], (OCH₂CH₂)ₚCOOR₃, CH₂CH₂ (OCH₂CH₂)ₚCOR₃, or CH₂CH₂ (OCH₂CH₂)ₚCOOR₃, wherein p and p′ are independently an integer selected from 0 to about 1000, or combination thereof; wherein R₃ and R_{3′} are independently H, OH, C(═O) H, C(═O) CH₃, NH, C(═O), C(═O)NH, C₄˜C₆ glycose, C₁-C₈ of alkyl, absent; or combination above thereof;
In further embodiments, L₁, L₂, La₁, La₂, Lb₁, Lb₂, Lc₁, Lc₂, Ld₁, Ld₂, Ld₃, Ld₄, Ld₅, and Ld₆ are 1˜4 natural or unnatural amino acids, C₁-C₈ of gamma amino-alkyl, aminoalkylcarboxyl, thioalkylcarboxyl or oxylalkylcarboxyl, or C₃-C₁₈ of gamma amino-benzyl or aromatic of ester, ether, urea, carbamate, carbonate, thiourea, thioether, thiourea, or amide, wherein aromatic is selected from phenyl, phenol, benzyloxyl, benzyloxyl, benzylamino, phenylcarboxyl, oxyl-phenyl-carboxyl, amino-phenyl-carboxyl, benzyloxycarbonyl, benzylaminocarbonyl, triazol, tetrazol, pyridinyl, pyrimidinyl, pyrazineyl, oxazole, thiazole. In addition, lysine and or glutamic acid among natural or unnatural amino acids can have a side chain of a polyethyleneoxy unit of formula C(O)(CH₂CH₂O)ₚR₃, or NH(CH₂CH₂O)ₚR₃ linked to the amino or carboxy group of lysine and or glutamic acid respectively, wherein p and R₃ are described above; And one or several of L₁, L₂, La₁, La₂, Lb₁, Lb₂, Lc₁, Lc₂, Ld₁, Ld₂, Ld₃, Ld₄, Ld₅, and Ld₆ can be absent thereof.

In addition, La₁ and La₂ are independently a peptide having 1˜8 amino acids, which contain one or several lysine, glutamic acid, aspartic acid, cysteine, glutamine, asparagine and tyrosine, and these amino acids that link A₁, A₂, A₃, A₄, A₅ and A₆ through Lb₁, Lb₂, Lc₁, Lc₂, Ld₁, Ld₂, Ld₃, Ld₄, Ld₅, and Ld₆ accordingly;

E₁ and E₂ are a joint group selected from CH, CH₂, CH—CH, NH, NHNH, N(R₃), N(R₃)N(R_{3′}), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1˜4 units of aminoacids, preferably selected from aspartic acid, glutamic acid, arginine, histidine, lysine, serine, threonine, asparagine, glutamine, cysteine, selenocysteine, tyrosine, phenylalanine, glycine, proline, tryptophan, alanine; or one of the following structures:

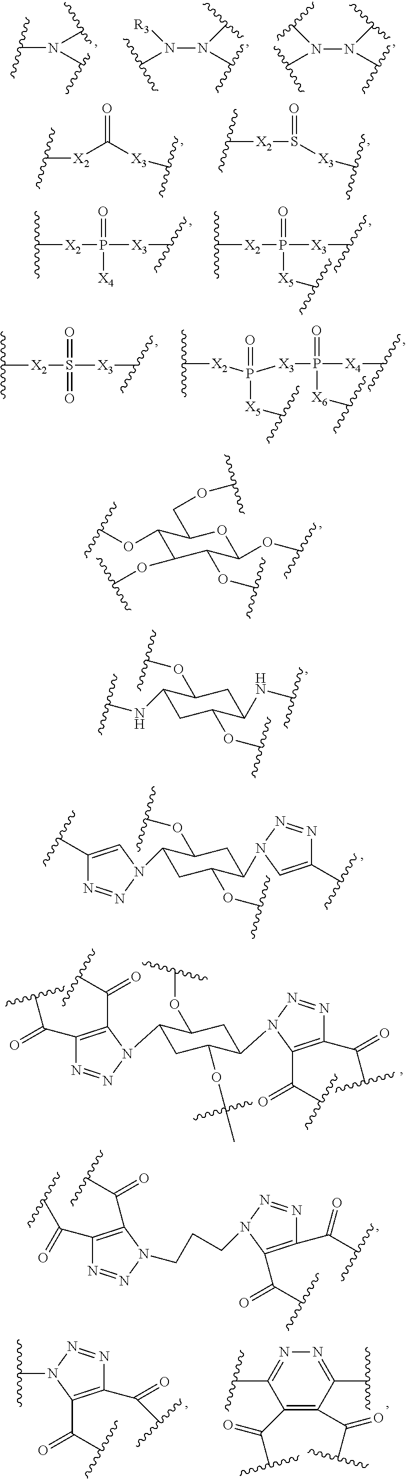

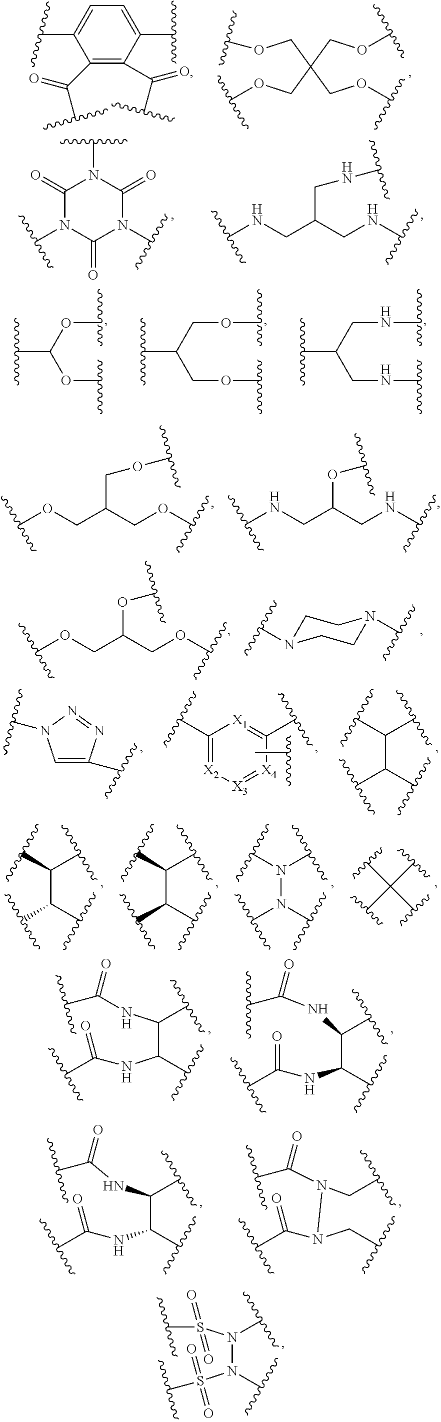

wherein [symbol] is the site of linkage; X₁, X₂, X₃, X₄, X₅, or X₆, are independently selected from NH; NHNH; N(R₃); N(R₃)N(R_{3′}); O; S; C₁-C₆ of alkyl; R₃ and R_{3′} are H, C₁-C₆ of alkyl; In addition, E₁ can be absent, thus La₁ or/and La₂ can directly link to Lv₁′ or Lv₂′;
m₁, m₂, m₃, m₄, m₅, m₆, m₇, m₈, m₉, m₁₀, m₁₁ and m₁₂ are independently 1, 2, 3, 4, 5, 6, 7, 8, 9, 10, and these numbers may have a decimal; in addition, m₂, m₃, m₈, m₉ and/or m₁₀ can be 0, thus Ld₂-A₂, Ld₃-A₃, Ld₅-A₅, and/or Ld₆-A₆ can be absent;
wherein Lv₁′ and Lv₂′ are independently or jointly having the following structures:

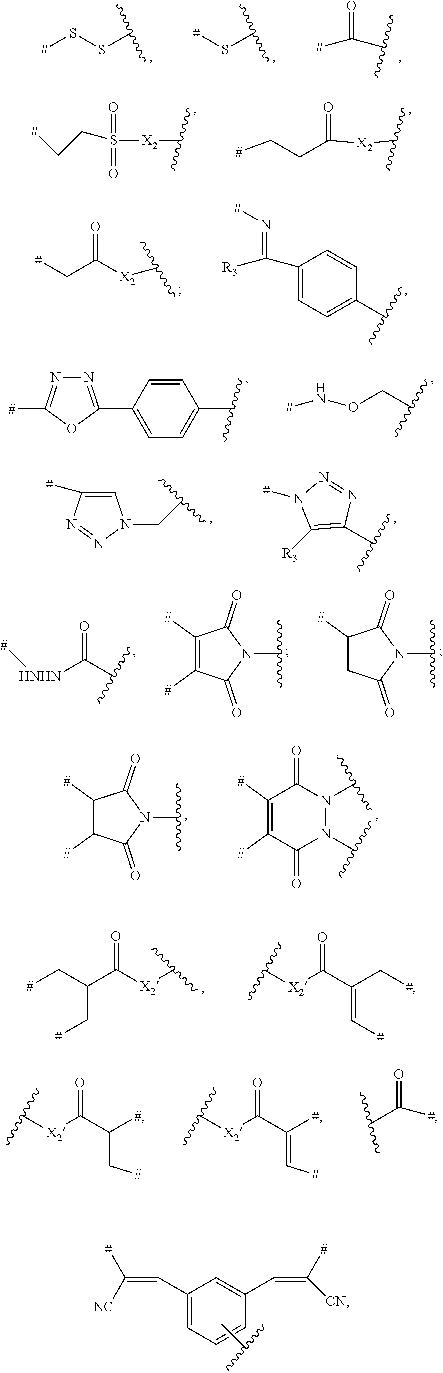

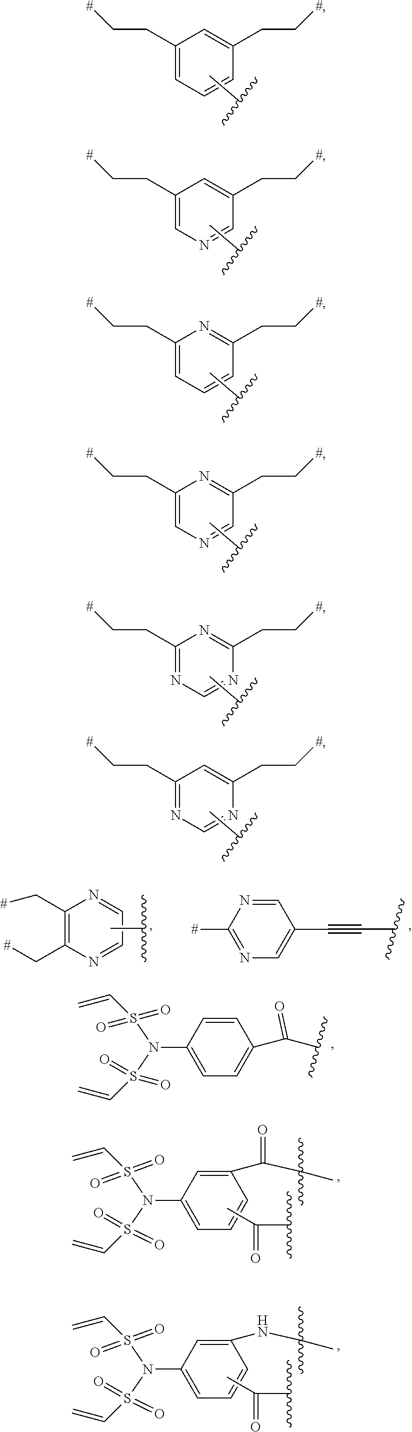

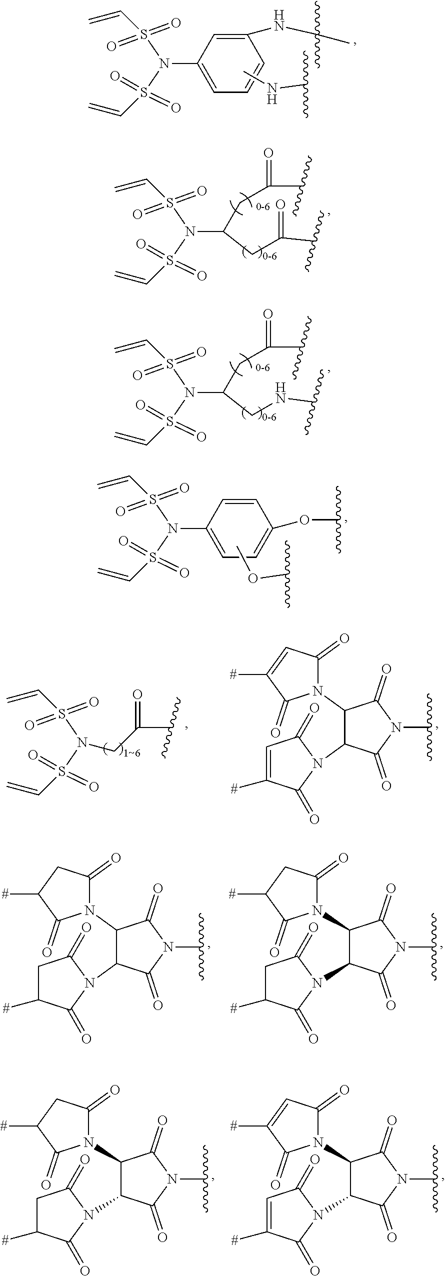

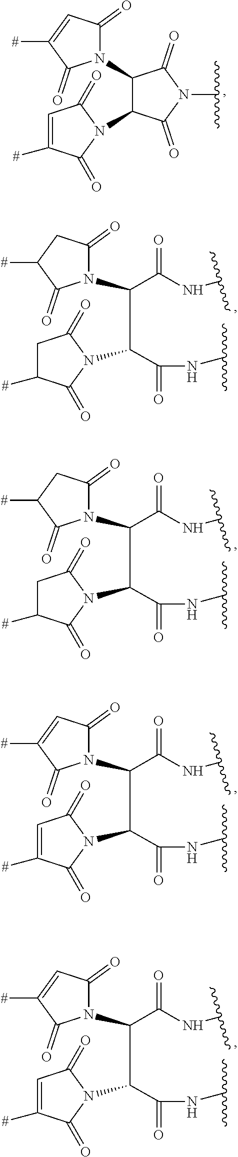

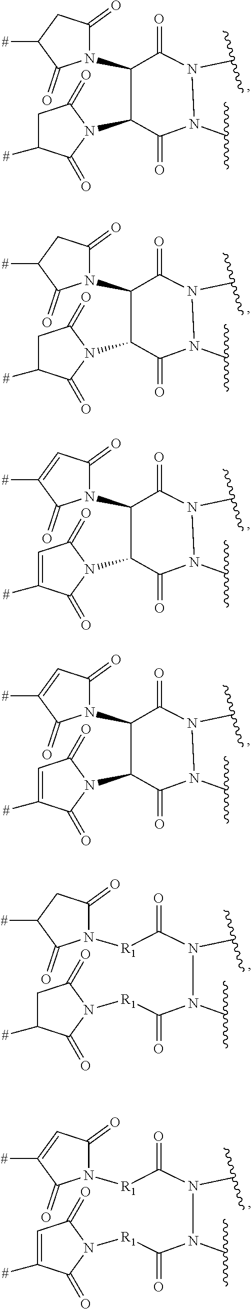

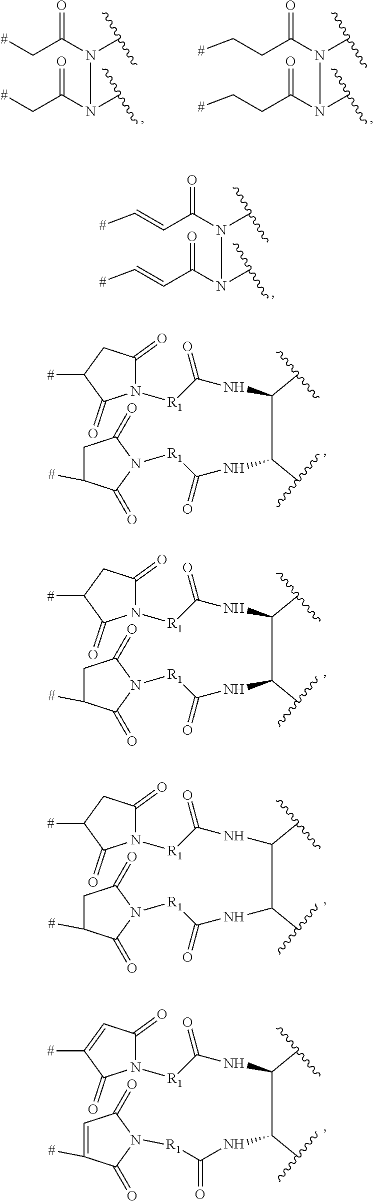

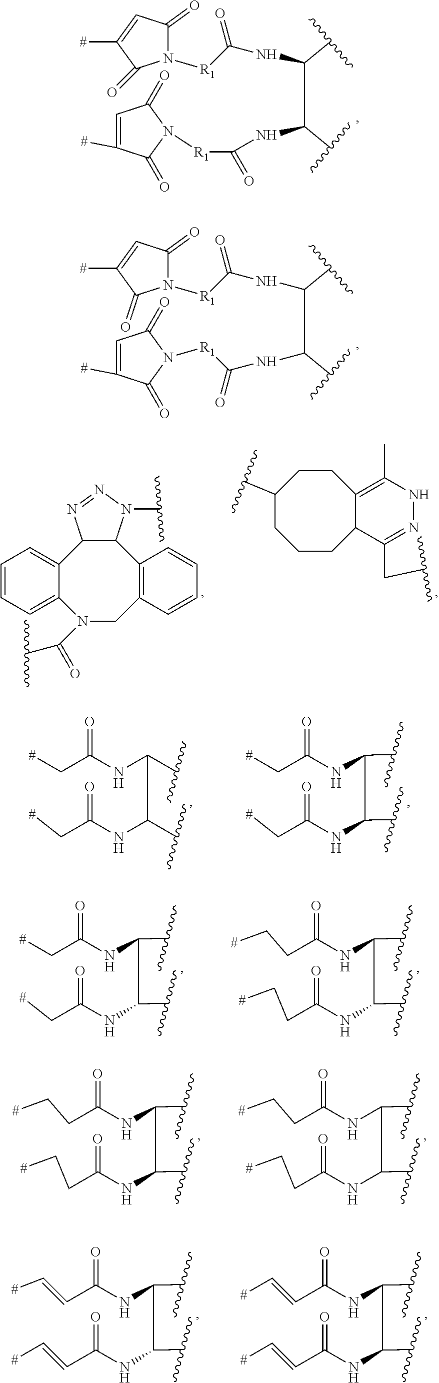

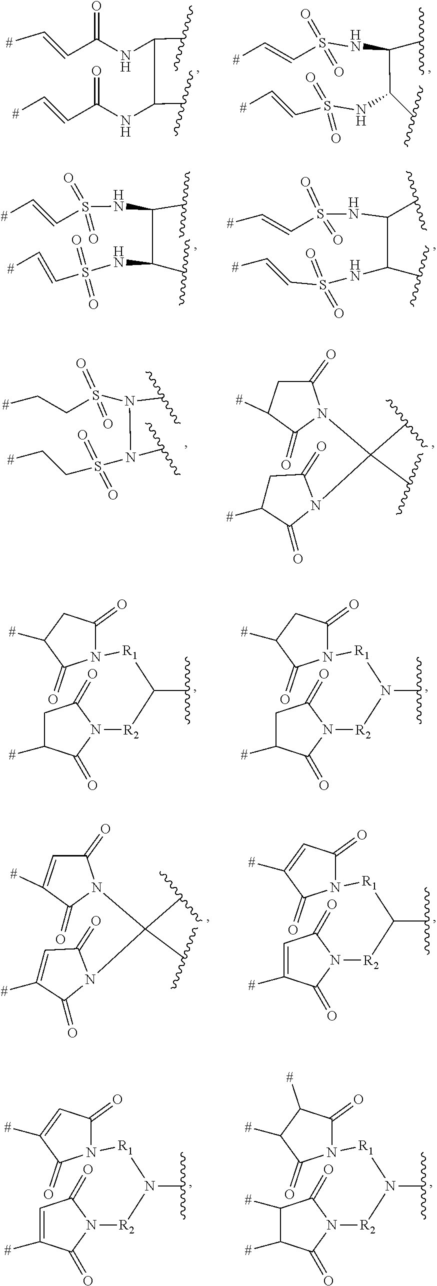

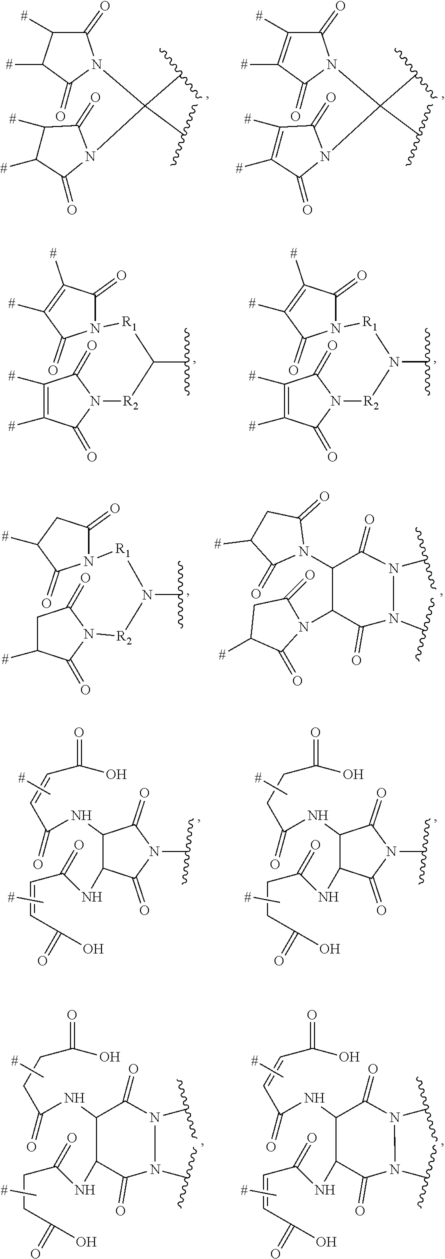

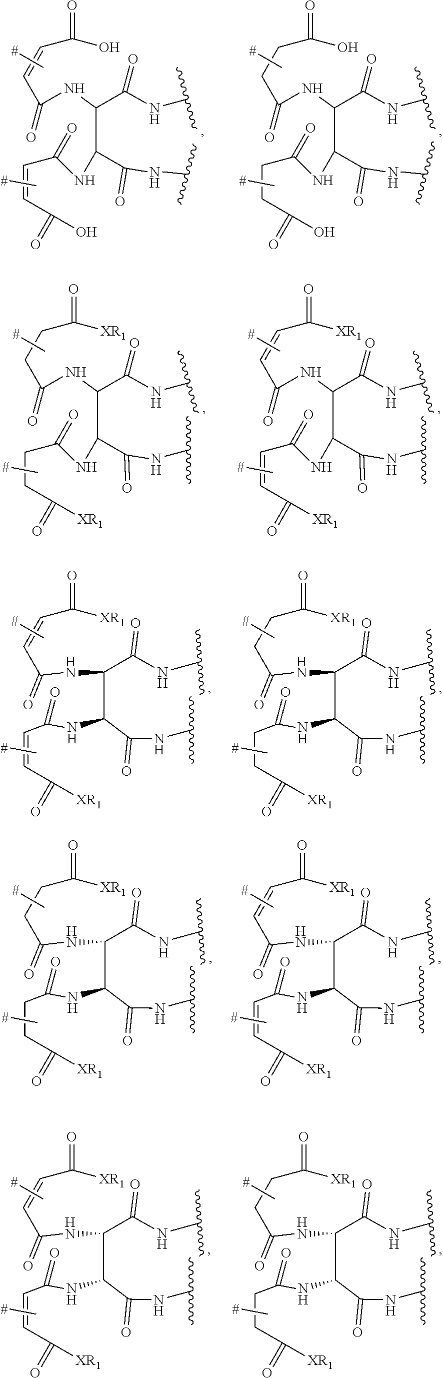

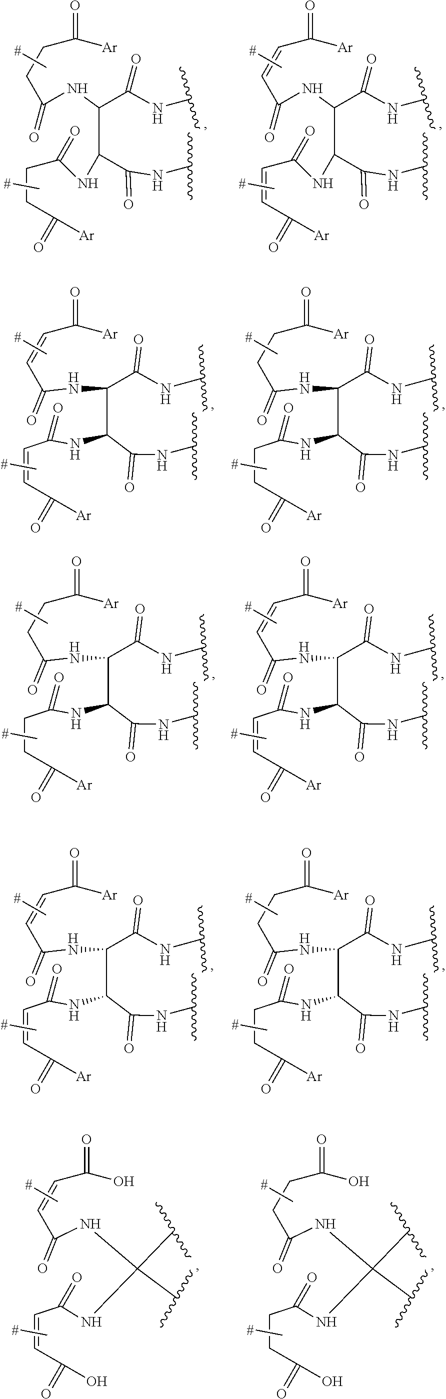

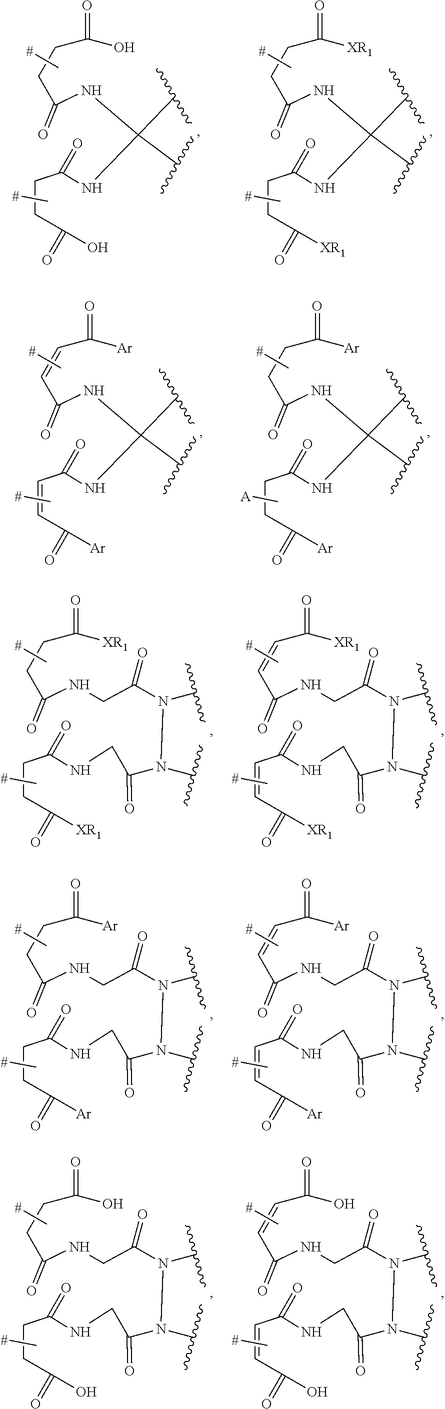

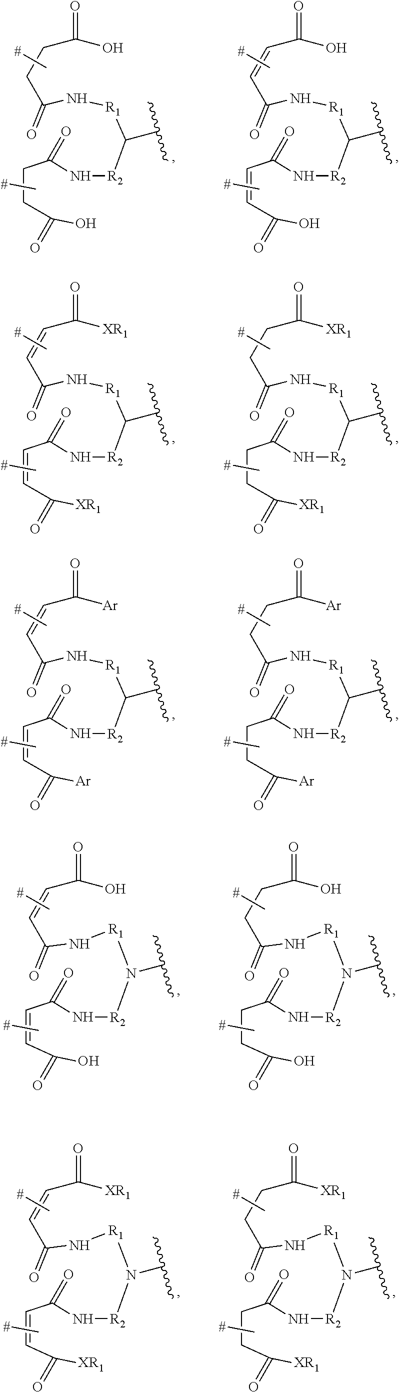

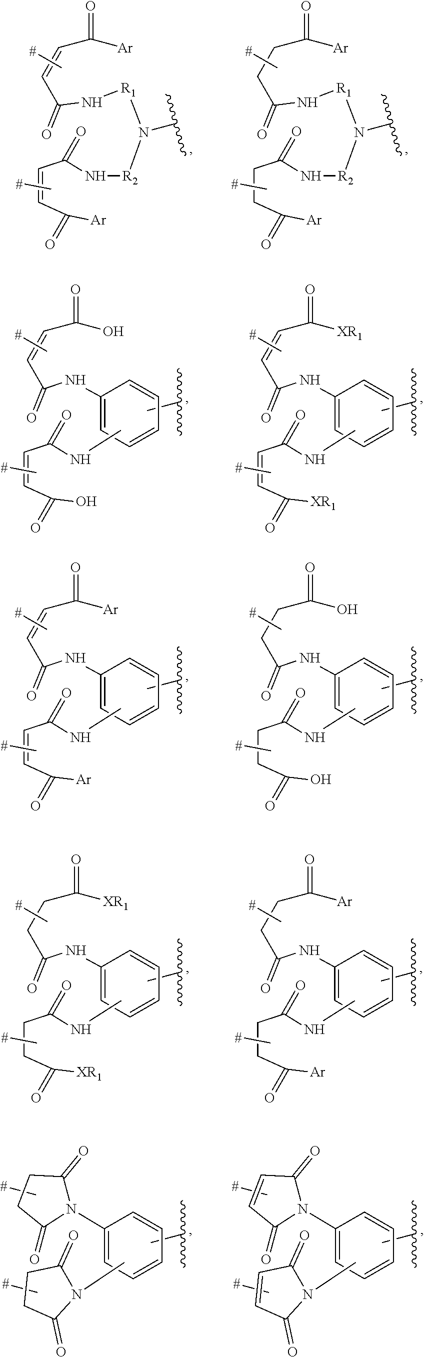

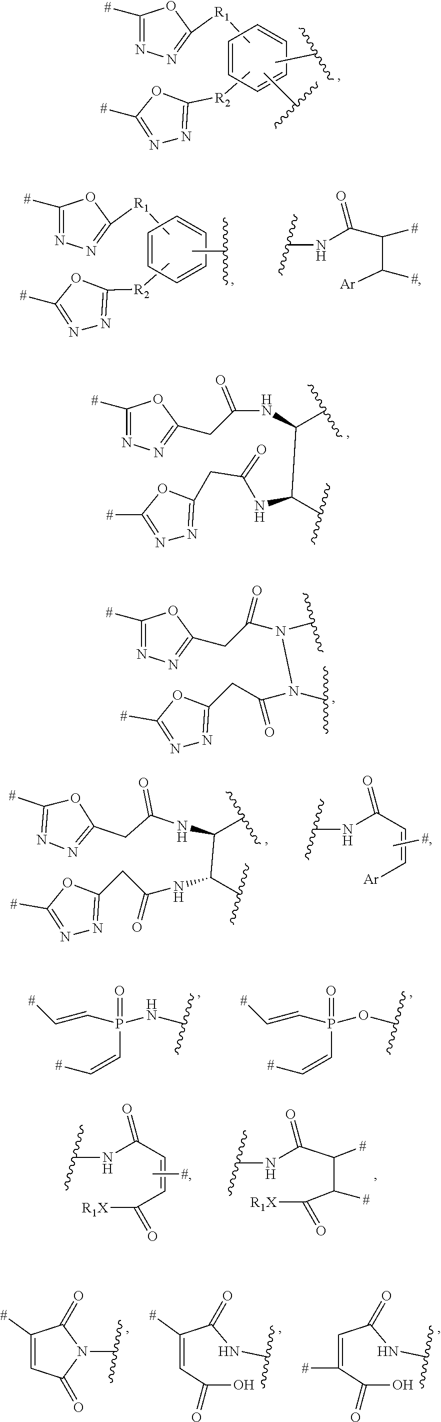

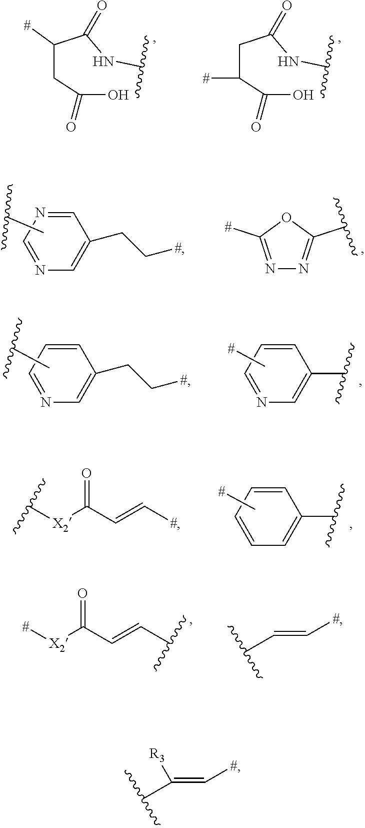

wherein [symbol] is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; wherein R₁ and R₂ are H, C₁-C₆ of alkyl or a peptide containing 1˜4 units of aminoacids; X₁′, X₂′ and X are O, NH, S, CH₂; the connecting bond “—” in the middle of the two atoms means it can link either one of the two atoms, Ar is an aromatic group; A₁, A₂, A₃, A₄, A₅ and A₆ are independently an affinity ligand including affinity peptide and cell-penetrating peptide (CPP), a ligand for bombesin receptors/neurotensin receptors (including neuropeptide-Y receptors), a chemotherapeutic drug, a stimulating agent, and/or a nucleoside antimetabolite/analogs, which have either the synergy with D₁ or D₂, or enhanced affinity for mAb.

In some embodiments, the affinity ligand/peptide including the cell-penetrating peptide (CPP) to a receptor on a faulty cell is EC50<100 nM. The CPP is a linear or cyclo peptide having less than 50 amino acids and containing one, two, or several arginines or lysines and enables to internalize (trafficking) over 40% of the ligand bound on a cell or help to internalize 40% of ADCs bound on a cell to cross the cell membrane in 2 hours.

In some embodiments, the affinity ligand/peptide including the cell-penetrating peptide (CPP) is independently selected from:

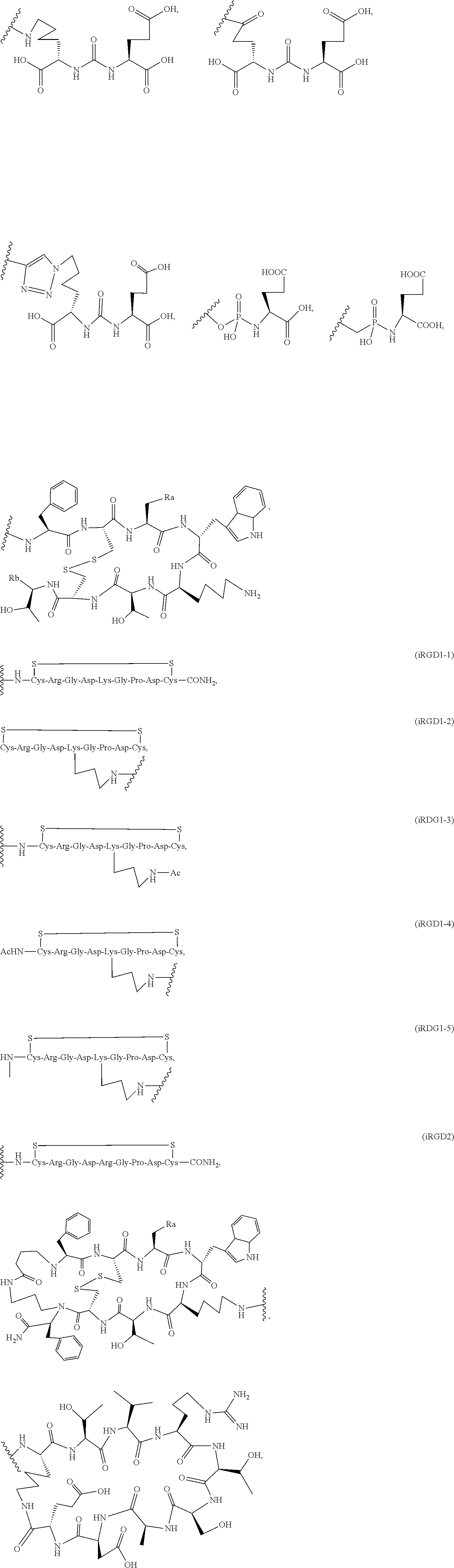

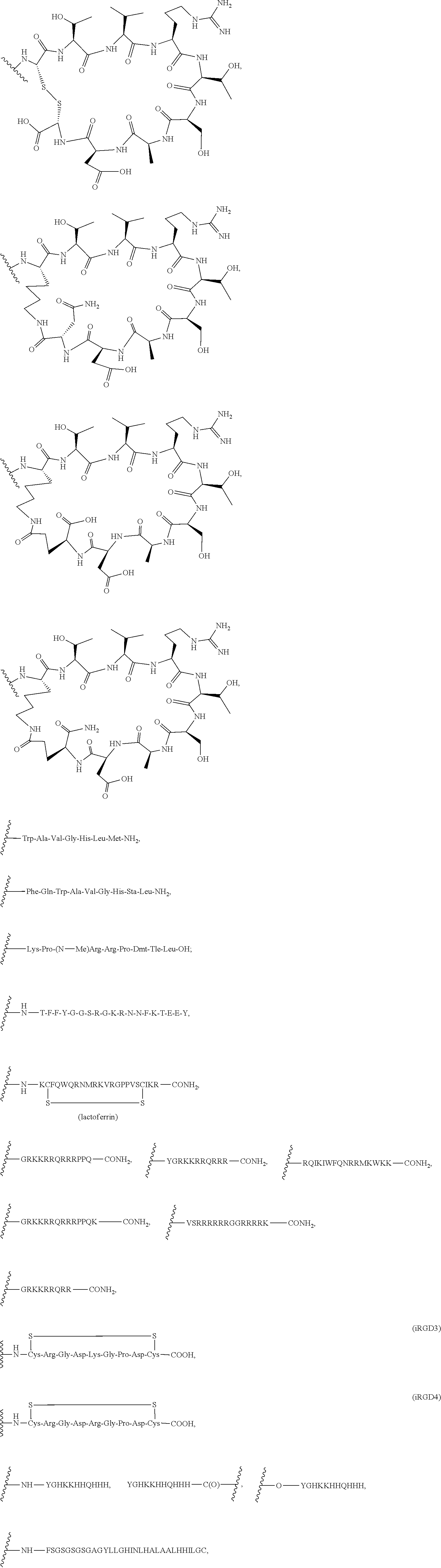

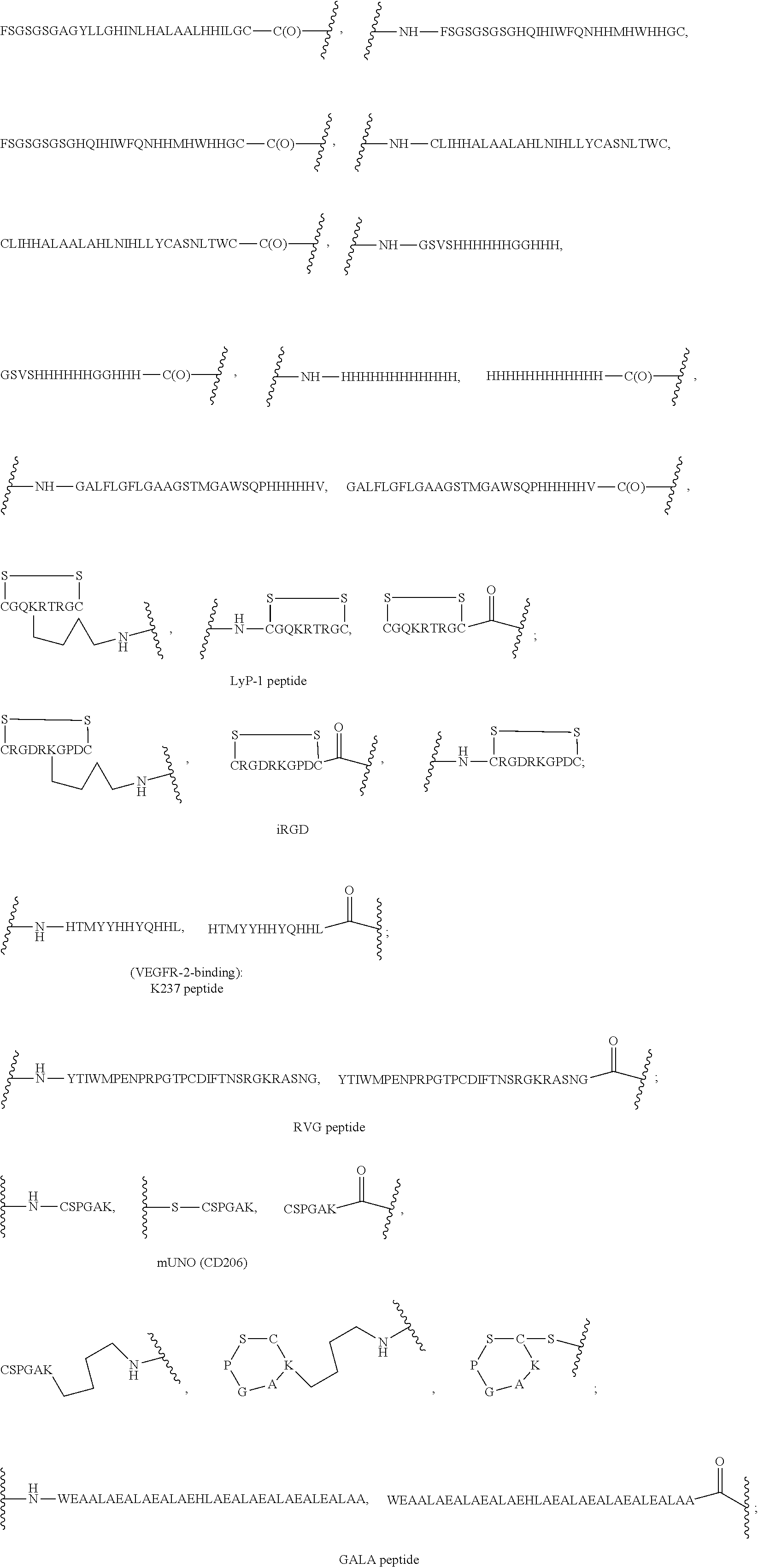

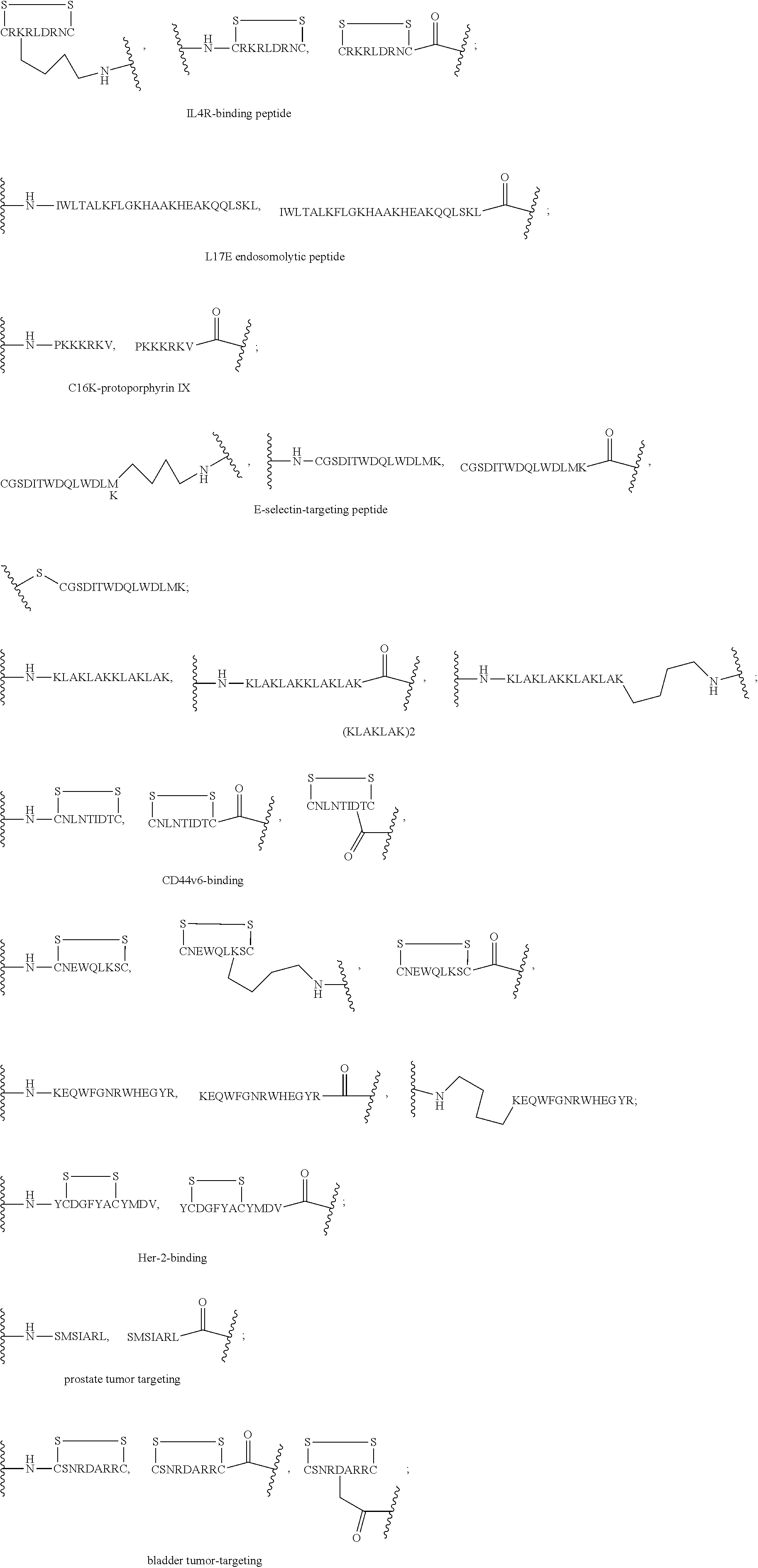

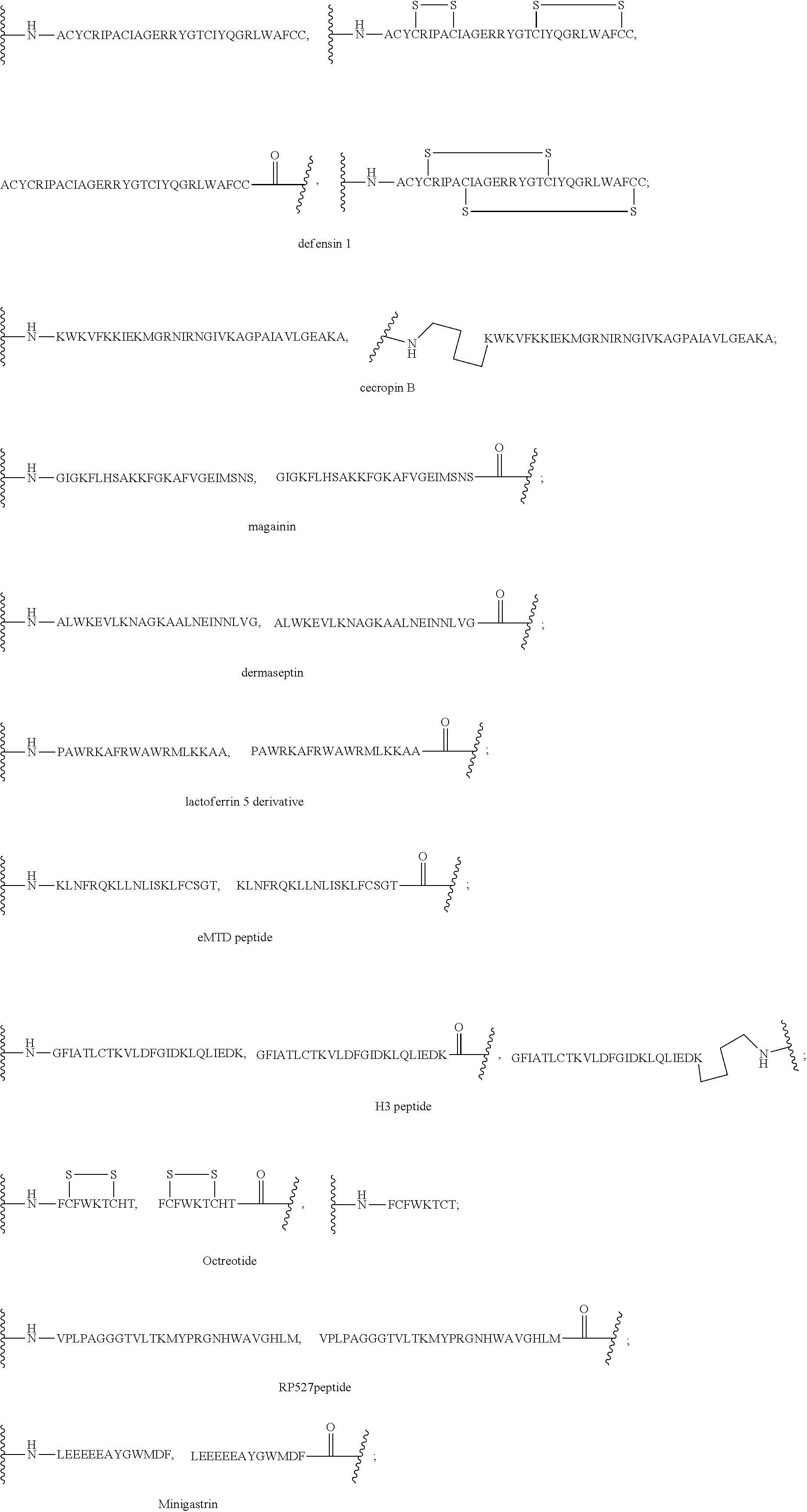

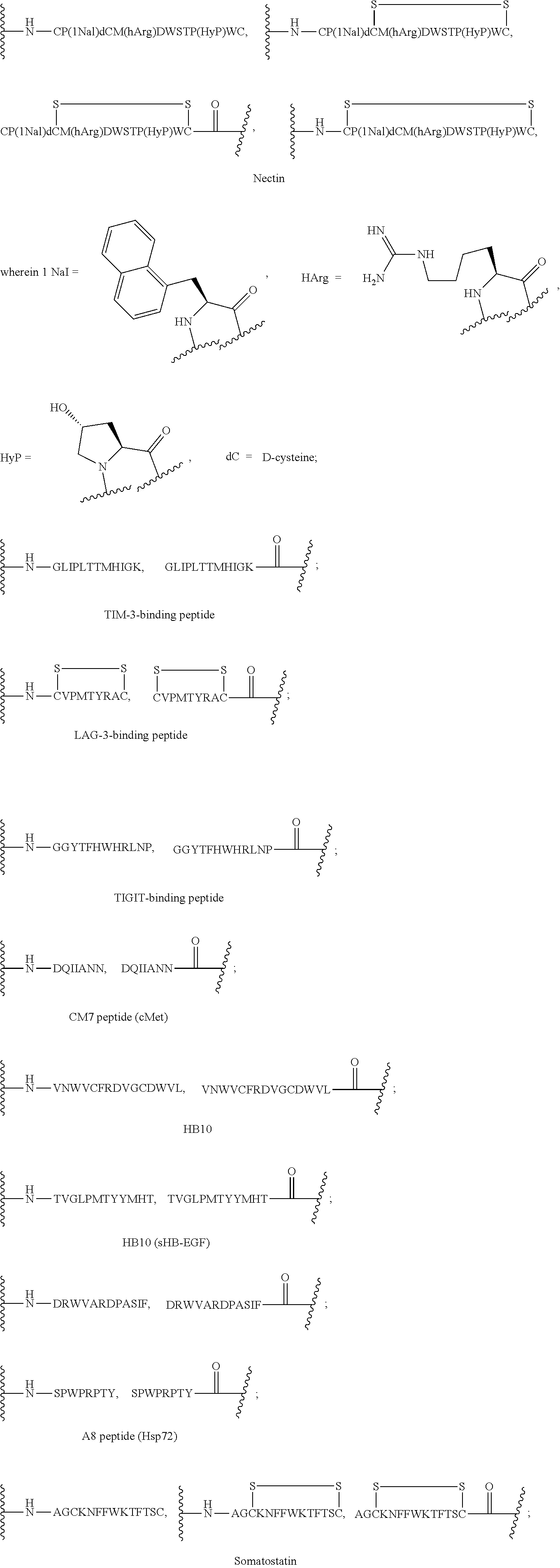

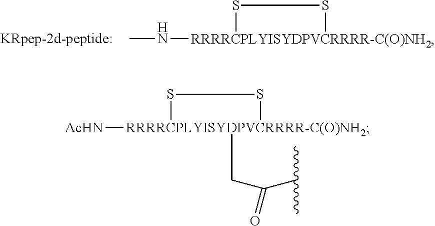

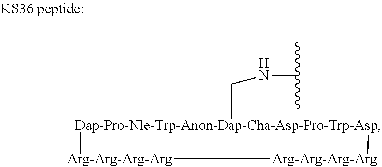

wherein Dap is(S)-2,3-Diaminopropanoic acid, Nle is L-Norleucine, Anon is(S)-2-Aminononanoic acid, Cha is L-Cyclohexylalanine. The others are natural amino acids.

KS58 peptide:

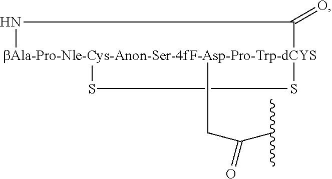

wherein βA1a is Beta-alanine, Nle is L-Norleucine, Anon is(S)-2-Aminononanoic acid, 4fF is 4-Fluoro-1-phenylalanine, dCys is D-cysteine;

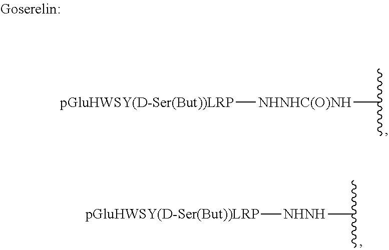

and its analogs as the structures shown below:

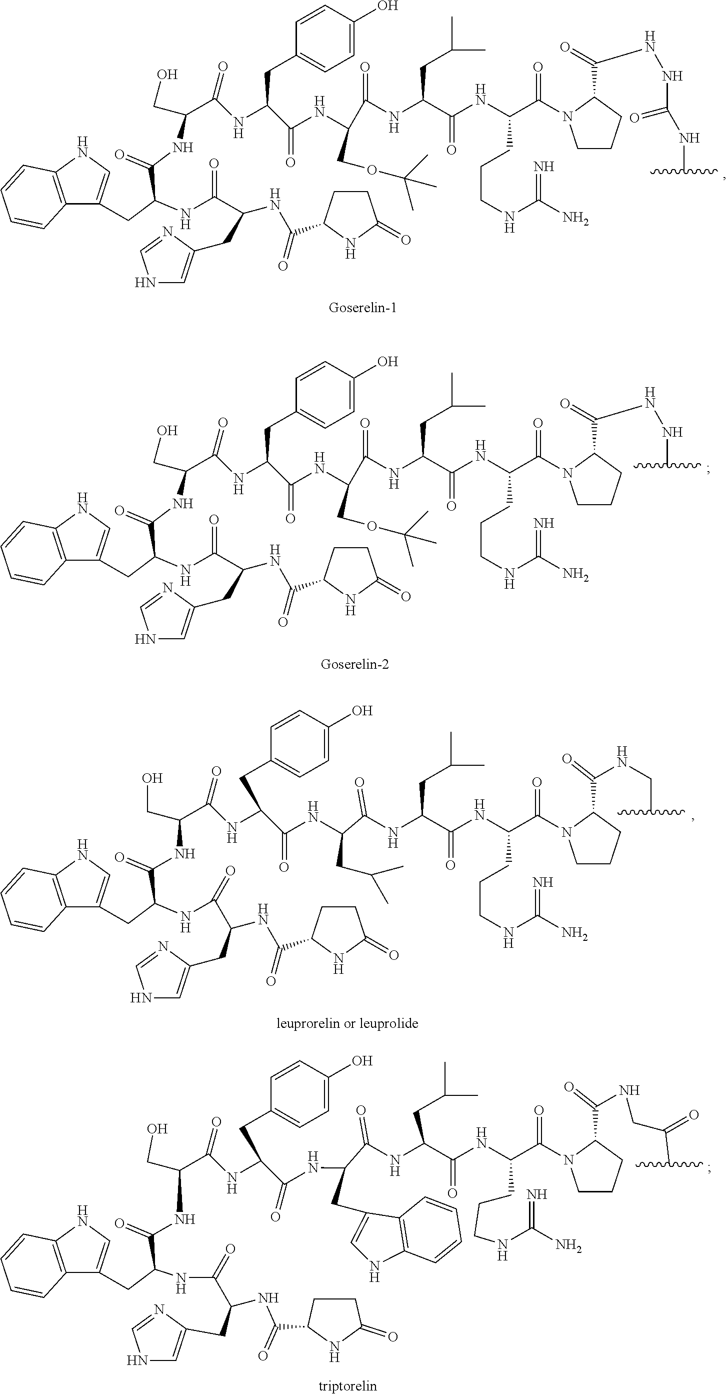

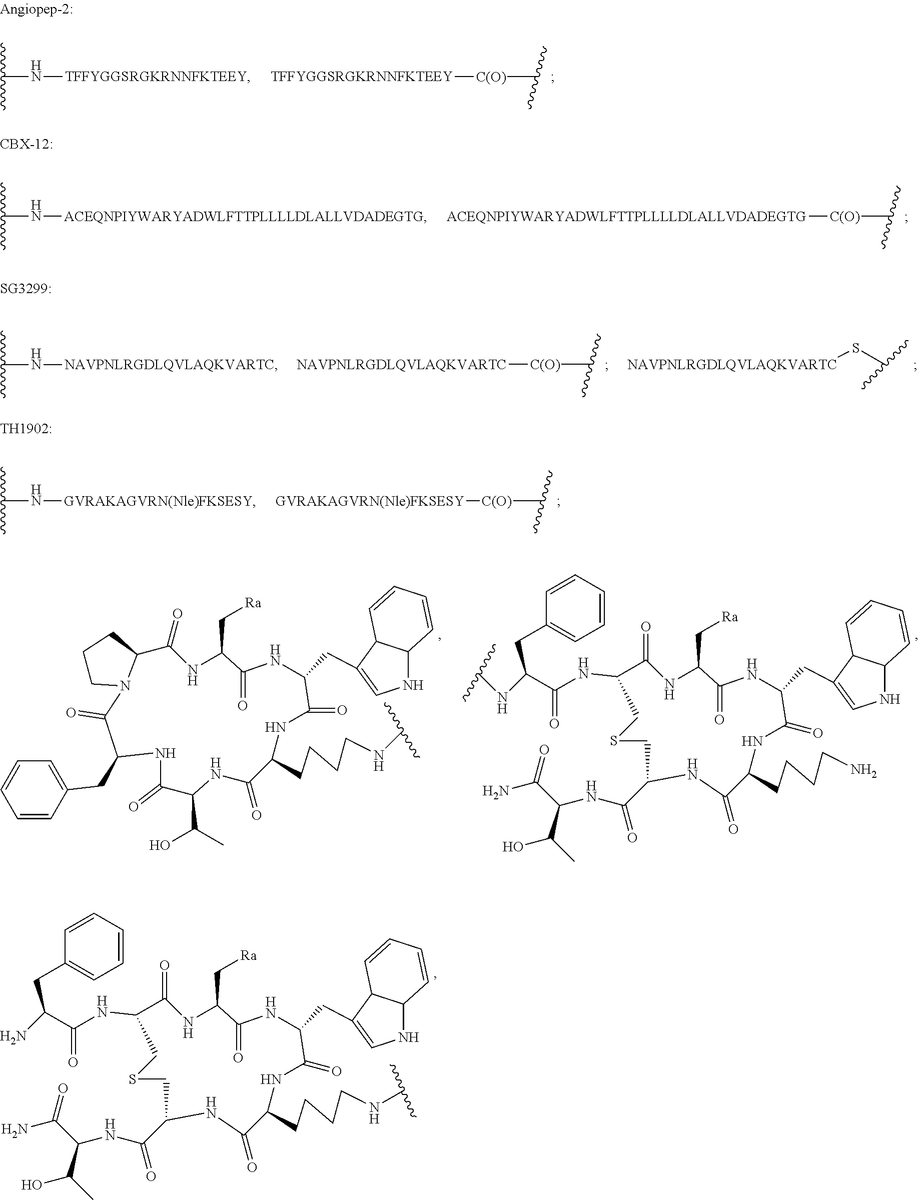

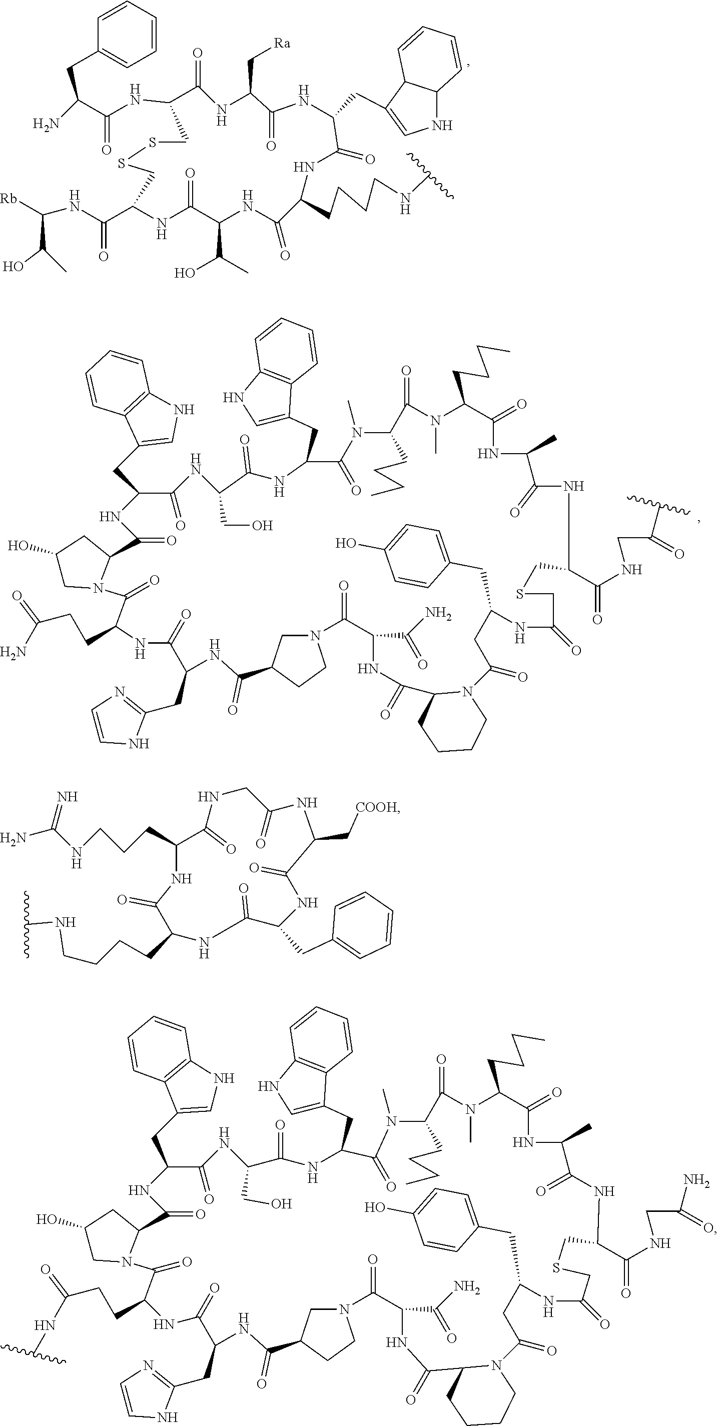

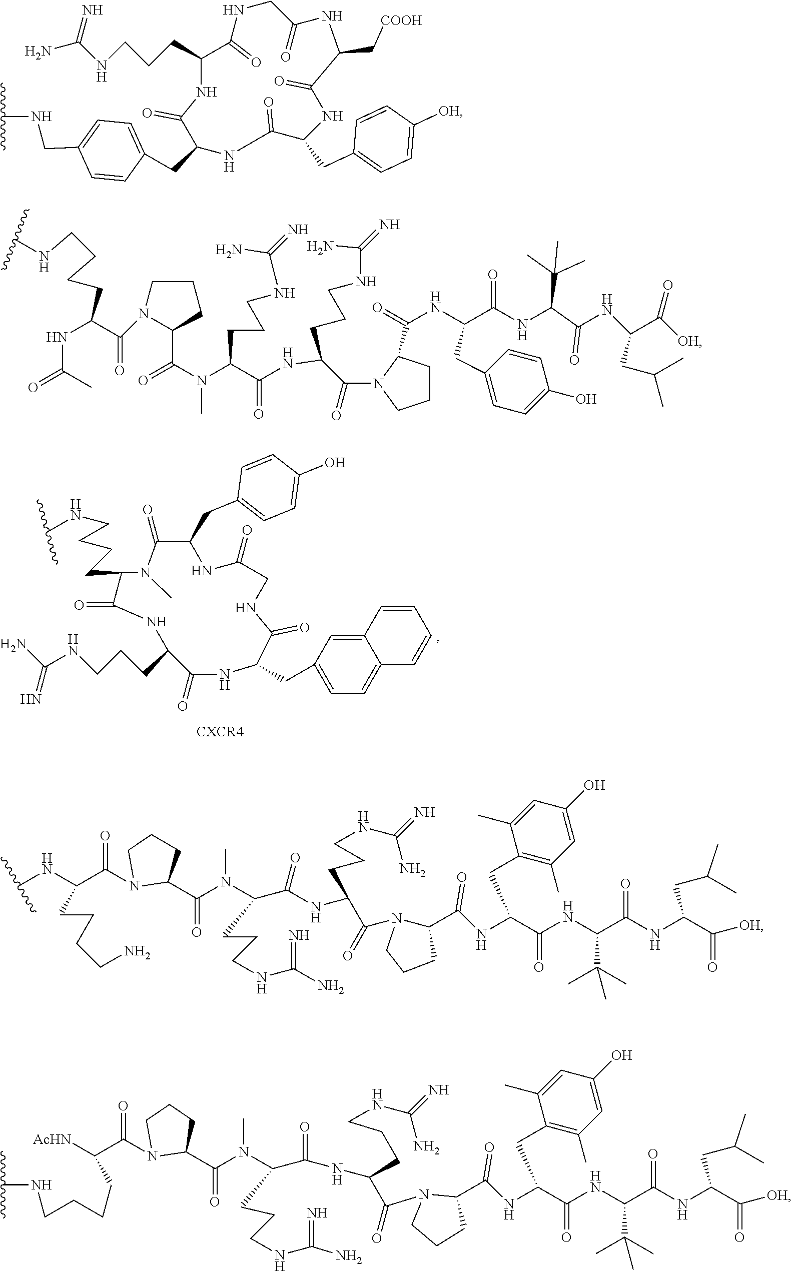

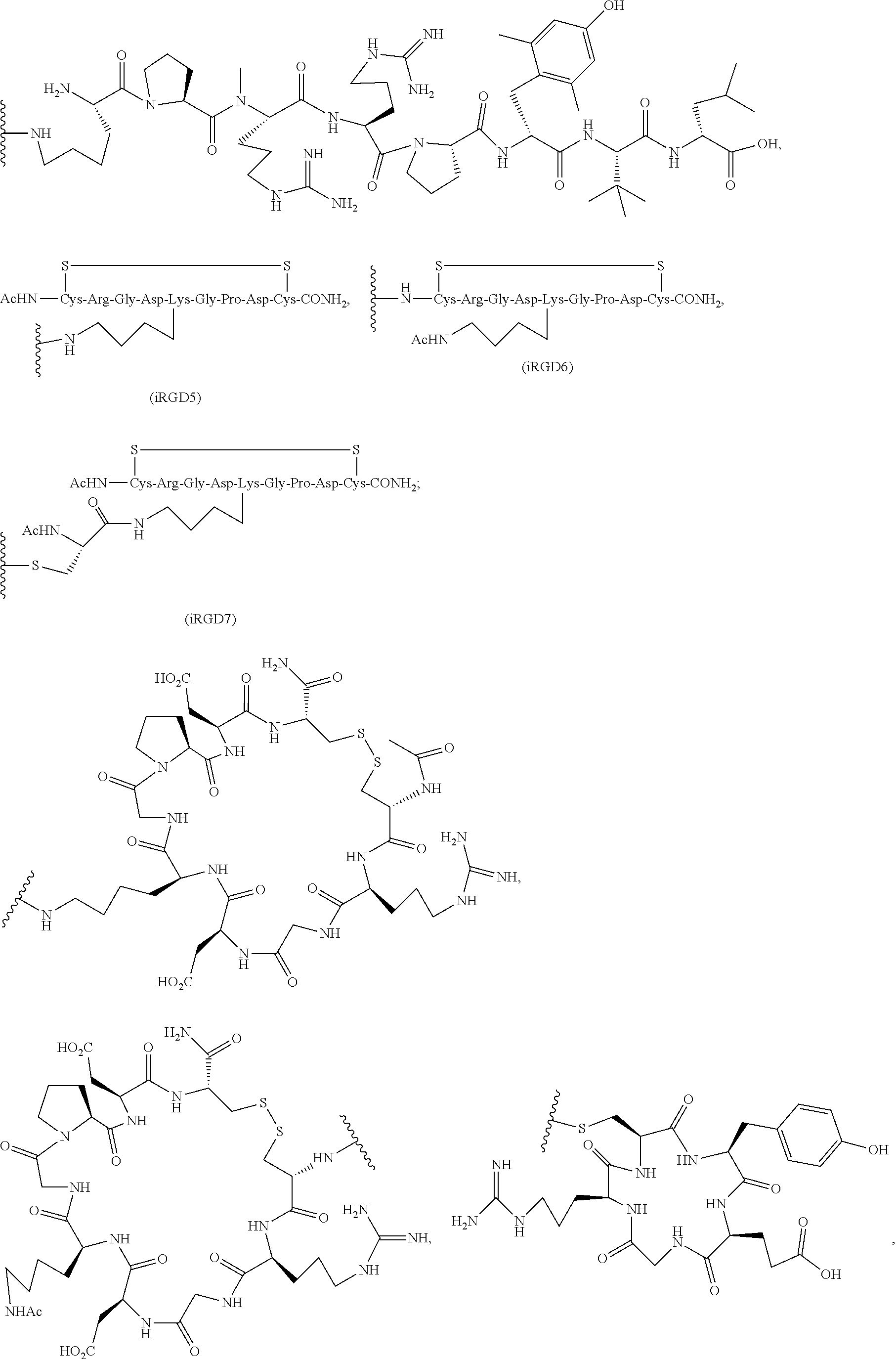

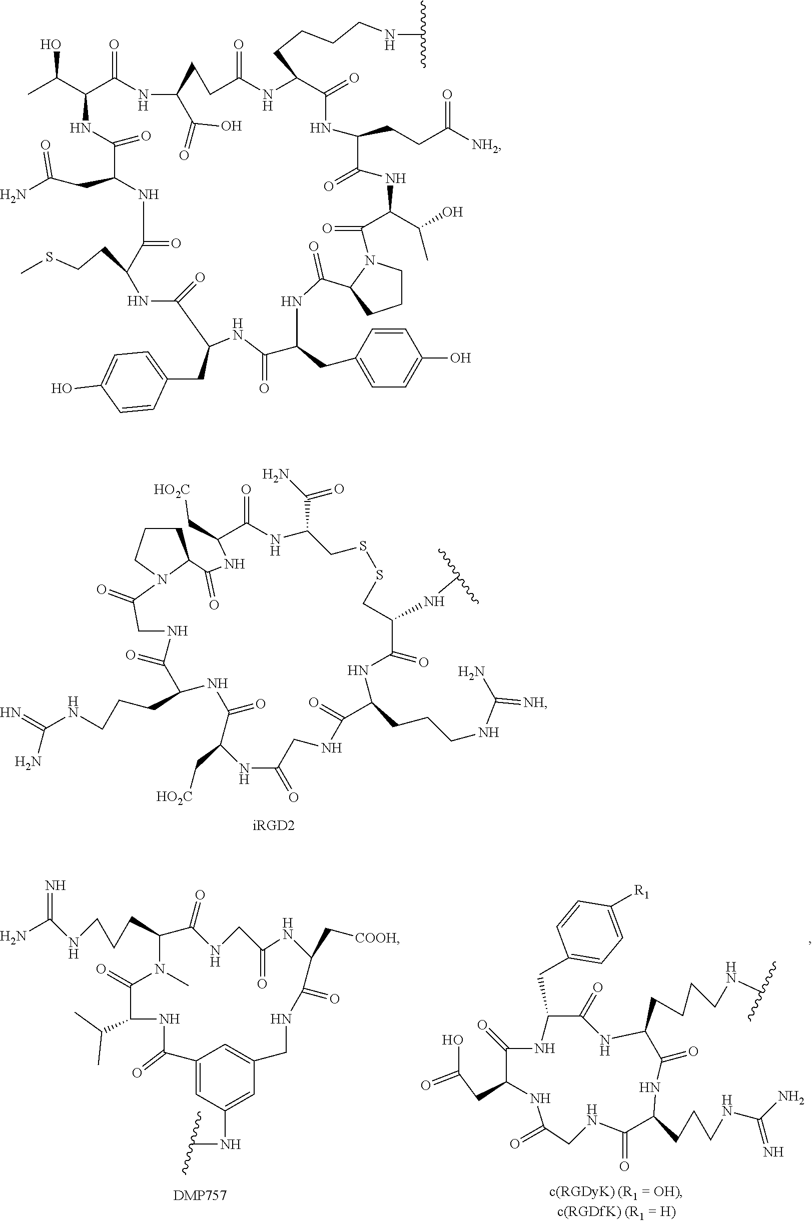

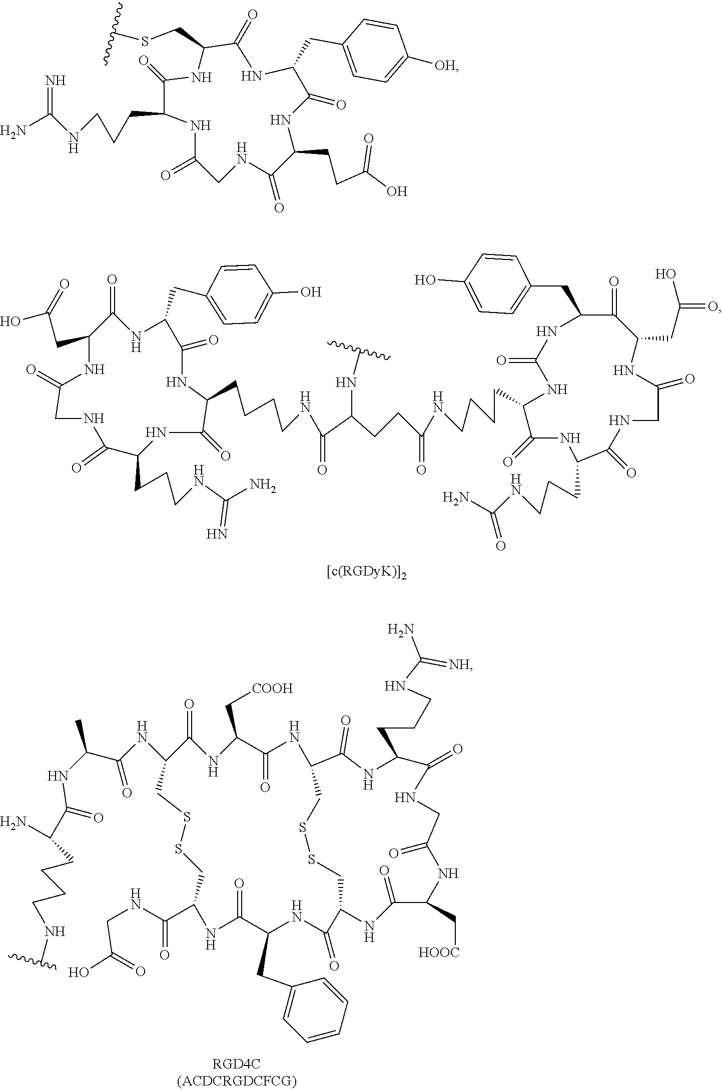

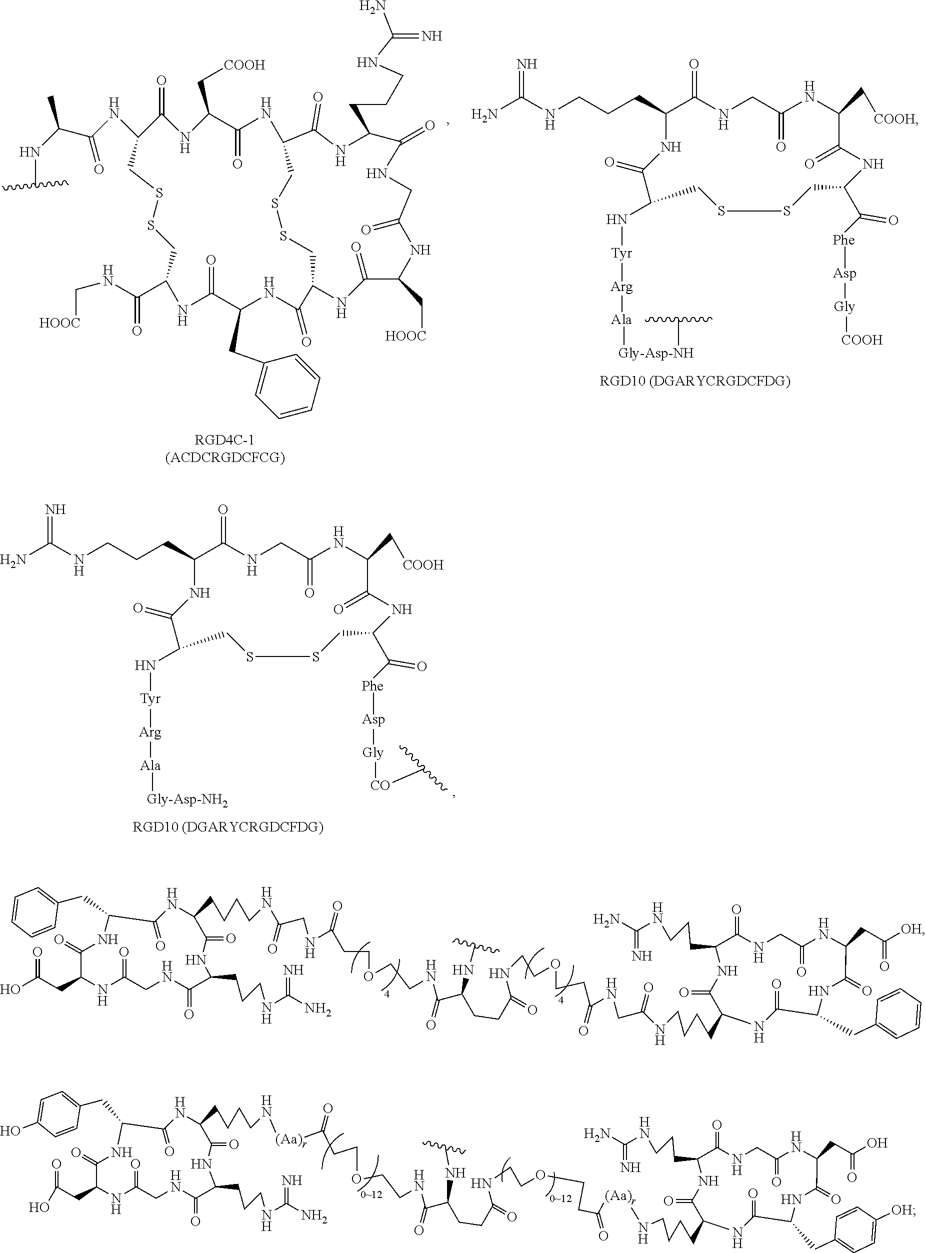

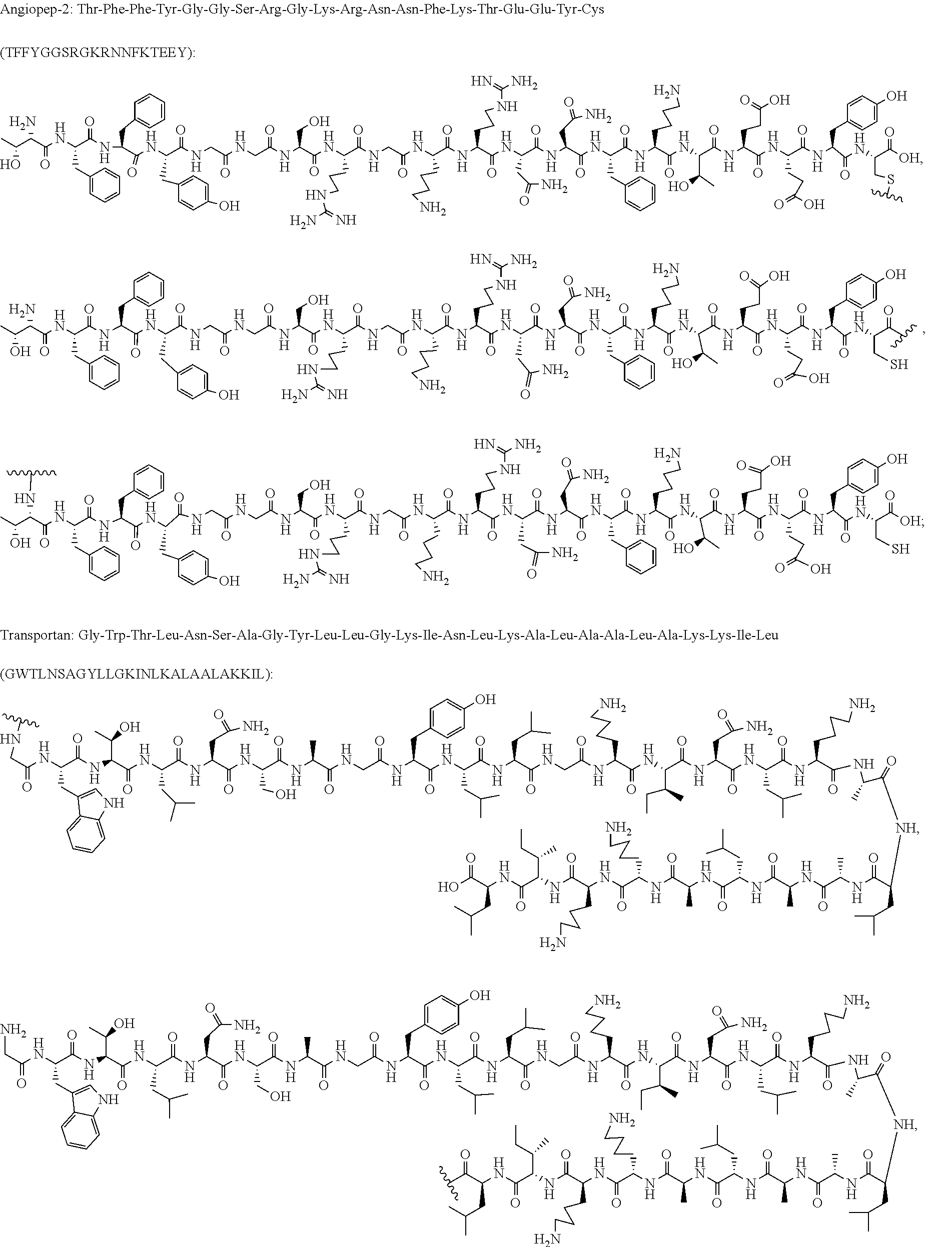

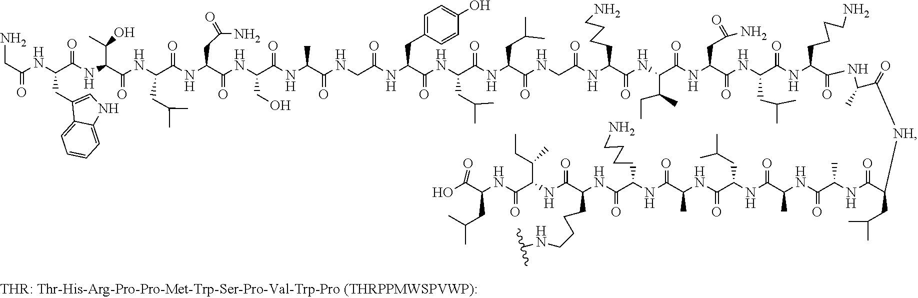

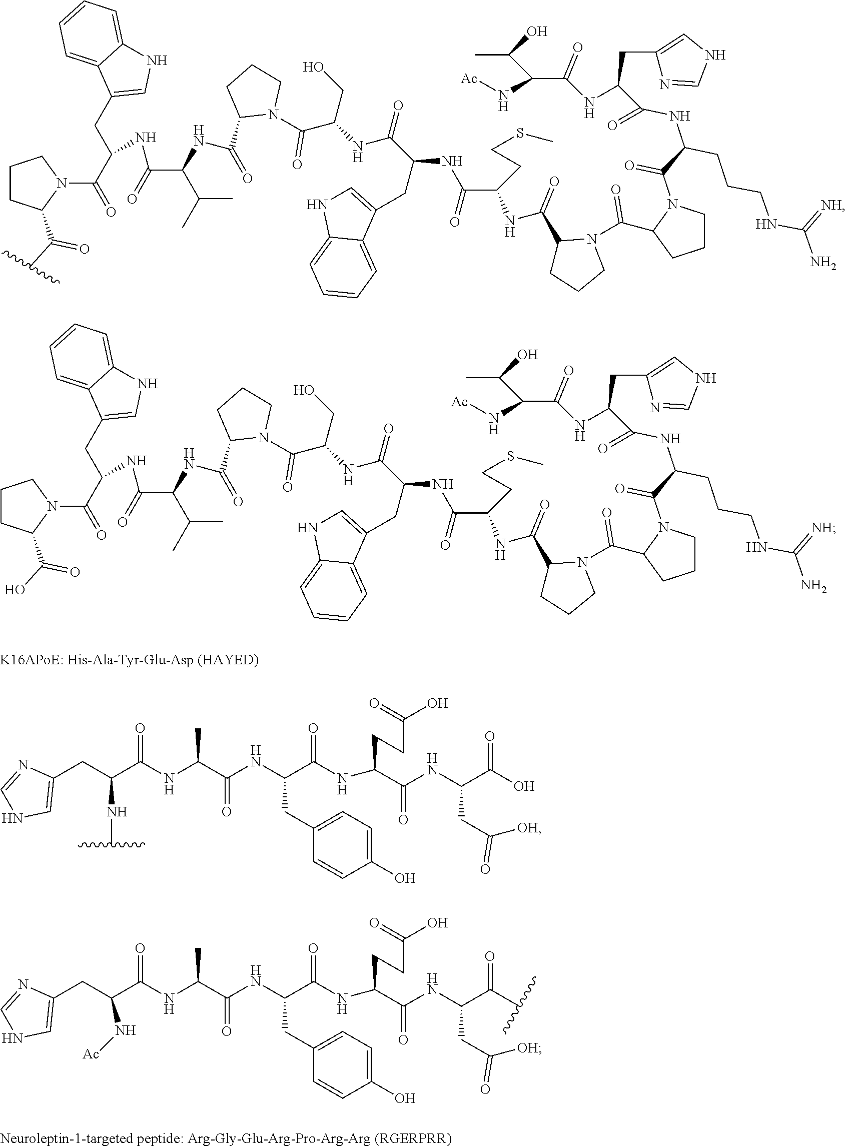

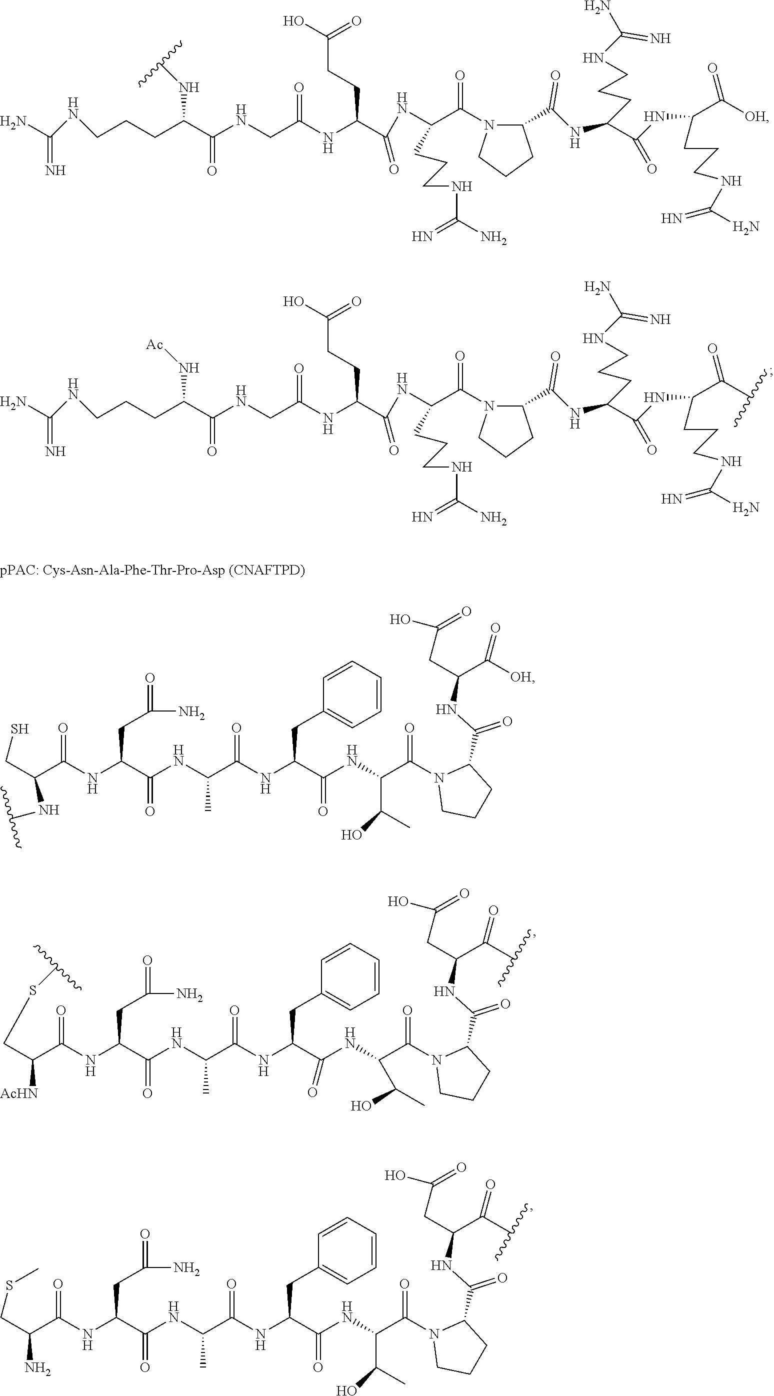

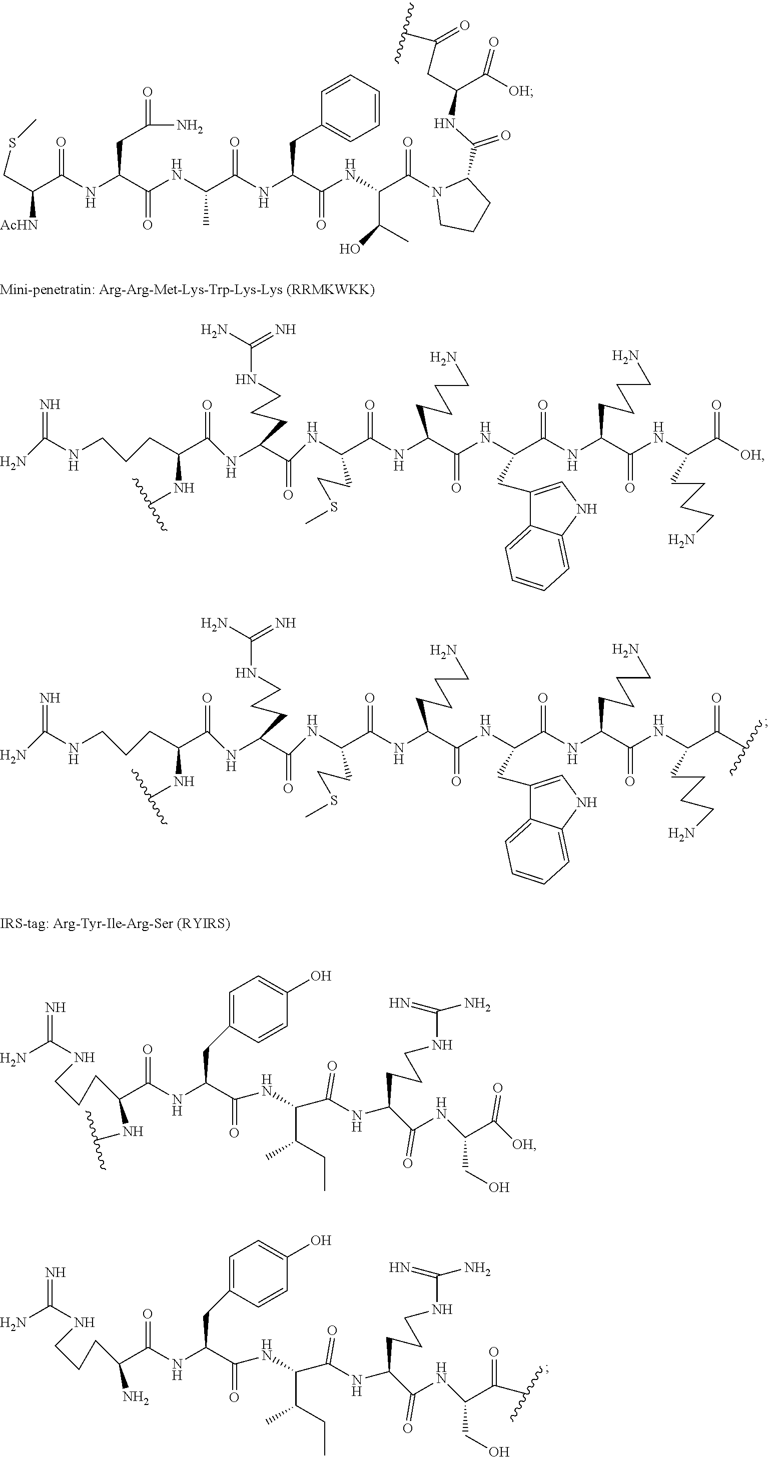

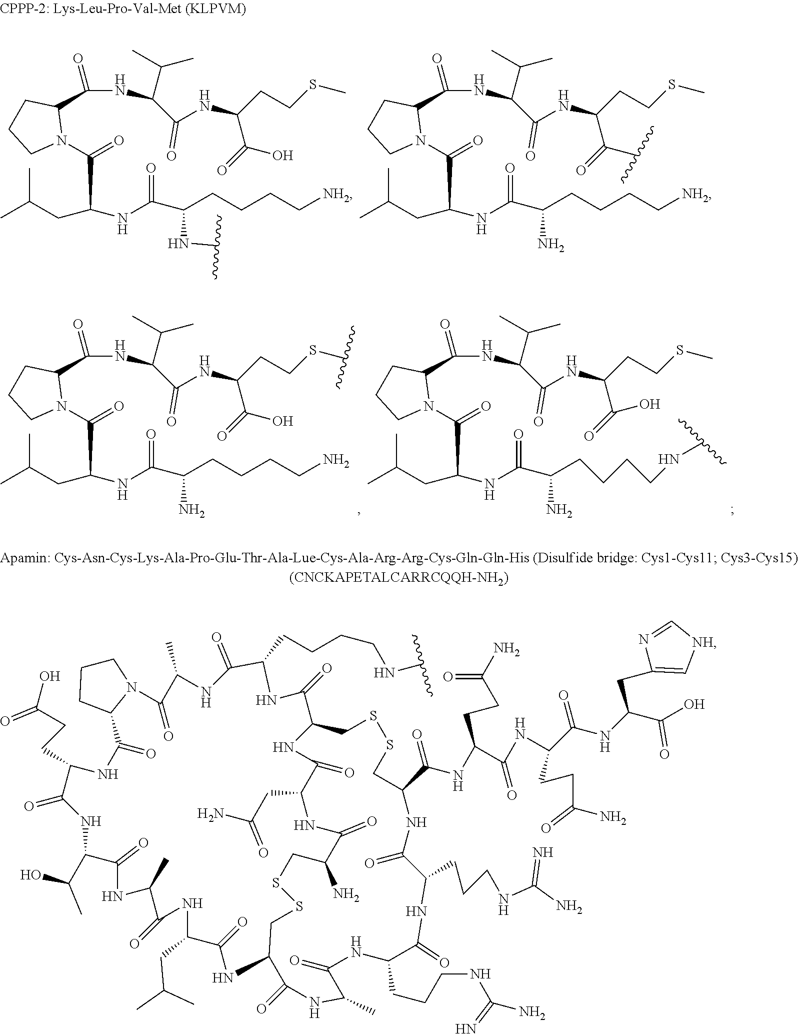

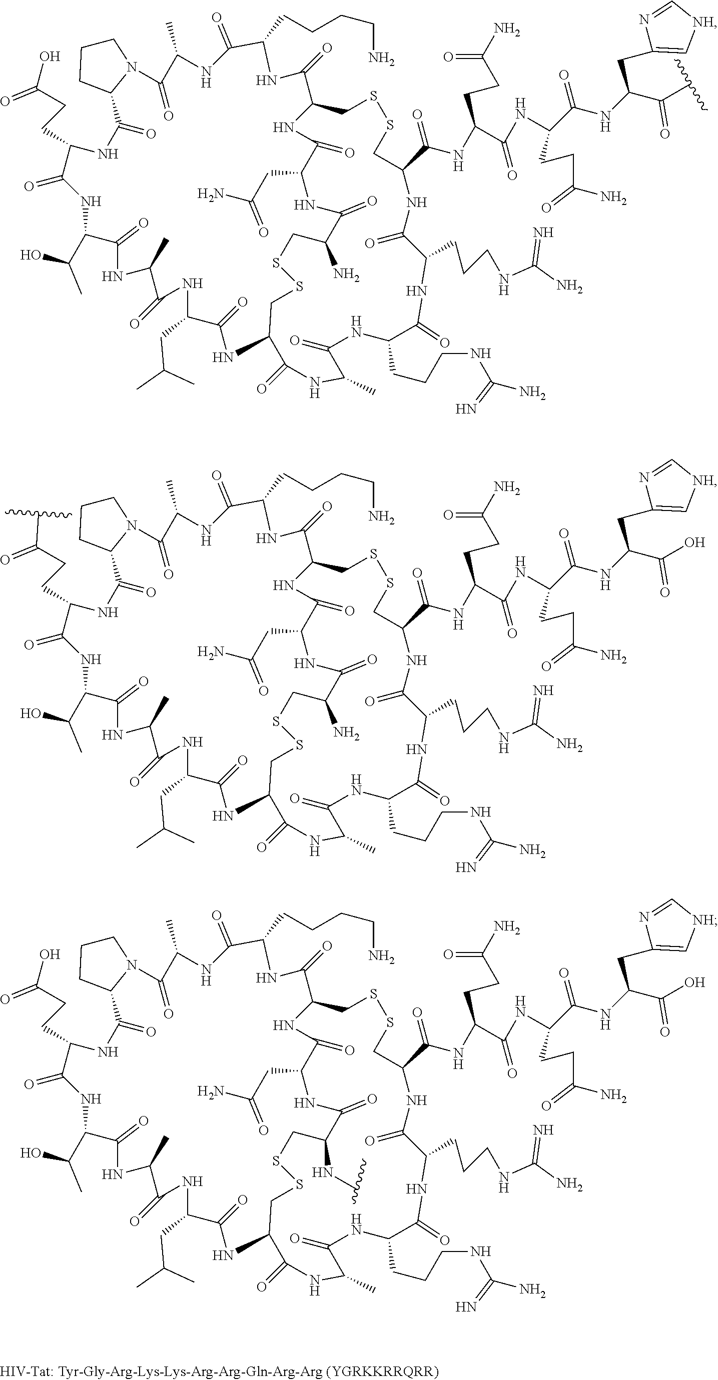

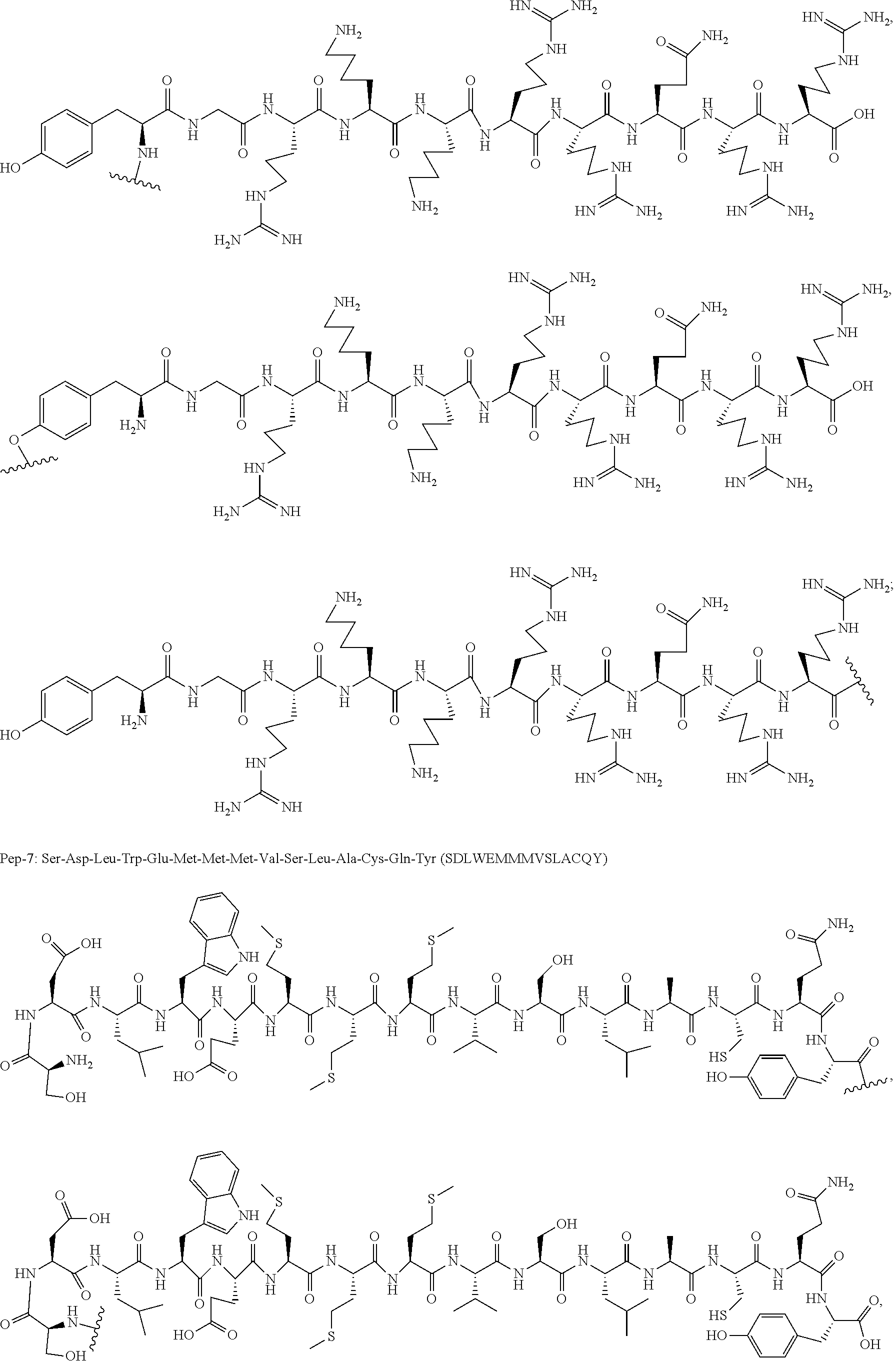

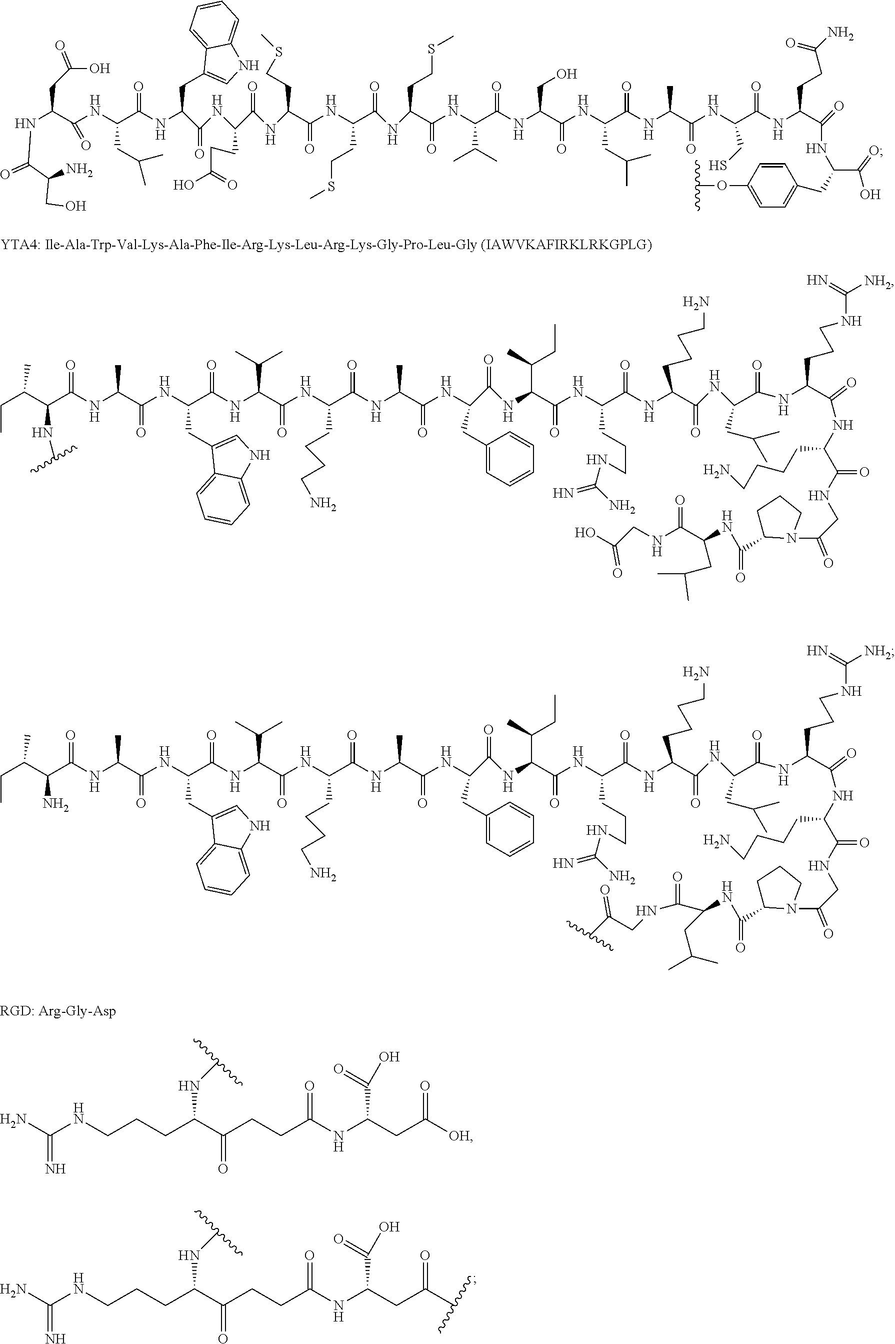

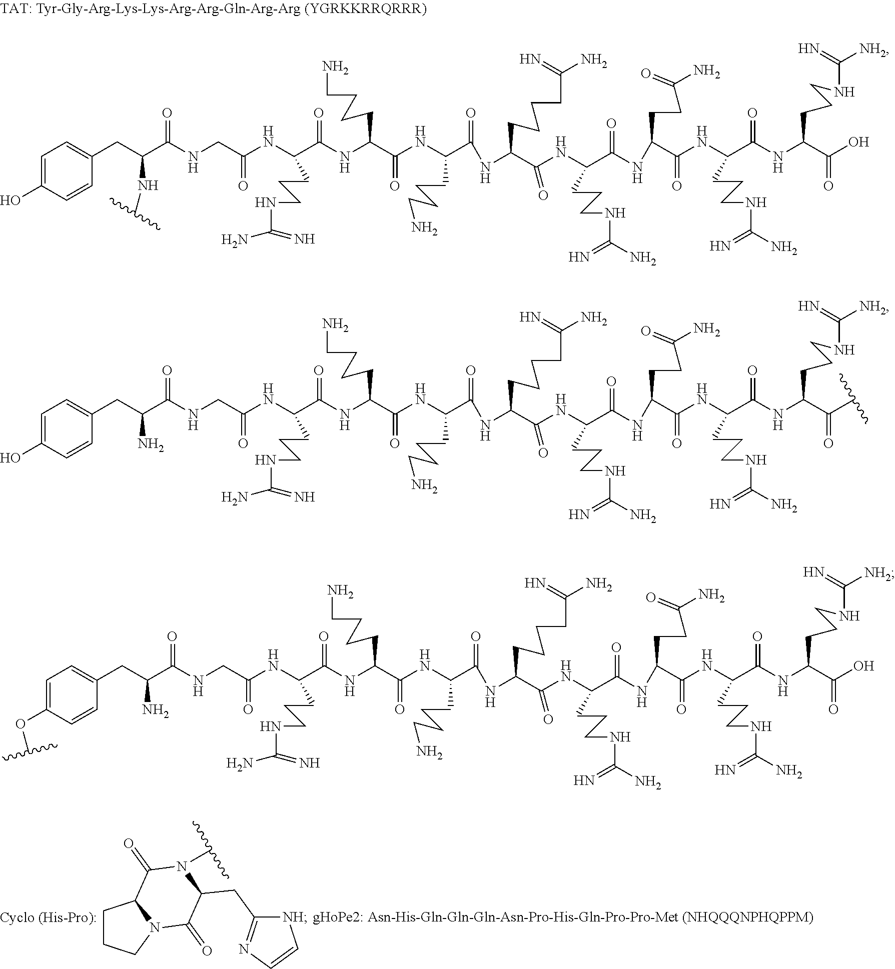

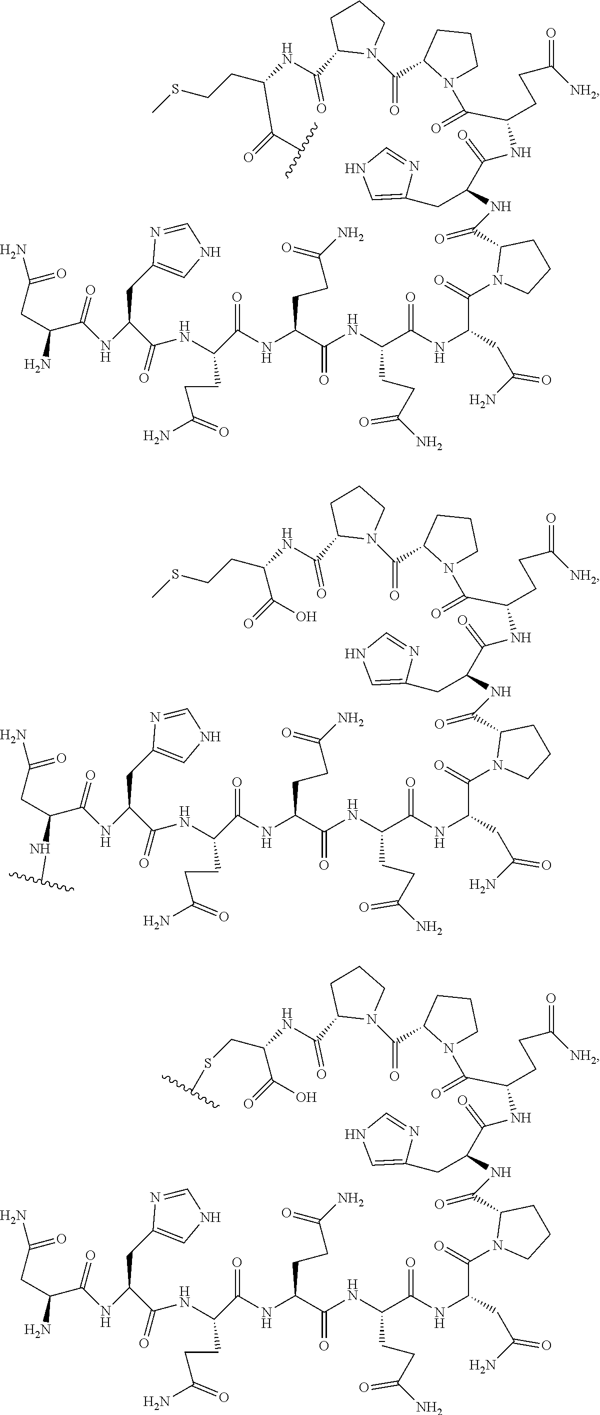

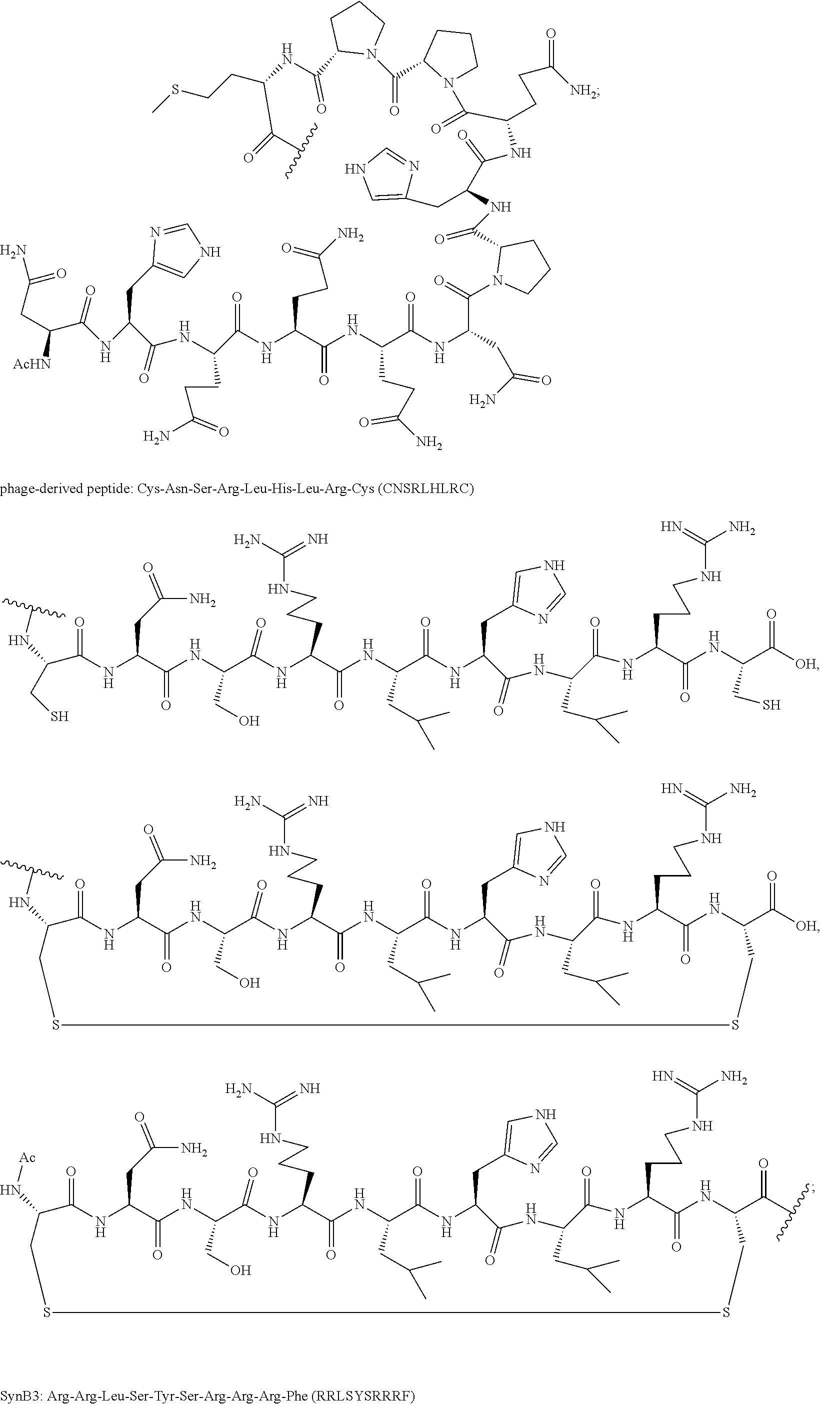

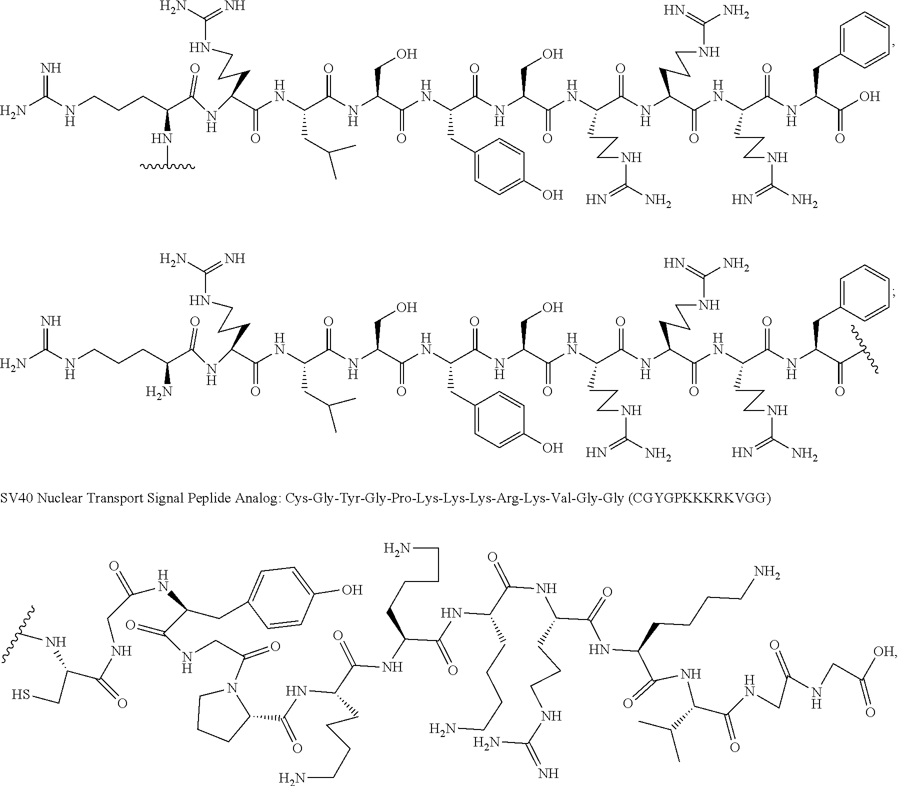

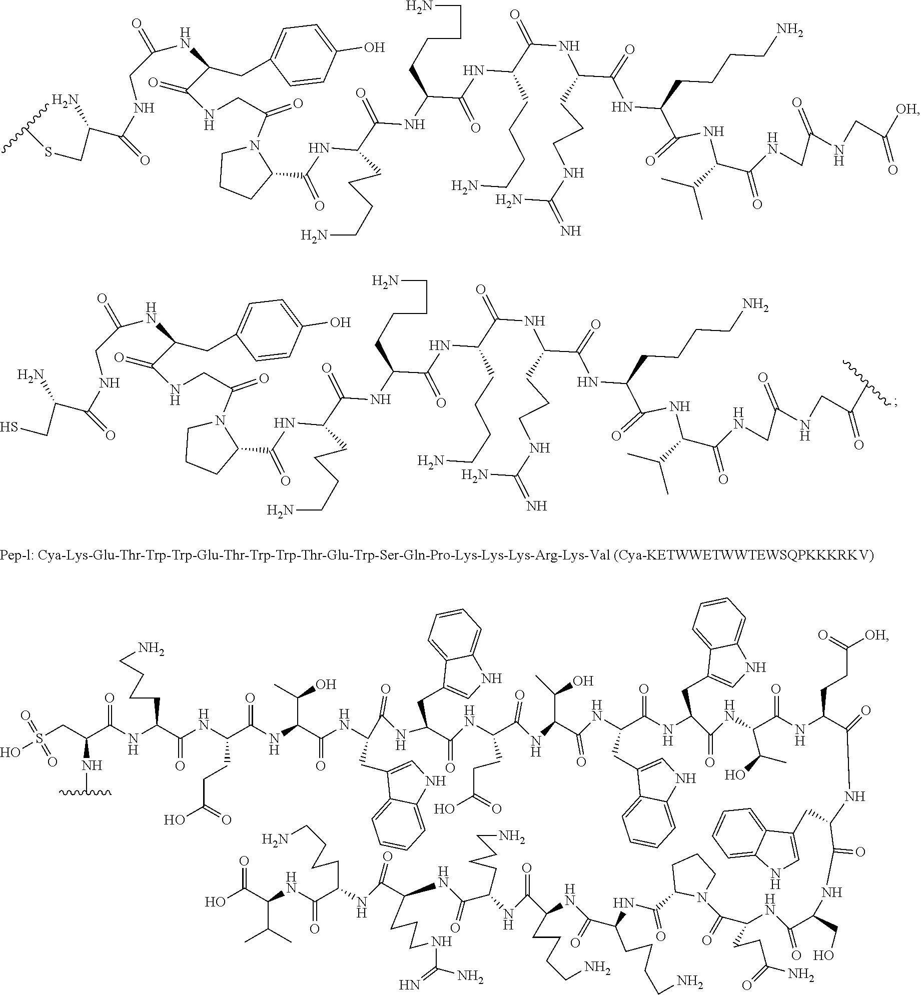

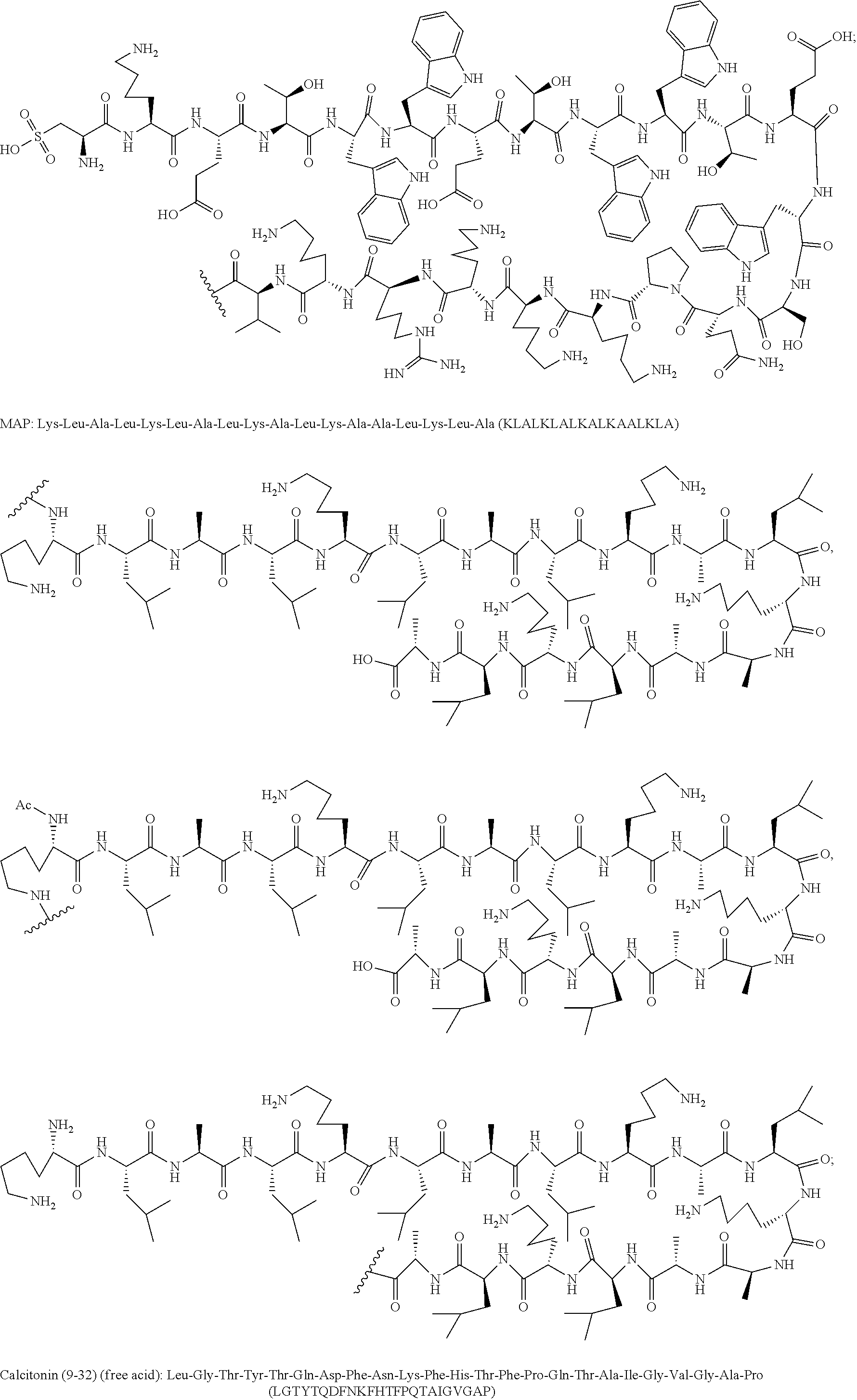

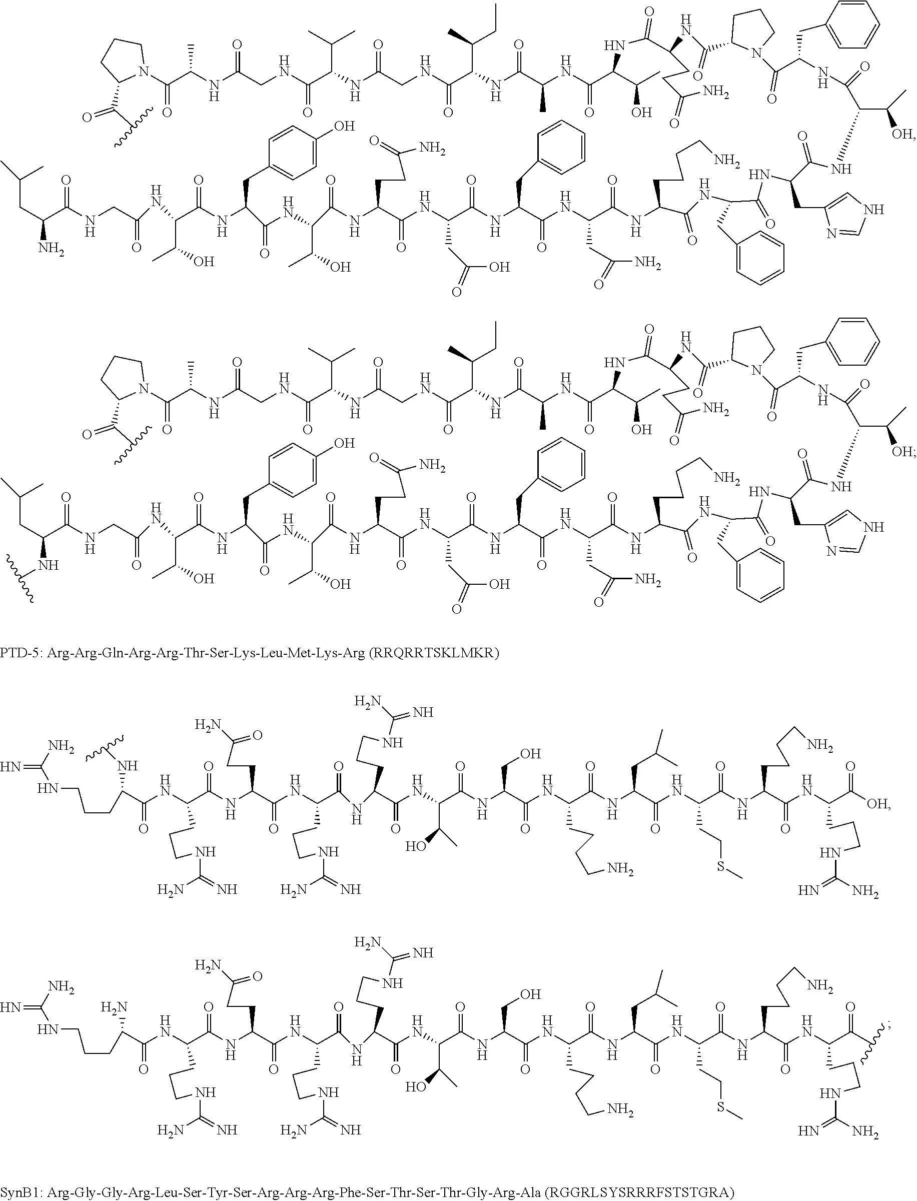

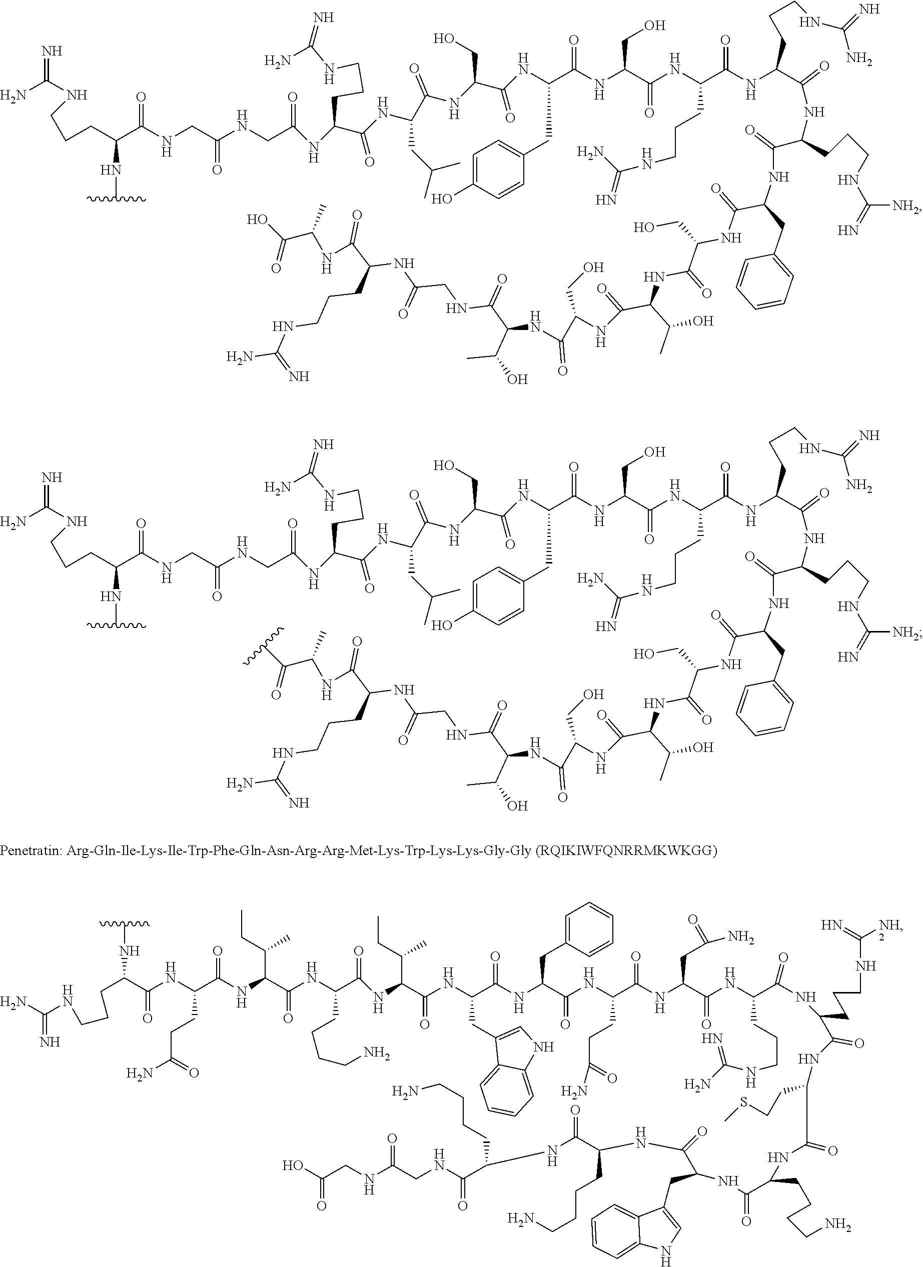

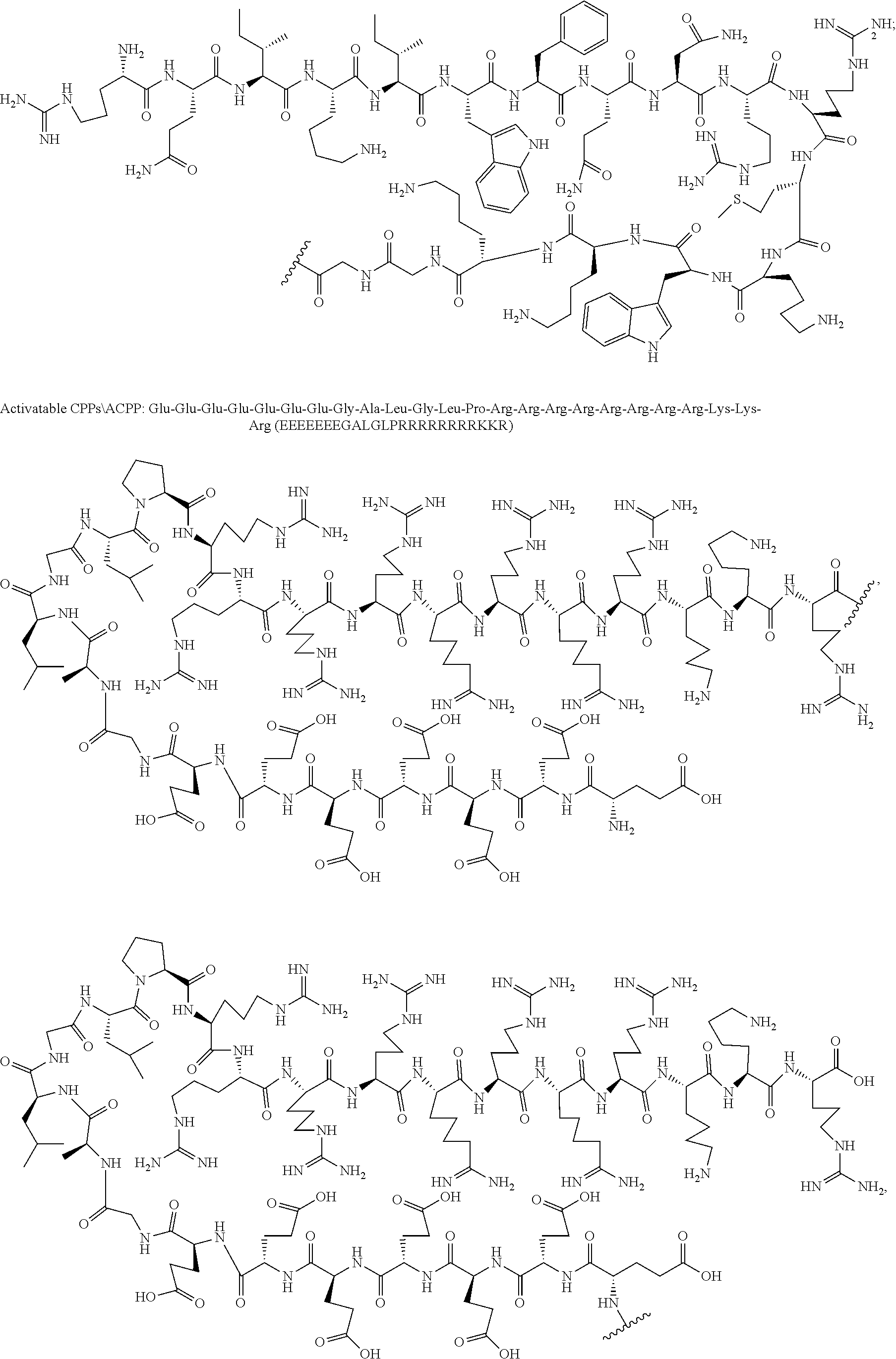

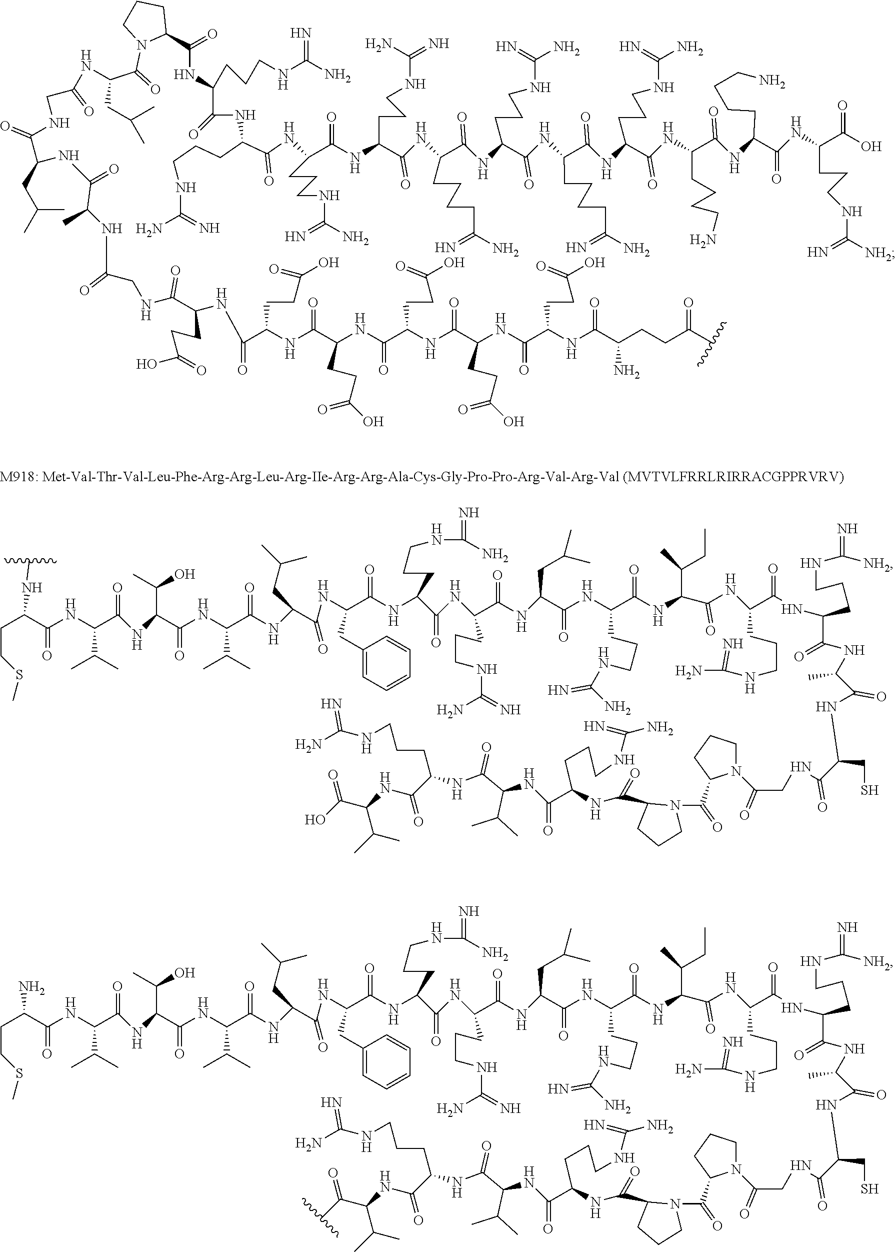

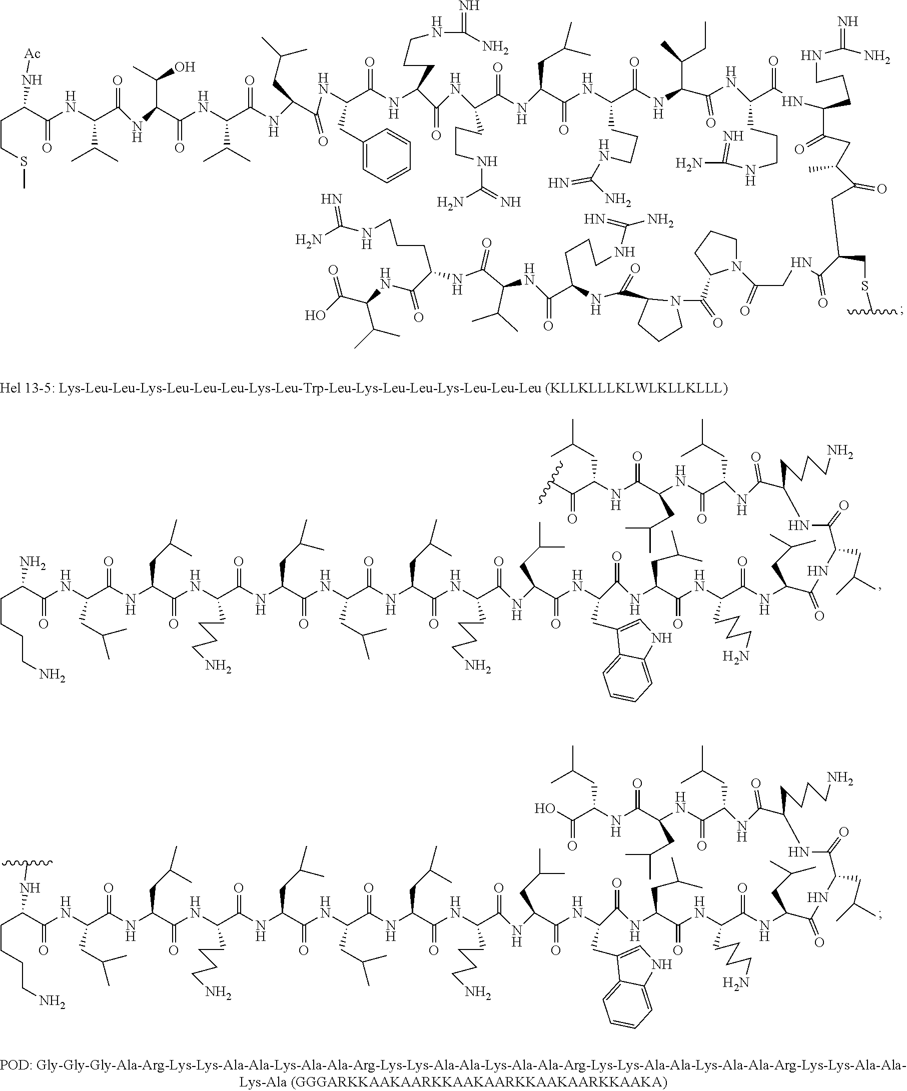

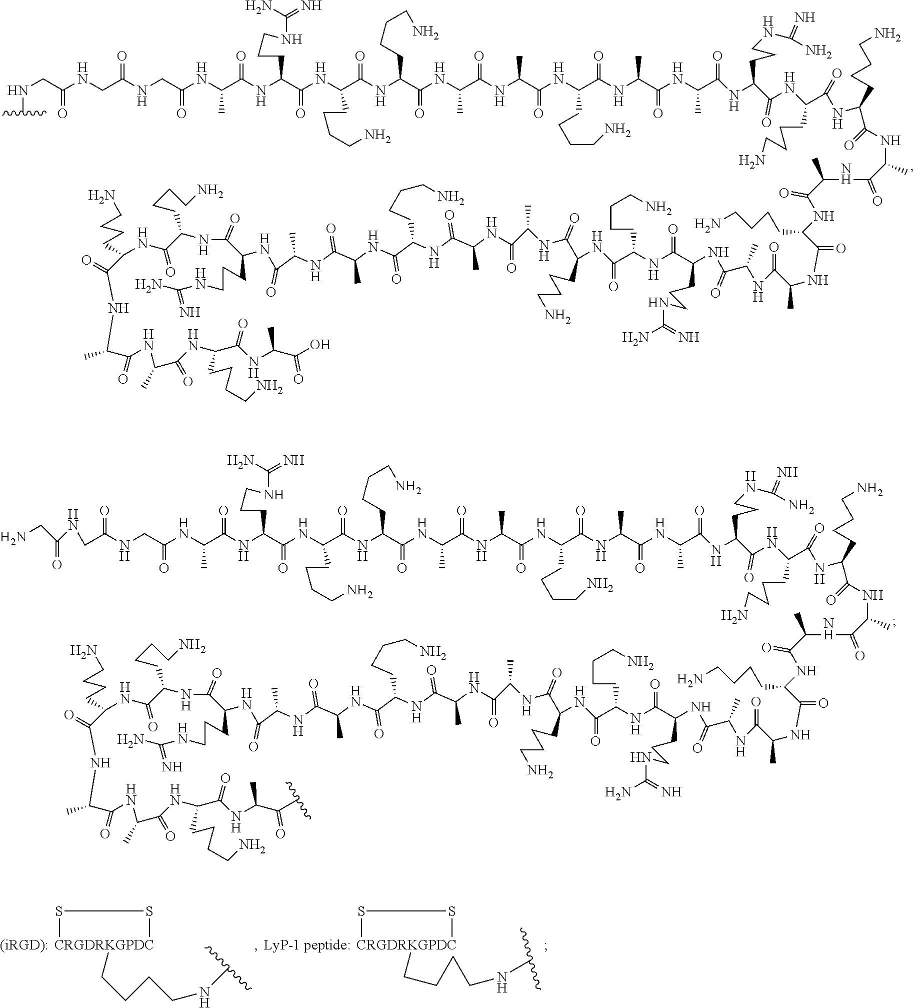

wherein [symbol] is the site linked to Ld₁, Ld₂, Ld₃, Ld₄, Ld₅, or Ld₆; Ra is Ar, which is preferably selected from

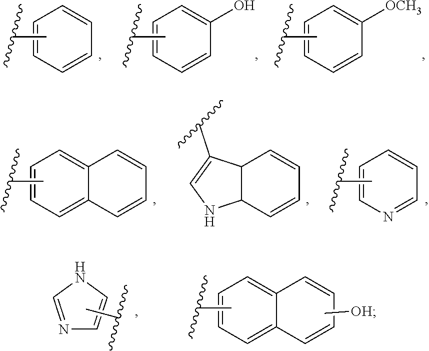

Rb is OH, COOH, COOCH₃, CH₃OH, CH₃NH₂, CONH₂;

In another embodiments, A₁, A₂, A₃, A₄, A₅ and A₆ are preferably Nucleoside analogues, which have synergy with D₁ or/and D₂. The nucleoside analogues are molecules that act like nucleosides in DNA synthesis. They include a range of antiviral products used to prevent viral replication in infected cells. Nucleoside analogues can be used against hepatitis B virus, hepatitis C virus, herpes simplex, and HIV. Once they are phosphorylated, they work as antimetabolites by being similar enough to nucleotides to be incorporated into growing DNA strands. Less selective nucleoside analogues are used as chemotherapy agents to treat cancer, e.g. gemcitabine and 5-FU. Antimetabolite is a chemical that inhibits the use of a metabolite, which is another chemical that is part of normal metabolism. Such substances are often similar in structure to the metabolite that they interfere with, such as the antifolates that interfere with the use of folic acid. The presence of antimetabolites can have toxic effects on cells, such as halting cell growth and cell division, so these compounds are used as chemotherapy for cancer. Some conjugatable structures of nucleoside analogues for this invention are illustrated below:

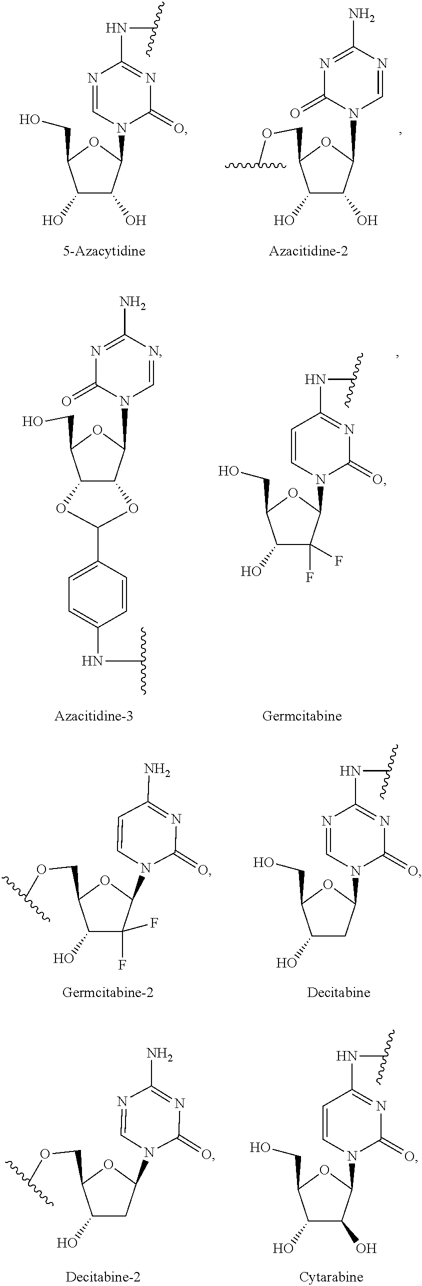

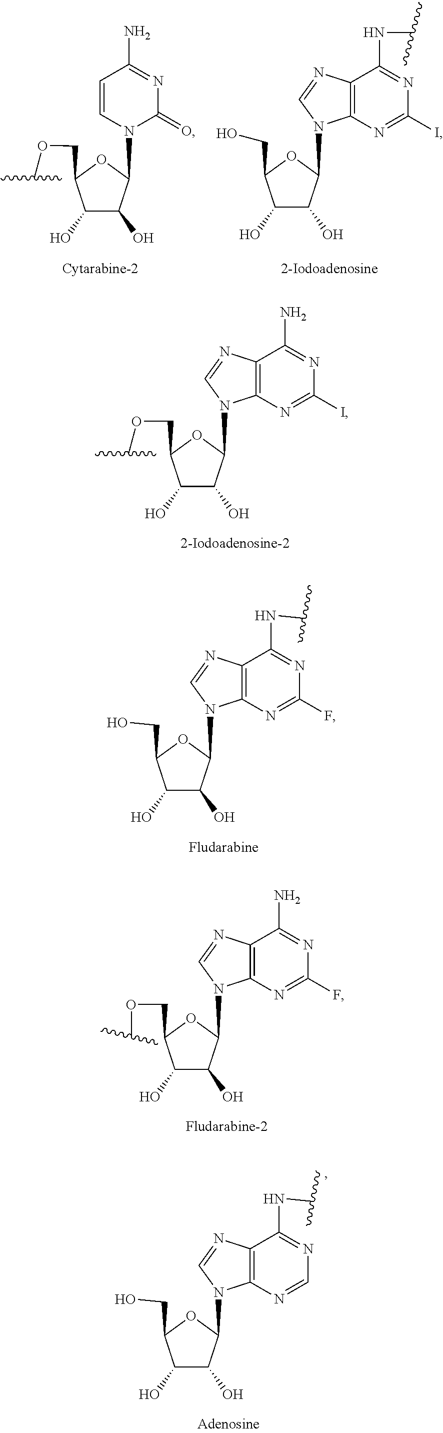

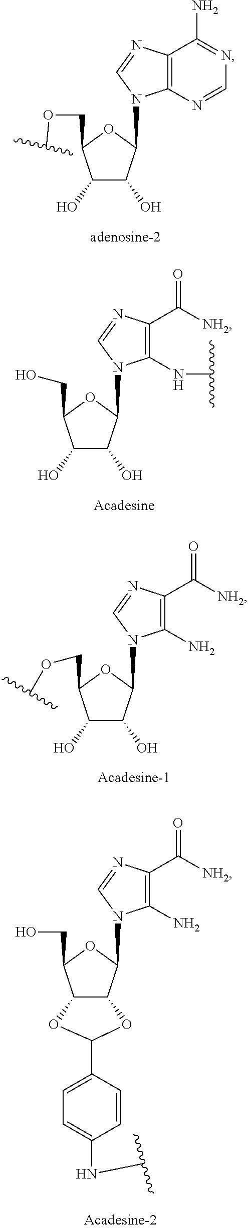

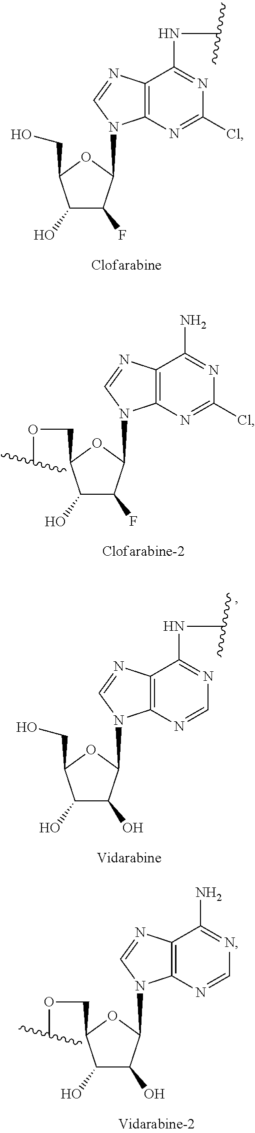 [SMILES: *Nc1nc(Cl)nc2c1ncn2[C@@H]1O[C@H](CO)[C@@H](O)[C@@H]1F ; *OC[C@H]1O[C@@H](n2cnc3c(N)nc(Cl)nc32)[C@@H](F)[C@@H]1O ; *Nc1ncnc2c1ncn2[C@@H]1O[C@H](CO)[C@@H](O)[C@@H]1O ; *OC[C@H]1O[C@@H](n2cnc3c(N)ncnc32)[C@@H](O)[C@@H]1O]

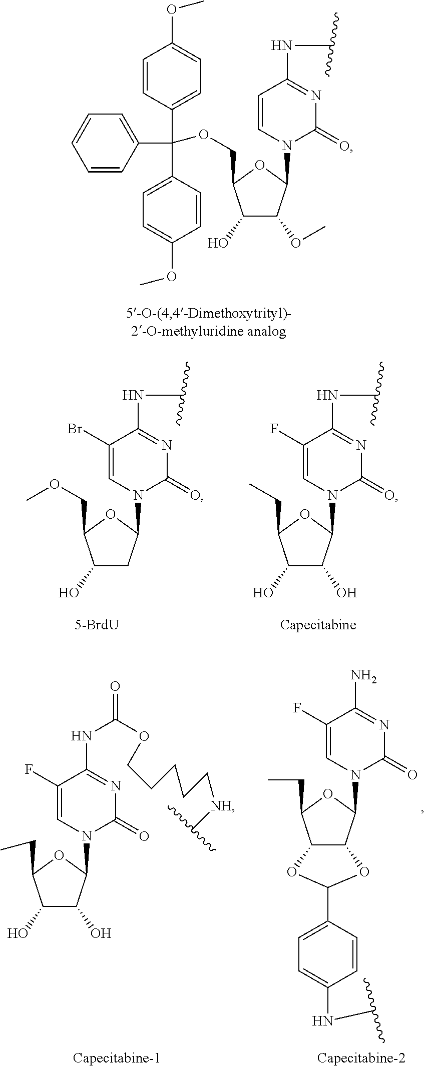

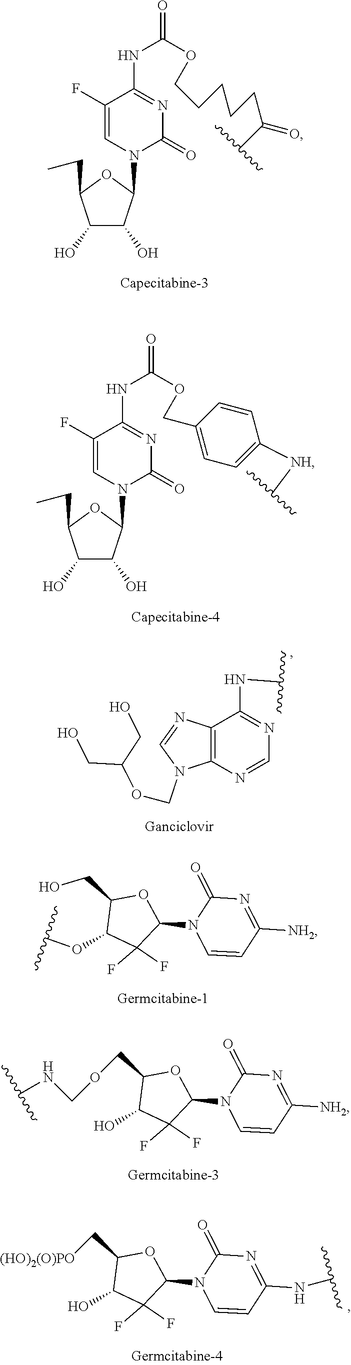

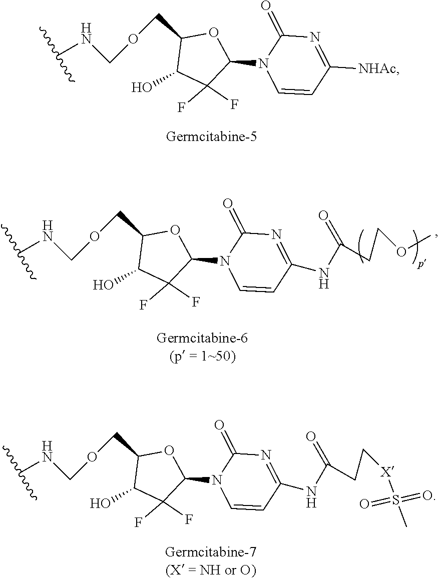

### the Antibody

The antibody (mAb) used for the conjugation process is preferred a cell-binding antibody or antibody-like protein molecule that binds to, complexes with, or reacts with a moiety of a cell population sought to be therapeutically or otherwise biologically modified.

For convenience in this section and elsewhere, “antibody” should be understood to include “antibody-like protein and peptide” except where the context requires otherwise. Suitable antibody-like proteins which may be present in the conjugates of the invention include for example peptides, polypeptides, antibodies, antibody fragments, enzymes, cytokines, chemokines, receptors, blood factors, peptide hormones, toxin, transcription antibody-like proteins, or multimeric antibody-like proteins, wherein they have interchain disulfide bonds structurally.

Enzymes include carbohydrate-specific enzymes, proteolytic enzymes and the like, for example the oxidoreductases, transferases, hydrolases, lyases, isomerases and ligases disclosed by U.S. Pat. No. 4,179,337. Specific enzymes of interest include asparaginase, arginase, adenosine deaminase, superoxide dismutase, catalase, chymotrypsin, lipase, uricase, bilirubin oxidase, glucose oxidase, glucuronidase, galactosidase, glucocerebrosidase, and glutaminase.

Blood antibody-like proteins include albumin, transferrin, Factor VII, Factor VIII or Factor IX, von Willebrand factor, insulin, ACTH, glucagen, somatostatin, somatotropins, thymosin, parathyroid hormone, pigmentary hormones, somatomedins, erythropoietin, luteinizing hormone, hypothalamic releasing factors, antidiuretic hormones, prolactin, interleukins, interferons, for example IFN-α. or IFN-β, colony stimulating factors, haemoglobin, cytokines, antibodies, antibody fragments, chorionicgonadotropin, follicle-stimulating hormone, thyroid stimulating hormone and tissue plasminogen activator.

Other antibody-like proteins of interest are allergen antibody-like proteins disclosed by Dreborg et al Crit. Rev. Therap. Drug Carrier Syst. (1990) 6 315-365 as having reduced allergenicity when conjugated with a polymer such as poly(alkylene oxide) and consequently are suitable for use as tolerance inducers. Among the allergens disclosed are Ragweed antigen E, honeybee venom, mite allergen and the like.

Glycopolypeptides such as immunoglobulins, ovalbumin, lipase, glucocerebrosidase, lectins, tissue plasminogen activator and glycosylated interleukins, interferons and colony stimulating factors are of interest, as are immunoglobulins such as IgG, IgE, IgM, IgA, IgD and fragments thereof. Of particular interest are receptor and ligand binding antibody-like proteins and antibodies and antibody fragments which are used in clinical medicine for diagnostic and therapeutic purposes.

The antibody herein is preferred (A): the group consisting of an antibody, an antibody-like protein molecule, probody, nanobody, peptides, an antibody coating on polymeric micelle, an antibody-liposome, a lipoprotein-based drug carrier, an antibody coating on nano-particle, an antibody-dendrimer, and a particle said above coated or linked with an antibody-like protein (antibody), or a combination of said above thereof;
(B): an antibody, full-length antibodies (polyclonal antibodies, monoclonal antibodies, dimers, multimers, multispecific antibodies (e.g., bispecific antibody, trispecific antibody, or tetraspecific antibody); single chain antibodies; an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune antibody-like proteins (SIP), a lymphokine antibody-like protein, a hormone type antibody-like protein, a growth factor antibody-like protein, a colony stimulating factor antibody-like protein, a nutrient-transport antibody-like protein, large molecular weight antibody-like proteins, fusion antibody-like proteins, a kinase inhibitor antibody-like protein, gene-targeting antibody-like protein, antibody-like protein coated on nanoparticles or polymers modified with antibodies or large molecular weight antibody-like proteins;
The fragments of antibodies include Fab, Fab′, F(ab′)2, Fv, [Parham, J. Immunol. 131, 2895-902 (1983)], fragments produced by a Fab expression library, and epitope-binding fragments of any of the above which immuno-specifically bind to cancer cell antigens, viral antigens, microbial antigens or an antibody-like protein generated by the immune system that is capable of recognizing, binding to a specific antigen or exhibiting the desired biological activity (Miller et al (2003) J. of Immunology 170:4854-61); interferons (such as type I, II, III); peptides; lymphokines such as IL-2, IL-3, IL-4, IL-5, IL-6, IL-10, GM-CSF, interferon-gamma (IFN-Y); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight antibody-like proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Köhler, G.; Milstein, C. (1975). Nature 256:495-7). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0˜20 mM glutamine, 0˜20% fetal calf serum, several ppm amount of heavy metals, such as Cu, Mn, Fe, or Zn, etc, or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80:4949-53 (1983); Geysen et al., Proc. Natl. Acad. Sci. USA, 82:178-82 (1985); Lei et al. Biochemistry 34 (20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352:264-8 (1991); Huse et al., Science 246:1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222:9-27 (2008); Almagro et al, Front Biosci. 13:1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26:39-60 (2004); Houdebine, Curr Opin Biotechnol. 13:625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3:964-70, (2002)); Adams et al, J Immunol Methods. 231:249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, an antibody like peptide or protein that bind/block/target or in some other way interact with the epitopes or corresponding receptors on a targeted cell can be used as a binding molecule. These antibody-like peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of antibody like peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs of this prevention for treating cancer, autoimmune disease, and/or infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (a chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R₂), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α4), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R a), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C₄, anti-HER2/neu), Pexelizumab (anti-C₅), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R₂), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRaβ (T cell receptor alpha/beta), complex, from MedImmune Inc for Graft-versus-host disease], RING SCAN [anti-TAG 72 (tumor associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C₁₁ (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 or DLL4 (delta-like-3 or delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers),

IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MARTI (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumor associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54 (3), 187-207 (2005). Franke, et al, Cancer Biother Radiopharm. 2000, 15, 459-76).

The antibody-like protein, more preferred an IgG antibody that is able to against tumor cells, virus infected cells, microorganism infected cells, parasite infected cells, autoimmune disease cells, activated tumor cells, myeloid cells, activated T-cells, an affecting B cells, or melanocytes. More specifically the antibody is able to against abnormal cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a,b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C₅, C₂₄₂ antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4), CCR5, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSFIR (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C-X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRVIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α6β4, α7β7, αllβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation-inhibiting factor (GIF)), MS4A1 (membrane-spanning 4-domains subfamily A member 1), B7H3, B7H4, MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page 4, PAP, Paratope of anti-(N-glycolylneuraminic acid), PAX3, PAX5, PCSK9, PDCD1 (PD-1, Programmed cell death protein 1), PDGF-Rα (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-1 (Trop1), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

Many of tumor-associated antigens (TAA) or tumor cell receptors are known in the art, and can be prepared for use in generating antibodies using methods and information which are well known in the art. In attempts to discover effective cellular targets for cancer diagnosis and therapy, researchers have sought to identify transmembrane or otherwise tumor-associated polypeptides or glycoproteins that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via the ADCs of this application. Examples of the TAA and cognate antibodies with their known in art are:
(1) BMPR1B (Bone Morphogenetic Protein Receptor-Type IB);
(2) E16 (LATI, SLC7A₅);
(3) STEAP1 (Six Transmembrane Epithelial Antigen of Prostate),
(4) 0772P (CA125, MUC16);
(5) MPF (MPF, MSLN, SMR, Megakaryocyte Potentiating Factor, Mesothelin);
(6) Napi3b (NAPI-3B, NPTIIb, SLC34A2, Solute Carrier Family 34 (Sodium Phosphate), Member 2, Type II Sodium-Dependent Phosphate Transporter 3b);
(7) Sema 5b (FLJ10372, KIAA1445, Mm.42015, SEMA5B, SEMAG, Semaphorin 5b Hlog, 25 Sema Domain, Seven Thrombospondin Repeats (Type 1 and Type 1-Like), Transmembrane Domain™ and Short Cytoplasmic Domain, (Semaphorin) 5B);
(8) PSCA hlg (2700050C12Rik, C530008016Rik, RIKEN cDNA 2700050C₁₂, RIKEN cDNA 2700050C12 gene);
(9) ETBR (Endothelin Type B Receptor);
(10) MSG783 (RNF124, Hypothetical Protein FLJ20315);
(11) STEAP2 (HGNC_8639, IPCA-1, PCANAP1, STAMP1, STEAP2, STMP, Prostate Cancer Associated Gene 1, Prostate Cancer Associated Protein 1, Six Transmembrane Epithelial Antigen of Prostate 2, Six Transmembrane Prostate Protein);
(12) TrpM4 (BR22450, FLJ20041, TRPM4, TRPM4B, Transient Receptor Potential Cation 5 Channel, Subfamily M, Member 4);
(13) CRIPTO (CR, CR1, CRGF, CRIPTO, TDGF1, Teratocarcinoma-Derived Growth Factor);
(14) CD21 (CR2 (Complement Receptor 2) or C₃DR (C₃d/Epstein Barr Virus Receptor) or Hs. 73792);
(15) CD79b (CD79B, CD793, IGb (Immunoglobulin-Associated Beta), B29);
(16) FcRH2 (IFGP4, IRTA4, SPAP1A (SH2 Domain Containing Phosphatase Anchor Protein 5 La), SPAP1B, SPAP1C);
(17) HER2 (ErbB2);
(18) NCA (CEACAM6);
(19) MDP (DPEP1);
(20) IL20R-Alpha (IL20Ra, ZCYTOR7);
(21) Brevican (BCAN, BEHAB);
(22) EphB2R (DRT, ERK, Hek5, EPHT3, Tyro5);
(23) ASLG659 (B7h);
(24) PSCA (Prostate Stem Cell Antigen Precursor);
(25) GEDA;
(26) BAFF-R (B Cell-Activating Factor Receptor, BLyS Receptor 3, BR3);
(27) CD22 (B-Cell Receptor CD22-B Isoform, BL-CAM, Lyb-8, Lyb8, SIGLEC-2, FLJ22814); (27A) Cd22 (Cd22 Molecule);
(28) CD79a (CD79A, CD79alpha), Immunoglobulin-Associated Alpha, a B Cell-Specific Protein that Covalently Interacts with Ig Beta (CD79B) and Forms a Complex on the Surface with Ig M 35 Molecules, Transduces a Signal Involved in B-Cell Differentiation), Pl: 4.84, MW: 25028 Tm: 2 [P] Gene Chromosome: 19q13.2);
(29) CXCR5 (Burkitt's Lymphoma Receptor 1, a G Protein-Coupled Receptor that is Activated by the CXCL13 Chemokine, Functions in Lymphocyte Migration and Humoral Defense, Plays a 10 Role in HIV-2 Infection and Perhaps Development of AIDS, Lymphoma, Myeloma, and Leukemia); 372 Aa, Pl: 8.54 MW: 41959 TM: 7 [P] Gene Chromosome: 11q23.3;
(30) HLA-DOB (Beta Subunit of MHC Class II Molecule (La Antigen) that Binds Peptides and 20 Presents them to CD4+T Lymphocytes); 273 Aa, Pl: 6.56, MW: 30820. TM: 1 [P] Gene Chromosome: 6p21.3);
(31) P2X₅ (Purinergic Receptor P2X Ligand-Gated Ion Channel 5, an Ion Channel Gated by Extracellular ATP, May be Involved in Synaptic Transmission and Neurogenesis, Deficiency May Contribute to the Pathophysiology of Idiopathic Detrusor Instability); 422 Aa), Pl: 7.63, MW: 47206 TM: 1 [P] Gene Chromosome: 17p13.3);
(32) CD72 (B-Cell Differentiation Antigen CD72, Lyb-2); 359 Aa, Pl: 8.66, MW: 40225, TM: 1 5 [P] Gene Chromosome: 9p13.3);
(33) LY64 (Lymphocyte Antigen 64 (RP105), Type I Membrane Protein of the Leucine Rich Repeat (LRR) Family, Regulates B-Cell Activation and Apoptosis, Loss of Function is Associated with Increased Disease Activity in Patients with Systemic Lupus Erythematosis); 661 Aa, pl: 6.20, MW: 74147 TM: 1 [P] Gene Chromosome: 5q12);
(34) FcRHI (Fc Receptor-Like Protein 1, a Putative Receptor for the Immunoglobulin Fc Domain that Contains C2 Type Ig-Like and ITAM Domains, May have a Role in B-Lymphocyte 20 Differentiation); 429 Aa, Pl: 5.28, MW: 46925 TM: 1 [P] Gene Chromosome: 1q21-1q22);
(35) IRTA2 (Immunoglobulin Superfamily Receptor Translocation Associated 2, a Putative Immunoreceptor with Possible Roles in B Cell Development and Lymphomagenesis; Deregulation of the Gene by Translocation Occurs in Some B Cell Malignancies); 977 Aa, pl: 6.88, MW: 106468, TM: 1 [P] Gene Chromosome: 1q21);
(36) TENB2 (TMEFF2, Tomoregulin, TPEF, HPP1, TR, Putative Transmembrane 35 Proteoglycan, Related to the EGF/Heregulin Family of Growth Factors and Follistatin); 374 Aa);
(37) PSMA-FOLHI (Folate Hydrolase (Prostate-Specific Membrane Antigen) 1);
(38) SST (Somatostatin Receptor; Note that there are 5 Subtypes);
(38.1) SSTR2 (Somatostatin Receptor 2);
(38.2) SSTR5 (Somatostatin Receptor 5);
(38.3) SSTR1; (38.4) SSTR3; (38.5) SSTR4; AvB6—Both Subunits (39+40);
(39) ITGAV (Integrin, Alpha V);
(40) ITGB6 (Integrin, Beta 6);
(41) CEACAM5 (Carcinoembryonic Antigen-Related Cell Adhesion Molecule 5);
(42) MET (Met Proto-Oncogene; Hepatocyte Growth Factor Receptor);
(43) MUC1 (Mucin 1, Cell Surface Associated);
(44) CA9 (Carbonic Anhydrase IX);
(45) EGFRvIII (Epidermal Growth Factor Receptor (EGFR), Transcript Variant 3);
(46) CD33 (Cd33 Molecule);
(47) CD19 (Cd19 Molecule):
(48) IL2RA (Interleukin 2 Receptor, Alpha); NCBI Reference Sequence: NM_000417.2);
(49) AXL (AXL Receptor Tyrosine Kinase);
(50) CD30-TNFRSF8 (Tumor Necrosis Factor Receptor Superfamily, Member 8);
(51) BCMA (B-Cell Maturation Antigen)-TNFRSF17 (Tumor Necrosis Factor Receptor Superfamily, Member 17);
(52) CT Ags-CTA (Cancer Testis Antigens);
(53) CD174 (Lewis Y)-FUT3 (Fucosyltransferase 3 (Galactoside 3 (4)-L-Fucosyltransferase, Lewis Blood Group);
(54) CLEC14A (C-Type Lectin Domain Family 14, Member a; Genbank Accession No. NM175060);
(55) GRP78-HSPA5 (Heat Shock 70 kDa Protein 5 (Glucose-Regulated Protein, 78 kDa);
(56) CD70 (Cd70 Molecule) L08096;
(57) Stem Cell Specific Antigens: For Example: 5T4 (see entry (63) below); CD25 (see entry (48) above);
(58) ASG-5;
(59) ENPP3 (Ectonucleotide Pyrophosphatase/Phosphodiesterase 3);
(60) PRR4 (Proline Rich 4 (Lacrimal));
(61) GCC-GUCY2C (Guanylate Cyclase 2C (Heat Stable Enterotoxin Receptor);
(62) Liv-1-SLC39A6 (Solute Carrier Family 39 (Zinc Transporter), Member 6);
(63) 5T4, Trophoblast Glycoprotein, TPBG-TPBG (Trophoblast Glycoprotein);
(64) CD56-NCMA 1 (Neural Cell Adhesion Molecule 1);
(65) CanAg (Tumor Associated Antigen CA242);
(66) FOLR1 (Folate Receptor 1);
(67) GPNMB (Glycoprotein (Transmembrane) Nmb);
(68) TIM-1—HAVCR1 (Hepatitis a Virus Cellular Receptor 1);
(69) RG-1/Prostate Tumor Target Mindin—Mindin/RG-1;
(70) B7-H4—VTCN1 (V-Set Domain Containing T Cell Activation Inhibitor 1;
(71) PTK7 (PTK7 Protein Tyrosine Kinase 7);
(72) CD37 (Cd37 Molecule);
(73) CD138—SDC1 (Syndecan 1);
(74) CD74 (CD74 Molecule, Major Histocompatibility Complex, Class II Invariant Chain);
(75) Claudins—CLs (Claudins);
(76) EGFR (Epidermal Growth Factor Receptor);
(77) Her3 (ErbB3)-ERBB3 (v-Erb-b2 Erythroblastic Leukemia Viral Oncogene Homolog 3 (Avian));
(78) RON—MSTIR (Macrophage Stimulating 1 Receptor (c-Met-Related Tyrosine Kinase));
(79) EPHA2 (EPH Receptor A₂);
(80) CD20—MS4A1 (Membrane-Spanning 4-Domains, Subfamily a, Member 1); (81) Tenascin C—TNC (Tenascin C);
(82) FAP (Fibroblast Activation Protein, Alpha);
(83) DKK-1 (Dickkopf 1 Homolog (Xenopus laevis);
(84) CD52 (Cd52 Molecule);
(85) CS1—SLAMF7 (SLAM Family Member 7);
(86) Endoglin-ENG (Endoglin);
(87) PMEL17 (silver homolog; SILV; D12S53E; PMEL17; SI; SIL); ME20; gp100) BC001414; BT007202; M32295; M77348; NM_006928;
(88) TMEFF1 (transmembrane protein with EGF-like and two follistatin-like domains 1; Tomoregulin-1); H7365; C9orf2; C90RF2; U19878; X83961; NM_080655; NM_003692;
(89) GDNF-Ral (GDNF family receptor alpha 1; GFRA1; GDNFR; GDNFRA; RETLi; TRNR1; RETIL; GDNFR-alpha1; GFR-ALPHA-1); U95847; BC014962; NM 145793 NM_005264;
(90) Ly6E (lymphocyte antigen 6 complex, locus E; Ly67, RIG-E, SCA-2, TSA-1); NP_002337.1; NM_002346.2;
(91) TMEM46 (shisa homolog 2 (Xenopus laevis); SHISA2); NP_001007539.1; NM_001007538.1;
(92) Ly6G6D (lymphocyte antigen 6 complex, locus G6D; Ly6-D, MEGT1); NP_067079.2; NM 021246.2;
(93) LGR5 (leucine-rich repeat-containing G protein-coupled receptor 5; GPR49, GPR67); NP_003658.1; NM_003667.2;
(94) RET (ret proto-oncogene; MEN2A; HSCR1; MEN2B; MTC1; PTC; CDHF12; Hs. 168114; RET51; RET-ELE1); NP_066124.1; NM_020975.4;
(95) LY6K (lymphocyte antigen 6 complex, locus K; LY6K; HSJ001348; FLJ35226); NP_059997.3; NM_017527.3;
(96) GPR19 (G protein-coupled receptor 19; Mm.4787); NP_006134.1; NM_006143.2; (97) GPR54 (KISS1 receptor; KISS1R; GPR54; HOT7T175; AXOR12); NP_115940.2; NM 032551.4;
(98) ASPHD1 (aspartate beta-hydroxylase domain containing 1; LOC253982); NP_859069.2; NM 181718.3;
(99) Tyrosinase (TYR; OCA1A; OCA1A; tyrosinase; SHEP3); NP_000363.1; NM_000372.4;
(100) TMEM118 (ring finger protein, transmembrane 2; RNFT2; FLJ14627); NP_001103373.1; NM 001109903.1;
(101) GPR172A (G protein-coupled receptor 172A; GPCR41; FLJ11856; D₁₅Ertd747e); NP_078807.1; NM_024531.3;
(102) CLL-1 (CLEC12A, MICL, and DCAL2), encodes a member of the C-type lectin/C-type lectin-like domain (CTL/CTLD) superfamily;
(103) Annexin A1-ANXA1 (Annexin A1);
(104) V-CAM (CD106)-VCAM1 (Vascular Cell Adhesion Molecule 1);
(105) B7H₃ (CD276 or B7RP-2, a member of the B7 ligand family, has two isoforms: 2Ig- and 4Ig-B7H₃ with molecular weights of approximately 45-kDa and 100-kDa, respectively);
(106) CD54 (also Known as BB2; CD54; P3.58);
(107) CA19-9 (Carbohydrate antigen 19-9 (CA 19-9) is a cell surface glycoprotein complex most commonly associated with pancreatic ductal adenocarcinoma (PDAC);
(108) Tissue Factor (coagulation factor III, tissue factor, TF; TFA; CD142);
(109) ROR1 (receptor tyrosine kinase like orphan receptor 1, NTRKR1; dJ537F10.1);
(110) Claudin18 (SFTA5; SFTPJ); (111) FGFR2 (fibroblast growth factor receptor 2, BEK; JWS; BBDS; CEK3; CFD1; ECT1; KGFR; TK14; TK25; BFR-1; CD332; K-SAM);
(112) FGFR3 (fibroblast growth factor receptor 3, ACH; CEK2; JTK4; CD333; HSFGFR3EX);
(113) FGFR4 (fibroblast growth factor receptor 4, TKF; JTK2; CD334);
(114) FGFR1 (fibroblast growth factor receptor 1, CEK; FLG; HH2; OGD; ECCL; FLT2; KAL2; BFGFR; CD331; FGFBR; FLT-2; HBGFR; N-SAM; FGFR-1; HRTFDS; bFGF-R-1);
(115) ROR2 (receptor tyrosine kinase like orphan receptor 2, BDB; BDB1; NTRKR2);
(116) SLC44A4 (SLC44A4-solute carrier family 44 member 4, CTL4; NG22; TPPT; DFNA72; hTPPT1; C₆orf29);
(117) DLL3 (delta like canonical Notch ligand 3, SCDO1);
(118) DLL4 (delta like canonical Notch ligand 4, AOS6; delta4; hdelta2);
(119) ALK (ALK receptor tyrosine kinase, ALK1; CD246; NBLST3);
(120) CLDN6 (claudin 6);
(121) CLDN3 (claudin 3, RVP1; HRVP1; C7orf1; CPE-R₂; CPETR2);
(122) EFNA4 (ephrin A4, EFL4; EPLG4; LERK4; LERK-4);
(123) Notch1 (notch receptor 1, hN1; AOS5; TAN1; AOVD1)
(124) Notch2 (notch receptor 2, hN2; AGS2; HJCYS);
(125) Notch3 (notch receptor 3, IMF2; LMNS; CASIL; CADASIL; CADASIL1);
(126) CDH17 (Cadherin 17, or Liver-Intestine-cadherin (LI-cadherin) is a type-I transmembrane glycoprotein that belongs to the 7D-cadherin superfamily;
(127) LAMP-1 (lysosomal associated membrane protein 1, LAMPA; CD107a; LGP120);
Many of the above antigens were described in our previous patent application (PCT/CN2023/096066 field on May 24, 2023).

In another specific embodiment, the antibody-drug conjugates of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproliferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the Myeloproliferative antibody-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenia purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barré syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenia purpura (See Autoimmune thrombocytopenia purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Ménière's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, the antibody-drug conjugates of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub. 1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial (M2) antibody; Rheumatoid factor antibody; Anti-MCV antibody; Anti-topoisomerase antibody; Anti-neutrophil cytoplasmic (cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF-R1, TNFR-2, RANK, TAC1, BCMA, osteoprotegerin, Apo2/TRAIL-R1, TRAIL-R₂, TRAIL-R3, TRAIL-R₄, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimi-dase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PRO542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The antibody-drug conjugates of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The cell binding molecule, which is more preferred to be an antibody described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)-Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157: H₇, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as cell binding ligands used in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

In another embodiment of the present invention, the present invention also provides an antibody-drug conjugate (ADC) comprising a monoclonal antibody, or an antigen-binding fragment thereof, conjugated with a cytotoxin, via a linker containing a chemotherapeutic small molecule, which is selected from an antimetabolite drug: 5-Azacytidine, Azacitidine-2, Azacitidine-3, Germcitabine, Germcitabine-1, Germcitabine-2, Germcitabine-3, Germcitabine-4, Germcitabine-5, Germcitabine-6, Germcitabine-7, Capecitabine, Capecitabine-1, Capecitabine-2, Capecitabine-3, Capecitabine-4, Decitabine, Decitabine-2, Cytarabine, Cytarabine-2, 2-Iodoadenosine, 2-Iodoadenosine-2, Fludarabine, Fludarabine-2, Adenosine, Adenosine-2, Acadesine, Acadesine-1, Acadesine-2, Clofarabine, Clofarabine-2, Vidarabine, Vidarabine-2, 5,O-(4,4′-dimethoxytrityl)-2′-O-methyluridine, 5-BrdU, and Ganciclovir, and their structures are illustrated above.

In further preferred embodiments, the present invention also provides an antibody-drug conjugate (ADC) comprising a monoclonal antibody, or an antigen-binding fragment thereof, conjugated with a cytotoxin, via a linker containing a glutamate urea small molecule, such as 2-[3-(1,3-dicarboxypropyl) ureido]-pentanedioic acid (DUPA), urea-based glutamate heterodimers, 2-(phosphonomethyl)-pentanedioic acid (PMPA), phosphoramidates, glu-urea-lys, or 2-(phosphinylmethyl) pentanedioic acids analog group to direct against prostate antigen (PSA) of a tumor cell, and/or an affinity ligand for bombesin receptors (Gastrin releasing peptide receptor (GRPR), neurotensin receptors (including Neurotensin receptor 1 (NTR1) and neuropeptide-Y receptors), and/or a cell-penetrating peptide, and/or an affinity peptide that can bind with a protein called programmed death ligand-1 (PD-L1, or CD274) which is expressed on tumor cells and tumor-infiltrating immune cells, blocking its interactions with both PD-1 and B7.1 receptors. The affinity to the receptors are at least EC₅₀<10 μM, preferably EC₅₀<100 nM, and more preferably EC50<50 nM. In a further embodiment the antigen binding proteins are conjugated to a cytotoxin, such as, but not limited, a tubulysin analog, a camptothecin (CPT) analog, a PBD dimer, an anthracycline, or an auristatin analog.

In some embodiments, the cell-penetrating peptide (CPP) used in this invention can be seleted from CPP database (http://crdd.osdd.net/raghava/cppsite) or from known publications with less than 100 amino acids of sequences or from amendment of known peptide sequences with replacement of one or several amino acids, and then is subjected to redundancy check. The preferred CPP is a linear or cyclo-peptide having less than 50 amino acids, preferably less than 20 natural or unnatural amino acids, more preferably less than 15 amino acids and containing one, two, or several arginines and/or lysines. The CPP is more preferably a cyclopeptide, in particular CPP is a cyclopeptide having less than 8 amino acids. The selected peptides are normally further analyzed to filter out the ambiguous peptides with undesirable chemical modifications. The amphipathicity prediction can be through the online server AMPHIPASEEK (https://npsa-prabi.ibcp.fr/cgibin/npsa_automat.pl?page=/NPSA/npsa_amphipaseek.html). AMPHIPASEEK provides a score for every residue between a range of 0 and 5 for the given peptide sequences. Higher scores imply high amphipathic nature and vice versa (0=low and 5=high). Hydropathy values were (K-Me3) calculated using the online server (https://www.peptide2.com/N_peptide_hydrophobicity_hydrophilicity.php). The CPPs are normally required to pass through the criteria of peptide solubility and the cell-penetrating property using Innovagen peptide solubility calculator (https://pepcalc.com/) and CPPpred (http://bioware.ucd.ie/˜compass/biowareweb/Server_pages/cpppred.php), respectively. The CPP score is given within the range of 0-1, wherein the peptides with the score of >0.5 are suggestive of better cell penetration. The efficiency of CPP penetration of a cell can be measured in several different methods (Lee, H-M, et al, Nature Communications Biology 2021, 4:205; Penedo, M. et al, Scientific Reports, 2021, 11:7756 and the references they incorporated). In general, the preferable CPP should enable to internalize (trafficking) over 40% of the ligand bound on a cell or help to internalize 40% of ADCs bound on a cell to cross the cell membrane in 2 hours.

In some embodiments, the present invention provides antigen binding antibody-drug conjugates which bind to membrane bound targets and wherein the antigen binding ADC is capable of internalisation. In a further embodiment there is provided an immunoconjugate comprising the antigen binding protein of the present invention and a cytotoxic agent. In a further embodiment the antigen binding protein has ADCC effector function for example the antigen binding protein has enhanced ADCC effector function. In one such embodiment there is provided antigen binding antibodies/proteins or fragments of the antibodies used for ADCs against various cancers thereof.

In one aspect of the invention, the provided an antibody/protein used for the antibody-drug conjugate of this invention is preferably selected from an antibody having affinity to an antigen of highly expressed on tumor cells The information including the sequences of the provided antibody can be found in the known public domains, such as in the databases of patents in WIPO, USPTO, Espacenet, CNIPA, JPO, etc.

The antigen binding antibodies/proteins of the present invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full-length antibody, a (Fab′)2 fragment, a Fab fragment, or equivalent thereof (such as scFV, bi- tri- or tetra-bodies, Tandabs etc.), when paired with an appropriate light chain. The antibody may be an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. Furthermore, the antigen binding protein may comprise modifications of all classes e.g. IgG dimers, Fc mutants that no longer bind Fc receptors or mediate C1q binding. The antigen binding protein may also be a chimeric antibody of the type described in WO86/001533 which comprises an antigen binding region and a non-immunoglobulin region.

The constant region is selected according to any functionality required e.g. an IgG1 may demonstrate lytic ability through binding to complement and/or will mediate ADCC (antibody dependent cell cytotoxicity).

In one aspect the antigen binding protein is an antibody or antigen binding fragment thereof comprising one or more CDR's according to the invention described herein, or one or both of the heavy or light chain variable domains according to the invention described herein. The antigen binding protein is selected from the group consisting of a dAb, Fab, Fab′, F(ab′)2, Fv, diabody, triabody, tetrabody, miniantibody, and a minibody.

In one aspect of the present invention the antigen binding protein is a humanized or chimeric antibody, in a further aspect the antibody is humanized.

In another aspect, the antibody is a monoclonal antibody or a bispecific antibody.

In another aspect the antigen binding protein binds to human antigens with high affinity for example when measured by Biacore or ForteBio, the antigen binding protein binds to human antigens with an affinity of 20 nM or less or an affinity of 15 nM or less or an affinity of 5 nM or less or an affinity of 1000 pM or less or an affinity of 500 pM or less or an affinity of 400 pM or less, or 300 pM or less or for example about 120 pM. In a further embodiment the antigen binding protein binds to human antigens when measured by Biacore of between about 100 pM and about 500 pM or between about 100 pM and about 400 pM, or between about 100 pM and about 300 pM. In one embodiment of the present invention the antigen binding protein binds antigens with an affinity of less than 150 pM.

In one such embodiment, this is measured by Biacore or ForteBio.

In another aspect the antigen binding protein/antibody binds to human antigens in a cell neutralisation assay wherein the antigen binding protein has an IC₅₀ of between about 1 nM and about 500 nM, or between about 1 nM and about 100 nM, or between about 1 nM and about 50 nM, or between about 1 nM and about 25 nM, or between about 5 nM and about 15 nM. In a further embodiment of the present invention the antigen binding protein binds antigens and neutralizes antigens in a cell neutralization assay wherein the antigen binding protein has an IC₅₀ of about 10 nM.

The antigen binding proteins, for example antibodies of the present invention may be produced by transfection of a host cell with an expression vector comprising the coding sequence for the antigen binding protein of the invention. An expression vector or recombinant plasmid is produced by placing these coding sequences for the antigen binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies. Similarly, a second expression vector can be produced having a DNA sequence which encodes a complementary antigen binding protein light or heavy chain. In certain embodiments this second expression vector is identical to the first except insofar as the coding sequences and selectable markers are concerned, so to ensure as far as possible that each polypeptide chain is functionally expressed. Alternatively, the heavy and light chain coding sequences for the antigen binding protein may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antigen binding protein of the invention. The antigen binding protein which includes the association of both the recombinant heavy chain and/or light chain is screened from culture by appropriate assay, such as ELISA or RIA. Similar conventional techniques may be employed to construct other antigen binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham Bioscience (Buckinghamshire, United Kingdom) or GenScript (Nanjing, China). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen binding proteins of the present invention. Host cells useful for the cloning and other manipulations of these cloning vectors are also conventional. However, cells from various strains of E. Coli may be used for replication of the cloning vectors and other steps in the construction of antigen binding proteins of this invention.

Suitable host cells or cell lines for the expression of the antigen binding proteins of the invention include mammalian cells such as NS0, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns.

Alternatively, other eukaryotic cell lines may be employed. The selection of suitable mammalian host cells and methods for transformation, culture, amplification, screening and product production and purification are known in the art. See, e.g., Sambrook et al., (1989). Molecular cloning: a laboratory manual, 2nd ed. Cold Spring Harbor Laboratory, Cold Spring Harbor, N.Y.

Bacterial cells may prove useful as host cells suitable for the expression of the recombinant Fabs or other embodiments of the present invention (see, e.g., Pluckthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. Coli used for expression are well-known as host cells in the field of biotechnology. Various strains of B. Subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and McGuire, S. et al, Trends Genet. (2004) 20, 384-391 and references cited therein.

The general methods by which the vectors may be constructed, the transfection methods required to produce the host cells of the invention, and culture methods necessary to produce the antigen binding protein of the invention from such host cell may all be conventional techniques. Typically, the culture method of the present invention is a serum-free culture method, usually by culturing cells serum-free in suspension. Likewise, once produced, the antigen binding proteins of the invention may be purified from the cell culture contents according to standard procedures of the art, including ammonium precipitation, affinity columns, column chromatography, gel electrophoresis and the like. Such techniques are within the skill of the art and do not limit this invention. For example, preparations of altered antibodies are described in WO 99/058679 and WO 96/016990. Yet another method of expression of the antigen binding proteins may utilize expression in a transgenic animal, such as described in U.S. Pat. No. 4,873,316. This relates to an expression system using the animals casein promoter which when transgenically incorporated into a mammal permits the female to produce the desired recombinant protein in its milk.

In a further embodiment of the invention there is provided a method of producing an antibody of the invention which method comprises the step of culturing a host cell transformed or transfected with a vector encoding the light and/or heavy chain of the antibody of the invention and recovering the antibody thereby produced.

In accordance with the present invention there is provided a method of producing an antibody of the present invention which binds to and neutralises the activity of human ANTIGENS which method comprises the steps of; providing a first vector encoding a heavy chain of the antibody; providing a second vector encoding a light chain of the antibody; transforming a mammalian host cell (e.g. CHO) with said first and second vectors; culturing the host cell of step (c) under conditions conducive to the secretion of the antibody from said host cell into said culture media; recovering the secreted antibody of step (d).

Once expressed by the desired method, the antibody is then examined for in vitro activity by use of an appropriate assay. Presently conventional ELISA assay formats are employed to assess qualitative and quantitative binding of the antibody to ANTIGENS. Additionally, other in vitro assays may also be used to verify neutralizing efficacy prior to subsequent human clinical studies performed to evaluate the persistence of the antibody in the body despite the usual clearance mechanisms.

The dose and duration of treatment relates to the relative duration of the molecules (the antibody and the antibody-drug conjugate) of the present invention in the human circulation, and can be adjusted by one of skill in the art depending upon the condition being treated and the general health of the patient. It is envisaged that repeated dosing (e.g. once a week or once every two weeks or once every 3 weeks or once every 4 weeks) over an extended time period (e.g. four to six months) may be required to achieve maximal therapeutic efficacy.

In one embodiment of the present invention there is provided a recombinant transformed, transfected or transduced host cell comprising at least one expression cassette, for example where the expression cassette comprises a polynucleotide encoding a heavy chain of an antigen binding protein according to the invention described herein and further comprises a polynucleotide encoding a light chain of an antigen binding protein according to the invention described herein or where there are two expression cassettes and the 1.sup.st encodes the light chain and the second encodes the heavy chain. For example in one embodiment the first expression cassette comprises a polynucleotide encoding a heavy chain of an antigen binding protein comprising a constant region or antigen binding fragment thereof which is linked to a constant region according to the invention described herein and further comprises a second cassette comprising a polynucleotide encoding a light chain of an antigen binding protein comprising a constant region or antigen binding fragment thereof which is linked to a constant region according to the invention described herein for example the first expression cassette comprises a polynucleotide encoding a heavy chain and a second expression cassette comprising a polynucleotide encoding a light chain.

In another embodiment of the invention there is provided a stably transformed host cell comprising a vector comprising one or more expression cassettes encoding a heavy chain and/or a light chain of the antibody comprising a constant region or antigen binding fragment thereof which is linked to a constant region as described herein. For example, such host cells may comprise a first vector encoding the light chain and a second vector encoding the heavy chain, for example the first vector encodes a heavy chain and a second vector encoding a light chain.

In another embodiment of the present invention there is provided a host cell according to the invention described herein wherein the cell is eukaryotic, for example where the cell is mammalian. Examples of such cell lines include CHO or NSO.

In another embodiment of the present invention there is provided a method for the production of an antibody comprising a constant region or antigen binding fragment thereof which is linked to a constant region according to the invention described herein which method comprises the step of culturing a host cell in a culture media, for example serum-free culture media.

In another embodiment of the present invention there is provided a method according to the invention described herein wherein said antibody is further purified to at least 95% or greater (e.g. 98% or greater) with respect to said antibody containing serum-free culture media.

In yet another embodiment there is provided a pharmaceutical composition comprising an antigen binding protein and a pharmaceutically acceptable carrier.

In another embodiment of the present invention there is provided a kit-of-parts comprising the composition according to the invention described herein described together with instructions for use.

The mode of administration of the therapeutic agent of the invention may be any suitable route which delivers the agent to the host. The antigen binding proteins, and pharmaceutical compositions of the invention are particularly useful for parenteral administration, i.e., subcutaneously (s.c.), intrathecally, intraperitoneally, intramuscularly (i.m.) or intravenously (i.v.). In one such embodiment the antigen binding proteins of the present invention are administered intravenously or subcutaneously.

Therapeutic agents of the invention may be prepared as pharmaceutical compositions containing an effective amount of the antigen binding protein of the invention as an active ingredient in a pharmaceutically acceptable carrier. In one embodiment the prophylactic agent of the invention is an aqueous suspension or solution containing the antigen binding protein in a form ready for injection. In one embodiment the suspension or solution is buffered at physiological pH. In one embodiment the compositions for parenteral administration will comprise a solution of the antigen binding protein of the invention or a cocktail thereof dissolved in a pharmaceutically acceptable carrier. In one embodiment the carrier is an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen binding protein of the invention in such pharmaceutical formulation can vary widely, i.e., from less than about 0.5%, usually at or at least about 1% to as much as about 15 or 20% by weight and will be selected primarily based on fluid volumes, viscosities, etc., according to the particular mode of administration selected.

Thus, a pharmaceutical composition of the invention for intravenous infusion could be made up to contain about 250 ml of sterile Ringer's solution, and about 1 to about 30 or 5 mg to about 25 mg of an antigen binding protein of the invention per ml of Ringer's solution. Actual methods for preparing parenterally administrable compositions are well known or will be apparent to those skilled in the art and are described in more detail in, for example, Remington's Pharmaceutical Science, 15.sup.th ed., Mack Publishing Company, Easton, PA, USA. For the preparation of intravenously administrable antigen binding protein formulations of the invention see Parkins D. and Lasmar U. “The formulation of Biopharmaceutical products”, Pharm. Sci. Tech. Today, 3 (2000) 129-137; Wang, W “Instability, stabilisation and formulation of liquid protein pharmaceuticals”, Int. J. Pharm 185 (1999) 129-188; Jorgensen, L. et al, “Recent trends in stabilising peptides and proteins in pharmaceutical formulation-considerations in the choice of excipients” Expert Opin Drug Deliv. 6 (2009) 1219-1230; Akers, M. J. “Excipient-Drug interactions in Parenteral Formulations”, J. Pharm Sci 91 (2002) 2283-2300; Imamura, K et al “Effects of types of sugar on stabilization of Protein in the dried state”, J Pharm Sci 92 (2003) 266-274; Izutsu, Kkojima, S. “Excipient crystallinity and its protein-structure-stabilizing effect during freeze-drying”, J. Pharm. Pharmacol, 54 (2002) 1033-1039; Johnson, R, et al “Mannitol-sucrose mixtures—versatile formulations for protein lyophilization”, J. Pharm. Sci, 91 (2002) 914-922; Kerwin B. “Polysorbates 20 and 80 used in the formulation of protein biotherapeutics: structure and degradation pathways” J. Pharm Sci. 97 (2008) 2924-2935; Ha, E., et al “Peroxide formation in polysorbate 80 and protein stability”, J. Pharm Sci, 91 (2002), 2252-2264, and He, F., et al, “Effect of sugar molecules on the viscosity of high concentration monoclonal antibody solutions” Pharm Res. 28 (2011) 1552-1560; and the entire contents of which are incorporated herein by reference and to which the reader is specifically referred.

In one embodiment the antibody of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of about 0.1 to about 200 mg/kg, for example about 1 to about 20 mg/kg, for example about 10 to about 20 mg/kg or for example about 1 to about 15 mg/kg, for example about 5 to about 15 mg/kg. To effectively treat conditions such as Multiple myeloma, SLE or IPT in a human, suitable doses may be within the range of about 0.1 to about 2000 mg, for example about 0.1 to about 500 mg, for example about 500 mg, for example about 0.1 to about 150 mg, or about 0.1 to about 80 mg, or about 0.1 to about 60 mg, or about 0.1 to about 40 mg, or for example about 1 to about 100 mg, or about 1 to about 50 mg, of an antigen binding protein of this invention, which may be administered parenterally, for example subcutaneously, intravenously or intramuscularly. Such dose may, if necessary, be repeated at appropriate time intervals selected as appropriate by a physician.

The antigen binding proteins described herein can be lyophilized for storage and reconstituted in a suitable carrier prior to use. This technique has been shown to be effective with conventional immunoglobulins and art-known peroxidise and reconstitution techniques can be employed.

In another aspect of the invention there is provided an antigen binding protein as herein described for use in a medicament.

In one aspect of the present invention there is provided an antigen binding protein according to the invention as herein described for use in the treatment of rheumatoid arthitis, Type 1 Diabetes Mellitus, multiple sclerosis or psoriasis wherein said method comprises the step of administering to said patient a therapeutically effective amount of the antigen binding protein as described herein.

In one embodiment of the present invention, methods are provided for treating cancer in a human comprising administering to said human an antigen binding protein that specifically binds to antigens on the tumor cells. In some instances, the antigen binding protein is part of an immunoconjugate.

The term “antibody-drug conjugate (ADC),” as used herein, refers to a compound comprising a monoclonal antibody (mAb) attached to a cytotoxic agent (generally a small molecule drug with a high systemic toxicity) via chemical linkers. The ADC of this invention is represented as the formula of:

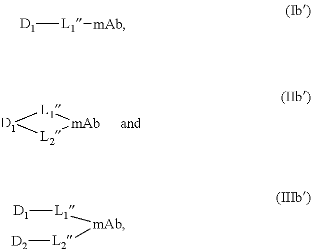

wherein D₁ and D₂ are a small molecule cytotoxin or a functional small molecule, in general called payload; L₁ and L₂ are a function linker that has an affinity ligand; and mAb is a monoclonal antibody. In some embodiments, an ADC may comprise a small molecule cytotoxin that has been chemically modified to contain a linker with an affinity ligand, or a linker containing an affinity ligand is part of payload which is called a traceless linker. The linker is generally used to conjugate the cytotoxin to the antibody, or antigen-binding fragment thereof. Upon binding to the target antigen on the surface of a cell, the ADC is internalized and trafficked to the lysosome where the cytotoxin is released by either proteolysis of a cleavable linker (e.g., by cathepsin B found in the lysosome) or by proteolytic degradation of the antibody, if attached to the cytotoxin via a non-cleavable linker. The cytotoxin then translocates out of the lysosome and into the cytosol or nucleus, where it can then bind to its target, depending on its mechanism of action.

The antibody-drug conjugate described herein may comprise a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

The ADC may comprise an antigen-binding fragment of an antibody. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Kabat E A, Wu T T., J Immunol. 1991, 147(5): 1709-19) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites (see, e.g. Hudson P J, Kortt A A, J Immunol Methods. 1999, 231(1-2): 177-89; Holliger P, Winter G. Cancer Immunol Immunother. 1997, 45(3-4):128-30).

The monoclonal antibody, or an antigen-binding fragment thereof, directed against a certain antigen may comprise any suitable binding affinity to the antigen or an epitope thereof. The term “affinity” refers to the equilibrium constant for the reversible binding of two agents and is expressed as the dissociation constant (K_{D}). The affinity of an antibody or antigen-binding fragment thereof for an antigen or epitope of interest can be measured using any method known in the art. Such methods include, for example, fluorescence activated cell sorting (FACS), surface plasmon resonance (e.g., Biacore™, ProteOn™), biolayer interferometry (BLI, e.g. Octet), kinetics exclusion assay (e.g. KinExA™), separable beads (e.g., magnetic beads), antigen panning, and/or ELISA (see, e.g., J R Crowther, Methods Mol Biol. 2000, 149: III-IV, 1-413). It is known in the art that the binding affinity of a particular antibody will vary depending on the method that is used to analyze the binding affinity.

Affinity of a binding agent to a ligand, such as affinity of an antibody for an epitope, can be, for example, from about 1 picomolar (pM) to about 1 micromolar (1 μM) (e.g., from about 1 picomolar (pM) to about 1 nanomolar (nM), or from about 1 nM to about 1 micromolar (μM)). In one embodiment, the monoclonal antibody or an antigen-binding fragment thereof may bind to a certain antigen with a Kd less than or equal to 100 nanomolar (e.g., 100 nM, about 90 nM, about 80 nM, about 70 nM, about 60 nM, about 50 nM, about 40 nM, about 30 nM, about 20 nM, or about 10 nM, or a range defined by any two of the foregoing values).

In another embodiment, the monoclonal antibody may bind to a certain antigen with a Kd less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

In another embodiment, the monoclonal antibody may bind to A CERTAIN ANTIGEN with a Kd less than or equal to 200 pM (e.g., about 190 pM, about 175 pM, about 150 pM, about 125 pM, about 110 pM, about 100 pM, about 90 pM, about 80 pM, about 70 pM, about 60 pM, about 50 pM, about 40 pM, about 30 pM, about 25 pM, about 20 pM, about 15 pM, about 10 pM, about 5 pM, about 1 pM, or a range defined by any two of the foregoing values).

In one embodiment, the affinity of the antibody or antigen-binding fragment thereof, as measured by surface plasmon resonance (SPR), is about 90 nM, about 80 nM, about 70 nM, about 60 nM, about 50 nM, about 40 nM, about 30 nM, or a range defined by any two of the foregoing values, for example, about 50 nM to about 70 nM, about 55 nM to about 65 nM, or about 58 nM to about 62 nM.

In one embodiment, the affinity of the antibody or antigen-binding fragment thereof to membrane-bound antigens, as measured by FACS, is less than or equal to 10 nanomolar (e.g., about 9 nM, about 8 nM, about 7 nM, about 6 nM, about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM, about 0.1 nM, about 0.05 nM, about 0.02 nM, about 0.01 nM, about 0.001 nM, or a range defined by any two of the foregoing values).

An antigen-binding portion or fragment of a monoclonal antibody can be of any size so long as the portion binds to the antigens. In this respect, an antigen binding portion or fragment of the monoclonal antibody directed against a certain antigen desirably comprises between about 5 and 50 amino acids (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or a range defined by any two of the foregoing values).

In one embodiment, the antibody-drug conjugate comprises a variable region of a monoclonal antibody. In this respect, the ADC may comprise a light chain variable region, a heavy chain variable region, or both a light chain variable region and a heavy chain variable region of a monoclonal antibody.

The monoclonal antibody, or antigen-binding fragment thereof, may be conjugated to a cytotoxin using any suitable method known in the art, including site-specific or non-site specific conjugation methods. Conventional conjugation strategies for antibodies typically rely on randomly (i.e., non-specifically) conjugating the payload to the antibody, antigen-binding fragment thereof, through lysines or cysteines. Accordingly, in some aspects the antibody or antigen-binding fragment thereof is randomly conjugated to a cytotoxic agent, for example, by partial reduction of the antibody or antibody fragment, followed by reaction with a desired agent with or without a linker moiety attached. For example, the antibody or antigen-binding fragment thereof may be reduced using dithiothreitol (DTT), TCEP, thiolethenol or a similar reducing agent. The cytotoxic agent, with or without a linker moiety attached thereto, can then be added at a molar excess to the reduced antibody or antibody fragment in the presence of dimethyl sulfoxide (DMSO), or DMA. After conjugation, excess free cysteine may be added to quench unreacted agent. The cytotoxic agent, with or without a linker moiety having an amino-reactivable, or phenol-reactivable, or the others reactivable group (e.g. NHS, PFP) thereto, can be added directly at a molar excess to the antibody or antibody fragment in the presence of DMSO, or DMA to form a conjugate. The reaction mixture may then be purified through chromatography or buffer-exchanged into a suitable buffer, such as phosphate buffered saline (PBS), citrate buffer, or histidine buffer.

The terms “cytotoxin” and “cytotoxic agent” refer to any molecule that inhibits or prevents the function of cells and/or causes destruction of cells (cell death), and/or exerts anti-proliferative effects. A cytotoxin or cytotoxic agent of an ADC also is referred to in the art as the “payload” of the ADC. A number of classes of cytotoxic agents are known in the art to have potential utility in ADC molecules and can be used in the ADC described herein. Such classes of cytotoxic agents include, for example, anti-microtubule agents (e.g., tubulysins, auristatins and maytansinoids), DNA minor groove binders (e.g. pyrrolobenzodiazepines (PBDs) or indolinobenzodiazepines (IGN) and their dimers), RNA polymerase II inhibitors (e.g., amatoxins), inhibitor of DNA topoisomerase I (e.g., camptothecins) and DNA alkylating agents (e.g., duocarmycin, CC-1065, pyrrolobenzodiazepine dimers or pseudodimers or indolinobenzodiazepine pseudodimers). Examples of specific cytotoxic agents that may be used in the ADC described herein include, but are not limited to, tubulysins, amanitins, auristatins, calicheamicin, camptothecins, daunomycins, doxorubicins, duocarmycins, dolastatins, enediynes, lexitropsins, taxanes, puromycins, maytansinoids, vinca alkaloids, and pyrrolobenzodiazepines (PBDs). More specifically, the cytotoxic agent may be, for example tubulysins, auristatins (AFP, MMAF, MMAE, AEB, AEVB, E), paclitaxels, docetaxels, CC-1065 (duocarmysin, DC1, DC4, CBI-dimers), camptothecins (SN-38, topotecan, exatecan), morpholino-doxorubicin, rhizoxin, cyanomorpholino-doxorubicin, dolastatin-10, echinomycin, combretatstatin, chalicheamicin, maytansine (DM1, DM4, DM21), vinblastine, methotrexate, netropsin, or derivatives or analogs thereof. Cytotoxins suitable for use in ADCs are also described in, for example, International Patent Application Publication No. PCT/CN2021/128453.

In general, a chemotherapeutic agent or a functional compound can also be conjugated to the antibody of this invention. A chemotherapeutic agent or a functional compound is selected from the group consisting of:
a). an alkylating agent: selected from the group consisting of nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: comprising busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: comprising vincristine, vinblastine, vindesine, vinorelbine, and navelbin; Taxoids: comprising paclitaxel, docetaxol and their analogs, Maytansinoids comprising DM1, DM2, DM3, DM4, DM5, DM6, DM7, maytansine, ansamitocins and their analogs, cryptophycins (including the group consisting of cryptophycin 1 and cryptophycin 8); epothilones, eleutherobin, discodermolide, bryostatins, dolostatins, auristatins, tubulysins, cephalostatins; pancratistatin; erbulins, a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (comprising mycophenolic acid, tiazofurin, ribavirin, EICAR); Ribonucleotide reductase Inhibitors: (comprising hydroxyurea, deferoxamine)]; [pyrimidine analogs: Uracil analogs: (comprising ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex)); Cytosine analogs: (comprising cytarabine, cytosine arabinoside, fludarabine); Purine analogs: (comprising azathioprine, fludarabine, mercaptopurine, thiamiprine, thioguanine)]; folic acid replenisher, frolinic acid}; and Inhibitors of nicotinamide phosphoribosyltransferase (NAMPT);
e). A hormonal therapy: selected from the group consisting of {Receptor antagonists: [Anti-estrogen: (comprising megestrol, raloxifene, tamoxifen); LHRH agonists: (comprising goscrclin, leuprolide acetate); Anti-androgens: (comprising bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D₃ analogs: (comprising CB 1093, EB 1089 KH 1060, cholecalciferol, ergocalciferol); Photodynamic therapies: (comprising verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, neratinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group consisting of olaparib, niraparib, iniparib, talazoparib, veliparib, CEP 9722 (Cephalon's), E7016 (Eisai's), BGB-290 (BeiGene's), or 3-aminobenzamide.
h). An antibiotic, selected from the group consisting of an enediyne antibiotic (selected from the group consisting of calicheamicin, calicheamicin y1, 81, al or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (selected from staurosporine), Actinomycins (comprising Actinomycin D, dactinomycin), amanitins, Bleomycins (comprising bleomycin A2, bleomycin B2, peplomycin), Anthracyclines (comprising daunorubicin, doxorubicin (adriamycin), idarubicin, epirubicin, pirarubicin, zorubicin, mtoxantrone, MDR inhibitors or verapamil, Ca2+ ATPase inhibitors or thapsigargin, Histone deacetylase inhibitors ((comprising Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group consisting of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes (including the group consisting of T-2 toxin, verrucarin A, roridin A and anguidine); urethane, siRNA, antisense drugs;

(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem/cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef);
f). Glycopeptides: bleomycin, vancomycin (oritavancin, telavancin), teicoplanin (dalbavancin), ramoplanin;
g). Glycylcyclines: tigecycline;
h). β-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:

a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of 8-1-thymidine and 8-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.

(5). A radioisotope that can be selected from the group consisting of (radionuclides) ³H, ¹¹C, ¹⁴C, ¹⁸F, ³²p, ³⁵S, ⁶⁴Cu, ⁶⁸Ga, ⁸⁶Y, ⁹⁰Y, ⁹⁹Tc, ¹¹¹In, ¹²³I, ¹²⁴I, ¹²⁵I, ¹³¹I, ¹³³Xe, ¹⁷⁷Lu, ²⁰³Pb, ²¹²Pb, ²¹¹At, ²¹³Bi, ²²⁴Ra and ²²⁵Ac. The metal radionuclides, ⁶⁴Cu, ⁶⁸Ga, ⁸⁶Y, ⁹⁰Y, ⁹⁹Tc, ¹¹¹In, ¹³³Xe, ¹⁷⁷Lu, ²⁰³Pb, ²¹²Pb, ²¹¹At, ²¹³Bi, ²²⁴Ra and ²²⁵Ac linked to an antibody through a chelator and a linker L₁ of this patent application. The chelator is selected from DOTA, TETA, NOTA, NETA, DTPA, HBED, SHBED, and their structures are illustrated below:

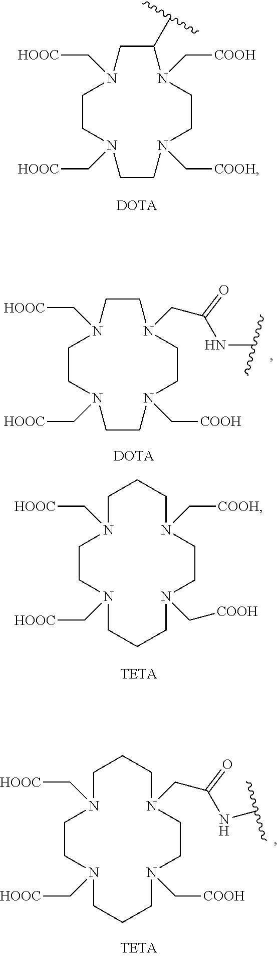

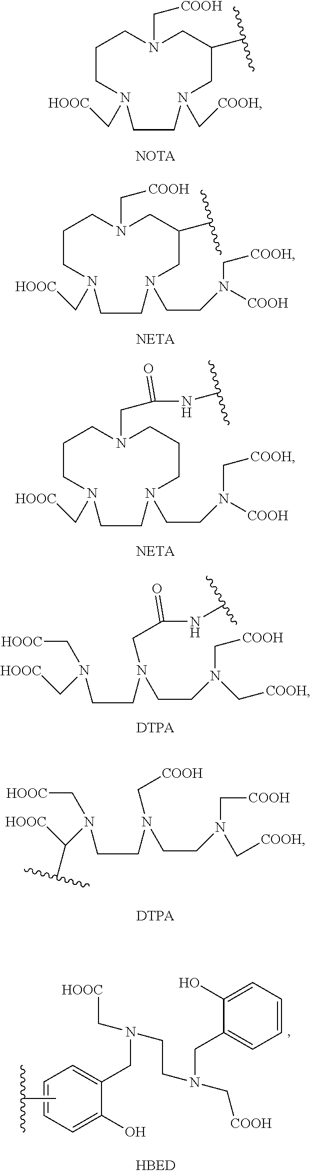

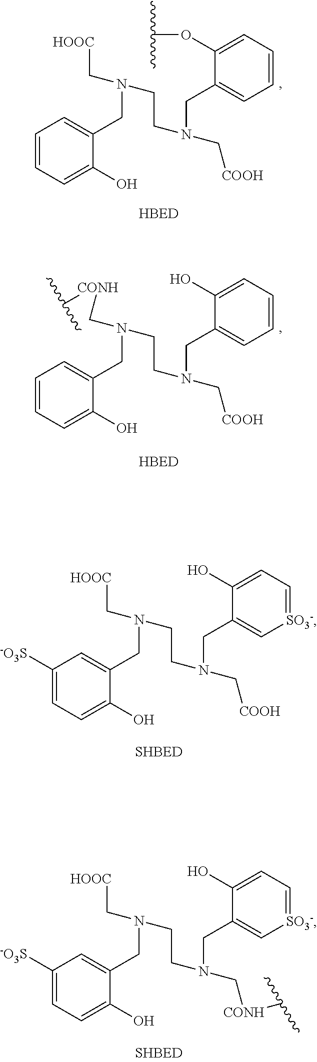

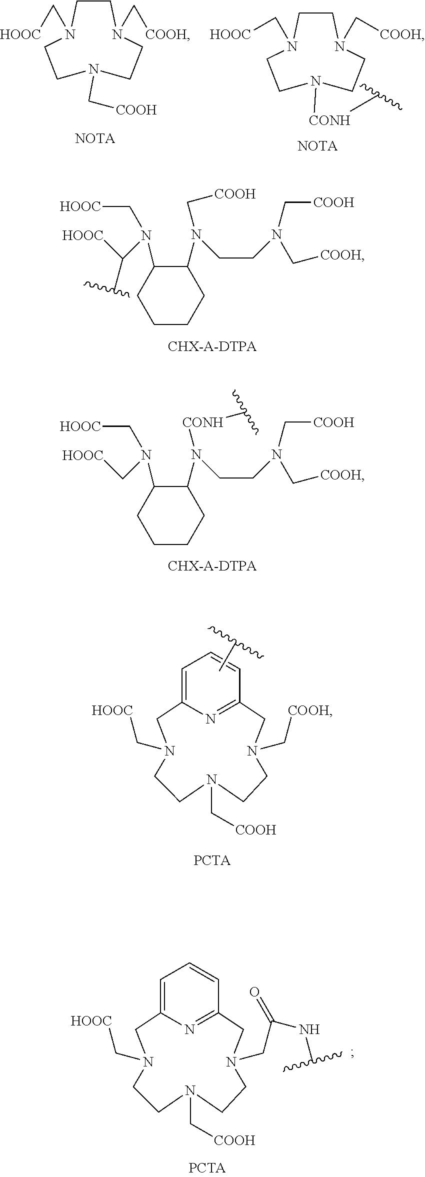

(6). A chromophore molecule, which is capable of absorbing UV light, florescent light, IR light, near IR light, visual light; A class or subclass of xanthophores, erythrophores, iridophores, leucophores, melanophores, cyanophores, fluorophore molecules which are fluorescent chemical compounds reemitting light upon light, visual phototransduction molecules, photophore molecules, luminescence molecules, luciferin compounds; Non-protein organic fluorophores, selected from: Xanthene derivatives (comprising fluorescein, rhodamine, Oregon green, eosin, and Texas red); Cyanine derivatives: (comprising cyanine, indocarbocyanine, oxacarbocyanine, thiacarbocyanine, and merocyanine); Squaraine derivatives and ring-substituted squaraines, including Seta, SeTau, and Square dyes; Naphthalene derivatives (comprising dansyl and prodan derivatives); Coumarin derivatives; Oxadiazole derivatives (comprising pyridyloxazole, nitrobenzoxadiazole and benzoxadiazole); Anthracene derivatives (comprising anthraquinones, including DRAQ5, DRAQ7 and CyTRAK Orange); Pyrene derivatives (cascade blue); Oxazine derivatives (comprising Nile red, Nile blue, cresyl violet, oxazine 170). Acridine derivatives (comprising proflavin, acridine orange, acridine yellow). Arylmethine derivatives (comprising auramine, crystal violet, malachite green). Tetrapyrrole derivatives (comprising porphin, phthalocyanine, bilirubin); Any analogs and derivatives of the following fluorophore compounds comprising CF dye, DRAQ and CyTRAK probes, BODIPY, Alexa Fluor, DyLight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes (APC, RPEPerCP, Phycobilisomes), APC, APCXL, RPE, BPE, Allophycocyanin (APC), Aminocoumarin, APC-Cy7 conjugates, BODIPY-FL, Cascade Blue, Cy2, Cy3, Cy3.5, Cy3B, Cy5, Cy5.5, Cy7, Fluorescein, FluorX, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Seta-555-Azide, Seta-555-DBCO, Seta-555-NHS, Seta-580-NHS, Seta-680-NHS, Seta-780-NHS, Seta-APC-780, Seta-PerCP-680, Seta-R-PE-670, SeTau-380-NHS, SeTau-405-Maleimide, SeTau-405-NHS, SeTau-425-NHS, SeTau-647-NHS, Texas Red, TRITC, TruRed, X-Rhodamine, 7-AAD (7-aminoactinomycin D, CG-selective), Acridine Orange, Chromomycin A3, CyTRAK Orange (red excitation dark), DAPI, DRAQ5, DRAQ7, Ethidium Bromide, Hoechst33258, Hoechst33342, LDS 751, Mithramycin, PropidiumIodide (PI), SYTOX Blue, SYTOX Green, SYTOX Orange, Thiazole Orange, TO-PRO: Cyanine Monomer, TOTO-1, TO-PRO-1, TOTO-3, TO-PRO-3, YOSeta-1, YOYO-1; A fluorophore compound: comprising DCFH (2′7′Dichorodihydro-fluorescein, oxidized form), DHR (Dihydrorhodamine 123, oxidized form, light catalyzes oxidation), Fluo-3 (AM ester. pH>6), Fluo-4 (AM ester. pH 7.2), Indo-1 (AM ester, low/high calcium (Ca2+)), SNARF (pH 6/9), Allophycocyanin (APC), AmCyan1 (tetramer, Clontech), AsRed2 (tetramer, Clontech), Azami Green (monomer), Azurite, B-phycoerythrin (BPE), Cerulean, CyPet, DsRed monomer (Clontech), DsRed2 (“RFP”), EBFP, EBFP2, ECFP, EGFP (weak dimer), Emerald (weak dimer), EYFP (weak dimer), GFP (S65A mutation), GFP (S65C mutation), GFP (S65L mutation), GFP (S65T mutation), GFP (Y66F mutation), GFP (Y66H mutation), GFP (Y66W mutation), GFPuv, HcRed1, J-Red, Katusha, Kusabira Orange (monomer, MBL), mCFP, mCherry, mCitrine, Midoriishi Cyan (dimer, MBL), mKate (TagFP635, monomer), mKeima-Red (monomer), mKO, mOrange, mPlum, mRaspberry, mRFP1 (monomer), mStrawberry, mTFP1, mTurquoise2, P3 (phycobilisome complex), Peridinin Chlorophyll (PerCP), R-phycoerythrin (RPE), T-Sapphire, TagCFP (dimer), TagGFP (dimer), TagRFP (dimer), TagYFP (dimer), tdTomato (tandem dimer), Topaz, TurboFP602 (dimer), TurboFP635 (dimer), TurboGFP (dimer), TurboRFP (dimer), TurboYFP (dimer), Venus, Wild Type GFP, YPet, ZsGreen1 (tetramer), ZsYellow1 (tetramer) and their derivatives.

(7). The cell-binding ligands or receptor agonists, which can be selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, selected from the group consisting of buserelin (Pyr-His-Trp-Ser-Tyr-D-Ser (OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser (OtBu)-Leu-Arg-Pro-AzGly-NH₂), Histrelin (Pyr-His-Trp-Ser-Tyr-D-His (N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl) Ala-Ser-(N-Me)Tyr-D-Asn-Leu-isopropylLys-Pro-D-Ala-NH₂), Cetrorelix (Ac-D-2Nal-D-4-chloroPhe-D-3-(3-pyridyl) Ala-Ser-Tyr-D-Cit-Leu-Arg-Pro-D-Ala-NH₂), Degarelix (Ac-D-2Nal-D-4-chloroPhe-D-3-(3-pyridyl) Ala-Ser-4-aminoPhe (L-hydroorotyl)-D-4-aminoPhe (carba-moyl)-Leu-isopropylLys-Pro-D-Ala-NH₂), and Ganirelix (Ac-D-2Nal-D-4-chloroPhe-D-3-(3-pyridyl) Ala-Ser-Tyr-D-(N9,N10-diethyl)-homoArg-Leu-(N9,N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of αᵥβ₁, αᵥβ₃, αᵥβ₅, αᵥβ₆, α₆β₄, α₇β₁, α_{L}μ₂, α_{IIb}β₃) agonists (selected from the group consisting of GRGDSPK, cyclo (RGDfV) (L1) and its derives [cyclo(—N (Me)R-GDfV), cyclo(R-Sar-DfV), cyclo(RG-N(Me)D-fV), cyclo(RGD-N(Me)f-V), cyclo(RGDf-N (Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors and VEGF receptors' agonists; an immunotherapeutical short antibody-like protein, siRNA or DNA molecule.

(8). The pharmaceutically acceptable salts, acids, derivatives, hydrate or hydrated salt; or a crystalline structure; or an optical isomer, racemate, diastereomer or enantiomer of any of the above drugs.

In another embodiment, the drug D can be polyalkylene glycols that are used for extending the half-life of the cell-binding antibody, or antibody molecule when administered to a mammal. Polyalkylene glycols include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxy PEG-monoaldehydes, hydroxy PEG-monoamines, hydroxyPEG-monohydrazides, hydroxy PEG-monocarbazates, hydroxyl PEG-monoiodoacetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones).

In certain such embodiments, the polyalkylene glycol has a molecular weight of from about 10 Daltons to about 200 kDa, preferably about 88 Da to about 40 kDa; two branches each with a molecular weight of about 88 Da to about 40 kDa; and more preferably two branches, each of about 88 Da to about 20 kDa. In one particular embodiment, the polyalkylene glycol is poly(ethylene)glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095.

In yet another embodiment, D is more preferably a potent cytotoxic agent, selected from a tubulysin and its analogs, a maytansinoid and its analogs, a taxanoid (taxane) and its analogs, a CC-1065 and its analogs, a daunorubicin or doxorubicin and its analogs, an amatoxin and its analogs, a benzodiazepine dimer (e.g., dimers of pyrrolobenzodiazepine (PBD), tomaymycin, anthramycin, indolinobenzodiazepines, imidazobenzothiadiazepines, or oxazolidinobenzo-diazepines) and their analogs, a calicheamicin and the enediyne antibiotic and their analogs, an actinomycin and its analogs, an azaserine and its analogs, a bleomycin and its analogs, an epirubicin and its analogs, a tamoxifen and its analogs, an idarubicin and its analogs, a dolastatin and its analogs, an auristatin (including monomethyl auristatin E (MMAE), MMAF, auristatin PYE, auristatin TP, Auristatins 2-AQ, 6-AQ, EB (AEB), and EFP (AEFP)) and its analogs, a combretastatin, a duocarmycin and its analogs, a camptothecin, a geldanamycin and its analogs, a methotrexate and its analogs, a thiotepa and its analogs, a vindesine and its analogs, a vincristine and its analogs, a hemiasterlin and its analogs, a nazumamide and its analogs, a spliceostatin, a pladienolide, a microginin and its analogs, a radiosumin and its analogs, an alterobactin and its analogs, a microsclerodermin and its analogs, a theonellamide and its analogs, an esperamicin and its analogs, PNU-159682 and its analogs, a protein kinase inhibitor, a MEK inhibitor, a KSP inhibitor, a nicotinamide phosphoribosyltransferase (NAMPT) inhibitor, an immunotoxin, a cell receptor agonist, a cell stimulating molecule or intracellular signaling molecule, one, two or more DNA, RNA, mRNA, small interfering RNA (siRNA), microRNA (miRNA), and PIWI interacting RNAs (piRNA), and stereoisomers, isosteres, analogs, or derivatives thereof.

In certain such embodiments, tubulysin and its analogs are preferably the cytotoxic agent of the present invention. Tubulysins can be isolated from natural sources according to known methods or prepared synthetically according to known methods (e.g. Balasubramanian, R., et al. J. Med. Chem., 2009, 52, 238-40; Wipf, P., et al. Org. Lett., 2004, 6, 4057-60; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Friestad, G. K., Org. Lett., 2004, 6, 3249-52; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49:4809-12; Chai, Y., et al., Chem Biol, 2010, 17:296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; Patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, U.S. application No. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. patent application No. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., U.S. application No. 20110027274. The preferred structures of tubulysins for conjugation of cell binding molecules through process of the present patent application are described in the patent application of PCT/IB2012/053554.

Tubulysin analog having the following formula (IV′):

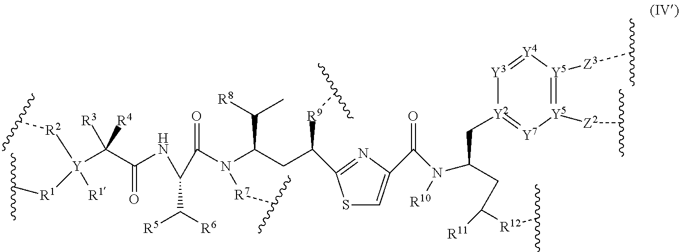

or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently; when two of “ [symbol] ” link to both L₁ and L₂, R¹ and R², or Z² and Z³ are preferably the dual linkage sites;
wherein R¹, R^{1′}, R², R³, and R⁴ are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹R², R¹R³, R²R³, R³R⁴, or R⁵R⁶ form a 3˜7 membered carbocyclic, cycloalkyl, heterocyclic, heterocycloalkyl, aromatic or heteroaromatic ring system; R¹ and R² can be independently absent when they link to L₁ or L₂ independently or simultaneously, Y1 is N or CH;
Preferably R¹, R^{1′}, R², R³, and R⁴ are independently H, C₁˜C₈ alkyl or heteroalkyl and Y1 is N;
wherein R⁵, R⁶, R⁸, R¹⁰ and R¹¹ are independently H, or C₁˜C₄ alkyl or heteroalkyl;
wherein R⁷ is independently H, R¹⁴, —R¹⁴C(═O) X₁R¹⁵; or —R¹⁴X₁R¹⁵; X₁ is O, S, S—S, NH, CH₂ or NR¹⁴.
wherein R⁹ is selected from H, OH, ═O, —OR₁₄, —OC(═O)R¹⁴, —OC(═O)NHR¹⁴, or —OC(═O) NR¹⁴R¹⁵, when R⁹ links L₁ or L₂, R⁹ is-O—, —OC(—O)NH— or —OC(═O)N(R¹⁴)—;
wherein R¹¹ is H, or C₁˜C₈ alkyl or C₃˜C₈ Ar-alkyl;
wherein R¹² is-COOH, —COSH, —CONH₂, CONHNH₂, CONHNHR¹⁵, —CONH(R¹⁵), —COOR¹⁵, —R¹⁵COR₁₆, —R¹⁵COOR₁₆, —R¹⁵C(O)NH₂, —R¹⁵C(O)NHR¹⁶, —COSR¹⁵, R¹⁵S(═O)₂R¹⁶, —R¹⁵P(═O)(OR₁₇)₂, —R¹⁵OP(═O)(OR₁₇)₂, —COOCH₂OP(═O)(OR₁₇)₂, —COX²SO₂R¹⁷, —COOR₁₅X₂R¹⁶ tetrazole, imidazole, or triazole, where X₂ is-O—, —S—, —NH—, —N(R¹⁵)—, —O—R¹⁵—, —S—R¹⁵—, CH₂ or —NHR¹⁵—; when R¹² links L₁ or L₂, R¹² is-C(O)O—, —C(O)NH—, —C(═O)NHS(O)₂R¹⁵—or —C(═O)N(R¹⁵)—;
Preferably R¹² is is-COOH, —CONH₂, CONHNH₂, CONHNHR¹⁵, —CONH(R¹⁵), or —COOR₁₅;
R¹³ and R¹⁴ are independently C₁˜C₈ alkyl, heteroalkyl; C₂-C₈ of alkenyl, alkynyl, heteroalkyl, heterocycloalkyl; C₃-C₈ of aryl, Ar-alkyl; Preferably R¹³ and R¹⁴ are independently C₁˜C₈ alkyl;
Z² and Z³ are independently H, O, S, NH, N(R¹⁵), NHNH, —OH, —SH, —NH₂, NHNH₂, —NH(R¹⁵), —OR₁₅, CO, —COX², —COX²R¹⁶, R¹⁷, F, Cl, Br, I, SR¹⁶, NR¹⁶R¹⁷, N═NR¹⁶, N═R¹⁶, NO₂, SOR 16R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷) OP(O)(OR¹⁷)₂, OC(O)NHR¹⁷; —O—(C₄-C₁₂ glycoside), —N—(C₄-C₁₂ glycoside); C₁˜C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)_{1˜8}, or CO(Aa)_{1˜8} (which are respectively N-terminal or C-terminal 1-8 the same or different amino acids)), or polyethyleneoxy unit of formula (OCH₂CH₂)ₚ or (OCH₂CH(CH₃))ₚ, wherein p is an integer from 0 to about 100, or combination of above groups thereof; X₂ is O, S, S—S, NH, CH₂, OH, SH, NH₂, CHR 15 or NR 15;
Preferably Z² and Z³ are independently H, O, NH, NHNH, —OH, —NH₂, NHNH₂, —NH(R¹⁵), —OR₁₅, CO, —COX², —COX²R¹⁶,
R¹⁵, R¹⁶ and R¹⁷ are independently H, C₁˜C₈ alkyl, heteroalkyl; C₂-C₈ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, Li⁺, Ca²⁺, Mg⁺, Zn²⁺, N⁺(R¹)(R²)(R³)(R⁴), HN⁺(C₂H₅OH)₃ salt; Preferably R¹⁵, R¹⁶ and R¹⁷ are independently H, C₁˜C₈ alkyl;
Y¹ and Y² are independently N or CH; q is 0 or 1; when q=0, Y3 does not exist, Y4, Y5, Y6 and Y7 are independently CH, N, NH, O, S, or N(R¹), thus Y₂, Y4, Y5, Y6 and Y7 form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine;
Examples of the structures of the tubulysin analogs are shown below:

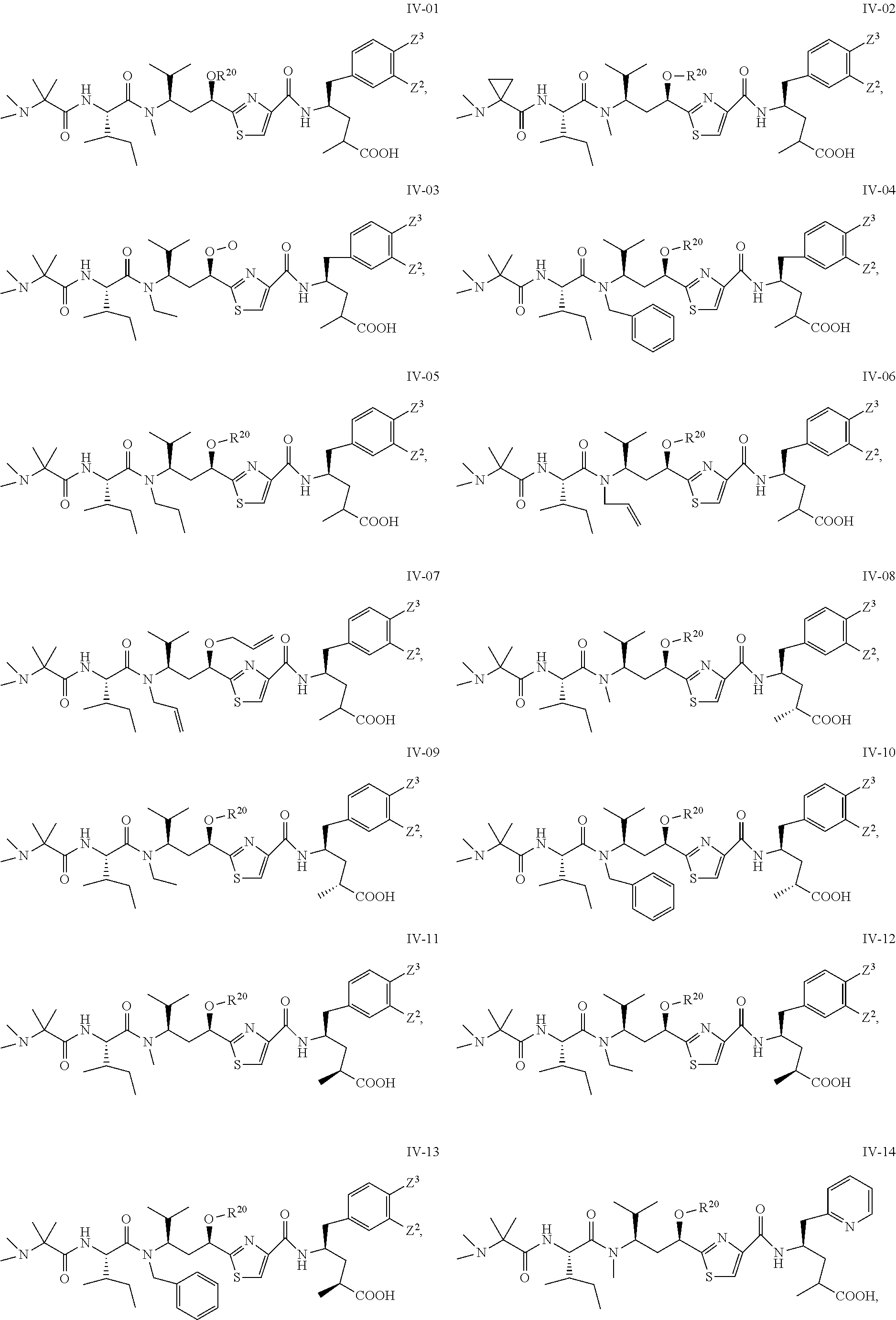

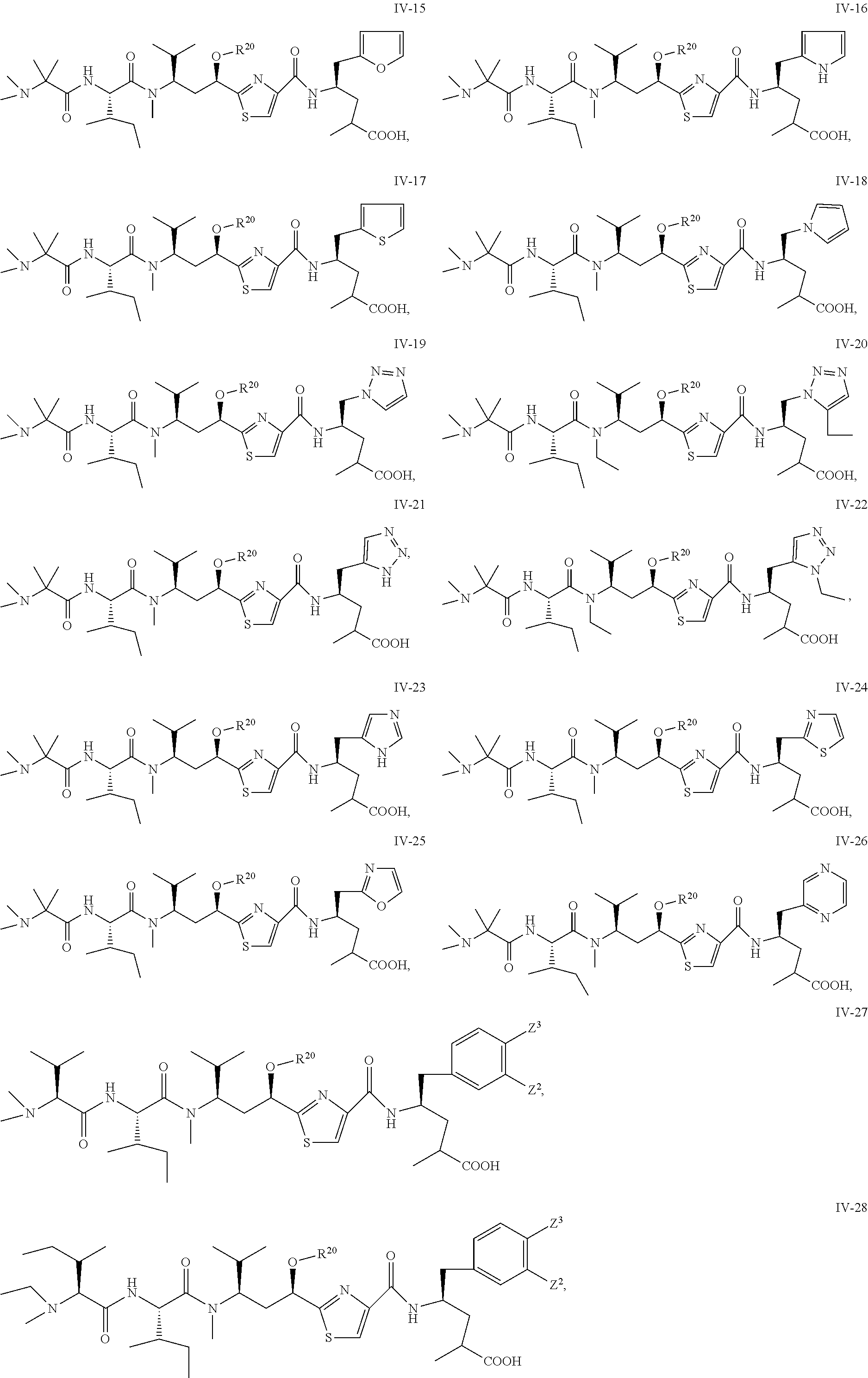

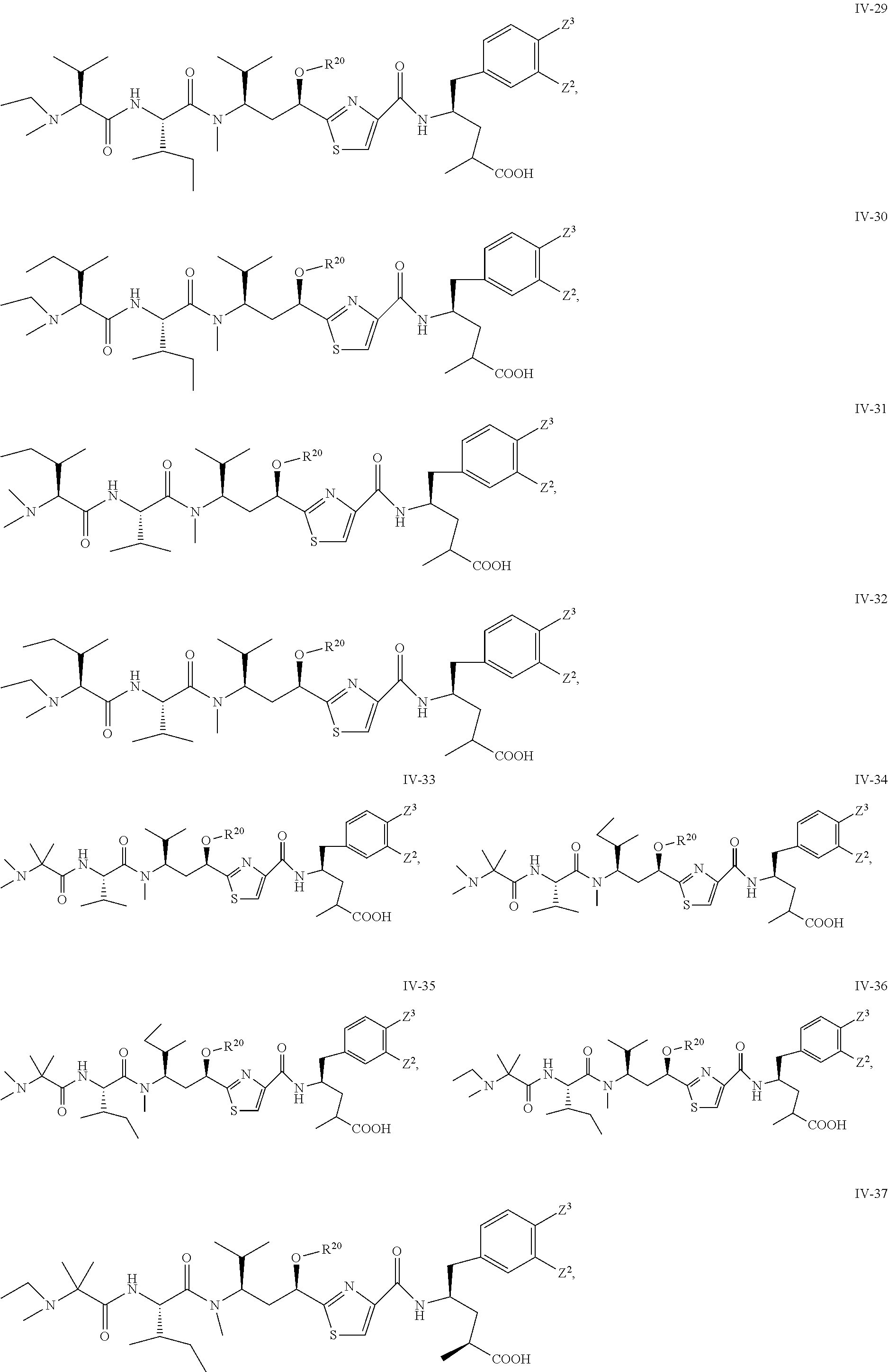

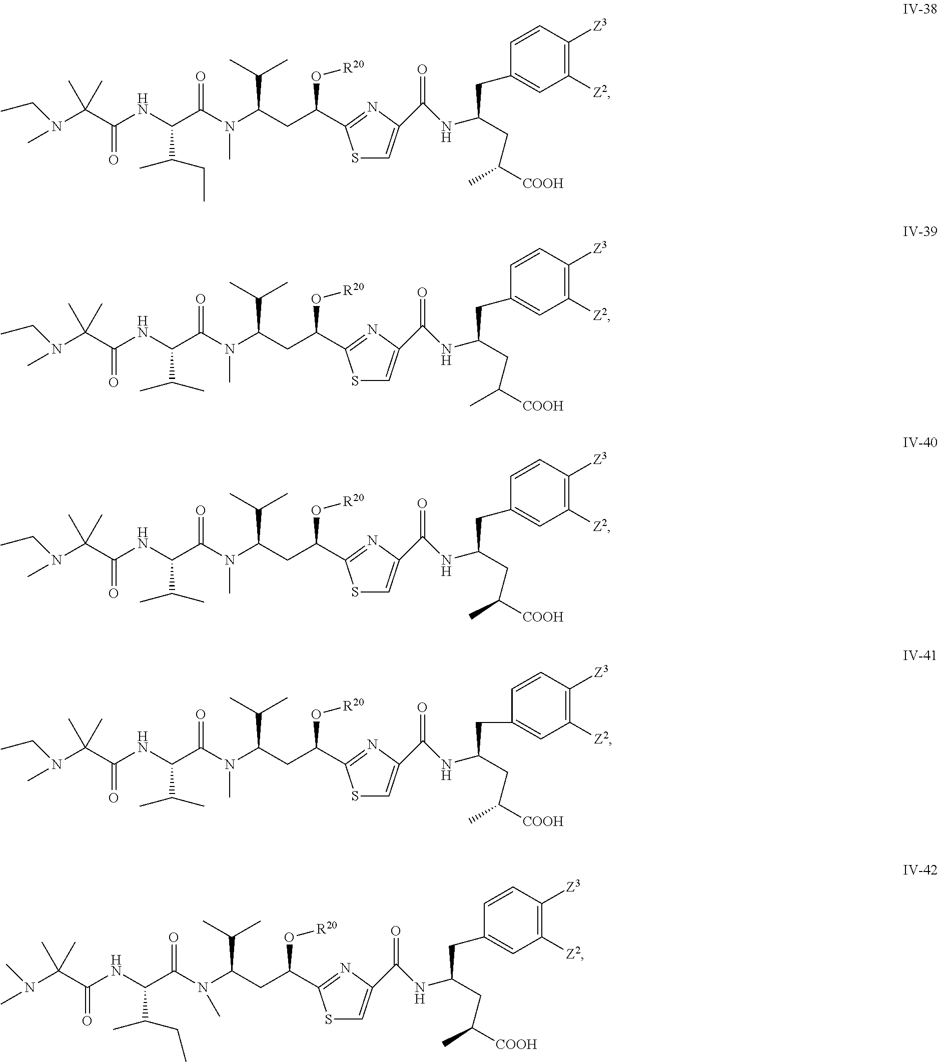

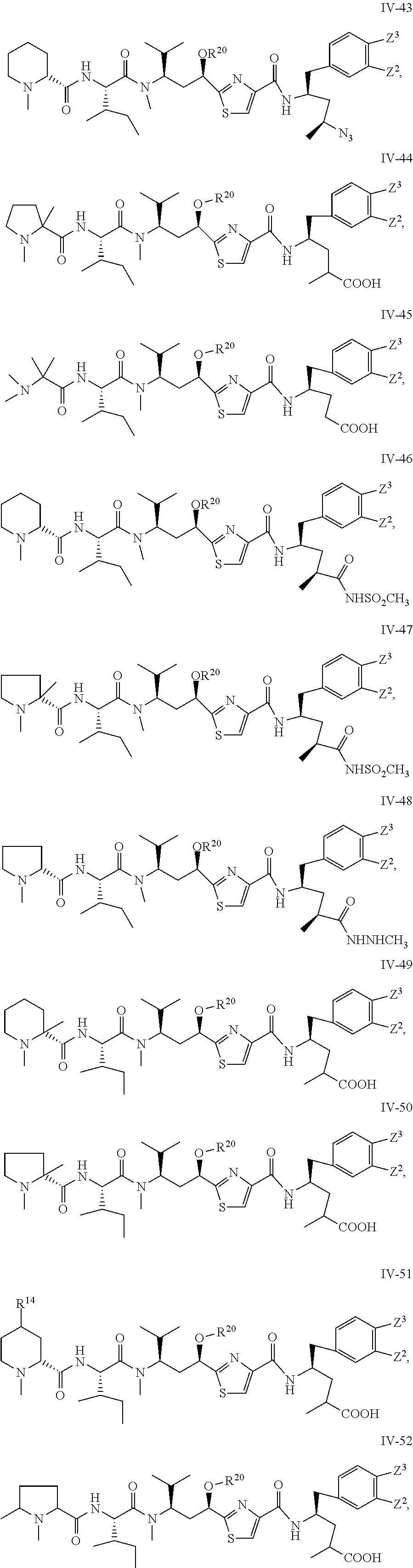

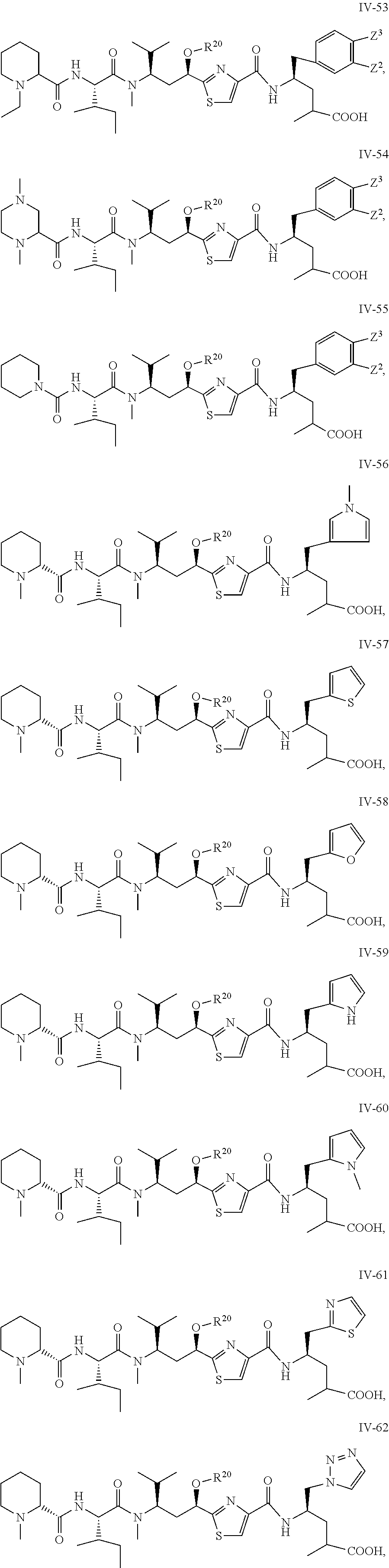

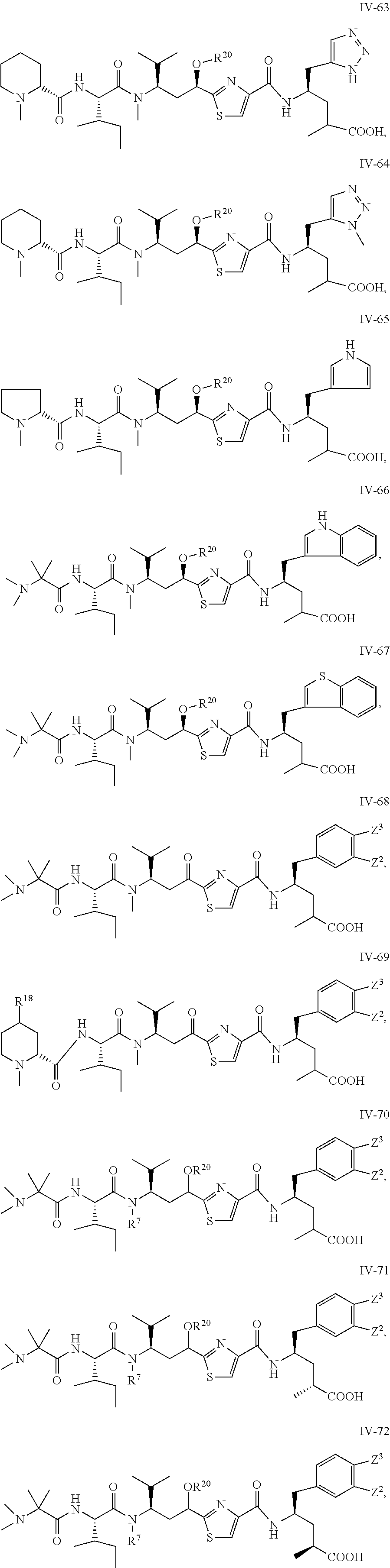

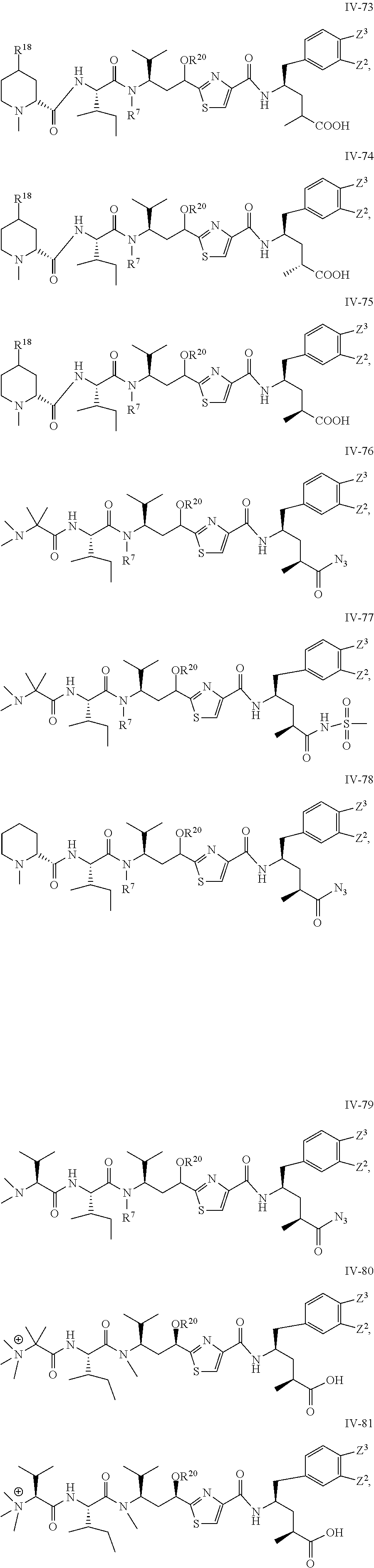

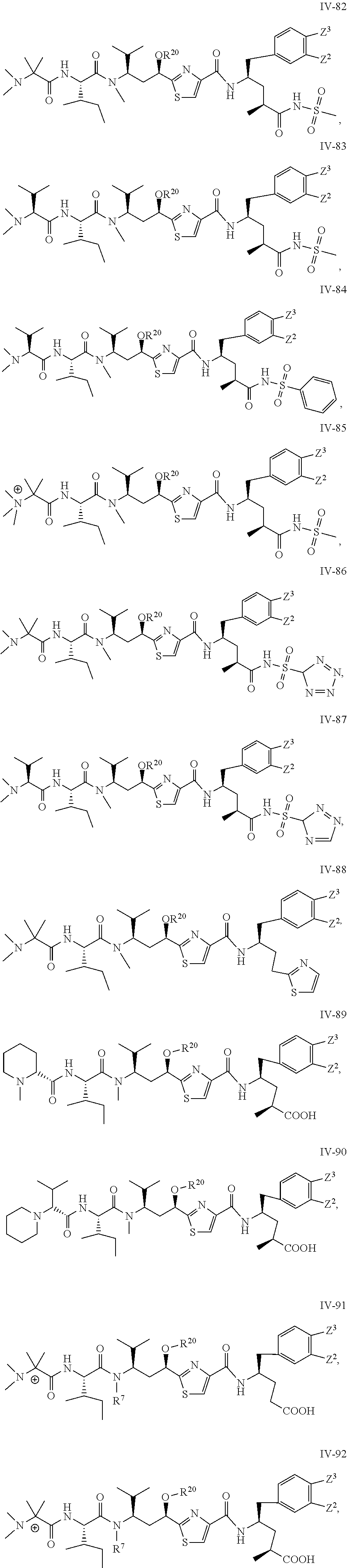

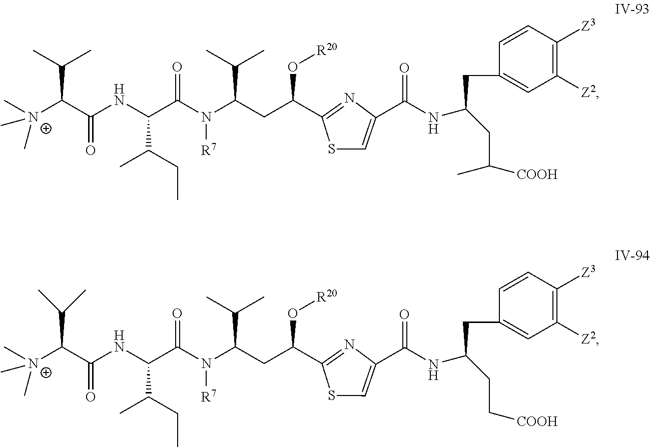

or an isotope of one or more chemical elements, or pharmaceutically acceptable salts, hydrates, or hydrated salts; or the polymorphic crystalline structures of these compounds; or the optical isomers, diastereomers or enantiomers;
wherein R²⁰ is H; C₁-C₈ of linear or branched alkyl or heteroalkyl; C₂-C₈ of alkylcarbonyl, carbonate (—C(O)OR₁₇), carbamate (—C(O)NR¹⁷R¹⁸); or 1-8 carbon atoms of carboxylate, esters, ether, or amide; or 1˜8 amino acids; or polyethyleneoxy unit of formula (OCH₂CH₂)ₚ or (OCH₂CH(CH₃))ₚ, wherein p is an integer from 0 to about 100; or R²⁰ is absent and the oxygen forms a ketone, or combination above groups thereof;
Z³ and Z³ are independently H, OH, NH₂, O, NH, COOH, COO, C(O), C(O)NH, C(O)NH₂, R¹⁸, OCH₂OP(O)(OR¹⁸)₂, OC(O)OP(O)(OR¹⁸)₂, OPO(OR₁₈)₂, NHPO(OR₁₈)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), carbonate (—C(O)OR¹⁷), carbamate (—C(O)NR¹⁷R¹⁸); R¹⁷ and R¹⁸ are independently H, linear or branched alkyl or heteroalkyl;
R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁˜C₈ alkyl or carboylate; or pharmaceutical salts;
X is O, S, NH, NHNH, or CH₂;
R⁷ is defined the same above; wherein the linkage sites, “ [symbol] ” in formula IV-01˜IV-79 are having the same indication according to formula (IV′).

In certain such embodiments, calicheamicins and their related enediyne antibiotics are preferably the cytotoxic agent of the present invention. They were described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. Exemplary enediynes include, but are not limited to, calicheamicin, esperamicin, uncialamicin, dynemicin, and their derivatives. The structure of calicheamicins is preferred the following formula:

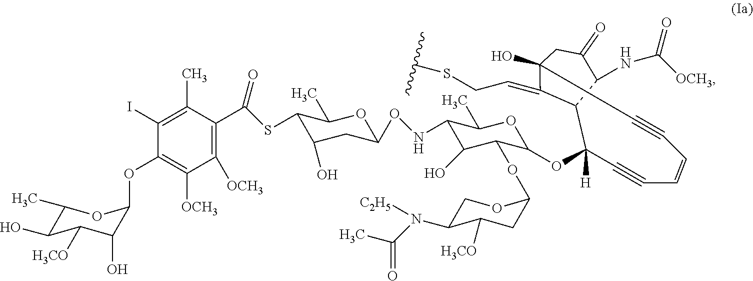 [SMILES: *SC/C=C1\[C@@H]2C(NC(=O)OC)C(=O)C[C@]1(O)C#C/C=C\C#C[C@H]2OC1OC(C)C(NOC2CC(O)C(SC(=O)c3c(C)c(I)c(OC4OC(C)C(O)C(OC)C4O)c(OC)c3OC)C(C)O2)C(O)C1OC1CC(OC)C(N(CC)C(C)=O)CO1]

or an isotope of a chemical element, or a pharmaceutically acceptable salt, hydrates, or hydrated salt; or a polymorphic crystalline structure; or an optical isomer, racemate, diastereomer or enantiomer thereof,
wherein [symbol] is the site linked to L₁ or L₂;
In certain such embodiments, Geldanamycins are benzoquinone ansamycin antibiotic that bind to Hsp90 (Heat Shock Protein 90) and are preferably the cytotoxic agent of the present invention. Exemplary geldanamycins include, but are not limited to, 17-AAG (17-N-Allylamino-17-Demethoxygeldanamycin) and 17-DMAG (17-Dimethylamino-ethylamino-17-demethoxygeldanamycin), having the following formula:

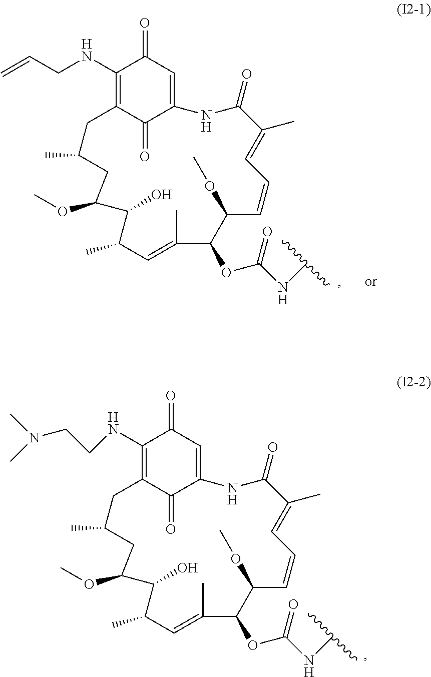 [SMILES: *NC(=O)O[C@H]1/C(C)=C/[C@H](C)[C@@H](O)[C@@H](OC)C[C@H](C)CC2=C(NCC=C)C(=O)C=C(NC(=O)/C(C)=C/C=C\[C@@H]1OC)C2=O ; *NC(=O)O[C@H]1/C(C)=C/[C@H](C)[C@@H](O)[C@@H](OC)C[C@H](C)CC2=C(NCCN(C)C)C(=O)C=C(NC(=O)/C(C)=C/C=C\[C@@H]1OC)C2=O]

or an isotope of one or more chemical elements, or pharmaceutically acceptable salts, hydrates, or hydrated salts; or the polymorphic crystalline structures of these compounds; or the optical isomers, diastereomers or enantiomers;
wherein [symbol] is the site linked to L₁ or L₂;
In certain such embodiments, Maytansines or their derivatives maytansinoids inhibit cell proliferation by inhibiting the mcirotubules formation during mitosis through inhibition of polymerization of tubulin, and are peferably the cytotoxic agent of the present invention. See Remillard et al., Science 189:1002-1005 (1975). Exemplary maytansines and maytansinoids include, but are not limited to, mertansines (DM1, DM4), maytansinol and its derivatives as well as ansamitocin. Maytansinoids are described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333,410; 6,441,163; 6,716,821, 7,276,497, 7,301,019, 7,303,749, 7,368,565, 7,411,063, 7,851,432, and 8,163,888. The structure of maytansinoids is preferred the following formula:

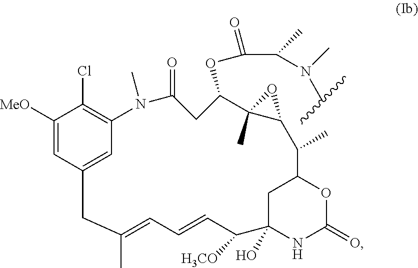 [SMILES: *N(C)[C@@H](C)C(=O)O[C@H]1CC(=O)N(C)c2cc(cc(OC)c2Cl)C/C(C)=C/C=C/[C@@H](OC)[C@@]2(O)CC(OC(=O)N2)[C@@H](C)[C@H]2O[C@]12C]

or an isotope of one or more chemical elements, or pharmaceutically acceptable salts, hydrates, or hydrated salts; or the polymorphic crystalline structures of these compounds; or the optical isomers, diastereomers or enantiomers;
wherein [symbol] is the site linked to L₁ or L₂.

In certain such embodiments, cryptophycin or their derivatives are peferably the cytotoxic agent of the present invention. The cryptophycins are a family of 16-membered macrolide antimitotic agents isolated from the cyanobacteria Nostoc sp. The mechanism of anticancer activity of the cryptophycins has been associated with their destabilization of microtubules and induction of bcl-2 phosphorylation leading to apoptosis. Cryptophycins demonstrated activity against the wide spectrum of solid tumors including those that overexpress the multidrug resistance efflux pump P-glycoprotein.

The structures of cryptophycins of this invention are preferred the following formula:

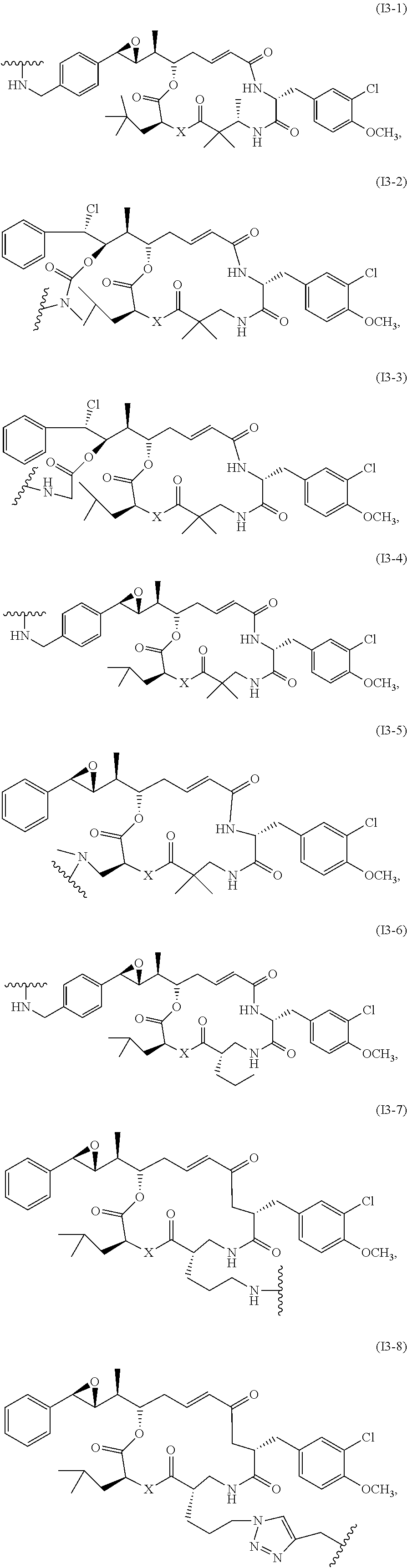

or an isotope of one or more chemical elements, or pharmaceutically acceptable salts, hydrates, or hydrated salts; or the polymorphic crystalline structures of these compounds; or the optical isomers, diastereomers or enantiomers;
wherein X is O or NH, and [symbol] is the site linked to L₁ or L₂.

In certain such embodiments, camptothecins (CPTs) and its derivatives are preferably the cytotoxic agent of the present invention. CPTs are topoisomerase I inhibitors to prevent DNA re-ligation and therefore to causes DNA damage resulting in apoptosis, and are described in: Shang, X. F. et al, Med Res Rev. 2018, 38(3):775-828; Botella, P. and Rivero-Buceta, E. J Control Release. 2017, 247:28-54; Martino, E. et al, Bioorg Med Chem Lett. 2017, 27(4):701-707; Lu, A., et al, Acta Pharmacol Sin 2007, 28(2): 307-314. It includes SN-38, Topotecan, Irinotecan (CPT-11), Silatecan (DB-67, AR-67), Cositecan (BNP-1350), Etirinotecan, Exatecan, Lurtotecan, Gimatecan (ST1481), Belotecan (CKD-602), Rubitecan and several others (Shang, X. F. et al, Med Res Rev. 2018, 38(3):775-828). So far three CPT analogues, topotecan, irinotecan, and belotecan have been approved and are used in cancer chemotherapy (Palakurthi, S., Expert Opin Drug Deliv. 2015; 12(12):1911-21; Shang, X. F. et al, Med Res Rev. 2018, 38(3):775-828) and both SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21 (17): 3870-8; Cardillo, T. M., et al, Bioconjug Chem. 2015, 26 (5): 919-31; Ogitani, Y. et al, Bioorg Med Chem Lett. 2016, 26(20): 5069-5072; Takegawa, N. et al, Int J Cancer. 2017 Oct. 15; 141 (8): 1682-1689. U.S. Pat. Nos. 7,591,994; 7,999,083, 8,080,250, 8,268,317; U.S. patent applications 20130090458, 20140099258, 20150297748, 20160279259).

The structure of Camptothecin (CPT) used for the patent is illustrated below formula:

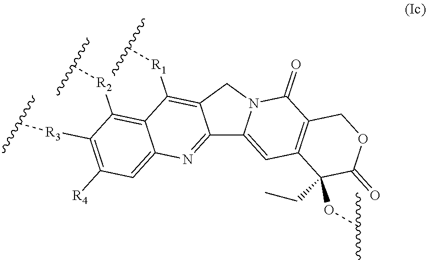

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁˜C₈ alkyl; O-C₁˜C₈ alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or C₂-C₈ of esters, ether, amide, carbonate, urea, or carbamate; R₃ is H, OH, NH₂, C₁˜C₈ alkyl; O-C₁˜C₈ alkyl; NH—C₁˜C₈ alkyl; C₂-C₈ of heteroalkyl, alkylcycloalkyl, heterocycloalkyl, O-C₂˜C₈ alkylamine, NH—C₂˜C₈ alkylamine, O-C₂˜C₈ alkylalcohol, NH—C₂˜C₈ alkylalcohol; or C₂˜C₈ (2-8 carbon atoms) of esters, ether, amide, carbonate, urea, or carbamate; or R₁R₂, R₂R₃ and R₃R₄ independently form a 5˜7 membered carbocyclic, heterocyclic, heterocycloalkyl, aromatic or heteroaromatic ring system; [symbol] is the site in the molecule that can be linked to L₁ or L₂.

The structures of camptothecins are preferred the following formula:

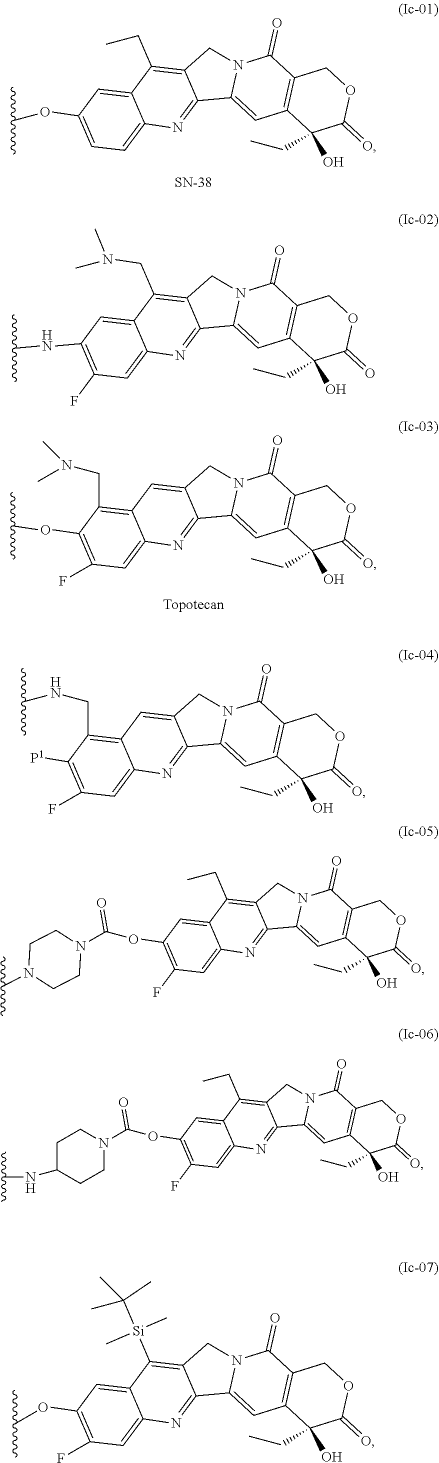

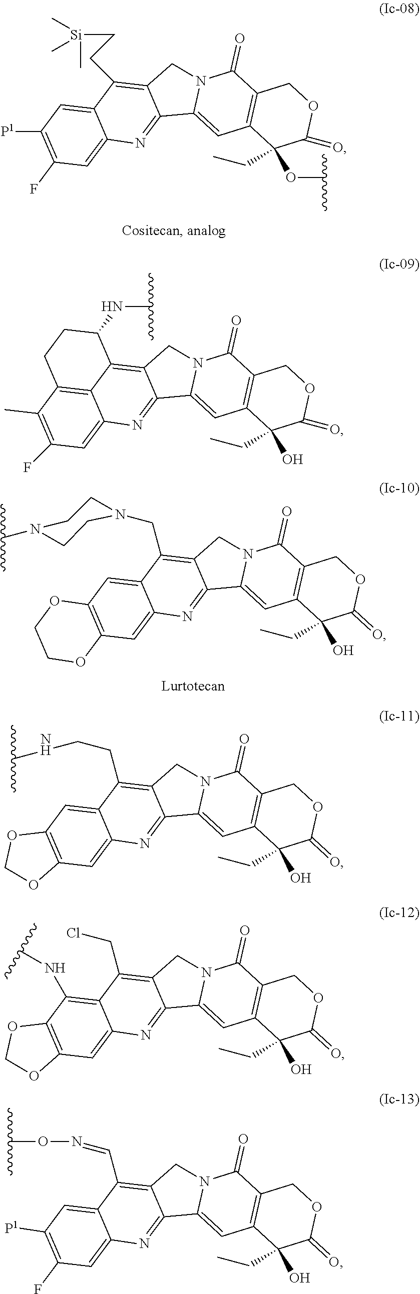

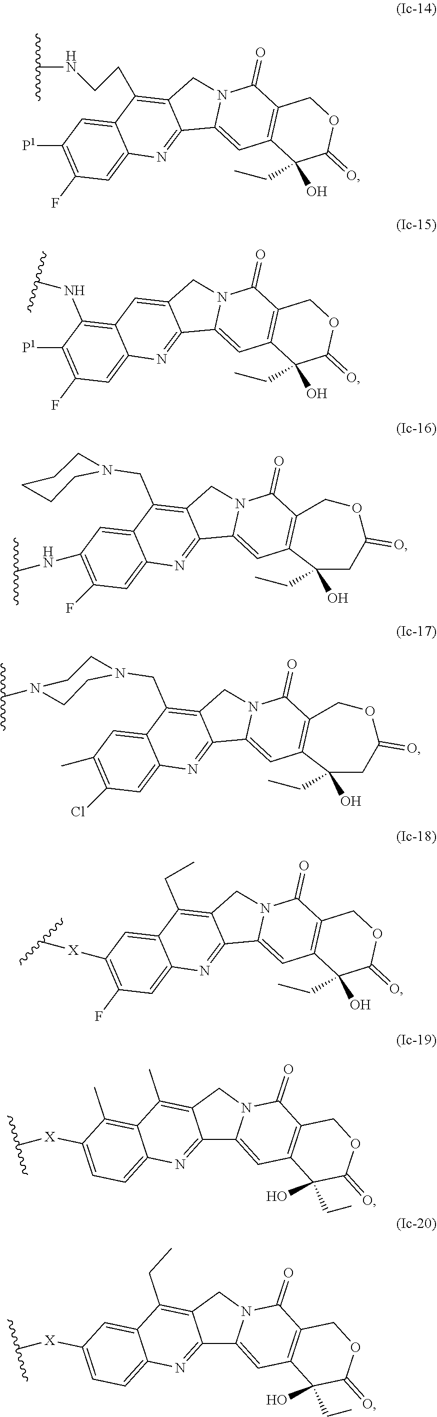

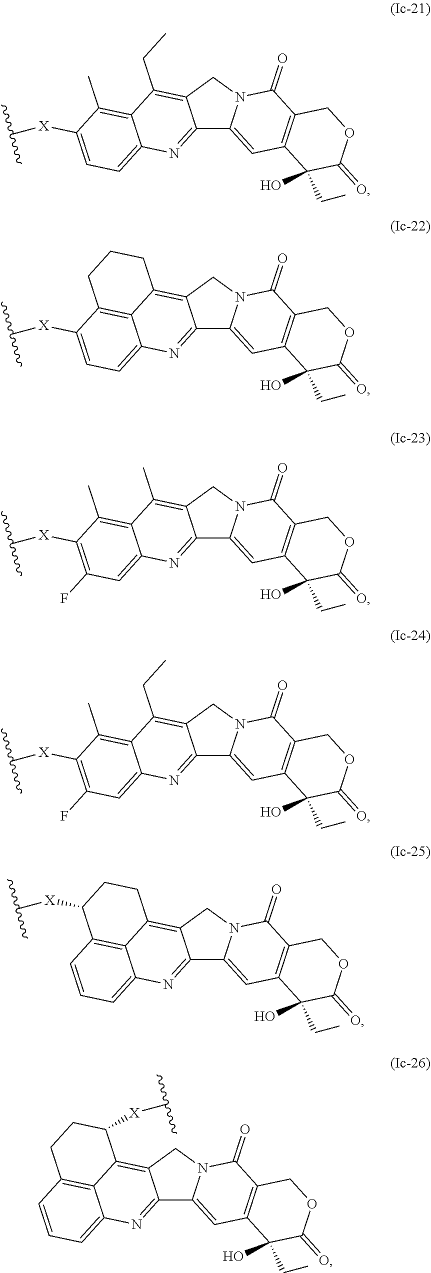

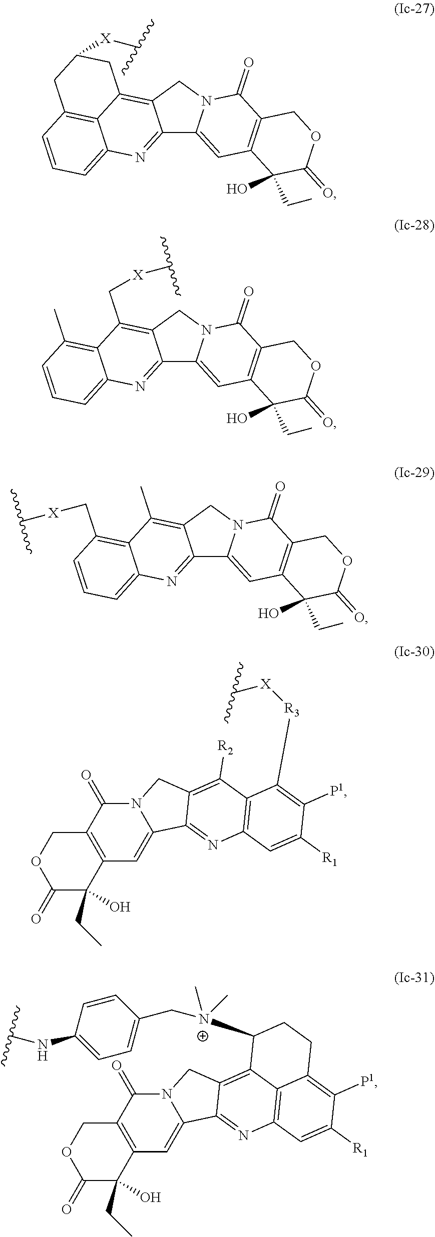

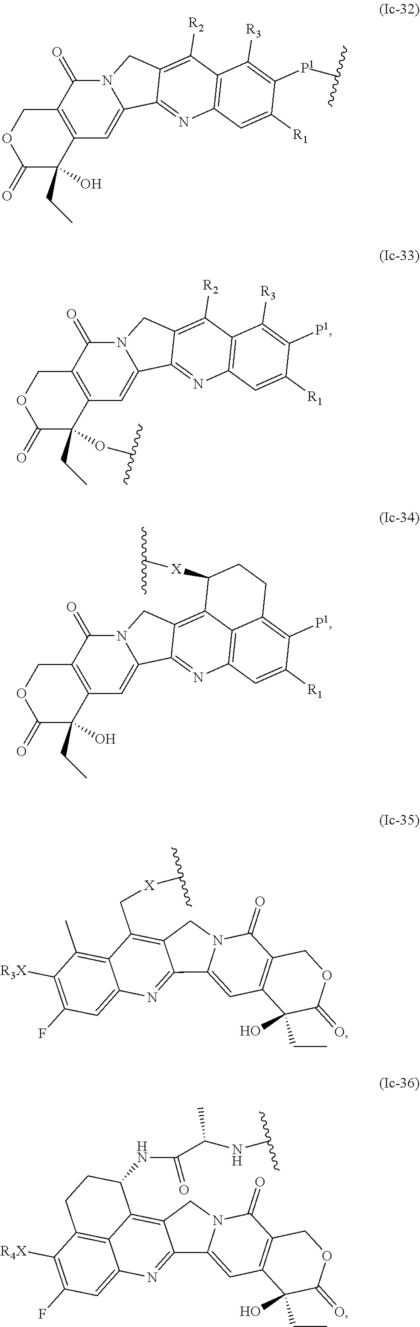

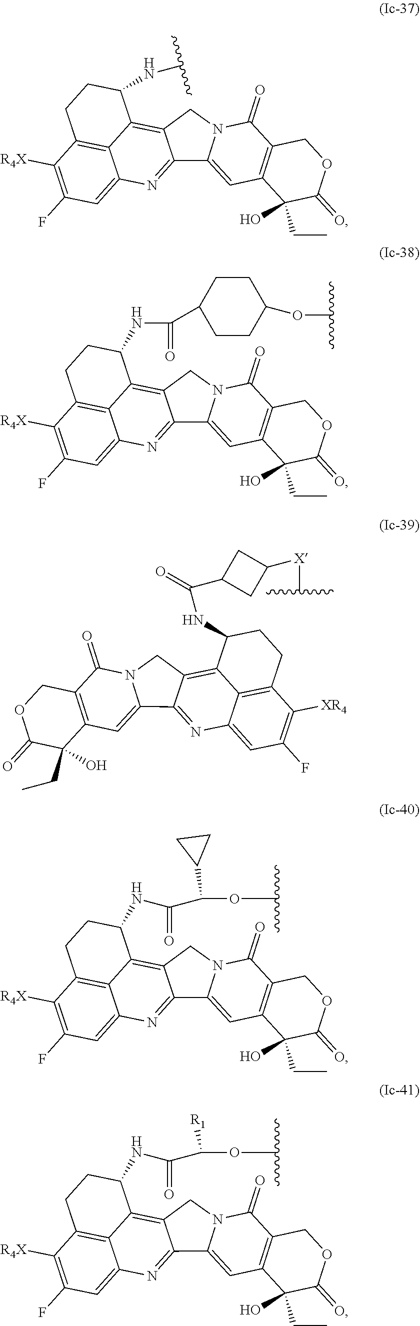

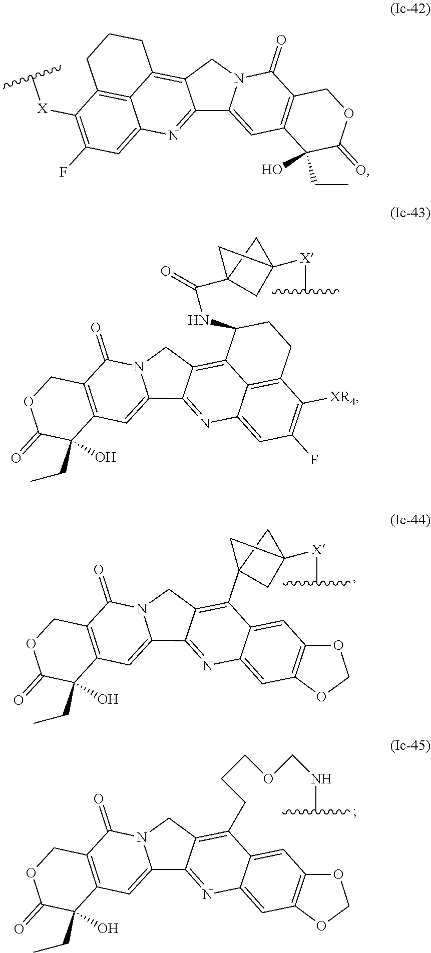

or hydrated salts; or the polymorphic crystalline structures of these compounds; or the optical isomers, racemates, diastereomers or enantiomers; wherein [symbol] is the site linked to L₁ or L₂;
Wherein P¹ and R₁ are independently H, F, Cl, Br, I, SO₂R₂, SO₃H, CN, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R₁₈, OP(O)(OR¹⁸) OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), O—(C₁-C₈ of linear or branched alkyl)-OH, C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R₁₈);
R₂, R₃ and R₄ are independently H, C₁-C₈ of linear or branched alkyl, or heteroalkyl, C₂-C₈ of linear or branched ether, amine, ester, or amide; in addition, R₂ and R₃ can be joint together to form five or six member ring or cycloalkyl, cycloalkylamine, or cycloalkylamide, cyclohexylalkylamide; R₁₇ and R₁₈ are independently H, linear or branched alkyl or heteroalkyl;
X is NH, O, S, S(O₂), NHS(O₂), NHS(O₂)NH, NHP(O)(OH), N⁺(R₂)(R₃), NHC(O)NH, NHC(O), NHC(O)O, N(CH₂CH₂)₂N, CON(CH₂CH₂)₂N, CON(CH₂CH₂)₂N⁺(R₂)(R₃), NHCH₂, N(CH₃), OCH₂, or CH₂; X′ is O, NH, CH₂, CH₂NH, or CH₂O.

In certain such embodiments, Combretastatins are natural phenols with vascular disruption properties in tumors and are preferably the cytotoxic agent of the invention. Exemplary combretastatins and their derivatives include, but are not limited to, combretastatin A-4 (CA-4), CA4-βGals, CA-4PD, CA4-NPs and ombrabulin, having the following formula:

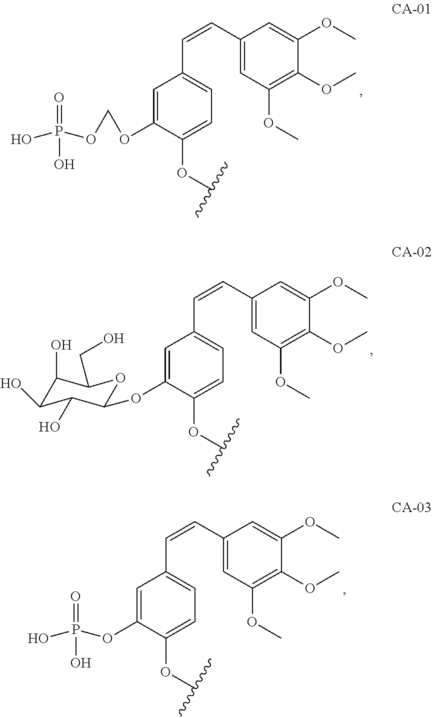 [SMILES: *Oc1ccc(/C=C\c2cc(OC)c(OC)c(OC)c2)cc1OCOP(=O)(O)O ; *Oc1ccc(/C=C\c2cc(OC)c(OC)c(OC)c2)cc1OC1OC(CO)C(O)C(O)C1O ; *Oc1ccc(/C=C\c2cc(OC)c(OC)c(OC)c2)cc1OP(=O)(O)O]

or an isotope of one or more chemical elements, or pharmaceutically acceptable salts, hydrates, or hydrated salts; or the polymorphic crystalline structures of these compounds; or the optical isomers, diastereomers or enantiomers;
In certain such embodiments, Taxanes, which includes Paclitaxel (Taxol), a cytotoxic natural product, and docetaxel (Taxotere), a semi-synthetic derivative, and their analogs, are preferred for conjugation. Taxanes are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Pat. No. 5,475,011 5,728,849, 5,811,452; 6,340,701; 6,372,738; 6,391,913, 6,436,931; 6,589,979; 6,596,757; 6,706,708; 7,008,942; 7,186,851; 7,217,819; 7,276,499; 7,598,290; and 7,667,054. The structures of taxanes are preferred the following formula:

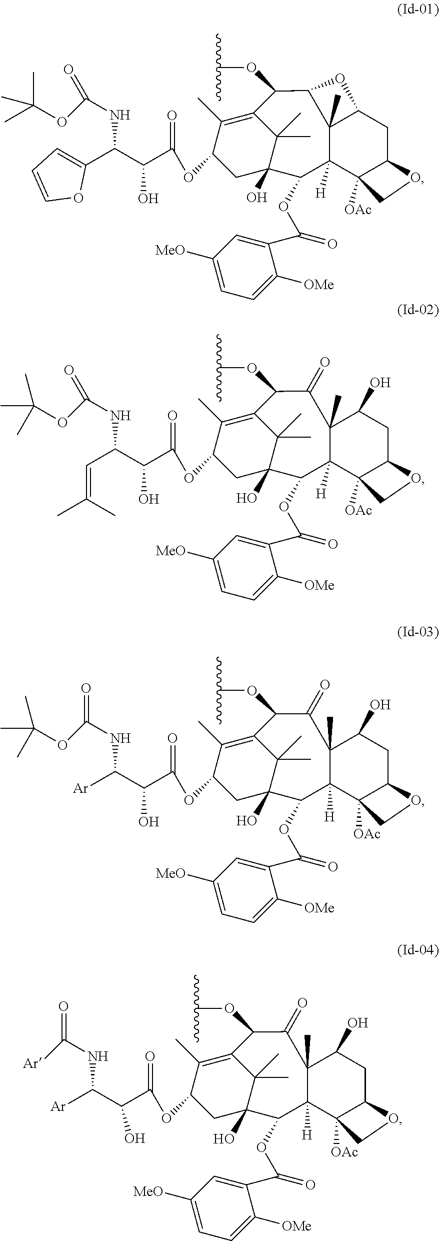

or hydrated salts; or the polymorphic crystalline structures of these compounds; or the optical isomers, diastereomers or enantiomers;
wherein [symbol] is the site linked to L₁ or L₂; Ar and Ar′ are independently aryl or heteroaryl.

In certain such embodiments, Anthracyclines are preferably the cytoxic agents of the invention. Anthracyclines are mammalian DNA topoisomerases II inhibitors that are able to stabilize enzyme-DNA complexes wherein DNA strands are cut and covalently linked to the antibody. These anticancer agents maintain a prominent role in treating many forms of solid tumors and acute leukemias during the last several decades. However, anthracyclines cause cardiovascular morbidity and mortality (Sagi, J. C., et al, Pharmacogenomics. 2016, 17(9), 1075-87; McGowan, J. V., et al, Cardiovasc Drugs Ther. 2017, 31(1), 63-75). Thus, to enhance specific activity of such molecules while reducing the cardiotoxicity, reasearchers actively are using the conjugation of anthracyclines to a cell-binding antibody, or antibody molecule as a general approach for improving the therapeutic index of these drugs, (Mollaev, M. et al, Int J Pharm. 2018 Dec. 29. pii: S0378-5173(18) 30991-8; Rossin, R., et al, Bioconjug Chem. 2016, 27(7):1697-706; Dal Corso, A., et al, J Control Release. 2017, 264:211-218). Exemplary anthracyclines include, but are not limited to, daunorubicin, doxorubicin (i.e., adriamycin), epirubicin, idarubicin, valrubicin, and mitoxantrone. The structures of anthracyclines used for the present application are preferred the following formula;

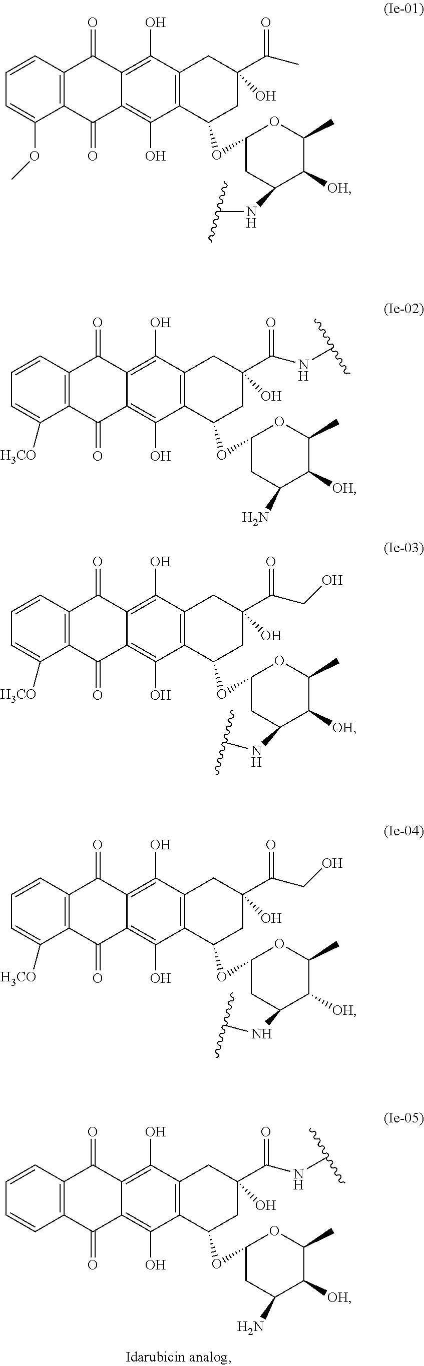

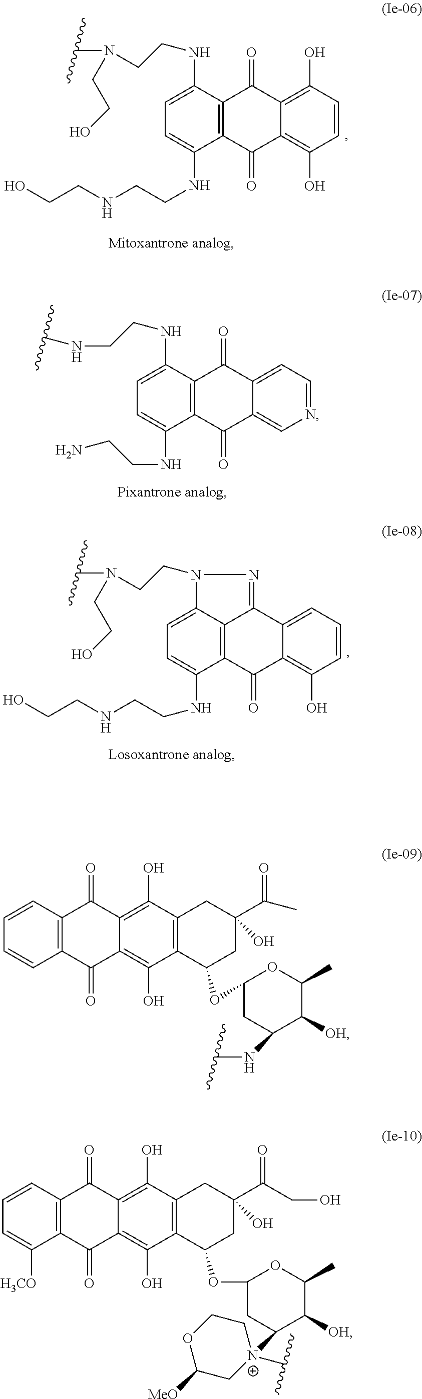 [SMILES: *N(CCO)CCNc1ccc(NCCNCCO)c2c1C(=O)c1c(O)ccc(O)c1C2=O ; *NCCNc1ccc(NCCN)c2c1C(=O)c1ccncc1C2=O ; *N(CCO)CCn1nc2c3c(c(NCCNCCO)ccc31)C(=O)c1c(O)cccc1-2 ; *N[C@H]1C[C@H](O[C@H]2C[C@](O)(C(C)=O)Cc3c(O)c4c(c(O)c32)C(=O)c2ccccc2C4=O)O[C@@H](C)[C@H]1O ; *[N+]1([C@H]2CC(O[C@H]3C[C@](O)(C(=O)CO)Cc4c(O)c5c(c(O)c43)C(=O)c3c(OC)cccc3C5=O)O[C@@H](C)[C@H]2O)CCO[C@H](OC)C1]

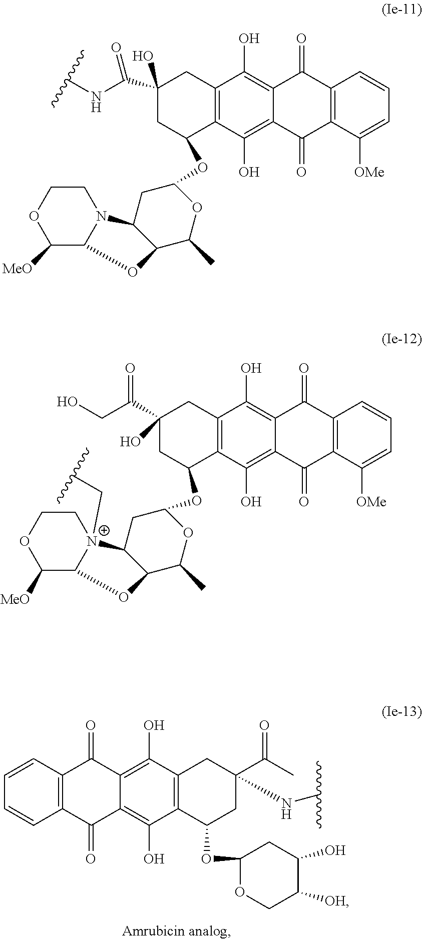

or hydrated salts; or the polymorphic crystalline structures of these compounds; or the optical isomers, diastereomers or enantiomers;
wherein [symbol] is the site that links to L₁ or L₂.

In certain such embodiments, Vinca alkaloids are preferably the cytoxic agents of the invention. Vinca alkaloids are a set of anti-mitotic and anti-microtubule alkaloid agents that work by inhibiting the ability of cancer cells to divide. Vinca alkaloids include vinblastine, vincristine, vindesine, leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine. The structures of vinca alkaloids are preferred vinblastine, vincristine having the following formula:

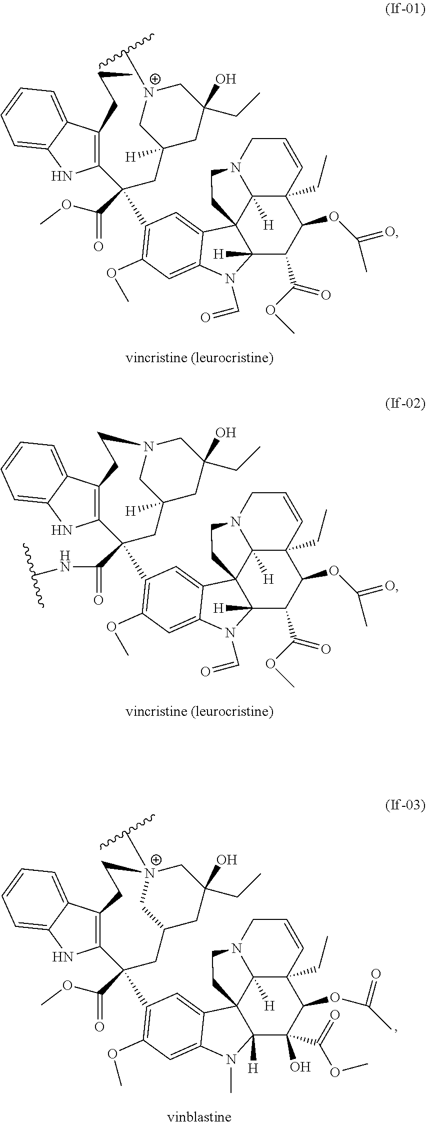

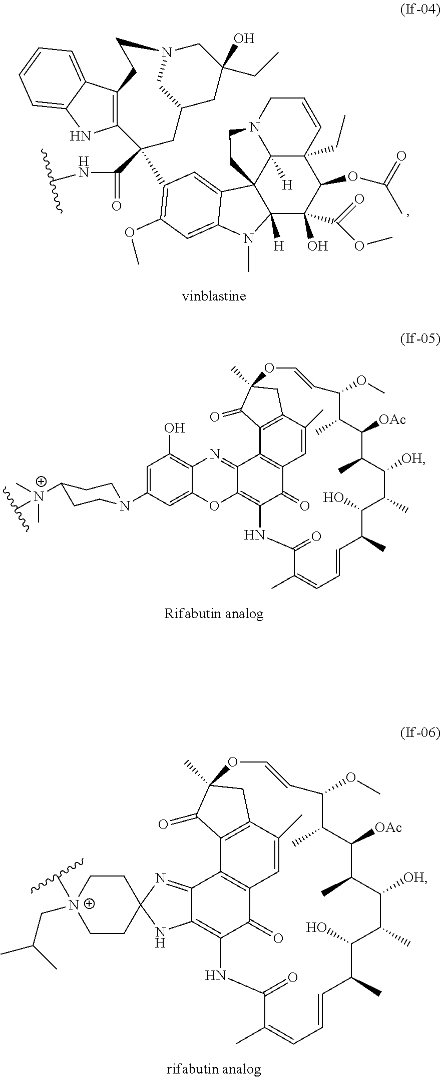

or hydrated salts; or the polymorphic crystalline structures of these compounds; or the optical isomers, diastereomers or enantiomers;
wherein [symbol] is the site linked to L₁ or L₂;
In certain such embodiments, Dolastatins and their peptidic analogs and derivatives, auristatins, are preferably the cytoxic agents of the invention. They are highly potent antimitotic agents that have been shown to have anticancer and antifungal activity. See, e.g., U.S. Pat. No. 5,663,149 and Pettit et al., Antimicrob. Agents Chemother. 42:2961-2965, 1998. Exemplary dolastatins and auristatins include, but are not limited to, dolastatin 10, auristatin E (AE), auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), Auristatin F phenylene diamine (AFP) and other novel auristatins. The auristatins are described in Int. J. Oncol. 15:367-72 (1999); Molecular Cancer Therapeutics, vol. 3, No. 8, pp. 921-32 (2004); U.S. Application Nos. 11134826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. The structures of auristatin analogs are preferred the following formula (Ih-01), (Ih-02), (Ih-03), (Ih-04), (Ih-05), (Ih-06), (Ih-07), (Ih-08), (Ih-09), (Ih-10), and (Ih-11):

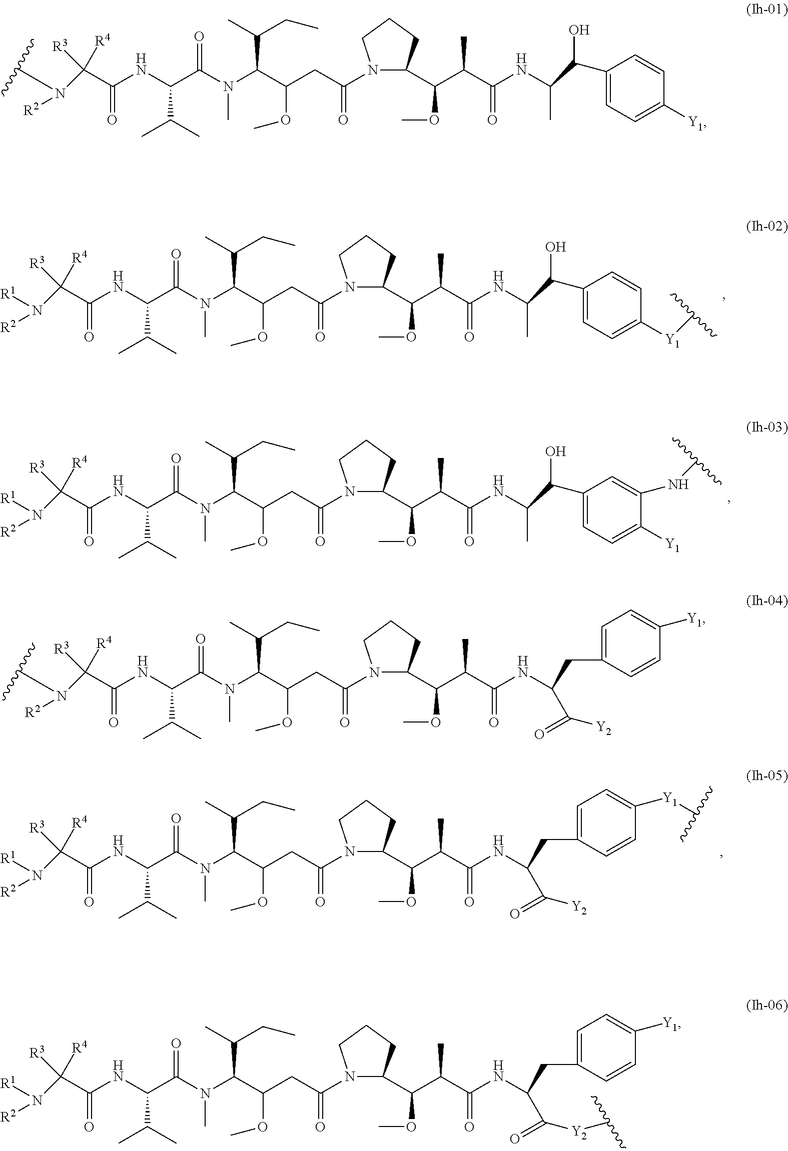

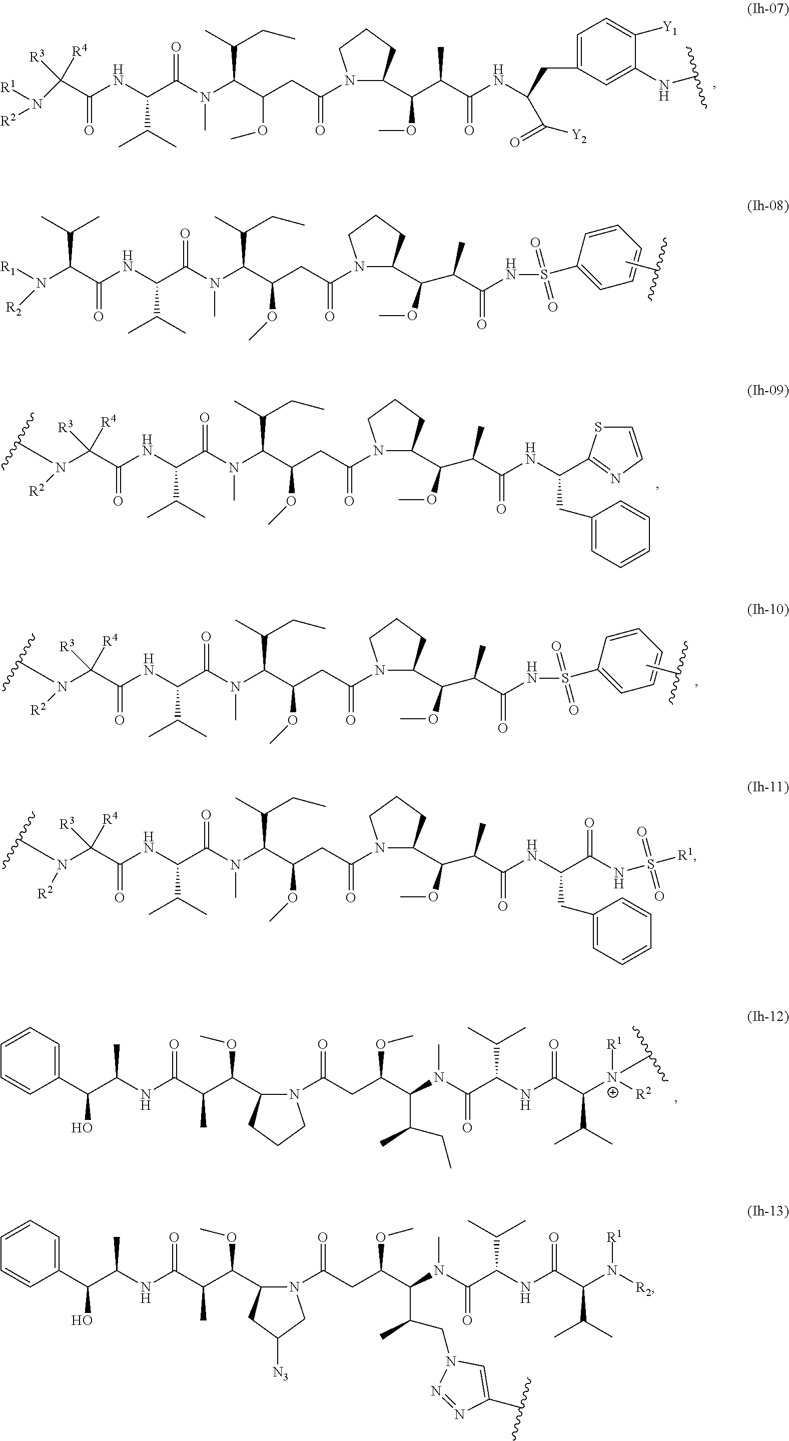

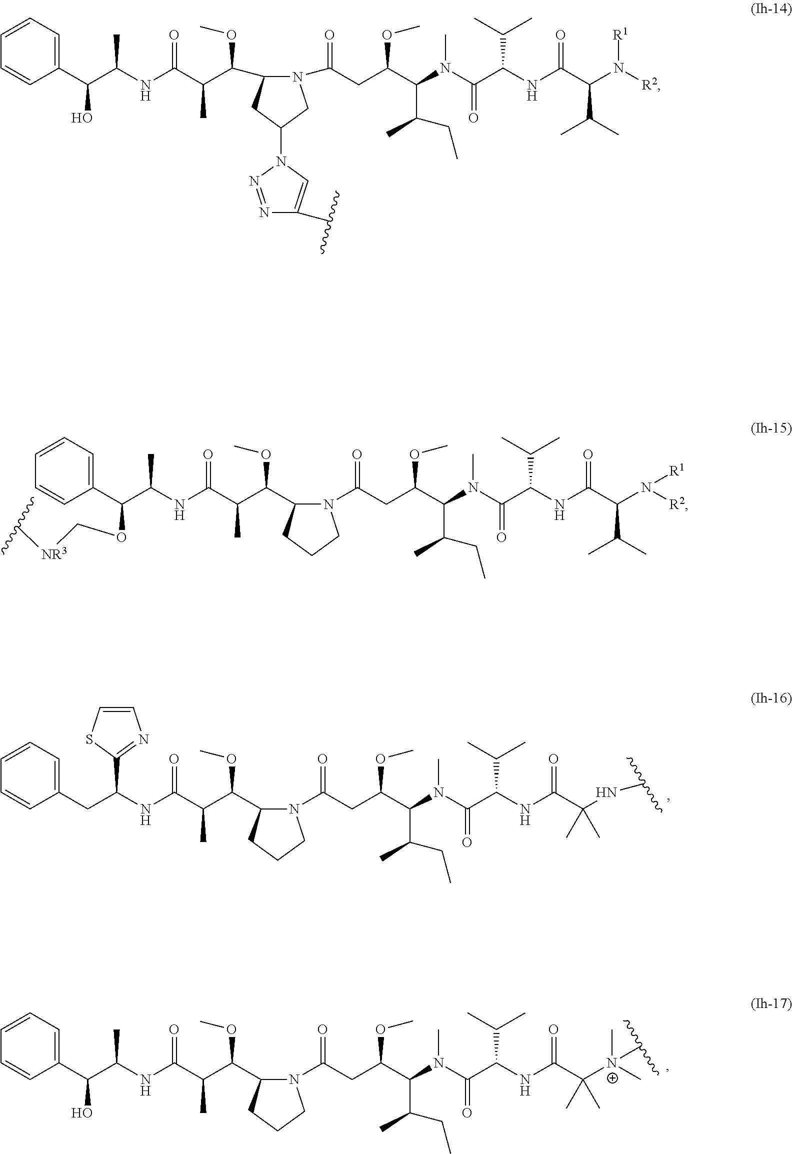

or hydrated salts; or the polymorphic crystalline structures of these compounds; or the optical isomers, diastereomers or enantiomers;
wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 100. The two Rs: R¹R², R²R³, R¹R³ or R³R⁴ together can form 3˜8 member cyclic ring of alkyl, aryl, heteroaryl, heteroalkyl, or alkylcycloalkyl group; Y₁ and Y₂ are independently O, NH, NHNH, NR₅, S, C(O)O, C(O)NH, OC(O)NH, OC(O)O, NHC(O)NH, NHC(O) S, OC(O)N(R₁), N(R₁) C(O)N(R₂), C(O)NHNHC(O) and C(O)NR₁ when linked to the connecting site “ [symbol] ” (that links to L₁ and/or L₂ independently); or OH, NH₂, NHNH₂, NHR⁵, SH, C(O)OH, C(O)NH₂, OC(O)NH₂, OC(O)OH, NHC(O)NH₂, NHC(O) SH, OC(O)NH(R₁), N(R₁) C(O)NH(R₂), C(O)NHNHC(O)OH and C(O)NHR¹ when not linked to the connecting site “ [symbol] ”; R₁₂ is OH, NH₂, NHR₁, NHANH₂, NHANHCOOH, O—R₁—COOH, NH—R¹—COOH, NH-(Aa)ₙCOOH, O(CH₂CH₂O)ₚCH₂CH₂OH, O(CH₂CH₂O)ₚCH2CH₂NH₂, NH(CH₂CH₂O)ₚCH2CH₂NH₂, NR₁R₁′, NHOH, NHOR₁, O(CH₂CH₂O)ₚCH2CH₂COOH, NH(CH₂CH₂O)ₚCH2CH₂COOH, NH—Ar—COOH, NH—Ar—NH₂, O(CH₂CH₂O)ₚCH2CH₂NH—SO₃H, NH(CH₂CH₂O)ₚCH2CH₂NHSO₃H, R¹—NHSO₃H, NH—R¹—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-100; R₁, R₂, R₃, R₄, R₅, R₅′, Z¹, Z², and n are defined the same above.

Preferably R₁, R₂, R₃, R₄, R₅, R₅′ are independently H, C₁-C₈ alkyl, alkylcarbonyl; Y₁ and Y₂ are independently O, NH, NHNH, NR₅, C(O)O, C(O)NH, OC(O)NH, OC(O)O, NHC(O)NH, C(O)NHNHC(O), C(O)NR₁ OH, NH₂, NHNH₂, NHR₅, C(O)OH, C(O)NH₂, OC(O)NH₂, OC(O)OH, NHC(O)NH₂.

In certain such embodiments, Hemiasterlin and its analogues (e.g., HTI-286) are preferably the cytoxic agents of the invention. They bind to the tubulin, disrupt normal microtubule dynamics, and, at stoichiometric amounts, depolymerize microtubules. The structures of hemiasterlins are preferred the following formula:

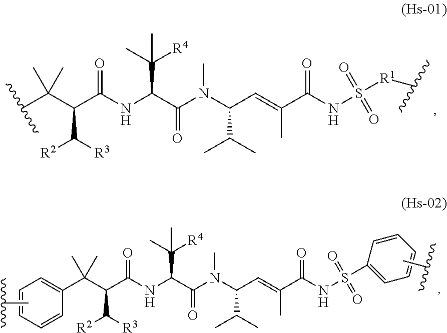

or an isotope of one or more chemical elements, or pharmaceutically acceptable salts, hydrates, or hydrated salts; or the polymorphic crystalline structures of these compounds; or the optical isomers, diastereomers or enantiomers;
wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 100; In addition, R₂R₃ can form 3˜8 member cyclic ring of alkyl, aryl, heteroaryl, heteroalkyl, or alkylcycloalkyl group.

In certain such embodiments, Eribulin is preferably the cytoxic agents of the invention. Eribulin is binding predominantly to a small number of high affinity sites at the plus ends of existing microtubules and has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6. Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). The structure of Eribulin is preferred the following formula, Eb01:

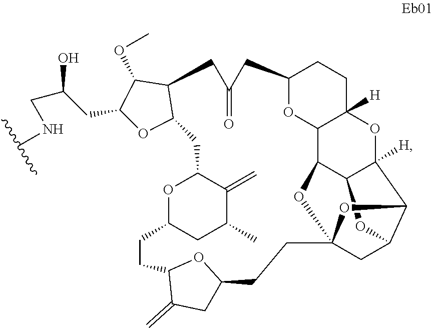

[symbol] is a linkage site that links to L₁ and/or L₂ independently;
In certain such embodiments, an Inhibitor of nicotinamide phosphoribosyltransferases (NAMPT) can be cytotoxic agent of the invention due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus using ADCs for targeting delivery of NAMPT inhibitors might circumvent the systemic toxicities to achieve much broader therapeutic index. The structures of NAMPT inhibitors are preferred the following formula, NP01, NP02, NP03, NP04, NP05, NP06, NP07, NP08, and NP09:

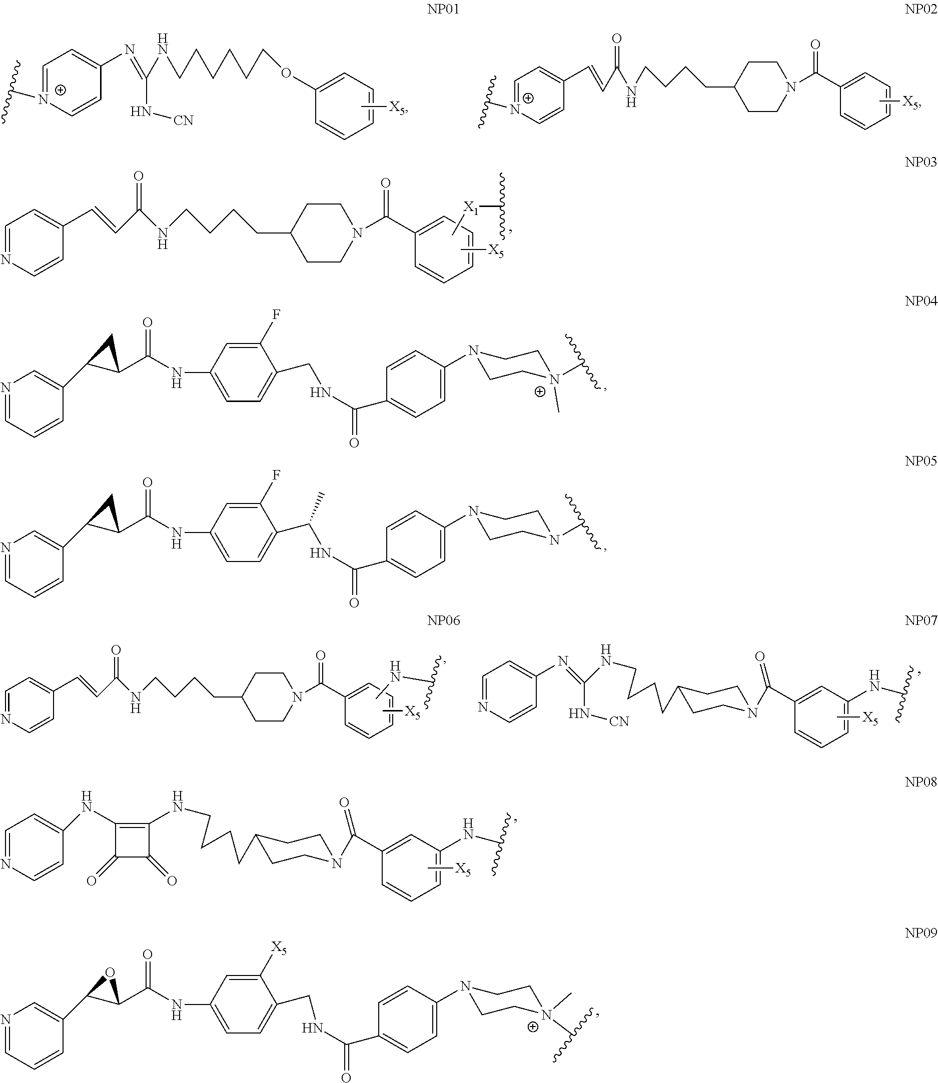

or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OSO₃H₂, OSO₃H, NHR₁, OCOR₁, NHCOR₁; R₁ is H, C₁˜C₈ alkyl, CH₃CO, NH, NH₂, NHCH₃;
[symbol] is a linkage site that links to L₁ and/or L₂ independently;
In certain such embodiments, a benzodiazepine dimer and its analogs are preferably the cytoxic agents of the invention. The benzodiazepine dimers, including: a dimer of pyrrolobenzodiazepine (PBD) or (tomaymycin), a dimer of indolinobenzodiazepine (IGN), a dimer of imidazobenzothiadia-zepine, or a dimer of oxazolidinobenzodiazepines, are anti-tumor agents that contain one or more immine functional groups, or their equivalents, that bind to duplex DNA. PBD and IGN molecules are based on the natural product athramycin, and interact with DNA in a sequence-selective manner, with a preference for purine-guanine-purine sequences. The preferred benzodiazepine dimers according to the present invention are exampled in: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109, 193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; U.S. patent application No. 20100203007, 20100316656, 20030195196. Examples of the structures of the conjugate of the antibody-benzodiazepine dimers are illustrated below PB01˜PB30:

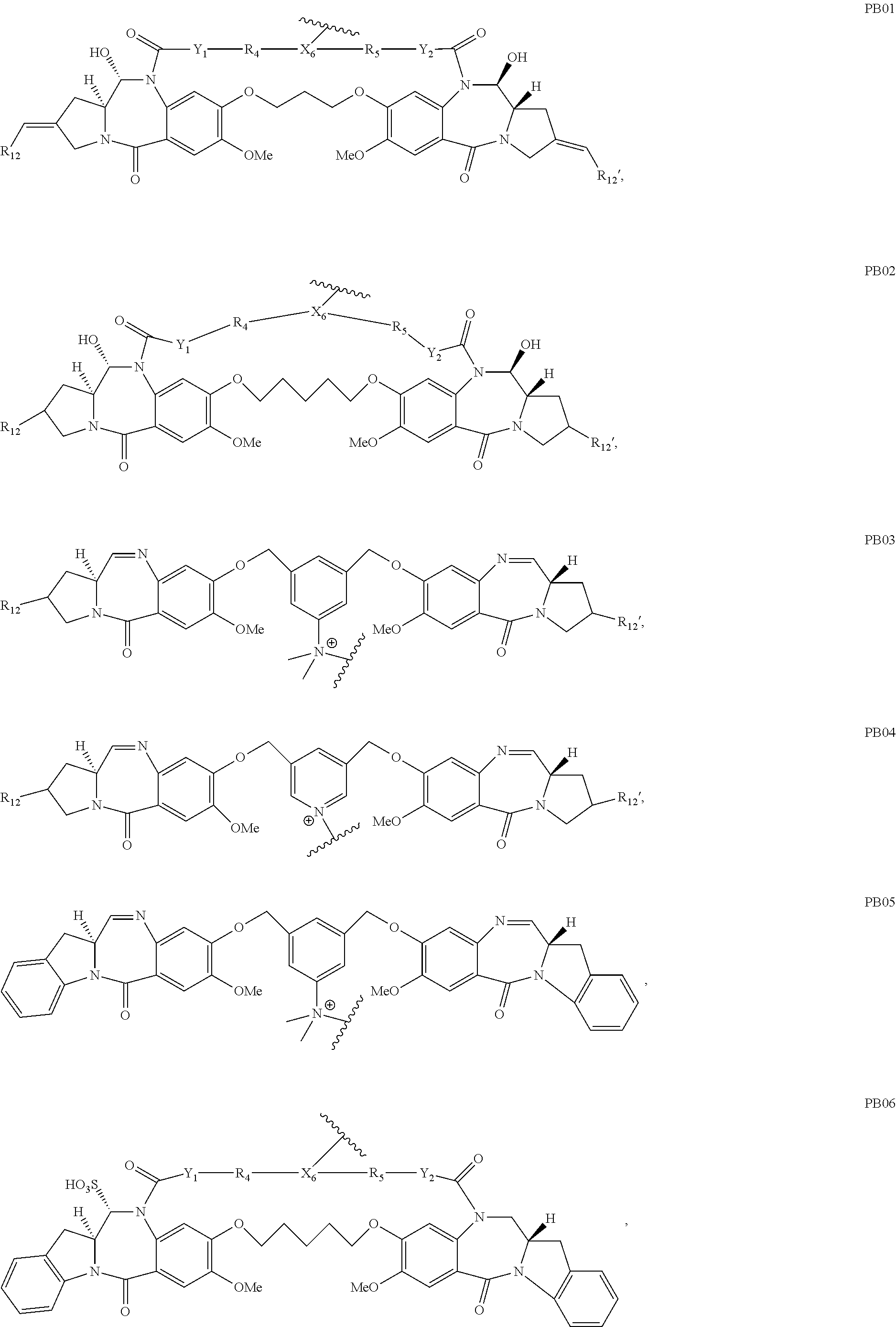

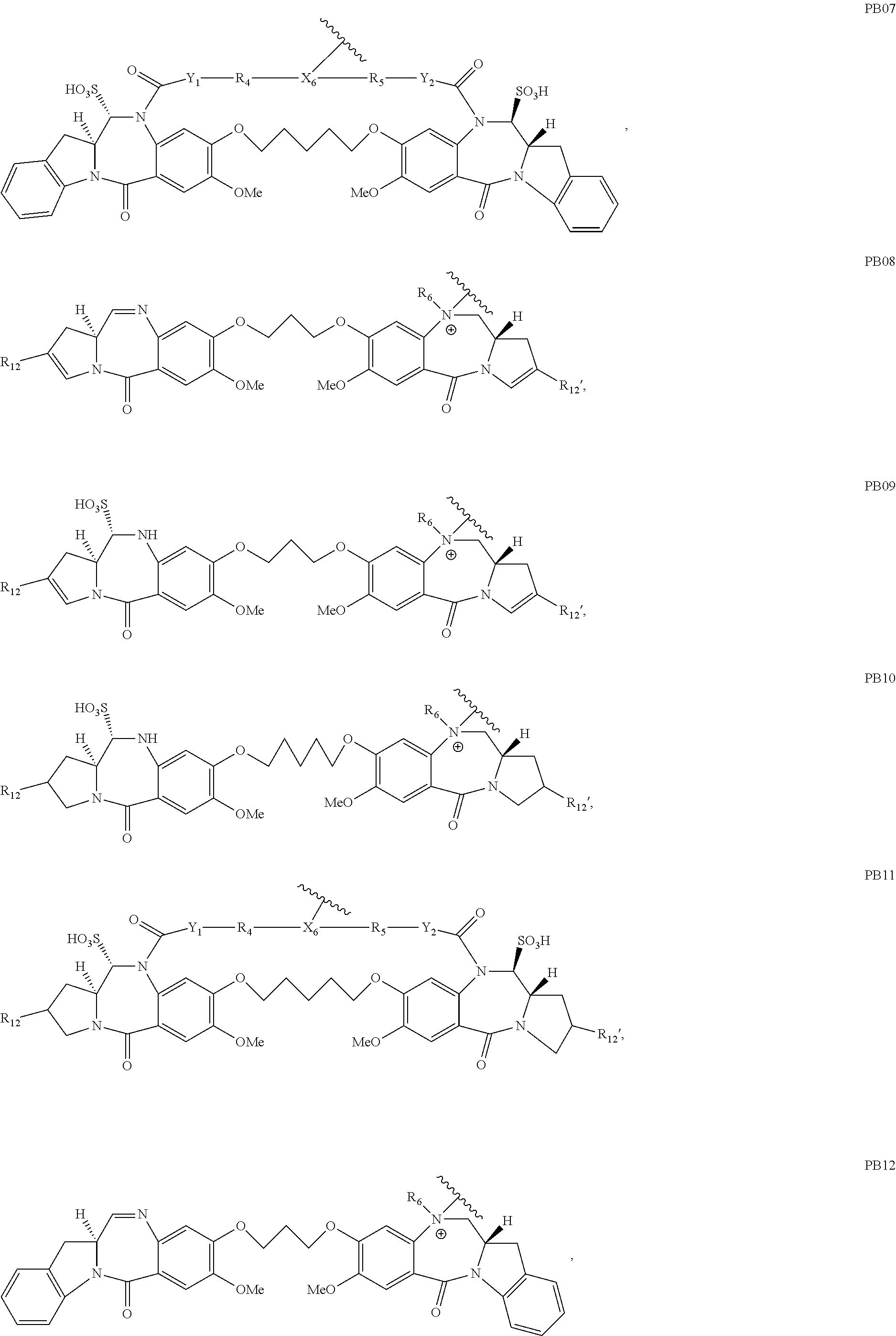

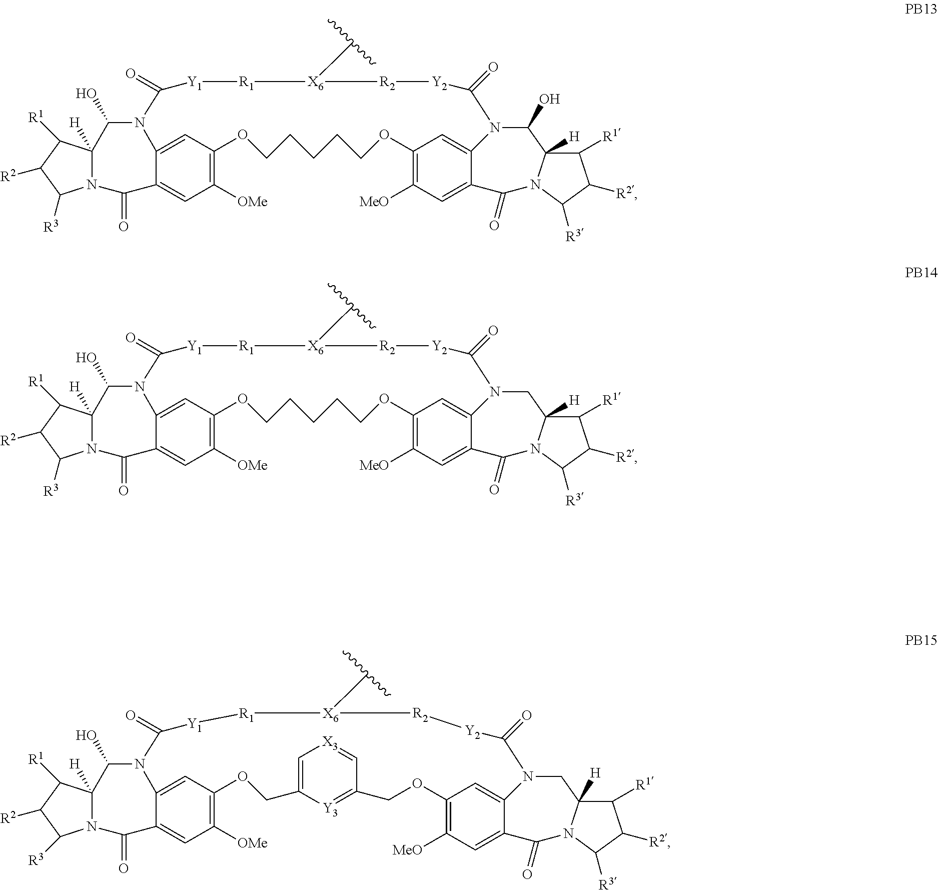

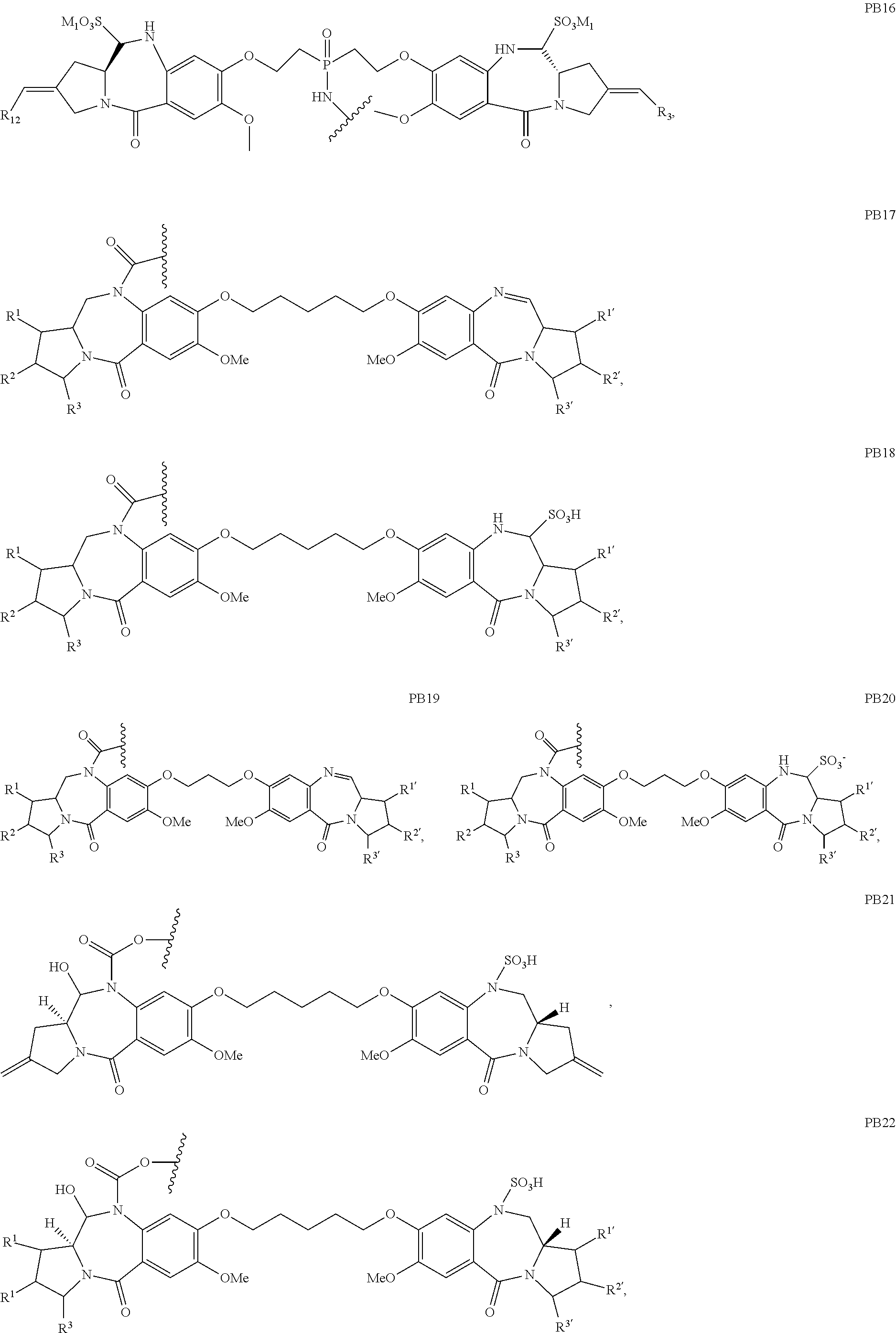

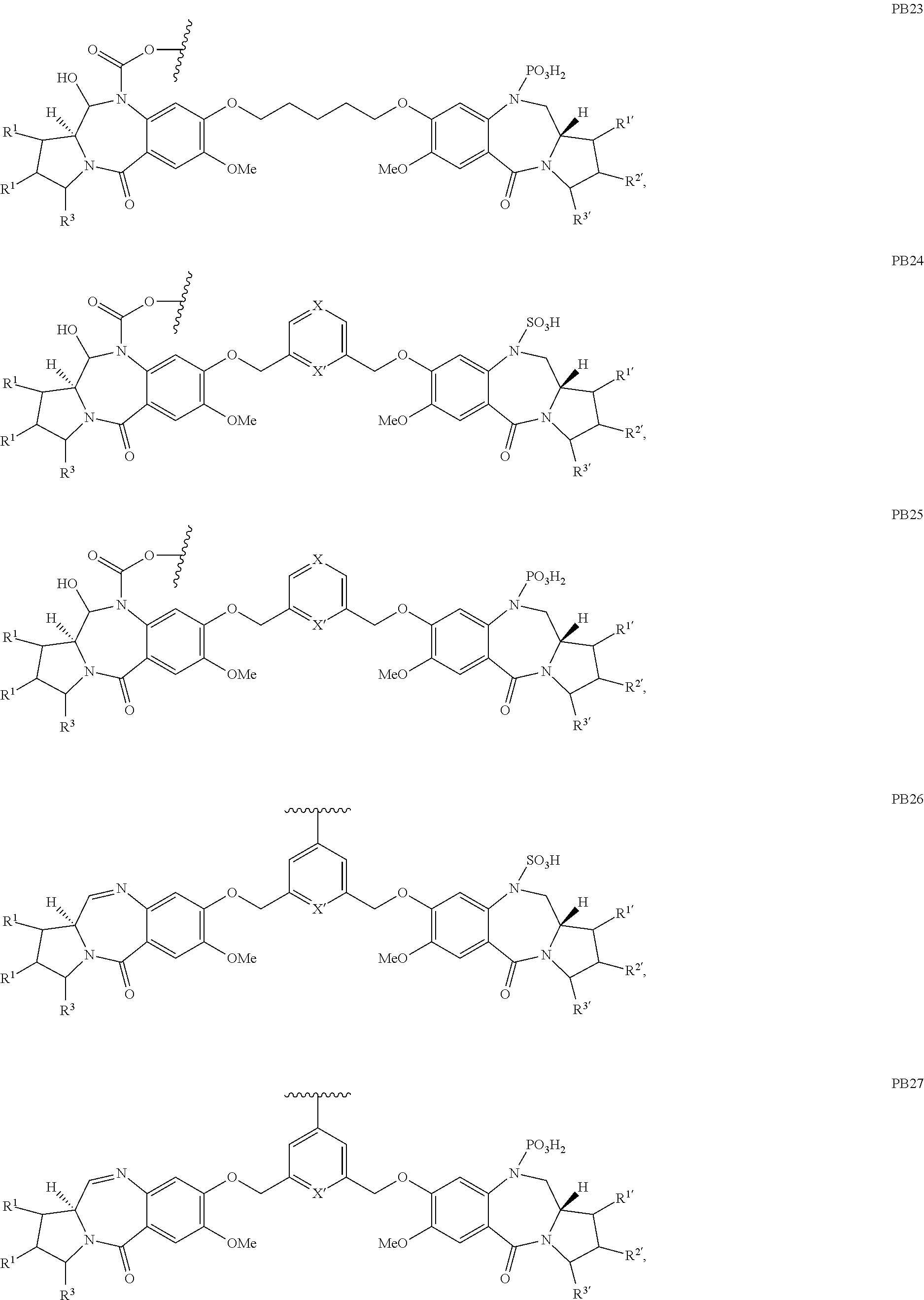

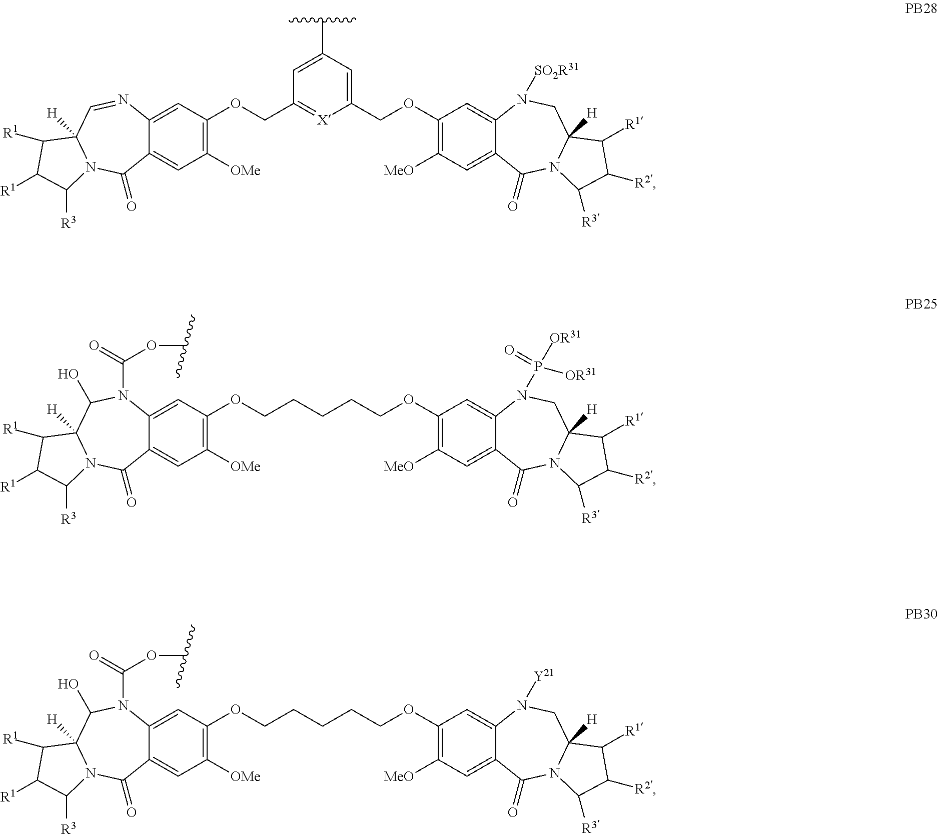

or hydrated salts; or the polymorphic crystalline structures of these compounds; or the optical isomers, racemates, diastereomers or enantiomers; wherein X₁, X₂, Y₁, Y₂, Z¹, Z², and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O) S, OC(O)N(R₁), N(R₁) C(O)N(R₁), CH, C(O)NHNHC(O) and C(O)NR₁;
R¹, R², R³, R^{1′}, R^{2′}, and R^{3′} are independently H; F; Cl; ═O; ═S; ═CH₂; ═CH—R₁, OH; SH; C₁-C₈ linear or branched alkyl, aryl, alkenyl, heteroaryl, heteroalkyl, alkylcycloalkyl, ester (COOR₅ or —OC(O)R₅), ether (OR₁), amide (CONR₁), carbamate (OCONR₁), amines (NHR₁, NR₁R₂), heterocycloalkyl, or acyloxylamines (—C(O)NHOH, —ONHC(O)R₁); or peptides containing 1-6 natural or unnatural aminoacids, or polyethyleneoxy unit of formula (OCH₂CH₂)ₚ or (OCH₂CH(CH₃))ₚ, wherein p is an integer from 1 to about 100. The two Rs: R¹R², R²R³, R^{1′}R^{2′}, or R^{2′}R^{3′}, can independently form 3˜8-member cyclic ring of C₂˜C₁₀ alkyl, aryl, heteroaryl, heteroalkyl, or alkylcycloalkyl group;
X₃, X, X′ and Y₃ are independently N, O, S, NH, CH₂ or CR1; or one of X₃, X, X′ and Y₃ can be absent, thus the left X₃, X, X′ or Y₃ can be O, S, NH;
wherein R₁, R₂, R₄, and R₅, are C₁-C₈alkyl, heteroalkyl; C₂-C₈ aminoalkylcarbonyl, alkylaryloxyl, alkylarylamino, alkylarylthiol; or 1-6 the same or different sequence of aminao acid/peptides (Aa)r, r=1, 2, 3, 4, 5, or 6;
wherein R₁₂ and R_{12′} are independently H, OH, NH₂, NH(CH₃), NHNH₂, COOH, SH, OZ₃, SZ₃, F, Cl, or C₁-C₈ linear or branched alkyl, C₃-C₈ aryl, heteroaryl, heteroalkyl, alkylcycloalkyl, acyloxylamines;
X₆ is CH, N, P(O)NH, P(O)NR₁, CHC(O)NH, C₃-C₈ aryl, heteroaryl, alkylcycloalkyl, acyloxyl, alkylaryl, alkylaryloxyl, alkylarylamino, or an Aa (amino acid, it is preferably selected from Lys, Phe, Asp, Glu, Ser, Thr, His, Cys, Tyr, Trp, Gln, Asn, Arg);
Y²¹ is Ms (mesyl), Ts (tosyl) or Tf (trifyl), SO₃H, P(O)(OH)₂, CH₂ (O)P(O)(OH)₂, glycoside;
R³¹ is H, C₁-C₈alkyl or Ar, CF₃; “ [symbol] ” is defined the same above.

In certain such embodiments, a CC-1065 analog and doucarmycin analogs are also preferred to be a cytotoxic agent of the present invention. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6,586,618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837, 7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are illustrated below CC01, CC02, CC03, CC04, CC05, CC06 and CC07:

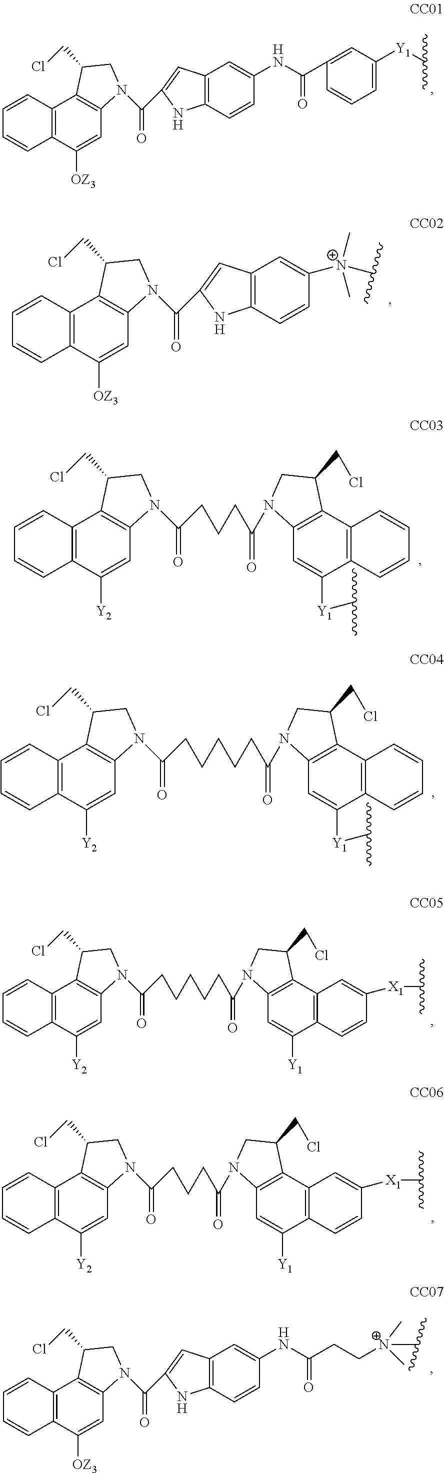

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁ and Y₂ are independently O, NH, NHNH, NR₅, S, C(O)O, C(O)NH, OC(O)NH, OC(O)O, NHC(O)NH, NHC(O) S, OC(O)N(R₁), N(R₁) C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O) SH, OC(O)NH(R₁), N(R₁) C(O)NH(R₂), C(O)NHNHC(O)OH and C(O)NHR₁ when not linked to the connecting site “ [symbol] ”;
Z³ is H, PO(OM₁)(OM₂), SO₃M1, CH₂PO(OM₁)(OM₂), CH₃N(CH₂CH₂)₂NC(O)—, O(CH₂CH₂)₂NC(O)—, R₁, or glycoside; wherein R₁, R₂, and R₃, are C₁-C₈alkyl, heteroalkyl; C₂-C₈ aminoalkylcarbonyl; or 1-4 the same or different sequence of aminao acid/peptides (Aa) r, r=1, 2, 3, 4;
M1, and M2 are pharmaceutical salt selected from Na, K, Ca, NH₄, NH₃CH₂CH₂OH or Zn;
In certain such embodiments, an amatoxin (amanita toxin) and its analogs which are a subgroup of at least ten toxic compounds originally found in several genera of poisonous mushrooms, most notably Amanita phalloides and several other mushroom species, are also preferred as cytotoxic agents of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American232 (3): 90-101; H. E. Hallen, et al, 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15 (16): 3480-4; Fiume, L., Curr Probl Clin Biochem, 1977, 7:23-8; Karlson-Stiber C, Persson H. 2003, Toxicon 42 (4): 339-49; Chafin, D. R., Guo, H. & Price, D. H. 1995 J. Biol. Chem. 270 (32): 19114-19; Wieland (1983) Int. J. Pept. Protein Res. 22 (3): 257-76). Amatoxins can be produced from collected Amanita phalloides mushrooms (Yocum, R. R. 1978 Biochemistry 17 (18): 3786-9; Zhang, P. et al, 2005, FEMS Microbiol. Lett. 252 (2), 223-8), or from fermentation using a basidiomycete (Muraoka, S. and Shinozawa T., 2000 J. Biosci. Bioeng. 89 (1): 73-6) or from fermentation using A. fissa (Guo, X. W., et al, 2006 Wei Sheng Wu Xue Bao 46 (3): 373-8), or from culturing Galerina fasciculata or Galerina helvoliceps, a strain belonging to the genus (WO/1990/009799, JP11137291). However, the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18 (2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870 (3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32 (1): 9-20; G. Zanotti, T. et al, Int. J. Peptide Protein Res. 1989, 34, 222-8; Zanotti, G., et al., Int J Pept Protein Res, 1990. 35 (3): 263-70; Mullersman, J. E. and J. F. Preston, 3rd, Int J Pept Protein Res, 1991. 37 (6): 544-51; Mullersman, J. E., et al, Int J Pept Protein Res, 1991. 38 (5): 409-16; Zanotti, G., et al, Int J Pept Protein Res, 1992. 40 (6): 551-8; Schmitt, W. et al, J. Am. Chem. Soc. 1996, 118, 4380-7; Anderson, M. O., et al, J. Org. Chem., 2005, 70 (12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88 (5): 714-24; May, J. P., et al., Chemistry, 2008. 14 (11): 3410-7; S. De Lamo Marin, et al, Eur. J. Org. Chem. 2010, 3985-9; Pousse, G., et al., Org Lett, 2010. 12 (16): 3582-5; Luo, H., et al., Chem Biol, 2014. 21 (12): 1610-7; Zhao, L., et al., Chembiochem, 2015. 16 (10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243 (130): 281-3; Bonetti, E., M. et al, Arch Toxicol, 1976. 35 (1): p. 69-73; Davis, M. T., Preston, J. F. Science 1981, 213, 1385-1388; Preston, J. F., et al, Arch Biochem Biophys, 1981. 209 (1): 63-71; H. Faulstich, et al, Biochemistry 1981, 20, 6498-504; Barak, L. S., et al., Proc Natl Acad Sci USA, 1981. 78 (5): 3034-8; Faulstich, H. and L. Fiume, Methods Enzymol, 1985. 112:225-37; Zhelev, Z., A. et al, Toxicon, 1987. 25 (9): 981-7; Khalacheva, K., et al, Eksp Med Morfol, 1990. 29 (3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69 (7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16 (10): 1420-5; Zhou, B., et al., Biosens Bioelectron, 2015. 68:189-96; WO2014/043403, US20150218220, EP 1661584). We have been working on the conjugation of amatoxins for a while. Examples of the structures of the amatoxins used for the present application are preferred the following structures of Am01, Am02, and Am03:

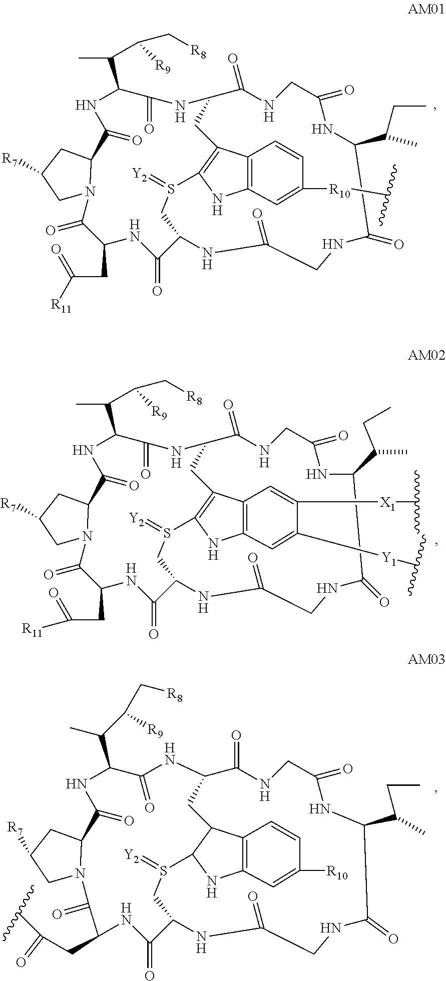

or hydrated salts; or the polymorphic crystalline structures of these compounds; or the optical isomers, racemates, diastereomers or enantiomers; wherein X₁, and Y₁ are independently O, NH, NHNH, NR₁, S, C(O)O, C(O)NH, OC(O)NH, OC(O)O, NHC(O)NH, NHC(O) S, OC(O)N(R₁), N(R₁) C(O)N(R₁), CH₂, CHNH, CH₂O, C(O)NHNHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O) (NR₁), (NR₁) C(O) (NR₁), C(O)R₁ or absent; R₁₁ is OH, NH₂, NHR₁, NHNH₂, NHNHCOOH, O—R₁—COOH, NH—R₁—COOH, NH-(Aa), COOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa) r is r=1, 2, 3 or 4 aminoacids; n and m₁ are independently 1˜20; p is 1-100; R₁, R₂ and Ar, are the same defined throughout the application; and R₁ and Ry are preferably here H, C₁˜C₈ alkyl; “ [symbol] ” is linkage site (which is defined the same above).

In certain such embodiments, spliceostatins and pladienolides are anti-tumor compounds which inhibit splicing and interacts with spliceosome, SF3b, and are also preferred as cytotoxic agents of the present patent. Examples of spliceostatins include, but are not limited to, spliceostatin A, FR901464, and (2S, 3Z)-5-{[(2R, 3R, 5S, 6S)-6-{(2E, 4E)-5-[(3R, 4R, 5R, 7S)-7-(2-hydrazinyl-2-oxoethyl)-4-hydroxy-1, 6-dioxaspiro[2.5] oct-5-yl]-3-methylpenta-2, 4-dien-1-y-1}-2,5-dimethyltetrahydro-2H-pyran-3-yl]amino}-5-oxopent-3-en-2-yl acetate having the core structure of:

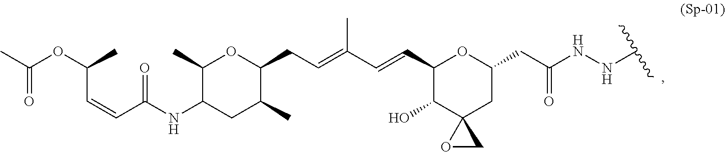 [SMILES: *NNC(=O)C[C@@H]1C[C@@]2(CO2)[C@H](O)[C@@H](/C=C/C(C)=C/C[C@@H]2O[C@H](C)C(NC(=O)/C=C\[C@H](C)OC(C)=O)C[C@@H]2C)O1]

Examples of pladienolides include, but are not limited to, Pladienolide B, Pladienolide D, and E7107.

In certain such embodiments, protein kinase inhibitors are also preferred as cytotoxic agents of the present patent. Protein kinase inhibitors block the action of an enzyme to add a phosphate (PO₄) group to serine, threonine, or tyrosine amino acids on a protein, and can modulate the protein function. The protein kinase inhibitors can be used to treat diseases due to hyperactive protein kinases (including mutant or overexpressed kinases) in cancer or to modulate cell functions to overcome other disease drivers. The structures of protein kinase inhibitors are preferred to selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, Vemurafenib, Entrectinib, Palbociclib, Ribociclib, Abemaciclib, Dacomitinib, Neratinib, Rociletinib (CO-1686), Osimertinib, AZD3759, Nazartinib (EGF816), having the following formula, PK01˜PK40:

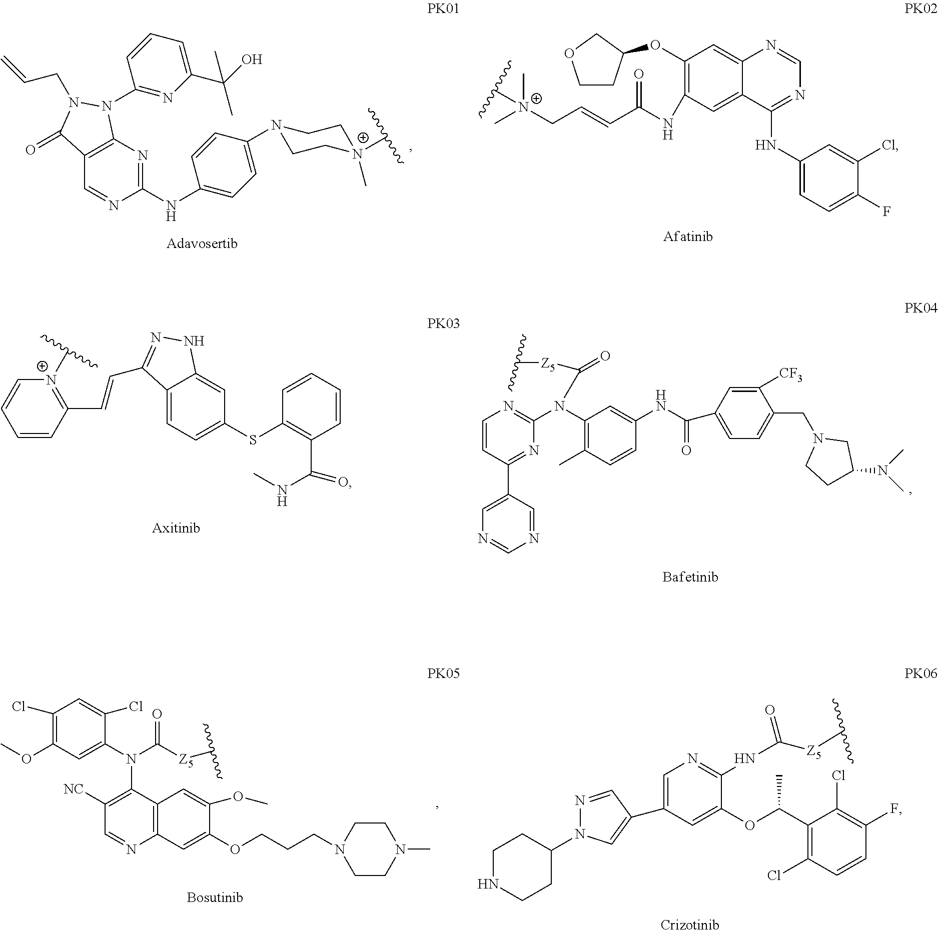

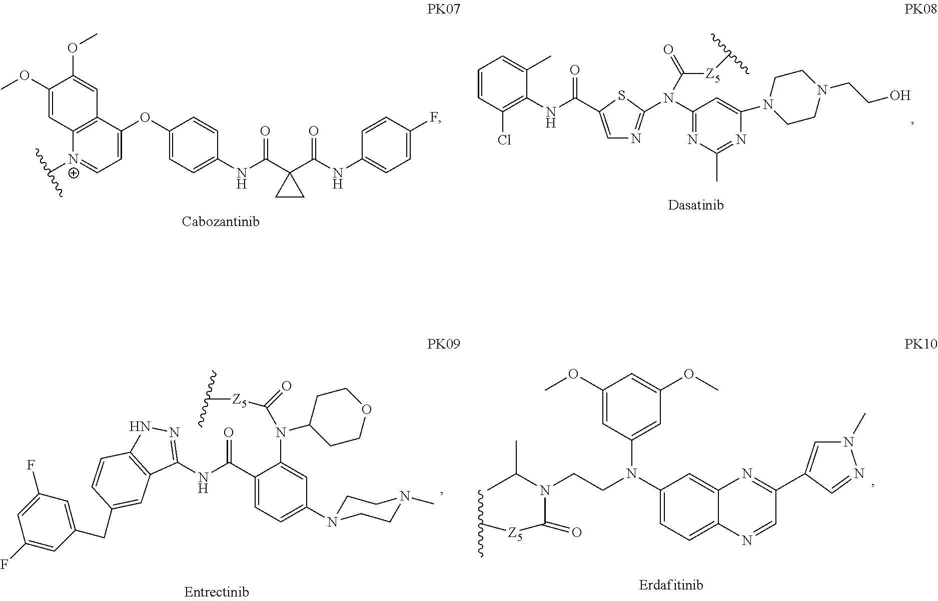

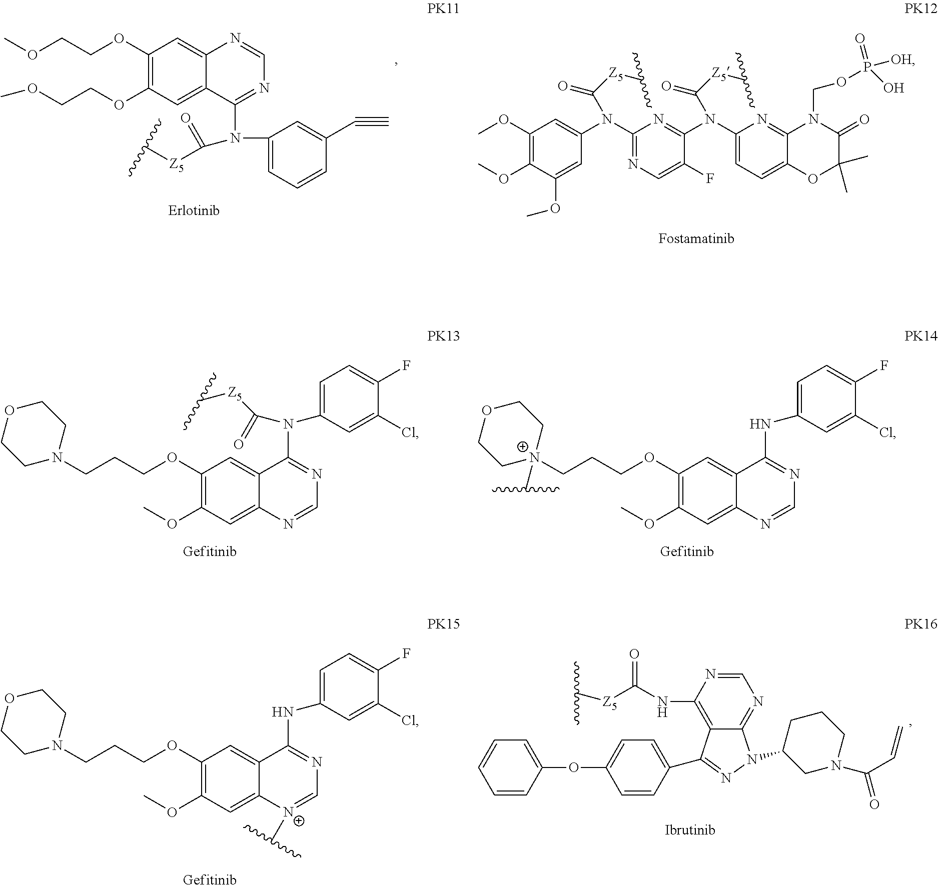

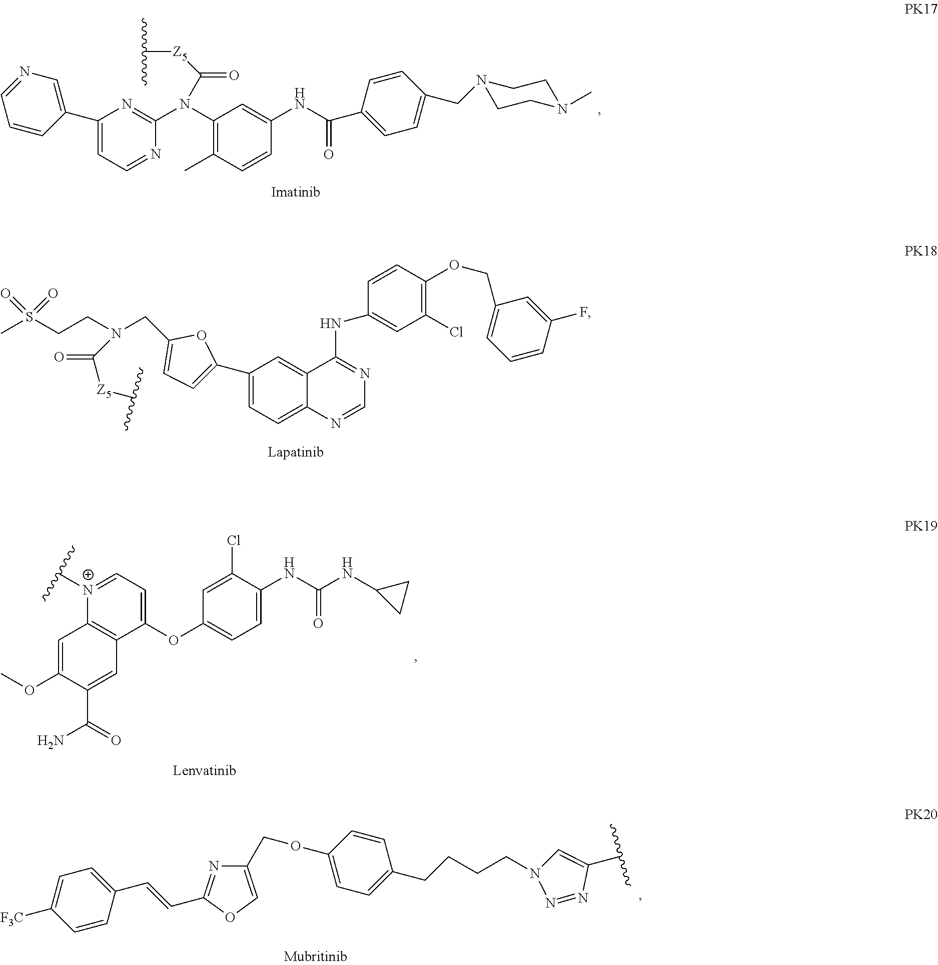

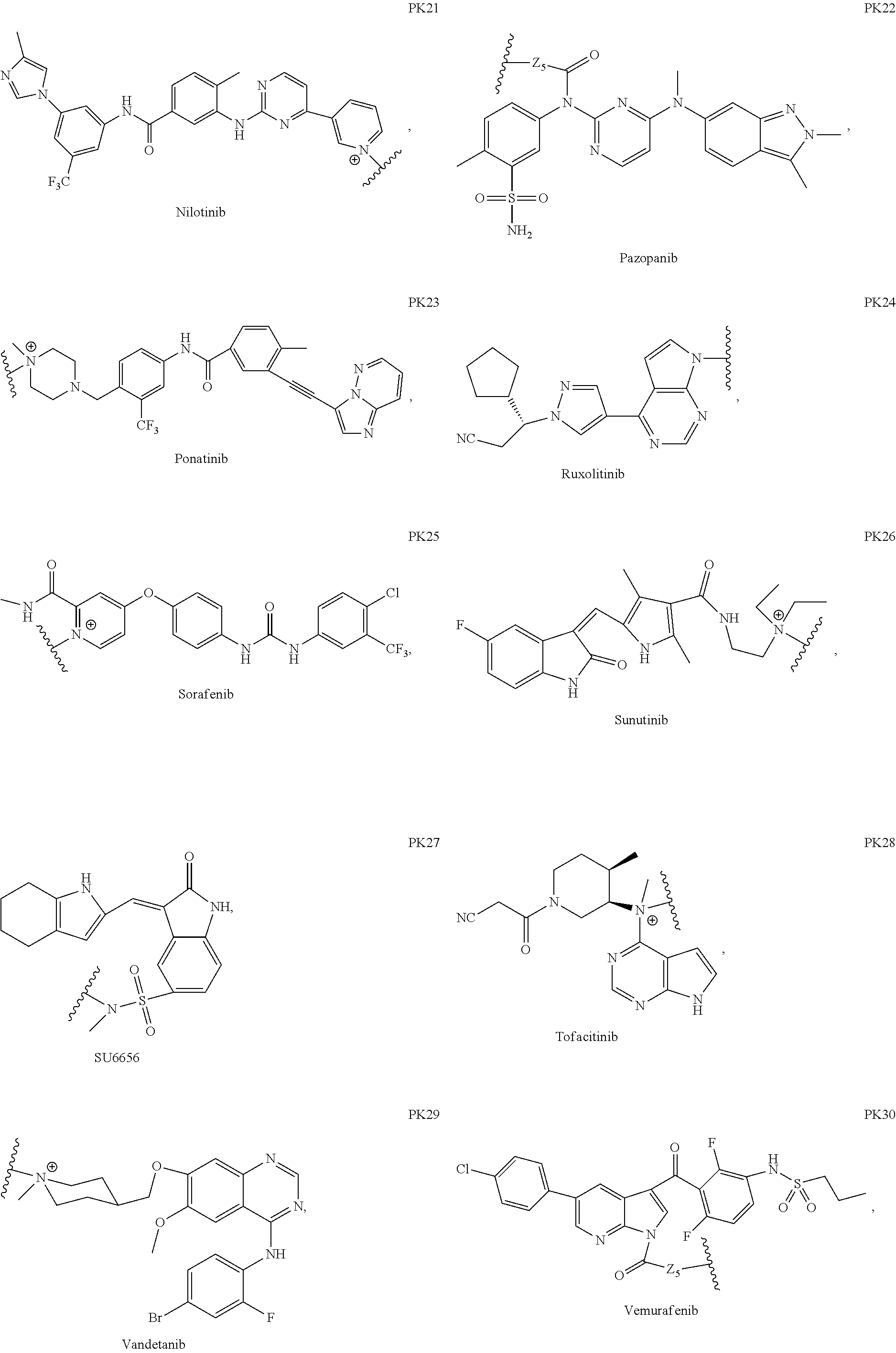

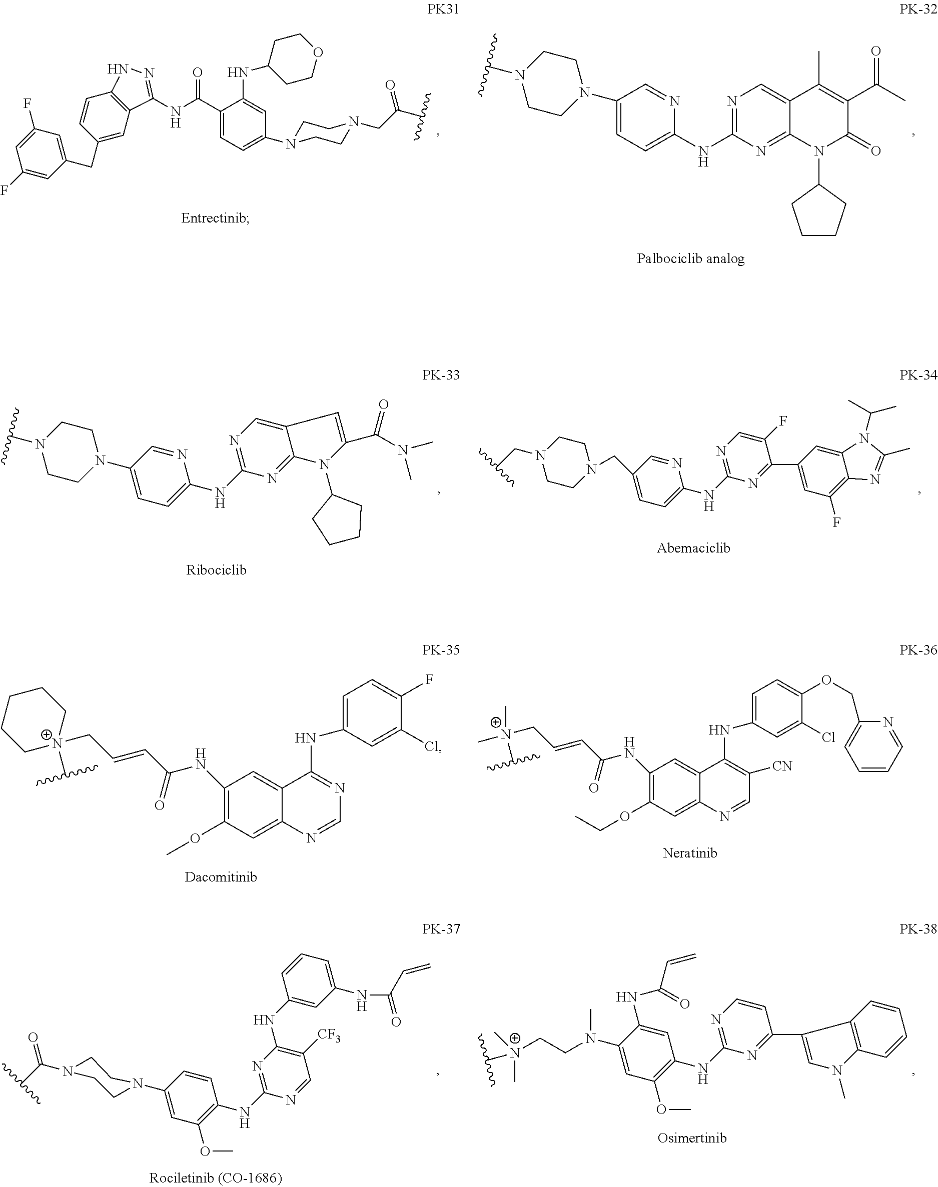

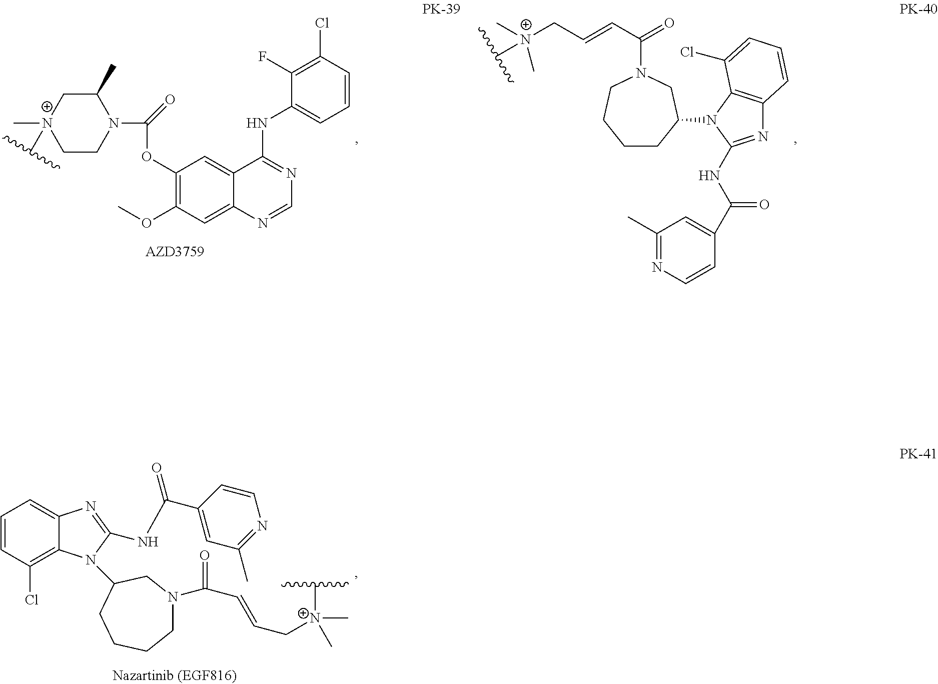 [SMILES: *[N+]1(C)CCN(C(=O)Oc2cc3c(Nc4cccc(Cl)c4F)ncnc3cc2OC)[C@H](C)C1 ; *[N+](C)(C)C/C=C/C(=O)N1CCCC[C@@H](n2c(NC(=O)c3ccnc(C)c3)nc3cccc(Cl)c32)C1 ; *[N+](C)(C)C/C=C/C(=O)N1CCCCC(n2c(NC(=O)c3ccnc(C)c3)nc3cccc(Cl)c32)C1]

or hydrated salts; or the polymorphic crystalline structures of these compounds; or the optical isomers, racemates, diastereomers or enantiomers;
wherein Zs and Zs' are independently selected from O, NH, NHNH, NR₅, S, C(O)O, C(O)NH, OC(O)NH, OC(O)O, NHC(O)O, NHC(O)NH, NHC(O) S, OC(O)N(R₁), N(R₁) C(O)N(R₂), C(O)NHNHC(O) and C(O)NR₁.

In certain such embodiments, proteinase inhibitors are also preferred as cytotoxic agents of the present patent. The proteinase inhibitors are selected from Carfilzomib, Clindamycin, Retapamulin, Indibulin, having the following formulae:

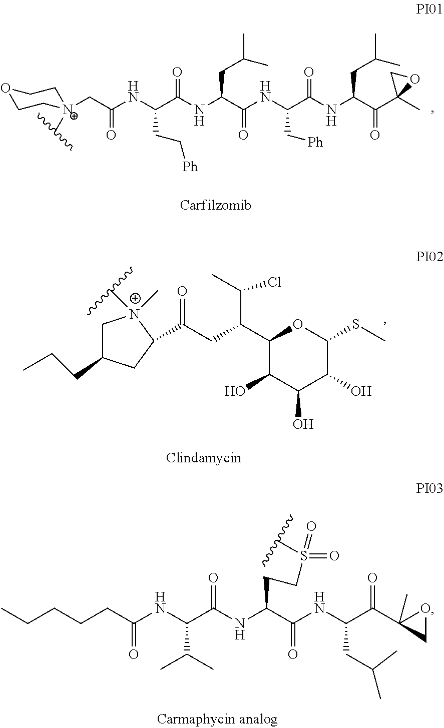

or an isotope of one or more chemical elements, or pharmaceutically acceptable salts, hydrates, or hydrated salts; or the polymorphic crystalline structures of these compounds; or the optical isomers, racemates, diastereomers or enantiomers;
[symbol] is a linkage site that links to L₁ and/or L₂ independently.

In certain such embodiments, an immunotoxin is also preferred as cytotoxic agents of the present patent. The immunotoxin is selected from Diphtheria toxin (DT), Cholera toxin (CT), Trichosanthin (TCS), Dianthin, Pseudomonas exotoxin A (ETA), Erythrogenic toxins, Diphtheria toxin, AB toxins, Type III exotoxins, proaerolysin, and topsalysin;

In certain such embodiments, an immunotoxin herein can be a cytotoxic agent of the invention. The immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves; An immunotoxin herein is preferably conjugated via the process of the application through an amino acid having free amino, thiol or carboxyl acid group; and more preferably through N-terminal amino acid.

In addition, a certain cell receptor agonist, a cell stimulating molecule or intracellular signalling molecule can be as a chemotherapeutic/cytotoxic agent conjugated to the antibody of the invention.

In certain embodiments, one, two or more DNA, RNA, mRNA, small interfering RNA (siRNA), microRNA (miRNA), and PIWI interacting RNAs (piRNA) can be as a chemotherapeutic/function compound conjugated to the antibody of the invention:

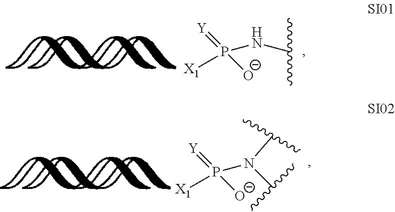

wherein “ [symbol] ” is the site to link the side chain linker of the present patent; [symbol] is single or double strands of DNA, RNA, mRNA, siRNA, miRNA, or piRNA; X₁, and Y are independently O, NH, NHNH, NR₁, S, C(O)O, C(O)NH, OC(O)NH, OC(O)O, NHC(O)NH, NHC(O) S, OC(O)N(R₁), N(R₁) C(O)N(R₁), CH₂, C(O)NHNHC(O) and C(O)NR₁.

In certain such embodiments, a MEK inhibitor can be the cytotoxic agent of the invention. A MEK inhibitor inhibits the mitogen-activated protein kinases MEK1 and/or MEK2 which is often overactive in some cancers. MEK inhibitors are especially used for treatment of BRAF-mutated melanoma, and KRAS/BRAF mutated colorectal cancer, breast cancer, and non-small cell lung cancer (NSCLC). MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733. The preferred MEK inhibitors are selected from Trametinib (GSK1120212), Cobimetinib (XL518), Binimetinib (MEK162), selumetinib having the following formula:

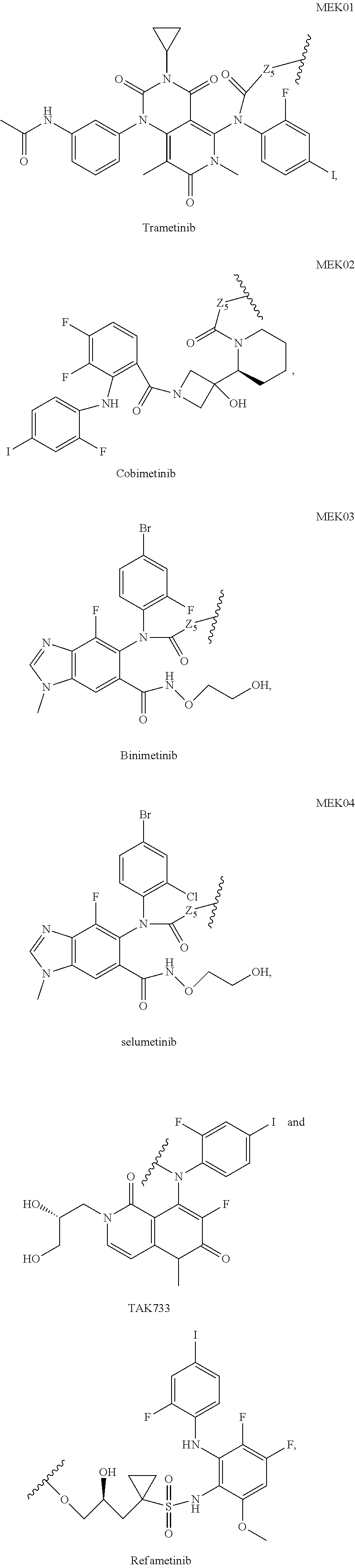

or an isotope of one or more chemical elements, or pharmaceutically acceptable salts, hydrates, or hydrated salts; or the polymorphic crystalline structures of these compounds; or the optical isomers, racemates, diastereomers or enantiomers; [symbol] is a linkage site;
wherein Zs is selected from O, NH, NHNH, NR₅, S, C(O)O, C(O)NH, OC(O)NH, OC(O)O, NHC(O)O, NHC(O)NH, NHC(O) S, OC(O)N(R₁), N(R₁) C(O)N(R₂), C(O)NHNHC(O) and C(O)NR₁;

In a further embodiment, either one or both of Drug 1 or/and Drug 2 of the present patent application can be independently selected from a chemotherapeutical drug as following:
1). Poly (ADP-ribose) polymerase inhibitors: also are called PARP inhibitors. Poly (ADP-ribose) polymerases (PARPs) are a family of related enzymes that share the ability to catalyze the transfer of ADP-ribose to target proteins. PARPs play an important role in various cellular processes, including modulation of chromatin structure, transcription, replication, recombination, and DNA repair. PARP inhibitors disrupt DNA repair mechanisms by inhibiting PARP enzymatic activity, leading to the accumulation of DNA damage and cell death. Examples of PARP inhibitors include but not limited are Olaparib, Niraparib, Rucaparib, Veliparib, Talazoparib, Pamiparib, Fluzoparib, 5F02, Simmiparib, DDHCB, BTH-8, YHP-836, ZC-22, Mefuparib, 1,2,4-triazoles, Mortaparib, 2X-121, NMS-03305293, AZD5305, RP12146, AZD9574, E7449, AMXI-5001, IMP4297, FluorThanatrace. The conjugatable structures (containing a linkage site) of the PARP inhibitors are illustrated below:

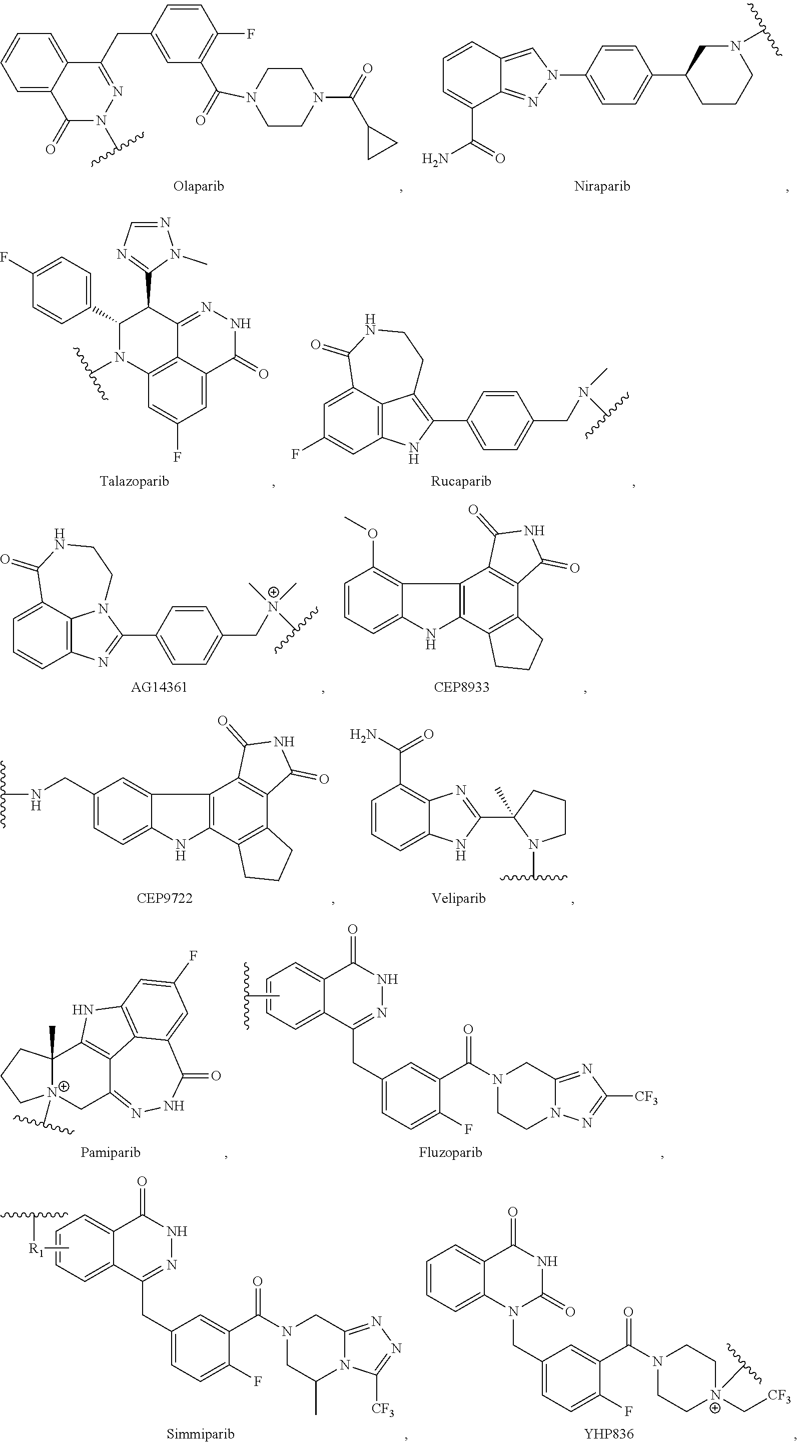

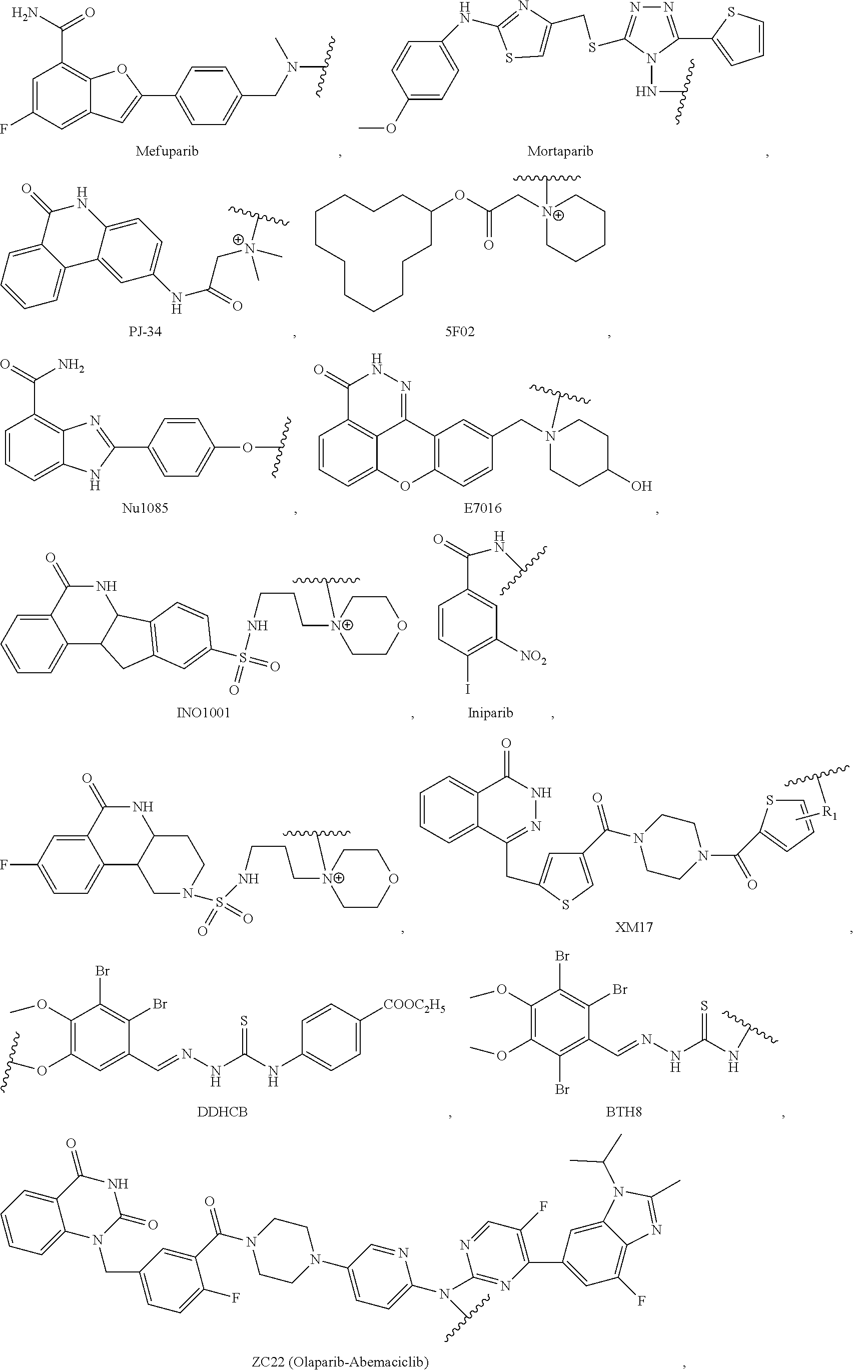

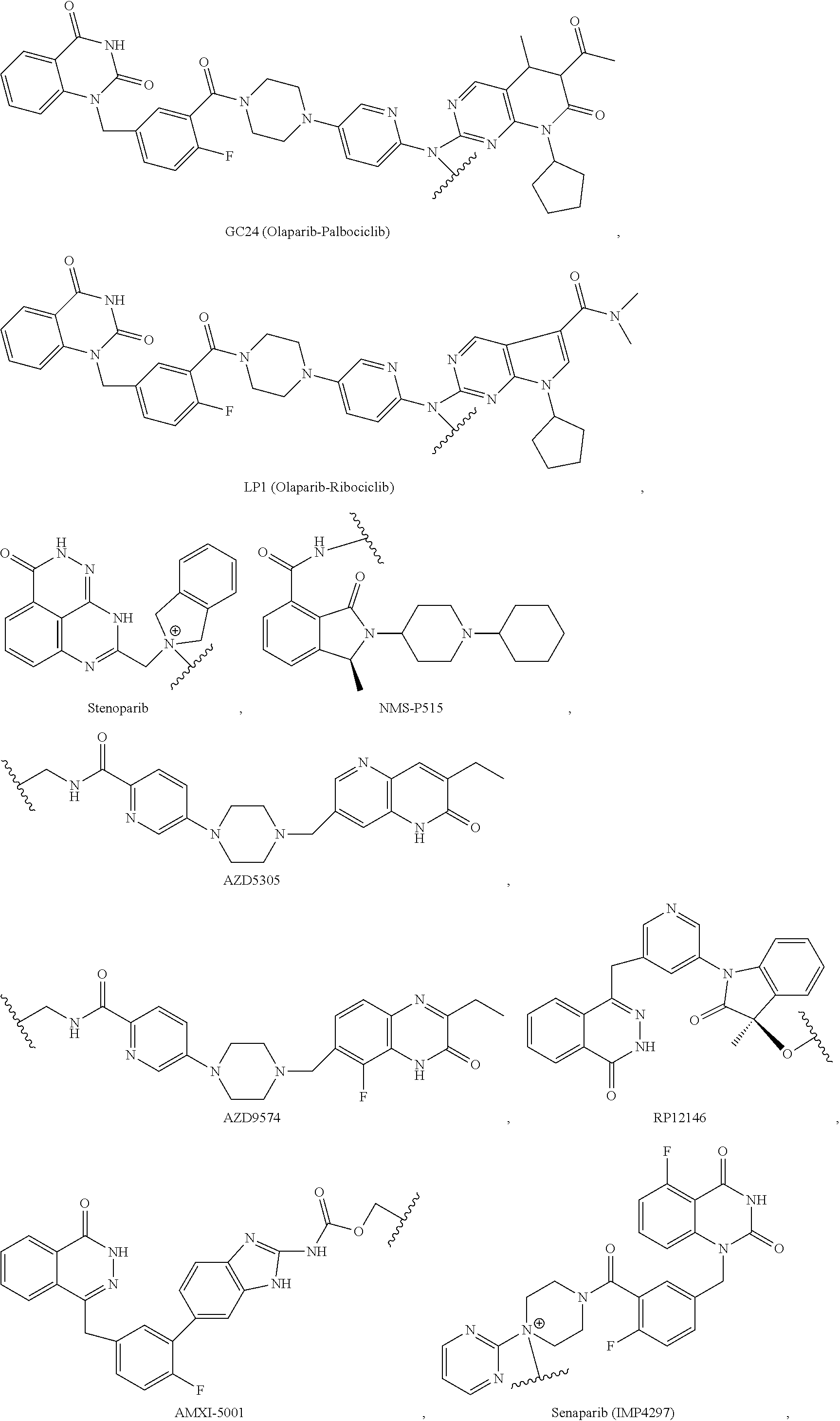

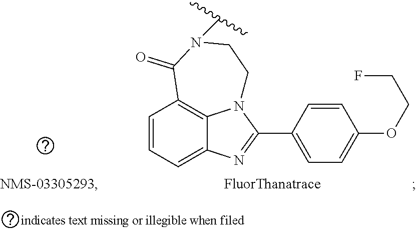

wherein is [symbol] the linkage site.
2). Inhibitors of Bromodomain-containing proteins, in particular BRD4: The bromodomain acts to recognize acetylated lysine in histones and transcription proteins and plays a fundamental role in chromatin-based cellular processes including gene transcription and chromatin remodeling. The bromodomain and extra terminal domain (BET) protein have been implicated in cancers and inflammatory disorders and recognized as attractive drug targets. The BET family consists of four proteins: bromodomain containing protein 2 (BRD2), BRD3, BRD4, and bromodomain testis-specific protein (BRDT). Each of these contains two tandem BDs, known as BD1 (N-terminus) and BD2 (C-terminus), with high similarity across family members. BRD4 bromodomain protein has developed to be an interesting drug target for the treatment of cancer [E. Nicodeme, et al, Nature 468 (2010) 1119-1123. J. Zuber, et al, Nature 478 (2011) 524-528.], obesity [A. C. Belkina, et al, Nat. Rev. Cancer 12 (2012) 465-477.], kidney disease [G. Zhang, et al, J Biol Chem 287 (2012) 28840-28851.], lung fibrosis [X. Tang, et al, Am. J. Pathol. 183 (2013) 470-479.] and other inflammatory diseases [B. Huang, et al, Mol. Cell Biol. 29 (2009) 1375-1387]. Many BET inhibitors have been developed to prevent the recognition of interaction between BDs and the acetylated histones. Among them, the compounds OTX-015, ABBV-075, and INCB057643 have been advanced into human clinical trials. Some of the preferred conjugatable structures of the inhibitors of the bromodomain and extra terminal domain (BET) proteins of the invention are illustrated below:

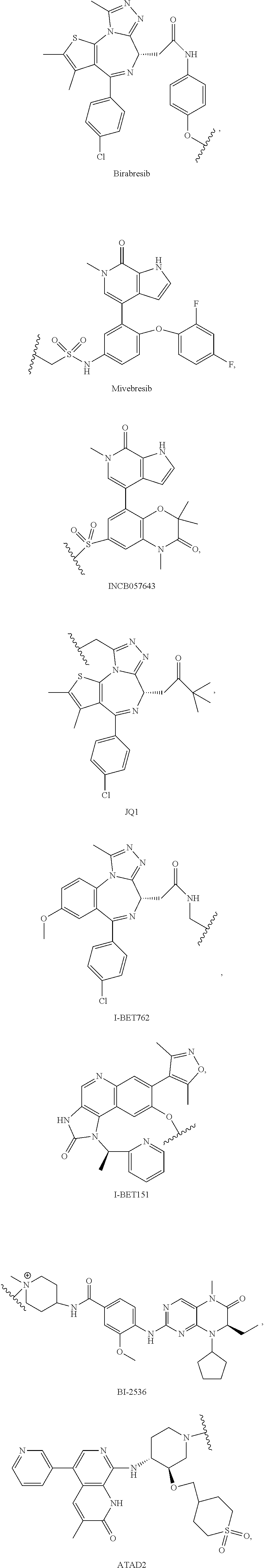

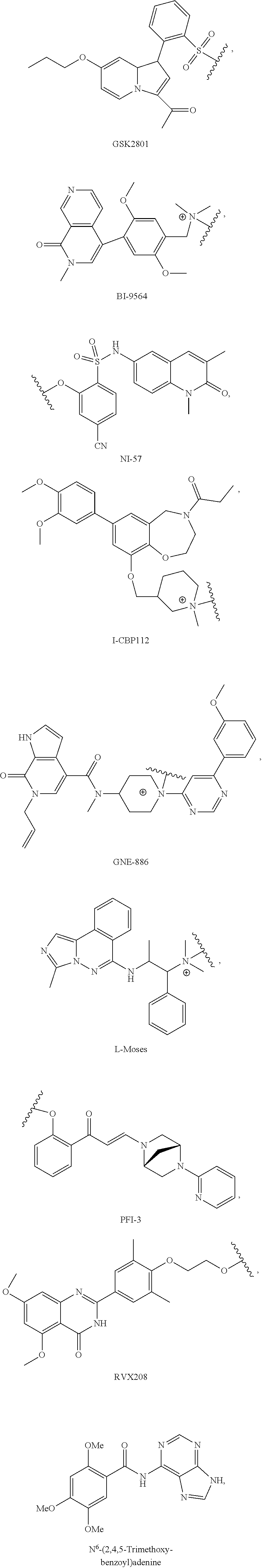

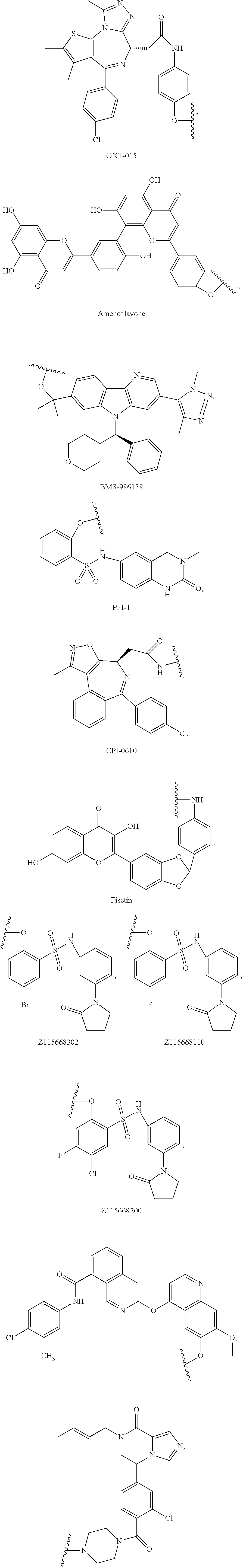

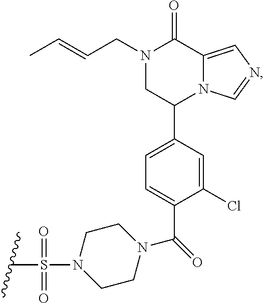 [SMILES: *S(=O)(=O)N1CCN(C(=O)c2ccc(C3CN(C/C=C/C)C(=O)c4cncn43)cc2Cl)CC1]

or hydrated salts; or the polymorphic crystalline structures of these compounds; or the optical isomers, racemates, diastereomers or enantiomers; wherein [symbol] is the linkage site.
3). An inhibitor of Cyclin-dependent kinases (CDKs): Cyclin-dependent kinases (CDKs) are serine/threonine kinases responsible for the coordination of critical regulatory events during cell cycle and transcription. Their catalytic activities are modulated by interactions with cyclins and CDK inhibitors (CKIs). Thus, it is anticipated that deregulation of CDKs is a common feature of many cancers. Early pan-CDK inhibitors showed poor results in clinical trials for solid malignancies, as the lack of selectivity produced significant toxicity. The production of more selective inhibitors led to significant developments in cancer therapy, as CDK4/CDK6 inhibitors in combination with endocrine therapy changed the landscape of the treatment of hormone-receptor positive (HR+) metastatic breast cancer (MBC) and other tumor types as well. The extensive studies of cyclin-dependent kinase inhibitors, include but not limited, palbociclib (PD-0332991, Ibrance®, CDK4/6 inhibitor), ribociclib (LEE-011, Kisqali®, CDK4/6 inhibitor), abemaciclib (LY2835219, Verzenio™, CDK4/6 inhibitor), PF-06873600 (CDK2/4/6 inhibitor), GIT28 (trilaciclib, CDK4/6 inhibitor), GIT38 (Lerociclib, CDK4/6 inhibitor), SHR-6390 (CDK4/6 inhibitor), PHA848125 (Milciclib, CDK2 inhibitor), FN-1501 (CDK2/4/6 inhibitor), AGM-130 (Inditinib, CDK1/2/4/5/6 inhibitor), (−)—BPI-16350 (CDK4/6 inhibitor), Flavopiridol (Alvocidib, CDK9/1/4/5/7 inhibitor), PHA848125 (Milciclib, CDK2/1/4/5/7 and TrKA inhibitor), BCD115 (CDK8/19 inhibitor), MM-D₃₇K (CDK4/6 inhibitor), PP-06873600 (CDK2/4/6 inhibitor), TG02 (SB-1317, Zotiraciclib, (CDK9/1/2/3/5/7, FLT3, JAK2 and MAPK7 inhibitor), C₇₀₀₁ (ICEC0942, CDK7 inhibitor), BEY1107 (CDK1 inhibitor), XZP3287 (Birociclib, CDK4/6 inhibitor), BPI16350 (CDK4/6 inhibitor), FCN437 (CDK4/6 inhibitor), CYC065 (CDK2/9/5 inhibitor), Seliciclib (CY202, R-Roscovitine, CDK2/9/1/5/7 inhibitor), AT7519 (CDK9/1/2/3/5/4/6, GSK36 and VEGFR inhibitor), AGM130 (Inditinib, CDK2/1/4/5/6 inhibitor), FN1501 (CDK2/4/6 and FLT3 inhibitor), SY1365 (CDK7/12 inhibitor), AZD4573 (CDK9 inhibitor), TP1287 (Flavopiridol prodrug, CDK9 inhibitor), P1446A-05 (Voruciclib, CDK9/1/4 inhibitor), BAY1251152 (CDK9 inhibitor), SCH727965 (MK7965, Dinaciclib, (CDK12/1/2/5/9 inhibitor), BEBT209 (CDK4/6 inhibitor), TQB3616 (CDK4/6 inhibitor), AT7519, BAY1000394 (Roniciclib, CDK1/2/3/4/7/9 and VEGFR inhibitor), BAY1143572 (Atuveciclib, CDK9 inhibitor), Bay 117082, THZ531 (CDK12 inhibitor), AMG925 (FLX925, CDK4/6 and FLT3 inhibitor). The conjugatable structures of some above compounds are illustrated below:

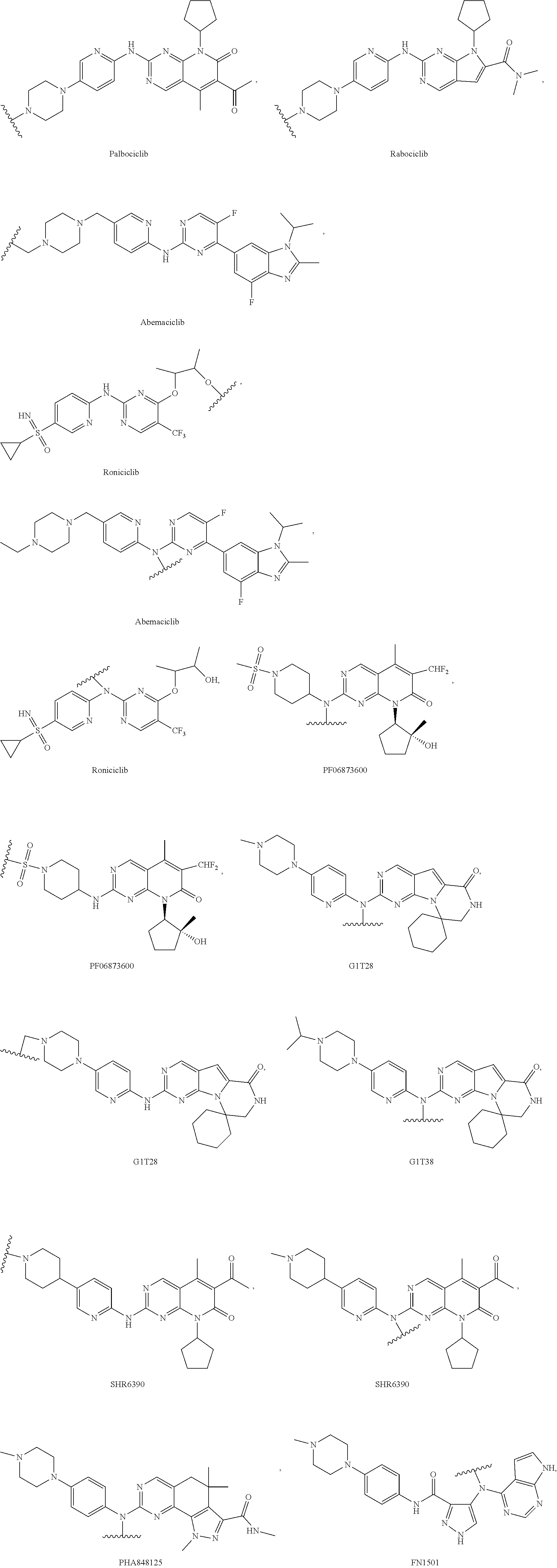

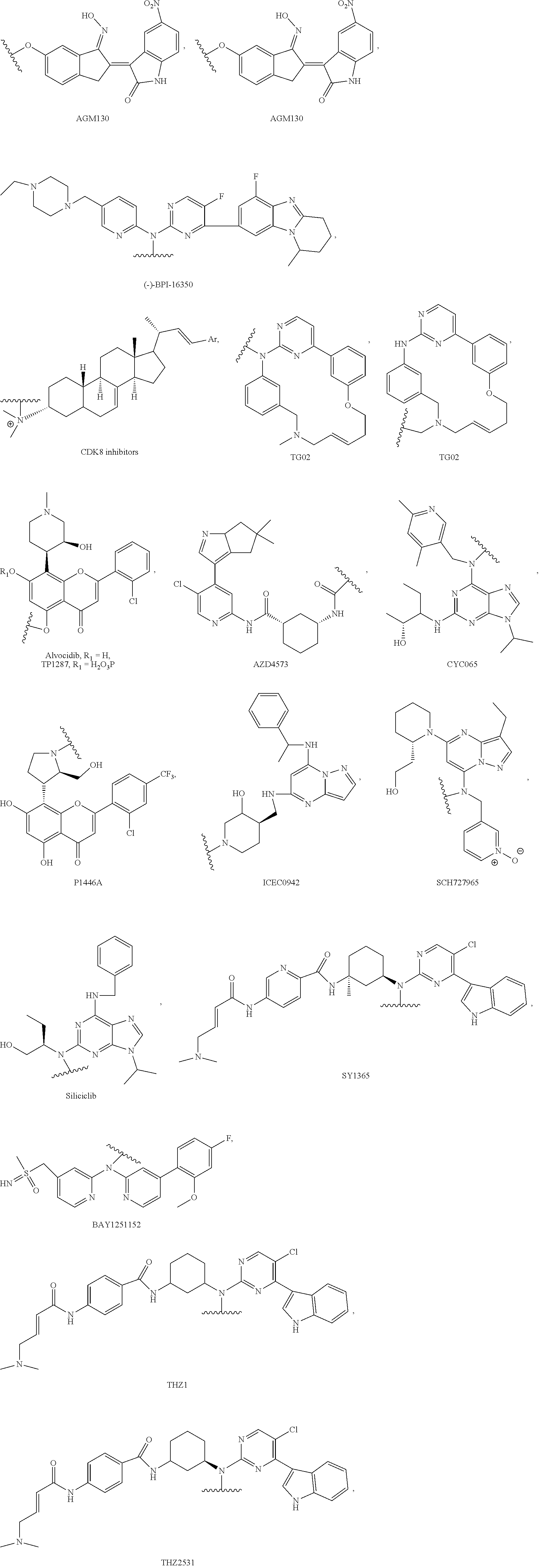

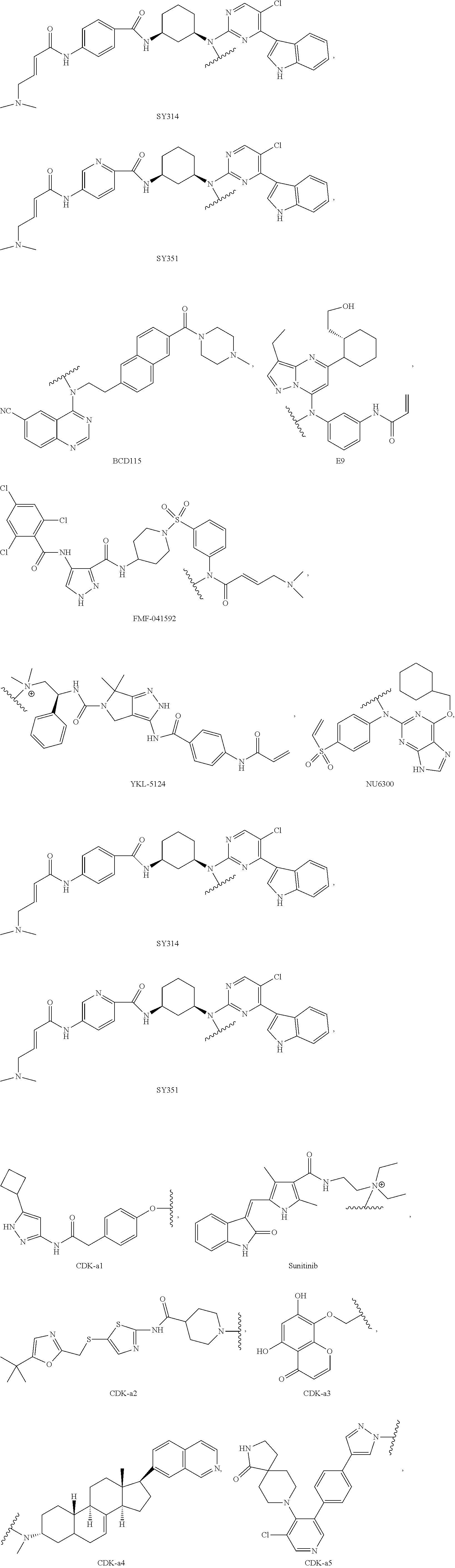

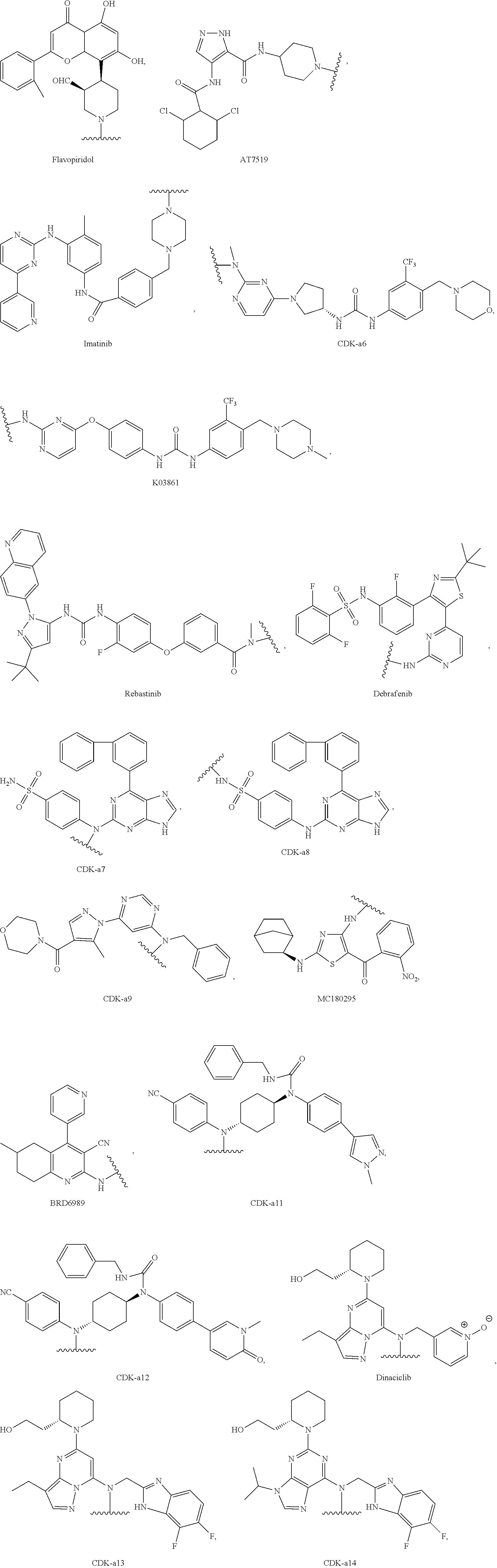

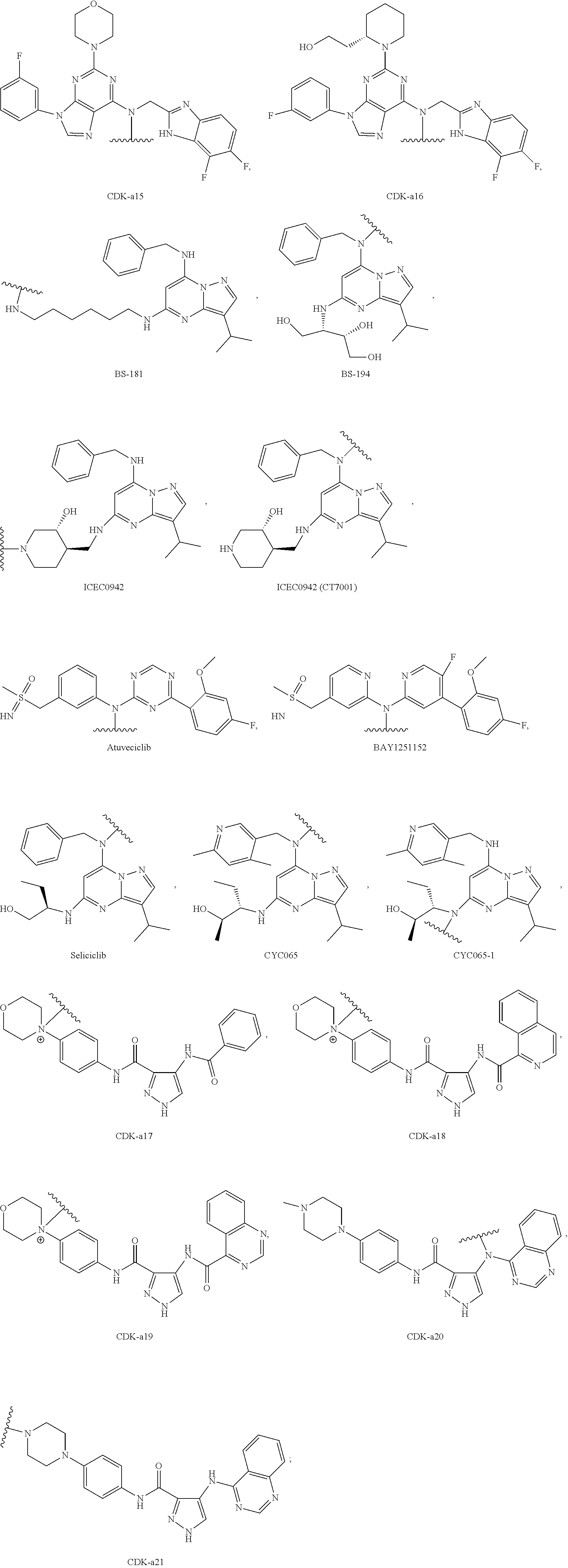

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the linkage site.
4). An EGFR inhibitor: Epidermal growth factor receptor (EGFR, also known as ErbB-1 or HER-1) inhibitors are medicines that bind to certain parts of the EGFR and slow down or stop cell growth. EGFR is a protein that is found on the surface of some cells that causes cells to divide when epidermal growth factor binds to it. EGFR is found at abnormally high levels in cancer cells, and EGFR activation appears to be important in tumor growth and progression. Some types of cancers show mutations in their EGFRs, which may cause unregulated cell division through continual or abnormal activation of the EGFR. EGFR inhibitors can normally be classified as tyrosine kinase inhibitors (TKI) which bind to the tyrosine kinase domain in the epidermal growth factor receptor and stop the activity of the EGFR. EGFR inhibitors may be used in the treatment of cancers that are caused by EGFR up-regulation, such as non-small-cell lung cancer, pancreatic cancer, breast cancer, and colon cancer. Tyrosine kinase inhibitors (TKIs), mostly small molecules and monoclonal antibodies that have been approved for clinical use or at least have entered clinical trials, are classified according to their main target: HER, IGF-1R, HGFR, VEGF, BCR-ABL, ALK, JAK, BTK, and TRK. Some of the conjugatable structures of the EGFR inhibitors/TKIs are illustrated below:

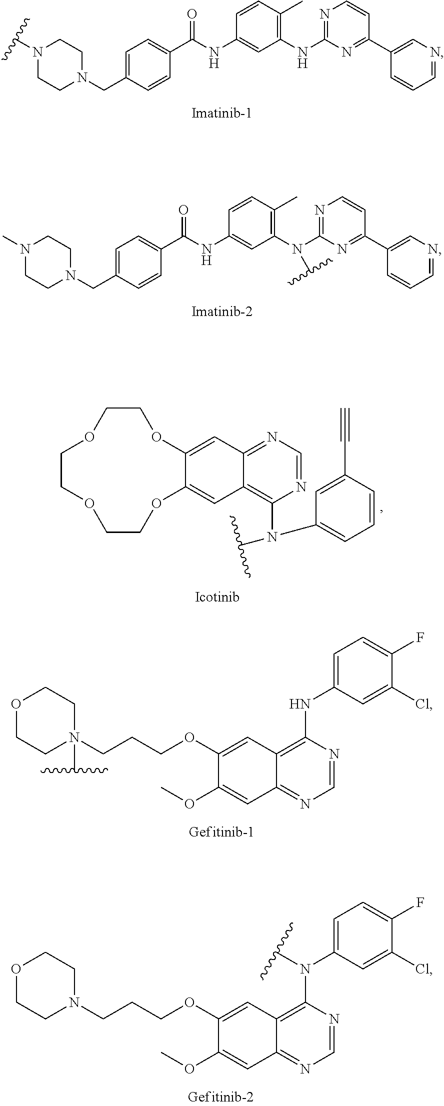

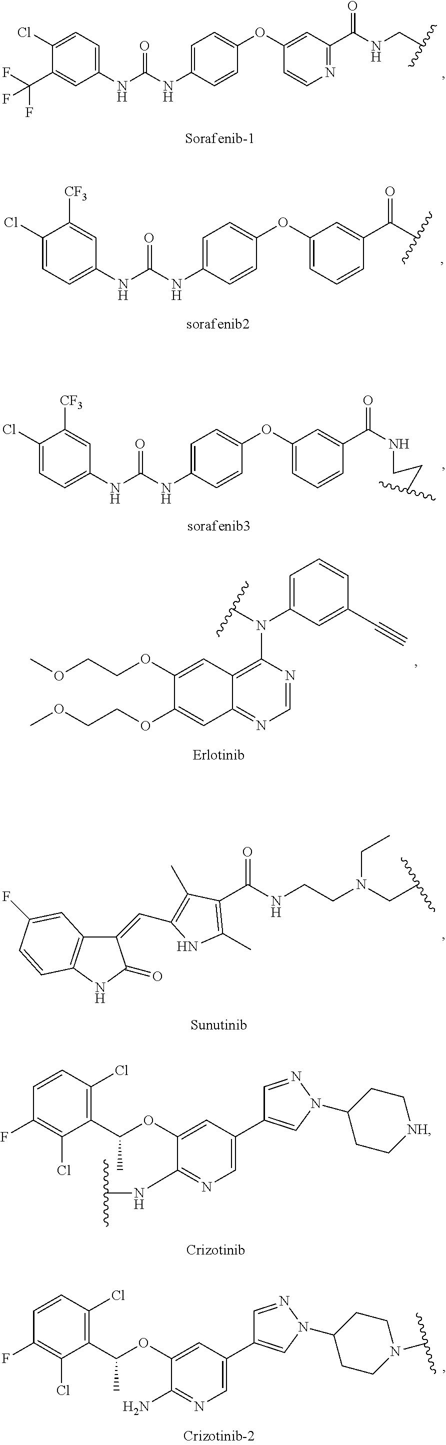

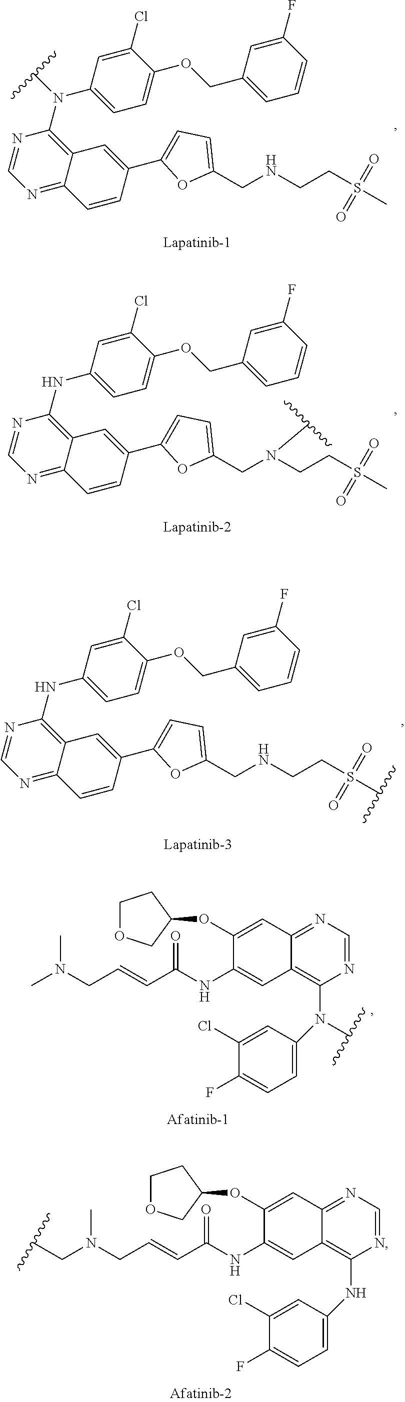

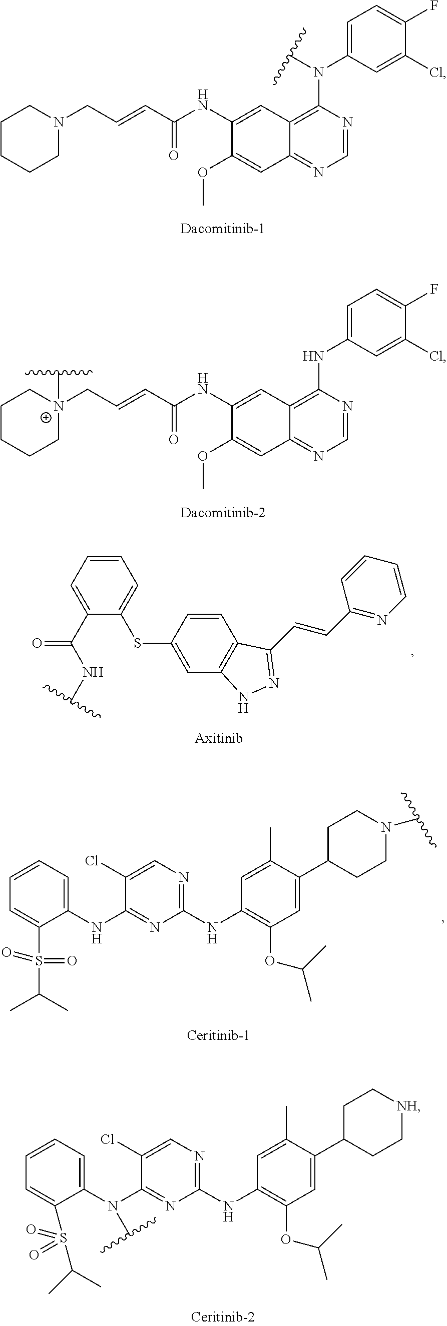 [SMILES: *N(c1ccc(F)c(Cl)c1)c1ncnc2cc(OC)c(NC(=O)/C=C/CN3CCCCC3)cc12 ; *[N+]1(C/C=C/C(=O)Nc2cc3c(Nc4ccc(F)c(Cl)c4)ncnc3cc2OC)CCCCC1 ; *NC(=O)c1ccccc1Sc1ccc2c(/C=C/c3ccccn3)n[nH]c2c1 ; *N1CCC(c2cc(OC(C)C)c(Nc3ncc(Cl)c(Nc4ccccc4S(=O)(=O)C(C)C)n3)cc2C)CC1 ; *N(c1ccccc1S(=O)(=O)C(C)C)c1nc(Nc2cc(C)c(C3CCNCC3)cc2OC(C)C)ncc1Cl]

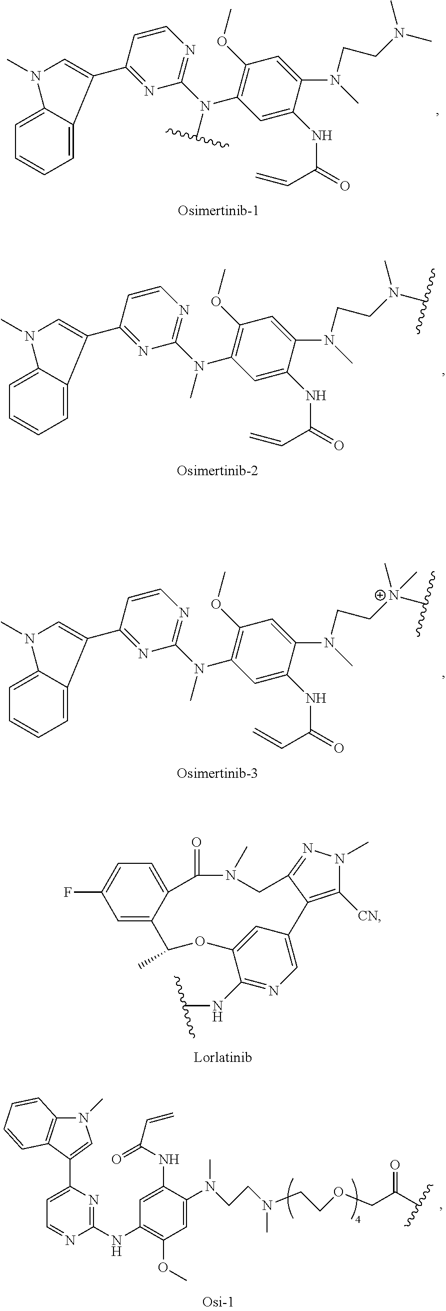

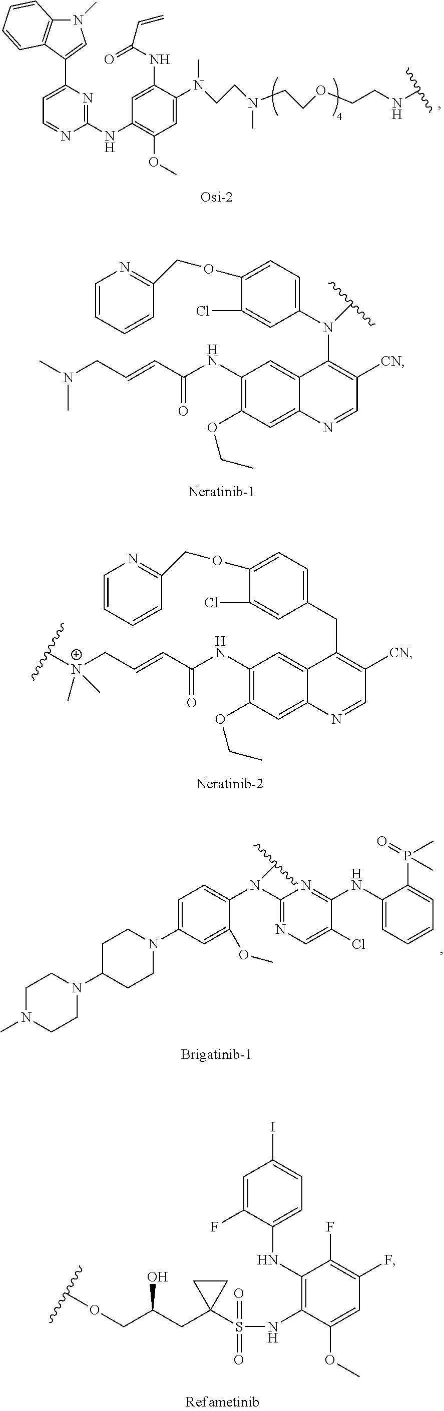

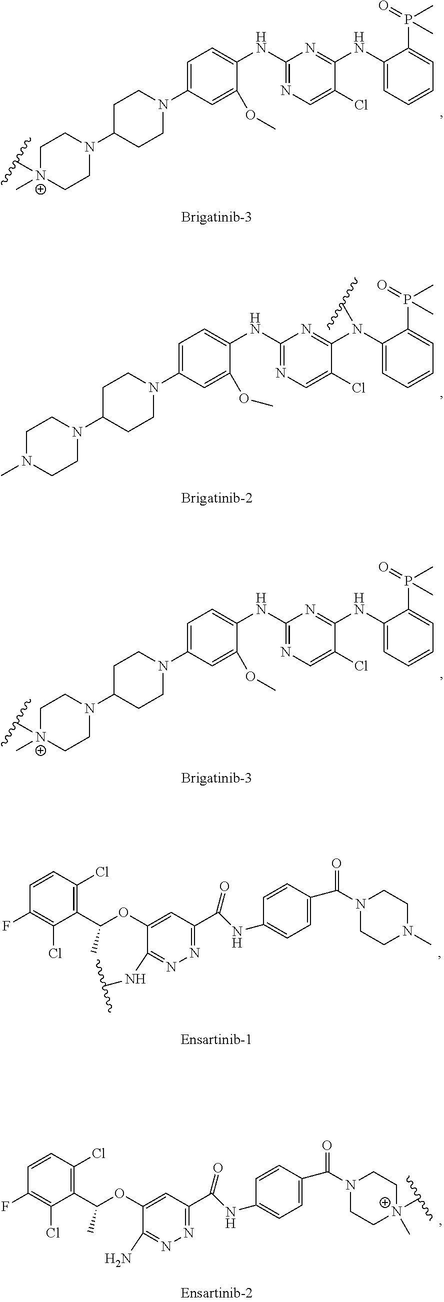

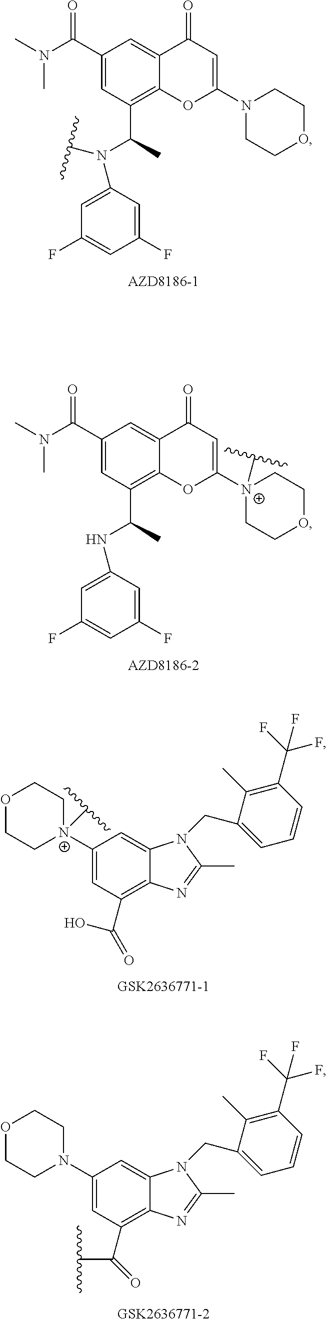 [SMILES: *N(c1cc(F)cc(F)c1)[C@H](C)c1cc(C(=O)N(C)C)cc2c(=O)cc(N3CCOCC3)oc12 ; *[N+]1(c2cc(=O)c3cc(C(=O)N(C)C)cc([C@@H](C)Nc4cc(F)cc(F)c4)c3o2)CCOCC1 ; *[N+]1(c2cc(C(=O)O)c3nc(C)n(Cc4cccc(C(F)(F)F)c4C)c3c2)CCOCC1 ; *C(=O)c1cc(N2CCOCC2)cc2c1nc(C)n2Cc1cccc(C(F)(F)F)c1C]

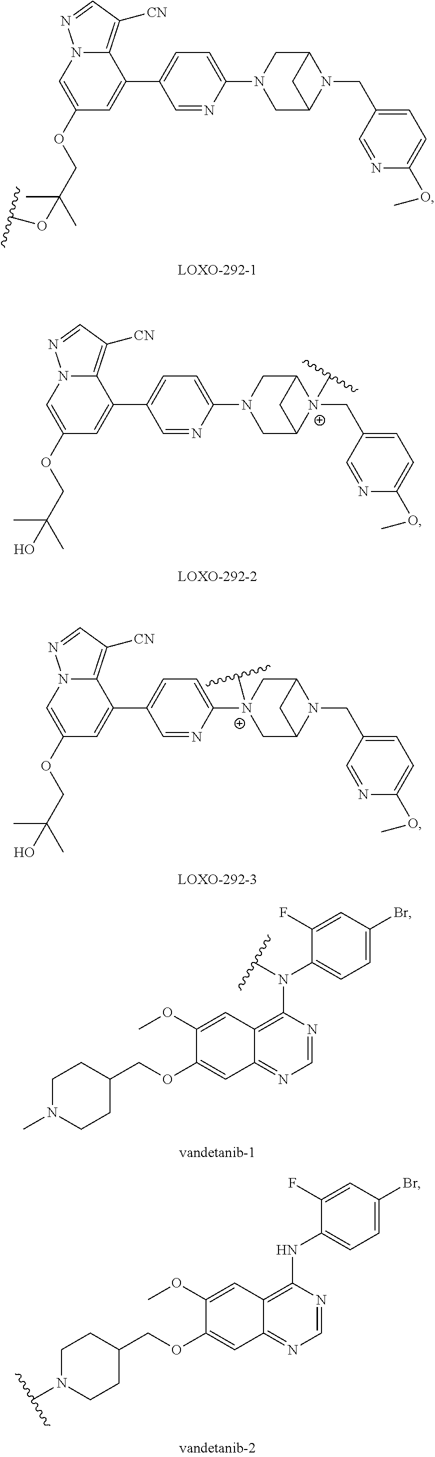 [SMILES: *OC(C)(C)COc1cc(-c2ccc(N3CC4CC(C3)N4Cc3ccc(OC)nc3)nc2)c2c(C#N)cnn2c1 ; *[N+]1(Cc2ccc(OC)nc2)C2CC1CN(c1ccc(-c3cc(OCC(C)(C)O)cn4ncc(C#N)c34)cn1)C2 ; *[N+]1(c2ccc(-c3cc(OCC(C)(C)O)cn4ncc(C#N)c34)cn2)CC2CC(C1)N2Cc1ccc(OC)nc1 ; *N(c1ccc(Br)cc1F)c1ncnc2cc(OCC3CCN(C)CC3)c(OC)cc12 ; *N1CCC(COc2cc3ncnc(Nc4ccc(Br)cc4F)c3cc2OC)CC1]

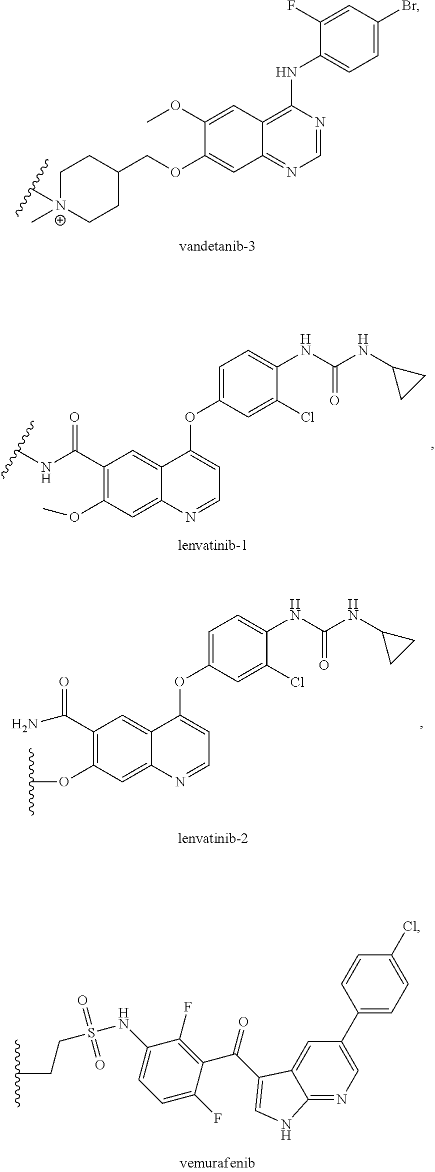 [SMILES: *[N+]1(C)CCC(COc2cc3ncnc(Nc4ccc(Br)cc4F)c3cc2OC)CC1 ; *NC(=O)c1cc2c(Oc3ccc(NC(=O)NC4CC4)c(Cl)c3)ccnc2cc1OC ; *Oc1cc2nccc(Oc3ccc(NC(=O)NC4CC4)c(Cl)c3)c2cc1C(N)=O ; *CCS(=O)(=O)Nc1ccc(F)c(C(=O)c2c[nH]c3ncc(-c4ccc(Cl)cc4)cc23)c1F]

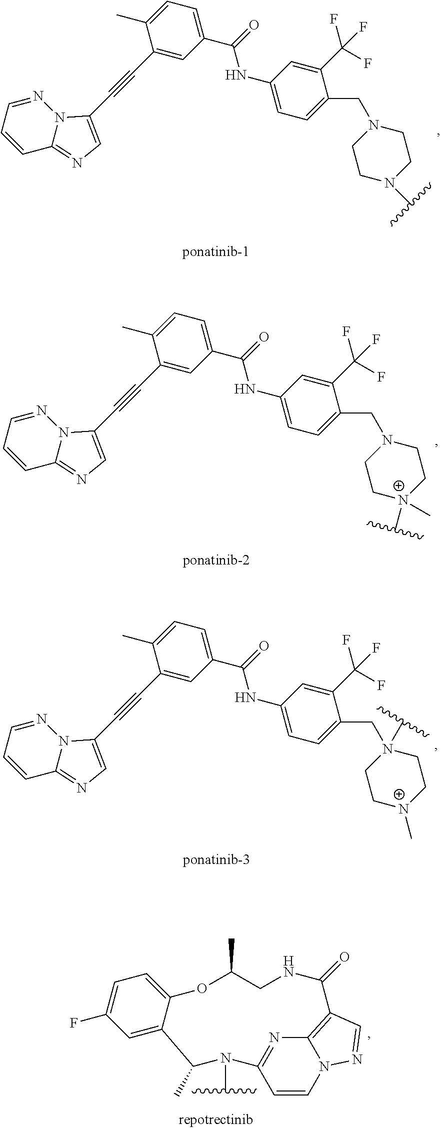

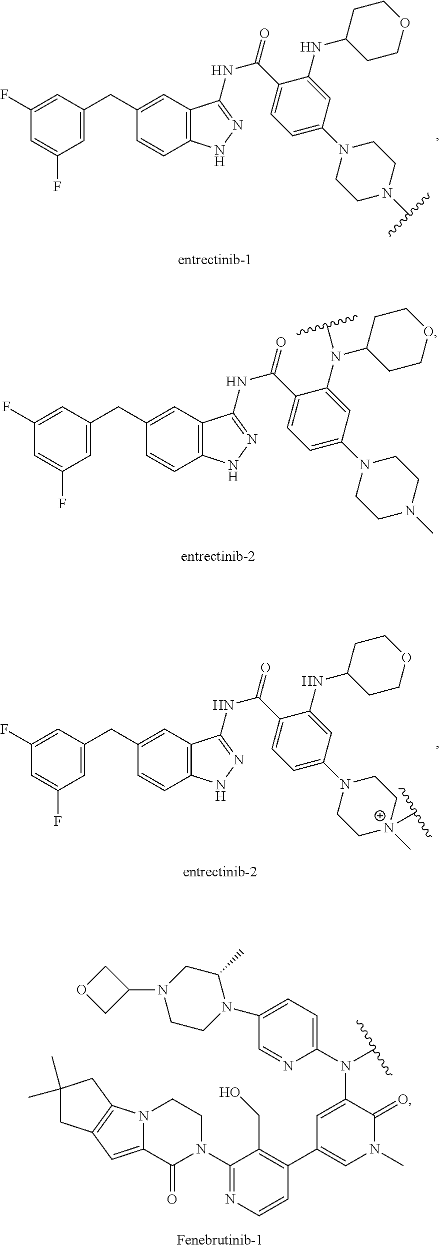

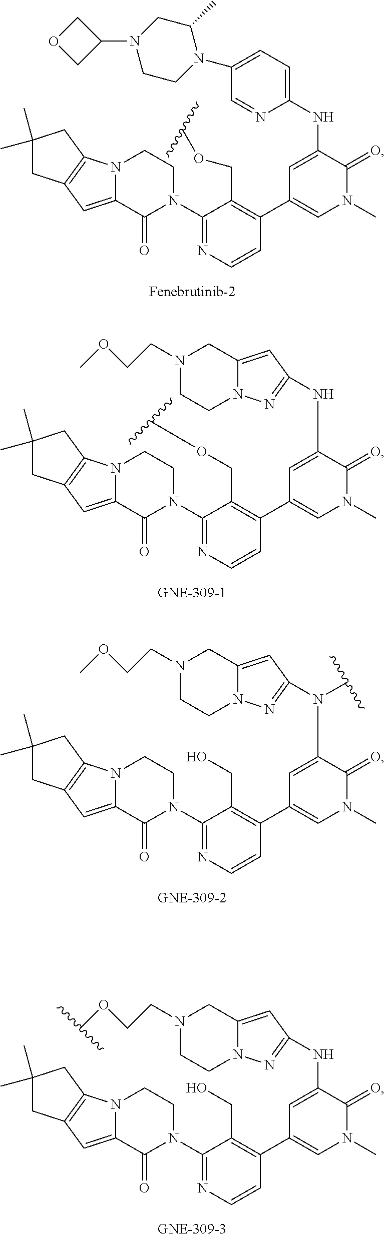 [SMILES: *OCc1c(-c2cc(Nc3ccc(N4CCN(C5COC5)C[C@@H]4C)cn3)c(=O)n(C)c2)ccnc1N1CCn2c(cc3c2CC(C)(C)C3)C1=O ; *OCc1c(-c2cc(Nc3cc4n(n3)CCN(CCOC)C4)c(=O)n(C)c2)ccnc1N1CCn2c(cc3c2CC(C)(C)C3)C1=O ; *N(c1cc2n(n1)CCN(CCOC)C2)c1cc(-c2ccnc(N3CCn4c(cc5c4CC(C)(C)C5)C3=O)c2CO)cn(C)c1=O ; *OCCN1CCn2nc(Nc3cc(-c4ccnc(N5CCn6c(cc7c6CC(C)(C)C7)C5=O)c4CO)cn(C)c3=O)cc2C1]

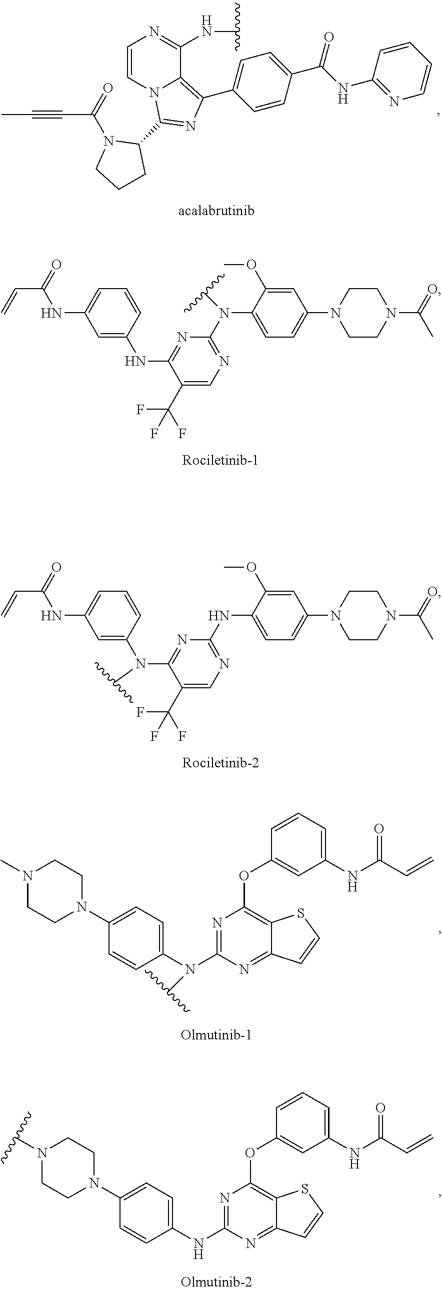 [SMILES: *Nc1nccn2c([C@@H]3CCCN3C(=O)C#CC)nc(-c3ccc(C(=O)Nc4ccccn4)cc3)c12 ; *N(c1ncc(C(F)(F)F)c(Nc2cccc(NC(=O)C=C)c2)n1)c1ccc(N2CCN(C(C)=O)CC2)cc1OC ; *N(c1cccc(NC(=O)C=C)c1)c1nc(Nc2ccc(N3CCN(C(C)=O)CC3)cc2OC)ncc1C(F)(F)F ; *N(c1ccc(N2CCN(C)CC2)cc1)c1nc(Oc2cccc(NC(=O)C=C)c2)c2sccc2n1 ; *N1CCN(c2ccc(Nc3nc(Oc4cccc(NC(=O)C=C)c4)c4sccc4n3)cc2)CC1]

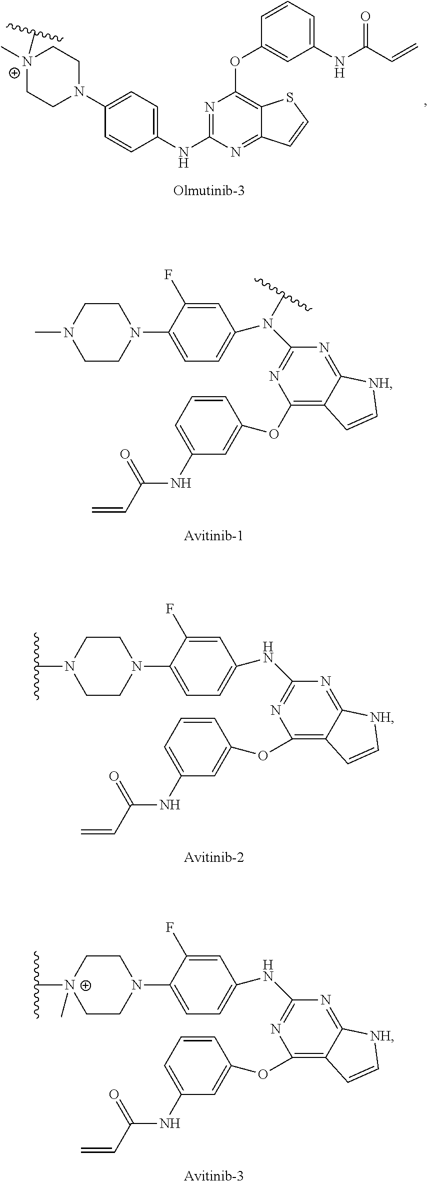

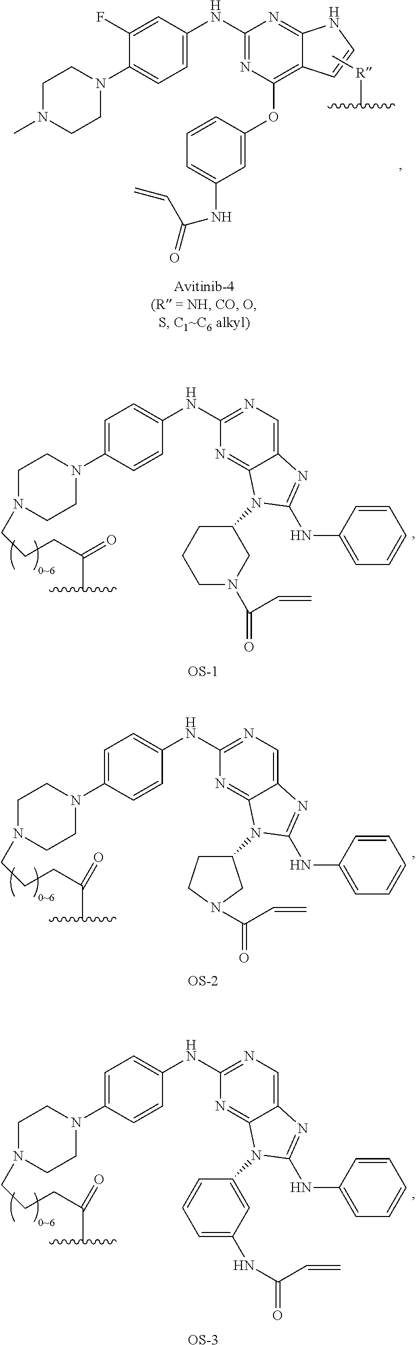

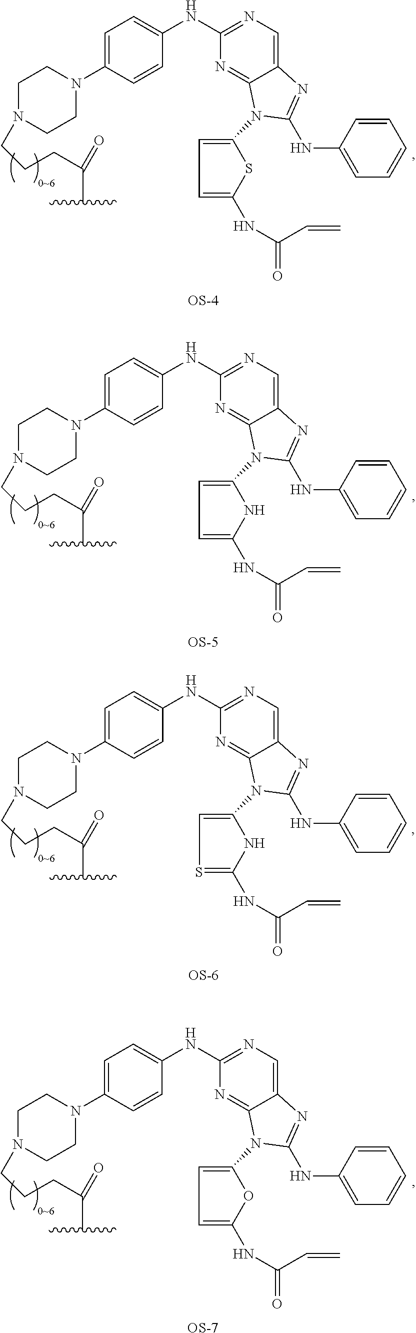

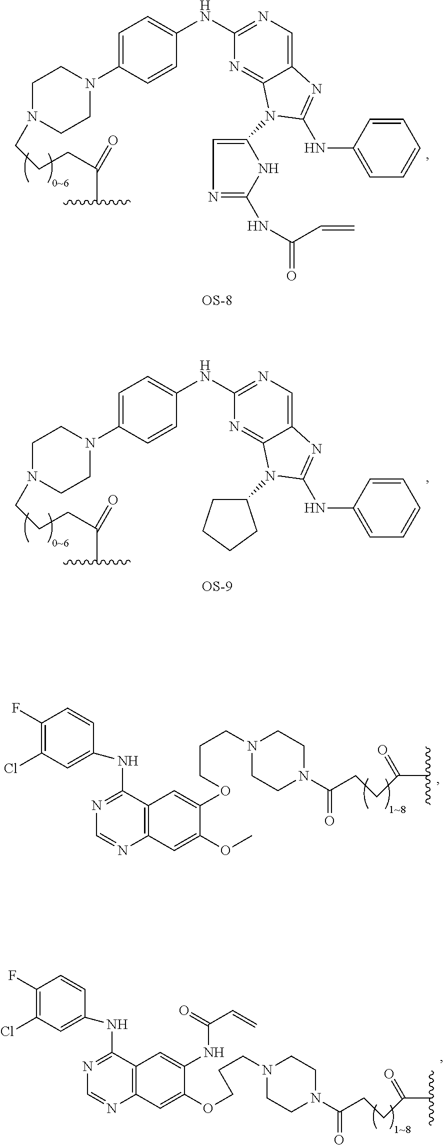

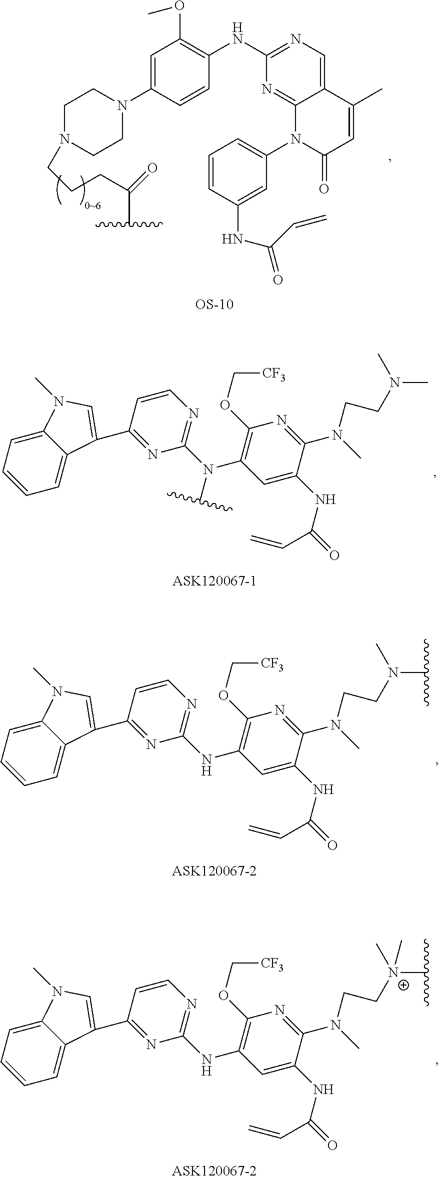

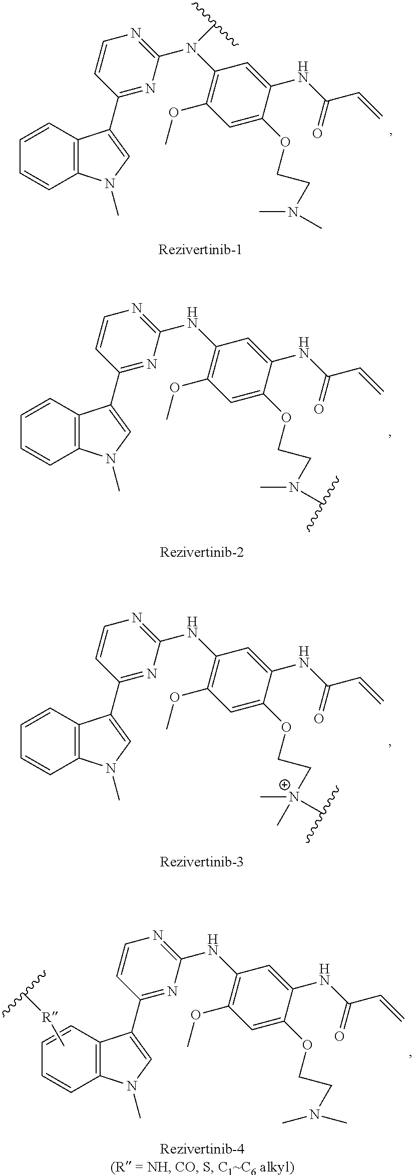

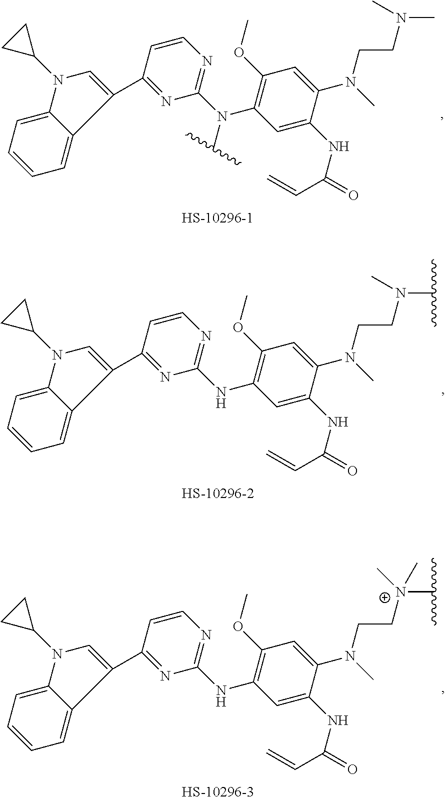 [SMILES: *N(c1nccc(-c2cn(C3CC3)c3ccccc23)n1)c1cc(NC(=O)C=C)c(N(C)CCN(C)C)cc1OC ; *N(C)CCN(C)c1cc(OC)c(Nc2nccc(-c3cn(C4CC4)c4ccccc34)n2)cc1NC(=O)C=C ; *[N+](C)(C)CCN(C)c1cc(OC)c(Nc2nccc(-c3cn(C4CC4)c4ccccc34)n2)cc1NC(=O)C=C]

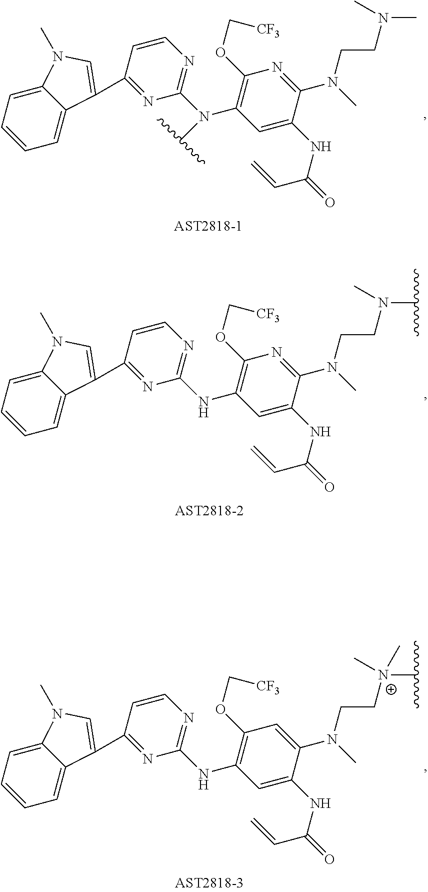 [SMILES: *N(c1nccc(-c2cn(C)c3ccccc23)n1)c1cc(NC(=O)C=C)c(N(C)CCN(C)C)nc1OCC(F)(F)F ; *N(C)CCN(C)c1nc(OCC(F)(F)F)c(Nc2nccc(-c3cn(C)c4ccccc34)n2)cc1NC(=O)C=C ; *[N+](C)(C)CCN(C)c1cc(OCC(F)(F)F)c(Nc2nccc(-c3cn(C)c4ccccc34)n2)cc1NC(=O)C=C]

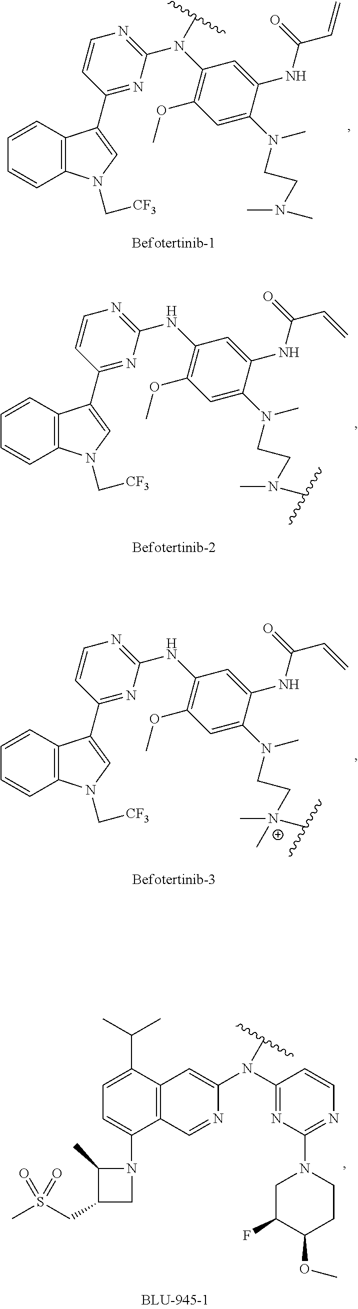

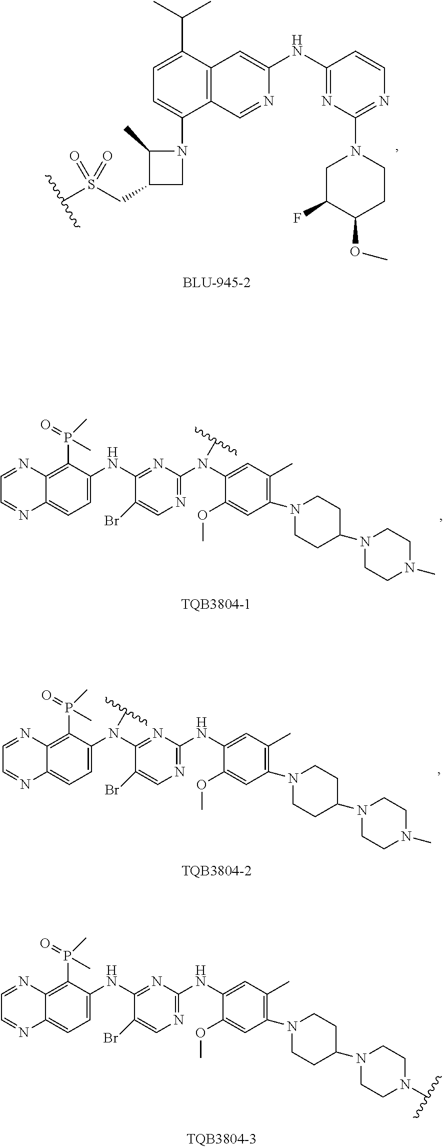 [SMILES: *S(=O)(=O)C[C@H]1CN(c2ccc(C(C)C)c3cc(Nc4ccnc(N5CC[C@@H](OC)[C@@H](F)C5)n4)ncc23)[C@@H]1C ; *N(c1ncc(Br)c(Nc2ccc3nccnc3c2P(C)(C)=O)n1)c1cc(C)c(N2CCC(N3CCN(C)CC3)CC2)cc1OC ; *N(c1ccc2nccnc2c1P(C)(C)=O)c1nc(Nc2cc(C)c(N3CCC(N4CCN(C)CC4)CC3)cc2OC)ncc1Br ; *N1CCN(C2CCN(c3cc(OC)c(Nc4ncc(Br)c(Nc5ccc6nccnc6c5P(C)(C)=O)n4)cc3C)CC2)CC1]

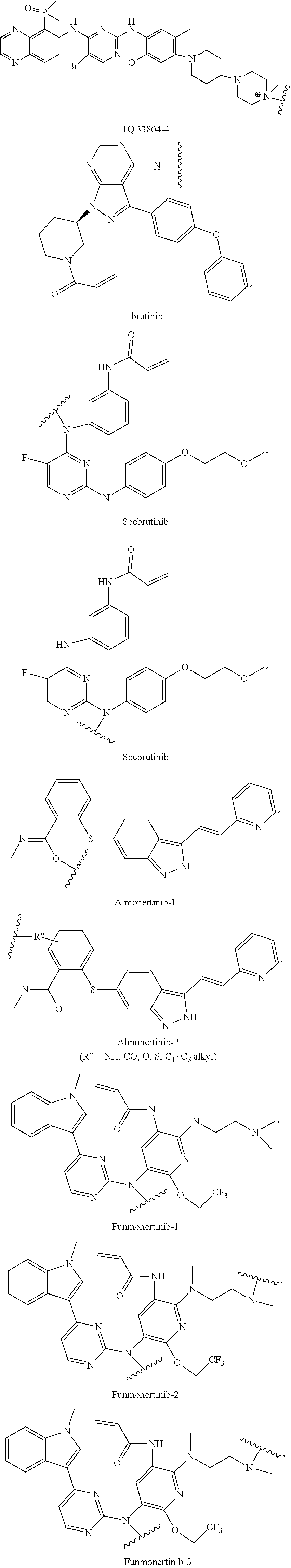

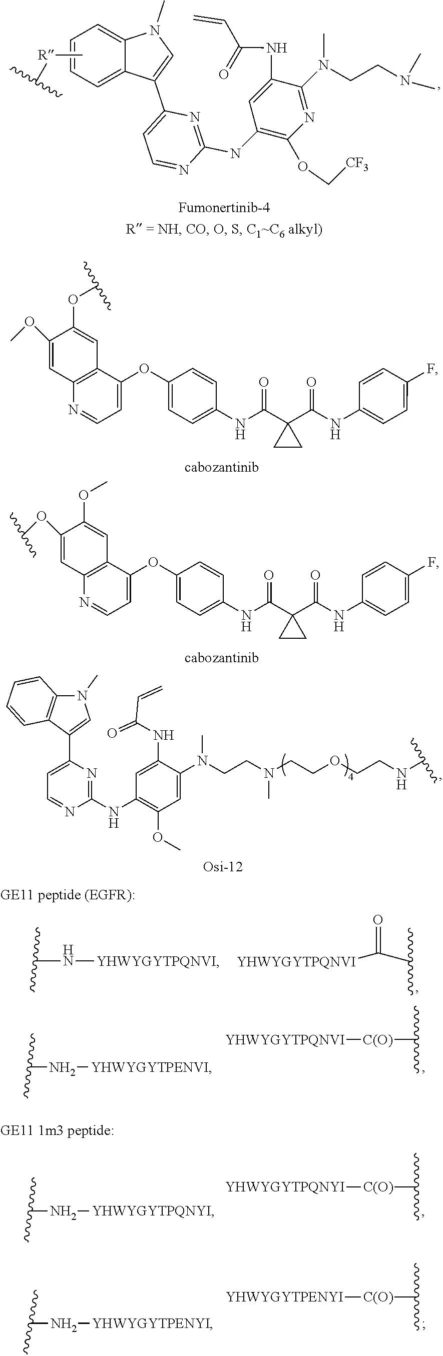

or hydrated salts; or the polymorphic crystalline structures of these compounds; or the optical isomers, racemates, diastereomers or enantiomers; wherein [symbol] is the linkage site.
5). A PLK1 inhibitor: Polo-like kinase 1 (PLK1), also known as Serine/threonine-protein kinase PLK1, or serine/threonine-protein kinase 13 (STPK13), is an enzyme that in humans is encoded by the PLK1 (polo-like kinase 1) gene. PLK1 consists of 603 amino acids and is 66 kDa and it is an early trigger for G2/M transition. PLK1 is considered a proto-oncogene, whose overexpression is often observed in tumor cells. PLK1 is being studied as a target for cancer drugs. Many colon and lung cancers are caused by K-RAS mutations. These cancers are dependent on PLK1. When PLK1 expression was silenced with RNA interference in cell culture, K-RAS cells were selectively killed, without harming normal cells (Downward J, N Engl J Med, 361:922, 2009; Luo J, et al. Cell. 137 (5): 835-48, 2009). Some of the conjugatable structures of the PLK1 inhibitors are illustrated below:

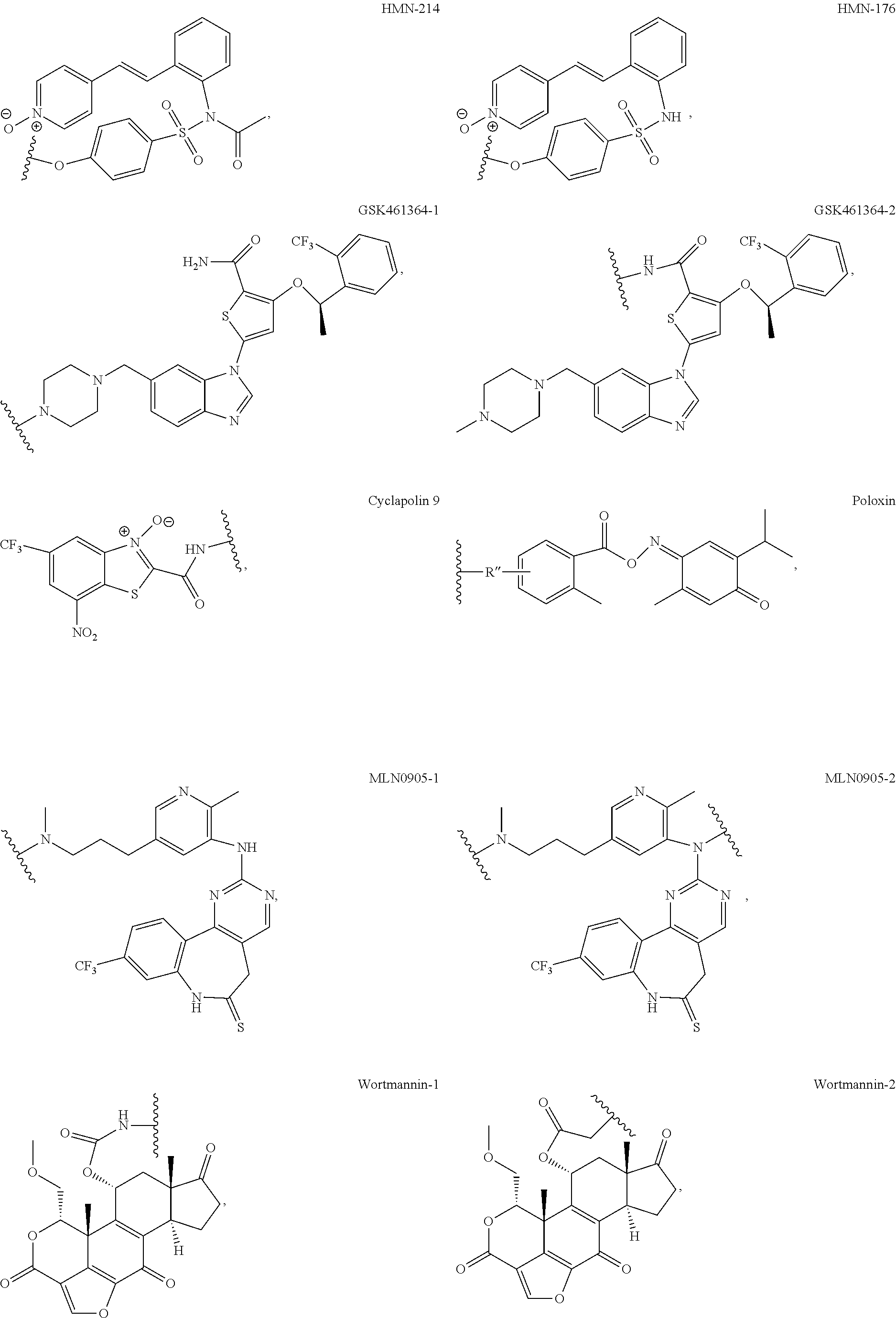

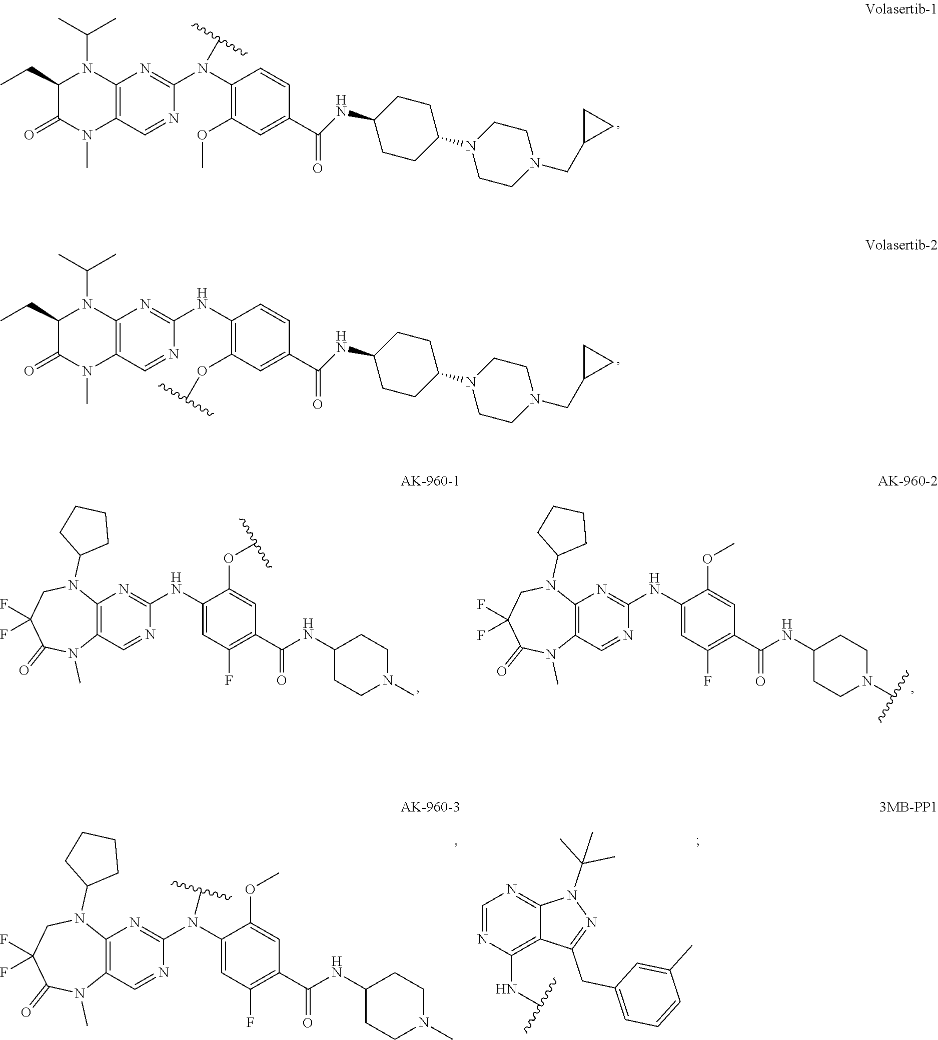 [SMILES: *N(c1ncc2c(n1)N(C(C)C)[C@H](CC)C(=O)N2C)c1ccc(C(=O)N[C@H]2CC[C@H](N3CCN(CC4CC4)CC3)CC2)cc1OC ; *Oc1cc(C(=O)N[C@H]2CC[C@H](N3CCN(CC4CC4)CC3)CC2)ccc1Nc1ncc2c(n1)N(C(C)C)[C@H](CC)C(=O)N2C ; *Oc1cc(C(=O)NC2CCN(C)CC2)c(F)cc1Nc1ncc2c(n1)N(C1CCCC1)CC(F)(F)C(=O)N2C ; *N1CCC(NC(=O)c2cc(OC)c(Nc3ncc4c(n3)N(C3CCCC3)CC(F)(F)C(=O)N4C)cc2F)CC1 ; *N(c1ncc2c(n1)N(C1CCCC1)CC(F)(F)C(=O)N2C)c1cc(F)c(C(=O)NC2CCN(C)CC2)cc1OC ; *Nc1ncnc2c1c(Cc1cccc(C)c1)nn2C(C)(C)C]

or hydrated salts; or the polymorphic crystalline structures of these compounds; or the optical isomers, racemates, diastereomers or enantiomers; wherein [symbol] is the linkage site.
6). A PI3K/AKT/mTOR inhibitor: It is a inhibitor of phosphoinositide 3-kinases/protein kinase B/mammalian target of rapamycin (PI3K/AKT/mTOR). Phosphoinositide 3-kinases (PI3Ks) are a family of enzymes that 3′-phosphorylate the inositol head group of membrane phosphoinositides and major cellular signaling molecules that regulate multiple cellular functions (Thorpe L M, et al, 2015 Nat Rev Cancer 15:7-24). The PI3K/AKT/mTOR signaling pathway has been described as one of the most commonly disrupted pathways in cancer, making it an attractive candidate for therapeutic intervention. The PI3K/AKT/mTOR pathway is crucial for cell motility, growth, survival, and metabolism in cancer (Hoxhaj G, Manning B D. Nat Rev Cancer (2020) 20(2): 74-88). The accessible targeted inhibitors for cancer patients with abnormal activation of the PI3K/AKT/mTOR pathway include everolimus (mTOR inhibitor), sirolimus (mTOR inhibitor), temsirolimus (mTOR inhibitor), vistusertib (mTOR inhibitor), sapanisertib (mTOR inhibitor), AZD8055 (mTOR inhibitor), PP242 (mTOR inhibitor), alpelisib (PI3K inhibitor), duvelisib (PI3K inhibitor), copanlisib (PI3K inhibitor), idelalisib (PI3K inhibitor), umbralisib (PI3K inhibitor), Sonolisib (PI3K inhibitor), Buparlisib (PI3K inhibitor), capivasertib (AKT inhibitor), ipatasertib (AKT inhibitor), MK-2206 (AKT inhibitor), GSK690693 (AKT inhibitor), uprosertib (AKT inhibitor), CH5132799 (PI3K inhibitor), pilaralisib (PI3K inhibitor), ZSTK474 (PI3K inhibitor), sonolisib (PI3K inhibitor), pictilisib (PI3K inhibitor), B591 (PI3K inhibitor), TG-100-115 (PI3K inhibitor), and RIDR-PI-103 (PI3K inhibitor), PX-866 (Wortmannin derivative, PI3K inhibitor), serabelisib (a PI3Kα inhibitor), GSK2636771 (a PI3 KB inhibitor), Zandelisib (a PI3Kδ inhibitor), AMG319 (a PI3Kδ inhibitor), linperlisib (a PI3Kδ inhibitor), parsaclisib (a PI3Kδ/γ inhibitor), leniolisib (a PI3Kδ inhibitor), eganelisib (a PI3Kγ inhibitor), tenalisib (a PI3Kδ/γ inhibitor), taselisib (a PI3Kα/δ/γ inhibitor), AZD8186 (a PI3 KB/δ inhibitor), and AZD8835 (a PI3Kδ/α inhibitor), dactolisib (PI3K/mTOR inhibitor), apitolisib (GDC-0980, PI3K/mTOR inhibitor), gedatolisib (PF-05212384, PI3K/mTOR inhibitor), bimiralisib (PQR309) (PI3K/mTOR inhibitor), paxalisib (GDC-0084) (PI3K/mTOR inhibitor), and voxtalisib (SAR245409, XL765) (PI3K/mTOR inhibitor). Examples of the conjugatable structures of the above inhibitors of PI3K/AKT/mTOR are illustrated below:

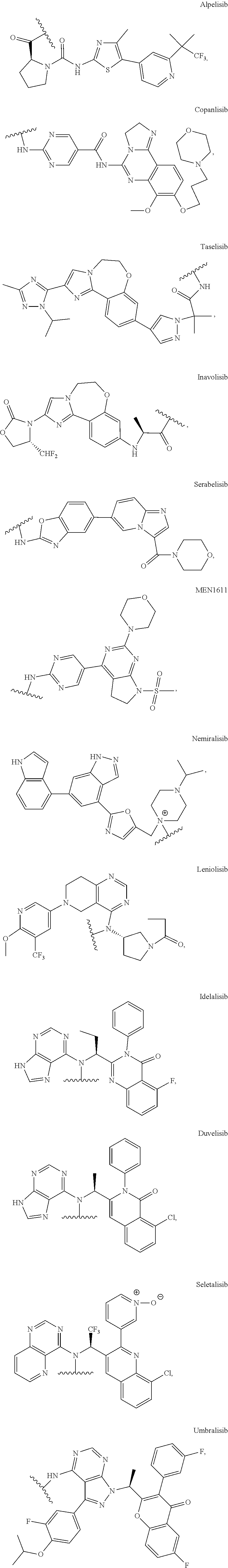

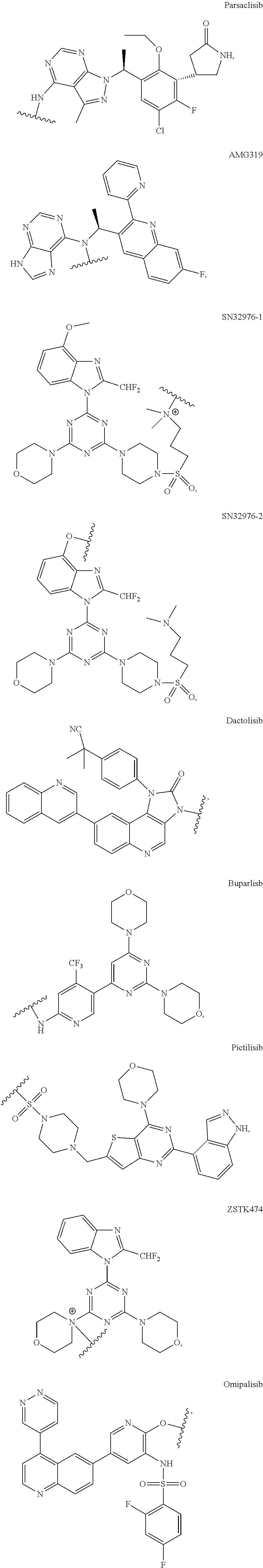

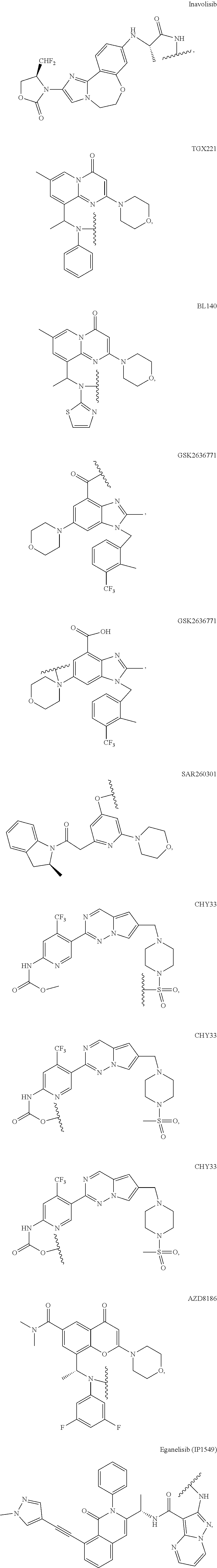

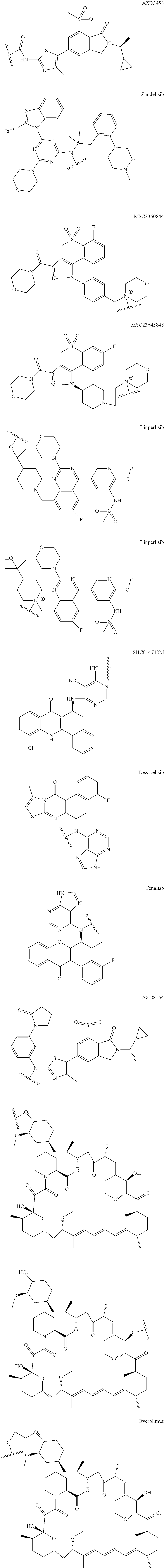

or hydrated salts; or the polymorphic crystalline structures of these compounds; or the optical isomers, racemates, diastereomers or enantiomers; wherein [symbol] is the linkage site.
7). A Histone deacetylase inhibitor (HDACI): Histone deacetylase mediates histone deacetylation and acts in concert with histone acetyltransferases (HATs) to regulate dynamic and reversible histone acetylation which modifies chromatin structure and function, affects gene transcription, thus, controlling multiple cellular processes. Inhibitors of HDACs can regulate transcription and inhibit proliferation of cancer cells by inducing cell cycle arrest, differentiation and/or apoptosis, among other major biological phenomena. The different mechanism(s) of action of HDAC inhibitors compared to conventional anti-neoplastic agents provides a possibility that HDAC inhibitors may be effective for refractory cancers. Accordingly, some structures of histone deacetylase inhibitors used in the patent are illustrated below:

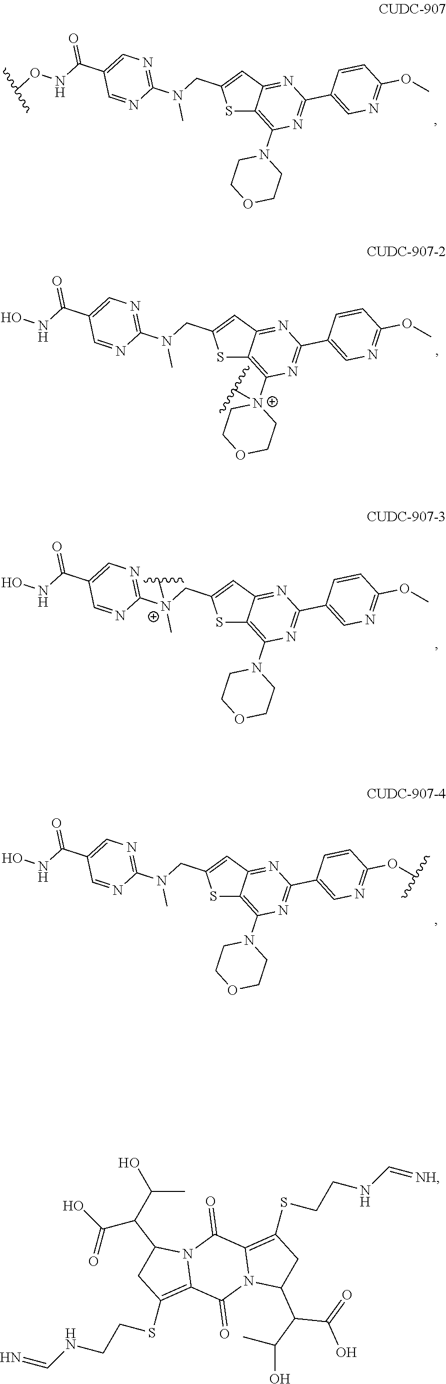 [SMILES: *ONC(=O)c1cnc(N(C)Cc2cc3nc(-c4ccc(OC)nc4)nc(N4CCOCC4)c3s2)nc1 ; *[N+]1(c2nc(-c3ccc(OC)nc3)nc3cc(CN(C)c4ncc(C(=O)NO)cn4)sc23)CCOCC1 ; *[N+](C)(Cc1cc2nc(-c3ccc(OC)nc3)nc(N3CCOCC3)c2s1)c1ncc(C(=O)NO)cn1 ; *Oc1ccc(-c2nc(N3CCOCC3)c3sc(CN(C)c4ncc(C(=O)NO)cn4)cc3n2)cn1 ; CC(O)C(C(=O)O)C1CC(SCCNC=N)=c2c(=O)n3c(c(=O)n21)=C(SCCNC=N)CC3C(C(=O)O)C(C)O]

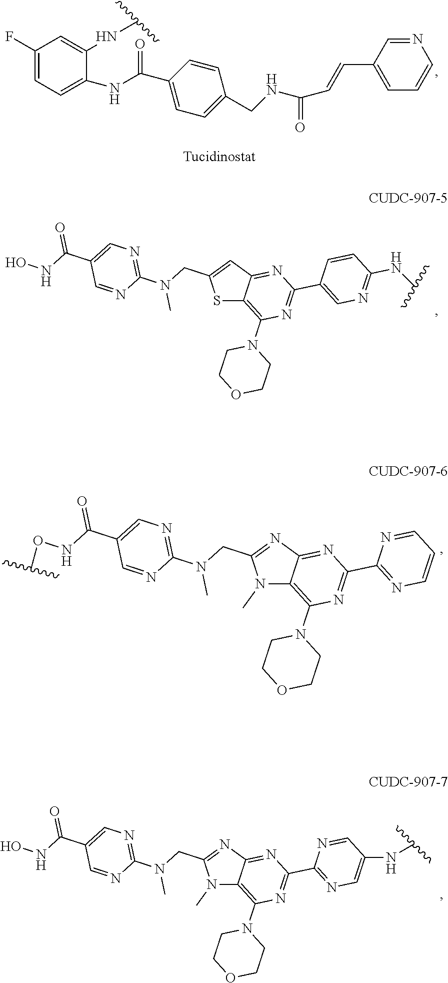 [SMILES: *Nc1cc(F)ccc1NC(=O)c1ccc(CNC(=O)/C=C/c2cccnc2)cc1 ; *Nc1ccc(-c2nc(N3CCOCC3)c3sc(CN(C)c4ncc(C(=O)NO)cn4)cc3n2)cn1 ; *ONC(=O)c1cnc(N(C)Cc2nc3nc(-c4ncccn4)nc(N4CCOCC4)c3n2C)nc1 ; *Nc1cnc(-c2nc(N3CCOCC3)c3c(n2)nc(CN(C)c2ncc(C(=O)NO)cn2)n3C)nc1]

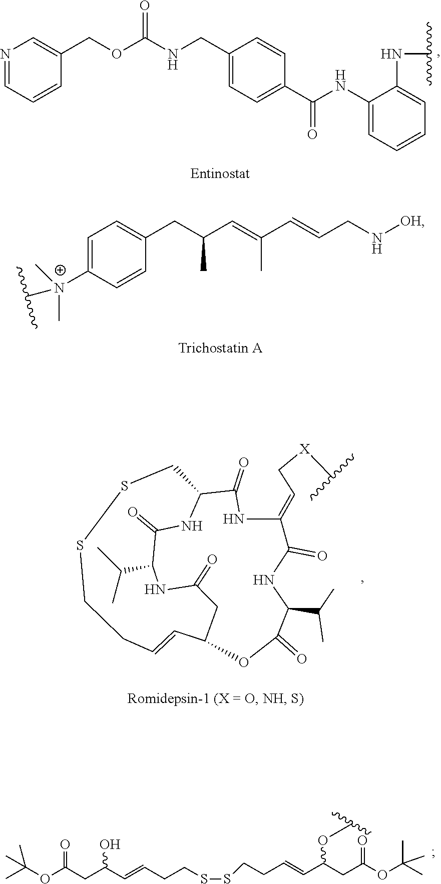

or hydrated salts; or the polymorphic crystalline structures of these compounds; or the optical isomers, racemates, diastereomers or enantiomers; wherein [symbol] is the linkage site.
8). An inhibitor of Nuclear factor kappa B (NF-κB): Nuclear factor kappa-light-chain-enhancer of activated B cells (NF-κB) is a protein complex that controls transcription of DNA, cytokine production and cell survival. NF-κB plays a key role in regulating the immune response to infection. Aberrant activation of NF-κB has been linked to cancer, inflammatory and autoimmune diseases, septic shock, viral infection, and improper immune development. Hence methods of inhibiting NF-κB signaling has potential therapeutic application in cancer and inflammatory diseases. Disulfiram, olmesartan and dithiocar-bamates can inhibit the nuclear factor-KB (NF-κB) signaling cascade (Cvek B, Dvorak Z (2007) Current Pharmaceutical Design. 13 (30): 3155-3167). NF-κB inhibitors such as (−)-DHMEQ, PBS-1086, IT-603 and IT-901 (Blakely C M, et al. (April 2015), Cell Reports. 11 (1): 98-110; Fabre C, et al. (2012) Clinical Cancer Research. 18 (17): 4669-4681; Shono Y, et al. (2016) Cancer Research. 76 (2): 377-389) has been discovered, but their applications are still limited. (−)-DHMEQ and PBS-1086 are irreversible binder to NF-κB while IT-603 and IT-901 are reversible binder. DHMEQ covalently binds to Cys 38 of p65 (Yamamoto M, et al (2008) J. Med Chemistry. 51 (18): 5780-5788). The conjugatable structures of inhibitors of NF-κB used for the present patent are illustrated below:

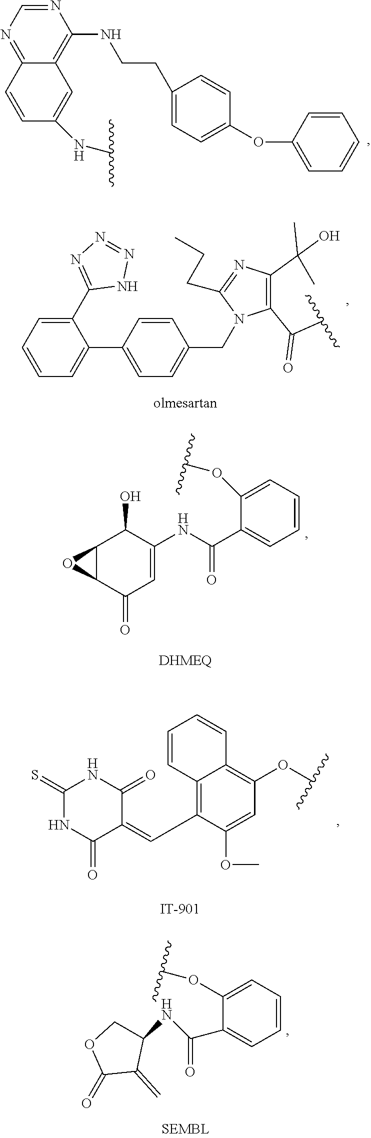 [SMILES: *Nc1ccc2ncnc(NCCc3ccc(Oc4ccccc4)cc3)c2c1 ; *C(=O)c1c(C(C)(C)O)nc(CCC)n1Cc1ccc(-c2ccccc2-c2nnn[nH]2)cc1 ; *Oc1ccccc1C(=O)NC1=CC(=O)[C@H]2O[C@H]2[C@H]1O ; *Oc1cc(OC)c(C=C2C(=O)NC(=S)NC2=O)c2ccccc12 ; *Oc1ccccc1C(=O)N[C@@H]1COC(=O)C1=C]

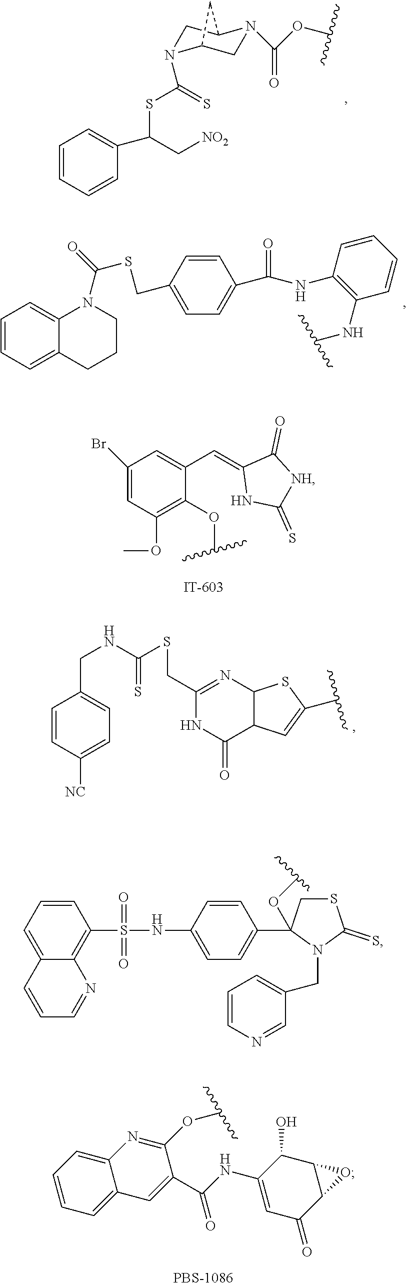

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the linkage site.
9). PD-L1/PD-1 ligand: Targeting programmed cell death 1 (PD-1) expressed on the surface of T cells and the programmed cell death 1 ligand 1 (PD-L1) expressed on cancer cells using immune checkpoint molecules, is an important strategy in this field [Y. Han, et al, Am J Cancer Res. 2020, 10, 727], since high PD-L₁ expression in tumor microenvironment is frequently observed in many types of cancers including Hodgkin's lymphoma, breast cancer, renal cell carcinoma, melanoma, lung cancer, gastric cancer, and hepatoma. In the tumor microenvironment, PD-L1 binds to PD-1 leading to T cell dysfunction, whereas blockage of their interactions recovers the T cell's activity of destroying tumor cells. Specifically, monoclonal antibodies (mAbs) that target either PD-1 or PD-L1 can block the interaction between them, hence promoting T cell activation and enabling T cell-mediated tumor cell death [S. Bagchi, et al, Annu Rev Pathol. 2021, 16, 223]. However, PD-1/PD-L1-based immunotherapy proved unsatisfactory in solid tumors due to the compact and hypoxic tumor microenvironment [M. Binnewies, et al, Nat Med. 2018, 24, 541], thereby hindering its broad implementation in cancer treatment [C. Zhang, et al, Cancer Lett. 2023, 562, 216182; C. Zhang, et al, Cancer Lett. 2023, 562, 216182]. Clinical studies reported that the blockade of PD-1/PD-L1 interactions can boost T cell-mediated antitumor responses, generate durable clinical responses, and prolong patient survival rate. Natural product-derived PD-1/PD-L₁ inhibitors of small molecules include: Amphotericin B, Bacitracin, Everolimus, Clarithromycin, Cyclosporin A, Actinomycin D, Cynocobalamin, Bryostatin, Candicidin, Geldanamycin, Ivermectin Bla, Macbecin, Metocurine, Monocrotaline, Nystatin, Olerixafor, Sirolimus, Troleandomycin, Rifampin, Rifabutin, Rifapentine, Rifamycin SV, Formyl rifamycin, Rafaximin, Gramicidin S, Gramicidin S derivative, Kaempferol, Kaempferol-7-O-rhamnoside, Cosmosiin, Apigenin, Eriodictyol, Fisetin, Glyasperin C, Caffeoylquinic acid, 3-O-Caffeoylquinic acid, 4-O-Caffeoylquinic acid, 5-O-Caffeoylquinic acid, Ellagic acid, BMS202, ZINC 67,902,090 ((3S,3aR,6S,6aR)-N6-[4-(3-fluorophenyl)-pyrimidin-2-yl]-N3-(2-pyridylmethyl)-2,3,3a,5,6,6a-hexahydrofu), ZINC12,529,904 (1-isopropyl-3-[(3S,5S)-1-methyl-5-[3-(2-naphthyl)-1,2,4-oxadiazol-5-yl]pyrrolidin-3-yl]urea) and they may have the synergy or enhanced therapeutic effects when are jointly conjugated with a cytotoxic agents as the invention indicated. The conjugatable structures of PD-L1/PD-1 ligands are illustrated below:

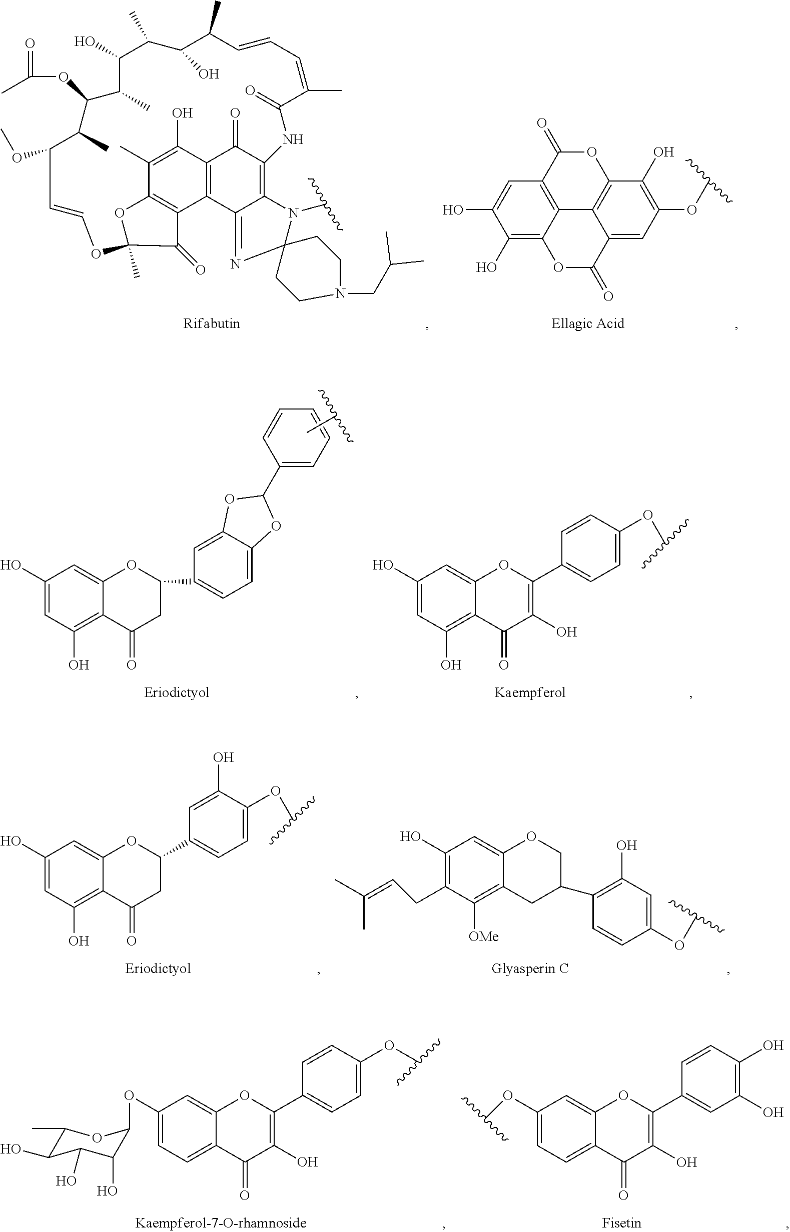

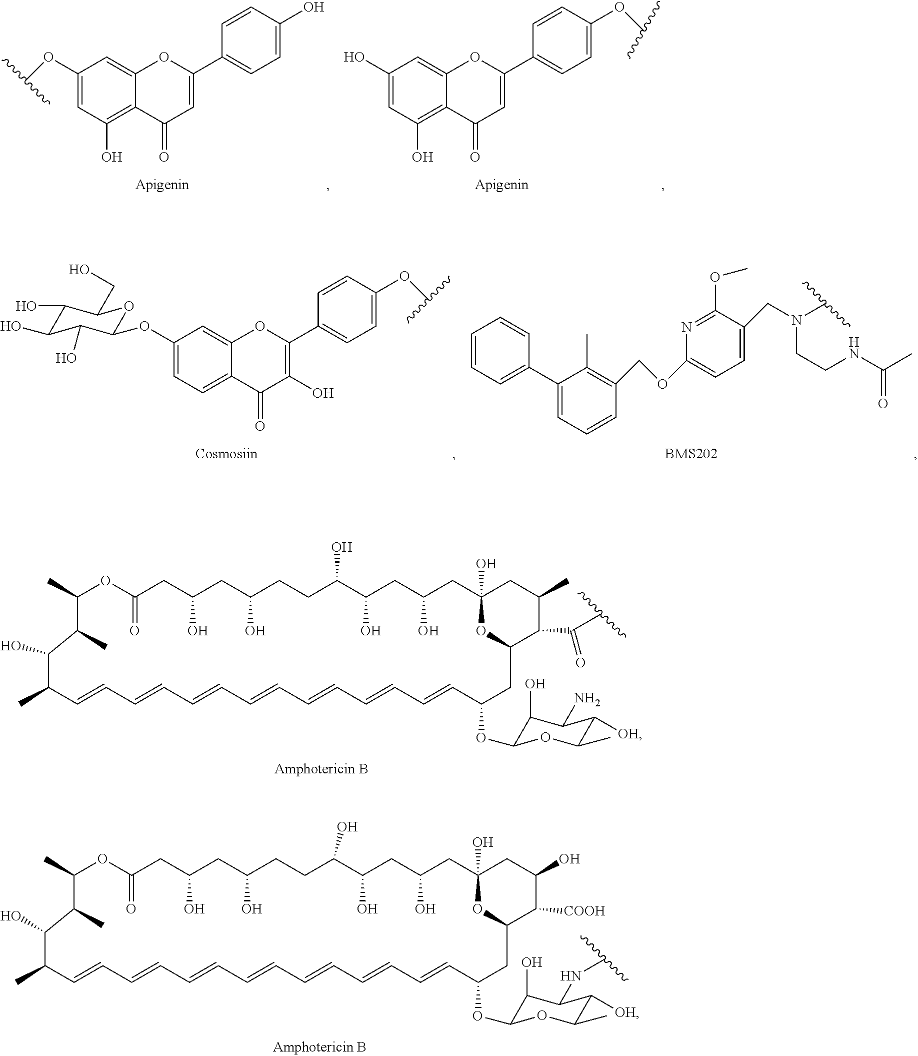

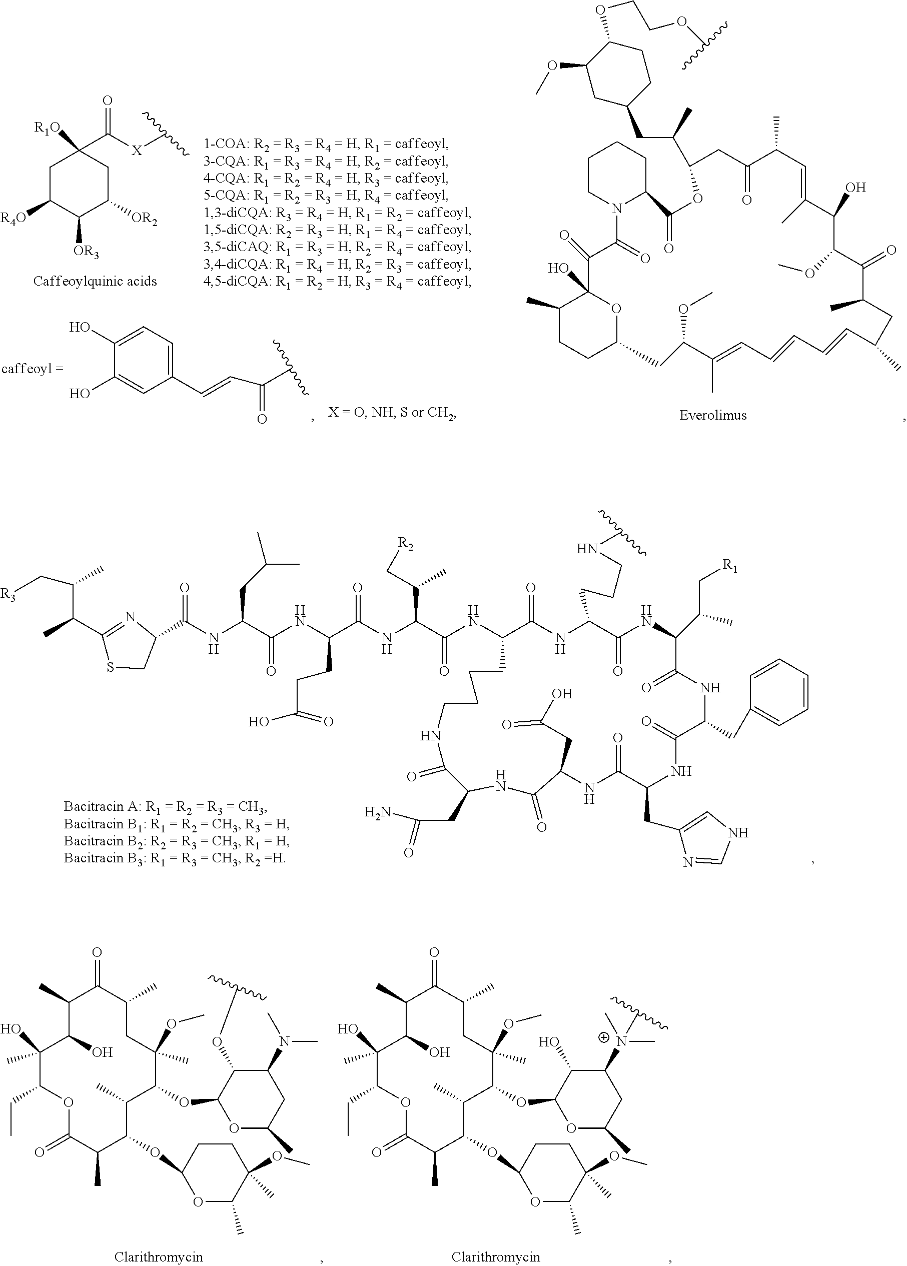

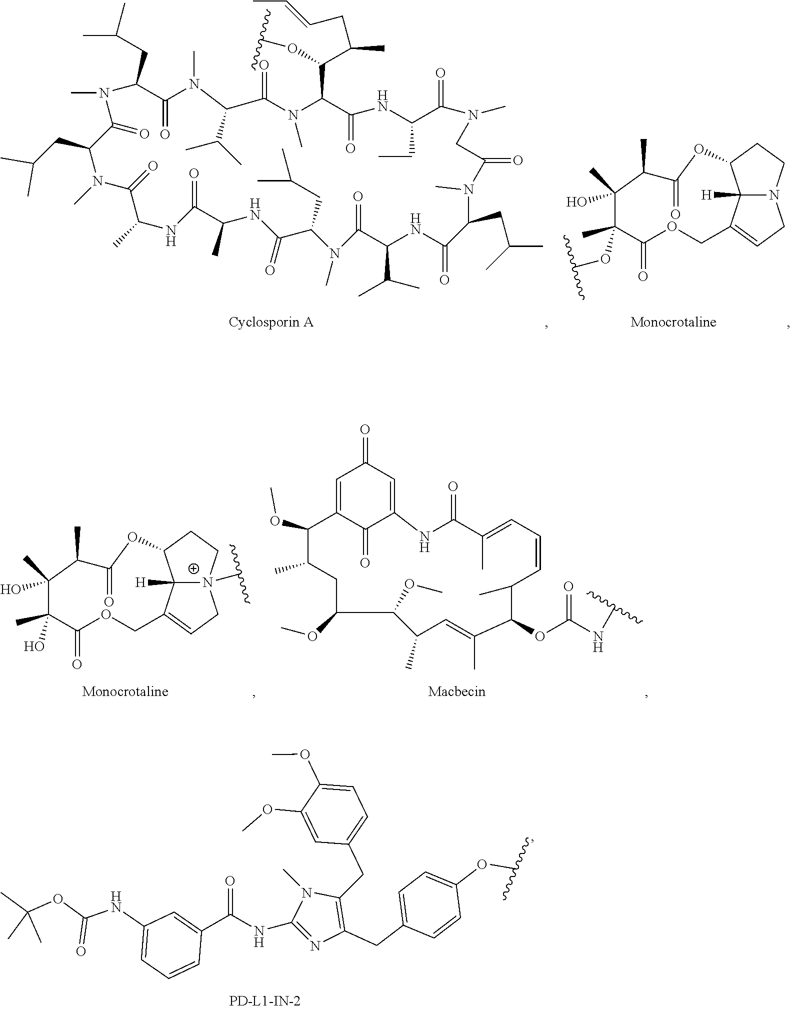

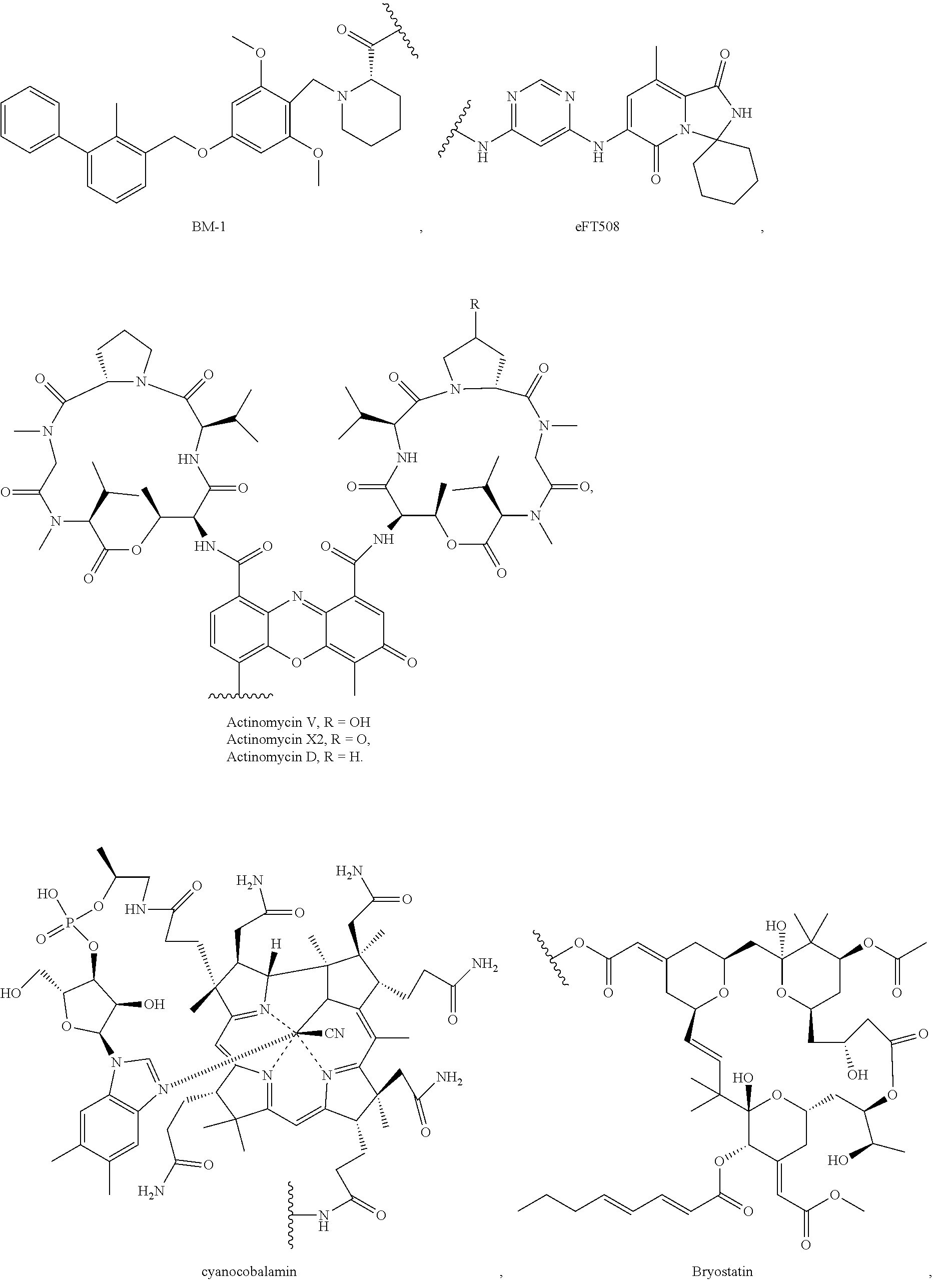

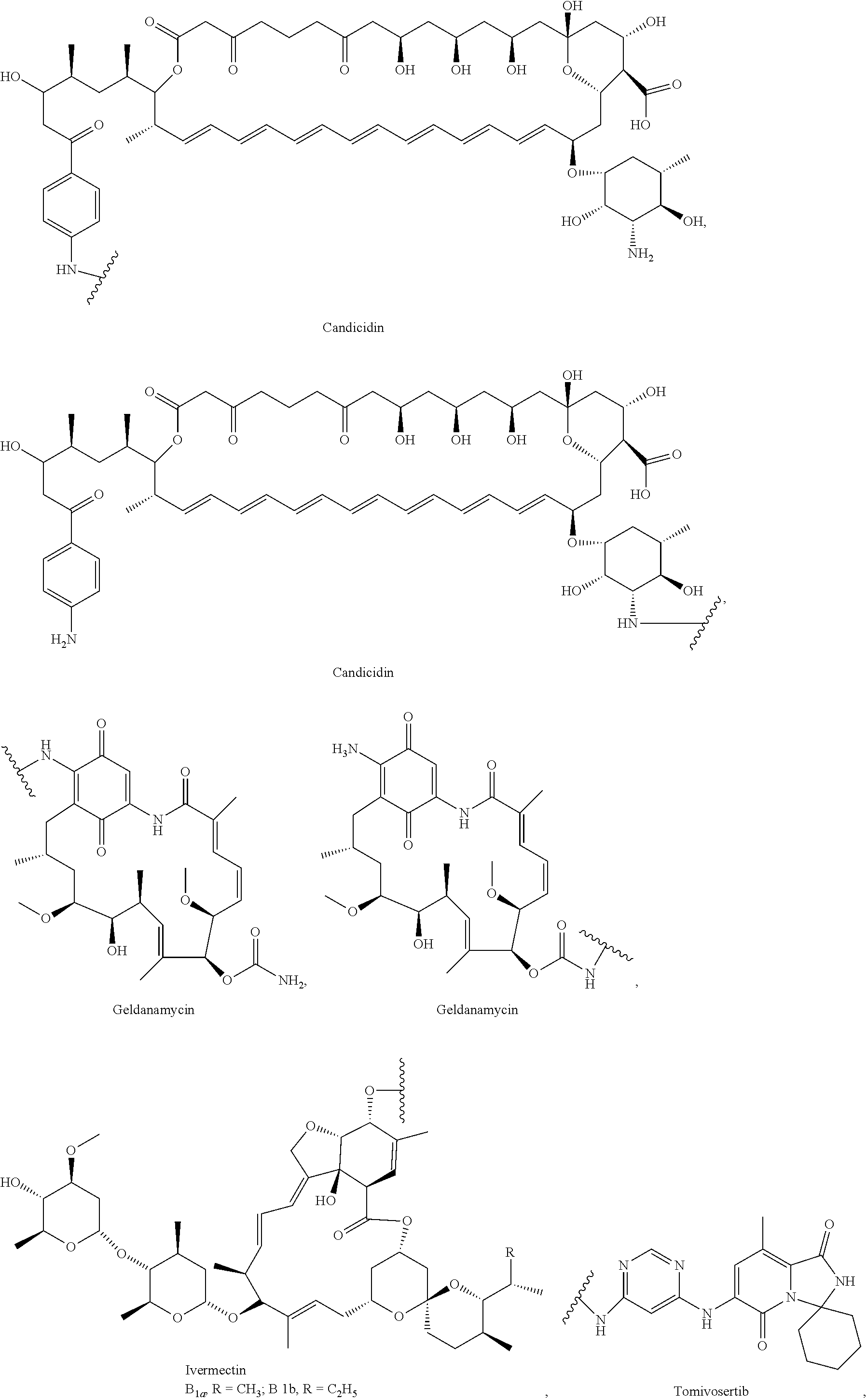

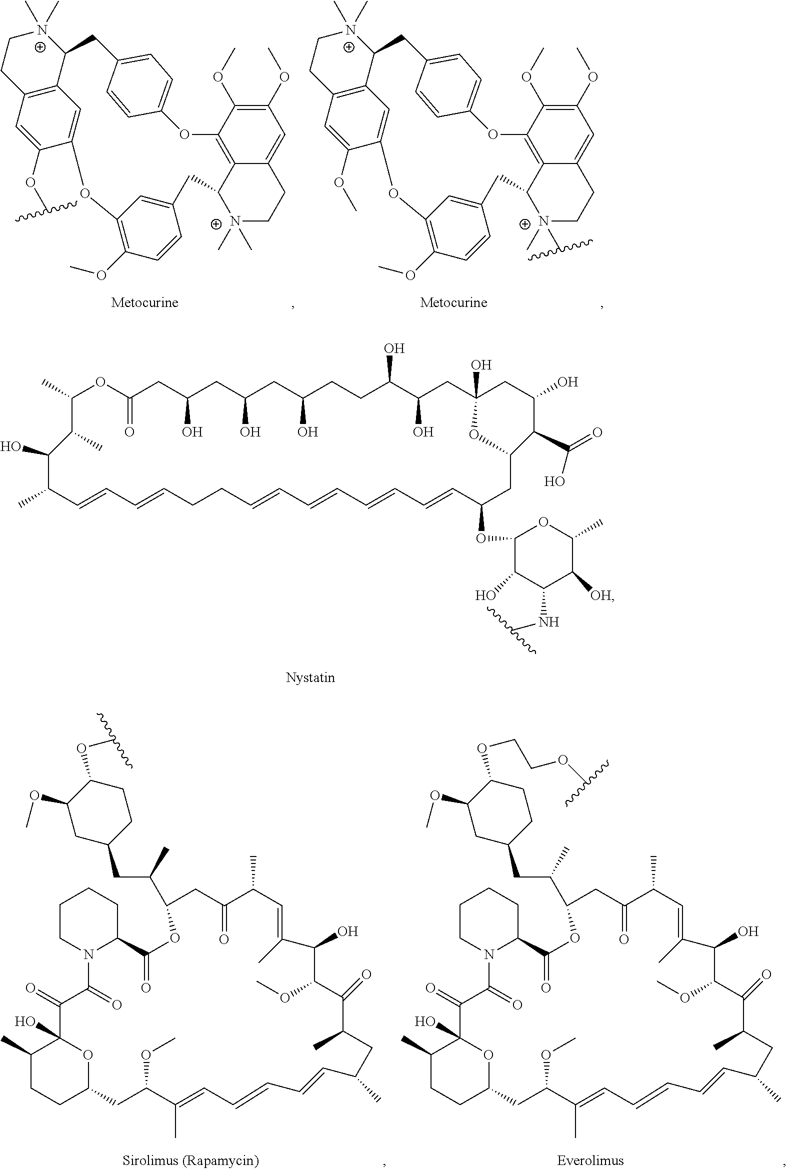

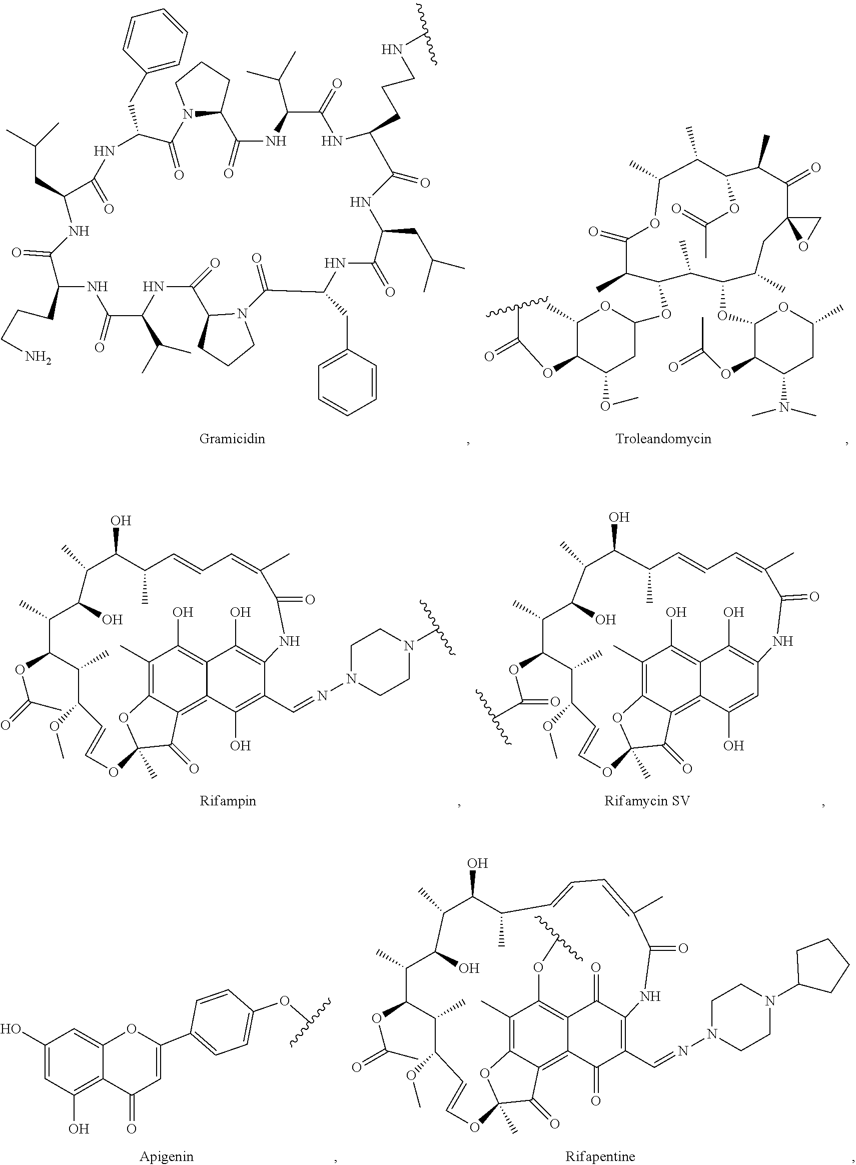

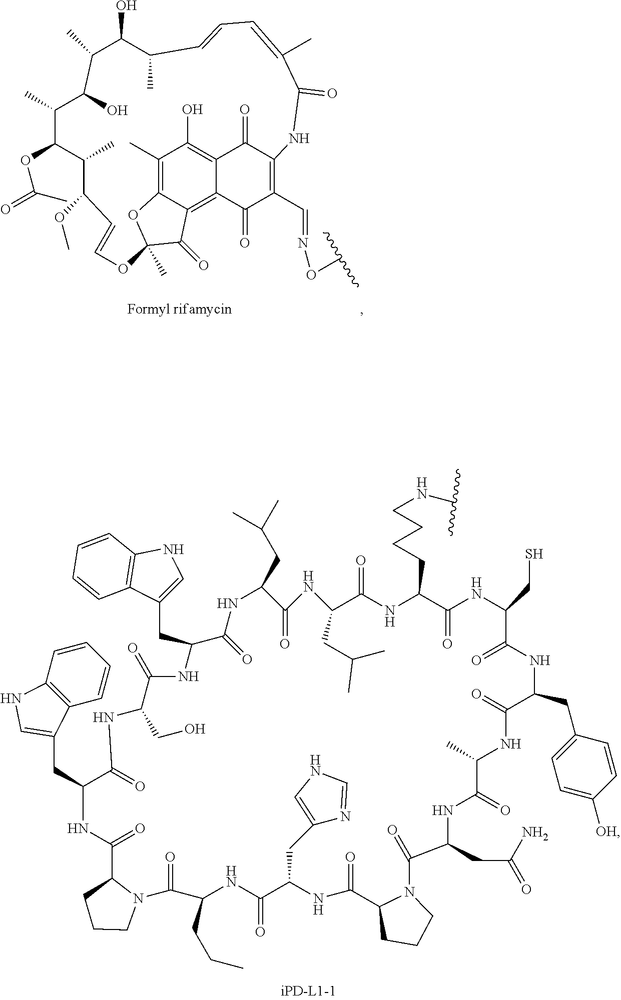 [SMILES: *O/N=C/C1=C2NC(=O)/C(C)=C\C=C\[C@H](C)[C@H](O)[C@@H](C)[C@@H](O)[C@@H](C)[C@H](OC(C)=O)[C@H](C)[C@@H](OC)/C=C/O[C@@]3(C)Oc4c(C)c(O)c(c(c4C3=O)C1=O)C2=O ; *NCCCC[C@@H]1NC(=O)[C@H](CC(C)C)NC(=O)[C@H](CC(C)C)NC(=O)[C@H](Cc2c[nH]c3ccccc23)NC(=O)[C@H](CO)NC(=O)[C@H](Cc2c[nH]c3ccccc23)NC(=O)[C@@H]2CCCN2C(=O)[C@H](CCC)NC(=O)[C@H](Cc2c[nH]cn2)NC(=O)[C@@H]2CCCN2C(=O)[C@H](CC(N)=O)NC(=O)[C@H](C)NC(=O)[C@H](Cc2ccc(O)cc2)NC(=O)[C@H](CS)NC1=O]

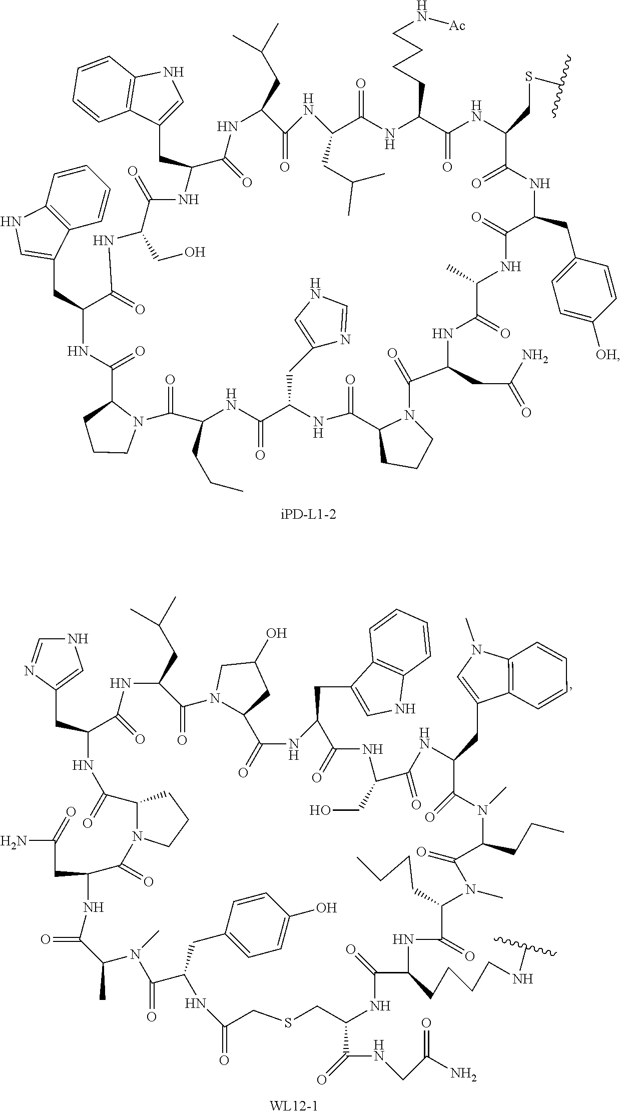

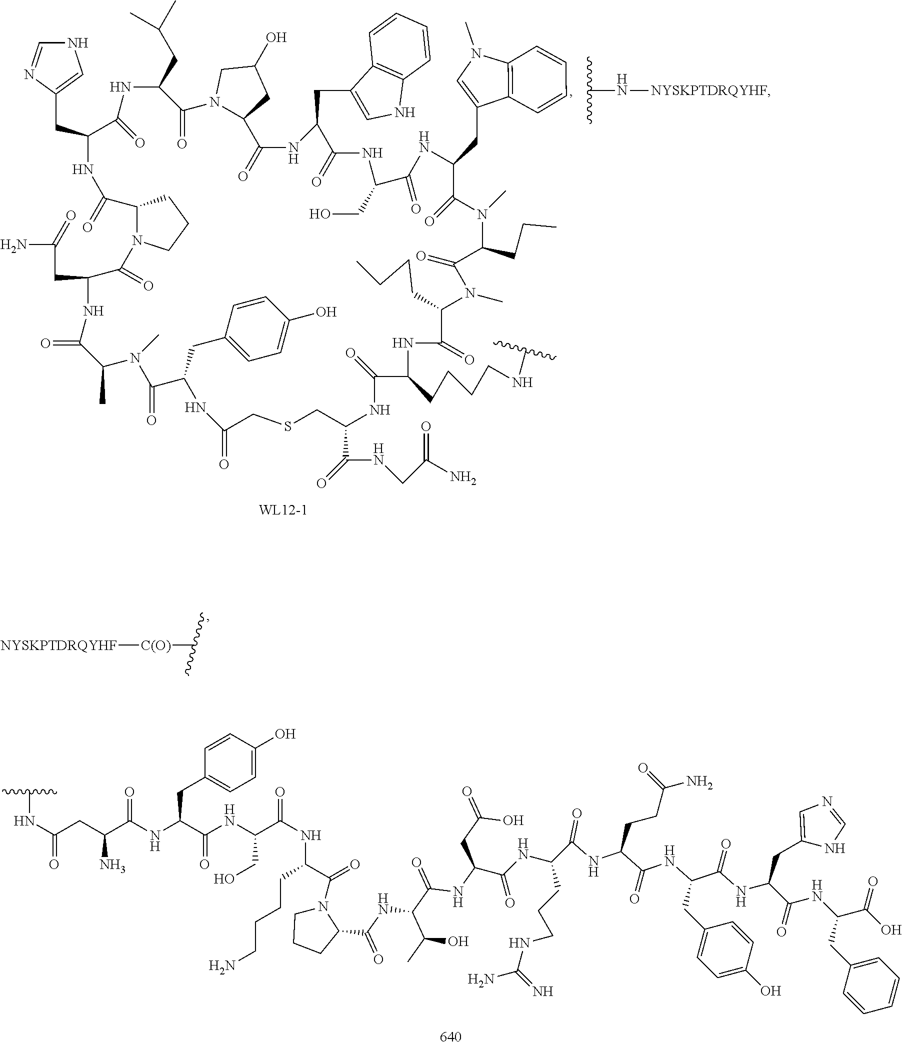

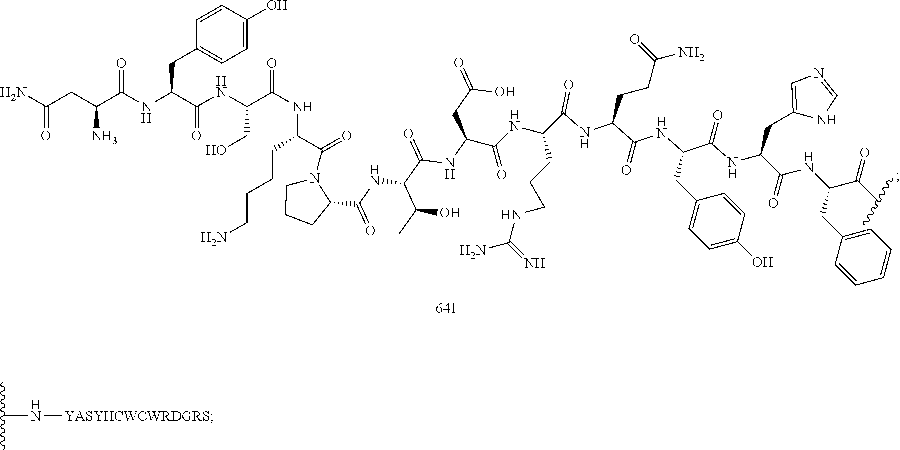

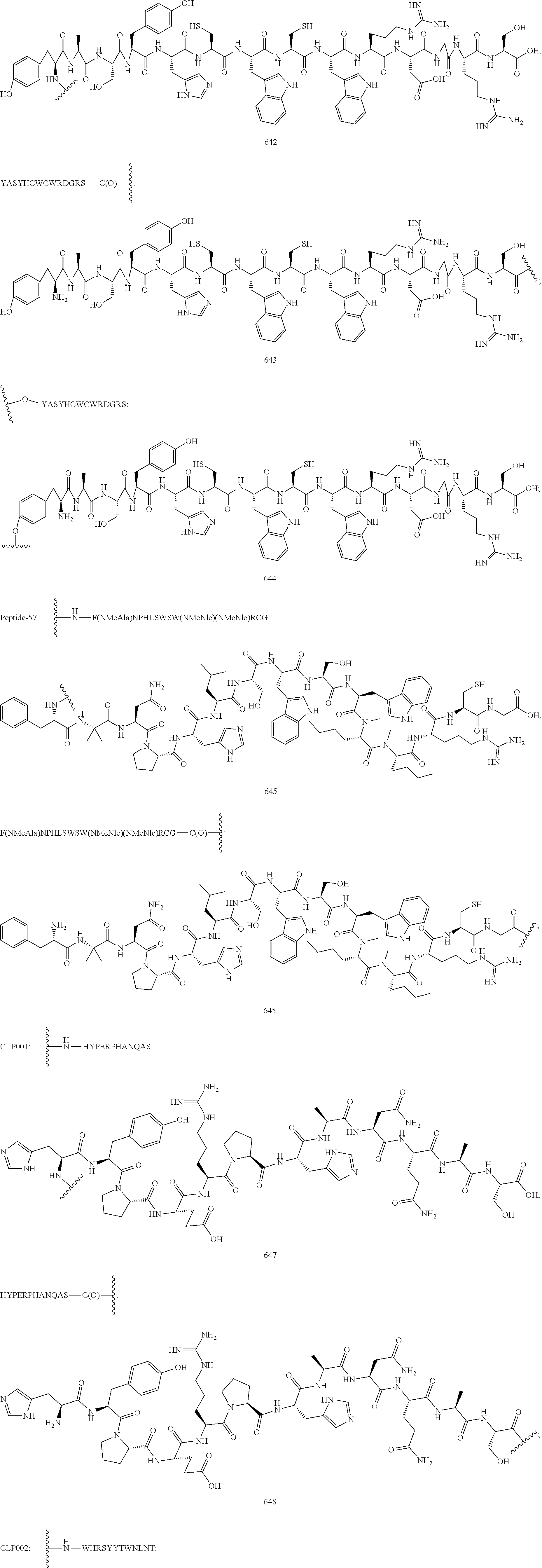

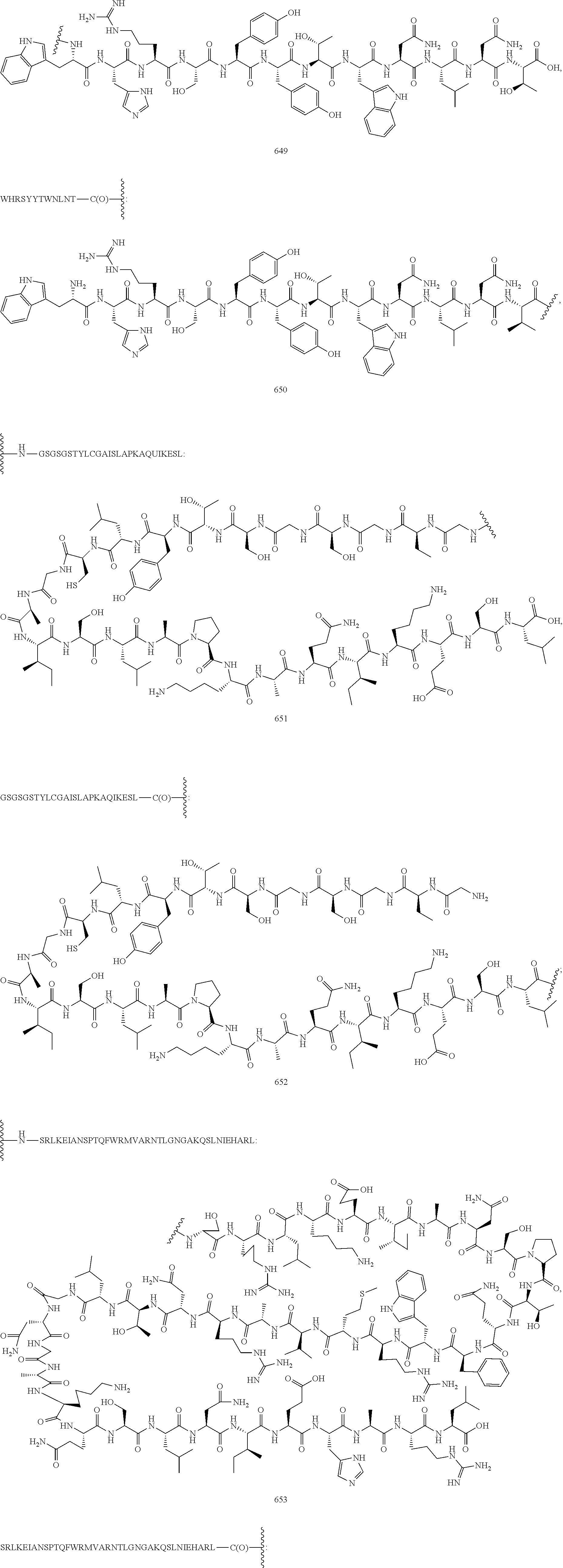

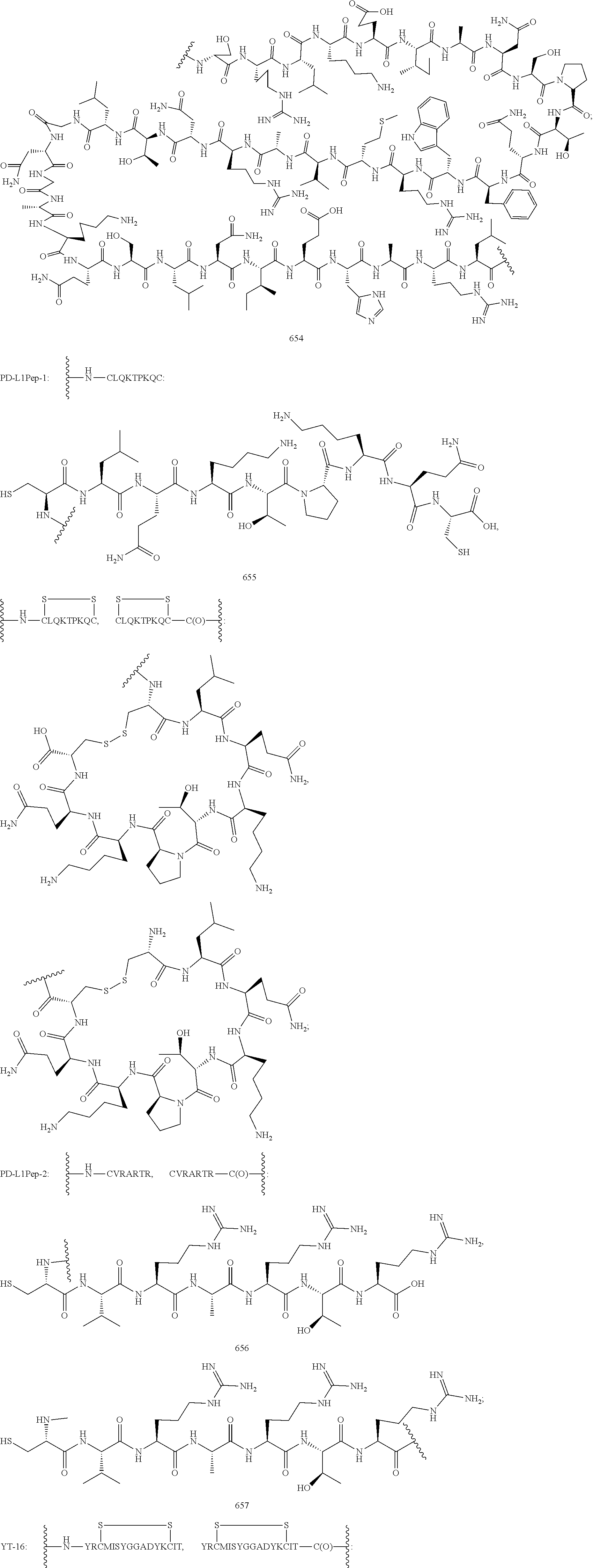

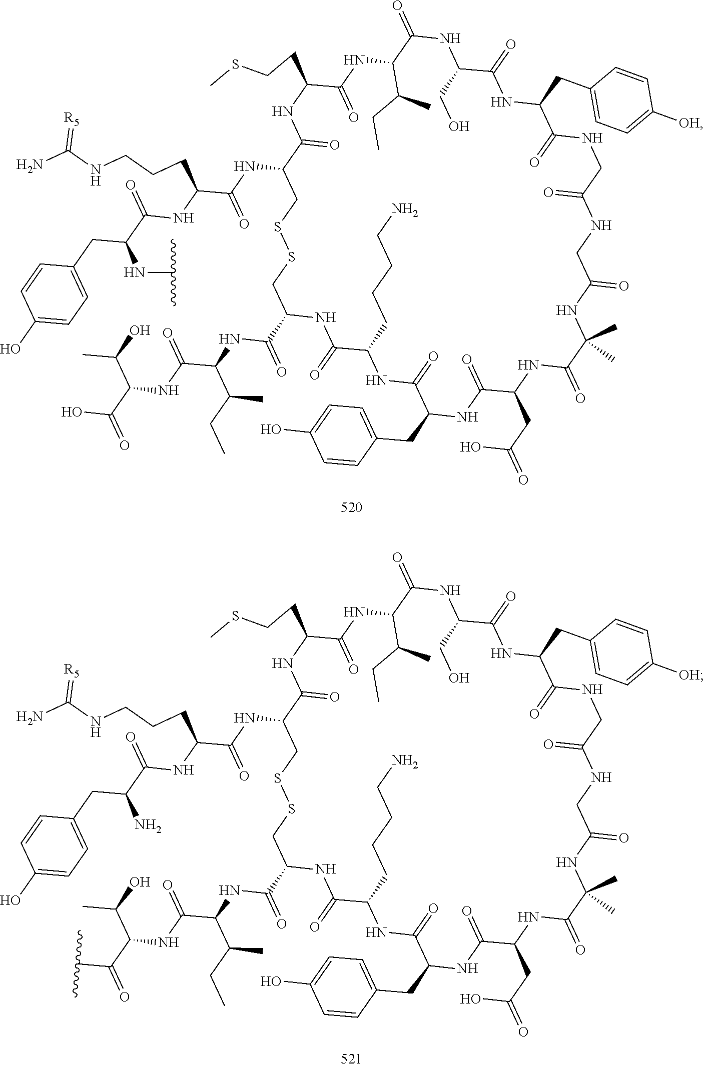

or hydrated salts; or the polymorphic crystalline structures of these compounds; or the optical isomers, racemates, diastereomers or enantiomers; wherein [symbol] is the linkage site.
10). A Kinesin spindle protein (KSP) inhibitor: Kinesin spindle proteins (KSP or Eg5) belong to the microtubule kinesin superfamily and they are responsible for the establishment of bipolar mitotic spindles that mediate cell division [C. Balakumar, et al, J. Biomol. Struct. Dyn., 36 (2018), pp. 3687-3704]. KSP plays a key role in spindle pole separation, formation of a bipolar mitotic spindle, as well as centrosome separation and maturation. Inhibition of KSP has the potential to provide anti-tumor activity while avoiding peripheral neuropathy associated with some microtubule-targeted drugs. Inhibition of KSP can cause cell death. Because of the high expression of KSPs in proliferation/cancer cells, it has become a drug target for tumor chemotherapy, although most KSP inhibitors have high toxicity and short half-life. There are several KSP inhibitors (e.g. Arry-520, LY2523355, 4SC-205, ALN-VSP02, Ispinesib, AZD4877, SB743921, ARQ621, MK-0731) that had entered clinical trials as anti-neoplastic agents against a variety of tumors and the results for the various trials were encouraging. However, many of the phase II or phase III clinical trials of kinesin spindle protein (KSP) inhibitors have failed due to issues such as high toxicity and a short circulation half-life in vivo. Thus the antibody-drug conjugate (ADC) technology was applied to a KSP inhibitor, SB-743921, which was coupled with the HER2-specific antibody trastuzumab using a cathepsin B-dependent valine-alanine (Val-Ala, VA) dipeptide-type linker to generate H2-921. Ex vivo and in vivo analyses of H2-921 showed an increased half-life of SB-743921 and prolonged contact time with tumor cells. But the tumor inhibition by H₂-921 was not better by the positive control drug T-DM1, the traditional ADC (Yiquan Li, et al, Biomaterials 2023, 301:122258). Therefore the conjugation of both KSP inhibitors and the cytotoxic payloads to an antibody has reasonable synergy in targeted treatment as the invention indicated. The conjugatable structures of kinesin spindle protein (KSP) inhibitors used for the patent are illustrated below:

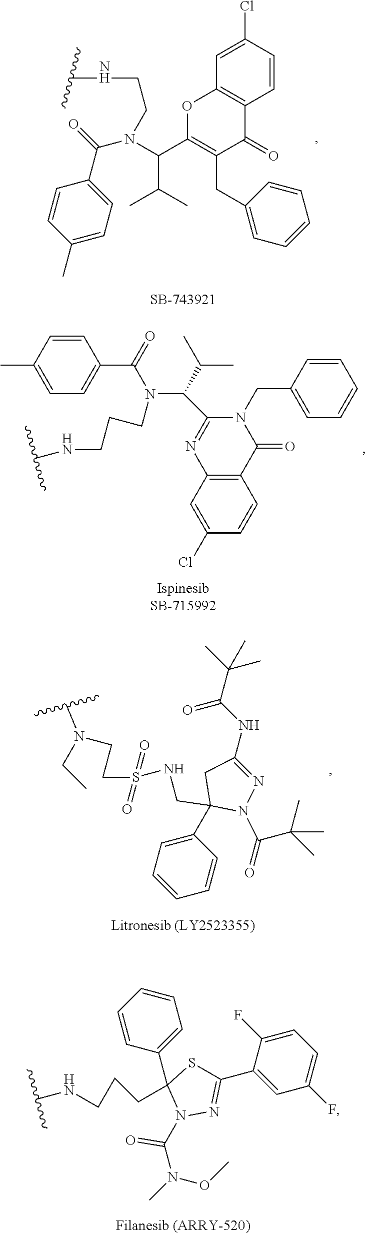 [SMILES: *NCCN(C(=O)c1ccc(C)cc1)C(c1oc2cc(Cl)ccc2c(=O)c1Cc1ccccc1)C(C)C ; *NCCCN(C(=O)c1ccc(C)cc1)[C@@H](c1nc2cc(Cl)ccc2c(=O)n1Cc1ccccc1)C(C)C ; *N(CC)CCS(=O)(=O)NCC1(c2ccccc2)CC(NC(=O)C(C)(C)C)=NN1C(=O)C(C)(C)C ; *NCCCC1(c2ccccc2)SC(c2cc(F)ccc2F)=NN1C(=O)N(C)OC]

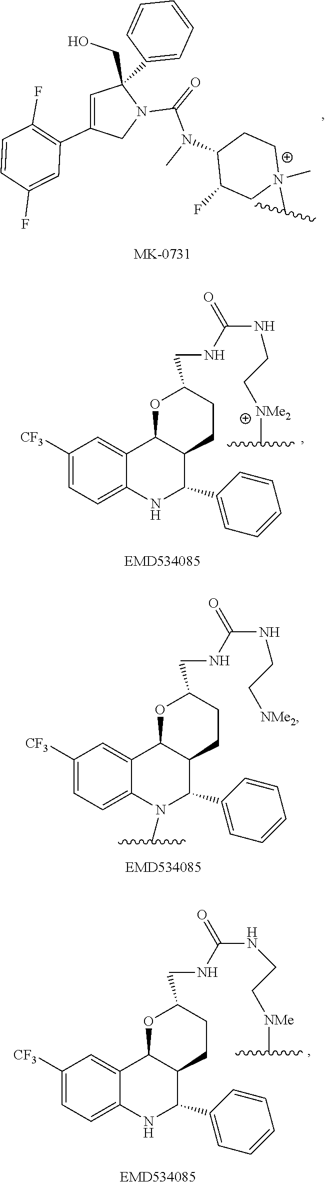

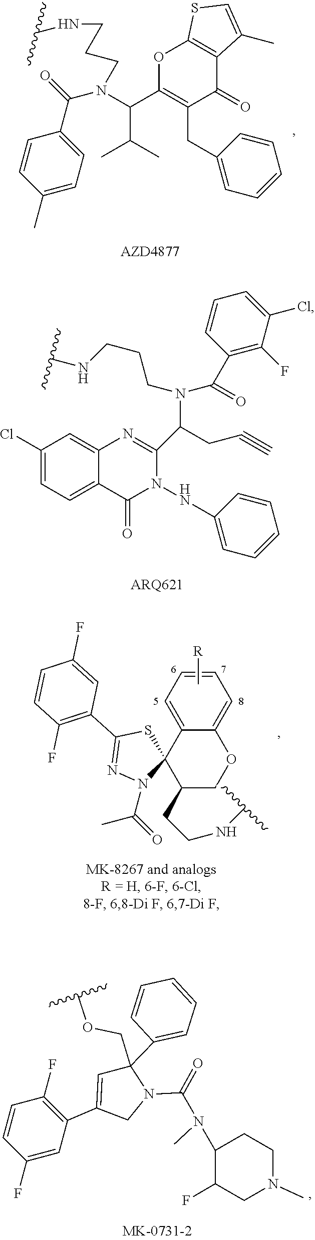

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the linkage site;
11). An antiestrogen: Estrogen blockers or antiestrogens are taken to either block the production of estrogen or block the action of estrogen on receptors. Antiestrogens which are known estrogen deprivation therapy, or endocrine therapy, are a form of hormone therapy that is normally used in the treatment of breast cancer. Estrogen blockers have three modalities: selective estrogen receptor modulators (SERMs) like tamoxifen, selective estrogen receptor degraders like fulvestrant, and aromatase inhibitors like anastrozole and ovariectomy. SERMs act by mimicking estrogen and replacing estrogen on estrogen receptors, blocking estrogen from binding and preventing tumors from using estrogen to grow. Aromatase is the enzyme that catalyzes a key aromatization step in the synthesis of estrogen. It converts the enone ring of androgen precursors such as testosterone, to a phenol, completing the synthesis of estrogen. Accordingly, some structures of conjugatable antiestrogens for the patent are illustrated below:

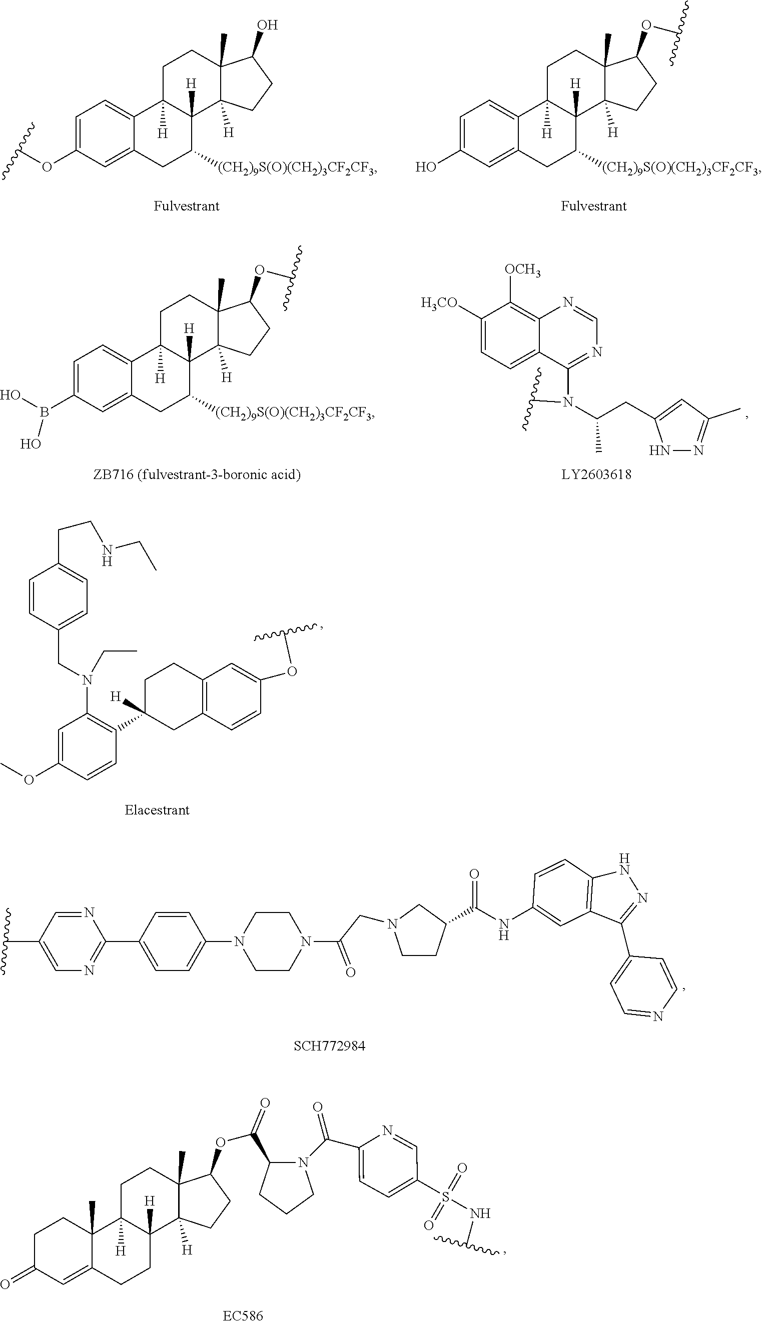

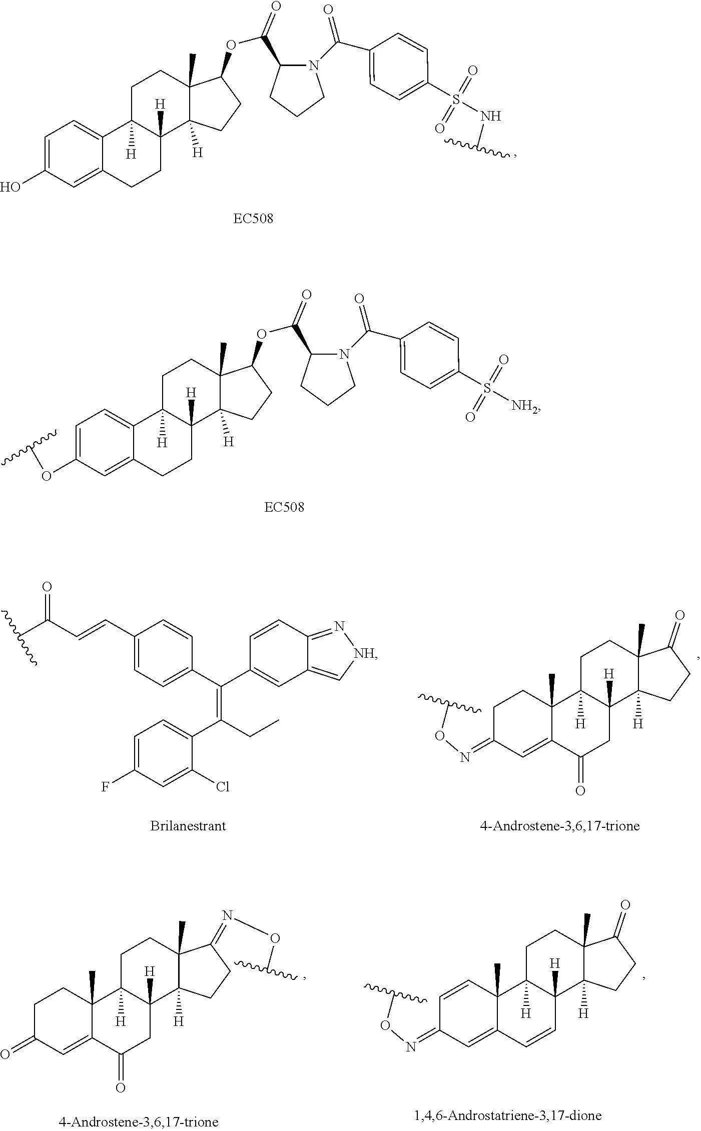

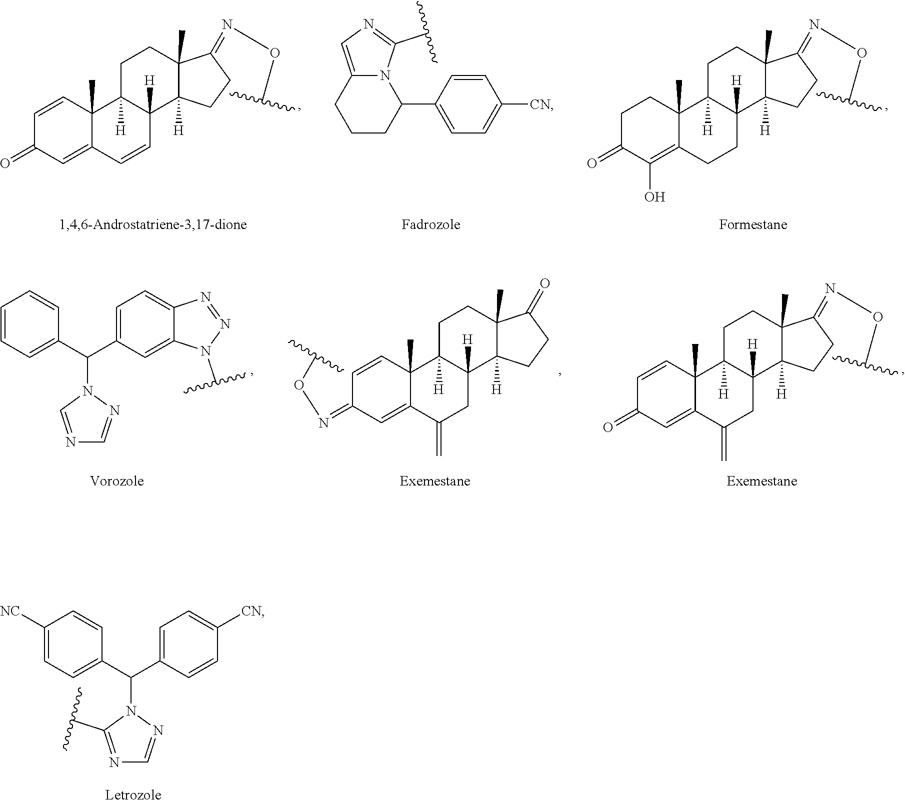

or hydrated salts; or the polymorphic crystalline structures of these compounds; or the optical isomers, racemates, diastereomers or enantiomers; wherein [symbol] is the linkage site;
13). A Proteasome inhibitor (PI): Proteasome is a multi-subunit protein degradation machine, which plays a necessary role not only in cell survival, DNA repair and the proliferation of malignant cells, but also in normal cellular functions, and in the degradation of misfolded or mutated proteins. Proteasomes degrade ubiquitinated proteins or substrates through the ubiquitin-proteasome pathway, a pathway that is utilized in multiple myeloma due to the high protein turnover with immunoglobulin production. Proteasome inhibitors exploit dependence on this pathway, halting protein degradation that ultimately results in apoptosis and cell death. Three proteasome inhibitors, bortezomib, carfilzomib, and ixazomib are approved by the FDA for the treatment of multiple myeloma and many other agents and combinations currently under investigation. The proteasome inhibitors have changed the management of hematologic malignancies and dramatically improved outcomes for patients with multiple myeloma and mantle cell lymphoma. Some of conjugatable PI structures for the invention are illustrated below:

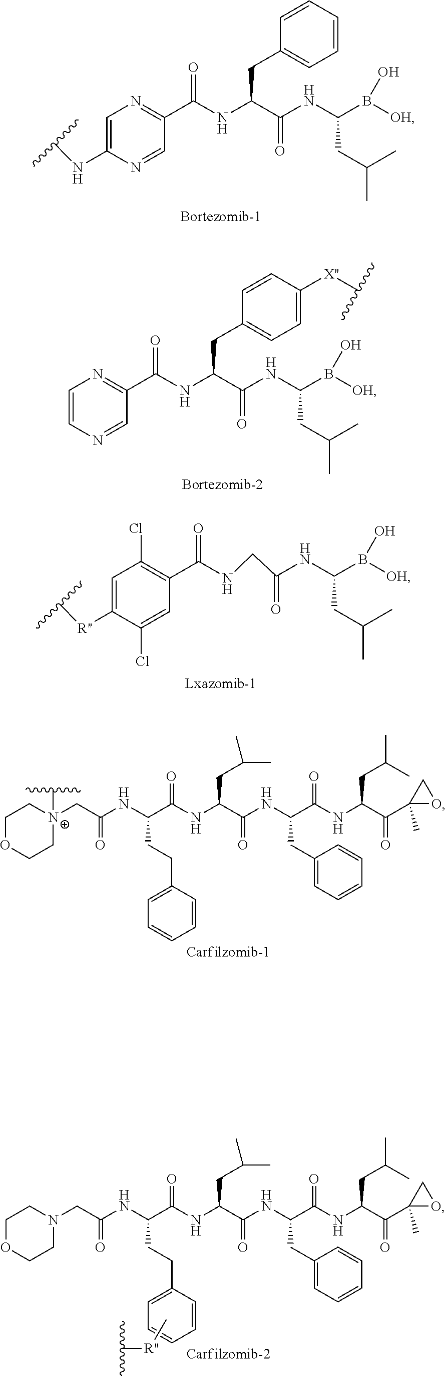

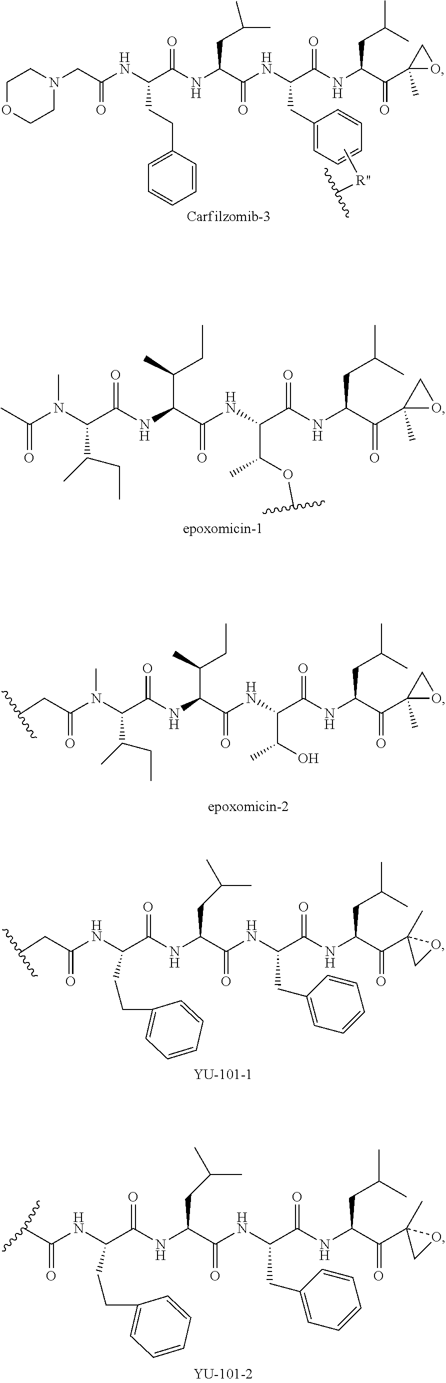

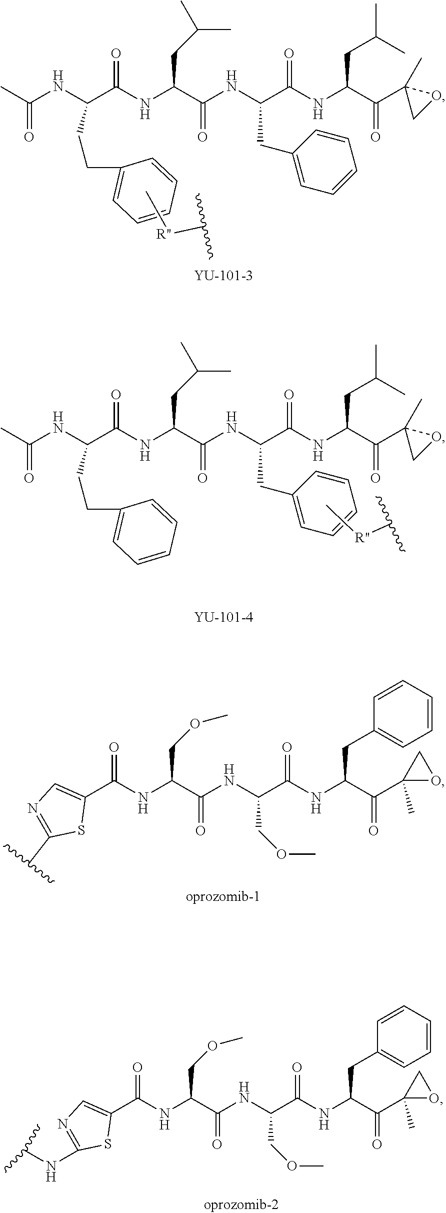

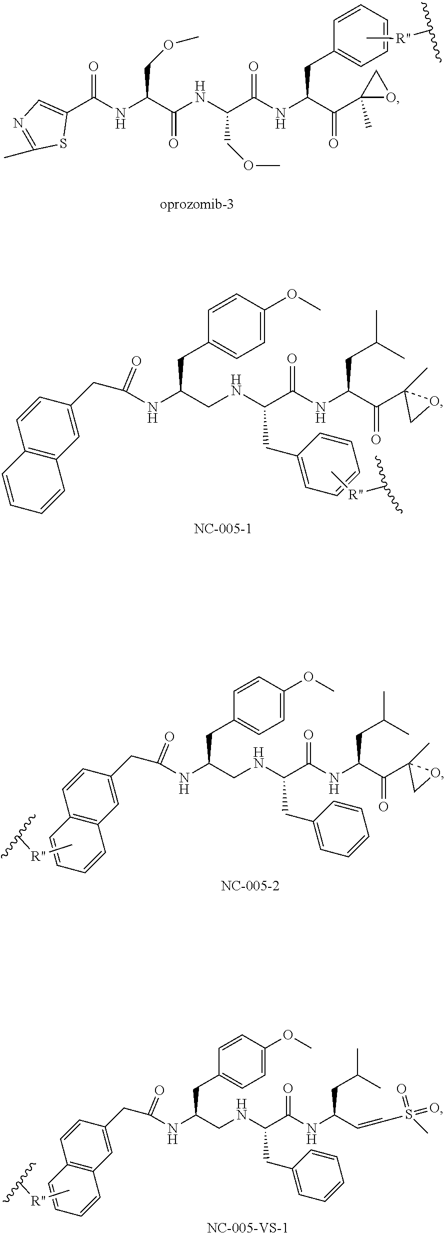

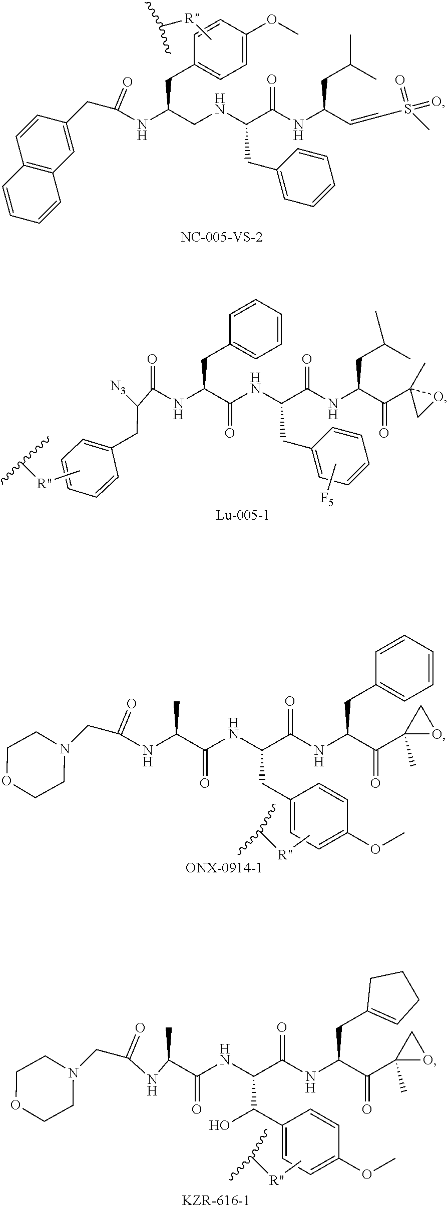

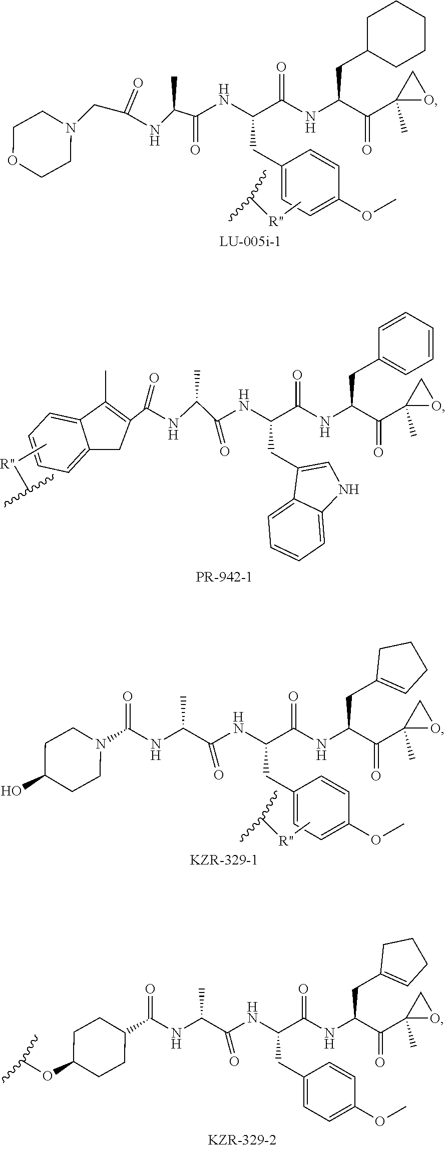

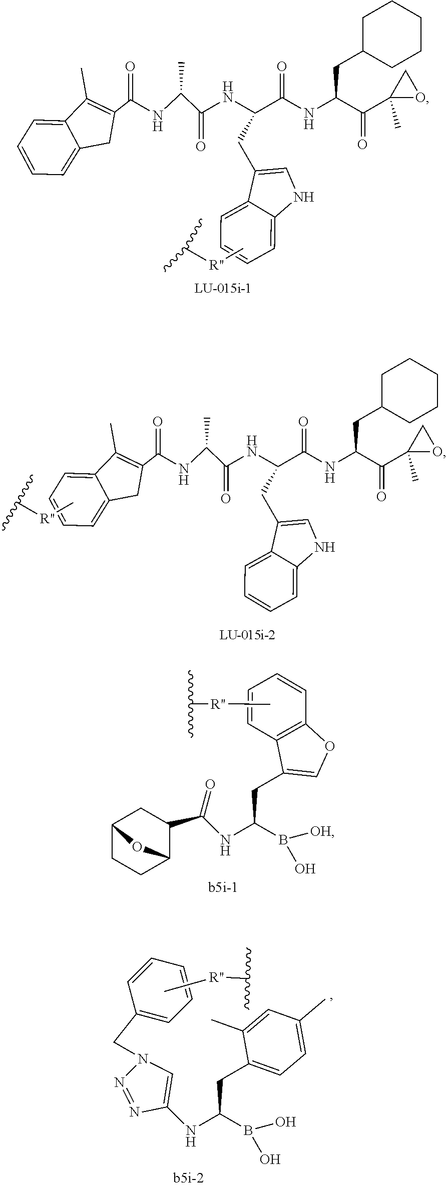

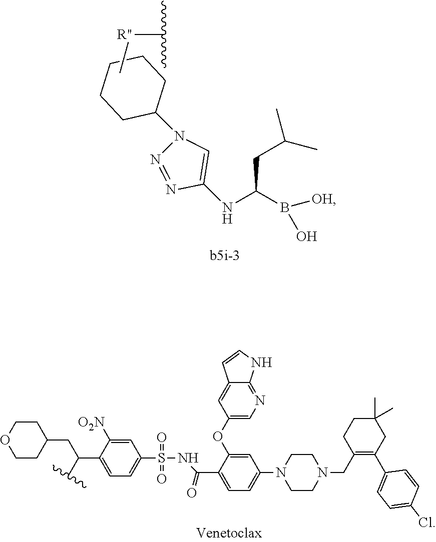

or hydrated salts; or the polymorphic crystalline structures of these compounds; or the optical isomers, racemates, diastereomers or enantiomers; wherein [symbol] is the linkage site; wherein R′ and R″ are NH, O, S, CH₂, CH₂CH₂, C(O), C(O)NH, C(O)O, NHC(O)O—, NHNH, C(O)NHNH respectively; 14). A Toll-like receptor (TLR) Agonist/Antagonist: Toll-like receptors (TLRs) are a large family of proteins that are expressed in immune cells and various tumor cells. TLRs serve as a bridge between innate and adaptive immunity and thus play dual regulatory roles in cell physiopathology. Dysregulation of TLR signaling contributes to numerous pathological conditions, including chronic inflammation, sepsis, cancers, asthma, neuropathic pain, and autoimmune diseases such as lupus, scleroderma, and rheumatoid arthritis (Wang, Y., et al, Acc. Chem. Res., 2020, 53(5), 1046-55). In addition to immune function, TLRs have dual regulatory effects on tumor immunity by activating nuclear factor K—B signaling pathways, which induce tumor immune evasion or enhance the antitumor immune response. Therefore, TLRs have become a popular target for cancer prevention and treatment, and TLR agonists and antagonists offer considerable potential for drug development. TLR agonists also have the potential to convert “cold tumors” into “hot tumors” making TLRs in combination with immune checkpoint inhibitors, potential targets for cancer therapies (Roffo, C., et al. npj Precision Oncology 2023; 7:26). Various agonists/Antagonists of TLRs have been under investigation for cancer treatments (Duan, T, et al, Front Immunol, 2022, 13, 812774) such as, TLR1/2: Bacterial lipoprotein, Pam3CSK4; TLR2/TLR4: OM-174; TLR3: Poly I: C, poly-ICLC (Hiltonol), poly-IC12U (Ampligen); TLR4: TAK-242 (resatorvid), Eritoran, MPLA, GLA-SE; TLR5: Mobilan, Entolimod; TLR7: Imiquimod; TLR7/TLR8: MEDI9197, Resiquimod; TLR7/TLR9: Chloroquine; TLR9: CpG, MGN1703, SD-101; Imiquimod is a topical TLR7 agonist, approved by the FDA for antiviral and skin cancer treatments. Other TLR adjuvants are used in several vaccines including Nu Thrax, Heplisav, T-VEC, and Cervarix. Many TLR agonists, in particular, TLR7 and TLR8, are currently in development as both monotherapy and in combination with immune checkpoint inhibitors, A TLR7 agonist is preferably an imidazo-quinoline, an imidazoquinoline amine, a thiazoquinoline, an aminoquinoline, an aminoquinazoline, a pyrido[3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaph-thyridine, a guanosine analog, an adenosine analog, a thymidine homopolymer, ssRNA or modified oligonucleotides, CpG-A, PolyG10, and PolyG3. A TLR8 agonist is prferably a benzazepine, an imidazoquinoline, a thiazoloquinoline, an aminoquinoline, an aminoquinazoline, a pyrido[3,2-d]pyrimidine-2,4-diamine, pyrimidine-2,4-diamine, 2-aminoimidazole, 1-alkyl-1H-benzimidazol-2-amine, tetrahydropyridopyrimidine. In some embodiments, a TLR agonist is a nonnaturally occurring compound. Examples of TLR7 modulators include GS-9620, GSK2245035, imiquimod, resiquimod, DSR-6434, DSP-3025, IM0-4200, MCT-465, MEDI9197, 3M-051, SB-9922, 3M-052, Limtop, TMX-30X, TMX-202, RG-7863, RG-7795, TQ-A₃₃₃₄, SHR2150, RO7119929, DSP-0509, BNT411, APR003, BDB001, BDC-1001, CV8102, TransCon TLR7/8, MBS-8, BDB-018, Imiquimod (UGN-201), Lefitolimod (MGN1703), Tilsotolimod (IMO-2125), CMP-001, SD-101, TAC-001 (Roffo, C., et al. npj Precision Oncology 2023; 7:26), and some compounds disclosed in US20160168164, US20150299194, US20110098248, US20100143301, and US20090047249. Examples of TLR8 agonists include motolimod, resiquimod, 3M-051, 3M-052, MCT-465, IM0-4200, VTX-763, VTX-1463. In some embodiments, a TLR8 agonist can be any of the compounds described WO2018/170179, WO2020/056198 and WO2020056194. Other TLR7 and TLR8 agonists are disclosed in, for example, WO2007024612, US20080234251, US20080306050, US20100029585, WO2011022508, WO2011022509, US20110092485, US20110118235, US20120082658, UD20120219615, WO2012045090, WO2012097173, WO2012097177, US20130251673, US20140045849, US20140066432, US20140088085, US20140275167, US20140073642, WO2014056953, WO2014076221, WO2014128189, US20140350031, WO2014023813, WO2016142250, US20160008374, US20160194350, US20160289229, US20170131421, WO2017046112, WO2017079283, WO2017216054, WO2017190669, WO2017202704, WO2017202703, WO20170071944, US20180086755, WO2018198091, WO2018196823, WO2018203613, WO2018132539, WO2018049089, WO2018026620, WO2019028301, WO2019028302, WO2019036023, WO2019035968, WO2019035971, WO2019035970, WO2019036031, WO2019040491, WO2019099336, WO2019118799, WO2019125977, WO2019126082, WO2019126081, WO2019126083, WO2019116113, WO2019126253, WO2019126242, WO2019225920, WO2020023680, WO2020051356, WO2020074006, WO2020086503, WO2020086505, WO2020162705, WO2020227337, WO2020227484, WO2021067261, WO2021067326, WO2021064614, WO2021067657, WO2021086699, WO2021087181, WO2021099402, WO2021177679, WO2021232099, WO2022031057, WO2022047279, WO2022114808, WO2022213153, WO2023079428, WO2023198195, WO2024077351, WO2024095964.

In some embodiments, an immune modulatory agent is a STING agonist. Examples of STING agonists include, for example, those disclosed in WO2015077354, WO2016079899, WO2016096577, WO2017100305, WO2017223422, WO2018200812, WO2019069275, WO2019129880, WO2019134707, WO2019072048, WO2019150310, WO2020023361, WO2020038387, WO2020042995, WO2020050406, WO2020049534, WO2020075790, WO2020074004, WO2020075790, WO2020089815, WO2020092617, WO2020117739, WO2020135715, WO2020151682, WO2020156363, WO2020163415, WO2020205323, WO2020227159, WO2021005541, WO2021016204, WO2021013250, WO2021062060, WO2021076666, WO2021079302, WO2021086889, WO2021083060, WO2021177438, WO2021200824, WO2021206158, WO2021206160, WO2021216572, WO2021216467, WO2021216698, WO2021228284, WO2021232019, WO2021231350, WO2022002077, WO2022055929, WO2022079175, WO2022097634, WO2022107027, WO2022116369, WO2022150779, WO2022228469, WO2022242737, WO2022248353, WO2022272039, WO2023284719, WO2023004440, WO2023011076, WO2023019259, WO2023020993, WO2023025256, WO2023033506, WO2023039363, WO2023056468, WO2023059544, WO2023066872, WO2023103850, WO2023172906, WO2023178038, WO2023211930, WO2023239675, WO2024006804, WO2024040027, WO2024040169, WO2024048490, WO2024081811, WO2024100449, WO2024100452, WO2024137619, WO2024151778, WO2024153127, WO2024174863, WO2024182569, and In some embodiments, an immune modulatory agent is a retinoic-acid-inducible protein 1 (RIG-I) agonist. Examples of RIG-I agonists include KIN1148, SB-9200, KIN700, KIN600, KIN500, KIN100, KIN101, KIN400 and KIN2000, pUUC-Auk, 3p-hpRNA. Examples of RIG-I agonists include, for example, those disclosed in WO2016179034, WO2016179475, WO2018232257, WO2019246450, WO2020225779, WO2021067480, WO2021097347, WO2022178325, WO2022251406, WO2023049777, WO2023172208, WO2024006362, WO2024151778. Some of conjugatable structures of Toll-like receptor (TLR) Agonists/Antagonists and/or STING agonists for the invention are illustrated below:

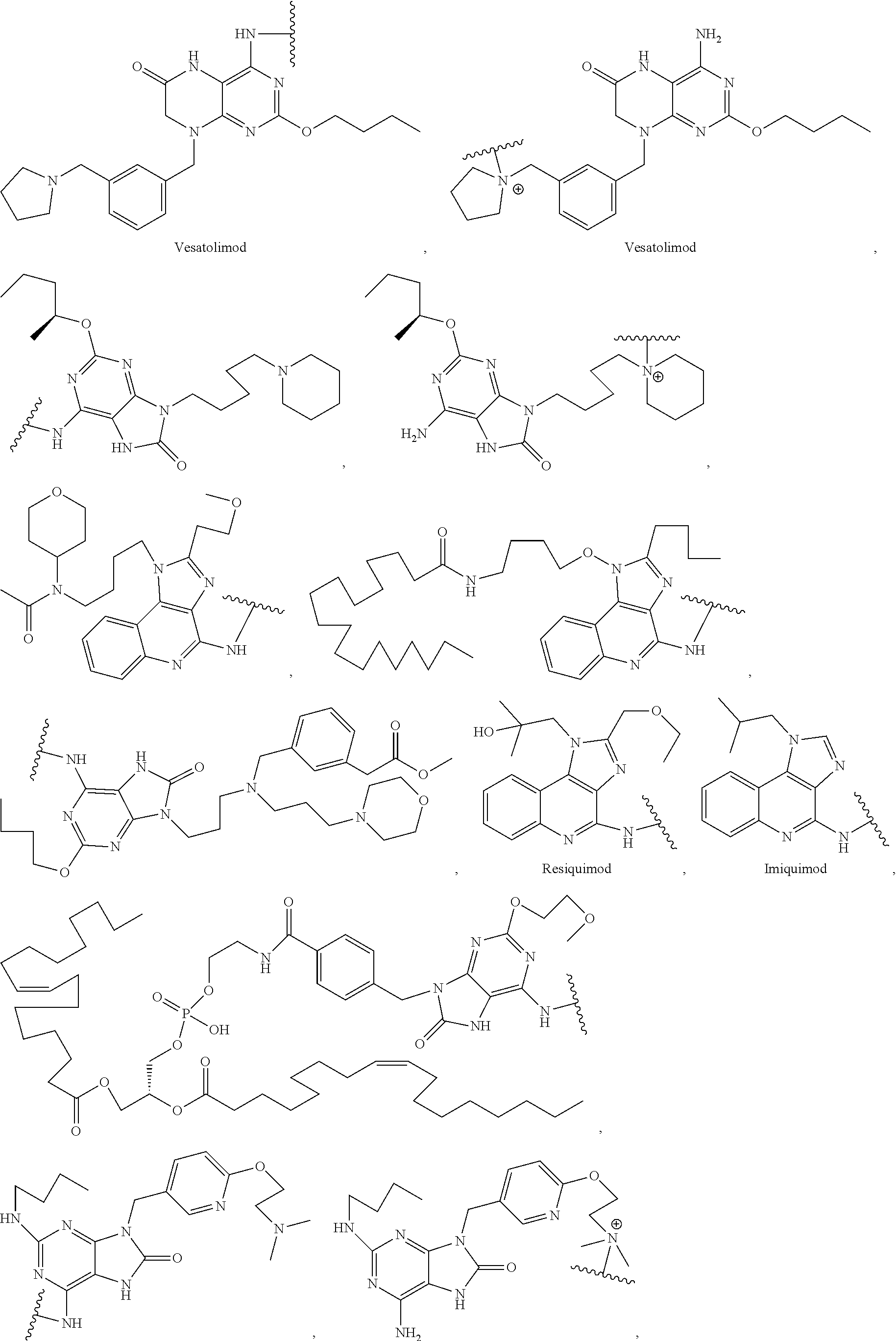

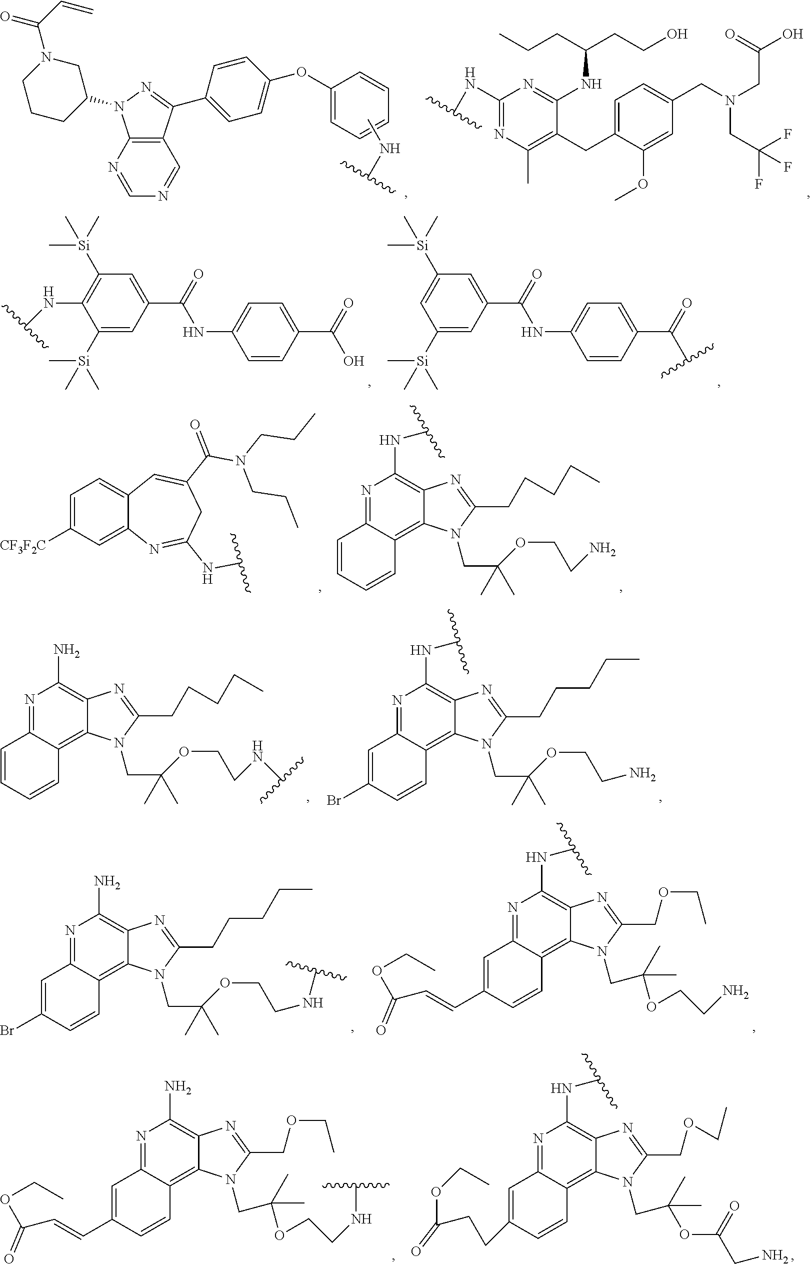

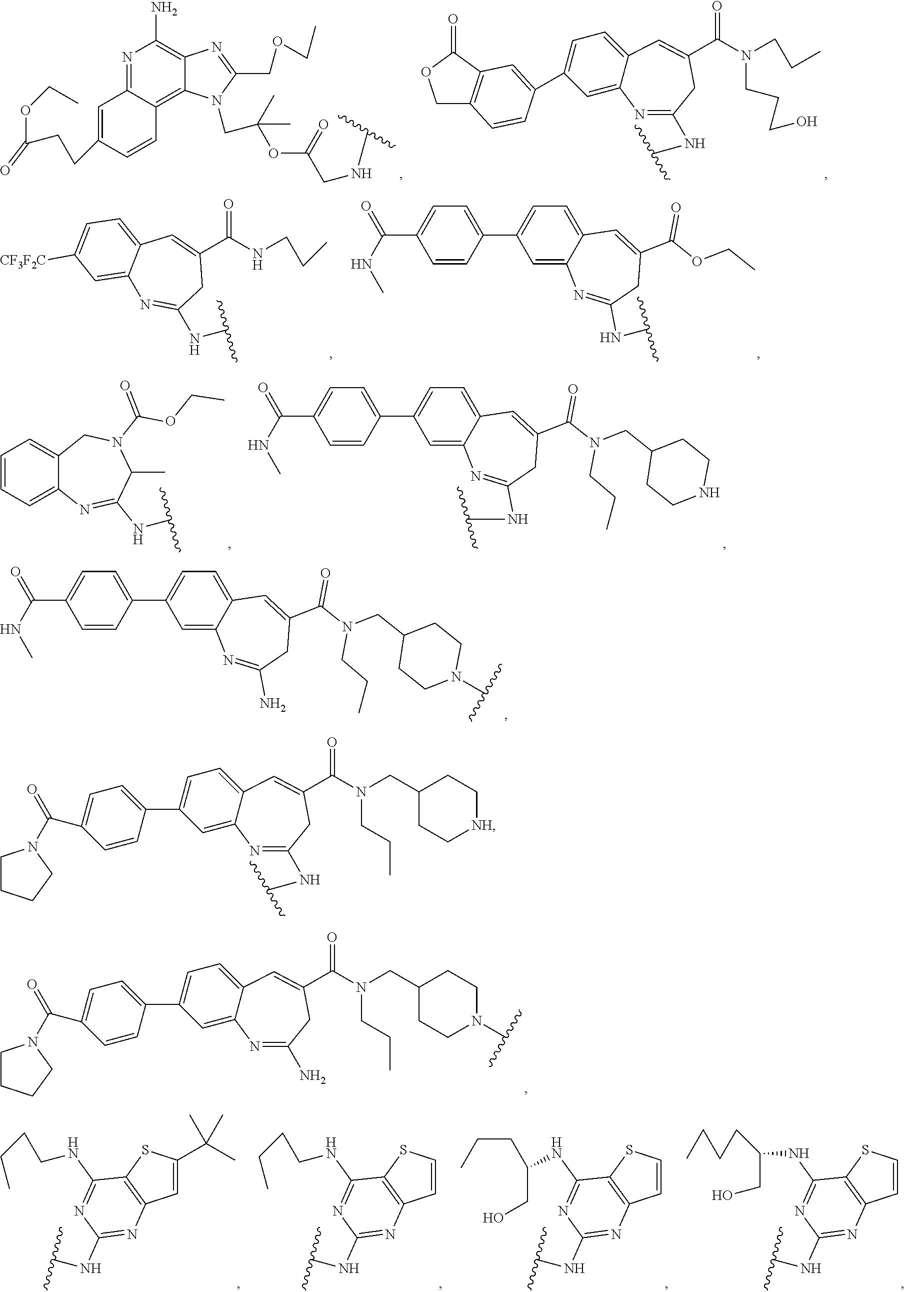

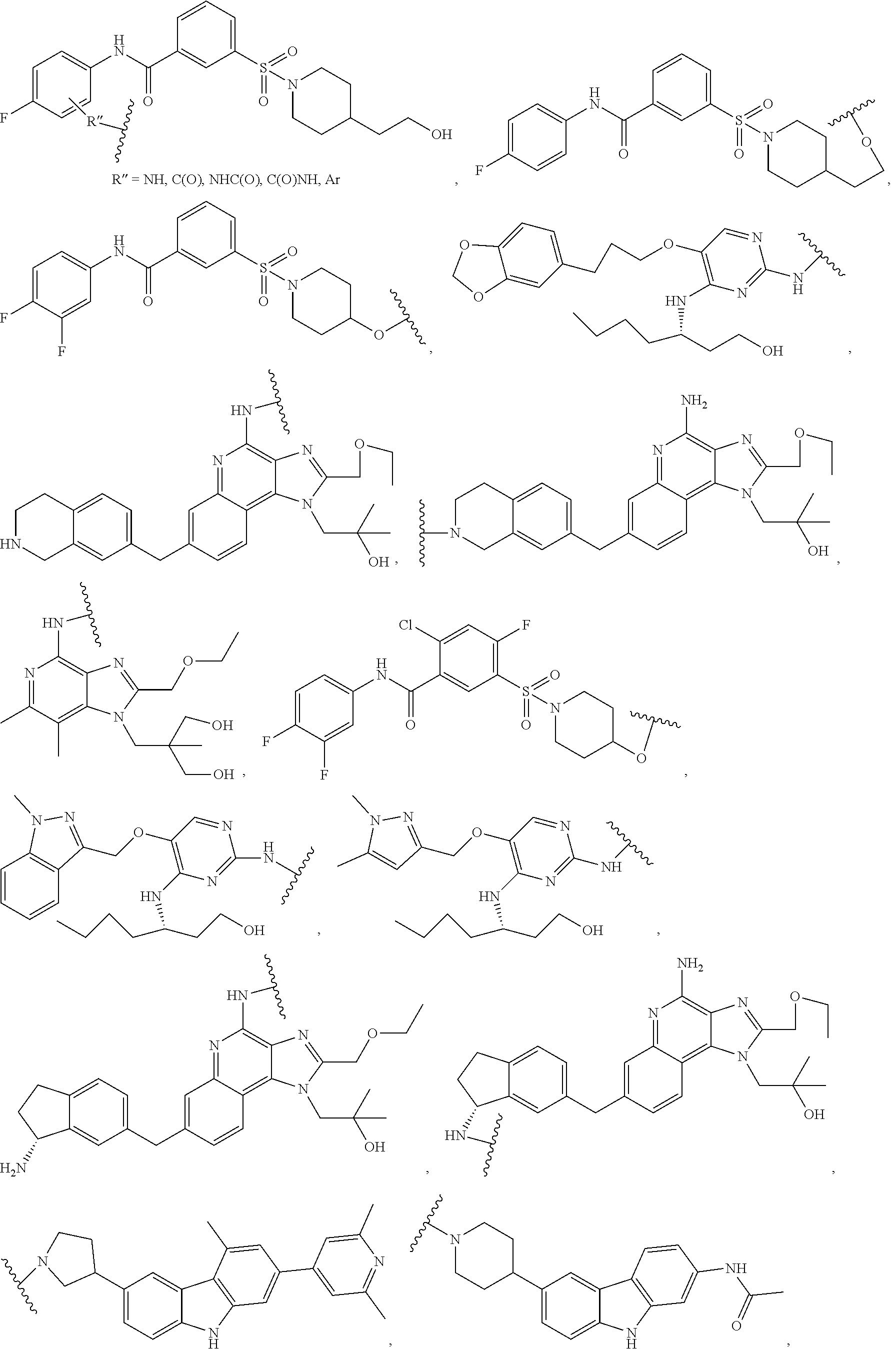

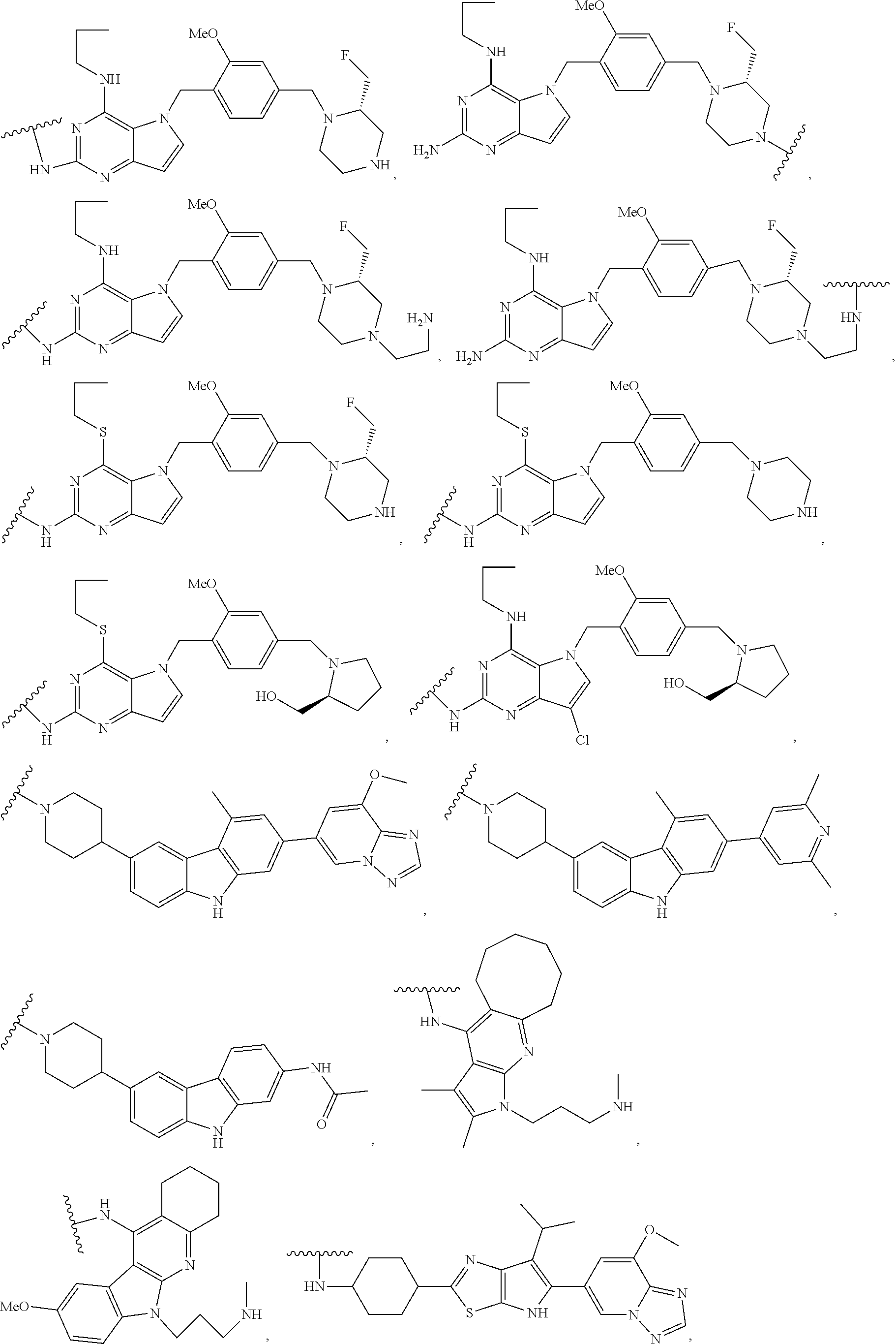

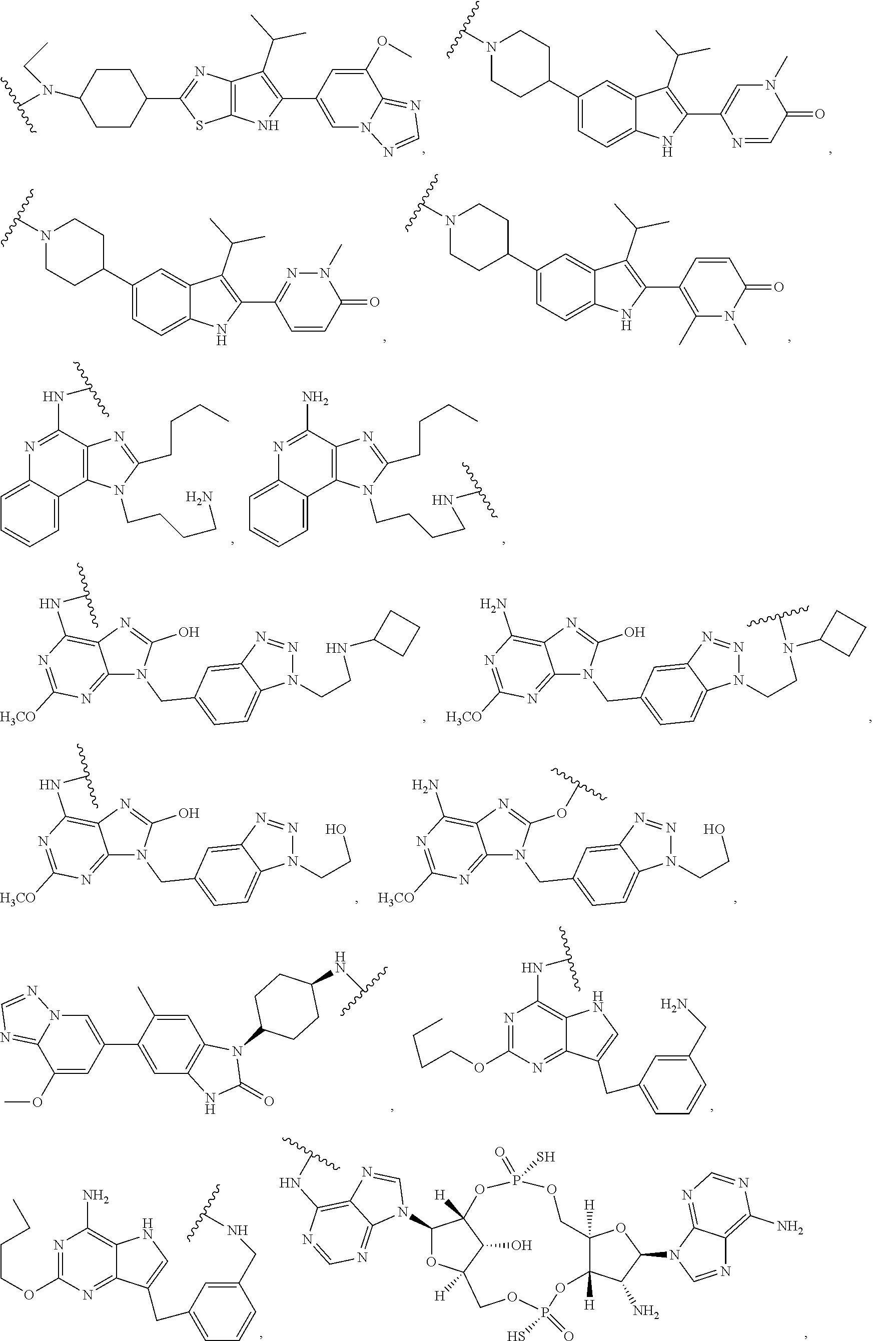

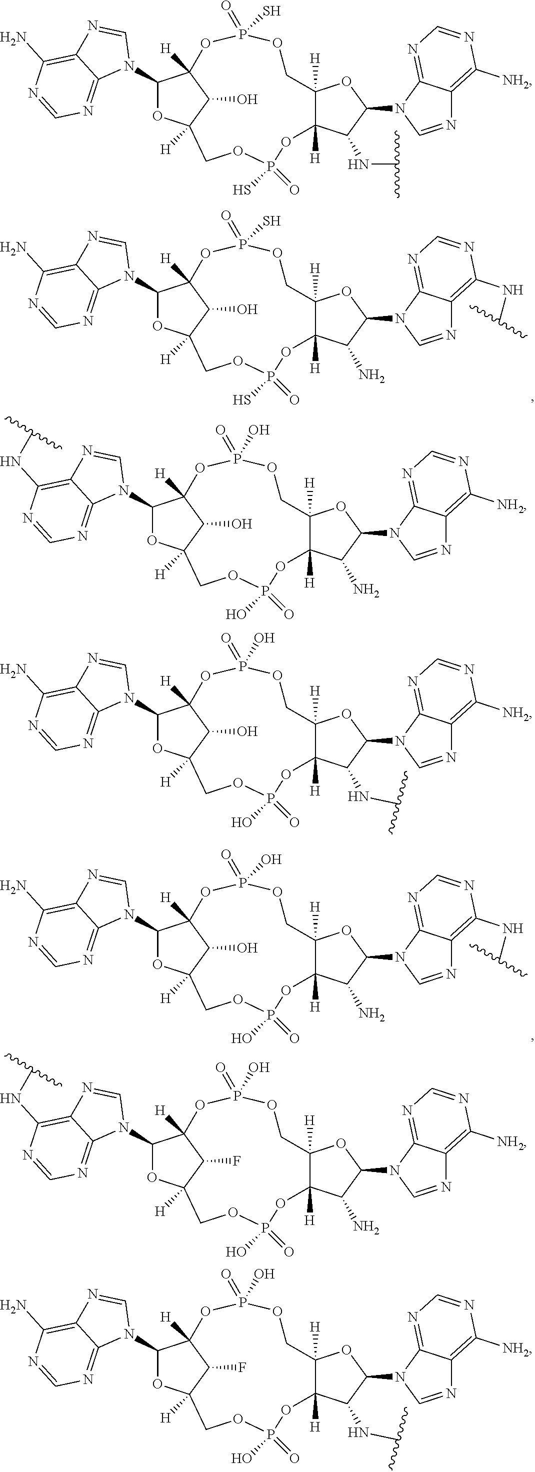

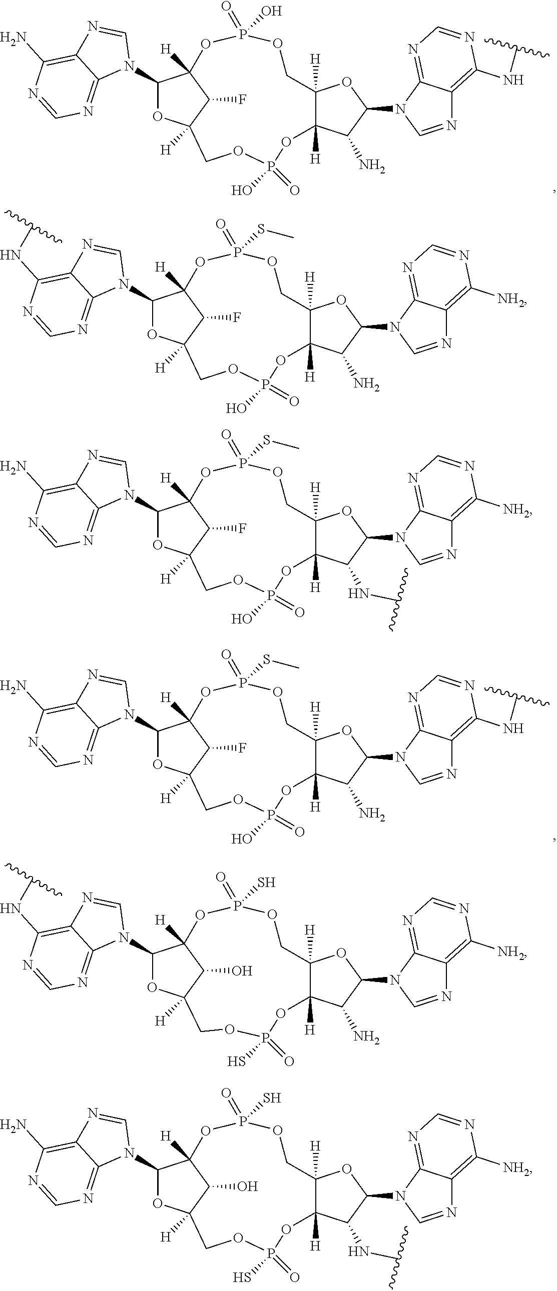

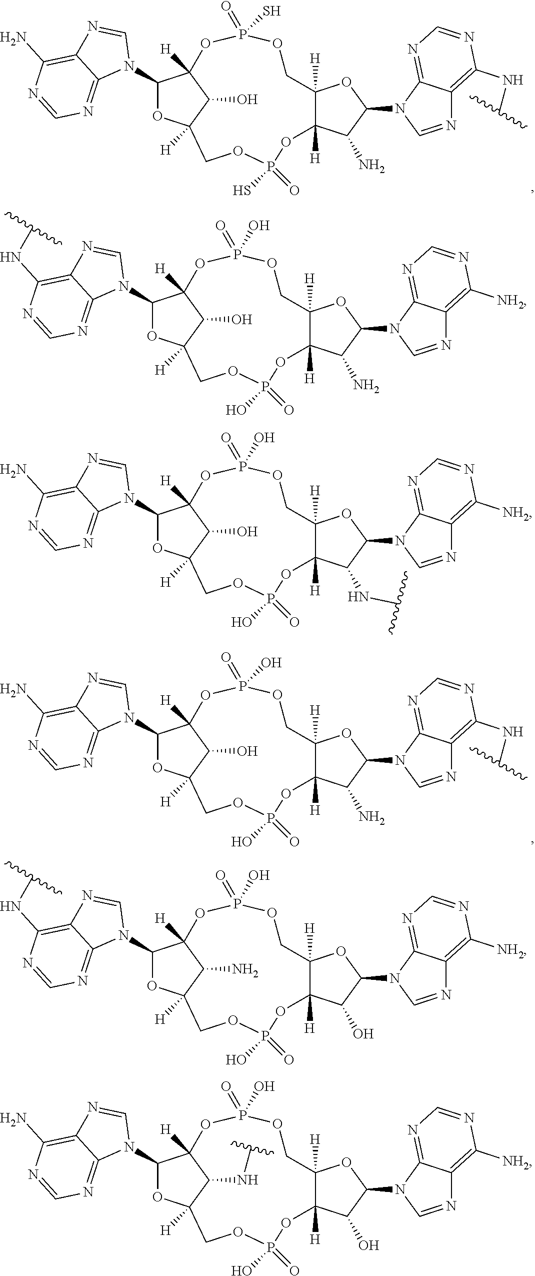

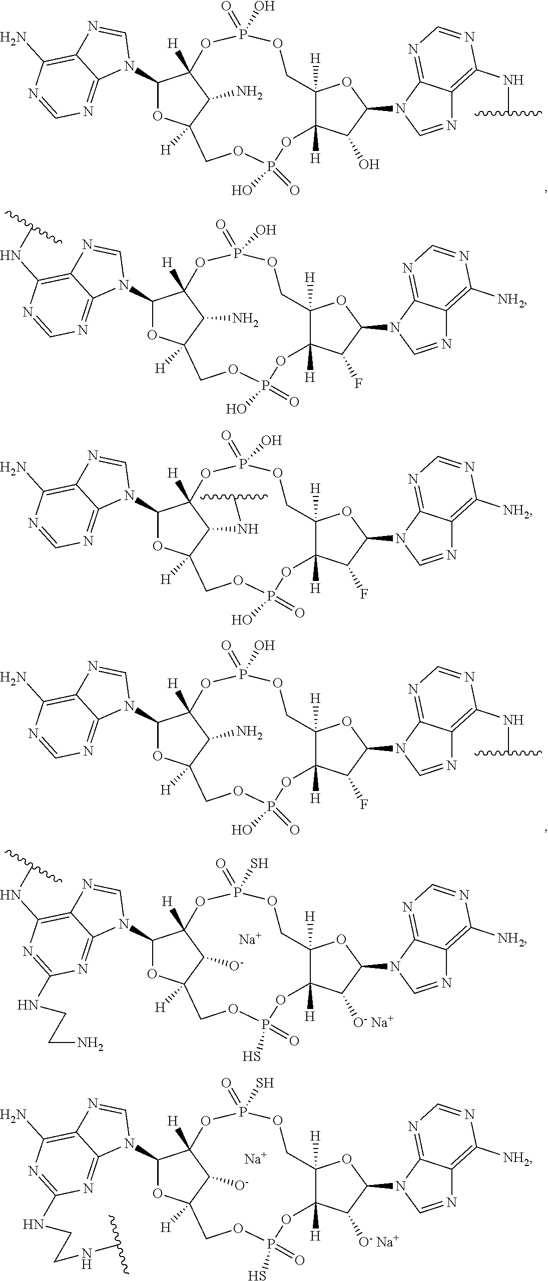

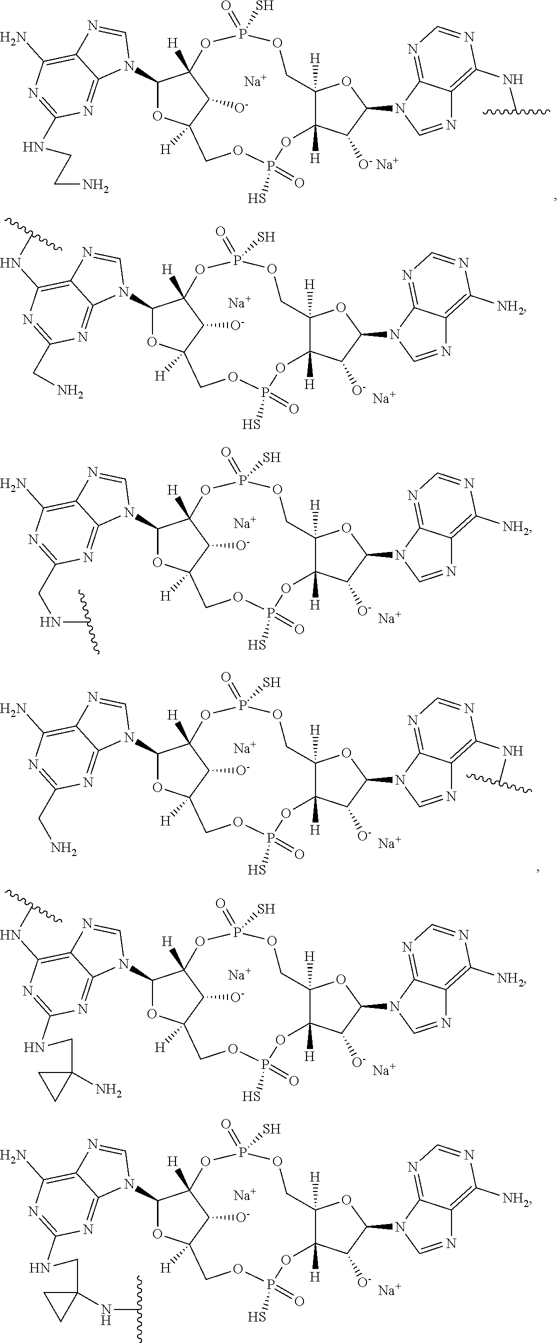

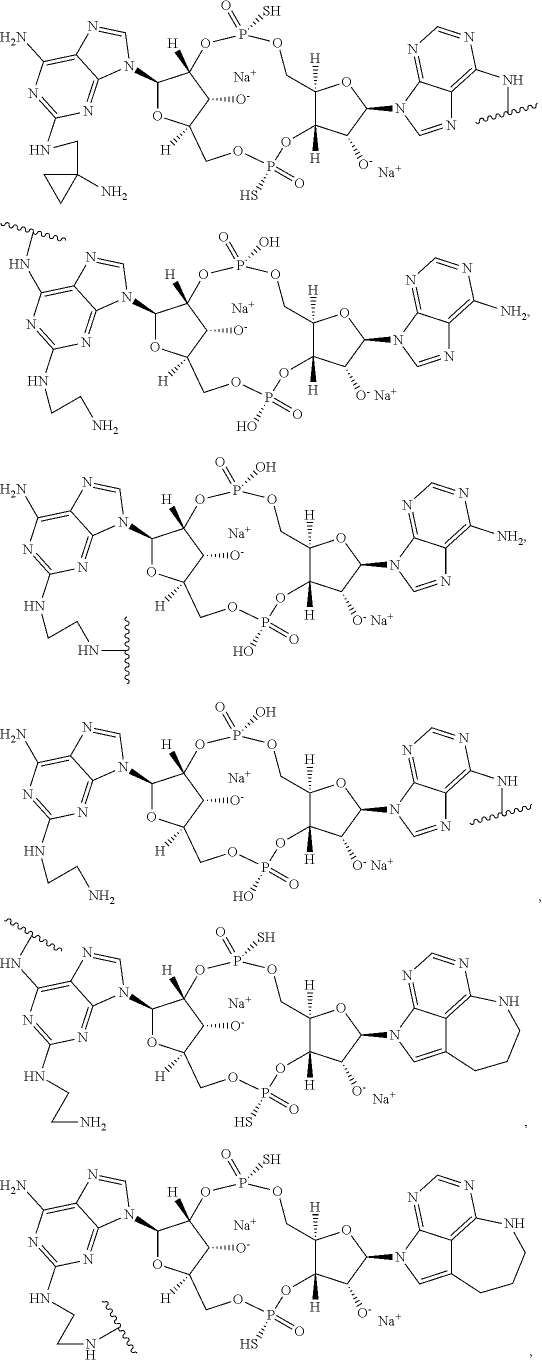

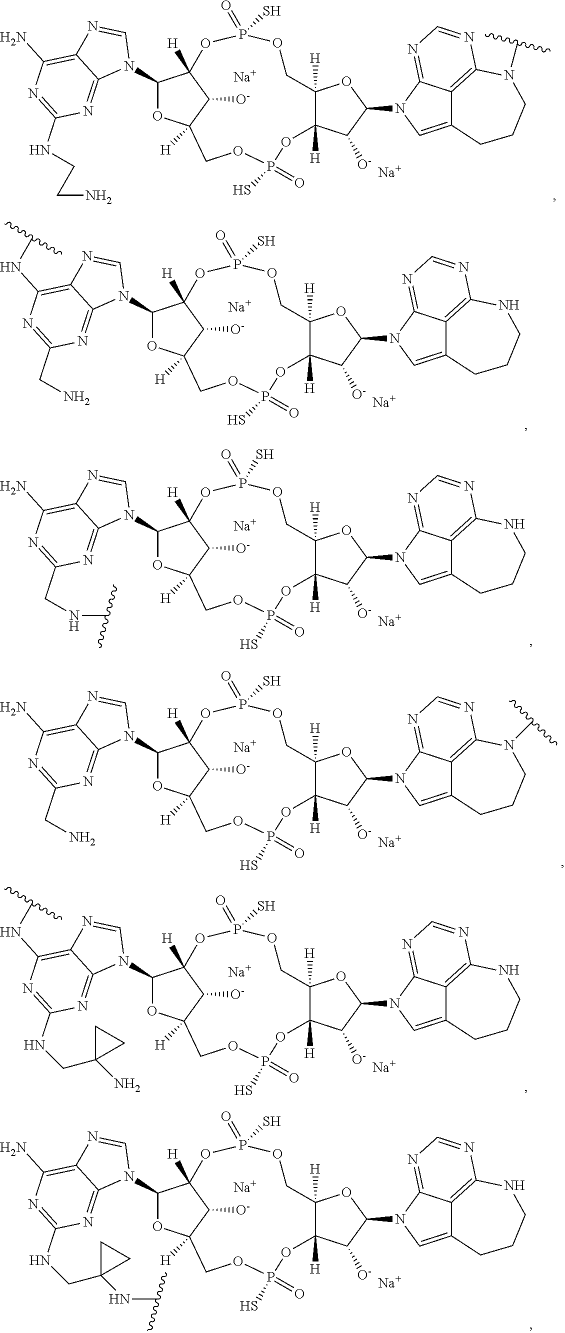

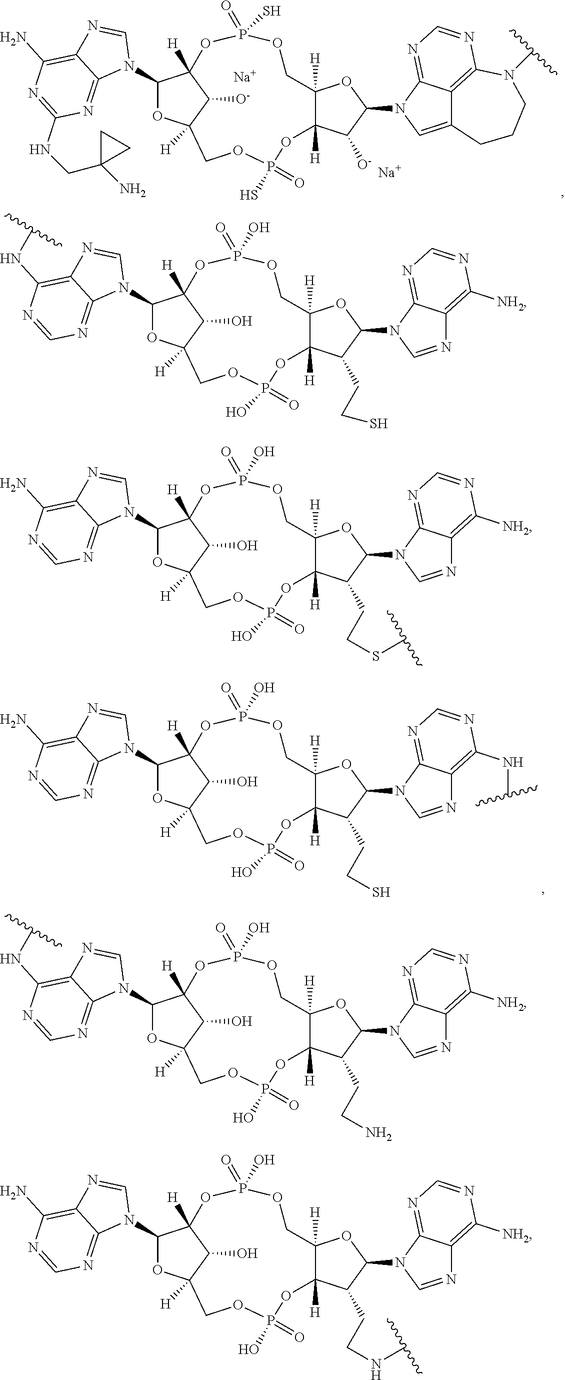

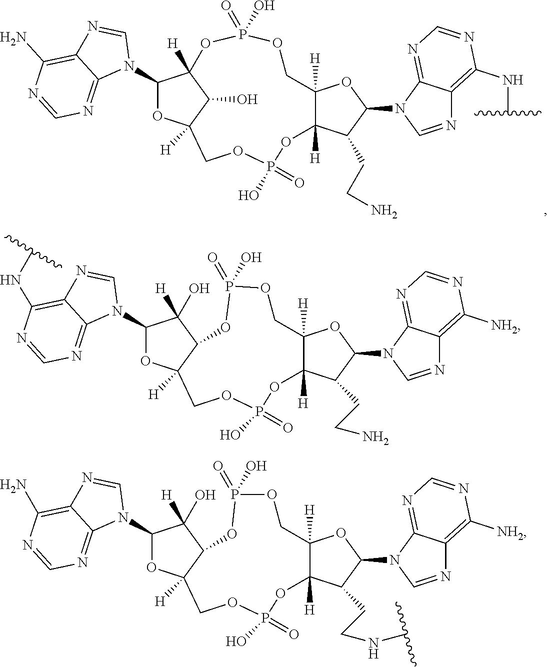

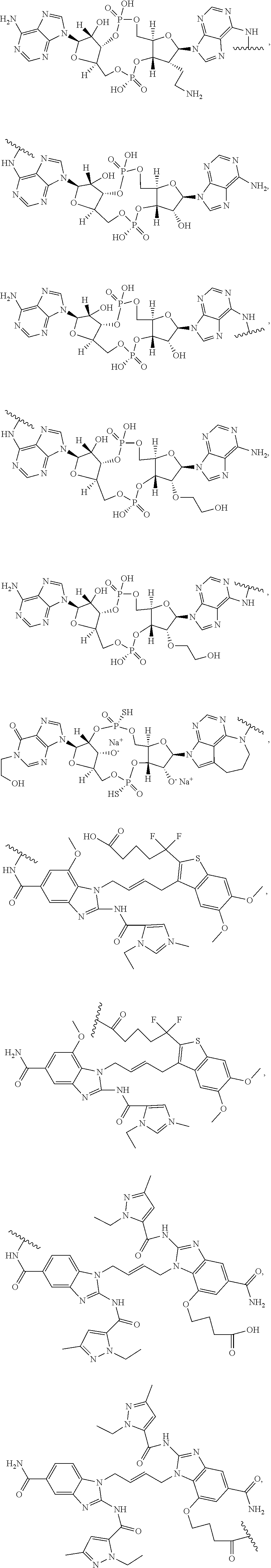

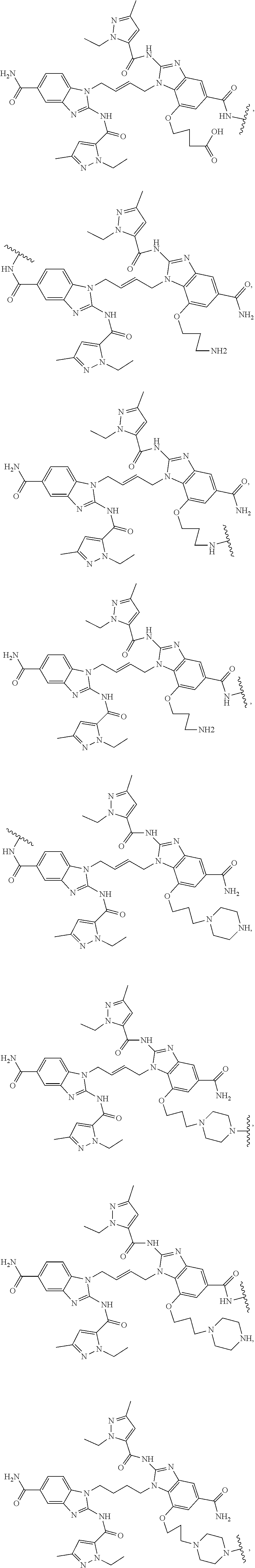

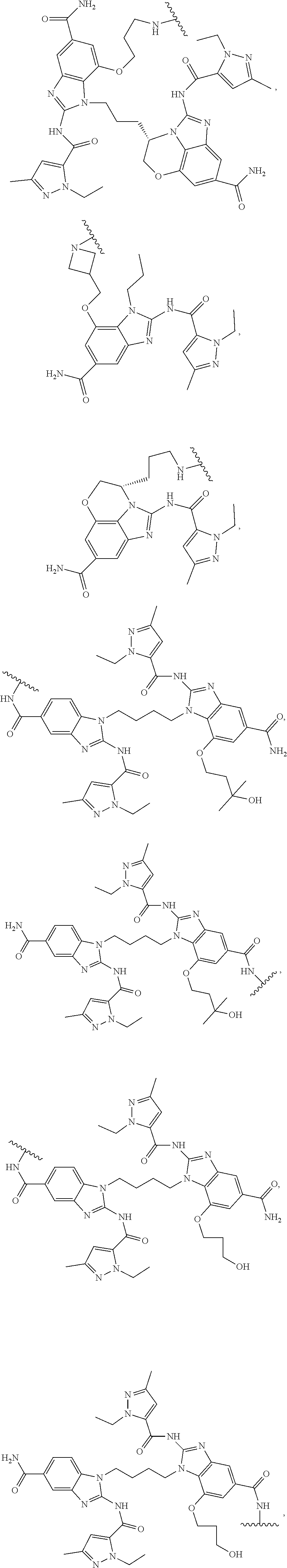

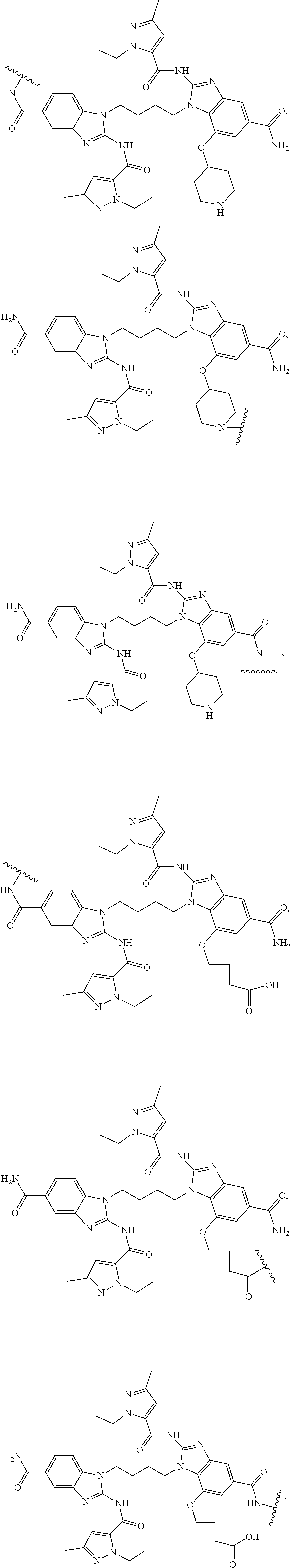

or hydrated salts; or the polymorphic crystalline structures of these compounds; or the optical isomers, racemates, diastereomers or enantiomers; [symbol] is a linkage site that links to L₁ and/or L₂ independently.
15). A proteolysis-targeting chimera (PROTAC) molecule: Targeted protein degradation (TPD) is an emerging therapeutic modality with the potential to tackle disease-causing proteins that have historically been highly challenging to target with conventional small molecules. A major class of molecules that may enable such proteins to be modulated through TPD are known as proteolysis-targeting chimera (PROTAC) protein degraders. These heterobifunctional molecules are capable of specifically degrading target a protein of interest (POI) within cells, thus they have the potential to improve and/or prolong biological activity relative to simple small molecule inhibitors of the same entity. PROTACs generally consist of a binding element that recognizes the target protein, a separate part that binds the E3 ligase, and a spacer that connects the two components. Simultaneous binding of the POI and ligase by the PROTAC induces ubiquitylation of the POI and its subsequent degradation by the ubiquitin-proteasome system (UPS), after which the PROTAC is recycled to target another copy of the POI. Degrader-antibody conjugates (DACs) are a new class of therapeutic agents that have garnered significant interest in recent years. Currently, there are several DACs in preclinical and clinical development. One of the most advanced DACs is ARV-471, which targets the estrogen receptor (ER) in breast cancer cells and is now being evaluated in a clinical trial. Other DACs in development target a variety of cancer-associated proteins, including BCL-2, BRD4, and FLT3. Some of conjugatable structures of PROTAC payloads for the invention are illustrated below:

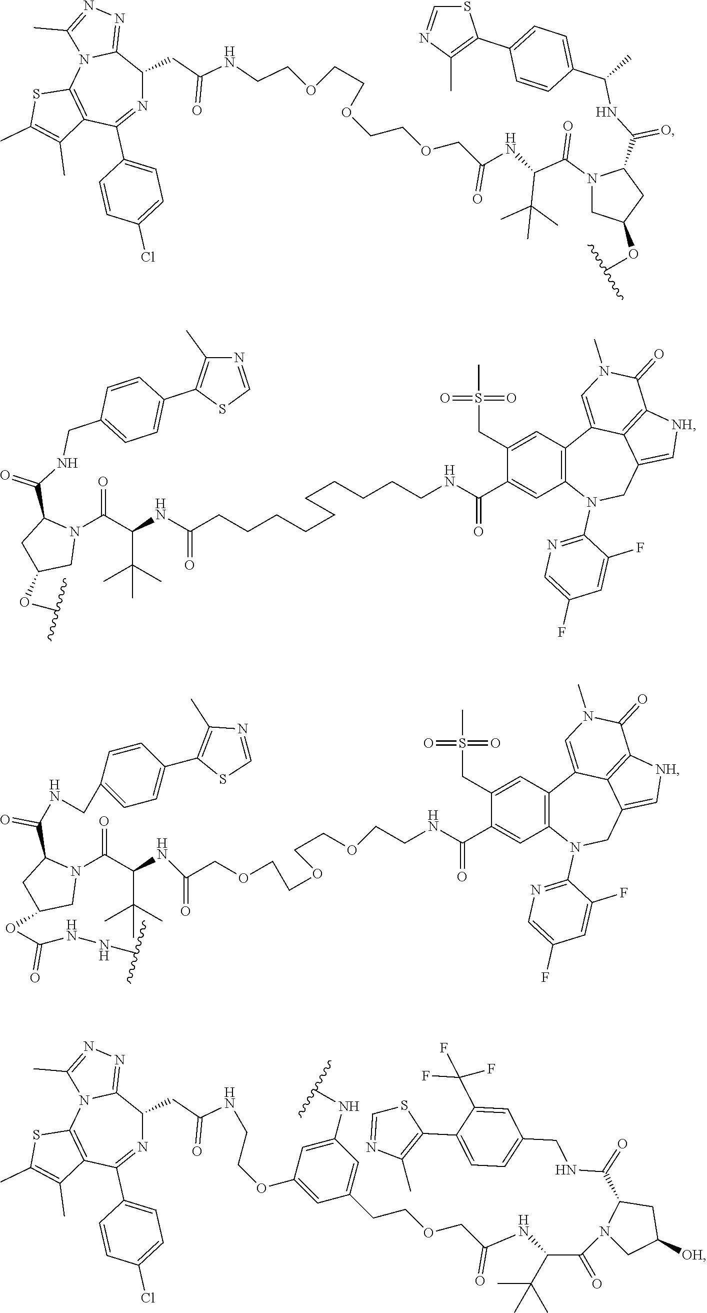

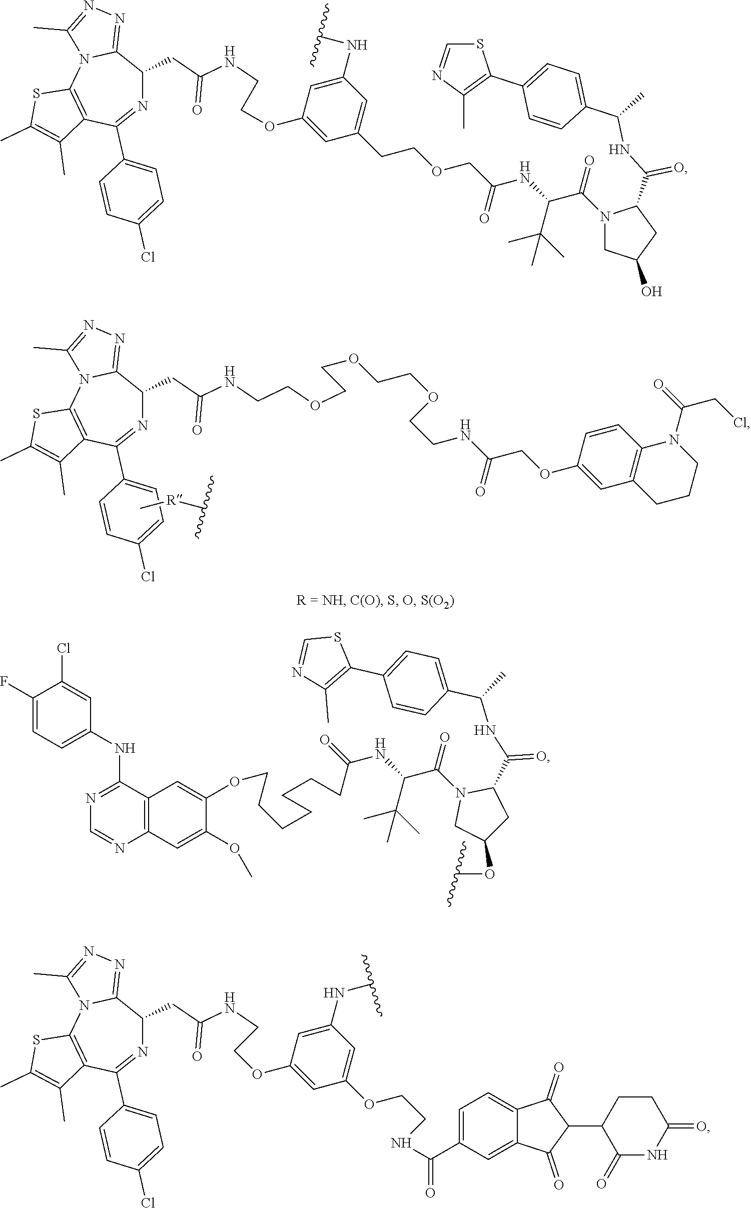

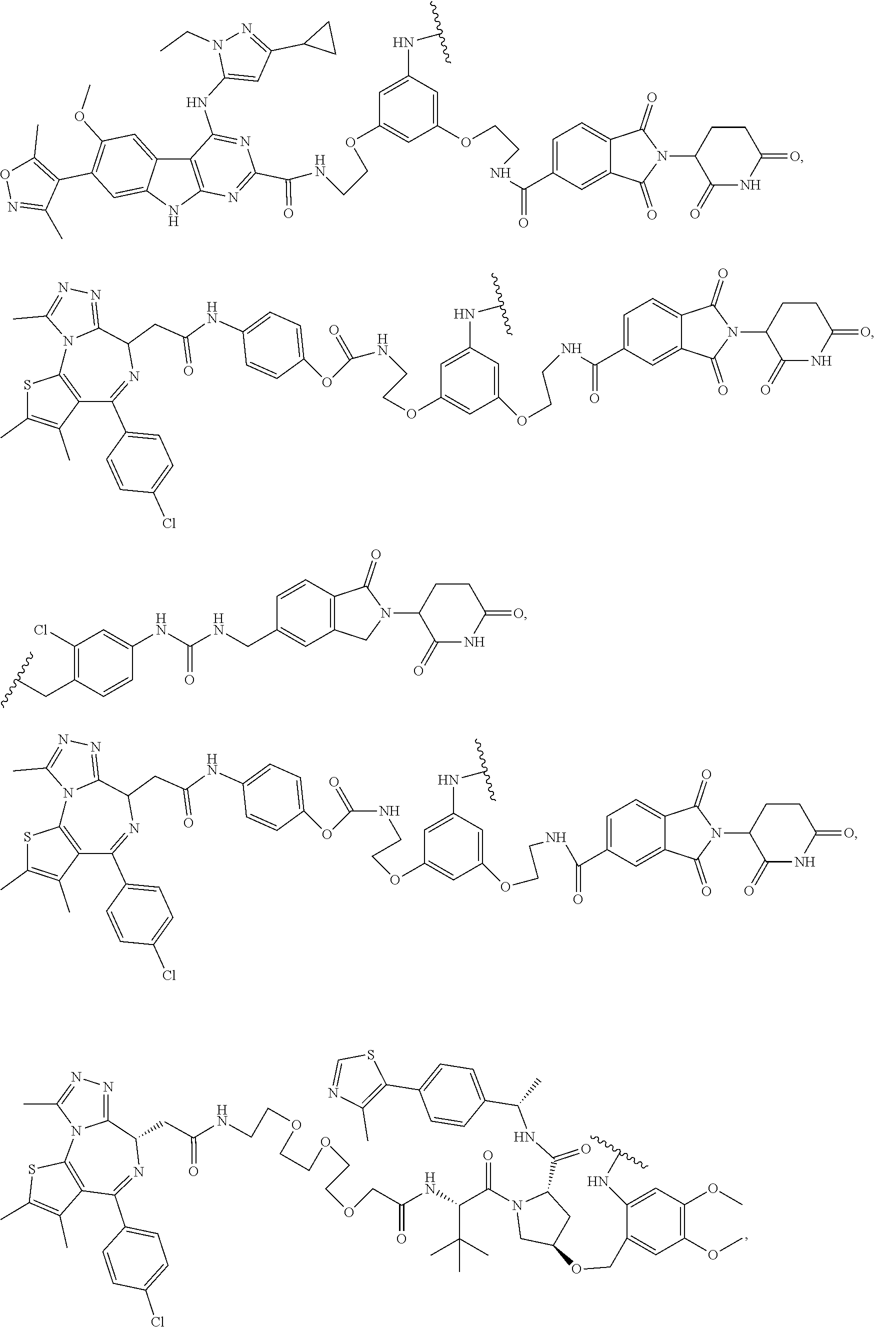

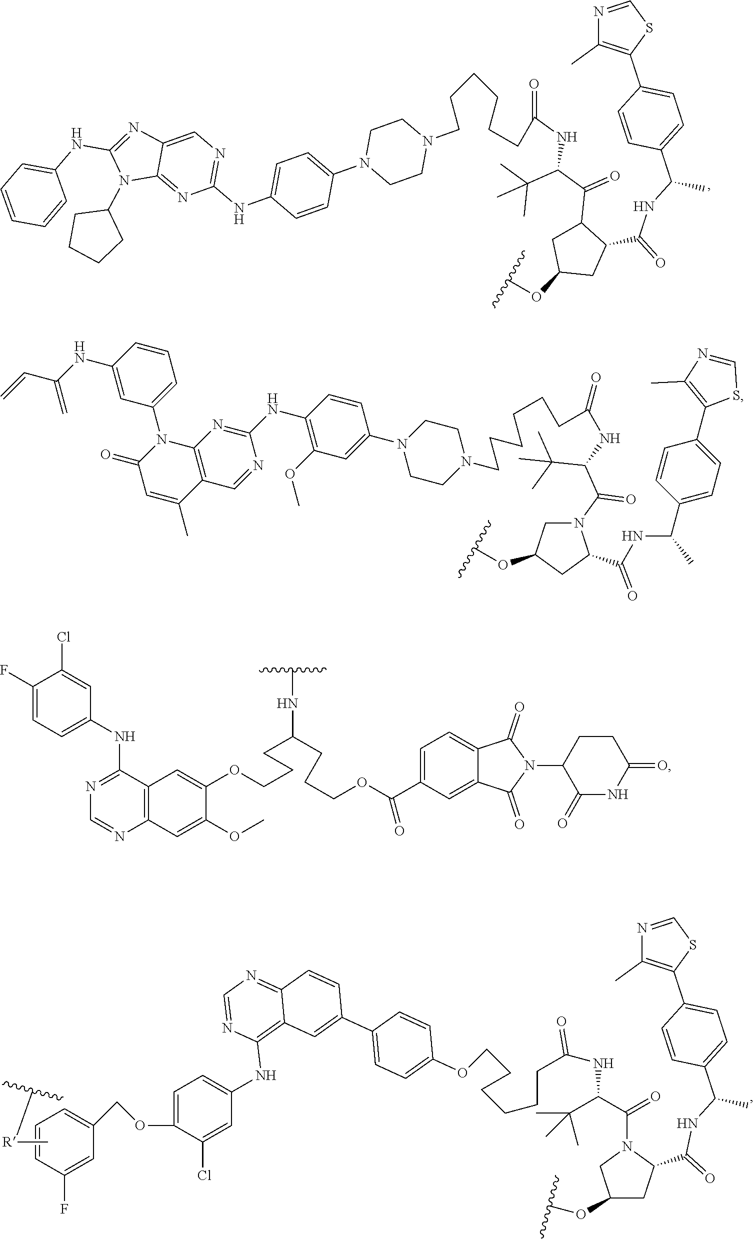

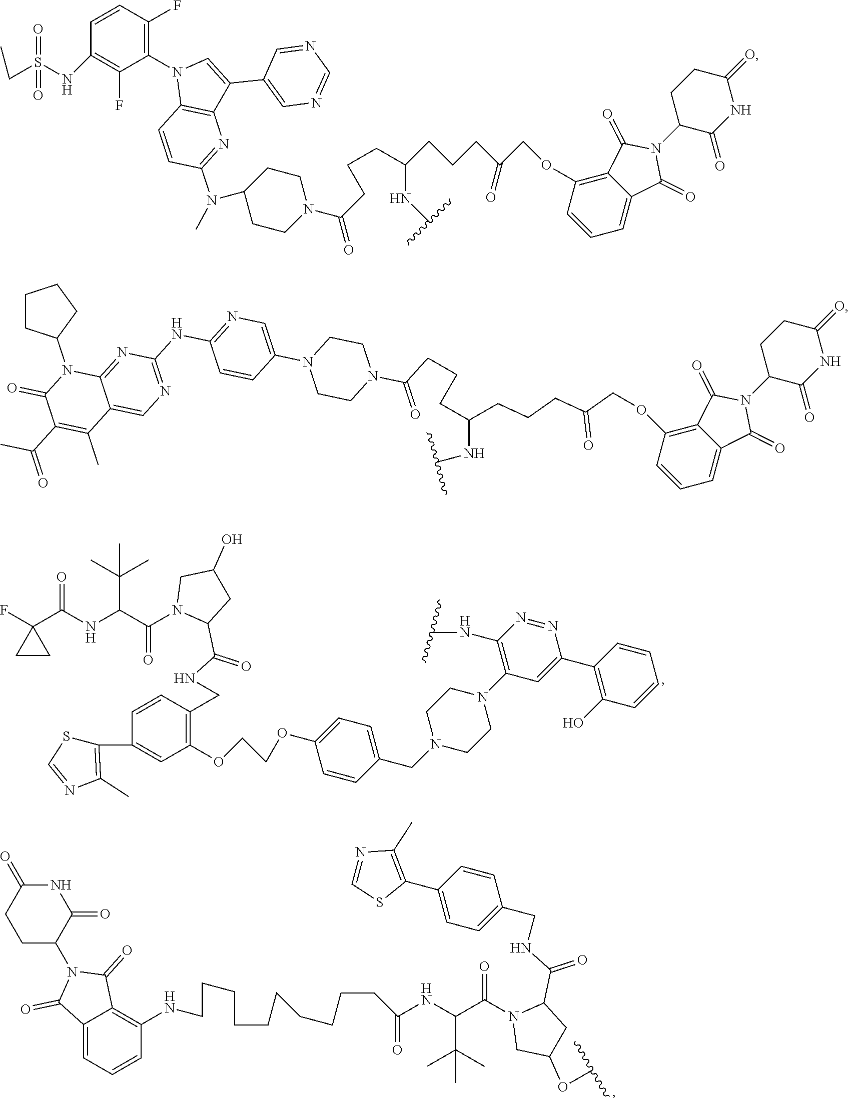 [SMILES: *NC(CCCC(=O)COc1cccc2c1C(=O)N(C1CCC(=O)NC1=O)C2=O)CCCC(=O)N1CCC(N(C)c2ccc3c(n2)c(-c2cncnc2)cn3-c2c(F)ccc(NS(=O)(=O)CC)c2F)CC1 ; *NC(CCCC(=O)COc1cccc2c1C(=O)N(C1CCC(=O)NC1=O)C2=O)CCCC(=O)N1CCN(c2ccc(Nc3ncc4c(C)c(C(C)=O)c(=O)n(C5CCCC5)c4n3)nc2)CC1 ; *Nc1nnc(-c2ccccc2O)cc1N1CCN(Cc2ccc(OCCOc3cc(-c4scnc4C)ccc3CNC(=O)C3CC(O)CN3C(=O)C(NC(=O)C3(F)CC3)C(C)(C)C)cc2)CC1 ; *OC1CC(C(=O)NCc2ccc(-c3scnc3C)cc2)N(C(=O)C(NC(=O)CCCCCCCCCCNc2cccc3c2C(=O)N(C2CCC(=O)NC2=O)C3=O)C(C)(C)C)C1]

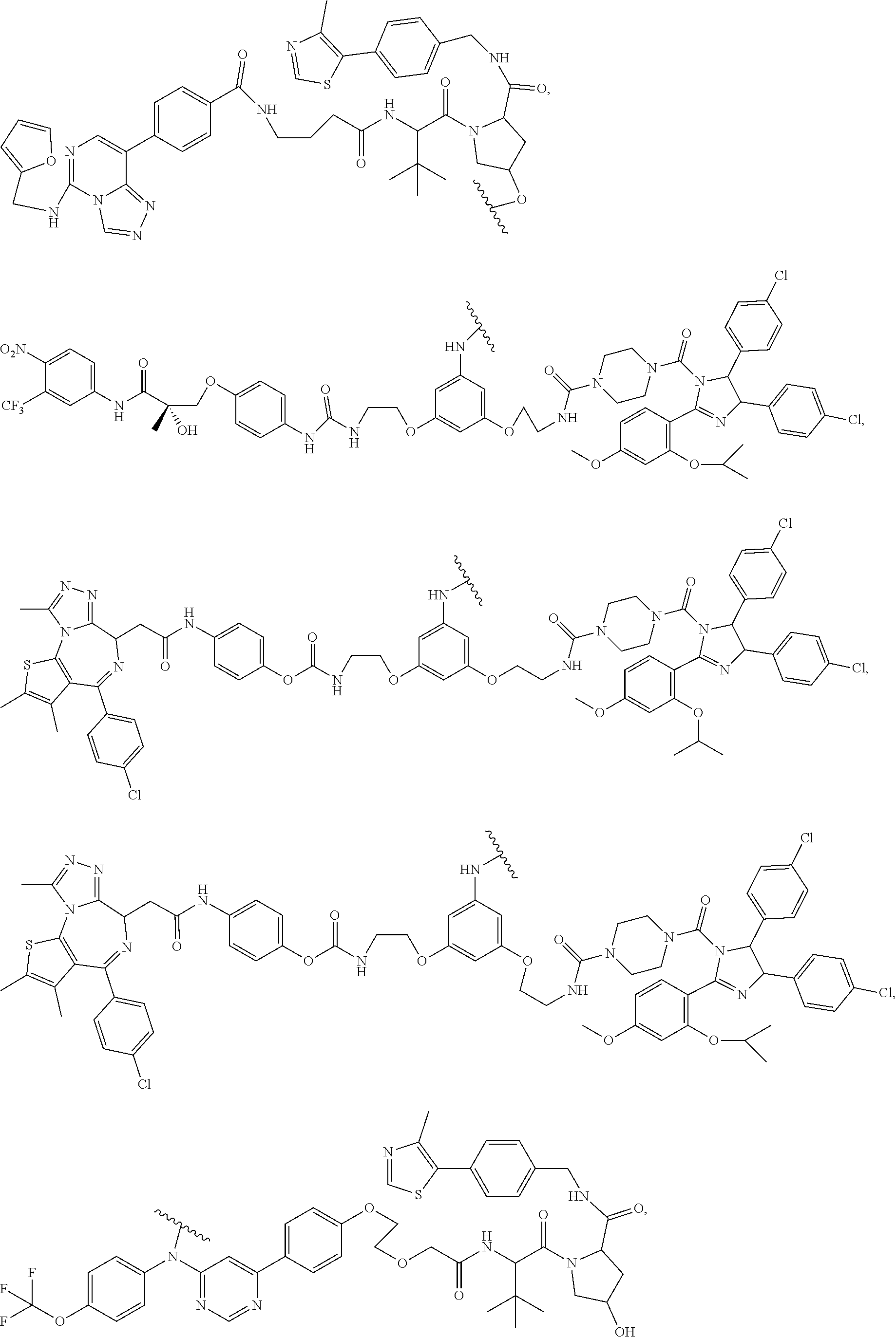

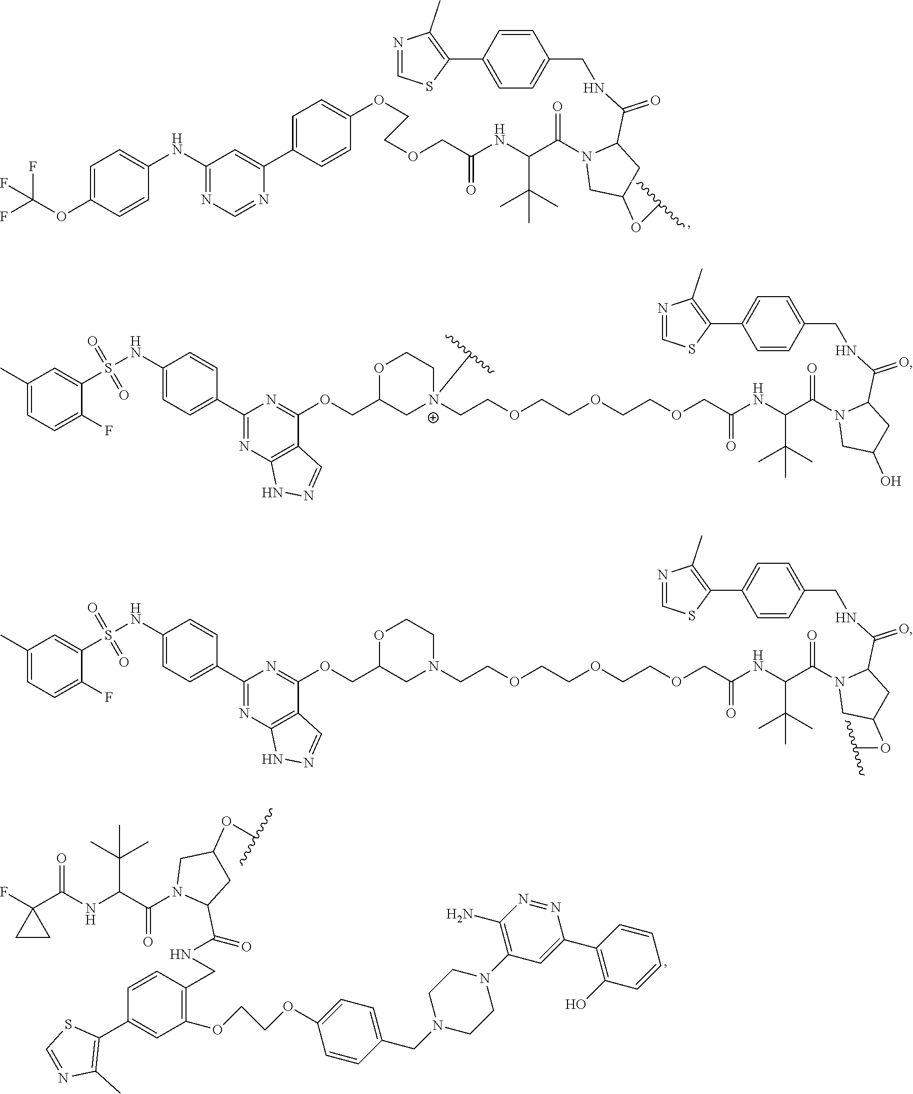

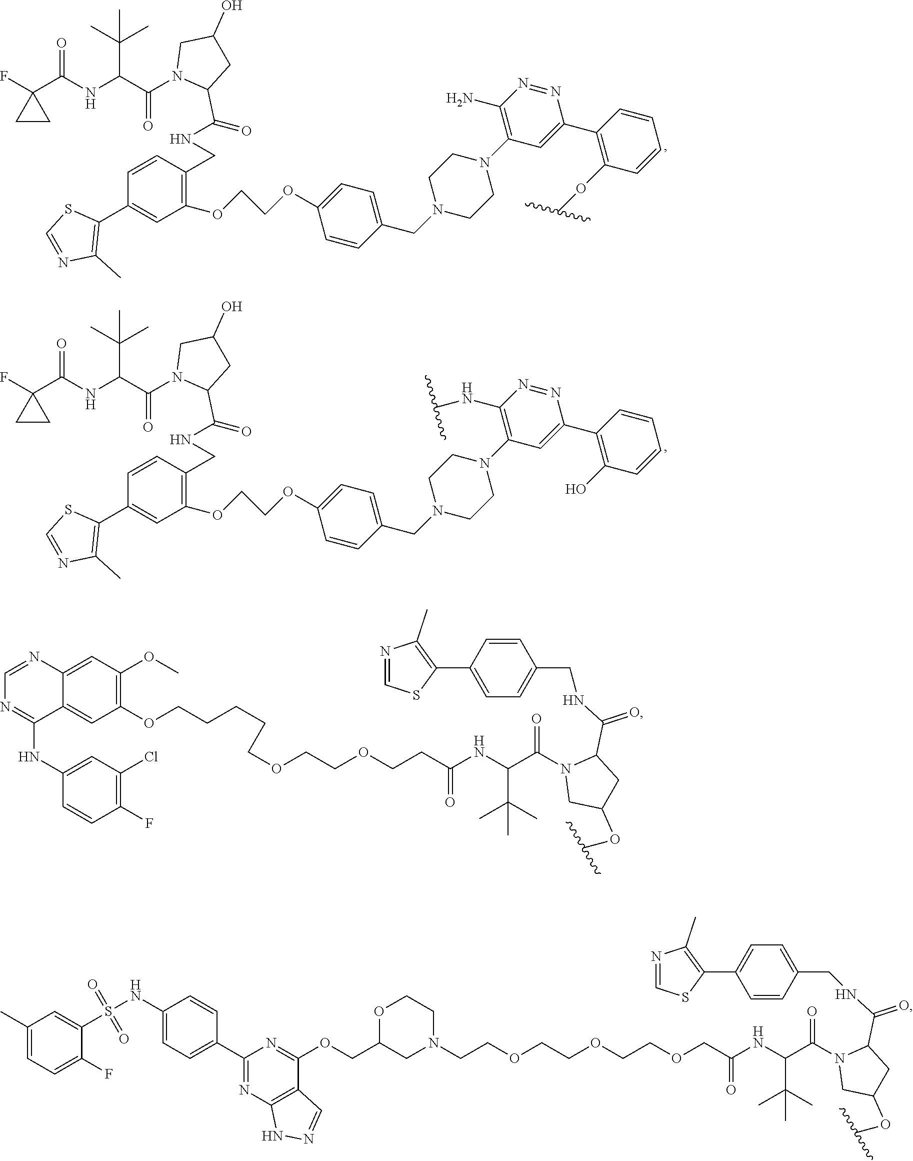

or hydrated salts; or the polymorphic crystalline structures of these compounds; or the optical isomers, racemates, diastereomers or enantiomers; wherein [symbol] is the linkage site;
In the further embodiments, a certain cell receptor agonist, a cell stimulating molecule or intracellular signaling molecule can be as a cytotoxic agent (drug 1 or/and drug 2) or the functional small molecule, A₁, A₂, A₃, A₄, A₅ and/or A₆ in Formula (I), (II), (III) or (IV), which is conjugated to the antibody of the invention. A cell-binding ligand or receptor agonist selected from: Folate derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (a-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), the dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones, selected from the group consisting of luteinizing hormone-releasing hormone (LHRH) agonists and antagonists, and gonadotropin-releasing hormone (GnRH) agonist, acts by targeting follicle stimulating hormone (FSH) and luteinizing hormone (LH), as well as testosterone production, selected from the group consisting of buserelin, Gonadorelin, Goserelin, Histrelin, leuprolide, Nafarelin, Triptorelin, Nafarelin, Deslorelin, Abarelix, Cetrorelix, Degarelix, and Ganirelix; Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of αvβ₁, αvβ₃, αvβ₅, αvβ₆, α₆β₄, α₇β₁, α_{L}β₂, α_{IIb}β₃) agonists (selected from the group consisting of GRGDSPK, cyclo(RGDfV) (L₁) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Anticalin (a derivative of Lipocalins); Adnectins (10th FN3 (Fibronectin)); Designed Ankyrin Repeat Proteins (DARPins); Avimers; EGF receptors, or VEGF receptors' agonists;

In the further embodiments, the functional small molecule, A₁, A₂, A₃, A₄, A₅ and/or A₆ in Formula (I), (II), (III) or (IV), is selected from a cell-binding molecule/ligand or a cell receptor agonist of the following molecules: LB01 (Folate), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αvβ3 integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR2 for a Toll-like receptor), LB23 (for an androgen receptor), LB24 (Cilengitide/cyclo (-RGDfV-) for an av integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) which are shown in the following structures:

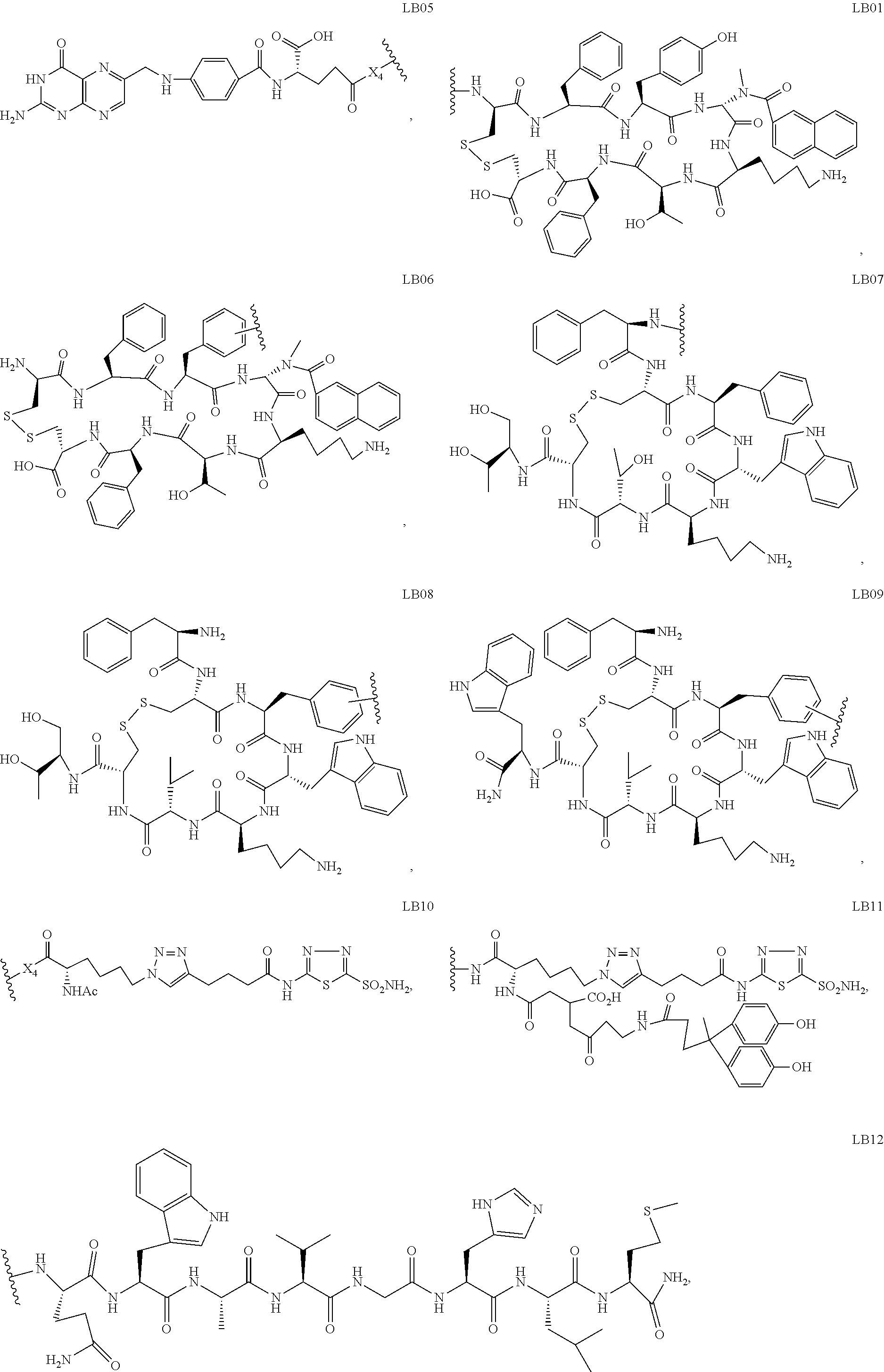

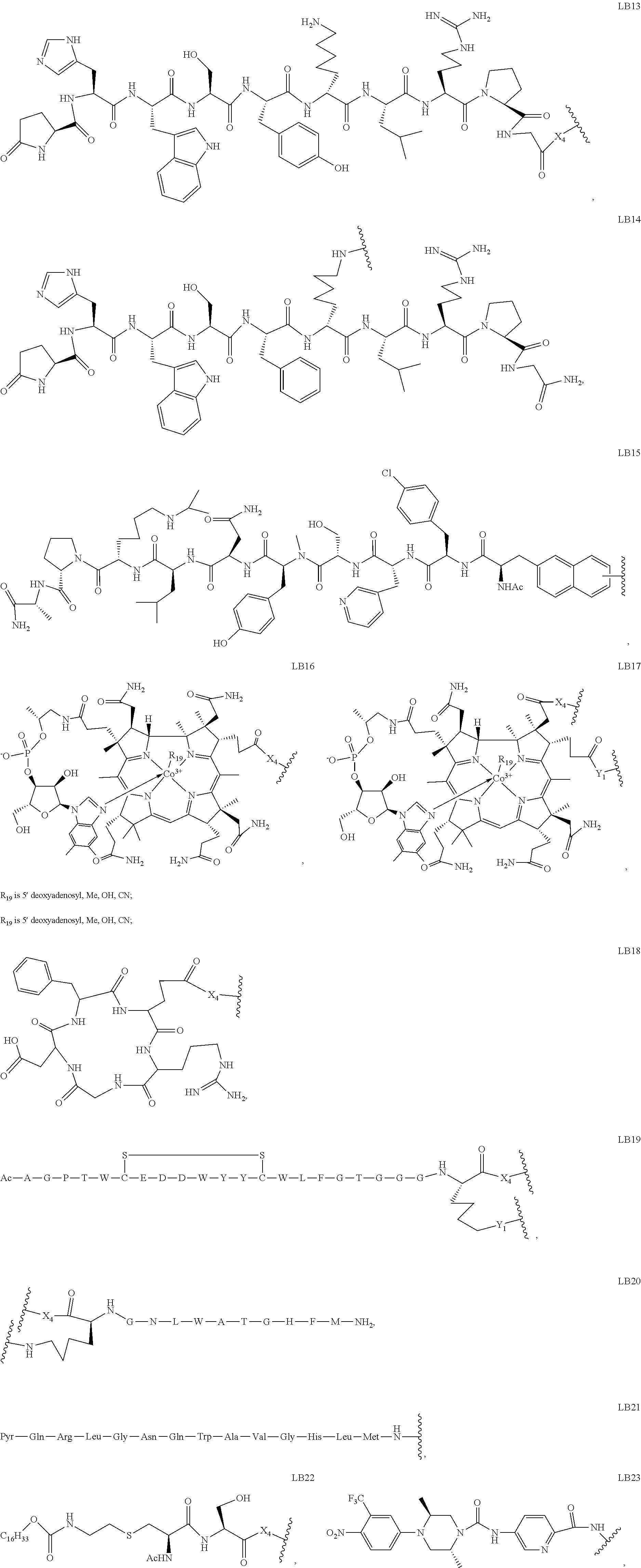

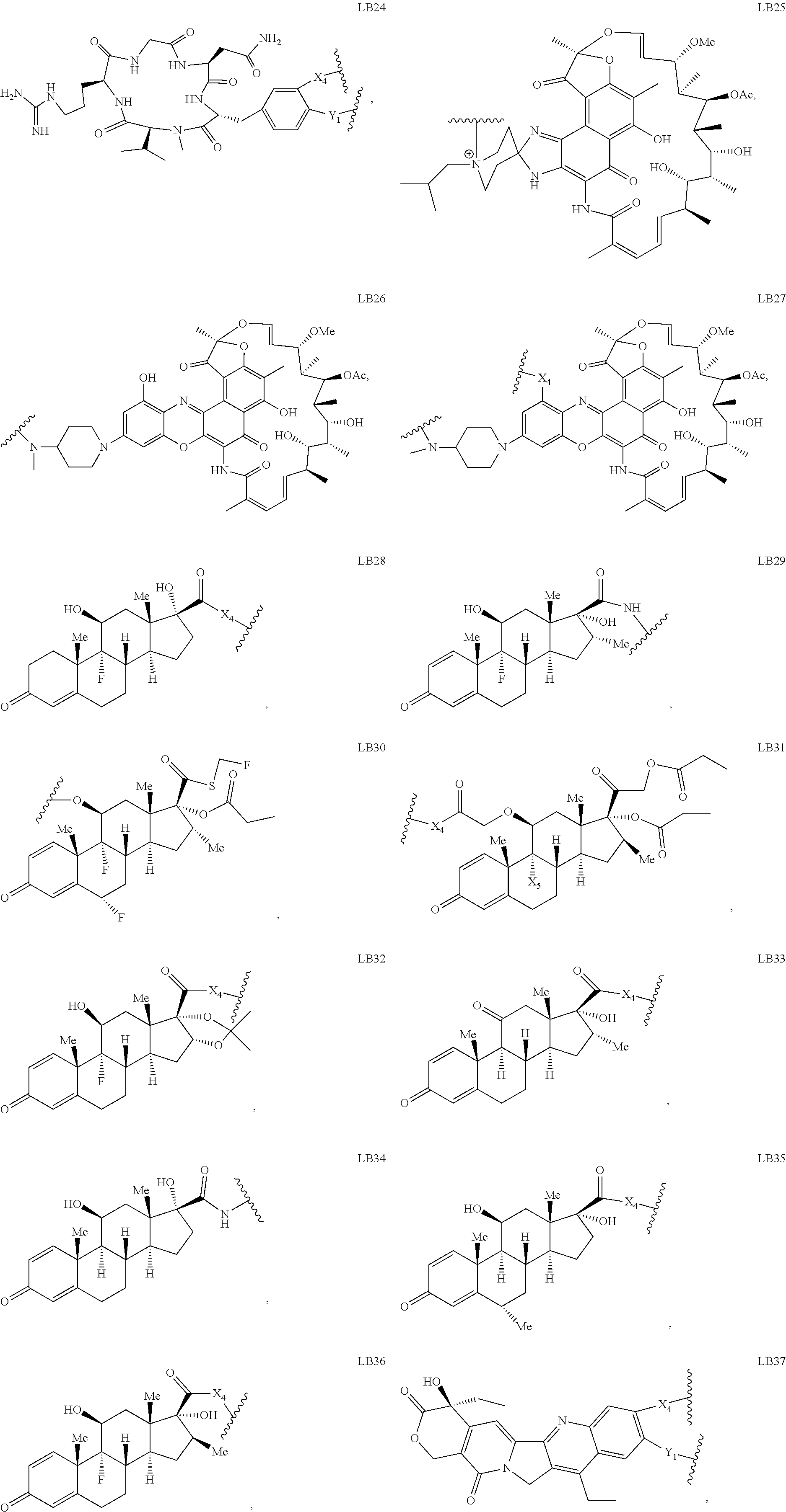

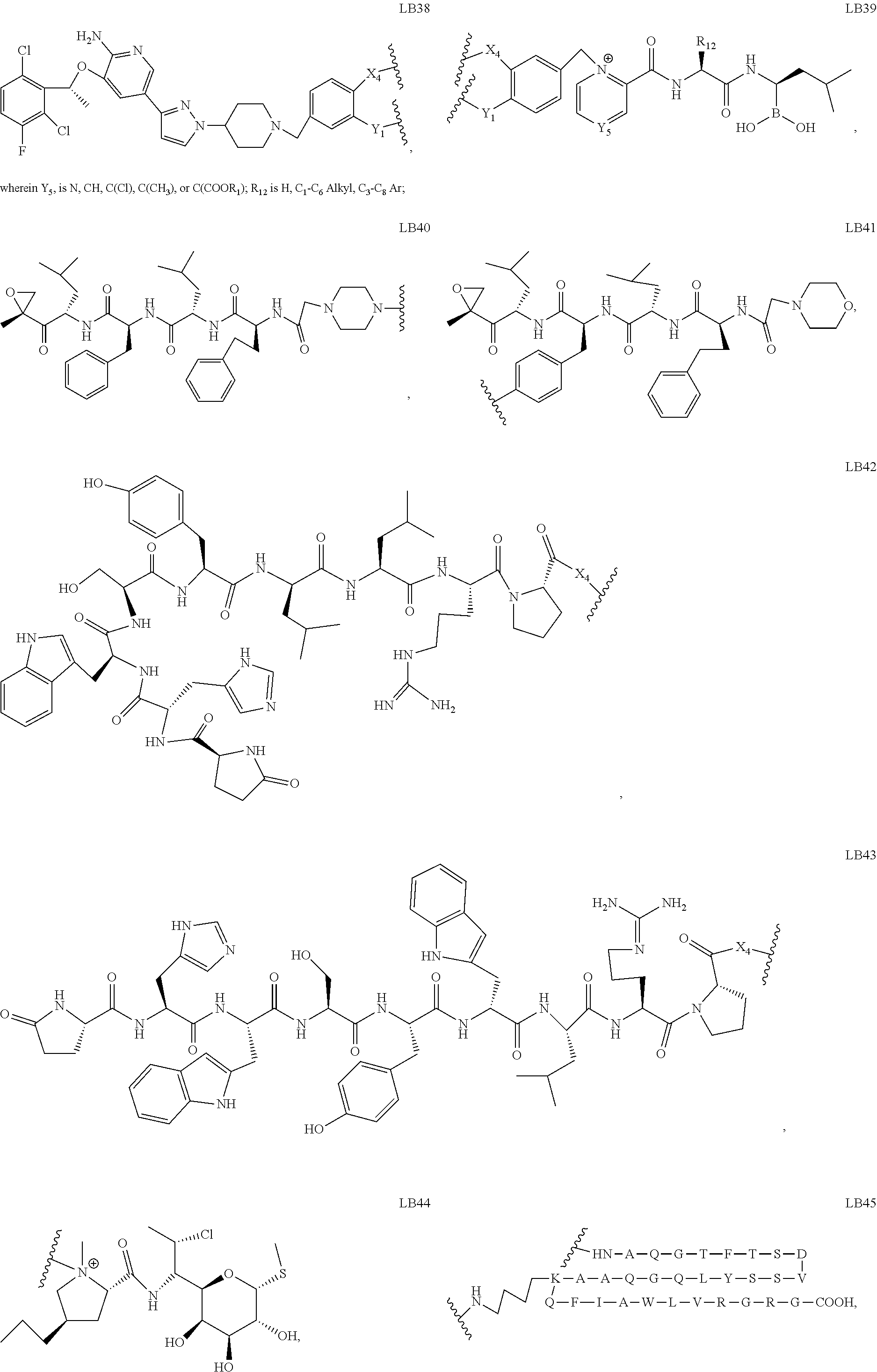

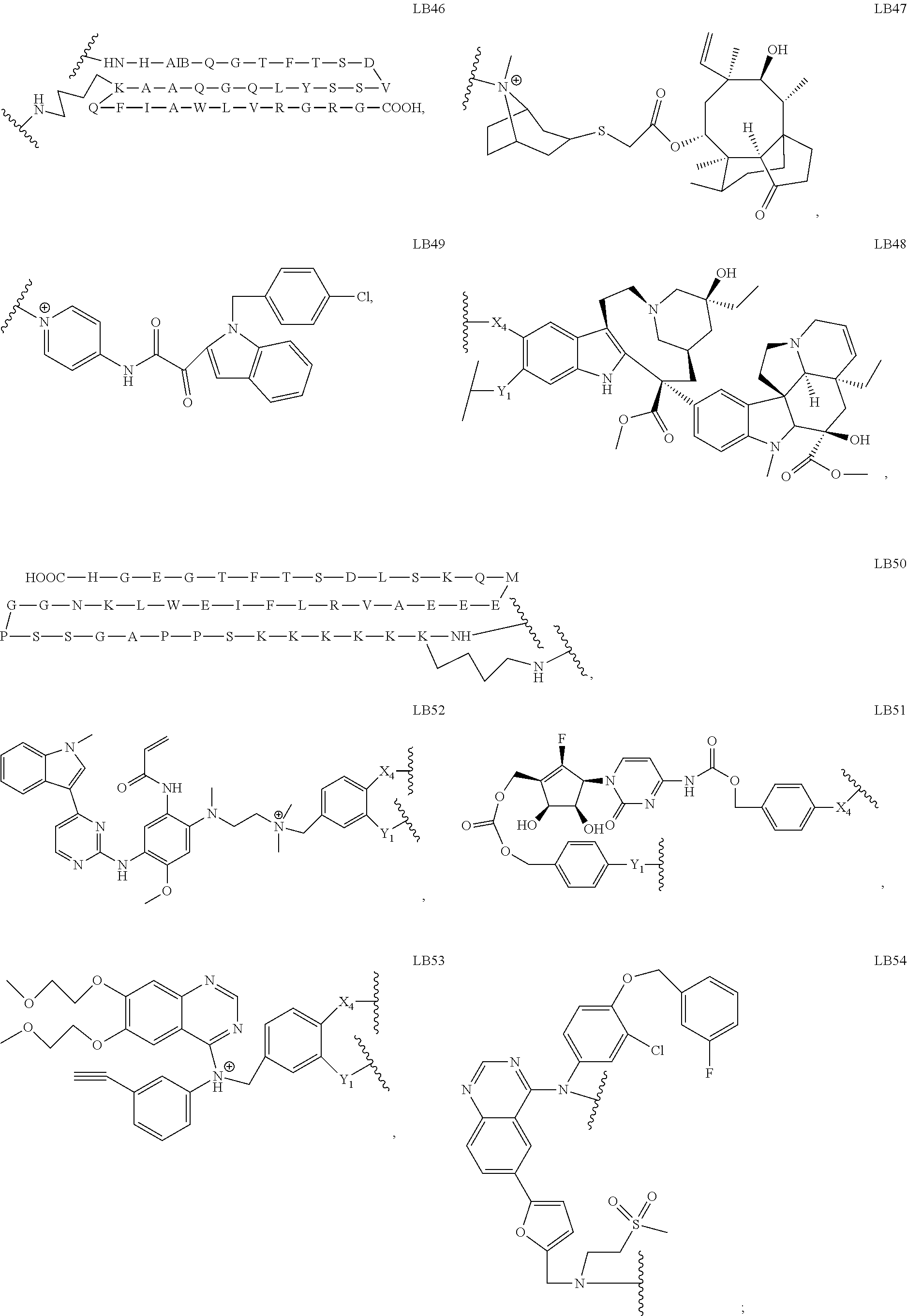

or hydrated salts; or the polymorphic crystalline structures of these compounds; or the optical isomers, racemates, diastereomers or enantiomers; wherein [symbol] is the linkage site; wherein X₄, and Y₁ are independently O, NH, NHNH, NR₁, S, C(O)O, C(O)NH, OC(O)NH, OC(O)O, NHC(O)NH, NHC(O) S, OC(O)N(R₁), N(R₁) C(O)N(R₁), CH₂, C(O)NHNHC(O) and C(O)NR₁, and R₁ is C₁-C₈ alkyl.

In another embodiments, the linker L₁, L₂, La₁, La₂, Lb₁, Lb₂, Lc₁ and Lc₂ are, the same or different, independently selected from O, NH, S, S—S, NHNH, N(R₃), N(R₃)N(R_{3′}), C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; C₂-C₈ (2-8 carbon atoms) of esters, ether, or amide; 1˜8 natural or unnatural amino acids described in the definition; polyethyleneoxy unit of formula (OCH₂CH₂)ₚ, (OCH₂CH(CH₃))ₚ, (OCH₂CH₂) POR₃, (OCH₂CH(CH₃)) POR₃, NH(CH₂CH₂O)ₚR₃, NH(CH₂CH(CH₃)O)ₚR₃,N[(CH₂CH₂—O)ₚR₃][(CH₂CH₂O)ₚR₃′], (OCH₂CH₂)ₚCOOR₃, or CH₂CH₂ (OCH₂CH₂)ₚCOOR₃, wherein p and p′ are independently an integer selected from 0 to about 1000, or combination thereof, wherein R₃ and R₃′ are independently H; C₁-C₈ of alkyl; C₂-C₈ of heteroalkyl, alkylcycloalkyl, heterocycloalkyl; C₃-C₈ of aryl, Ar-alkyl, heterocyclic, cycloalkyl, heteroalkylcycloalkyl, alkylcarbonyl, heteroaryl;
L₁, L₂, La₁, La₂, Lb₁, Lb₂, Lc₁ and Lc₂ may independently contain a self-immolative or a non-self-immolative component, peptidyl units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals.

Preferably, the self-immolative linker component has one of the following structures:

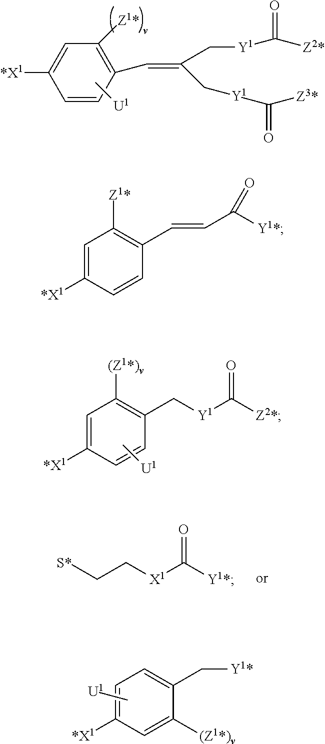

wherein the (*) atom is the point of attachment of additional spacer or releasable linker units, or the cytotoxic agent, and/or the antibody; X₁, Y₁, Z² and Z³ are independently NH, O, or S; Z¹ is independently H, NH, O or S; v is 0 or 1; U1 is independently H, OH, C₁˜C₆ alkyl, (OCH₂CH₂)ₙF, C₁, Br, I, OR₅, SR⁵, NR₅R₅′, N═NR₅, N═R₅, NR₅R₅′, NO₂, SOR₅R₅′, SO₂R₅, SO₃R₅, OSO₂R₅, PR⁵R₅′, POR₅R₅′, PO₂R₅R₅′, OPO(OR₅)(OR₅′), or OCH₂PO(OR₅ (OR₅′) wherein R₅ and R₅′ are as defined above; preferably R₅ and R₅′ are independently selected from H, C₁˜C₈ alkyl; C₂˜C₈ alkenyl, alkynyl, heteroalkyl; C₃˜C₈ aryl, heterocyclic, carbocyclic, cycloalkyl, heterocycloalkyl, heteroaralkyl, alkylcarbonyl; or pharmaceutical cation salts.

The non-self-immolative linker component is one of the following structures:

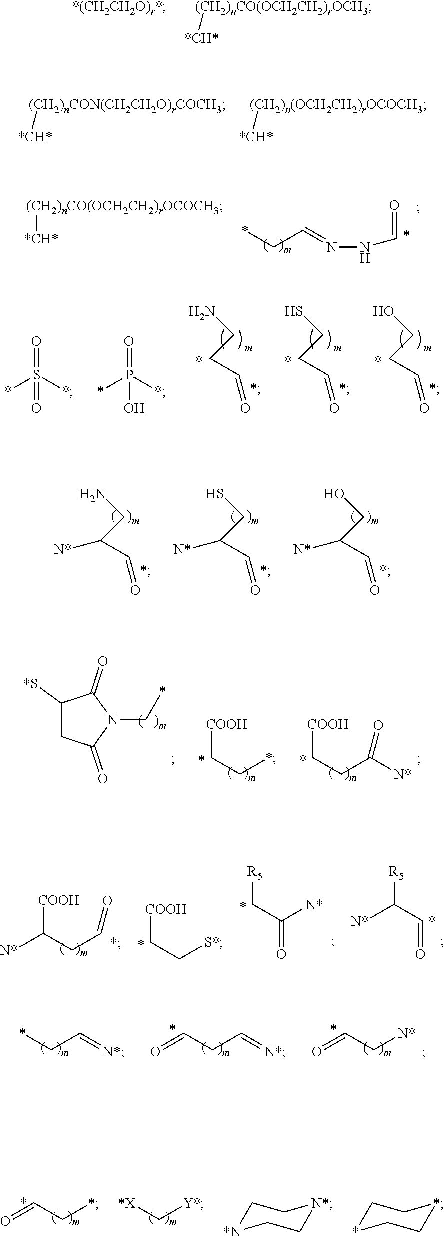

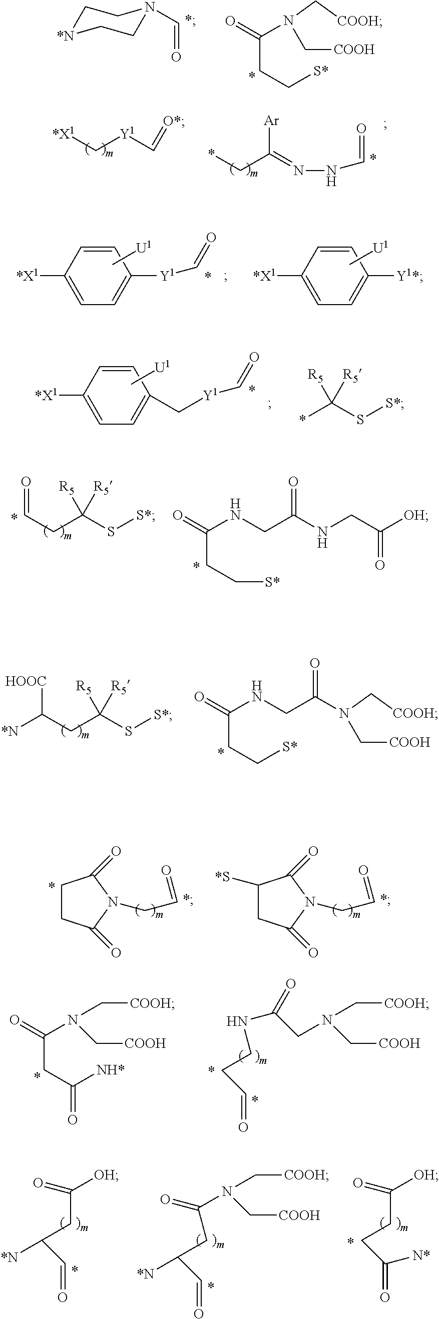

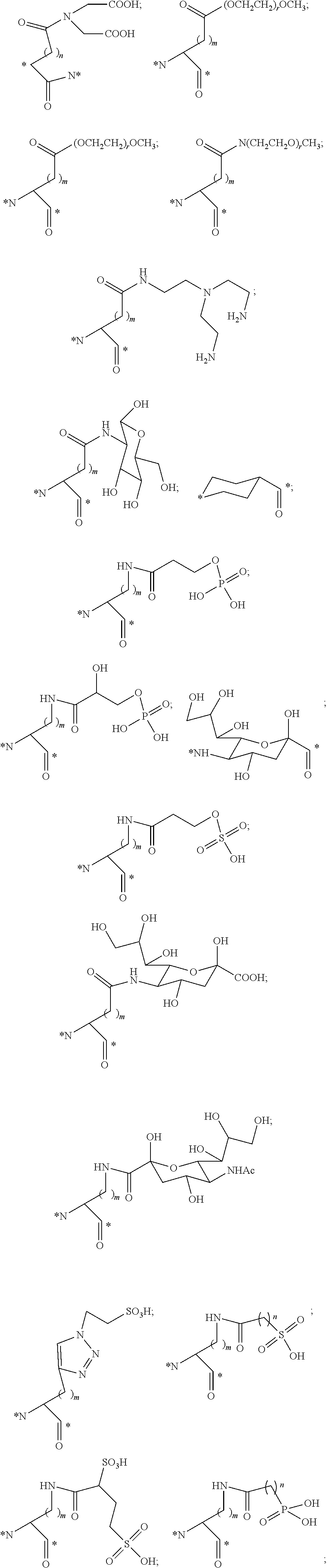

Wherein the (*) atom is the point of attachment of additional spacer R₁ or releasable linkers, the cytotoxic agents, and/or the binding molecules; X₁, Y₁, U1, R₁, R₅, R₅′ are defined as above; r is 0˜100; m and n are 0˜6 independently.

More preferably, L₁, L₂, La₁, La₂, Lb₁, Lb₂, Lc₁ and Lc₂ may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1˜8 natural or unnatural amino acid unites, wherein lysine, glutamic acid, aspartic acid, cysteine, or tyrosine may contain a side chain polyethyleneoxy unit of formula (OCH₂CH₂) POR₃, (OCH₂CH(CH₃)) POR₃, NH(CH₂CH₂O)ₚR₃, C(O)(CH₂CH₂O)ₚR₃, C(O)(CH₂CH₂OCH₂CH)ₚR₃, NH(CH₂CH(CH₃)O)ₚR₃, C(O)(CH₂OCH₂CH)ₚR₃, N[(CH₂CH₂O)ₚR₃][(CH₂CH₂O)ₚ′R₃′], (OCH₂CH₂)ₚCOOR₃, or CH₂CH₂ (OCH₂CH₂)ₚCOOR₃, NH(CH₂CH₂O)ₚCH₂CH₂R₃, wherein p and p′ are independently an integer selected from 0 to about 100, or combination thereof; wherein R₃ and R_{3′} are independently H, OH, NH₂, N(CH₂)₂, N(C₂H₅)₂, N(C₃H₇), C(═O) H, C(═O) CH₃, C₁-C₈ of alkyl, NH(C₄˜C₇ glycoside) or NH(C₄˜C₇ glycoside)₂; or combination above thereof;

Further preferably, L₁, L₂, La₁, La₂, Lb₁, Lb₂, Lc₁ and Lc₂ may independently be a releasable linker. The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells.

Examples of the releasable linkers (L, L₁ or L₂) include, but not limited:
—(CR₅R₆)ₘ(Aa)r(CR₇R₈)ₙ(OCH₂CH₂)ₜ—, —(CR₅R₆)ₘ(CR₇R₈)ₙ(Aa), (OCH₂CH₂)ₜ—, -(Aa), (CR₅R₆)ₘ(C R₇R₈)ₙ(OCH₂CH₂)ₜ—, —(CR₅R₆)ₘ(CR₇R₈)ₙ(OCH₂CH₂), (Aa)ₜ-, —(CR₅R₆)ₘ(CR₇—CR₈)—(CR₉R₁₀)ₙ(Aa)ₜ-(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ(Aa)ₜ-(NR₁₁CO)(CR₉R₁₀)ₙ—(OCH₂CH₂)ᵣ—, —(CR₅R₆)ₘ(OCO)(Aa)ₜ(CR₉R₁₀)ₙ—(OCH₂CH₂)ᵣ—, —(CR₅R₆)ₘ(OCNR₇)(Aa)ᵣ(CR₉R₁₀)ₙ—(OCH₂CH₂)ᵣ—, —(CR₅R₆)ₘ(CO)(Aa)ₜ-(CR₉R₁₀)ₙ(OCH₂CH₂)ᵣ—, —(CR₅R₆)ₘ(NR₁₁CO)(Aa)ₜ(CR₉R₁₀)ₙ—(OCH₂CH₂)ᵣ—, —(CR₅R₆)ₘ—(OCO)(Aa)ᵣ(CR₉R₁₀)ₙ—(OCH₂CH₂)ᵣ—, —(CR₅R₆)ₘ(OCNR₇)(Aa)ₜ(CR₉R₁₀)ₙ—(OCH₂CH₂)ᵣ—, —(CR₅R₆)ₘ(CO)(Aa)ₜ(CR₉R₁₀)ₙ—(OCH₂CH₂)ᵣ—, —(CR₅R₆)ₘ-phenyl-CO(Aa)ₜ(CR₇R₈)ₙ—, —(CR₅R₆)ₘ-furyl-CO(Aa)ₜ(CR₇R₈)ₙ—, —(CR₅R₆)ₘ-oxazolyl-CO(Aa)ₜ(CR₇R₈)ₙ—, —(CR₅R₆) mthiazolyl-CO-(Aa)ₜ(CCR7R₈)ₙ—, —(CR₅R₆)ₜ-thienyl-CO—(CR₇R₈)ₙ—, —(CR₅R₆)ₜ-imidazolyl-CO—(CR₇R₈)ₙ—, —(CR₅R₆)ₜ-morpholino-CO(Aa)ₜ-(CR₇R₈)ₙ—, —(CR₅R₆) tpiperazino-CO(Aa)ₜ(CR₇R₈)ₙ—, —(CR₅R₆)ₜ—N-methyl-piperazin-CO(Aa)ₜ-(CR₇R₈)ₙ—, —(CR₅R)ₘ-(Aa)ₜphenyl-, —(CR₅R₆)ₘ-(Aa)ₜfuryl-, —(CR₅R₆)ₘ-oxazolyl (Aa)ₜ-, —(CR₅R₆)ₘ-thiazolyl (Aa)ₜ-, —(CR₅R₆)ₘ-thienyl-(Aa)ₜ-, —(CR₅R₆)ₘ-imidazolyl(Aa)ₜ-, —(C R₅R₆)ₘ-morpholino-(Aa)ₜ-, —(CR₅R₆)ₘ-piperazino-(Aa)ₜ-, —(CR₅R₆)ₘN-methylpiperazino-(Aa)ₜ-,
—K(CR₅R₆)ₘ(Aa)ᵣ(CR₇R₈)ₙ(OCH₂CH₂)ₜ—, —K(CR₅R₆)ₘ(CR₇R₈)ₙ-(Aa)ᵣ(OCH₂CH₂)ₜ—, —K(Aa), (CR₅R₆)ₘ—(CR₇Rs)ₙ(OCH₂CH₂)ₜ—, —K(CR₅R₆)ₘ(CR₇R₈)ₙ—(OCH₂CH₂)ᵣ(Aa)ₜ-, —K(CR₅R₆)ₘ(CR₇—CR₈)(CR₉R₁₀)ₙ-(Aa)ₜ(OCH₂CH₂)ᵣ—, —K(CR₅R₆)ₘ—(NR₁₁CO)(Aa)ₜ(CR₉R₁₀)ₙ(OCH₂CH₂)ᵣ—, —K(CR₅R₆)ₘ(Aa)ₜ(NR₁₁CO)—(CR₉R₁₀)ₙ(OCH₂—CH₂)ᵣ—, —K(CR₅R₆)ₘ(OCO)(Aa)ₜ(CR₉R₁₀)ₙ—(OCH₂CH₂)ᵣ—, —K(CR₅R₆)ₘ(OCNR₇) (Aa)ₜ-(CR₉R₁₀)ₙ—(OCH₂CH₂)ᵣ—, —K(CR₅R₆)ₘ(CO)(Aa)ₜ-(CR₉R₁₀)ₙ(OCH₂CH₂)ᵣ—, —K(CR₅R₆)ₘ(NR₁₁CO)-(Aa)ₜ(CR₉R₁₀)ₙ(OCH₂CH₂)ᵣ—, —K(CR₅R₆)ₘ—(OCO)(Aa)ₜ(CR₉R₁₀)ₙ(OCH₂CH₂)ᵣ—, —K(CR₅R₆)ₘ(OCNR₇)-(Aa)ₜ(CR₉R₁₀)ₙ(OCH₂CH₂)ᵣ—, —K(CR₅R₆)ₘ(CO)(Aa)ₜ(CR₉R₁₀)ₙ—(OCH₂CH₂)ᵣ—, —K(CR₅R₆)ₘ- phenyl-CO-(Aa)ₜ(CR₇R₈)ₙ—, —K—(CR₅R₆)ₘ-furyl-CO(Aa)ₜ-(CR₇R₈)ₙ—, —K(CR₅R₆)ₘ-oxazolyl-CO(Aa)ₜ(CR₇R₈)ₙ—,
—K(CR₅R₆) mthiazolyl-CO(Aa)ₜ-(CR₇Rs)ₙ-, —K(CR₅R₆)ₜ-thienyl-CO(CR₇R₈)ₙ—, —K(CR₅R₆)ₜimidazolyl-CO—(CR₇R₈)ₙ—, —K(CR₅R₆)ₜmorpholino-CO(Aa)ₜ(CR₇Rs)ₙ-, —K(CR₅R₆) (piperazino-CO(Aa)ₜ-(CR₇Rs)ₙ-,
—K(CR₅R₆)ₜ—N-methylpiperazinCO(Aa)ₜ(CR₇R₈)ₙ—, —K(CR₅R)ₘ(Aa): phenyl, —K—(CR₅R₆)ₘ-(Aa)tfuryl-, —K(CR₅R₆)ₘ-oxazolyl(Aa)ₜ-, —K(CR₅R₆)ₘ-thiazolyl(Aa)ₜ-, —K(CR₅R₆)ₘ-thienyl-(Aa)ₜ-, —K(CR₅R₆)ₘ-imidazolyl(Aa)ₜ-, —K(CR₅R₆)ₘ-morpholino(Aa)ₜ-, —K(CR₅R₆)ₘpiperazino-(Aa)ₜG, —K(CR₅R₆)ₘN—
methylpiperazino-(Aa)ₜ-; wherein m, n, R₃, R₄, and R₅ are described above; Aa is an amino acid, t and r are 0-100 independently; R₆, R₇, and R₅ are independently chosen from H; halide; C₁˜C₈ of alkyl, aryl, alkenyl, alkynyl, ether, ester, amine or amide, which optionally substituted by one or more halide, CN, NR₁R₂, CF₃, OR₁, Aryl, heterocycle, S(O)R₁, SO₂R₁, —CO₂H, —SO₃H, —OR₁, —CO₂R₁, —CONR₁, —PO₂R₁R₂, —PO₃H or P(O)R₁R₂R₃; K is NR₁, —SS—, —C(═O)—, —C(═O)NH—, —C(═O)O—, —C═NH—O—, —C═N—NH—, —C(—O)NH—NH—, O, S, Se, B or C₃-C₆ heteroaromatic group.

Example structures of the components of the linker L₁, L₂, La₁, La₂, Lb₁, Lb₂, Lc₁ and Lc₂ may independently contain one or several of the following structures:

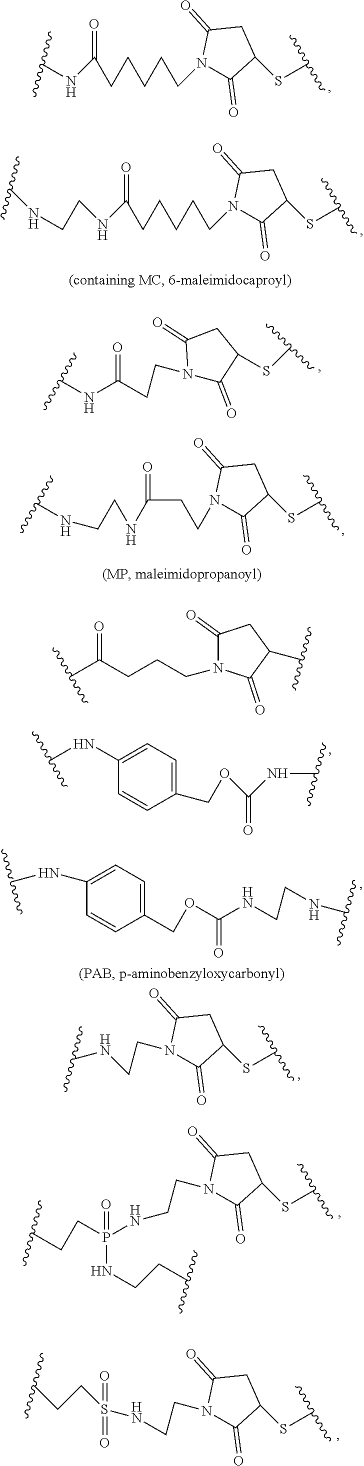

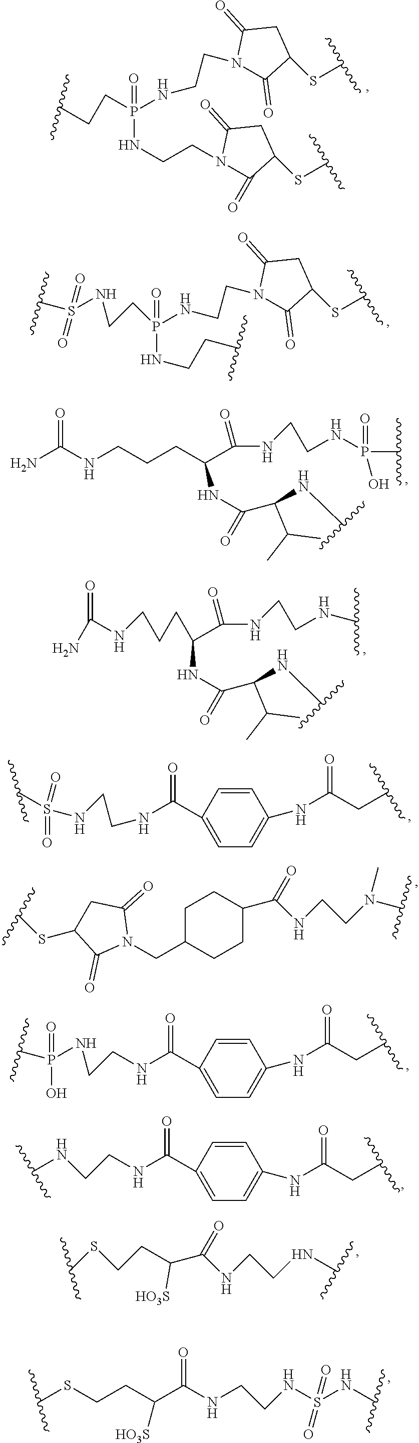

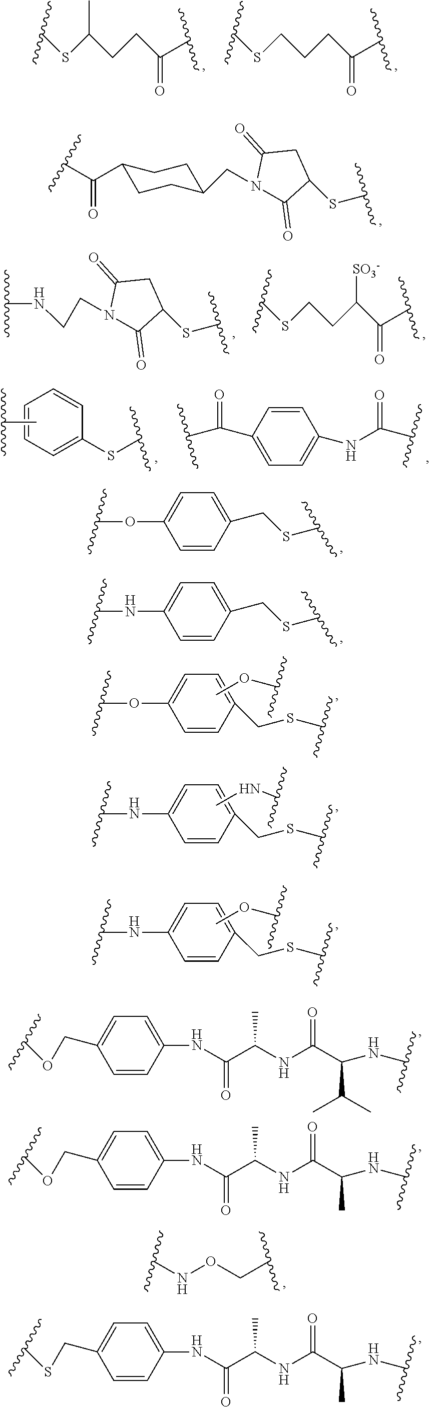

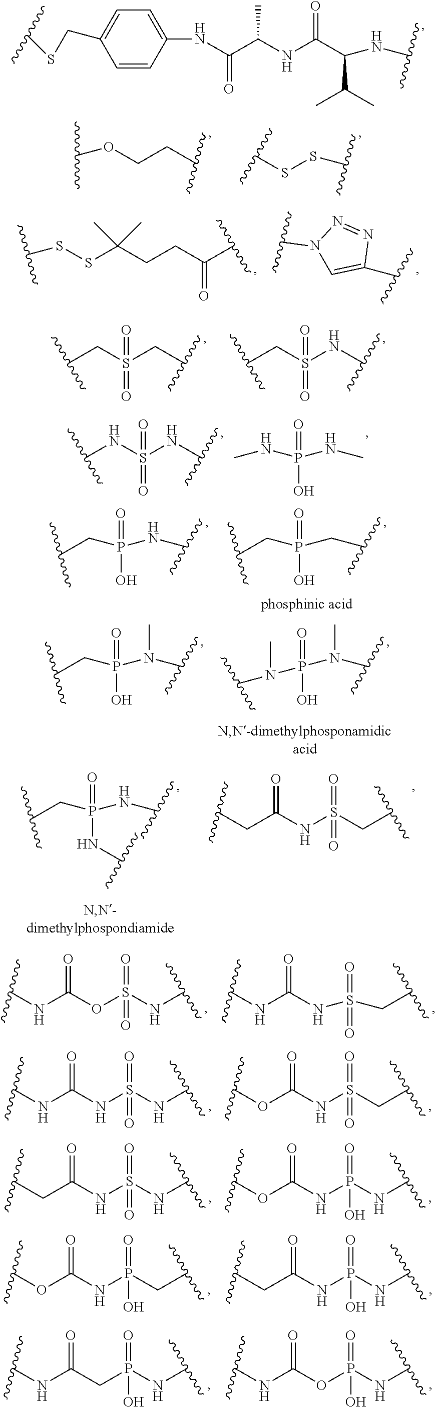

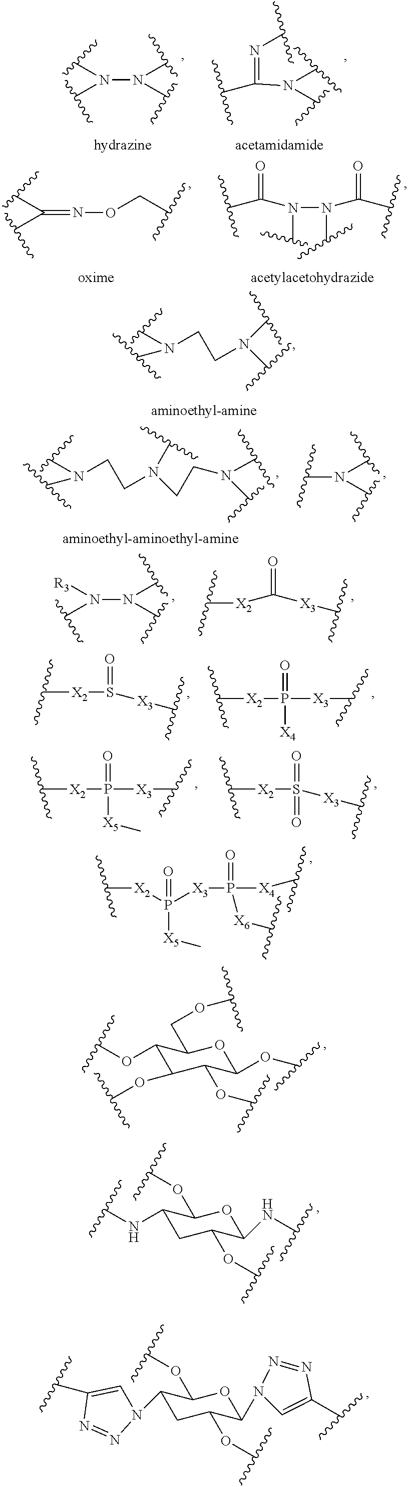

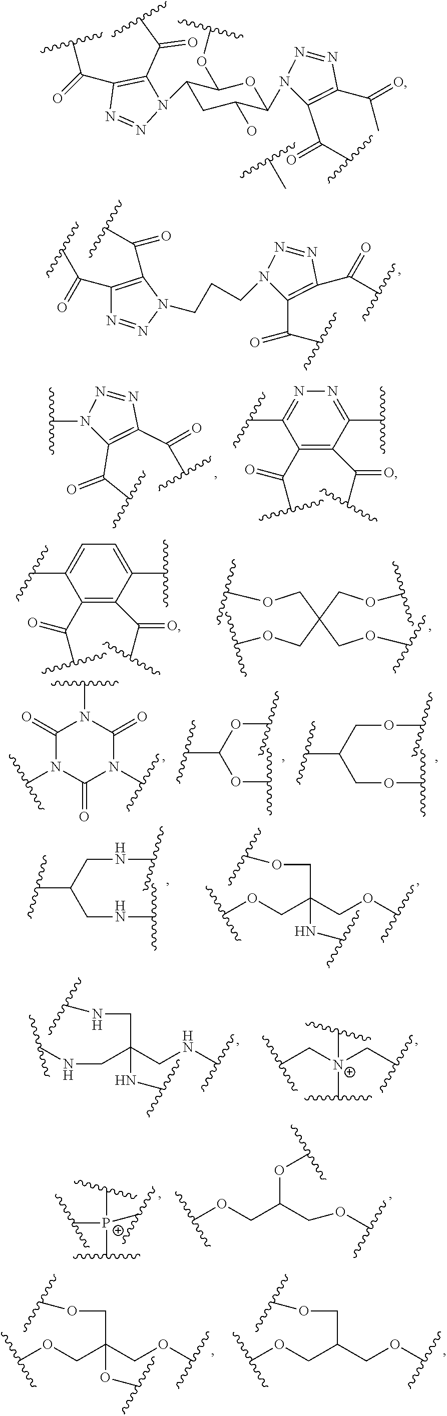

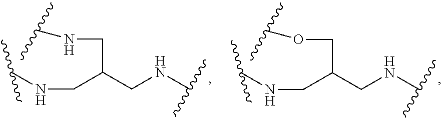 [SMILES: *NCC(CN*)CN* ; *NCC(CN*)CO*]

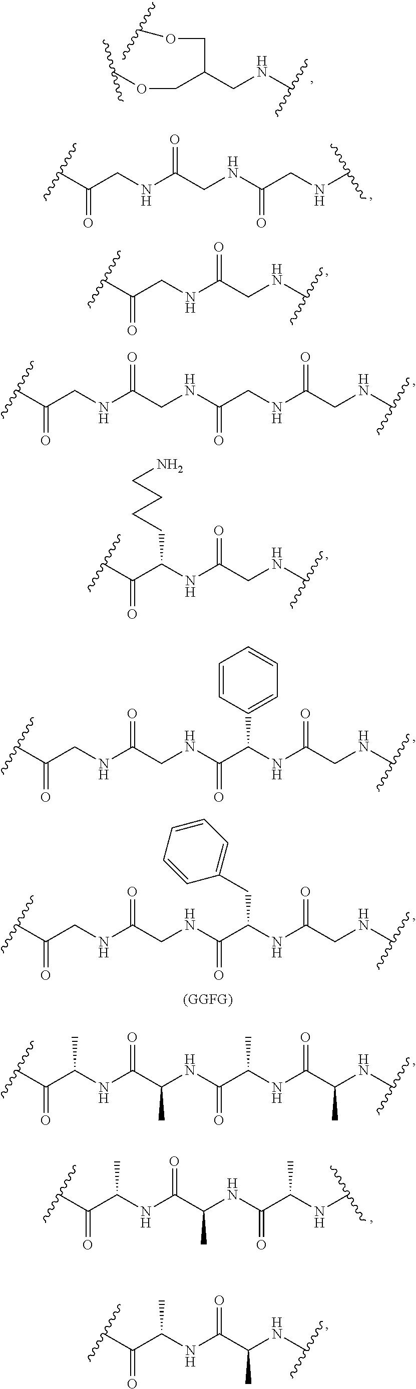

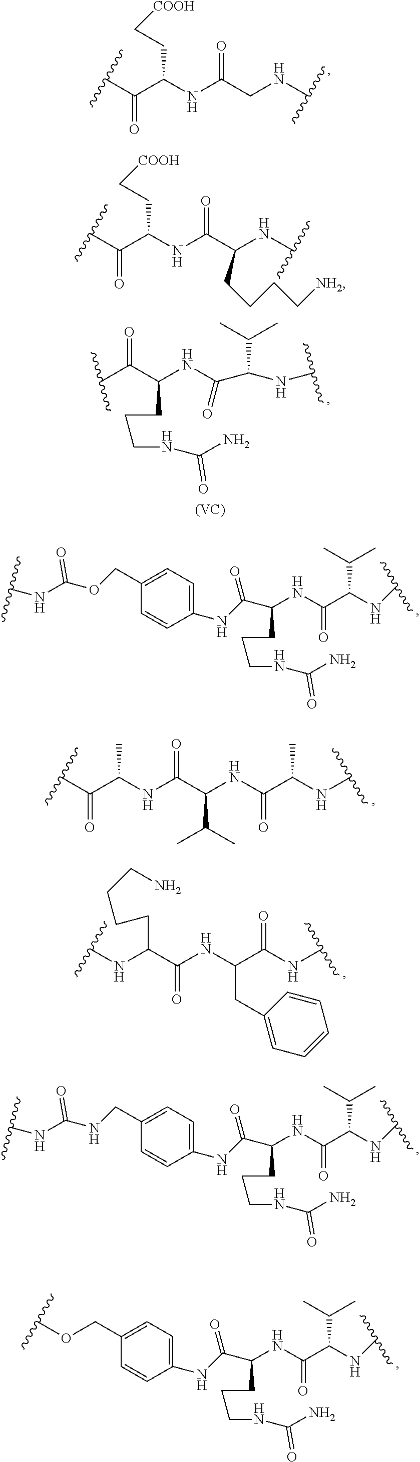

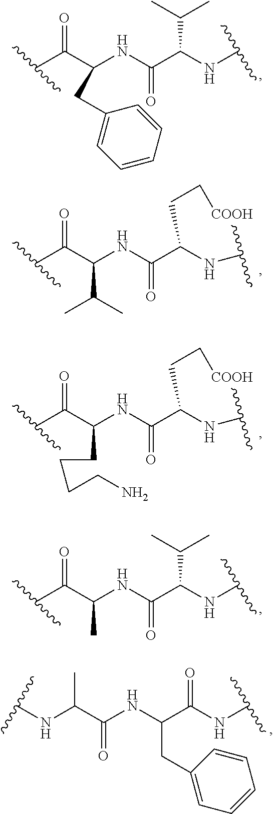

or a combination above thereof; wherein [symbol] is the site of linkage; X₂, X₃, X₄, X₅, or X₆, are independently selected from NH; NHNH; N(R₁₂); N(R₁₂) N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR¹², CH₂NHR₁₂, or 1˜8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

In another embodiments, the L₁, L₂, La₁, La₂, Lb₁, Lb₂, E₁, E₂, Lv₁′, Lv₂′, Lc₁ and Lc₂ that are jointly constructed in the structures of Formula (I), (II) and (III) are (IV) are accordingly selected from the following preferences:
wherein the structure of

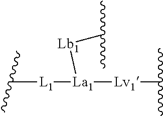

in formula (I) is preferably having the structure of formula (Ia); wherein the structure of

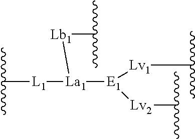

in formula (II) is preferably having the structure of formula (Ib) or (Ic); wherein the structure of

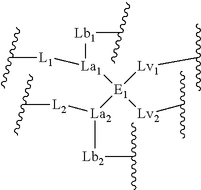

in formula (III) is preferably having the structure of formula (Id), (Ie), (If) or (Ig), or wherein

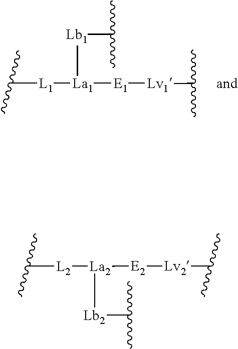

in formula (IV), is preferably having the structure of formula (Ia), illustrated as following:

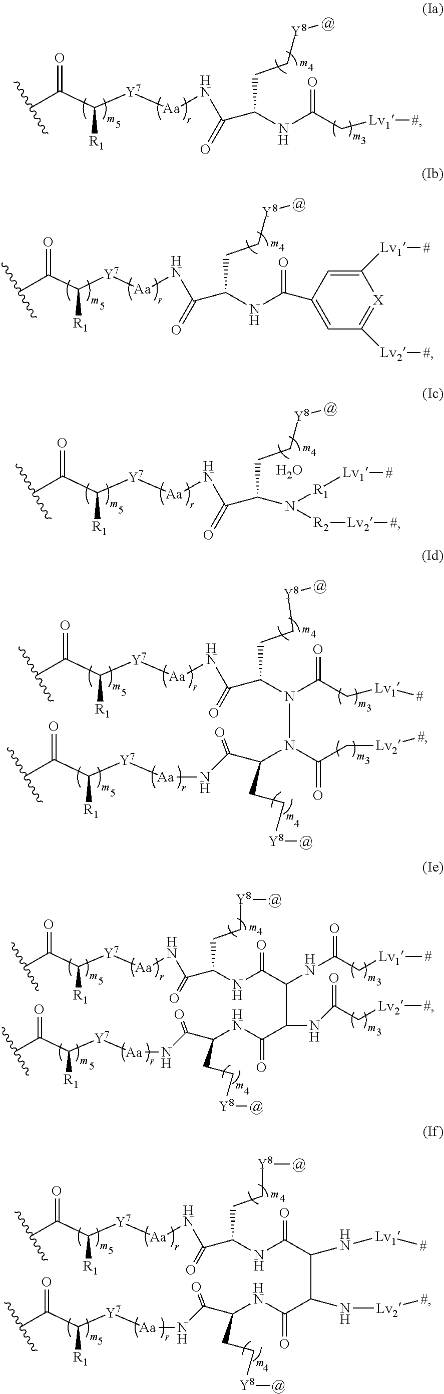

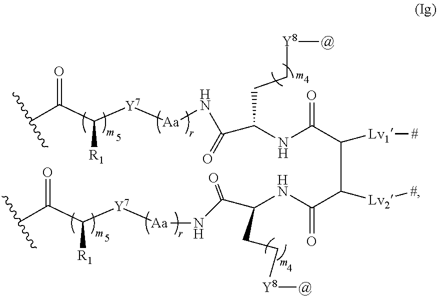

Wherein “ [symbol] ” is a site that links a drug or a site of linker L₁ or L₂; “#” is a site that links a S (thiol), O (phenol), NH (amino), CHO (aldehyde), C(═O) (ketone), C(O) (NH) (amide) and C(O)(OH) (carboxylate) of an antibody; Aa is L- or D-natural or unnatural amino acids; “@” is a site that links Lc₁ or Lc₂ described in the formula (I), (II), (III) and (IV);
R₁ is H, C₁-C₈alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0-8 (0, 1, 2, 3, 4, 5, 6, 7, or 8); when r is not 0, (Aa) r is the same or different amino acids or peptide units; when r=0, (Aa), is absent;
m₁=1-18 (0, 1,2,3,4,5,6, . . . , 18); m₂=1-100 (1, 2, 3, 4, 5, 6, 7,8, . . . , 100); m₃=1-8 (1, 2, 3, 4, 5, 6, 7, or 8); m₄=0-8 (0, 1, 2, 3, 4, 5, 6, 7, or 8); m₅=1-8 (1, 2, 3, 4, 5, 6, 7, or 8);
Y₇ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y₈ is NHC(═O), NHS(O₂), NH(SO), NHS(O₂)NH, NHP(O)(OH)NH, C(O)NH, OC(O)NH, NHC(O)NH, C(O), N, NH, CH₂, or CH;
Lv₁′ and Lv₂′ are independently or jointly selected from:

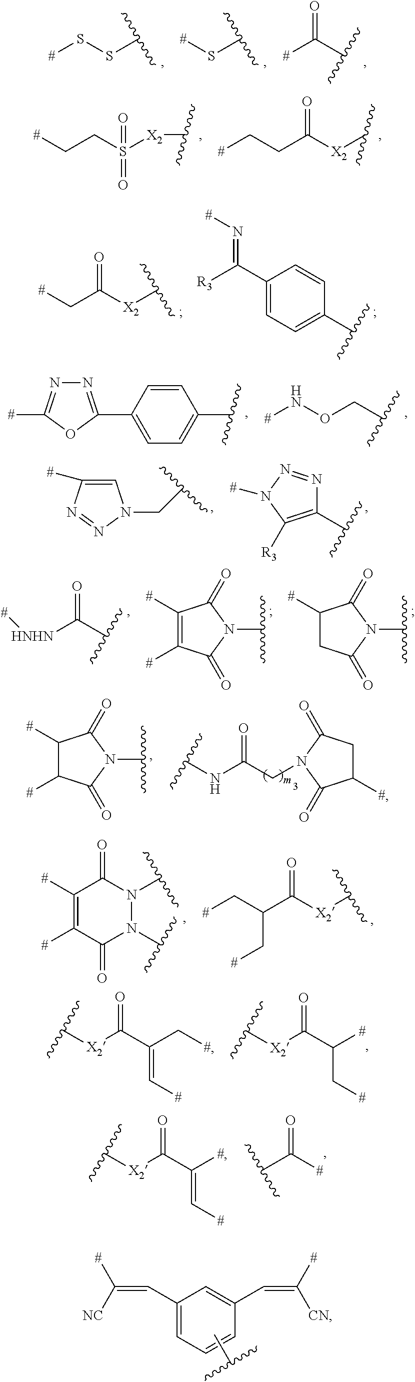

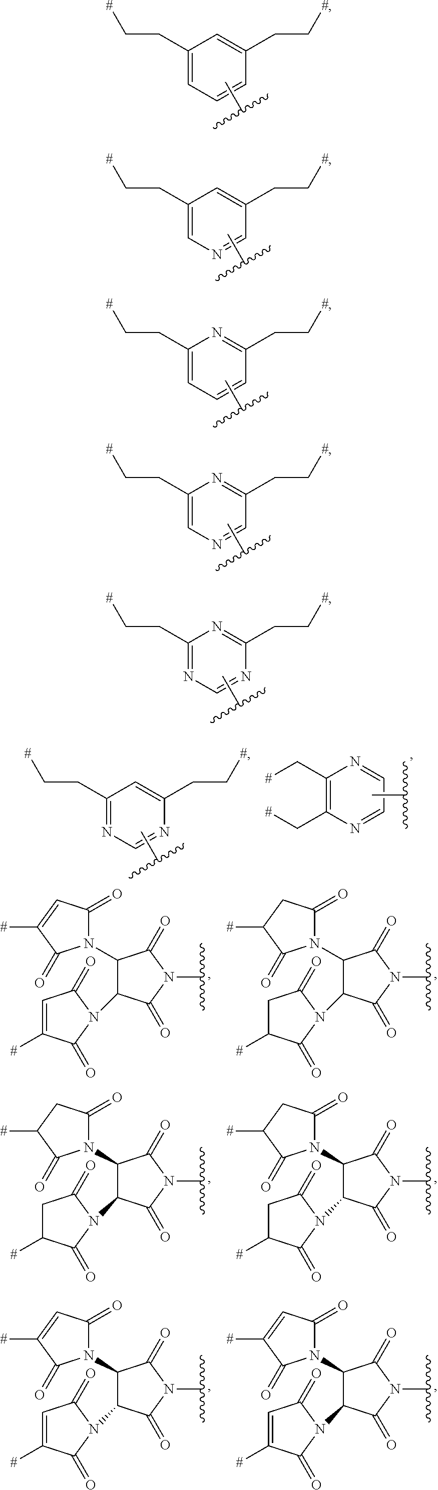

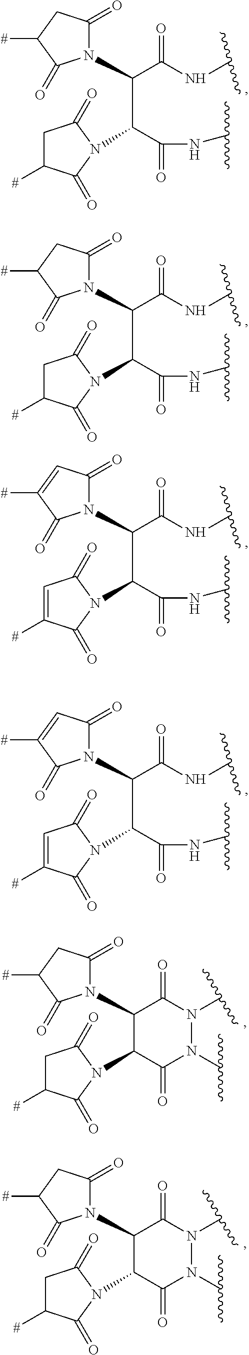

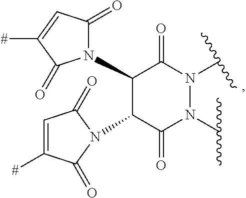

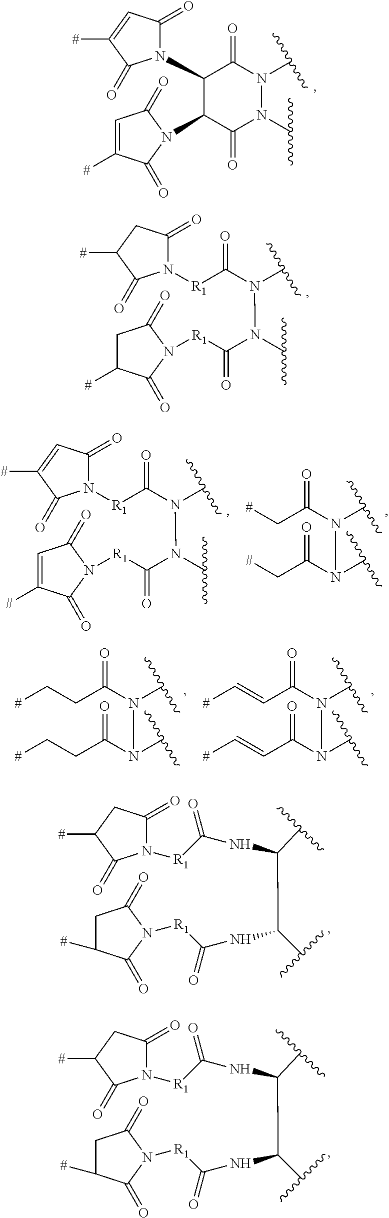

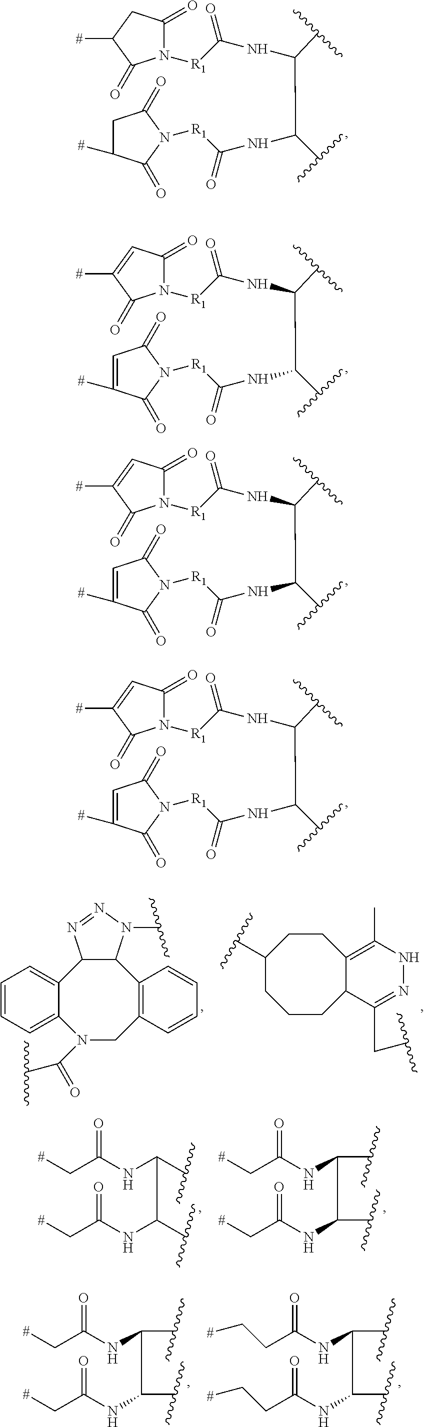

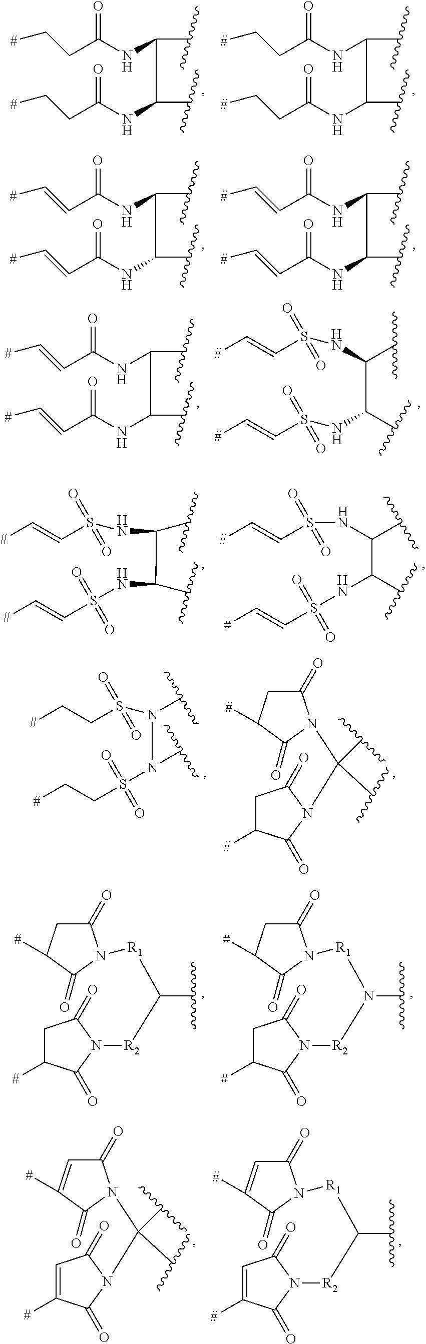

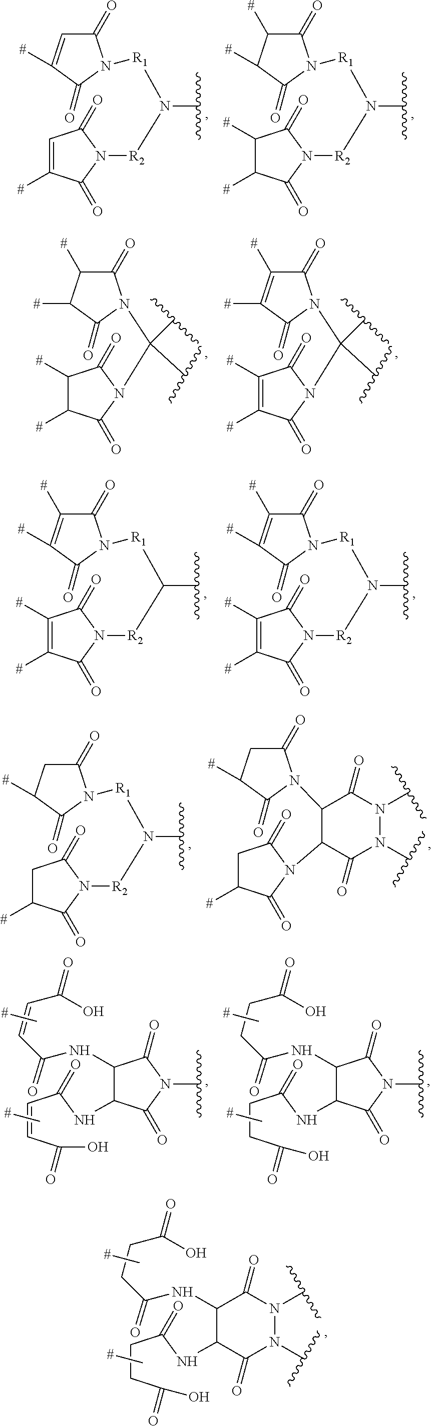

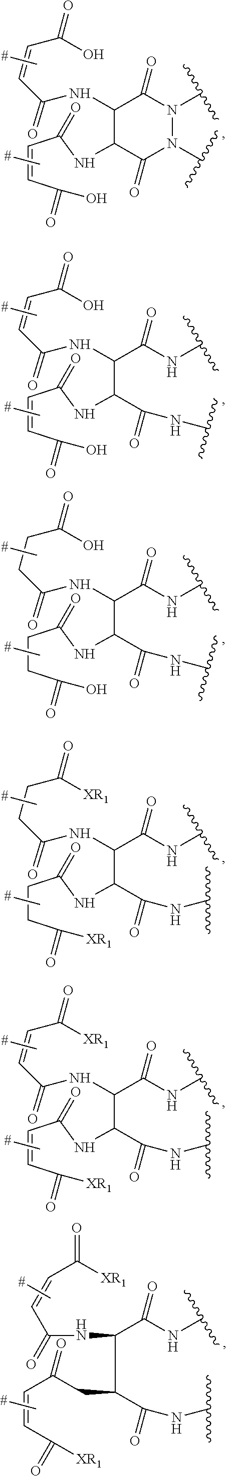

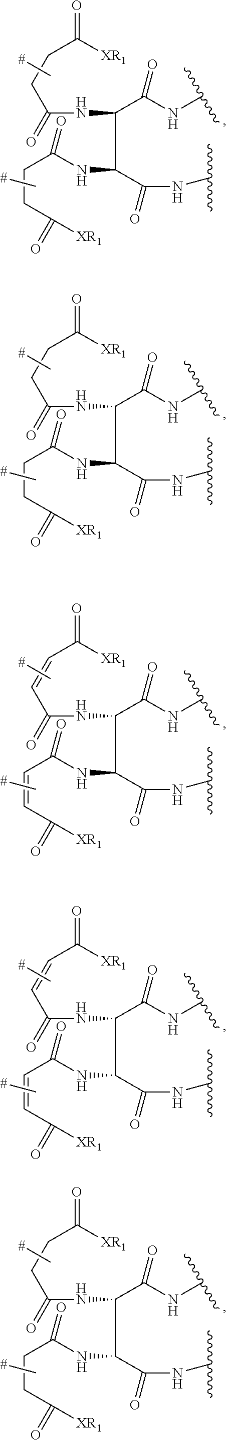

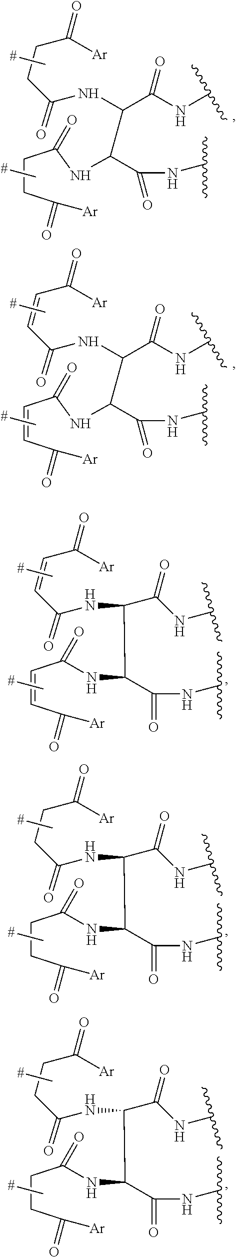

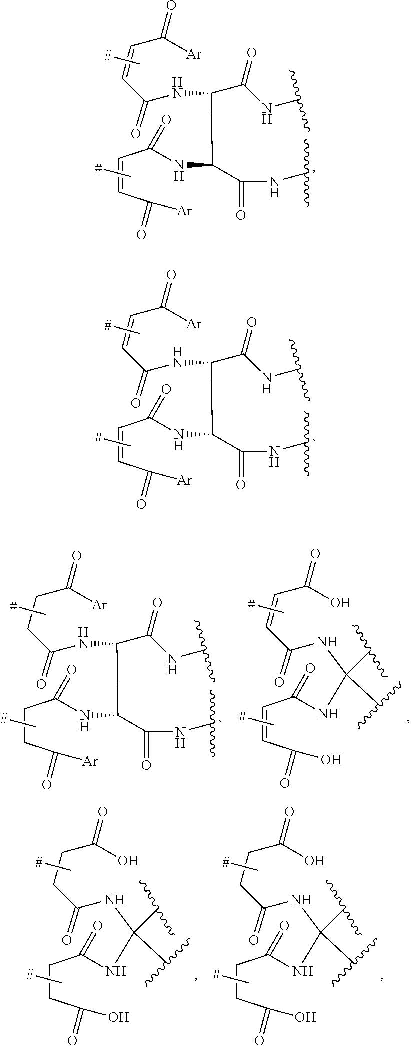

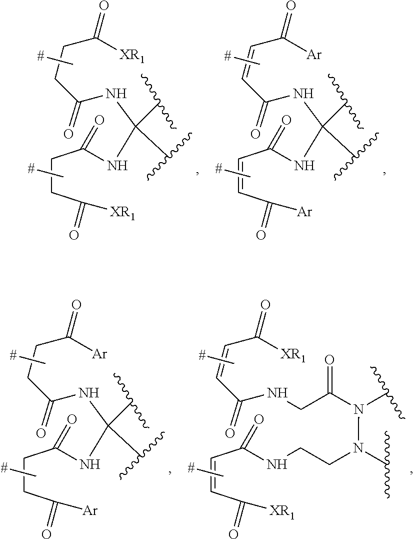

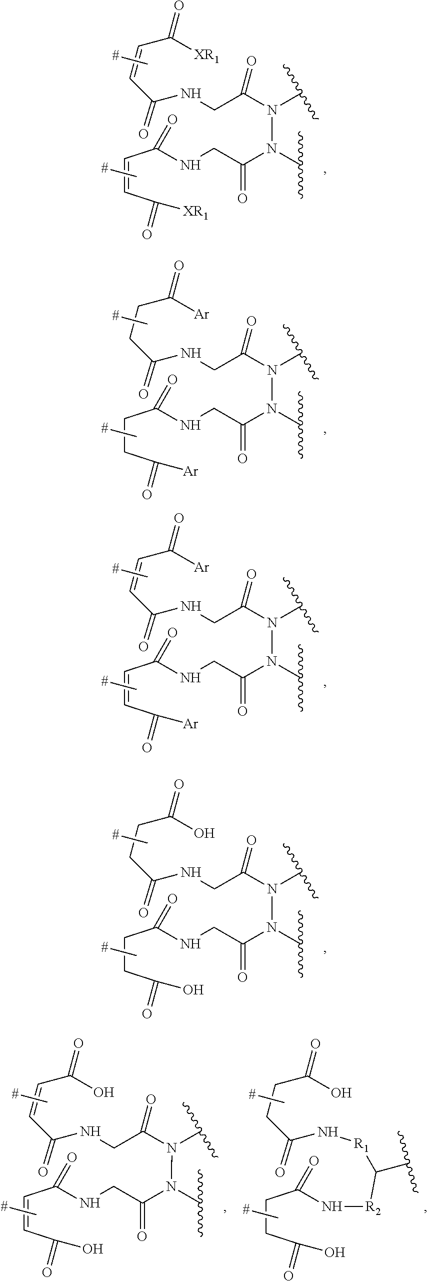

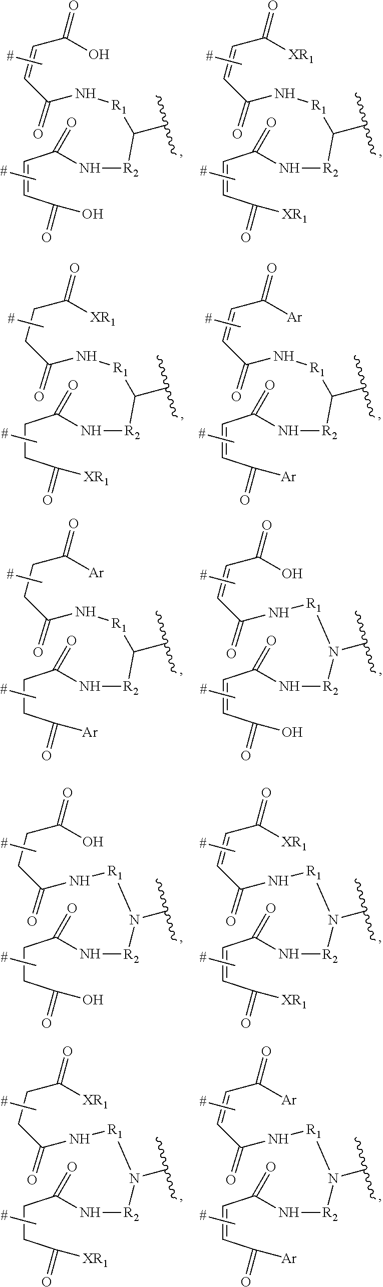

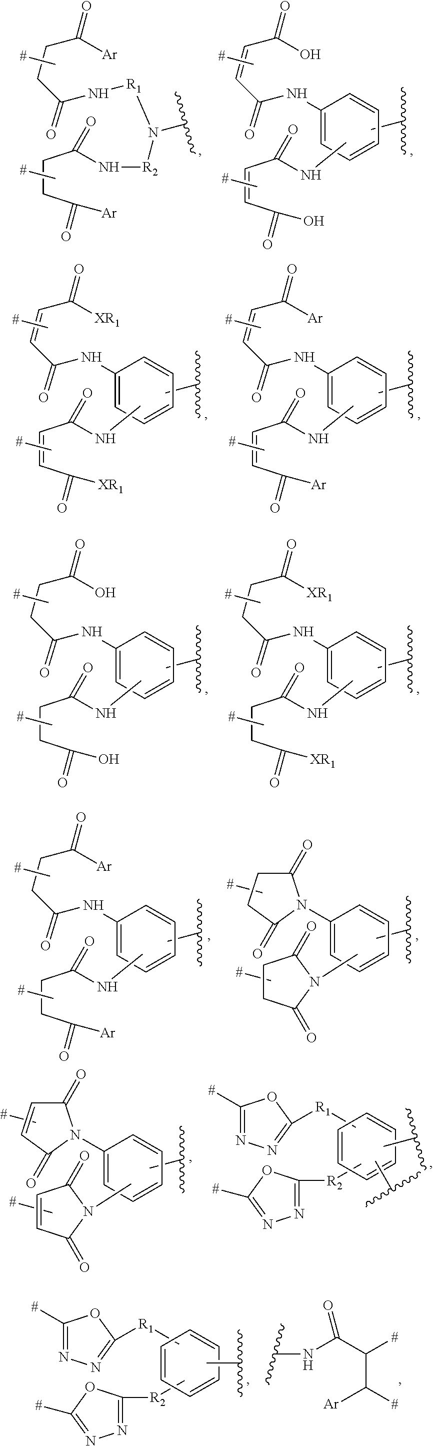

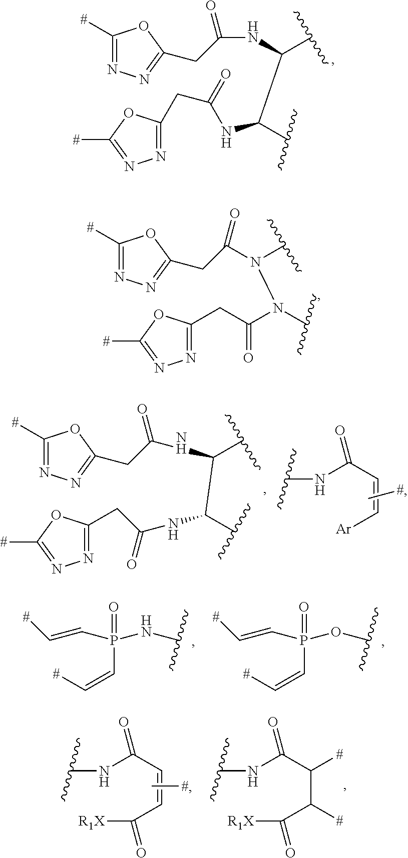

wherein “ [symbol] ” is a site that links to the linker component; “#” is the site as Lv₁′ and Lv₂′ indicated in the formulas that links a S (thiol), O (phenol), NH (amino), CHO (aldehyde), C(═O) (ketone), C(O) (NH) (amide) and C(O)(OH) (carboxylate) of an antibody; wherein R₁, X₁′ and X₂′ are described above; X is O, NH, S, CH₂; the connecting bond “—” in the middle of the two atoms means it can link either one of the two atoms; Ar is an aromatic group; preferably R₁ is H, CH₂, C₂-C₈ of alkyl, ester, amide, Ar, ketone, alkylacid, alkylalcohol, or alkylamine.

More preferably, the following core linker structure (L₁″) having an affinity ligand in Formula (I) (or L₁″ in Formula (Ib′)) as:

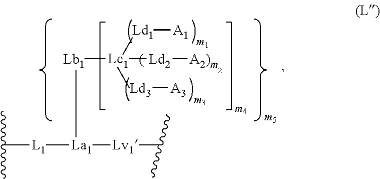

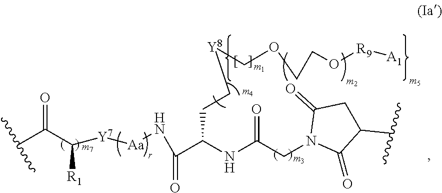

Wherein Aa is L- or D-natural or unnatural amino acids; A₁ is the affinity ligand defined the same above;
R₁ is H, C₁-C₈alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0, 1, 2, 3, 4, 5, 6, 7, or 8; when r is not 0, (Aa) r is the same or different amino acids or peptide units; when r=0, (Aa) r is absent;
m₁=1-18 (0, 1, 2, 3, 4, 5, 6, . . . , 18); m₂=1-100 (1, 2, 3, 4, 5, 6, 7, 8, . . . , 100); m₃ or m₅=1-8 (1, 2, 3, 4, 5, 6, 7, or 8); m₄=0-8 (0, 1, 2, 3, 4, 5, 6, 7, or 8);
Y₇ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC6H₄NH, CH₂;
Y₈ is NHC(═O), NH, O, NHS(O₂), NH(SO), NHS(O₂) NH, NHP(O)(OH)NH,

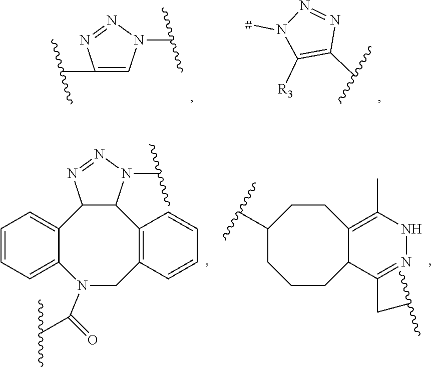

C(O)O, C(O), OC(O)NH, C(O)NH, or Ar;
R₉ is (O═)CR₁, (O═)CNHR₁, NHC(═O), NH, O, NHS(O₂), NH(SO), NHS(O₂)NH, NHP(O)(OH)NH or C(O)NH, R₁ (COCH₂NH)ₘ₄H, R₁(Aa)ᵣ, (Aa)r, C(O), Ar or

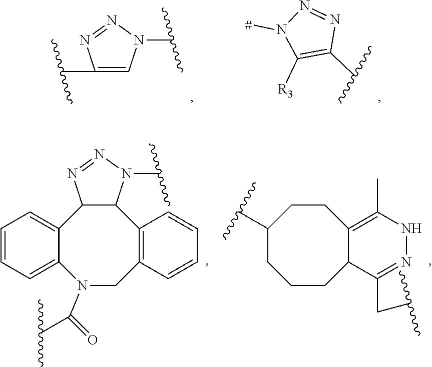

wherein R₃ is H, C₁-C₈ alkyl, ester, amide, Ar, ketone, alkyl acid, alkyl alcohol, alkyl amine, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂; R₁ is defined above.

Or the following core structure (called L₁″ and L₂″ fused jointly) in Formula (III) (or the L₁″ and I₂″ fused in Formula (IIIb′) accordingly);

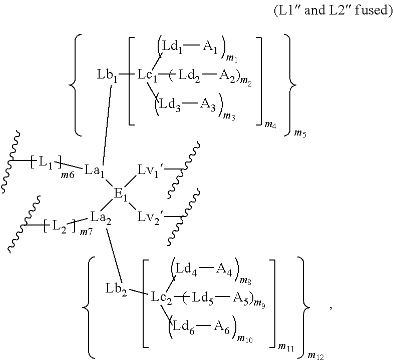

Which is preferably the following formula (Ib′) and (Ic′):

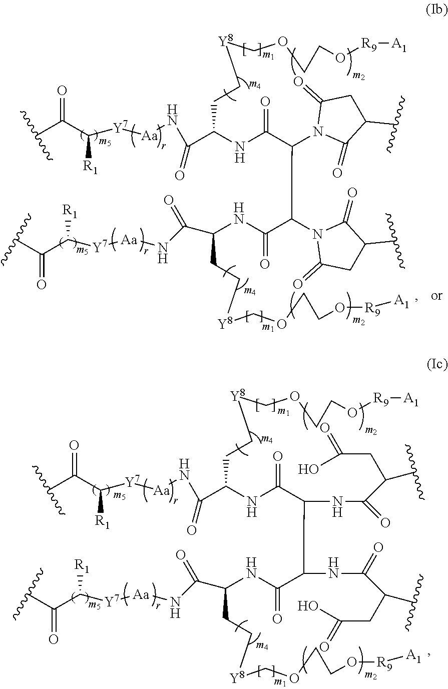

Wherein R₁, Y₇, Y₈, R₉, A₁, Aa, r, m₁, m₂, m₄, and m₅ are defined the same above.

In certain embodiments, the examples of the conjugates of formula (I), (II), (III) and (IV) are illustrated below:

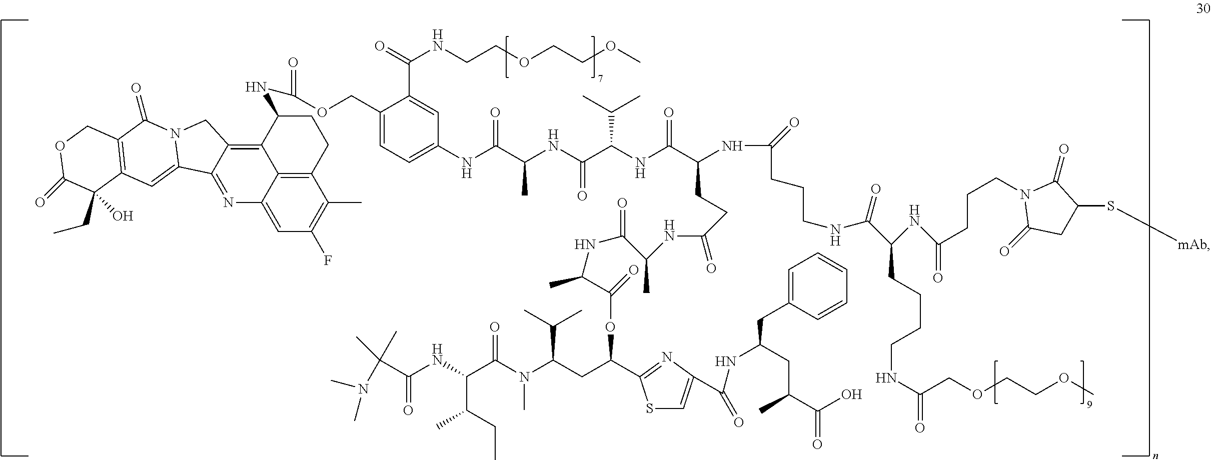

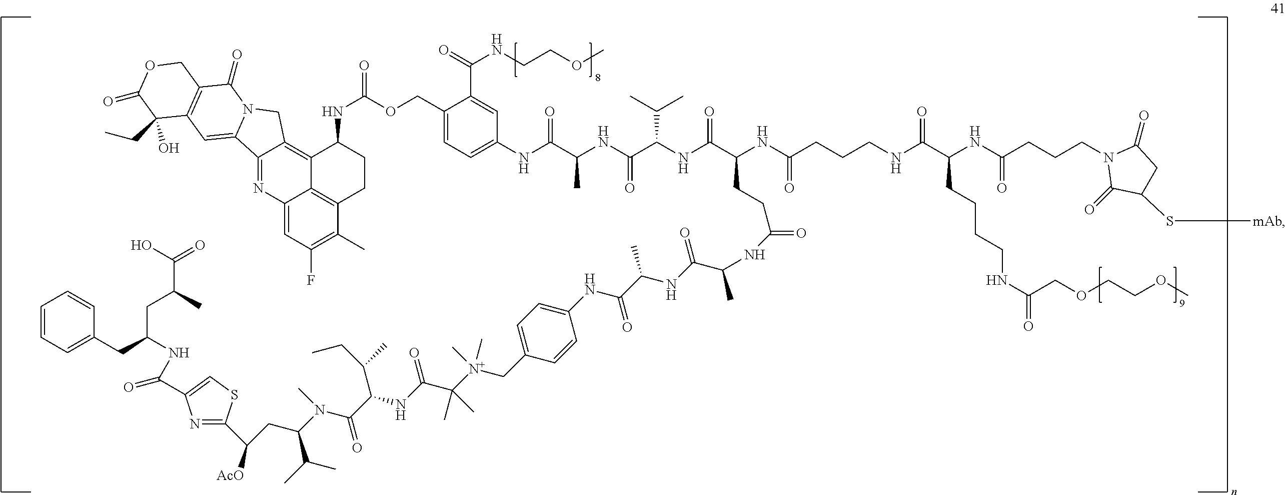

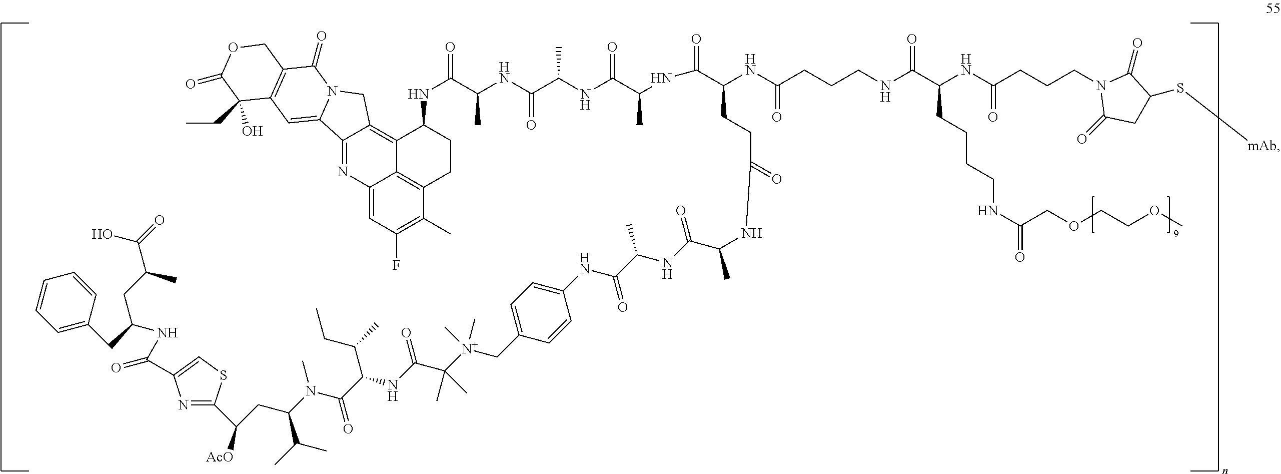

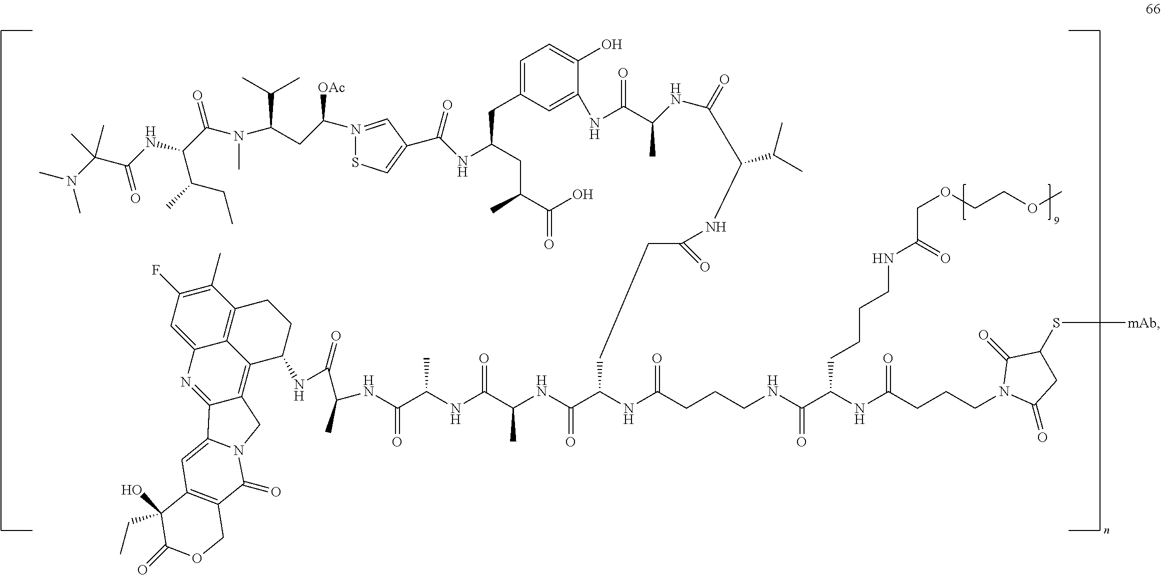

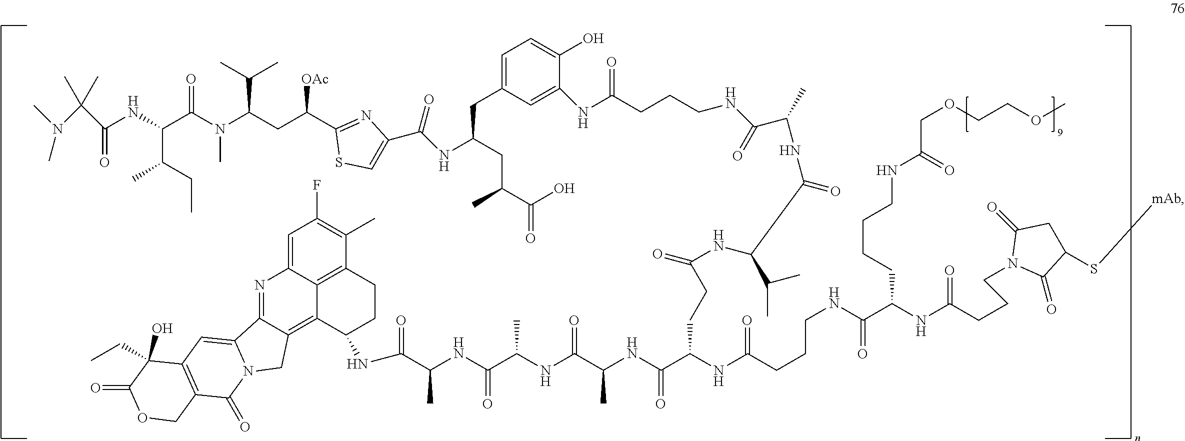

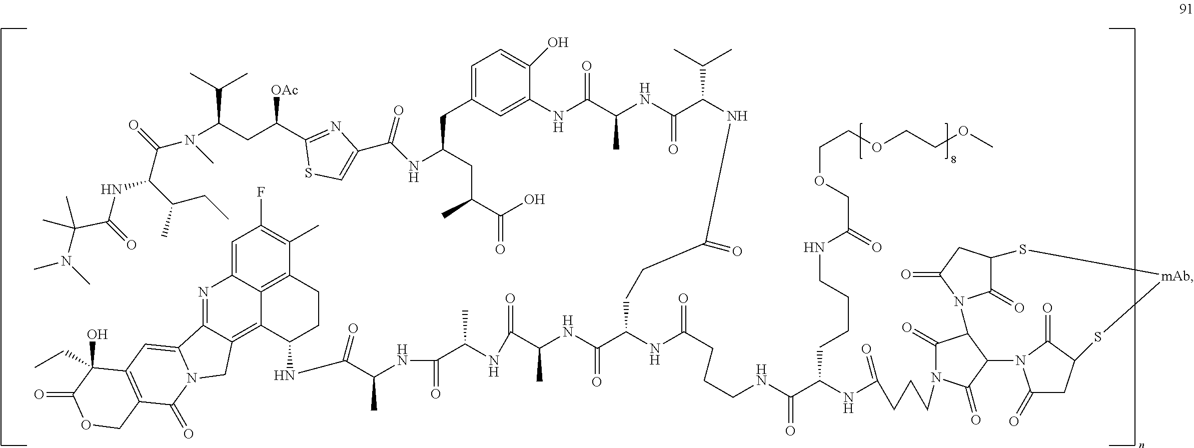

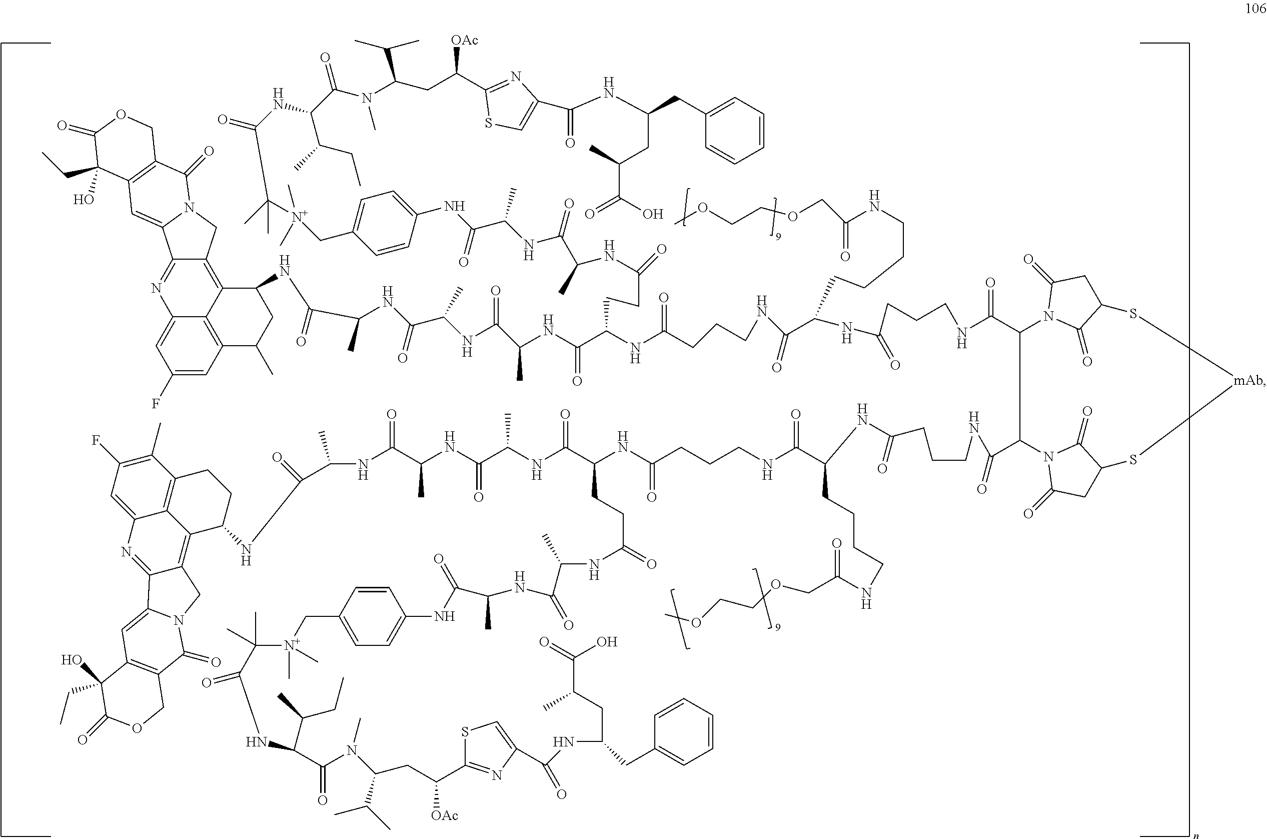

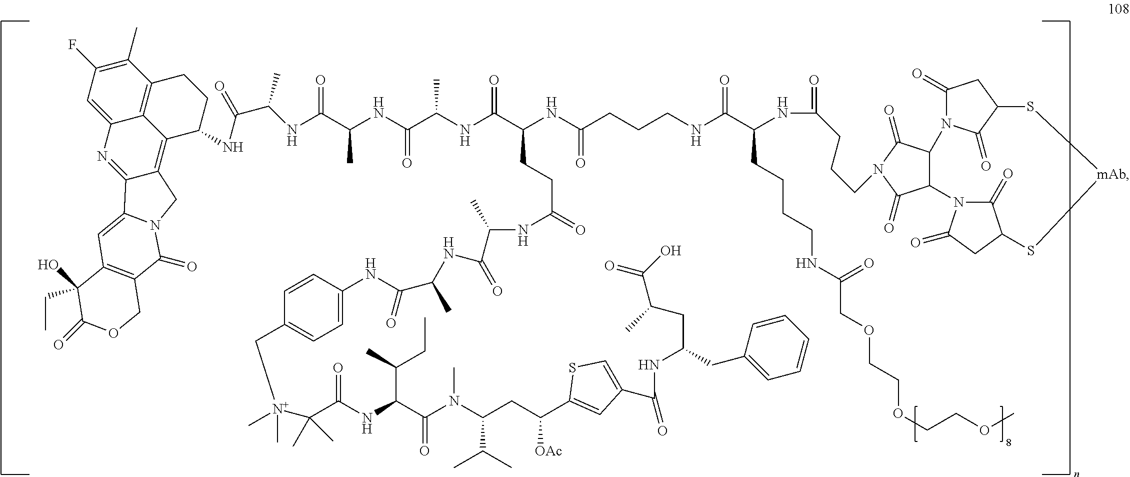

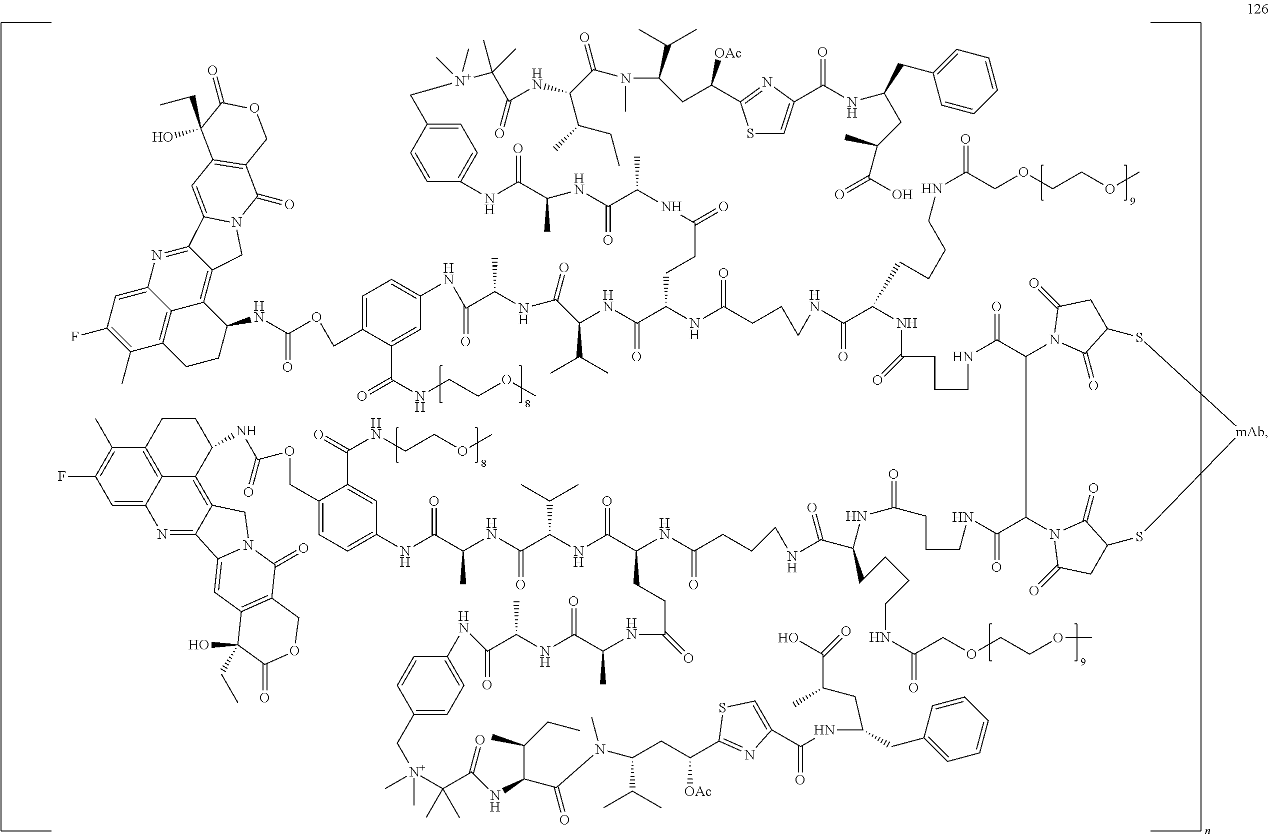

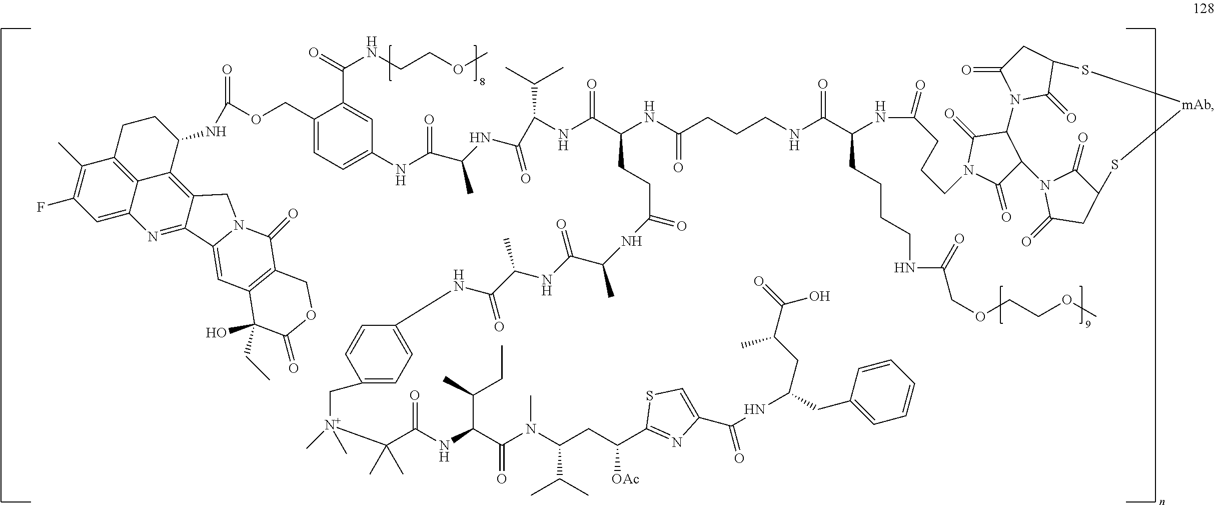

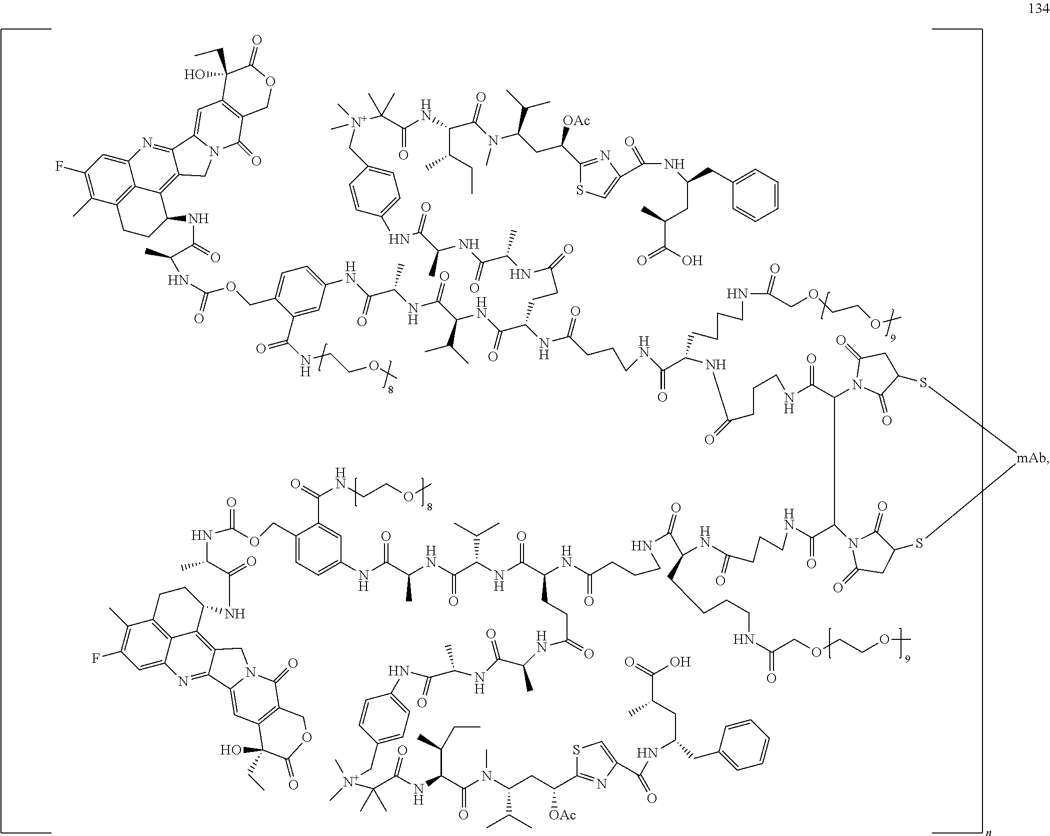

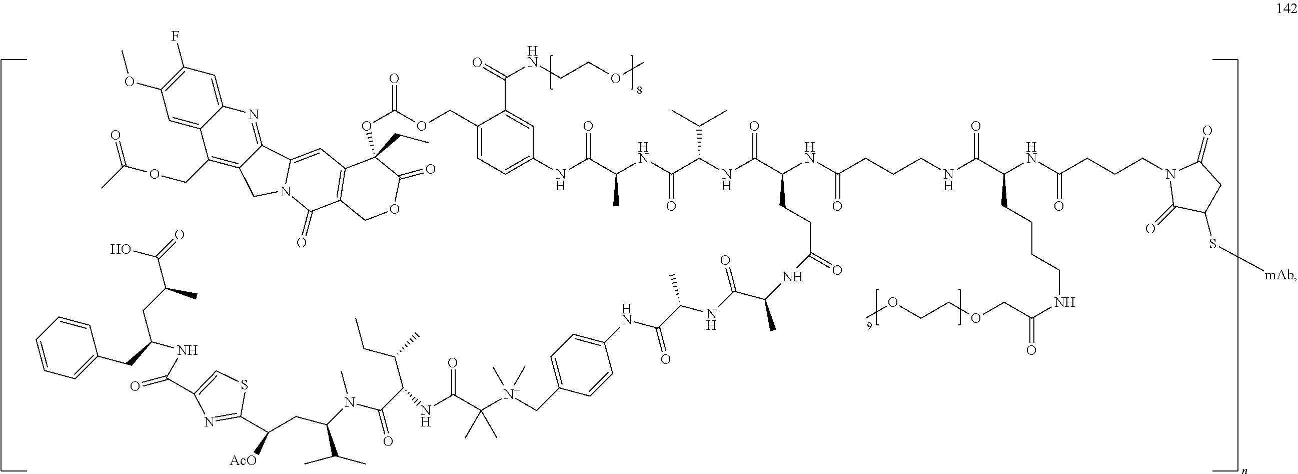

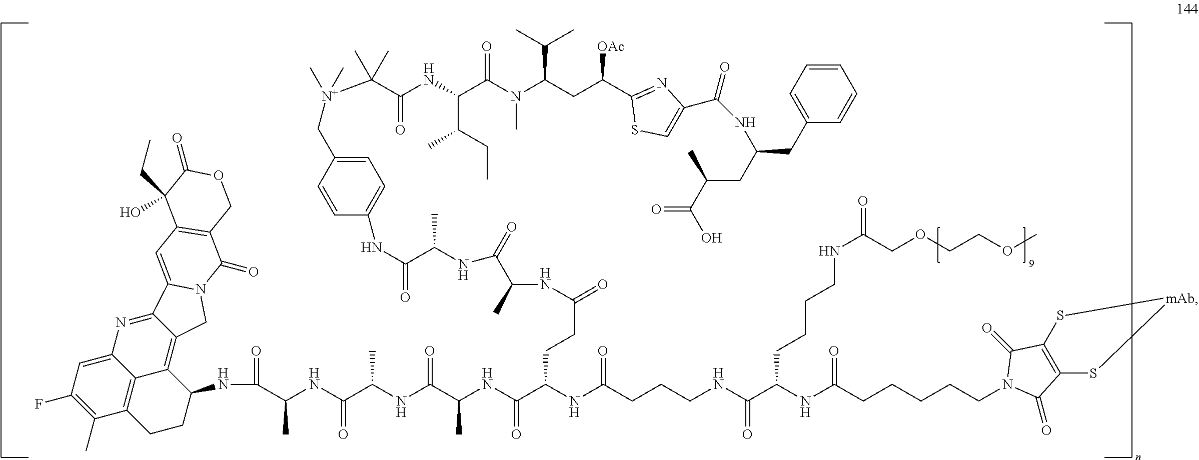

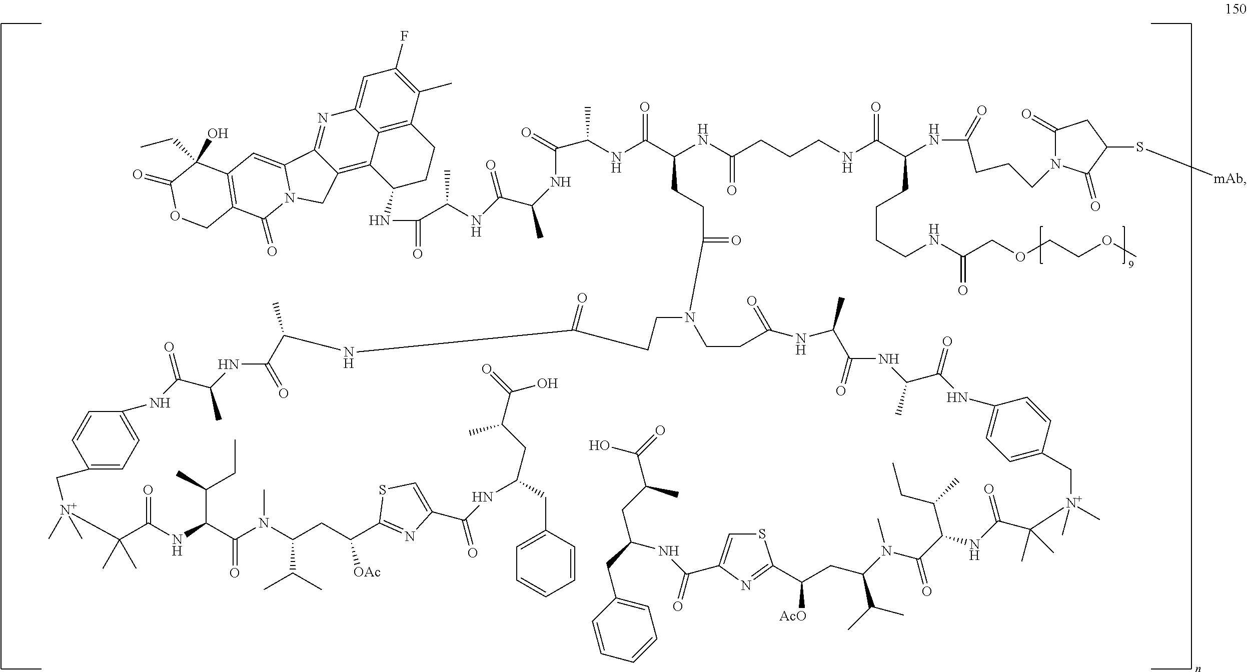

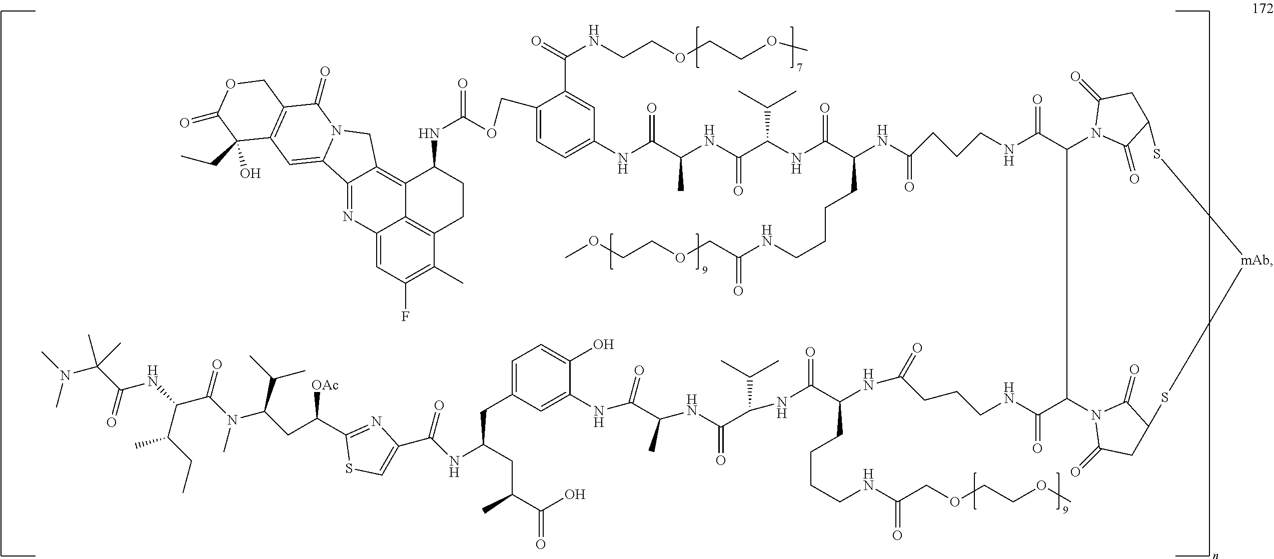

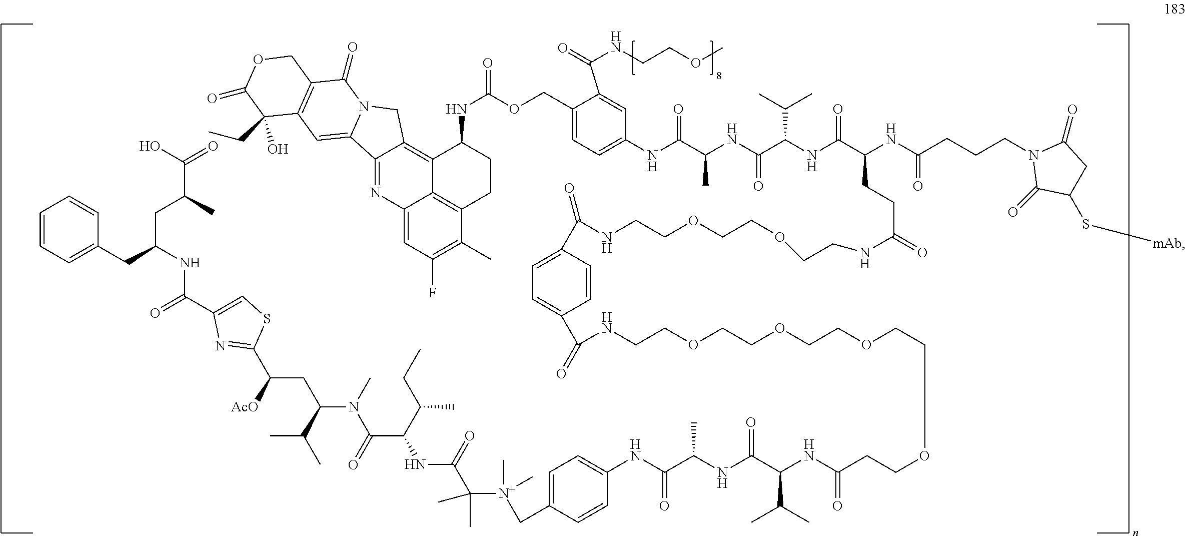

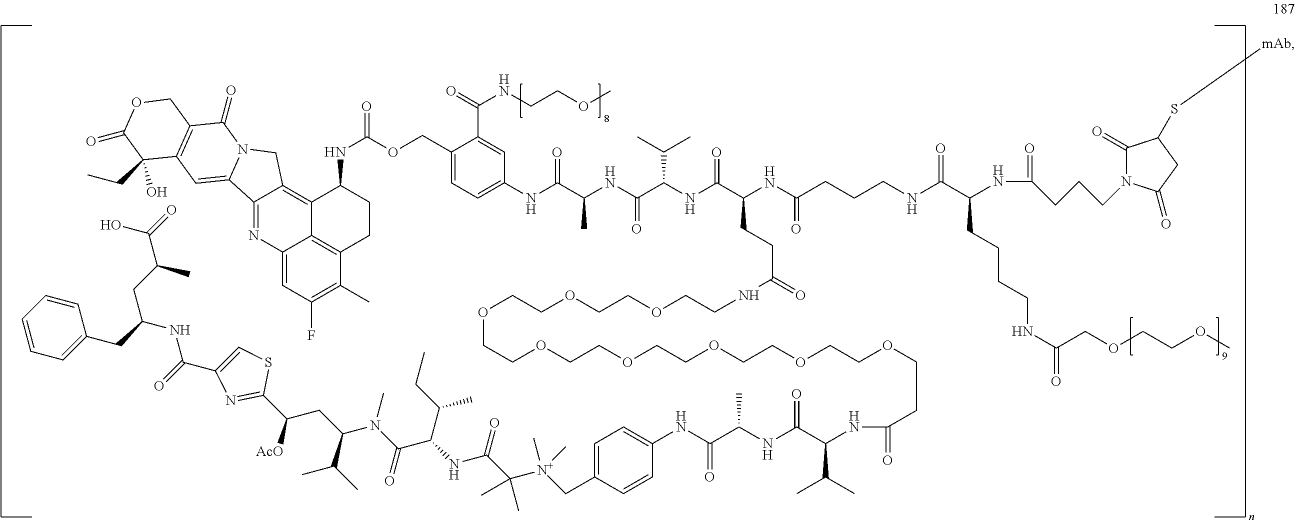

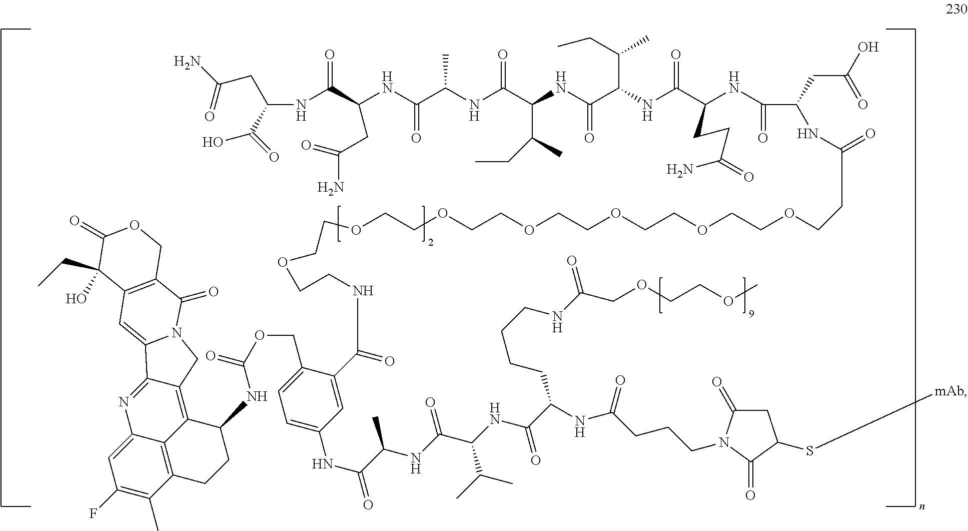

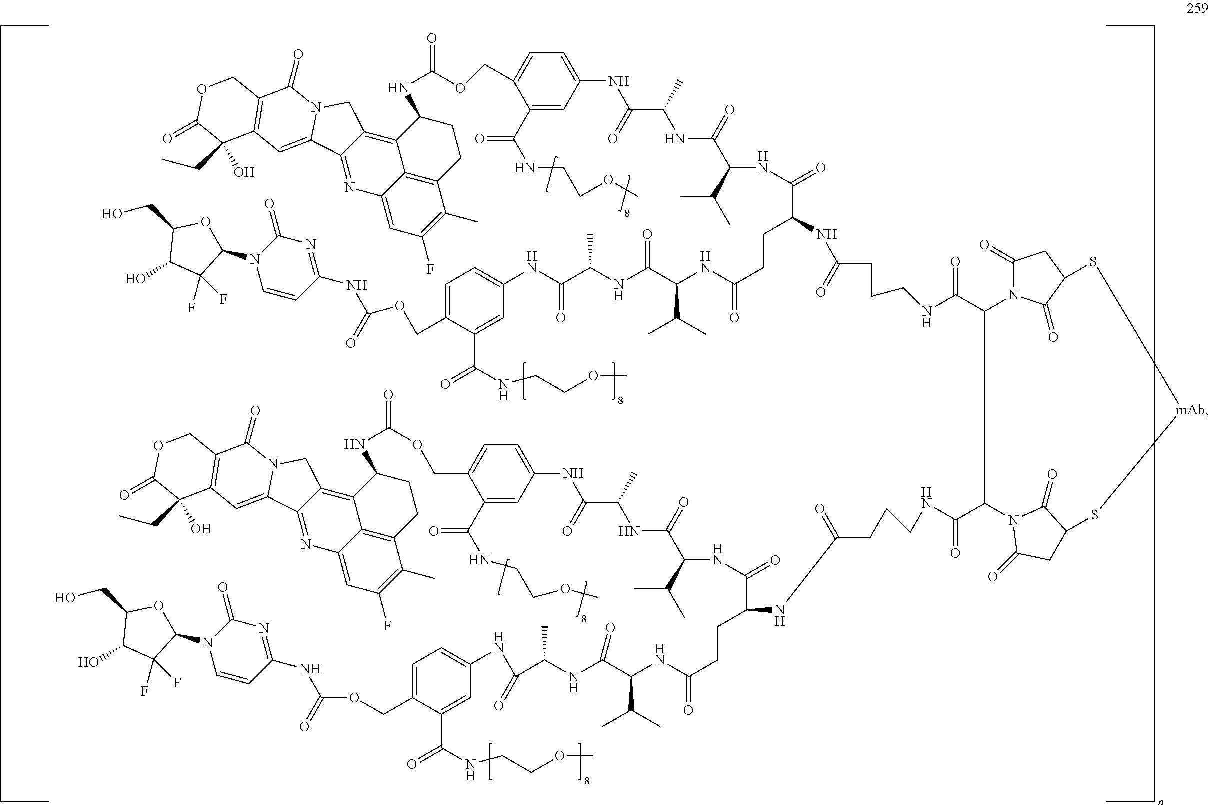

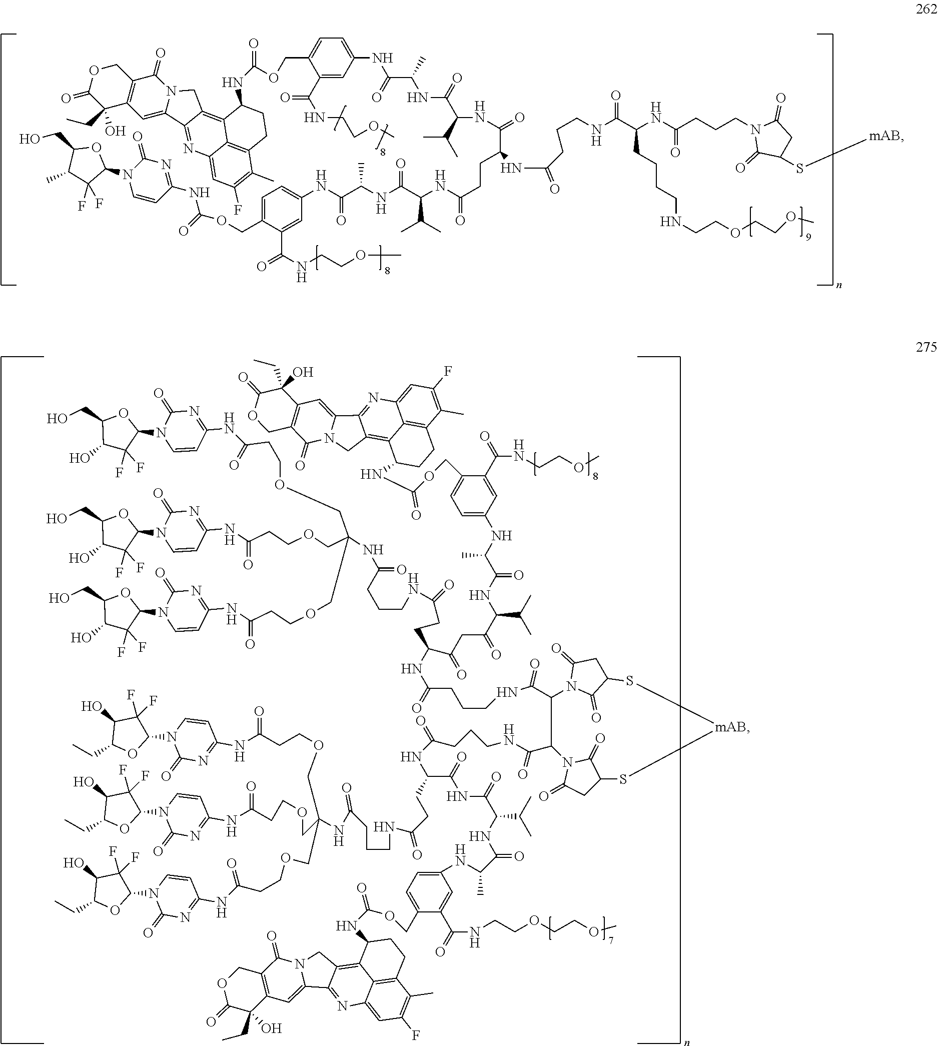

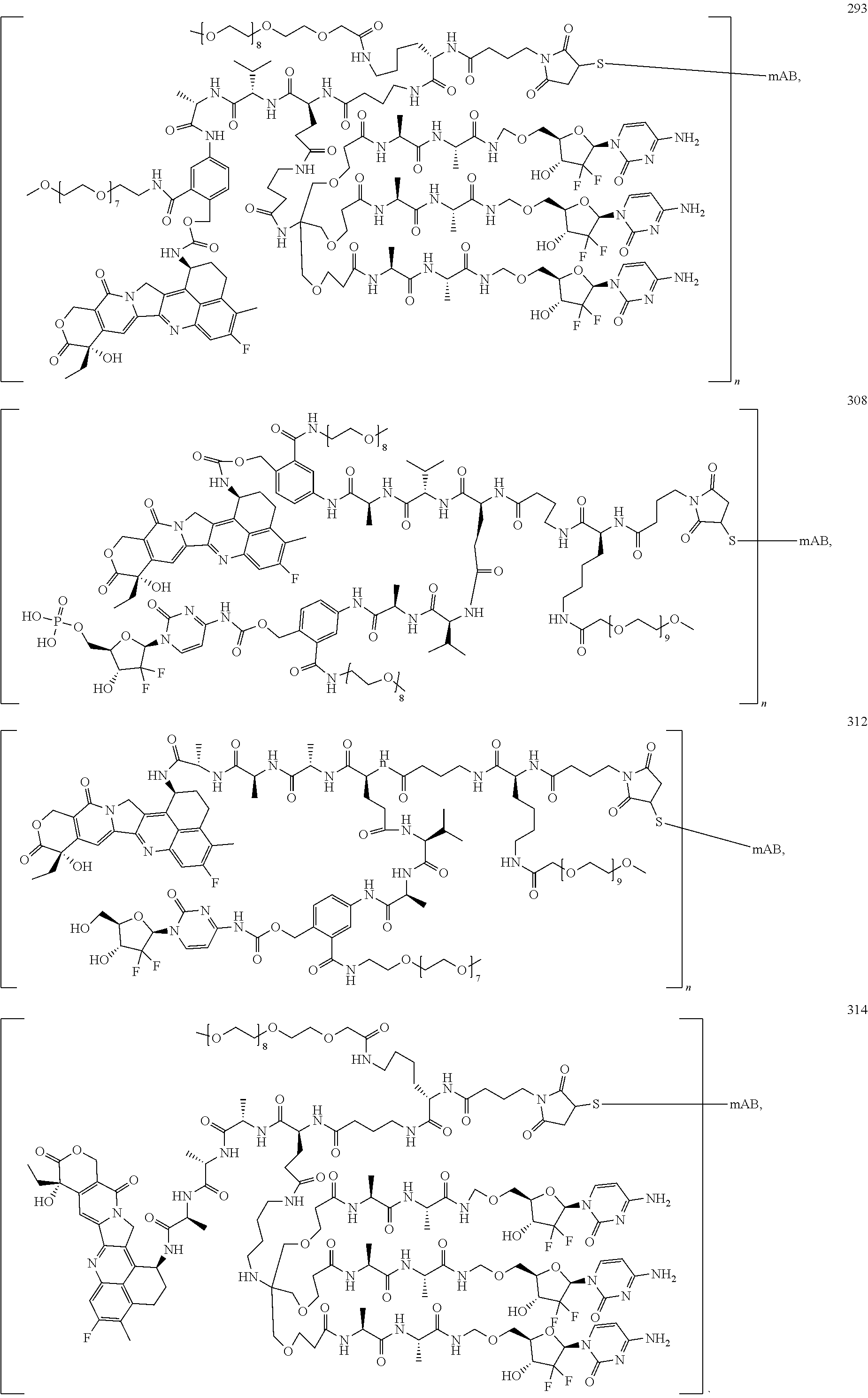

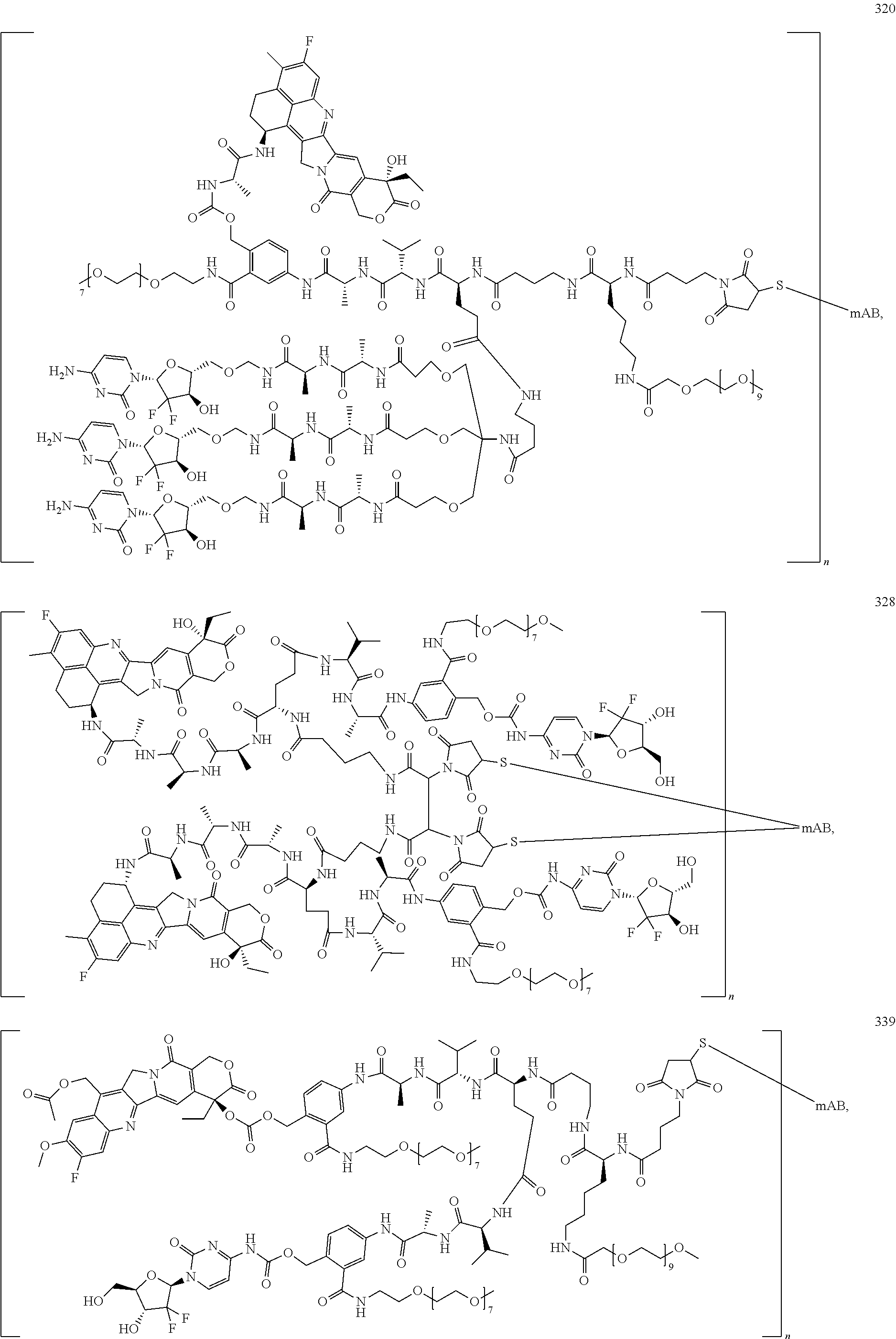

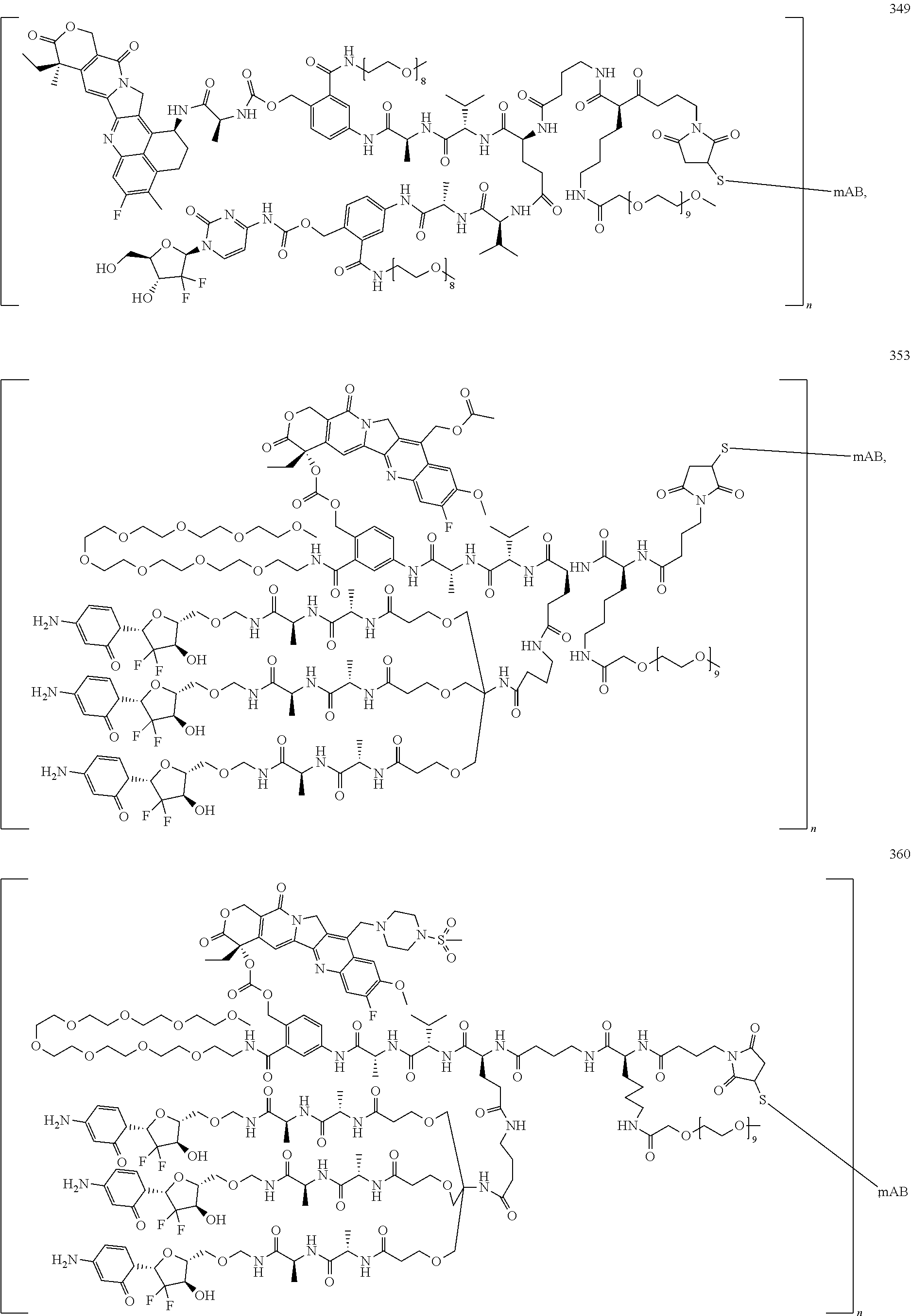

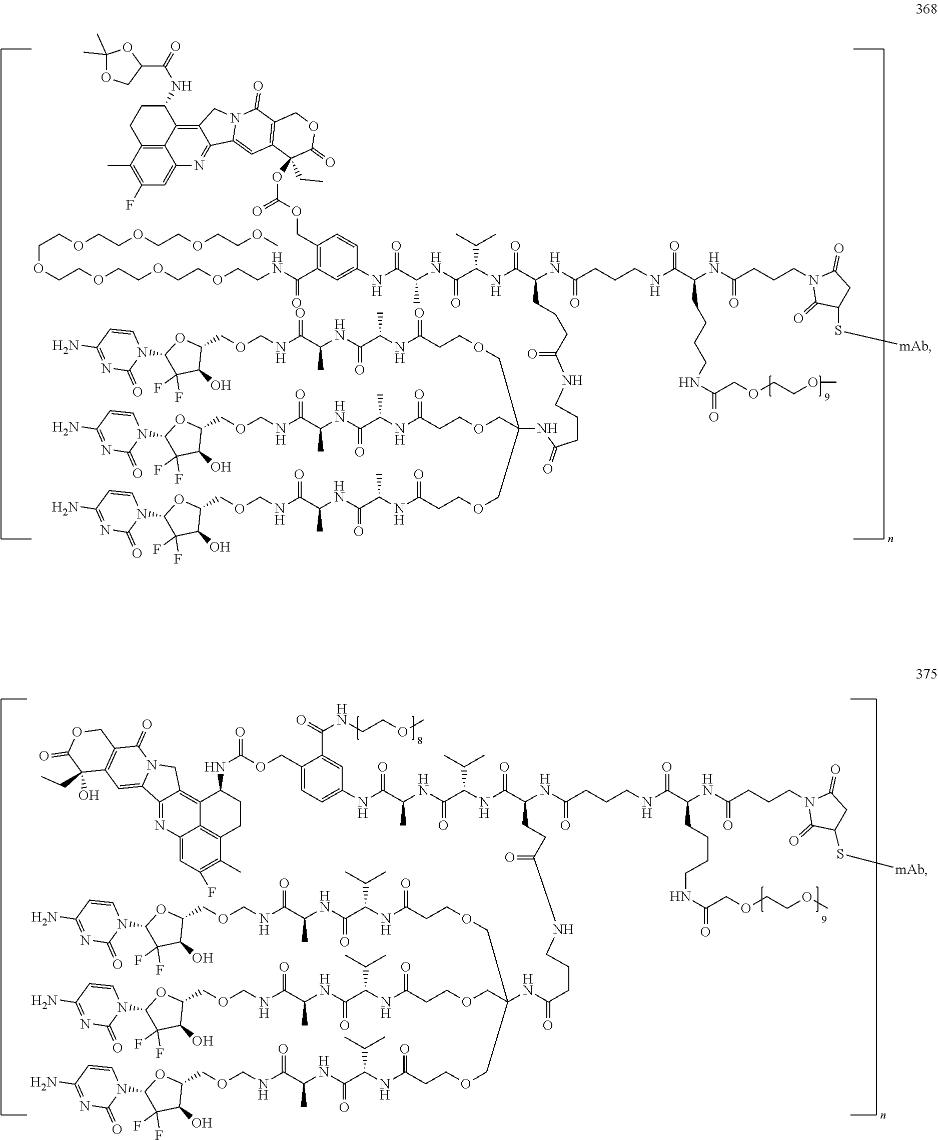

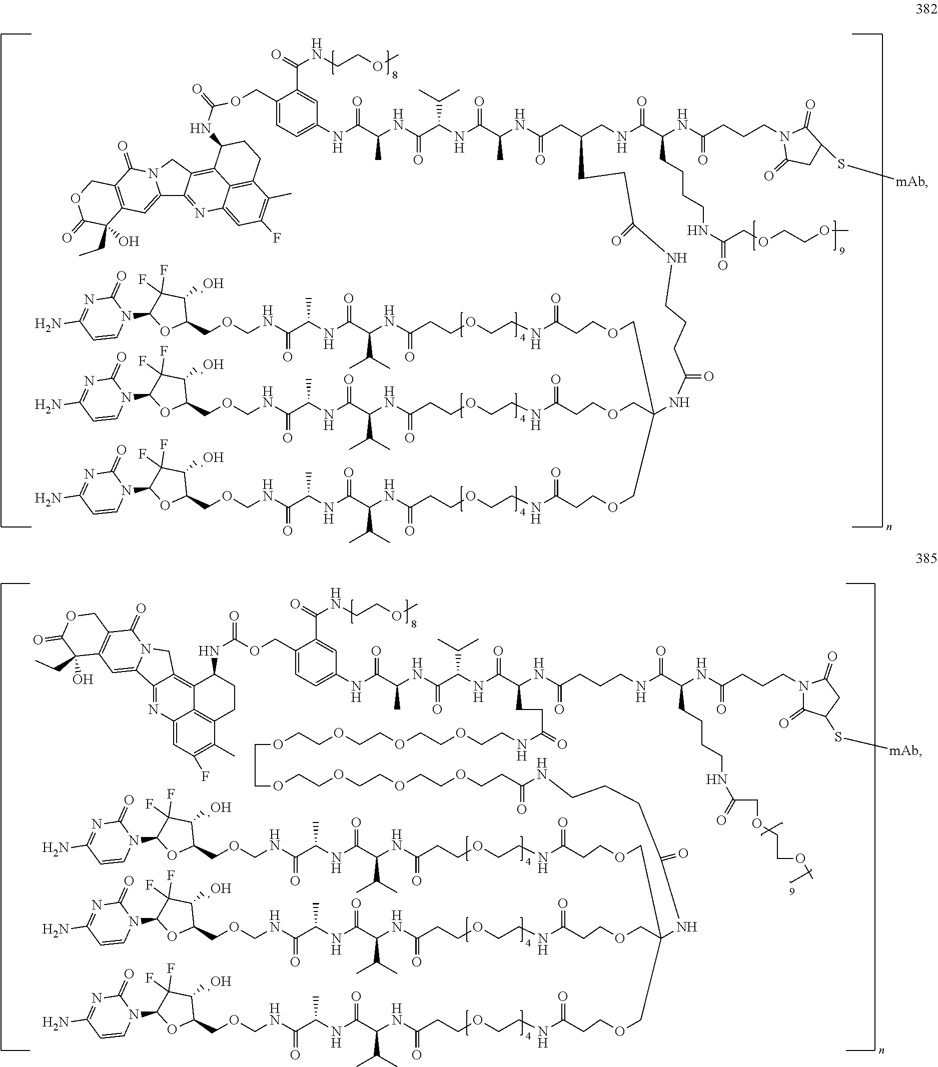

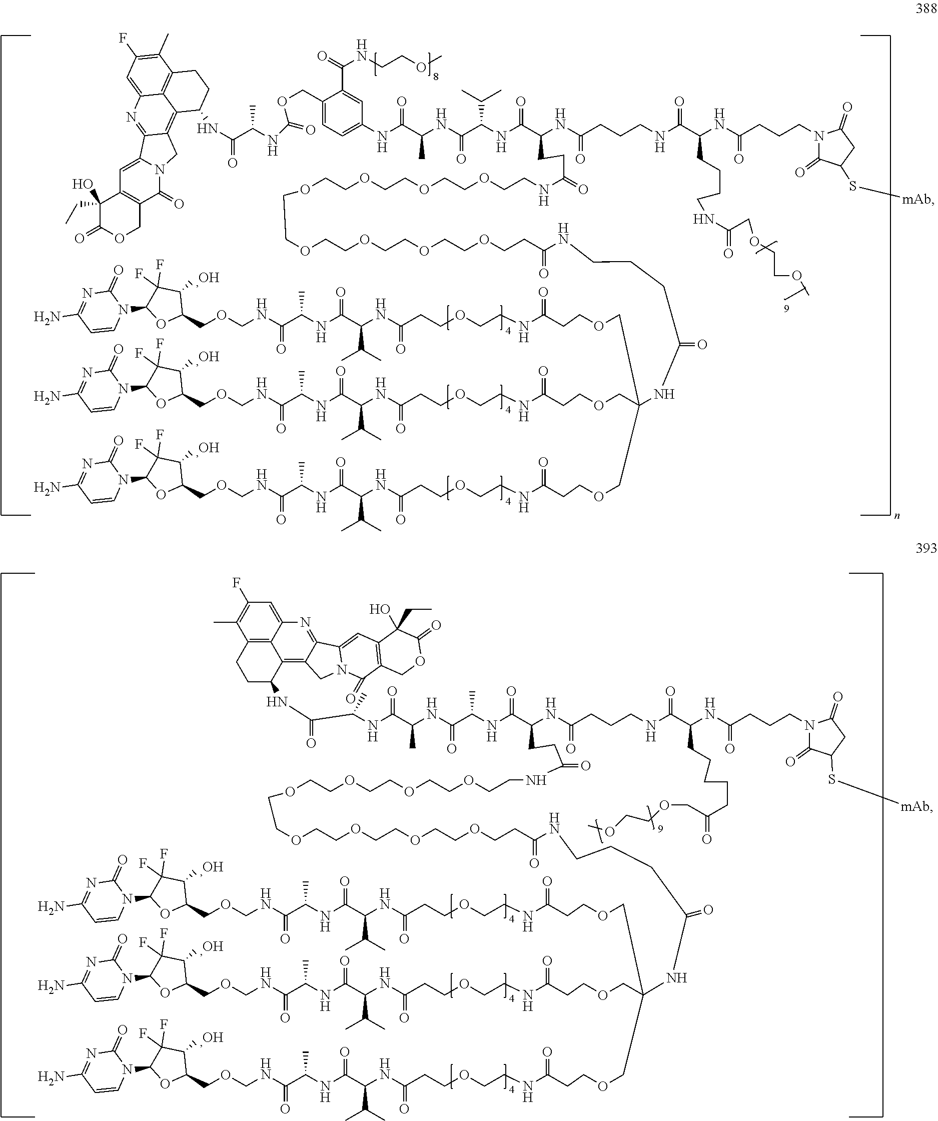

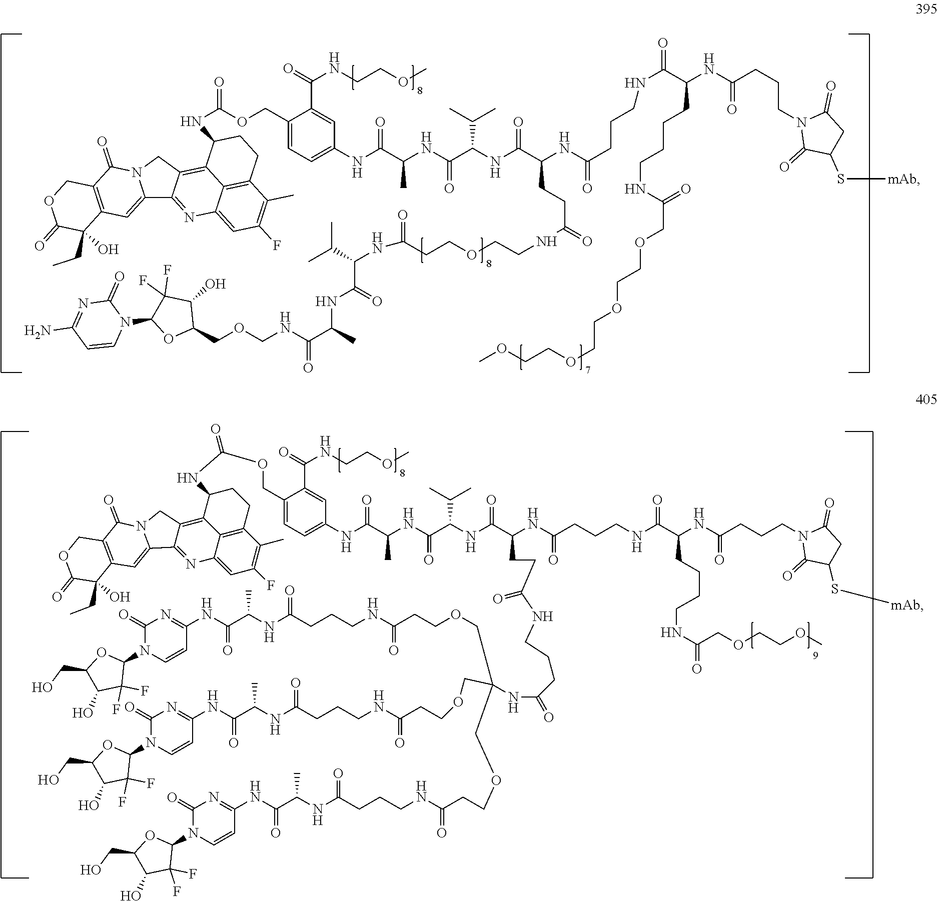

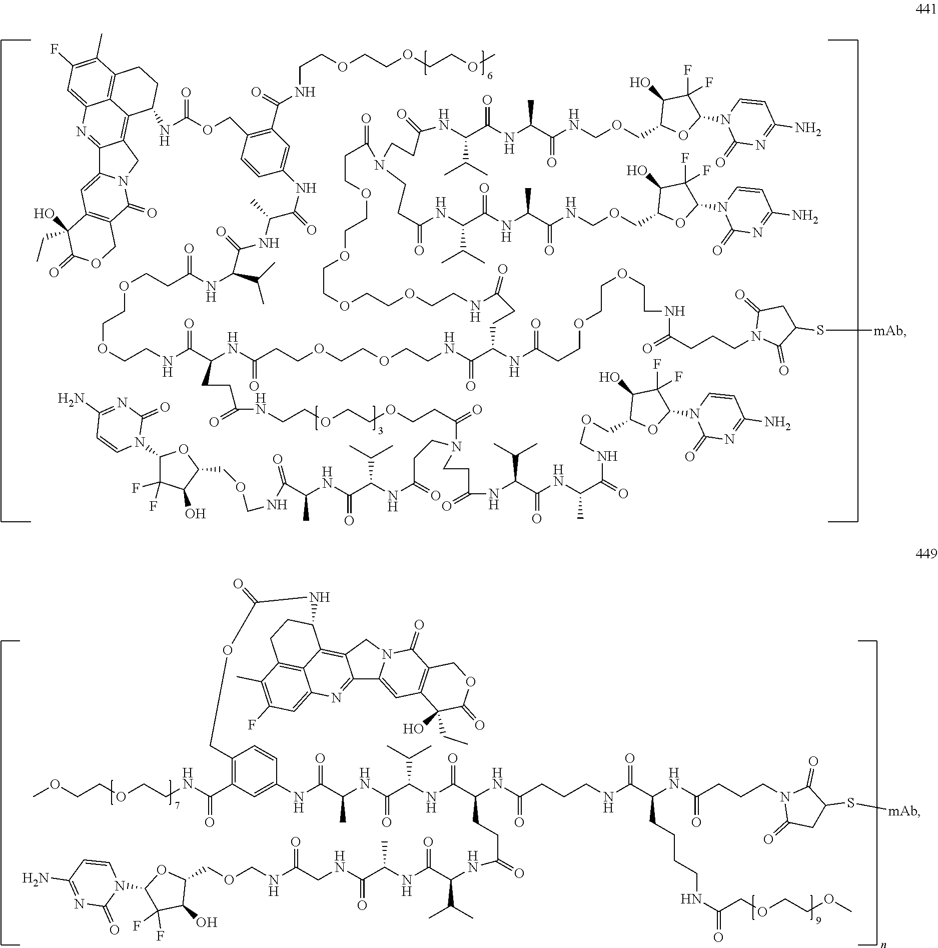

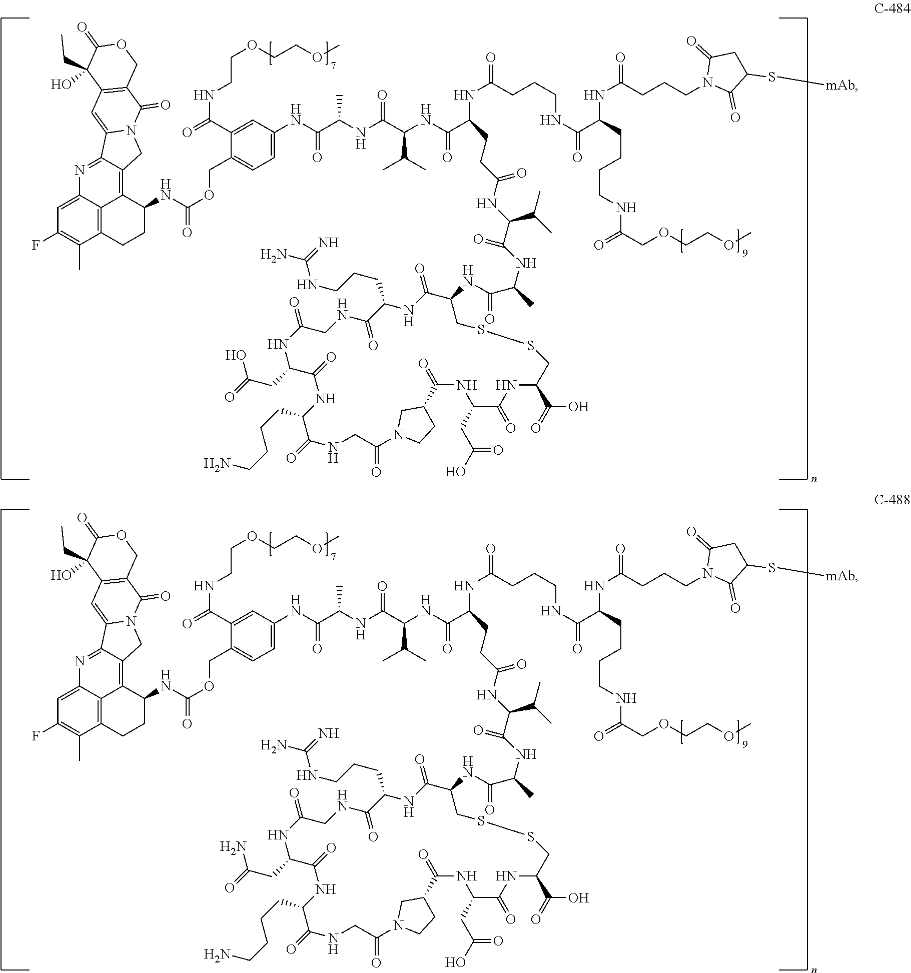

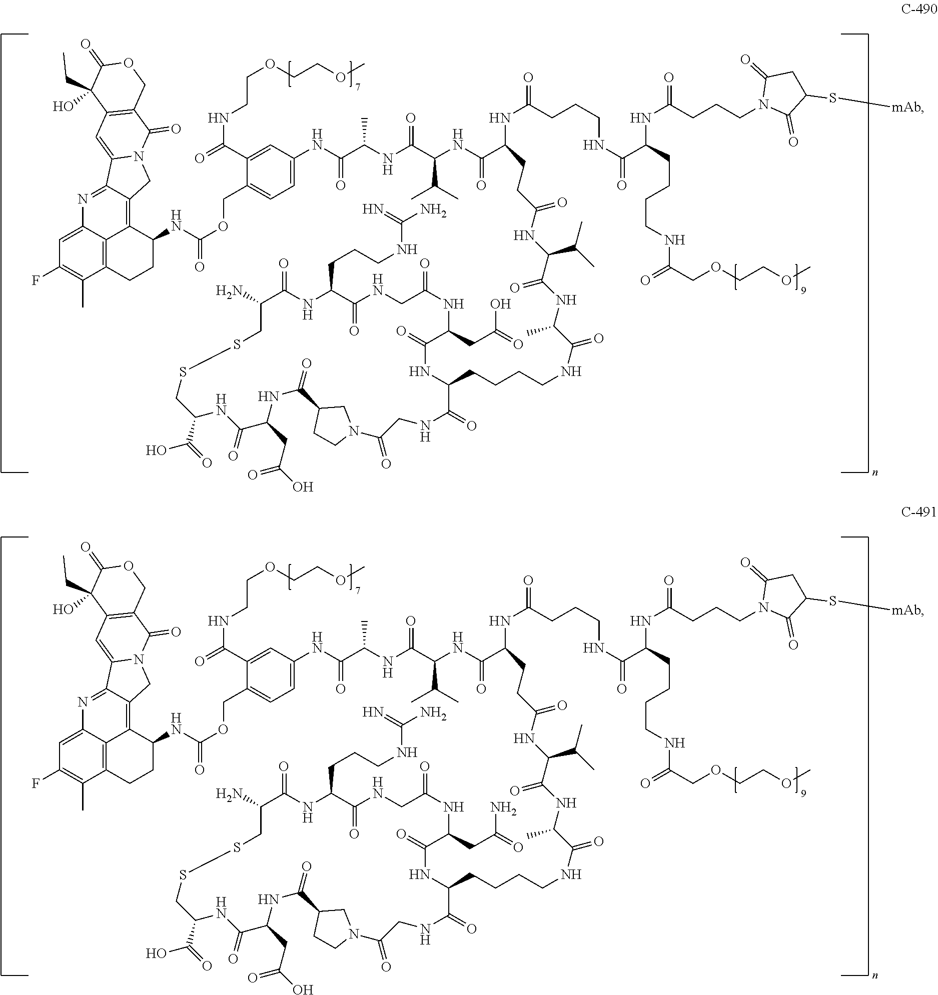

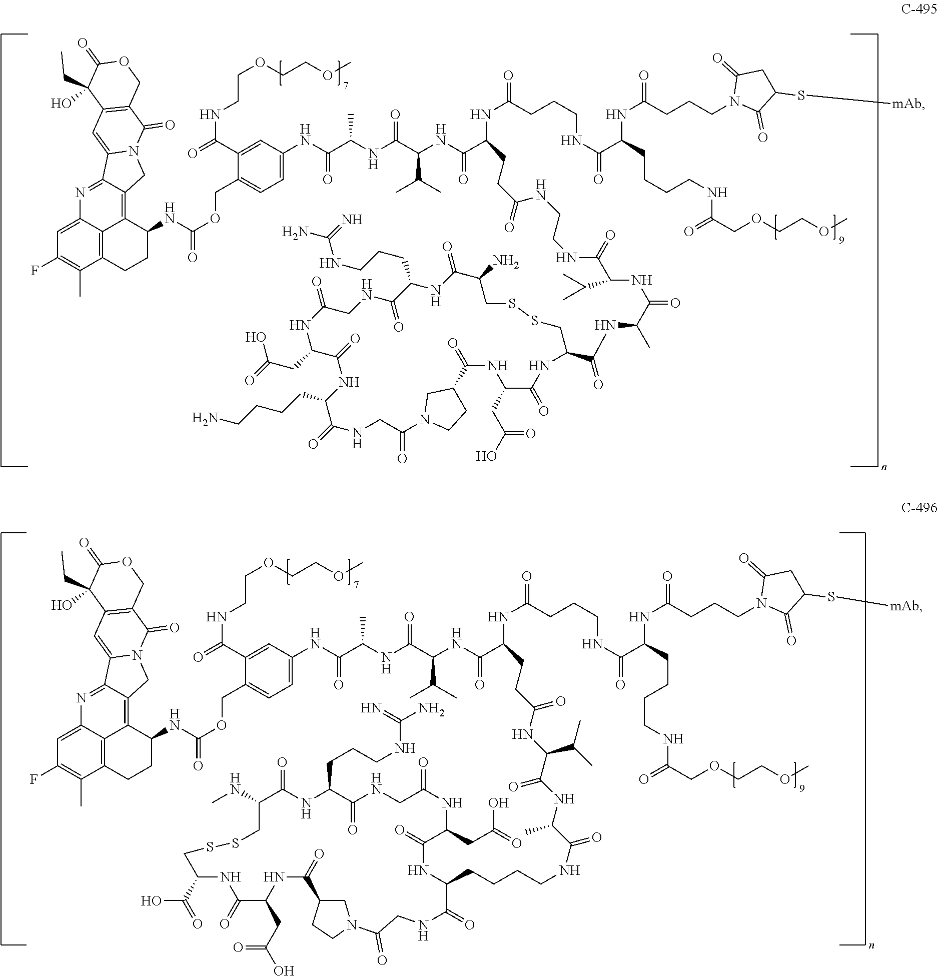

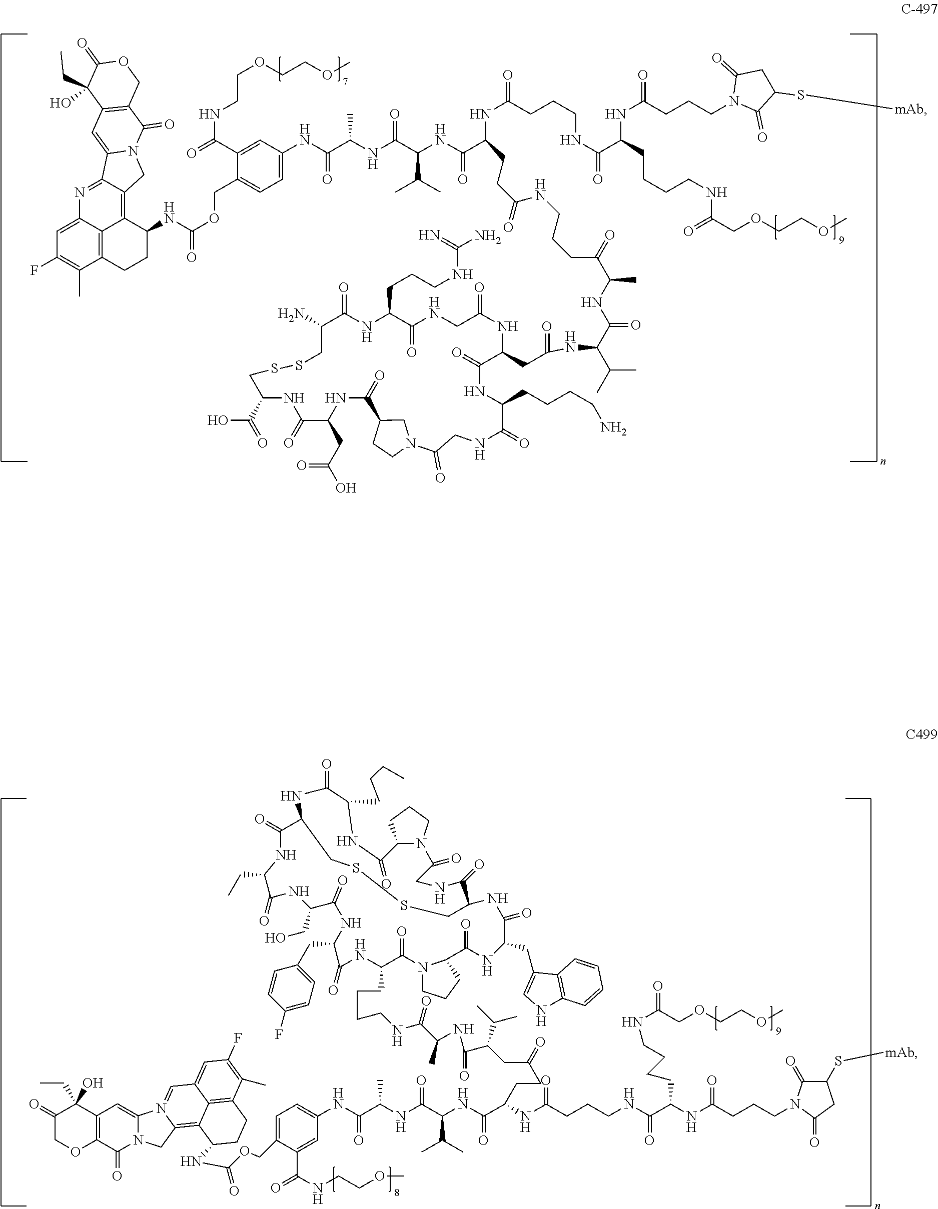

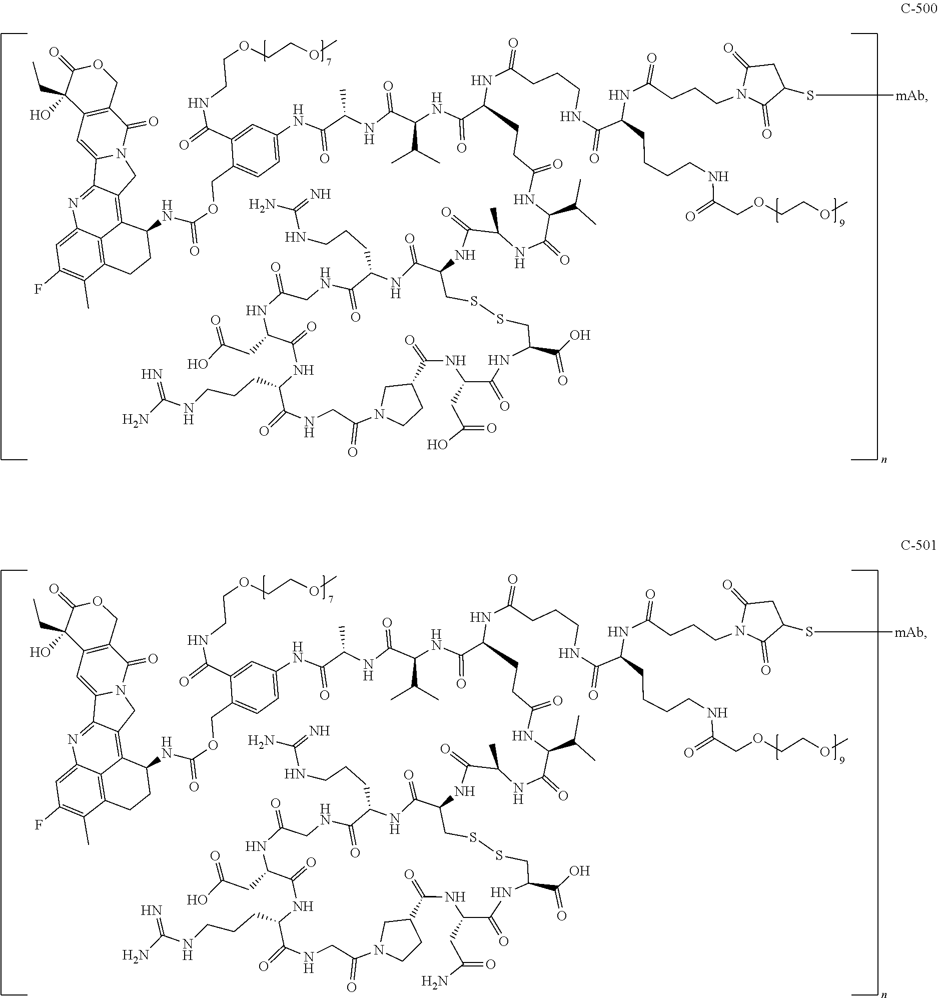

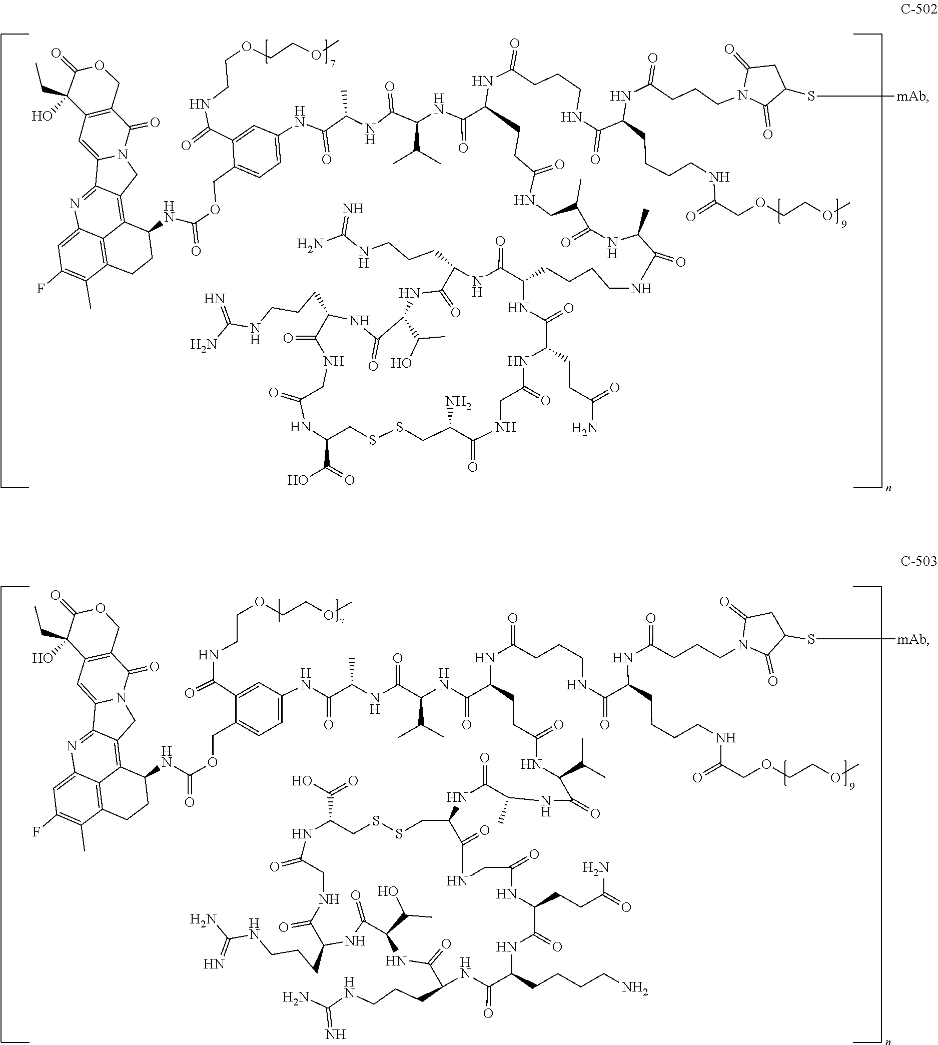

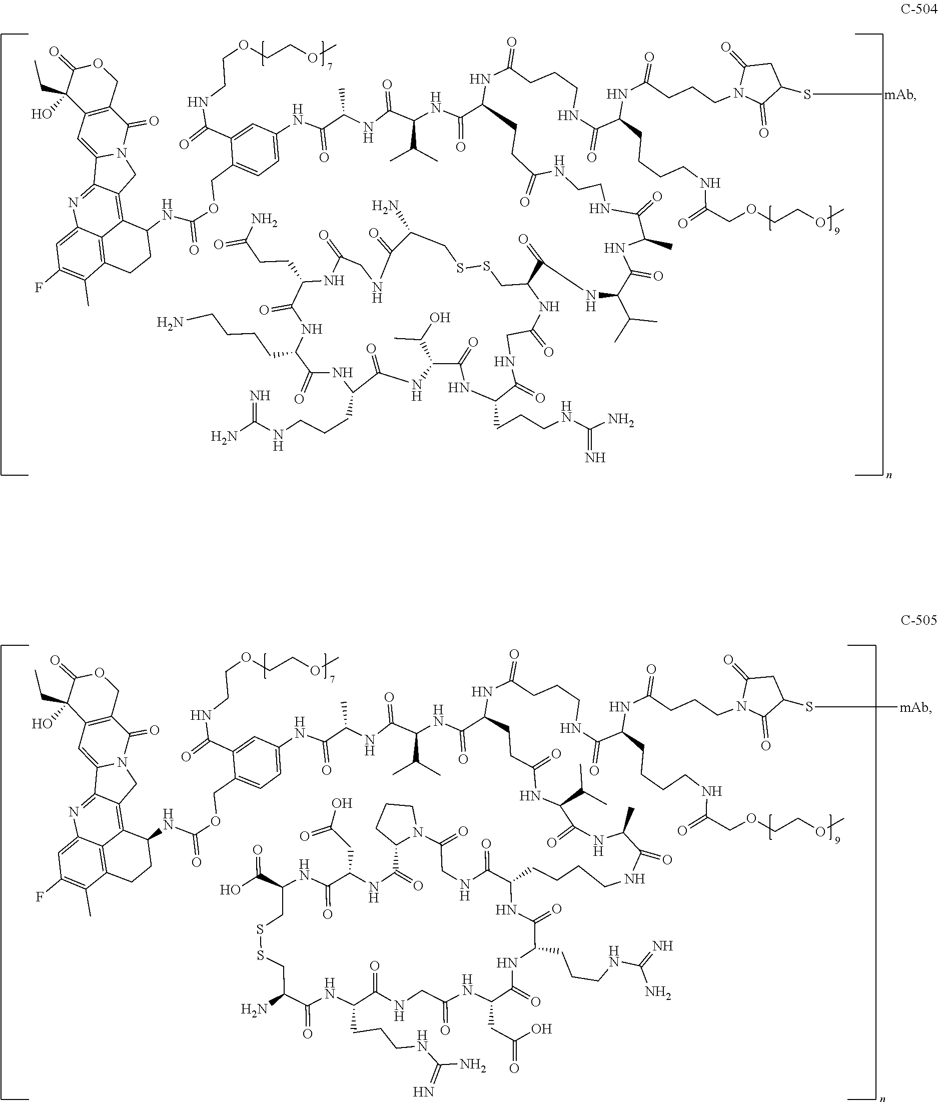

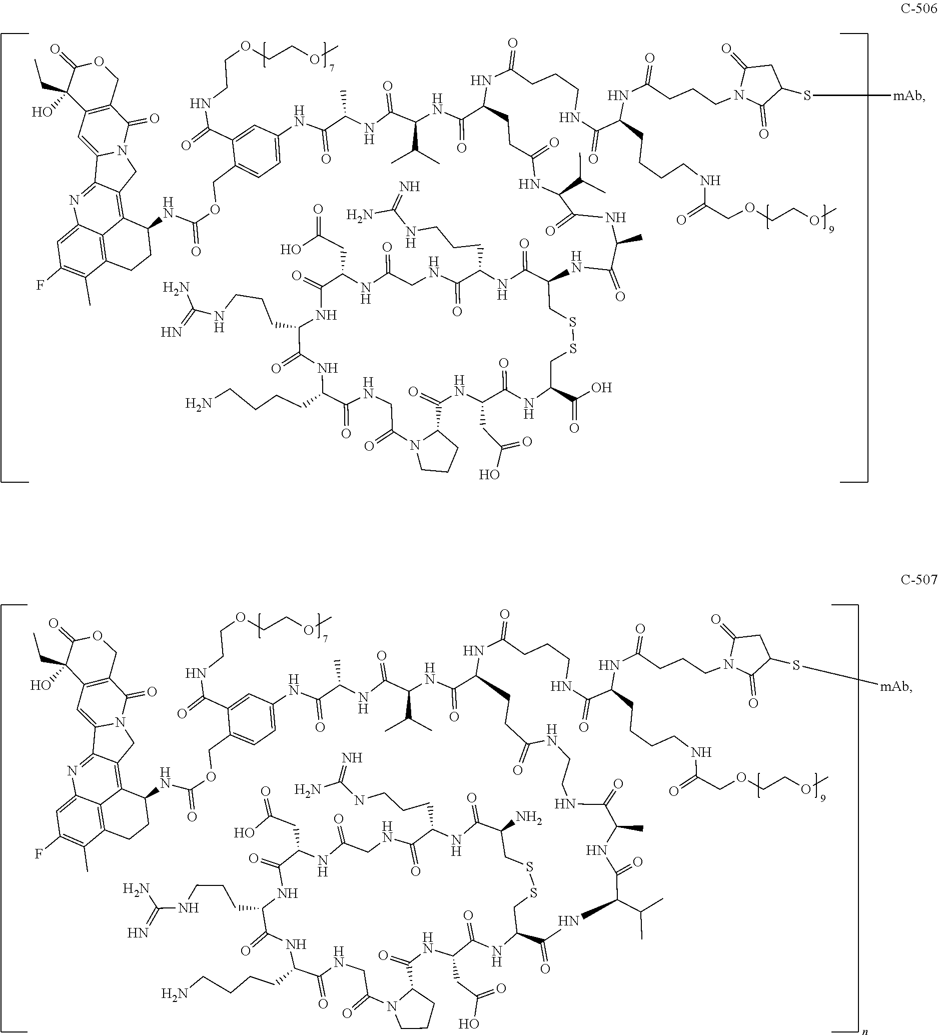

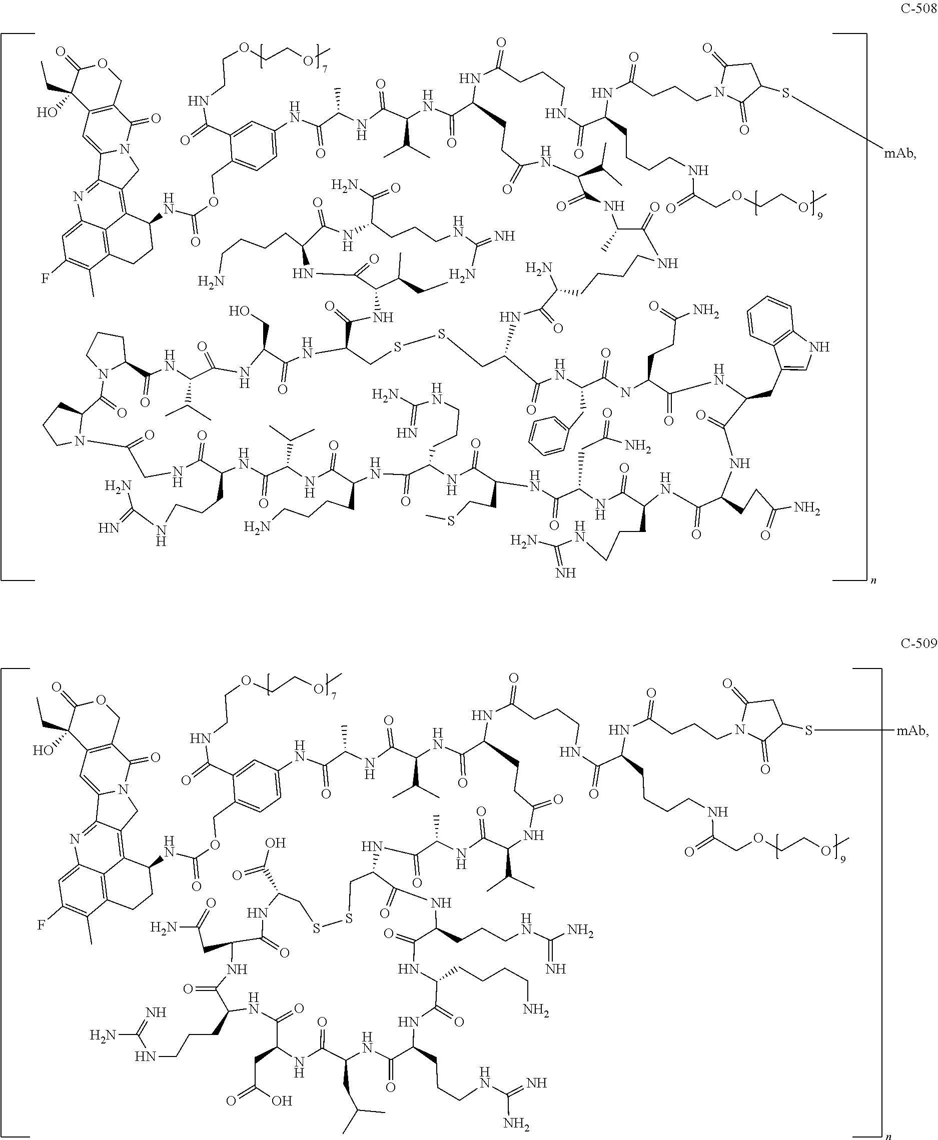

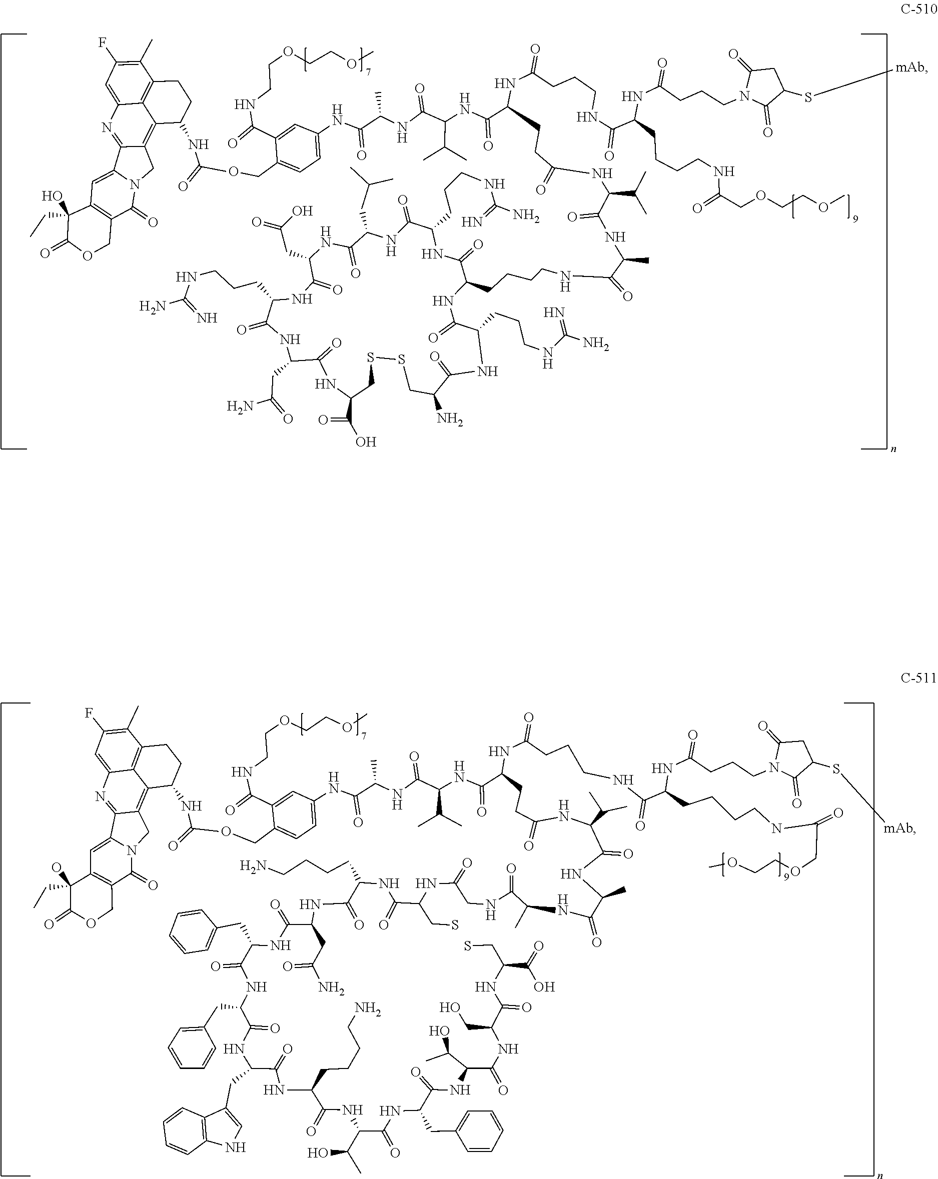

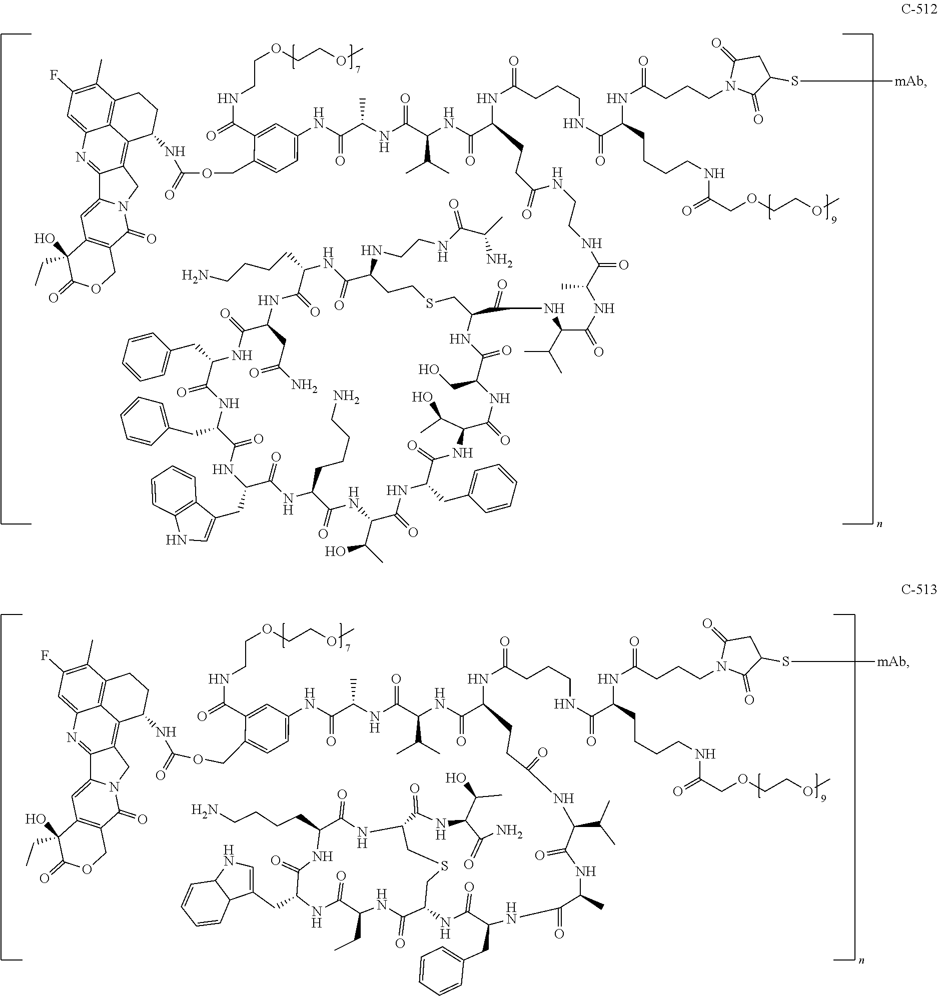

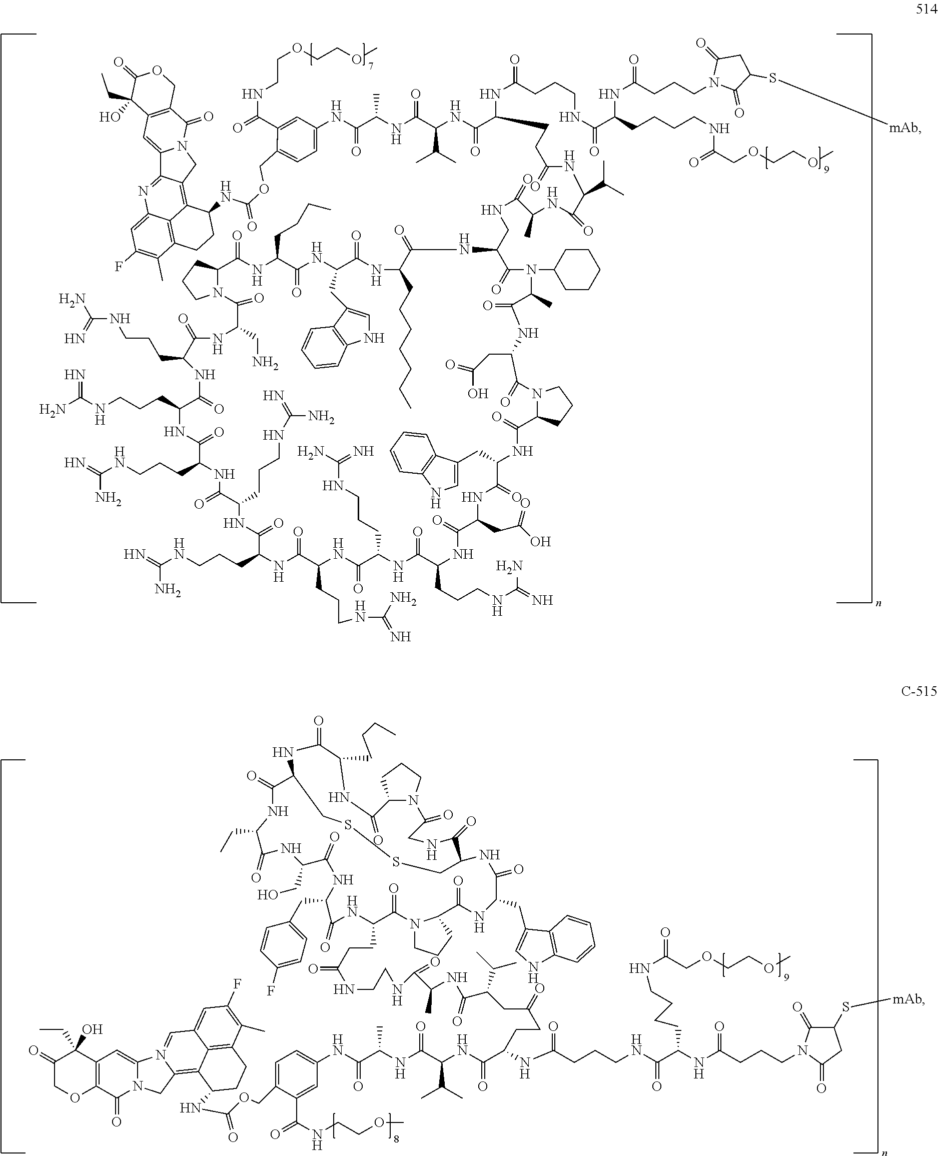

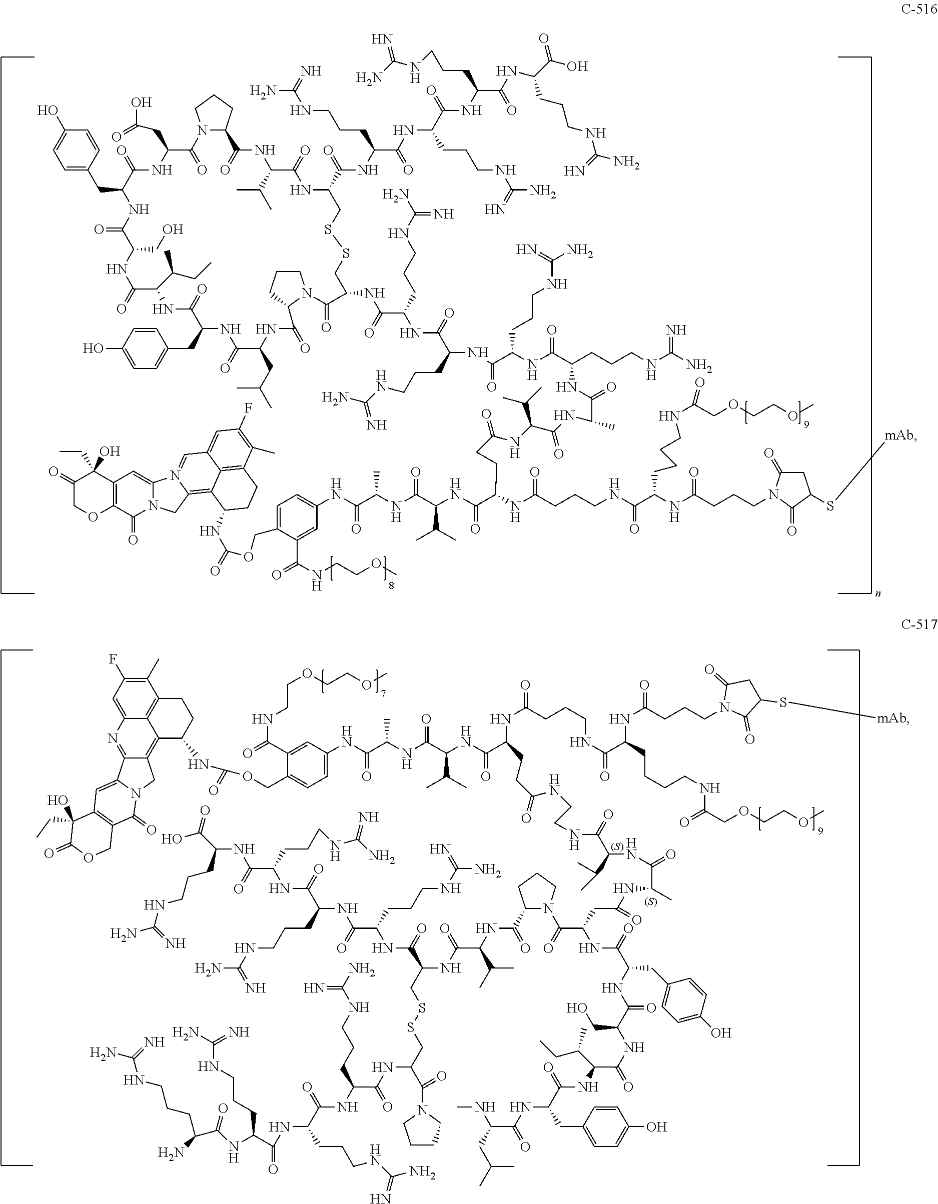

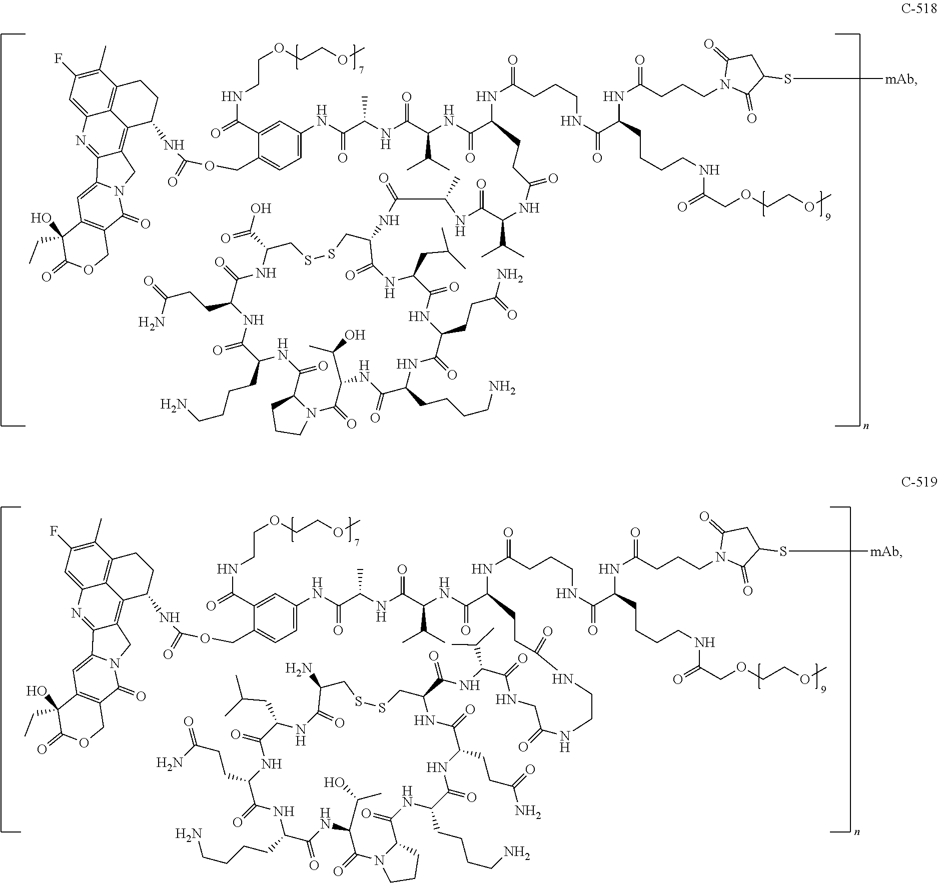

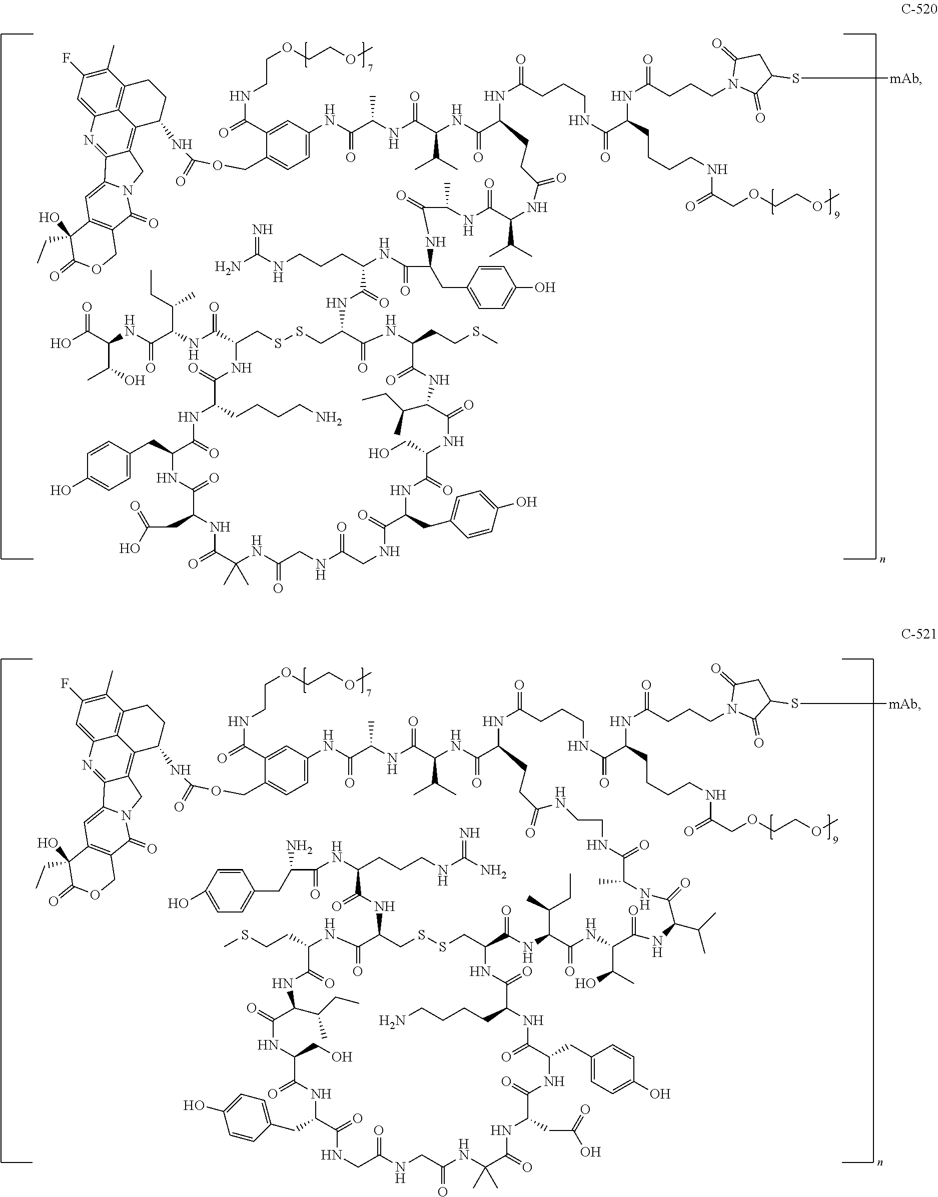

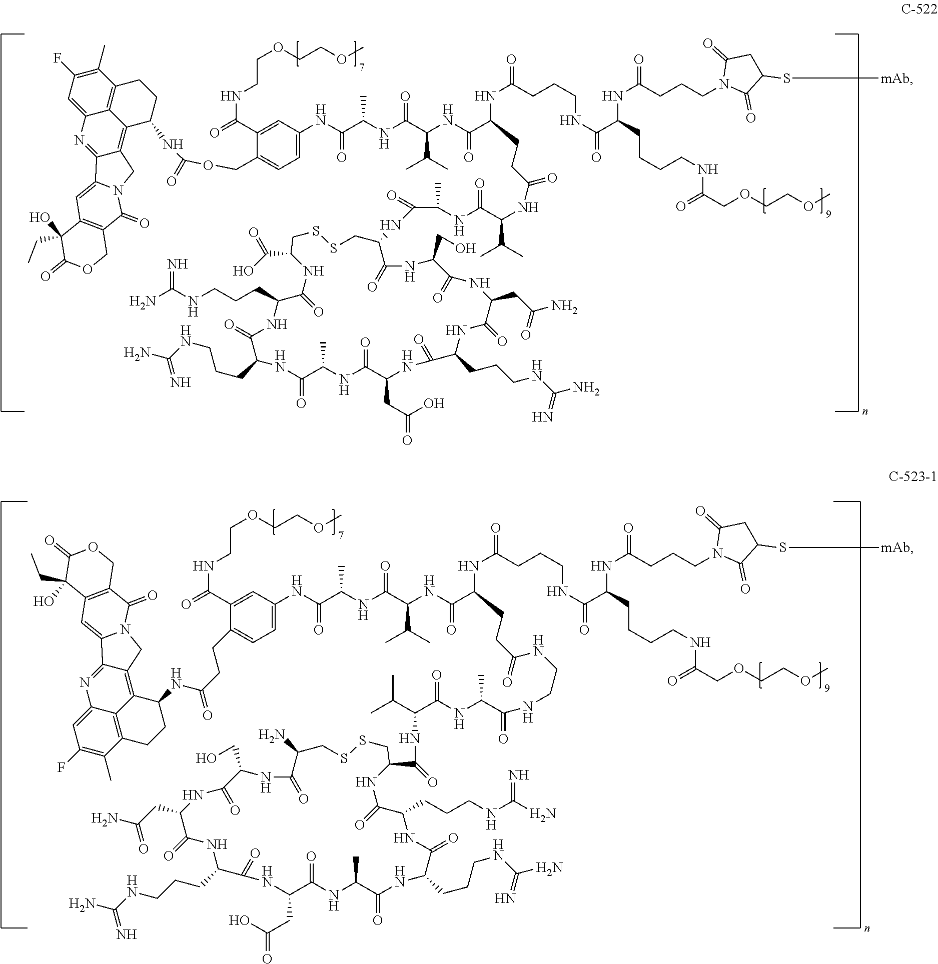

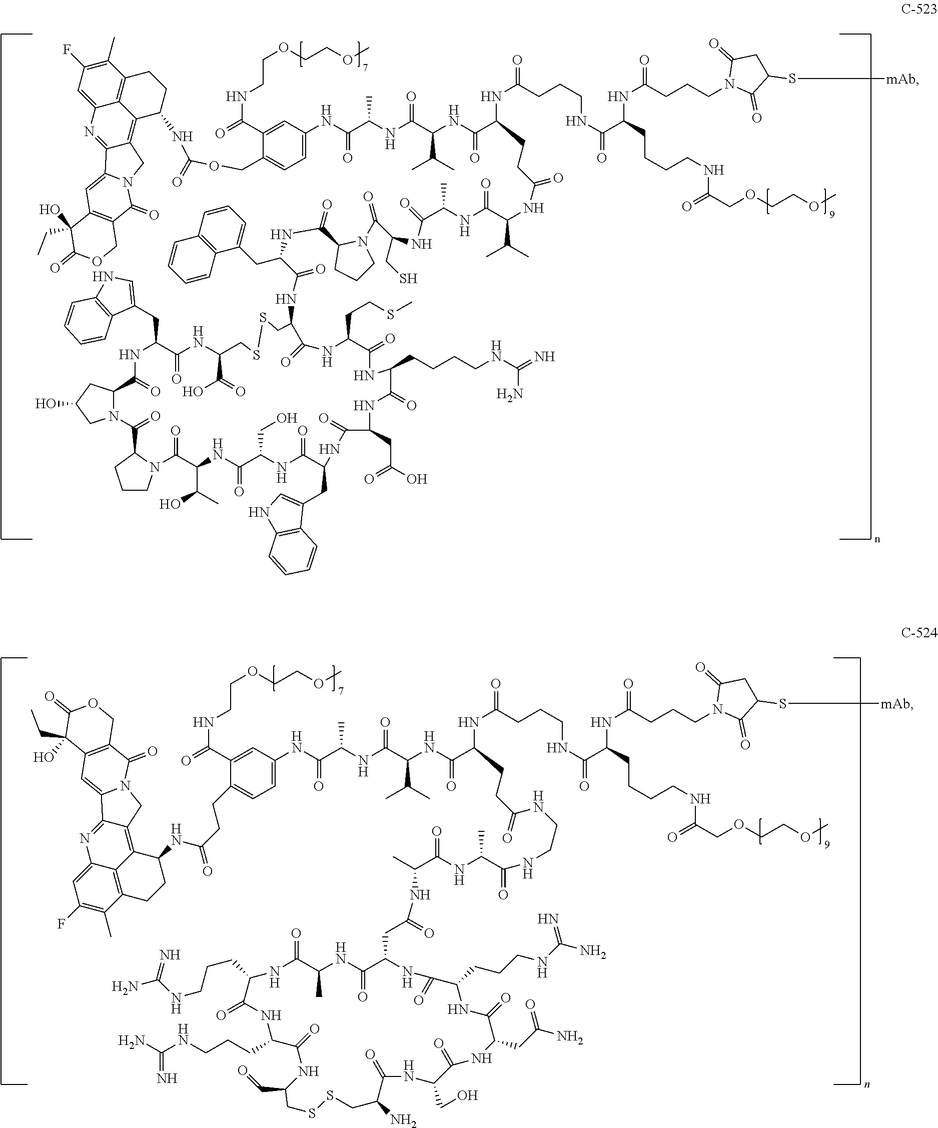

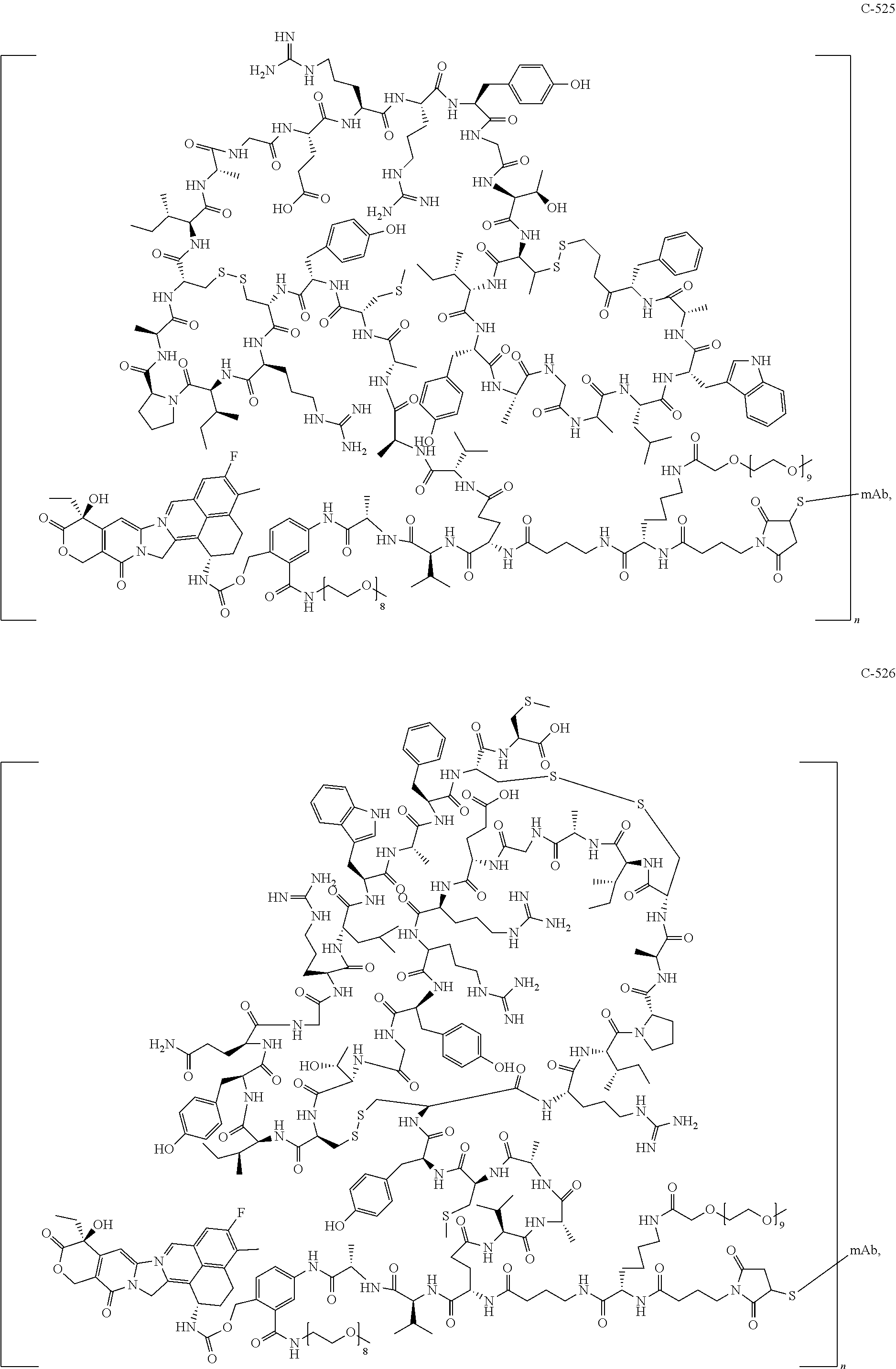

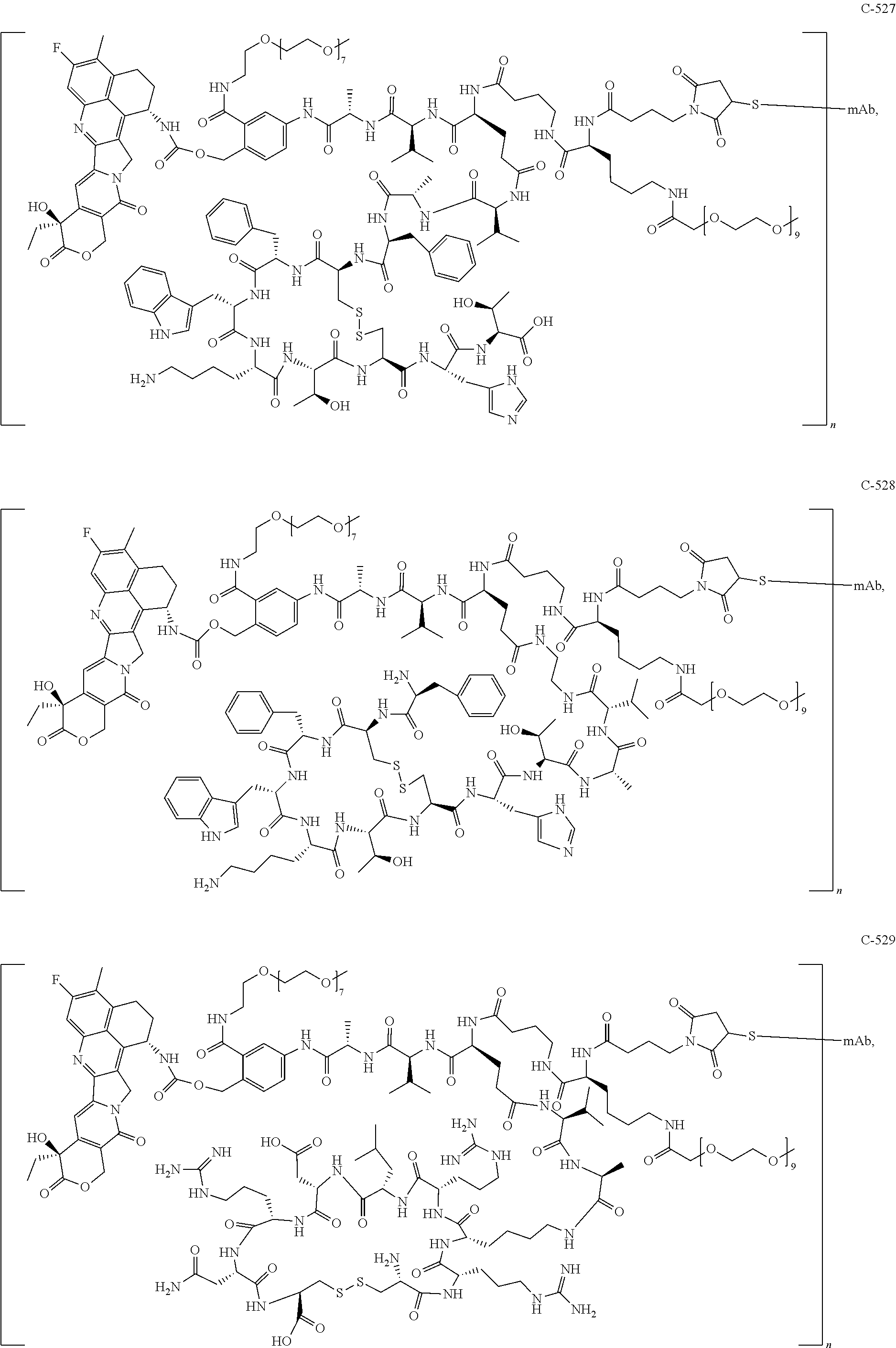

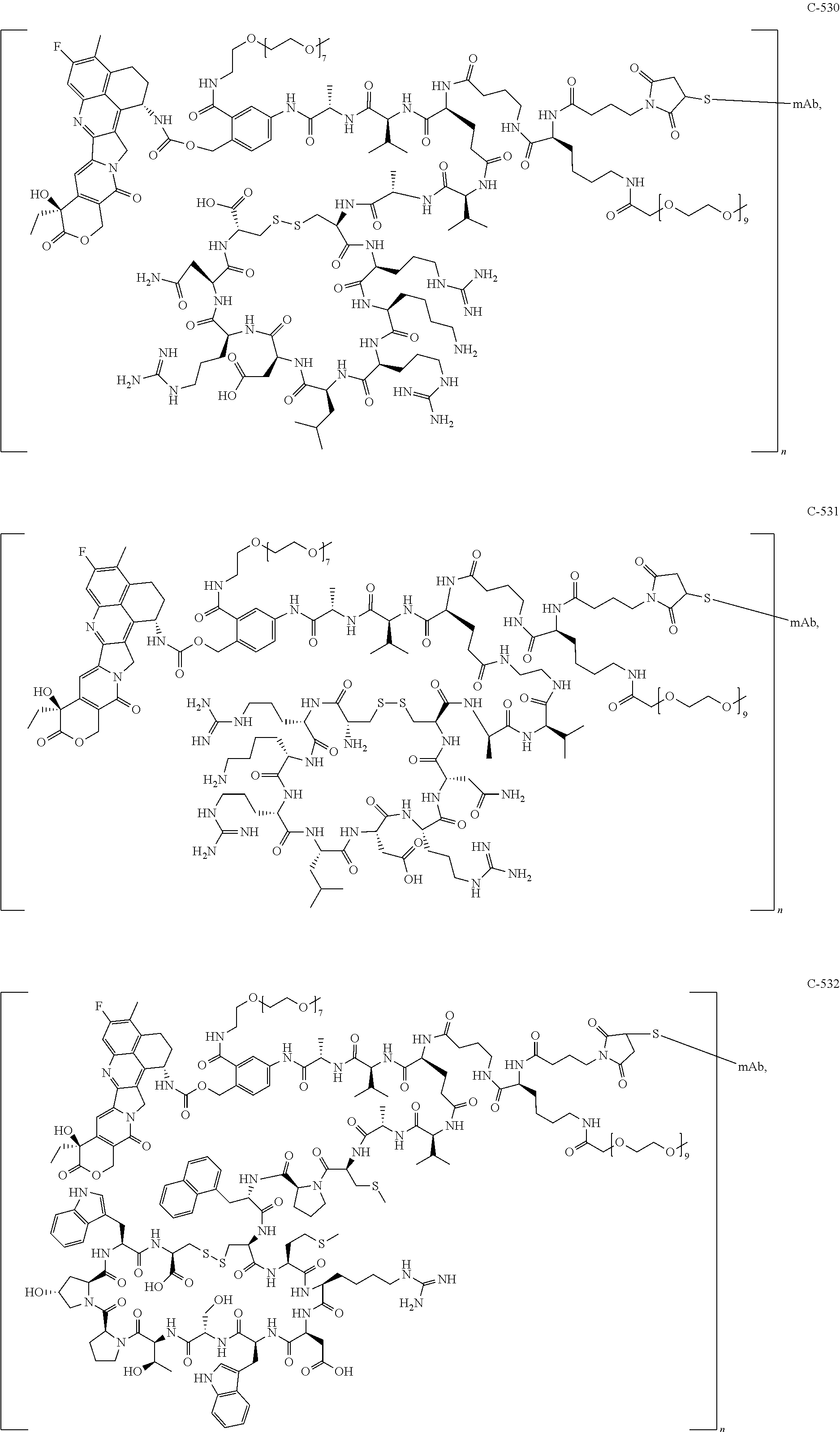

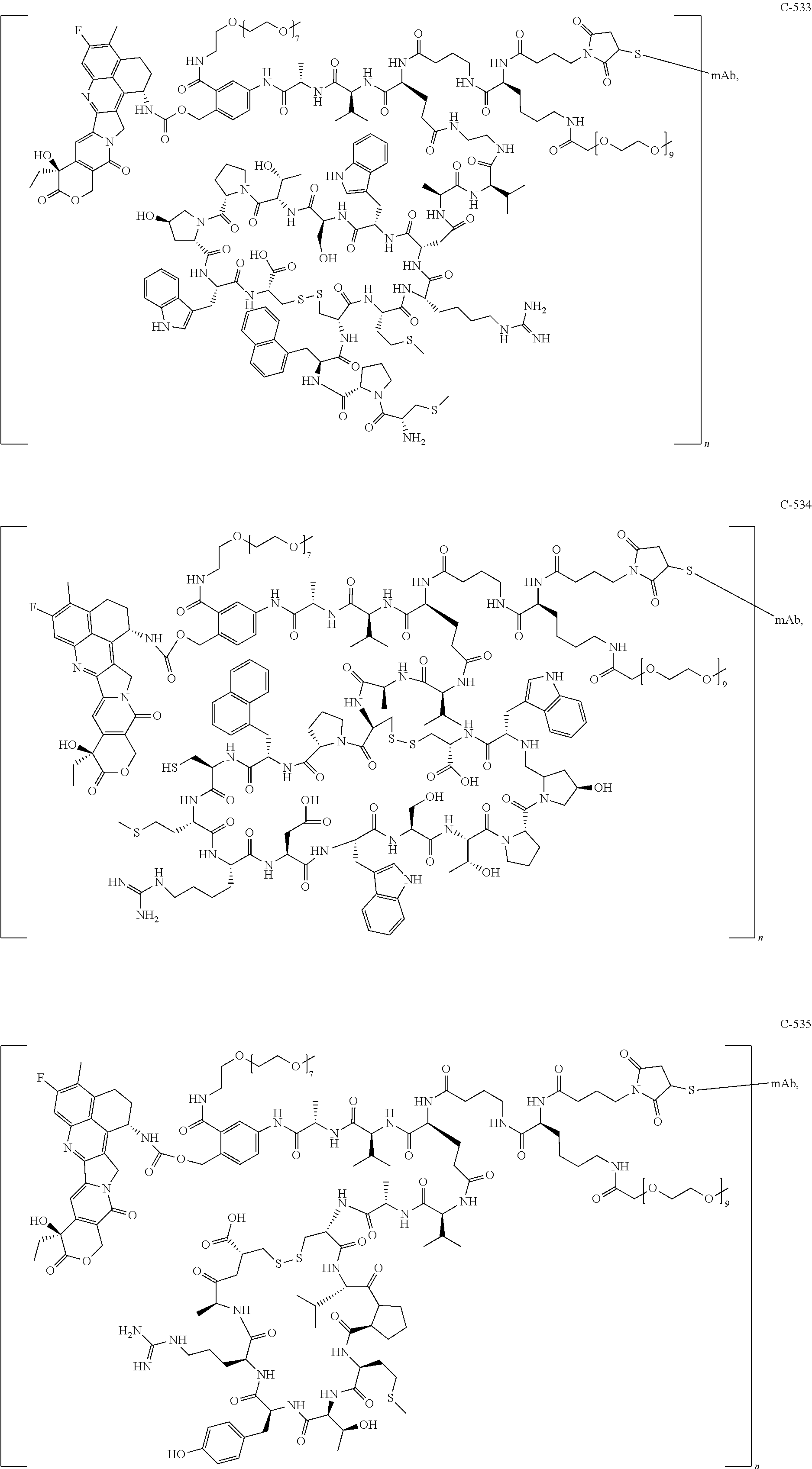

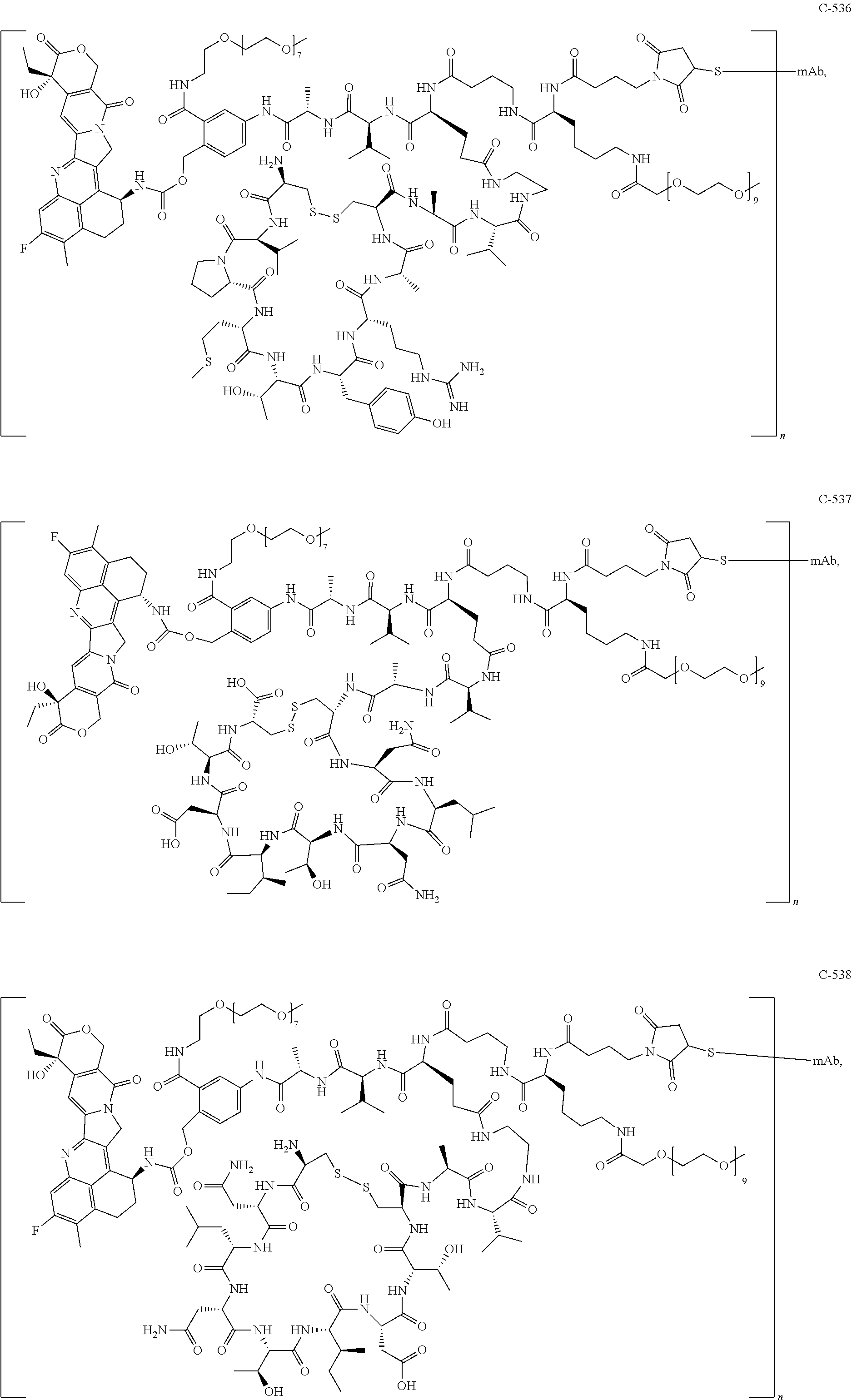

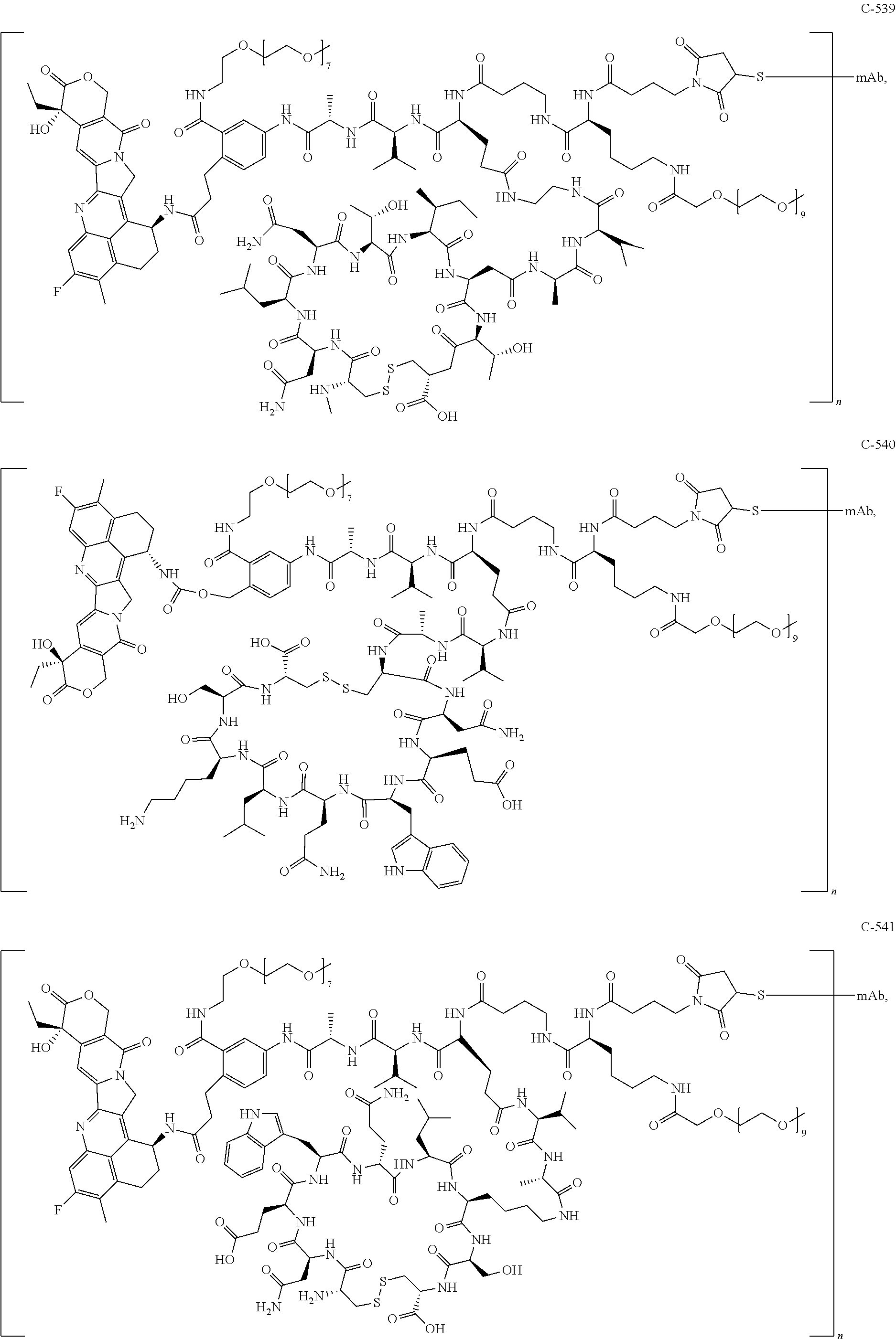

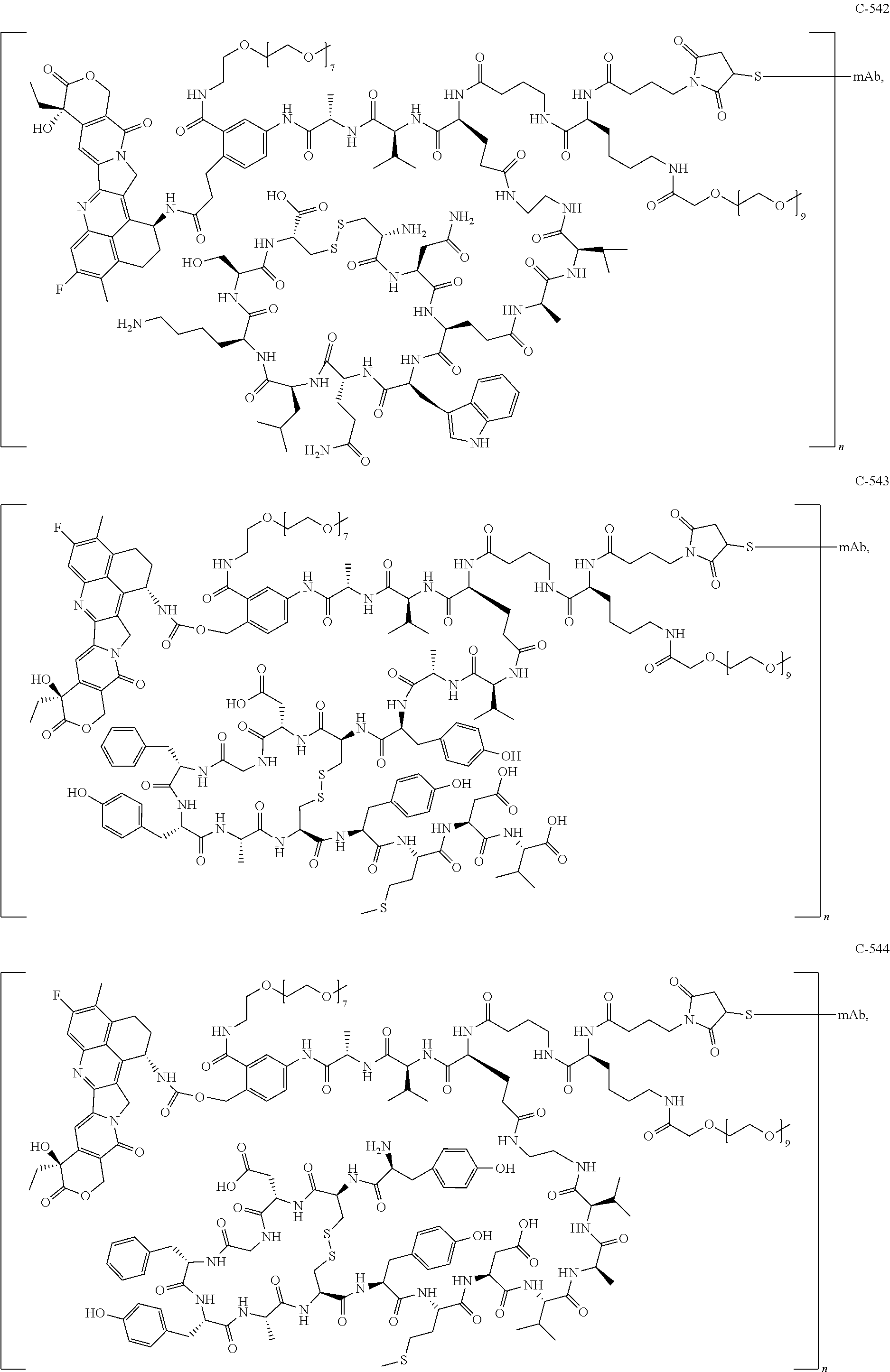

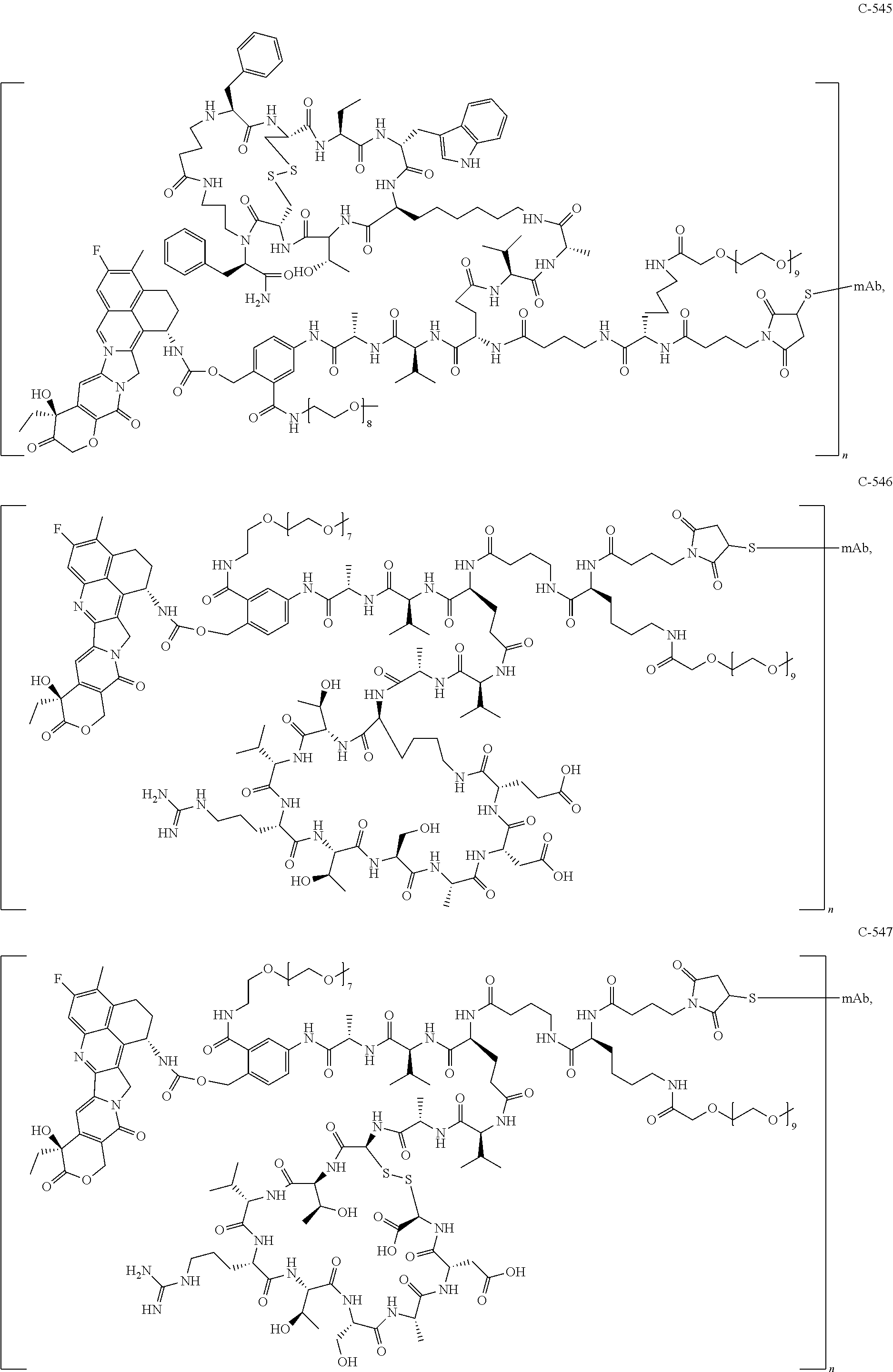

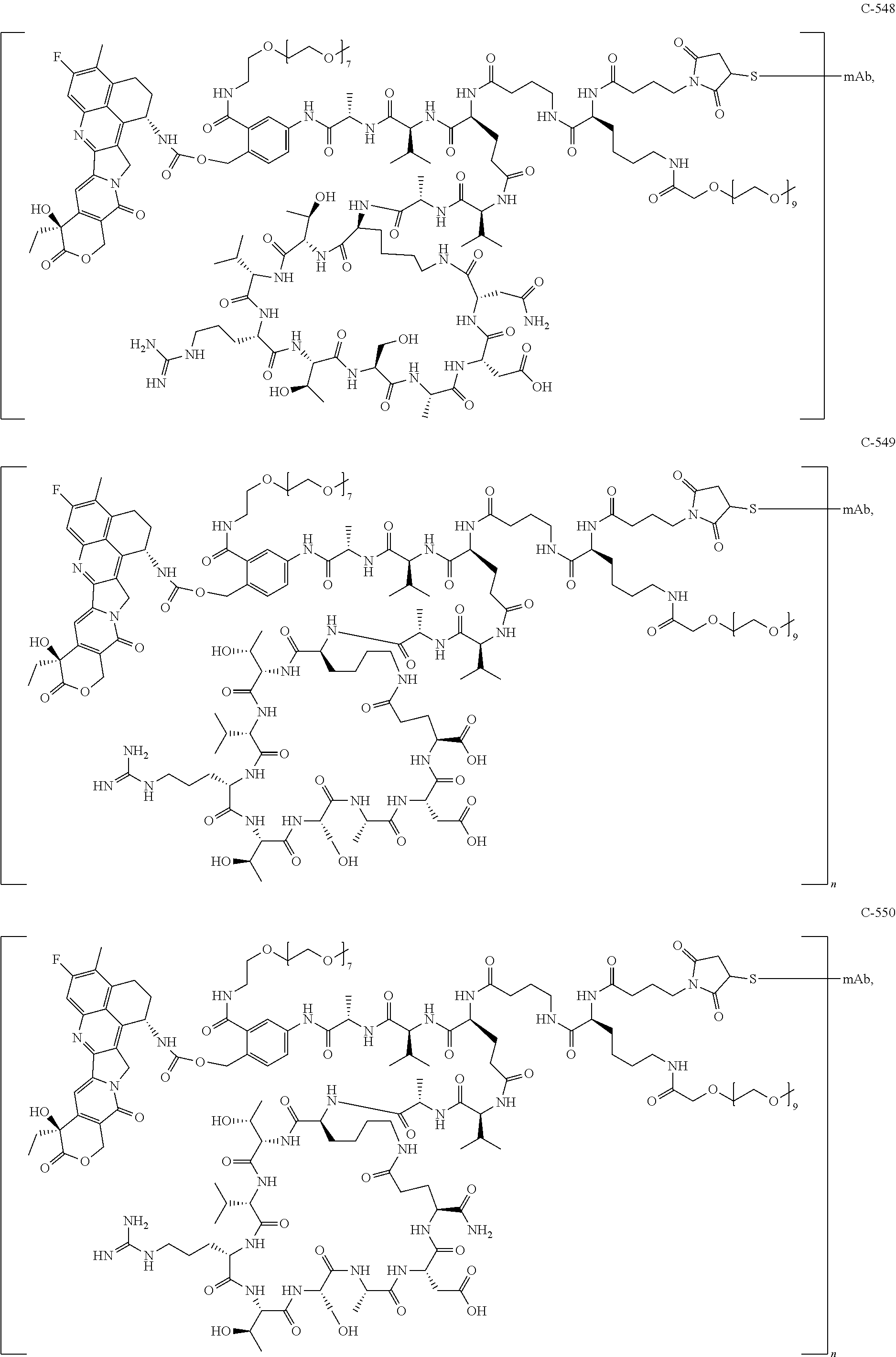

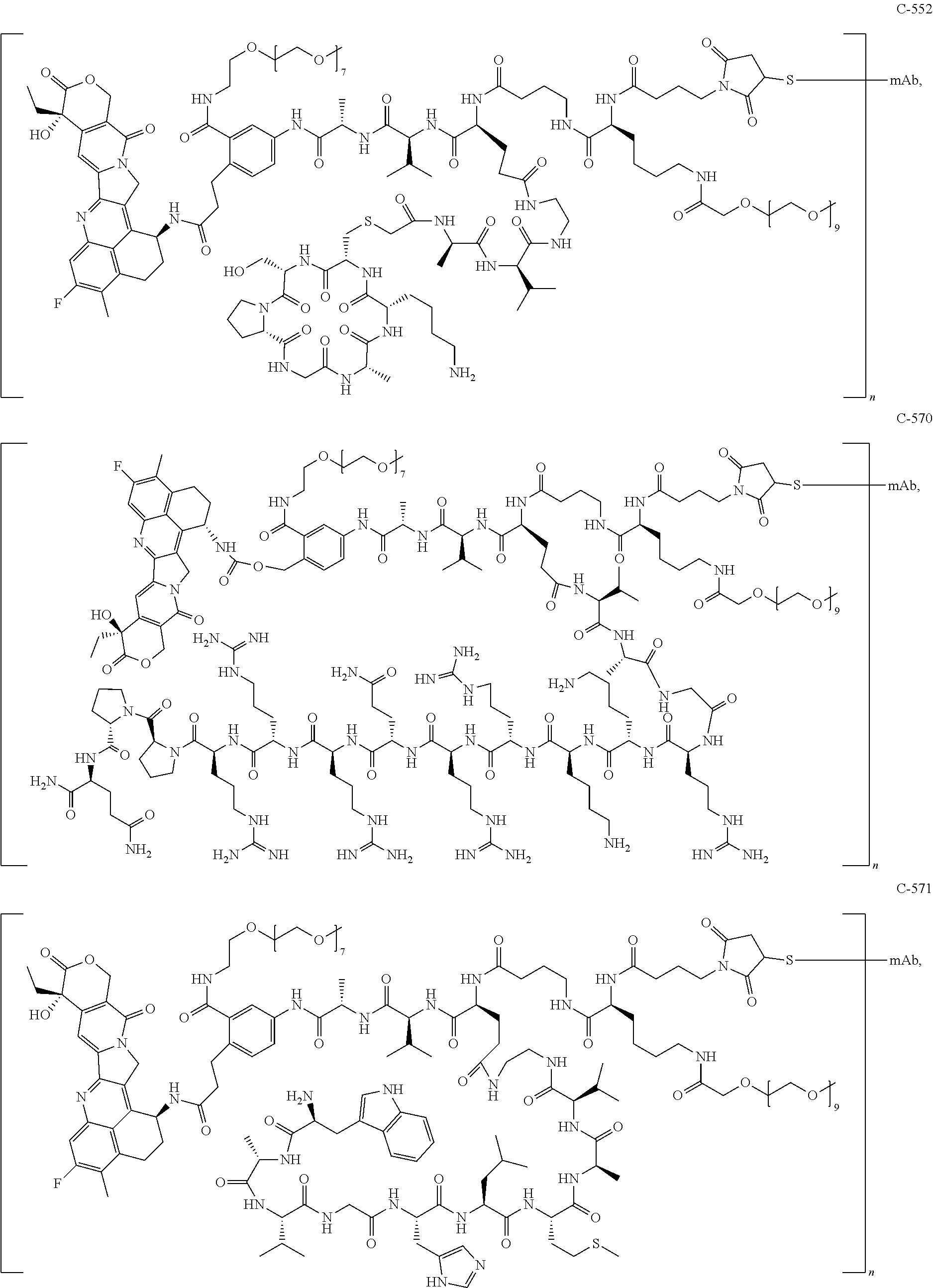

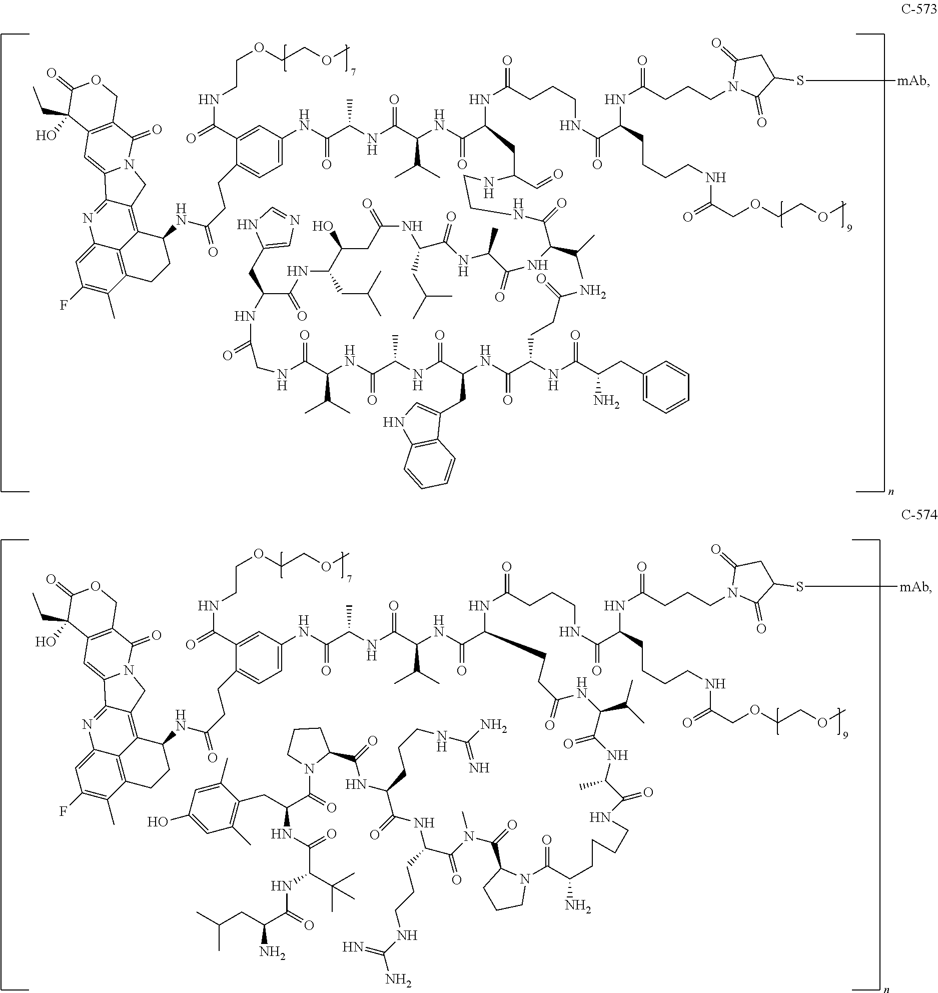

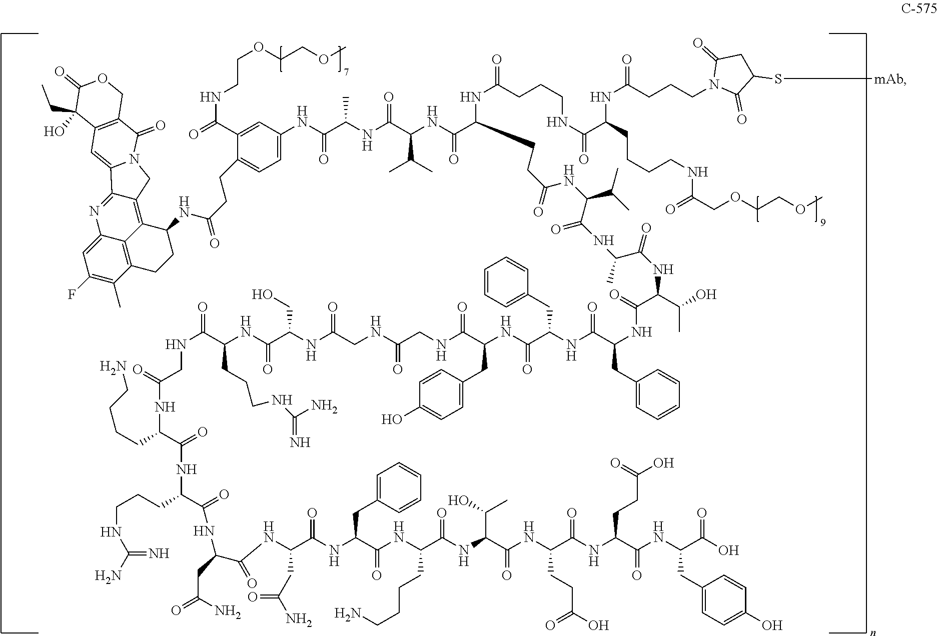

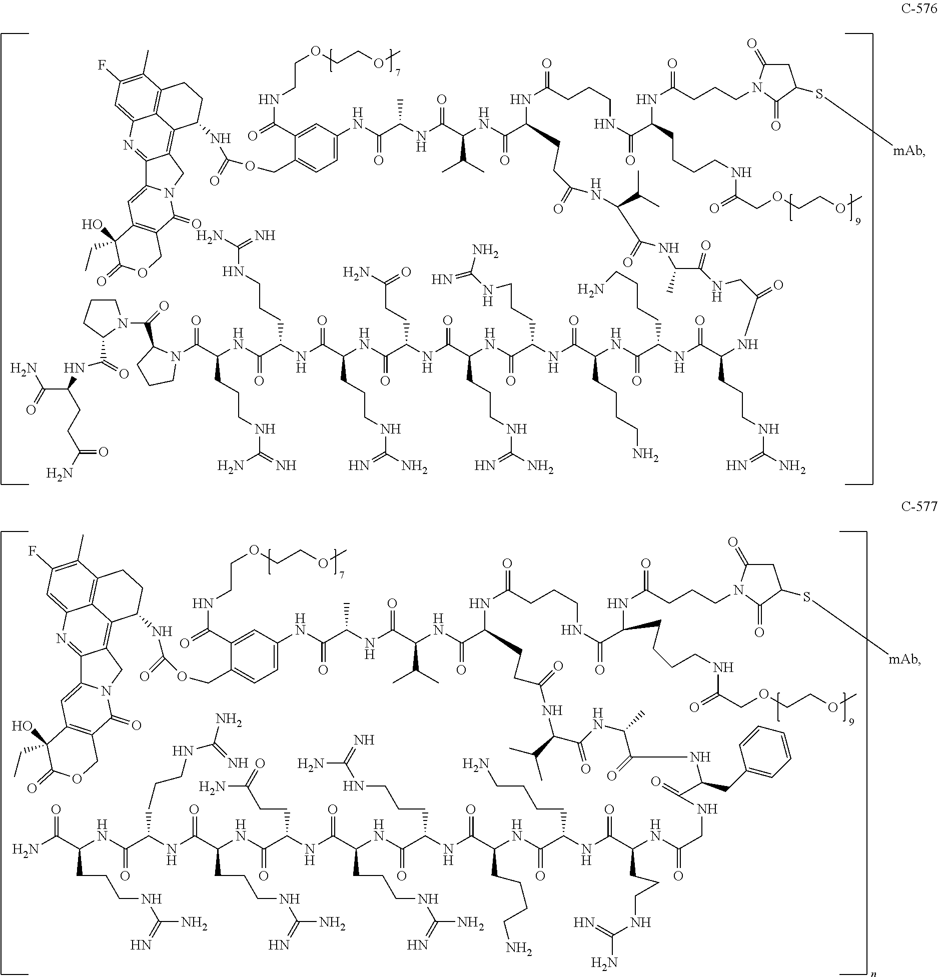

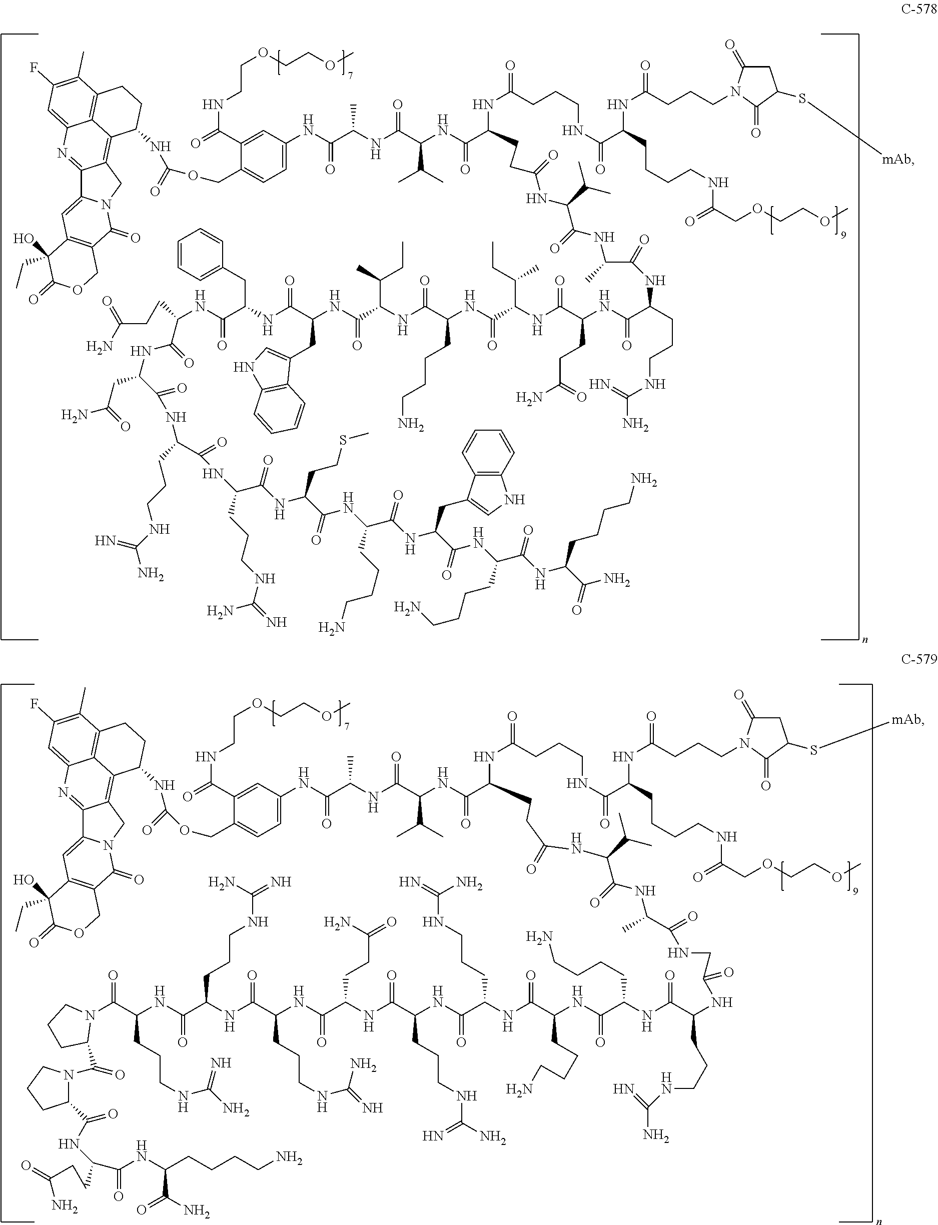

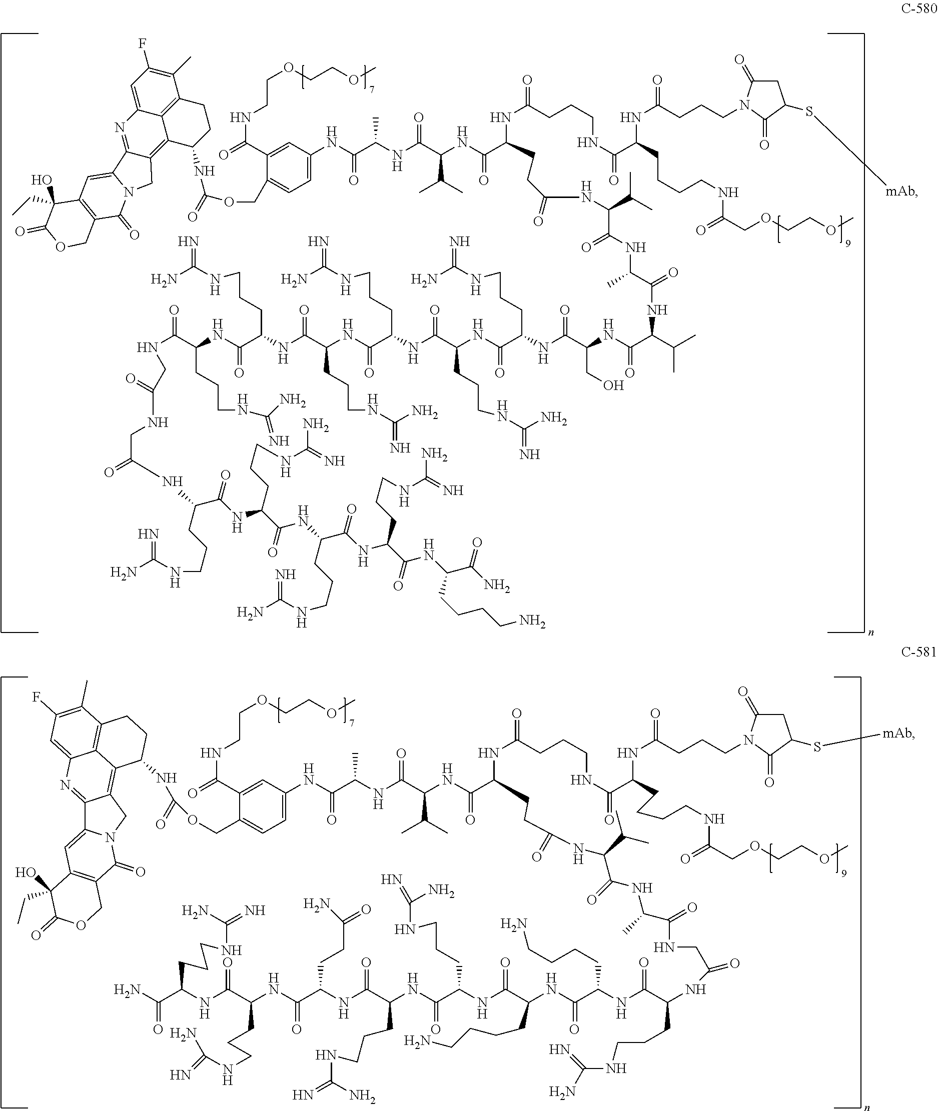

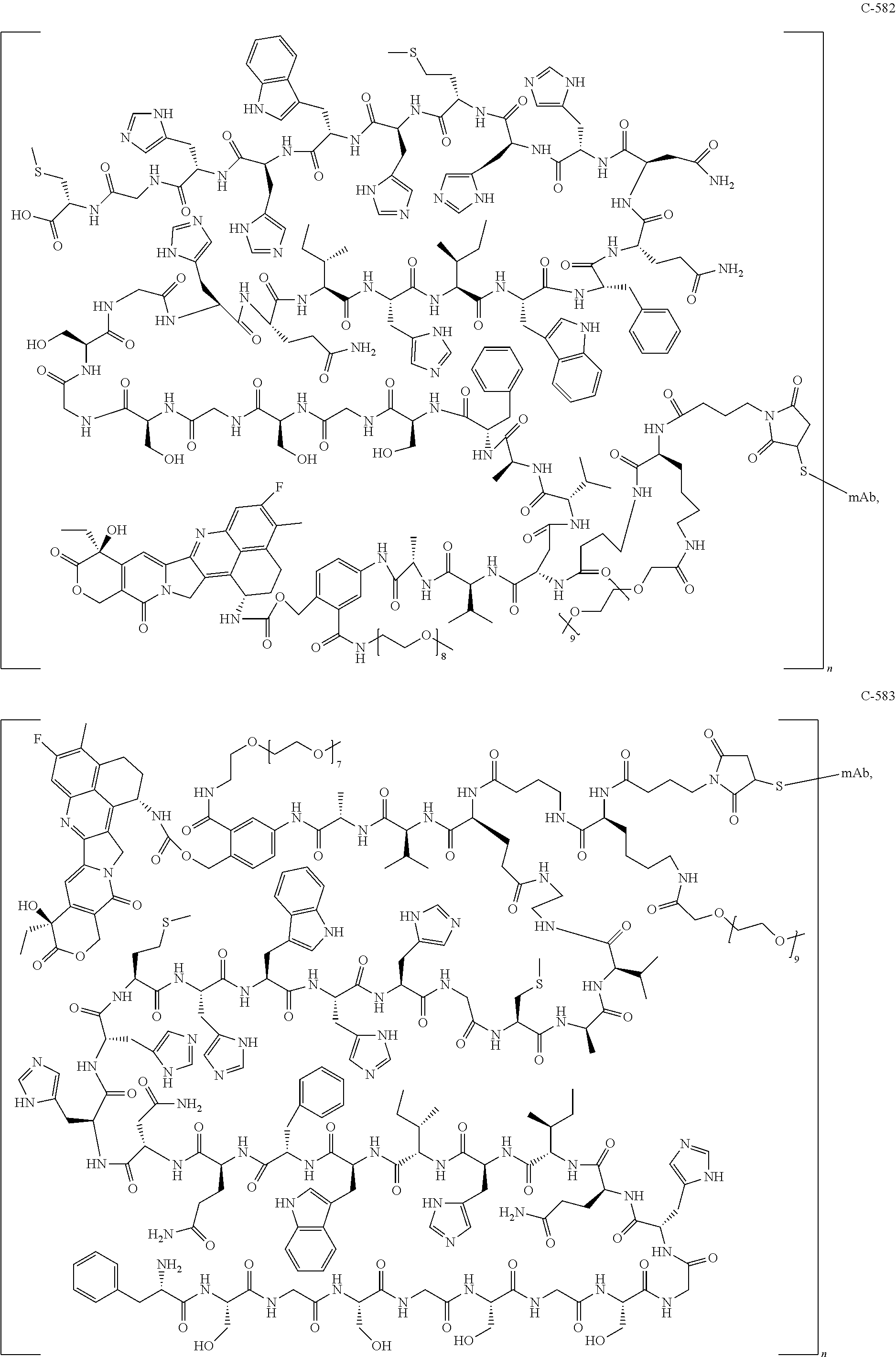

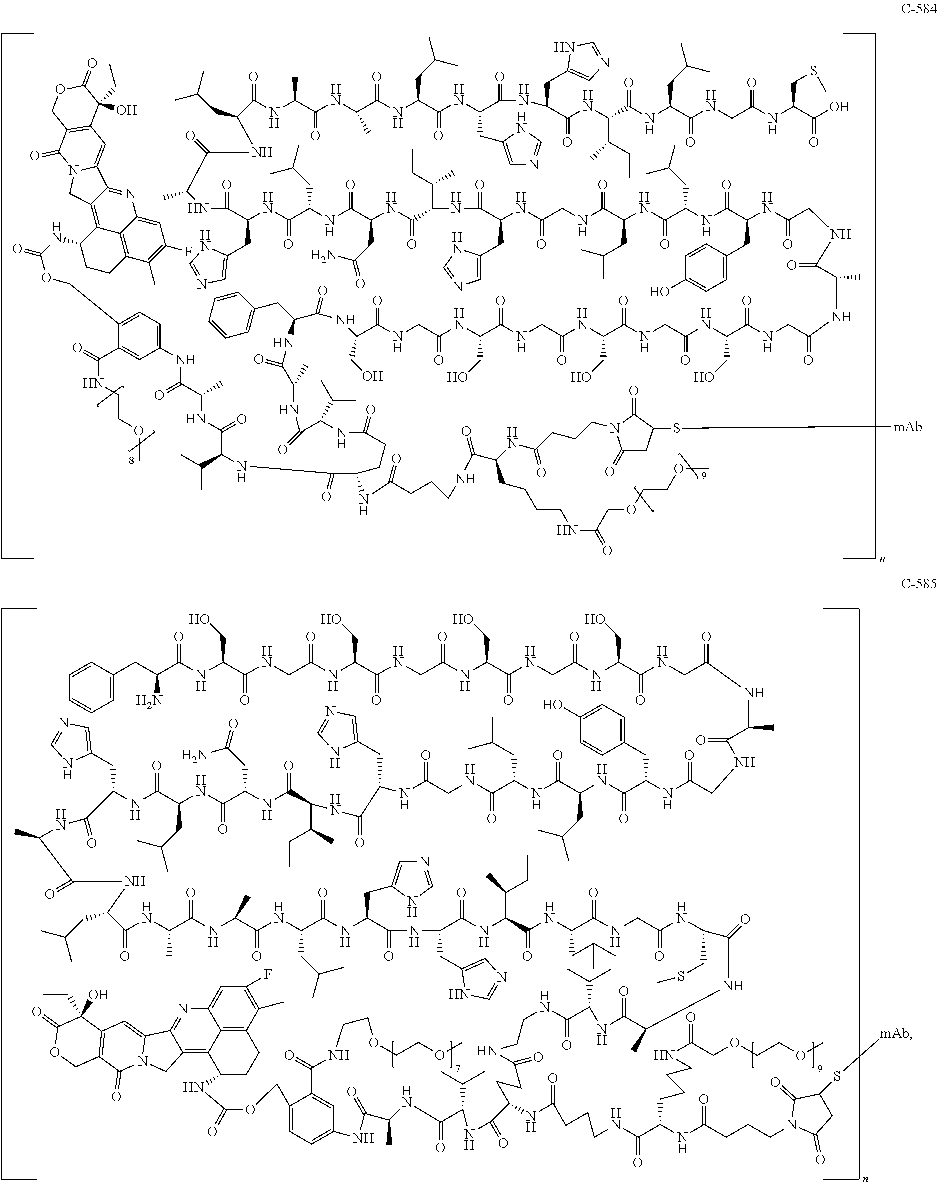

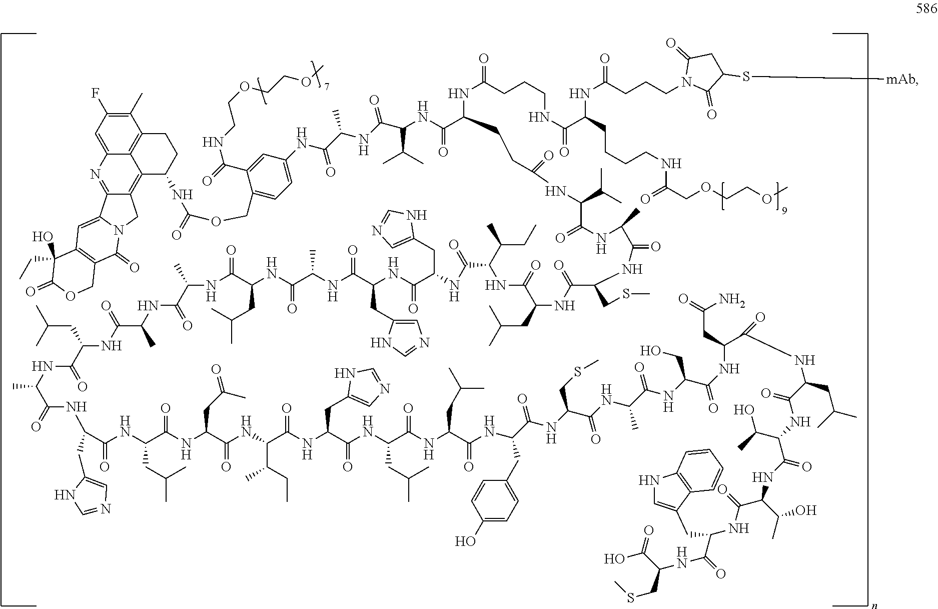

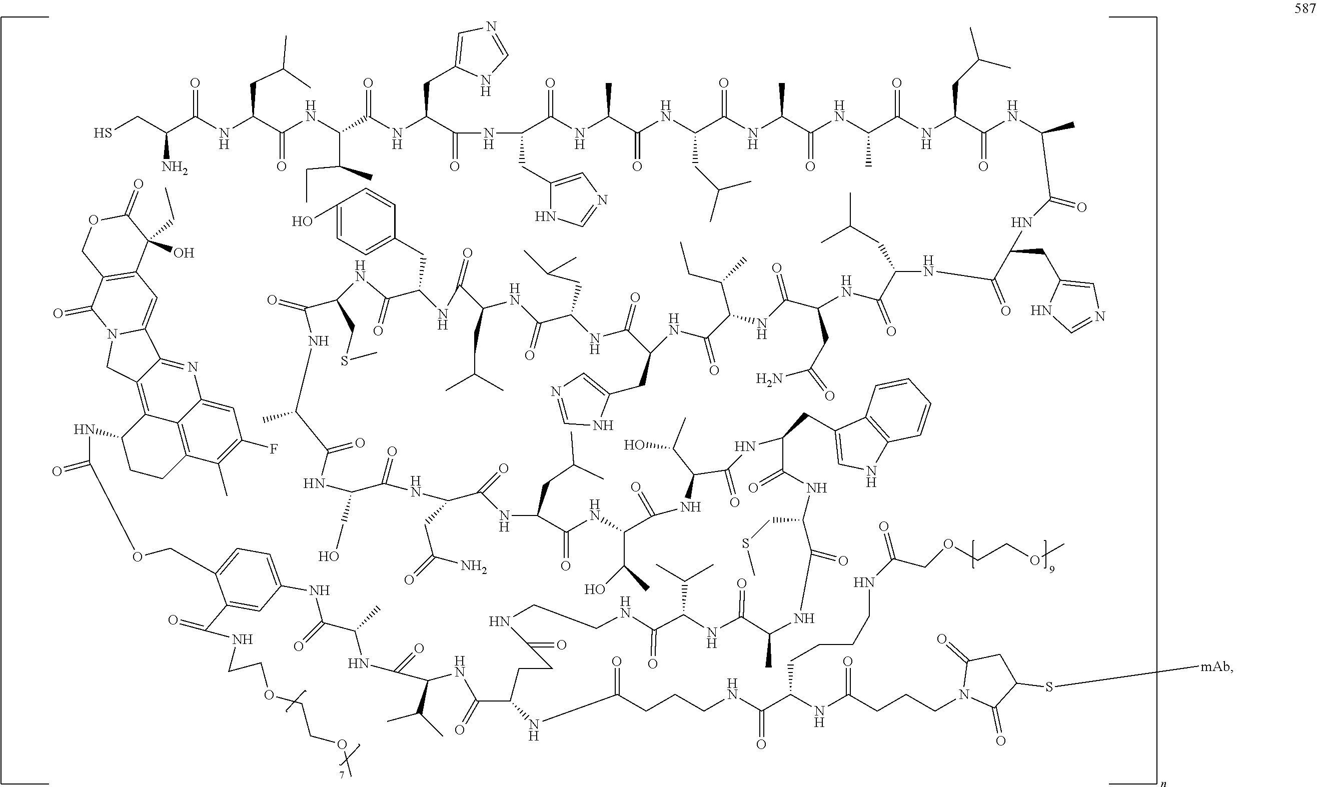

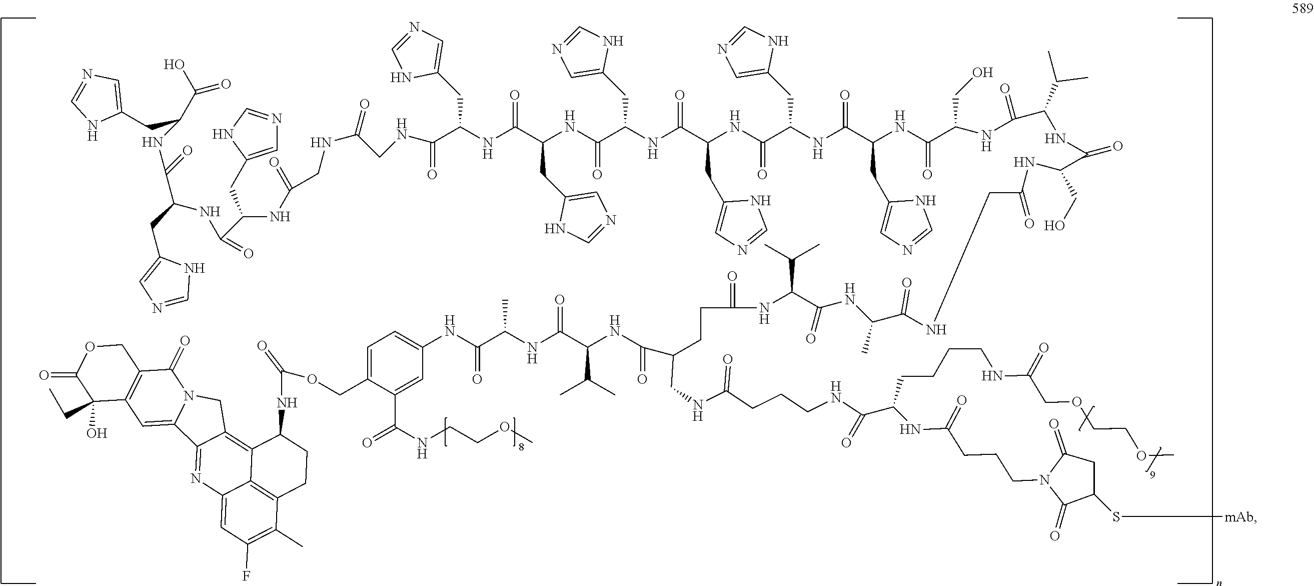

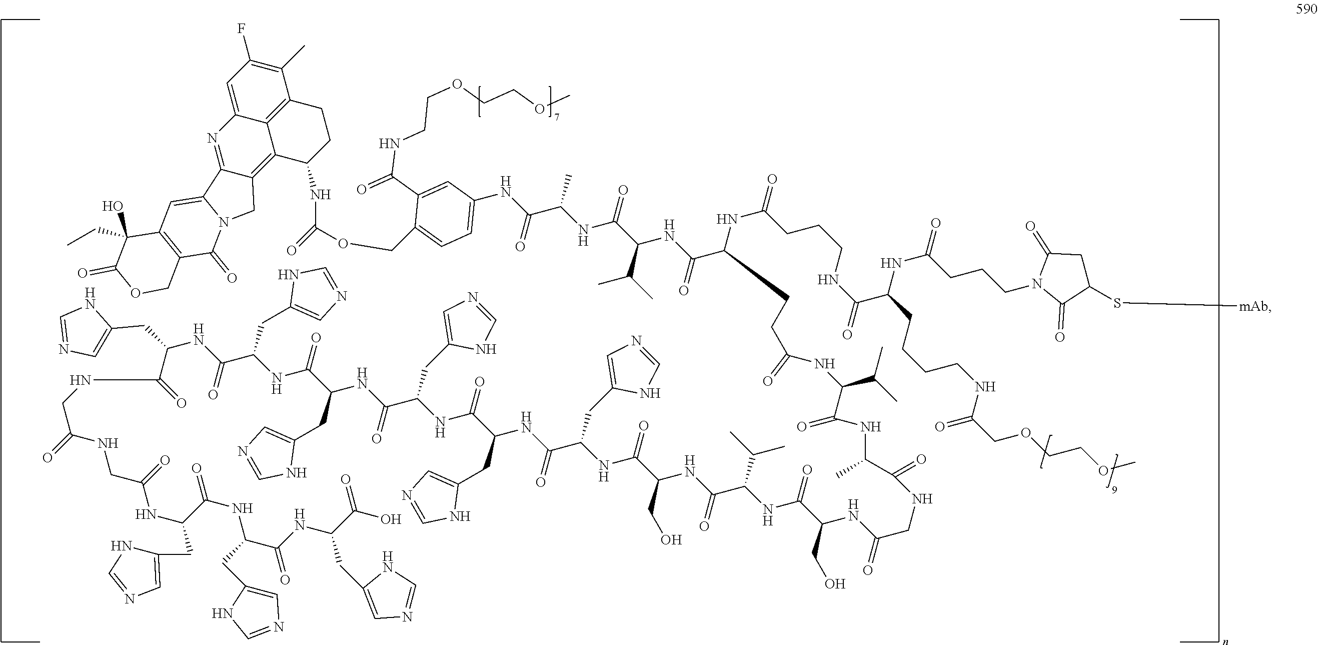

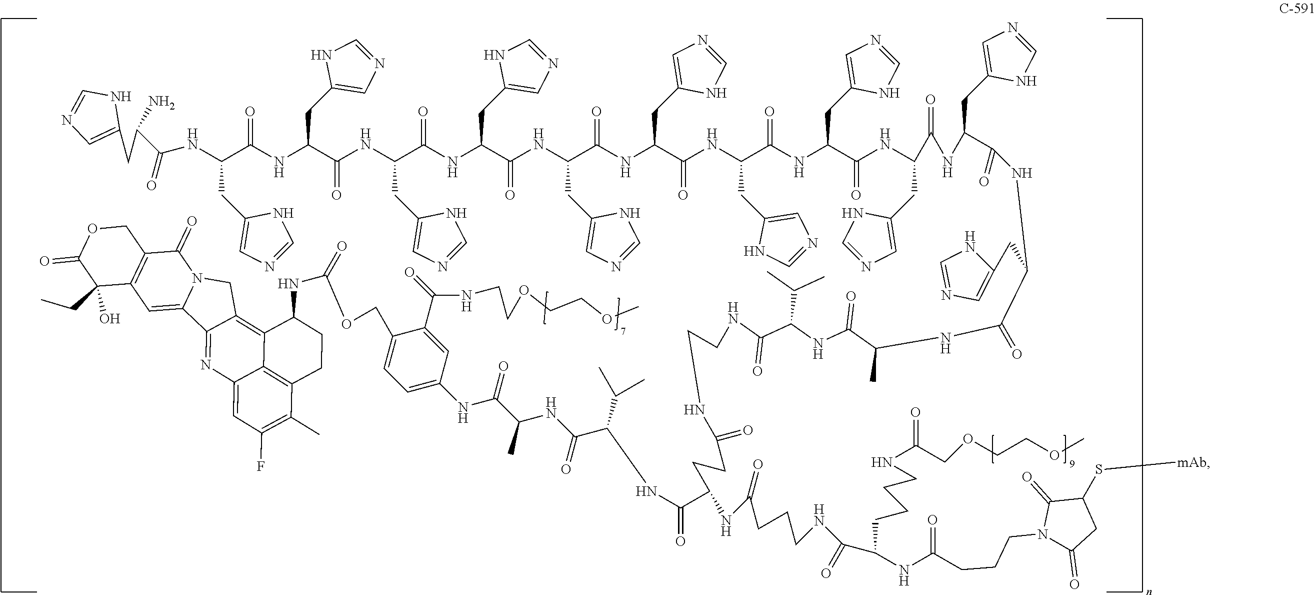

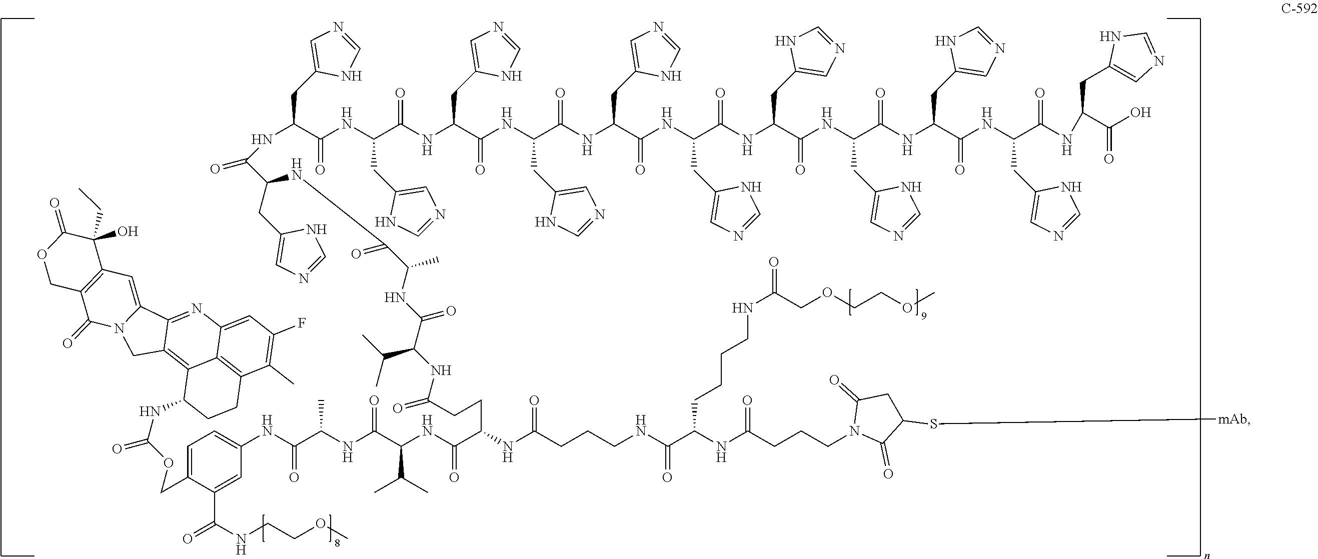

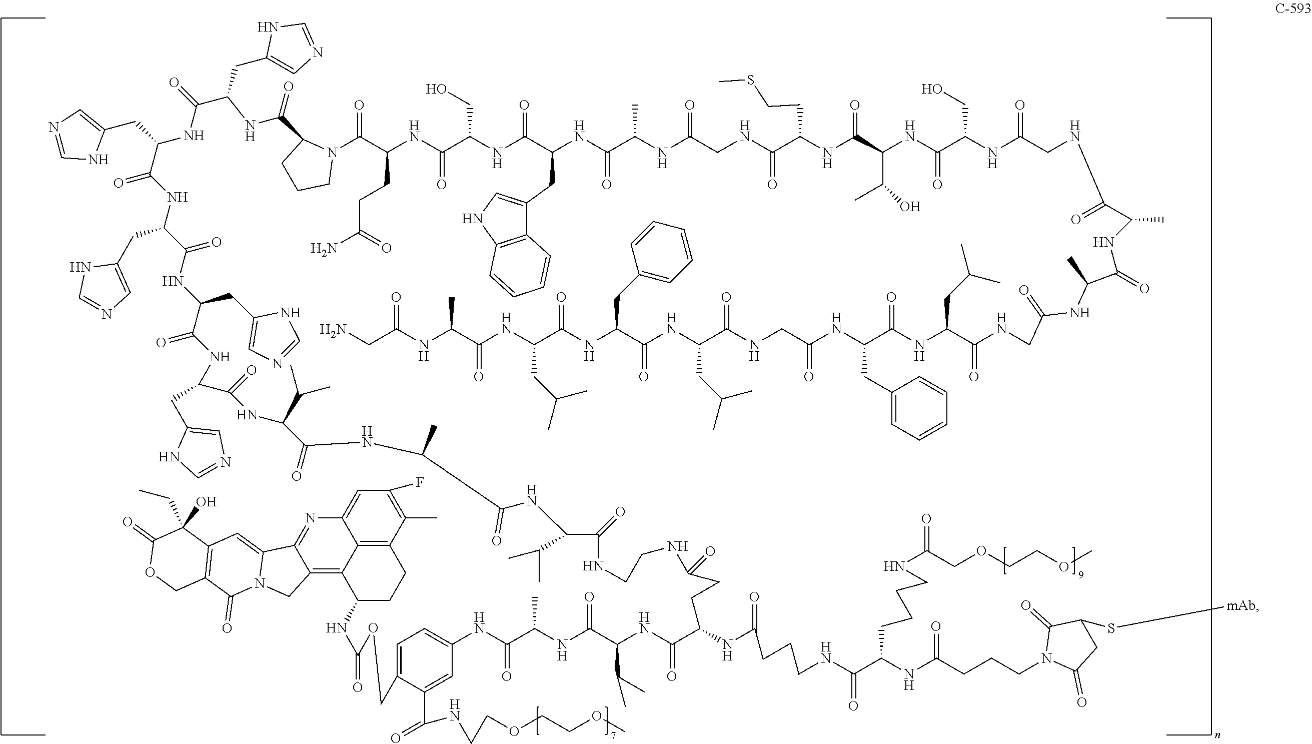

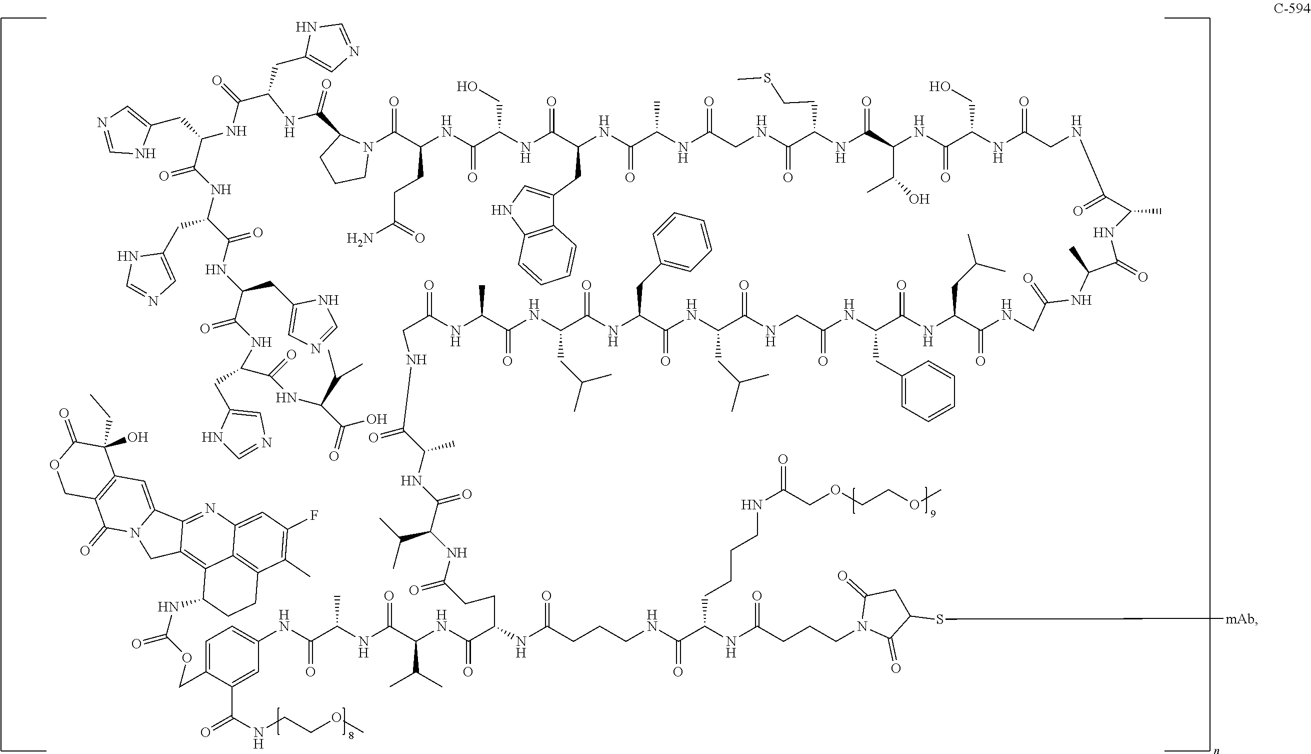

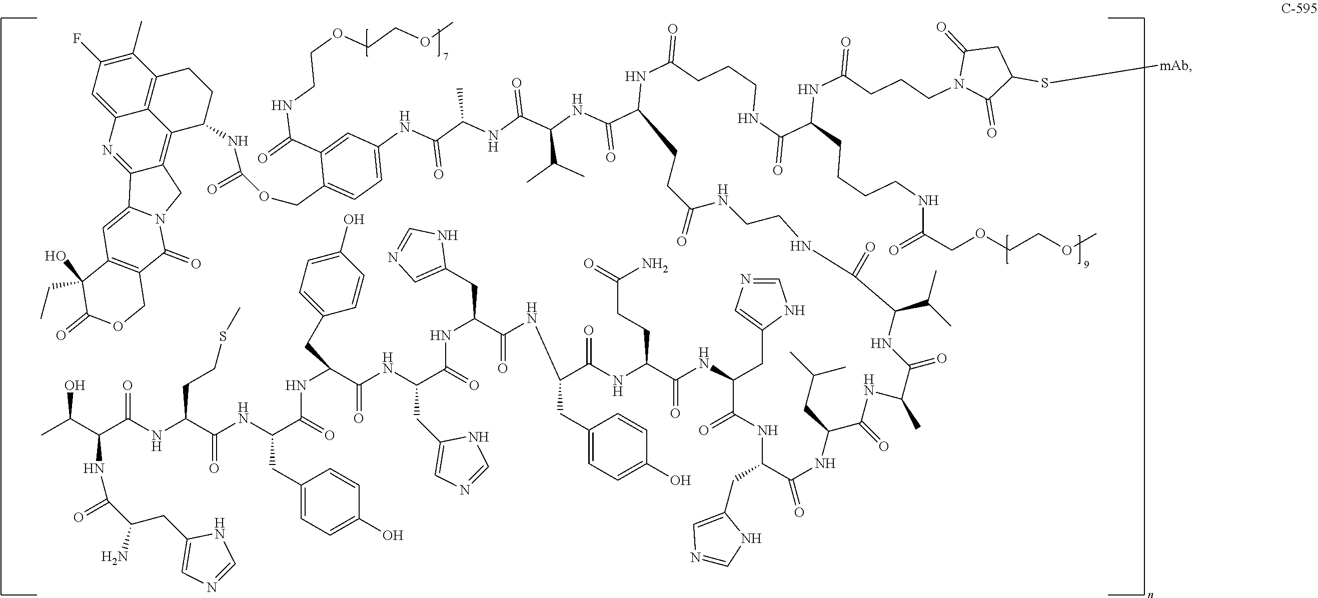

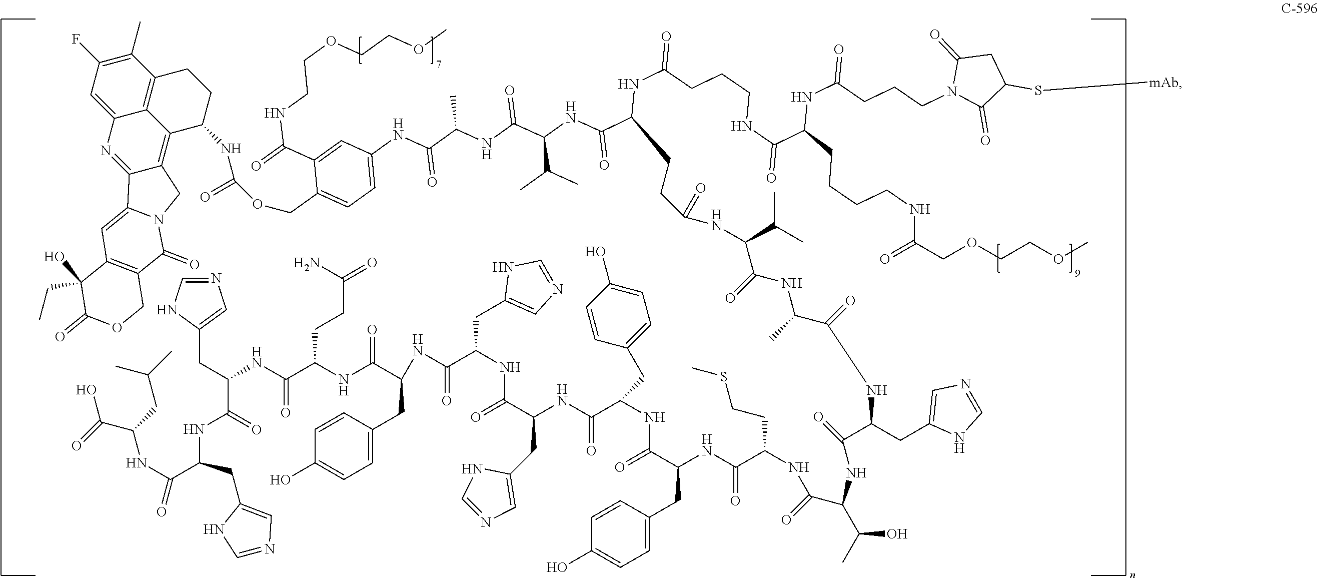

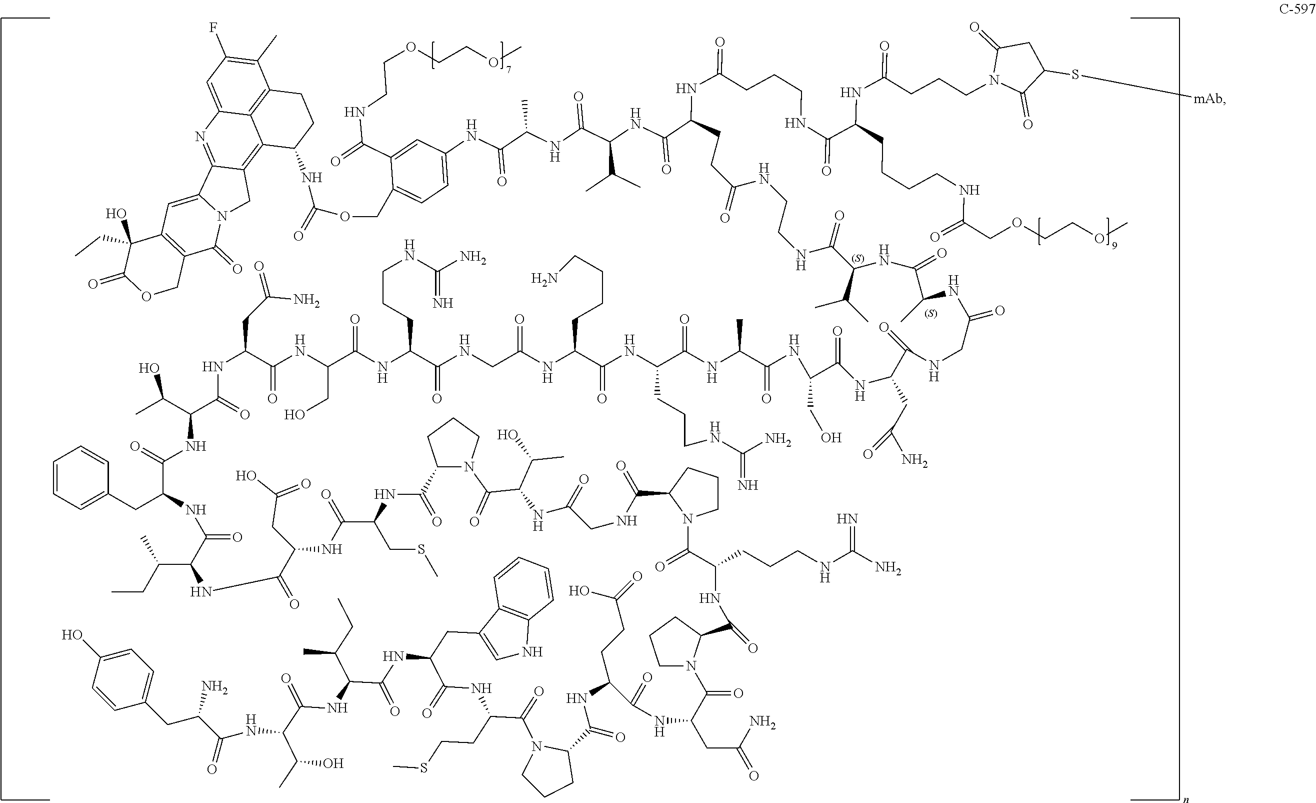

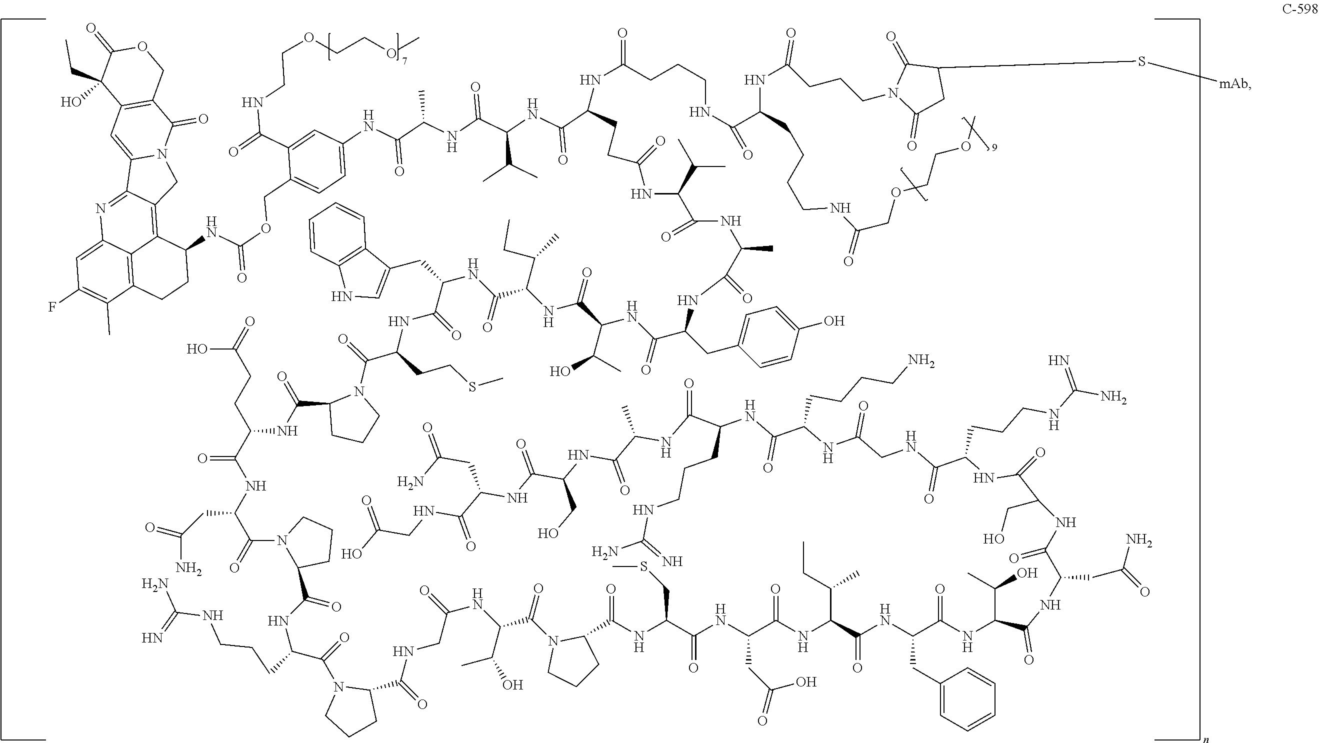

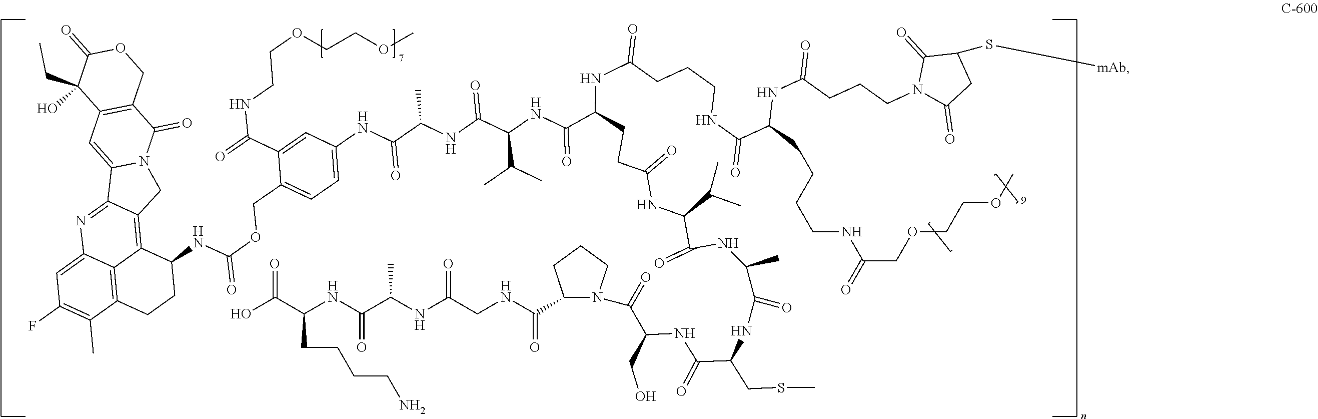

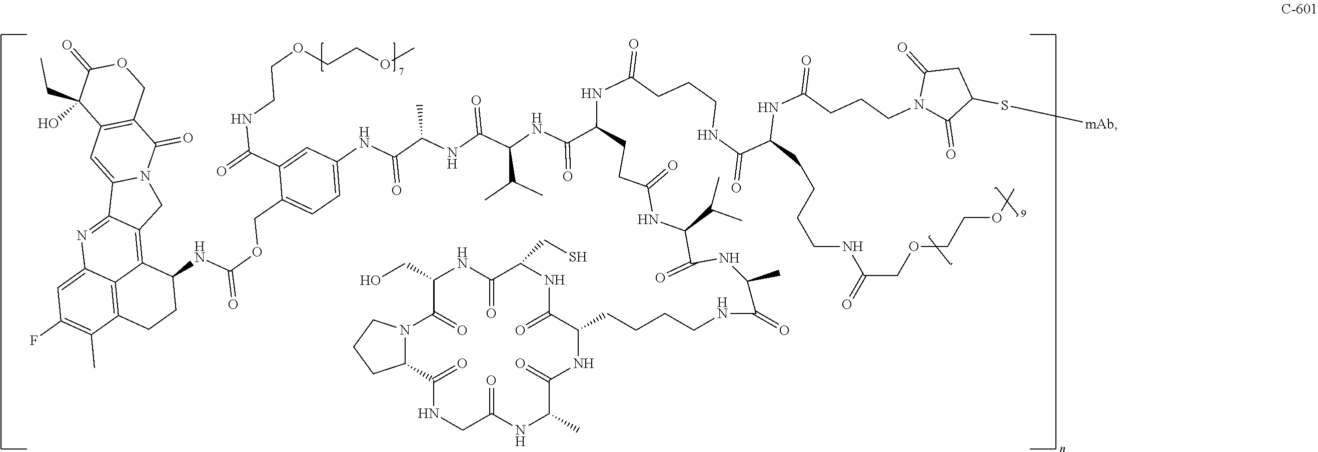

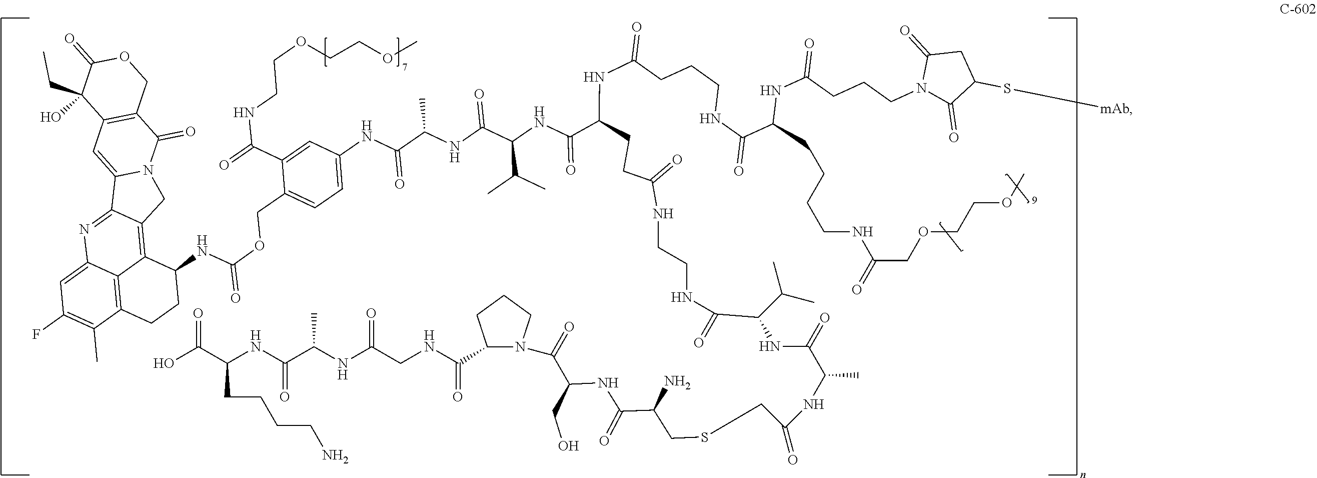

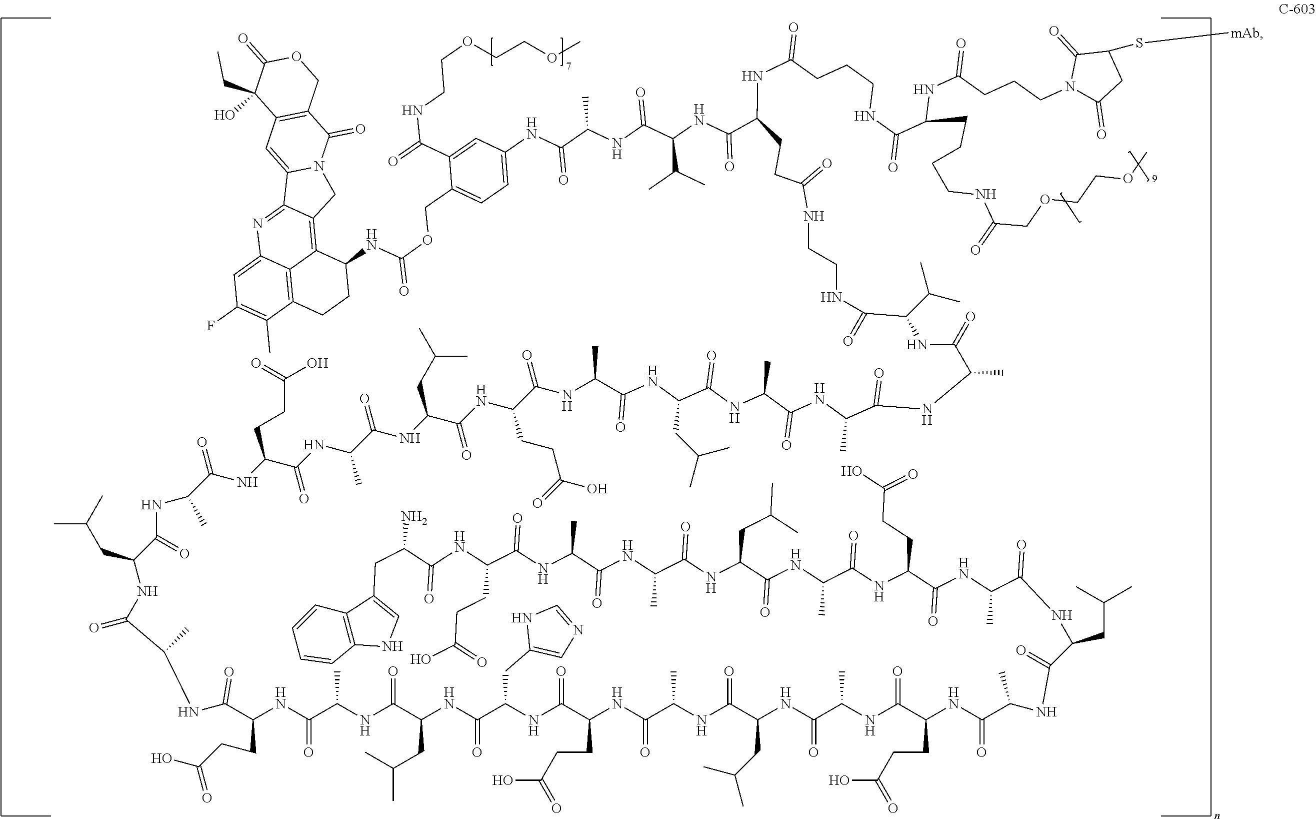

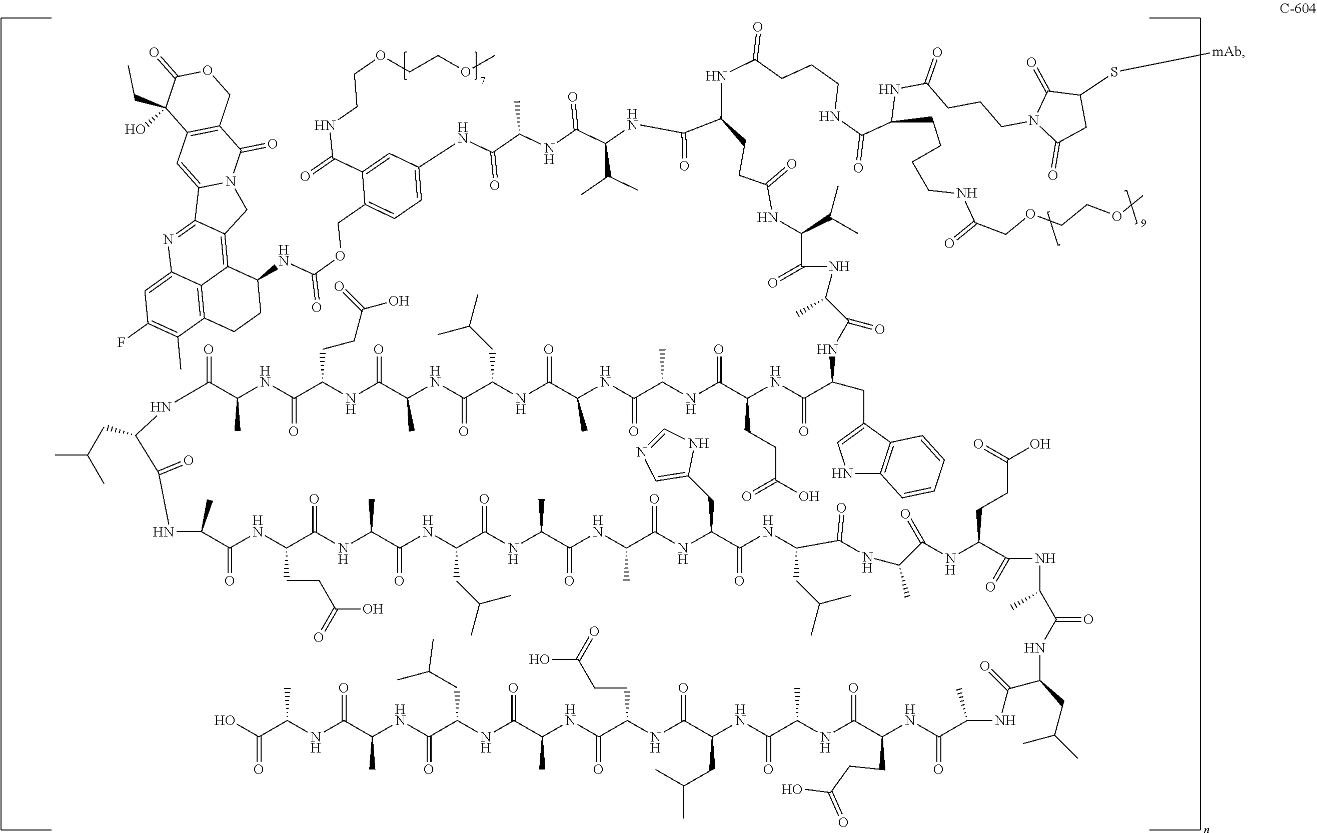

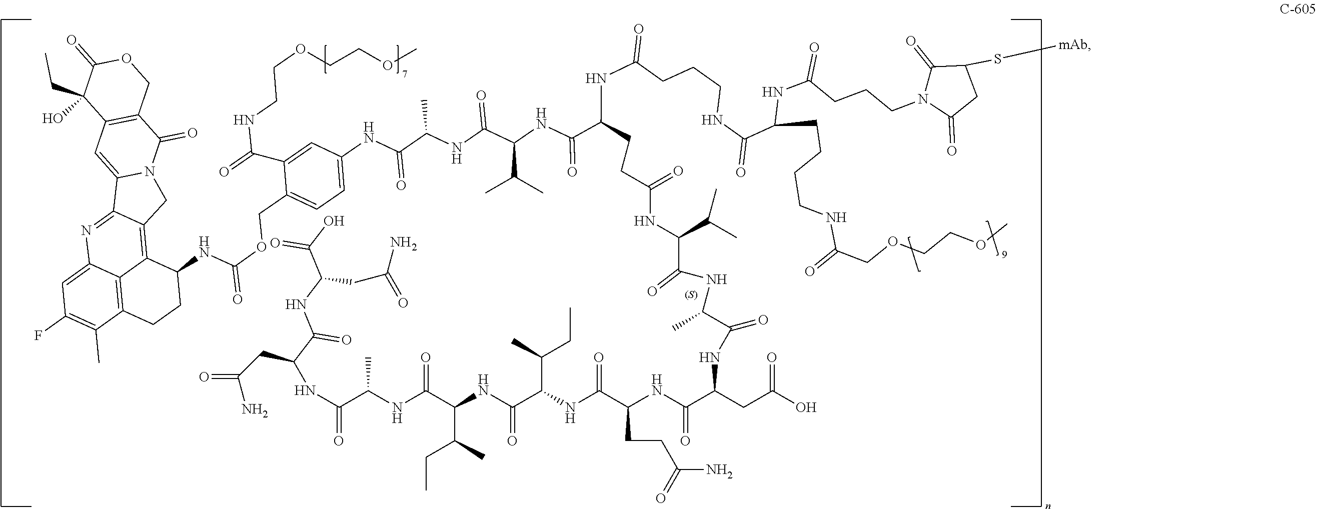

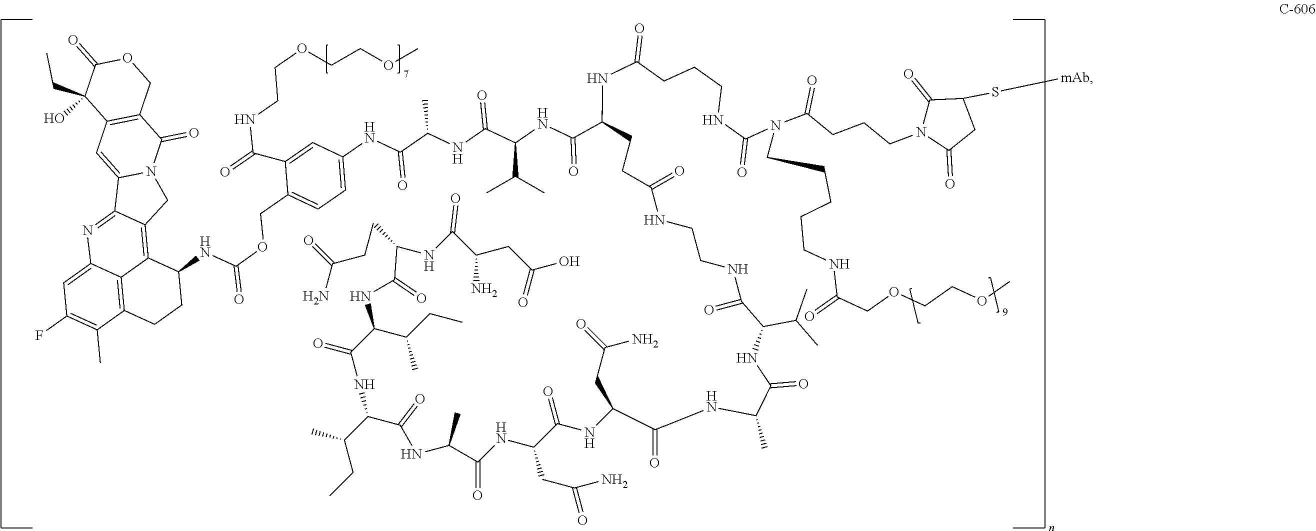

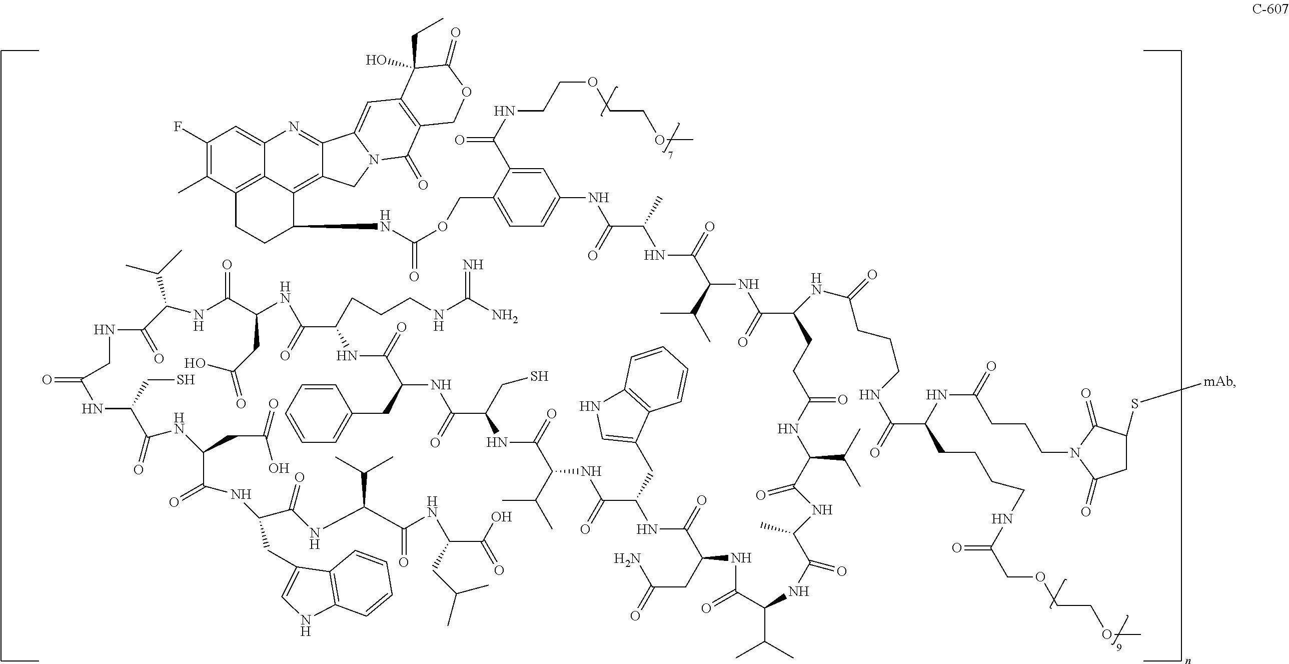

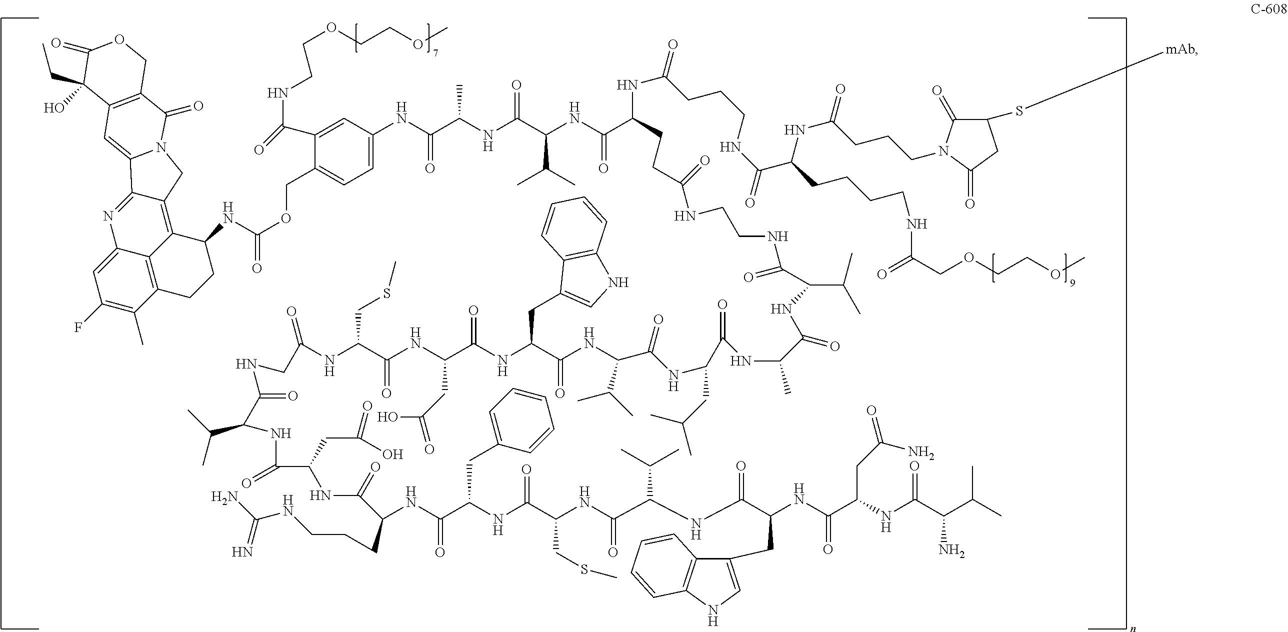

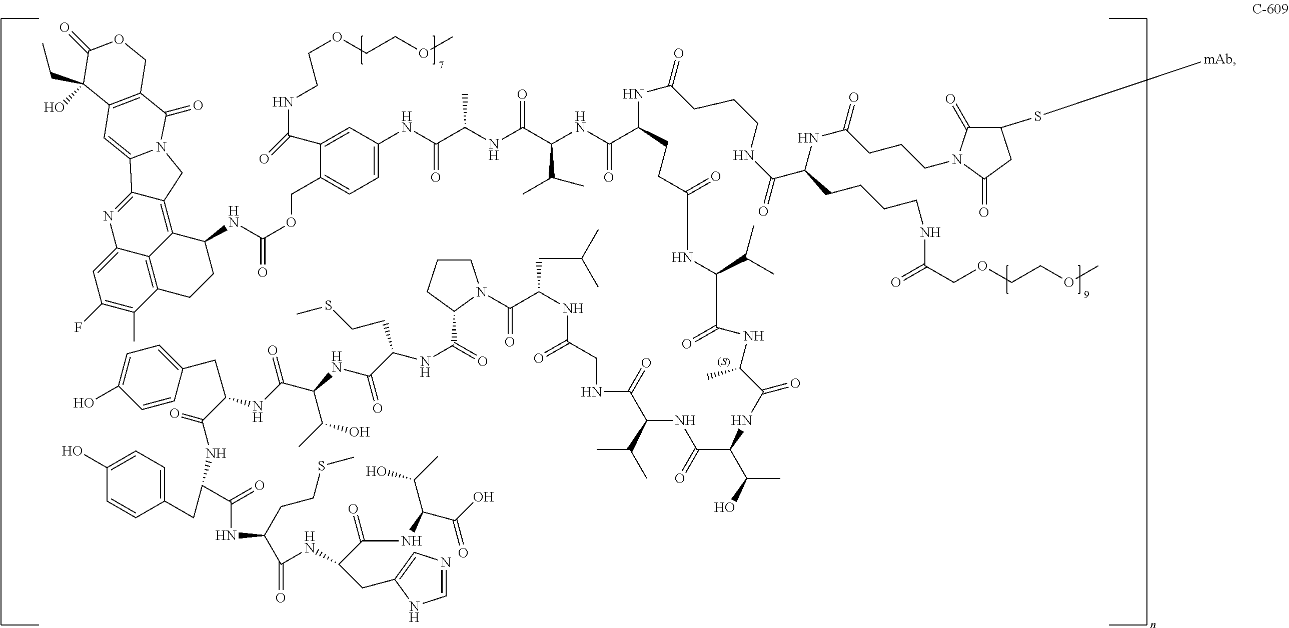

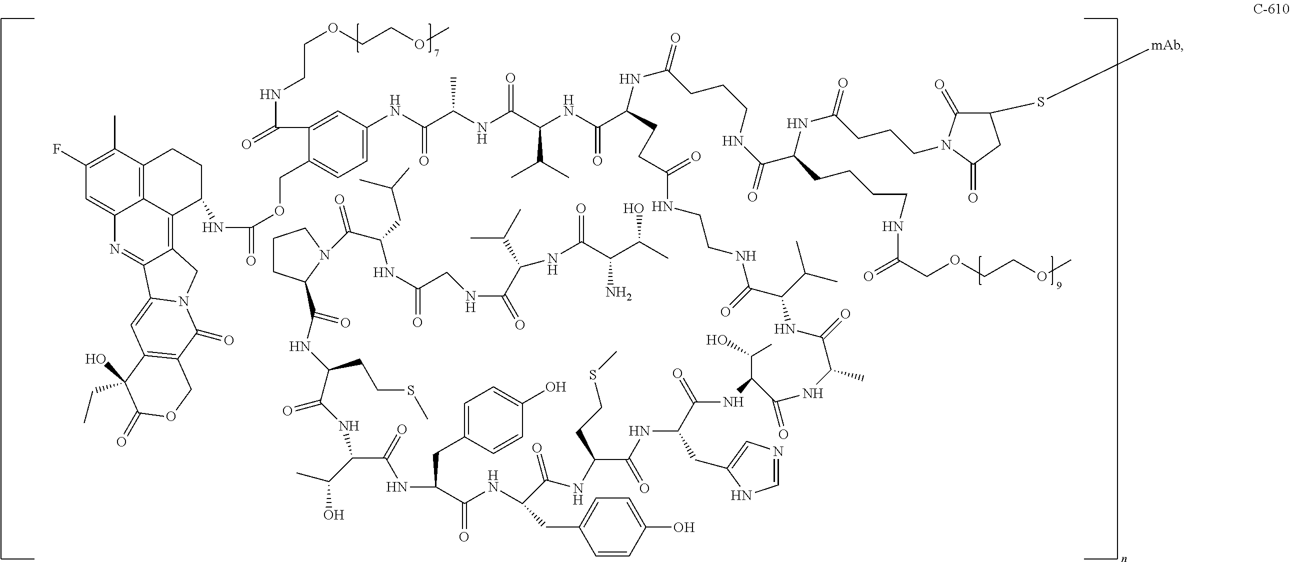

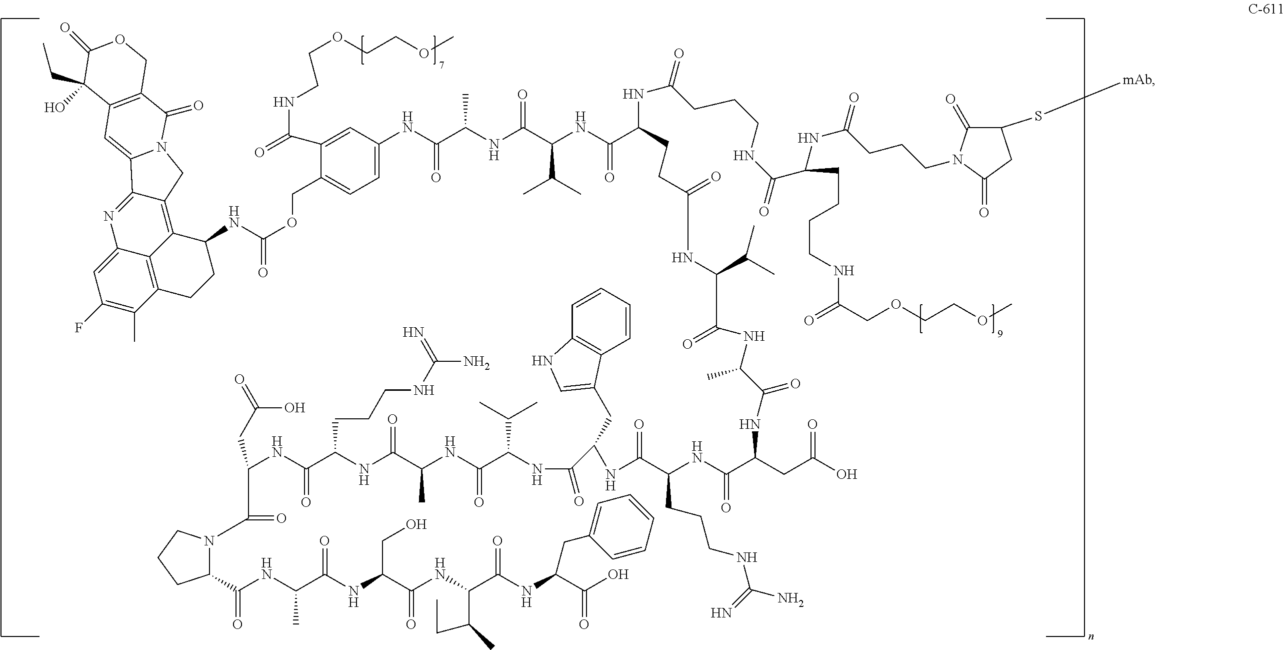

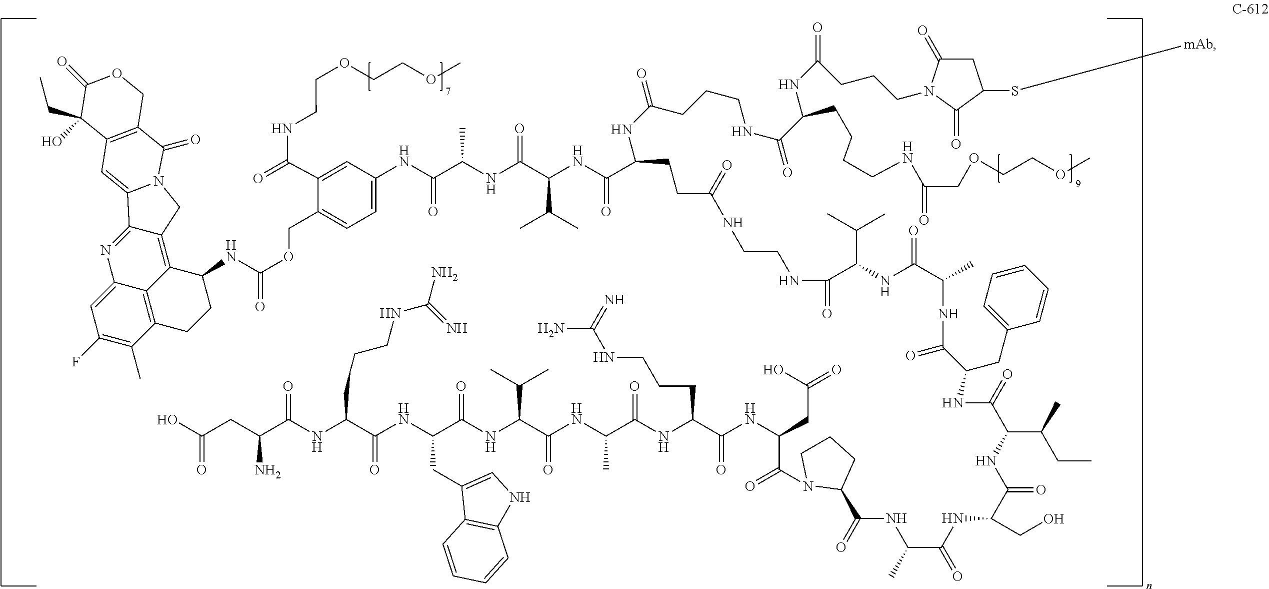

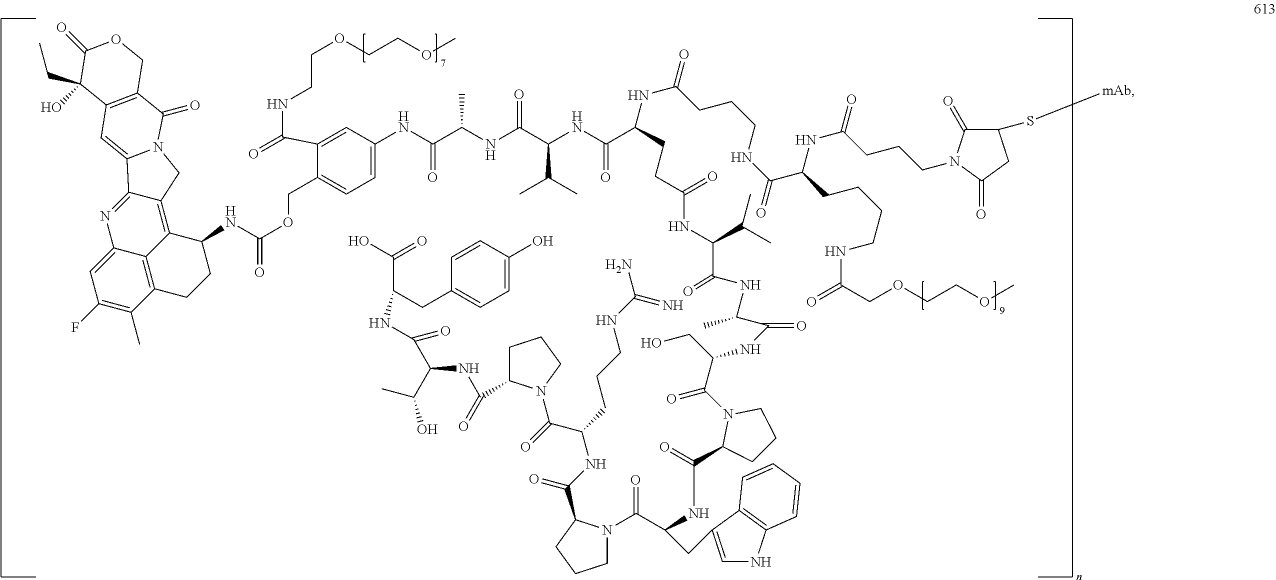

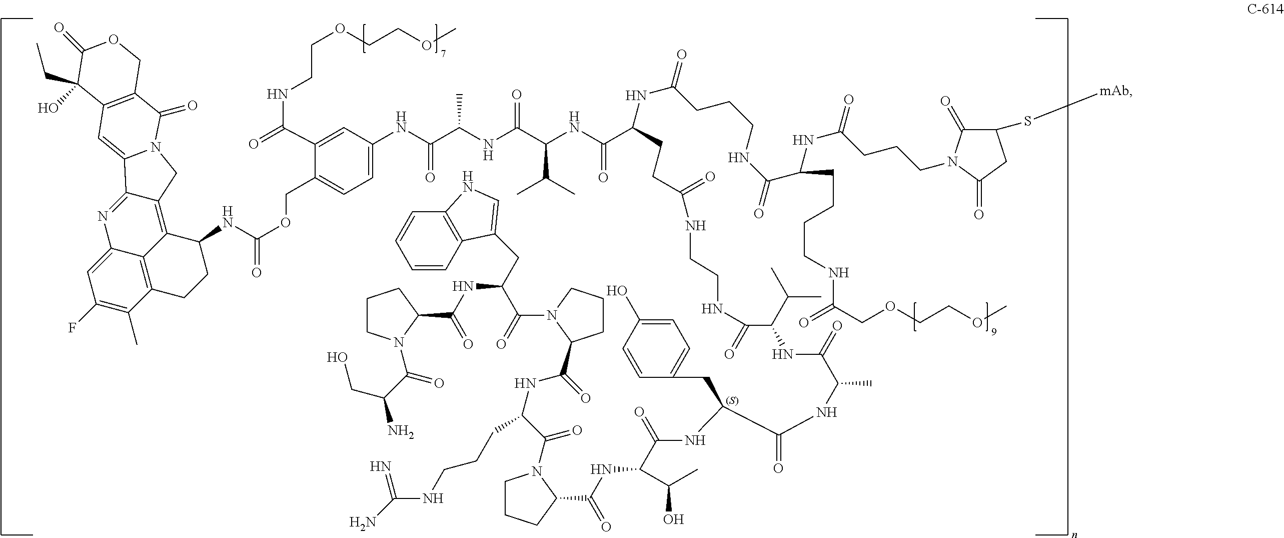

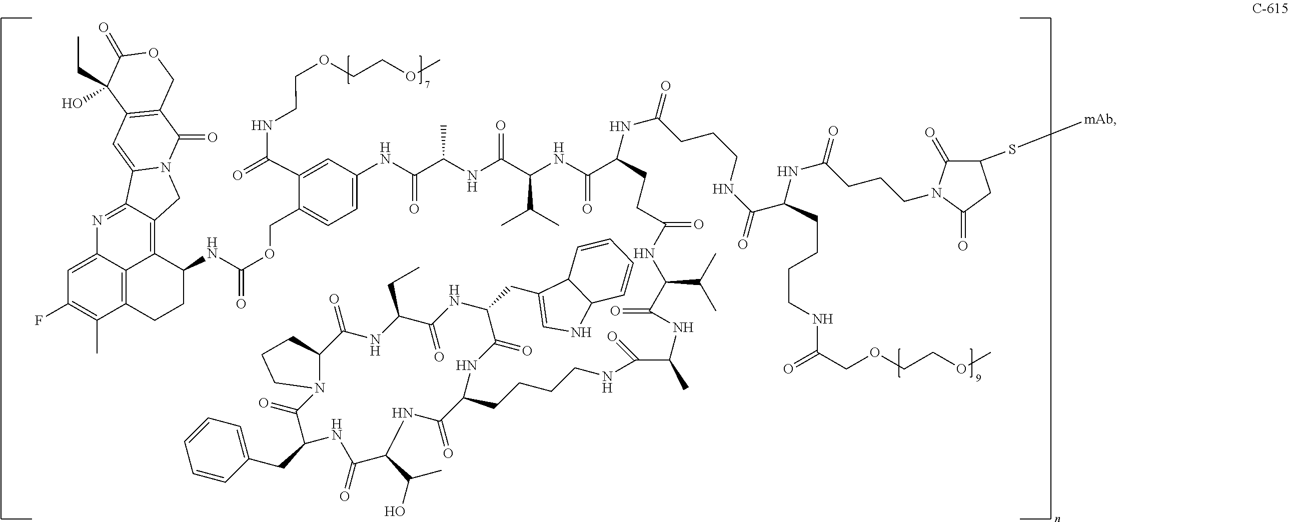

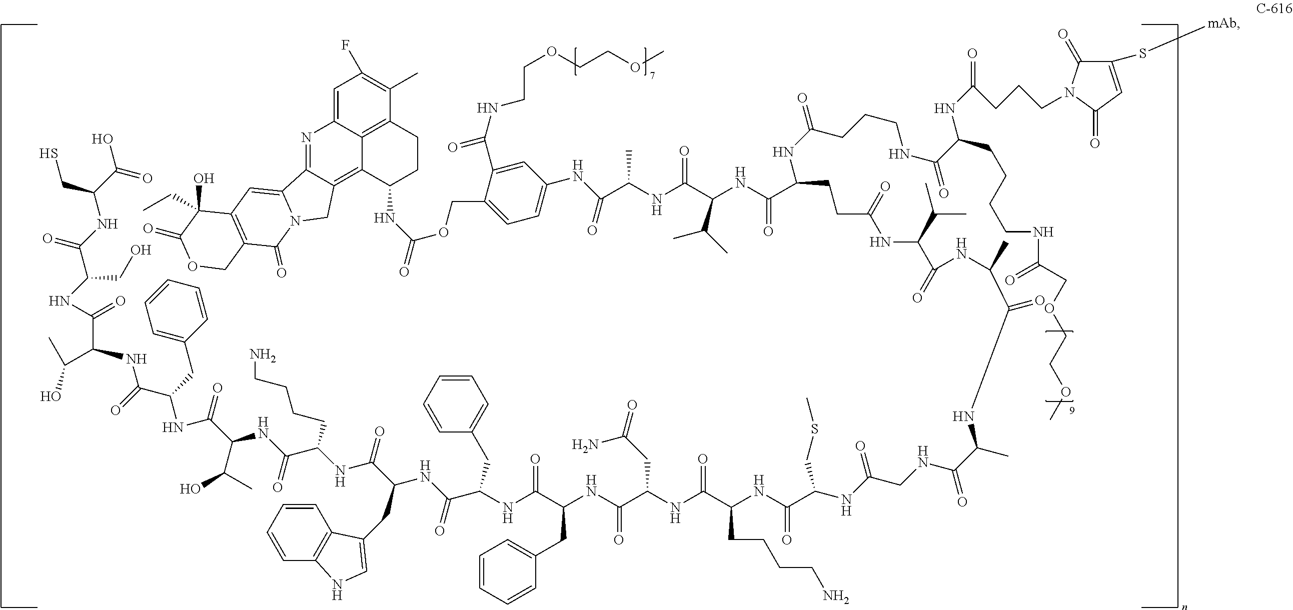

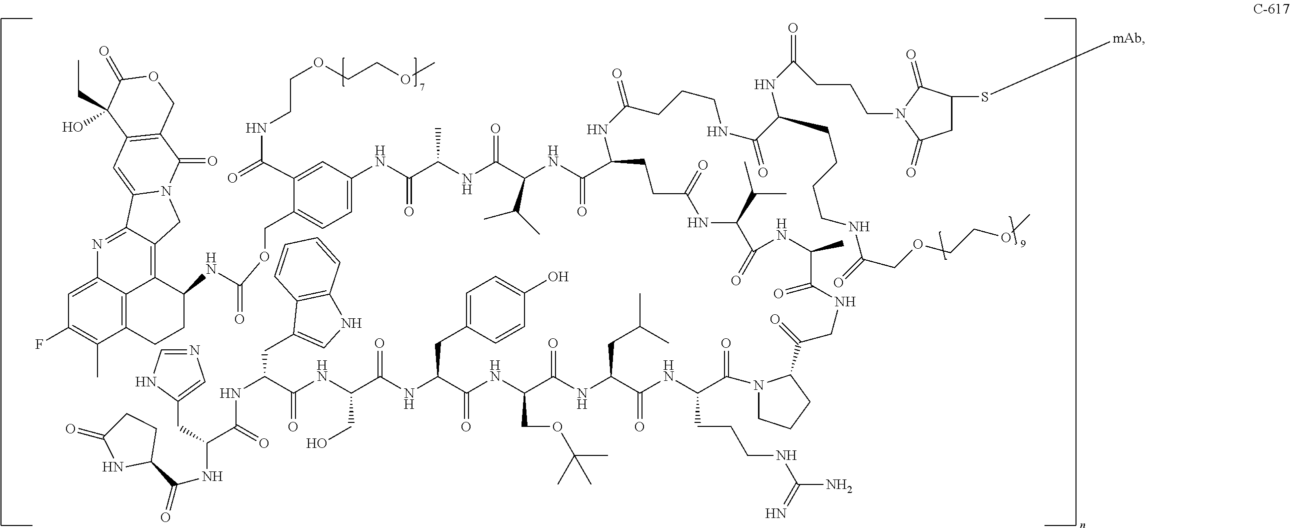

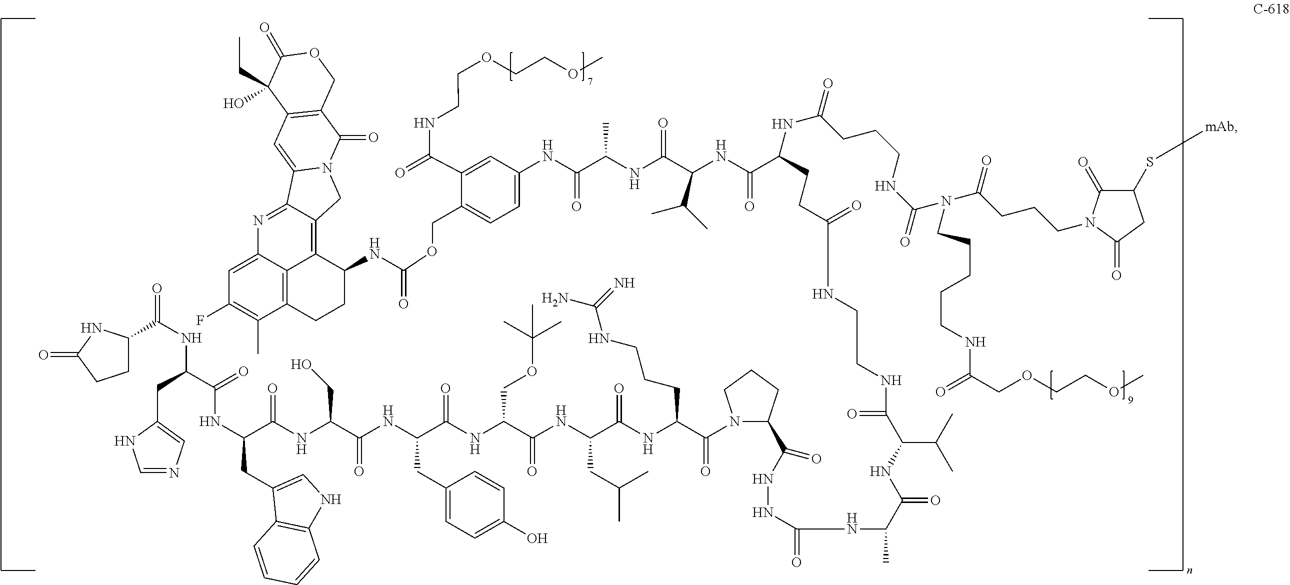

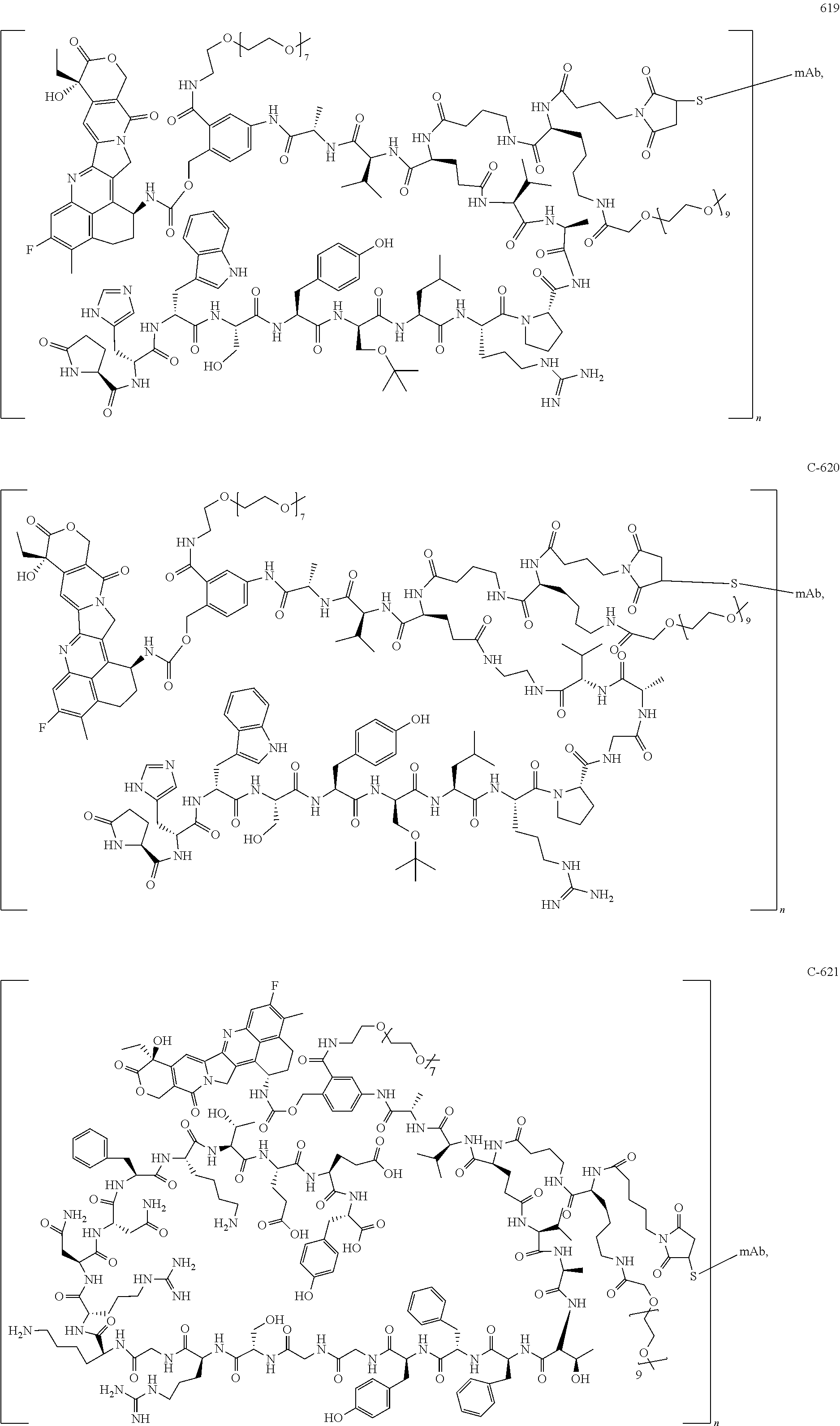

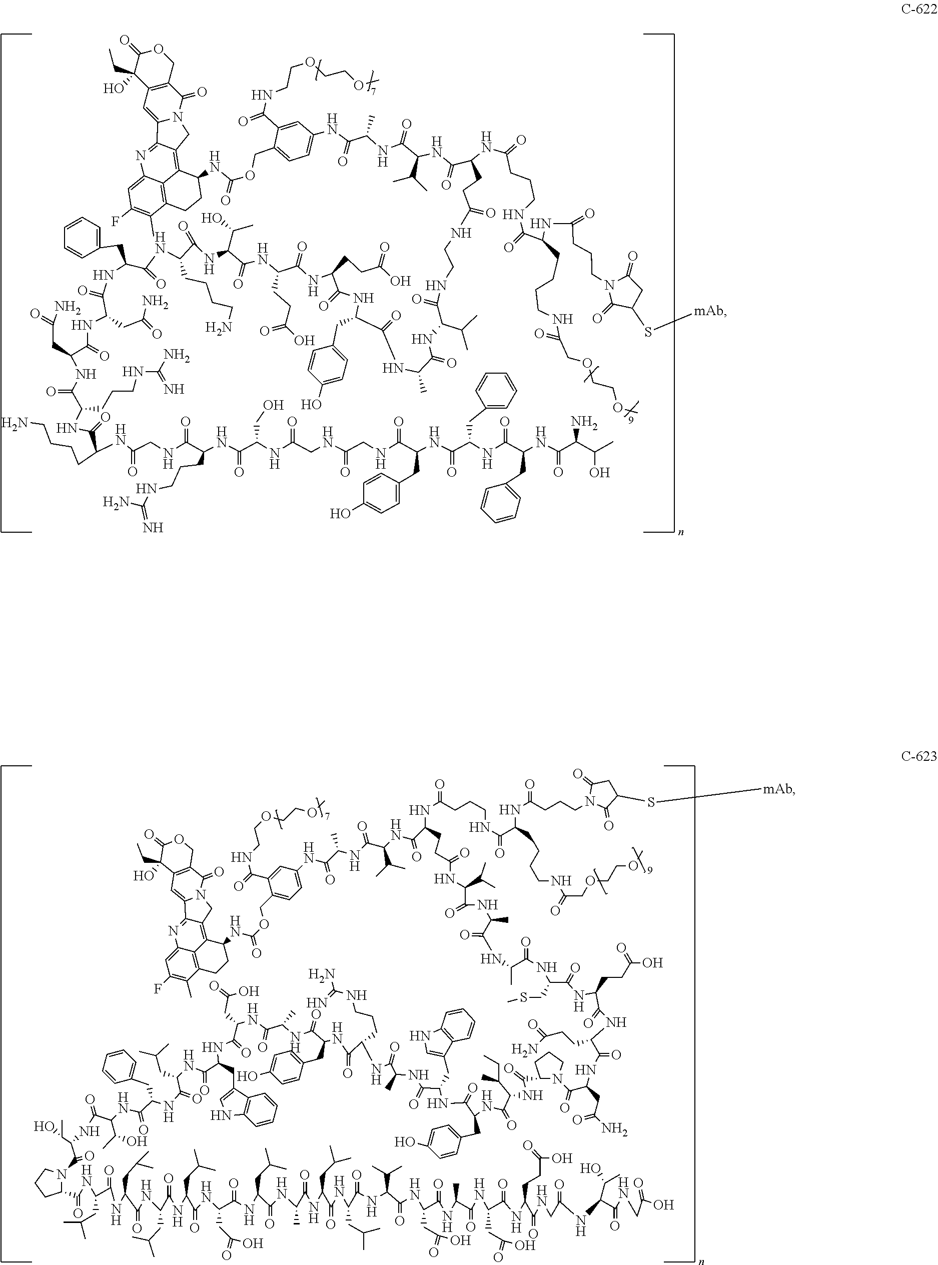

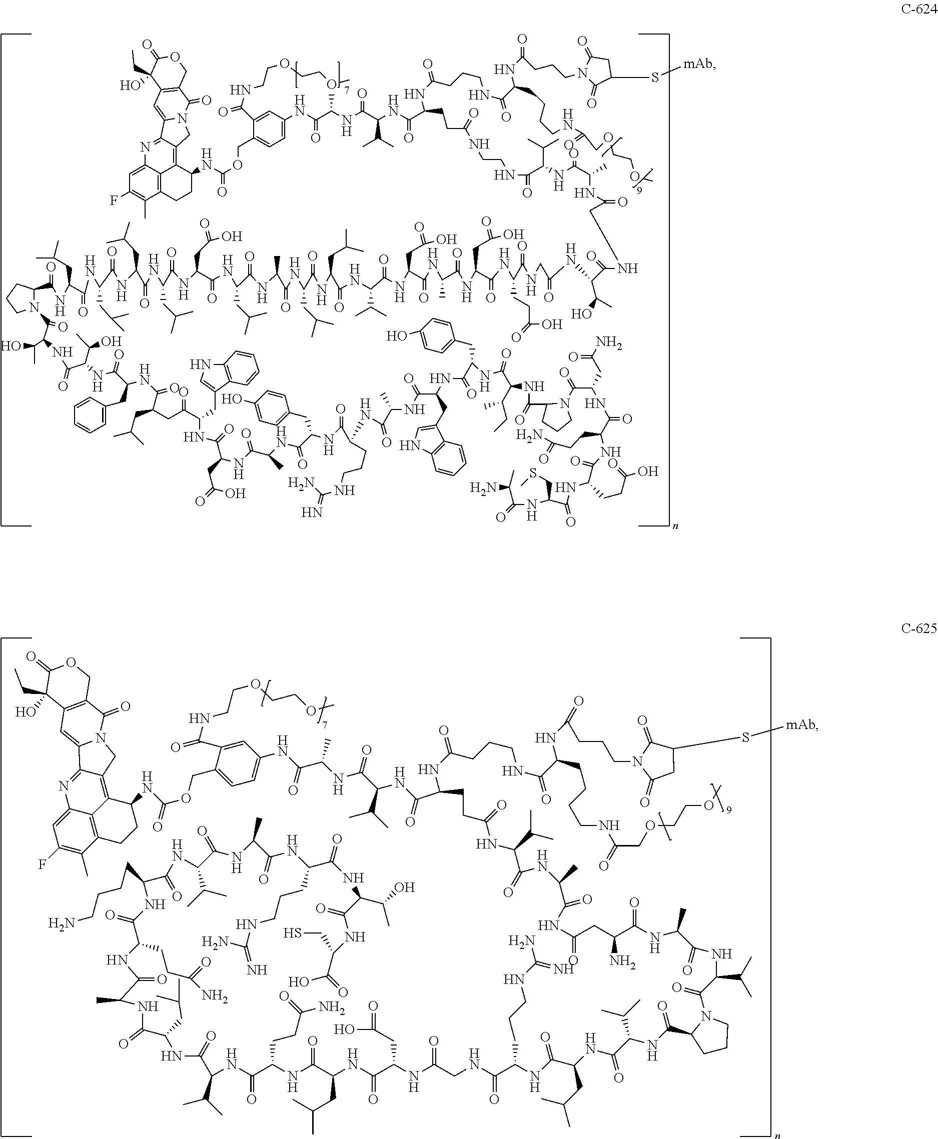

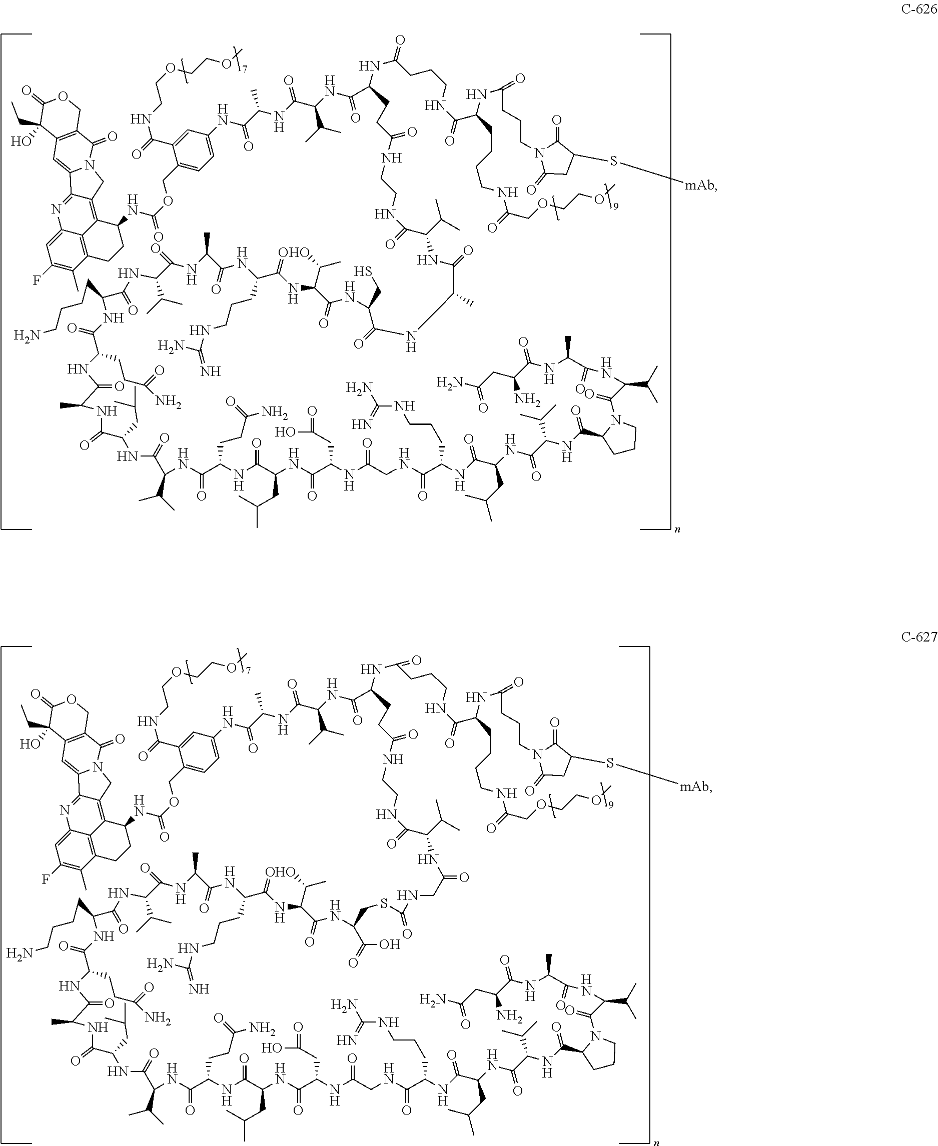

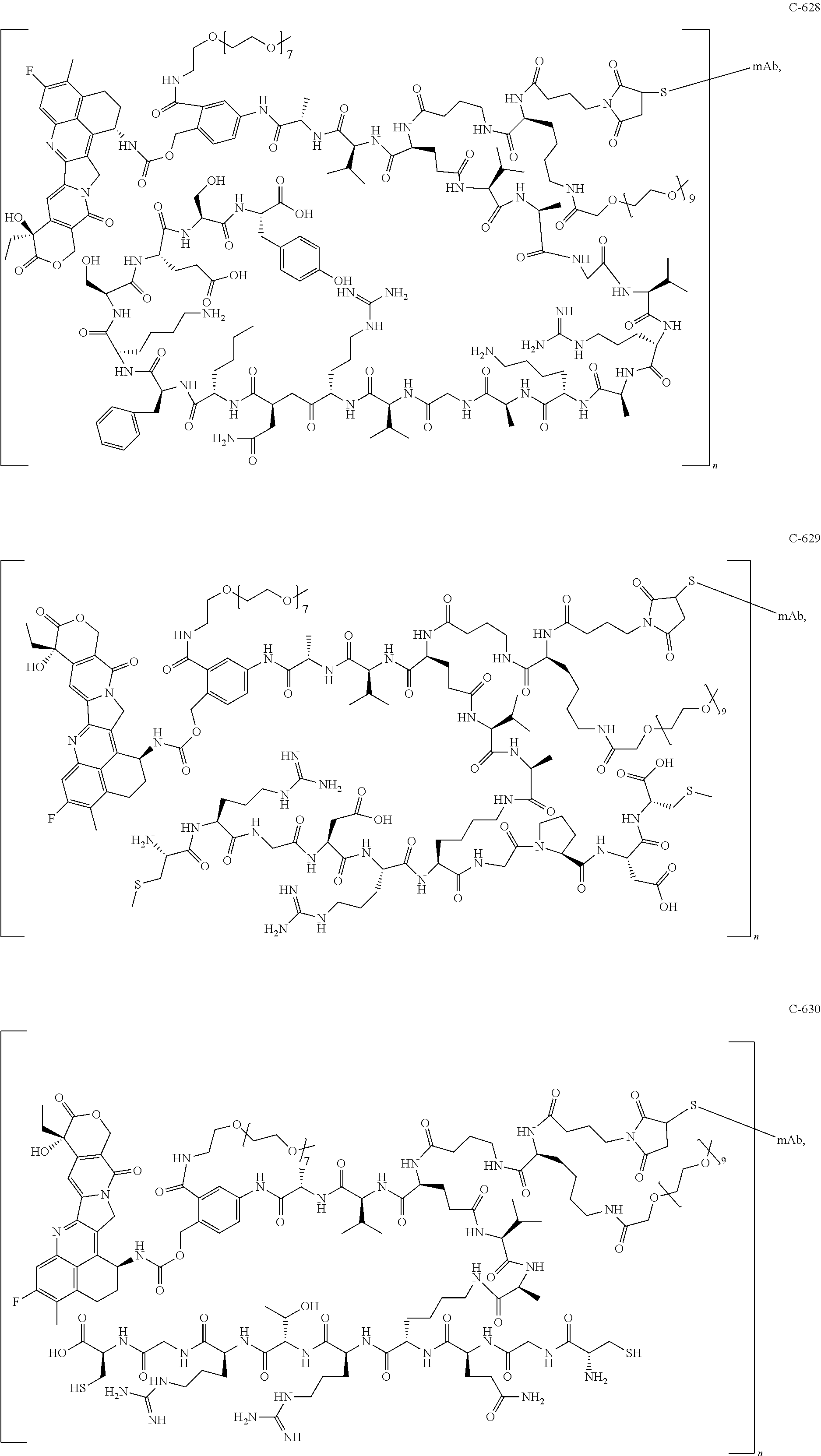

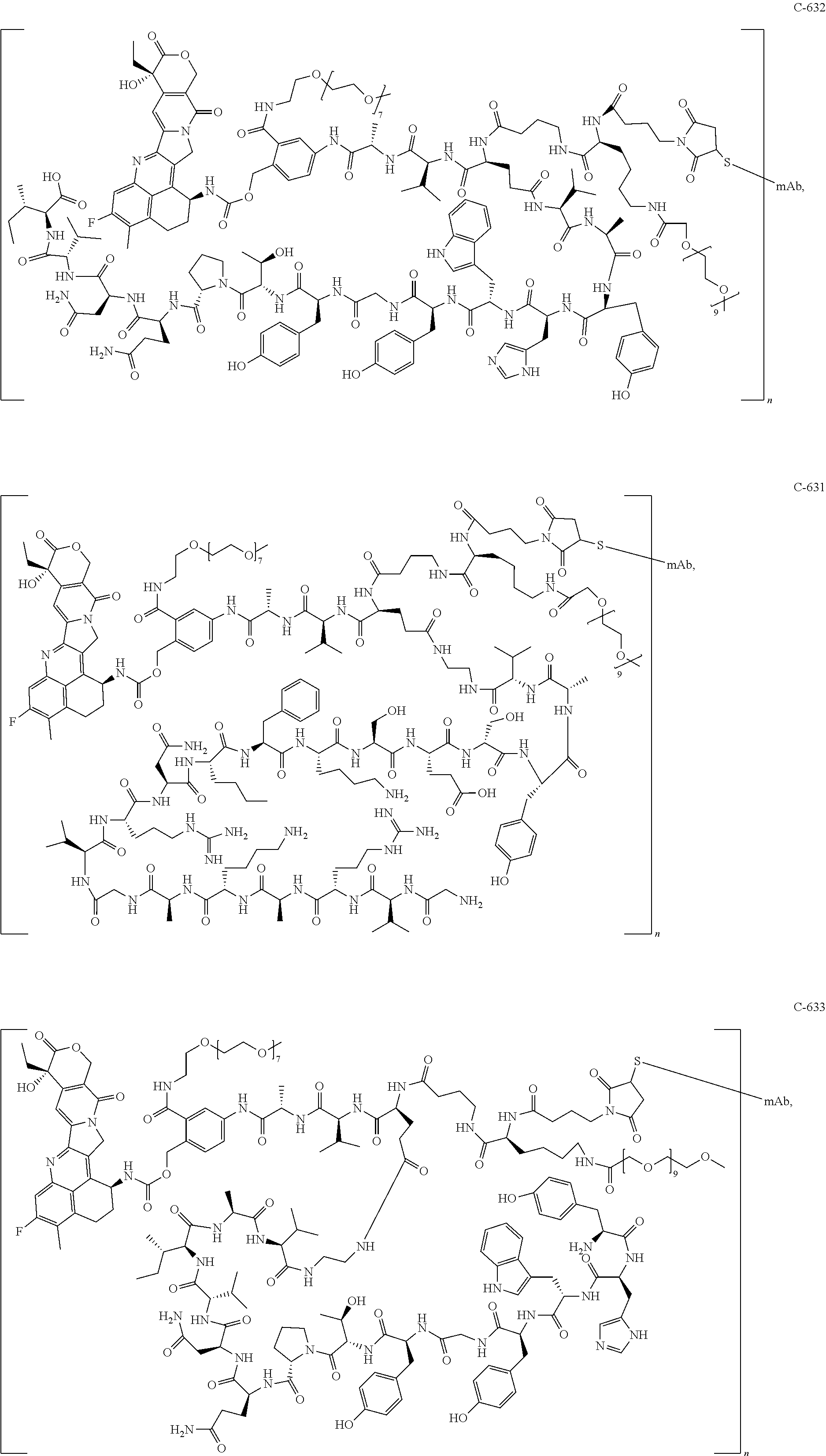

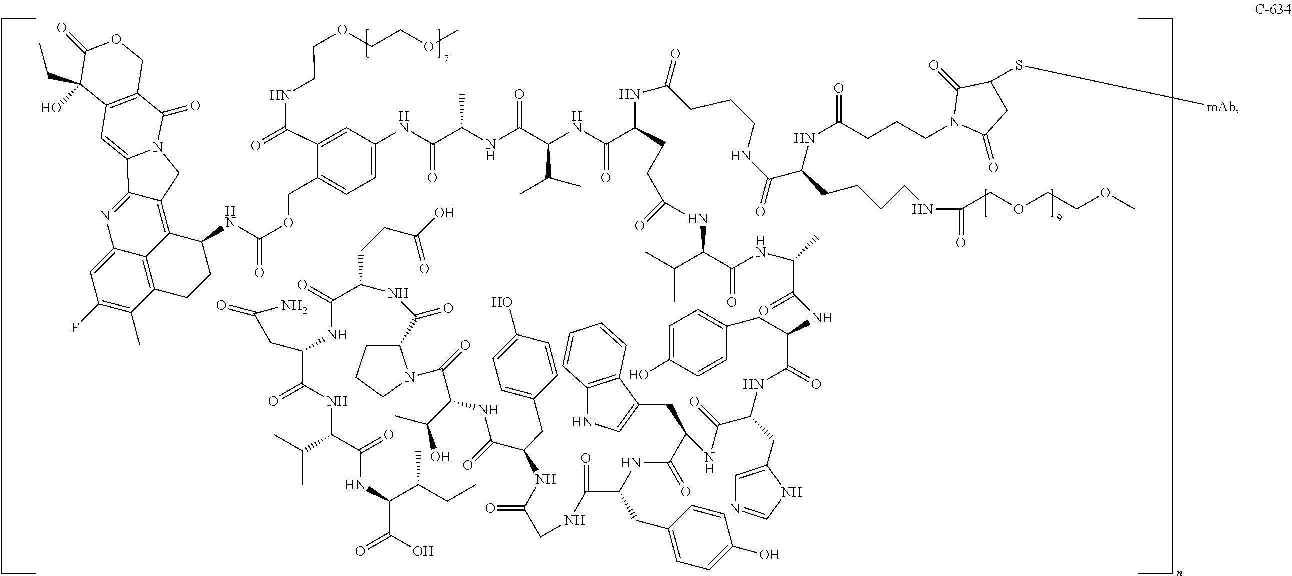

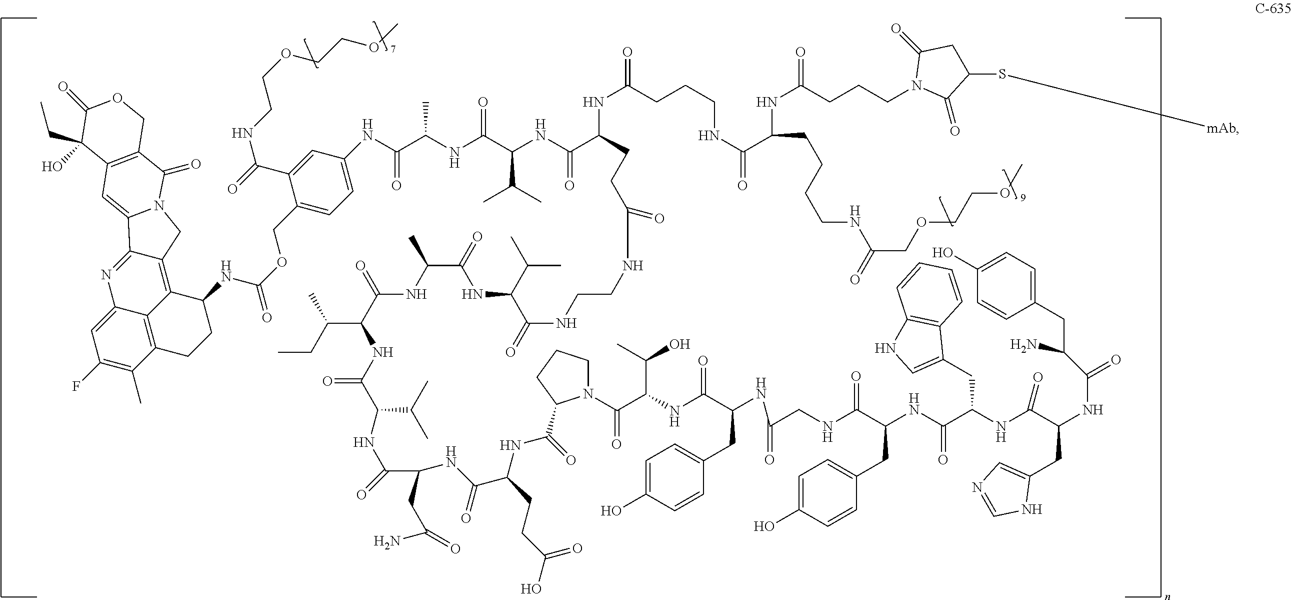

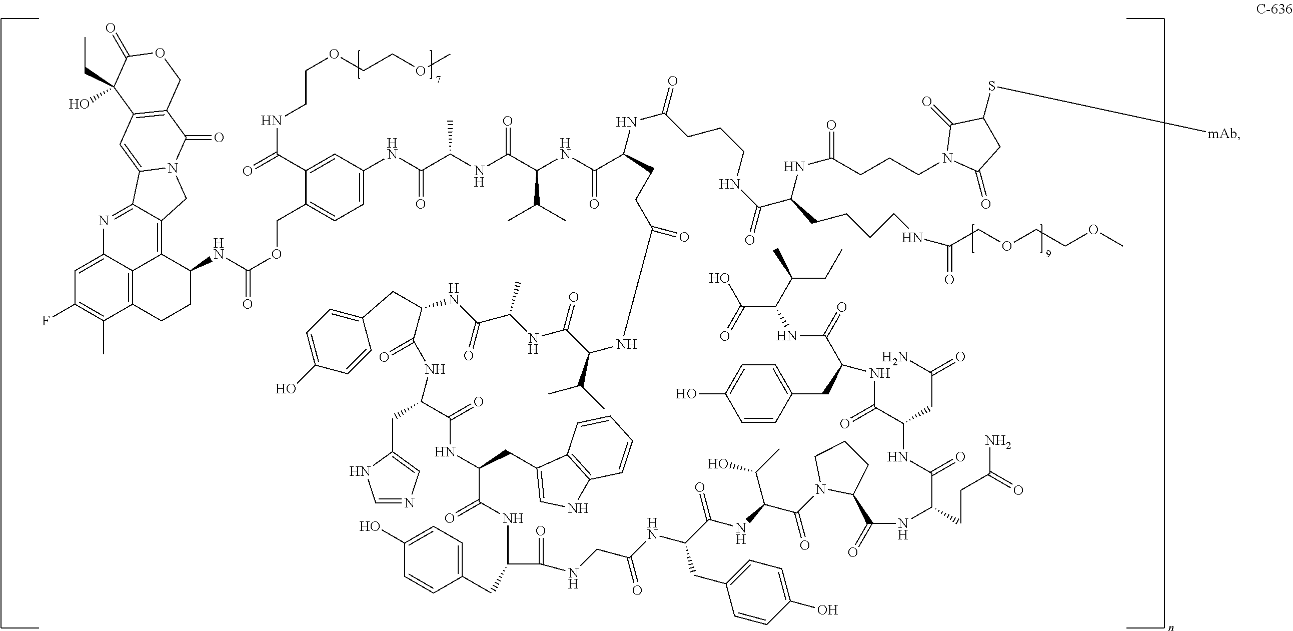

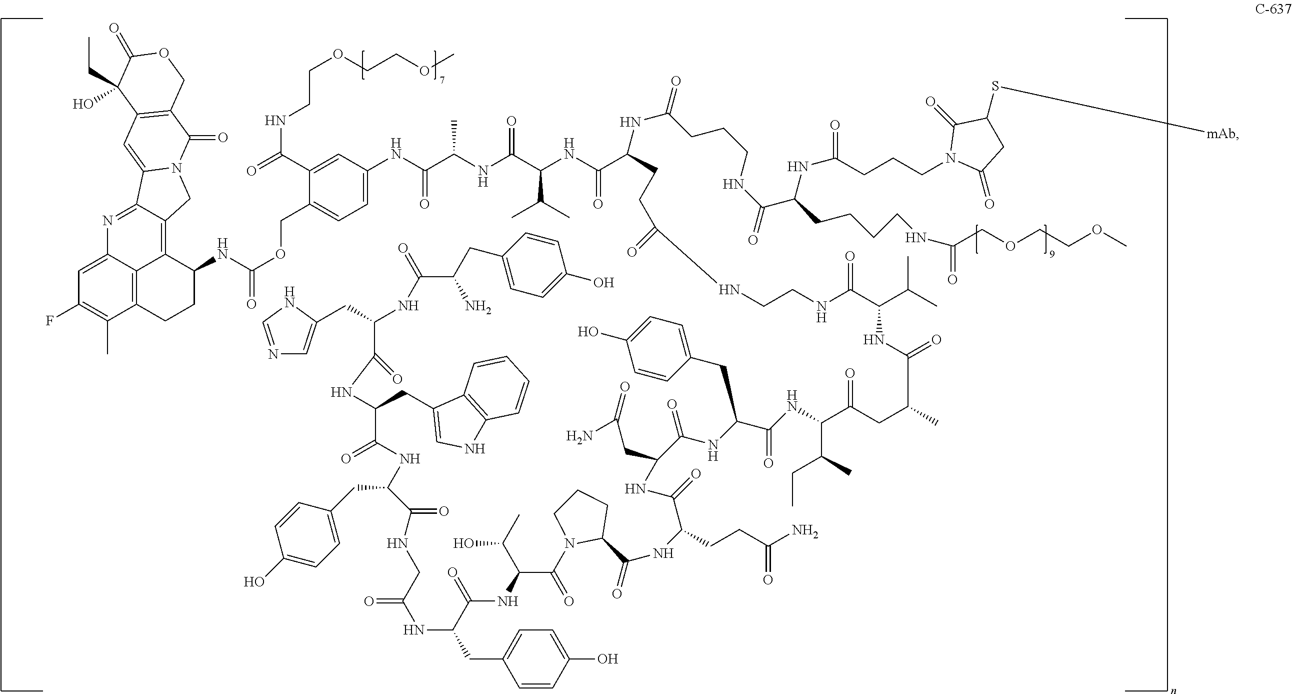

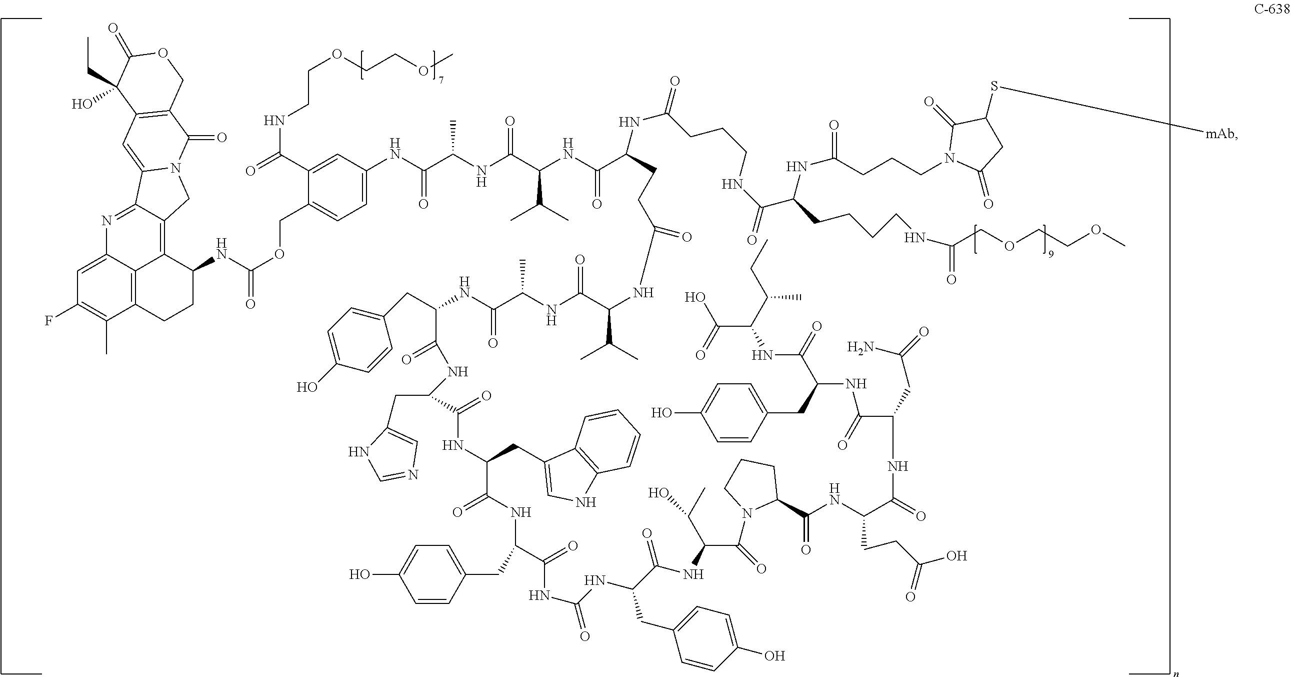

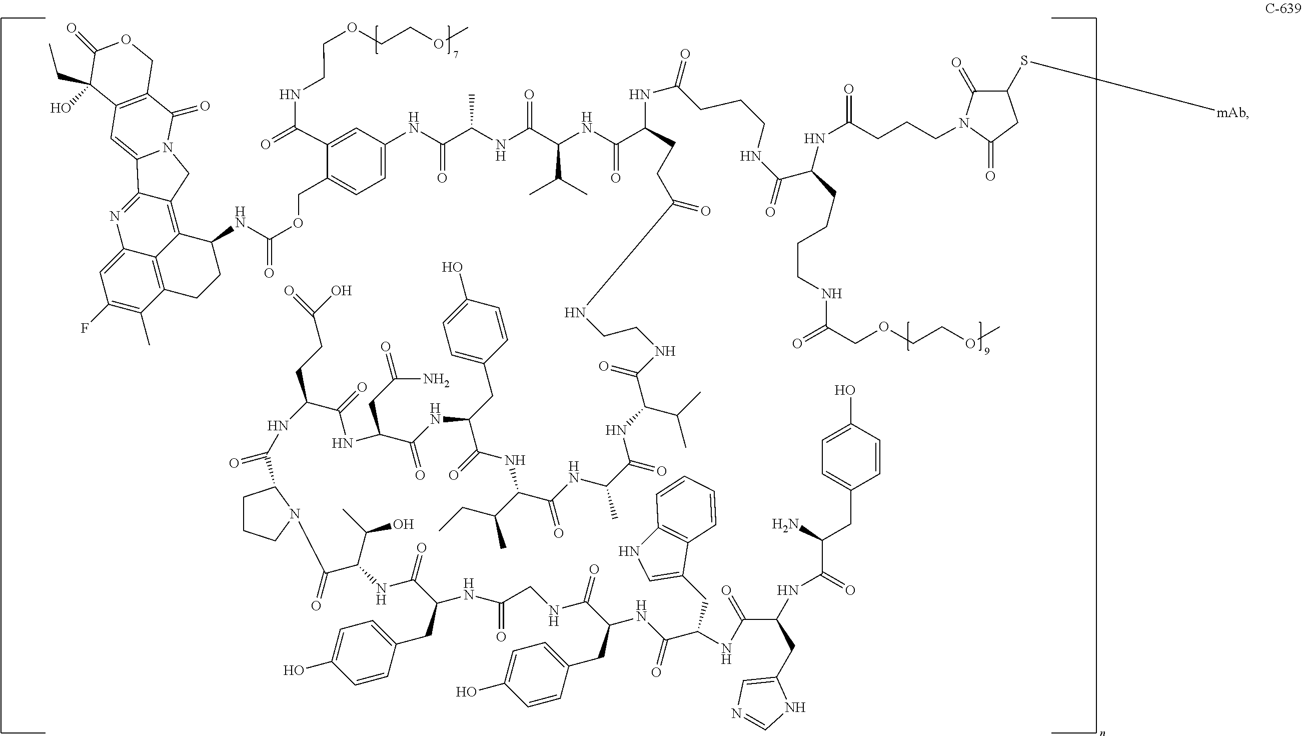

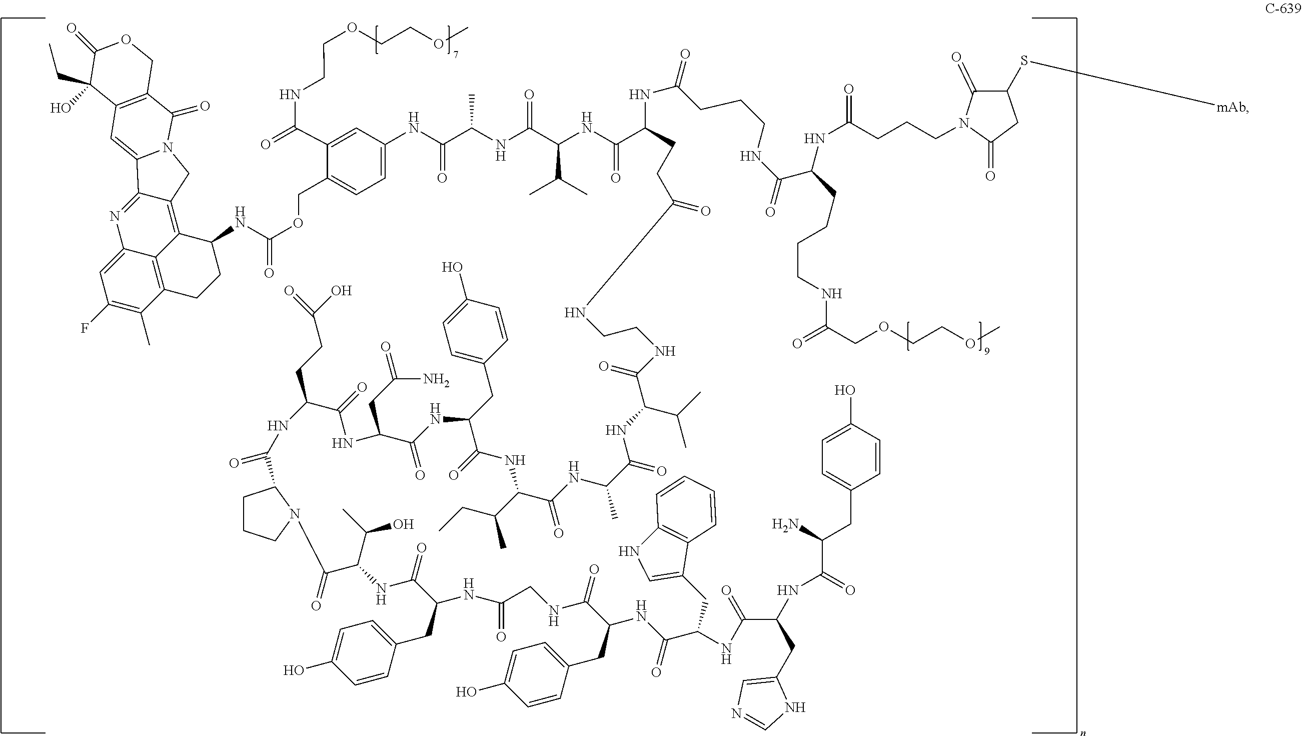

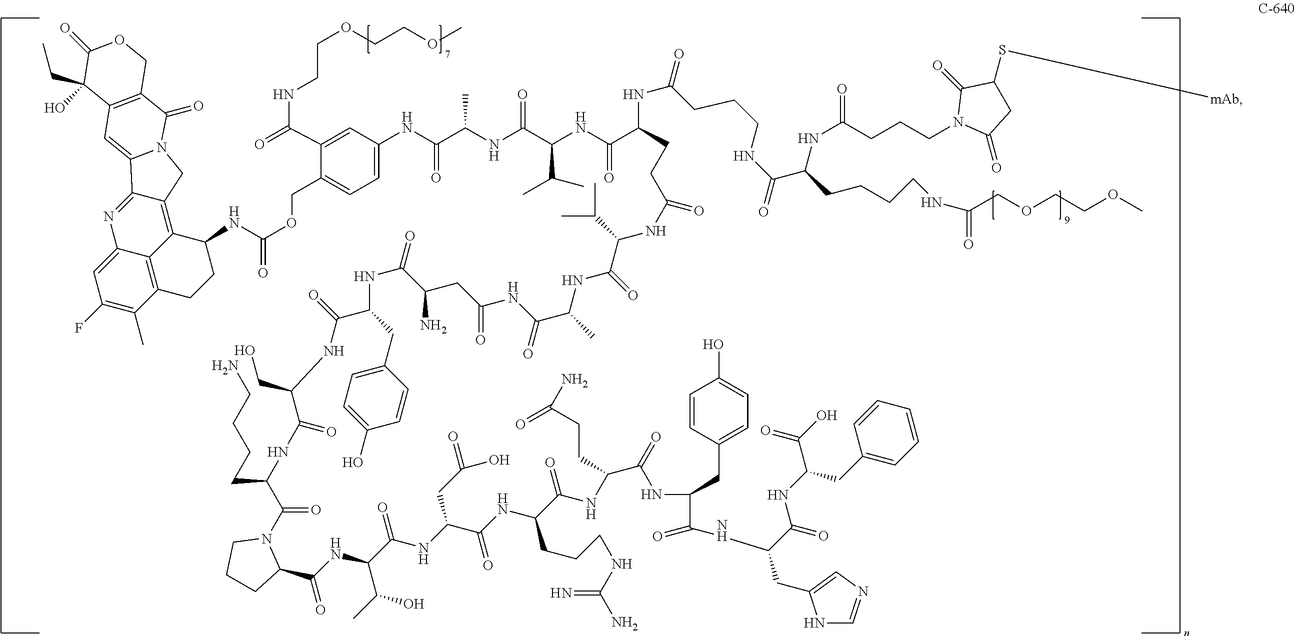

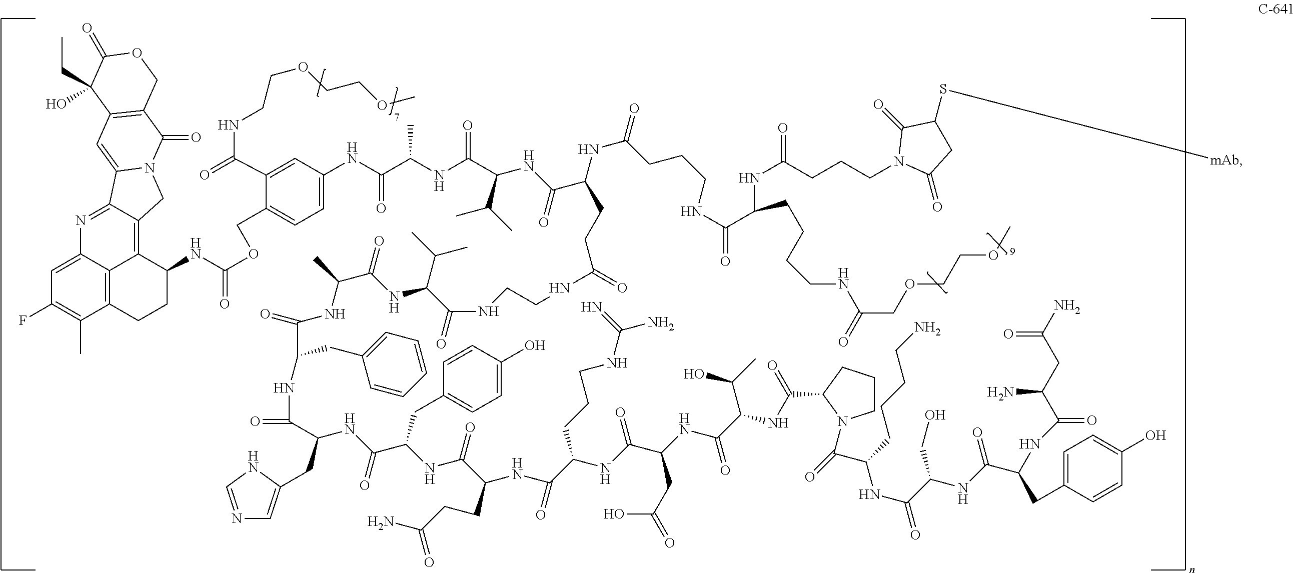

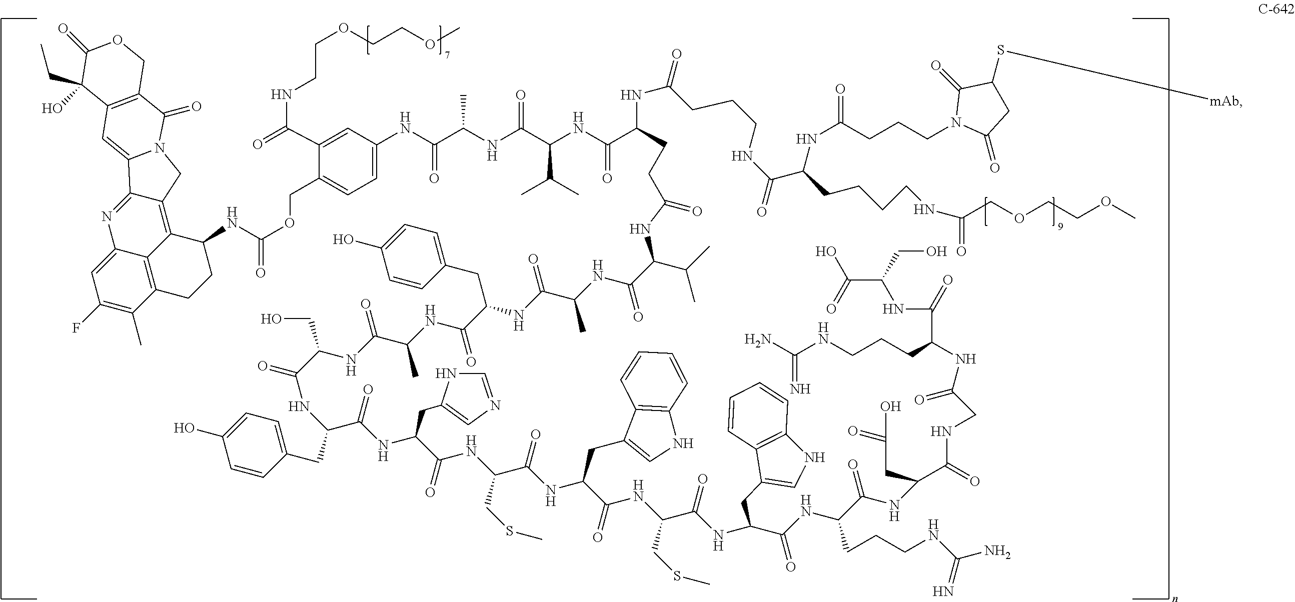

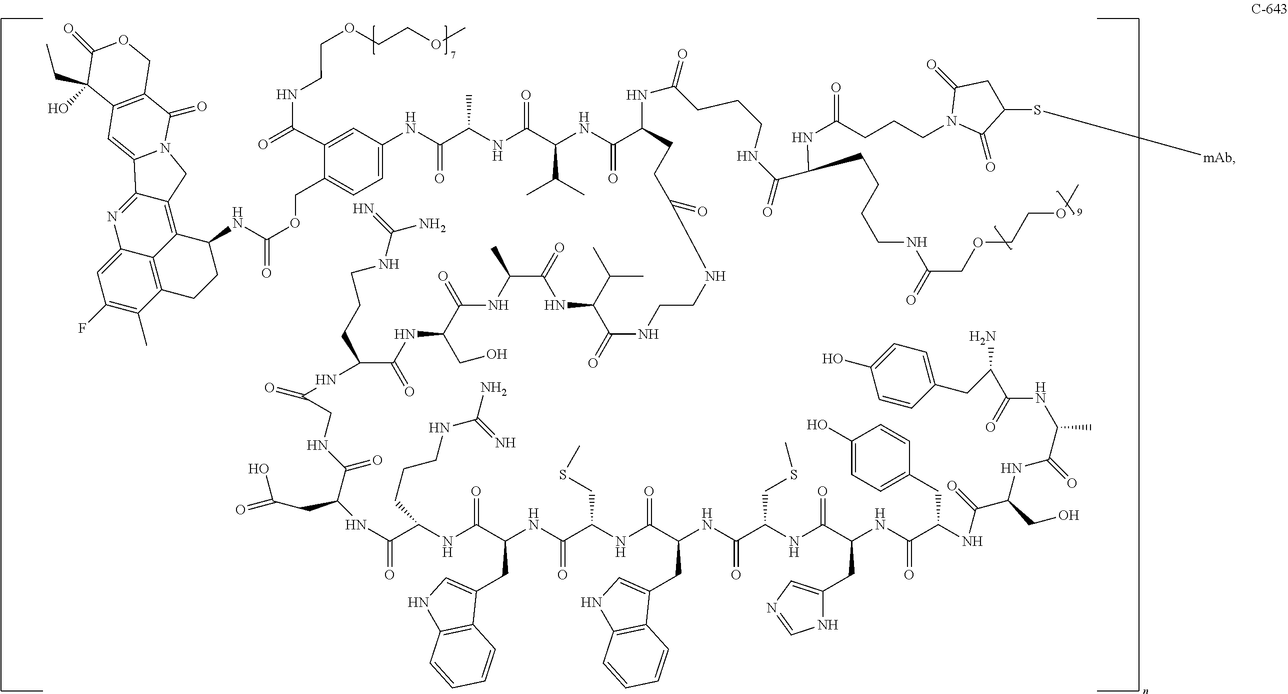

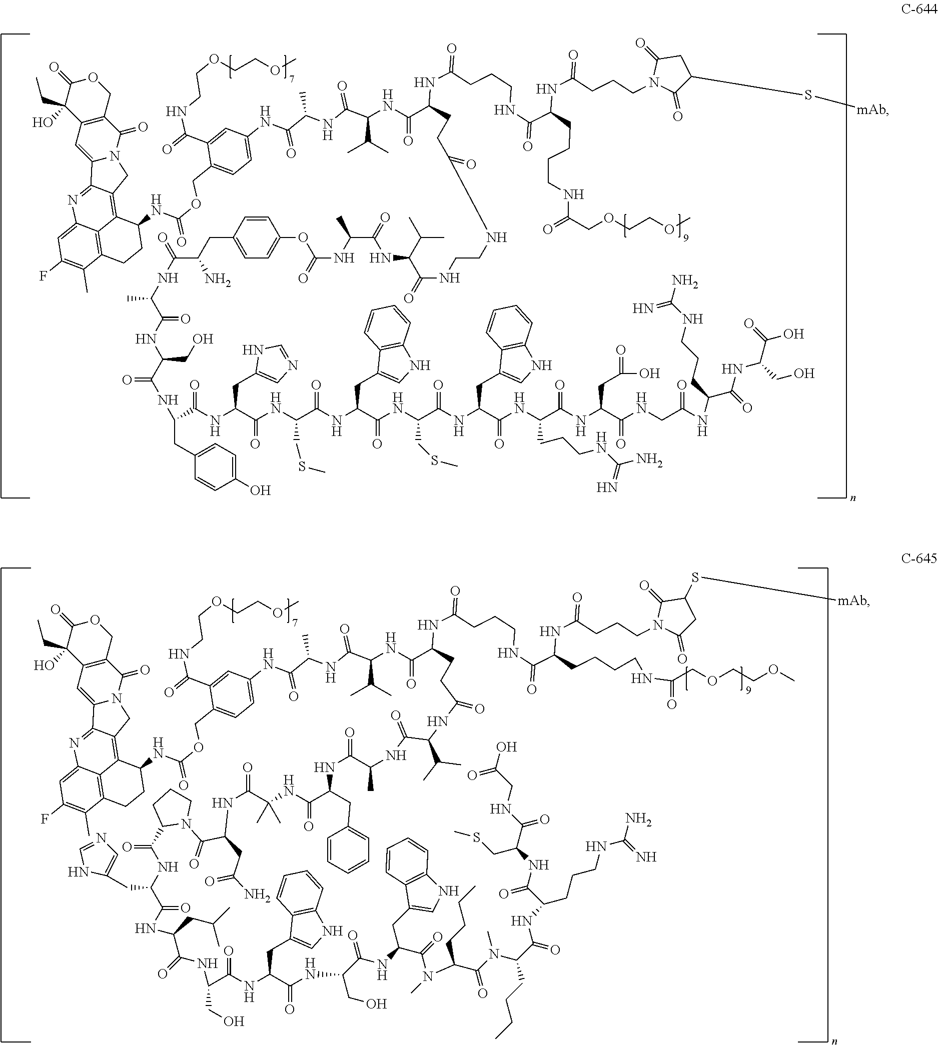

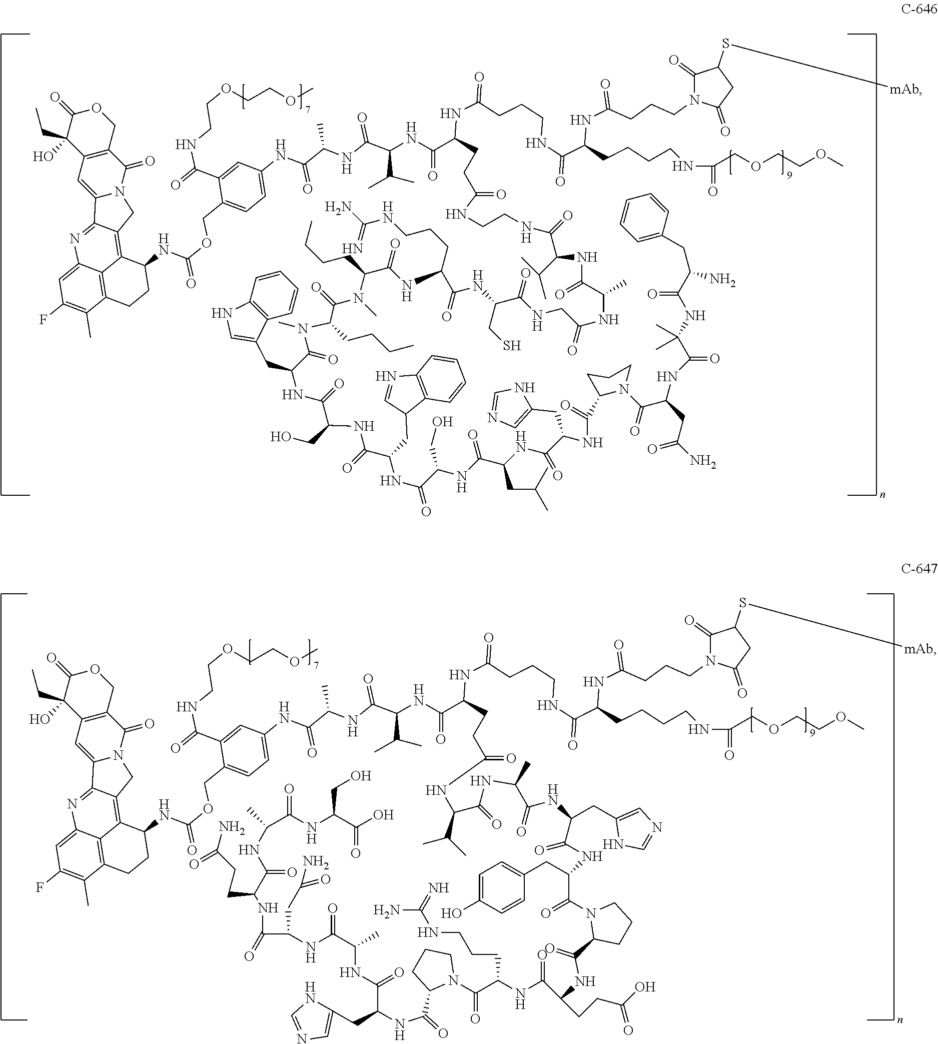

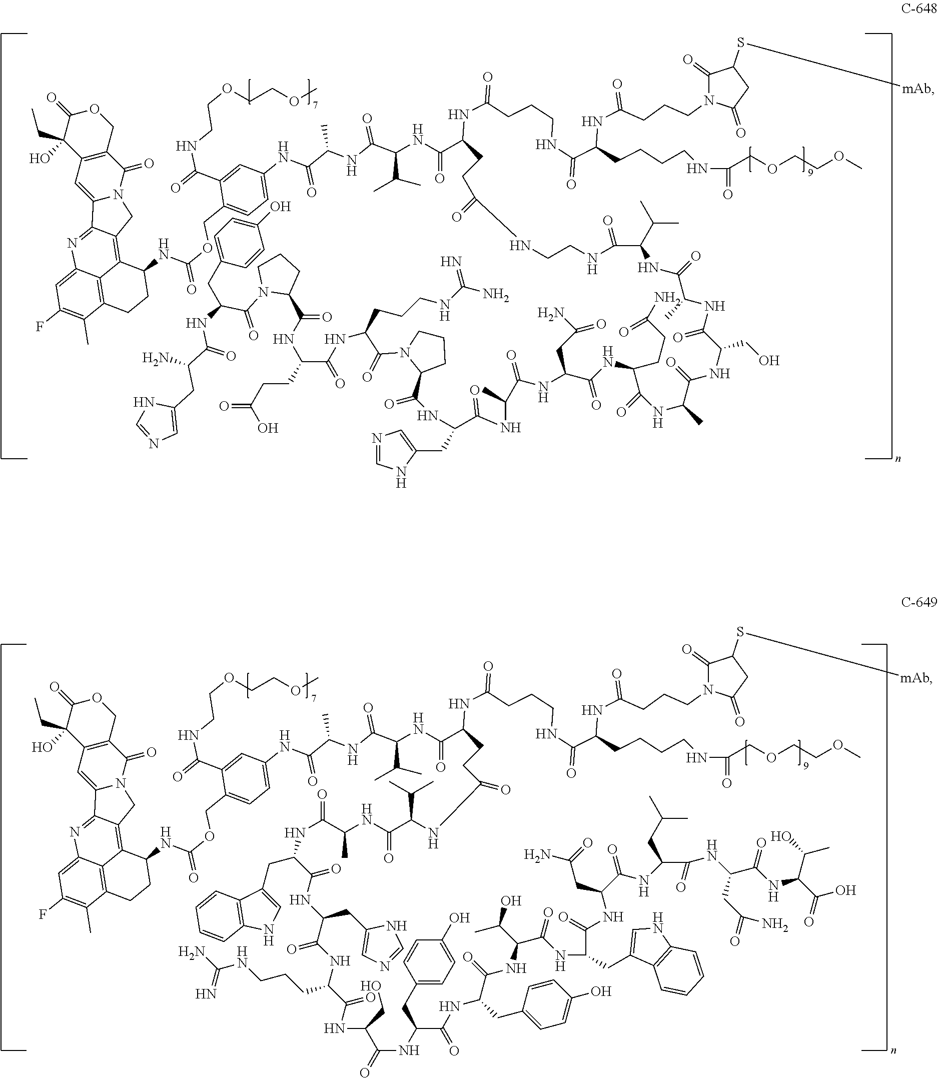

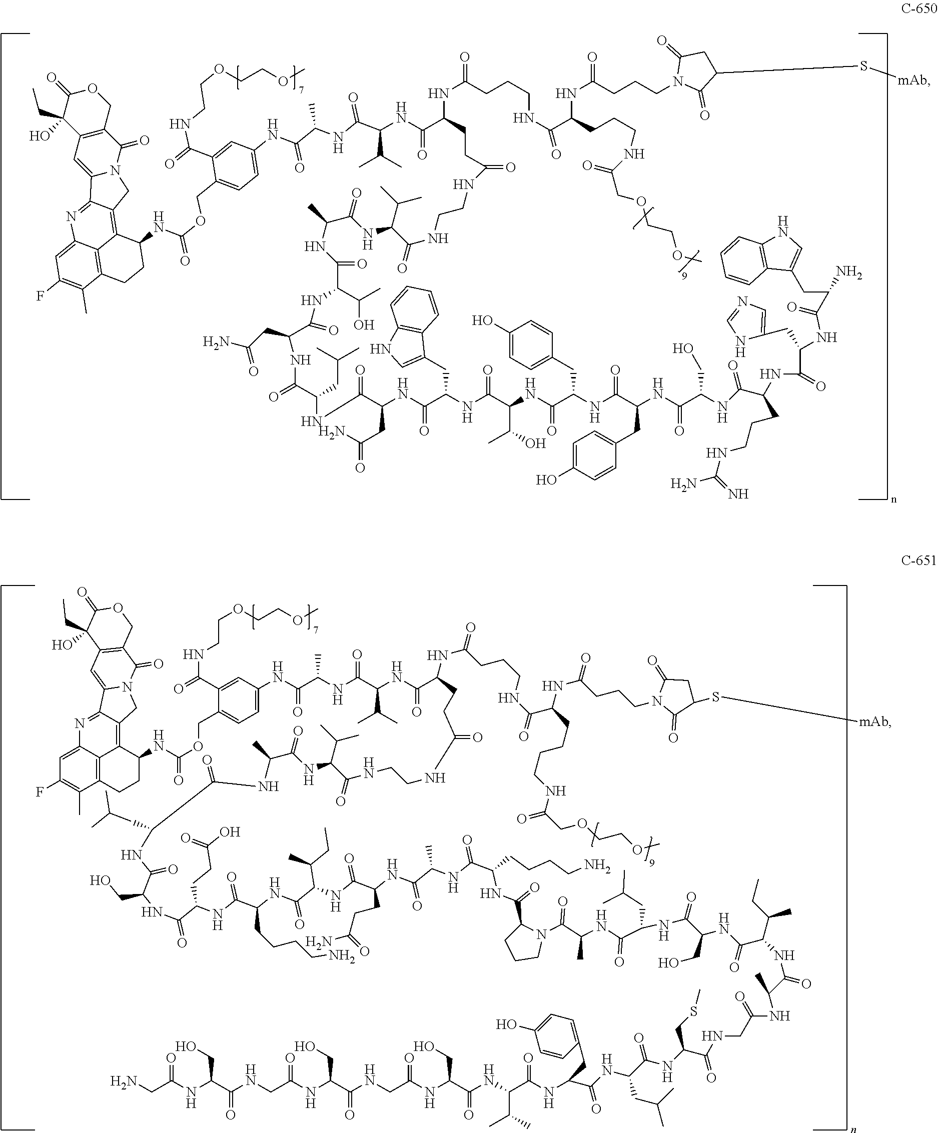

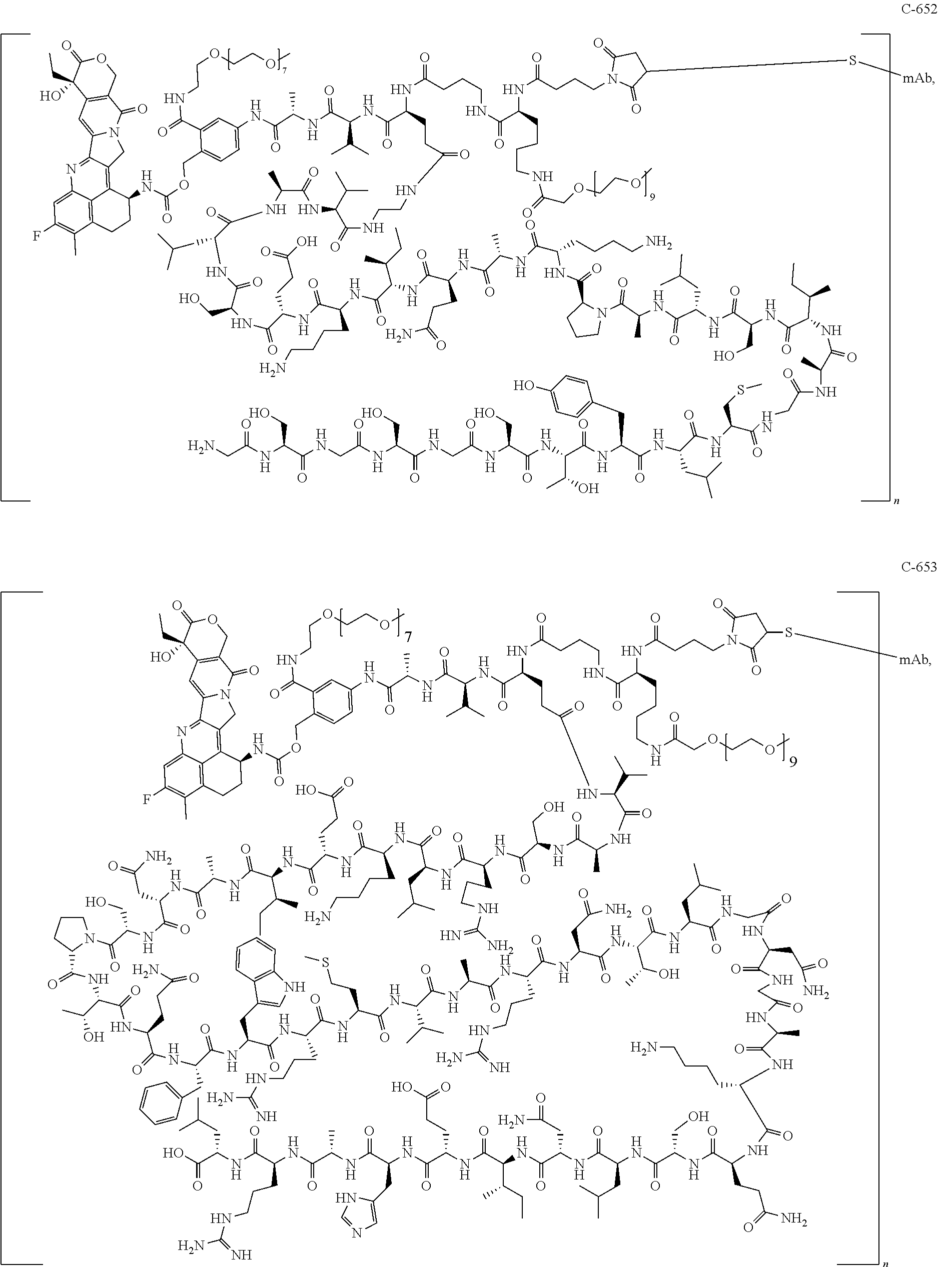

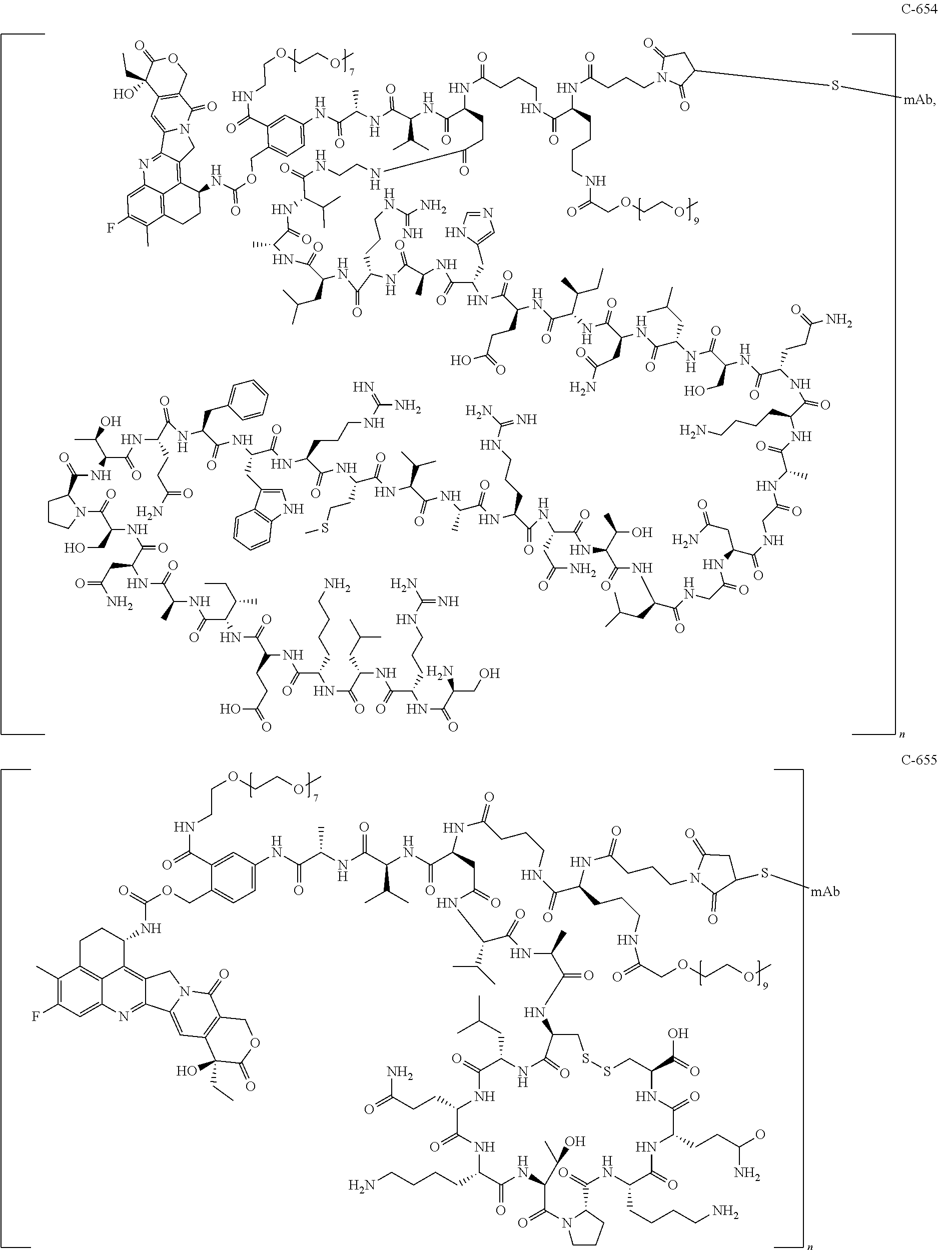

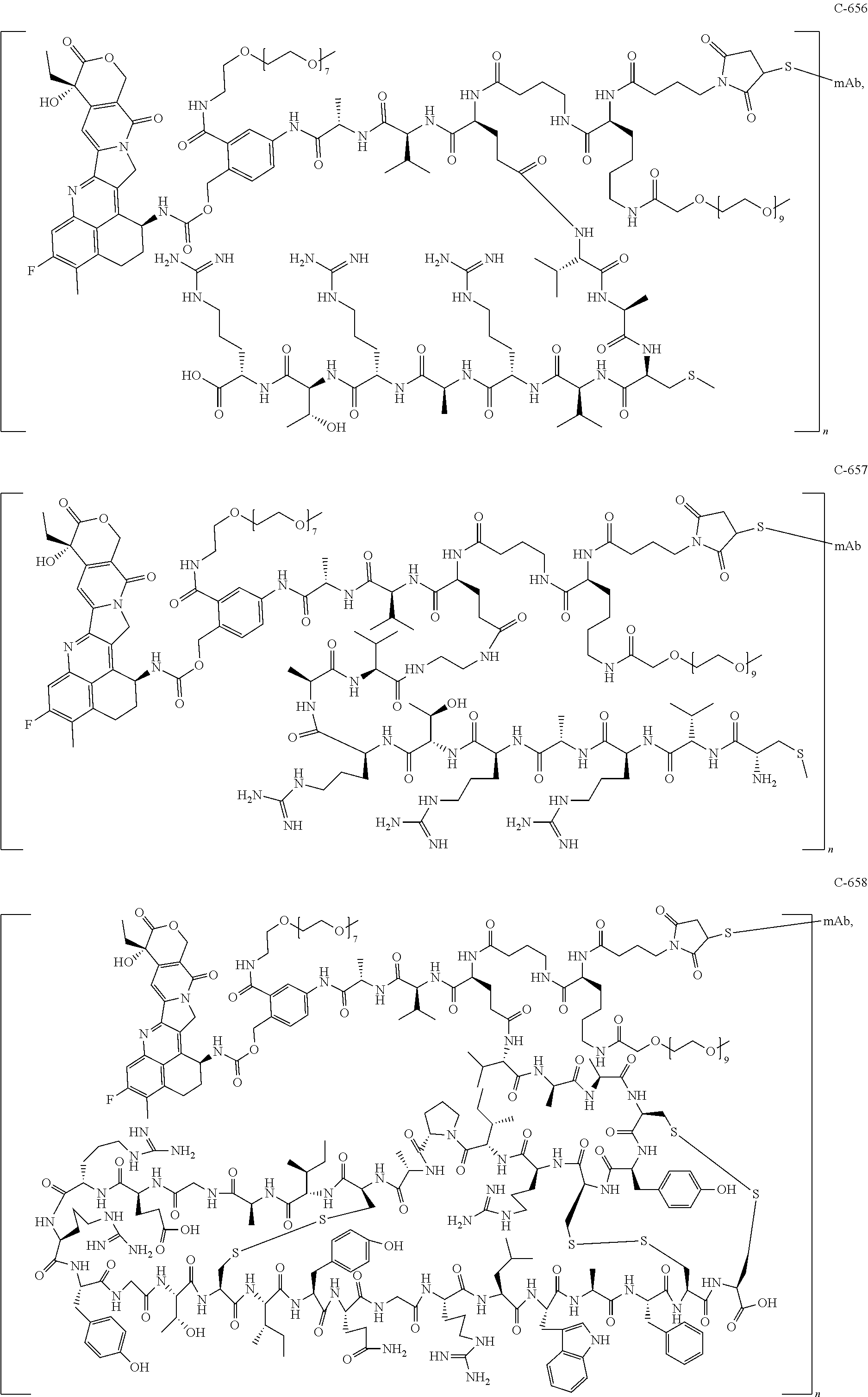

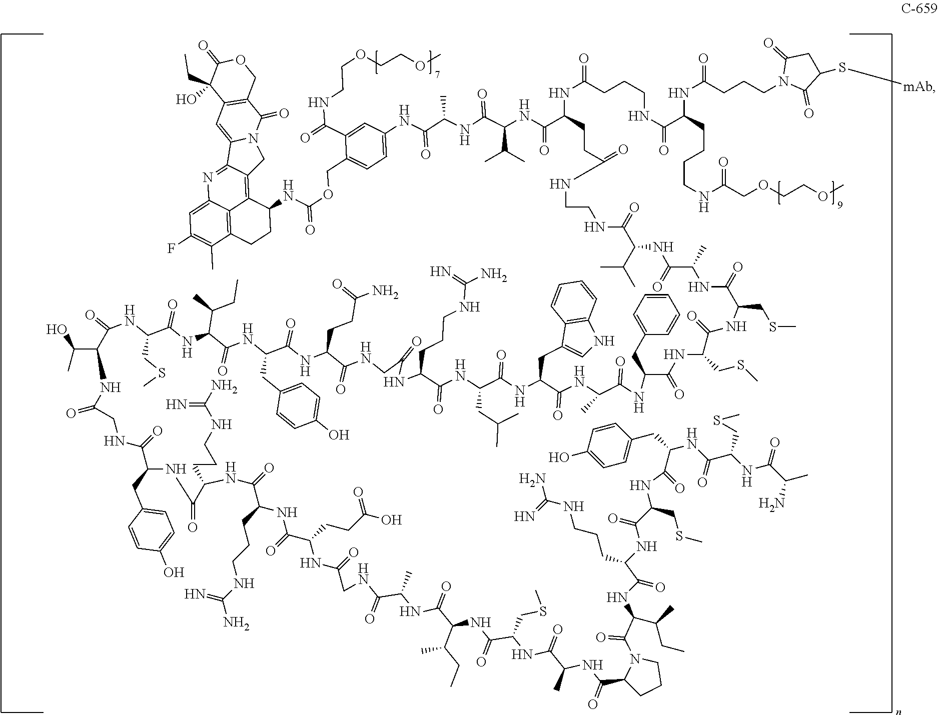

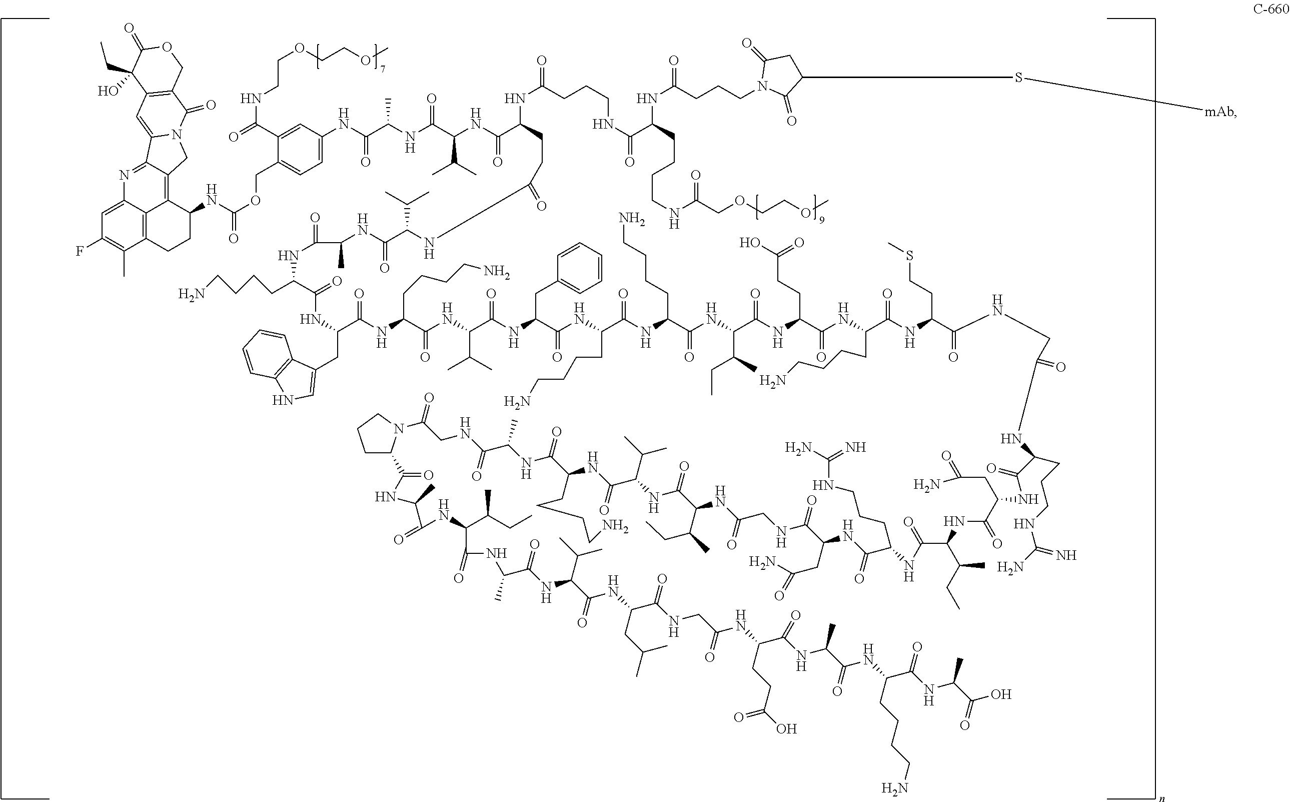

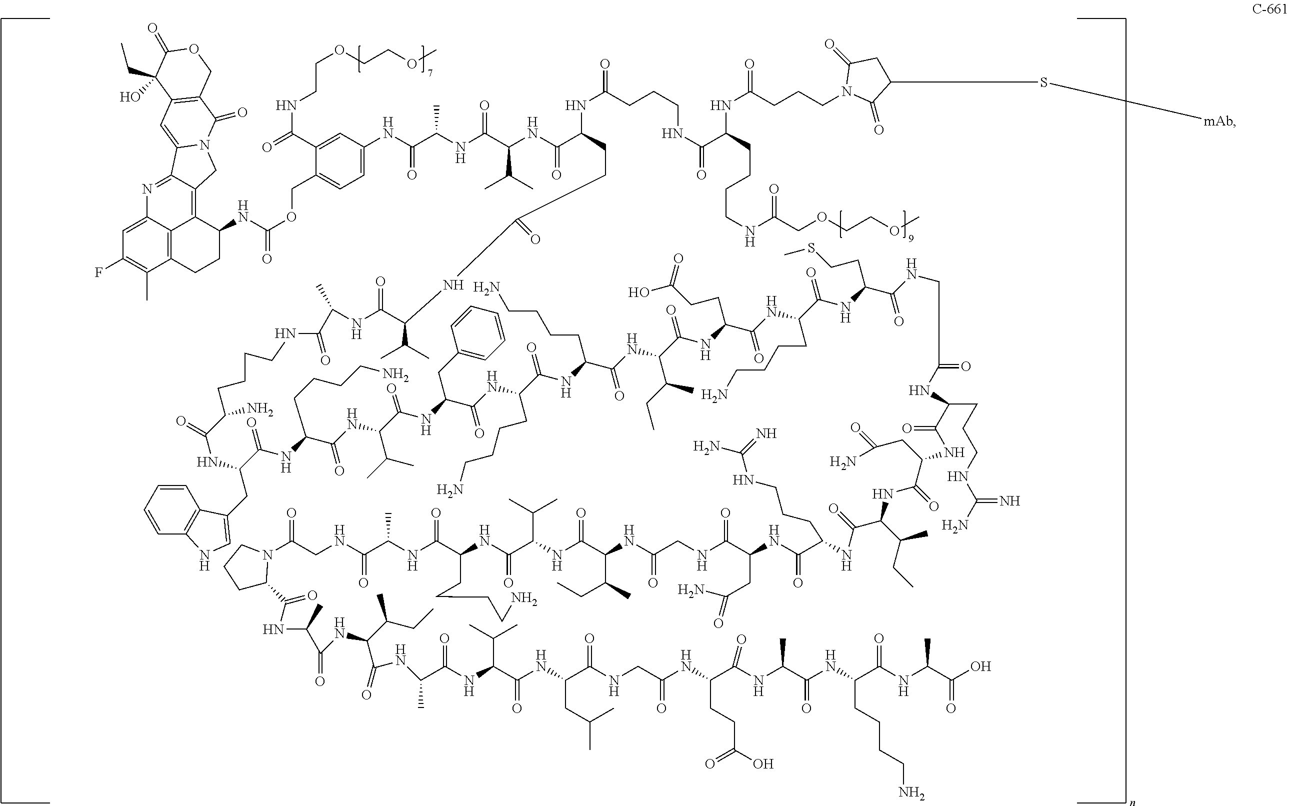

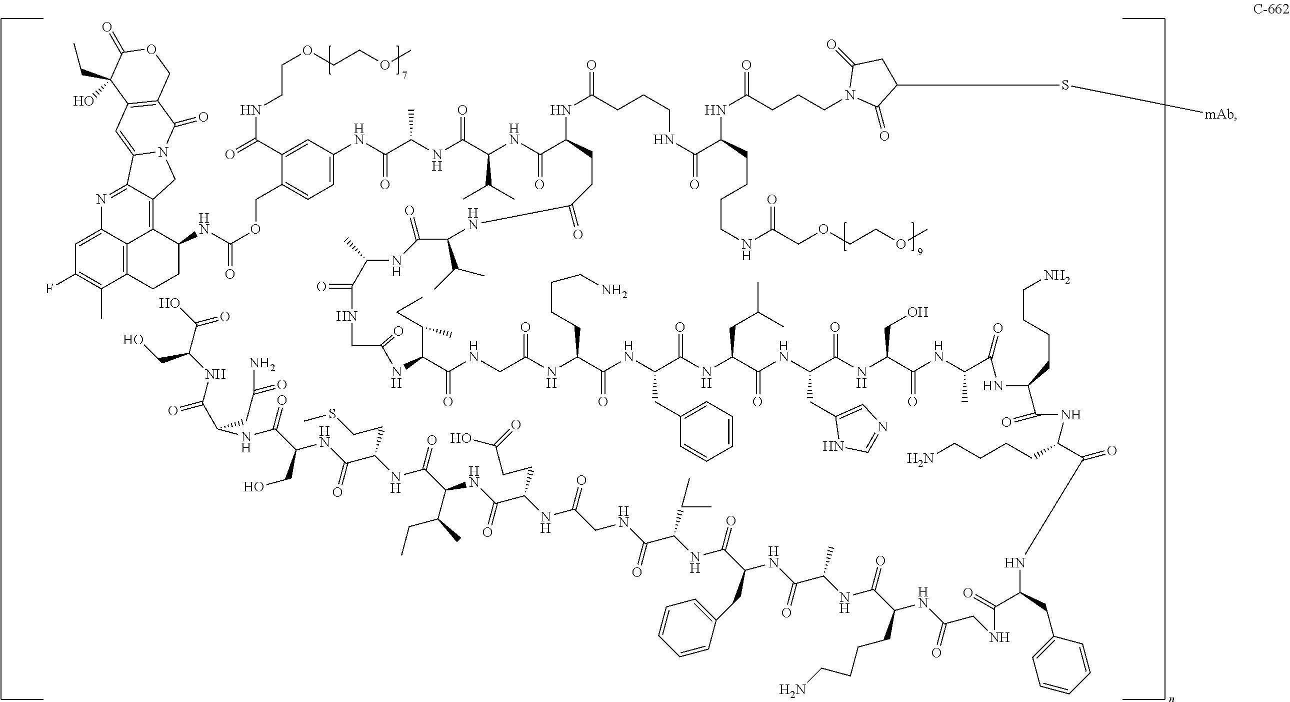

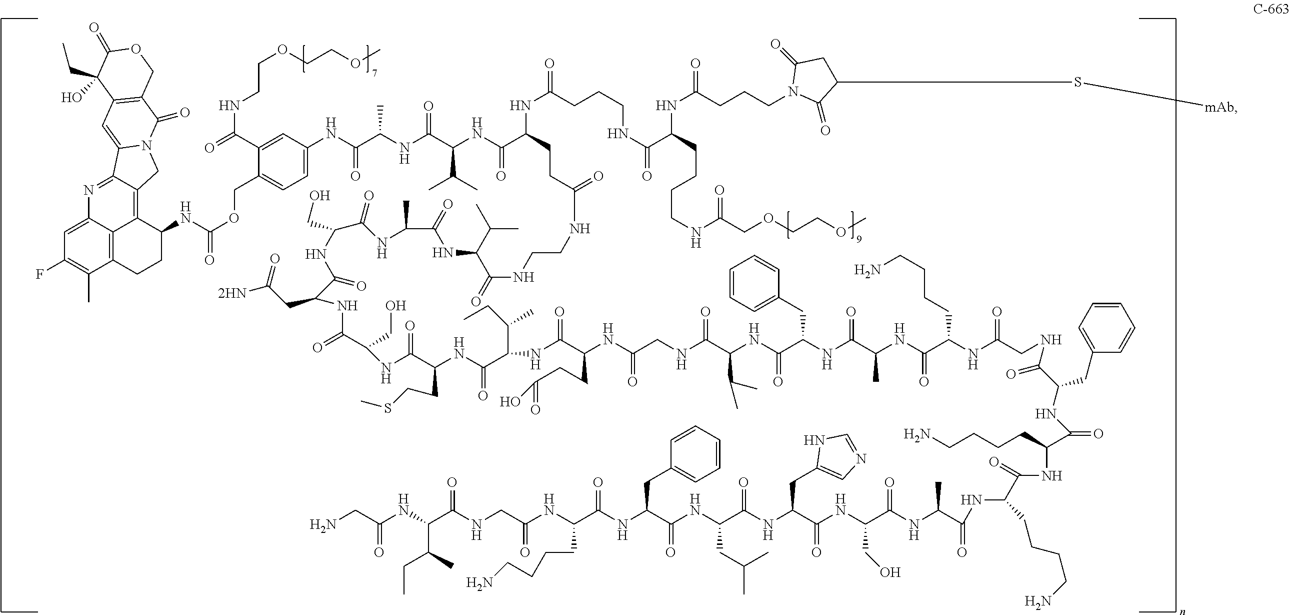

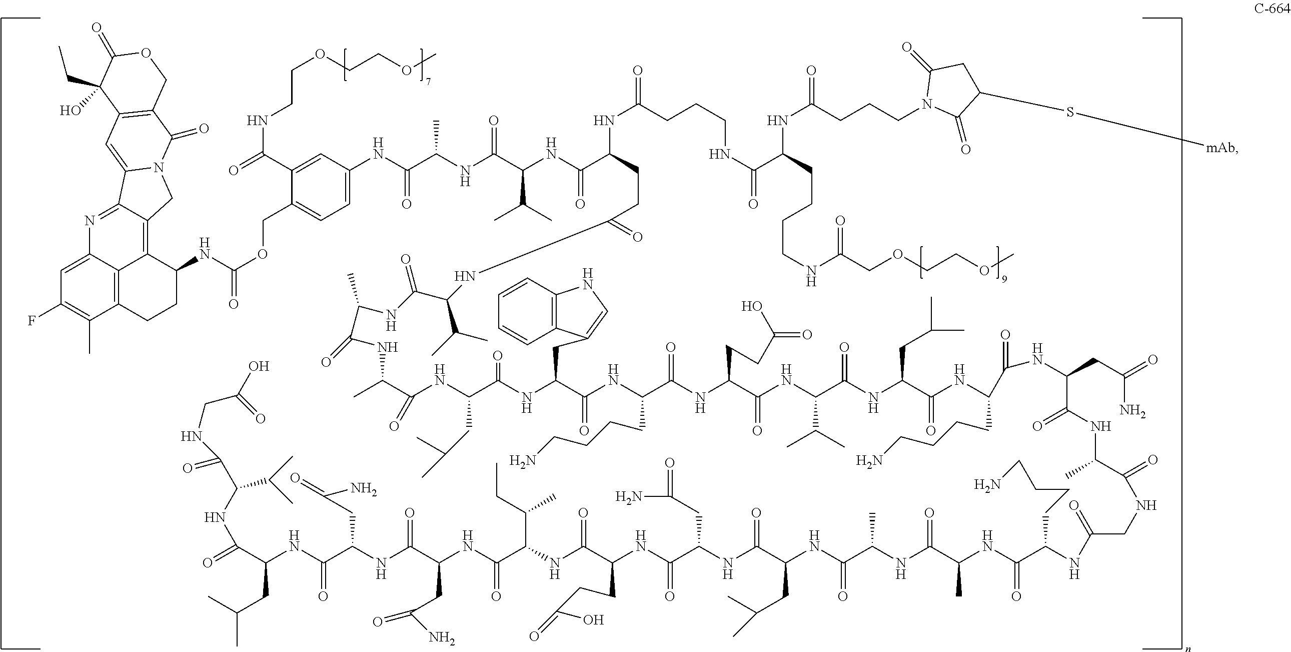

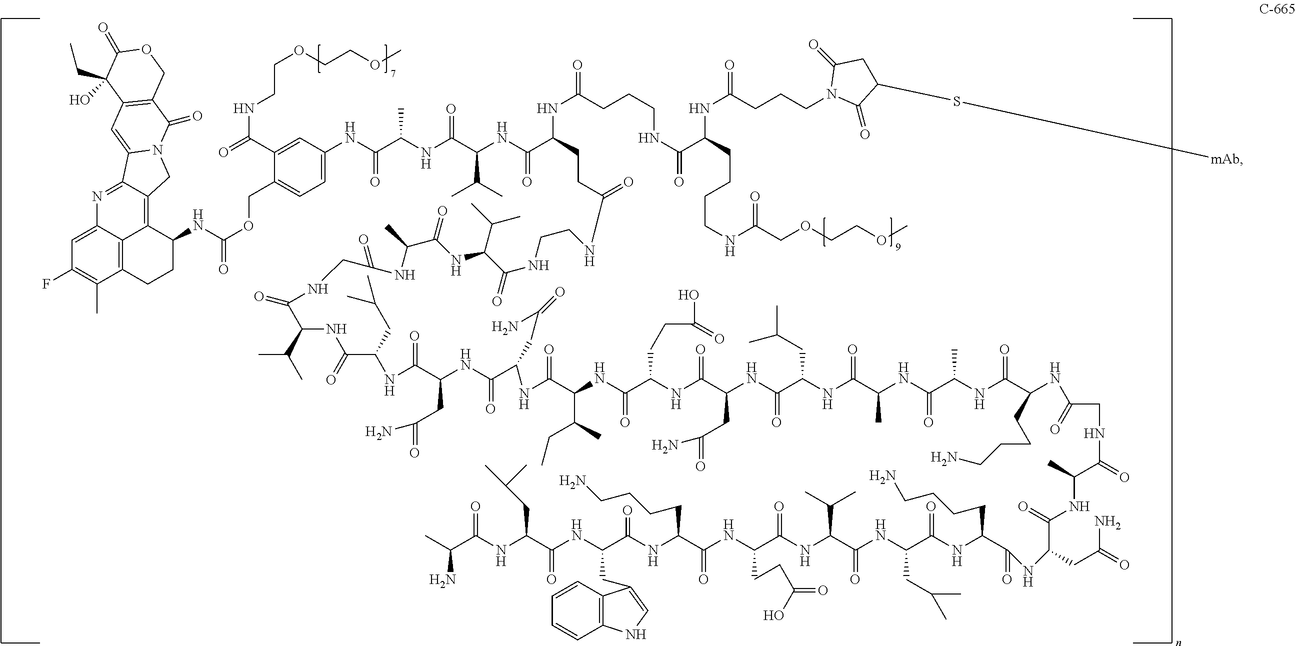

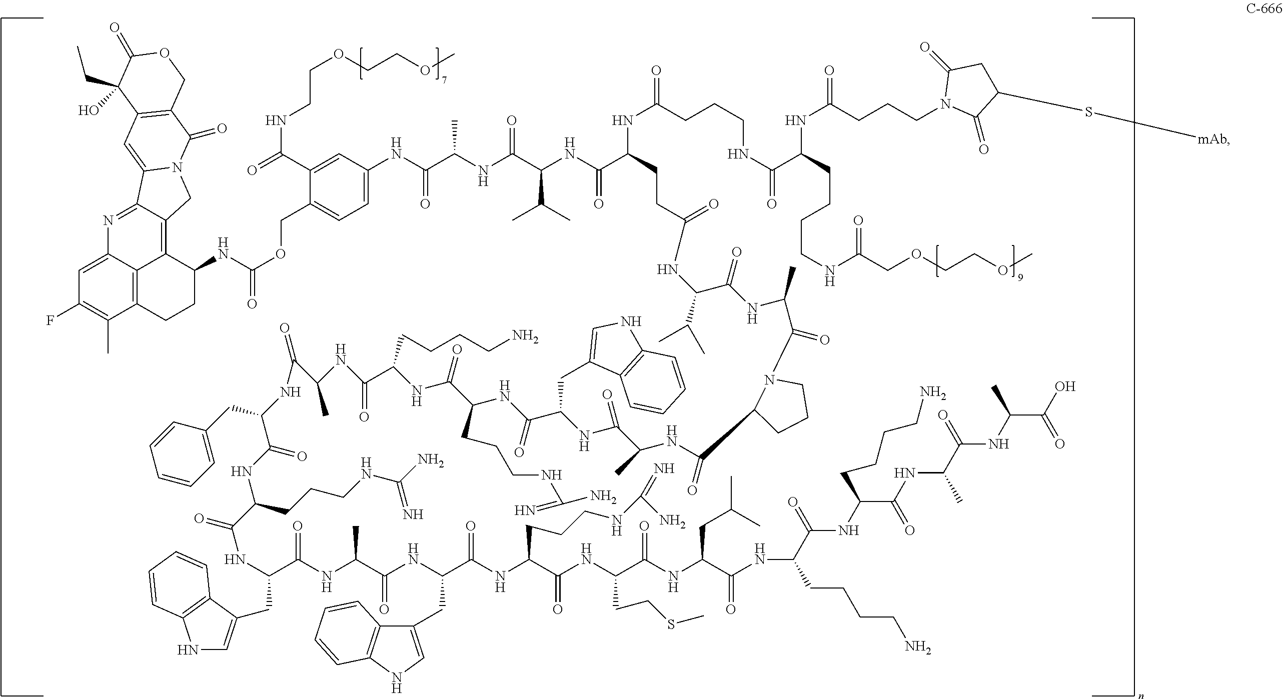

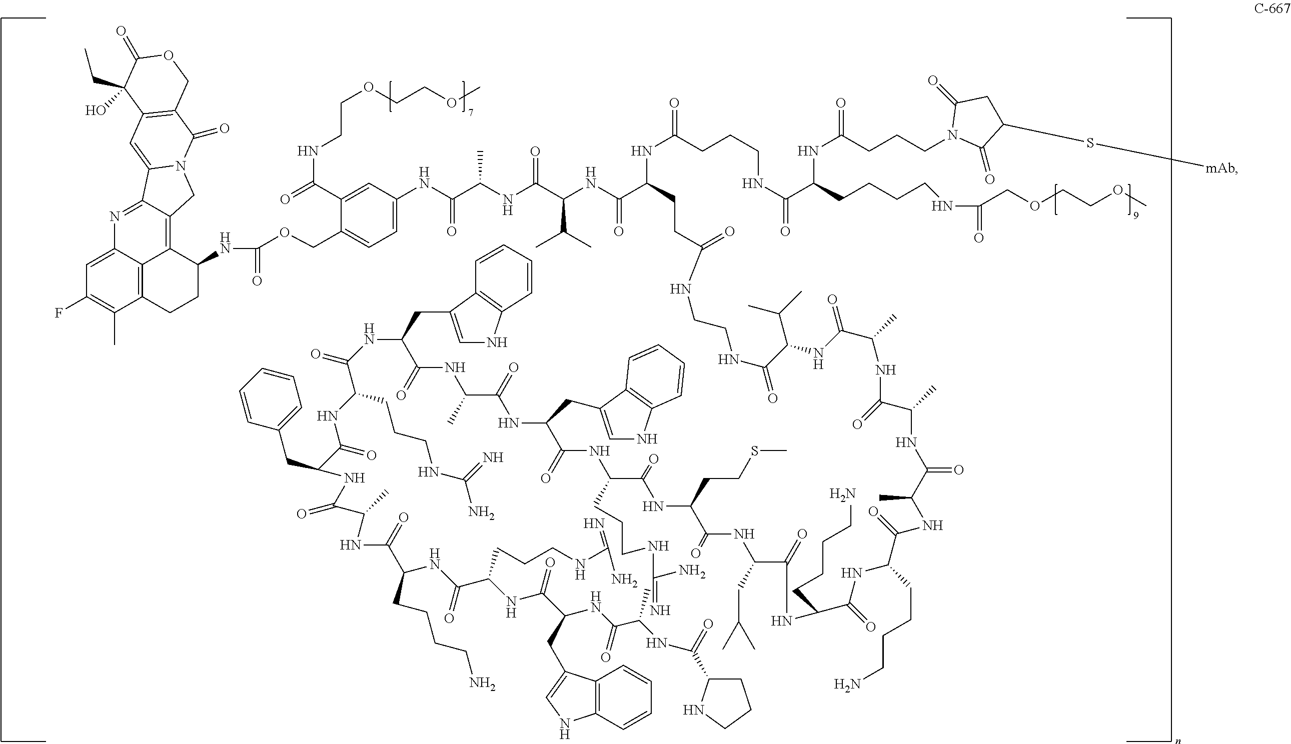

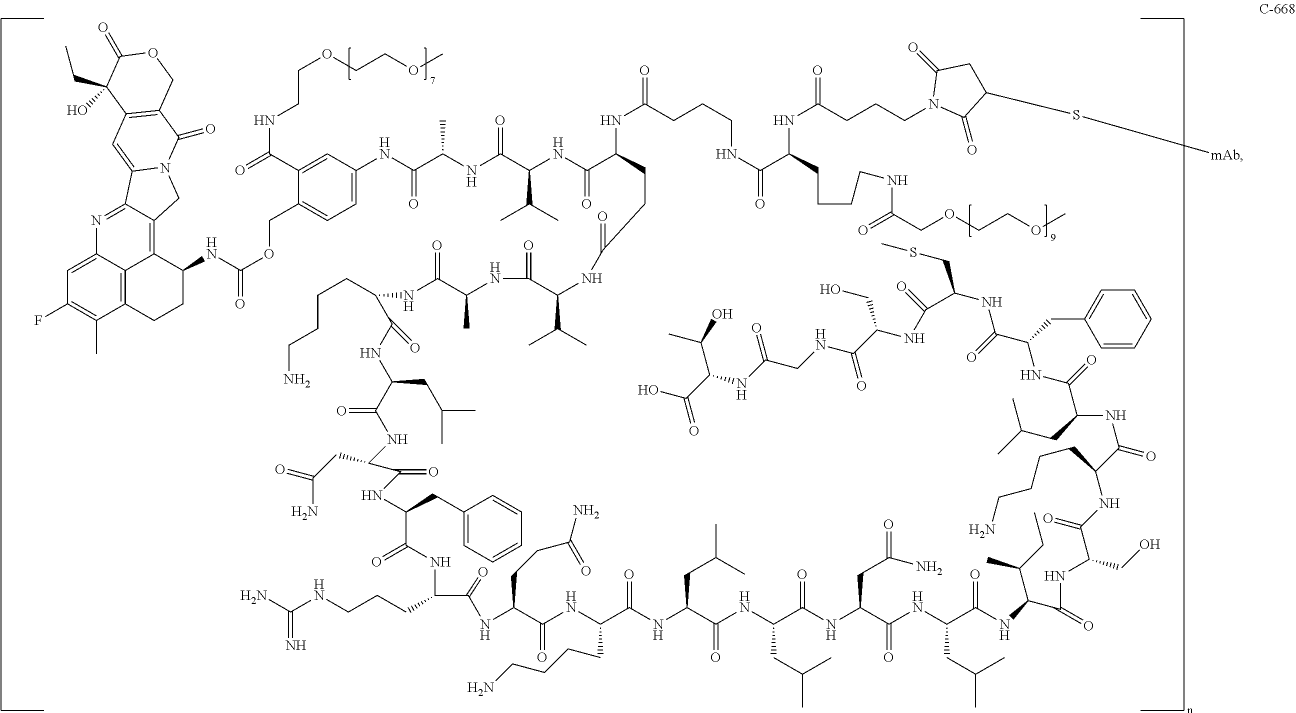

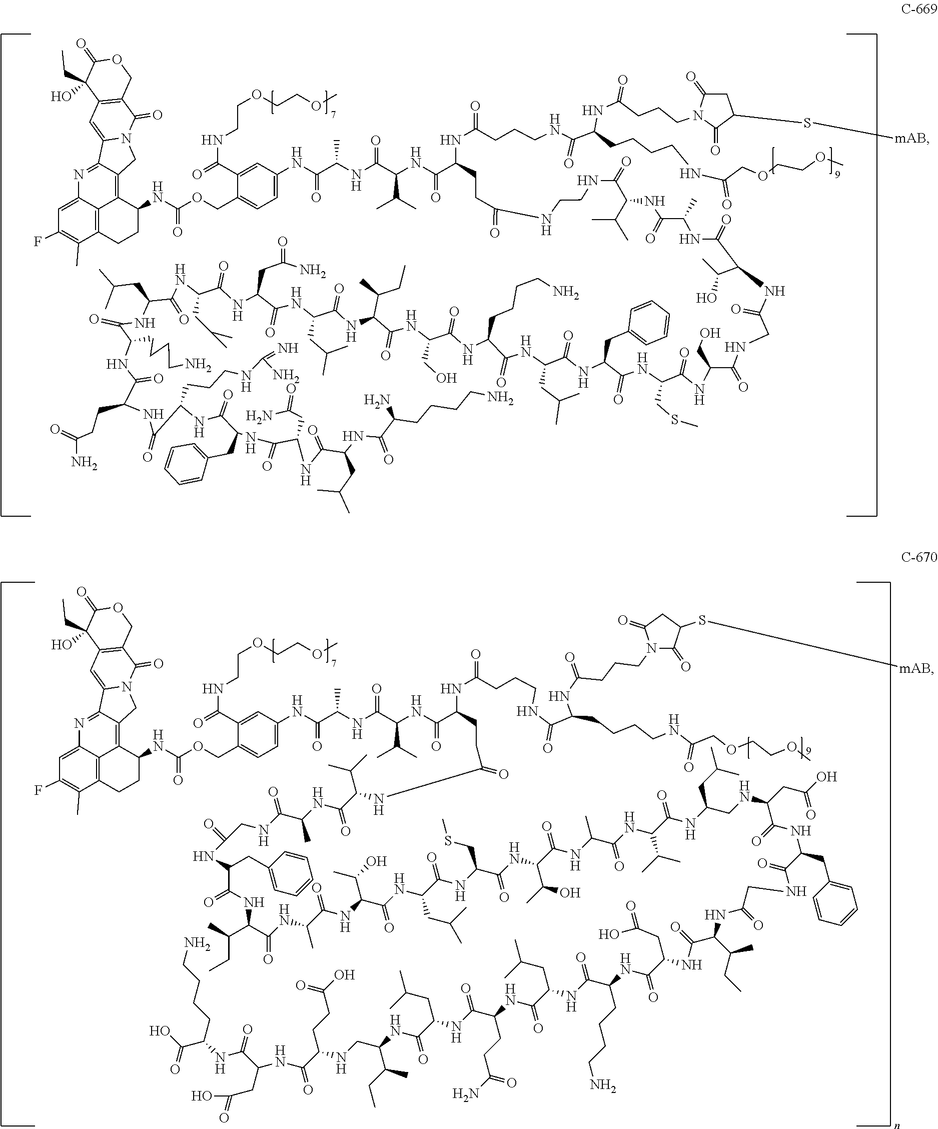

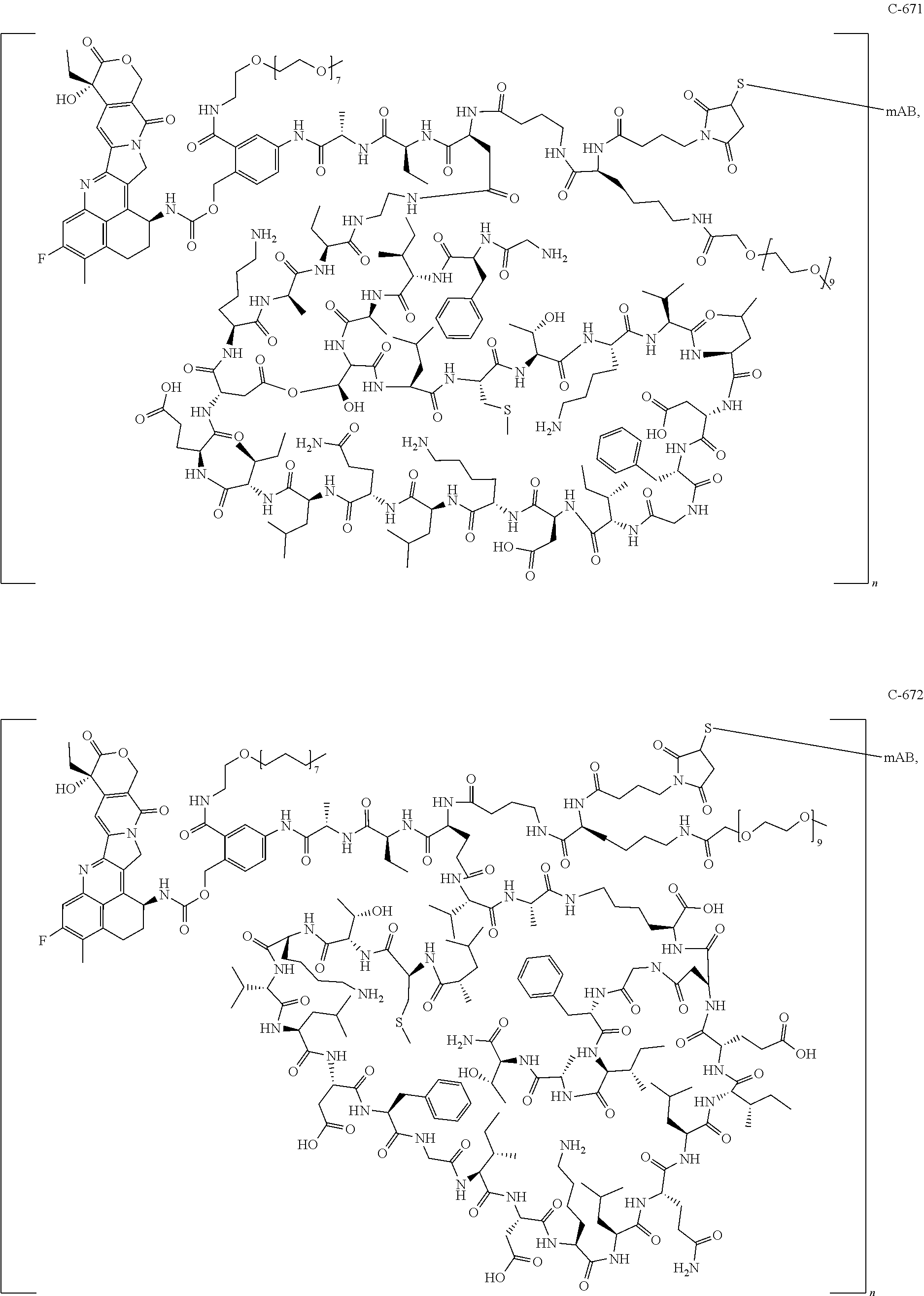

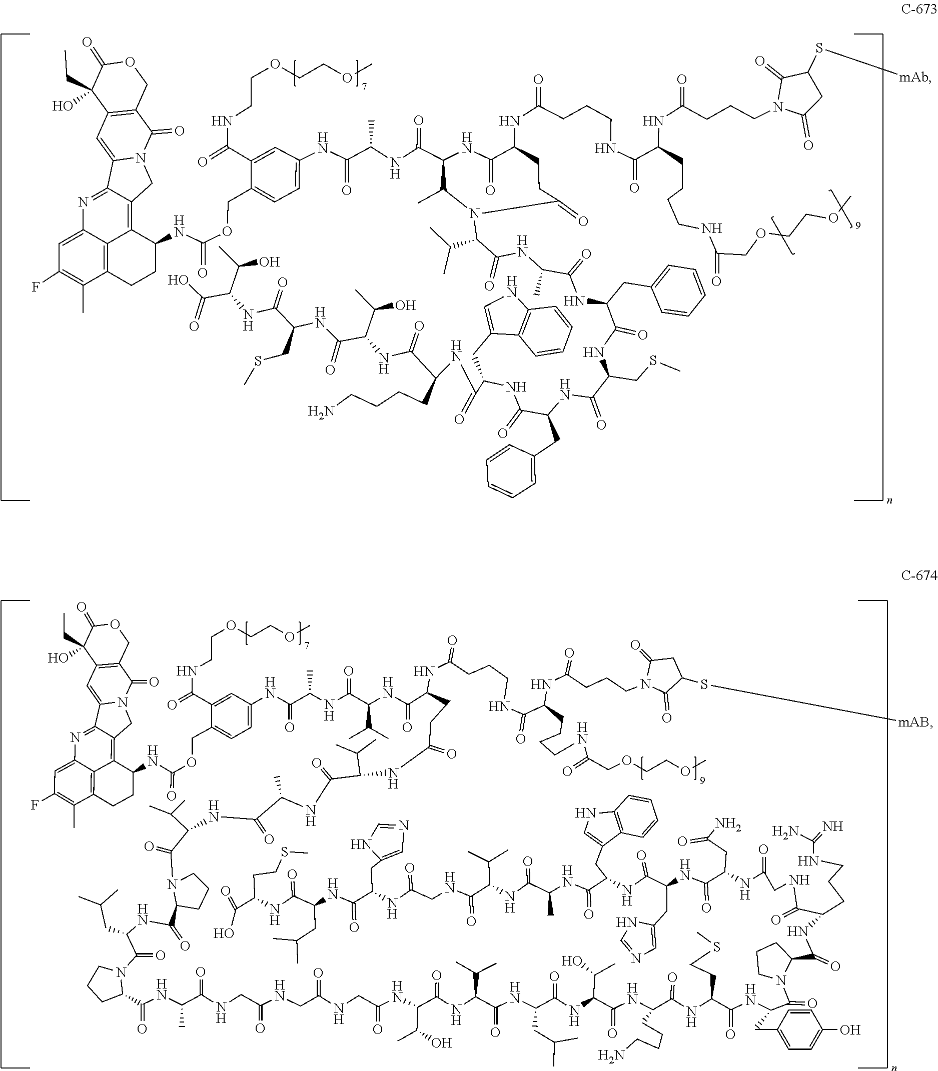

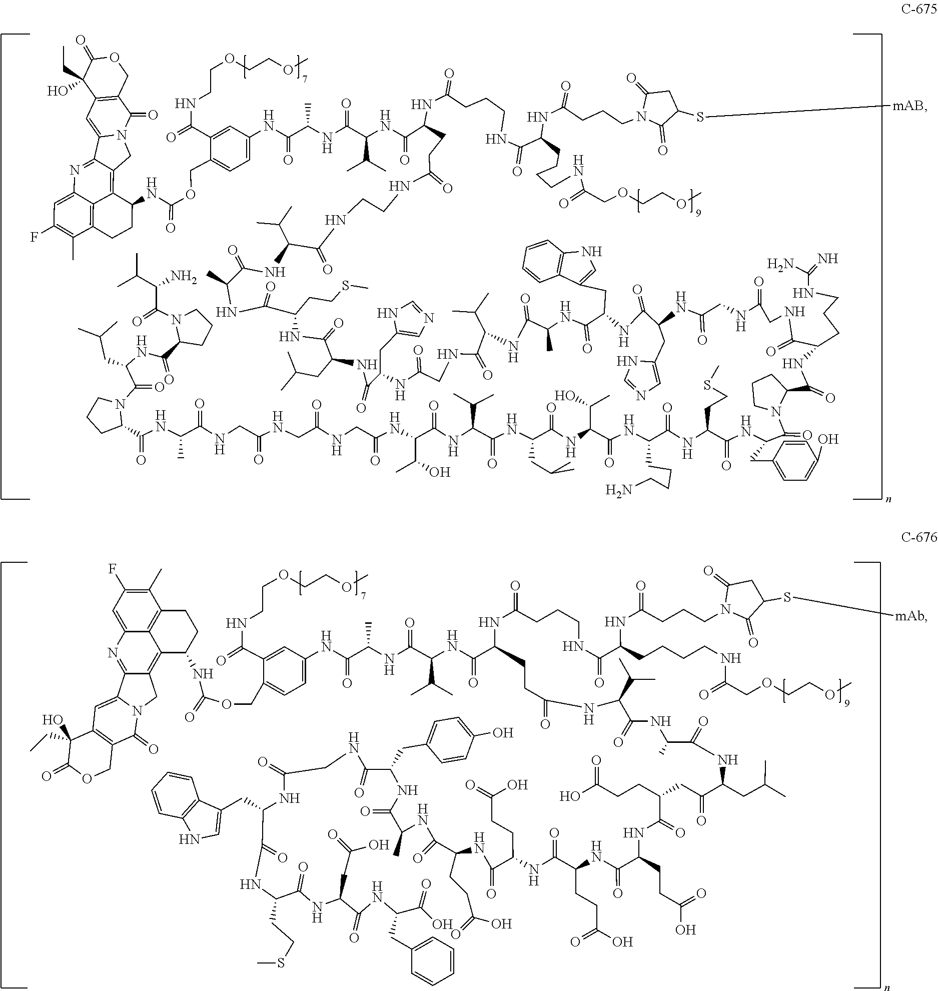

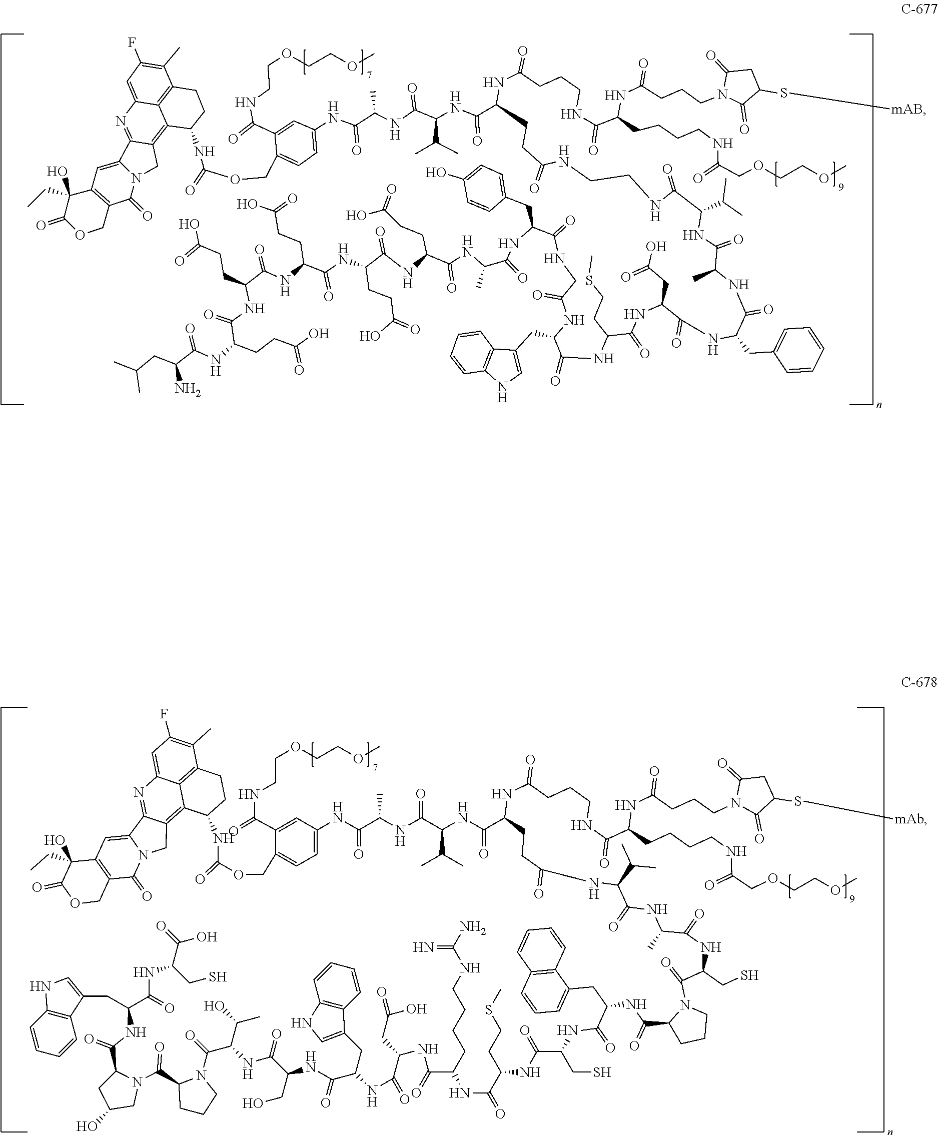

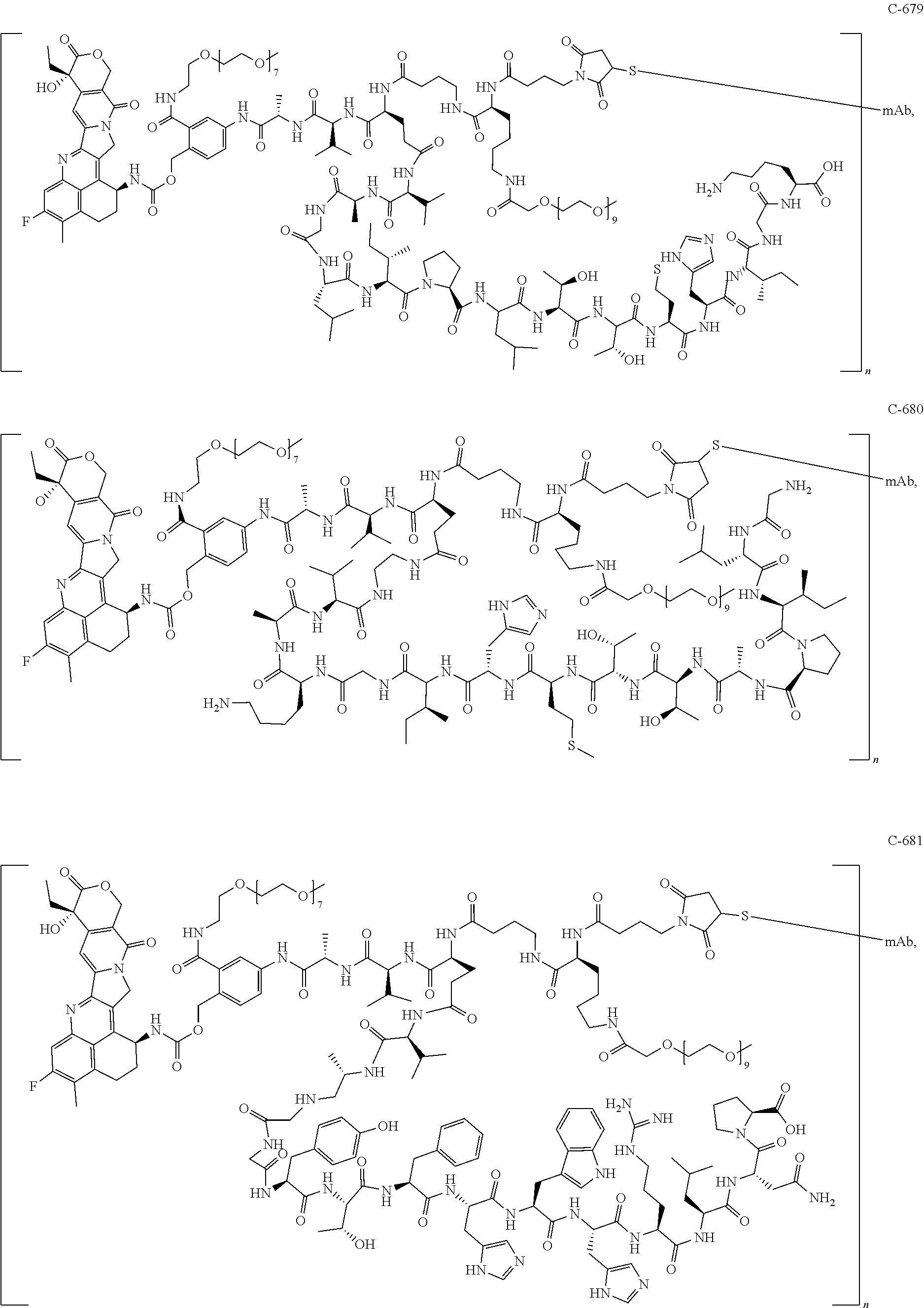

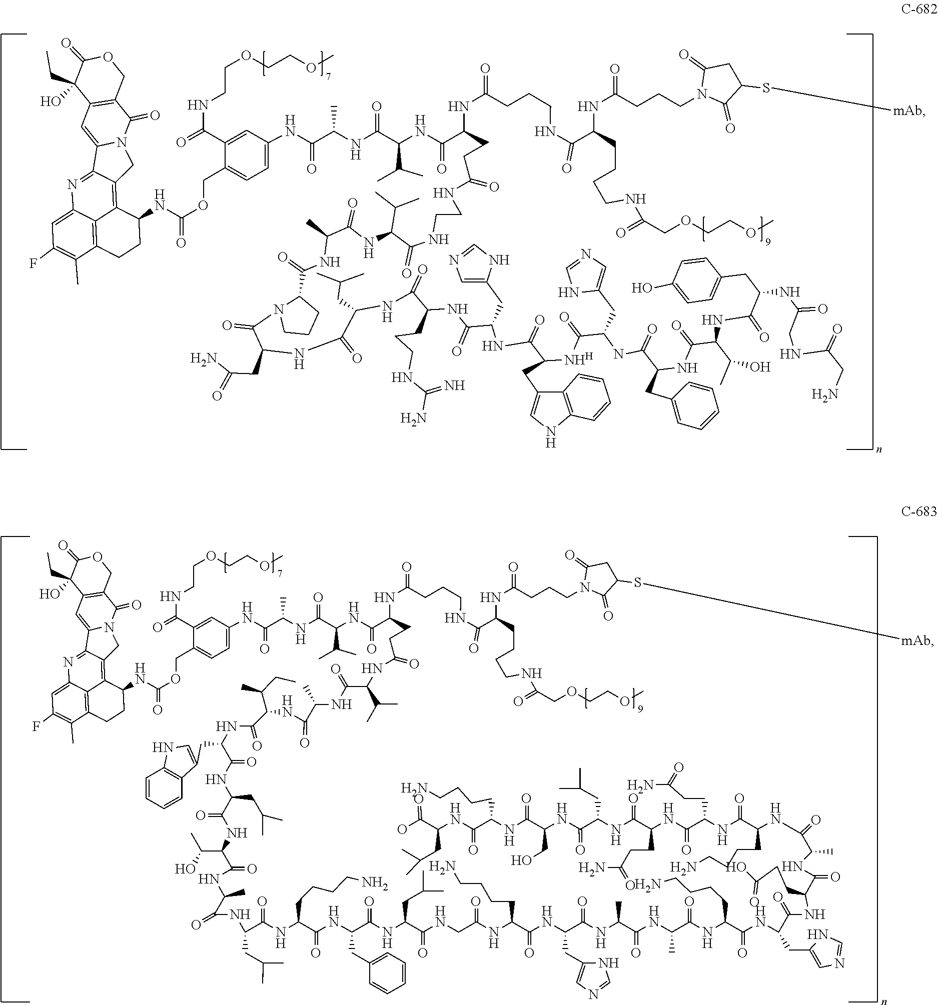

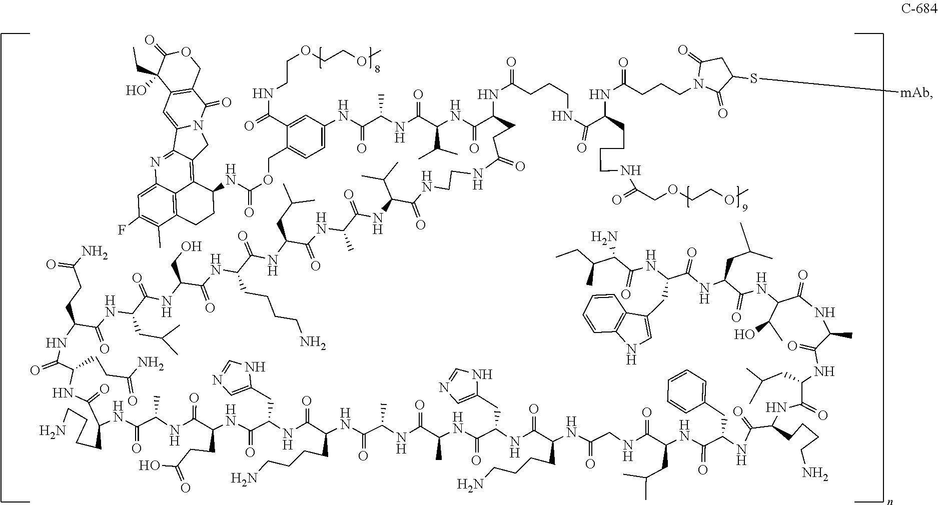

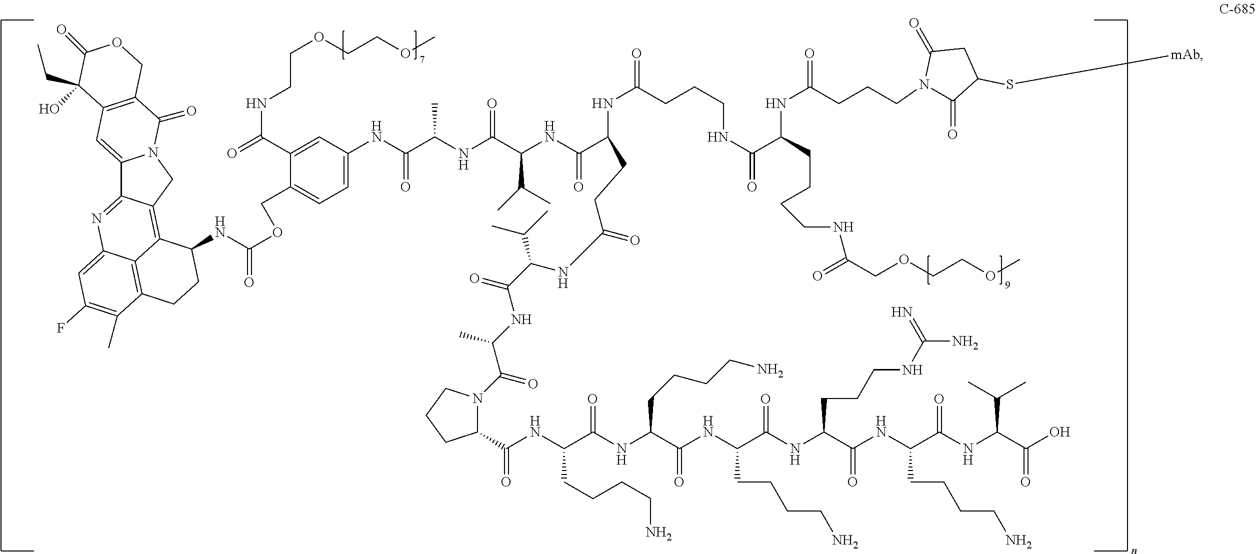

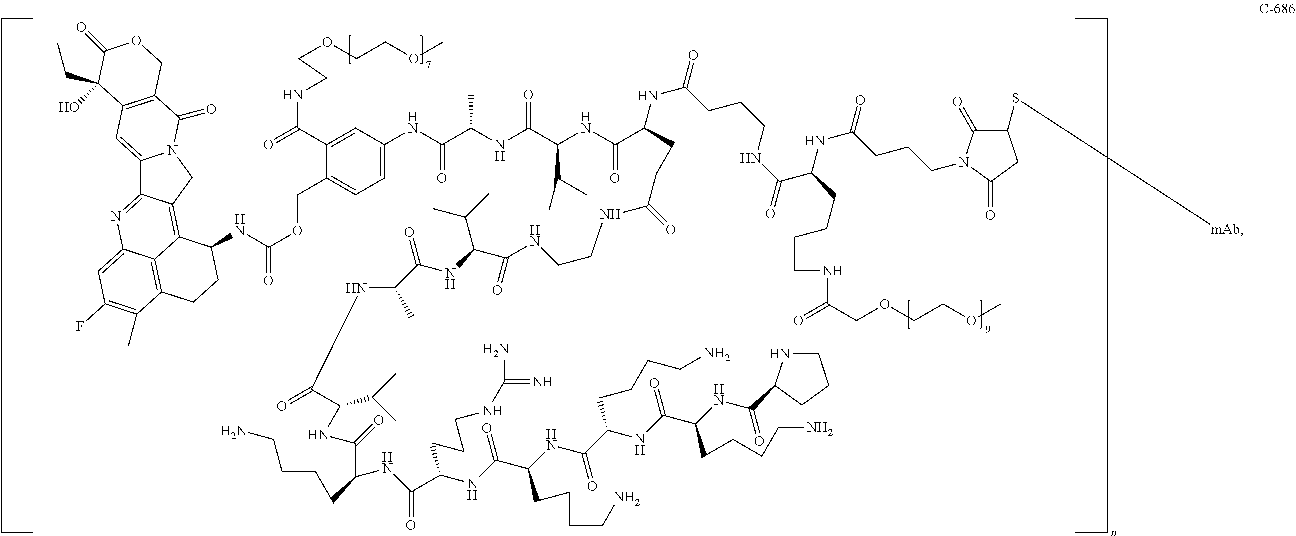

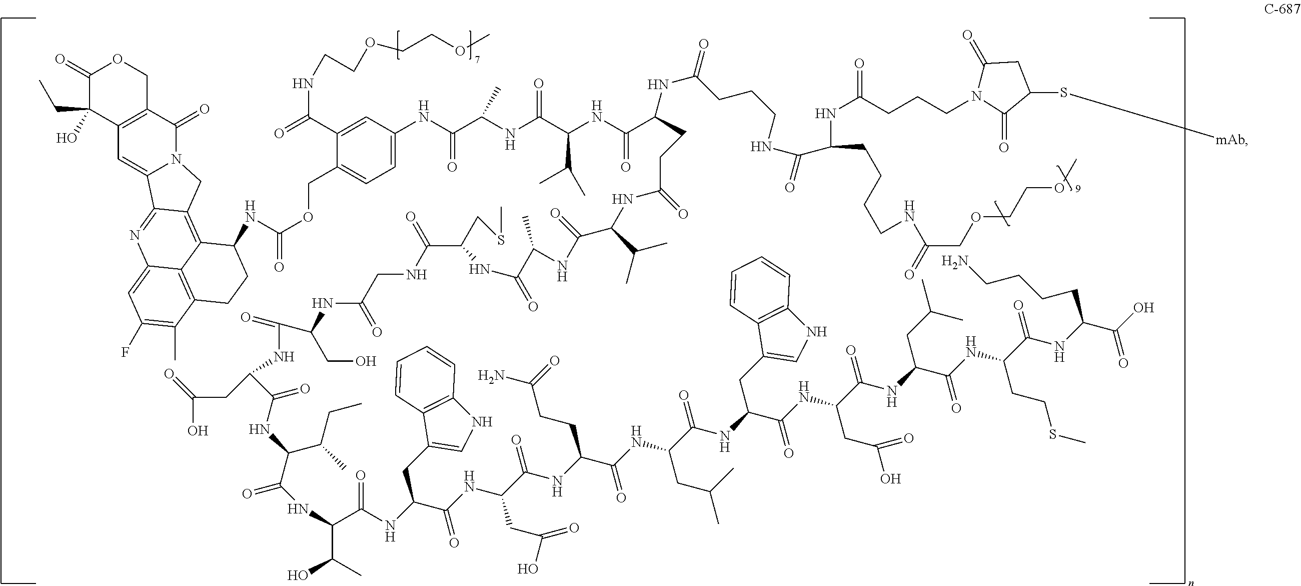

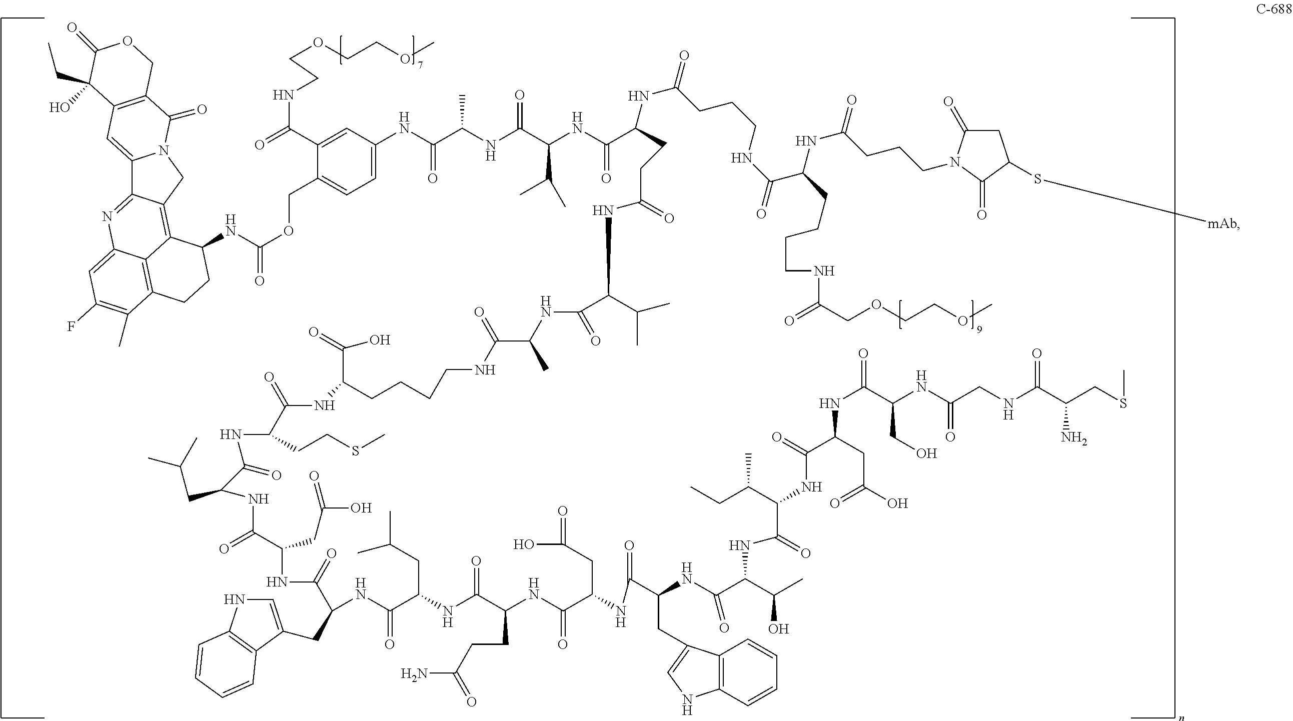

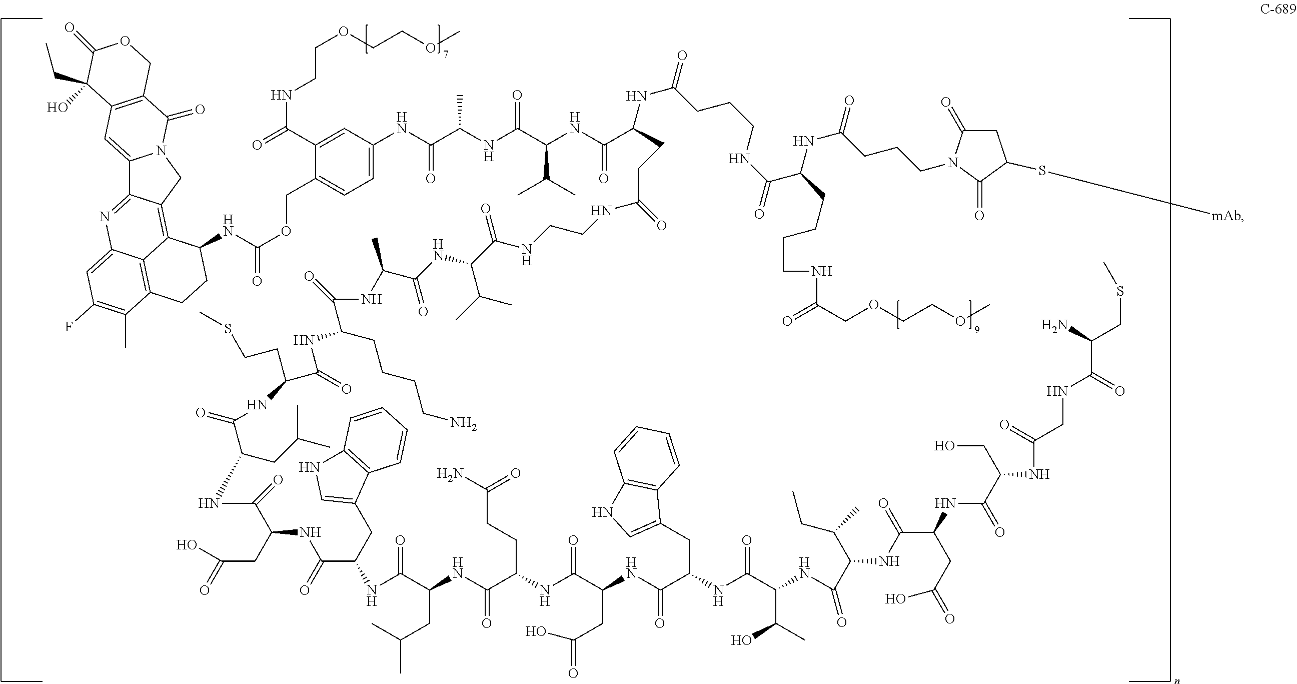

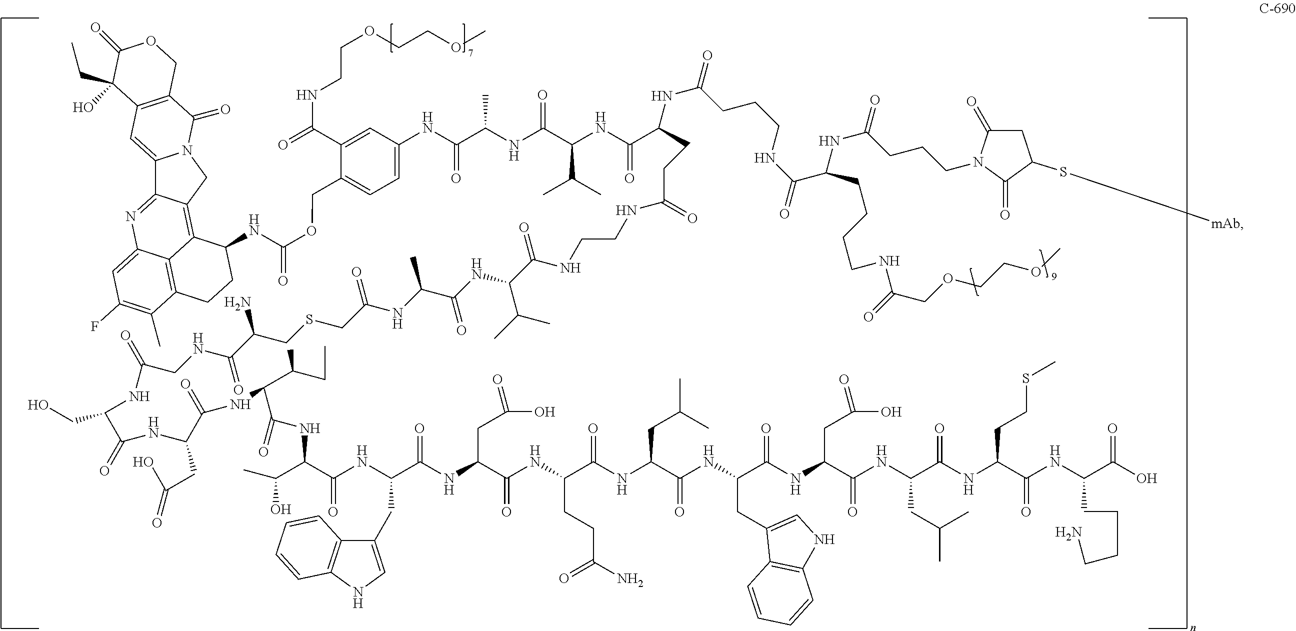

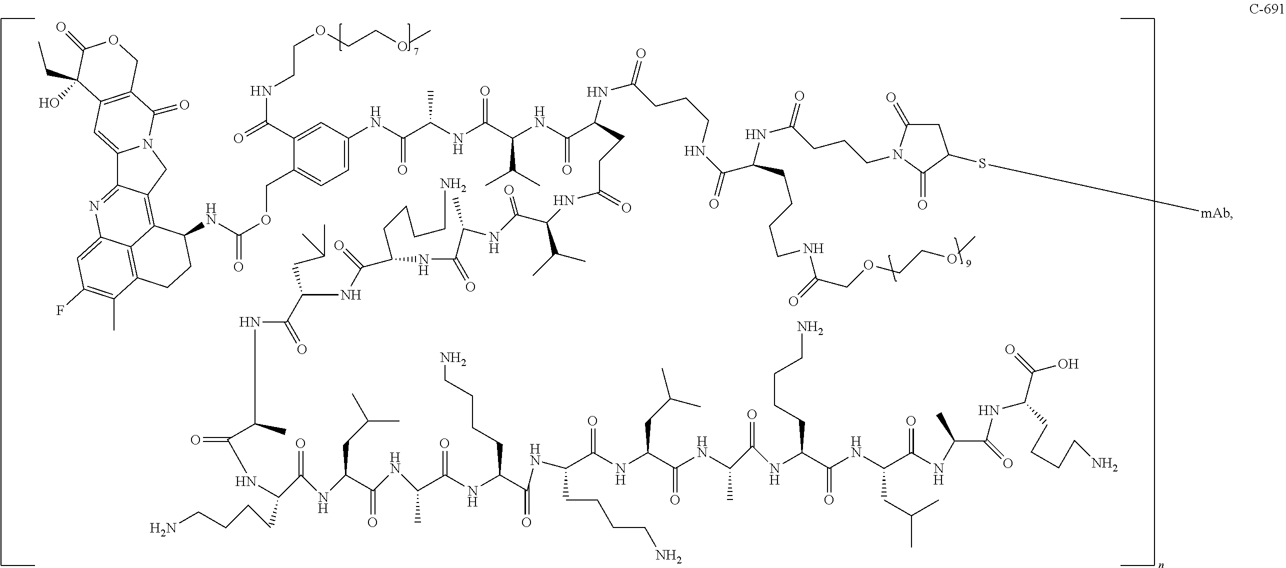

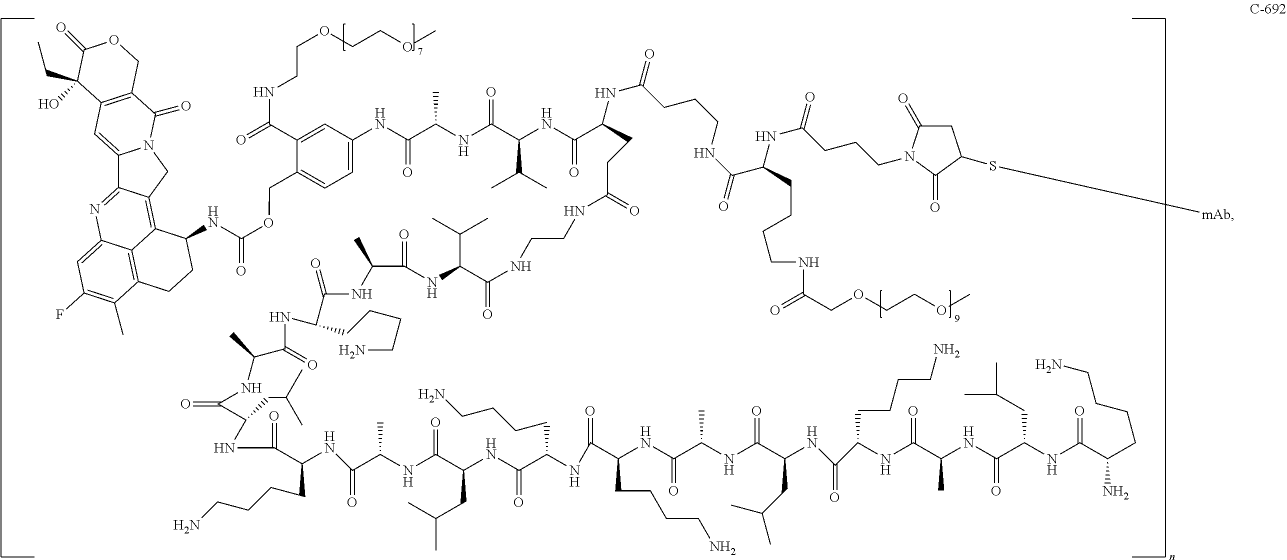

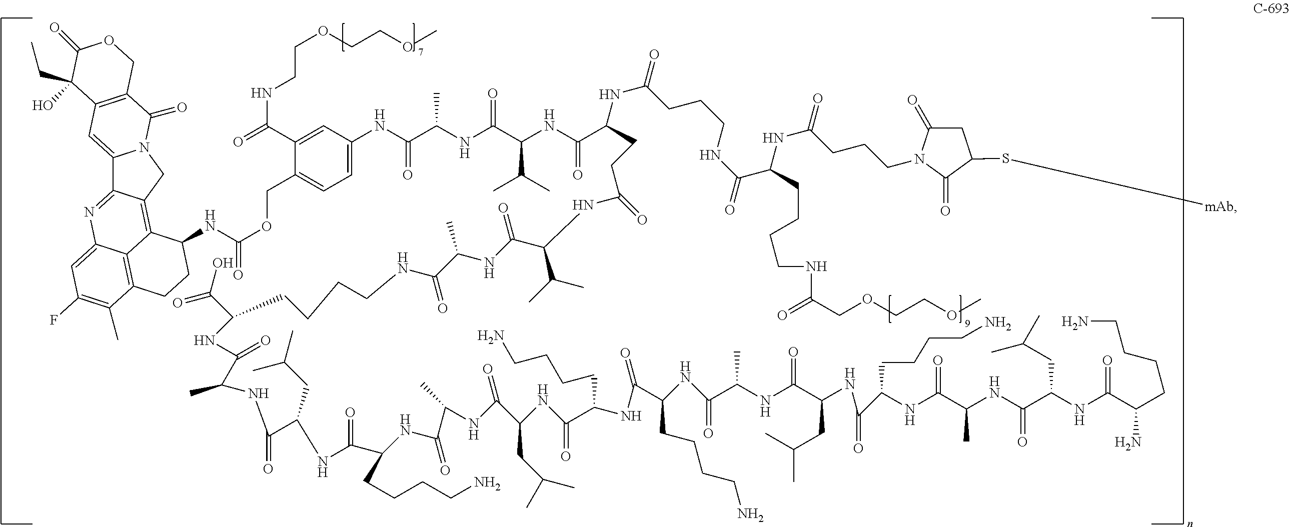

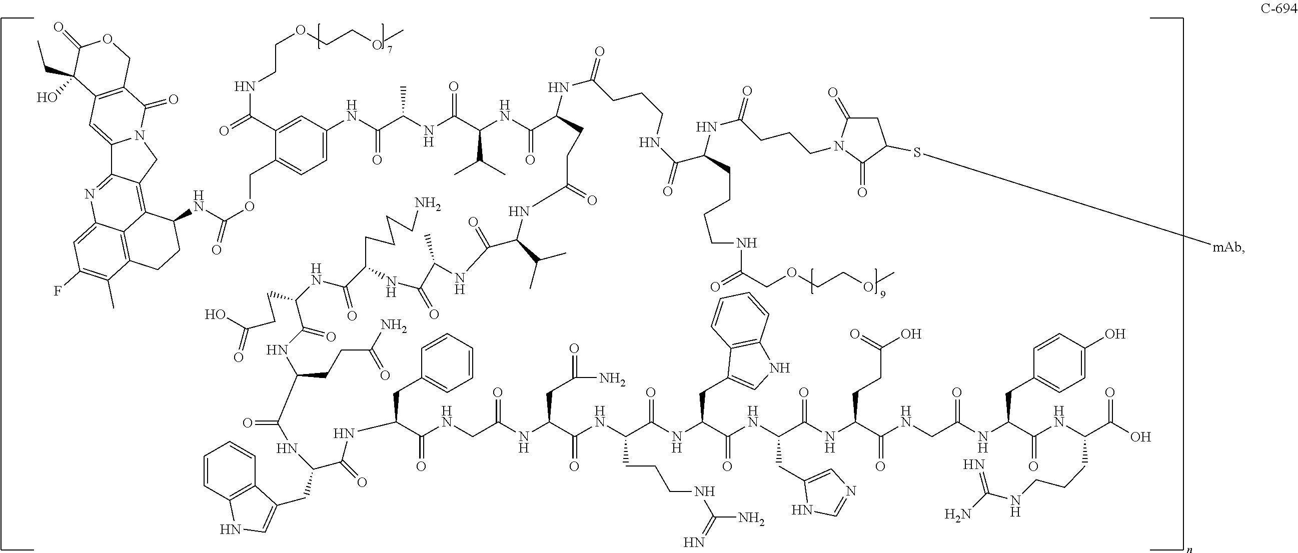

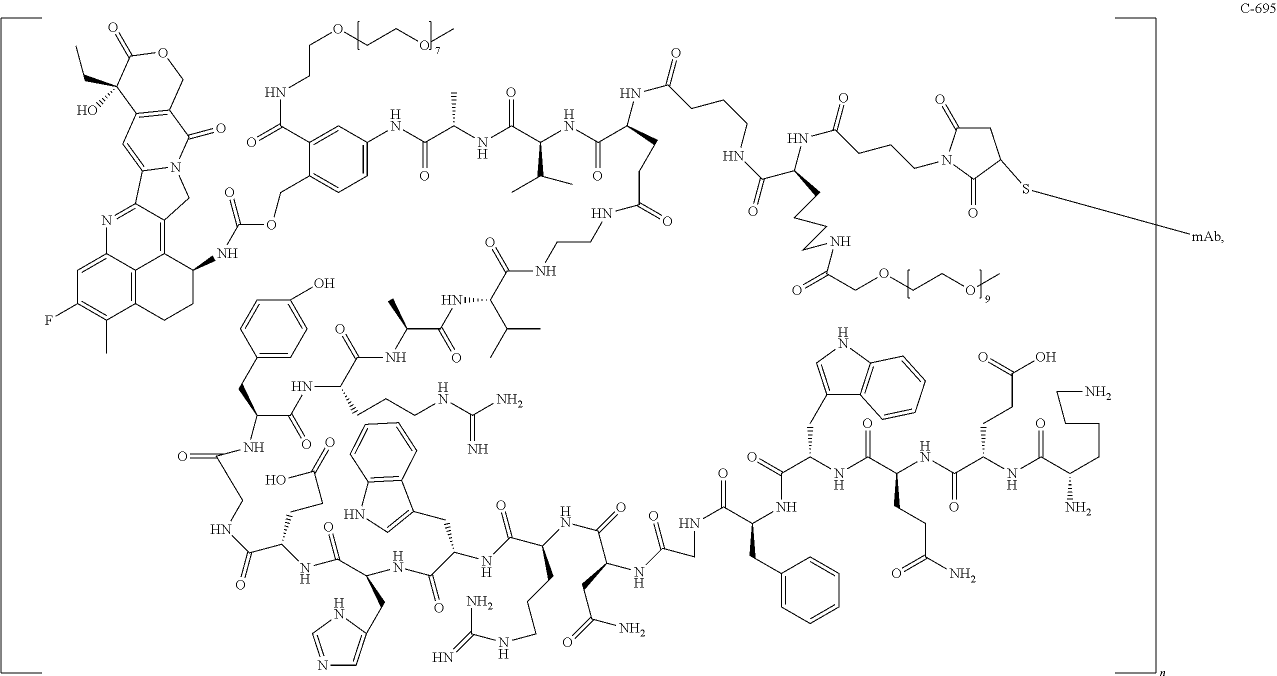

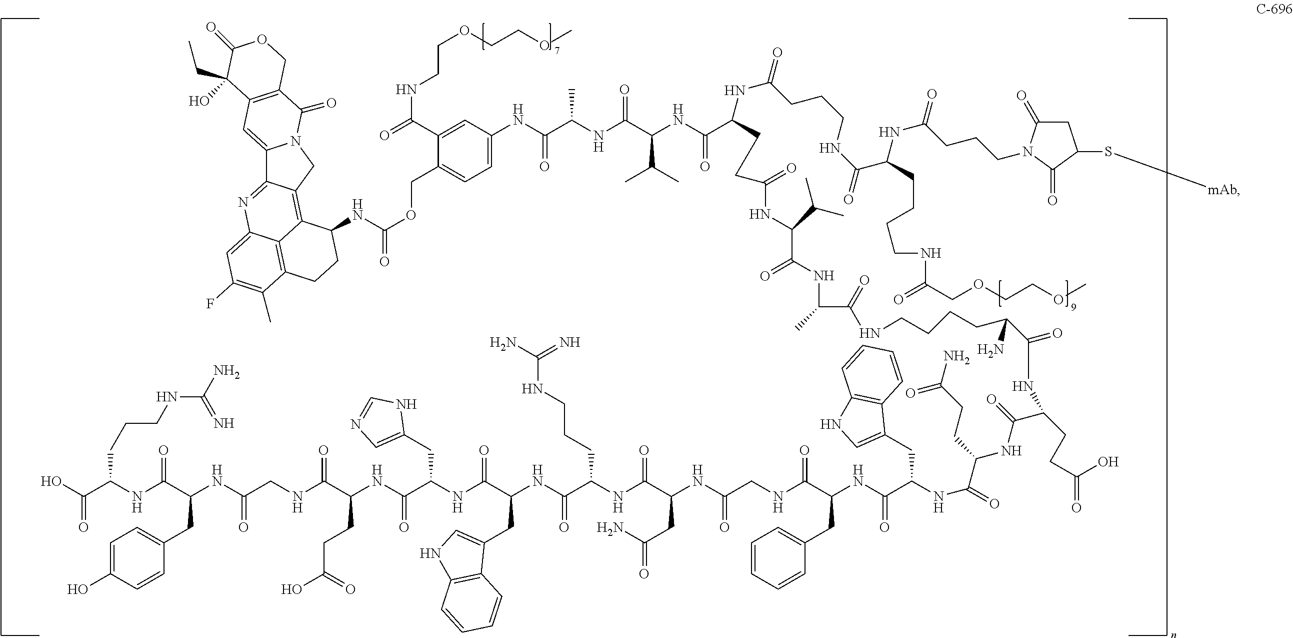

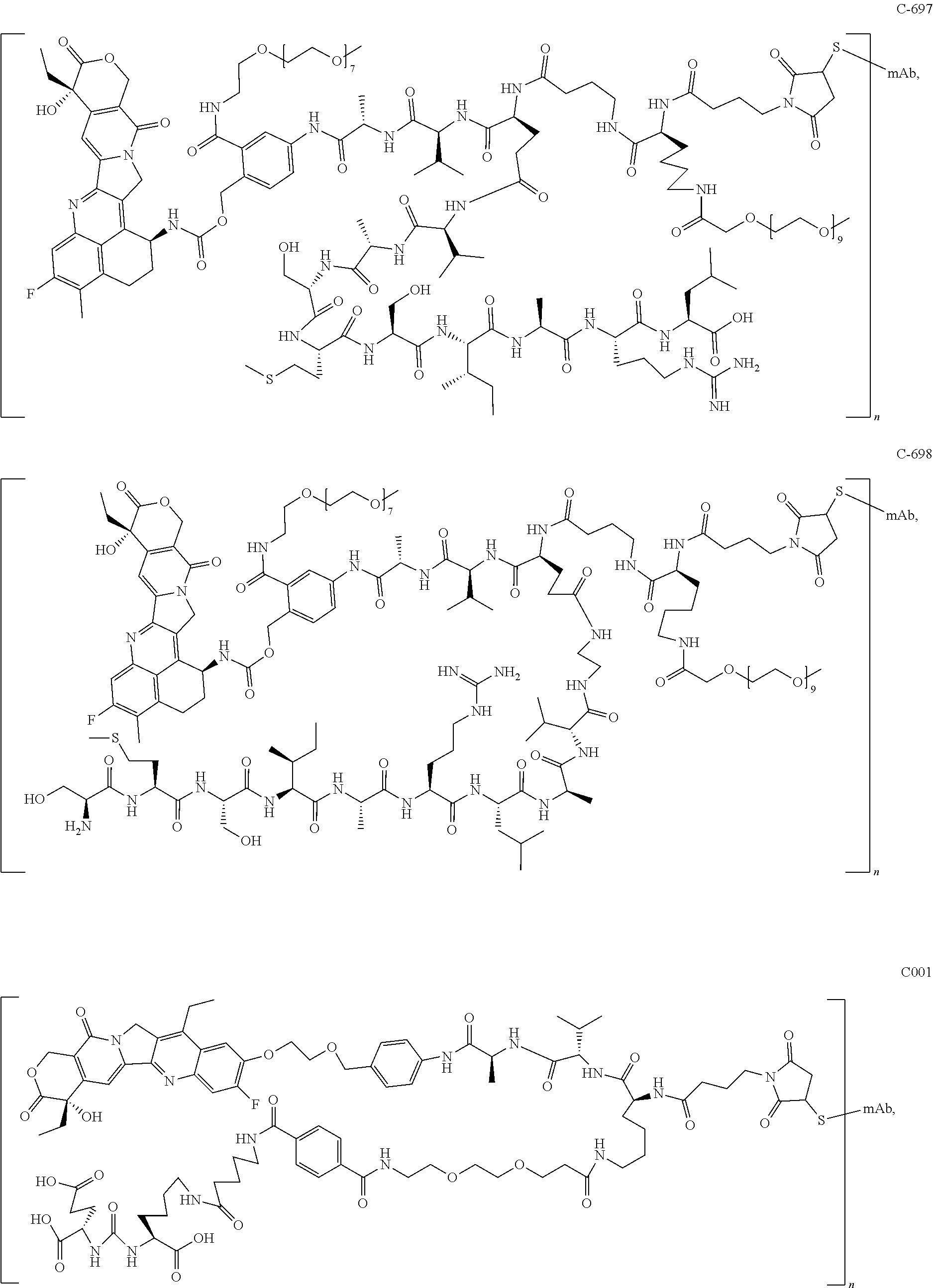

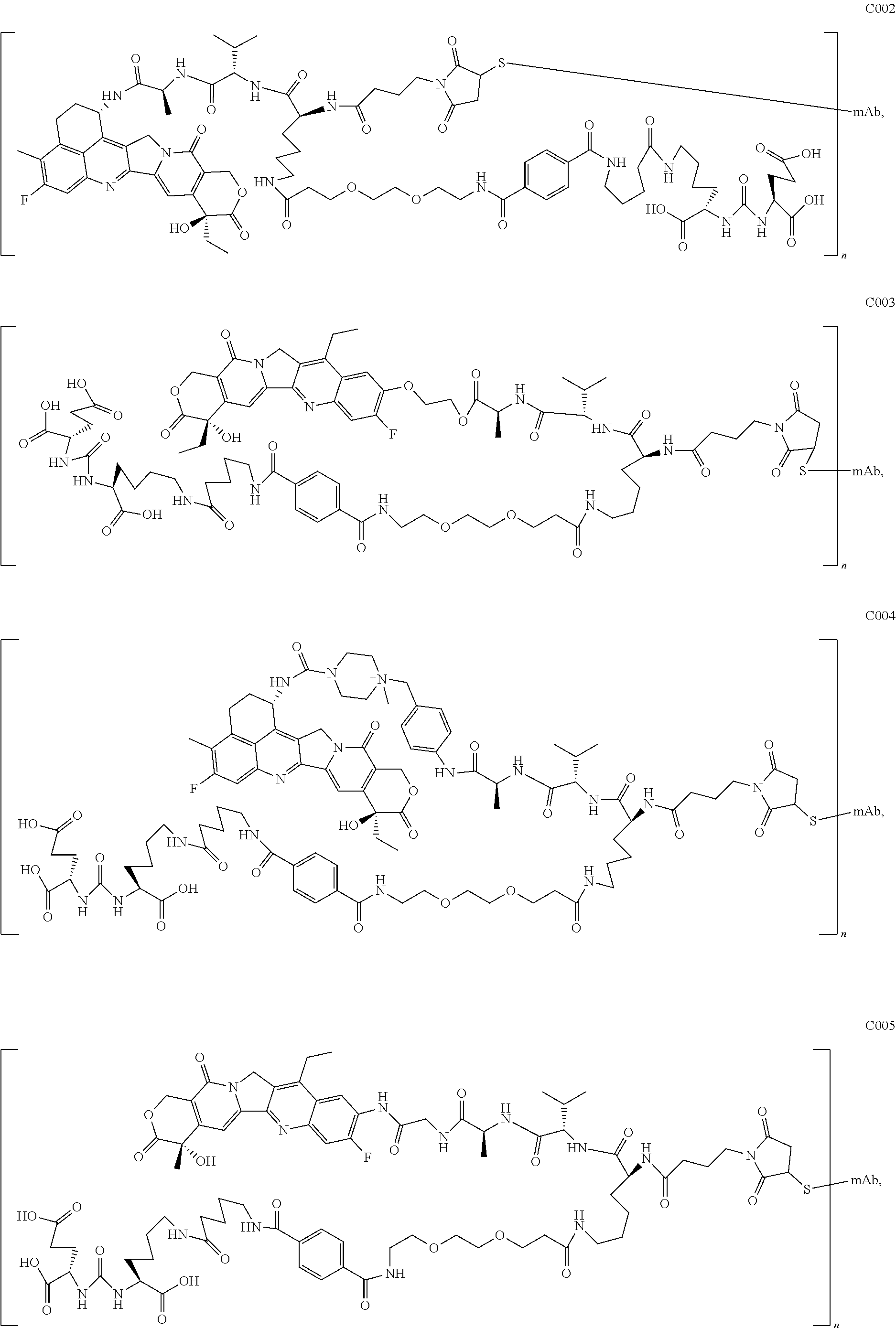

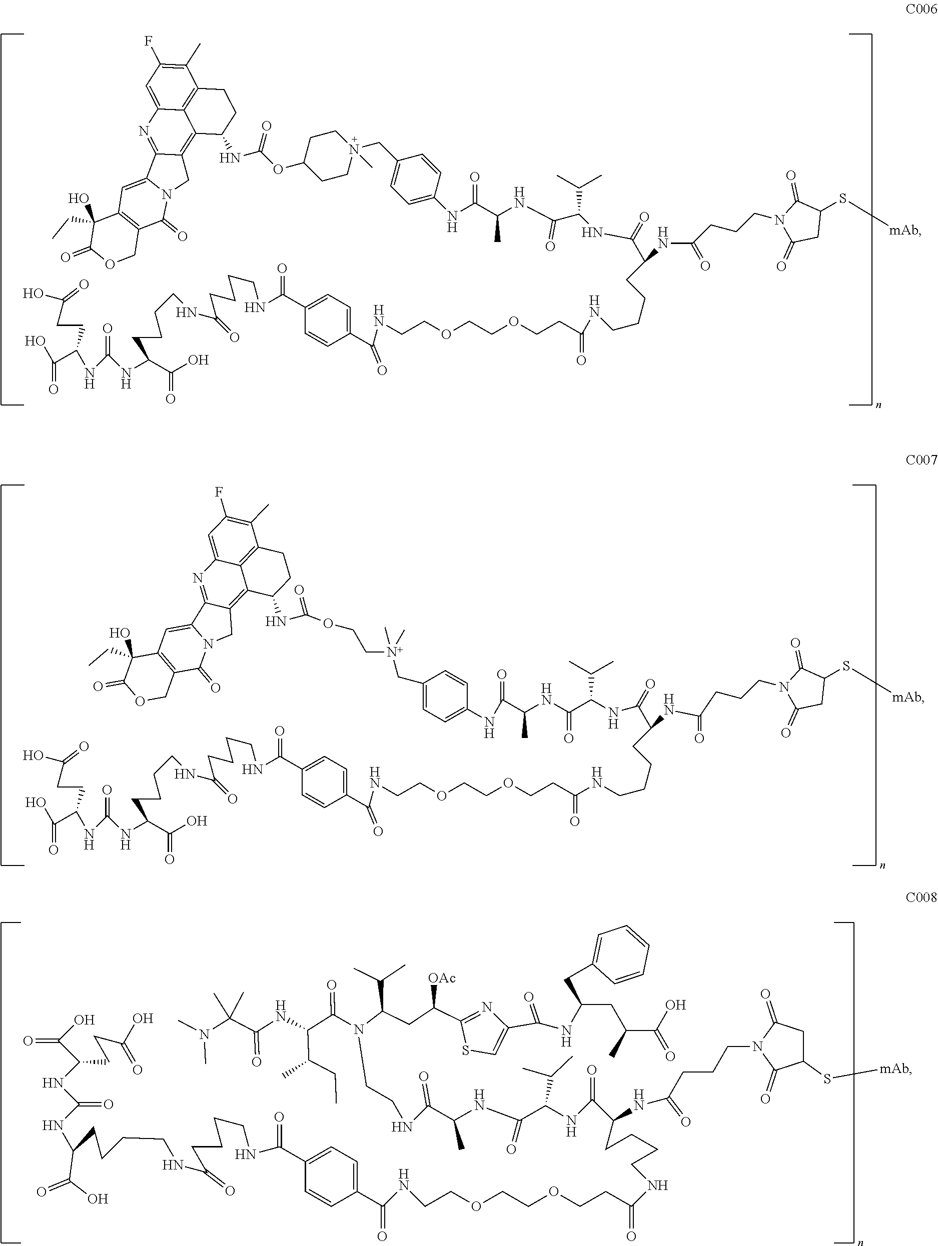

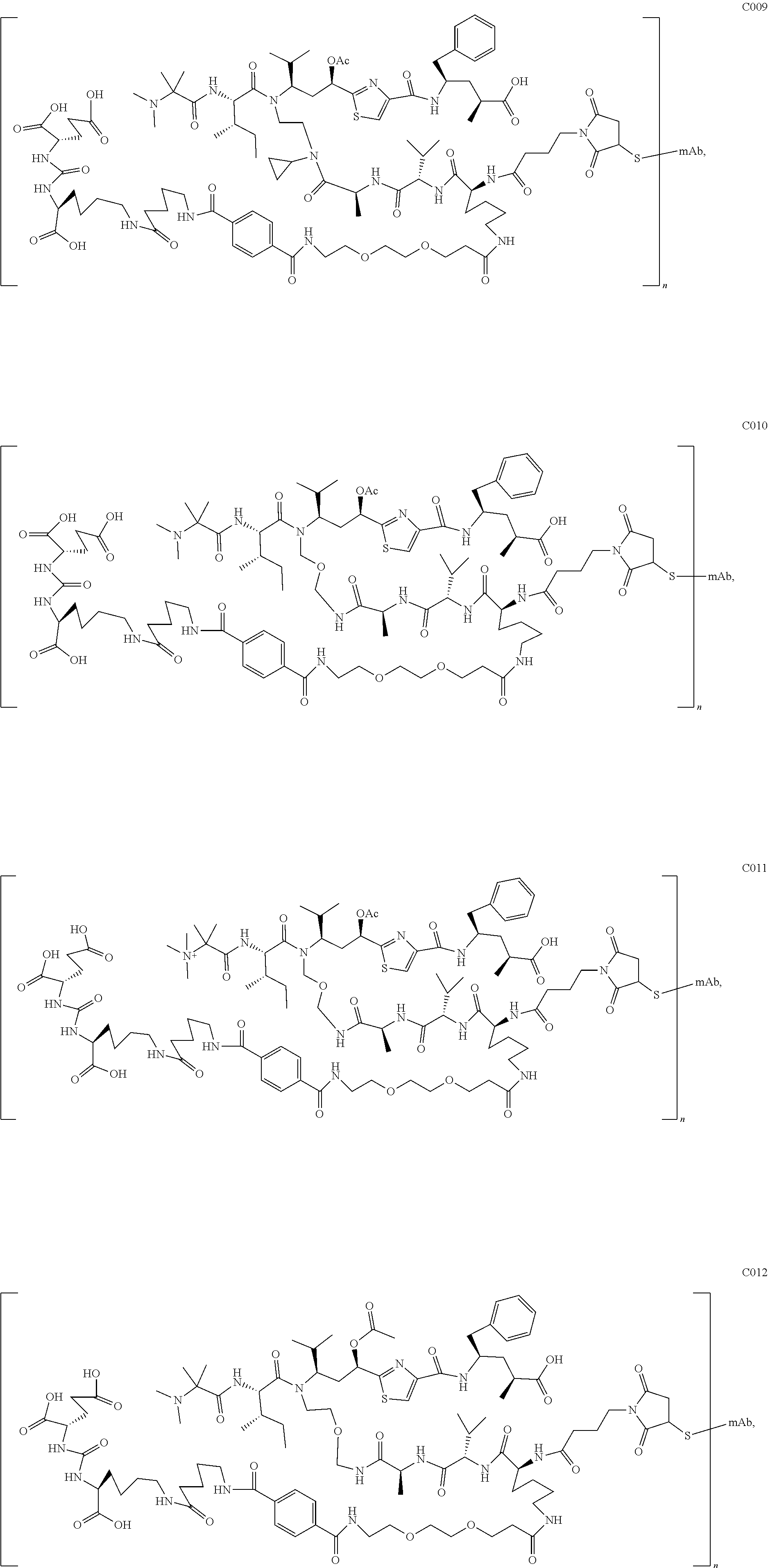

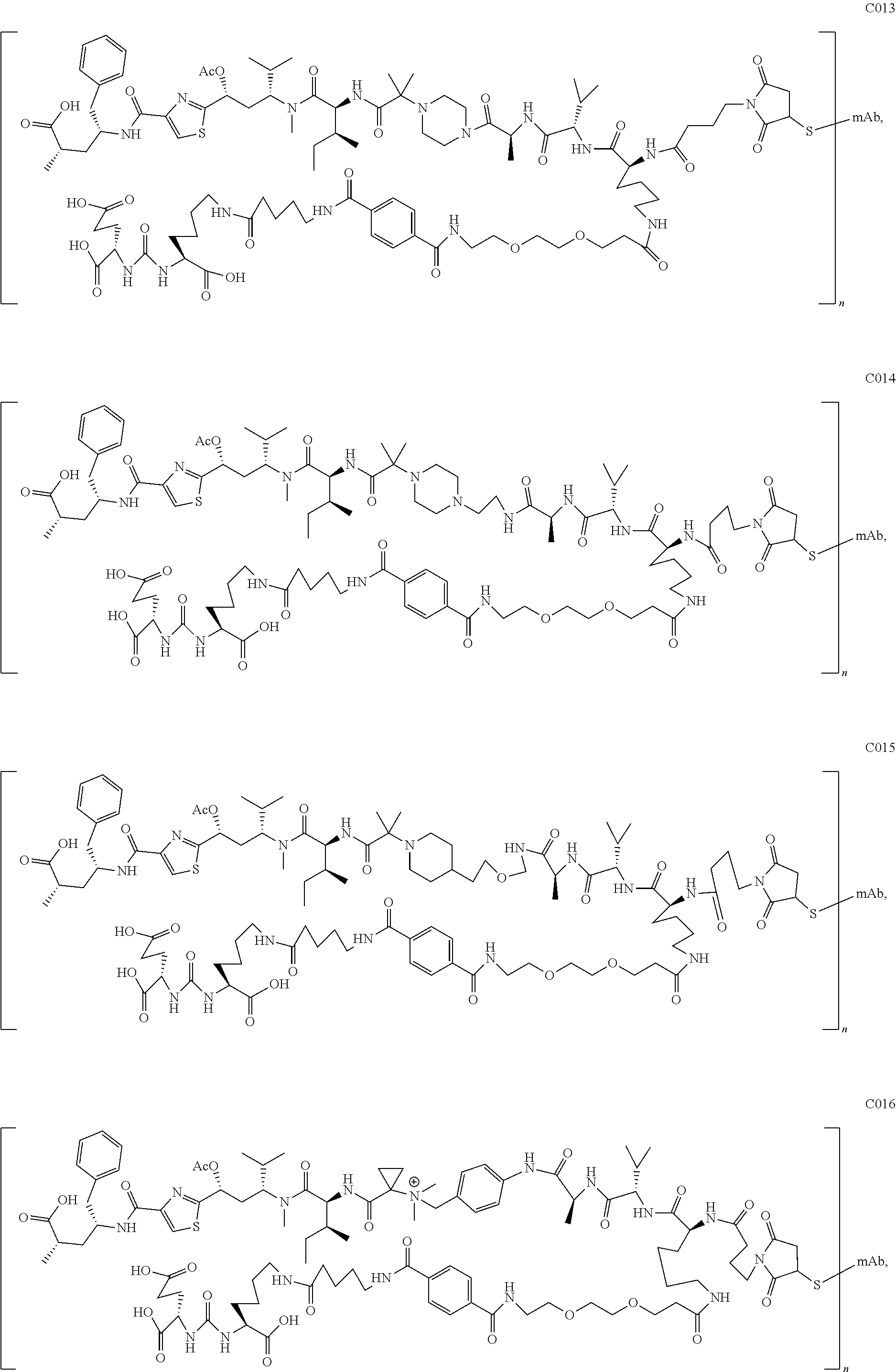

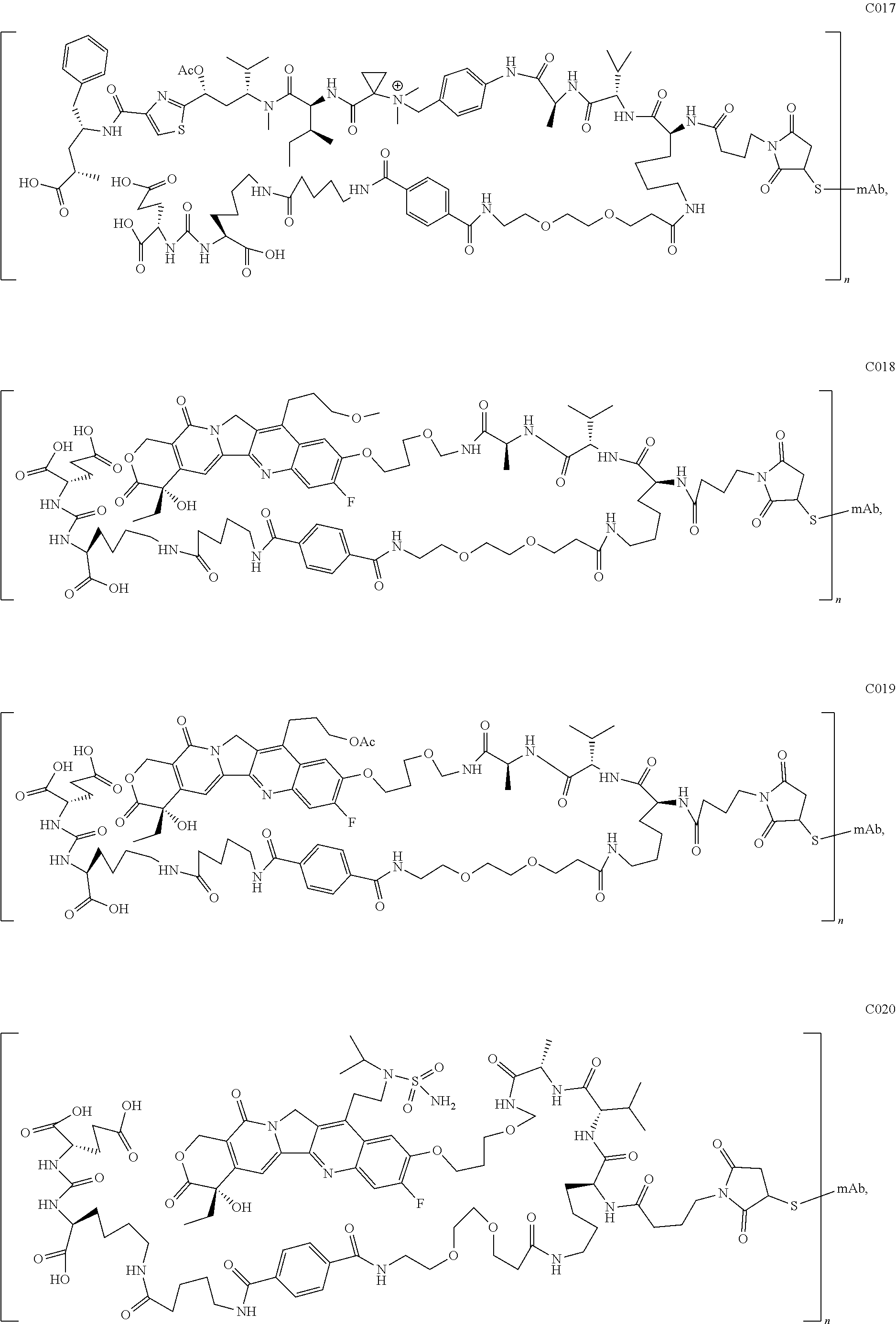

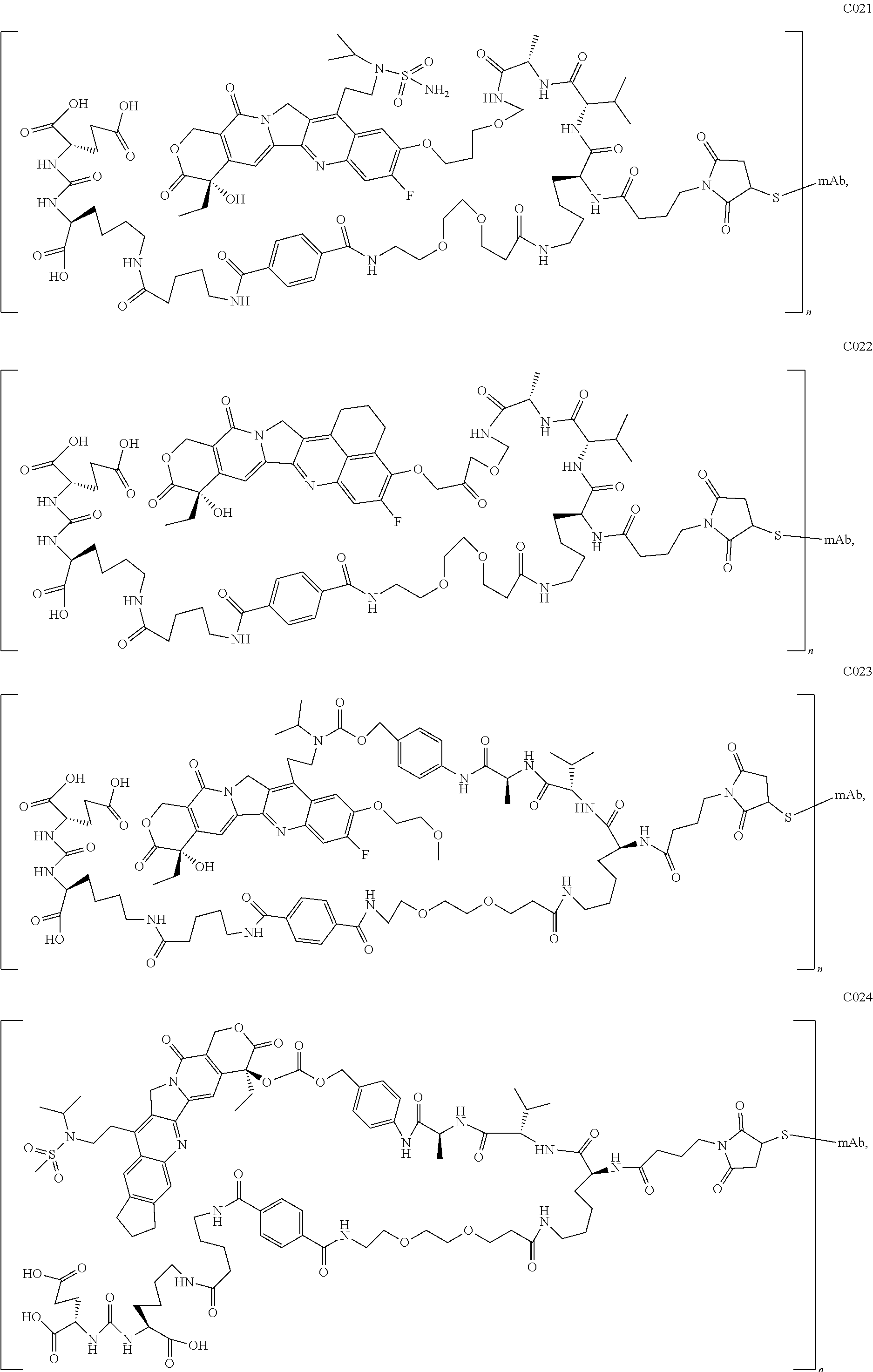

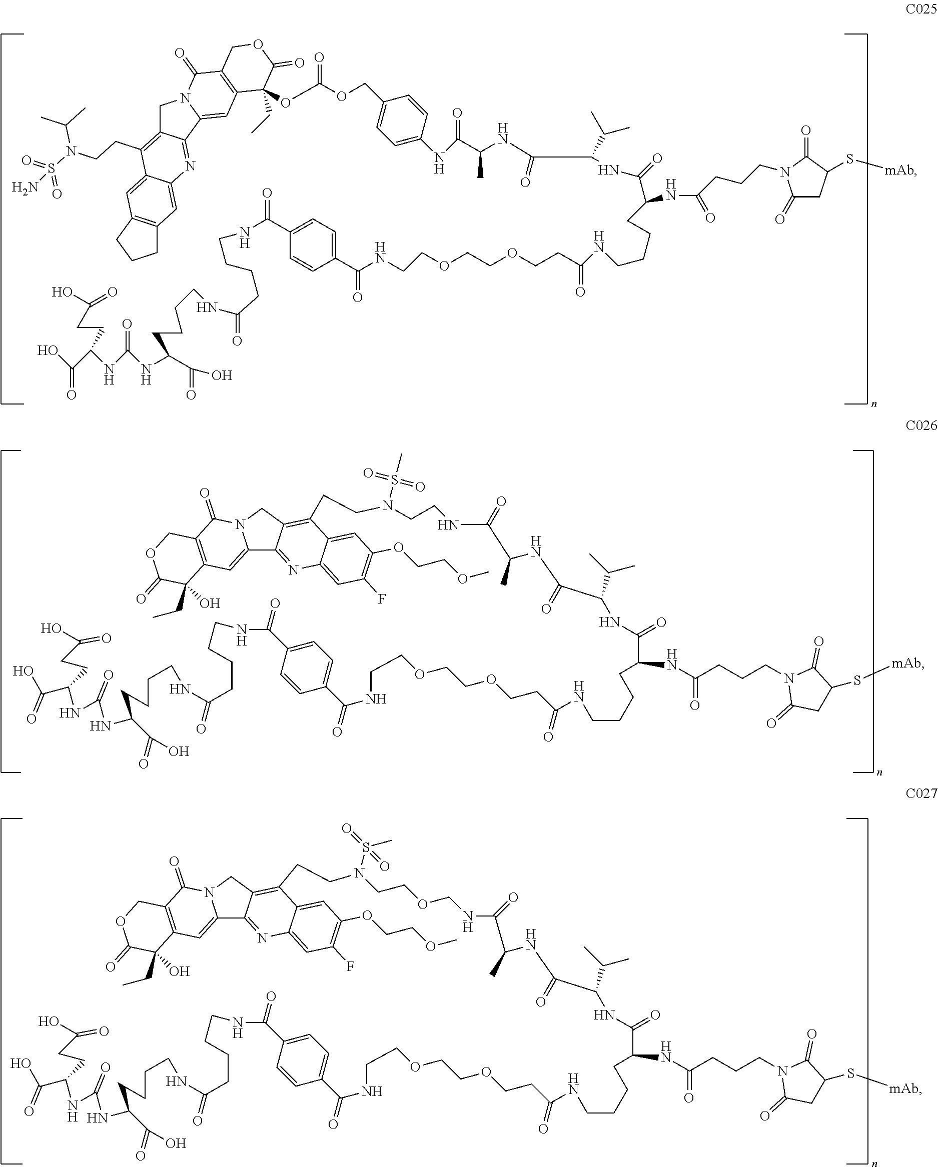

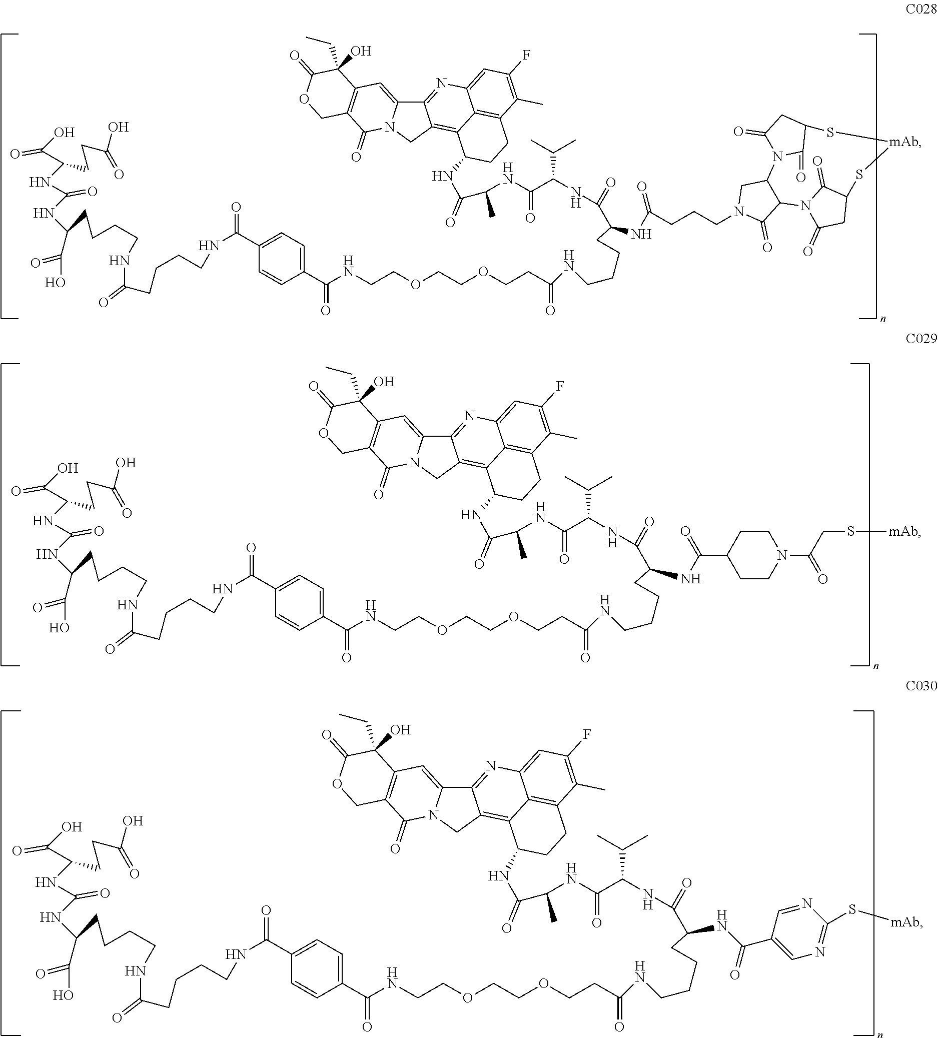

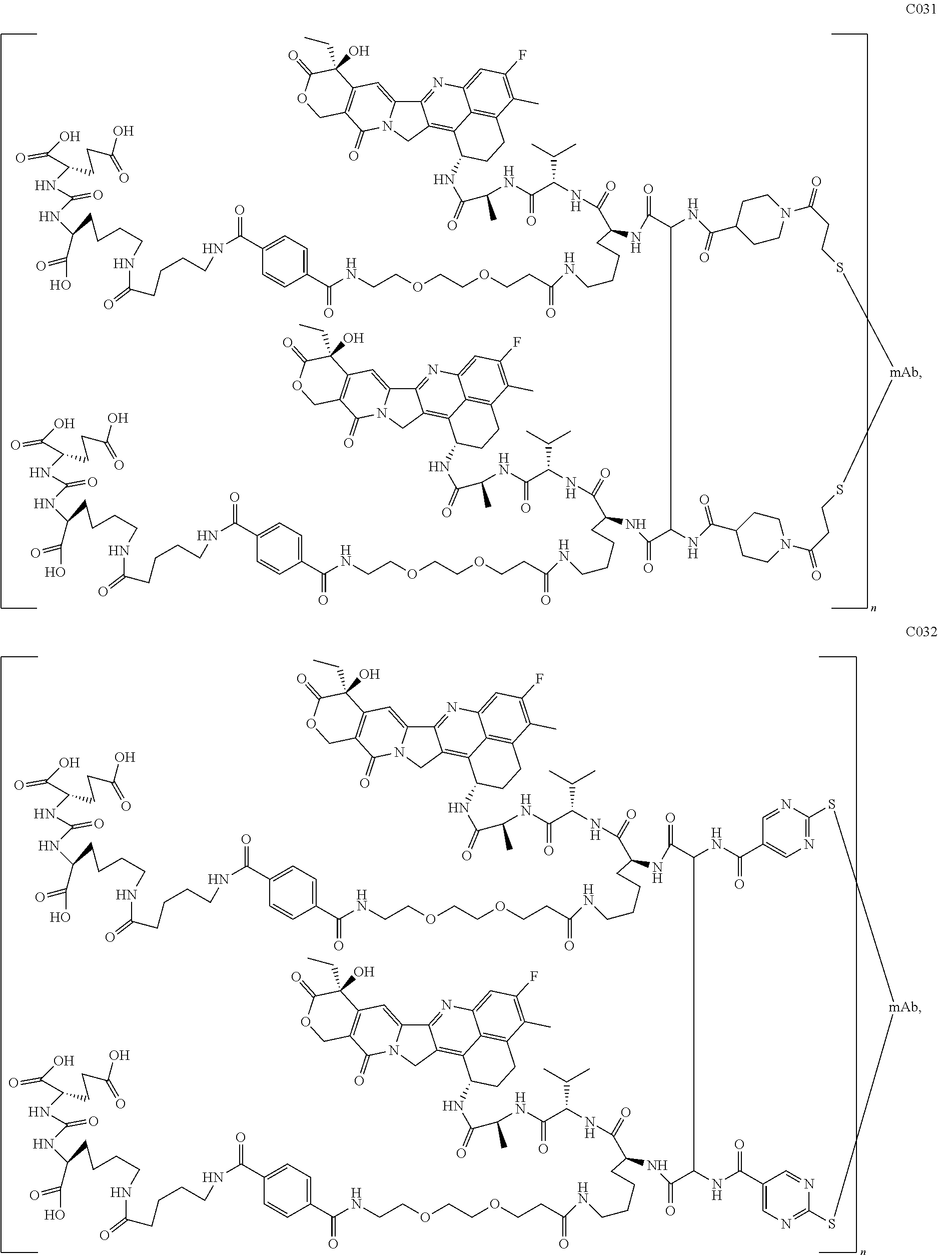

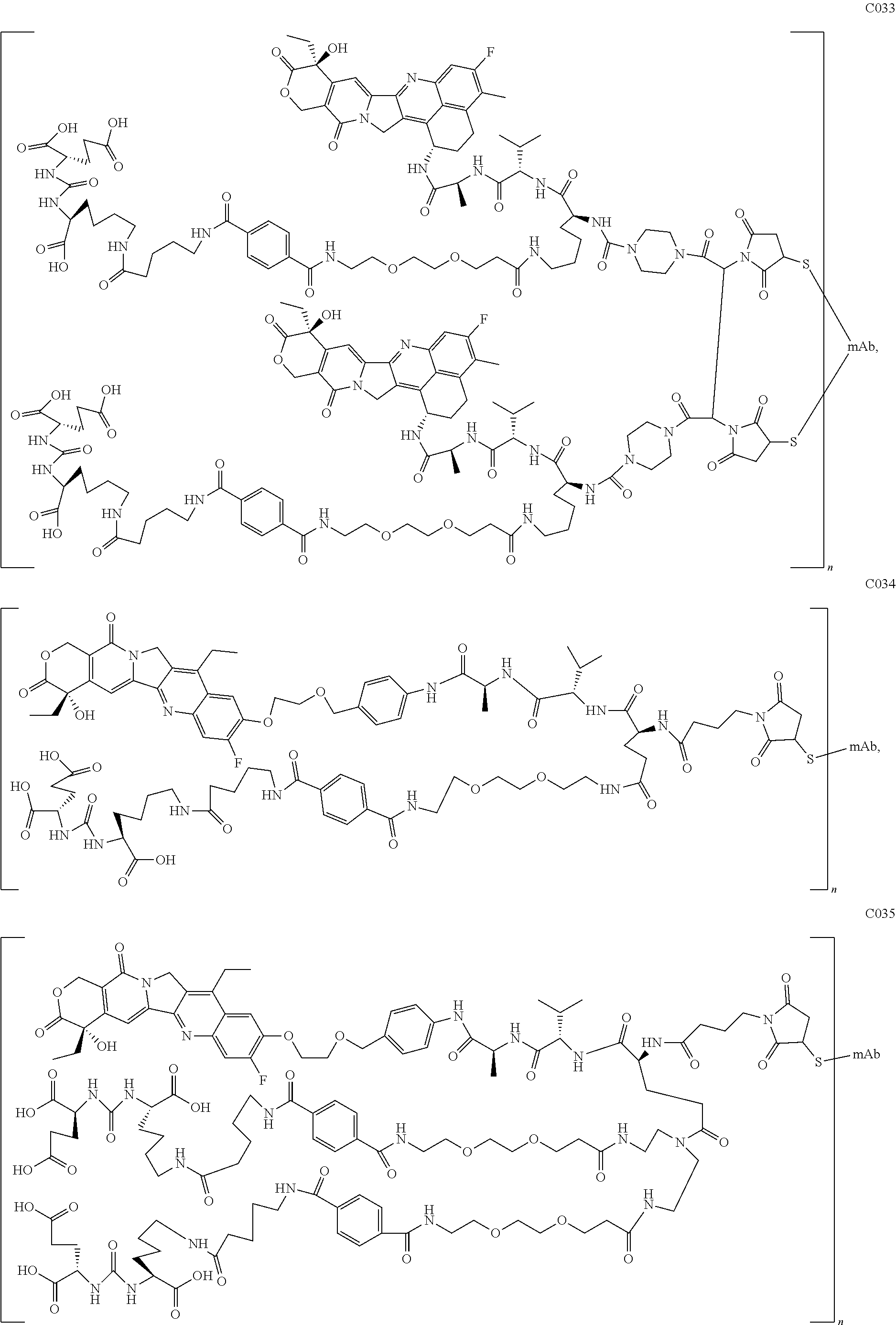

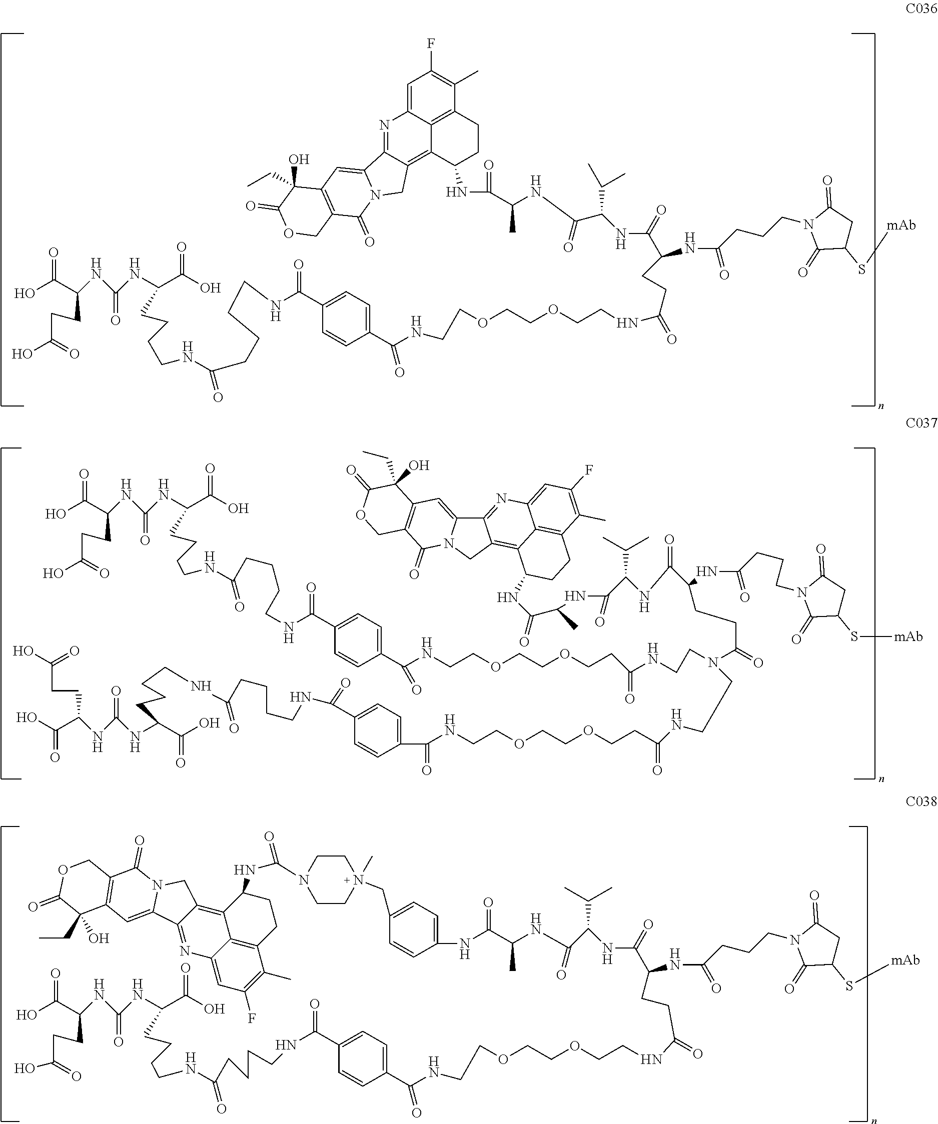

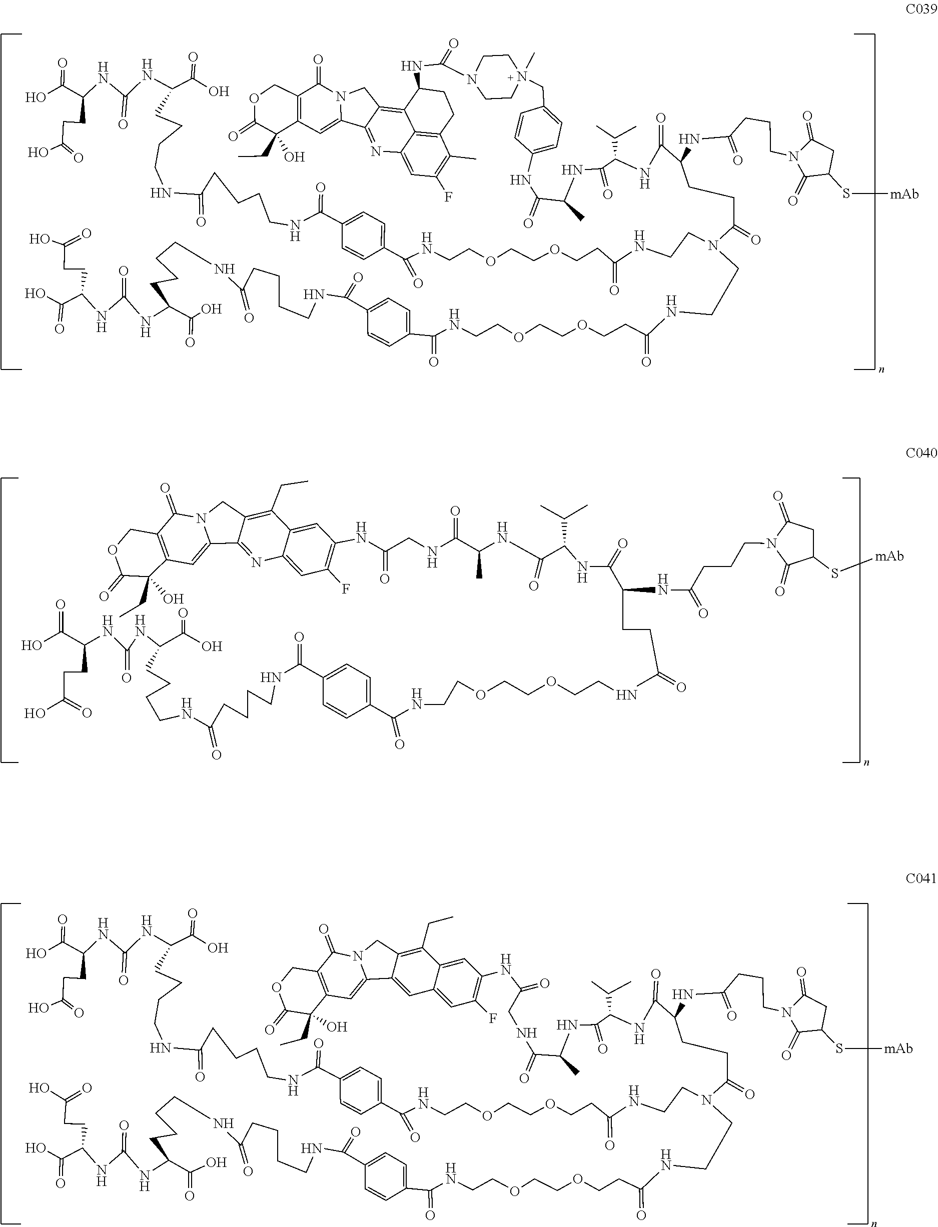

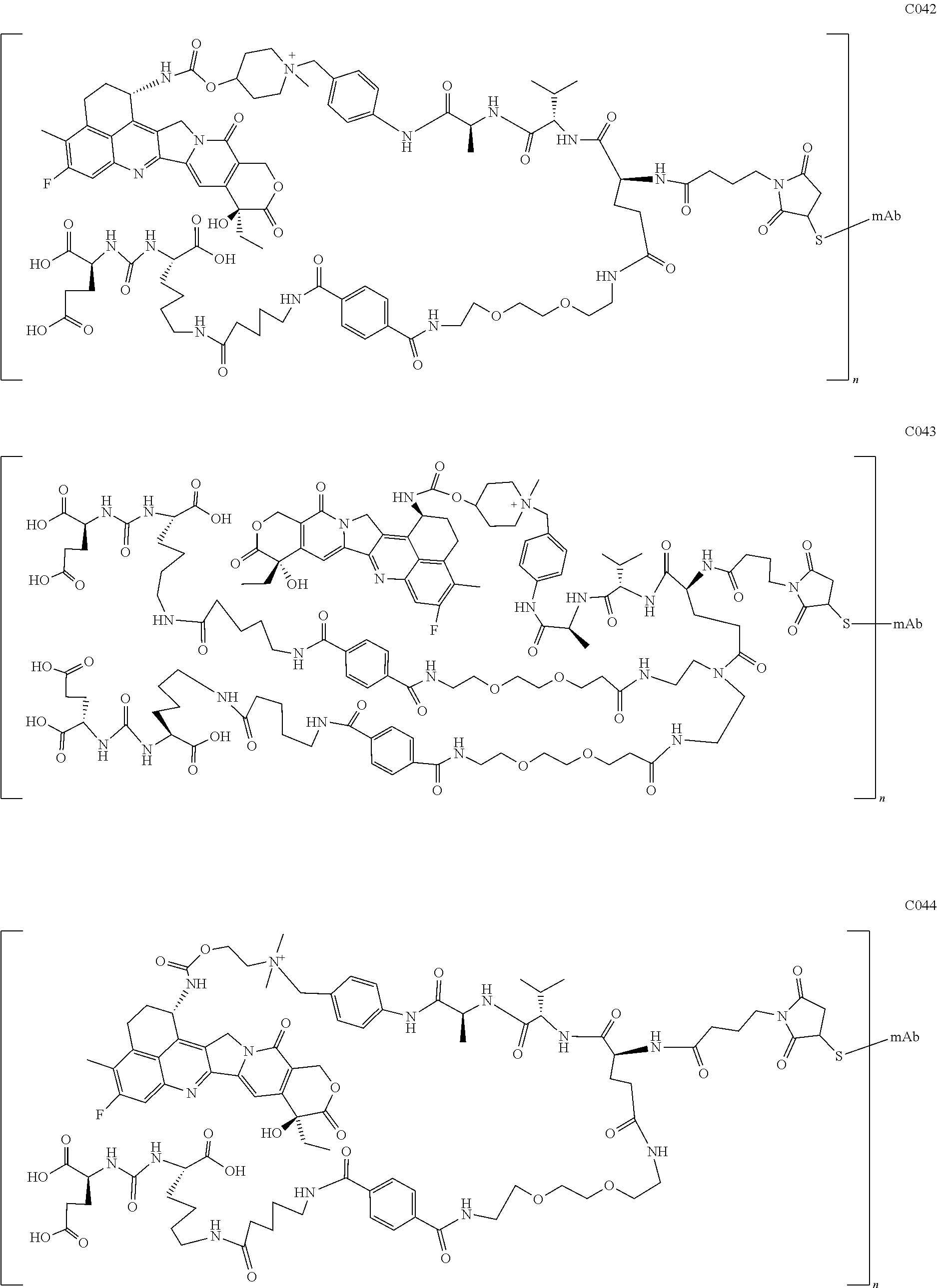

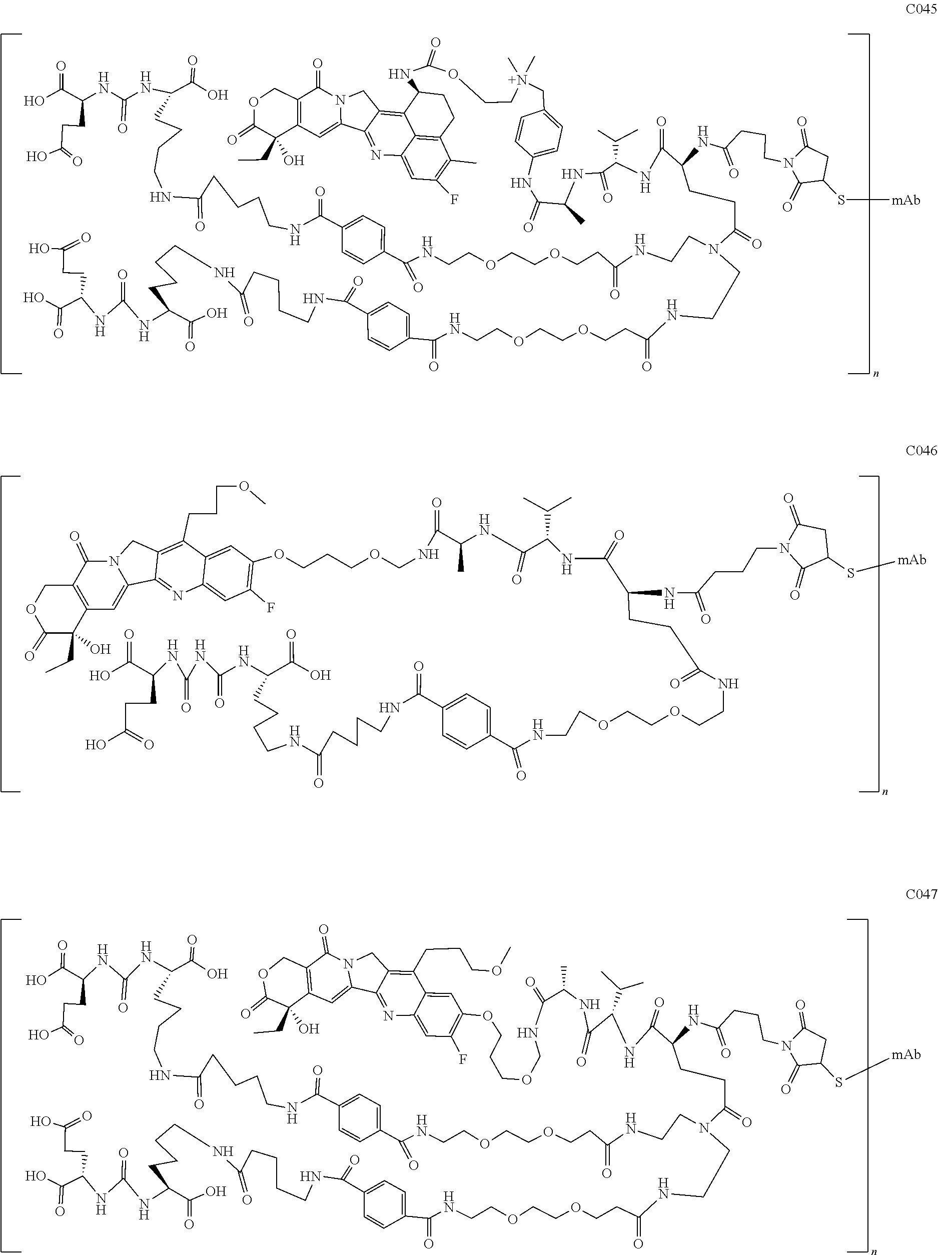

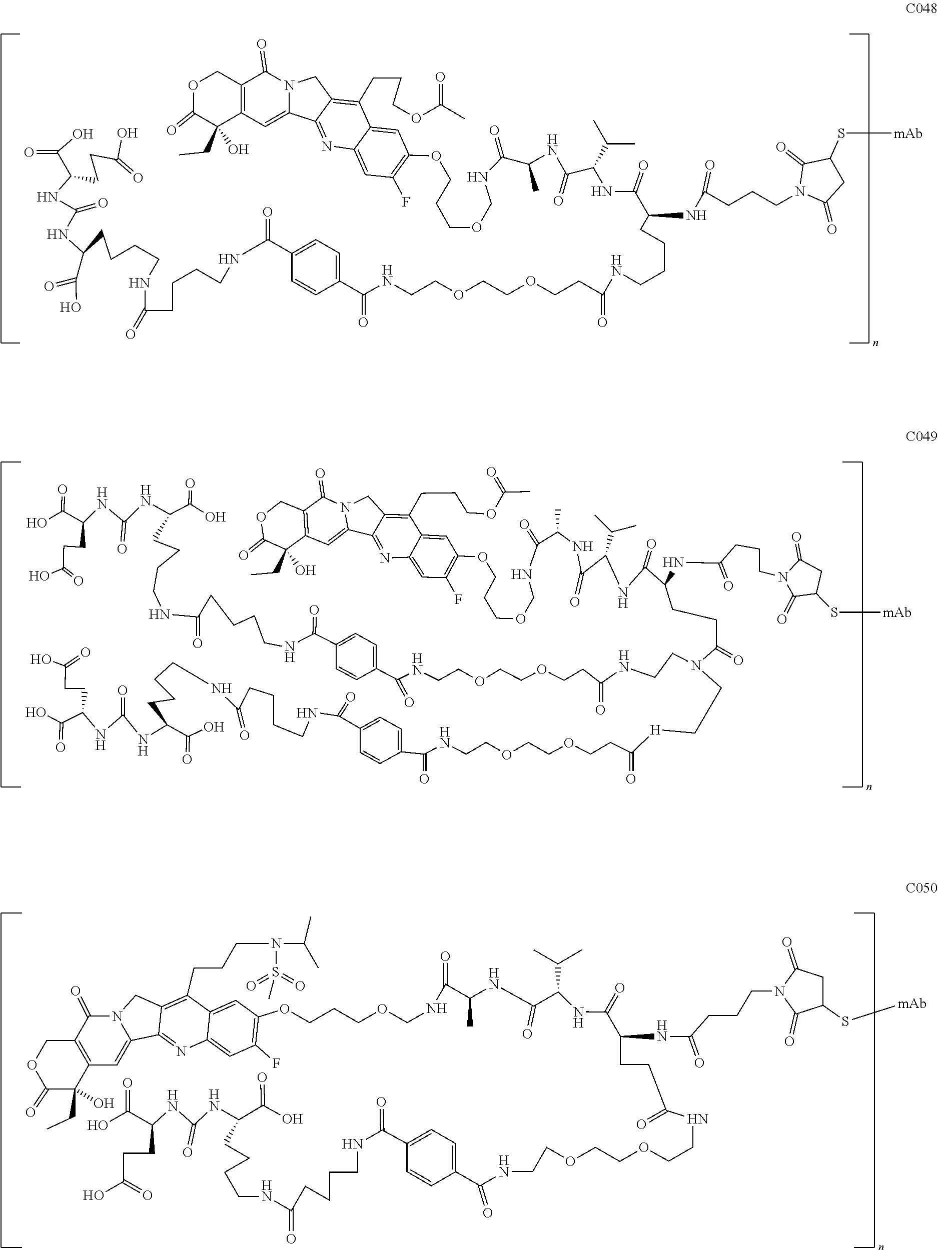

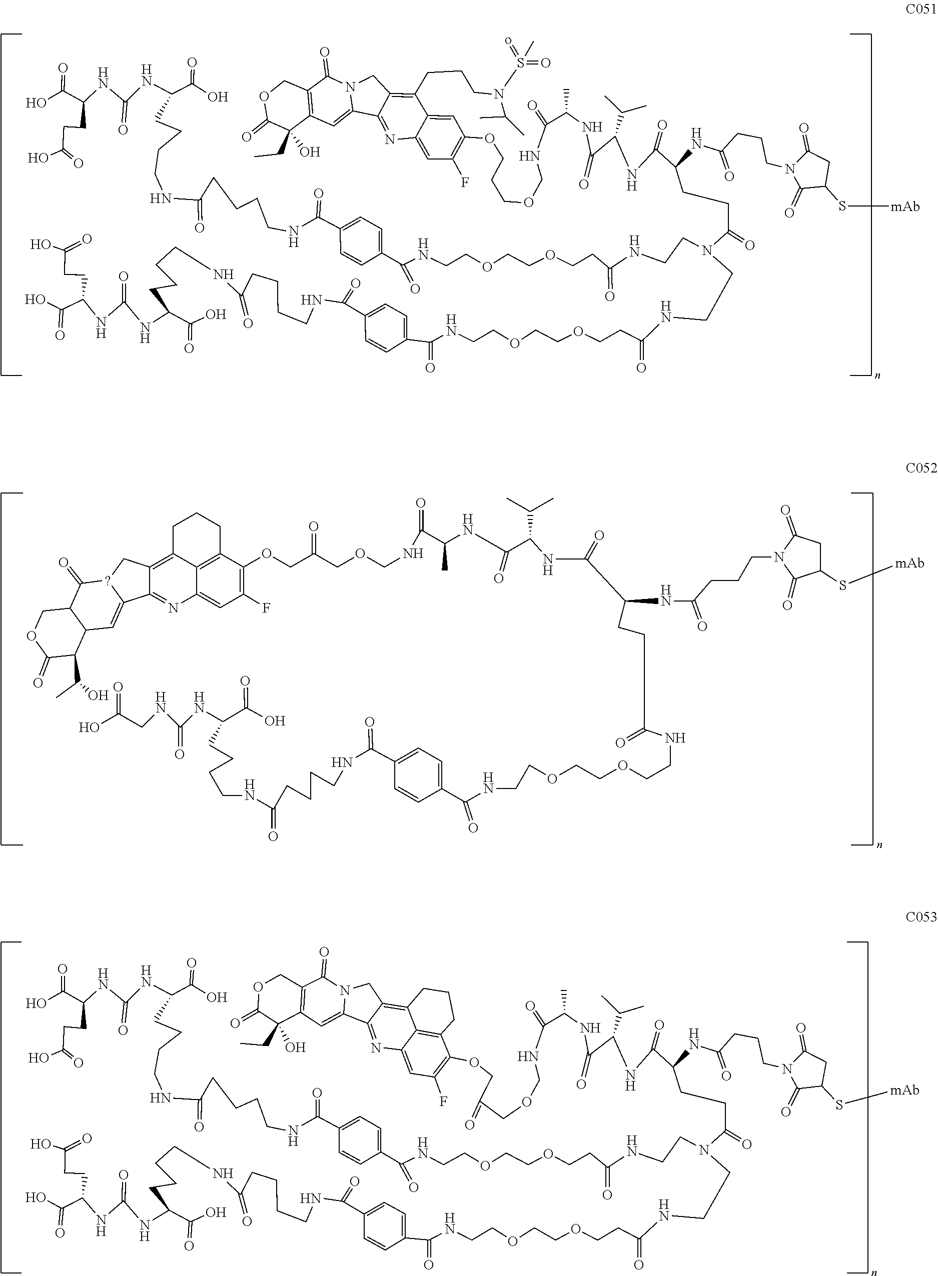

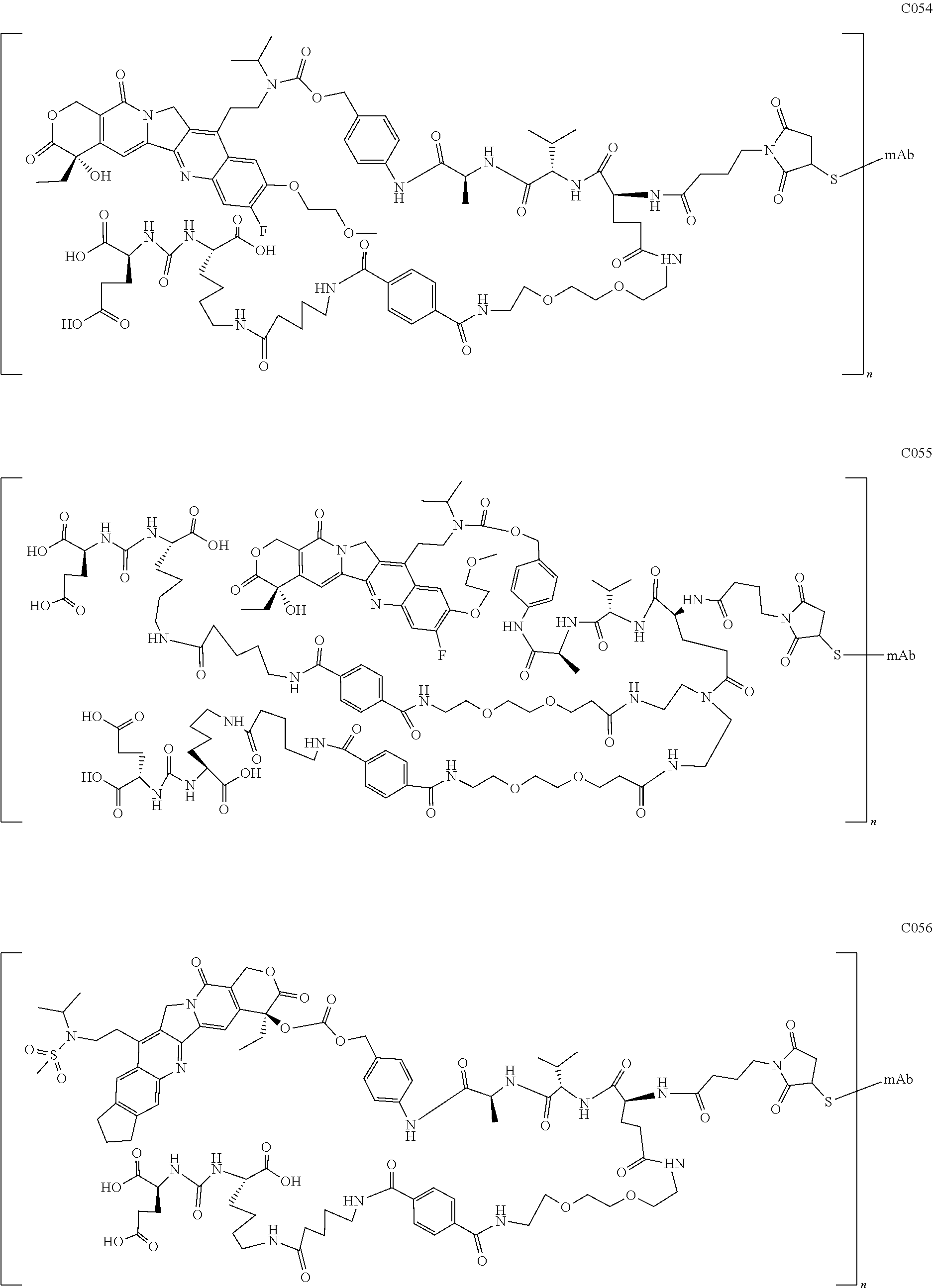

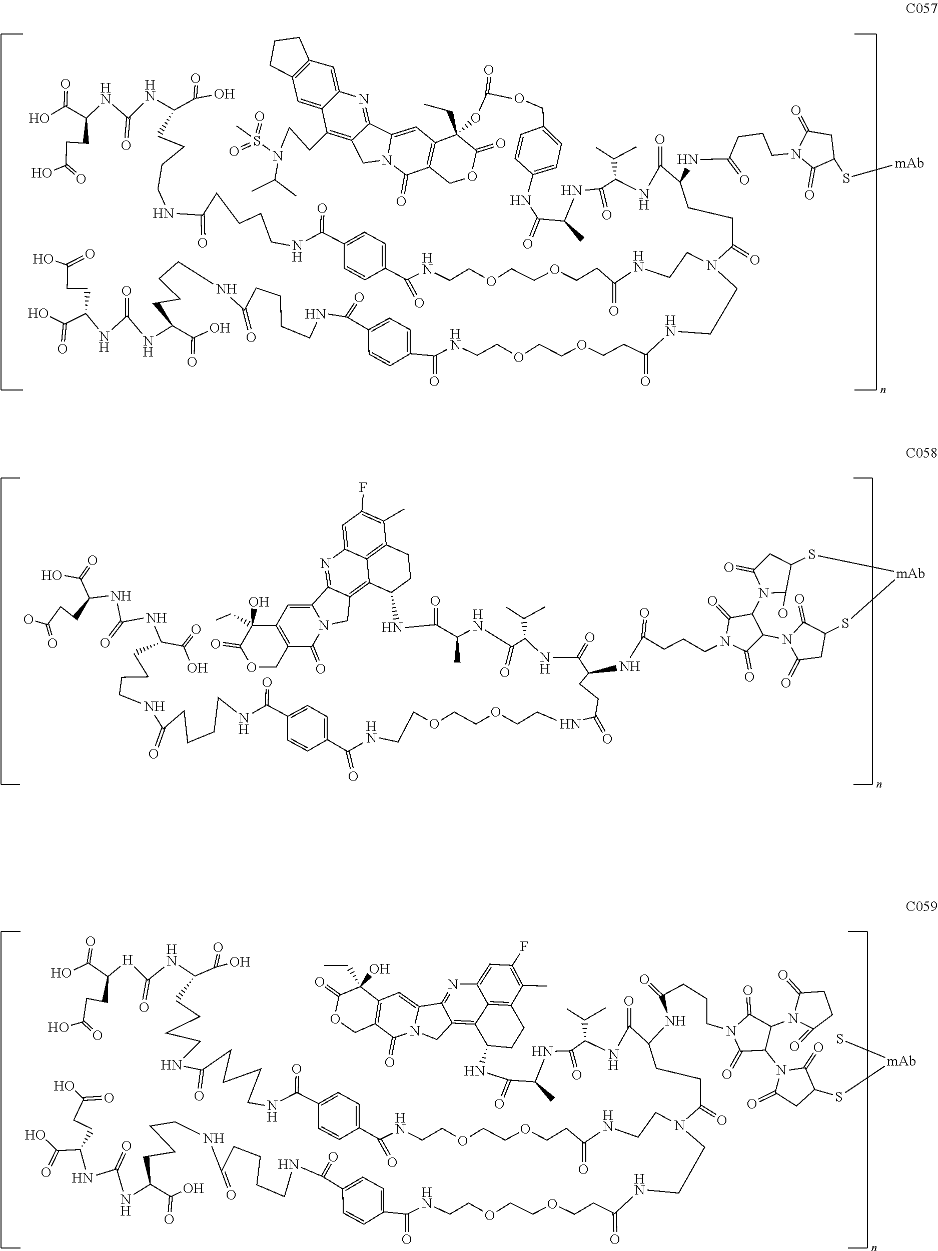

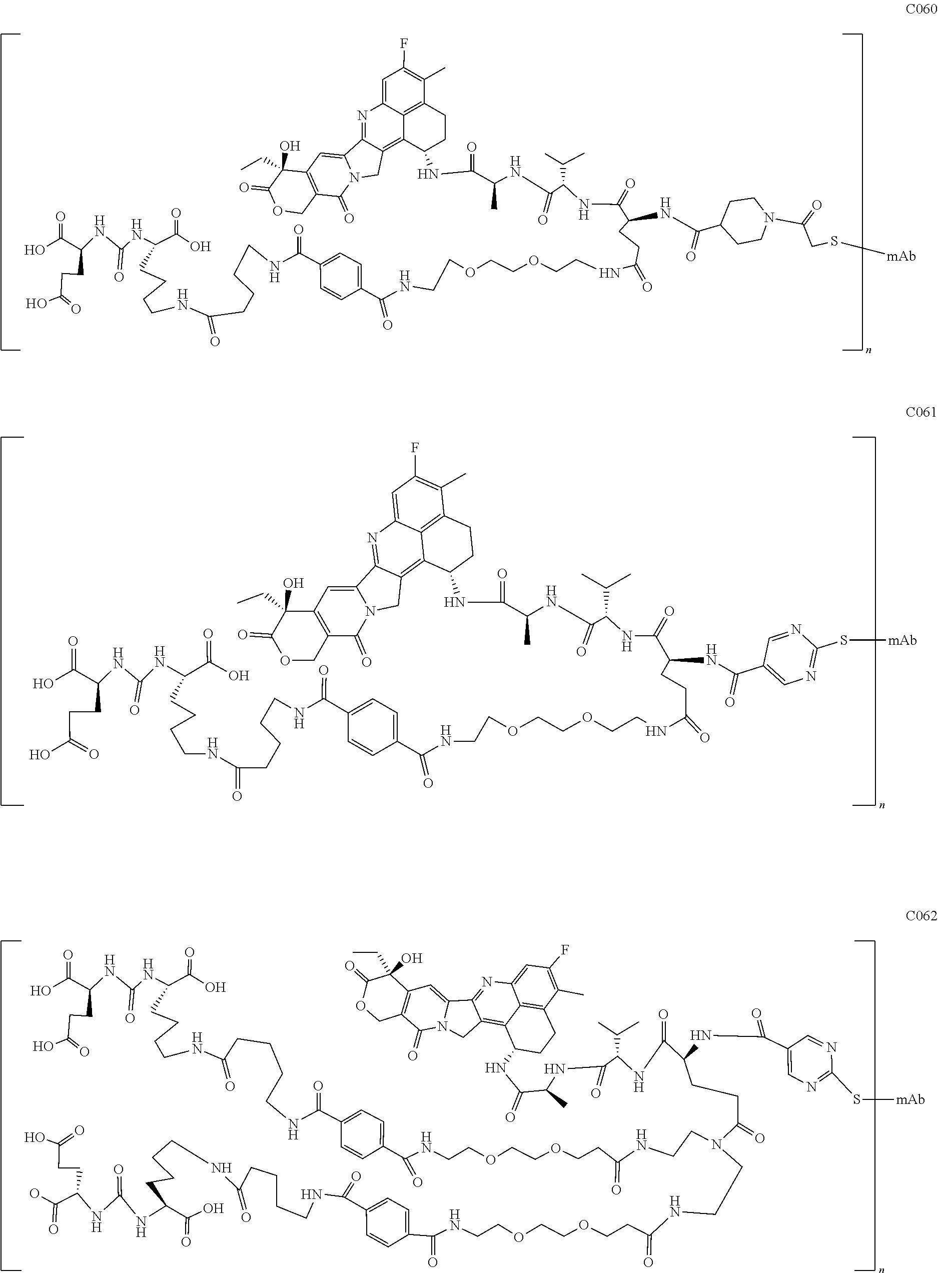

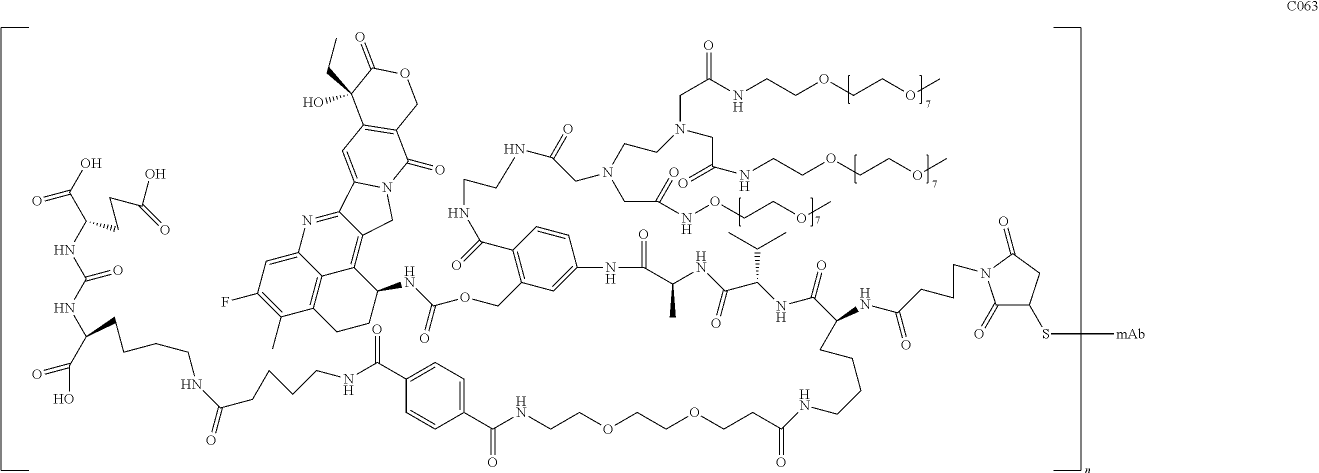

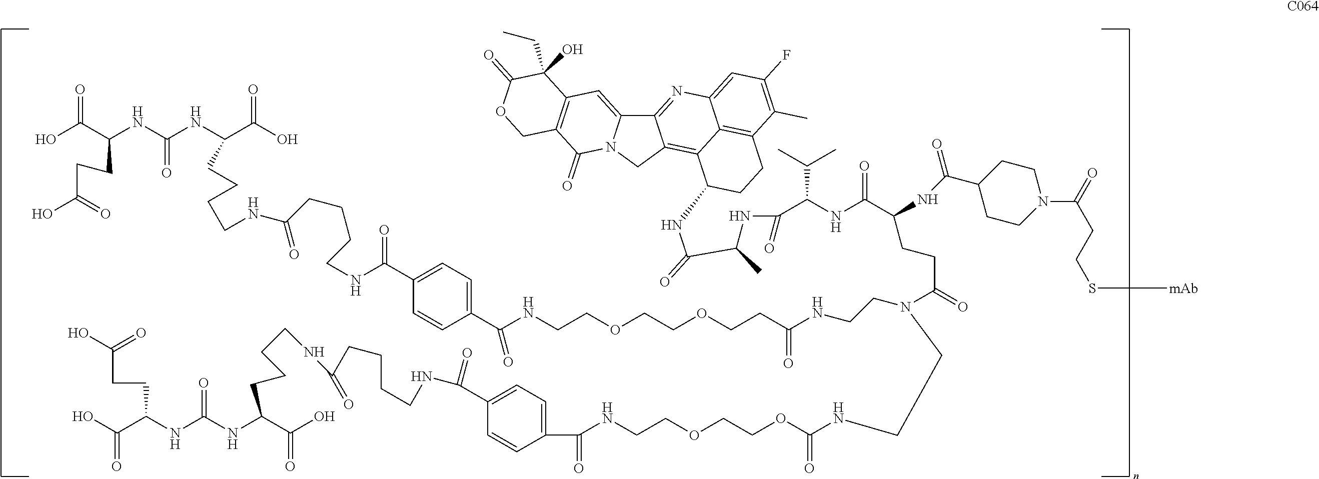

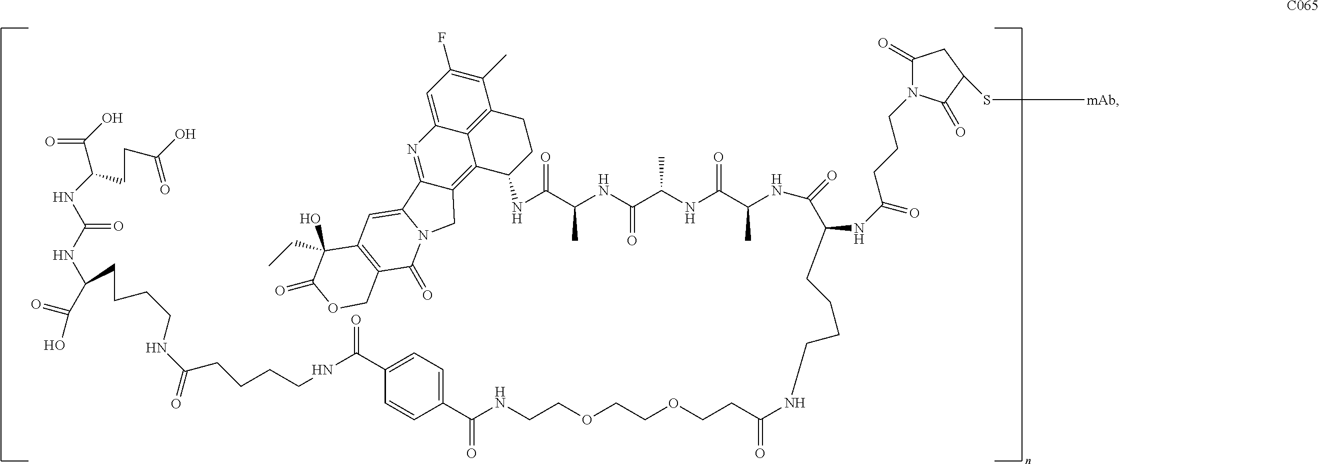

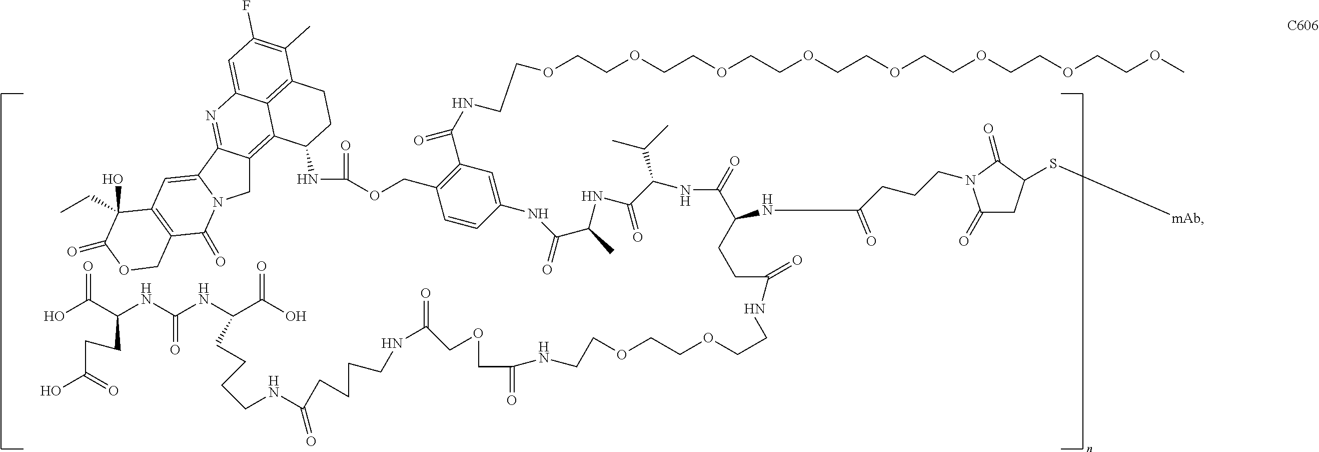

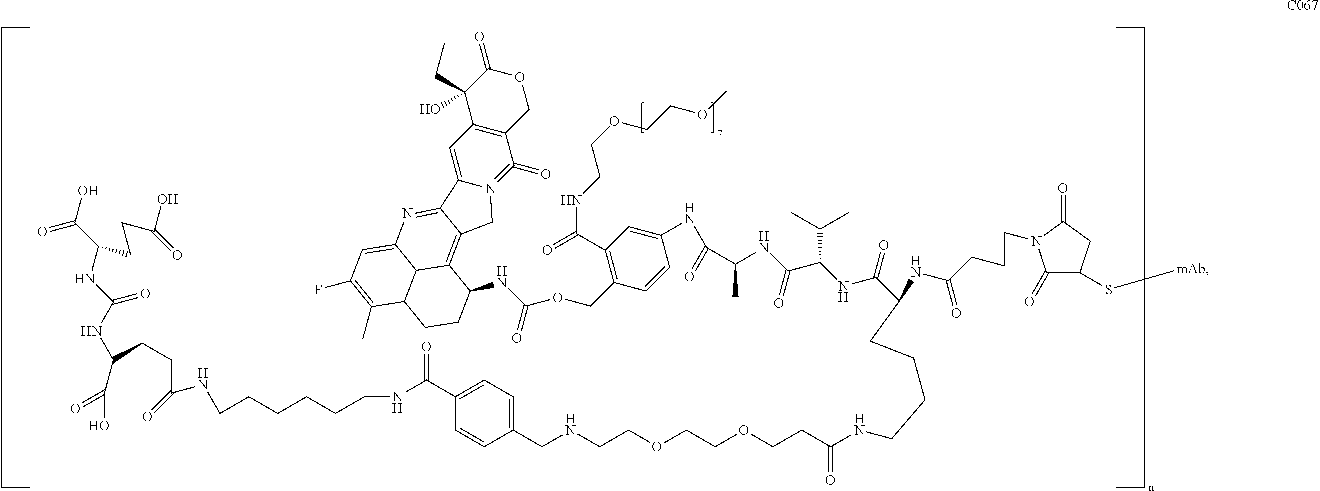

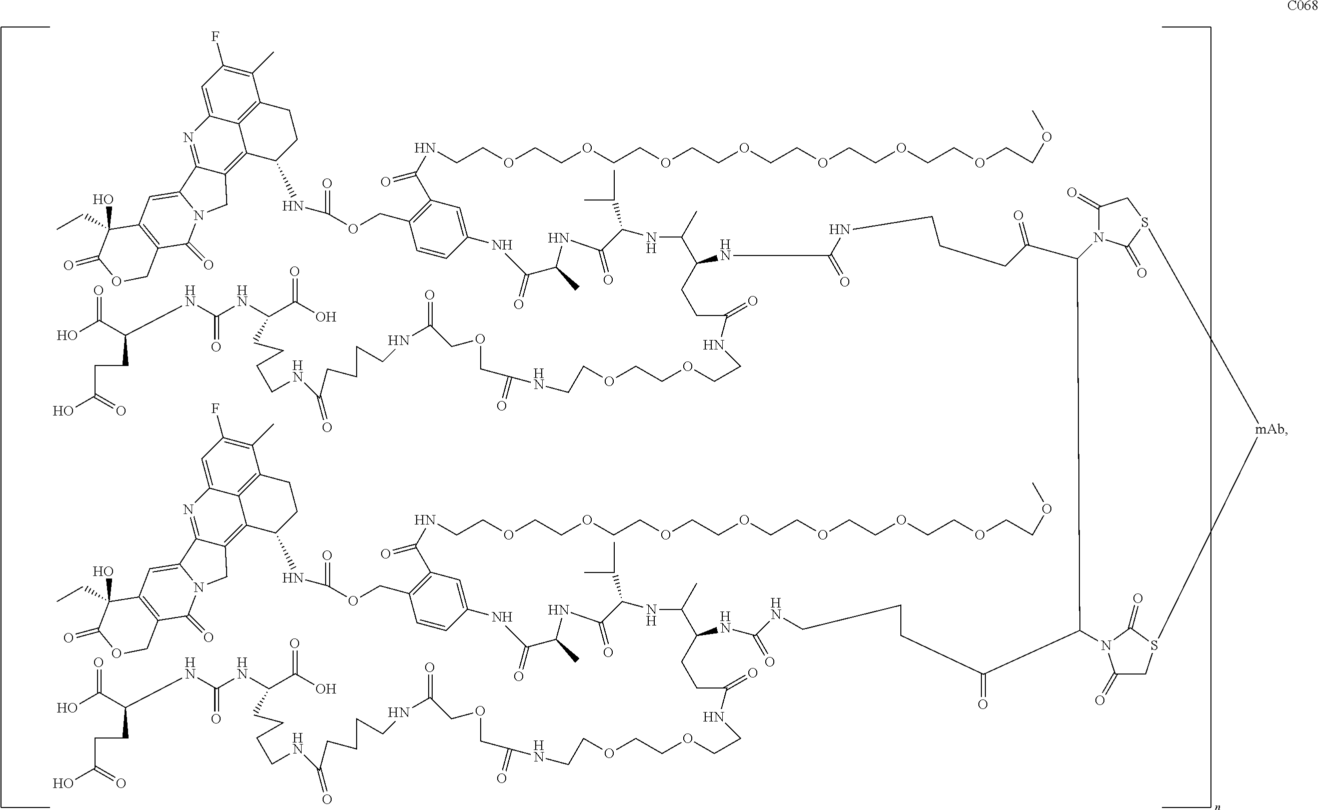

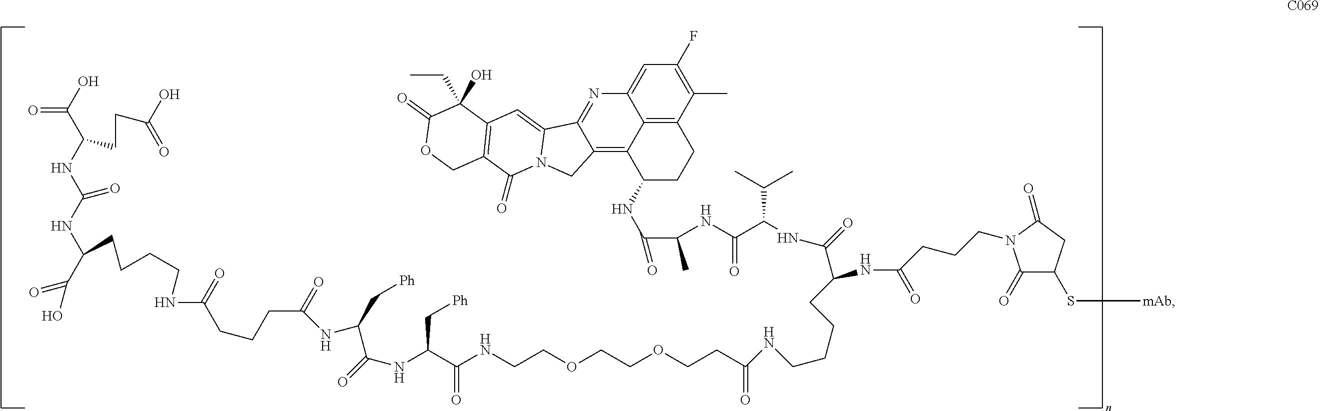

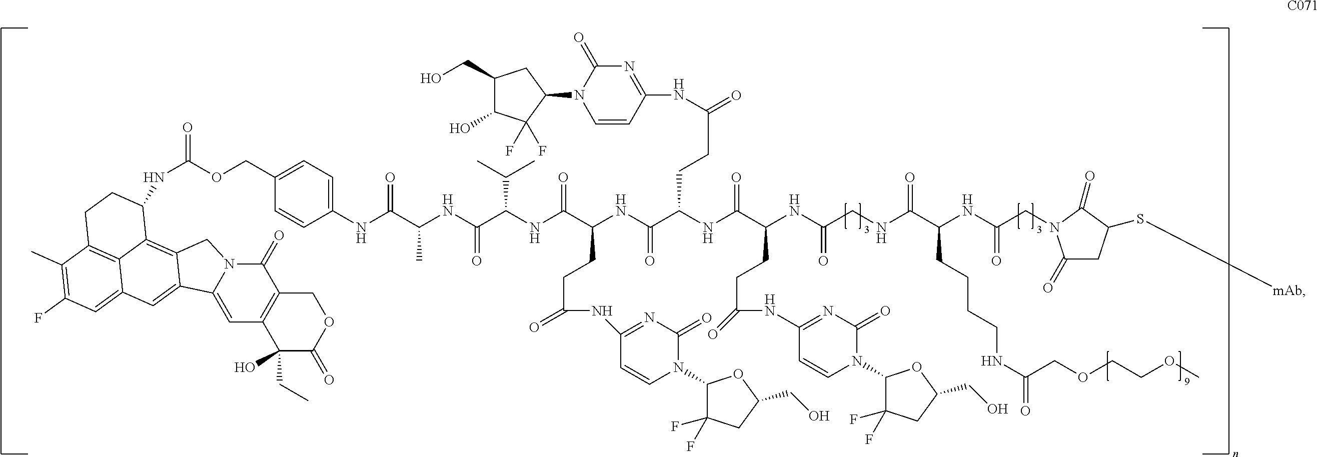

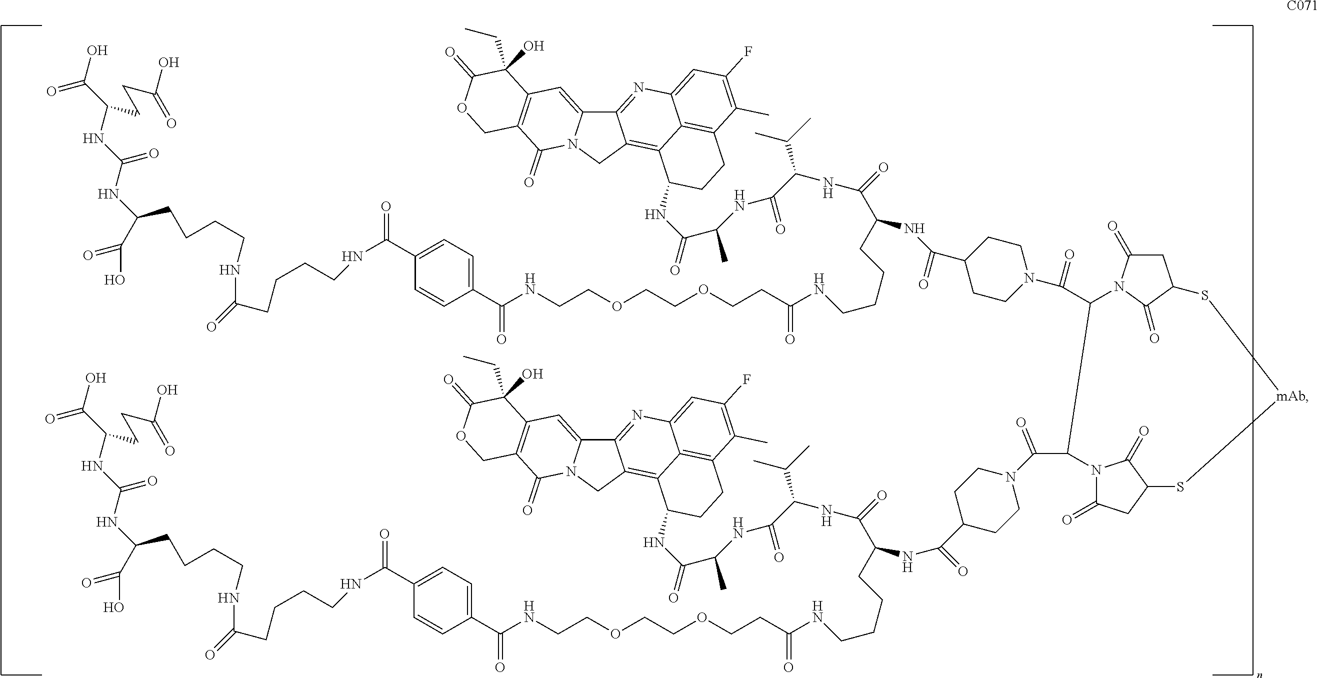

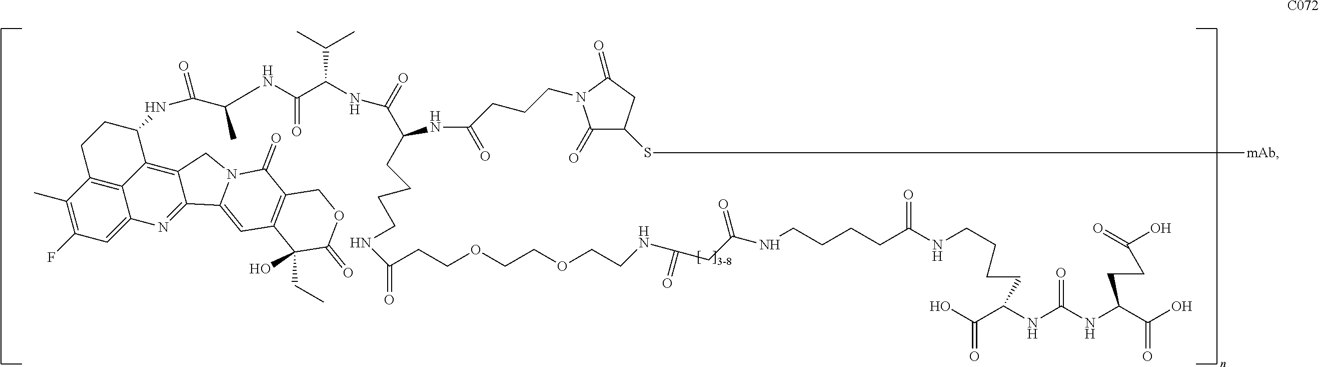

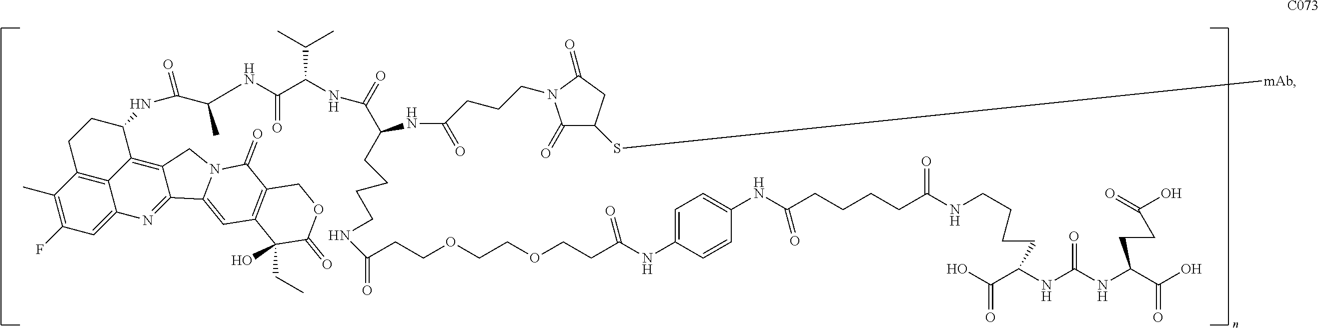

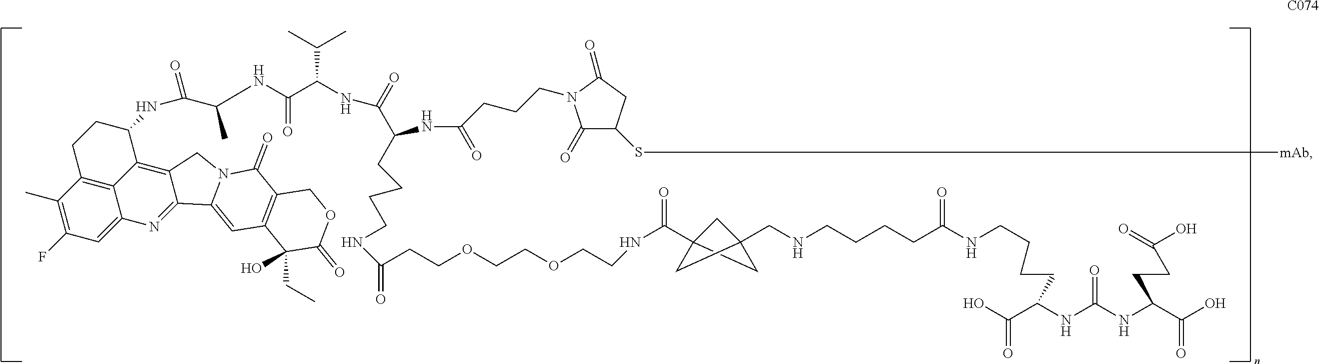

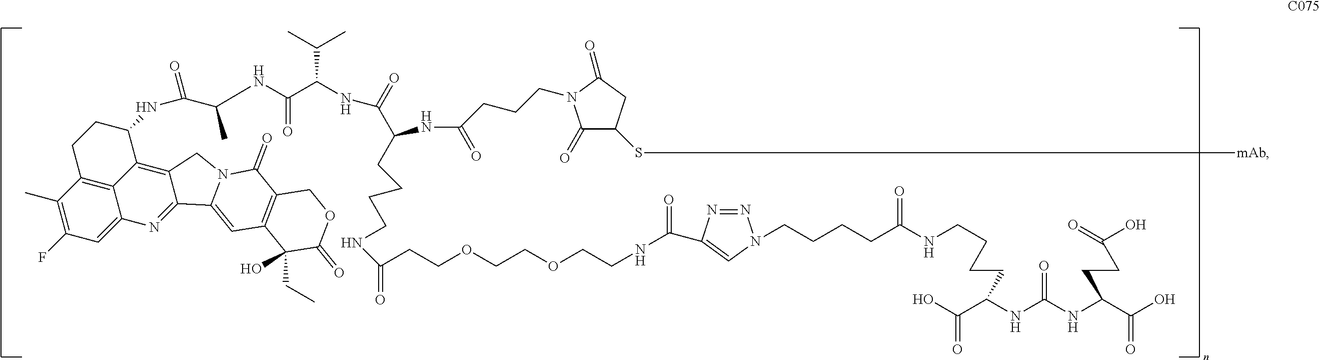

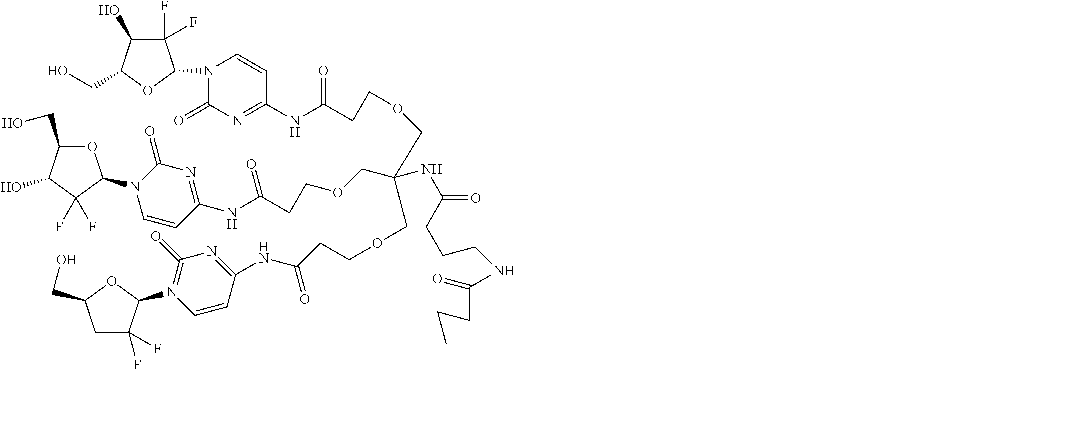

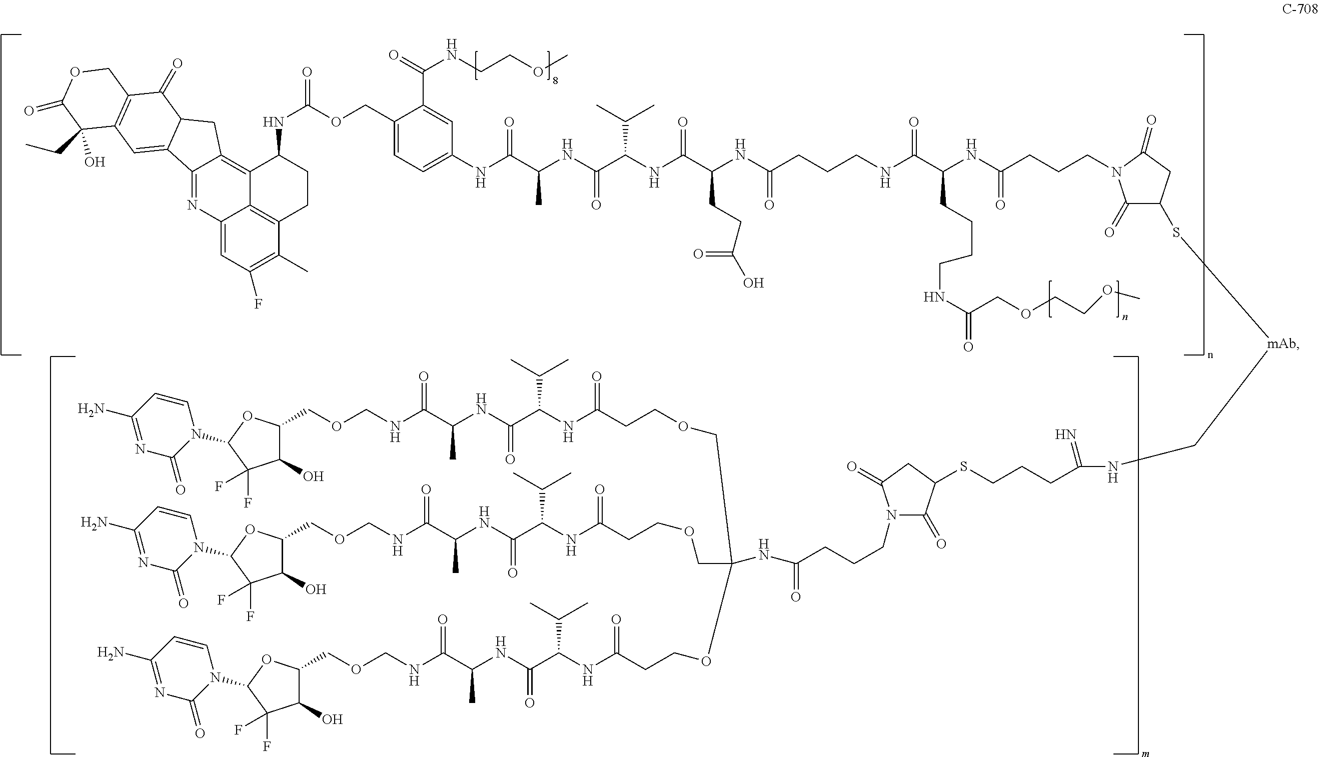

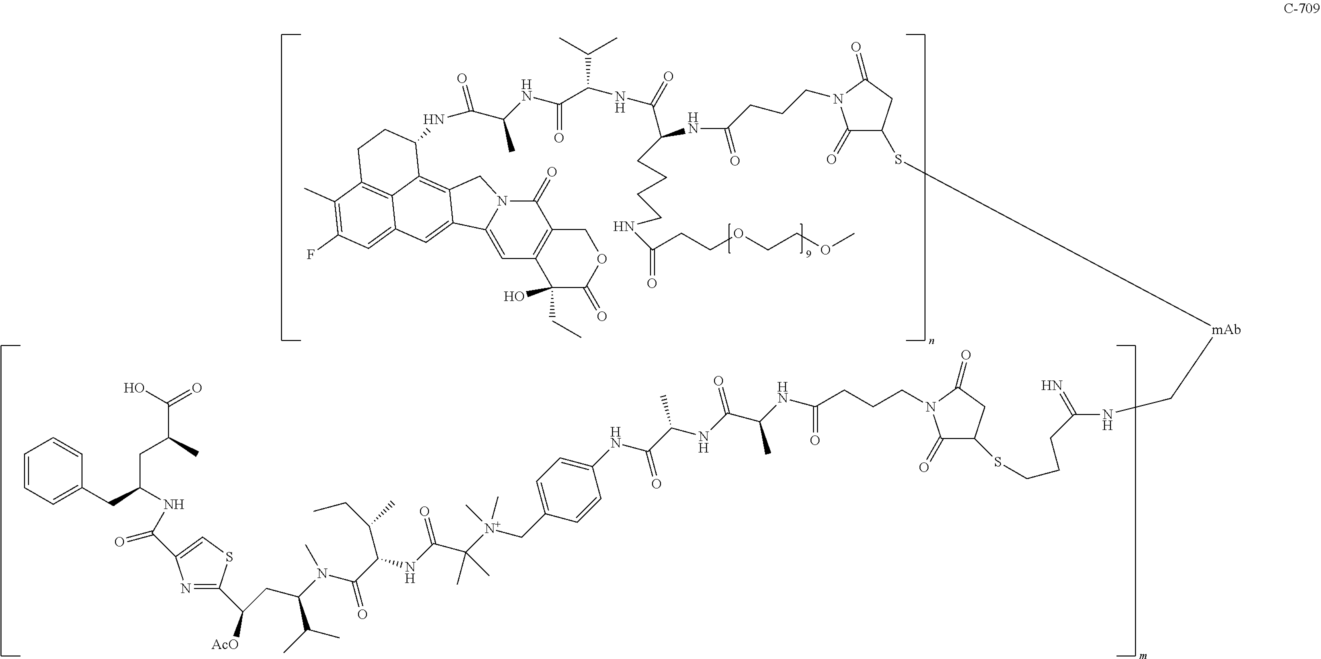

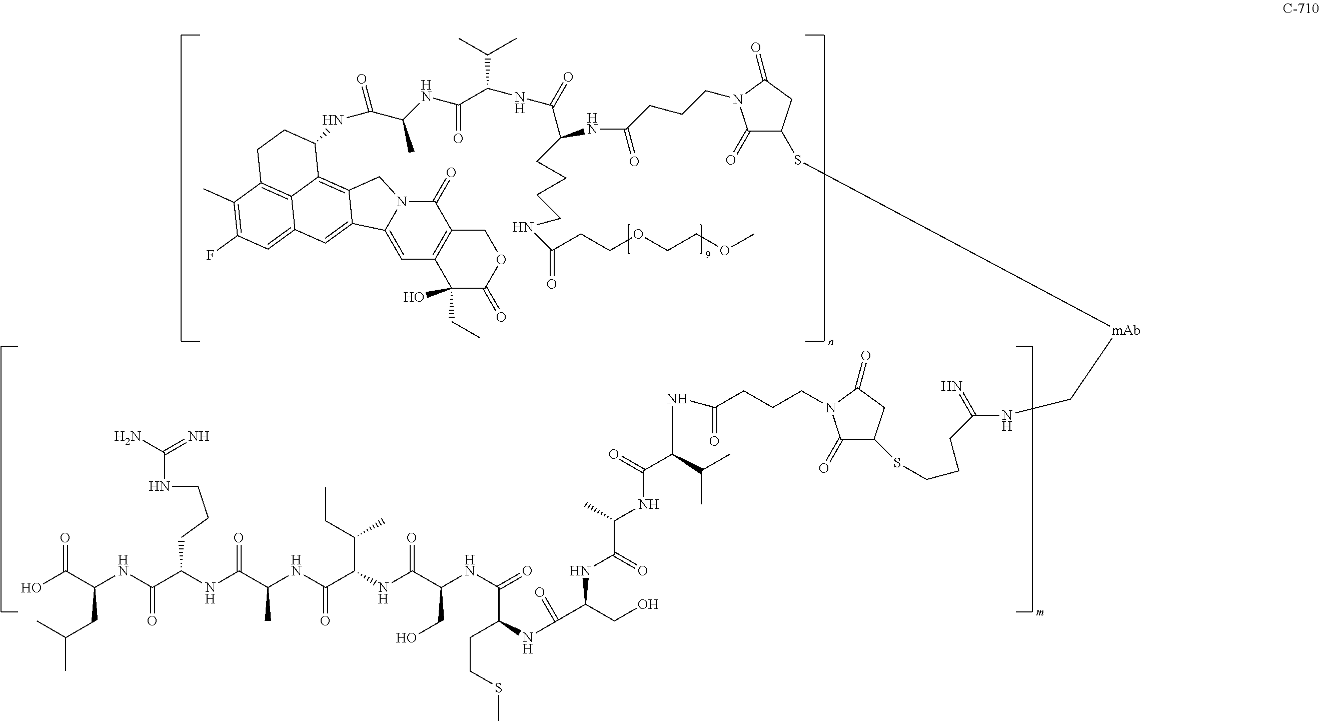

wherein mAb above is an antibody; n and n1 are 1˜30, preferably 1˜20, more preferably 2˜8 (2, 2, 3, 4, 5, 6, 7, 8, and these number can have decimals).

In certain embodiments, the conjugates of Formula (I), (II) and (III) are prepared readily via conjugation reaction of the antibody with compounds having the following formula (V), (VI) and (VII) respectively:

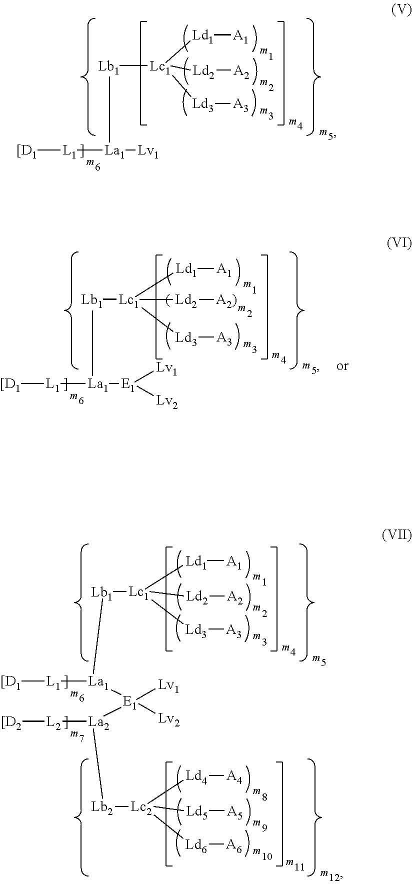

Or the conjugate of Formula (IV) is prepared through sequential conjugation reaction of Formula (V) and Formula (V′) to an antibody:

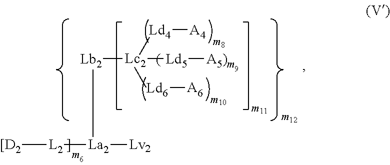

Wherein D₁, D₂, L₁, L₂, La₁, La₂, Lb₁, Lb₂, Lc₁, Lc₂, Ld₁, Ld₂, Ld₃, Ld₄, Ld₅, Ld₆, A₁, A₂, A₃, A₄, A₅, A₆, E₁, m₁, m₂, m₃, m₄, m₅, m₆, m₇, m₈, m₉, m₁₀, m₁₁, and m₁₂ are defined the same in the Formula (I), (II) and (III);

Lv₁ and Lv₂ are a reactive group and are independently or jointly selected from:

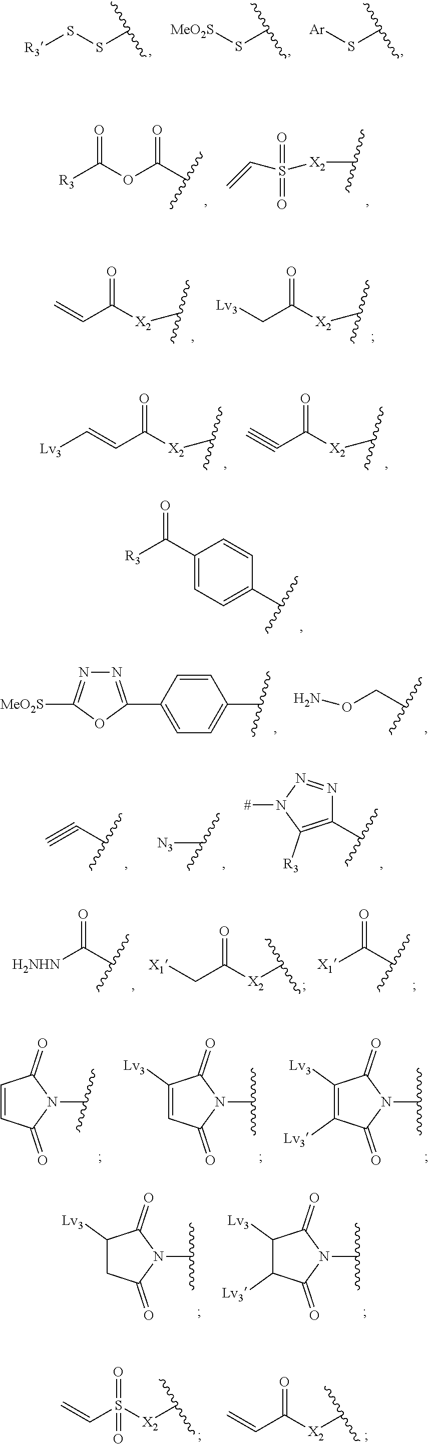

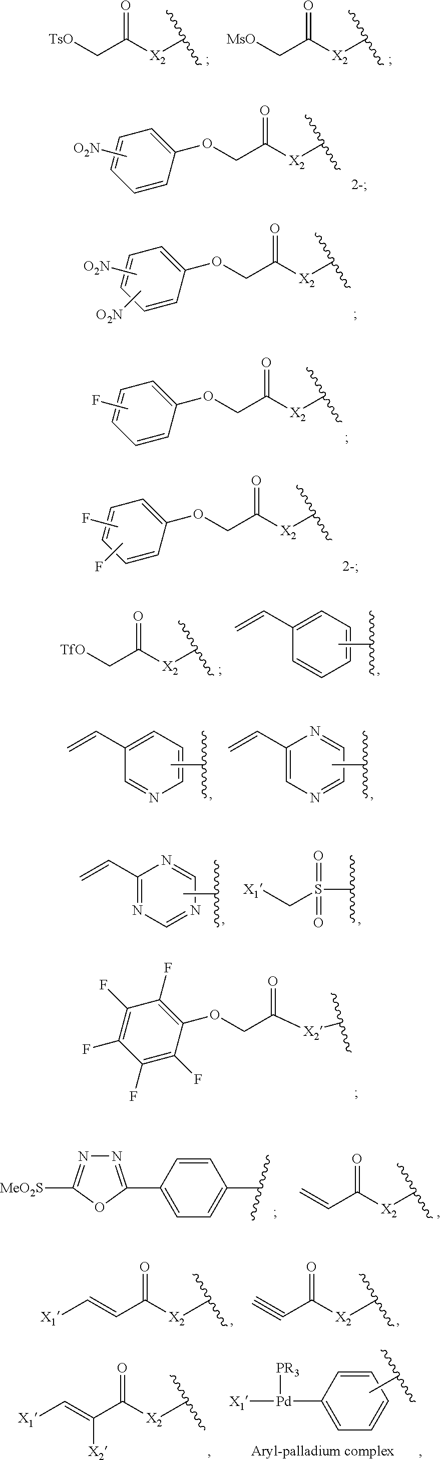

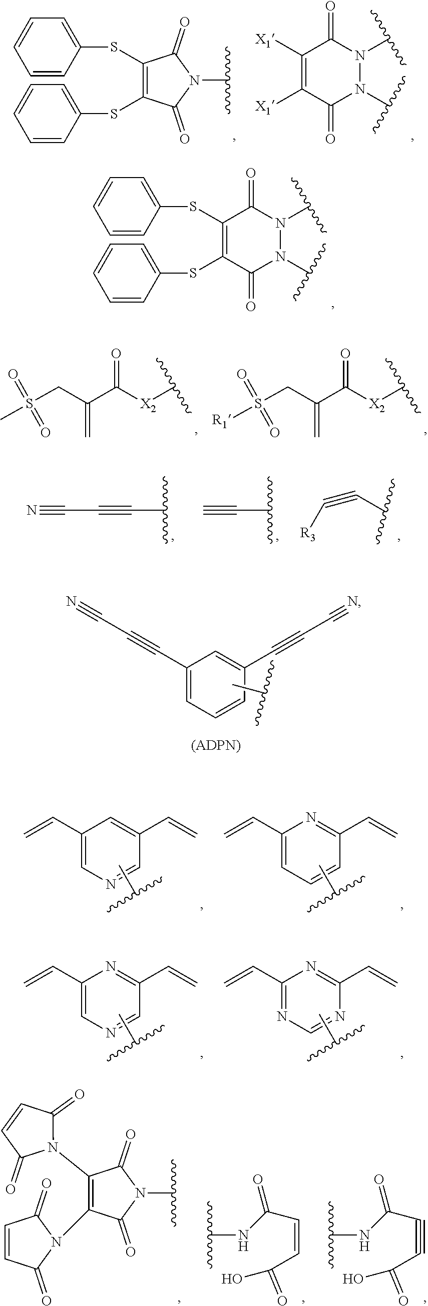

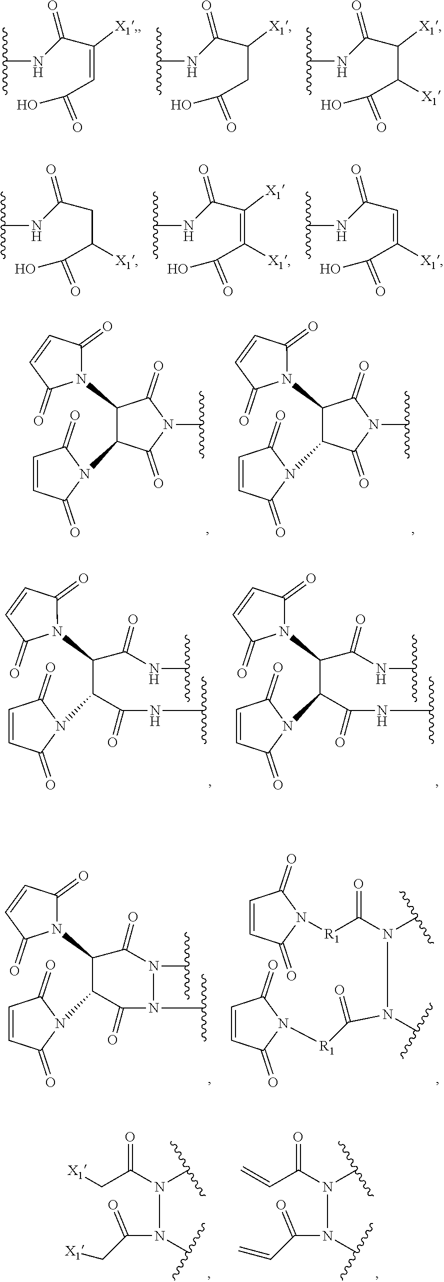

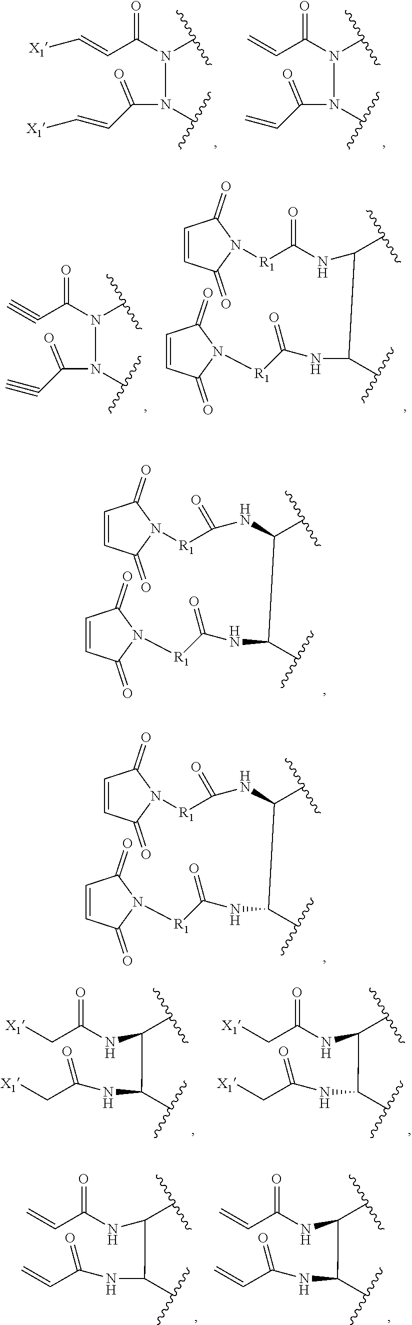

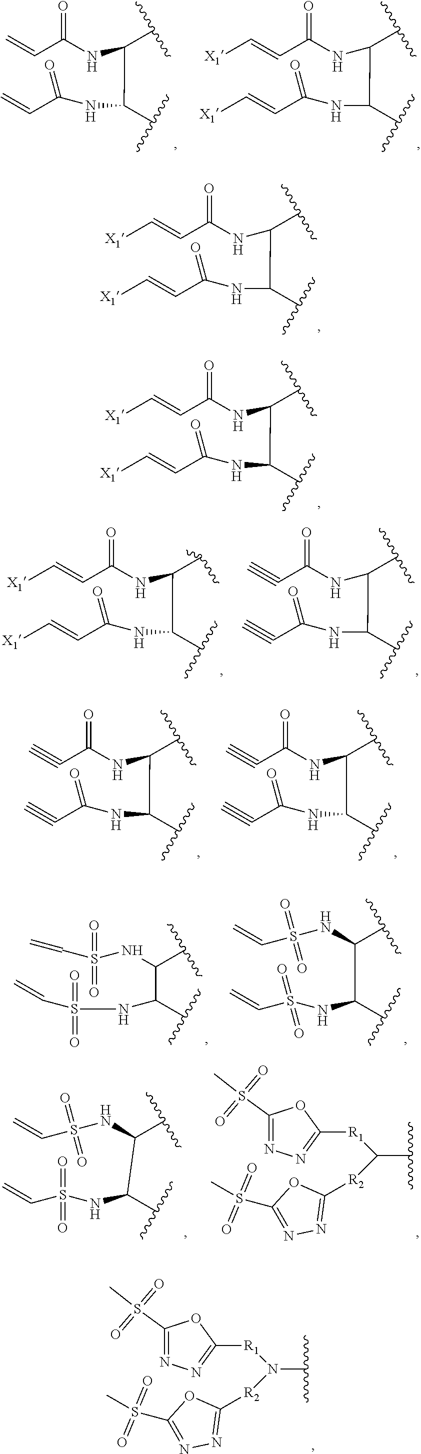

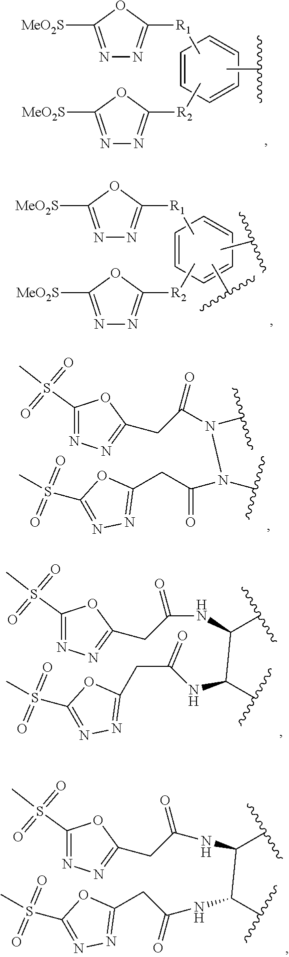

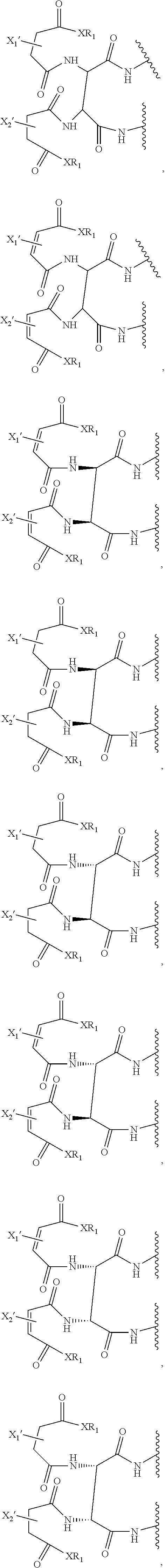

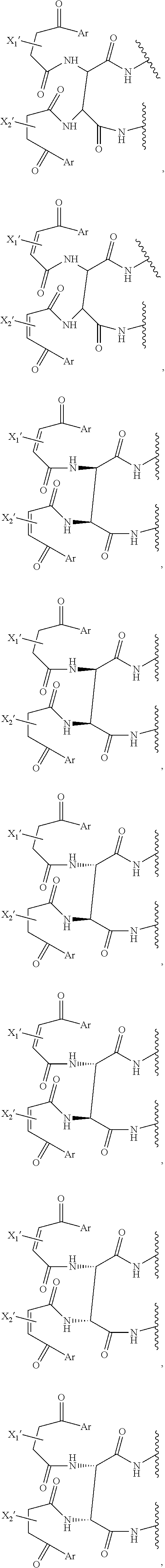

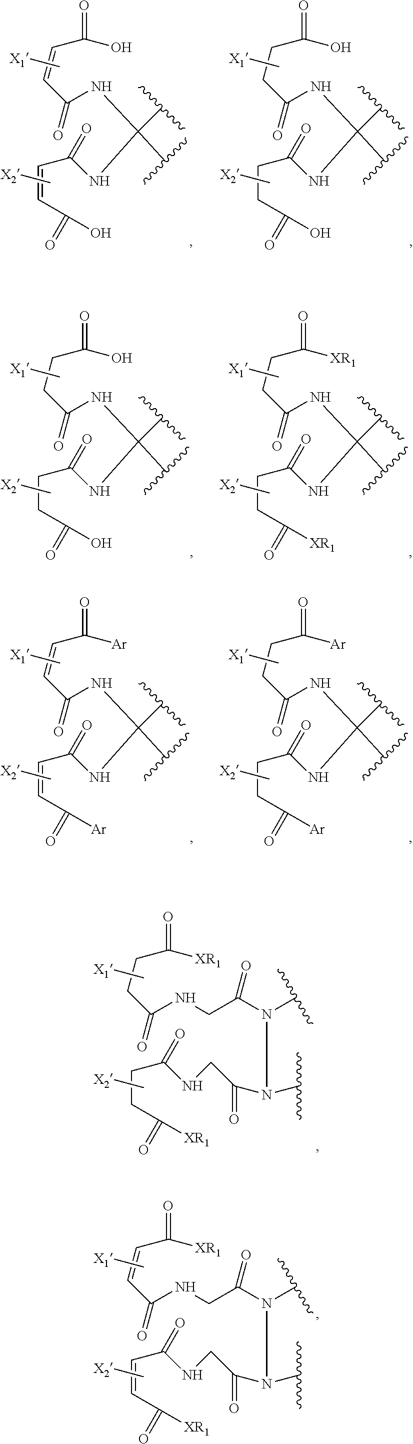

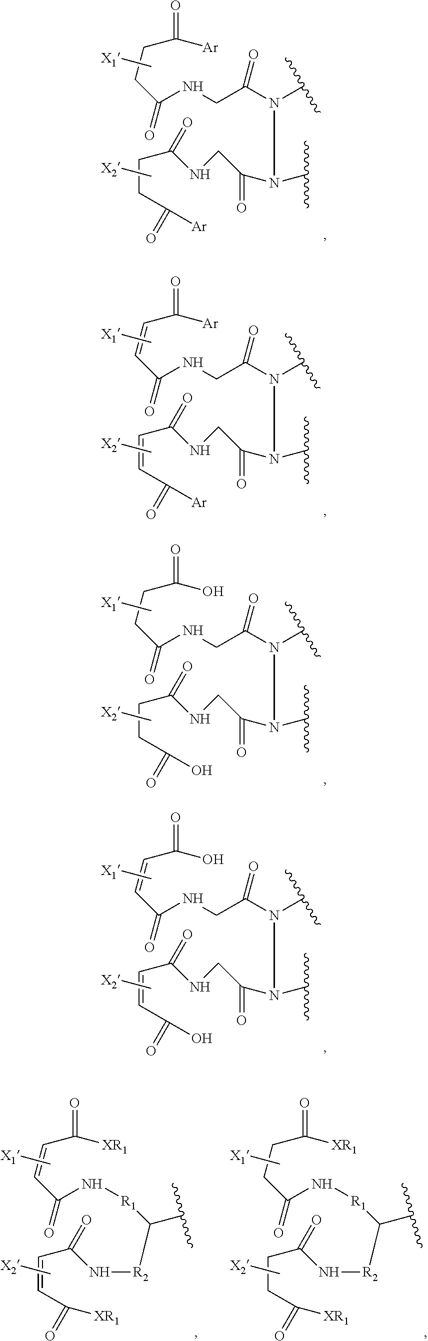

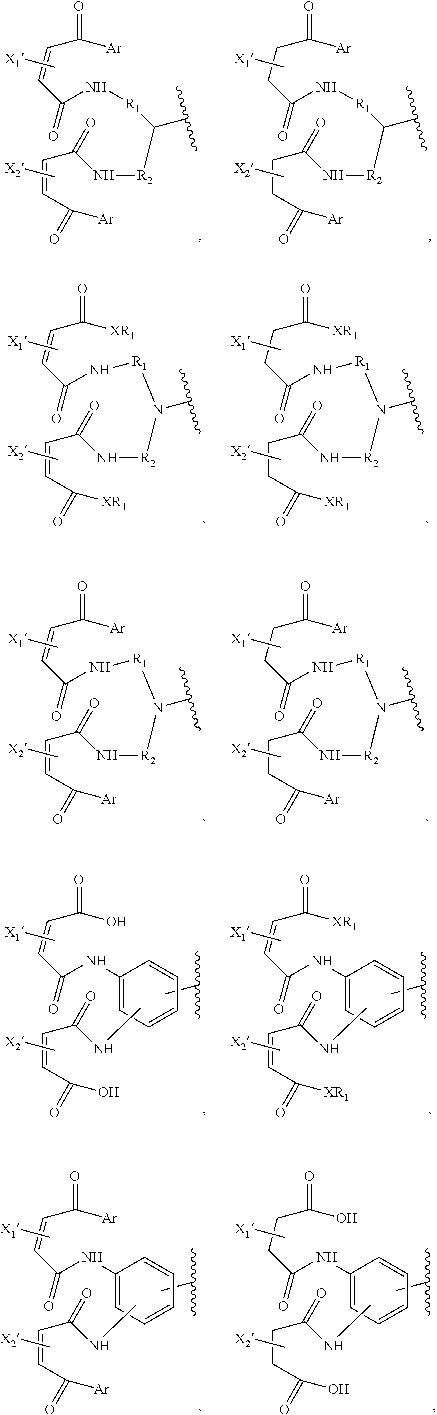

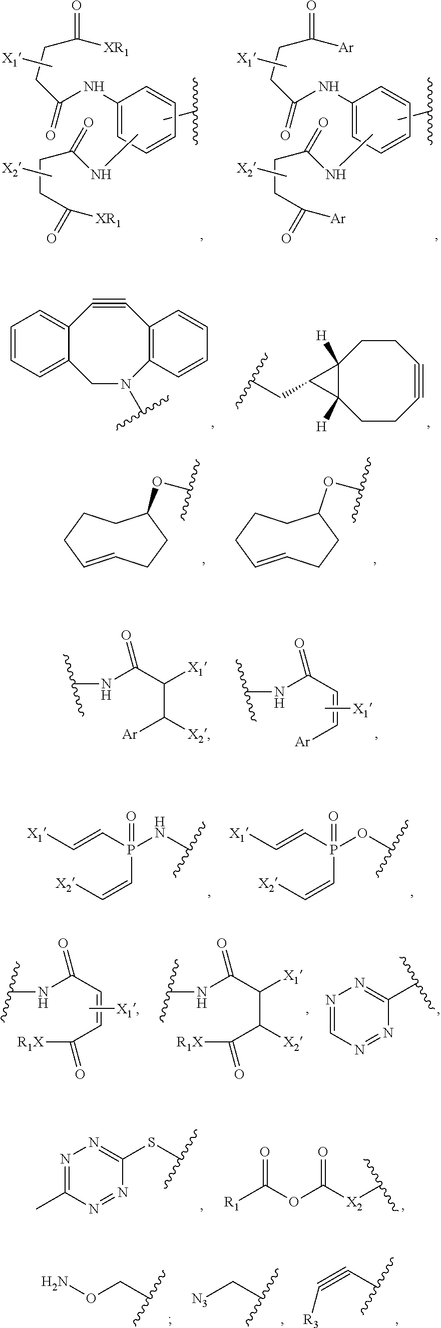

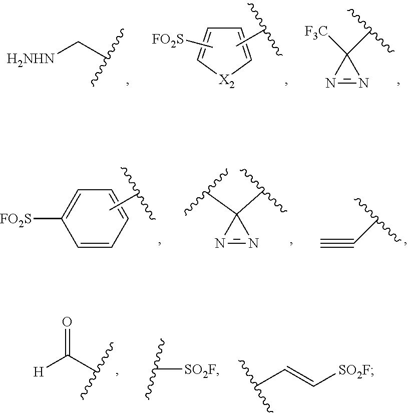

wherein X₁′ and X₂′ are independently F, Cl, Br, I, OTf, OMs, OC₆H₄ (NO₂), OC₆H₃ (NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR¹, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions;

In the formula (VI) and formula (VII) wherein

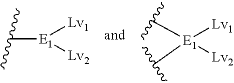

can be accordingly selected from:

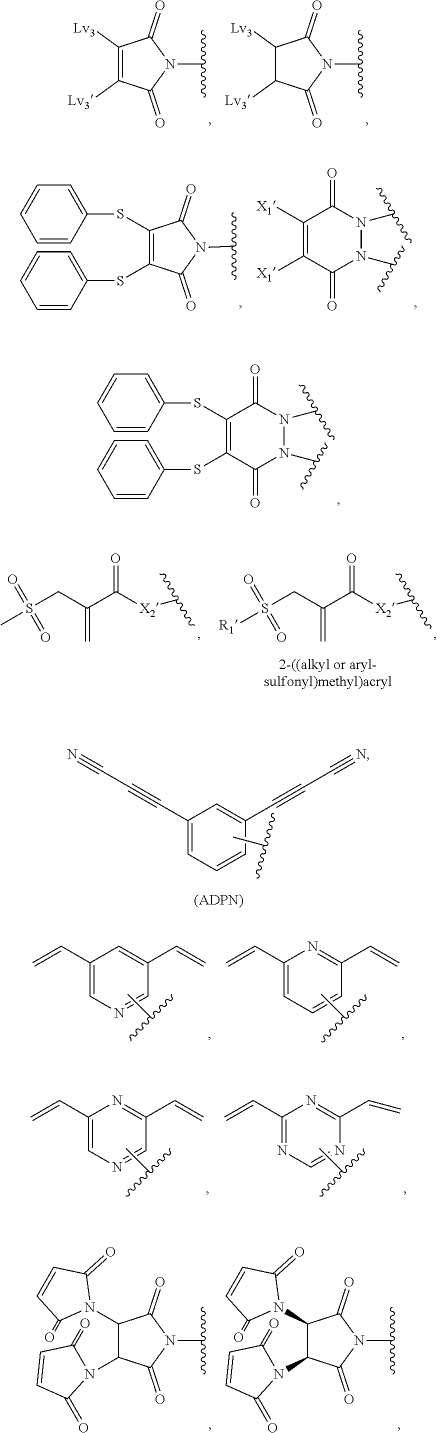

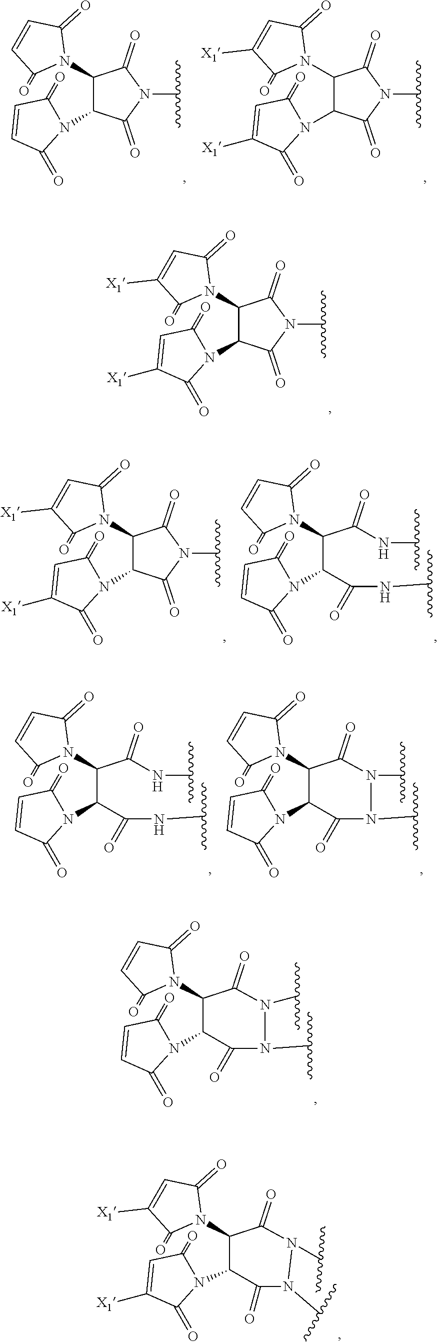

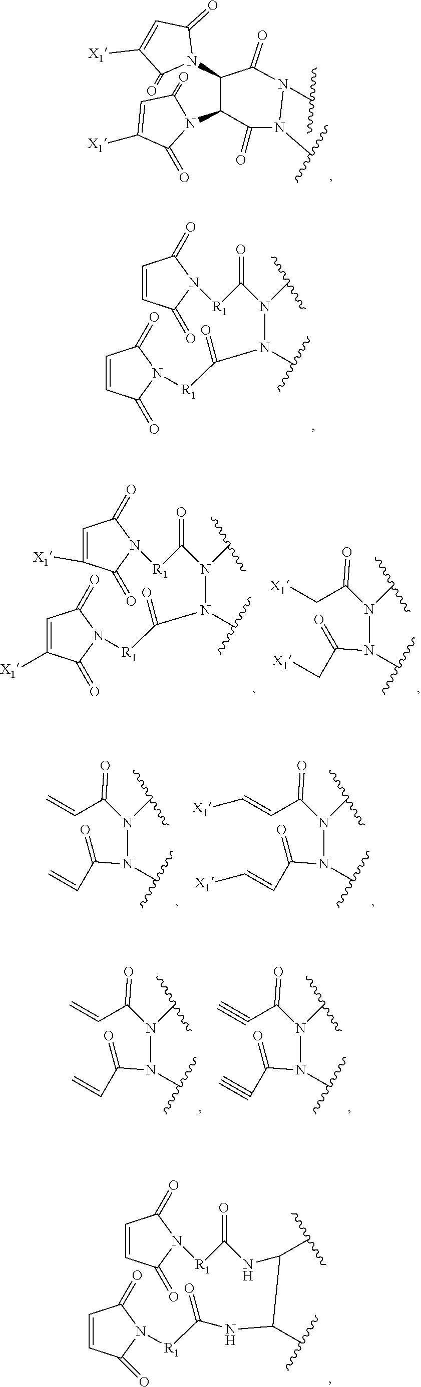

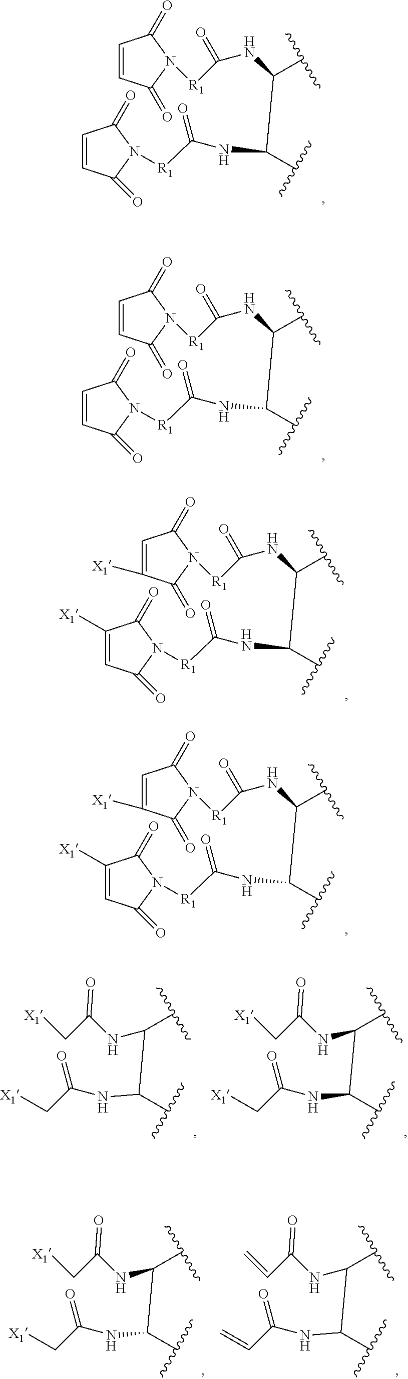

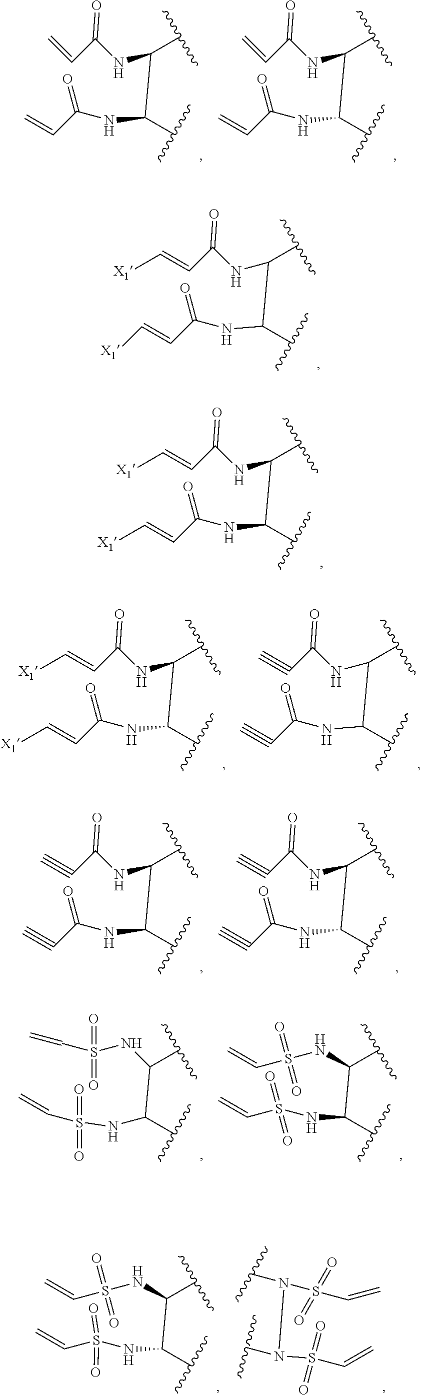

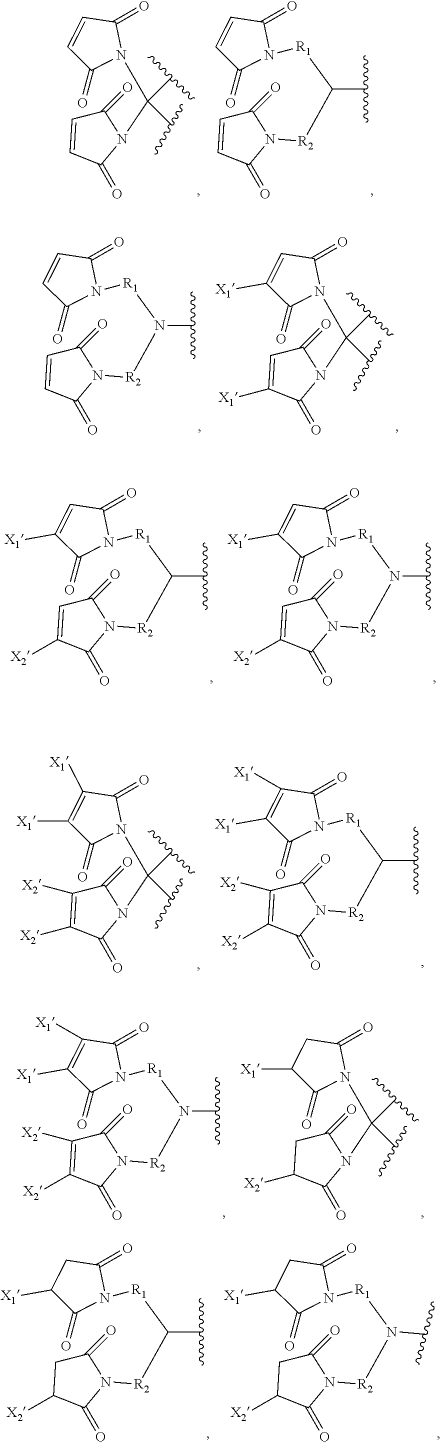

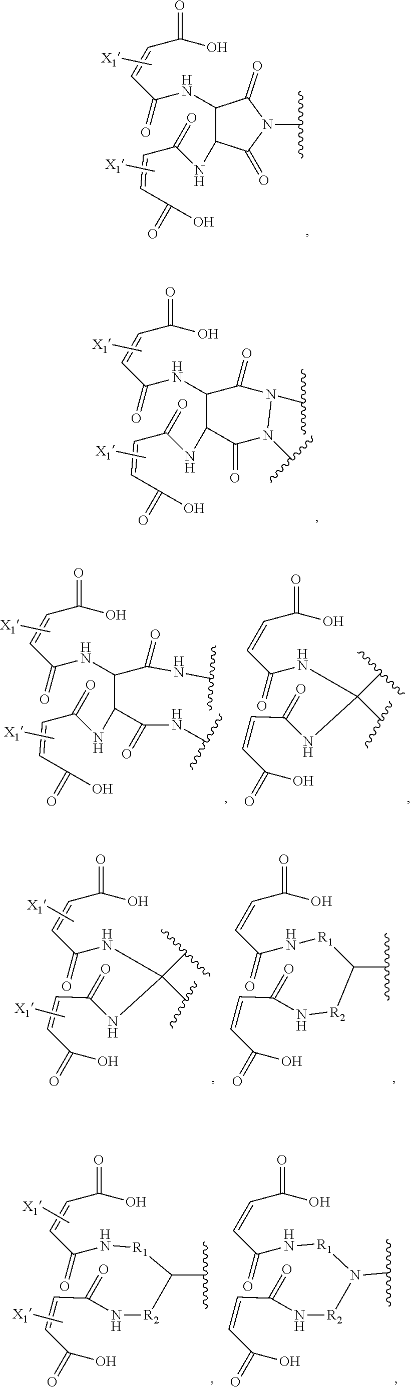

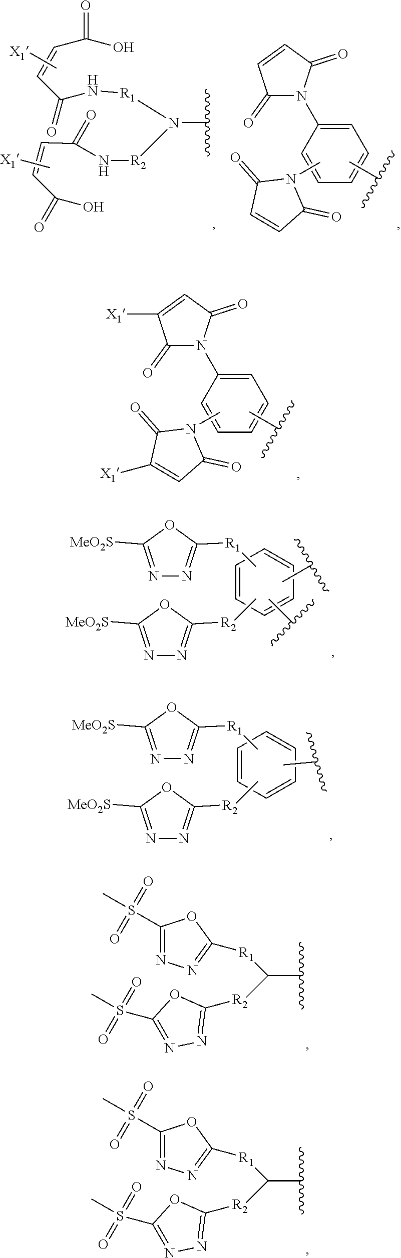

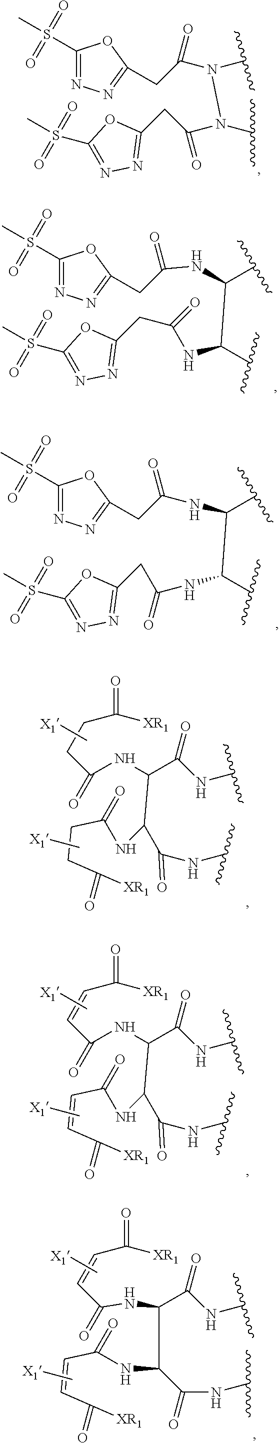

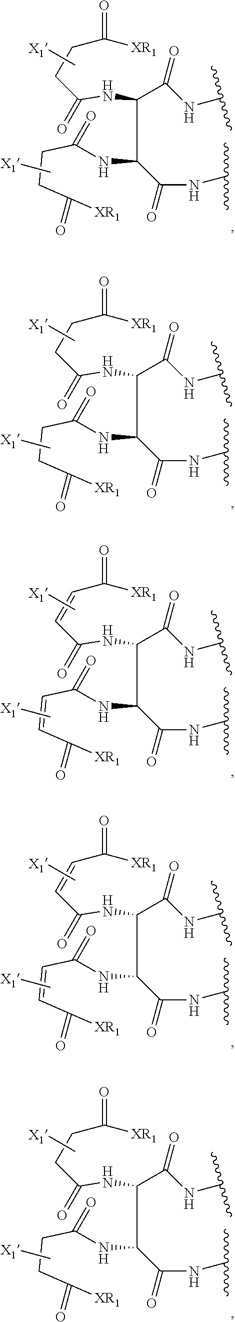

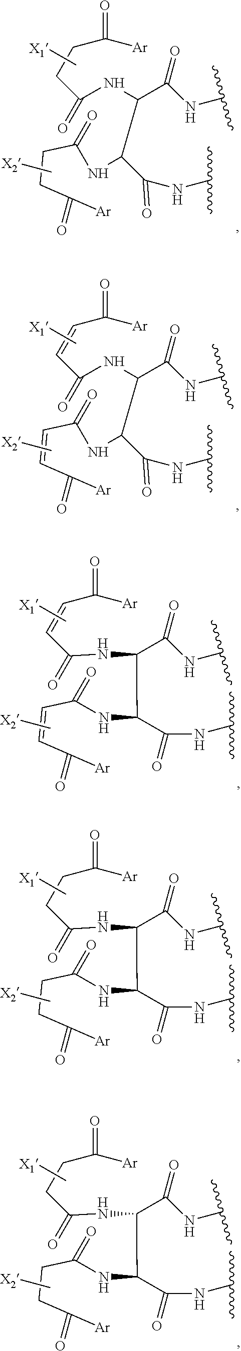

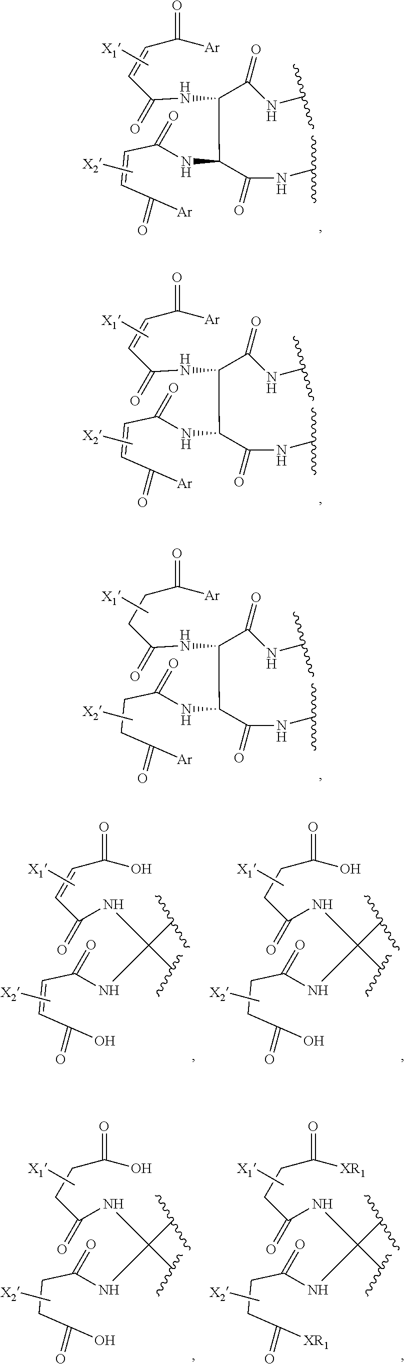

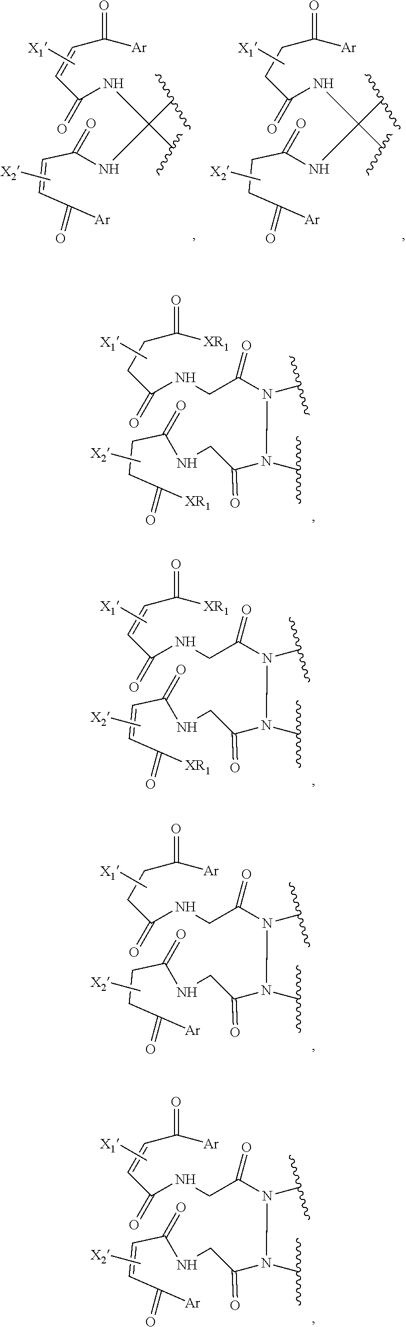

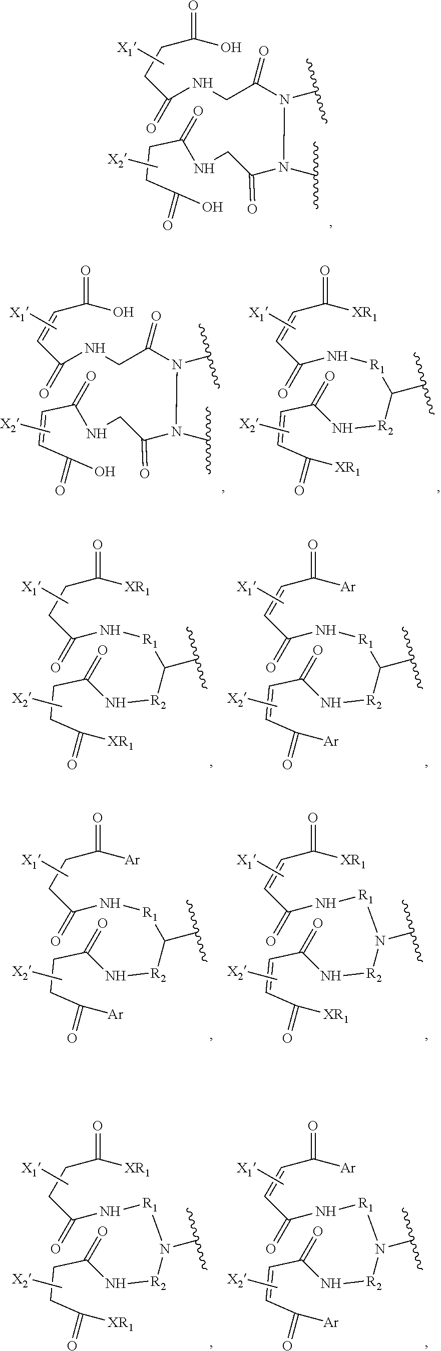

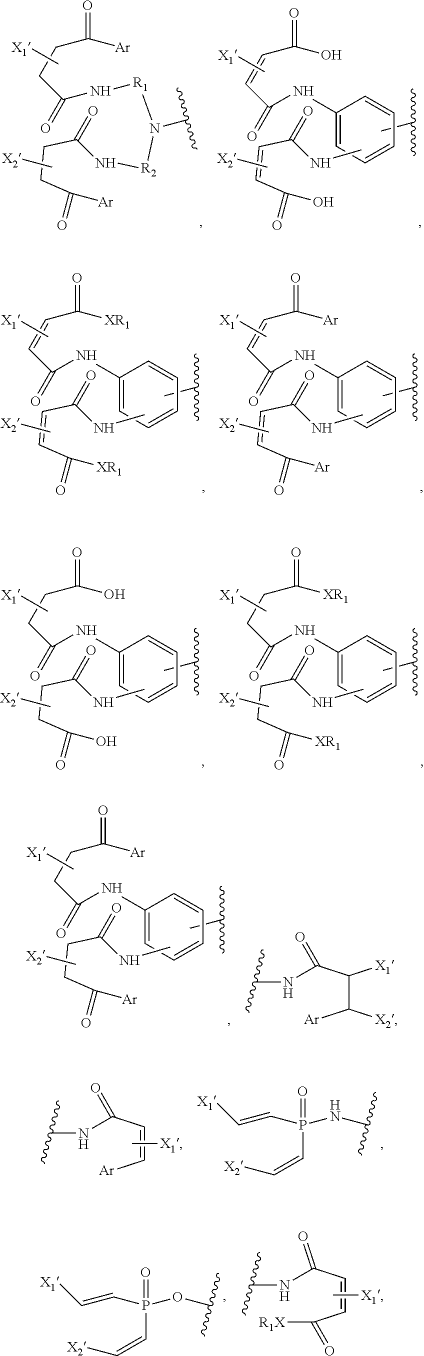

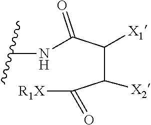

wherein Lv₃, Lv_{3′,} X₁′, and X₂′, are described above; the connecting bond “—” in the middle of the two atoms means it can link either one of the two atoms.

Examples of Formula (V), (VI), (VII) and (V′) are illustrated below:

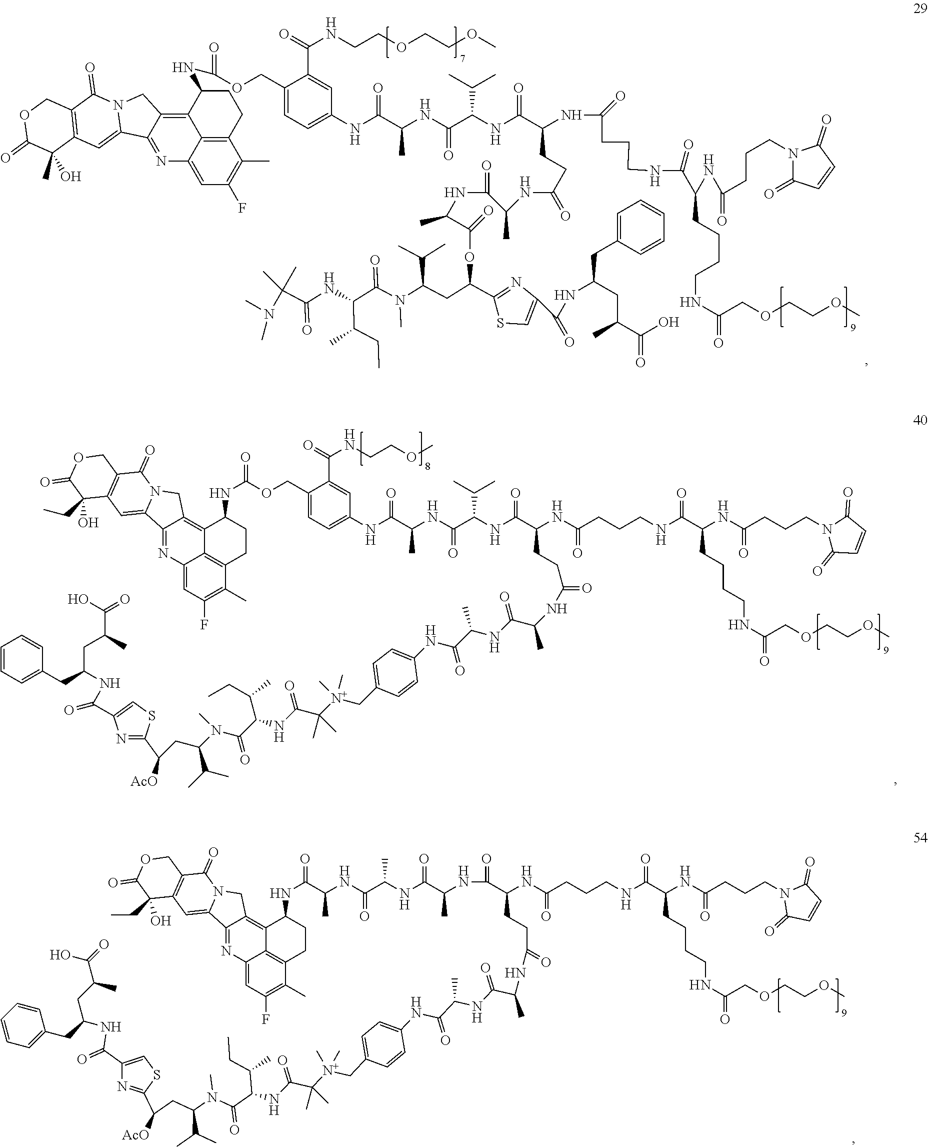

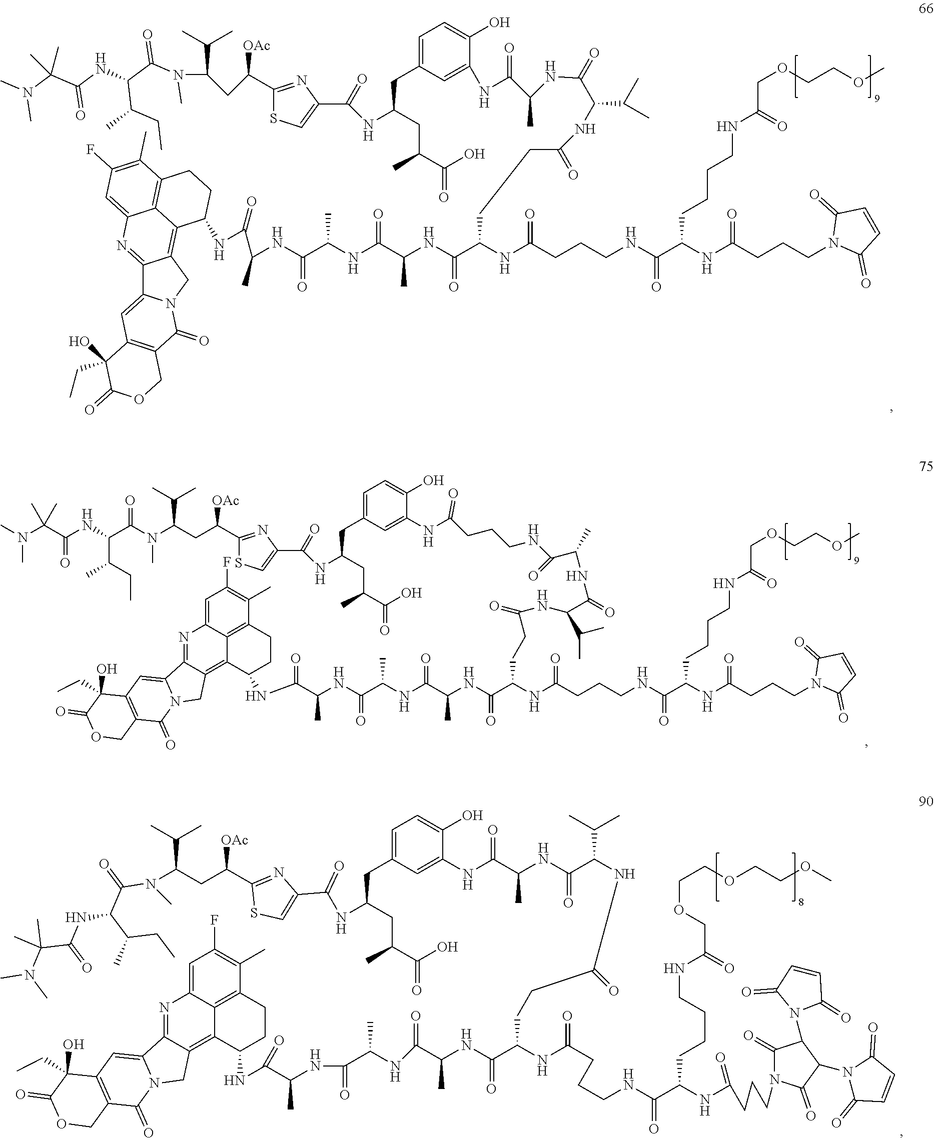

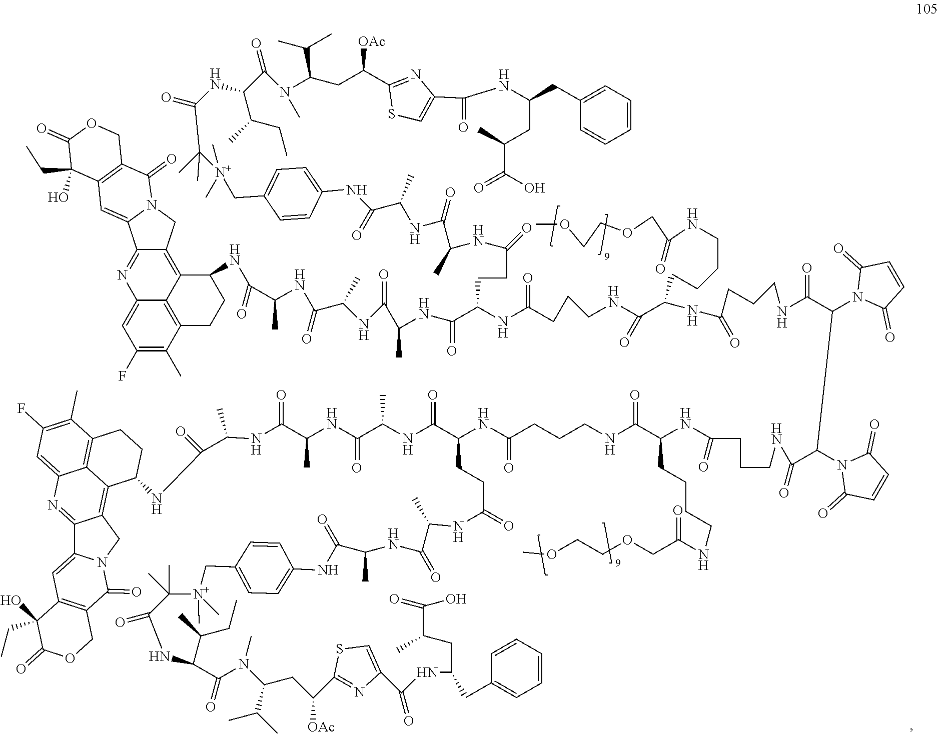

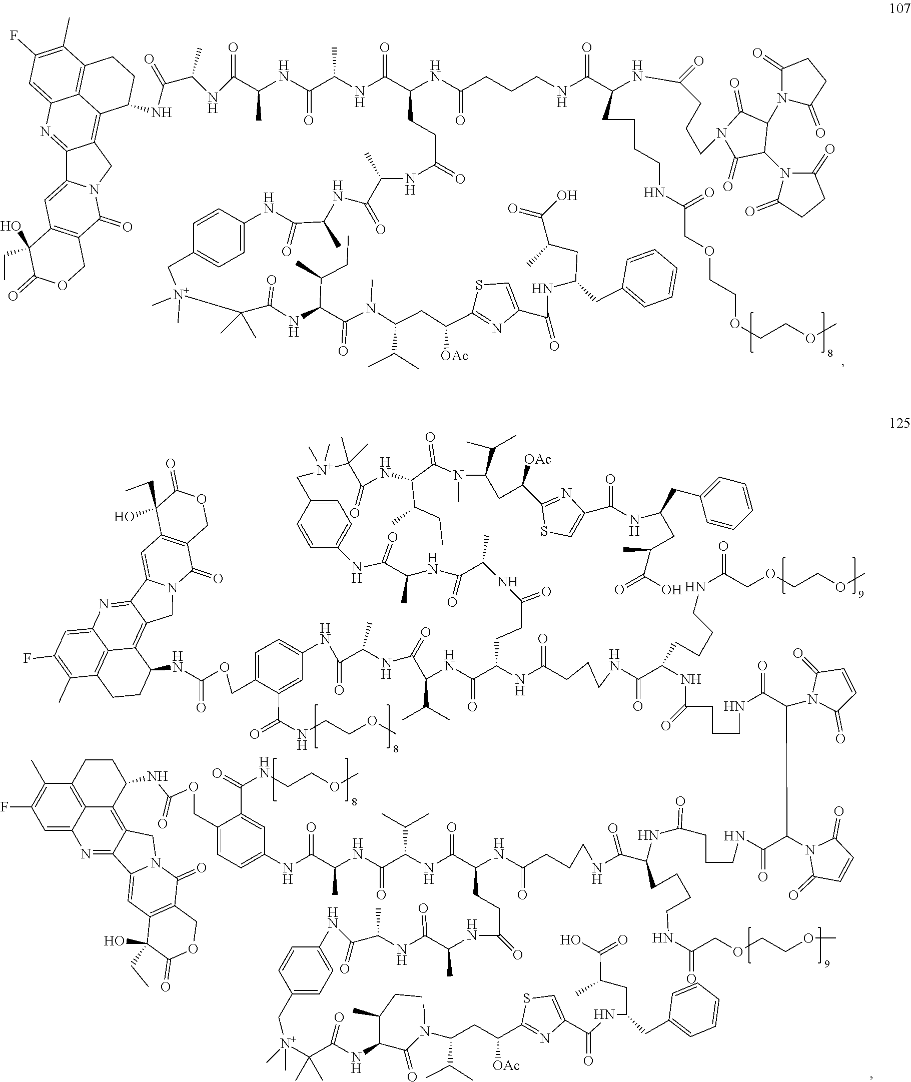

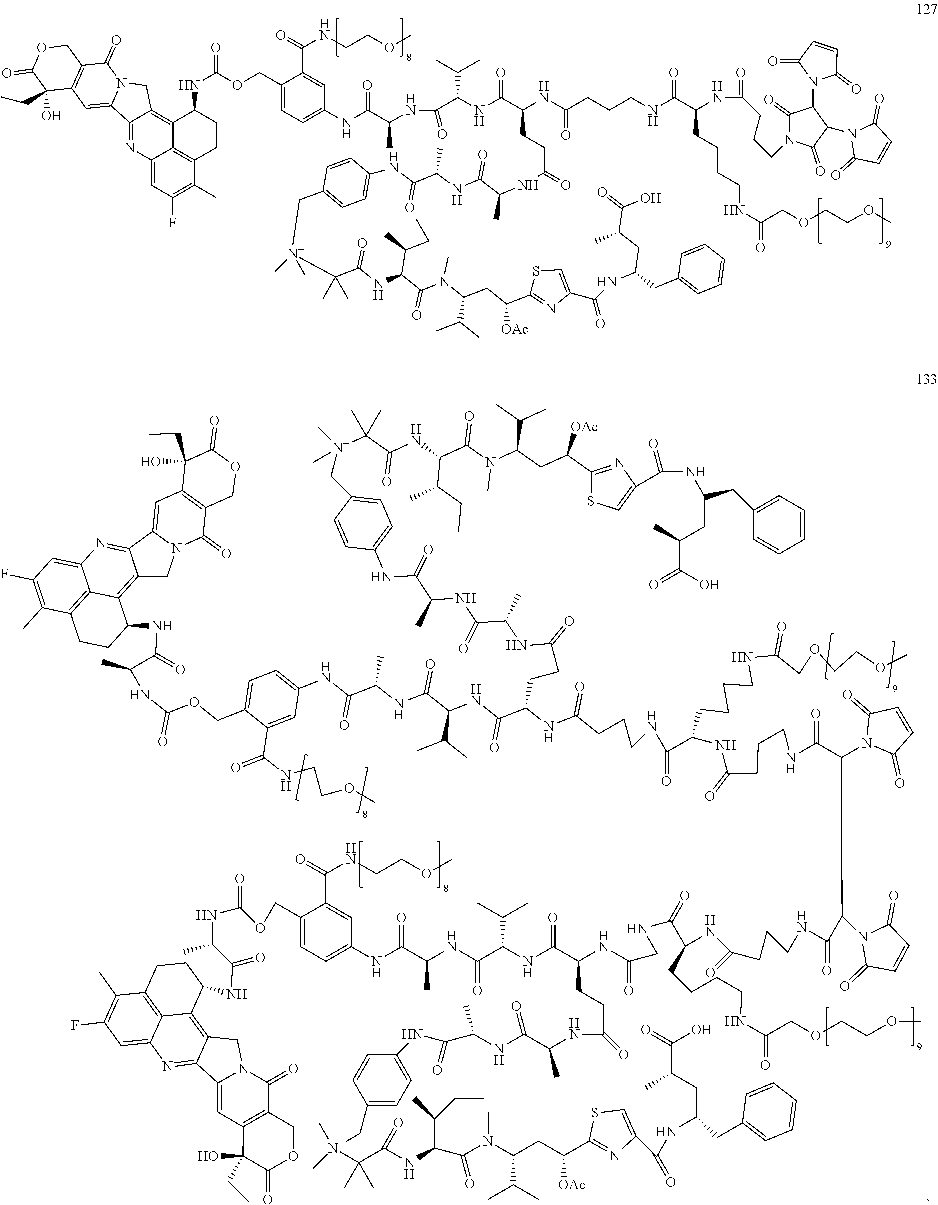

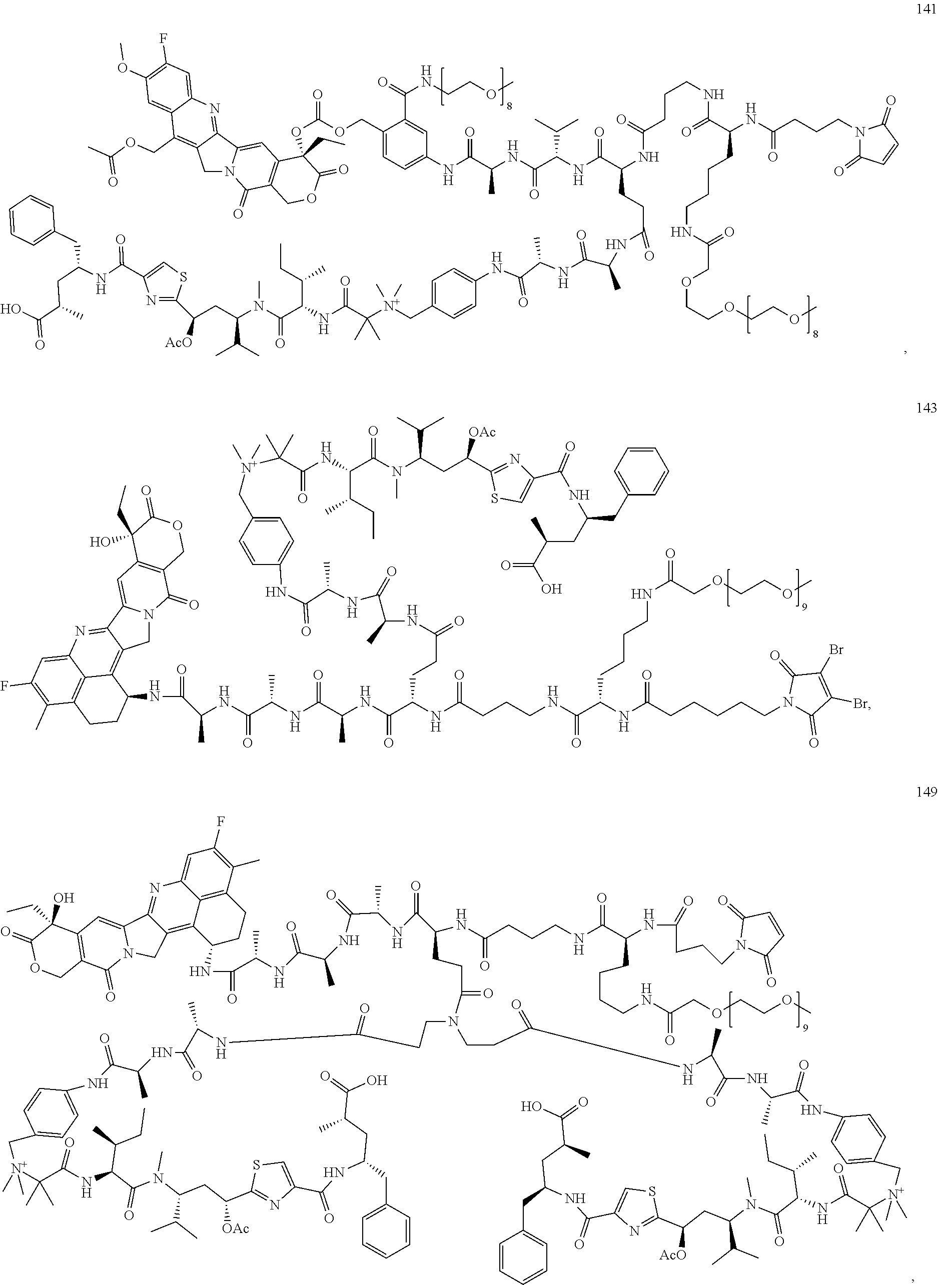

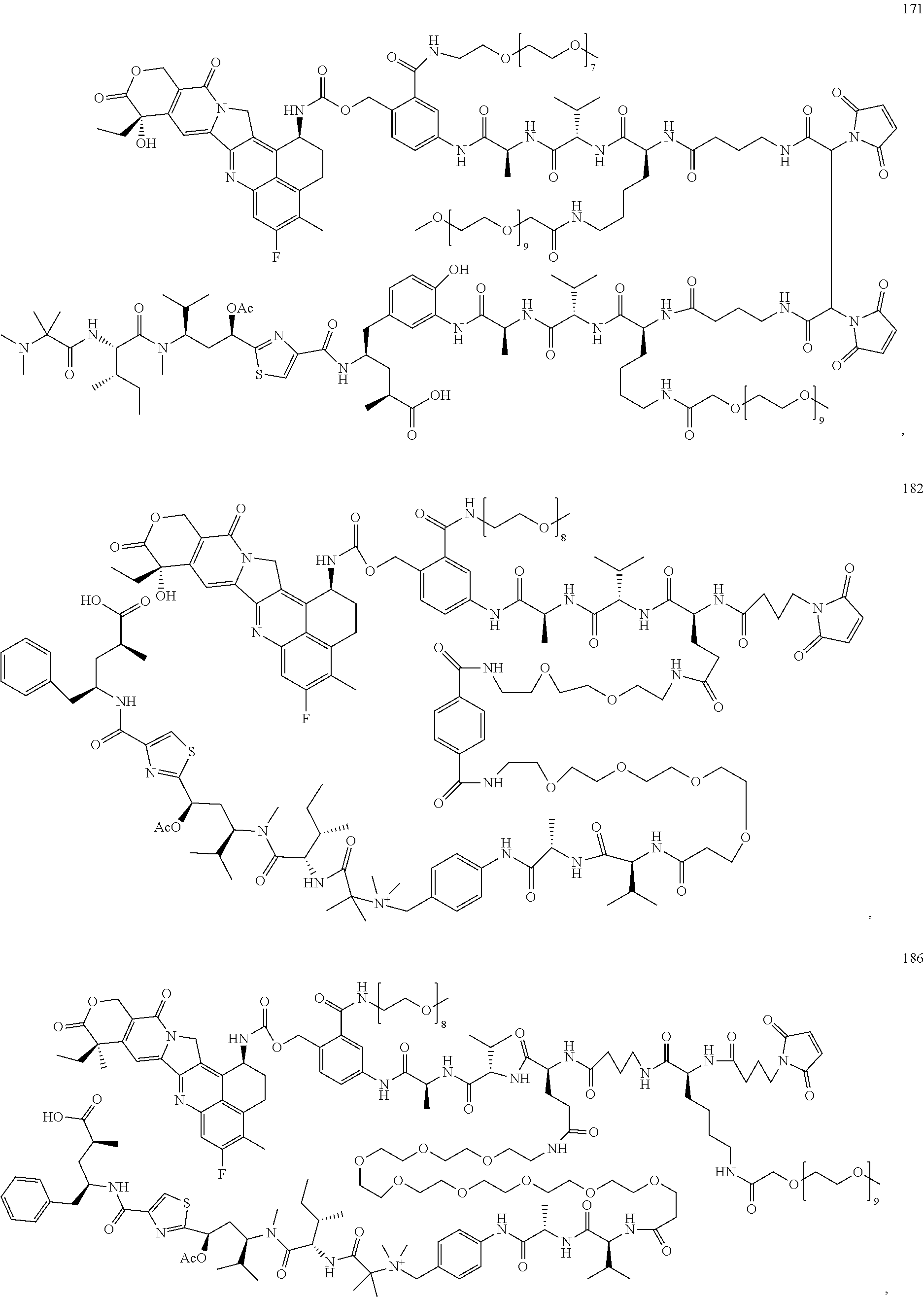

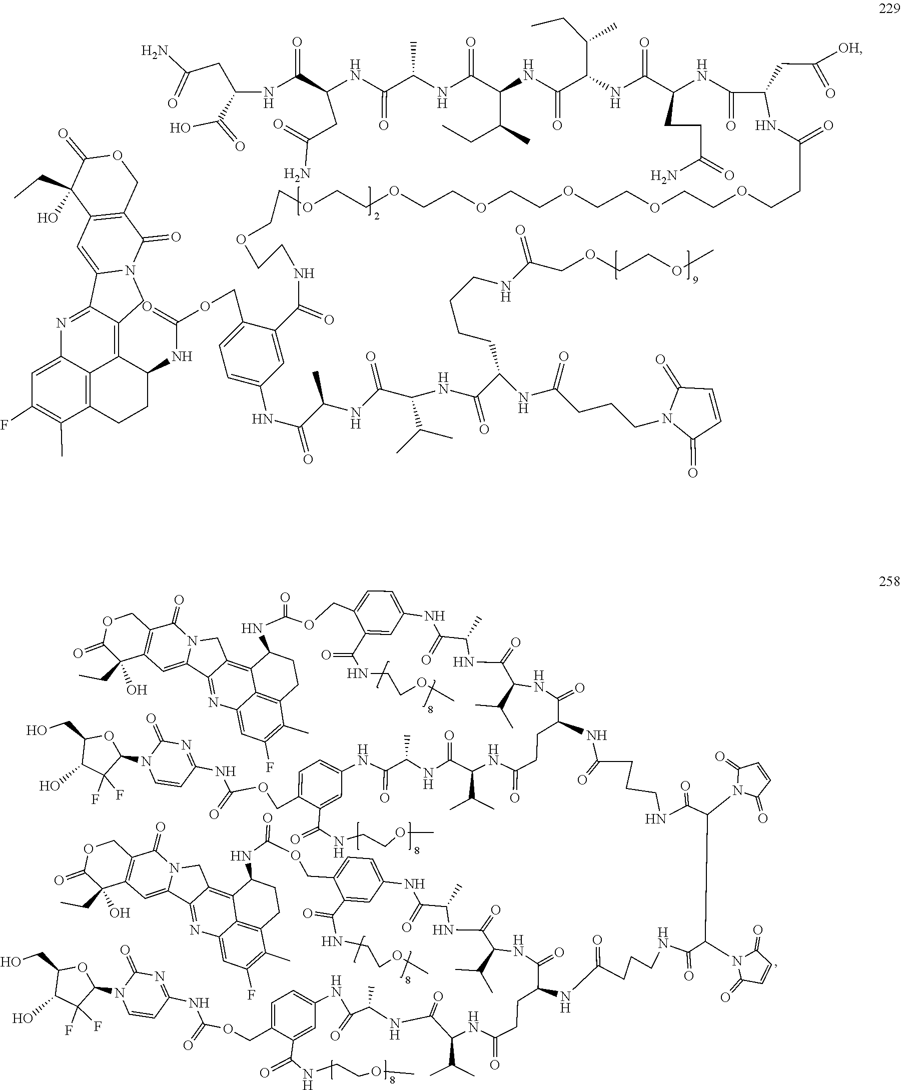

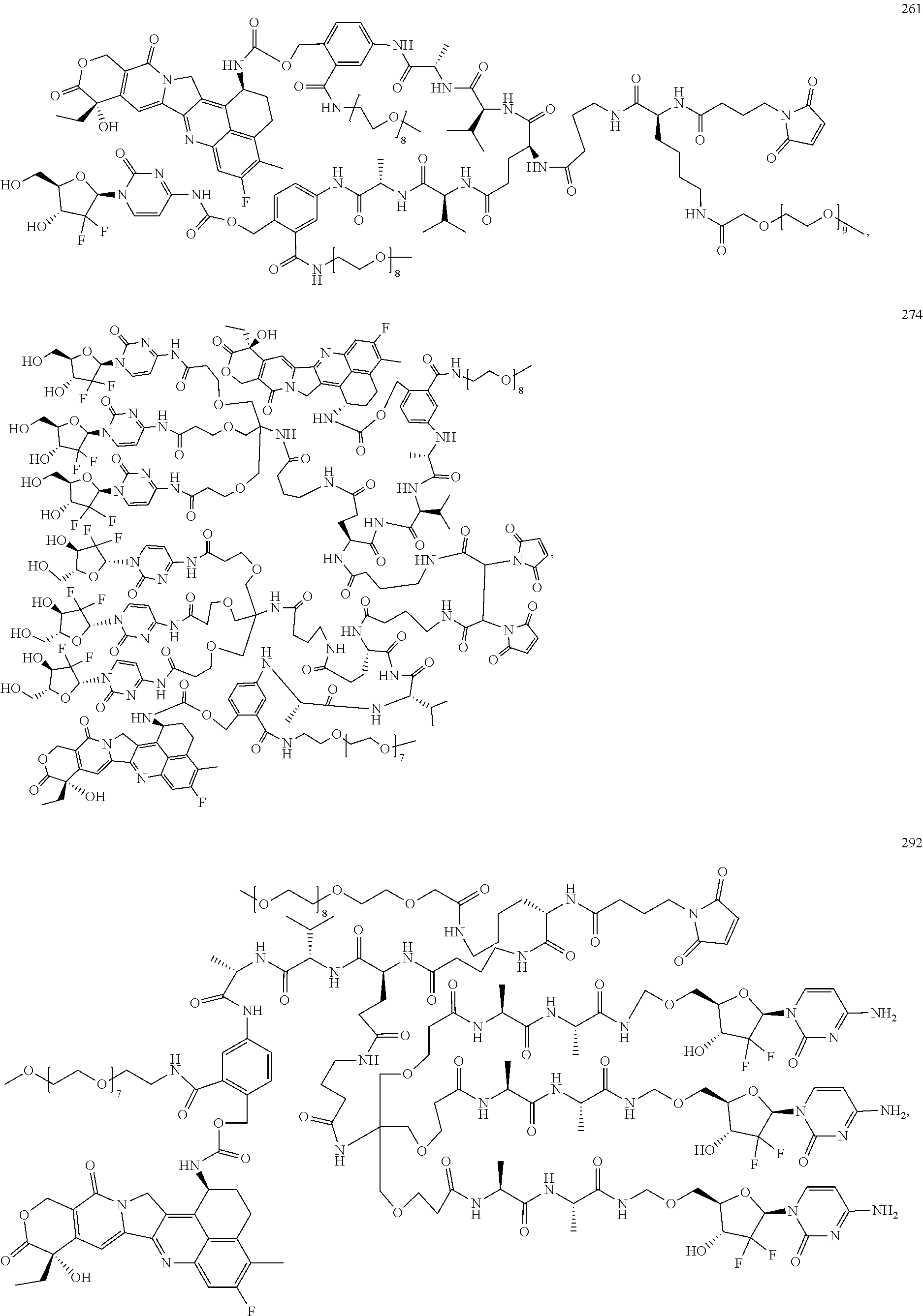

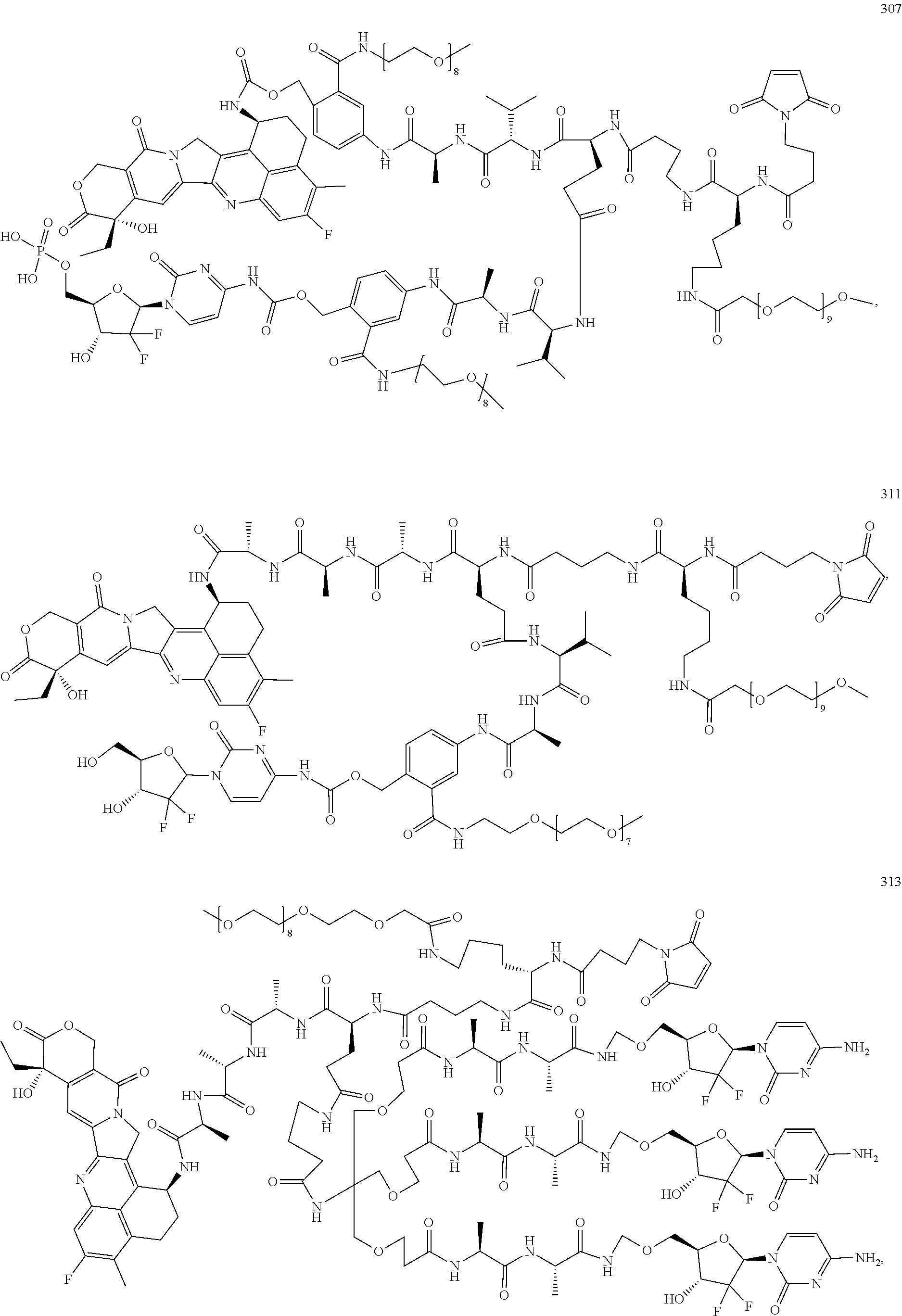

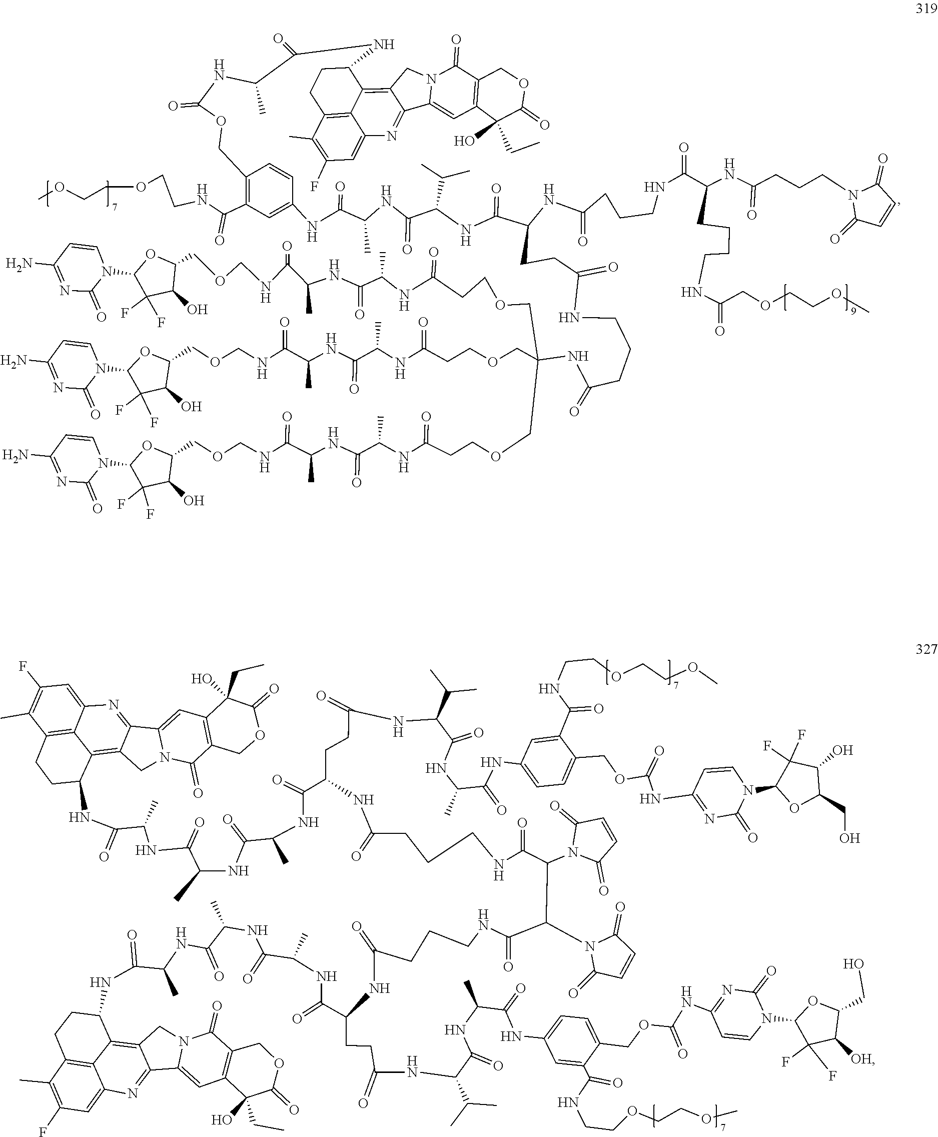

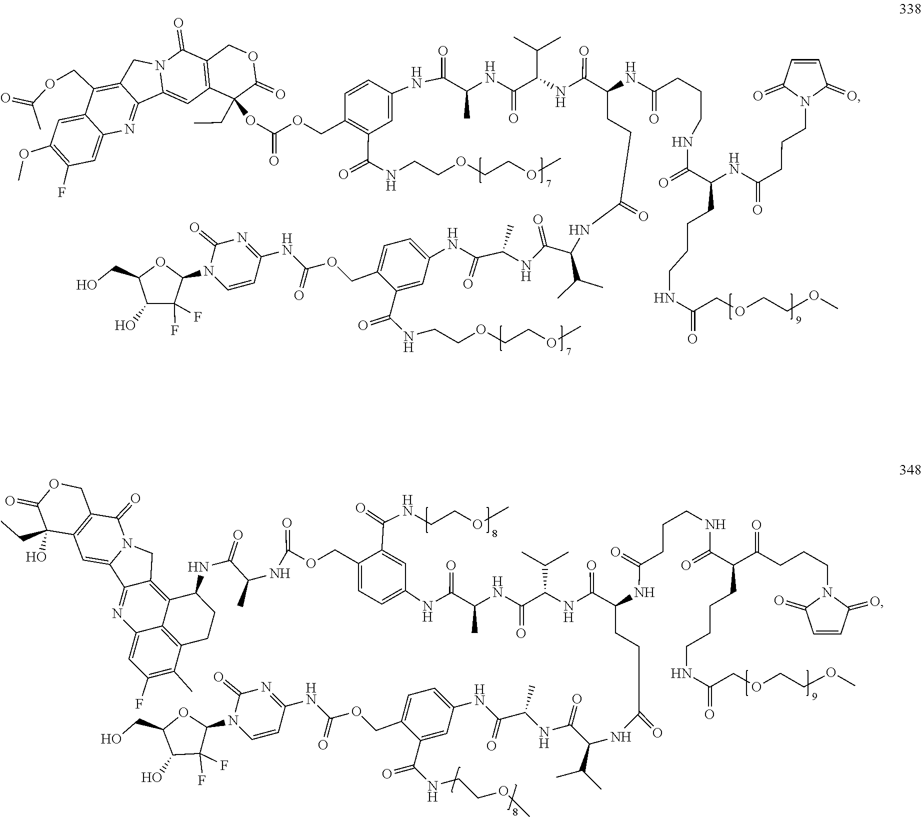

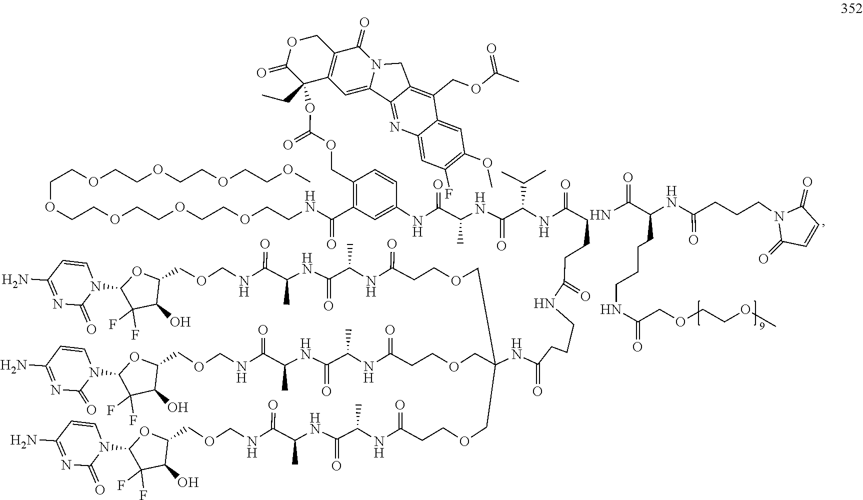

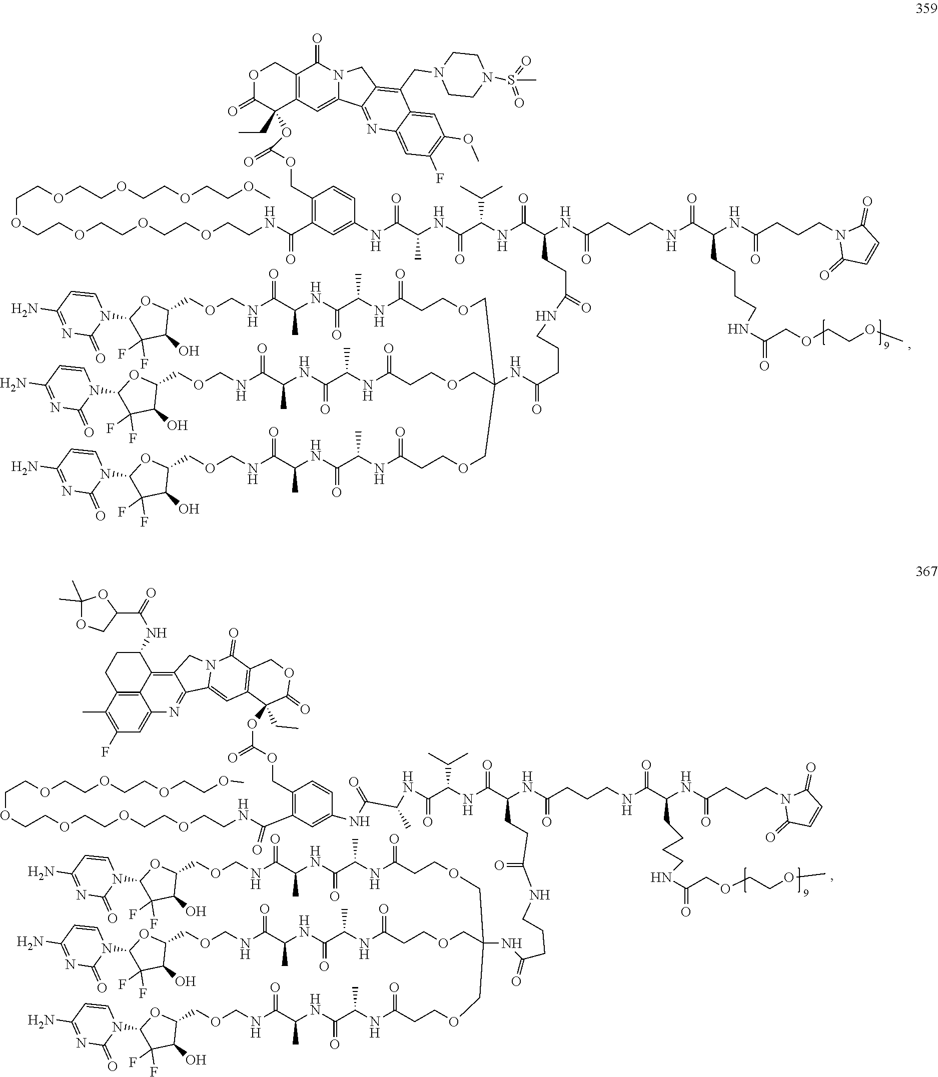

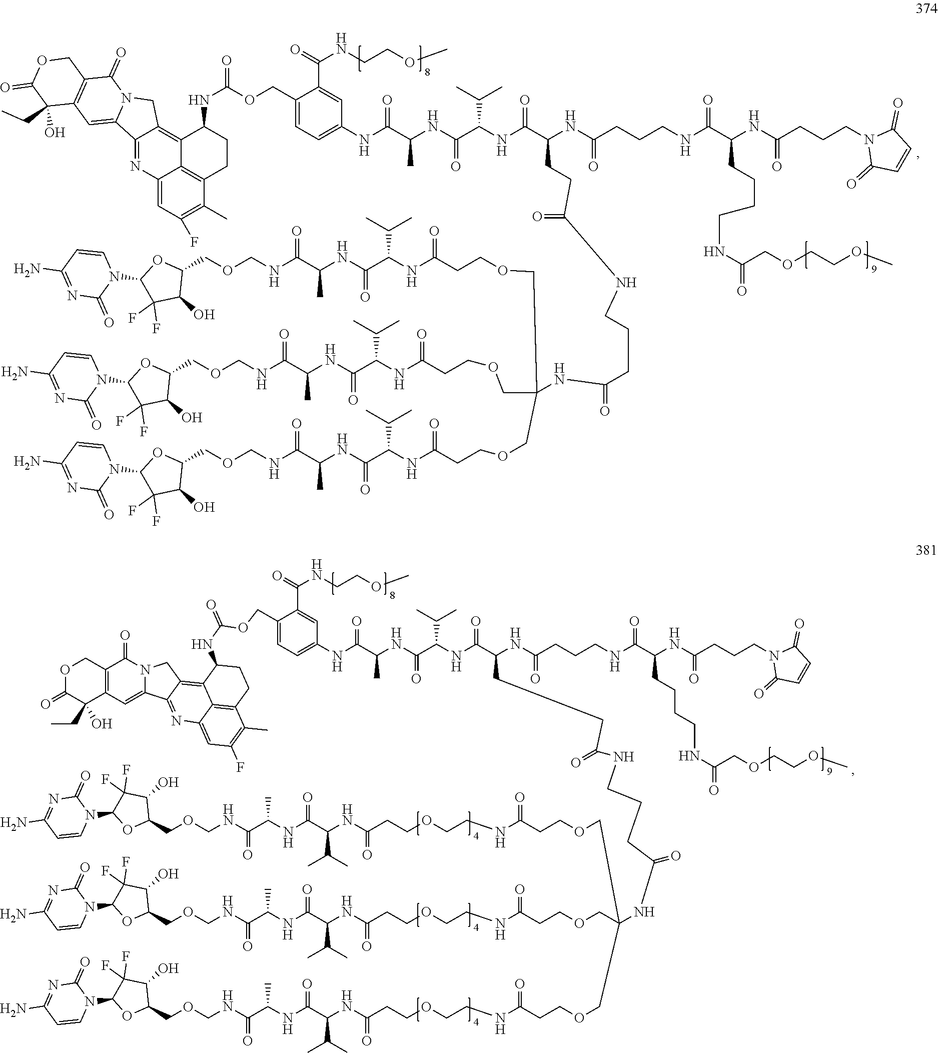

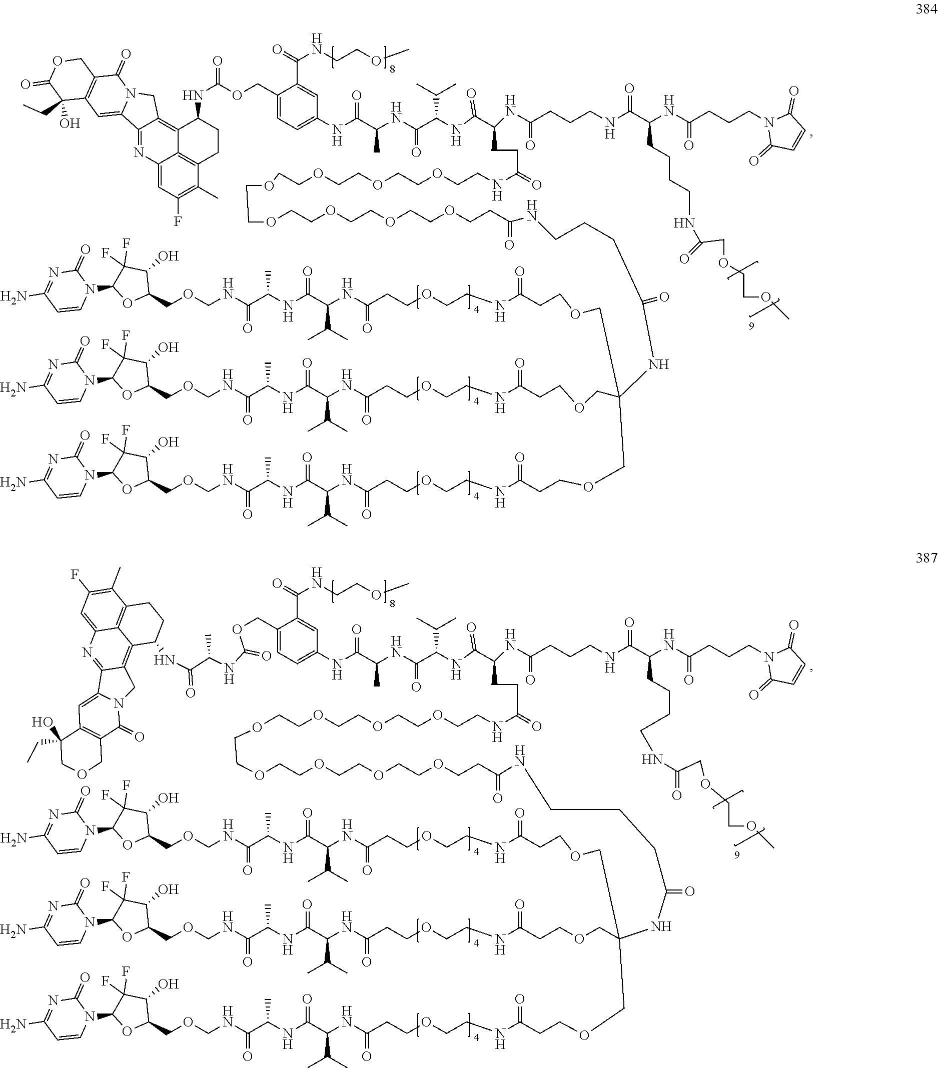

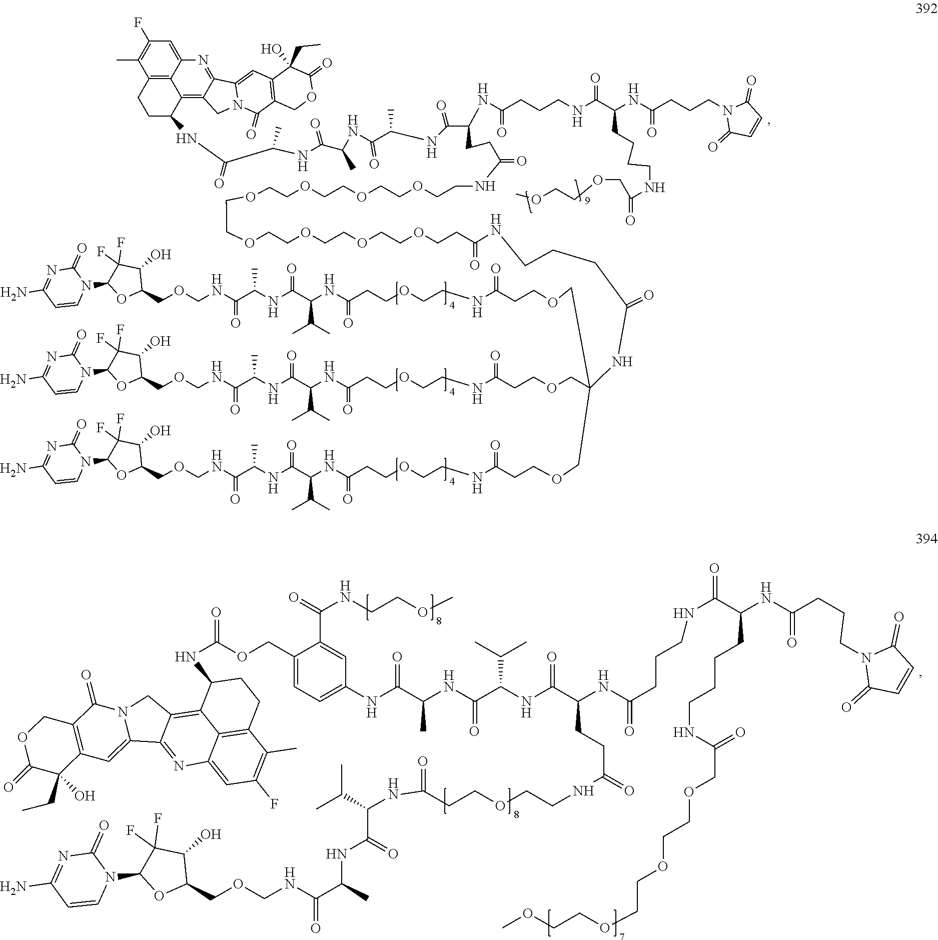

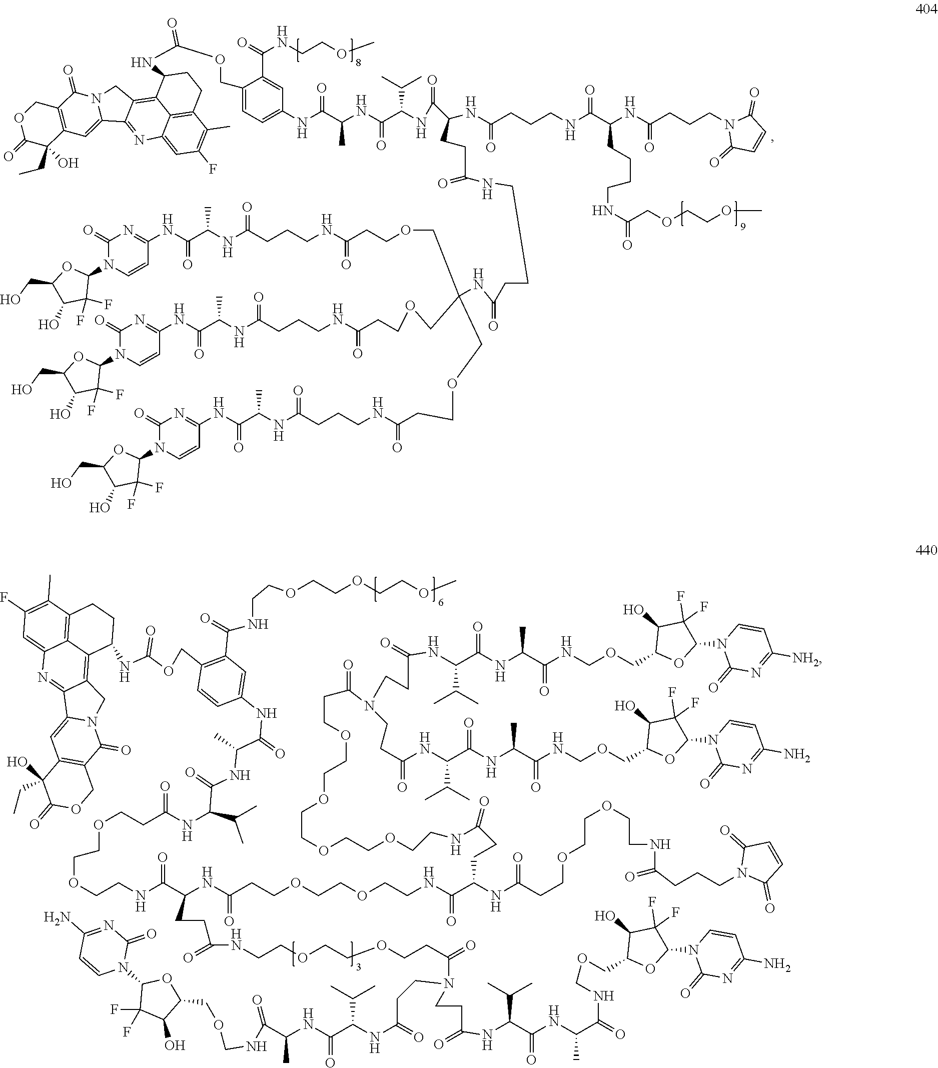

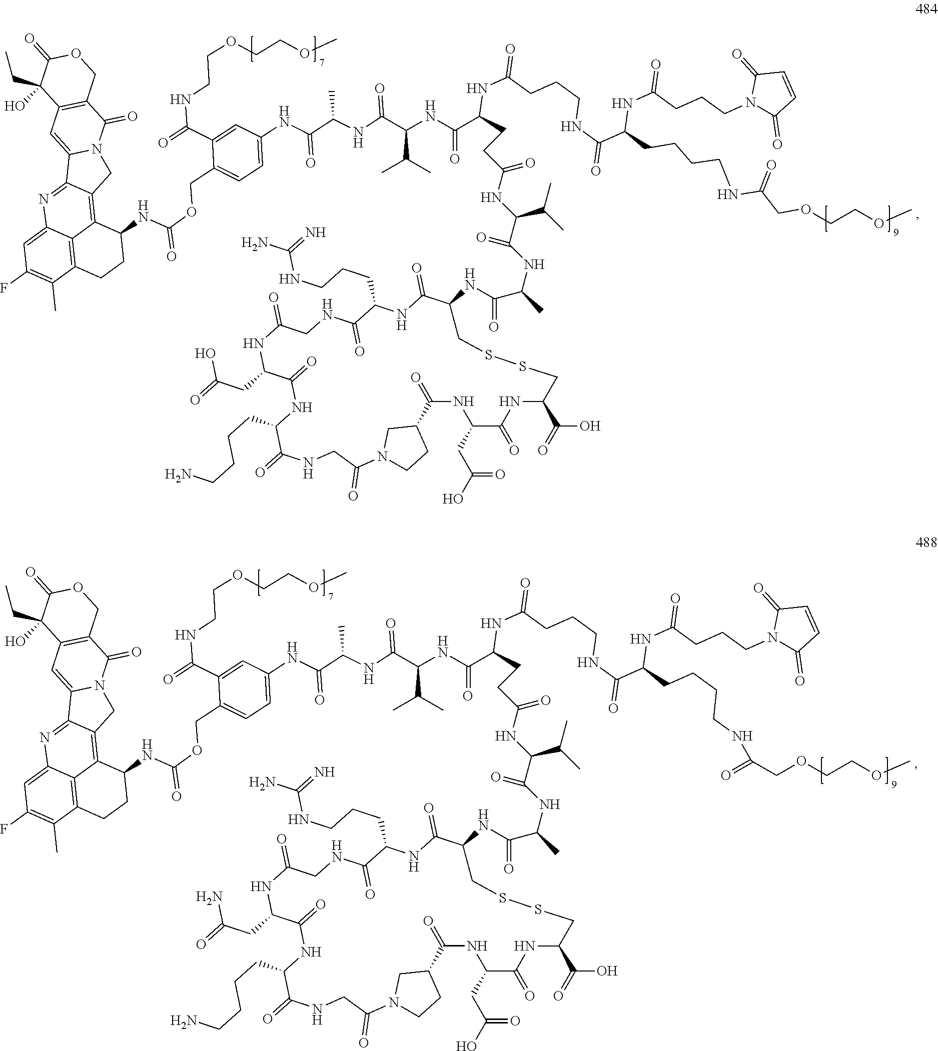

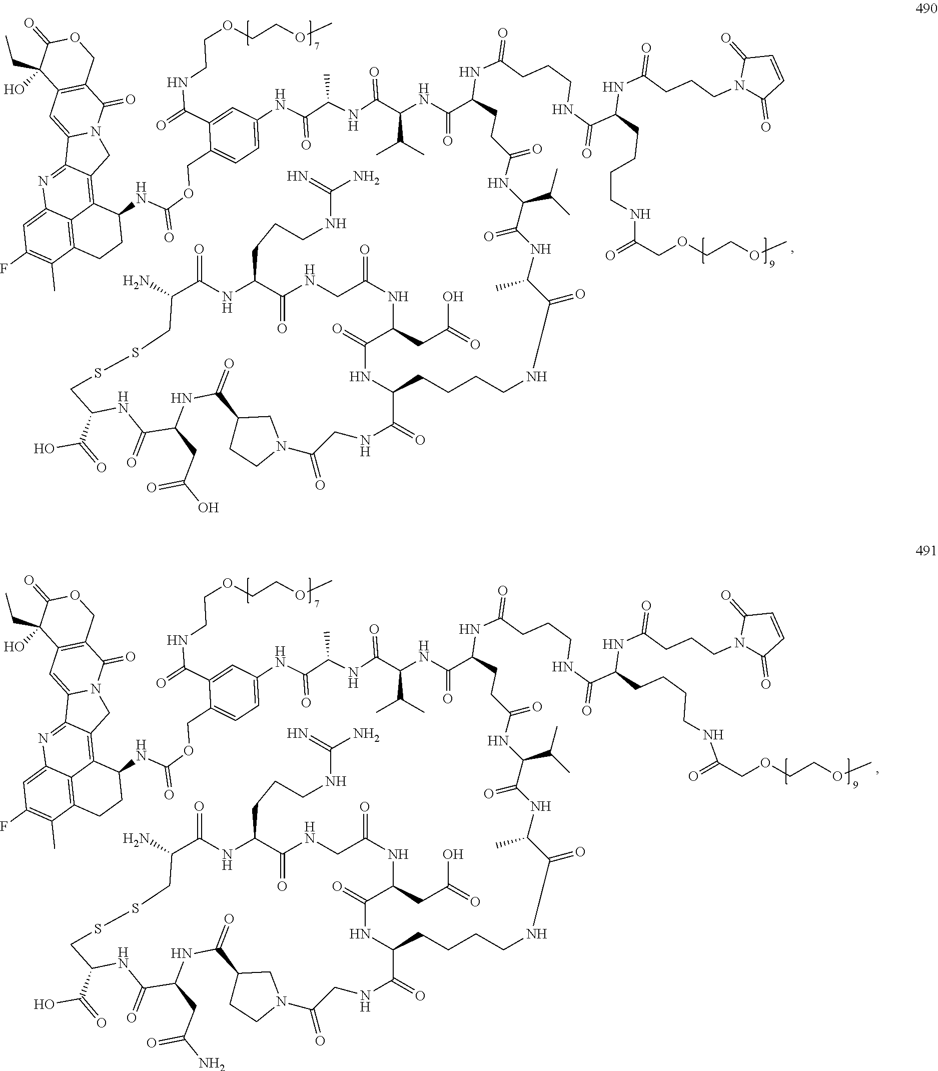

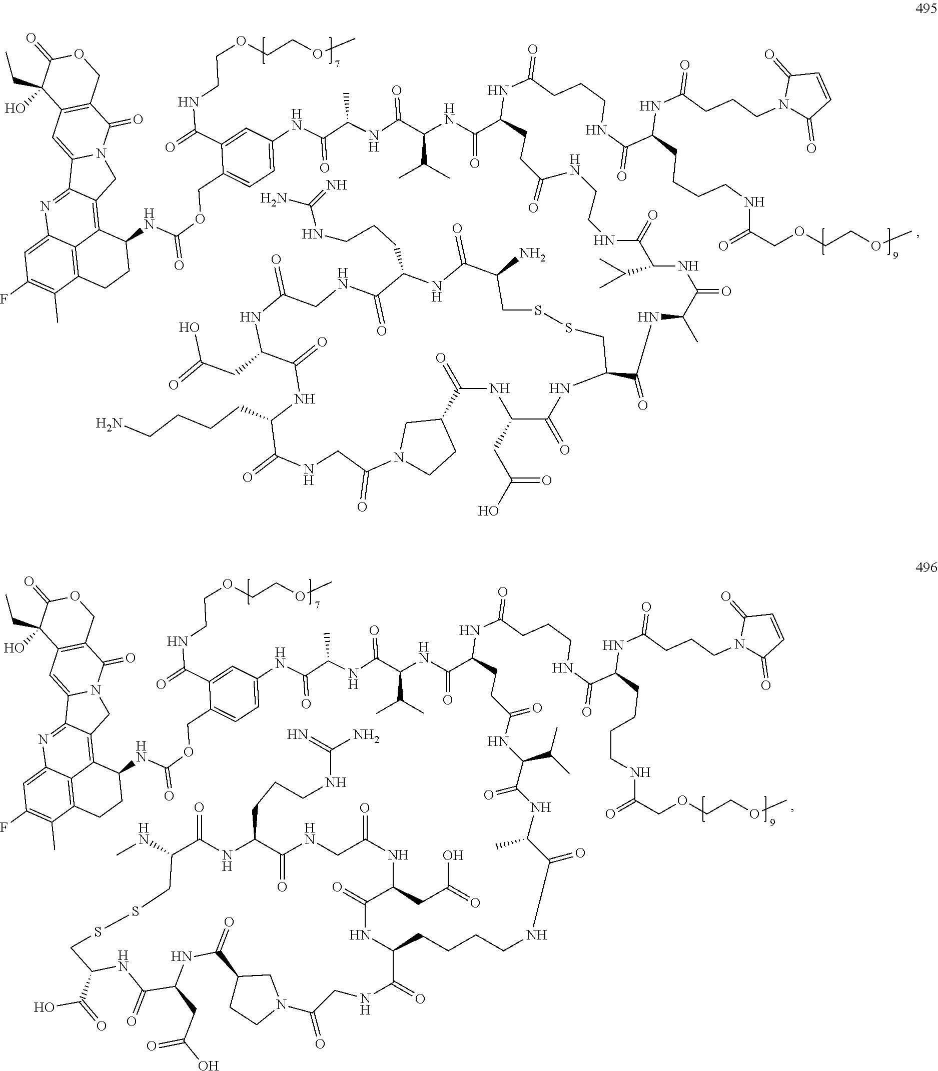

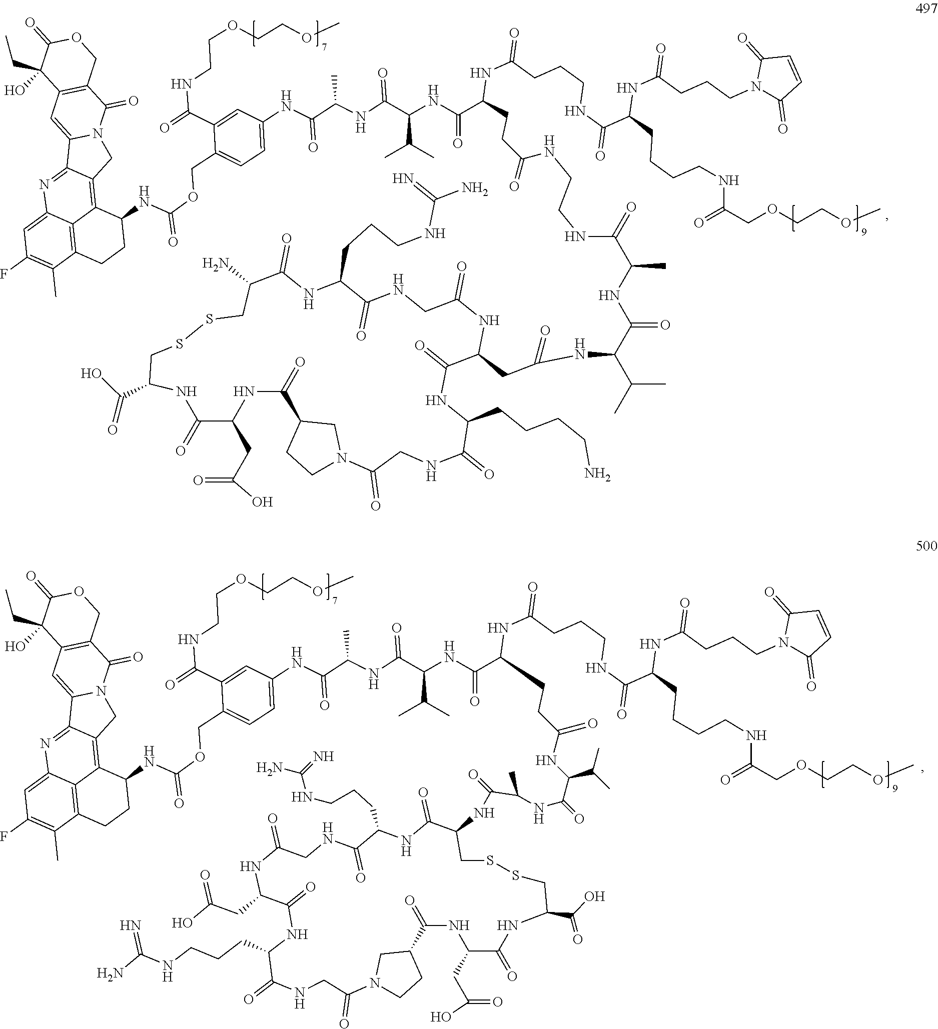

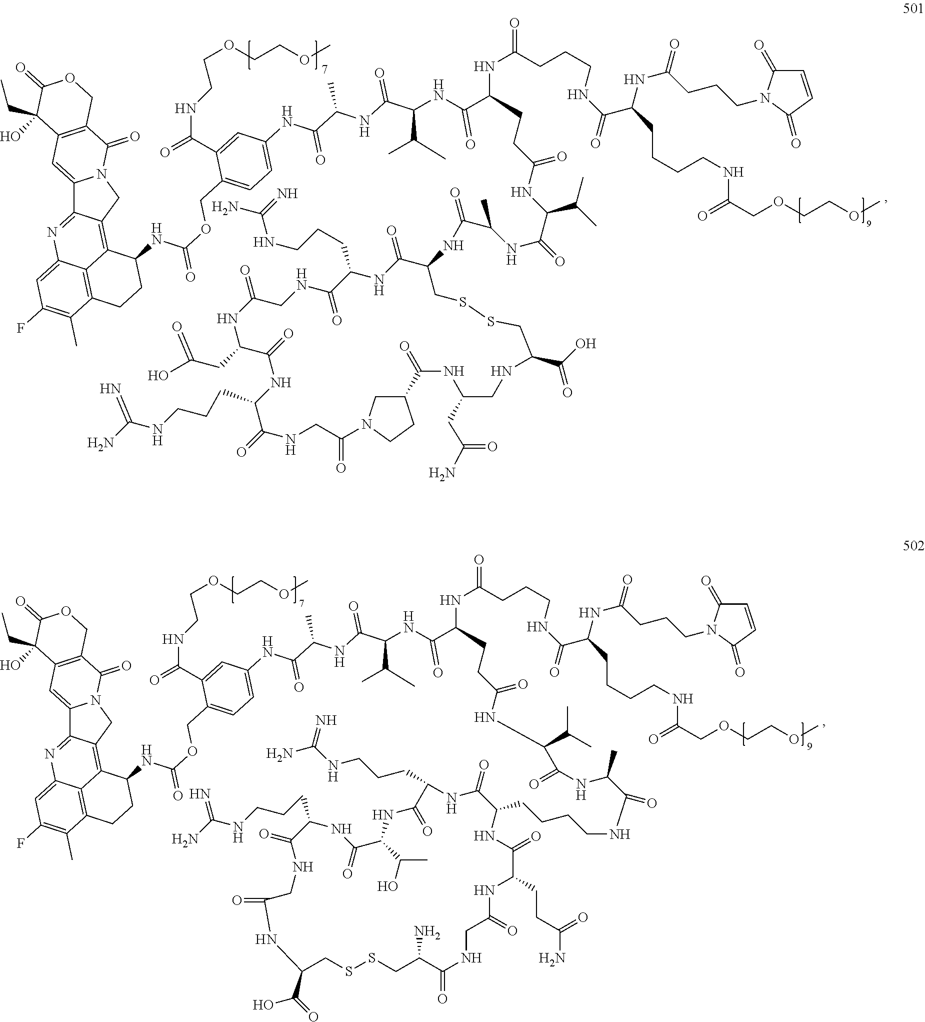

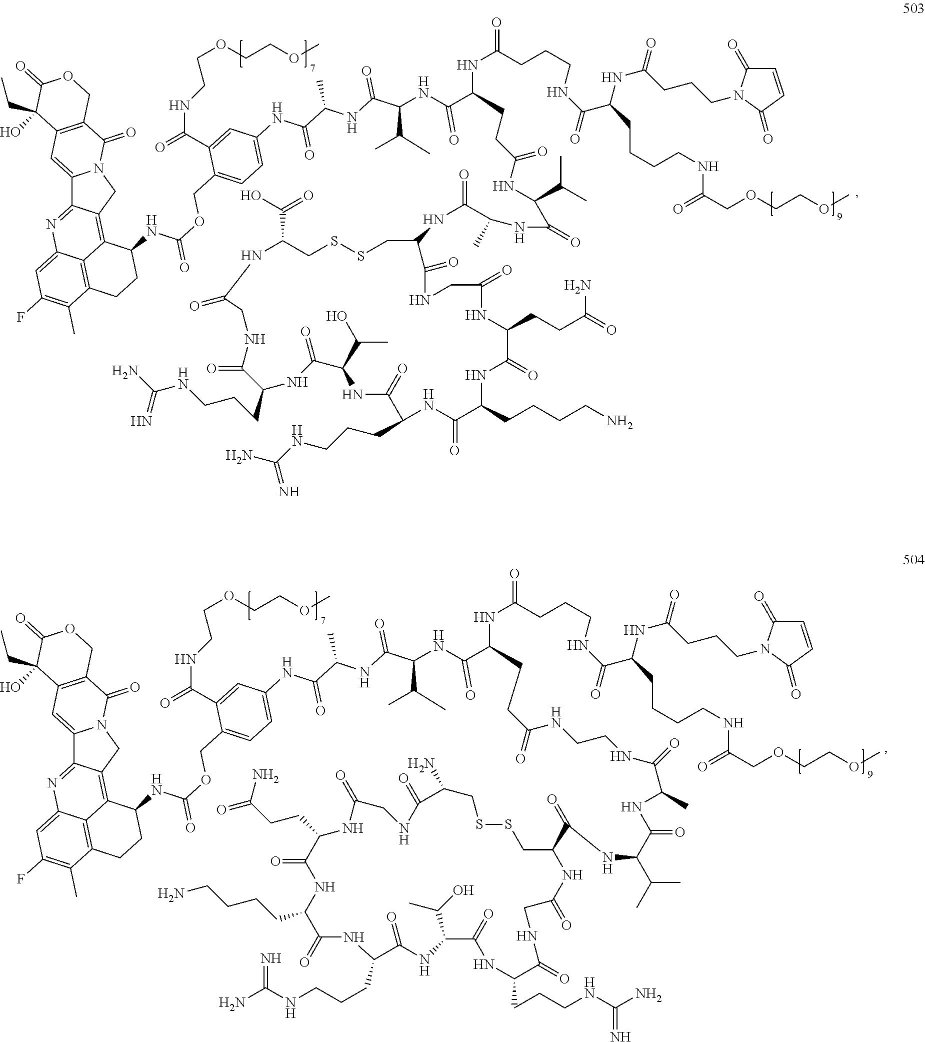

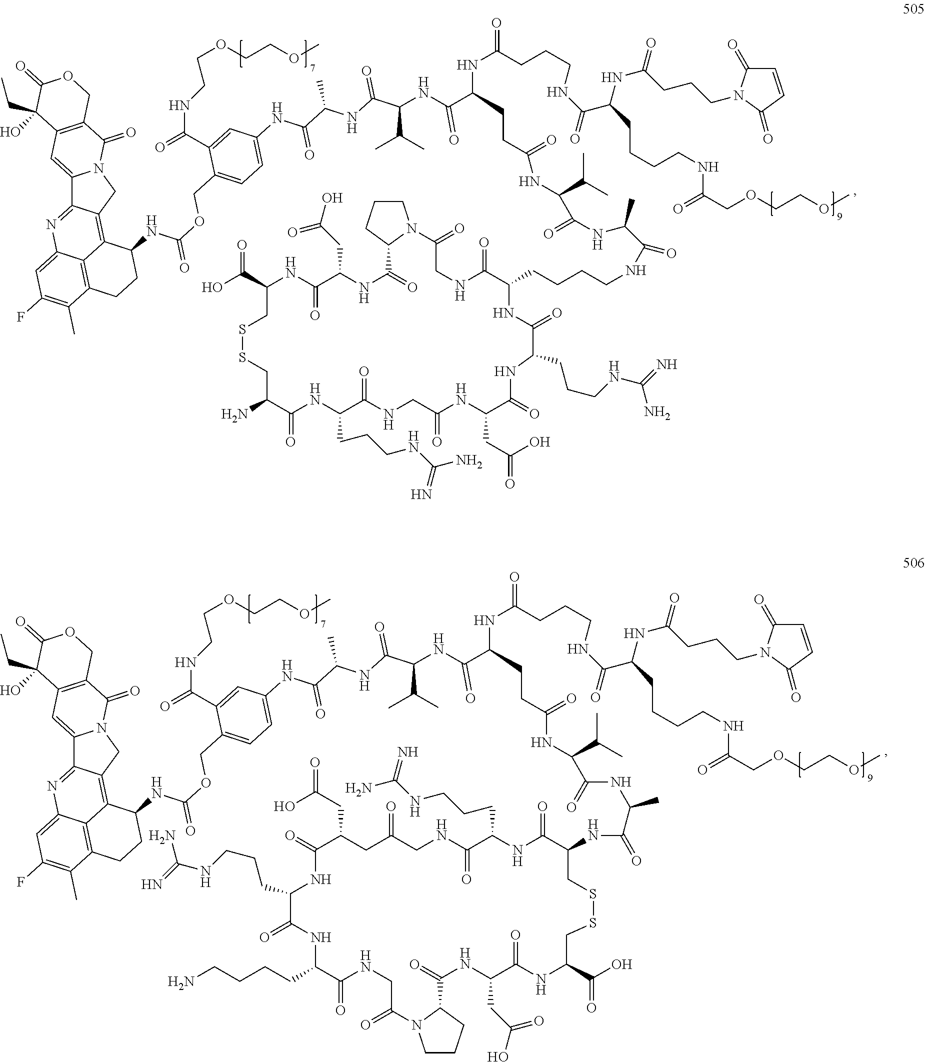

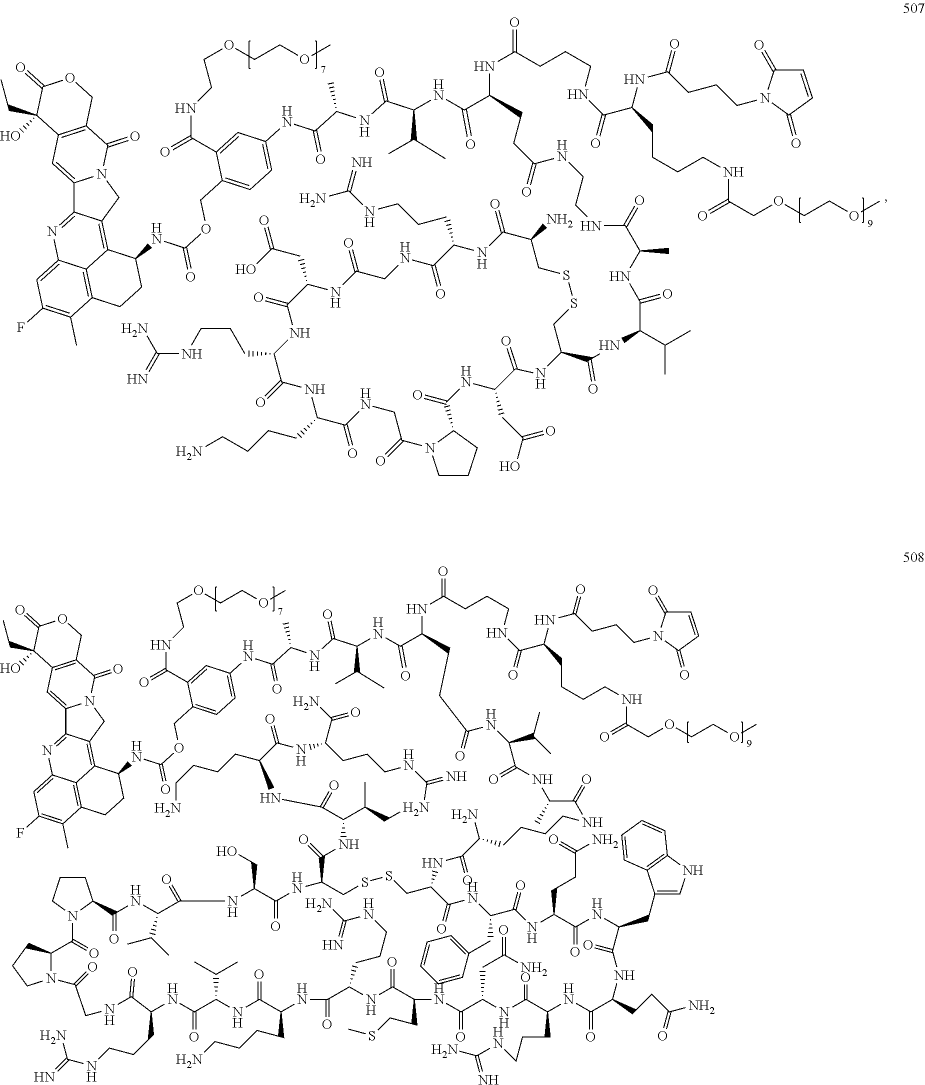

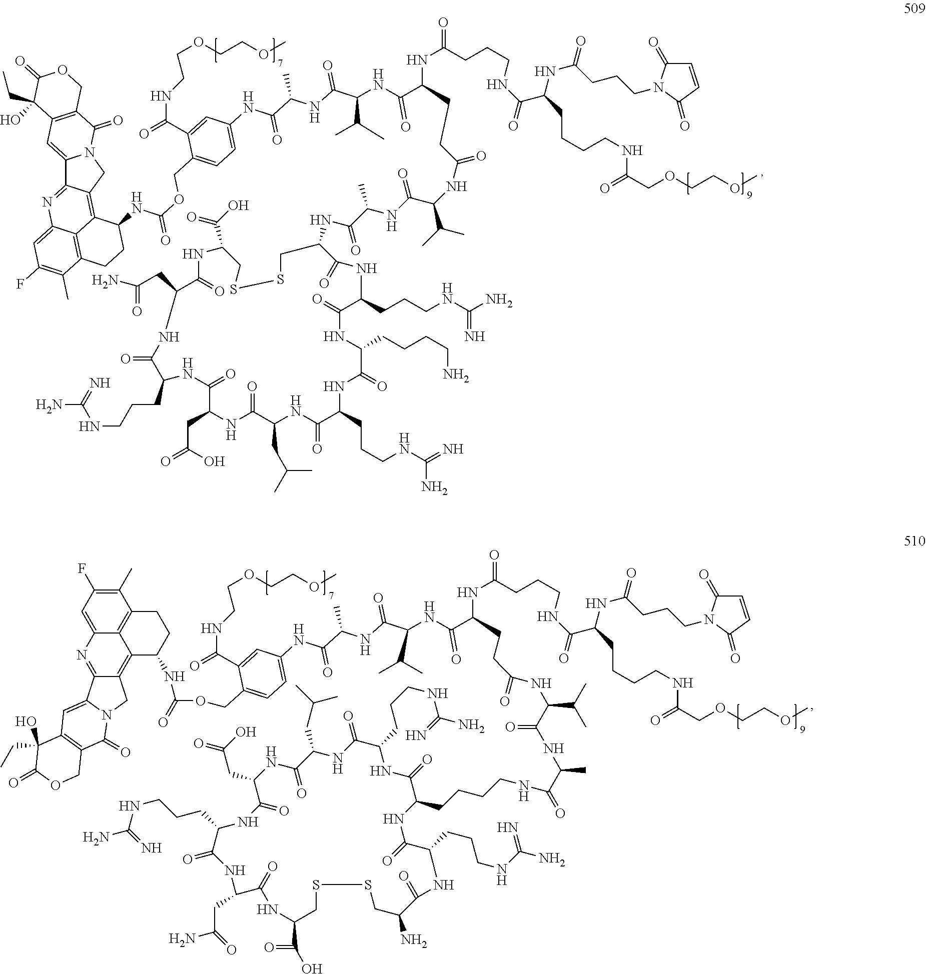

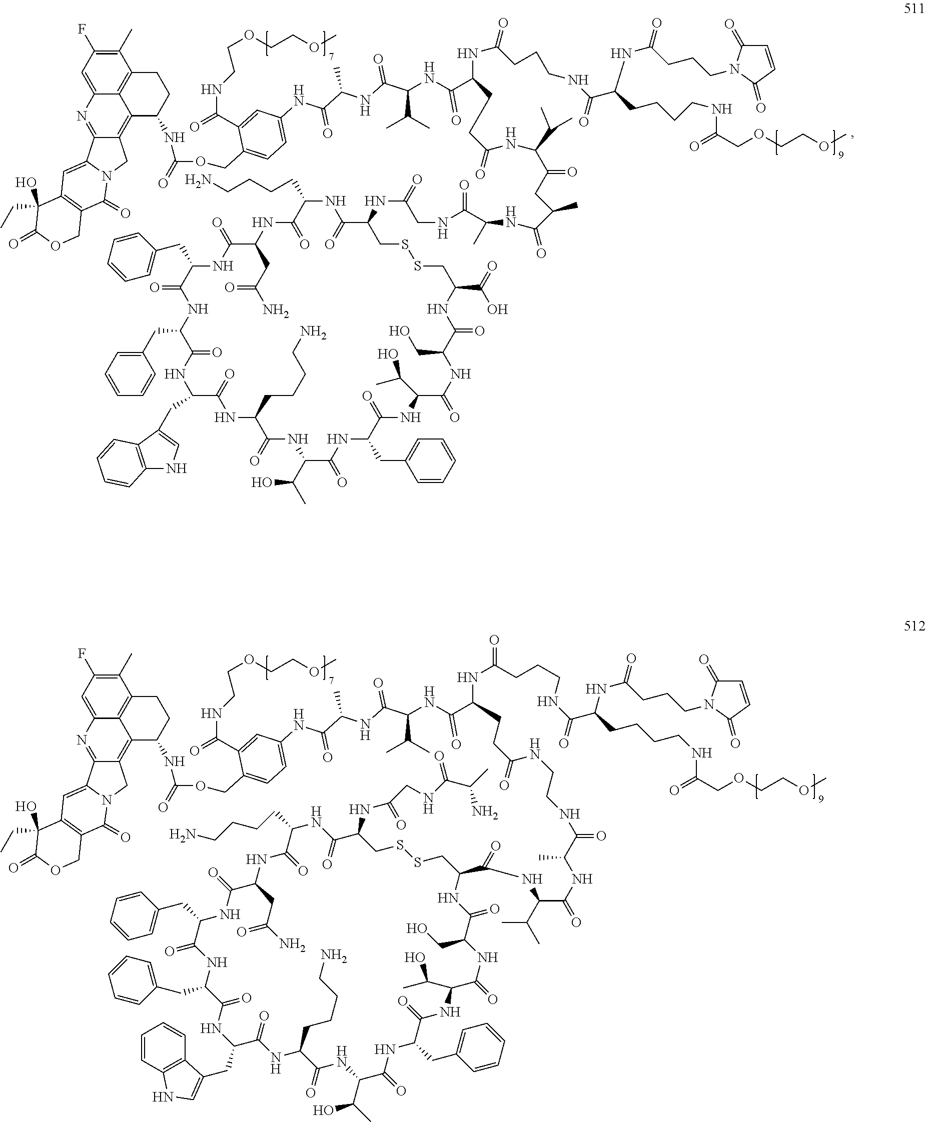

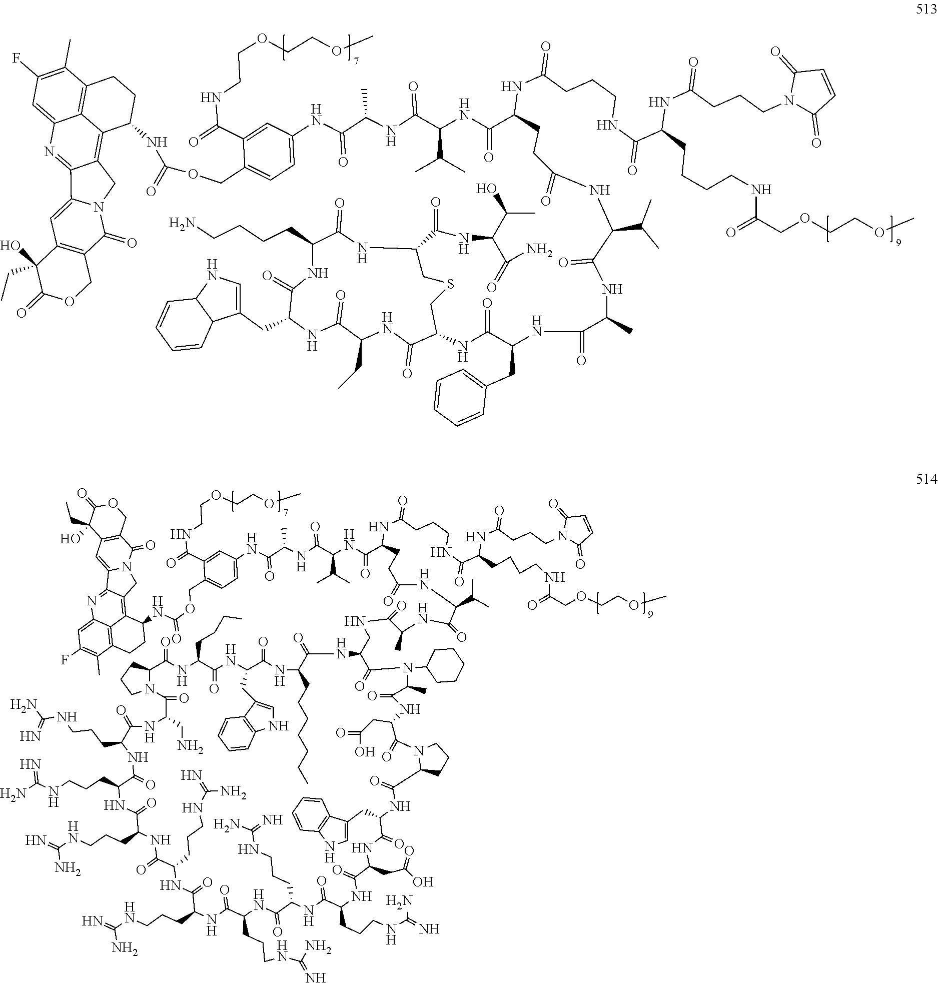

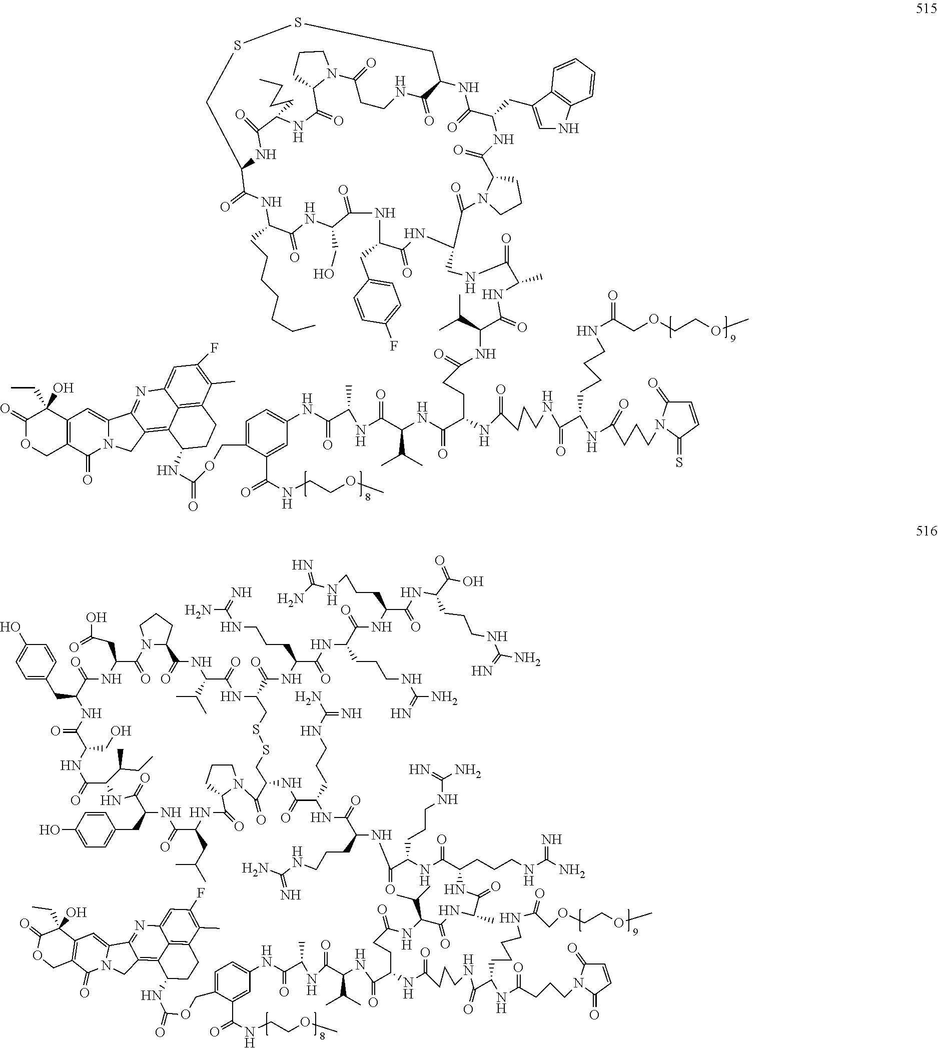

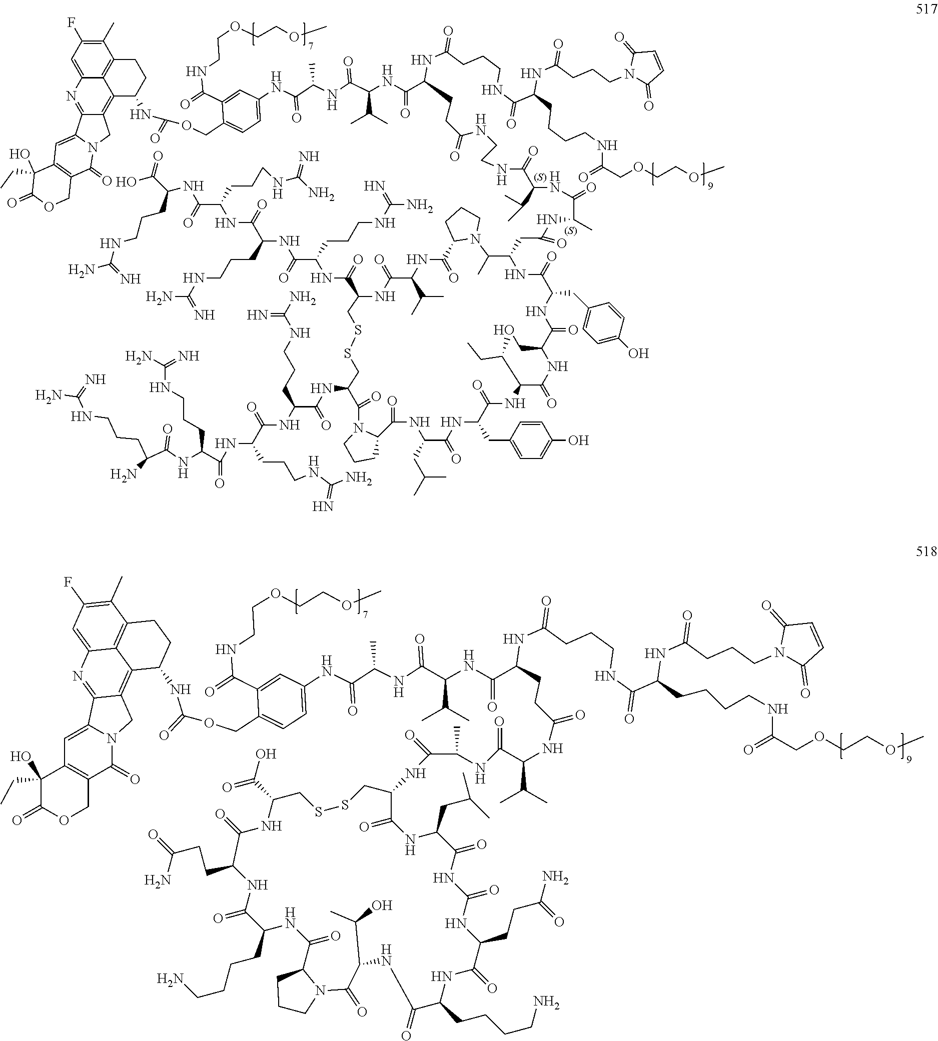

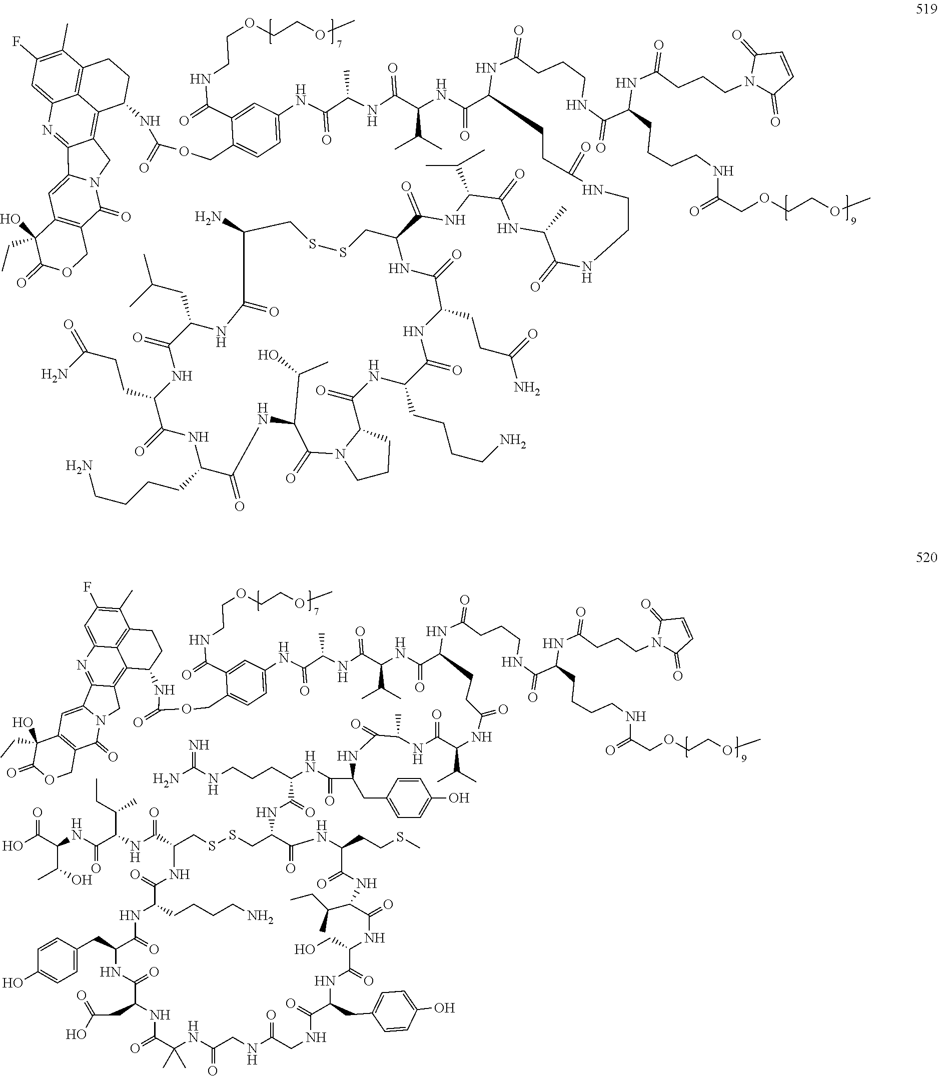

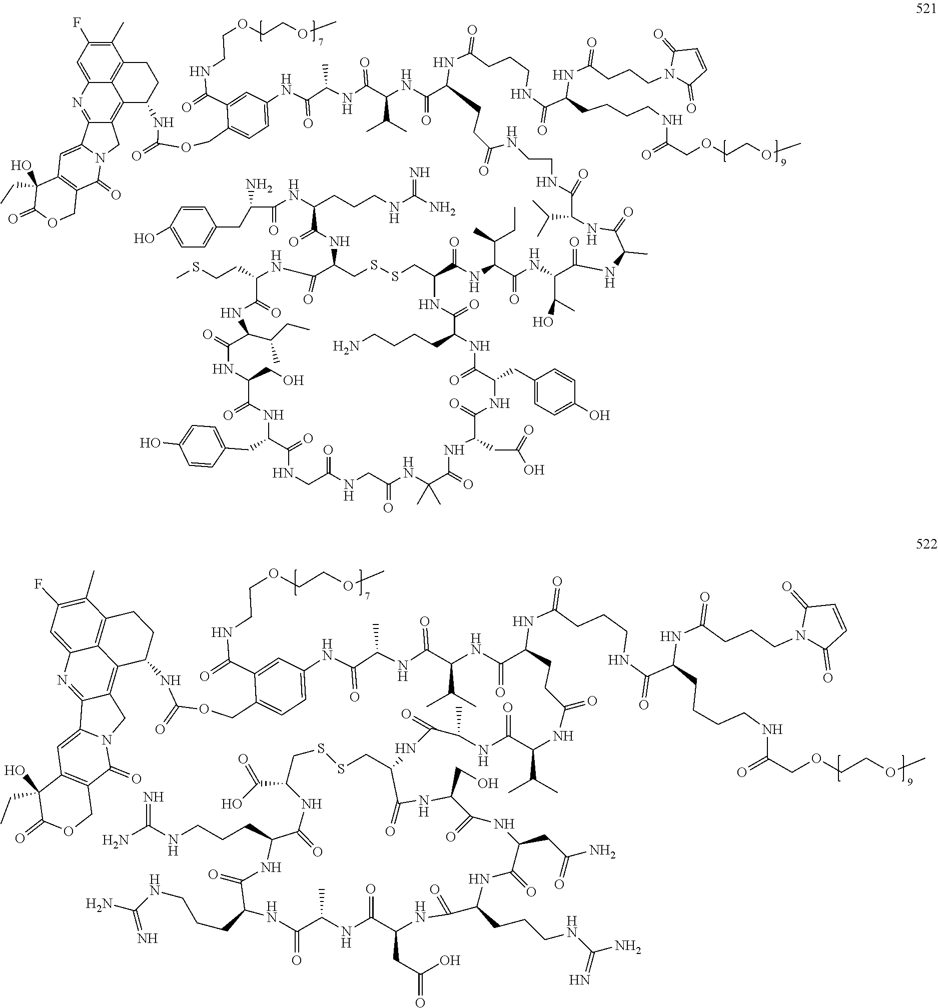

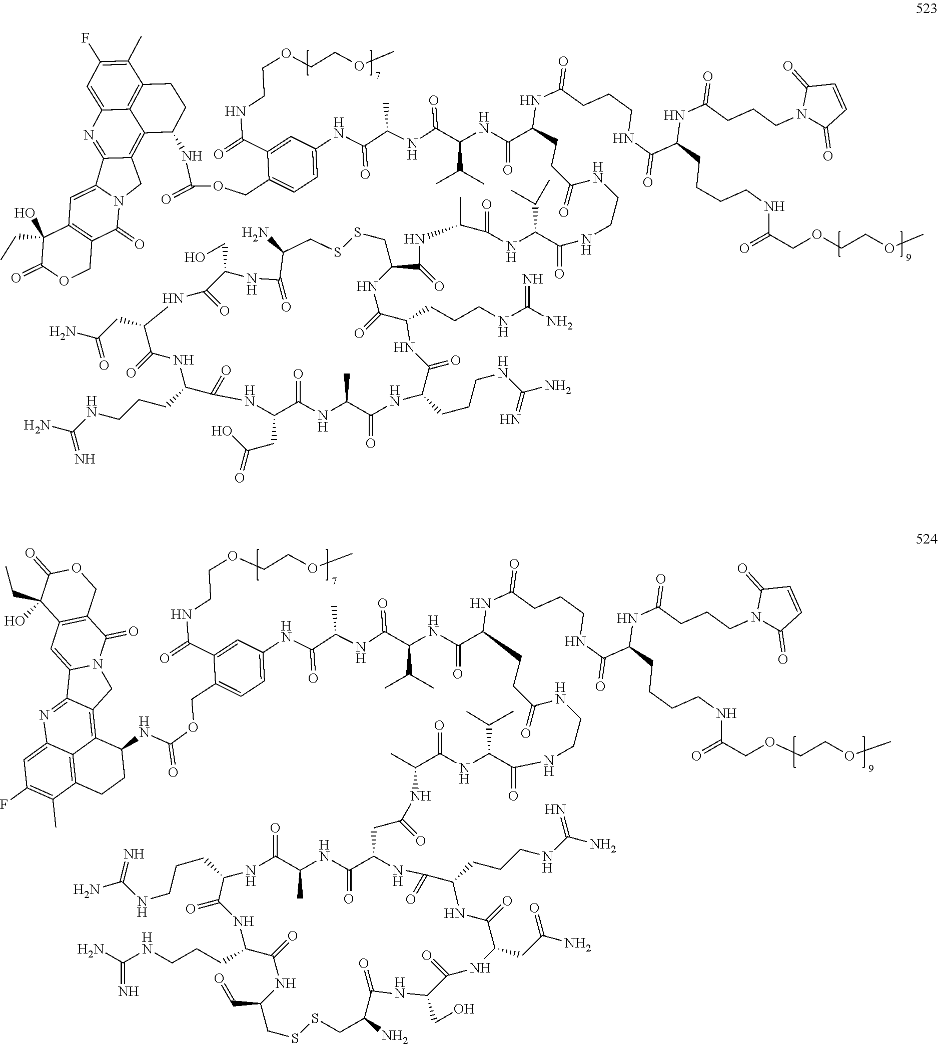

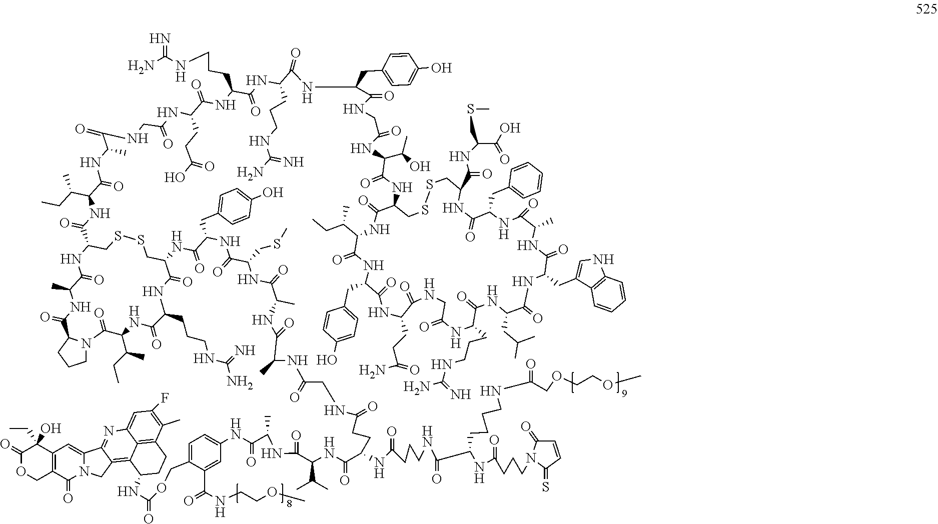

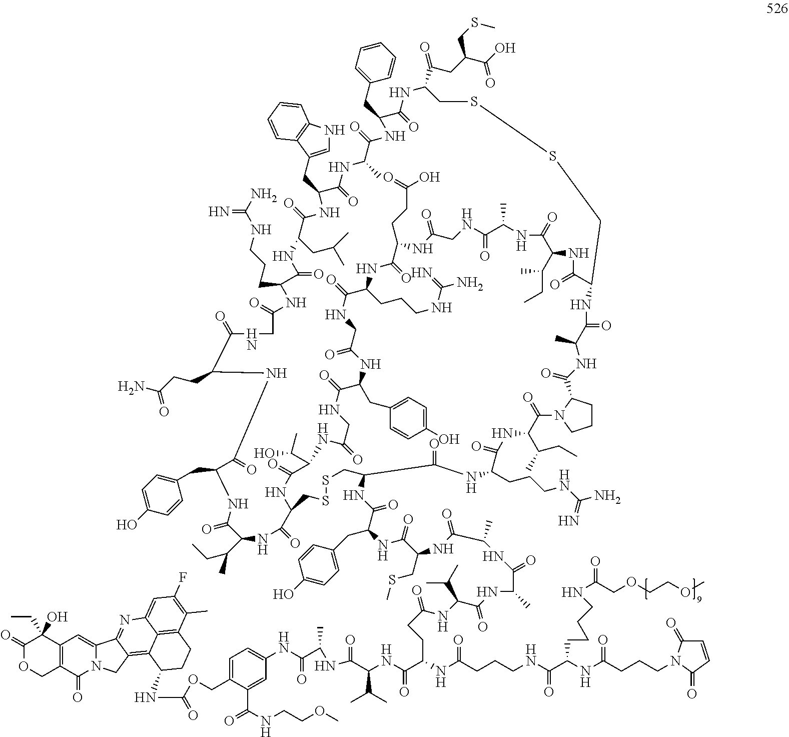

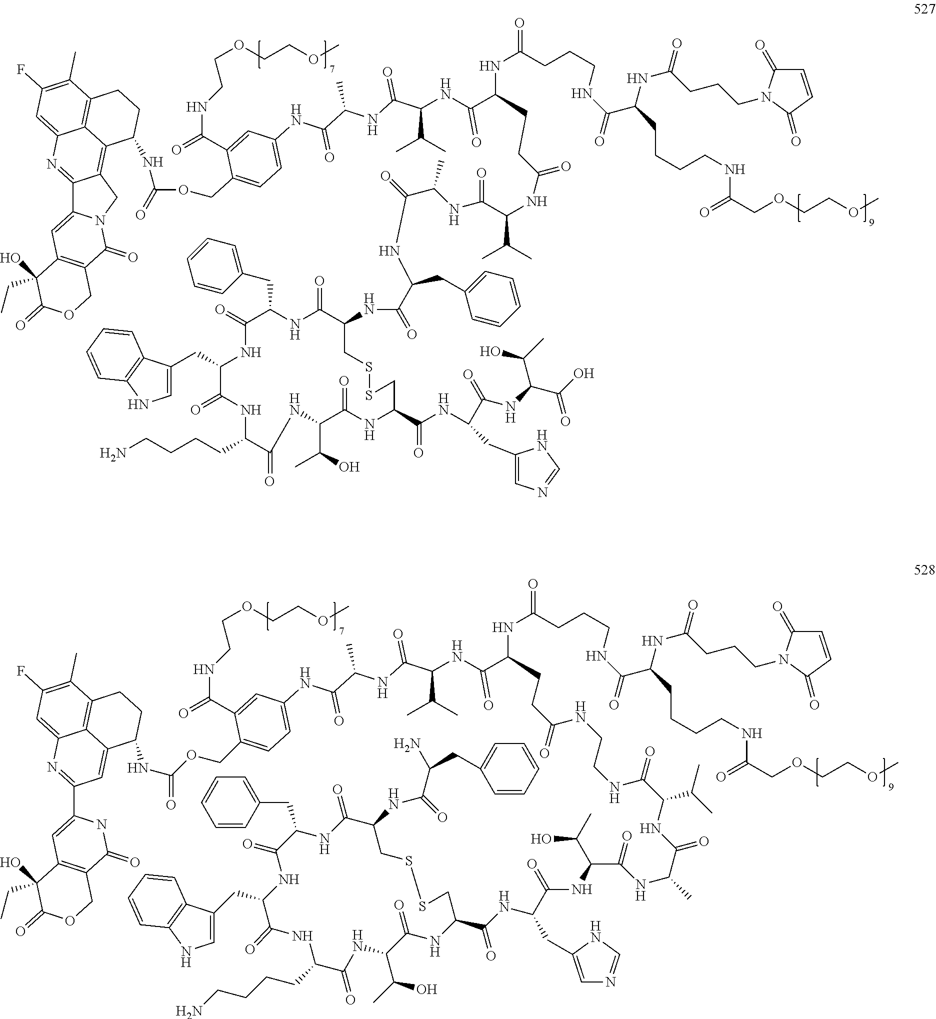

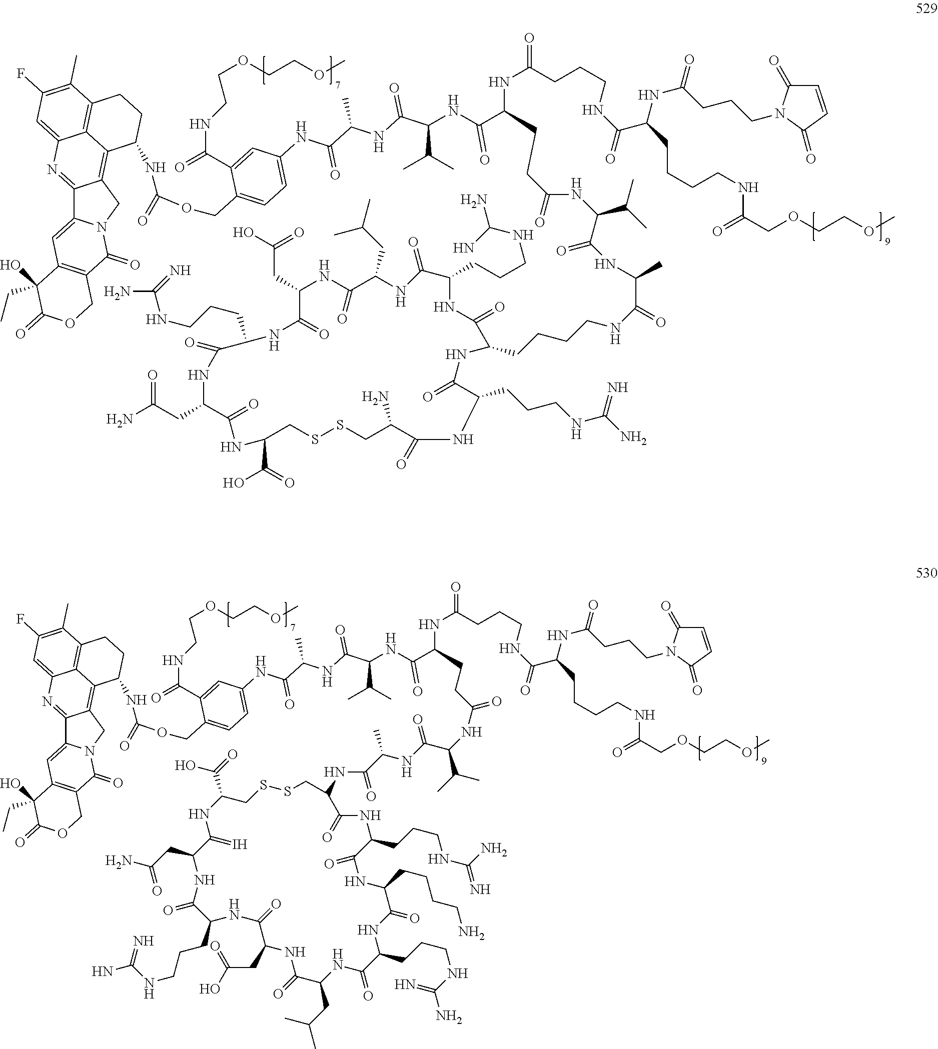

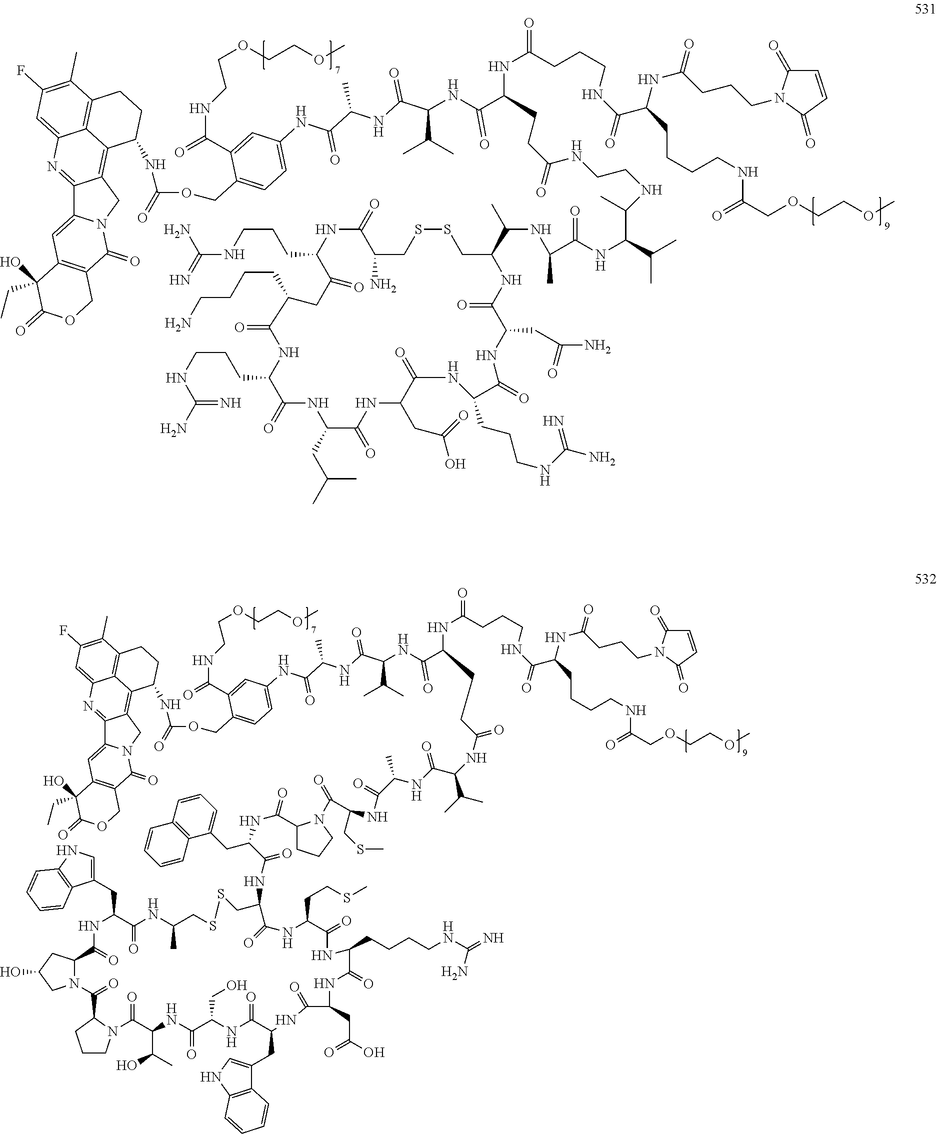

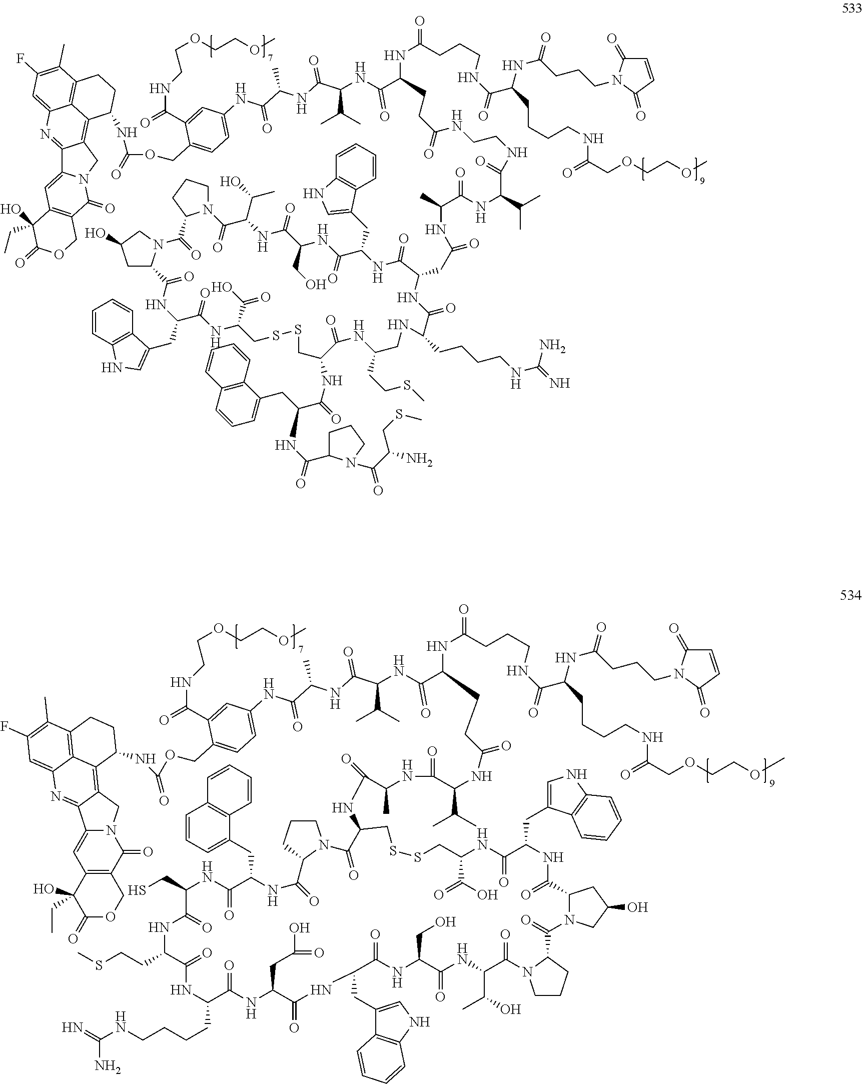

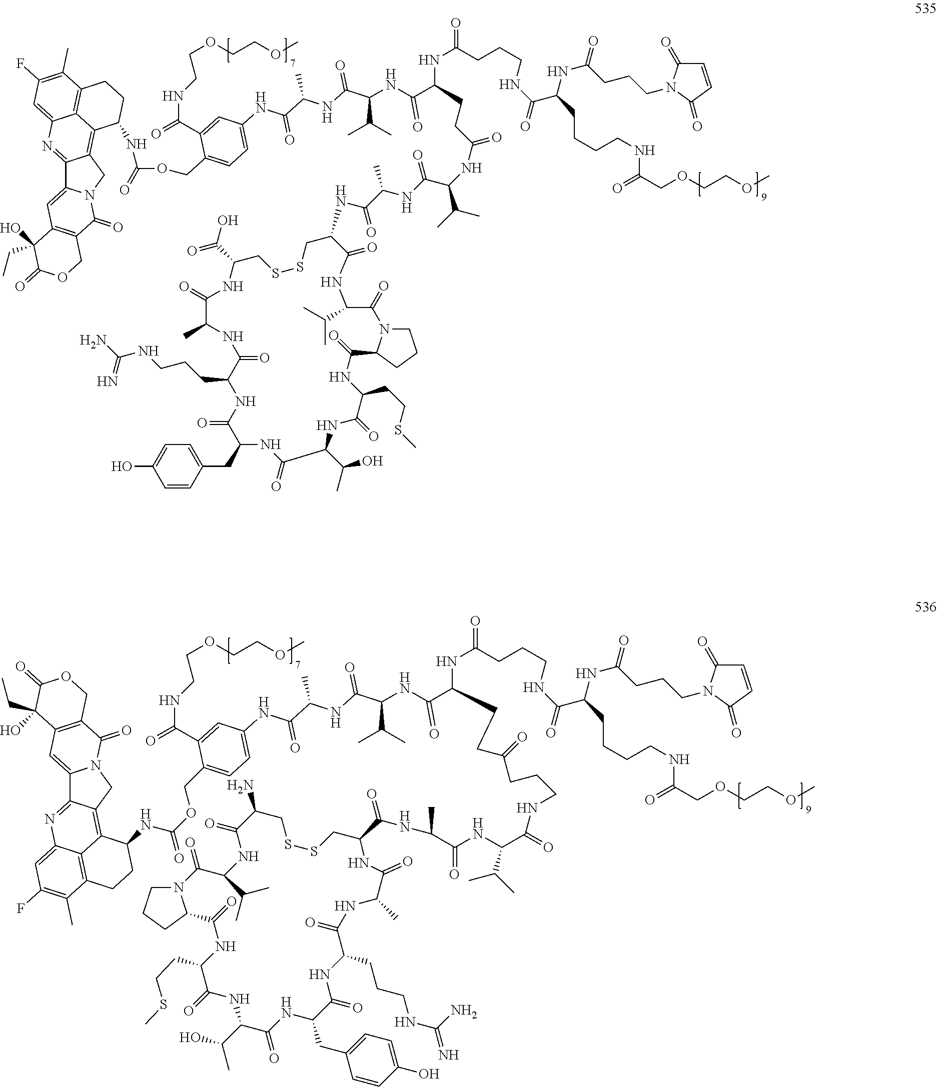

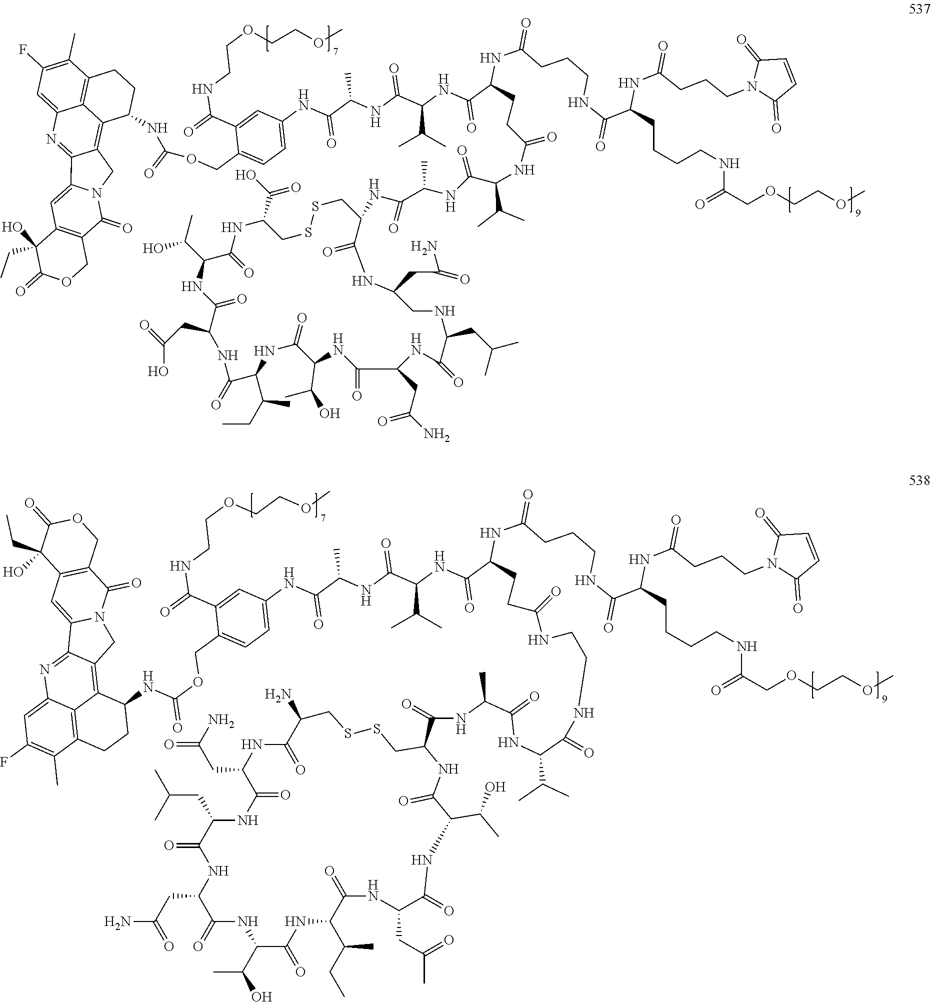

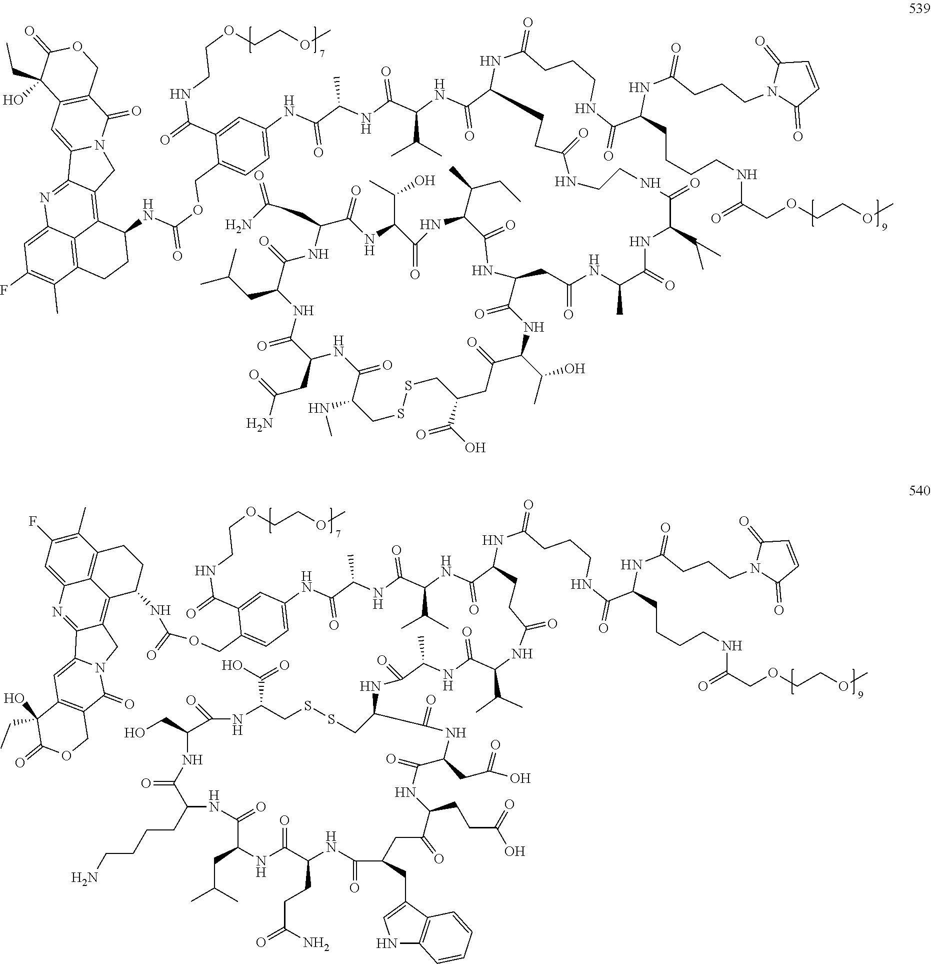

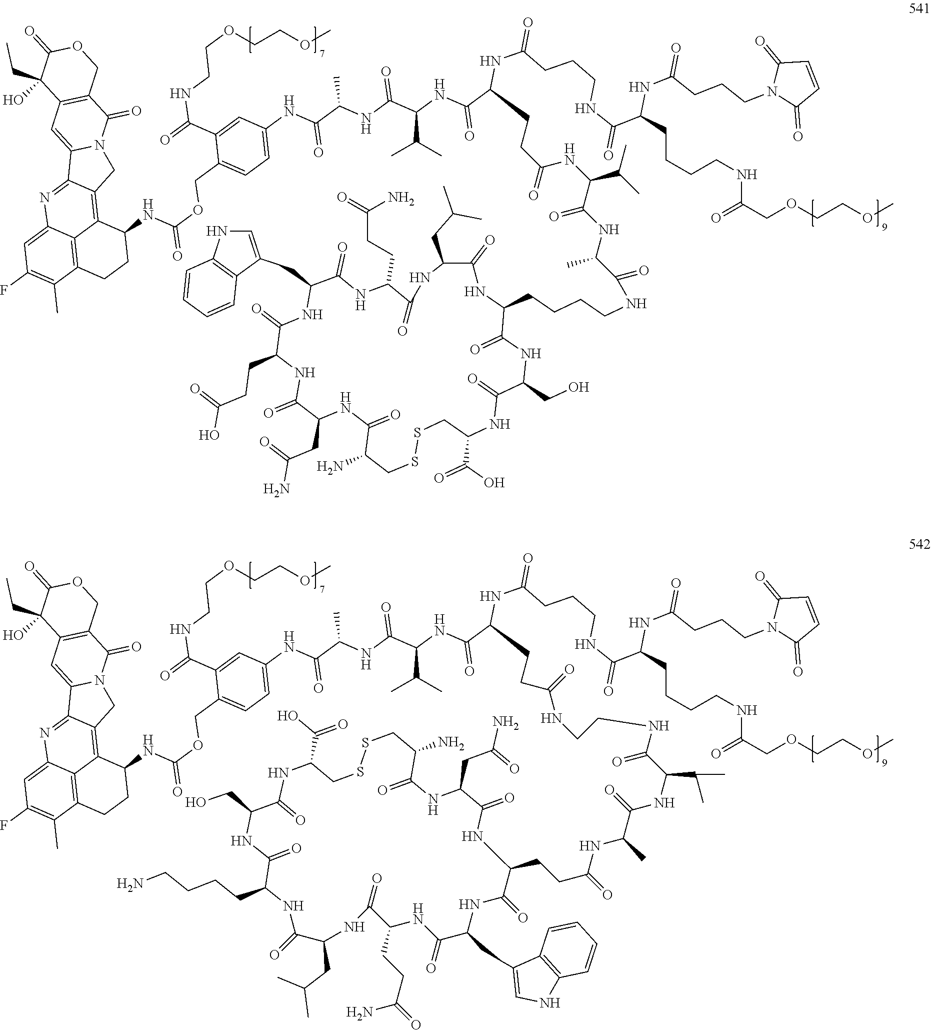

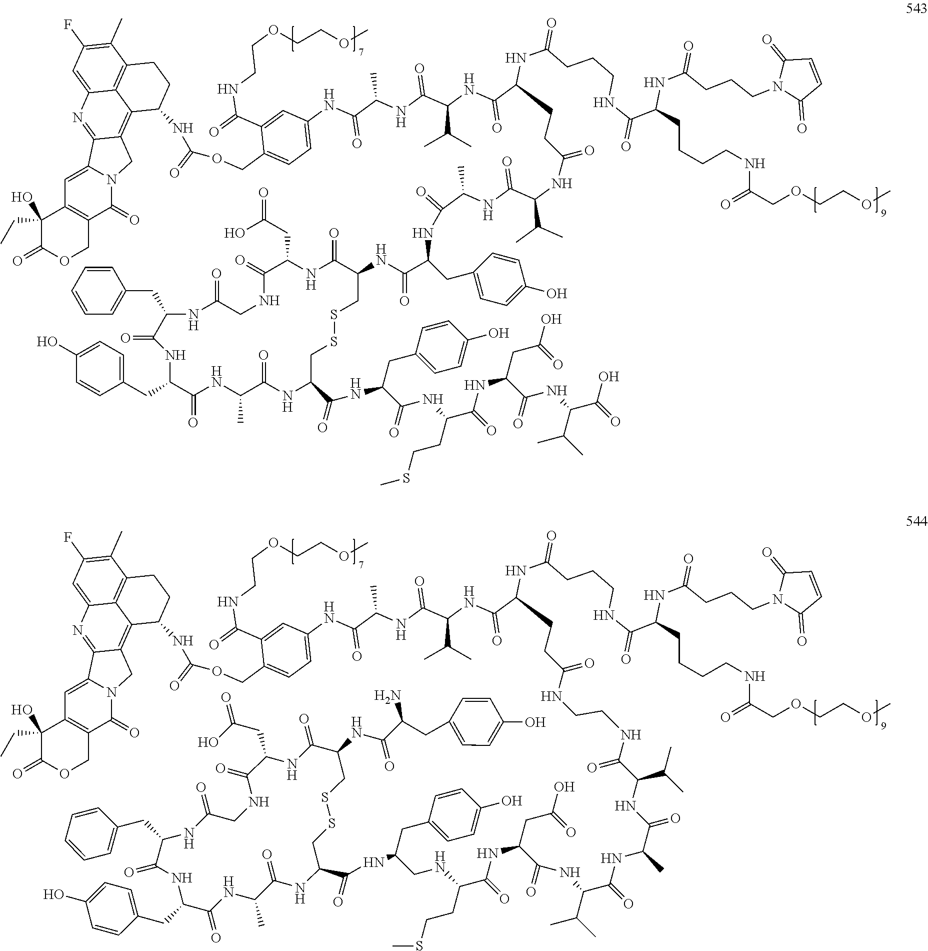

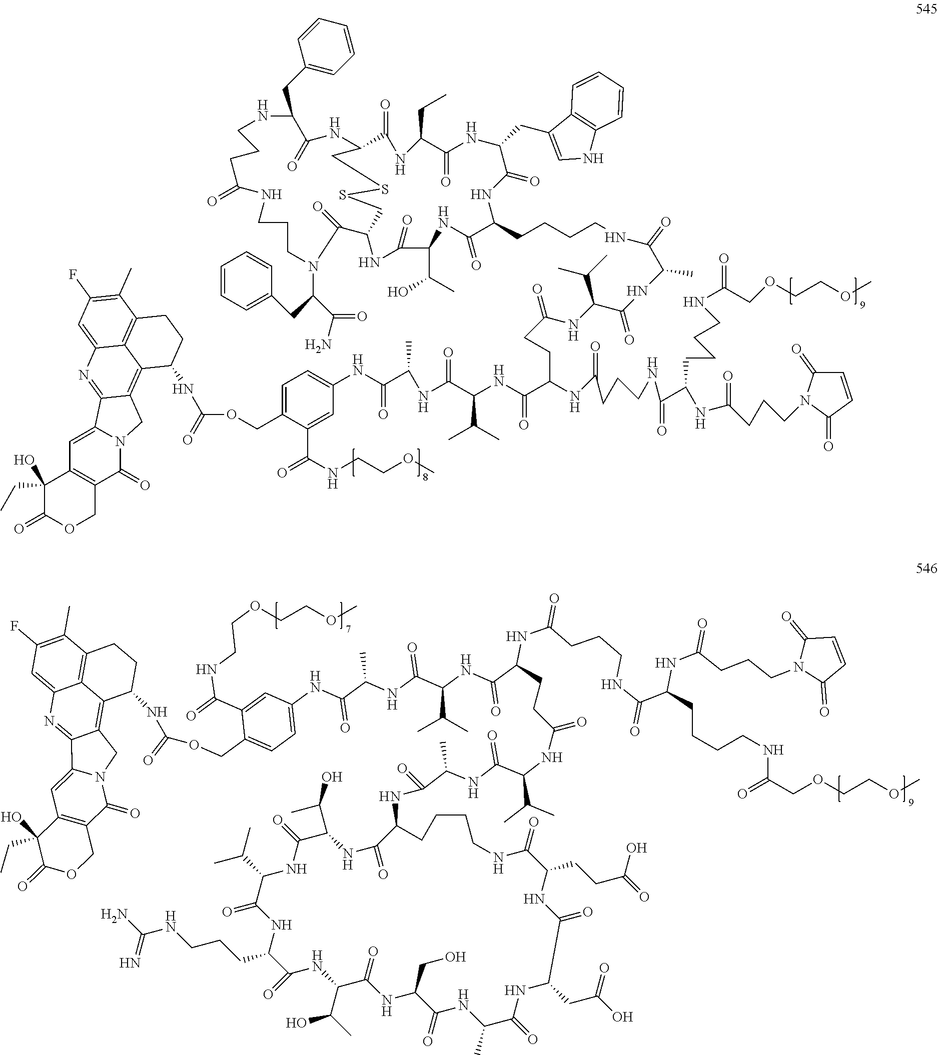

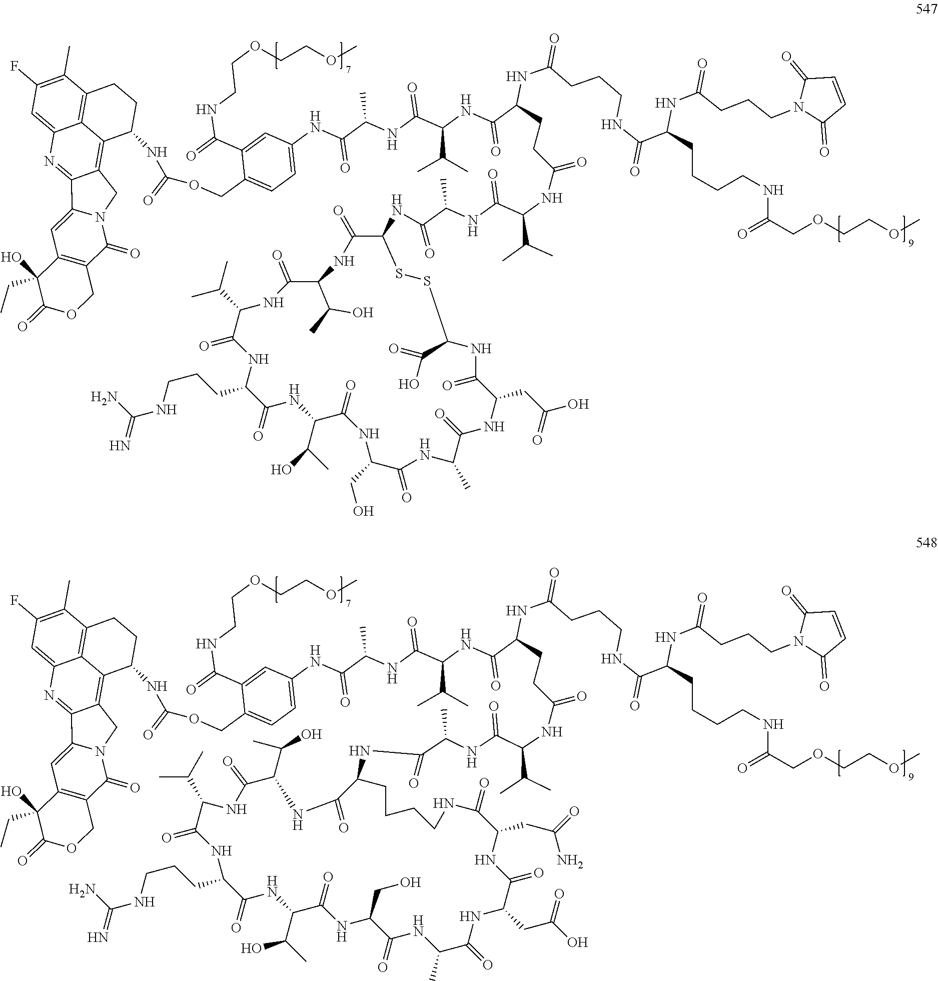

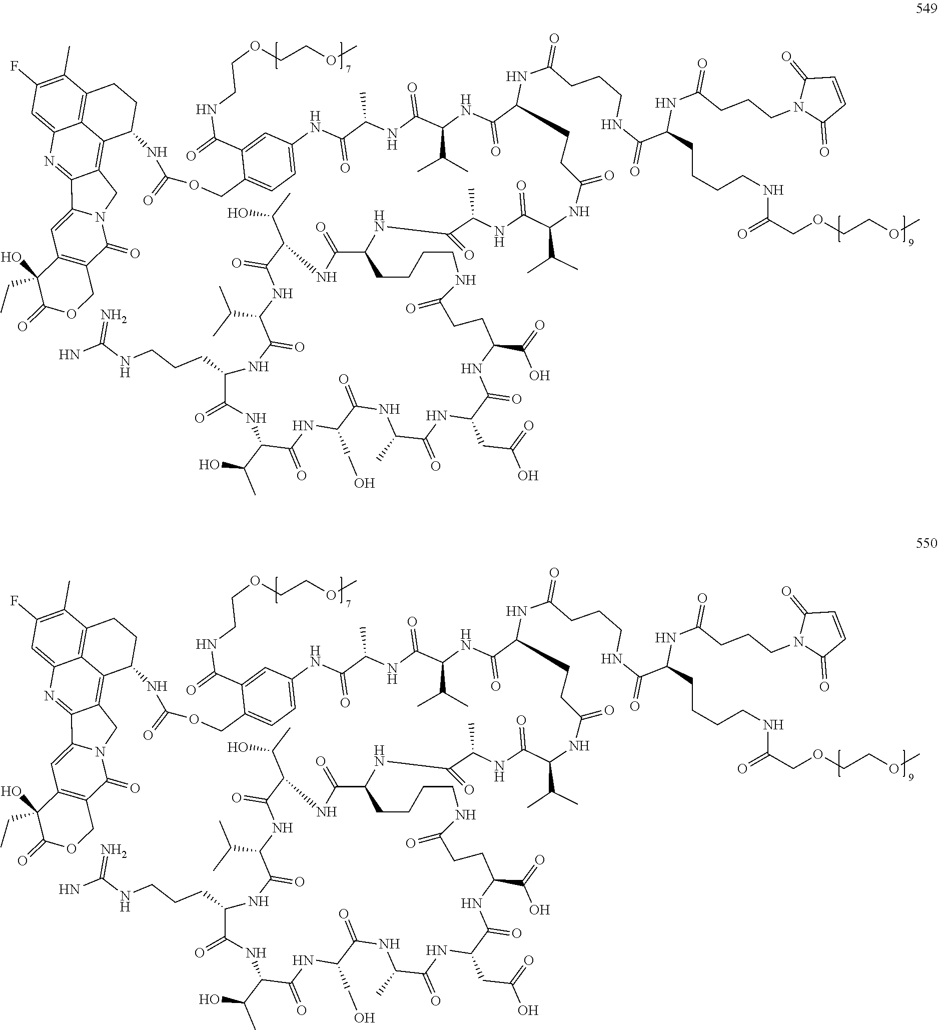

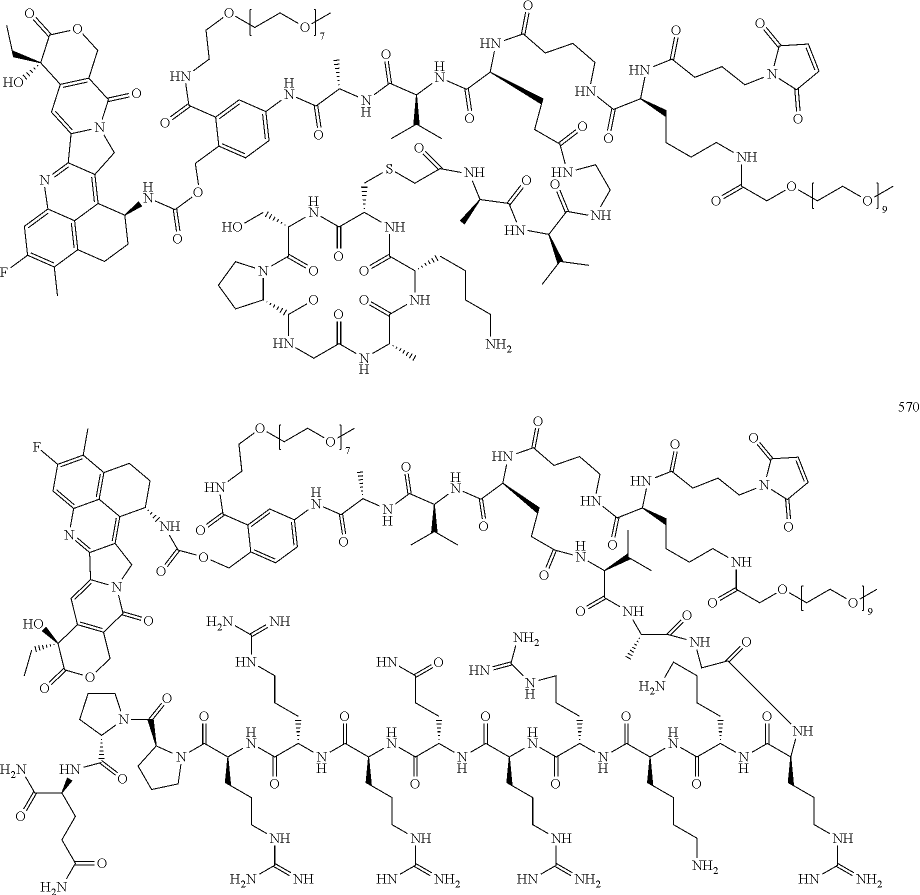

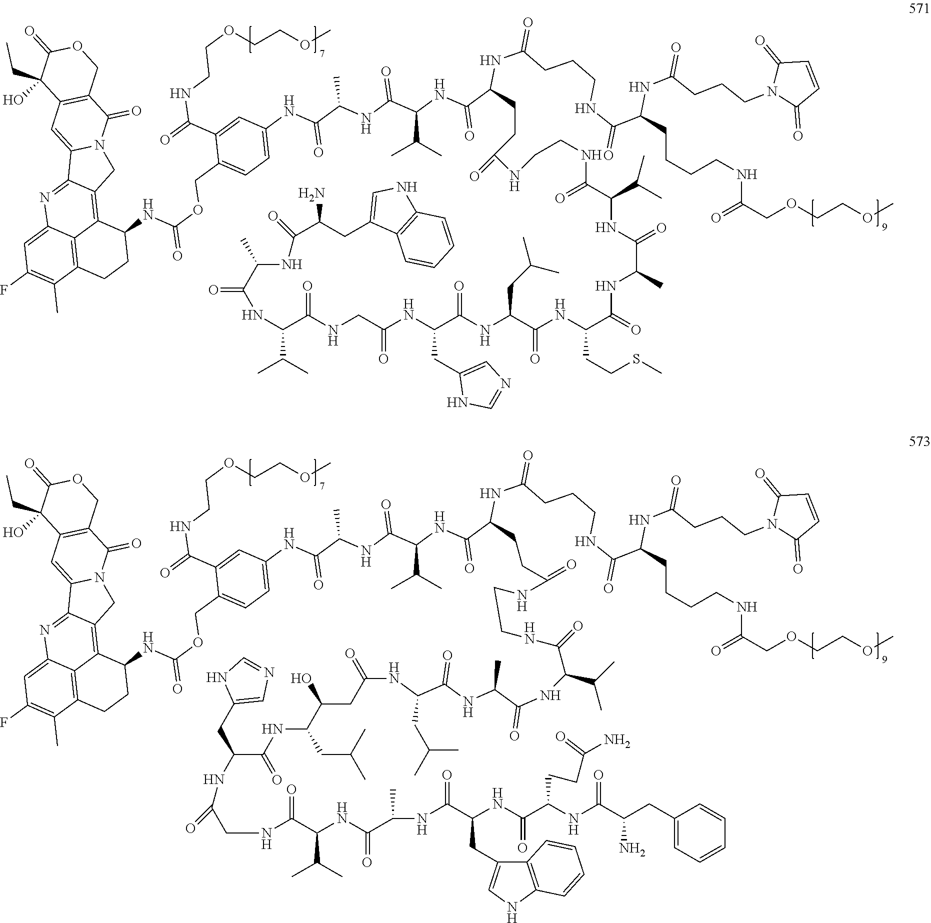

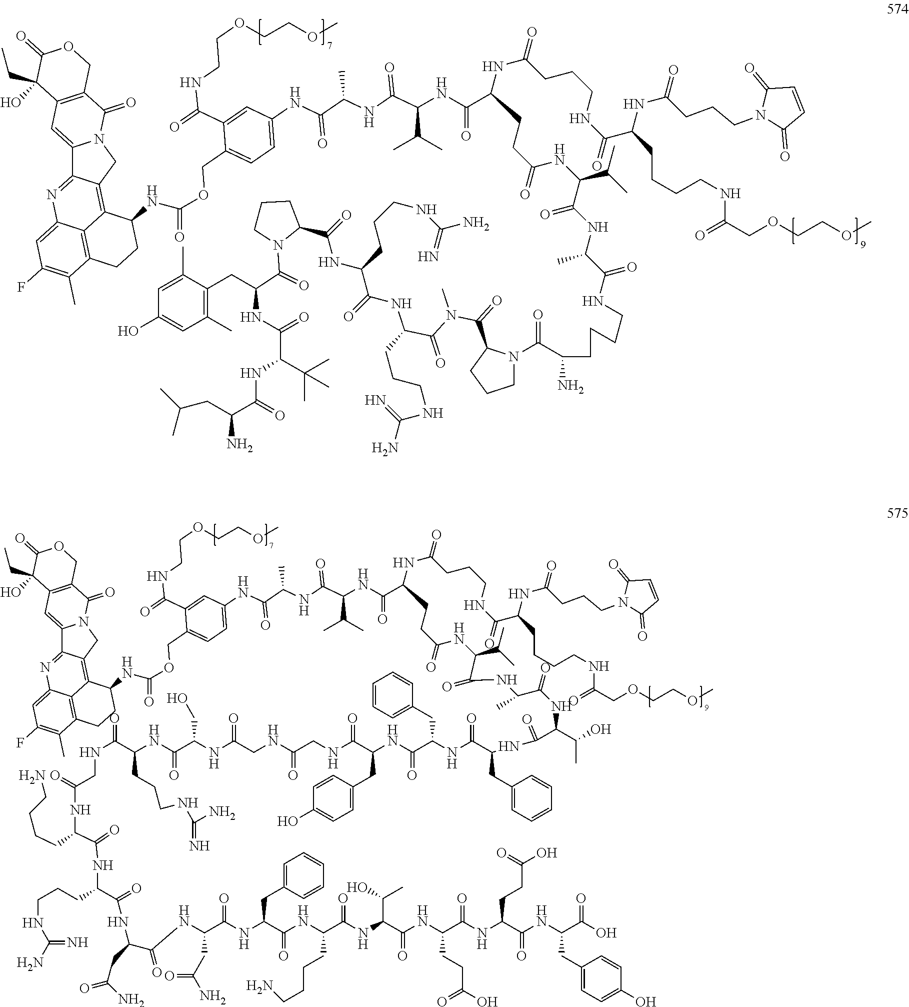

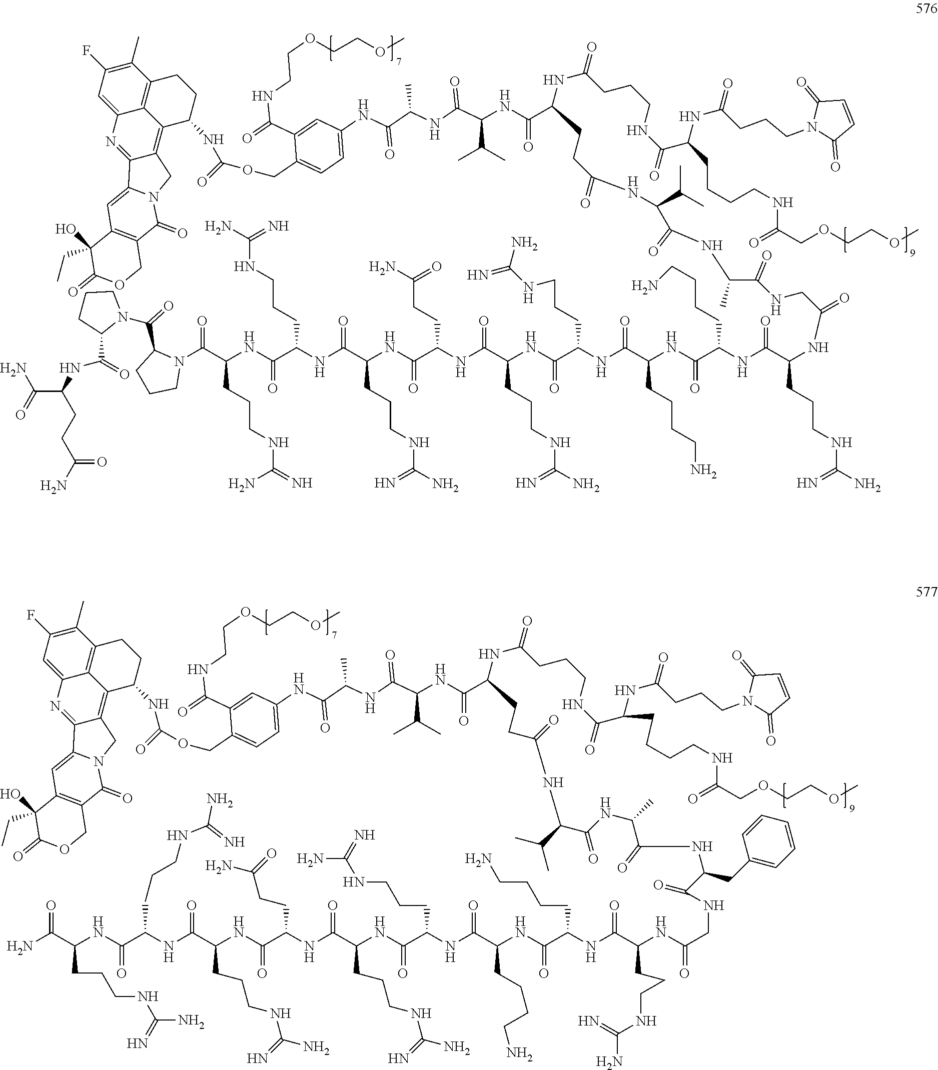

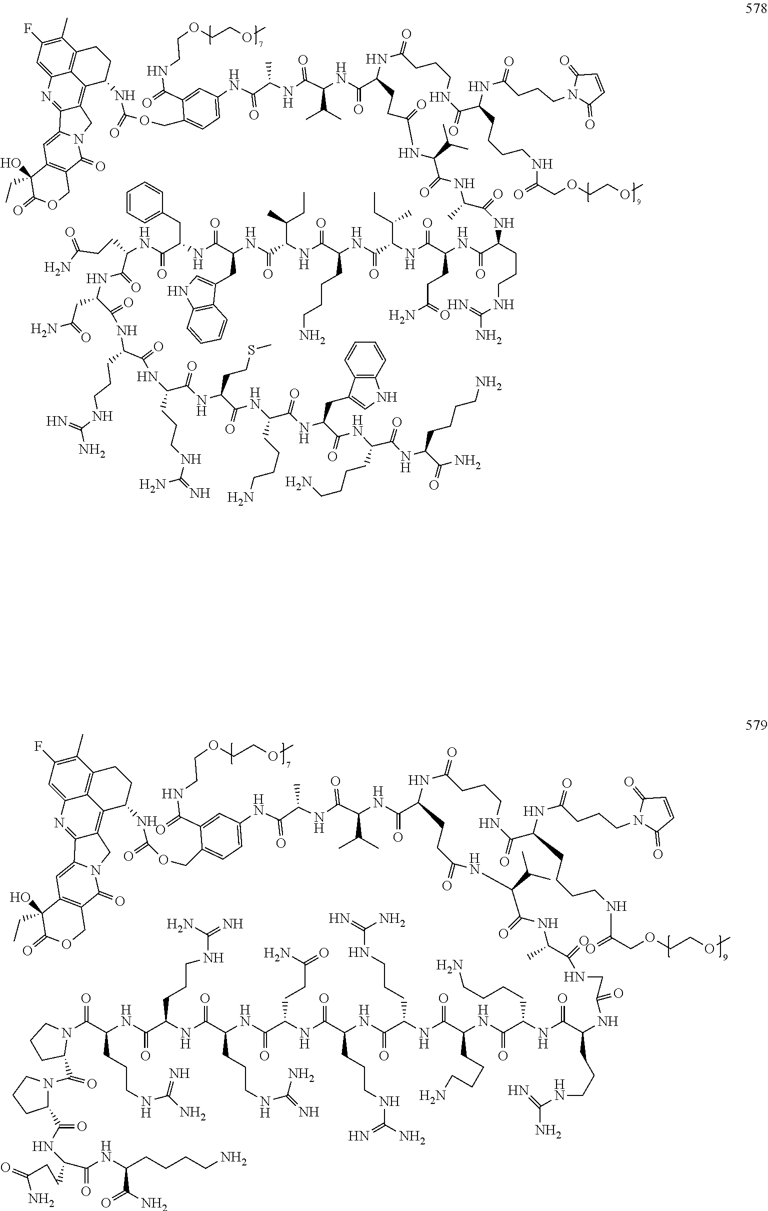

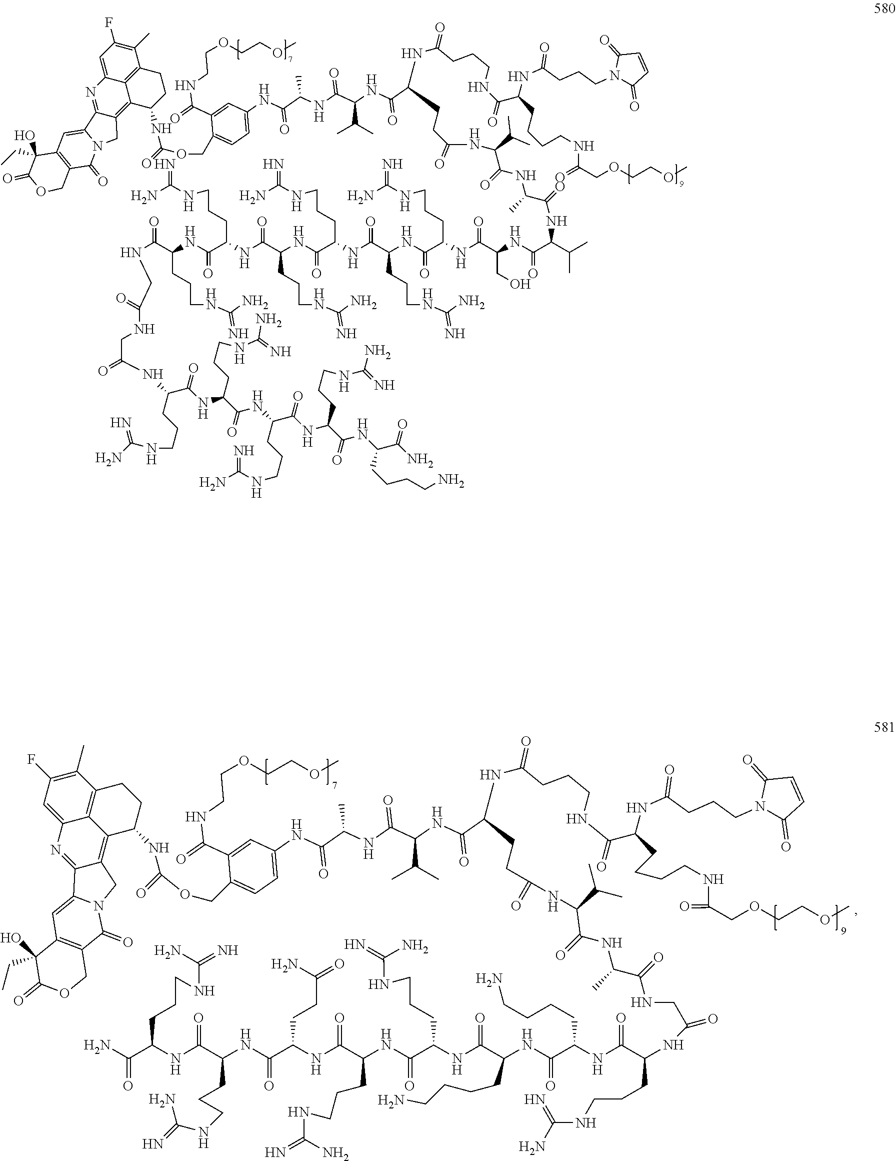

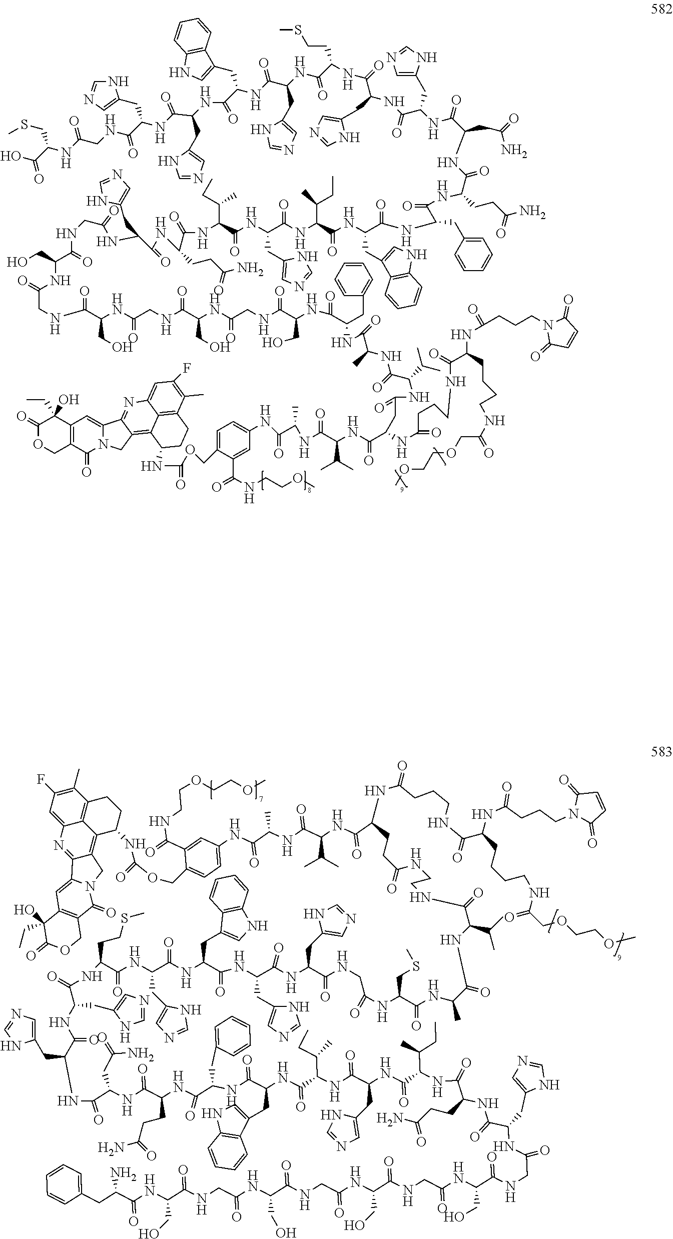

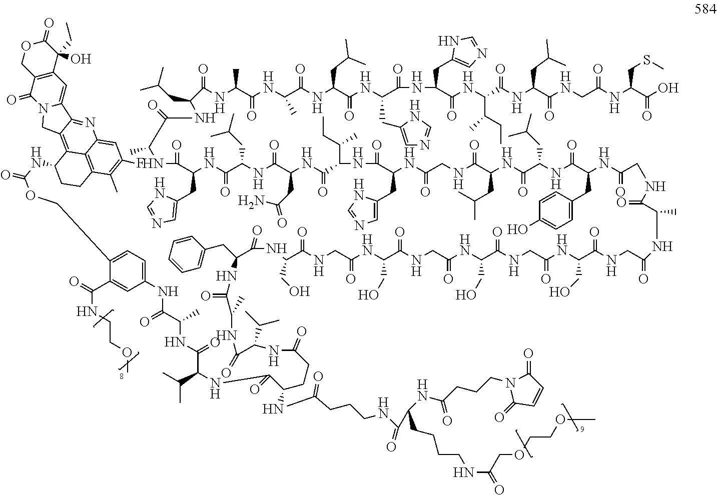

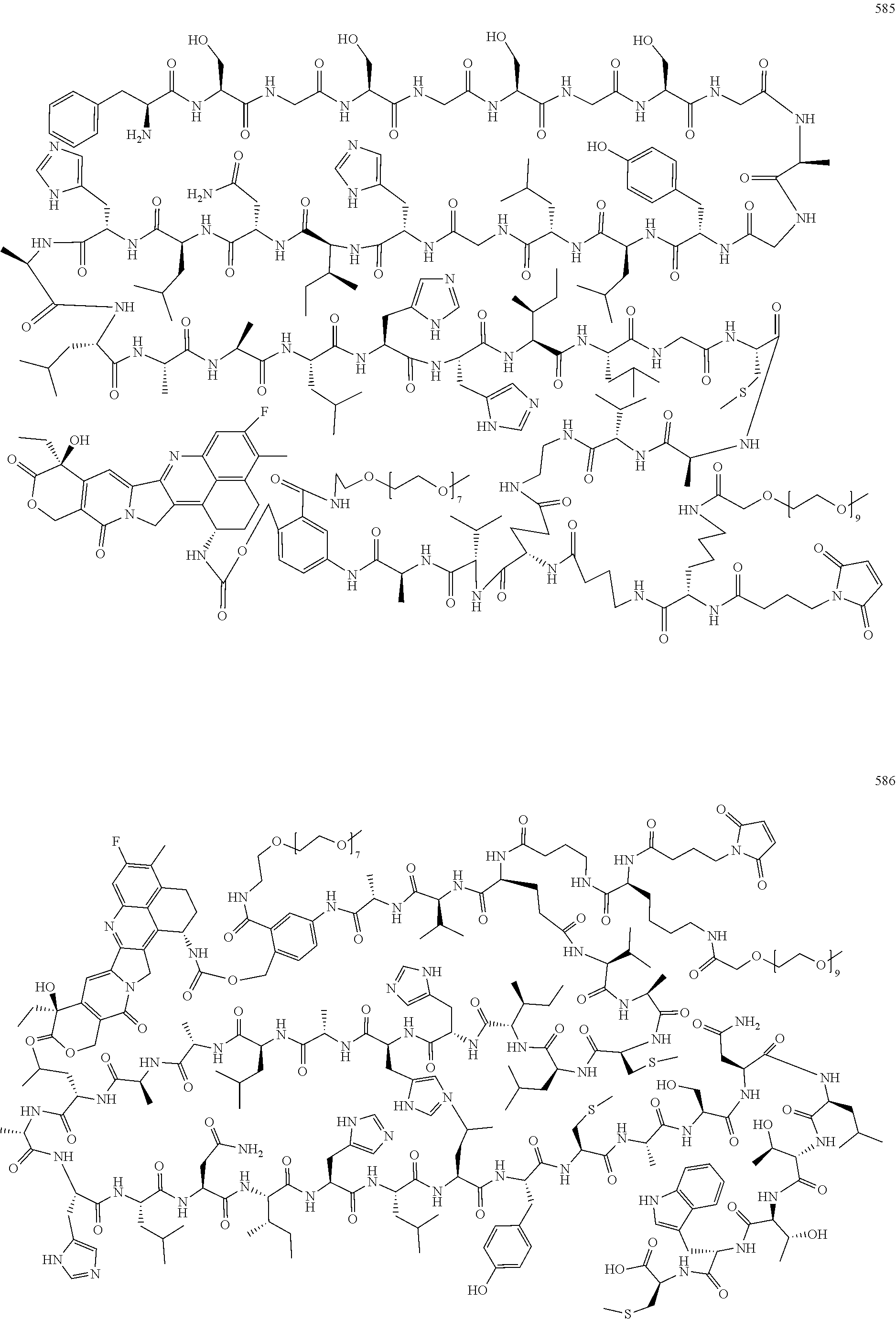

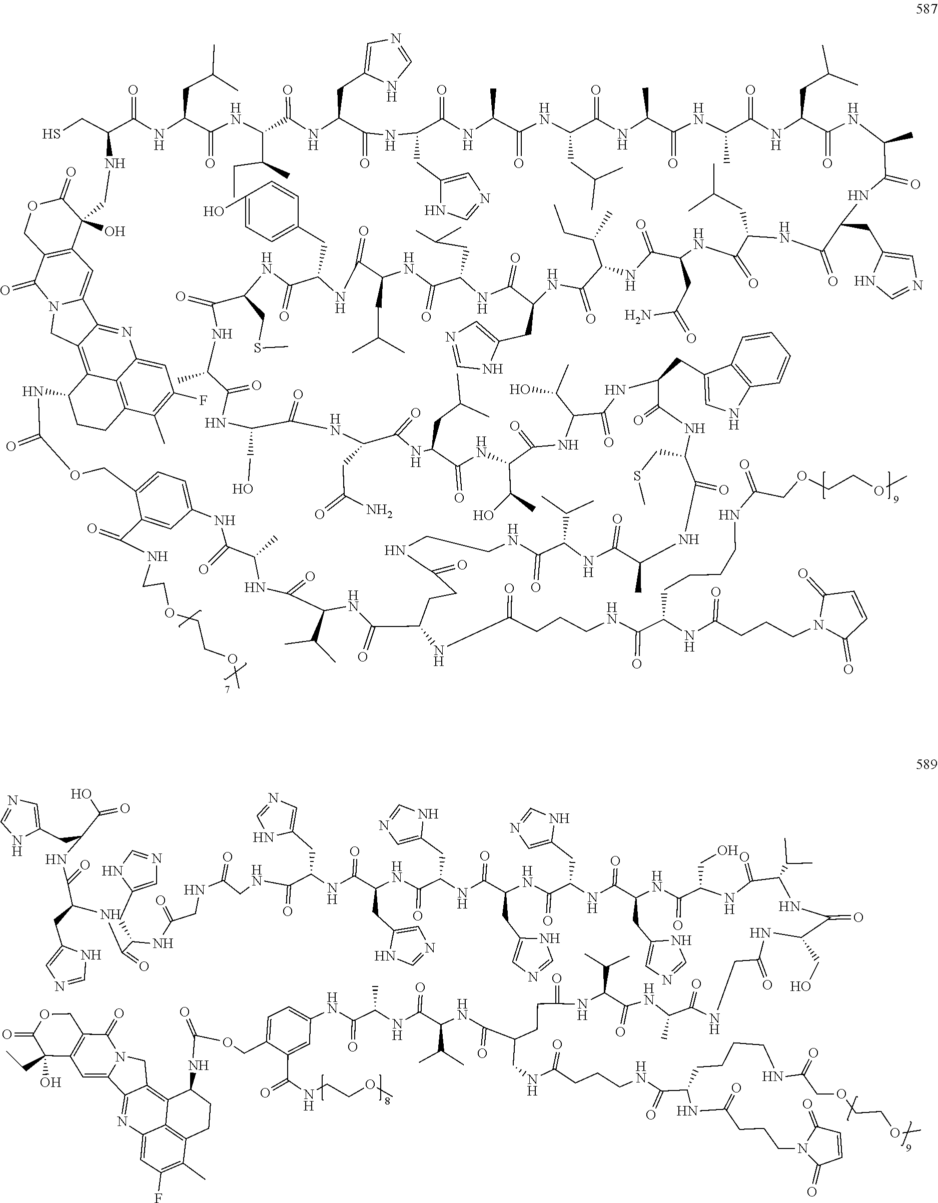

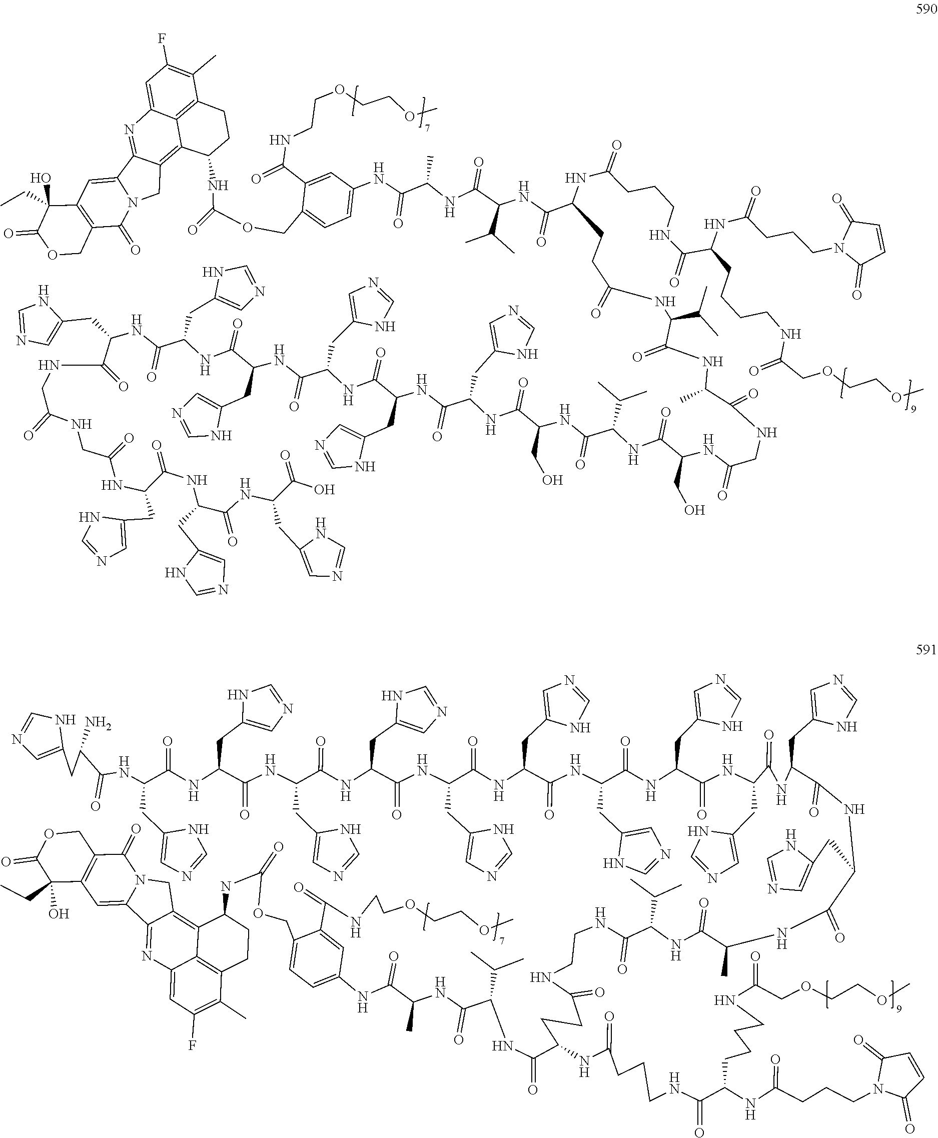

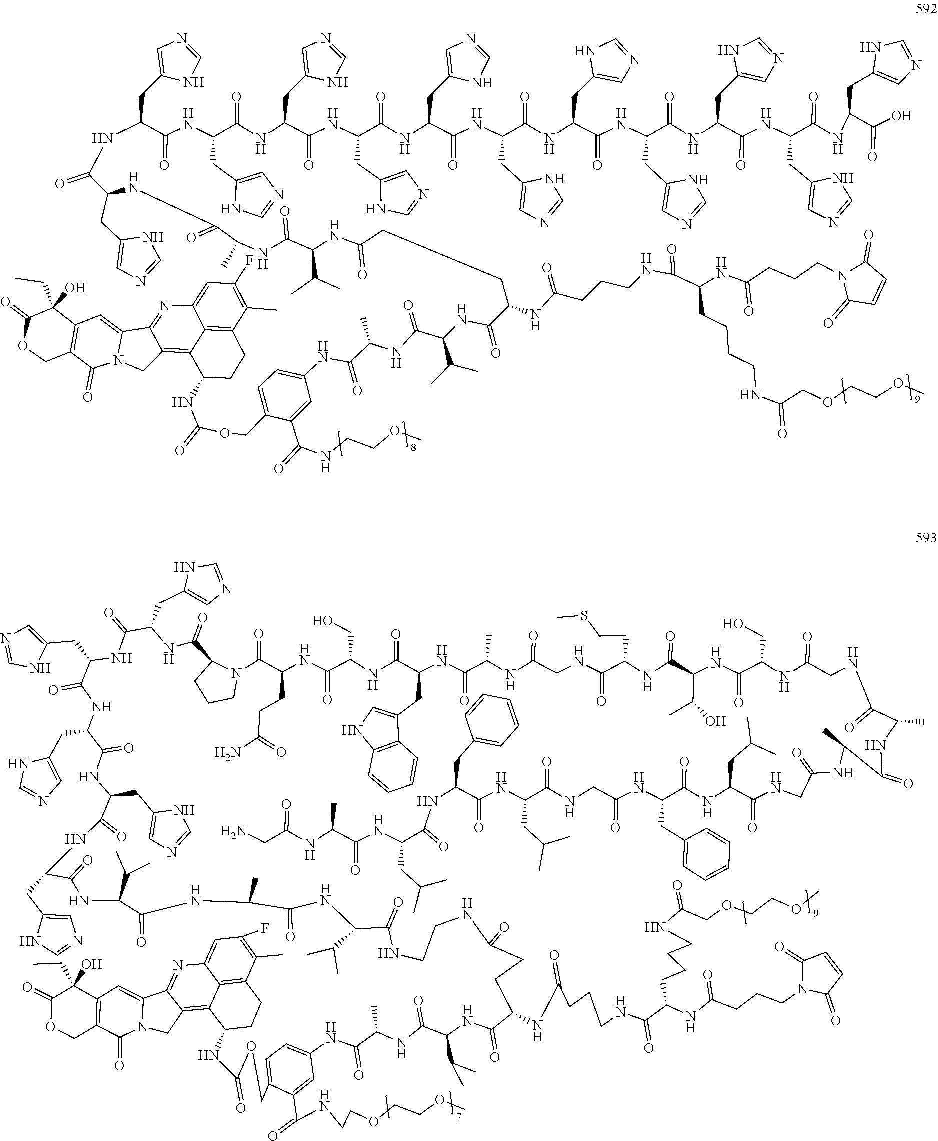

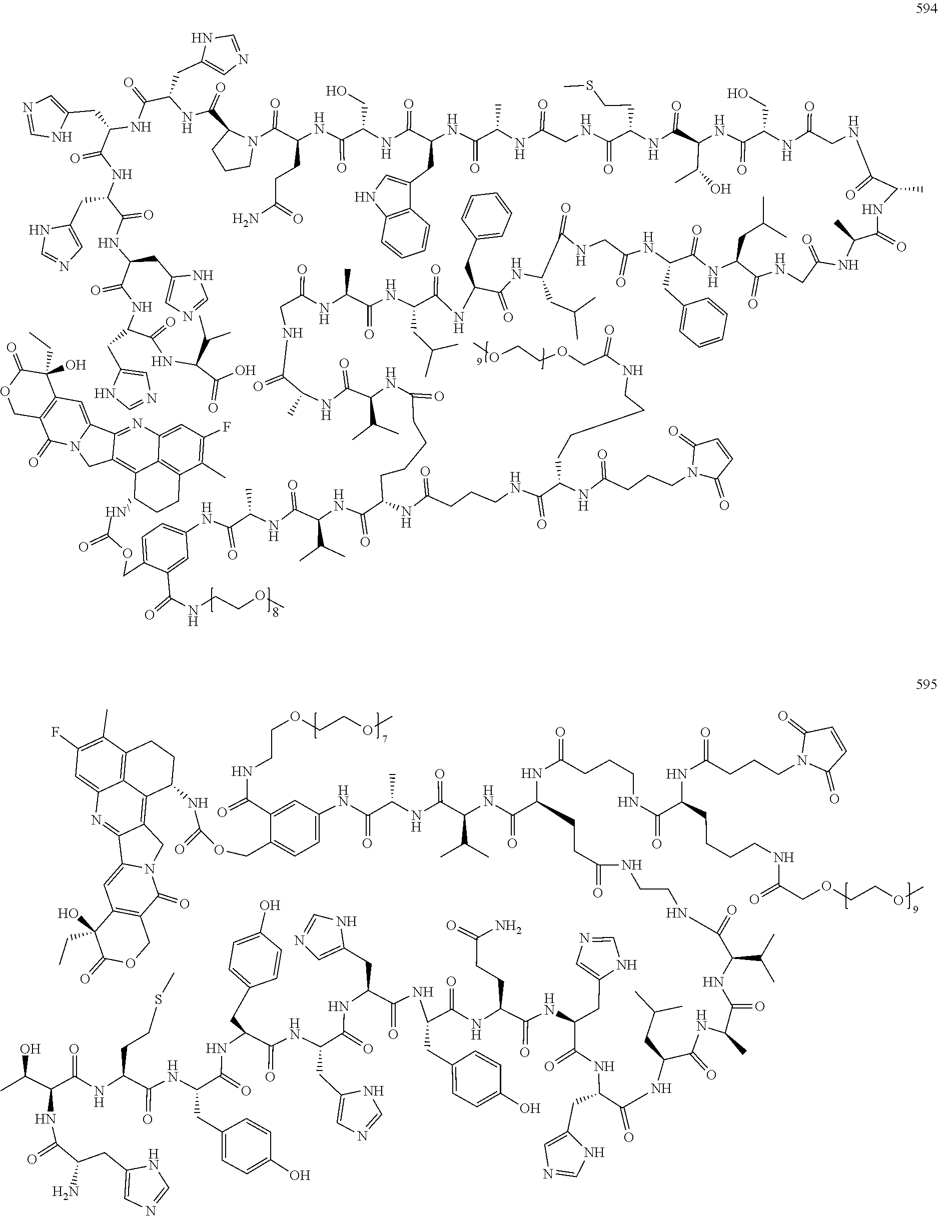

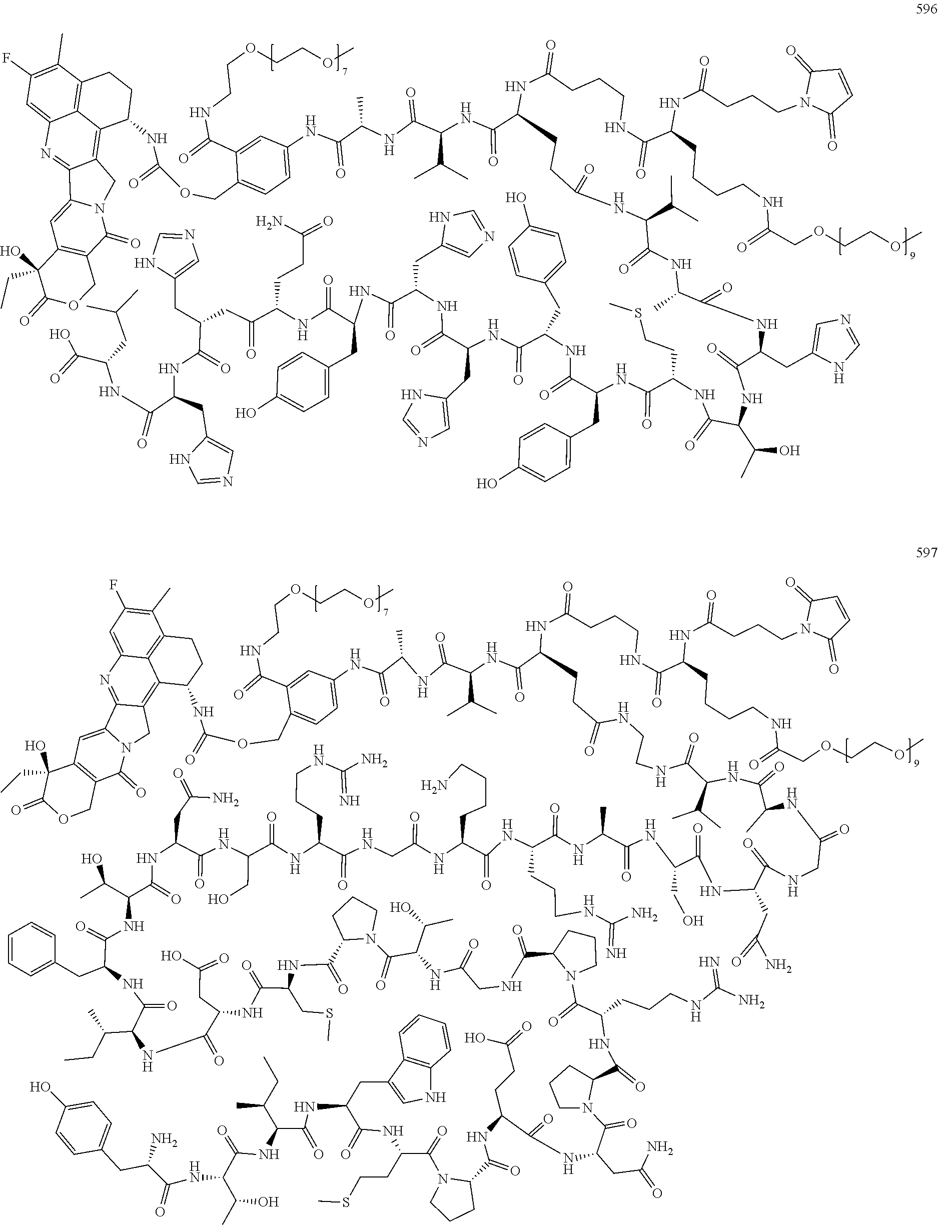

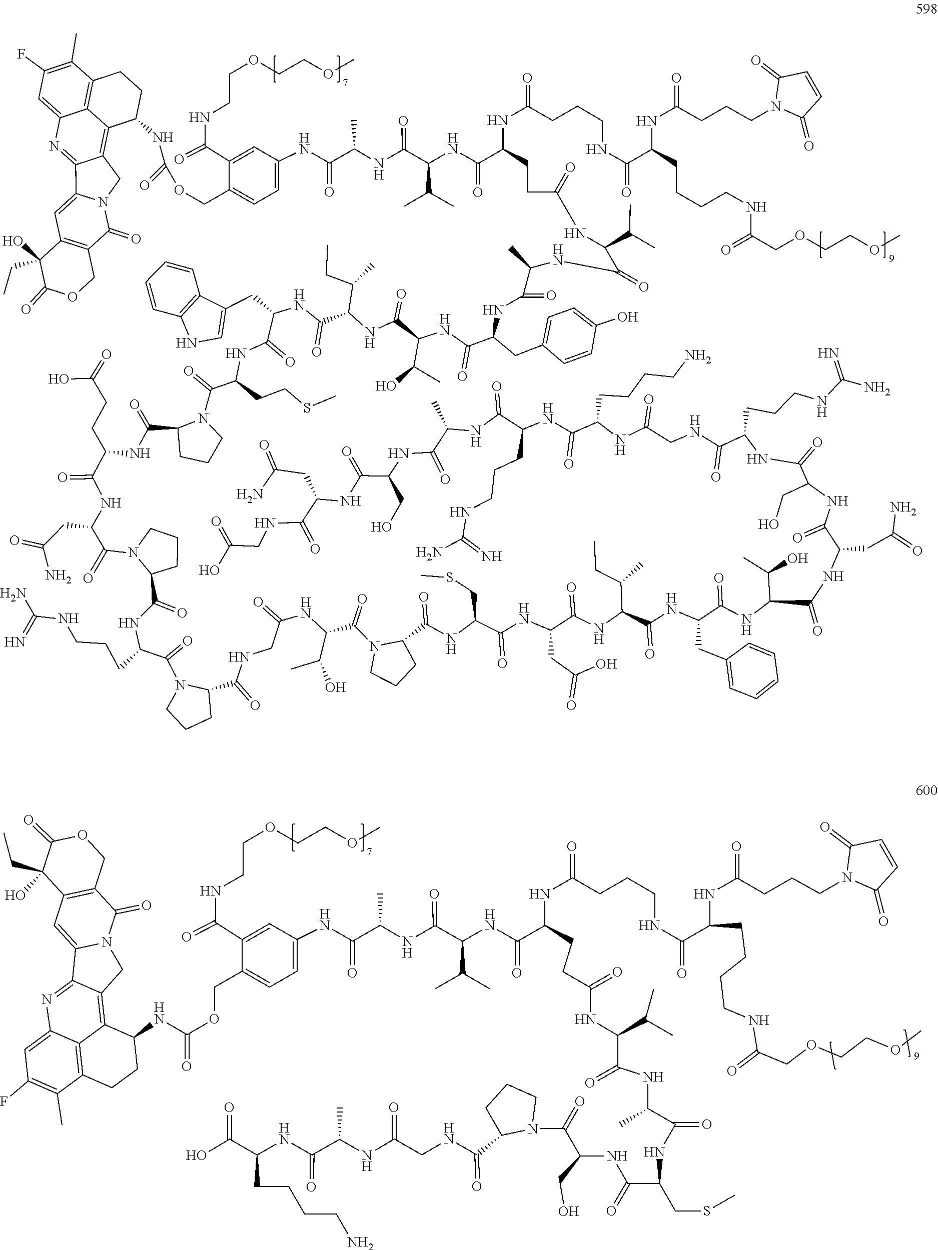

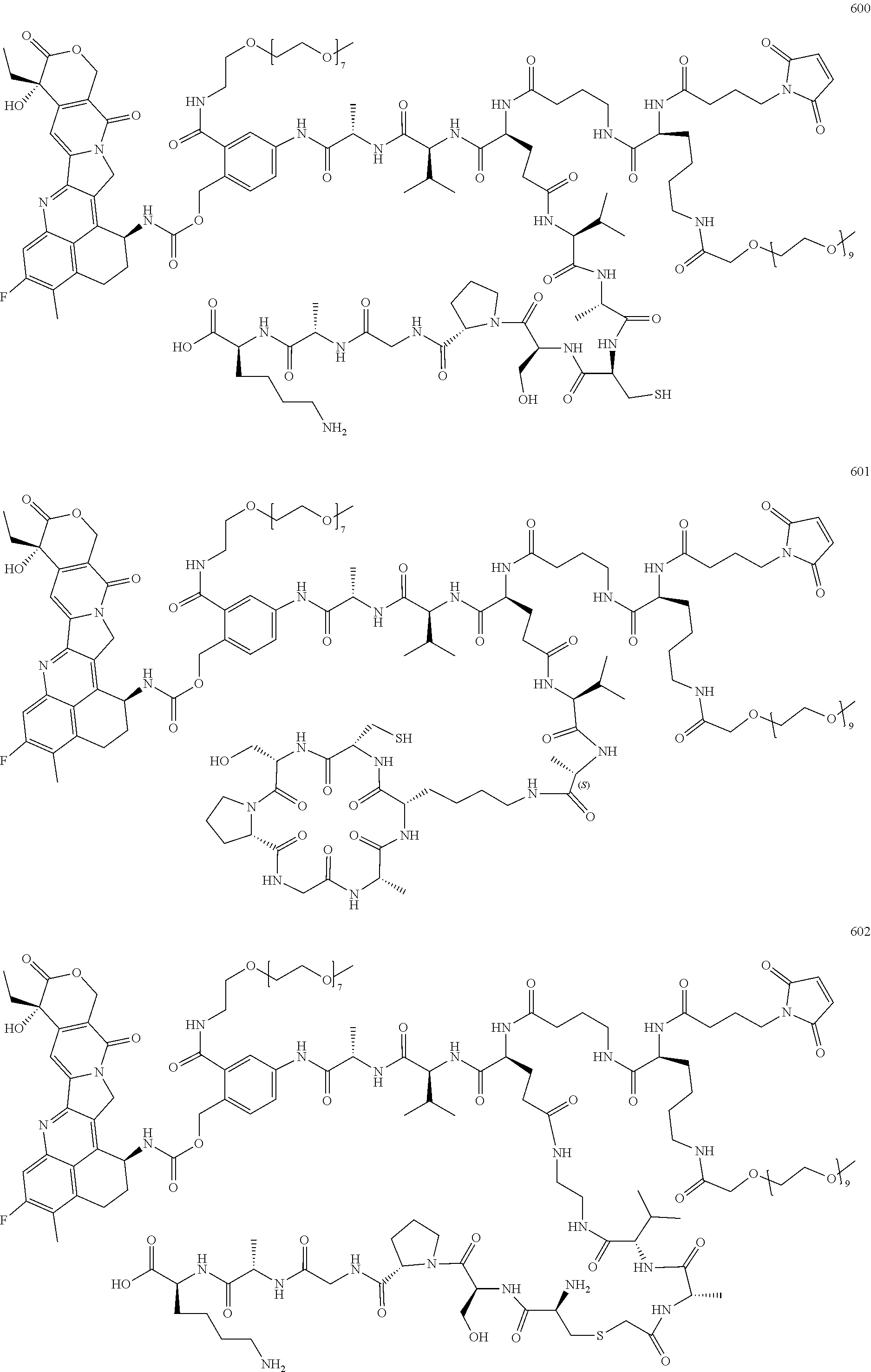

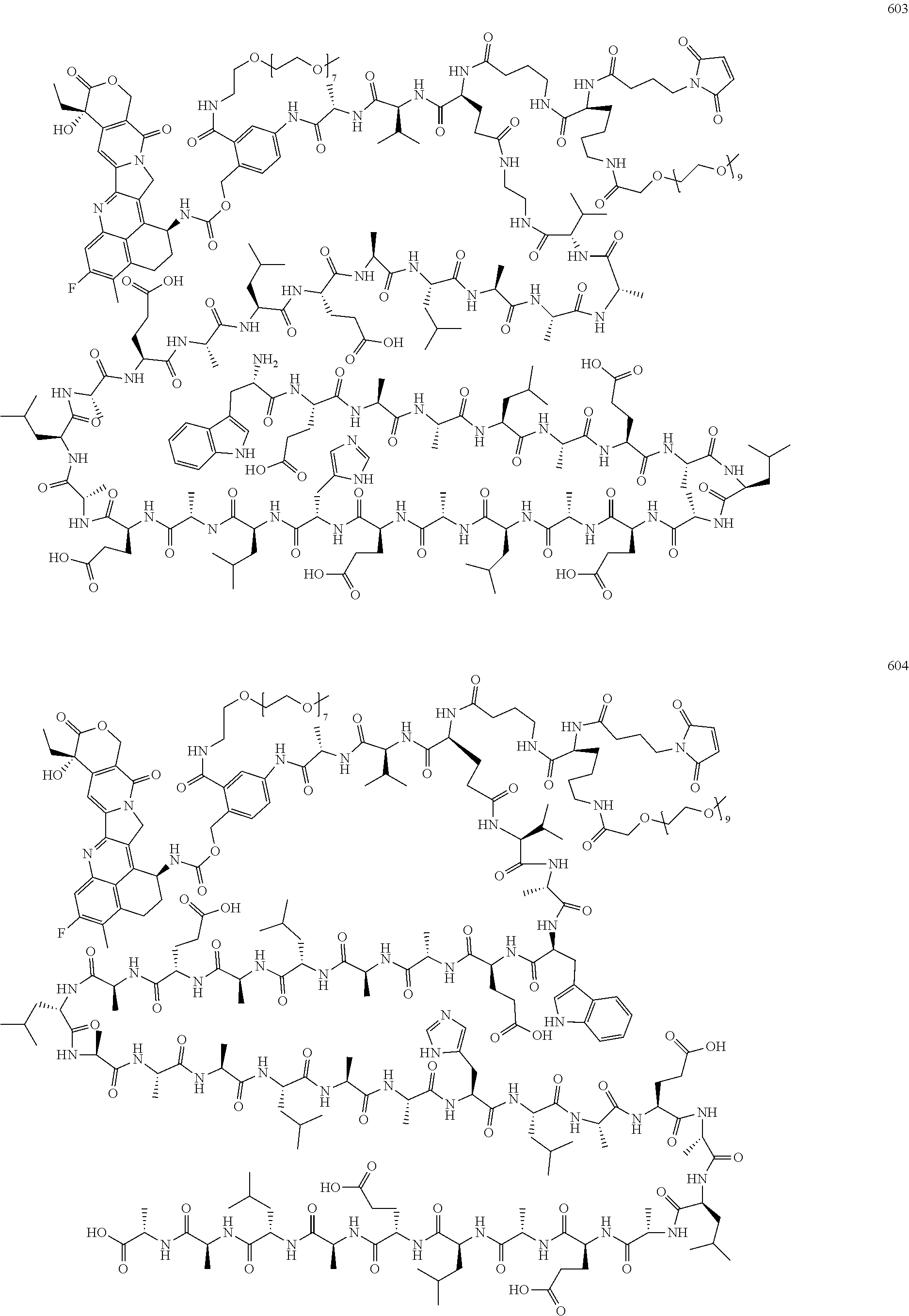

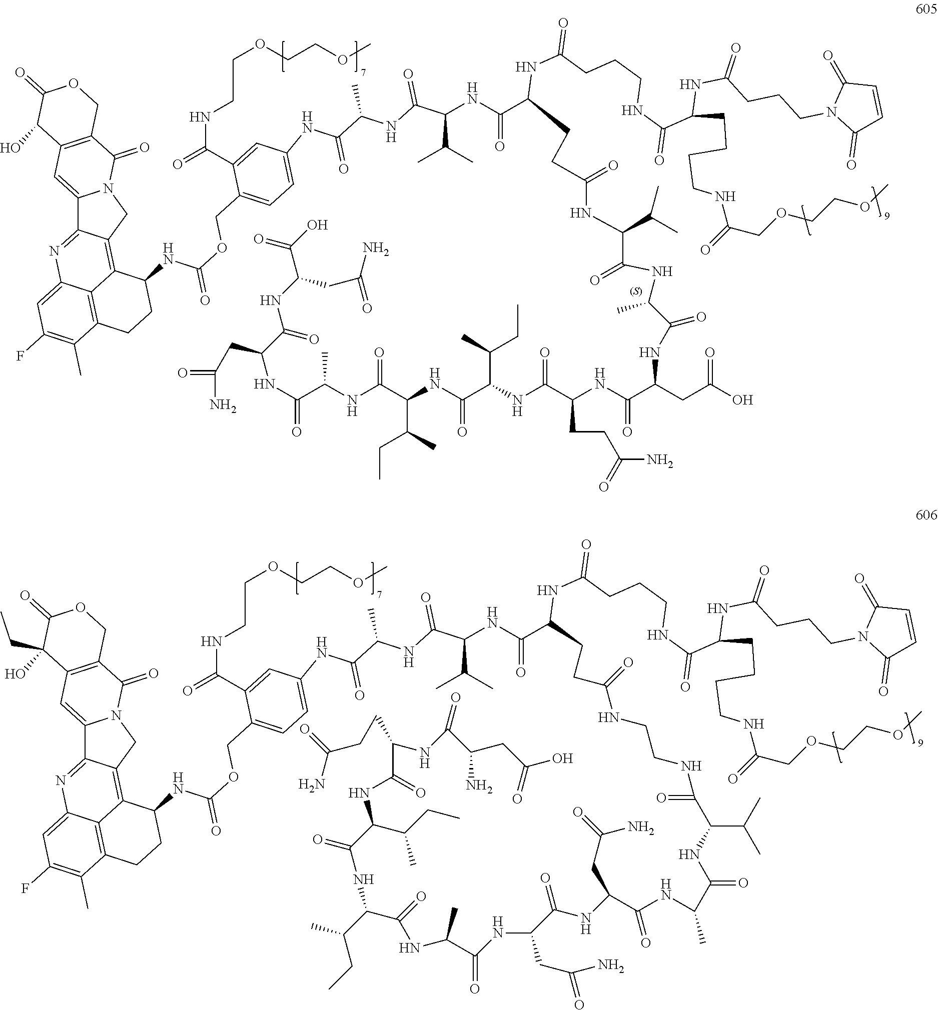

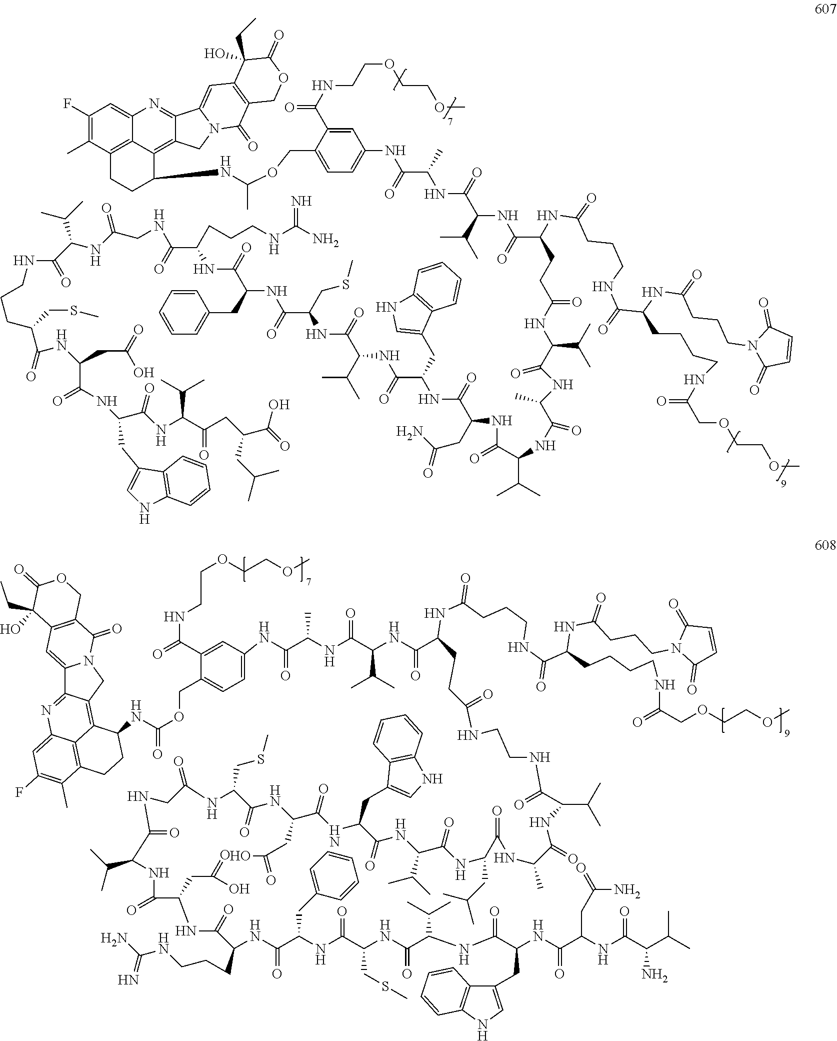

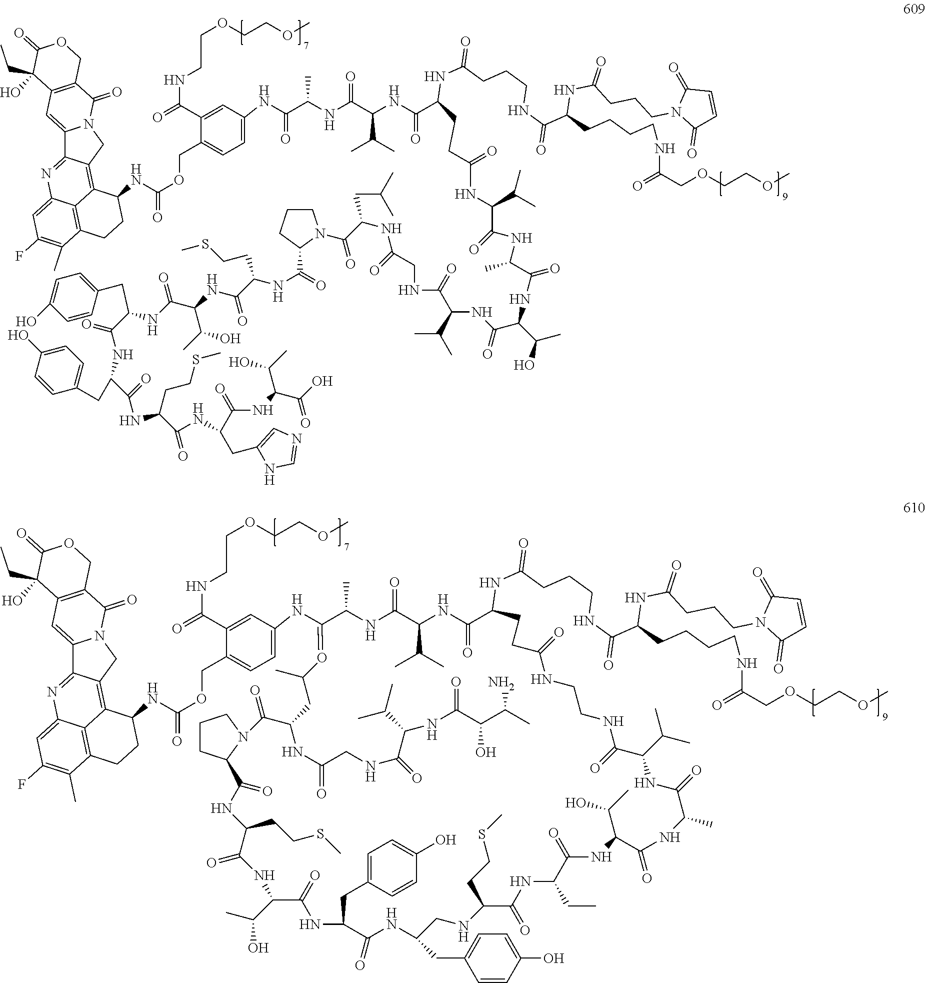

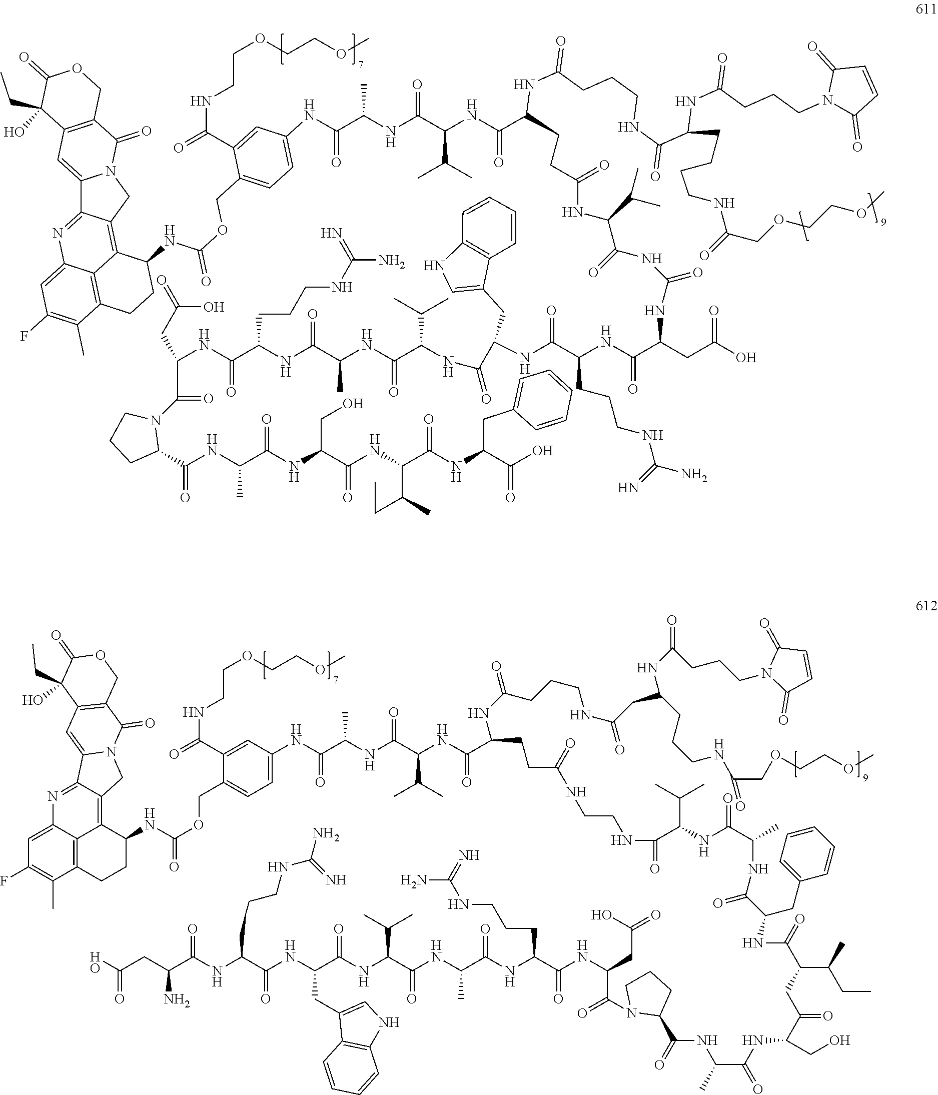

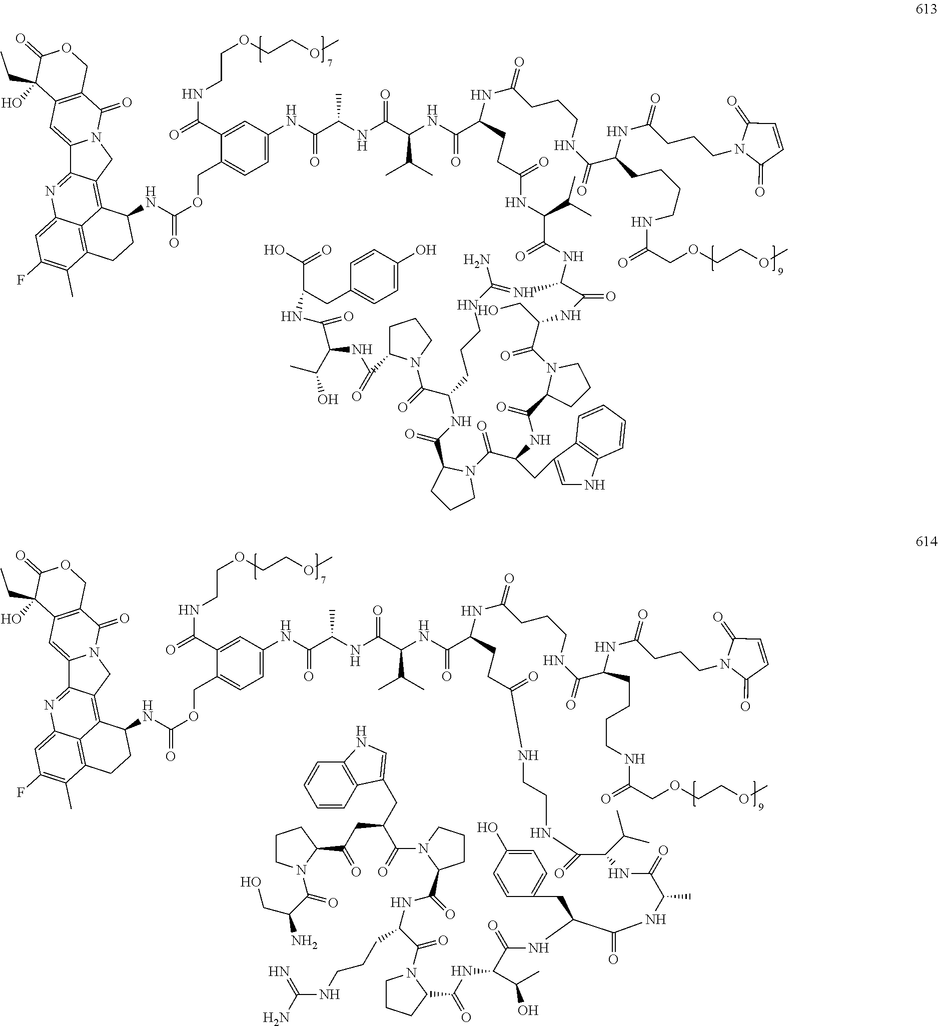

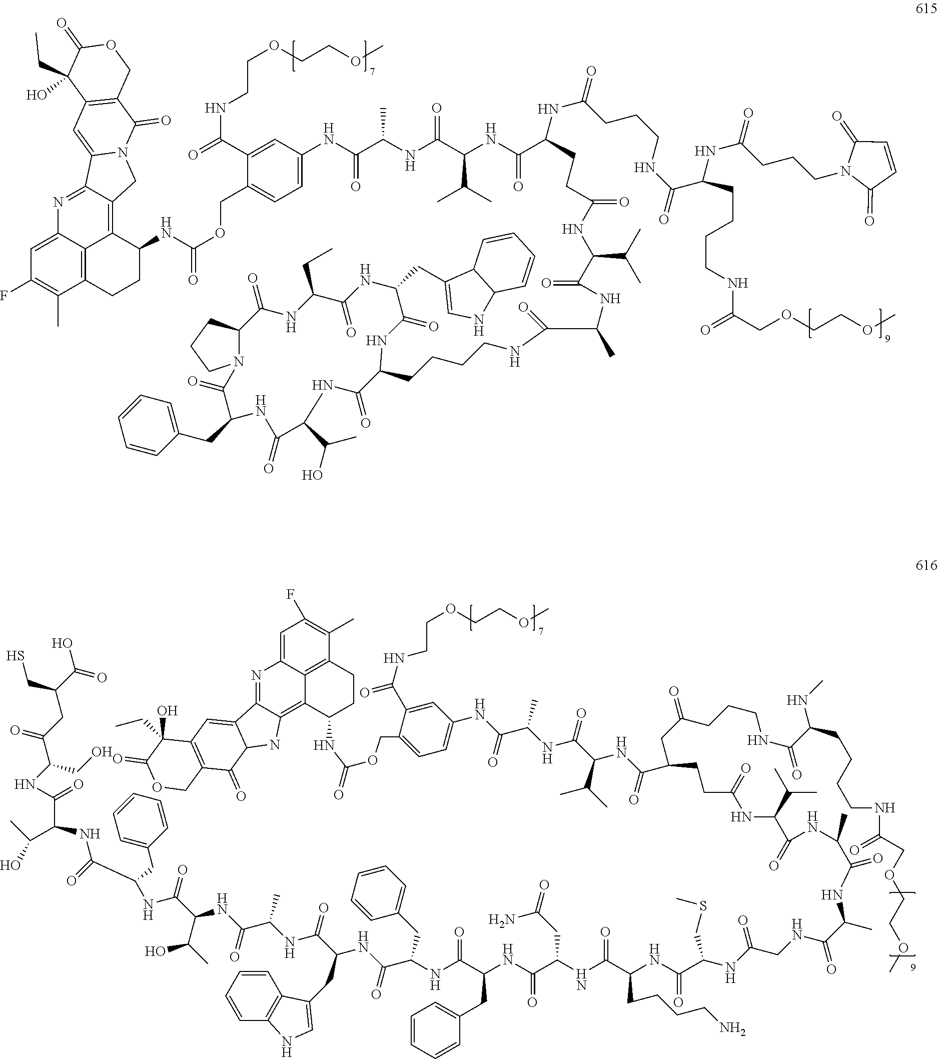

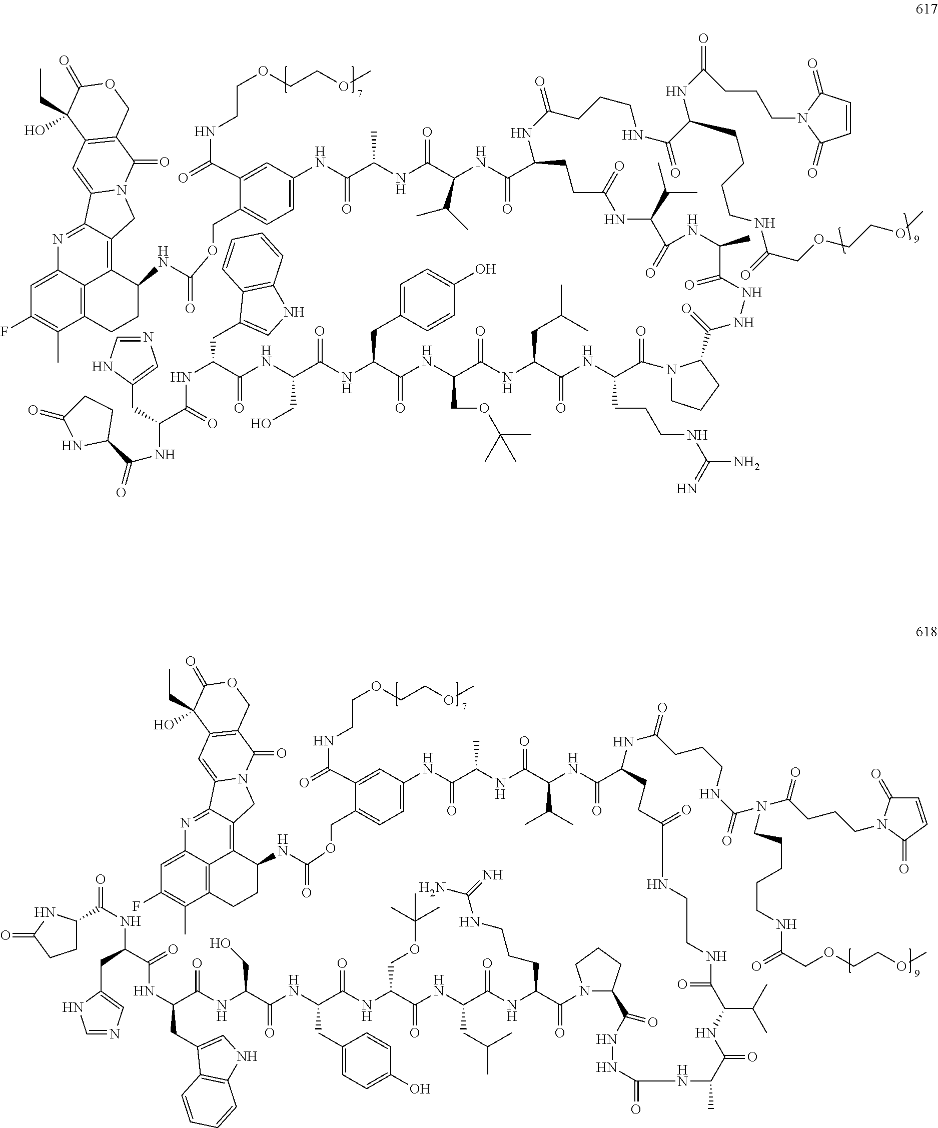

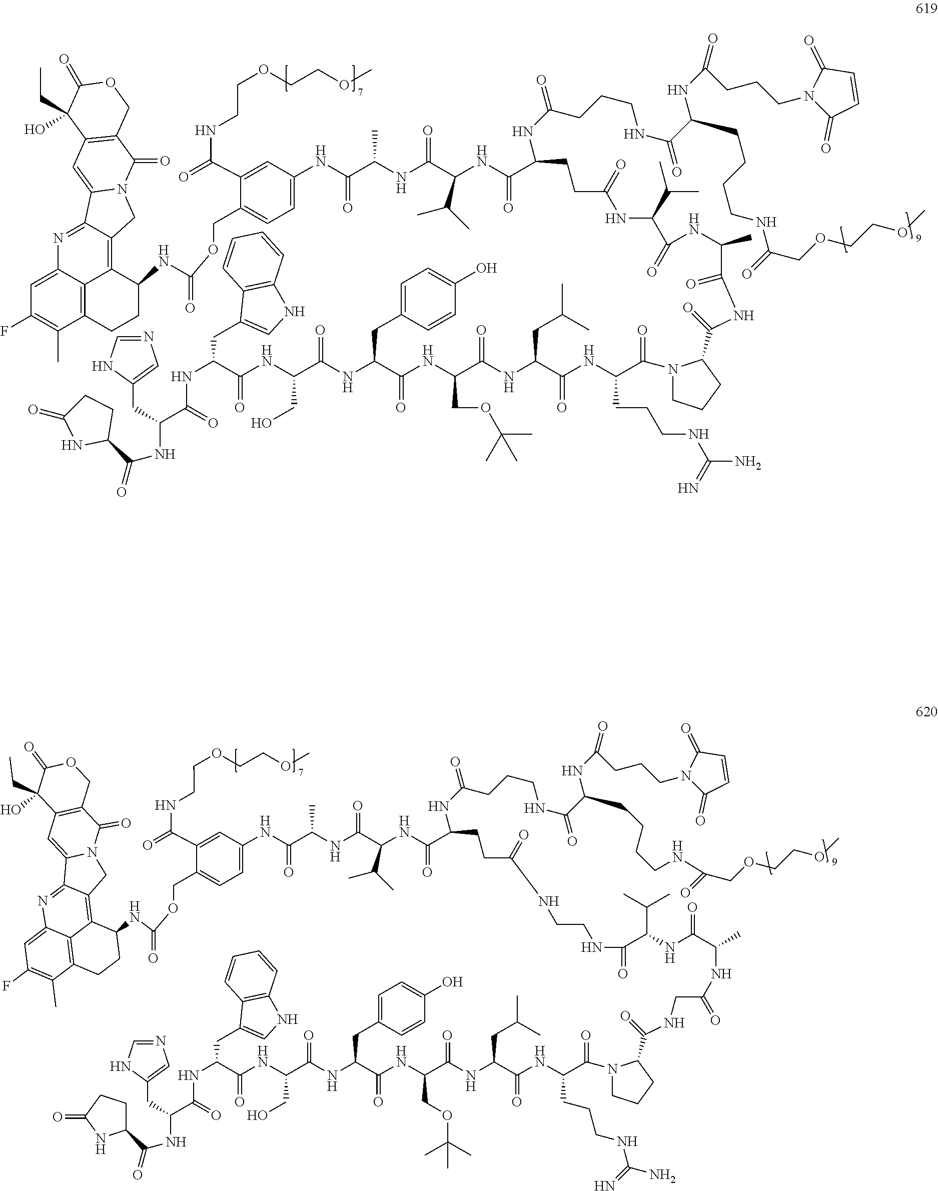

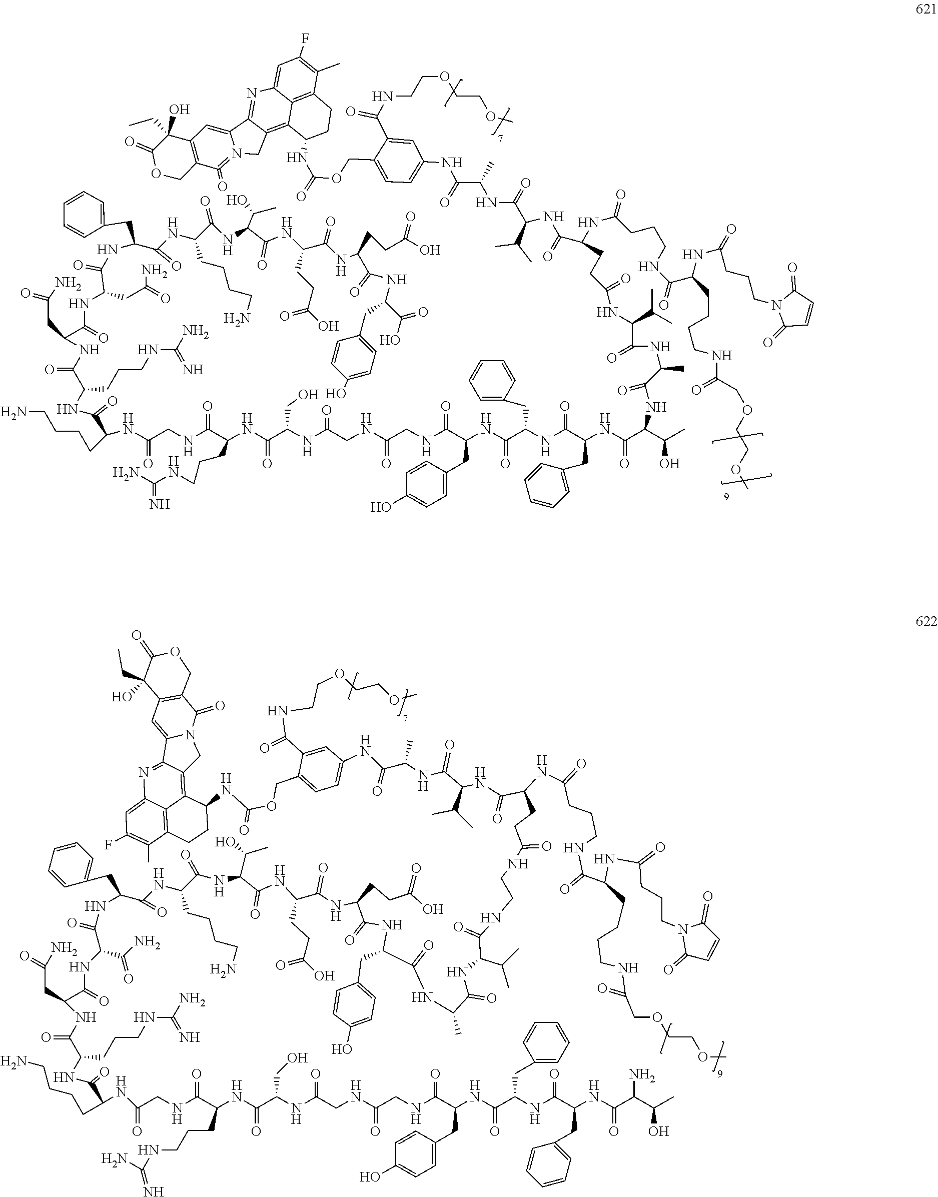

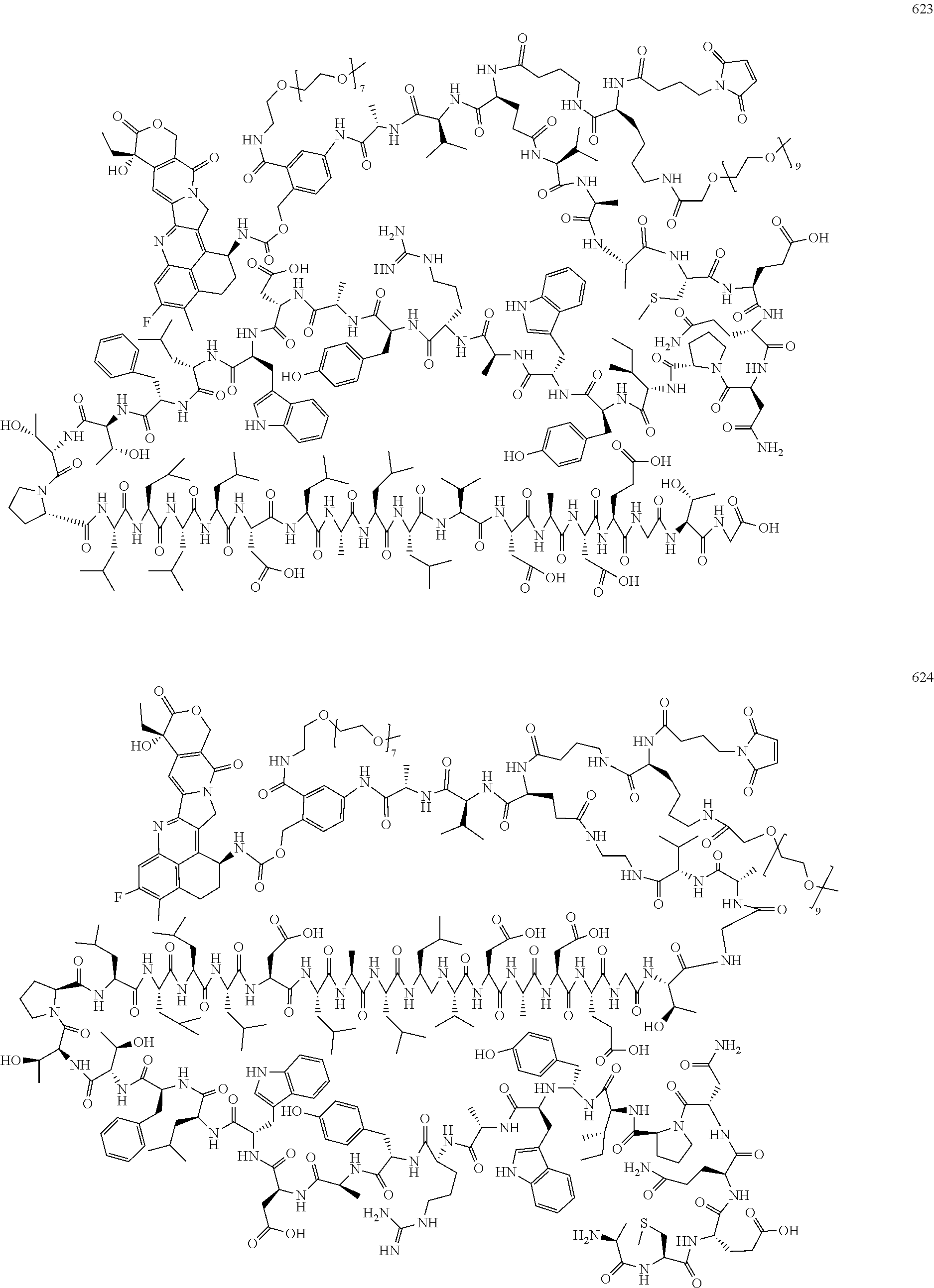

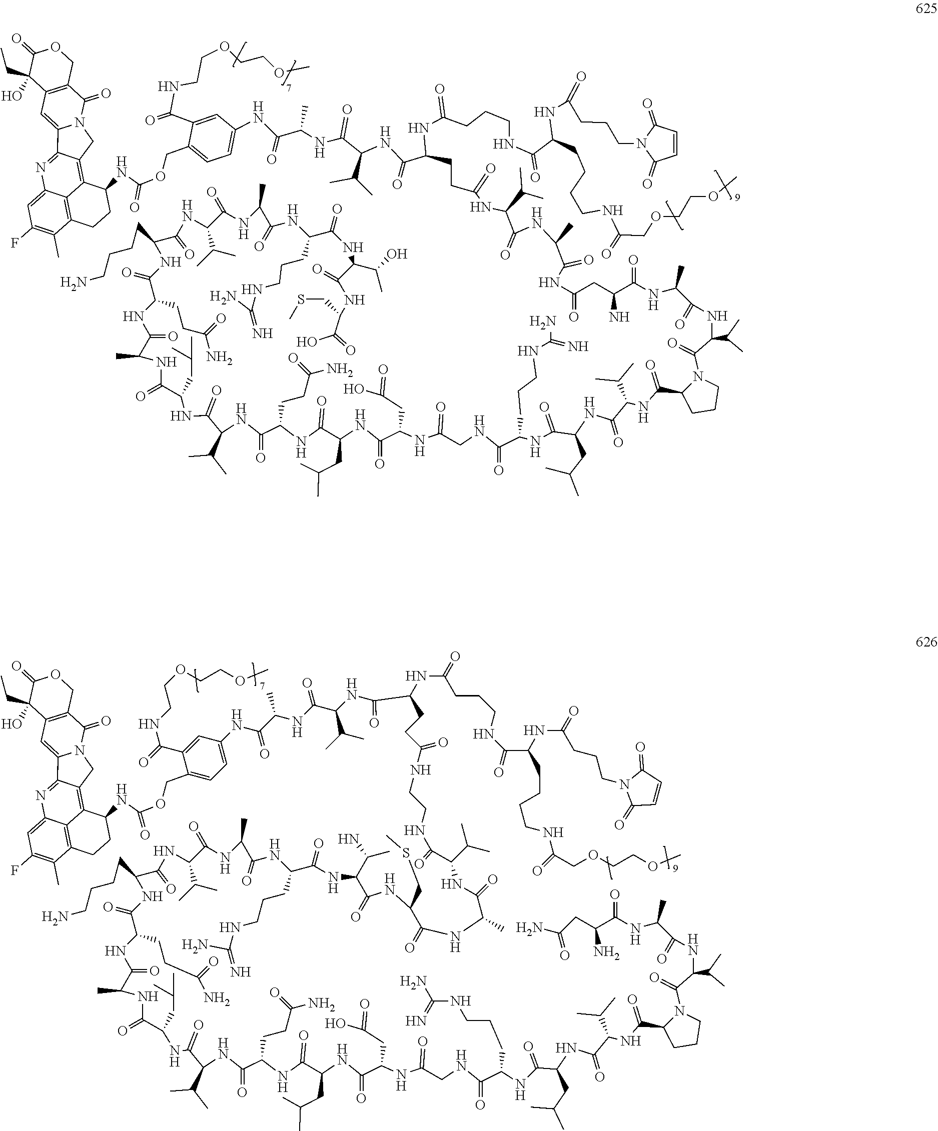

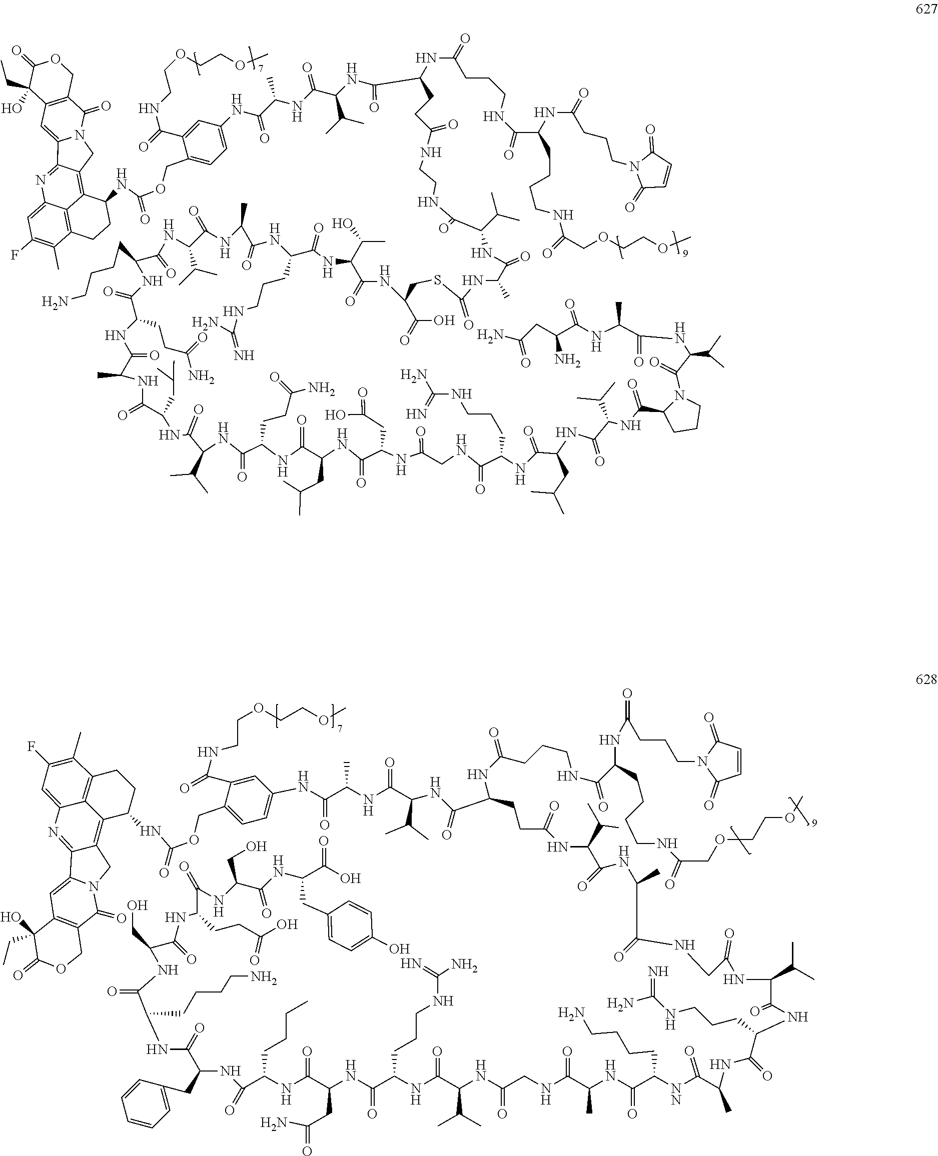

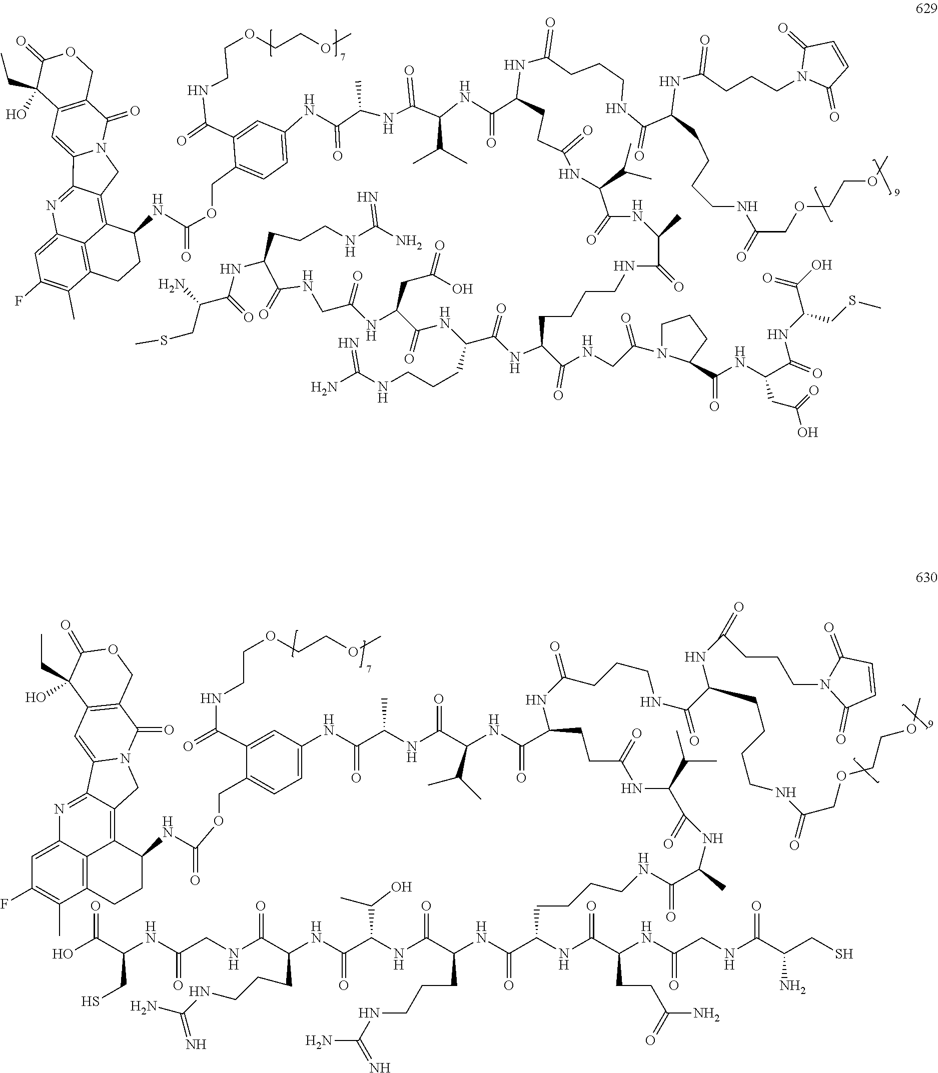

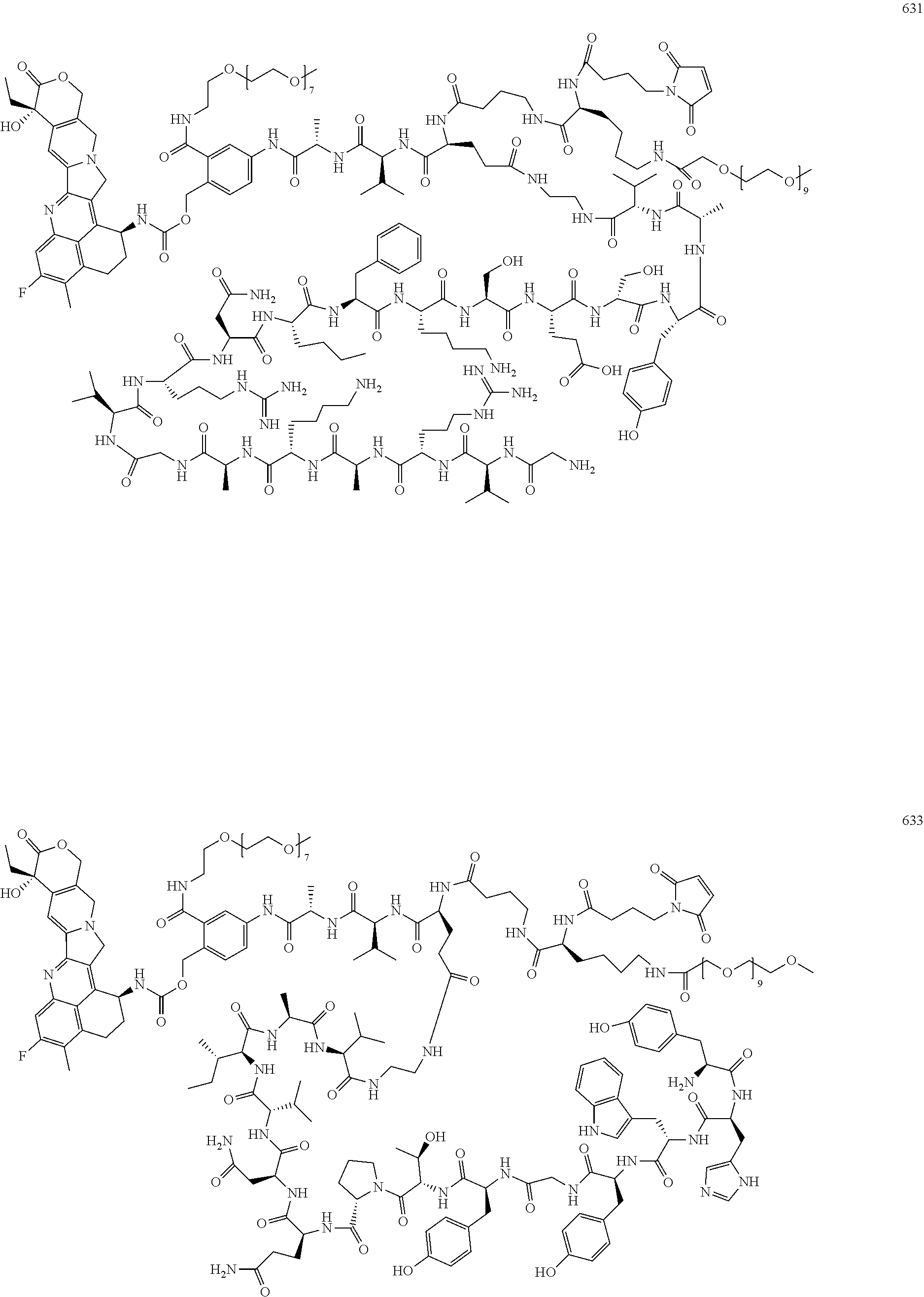

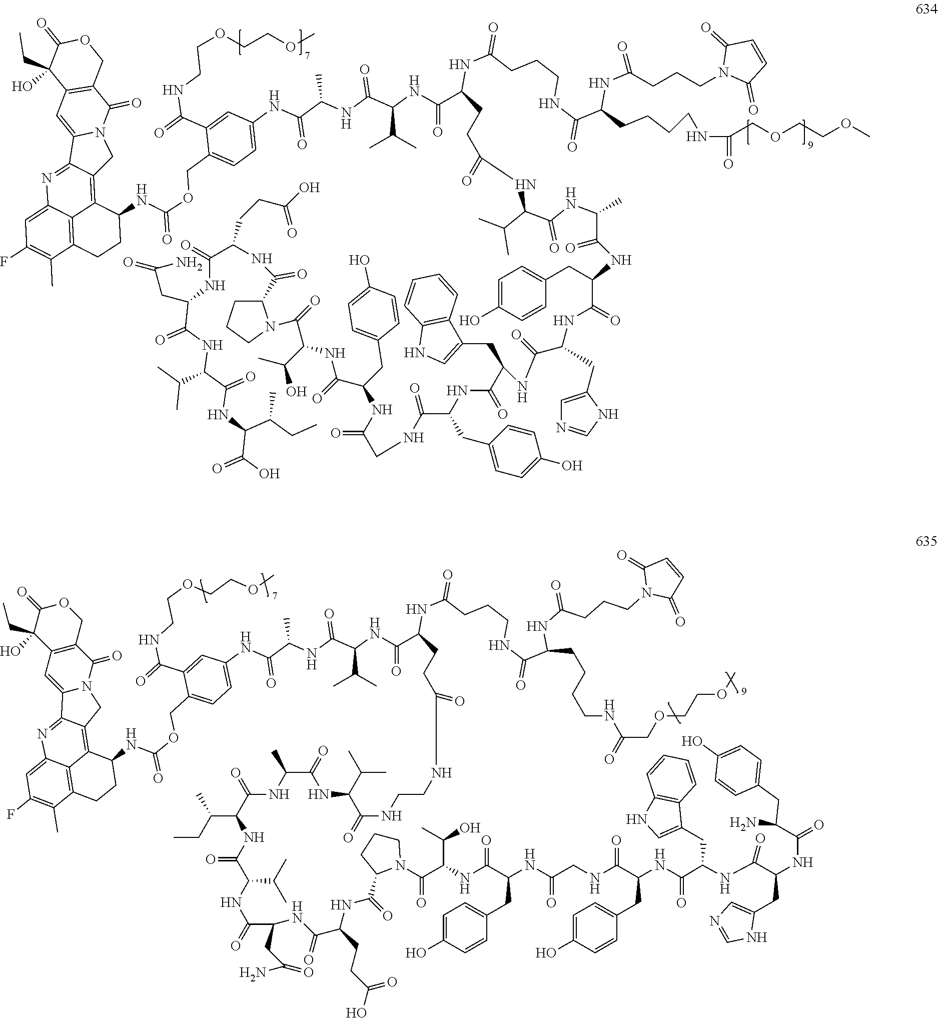

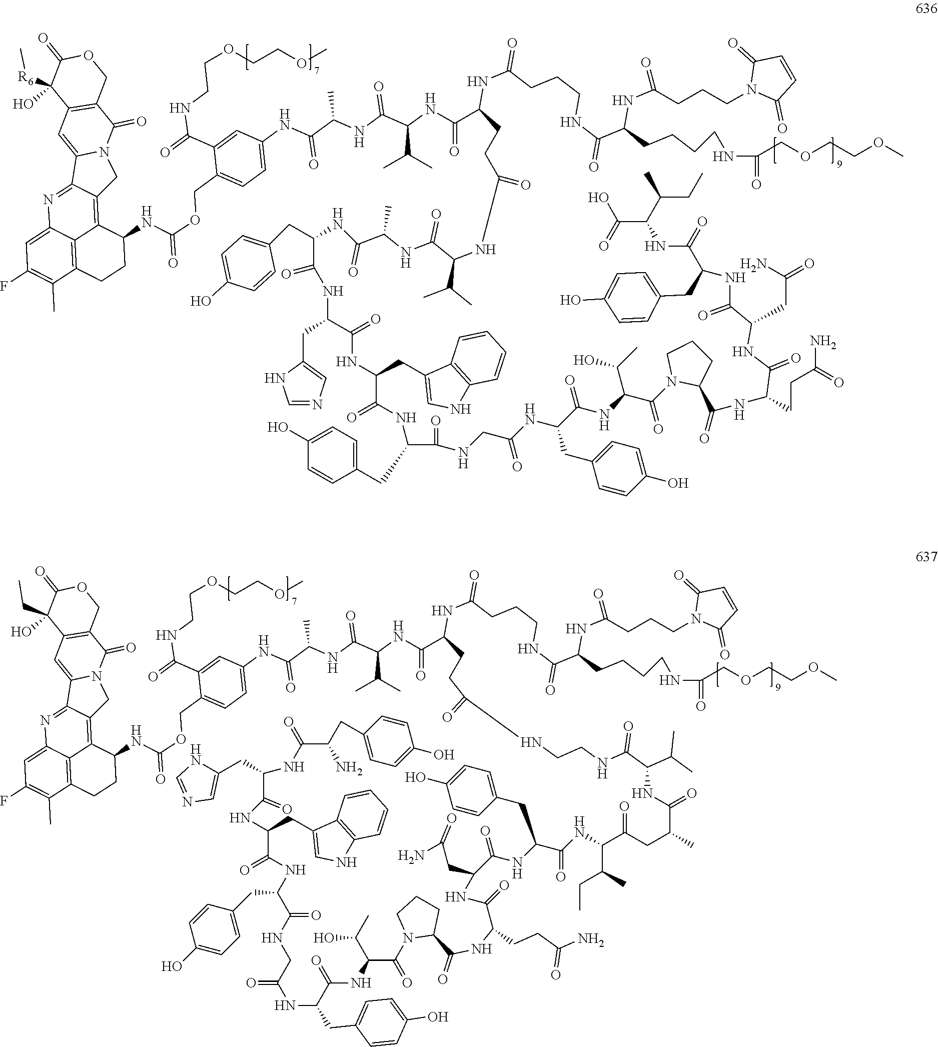

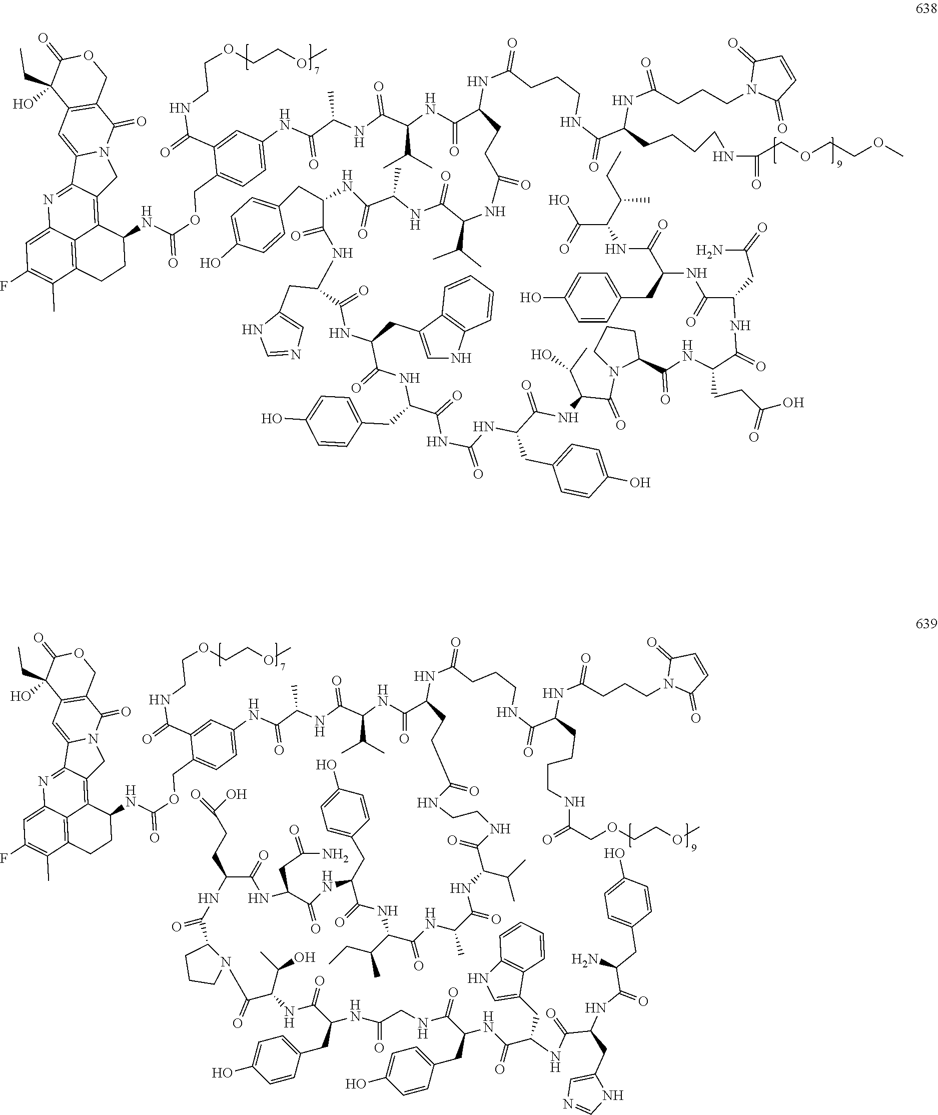

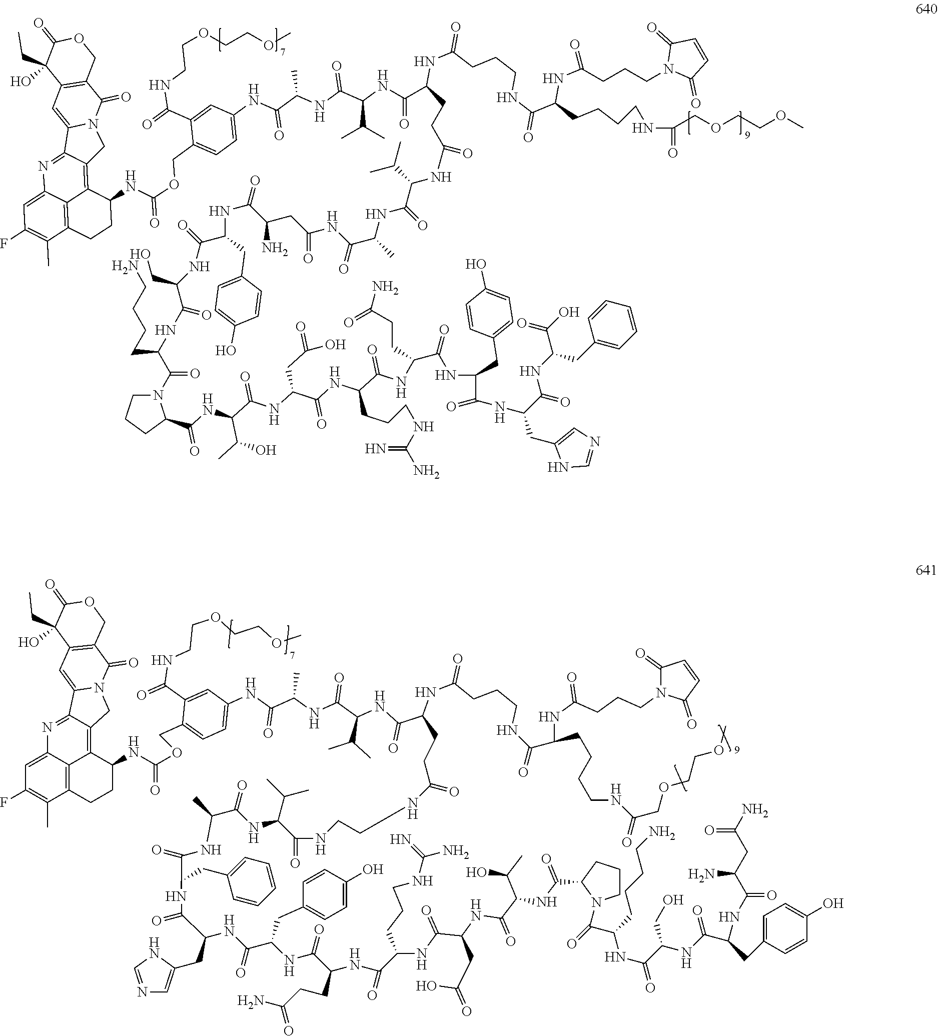

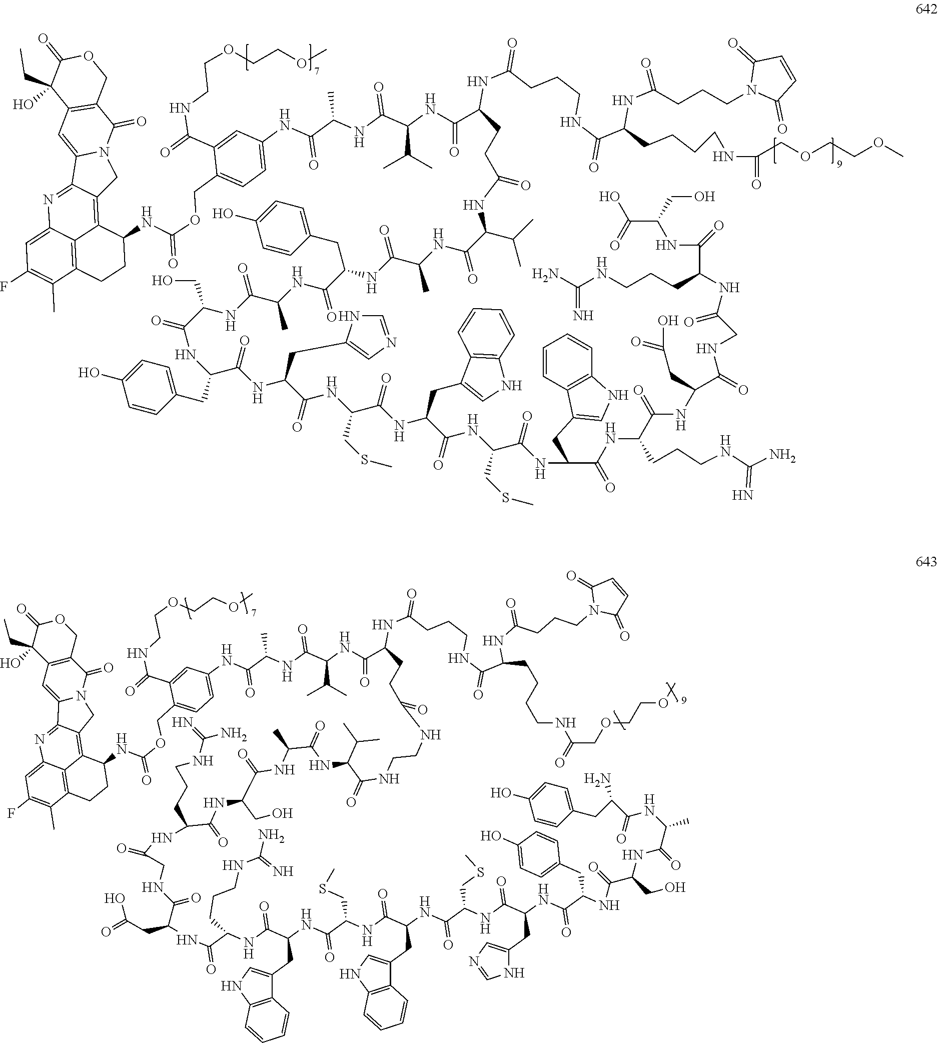

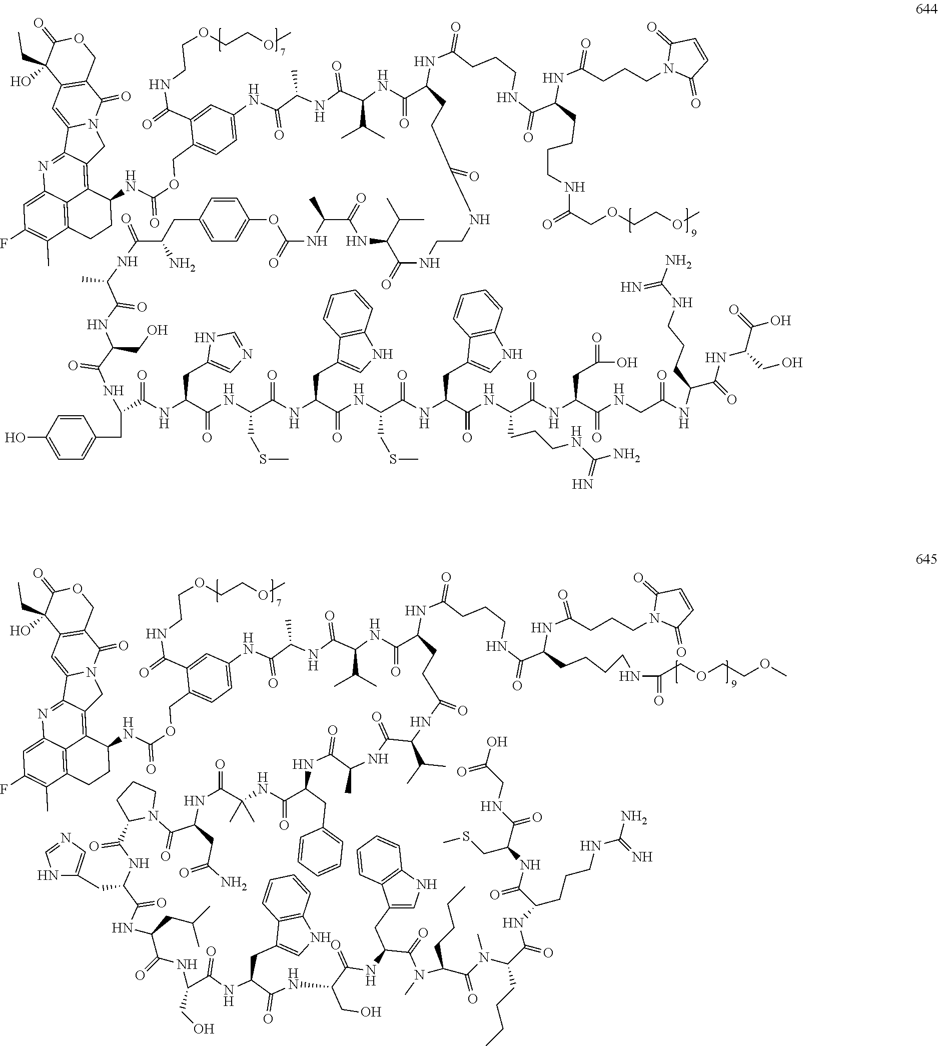

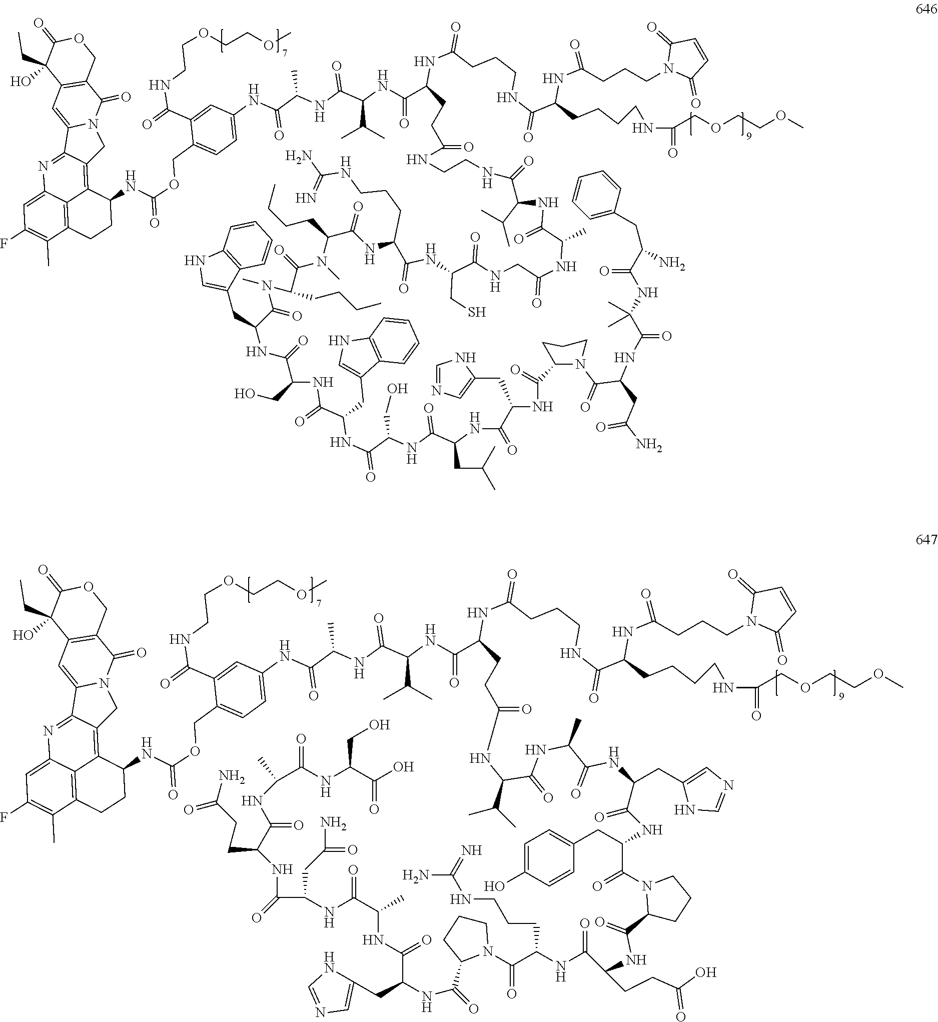

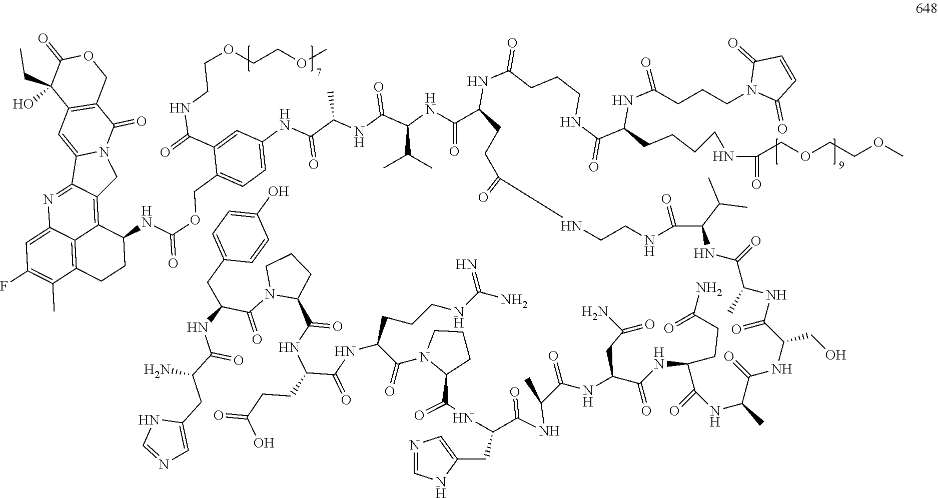

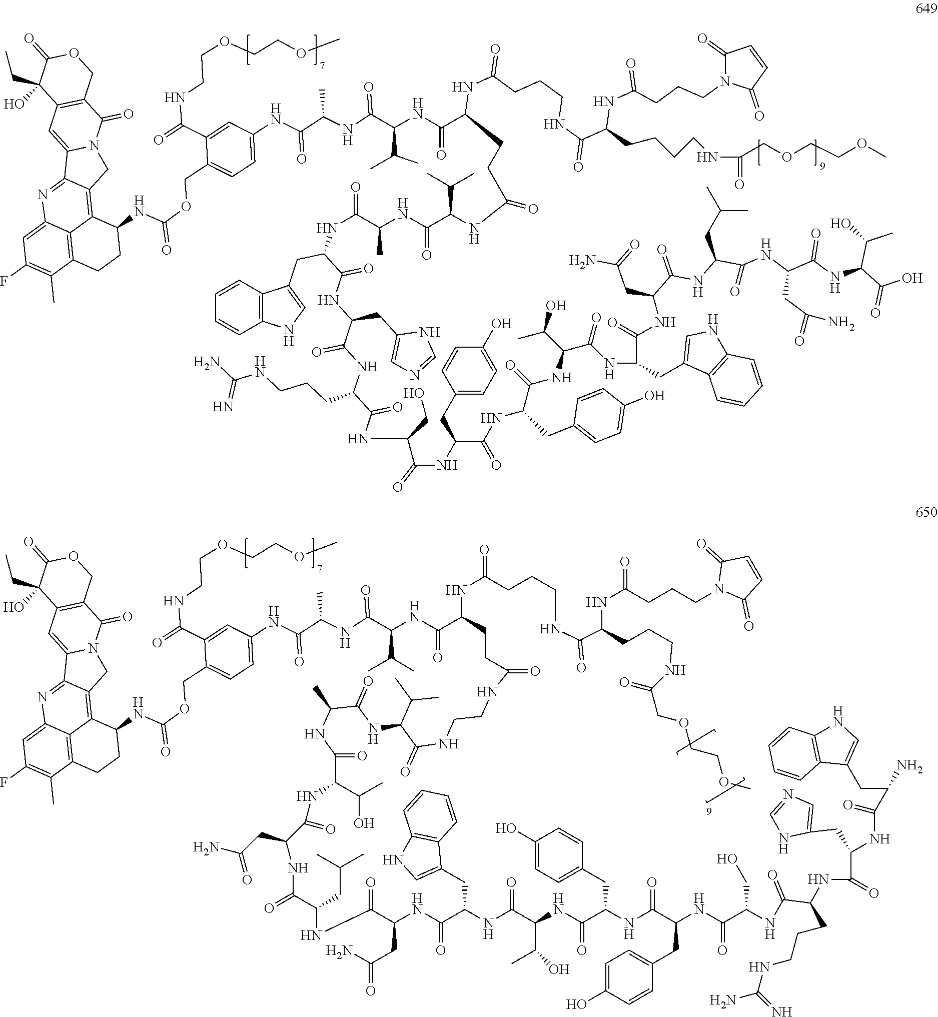

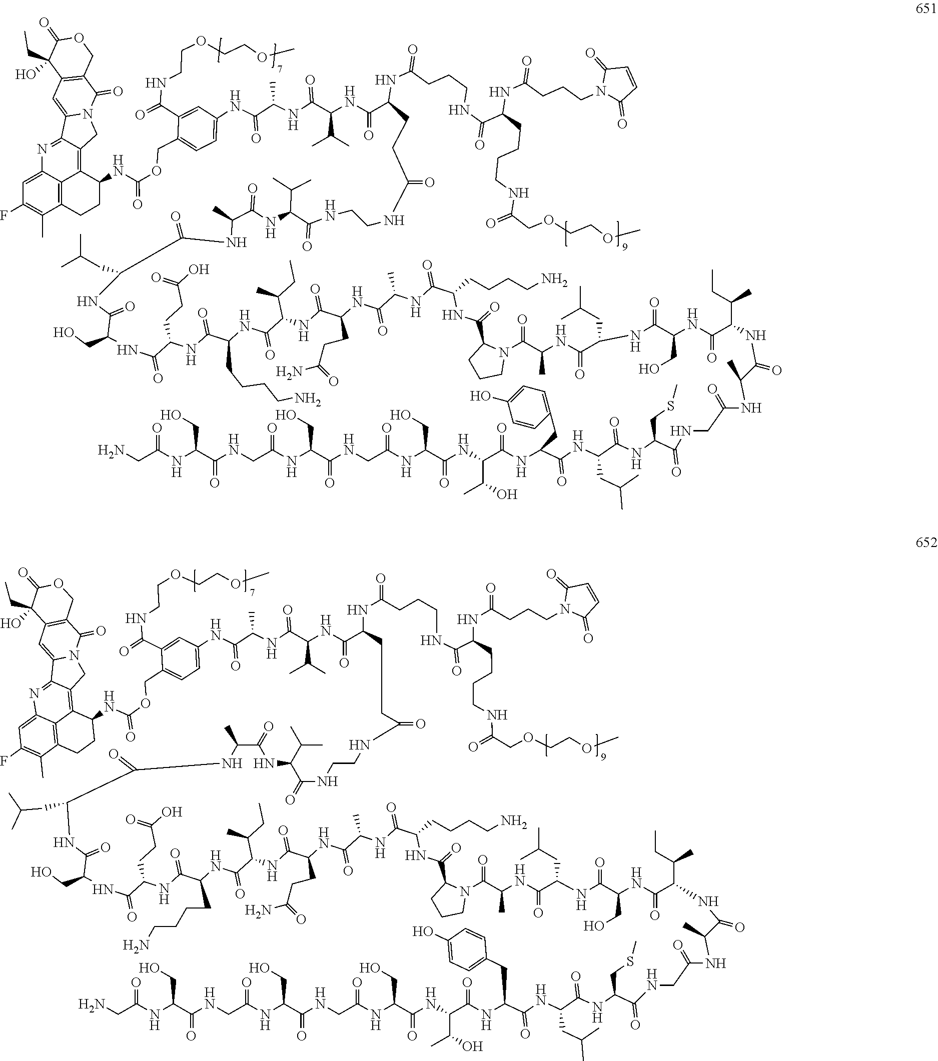

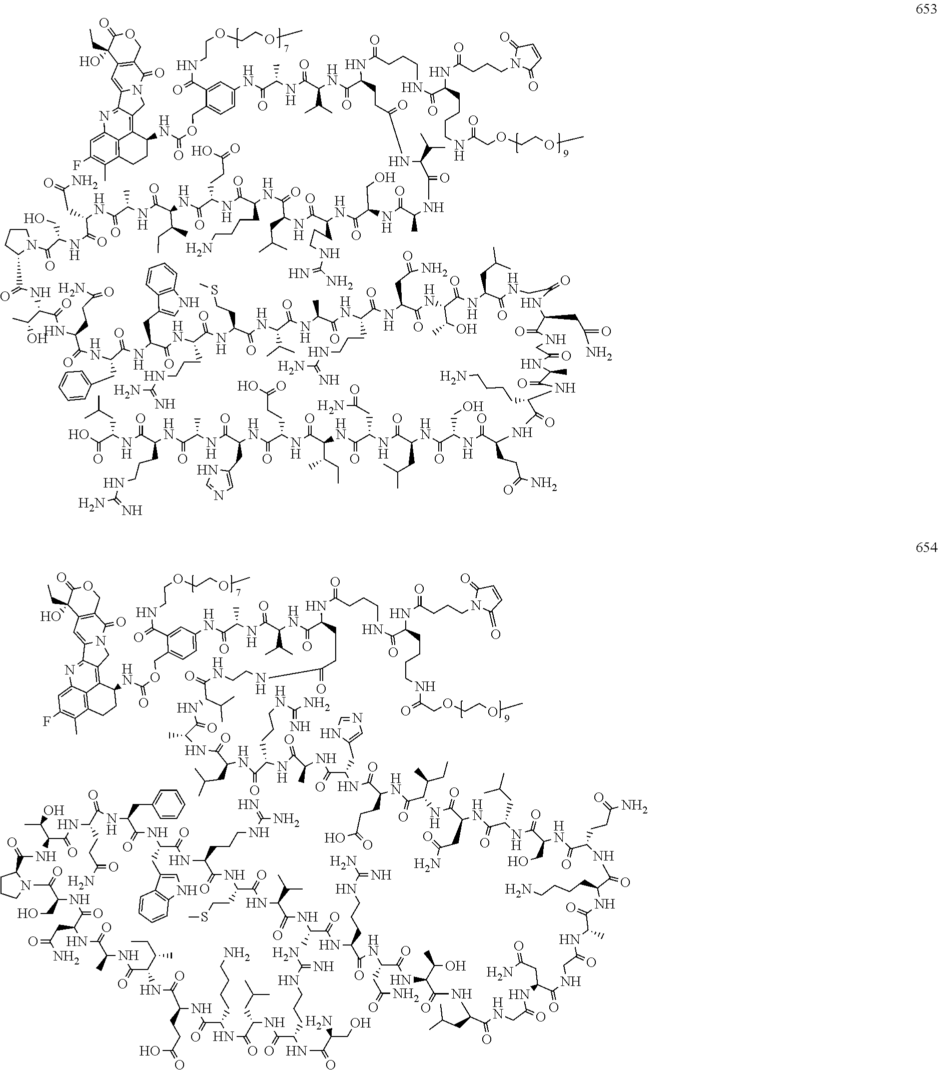

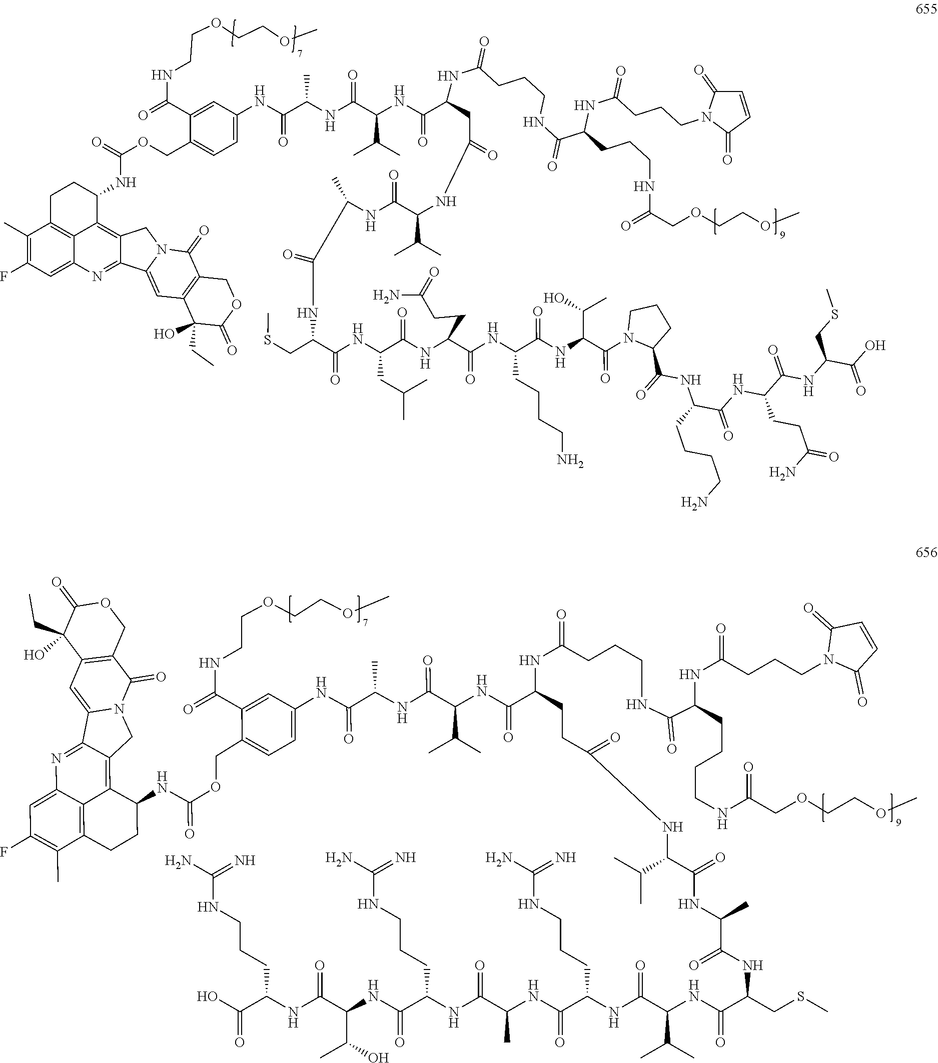

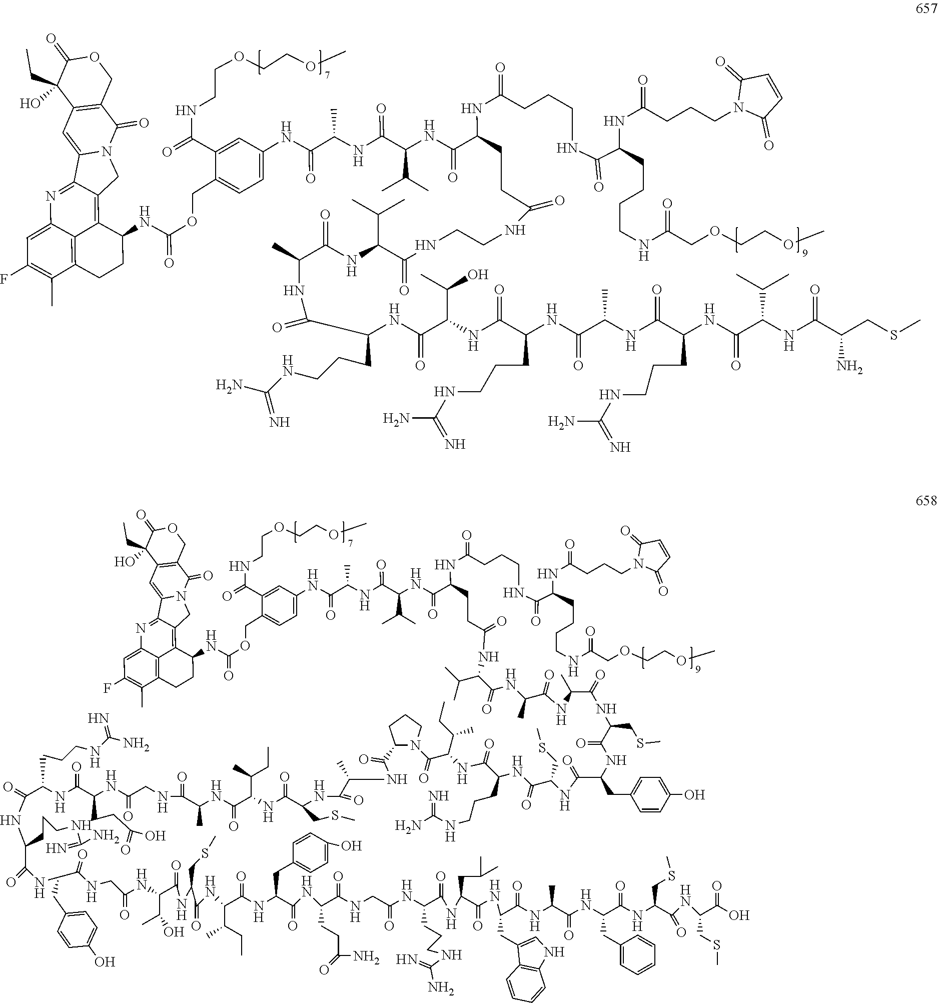

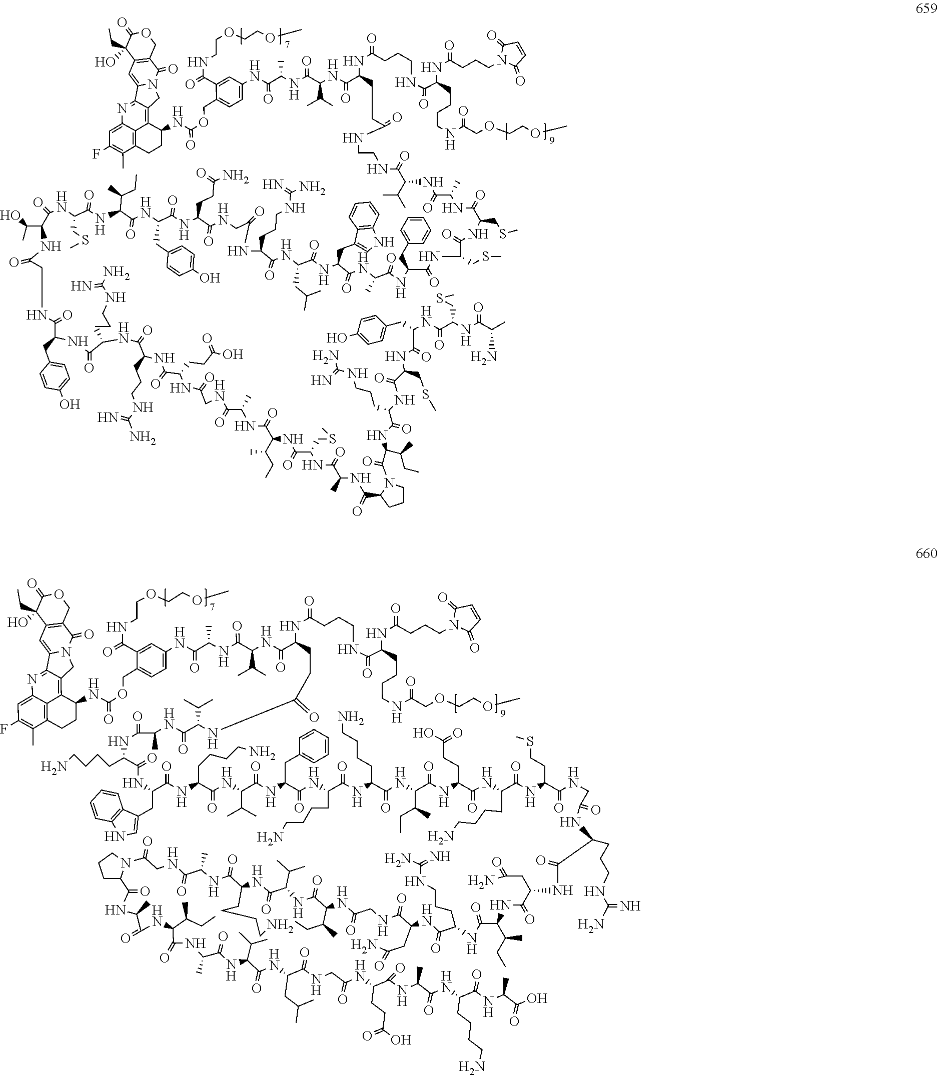

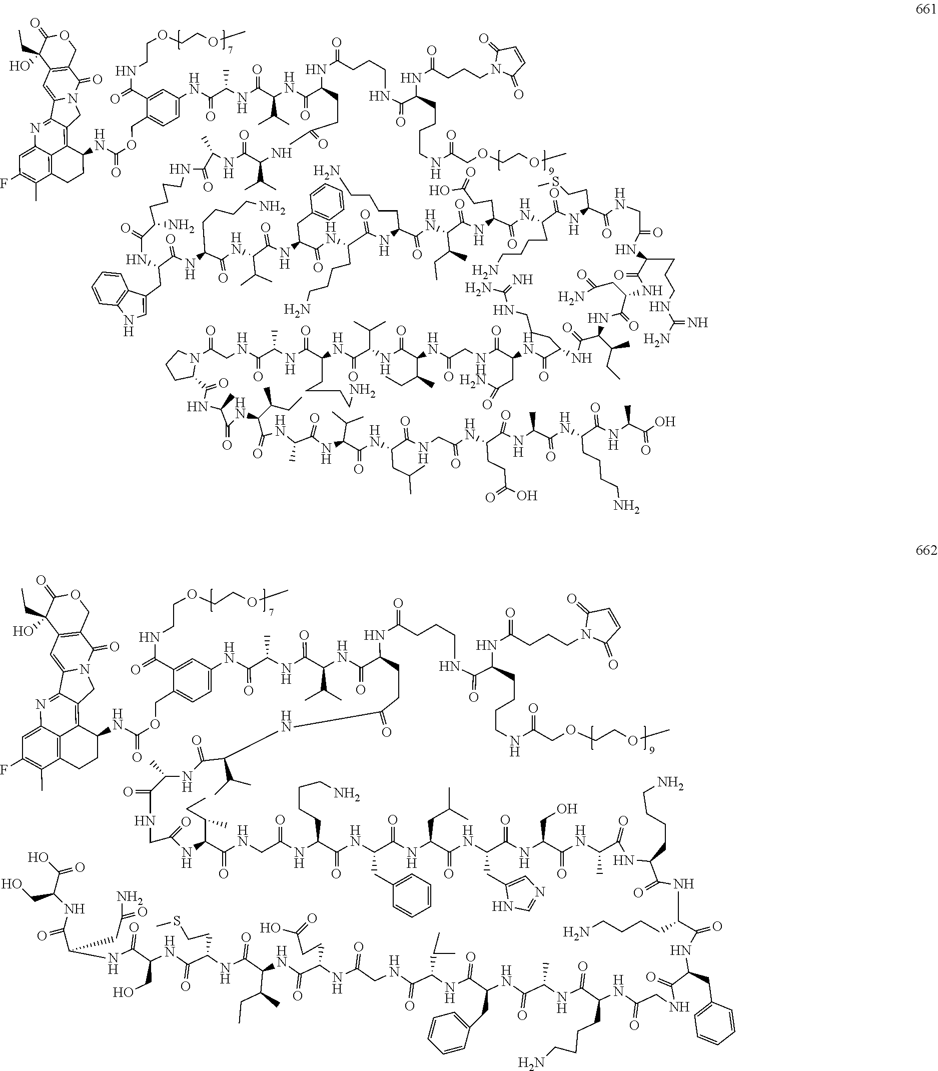

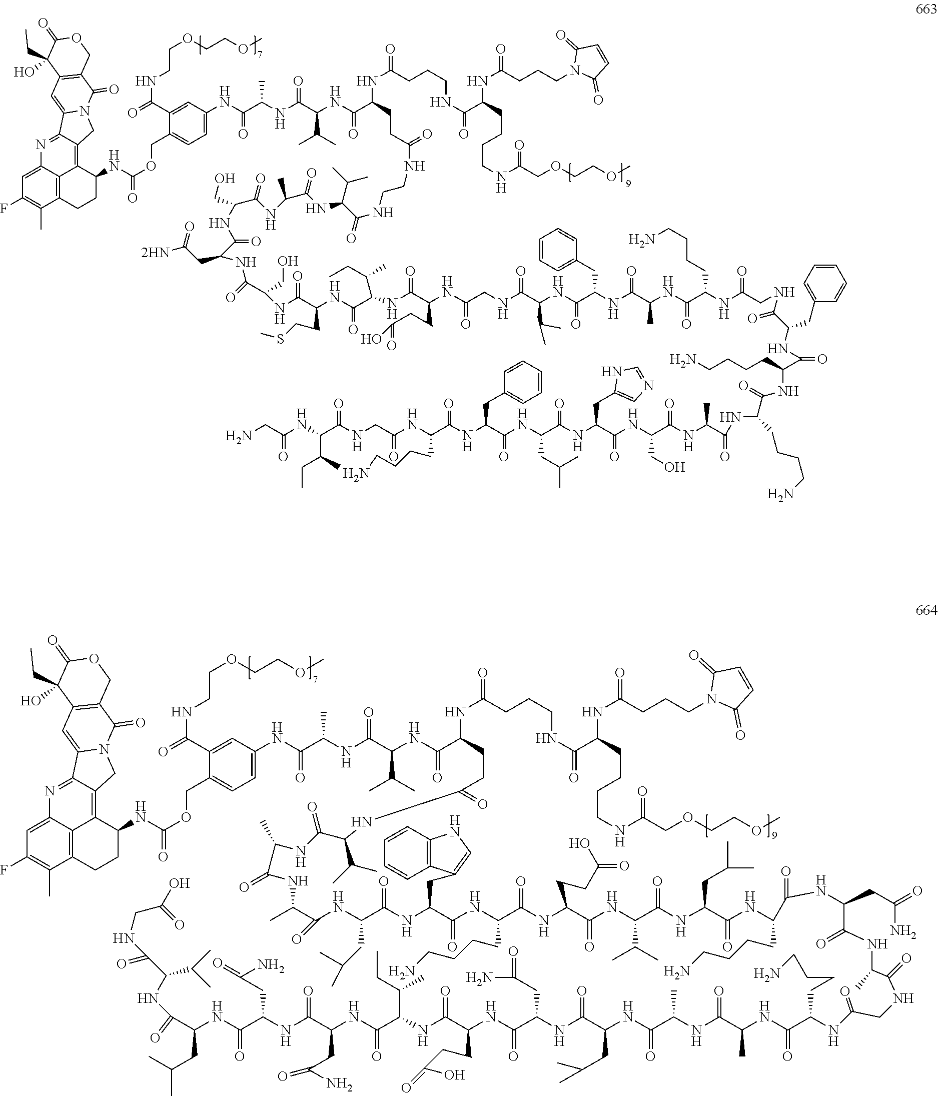

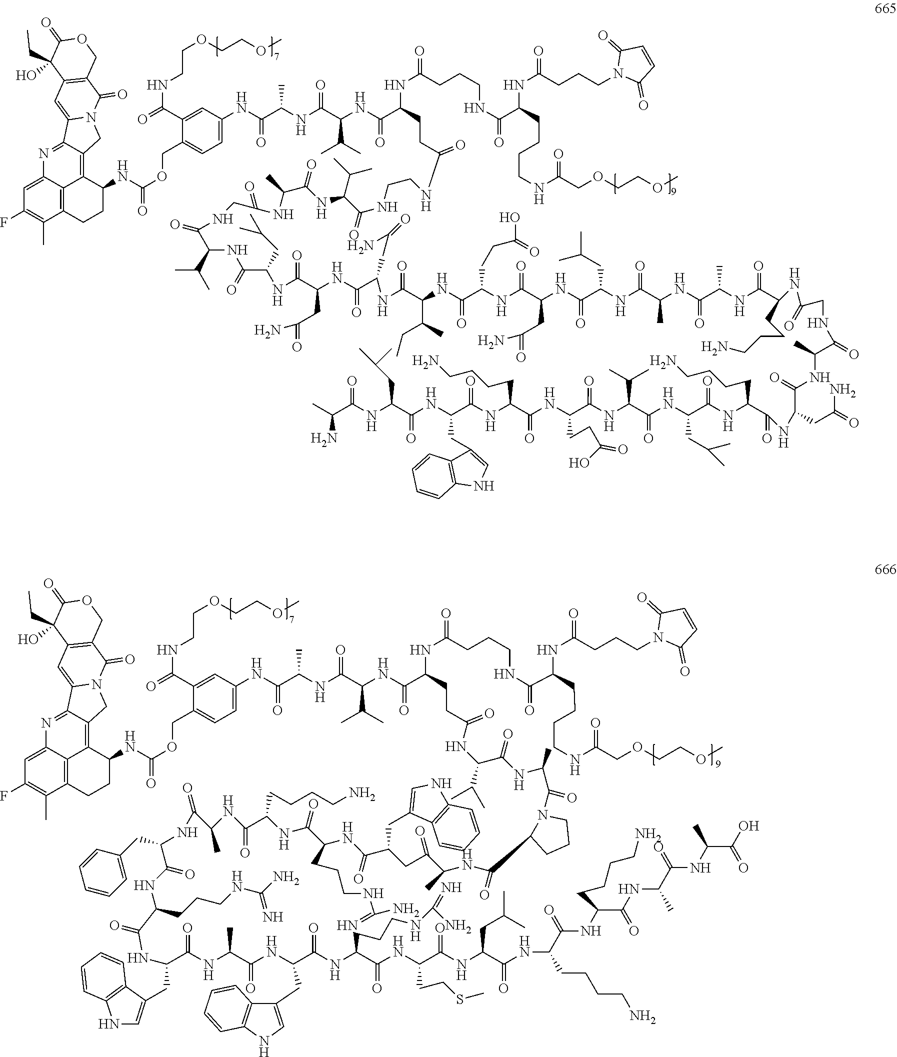

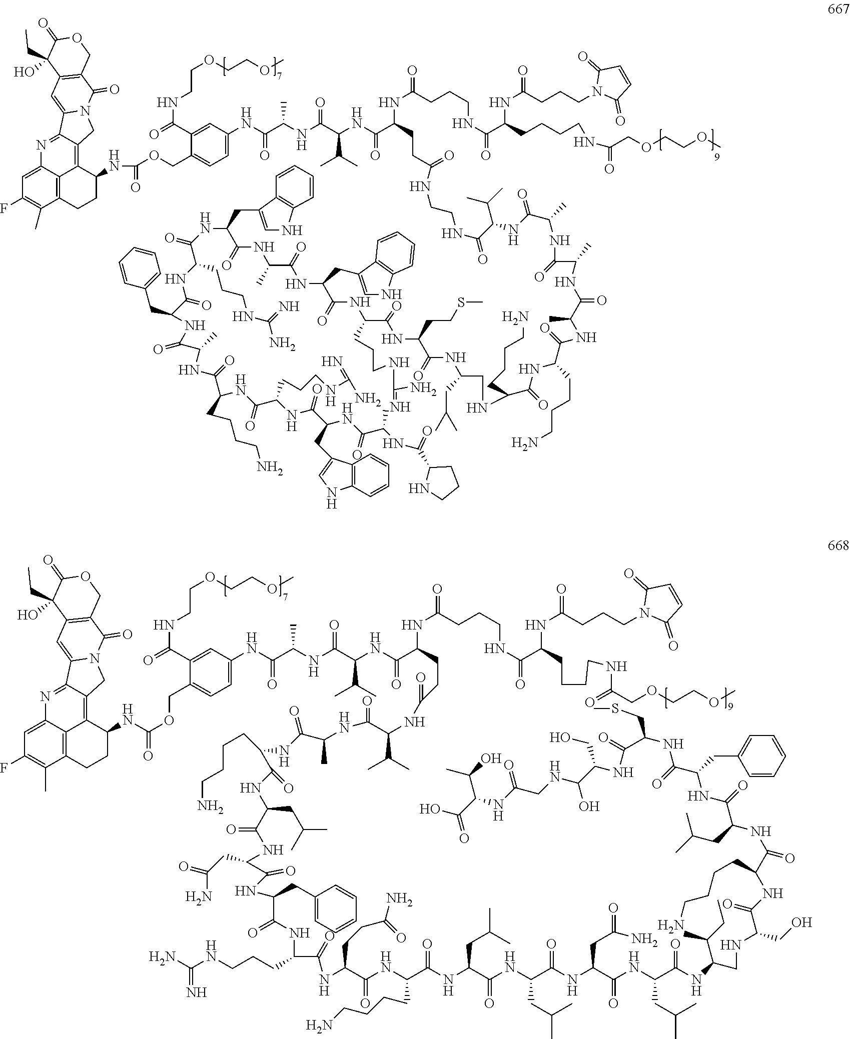

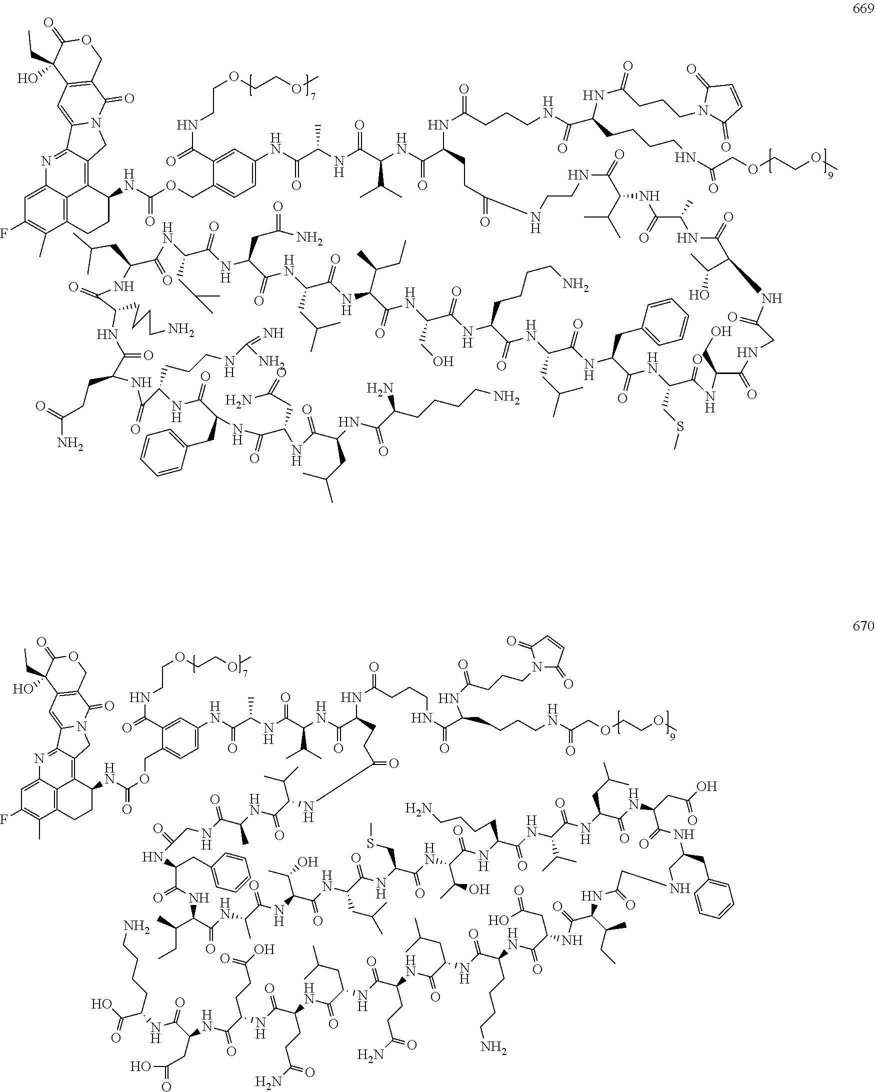

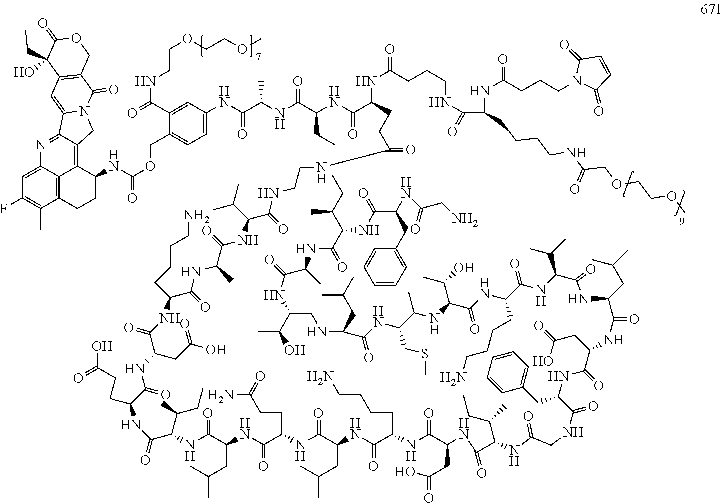

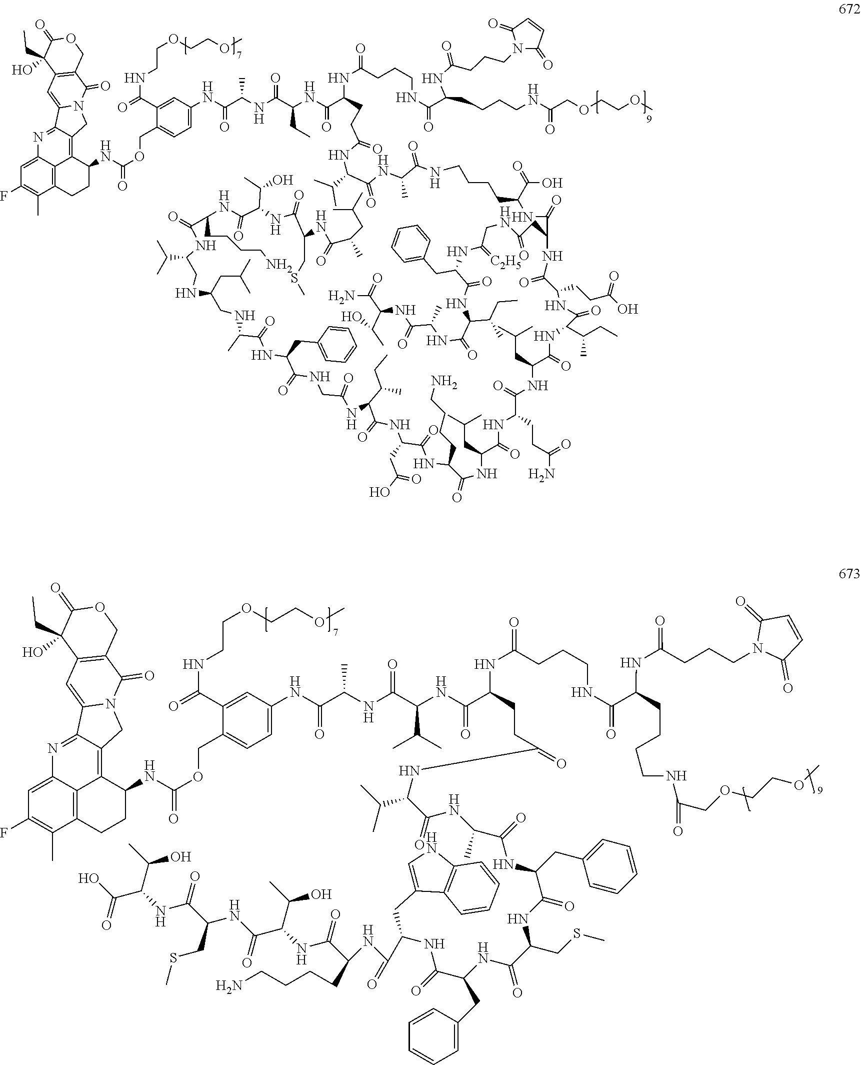

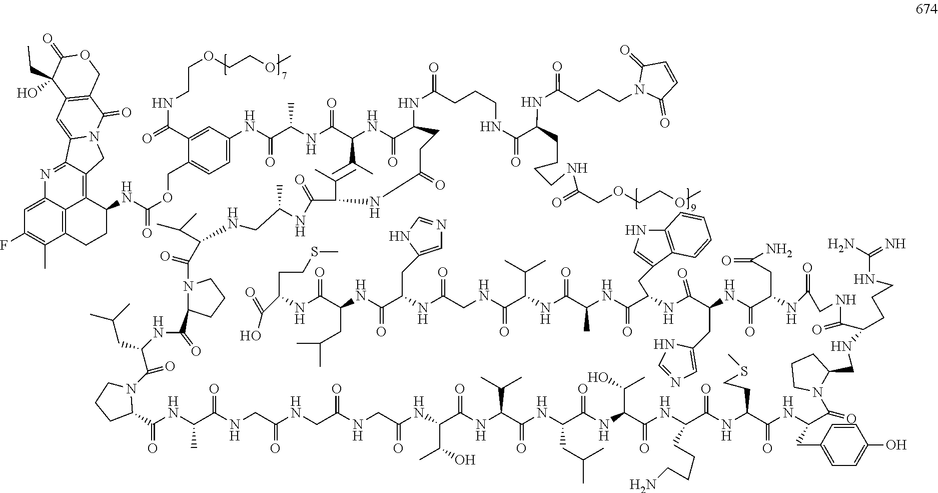

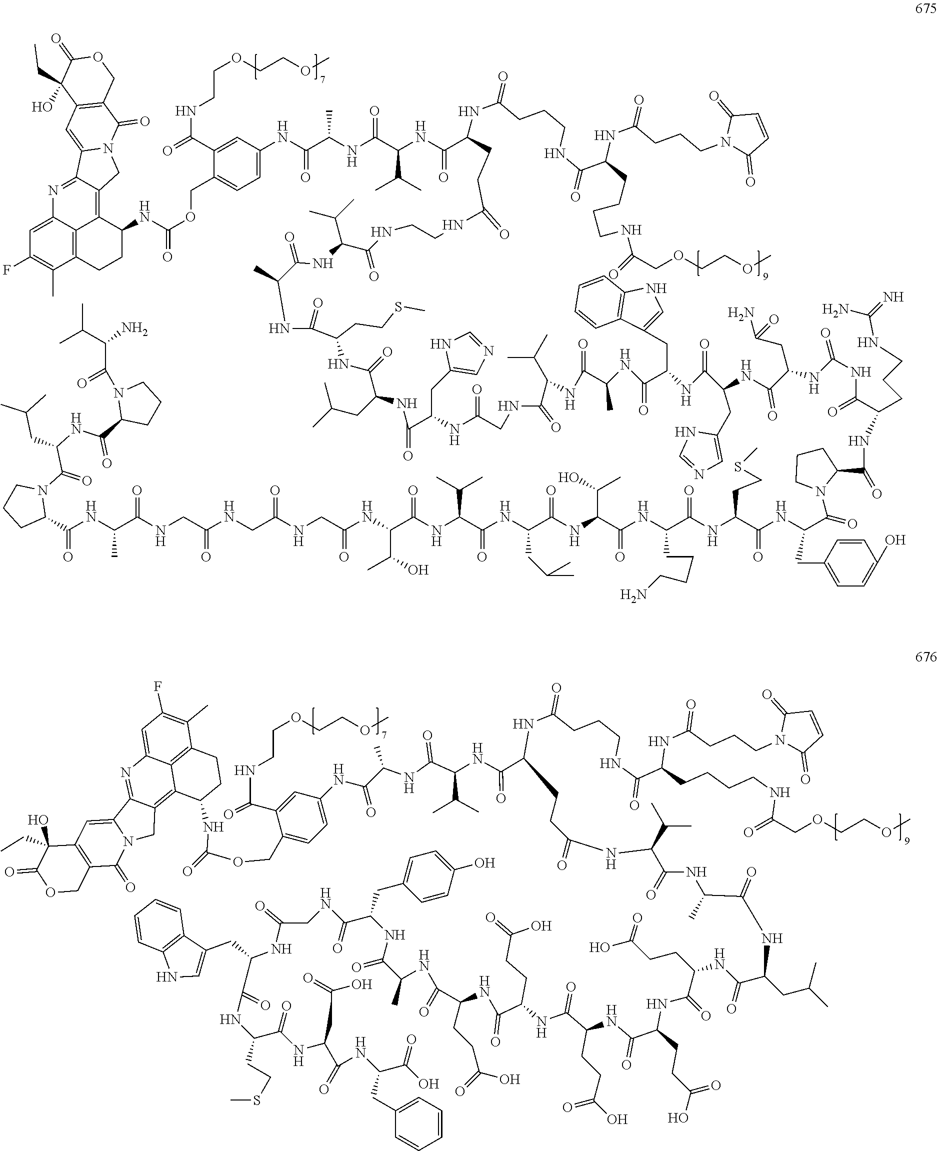

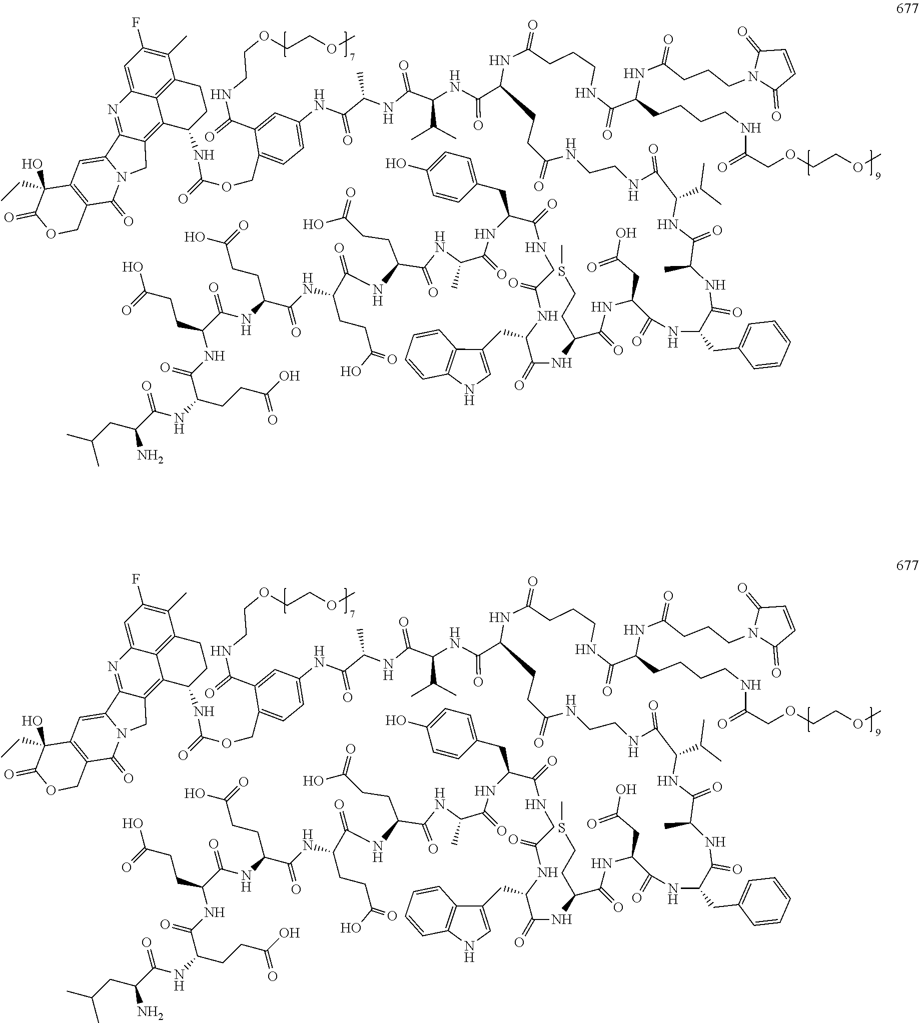

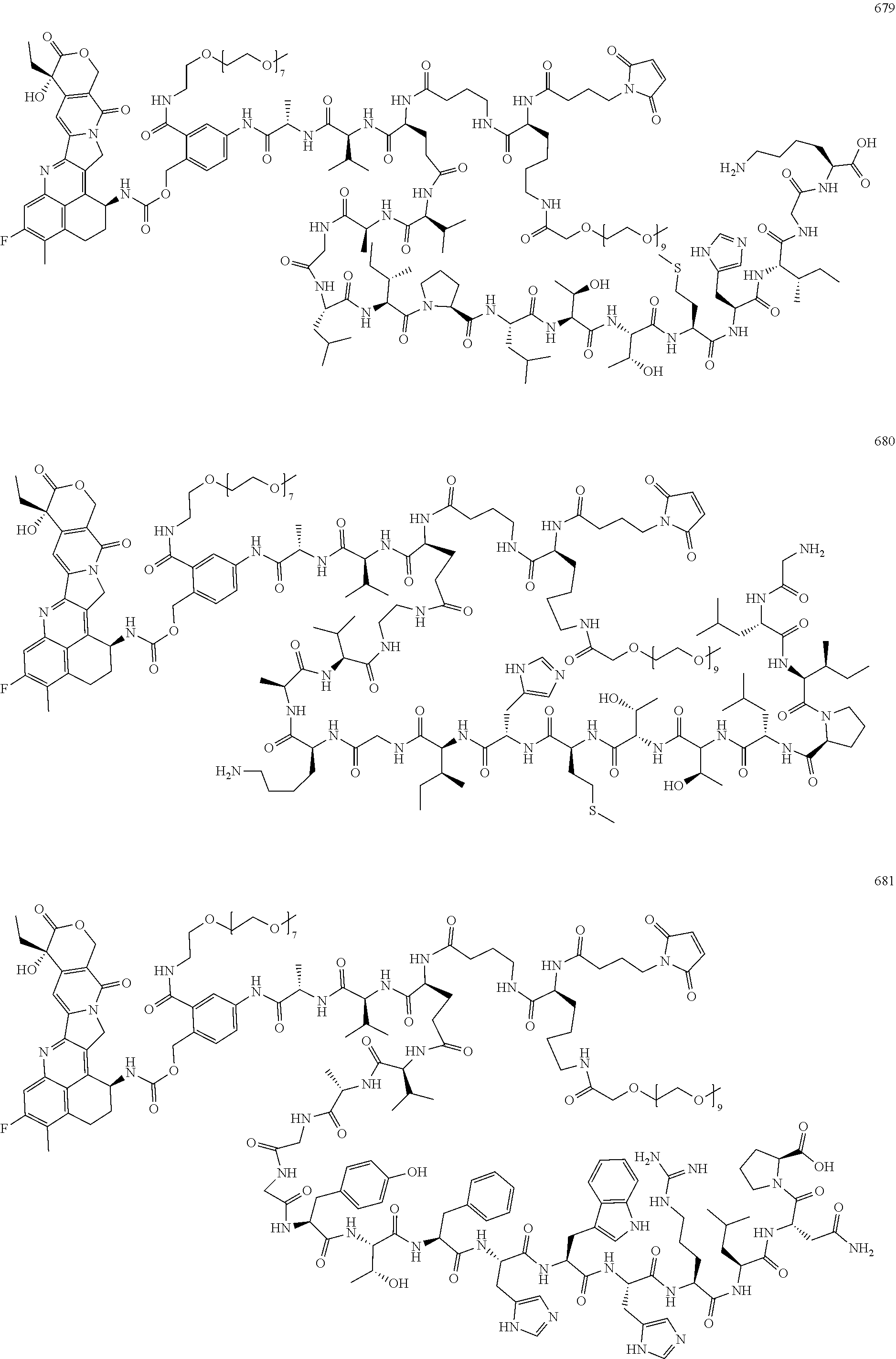

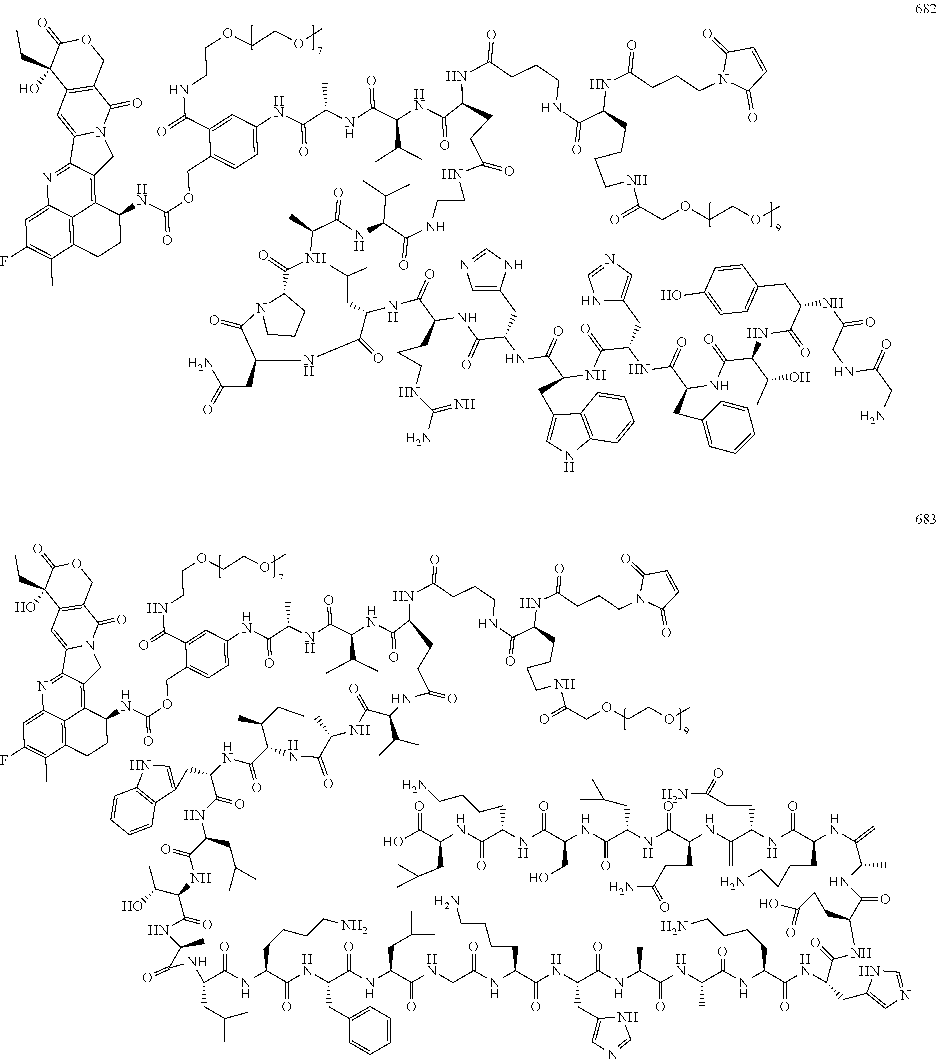

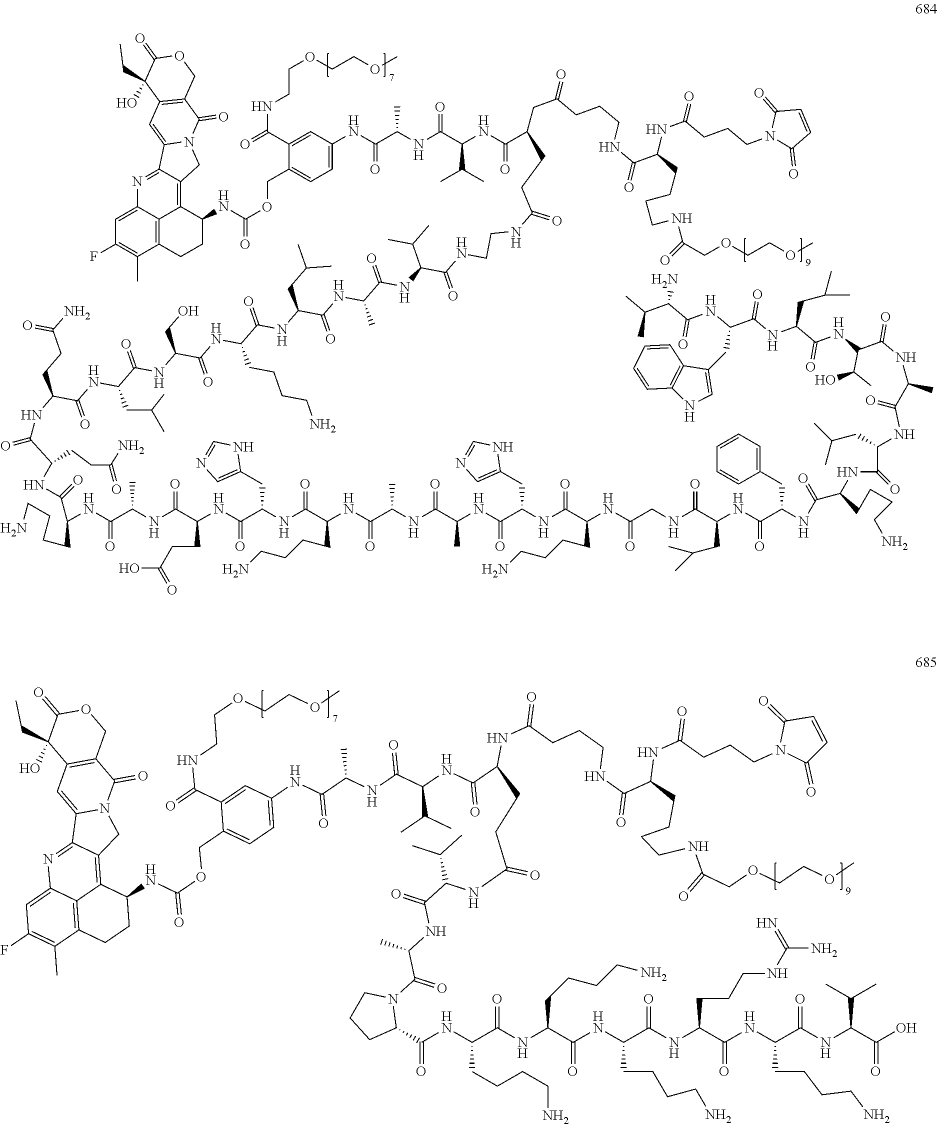

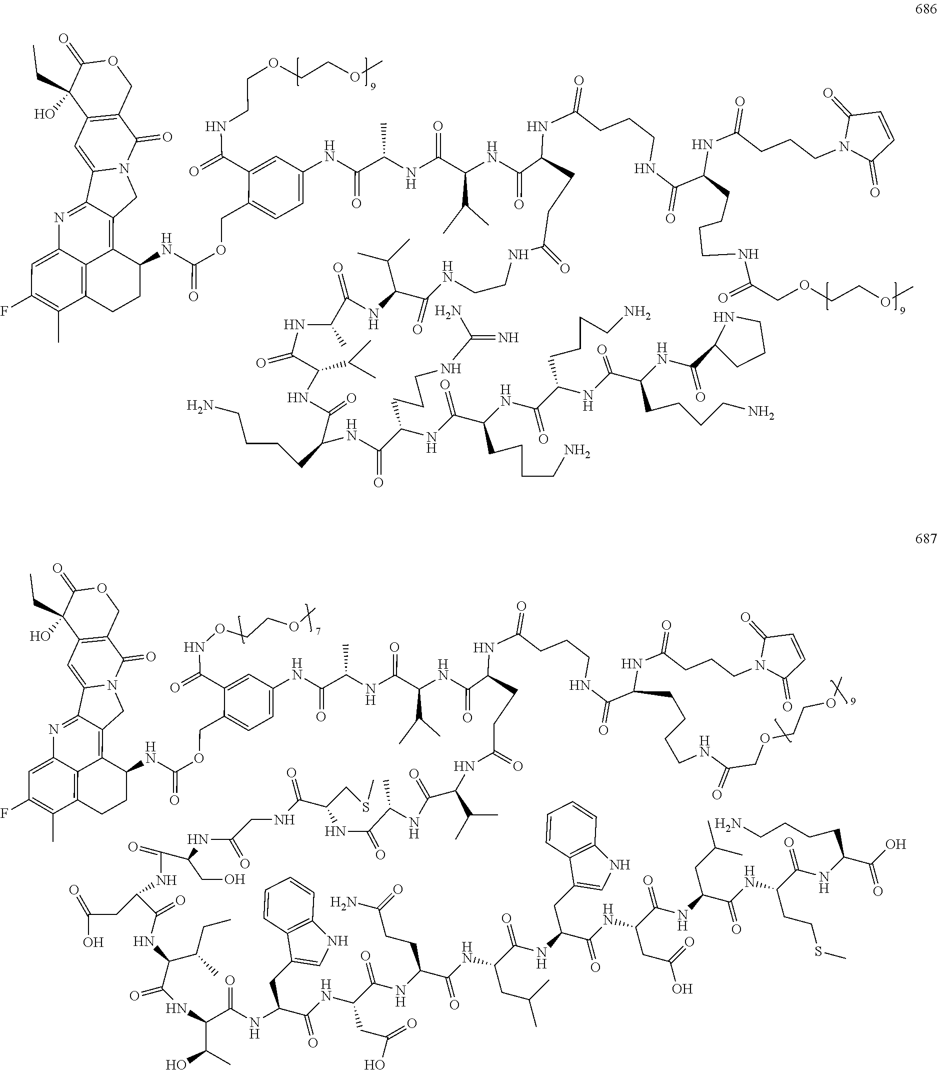

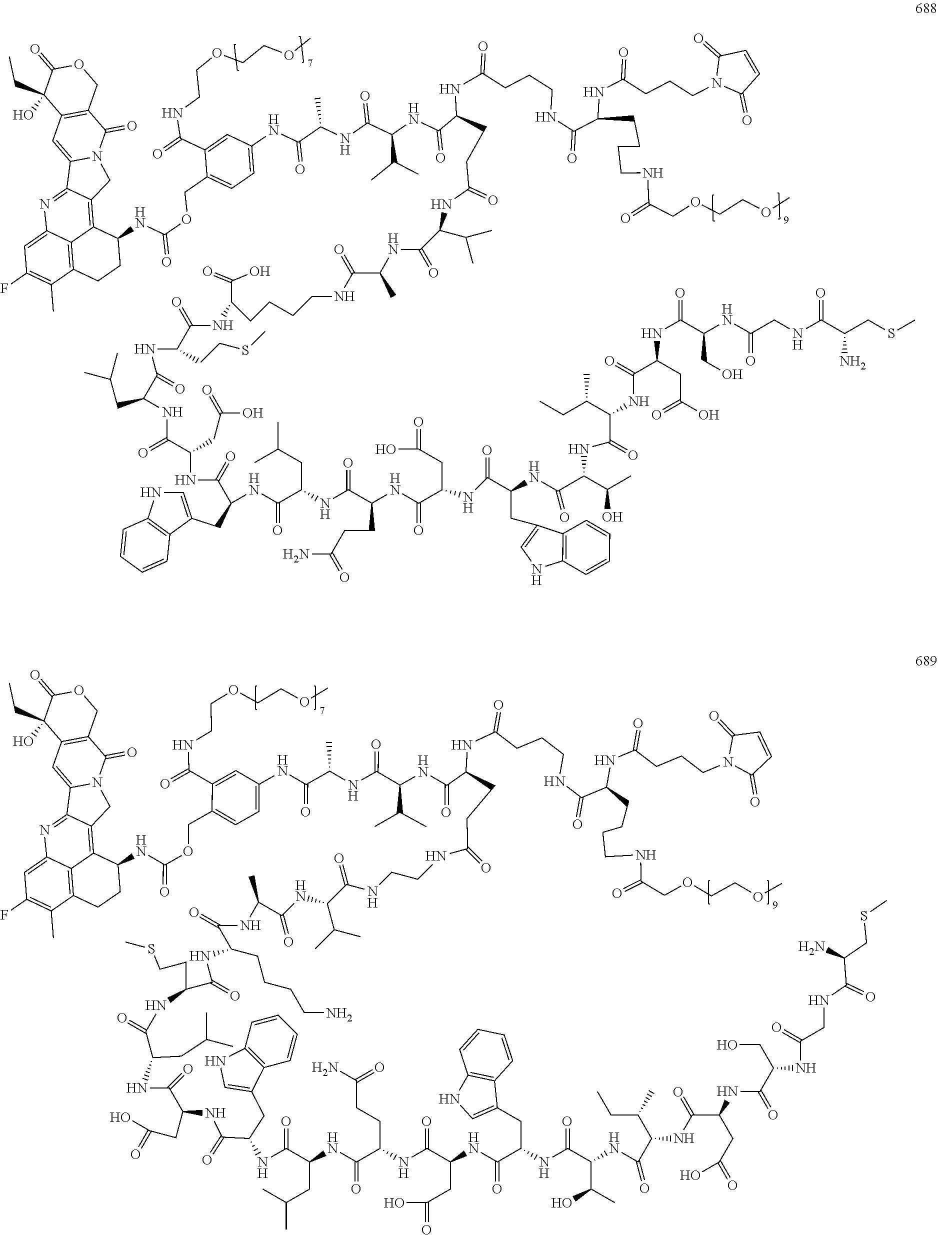

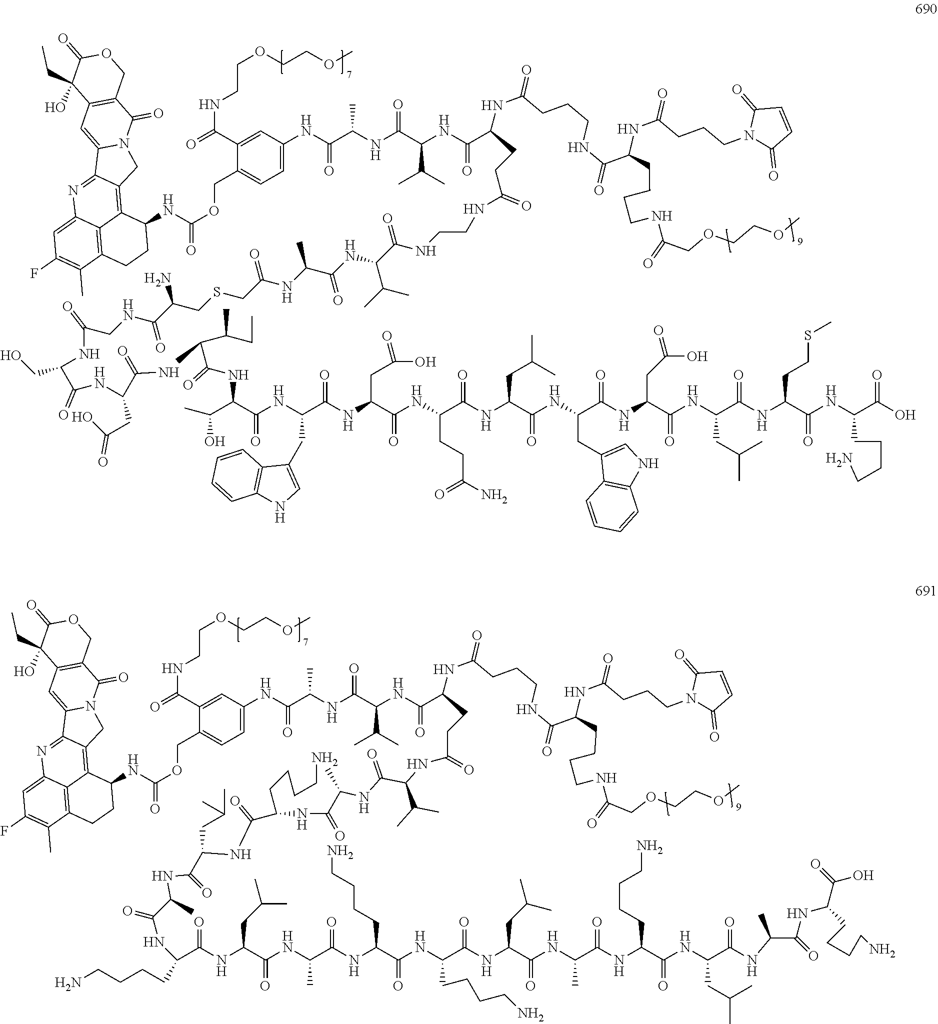

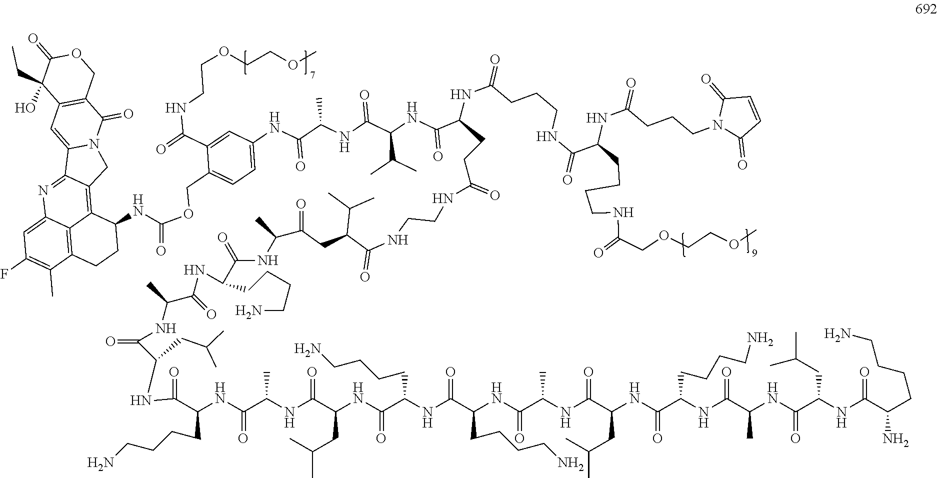

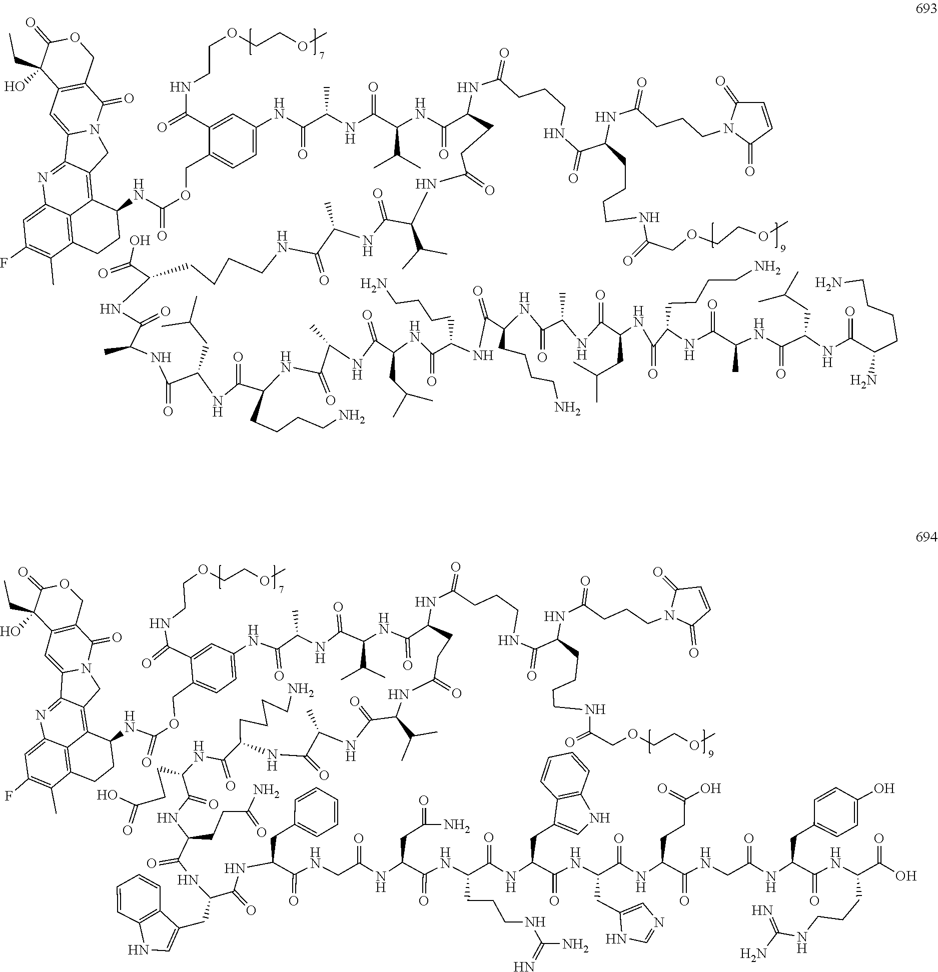

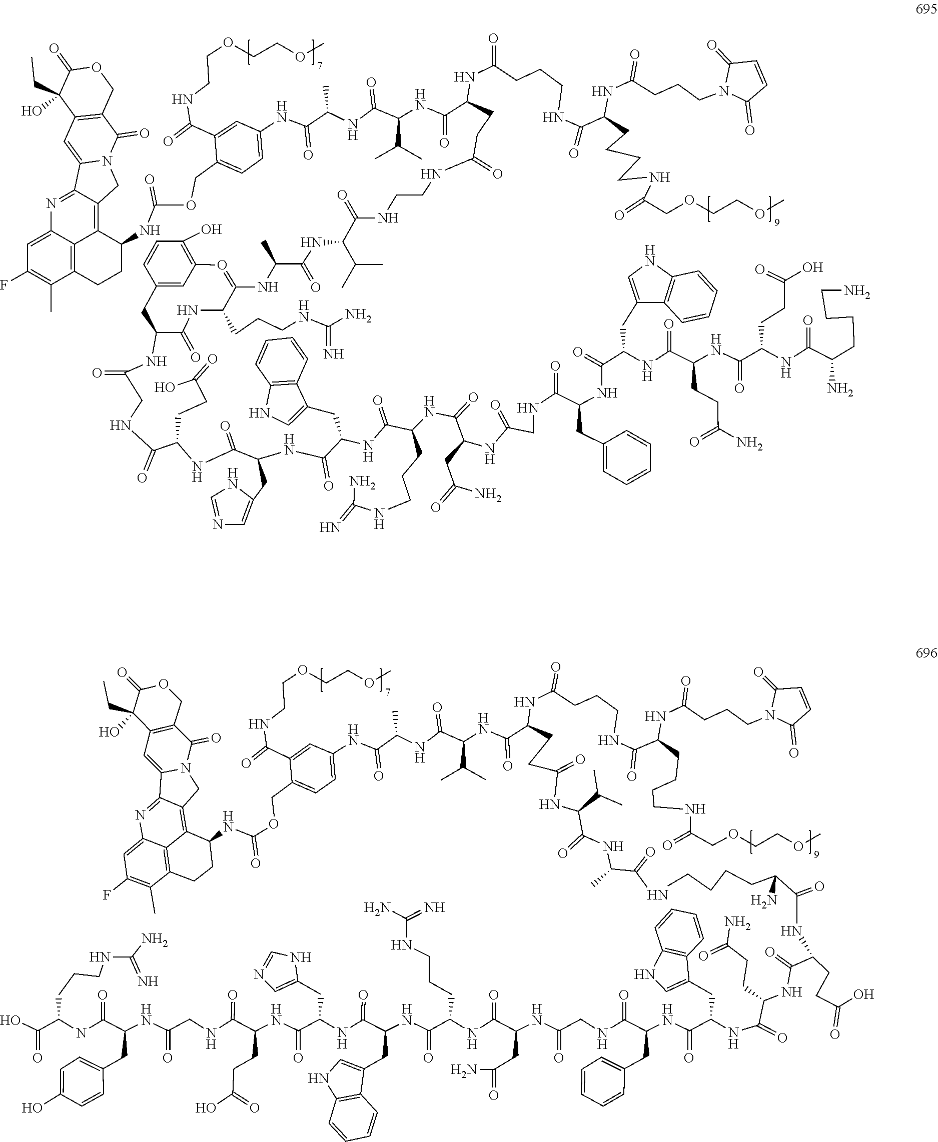

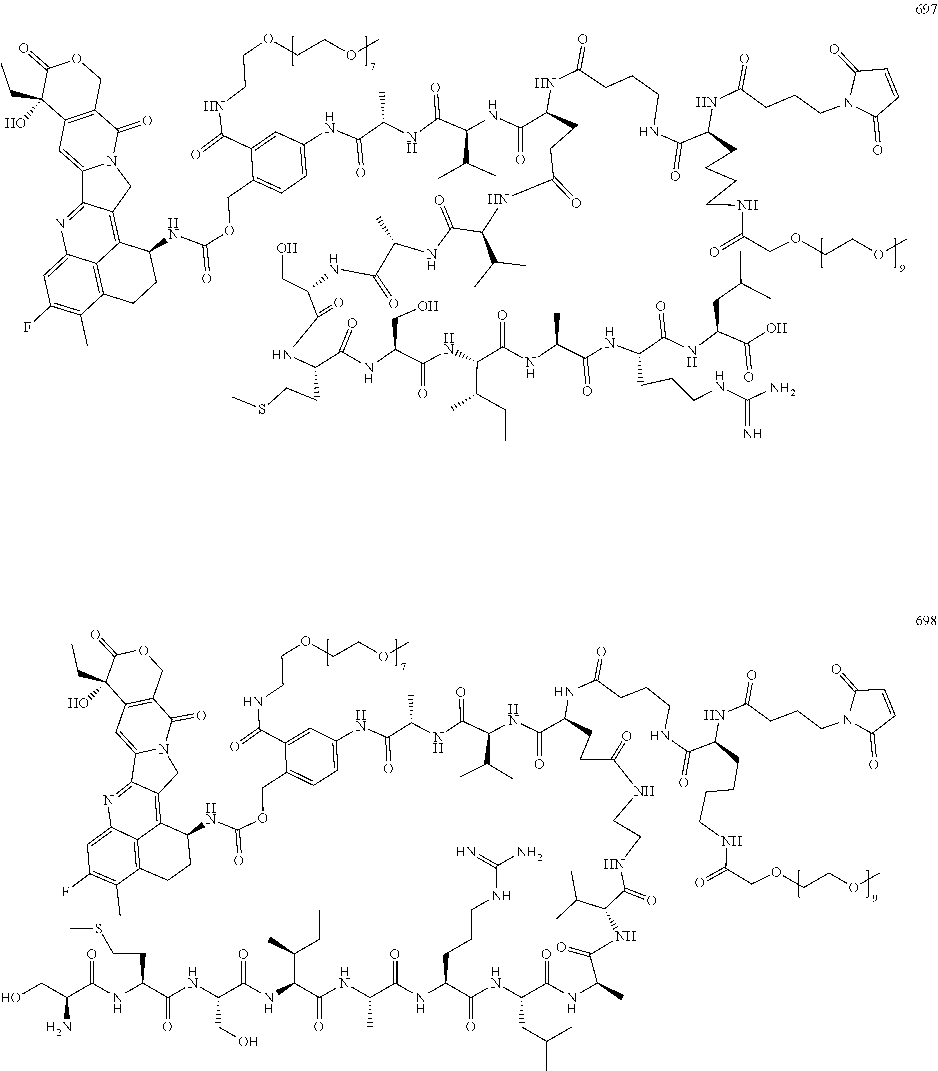

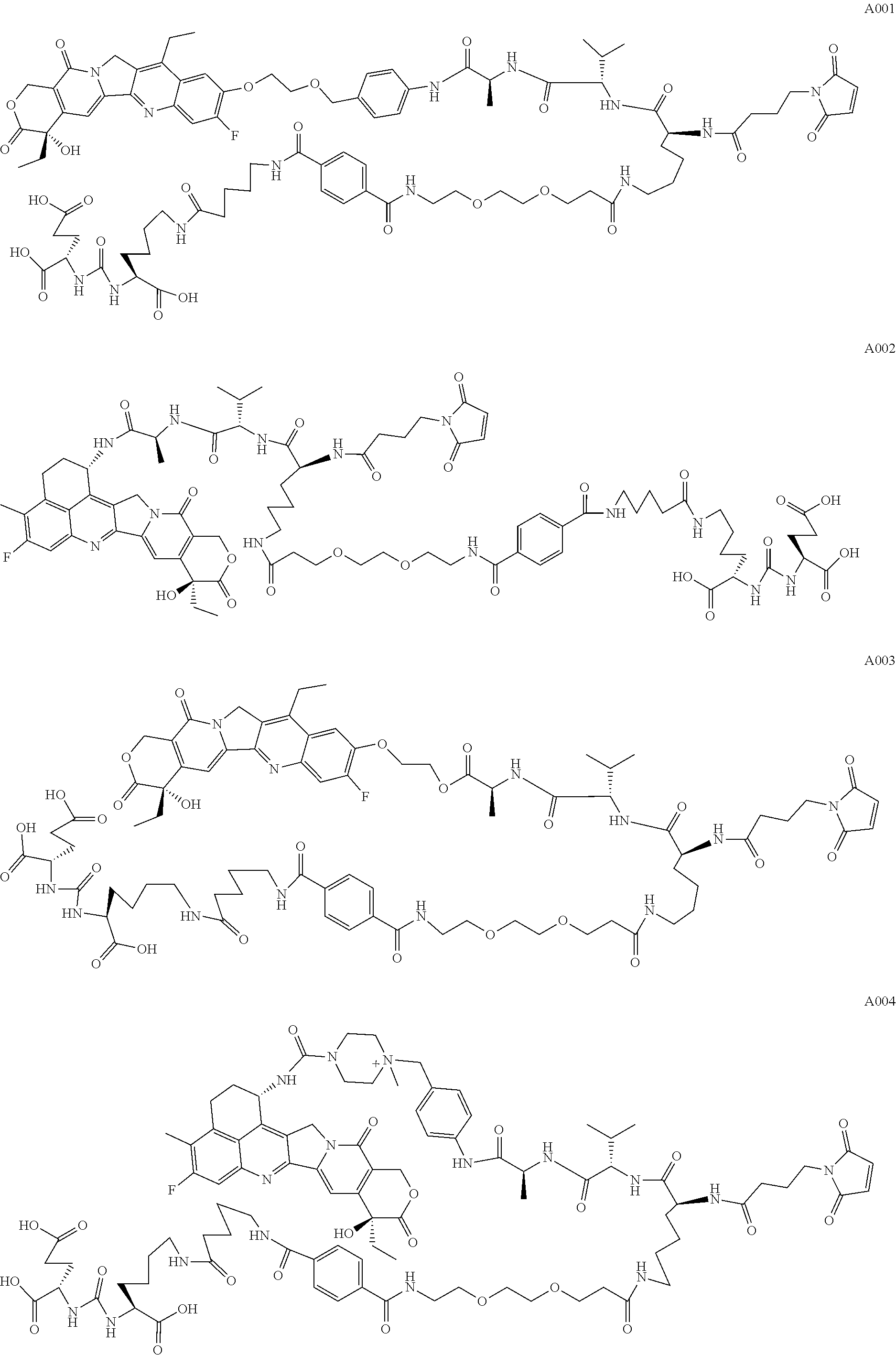

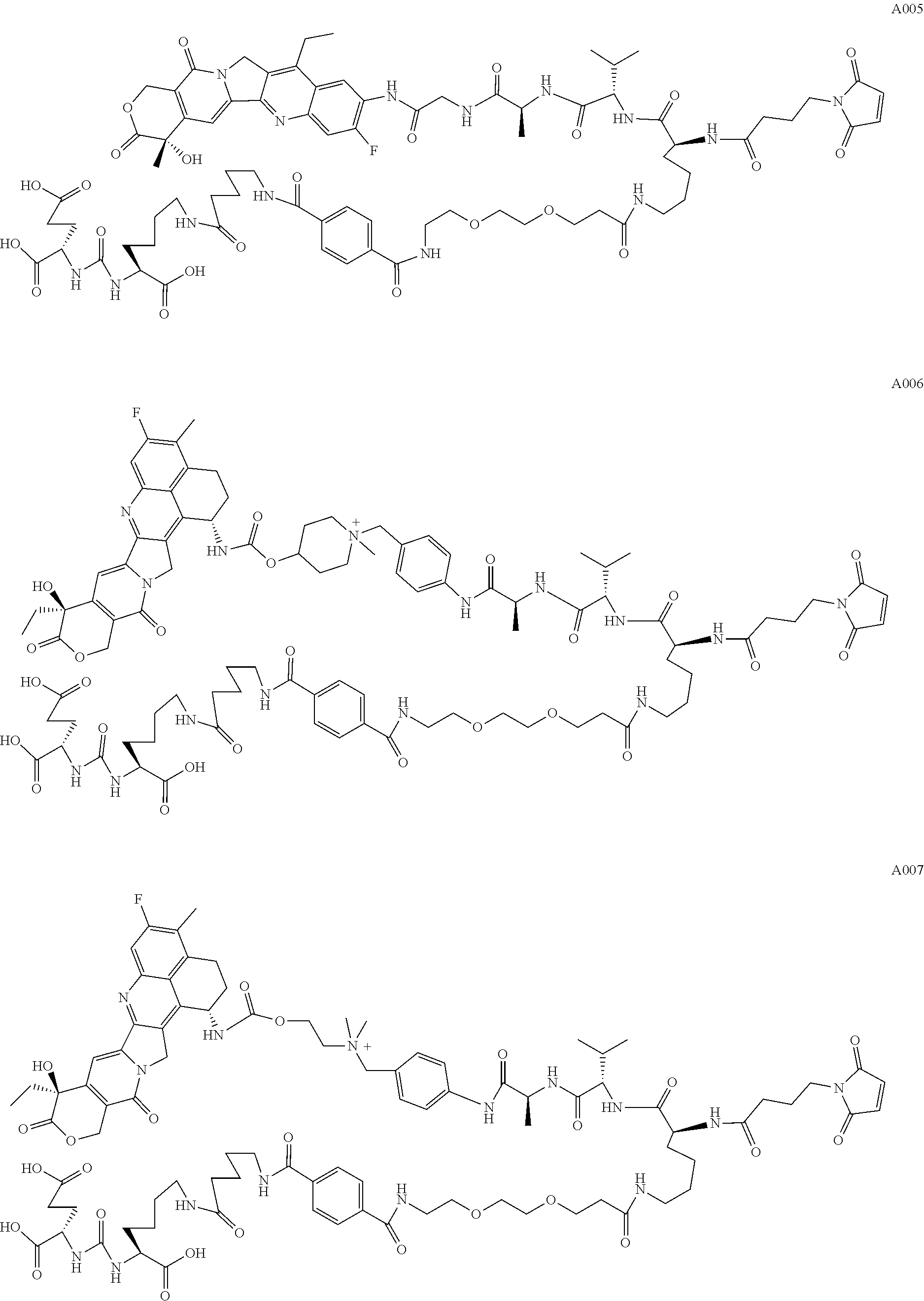

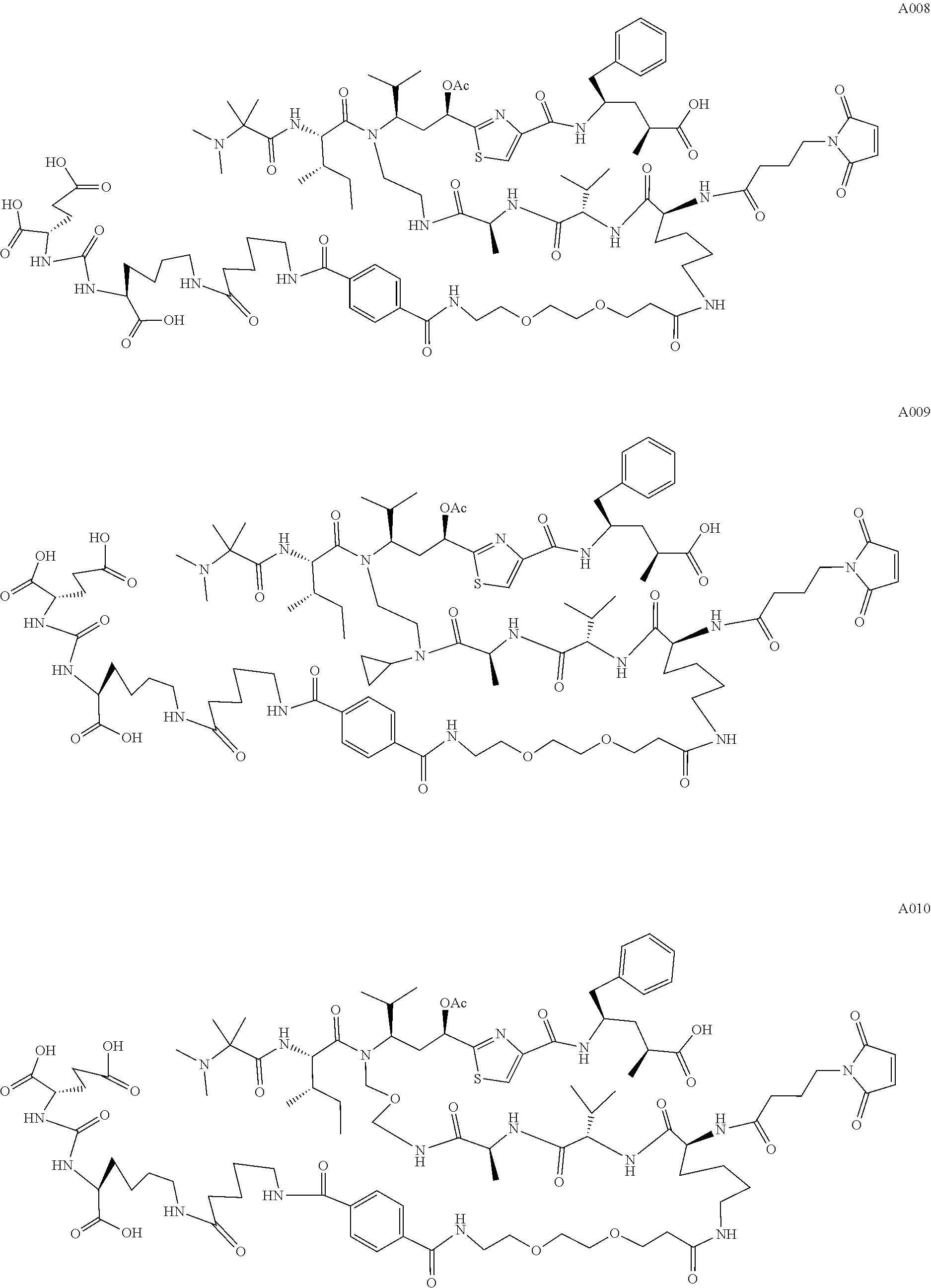

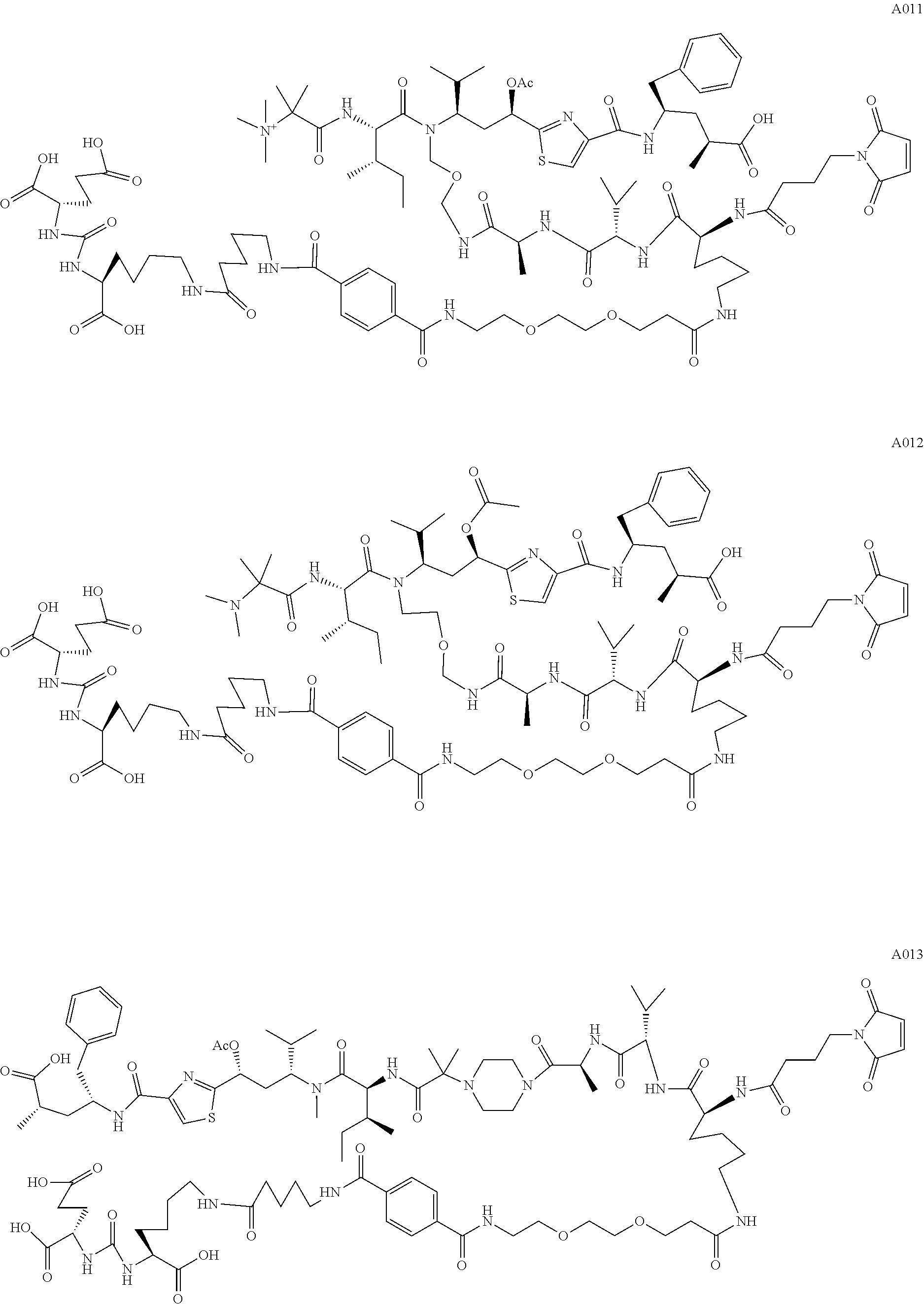

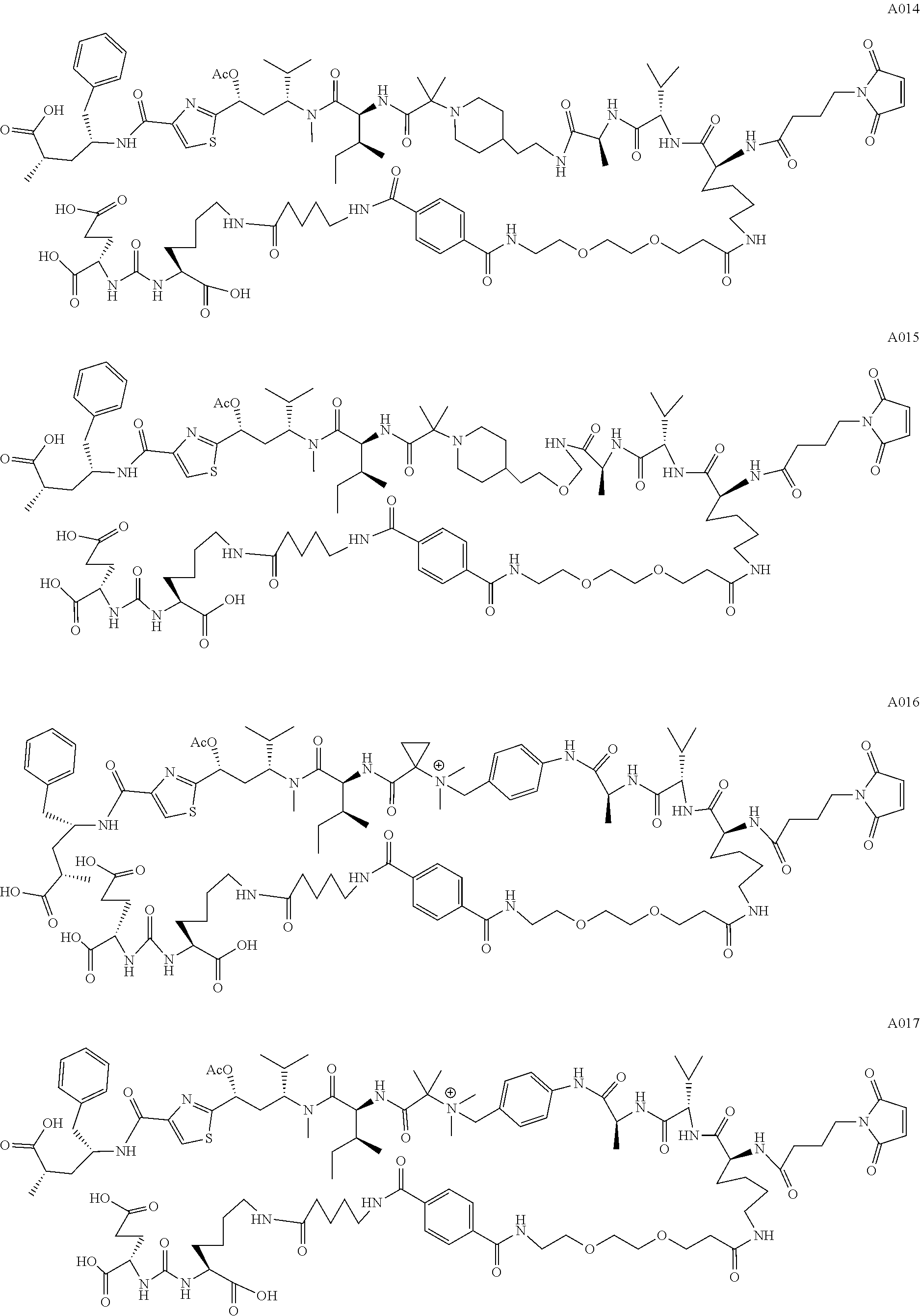

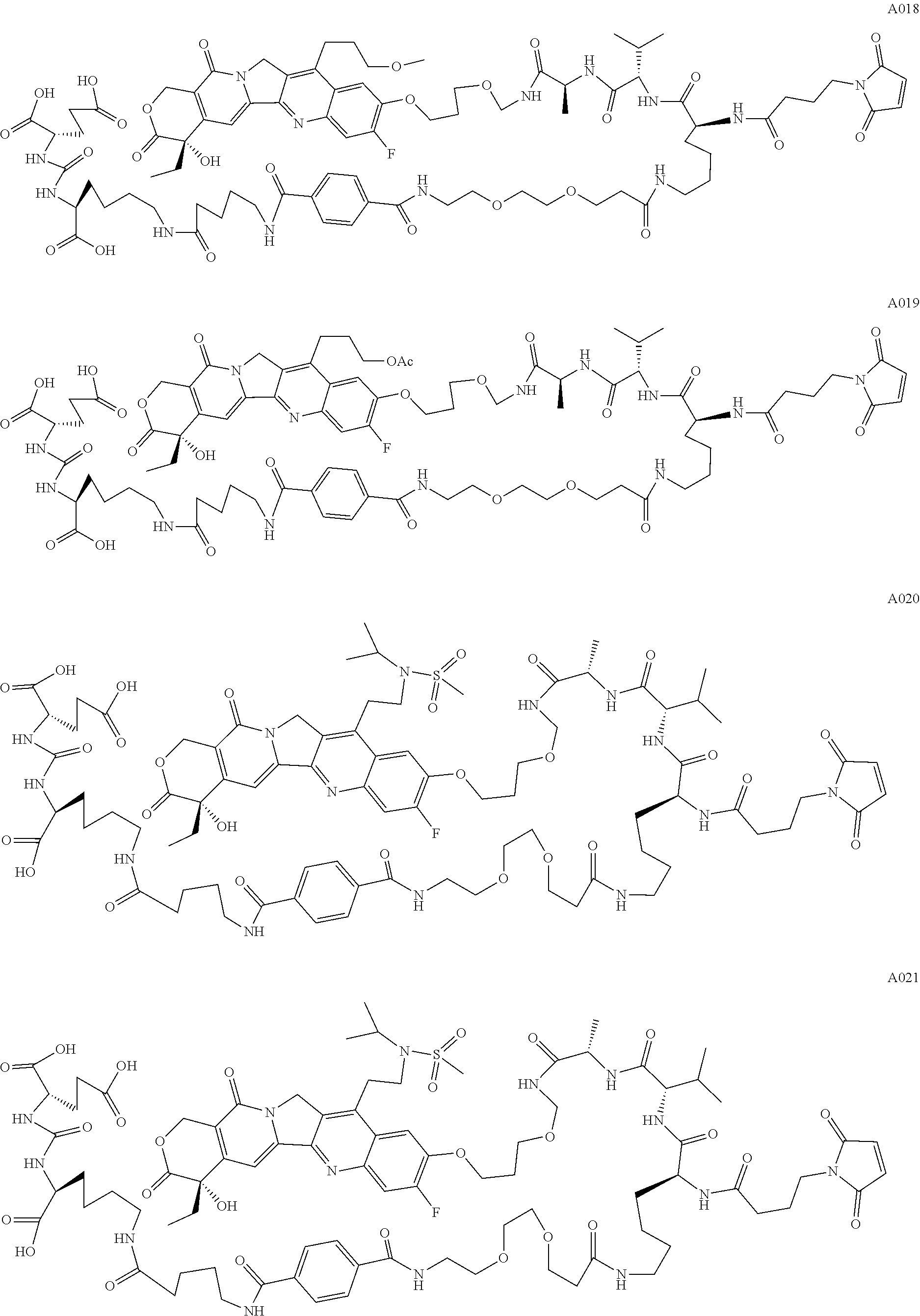

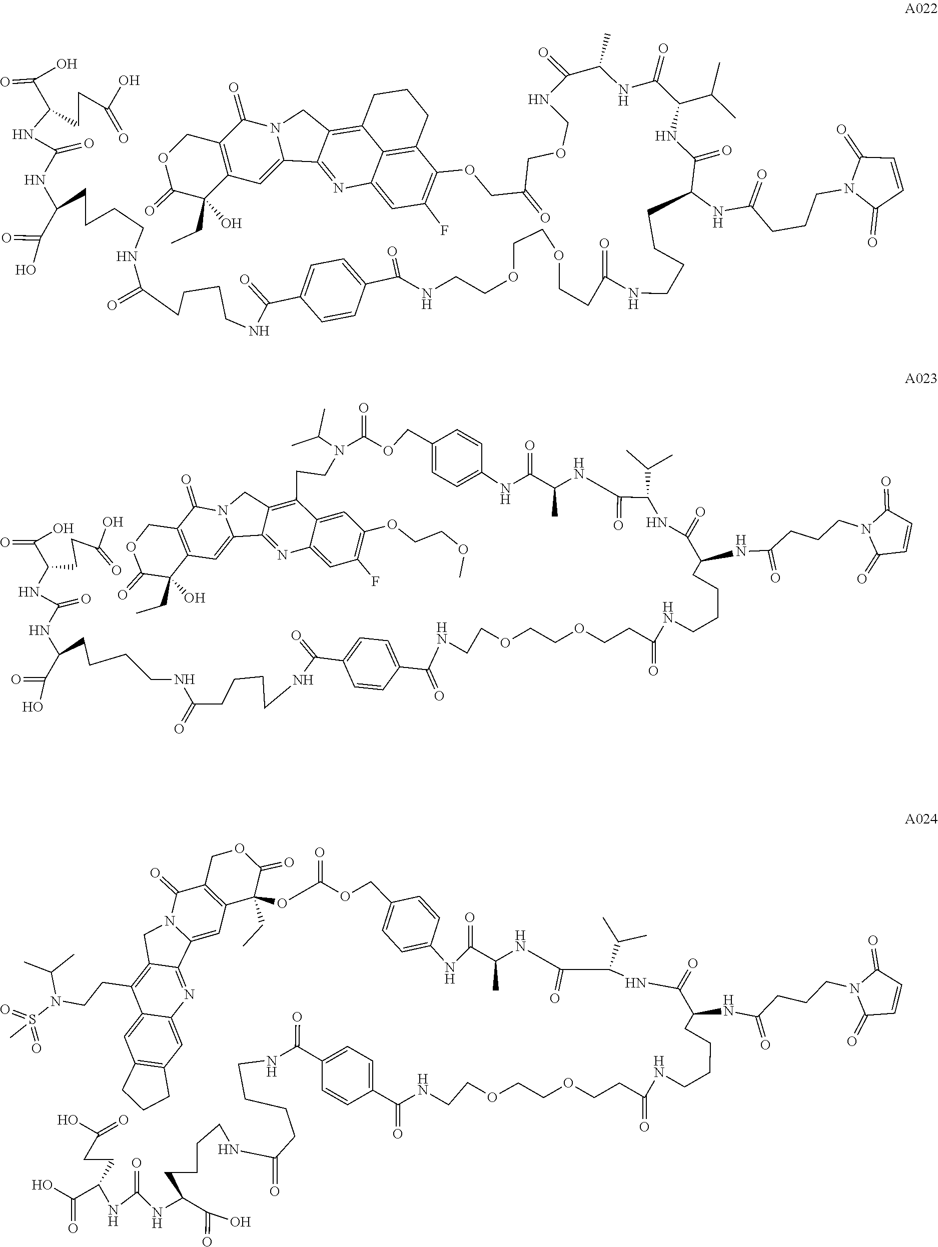

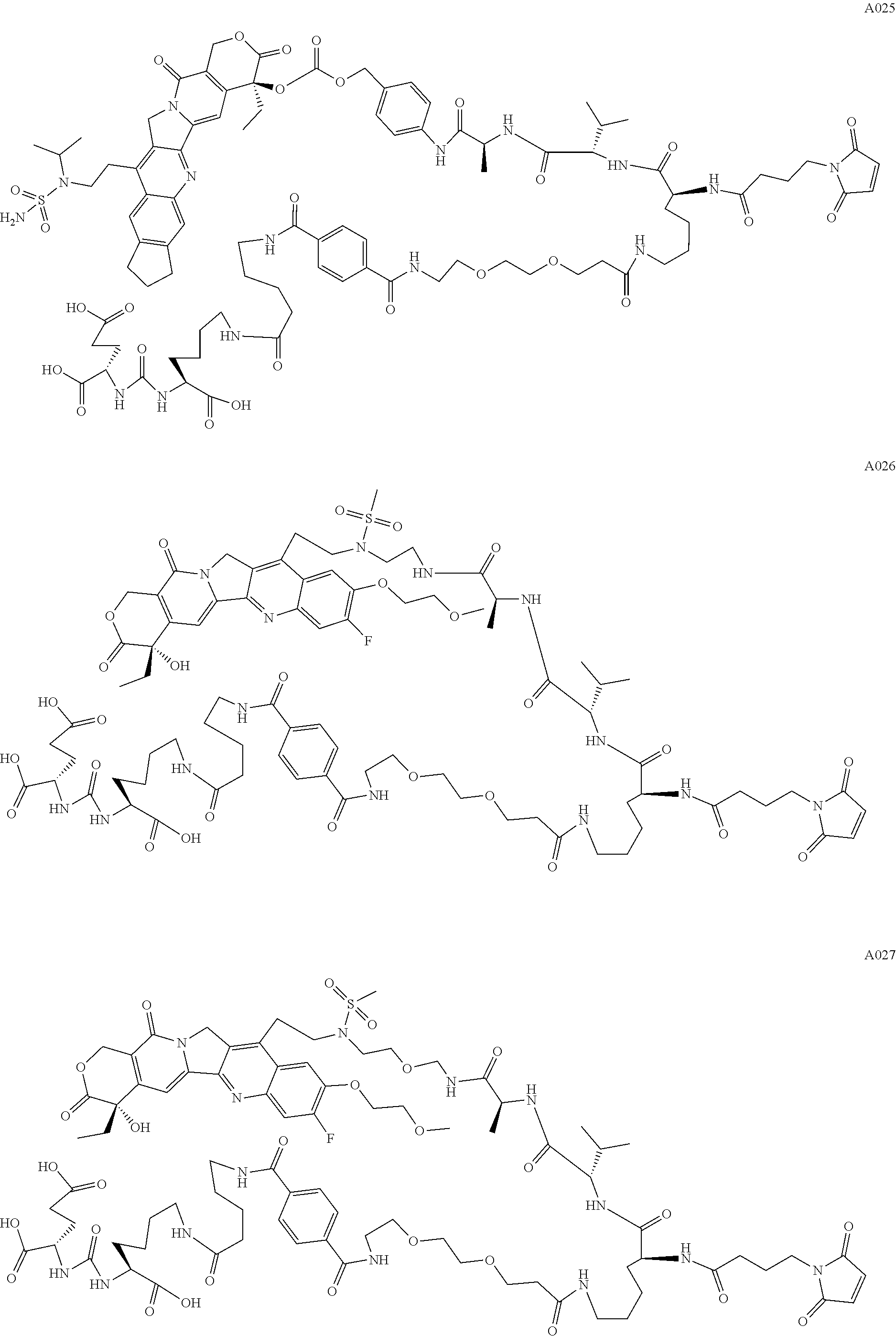

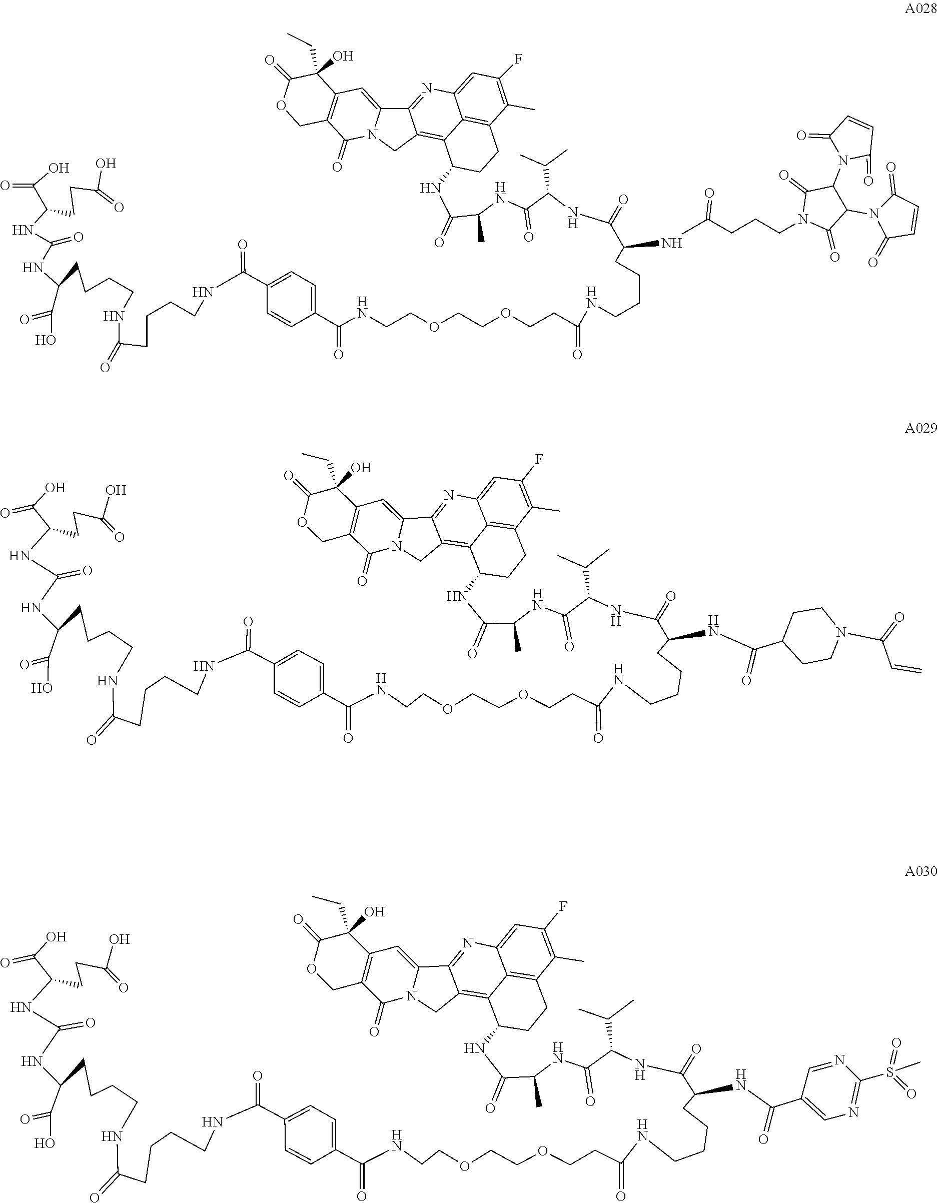

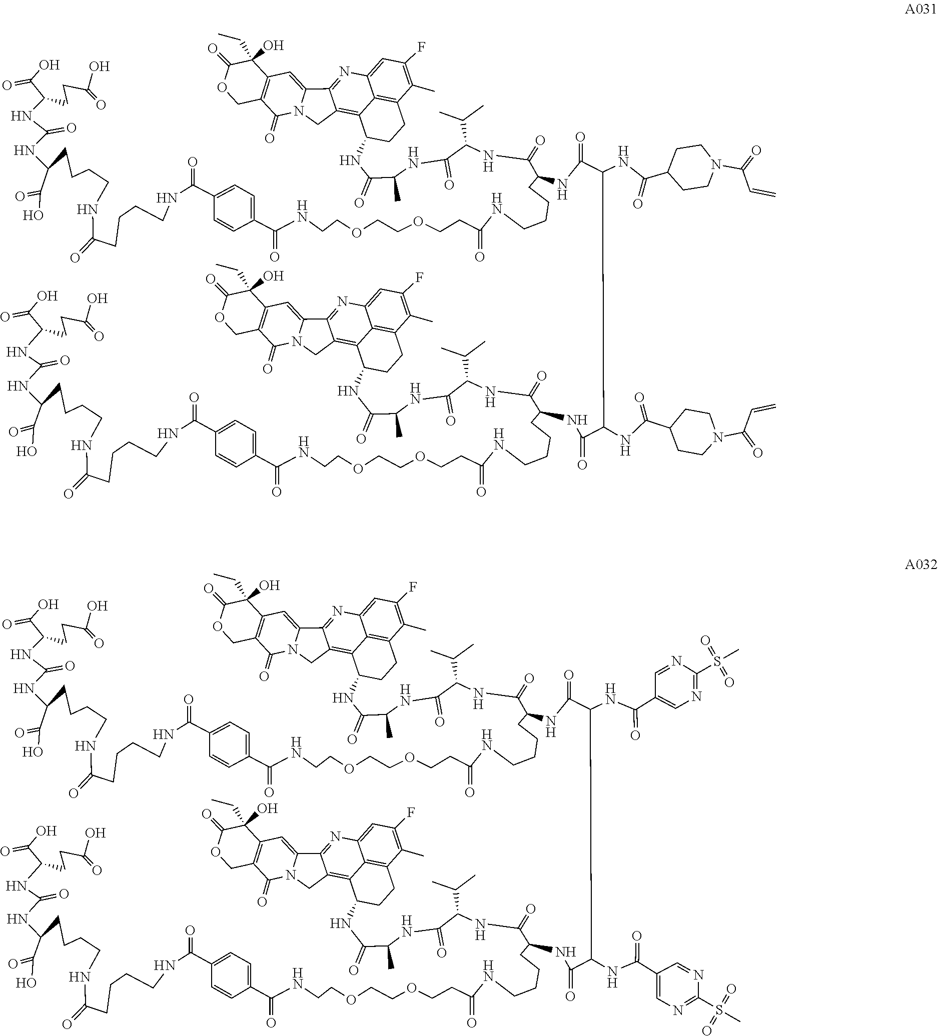

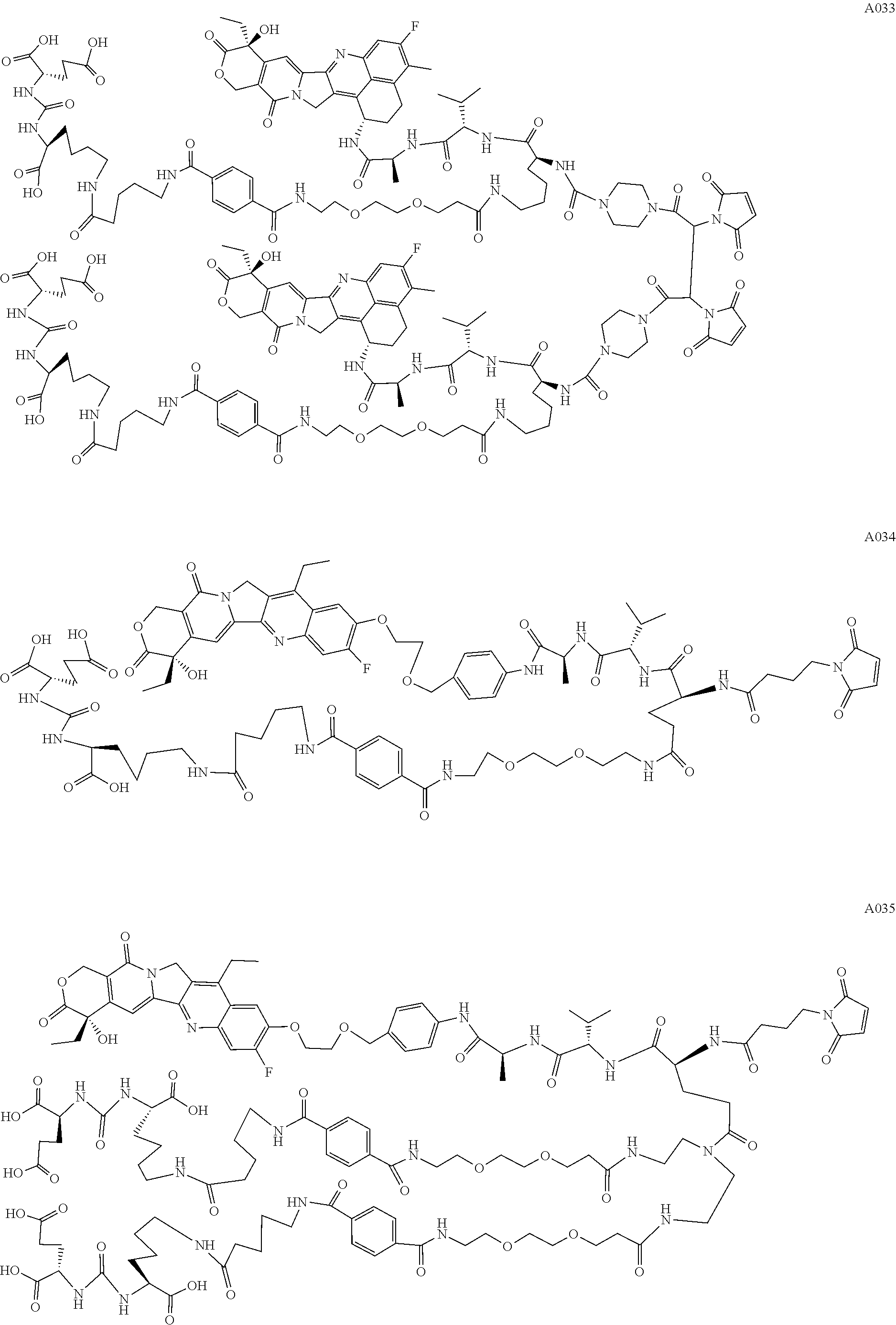

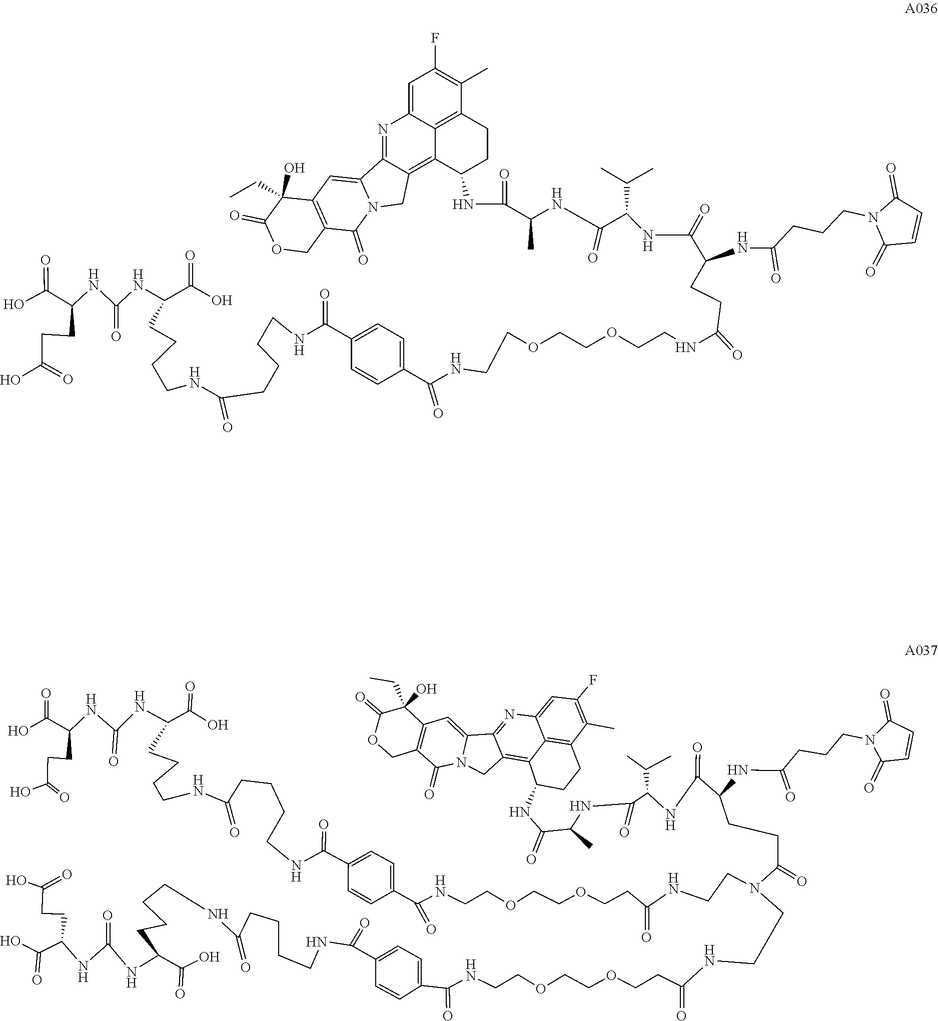

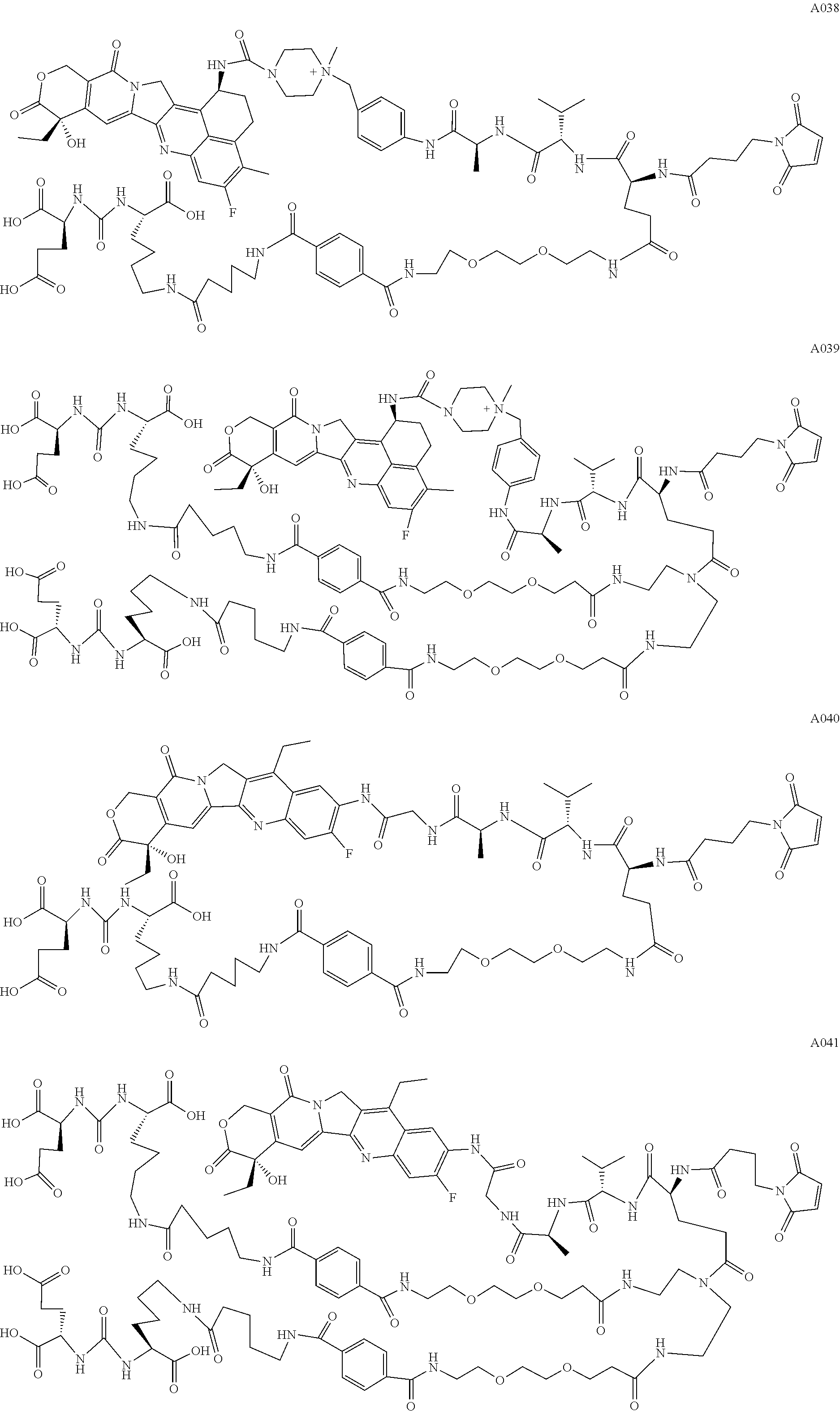

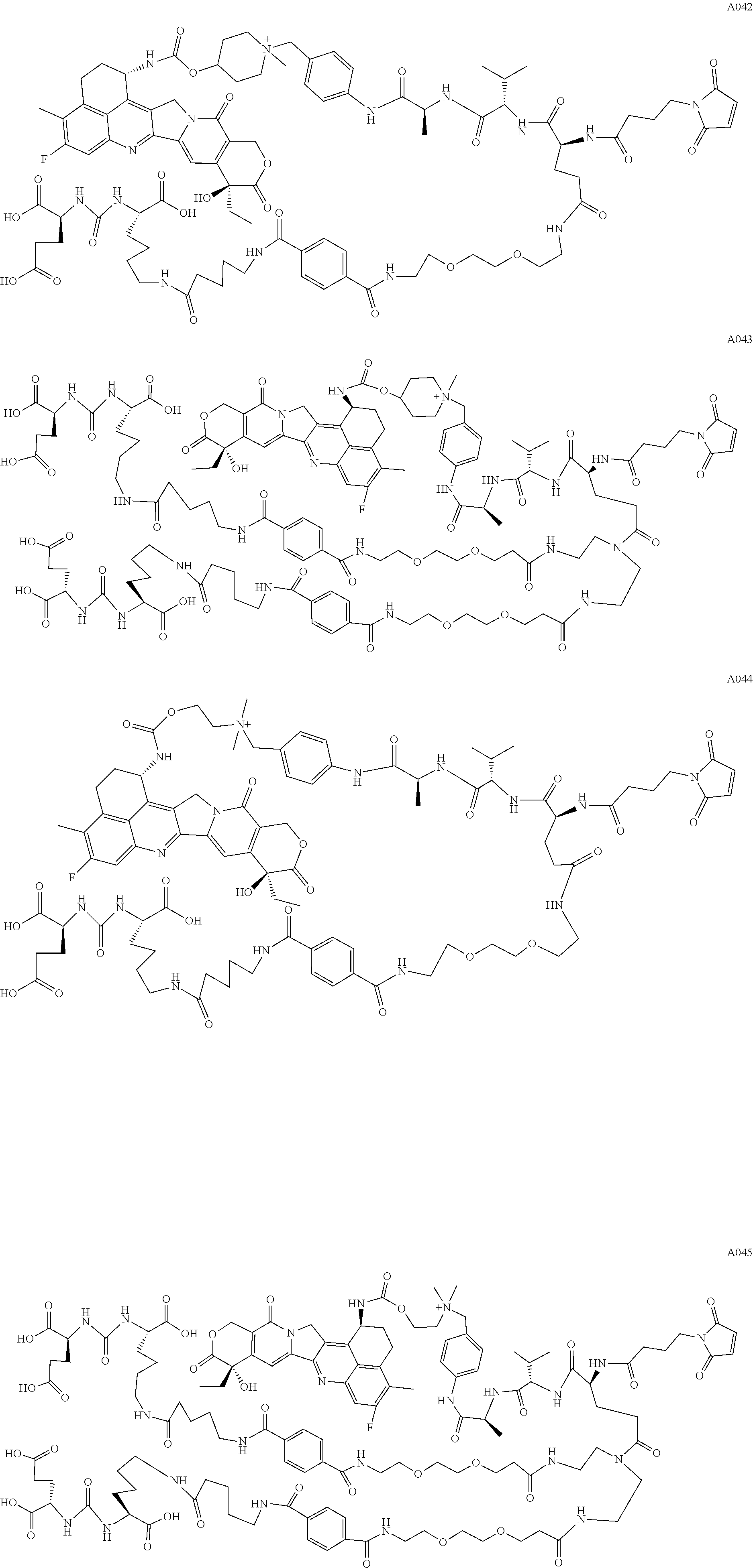

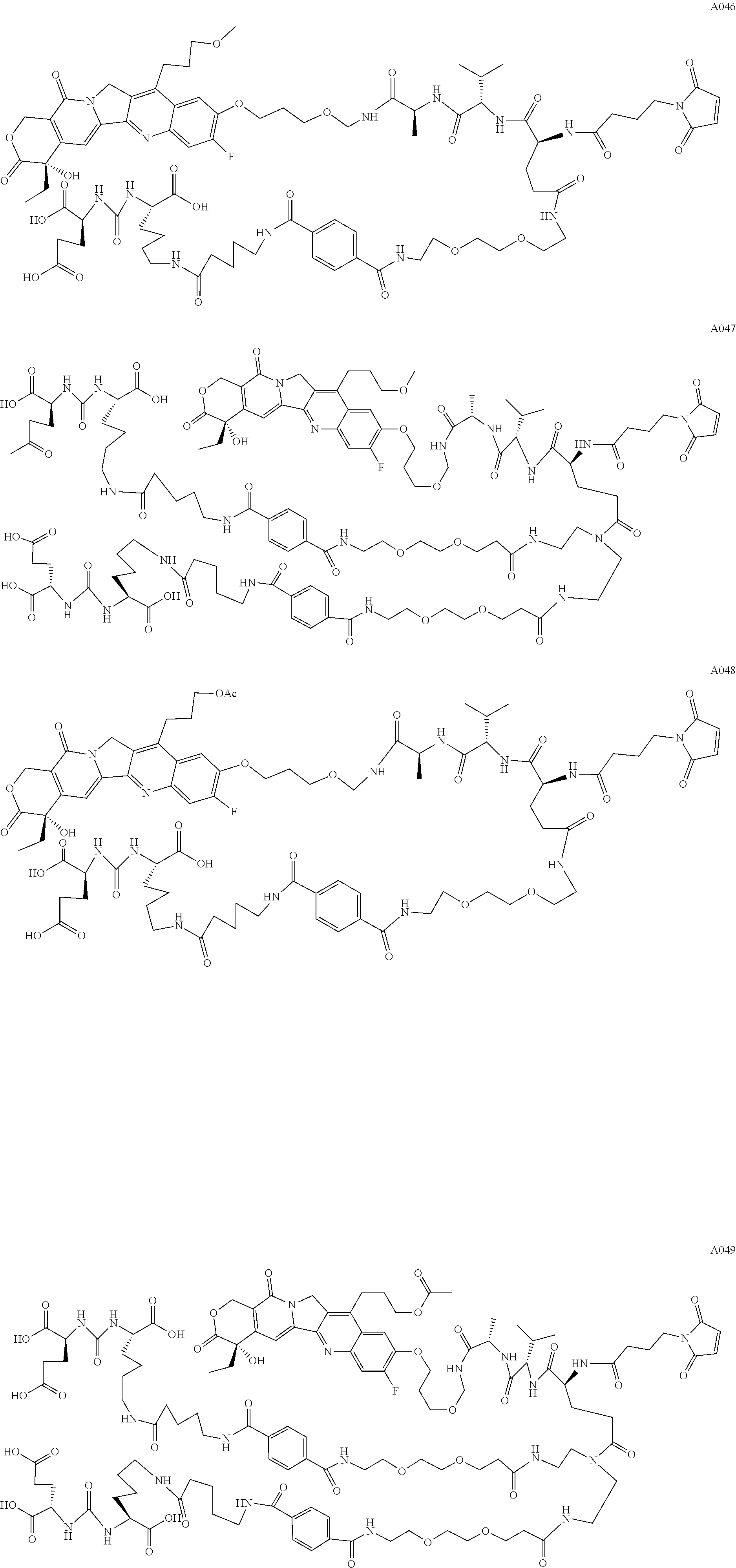

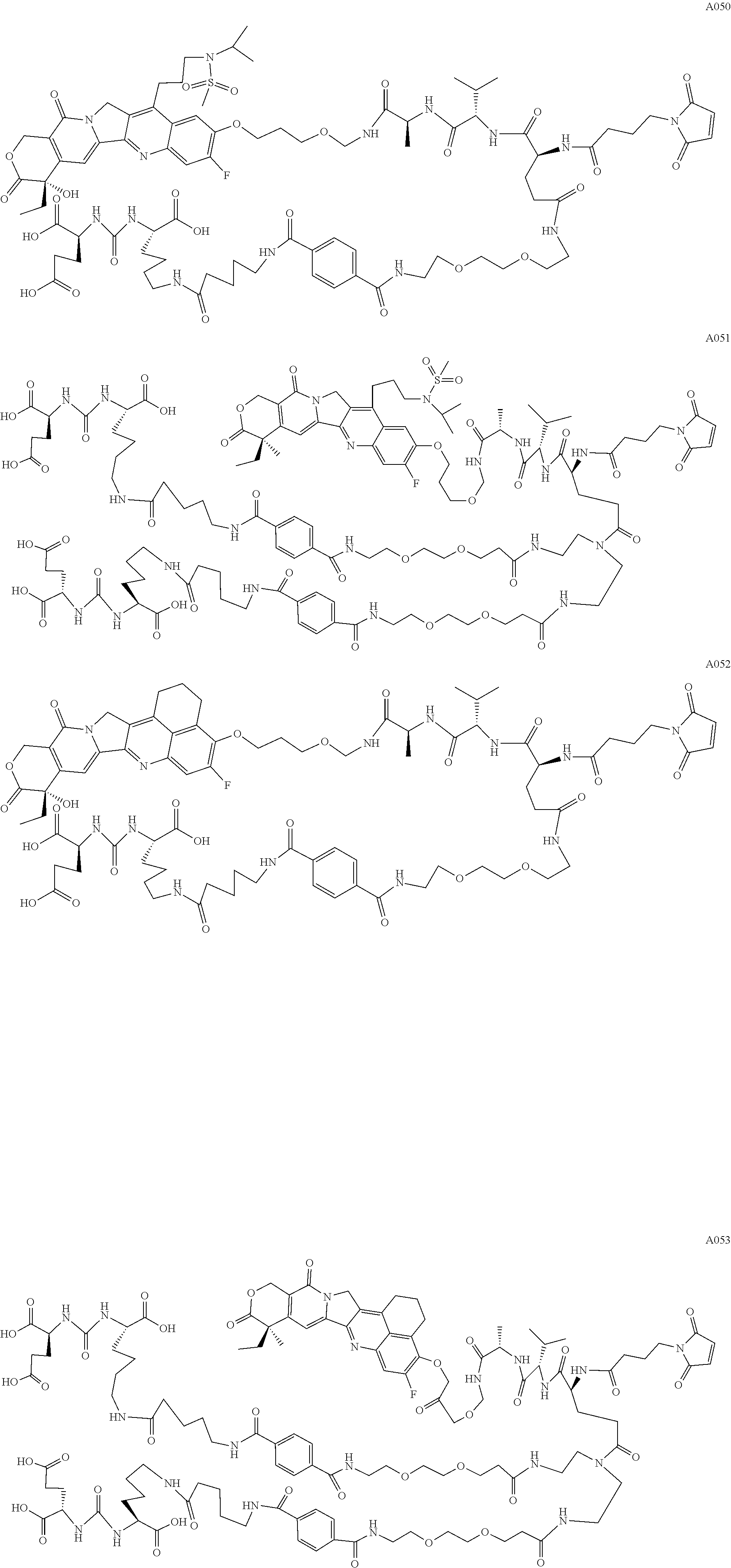

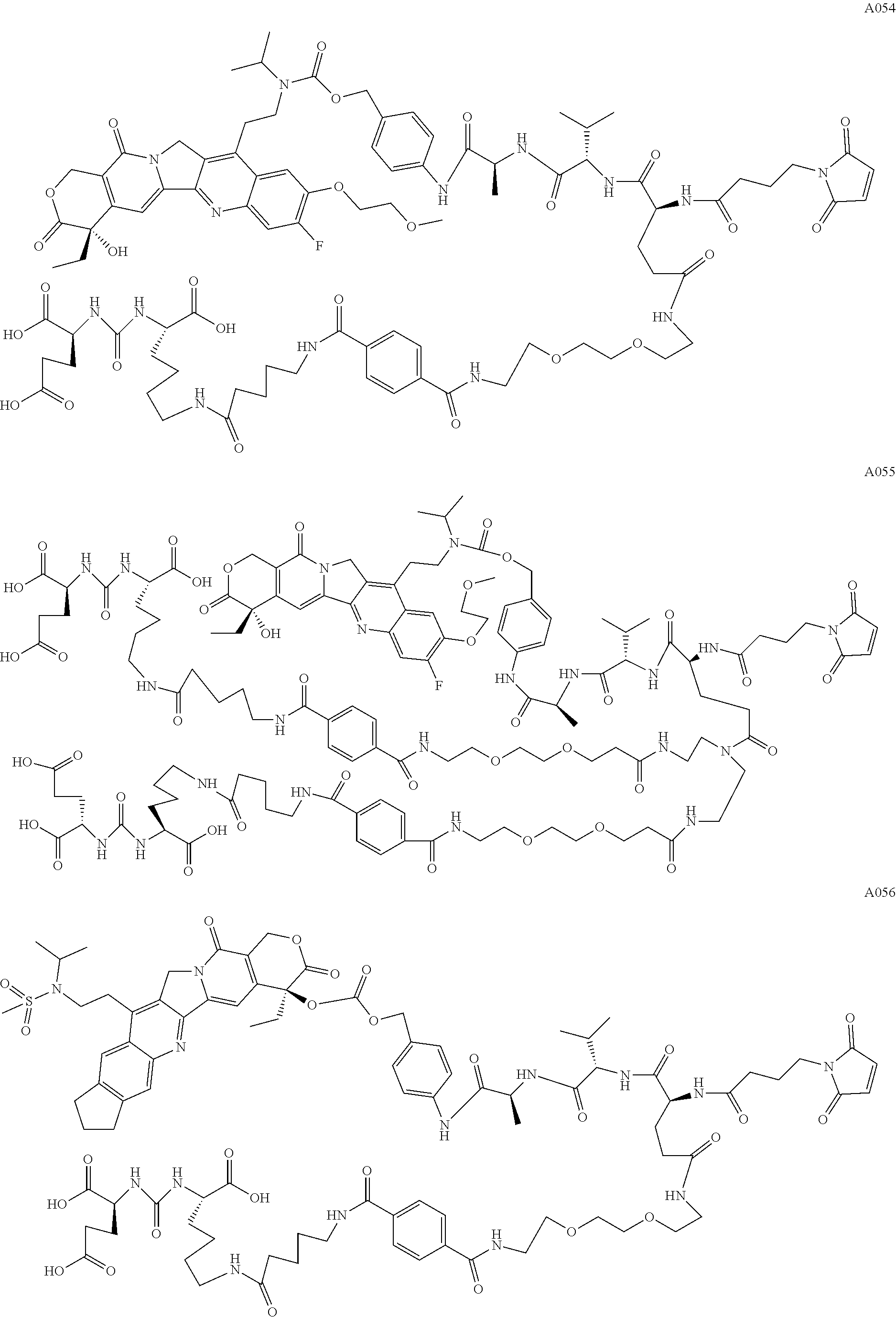

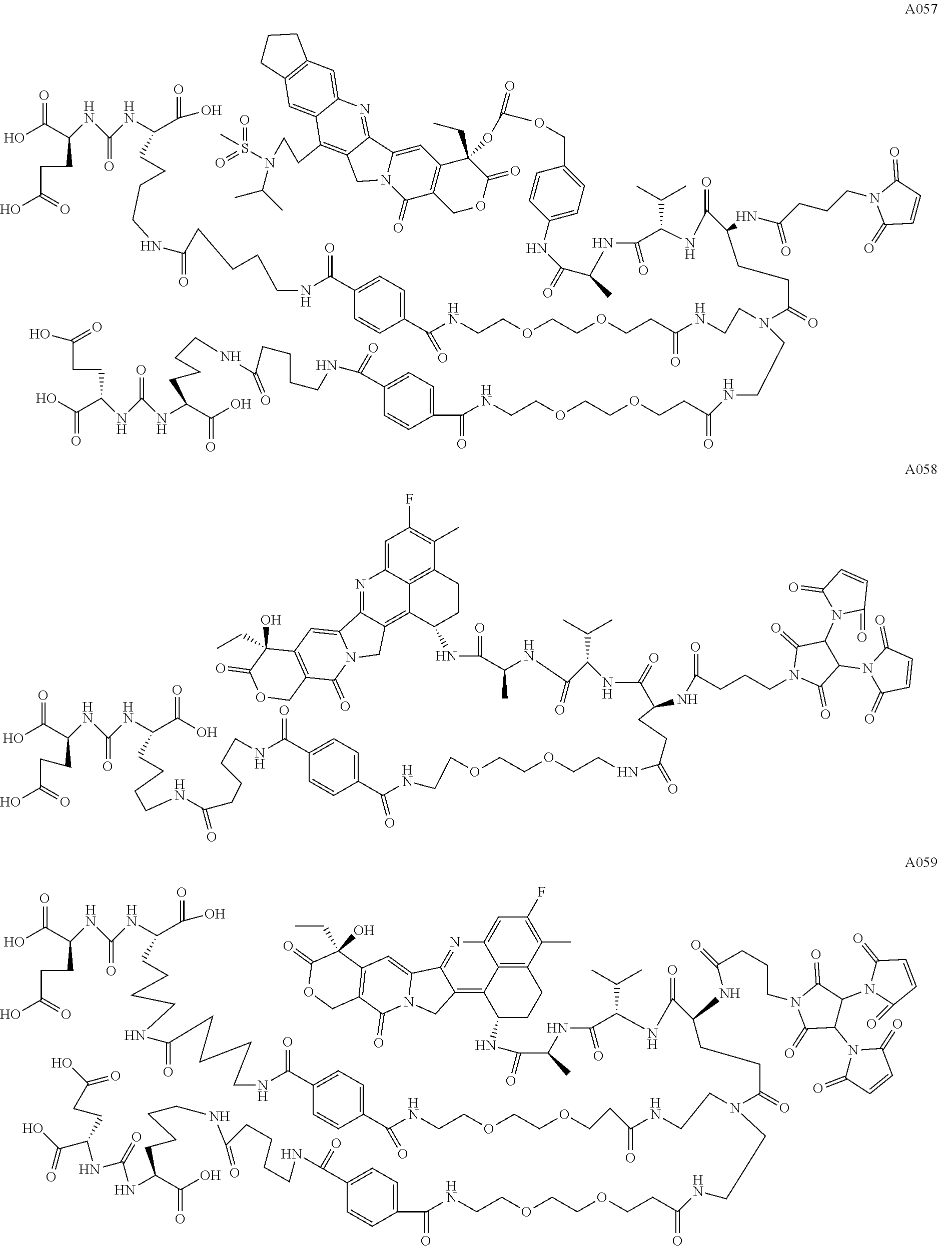

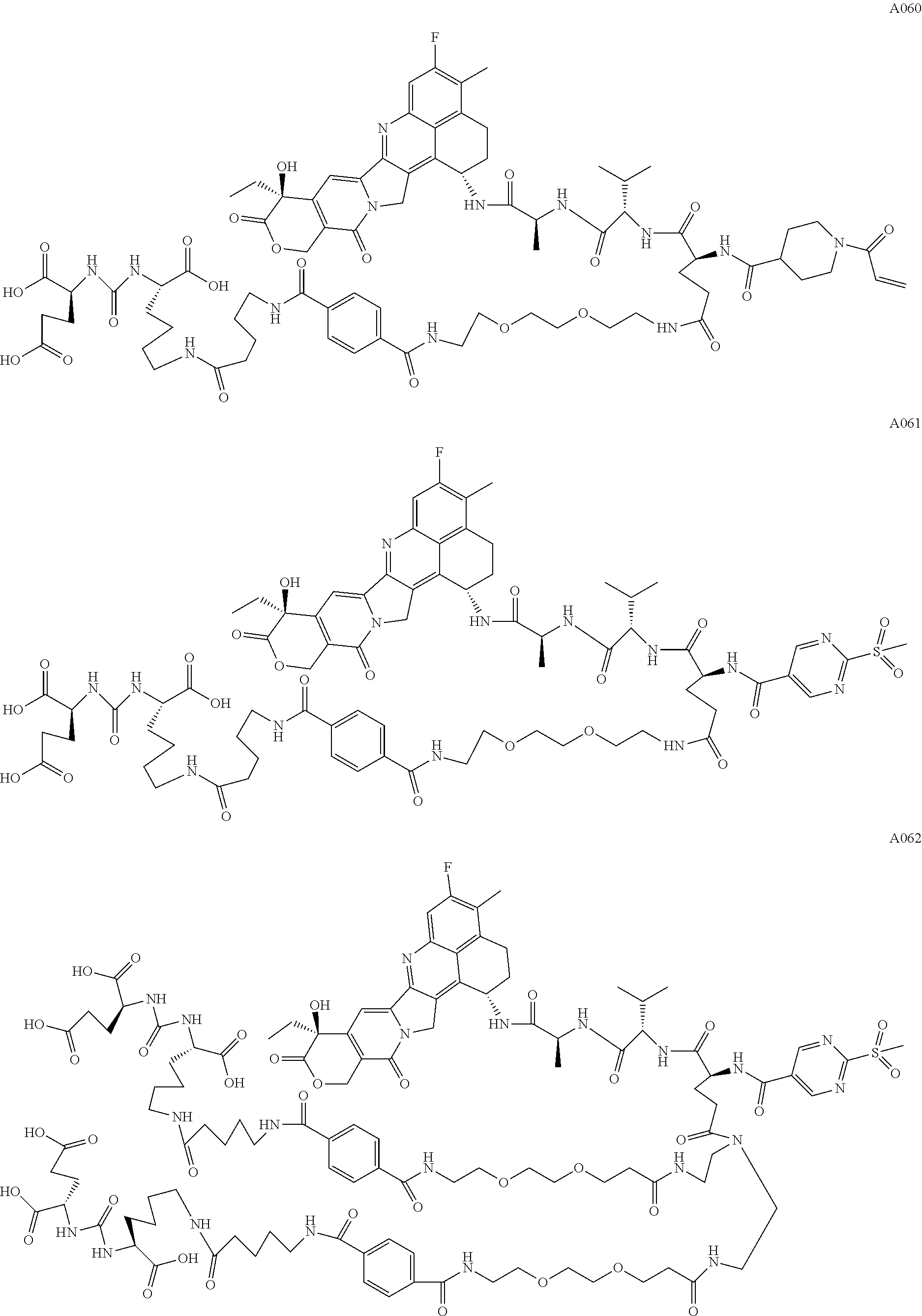

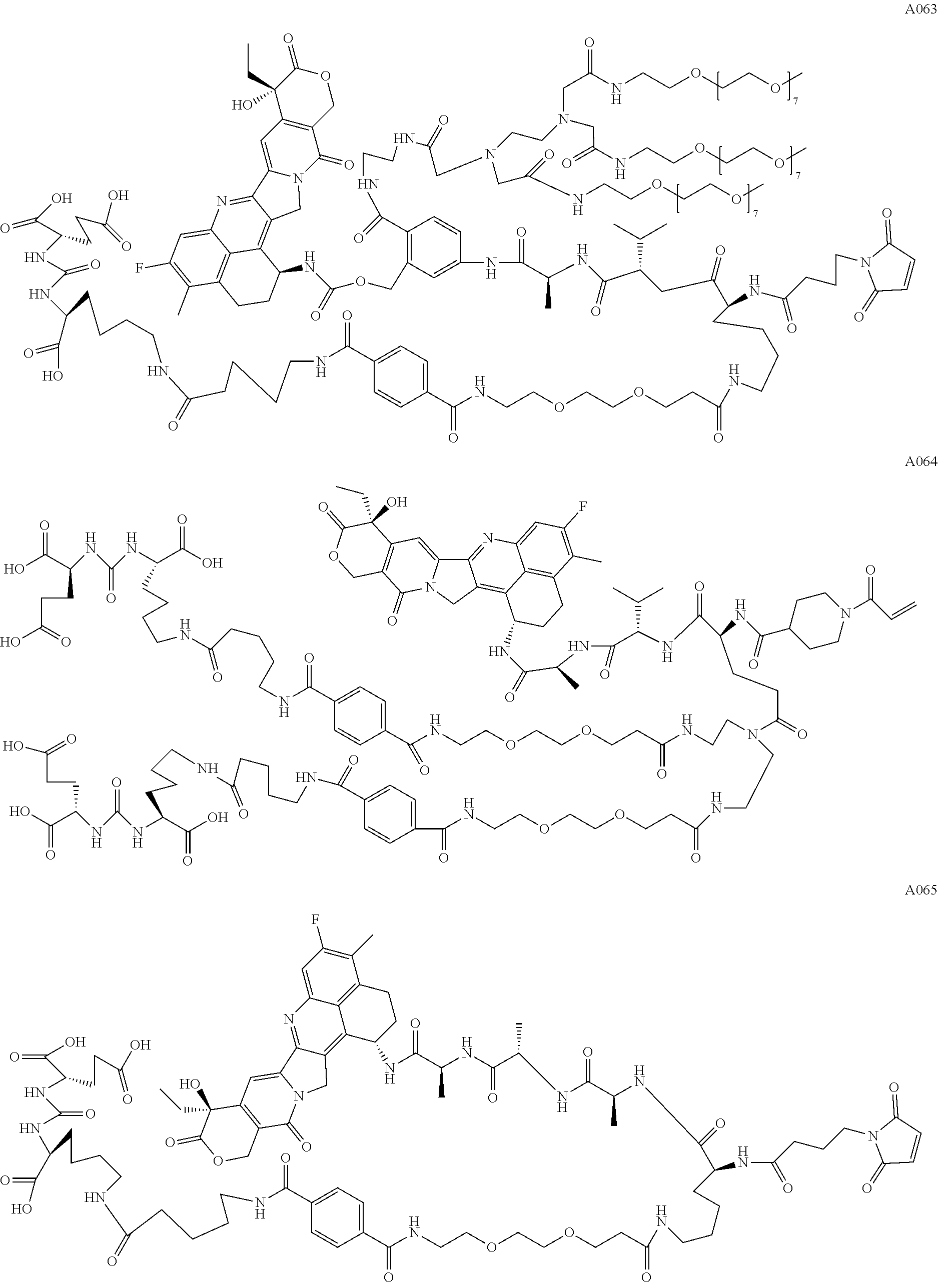

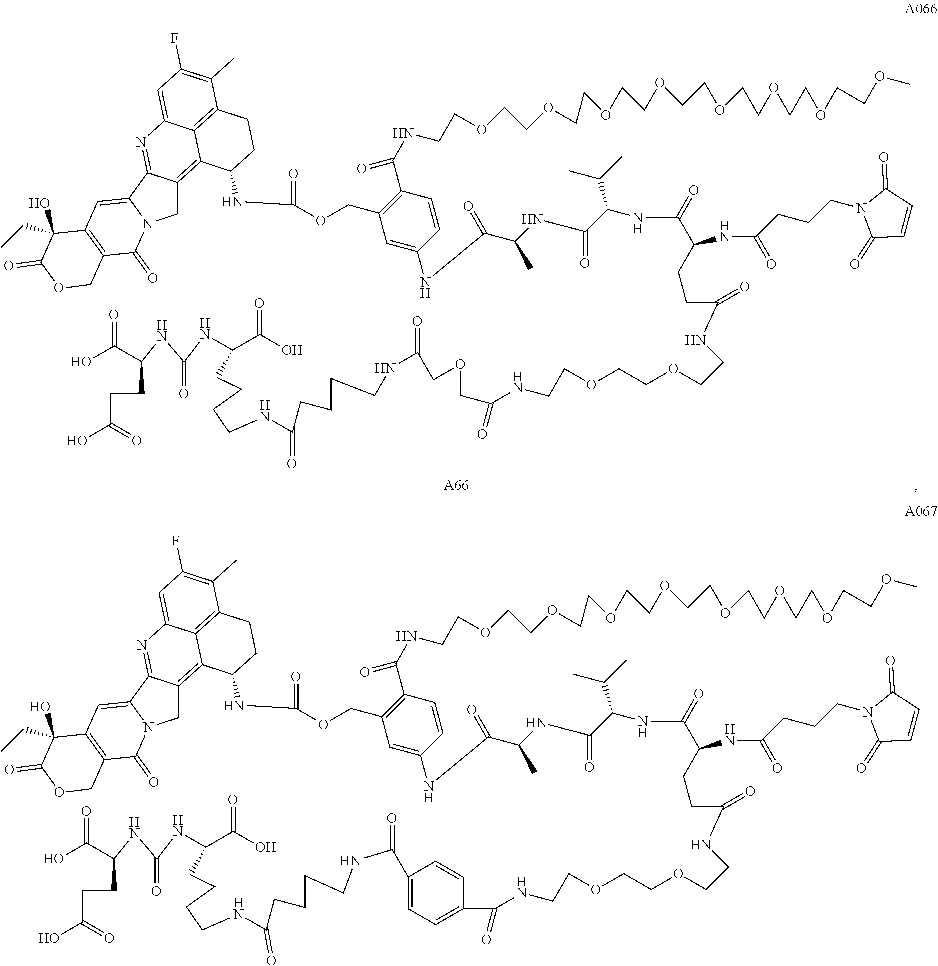

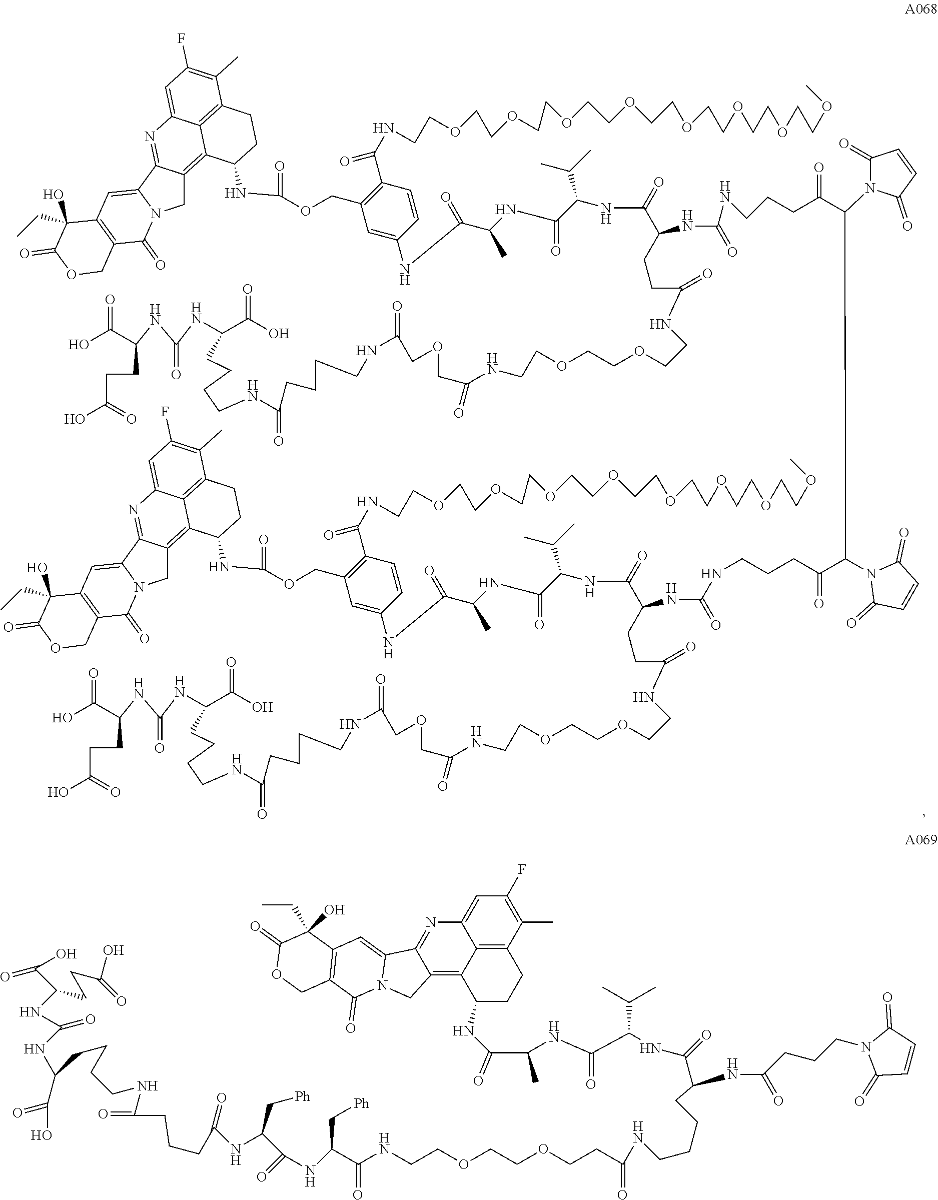

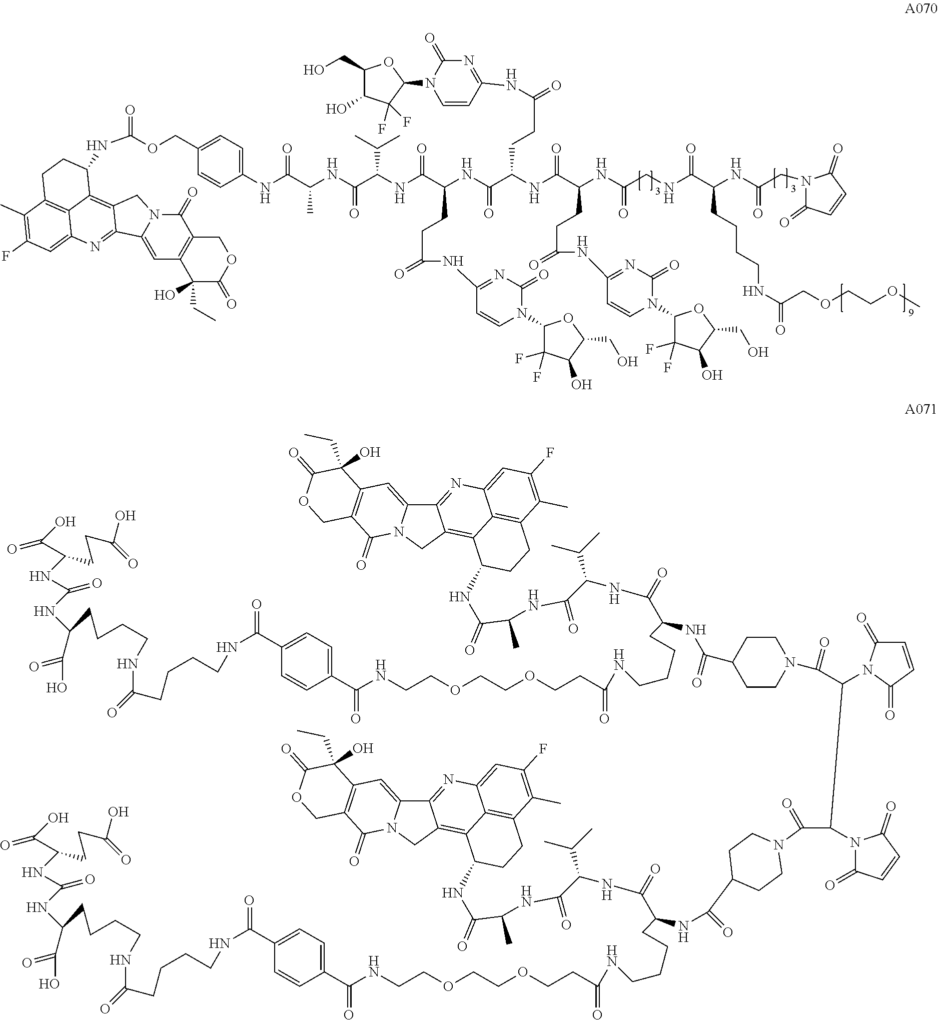

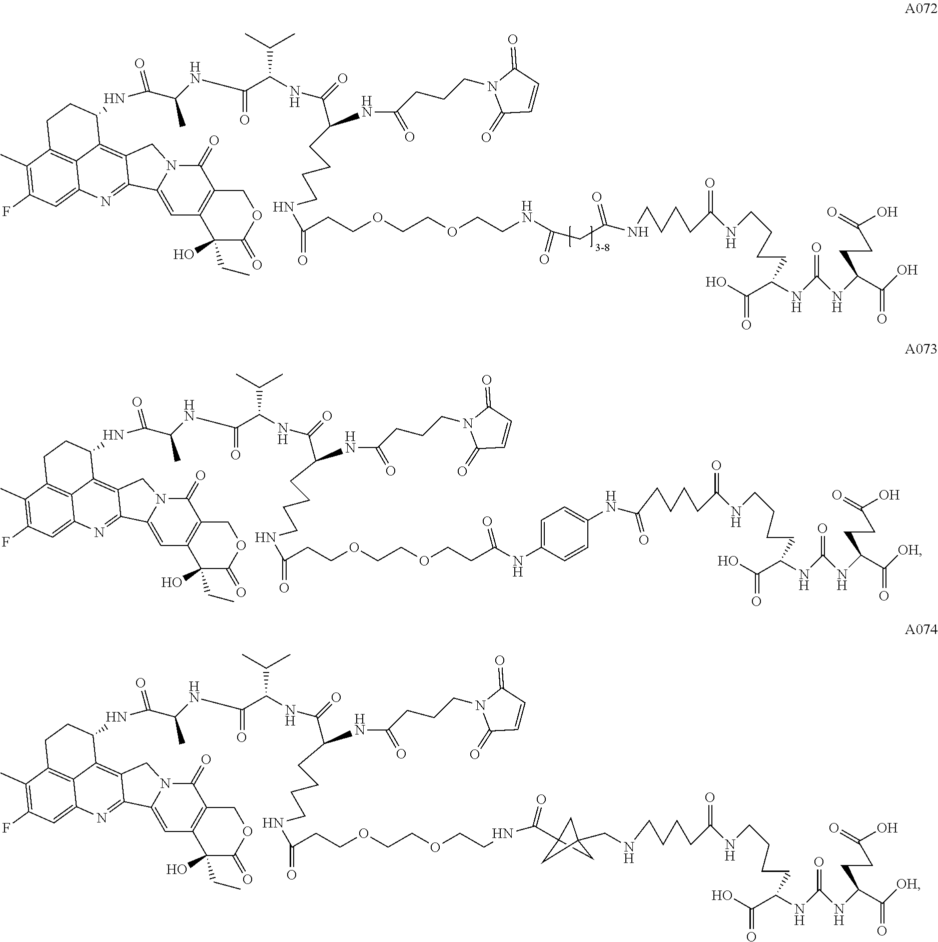

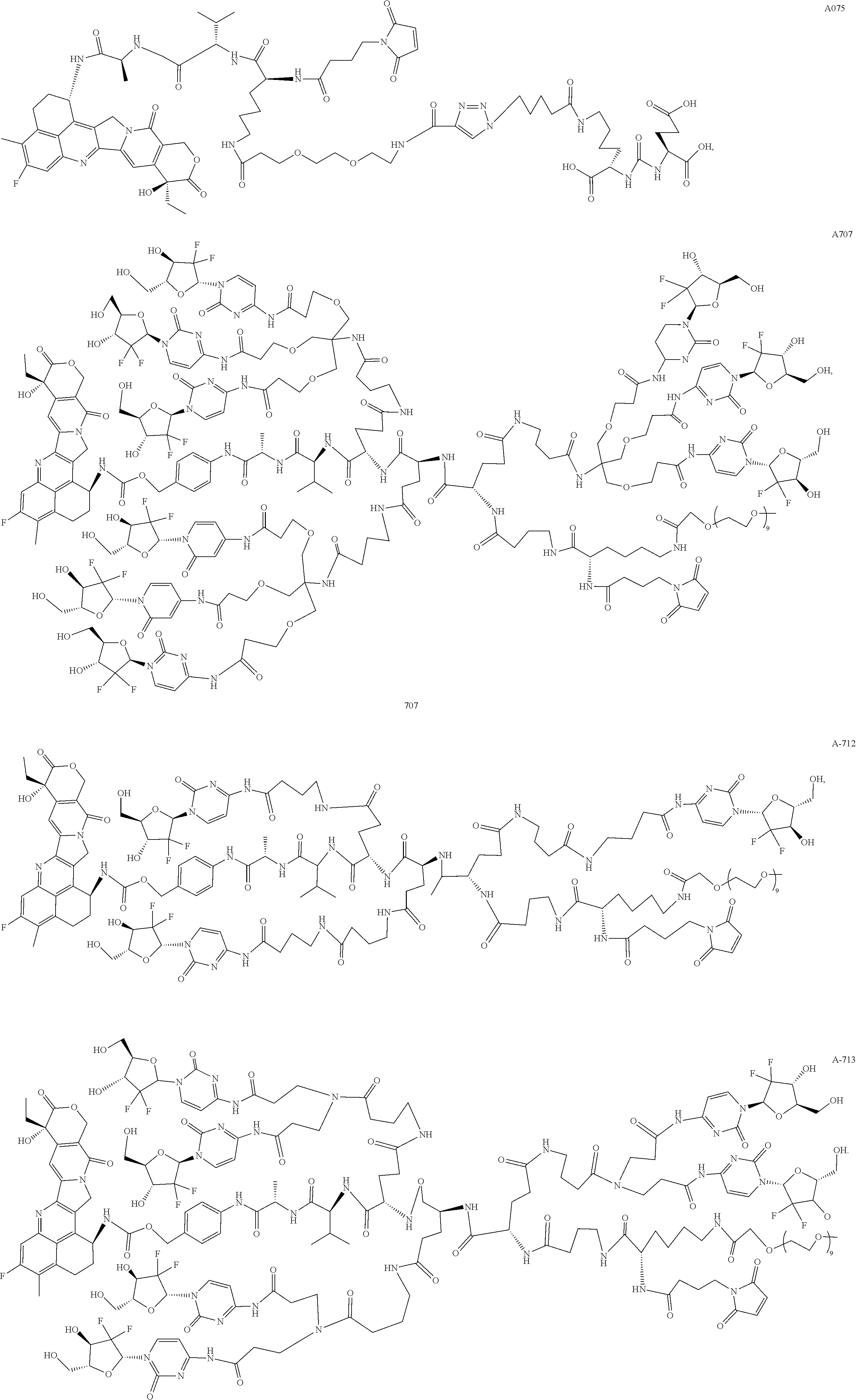

In some embodiments, the antibody drug conjugates are preferably prepared via a homogenous conjugation process, which comprises the following three key steps:
(a) incubating the antibody in the presence of an effective zinc cation-amino chelate/complex (Zn(NR₁R₂R₃)ₘ₁²⁺) and a reductant (e.g. Tris(2-carboxyethyl) phosphine (TCEP)) in a buffer system (e.g. PBS, Mes, Bis-Tris, Bis-Tris Propane, Pipes, Aces, Mopso, Bes, Mops, Hepes, Tes, Pipps, Dipso, Tapso, Heppso, Tris-up, Tris-HCl, Tricine, Hepps, Gly-Gly, Bicine, Taps, Hepee, Acetates, Histidine, Citrates, MES, or Borates, etc.) at pH 4.5˜8.5, 1˜10° C. for 1˜24 h to selectively reduce interchain disulfide bonds within the antibody, to generate thiols;
(b) introducing an effective amount of a cytotoxic drug-linker complex of formula (V), (VI) (VII) or (V′), bearing thiol reactive groups (e.g., a drug containing maleimide terminal) to react with the thiol groups resulted from step (a); For the payload/linker complex containing a disulfide bond, the reduced antibody was isolated at 2-8° C. through a chromatography (with ion exchange or size exclusion column) or dialysis prior to running conjugation reaction (addition of a cytotoxic drug-linker complex of formula (V), (VI) (VII) or (V′);
(c) adding an effective amount of oxidant (e.g. dehydroascorbic acid (DHAA)) to re-oxidize unreacted thiol groups and then purifying the resulted conjugates;
(d). the step (c) can be replaced by: adding an effective amount of cystine to quench the excessive conjugation linker or linker/payload complex containing thiol reactive groups (e.g. maleimide); and simultaneously or sequentially adding an azido compound (e.g. 4-(azidomethyl)-benzoic acid) or a disulfide compound (e.g. cystine) to quench the unreacted reductant (e.g. TCEP or Tris(hydroxypropyl)phosphine). The addition of cystine to quench the unreacted reductant (e.g. TCEP) can form a cysteine which can simultaneously quench the excessive conjugation linker or linker/payload complex of formula (V), (VI), (VII) or (VIII), containing thiol reactive groups (e. g. maleimide).

wherein R₁, R₂ and R₃ in the formula of Zn(NR₁R₂R₃)ₘ₁²⁺ are independently selected from C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; m₁ is selected from 1, 2, 3, 4, 5, 6, 7 or 8; Preferably m₁ is 1, 2, 3 or 4.

In addition, (NR₁R₂R₃) m₁ can be form a dimer, trimer, tetramer, pentamer, or hexamer wherein these polymers are covalently linked among N, R₁, R₂ and R_{3′} and N, R₁, R₂ or R₃ themselves or jointly (together) can form heterocyclic, carbocyclic, diheterocyclic, or dicarbocyclic rings.

The Zinc cation-amino chelate/complex, Zn(NR₁R₂R₃)ₘ₁²⁺, used in step (a) is 0.01 mM-1.0 mM in concentration, or 0.5˜20 equivalents in moles of the protein used, and it can be added to the reaction solution with a water-soluble organic solvent, selected from, ethanol, methanol, propanol, propandiol, DMA, DMF, DMSO, THF, CH₃CN.

The reductant is an organic phosphine, preferably selected from Tris(2-carboxyethyl)-phosphine (TECP) or Tris(hydroxypropyl) phosphine and its use in the reaction solution is 0.02 mM-1.0 mM in concentration, or 1.0-20 equivalents in moles of the protein used. The oxidant to be added in step (c) may be DHAA, Fe³⁺, I₂, Cu²⁺, Mn³⁺, MnO₂, or mixture of Fe³⁺/I⁻. The oxidant used in the reaction solution is 0.02 mM-1.0 mM in concentration, or 0.2-100 equivalents in moles of the protein used. The optimum pH in the conjugation reaction is typically between about 5.0 to 8.0, and preferably, about 5.5 to 7.5. The optimum temperature in the conjugation reaction is typically between about-5 to about 40° C., and preferably, about 0 to 37° C.; more preferably about 2 to 8° C.; further preferably about 2 to 6° C. The optimum time of the conjugation reaction is typically between about 15 min to about 48 hours and preferably, about 30 min to overnight (10˜16 h), more preferably about 2 h˜6 h. The optimal reaction conditions (e.g. pH, temperature, buffer, concentrations of the reactants) of course are depended upon specifically an antibody-like protein, a payload/linker complex, a reductant and/or Zn(NR₁R₂R₃)m₁²⁺ used.

In further embodiments, Zn(NR₁R₂R₃)ₘ₁²⁺ is preferably selected from: Zn(NH₂CH₃)₂²⁺, Zn(NH₂CH₂CH₃)₂²⁺, Zn(NH₂CH₂CH₂CH₃)₂²⁺, Zn(NH₂CH(CH₃)₂)₂²⁺, Zn(NH₂C(CH₃)₃)₂²⁺, Zn(NH₂CH₂C(CH₃)₃)₂²⁺, Zn(NH(CH₃)₂)₂²⁺, Zn(NH(CH₂CH₃)₂)₂²⁺, Zn(NH(CH(CH₃)₂)₂)₂²⁺, Zn(NH(C(CH₃)₃)₂)₂²⁺, Zn(NH(CH(CH₂CH₃)₂)₂)₂²⁺, Zn(NH(CH₂C(CH₃)₃)₂)₂²⁺, Zn(NH(CH₂C(CH₂CH₃)₃)₂)₂²⁺, Zn(NH(CH₂CH₂C(CH₃)₃)₂)₂²⁺, Zn(NH₂CH₂CH₂OH)₂²⁺, Zn(NH(CH₂CH₂OH)₂)₂²⁺, Zn(N(CH₂CH₂OH)₃)₂²⁺, Zn(NH₂CH₂COOH)₂²⁺, Zn(NH₂CH₂CONH₂)₂²⁺, Zn(NH₂CH₂COOCH₃)₂²⁺, Zn(NH₂CH₂COOCH₂CH₃)₂²⁺, Zn(NH₂CH₂COOC(CH₃)₃)₂²⁺, Zn(NH₂CH₂COOCH(CH₃)₂)₂²⁺, Zn(NH₂CH₂CH₂COOH)₂²⁺, Zn(NH(CH₂COOH)₂)₂²⁺, Zn(N(CH₂CH₂COOH)₃)₂²⁺, Zn(NH₂CH₃)₄²⁺, Zn(NH₂CH₂CH₃)₄²⁺, Zn(NH₂CH₂CH₂CH₃)₄²⁺, Zn(NH₂CH(CH₃)₂)₄²⁺, Zn(NH₂C(CH₃)₃)₄²⁺, Zn(NH₂CH₂C(CH₃)₃)₄²⁺, Zn(NH(CH₃)₂)₄²⁺, Zn(NH(CH₂CH₃)₂)₄²⁺, Zn(NH(CH(CH₃)₂)₂)₄²⁺, Zn(NH(C(CH₃)₃)₂)₄²⁺, Zn(NH(CH(CH₂CH₃)₂)₂)₄²⁺, Zn(NH(CH₂C(CH₃)₃)₂)₄²⁺, Zn(NH(CH₂C(CH₂CH₃)₃)₂)₄²⁺, Zn(NH(CH₂CH₂C(CH₃)₃)₂)₄²⁺, Zn(NH₂CH₂CH₂OH)₄²⁺, Zn(NH(CH₂CH₂OH)₂)₄²⁺, Zn(N(CH₂CH₂OH)₃)₄²⁺, Zn(NH₂CH₂COOH)₄²⁺, Zn(NH₂CH₂CONH₂)₄²⁺, Zn(NH₂CH₂COOCH₃)₄²⁺, Zn(NH₂CH₂COOCH₂CH₃)₄²⁺, Zn(NH₂CH₂COOC(CH₃)₃)₄²⁺, Zn(NH₂CH₂COOCH(CH₃)₂)₄²⁺, Zn(NH₂CH₂CH₂COOH)₄²⁺, Zn(NH(CH₂COOH)₂)₄²⁺, Zn(N(CH₂CH₂COOH)₃)₄²⁺,

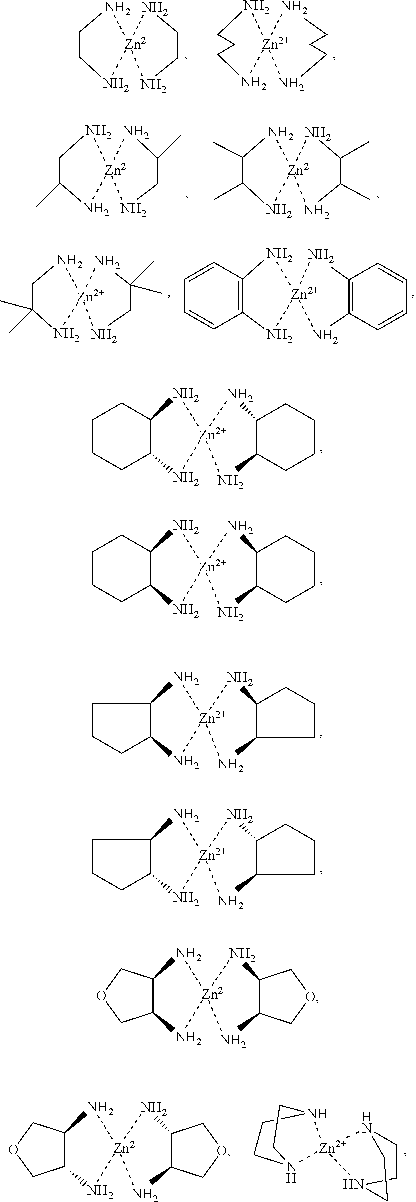

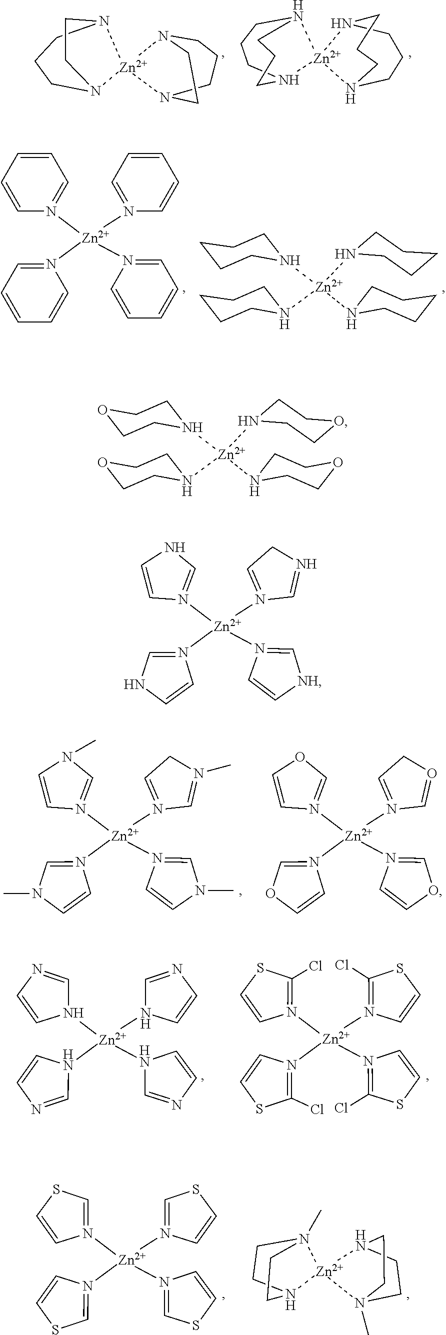

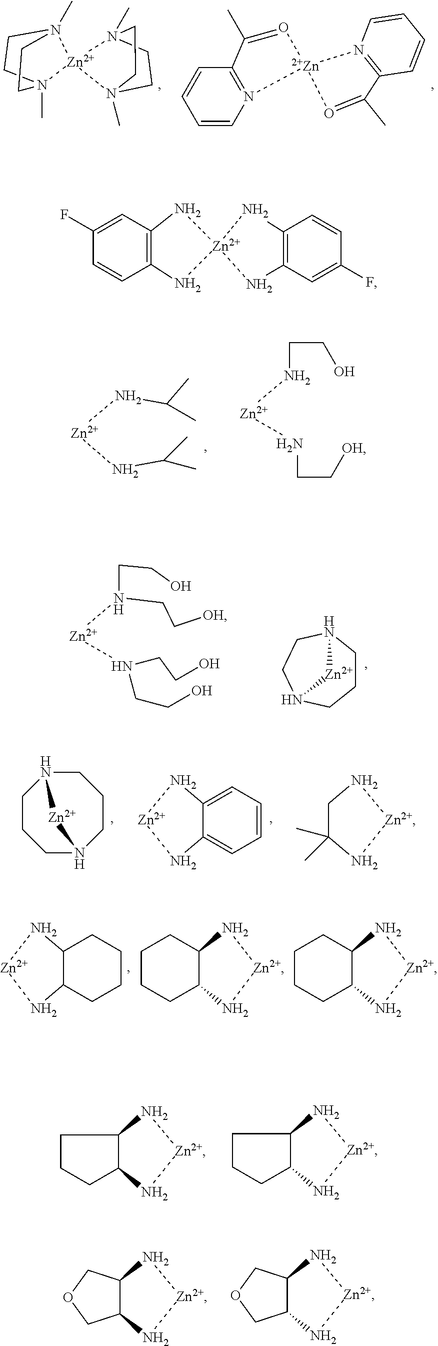

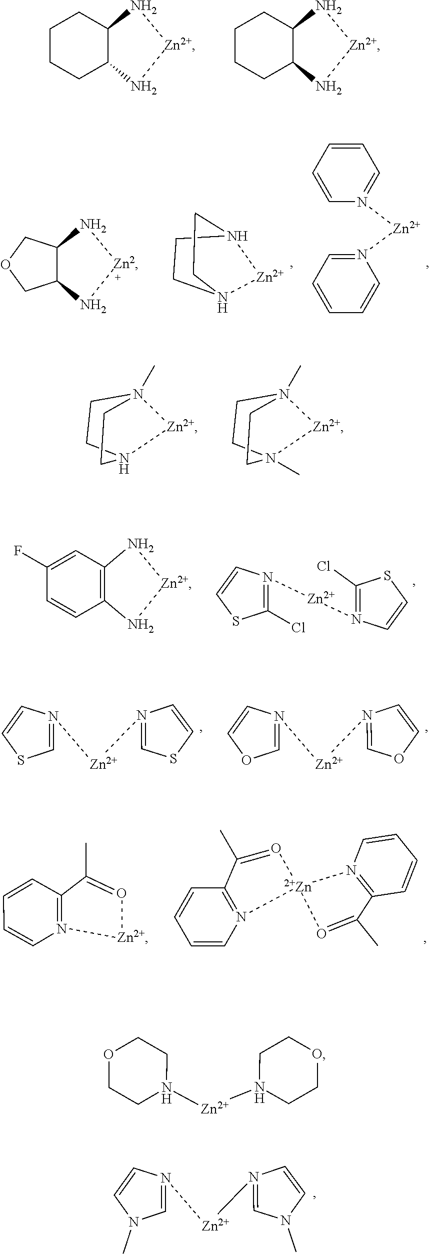

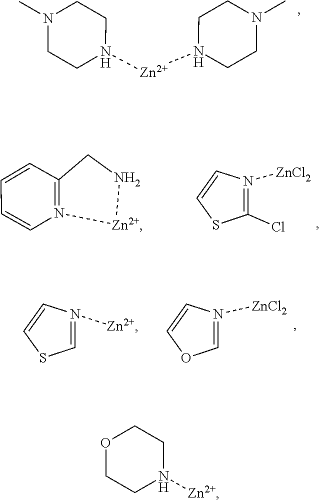

All the complex cations above can be formed with an anion, selected from, but not limited, Cl⁻, Br⁻, I⁻, SO₄²⁻, HSO₄⁻, NO₃⁻, PO₄³⁻, HPO₄²⁻, H₂PO₄⁻, CO₃²⁻, HCO₃⁻, HCOO⁻, CH₃COO⁻, F3CCOO⁻, Cl₃CCOO⁻, FCH₂COO⁻, ClCH₂COO⁻, F₂CHCOO⁻, Cl₂CHCOO⁻, BF₄, SO₃²⁻, HSO₃⁻, CH₃SO₃⁻, C₆H₅CH₂SO₃⁻, C₆H₅SO₃⁻, C₆H₅COO⁻, C₆HsCH₂COO⁻, CoFsO⁻, C₆H₄(OH)COO⁻, C₆H₂F₃O⁻, C₆H₄(NO₂)O⁻, C₆H₂(NO₂)₃O⁻, etc.

In further embodiments, under the homogenous conjugation process, the resulted conjugates of formula (I), (II), (III) or (IV) are over 75% linked to the cysteine sites between heavy-light chains of an antibody, and are less than 15% linked to the cysteine sites between heavy-heavy chains (hinge region) of an antibody. Typically, for formula (I), (II) (III) or (IV), when drug/antibody ratio (DAR) is set to be 4 and a drug linked to the linker is at one to one ratio, the distributions in percentage of the numbers of drugs in the antibody are: D0<1%, D2<10%, D4>65%, D6<10%, D8<10%; for formula (III). If the drug to linker ratio is above to 1, such as, 2, 3, 4, 5, or 6 drugs per linker (when a side chain or a multiple branched linker is used), the distributions in percentages of numbers of drugs in the antibody or antibody-like protein are increased by timing the ratios of drug/linker accordingly. The DAR can also be set up to around 6, with majority D6>65%, using both more equivalents of reducing agents, such as TCEP and more equivalents of one drug per linker payload/linker complex of formula (V), (VI), (VII) and (V′), wherein the drugs are mainly conjugated to the sites of disulfide bonds between heavy-light chains and the disulfide bonds of the upper hinge region of IgG antibodies.

In general, two steps of reaction is chosen to run conjugation of two types of payload/linker complexes containing similar maleimide groups or the other thiol reactable groups, such as making formula (IV), the first step reaction can use the homogenous conjugation reaction to conjugate the first functional payload, then the second step is to use Traut's regent or a thiol lactone to introduce a thiol through its reaction with a lysine of an antibody and then simultaneously conjugate the second functional payload/linker complex. It also can be first performed Traut's regent reaction or a thiol lactone reaction to conjugate the first functional payload/linker complex, then to conduct the second conjugation of the other functional payload/linker complex through the homogeneous conjugation reaction.

The resulted conjugates may be purified by standard biochemical means, such as gel filtration on a Sephadex G25 or Sephacryl S300 column, adsorption chromatography, ion (cation or anion) exchange chromatography, affinity chromatography (e.g. protein A column) or by dialysis (ultrafiltration or hyperfiltration (UF) and diafiltration (DF)). In some cases, a small size molecule of antibody (e.g. <100 KD) conjugated with a small molecular drugs can be purified by a chromatography such as by (reverse phase) HPLC or FPLC, size-exclusion chromatography, medium pressure column chromatography, ion exchange chromatography, or hydroxylapatite chromatography.

In further embodiments, for preparation of the conjugate of Formula (IV), the reaction of the cytotoxic drug/cytotoxic drug-linker complex of Formula (V) and (V′) to a amino acid in the antibody can be conducted at simultaneously or sequentially at the same or different conditions in the same pot. If the conjugation reactions are preformed simultaneously at the same condition, the Lv₁ of Formula (V) and Lv₂ of formula (V′) are normally differentiated. For instance, a thiol reactive group (e.g. maleimido, vinylsulfonyl, haloacetyl, acrylic, substituted propiolic) is selected for Lv₁, then an amino reactive group of N-hydoxylsuccinimidyl (NHS) ester, pentfluorophenyl ester, dinitrophenyl ester, or carboxylic acid chloride group can be chosen for Lv₂. A clickable chemistry group (e.g. azide, alkyne, dibenzocyclooctyne, BCN ((1R, 8S, 9s)-bicyclo[6.1.0]non-4-yn-9-ylmethanol)) can be selected for either Lv₁ or Lv₂ if the conjugated antibody is introduced a clickable reactive group ahead of the click chemistry reaction. Lv₁ and Lv₂ can be selected from many pairs of different function/reactive groups, such as: Amine-to-Sulfhydryl (succinimidyl (NHS) ester/maleimide, NHS ester/pyridyldithiol, NHS esters/haloacetyl), diazirine (SDA)-to-Sulfhydryl, Azide-to-Sulfhydryl, Alkyne-to-Sulfhydryl, Sulfhydryl-to-Carbohydrate (Maleimide/Hydrazide, Pyridyldithiol/Hydrazide, haloacetyl/Hydrazide), Hydroxyl-to-Sulfhydryl (Isocyanate/Maleimide), Sulfhydryl-to-DNA (Maleimide/Psoralen, Pyridyldithiol/Psoralen, haloacetyl/Psoralen), Sulfhydryl-to-Carboxyl (Carbodiimide). The conjugation reactions of Formula (V) and (V′) can also be preformed sequentially, as long as Lv₁ and Lv₂ have different reactive ability to the amino acids in the antibody/antibody like protein. For example, when both Lv₁ and Lv₂ are selected for reacting to thiols (cysteines) in the antibody/protein, Lv₁ can be maleimido, which can react to a thiol group as fast as a few seconds at pH 5,0˜ 7.5, 2.0˜37° C.; Lv₂ is then selected from slow reactive vinylsulfonyl or haloacetyl group in which the conjugation reaction with a thiol group in antibody has to be at pH>7.0, temperature over 30° C. for over 6 h.

If both Lv₁ and Lv₂ have the same (terminal) conjugatable groups, e.g., maleimido, haloacetyl or pyridyldithiol, the first conjugation with Formula (V) can be performed according to the above homogenous conjugation process, wherein the payload/linker complex is conjugated to the disulfide sites between heavy-light chains (of the Fab region) of a IgG antibody, then the Formula (V′) is adding sequentially to the reaction mixture to be conjugated at the disulfide bonds of hinge region of the IgG antibody. The first step of conjugation reaction can be conducted at low temperature, and the second step of the conjugation reaction with Formula (V′) can then be performed at room (25° C.) or higher temperature without purification of the first step reaction product as long as the compound of Formula (V′) is added in much more (3 or more) equivalents than the compound of Formula (V). The second step can also be through the reaction of Traut's reagent (2-Iminothiolane or 2-IT) with primary amines (—NH₂, normally lysine) at neutral pH (˜7.0˜7,5) to introduce sulfhydryl (—SH) groups while maintaining charge properties similar to the original amino group. Thus the introduced sulfhydryl (—SH) groups can react to a payload/linker complex containing maleimido, haloacetyl or pyridyldithiol group to generate the conjugates of Formula (IV).

When the same pH and/or temperature conditions are chosen for thioether linked conjugates under the above homogenous conjugation process, the over four times equivalents of the cytotoxic drug-linker complex containing dual terminal thiol reactive are used for the cross conjugation of disulfide bonds of heavy-light chains of an antibody. It should be noted that a preferred method of synthesis of the disulfide or thiol-ether linked conjugates can be through the first chemical synthesis the drug-linker complex having disulfide or thiol-reactive compounds of the formula (V), (VI), (VII) or (V′); following by reaction with the thiols in the protein (antibody) according the process of the invention. Synthesis of conjugates bearing an acid labile hydrazone linkage can be achieved by reaction of a carbonyl group with the hydrazide moiety in the linker, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997). Synthesis of conjugates bearing triazole linkage can be achieved by reaction of a 1-yne group of the cytotoxic drug/cytotoxic drug-linker complex or a binding ligand/binding ligand-linker complex with the azido moiety in the linker of formula (V), (VI), (VII), or (V′), through the click chemistry (Huisgen cycloaddition) (Lutz, J-F. et al, 2008, Adv. Drug Del. Rev. 60, 958-70; Sletten, E. M. et al 2011, AccChem. Research 44, 666-76). Synthesis of the conjugates linked via oxime is achieved by reaction of a modified antibody containing a ketone or aldehyde and a cytotoxic drug/cytotoxic drug-linker complex or a binding ligand/binding ligand-linker complex containing oxyamine group. A cytotoxic drug/cytotoxic drug-, or a binding ligand/binding ligand-linker complex containing an amino group can condensate with a carboxyl ester of NHS, imidazole, nitrophenoxyl; N-hydroxysuccinimide (NHS); methylsufonyl-phenoxyl; dinitrophenoxyl; pentafluorophenoxyl; tetrafluorophenoxyl; difluorophenoxyl; monofluo-rophenoxyl; pentachlorophenoxyl; triflate; imidazole; dichlorophenoxyl; tetrachlorophenoxyl; 1-hydroxybenzotriazole; tosylate; mesylate; 2-ethyl-5-phenylisoxazolium-3′-sulfonate in an antibody-linker complex to give a conjugate via amide bond linkage of Formula (I), (II), (III) or (IV). Many regular chemical and biochemical processes of the antibody-drug conjugation are known in the art (see, e.g. Matsuda, Y. and Mendelsohn, B. A., Expert Opin Biol Ther. 2021, 21 (7): 963-975; Puthenveetil, S., Methods Mol Biol. 2020, 2078:99-112; van Delft, F., and Lambert, J. M., ed. “Chemical Linkers in Antibody-Drug Conjugates (ADCs)”, Royal Soc. Chem. Pub., 22, December 2021, ISBN 978-1-83916-263-3, doi: 10.1039/9781839165153; Tumey, L. N., ed. “Antibody-Drug Conjugates, Methods and Protocols”, Springer Pub., 2020, ISBN: 978-1-4939-9929-3; Khongorzul, P. et al, Mol Cancer Res. 2020, 18(1):3-19; and many references incorporated in these books and papers). In over all, it is more preferable that the conjugates of Formula (I), (II), (III) and (IV) are directly conjugated with Formula (V), (VI), (VII), and (V) plus (V′) accordingly to an antibody in water based solution, and the compounds of Formula (V), (VI), (VII) and (V′) are constructed by chemical synthesis.

In general, the conjugate of Formula (I), (II), (III) or (IV) is preferably generated from a drug/linker complex of Formula (V), (VI), (VII), or (V) plus (V′), as in a one pot reaction. When a thiol reduced from an antibody reacts a thiol reactive group in the terminal of drug/linker complex of Formula (V), (VI), (VII), or (V′), the Ellman reagent can be optionally used to monitor the efficient reduction of the disulfide bonds and conjugation of the thiols through measurement of the numbers of the free thiols during the reactions. A UV spectrometry at wavelength of range 190-390 nm, preferably at 240-380 nm, more preferably at 240-370 nm is preferred to be used in assisting the reaction (via monitoring the conjugation). The conjugation reaction can be thus measured or conducted in a quartz cell or Pyrex flask in temperature control environment. The drug/protein (antibody) ratios (DAR) of the conjugates can also be measured by UV at wavelength of range 240-380 nm via calculation of the concentrations of the drug and the protein, by Hydrophobic Interaction Chromatography (HIC-HPLC) or Reverse Phase Chromatography (RP-HPLC) via measurement of the integration areas of each drug/protein fragment, or by Capilary electrophoresis (CE), and/or by LC-MS or LC-MS/MS or CE-MS (the combination of liquid chromatography (LC) or CE with mass spectrometry (MS) via measurement of both the integration areas of LC or CE and Peak intensity of MS for each drug/protein fragment). It is also noted in the conjugation process of the present invention, when a drug or a drug/linker complex is not well soluble in a water-based buffer solution, up to 30% of water mixable (miscible) organic solvents, such as DMA, DMF, ethanol, methanol, acetone, acetonitrile, THF, isopropanol, dioxane, propylene glycol, or ethylene diol can be added as the co-solvent in water based buffer solution.

The aqueous solutions for the modification of the antibody are buffered between pH 4 and 9, preferably between 6.0 and 7.5 and can contain any non-nucleophilic buffer salts useful for these pH ranges. Typical buffers include phosphate, acetate, triethanolamine HCl, HEPES, and MOPS buffers, which can contain additional components, such as cyclodextrins, sucrose and salts, for examples, NaCl and KCl. Other biological buffers that are used for the conjugation process are listed in the definition section. The progress of the reaction can be monitored by measuring the decrease in the absorption at a certain UV wavelength, such as at 254 nm, or increase in the absorption at a certain UV wavelength, such as 280 nm, or the other appropriate wavelength. After the reaction is complete, isolation of the modified cell-binding antibody agent can be performed in a routine way, using for example gel filtration chromatography, or adsorptive chromatography.

When disulfide exchange reaction is used for modification of the antibody, the extent of the modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by UPLC-QTOF mass spectrometry, or Capilary electrophoresis-mass spectrometry (CE-MS). The linker compounds have diverse functional groups that can react with drugs, preferably cytotoxic agents that possess a suitable substituent. For examples, the modified antibody bearing an amino or hydroxyl substituent can react with drugs bearing an N-hydroxysuccinimide (NHS) ester, the modified antibody bearing a thiol substituent can react with drugs bearing a maleimido or haloacetyl group. Additionally, the modified antibody bearing a carbonyl (ketone or aldehyde) substituent can react with drugs bearing a hydrazide or an alkoxyamine. One skilled in the art can readily determine which linker to use based on the known reactivity of the available functional group on the linkers.

### Formulation and Application

The antibody drug conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L ˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammounium, or trihydroxyethylamino salts of citric acid, ascorbic acid, gluconic acid, carbonic acid, tartaric acid, succinic acid, acetic acid or phtalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0%-20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc); etc. Preferred surfactants are polyoxyethylene sorbitan fatty acid esters e.g. polysorbate 20, 40, 60 or 80 (Tween 20, 40, 60 or 80). The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenoxyl, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0%-5.0% by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0%-30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄OH, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-β-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenoxyl, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21th edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as the antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or lyophilizate formulation should exhibit a shelf life of more than 6 months at 25° C. More preferred a conjugate in either liquid or lyophilizate formulation should exhibit a shelf life of more than 12 months at 25° C. Most preferred liquid formulation should exhibit a shelf life of about 24 to 36 months at 2-8° C. and the lyophilizate formulation should exhibit a shelf life of about preferably up to 60 months at 2-8° C. Both liquid and lyophilizate formulations should exhibit a shelf life for at least two years at −20° C., or −70° C.

In certain embodiments, the formulation is stable following freezing (e.g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS—C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e.g. 24 month, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8˜108 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜108 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 25 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

In some embodiment, when the reconstituted conjugates are injected under the skin, into a muscle, or into other tissues of the body, a hyaluronidase (HAase) is preferably administered together with the conjugates. The hyaluronidase here is used as an aid in helping patient body absorb the injected conjugates. The hyaluronidase is synergistically used 20-200 unit doses, preferably in 40-160 unit doses.

The conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/triweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferrably the unit dose range is from 1 to 500 mg administered one to four times a month and even more preferably from 1 mg to 100 mg, once a week, or once a biweek, or once a triweek. Conjugatess provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21th ed.; Lippincott Williams & Wilkins: Philadelphia, PA, 2005.

The formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination.

Liquid preparations for parenteral administration include sterile aqueous or non-aqueous solutions, suspensions, and emulsions. The liquid compositions may also include binders, buffers, preservatives, chelating agents, sweetening, flavoring, and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In yet another embodiment, a pharmaceutical composition comprising a therapeutically effective amount of the conjugate of Formula (I), (II), (III), (IV) or any conjugates described through the present patent can be administered with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The term “coadministered,” as used herein, refers to administering one or more additional therapeutic agents and the antibody or ADC described herein, or the antibody or ADC-containing composition, sufficiently close in time such that the antibody or ADC can enhance the effect of one or more additional therapeutic agents, or vice versa. In this regard, the antibody or ADC or the composition containing the same may be administered first, and the one or more additional therapeutic agents may be administered second, or vice versa. For example, the antibody or ADC or composition containing the same may be administered in combination with other agents (e.g., as an adjuvant) for the treatment or prevention of multiple myeloma. In this respect, the antibody or ADC or antibody or ADC-containing composition can be used in combination with at least one other anticancer agent including, for example, any suitable chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer), and/or surgery. The synergistic drugs or radiation therapy can be administered prior or subsequent to administration of a conjugate, in one aspect at least an hour, 12 hours, a day, a week, biweeks, triweeks, a month, in further aspects several months, prior or subsequent to administration of a conjugate of the invention.

The synergistic agents are preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, PD-1/VEGR antibody, PD-L1/VEGR antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.

In some embodiments, the disclosure also provides a composition comprising the above-described antibody or antibody-drug conjugate and a pharmaceutically acceptable (e.g., physiologically acceptable) carrier. Any suitable carrier known in the art can be used within the context of the invention. The choice of carrier will be determined, in part, by the particular site to which the composition may be administered and the particular method used to administer the composition. The composition optionally may be sterile. The compositions can be generated in accordance with conventional techniques described in, e.g., Remington: The Science and Practice of Pharmacy, 21st Edition, Lippincott Williams & Wilkins, Philadelphia, Pa. (2001).

The composition of this invention desirably comprises the antibody or ADCs in an amount that is effective to treat or prevent cancers. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the antibody or ADC or the composition comprising the antibody or ADC and a pharmaceutically acceptable carrier. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the antibody or ADC to elicit a desired response in the individual. For example, a therapeutically effective amount of the ADC of the invention is an amount which binds to a certain antigen on cancer cells and destroys them.

A pharmacologic and/or physiologic effect of treatment may be prophylactic, i.e., the effect completely or partially prevents a disease or symptom thereof. In this respect, the inventive method comprises administering a “prophylactically effective amount” of the ADC or a composition comprising the ADC to a mammal that is predisposed to multiple myeloma. A “prophylactically effective amount” refers to an amount effective, at dosages and for periods of time necessary, to achieve a desired prophylactic result (e.g., prevention of disease onset). Therapeutic or prophylactic efficacy can be monitored by periodic assessment of treated patients. In one embodiment, the ADC described herein inhibits or suppresses proliferation of prostate cancer cells by at least about 10% (e.g., at least about 20%, at least about 30%, at least about 40%, at least about 50%, at least about 60%, at least about 70%, at least about 80%, at least about 90%, or at least about 100%). Cell proliferation can be measured using any suitable method known in the art, such as measuring incorporation of labeled nucleosides (e.g., 3H-thymidine or bromodeoxyuridine Brd(U)) into genomic DNA (see, e.g., Madhavan, H. N., J. Stem Cells Regen. Med., 3 (1): 12-14 (2007)).

The invention of the ADCs further provides a method of treating a patient having or at risk of having an immune disorder mediated by immune cells expressing the antigens comprising administering to the patient an effective regime of any of the above described ADCs. Optionally, the disorder is a B cell mediated disorder. Optionally, the immune disorder is rheumatoid arthritis, systemic lupus E (SLE), Type I diabetes, asthma, atopic dermitus, allergic rhinitis, thrombocytopeni purpura, multiple sclerosis, psoriasis, Sjorgren's syndrome, Hashimoto's thyroiditis, Grave's disease, primary biliary cirrhosis, Wegener's granulomatosis, tuberculosis, and graft versus host disease.

In some embodiments, the invention of the ADCs further provides a method of treating a patient having or at risk of having a cancer, an autoimmune disease, an infectious disease, viral disease or a pathogenic infection, through administering to the patient an effective regime of any of the above ADCs, or any of the above described ADCs concurrently with the other therapeutic agents such as the chemotherapeutic agent, the radiation therapy, immunotherapy agents, autoimmune disorder agents, anti-infectious agents or the other conjugates.

The targeted cancer includes, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproli-ferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

The autoimmune disease includes, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenia purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barré syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenia purpura (See Autoimmune thrombocytopeni purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Ménière's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome.

The infectious disease includes, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuber-culosis infection, Yersiniosis, Yellow fever, Zygomycosis.

The pathogenic strain includes, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)-Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157: H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

The pathogenic viruse, includes, but not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the ADCs of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 30 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples further illustrate the invention but, of course, should not be construed as in any way limiting its scope.

### Examples

The invention is further described in the following examples, which are not intended to limit the scope of the invention. Cell lines described in the following examples were maintained in culture according to the conditions specified by the American Type Culture Collection (ATCC), Deutsche Sammlung von Mikroorganismen und Zellkulturen GmbH, Braunschweig, Germany (DMSZ), The Shanghai Cell Culture Institute of Chinese Acadmy of Science, or Nanjing Cobioer Biosciences Co., unless otherwise specified. Cell culture reagents were obtained from Invitrogen Corp., unless otherwise specified. All anhydrous solvents were commercially obtained and stored in Sure-seal bottles under nitrogen. PEG compounds were purchased from Biomatrik Inc, Jiaxing, China. Some chemical compounds, when were not referred synthesis from, were provided by CROs (e.g. Wuxi Apptec, Chemexpress, Raybow Pharma, GL Biochem, Asymchem, and Medicilin (in Hangzhou) in China. Dxd-GGFG payload/linker complex which was used for comparison with the payload/ligand/linker complexes of the present invention was purchased from Chemexpress (Shanghai). Experimental animals were purchased from National Resource Center of Model Mice via GemPharmatech. Co., Ltd, Najing, China and Shanghai SLAC Laboratory Animal Co., Ltd., Shanghai, China. All other reagents and solvents were purchased as the highest grade available and used without further purification. The preparative HPLC separations were performed with Varain PreStar HPLC. HPLC analysis was conducted on Agilent 1260. The mass spectral data were acquired on a Waters Xevo QTOF mass spectrum equipped with Waters Acquity UPLC separations module and Acquity TUV detector. NMR spectra were recorded on Zhongke-niujin WNMR-I 400 MHz instrument at the Department of Chemistry of Zhejiang Sci-Tech University. Chemical shifts (8) are reported in parts per million (ppm) referenced to tetramethylsilane at 0.00 and coupling constants (J) are reported in Hz. The elemental analysis of C, H, and/or N was provided by the Department of Chemistry of Zhejiang Sci-Tech University and conducted on Elementar UNICUBE. Quantitative analysis of metal atoms was performed on Agilent ICPOES 730 ICP-MS.

### Example 1: Synthesis of 2-(hydroxymethyl)-5-nitro-N-(2,5,8,11,14,17,20,23-octaoxapentacosan-25-yl)benzamide (3)

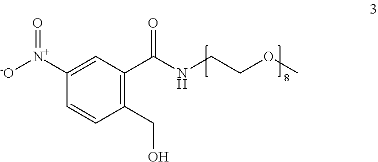

A solution of compound 1 (6.00 g, 33.500 mmol) and compound 2 (25.60 g, 67.000 mmol) in MeOH (50 mL) was stirred at room temperature for 6 h. The mixture was diluted with DCM (100 mL), washed with 5% HCl, dried over Na₂SO₄ and concentrated under vacuum to give 3 (18.40 g, 96% yield) as a yellow oil. ESI MS m/z: calcd for C₂₅H₄₂N₂O₁₂ [M+H]⁺: 563.27; found 563.28.

### Example 2: Synthesis of 5-nitro-N-(2,5,8,11,14,17,20,23-octaoxapentacosan-25-yl)-2-(((triisopropylsilyl)oxy)methyl)benzamide (4)

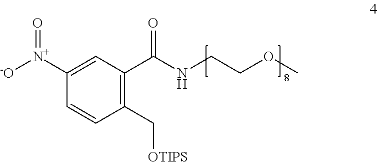

To a solution of compound 3 (18.20 g, 32.300 mmol) and chlorotriisopropylsilane (12.50 g, 64.700 mmol) in DCM (50 mL) was added imidazole (6.60 g, 97.100 mmol). After stirring for 3 h, the mixture was concentrated under vacuum. The residue was purified by flash column chromatography to give 4 (18.40 g, 79% yield) as a yellow oil. ESI MS m/z: calcd for C₃₄H₆₂N₂O₁₂Si [M+H]⁺: 719.41; found 719.50.

### Example 3: Synthesis of 5-amino-N-(2,5,8,11,14,17,20,23-octaoxapentacosan-25-yl)-2-(((triisopropylsilyl)oxy)methyl)benzamide (5)

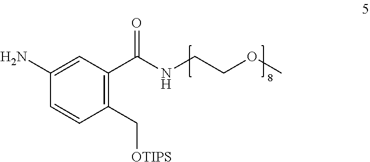

A solution of compound 4 (18.40 g, 25.600 mmol) and Pd/C (2.0 g) in ethyl acetate (200 mL) were stirred at room temperature for 20 h under H₂ protection. The mixture was filtered and the filtrate was concentrated to give 5 (17.00 g, 96% yield) as a yellow oil. ESI MS m/z: calcd for C₃₄H₆₄N₂O₁₀Si [M+H]⁺: 689.41; found 689.45.

### Example 4: Synthesis of allyl(S)-(1-((3-((2,5,8,11,14,17,20,23-octaoxapentacosan-25-yl)carbamoyl)-4-(((triisopropylsilyl)oxy)methyl)phenyl)amino)-1-oxopropan-2-yl)carbamate (7)

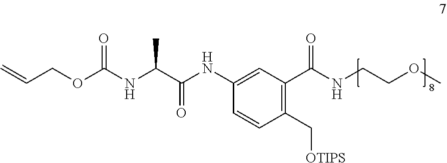

To a solution of compound 5 (17.00 g, 24.700 mmol) and compound 6 (4.70 g, 27.200 mmol) in THF (200 mL) was added EEDQ (8.00 g, 32.100 mmol). After stirred at room temperature for 30 h, the mixture was concentrated under vacuum. The residue was purified by flash column chromatography to give 7 (20.00 g, 96% yield) as a yellow oil. ESI MS m/z: calcd for C₄₁H₇₃N₃O₁₃Si [M+H]⁺: 844.49; found 844.52.

### Example 5: Synthesis of(S)-5-(2-aminopropanamido)-N-(2,5,8,11,14,17,20,23-octaoxapentacosan-25-yl)-2-(((triisopropylsilyl)oxy)methyl)benzamide (8)

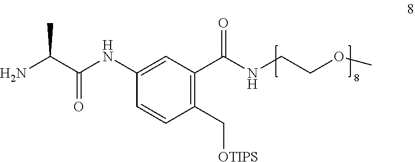

To a solution of compound 7 (9.58 g, 11.600 mmol) and pyrrolidine (5.30 g, 23.200 mmol) in DCM (100 mL) was added Pd(PPh₃)₄ (1.34 g, 1.200 mmol) under N₂ protection. After stirred at room temperature for 1 h, the mixture was concentrated under vacuum. The residue was purified by flash column chromatography to give 8 (8.50 g, 96% yield) as a yellow oil. ESI MS m/z: calcd for C₃₇H₆₉N₃O₁₁Si [M+H]⁺: 760.47; found 760.51.

### Example 6: Synthesis of(S)-5-(2-aminopropanamido)-N-(2,5,8,11,14,17,20,23-octaoxapentacosan-25-yl)-2-(((triisopropylsilyl)oxy)methyl)benzamide (10)

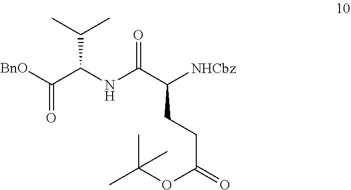 [SMILES: CC(C)[C@H](NC(=O)[C@H](CCC(=O)OC(C)(C)C)NC(=O)OCc1ccccc1)C(=O)OCc1ccccc1]

To a solution of compound 9 (10.00 g, 29.641 mmol) in DMF (100 mL) at room temperature were added benzyl L-valinate (7.22 g, 29.623 mmol), HATU (11.27 g, 29.639 mmol) and DIPEA (7.66 g, 59.265 mmol). After stirred at room temperature for 1 h, the mixture was concentrated under vacuum. The residue was purified by flash column chromatography to give 10 (18.20 g, 100% yield) as a yellow oil. ESI MS m/z: calcd for C₂₉H₃₈N₂O₇ [M+H]⁺: 527.27; found 527.27.

### Example 7: ((S)-2-amino-5-(tert-butoxy)-5-oxopentanoyl)-L-valine (11)

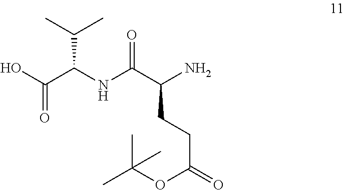 [SMILES: CC(C)[C@H](NC(=O)[C@@H](N)CCC(=O)OC(C)(C)C)C(=O)O]

To a solution of compound 10 (9.50 g, 18.039 mmol) in isopropyl alcohol (100 mL) at room temperature was added Pd/C (1.20 g, 10 wt %). The resulting mixture was stirred at room temperature under hydrogen atmosphere overnight before it was filtered through a pad of Celite. The filtrate was concentrated under vacuum. Flash column chromatography purification afforded 11 (1.80 g, 33% yield) as a yellow oil. ESI MS m/z: calcd for C₁₄H₂₆N₂O₅ [M+H]⁺: 303.18; found 303.18.

### Example 8:2,5-dioxopyrrolidin-1-yl(S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-oate (13)

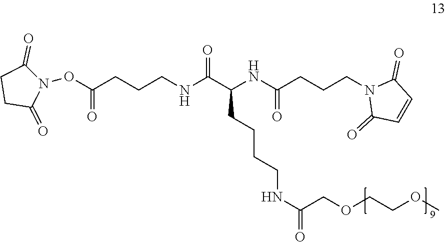

To a solution of compound 12 (6.00 g, 6.937 mmol) in DCM (8 mL) at room temperature were added NHS (0.88 g, 7.646 mmol) and EDCI (2.66 g, 13.876 mmol). The resulting mixture was stirred at room temperature for 1 h, washed with water, concentrated under vacuum, and purified by flash column chromatography to afford 13 (3.46 g, 65% yield) as a yellow oil. ESI MS m/z: calcd for C₄₃H₇₁N₅O₁₉ [M+H]⁺: 962.47; found 962.52.

### Example 9: ((S)-5-(tert-butoxy)-2-((S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-amido)-5-oxopentanoyl)-L-valine (14)

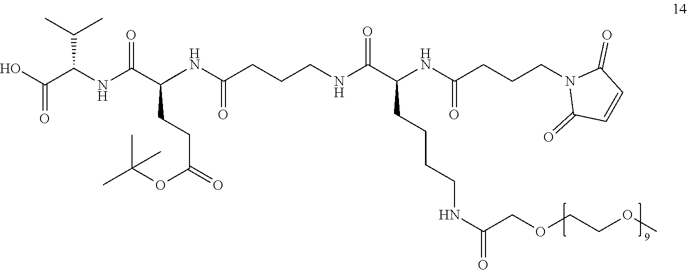

To a solution of compound 13 (1.40 g, 4.630 mmol) in DMF (50 mL) at room temperature were added compound 11 (5.35 g, 5.561 mmol) and DIPEA (0.60 g, 4.642 mmol) in sequence. The resulting mixture was stirred at room temperature for 1 h. Prep-HPLC purification and lyophilization afforded 14 (3.46 g, 65% yield) as a yellow oil. ESI MS m/z: calcd for C₅₃H₉₂N₆O₂₁ [M+H]⁺: 1149.63; found 1149.63.

### Example 10: tert-butyl (37S,45S)-45-(((S)-1-(((S)-1-((3-((2,5,8,11,14,17,20,23-octaoxapentacosan-25-yl)carbamoyl)-4-(((triisopropylsilyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oate (15)

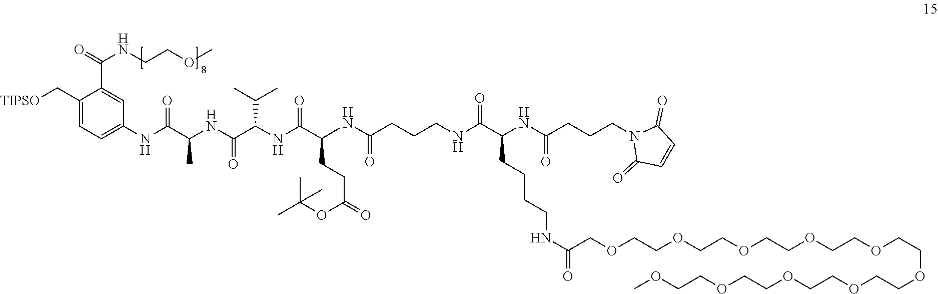

To a solution of compound 14 (2.27 g, 1.975 mmol) in DMF (2 mL) was added HATU (0.83 g, 2.173 mmol). The mixture was stirred at RT for 30 min and then compound 8 (1.50 g, 1.975 mmol) and DIPEA (1.02 g, 7.900 mmol) were added to the reaction. The mixture was stirred at RT for 0.5 h, and then purified by prep-HPLC to afford 15 (0.50 g, 13% yield) as a yellow oil. ESI MS m/z: calcd for C₉₀H₁₆₀N₉O₃₁Si [M+H]⁺: 1891.1; found 1892.0.

### Example 11: tert-butyl (37S,45S)-45-(((S)-1-(((S)-1-((3-((2,5,8,11,14,17,20,23-octaoxapentacosan-25-yl)carbamoyl)-4-(hydroxymethyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oate (16)

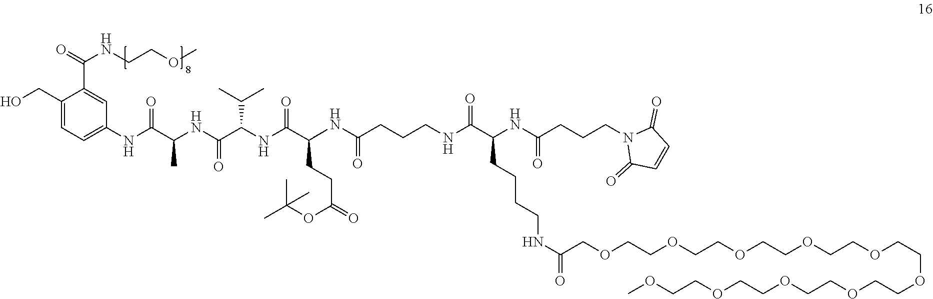

To a solution of compound 15 (350 mg, 0.185 mmol) in DCM (5 mL) was added TFA (105 mg, 0.925 mmol). The mixture was stirred at RT for 1 h, and used for the next step without purification. ESI MS m/z: calcd for C₆₇H₁₁₂N₉O₂₄ [M+H]⁺: 1735.0; found 1735.7

### Example 12: tert-butyl (37S,45S)-45-(((S)-1-(((S)-1-((3-((2,5,8,11,14,17,20,23-octaoxapentacosan-25-yl)carbamoyl)-4-((((4-nitrophenoxy)carbonyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oate (17)

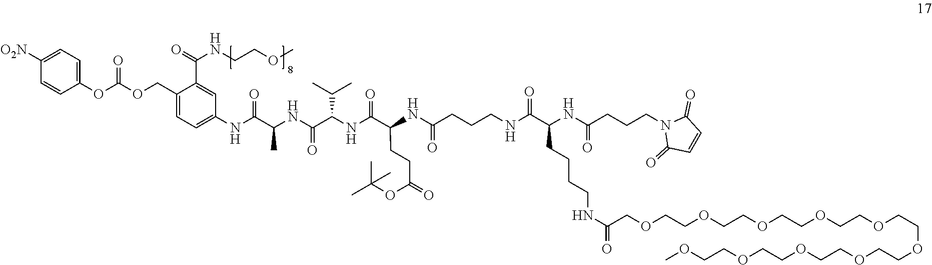

To a solution of compound 16 (321 mg, 0.185 mmol) and 4-nitrophenyl chloroformate (105 mg, 0.925 mmol) in DCM (5 mL) were added DIPEA (191 mg, 1.48 mmol) and compound 8 (68 mg, 0.222 mmol). The mixture was stirred at RT overnight and then diluted with DCM (20 mL), washed with H₂O (20 mL×3). The organic layer was dried over anhydrous Na₂SO₄ and concentrated in vacuo to afford 17 (351 mg, crude) as a yellow solid. ESI MS m/z: calcd for C₈₈H₁₄₃N₁₀O₃₅ [M+H]⁺: 1900.0; found 1900.4.

### Example 13: tert-butyl (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oate (18)

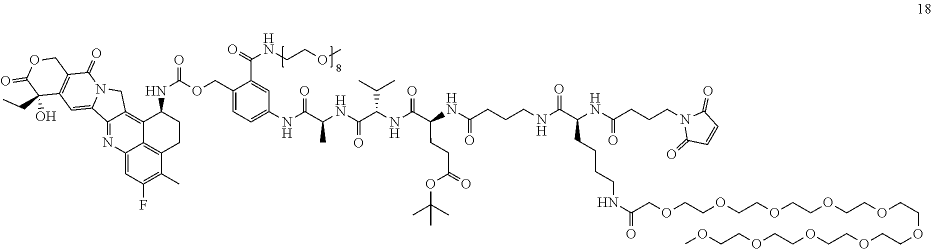

To a solution of compound 17 (325 mg, 0.171 mmol) and Exatecan (91 mg, 0.171 mmol) in DMF (2 mL) were added DIPEA (44 mg, 0.342 mmol) and HOBT (23 mg, 0.171 mmol). The mixture was stirred at RT for 6 h, and purified by prep-HPLC (H₂O/CH₃CN) to afford 18 (115 mg, 31% yield) as a yellow solid. ESI MS m/z: calcd for C₁₀₆H₁₆₀FN₁₂O₃₆ [M+H]⁺: 2196.1; found 2197.5.

### Example 14: (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oic acid (19)

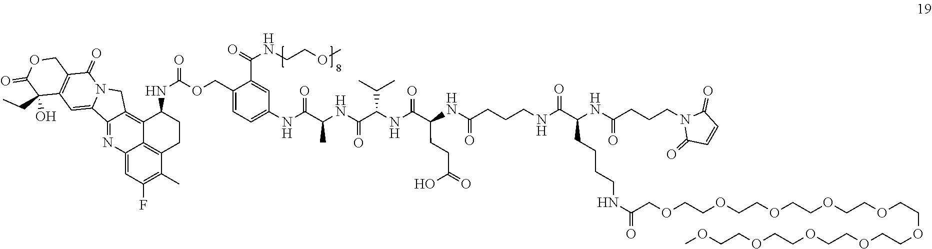

To a solution of compound 18 (76 mg, 0.035 mmol) in DCM (3 mL) was added TFA (2 mL). The mixture was stirred at RT for 0.5 h, and then concentrated in vacuo to afford 19 (74 mg, crude) as a yellow oil. ESI MS m/z: calcd for C₁₀₂H₁₅₁FN₁₂O₃₆ [M+H]⁺: 2140.04; found 2140.23.

### Example 15: perfluorophenyl (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oate (20)

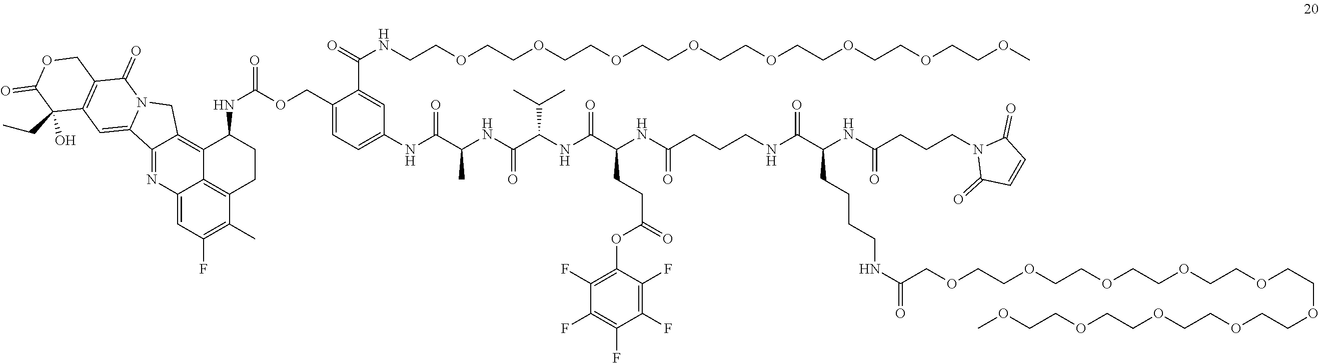 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)[C@H](CCC(=O)Oc2c(F)c(F)c(F)c(F)c2F)NC(=O)CCCNC(=O)[C@H](CCCCNC(=O)COCCOCCOCCOCCOCCOCCOCCOCCOCCOC)NC(=O)CCCN2C(=O)C=CC2=O)C(C)C)cc1C(=O)NCCOCCOCCOCCOCCOCCOCCOCCOC)CC3]

To a solution of compound 19 (1000 mg, 0.047 mmol) in DCM (10 mL) at 0° C. were added PFP—OH (100 mg, 0.056 mmol) and EDCI (18 mg, 0.09 3 mmol). The resulting mixture was stirred at 0° C. for 20 min and warmed to r.t. The mixture was stirred at room temperature for 2 h before it was quenched with H₂O. The layers were separated, and the aqueous layer was extracted with DCM. The combined organic layers were dried over Na₂SO₄ and concentrated in vacuo to afford 20 (108 mg, 100% yield) as a yellow oil. ESI MS m/z: calcd for C₁₀₈H₁₅₀F6N₁₂O₃₆ [M+H]⁺: 2305.02; found 2306.43.

### Example 16: benzyl (2S,4R)-4-(2-((1R,3R)-3-((2S,3S)-2-(2-(dimethylamino)-2-methylpropanamido)-N,3-dimethylpentanamido)-1-hydroxy-4-methylpentyl)thiazole-4-carboxamido)-2-methyl-5-phenylpentanoate (22)

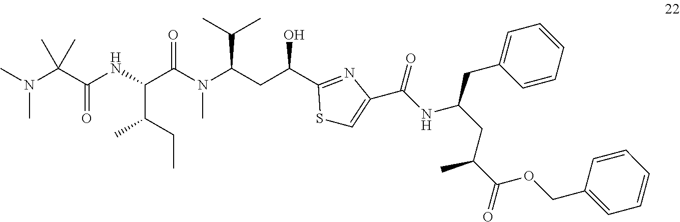 [SMILES: CC[C@H](C)[C@H](NC(=O)C(C)(C)N(C)C)C(=O)N(C)[C@H](C[C@@H](O)c1nc(C(=O)N[C@@H](Cc2ccccc2)C[C@H](C)C(=O)OCc2ccccc2)cs1)C(C)C]

To a solution of compound 21 (750 mg, 1.113 mmol) in DMF (10 mL) at room temperature were added K₂CO₃ (461 mg, 3.339 mmol), BnBr (209 mg, 1.224 mmol) and KI (92 mg, 0.556 mmol). The mixture was stirred at room temperature for 3 h before it was quenched with H₂O. The layers were separated, and the aqueous layer was extracted with EA. The organic phases were concentration, purified by flash column chromatography to afford 22 (720 mg, 85% yield) as a yellow oil. ESI MS m/z: calcd for C₄₂H₆₁N₅O₆S [M+H]⁺: 764.43; found 764.04.

### Example 17: benzyl (2S,4R)-4-(2-((6S,9R,11R,14S)-6-((S)-sec-butyl)-9-isopropyl-2,3,3,8,14,18,18-heptamethyl-4,7,13,16-tetraoxo-12,17-dioxa-2,5,8,15-tetraazanonadecan-11-yl)thiazole-4-carboxamido)-2-methyl-5-phenylpentanoate (23)

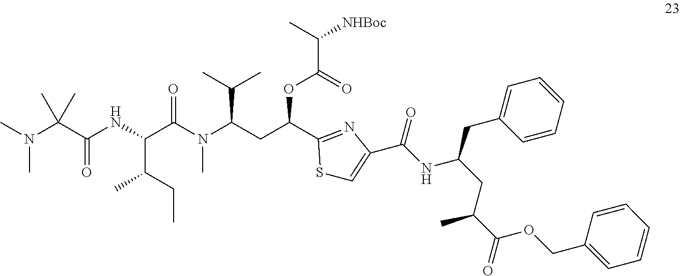 [SMILES: CC[C@H](C)[C@H](NC(=O)C(C)(C)N(C)C)C(=O)N(C)[C@H](C[C@@H](OC(=O)[C@H](C)NC(=O)OC(C)(C)C)c1nc(C(=O)N[C@@H](Cc2ccccc2)C[C@H](C)C(=O)OCc2ccccc2)cs1)C(C)C]

To a solution of compound 22 (550 mg, 0.720 mmol) in DCM (10 mL) were added N—BOC-L-Alanine (164 mg, 0.864 mmol), DCC (298 mg, 1.440 mmol) and 4-PPy (53 mg, 0.360 mmol) at 0° C. The resulting mixture was stirred at 0° C. for 20 min and warmed to RT. After stirring for 2 h, the reaction was filtered, the filtrate was concentrated in vacuo. Flash column chromatography purification afforded 23 (552 mg, 82% yield) as a yellow oil. ESI MS m/z: calcd for C₅₀H₇₄N₆O₉S [M+H]⁺: 935.52; found 935.24.

### Example 18: benzyl (2S,4R)-4-(2-((6S,9R,11R,14S)-14-amino-6-((S)-sec-butyl)-9-isopropyl-2,3,3,8-tetramethyl-4,7,13-trioxo-12-oxa-2,5,8-triazapentadecan-11-yl)thiazole-4-carboxamido)-2-methyl-5-phenylpentanoate (24)

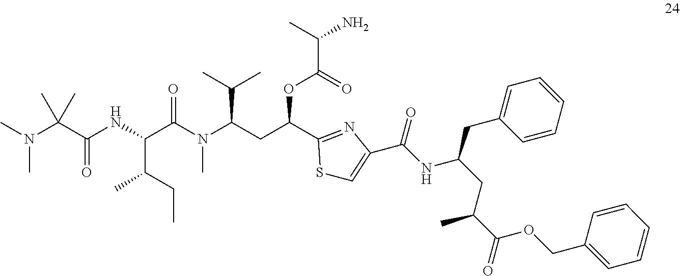 [SMILES: CC[C@H](C)[C@H](NC(=O)C(C)(C)N(C)C)C(=O)N(C)[C@H](C[C@@H](OC(=O)[C@H](C)N)c1nc(C(=O)N[C@@H](Cc2ccccc2)C[C@H](C)C(=O)OCc2ccccc2)cs1)C(C)C]

To a solution of compound 23 (550 mg, 0.588 mmol) in DCM (5 mL) at room temperature was added 4M HCl/EA (3 mL). The resulting mixture was stirred at room temperature for 3 h before it was concentrated in vacuo to afford 24 (562 mg, 100% yield) as a yellow oil. ESI MS m/z: calcd for C₄₅H₆₆N₆O₇S [M+H]⁺: 835.47; found 835.12

### Example 19: benzyl (2S,4R)-4-(2-((6S,9R,11R,14S,17S)-6-((S)-sec-butyl)-9-isopropyl-2,3,3,8,14,17,21,21-octamethyl-4,7,13,16,19-pentaoxo-12,20-dioxa-2,5,8,15,18-pentaazadocosan-11-yl)thiazole-4-carboxamido)-2-methyl-5-phenylpentanoate (25)

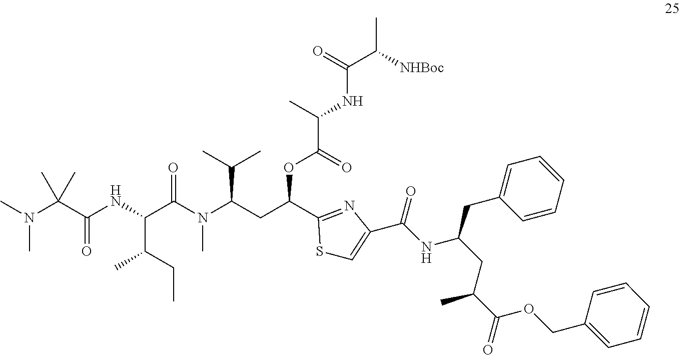 [SMILES: CC[C@H](C)[C@H](NC(=O)C(C)(C)N(C)C)C(=O)N(C)[C@H](C[C@@H](OC(=O)[C@H](C)NC(=O)[C@H](C)NC(=O)OC(C)(C)C)c1nc(C(=O)N[C@@H](Cc2ccccc2)C[C@H](C)C(=O)OCc2ccccc2)cs1)C(C)C]

To a solution of compound 24 (562 mg, 0.588 mmol) in DCM (10 mL) at 0° C. were added N-Boc-L-Alanine (111 mg, 0.588 mmol), HATU (246 mg, 0.647 mmol) and DIEA (52 mg, 1.176 mmol). The resulting mixture was stirred at 0° C. for 20 min, warmed to RT, stirred for 1 h and quenched with H₂O. The layers were separated, and the aqueous layer was extracted with DCM. The organic phase was concentrated and purified by prep-HPLC to afford 25 (562 mg, 100% yield) as a yellow oil. ESI MS m/z: calcd for C₅₃H₇₉N₇O₁₀S [M+H]⁺: 1006.56; found 1006.31

### Example 20: (2S,4R)-4-(2-((6S,9R,11R,14S,17S)-6-((S)-sec-butyl)-9-isopropyl-2,3,3,8,14,17,21,21-octamethyl-4,7,13,16,19-pentaoxo-12,20-dioxa-2,5,8,15,18-pentaazadocosan-11-yl)thiazole-4-carboxamido)-2-methyl-5-phenylpentanoic acid (26)

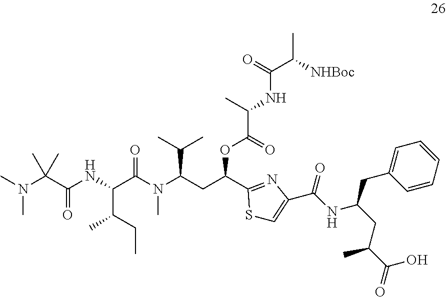 [SMILES: CC[C@H](C)[C@H](NC(=O)C(C)(C)N(C)C)C(=O)N(C)[C@H](C[C@@H](OC(=O)[C@H](C)NC(=O)[C@H](C)NC(=O)OC(C)(C)C)c1nc(C(=O)N[C@@H](Cc2ccccc2)C[C@H](C)C(=O)O)cs1)C(C)C]

To a solution of compound 25 (220 mg, 0.219 mmol) in MeOH (20 mL) at room temperature was added Pd(OH)₂ (40 mg, 10 wt %). The resulting mixture was stirred at room temperature under hydrogen atmosphere for 18 h before it was filtered through a pad of Celite. The filtrate was concentrated in vacuo, purified by prep-HPLC to afford 26 (88 mg, 44% yield) as a white solid. ESI MS m/z: calcd for C₄₆H₇₃N₇O₁₀S [M+H]⁺: 916.51; found: 916.19.

### Example 21: (2S,4R)-4-(2-((6S,9R,11R,14S,17S)-17-amino-6-((S)-sec-butyl)-9-isopropyl-2,3,3,8,14-pentamethyl-4,7,13,16-tetraoxo-12-oxa-2,5,8,15-tetraazaoctadecan-11-yl)thiazole-4-carboxamido)-2-methyl-5-phenylpentanoic acid (27)

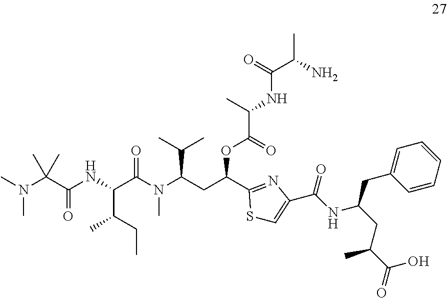 [SMILES: CC[C@H](C)[C@H](NC(=O)C(C)(C)N(C)C)C(=O)N(C)[C@H](C[C@@H](OC(=O)[C@H](C)NC(=O)[C@H](C)N)c1nc(C(=O)N[C@@H](Cc2ccccc2)C[C@H](C)C(=O)O)cs1)C(C)C]

To a solution of compound 26 (80 mg, 0.088 mmol) in DCM (5 mL) at room temperature was added TFA (1 mL). The resulting mixture was stirred at room temperature for 1 h before it was concentrated in vacuo to afford 27 (76 mg, 100% yield) as a white solid. ESI MS m/z: calcd for C₄₁H₆₅N₇O₈S [M+H]⁺: 816.46; found 816.07.

### Example 22: (2S,4R)-4-(2-((37S,45S,48S,51R,53R,56S)-56-((S)-sec-butyl)-37-(4-(2,5-dioxo-2,5-dihydro-1H-pyrrol-1-yl) butanamido)-53-isopropyl-45,48,54,59,59,60-hexamethyl-31,38,43,46,49,55,58-heptaoxo-2,5,8,11,14,17,20,23,26,29,50-undecaoxa-32,39,44,47,54,57,60-heptaazahenhexacontan-51-yl)thiazole-4-carboxamido)-2-methyl-5-phenylpentanoic acid (28)

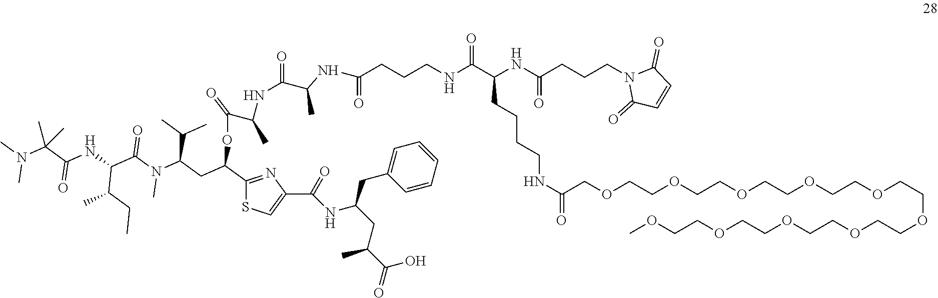 [SMILES: CC[C@H](C)[C@H](NC(=O)C(C)(C)N(C)C)C(=O)N(C)[C@H](C[C@@H](OC(=O)[C@H](C)NC(=O)[C@H](C)NC(=O)CCCNC(=O)[C@H](CCCCNC(=O)COCCOCCOCCOCCOCCOCCOCCOCCOCCOC)NC(=O)CCCN1C(=O)C=CC1=O)c1nc(C(=O)N[C@@H](Cc2ccccc2)C[C@H](C)C(=O)O)cs1)C(C)C]

To a solution of compound 13 (45 mg, 0.046 mmol) in DMF (3 mL) at 0° C. were added compound 6 (36 mg, 0.044 mmol) and DIEA (16.0 μL, 0.094 mmol). The mixture was stirred at 0° C. for 20 min, warmed to RT, stirred for 1 h and directly purified by prep-HPLC to afford 28 (77 mg, 45% yield) as a white solid. ESI MS m/z: calcd for C₈₀H₁₃₁N₁₁O₂₄S [M+H]⁺: 1662.91; found 1663.04.

### Example 23: (2S,4R)-4-(2-((37S,45S,50S,53S,56R,58R,61S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy) methyl)phenyl)amino)-1-oxopropan-2-yl)amino)-3-methyl-1-oxobutan-2-yl)carbamoyl)-61-((S)-sec-butyl)-37-(4-(2,5-dioxo-2,5-dihydro-1H-pyrrol-1-yl) butanamido)-58-isopropyl-50,53,59,64,64,65-hexamethyl-31,38,43,48,51,54,60,63-octaoxo-2,5,8,11,14,17,20,23,26,29,55-undecaoxa-32,39,44,49,52,59,62,65-octaazahexahexacontan-56-yl)thiazole-4-carboxamido)-2-methyl-5-phenylpentanoic acid (29)

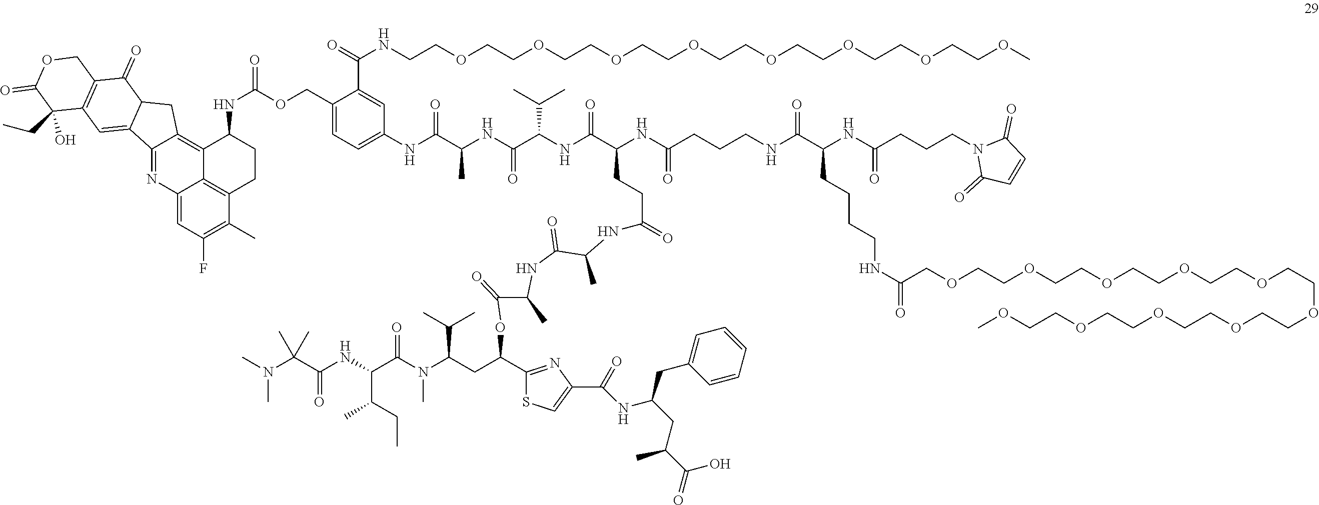 [SMILES: CC[C@H](C)[C@H](NC(=O)C(C)(C)N(C)C)C(=O)N(C)[C@H](C[C@@H](OC(=O)[C@H](C)NC(=O)[C@H](C)NC(=O)CC[C@H](NC(=O)CCCNC(=O)[C@H](CCCCNC(=O)COCCOCCOCCOCCOCCOCCOCCOCCOCCOC)NC(=O)CCCN1C(=O)C=CC1=O)C(=O)N[C@H](C(=O)N[C@@H](C)C(=O)Nc1ccc(COC(=O)N[C@H]2CCc3c(C)c(F)cc4nc5c(c2c34)CC2C(=O)C3=C(C=C52)[C@@](O)(CC)C(=O)OC3)c(C(=O)NCCOCCOCCOCCOCCOCCOCCOCCOC)c1)C(C)C)c1nc(C(=O)N[C@@H](Cc2ccccc2)C[C@H](C)C(=O)O)cs1)C(C)C]

To a solution of compound 13 (108 mg, 0.047 mmol) in DMF (3 mL) at 0° C. were added compound 28 (36 mg, 0.044 mmol) and DIEA (16.0 μL, 0.094 mmol). The mixture was stirred at 0° C. for 20 min and warmed to r.t. The mixture was stirred at room temperature for 30 min before it was purified by prep-HPLC to afford 29 (71 mg, 51% yield) as a white solid. ESI MS m/z: calcd for C₁₄₃H₂₁₄FN₁₉O₄₃S [M+2H]²⁺: 1469.24; found 1469.35.

### Example 23: perfluorophenyl 2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxylate (31)

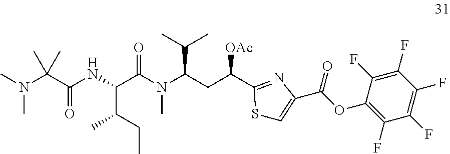 [SMILES: CC[C@H](C)[C@H](NC(=O)C(C)(C)N(C)C)C(=O)N(C)[C@H](C[C@@H](OC(C)=O)c1nc(C(=O)Oc2c(F)c(F)c(F)c(F)c2F)cs1)C(C)C]

To a solution of compound 21 (227 mg, 0.430 mmol) in DCM (10 mL) at 0° C. were added PFP—OH (118 mg, 0.650 mmol) and EDCI (165 mg, 0.860 mmol). The resulting mixture was stirred at 0° C. for 20 min and warmed to r.t. The mixture was stirred at room temperature for 2 h before it was quenched with H₂O. The layers were separated, and the aqueous layer was extracted with CH₂Cl₂. The combined organic layers were dried over Na₂SO₄ and concentrated in vacuo to afford 31 (300 mg, 51% yield) as a white solid. ESI MS m/z: calcd for C₃₁H₄₁F₅N₄O₆S [M+H]⁺: 693.27; found 693.27.

### Example 24: 1-(((2S,3S)-1-(((1R,3R)-1-acetoxy-4-methyl-1-(4-((perfluorophenoxy)carbonyl)thiazol-2-yl) pentan-3-yl)(methyl)amino)-3-methyl-1-oxopentan-2-yl)amino)-N-(4-((S)-2-((tert-butoxycarbonyl)amino)propanamido)benzyl)-N,N,2-trimethyl-1-oxopropan-2-aminium (33)

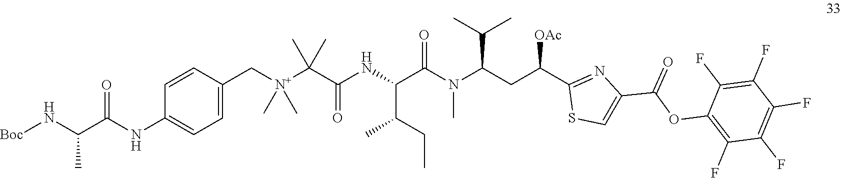 [SMILES: CC[C@H](C)[C@H](NC(=O)C(C)(C)[N+](C)(C)Cc1ccc(NC(=O)[C@H](C)NC(=O)OC(C)(C)C)cc1)C(=O)N(C)[C@H](C[C@@H](OC(C)=O)c1nc(C(=O)Oc2c(F)c(F)c(F)c(F)c2F)cs1)C(C)C]

To a solution of compound 32 (155 mg, 0.433 mmol) in CH₃CN (10 mL) was added 31 (300 mg, 0.433 mmol). The mixture was stirred at 80° C. for 72 h and then concentrated in vacuo to afford 33 (420 mg, crude) as a white solid. ESI MS m/z: calcd for C₄₆H₆₂F₅N₆O₉S [M]⁺: 969.40; found 968.91.

### Example 25: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((S)-2-((tert-butoxycarbonyl)amino)propanamido)benzyl)-N,N,2-trimethyl-1-oxopropan-2-aminium (35)

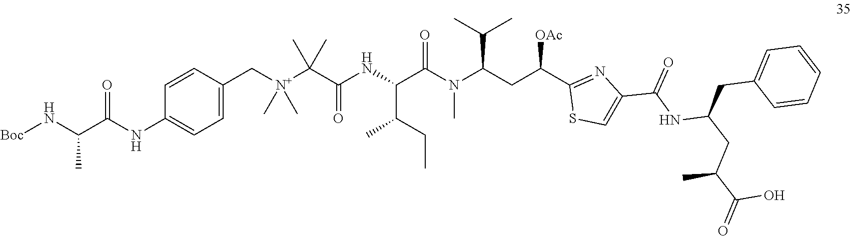 [SMILES: CC[C@H](C)[C@H](NC(=O)C(C)(C)[N+](C)(C)Cc1ccc(NC(=O)[C@H](C)NC(=O)OC(C)(C)C)cc1)C(=O)N(C)[C@H](C[C@@H](OC(C)=O)c1nc(C(=O)N[C@@H](Cc2ccccc2)C[C@H](C)C(=O)O)cs1)C(C)C]

To a solution of compound 33 (78 mg, 0.080 mmol) and compound 34 (17 mg, 0.080 mmol) in DMF (2 mL) was added DIEA (21 mg, 0.161 mmol). The mixture was stirred at RT for 0.5 h, and purified by prep-HPLC to afford 35 (43 mg, 54% yield) as a white solid. ESI MS m/z: calcd for C₅₂H₇₈N₇O₁₀S [M]⁺: 992.60; found 992.08.

### Example 26: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((S)-2-aminopropanamido)benzyl)-N,N,2-trimethyl-1-oxopropan-2-aminium (36)

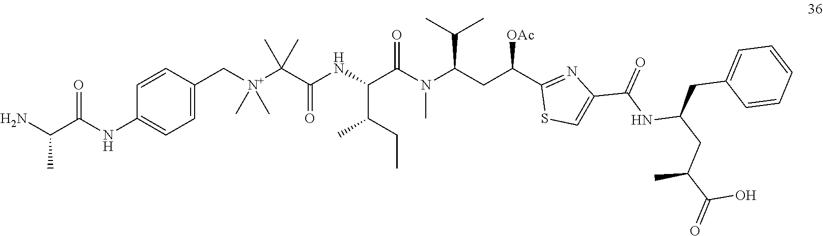 [SMILES: CC[C@H](C)[C@H](NC(=O)C(C)(C)[N+](C)(C)Cc1ccc(NC(=O)[C@H](C)N)cc1)C(=O)N(C)[C@H](C[C@@H](OC(C)=O)c1nc(C(=O)N[C@@H](Cc2ccccc2)C[C@H](C)C(=O)O)cs1)C(C)C]

To a solution of compound 35 (43 mg, 0.043 mmol) in DCM (4 mL) was added TFA (1 mL). The mixture was stirred for 0.5 hours at RT, concentrated in vacuo to afford 36 (38 mg, crude) as a white solid. ESI MS m/z: calcd for C₄₇H₇₀N₇O₈S [M]⁺: 892.51; found 891.92.

### Example 27: perfluorophenyl (tert-butoxycarbonyl)-L-alaninate (37)

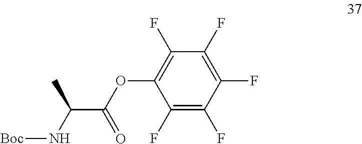 [SMILES: C[C@H](NC(=O)OC(C)(C)C)C(=O)Oc1c(F)c(F)c(F)c(F)c1F]

To a solution of (tert-butoxycarbonyl)-L-alanine (2.00 g, 10.570 mmol) and PFP—OH (1.95 g, 10.570 mmol) in DCM (20 mL) was added EDCI (2.03 g, 10.570 mmol) at room temperature. The mixture was stirred at room temperature for 1.0 h, washed with brine (10 mL×2), then concentrated under reduced pressure to give 37 (3.50 g, 93% yield) as a white solid. ESI MS m/z: calcd for C₁₄H₁₄F₅NO₄ [M+H]⁺: 356.08; found 355.26

### Example 28: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((S)-2-((S)-2-((tert-butoxycarbonyl)amino)propanamido)propanamido)benzyl)-N,N,2-trimethyl-1-oxopropan-2-aminium (38)

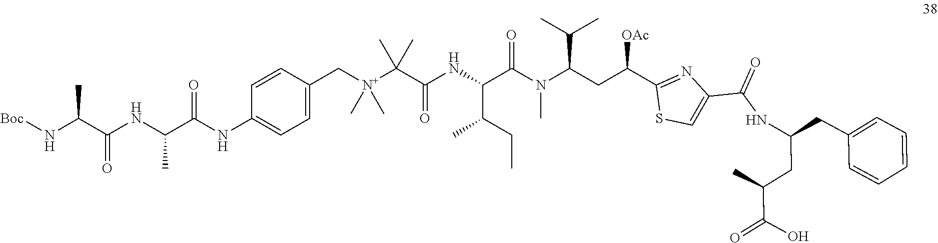 [SMILES: CC[C@H](C)[C@H](NC(=O)C(C)(C)[N+](C)(C)Cc1ccc(NC(=O)[C@H](C)NC(=O)[C@H](C)NC(=O)OC(C)(C)C)cc1)C(=O)N(C)[C@H](C[C@@H](OC(C)=O)c1nc(C(=O)N[C@@H](Cc2ccccc2)C[C@H](C)C(=O)O)cs1)C(C)C]

To a solution of compound 36 (329 mg, 0.369 mmol), compound 37 (170 mg, 0.479 mmol) in DMF (5 mL) was added DIEA (95 mg, 0.736 mmol). The reaction mixture was stirred at room temperature for 1.5 h, and then concentrated under reduced pressure to give 38 (390 mg, 100% yield) as light yellow oil. ESI MS m/z: calcd for C₅₅H₈₃N₈O₁₁S [M]⁺: 1063.59; found 1064.21.

### Example 29: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((S)-2-((S)-2-aminopropanamido)propanamido)benzyl)-N,N,2-trimethyl-1-oxopropan-2-aminium (39)

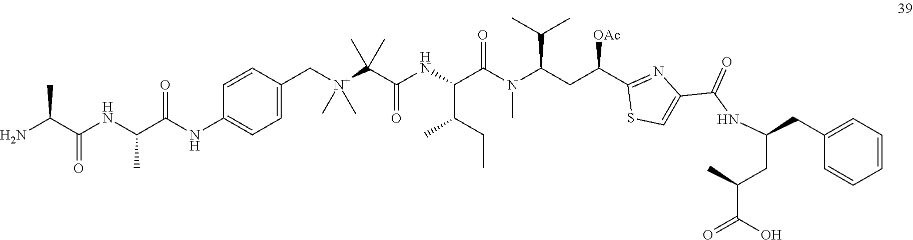

To a solution of compound 38 (390 mg, 0.366 mmol) in DCM (6 mL) was added TFA (3 mL). The mixture was stirred at room temperature for 1 h, and then concentrated under reduced pressure. The resulting residue was purified by prep-HPLC to afford 39 (225 mg, 64% yield) as a white solid. ESI MS m/z: calcd for C₅₀H₇₅N₈O₉ S [M]⁺: 963.54; found 963.75.

### Example 30: N-(4-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (40)

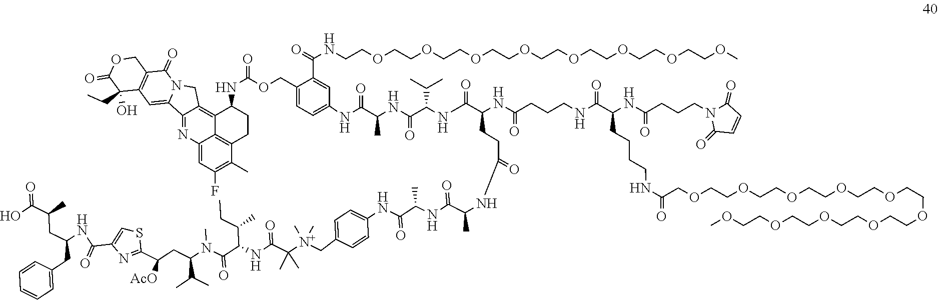 [SMILES: CC[C@H](C)[C@H](NC(=O)C(C)(C)[N+](C)(C)Cc1ccc(NC(=O)[C@H](C)NC(=O)[C@H](C)NC(=O)CC[C@H](NC(=O)CCCNC(=O)[C@H](CCCCNC(=O)COCCOCCOCCOCCOCCOCCOCCOCCOCCOC)NC(=O)CCCN2C(=O)C=CC2=O)C(=O)N[C@H](C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)c(C(=O)NCCOCCOCCOCCOCCOCCOCCOCCOC)c2)C(C)C)cc1)C(=O)N(C)[C@H](C[C@@H](OC(C)=O)c1nc(C(=O)N[C@@H](Cc2ccccc2)C[C@H](C)C(=O)O)cs1)C(C)C]

To a solution of compound 39 (100 mg, 0.043 mmol) and compound 20 (42 mg, 0.043 mmol) in DMF (2 mL) was added DIPEA (44 mg, 0.086 mmol). The mixture was stirred at RT for 1 h and purified by prep-HPLC to afford 40 (77 mg, 59% yield) as a white solid. ESI: m/z: calcd for C₁₅₅H₂₄₄FN₂₀O₄₄ S [M]²⁺: 1542.28; found 1542.28.

### Example 31: methyl (((9H-fluoren-9-yl)methoxy)carbonyl)-L-alaninate-CTC resin (46)

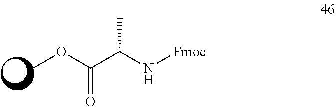

A peptide synthesizer glass vessel (Chemgalss, 100-mL) were charged with 2-chlorotrityl chloride resin (3.30 g, 1.5 g/mmol), 45 (3.08 g, 9.900 mmol) in 50 mL of anhydrous DCM and DIPEA (1.92 g, 14.850 mmol). The reaction was run overnight and the resin was washed with DCM (3×50 mL), followed by DMF (2×50 mL) to give compound 46.

### Example 32: methyl (((9H-fluoren-9-yl)methoxy)carbonyl)-L-alanyl-L-alaninate-CTC resin (47)

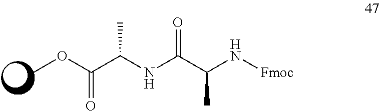

A peptide synthesizer glass vessel (Chemgalss, 100-mL) was charged with compound 46. Fmoc deprotection was performed using 10% piperidine/DMF solution (40 mL). The resin was then washed with DMF (3×50 mL). And then (((9H-fluoren-9-yl)methoxy)carbonyl)-L-alanine (2.31 g, 7.425 mmol) dissolved in 50 mL of anhydrous DMF, HOBt (1.00 g, 7.425 mmol) and DIC (0.94 g, 7.425 mmol) were added. The reaction was run overnight and the resin was washed with DMF (3×50 mL) to give 47.

### Example 33: 5-((S)-2-((S)-2-amino-3-methylbutanamido)propanamido)-2-(hydroxymethyl)-N-(2,5,8,11,14,17,20,23-octaoxapentacosan-25-yl)benzamide-CTC resin (48)

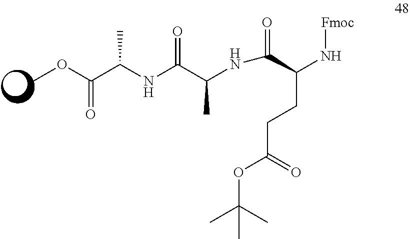

A peptide synthesizer glass vessel (Chemgalss, 100-mL) was charged with compound 47. Fmoc deprotection was performed using 10% piperidine/DMF solution (40 mL). The resin was then washed with DMF (3×50 mL). And then compound 42 (3.16 g, 7.425 mmol) dissolved in 50 mL of anhydrous DMF, HOBt (1.00 g, 7.425 mmol), and DIC (0.94 g, 7.425 mmol) were added. The reaction was run overnight and the resin was washed with DMF (3×50 mL) to give compound 48.

### Example 34: ((S)-5-(tert-butoxy)-2-((S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-amido)-5-oxopentanoyl)-L-alanyl-L-alanine-CTC resin (49)

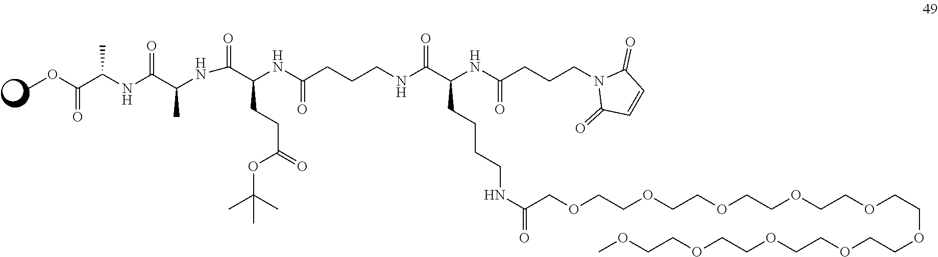 [SMILES: COCCOCCOCCOCCOCCOCCOCCOCCOCCOCC(=O)NCCCC[C@H](NC(=O)CCCN1C(=O)C=CC1=O)C(=O)NCCCC(=O)N[C@@H](CCC(=O)OC(C)(C)C)C(=O)N[C@@H](C)C(=O)N[C@@H](C)C(=O)OC]

A peptide synthesizer glass vessel (Chemgalss, 100-mL) was charged with compound 48. Fmoc deprotection was performed using 10% piperidine/DMF solution (40 mL). The resin was then washed with DMF (3×50 mL). And then compound 12 (5.14 g, 5.940 mmol) dissolved in 50 mL of anhydrous DMF, HOBt (0.80 g, 5.940 mmol), and DIC (0.75 g, 5.940 mmol) were added. The reaction was run overnight and the resin was washed with DMF (3×50 mL) to give compound 49.

### Example 35: ((S)-5-(tert-butoxy)-2-((S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-amido)-5-oxopentanoyl)-L-alanyl-L-alanine (50)

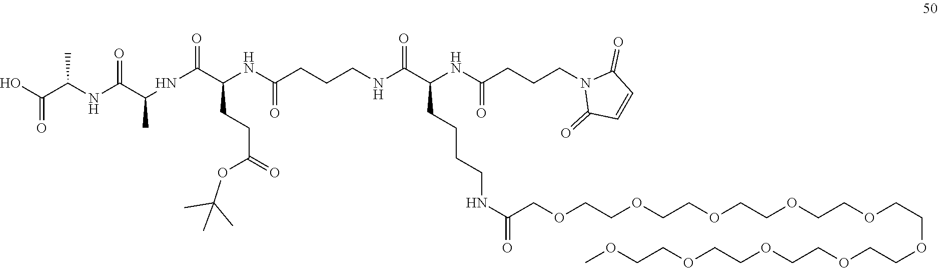 [SMILES: COCCOCCOCCOCCOCCOCCOCCOCCOCCOCC(=O)NCCCC[C@H](NC(=O)CCCN1C(=O)C=CC1=O)C(=O)NCCCC(=O)N[C@@H](CCC(=O)OC(C)(C)C)C(=O)N[C@@H](C)C(=O)N[C@@H](C)C(=O)O]

A peptide synthesizer glass vessel (Chemgalss, 100-mL) was charged with compound 49. The resin cleavage was performed using 1,1,1,3,3,3-hexafluoropropan-2-ol (15 mL) and DCM (35 mL), followed by a DCM wash (3×50 mL). The deprotection eluent was evaporated under reduced pressure to give 50 (5.90 g, 100% yield) as a white oil. ESI MS m/z: calcd for C₅₄H₉₃N₇O₂₂ [M+H]⁺: 1192.64; found 1192.73.

### Example 36: tert-butyl (37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (51)

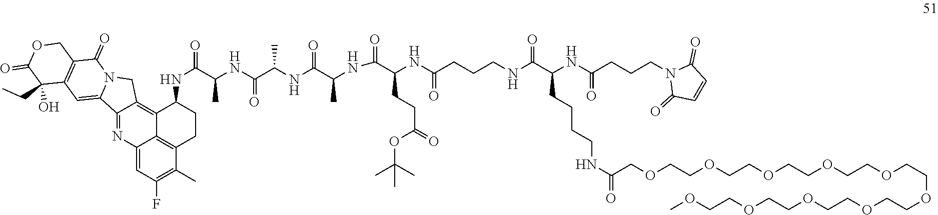 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)NC(=O)[C@H](CCC(=O)OC(C)(C)C)NC(=O)CCCNC(=O)[C@H](CCCCNC(=O)COCCOCCOCCOCCOCCOCCOCCOCCOCCOC)NC(=O)CCCN1C(=O)C=CC1=O)CC3]

To a solution of compound 50 (2.47 g, 2.073 mmol), Exatecan (1.00 g, 1.974 mmolq) and HATU (0.79 g, 2.073 mmol) in DMF (30 mL) was added DIEA (0.51 g, 3.948 mmol).

The reaction mixture was stirred at room temperature for 1.5 h, and then concentrated under reduced pressure to give 51 (3.32 g, 100% yield) as a yellow oil. ESI MS m/z: calcd for C₈₁H₁₁₈FN₁₁O₂₆ [M+H]⁺: 1680.82; found 1681.07.

### Example 37: (37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oic acid (52)

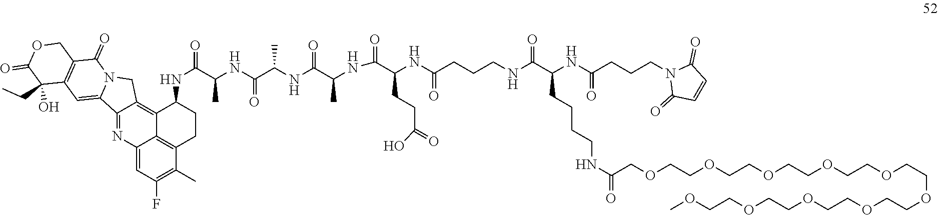 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)NC(=O)[C@H](CCC(=O)O)NC(=O)CCCNC(=O)[C@H](CCCCNC(=O)COCCOCCOCCOCCOCCOCCOCCOCCOCCOC)NC(=O)CCCN1C(=O)C=CC1=O)CC3]

To a solution of compound 51 (3.32 g, 1.975 mmol) in DCM (10 mL) was added TFA (8 mL). The mixture was stirred at room temperature for 1 h, and then concentrated under reduced pressure. The resulting residue was purified by prep-HPLC to give 52 (1.37 g, 42% yield) as a yellow solid. ESI MS m/z: calcd for C₇₇H₁₁₀FN₁₁O₂₆ [M+H]⁺: 1624.76; found 1623.78.

### Example 38: perfluorophenyl (37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (53)

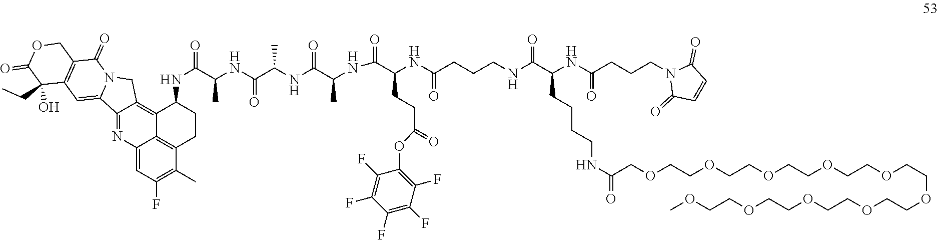 [SMILES: CC[C@@]1(O)C(=O)OCc2c1cc1n(c2=O)Cc2c-1nc1cc(F)c(C)c3c1c2[C@@H](NC(=O)[C@H](C)NC(=O)[C@H](C)NC(=O)[C@H](C)NC(=O)[C@H](CCC(=O)Oc1c(F)c(F)c(F)c(F)c1F)NC(=O)CCCNC(=O)[C@H](CCCCNC(=O)COCCOCCOCCOCCOCCOCCOCCOCCOCCOC)NC(=O)CCCN1C(=O)C=CC1=O)CC3]

To a solution of compound 52 (200 mg, 0.123 mmol) and PFP—OH (27 mg, 0.148 mmol) in DCM (3 mL) was added EDCI (35 mg, 0.185 mmol) at room temperature. The mixture was stirred at room temperature for 1.0 h, washed with brine (10 mL×2), then concentrated under reduced pressure to give 53 (220 mg, 100% yield) as a yellow solid. ESI MS m/z: calcd for C₈₃H₁₀₉F6N₁₁O₂₆ [M+H]⁺: 1790.74 found 1789.86.

### Example 39: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((37S,45S,50S,53S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50,53-dimethyl-31,38,43,48,51-pentaoxo-2,5,8,11,14,17,20,23,26,29-decaoxa-32,39,44,49,52-pentaazatetrapentacontan-54-amido)benzyl)-N,N,2-trimethyl-1-oxopropan-2-aminium (54)

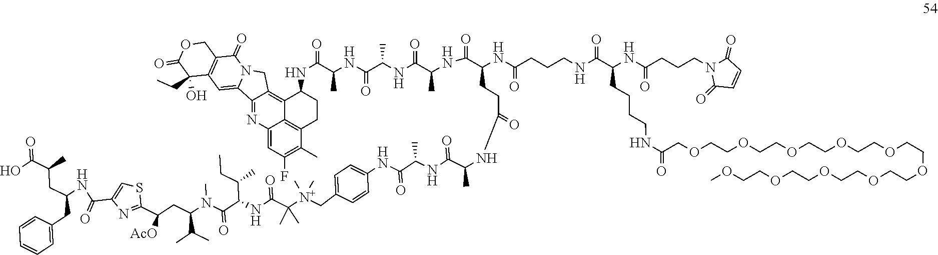 [SMILES: CC[C@H](C)[C@H](NC(=O)C(C)(C)[N+](C)(C)Cc1ccc(NC(=O)[C@H](C)NC(=O)[C@H](C)NC(=O)CC[C@H](NC(=O)CCCNC(=O)[C@H](CCCCNC(=O)COCCOCCOCCOCCOCCOCCOCCOCCOCCOC)NC(=O)CCCN2C(=O)C=CC2=O)C(=O)N[C@@H](C)C(=O)N[C@@H](C)C(=O)N[C@@H](C)C(=O)N[C@H]2CCc3c(C)c(F)cc4nc5c(c2c34)Cn2c-5cc3c(c2=O)COC(=O)[C@]3(O)CC)cc1)C(=O)N(C)[C@H](C[C@@H](OC(C)=O)c1nc(C(=O)N[C@@H](Cc2ccccc2)C[C@H](C)C(=O)O)cs1)C(C)C]

To a solution of compound 53 (80 mg, 0.045 mmol) and compound 39 (43 mg, 0.045 mmol in DMF (2 mL) was added DIEA (12 mg, 0.080 mmol). The mixture was stirred at RT for 1 h, purified by prep-HPLC to afford 54 (63 mg, 52% yield) as a white solid. ESI: m/z: calcd for C₁₂₇H₁₈₃FN₁₉O₃₄S [M+H]⁺: 2570.29; found 2570.29.

### Example 40: tert-butyl (2S,4R)-5-(3-((S)-2-((((9H-fluoren-9-yl)methoxy)carbonyl)amino)propanamido)-4-hydroxyphenyl)-4-((tert-butoxycarbonyl)amino)-2-methylpentanoate (59)

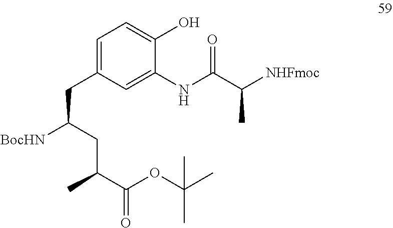

To a solution of compound 58 (0.89 g, 2.260 mmol) in THF (20 mL) at room temperature were added Fmoc-L-Alanine (0.70 g, 2.260 mmol), HATU (0.94 g, 2.480 mmol) and DIEA (0.58 g, 4.510 mmol). The reaction mixture was stirred at room temperature for 1 h, diluted with EA (200 mL), then washed with 5% Na₂CO₃ (50 mL), 1 N HCl (50 mL), dried over Na₂SO₄, filtered and concentrated under reduced pressure. The resulting residue was purified by silica gel column, eluted with ethyl acetate and petroleum ether, concentrated to afford 59 (1.40 g, 90% yield) as a white solid. ESI: m/z: calcd for C₃₉H₄₉N₃O₈ [M+H]⁺: 688.35; found 688.41.

### Example 41: tert-butyl (2S,4R)-5-(3-((S)-2-aminopropanamido)-4-hydroxyphenyl)-4-((tert-butoxycarbonyl)amino)-2-methylpentanoate (60)

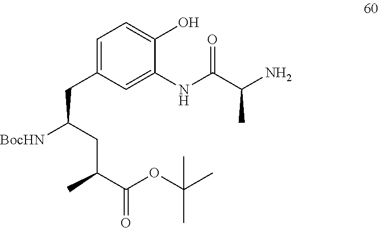 [SMILES: C[C@H](N)C(=O)Nc1cc(C[C@@H](C[C@H](C)C(=O)OC(C)(C)C)NC(=O)OC(C)(C)C)ccc1O]

To a solution of compound 59 (1.40 g, 2.040 mmol) in DCM (20 mL) at room temperature was added diethylamine (10 mL). The reaction mixture was stirred at room temperature for 1 h, concentrated to afford 60 (0.95 g, 100% yield) as a yellow oil. ESI: m/z: calcd for C₂₄H₃₉N₃O₆ [M+H]⁺: 466.28; found 466.35.

### Example 42: tert-butyl (2S,4R)-5-(3-((S)-2-((S)-2-((((9H-fluoren-9-yl)methoxy)carbonyl)amino)-3-methylbutanamido)propanamido)-4-hydroxyphenyl)-4-((tert-butoxycarbonyl)amino)-2-methylpentanoate (61)

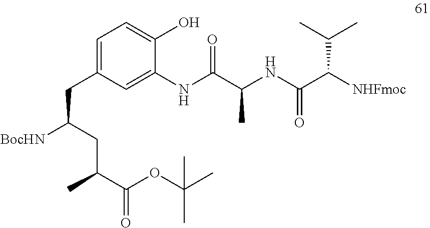

To a solution of compound 60 (0.95 g, 2.040 mmol) in THF (20 mL) at room temperature were added Fmoc-L-Valine (0.69 g, 2.040 mmol), HATU (1.01 g, 2.650 mmol) and DIEA (0.53 g, 4.080 mmol). The reaction mixture was stirred at room temperature for 0.5 h and diluted with EA (150 mL), washed with 5% Na₂CO₃ (50 mL), 1 N HCl (50 mL), dried over Na₂SO₄, filtered and concentrated under reduced pressure. The resulting residue was purified by silica gel column, eluted with ethyl acetate and petroleum ether, concentrated to afford 61 (1.00 g, 67% yield) as a white solid. ESI: m/z: calcd for C₄₄H₅₈N₄O₉ [M+H]⁺: 787.42; found 787.36.

### Example 43: (2S,4R)-5-(3-((S)-2-((S)-2-((((9H-fluoren-9-yl)methoxy)carbonyl)amino)-3-methylbutanamido)propanamido)-4-hydroxyphenyl)-4-amino-2-methylpentanoic acid (62)

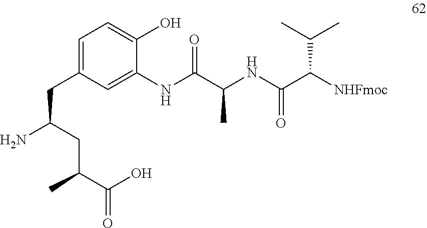

To a solution of compound 61 (0.90 g, 1.140 mmol) in DCM (5 mL) at room temperature was added TFA (5 mL). The reaction mixture was stirred at room temperature for 1.5 h, concentrated to afford 62 (0.72 g, 100% yield) as a yellow oil. ESI: m/z: calcd for C₃₅H₄₂N₄O₇ [M+H]⁺: 631.31; found 631.58.

### Example 44: (2S,4R)-5-(3-((S)-2-((S)-2-((((9H-fluoren-9-yl)methoxy)carbonyl)amino)-3-methylbutanamido)propanamido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (63)

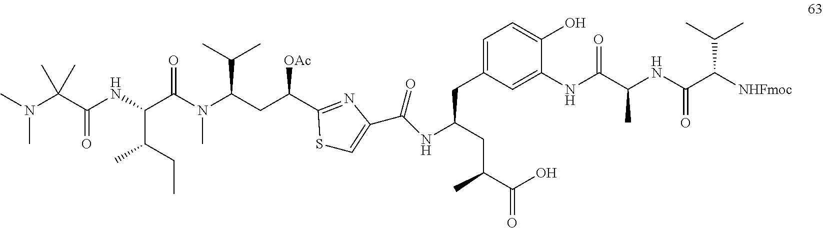

To a solution of compound 62 (0.72 g, 1.140 mmol) in DCM (30 mL) at room temperature were added compound 31 (0.79 g, 1.140 mmol) and DIEA (0.44 g, 3.430 mmol, 2.00 eq). The reaction mixture was stirred at room temperature overnight, purified by silica gel column, eluted with DCM and methanol, concentrated to afford 63 (1.30 g, 100% yield) as a white solid. ESI: m/z: calcd for C₆₀H₈₂N₈O₁₂S [M+H]⁺: 1139.58; found 1139.21.

### Example 45: (2S,4R)-5-(3-((S)-2-((S)-2-amino-3-methylbutanamido)propanamido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (64)

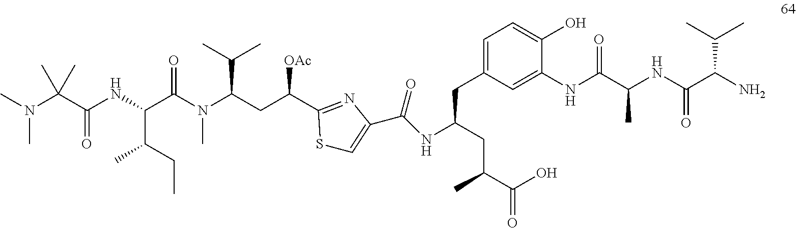 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(O)c(NC(=O)[C@H](C)NC(=O)[C@@H](N)C(C)C)c2)C[C@H](C)C(=O)O)cs1)C(C)C]

To a solution of compound 63 (1.30 g, 1.140 mmol) in DCM (10 mL) at room temperature was added diethylamine (5 mL). The reaction mixture was stirred at room temperature for 0.5 h, concentrated to afford 64 (0.46 g, 44% yield) as a white solid. ESI: m/z: calcd for C₄₅H₇₂N₈O₁₀S [M+H]⁺: 917.51; found 917.25.

### Example 46: (2S,4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(3-((37S,45S,50S,53S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51-pentaoxo-2,5,8,11,14,17,20,23,26,29-decaoxa-32,39,44,49,52-pentaazatetrapentacontan-54-amido)-4-hydroxyphenyl)-2-methylpentanoic acid (65)

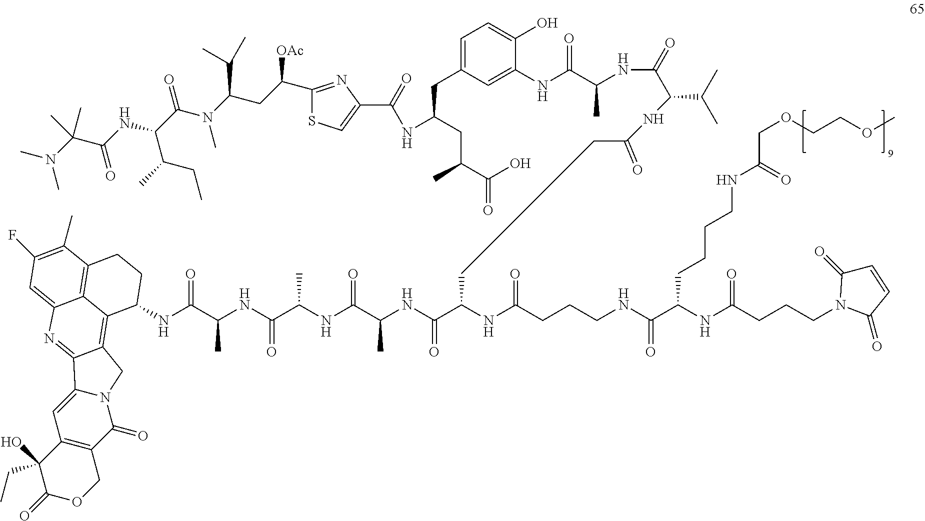

To a solution of compound 63 (51 mg, 0.060 mmol) in DMF (2 mL) at room temperature were added compound 53 (100 mg, 0.060 mmol) and DIEA (14 mg, 0.110 mmol). The reaction mixture was stirred at room temperature for 2 h, purified by prep-HPLC, lyophilized to afford 65 (68 mg, 49% yield) as a white solid. ESI: m/z: calcd for C₁₂₂H₁₈₀FN₁₉O₃₅S [M+2H]²⁺: 1262.13; found 1262.08.

### Example 47: tert-butyl (2S,4R)-5-(3-(4-((((9H-fluoren-9-yl)methoxy)carbonyl)amino) butanamido)-4-hydroxyphenyl)-4-((tert-butoxycarbonyl)amino)-2-methylpentanoate (68)

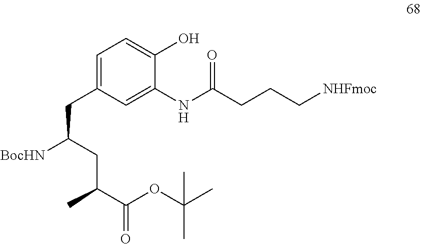

To a solution of compound 67 (1.40 g, 3.550 mmol) in DCM (100 mL) at room temperature were added Fmoc-GABA-OH (1.27 g, 3.900 mmol), HATU (1.75 g, 4.610 mmol) and DIEA (0.92 g, 7.100 mmol). The reaction mixture was stirred at room temperature for 1 h. The mixture was washed with 1 N HCl (50 mL), dried over Na₂SO₄, filtered and concentrated under reduced pressure. The resulting residue was purified by silica gel column, eluted with ethyl acetate and petroleum ether, concentrated to afford 68 (1.70 g, 68% yield) as a white solid. ESI: m/z: calcd for C₄₀H₅₁N₃O₈ [M+H]⁺: 702.37; found 702.54.

### Example 48: tert-butyl (2S,4R)-5-(3-(4-aminobutanamido)-4-hydroxyphenyl)-4-((tert-butoxycarbonyl)amino)-2-methylpentanoate (69)

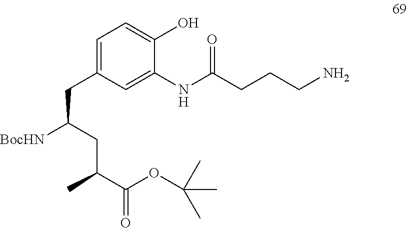 [SMILES: C[C@@H](C[C@H](Cc1ccc(O)c(NC(=O)CCCN)c1)NC(=O)OC(C)(C)C)C(=O)OC(C)(C)C]

To a solution of compound 68 (1.70 g, 2.410 mmol) in DCM (10 mL) at room temperature was added diethylamine (10 mL). The reaction mixture was stirred at room temperature for 2 h, concentrated to afford 69 (1.16 g, 100% yield) as a yellow oil. ESI: m/z: calcd for C₂₅H₄₁N₃O₆ [M+H]⁺: 480.30; found 480.21.

### Example 49: tert-butyl (2S,4R)-5-(3-((5S,8S)-1-(9H-fluoren-9-yl)-5-isopropyl-8-methyl-3,6,9-trioxo-2-oxa-4,7,10-triazatetradecan-14-amido)-4-hydroxyphenyl)-4-((tert-butoxycarbonyl)amino)-2-methylpentanoate (71)

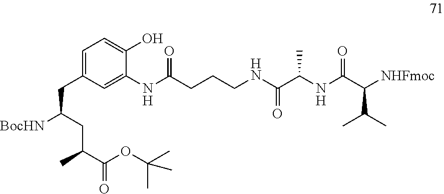

To a solution of compound 69 (1.16 g, 2.410 mmol) in DCM (30 mL) at room temperature were added compound 70 (0.99 g, 2.410 mmol), HATU (1.10 g, 2.900 mmol) and DIEA (0.63 g, 4.820 mmol). The reaction mixture was stirred at room temperature for 0.5 h. The mixture was washed with 1 N HCl (50 mL), dried over Na₂SO₄, filtered and concentrated under reduced pressure. The resulting residue was purified by silica gel column, eluted with ethyl acetate and petroleum ether, concentrated to afford 71 (2.11 g, 100% yield) as a white solid. ESI: m/z: calcd for C₄₈H₆₅N₅O₁₀ [M+H]⁺: 872.47; found 872.54.

### Example 50: (2S,4R)-5-(3-((5S,8S)-1-(9H-fluoren-9-yl)-5-isopropyl-8-methyl-3,6,9-trioxo-2-oxa-4,7,10-triazatetradecan-14-amido)-4-hydroxyphenyl)-4-amino-2-methylpentanoic acid (72)

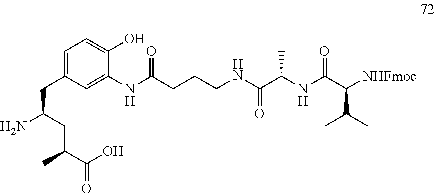

To a solution of compound 71 (1.00 g, 1.140 mmol) in DCM (5 mL) at room temperature was added TFA (10 mL). The reaction mixture was stirred at room temperature for 1 h, concentrated to afford 72 (0.82 g, 100% yield) as a yellow oil. ESI: m/z: calcd for C₃₉H₄₉N₅O₈ [M+H]⁺: 716.36; found 716.26.

### Example 51: (2S,4R)-5-(3-((5S,8S)-1-(9H-fluoren-9-yl)-5-isopropyl-8-methyl-3,6,9-trioxo-2-oxa-4,7,10-triazatetradecan-14-amido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (73)

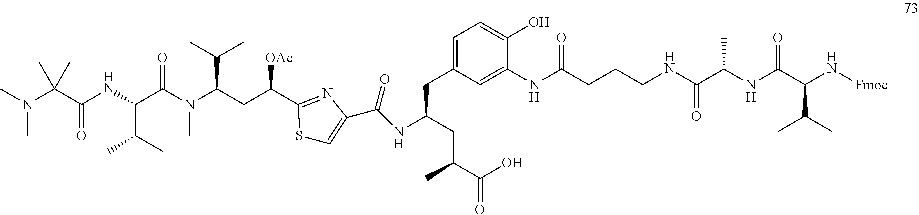

To a solution of compound 72 (0.82 g, 1.140 mmol) in DCM (20 mL) at room temperature were added compound 31 (0.79 g, 1.140 mmol) and DIEA (0.59 g, 3.580 mmol). The reaction mixture was stirred at room temperature overnight, purified by silica gel column, eluted with DCM and methanol, concentrated to afford 73 (1.40 g, 100% yield) as a yellow oil. ESI: m/z: calcd for C₆₄H₈₉N₉O₁₃S [M+H]⁺: 1224.63; found 1225.01

### Example 52: (2S,4R)-5-(3-(4-((S)-2-((S)-2-amino-3-methylbutanamido)propanamido) butanamido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (74)

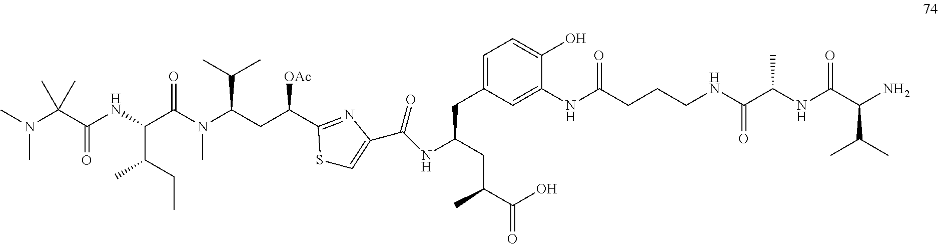 [SMILES: CC[C@H](C)[C@H](NC(=O)C(C)(C)N(C)C)C(=O)N(C)[C@H](C[C@@H](OC(C)=O)c1nc(C(=O)N[C@@H](Cc2ccc(O)c(NC(=O)CCCNC(=O)[C@H](C)NC(=O)[C@@H](N)C(C)C)c2)C[C@H](C)C(=O)O)cs1)C(C)C]

To a solution of compound 73 (1.40 g, 1.140 mmol) in DCM (10 mL) at room temperature was added diethylamine (10 mL). The reaction mixture was stirred at room temperature for 0.5 h, concentrated to afford 74 (0.50 g, 43% yield) as a yellow oil. ESI: m/z: calcd for C₄₉H₇₉N₉O₁₁S [M+H]⁺: 1002.56; found 1002.89.

### Example 53: (2S,4R)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-5-(3-((37S,45S,50S,53S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51,54-hexaoxo-2,5,8,11,14,17,20,23,26,29-decaoxa-32,39,44,49,52,55-hexaazanonapentacontan-59-amido)-4-hydroxyphenyl)-2-methylpentanoic acid (75)

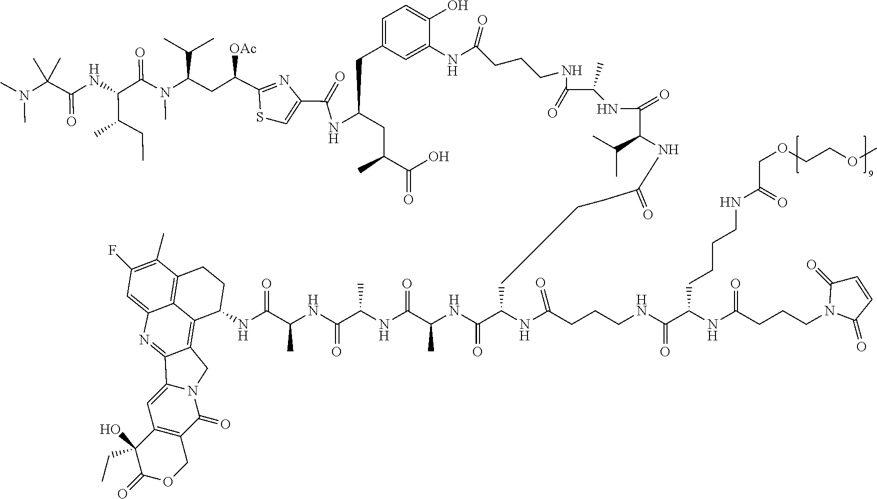

To a solution of compound 74 (55 mg, 0.060 mmol) in DMF (2 mL) at room temperature were added compound 53 (99 mg, 0.060 mmol) and DIEA (14 mg, 0.12 mmol). The reaction mixture was stirred at room temperature for 1 h, purified by prep-HPLC, lyophilized to afford 75 (15 mg, 11% yield) as a white solid. ESI: m/z: calcd for C₁₂₆H₁₈₇FN₂₀O₃₆S [M+2H]²⁺: 1304.66; found 1304.53.

### Example 54: 2,3-bis(((benzyloxy)carbonyl)amino)-4-((4-(tert-butoxy)-4-oxobutyl)amino)-4-oxobutanoic acid (78)

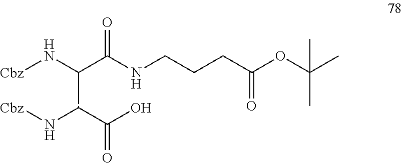 [SMILES: CC(C)(C)OC(=O)CCCNC(=O)C(CC(NC(=O)OCc1ccccc1)C(=O)O)NC(=O)OCc1ccccc1]

To a solution of compound 77 (2.89 g, 6.900 mmol) in DMF (60 mL) at room temperature were added 2 (1.36 g, 6.900 mmol) and DMTMM (3.07 g, 10.400 mmol). The reaction mixture was stirred at room temperature for 2 days. The reaction mixture was resolved in EA (300 mL), then washed with 1 N HCl (100 mL), brine (100 mL), dried over Na₂SO₄, filtered and concentrated under reduced pressure, purified by silica gel column, eluted with DCM and methanol, concentrated to afford 78 (2.30 g, 59% yield) as a white solid. ESI: m/z: calcd for C₂₈H₃₅N₃O₉ [M+H]⁺: 558.24; found 558.32.

### Example 55: tert-butyl 4-(3,4-bis(((benzyloxy)carbonyl)amino)-2,5-dioxopyrrolidin-1-yl) butanoate (79)

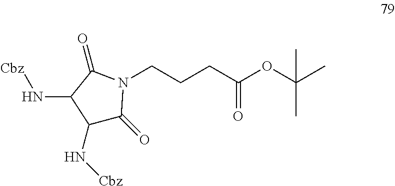 [SMILES: CC(C)(C)OC(=O)CCCN1C(=O)C(NC(=O)OCc2ccccc2)C(NC(=O)OCc2ccccc2)C1=O]

To a solution of compound 78 (2.10 g, 3.800 mmol) in DMF (30 mL) at room temperature were added HATU (2.86 g, 7.500 mmol) and TEA (0.76 g, 7.500 mmol). The reaction mixture was stirred at room temperature for 1 day. The reaction mixture was diluted with EA (300 mL), then washed with 1 N HCl (100 mL), brine (100 mL), dried over Na₂SO₄, filtered and concentrated under reduced pressure, purified by silica gel column, eluted with DCM and methanol, concentrated to afford 79 (1.40 g, 69% yield) as a white solid. ESI: m/z: calcd for C₂₈H₃₃N₃O₈ [M+H]⁺: 540.23; found 540.12.

### Example 56: tert-butyl 4-(3,4-diamino-2,5-dioxopyrrolidin-1-yl) butanoate (80)

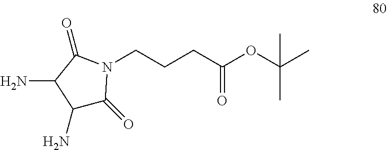 [SMILES: CC(C)(C)OC(=O)CCCN1C(=O)C(N)C(N)C1=O]

To a solution of compound 79 (1.40 g, 5.000 mmol) in isopropanol (40 mL) at 70° C. was added 10% Pd/C (0.60 g). The mixture was stirred overnight under hydrogen balloon at 70° C. The mixture was filtered with Celite. The filtrated was concentrated to give 80 (0.80 g, 63% yield) as a brown oil. ESI: m/z: calcd for C₁₂H₂₁N₃O₄ [M+H]⁺: 272.15; found 272.11.

### Example 57: 3,3′-(((1-(4-(tert-butoxy)-4-oxobutyl)-2,5-dioxopyrrolidine-3,4-diyl)bis(azanediyl))bis(carbonyl))bis(7-oxabicyclo[2.2.1]hept-5-ene-2-carboxylic acid) (81)

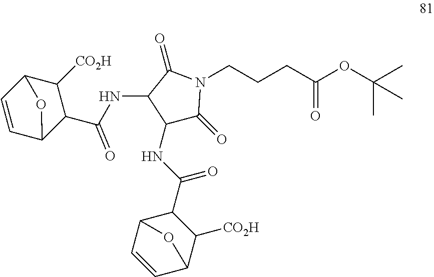 [SMILES: CC(C)(C)OC(=O)CCCN1C(=O)C(NC(=O)C2C3C=CC(O3)C2C(=O)O)C(NC(=O)C2C3C=CC(O3)C2C(=O)O)C1=O]

To a solution of compound 80 (0.80 g, 2.900 mmol) in DCM (20 mL) at room temperature were added exo-3,6-Epoxy-1,2,3,6-tetrahydrophthalic Anhydride (0.98 g, 5.900 mmol) and TEA (0.60 g, 5.900 mmol). The reaction mixture was stirred at room temperature for 1 h, and then concentrated to give 81 (1.78 g, 100% yield) as a white solid. ESI: m/z: calcd for C₂₈H₃₃N₃O₁₂ [M+H]⁺: 604.21; found 604.58.

### Example 58: tert-butyl 4-(3,4-bis(1,3-dioxo-1,3,3a,4,7,7a-hexahydro-2H-4,7-epoxyisoindol-2-yl)-2,5-dioxopyrrolidin-1-yl) butanoate (82)

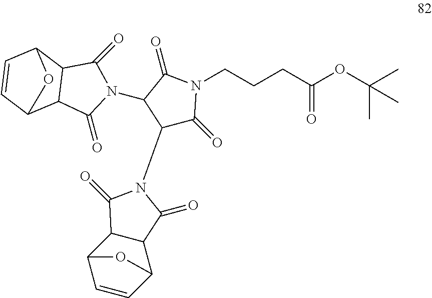 [SMILES: CC(C)(C)OC(=O)CCCN1C(=O)C(N2C(=O)C3C4C=CC(O4)C3C2=O)C(N2C(=O)C3C4C=CC(O4)C3C2=O)C1=O]

To a solution of compound 81 (1.76 g, 2.900 mmol) in DMF (20 mL) at room temperature were added EDCI (1.68 g, 8.700 mmol), HOBt (1.18 g, 8.700 mmol) and DBU (1.33 g, 8.700 mmol). The reaction mixture was stirred at 60° C. for 4 h. The reaction mixture was washed with water (100 mL), NaHCO₃ solution (100 mL×2), water (100 mL), 1 N HCl (100 mL), brine (100 mL), dried over Na₂SO₄, filtered and concentrated under reduced pressure, purified by prep-HPLC, lyophilized to afford 82 (1.30 g, 79% yield) as a white solid. ESI: m/z: calcd for C₂₈H₂₉N₃O₁₀ [M+H]⁺: 568.19; found 568.19.

### Example 59: tert-butyl 4-(3,4-bis(2,5-dioxo-2,5-dihydro-1H-pyrrol-1-yl)-2,5-dioxopyrrolidin-1-yl) butanoate (83)

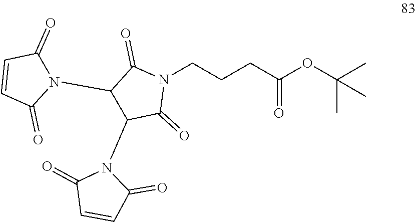 [SMILES: CC(C)(C)OC(=O)CCCN1C(=O)C(N2C(=O)C=CC2=O)C(N2C(=O)C=CC2=O)C1=O]

A solution of compound 82 (1.30 g, 2.300 mmol) in toluene (20 mL) was stirred at 120° C. for 6 h, and then concentrated to give 83 (0.90 g, 91% yield) as a white solid. ESI: m/z: calcd for C₂₀H₂₁N₃O₈ [M+H]⁺: 432.13; found 432.14.

### Example 60:4-(3,4-bis(2,5-dioxo-2,5-dihydro-1H-pyrrol-1-yl)-2,5-dioxopyrrolidin-1-yl) butanoic acid (84)

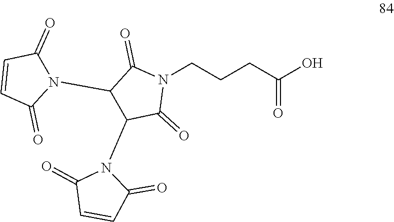 [SMILES: O=C(O)CCCN1C(=O)C(N2C(=O)C=CC2=O)C(N2C(=O)C=CC2=O)C1=O]

To a solution of compound 83 (0.90 g, 2.000 mmol) in CH₂Cl₂ (10 mL) at room temperature was added TFA (5 mL). The reaction mixture was stirred at room temperature for 1 h, concentrated to afford 84 (0.83 g, 100% yield) as a yellow oil. ESI: m/z: calcd for C₁₆H₁₃N₃O₈ [M+H]⁺: 376.07; found 376.57.

### Example 61:4-(3,4-bis(2,5-dioxo-2,5-dihydro-1H-pyrrol-1-yl)-2,5-dioxopyrrolidin-1-yl) butanoic acid (85)

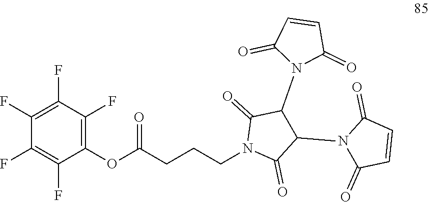 [SMILES: O=C(CCCN1C(=O)C(N2C(=O)C=CC2=O)C(N2C(=O)C=CC2=O)C1=O)Oc1c(F)c(F)c(F)c(F)c1F]

To a solution of compound 84 (0.20 g, 0.500 mmol) in DCM (15 mL) at room temperature were added EDCI (0.20 g, 1.100 mmol), PFP (0.11 g, 0.600 mmol). The reaction mixture was stirred at room temperature for 1 h, then washed with brine (20 mL×2), dried over Na₂SO₄, filtered and concentrated under reduced pressure to afford 85 (0.29 g, 100% yield) as a white solid. ESI: m/z: calcd for C₂₂H₁₂F5N₃O₈ [M+H]⁺: 542.05; found 542.15.

### Example 62: perfluorophenyl (37S,45S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (87)

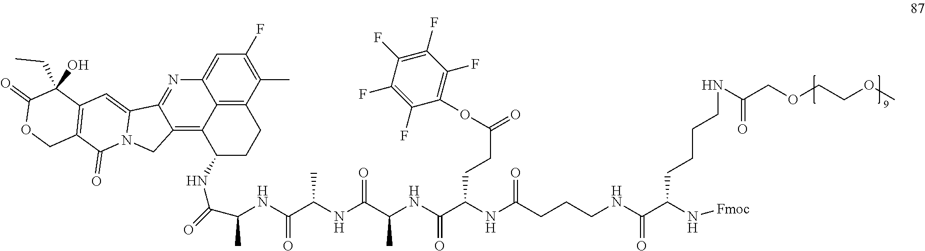

To a solution of compound 86 (0.25 g, 0.150 mmol) in DCM (10 mL) at room temperature were added EDCI (57 mg, 0.180 mmol), PFP—OH (57 mg, 0.300 mmol). The reaction mixture was stirred at room temperature for 2 h, then washed with brine (20 mL×2), dried over Na₂SO₄, filtered and concentrated under reduced pressure to afford 87 (0.27 g, 100% yield) as a yellow solid. ESI: m/z: calcd for C₉₀H₁₁₂F6N₁₀O₂₅ [M+H]⁺: 1847.77; found 1847.25.

### Example 63: (2S,4R)-5-(3-((37S,45S,50S,53S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51-pentaoxo-2,5,8,11,14,17,20,23,26,29-decaoxa-32,39,44,49,52-pentaazatetrapentacontan-54-amido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (88)

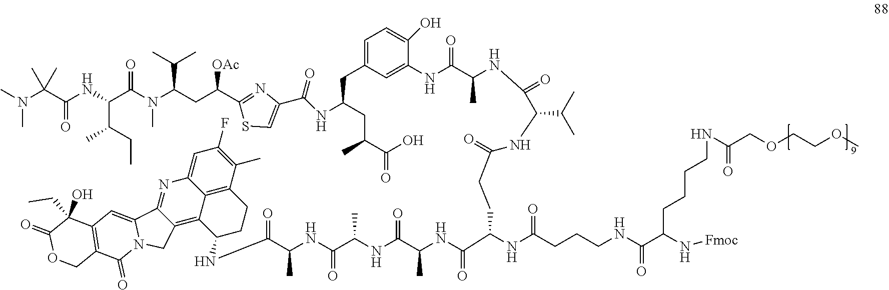

To a solution of compound 74 (124 mg, 0.140 mmol) in DMF (2 mL) at room temperature were added compound 87 (250 mg, 0.140 mmol) and DIEA (35 mg, 0.270 mmol). The reaction mixture was stirred at room temperature for 1 h, purified by prep-HPLC, lyophilized to afford 88 (350 mg, 100% yield) as a white solid. ESI: m/z: calcd for C₁₂₉H₁₈₃FN₁₈O₃₄S [M+2H]²⁺: 1290.64; found 1290.86.

### Example 64: (2S,4R)-5-(3-((37S,45S,50S,53S)-37-amino-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51-pentaoxo-2,5,8,11,14,17,20,23,26,29-decaoxa-32,39,44,49,52-pentaazatetrapentacontan-54-amido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (89)

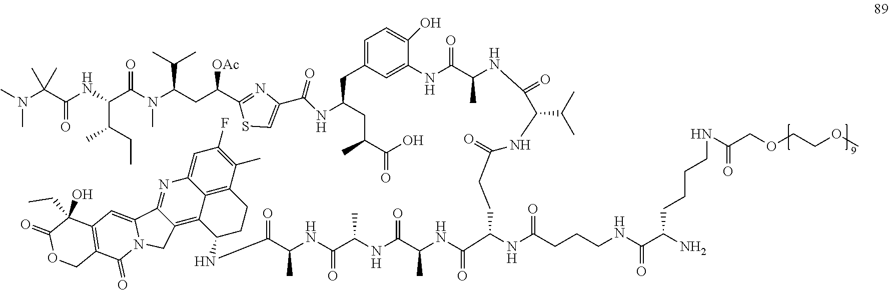

To a solution of compound 88 (350 mg, 0.140 mmol) in DCM (10 mL) at room temperature was added diethylamine (5 mL). The reaction mixture was stirred at room temperature for 2 h, concentrated, purified by prep-HPLC, lyophilized to afford 89 (140 mg, 44% yield) as a white solid. ESI: m/z: calcd for C₁₁₄H₁₇₃FN₁₈O₃₂S [M+H]⁺: 2358.22; found 2358.58.

### Example 65: (2S,4R)-5-(3-((37S,45S,50S,53S)-37-(4-(3,4-bis(2,5-dioxo-2,5-dihydro-1H-pyrrol-1-yl)-2,5-dioxopyrrolidin-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51-pentaoxo-2,5,8,11,14,17,20,23,26,29-decaoxa-32,39,44,49,52-pentaazatetrapentacontan-54-amido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (90)

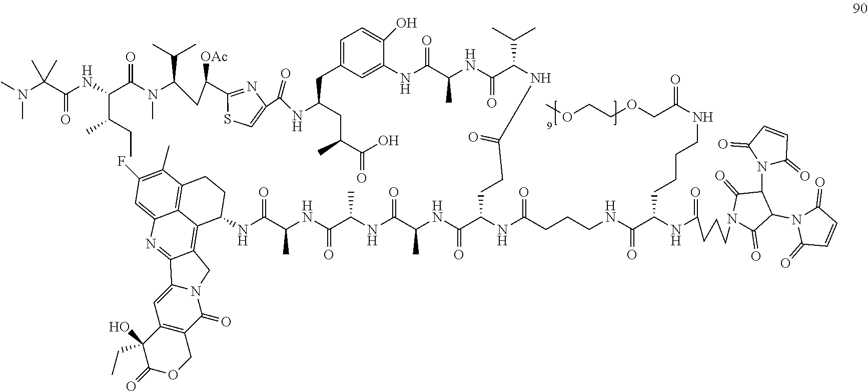

To a stirring solution of compound 89 (90 mg, 0.040 mmol) in DMF (2 mL) at room temperature were added compound 85 (21 mg, 0.040 mmol) and DIEA (10 mg, 0.070 mmol). The reaction mixture was stirred at room temperature for 0.5 h, purified by prep-HPLC, lyophilized to afford 90 (25 mg, 24% yield). ESI MS m/z: calcd for C₁₃₀H₁₈₄FN₂₁O₃₉S [M+2H]²⁺: 1358.14; found 1358.24.

### Example 66:2,5,8,11,14,17,20,23,26,29-decaoxahentriacontan-31-oyl chloride (92)

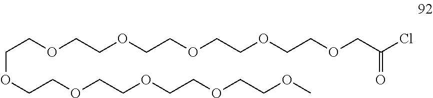 [SMILES: COCCOCCOCCOCCOCCOCCOCCOCCOCCOCC(=O)Cl]

To a solution of compound 91 (22.00 g, 45.215 mmol) in DCM (220 mL) were added oxalyl chloride (11.48 g, 90.431 mmol) and DMF (0.2 mL). The mixture was stirred at room temperature for 3 h, and then concentrated under reduced pressure to give 92 (22.83 g, 100% yield) as a yellow oil. ESI MS m/z: calcd for C₂₁H₄₁ClO₁₁ [M+H]⁺: 505.23 found 504.27.

### Example 67: (S)-37-((((9H-fluoren-9-yl)methoxy)carbonyl)amino)-31-oxo-2,5,8,11,14,17,20,23,26,29-decaoxa-32-azaoctatriacontan-38-oic acid (94)

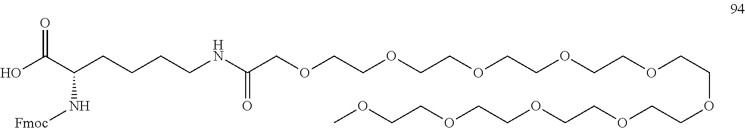

To a solution of sodium carbonate (11.98 g, 113.020 mmol) and sodium hydroxide (2.71 g, 67.812 mmol) in water (250 mL) were added compound 93 (28.32 g, 76.853 mmol) and compound 92 (22.83 g, 45.208 mmol). The mixture was stirred at room temperature for 3 h, and then concentrated under reduced pressure. The resulting residue was purified by prep-HPLC to give 94 (11.28 g, 29% yield) as a yellow oil. ESI MS m/z: calcd for C₄₂H₆₄N₂O₁₅ [M+H]⁺: 837.52 found 837.80.

### Example 68: Synthesis of ((S)-2-((S)-37-((((9H-fluoren-9-yl)methoxy)carbonyl)amino)-31,38-dioxo-2,5,8,11,14,17,20,23,26,29-decaoxa-32,39-diazatritetracontan-43-amido)-5-(tert-butoxy)-5-oxopentanoyl)-L-alanyl-L-alanine-CTC resin (96)

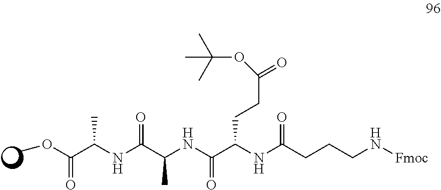

A peptide synthesizer glass vessel (Chemgalss, 100-mL) was charged with compound 48. Fmoc deprotection was performed using 10% piperidine/DMF solution (40 mL). The resin was then washed with DMF (3×50 mL). And then compound 95 (2.93 g, 9.000 mmol) dissolved in 50 mL of anhydrous DMF, HOBt (1.22 g, 9.000 mmol), and DIC (1.14 g, 9.000 mmol) were added. The reaction was run overnight and the resin was washed with DMF (3×50 mL) to give compound 96.

### Example 69: ((S)-2-((S)-37-((((9H-fluoren-9-yl)methoxy)carbonyl)amino)-31,38-dioxo-2,5,8,11,14,17,20,23,26,29-decaoxa-32,39-diazatritetracontan-43-amido)-5-(tert-butoxy)-5-oxopentanoyl)-L-alanyl-L-alanine-CTC resin (97)

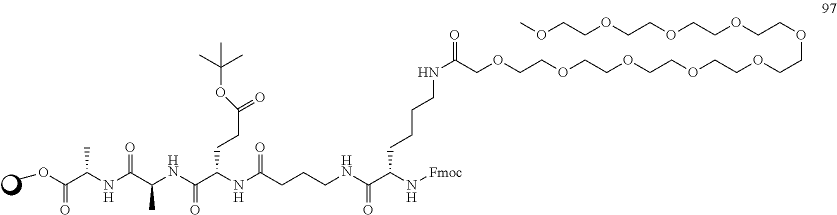

A peptide synthesizer glass vessel (Chemgalss, 100-mL) was charged with compound 96. Fmoc deprotection was performed using 10% piperidine/DMF solution (40 mL) The resin was then washed with DMF (3×50 mL). And then compound 94 (4.02 g, 4.800 mmol) dissolved in 50 mL of anhydrous DMF, HOBt (0.65 g, 4.800 mmol), and DIC (0.65 g, 4.800 mmol) were added. The reaction was run overnight and the resin was washed with DMF (3×50 mL) to give compound 97.

### Example 70: ((S)-2-((S)-37-((((9H-fluoren-9-yl)methoxy)carbonyl)amino)-31,38-dioxo-2,5,8,11,14,17,20,23,26,29-decaoxa-32,39-diazatritetracontan-43-amido)-5-(tert-butoxy)-5-oxopentanoyl)-L-alanyl-L-alanine (98)

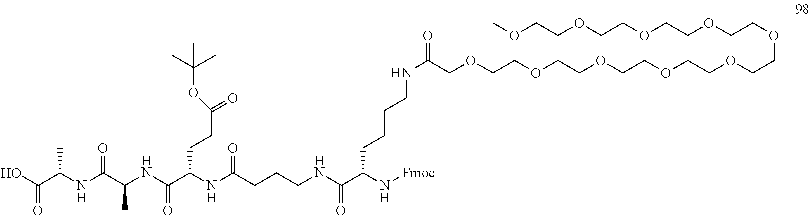

A peptide synthesizer glass vessel (Chemgalss, 100-mL) was charged with compound 97. The resin cleavage was performed using 1,1,1,3,3,3-hexafluoropropan-2-ol (15 mL) and DCM (35 mL), followed by a DCM wash (3×50 mL). The deprotection eluent was evaporated under reduced pressure to give 98 (7.25 g, 96% yield) as a white oil. ESI MS m/z: calcd for C₆₁H₉₆N₆O₂₁ [M+H]⁺: 1249.66; found 1249.81.

### Example 71: tert-butyl (37S,45S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (99)

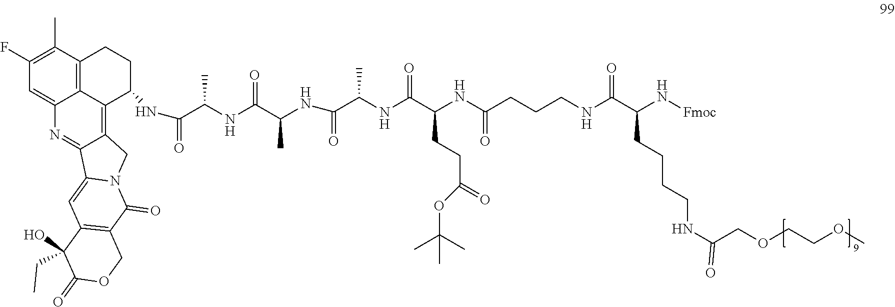

To a solution of compound 98 (1793 mg, 1.579 mmol), compound 56 (800 mg, 1.579 mmol) and HATU (631 mg, 1.658 mmol) in DMF (30 mL) was added DIEA (408 mg, 3.159 mmol). The reaction mixture was stirred at room temperature for 1.0 h, concentrated under reduced pressure to give 99 (2.74 g, 100% yield) as a yellow oil. ESI MS m/z: calcd for C₈₈H₁₂₁FN₁₀O₂₅ [M+H]⁺: 1737.85; found 1737.22.

### Example 72: (37S,45S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oic acid (100)

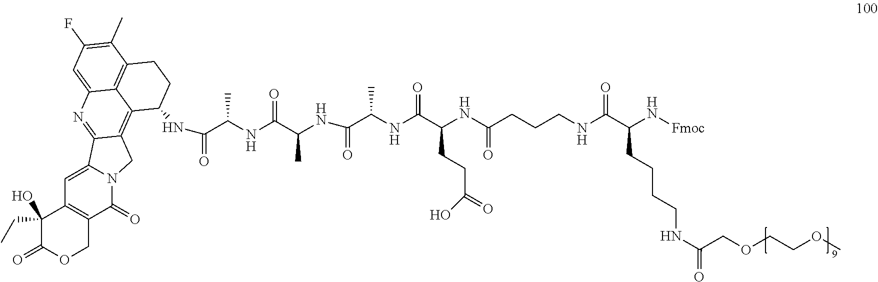

To a solution of compound 99 (2.74 g, 1.577 mmol) in DCM (15 mL) was added TFA (8 mL). The mixture was stirred at room temperature for 3 h, and then concentrated under reduced pressure. The resulting residue was purified by prep-HPLC to give 100 (1.50 g, 56% yield) as a yellow solid. ESI MS m/z: calcd for C₈₄H₁₁₃FN₁₀O₂₅ [M+H]⁺: 1681.79; found 1681.17.

### Example 73: perfluorophenyl (37S,45S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (101)

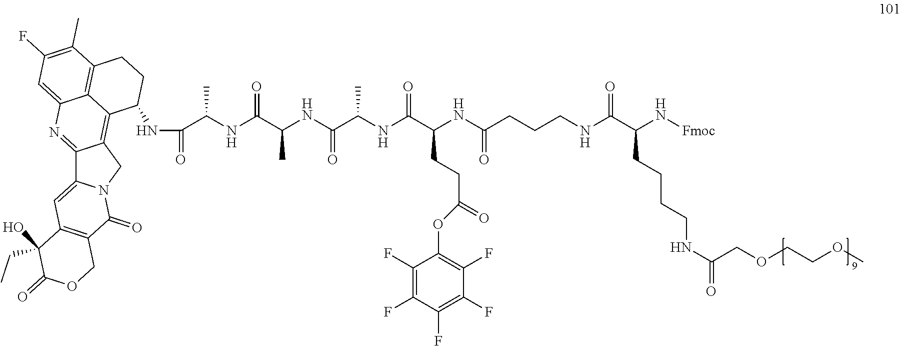

To a solution of compound 100 (1000 mg, 0.595 mmol) and PFP—OH (131 mg, 0.714 mmol) in DCM (10 mL) was added EDCI (171 mg, 0.892 mmol) at room temperature. The mixture was stirred at room temperature for 1.0 h, washed with brine (10 mL×2), then concentrated under reduced pressure. The resulting residue was purified by prep-HPLC to give 101 (880 mg, 80% yield) as a yellow solid. ESI MS m/z: calcd for C₉₀H₁₁₂F6N₁₀O₂₅ [M+H]⁺: 1847.77; found 1848.13.

### Example 74: N-(4-((37S,45S,50S,53S)-37-((((9H-fluoren-9-yl)methoxy)carbonyl)amino)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (102)

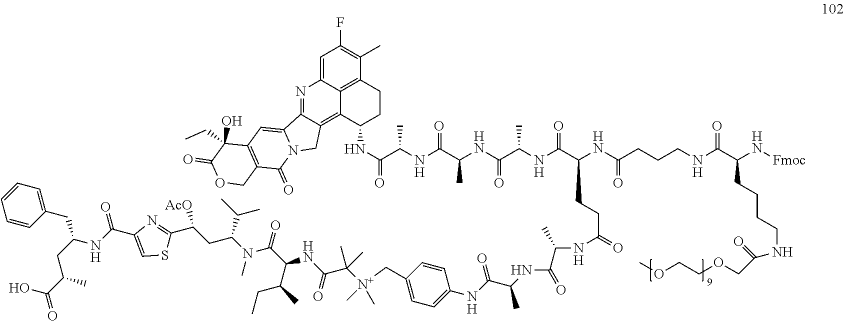

To a solution of compound 101 (400 mg, 0.216 mmol) and compound 39 (250 mg, 0.259 mmol) in DMF (6 mL) was added DIEA (56 mg, 0.433 mmol). The mixture was stirred at RT for 1 h, concentrated under reduced pressure to give 102 (570 mg, 100% yield) as a yellow oil. ESI: m/z: calcd for C₁₃₄H₁₈₆FN₁₈O₃₃S [M]²⁺: 1313.16; found 1313.21.

### Example 75: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((37S,45S,50S,53S)-37-amino-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50,53-dimethyl-31,38,43,48,51-pentaoxo-2,5,8,11,14,17,20,23,26,29-decaoxa-32,39,44,49,52-pentaazatetrapentacontan-54-amido)benzyl)-N,N,2-trimethyl-1-oxopropan-2-aminium (103)

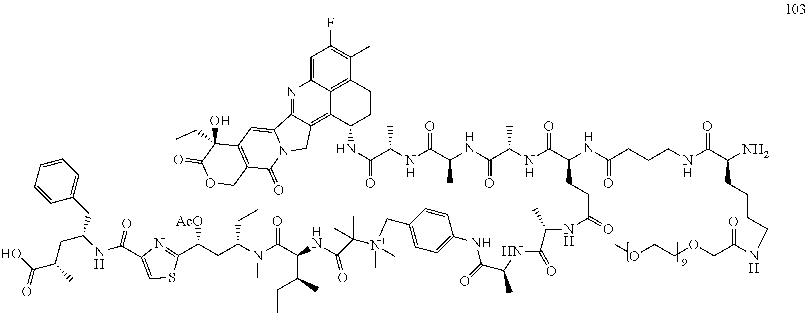

To a solution of compound 103 (570 mg, 0.217 mmol) in DMF (10 mL) was added piperidine (1 mL). The mixture was stirred at room temperature for 0.5 h, and then concentrated under reduced pressure. The resulting residue was purified by prep-HPLC to give 103 (380 mg, 72% yield) as a yellow solid. ESI MS m/z: calcd for C₁₁₉H₁₇₆FN₈O₃₁ S [M+H]²⁺: 1202.12; found 1202.22.

### Example 76: N,N′-(((((2S,5S,10S,18S,26R,27S,35S,43S,48S,51S)-26,27-bis(2,5-dioxo-2,5-dihydro-1H-pyrrol-1-yl)-10,43-bis(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-2,5,48,51-tetramethyl-4,7,12,17,20,25,28,33,36,41,46,49-dodecaoxo-18,35-bis(31-oxo-2,5,8,11,14,17,20,23,26,29-decaoxa-32-azahexatriacontan-36-yl)-3,6,11,16,19,24,29,34,37,42,47,50-dodecaazadopentacontanedioyl)bis(azanediyl)) bis(4,1-phenylene))bis(methylene))bis(1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium) (105)

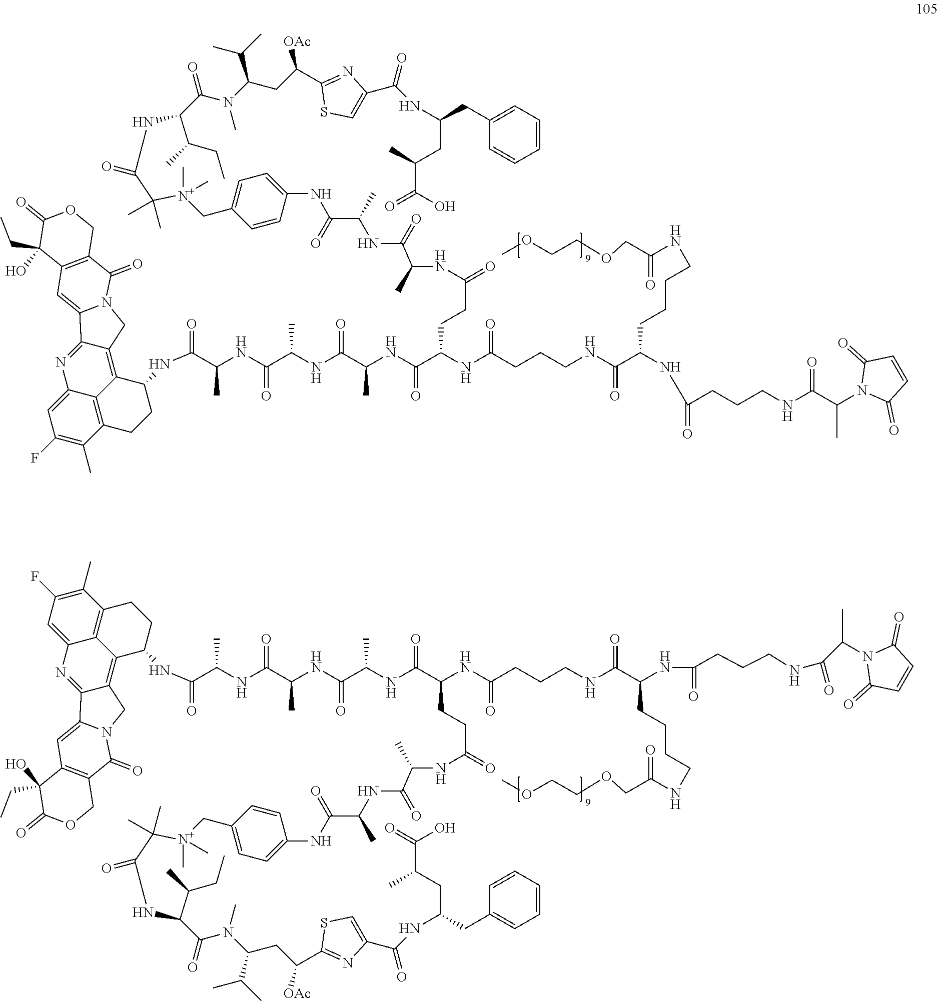

To a solution of compound 103 (80 mg, 0.033 mmol) and compound 104 (14 mg, 0.017 mmol) in DMF (2 mL) was added DIPEA (9 mg, 0.067 mmol). The mixture was stirred at RT for 2 h and purified by prep-HPLC to afford 105 (29 mg, 29% yield) as a white solid. ESI: m/z: calcd for C₂₅₈H₃₇₀F₂N₄₀O₇₀S₂⁺ [M]³⁺: 1750.20; found 1750.76.

### Example 77: 1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((37S,45S,50S,53S)-37-(4-(3,4-bis(2,5-dioxo-2,5-dihydro-1H-pyrrol-1-yl)-2,5-dioxopyrrolidin-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50,53-dimethyl-31,38,43,48,51-pentaoxo-2,5,8,11,14,17,20,23,26,29-decaoxa-32,39,44,49,52-pentaazatetrapentacontan-54-amido)benzyl)-N,N,2-trimethyl-1-oxopropan-2-aminium (107)

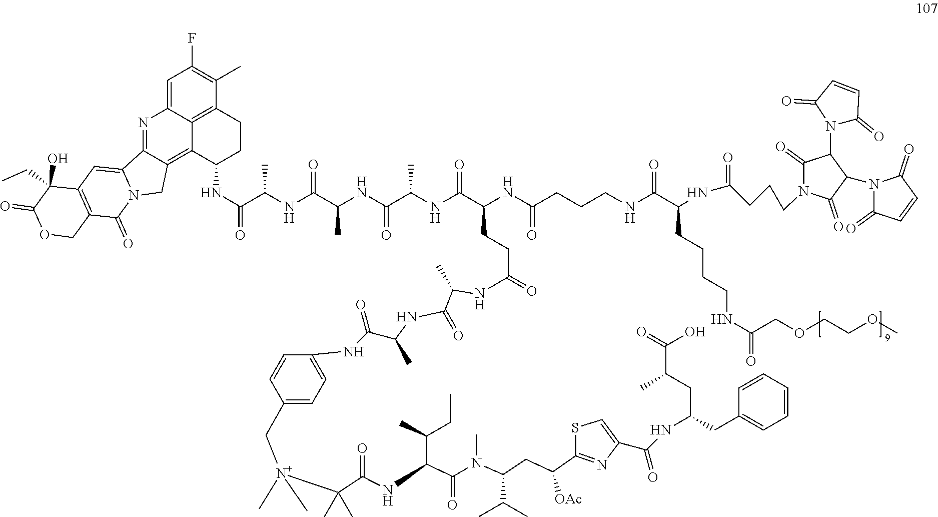

To a solution of compound 103 (80 mg, 0.033 mmol) and compound 85 (18 mg, 0.033 mmol) in DMF (2 mL) was added DIPEA (8 mg, 0.066 mmol). The mixture was stirred at RT for 1 h, and then purified by prep-HPLC to afford 107 (34 mg, 37% yield) as a white solid. ESI: m/z: calcd for C₁₃₅H₁₈₇FN₂₁O₃₈S [M]²⁺: 1380.65, found 1380.65.

### Example 78: (((9H-fluoren-9-yl)methoxy)carbonyl)-L-valine-CTC resin (110)

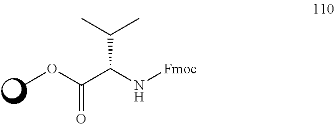

A peptide synthesizer glass vessel (Chemgalss, 100-mL) was charged with 2-chlorotrityl chloride resin (4.00 g, 1.5 g/mmol), compound 109 (4.07 g, 12.000 mmol) dissolved in 50 mL of anhydrous DCM and DIPEA (3.10 g, 24.000 mmol). The reaction was run overnight and the resin was washed with DCM (3×50 mL), followed by DMF (2×50 mL) to give compound 110.

### Example 79: ((S)-2-((((9H-fluoren-9-yl)methoxy)carbonyl)amino)-5-(tert-butoxy)-5-oxopentanoyl)-L-valine-CTC resin (112)

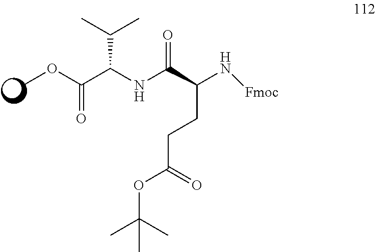

A peptide synthesizer glass vessel (Chemgalss, 100-mL) was charged with compound 110. Fmoc deprotection was performed using 10% piperidine/DMF solution (40 mL). The resin was then washed with DMF (3×50 mL) and then compound 111 (3.83 g, 9.000 mmol) dissolved in 50 mL of anhydrous DMF, HOBt (1.22 g, 9.000 mmol), and DIC (1.14 g, 9.000 mmol) were added. The reaction was run overnight and the resin was washed with DMF (3×50 mL) to give compound 112.

### Example 80: f ((S)-2-(4-((((9H-fluoren-9-yl)methoxy)carbonyl)amino) butanamido)-5-(tert-butoxy)-5-oxopentanoyl)-L-valine-CTC resin (113)

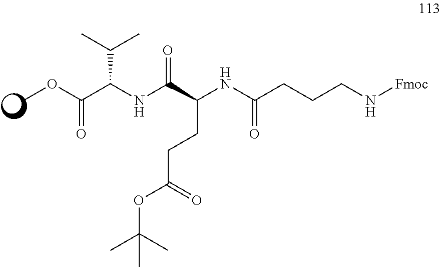

A peptide synthesizer glass vessel (Chemgalss, 100-mL) was charged with compound 112. Fmoc deprotection was performed using 10% piperidine/DMF solution (40 mL). The resin was then washed with DMF (3×50 mL). And then compound 95 (2.93 g, 9.000 mmol) dissolved in 50 mL of anhydrous DMF, HOBt (1.22 g, 9.000 mmol), and DIC (1.14 g, 9.000 mmol) were added. The reaction was run overnight and the resin was washed with DMF (3×50 mL) to give compound 113.

### Example 81: ((S)-2-((S)-37-((((9H-fluoren-9-yl)methoxy)carbonyl)amino)-31,38-dioxo-2,5,8,11,14,17,20,23,26,29-decaoxa-32,39-diazatritetracontan-43-amido)-5-(tert-butoxy)-5-oxopentanoyl)-L-valine-CTC resin (115)

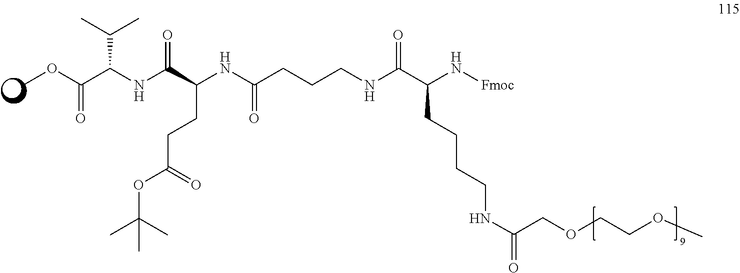

A peptide synthesizer glass vessel (Chemgalss, 100-mL) was charged with compound 113. Fmoc deprotection was performed using 10% piperidine/DMF solution (40 mL). The resin was then washed with DMF (3×50 mL). And then compound 94 (5.02 g, 6.000 mmol) was dissolved in 50 mL of anhydrous DMF, and HOBt (0.81 g, 6.000 mmol), DIC (0.76 g, 6.000 mmol) were added. The reaction was run overnight and the resin was washed with DMF (3×50 mL) to give compound 115.

### Example 82: ((S)-2-((S)-37-((((9H-fluoren-9-yl)methoxy)carbonyl)amino)-31,38-dioxo-2,5,8,11,14,17,20,23,26,29-decaoxa-32,39-diazatritetracontan-43-amido)-5-(tert-butoxy)-5-oxopentanoyl)-L-valine (116)

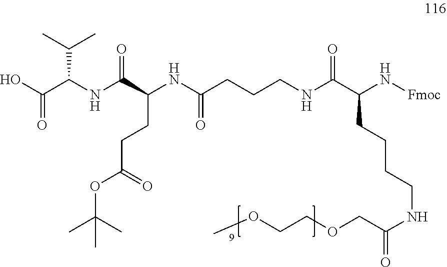

A peptide synthesizer glass vessel (Chemgalss, 100-mL) was charged with compound 115. The resin cleavage was performed using 1,1,1,3,3,3-hexafluoropropan-2-ol (15 mL) and DCM (35 mL), followed by a DCM wash (3×50 mL). The deprotection eluent was evaporated under reduced pressure to give 116 (6.15 g, 84% yield) as a yellow oil. ESI MS m/z: calcd for C₆₀H₉₅N₅O₂₀ [M+H]⁺: 1206.66; found 1205.82.

### Example 83: tert-butyl (37S,45S)-45-(((S)-1-(((S)-1-((3-((2,5,8,11,14,17,20,23-octaoxapentacosan-25-yl)carbamoyl)-4-(hydroxymethyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oate (118)

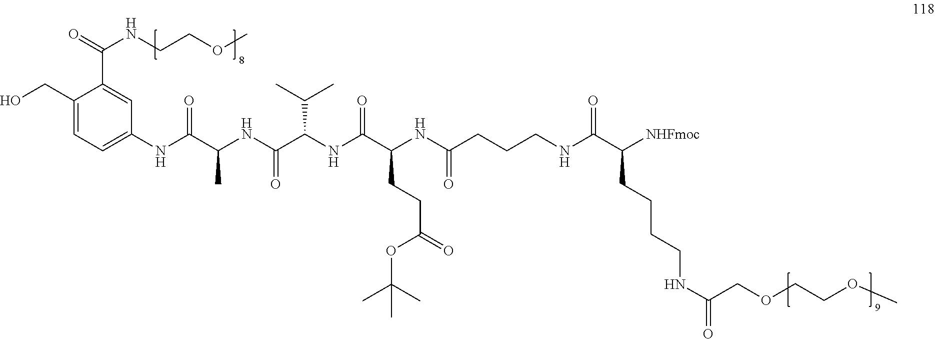

To a solution of compound 116 (2.66 g, 2.203 mmol), compound 117 (1.33 g, 2.203 mmol) and HATU (0.84 g, 2.203 mmol) in DMF (30 mL) was added DIEA (0.56 g, 4.406 mmol). The reaction mixture was stirred at room temperature for 1.0 h, concentrated under reduced pressure. The resulting residue was purified by prep-HPLC to give 118 (1.60 g, 40% yield) as a yellow oil. ESI MS m/z: calcd for C₈₈H₁₄₂N₈O₃₀ [M+H]⁺: 1791.98; found 1792.27.

### Example 84: tert-butyl (37S,45S)-45-(((S)-1-(((S)-1-((3-((2,5,8,11,14,17,20,23-octaoxapentacosan-25-yl)carbamoyl)-4-((((4-nitrophenoxy)carbonyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oate (119)

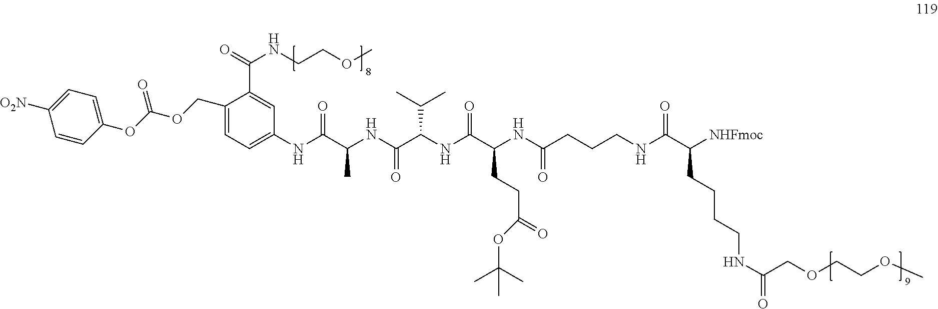

To a solution of compound 118 (1.60 g, 0.900 mmol), 4-nitrophenyl carbonochloridate (0.73 g, 3.600 mmol) in DCM (20 mL) was added pyridine (0.57 g, 7.200 mmol). The reaction mixture was stirred at room temperature for 1.0 h, concentrated under reduced pressure. The resulting residue was purified by flash column chromatography to give 119 (0.94 g, 53% yield) as a white solid. ESI MS m/z: calcd for C₉₅H₁₄₅N₉O₃₄ [M+H]⁺: 1956.99; found 1956.23.

### Example 85: (37S,45S)-45-(((S)-1-(((S)-1-((3-((2,5,8,11,14,17,20,23-octaoxapentacosan-25-yl)carbamoyl)-4-((((4-nitrophenoxy)carbonyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oic acid (120)

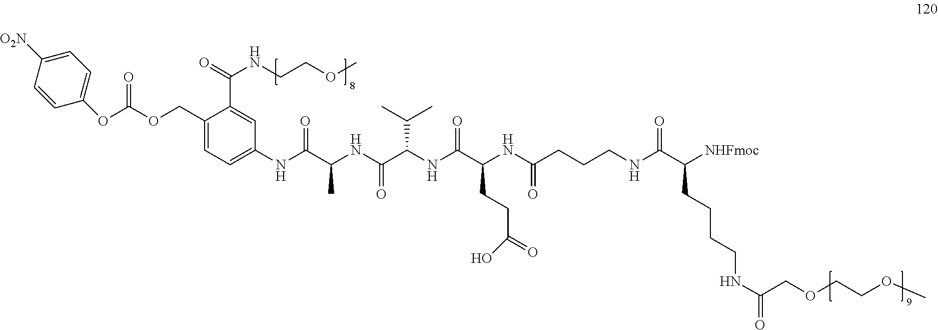

To a solution of compound 119 (0.94 g, 0.480 mmol) in DCM (4 mL) was added TFA (2 mL). The mixture was stirred at room temperature for 3 h, and then concentrated under reduced pressure. The resulting residue was purified by prep-HPLC to give 120 (738 mg, 81% yield) as a yellow solid. ESI MS m/z: calcd for C₉₁H₁₃₇N₉O₃₄ [M+H]⁺: 1900.93; found 1900.34.

### Example 86: (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oic acid (121)

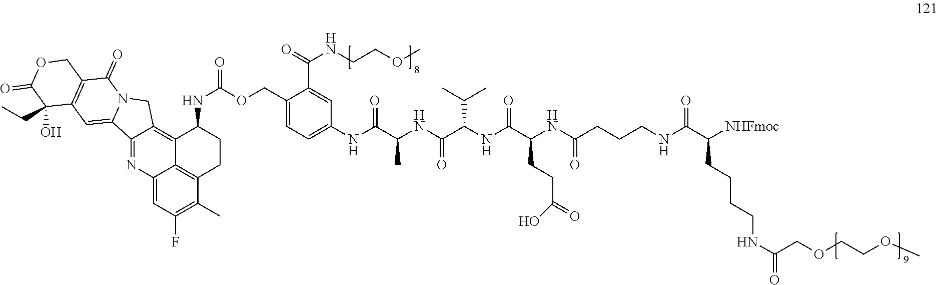

To a solution of compound 120 (369 mg, 0.194 mmol) and Exatecan (103 mg, 0.194 mmol) in DMF (5 mL) were added DIEA (25 mg, 0.388 mmol) and HOBt (26 mg, 0.194 mmol). The mixture was stirred at RT for 1 h, and concentrated under reduced pressure to give 121 (270 mg, 62% yield) as a yellow solid. ESI: m/z: calcd for C₁₀₉H₁₅₄FN₁₁O₃₅S [M+H]⁺: 2197.06; found 2198.11.

### Example 87: perfluorophenyl (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oate (122)

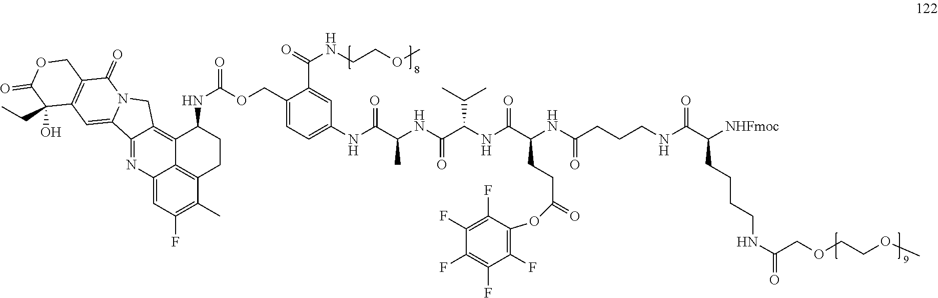

To a solution of compound 121 (270 mg, 0.123 mmol) and PFP—OH (270 mg, 0.147 mmol) in DCM (10 mL) was added EDCI (35 mg, 0.184 mmol) at room temperature. The mixture was stirred at room temperature for 1.0 h, washed with brine (10 mL×2), then concentrated under reduced pressure. The resulting residue was purified by prep-HPLC to give 122 (249 mg, 85% yield) as a yellow solid. ESI MS m/z: calcd for C₁₁₅H₁₅₃F₆N₁₁O₂₅ [M+H]⁺: 2363.04; found 2363.02.

### Example 88: N-(4-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (123)

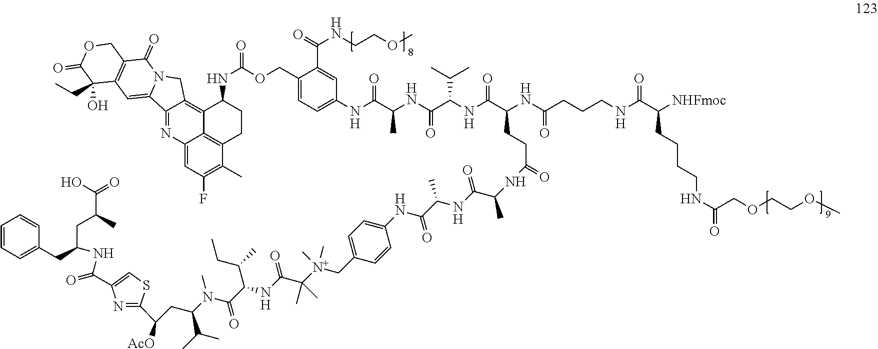

To a solution of compound 122 (249 mg, 0.105 mmol) and compound 39 (107 mg, 0.111 mmol) in DMF (5 mL) was added DIPEA (27 mg, 0.211 mmol). The mixture was stirred at RT for 1 h, concentrated under reduced pressure to give 123 (330 mg, 100% yield) as a yellow oil. ESI: m/z: calcd for C₁₅₈H₂₂₇FN₁₉O₄₃S [M]²⁺: 1570.80; found 1571.32.

### Example 89: N-(4-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-amino-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (124)

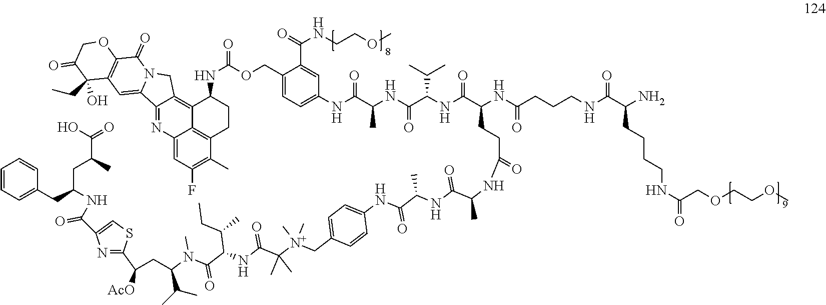

To a solution of compound 123 (330 mg, 0.106 mmol) in DMF (10 mL) was added piperidine (1 mL). The mixture was stirred at room temperature for 0.5 h, and then concentrated under reduced pressure. The resulting residue was purified by prep-HPLC to give 124 (210 mg, 67% yield) as a yellow solid. ESI MS m/z: calcd for C₁₄₄H₂₁₇FN₁₉O₄₁S [M+H]²⁺: 1459.76; found 1459.28.

### Example 90: N,N′-(((((2S,5S,10S,18S,26R,27S,35S,43S,48S,51S)-10,43-bis(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-26,27-bis(2,5-dioxo-2,5-dihydro-1H-pyrrol-1-yl)-2,5,48,51-tetramethyl-4,7,12,17,20,25,28,33,36,41,46,49-dodecaoxo-18,35-bis(31-oxo-2,5,8,11,14,17,20,23,26,29-decaoxa-32-azahexatriacontan-36-yl)-3,6,11,16,19,24,29,34,37,42,47,50-dodecaazadopentacontanedioyl)bis(azanediyl)) bis(4,1-phenylene))bis(methylene))bis(1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium) (125)

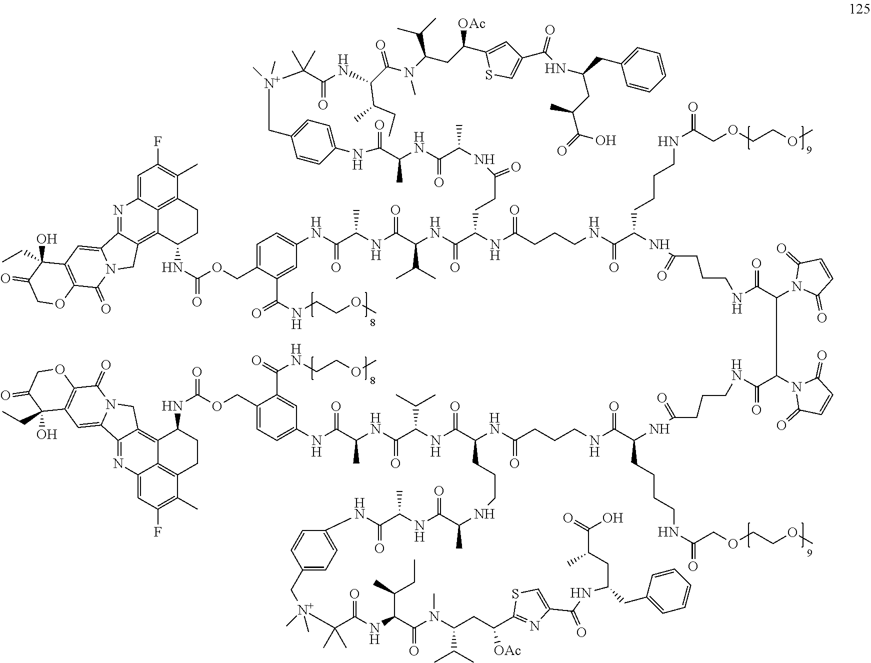

To a solution of compound 124 (100 mg, 0.034 mmol) and compound 111 (14 mg, 0.017 mmol) in DMF (2 mL) was added DIEA (9 mg, 0.068 mmol). The mixture was stirred at RT for 2 h, purified by prep-HPLC to afford 125 (91 mg, 76% yield) as a white solid. ESI: m/z: calcd for C₃₀₈H₄₅₂F₂N₄₂O₉₀S₂²⁺[M+H]³⁺: 2093.72; found 2094.73.

### Example 91: N-(4-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(3,4-bis(2,5-dioxo-2,5-dihydro-1H-pyrrol-1-yl)-2,5-dioxopyrrolidin-1-yl) butanamido)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (127)

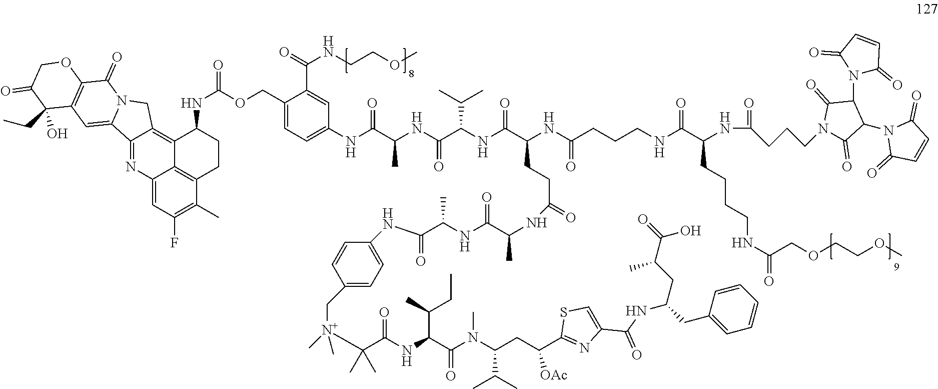

To a solution of compound 124 (80 mg, 0.027 mmol) and compound 85 (15 mg, 0.027 mmol) in DMF (2 mL) was added DIEA (7 mg, 0.055 mmol). The mixture was stirred at RT for 1 h, purified by prep-HPLC to afford 127 (60 mg, 66% yield) as a white solid. ESI: m/z: calcd for C₁₆₀H₂₂₈FN₂₂O₄₈S [M+H]²⁺: 1638.29, found 1639.28.

### Example 92: Synthesis of (37S,45S)-45-(((S)-1-(((S)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oic acid (129)

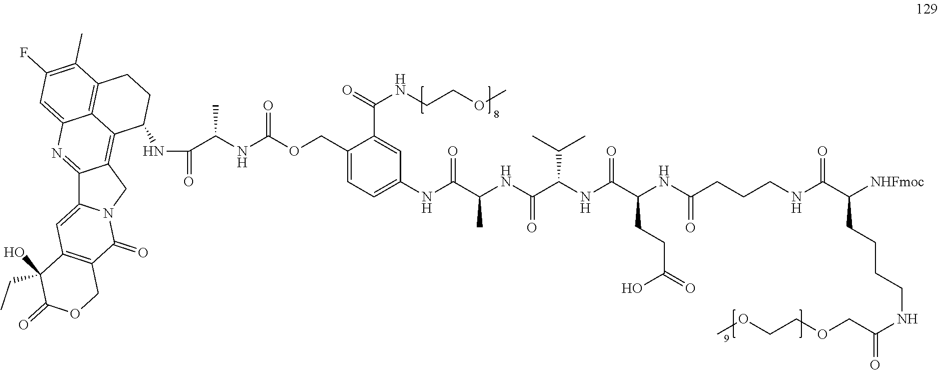

To a solution of compound 120 (369 mg, 0.194 mmol) and compound 56 (98 mg, 0.194 mmol) in DMF (5 mL) were added DIPEA (25 mg, 0.388 mmol) and HOBt (26 mg, 0.194 mmol). The mixture was stirred at RT for 1 h, concentrated under reduced pressure to give 129 (295 mg, 67% yield) as a yellow solid. ESI: m/z: calcd for C₁₁₂H₁₅₉FN₁₂O₃₆S [M]²⁺: 2268.10 found 2268.01.

### Example 93: perfluorophenyl (37S,45S)-45-(((S)-1-(((S)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-31,38,43-trioxo-2,5,8,11,14,17,20,23,26,29-decaoxa-32,39,44-triazaoctatetracontan-48-oate (130)

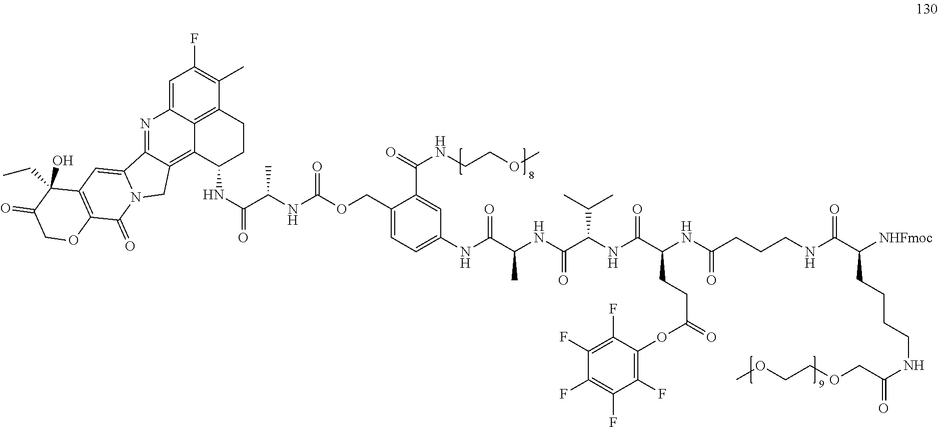

To a solution of compound 129 (295 mg, 0.130 mmol) and PFP—OH (28 mg, 0.156 mmol) in DCM (10 mL) was added EDCI (37 mg, 0.195 mmol) at room temperature. The mixture was stirred at room temperature for 1.0 h, washed with brine (10 mL×2), then concentrated under reduced pressure. The resulting residue was purified by prep-HPLC to give 130 (220 mg, 68% yield) as a yellow solid. ESI MS m/z: calcd for C₁₁₈H₁₅₈F₆N₁₂O₃₆ [M+H]⁺: 2434.08; found 2434.09.

### Example 94: N-(4-((37S,45S,50S,53S)-45-(((S)-1-(((S)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-37-((((9H-fluoren-9-yl)methoxy)carbonyl)amino)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (131)

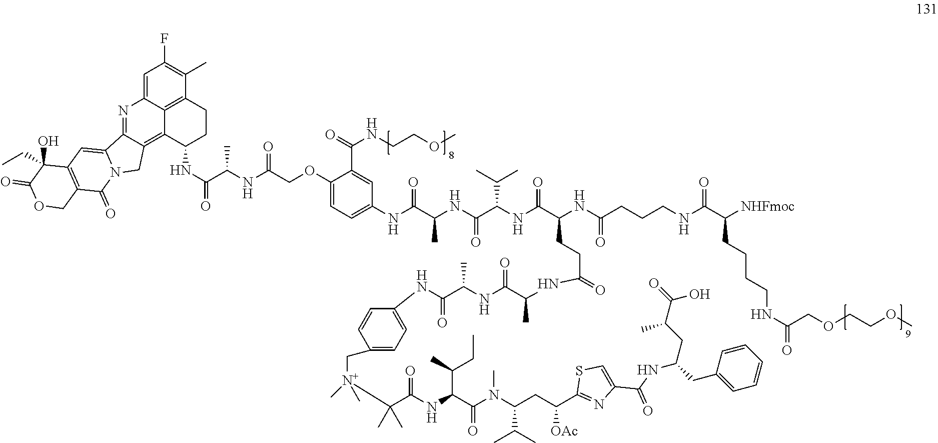

To a solution of compound 130 (212 mg, 0.087 mmol) and compound 130 (101 mg, 0.104 mmol) in DMF (5 mL) was added DIPEA (23 mg, 0.174 mmol). The mixture was stirred at RT for 1 h, concentrated under reduced pressure to give 131 (280 mg, 100% yield) as a yellow oil. ESI: m/z: calcd for C₁₆₂H₂₃₂FN₂₀O₄₄S [M]²⁺: 1606.31; found 1607.31.

### Example 95: N-(4-((37S,45S,50S,53S)-45-(((S)-1-(((S)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-37-amino-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (132)

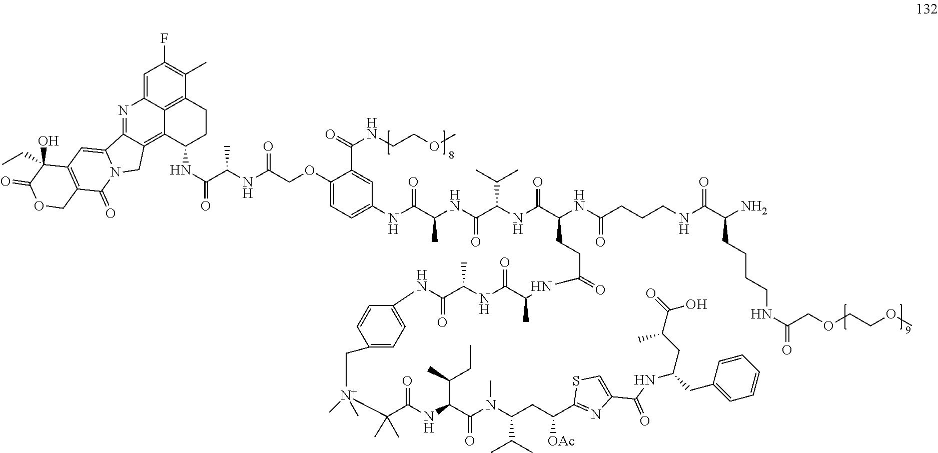

To a solution of compound 131 (280 mg, 0.087 mmol) in DMF (10 mL) was added piperidine (1 mL). The mixture was stirred at room temperature for 0.5 h, and then concentrated under reduced pressure. The resulting residue was purified by prep-HPLC to give 132 (130 mg, 49% yield) as a yellow solid. ESI MS m/z: calcd for C₁₄₇H₂₂₂FN₂₀O₄₂S [M]²⁺: 1495.28; found 1495.72.

### Example 96: N,N′-(((((2S,5S,10S,18S,26R,27S,35S,43S,48S,51S)-10,43-bis(((S)-1-(((S)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-26,27-bis(2,5-dioxo-2,5-dihydro-1H-pyrrol-1-yl)-2,5,48,51-tetramethyl-4,7,12,17,20,25,28,33,36,41,46,49-dodecaoxo-18,35-bis(31-oxo-2,5,8,11,14,17,20,23,26,29-decaoxa-32-azahexatriacontan-36-yl)-3,6,11,16,19,24,29,34,37,42,47,50-dodecaazadopentacontanedioyl)bis(azanediyl)) bis(4,1-phenylene))bis(methylene))bis(1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium) (133)

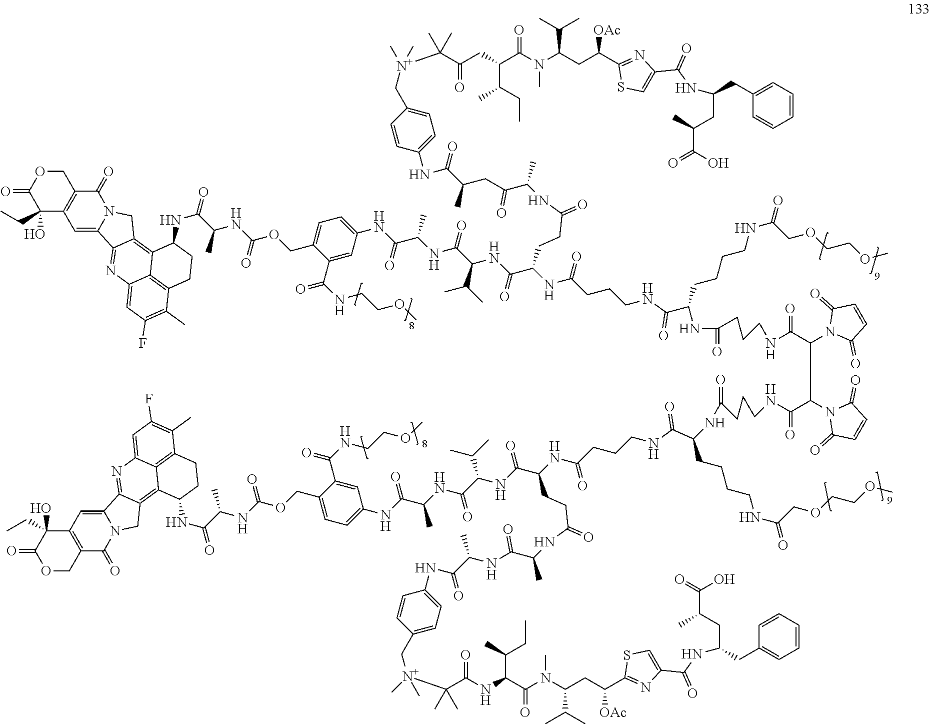

To a solution of compound 132 (70 mg, 0.023 mmol) and compound 111 (10 mg, 0.012 mmol) in DMF (2 mL) was added DIEA (6 mg, 0.047 mmol). The mixture was stirred at RT for 2 h. The reaction mixture was purified by prep-HPLC to afford 133 (37 mg, 44% yield) as a white solid. ESI: m/z: calcd for C₃₁₄H₄₆₂F₂N₄₄O₉₂S₂ [M+H]³⁺: 2141.07; found 2142.93.

### Example 97: perfluorophenyl(S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-oate (136)

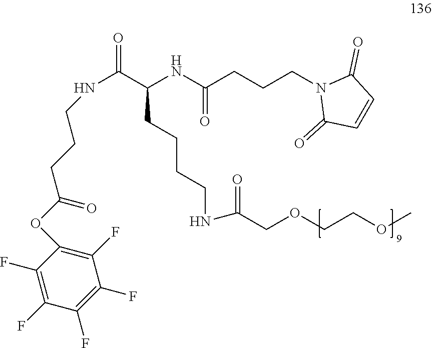

To solution of compound 135 (500 mg, 0.578 mmol) in DCM (5 mL) were added PFP—OH (138.32 mg, 0.075 mmol) and EDCI (166.22 mg, 0.087 mmol), and then stirred at room temperature for an hour. The organic phase was concentrated under reduced pressure. The resulting residue was purified by prep-HPLC to afford 136 (200 mg, 33% yield) as a white solid. ESI MS m/z: calcd for C₄₅H₆₇F₅N₄O₁₇ [M+H]⁺: 1031.44; found 1031.25.

### Example 98: perfluorophenyl(S)-5-(((S)-1-(((S)-1-((3-((2,5,8,11,14,17,20,23-octaoxapentacosan-25-yl)carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6,7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (138)

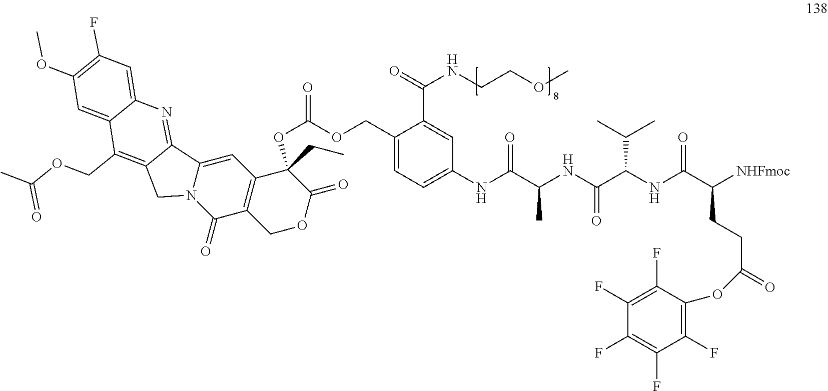

To solution of compound 137 (120 mg, 0.077 mmol) in DCM (1.2 mL) were added PFP—OH (15.7 mg, 0.085 mmol) and EDCI (17.83 mg, 0.093 mmol), and then stirred at room temperature for an hour. The organic phase was concentrated under reduced pressure. The resulting residue was purified by prep-HPLC to afford 138 (100 mg, 76% yield) as a yellow solid. ESI MS m/z: calcd for C₈₄H₉₃F₆N₇O₂₅ [M+H]⁺: 1715.61; found 1716.15.

### Example 99: N-(4-((2S,5S,10S,13S,16S)-17-((3-((2,5,8,11,14,17,20,23-octaoxapentacosan-25-yl)carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6,7]indolizino[1,2-b]quinolin-4-yl)oxy)carbonyl)oxy)methyl)phenyl)amino)-10-((((9H-fluoren-9-yl)methoxy)carbonyl)amino)-13-isopropyl-2,5,16-trimethyl-4,7,11,14,17-pentaoxo-3,6,12,15-tetraazaheptadecanamido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (139)

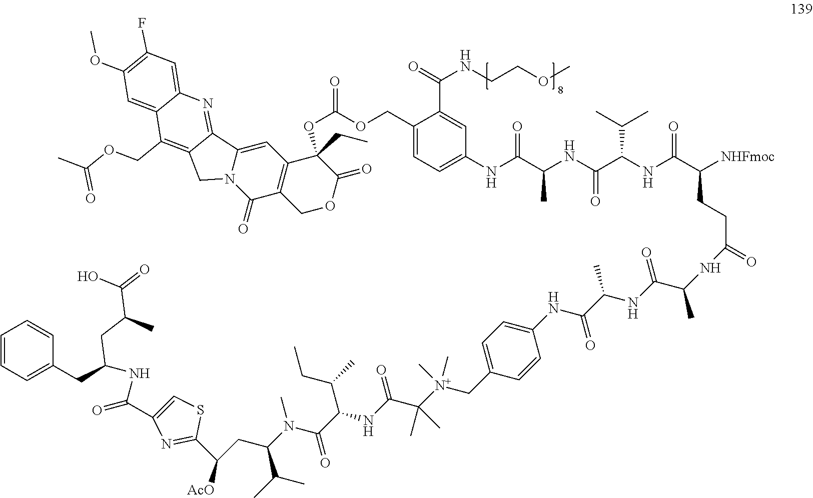

To solution of compound 138 (100 mg, 0.058 mmol) and compound 39 (59 mg, 0.061 mmol) in DMF (0.1 mL) was added DIEA (15.08 mg, 0.117 mmol). The reaction solution was stirred at room temperature for 1 h. The organic phase was concentrated under reduced pressure to give a crude product 139 (110 mg, 75% yield) as a yellow oil-like product. ESI MS m/z: calcd for C₁₂₈H₁₆₇FN₁₅O₃₃S [M]⁺2495.16; found 2494.52.

### Example 100: N-(4-((2S,5S,10S,13S,16S)-17-((3-((2,5,8,11,14,17,20,23-octaoxapentacosan-25-yl)carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6,7]indolizino[1,2-b]quinolin-4-yl)oxy)carbonyl)oxy)methyl)phenyl)amino)-10-amino-13-isopropyl-2,5,16-trimethyl-4,7,11,14,17-pentaoxo-3,6,12,15-tetraazaheptadecanamido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (140)

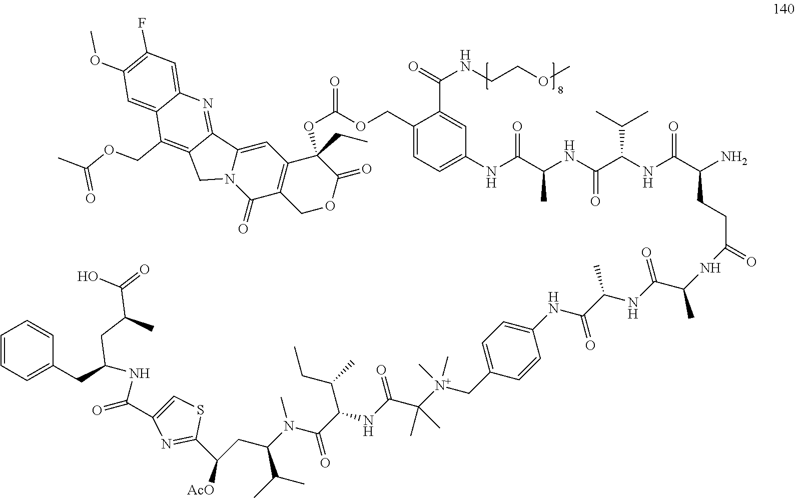

To solution of compound 139 (100 mg, 0.040 mmol) in DMF (0.1 mL) was added piperidine (18.6 mg, 0.220 mmol). The reaction solution was stirred at room temperature for half 1 h. The organic phase was concentrated under reduced pressure. The resulting residue was purified by prep-HPLC to afford 140 (52 mg, 57% yield) as a yellow solid. ESI MS m/z: calcd for C₁₁₃H₁₅₇FN₁₅O₃₁S [M]⁺: 2273.09; found 2272.73.

### Example 101: Synthesis of N-(4-((37S,45S,50S,53S)-45-(((S)-1-(((R)-1-((3-((2,5,8,11,14,17,20,23-octaoxapentacosan-25-yl)carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6,7]indolizino[1,2-b]quinolin-4-yl)oxy)carbonyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-50,53-dimethyl-31,38,43,48,51-pentaoxo-2,5,8,11,14,17,20,23,26,29-decaoxa-32,39,44,49,52-pentaazatetrapentacontan-54-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (141)

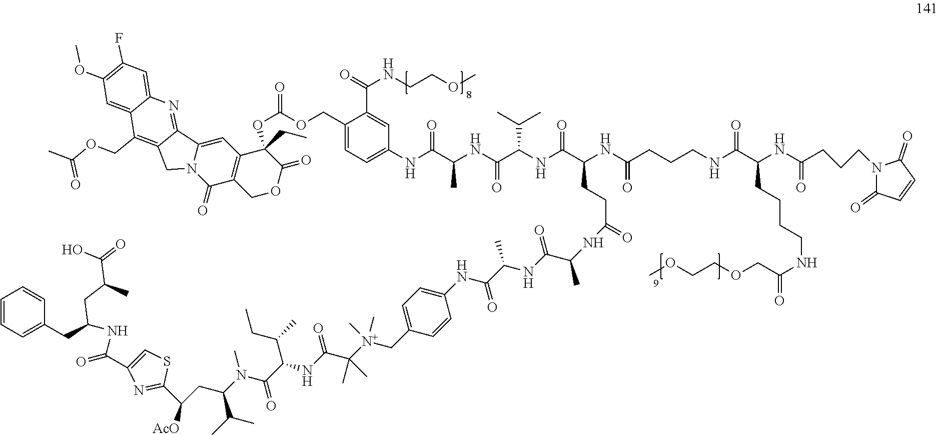

To solution of compound 140 (50 mg, 0.022 mmol) and compound 136 (24 mg, 0.023 mmol) in DMF (0.5 mL) was added DIEA (5.69 mg, 0.044 mmol). The reaction solution was stirred at room temperature for 1 h. The organic phase was concentrated under reduced pressure. The resulting residue was purified by prep-HPLC to afford 141 (17 mg, 24% yield) as light yellow solid. ESI MS m/z: calcd for C₁₅₂H₂₂₃FN₁₉O₄₇ [M+H]2+1560.21; found 1560.41.

### Example 102:1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N-(4-((37S,45S,50S,53S)-37-(6-(3,4-dibromo-2,5-dioxo-2,5-dihydro-1H-pyrrol-1-yl) hexanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50,53-dimethyl-31,38,43,48,51-pentaoxo-2,5,8,11,14,17,20,23,26,29-decaoxa-32,39,44,49,52-pentaazatetrapentacontan-54-amido)benzyl)-N,N,2-trimethyl-1-oxopropan-2-aminium (143)

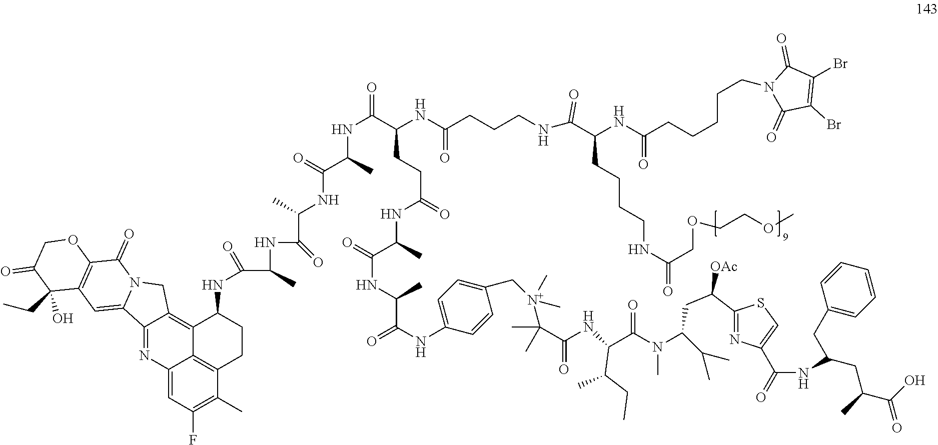

To a solution of compound 103 (90 mg, 0.037 mmol), 6-(3,4-Dibromo-2,5-dioxo-2,5-dihydro-1H-pyrrol-1-yl) hexanoic acid (15 mg, 0.041 mmol) and HATU (19 mg, 0.049 mmol) in DMF (2 mL) was added 2,6-dimethylpyridine (5 mg, 0.049 mmol). The reaction mixture was purified by prep-HPLC to afford 143 (20 mg, 19% yield) as a white solid. ESI: m/z: calcd for C₁₂₉H₁₈₅Br₂FN₁₉O₃₄S [M+H]²⁺: 1376.57; found 1377.88.

### Example 103: tert-butyl (37S,45S)-49-(3-(tert-butoxy)-3-oxopropyl)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43,48-tetraoxo-2,5,8,11,14,17,20,23,26,29-decaoxa-32,39,44,49-tetraazadopentacontan-52-oate (146)

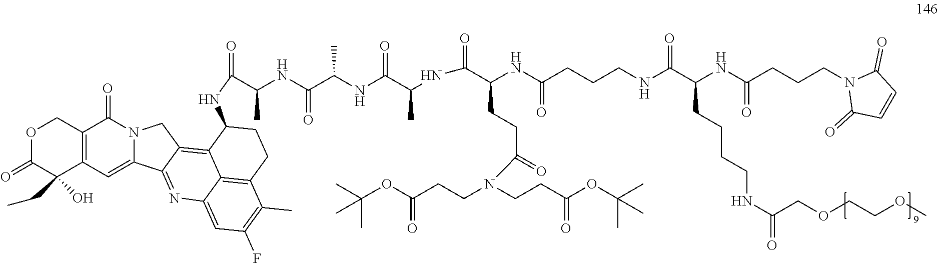

To a solution of compound 145 (0.30 g, 0.185 mmol) in DCM (10 mL) at room temperature were added compound 52 (51 mg, 0.185 mmol), HATU (85 mg, 0.222 mmol) and DIEA (36 mg, 0.277 mmol). The reaction mixture was stirred at room temperature for 0.5 h, and then washed with 1 N HCl (100 mL), dried over Na₂SO₄, filtered and concentrated under reduced pressure to afford 146 (0.35 g, 100% yield). ESI: m/z: calcd for C₉₁H₁₃₅FN₁₂O₂₉ [M+H]⁺: 1879.94; found 1879.94.

### Example 104: (37S,45S)-49-(2-carboxyethyl)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43,48-tetraoxo-2,5,8,11,14,17,20,23,26,29-decaoxa-32,39,44,49-tetraazadopentacontan-52-oic acid (147)

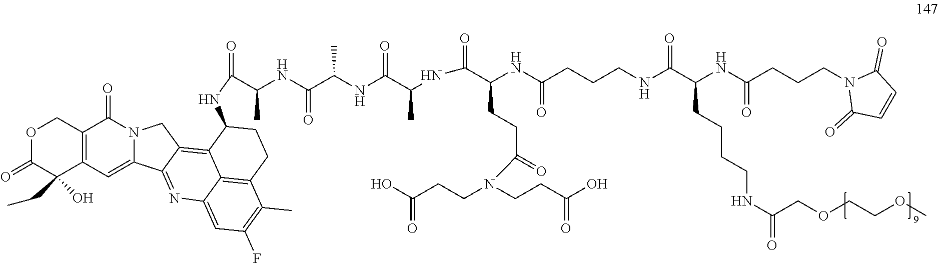

To a stirring solution of compound 146 (0.35 g, 0.186 mmol) in CH₂Cl₂ (5 mL) at room temperature was added TFA (5 mL). The reaction mixture was stirred at room temperature for 1 h, concentrated to afford 147 (0.32 g, 97% yield). ESI MS m/z: [M+H]⁺ calcd for C₈₃H₁₁₉FN₁₂O₂₉: 1767.82; found 1767.82.

### Example 105: perfluorophenyl (37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43,48-tetraoxo-49-(3-oxo-3-(perfluorophenoxy) propyl)-2,5,8,11,14,17,20,23,26,29-decaoxa-32,39,44,49-tetraazadopentacontan-52-oate (148)

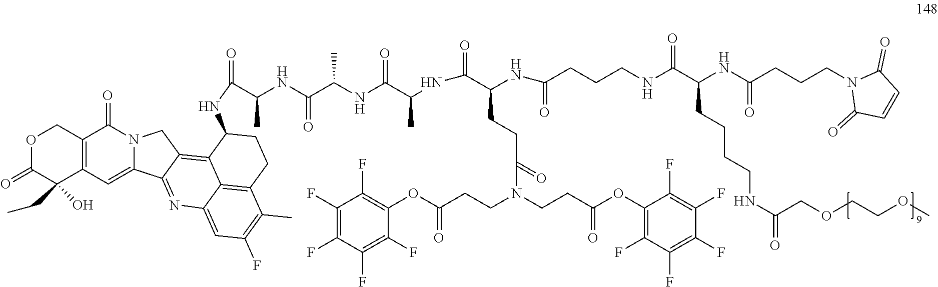

To a solution of compound 147 (0.32 g, 0.181 mmol) in DCM (15 mL) at room temperature were added EDCI (0.14 g, 0.724 mmol) and PFP—OH (0.10 g, 0.543 mmol). The reaction mixture was stirred at room temperature for 3 h, then concentrated under reduced pressure, purified by prep-HPLC, lyophilized to afford 148 (0.16 g, 42% yield). ESI MS m/z: calcd for C₉₅H₁₁₇F₁₁N₁₂O₂₉ [M+H]⁺: 2099.79; found 2099.79.

### Example 106: N,N′-(((((2S,5S,15S,18S)-10-((37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oyl)-2,5,15,18-tetramethyl-4,7,13,16-tetraoxo-3,6,10,14,17-pentaazanonadecanedioyl)bis(azanediyl))bis(4,1-phenylene))bis(methylene))bis(1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium) (149)

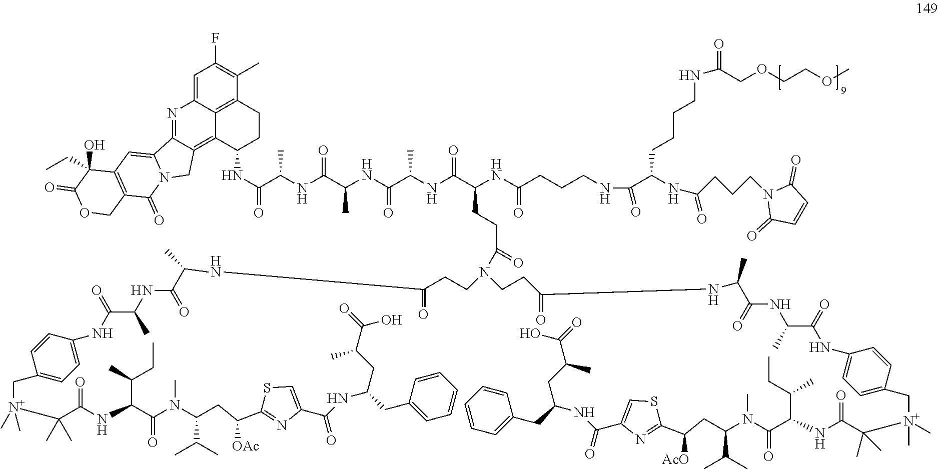

To a solution of compound 148 (105 mg, 0.050 mmol) in DMF (2 mL) at room temperature were added compound 39 (121 mg, 0.125 mmol) and DIEA (19 mg, 0.150 mmol). The reaction mixture was stirred at room temperature for 1.5 h, purified by prep-HPLC, lyophilized to afford 149 (60 mg, 27% yield). ESI MS m/z: calcd for C₁₈₃H₂₆₅FN28045S₂²⁺ [M]²⁺: 1828.94; found 1828.94.

### Example 107:2,5,8,11,14,17,20,23,26,29-decaoxahentriacontan-31-oyl chloride (159)

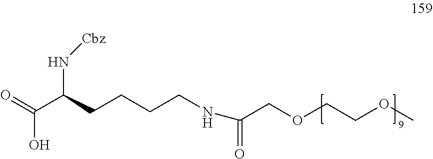

To a solution of sodium carbonate (16.12 g, 152.078 mmol) and sodium hydroxide (3.55 g, 88.712 mmol) in water (65 mL) were added compound 158 (26.64 g, 95.050 mmol) and compound 92 (32.00 g, 63.366 mmol). The mixture was stirred at room temperature for 3 h, and then concentrated under reduced pressure. The resulting residue was purified by prep-HPLC to give 159 (33.00 g, 69% yield) as a colorless oil. ESI MS m/z: calcd for C₃₅H₆₀N₂O₁₅ [M+H]⁺: 749.40; found 749.00.

### Example 108: tert-butyl((S)-37-(((benzyloxy)carbonyl)amino)-31-oxo-2,5,8,11,14,17,20,23,26,29-decaoxa-32-azaoctatriacontan-38-oyl)-L-valinate (161)

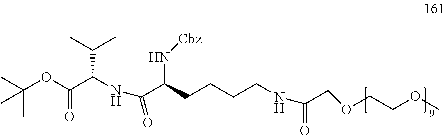

To a solution of compound 159 (6.00 g, 8.012 mmol), compound 160 (1.85 g, 8.813 mmol) and HATU (3.35 g, 8.813 mmol) in DMF (60 mL) was added DIEA (2.07 g, 16.024 mmol). The reaction mixture was stirred at room temperature for 1.0 h, concentrated under reduced pressure to give 161 (7.24 g, 100% yield) as a yellow oil. ESI MS m/z: calcd for C₄₄H₇₇N₃O₁₆ [M+H]⁺: 904.53; found 904.10.

### Example 109: ((S)-37-(((benzyloxy)carbonyl)amino)-31-oxo-2,5,8,11,14,17,20,23,26,29-decaoxa-32-azaoctatriacontan-38-oyl)-L-valine (162)

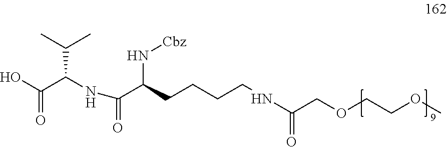

To a solution of compound 161 (7.24 g, 8.008 mmol) in DCM (50 mL) was added TFA (25 mL). The mixture was stirred at room temperature for 2 h, and then concentrated under reduced pressure. The resulting residue was purified by prep-HPLC to give 162 (6.50 g, 95% yield) as a colorless oil. ESI MS m/z: calcd for C₄₀H₆₉N₃O₁₆ [M+H]⁺: 848.47; found 848.17.

### Example 110: benzyl ((37S,40S,43S)-44-((3-((2,5,8,11,14,17,20,23-octaoxapentacosan-25-yl)carbamoyl)-4-(hydroxymethyl)phenyl)amino)-40-isopropyl-43-methyl-31,38,41,44-tetraoxo-2,5,8,11,14,17,20,23,26,29-decaoxa-32,39,42-triazatetratetracontan-37-yl)carbamate (164)

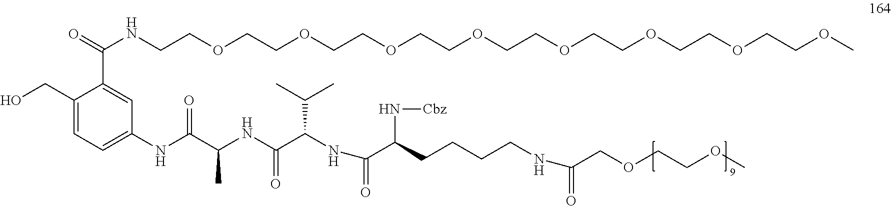

To a solution of compound 162 (2.81 g, 3.313 mmol), compound 117 (2.00 g, 3.313 mmol) and HATU (1.32 g, 3.478 mmol) in DMF (50 mL) was added DIEA (0.86 g, 6.626 mmol). The reaction mixture was stirred at room temperature for 1.0 h, concentrated under reduced pressure to give 164 (1.20 g, 25% yield) as a yellow oil. ESI MS m/z: calcd for C₆₈H₁₁₆N₆O₂₆ [M+H]⁺: 1433.79; found 1433.16.

### Example 111: benzyl ((37S,40S,43S)-44-((3-((2,5,8,11,14,17,20,23-octaoxapentacosan-25-yl)carbamoyl)-4-((((4-nitrophenoxy)carbonyl)oxy)methyl)phenyl)amino)-40-isopropyl-43-methyl-31,38,41,44-tetraoxo-2,5,8,11,14,17,20,23,26,29-decaoxa-32,39,42-triazatetratetracontan-37-yl)carbamate (166)

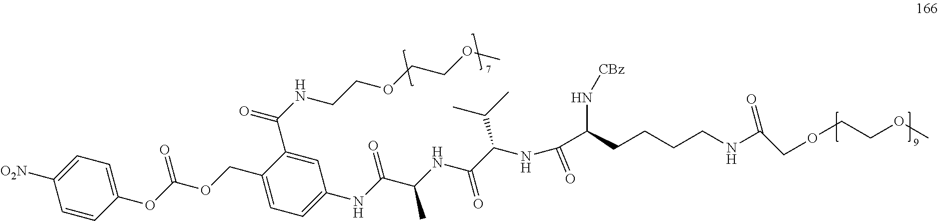

To a solution of compound 164 (1.10 g, 0.767 mmol) and 4-nitrophenyl carbonochloridate (0.31 g, 1.535 mmol) in DCM (20 mL) was added pyridine (0.24 g, 3.069 mmol). The reaction mixture was stirred at room temperature for 1.0 h, concentrated under reduced pressure. The resulting residue was purified by flash column chromatography to give 166 (817 mg, 66% yield) as a white solid. ESI MS m/z: calcd for C₇₅H₁₁₉N₇O₃₀ [M+H]⁺: 1598.80; found 1598.33.

### Example 112: benzyl ((37S,40S,43S)-44-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-40-isopropyl-43-methyl-31,38,41,44-tetraoxo-2,5,8,11,14,17,20,23,26,29-decaoxa-32,39,42-triazatetratetracontan-37-yl)carbamate (168)

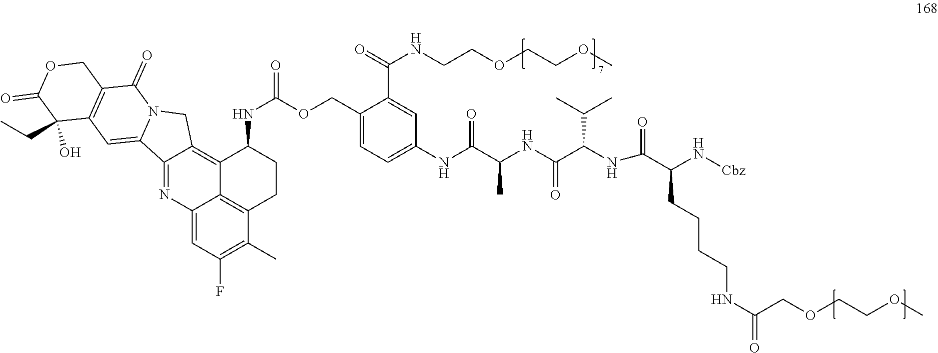

To a solution of compound 166 (817 mg, 0.511 mmol) and Exatecan (326 mg, 0.613 mmol) in DMF (10 mL) were added DIPEA (132 mg, 1.022 mmol) and HOBt (69 mg, 0.511 mmol). The mixture was stirred at RT for 1 h, concentrated under reduced pressure to give 168 (970 mg, 100% yield) as a yellow solid. ESI: m/z: calcd for C₉₃H₁₃₆FN₉O₃₁ [M+H]⁺: 1894.93; found 1895.05.

### Example 113: benzyl ((37S,40S,43S)-44-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-40-isopropyl-43-methyl-31,38,41,44-tetraoxo-2,5,8,11,14,17,20,23,26,29-decaoxa-32,39,42-triazatetratetracontan-37-yl)carbamate (169)

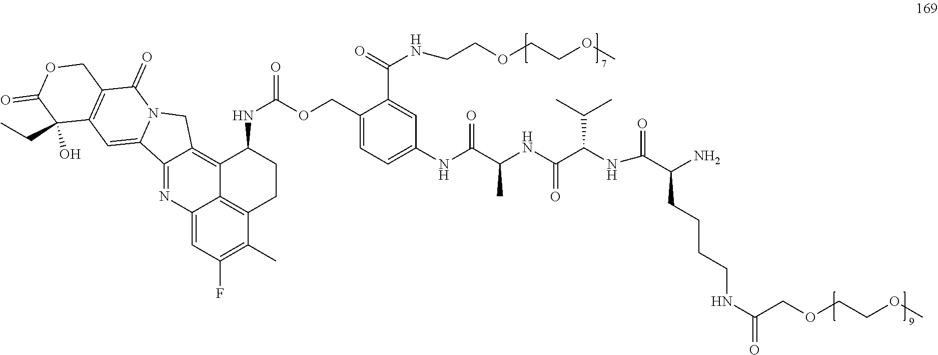

A solution of compound 168 (970 mg, 0.512 mmol) and Pd/C (55 mg) in methanol (10 mL) was stirred at room temperature for 2 h under H₂ protection. The mixture was filtered and the filtrate was concentrated and purified by prep-HPLC to give 169 (620 mg, 68% yield) as a yellow solid. ESI MS m/z: calcd for C₈₅H₁₃₀FN₉O₂₉ [M+H]⁺: 1760.90; found 1760.16.

### Example 114: (2S,4R)-5-(3-((37S,54S,57S,60S)-37-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-45,46-bis(2,5-dioxo-2,5-dihydro-1H-pyrrol-1-yl)-57-isopropyl-60-methyl-31,39,44,47,52,55,58-heptaoxo-54-(31-oxo-2,5,8,11,14,17,20,23,26,29-decaoxa-32-azahexatriacontan-36-yl)-2,5,8,11,14,17,20,23,26,29-decaoxa-32,38,43,48,53,56,59-heptaazahenhexacontan-61-amido)-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid (171)

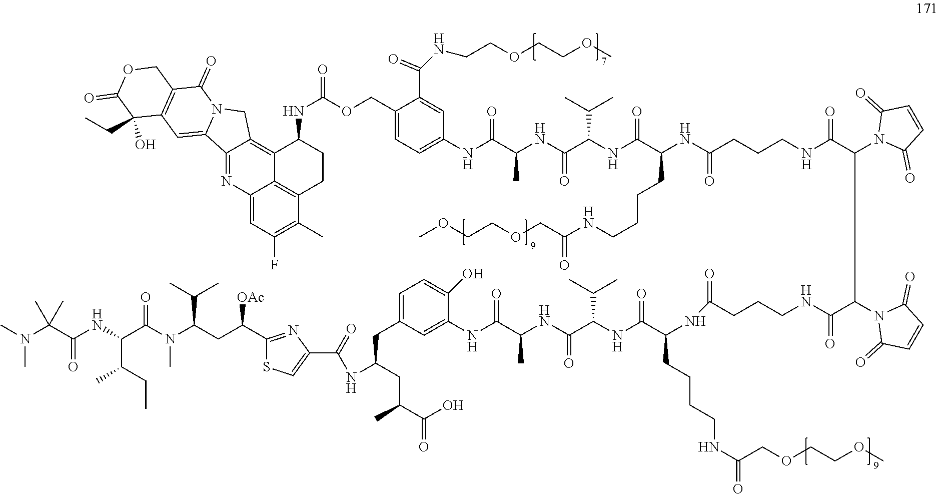

To a solution of compound 170 (374 mg, 0.247 mmol) and compound 111 (200 mg, 0.247 mmol) in DMF (2 mL) was added DIPEA (64 mg, 0.494 mmol). The mixture was stirred at RT for 1 h, and then compound 169 (348 mg, 0.198 mmol) and DIPEA (64 mg, 0.494 mmol) were added. The mixture was stirred at RT for 1 h, and purified by prep-HPLC to afford 171 (183 mg, 19% yield) as a white solid. ESI: m/z: calcd for C₁₇₇H₂₇₂FN₂₃O₅₉S [M+2H]²⁺: 1858.44; found 1858.65.

### Example 115: tert-butyl 1-(4-((2,2-dimethyl-4-oxo-3,8,11-trioxa-5-azatridecan-13-yl)carbamoyl)phenyl)-1-oxo-5,8,11,14-tetraoxa-2-azaheptadecan-17-oate (175)

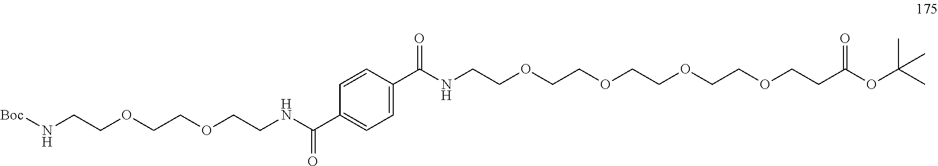 [SMILES: CC(C)(C)OC(=O)CCOCCOCCOCCOCCNC(=O)c1ccc(C(=O)NCCOCCOCCNC(=O)OC(C)(C)C)cc1]

To a solution of compound 173 (1.85 g, 4.667 mmol), compound 174 (1.500 g, 4.667 mmol) and HATU (1.86 g, 4.667 mmol) in DCM (30 mL) was added DIEA (0.90 g, 7.000 mmol) at room temperature. The mixture was stirred for 30 min before being concentrated under reduced pressure. The resulting residue was purified by flash column chromatography to afford 175 (3.27 g, 100% yield) as a yellow oil. ESI MS m/z: calcd for C₃₄H₅₈N₃O₁₂ [M+H]⁺: 700.39; found 700.35.

### Example 116:1-(4-((2-(2-(2-aminoethoxy) ethoxy)ethyl)carbamoyl)phenyl)-1-oxo-5,8,11,14-tetraoxa-2-azaheptadecan-17-oic acid (176)

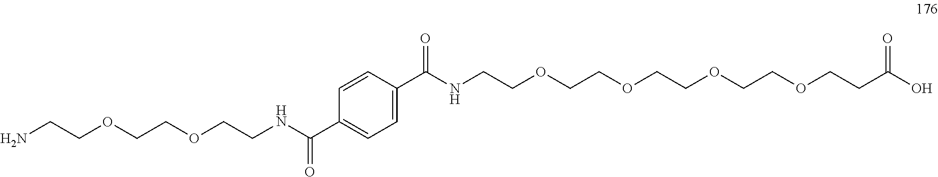 [SMILES: NCCOCCOCCNC(=O)c1ccc(C(=O)NCCOCCOCCOCCOCCC(=O)O)cc1]

To a solution of compound 175 (3.27 g, 4.672 mmol) in DCM (40 mL) was added TFA (30 mL). The reaction mixture was stirred at room temperature for 1.0 h, concentrated under reduced pressure. The resulting residue was purified by prep-HPLC to afford 6 (2.10 g, 82% yield) as a colorless oil. ESI MS m/z: calcd for C₂₅H₄₂N₃O₁₀ [M+H]⁺: 544.28; found 543.89

### Example 117: (S)-5-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)amino)-4-(4-(2,5-dioxo-2,5-dihydro-1H-pyrrol-1-yl) butanamido)-5-oxopentanoic acid (178)

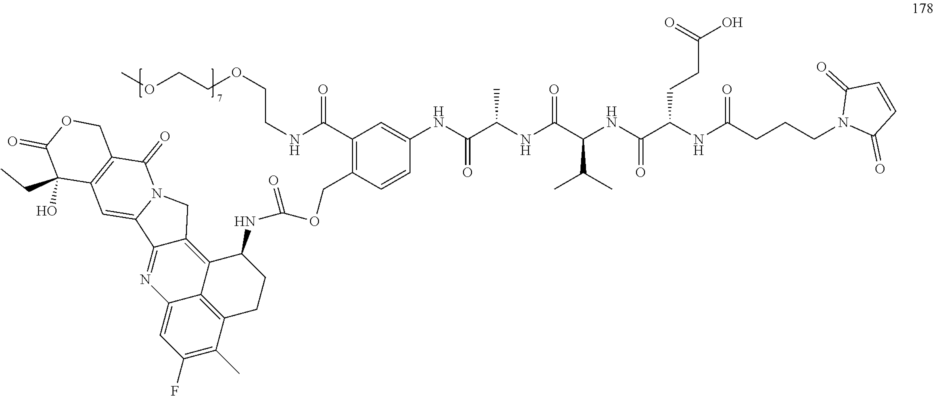

To a solution of compound 177 (2.34 g, 2.015 mmol), Exatecan (1.07 g, 2.015 mmol) and HOBt (0.27 g, 2.015 mmol) in DMF (25 mL) was added DIEA (1.04 g, 8.061 mmol). The reaction mixture was stirred at room temperature for 2.5 h, and then concentrated under reduced pressure. The resulting residue was purified by prep-HPLC to afford 178 (1.06 g, 36% yield) as a yellow solid. ESI MS m/z: calcd for C₇₁H₉₃FN₉O₂₃ [M+H]⁺: 1458.63; found 1457.96

### Example 118: perfluorophenyl(S)-5-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)amino)-4-(4-(2,5-dioxo-2,5-dihydro-1H-pyrrol-1-yl) butanamido)-5-oxopentanoate (179)

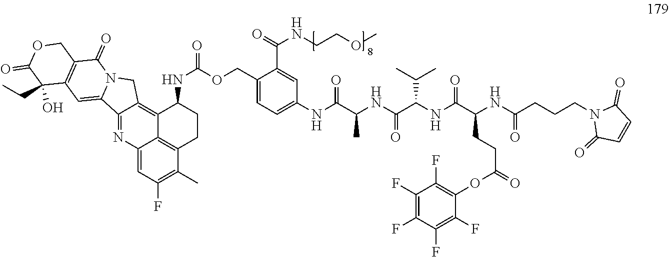

To a solution of compound 178 (1.06 g, 0.727 mmol), PFP—OH (0.17 g, 0.945 mmol) in DCM (20 mL) was added EDCI (0.21 g, 1.090 mmol). The reaction mixture was stirred at room temperature for 1.0 h, washed with brine, dried over Na₂SO₄, concentrated under reduced pressure to afford 179 (1.18 g, 0.727 mmol, 100% yield) as a yellow gum. ESI MS m/z: calcd for C₇₇H₉₂₃F₆N₉O₂₃ [M+H]⁺: 1624.61; found 1624.90

### Example 119:1-(4-(((13S,16S,19S)-20-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-13-(4-(2,5-dioxo-2,5-dihydro-1H-pyrrol-1-yl) butanamido)-16-isopropyl-19-methyl-10,14,17,20-tetraoxo-3,6-dioxa-9,15,18-triazaicosyl)carbamoyl)phenyl)-1-oxo-5,8,11,14-tetraoxa-2-azaheptadecan-17-oic acid (180)

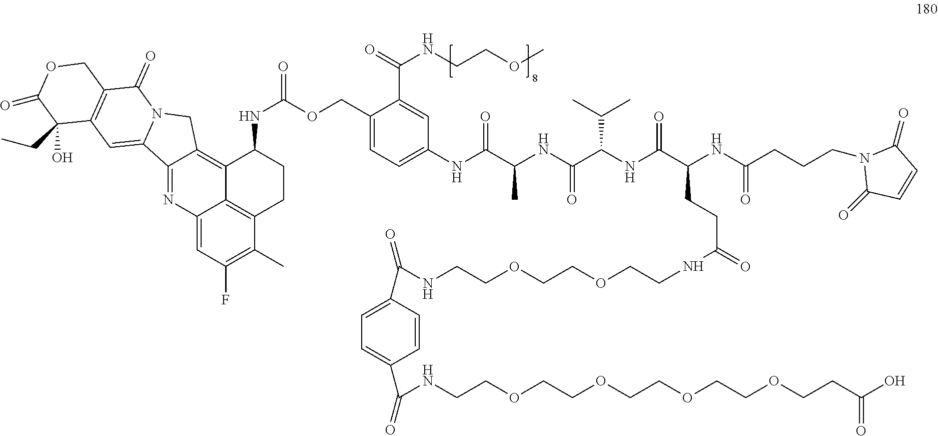

To a solution of compound 179 (1.18 g, 0.726 mmol), compound 176 (0.39 g, 0.726 mmol) in DMF (15 mL) was added DIEA (0.23 g, 1.816 mmol). The reaction mixture was stirred at room temperature for 1.0 h and then quenched by formic acid (0.5 mL), purified by prep-HPLC to afford 180 (790 mg, 19% yield) as a yellow solid. ESI MS m/z: calcd for C₉₆H₁₃₂FN₁₂O₃₂ [M+H]⁺: 1983.90; found 1983.95.

### Example 120: perfluorophenyl 1-(4-(((13S,16S,19S)-20-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-13-(4-(2,5-dioxo-2,5-dihydro-1H-pyrrol-1-yl) butanamido)-16-isopropyl-19-methyl-10,14,17,20-tetraoxo-3,6-dioxa-9,15,18-triazaicosyl)carbamoyl)phenyl)-1-oxo-5,8,11,14-tetraoxa-2-azaheptadecan-17-oate (181)

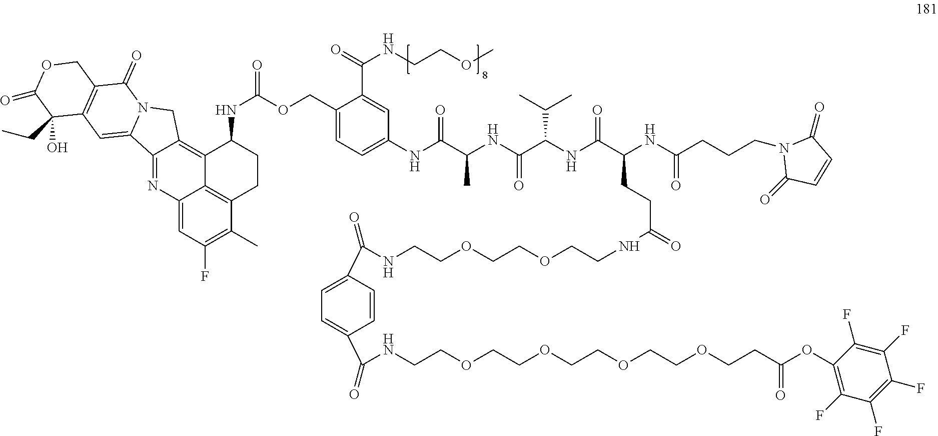

To a solution of compound 179 (200 mg, 0.101 mmol), PFP—OH (24 mg, 0.131 mmol) in DCM (2 mL) was added EDCI (29 mg, 0.151 mmol). The reaction mixture was stirred at room temperature for 1.0 h, washed with brine, dried over Na₂SO₄, concentrated under reduced pressure to afford 181 (216 mg, 99% yield) as a yellow gum. ESI MS m/z: calcd for C₁₀₂H₁₃₀F₆N₁₂O₃₂ [M+H]⁺: 2149.88; found 2150.35.

### Example 121: N-(4-((19S,22S)-1-(4-(((13S,16S,19S)-20-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-13-(4-(2,5-dioxo-2,5-dihydro-1H-pyrrol-1-yl) butanamido)-16-isopropyl-19-methyl-10,14,17,20-tetraoxo-3,6-dioxa-9,15,18-triazaicosyl)carbamoyl)phenyl)-19-isopropyl-22-methyl-1,17,20-trioxo-5,8,11,14-tetraoxa-2,18,21-triazatricosan-23-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (182)

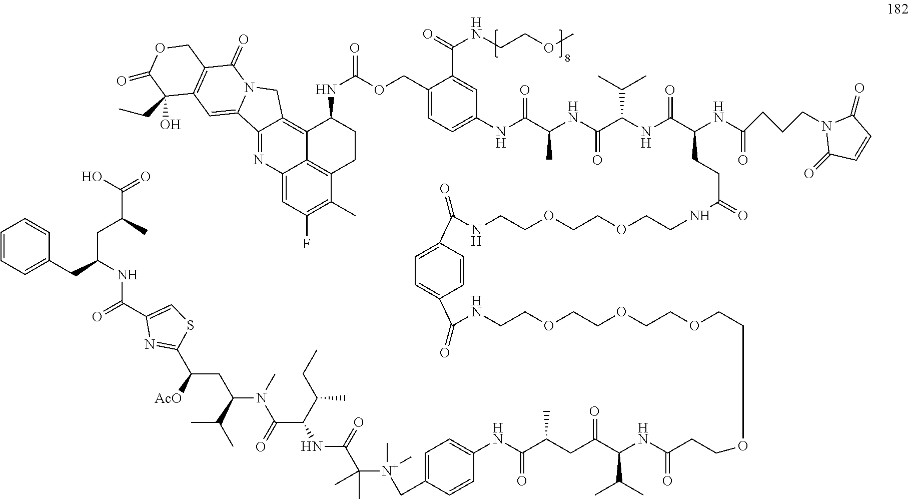

To a solution of compound 181 (125 mg, 0.058 mmol) and compound 39 (58 mg, 0.058 mmol) in DMF (2 mL) was added DIEA (7.51 mg, 0.058 mmol). The reaction solution was stirred at room temperature for 1 h and then purified by prep-HPLC to afford 182 (67 mg, 39% yield) as a white solid. ESI MS m/z: calcd for C₁₄₈H₂₀₈FN₂₀O₄₀S [M+H]²⁺: 1478.23 found 1478.7.

### Example 122: perfluorophenyl (37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43,48-tetraoxo-2,5,8,11,14,17,20,23,26,29,52,55,58,61,64,67,70,73-octadecaoxa-32,39,44,49-tetraazahexaheptacontan-76-oate (185)

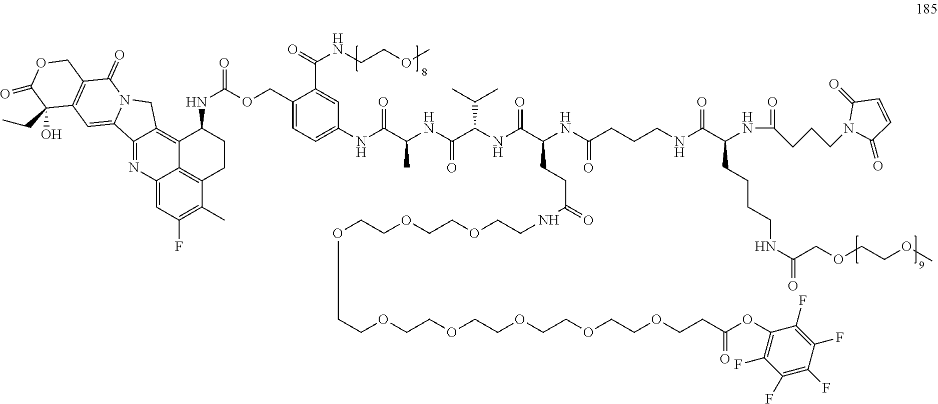

To a solution of compound 184 (200 mg, 0.078 mmol), PEP-OH (19 mg, 0.101 mmol) in DCM (2 mL) was added EDCI (22 mg, 0.117 mmol). The reaction mixture was stirred at room temperature for 1.0 h, washed with brine, dried over Na₂SO₄, concentrated under reduced pressure to afford 185 (200 mg, 0.073 mmol, 95% yield) as a yellow gum. ESI MS m/z: calcd for C₁₂₇H₁₈₇F₆N13045 [M+2H]²⁺: 1365.13; found 1364.83.

### Example 123: N-(4-((37S,45S,78S,81S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-78-isopropyl-81-methyl-31,38,43,48,76,79-hexaoxo-2,5,8,11,14,17,20,23,26,29,52,55,58,61,64,67,70,73-octadecaoxa-32,39,44,49,77,80-hexaazadooctacontan-82-amido)benzyl)-1-(((2S,3S)-1-(((1R,3R)-1-acetoxy-1-(4-(((2R,4S)-4-carboxy-1-phenylpentan-2-yl)carbamoyl)thiazol-2-yl)-4-methylpentan-3-yl)(methyl)amino)-3-methyl-1-oxopentan-2-yl)amino)-N,N,2-trimethyl-1-oxopropan-2-aminium (186)

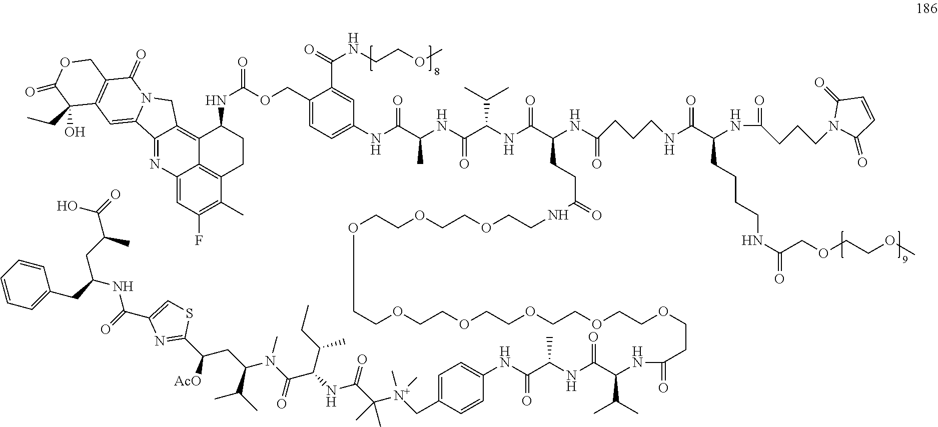

To a solution of compound 185 (115 mg, 0.042 mmol) and compound 39 (42 mg, 0.042 mmol) in DMF (2 mL) was added DIEA (5 mg, 0.042 mmol). The reaction solution was stirred at room temperature for 1 h. The residue was purified by prep-HPLC to afford 186 (80 mg, 53% yield) as a white solid. ESI MS m/z: calcd for C₁₇₃H₂₆₅FN₂₁O₅₃S [M+H]²⁺: 1767.92; found 1767.79.

### Example 130: N2-(((9H-fluoren-9-yl)methoxy)carbonyl)-N4-trityl-L-asparagine-CTC resin (202)

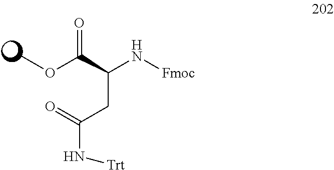

A peptide synthesizer glass vessel (Chemgalss, 100-mL) was charged with 2-chlorotrityl chloride resin (3.00 g, 1.5 mmol/g), compound 201 (5.37 g, 9.000 mmol) in 50 mL of anhydrous DCM and DIEA (2.33 g, 18.000 mmol). The reaction was run overnight and the resin was washed with DCM (3×50 mL), followed by DMF (2×50 mL) to give compound 202.

### Example 131: N4-trityl-L-asparagine-CTC resin (203)

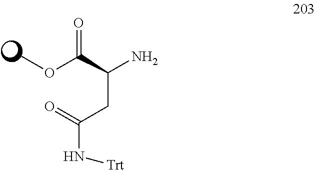

A peptide synthesizer glass vessel (Chemgalss, 100-mL) was charged with compound 202. Fmoc deprotection was performed using 10% piperidine/DMF solution (50 mL). The resin was then washed with DMF (3×50 mL) to give compound 203.

### Example 132: N4-trityl-L-asparagine-CTC resin (205)

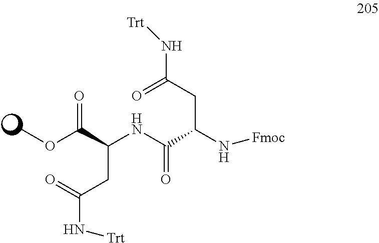

A peptide synthesizer glass vessel (Chemgalss, 100-mL) were charged with compound 203, compound 204 (4.03 g, 6.750 mmol) in 50 mL of anhydrous DMF and HOBt (0.91 g, 6.750 mmol), DIC (0.85 g, 6.750 mmol). The reaction was run overnight and the resin was washed with DMF (3×50 mL) to give compound 205.

### Example 133: N4-trityl-N2-(N4-trityl-L-asparaginyl)-L-asparagine-CTC resin (206)

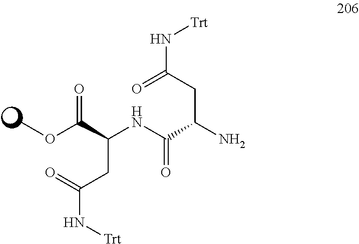

A peptide synthesizer glass vessel (Chemgalss, 100-mL) was charged with compound 205. Fmoc deprotection was performed using 10% piperidine/DMF solution (50 mL). The resin was then washed with DMF (3×50 mL) to give compound 206.

### Example 134: N2-(N2-((((9H-fluoren-9-yl)methoxy)carbonyl)-L-alanyl)-N4-trityl-L-asparaginyl)-N4-trityl-L-asparagine-CTC resin (208)

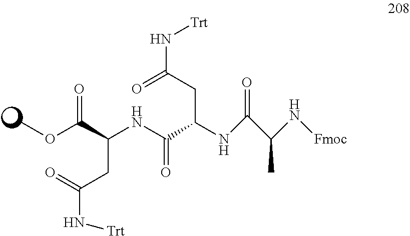

A peptide synthesizer glass vessel (Chemgalss, 100-mL) were charged with compound 206, compound 207 (2.10 g, 6.750 mmol) in 50 mL of anhydrous DMF, HOBt (0.91 g, 6.750 mmol) and DIC (0.85 g, 6.750 mmol). The reaction was run overnight and the resin was washed with DMF (3×50 mL) to give compound 208.

### Example 135: N2-(N2-(L-alanyl)-N4-trityl-L-asparaginyl)-N4-trityl-L-asparagine-CTC resin (209)

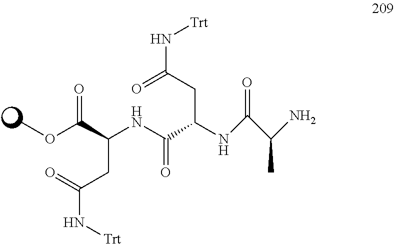

A peptide synthesizer glass vessel (Chemgalss, 100-mL) was charged with compound 208. Fmoc deprotection was performed using 10% piperidine/DMF solution (50 mL). The resin was then washed with DMF (3×50 mL) to give compound 209.

### Example 136: N2-(N2-(((9H-fluoren-9-yl)methoxy)carbonyl)-L-isoleucyl-L-alanyl-N4-trityl-L-asparaginyl)-N4-trityl-L-asparagine-CTC resin (211)

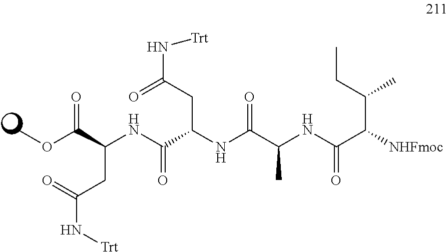

A peptide synthesizer glass vessel (Chemgalss, 100-mL) was charged with compound 209.

Compound 210 (2.39 g, 6.750 mmol) dissolved in 50 mL of anhydrous DMF, and HOBt (0.91 g, 6.750 mmol), DIC (0.85 g, 6.750 mmol) were added. The reaction was run overnight and the resin was washed with DMF (3×50 mL) to give compound 211.

### Example 137: N2-(N2-L-isoleucyl-L-alanyl-N4-trityl-L-asparaginyl)-N4-trityl-L-asparagine-CTC resin (212)

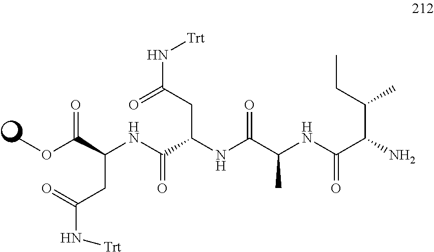

A peptide synthesizer glass vessel (Chemgalss, 100-mL) was charged with compound 211. Fmoc deprotection was performed using 10% piperidine/DMF solution (50 mL). The resin was then washed with DMF (3×50 mL) to give compound 212.

### Example 138: N2-(N2-(((9H-fluoren-9-yl)methoxy)carbonyl)-L-isoleucyl-L-isoleucyl-L-alanyl-N4-trityl-L-asparaginyl)-N4-trityl-L-asparagine-CTC resin (214)

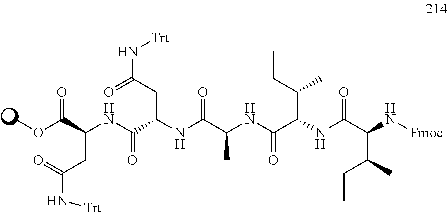

A peptide synthesizer glass vessel (Chemgalss, 100-mL) were charged with compound 212, compound 213 (2.39 g, 6.750 mmol) dissolved in 50 mL of anhydrous DMF, HOBt (0.91 g, 6.750 mmol), and DIC (0.85 g, 6.750 mmol). The reaction was run overnight and the resin was washed with DMF (3×50 mL) to give compound 214.

### Example 139: N2-(N2-L-isoleucyl-L-isoleucyl-L-alanyl-N4-trityl-L-asparaginyl)-N4-trityl-L-asparagine-CTC resin (215)

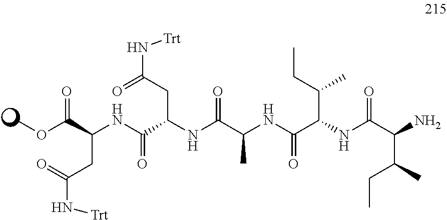

A peptide synthesizer glass vessel (Chemgalss, 100-mL) was charged with compound 214. Fmoc deprotection was performed using 10% piperidine/DMF solution (50 mL). The resin was then washed with DMF (3×50 mL) to give compound 215.

### Example 140: N2-(N2-N2-(((9H-fluoren-9-yl)methoxy)carbonyl)-N5-trityl-L-glutaminyl-L-isoleucyl-L-isoleucyl-L-alanyl-N4-trityl-L-asparaginyl)-N4-trityl-L-asparagine-CTC resin (217)

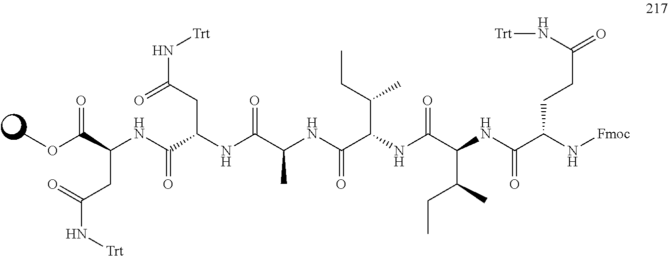

A peptide synthesizer glass vessel (Chemgalss, 100-mL) were charged with compound 215, compound 216 (4.12 g, 6.750 mmol) in 50 mL of anhydrous DMF, HOBt (0.91 g, 6.750 mmol), and DIC (0.85 g, 6.750 mmol). The reaction was run overnight and the resin was washed with DMF (3×50 mL) to give compound 217.

### Example 141: N4-trityl-N2-(N4-trityl-N2-N5-trityl-L-glutaminyl-L-isoleucyl-L-isoleucyl-L-alanyl-L-asparaginyl)-L-asparagine-CTC resin (218)

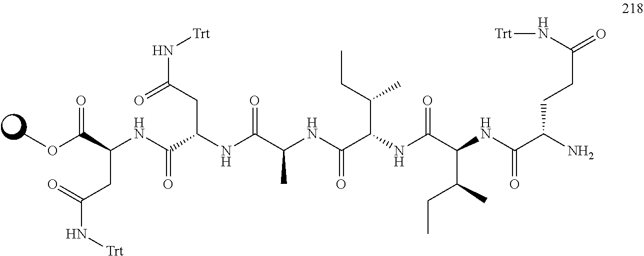

A peptide synthesizer glass vessel (Chemgalss, 100-mL) was charged with compound 217. Fmoc deprotection was performed using 10% piperidine/DMF solution (50 mL) The resin was then washed with DMF (3×50 mL) to give compound 218.

### Example 142: N2-(N2-N2-((S)-2-((((9H-fluoren-9-yl)methoxy)carbonyl)amino)-4-(tert-butoxy)-4-oxobutanoyl)-N5-trityl-L-glutaminyl-L-isoleucyl-L-isoleucyl-L-alanyl-N4-trityl-L-asparaginyl)-N4-trityl-L-asparagine-CTC resin (220)

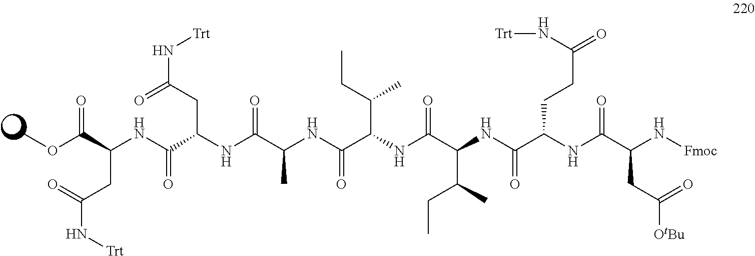

A peptide synthesizer glass vessel (Chemgalss, 100-mL) was charged with compound 218. Compound 219 (2.78 g, 6.750 mmol) dissolved in 50 mL of anhydrous DMF and HOBt (0.91 g, 6.750 mmol), DIC (0.85 g, 6.750 mmol) were added. The reaction was run overnight and the resin was washed with DMF (3×50 mL) to give compound 220.

### Example 143: N2-(N2-N2-((S)-2-amino-4-(tert-butoxy)-4-oxobutanoyl)-N5-trityl-L-glutaminyl-L-isoleucyl-L-isoleucyl-L-alanyl-N4-trityl-L-asparaginyl)-N4-trityl-L-asparagine-CTC resin (221)

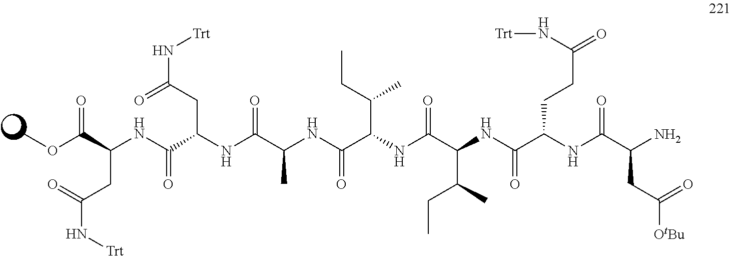

A peptide synthesizer glass vessel (Chemgalss, 100-mL) was charged with compound 220. Fmoc deprotection was performed using 10% piperidine/DMF solution (50 mL). The resin was then washed with DMF (3×50 mL) to give compound 221.

### Example 144: (2S,5S,8S,11S,14S,17S,20S)-20-amino-2,5-bis(2-amino-2-oxoethyl)-17-(3-amino-3-oxopropyl)-11,14-di((S)-sec-butyl)-8-methyl-4,7,10,13,16,19-hexaoxo-3,6,9,12, 15,18-hexaazadocosanedioic acid (222)

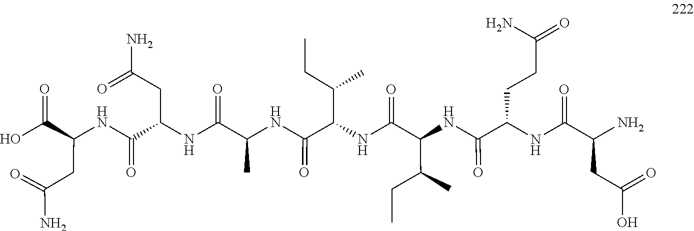 [SMILES: CC[C@H](C)[C@H](NC(=O)[C@H](CCC(N)=O)NC(=O)[C@@H](N)CC(=O)O)C(=O)N[C@H](C(=O)N[C@@H](C)C(=O)N[C@@H](CC(N)=O)C(=O)N[C@@H](CC(N)=O)C(=O)O)[C@@H](C)CC]

A peptide synthesizer glass vessel (Chemgalss, 100-mL) was charged with compound 221. TFA (20 mL) and DCM (40 mL) and triisopropylsilane (1 mL) were added. The reaction was run for 4 h and the resin was washed with DCM (3×50 mL). The mixture was concentrated under reduced pressure. The resulting residue was purified by prep-HPLC to give compound 222 (500 mg) as a white solid. ESI MS m/z: calcd for C₃₂H₅₄N₁₀O₁₃ [M+H]⁺: 787.39; found 786.89.

### Example 145: tert-butyl 1-(5-((37S,40S,43S)-37-(4-(2,5-dioxo-2,5-dihydro-1H-pyrrol-1-yl) butanamido)-40-isopropyl-43-methyl-31,38,41-trioxo-2,5,8,11,14,17,20,23,26,29-decaoxa-32,39,42-triazatetratetracontan-44-amido)-2-((((4-nitrophenoxy)carbonyl)oxy)methyl)phenyl)-1-oxo-5,8,11,14,17,20,23,26-octaoxa-2-azanonacosan-29-oate (224)

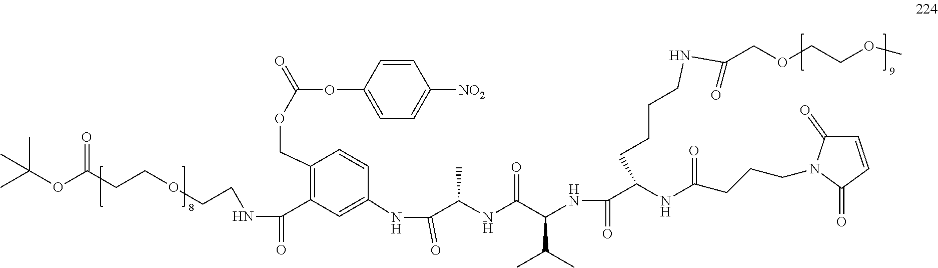

To a solution of compound 223 (800 mg, 0.507 mmol) and compound 202 (204 mg, 1.013 mmol) in DCM (10 mL) was added Pyridine (62 mg, 1.013 mmol) and DIEA (4.45 g, 34.426 mmol). The reaction mixture was stirred at room temperature for 1 h, and then concentrated under reduced pressure. The resulting residue was purified by flash column chromatography to give 224 (703 mg, 79% yield) as a colorless oil. ESI MS m/z: calcd for C₈₁H₁₃₀N₈O₃₃ [M+H]⁺: 1743.87; found 1743.40.

### Example 146: 1-(5-((37S,40S,43S)-37-(4-(2,5-dioxo-2,5-dihydro-1H-pyrrol-1-yl) butanamido)-40-isopropyl-43-methyl-31,38,41-trioxo-2,5,8,11,14,17,20,23,26,29-decaoxa-32,39,42-triazatetratetracontan-44-amido)-2-((((4-nitrophenoxy)carbonyl)oxy)methyl)phenyl)-1-oxo-5,8,11,14,17,20,23,26-octaoxa-2-azanonacosan-29-oic acid (225)

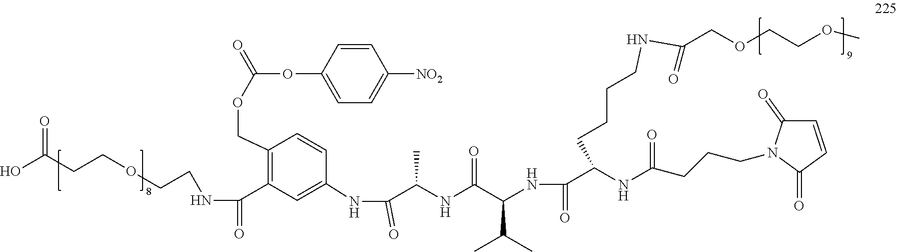

To a solution of compound 224 (703 mg, 0.403 mmol) in DCM (9 mL) was added TFA (3 mL). The mixture was stirred at room temperature for 1 h, and then concentrated under reduced pressure to give 225 (680 mg, 100% yield) as a colorless oil. ESI MS m/z: calcd for C₇₇H₁₂₁N₇O₃₄ [M+H]⁺: 1688.80; found 1687.49.

### Example 147: 1-(5-((37S,40S,43S)-37-(4-(2,5-dioxo-2,5-dihydro-1H-pyrrol-1-yl) butanamido)-40-isopropyl-43-methyl-31,38,41-trioxo-2,5,8,11,14,17,20,23,26,29-decaoxa-32,39,42-triazatetratetracontan-44-amido)-2-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)-1-oxo-5,8,11,14,17,20,23,26-octaoxa-2-azanonacosan-29-oic acid (227)

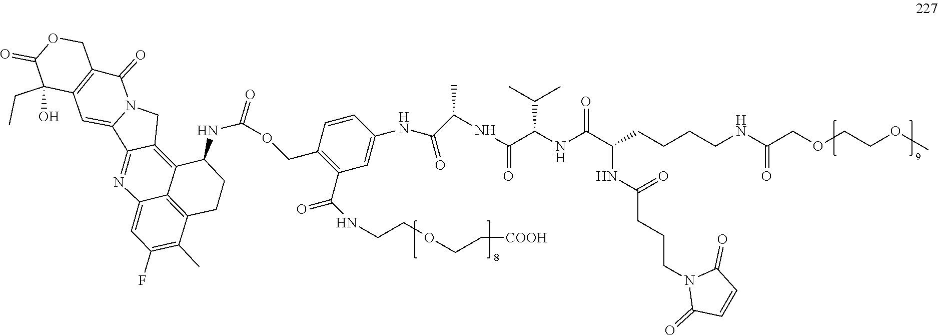

To a solution of compound 225 (680 mg, 0.403 mmol), Exatecan (214 mg, 0.403 mmol) and HOBt (54 mg, 0.403 mmol) in DMF (10 mL) was added DIEA (208 mg, 1.612 mmol). The reaction mixture was stirred at room temperature for 3 h, and then concentrated under reduced pressure. The resulting residue was purified by prep-HPLC to give 227 (548 mg, 68% yield) as a yellow solid. ESI MS m/z: calcd for C₉₅H₁₃₉FN₁₀O₃₄ [M+H]⁺: 1983.94; found 1984.29.

### Example 148: 2,5-dioxopyrrolidin-1-yl 1-(5-((37S,40S,43S)-37-(4-(2,5-dioxo-2,5-dihydro-1H-pyrrol-1-yl) butanamido)-40-isopropyl-43-methyl-31,38,41-trioxo-2,5,8,11,14,17,20,23,26,29-decaoxa-32,39,42-triazatetratetracontan-44-amido)-2-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)-1-oxo-5,8,11,14,17,20,23,26-octaoxa-2-azanonacosan-29-oate (228)

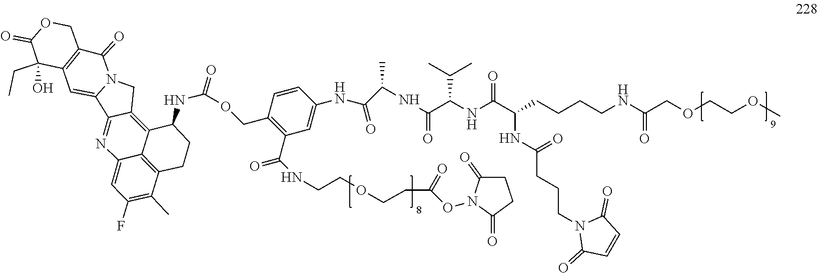

To a solution of compound 227 (150 mg, 0.076 mmol) and NHS (11 mg, 0.091 mmol) in DCM (3 mL) was added EDCI (22 mg, 0.113 mmol). The reaction mixture was stirred at room temperature for 2 h, and then concentrated under reduced pressure to give 228 (160 mg, 100% yield) as a yellow oil. ESI MS m/z: calcd for C₉₉H₁₄₂FN₁₁O₃₆ [M+H]⁺: 2080.96; found 2081.13.

### Example 149: (2S,5S,8S,11S, 14S,17S,20S)-2,5-bis(2-amino-2-oxoethyl)-17-(3-amino-3-oxopropyl)-11,14-di((S)-sec-butyl)-20-(1-(5-((37S,40S,43S)-37-(4-(2,5-dioxo-2,5-dihydro-1H-pyrrol-1-yl) butanamido)-40-isopropyl-43-methyl-31,38,41-trioxo-2,5,8,11,14,17,20,23,26,29-decaoxa-32,39,42-triazatetratetracontan-44-amido)-2-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)-1-oxo-5,8,11,14,17,20,23,26-octaoxa-2-azanonacosan-29-amido)-8-methyl-4,7,10,13,16,19-hexaoxo-3,6,9,12,15,18-hexaazadocosanedioic acid (229)

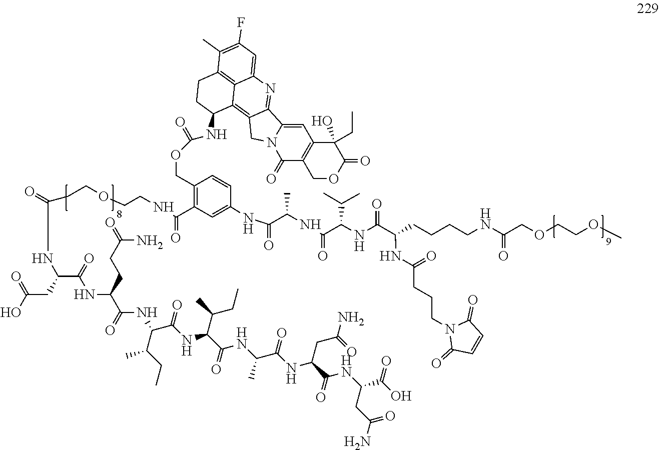

To a solution of compound 228 (129 mg, 0.080 mmol) and compound 222 (63 mg, 0.080 mmol) in DMF (3 mL) was added DIEA (21 mg, 0.160 mmol). The reaction mixture was stirred at room temperature for 1 h, and then concentrated under reduced pressure. The resulting residue was purified by prep-HPLC to give 229 (130 mg, 72% yield) as white solid. ESI MS m/z: calcd for C₁₀₂H₁₇₁N₁₇O₄₁ [M+2H]²⁺: 1375.67; found 1375.71.

### Example 150: 2-(hydroxymethyl)-N-(3-methyl-23-(11-oxidaneyl)-313,6,9,12,15,18,21-heptaoxatricosyl)-5-nitrobenzamide (233)

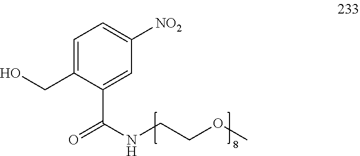

To a solution of compound 231 (187 mg, 1.043 mmol) in DMF (2 mL) was added compound 232 (200 mg, 0.522 mmol). The mixture was stirred at RT for 24 h and extracted with DCM/MeOH (v/v=19/1) (10 mL×3). The combined organic layers were washed with H₂O (10 mL×3), dried over anhydrous Na₂SO₄ and concentrated in vacuo. The residue was purified by silica gel column chromatography (eluted with DCM/MeOH=19/1) to afford 233 (130 mg, 45% yield) as a light yellow oil. ESI: m/z: calcd for C₂₅H₄₃N₂O₁₂ [M+H]⁺: 563.3; found 563.21.

### Example 151: 5-amino-2-(hydroxymethyl)-N-(3-methyl-23-(11-oxidaneyl)-313,6,9,12,15,18,21-heptaoxatricosyl)benzamide (234)

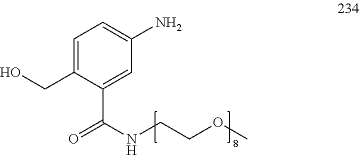

To a solution of compound 233 (52.4 g, 93.137 mmol) in MeOH (250 mL) was added Pd/C (5.24 g, 10 wt %). The mixture was stirred at RT for 3 h under H₂ and then filtered over Celite. The filtrate was concentrated in vacuo to afford 234 (49.61 g, crude) as a yellow oil. ESI: m/z: calcd for C₂₅H₄₅N₂O₁₀ [M+H]⁺: 533.34; found 533.83.

### Example 152: (9H-fluoren-9-yl)methyl(S)-(1-((4-(hydroxymethyl)-3-((3-methyl-23-(11-oxidaneyl)-313,6,9,12,15,18,21-heptaoxatricosyl)carbamoyl)phenyl)amino)-1-oxopropan-2-yl) carbamate (236)

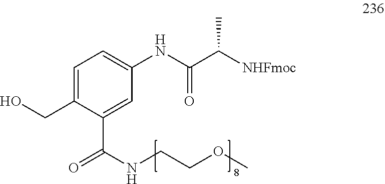

To a solution of compound 234 (49.61 g, 93.142 mmol) and compound 35 (37.7 g, 121.084 mmol) in THF (250 mL) was added EEDQ (29.94 g, 121.084 mmol). The mixture was stirred at RT overnight and then concentrated in vacuo. The residue was purified by silica gel column chromatography (eluted with DCM/MeOH=20/1) to afford 236 (66.8 g, 87% yield in two steps) as a light yellow oil. ESI: m/z: calcd for C₄₃H₆₀N₃O₁₃ [M+H]⁺: 826.41; found 826.88.

### Example 153: (S)-5-(2-aminopropanamido)-2-(hydroxymethyl)-N-(3-methyl-23-(11-oxidaneyl)-313,6,9,12,15,18,21-heptaoxatricosyl)benzamide (237)

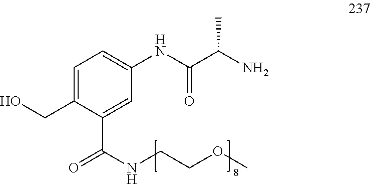

To a solution of compound 236 (1.00 g, 1.211 mmol) in DMF (50 mL) was added piperidine (150 mg, 1.816 mmol). The mixture was stirred at RT for 0.5 h, and then concentrated in vacuo to afford 237 (1.05 g, crude) as a white solid. ESI: m/z: calcd for C₂₈H₅₀N₃O₁₁ [M+H]⁺: 604.34; found 605.25.

### Example 154: (9H-fluoren-9-yl)methyl((S)-1-(((S)-1-((3-((2,5,8,11,14,17,20,23-octaoxapentacosan-25-yl)carbamoyl)-4-(hydroxymethyl)phenyl)amino)-1-oxopropan-2-yl)amino)-3-methyl-1-oxobutan-2-yl)carbamate (239)

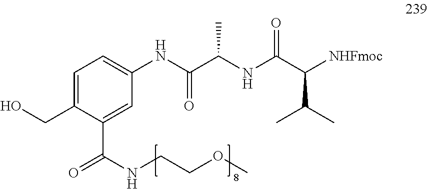

To a solution of compound 237 (1.05 g, 1.739 mmol) and compound 38 (0.89 g, 2.609 mmol) in DMF (10 mL) were added HATU (991 mg, 2.609 mmol) and DIPEA (450 mg, 3.478 mmol). The mixture was stirred at RT for 10 min and concentrated in vacuo. The residue was purified by silica gel column chromatography (eluted with DCM/MeOH=50/1) to afford 239 (1.28 g, 80% yield) as a yellow oil. ESI: m/z: calcd for C₄₈H₆₉N₄O₁₄ [M+H]⁺: 925.57; found 925.76.

### Example 155: (9H-fluoren-9-yl)methyl((S)-1-(((S)-1-((3-((2,5,8,11,14,17,20,23-octaoxapentacosan-25-yl)carbamoyl)-4-((((4-nitrophenoxy)carbonyl)oxy)methyl)phenyl)amino)-1-oxopropan-2-yl)amino)-3-methyl-1-oxobutan-2-yl)carbamate (241)

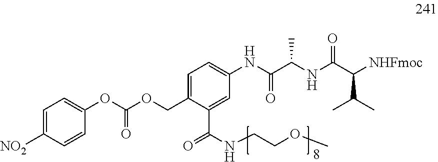

To a solution of compound 239 (375 mg, 0.405 mmol) in DCM (5 mL) was added compound 240 (246 mg, 0.810 mmol), then added DIEPA (375 mg, 0.405 mmol). The mixture was stirred at RT overnight and used for the next step without work-up. ESI: m/z: calcd for C₅₅H₇₂N₅O₁₈ [M+H]⁺: 1090.58; found 1090.87.

### Example 156: 2-((2,5,8,11,14,17,20,23-octaoxapentacosan-25-yl)carbamoyl)-4-((S)-2-((S)-2-((((9H-fluoren-9-yl)methoxy)carbonyl)amino)-3-methylbutanamido)propanamido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (243)

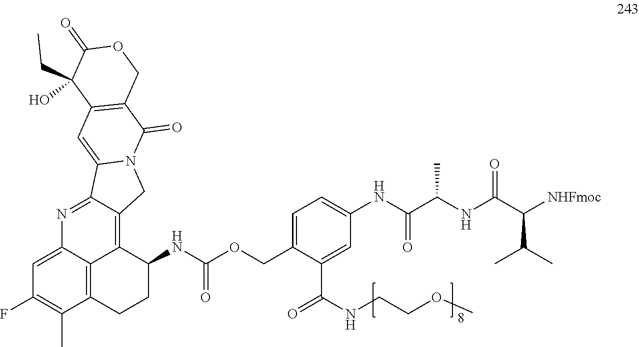

To a solution of previous step (241, 441 mg, 0.405 mmol) in DMF (5 mL) were added compound 242 (200 mg, 0.37 mmol) and HOBT (54 mg, 0.405 mmol). The mixture was stirred at RT for 1 h and concentrated in vacuo. The residue was purified by prep-HPLC (eluted with CH₃CN/H₂O=65/35) to afford 243 (154 mg, 30% yield in two steps) as a yellow oil. ESI: m/z: calcd for C₇₃H₈₉FN₇O₁₉ [M+H]⁺: 1386.64; found 1387.45.

### Example 157: 2-((2,5,8,11,14,17,20,23-octaoxapentacosan-25-yl)carbamoyl)-4-((S)-2-((S)-2-amino-3-methylbutanamido)propanamido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]Indolizino[1,2-b]quinolin-1-yl)carbamate (244)

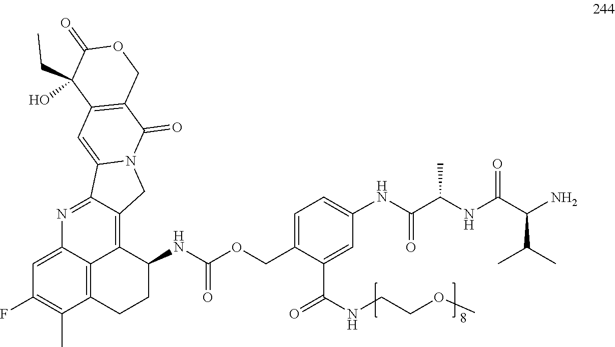

To a solution of compound 243 (154 mg, 0.111 mmol) in DMF (2 mL) was added piperidine (14 mg, 0.167 mmol). The mixture was stirred at RT for 0.5 h, and then concentrated in vacuo to afford 244 (129 mg, crude) as a white solid. ESI: m/z: calcd for C₅₈H₇₉FN₇O₁₇ [M+H]⁺: 1164.52; found 1164.32.

### Example 158: benzyl(S)-5-(((S)-1-(((S)-1-((3-((2,5,8,11,14,17,20,23-octaoxapentacosan-25-yl) carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (246)

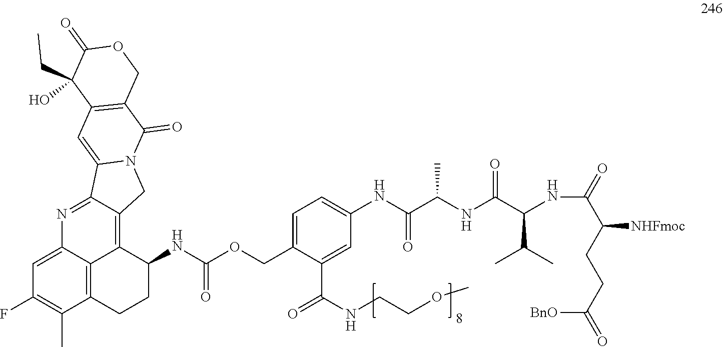

To a solution of compound 244 (129 mg, 0.111 mmol) and compound 245 (66 mg, 0.144 mmol) in DMF (2 mL) were added HATU (55 mg, 0.144 mmol) and DIPEA (29 mg, 0.222 mmol). The mixture was stirred at RT for 10 min and concentrated in vacuo. The residue was purified by silica gel column chromatography (eluted with DCM/MeOH=20/1) to afford 246 (152 mg, 85% yield in two steps) as a yellow solid. ESI: m/z: calcd for C₈₅H₁₀₂FN₈O₂₂ [M+H]⁺: 1605.71; found 1605.52.

### Example 159: (S)-5-(((S)-1-(((S)-1-((3-((2,5,8,11,14,17,20,23-octaoxapentacosan-25-yl) carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoic acid (247)

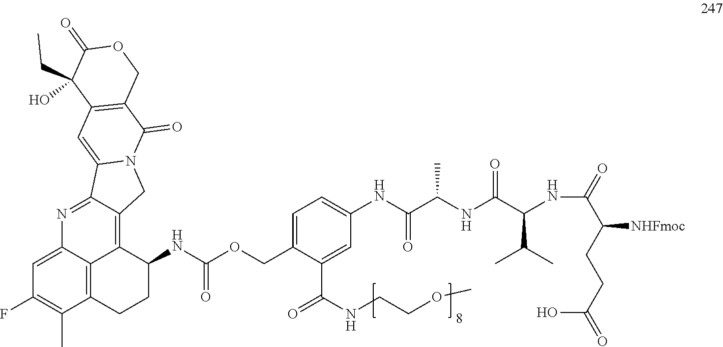

To a solution of compound 246 (152 mg, 0.095 mmol) in MeOH (5 mL) was added Pd/C (15 mg, 10% Pd). The mixture was stirred at RT under H₂ overnight and then filtered with Celite. The filtrate was concentrated in vacuo to afford 247 (138 mg, 96% yield) as a white solid. ESI: m/z: calcd for C₇₈H₉₆FN₈O₂₂ [M+H]⁺: 1515.71; found 1515.32.

### Example 160: allyl((S)-1-(((S)-1-((3-((2,5,8,11,14,17,20,23-octaoxapentacosan-25-yl) carbamoyl)-4-(hydroxymethyl)phenyl)amino)-1-oxopropan-2-yl)amino)-3-methyl-1-oxobutan-2-yl) carbamate (249)

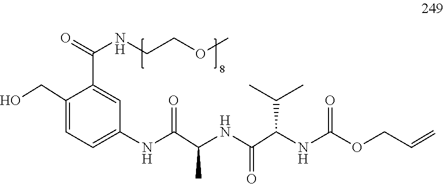

To a solution of compound 237 (730 mg, 1.211 mmol) and compound 248 (365 mg, 1.816 mmol) in DMF (10 mL) were added HATU (691 mg, 1.816 mmol) and DIPEA (313 mg, 2.421 mmol). The mixture was stirred at RT for 10 min and concentrated in vacuo. The residue was purified by silica gel column chromatography (eluted with DCM/MeOH=20/1) to afford 249 (851 mg, 90% yield in two steps) as a white solid. ESI: m/z: calcd for C₃₇H₆₃N₄O₁₄ [M+H]⁺: 787.44; found 787.41.

### Example 161: allyl((S)-1-(((S)-1-((3-((2,5,8,11,14,17,20,23-octaoxapentacosan-25-yl) carbamoyl)-4-((((4-nitrophenoxy)carbonyl)oxy)methyl)phenyl)amino)-1-oxopropan-2-yl)amino)-3-methyl-1-oxobutan-2-yl)carbamate (250)

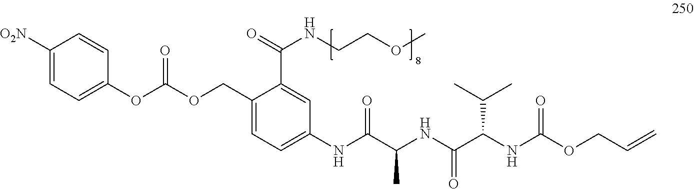

To a solution of compound 249 (300 mg, 0.381 mmol) in DCM (5 mL) were added compound 240 (232 mg, 0.762 mmol) and DIPEA (99 mg, 0.762 mmol). The mixture was stirred at RT overnight and then concentrated in vacuo. The residue was prep-HPLC (eluted with CH₃CN/H₂O=65/35) to afford 250 (246 mg, 68% yield) as a yellow oil. ESI: m/z: calcd for C₄₄H₆₆N₅O₁₈ [M+H]⁺: 952.44; found 952.42.

### Example 162: 2-((2,5,8,11,14,17,20,23-octaoxapentacosan-25-yl)carbamoyl)-4-((S)-2-((S)-2-(((allyloxy)carbonyl)amino)-3-methylbutanamido)propanamido)benzyl (1-((2R,4R,5R)-4-((tert-butyldimethylsilyl)oxy)-5-(((tert-butyldimethylsilyl)oxy)methyl)-3,3-difluorotetrahydrofuran-2-yl)-2-oxo-1,2-dihydropyrimidin-4-yl)carbamate (252)

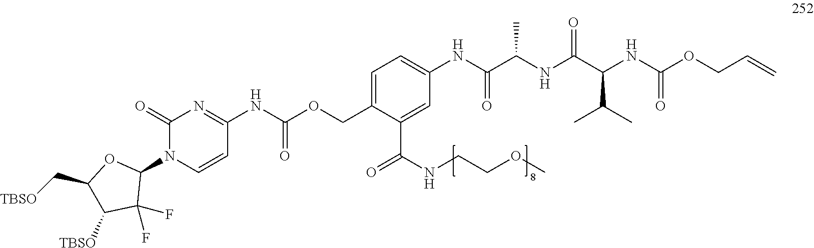

To a solution of compound 250 (152 mg, 0.31 mmol) in THF (5 mL) was added LiHMDS (1 mol/L in THF) (0.388 mL, 0.388 mmol) at −60° C. dropwise. The mixture was warmed to RT for 0.5 h. Compound 251 (246 mg, 0.258 mmol) was added and the reaction mixture was stirred for 0.5 h, quenched with saturated NH₄Cl solution, then extracted with DCM (10 mL×3). The combined organic layers were dried over anhydrous Na₂SO₄ and concentrated in vacuo to afford 252 (336 mg, crude) as a yellow oil. ESI: m/z: calcd for C₅₉H₁₀₀F₂N₇O₁₉Si₂ [M+H]⁺: 1304.72; found 1304.62.

### Example 163: 2-((2,5,8,11,14,17,20,23-octaoxapentacosan-25-yl)carbamoyl)-4-((S)-2-((S)-2-amino-3-methylbutanamido)propanamido)benzyl (1-((2R,4R,5R)-4-((tert-butyldimethylsilyl)oxy)-5-(((tert-butyldimethylsilyl)oxy)methyl)-3,3-difluorotetrahydrofuran-2-yl)-2-oxo-1,2-dihydropyrimidin-4-yl)carbamate (253)

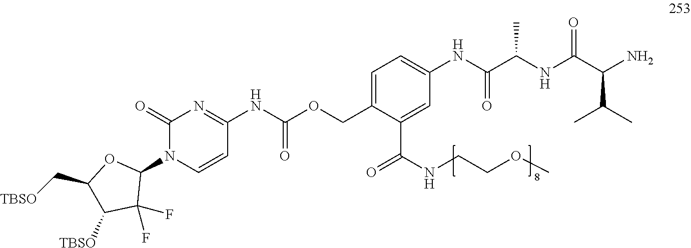

To a solution of compound 252 (336 mg, 0.258 mmol) in DCM (5 mL) were added Pd(PPh₃)₄ (11 mg, 0.010 mmol) and pyrrolidine (36 mg, 0.516 mmol). The mixture was stirred at RT for 0.5 h, and then concentrated in vacuo to afford 253 (315 mg, crude) as a yellow oil. ESI: m/z: calcd for C₅₅H₉₆F₂N₇O₁₇Si2 [M+H]⁺: 1220.61; found 1220.40.

### Example 164: 2-((2,5,8, 11,14,17,20,23-octaoxapentacosan-25-yl)carbamoyl)-4-((S)-2-((S)-2-amino-3-methylbutanamido)propanamido)benzyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (254)

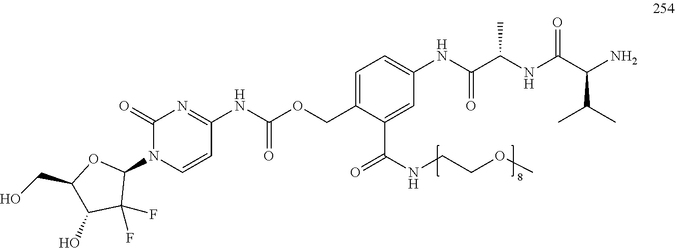

To a solution of compound 253 (315 mg, 0.258 mmol) in THF (5 mL) was added TBAF (1 mol/L in THF) (0.516 mL, 0.516 mmol). The mixture was stirred at RT for 0.5 h, and then concentrated in vacuo. The residue was purified by column chromatography on silica (eluted with DCM/MeOH=10/1) to afford 254 (112 mg, 44% yield in three steps) as a yellow foam. ESI: m/z: calcd for C₄₃H₆₈F₂N₇O₁₇ [M+H]⁺: 992.50; found 992.30.

### Example 165: 2-((2,5,8,11,14,17,20,23-octaoxapentacosan-25-yl)carbamoyl)-4-((2S,5S,8S, 13S, 16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-((((9H-fluoren-9-yl)methoxy)carbonyl)amino)-5,13-diisopropyl-2,16-dimethyl-4,7,11,14,17-pentaoxo-3,6,12,15-tetraazaheptadecanamido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (255)

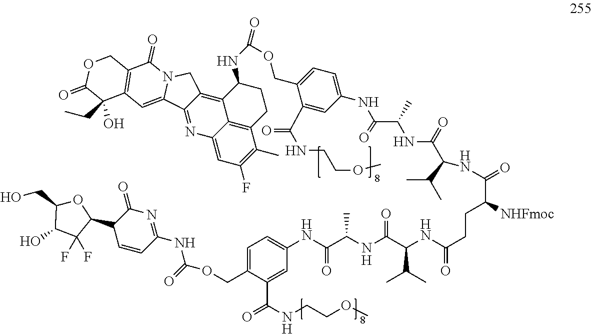

To a solution of compound 254 (138 mg, 0.091 mmol) and compound 247 (90 mg, 0.091 mmol) in DMF (2 mL) were added HATU (42 mg, 0.109 mmol) and DIPEA (24 mg, 0.182 mmol). The mixture was stirred at RT for 10 min and purified by column chromatography on silica (eluted with DCM/MeOH=7/1) to afford 255 (171 mg, 75% yield) as a yellow solid. ESI: m/z: calcd for C₁₂₁H₁₆₁F₃N₁₅O₃₈ [M+H]⁺: 2489.10; found 2490.00.

### Example 166: 2-((2,5,8,11,14,17,20,23-octaoxapentacosan-25-yl)carbamoyl)-4-((2S,5S,8S,13S, 16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-amino-5,13-diisopropyl-2,16-dimethyl-4,7,11,14,17-pentaoxo-3,6,12,15-tetraazaheptadecanamido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (256)

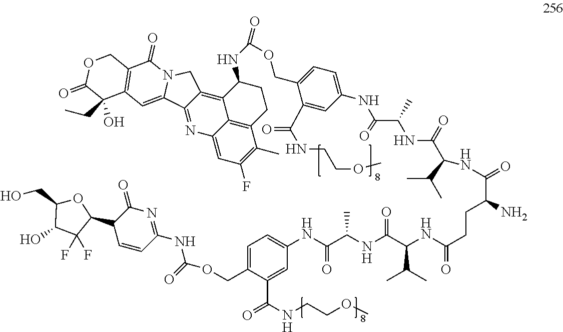

To a solution of compound 255 (171 mg, 0.069 mmol) in DMF (2 mL) was added piperidine (9 mg, 0.103 mmol). The mixture was stirred at RT for 1 h, purified by silica gel column chromatography (eluted with DCM/MeOH=5/1˜2/1) to afford 256 (124 mg, 80% yield) as a yellow solid. ESI: m/z: calcd for C₁₀₆H₁₅₁F₃N₁₅O₃₆ [M+H]⁺: 2267.02; found 2268.10.

### Example 167: ((((2S,5S,8S,25S,28S,31S)-8,25-bis(3-(((S)-1-(((S)-1-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-1-oxopropan-2-yl)amino)-3-methyl-1-oxobutan-2-yl)amino)-3-oxopropyl)-16,17-bis(2,5-dioxo-2,5-dihydro-1H-pyrrol-1-yl)-5,28-diisopropyl-2,31-dimethyl-4,7,10,15,18,23,26,29-octaoxo-3,6,9,14,19,24,27,30-octaazadotriacontanedioyl)bis(azanediyl))bis(2-((2,5,8,11,14,17,20,23-octaoxapentacosan-25-yl)carbamoyl)-4,1-phenylene))bis(methylene)bis(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate) (258)

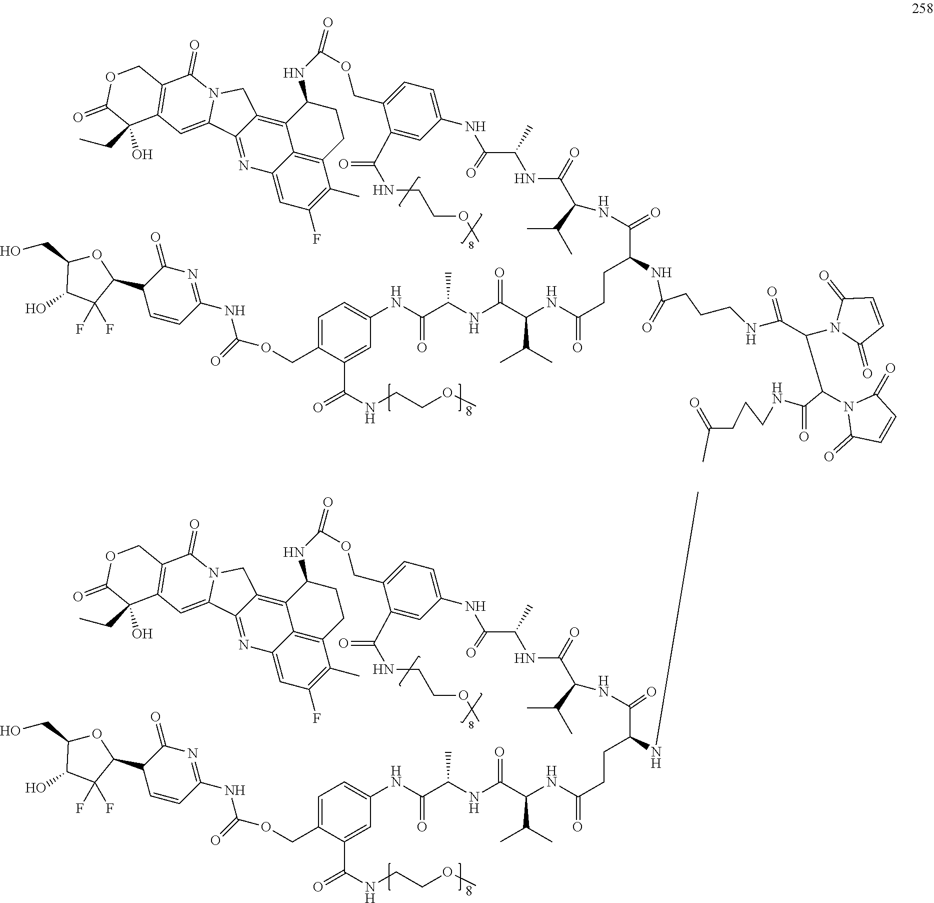

To a solution of compound 256 (56 mg, 0.025 mmol) and compound 257 (10 mg, 0.012 mmol) in DMF (2 mL) was added DIPEA (4 mg, 0.030 mmol). The mixture was stirred at RT for 0.5 h and purified by prep-HPLC (eluted with CH₃CN/H₂O (containing 0.01% formic acid)=50/50) to afford 258 (26 mg, 42% yield) as a white solid. ESI: m/z: calcd for C₂₃₂H₃₁₉F₆N₃₄O₈₀ [M+2H]²⁺: 2488.09; found 2488.70.

### Example 168: 2-((2,5,8,11, 14, 17,20,23-octaoxapentacosan-25-yl)carbamoyl)-4-((37S,45S,48S,51S)-45-(3-(((S)-1-(((S)-1-((3-((2,5,8,11,14,17,20,23-octaoxapentacosan-25-yl) carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-1-oxopropan-2-yl)amino)-3-methyl-1-oxobutan-2-yl)amino)-3-oxopropyl)-37-(4-(2,5-dioxo-2,5-dihydro-1H-pyrrol-1-yl) butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (261)

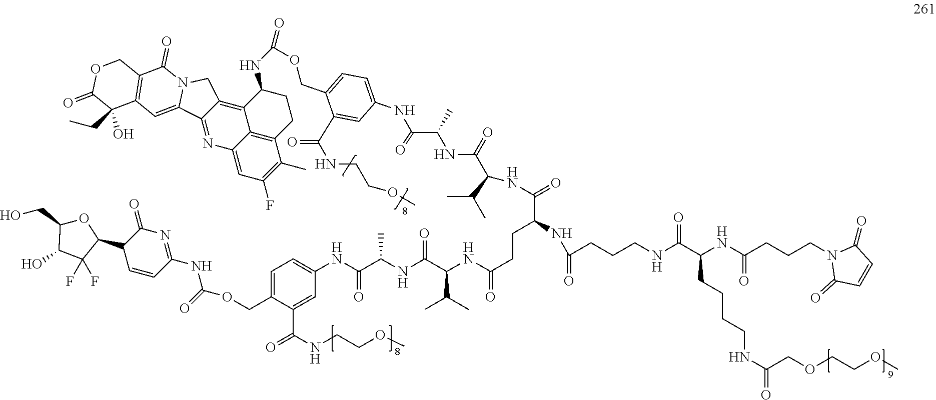

To a solution of compound 256 (66 mg, 0.029 mmol) and compound 260 (25 mg, 0.029 mmol) in DMF (2 mL) were added HATU (13 mg, 0.035 mmol) and DIPEA (8 mg, 0.058 mmol). The mixture was stirred at RT for 10 min and purified by prep-HPLC (eluted with CH₃CN/H₂O (containing 0.01% formic acid)=45/55) to afford 261 (50 mg, 55% yield) as a white solid. ESI: m/z: calcd for C₁₄₅H₂₁₇F₃N₁₉O₅₂ [M+2H]²⁺: 1557.24; found 1557.15.

### Example 169: 5-((S)-2-((S)-2-amino-3-methylbutanamido)propanamido)-2-(hydroxymethyl)-N-(3-methyl-23-(11-oxidaneyl)-313,6,9,12,15, 18,21-heptaoxatricosyl)benzamide (263)

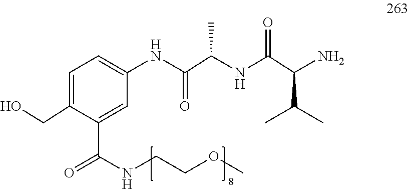

To a solution of compound 239 (1.46 g, 1.578 mmol) in DMF (15 mL) was added piperidine (1.5 mL). The mixture was stirred at room temperature for 10 min and concentrated under reduced pressure to give 263 (1.11 g, 100% yield) as a white solid. ESI: m/z: calcd for C₃₃H₅₈N₄O₁₂ [M+H]⁺: 703.41; found 703.26.

### Example 170: tert-butyl(S)-4-(((benzyloxy)carbonyl)amino)-5-(((S)-1-(((S)-1-((4-(hydroxymethyl)-3-((3-methyl-23-(11-oxidaneyl)-313,6,9,12,15,18,21-heptaoxatricosyl)carbamoyl)phenyl)amino)-1-oxopropan-2-yl)amino)-3-methyl-1-oxobutan-2-yl)amino)-5-oxopentanoate (265)

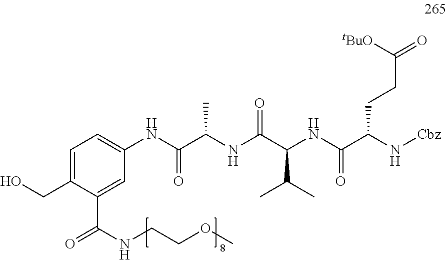

To a solution of compound 263 (1.11 g, 1.579 mmol), compound 264 (0.87 g, 1.895 mmol) and HATU (0.66 g, 1.737 mmol) in DMF (10 mL) was added DIEA (0.41 g, 3.158 mmol). The reaction mixture was stirred at room temperature for 1.5 h, and then concentrated under reduced pressure. The resulting residue was purified by flash column chromatography to give 265 (1.81 g, 100% yield) as light yellow oil. ESI: m/z: calcd for C₅₀H₇₉N₅O₁₇ [M+H]⁺: 1022.55; found 1022.61.

### Example 171: tert-butyl(S)-4-(((benzyloxy)carbonyl)amino)-5-(((S)-1-(((S)-1-((4-(hydroxymethyl)-3-((3-methyl-23-(11-oxidaneyl)-313,6,9,12,15, 18,21-heptaoxatricosyl)carbamoyl)phenyl)amino)-1-oxopropan-2-yl)amino)-3-methyl-1-oxobutan-2-yl)amino)-5-oxopentanoate (267)

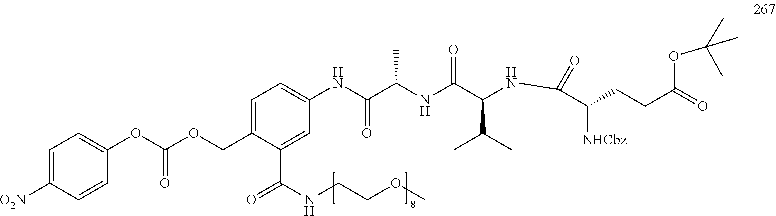

To a solution of compound 265 (1.72 g, 1.503 mmol) and compound 266 (0.61 g, 3.006 mmol) in DCM (20 mL) were added DMAP (0.18 g, 1.503 mmol) and DIEA (0.49 g, 3.758 mmol) at room temperature. The mixture was stirred at room temperature for 1.0 h, washed with brine (5 mL×2), then concentrated under reduced pressure to give 267 (1.97 g, 100% yield) as a yellow oil. ESI: m/z: calcd for C₅₇H₈₂N₆O₂₁ [M+H]⁺: 1187.55; found 1187.42.

### Example 172: (S)-4-(((benzyloxy)carbonyl)amino)-5-(((S)-3-methyl-1-(((S)-1-((3-((3-methyl-23-(11-oxidaneyl)-313,6,9,12, 15,18,21-heptaoxatricosyl)carbamoyl)-4-((((4-nitrophenoxy)carbonyl)oxy) methyl)phenyl)amino)-1-oxopropan-2-yl)amino)-1-oxobutan-2-yl)amino)-5-oxopentanoic acid (268)

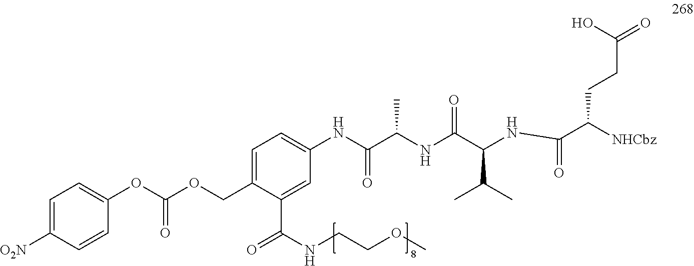

To a solution of compound 267 (1.83 g, 1.541 mmol) in DCM (10 mL) was added TFA (10 mL). The mixture was stirred at room temperature for 1 h, and then concentrated under reduced pressure. The resulting residue was purified by prep-HPLC to give 268 (1.69 g, 100% yield) as a white solid. ESI: m/z: calcd for C₅₃H₇₄N₆O₂₁ [M+H]⁺: 1131.49; found 1131.50.

### Example 173: perfluorophenyl(S)-4-(((benzyloxy)carbonyl)amino)-5-(((S)-3-methyl-1-(((S)-1-((3-((3-methyl-23-(11-oxidaneyl)-313,6,9,12,15,18,21-heptaoxatricosyl)carbamoyl)-4-((((4-nitrophenoxy) carbonyl)oxy)methyl)phenyl)amino)-1-oxopropan-2-yl)amino)-1-oxobutan-2-yl)amino)-5-oxopentanoate (269)

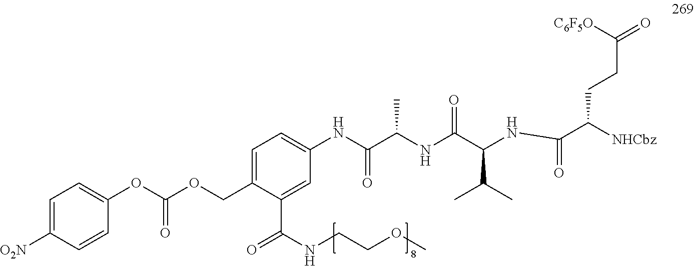

To a solution of compound 268 (1.69 g, 1.494 mmol) and PFP—OH (330 mg, 1.793 mmol) in DCM (20 mL) was added EDCI (430 mg, 2.241 mmol) at room temperature. The mixture was stirred at room temperature for 1.0 h, washed with brine (10 mL×2), then concentrated under reduced pressure to give 269 (1.90 g, 97% yield) as a yellow oil. ESI: m/z: calcd for C₅₉H₇₃F₅N₆O₂₁ [M+H]⁺: 1297.47; found 1297.51.

### Example 174: benzyl ((2S,5S,8S)-1-((3-((2,5,8,11,14,17,20,23-octaoxapentacosan-25-yl)carbamoyl)-4-((((4-nitrophenoxy)carbonyl)oxy)methyl)phenyl)amino)-23-((1-((2R,4R,5R)-3,3-difluoro-4-hydroxy-5-(hydroxymethyl)tetrahydrofuran-2-yl)-2-oxo-1,2-dihydropyrimidin-4-yl)amino)-18,18-bis((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)-5-isopropyl-2-methyl-1,4,7,11,16,23-hexaoxo-20-oxa-3,6, 12,17-tetraazatricosan-8-yl)carbamate (271)

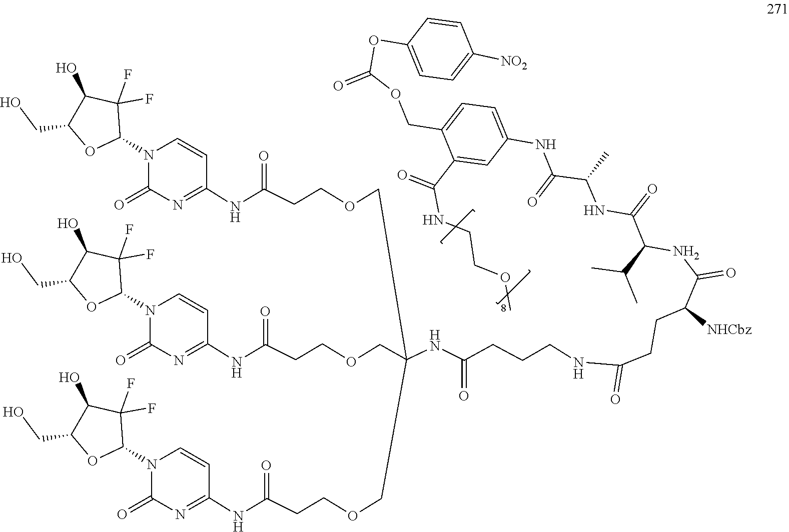

To a solution of compound 269 (99 mg, 0.079 mmol) and compound 270 (88 mg, 0.076 mmol) in DMF (2 mL) was added DIPEA (30 mg, 0.229 mmol). The mixture was stirred at RT for 0.5 h, and then concentrated in vacuo to afford 271 (179 mg, crude) as a yellow oil. ESI: m/z: calcd for C₉₇H₁₂₉F₆N₁₇O₃₉ [M+H]⁺: 2270.85; found 2270.46.

### Example 175:2-((2,5,8,11,14,17,20,23-octaoxapentacosan-25-yl)carbamoyl)-4-((2S,5S,8S)-8-(((benzyloxy)carbonyl)amino)-23-((1-((2R,4R,5R)-3,3-difluoro-4-hydroxy-5-(hydroxymethyl)tetrahydrofuran-2-yl)-2-oxo-1,2-dihydropyrimidin-4-yl)amino)-18,18-bis((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)-5-isopropyl-2-methyl-4,7,11,16,23-pentaoxo-20-oxa-3,6,12,17-tetraazatricosanamido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (272)

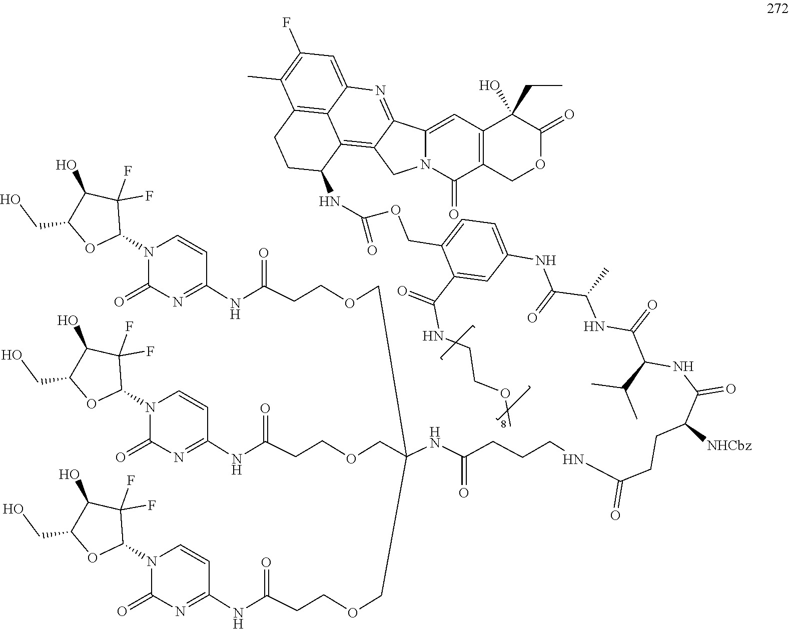

To a solution of compound 271 (179 mg, 0.079 mmol) in DMF (2 mL), Exatecan (40 mg, 0.076 mmol), HOBT (10 mg, 0.076 mmol) and DIPEA (20 mg, 0.152 mmol) were added. The reaction mixture was stirred at RT for 5 h and purified by prep-HPLC (eluted with CH₃CN/H₂O (containing 0.05% formic acid)=48/52) to afford 272 (98 mg, 51% yield in two steps) as a white solid. ESI: m/z: calcd for C₁₁₅H₁₄₇F₇N₁₉O₄₀ [M+2H]²⁺: 1284.50; found 1284.21.

### Example 176:2-((2,5,8,11,14,17,20,23-octaoxapentacosan-25-yl)carbamoyl)-4-((2S,5S,8S)-8-amino-23-((1-((2R,4R,5R)-3,3-difluoro-4-hydroxy-5-(hydroxymethyl)tetrahydrofuran-2-yl)-2-oxo-1,2-dihydropyrimidin-4-yl)amino)-18,18-bis((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl)-5-isopropyl-2-methyl-4,7,11,16,23-pentaoxo-20-oxa-3,6,12,17-tetraazatricosanamido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (273)

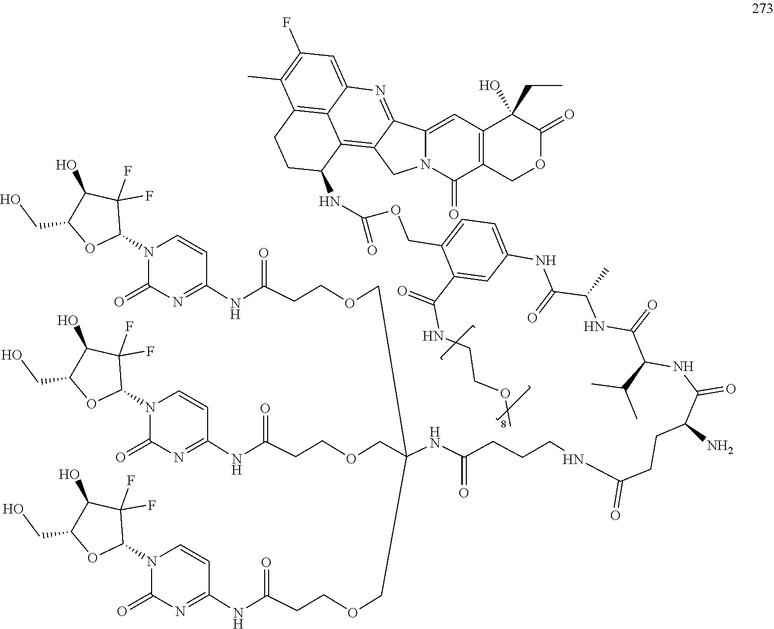

To a solution of compound 272 (98 mg, 0.038 mmol) in DMF (5 mL) was added Pd/C (10 mg, 10 wt %). The mixture was stirred at RT for 6 h under H₂ and then filtered over Celite. The filtrate was purified by prep-HPLC (eluted with CH₃CN/H₂O=40/60) to afford 273 (57 mg, 61% yield) as a white solid. ESI: m/z: calcd for C₁₀₇H₁₄₁F₇N₁₉O₃₈ [M+H]⁺: 2432.95; found 2432.17.

### Example 177: ((((2S,5S,8S,25S,28S,31S)-8,25-bis(3-((4-((1,3-bis(3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)-2-((3-((1-((2R,4R,5R)-3,3-difluoro-4-hydroxy-5-(hydroxymethyl)tetrahydrofuran-2-yl)-2-oxo-1,2-dihydropyrimidin-4-yl)amino)-3-oxopropoxy)methyl) propan-2-yl)amino)-4-oxobutyl)amino)-3-oxopropyl)-16,17-bis(2,5-dioxo-2,5-dihydro-1H-pyrrol-1-yl)-5,28-diisopropyl-2,31-dimethyl-4,7,10,15,18,23,26,29-octaoxo-3,6,9,14,19,24,27,30-octaazadotriacontanedioyl)bis(azanediyl))bis(2-((2,5,8,11,14,17,20,23-octaoxapentacosan-25-yl)carbamoyl)-4,1-phenylene))bis(methylene)bis(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate) (274)

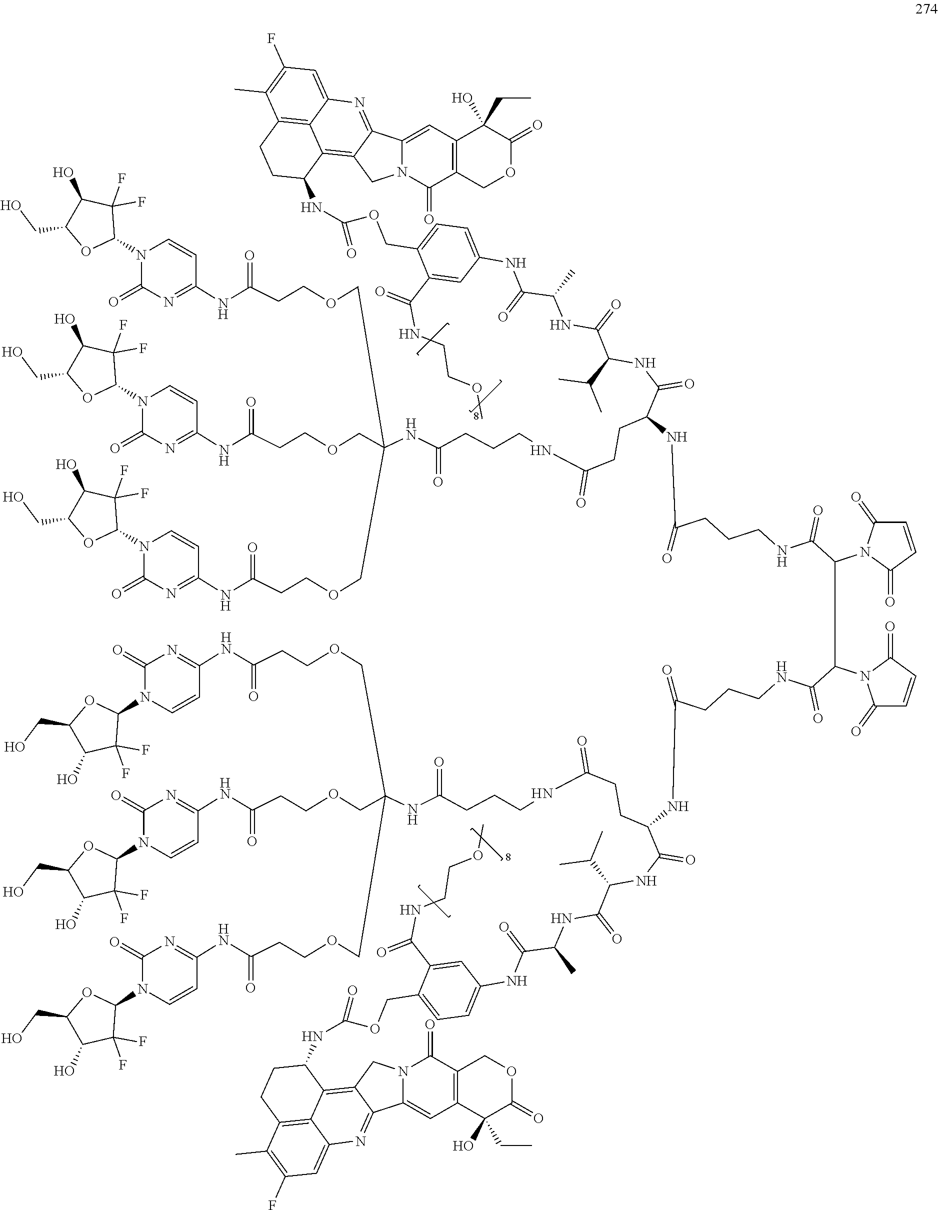

To a solution of compound 273 (54 mg, 0.022 mmol) and compound 258 (9 mg, 0.011 mmol) in DMF (2 mL) was added DIPEA (6 mg, 0.044 mmol). The mixture was stirred at RT for 2 h and purified by prep-HPLC (eluted with CH₃CN/H₂O (containing 0.05% formic acid)=42/58) to afford 274 (19 mg, 32% yield) as a white solid. ESI: m/z: calcd for C₂₃₄H₂₉₉F₁₄N₄₂O₈₄ [M+3H]³⁺: 1769.67; found 1770.61.

### Example 178: rac-tert-butyl ((benzyloxy)carbonyl)-D-alanylglycinate (278)

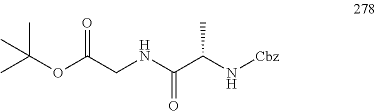 [SMILES: C[C@H](NC(=O)OCc1ccccc1)C(=O)NCC(=O)OC(C)(C)C]

To a solution of compound 277 (5.00 g, 22.398 mmol) and compound 276 (3.75 g, 22.398 mmol) and HATU (9.37 g, 24.638 mmol) in DCM (60 mL) was added DIEA (7.24 g, 55.996 mmol). The reaction mixture was stirred at room temperature for 1 h and quenched by H₂O (10 mL). The organic phase was separated then washed with saturated Na₂CO₃, dried over Na₂SO₄ and concentrated under reduced pressure. The resulting residue was purified by flash column chromatography to afford 278 (1.93 g, 87% yield) as a white solid. ESI: m/z: calcd for C₁₇H₂₅N₂O₅ [M+H]⁺: 337.17; found 337.11.

### Example 179: rac-((benzyloxy)carbonyl)-D-alanylglycine (279)

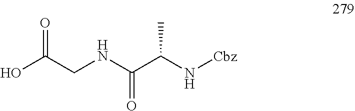 [SMILES: C[C@H](NC(=O)OCc1ccccc1)C(=O)NCC(=O)O]

To a solution of compound 278 (6.80 g, 20.215 mmol) in DCM (60 mL) was added TFA (35 mL). The mixture was stirred at room temperature for 16 h, and then concentrated under reduced pressure. The resulting residue was triturated with petroleum ether to afford 279 (5.67 g, 20.215 mmol, 100% yield) as a white gum. ESI: m/z: calcd for C₁₃H₁₇N₂O₅ [M+H]⁺: 281.11; found 281.12.

### Example 180: rac-(R)-(2 (((benzyloxy)carbonyl)amino)propanamido) methyl acetate (280)

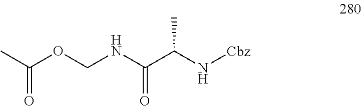 [SMILES: CC(=O)OCNC(=O)[C@H](C)NC(=O)OCc1ccccc1]

To a solution of compound 279 (2.84 g, 10.133 mmol) in THF (30 mL) were added lead tetraacetate (10.79 g, 10.133 mmol) and pyridine (0.80 g, 10.133 mmol). The mixture was stirred at 70° C. for 1 h before it was filtered through a pad of Celite. The filtrate was concentrated under reduced pressure. The resulting residue was purified by flash column chromatography to afford 280 (1.52 g, 51% yield) as a white solid. ESI: m/z: calcd for C₁₄H₁₈N₂O₅Na [M+Na]⁺: 317.11; found 316.82.

### Example 181: 3,3′-((2-(4-aminobutanamido)-2-((2-carboxyethoxy)methyl) propane-1,3-diyl)bis(oxy))dipropionic acid (282)

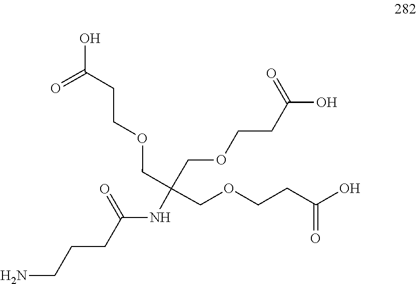 [SMILES: NCCCC(=O)NC(COCCC(=O)O)(COCCC(=O)O)COCCC(=O)O]

To a solution of compound 281 (5.00 g, 9.568 mmol) in DCM (50 mL) was added TFA (10 mL). The mixture was stirred at room temperature for 16 h, and then concentrated under reduced pressure. The resulting residue was washed with MTBE to afford 282 (4.04 g, 100% yield) as a colorless oil. ESI: m/z: calcd for C₁₇H₃₁N₂O₁₀ [M+H]⁺: 423.19; found 423.00.

### Example 182: 3,3′-((2-(4-aminobutanamido)-2-((2-carboxyethoxy)methyl) propane-1,3-diyl)bis(oxy))dipropionic acid (283)

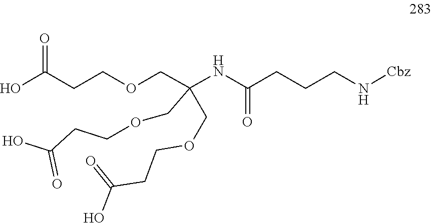 [SMILES: O=C(O)CCOCC(COCCC(=O)O)(COCCC(=O)O)NC(=O)CCCNC(=O)OCc1ccccc1]

To a solution of compound 282 (4.04 g, 9.564 mmol) in THF (40 mL) was added 1 N NaOH (50 mL). Then Cbz-Cl (6.52 g, 38.254 mmol) was added. The mixture was stirred at room temperature for 30 min before it was diluted with EA (30 mL). The aqueous phase was separated and adjusted pH to about 3 with 1 N HCl, concentrated and purified by prep-HPLC to afford 283 (1.50 g, 28% yield) as a white solid. ESI: m/z: calcd for C₂₅H₃₇N₂O₁₂ [M+H]⁺: 557.23; found 556.71.

### Example 183: rac-benzyl (1-((2R,4R,5R)-4-(((benzyloxy)carbonyl)oxy)-3,3-difluoro-5-((S)-6-methyl-5,8-dioxo-10-phenyl-2,9-dioxa-4,7-diazadecyl)tetrahydrofuran-2-yl)-2-oxo-1,2-dihydropyrimidin-4-yl)carbamate (285)

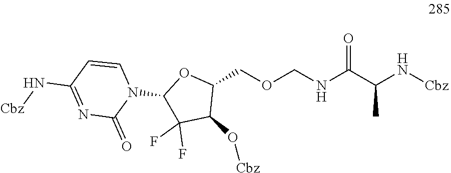 [SMILES: C[C@H](NC(=O)OCc1ccccc1)C(=O)NCOC[C@H]1O[C@@H](n2ccc(NC(=O)OCc3ccccc3)nc2=O)C(F)(F)[C@@H]1OC(=O)OCc1ccccc1]

To a solution of compound 284 (1.00 g, 1.882 mmol) and compound 280 (1.11 g, 3.763 mmol) in THF (10.0 mL) was added PPTS (0.19 g, 0.753 mmol). The reaction mixture was stirred at room temperature for 5 h, and then concentrated under reduced pressure. The resulting residue was purified by flash column chromatography to afford 285 (872 mg, 60% yield) as white foam. ESI MS m/z: calcd for C₃₇H₃₈F₂N₅O₁₁ [M+H]⁺: 766.25; found 765.61.

### Example 184: rac-(R)-2-amino-N-((((2S,3S,5S)-5-(4-amino-2-oxopyrimidin-1 (2H)-yl)-4,4-difluoro-3-hydroxytetrahydrofuran-2-yl)methoxy)methyl) propanamide (286)

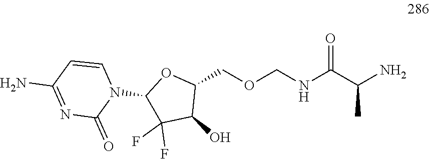 [SMILES: C[C@H](N)C(=O)NCOC[C@H]1O[C@@H](n2ccc(N)nc2=O)C(F)(F)[C@@H]1O]

To a solution of compound 285 (872 mg, 1.139 mmol) in MeOH (20 mL) at room temperature was added Pd/C (90 mg, 10 wt %). The resulting mixture was stirred at room temperature under a H₂ balloon for 1 h before it was filtered through a pad of Celite. The filtrate was concentrated under reduced pressure to afford 286 (413 mg, 99% yield) as a colorless oil. ESI MS m/z: calcd for C₁₃H₂₀F₂N₅O₅ [M+H]⁺: 364.14; found 363.81.

### Example 185: rac-benzyl ((R)-1-(((R)-1-(((((2S,3S,5S)-5-(4-amino-2-oxopyrimidin-1 (2H)-yl)-4,4-difluoro-3-hydroxytetrahydrofuran-2-yl)methoxy)methyl)amino)-1-oxopropan-2-yl)amino)-1-oxopropan-2-yl)carbamate (287)

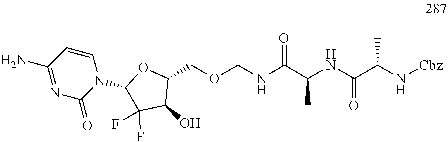 [SMILES: C[C@H](NC(=O)OCc1ccccc1)C(=O)N[C@@H](C)C(=O)NCOC[C@H]1O[C@@H](n2ccc(N)nc2=O)C(F)(F)[C@@H]1O]

To a solution of compound 286 (413 mg, 1.137 mmol) and compound 277 (254 mg, 1.137 mmol) and HATU (454 mg, 1.194 mmol) in DMF (5 mL) was added DIEA (220.4 mg, 1.705 mmol). The reaction mixture was stirred at room temperature for 4 h and then concentrated under reduced pressure. The resulting residue was purified by flash column chromatography to afford 287 (445 mg, 68% yield) as light yellow oil. ESI MS m/z: calcd for C₂₄H₃₁F₂N₆O₈ [M+H]⁺: 569.21; found 568.81.

### Example 186: rac-(R)-2-amino-N—((R)-1-(((((2S,3S,5S)-5-(4-amino-2-oxopyrimidin-1 (2H)-yl)-4,4-difluoro-3-hydroxytetrahydrofuran-2-yl)methoxy)methyl)amino)-1-oxopropan-2-yl) propanamide (288)

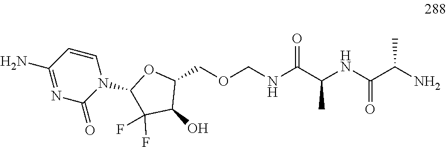 [SMILES: C[C@H](N)C(=O)N[C@@H](C)C(=O)NCOC[C@H]1O[C@@H](n2ccc(N)nc2=O)C(F)(F)[C@@H]1O]

To a solution of compound 287 (445 mg, 0.783 mmol) in MeOH (10 mL) at room temperature was added Pd/C (45 mg, 10 wt %). The resulting mixture was stirred at room temperature under a H₂ balloon for 7 h before it was filtered through a pad of Celite. The filtrate was concentrated under reduced pressure to afford 288 (340 mg, 100% yield) as a colorless oil. ESI MS m/z: calcd for C₁₆H₂₅F₂N₆O₆ [M+H]⁺: 435.17; found 434.81.

### Example 187: rac-benzyl ((6R,9R)-1-((2S,3S,5S)-5-(4-amino-2-oxopyrimidin-1 (2H)-yl)-4,4-difluoro-3-hydroxytetrahydrofuran-2-yl)-16,16-bis((6R,9R)-1-((2S,3S,5S)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18-tetraoxo-2,14-dioxa-4,7,10,17-tetraazahenicosan-21-yl) carbamate (289)

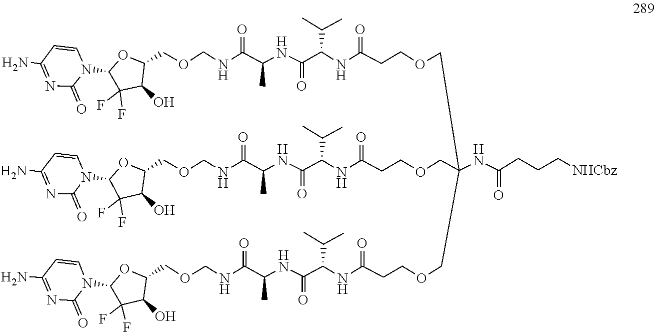 [SMILES: CC(C)[C@H](NC(=O)CCOCC(COCCC(=O)N[C@H](C(=O)N[C@@H](C)C(=O)NCOC[C@H]1O[C@@H](n2ccc(N)nc2=O)C(F)(F)[C@@H]1O)C(C)C)(COCCC(=O)N[C@H](C(=O)N[C@@H](C)C(=O)NCOC[C@H]1O[C@@H](n2ccc(N)nc2=O)C(F)(F)[C@@H]1O)C(C)C)NC(=O)CCCNC(=O)OCc1ccccc1)C(=O)N[C@@H](C)C(=O)NCOC[C@H]1O[C@@H](n2ccc(N)nc2=O)C(F)(F)[C@@H]1O]

To a solution of compound 288 (128 mg, 0.230 mmol) and compound 283 (340 mg, 0.783 mmol) and HATU (272 mg, 0.714 mmol) in DMF (5 mL) was added DIEA (220.4 mg, 1.705 mmol). The reaction mixture was stirred at room temperature for 10 min and then concentrated under reduced pressure. The resulting residue was purified by flash column chromatography to afford 289 (150 mg, 36% yield) as a white solid. ESI MS m/z: calcd for C₇₃H₁₀₃F₆N₂O₀₂₇ [M+H]⁺: 1805.71; found 1805.73.

### Example 188: rac-(2R,2′R)-2,2′-(((6R,9R)-1-((2S,3S,5S)-5-(4-amino-2-oxopyrimidin-1 (2H)-yl)-4,4-difluoro-3-hydroxytetrahydrofuran-2-yl)-16-(4-aminobutanamido)-6,9-dimethyl-5,8,11-trioxo-16-((3-oxopropoxy)methyl)-2,14,18-trioxa-4,7,10-triazahenicosan-21-oyl) bis(azanediyl))bis(N—((R)-1-(((((2S,3S,5S)-5-(4-amino-2-oxopyrimidin-1 (2H)-yl)-4,4-difluoro-3-hydroxytetrahydrofuran-2-yl)methoxy)methyl)amino)-1-oxopropan-2-yl) propanamide) (290)

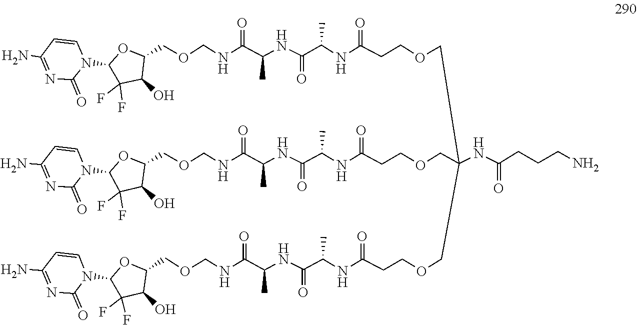 [SMILES: C[C@H](NC(=O)CCOCC(COCCC(=O)N[C@@H](C)C(=O)N[C@@H](C)C(=O)NCOC[C@H]1O[C@@H](n2ccc(N)nc2=O)C(F)(F)[C@@H]1O)(COCCC(=O)N[C@@H](C)C(=O)N[C@@H](C)C(=O)NCOC[C@H]1O[C@@H](n2ccc(N)nc2=O)C(F)(F)[C@@H]1O)NC(=O)CCCN)C(=O)N[C@@H](C)C(=O)NCOC[C@H]1O[C@@H](n2ccc(N)nc2=O)C(F)(F)[C@@H]1O]

To a solution of compound 289 (90 mg, 0.050 mmol) in MeOH (5 mL) at room temperature was added Pd/C (11 mg, 10 wt %). The resulting mixture was stirred at 50° C. under a H₂ balloon for 4 h before it was filtered through a pad of Celite. The filtrate was concentrated under reduced pressure to afford 290 (83 mg, 99% yield) as light yellow oil. ESI MS m/z: calcd for C₆₅H₉₇F₆N₂O₀₂₅ [M+H]⁺: 1671.68; found 1671.21.

### Example 189:2-((2,5,8,11,14,17,20,23-octaoxapentacosan-25-yl)carbamoyl)-4-((37S,45S,48S,51S)-45-((6S,9S)-1-((2R,3R,5R)-5-(4-amino-2-oxopyrimidin-1 (2H)-yl)-4,4-difluoro-3-hydroxytetrahydrofuran-2-yl)-16-((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-16-((6S,9S)-1-((2R,3R,5R)-5-(4-aminopyrimidin-1 (2H)-yl)-4-fluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18,23-pentaoxo-2,14-dioxa-4,7,10,17,22-pentaazapentacosan-25-yl)-37-(4-(2,5-dioxo-2,5-dihydro-1H-pyrrol-1-yl) butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10, 13,15-hexahydro-1H, 12H-benzo[de]pyrano[3′,4′: 6,7]indolizino[1,2-b]quinolin-1-yl)carbamate (292)

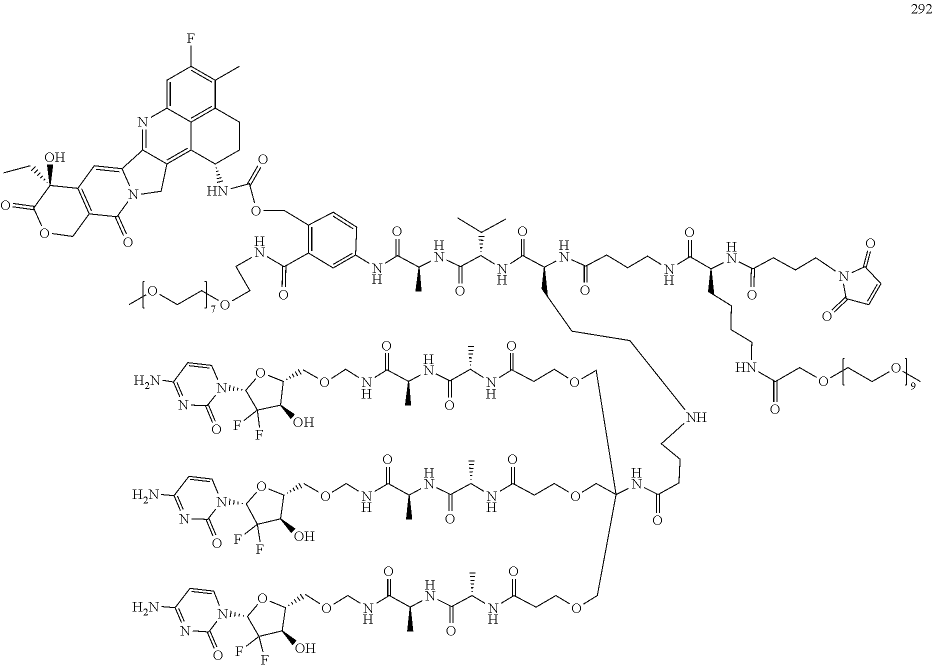

To a solution of compound 290 (106 mg, 0.050 mmol) in DMF (3 mL) was added HATU (20 mg, 0.055 mmol) at room temperature. The mixture was stirred at room temperature for 10 min. Then compound 291 (83 mg, 0.050 mmol) and DIEA (10 mg, 0.075 mmol) were added. The reaction mixture was stirred at room temperature for 0.5 h and then quenched by formic acid (0.5 mL), purified by prep-HPLC to afford 292 (51 mg, 27% yield) as a white solid. ESI MS m/z: calcd for C₁₆₇H₂₄₈F₆N32059 [M+2H]²⁺: 1880.87 found 1880.87.

### Example 190: benzyl (1-((2R,4R,5R)-4-(((benzyloxy)carbonyl)oxy)-5-(((di-tert-butoxyphosphaneyl)oxy)methyl)-3,3-difluorotetrahydrofuran-2-yl)-2-oxo-1,2-dihydropyrimidin-4-yl) carbamate (296)

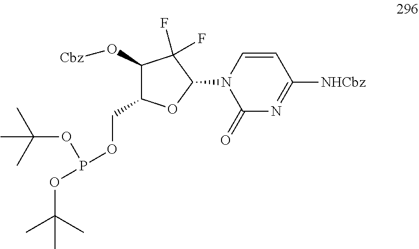 [SMILES: CC(C)(C)OP(OC[C@H]1O[C@@H](n2ccc(NC(=O)OCc3ccccc3)nc2=O)C(F)(F)[C@@H]1OC(=O)OCc1ccccc1)OC(C)(C)C]

To a solution of compound 294 (2.00 g, 3.763 mmol) in DCM (5 mL) were added 1H-tetrazole (0.53 g, 7.526 mmol) and compound 295 (2.09 g, 7.526 mmol). The mixture was stirred at RT for 1 h, and used for the next step without work-up. MS ESI (m/z): calcd for C₃₃H₄₀F₂N₃O₁₀P [M+H−56]⁺: 708.24; found 652.61.

### Example 191: benzyl (1-((2R,4R,5R)-4-(((benzyloxy)carbonyl)oxy)-5-(((di-tert-butoxyphosphoryl) oxy) methyl)-3,3-difluorotetrahydrofuran-2-yl)-2-oxo-1,2-dihydropyrimidin-4-yl) carbamate (297)

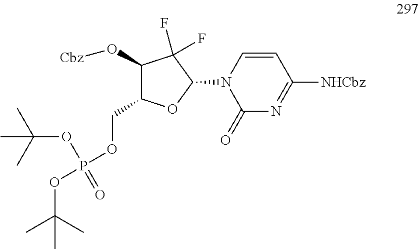 [SMILES: CC(C)(C)OP(=O)(OC[C@H]1O[C@@H](n2ccc(NC(=O)OCc3ccccc3)nc2=O)C(F)(F)[C@@H]1OC(=O)OCc1ccccc1)OC(C)(C)C]

To a solution of compound 296 (2.66 g, 3.763 mmol) was added m-CPBA (710 mg, 4.139 mmol) at −50° C. The mixture was stirred at −50° C. for 0.5 h and quenched with saturated Na₂SO₃ solution and extracted with DCM (30 mL×3). The organic layers were combined and dried over anhydrous Na₂SO₄ and concentrated in vacuo. The residue was purified by silica gel column chromatography (eluted with DCM/MeOH=20/1) to afford 297 (2.60 g, 95% yield) as a colorless oil. MS ESI (m/z): calcd for C₃₃H₄₀F₂N₃O₁₁P [M+H]⁺: 724.23; found 723.61.

### Example 192: ((2R,3R,5R)-5-(4-amino-2-oxopyrimidin-1 (2H)-yl)-4,4-difluoro-3-hydroxytetrahydrofuran-2-yl)methyl di-tert-butyl phosphate (298)

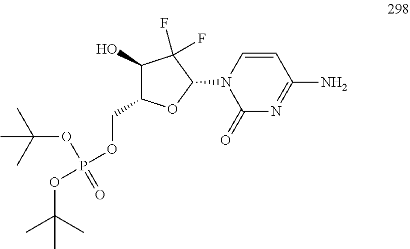 [SMILES: CC(C)(C)OP(=O)(OC[C@H]1O[C@@H](n2ccc(N)nc2=O)C(F)(F)[C@@H]1O)OC(C)(C)C]

To a solution of compound 297 (2.60 g, 3.674 mmol) in MeOH (20 mL) was added Pd/C (260 mg, 10 wt % Pd). The mixture was stirred at RT under H₂ for 2 h and filtered over Celite. The filtrate was concentrated to afford 298 (1.61 g, crude) as a white solid. MS ESI (m/z): calcd for C₁₇H₂₈F₂N₃O₇P [M+H]⁺: 456.16; found 455.81.

### Example 193: ((2R,3R,5R)-5-(4-amino-2-oxopyrimidin-1 (2H)-yl)-3-((tert-butyldimethylsilyl) oxy)-4,4-difluorotetrahydrofuran-2-yl)methyl di-tert-butyl phosphate (299)

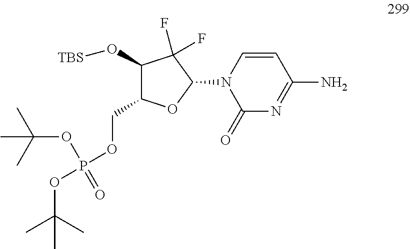

To a solution of compound 298 (1.61 g, 3.535 mmol) in DMF (5 mL) were added imidazole (0.96 g, 14.141 mmol) and TBSCl (1.07 g, 7.071 mmol). The residue was purified by silica gel column chromatography (eluted with DCM/MeOH=20/1˜15/1) to afford 299 (786 mg, 39% yield) as a white solid. MS ESI (m/z): calcd for C₂₃H₄₂F₂N₃O₇PSi [M+H]⁺: 570.25; found 569.71.

### Example 194: allyl((S)-1-(((R)-1-((3-((2,5,8,11,14,17,20,23-octaoxapentacosan-25-yl) carbamoyl)-4-(hydroxymethyl)phenyl)amino)-1-oxopropan-2-yl)amino)-3-methyl-1-oxobutan-2-yl)carbamate (301)

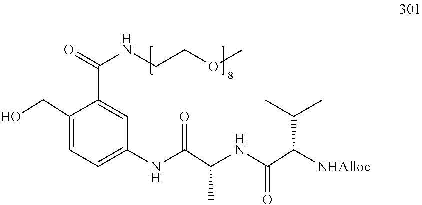

To a solution of compound 237 (1.90 g, 3.147 mmol) and compound 300 (0.63 g, 3.147 mmol) in DMF (10 mL) were added HATU (1.32 g, 3.46 mmol) and DIPEA (0.81 g, 6.294 mmol). The mixture was stirred at RT for 0.5 h, and then concentrated in vacuo. The residue was purified by column chromatography (eluted with DCM/MeOH=15/1) to afford 301 (813 mg, 33% yield) as a yellow solid. MS ESI (m/z): calcd for C₃₇H₆₂N₄O₁₄ [M+H]⁺: 787.43; found 786.81.

### Example 195: allyl((S)-1-(((R)-1-((3-((2,5,8,11,14,17,20,23-octaoxapentacosan-25-yl) carbamoyl)-4-((((4-nitrophenoxy)carbonyl)oxy)methyl)phenyl)amino)-1-oxopropan-2-yl)amino)-3-methyl-1-oxobutan-2-yl)carbamate (302)

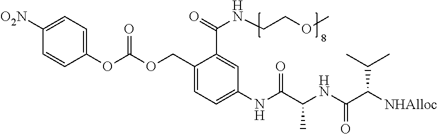

To a solution of compound 301 (1.42 g, 1.808 mmol) and 4-nitrophenyl carbonochloridate (729 mg, 3.617 mmol) in DCM (20 mL) was added pyridine (286 mg, 3.617 mmol). The mixture was stirred at RT for 0.5 h, and then concentrated in vacuo. The residue was purified by prep-HPLC (eluted with CH₃CN/H₂O=58/42) to afford 302 (1.26 g, 73% yield) as a yellow solid. MS ESI (m/z): calcd for C₄₄H₆₅N₅O₁₈ [M+H]⁺: 952.43; found 951.81.

### Example 196:2-((2,5,8,11,14,17,20,23-octaoxapentacosan-25-yl)carbamoyl)-4-((R)-2-((S)-2-(((allyloxy)carbonyl)amino)-3-methylbutanamido)propanamido)benzyl (1-((2R,4R,5R)-4-((tert-butyldimethylsilyl)oxy)-5-(((di-tert-butoxyphosphoryl)oxy)methyl)-3,3-difluorotetrahydrofuran-2-yl)-2-oxo-1,2-dihydropyrimidin-4-yl)carbamate (303)

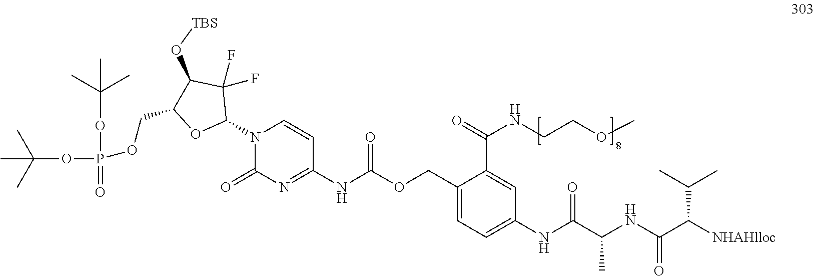

To a solution of compound 302 (300 mg, 0.527 mmol) in THF (5 mL) was added LiHMDS (1 mol/L in THF) (0.79 mL, 0.790 mmol) at −60° C. dropwise. The mixture was warmed to RT for 0.5 h and compound 299 (502 mg, 0.527 mmol) was added. After stirring at RT for 0.5 h, the reaction mixture was quenched with saturated NH₄Cl solution, then extracted with DCM (20 mL×3). The combined organic layers were dried over anhydrous Na₂SO₄ and concentrated in vacuo to afford 303 (728 mg, crude) as a yellow oil. MS ESI (m/z): calcd for C₆₁H₁₀₂F₂N₇O₂₂PSi [M+H]⁺: 1382.66; found 1381.94.

### Example 197:2-((2,5,8,11,14,17,20,23-octaoxapentacosan-25-yl)carbamoyl)-4-((R)-2-((S)-2-amino-3-methylbutanamido)propanamido)benzyl (1-((2R,4R,5R)-4-((tert-butyldimethylsilyl)oxy)-5-(((di-tert-butoxyphosphoryl)oxy)methyl)-3,3-difluorotetrahydrofuran-2-yl)-2-oxo-1,2-dihydropyrimidin-4-yl)carbamate (304)

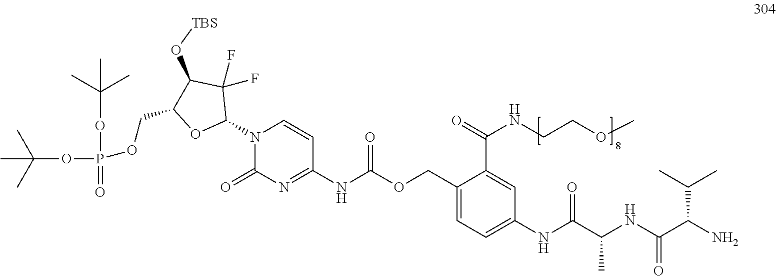

To a solution of compound 303 (728 mg, 0.527 mmol) in DCM (10 mL) were added Pd(PPh₃)₄ (30 mg, 0.026 mmol) and pyrrolidine (75 mg, 1.053 mmol). The mixture was stirred at RT for 0.5 h, and then concentrated in vacuo to afford 304 (683 mg, crude) as a yellow oil. MS ESI (m/z): calcd for C₅₇H₉₈F₂N₇O₂₀PSi [M+H]⁺: 1298.63; found 1298.91.

### Example 198:2-((2,5,8,11,14,17,20,23-octaoxapentacosan-25-yl) carbamoyl)-4-((R)-2-((S)-2-amino-3-methylbutanamido)propanamido)benzyl (1-((2R,4R,5R)-5-(((di-tert-butoxyphosphoryl)oxy)methyl)-3,3-difluoro-4-hydroxytetrahy drofuran-2-yl)-2-oxo-1,2-dihydropyrimidin-4-yl)carbamate (305)

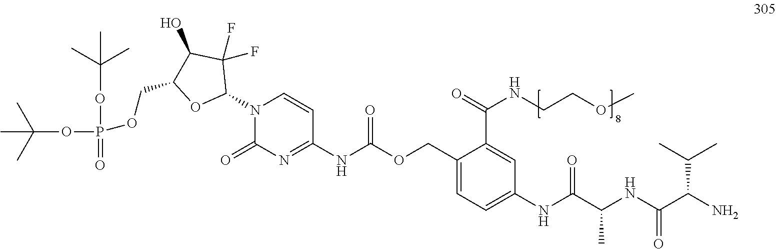

To a solution of compound 304 (683 mg, 0.526 mmol) in THF (10 mL) was added TBAF (1 mol/L in THF) (1.315 mL, 1.315 mmol). The mixture was stirred at RT for 0.5 h, and then concentrated in vacuo to afford 305 (300 mg, 48% yield in three steps) as a white solid. MS ESI (m/z): calcd for C₅₁H₈₄F₂N₇O₂₀P [M+H]⁺: 1184.55; found 1183.83.

### Example 199:2-((2,5,8,11,14,17,20,23-octaoxapentacosan-25-yl)carbamoyl)-4-((R)-2-((S)-2-amino-3-methylbutanamido)propanamido)benzyl (1-((2R,4R,5R)-3,3-difluoro-4-hydroxy-5-((phosphonooxy)methyl)tetrahydrofuran-2-yl)-2-oxo-1,2-dihydropyrimidin-4-yl)carbamate (306)

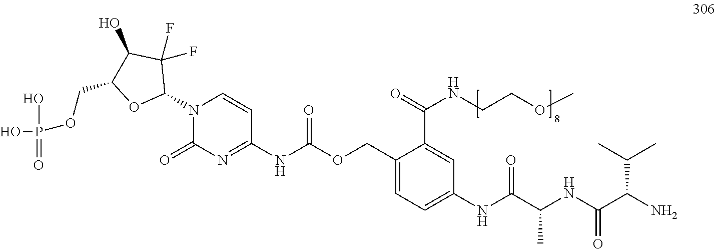

To a solution of compound 305 (35 mg, 0.030 mmol) was added HCl/dioxane (5 mL, 4 mol/L). The mixture was stirred at RT for 0.5 h, and then filtered. The collected cake was washed with DCM and dried to afford 306 (31 mg, crude) as a white solid. MS ESI (m/z): calcd for C₄₃H₆₈F₂N₇O₂₀P [M+H]⁺: 1072.42; found 1072.21.

### Example 200: ((2R,3R,5R)-5-(4-amino-2-oxopyrimidin-1 (2H)-yl)-3-((tert-butyldimethylsilyl) oxy)-4,4-difluorotetrahydrofuran-2-yl)methyl di-tert-butyl phosphate (307)

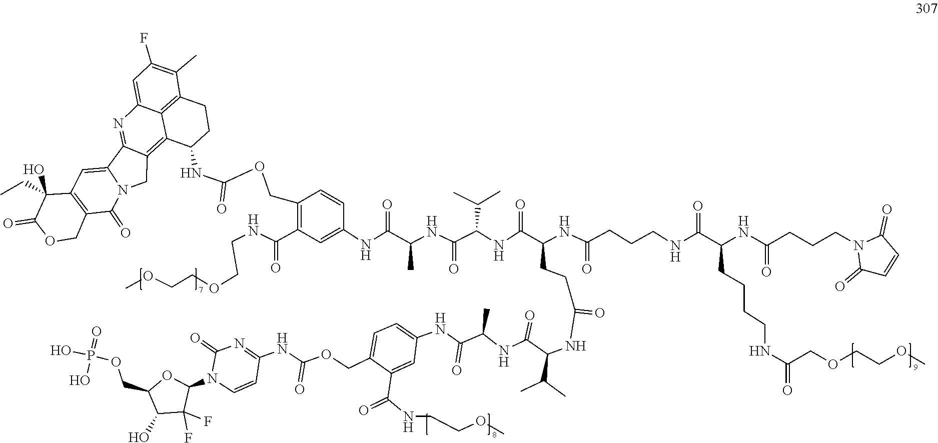

To a solution of compound 306 (32 mg, 0.030 mmol) and compound 291 (64 mg, 0.030 mmol) in DMF (2 mL) were added HATU (12 mg, 0.033 mmol) and DIPEA (15 mg, 0.119 mmol). The mixture was stirred at RT for 10 min and purified by prep-HPLC (eluted with CH₃CN/H₂O (containing 0.05% formic acid)=80/20) to afford 307 (7 mg, 7% yield) as a white solid. MS ESI (m/z): calcd for C₁₄₅H₂₁₇F₃N₁₉O₅₅P [M+2H]²⁺: 1597.23; found 1596.91.

### Example 201:2-((2,5,8,11,14,17,20,23-octaoxapentacosan-25-yl)carbamoyl)-4-((37S,45S,50S,53S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-50-isopropyl-53-methyl-31,38,43,48,51-pentaoxo-2,5,8,11,14,17,20,23,26,29-decaoxa-32,39,44,49,52-pentaazatetrapentacontan-54-amido)benzyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (311)

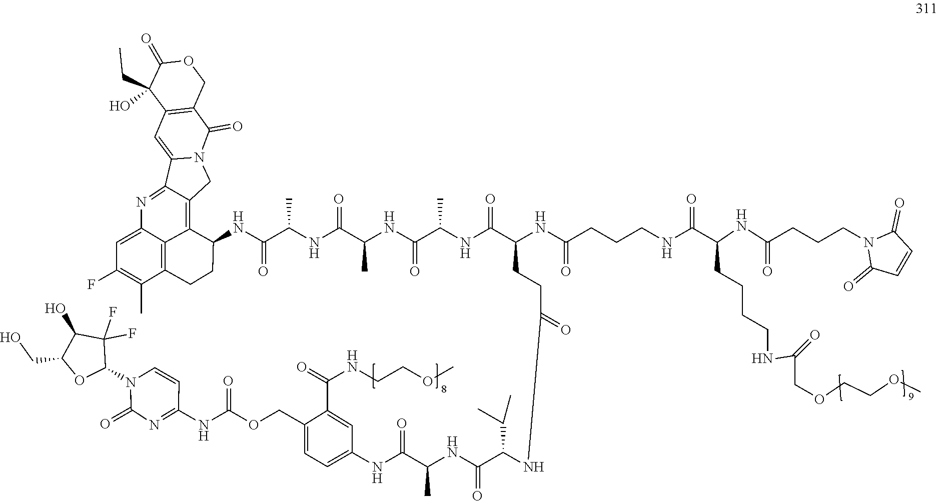

To a solution of compound 309 (70 mg, 0.071 mmol) and compound 310 (115 mg, 0.071 mmol) in DMF (2 mL) were added HATU (30 mg, 0.078 mmol) and DIPEA (28 mg, 0.212 mmol). The mixture was stirred at RT for 10 min and then purified by prep-HPLC (eluted with CH₃CN/H₂O (containing 0.05% formic acid)=43/57) to afford 311 (75 mg, 40% yield) as a white solid. MS ESI (m/z): calcd for C₁₂₀H₁₇₅F₃N₁₈O₄₂ [M+2H]²⁺: 1299.61; found 1298.71.

### Example 202: (2S)-N5-((6S,9S)-1-((2R,3R,5R)-5-(4-amino-2-oxopyrimidin-1 (2H)-yl)-4,4-difluoro-3-hydroxytetrahydrofuran-2-yl)-16-((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-16-((6S,9S)-1-((2R,3R,5R)-5-(4-aminopyrimidin-1 (2H)-yl)-4-fluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18-tetraoxo-2,14-dioxa-4,7,10,17-tetraazahenicosan-21-yl)-2-((S)-37-(4-(2,5-dioxo-2,5-dihydro-1H-pyrrol-1-yl) butanamido)-31,38-dioxo-2,5,8, 11, 14, 17,20,23,26,29-decaoxa-32,39-diazatritetracontan-43-amido)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl) pentanediamide (313)

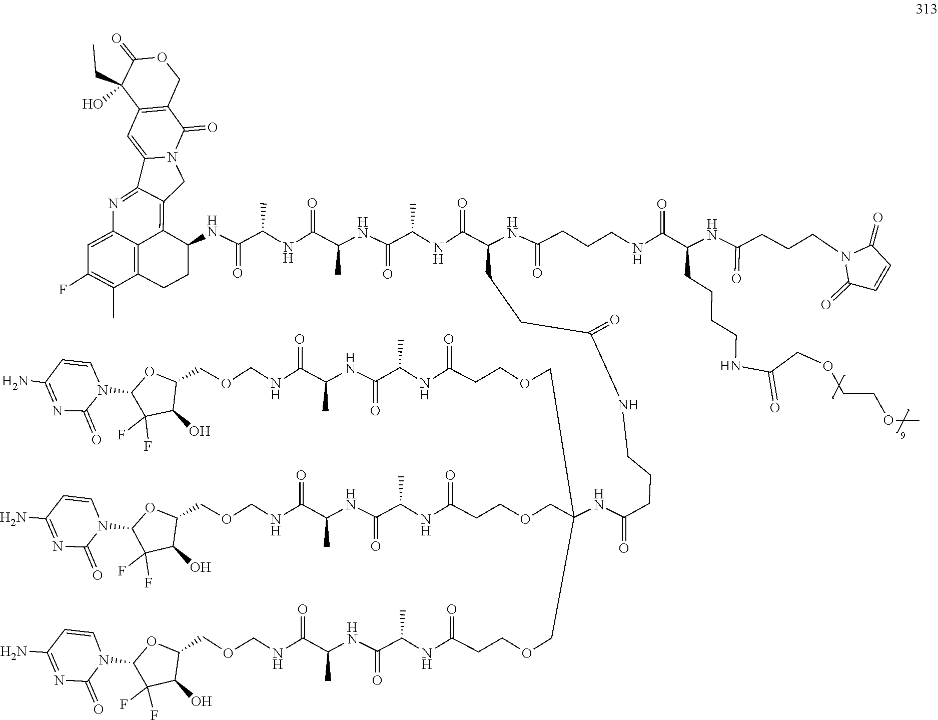

To a solution of compound 309 (243 mg, 0.150 mmol) in DMF (3 mL) was added HATU (63 mg, 0.165 mmol) at room temperature. The mixture was stirred at room temperature for 10 min. Then compound 290 (250.0 mg, 0.150 mmol) and DIEA (29 mg, 0.225 mmol) were added. The reaction mixture was stirred at room temperature for 0.5 h, and then quenched by formic acid (0.5 mL), purified by prep-HPLC to afford 313 (105 mg, 21% yield) as a white solid. ESI MS m/z: calcd for C₁₄₂H₂₀₅F₇N₃₁O₅₀ [M+2H]²⁺: 1639.22; found 1639.35.

### Example 203: (S)-5-(((S)-1-(((R)-1-((3-((2,5,8,11,14,17,20,23-octaoxapentacosan-25-yl) carbamoyl)-4-(((((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl) carbamoyl)oxy)methyl)phenyl)amino)-1-oxopropan-2-yl)amino)-3-methyl-1-oxobutan-2-yl)amino)-4-amino-5-oxopentanoic acid (316)

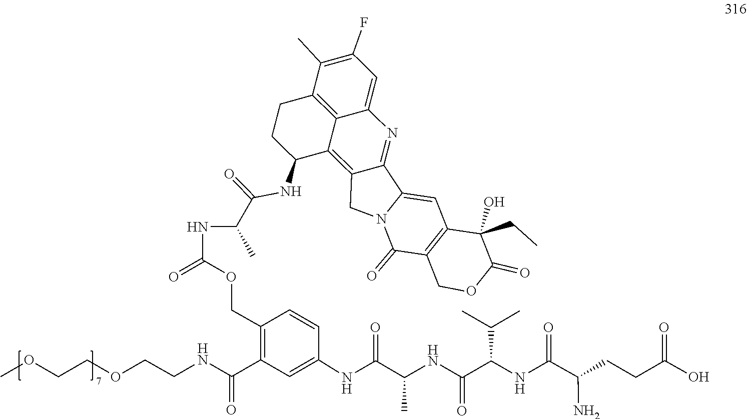

To a solution of compound 315 (220 mg, 0.139 mmol) in DMF (3.0 mL) was added piperidine (24 mg, 0.277 mmol). The reaction mixture was stirred at room temperature for 0.5 h, and then concentrated under reduced pressure to afford 316 (190 mg, 100% yield) as a yellow oil. ESI MS m/z: calcd for C₆₆H₉₁FN₉O₂₁ [M+H]⁺: 1364.62; found 1364.51.

### Example 204: perfluorophenyl(S)-37-(4-(2,5-dioxo-2,5-dihydro-1H-pyrrol-1-yl) butanamido)-31,38-dioxo-2,5,8,11,14,17,20,23,26,29-decaoxa-32,39-diazatritetracontan-43-oate (317)

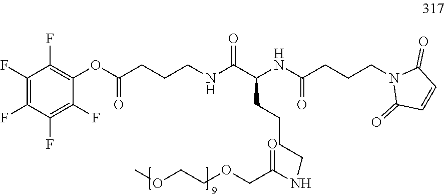

To a solution of compound 260 (2.00 g, 2.312 mmol) in DCM (20 mL) were added PFP—OH (0.55 g, 3.006 mmol) and EDCI (0.66 g, 3.468 mmol). The mixture was stirred at room temperature for 2 h, and then diluted with DCM (20 mL), washed with brine (5 mL×2). The solution was concentrated under reduced pressure to afford 317 (1.50 g, 63% yield) as a white solid. ESI MS m/z: calcd for C₄₅H₆₈F₅N₄O₁₇ [M+H]⁺: 1031.44; found 1031.02.

### Example 205: (37S,45S)-45-(((S)-1-(((R)-1-((3-((2,5,8,11,14,17,20,23-octaoxapentacosan-25-yl)carbamoyl)-4-(((((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl) butanamido)-31,38,43-trioxo-2,5,8,11,14,17,20,23,26,29-decaoxa-32,39,44-triazaoctatetracontan-48-oic acid (318)

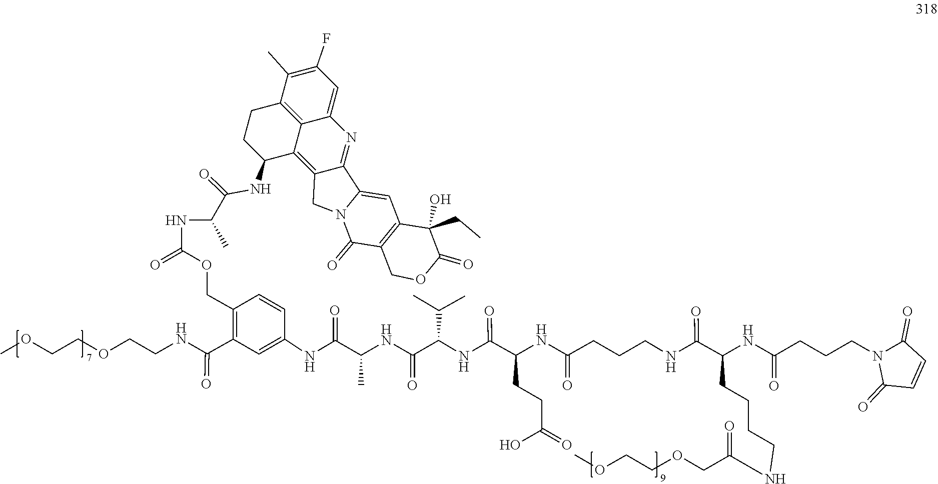

To a solution of compound 316 (190 mg, 0.139 mmol) in DMF (3 mL) were added compound 317 (287 mg, 0.278 mmol) and DIEA (45 mg, 0.348 mmol). The mixture was stirred at room temperature for 1 h and concentrated under reduced pressure. The resulting residue was purified by prep-HPLC to afford 318 (100 mg, 32% yield) as a white solid. ESI MS m/z: calcd for C₁₀₅H₁₅₇FN₁₃O₃₇ [M+H]⁺: 2211.07; found 2211.68.

### Example 206:2-((2,5,8,11,14,17,20,23-octaoxapentacosan-25-yl)carbamoyl)-4-((37S,45S,48S,51R)-45-((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18,23-pentaoxo-2,14-dioxa-4, 7,10,17,22-pentaazapentacosan-25-yl)-37-(4-(2,5-dioxo-2,5-dihydro-1H-pyrrol-1-yl) butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamate (319)

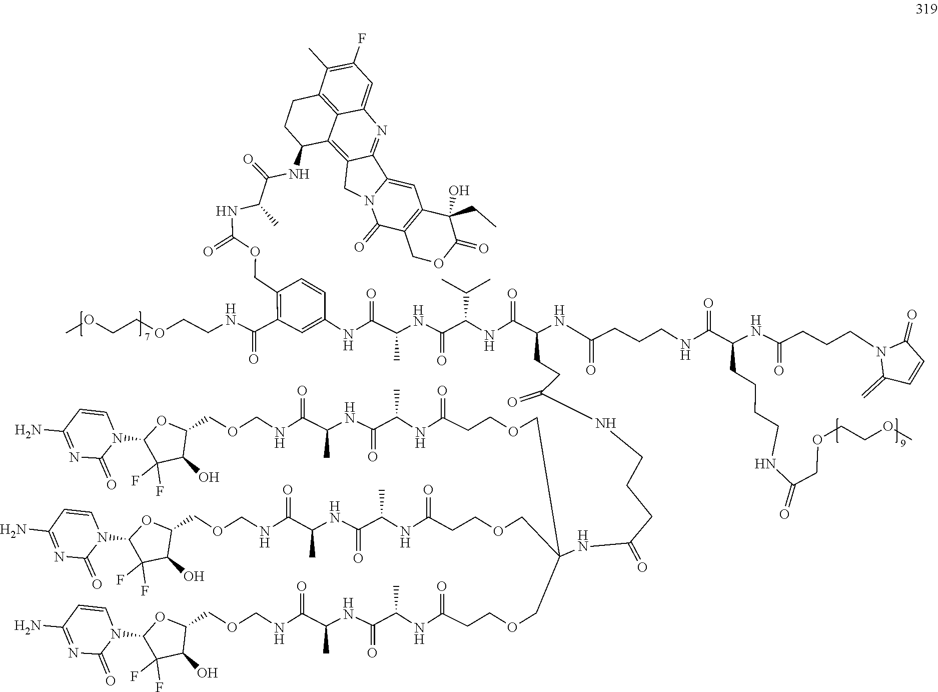

To a solution of compound 318 (100 mg, 0.045 mmol), compound 290 (76 mg, 0.045 mmol) and HATU (19 mg, 0.050 mmol) in DMF (4.0 mL) was added DIEA (9 mg, 0.068 mmol). The reaction mixture was stirred at room temperature for 0.5 h, and then quenched by formic acid (0.5 mL), purified by prep-HPLC to afford 319 (60 mg, 35% yield) as a white solid. ESI MS m/z: calcd for C₁₇₀H₂₅₁F₇N33061 [M+2H]²⁺: 1932.37 found 1932.77.

### Example 207: benzyl(S)-4-((((9H-fluoren-9-yl)methoxy)carbonyl)amino)-5-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12Hbenzo[de]pyrano[3′,4′: 6, 7]indolizino[1,2-b]quinolin-1-yl)amino)-1-oxopropan-2-yl)amino)-1-oxopropan-2-yl)amino)-1-oxopropan-2-yl)amino)-5-oxopentanoate (323)

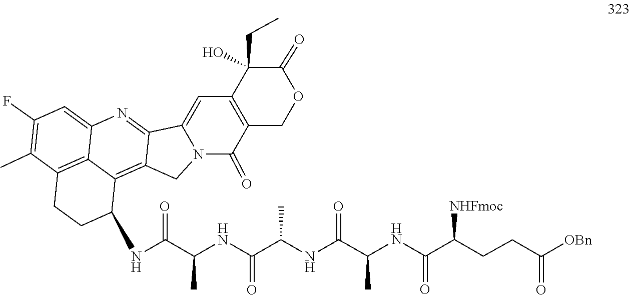

To a solution of compound 321 (300 mg, 0.462 mmol) and compound 245 (213 mg, 0.462 mmol) in DMF (4 mL) were added HATU (193 mg, 0.509 mmol) and DIPEA (179 mg, 1.387 mmol). The mixture was stirred at RT for 10 min and then concentrated in vacuo. The residue was purified by silica gel column chromatography (eluted with DCM/MeOH=20/1) to afford 323 (328 mg, 65% yield) as a white solid. MS ESI (m/z): calcd for C₆₀H₆₀FN₇O₁₂ [M+H]⁺: 1090.41; found 1089.72.

### Example 208: Synthesis of(S)-4-((((9H-fluoren-9-yl)methoxy)carbonyl)amino)-5-(((S)-1-(((S)-1-(((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6, 7]indolizino[1,2-b]quinolin-1-yl)amino)-1-oxopropan-2-yl)amino)-1-oxopropan-2-yl)amino)-1-oxopropan-2-yl)amino)-5-oxopentanoic acid (324)

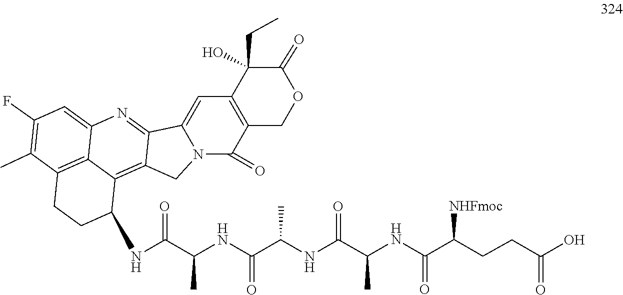

To a solution of compound 323 (150 mg, 0.138 mmol) in DMF (5 mL) was added Pd/C (15 mg, 10% Pd). The mixture was stirred at RT overnight under H₂ and then filtered over Celite. The filtrate was concentrated to afford 324 (137 mg, crude) as a white solid. MS ESI (m/z): calcd for C₅₃H₅₄FN₇O₁₂ [M+H]⁺: 999.41; found 999.62.

### Example 209: 2-((2,5,8,11,14,17,20,23-octaoxapentacosan-25-yl)carbamoyl)-4-((2S,5S,8S,11S,16S, 19S)-11-((((9H-fluoren-9-yl)methoxy)carbonyl)amino)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-16-isopropyl-2,5,8, 19-tetramethyl-1,4,7,10,14,17-hexaoxo-3,6,9,15,18-pentaazaicosan-20-amido)benzyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (325)

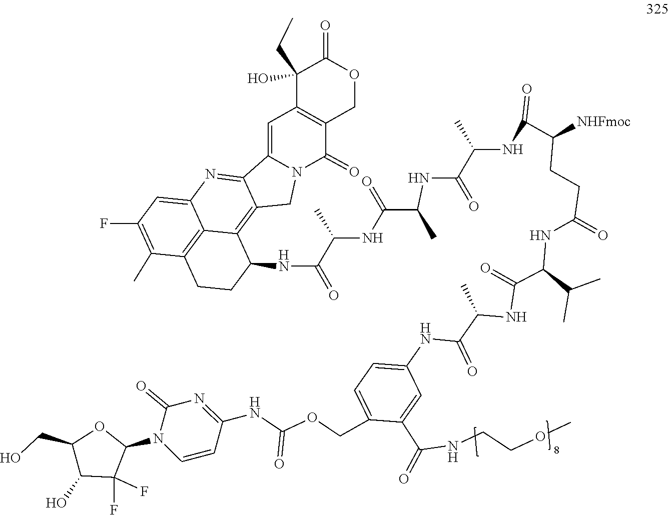

To a solution of compound 324 (100 mg, 0.101 mmol) and compound 310 (111 mg, 0.111 mmol) in DMF (4 mL) were added HATU (46 mg, 0.121 mmol) and DIPEA (26 mg, 0.202 mmol). The mixture was stirred at RT for 10 min and then concentrated in vacuo. The residue was purified by silica gel column chromatography (eluted with DCM/MeOH=7/1) to afford 325 (156 mg, 65% yield) as a yellow solid. MS ESI (m/z): calcd for C₉₆H₁₁₉F₃N₁₄O₂₈ [M+H]⁺: 1973.82; found 1973.81.

### Example 210: 2-((2,5,8,11, 14,17,20,23-octaoxapentacosan-25-yl)carbamoyl)-4-((2S,5S,8S,11S,16S, 19S)-11-amino-1-(((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6, 7]indolizino[1,2-b]quinolin-1-yl)amino)-16-isopropyl-2,5,8,19-tetramethyl-1,4,7,10,14,17-hexaoxo-3,6,9,15, 18-pentaazaicosan-20-amido)benzyl (1-((2R,4R,5R)-3,3-difluoro-4-hydroxy-5-(hydroxymethyl)tetrahydrofuran-2-yl)-2-oxo-1,2-dihvdropvrimidin-4-yl)carbamate (326)

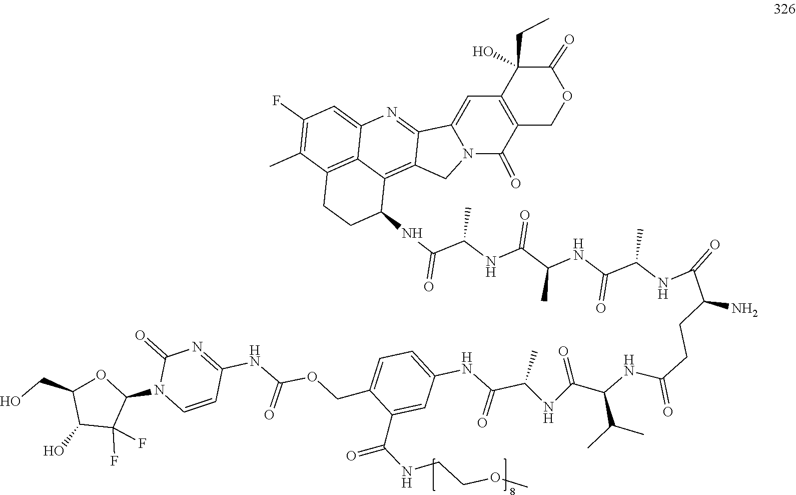

To a solution of compound 325 (156 mg, 0.079 mmol) in DMF (5 mL) was added piperidine (13 mg, 0.158 mmol). The mixture was stirred at RT for 1 h and then concentrated in vacuo. The residue was purified by silica gel column chromatography (DCM/MeOH=3/1) to afford 326 (92 mg, 66% yield) as a yellow solid. MS ESI (m/z): calcd for C₈₁H₁₀₉F₃N₁₄O₂₆ [M+H]⁺: 1751.81; found 1751.25.

### Example 211: 2-((2,5,8,11,14,17,20,23-octaoxapentacosan-25-yl)carbamoyl)-4-((2S,5S,8S,11S,28S,33S,36S)-11-(3-(((S)-1-(((S)-1-((4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)-3-((2-methoxyethyl)carbamoyl)phenyl)amino)-1-oxopropan-2-yl)amino)-3-methyl-1-oxobutan-2-yl)amino)-3-oxopropyl)-19,20-bis(2,5-dioxo-2,5-dihydro-1H-pyrrol-1-yl)-1-(((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6, 7]indolizino[1,2-b]quinolin-1-yl)amino)-28-(((S)-1-(((S)-1-(((S)-1-(((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6, 7]indolizino[1,2-b]quinolin-1-yl)amino)-1-oxopropan-2-yl)amino)-1-oxopropan-2-yl)amino)-1-oxopropan-2-yl)carbamoyl)-33-isopropyl-2,5,8,36-tetramethyl-1,4,7,10,13,18,21,26,31,34-decaoxo-3,6,9,12,17,22,27,32,35-nonaazaheptatriacontan-37-amido)benzyl (1-((2R,4R,5R)-3,3-difluoro-4-hydroxy-5-(hydroxymethyl)tetrahydrofuran-2-yl)-2-oxo-1,2-dihydropyrimidin-4-yl)carbamate (327)

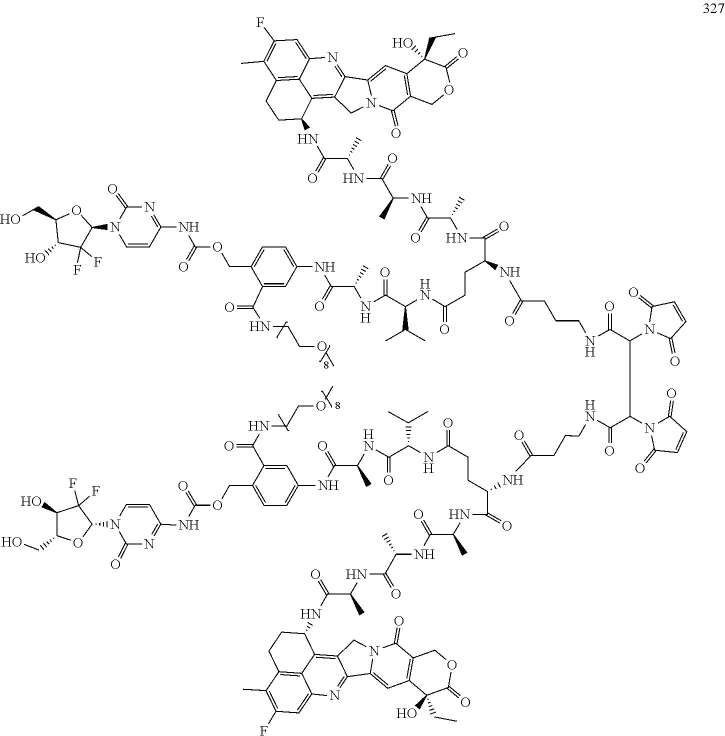

To a solution of compound 326 (64 mg, 0.036 mmol) and compound 257 (14 mg, 0.017 mmol) in DMF (2 mL) was added DIPEA (9 mg, 0.069 mmol). The mixture was stirred at RT for 1 h and purified by prep-HPLC (eluted with CH₃CN/H₂O (containing 0.05% formic acid)=40/60) to afford 327 (24 mg, 35% yield) as a white solid. MS ESI (m/z): calcd for C₁₈₂H₂₃₆F₆N₃₂O₆₀ [M+2H]²⁺: 1972.81; found 1972.90.

### Example 212: ((S)-4-((((2-((2,5,8,11,14,17,20,23-octaoxapentacosan-25-yl)carbamoyl)-4-((S)-2-((S)-2-(((allyloxy)carbonyl)amino)-3-methylbutanamido)propanamido)benzyl)oxy)carbonyl)oxy)-4-ethyl-8-fluoro-9-methoxy-3,14-dioxo-3,4,12, 14-tetrahydro-1H-pyrano[3′,4′: 6,7]indolizino[1,2-b]quinolin-11-yl)methyl acetate (331)

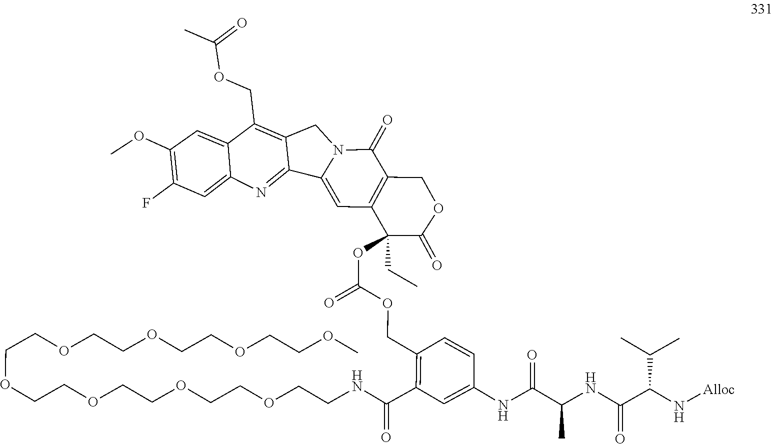

To a solution of compound 329 (600 mg, 1.280 mmol) in DCM (6 mL) were added triphosgene (228 mg, 0.760 mmol) and DMAP (782 mg, 6.400 mmol). After stirring at room temperature for 5 min, compound 330 (1.00 g, 1.280 mmol) in 6 mL DCM was added into the mixture. The reaction solution was stirred at room temperature for 20 min under N₂ atmosphere, and concentrated under reduced pressure. The resulting residue was purified by flash column chromatography to afford 331 (1.00 g, 65% yield) as a yellow solid. ESI MS m/z: calcd for C₆₂H₈₁FN₆O₂₂ [M+H]⁺: 1281.35; found 1281.58.

### Example 213: ((S)-4-((((2-((2,5,8,11,14,17,20,23-octaoxapentacosan-25-yl)carbamoyl)-4-((S)-2-((S)-2-amino-3-methylbutanamido)propanamido)benzyl)oxy)carbonyl)oxy)-4-ethyl-8-fluoro-9-methoxy-3,14-dioxo-3,4,12, 14-tetrahydro-1H-pyrano[3′,4′: 6, 7]indolizino[1,2-b]quinolin-11-yl)methyl acetate (332)

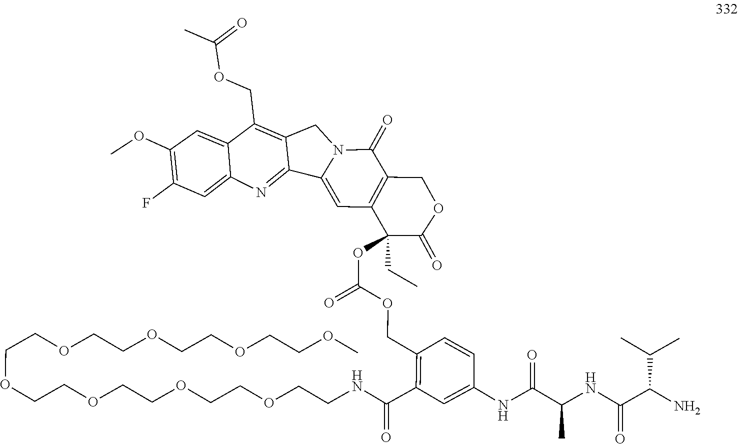 [SMILES: CC[C@@]1(OC(=O)OCc2ccc(NC(=O)[C@H](C)NC(=O)[C@@H](N)C(C)C)cc2C(=O)NCCOCCOCCOCCOCCOCCOCCOCCOC)C(=O)OCc2c1cc1n(c2=O)Cc2c-1nc1cc(F)c(OC)cc1c2COC(C)=O]

To a solution of compound 331 (1.00 g, 0.780 mmol) in DCM (6 mL) were added Pd(PPh₃)₄ (45 mg, 0.040 mmol) and tetrahydropyrrole (111 mg, 1.560 mmol). The reaction solution was stirred at room temperature for 1 h under N₂ atmosphere and concentrated under reduced pressure. The resulting residue was purified by flash column chromatography to afford 332 (830 mg, 88% yield) as a yellow oil product. ESI MS m/z: calcd for C₅₈H₇₇FN₆O₂₀ [M+H]⁺: 1198.27; found 1198.18.

### Example 214: allyl(S)-5-(((S)-1-(((S)-1-((3-((2,5,8,11,14,17,20,23-octaoxapentacosan-25-yl) carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6, 7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (334)

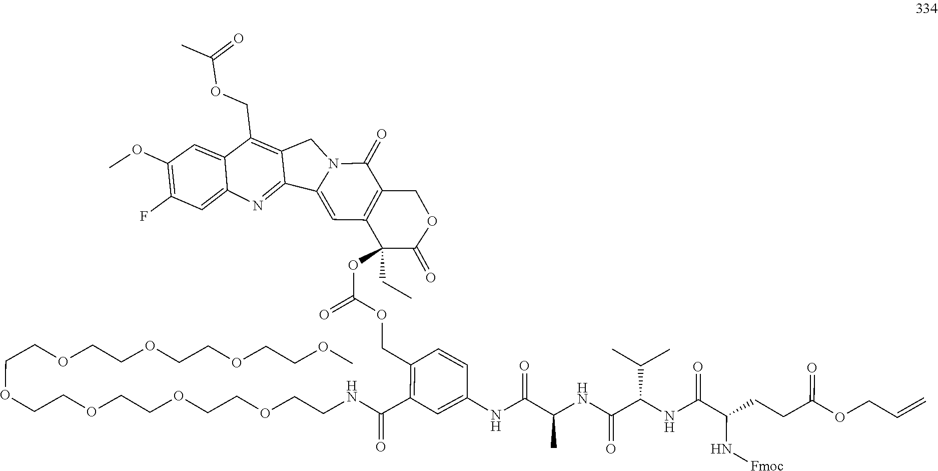

To a solution of compound 332 (830 mg, 0.690 mmol), compound 133 (312 mg, 0.760 mmol) and HATU (290 mg, 0.760 mmol) in DCM (10 mL) was added DIEA (134 mg, 1.030 mmol). The reaction solution was stirred at room temperature for 1 h and concentrated under reduced pressure. The resulting residue was purified by flash column chromatography to afford 334 (950 mg, 86% yield) as a yellow solid. ESI MS m/z: calcd for C₈₁H₉₈FN₇O₂₅ [M+H]⁺: 1588.66; found 1588.74.

### Example 215: (S)-5-(((S)-1-(((S)-1-((3-((2,5,8,11,14,17,20,23-octaoxapentacosan-25-yl) carbamoyl)-4-((((((S)-11-(acetoxymethyl)-4-ethyl-8-fluoro-9-methoxy-3,14-dioxo-3,4,12,14-tetrahydro-1H-pyrano[3′,4′: 6, 7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoic acid (335)

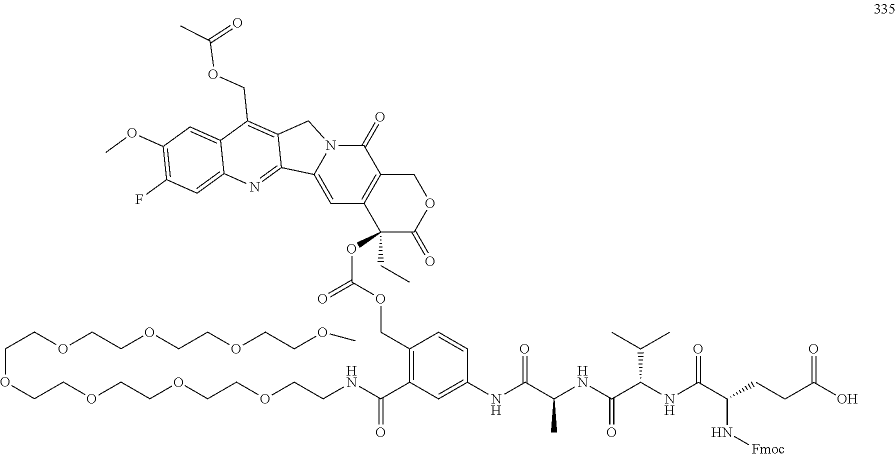

To a solution of compound 334 (200 mg, 0.125 mmol) in DCM (3 mL) were added Pd(PPh₃)₄ (7 mg, 0.006 mmol) and tetrahydropyrrole (18 mg, 0.250 mmol). The reaction solution was stirred at room temperature for 30 min under N₂ atmosphere and concentrated under reduced pressure. The resulting residue was purified by flash column chromatography to afford 335 (140 mg, 71% yield) as a yellow oil product. ESI MS m/z: calcd for C₇₈H₉₄FN₇O₂₅ [M+H]⁺: 1548.63; found 1547.73.

### Example 216: ((S)-4-((((2-((2,5,8,11,14,17,20,23-octaoxapentacosan-25-yl)carbamoyl)-4-((2S,5S,8S,13S,16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-((((9H-fluoren-9-yl)methoxy)carbonyl)amino)-5,13-diisopropyl-2,16-dimethyl-4,7,11,14,17-pentaoxo-3,6,12,15-tetraazaheptadecanamido)benzyl)oxy)carbonyl)oxy)-4-ethyl-8-fluoro-9-methoxy-3,14-dioxo-3,4,12,14-tetrahydro-1H-pyrano[3′,4′: 6,7]indolizino[1,2-b]quinolin-11-yl)methyl acetate (336)

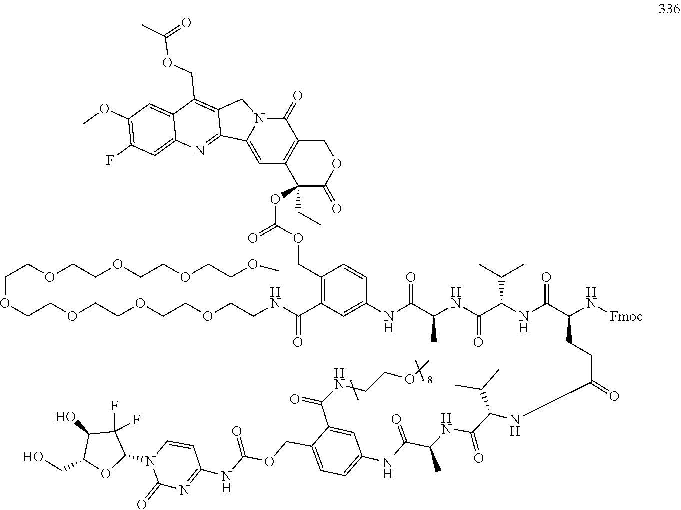

To a solution of compound 335 (140 mg, 0.090 mmol), compound 310 (90 mg, 0.090 mmol) and HATU (38 mg, 0.100 mmol) in DCM (2 mL) was added DIEA (18 mg, 0.140 mmol). The reaction solution was stirred at room temperature for 1 h and concentrated under reduced pressure. The resulting residue was purified by flash column chromatography to afford 336 (200 mg, 86% yield) as a yellow solid. ESI MS m/z: calcd for C₁₂₁H₁₅₉F₃N₁₄O₄₁ [M+2H]²⁺: 1261.54; found 1261.48.

### Example 217: ((S)-4-((((2-((2,5,8,11,14,17,20,23-octaoxapentacosan-25-yl)carbamoyl)-4-((2S,5S,8S,13S,16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-amino-5,13-diisopropyl-2,16-dimethyl-4,7,11,14,17-pentaoxo-3,6,12, 15-tetraazaheptadecanamido)benzyl)oxy)carbonyl)oxy)-4-ethyl-8-fluoro-9-methoxy-3,14-dioxo-3,4, 12,14-tetrahydro-1H-pyrano[3′,4′: 6, 7]indolizino[1,2-b]quinolin-11-yl)methyl acetate (337)

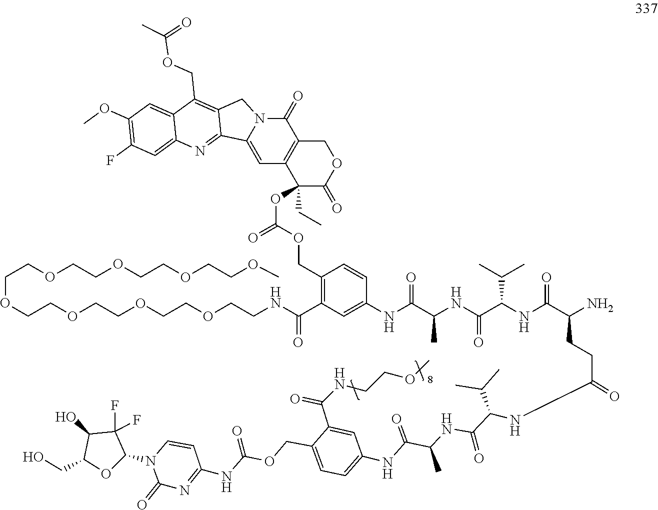

To a solution of compound 336 (200 mg, 0.080 mmol) in DMF (2.5 mL) was added piperidine (7 mg, 0.080 mmol). The reaction solution was stirred at room temperature for half an hour, and concentrated under reduced pressure. The resulting residue was purified by flash column chromatography to afford 337 (108 mg, 59% yield) as a yellow solid. ESI MS m/z: calcd for C₁₀₆H₁₄₉F₃N₁₄O₃₉ [M+2H]²⁺: 1150.52; found 1150.23.

### Example 218: ((S)-4-((((2-((2,5,8,11,14,17,20,23-octaoxapentacosan-25-yl)carbamoyl)-4-((2S,5S,8S,13S,16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-amino-5,13-diisopropyl-2, 16-dimethyl-4,7,11,14,17-pentaoxo-3,6,12, 15-tetraazaheptadecanamido)benzyl)oxy)carbonyl)oxy)-4-ethyl-8-fluoro-9-methoxy-3,14-dioxo-3,4,12, 14-tetrahydro-1H-pyrano[3′,4′: 6, 7]indolizino[1,2-b]quinolin-11-yl)methyl acetate (338)

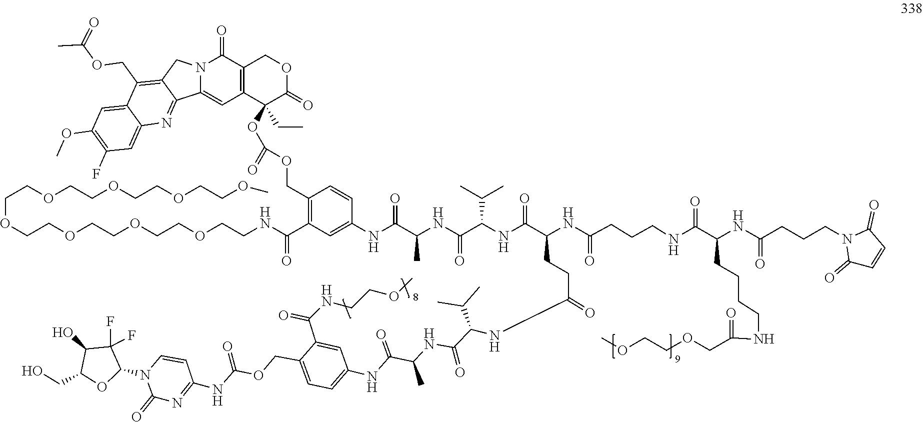

To a solution of compound 337 (108 mg, 0.040 mmol), compound 257 (37 mg, 0.040 mmol) and HATU (18 mg, 0.040 mmol) in DMF (2 mL) was added DIEA (8 mg, 0.060 mmol). The reaction solution was stirred at room temperature for 1 h, and concentrated under reduced pressure. The resulting residue was purified by prep-HPLC to afford 338 (52 mg, 36% yield) as light white solid. ESI MS m/z: calcd for C₁₃₈H₂₀₈F₃N₂₅O₄₈ [M+2H]²⁺: 1573.73; found 1574.18.

### Example 219: 2-((2,5,8,11,14, 17,20,23-octaoxapentacosan-25-yl)carbamoyl)-4-((S)-2-((S)-2-(((allyloxy)carbonyl)amino)-3-methylbutanamido)propanamido)benzyl((S)-1-(((1S,9S)-9-ethyl-5-fluoro-9-hydroxy-4-methyl-10, 13-dioxo-2,3,9,10,13, 15-hexahydro-1H, 12H-benzo[de]pyrano[3′,4′: 6,7] indolizino[1,2-b]quinolin-1-yl)amino)-1-oxopropan-2-yl)carbamate (342)

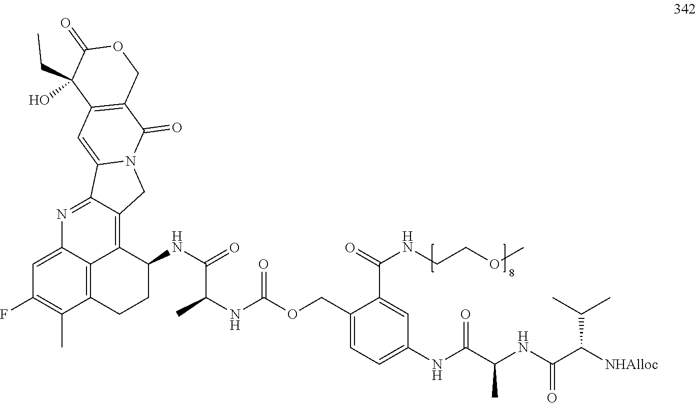

To a solution of compound 340 (478 mg, 0.502 mmol) and compound 341 (254 mg, 0.502 mmol) in DMF (5 mL) were added HOBt (68 mg, 0.502 mmol) and DIPEA (195 mg, 1.506 mmol). The mixture was stirred at RT for 2 h and concentrated in vacuo. The resulting residue was purified by silica gel column chromatography (eluted with DCM/MeOH=20/1) to afford 342 (660 mg, 99% yield) as a yellow solid. MS ESI (m/z): calcd for C₆₅H₈₇FN₈O₂₀ [M+H]⁺: 1319.61; found 1318.77.

### Example 220: 2-((2,5,8,11,14, 17,20,23-octaoxapentacosan-25-yl)carbamoyl)-4-((S)-2-((S)-2-amino-3-methylbutanamido)propanamido)benzyl((S)-1-(((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)amino)-1-oxopropan-2-yl)carbamate (343)

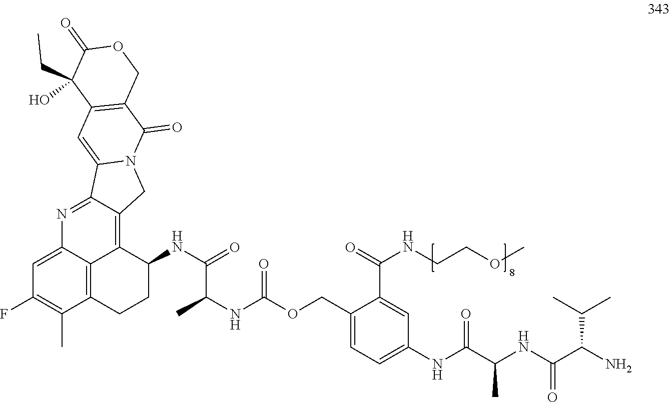

To a solution of compound 342 (660 mg, 0.500 mmol) in DCM (5 mL) were added Pd(PPh₃)₄ (29 mg, 0.025 mmol) and pyrrolidine (71 mg, 1.000 mmol). The mixture was stirred at RT for 0.5 h, and concentrated in vacuo. The residue was purified by silica gel column chromatography (eluted with DCM/MeOH=10/1) to afford 343 (617 mg, 99% yield) as a yellow solid. MS ESI (m/z): calcd for C₆₁H₈₃FN₈O₁₈ [M+H]⁺: 1235.59; found 1234.81.

### Example 221: benzyl(S)-5-(((S)-1-(((S)-1-((3-((2,5,8,11,14,17,20,23-octaoxapentacosan-25-yl) carbamoyl)-4-(((((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6, 7]indolizino[1,2-b]quinolin-1-yl)amino)-1-oxopropan-2-yl)carbamoyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (344)

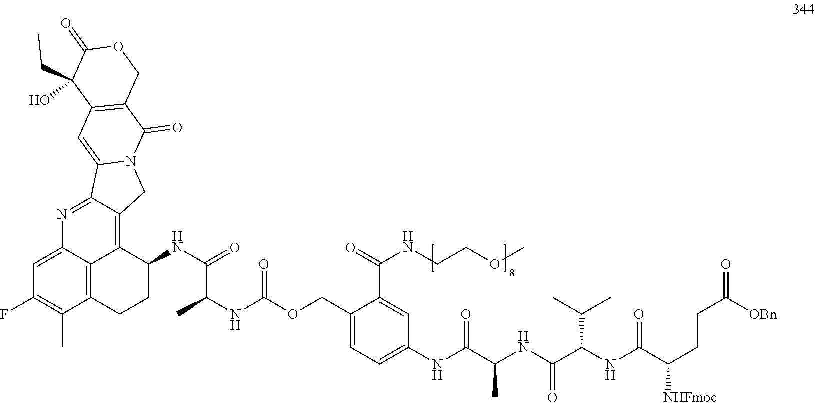

To a solution of compound 343 (617 mg, 0.499 mmol) and compound 245 (230 mg, 0.499 mmol) in DMF (5 mL) were added HATU (209 mg, 0.549 mmol) and DIPEA (129 mg, 0.999 mmol). The mixture was stirred at RT for 10 min and concentrated in vacuo. The residue was purified by silica gel column chromatography (eluted with DCM/MeOH=20/1) to afford 344 (588 mg, 65% yield) as a yellow solid. MS ESI (m/z): calcd for C₈₈H₁₀₆FN₉O₂₃ [M+H]⁺: 1676.75; found 1677.00.

### Example 222: (S)-5-(((S)-1-(((S)-1-((3-((2,5,8,11,14,17,20,23-octaoxapentacosan-25-yl) carbamoyl)-4-(((((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6, 7]indolizino[1,2-b]quinolin-1-yl)amino)-1-oxopropan-2-yl) carbamoyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoic acid (345)

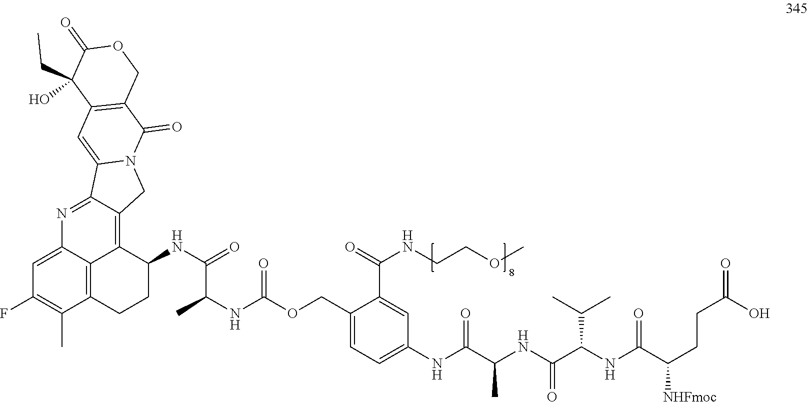

To a solution of compound 344 (588 mg, 0.351 mmol) in DMF (5 mL) was added Pd/C (60 mg, 10 wt %). The mixture was stirred at RT overnight under H₂ and filtered over Celite. The filtrate was concentrated in vacuo to afford 345 (556 mg, crude) as a yellow solid. MS ESI (m/z): calcd for C₈₁H₁₀₀FN₉O₂₃ [M+H]⁺: 1586.70; found 1585.46.

### Example 223: 2-((2,5,8,11,14,17,20,23-octaoxapentacosan-25-yl)carbamoyl)-4-((2S,5S,8S,13S, 16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-((((9H-fluoren-9-yl)methoxy)carbonyl)amino)-5,13-diisopropyl-2,16-dimethyl-4,7,11,14,17-pentaoxo-3,6,12,15-tetraazaheptadecanamido)benzyl ((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamate (346)

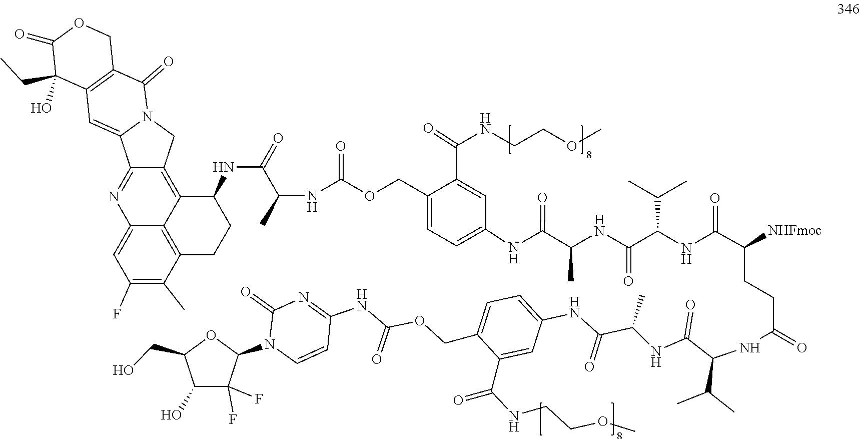

To a solution of compound 345 (160 mg, 0.101 mmol) and compound 310 (100 mg, 0.101 mmol) in DMF (5 mL) were added HATU (46 mg, 0.121 mmol) and DIPEA (26 mg, 0.202 mmol). The mixture was stirred at RT for 10 min and concentrated in vacuo. The residue was purified by silica gel column chromatography (eluted with DCM/MeOH=7/1) to afford 346 (181 mg, 65% yield) as a yellow solid. MS ESI (m/z): calcd for C₁₂₄H₁₆₅F₃N₁₆O₃₉ [M+2H]²⁺: 1280.57; found 1280.27.

### Example 224: 2-((2,5,8,11,14,17,20,23-octaoxapentacosan-25-yl)carbamoyl)-4-((2S,5S,8S,13S, 16S)-17-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-8-amino-5,13-diisopropyl-2,16-dimethyl-4,7,11,14,17-pentaoxo-3,6, 12,15-tetraazaheptadecanamido)benzyl ((S)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1-oxopropan-2-yl)carbamate (347)

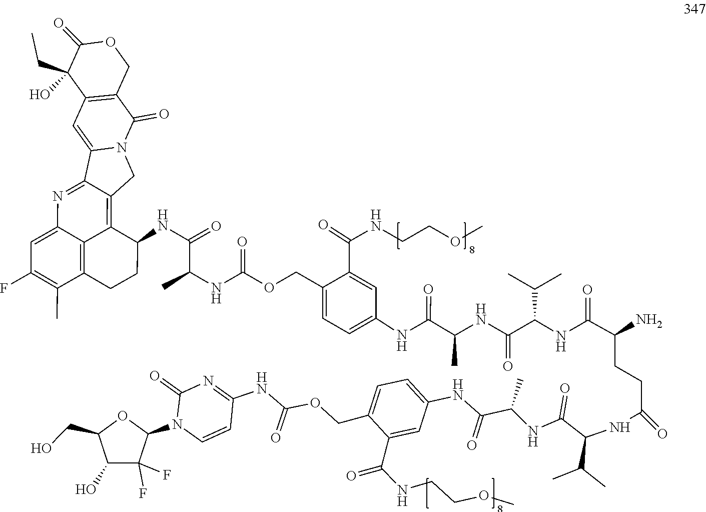

To a solution of compound 346 (181 mg, 0.071 mmol) in DMF (5 mL) was added piperidine (9 mg, 0.106 mmol). The mixture was stirred at RT for 1 h and concentrated in vacuo to afford 347 (165 mg, crude) as a yellow solid. MS ESI (m/z): calcd for C₁₀₉H₁₅₅F₃N₁₆O₃₇ [M+H]⁺: 2338.08; found 2337.84.

### Example 225: 2-((2,5,8,11,14,17,20,23-octaoxapentacosan-25-yl)carbamoyl)-4-((37S,45S,48S,51S)-45-(3-(((S)-1-(((S)-1-((3-((2,5,8,11,14,17,20,23-octaoxapentacosan-25-yl)carbamoyl)-4-((((1-((2R,4R,5R)-3,3-difluoro-4-hydroxy-5-(hydroxymethyl)tetrahydrofuran-2-yl)-2-oxo-1,2-dihydropyrimidin-4-yl)carbamoyl)oxy)methyl)phenyl)amino)-1-oxopropan-2-yl)amino)-3-methyl-1-oxobutan-2-yl)amino)-3-oxopropyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamate (348)

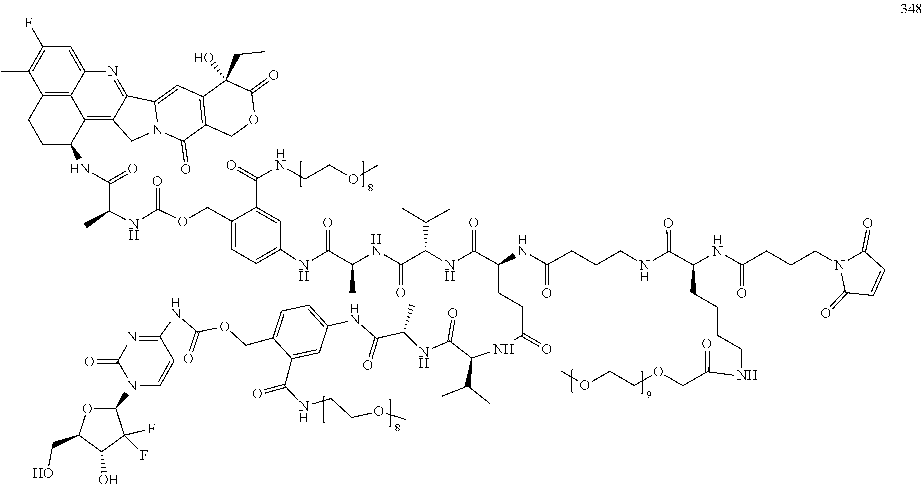

To a solution of compound 347 (165 mg, 0.071 mmol) and compound 57 (61 mg, 0.071 mmol) in DMF (5 mL) were added HATU (30 mg, 0.078 mmol) and DIPEA (18 mg, 0.141 mmol). The mixture was stirred at RT for 10 min and purified by prep-HPLC (eluted with CH₃CN/H₂O (0.05% FA)=40/60) to afford 348 (90 mg, 40% yield) as a white solid. MS ESI (m/z): calcd for C₁₄₈H₂₂₁F₃N₂₀O₅₃ [M+2H]²⁺ 1592.76; found 1592.33.

### Example 226: ((S)-4-((((2-((2,5,8,11,14,17,20,23-octaoxapentacosan-25-yl)carbamoyl)-4-((6S,9S,26S,29S,32R)-26-((((9H-fluoren-9-yl)methoxy)carbonyl)amino)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-29-isopropyl-6,9,32-trimethyl-5,8, 11,18,23,27,30-heptaoxo-2,14-dioxa-4,7,10,17,22,28,31-heptaazatritriacontan-33-amido)benzyl)oxy)carbonyl)oxy)-4-ethyl-8-fluoro-9-methoxy-3,14-dioxo-3,4,12,14-tetrahydro-1H-pyrano[3′,4′: 6,7]indolizino[1,2-b]quinolin-11-yl)methyl acetate (350)

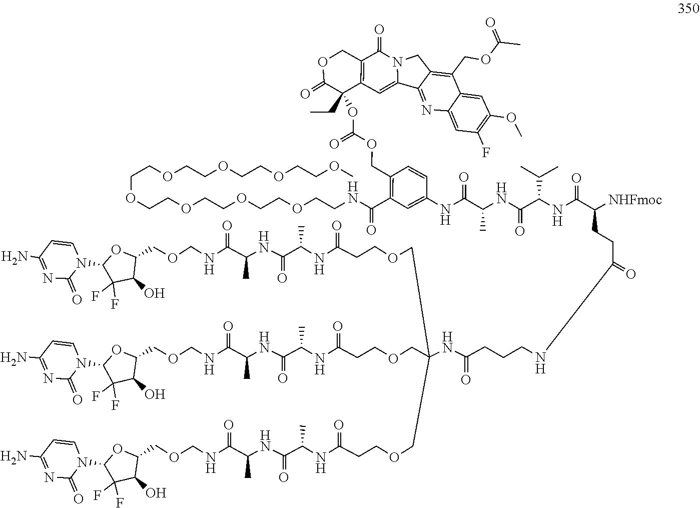

To a solution of compound 335 (100 mg, 0.065 mmol) and compound 290 (108 mg, 0.065 mmol) in DMF (5 mL) were added HATU (27 mg, 0.071 mmol) and DIPEA (17 mg, 0.129 mmol). The mixture was stirred at RT for 10 min and concentrated in vacuo. The residue was purified by prep-HPLC (eluted with CH₃CN/H₂O=45/55) to afford 350 (101 mg, 49% yield) as a white solid. MS ESI (m/z): calcd for C₁₄₃H₁₈₈F₇N₂₇O₄₉ [M+2H]₂₊: 1601.15; found 1601.72.

### Example 227: ((S)-4-((((2-((2,5,8,11,14,17,20,23-octaoxapentacosan-25-yl)carbamoyl)-4-((6S,9S,26S,29S,32R)-26-amino-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-29-isopropyl-6,9,32-trimethyl-5,8,11,18,23,27,30-heptaoxo-2,14-dioxa-4,7,10,17,22,28,31-heptaazatritriacontan-33-amido)benzyl)oxy)carbonyl)oxy)-4-ethyl-8-fluoro-9-methoxy-3,14-dioxo-3,4, 12,14-tetrahydro-1H-pyrano[3′,4′: 6,7]indolizino[1,2-b]quinolin-11-yl)methyl acetate (351)

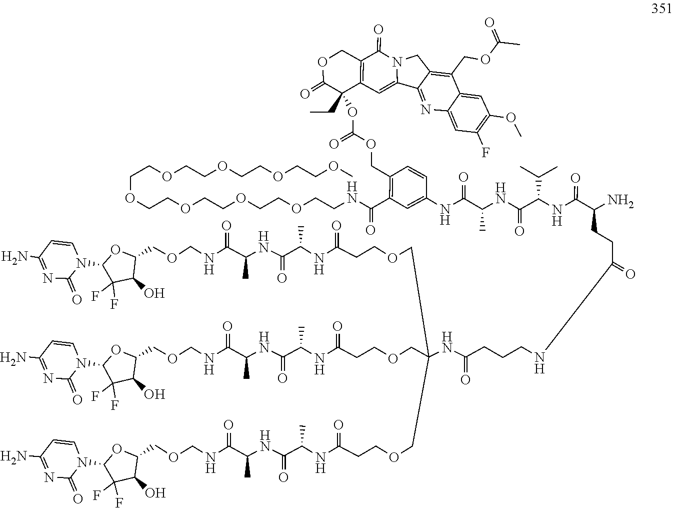 [SMILES: CC[C@@]1(OC(=O)OCc2ccc(NC(=O)[C@@H](C)NC(=O)[C@@H](NC(=O)[C@@H](N)CCC(=O)NCCCC(=O)NC(COCCC(=O)N[C@@H](C)C(=O)N[C@@H](C)C(=O)NCOC[C@H]3O[C@@H](n4ccc(N)nc4=O)C(F)(F)[C@@H]3O)(COCCC(=O)N[C@@H](C)C(=O)N[C@@H](C)C(=O)NCOC[C@H]3O[C@@H](n4ccc(N)nc4=O)C(F)(F)[C@@H]3O)COCCC(=O)N[C@@H](C)C(=O)N[C@@H](C)C(=O)NCOC[C@H]3O[C@@H](n4ccc(N)nc4=O)C(F)(F)[C@@H]3O)C(C)C)cc2C(=O)NCCOCCOCCOCCOCCOCCOCCOCCOC)C(=O)OCc2c1cc1n(c2=O)Cc2c-1nc1cc(F)c(OC)cc1c2COC(C)=O]

To a solution of compound 350 (101 mg, 0.032 mmol) in DMF (4 mL) was added piperidine (5 mg, 0.063 mmol). The mixture was stirred at RT for 1 h and then concentrated in vacuo to afford 351 (94 mg, crude) as a yellow solid. MS ESI (m/z): calcd for C₁₂₈H₁₇₈F₇N₂₇O₄₇ [M+2H]₂₊: 1490.12; found 1489.82.

### Example 228: Synthesis of ((S)-4-((((2-((2,5,8,11,14,17,20,23-octaoxapentacosan-25-yl)carbamoyl)-4-((37S,45S,48S,51R)-45-((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18,23-pentaoxo-2,14-dioxa-4,7,10,17,22-pentaazapentacosan-25-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl)oxy)carbonyl)oxy)-4-ethyl-8-fluoro-9-methoxy-3,14-dioxo-3,4,12,14-tetrahydro-1H-pyrano[3′,4′: 6,7]indolizino[1,2-b]quinolin-11-yl)methyl acetate (352)

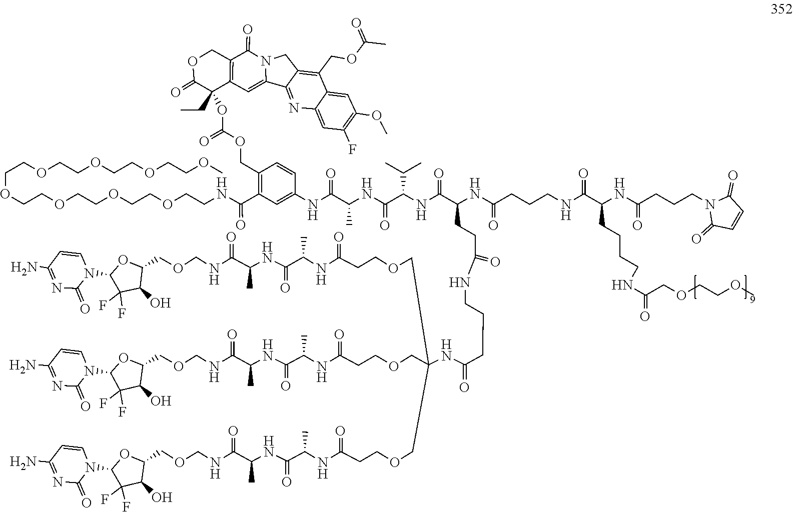

To a solution of compound 351 (94 mg, 0.032 mmol) and compound 260 (41 mg, 0.047 mmol) in DMF (5 mL) were added HATU (18 mg, 0.048 mmol) and DIPEA (8 mg, 0.063 mmol). The mixture was stirred at RT for 10 min and then purified by prep-HPLC (eluted with CH₃CN/H₂O (containing 0.05% formic acid)=38/62) to afford 352 (57 mg, 47% yield) as a white solid. MS ESI (m/z): calcd for C₁₆₇H₂₄₄F₇N₃₁O₆₃ [M+2H]₂₊: 1913.34; found 1913.65.

### Example 229: allyl(S)-5-(((S)-1-(((R)-1-((3-((2,5,8,11,14,17,20,23-octaoxapentacosan-25-yl) carbamoyl)-4-((((((S)-4-ethyl-8-fluoro-9-methoxy-11-((4-(methylsulfonyl)piperazin-1-yl)methyl)-3,14-dioxo-3,4, 12,14-tetrahydro-1H-pyrano[3′,4′: 6,7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (355)

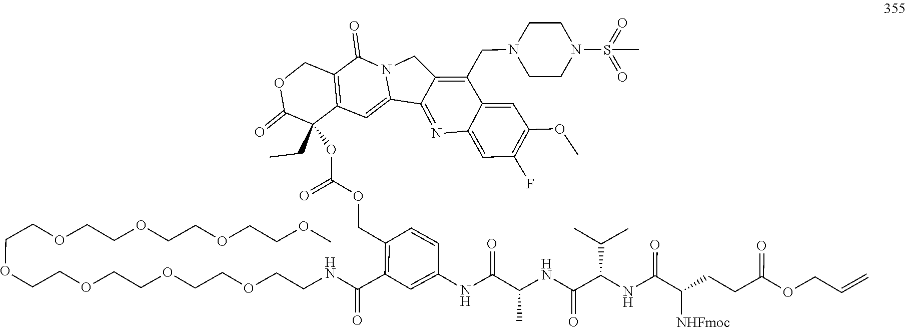

To a solution of compound 354 (187 mg, 0.144 mmol) and compound 133 (59 mg, 0.144 mmol) in DMF (5 mL) were added HATU (60 mg, 0.158 mmol) and DIPEA (37 mg, 0.287 mmol). The mixture was stirred at RT for 10 min and concentrated in vacuo. The residue was purified by silica gel column chromatography (eluted with DCM/MeOH=20/1) to afford 355 (183 mg, 75% yield) as a yellow solid. MS ESI (m/z): calcd for C₈₄H₁₀₆FN₉O₂₅S [M+H]⁺: 1692.71; found 1692.83.

### Example 230: Synthesis of(S)-5-(((S)-1-(((R)-1-((3-((2,5,8,11,14,17,20,23-octaoxapentacosan-25-yl)carbamoyl)-4-((((((S)-4-ethyl-8-fluoro-9-methoxy-11-((4-(methylsulfonyl)piperazin-1-yl)methyl)-3,14-dioxo-3,4, 12,14-tetrahydro-1H-pyrano[3′,4′: 6,7]indolizino[1,2-b]quinolin-4-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoic acid (356)

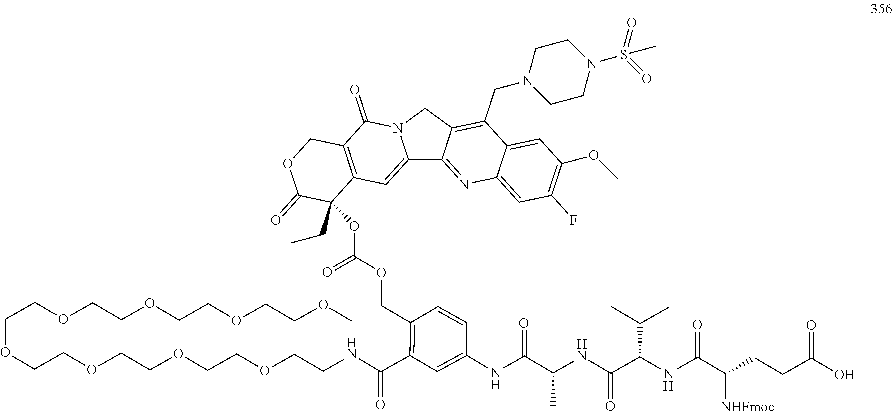

To a solution of compound 355 (187 mg, 0.110 mmol) in DCM (5 mL) was added Pd (PPh₃)₄ (6 mg, 0.006 mmol), then pyrrolidine (16 mg, 0.221 mmol). The mixture was stirred at RT for 15 min, and concentrated in vacuo. The residue was purified by silica gel column chromatography (eluted with DCM/MeOH=15/1) to afford 356 (100 mg, 55% yield) as a yellow solid. MS ESI (m/z): calcd for C₈₁H₁₀₂FN₉O₂₅S [M+H]⁺: 1652.68; found 1652.83.

### Example 231: (9H-fluoren-9-yl)methyl ((6S,9S,26S,29S,32R)-33-((3-((2,5,8,11,14,17,20,23-octaoxapentacosan-25-yl)carbamoyl)-4-((((((S)-4-ethyl-8-fluoro-9-methoxy-11-((4-(methylsulfonyl)piperazin-1-yl)methyl)-3,14-dioxo-3,4,12,14-tetrahydro-1H-pyrano[3′,4′: 6,7]indolizino[1,2-b]quinolin-4-yl)oxy)carbonyl)oxy)methyl)phenyl)amino)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-29-isopropyl-6,9,32-trimethyl-5,8,11,18,23,27,30,33-octaoxo-2,14-dioxa-4, 7, 10,17,22,28,31-heptaazatritriacontan-26-yl)carbamate (357)

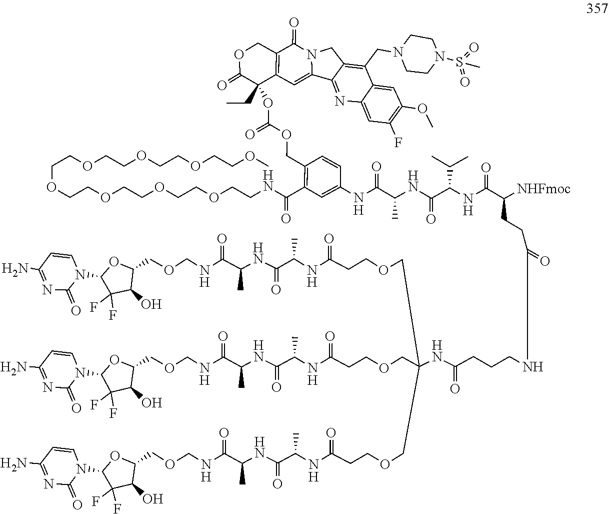

To a solution of compound 356 (100 mg, 0.061 mmol) and compound 290 (101 mg, 0.061 mmol) in DMF (5 mL) were added HATU (25 mg, 0.067 mmol) and DIPEA (16 mg, 0.121 mmol). The mixture was stirred at RT for 10 min and concentrated in vacuo. The residue was purified by prep-HPLC (eluted with CH₃CN/H₂O=50/50) to afford 357 (200 mg, 99% yield) as a white solid. MS ESI (m/z): calcd for C₁₄₆H₁₉₆F₇N₂₉O₄₉S [M+2H]₂₊: 1653.17; found 1654.22.

### Example 232: 2-((2,5,8,11,14,17,20,23-octaoxapentacosan-25-yl)carbamoyl)-4-((6S,9S,26S,29S,32R)-26-amino-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-29-isopropyl-6,9,32-trimethyl-5,8,11,18,23,27,30-heptaoxo-2,14-dioxa-4,7,10,17,22,28,31-heptaazatritriacontan-33-amido)benzyl ((S)-4-ethyl-8-fluoro-9-methoxy-11-((4-(methylsulfonyl)piperazin-1-yl)methyl)-3,14-dioxo-3,4,12,14-tetrahydro-1H-pyrano[3′,4′: 6,7]indolizino[1,2-b]quinolin-4-yl)carbonate (358)

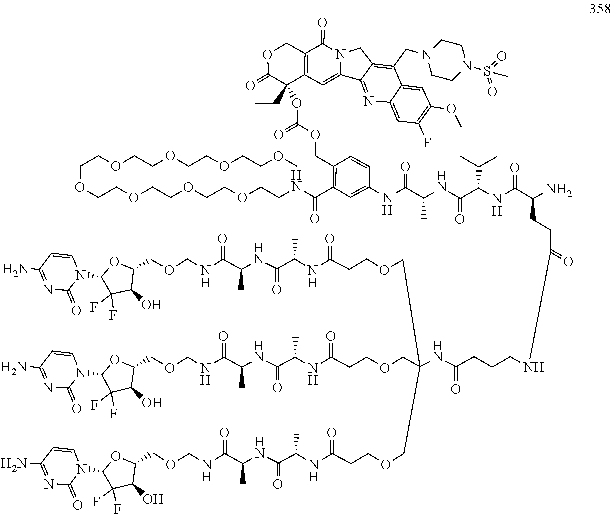 [SMILES: CC[C@@]1(OC(=O)OCc2ccc(NC(=O)[C@@H](C)NC(=O)[C@@H](NC(=O)[C@@H](N)CCC(=O)NCCCC(=O)NC(COCCC(=O)N[C@@H](C)C(=O)N[C@@H](C)C(=O)NCOC[C@H]3O[C@@H](n4ccc(N)nc4=O)C(F)(F)[C@@H]3O)(COCCC(=O)N[C@@H](C)C(=O)N[C@@H](C)C(=O)NCOC[C@H]3O[C@@H](n4ccc(N)nc4=O)C(F)(F)[C@@H]3O)COCCC(=O)N[C@@H](C)C(=O)N[C@@H](C)C(=O)NCOC[C@H]3O[C@@H](n4ccc(N)nc4=O)C(F)(F)[C@@H]3O)C(C)C)cc2C(=O)NCCOCCOCCOCCOCCOCCOCCOCCOC)C(=O)OCc2c1cc1n(c2=O)Cc2c-1nc1cc(F)c(OC)cc1c2CN1CCN(S(C)(=O)=O)CC1]

To a solution of compound 357 (200 mg, 0.060 mmol) in DMF (4 mL) was added piperidine (10 mg, 0.121 mmol). The mixture was stirred at RT for 1 h and then concentrated in vacuo to afford 358 (186 mg, crude) as a yellow solid. MS ESI (m/z): calcd for C₁₃₁H₁₈₆F₇N₂₉O₄₇S [M+2H]₂₊: 1542.14; found 1542.13.

### Example 233: 2-((2,5,8,11, 14, 17,20,23-octaoxapentacosan-25-yl)carbamoyl)-4-((37S,45S,48S,51R)-45-((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18,23-pentaoxo-2,14-dioxa-4,7,10,17,22-pentaazapentacosan-25-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((S)-4-ethyl-8-fluoro-9-methoxy-11-((4-(methylsulfonyl)piperazin-1-yl)methyl)-3,14-dioxo-3,4, 12,14-tetrahydro-1H-pyrano[3′,4′: 6,7]indolizino[1,2-b]quinolin-4-yl)carbonate (359)

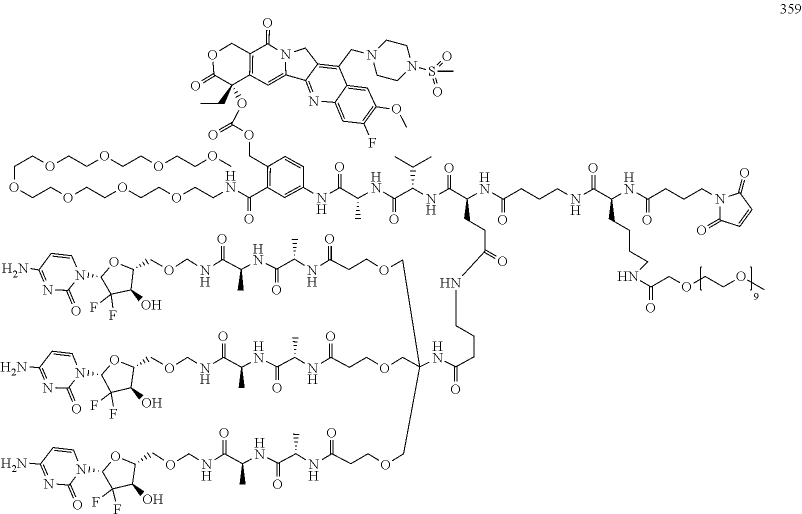

To a solution of compound 358 (186 mg, 0.060 mmol) and compound 60 (78 mg, 0.090 mmol) in DMF (5 mL) were added HATU (34 mg, 0.090 mmol) and DIPEA (16 mg, 0.121 mmol). The mixture was stirred at RT for 10 min and purified by prep-HPLC (eluted with CH₃CN/H₂O (0.05% FA)=38/62) to afford 359 (21 mg, 9% yield) as a white solid. MS ESI (m/z): calcd for C₁₇₀H₂₅₂F₇N₃₃O₆₃S [M/2+H]⁺: 1965.36; found 1966.58.

### Example 234: allyl ((2S)-1-(((2S)-1-((3-((2,5,8,11,14,17,20,23-octaoxapentacosan-25-yl) carbamoyl)-4-((((((1S,9S)-1-(2,2-dimethyl-1,3-dioxolane-4-carboxamido)-9-ethyl-5-fluoro-4-methyl-10,13-dioxo-2,3,9,10, 13,15-hexahydro-1H, 12H-benzo[de]pyrano[3′,4′: 6,7]indolizino[1,2-b]quinolin-9-yl)oxy)carbonyl)oxy)methyl)phenyl)amino)-1-oxopropan-2-yl)amino)-3-methyl-1-oxobutan-2-yl)carbamate (362)

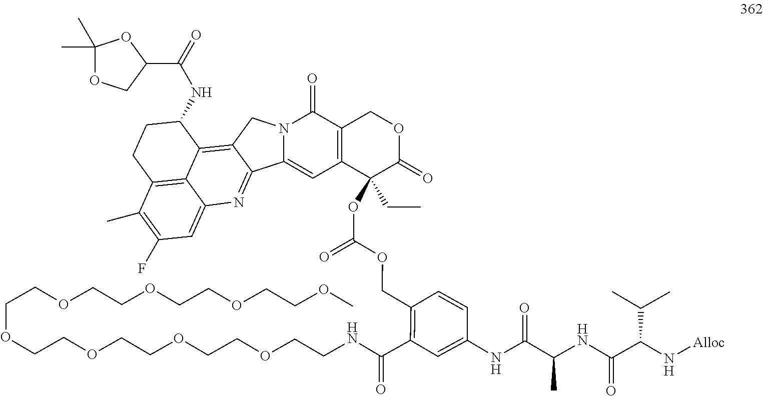

To a solution of compound 361 (400 mg, 0.710 mmol) in DCM (20 mL) were added triphosgene (126 mg, 0.426 mmol) and DMAP (347 mg, 2.840 mmol). The mixture was stirred at RT for 20 min and then compound 330 (1098 mg, 1.420 mmol) was added. After stirring at RT for 1 h, the reaction mixture was concentrated in vacuo. The residue was purified by silica gel column chromatography (eluted with DCM/MeOH=20/1) to afford 362 (850 mg, 87% yield) as a white solid. MS ESI (m/z): calcd for C₆₈H₉₀FN₇O₂₂ [M+H]⁺: 1376.62; found 1376.96.

### Example 235: 2-((2,5,8,11,14,17,20,23-octaoxapentacosan-25-yl)carbamoyl)-4-((S)-2-((S)-2-amino-3-methylbutanamido) propanamido)benzyl ((1S,9S)-1-(2,2-dimethyl-1,3-dioxolane-4-carboxamido)-9-ethyl-5-fluoro-4-methyl-10,13-dioxo-2,3,9,10,13,15-hexahydro-1H, 12H-benzo[de] pyrano[3′,4′: 6,7]indolizino[1,2-b]quinolin-9-yl)carbonate (363)

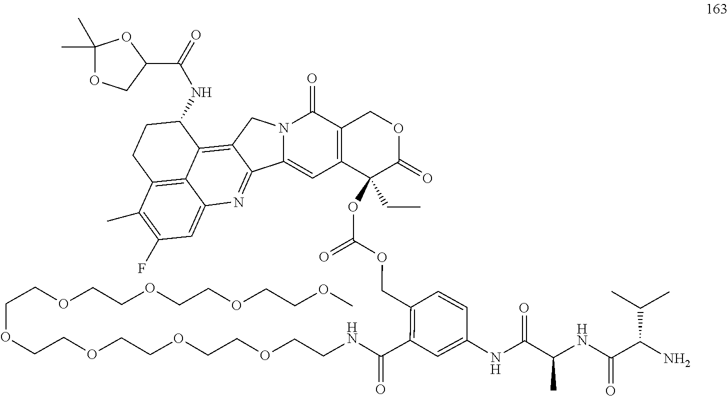 [SMILES: CC[C@@]1(OC(=O)OCc2ccc(NC(=O)[C@H](C)NC(=O)[C@@H](N)C(C)C)cc2C(=O)NCCOCCOCCOCCOCCOCCOCCOCCOC)C(=O)OCc2c1cc1n(c2=O)Cc2c-1nc1cc(F)c(C)c3c1c2[C@@H](NC(=O)C1COC(C)(C)O1)CC3]

To a solution of compound 362 (850 mg, 0.618 mmol) in DCM (5 mL) was added Pd (PPh₃)₄ (36 mg, 0.031 mmol), followed by pyrrolidine (279 mg, 1.235 mmol). The mixture was stirred at RT for 0.5 h, and then concentrated in vacuo. The residue was purified by silica gel column chromatography (eluted with DCM/MeOH=15/1) to afford 363 (405 mg, 51% yield) as a white solid. MS ESI (m/z): calcd for C₆₄H₈₆FN₇O₂₀ [M+H]⁺: 1292.59; found 1292.47.

### Example 236: allyl (4S)-5-(((2S)-1-(((2S)-1-((3-((2,5,8,11,14,17,20,23-octaoxapentacosan-25-yl) carbamoyl)-4-((((((1S,9S)-1-(2,2-dimethyl-1,3-dioxolane-4-carboxamido)-9-ethyl-5-fluoro-4-methyl-10,13-dioxo-2,3,9,10, 13,15-hexahydro-1H,12H-benzo[de]pyrano[3′,4′: 6,7]indolizino[1,2-b]quinolin-9-yl)oxy)carbonyl)oxy)methyl)phenyl)amino)-1-oxopropan-2-yl)amino)-3-methyl-1-oxobutan-2-yl)amino)-4-((((9H-fluoren-9-yl)methoxy)carbonyl)amino)-5-oxopentanoate (364)

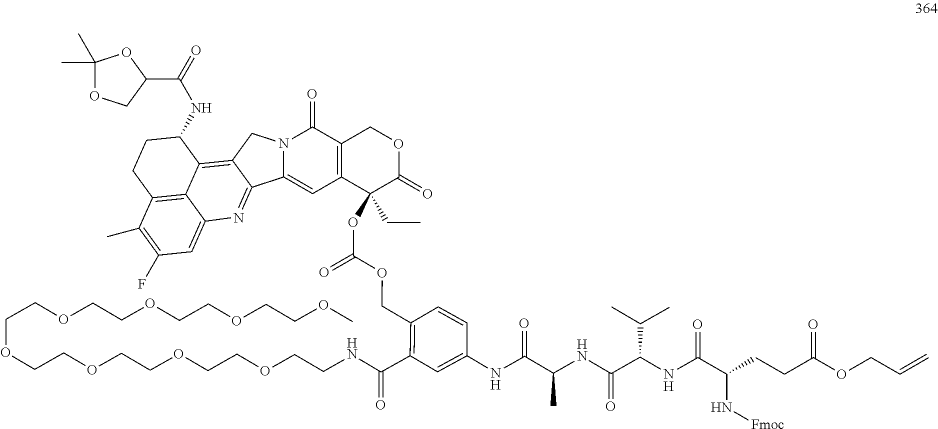

To a solution of compound 363 (405 mg, 0.313 mmol) in DMF (5 mL) was added HATU (131 mg, 0.345 mmol). After stirring at RT for 5 min, compound 133 (141 mg, 0.345 mmol) and DIPEA (81 mg, 0.627 mmol) were added. The mixture was stirred at RT for 10 min and concentrated in vacuo. The residue was purified by silica gel column chromatography (eluted with DCM/MeOH=94/6) to afford 364 (530 mg, 100% yield) as a white solid. MS ESI (m/z): calcd for C₈₇H₁₀₇FN₈O₂₅ [M+H]⁺: 1683.73; found 1685.15.

### Example 237: (4S)-5-(((2S)-1-(((2S)-1-((3-((2,5,8,11,14,17,20,23-octaoxapentacosan-25-yl) carbamoyl)-4-((((((1S,9S)-1-(2,2-dimethyl-1,3-dioxolane-4-carboxamido)-9-ethyl-5-fluoro-4-methyl-10,13-dioxo-2,3,9,10, 13,15-hexahydro-1H,12H-benzo[de]pyrano[3′,4′: 6,7]indolizino[1,2-b]quinolin-9-yl)oxy)carbonyl)oxy)methyl)phenyl)amino)-1-oxopropan-2-yl)amino)-3-methyl-1-oxobutan-2-yl) amino)-4-amino-5-oxopentanoic acid (365)

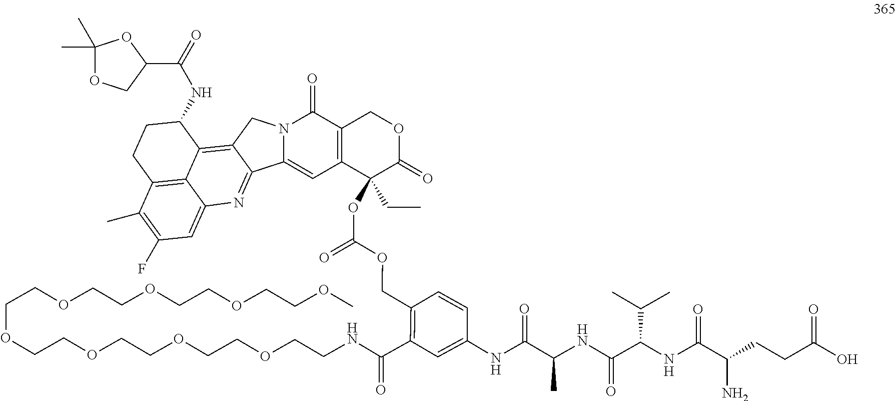 [SMILES: CC[C@@]1(OC(=O)OCc2ccc(NC(=O)[C@H](C)NC(=O)[C@@H](NC(=O)[C@@H](N)CCC(=O)O)C(C)C)cc2C(=O)NCCOCCOCCOCCOCCOCCOCCOCCOC)C(=O)OCc2c1cc1n(c2=O)Cc2c-1nc1cc(F)c(C)c3c1c2[C@@H](NC(=O)C1COC(C)(C)O1)CC3]

To a solution of compound 364 (546 mg, 0.324 mmol) in DCM (5 mL) was added Pd (PPh₃)₄ (19 mg, 0.016 mmol), then pyrrolidine (265 mg, 0.648 mmol). The mixture was stirred at RT for 0.5 h, and then concentrated in vacuo to afford 365 (270 mg, 59% yield) as a yellow solid. MS ESI (m/z): calcd for C₆₉H₉₃FN₈O₂₃ [M+H]⁺: 1421.63; found 1421.52.

### Example 238: (37S,45S)-45-(((2S)-1-(((2R)-1-((3-((2,5,8,11,14,17,20,23-octaoxapentacosan-25-yl)carbamoyl)-4-((((((1S,9S)-1-(2,2-dimethyl-1,3-dioxolane-4-carboxamido)-9-ethyl-5-fluoro-4-methyl-10,13-dioxo-2,3,9,10,13,15-hexahydro-1H, 12H-benzo[de]pyrano[3′,4′: 6,7]indolizino[1,2-b]quinolin-9-yl)oxy)carbonyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8, 11,14,17,20,23,26,29-decaoxa-32,39,44-triazaoctatetracontan-48-oic acid (366)

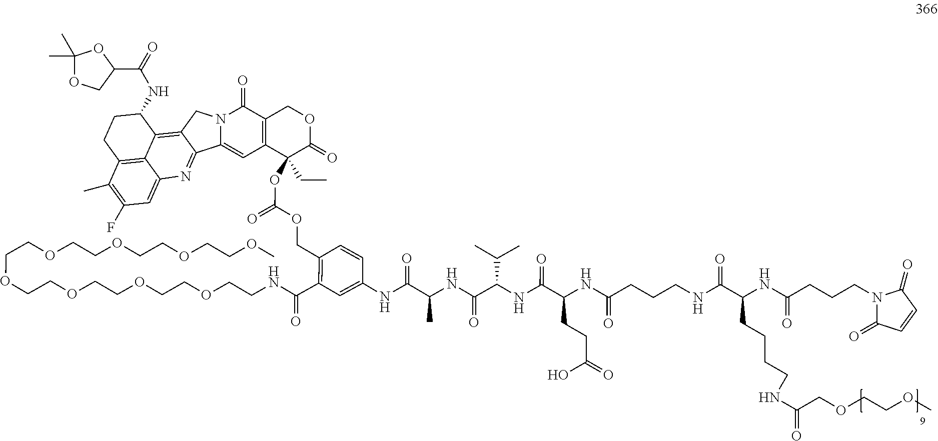

To a solution of compound 365 (200 mg, 0.141 mmol) and compound 117 (145 mg, 0.141 mmol) in DMF (2 mL) was added DIPEA (36 mg, 0.282 mmol). The mixture was stirred at RT for 1 h, concentrated under reduced pressure to give 366 (45 mg, 14% yield) as a yellow solid. ESI: m/z: calcd for C₁₀₈H₁₅₉FN₁₂O₃₉ [M+H]⁺: 2268.08; found 2269.55.

### Example 239: 2-((2,5,8,11,14,17,20,23-octaoxapentacosan-25-yl)carbamoyl)-4-((37S,45S,48S,51R)-45-((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-6,9-dimethyl-5,8,11-trioxo-2,14-dioxa-4,7,10-triazapentadecan-15-yl)-6,9-dimethyl-5,8,11,18,23-pentaoxo-2,14-dioxa-4,7,10,17,22-pentaazapentacosan-25-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-1-(2,2-dimethyl-1,3-dioxolane-4-carboxamido)-9-ethyl-5-fluoro-4-methyl-10,13-dioxo-2,3,9,10,13,15-hexahydro-1H, 12H-benzo[de]pyrano[3′,4′: 6,7]indolizino[1,2-b]quinolin-9-yl)carbonate (367)

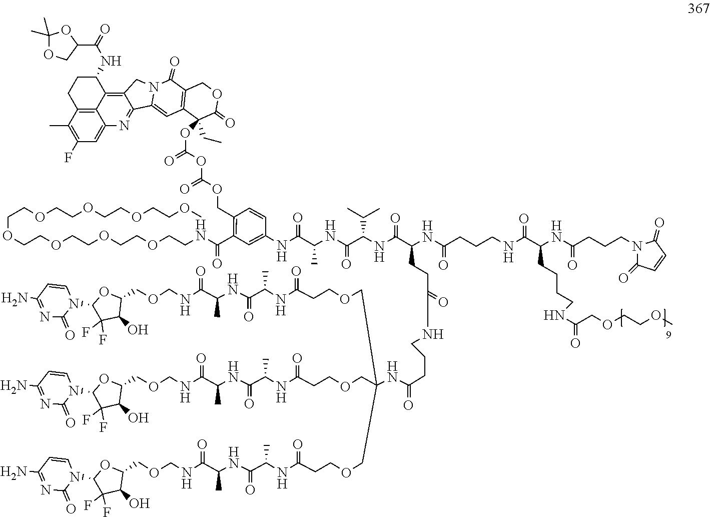

To a solution of compound 366 (45 mg, 0.020 mmol), compound 290 (50 mg, 0.030 mmol) and HATU (8 mg, 0.021 mmol) in DMF (1 mL) was added DIEA (8 mg, 0.060 mmol). The reaction mixture was stirred at room temperature for 1 h, and then concentrated under reduced pressure to give 367 (25 mg, 32% yield) as a white solid. ESI MS m/z: calcd for C₁₇₃H₂₅₃F₇N₃₂O₆₃ [M+2H]₂₊: 1960.88; found 1961.53.

### Example 240: Synthesis of benzyl ((S)-1-(((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl) amino)-3-methyl-1-oxobutan-2-yl)carbamate (370)

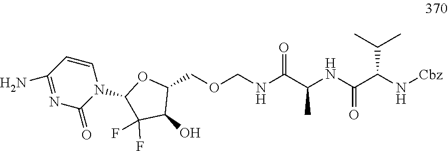 [SMILES: CC(C)[C@H](NC(=O)OCc1ccccc1)C(=O)N[C@@H](C)C(=O)NCOC[C@H]1O[C@@H](n2ccc(N)nc2=O)C(F)(F)[C@@H]1O]

To a solution of compound 285 (2.70 g, 7.431 mmol) and 202 (1.87 g, 7.431 mmol) and HATU (2.97 g, 7.803 mmol) in DMF (20 mL) was added DIEA (1.44 g, 11.147 mmol). The reaction mixture was stirred at room temperature for 20 min and then concentrated under reduced pressure. The resulting residue was purified by flash column chromatography (DCM:MeOH=85:15) to afford 370 (3.60 g, 81% yield) as a yellow solid. ESI MS m/z: calcd for C₂₆H₃₅F₂N₆O₈ [M+H]⁺: 597.24; found 597.15.

### Example 241: (S)-2-amino-N-((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl)-3-methylbutanamide (371)

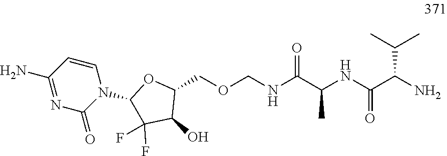 [SMILES: CC(C)[C@H](N)C(=O)N[C@@H](C)C(=O)NCOC[C@H]1O[C@@H](n2ccc(N)nc2=O)C(F)(F)[C@@H]1O]

To a solution of compound 370 (3.60 g, 6.034 mmol) in MeOH (40 mL) at room temperature was added Pd/C (0.36 g, 10 wt %). The resulting mixture was stirred at room temperature under a H₂ balloon for 7 h before it was filtered through a pad of Celite. The filtrate was concentrated under reduced pressure to afford 371 (3.00 g, crude) as a yellow solid. ESI MS m/z: calcd for C₁₈H₂₉F₂N₆O₆ [M+H]⁺: 463.20; found 463.09.

### Example 242: benzyl ((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2,14-dioxa-4,7,10-triazapentadecan-15-yl)-9-isopropyl-6-methyl-5,8,11,18-tetraoxo-2,14-dioxa-4,7,10,17-tetraazahenicosan-21-yl)carbamate (372)

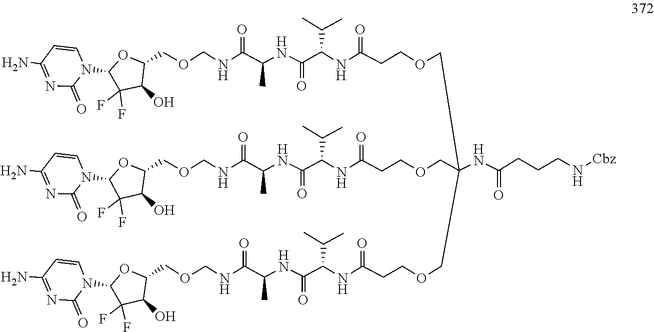 [SMILES: CC(C)[C@H](NC(=O)CCOCC(COCCC(=O)N[C@H](C(=O)N[C@@H](C)C(=O)NCOC[C@H]1O[C@@H](n2ccc(N)nc2=O)C(F)(F)[C@@H]1O)C(C)C)(COCCC(=O)N[C@H](C(=O)N[C@@H](C)C(=O)NCOC[C@H]1O[C@@H](n2ccc(N)nc2=O)C(F)(F)[C@@H]1O)C(C)C)NC(=O)CCCNC(=O)OCc1ccccc1)C(=O)N[C@@H](C)C(=O)NCOC[C@H]1O[C@@H](n2ccc(N)nc2=O)C(F)(F)[C@@H]1O]

To a solution of compound 283 (95 mg, 0.171 mmol) and compound 371 (300 mg, 0.649 mmol) and HATU (208 mg, 0.546 mmol) in DMF (3 mL) was added DIEA (88 mg, 0.683 mmol). The reaction mixture was stirred at room temperature for 30 min and concentrated under reduced pressure. The resulting residue was purified by prep-HPLC to afford 372 (160 mg, 49% yield) as a white solid. ESI MS m/z: calcd for C₇₉H₁₁₅F₆N₂₀O₂₇ [M+H]⁺: 1889.81; found 1889.81.

### Example 243: ((6S,9S)-1-((2R,3R,5R)-5-(4-amino-2-hydroxypyrimidin-1 (2H)-yl)-4,4-difluoro-3-hydroxy-tetrahydrofuran-2-yl)-16,16-bis((6S,9S)-1-((2R,3R,5R)-5-(4-amino-2-hydroxypyrimidin-1 (2H)-yl)-4,4-difluoro-3-hydroxy-tetrahydrofuran-2-yl)-9-isopropyl-6-methyl-5,8,11-trioxo-2,14-dioxo-4,7,10-triazapentadecane-15-yl)-9-isopropyl-6-methyl-5,8,11,18-tetraoxo-2,14-dioxo-4,7,10,17-tetrazahexadecane-21-yl)amine (373)

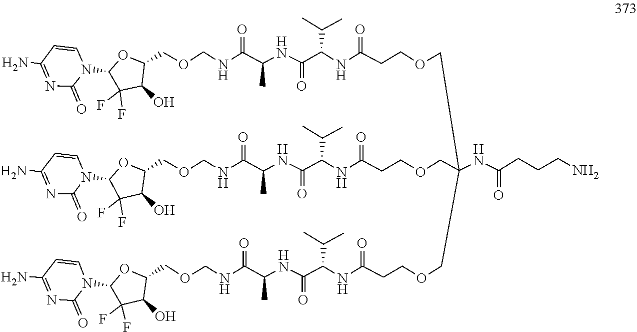 [SMILES: CC(C)[C@H](NC(=O)CCOCC(COCCC(=O)N[C@H](C(=O)N[C@@H](C)C(=O)NCOC[C@H]1O[C@@H](n2ccc(N)nc2=O)C(F)(F)[C@@H]1O)C(C)C)(COCCC(=O)N[C@H](C(=O)N[C@@H](C)C(=O)NCOC[C@H]1O[C@@H](n2ccc(N)nc2=O)C(F)(F)[C@@H]1O)C(C)C)NC(=O)CCCN)C(=O)N[C@@H](C)C(=O)NCOC[C@H]1O[C@@H](n2ccc(N)nc2=O)C(F)(F)[C@@H]1O]

To a solution of compound 372 (160 mg, 0.085 mmol) in MeOH (4 mL) at room temperature were added Pd/C (18 mg, 10 wt %) and NH₃/MeOH (0.5 mL). The resulting mixture was stirred at 50° C. under a H₂ balloon for 1.5 h before it was filtered through a pad of Celite. The filtrate was concentrated under reduced pressure to afford 373 (128 mg, 86% yield) as a white solid. ESI MS m/z: calcd for C₇₁H₁₀₉F₆N₂₀O₂₅ [M+H]⁺: 1755.77; found 1756.06.

### Example 244: 2-((2,5,8,11,14,17,20,23-octaoxapentacosan-25-yl)carbamoyl)-4-((37S,45S,48S,51S)-45-((6S,9S)-1-((2R,3R,5R)-5-(4-amino-2-oxopyrimidin-1 (2H)-yl)-4,4-difluoro-3-hydroxytetrahydrofuran-2-yl)-16,16-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2,14-dioxa-4,7,10-triazapentadecan-15-yl)-9-isopropyl-6-methyl-5,8,11,18,23-pentaoxo-2,14-dioxa-4,7,10,17,22-pentaazapentacosan-25-yl)-37-(4-(2,5-dioxo-2,5-dihydro-1H-pyrrol-1-yl)butanamido)-48-isopropyl-51-methyl-31,38,43,46,49-pentaoxo-2,5,8,11, 14, 17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (374)

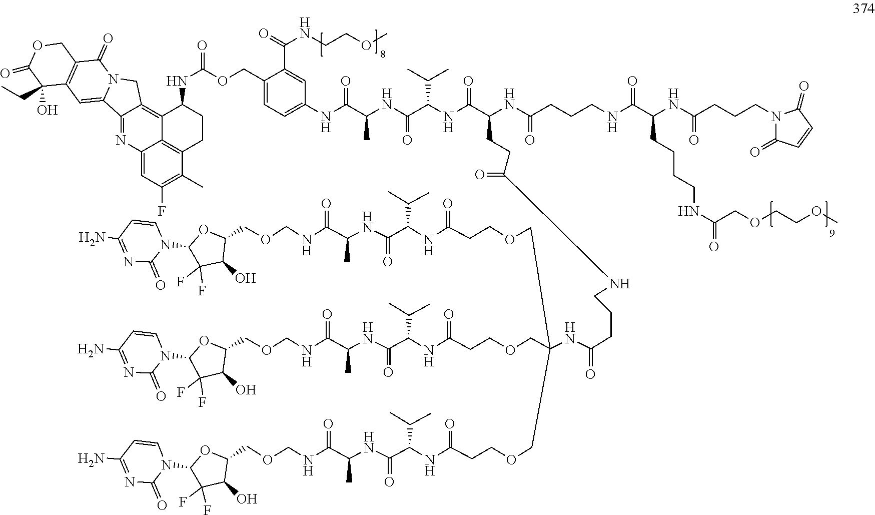

To a solution of compound 373 (112 mg, 0.053 mmol) in DMF (3 mL) was added HATU (21 mg, 0.055 mmol) at room temperature. The mixture was stirred at room temperature for 10 min and then compound 291 (250 mg, 0.150 mmol) and DIEA (10 mg, 0.079 mmol) were added. The reaction mixture was stirred at room temperature for 0.5 h, and quenched by formic acid (0.5 mL), purified by prep-HPLC to afford 374 (109 mg, 45% yield) as a white solid. ESI MS m/z: calcd for C₁₇₃H₂₅₇F₇N₃₂O₆₀ [M/2+H]⁺: 1938.90; found 1939.73.

### Example 245: di-tert-butyl 22-(4-(((benzyloxy)carbonyl)amino) butanamido)-22-(23,23-dimethyl-5,21-dioxo-2,9,12,15,18,22-hexaoxa-6-azatetracosyl)-17,27-dioxo-4,7, 10,13,20,24,31,34,37,40-decaoxa-16,28-diazatritetracontanedioate (377)

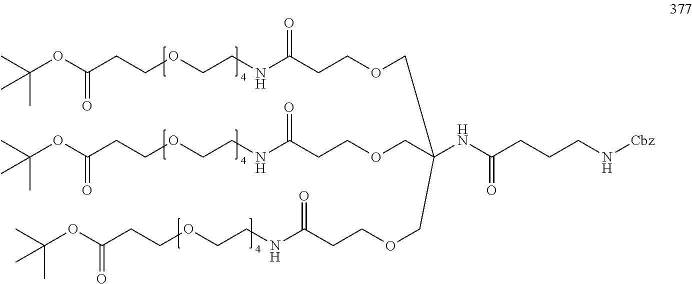

To a solution of compound 283 (1.75 g, 3.144 mmol), compound 376 (3.54 g, 11.005 mmol) and HATU (3.95 g, 10.376 mmol) in DMF (20 mL) was added DIEA (1.63 g, 12.557 mmol). The reaction mixture was stirred at room temperature for 1 h and then concentrated under reduced pressure. The resulting residue was diluted with DCM (40 mL), and then washed with saturated Na₂CO₃ (10 mL), concentrated under reduced pressure to afford 377 (4.65 g, 100% yield) as a yellow oil. ESI MS m/z: calcd for C₇₀H₁₂₄N₅O₂₇ [M+H]⁺: 1466.84; found 1466.97.

### Example 246: 22-(4-(((benzyloxy)carbonyl)amino) butanamido)-22-(20-carboxy-5-oxo-2,9,12,15,18-pentaoxa-6-azaicosyl)-17,27-dioxo-4,7,10,13,20,24,31,34,37,40-decaoxa-16,28-diazatritetracontanedioic acid (378)

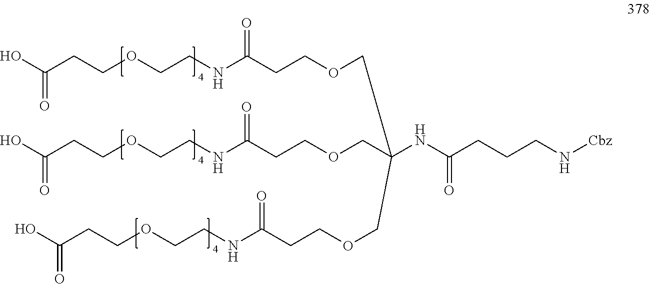

To a solution of compound 377 (4.65 g, 3.170 mmol) in DCM (40 mL) at room temperature was added TFA (30 mL). The resulting mixture was stirred at room temperature for 2 h before it was concentrated under reduced pressure. The resulting residue was purified by prep-HPLC to afford 378 (2.49 g, 60% yield) as a colorless oil. ESI MS m/z: calcd for C₅₈H₁₀₀N₅O₂₇ [M+H]⁺: 1298.65; found 1298.31.

### Example 247: benzyl ((6S,9S)-1-((2R,3R,5R)-5-(4-amino-2-oxopyrimidin-1 (2H)-yl)-4,4-difluoro-3-hydroxytetrahydrofuran-2-yl)-32,32-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,27-tetraoxo-2,14,17,20,23,30-hexaoxa-4,7,10,26-tetraazahentriacontan-31-yl)-9-isopropyl-6-methyl-5,8,11,27,34-pentaoxo-2,14,17,20,23,30-hexaoxa-4,7,10,26,33-pentaazaheptatriacontan-37-yl)carbamate (379)

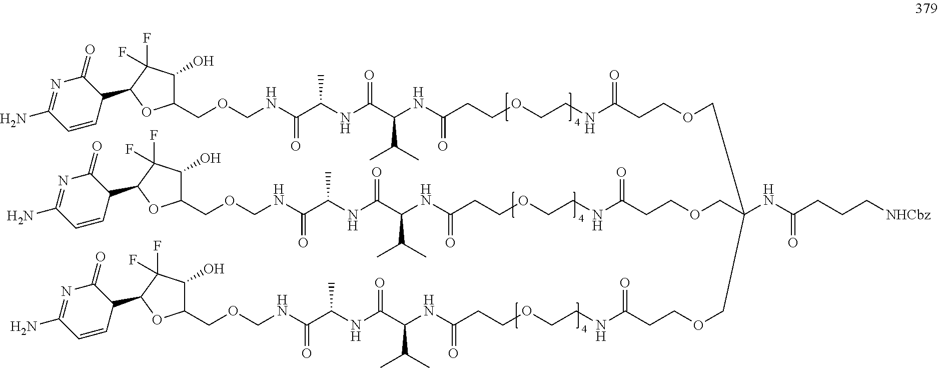

To a solution of compound 378 (1.80 g, 1.390 mmol), compound 371 (2.25 g, 4.865 mmol) and HATU (1.69 g, 4.448 mmol) in DMF (18 mL) was added DIEA (0.81 g, 6.256 mmol). The reaction mixture was stirred at room temperature for 30 min and then concentrated under reduced pressure. The resulting residue was purified by prep-HPLC to afford 379 (1.80 g, 49% yield) as a white solid. ESI MS m/z: calcd for C₁₁₂H₁₇₇F₆N₂₃O₄₂ [M+2H]₂₊: 1316.12 found; 1316.29.

### Example 248:1,1′-(((6S,9S)-1-((2R,3R,5R)-5-(4-amino-2-oxopyrimidin-1 (2H)-yl)-4,4-difluoro-3-hydroxytetrahydrofuran-2-yl)-32-(4-aminobutanamido)-9-isopropyl-6-methyl-5,8,11,27-tetraoxo-32-((3-oxopropoxy)methyl)-2,14,17,20,23,30,34-heptaoxa-4,7,10,26-tetraazaheptatriacontan-37-oyl)bis(azanediyl))bis(N-((S)-1-(((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl)amino)-3-methyl-1-oxobutan-2-yl)-3′,6′,9′,12′-tetraoxapentadecan-15-amide) (380)

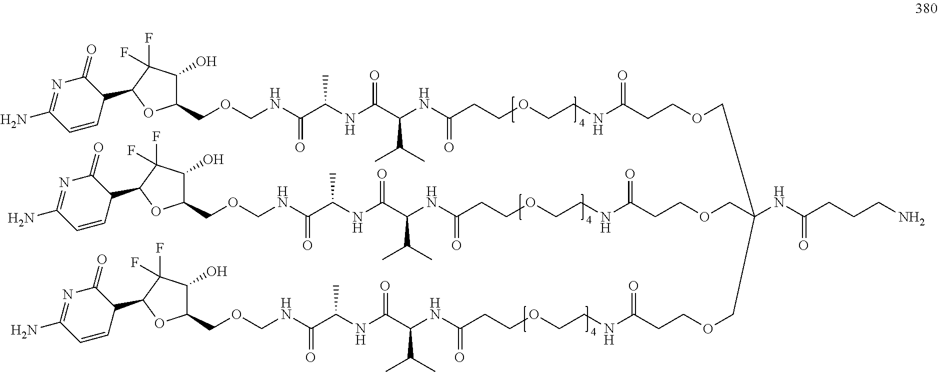

To a solution of compound 379 (1.53 g, 0.582 mmol) in MeOH (20 mL) at room temperature were added Pd/C (0.30 g, 10 wt %) and NH₃/MeOH (2.0 mL). The resulting mixture was stirred at 50° C. under a H₂ balloon for 1.5 h before it was filtered through a pad of Celite. The filtrate was concentrated under reduced pressure to afford 380 (1.40 g, 96% yield) as a white solid. ESI MS m/z: calcd for C₁₀₄H₁₇₁F₆N₂₃O₄₀ [M+2H]₂₊: 1249.10 found 1249.05.

### Example 249: 2-((2,5,8,11,14,17,20,23-octaoxapentacosan-25-yl)carbamoyl)-4-((37S,45S,48S,51S)-45-((6S,9S)-1-((2R,3R,5R)-5-(4-amino-2-oxopyrimidin-1 (2H)-yl)-4,4-difluoro-3-hydroxytetrahydrofuran-2-yl)-32,32-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,27-tetraoxo-2,14,17,20,23,30-hexaoxa-4,7,10,26-tetraazahentriacontan-31-yl)-9-isopropyl-6-methyl-5,8,11,27,34,39-hexaoxo-2,14,17,20,23,30-hexaoxa-4,7,10,26,33,38-hexaazahentetracontan-41-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (381)

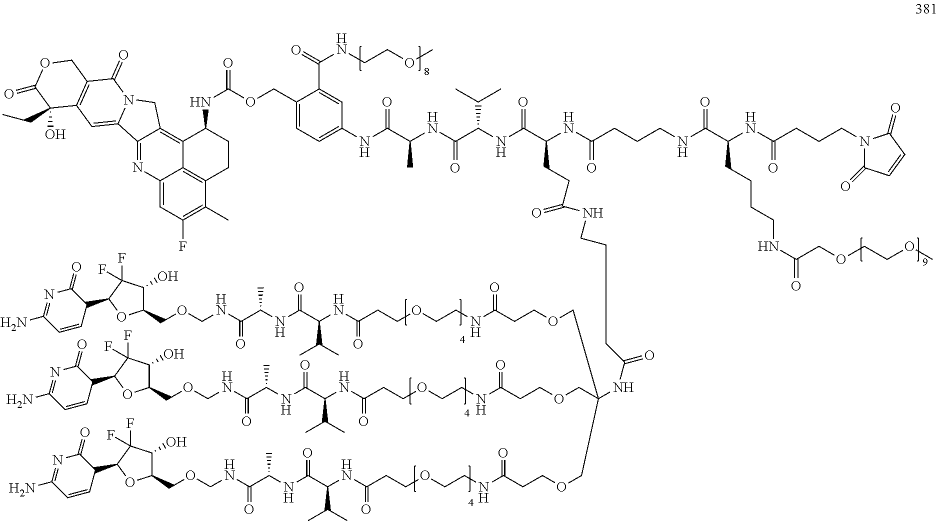

To a solution of compound 291 (112 mg, 0.053 mmol) in DMF (3 mL) was added HATU (21 mg, 0.055 mmol) at room temperature. The mixture was stirred at room temperature for 10 min and then compound 380 (131.0 mg, 0.052 mmol) and DIEA (10 mg, 0.079 mmol) were added. The reaction mixture was stirred at room temperature for 0.5 h, quenched by formic acid (0.5 mL), and purified by prep-HPLC to afford 381 (109 mg, 45% yield) as a white solid. ESI MS m/z: calcd for C₂₀₆H₃₂₀F₇N₃₅O₇₅ [M+2H]₂₊: 2309.61 found 2309.55.

### Example 250: 2-((2,5,8,11,14,17,20,23-octaoxapentacosan-25-yl)carbamoyl)-4-((6S,9S,70S, 73S, 76S)-1-((2R,3R,5R)-5-(4-amino-2-oxopyrimidin-1 (2H)-yl)-4,4-difluoro-3-hydroxytetrahydrofuran-2-yl)-32,32-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,27-tetraoxo-2,14,17,20,23,30-hexaoxa-4,7,10,26-tetraazahentriacontan-31-yl)-70-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-9,73-diisopropyl-6,76-dimethyl-5,8,11,27,34,39,67,71,74-nonaoxo-2,14,17,20,23,30,42,45,48,51,54,57,60,63-tetradecaoxa-4,7,10,26,33,38,66,72,75-nonaazaheptaheptacontan-77-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (384)

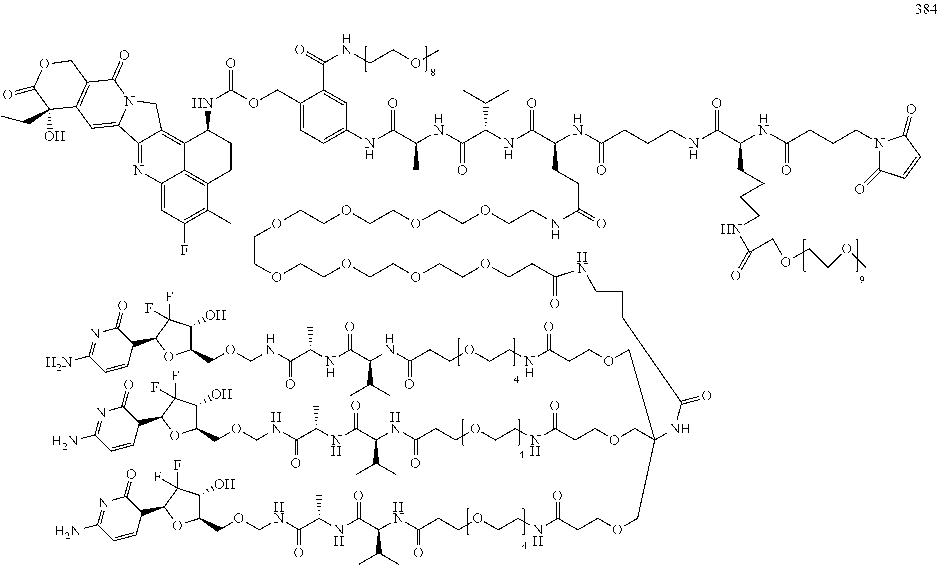

To a solution of compound 383 (215 mg, 0.084 mmol) in DMF (3 mL) was added HATU (34 mg, 0.0884 mmol) at room temperature. The mixture was stirred at room temperature for 10 min. Then compound 380 (209 mg, 0.084 mmol) and DIEA (16 mg, 0.126 mmol) were added and the reaction mixture was stirred at room temperature for 0.5 h, quenched by formic acid (0.5 mL), then purified by prep-HPLC to afford 384 (188 mg, 44% yield) as a white solid. ESI MS m/z: calcd for C₂₂₅H₃₅₇F₇N₃₆O₈₄ [M+4H]⁴⁺: 1261.12; found 1261.17.

### Example 251: 2-((2,5,8,11,14,17,20,23-octaoxapentacosan-25-yl)carbamoyl)-4-((6S,9S,70S, 73S, 76S)-1-((2R,3R,5R)-5-(4-amino-2-oxopyrimidin-1 (2H)-yl)-4,4-difluoro-3-hydroxytetrahydrofuran-2-yl)-32,32-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,27-tetraoxo-2, 14,17,20,23,30-hexaoxa-4,7,10,26-tetraazahentriacontan-31-yl)-70-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-9,73-diisopropyl-6,76-dimethyl-5,8,11,27,34,39,67,71,74-nonaoxo-2,14,17,20,23,30,42,45,48,51,54,57,60,63-tetradecaoxa-4,7,10,26,33,38,66,72,75-nonaazaheptaheptacontan-77-amido)benzyl ((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)carbamate (387)

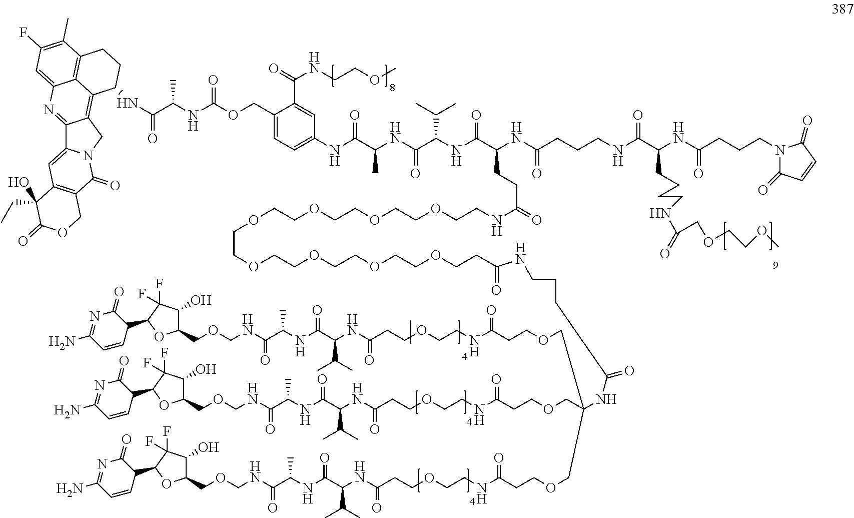

To a solution of compound 386 (242 mg, 0.092 mmol) in DMF (3 mL) was added HATU (37 mg, 0.096 mmol) at room temperature. The mixture was stirred at room temperature for 10 min. Then compound 380 (229 mg, 0.092 mmol) and DIEA (18 mg, 0.138 mmol) were added. The reaction mixture was stirred at room temperature for 0.5 h, quenched by formic acid (0.5 mL), and purified by prep-HPLC to afford 387 (173 mg, 36% yield) as a white solid. ESI MS m/z: calcd for C₂₂₈H₃₆₂F₇N₃₇O₈₅ [M/4+H]⁺: 1278.88; found 1278.94.

### Example 252: perfluorophenyl (37S,45S)-37-(4-(2,5-dioxo-2,5-dihydro-1H-pyrrol-1-yl) butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43-trioxo-2,5,8,11,14,17,20,23,26,29-decaoxa-32,39,44-triazaoctatetracontan-48-oate (389)

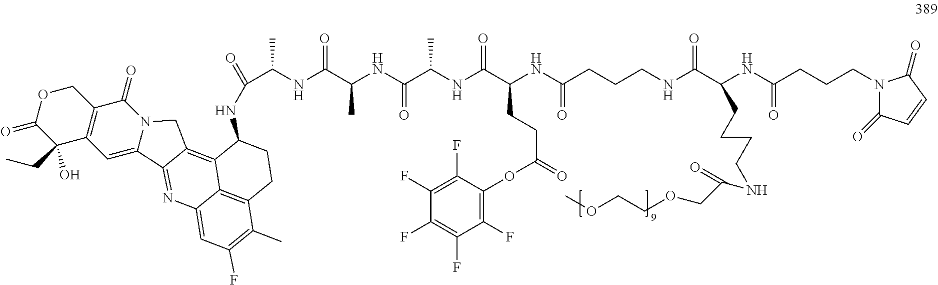

To a solution of compound 309 (340 mg, 0.209 mmol) in DCM (8 mL) were added PFP-OH (50 mg, 0.272 mmol) and EDCI (64 mg, 0.335 mmol). The mixture was stirred at room temperature for 2.0 h, and then washed with brine (5 mL×2), dried over Na₂SO₄, concentrated under reduced pressure to afford 389 (370 mg, 100% yield) as a yellow solid. ESI MS m/z: calcd for C₈₃H₁₀₉F₆N₁₁O₂₆ [M+H]⁺: 1790.74; found 1791.01.

### Example 253: (37S,45S)-37-(4-(2,5-dioxo-2,5-dihydro-1H-pyrrol-1-yl)butanamido)-45-(((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)carbamoyl)-31,38,43,48-tetraoxo-2,5,8,11,14,17,20,23,26,29,52,55,58,61,64,67,70,73-octadecaoxa-32,39,44,49-tetraazahexaheptacontan-76-oic acid (391)

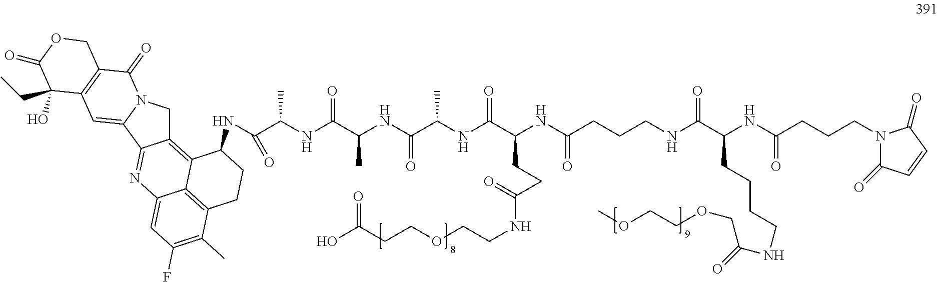

To a solution of compound 389 (350 mg, 0.195 mmol) in DMF (5 mL) were added compound 390 (104 mg, 0.235 mmol) and DIEA (38 mg, 0.293 mmol). The mixture was stirred at room temperature for 1.0 h, quenched by formic acid (0.5 mL), and purified by prep-HPLC to afford 391 (390 mg, 97% yield) as a yellow solid. ESI MS m/z: calcd for C₉₆H₁₄₇FN₁₂O₃₅ [M+H]⁺: 2048.01; found 2048.60.

### Example 254: (S)-N5-((6S,9S)-1-((2R,3R,5R)-5-(4-amino-2-oxopyrimidin-1 (2H)-yl)-4,4-difluoro-3-hydroxytetrahydrofuran-2-yl)-32,32-bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,27-tetraoxo-2,14,17,20,23,30-hexaoxa-4,7,10,26-tetraazahentriacontan-31-yl)-9-isopropyl-6-methyl-5,8,11,27,34,39-hexaoxo-2,14,17,20,23,30,42,45,48,51,54,57,60,63-tetradecaoxa-4,7,10,26,33,38-hexaazapentahexacontan-65-yl)-2-((S)-37-(4-(2,5-dioxo-2,5-dihydro-1H-pyrrol-1-yl)butanamido)-31,38-dioxo-2,5,8,11,14,17,20,23,26,29-decaoxa-32,39-diazatritetracontan-43-amido)-N1-((S)-1-(((S)-1-(((S)-1-(((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1-oxopropan-2-yl)amino)-1-oxopropan-2-yl)amino)-1-oxopropan-2-yl)pentanediamide (392)

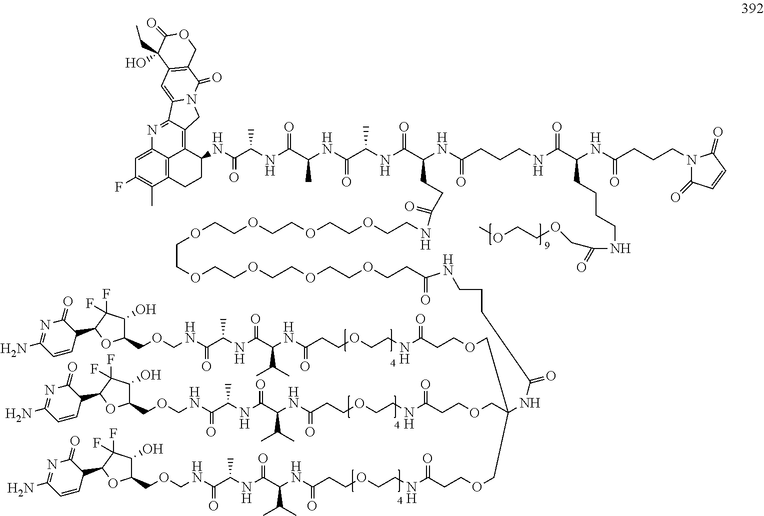

To a solution of compound 391 (173 mg, 0.085 mmol) in DMF (3 mL) was added HATU (34 mg, 0.089 mmol) at room temperature. The mixture was stirred at room temperature for 10 min. And then compound 380 (180 mg, 0.085 mmol) and DIEA (34 mg, 0.089 mmol) were added. The reaction mixture was stirred at room temperature for 0.5 h, quenched by formic acid (0.5 mL), and purified by prep-HPLC to afford 392 (140 mg, 36% yield) as a white solid. ESI MS m/z: calcd for C₂₀₀H₃₁₆F₇N₃₅O₇₄ [M+3H]³⁺: 1509.40; found 1509.39.

### Example 255: 2-((2,5,8,11,14,17,20,23-octaoxapentacosan-25-yl)carbamoyl)-4-((37S,45S,48S,51S)-45-((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11,39-tetraoxo-2,14,17,20,23,26,29,32,35-nonaoxa-4,7,10,38-tetraazahentetracontan-41-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (394)

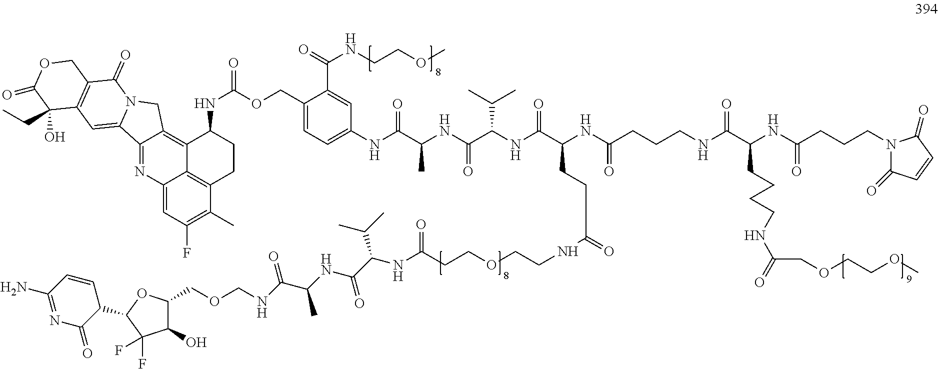

To a solution of compound 383 (280 mg, 0.109 mmol) in DMF (3 mL) was added HATU (44 mg, 0.114 mmol) at room temperature. The mixture was stirred at room temperature for 10 min. And then compound 371 (51 mg, 0.109 mmol) and DIEA (21 mg, 0.164 mmol) were added. The reaction mixture was stirred at room temperature for 0.5 h, quenched by formic acid (0.5 mL), and purified by prep-HPLC to afford 394 (65 mg, 19% yield) as a white solid. ESI MS m/z: calcd for C₁₃₉H₂₁₄F₃N₁₉O₅₀ [M+2H]₂₊: 1504.24; found 1504.21.

### Example 256: (2-(((benzyloxy)carbonyl)amino) acetamido) methyl acetate (397)

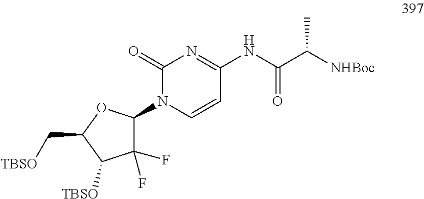 [SMILES: C[C@H](NC(=O)OC(C)(C)C)C(=O)Nc1ccn([C@@H]2O[C@H](CO[Si](C)(C)C(C)(C)C)[C@@H](O[Si](C)(C)C(C)(C)C)C2(F)F)c(=O)n1]

To a solution of compound 251 (300 mg, 0.610 mmol) and compound 396 (115 mg, 0.610 mmol) in CH₃CN (5 mL) were added TCFH (205 mg, 0.732 mmol) and NMI (100 mg, 1.22 mmol). The mixture was stirred at RT for 30 min and then concentrated in vacuo. The residue was purified by silica gel column chromatography (eluted with DCM/MeOH=50/1) to afford 397 (400 mg, 100% yield) as a white solid. MS ESI (m/z): calcd for C₂₉H₅₂F₂N₄OSi₂ [M+H]⁺: 663.34; found 662.89.

### Example 257: (S)-2-amino-N-(1-((2R,4R,5R)-4-((tert-butyldimethylsilyl)oxy)-3,3-difluoro-5-(hydroxymethyl)tetrahydrofuran-2-yl)-2-oxo-1,2-dihydropyrimidin-4-yl) propanamide (398)

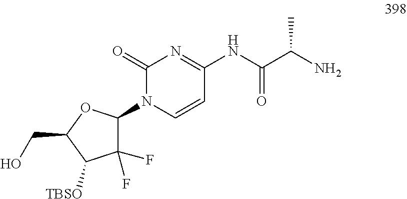 [SMILES: C[C@H](N)C(=O)Nc1ccn([C@@H]2O[C@H](CO)[C@@H](O[Si](C)(C)C(C)(C)C)C2(F)F)c(=O)n1]

To a solution of compound 397 (400 mg, 0.603 mmol) in CH₃CN (4 mL) was added TMSI (483 mg, 2.412 mmol) dropwise. The mixture was stirred at RT for 0.5 h and then quenched with H₂O (22 mg, 1.206 mmol), and DIPEA (233 mg, 1.809 mmol). The mixture was concentrated in vacuo to afford 398 (270 mg, crude) as a yellow solid. MS ESI (m/z): calcd for C₁₈H₃₀F₂N₄O₅Si [M+H]⁺: 449.21; found 448.85.

### Example 258: tert-butyl (4-(((S)-1-((1-((2R,4R,5R)-4-((tert-butyldimethylsilyl)oxy)-3,3-difluoro-5-(hydroxymethyl)tetrahydrofuran-2-yl)-2-oxo-1,2-dihydropyrimidin-4-yl)amino)-1-oxopropan-2-yl)amino)-4-oxobutyl)carbamate (400)

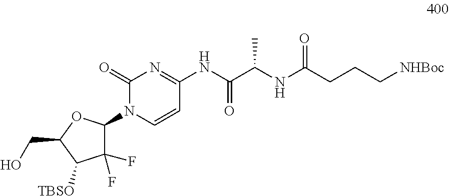 [SMILES: C[C@H](NC(=O)CCCNC(=O)OC(C)(C)C)C(=O)Nc1ccn([C@@H]2O[C@H](CO)[C@@H](O[Si](C)(C)C(C)(C)C)C2(F)F)c(=O)n1]

To a solution of compound 398 (270 mg, 0.602 mmol) and compound 399 (122 mg, 0.602 mmol) in DMF (5 mL) were added HATU (252 mg, 0.662 mmol) and DIPEA (156 mg, 1.204 mmol). The mixture was stirred at RT for 10 min and concentrated in vacuo. The residue was purified by silica gel column chromatography (eluted with DCM/MeOH=20/1) to afford 400 (180 mg, 47% yield) as a white solid. MS ESI (m/z): calcd for C₂₇H₄₅F₂N₅O₈Si [M+H]⁺: 634.31; found 633.97.

### Example 259:4-amino-N-((S)-1-((1-((2R,4R,5R)-3,3-difluoro-4-hydroxy-5-(hydroxymethyl) tetrahydrofuran-2-yl)-2-oxo-1,2-dihydropyrimidin-4-yl)amino)-1-oxopropan-2-yl)butanamide (401)

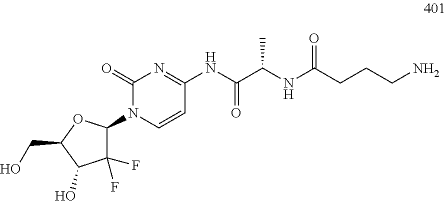 [SMILES: C[C@H](NC(=O)CCCN)C(=O)Nc1ccn([C@@H]2O[C@H](CO)[C@@H](O)C2(F)F)c(=O)n1]

To a solution of compound 400 (80 mg, 0.284 mmol) in CH₃CN (4 mL) was added TMSI (227 mg, 1.136 mmol) dropwise. The mixture was stirred at RT for 1 h and quenched with H₂O (10 mg, 0.568 mmol), then DIPEA (110 mg, 0.852 mmol). The mixture was concentrated in vacuo to afford 401 (119 mg, crude) as a yellow solid. MS ESI (m/z): calcd for C₁₆H₂₃F₂N₅O₆ [M+H]⁺: 420.17; found 419.73.

### Example 260: tert-butyl ((S)-1-((1-((2R,4R,5R)-3,3-difluoro-4-hydroxy-5-(hydroxymethyl) tetrahydrofuran-2-yl)-2-oxo-1,2-dihydropyrimidin-4-yl)amino)-14,14-bis((3-((4-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-4-oxobutyl)amino)-3-oxopropoxy)methyl)-2-methyl-1,4,9,16-tetraoxo-12-oxa-3,8,15-triazanonadecan-19-yl)carbamate (402)

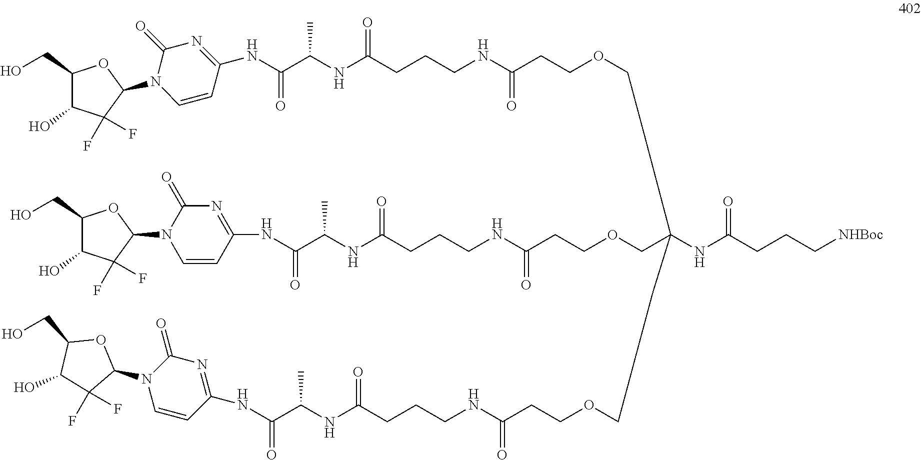 [SMILES: C[C@H](NC(=O)CCCNC(=O)CCOCCCCCC(CCCOCCC(=O)NCCCC(=O)N[C@@H](C)C(=O)Nc1ccn([C@@H]2O[C@H](CO)[C@@H](O)C2(F)F)c(=O)n1)(COCCC(=O)NCCCC(=O)N[C@@H](C)C(=O)Nc1ccn([C@@H]2O[C@H](CO)[C@@H](O)C2(F)F)c(=O)n1)NC(=O)CCCNC(=O)OC(C)(C)C)C(=O)Nc1ccn([C@@H]2O[C@H](CO)[C@@H](O)C2(F)F)c(=O)n1]

To a solution of compound 401 (119 mg, 0.284 mmol) and compound 283 (45 mg, 0.086 mmo) in DMF (5 mL) were added HATU (108 mg, 0.284 mmol) and DIPEA (67 mg, 0.517 mmol). The mixture was stirred at RT for 10 min and concentrated in vacuo. The residue was purified by prep-HPLC (eluted with CH₃CN/H₂O=30/70) to afford 402 (36 mg, 24% yield) as a white solid. MS ESI (m/z): calcd for C₇₀H₁₀₁F₆N₁₇O₂₇ [M+H]⁺: 1726.71; found 1727.22.

### Example 261:4,4′-(((S)-14-(4-aminobutanamido)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-2-methyl-1,4,9-trioxo-14-((3-oxopropoxy)methyl)-12,16-dioxa-3,8-diazanonadecan-19-oyl)bis(azanediyl))bis(N-((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)butanamide) (403)

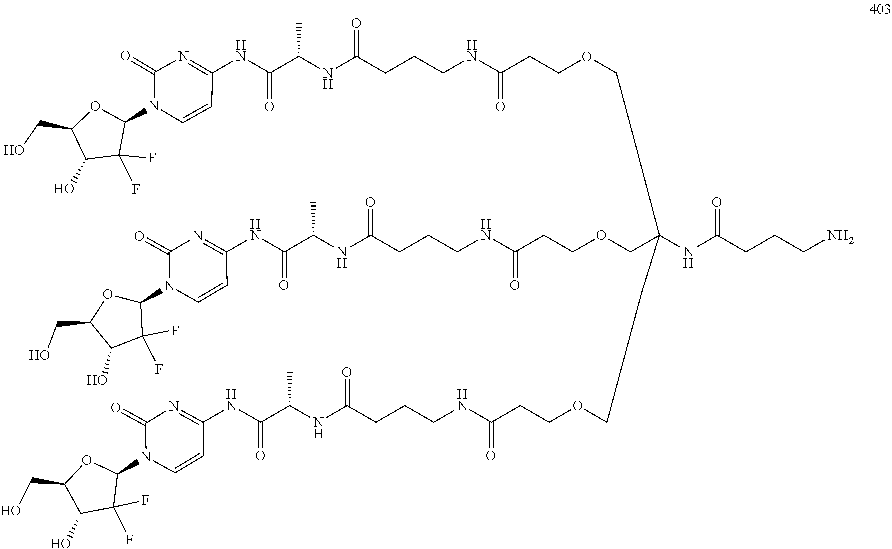 [SMILES: C[C@H](NC(=O)CCCNC(=O)CCOCCCCCC(CCCOCCC(=O)NCCCC(=O)N[C@@H](C)C(=O)Nc1ccn([C@@H]2O[C@H](CO)[C@@H](O)C2(F)F)c(=O)n1)(COCCC(=O)NCCCC(=O)N[C@@H](C)C(=O)Nc1ccn([C@@H]2O[C@H](CO)[C@@H](O)C2(F)F)c(=O)n1)NC(=O)CCCN)C(=O)Nc1ccn([C@@H]2O[C@H](CO)[C@@H](O)C2(F)F)c(=O)n1]

To a solution of compound 402 (36 mg, 0.021 mmol) in CH₃CN (5 mL) was added TMSI (25 mg, 0.126 mmol) dropwise. The mixture was stirred at RT for 1.5 h and then quenched with H₂O (1 mg, 0.042 mmol), diluted with EA (20 mL). The mixture was filtered and the cake was washed with EA to afford 403 (33 mg, crude) as a yellow solid. MS ESI (m/z): calcd for C₆₅H₉₃F₆N₁₇O₂₅ [M+H]⁺: 1626.65; found 1626.18.

### Example 262: 2-((2,5,8,11,14,17,20,23-octaoxapentacosan-25-yl)carbamoyl)-4-((37S,45S,48S,51S)-45-((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-14,14-bis((3-((4-(((S)-1-((1-((2R,4R,5R)-3,3-difluoro-4-hydroxy-5-(hydroxymethyl)tetrahydrofuran-2-yl)-2-oxo-1,2-dihydropyrimidin-4-yl)amino)-1-oxopropan-2-yl)amino)-4-oxobutyl)amino)-3-oxopropoxy)methyl)-2-methyl-1,4,9,16,21-pentaoxo-12-oxa-3,8,15,20-tetraazatricosan-23-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (404)

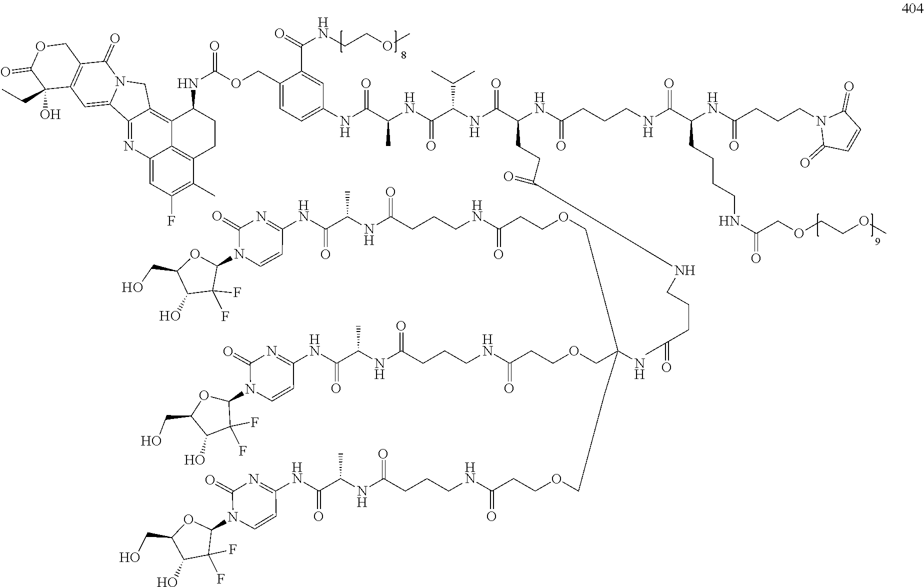

To a solution of compound 403 (34 mg, 0.021 mmol) and compound 291 (45 mg, 0.021 mmol) in DMF (5 mL) were added HATU (9 mg, 0.023 mmol) and DIPEA (5 mg, 0.042 mmol). The mixture was stirred at RT for 10 min and concentrated in vacuo to afford 404 (39 mg, 50% yield) as a white solid. MS ESI (m/z): calcd for C₁₆₇H₂₄₂F₇N₂₉O₆₀ [M+2H]₂₊: 1874.34; found 1874.37.

### Example 263: (((9H-fluoren-9-yl)methoxy)carbonyl)-L-valine-CTC resin (408)

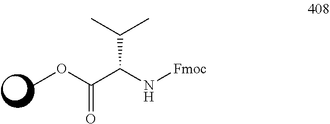

A solid phase peptide synthesis vessel (Chemgalss, 500-mL) were charged with 2-chlorotrityl chloride resin (8.00 g, 1.5 mmol/g) and DCM (80 mL). The mixture was stirred at RT for 2 h and then filtered. The resin was washed with DCM (80 mL), then a solution of compound 407 (4.07 g, 12.000 mmol) and DIPEA (3.10 g, 24.000 mmol) in anhydrous DCM (80 mL) was added. The reaction mixture was run overnight and the resin was washed with DCM (3×50 mL) to afford 408.

### Example 264: (1-(9H-fluoren-9-yl)-3-oxo-2,7,10-trioxa-4-azatridecan-13-oyl)-L-valine-CTC resin (410)

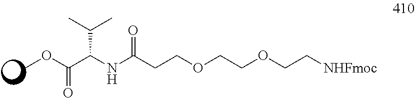

A solid phase peptide synthesis vessel (Chemgalss, 500-mL) was charged with 408. Fmoc deprotection was performed using piperidine (4.08 g, 48 mmol) in DMF (80 mL) for 2 h. The resin was then washed with DMF (3×80 mL), a solution of compound 409 (4.79 g, 12.000 mmol), HOBt (2.43 g, 18.000 mmol) and DIC (2.27 g, 18.000 mmol) in DMF (80 mL) was added. The reaction mixture was run for 2 h and the resin was washed with DMF (3×80 mL) to afford 410.

### Example 265: ((S)-7-((((9H-fluoren-9-yl)methoxy)carbonyl)amino)-2,2-dimethyl-4,8-dioxo-3,12,15-trioxa-9-azaoctadecan-18-oyl)-L-valine-CTC resin (412)

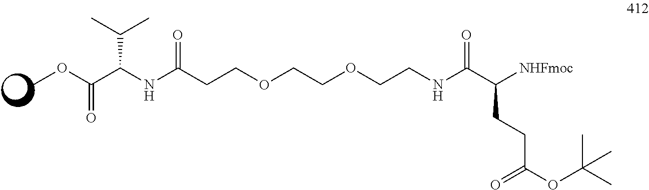

A solid phase peptide synthesis vessel (Chemgalss, 500-mL) was charged with 410. Fmoc deprotection was performed using piperidine (4.08 g, 48.000 mmol) in DMF (80 mL) for 2 h. The resin was then washed with DMF (3×80 mL), a solution of compound 411 (5.11 g, 12.000 mmol), HOBt (2.43 g, 18.000 mmol) and DIC (2.27 g, 18.000 mmol) in DMF (80 mL) was added. The reaction mixture was run for 2 h and the resin was washed with DMF (3×80 mL) to afford 412.

### Example 266: ((S)-7-(1-(9H-fluoren-9-yl)-3-oxo-2,7,10-trioxa-4-azatridecan-13-amido)-2,2-dimethyl-4,8-dioxo-3,12,15-trioxa-9-azaoctadecan-18-oyl)-L-valine-CTC resin (413)

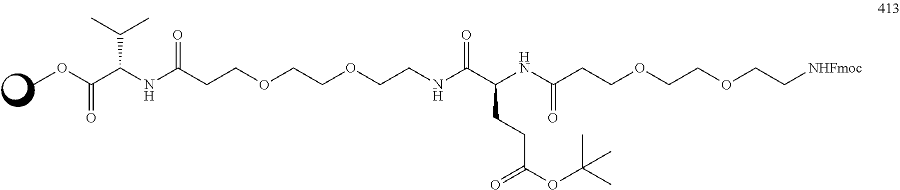

A solid phase peptide synthesis vessel (Chemgalss, 100-mL) was charged with 412 (6 mmol). Fmoc deprotection was performed using piperidine (2.04 g, 24.000 mmol) in DMF (40 mL) for 2 h. The resin was then washed with DMF (3×40 mL), and a solution of compound 409 (2.55 g, 6.000 mmol), HOBt (1.22 g, 9.000 mmol) and DIC (1.14 g, 9.000 mmol) in DMF (40 mL) was added. The reaction mixture was run for 2 h and the resin was washed with DMF (3×40 mL) to afford 413.

### Example 267: ((S)-7-((S)-7-((((9H-fluoren-9-yl)methoxy)carbonyl)amino)-2,2-dimethyl-4,8-dioxo-3,12,15-trioxa-9-azaoctadecan-18-amido)-2,2-dimethyl-4,8-dioxo-3,12,15-trioxa-9-azaoctadecan-18-oyl)-L-valine-CTC resin (414)

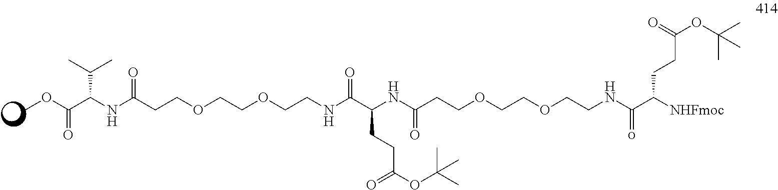

A solid phase peptide synthesis vessel (Chemgalss, 100-mL) was charged with 413. Fmoc deprotection was performed using piperidine (2.04 g, 24.000 mmol) in DMF (40 mL) for 2 h. The resin was then washed with DMF (3×40 mL), and a solution of compound 411 (2.55 g, 6.000 mmol), HOBt (1.22 g, 9.000 mmol) and DIC (1.14 g, 9.000 mmol) in DMF (40 mL) was added. The reaction mixture was run for 2 h and the resin was washed with DMF (3×40 mL) to afford 414.

### Example 268: ((S)-7-((S)-7-(1-(9H-fluoren-9-yl)-3-oxo-2,7,10-trioxa-4-azatridecan-13-amido)-2,2-dimethyl-4,8-dioxo-3,12,15-trioxa-9-azaoctadecan-18-amido)-2,2-dimethyl-4,8-dioxo-3,12,15-trioxa-9-azaoctadecan-18-oyl)-L-valine-CTC resin (415)

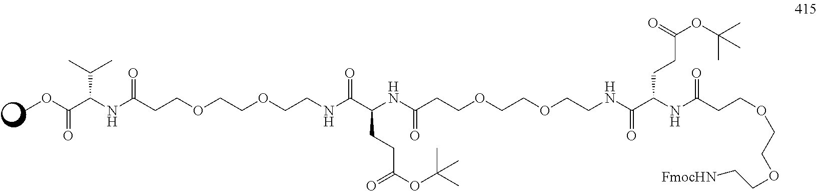

A solid phase peptide synthesis vessel (Chemgalss, 100-mL) was charged with 414. Fmoc deprotection was performed using piperidine (2.04 g, 24.000 mmol) in DMF (40 mL) for 2 h. The resin was then washed with DMF (3×40 mL), and a solution of compound 409 (2.55 g, 6.000 mmol), HOBt (1.22 g, 9.000 mmol) and DIC (1.14 g, 9.000 mmol) in DMF (40 mL) was added. The reaction mixture was run for 2 h and the resin was washed with DMF (3×40 mL) to afford 415.

### Example 269: ((S)-7-((S)-7-(3-(2-(2-(4-(2,5-dioxo-2,5-dihydro-1H-pyrrol-1-yl)butanamido) ethoxy)ethoxy)propanamido)-2,2-dimethyl-4,8-dioxo-3,12,15-trioxa-9-azaoctadecan-18-amido)-2,2-dimethyl-4,8-dioxo-3,12,15-trioxa-9-azaoctadecan-18-oyl)-L-valine-CTC resin (417)

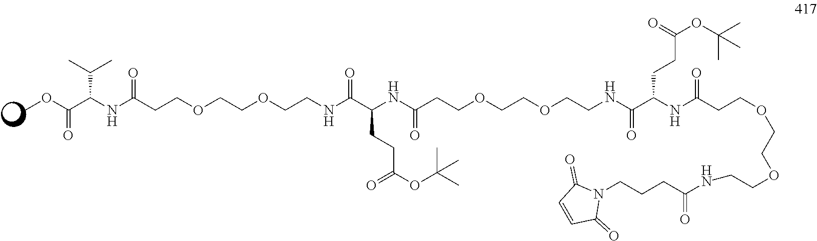 [SMILES: COC(=O)[C@@H](NC(=O)CCOCCOCCNC(=O)[C@H](CCC(=O)OC(C)(C)C)NC(=O)CCOCCOCCNC(=O)[C@H](CCC(=O)OC(C)(C)C)NC(=O)CCOCCOCCNC(=O)CCCN1C(=O)C=CC1=O)C(C)C]

A solid phase peptide synthesis vessel (Chemgalss, 100-mL) was charged with 415. Fmoc deprotection was performed using piperidine (2.04 g, 24.000 mmol) in DMF (40 mL) for 2 h. The resin was then washed with DMF (3×40 mL), and a solution of compound 416 (1.1 g, 6.000 mmol), HOBt (1.22 g, 9.000 mmol) and DIC (1.14 g, 9.000 mmol) in DMF (40 mL) was added. The reaction mixture was run for 2 h and the resin was washed with DMF (3×40 mL) to afford 417.

### Example 270: ((S)-7-((S)-7-(3-(2-(2-(4-(2,5-dioxo-2,5-dihydro-1H-pyrrol-1-yl)butanamido)ethoxy)ethoxy)propanamido)-2,2-dimethyl-4,8-dioxo-3,12,15-trioxa-9-azaoctadecan-18-amido)-2,2-dimethyl-4,8-dioxo-3,12,15-trioxa-9-azaoctadecan-18-oyl)-L-valine-CTC resin (419)

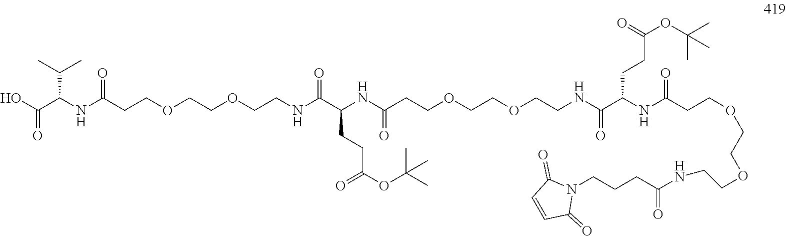 [SMILES: CC(C)[C@H](NC(=O)CCOCCOCCNC(=O)[C@H](CCC(=O)OC(C)(C)C)NC(=O)CCOCCOCCNC(=O)[C@H](CCC(=O)OC(C)(C)C)NC(=O)CCOCCOCCNC(=O)CCCN1C(=O)C=CC1=O)C(=O)O]

A solid phase peptide synthesis vessel (Chemgalss, 100-mL) were charged with 417, compound 418 (17 mL) and DCM (40 mL). The reaction mixture was run for 0.5 h and the resin was washed with DCM (3×40 mL), and the wash was concentrated in vacuo to afford 419 (3.47 g, 51% yield) as a colorless oil. MS ESI (m/z): calcd for C₅₂H₈₇N₇O₂₀ [M+H]⁺: 1130.61; found 1130.25.

### Example 271: 3,3′-azanediyldipropionic acid (422)

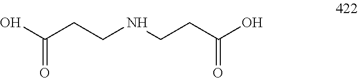 [SMILES: O=C(O)CCNCCC(=O)O]

To a solution of compound 421 (4.86 g, 18.601 mmol) and in DCM (50 mL) was added TFA (20 mL). The mixture was stirred at RT for 1 h and concentrated in vacuo to afford 422 (3 g, crude) as a colorless oil. MS ESI (m/z): calcd for C₆H₁NO₄ [M+H]⁺: 162.08; found 161.88.

### Example 272: 3,3′-(((benzyloxy)carbonyl) azanediyl) dipropionic acid (423)

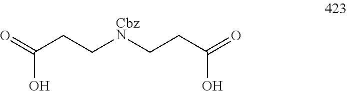 [SMILES: O=C(O)CCN(CCC(=O)O)C(=O)OCc1ccccc1]

To a solution of compound 422 (3.00 g, 18.615 mmol) in THF (30 mL) was added a solution of LiOH (3.12 g, 74.460 mmol) in H₂O (74 mL), and then a solution of Cbz-C₁ (4.89 g, 28.667 mmol) in THF (30 mL) was added dropwise at 0° C. The mixture was stirred at 0° C. for 30 min and concentrated in vacuo. The residue was acidified with HCl (2 mol/L) to pH=1, and then extracted with DCM (30 mL×2). The aqueous layer was saturated with solid NaCl, and extracted with DCM/MeOH (10/1) (40 mL×4). The combined organic layers were dried over anhydrous Na₂SO₄ and concentrated in vacuo to afford 423 (2.98 g, 54% yield) as a white solid. MS ESI (m/z): calcd for C₁₄H₁₇NO₆ [M+H]⁺: 296.11; found 295.87.

### Example 273: benzyl (1-((2R,4R,5R)-4-(((benzyloxy)carbonyl)oxy)-3,3-difluoro-5-((S)-6-methyl-5,8-dioxo-10-phenyl-2,9-dioxa-4,7-diazadecyl) tetrahydrofuran-2-yl)-2-oxo-1,2-dihydropyrimidin-4-yl)carbamate (426)

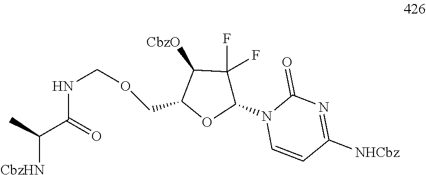

To a solution of compound 424 (3.00 g, 5.645 mmol) and compound 425 (3.32 g, 11.289 mmol) in THF (30 mL) was added PPTS (0.57 g, 2.258 mmol). The mixture was stirred at RT for 4 h and concentrated in vacuo. The residue was purified by prep-HPLC (eluted with CH₃CN/H₂O (containing 0.05% formic acid)=61/39) to afford 426 (2.02 g, 47% yield) as a white solid. MS ESI (m/z): calcd for C₃₇H₃₇F₂N₅O₁₁ [M+H]⁺: 766.25; found 765.86.

### Example 274: (S)-2-amino-N-((((2R,3R,5R)-5-(4-amino-2-oxopyrimidin-1 (2H)-yl)-4,4-difluoro-3-hydroxytetrahydrofuran-2-yl)methoxy)methyl)propanamide (427)

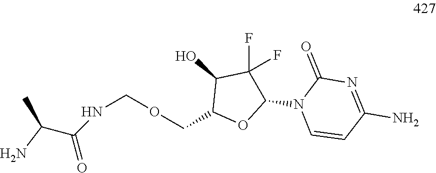

To a solution of compound 426 (2.02 g, 2.638 mmol) in MeOH (20 mL) were added Pd/C (200 mg, 10 wt %) and NH₃/MeOH (0.5 mL, 7 mol/L). The mixture was stirred at RT for 5 h under H₂ and then filtered over Celite, and the filtrate was concentrated in vacuo to afford 427 (0.96 g, crude) as a white solid. MS ESI (m/z): calcd for C₁₃H₁₉F₂N₅O₅ [M+H]⁺: 364.14; found 363.85.

### Example 275: benzyl ((S)-1-(((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl)amino)-3-methyl-1-oxobutan-2-yl)carbamate (429)

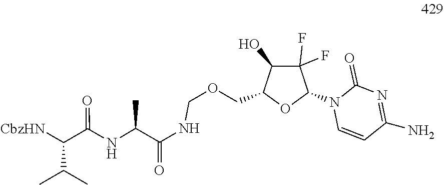 [SMILES: CC(C)[C@H](NC(=O)OCc1ccccc1)C(=O)N[C@@H](C)C(=O)NCOC[C@H]1O[C@@H](n2ccc(N)nc2=O)C(F)(F)[C@@H]1O]

To a solution of compound 427 (0.96 g, 2.642 mmol) and compound 428 (0.66 g, 2.642 mmol) in DMF (5 mL) were added HATU (1.11 g, 2.907 mmol) and DIPEA (0.68 g, 5.285 mmol). The mixture was stirred at RT for 10 min and concentrated in vacuo. The residue was purified by silica gel column chromatography (eluted with DCM/MeOH=10/1˜7/1) to afford 429 (1.46 g, 93% yield) as a colorless oil. MS ESI (m/z): calcd for C₂₆H₃₄F₂N₆O₈ [M+H]⁺: 597.25; found 596.79.

### Example 276: (S)-2-amino-N-((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl)-3-methylbutanamide (430)

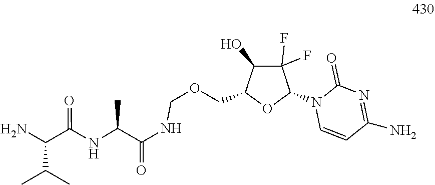 [SMILES: CC(C)[C@H](N)C(=O)N[C@@H](C)C(=O)NCOC[C@H]1O[C@@H](n2ccc(N)nc2=O)C(F)(F)[C@@H]1O]

To a solution of compound 429 (1.46 g, 2.447 mmol) in MeOH (15 mL) were added Pd/C (150 mg, 10 wt %) and NH₃/MeOH (0.5 mL, 7 mol/L). The mixture was stirred at RT for 4 h under H₂ and then filtered over Celite. The filtrate was concentrated in vacuo to afford 430 (1.13 g, crude) as a white solid. MS ESI (m/z): calcd for C₁₈H₂₈F₂N₆O₆ [M+H]⁺: 463.21; found 462.94.

### Example 277: benzyl bis((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2-oxa-4,7,10-triazatridecan-13-yl)carbamate (431)

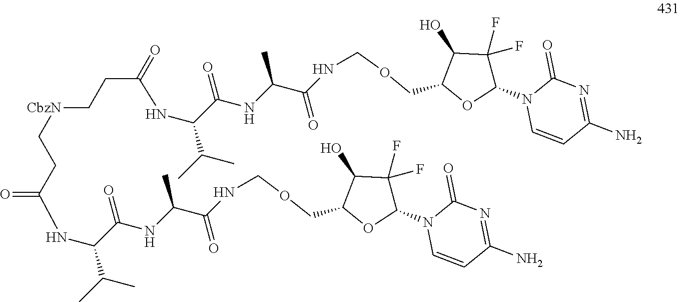 [SMILES: CC(C)[C@H](NC(=O)CCN(CCC(=O)N[C@H](C(=O)N[C@@H](C)C(=O)NCOC[C@H]1O[C@@H](n2ccc(N)nc2=O)C(F)(F)[C@@H]1O)C(C)C)C(=O)OCc1ccccc1)C(=O)N[C@@H](C)C(=O)NCOC[C@H]1O[C@@H](n2ccc(N)nc2=O)C(F)(F)[C@@H]1O]

To a solution of compound 430 (100 mg, 0.339 mmol) and compound 423 (329 mg, 0.711 mmol) in DMF (5 mL) were added HATU (283 mg, 0.745 mmol) and DIPEA (175 mg, 1.355 mmol). The mixture was stirred at RT for 10 min and concentrated in vacuo. The residue was purified by purified by prep-HPLC (eluted with CH₃CN/H₂O (containing 0.05% formic acid)=28/72) to afford 431 (180 mg, 45% yield) as a white solid. MS ESI (m/z): calcd for C₅₀H₆₉F₄N₁₃O₁₆ [M+H]⁺: 1184.50; found 1183.69.

### Example 278: (2S,2'S)-2,2′-((3,3′-azanediylbis(propanoyl))bis(azanediyl))bis(N-((S)-1-(((((2R,3R,5R)-5-(4-amino-2-oxopyrimidin-1 (2H)-yl)-4,4-difluoro-3-hydroxytetrahydrofuran-2-yl)methoxy)methyl)amino)-1-oxopropan-2-yl)-3-methylbutanamide) (432)

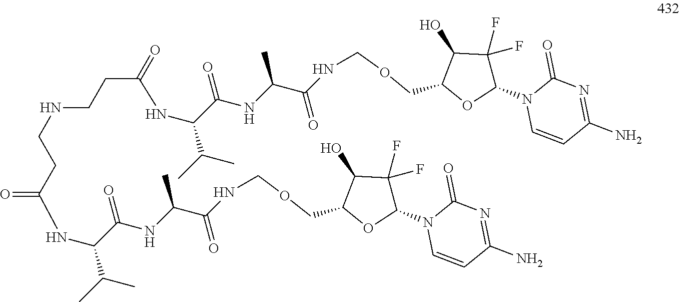 [SMILES: CC(C)[C@H](NC(=O)CCNCCC(=O)N[C@H](C(=O)N[C@@H](C)C(=O)NCOC[C@H]1O[C@@H](n2ccc(N)nc2=O)C(F)(F)[C@@H]1O)C(C)C)C(=O)N[C@@H](C)C(=O)NCOC[C@H]1O[C@@H](n2ccc(N)nc2=O)C(F)(F)[C@@H]1O]

To a solution of compound 431 (180 mg, 0.852 mmol) in MeOH (10 mL) were added Pd/C (18 mg, 10 wt %) and NH₃/MeOH (0.5 mL, 7 mol/L). The mixture was stirred at 50° C. for 2 h under H₂ and then filtered over Celite. The filtrate was concentrated in vacuo to afford 432 (159 mg, crude) as a white solid. MS ESI (m/z): calcd for C₄₂H₆₃F₄N₁₃O₁₄ [M+H]⁺: 1050.47; found 1049.82.

### Example 279: di-tert-butyl (4S,17S)-4-(((2S,5S)-1-((3-((2,5,8,11,14,17,20,23-octaoxapentacosan-25-yl)carbamoyl)-4-(hydroxymethyl)phenyl)amino)-5-isopropyl-2-methyl-1,4,7-trioxo-10,13-dioxa-3,6-diazapentadecan-15-yl)carbamoyl)-17-(3-(2-(2-(4-(2,5-dioxo-2,5-dihydro-1H-pyrrol-1-yl)butanamido)ethoxy)ethoxy)propanamido)-6,16-dioxo-9,12-dioxa-5,15-diazaicosanedioate (434)

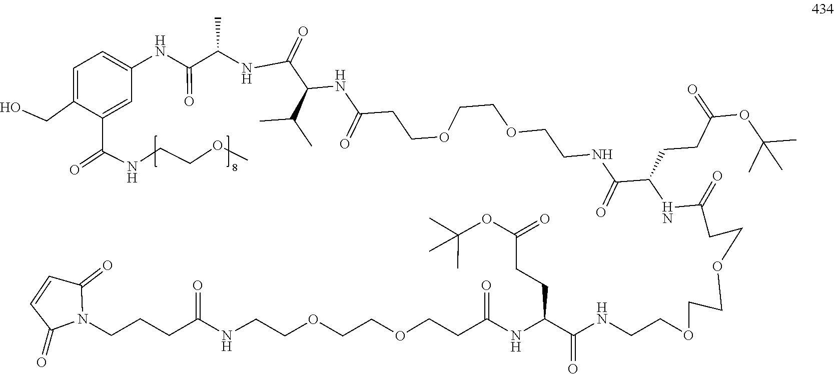

To a solution of compound 419 (3.47 g, 3.070 mmol) in DMF (20 mL) was added HATU (1.28 g, 3.377 mmol). The mixture was stirred at RT for 10 min, and compound 433 (1.85 g, 3.070 mmol) and DIPEA (1.19 g, 9.210 mmol) were added to the reaction. The mixture was stirred at RT for 10 min and diluted with water (40 mL). After extraction with DCM (40 mL×3), the combined organic layers were washed with H₂O (40 mL×3), dried over anhydrous Na₂SO₄ and concentrated in vacuo. The residue was purified by silica gel column chromatography (eluted with DCM/MeOH=15/1˜10/1) to afford 434 (2.91 g, 55% yield) as a yellow oil. MS ESI (m/z): calcd for C₈₀H₁₃₄N₁₀O₃₀ [M+H]⁺: 1715.94; found 1716.74.

### Example 280: Synthesis of di-tert-butyl (4S,17S)-4-(((2S,5S)-1-((3-((2,5,8,11,14,17,20,23-octaoxapentacosan-25-yl)carbamoyl)-4-((((4-nitrophenoxy)carbonyl)oxy)methyl)phenyl)amino)-5-isopropyl-2-methyl-1,4,7-trioxo-10,13-dioxa-3,6-diazapentadecan-15-yl)carbamoyl)-17-(3-(2-(2-(4-(2,5-dioxo-2,5-dihydro-1H-pyrrol-1-yl)butanamido)ethoxy)ethoxy)propanamido)-6,16-dioxo-9,12-dioxa-5,15-diazaicosanedioate (435)

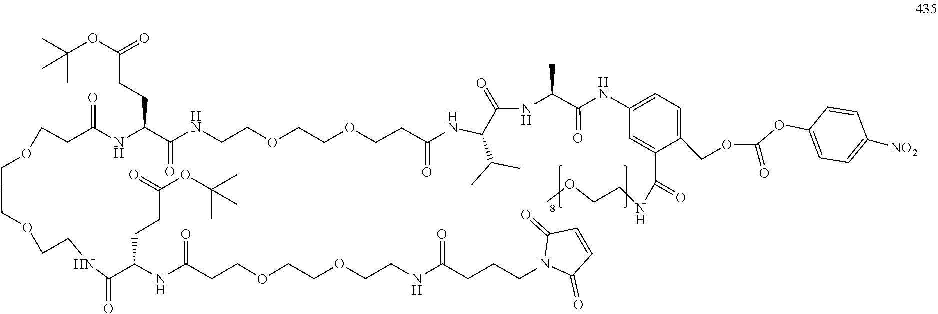

To a solution of compound 434 (2.91 g, 1.696 mmol) and 4-nitrophenyl carbonochloridate (0.68 g, 3.392 mmol) in DCM (20 mL) was added pyridine (0.27 g, 3.392 mmol). The mixture was stirred at RT for 5 min and concentrated in vacuo. The residue was purified by prep-HPLC (eluted with CH₃CN/H₂O=50/50) to afford 435 (2.32 g, 73% yield) as a yellow solid. MS ESI (m/z): calcd for C₈₇H₁₃₇N₁₁O₃₄ [M+H]⁺: 1880.94; found 1880.46.

### Example 281: (4S,17S)-4-(((2S,5S)-1-((3-((2,5,8,11,14,17,20,23-octaoxapentacosan-25-yl) carbamoyl)-4-((((4-nitrophenoxy)carbonyl)oxy)methyl)phenyl)amino)-5-isopropyl-2-methyl-1,4,7-trioxo-10,13-dioxa-3,6-diazapentadecan-15-yl)carbamoyl)-17-(3-(2-(2-(4-(2,5-dioxo-2,5-dihydro-1H-pyrrol-1-yl)butanamido)ethoxy)ethoxy)propanamido)-6,16-dioxo-9,12-dioxa-5,15-diazaicosanedioic acid (436)

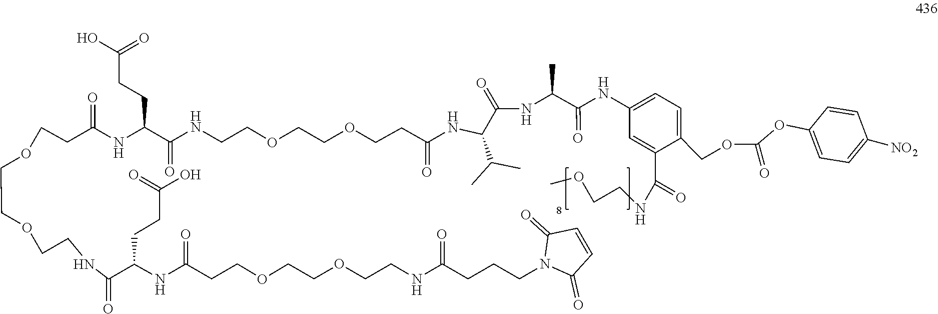

To a solution of compound 435 (2.32 g, 1.233 mmol) in DCM (30 mL) was added TFA (20 mL). The mixture was stirred at RT for 1 h. The reaction mixture was concentrated in vacuo to afford 436 (2.18 g, crude) as a colorless oil. MS ESI (m/z): calcd for C₇₉H₁₂₁N₁₁O₃₄ [M+H]⁺: 1768.82; found 1768.19.

### Example 282: di-tert-butyl (20S,33S)-20-(((2S,5S)-1-((3-((2,5,8,11,14,17,20,23-octaoxapentacosan-25-yl)carbamoyl)-4-((((4-nitrophenoxy)carbonyl)oxy)methyl)phenyl)amino)-5-isopropyl-2-methyl-1,4,7-trioxo-10,13-dioxa-3,6-diazapentadecan-15-yl)carbamoyl)-33-(3-(2-(2-(4-(2,5-dioxo-2,5-dihydro-1H-pyrrol-1-yl)butanamido)ethoxy)ethoxy)propanamido)-17,22,32,36-tetraoxo-4,7,10,13,25,28,40,43,46,49-decaoxa-16,21,31,37-tetraazadopentacontanedioate (437)

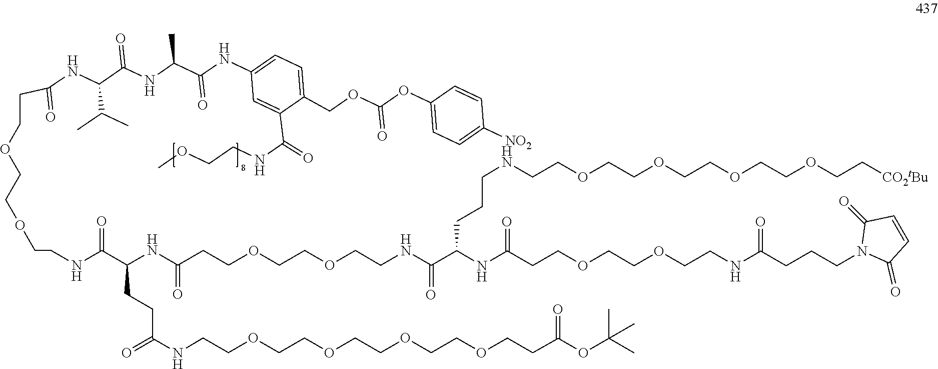

To a solution of compound 436 (2.18 g, 1.232 mmol) and compound 276 (0.83 g, 2.588 mmol) in DMF (20 mL) were added HATU (1.03 g, 2.711 mmol) and DIPEA (0.64 g, 4.93 mmol). The mixture was stirred at RT for 10 min and concentrated in vacuo. The residue was purified by prep-HPLC (eluted with CH₃CN/H₂O=51/49) to afford 437 (1.36 g, 46% yield) as a yellow oil. MS ESI (m/z): calcd for C₁₀₉H₁₇₉N₁₃O₄₄ [M+H]⁺: 2375.23; found 2374.64.

### Example 283: (20S,33S)-20-(((2S,5S)-1-((3-((2,5,8,11,14,17,20,23-octaoxapentacosan-25-yl) carbamoyl)-4-((((4-nitrophenoxy)carbonyl)oxy)methyl)phenyl)amino)-5-isopropyl-2-methyl-1,4,7-trioxo-10,13-dioxa-3,6-diazapentadecan-15-yl)carbamoyl)-33-(3-(2-(2-(4-(2,5-dioxo-2,5-dihydro-1H-pyrrol-1-yl)butanamido)ethoxy)ethoxy)propanamido)-17,22,32,36-tetraoxo-4,7,10,13,25,28,40,43,46,49-decaoxa-16,21,31,37-tetraazadopentacontanedioic acid (438)

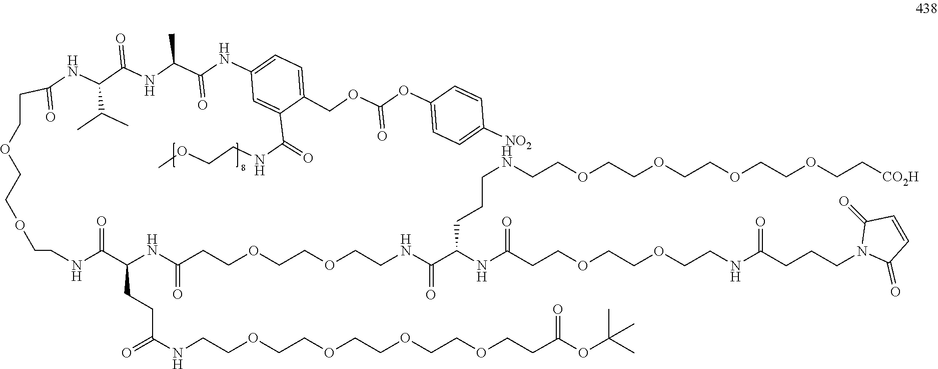

To a solution of compound 437 (180 mg, 0.076 mmol) in DCM (5 mL) was added TFA (2 mL). The mixture was stirred at RT for 1 h and then concentrated in vacuo to afford 438 (171 mg, crude) as a colorless oil. MS ESI (m/z): calcd for C₁₀₁H₁₆₃N₁₃O₄₄ [M+H]⁺: 2263.10; found 2263.10.

### Example 284: 2-((2,5,8,11,14,17,20,23-octaoxapentacosan-25-yl)carbamoyl)-4-((6S,9S,34S,47S,60S,63S)-1-((2R,3R,5R)-5-(4-amino-2-oxopyrimidin-1 (2H)-yl)-4,4-difluoro-3-hydroxytetrahydrofuran-2-yl)-47-((6S,9S)-1-((2R,3R,5R)-5-(4-amino-2-oxopyrimidin-1 (2H)-yl)-4,4-difluoro-3-hydroxytetrahydrofuran-2-yl)-14-((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2-oxa-4,7,10-triazatridecan-13-yl)-9-isopropyl-6-methyl-5,8,11,15,31-pentaoxo-2,18,21,24,27-pentaoxa-4,7,10,14,30-pentaazatritriacontan-33-yl)-14-((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2-oxa-4,7,10-triazatridecan-13-yl)-34-(3-(2-(2-(4-(2,5-dioxo-2,5-dihydro-1H-pyrrol-1-yl)butanamido)ethoxy)ethoxy)propanamido)-9,60-diisopropyl-6,63-dimethyl-5,8,11,15,31,35,45,48,58,61-decaoxo-2,18,21,24,27,39,42,52,55-nonaoxa-4,7,10,14,30,36,46,49,59,62-decaazatetrahexacontan-64-amido)benzyl (4-nitrophenyl) carbonate (439)

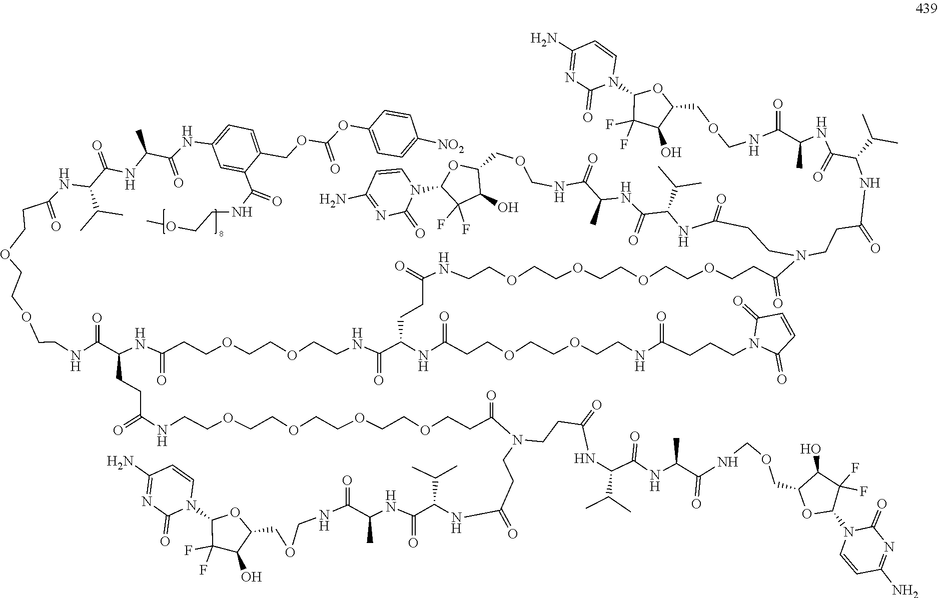

To a solution of compound 438 (171 mg, 0.076 mmol) and compound 432 (159 mg, 0.151 mmol) in DMF (5 mL) were added HATU (63 mg, 0.166 mmol) and DIPEA (39 mg, 0.302 mmol). The mixture was stirred at RT for 30 min, and used for the next step without work-up. MS ESI (m/z): calcd for C₁₈₅H₂₈₅F₈N₃₉O₇₀ [M+2H]₂₊: 2163.49; found 2163.93.

### Example 285: 2-((2,5,8,11,14,17,20,23-octaoxapentacosan-25-yl)carbamoyl)-4-((6S,9S,34S,47S,60S,63S)-1-((2R,3R,5R)-5-(4-amino-2-oxopyrimidin-1 (2H)-yl)-4,4-difluoro-3-hydroxytetrahydrofuran-2-yl)-47-((6S,9S)-1-((2R,3R,5R)-5-(4-amino-2-oxopyrimidin-1 (2H)-yl)-4,4-difluoro-3-hydroxytetrahydrofuran-2-yl)-14-((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2-oxa-4,7,10-triazatridecan-13-yl)-9-isopropyl-6-methyl-5,8,11,15,31-pentaoxo-2,18,21,24,27-pentaoxa-4,7,10,14,30-pentaazatritriacontan-33-yl)-14-((6S,9S)-1-((2R,3R,5R)-5-(4-amino-2-oxopyrimidin-1 (2H)-yl)-4,4-difluoro-3-hydroxytetrahydrofuran-2-yl)-9-isopropyl-6-methyl-5,8,11-trioxo-2-oxa-4,7,10-triazatridecan-13-yl)-34-(3-(2-(2-(4-(2,5-dioxo-2,5-dihydro-1H-pyrrol-1-yl)butanamido)ethoxy)ethoxy)propanamido)-9,60-diisopropyl-6,63-dimethyl-5,8,11,15,31,35,45,48,58,61-decaoxo-2,18,21,24,27,39,42,52,55-nonaoxa-4,7,10,14,30,36,46,49,59,62-decaazatetrahexacontan-64-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (440)

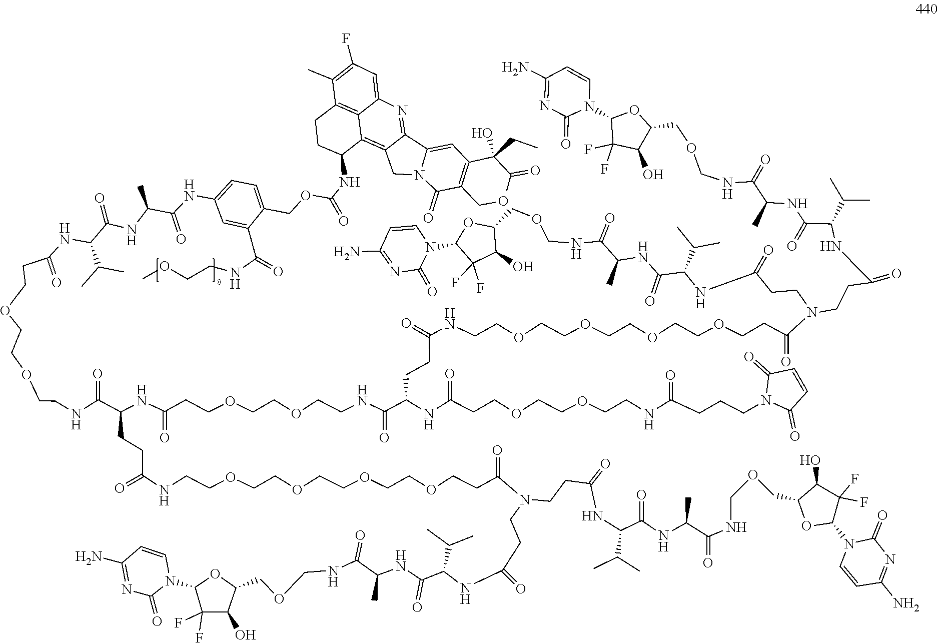

To a solution of compound 439 (326 mg, assuming 0.076 mmol) and Exatecan mesylate salt (40 mg, 0.076 mmol) in DMF (5 mL) were added HOBt (10 mg, 0.076 mmol) and DIPEA (29 mg, 0.228 mmol). The mixture was stirred at RT for 3 h and concentrated in vacuo. The residue was purified by purified by prep-HPLC (eluted with CH₃CN/H₂O (containing 0.05% formic acid)=32/68) to afford 440 (95 mg, 27% yield) as a white solid. MS ESI (m/z): calcd for C₂₀₃H₃₀₂F₉N₄₁O₇₁ [M+3H]³⁺: 1541.37; found 1541.88.

### Example 286: benzyl (1-((2R,4R,5R)-4-(((benzyloxy)carbonyl)oxy)-5-(5,8-dioxo-10-phenyl-2,9-dioxa-4,7-diazadecyl)-3,3-difluorotetrahydrofuran-2-yl)-2-oxo-1,2-dihydropyrimidin-4-yl)carbamate (442)

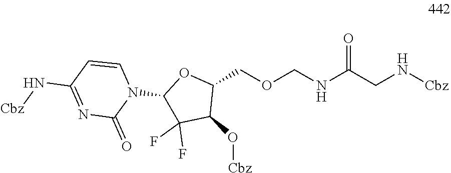 [SMILES: O=C(CNC(=O)OCc1ccccc1)NCOC[C@H]1O[C@@H](n2ccc(NC(=O)OCc3ccccc3)nc2=O)C(F)(F)[C@@H]1OC(=O)OCc1ccccc1]

To a solution of 294 (3.00 g, 5.645 mmol, 1.0 eq) and (2-(((benzyloxy)carbonyl)amino)-acetamido) methyl acetate (3.16 g, 11.289 mmol, 2.0 eq) in THF (10.0 mL) was added PPTS (0.57 g, 2.258 mmol, 0.4 eq). The reaction mixture was stirred at room temperature for 12 h and concentrated under reduced pressure to give a residue. The residue was purified by prep-HPLC to afford 442 (1.00 g, 23.6% yield) as a white solid. ESI MS m/z: calcd for C₃₆H₃₅F₂N₅O₁₁ [M+H]⁺ 752.23 found 752.67.

### Example 287:2-amino-N-((((2R,3R,5R)-5-(4-amino-2-oxopyrimidin-1 (2H)-yl)-4,4-difluoro-3-hydroxytetrahydrofuran-2-yl)methoxy)methyl) acetamide (443)

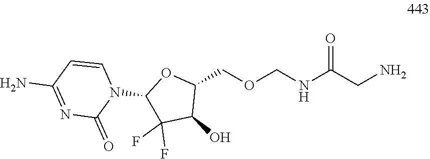 [SMILES: NCC(=O)NCOC[C@H]1O[C@@H](n2ccc(N)nc2=O)C(F)(F)[C@@H]1O]

To a solution of 442 (1.00 g, 1.330 mmol) in DMF (20 mL) at room temperature was added Pd/C (140.0 mg, 10 wt %). The resulting mixture stirred at room temperature under a H₂ balloon for 10 h before it was filtered through a pad of celite. The filtrate was concentrated under reduced pressure to afford 443 (460.0 mg, 100.0% yield) as brown oil. ESI MS m/z: [M+H]⁺ calcd for C₁₂H₁₇F₂N₅O₅ 350.12 found 350.20.

### Example 288. (9H-fluoren-9-yl)methyl ((S)-1-((2-(((((2R,3R,5R)-5-(4-amino-2-oxopyrimidin-1 (2H)-yl)-4,4-difluoro-3-hydroxytetrahydrofuran-2-yl)methoxy)methyl)amino)-2-oxoethyl)amino)-1-oxopropan-2-yl)carbamate (444)

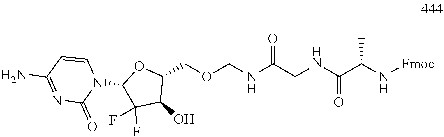

To a solution of 444 (697.0 mg, 1.995 mmol, 1.0 eq) and (((9H-fluoren-9-yl)methoxy)carbonyl)-L-alanine (621.3 mg, 1.995 mmol, 1.0 eq) and HATU (796.7 mg, 2.095 mmol, 1.05 eq) in DMF (10.0 mL) was added DIEA (386.9 mg, 2.993 mmol, 0.4 eq). The reaction mixture was stirred at room temperature for 1 h and concentrated under reduced pressure. The residue was purified by flash column chromatorgraphy to afford 444 (800.0 mg, 62.5% yield) as a white foam. ESI MS m/z: calcd for C₃₀H₃₂F₂N₆O₈ [M+H]⁺ 643.22 found 642.80.

### Example 289. (S)-2-amino-N-(2-(((((2R,3R,5R)-5-(4-amino-2-oxopyrimidin-1 (2H)-yl)-4,4-difluoro-3-hydroxytetrahydrofuran-2-yl)methoxy)methyl)amino)-2-oxoethyl)propanamide (445)

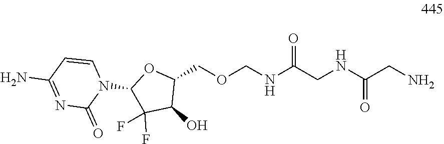 [SMILES: NCC(=O)NCC(=O)NCOC[C@H]1O[C@@H](n2ccc(N)nc2=O)C(F)(F)[C@@H]1O]

To a solution of 444 (800.0 mg, 1.245 mmol, 1.0 eq) in DMF (10 mL) was added piperidine (171 μL, 1.868 mmol, 1.5 eq). The mixture was stirred at room temperature for 1 h and concentrated under reduced pressure to afford 445 (520 mg, 100.0% yield) as a yellow solid. ESI MS m/z: [M+H]⁺ calcd for C₁₅H₂₂F₂N₆O₆ 421.16 found 420.79.

### Example 290: Synthesis of (9H-fluoren-9-yl)methyl ((9S,12S)-1-((2R,3R,5R)-5-(4-amino-2-oxopyrimidin-1 (2H)-yl)-4,4-difluoro-3-hydroxytetrahydrofuran-2-yl)-9,13-dimethyl-5,8,11-trioxo-2-oxa-4,7,10-triazatetradecan-12-yl)carbamate (446)

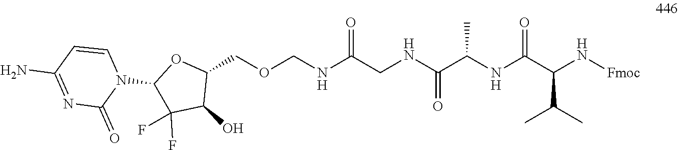

To a solution of 445 (520.0 mg, 1.237 mmol, 1.0 eq) and (((9H-fluoren-9-yl)methoxy)carbonyl)-L-valine (419.8 mg, 1.237 mmol, 1.0 eq) and HATU (493.9 mg, 1.299 mmol, 1.05 eq) in DMF (5.0 mL) was added DIEA (239.8 mg, 1.855 mmol, 1.5 eq). The reaction mixture was stirred at room temperature for 15 min and concentrated under reduced pressure. The residue was purified by flash column chromatorgraphy to afford 446 (581.0 mg, 63.2% yield) as a white foam. ESI MS m/z: calcd for C₃₅H₄₁F₂N₇O₉ [M+H] 742.29 found 741.84.

### Example 291. (S)-2-amino-N-((S)-1-((2-(((((2R,3R,5R)-5-(4-amino-2-oxopyrimidin-1 (2H)-yl)-4,4-difluoro-3-hydroxytetrahydrofuran-2-yl)methoxy)methyl)amino)-2-oxoethyl)amino)-1-oxopropan-2-yl)-3-methylbutanamide (447)

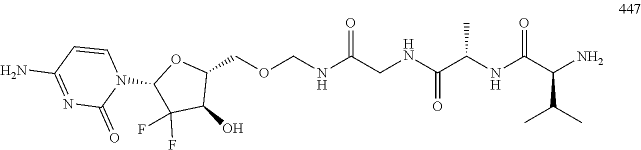 [SMILES: CC(C)[C@H](N)C(=O)N[C@@H](C)C(=O)NCC(=O)NCOC[C@H]1O[C@@H](n2ccc(N)nc2=O)C(F)(F)[C@@H]1O]

To a solution of 446 (581.0 mg, 0.783 mmol, 1.0 eq) in DMF (5 mL) was added piperidine (108 μL, 1.175 mmol, 1.5 eq). The mixture was stirred at room temperature for 1 h and concentrated under reduced pressure. The residue was purified by prep-HPLC to afford 447 (162.0 mg, 39.5% yield) as a colorless oil. ESI MS m/z: [M+H]⁺ calcd for C₂₀H₃₁F₂N₇O₇ 520.23 found 519.90.

### Example 292. 2-((2,5,8, 11,14,17,20,23-octaoxapentacosan-25-yl)carbamoyl)-4-((37S,45S,48S,51S)-45-((9S,12S)-1-((2R,3R,5R)-5-(4-amino-2-oxopyrimidin-1 (2H)-yl)-4,4-difluoro-3-hydroxy-tetrahydrofuran-2-yl)-12-isopropyl-9-methyl-5,8,11,14-tetraoxo-2-oxa-4,7,10,13-tetraazahexadecan-16-yl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (448)

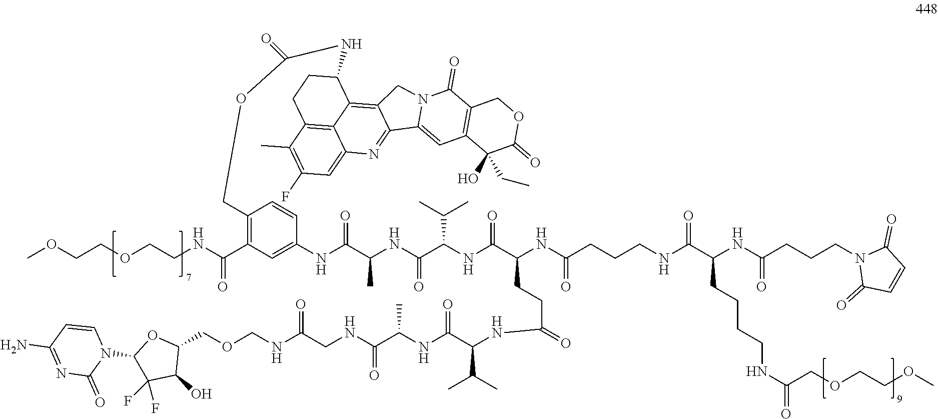

To a solution of 19 (370.8 mg, 0.173 mmol, 1.0 eq) in DMF (3 mL) was added HATU (69.2 mg, 0.182 mmol, 1.05 eq) at room temperature. The mixture was stirred at room temperature for 10 min. Then 447 (90.0 mg, 0.173 mmol, 1.0 eq) and DIEA (33.6 mg, 0.260 mmol, 1.5 eq) were added. The reaction mixture was stirred at room temperature for 0.5 h and quenched by formic acid (0.5 mL), then purified by prep-HPLC to afford 448 (271.0 mg, 58.9% yield) as a white solid. ESI MS m/z: [M+2H]⁺calcd for C₁₂₂H₁₈₂F₃N₁₉O_{422+1321.13} found 1321.10.

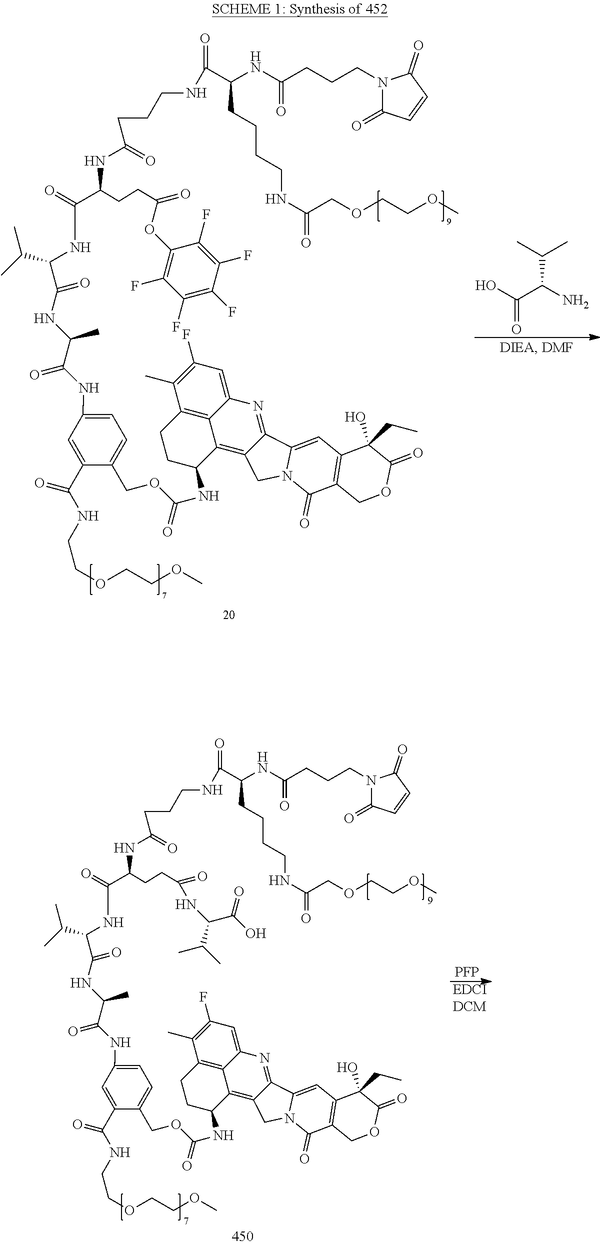

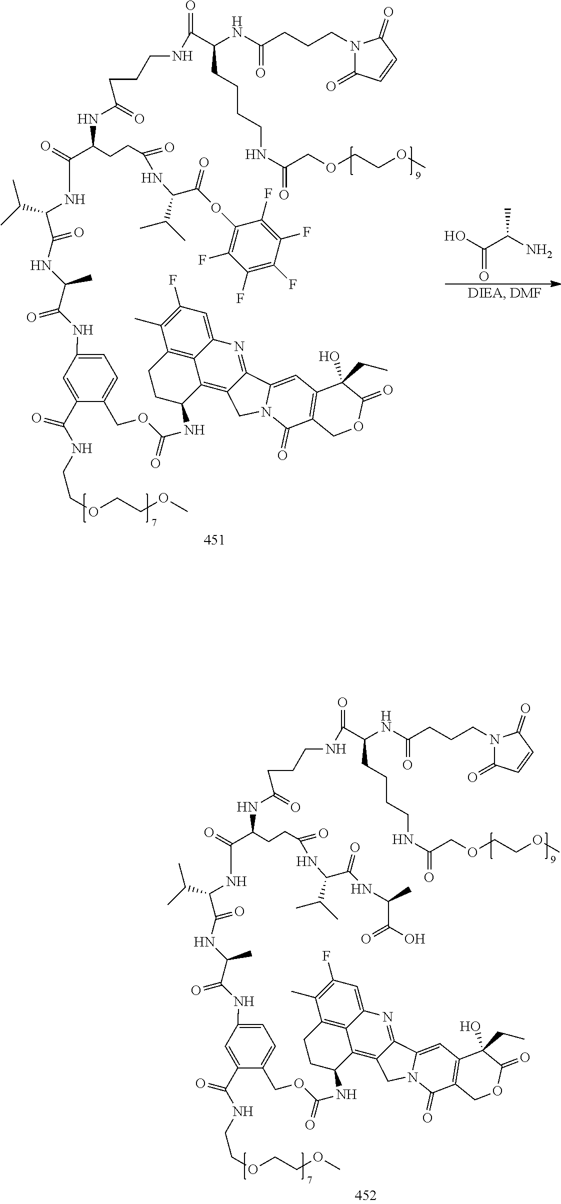

### Example 300: ((37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl) carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-oyl)-L-valine (450)

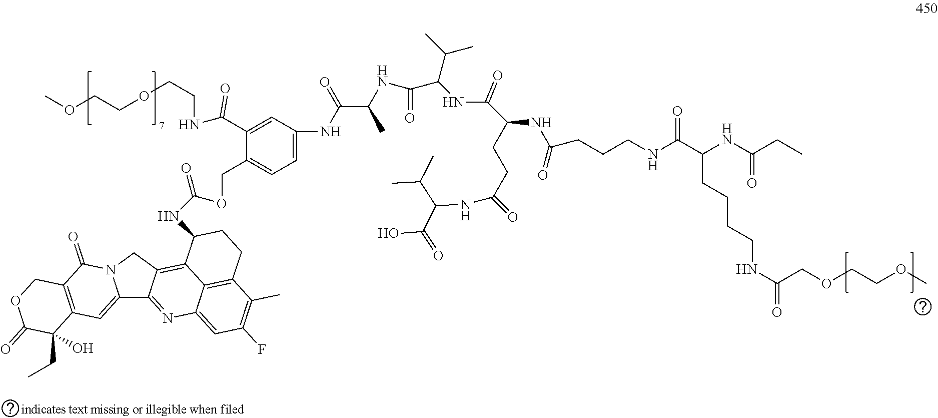

To a solution of compound 20 (1.00 eq) in DMF (1000% v/w) at 0° C. were added L-valine (1.20 eq) and DIEA (2.00 eq). The mixture was stirred at room temperature for 30 minutes and then purified by prep-HPLC, followed by lyophilization to afford 450. MS ESI (m/z): calcd for C₁₀₇H₁₆₀FN₁₃O₃₇ [M+H]⁺: 2239.10; found 2239.48.

### Example 301: perfluorophenyl ((37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-oyl)-L-valinate (451)

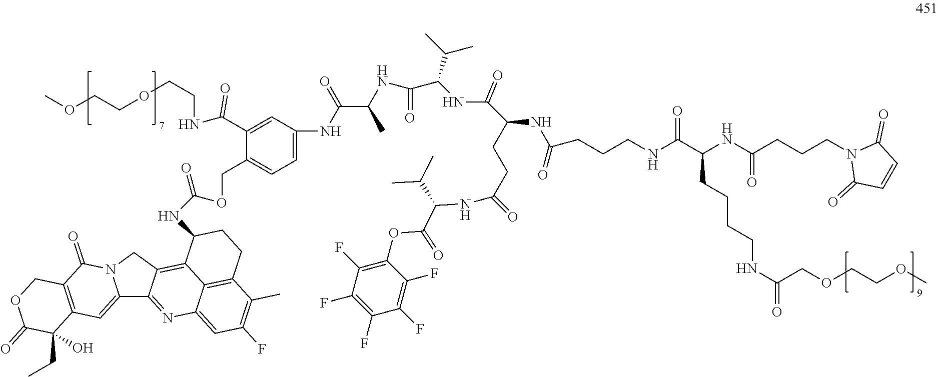

To a solution of compound 450 (1.00 eq) in DCM (1000% v/w) at 0° C. were added PFP-OH (1.20 eq) and EDCI (1.30 eq). The resulting mixture was stirred at 0° C. for 20 min, warmed to RT and stirred for 2 h before it was quenched with H₂O. The layers were separated, and the aqueous layer was extracted with CH₂Cl₂. The combined organic layers were dried over Na₂SO₄ and concentrated in vacuo to afforded 451. MS ESI (m/z): calcd for C₁₁₃H₁₅₉F₆N₁₃O₃₇ [M+H]⁺: 2405.09; found 2405.18.

### Example 302: ((37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl) carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8, 11,14,17,20,23,26,29-decaoxa-32,39,44-triazaoctatetracontan-48-oyl)-L-valyl-L-alanine (452)

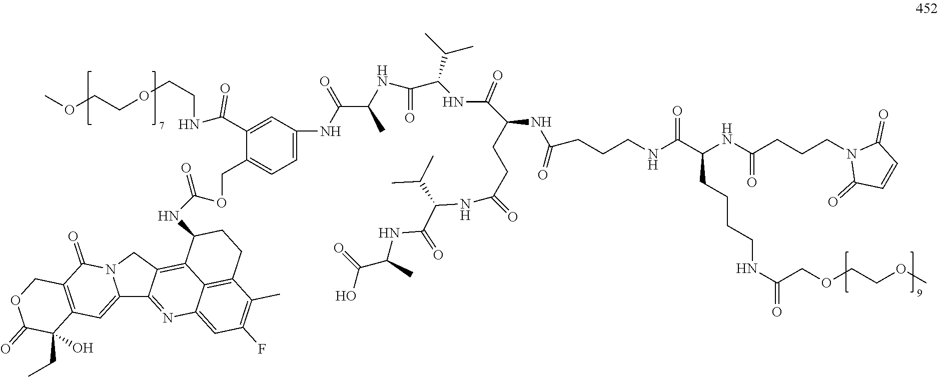

To a solution of compound 451 (1.00 eq) in DMF (1000% v/w) at 0° C. were added L-valine (1.20 eq) and DIEA (2.00 eq). The mixture was stirred at 0° C. for 20 min, warmed to RT and stirred at room temperature for 30 min before it was purified by prep-HPLC, followed by lyophilization to afforded 452. MS ESI (m/z): calcd for C₁₁₀H₁₆₅FN₁₄O₃₈ [M+H]⁺: 2310.14; found 2310.56.

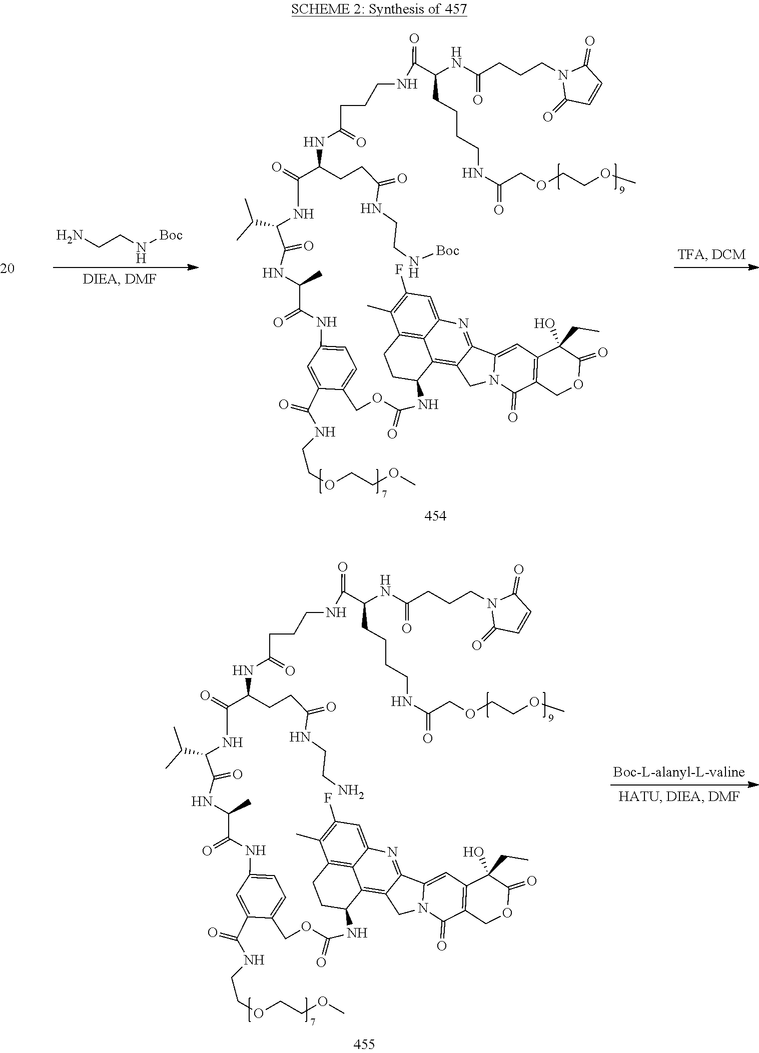

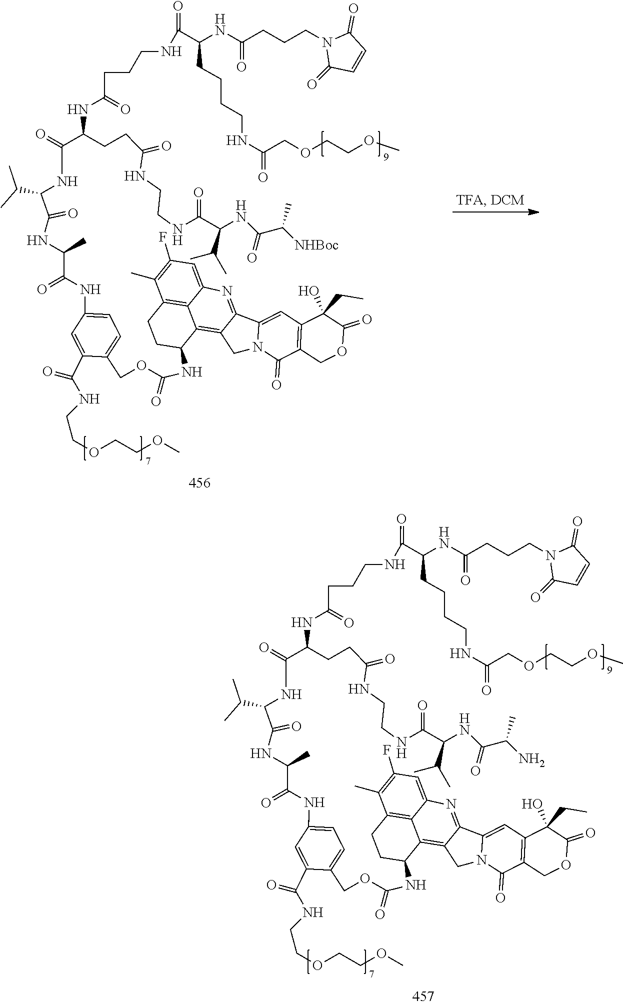

### Example 303: 2-((2,5,8,11,14,17,20,23-octaoxapentacosan-25-yl)carbamoyl)-4-((37S,45S,48S,51S)-45-(3-((2-((tert-butoxycarbonyl)amino) ethyl)amino)-3-oxopropyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (454)

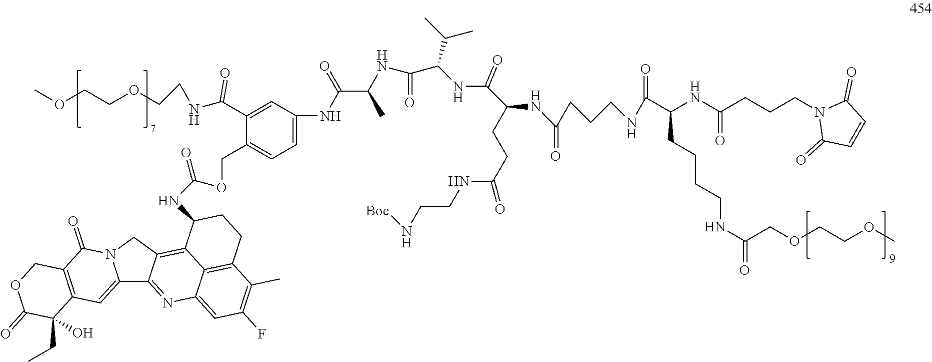

To a solution of compound 20 (1.00 eq) in DMF (1000% v/w) at 0° C. were added tert-butyl (2-aminoethyl)carbamate (1.20 eq) and DIEA (2.00 eq). The mixture was stirred at room temperature for 30 minutes and then purified by prep-HPLC, followed by lyophilization to afford 454. MS ESI (m/z): calcd for C₁₀₉H₁₆₅FN₁₄O₃₇ [M+H]⁺: 2282.14; found 2282.16.

### Example 304: 2-((2,5,8,11,14,17,20,23-octaoxapentacosan-25-yl)carbamoyl)-4-((37S,45S,48S,51S)-45-(3-((2-aminoethyl)amino)-3-oxopropyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (455)

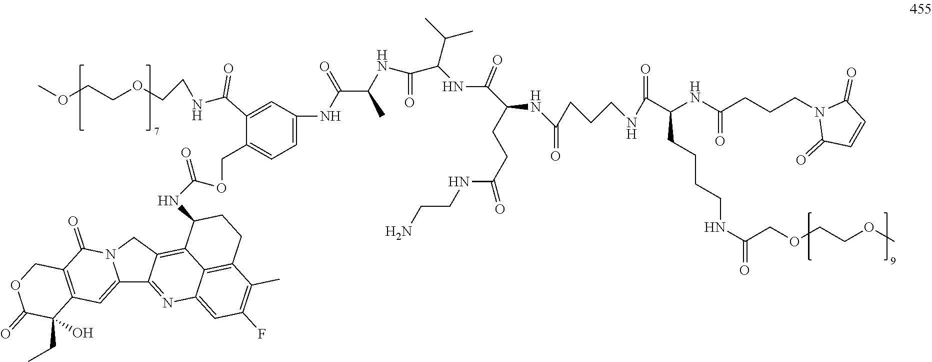

To a solution of compound 454 (1.00 eq) in DCM (500% v/w) was added TFA (500% vol/w). The mixture was stirred at RT for 1 h, and then concentrated in vacuo to afford 455 (2.18 g, crude) as a colorless oil. MS ESI (m/z): calcd for C₁₀₄H₁₅₇FN₁₄O₃₅ [M+H]⁺: 2182.09; found 2182.56.

### Example 305: 2-((2,5,8,11,14,17,20,23-octaoxapentacosan-25-yl)carbamoyl)-4-((37S,45S,48S,51S)-37-(4-(2,5-dioxo-2,5-dihydro-1H-pyrrol-1-yl)butanamido)-48-isopropyl-45-((6S,9S)-9-isopropyl-2,2,6-trimethyl-4,7,10,15-tetraoxo-3-oxa-5,8,11,14-tetraazaheptadecan-17-yl)-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (456)

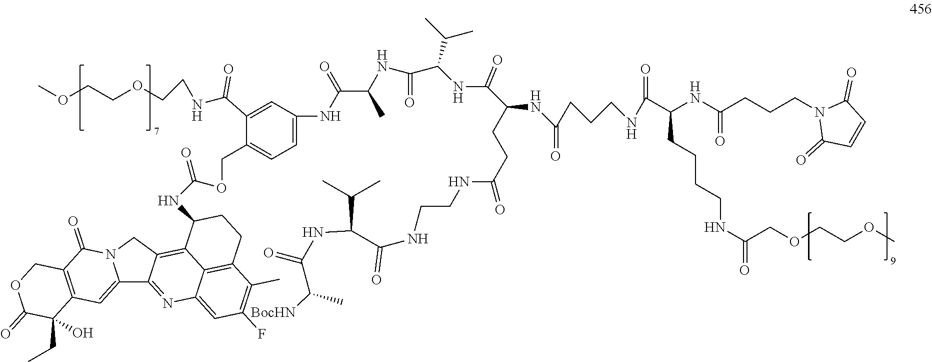

To a solution of Boc-L-alanyl-L-valine (1.50 eq) in DMF (1000% v/w) was added HATU (1.50 eq). After stirring for 10 min, compound 455 (1.00 eq) and DIPEA (2.00 eq) were added. The mixture was stirred at RT for 10 min, and then concentrated in vacuo. The residue was purified by prep-HPLC to afford 456. MS ESI (m/z): calcd for C₁₁₇H₁₇₉FN₁₆O₃₉ [M+H]⁺: 2452.25; found 2452.25.

### Example 306: 2-((2,5,8,11,14,17,20,23-octaoxapentacosan-25-yl)carbamoyl)-4-((37S,45S,48S,51S)-45-(3-((2-((S)-2-((S)-2-aminopropanamido)-3-methylbutanamido)ethyl)amino)-3-oxopropyl)-37-(4-(2,5-dioxo-2,5-dihydro-1H-pyrrol-1-yl)butanamido)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamate (457)

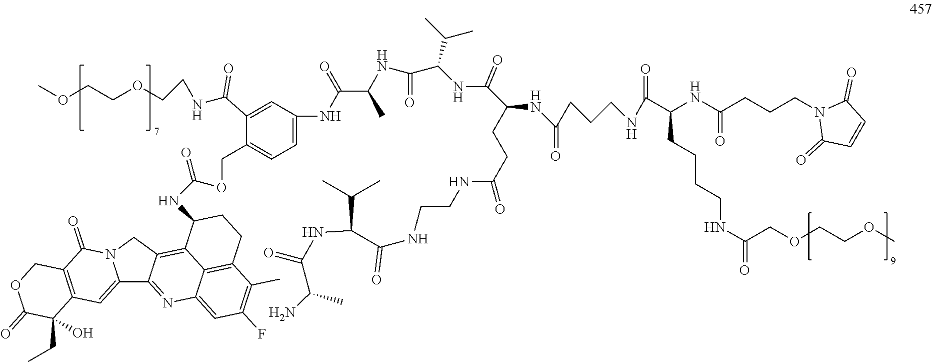

To a solution of compound 456 (1.00 eq) in DCM (500% v/w) was added TFA (500% vol/w). The mixture was stirred at RT for 1 h, and then concentrated in vacuo to afford 457 (2.18 g, crude) as a colorless oil. MS ESI (m/z): calcd for C₁₁₂H₁₇₁FN₁₆O₃₇ [M+H]⁺: 2352.20; found 2352.8.

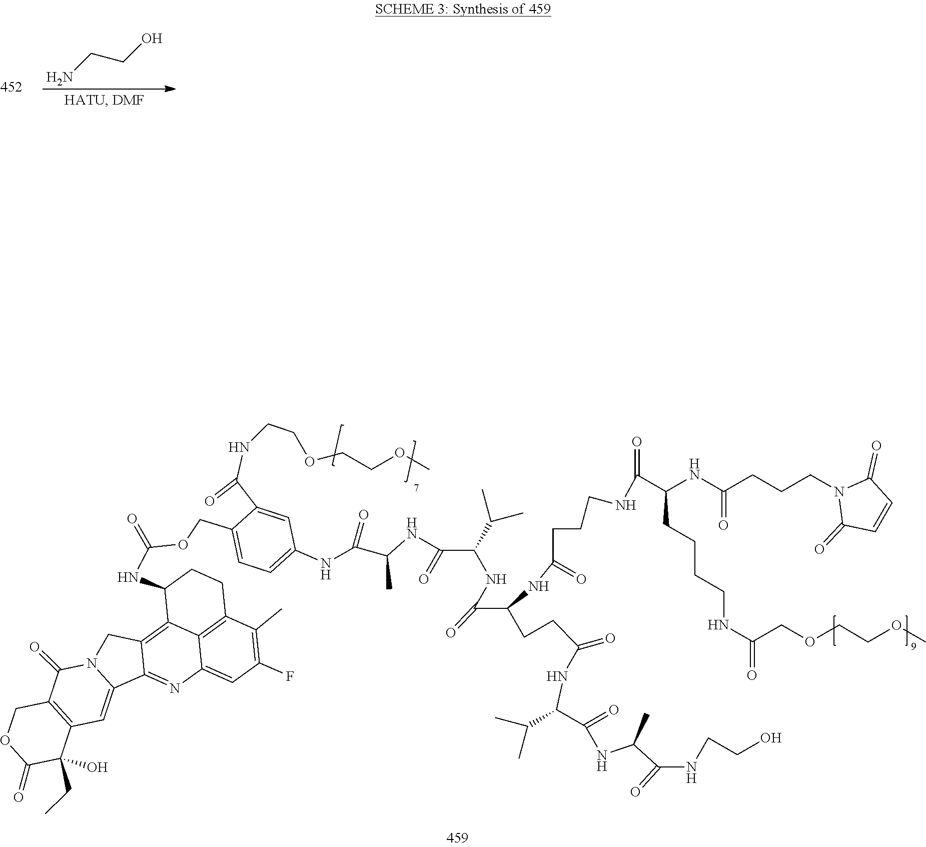

### Example 307: 2-((2,5,6,9,10,13,14, 17,18,21,22,25,26,29-tetradecaoxahentriacontan-31-yl)carbamoyl)-4-((37S,45S,48S,51S)-37-(4-(2,5-dioxo-2,5-dihydro-1H-pyrrol-1-yl)butanamido)-45-(3-((2-hydroxyethyl)amino)-3-oxopropyl)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamate (459)

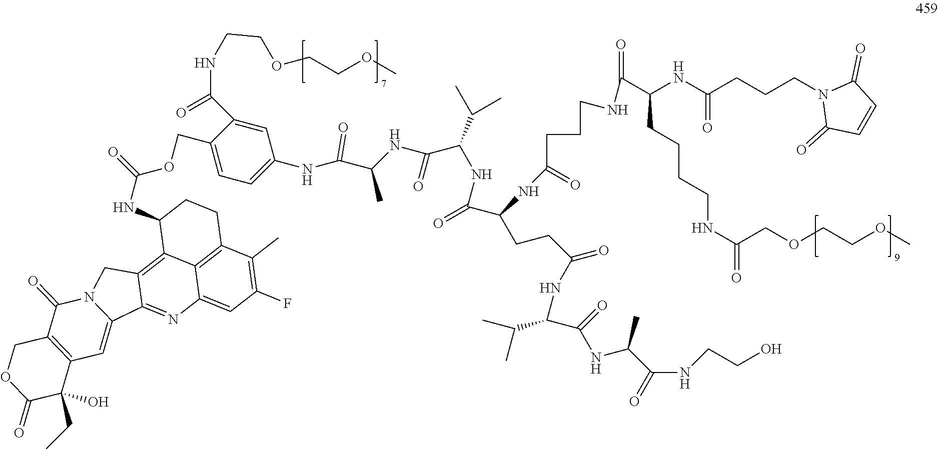

To a solution of compound 452 (1.00 eq), 2-aminoethan-1-ol (1.50 eq) in DMF (1000% v/w) were added HATU (1.50 eq) and DIPEA (2.00 eq). The mixture was stirred at RT for 1 h, and then concentrated in vacuo. The residue was purified by prep-HPLC to afford 459. MS ESI (m/z): calcd for C₁₁₂H₁₇₀FN₁₅O₃₈ [M+H]⁺: 2353.18; found 2352.94.

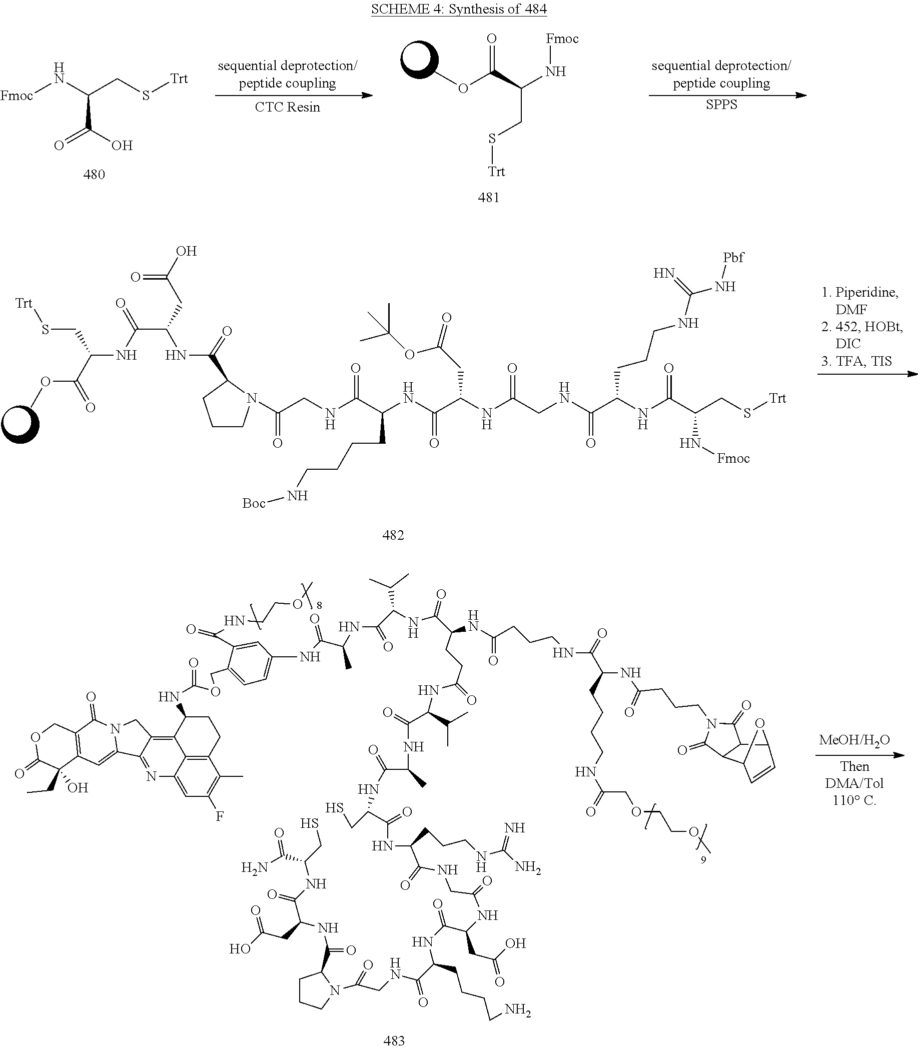

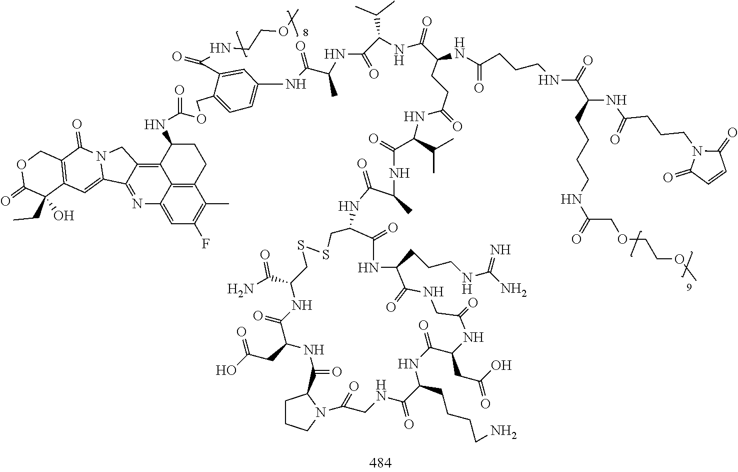

### Example 308: (37S,45S,50S,53S,56R,59S,65S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamoyl)oxy)methyl) phenyl)amino)-1-oxopropan-2-yl)amino)-3-methyl-1-oxobutan-2-yl)carbamoyl)-65-(((S)-6-amino-1-((2-((S)-2-(((S)-3-carboxy-1-(((R)-1-carboxy-2-mercaptoethyl)amino)-1-oxopropan-2-yl)carbamoyl) pyrrolidin-1-yl)-2-oxoethyl)amino)-1-oxohexan-2-yl)carbamoyl)-37-(4-(2,5-dioxo-2,5-dihydro-1H-pyrrol-1-yl)butanamido)-59-(3-guanidinopropyl)-50-isopropyl-56-(mercaptomethyl)-53-methyl-31,38,43,48,51,54,57,60,63-nonaoxo-2,5,8,11,14, 17,20,23,26,29-decaoxa-32,39,44,49,52,55,58,61,64-nonaazaheptahexacontan-67-oic acid (483)

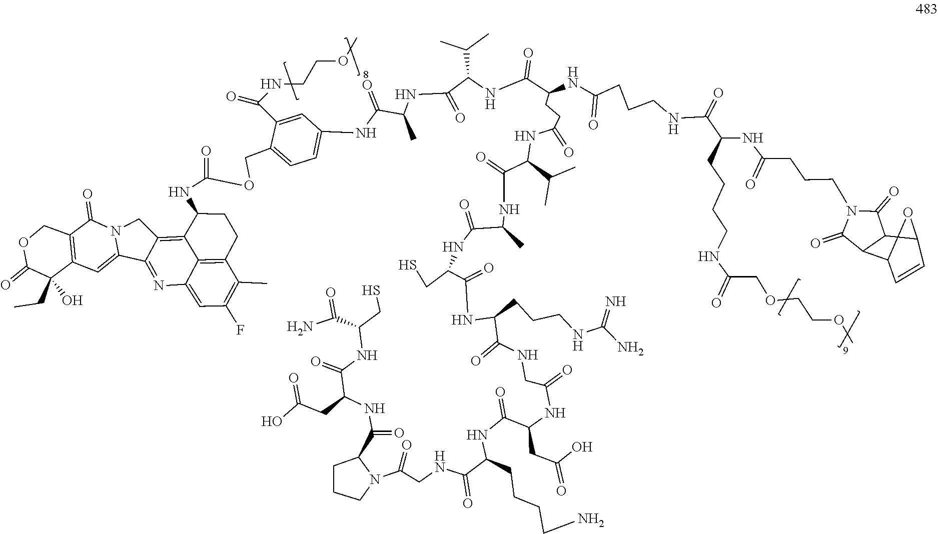

A peptide synthesis glass vessel (Chemgalss, 100-mL) were charged with 2-chlorotrityl chloride resin (3.00 g, 1.5 g/mmol), 480 (5.27 g, 9.900 mmol) dissolved in 50 mL of anhydrous DCM and DIEA (3.48 g, 14.850 mmol). The reaction was run overnight and the resin was washed with DCM (3×50 mL), followed by DMF (2×50 mL) to give compound 481.

The resulting resin-supported Fmoc-protected dipeptide was then sequentially deprotected and coupled with the third amino acid and so forth in an iterative fashion to give the desired resin-supported product 482.

A peptide synthesis glass vessel (Chemgalss, 100-mL) was charged with compound 482. Fmoc deprotection was performed using 10% piperidine/DMF solution (1000% v/w). The resin was then washed with DMF (1000% v/w) and then 452 (1.65 eq) dissolved in anhydrous DMF (1000% v/w), HOBt (1.65 eq) and DIC (1.65 eq) were added. The reaction was run for 4 h and the resin was then washed with DMF (3×1000% v/w). LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue, which was purified by prep-HPLC to give 483, MS ESI (m/z): calcd for C₁₄₅H₂₂₂FN₂₇O₅₁S2 [M+4H]⁴⁺: 827.89; found 827.89.

### Example 309:2,2′-((3S,6R,11R,14S,20S,23S,31aS)-11-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-50-isopropyl-53-methyl-31,38,43,48,51-pentaoxo-2,5,8, 11,14, 17,20,23,26,29-decaoxa-32,39,44,49,52-pentaazatetrapentacontan-54-amido)-23-(4-aminobutyl)-6-carboxy-14-(3-guanidinopropyl)-1,4,12, 15,18,21,24,27-octaoxooctacosahydro-1H-pyrrolo[2,1-j][1,2]dithia[5,8,11,14, 17,20,23,26]octaazacyclononacosine-3,20-diyl)diacetic acid (484)

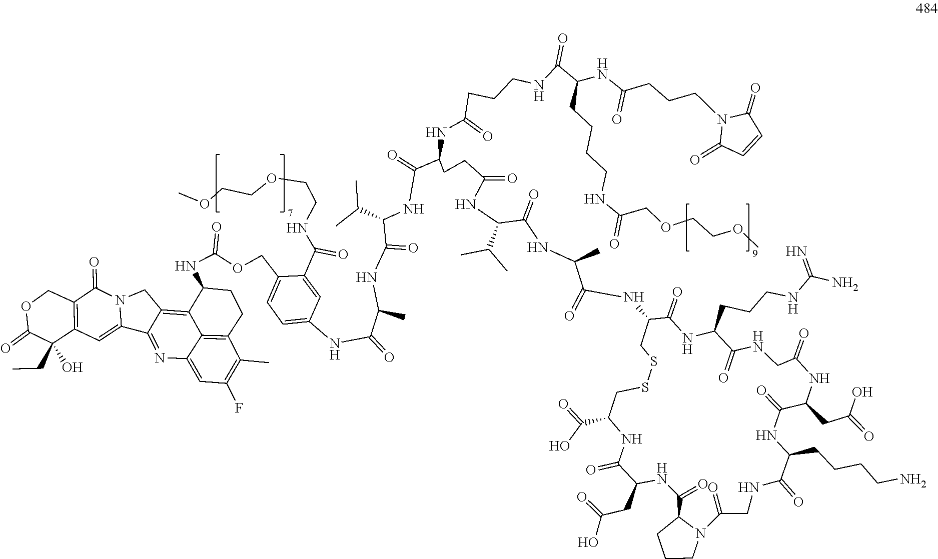

To a solution of compound 483 in MeOH (1000% v/w) was added water (1000% v/w). The mixture was stirred at room temperature overnight, and then concentrated under reduced pressure to give a residue, which was purified by prep-HPLC to give 484, MS ESI (m/z): calcd for C₁₄₅H₂₂₀FN₂₇O₅₁S2 [M+4H]⁴⁺: 810.62; found 810.62.

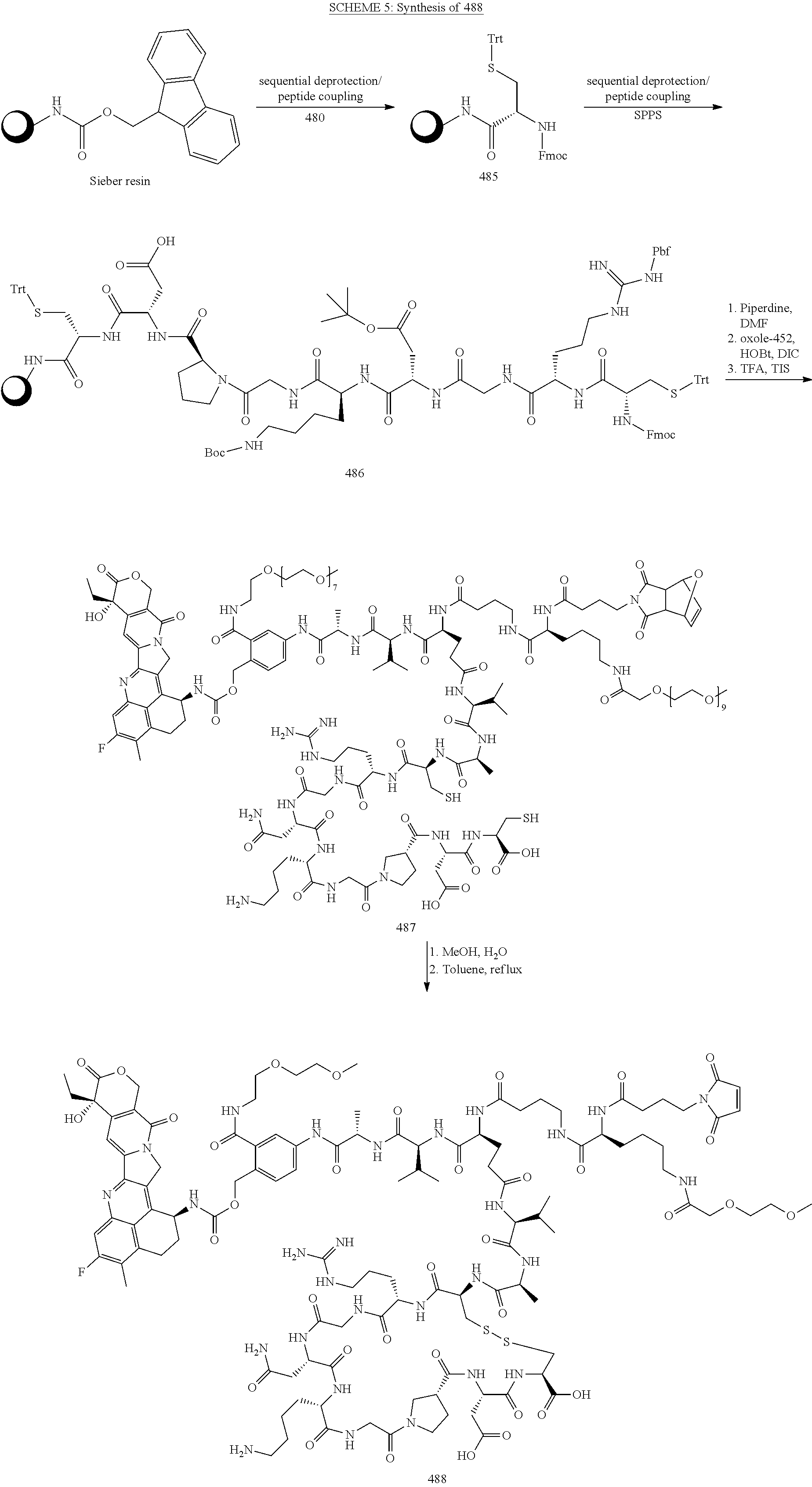

### Example 310: (37S,45S,50S,53S,56R,59S,65S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-65-(((S)-6-amino-1-((2-((S)-2-(((S)-1-(((R)-1-amino-3-mercapto-1-oxopropan-2-yl)amino)-3-carboxy-1-oxopropan-2-yl)carbamoyl) pyrrolidin-1-yl)-2-oxoethyl)amino)-1-oxohexan-2-yl)carbamoyl)-37-(4-(2,5-dioxo-2,5-dihydro-1H-pyrrol-1-yl)butanamido)-59-(3-guanidinopropyl)-53-isopropyl-56-(mercaptomethyl)-50-methyl-31,38,43,48,51,54,57,60,63-nonaoxo-2,5,8,11,14,17,20,23,26,29-decaoxa-32,39,44,49,52,55,58,61,64-nonaazaheptahexacontan-67-oic acid (487)

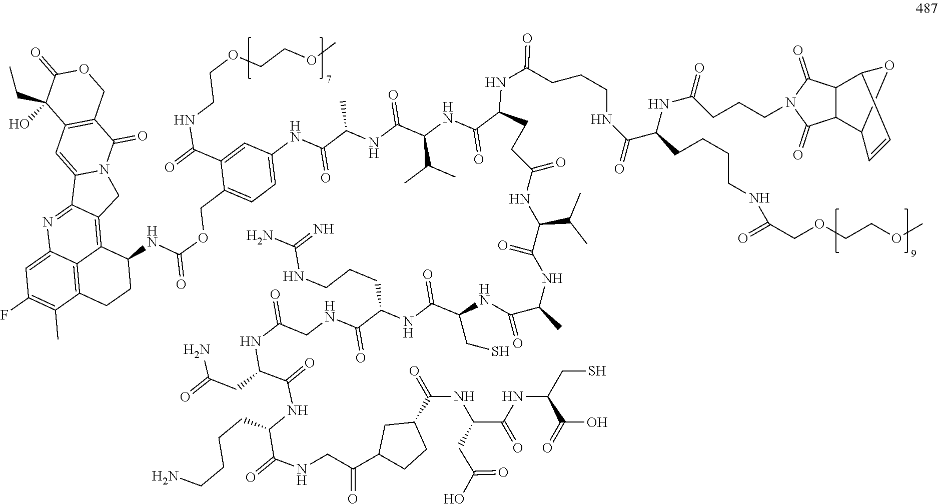

A peptide synthesis glass vessel (Chemgalss, 100-mL) was charged with Sieber resin (3.00 g, 1.5 g/mmol). The resin was treated with 20% piperidine/DMF solution (2×50 mL) and DMF (3×50 mL). A solution of 480 (5.27 g, 9.900 mmol), HATU (3.80 g, 9.900 mmol) and 4-Methylmorpholine (2.00 g, 9.900 mmol) in DMF (50 mL) was added. The reaction was run overnight and the resin was washed with DMF (2×50 mL) to give compound 485.

The resulting resin-supported Fmoc-protected dipeptide was then sequentially deprotected and coupled with the third amino acid and so forth in an iterative fashion to give the desired resin-supported product 486.

A peptide synthesis glass vessel (Chemgalss, 100-mL) was charged with compound 486. Fmoc deprotection was performed using 10% piperidine/DMF solution (1000% v/w). The resin was then washed with DMF (1000% v/w). 452 (1.65 eq) dissolved in 50 mL of anhydrous DMF and HOBt (1.65 eq), DIC (1.65 eq) were added. The reaction was run for 4 h and the resin was then washed with DMF (3×1000% v/w). LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue, which was purified by prep-HPLC to give 487. MS ESI (m/z): calcd for C₁₄₅H₂₂₀FN₂₇O₅₁S₂ [M+4H]⁴⁺: 810.88; found 810.88.

### Example 311:2,2′-((3S,6R,11R,14S,20S,23S,31aS)-11-((37S,45S,50S,53S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-53-isopropyl-50-methyl-31,38,43,48,51-pentaoxo-2,5,8,11,14,17,20,23,26,29-decaoxa-32,39,44,49,52-pentaazatetrapentacontan-54-amido)-23-(4-aminobutyl)-6-carbamoyl-14-(3-guanidinopropyl)-1,4,12,15,18,21,24,27-octaoxooctacosahydro-1H-pyrrolo[2,1-j][1,2]dithia[5,8,11,14,17,20,23,26]octaazacyclononacosine-3,20-diyl)diacetic acid (488)

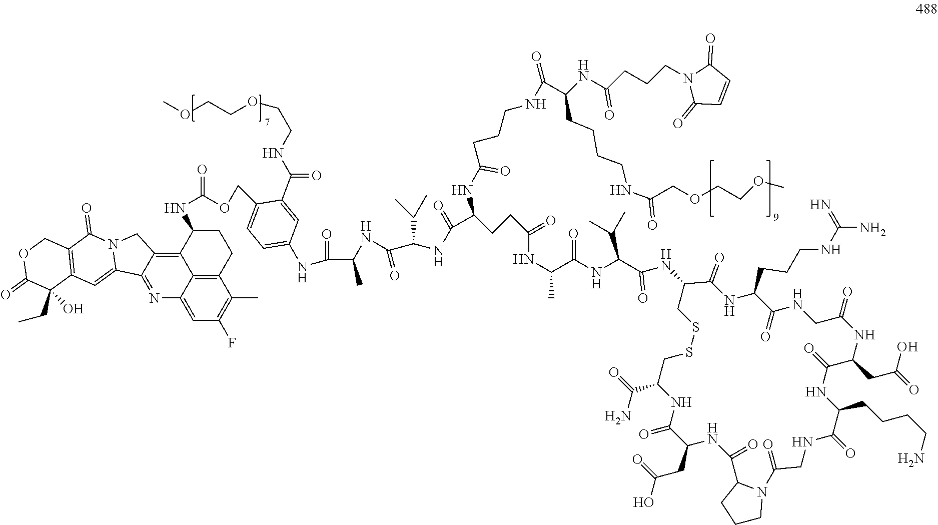

To a solution of compound 487 in MeOH (1000% v/w) was added water (1000% v/w). The mixture was stirred at room temperature overnight, and then concentrated under reduced pressure to give a residue, which was purified by prep-HPLC to give 488. MS ESI (m/z): calcd for C₁₄₅H₂₂₁FN₂₈O₅₀S₂ [M+4H]⁴⁺: 810.38; found 810.38.

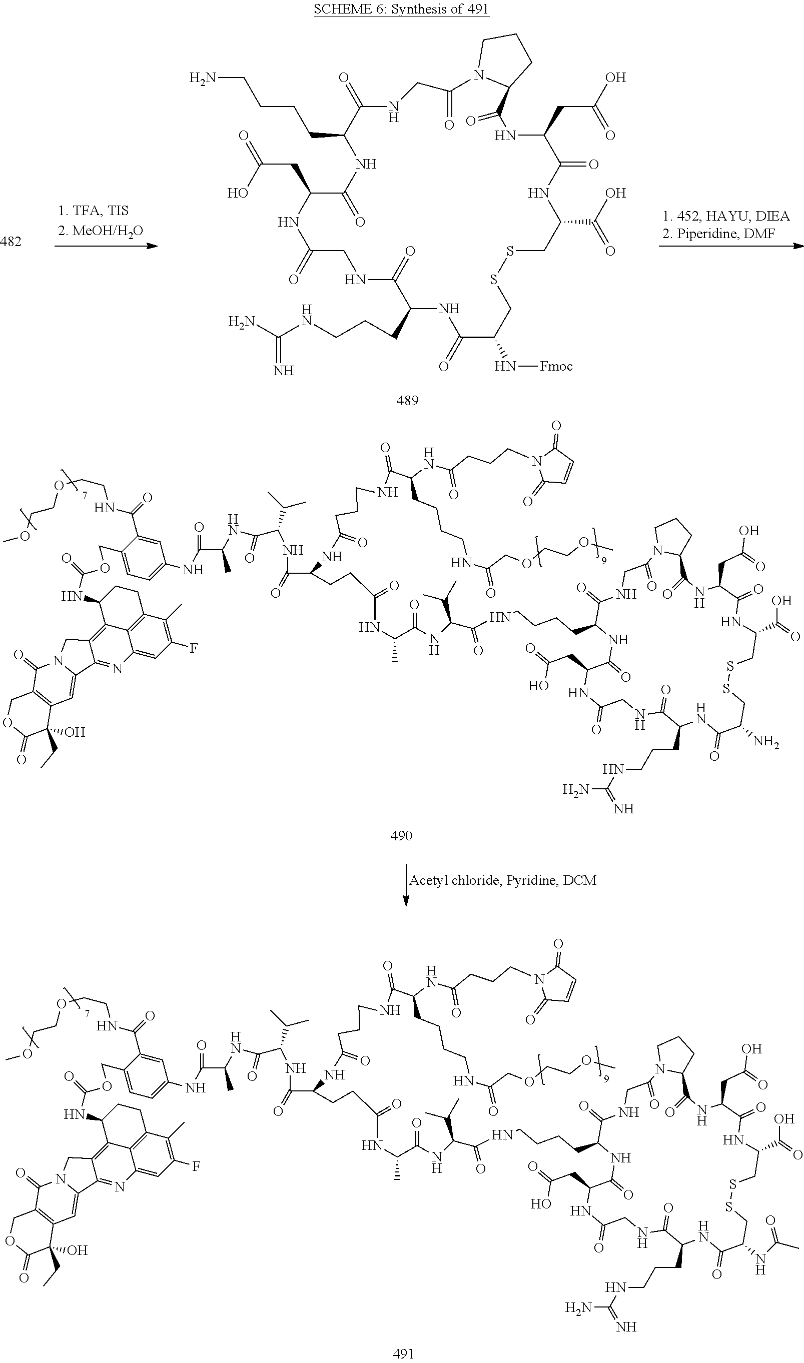

### Example 312:2,2′-((3S,6R,11R,14S,20S,23S,31aS)-23-((37S,45S,50S,53S)-45-(((S)-1-(((S)-1-((3-((2,5,8,11,14, 17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-53-isopropyl-50-methyl-31,38,43,48,51,54-hexaoxo-2,5,8,11,14,17,20,23,26,29-decaoxa-32,39,44,49,52,55-hexaazanonapentacontan-59-yl)-11-amino-6-carboxy-14-(3-guanidinopropyl)-1,4,12,15,18,21,24,27-octaoxooctacosahydro-1H-pyrrolo[2,1-j][1,2]dithia[5,8,11,14,17,20,23,26]octaazacyclononacosine-3,20-diyl)diacetic acid (490)

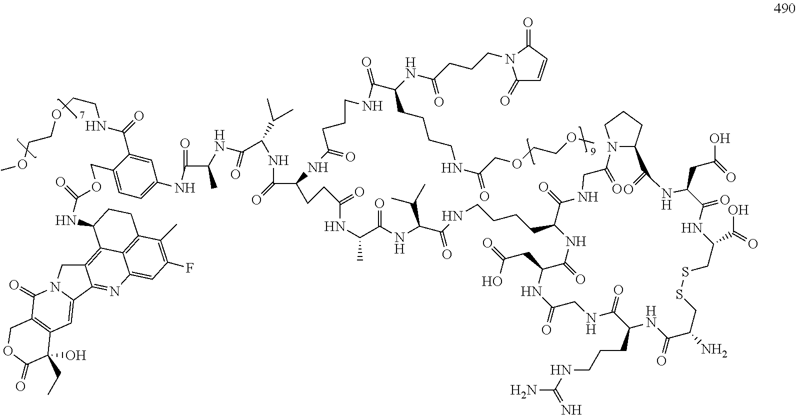

A peptide synthesis glass vessel (Chemgalss, 100-mL) was charged with compound 482. LCMS analysis was performed on a peptide aliquot, which was cleaved from the resin (analytical amount was treated with a TFA/TIS (96:4) solution (1000% v/w) at room temperature). The peptide-resin was washed with DCM (3×1000% v/w). The wash was concentrated under reduced pressure to give a residue, which was purified by prep-HPLC to give linear peptide. To the linear peptide in MeOH (1000% v/w) was added water (1000% v/w). The mixture was stirred at room temperature overnight and then concentrated under reduced pressure to give a residue, which was purified by prep-HPLC to give 489.

To a solution of 489 in DMF (1000% v/w) at room temperature were added 452 (1.00 eq), HATU (1.00 eq) and DIEA (2.00 eq). After stirring at room temperature for 1 h, the mixture was concentrated under reduced pressure to give a residue. Fmoc deprotection was performed using 10% piperidine/DMF solution (1000% v/w). After stirring at room temperature for 1 h, the mixture was concentrated under reduced pressure to give a residue, which was purified by prep-HPLC to give 490. MS ESI (m/z): calcd for C₁₄₅H₂₂₀FN₂₇O₅₁S2 [M+4H]⁴⁺: 810.62; found 810.62.

### Example 313:2,2′-((3S,6R,11R,14S,20S,23S,31aS)-23-((37S,45S,50S,53S)-45-(((S)-1-(((S)-1-((3-((2,5,8, 11,14, 17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-53-isopropyl-50-methyl-31,38,43,48,51,54-hexaoxo-2,5,8,11,14, 17,20,23,26,29-decaoxa-32,39,44,49,52,55-hexaazanonapentacontan-59-yl)-11-acetamido-6-carboxy-14-(3-guanidinopropyl)-1,4,12,15, 18,21,24,27-octaoxooctacosahydro-1H-pyrrolo[2,1-j][1,2]dithia[5,8,11,14, 17,20,23,26]octaazacyclononacosine-3,20-diyl)diacetic acid (491)

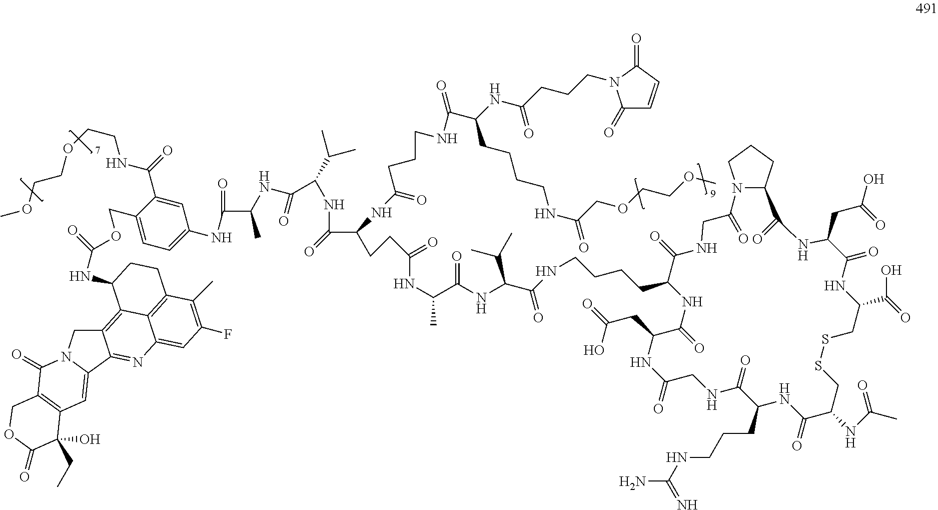

To a solution of compound 490 in DCM (1000% v/w) at room temperature were added pyridine (4.00 eq) and acetyl chloride (2.00 eq) in sequence. The mixture was concentrated under reduced pressure to give a residue, which was purified by prep-HPLC to give 491. MS ESI (m/z): calcd for C₁₄₇H₂₂₂FN₂₇O₅₂S2 [M+4H]⁴⁺: 821.12; found 821.11.

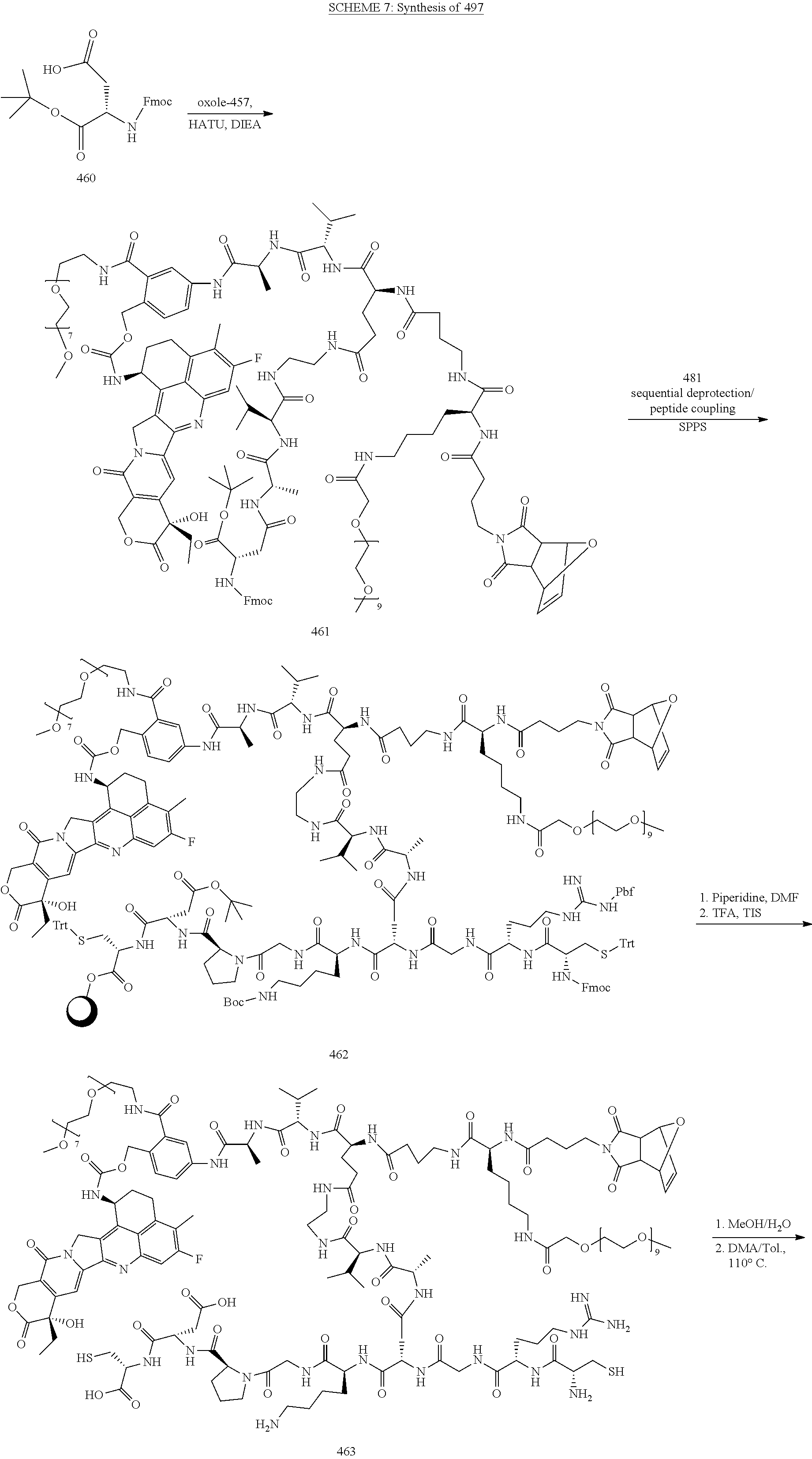

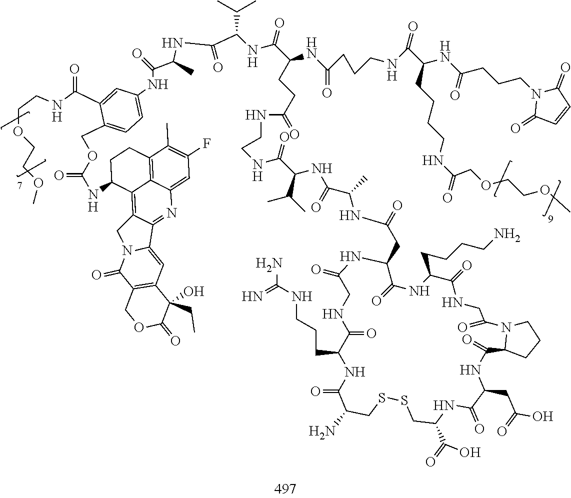

### Example 314: (3S,6R,11R,14S,20S,23S,31aS)-20-((37S,45S,54S,57S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9, 10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-54-isopropyl-57-methyl-31,38,43,48,53,56,59-heptaoxo-2,5,8,11,14,17,20,23,26,29-decaoxa-32,39,44,49,52,55,58-heptaazahexacontan-60-yl)-11-amino-23-(4-aminobutyl)-3-(carboxymethyl)-14-(3-guanidinopropyl)-1,4,12, 15, 18,21,24,27-octaoxooctacosahydro-1H-pyrrolo[2,1-j][1,2]dithia[5,8,11,14, 17,20,23,26]octaazacyclononacosine-6-carboxylic acid

To a solution of compound 457 (1.00 eq), compound 460 (1.50 eq) in DMF (1000% v/w) were added HATU (1.50 eq) and DIPEA (2.00 eq). The mixture was stirred at RT for 1 h, and then concentrated in vacuo. The residue was purified by prep-HPLC to afford 461.

The resulting resin-supported Fmoc-protected dipeptide was then sequentially deprotected and coupled with the third amino acid and so forth in an iterative fashion to give the desired resin-supported product 462.

A peptide synthesis glass vessel (Chemgalss, 100-mL) was charged with compound 482. Fmoc deprotection was performed using 10% piperidine/DMF solution (1000% v/w). The resin was then washed with DMF (1000% v/w). LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue, which was purified by prep-HPLC to give 463.

To a solution of compound 463 in MeOH (1000% v/w) was added water (1000% v/w). The mixture was stirred at room temperature overnight, and then concentrated under reduced pressure to give a residue, which was purified by prep-HPLC to give 497. MS ESI (m/z): calcd for C₁₄₇H₂₂₆FN₂₉O₅₀S2 [M+2H]₂₊: 1641.28: found 1641.11.

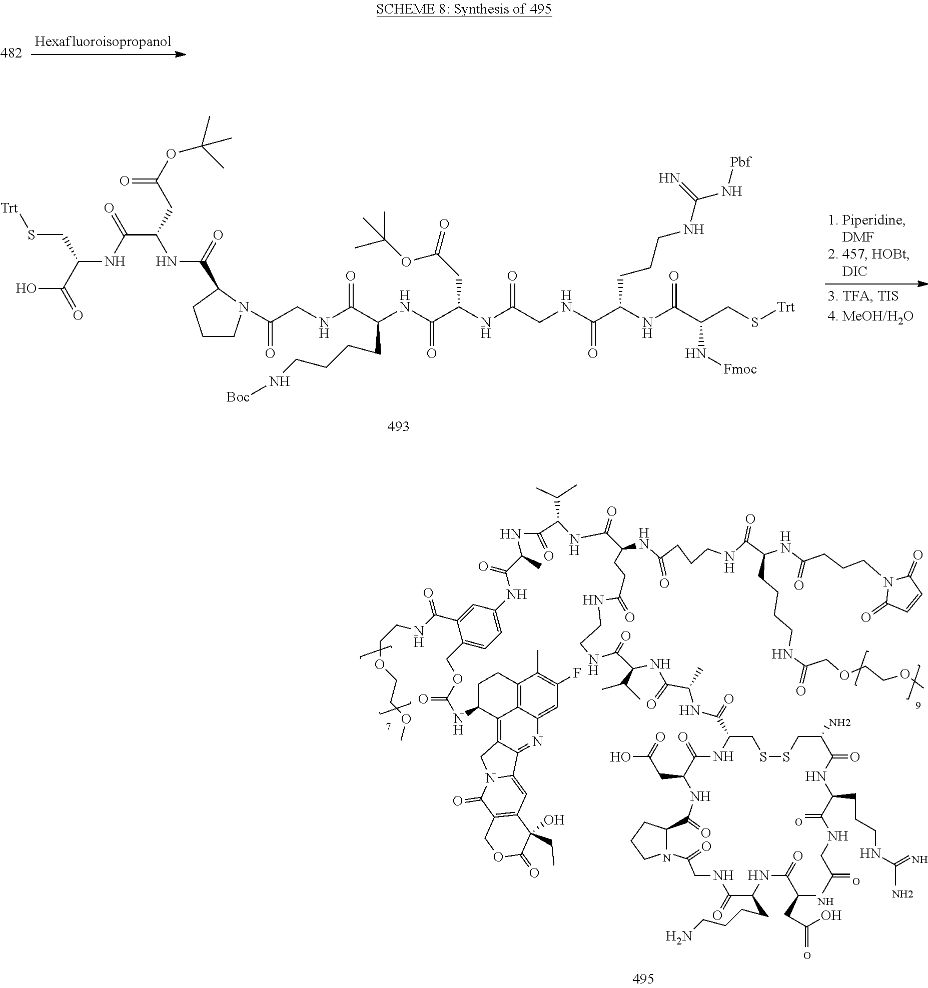

### Example 315: 2,2′-((3S,6R,11R,14S,20S,23S,31aS)-6-(((37S,45S,54S,57S)-45-(((S)-1-(((S)-1-((3-((2,5,8,11, 14, 17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-54-isopropyl-31,38,43,48,53,56-hexaoxo-2,5,8,11,14, 17,20,23,26,29-decaoxa-32,39,44,49,52,55-hexaazaoctapentacontan-57-yl)carbamoyl)-11-amino-23-(4-aminobutyl)-14-(3-guanidinopropyl)-1,4,12, 15,18,21,24,27-octaoxooctacosahydro-1H-pyrrolo[2,1-j][1,2]dithia[5,8,11,14,17,20,23,26]octaazacyclononacosine-3,20-diyl)diacetic acid (495)

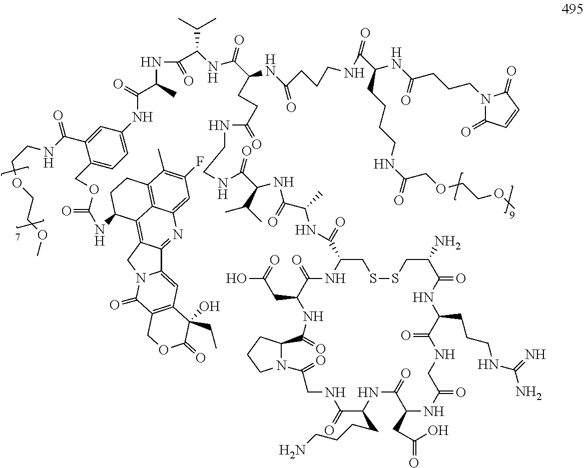

A peptide synthesis glass vessel (Chemgalss, 100-mL) was charged with compound 482. LCMS analysis was performed on a peptide aliquot, which was cleaved from the resin (analytical amount was treated with 10% 1,1,1,3,3,3-hexafluoropropan-2-ol/DCM (1000% v/w) at room temperature). The peptide-resin was washed with DCM (3×1000% v/w). The wash was concentrated under reduced pressure to give a residue, which was purified by prep-HPLC to give 493.

To a solution of peptide 493 in DMF (1000% v/w) at room temperature were added 457 (1.00 eq), HATU (1.00 eq) and DIEA (2.00 eq). After stirring at room temperature for 1 h, the mixture was concentrated under reduced pressure to give a residue. Fmoc deprotection was performed using 10% piperidine/DMF solution (1000% v/w). After stirring at room temperature for 1 h, the mixture was concentrated under reduced pressure to give a residue. And then MeOH (1000% v/w) and water (1000% v/w) were added. The mixture was stirred at room temperature overnight, and then concentrated under reduced pressure to give a residue, which was purified by prep-HPLC to give 495.

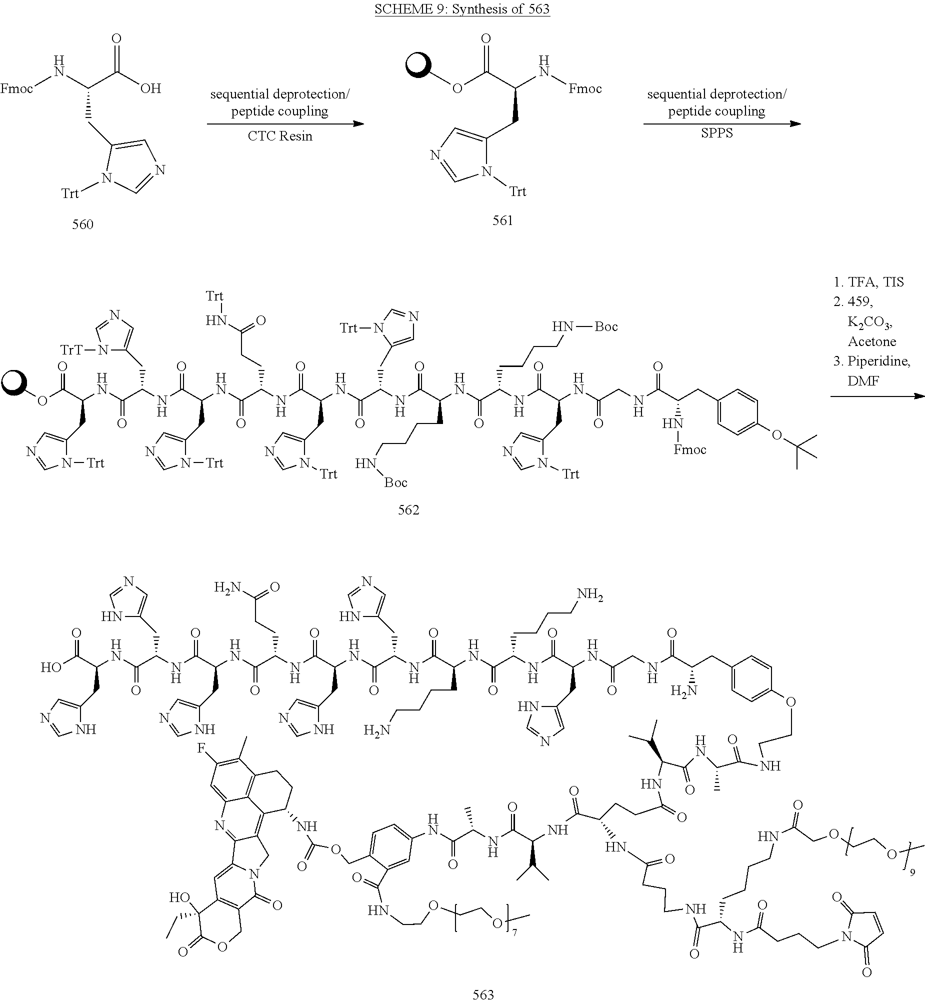

### Example 316: ((S)-3-(4-(2-((S)-2-((S)-2-((37S,45S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-amido)-3-methylbutanamido) propanamido)ethoxy)phenyl)-2-aminopropanoyl)glycyl-L-histidyl-L-lysyl-L-lysyl-L-histidyl-L-histidyl-L-glutaminyl-L-histidyl-L-histidyl-L-histidine (563)

A peptide synthesis glass vessel (Chemgalss, 100-mL) were charged with 2-chlorotrityl chloride resin (3.00 g, 1.5 g/mmol), 560 (6.13 g, 9.900 mmol) dissolved in 50 mL of anhydrous DCM and DIEA (3.48 g, 14.850 mmol). The reaction was run overnight and the resin was washed with DCM (3×50 mL), followed by DMF (2×50 mL) to give compound 561.

The resulting resin-supported Fmoc-protected dipeptide was then sequentially deprotected and coupled with the third amino acid and so forth in an iterative fashion to give the desired resin-supported product 562.

A peptide synthesis glass vessel (Chemgalss, 100-mL) was charged with compound 562. LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue. And then 459 (2.00 eq) dissolved in acetone (1000% v/w) and K₂CO₃ (1.65 eq) were added. The reaction was run overnight at 60° C. Fmoc deprotection was performed using 10% piperidine/DMF solution (1000% v/w). The mixture was concentrated under reduced pressure to give a residue, which was purified by prep-HPLC to give 563. MS ESI (m/z): calcd for C₁₇₆H₂₅₆FN₄₁O₅₁ [M+4H]⁴⁺: 945.72; found 945.72.

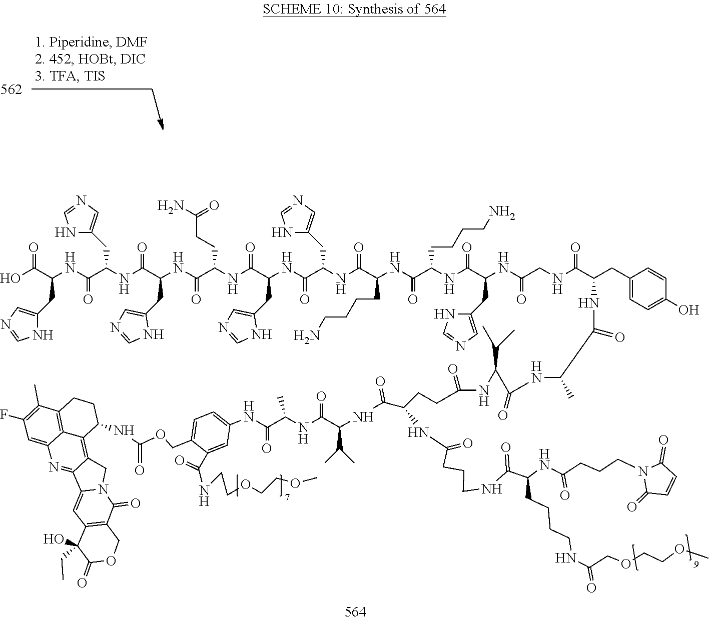

### Example 319: ((37S,45S)-45-(((S)-1-(((S)-1-((3-((2,5,8,11,14,17,20,23-octaoxapentacosan-25-yl) carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-31,38,43-trioxo-2,5,8,11,14,17,20,23,26,29-decaoxa-32,39,44-triazaoctatetracontan-48-oyl)-L-valyl-L-alanyl-L-tyrosylglycyl-L-histidyl-L-lysyl-L-lysyl-L-histidyl-L-histidyl-L-glutaminyl-L-histidyl-L-histidyl-L-histidine (564)

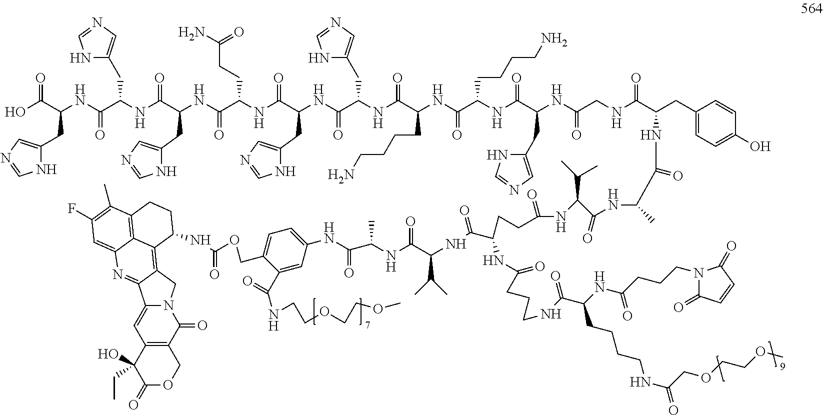

A peptide synthesis glass vessel (Chemgalss, 100-mL) was charged with compound 562. Fmoc deprotection was performed using 10% piperidine/DMF solution (1000% v/w). The resin was then washed with DMF (1000% v/w). And then 452 (1.65 eq) dissolved in anhydrous DMF (1000% v/w), HOBt (1.65 eq) and DIC (1.65 eq) were added. The reaction was run for 4 h, and the resin was then washed with DMF (3×1000% v/w). LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue, which was purified by prep-HPLC to give 564. MS ESI (m/z): calcd for C₁₇₄H₂₅₁FN₄₀O₅₁ [M+4H]⁴⁺: 934.96; found 934.96.

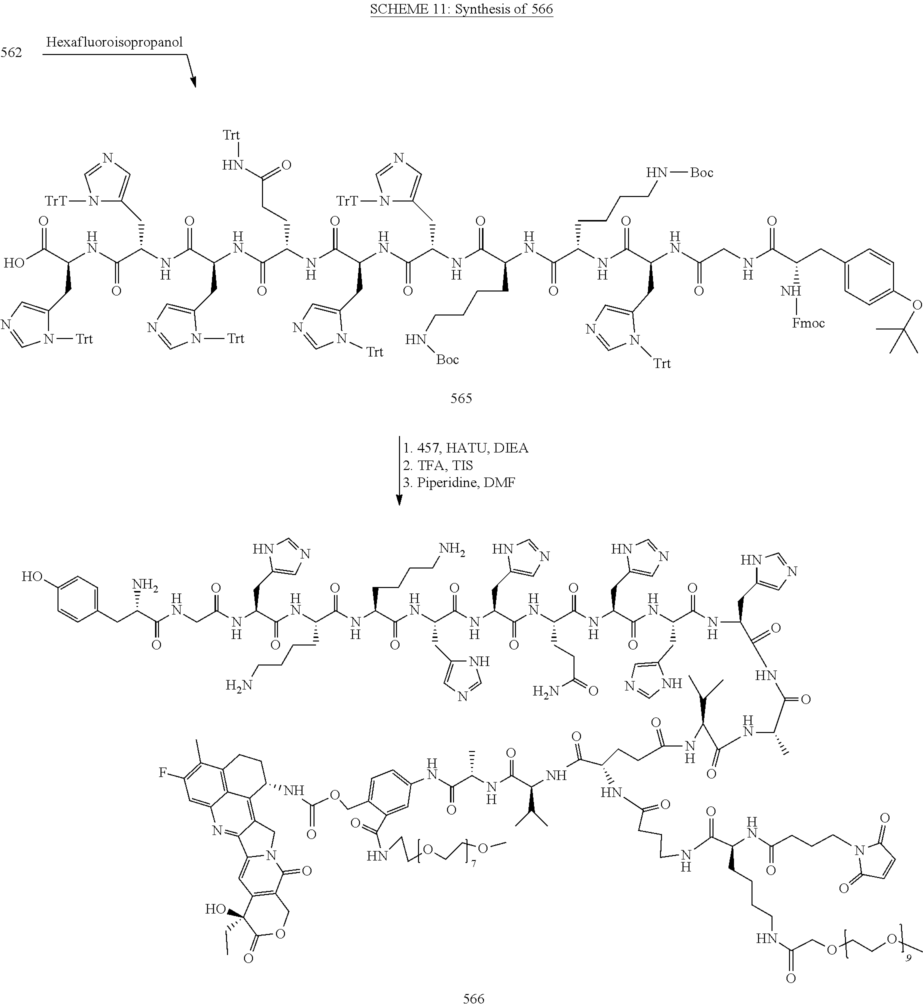

### Example 321: 2-((2,5,8,11,14, 17,20,23-octaoxapentacosan-25-yl)carbamoyl)-4-((37S,45S,48S,51S)-37-(4-(2,5-dioxo-2,5-dihydro-1H-pyrrol-1-yl)butanamido)-45-((5S,8S,12S,15S, 18S,21S,24S,27S,30S,33S,36S,42S)-12,15, 18,24,27,36-hexakis ((1H-imidazol-5-yl)methyl)-42-amino-21-(3-amino-3-oxopropyl)-30,33-bis(4-aminobutyl)-43-(4-hydroxyphenyl)-5-isopropyl-8-methyl-3,6,9,11,14,17,20,23,26,29,32,35,38,41-tetradecaoxo-4,7,10,13,16,19,22,25,28,31,34,37,40-tridecaazatritetracontyl)-48-isopropyl-51-methyl-31,38,43,46,49-pentaoxo-2,5,8,11,14,17,20,23,26,29-decaoxa-32,39,44,47,50-pentaazadopentacontan-52-amido)benzyl ((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl)carbamate (566)

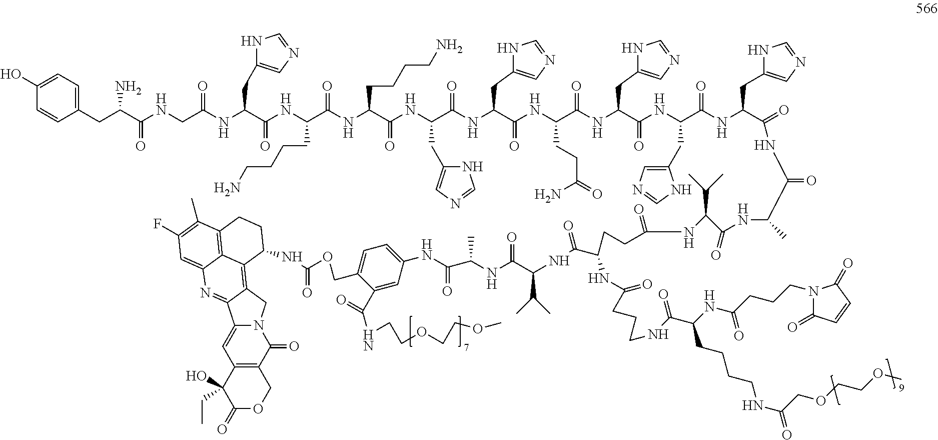

A peptide synthesis glass vessel (Chemgalss, 100-mL) was charged with compound 562. LCMS analysis was performed on a peptide aliquot, which was cleaved from the resin (analytical amount was treated with 10% 1,1,1,3,3,3-hexafluoropropan-2-ol/DCM (1000% v/w) at room temperature). The peptide-resin was washed with DCM (3×1000% v/w). The wash was concentrated under reduced pressure to give a residue, which was purified by prep-HPLC to give 565. To the linear peptide in DMF (1000% v/w) at room temperature were added 457 (1.00 eq), HATU (1.00 eq) and DIEA (2.00 eq). After stirring at room temperature for 1 h, the mixture was concentrated under reduced pressure to give a residue. Fmoc deprotection was performed using 10% piperidine/DMF solution (1000% v/w). After stirring at room temperature for 1 h, the mixture was concentrated under reduced pressure to give a residue, which was purified by prep-HPLC to give 566. MS ESI (m/z): calcd for C₁₇₄H₂₅₂FN₄₁O₅₁ [M+4H]⁴⁺: 819.71; found 819.71.

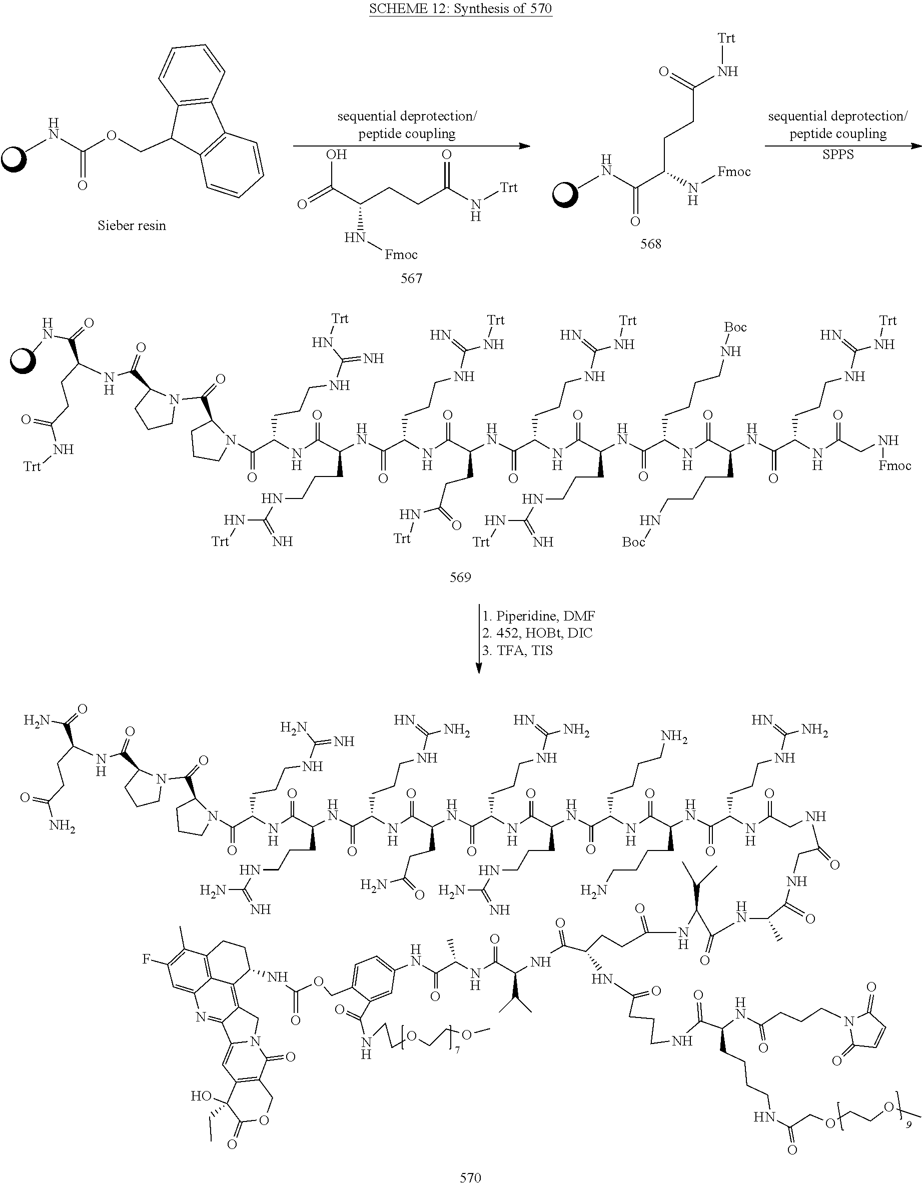

### Example 323: tert-butyl ((8S,11S,14S,17S,20S,23S,26S)-8-((S)-2-((S)-2-(((37S,45S,50S,53S,57S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9, 10,13, 15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-50-isopropyl-53-methyl-31,38,43,48,51,54,56,60-octaoxo-62,62,62-triphenyl-2,5,8,11,14,17,20,23,26,29-decaoxa-32,39,44,49,52,55,61-heptaazadohexacontan-57-yl)carbamoyl) pyrrolidine-1-carbonyl) pyrrolidine-1-carbonyl)-26-((8S,11S)-11-(4-((tert-butoxycarbonyl)amino)butyl)-1-(9H-fluoren-9-yl)-3,6,9-trioxo-8-(3-(3-tritylguanidino) propyl)-2-oxa-4,7,10-triazadodecan-12-amido)-3-imino-10,13,16,19,22,25-hexaoxo-17-(3-oxo-3-(tritylamino) propyl)-1,1,1-triphenyl-11,14,20,23-tetrakis (3-(3-tritylguanidino) propyl)-2,4,9,12, 15,18,21,24-octaazatriacontan-30-yl)carbamate (570)

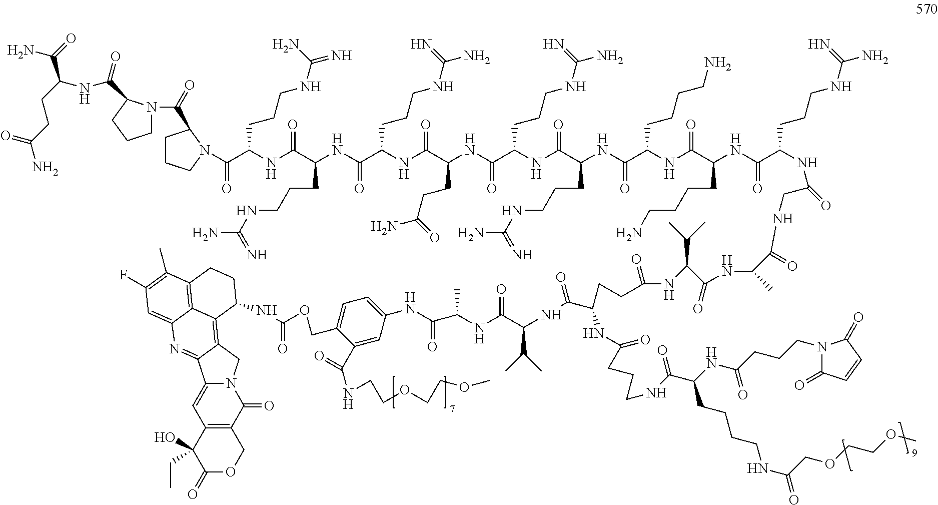

A peptide synthesis glass vessel (Chemgalss, 100-mL) was charged with Sieber resin (3.00 g, 1.5 g/mmol). The resin was treated with 20% piperidine/DMF solution (2×50 mL) and then washed with DMF (3×50 mL). A solution of 567 (6.04 g, 9.900 mmol), HATU (3.80 g, 9.900 mmol) and 4-Methylmorpholine (2.00 g, 9.900 mmol) in DMF (50 mL) was added. The reaction was run overnight and the resin was washed with DMF (2×50 mL) to give compound 568.

The resulting resin-supported Fmoc-protected dipeptide was then sequentially deprotected and coupled with the third amino acid and so forth in an iterative fashion to give the desired resin-supported product 569.

A peptide synthesis glass vessel (Chemgalss, 100-mL) was charged with compound 569. Fmoc deprotection was performed using 10% piperidine/DMF solution (1000% v/w). The resin was then washed with DMF (1000% v/w). And 452 (1.65 eq) dissolved in 50 mL of anhydrous DMF, HOBt (1.65 eq) and DIC (1.65 eq) were added. The reaction was run for 4 h, and the resin was then washed with DMF (3×1000% v/w). LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). The wash was concentrated under reduced pressure to give a residue, which was purified by prep-HPLC to give 570. MS ESI (m/z): calcd for C₁₈₀H₂₉₅FN₅O₀₅₂ [M+4H]⁴⁺: 1003.05; found 1003.05.

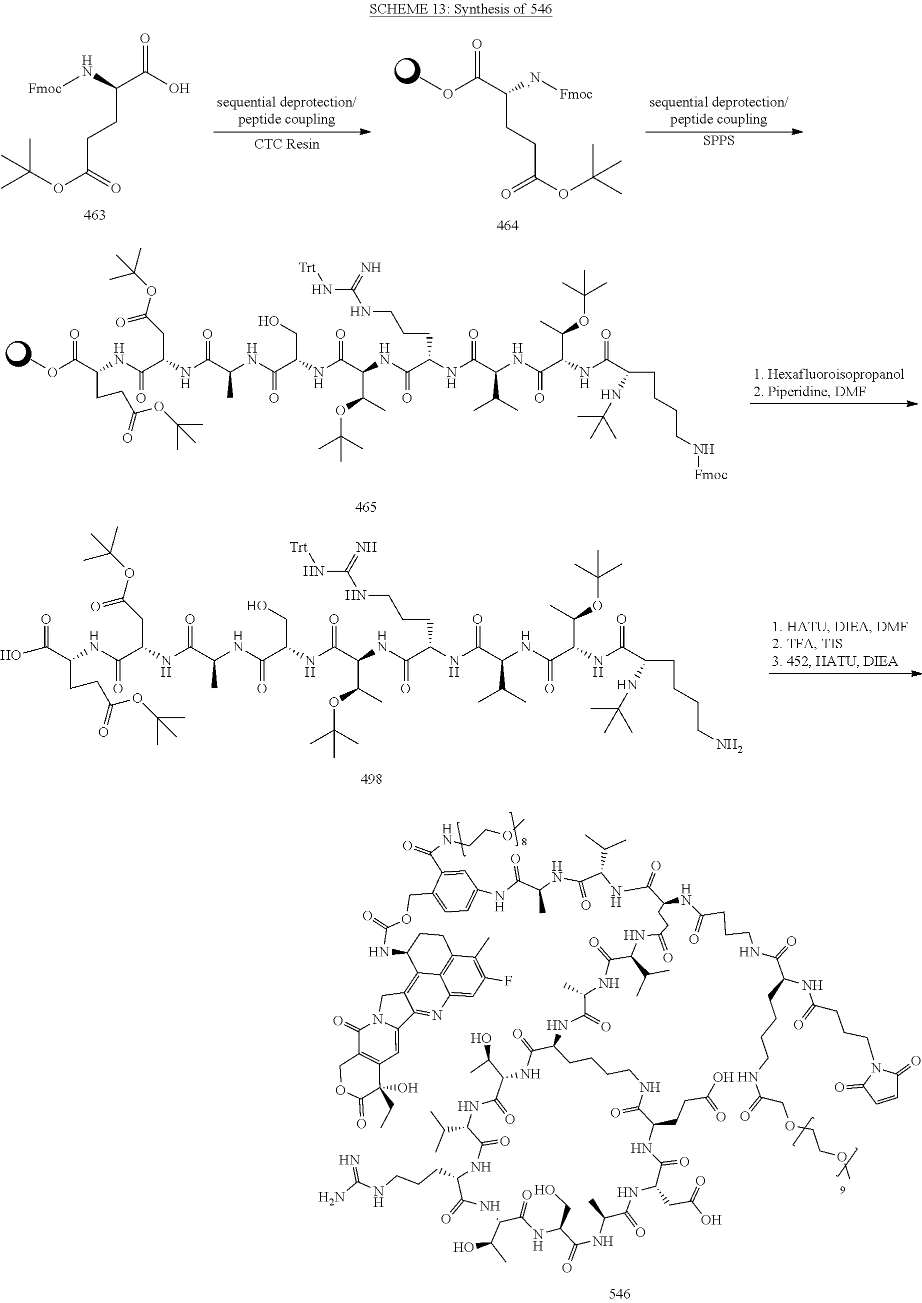

### Example 325: 3-((3R,6S,9S,12S,15S, 18S,21S,24S,27S)-27-((37S,45S,50S,53S)-45-(((S)-1-(((S)-1-((3-((2,5,8, 11, 14, 17,20,23-octaoxapentacosan-25-yl)carbamoyl)-4-(((((1S,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-1-oxopropan-2-yl)amino)-3-methyl-1-oxobutan-2-yl)carbamoyl)-37-(4-(2,5-dioxo-2,5-dihydro-1H-pyrrol-1-yl)butanamido)-50-isopropyl-53-methyl-31,38,43,48,51-pentaoxo-2,5,8,11,14, 17,20,23,26,29-decaoxa-32,39,44,49,52-pentaazatetrapentacontan-54-amido)-6-(carboxymethyl)-18-(3-guanidinopropyl)-15,24-bis((R)-1-hydroxyethyl)-12-(hydroxymethyl)-21-isopropyl-9-methyl-2,5,8,11,14,17,20,23,26-nonaoxo-1,4,7,10,13,16,19,22,25-nonaazacyclohentriacontan-3-yl) propanoic acid (546)

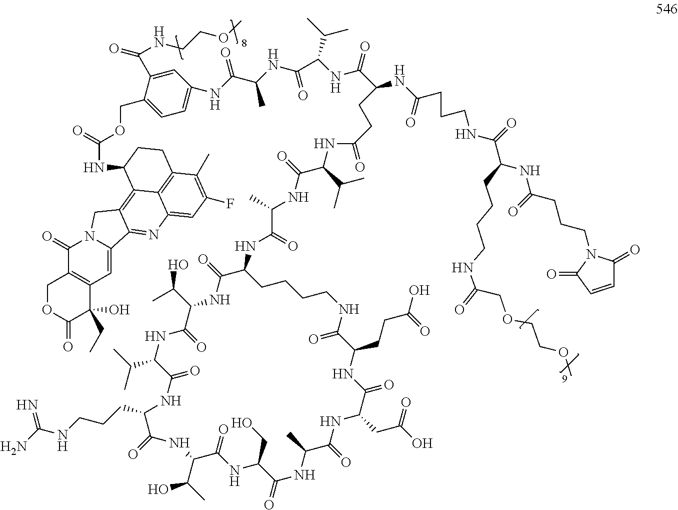

A peptide synthesis glass vessel (Chemgalss, 100-mL) were charged with 2-chlorotrityl chloride resin (3.00 g, 1.5 g/mmol), 463 (4.21 g, 9.900 mmol) dissolved in 50 mL of anhydrous DCM and DIEA (3.48 g, 14.850 mmol). The reaction was run overnight and the resin was washed with DCM (3×50 mL), followed by DMF (2×50 mL) to give compound 464.

The resulting resin-supported Fmoc-protected dipeptide was then sequentially deprotected and coupled with the third amino acid and so forth in an iterative fashion to give the desired resin-supported product 465.

Compound 465 was treated with 10% 1,1,1,3,3,3-hexafluoropropan-2-ol/DCM (1000% v/w) at room temperature. The peptide-resin was washed with DCM (3×1000% v/w). And the wash was concentrated under reduced pressure to give a residue. Fmoc deprotection was performed using 10% piperidine/DMF solution (1000% v/w). After stirring at room temperature for 1 h, the mixture was concentrated under reduced pressure to give a residue, which was purified by prep-HPLC to give 498.

To the linear peptide 498 in DMF (10000% v/w) at room temperature were added HATU (1.00 eq) and DIEA (2.00 eq). After stirring at room temperature for 1 h, the mixture was concentrated under reduced pressure to give a residue, to which TFA/TIS (96:4) solution (600% v/w) was added at room temperature. The mixture was stirred for 30 min and concentrated under reduced pressure to give a residue, which was purified by prep-HPLC to give 499. MS ESI (m/z): calcd for C₁₅₀H₂₃₂FN₂₇O₅₃ [M+2H]₂₊: 1641.31; found 1641.05.

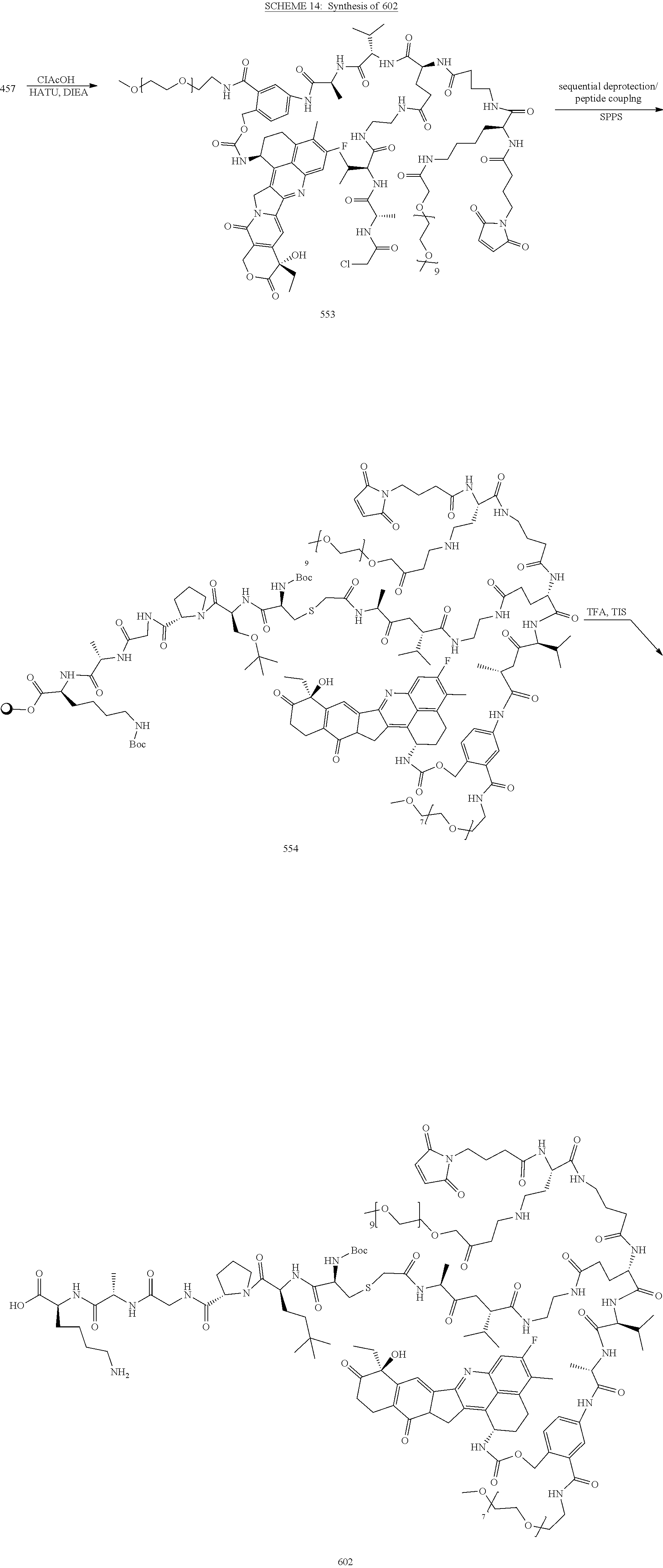

### Example 327: S-(2-(((S)-1-(((37S,45S,54S)-45-(((S)-1-(((S)-1-((3-((2,5,8,11,14,17,20,23-octaoxapentacosan-25-yl)carbamoyl)-4-(((((1S,9S)-9-ethyl-5-fluoro-9-hydroxy-4-methyl-10,13-dioxo-2,3,9,10,13, 15-hexahydro-1H, 12H-benzo[de]pyrano[3′,4′: 6,7]indolizino[1,2-b]quinolin-1-yl) carbamoyl)oxy)methyl)phenyl)amino)-1-oxopropan-2-yl)amino)-3-methyl-1-oxobutan-2-yl) carbamoyl)-37-(4-(2,5-dioxo-2,5-dihydro-1H-pyrrol-1-yl)butanamido)-55-methyl-31,38,43,48,53-pentaoxo-2,5,8,11,14,17,20,23,26,29-decaoxa-32,39,44,49,52-pentaazahexapentacontan-54-yl)amino)-1-oxopropan-2-yl)amino)-2-oxoethyl)-L-cysteinyl-L-seryl-L-prolylglycyl-L-alanyl-L-lysine (602)

To a solution of compound 457 (1.00 eq), 2-chloroacetic acid (1.50 eq) in DMF (1000% v/w) were added HATU (1.50 eq) and DIEA (2.00 eq). The mixture was stirred at RT for 1 h, and then concentrated in vacuo. The residue was purified by prep-HPLC to afford 553.

The resulting resin-supported Fmoc-protected dipeptide was then sequentially deprotected and coupled with the third amino acid and so forth in an iterative fashion to give the desired resin-supported product 554.

A peptide synthesis glass vessel (Chemgalss, 100-mL) was charged with compound 554. LCMS analysis was performed on a peptide aliquot, which was cleaved from the resin (analytical amount was treated with a TFA/TIS (96:4) solution (600% v/w) at room temperature). The peptide-resin was washed with DCM (3×1000% v/w). And the wash was concentrated under reduced pressure to give a residue, which was purified by prep-HPLC to give 602. MS ESI (m/z): calcd for C₁₃₆H₂₁₀FN₂₃O₄₆S [M+2H]₂₊: 1477.23; found 1477.58.

Example 328: 500, 503, 506, 509, 511, 514, 518, 520, 522, 525, 526, 527, 530, 532, 534, 535, 537, 540, 543, 547 were prepared following the general synthetic sequence described in SCHEME 4.
500: MS ESI (m/z): calcd for C₁₄₅H₂₂₀FN₂₉O₅₁S₂ [M+2H]₂₊: 1634.25; found 1634.52.
503: MS ESI (m/z): calcd for C₁₄₇H₂₃₀FN₃₁O₄₉S₂ [M+2H]₂₊: 1649.30; found 1649.55.
506: MS ESI (m/z): calcd for C₁₅₁H₂₃₂FN₃₁O₅₂S₂ [M+2H]₂₊: 1698.30; found 1698.69.
509: MS ESI (m/z): calcd for C₁₅₄H₂₄₃FN₃₄O₅₀S₂ [M+2H]₂₊: 1726.85; found 1726.99.
511: MS ESI (m/z): calcd for C₁₈₆H₂₆₇FN₃₂O₅₆S₂ [M+2H]₂₊: 1964.935; found 1965.35.
514: MS ESI (m/z): calcd for C₂₂₈H₃₅₉FN₆₁O₆₀ [M+2H]₂₊: 2465.84; found 2466.23.
518: MS ESI (m/z): calcd for C₁₅₃H₂₃₈FN₂₇O₅₀S₂ [M+2H]₂₊: 1669.32; found 1669.62.
520: MS ESI (m/z): calcd for C₁₉₁H₂₈₅FN₃₄O₆₁S₃ [M+2H]₂₊: 2073.97; found 2073.52.
522: MS ESI (m/z): calcd for C₁₄₈H₂₃₀FN₃₃O₅₁S₂ [M+2H]₂₊: 1685.29; found 1685.21.
525: MS ESI (m/z): calcd for C₂₆₂H₃₉₁FN₅₈O₇₅S₆ [M+3H]³⁺: 1925.67; found 1925.61.
526: MS ESI (m/z): calcd for C₂₆₂H₃₉₁FN₅₈O₇₅S₆ [M+3H]³⁺: 1925.67; found 1925.85.
527: MS ESI (m/z): calcd for C₁₆₅H₂₃₄FN₂₇O₄₉S₂ [M+2H]₂₊: 1731.31; found 1731.68.
530: MS ESI (m/z): calcd for C₁₅₄H₂₄₃FN₃₄O₅₀S₂ [M+2H]₂₊: 1726.85; found 1726.58.
532: MS ESI (m/z): calcd for C₁₉₃H₂₇₂FN₃₃O₅₇S₄ [M+2H]₂₊: 2063.41; found 2063.86.
534: MS ESI (m/z): calcd for C₁₉₂H₂₇₀FN₃₃O₅₇S₄ [M+2H]₂₊: 2049.41; found 2049.41.
535: MS ESI (m/z): calcd for C₁₅₃H₂₃₁FN₂₆O₄₉S₃ [M+2H]₂₊: 1666.78; found 1666.82.
537: MS ESI (m/z): calcd for C₁₄₈H₂₂₆FN₂₅O₅₃S₂ [M+2H]₂₊: 1643.26; found 1643.56.
540: MS ESI (m/z): calcd for C₁₅₆H₂₃₂FN₂₇O₅₂S₂ [M+2H]₂₊: 1700.29; found 1700.56.
543: MS ESI (m/z): calcd for C₁₇₅H₂₄₅FN₂₆O₅₇S₃ [M+2H]₂₊: 1869.81; found 1869.23.
547: MS ESI (m/z): calcd for C₁₄₃H₂₁₉FN₂₆O₅₂S₂ [M+2H]₂₊: 1608.74; found 1608.25.

Example 329:501, 508, 516 were prepared following the general synthetic sequence described in SCHEME 5.
501: MS ESI (m/z): calcd for C₁₄₅H₂₂₁FN₃₀O₅₀S₂ [M+2H]₂₊: 1633.76; found 1633.25.
508: MS ESI (m/z): calcd for C₂₂₈H₃₅₈FN₅₅O₆₄S₃ [M+3H]³⁺: 1669.52; found 1669.88.
516: MS ESI (m/z): calcd for C₂₁₆H₃₄₂FN₅₇O₆₂S₂ [M+2H]₂₊: 2405.74; found 2405.88.

Example 330:496, 502, 505, 510, 529, 541, 545, 615, 629, 630, 661, 672, 688, 693, 696 were prepared following the general synthetic sequence described in SCHEME 6.
496: MS ESI (m/z): calcd for C₁₄₆H₂₂₂FN₂₇O₅₁S₂ [M+2H]₂₊: 1627.25; found 1627.58.
502: MS ESI (m/z): calcd for C₁₄₇H₂₃₀FN₃₁O₄₉S₂ [M+2H]₂₊: 1649.30; found 1649.69.
505: MS ESI (m/z): calcd for C₁₅₁H₂₃₂FN₃₁O₅₂S₂ [M+2H]₂₊: 1698.30; found 1698.35.
510: MS ESI (m/z): calcd for C₁₅₄H₂₄₃FN₃₄O₅₀S₂ [M+2H]₂₊: 1726.85; found 1727.06.
529: MS ESI (m/z): calcd for C₁₅₄H₂₄₃FN₃₄O₅₀S₂ [M+2H]₂₊: 1726.85; found 1726.28.
541: MS ESI (m/z): calcd for C₁₅₆H₂₃₂FN₂₇O₅₂S₂ [M+2H]₂₊: 1700.29; found 1700.21.
545: MS ESI (m/z): calcd for C₁₆₆H₂₃₉FN₂₆O₄₇S₂ [M+2H]₂₊: 1716.83; found 1716.25.
615: MS ESI (m/z): calcd for C₁₄₈H₂₁₅FN₂₂O₄₄ [M+2H]₂₊: 1512.76; found 1512.75.
629: MS ESI (m/z): calcd for C₁₅₃H₂₃₈FN₃₁O₅₂S₂ [M+2H]₂₊: 1727.30; found 1727.25.
630: MS ESI (m/z): calcd for C₁₄₇H₂₃₂FN₃₁O₄₉S₂ [M+2H]₂₊: 1650.32; found 1650.85.
661: MS ESI (m/z): calcd for C₂₈₀H₄₅₃FN₆₄O₇₈S [M+3H]³⁺: 2004.77; found 2007.14.
672: MS ESI (m/z): calcd for C₂₃₄H₃₆₇FN₄₂O₇₃S [M+2H]₂₊: 2500.29; found 2500.59.
688: MS ESI (m/z): calcd for C₁₉₁H₂₈₄FN₃₃O₆₂S₂ [M+2H]₂₊: 2065.47; found 2065.86.
693: MS ESI (m/z): calcd for C₁₈₂H₃₀₁FN₃₄O₅₂S [M+2H]₂₊: 1908.10; found 1908.10.
696: MS ESI (m/z): calcd for C₁₉₇H₂₈₀FN₄₁O₅₉ [M+2H]₂₊: 2092.51; found 2092.62.

Example 331:515, 517, 524, 539 were prepared following the general synthetic sequence described in SCHEME 7.
515: MS ESI (m/z): calcd for C₁₇₁H₂₄₆F₂N₂₆O₅₀S₂ [M+2H]₂₊: 1783.85; found 1783.98.
517: MS ESI (m/z): calcd for C₂₁₈H₃₄₈FN₅₉O₆₁S₂ [M+2H]₂₊: 2426.77; found 2426.51.
524: MS ESI (m/z): calcd for C₁₅₀H₂₃₆FN₃₅O₅₀S₂ [M+2H]₂₊: 1706.32; found 1706.88.
539: MS ESI (m/z): calcd for C₁₅₂H₂₃₅FN₂₆O₅₂S₂ [M+2H]₂₊: 1670.80; found 1670.55.

Example 332:504, 507, 512, 519, 521, 523, 528, 531, 533, 536, 538, 542, 544 were prepared following the general synthetic sequence described in SCHEME 8.
504: MS ESI (m/z): calcd for C₁₄₉H₂₃₆FN₃₃O₄₈S₂ [M+2H]₂₊: 1670.33; found 1670.66.
507: MS ESI (m/z): calcd for C₁₅₃H₂₃₈FN₃₃O₅₁S₂ [M+2H]₂₊: 1719.33; found 1719.86.
512: MS ESI (m/z): calcd for C₁₈₈H₂₇₃FN₃₄O₅₅S₂ [M+2H]₂₊: 1985.95; found 1985.95.
519: MS ESI (m/z): calcd for C₁₅₅H₂₄₄FN₂₉O₄₉S₂ [M+2H]₂₊: 1690.35; found 1690.58.
521: MS ESI (m/z): calcd for C₁₉₃H₂₉₁FN₃₆O₆₀S₃ [M+2H]₂₊: 2095.00; found 2095.32.
523: MS ESI (m/z): calcd for C₁₅₀H₂₃₆FN₃₅O₅₀S₂ [M+2H]₂₊: 1706.32; found 1706.86.
528: MS ESI (m/z): calcd for C₁₆₇H₂₄₀FN₂₉O₄₈S₂ [M+2H]₂₊: 1731.31; found 1731.89.
531: MS ESI (m/z): calcd for C₁₅₆H₂₄₉FN₃₆O₄₉S₂ [M+2H]₂₊: 1747.38; found 1747.38.
533: MS ESI (m/z): calcd for C₁₉₅H₂₇₈FN₃₅O₅₆S₄ [M+2H]₂₊: 2084.44; found 2084.86.
536: MS ESI (m/z): calcd for C₁₅₅H₂₃₇FN₂₈O₄₈S₃ [M+2H]₂₊: 1687.81; found 1687.88.
538: MS ESI (m/z): calcd for C₁₅₂H₂₃₅FN₂₆O₅₂S₂ [M+2H]₂₊: 1670.80; found 1670.35.
542: MS ESI (m/z): calcd for C₁₅₈H₂₃₈FN₂₉O₅₁S₂ [M+2H]₂₊: 1721.32; found 1721.48.
544: MS ESI (m/z): calcd for C₁₇₇H₂₅₁FN₂₈O₅₆S₃ [M+2H]₂₊: 1890.84; found 1890.52.

Example 333:644 were prepared following the general synthetic sequence described in SCHEME 9.
644: MS ESI (m/z): calcd for C₁₉₄H₂₇₇FN₄₀O₅₉S₂ [M+2H]₂₊: 2111.95; found 2111.25.

Example 334:571, 573, 583, 585, 587, 590, 591, 593, 595, 597, 600, 603, 606, 608, 610, 612, 614, 620, 622, 624, 626, 631, 633, 635, 637, 639, 641, 643, 646, 648, 650, 652, 654, 657, 659, 663, 665, 667, 669, 671, 675, 677, 680, 682, 684, 686, 689, 692, 695, 698 were prepared following the general synthetic sequence described in SCHEME 10.
571: MS ESI (m/z): calcd for C₁₅₀H₂₂₅FN₂₆O₄₄ [M+2H]₂₊: 1573.80; found 1573.62.
573: MS ESI (m/z): calcd for C₁₆₇H₂₄₈FN₂₉O₄₈ [M+2H]₂₊: 1724.39 found 1724.86.
583: MS ESI (m/z): calcd for C₂₅₁H₃₅₂FN₆₂O₇₁S₂ [M+3H]³⁺: 1827.51; found 1827.86.
585: MS ESI (m/z): calcd for C₂₄₈H₃₇₉FN₅₆O₇₄ [M+3H]³⁺: 1801.57; found 1801.57.
587: MS ESI (m/z): calcd for C₂₅₅H₃₉₂FN₅₅O₇₁S₃ [M+3H]³⁺: 1830.92; found 1830.52.
590: MS ESI (m/z): calcd for C₁₈₃H₂₆₂FN₄₉O₅₄ [M+2H]₂₊: 2015.46 found 2015.25.
591: MS ESI (m/z): calcd for C₁₈₄H₂₅₅FN₅₂O₄₉ [M+2H]₂₊: 1998.95; found 1998.56.
593: MS ESI (m/z): calcd for C₂₄₁H₃₅₂FN₅₅O₆₈S [M+3H]³⁺: 1719.52; found 1719.56.
595: MS ESI (m/z): calcd for C₁₉₀H₂₇₀FN₃₉O₅₃S [M+2H]₂₊: 1999.47; found 1999.86.
597: MS ESI (m/z): calcd for C₂₅₄H₃₈₈FN₅₉O₇₉S₂ [M+3H]³⁺: 1880.91 found 1880.91.
600: MS ESI (m/z): calcd for C₁₃₃H₂₀₄FN₂₁O₅₃ [M+2H]₂₊: 1441.20; found 1441.32.
603: MS ESI (m/z): calcd for C₂₄₈H₃₈₄FN₄₉O₈₁ [M+3H]³⁺: 1788.91; found 1788.91.
606: MS ESI (m/z): calcd for C₁₄₄H₂₂₆FN₂₆O₄₉ [M+2H]₂₊: 1560.75; found 1560.92.
608: MS ESI (m/z): calcd for C₁₉₇H₂₉₂FN₃₇O₅₇S₂ [M+2H]₂₊: 2100.51; found 2100.89.
610: MS ESI (m/z): calcd for C₁₇₆H₂₆₅FN₃₀O₅₄S₂ [M+2H]₂₊: 1873.92; found 1873.53.
612: MS ESI (m/z): calcd for C₁₇₇H₂₆₆FN₃₅O₅₄ [M+2H]₂₊: 1883.46; found 1883.18.
614: MS ESI (m/z): calcd for C₁₆₀H₂₃₅FN₂₈O₄₈ [M+2H]₂₊: 1668.84; found 1668.32.
620: MS ESI (m/z): calcd for C₁₇₂H₂₅₃FN₃₂O₅₁ [M+2H]₂₊: 1801.91; found 1801.53.
622: MS ESI (m/z): calcd for C₂₁₆H₃₁₈FN₄₅O₆₇ [M+2H]₂₊: 2317.64; found 2317.46.
624: MS ESI (m/z): calcd for C₃₀₆H₄₅₉FN₆O₀₉₃S [M+3H]³⁺: 2181.42; found 2181.58.
626: MS ESI (m/z): calcd for C₂₀₉H₃₄₀FN₄₇O₆₄S [M+2H]₂₊: 2292.73; found 2292.63.
631: MS ESI (m/z): calcd for C₁₉₅H₃₀₅FN₄₂O₆₀ [M+2H]₂₊: 2108.11; found 2108.63.
633: MS ESI (m/z): calcd for C₁₈₇H₂₆₆FN₃₃O₅₅ [M+2H]₂₊: 1937.45; found 1937.03.
635: MS ESI (m/z): calcd for C₁₈₇H₂₆₅FN₃₂O₅₆ [M+2H]₂₊: 1937.95; found 1937.55.
637: MS ESI (m/z): calcd for C₁₉₁H₂₆₆FN₃₃O₅₆ [M+2H]₂₊: 1969.45; found 1969.58.
639: MS ESI (m/z): calcd for C₁₉₁H₂₆₅FN₃₂O₅₇ [M+2H]₂₊: 1969.94; found 1969.05.
641: MS ESI (m/z): calcd for C₁₈₂H₂₆₇FN₃₆O₅₇ [M+2H]₂₊: 1944.96; found 1944.58.
640: MS ESI (m/z): calcd for C₁₈₀H₂₆₁FN₃₄O₅₈ [M+2H]₂₊: 1923.93; found 1923.65.
643: MS ESI (m/z): calcd for C₁₉₃H₂₇₇FN₄₀O₅₇S₂ [M+2H]₂₊: 2089.96; found 2089.93.
646: MS ESI (m/z): calcd for C₁₉₉H₂₉₄FN₃₉O₅₅S [M+2H]₂₊: 2081.56; found 2081.58.
648: MS ESI (m/z): calcd for C₁₇₂H₂₅₆FN₃₇O₅₅ [M+2H]₂₊: 1870.42; found 1870.26.
650: MS ESI (m/z): calcd for C₁₈₉H₂₇₀FN₃₇O₅₆ [M+2H]₂₊: 1987.47; found 1987.25.
652: MS ESI (m/z): calcd for C₂₁₈H₃₄₇FN₄₄O₇₂S [M+2H]₂₊: 2400.22; found 2400.52.
654: MS ESI (m/z): calcd for C₂₉₄H₄₆₉FN₇₆O₈₉S [M+3H]³⁺: 2180.80; found 2181.52.
657: MS ESI (m/z): calcd for C₁₄₆H₂₃₅FN₃₂O₄₅S [M+2H]₂₊: 1611.83; found 1611.88.
659: MS ESI (m/z): calcd for C₂₆₈H₄₀₉FN₆O₀₇₄S₆ [M+3H]³⁺: 2011.25; found 2011.25.
663: MS ESI (m/z): calcd for C₂₂₉H₃₅₄FN₄₇O₆₇S [M+2H]₂₊: 2443.77; found 2443.25.
665: MS ESI (m/z): calcd for C₂₂₃H₃₅₄FN₄₇O₆₈S [M+2H]₂₊: 2399.79; found 2399.52.
667: MS ESI (m/z): calcd for C₂₂₁H₃₃₄FN₄₉O₅₅S [M+2H]₂₊: 2303.73; found 2303.28.
669: MS ESI (m/z): calcd for C₂₂₀H₃₅₀FN₄₅O₆₃S [M+2H]₂₊: 2342.76; found 2342.56.
671: MS ESI (m/z): calcd for C₂₃₆H₃₇₃FN₄₄O₇₂S [M+3H]³⁺: 1685.88; found 1685.25.
675: MS ESI (m/z): calcd for C₂₄₂H₃₇₂FN₅₃O₆₈S₂ [M+2H]₂₊: 1731.56; found 1731.25.
677: MS ESI (m/z): calcd for C₁₈₆H₂₆₇FN₃₀O₆₃S [M+2H]₂₊: 1990.92; found 1990.02.
680: MS ESI (m/z): calcd for C₁₇₀H₂₇₀FN₃₁O₅₁S [M+2H]₂₊: 1807.46; found 1807.98.
682: MS ESI (m/z): calcd for C₁₈₂H₂₆₂FN₃₇O₅₂ [M+2H]₂₊: 1909.45; found 1909.41.
684: MS ESI (m/z): calcd for C₂₄₆H₃₈₈FN₅₃O₆₈ [M+3H]³⁺: 1731.62; found 1732.99.
686: MS ESI (m/z): calcd for C₁₅₂H₂₄₇FN₃₀O₄₄ [M+2H]₂₊: 1608.91; found 1608.55.
689: MS ESI (m/z): calcd for C₁₉₃H₂₉₀FN₅O₆₁S₂ [M+2H]₂₊: 2086.50; found 2086.15.
692: MS ESI (m/z): calcd for C₁₈₄H₃₀₇FN₃₆O₅₁ [M+2H]₂₊: 1929.13; found 1929.85.
695: MS ESI (m/z): calcd for C₁₉₉H₂₈₆FN₄₃O₅₈ [M+2H]₂₊: 2113.54; found 2113.85.
698: MS ESI (m/z): calcd for C₁₄₄H₂₂₉FN₂₆O₄₆ [M+2H]₂₊: 1555.81; found 1555.26.

Example 335:574, 575, 582, 584, 586, 589, 592, 594, 596, 598, 601, 604, 605, 607, 609, 611, 613, 616, 617, 618, 619, 621, 623, 625, 628, 632, 634, 636, 638, 640, 642, 645, 647, 649, 651, 653, 655, 656, 658, 660, 662, 664, 666, 668, 670, 673, 674, 676, 678, 679, 681, 683, 685, 687.691, 694, 697 were prepared following the general synthetic sequence described in SCHEME 11.
574: MS ESI (m/z): calcd for C₁₆₂H₂₅₃FN₃₀O₄₆ [M+2H]₂₊: 1687.92; found 1687.95.
575: MS ESI (m/z): calcd for C₂₁₄H₃₁₂FN₄₃O₆₈ [M+2H]₂₊: 2296.62 found 2296.35.
582: MS ESI (m/z): calcd for C₂₄₉H₃₄₇FN₆O₀₇₂S₂ [M+3H]³⁺: 1813.49; found 1813.89.
584: MS ESI (m/z): calcd for C₂₄₆H₃₇₃FN₅₄O₇₅S [M+3H]³⁺: 1788.55; found 1788.55.
586: MS ESI (m/z): calcd for C₂₅₄H₃₈₈FN₅₃O₇₂S₃ [M+3H]³⁺: 1844.90; found 1844.56.
589: MS ESI (m/z): calcd for C₁₈₁H₂₅₆FN₄₇O₅₅ [M+2H]₂₊: 1993.44 found 1993.86.
592: MS ESI (m/z): calcd for C₁₈₂H₂₄₉FN₅O₀₅₀ [M+2H]₂₊: 1977.93; found 1978.35.
594: MS ESI (m/z): calcd for C₂₃₉H₃₄₆FN₅₃O₆₉S [M+3H]³⁺: 1705.50; found 1705.51.
596: MS ESI (m/z): calcd for C₁₈₈H₂₆₄FN₃₇O₅₄ [M+2H]₂₊: 1977.44; found 1977.44.
598: MS ESI (m/z): calcd for C₂₅₂H₃₈₂FN₅₇O₈₀S₂ [M+3H]³⁺: 1866.89 found 1866.89.
601: MS ESI (m/z): calcd for C₁₃₂H₂₀₀FN₂₁O₄₄S [M+2H]₂₊: 1427.20; found 1427.32.
604: MS ESI (m/z): calcd for C₂₄₆H₃₇₈FN₄₇O₈₂ [M+3H]³⁺: 1774.89; found 1774.82.
605: MS ESI (m/z): calcd for C₁₄₂H₂₁₇FN₂₄O₅₀ [M+2H]₂₊: 1539.76; found 1539.25.
607: MS ESI (m/z): calcd for C₁₉₃H₂₈₂FN₃₅O₅₈S₂ [M+2H]₂₊: 2051.48; found 2051.86.
609: MS ESI (m/z): calcd for C₁₇₄H₂₅₉FN₂₈O₅₅S₂ [M+2H]₂₊: 1852.89; found 1852.91.
611: MS ESI (m/z): calcd for C₁₇₅H₂₆₀FN₃₃O₅₅ [M+2H]₂₊: 1862.43; found 1862.91.
613: MS ESI (m/z): calcd for C₁₅₈H₂₂₉FN₂₆O₄₉ [M+2H]₂₊: 1647.81; found 1647.25.
616: MS ESI (m/z): calcd for C₁₈₇H₂₇₁FN₃₂O₅₆ [M+2H]₂₊: 1979.93; found 1979.25.
617: MS ESI (m/z): calcd for C₁₆₈H₂₄₆FN₃₁O₅₀ [M+2H]₂₊: 1759.38; found 1759.68.
618: MS ESI (m/z): calcd for C₁₆₉H₂₄₇FN₃₂O₅₁ [M+2H]₂₊: 1780.89; found 1780.56.
619: MS ESI (m/z): calcd for C₁₇₀H₂₅₀FN₃₁O₅₀ [M+2H]₂₊: 1773.40; found 1773.91.
621: MS ESI (m/z): calcd for C₂₁₄H₃₁₂FN₄₃O₆₈ [M+2H]₂₊: 2296.62; found 2296.68.
623: MS ESI (m/z): calcd for C₃₀₄H₄₅₃FN₅₈O₉₄ [M+3H]³⁺: 2167.40; found 2167.42.
625: MS ESI (m/z): calcd for C₂₀₇H₃₃₄FN₄₅O₆₅S [M+2H]₂₊: 2271.70; found 2271.36.
628: MS ESI (m/z): calcd for C₁₉₃H₂₉₉FN₄₀O₆₁ [M+2H]₂₊: 2086.08; found 2086.58.
632: MS ESI (m/z): calcd for C₁₈₅H₂₆₀FN₃₁O₅₆ [M+2H]₂₊: 1916.42; found 1916.08.
634: MS ESI (m/z): calcd for C₁₈₅H₂₅₉FN₃₀O₅₇ [M+2H]₂₊: 1916.92; found 1916.68.
636: MS ESI (m/z): calcd for C₁₈₉H₂₆₀FN₃₁O₅₇ [M+2H]₂₊: 1948.42; found 1948.42.
638: MS ESI (m/z): calcd for C₁₈₉H₂₅₉FN₃₀O₅₈ [M+2H]₂₊: 1948.91; found 1948.23.
640: MS ESI (m/z): calcd for C₁₈₀H₂₆₁FN₃₄O₅₈ [M+2H]₂₊: 1923.93; found 1923.65.
642: MS ESI (m/z): calcd for C₁₉₂H₂₇₁FN₃₈O₅₈S₂ [M+2H]₂₊: 2068.93; found 2068.75.
645: MS ESI (m/z): calcd for C₁₉₈H₂₉₀FN₃₇O₅₆S [M+2H]₂₊: 2074.53; found 2074.52.
647: MS ESI (m/z): calcd for C₁₇₀H₂₅₀FN₃₅O₅₆ [M+2H]₂₊: 1849.39; found 1849.86.
649: MS ESI (m/z): calcd for C₁₈₇H₂₆₄FN₃₅O₅₇ [M+2H]₂₊: 1966.44; found 1966.85.
651: MS ESI (m/z): calcd for C₂₁₆H₃₄₁FN₄₂O₇₃S [M+2H]₂₊: 2379.19; found 2379.32.
653: MS ESI (m/z): calcd for C₂₉₂H₄₆₃FN₇₄O₉₀S [M+3H]³⁺: 2166.79; found 2166.89.
655: MS ESI (m/z): calcd for C₁₅₅H₂₄₄FN₂₇O₅₀S₂ [M+2H]₂₊: 1698.33; found 1698.85.
656: MS ESI (m/z): calcd for C₁₄₄H₂₂₉FN₃₀O₄₅S [M+2H]₂₊: 1590.81; found 1590.98.
658: MS ESI (m/z): calcd for C₂₆₆H₄₀₃FN₅₈O₇₅S₆ [M+3H]³⁺: 1997.23; found 1997.86.
660: MS ESI (m/z): calcd for C₂₈₀H₄₅₃FN₆₄O₇₈S [M+3H]³⁺: 2004.77; found 2007.14.
662: MS ESI (m/z): calcd for C₂₂₇H₃₄₈FN₄₅O₆₈S [M+2H]₂₊: 2422.75; found 2422.56.
664: MS ESI (m/z): calcd for C₂₂₁H₃₄₈FN₄₅O₆₉S [M+2H]₂₊: 2378.76; found 2378.79.
666: MS ESI (m/z): calcd for C₂₁₉H₃₂₈FN₄₇O₅₆S [M+2H]₂₊: 2282.70; found 2282.52.
668: MS ESI (m/z): calcd for C₂₁₇H₃₄₄FN₄₃O₆₄S [M+2H]₂₊: 2321.73; found 2321.74.
670: MS ESI (m/z): calcd for C₂₃₄H₃₆₇FN₄₂O₇₃S [M+2H]₂₊: 2500.29; found 2500.38.
673: MS ESI (m/z): calcd for C₁₆₁H₂₃₃FN₂₄O₄₈S₂ [M+2H]₂₊: 1691.75; found 1691.25.
674: MS ESI (m/z): calcd for C₂₄₀H₃₆₆FN₅₁O₆₉S₂ [M+3H]³⁺: 1717.54; found 1717.55.
676: MS ESI (m/z): calcd for C₁₈₄H₂₆₁FN₂₈O₆₄S [M+2H]₂₊: 1969.89; found 1969.25.
678: MS ESI (m/z): calcd for C₁₉₂H₂₇₂FN₃₃O₅₇S₄ [M+2H]₂₊: 2050.42; found 2050.42.
679: MS ESI (m/z): calcd for C₁₆₈H₂₆₄FN₂₉O₅₂S [M+2H]₂₊: 1786.43; found 1786.38.
681: MS ESI (m/z): calcd for C₁₈₀H₂₅₆FN₃₅O₅₃S [M+2H]₂₊: 1888.49; found 1888.25.
683: MS ESI (m/z): calcd for C₂₄₄H₃₈₂FN₅₁O₆₉ [M+3H]³⁺: 171760; found 1717.25.
685: MS ESI (m/z): calcd for C₁₅₀H₂₄₁FN₂₈O₄₅ [M+2H]₂₊: 1587.87; found 1587.95.
687: MS ESI (m/z): calcd for C₁₉₁H₂₈₄FN₃₃O₆₂S₂ [M+2H]₂₊: 2065.47; found 2065.96.
691: MS ESI (m/z): calcd for C₁₈₂H₃₀₁FN₃₄O₅₂ [M+2H]₂₊: 1908.10; found 1908.68.
694: MS ESI (m/z): calcd for C₁₉₇H₂₈₀FN₄₁O₅₉ [M+2H]₂₊: 2092.51; found 209286.
697: MS ESI (m/z): calcd for C₁₄₂H₂₂₃FN₂₄O₄₇S [M+2H]₂₊: 1534.78; found 1534.89.

Example 336:576, 577, 578, 579, 580, 581 were prepared following the general synthetic sequence described in SCHEME 12.
576: MS ESI (m/z): calcd for C₁₈₀H₂₉₅FN₅O₀₅₂ [M+2H]₂₊: 2005.10; found 2005.10.
577: MS ESI (m/z): calcd for C₁₇₄H₂₈₂FN₄₇O₅₀ [M+2H]₂₊: 1925.55 found 1925.68.
578: MS ESI (m/z): calcd for C₂₁₄H₃₃₂FN₄₉O₅₆S [M+2H]₂₊: 2268.72; found 2268.52.
579: MS ESI (m/z): calcd for C₁₈₆H₃₀₇FN₅₂O₅₂ [M+2H]₂₊: 2069.15; found 2069.56.
580: MS ESI (m/z): calcd for C₁₈₈H₃₁₈FN₆₁O₅₃ [M+2H]₂₊: 2149.70; found 2149.25.
581: MS ESI (m/z): calcd for C₁₅₉H₂₆₁FN₄₂O₄₇ [M+2H]₂₊: 1765.97; found 1765.25

Example 337:546, 548, 549, 550 were prepared following the general synthetic sequence described in SCHEME 13.
546: MS ESI (m/z): calcd for C₁₅₀H₂₃₂FN₂₇O₅₃ [M+2H]₂₊: 1640.32; found 1640.25.
548: MS ESI (m/z): calcd for C₁₄₉H₂₃₁FN₂₈O₅₂ [M+2H]₂₊: 1632.82 found 1632.25.
549: MS ESI (m/z): calcd for C₁₅₀H₂₃₂FN₂₇O₅₃ [M+2H]₂₊: 1640.32; found 1640.32.
550: MS ESI (m/z): calcd for C₁₅₀H₂₃₃FN₂₈O₅₂ [M+2H]₂₊: 1639.82; found 1639.56.

Example 338:513, 552, 627, 690 were prepared following the general synthetic sequence described in SCHEME 14.
513: MS ESI (m/z): calcd for C₁₅₀H₂₂₁FN₂₄O₄₅S [M+2H]₂₊: 1565.78; found 1565.98.
552: MS ESI (m/z): calcd for C₁₃₆H₂₀₈FN₂₃O₄₅S [M+2H]₂₊: 1469.22; found 1468.22.
627: MS ESI (m/z): calcd for C₂₁₁H₃₄₂FN₄₇O₆₆S [M+2H]₂₊: 2321.73; found 2321.58.
690: MS ESI (m/z): calcd for C₁₉₄H₂₉₀FN₃₅O₆₃S₂ [M+2H]₂₊: 2101.50; found 2101.23.

### Example 350: Synthesis of A001

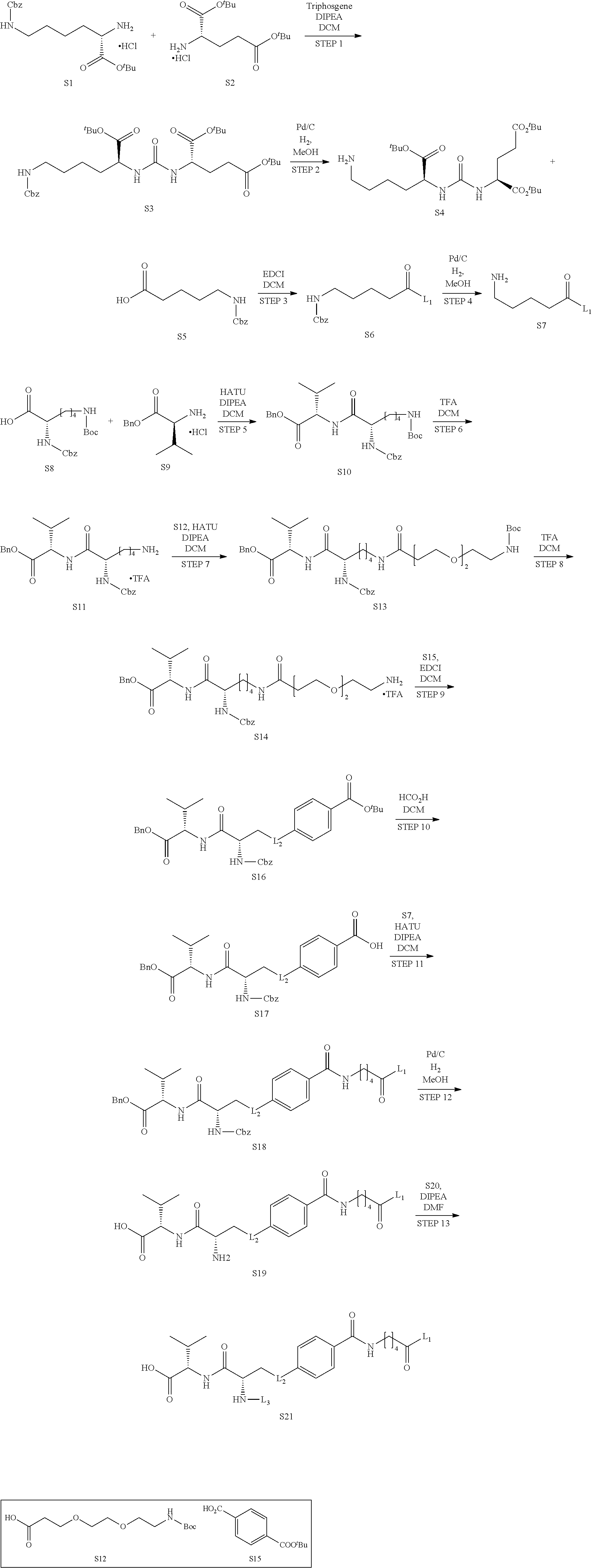

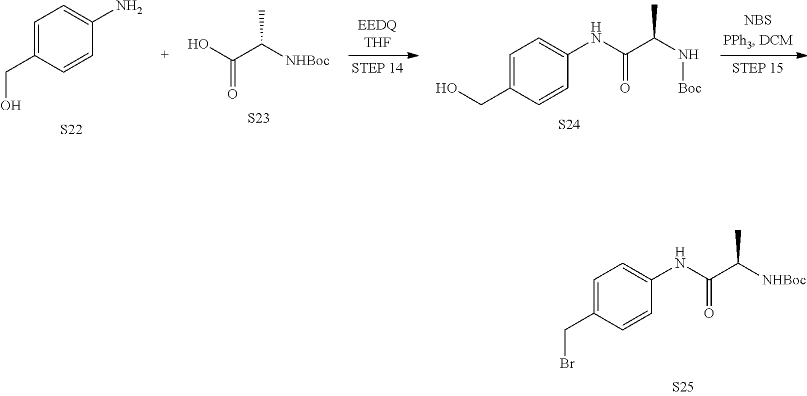 [SMILES: Nc1ccc(CO)cc1 ; C[C@H](NC(=O)OC(C)(C)C)C(=O)O ; C[C@@H](NC(=O)OC(C)(C)C)C(=O)Nc1ccc(CO)cc1 ; C[C@@H](NC(=O)OC(C)(C)C)C(=O)Nc1ccc(CBr)cc1]

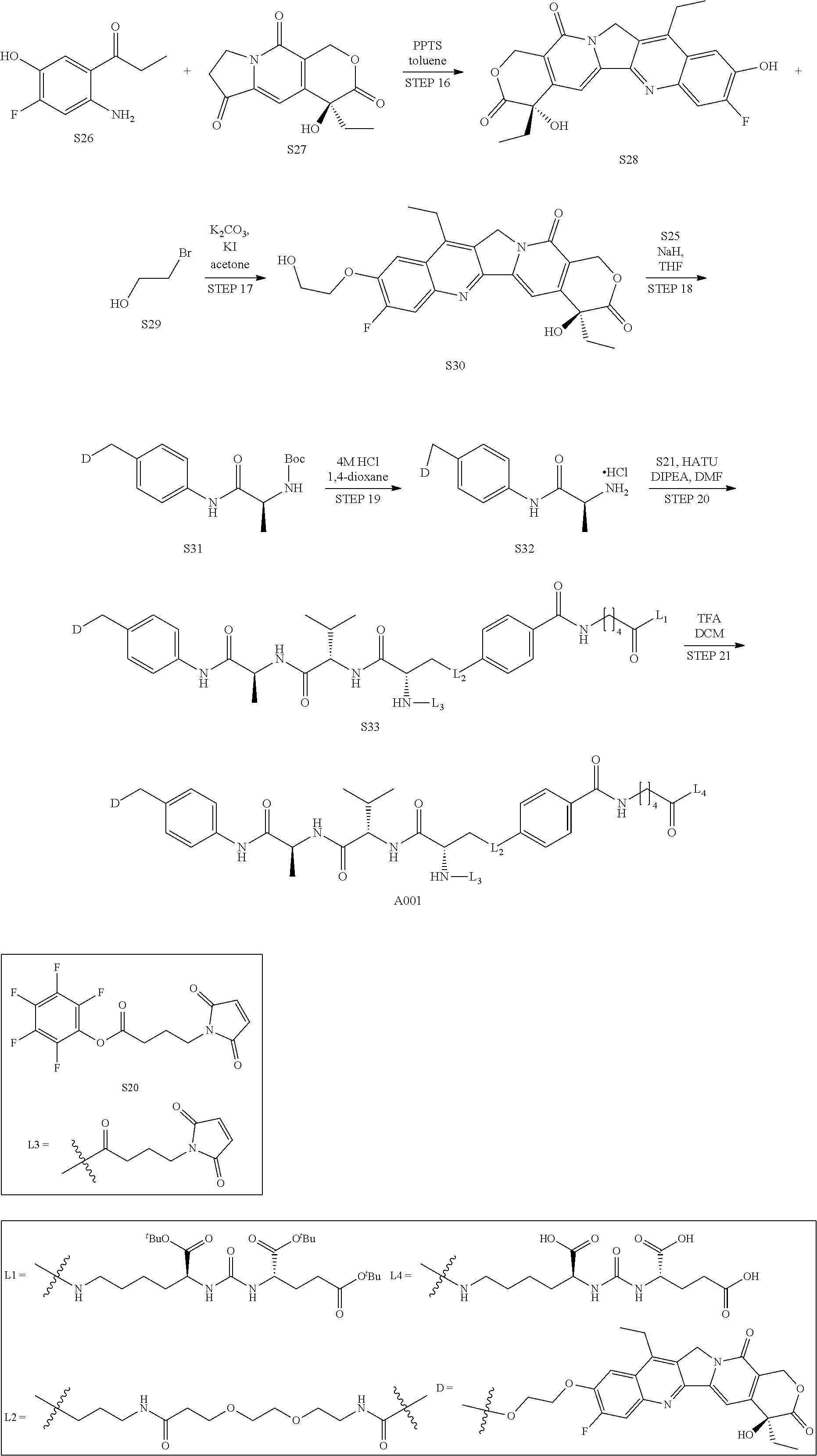

### Step 1:

To a stirring solution of triphosgene (33.43 g, 112.677 mmol) in CH₂Cl₂ (300 mL) at −78° C. were added a solution of S2 (100.00 g, 338.066 mmol) in CH₂Cl₂ (400 mL) and a solution of DIPEA (235.55 mL, 1352.265 mmol) in CH₂Cl₂ (100 mL) in sequence. The resulting mixture was stirred at −78° C. for 2 h before a mixture solution of S1 (126.06 g, 338.066 mmol) and DIPEA (58.89 mL, 338.066 mmol) in CH₂Cl₂ (400 mL) were added. The resulting mixture was naturally warmed to room temperature and stirred for 5 h before it was quenched with water (1 L). The layers were separated, and the aqueous layer was extracted with CH₂Cl₂ (3× 250 mL). The combined organic layers were dried (Na₂SO₄) and concentrated in vacuo. The intermediate S3 was obtained as a colorless oil (207.86 g, 99%) after flash column chromatography purification (silica gel, hexane:EtOAc=2:1). HRMS (ESI): calcd for C₃₂H₅₂N₃O₉⁺ [M+H]⁺ 622.3698, found 622.3704.

### Step 2:

To a stirring solution of S3 (311.80 g, 501.472 mmol) in MeOH (2 L) at room temperature was added Pd/C (31.20 g, 10% wt/wt). The resulting mixture was stirred at room temperature under H₂ atmosphere for 12 h before it was filtered through a pad of celite. The filtrate was collected and concentrated in vacuo. The intermediate S4 was obtained as a yellow oil (244.54 g, 100%) and used without further purification. HRMS (ESI): calcd for C₂₄H₄₆N₃O₇⁺ [M+H]⁺ 488.3330, found 488.3331.

### Step 3:

To a stirring solution of S4 (244.54 g, 501.472 mmol) and S₅ (189.02 g, 752.215 mmol) in CH₂Cl₂ (2.5 L) at 0° C. was added EDCI (144.20 g, 752.215 mmol). The resulting mixture was warmed to room temperature and stirred for 2 h before it quenched with citric acid (10% wt/wt, aq., 500 mL). The layers were separated, and the aqueous layer was washed with NaCl (sat. aq., 200 mL). The combined organic layers were dried (Na₂SO₄) and concentrated in vacuo. The intermediate S5 was obtained as a white solid (240.20 g, 66%) by recrystallization (CH₂Cl₂:hexane=1:1, room temperature). HRMS (ESI): calcd for C₃₇H₆₁N₄O₁₀⁺ [M+H]⁺ 721.4382, found 721.4385.

### Step 4:

To a stirring solution of S5 (80.27 g, 111.347 mmol) in MeOH (800 mL) at room temperature was added Pd/C (8.03 g, 10% wt/wt). The resulting mixture was stirred at room temperature under H₂ atmosphere for 3 h before it was filtered through a pad of celite. The filtrate was collected and concentrated in vacuo. The intermediate 5 was obtained as a yellow oil (65.34 g, 100%) and used without further purification. HRMS (ESI): calcd for C₂₉H₅₅N₄O₈⁺ [M+H]⁺ 587.4014, found 587.4022.

### Step 5:

To a stirring solution of S8 (100.00 g, 262.854 mmol) in CH₂Cl₂ (1.6 L) at 0° C. were added HATU (104.94 g, 275.996 mmol), S₉ (67.27 g, 275.996 mmol) and DIPEA (84.93 g, 657.134 mmol) in sequence. The resulting mixture was warmed to room temperature and stirred for 4 h before it was quenched with citric acid (10% wt/wt, aq., 500 mL). The layers were separated, and the aqueous layer was washed with NaCl (sat. aq., 200 mL). The combined organic layers were dried (Na₂SO₄) and concentrated in vacuo. The intermediate S10 was obtained as a white solid (135.00 g, 90%) by recrystallization (CH₂Cl₂:hexane=1:20, room temperature). HRMS (ESI): calcd for C₃₁H₄₄N₃O₇⁺ [M+H]⁺ 570.3174, found 570.3175.

### Step 6:

To a stirring solution of S10 (151.50 g, 265.929 mmol) in CH₂Cl₂ (1 L) at room temperature was added TFA (330 mL). The resulting mixture was stirred at room temperature for 2 h before it was concentrated in vacuo directly. The intermediate S11 was obtained as a yellow oil (124.88 g, 100%) and used without further purification. HRMS (ESI): calcd for C₂₆H₃₆N₃O₅⁺ [M+H]⁺ 470.2649, found 470.2653.

### Step 7:

To a stirring solution of S12 (73.75 g, 265.940 mmol) in THF (1 L) at 0° C. was added HATU (106.18 g, 279.2374 mmol). The resulting mixture was stirred at 0° C. for 20 min before addition of a solution of S11 (124.88 g, 265.929 mmol) in THF (500 mL) and DIPEA (103.12 g, 797.820 mmol) in sequence. The resulting mixture was warmed to room temperature and stirred for further 2 h before it was quenched with citric acid (10% wt/wt, aq., 1 L). The layers were separated, and the aqueous layer was extracted with CH₂Cl₂ (3× 300 mL). The combined organic layers were washed with NaCl (sat. aq., 500 mL), dried (Na₂SO₄) and concentrated in vacuo. The intermediate S13 was obtained as a yellow oil (193.84 g, 100%) after flash column chromatography (silica gel, CH₂Cl₂:hexane=10:1). HRMS (ESI): calcd for C₃₈H₅₇N₄O₁₀⁺ [M+H]⁺ 729.4069, found 729.4074.

### Step 8:

To a stirring solution of S13 (193.84 g, 265.929 mmol) in CH₂Cl₂ (1 L) at room temperature was added TFA (500 mL). The resulting mixture was stirred at room temperature for 2 h before it was concentrated in vacuo directly. The intermediate S14 was obtained as a yellow oil (167.22 g, 100%) and used without further purification. HRMS (ESI): calcd for C₃₃H₄₉N4O₈⁺ [M+H]⁺ 629.3545, found 629.3545.

### Step 9:

To a stirring solution of S14 (167.22 g, 265.948 mmol) in CH₂Cl₂ (1.5 L) at 0° C. were added S15 (59.10 g, 265.948 mmol) and EDCI (76.47 g, 398.922 mmol) in sequence. The resulting mixture was warmed to room temperature and stirred for 4 h before it quenched with citric acid (10% wt/wt, aq., 500 mL). The layers were separated, and the aqueous layer was washed with Na₂CO₃ (5% wt/wt, aq., 200 mL). The combined organic layers were dried (Na₂SO₄) and concentrated in vacuo. The intermediate S16 was obtained as a yellow oil (221.53, 100%) and used without further purification. HRMS (ESI): calcd for C₄₅H₆₁N₄O₁₁⁺ [M+H]⁺ 833.4331, found 833.4333.

### Step 10:

To a stirring solution of S16 (221.53 g, 265.948 mmol) in CH₂Cl₂ (600 mL) at 40° C. was added HCO₂H (1.2 L). The resulting mixture was stirred at 40° C. for 12 h before it was concentrated in vacuo directly. The residue was dissolved with water (1 L) and extracted with EtOAc (1 L×3). The combined organic layers were dried (Na₂SO₄) and concentrated in vacuo. The intermediate S17 was obtained as a white solid (162.00 g, 78% over 5 steps) by recrystallization (hexane:EtOAc=1:1, room temperature). HRMS (ESI): calcd for C₄₁H₅₃N₄O₁₁⁺ [M+H]⁺ 777.3705, found 777.3706.

### Step 11:

To a stirring solution of S17 (49.20 g, 63.330 mmol) in CH₂Cl₂ (500 mL) at room temperature were added HATU (28.90 g, 75.996 mmol) and DIPEA (12.28 g, 94.995 mmol) in sequence. The resulting mixture was stirred at room temperature for 0.5 h before addition of a solution of 7 (37.16 g, 63.330 mmol) in CH₂Cl₂ (200 mL). The resulting mixture was stirred at room temperature for further 1 h before it was quenched with HCl (1 M aq, 200 mL). The layers were separated and the aqueous layer was extracted with CH₂Cl₂ (3× 100 mL). The combined organic layers were washed with NaCl (sat. aq., 500 mL), dried (Na₂SO₄) and concentrated in vacuo. The intermediate S18 was obtained as a yellow oil (75.23 g, 88%) after flash column chromatography purification (silica gel, CH₂Cl₂:hexane=10:1). HRMS (ESI): calcd for C₇₀H₁₀₅N₈O₁₈⁺ [M+H]⁺ 1345.7541, found 1345.7543.

### Step 12:

To a stirring solution of S18 (100.00 g, 74.314 mmol) in MeOH (1 L) at room temperature was added Pd/C (10.00 g, 10% wt/wt). The resulting mixture was stirred at room temperature under H₂ atmosphere for 12 h before it was filtered through a pad of celite. The filtrate was collected and concentrated in vacuo. The intermediate S19 was obtained as a yellow solid (79.89 g, 96%) after trituration with EtOAc (500 mL). HRMS (ESI): calcd for C₅₅H₉₃N₈O₁₆⁺ [M+H]⁺ 1121.6704, found 1121.6704.

### Step 13:

To a stirring solution of S19 (208.10 g, 185.575 mmol) in DMF (3 L) at room temperature were added S20 (77.77 g, 222.690 mmol) and DIPEA (59.96 g, 463.937 mmol). The resulting mixture was stirred at room temperature for 3 h before it was concentrated in vacuo directly. The intermediate S21 was obtained as a light yellow solid (165.00 g, 69%) after flash column chromatography purification (silica gel, CH₂Cl₂:hexane=10:1). HRMS (ESI): calcd for C₆₃H₁₀₀N₉O₁₉⁺ [M+H]⁺ 1286.7130, found 1286.7136.

### Step 14:

To a stirring solution of S22 (22.00 g, 178.629 mmol) and S₂₃ (33.80 g, 178.629 mmol) in THF (200 mL) at room temperature was added EEDQ (48.59 g, 196.492 mmol). The resulting mixture was stirred at room temperature for 15 h before it was concentrated in vacuo directly. The intermediate S24 was obtained as a light yellow solid (39.13 g, 74%) after trituration with hexane/EtOAc (50% V/V). HRMS (ESI): calcd for C₁₅H₂₃N2O₄⁺ [M+H]⁺ 295.1652, found 295.1654.

### Step 15:

To a stirring solution of S24 (20 g, 67.946 mmol) in THF (300 mL) at 0° C. were added PPh₃ (35.64 g, 135.893 mmol) and NBS (24.19 g, 135.893 mmol) in sequence. The resulting mixture was stirred at 0° C. for 1 h before it was quenched with water (100 mL). The layers were separated and the aqueous layer was extracted with EtOAc (3×50 mL). The combined organic layers were dried (Na₂SO₄) and concentrated in vacuo. The intermediate S25 was obtained as a white solid (17.23 g, 72%) after flash column chromatography purification (silica gel, hexane:EtOAc=2:1). HRMS (ESI): calcd for C₁₅H₂₂BrN₂O₃⁺ [M+H]⁺ 357.0808, found 357.0806.

### Step 16:

To a stirring solution of S26 (30 g, 163.773 mmol) and S₂₇ (43 g, 163.773 mmol) in toluene (600 mL) at room temperature was added PPTS (2.06 g, 8.189 mmol). The resulting mixture was heated to reflux and stirred overnight before it was cooled to room temperature and filtered. The filter cake was collected and stirred in toluene at reflux for 2 h before it was cooled and filtered. The filter cake was collected and dried in vacuo. The intermediate S28 was obtained as a dark yellow solid (61 g, 91%). HRMS (ESI): calcd for C₂₂H₂₀FN₂O₅⁺ [M+H]⁺ 411.1351, found 411.1355.

### Step 17:

To a stirring solution of S28 (4.14 g, 10.088 mmol) and S₂₉ (7.56 g, 60.526 mmol) in acetone (150 mL) at room temperature were added K₂CO₃ (4.18 g, 30.263 mmol) and KI (1.51 g, 10.088 mmol) in sequence. The resulting mixture was heated to 60° C. and stirred for 20 h before it was cooled to room temperature and concentrated in vacuo directly. The intermediate S30 was obtained as a dark yellow solid (2.6 g, 57%) after flash column chromatography purification (silica gel, DCM:MeOH=10:1). HRMS (ESI): calcd for C₂₄H₂₄FN₂O₆⁺ [M+H]⁺ 455.1613, found 455.1614.

### Step 18:

To a stirring solution of S30 (2.00 g, 4.401 mmol) in THF (50 mL) at 0° C. was added NaH (264 mg, 6.601 mmol, 60% dispersion in mineral oil). The resulting mixture was stirred at 0° C. for 0.5 h before addition of S₂₅ (1.57 g, 4.401 mmol). The resulting mixture was stirred at 0° C. for 1 h before it was quenched with NH₄Cl (sat. aq., 30 mL). The layers were separated, and the aqueous layer was extracted with EtOAc (3×30 mL). The combined organic layers were dried (Na₂SO₄) and concentrated in vacuo. The intermediate S31 was prepared after RP-HPLC purification and lyophilization as a yellow solid (2.88 g, 89%). HRMS (ESI): calcd for C₃₉H₄₄FN₄O₉⁺ [M+H]⁺ 731.3087, found 731.3090.

### Step 19:

To a stirring solution of S31 (2.00 g, 2.737 mmol) in 1,4-dioxane (20 mL) at 0° C. was added HCl (3.42 mL, 13.684 mmol, 4.0 M in 1,4-dioxane). The resulting mixture was stirred at 0° C. for 1 h before it was concentrated in vacuo directly. The intermediate S32 was obtained as a yellow oil (1.92 g, 105%) and used without further purification. HRMS (ESI): calcd for C₃₄H₃₆FN₄O₇⁺ [M+H]⁺ 631.2563, found 631.2565.

### Step 20:

To a stirring solution of S21 (4.22 g, 3.284 mmol) in DMF (50 mL) at room temperature was added HATU (1.25 g, 3.284 mmol). The resulting mixture was stirred at room temperature for 15 min before addition of S₃₂ (1.92 g, 2.737 mmol) and DIPEA (1.06 g, 3.284 mmol) in sequence. The resulting mixture was stirred at room temperature for 30 min before it was concentrated in vacuo directly. The intermediate S33 was obtained as a yellow oil (8.08 g, 284%) and used without further purification. HRMS (ESI): calcd for C₉₇H₁₃₃FN₁₃O₂₅⁺ [M+H]⁺ 1898.9514, found 1898.9514.

### Step 21:

To a stirring solution of S32 (8.08 g, 2.737 mmol) in CH₂Cl₂ (50 mL) at room temperature was added TFA (50 mL). The resulting mixture was stirred at room temperature for 4 h before it was concentrated in vacuo. The product A001 was obtained as a yellow solid (1.02 g, 21% over 3 steps) after RP-HPLC purification and lyophilization. HRMS (ESI): calcd for C₈₅H₁₀₉FN₁₃O₂₅⁺ [M+H]⁺ 1730.7636, found 1730.7636.

### Example 351: Synthesis of A002

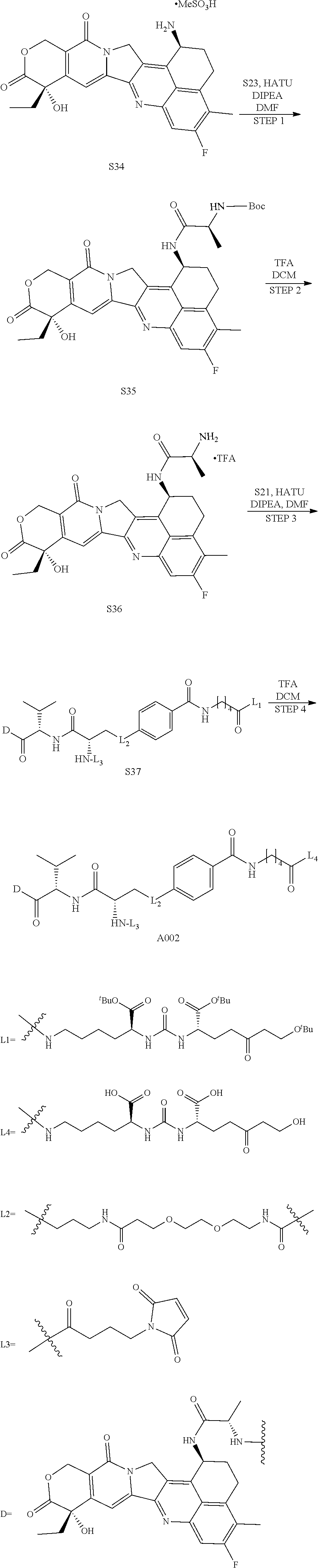

### Step 1:

To a stirring solution of S23 (7.84 g, 41.420 mmol) in DMF (400 mL) at room temperature were added HATU (15.75 g, 41.420 mmol) and DIPEA (12.17 g, 94.135 mmol) in sequence. The resulting mixture was stirred at room temperature for 30 min before addition of S₃₄ (20.00 g, 37.654 mmol). The resulting mixture was stirred at room temperature for further 1 h before it was quenched with EtOAc (2 L) and filtered. The filter cake was collected and dried in vacuo. The intermediate S35 was obtained as a yellow solid (22.80 g, 99%) and used without further purification. HRMS (ESI): calcd for C₃₂H₃₆FN₄O₇⁺ [M+H]⁺ 607.2563, found 607.2568.

### Step 2:

To a stirring solution of S35 (10.00 g, 16.484 mmol) in CH₂Cl₂ (150 mL) at room temperature was added TFA (75 mL). The resulting mixture was stirred at room temperature for 2 h before it was concentrated in vacuo directly. The intermediate S36 was obtained as a yellow solid (9.22 g, 91%) after trituration with MTBE (1 L). HRMS (ESI): calcd for C₂₇H₂₈FN₄O₅⁺ [M+H]⁺ 507.2038, found 507.2044.

### Step 3:

To a stirring solution of S21 (8.18 g, 6.358 mmol) in DMF (150 mL) at room temperature was added HATU (2.54 g, 6.676 mmol). The resulting mixture was stirred at room temperature for 10 min before addition of S₃₆ (4.14 g, 6.676 mmol) and DIPEA (2.05 g, 15.895 mmol) in sequence. The resulting mixture was stirred at room temperature for further 1 h before it was concentrated in vacuo directly. The intermediate S37 was obtained as a dark yellow oil (11.29 g, 100%) and used without further purification. HRMS (ESI): calcd for C₉₀H₁₂₅FN₁₃O₂₃⁺ [M+H]⁺ 1774.8990, found 1774.8990.

### Step 4:

To a stirring solution of S37 (11.29 g, 6.360 mmol) in CH₂Cl₂ (50 mL) at room temperature was added TFA (100 mL). The resulting mixture was stirred at room temperature for 2 h before it was concentrated in vacuo directly. The product A002 was obtained as a yellow solid (5.68 g, 55% over 3 steps) after RP-HPLC purification and lyophilization. HRMS (ESI): calcd for C₇₈H₁₀₁FN₁₃O₂₃⁺ [M+H]⁺ 1606.7112, found 1606.7113.

### Example 352: Synthesis of A003

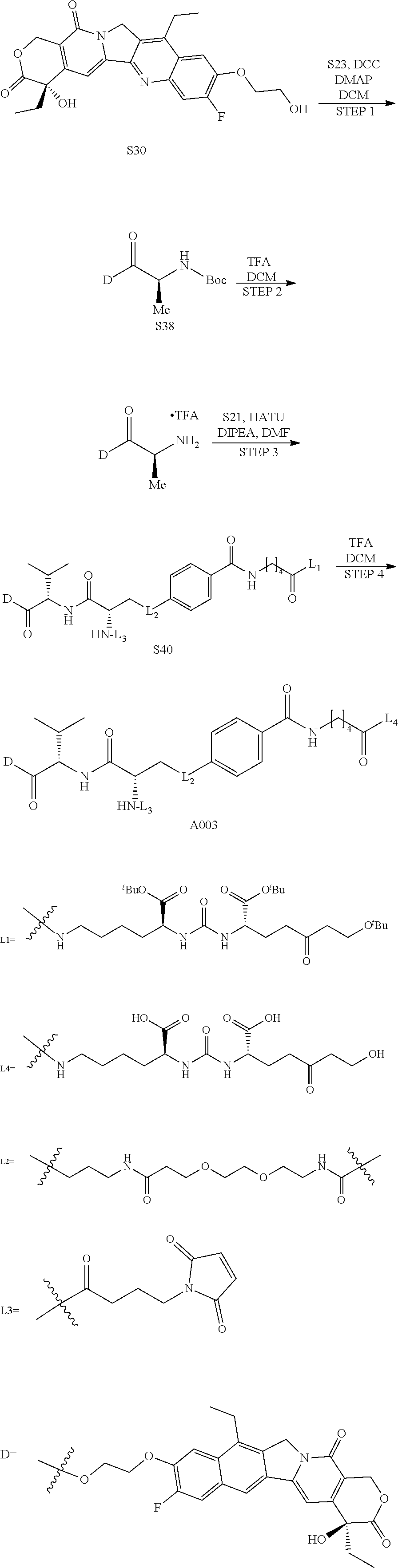

### Step 1:

To a stirring solution of S30 (700 mg, 1.540 mmol) and S₂₃ (292 mg, 1.540 mmol) in CH₂Cl₂ (10 mL) at were room temperature added DCC (636 mg, 3.081 mmol) and DMAP (188.18 mg, 1.540 mmol) in sequence. The resulting mixture was stirred at room temperature for 1 h before it was concentrated in vacuo directly. The intermediate S38 was obtained as a yellow solid (840 mg, 87%) after flash column chromatography purification (silica gel, CH₂Cl₂:MeOH=10:1). HRMS (ESI): calcd for C₃₂H₃₇FN₃O₉⁺ [M+H]⁺ 626.2508, found 626.2516.

To a stirring solution of S38 (150 mg, 0.240 mmol) in CH₂Cl₂ (4 mL) at room temperature was added TFA (2 mL). The resulting mixture was stirred at room temperature for 0.5 h before it was concentrated in vacuo directly. The intermediate S39 was obtained as a yellow oil (153 mg, 100%) and used without further purification. HRMS (ESI): calcd for C₂₇H₂₉FN₃O₇⁺ [M+H]⁺ 526.1984, found 526.1983.

### Step 3:

To a stirring solution of S21 (280 mg, 0.218 mmol) in DMF (3 mL) at room temperature was added HATU (87 mg, 0.229 mmol). The resulting mixture was stirred at room temperature for 10 min before addition of S₃₉ (153 mg, 0.240 mmol) and DIPEA (70 mg, 0.545 mmol) in sequence. The resulting mixture was stirred at room temperature for further 1 h before it was concentrated in vacuo directly. The intermediate S40 was obtained as a yellow oil (391 mg, 100%) and used without further purification. HRMS (ESI): calcd for C₉₀H₁₂₆FN₁₂O₂₅⁺ [M+H]⁺ 1793.8936, found 1794.2627.

### Step 4:

To a stirring solution of S40 (391 mg, 0.218 mmol) in CH₂Cl₂ (3 mL) at room temperature was added TFA (3 mL). The resulting mixture was stirred at room temperature for 1 h before it was concentrated in vacuo directly. The product A003 was obtained as a yellow solid (38.0 mg, 10% over 3 steps) after RP-HPLC purification and lyophilization. HRMS (ESI): calcd for C₇₈H₁₀₂FN₁₂O₂₅⁺ [M+H]⁺ 1625.7058, found 1625.7060.

### Example 353: Synthesis of A004

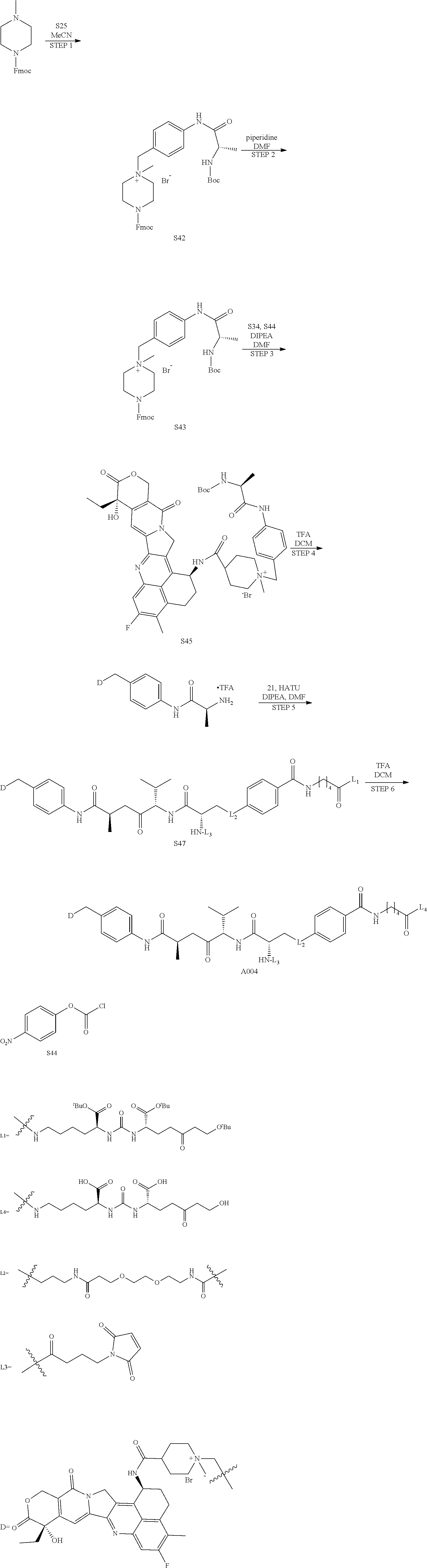

### Step 1:

To a stirring solution of S41 (3.00 g, 9.305 mmol) in MeCN (30 mL) at room temperature was added S25 (3.32 g, 9.305 mmol). The resulting mixture was stirred at room temperature for 24 h before it was quenched with henane/EtOAc (1:1) and filtered. The filter cake was collected and dried in vacuo. The intermediate S42 was obtained as a yellow solid (4.20 g, 75%) and used without further purification. HRMS (ESI): calcd for C₃₅H₄₃N4O₅⁺ M⁺ 599.3228, found 599.3226.

### Step 2:

To a stirring solution of S42 (4.10 g, 6.836 mmol) in DMF (30 mL) at room temperature was added piperidine (3 mL). The resulting mixture was stirred at room temperature for 15 min before it was concentrated in vacuo directly. The intermediate S43 was obtained as a white solid (2.58 g, 100%) after trituration with hexane (100 mL). HRMS (ESI): calcd for C₂₀H₃₃N4O₃⁺ M⁺ 377.2547, found 377.2548.

### Step 3:

To a stirring solution of S34 (500 mg, 0.941 mmol) in DMF (10 mL) at 0° C. were added S44 (190 mg, 0.941 mmol) and DIPEA (486 mg, 0.941 mmol) in sequence. The resulting mixture was stirred at 0° C. for 30 min before addition of S₄₃ (355 mg, 0.941 mmol). The resulting mixture was stirred at 0° C. for 1 h before it was quenched with EtOAc (50 mL) and filtered. The filter cake was collected and dried in vacuo. The intermediate S45 was obtained as a yellow solid (790 mg, 100%) without further purification. HRMS (ESI): calcd for C₄₅H₅₃FN₇O₈⁺ M⁺ 838.3934, found 838.3942.

### Step 4:

To a stirring solution of S45 (790 mg, 0.941 mmol) in CH₂Cl₂ (6 mL) at room temperature was added TFA (3 mL). The resulting mixture was stirred at room temperature for 1 h before it was concentrated in vacuo directly. The intermediate S46 was obtained as a yellow oil (787 mg, 100%) and used without further purification. HRMS (ESI): calcd for C₄₀H₄₅FN₇O₆⁺ M⁺ 738.3410, found 738.3416.

### Step 5:

To a stirring solution of S21 (1.00 g, 0.784 mmol) in DMF (15 mL) at room temperature was added HATU (358 mg, 0.941 mmol). The resulting mixture was stirred at room temperature for 15 min before addition of S₄₆ (787 mg, 0.941 mmol) and DIPEA (304 mg, 2.353 mmol) in sequence. The resulting mixture was stirred at room temperature for further 1 h before it was concentrated in vacuo directly. The intermediate S47 was obtained as a dark yellow oil (1.57 g, 100%) and used without further purification. HRMS (ESI): calcd for C₁₀₃H₁₄₂FN₁₆O₂₄⁺ [M+H]⁺ 2006.0361, found 2006.0364.

### Step 6:

To a stirring solution of S47 (1.57 g, 0.784 mmol) in CH₂Cl₂ (20 mL) at room temperature was added TFA (10 mL). The resulting mixture was stirred at room temperature for 1 h before it was concentrated in vacuo directly. The product A004 was obtained as a yellow solid (350 mg, 20% over 4 steps) after RP-HPLC purification and lyophilization. HRMS (ESI): calcd for C₉₁H₁₁₈FN₁₆O₂₄⁺ [M+H]⁺ 1837.8483, found 1837.8482.

### Example 354: Synthesis of A005

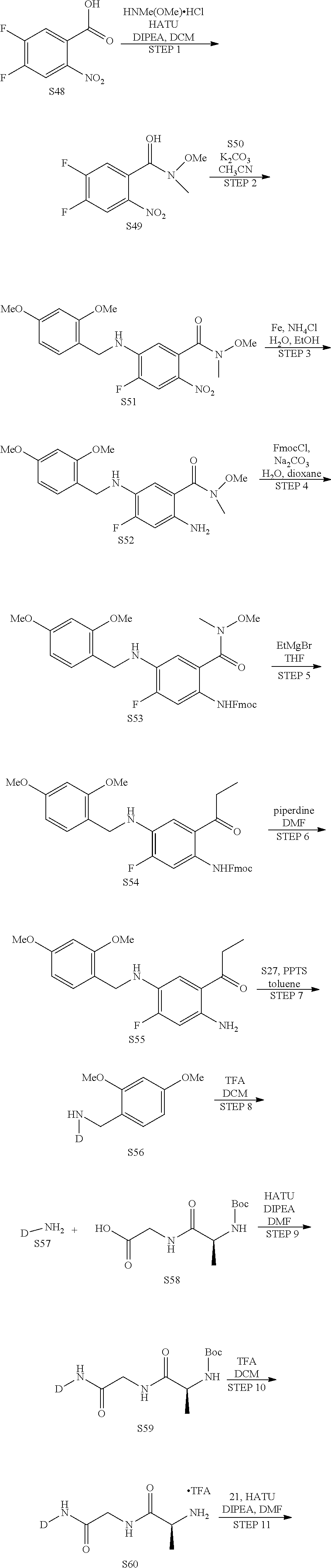

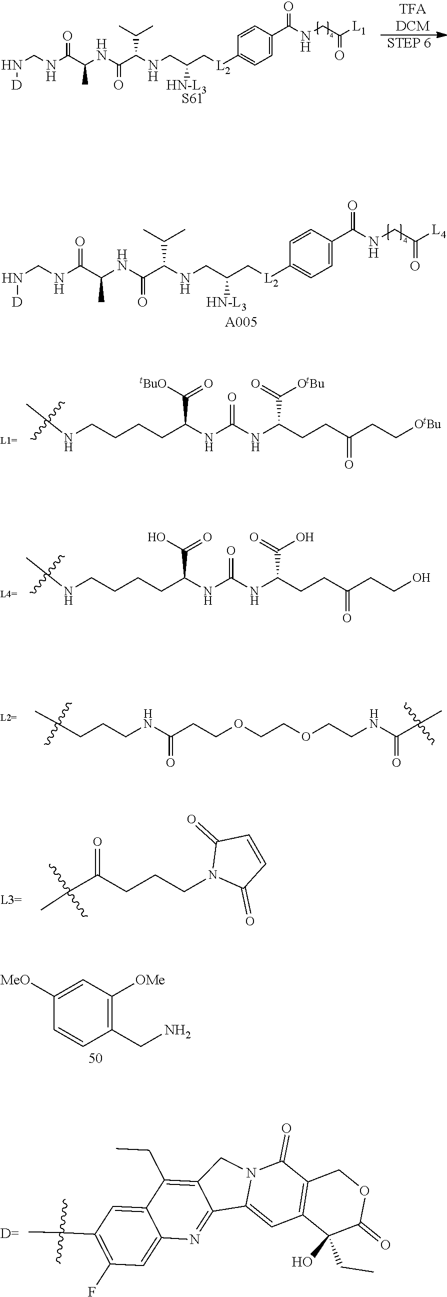

### Step 1:

To a stirring solution of S48 (2.00 g, 9.847 mmol) and HNMe (OMe)·HCl (1.15 g, 11.817 mmol) in CH₂Cl₂ (20 mL) at room temperature were added HATU (4.49 g, 11.817 mmol) and DIPEA (3.82 g, 29.541 mmol). The resulting mixture was stirred at room temperature for 1 h before it was quenched with Na₂CO₃ (5% wt/wt aq., 10 mL). The layers were separated, and the organic layer was washed with HCl (1 M aq., 10 mL) and water (10 mL). The organic layers were dried (Na₂SO₄) and concentrated in vacuo. The intermediate S49 was obtained as a yellow solid (2.42 g, 100%) and used without further purification. HRMS (ESI): calcd for C₉H₉F₂N₂O₄⁺ [M+H]⁺ 247.0525, found 247.0528.

To a stirring solution of S49 (2.42 g, 9.847 mmol) and S₅₀ (2.47 g, 14.746 mmol) in MeCN (20 mL) at room temperature was added K₂CO₃ (4.08 g, 29.492 mmol). The resulting mixture was heated to 80° C. and stirred for 2 h before it was cooled to room temperature and quenched with water (50 mL). The layers were separated, and the aqueous layer was extracted with EtOAc (50 mL×3). The combined organic layers were dried (Na₂SO₄) and concentrated in vacuo. The intermediate S51 was obtained as a yellow solid (3.87 g, 100%) and used without further purification. HRMS (ESI): calcd for C₁₈H₂₁FN₃O₆⁺ [M+H]⁺ 394.1409, found 394.1414.

### Step 3:

To a stirring solution of S51 (3.87 g, 9.847 mmol) in EtOH/H₂O (30 mL, 50% V/V) at room temperature were added NH₄Cl (5.26 g, 98.381 mmol) and Fe powder (3.85 g, 68.866 mmol). The resulting mixture was heated to 80° C. and stirred for 2 h before it was cooled to room temperature and filtered through a pad of celite. The filtrate was collected and concentrated in vacuo. The intermediate S₅₂ was obtained as a yellow solid (3.58 g, 100%) and used without further purification. HRMS (ESI): calcd for C₁₈H₂₃FN₃O₄⁺ [M+H]⁺ 364.1667, found 364.1672.

### Step 4:

To a stirring solution of S52 (3.58 g, 9.847 mmol) in 1,4-dioxane/H₂O (100 mL, 70% V/V) at 0° C. were added Na₂CO₃ (2.09 g, 19.694 mmol) and FmocCl (6.37 g, 24.618 mmol). The resulting mixture was warmed to room temperature and stirred for 12 h before it was concentrated in vacuo. The residual aqueous layer was extracted with EtOAc (50 mL×3). The combined organic layers were dried (Na₂SO₄) and concentrated in vacuo. The intermediate S53 was obtained as a white solid (5.00 g, 87% over 4 steps) after flash column chromatography purification (silica gel, hexane:EtOAc=3:1). HRMS (ESI): calcd for C₃₃H₃₃FN₃O₆⁺ [M+H]⁺ 586.2348, found 586.2351.

### Step 5:

To a stirring solution of S53 (53.00 g, 65.604 mmol) in THF (500 mL) at 0° C. was added EtMgBr (154.4 mL, 3.4 mol/L in 2-Me-THF, 524.831 mmol). The resulting mixture was warmed to room temperature and stirred for 6 h before it was quenched with NH₄Cl (sat. aq., 800 mL). The layers were separated, and the aqueous layer was extracted with EtOAc (500 mL×3). The combined organic layers were dried (Na₂SO₄) and concentrated in vacuo. The intermediate S54 was obtained as a yellow oil (50.19 g, 100%) and used without further purification. HRMS (ESI): calcd for C₃₃H₃₂FN₂O₅⁺ [M+H]⁺ 555.2290, found 555.2298.

### Step 6:

To a stirring solution of S54 (50.19 g, 65.604 mmol) in DMF (500 mL) at room temperature was added piperidine (50 mL). The resulting mixture was stirred at room temperature for 30 min before it was concentrated in vacuo directly. The intermediate S55 was obtained as a yellow solid (21.48 g, 71% over 2 steps) flash column chromatography (silica gel, hexane:EtOAc=8:1). HRMS (ESI): calcd for C₁₈H₂₂FN₂O₃⁺ [M+H]⁺ 333.1609, found 333.1618.

### Step 7:

To a stirring solution of S55 (25.30 g, 76.118 mmol) and S₂₇ (20.04 g, 76.118 mmol) in toluene (500 mL) at room temperature was added PPTS (1.91 g, 7.612 mmol). The resulting mixture was heated to reflux and stirred overnight before it was cooled to room temperature and filtered. The filter cake was collected and stirred in refluxing toluene for 2 h before it was cooled and filtered. The filter cake was collected and dried in vacuo. The intermediate S56 was obtained as a dark yellow solid (35.54 g, 88%). HRMS (ESI): calcd for C₃₁H₃₁FN₃O₆⁺ [M+H]⁺ 560.2191, found 560.2200.

### Step 8:

To a stirring solution of S56 (21 g, 37.527 mmol) in CH₂Cl₂ (40 mL) at room temperature was added TFA (20 mL). The resulting mixture was stirred at room temperature for 1 h before it was concentrated in vacuo directly. The intermediate S57 was obtained as a yellow solid (10.20 g, 66%) after flash column chromatography purification (silica gel, CH₂Cl₂:hexane=20:1). HRMS (ESI): calcd for C₂₂H₂₁FN₃O₄⁺ [M+H]⁺ 410.1511, found 410.1512.

### Step 9:

To a stirring solution of S58 (1.00 g, 4.061 mmol) in DMF (20 mL) at room temperature were added HATU (1.70 g, 4.467 mmol) and DIPEA (1.57 g, 12.183 mmol) in sequence. The resulting mixture was stirred at room temperature for 15 min before addition of 57 (1.83 g, 4.467 mmol). The resulting mixture was stirred at room temperature for further 1 h before it was concentrated in vacuo directly. The intermediate S59 was obtained as a dark yellow oil (1.31 g, 92%) after flash column chromatography purification (silica gel, CH₂Cl₂:hexane=20:1). HRMS (ESI): calcd for C₃₂H₃₇FN₅O₈⁺ [M+H]⁺ 638.2621, found 638.2626.

### Step 10:

To a stirring solution of S59 (1.31 g, 2.054 mmol) in CH₂Cl₂ (10 mL) at room temperature was added TFA (5 mL). The resulting mixture was stirred at room temperature for 1 h before it was concentrated in vacuo directly. The residue was dispersed with MTBE (50 mL) and stirred for 30 min before it was filtered. The filter cake was collected and dried in vacuo. The intermediate S60 was obtained as a yellow solid (1.31 g, 100%) and used without further purification. HRMS (ESI): calcd for C₂₇H₂₉FN₅O₆⁺ [M+H]⁺ 538.2096, found 538.2100.

### Step 11:

To a stirring solution of S21 (2.64 g, 2.054 mmol) in DMF (50 mL) at room temperature was added HATU (781 mg, 2.054 mmol). The resulting mixture was stirred at room temperature for 15 min before addition of S₆₀ (1.31 g, 2.054 mmol) and DIPEA (797 mg, 6.162 mmol) in sequence. The resulting mixture was stirred at room temperature for further 1 h before it was concentrated in vacuo directly. The intermediate S61 was obtained as a dark yellow oil (3.71 g, 100%) and used without further purification. HRMS (ESI): calcd for C₉₀H₁₂₆FN₁₄O₂₄⁺ [M+H]⁺ 1805.9048, found 1805.9057.

### Step 12:

To a stirring solution of S61 (3.71 g, 2.054 mmol) in CH₂Cl₂ (50 mL) at room temperature was added TFA (25 mL). The resulting mixture was stirred at room temperature for 1 h before it was concentrated in vacuo directly. The product A005 was obtained as a yellow solid (1.51 g, 45% over 3 steps) after RP-HPLC purification and lyophilization. HRMS (ESI): calcd for C₇₈H₁₀₂FN₁₄O₂₄⁺ [M+H]⁺ 1637.7170, found 1637.7172.

### Example 355: Synthesis of A006

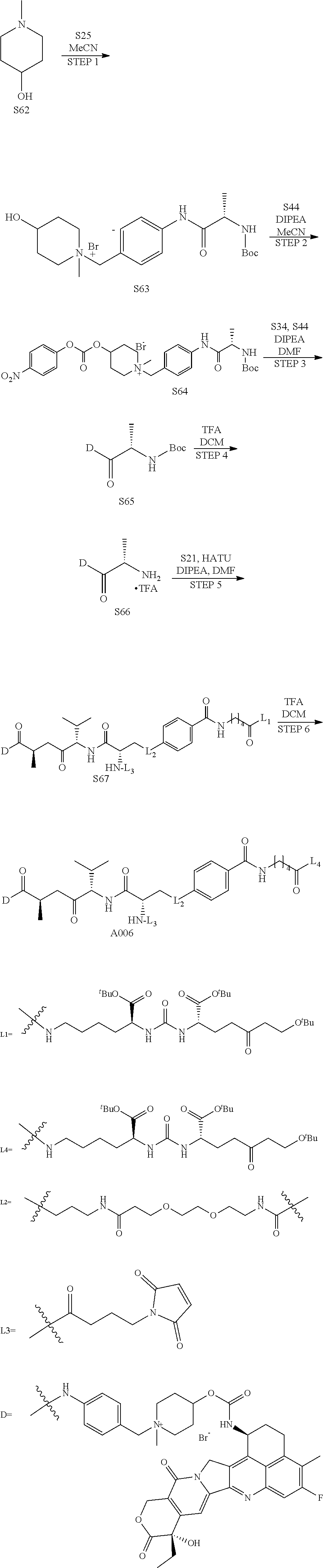

### Step 1:

To a stirring solution of S62 (300 mg, 2.605 mmol) in MeCN (10 mL) at room temperature was added S25 (930 mg, 2.605 mmol). The resulting mixture was stirred at room temperature for 12 h before it was concentrated in vacuo directly. The residue was dispersed with MTBE (50 mL) and stirred for 30 min before it was filtered. The filter cake was collected and dried in vacuo. The intermediate S63 was obtained as a white solid (1.19 g, 97%) and used without further purification. HRMS (ESI): calcd for C₂₁H₃₄N3O₄⁺ M⁺ 392.2544, found 392.2547.

To a stirring solution of S63 (1.19 g, 2.514 mmol) in MeCN (30 mL) at 0° C. were added S44 (608 mg, 3.016 mmol) and DIPEA (975 mg, 7.541 mmol) in sequence. The resulting mixture was stirred at 0° C. for 2 h before it was concentrated in vacuo directly. The residue was dispersed with MTBE (50 mL) and stirred for 30 min before it was filtered. The filter cake was collected and dried in vacuo The intermediate S64 was obtained as a white solid (1.41 g, 90%) and used without further purification. HRMS (ESI): calcd for C₂₈H₃₇N4O₇⁺ M⁺ 541.2657, found 541.2660.

### Step 3:

To a stirring solution of S64 (1.41 g, 2.265 mmol) in DMF (30 mL) at room temperature were added S34 (1.20 g, 2.265 mmol) and DIPEA (879 mg, 6.795 mmol) in sequence. The resulting mixture was stirred at room temperature for 2 h before it was concentrated in vacuo directly. The residue was dispersed with MTBE (50 mL) and stirred for 30 min before it was filtered. The intermediate S65 was obtained as a yellow solid (2.11 g, 100%) and used without further purification. HRMS (ESI): calcd for C₄₆H₅₄FN₆O₉⁺ M⁺ 853.3931, found 853.3935.

### Step 4:

To a stirring solution of S65 (2.11 g, 2.259 mmol) in CH₂Cl₂ (50 mL) at room temperature was added TFA (25 mL). The resulting mixture was stirred at room temperature for 1 h before it was concentrated in vacuo directly. The residue was dispersed with MTBE (50 mL) and stirred for 30 min before it was filtered. The filter cake was collected and dried in vacuo The intermediate S66 was obtained as a yellow solid (2.10 g, 99%) and used without further purification. HRMS (ESI): calcd for C₄₁H₄₆FN₆O₇⁺ M⁺ 753.3407, found 753.3407.

### Step 5:

To a stirring solution of S21 (2.90 g, 2.256 mmol) in DMF (50 mL) at room temperature was added HATU (858 mg, 2.256 mmol). The resulting mixture was stirred at room temperature for 15 min before addition of S₆₆ (2.10 g, 2.256 mmol) and DIPEA (875 mg, 6.768 mmol) in sequence. The resulting mixture was stirred at room temperature for further 1 h before it was concentrated in vacuo directly. The intermediate S67 was obtained as a dark yellow oil (4.74 g, 100%) and used without further purification. HRMS (ESI): calcd for C₁₀₄H₁₄₃FN₁₅O₂₅⁺ M⁺ 2021.0358, found 2021.0360.

### Step 6:

To a stirring solution of S67 (4.74 g, 2.256 mmol) in CH₂Cl₂ (50 mL) at room temperature was added TFA (25 mL). The resulting mixture was stirred at room temperature for 1 h before it was concentrated in vacuo directly. The product A006 was obtained as a yellow solid (1.95 g, 46% over 2 steps) after RP-HPLC purification and lyophilization. HRMS (ESI): calcd for C₉₂H₁₁₉FN₁₅O₂₅⁺ [M+H] 1852.8480, found 1852.8484.

### Example 356: Synthesis of A007

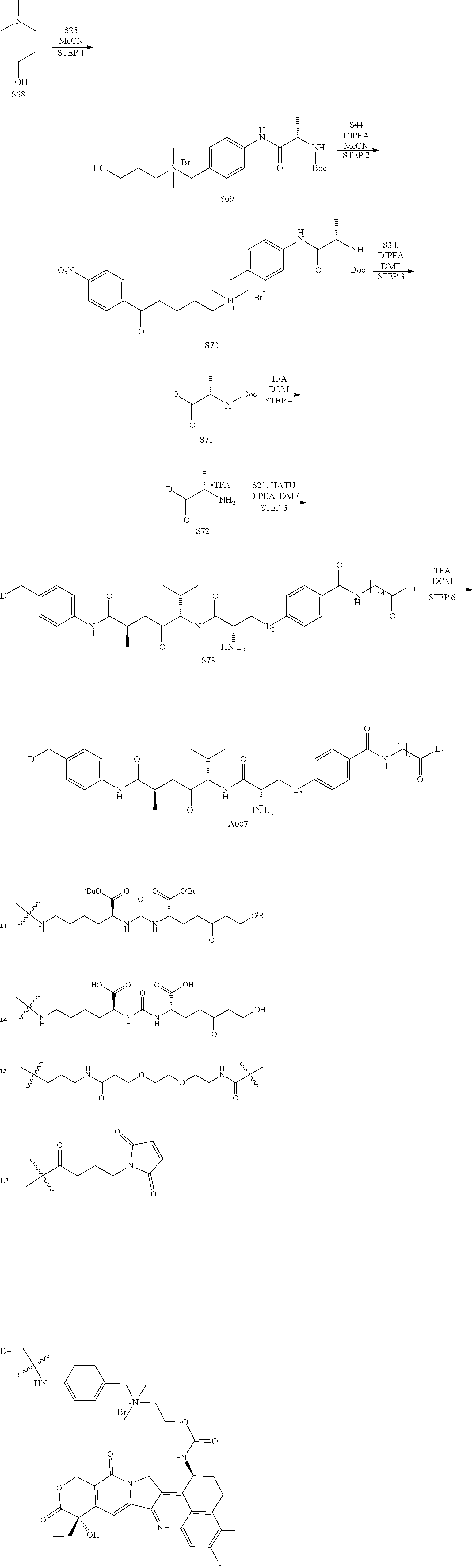

### Step 1:

To a stirring solution of S68 (300 mg, 2.908 mmol) in MeCN (10 mL) at room temperature was added S25 (1.04 g, 2.908 mmol). The resulting mixture was stirred at room temperature for 12 h before it was concentrated in vacuo directly. The residue was dispersed with MTBE (50 mL) and stirred for 30 min before it was filtered. The filtrate was collected and concentrated in vacuo. The intermediate S69 was obtained as a white solid (1.33 g, 99%) and used without further purification. HRMS (ESI): calcd for C₂₀H₃₄N3O₄⁺ M⁺ 380.2544, found 380.2549.

### Step 2:

To a stirring solution of S69 (1.33 g, 2.889 mmol) in MeCN (30 mL) at 0° C. were added S44 (699 mg, 3.466 mmol) and DIPEA (1.17 g, 8.666 mmol) in sequence. The resulting mixture was stirred at 0° C. for 2 h before it was concentrated in vacuo directly. The residue was dispersed with MTBE (50 mL) and stirred for 30 min before it was filtered. The filter cake was collected and dried in vacuo The intermediate S70 was obtained as a white solid (1.68 g, 95%) and used without further purification. HRMS (ESI): calcd for C₂₇H₃₇N4O₇⁺ M⁺ 529.2657, found 529.2658.

### Step 3:

To a stirring solution of S70 (1.68 g, 2.756 mmol) in DMF (30 mL) at room temperature were added S34 (1.46 g, 2.756 mmol) and DIPEA (1.07 g, 8.269 mmol) in sequence. The resulting mixture was stirred at room temperature for 2 h before it was concentrated in vacuo directly. The residue was dispersed with MTBE (50 mL) and stirred for 30 min before it was filtered. The intermediate S71 was obtained as a yellow solid (2.20 g, 88%) and used without further purification. HRMS (ESI): calcd for C₄₄H₅₂FN₆O₉⁺ M⁺ 827.3774, found 827.3778.

### Step 4:

To a stirring solution of S71 (2.20 g, 2.423 mmol) in CH₂Cl₂ (40 mL) at room temperature was added TFA (20 mL). The resulting mixture was stirred at room temperature for 1 h before it was concentrated in vacuo directly. The residue was dispersed with MTBE (50 mL) and stirred for 30 min before it was filtered. The filter cake was collected and dried in vacuo The intermediate S72 was obtained as a yellow solid (2.19 g, 99%) and used without further purification. HRMS (ESI): calcd for C₃₉H₄₅BrFN₆O₇⁺ M⁺ 807.2512, found 807.2514.

### Step 5:

To a stirring solution of S21 (3.11 g, 2.418 mmol) in DMF (50 mL) at room temperature was added HATU (920 mg, 2.418 mmol). The resulting mixture was stirred at room temperature for 15 min before addition of S₇₂ (2.19 g, 2.418 mmol) and DIPEA (938 mg, 7.254 mmol) in sequence. The resulting mixture was stirred at room temperature for further 1 h before it was concentrated in vacuo directly. The intermediate S73 was obtained as a dark yellow oil (5.02 g, 100%) and used without further purification. HRMS (ESI): calcd for C₁₀₂H₁₄₁FN₁₅O₂₅⁺ M⁺ 1995.0202, found 1995.0208.

### Step 6:

To a stirring solution of S73 (5.02 g, 2.418 mmol) in CH₂Cl₂ (50 mL) at room temperature was added TFA (25 mL). The resulting mixture was stirred at room temperature for 1 h before it was concentrated in vacuo directly. The product A007 was obtained as a yellow solid (2.52 g, 55% over 2 steps) after RP-HPLC purification and lyophilization. HRMS (ESI): calcd for C₈₉H₁₁₉FN₁₅O₂₅⁺ [M+H]⁺ 1816.8480, found 1816.8487.

### Example 357: Synthesis of A018

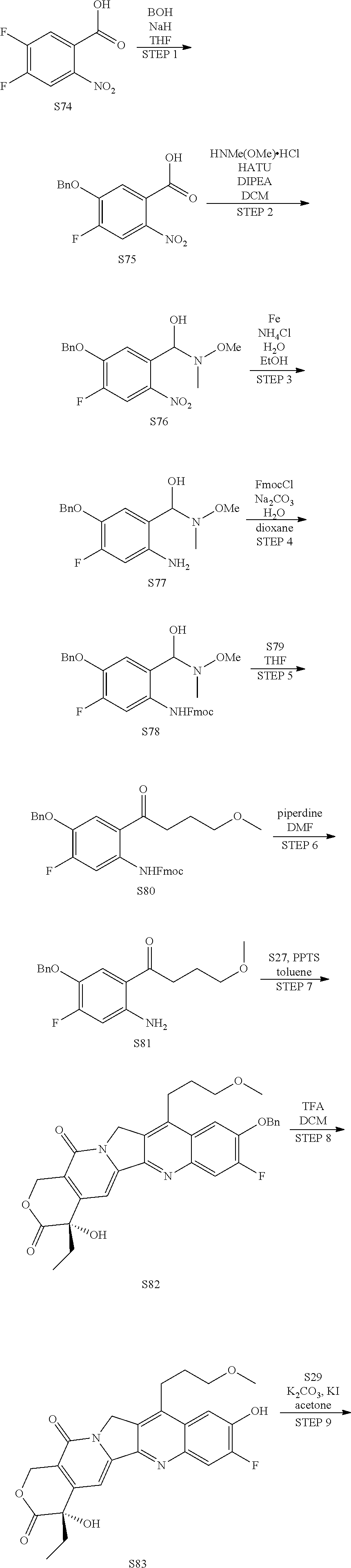

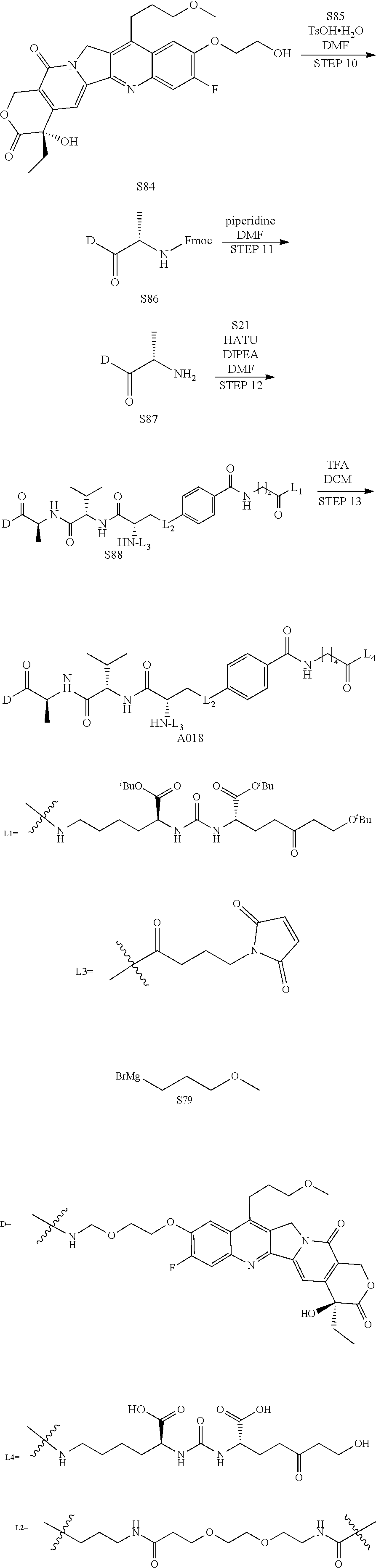

### Step 1:

To a stirring solution of S74 (1.50 g, 7.386 mmol) in THF (50 mL) at 0° C. was added NaH (650 mg, 16.248 mmol, 60% dispersion in mineral oil). The resulting mixture was stirred at 0° C. for 0.5 h before addition of BnOH (2.00 g, 18.4638 mmol). The resulting mixture was stirred at 0° C. for 1 h before it was quenched with NH₄Cl (sat. aq., 30 mL). The layers were separated, and the aqueous layer was extracted with EtOAc (3×30 mL). The combined organic layers were dried (Na₂SO₄) and concentrated in vacuo. The intermediate S75 was obtained as a yellow solid (1.45 g, 67%) after flash column chromatography purification (silica gel, CH₂Cl₂:MeOH=20:1). HRMS (ESI): calcd for C₁₄H₁₁FNO₅⁺ [M+H]⁺ 292.0616, found 292.0620.

To a stirring solution of S75 (1.45 g, 4.977 mmol) and HNMe (OMe)·HCl (583 mg, 5.972 mmol) in CH₂Cl₂ (20 mL) at room temperature were added HATU (2.27 g, 5.972 mmol) and DIPEA (1.93 g, 14.931 mmol). The resulting mixture was stirred at room temperature for 1 h before it was quenched with Na₂CO₃ (5% wt/wt aq., 10 mL). The layers were separated, and the organic layer was washed with HCl (1 M aq., 10 mL) and water (10 mL). The organic layers were dried (Na₂SO₄) and concentrated in vacuo. The intermediate S76 was obtained as a yellow solid (1.60 g, 96%) and used without further purification. HRMS (ESI): calcd for C₁₆H₁₆FN₂O₅⁺ [M+H]⁺ 335.1038, found 335.1039.

### Step 3:

To a stirring solution of S76 (1.60 g, 4.786 mmol) in EtOH/H₂O (30 mL, 50% V/V) at room temperature were added NH₄Cl (2.56 g, 47.861 mmol) and Fe powder (1.87 g, 33.503 mmol). The resulting mixture was heated to 80° C. and stirred for 2 h before it was cooled to room temperature and filtered through a pad of celite. The filtrate was collected and concentrated in vacuo. The intermediate S₇₇ was obtained as a yellow solid (1.45 g, 100%) and used without further purification. HRMS (ESI): calcd for C₁₆H₁₈FN₂O₃⁺ [M+H]⁺ 305.1296, found 305.1300.

### Step 4:

To a stirring solution of S77 (1.45 g, 4.765 mmol) in 1,4-dioxane/H₂O (50 mL, 70% V/V) at 0° C. were added Na₂CO₃ (1.51 g, 14.294 mmol) and FmocCl (3.08 g, 11.9118 mmol). The resulting mixture was warmed to room temperature and stirred for 12 h before it was concentrated in vacuo. The residual aqueous layer was extracted with EtOAc (20 mL×3). The combined organic layers were dried (Na₂SO₄) and concentrated in vacuo. The intermediate S78 was obtained as a white solid (2.50 g, 100%) after flash column chromatography purification (silica gel, hexane:EtOAc=2:1). HRMS (ESI): calcd for C₃₁H₂₈FN₂O₅⁺ [M+H]⁺ 527.1977, found 527.1982.

### Step 5:

To a stirring solution of S78 (2.50 g, 65.604 mmol) in THF (30 mL) at 0° C. was added S79 (9.50 mL, 1.0 M in THF, 9.496 mmol). The resulting mixture was warmed to room temperature and stirred for 6 h before it was quenched with NH₄Cl (sat. aq., 20 mL). The layers were separated, and the aqueous layer was extracted with EtOAc (20 mL×3). The combined organic layers were dried (Na₂SO₄) and concentrated in vacuo. The intermediate S80 was obtained as a yellow oil (2.00 g, 78%) after flash column chromatography purification (silica gel, hexane:EtOAc=2:1). HRMS (ESI): calcd for C₃₃H₃₁FNO₅⁺ [M+H]⁺ 540.2181, found 540.2182.

### Step 6:

To a stirring solution of S80 (2.00 g, 3.706 mmol) in DMF (20 mL) at room temperature was added piperidine (2 mL). The resulting mixture was stirred at room temperature for 30 min before it was concentrated in vacuo directly. The intermediate S81 was obtained as a yellow solid (1.10 g, 94%) after flash column chromatography purification (silica gel, hexane:EtOAc=10:1). HRMS (ESI): calcd for C₁₈H₂₁FNO₃⁺ [M+H]⁺ 318.1500, found 318.1500.

### Step 7:

To a stirring solution of S81 (1.10 g, 3.466 mmol) and 27 (913 mg, 3.466 mmol) in toluene (30 mL) at room temperature was added PPTS (87 mg, 0.347 mmol). The resulting mixture was heated to reflux and stirred overnight before it was cooled to room temperature and filtered. The filter cake was collected and stirred in toluene at reflux for 2 h before it was cooled and filtered. The filter cake was collected and dried in vacuo. The intermediate S82 was obtained as a yellow solid (1.45 g, 77%) after RP-HPLC purification and lyophilization. HRMS (ESI): calcd for C₃₁H₃₀FN₂O₆⁺ [M+H]⁺ 545.2082, found 545.2088.

### Step 8:

To a stirring solution of S82 (3.00 g, 5.509 mmol) in CH₂Cl₂ (40 mL) at room temperature was added TFA (20 mL). The resulting mixture was stirred at room temperature for 1 h before it was concentrated in vacuo directly. The intermediate S83 was obtained as a yellow solid (2.20 g, 87%) after flash column chromatography purification (silica gel, CH₂Cl₂:hexane=20:1). HRMS (ESI): calcd for C₂₄H₂₄FN₂O₆⁺ [M+H]⁺ 455.1613, found 455.1614.

### Step 9:

To a stirring solution of S83 (2.20 g, 4.841 mmol) and S₂₉ (3.63 g, 29.046 mmol) in acetone (50 mL) at room temperature were added K₂CO₃ (2.01 g, 14.523 mmol) and KI (804 mg, 4.841 mmol) in sequence. The resulting mixture was heated to 60° C. and stirred for 20 h before it was cooled to room temperature and concentrated in vacuo directly. The intermediate S84 was obtained as a dark yellow solid (1.33 g, 55%) after flash column chromatography purification (silica gel, DCM:MeOH=10:1). HRMS (ESI): calcd for C₂₆H₂₈FN₂O₇⁺ [M+H]⁺ 499.1875, found 499.1875.

### Step 10:

To a stirring solution of S84 (1.00 g, 2.006 mmol) and 85 (1.53 g, 4.012 mmol) in DMF (30 mL) at room temperature was added p-TsOH·H₂O (191 mg, 1.003 mmol). The resulting mixture was heated to 60° C. and stirred for 2 h before it was cooled to room temperature and concentrated in vacuo directly. The intermediate S86 was obtained as a white solid (1.22 g, 74%) after RP-HPLC purification and lyophilization. HRMS (ESI): calcd for C₄₅H₄₆FN₄O₁₀⁺ [M+H]⁺ 821.3192, found 821.3195.

### Step 11:

To a stirring solution of S86 (1.22 g, 1.486 mmol) in DMF (10 mL) at room temperature was added piperidine (1 mL). The resulting mixture was stirred at room temperature for 30 min before it was concentrated in vacuo directly. The intermediate S87 was obtained as a yellow solid (823 mg, 92%) after RP-HPLC purification and lyophilization. HRMS (ESI): calcd for C₃₀H₃₆FN₄O₈⁺ [M+H]⁺ 599.2512, found 599.2513.

### Step 12:

To a stirring solution of S21 (1.77 g, 1.375 mmol) in DMF (30 mL) at room temperature was added HATU (523 mg, 1.375 mmol). The resulting mixture was stirred at room temperature for 15 min before addition of S₈₆ (823 mg, 1.375 mmol) and DIPEA (533 mg, 4.124 mmol) in sequence. The resulting mixture was stirred at room temperature for further 1 h before it was concentrated in vacuo directly. The intermediate S87 was obtained as a dark yellow oil (2.57 g, 100%) and used without further purification. HRMS (ESI): calcd for C₉₃H₁₃₃FN₁₃O₂₆⁺ [M+H]⁺ 1866.9463, found 1866.9464.

### Step 13:

To a stirring solution of S87 (2.57 g, 1.375 mmol) in CH₂Cl₂ (30 mL) at room temperature was added TFA (15 mL). The resulting mixture was stirred at room temperature for 1 h before it was concentrated in vacuo directly. The product A018 was obtained as a yellow solid (880 mg, 38% over 2 steps) after RP-HPLC purification and lyophilization. HRMS (ESI): calcd for C₈₁H₁₀₉FN₁₃O₂₆⁺ [M+H]⁺ 1698.7585, found 1698.7592.

### Example 358: Synthesis of A019

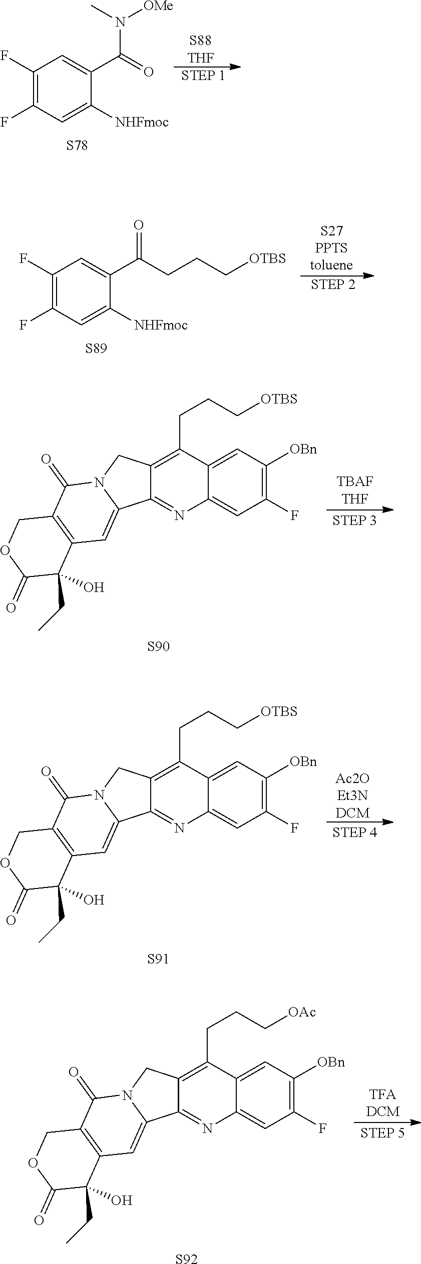

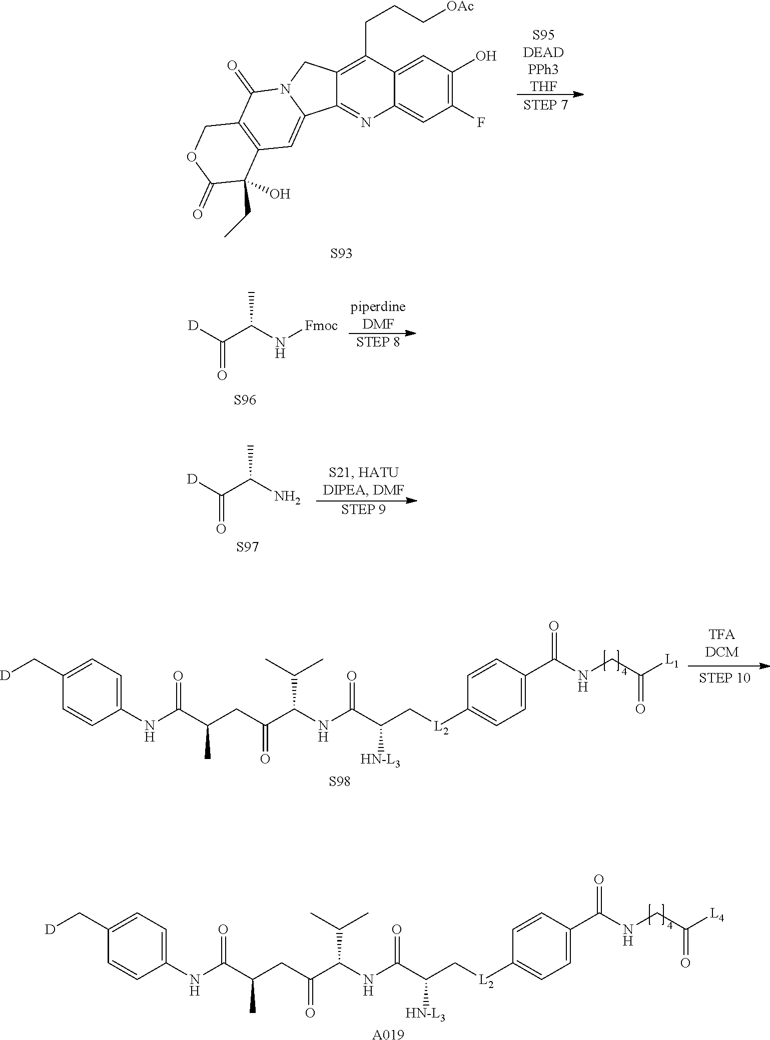

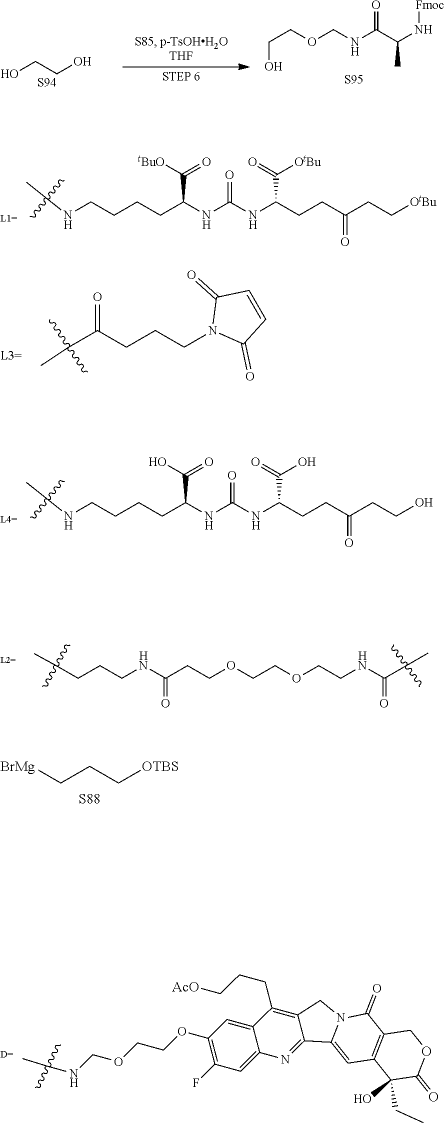

### Step 1:

To a stirring solution of 78 (2.00 g, 3.800 mmol) in THF (30 mL) at 0° C. was added 88 (5.70 mL, 1.0 M in THF, 5.697 mmol). The resulting mixture was warmed to room temperature and stirred for 6 h before it was quenched with NH₄Cl (sat. aq., 20 mL). The layers were separated, and the aqueous layer was extracted with EtOAc (20 mL×3). The combined organic layers were dried (Na₂SO₄) and concentrated in vacuo. The intermediate 89 was obtained as a yellow oil (1.46 g, 60%) after flash column chromatography purification (silica gel, hexane:EtOAc=2:1). HRMS (ESI): calcd for C₃₈H₄₃FNO₅Si⁺ [M+H]⁺ 640.2889, found 640.2896.

### Step 2:

To a stirring solution of 89 (1.46 g, 2.282 mmol) and 27 (601 mg, 2.282 mmol) in toluene (30 mL) at room temperature was added PPTS (58 mg, 0.228 mmol). The resulting mixture was heated to reflux and stirred overnight before it was cooled to room temperature and filtered. The filter cake was collected and stirred in toluene at reflux for 2 h before it was cooled and filtered. The filter cake was collected and dried in vacuo. The intermediate 90 was obtained as a yellow solid (1.09 g, 74%) after RP-HPLC purification and lyophilization. HRMS (ESI): calcd for C₃₆H₄₂FN₂O₆Si⁺ [M+H]⁺ 645.2791, found 645.2793.

### Step 3:

To a stirring solution of 90 (1.09 g, 1.690 mmol) in THF (30 mL) at room temperature was added TBAF (2.54 mL, 1.0 M in THF, 2.536 mmol). The resulting mixture was stirred at temperature and for 30 min before it was quenched with NH₄Cl (sat. aq., 20 mL). The layers were separated, and the aqueous layer was extracted with EtOAc (20 mL×3). The combined organic layers were dried (Na₂SO₄) and concentrated in vacuo. The intermediate 91 was obtained as a yellow solid (724 mg, 80%) after flash column chromatography purification (silica gel, hexane:EtOAc=1:1). HRMS (ESI): calcd for C₃₀H₂₈FN₂O₆⁺ [M+H]⁺ 531.1926, found 531.1934.

### Step 4:

To a stirring solution of 91 (724 mg, 1.365 mmol) in CH₂Cl₂ (10 mL) at room temperature were added Et₃N (553 mg, 5.460 mmol) and Ac₂O (279 mg, 2.730 mmol). The resulting mixture was stirred at room temperature for 2 h before it was quenched with NaHCO₃ (sat. aq., 5 mL). The layers were separated, and the aqueous layer was extracted with CH₂Cl₂ (10 mL×3). The combined organic layers were dried (Na₂SO₄) and concentrated in vacuo. The intermediate 92 was obtained as a yellow oil (655 mg, 84%) after flash column chromatography purification (silica gel, hexane:EtOAc=1:1). HRMS (ESI): calcd for C₃₂H₃₀FN₂O₇⁺ [M+H]⁺ 573.2032, found 573.2035.

### Step 5:

To a stirring solution of 92 (665 mg, 1.144 mmol) in CH₂Cl₂ (10 mL) at room temperature was added TFA (5 mL). The resulting mixture was stirred at room temperature for 1 h before it was concentrated in vacuo directly. The intermediate 93 was obtained as a yellow solid (550 mg, 100%) after flash column chromatography purification (silica gel, CH₂Cl₂:hexane=20:1). HRMS (ESI): calcd for C₂₅H₂₄FN₂O₇⁺ [M+H]⁺ 483.1562, found 483.1563.

### Step 6:

To a stirring solution of 84 (1.00 g, 5.230 mmol) and 94 (650 mg, 10.460 mmol) in THF (30 mL) at room temperature was added p-TsOH·H₂O (498 mg, 2.615 mmol). The resulting mixture was heated to 60° C. and stirred for 2 h before it was cooled to room temperature and concentrated in vacuo directly. The intermediate 95 was obtained as a colorless oil (1.20 g, 60%) after flash column chromatography purification (silica gel, hexane:EtOAc=5:1). HRMS (ESI): calcd for C₂₁H₂₅N2O₅⁺ [M+H]⁺ 385.1758, found 385.1765.

### Step 7:

To a stirring solution of 95 (439 mg, 1.144 mmol) in THF (10 mL) at 0° C. was added PPh₃ (359 mg, 1.373 mmol) and DEAD (240 mg, 1.373 mmol). The resulting mixture was stirred at 0° C. for 15 min before addition of 93 (550 mg, 1.144 mmol). The resulting mixture was stirred at 0° C. for further 1 h before it was with NaCl (sat. aq., 5 mL). The layers were separated, and the aqueous layer was extracted with EtOAc (10 mL×3). The combined organic layers were dried (Na₂SO₄) and concentrated in vacuo. The intermediate 96 was obtained as a yellow oil (760 mg, 78%) after RP-HPLC purification and lyophilization. HRMS (ESI): calcd for C₄₆H₄₆FN₄O₁₁⁺ [M+H]⁺ 849.3142, found 849.3144.

### Step 8:

To a stirring solution of 96 (760 mg, 0.895 mmol) in DMF (10 mL) at room temperature was added piperidine (1 mL). The resulting mixture was stirred at room temperature for 30 min before it was concentrated in vacuo directly. The intermediate 97 was obtained as a yellow solid (400 mg, 71%) after RP-HPLC purification and lyophilization. HRMS (ESI): calcd for C₃₁H₃₆FN₄O₉⁺ [M+H]⁺ 627.2461, found 627.2461.

### Step 9:

To a stirring solution of 21 (820 mg, 0.638 mmol) in DMF (10 mL) at room temperature was added HATU (243 mg, 0.638 mmol). The resulting mixture was stirred at room temperature for 15 min before addition of 97 (400 mg, 0.638 mmol) and DIPEA (248 mg, 1.914 mmol) in sequence. The resulting mixture was stirred at room temperature for further 1 h before it was concentrated in vacuo directly. The intermediate 98 was obtained as a dark yellow oil (1.20 g, 100%) and used without further purification. HRMS (ESI): calcd for C₉₄H₁₃₃FN₁₃O₂₇⁺ [M+H]⁺ 1894.9412, found 1894.9418.

### Step 10:

To a stirring solution of 87 (1.20 g, 0.638 mmol) in CH₂Cl₂ (20 mL) at room temperature was added TFA (10 mL). The resulting mixture was stirred at room temperature for 1 h before it was concentrated in vacuo directly. The product A019 was obtained as a yellow solid (330 mg, 30% over 2 steps) after RP-HPLC purification and lyophilization. HRMS (ESI): calcd for C₈₂H₁₀₉FN₁₃O₂₇⁺ [M+H]⁺ 1726.7534, found 1726.7534.

### Example 359: Synthesis of A020

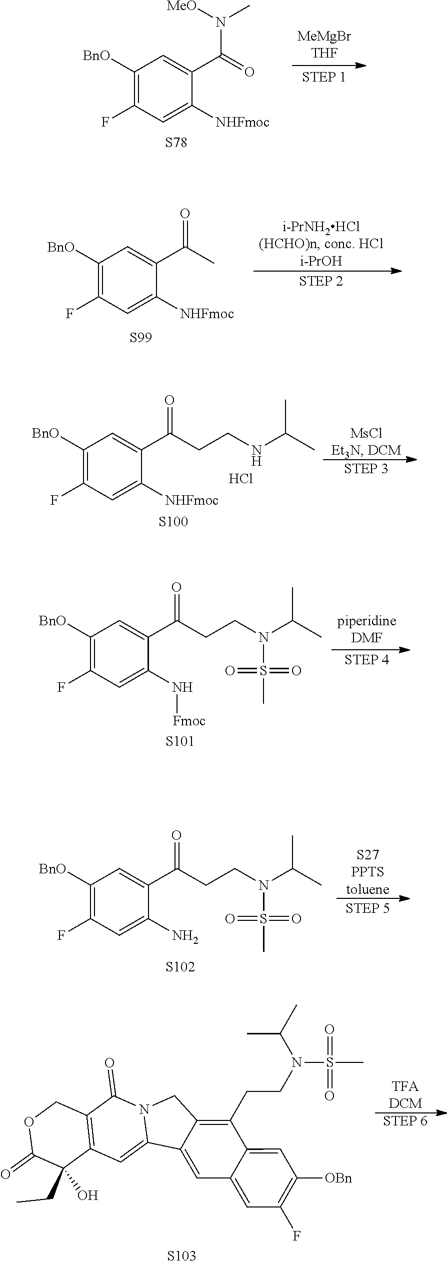

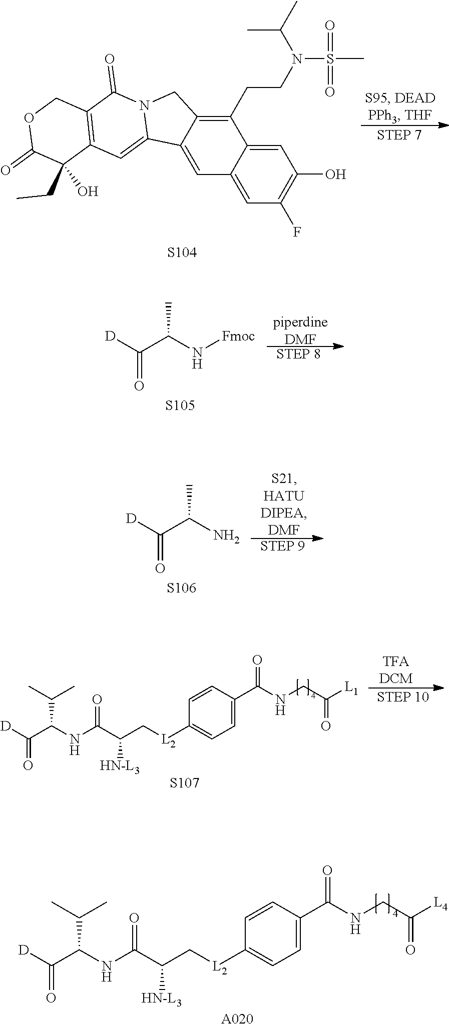

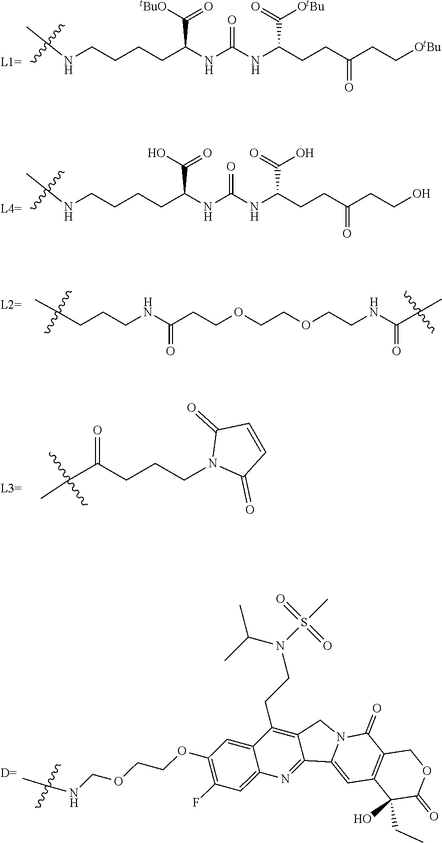 [SMILES: *NCCCC[C@H](NC(=O)N[C@@H](CCC(=O)CCOC(C)(C)C)C(=O)OC(C)(C)C)C(=O)OC(C)(C)C ; *NCCCC[C@H](NC(=O)N[C@@H](CCC(=O)CCO)C(=O)O)C(=O)O ; *CCCNC(=O)CCOCCOCCNC(*)=O ; *C(=O)CCCN1C(=O)C=CC1=O ; *NCOCCOc1cc2c(CCN(C(C)C)S(C)(=O)=O)c3c(nc2cc1F)-c1cc2c(c(=O)n1C3)COC(=O)[C@]2(O)CC]

### Step 1:

To a stirring solution of S78 (5.00 g, 9.496 mmol) in THF (50 mL) at 0° C. was added MeMgBr (4.75 mL, 3.0 mol/L in Et₂O, 14.243 mmol). The resulting mixture was warmed to room temperature and stirred for 1 h before it was quenched with NH₄Cl (sat. aq., 20 mL). The layers were separated, and the aqueous layer was extracted with EtOAc (20 mL×3). The combined organic layers were dried (Na₂SO₄) and concentrated in vacuo. The intermediate S99 was obtained as a yellow solid (4.23 g, 93%) after flash column chromatography purification (silica gel, hexane:EtOAc=5:1). HRMS (ESI): calcd for C₃₀H₂₅FNO₄⁺ [M+H]⁺ 482.1762, found 482.1765.

### Step 2:

To a stirring solution of S99 (4.00 g, 8.307 mmol) and i-Pr₂NH. HCl (3.97 g, 41.535 mmol) and (HCHO)ₙ (1.25 g, 41.535 mmol) in i-PrOH (100 mL) at room temperature was added conc. HCl (1.20 mL). The resulting mixture was heated to reflux and stirred for 12 h before it was cooled to room temperature and quenched with MTBE (200 mL). The resulting mixture was filtered and the filter cake was collected, stirred in refluxing MTBE (50 mL) for 2 h before it was cooled and filtered. The filter cake was collected and dried in vacuo. The intermediate S100 was obtained as a yellow solid (2.50 g, 51%). HRMS (ESI): calcd for C₃₄H₃₄FN₂O₄⁺ [M+H]⁺ 553.2497, found 553.2501.

### Step 3:

To a stirring solution of S100 (2.50 g, 4.244 mmol) in CH₂Cl₂ (50 mL) at 0° C. were added Et₃N (1.72 g, 16.975 mmol) and MsCl (973 mg, 8.488 mmol) in sequence. The resulting mixture stirred at 0° C. for 1 h before it was quenched with NaHCO₃ (sat. aq., 10 mL). The layers were separated, and the aqueous layer was extracted with CH₂Cl₂ (10 mL×3). The combined organic layers were dried (Na₂SO₄) and concentrated in vacuo. The intermediate S101 was obtained as a yellow solid (2.65 g, 99%) after flash column chromatography purification (silica gel, hexane:EtOAc=4:1). HRMS (ESI): calcd for C₃₅H₃₆FN₂O₆S⁺ [M+H]⁺ 631.2273, found 631.2276.

### Step 4:

To a stirring solution of S101 (2.65 g, 4.201 mmol) in DMF (30 mL) at room temperature was added piperidine (3 mL). The resulting mixture was stirred at room temperature for 30 min before it was concentrated in vacuo directly. The intermediate S102 was obtained as a yellow solid (1.69 g, 98%) after flash column chromatography purification (silica gel, hexane:EtOAc=2:1). HRMS (ESI): calcd for C₂₀H₂₆FN₂O₄S⁺ [M+H]⁺ 409.1592, found 409.1595.

### Step 5:

To a stirring solution of S102 (1.50 g, 3.672 mmol) and S27 (967 mg, 3.672 mmol) in toluene (30 mL) at room temperature was added PPTS (185 mg, 0.734 mmol). The resulting mixture was heated to reflux and stirred overnight before it was cooled to room temperature and filtered. The filter cake was collected and stirred in refluxing toluene for 2 h before it was cooled and filtered. The filter cake was collected and dried in vacuo. The intermediate S103 was obtained as a yellow solid (1.05 g, 45%) after RP-HPLC purification and lyophilization. HRMS (ESI): calcd for C₃₃H₃₅FN₃O₇S⁺ [M+H]⁺ 636.2174, found 636.2172.

### Step 6:

To a stirring solution of S103 (1.00 g, 1.573 mmol) in CH₂Cl₂ (20 mL) at room temperature was added TFA (10 mL). The resulting mixture was stirred at room temperature for 1 h before it was concentrated in vacuo directly. The intermediate S104 was obtained as a yellow solid (858 mg, 100%) after flash column chromatography purification (silica gel, CH₂Cl₂:hexane=20:1). HRMS (ESI): calcd for C₂₆H₂₉FN₃O₇S⁺ [M+H]⁺ 546.1705, found 546.1706.

### Step 7:

To a stirring solution of S95 (353 mg, 0.917 mmol) in THF (10 mL) at 0° C. was added PPh₃ (289 mg, 1.100 mmol) and DEAD (192 mg, 1.100 mmol). The resulting mixture was stirred at 0° C. for 15 min before addition of S104 (500 mg, 0.917 mmol). The resulting mixture was stirred at 0° C. for further 1 h before it was with NaCl (sat. aq., 5 mL). The layers were separated, and the aqueous layer was extracted with EtOAc (10 mL×3). The combined organic layers were dried (Na₂SO₄) and concentrated in vacuo. The intermediate S105 was obtained as a yellow solid (433 mg, 52%) after RP-HPLC purification and lyophilization. HRMS (ESI): calcd for C₄₇H₅₁FN₅O₁₁S⁺ [M+H]⁺ 912.3284, found 912.3285.

### Step 8:

To a stirring solution of S105 (433 mg, 0.475 mmol) in DMF (10 mL) at room temperature was added piperidine (1 mL). The resulting mixture was stirred at room temperature for 30 min before it was concentrated in vacuo directly. The intermediate S106 was obtained as a yellow solid (300 mg, 92%) after RP-HPLC purification and lyophilization. HRMS (ESI): calcd for C₃₂H₄₁FN₅O₉S⁺ [M+H]⁺ 690.2604, found 690.2609.

### Step 9:

To a stirring solution of S21 (466 mg, 0.362 mmol) in DMF (10 mL) at room temperature was added HATU (138 mg, 0.362 mmol). The resulting mixture was stirred at room temperature for 15 min before addition of S₁₀₇ (250 mg, 0.362 mmol) and DIPEA (141 mg, 1.087 mmol) in sequence. The resulting mixture was stirred at room temperature for further 1 h before it was concentrated in vacuo directly. The intermediate S108 was obtained as a dark yellow oil (709 mg, 100%) and used without further purification. HRMS (ESI): calcd for C₉₅H₁₃₈FN₁₄O₂₇S⁺ [M+H]⁺ 1957.9555, found 1957.9561. STEP 10:

To a stirring solution of S108 (709 mg, 0.362 mmol) in CH₂Cl₂ (3 mL) at room temperature was added TFA (1.5 mL). The resulting mixture was stirred at room temperature for 1 h before it was concentrated in vacuo directly. The product A020 was obtained as a yellow solid (250 mg, 39% over 2 steps) after RP-HPLC purification and lyophilization. HRMS (ESI): calcd for C₈₃H₁₁₄FN₁₄O₂₇S⁺ [M⁺ H]⁺ 1789.7677, found 1789.7686.

### Example 360: Synthesis of A021

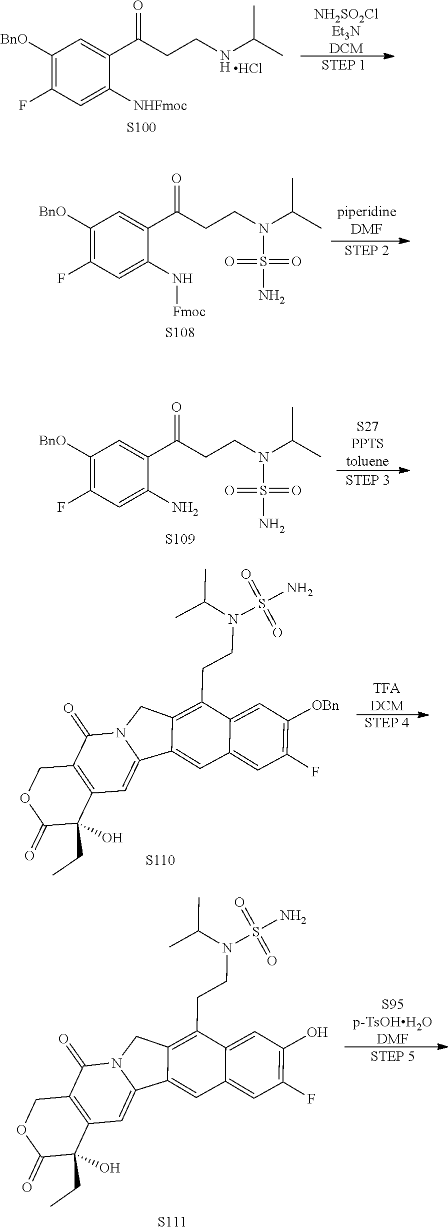

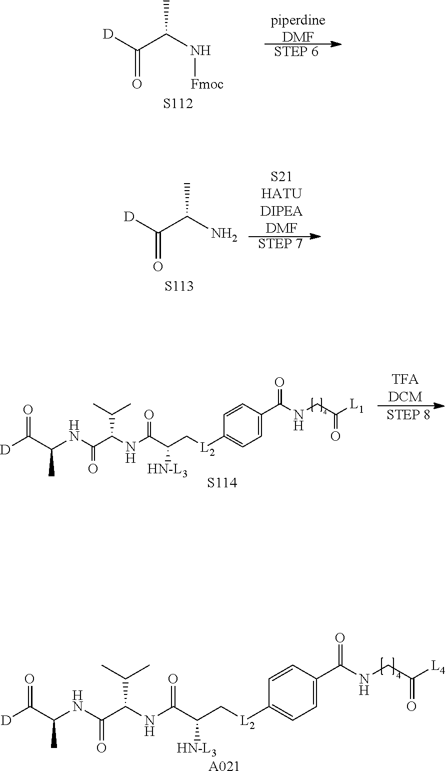

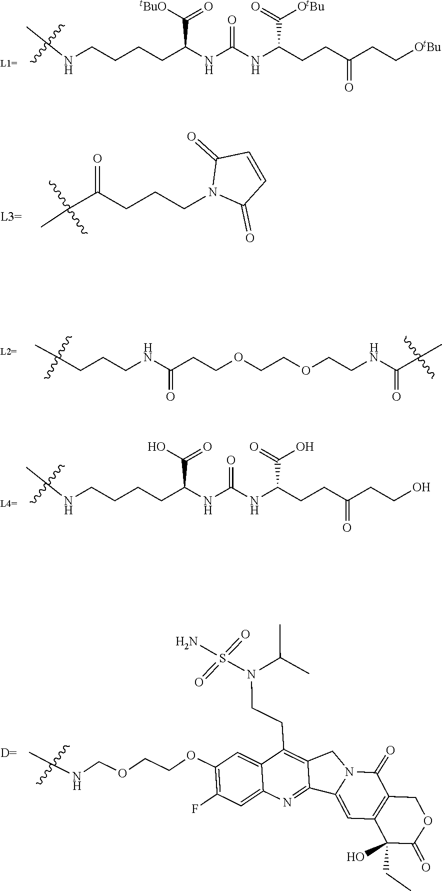 [SMILES: *NCCCC[C@H](NC(=O)N[C@@H](CCC(=O)CCOC(C)(C)C)C(=O)OC(C)(C)C)C(=O)OC(C)(C)C ; *C(=O)CCCN1C(=O)C=CC1=O ; *CCCNC(=O)CCOCCOCCNC(*)=O ; *NCCCC[C@H](NC(=O)N[C@@H](CCC(=O)CCO)C(=O)O)C(=O)O ; *NCOCCOc1cc2c(CCN(C(C)C)S(N)(=O)=O)c3c(nc2cc1F)-c1cc2c(c(=O)n1C3)COC(=O)[C@]2(O)CC]

Step 1˜STEP 8: Intermediate S108˜S114 and product A021 were prepared according to the procedures in A020. A021: HRMS (ESI): calcd for C₈₂H₁₁₃FN₁₅O₂₇S⁺ [M+H]⁺ 1790.7630, found 1790.7638.

### Example 361: Synthesis of A023

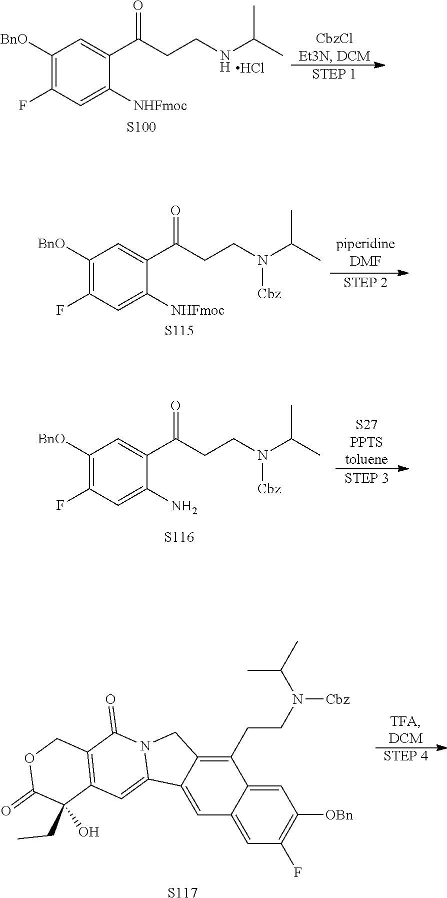

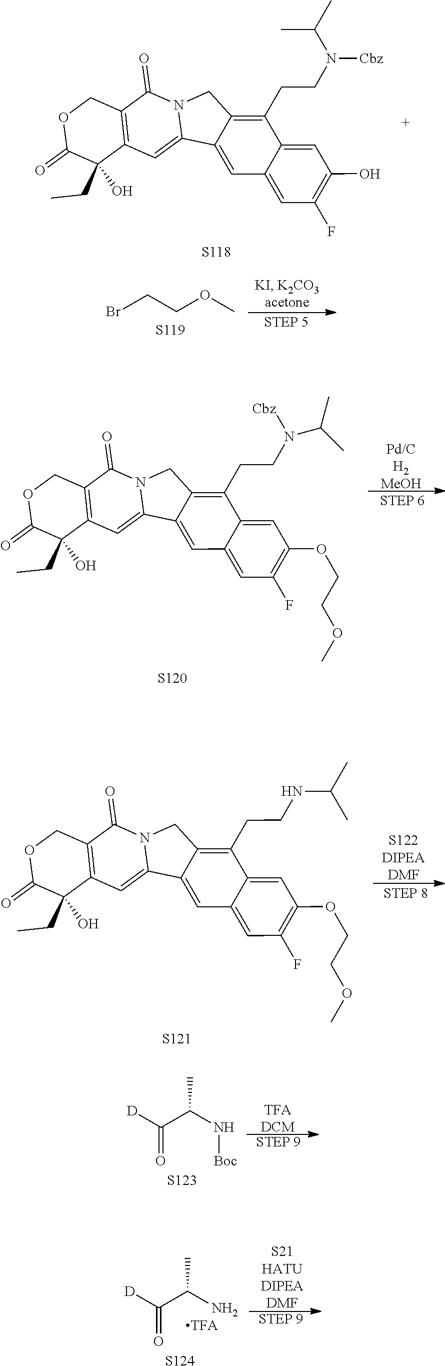

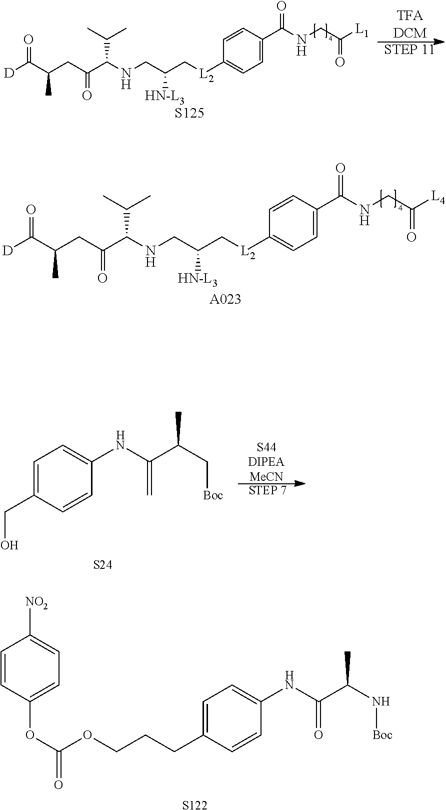

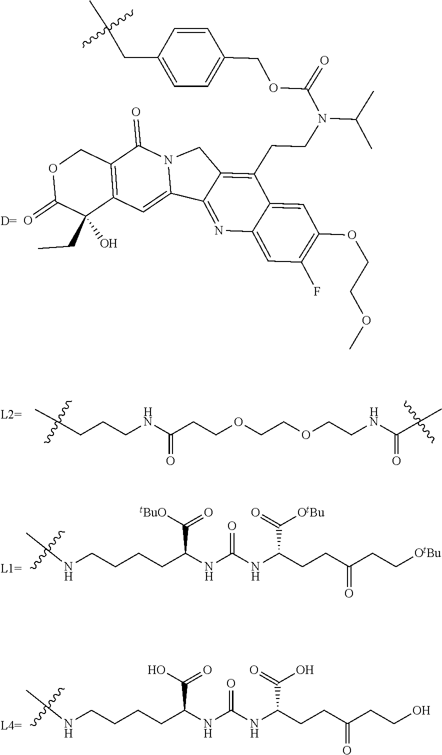 [SMILES: *Cc1ccc(COC(=O)N(CCc2c3c(nc4cc(F)c(OCCOC)cc24)-c2cc4c(c(=O)n2C3)COC(=O)[C@]4(O)CC)C(C)C)cc1 ; *CCCNC(=O)CCOCCOCCNC(*)=O ; *NCCCC[C@H](NC(=O)N[C@@H](CCC(=O)CCOC(C)(C)C)C(=O)OC(C)(C)C)C(=O)OC(C)(C)C ; *NCCCC[C@H](NC(=O)N[C@@H](CCC(=O)CCO)C(=O)O)C(=O)O]

### Step 1:

To a stirring solution of S100 (5.00 g, 8.488 mmol) in CH₂Cl₂ (100 mL) at 0° C. were added Et₃N (1.72 g, 16.975 mmol) and CbzCl (2.17 g, 12.731 mmol) in sequence. The resulting mixture was stirred at 0° C. for 2 h before it was quenched with NaHCO₃ (sat. aq., 20 mL). The layers were separated, and the aqueous layer was extracted with CH₂Cl₂ (20 mL×3). The combined organic layers were dried (Na₂SO₄) and concentrated in vacuo. The intermediate S115 was obtained as a yellow solid (5.80 g, 99%) after flash column chromatography purification (silica gel, hexane:EtOAc=5:1). HRMS (ESI): calcd for C₄₂H₄₀FN₂O₆⁺ [M+H]⁺ 687.2865, found 687.2870.

To a stirring solution of S115 (5.80 g, 8.445 mmol) in DMF (30 mL) at room temperature was added piperidine (3 mL). The resulting mixture was stirred at room temperature for 30 min before it was concentrated in vacuo directly. The intermediate S116 was obtained as a yellow solid (3.90 g, 99%) after flash column chromatography purification (silica gel, hexane:EtOAc=2:1). HRMS (ESI): calcd for C₂₇H₃₀FN₂O₄⁺ [M+H]⁺ 465.2184, found 465.2185.

### Step 3:

To a stirring solution of S116 (3.90 g, 8.395 mmol) and S₂₇ (2.21 g, 8.395 mmol) in toluene (100 mL) at room temperature was added PPTS (422 mg, 1.679 mmol). The resulting mixture was heated to reflux and stirred overnight before it was cooled to room temperature and filtered. The filter cake was collected and stirred in refluxing toluene for 2 h before it was cooled and filtered. The filter cake was collected and dried in vacuo. The intermediate S117 was obtained as a yellow solid (4.17 g, 72%) after RP-HPLC purification and lyophilization. HRMS (ESI): calcd for C₄₀H₃₉FN₃O₇⁺ [M+H]⁺ 692.2767, found 692.2767.

### Step 4:

To a stirring solution of S117 (4.17 g, 6.08 mmol) in CH₂Cl₂ (50 mL) at room temperature was added TFA (25 mL). The resulting mixture was stirred at room temperature for 1 h before it was concentrated in vacuo directly. The intermediate S118 was obtained as a yellow solid (3.50 g, 97%) after flash column chromatography purification (silica gel, CH₂Cl₂:hexane=20:1). HRMS (ESI): calcd for C₃₃H₃₃FN₃O₇⁺ [M+H]⁺ 602.2297, found 602.2298.

### Step 5:

To a stirring solution of S118 (3.50 g, 5.818 mmol) and S₁₁₉ (4.85 g, 34.908 mmol) in acetone (50 mL) at room temperature were added K₂CO₃ (2.41 g, 17.44 mmol) and KI (966 mg, 5.818 mmol) in sequence. The resulting mixture was heated to 60° C. and stirred for 20 h before it was cooled to room temperature and concentrated in vacuo directly. The intermediate S120 was obtained as a dark yellow solid (3.20 g, 83%) after flash column chromatography purification (silica gel, DCM:MeOH=10:1). HRMS (ESI): calcd for C₃₆H₃₉FN₃O₈⁺ [M+H]⁺ 660.2716, found 660.2723.

### Step 6:

To a stirring solution of S121 (3.00 g, 4.547 mmol) in MeOH (30 mL) at room temperature was added Pd/C (3.00 g, 10% wt/wt). The resulting mixture was stirred at room temperature under H₂ atmosphere for 1 h before it was filtered through a pad of celite. The filtrate was collected and concentrated in vacuo. The intermediate S121 was obtained as a yellow oil (2.39 g, 100%) and used without further purification. HRMS (ESI): calcd for C₂₈H₃₃FN₃O₆⁺ [M+H]⁺ 526.2348, found 526.2348.

### Step 7:

To a stirring solution of S24 (5.00 g, 16.987 mmol) in MeCN (100 mL) at 0° C. were added DIPEA (3.29 g, 25.480 mmol) and S₄₄ (4.11 g, 20.384 mmol) in sequence. The resulting mixture was stirred at 0° C. for 3 h before it was quenched with NaHCO₃ (sat. aq., 20 mL). The layers were separated, and the aqueous layer was extracted with EtOAc (20 mL×3). The combined organic layers were dried (Na₂SO₄) and concentrated in vacuo. The intermediate S122 was obtained as a yellow solid (6.33 g, 81%) after flash column chromatography purification (silica gel, hexane:EtOAc=1:1). HRMS (ESI): calcd for C₂₂H₂₆N3O₈⁺ [M+H]⁺ 460.1714, found 460.1720.

### Step 8:

To a stirring solution of S121 (2.39 g, 4.547 mmol) in DMF (50 mL) at room temperature were added DIPEA (882 mg, 6.821 mmol) and S₁₂₂ (2.51 g, 5.547 mmol) in sequence. The resulting mixture was stirred at room temperature for 2 h before it was cooled to room temperature and concentrated in vacuo directly. The intermediate S123 was obtained as a dark yellow solid (3.45 g, 89%) after flash column chromatography purification (silica gel, DCM:MeOH=20:1). HRMS (ESI): calcd for C₄₄H₅₃FN₅O₁₁⁺ [M+H]⁺ 846.3720, found 846.3721.

### Step 9:

To a stirring solution of S123 (3.45 g, 4.078 mmol) in CH₂Cl₂ (40 mL) at room temperature was added TFA (20 mL). The resulting mixture was stirred at room temperature for 1 h before it was concentrated in vacuo directly. The intermediate S124 was obtained as a yellow solid (3.50 g, 100%) after trituration with MTBE (100 mL). HRMS (ESI): calcd for C₃₉H₄₅FN₅O₉⁺ [M+H]⁺ 746.3196, found 746.3196.

### Step 10:

To a stirring solution of S21 (5.24 g, 4.071 mmol) in DMF (100 mL) at room temperature was added HATU (1.55 g, 4.071 mmol). The resulting mixture was stirred at room temperature for 15 min before addition of S124 (3.50 g, 4.071 mmol) and DIPEA (1.58 g, 12.212 mmol) in sequence. The resulting mixture was stirred at room temperature for further 1 h before it was concentrated in vacuo directly. The intermediate S125 was obtained as a dark yellow oil (8.20 g, 100%) and used without further purification. HRMS (ESI): calcd for C₁₀₂H₁₄₂FN₁₄O₂₇⁺ [M+H]⁺ 2014.0147, found 2014.0152.

### Step 11:

To a stirring solution of S125 (8.20 g, 4.071 mmol) in CH₂Cl₂ (100 mL) at room temperature was added TFA (50 mL). The resulting mixture was stirred at room temperature for 1 h before it was concentrated in vacuo directly. The product A023 was obtained as a yellow solid (3.36 g, 45% over 2 steps) after RP-HPLC purification and lyophilization. HRMS (ESI): calcd for C₈₉H₁₁₆FN₁₄O₂₇⁺ [M+H]⁺ 1831.8113, found 1831.8120.

### Example 362: Synthesis of A022

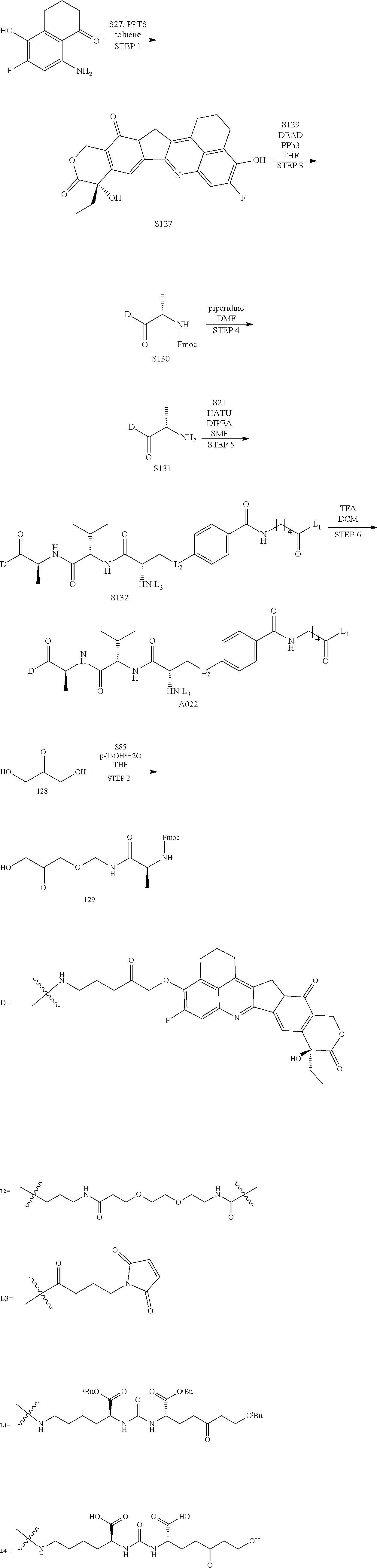

### Step 1:

To a stirring solution of S126 (5.00 g, 25.616 mmol) and S₂₇ (6.74 g, 25.616 mmol) in toluene (100 mL) at room temperature was added PPTS (3.22 g, 12.808 mmol). The resulting mixture was heated to reflux and stirred overnight before it was cooled to room temperature and filtered. The filter cake was collected and stirred in refluxing toluene for 3 h before it was cooled and filtered. The filter cake was collected and dried in vacuo. The intermediate S127 was obtained as a yellow solid (7.11 g, 66%) after RP-HPLC purification and lyophilization. HRMS (ESI): calcd for C₂₃H₂₀FN₂O₅⁺ [M+H]⁺ 423.1351, found 423.1355.

### Step 2:

To a stirring solution of S128 (10.00 g, 111.012 mmol) and S₉₄ (84.91 g, 222.025 mmol) in THF (500 mL) at room temperature was added p-TsOH·H₂O (10.56 g, 55.506 mmol). The resulting mixture was heated to 60° C. and stirred for 2 h before it was cooled to room temperature and concentrated in vacuo directly. The intermediate S95 was obtained as a colorless oil (12.12 g, 24%) after flash column chromatography purification (silica gel, hexane:EtOAc=5:1). HRMS (ESI): calcd for C₂₂H₂₅N2O₆⁺ [M+H]⁺ 413.1707, found 413.1713.

### Step 3:

To a stirring solution of S129 (1.95 g, 4.735 mmol) in THF (50 mL) at 0° C. was added PPh₃ (1.49 g, 5.682 mmol) and DEAD (990 mg, 5.682 mmol). The resulting mixture was stirred at 0° C. for 15 min before addition of 127 (2.00 g, 4.735 mmol). The resulting mixture was stirred at 0° C. for further 1 h before it was with NaCl (sat. aq., 20 mL). The layers were separated, and the aqueous layer was extracted with EtOAc (20 mL×3). The combined organic layers were dried (Na₂SO₄) and concentrated in vacuo. The intermediate S130 was obtained as a yellow oil (1.45 g, 38%) after RP-HPLC purification and lyophilization. HRMS (ESI): calcd for C₄₄H₄₂FN₄O₁₀⁺ [M+H]⁺ 805.2879, found 805.2880.

### Step 4:

To a stirring solution of S130 (1.45 g, 1.802 mmol) in DMF (20 mL) at room temperature was added piperidine (2 mL). The resulting mixture was stirred at room temperature for 30 min before it was concentrated in vacuo directly. The intermediate S131 was obtained as a yellow solid (620 mg, 59%) after RP-HPLC purification and lyophilization. HRMS (ESI): calcd for C₂₉H₃₂FN₄O₈⁺ [M+H]⁺ 583.2199, found 583.2204.

### Step 5:

To a stirring solution of S21 (1.37 g, 1.064 mmol) in DMF (20 mL) at room temperature was added HATU (405 mg, 1.064 mmol). The resulting mixture was stirred at room temperature for 15 min before addition of S₁₃₁ (620 mg, 1.064 mmol) and DIPEA (413 mg, 3.192 mmol) in sequence. The resulting mixture was stirred at room temperature for further 1 h before it was concentrated in vacuo directly. The intermediate S132 was obtained as a dark yellow oil (1.97 g, 100%) and used without further purification. HRMS (ESI): calcd for C₉₂H₁₂₉FN₁₃O₂₆⁺ [M+H]⁺ 1850.9150, found 1850.9151.

### Step 6:

To a stirring solution of S131 (1.97 g, 1.064 mmol) in CH₂Cl₂ (30 mL) at room temperature was added TFA (15 mL). The resulting mixture was stirred at room temperature for 1 h before it was concentrated in vacuo directly. The product A022 was obtained as a yellow solid (448 mg, 25% over 2 steps) after RP-HPLC purification and lyophilization. HRMS (ESI): calcd for C₈₀H₁₀₅FN₁₃O₂₆⁺ [M+H]⁺ 1682.7272, found 1682.7277.

### Example 363: Synthesis of A026

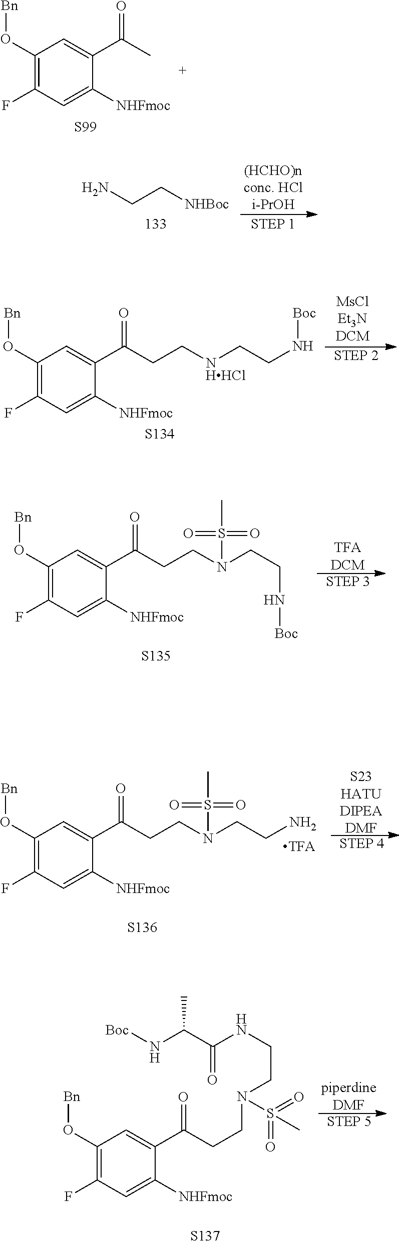

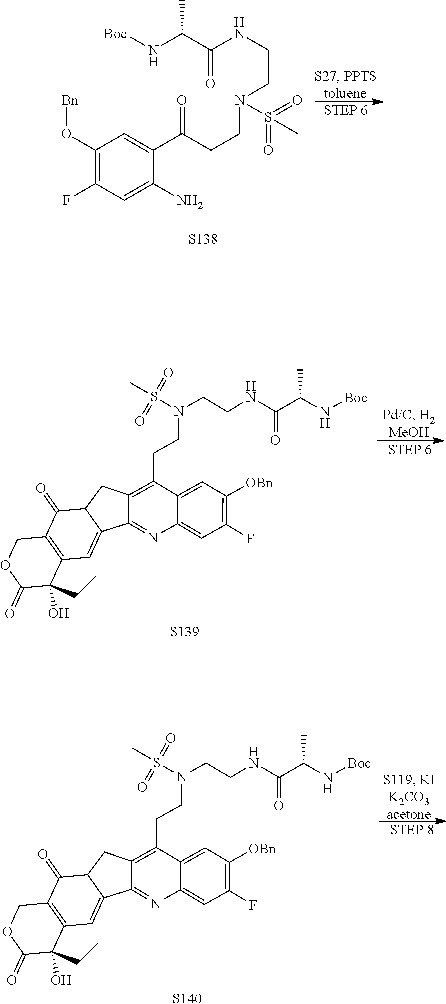 [SMILES: C[C@@H](NC(=O)OC(C)(C)C)C(=O)NCCN(CCC(=O)c1cc(OCc2ccccc2)c(F)cc1N)S(C)(=O)=O ; "S138" ; CC[C@@]1(O)C(=O)OCC2=C1C=C1c3nc4cc(F)c(OCc5ccccc5)cc4c(CCN(CCNC(=O)[C@H](C)NC(=O)OC(C)(C)C)S(C)(=O)=O)c3CC1C2=O ; "S139" ; CC[C@@]1(O)C(=O)OCC2=C1C=C1c3nc4cc(F)c(OCc5ccccc5)cc4c(CCN(CCNC(=O)[C@H](C)NC(=O)OC(C)(C)C)S(C)(=O)=O)c3CC1C2=O ; "S140"]

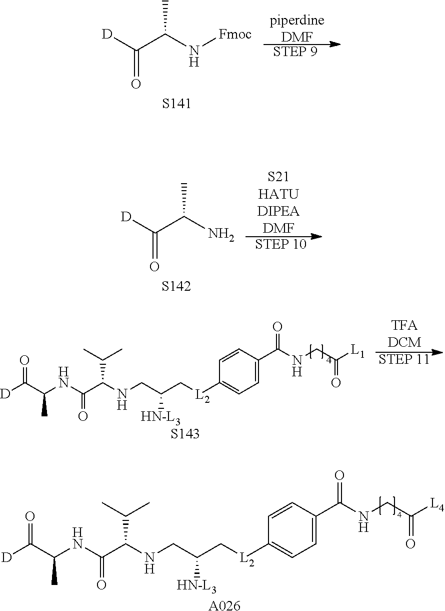

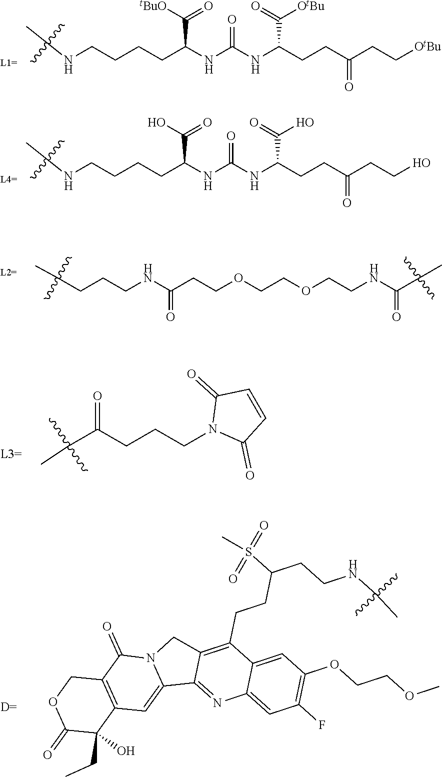 [SMILES: *NCCCC[C@H](NC(=O)N[C@@H](CCC(=O)CCOC(C)(C)C)C(=O)OC(C)(C)C)C(=O)OC(C)(C)C ; *NCCCC[C@H](NC(=O)N[C@@H](CCC(=O)CCO)C(=O)O)C(=O)O ; *CCCNC(=O)CCOCCOCCNC(*)=O ; *C(=O)CCCN1C(=O)C=CC1=O ; *NCCC(CCc1c2c(nc3cc(F)c(OCCOC)cc13)-c1cc3c(c(=O)n1C2)COC(=O)[C@]3(O)CC)S(C)(=O)=O]

### Step 1:

To a stirring solution of S99 (5.00 g, 10.384 mmol) and S133 (8.32 g, 51.919 mmol) and (HCHO) n (1.56 g, 51.919 mmol) in i-PrOH (120 mL) at room temperature was added conc. HCl (1.50 mL). The resulting mixture was heated to reflux and stirred for 12 h before it was cooled to room temperature and quenched with MTBE (200 mL). The resulting mixture was filtered and the filter cake was collected, stirred in refluxing MTBE (50 mL) for 2 h before it was cooled and filtered. The filter cake was collected and dried in vacuo. The intermediate S134 was obtained as a yellow solid (1.56 g, 22%). HRMS (ESI): calcd for C₃₈H₄₁FN₃O₆⁺ [M+H]⁺ 654.2974, found 654.2975.

To a stirring solution of S134 (1.56 g, 2.260 mmol) in CH₂Cl₂ (50 mL) at 0° C. were added Et₃N (915 mg, 9.041 mmol) and MsCl (518 mg, 4.520 mmol) in sequence. The resulting mixture stirred at 0° C. for 1 h before it was quenched with NaHCO₃ (sat. aq., 10 mL). The layers were separated, and the aqueous layer was extracted with CH₂Cl₂ (10 mL×3). The combined organic layers were dried (Na₂SO₄) and concentrated in vacuo. The intermediate S135 was obtained as a yellow solid (1.11 g, 67%) after flash column chromatography purification (silica gel, hexane:EtOAc=2:1). HRMS (ESI): calcd for C₃₉H₄₃FN₃O₈S⁺ [M+H]⁺ 732.2749, found 732.2755.

### Step 3:

To a stirring solution of S135 (1.11 g, 1.517 mmol) in CH₂Cl₂ (40 mL) at room temperature was added TFA (20 mL). The resulting mixture was stirred at room temperature for 1 h before it was concentrated in vacuo directly. The residue was dispersed with MTBE (100 mL) and stirred for 30 min before it was filtered. The filter cake was collected and dried in vacuo. The intermediate S136 was obtained as a yellow solid (1.12 g, 99%) and used without further purification. HRMS (ESI): calcd for C₃₄H₃₅FN₃O₆S⁺ [M+H]⁺ 632.2225, found 632.2228.

### Step 4:

To a stirring solution of S23 (341 mg, 1.802 mmol) in DMF (15 mL) at room temperature were added HATU (686 mg, 1.802 mmol) and DIPEA (583 mg, 4.506 mmol) in sequence. The resulting mixture was stirred at room temperature for 30 min before addition of S₁₃₆ (1.12 g, 1.502 mmol). The resulting mixture was stirred at room temperature for further 1 h before it was quenched with NaHCO₃ (sat. aq., 10 mL). The mixture layers was extracted with CH₂Cl₂ (20 mL×3). The combined organic layers were dried (Na₂SO₄) and concentrated in vacuo. The intermediate S137 was obtained as a yellow oil (1.02 mg, 85%) after flash column chromatography purification (silica gel, hexane:EtOAc=1:1). HRMS (ESI): calcd for C₄₂H₄₈FN₄O₉S⁺ [M+H]⁺ 803.3121, found 803.3122.

### Step 5:

To a stirring solution of S137 (1.02 g, 1.270 mmol) in DMF (10 mL) at room temperature was added piperidine (1 mL). The resulting mixture was stirred at room temperature for 30 min before it was concentrated in vacuo directly. The intermediate S138 was obtained as a yellow solid (688 mg, 93%) after RP-HPLC purification and lyophilization. HRMS (ESI): calcd for C₂₇H₃₈FN₄O₇S⁺ [M+H]⁺ 581.2440, found 581.2440.

### Step 6:

To a stirring solution of S138 (680 mg, 1.171 mmol) and S₂₇ (309 mg, 1.171 mmol) in toluene (10 mL) at room temperature was added PPTS (69 mg, 0.234 mmol). The resulting mixture was heated to reflux and stirred overnight before it was cooled to room temperature and filtered. The filter cake was collected and stirred in refluxing toluene for 3 h before it was cooled and filtered. The filter cake was collected and dried in vacuo. The intermediate S139 was obtained as a white solid (500 mg, 52%) after RP-HPLC purification and lyophilization. HRMS (ESI): calcd for C₄₀H₄₇FN₅O₁₀S⁺ [M+H]⁺ 808.3022, found 808.3026.

### Step 7:

To a stirring solution of S139 (500 mg, 0.619 mmol) in MeOH (10 mL) at room temperature was added Pd/C (50 mg, 10% wt/wt). The resulting mixture was stirred at room temperature under H₂ atmosphere for 5 h before it was filtered through a pad of celite. The filtrate was collected and concentrated in vacuo. The intermediate S140 was obtained as a yellow solid (383 mg, 86%) after flash column chromatography purification (silica gel, CH₂Cl₂:hexane=10:1). HRMS (ESI): calcd for C₃₃H₄₁FN₅O₁₀S⁺ [M+H]⁺ 718.2553, found 718.2554.

### Step 8:

To a stirring solution of S140 (380 mg, 0.529 mmol) and S₁₁₉ (442 mg, 3.177 mmol) in acetone (10 mL) at room temperature were added K₂CO₃ (220 mg, 1.588 mmol) and KI (88 mg, 0.529 mmol) in sequence. The resulting mixture was heated to 60° C. and stirred for 20 h before it was cooled to room temperature and concentrated in vacuo directly. The intermediate S141 was obtained as a yellow solid (190 mg, 46%) after flash column chromatography purification (silica gel, DCM:MeOH=10:1). HRMS (ESI): calcd for C₃₆H₄₇FN₅O₁₁S⁺ [M+H]⁺ 776.2971, found 776.2978.

### Step 9:

To a stirring solution of S141 (190 mg, 0.245 mmol) in CH₂Cl₂ (5 mL) at room temperature was added TFA (2 mL). The resulting mixture was stirred at room temperature for 1 h before it was concentrated in vacuo directly. The intermediate S142 was obtained as a yellow solid (150 mg, 78%) after RP-HPLC purification and lyophilization. HRMS (ESI): calcd for C₃₁H₃₉FN₅O₉S⁺ [M+H]⁺ 676.2447, found 676.2455.

### Step 10:

To a stirring solution of S21 (244 mg, 0.190 mmol) in DMF (10 mL) at room temperature was added HATU (72 mg, 0.190 mmol). The resulting mixture was stirred at room temperature for 15 min before addition of S₁₄₂ (150 mg, 0.190 mmol) and DIPEA (74 mg, 0.570 mmol) in sequence. The resulting mixture was stirred at room temperature for further 1 h before it was concentrated in vacuo directly. The intermediate S143 was obtained as a dark yellow oil (369 mg, 100%) and used without further purification. HRMS (ESI): calcd for C₉₄H₁₃₆FN₁₄O₂₇S⁺ [M+H]⁺ 1943.9399, found 1943.9402.

### Step 11:

To a stirring solution of S143 (369 mg, 0.190 mmol) in CH₂Cl₂ (5 mL) at room temperature was added TFA (2 mL). The resulting mixture was stirred at room temperature for 1 h before it was concentrated in vacuo directly. The product A026 was obtained as a yellow solid (124 mg, 36% over 2 steps) after RP-HPLC purification and lyophilization. HRMS (ESI): calcd for C₈₂H₁₁₂FN₁₄O₂₇S⁺ [M+H]⁺ 1775.7521, found 1775.7530.

### Example 364: Synthesis of A027

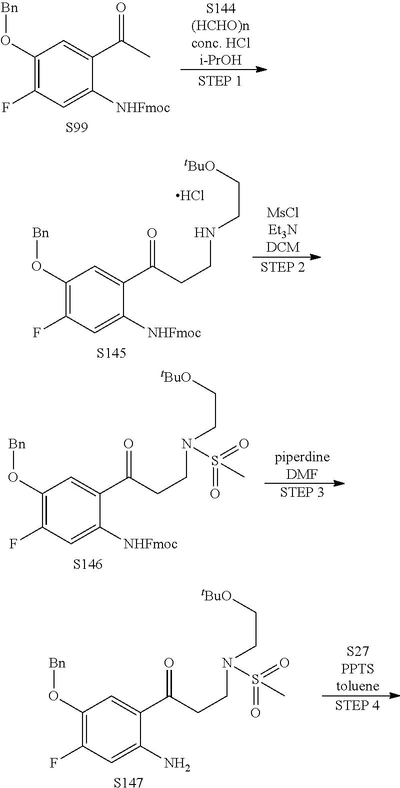

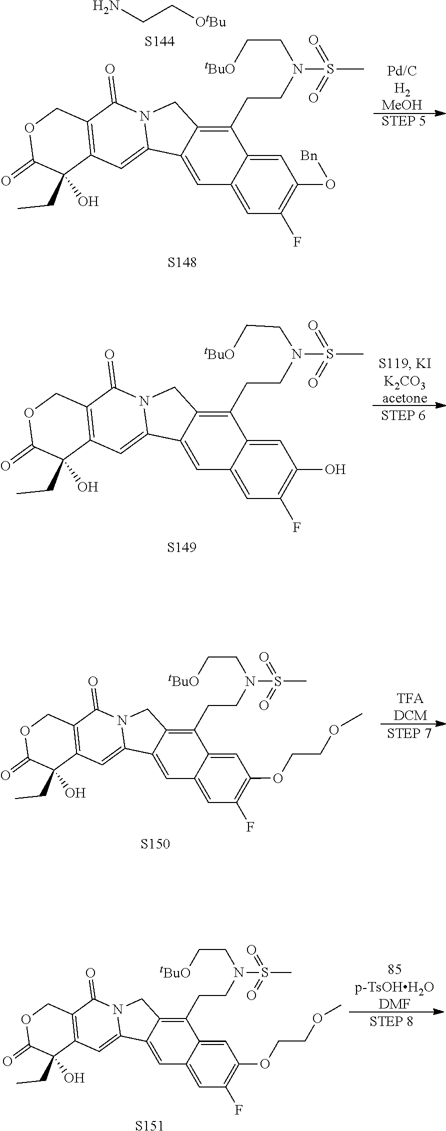

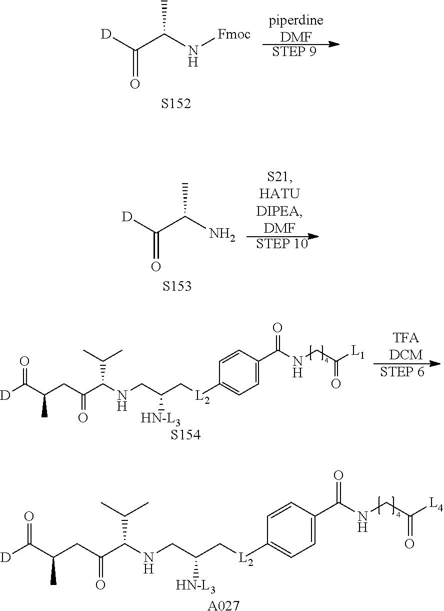

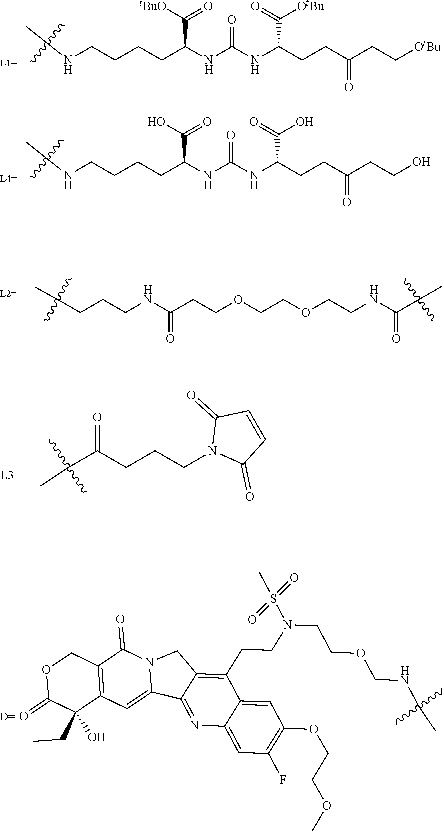 [SMILES: *NCCCC[C@H](NC(=O)N[C@@H](CCC(=O)CCOC(C)(C)C)C(=O)OC(C)(C)C)C(=O)OC(C)(C)C ; *NCCCC[C@H](NC(=O)N[C@@H](CCC(=O)CCO)C(=O)O)C(=O)O ; *CCCNC(=O)CCOCCOCCNC(*)=O ; *C(=O)CCCN1C(=O)C=CC1=O ; *NCOCCN(CCc1c2c(nc3cc(F)c(OCCOC)cc13)-c1cc3c(c(=O)n1C2)COC(=O)[C@]3(O)CC)S(C)(=O)=O]

### Step 1:

To a stirring solution of S99 (10.00 g, 20.768 mmol) and S144 (12.17 g, 103.838 mmol) and (HCHO) n (3.12 g, 103.838 mmol) in i-PrOH (250 mL) at room temperature was added conc. HCl (3 mL). The resulting mixture was heated to reflux and stirred for 12 h before it was cooled to room temperature and quenched with MTBE (200 mL). The resulting mixture was filtered and the filter cake was collected, stirred in refluxing MTBE (100 mL) for 2 h before it was cooled and filtered. The filter cake was collected and dried in vacuo. The intermediate S145 was obtained as a yellow solid (2.09 g, 15%). HRMS (ESI): calcd for C₃₇H₄₀FN₂O₅⁺ [M+H]⁺ 611.2916, found 611.2917.

To a stirring solution of S145 (2.00 g, 3.090 mmol) in CH₂Cl₂ (50 mL) at 0° C. were added Et₃N (1.25 g, 12.361 mmol) and MsCl (708 mg, 6.181 mmol) in sequence. The resulting mixture stirred at 0° C. for 1 h before it was quenched with NaHCO₃ (sat. aq., 10 mL). The layers were separated, and the aqueous layer was extracted with CH₂Cl₂ (10 mL×3). The combined organic layers were dried (Na₂SO₄) and concentrated in vacuo. The intermediate S146 was obtained as a yellow solid (1.74 g, 81%) after flash column chromatography purification (silica gel, hexane:EtOAc=1:1). HRMS (ESI): calcd for C₃₈H₄₂FN₂O₇S⁺ [M+H]⁺ 689.2691, found 689.2693.

### Step 3:

To a stirring solution of S146 (1.50 g, 2.178 mmol) in DMF (15 mL) at room temperature was added piperidine (1.5 mL). The resulting mixture was stirred at room temperature for 30 min before it was concentrated in vacuo directly. The intermediate S147 was obtained as a yellow solid (980 mg, 87%) after RP-HPLC purification and lyophilization. HRMS (ESI): calcd for C₂₃H₃₂FN₂O₅S⁺ [M+H]⁺ 467.2010, found 467.2012.

### Step 4:

To a stirring solution of S147 (890 mg, 1.908 mmol) and 27 (503 mg, 1.908 mmol) in toluene (20 mL) at room temperature was added PPTS (96 mg, 0.382 mmol). The resulting mixture was heated to reflux and stirred overnight before it was cooled to room temperature and filtered. The filter cake was collected and stirred in refluxing toluene for 3 h before it was cooled and filtered. The filter cake was collected and dried in vacuo. The intermediate S148 was obtained as a white solid (650 mg, 49%) after RP-HPLC purification and lyophilization. HRMS (ESI): calcd for C₃₆H₄₁FN₃O₈S⁺ [M+H]⁺ 694.2593, found 694.2600.

### Step 5:

To a stirring solution of S148 (650 mg, 0.619 mmol) in MeOH (10 mL) at room temperature was added Pd/C (65 mg, 10% wt/wt). The resulting mixture was stirred at room temperature under H₂ atmosphere for 6 h before it was filtered through a pad of celite. The filtrate was collected and concentrated in vacuo. The intermediate S149 was obtained as a yellow solid (535 mg, 95%) after flash column chromatography purification (silica gel, CH₂Cl₂:hexane=10:1). HRMS (ESI): calcd for C₂₉H₃₅FN₃O₈S⁺ [M+H]⁺ 604.2123, found 604.2130.

### Step 6:

To a stirring solution of S149 (500 mg, 0.828 mmol) and S₁₁₉ (576 mg, 0.828 mmol) in acetone (10 mL) at room temperature were added K₂CO₃ (573 mg, 4.141 mmol) and KI (138 mg, 4.141 mmol) in sequence. The resulting mixture was heated to 60° C. and stirred for 20 h before it was cooled to room temperature and concentrated in vacuo directly. The intermediate S150 was obtained as a yellow solid (230 mg, 42%) after flash column chromatography purification (silica gel, DCM:MeOH=10:1). HRMS (ESI): calcd for C₃₂H₄₁FN₃O₉S⁺ [M+H]⁺ 662.2542, found 662.2546.

### Step 7:

To a stirring solution of S150 (230 mg, 0.348 mmol) in CH₂Cl₂ (4 mL) at room temperature was added TFA (2 mL). The resulting mixture was stirred at room temperature for 1 h before it was concentrated in vacuo directly. The product intermediate S151 was obtained as a yellow solid (152 mg, 72%) after RP-HPLC purification and lyophilization. HRMS (ESI): calcd for C₂₈H₃₃FN₃O₉S⁺ [M+H]⁺ 606.1916, found 606.1918.

### Step 8:

To a stirring solution of S151 (152 mg, 0.251 mmol) and 85 (228 mg, 0.753 mmol) in DMF (5 mL) at room temperature was added p-TsOH·H₂O (24 mg, 0.125 mmol). The resulting mixture was heated to 60° C. and stirred for 2 h before it was cooled to room temperature and concentrated in vacuo directly. The intermediate S152 was obtained as a white solid (89 mg, 38%) after RP-HPLC purification and lyophilization. HRMS (ESI): calcd for C₄₇H₅₁FN₅O₁₂S⁺ [M+H]⁺ 928.3233, found 928.3237.

### Step 9:

To a stirring solution of S152 (150 mg, 0.162 mmol) in DMF (2 mL) at room temperature was added piperidine (0.2 mL). The resulting mixture was stirred at room temperature for 30 min before it was concentrated in vacuo directly. The intermediate S153 was obtained as a yellow oil (74 mg, 65%) after RP-HPLC purification and lyophilization. HRMS (ESI): calcd for C₃₂H₄₁FN₅O₁₀S⁺ [M+H] 706.2553, found 706.2555.

### Step 10:

To a stirring solution of S21 (135 mg, 0.105 mmol) in DMF (10 mL) at room temperature was added HATU (40 mg, 0.105 mmol). The resulting mixture was stirred at room temperature for 15 min before addition of S153 (74 mg, 0.105 mmol) and DIPEA (41 mg, 0.315 mmol) in sequence. The resulting mixture was stirred at room temperature for further 1 h before it was concentrated in vacuo directly. The intermediate S154 was obtained as a dark yellow oil (207 mg, 100%) and used without further purification. HRMS (ESI): calcd for C₉₅H₁₃₈FN₁₄O₂₈S⁺ [M+H]⁺ 1973.9504, found 1973.9505.

### Step 11:

To a stirring solution of S154 (207 mg, 0.105 mmol) in CH₂Cl₂ (4 mL) at room temperature was added TFA (2 mL). The resulting mixture was stirred at room temperature for 1 h before it was concentrated in vacuo directly. The product A027 was obtained as a yellow solid (99 mg, 52% over 2 steps) after RP-HPLC purification and lyophilization. HRMS (ESI): calcd for C₈₃H₁₁₄FN₁₄O₂₈S⁺ [M+H]⁺ 1805.7626, found 1805.7630.

### Example 365: Synthesis of A024

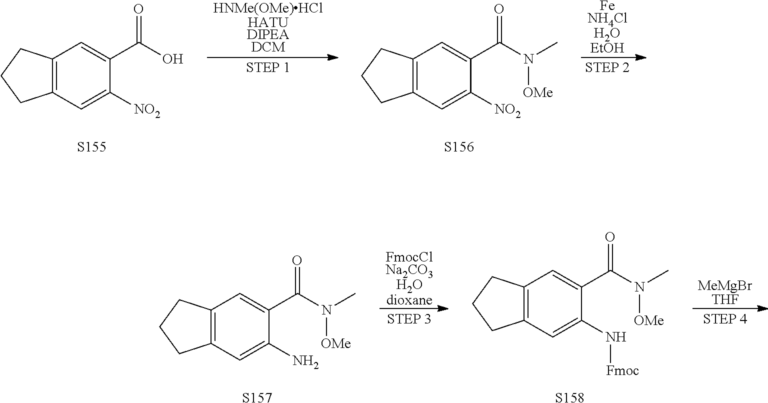

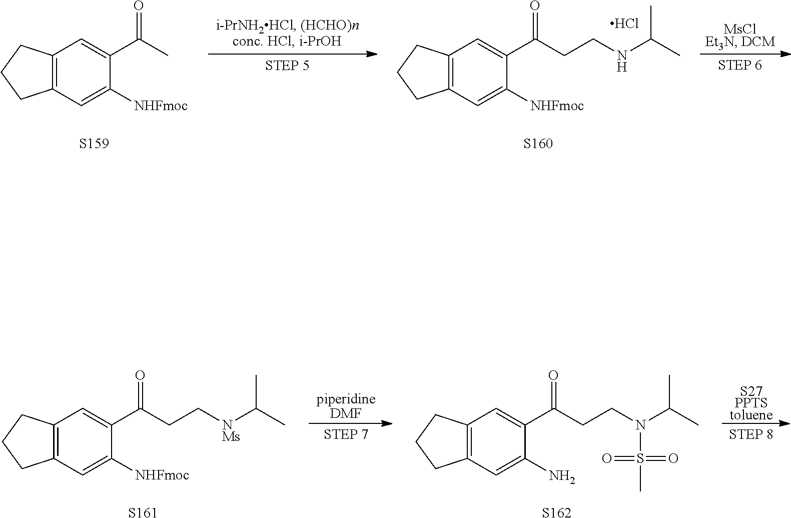

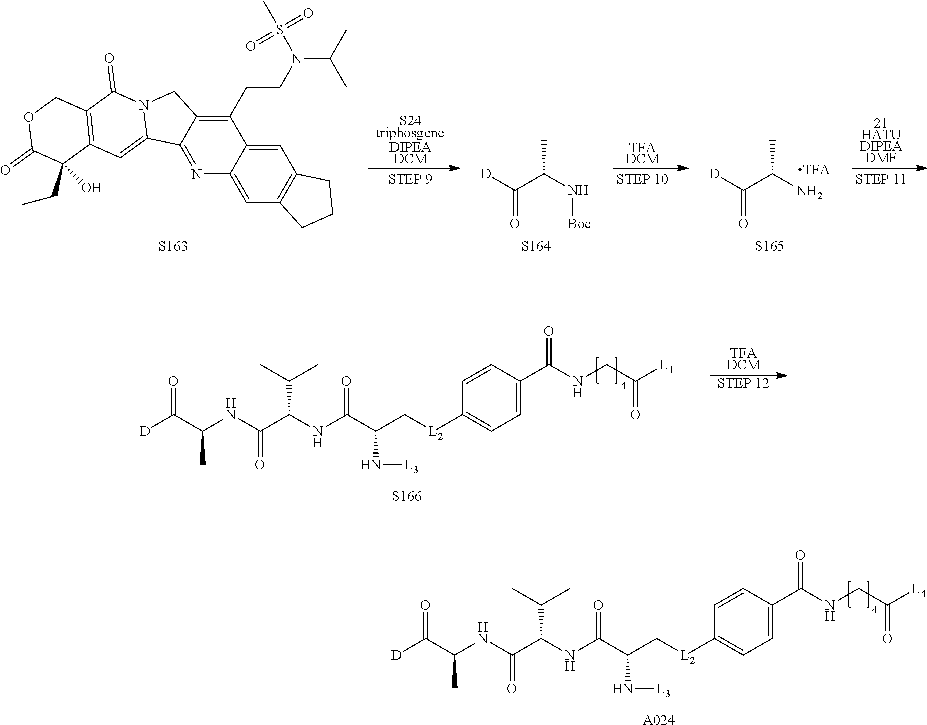

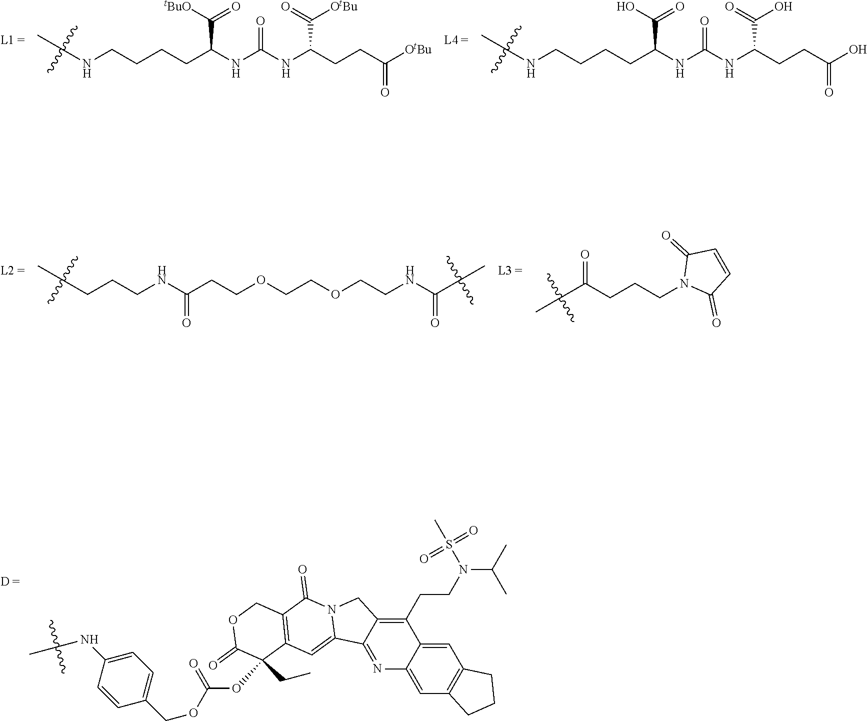 [SMILES: *NCCCC[C@H](NC(=O)N[C@@H](CCC(=O)OC(C)(C)C)C(=O)OC(C)(C)C)C(=O)OC(C)(C)C ; *NCCCC[C@H](NC(=O)N[C@@H](CCC(=O)O)C(=O)O)C(=O)O ; *CCCNC(=O)CCOCCOCCNC(*)=O ; *C(=O)CCCN1C(=O)C=CC1=O ; *Nc1ccc(COC(=O)O[C@]2(CC)C(=O)OCc3c2cc2n(c3=O)Cc3c-2nc2cc4c(cc2c3CCN(C(C)C)S(C)(=O)=O)CCC4)cc1]

### Step 1:

To a stirring solution of S155 (20.00 g, 96.530 mmol) and HNMe (OMe)·HCl (11.30 g, 115.836 mmol) in CH₂Cl₂ (250 mL) at room temperature were added HATU (44.05 g, 115.836 mmol) and DIPEA (37.43 g, 289.589 mmol). The resulting mixture was stirred at room temperature for 1 h before it was quenched with Na₂CO₃ (5% wt/wt aq., 100 mL). The layers were separated, and the organic layer was washed with HCl (1 M aq., 50 mL) and water (100 mL). The organic layers were dried (Na₂SO₄) and concentrated in vacuo. The intermediate S156 was obtained as a yellow solid (22.00 g, 91%) after flash column chromatography purification (silica gel, hexane:EtOAc=2:1). HRMS (ESI): calcd for C₁₂H₁₅N2O₄⁺ [M+H]⁺ 251.1026, found 251.1033.

To a stirring solution of S156 (20.00 g, 79.920 mmol) in EtOH/H₂O (300 mL, 50% V/V) at room temperature were added NH₄Cl (42.75 g, 799.200 mmol) and Fe powder (31.24 g, 159.840 mmol). The resulting mixture was heated to 80° C. and stirred for 2 h before it was cooled to room temperature and filtered through a pad of celite. The filtrate was collected and concentrated in vacuo. The intermediate S₁₅₇ was obtained as a yellow solid (13.40 g, 71%) after flash column chromatography purification (silica gel, hexane:EtOAc=2:1). HRMS (ESI): calcd for C₁₂H₁₇N2O₂⁺ [M+H]⁺ 221.1285, found 221.1290.

### Step 3:

To a stirring solution of S157 (13.00 g, 59.018 mmol) in 1,4-dioxane/H₂O (500 mL, 70% V/V) at 0° C. were added Na₂CO₃ (18.77 g, 177.092 mmol) and FmocCl (38.17 g, 147.545 mmol). The resulting mixture was warmed to room temperature and stirred for 12 h before it was concentrated in vacuo. The residual aqueous layer was extracted with EtOAc (200 mL×3). The combined organic layers were dried (Na₂SO₄) and concentrated in vacuo. The intermediate S158 was obtained as a white solid (25.12 g, 96%) after flash column chromatography purification (silica gel, hexane:EtOAc=4:1). HRMS (ESI): calcd for C₂₇H₂₇N2O₄⁺ [M+H]⁺ 443.1965, found 443.1966.

Step 4: To a stirring solution of S158 (20.00 g, 45.196 mmol) in THF (300 mL) at 0° C. was added MeMgBr (22.60 mL, 3.0 mol/L in Et₂O, 67.794 mmol). The resulting mixture was warmed to room temperature and stirred for 1 h before it was quenched with NH₄Cl (sat. aq., 100 mL). The layers were separated, and the aqueous layer was extracted with EtOAc (100 mL×3). The combined organic layers were dried (Na₂SO₄) and concentrated in vacuo. The intermediate S159 was obtained as a yellow solid (19.52 g, 86%) after flash column chromatography purification (silica gel, hexane:EtOAc=5:1). HRMS (ESI): calcd for C₂₆H₂₄NO₃⁺ [M+H]⁺ 398.1751, found 398.1760.

### Step 5:

To a stirring solution of S159 (15.00 g, 37.739 mmol) and i-Pr₂NH·HCl (18.03 g, 188.693 mmol) and (HCHO) n (5.67 g, 188.693 mmol) in i-PrOH (300 mL) at room temperature was added conc. HCl (3 mL). The resulting mixture was heated to reflux and stirred for 12 h before it was cooled to room temperature and quenched with MTBE (500 mL). The resulting mixture was filtered and the filter cake was collected, stirred in refluxing MTBE (50 mL) for 2 h before it was cooled and filtered. The filter cake was collected and dried in vacuo. The intermediate S160 was obtained as a yellow solid (9.85 g, 51%) after RP-HPLC purification and lyophilization. HRMS (ESI): calcd for C₃₀H₃₃N2O₃⁺ [M+H]⁺ 469.2486, found 469.2486.

### Step 6:

To a stirring solution of S160 (9.00 g, 17.820 mmol) in CH₂Cl₂ (150 mL) at 0° C. were added Et₃N (7.21 g, 71.279 mmol) and MsCl (4.08 mg, 35.639 mmol) in sequence. The resulting mixture stirred at 0° C. for 1 h before it was quenched with NaHCO₃ (sat. aq., 20 mL). The layers were separated, and the aqueous layer was extracted with CH₂Cl₂ (30 mL×3). The combined organic layers were dried (Na₂SO₄) and concentrated in vacuo. The intermediate S161 was obtained as a yellow solid (8.33 g, 85%) after flash column chromatography purification (silica gel, hexane:EtOAc=1:1). HRMS (ESI): calcd for C₃₁H₃₅N₂O₅S⁺ [M+H]⁺ 547.2261, found 547.2263.

### Step 7:

To a stirring solution of S161 (8.00 g, 14.634 mmol) in DMF (100 mL) at room temperature was added piperidine (10 mL). The resulting mixture was stirred at room temperature for 30 min before it was concentrated in vacuo directly. The intermediate S162 was obtained as a yellow solid (3.78 g, 79%) after flash column chromatography purification (silica gel, CH₂Cl₂:hexane=10:1). HRMS (ESI): calcd for C₁₆H₂₅N2O₃S⁺ [M+H]⁺ 325.1580, found 325.1581.

### Step 8:

To a stirring solution of S162 (3.50 g, 10.788 mmol) and S27 (3.41 g, 12.945 mmol) in toluene (200 mL) at room temperature was added PPTS (1.36 g, 5.394 mmol). The resulting mixture was heated to reflux and stirred overnight before it was cooled to room temperature and filtered. The filter cake was collected and stirred in refluxing toluene for 3 h before it was cooled and filtered. The filter cake was collected and dried in vacuo. The intermediate S163 was obtained as a white solid (1.60 g, 26%) after RP-HPLC purification and lyophilization. HRMS (ESI): calcd for C₂₉H₃₄N₃O₆S⁺ [M+H]⁺ 552.2163, found 552.2166.

### Step 9:

To a stirring solution of S163 (1.50 g, 2.719 mmol) in CH₂Cl₂ (50 mL) at room temperature were added triphosgene (275 mg, 0.924 mmol) The resulting mixture was stirred at room temperature for 2 h before addition of S24 (881 mg, 2.991 mmol) and DIPEA (1.05 g, 8.157 mmol) in sequence. The resulting mixture was stirred at room temperature for further 2 h before it was quenched with NaHCO₃ (sat. aq., 10 mL). The layers were separated, and the aqueous layer was extracted with CH₂C₁₂ (10 mL×3). The combined organic layers were dried (Na₂SO₄) and concentrated in vacuo. The intermediate S₁₆₄ was obtained as a yellow solid (1.02 g, 43%) after flash column chromatography purification (CH₂Cl₂:hexane=20:1). HRMS (ESI): calcd for C₄₅H₅₄N5OnS⁺ [M+H]⁺ 872.3535, found 872.3539.

### Step 10:

To a stirring solution of S164 (200 mg, 0.229 mmol) in CH₂Cl₂ (4 mL) at room temperature was added TFA (2 mL). The resulting mixture was stirred at room temperature for 1 h before it was concentrated in vacuo directly. The intermediate S165 was obtained as a yellow solid (130 mg, 64%) after RP-HPLC purification and lyophilization. HRMS (ESI): calcd for C₄₀H₄₆N5O₉S⁺ [M+H]⁺ 772.3011, found 772.3012.

### Step 11:

To a stirring solution of S21 (189 mg, 0.147 mmol) in DMF (10 mL) at room temperature was added HATU (56 mg, 0.147 mmol). The resulting mixture was stirred at room temperature for 15 min before addition of S165 (130 mg, 0.147 mmol) and DIPEA (57 mg, 0.440 mmol) in sequence. The resulting mixture was stirred at room temperature for further 1 h before it was concentrated in vacuo directly. The intermediate S166 was obtained as a dark yellow oil (299 mg, 100%) and used without further purification. HRMS (ESI): calcd for C₁₀₃H₁₄₃N₁₄O₂₇S⁺ [M+H]⁺ 2039.9962, found 2039.9969.

Step 12:

To a stirring solution of S166 (299 mg, 0.147 mmol) in CH₂Cl₂ (4 mL) at room temperature was added TFA (2 mL). The resulting mixture was stirred at room temperature for 1 h before it was concentrated in vacuo directly. The product A024 was obtained as a yellow solid (100 mg, 36% over 2 steps) after RP-HPLC purification and lyophilization. HRMS (ESI): calcd for C₉₁H₁₁₉N₁₄O₂₇S⁺ [M+H]⁺ 1871.8084, found 1871.8088.

### Example 366: Synthesis of A025

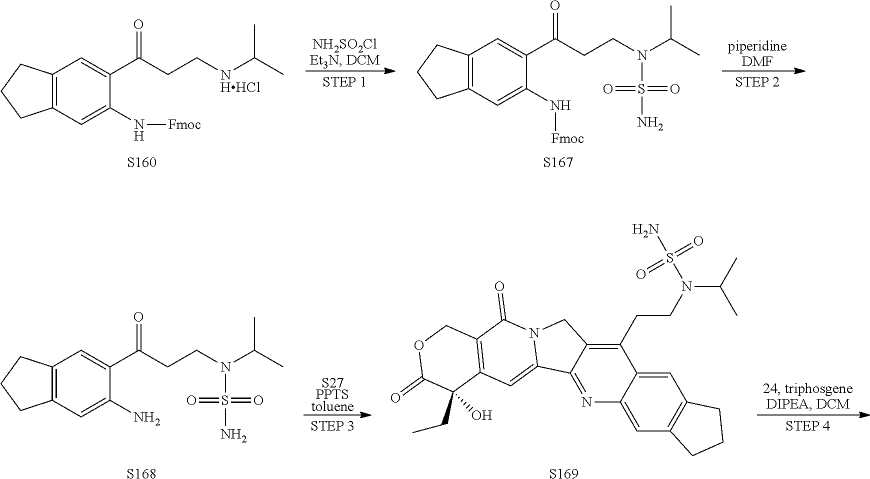

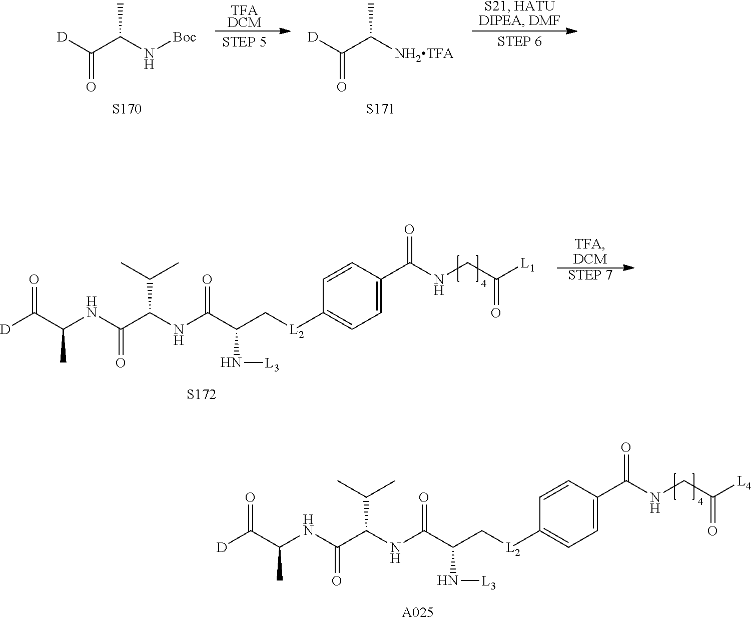

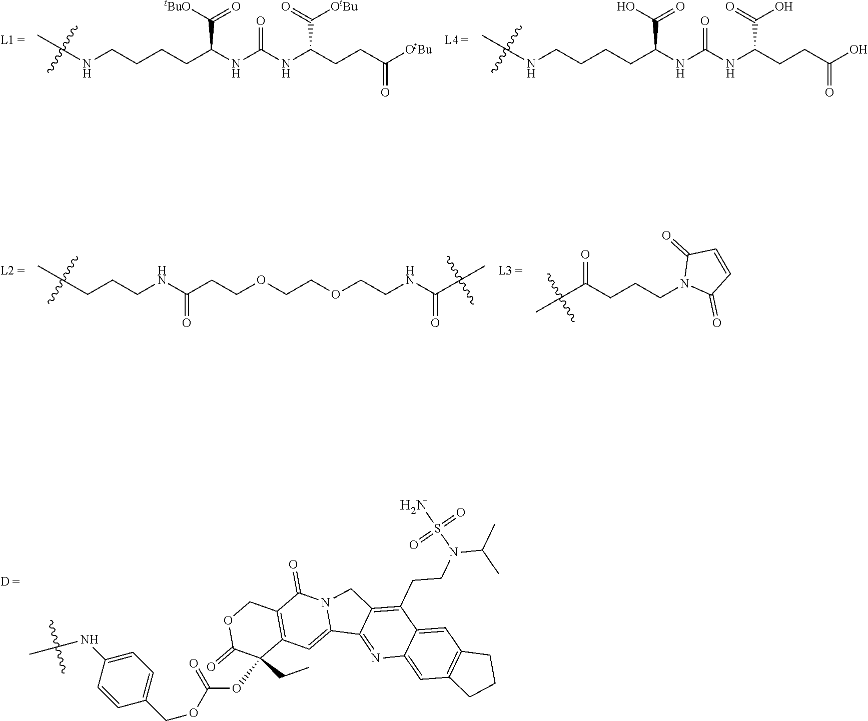 [SMILES: *NCCCC[C@H](NC(=O)N[C@@H](CCC(=O)OC(C)(C)C)C(=O)OC(C)(C)C)C(=O)OC(C)(C)C ; *NCCCC[C@H](NC(=O)N[C@@H](CCC(=O)O)C(=O)O)C(=O)O ; *CCCNC(=O)CCOCCOCCNC(*)=O ; *C(=O)CCCN1C(=O)C=CC1=O ; *Nc1ccc(COC(=O)O[C@]2(CC)C(=O)OCc3c2cc2n(c3=O)Cc3c-2nc2cc4c(cc2c3CCN(C(C)C)S(N)(=O)=O)CCC4)cc1]

Step 1˜STEP 7: Intermediate S₁₆₇˜S₁₇₂ and product A025 were prepared according to the procedures in A024. A025: HRMS (ESI): calcd for C₉₀H₁₁₈N₁₅O₂₇S⁺ [M+H]⁺ 1872.8037, found 1872.8037.

### Example 367: Synthesis of A063

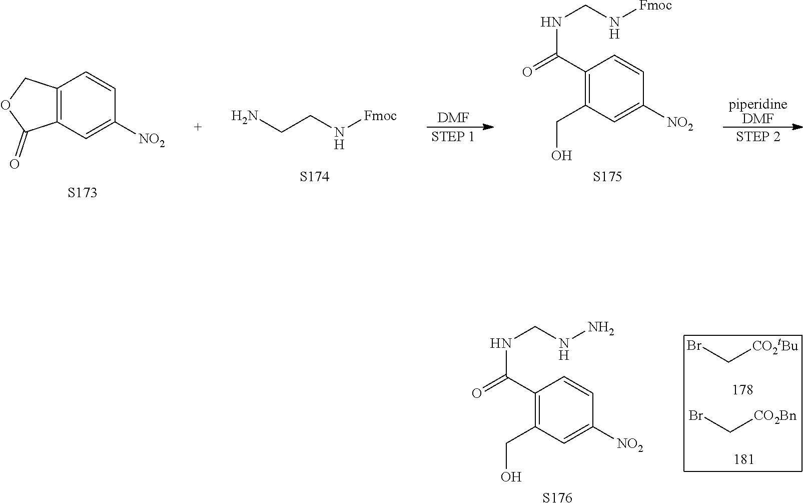

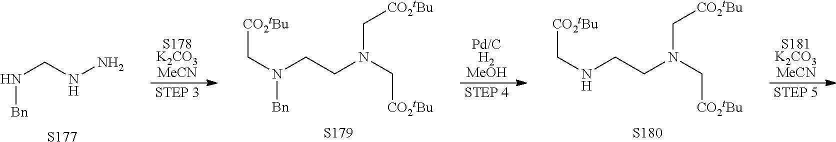

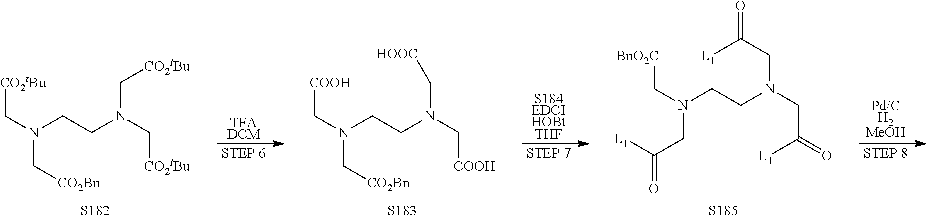

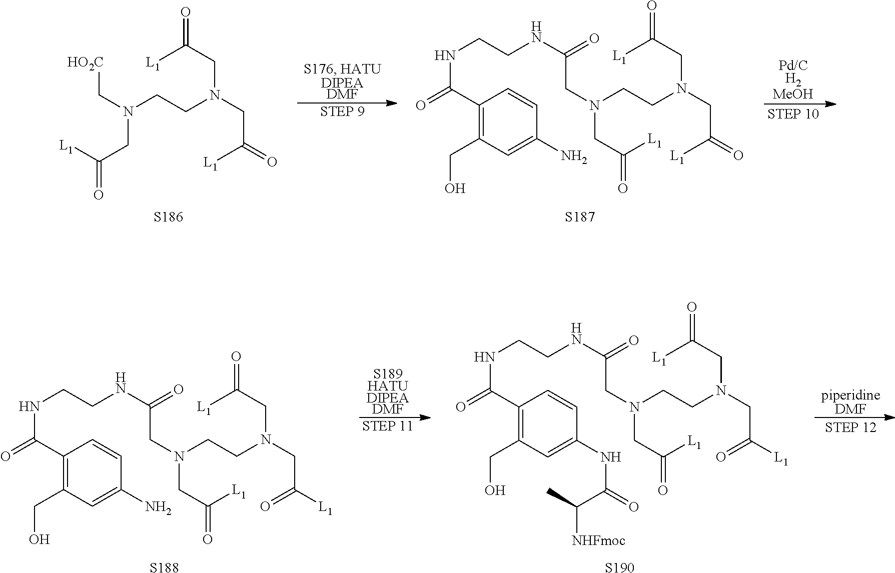

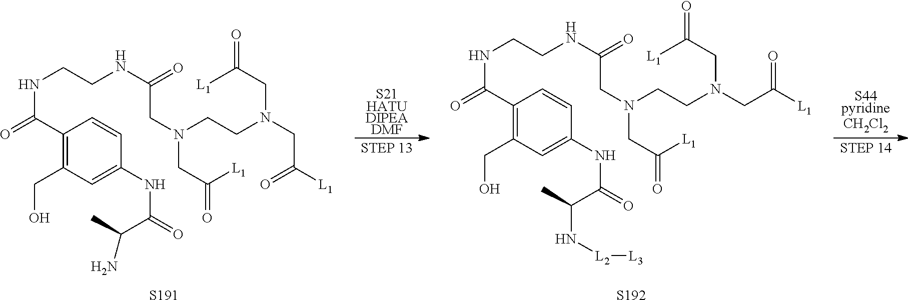

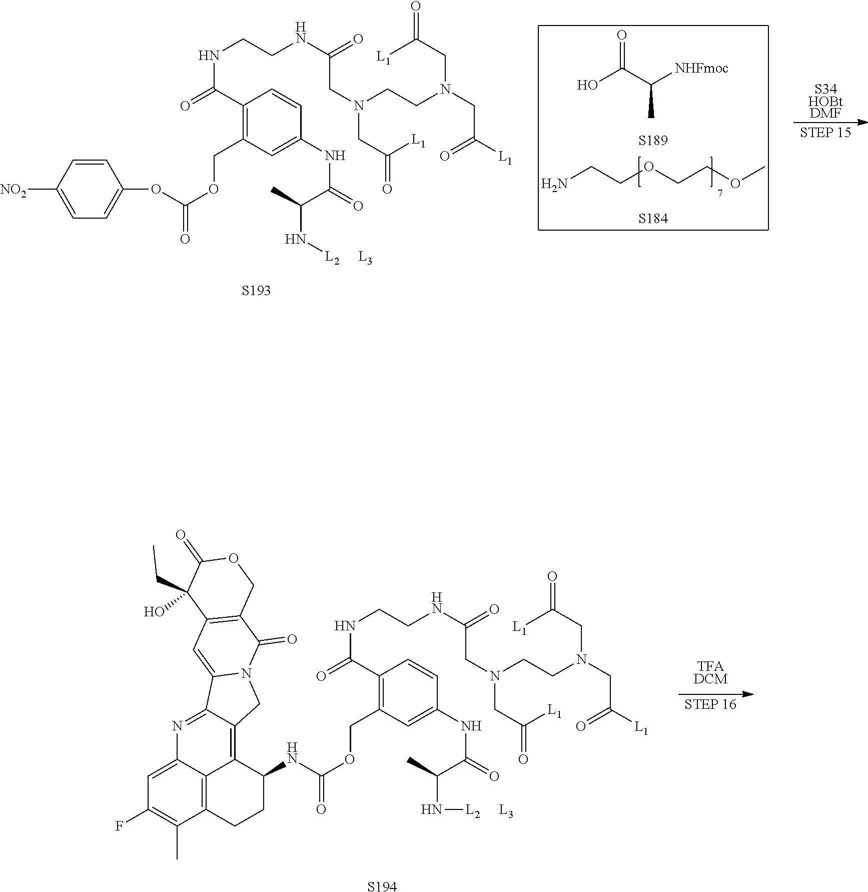

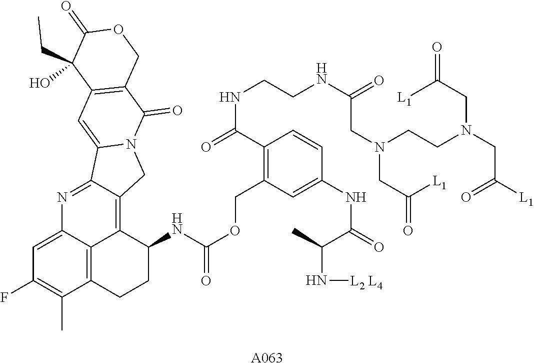

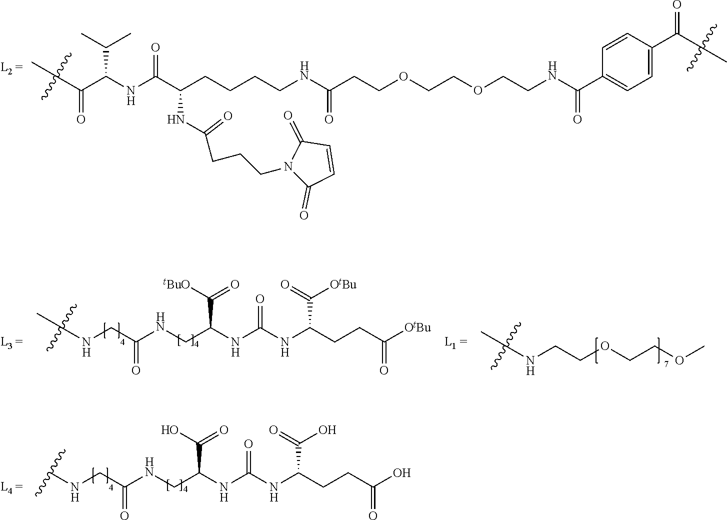

### Step 1:

To a stirring solution of S173 (10.00, 55.825 mmol) in DMF (100 mL) at room temperature was added S174 (16.55 g, 58.617 mmol). The resulting mixture was stirred at room temperature for 24 h before it was concentrated in vacuo directly. The intermediate S175 was obtained as a yellow oil (22.22 g, 86%) after flash column chromatography purification (hexane:EtOAc=2:1). HRMS (ESI): calcd for C₂₅H₂₄N3O₆⁺ [M+H]⁺ 462.1660, found 462.1661.

### Step 2:

To a stirring solution of S175 (20.00 g, 43.340 mmol) in DMF (200 mL) at room temperature was added piperidine (20 mL). The resulting mixture was stirred at room temperature for 30 min before it was concentrated in vacuo directly. The intermediate S176 was obtained as a yellow solid (9.66 g, 93%) after RP-HPLC purification and lyophilization. HRMS (ESI): calcd for C₁₀H₁₄N3O₄⁺ [M+H]⁺ 240.0979, found 240.0980.

### Step 3:

To a stirring solution of S177 (10.00 g, 66.565 mmol) and S178 (58.43 g, 299.541 mmol) in MeCN (300 mL) at room temperature were added K₂CO₃ (41.40 g, 299.541 mmol). The resulting mixture was heated to 60° C. and stirred for 24 h before it was cooled to room temperature and concentrated in vacuo directly. The intermediate S179 was obtained as a yellow oil (27.45 g, 84%) after flash column chromatography purification (silica gel, hexane:EtOAc=5:1). HRMS (ESI): calcd for C₂₇H₄₅N2O₆⁺ [M+H]⁺ 493.3272, found 493.3272.

### Step 4:

To a stirring solution of S179 (25.00 g, 50.745 mmol) in MeOH (500 mL) at room temperature was added Pd/C (2.50 g, 10% wt/wt). The resulting mixture was stirred at room temperature under H₂ atmosphere for 2 h before it was filtered through a pad of celite. The filtrate was collected and concentrated in vacuo. The intermediate S180 was obtained as a yellow oil (20.42 g, 100%) and used without further purification. HRMS (ESI): calcd for C₂₀H₃₉N2O₆⁺ [M+H]⁺ 403.2803, found 403.2803.

### Step 5:

To a stirring solution of S180 (20.42 g, 50.729 mmol) and S181 (17.43 g, 76.094 mmol) in MeCN (300 mL) at room temperature were added K₂CO₃ (10.52 g, 76.094 mmol). The resulting mixture was heated to 60° C. and stirred for 24 h before it was cooled to room temperature and concentrated in vacuo directly. The intermediate S182 was obtained as a yellow oil (25.66 g, 92%) after flash column chromatography purification (silica gel, hexane:EtOAc=4:1). HRMS (ESI): calcd for C₂₉H₄₇N2O₈⁺ [M+H]⁺ 551.3327, found 551.3328.

### Step 6:

To a stirring solution of S182 (23.66 g, 42.964 mmol) in CH₂Cl₂ (150 mL) at room temperature was added TFA (300 mL). The resulting mixture was stirred at room temperature for 2 h before it was concentrated in vacuo directly. The residue was dispersed with MTBE (250 mL) and stirred for 30 min before it was filtered. The filter cake was collected and dried in vacuo. The intermediate S183 was obtained as a yellow solid (13.00 g, 79%) and used without further purification. HRMS (ESI): calcd for C₁₇H₂₃N2O₈⁺ [M+H]⁺ 383.1449, found 383.1452.

### Step 7:

To a stirring solution of S183 (10.00 g, 26.153 mmol) and S184 (33.10 g, 86.304 mmol) in THF (500 mL) at room temperature were added HOBt (11.66 g, 86.304 mmol) and EDCI·HCl (16.54 g, 86.304 mmol) in sequence. The resulting mixture was stirred at room temperature for 5 h before it was quenched with HCl (1.0 M, aq., 200 mL). The layers were separated, and the aqueous layer was extracted with EtOAc (100 mL×3). The combined organic layers were dried (Na₂SO₄) and concentrated in vacuo. The intermediate S185 was obtained as a yellow oil (27.85 g, 72%) after flash column chromatography purification (CH₂Cl₂:MeOH=10:1). HRMS (ESI): calcd for C₆₈H₁₂₈N5O₂₉⁺ [M+H]⁺ 1478.8689, found 1478.8693.

### Step 8:

To a stirring solution of S185 (5.00 g, 3.38 mmol) in MeOH (100 mL) at room temperature was added Pd/C (0.50 g, 10% wt/wt). The resulting mixture was stirred at room temperature under H₂ atmosphere for 2 h before it was filtered through a pad of celite. The filtrate was collected and concentrated in vacuo. The intermediate S186 was obtained as a yellow solid (4.70 g, 100%) and used without further purification. HRMS (ESI): calcd for C₆₁H₁₂₂N5O₂₉⁺ [M+H]⁺ 1388.8220, found 1388.8225.

### Step 9:

To a stirring solution of S186 (4.70 g, 3.385 mmol) in DMF (100 mL) at room temperature was added HATU (1.42 g, 3.723 mmol). The resulting mixture was stirred at room temperature for 15 min before addition of S176 (891 mg, 3.723 mmol) and DIPEA (1.31 g, 10.154 mmol) in sequence. The resulting mixture was stirred at room temperature for further 1 h before it was concentrated in vacuo directly. The intermediate S187 was obtained as a yellow solid (4.11 g, 75%) after RP-HPLC purification and lyophilization. HRMS (ESI): calcd for C₇₁H₁₃₃N₈O₃₂⁺ [M+H]⁺ 1609.9020, found 1609.9022.

### Step 10:

To a stirring solution of S187 (1.00 g, 0.621 mmol) in MeOH (20 mL) at room temperature was added Pd/C (0.10 g, 10% wt/wt). The resulting mixture was stirred at room temperature under H₂ atmosphere for 5 h before it was filtered through a pad of celite. The filtrate was collected and concentrated in vacuo. The intermediate S188 was obtained as a yellow solid (980 mg, 100%) and used without further purification. HRMS (ESI): calcd for C₇₁H₁₃₅N₈O₃₀⁺ [M+H]⁺ 1579.9279, found 1579.9284.

### Step 11:

To a stirring solution of S189 (232 mg, 0.744 mmol) in DMF (10 mL) at room temperature were added HATU (283 mg, 0.744 mmol) and DIPEA (120 mg, 0.930 mmol). The resulting mixture was stirred at room temperature for 15 min before addition of S188 (980 mg, 0.620 mmol). The resulting mixture was stirred at room temperature for further 1 h before it was concentrated in vacuo directly. The intermediate S190 was obtained as a yellow solid (800 mg, 69%) after RP-HPLC purification and lyophilization. HRMS (ESI): calcd for C₈₉H₁₅₀N₉O₃₃⁺ [M+H]⁺ 1873.0331, found 1873.0333.

### Step 12:

To a stirring solution of S190 (800 mg, 0.427 mmol) in DMF (10 mL) at room temperature was added piperidine (1 mL). The resulting mixture was stirred at room temperature for 30 min before it was concentrated in vacuo directly. The intermediate S191 was obtained as a yellow solid (520 mg, 74%) after RP-HPLC purification and lyophilization. HRMS (ESI): calcd for C₇₄H₁₄₀N₉O₃₁⁺ [M+H]⁺ 1650.9650, found 1650.9658.

### Step 13:

To a stirring solution of S21 (446 mg, 0.346 mmol) in DMF (10 mL) at room temperature was added HATU (132 mg, 0.346 mmol). The resulting mixture was stirred at room temperature for 15 min before addition of S191 (520 mg, 0.315 mmol) and DIPEA (122 mg, 0.945 mmol) in sequence. The resulting mixture was stirred at room temperature for further 1 h before it was concentrated in vacuo directly. The intermediate S192 was obtained as a yellow solid (600 mg, 65%) after RP-HPLC purification and lyophilization. HRMS (ESI): calcd for C₁₃₇H₂₃₇N₁₈O₄₉⁺ [M+H]⁺ 2918.6601, found 2918.6605.

### Step 14:

To a stirring solution of S192 (500 mg, 0.171 mmol) in CH₂Cl₂ (10 mL) at 0° C. were added pyridine (28 mg, 0.343 mmol) and S44 (52 mg, 0.257 mmol) in sequence. The resulting mixture was stirred at 0° C. for 2 h before it was quenched with NaHCO₃ (sat. aq., 5 mL). The layers were separated, and the aqueous layer was extracted with CH₂Cl₂ (10 mL×3). The combined organic layers were dried (Na₂SO₄) and concentrated in vacuo. The intermediate S193 was obtained as a yellow oil (425 mg, 80%) after RP-HPLC purification and lyophilization. HRMS (ESI): calcd for C₁₄₄H₂₄₀N19O₅₃⁺ [M+H] 3083.6663, found 3083.6666.

### Step 15:

To a stirring solution of S193 (400 mg, 0.130 mmol) in DMF (10 mL) at room temperature were added HOBt (21 mg, 0.156 mmol) and S34 (83 mg, 0.156 mmol). The resulting mixture was stirred at room temperature for 6 h before it was concentrated in vacuo directly. The intermediate S194 was obtained as a dark yellow oil (428 mg, 100%) and used without further purification. HRMS (ESI): calcd for C₁₆₂H₂₅₇FN₂₁O₅₄⁺ [M+H]⁺ 3379.7988, found 3379.7988.

### Step 16:

To a stirring solution of S194 (428 mg, 0.127 mmol) in CH₂Cl₂ (5 mL) at room temperature was added TFA (2.5 mL). The resulting mixture was stirred at room temperature for 1 h before it was concentrated in vacuo directly. The product A063 was obtained as a yellow solid (125 mg, 31% over 2 steps) after RP-HPLC purification and lyophilization. HRMS (ESI): calcd for C₁₅₀H₂₃₃FN₂₁O₅₄⁺ [M+H]⁺ 3211.6110, found 3211.6111.

### Example 368: Synthesis of A037

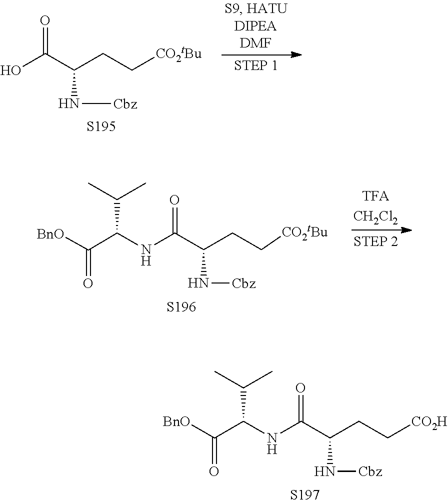 [SMILES: CC(C)(C)OC(=O)CC[C@H](NC(=O)OCc1ccccc1)C(=O)O ; "S195" ; CC(C)[C@H](NC(=O)[C@H](CCC(=O)OC(C)(C)C)NC(=O)OCc1ccccc1)C(=O)OCc1ccccc1 ; "S196" ; CC(C)[C@H](NC(=O)[C@H](CCC(=O)O)NC(=O)OCc1ccccc1)C(=O)OCc1ccccc1 ; "S197"]

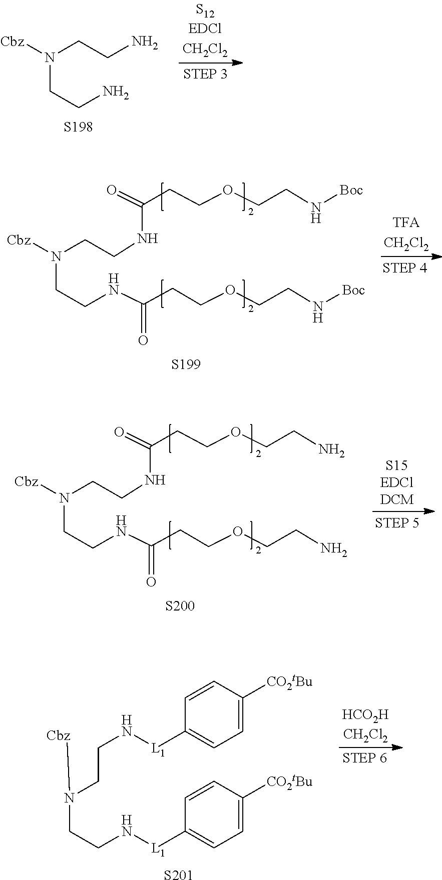

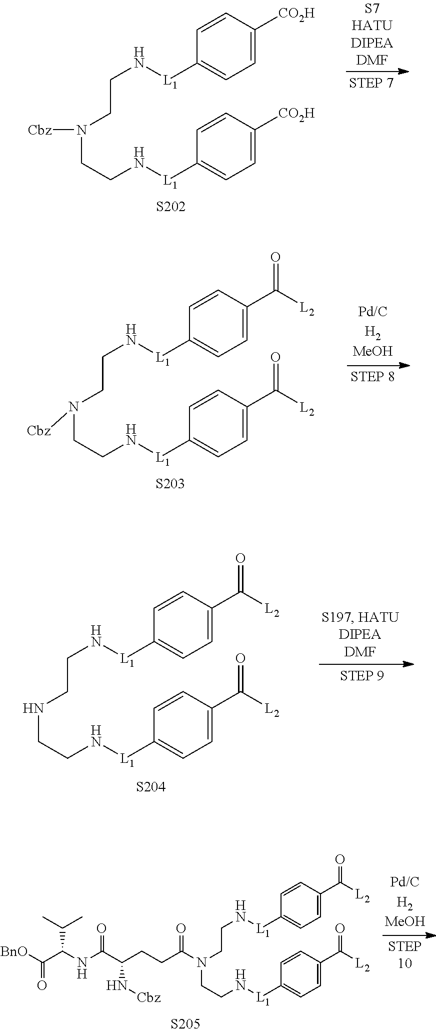

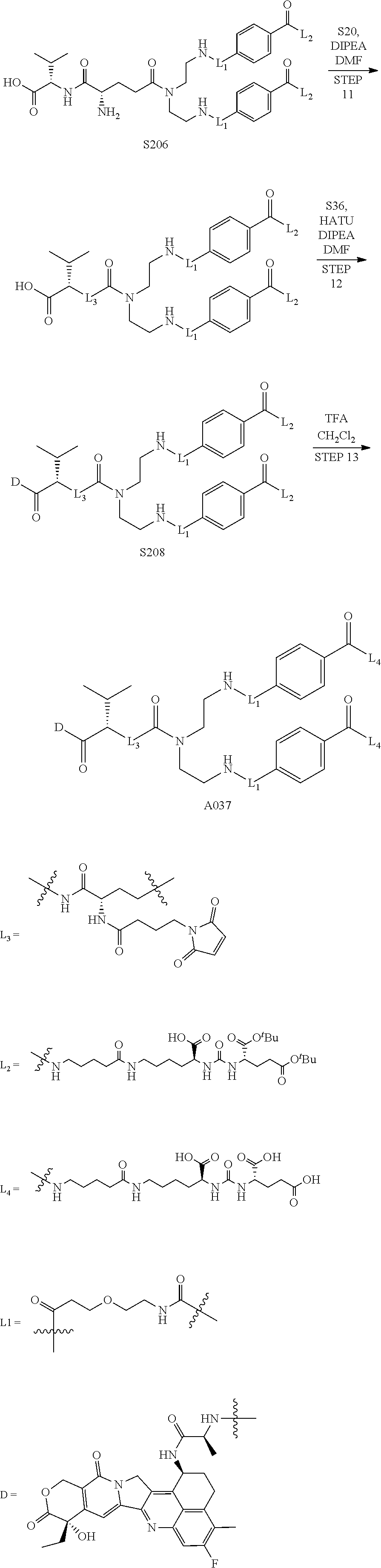

### Step 1:

To a stirring solution of S195 (5.00 g, 14.821 mmol) in CH₂Cl₂ (100 mL) at 0° C. were added HATU (5.92 g, 15.562 mmol), S₉ (3.79 g, 15.562 mmol) and DIPEA (4.79 g, 37.053 mmol) in sequence. The resulting mixture was warmed to room temperature and stirred for 2 h before it was quenched with citric acid (10% wt/wt, aq., 20 mL). The layers were separated, and the aqueous layer was washed with NaCl (sat. aq., 20 mL). The combined organic layers were dried (Na₂SO₄) and concentrated in vacuo. The intermediate S196 was obtained as a white solid (7.70 g, 98%) by recrystallization (hexane:CH₂Cl_{2=20:1}, room temperature). HRMS (ESI): calcd for C₂₉H₃₉N2O₇⁺ [M+H]⁺ 527.2752, found 527.2755.

### Step 2:

To a stirring solution of S196 (7.50 g, 14.241 mmol) in CH₂Cl₂ (100 mL) at room temperature was added TFA (33 mL). The resulting mixture was stirred at room temperature for 2 h before it was concentrated in vacuo directly. The intermediate S197 was obtained as a yellow oil (6.70 g, 100%) and used without further purification. HRMS (ESI): calcd for C₂₅H₃₁N2O₇⁺ [M+H]⁺ 471.2126, found 471.2131.

### Step 3:

To a stirring solution of S198 (5.00 g, 21.079 mmol) and S12 (12.85 g, 46.374 mmol) in CH₂Cl₂ (100 mL) at 0° C. was added EDCI (8.89 g, 46.374 mmol). The resulting mixture was warmed to room temperature and stirred for 2 h before it quenched with citric acid (10% wt/wt, aq., 20 mL). The layers were separated, and the aqueous layer was washed with NaCl (sat. aq., 20 mL). The combined organic layers were dried (Na₂SO₄) and concentrated in vacuo. The intermediate S199 was obtained as a white solid (15.20 g, 95%) by recrystallization (hexane:CH₂Cl₂=5:1, room temperature). HRMS (ESI): calcd for C₃₆H₆₂N5O₁₂⁺ [M+H]⁺ 756.4389, found 756.4396.

### Step 4:

To a stirring solution of S199 (7.50 g, 19.844 mmol) in CH₂Cl₂ (100 mL) at room temperature was added TFA (50 mL). The resulting mixture was stirred at room temperature for 2 h before it was concentrated in vacuo directly. The intermediate S200 was obtained as a yellow solid (14.60 g, 94%) with stirred with MTBE (100 mL). HRMS (ESI): calcd for C₂₆H₄₆N5O₈⁺ [M+H]⁺ 556.3341, found 556.3342.

### Step 5:

To a stirring solution of S200 (10.00 g, 12.760 mmol) and S15 (6.24 g, 28.071 mmol) in CH₂Cl₂ (150 mL) at 0° C. was added EDCI (5.38 g, 28.071 mmol). The resulting mixture was warmed to room temperature and stirred for 2 h before it quenched with citric acid (10% wt/wt, aq., 20 mL). The layers were separated, and the aqueous layer was washed with NaCl (sat. aq., 20 mL). The combined organic layers were dried (Na₂SO₄) and concentrated in vacuo. The intermediate S201 was obtained as a yellow oil (11.30 g, 92%) after flash column chromatography purification (CH₂Cl₂:MeOH=10:1). HRMS (ESI): calcd for C₅₀H₇₀N5O₁₄⁺ [M+H]⁺ 964.4914, found 964.4918.

### Step 6:

To a stirring solution of S201 (11.30 g, 11.721 mmol) in CH₂Cl₂ (100 mL) at 40° C. was added HCO₂H (200 mL). The resulting mixture was stirred at 40° C. for 12 h before it was concentrated in vacuo directly. The residue was dissolved with water (100 mL) and extracted with EtOAc (100 mL×3). The combined organic layers were dried (Na₂SO₄) and concentrated in vacuo. The intermediate S202 was obtained as a white solid (7.65 g, 76%) by recrystallization (hexane:EtOAc=1:1, room temperature). HRMS (ESI): calcd for C₄₂H₅₄N5O₁₄⁺ [M+H]⁺ 852.3662, found 852.3663.

### Step 7:

To a stirring solution of S202 (5.00 g, 5.869 mmol) in CH₂Cl₂ (150 mL) at room temperature were added HATU (4.69 g, 12.325 mmol) and DIPEA (2.28 g, 17.607 mmol) in sequence. The resulting mixture was stirred at room temperature for 0.5 h before addition of a solution of 7 (7.23 g, 12.325 mmol) in CH₂C₁₂ (80 mL). The resulting mixture was stirred at room temperature for further 1 h before it was quenched with HCl (1 M aq, 50 mL). The layers were separated and the aqueous layer was extracted with CH₂Cl₂ (3× 50 mL). The combined organic layers were washed with NaCl (sat. aq., 50 mL), dried (Na₂SO₄) and concentrated in vacuo. The intermediate S203 was obtained as a yellow oil (7.77 g, 67%) after flash column chromatography purification (silica gel, CH₂Cl₂:MeOH=10:1). HRMS (ESI): calcd for C₁₀₀H₁₅₈N₁₃O₂₈⁺ [M+H]⁺ 1989.1334, found 1989.1335.

### Step 8:

To a stirring solution of S203 (7.77 g, 3.906 mmol) in MeOH (150 mL) at room temperature was added Pd/C (0.77 g, 10% wt/wt). The resulting mixture was stirred at room temperature under H₂ atmosphere for 5 h before it was filtered through a pad of celite. The filtrate was collected and concentrated in vacuo. The intermediate S204 was obtained as a yellow solid (5.15 g, 71%) after RP-HPLC purification and lyophilization. HRMS (ESI): calcd for C₉₂H₁₅₂N₁₃O₂₆⁺ [M+H]⁺ 1855.0966, found 1855.0970.

### Step 9:

To a stirring solution of S197 (500 mg, 1.063 mmol) in DMF (10 mL) at room temperature was added HATU (425 mg, 1.116 mmol). The resulting mixture was stirred at room temperature for 15 min before addition of S204 (2.07 g, 1.116 mmol) in DMF (10 mL) and DIPEA (344 mg, 2.657 mmol) in sequence. The resulting mixture was stirred at room temperature for further 1 h before it was concentrated in vacuo directly. The intermediate S205 was obtained as a dark yellow oil (2.45 g, 100%) and used without further purification. HRMS (ESI): calcd for C₁₁₇H₁₈₀N₁₅O₃₂⁺ [M+H]⁺ 2307.2913, found 2307.2914.

### Step 10:

To a stirring solution of S204 (2.45 g, 1.062 mmol) in MeOH (100 mL) at room temperature was added Pd/C (245 mg, 10% wt/wt). The resulting mixture was stirred at room temperature under H₂ atmosphere for 2 h before it was filtered through a pad of celite. The filtrate was collected and concentrated in vacuo. The intermediate S205 was obtained as a dark yellow oil (2.21 g, 100%) and used without further purification. HRMS (ESI): calcd for C₁₀₂H₁₆₈N₁₅O₃₀⁺ [M+H]⁺ 2083.2076, found 2083.2077.

### Step 11:

To a stirring solution of S206 (2.21 g, 1.061 mmol) in DMF (50 L) at room temperature were added S20 (556 mg, 1.591 mmol) and DIPEA (275 mg, 2.121 mmol). The resulting mixture was stirred at room temperature for 3 h before it was concentrated in vacuo directly. The intermediate S207 was obtained as a light yellow solid (655 mg, 27% over 3 steps) after RP-HPLC purification and lyophilization. HRMS (ESI): calcd for C₁₁₀H₁₇₅N16O₃₃⁺ [M+H]⁺ 2248.2502, found 2248.2504.

### Step 12:

To a stirring solution of S207 (655 mg, 0.291 mmol) in DMF (10 mL) at room temperature was added HATU (116 mg, 0.306 mmol). The resulting mixture was stirred at room temperature for 15 min before addition of S36 (190 mg, 0.306 mmol) and DIPEA (113 mg, 0.874 mmol) in sequence. The resulting mixture was stirred at room temperature for further 1 h before it was concentrated in vacuo directly. The intermediate S208 was obtained as a dark yellow oil (797 mg, 100%) and used without further purification. HRMS (ESI): calcd for C₁₃₇H₂₀₀FN₂₀O₃₇⁺ [M+H]⁺ 2736.4362, found 2736.4364.

### Step 13:

To a stirring solution of S208 (797 mg, 0.291 mmol) in CH₂Cl₂ (5 mL) at room temperature was added TFA (10 mL). The resulting mixture was stirred at room temperature for 4 h before it was concentrated in vacuo directly. The product A037 was obtained as a yellow solid (225 mg, 36% over 2 steps) with RP-HPLC and lyophilization. HRMS (ESI): calcd for C₁₁₃H₁₅₂FN₂₀O₃₇⁺ [M+H]⁺ 2400.0606, found 2400.0609.

### Example 369˜379: Synthesis of A035, A039, A041, A043, A045, A047, A049, A051, A053, A055 and A057

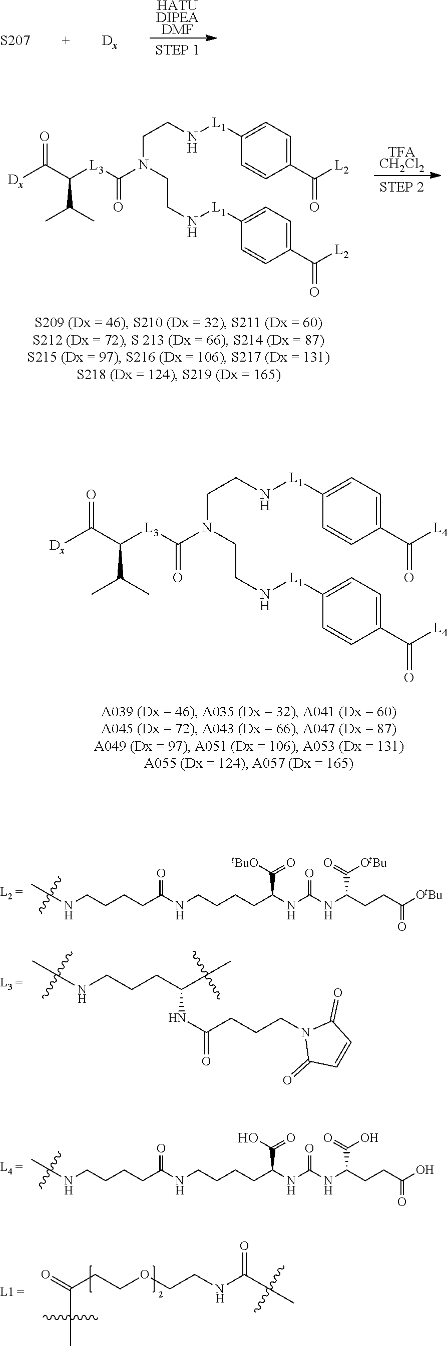

### Step 1˜STEP 2:

Intermediate S₂₀₉˜ S₂₁₉ and product A035, A039, A041, A043, A045, A047, A049, A051, A053, A055 and A057 were prepared according to the procedures in A037 using corresponding component part (S₄₆, S₃₉, S₆₀, S₇₂, S₆₆, S₈₇, S₉₇, S₁₀₆, S₁₃₁, S₁₂₄ and S165).
When Dₓ=S₄₆, product=A039. HRMS (ESI): calcd for C₁₂₆H₁₆₉FN₂₃O₃₈⁺ [M+H]⁺ 2631.1977, found 2631.1980.
When Dₓ=S₃₉, product=A035. HRMS (ESI): calcd for C₁₃₇H₂₀₁FN₁₉O₃₉⁺ [M+H]⁺ 2755.4308, found 2755.4315.
When Dₓ=S₆₀, product=A041. HRMS (ESI): calcd for C₁₁₃H₁₅₃FN₂₁O₃₈⁺ [M+H]⁺ 2431.0664, found 2431.0665.
When Dₓ=S₇₂, product=A045. HRMS (ESI): calcd for C₁₂₇H₁₇₀FN₂₂O₃₉⁺ [M+H]⁺ 2646.1974, found 2646.1979.
When Dₓ=S₆₆, product=A043. HRMS (ESI): calcd for C₁₂₅H₁₆₈FN₂₂O₃₉⁺ [M+H]⁺ 2620.1818, found 2620.1824.
When Dₓ=S₈₇, product=A047. HRMS (ESI): calcd for C₁₁₇H₁₆₂FN₂₀O₄₀⁺ [M+H]⁺ 2506.1236, found 2506.1240.
When Dₓ=S₉₇, product=A049. HRMS (ESI): calcd for C₁₁₈H₁₆₂FN₂₀O₄₁⁺ [M+H]⁺ 2534.1185, found 2534.1186.
When Dₓ=S₁₀₆, product=A051. HRMS (ESI): calcd for C₁₂₀H₁₆₉FN₂₁O₄₁S⁺ [M+H]⁺ 2611.1484, found 2611.1485.
When Dₓ=S₁₃₁, product=A053. HRMS (ESI): calcd for C₁₁₆H₁₅₆FN₂₀O⁺ [M+H]⁺ 2488.0766, found 2488.0766.
When Dₓ=S₁₂₄, product=A055. HRMS (ESI): calcd for C₁₂₅H₁₆₉FN₂₁O₄₁⁺ [M+H]⁺ 2639.1763, found 2639.1764.
When Dₓ=S₁₆₅, product=A057. HRMS (ESI): calcd for C₁₂₆H₁₇₀N21041S⁺ [M+H]⁺ 2665.1578, found 2665.1579.

Example 380˜ 382: Synthesis of A028˜ A030

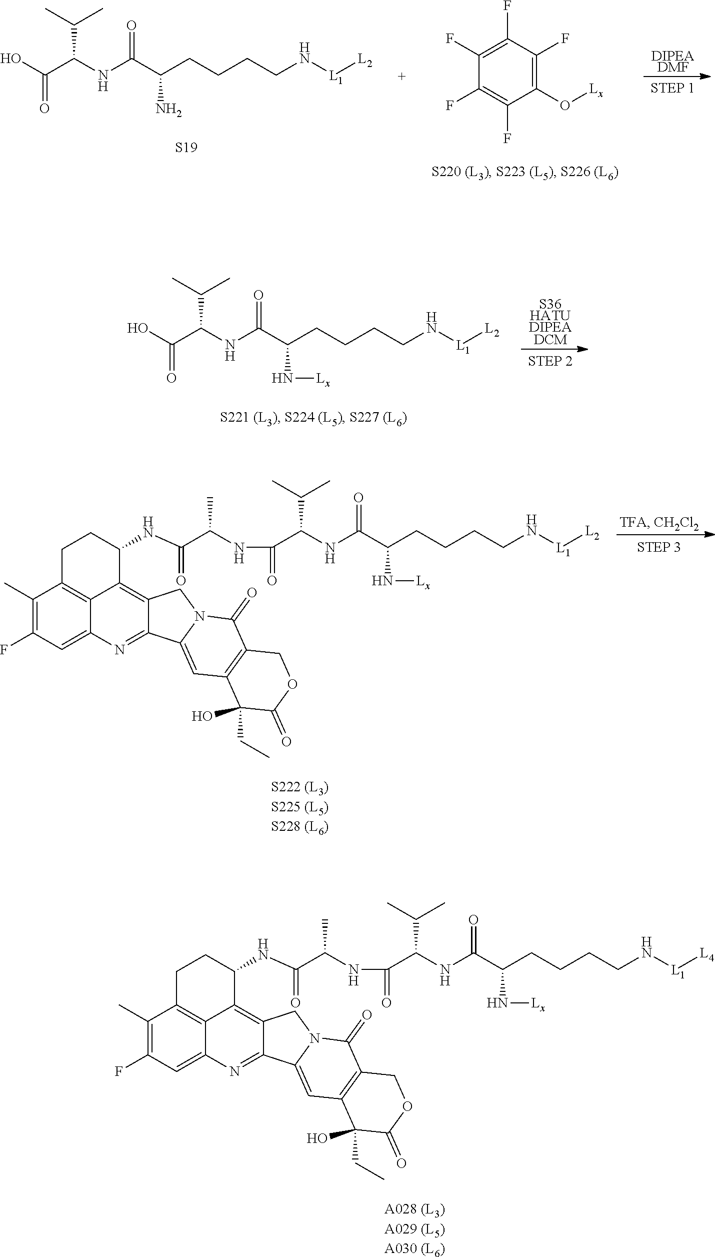

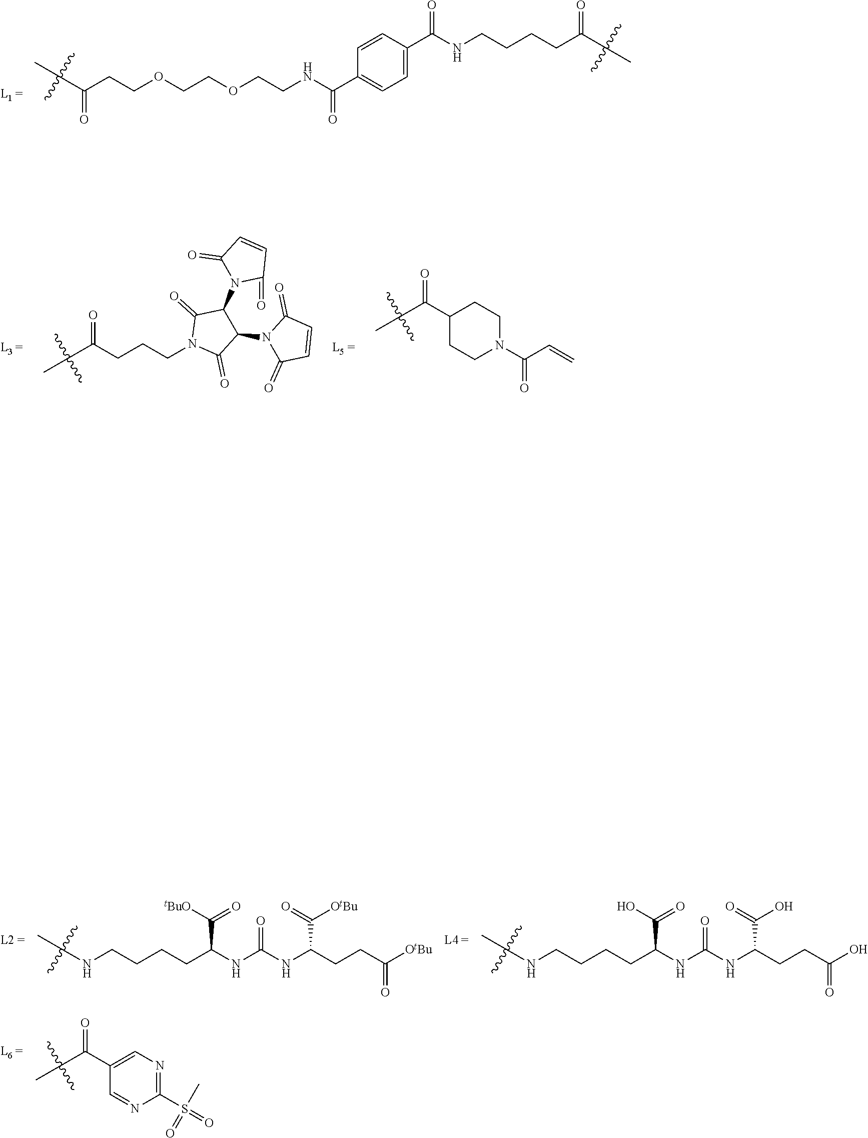

### Step 1:

To a stirring solution of S19 (200 mg, 0.178 mmol) in DMF (2 mL) at room temperature were added S220 (116 mg, 0.214 mmol) and DIPEA (35 mg, 0.214 mmol). The resulting mixture was stirred at room temperature for 3 h before it was concentrated in vacuo directly. The intermediate S221 was obtained as a light yellow solid (232 mg, 88%) after RP-HPLC purification and lyophilization. HRMS (ESI): calcd for C₇₁H₁₀₄N11O₂₃⁺ [M+H]⁺ 1478.7301, found 1478.7302.

To a stirring solution of S221 (232 mg, 0.157 mmol) in DMF (10 mL) at room temperature was added HATU (63 mg, 0.165 mmol). The resulting mixture was stirred at room temperature for 15 min before addition of S36 (103 mg, 0.165 mmol) and DIPEA (61 mg, 0.471 mmol) in sequence. The resulting mixture was stirred at room temperature for further 1 h before it was concentrated in vacuo directly. The intermediate S222 was obtained as a dark yellow oil (304 mg, 100%) and used without further purification. HRMS (ESI): calcd for C₉₆H₁₂₅FN₁₅O₂₇⁺ [M+H]⁺ 1938.8848, found 1938.8852.

### Step 3:

To a stirring solution of S222 (304 mg, 0.157 mmol) in CH₂Cl₂ (2 mL) at room temperature was added TFA (4 mL). The resulting mixture was stirred at room temperature for 4 h before it was concentrated in vacuo directly. The product A028 was obtained as a yellow solid (142 mg, 51% over 2 steps) with RP-HPLC and lyophilization. HRMS (ESI): calcd for C₈₄H₁₀₁FN₁₅O₂₇⁺ [M+H]⁺ 1770.6970, found 1770.6971.

A029 and A030 were prepared according to procedures in A028 using corresponding component part (S₂₂₃ and S226).
A029: HRMS (ESI): calcd for C₇₇H₁₀₁FN₁₃O₂₂⁺ [M+H]⁺ 1578.7163, found 1578.7166.
A030: HRMS (ESI): calcd for C₇₄H₉₄FN₁₄O₂₃S⁺ [M+H]⁺ 1597.6316, found 1597.6315.

### Example 383˜385: Synthesis of A059, A062 and A064

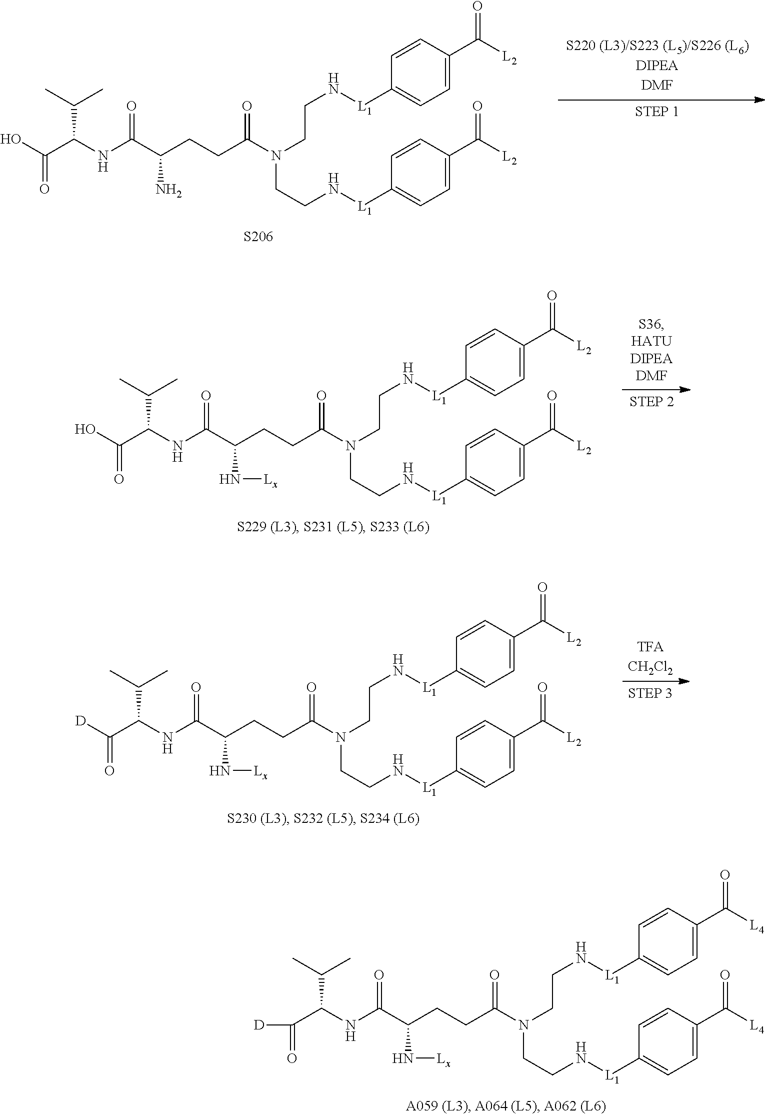

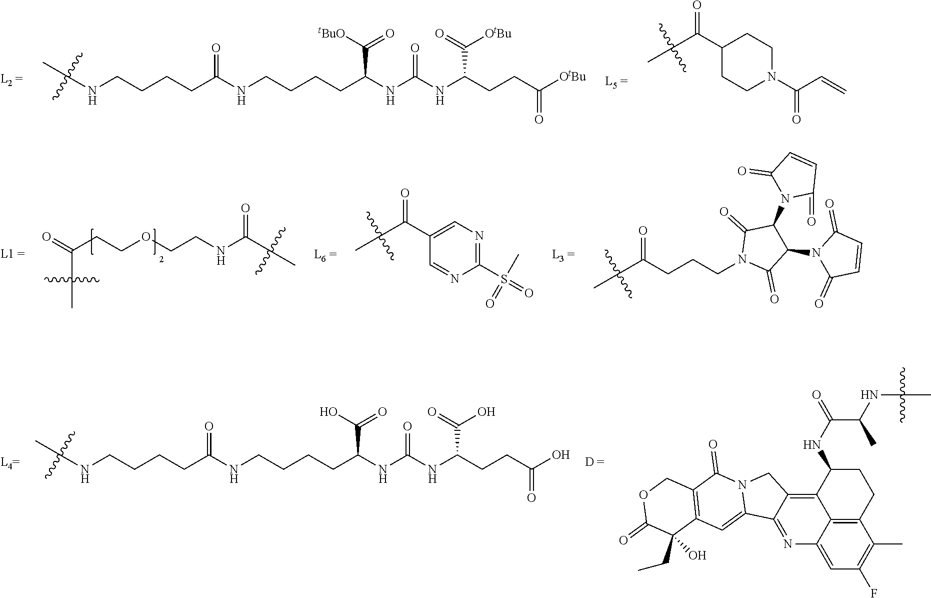

A059, A062 and A064 were prepared according to procedures in A037 using corresponding component part (S₂₂₀, S₂₂₃ and S226).
A059: HRMS (ESI): calcd for C₁₂₁H₁₅₆FN₂₂O₄₁⁺ [M+H]⁺ 2592.0777, found 2592.0778.
A064: HRMS (ESI): calcd for C₁₁₄H₁₅₆FN₂₀O₃₆⁺ [M+H]⁺ 2400.0970, found 2400.0976.
A062: HRMS (ESI): calcd for C₁₁₁H₁₄₉FN₂₁O₃₇S⁺ [M+H]⁺ 2419.0123, found 2419.0123.

### Example 386: Synthesis of A065

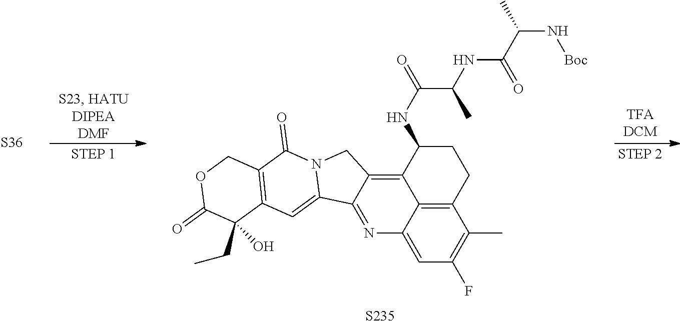

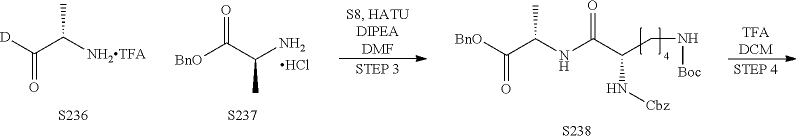

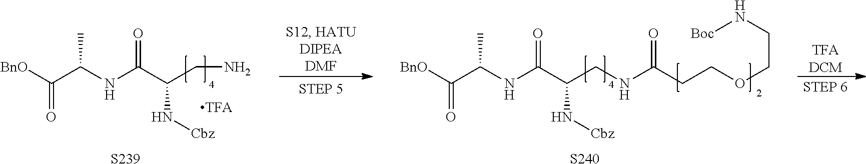

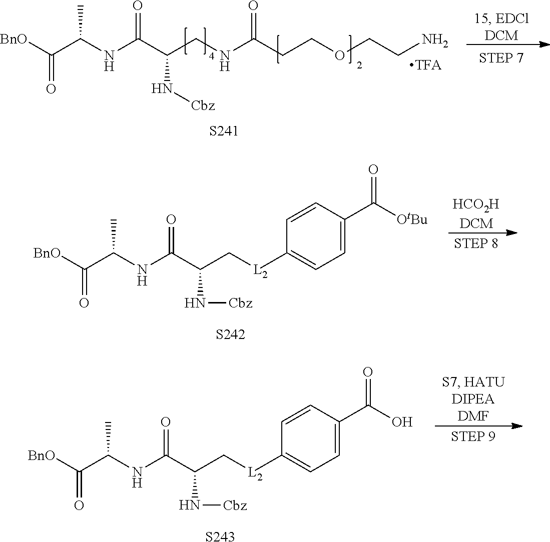

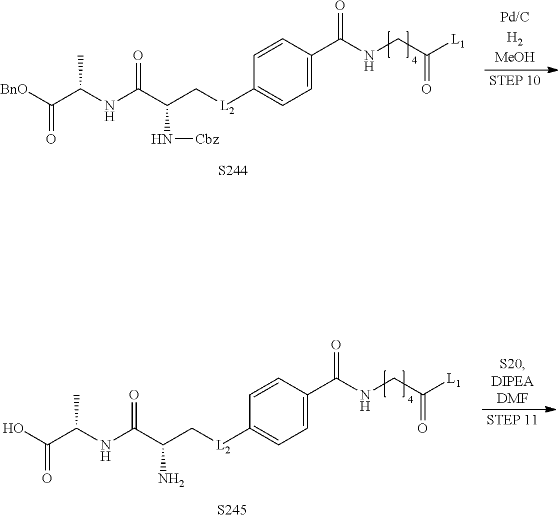

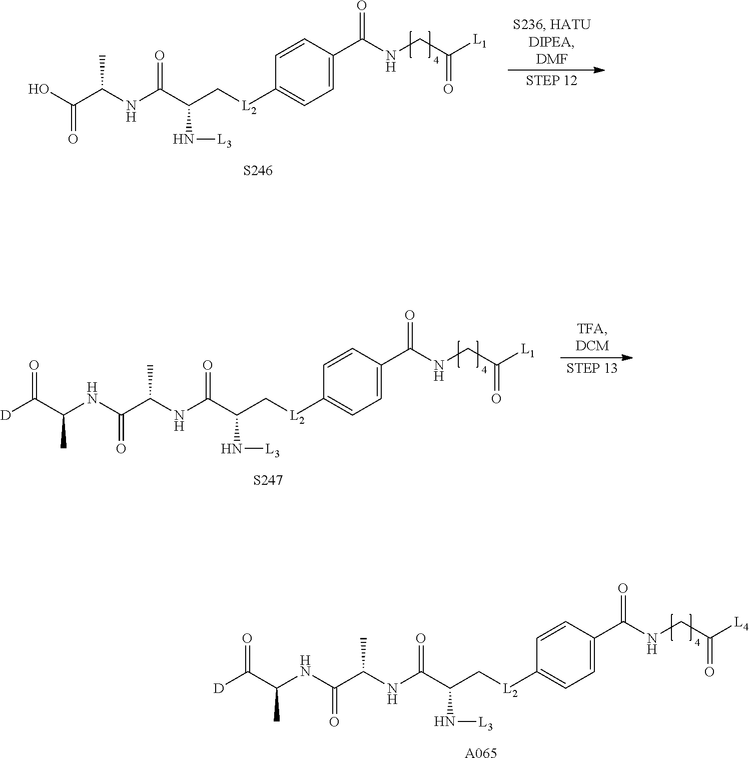

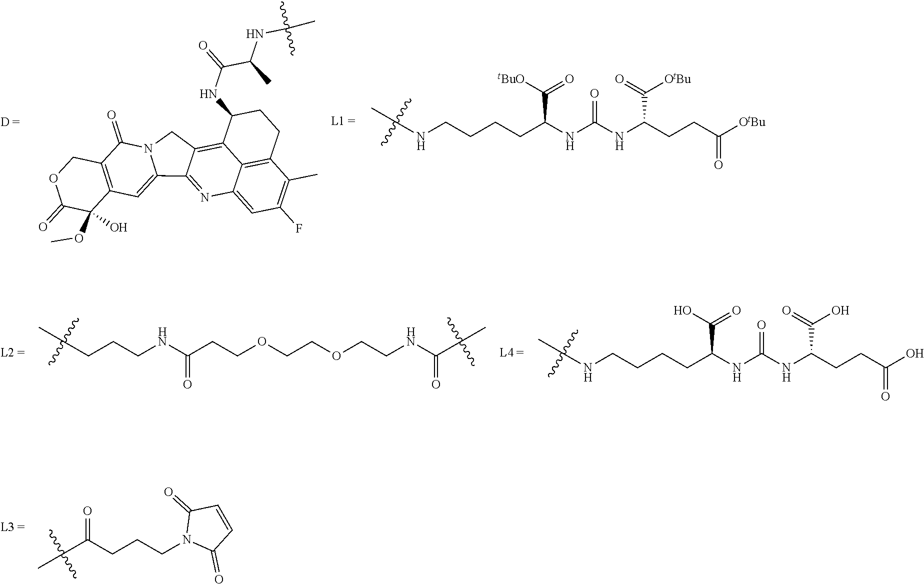 [SMILES: *N[C@@H](C)C(=O)N[C@H]1CCc2c(C)c(F)cc3nc4c(c1c23)Cn1c-4cc2c(c1=O)COC(=O)[C@]2(O)OC ; *NCCCC[C@H](NC(=O)N[C@@H](CCC(=O)OC(C)(C)C)C(=O)OC(C)(C)C)C(=O)OC(C)(C)C ; *CCCNC(=O)CCOCCOCCNC(*)=O ; *NCCCC[C@H](NC(=O)N[C@@H](CCC(=O)O)C(=O)O)C(=O)O ; *C(=O)CCCN1C(=O)C=CC1=O]

### Step 1:

To a stirring solution of S23 (183 mg, 0.967 mmol) in DMF (10 mL) at room temperature were added HATU (368 mg, 0.967 mmol) and DIPEA (313 mg, 2.417 mmol) in sequence. The resulting mixture was stirred at room temperature for 30 min before addition of S34 (500 mg, 0.806 mmol). The resulting mixture was stirred at room temperature for further 1 h before it was quenched with EtOAc (50 mL) and filtered. The filter cake was collected and dried in vacuo. The intermediate S235 was obtained as a yellow solid (513 mg, 93%) and used without further purification. HRMS (ESI): calcd for C₃₅H₄₁FN₅O₈⁺ [M+H]⁺ 678.2934, found 678.2935.

### Step 2:

To a stirring solution of S235 (513 mg, 0.757 mmol) in CH₂Cl₂ (10 mL) at room temperature was added TFA (5 mL). The resulting mixture was stirred at room temperature for 2 h before it was concentrated in vacuo directly. The intermediate S236 was obtained as a yellow solid (480 mg, 91%) after trituration with MTBE (80 mL). HRMS (ESI): calcd for C₃₀H₃₃FN₅O₆⁺ [M+H]⁺ 578.2409, found 578.2410.

### Step 3˜STEP 11:

Intermediate S238˜S246 were prepared according to the procedures in A001 using 237 as corresponding component part.

### Step 12:

To a stirring solution of S246 (200 mg, 0.159 mmol) in DMF (10 mL) at room temperature was added HATU (64 mg, 0.167 mmol). The resulting mixture was stirred at room temperature for 15 min before addition of S236 (116 mg, 0.167 mmol) and DIPEA (52 mg, 0.397 mmol) in sequence. The resulting mixture was stirred at room temperature for further 1 h before it was concentrated in vacuo directly. The intermediate S247 was obtained as a dark yellow oil (288 mg, 100%) and used without further purification. HRMS (ESI): calcd for C₉₁H₁₂₆FN₁₄O₂₄⁺ [M+H]⁺ 1817.9048, found 1817.9050.

### Step 13:

To a stirring solution of S247 (288 mg, 0.158 mmol) in CH₂Cl₂ (2 mL) at room temperature was added TFA (4 mL). The resulting mixture was stirred at room temperature for 4 h before it was concentrated in vacuo directly. The product A065 was obtained as a yellow solid (126 mg, 48% over 2 steps) after RP-HPLC purification and lyophilization. HRMS (ESI): calcd for C₇₉H₁₀₂FN₁₄O₂₄⁺ [M+H]⁺ 1649.7170, found 1649.7174.

### Example 387: Synthesis of A066

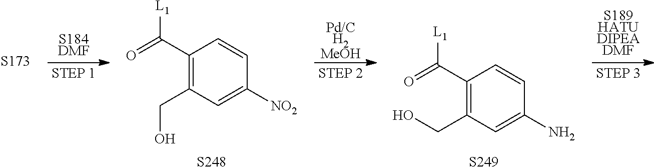

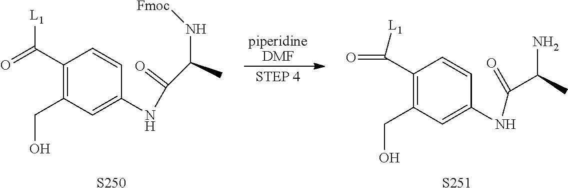

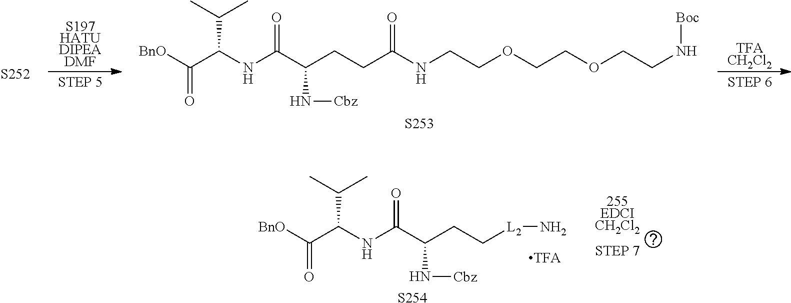

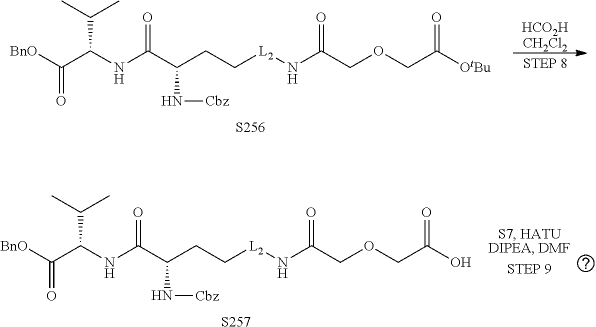

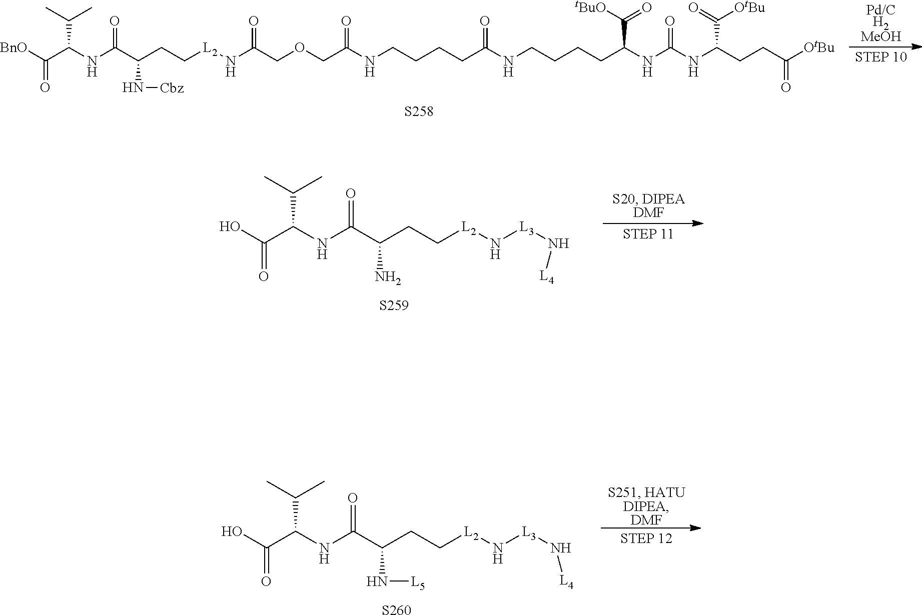

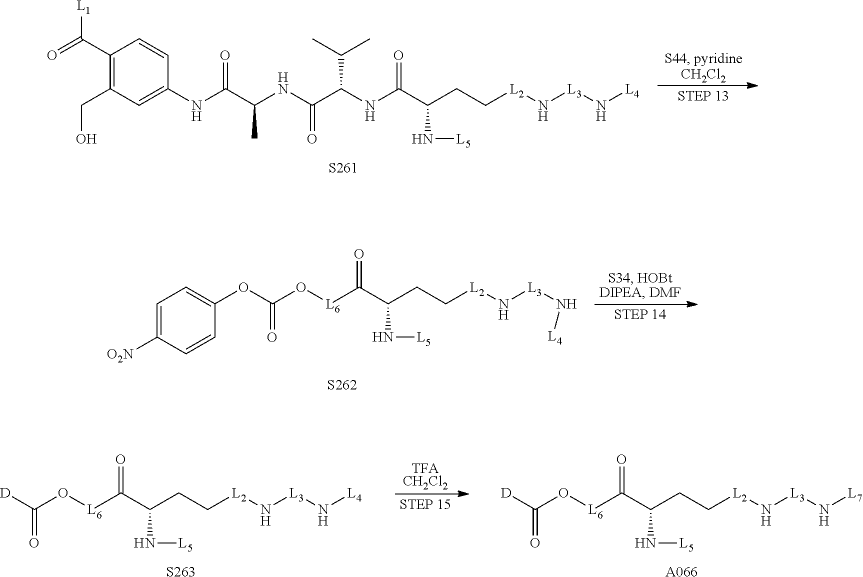

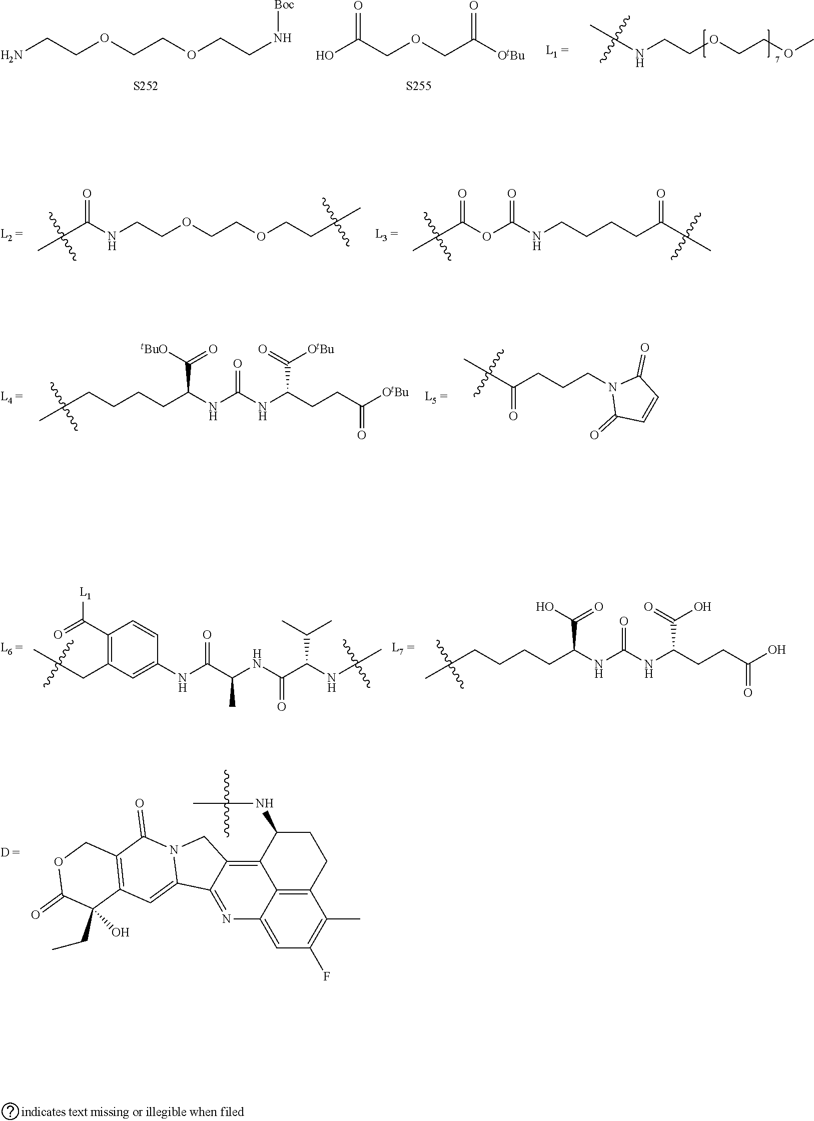

### Step 1:

To a stirring solution of S173 (1.00, 5.583 mmol) in DMF (20 mL) at room temperature was added S184 (2.26 g, 5.893 mmol). The resulting mixture was stirred at room temperature for 24 h before it was concentrated in vacuo directly. The intermediate S248 was obtained as a yellow oil (2.99 g, 95%) after flash column chromatography purification (hexane:EtOAc=2:1). HRMS (ESI): calcd for C₂₅H₄₃N₂O₁₂⁺ [M+H]⁺ 563.2811, found 563.2812.

To a stirring solution of S248 (2.99 g, 5.315 mmol) in MeOH (50 mL) at room temperature was added Pd/C (300 mg, 10% wt/wt). The resulting mixture was stirred at room temperature under H₂ atmosphere for 12 h before it was filtered through a pad of celite. The filtrate was collected and concentrated in vacuo. The intermediate S249 was obtained as a yellow oil (2.83 g, 100%) and used without further purification. HRMS (ESI): calcd for C₂₅H₄₅N₂O₁₀⁺ [M+H]⁺ 533.3069, found 533.3070.

### Step 3:

To a stirring solution of S189 (1.99 g, 6.376 mmol) in DMF (200 mL) at room temperature were added HATU (2.42 g, 6.376 mmol) and DIPEA (1.37 g, 10.627 mmol) in sequence. The resulting mixture was stirred at room temperature for 30 min before addition of S249 (2.83 g, 5.313 mmol). The resulting mixture was stirred at room temperature for further 1 h before it was concentrated in vacuo directly. The residue was dissolved in CH₂Cl₂ (200 mL) again and washed with Na₂CO₃ (5% wt/wt aq., 50 mL) and HCl (1 M aq., 50 mL) in sequence. The organic layer was dried (Na₂SO₄) and concentrated in vacuo. The intermediate S250 was obtained as a yellow oil (4.39 g, 100%) and used without further purification. HRMS (ESI): calcd for C₄₃H₆₀N₃O₁₃⁺ [M+H]⁺ 826.4121, found 826.4122.

### Step 4:

To a stirring solution of S250 (4.39 g, 5.315 mmol) in DMF (100 mL) at room temperature was added piperidine (10 mL). The resulting mixture was stirred at room temperature for 15 min before it was concentrated in vacuo directly. The intermediate S251 was obtained as a yellow oil (2.15 g, 67%) after RP-HPLC purification and lyophilization. HRMS (ESI): calcd for C₂₈H₅₀N₃O₁₁⁺ [M+H]⁺ 604.3440, found 604.3441.

### Step 5:

To a stirring solution of S197 (2.08 g, 4.430 mmol) in DMF (100 mL) at room temperature were added HATU (1.68 g, 4.430 mmol). The resulting mixture was stirred at room temperature for 30 min before addition of S252 (1.00 g, 4.027 mmol) and DIPEA (1.30 g, 10.068 mmol) in sequence. The resulting mixture was stirred at room temperature for further 1 h before it was concentrated in vacuo directly. The residue was dissolved in CH₂Cl₂ (100 mL) again and washed with Na₂CO₃ (5% wt/wt aq., 20 mL) and HCl (1 M aq., 20 mL) in sequence. The organic layer was dried (Na₂SO₄) and concentrated in vacuo. The intermediate S253 was obtained as a yellow oil (2.50 g, 88%) after flash column chromatography purification (silica gel, CH₂Cl₂:MeOH=10:1). HRMS (ESI): calcd for C₃₆H₅₃N₄O₁₀⁺ [M+H]⁺ 701.3756, found 701.3760.

### Step 6˜STEP 11:

Intermediate S₂₅₄˜ S₂₆₀ were prepared according to the procedures in A001 with 255 as corresponding component part.

### Step 12:

To a stirring solution of S260 (2.13 g, 1.739 mmol) in DMF (100 mL) at room temperature were added HATU (662 mg, 1.739 mmol). The resulting mixture was stirred at room temperature for 30 min before addition of S251 (1.00 g, 1.656 mmol) and DIPEA (536 mg, 4.141 mmol) in sequence. The resulting mixture was stirred at room temperature for further 1 h before it was concentrated in vacuo directly. The residue was dissolved in CH₂Cl₂ (100 mL) again and washed with Na₂CO₃ (5% wt/wt aq., 20 mL) and HCl (1 M aq., 20 mL) in sequence. The organic layer was dried (Na₂SO₄) and concentrated in vacuo. The intermediate S253 was obtained as a yellow oil (3.00 g, 100%) after flash column chromatography purification (silica gel, CH₂Cl₂:MeOH=10:1). HRMS (ESI): calcd for C₈₅H₁₄₃N120301 [M+H]⁺ 1812.0028, found 1812.0029.

### Step 13:

To a stirring solution of S253 (3.00 g, 1.656 mmol) in CH₂Cl₂ (50 mL) at 0° C. were added pyridine (262 mg, 3.311 mmol) and S44 (500 mg, 2.483 mmol) in sequence. The resulting mixture was stirred at 0° C. for 2 h before it was quenched with NaHCO₃ (sat. aq., 10 mL). The layers were separated, and the aqueous layer was extracted with CH₂Cl₂ (3× 20 mL). The combined organic layers were dried (Na₂SO₄) and concentrated in vacuo. The intermediate S262 was obtained as a white solid (2.44 g, 74% over 3 steps) after RP-HPLC purification and lyophilization. HRMS (ESI): calcd for C₉₂H₁₄₆N₁₃O₃₄⁺ [M+H]⁺ 1977.0090, found 1977.0092.

### Step 14:

To a stirring solution of S262 (500 mg, 0.253 mmol) in DMF (10 mL) at room temperature were added HOBt (41 mg, 0.303 mmol), S₃₄ (162 mg, 0.303 mmol) and DIPEA (99 mg, 0.759 mmol) in sequence. The resulting mixture was stirred at room temperature for 5 h before it was concentrated in vacuo directly. The intermediate S263 was obtained as a dark yellow oil (575 mg, 100%) and used without further purification. HRMS (ESI): calcd for C₁₁₀H₁₆₃FN₁₅O₃₅⁺ [M+H]⁺ 2273.1415, found 2273.1417.

### Step 15:

To a stirring solution of S263 (575 mg, 0.253 mmol) in CH₂Cl₂ (2 mL) at room temperature was added TFA (4 mL). The resulting mixture was stirred at room temperature for 4 h before it was concentrated in vacuo directly. The product A066 was obtained as a yellow solid (110 mg, 20% over 2 steps) after RP-HPLC purification and lyophilization. HRMS (ESI): calcd for C₉₈H₁₃₉FN₁₅O₃₅⁺ [M+H] 2104.9537, found 2104.9538.

### Example 388: Synthesis of A067

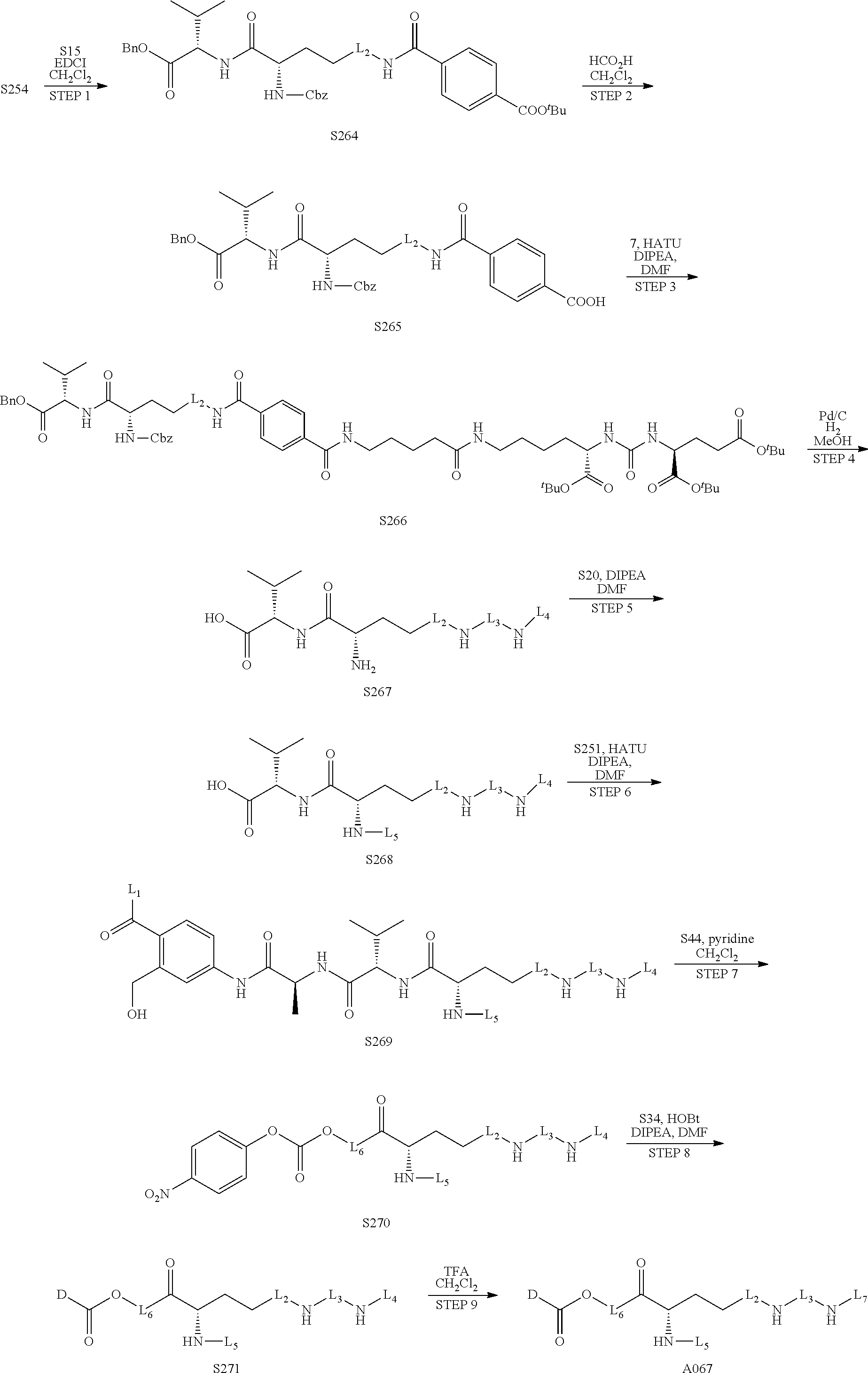

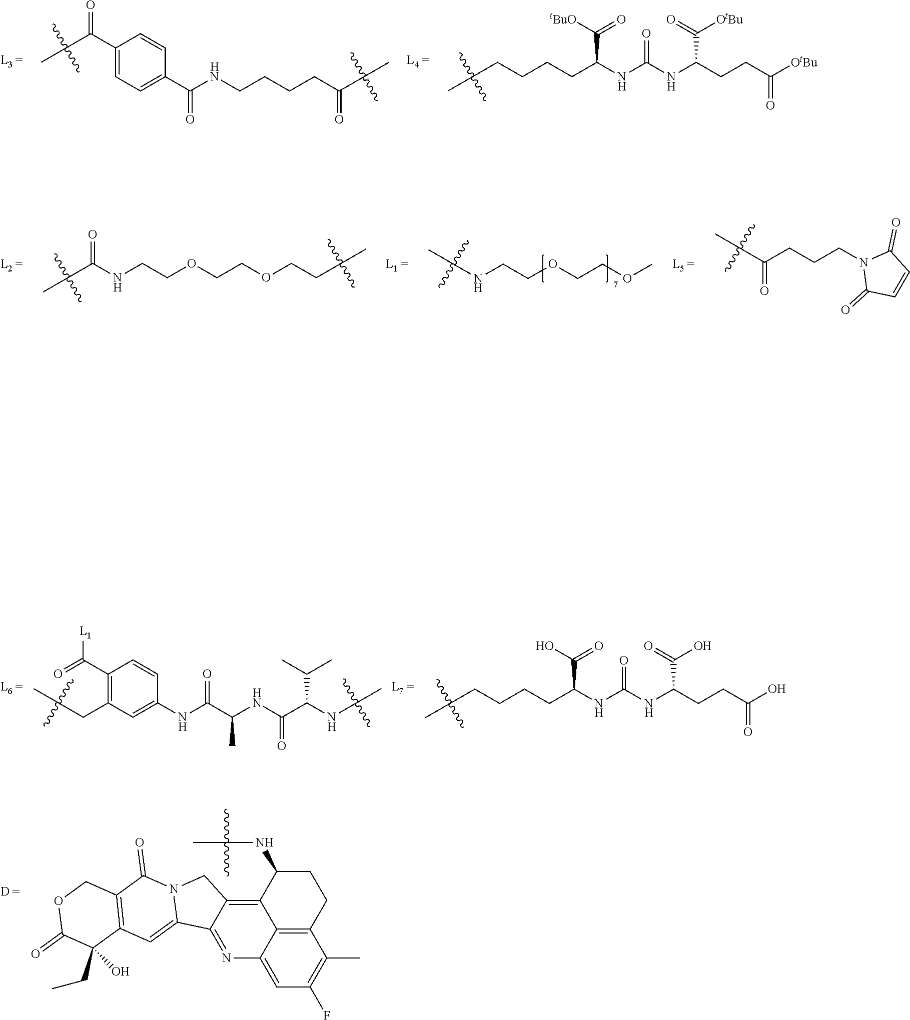

### Step 1˜STEP 9:

Intermediate S₂₆₄˜S₂₇₁ and A067 were prepared according to the procedures in A066 using S₁₅ as corresponding component part.

A067: HRMS (ESI): calcd for C₁₀₂H₁₃₉FN₁₅O₃₄⁺ [M+H]⁺ 2136.9587, found 2136.9592.

### Example 389: Synthesis of A068

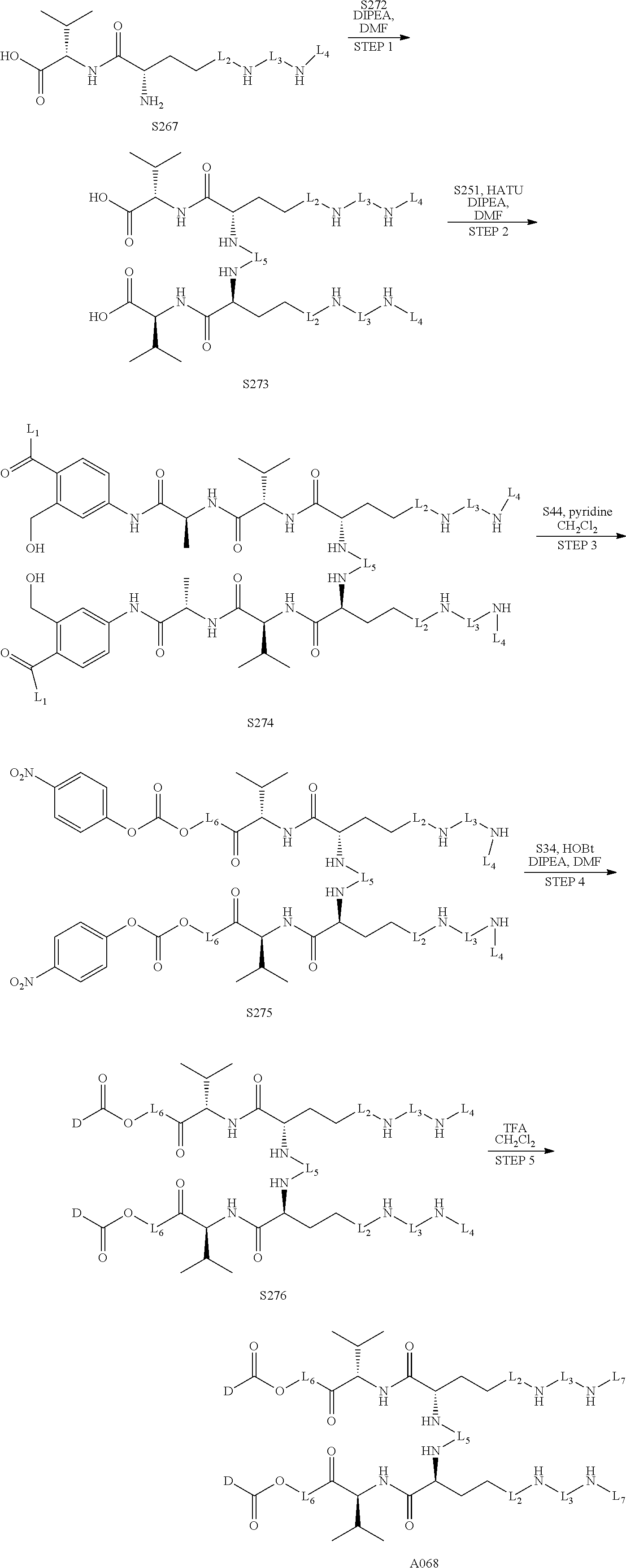

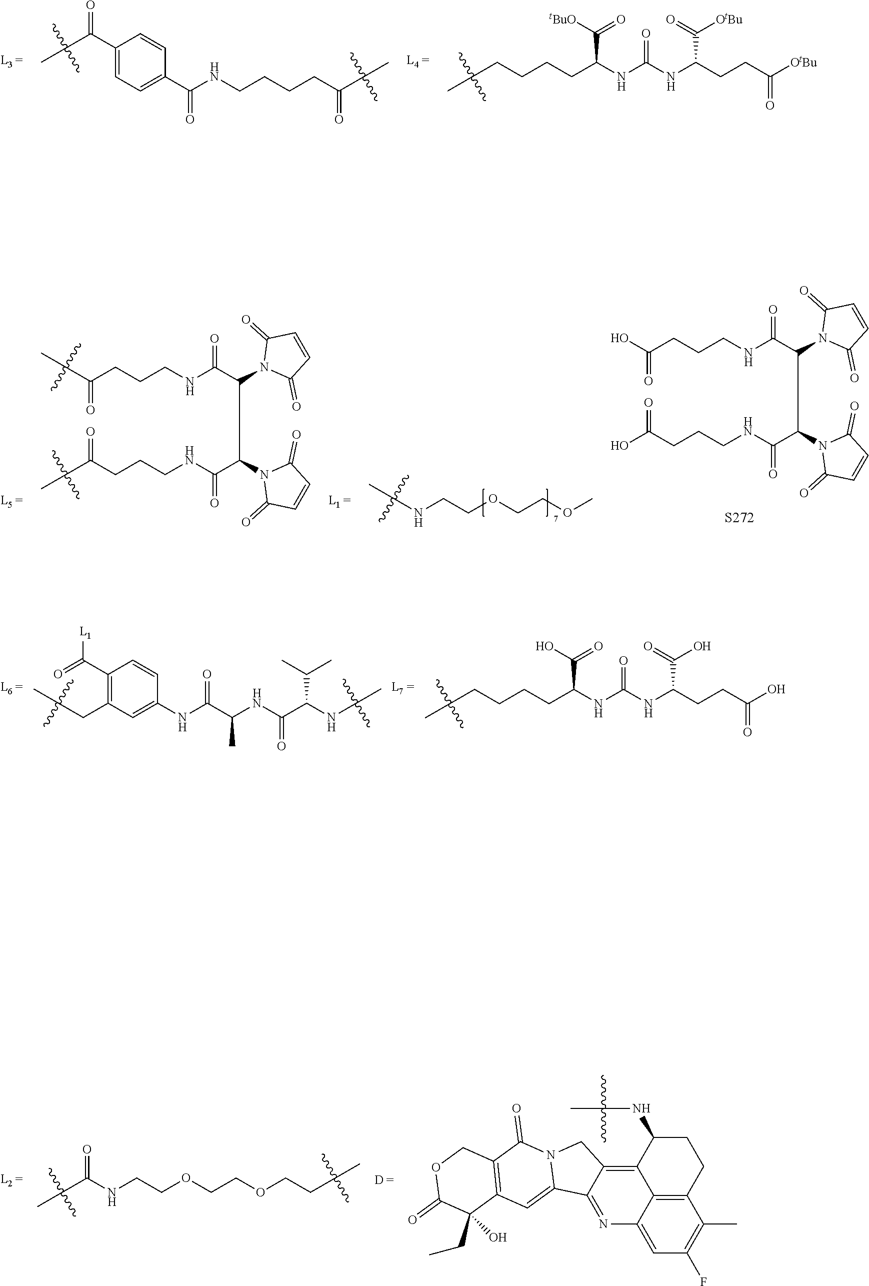

### Step 1˜STEP 5:

Intermediate S₂₇₃˜S₂₇₆ and A068 were prepared according to the procedures in A039 with S₂₇₂ as corresponding component part.

A068: HRMS (ESI): calcd for C₂₀₈H₂₈₁F₂N32O₇₀⁺ [M+H]⁺ 4384.9375, found 4384.9376.

### Example 390˜Example 401: Synthesis of A034, A036, A038, A040, A042, A044, A046, A048, A050, A052, A054 and A056

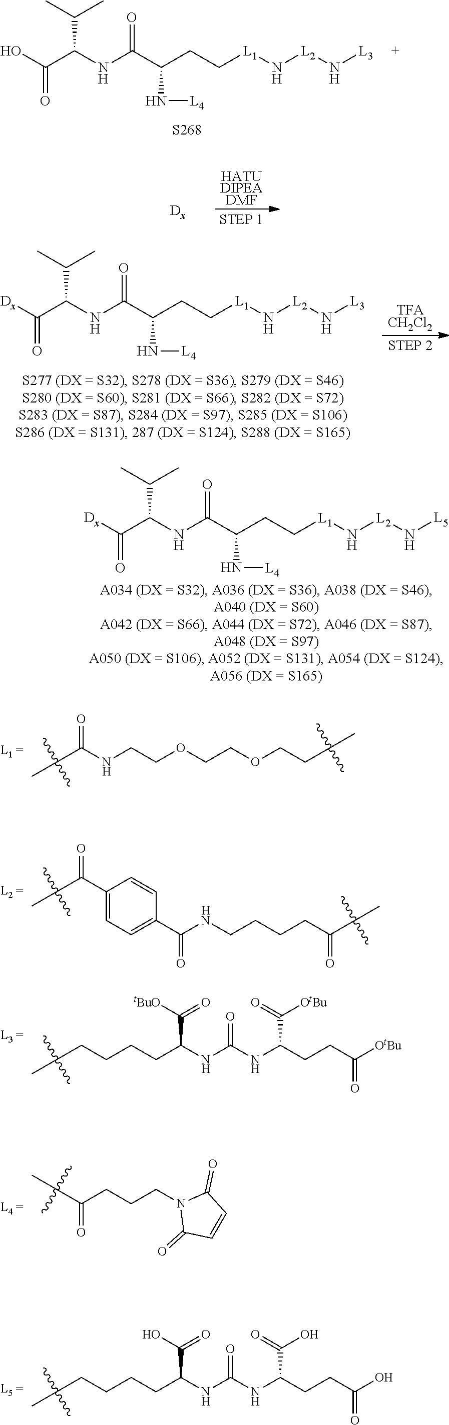

Intermediate S277˜S288 and product A034, A036, A038, A040, A042, A044, A046, A048, A050, A052, A054 and A056 were prepared according to the procedures in A001 using corresponding component part (S₃₂, S₃₆, S₄₆, S₆₀, S₆₆, S₇₂, S₈₇, S₉₇, S₁₀₆, S₁₃₁, S₁₂₄ and S165).
When Dₓ=S₃₂, product=A034. HRMS (ESI): calcd for C₈₃H₁₀₅FN₁₃O₂₅⁺ [M+H]⁺ 1702.7323, found 1702.7324.
When Dₓ=S₃₆, product=A036. HRMS (ESI): calcd for C₇₆H₉₇FN₁₃O₂₃⁺ [M+H]⁺ 1578.6799, found 1578.6801.
When Dₓ=S₄₆, product=A038. HRMS (ESI): calcd for C₈₉H₁₁₄FN₁₆O₂₄⁺ [M+H]⁺ 1809.8170, found 1809.8171.
When Dₓ=S₆₀, product=A040. HRMS (ESI): calcd for C₇₆H₉₈FN₁₄O₂₄⁺ [M+H]⁺ 1609.6857, found 1609.6859.
When Dₓ=S₆₆, product=A042. HRMS (ESI): calcd for C₉₀H₁₁₅FN₁₅O₂₅⁺ [M+H]⁺ 1824.8167, found 1824.8168.
When Dₓ=S₇₂, product=A044. HRMS (ESI): calcd for C₈₈H₁₁₃FN₁₅O₂₅⁺ [M+H]⁺ 1798.8011, found 1798.8012.
When Dₓ=S₈₇, product=A046. HRMS (ESI): calcd for C₈₀H₁₀₇FN₁₃O₂₆⁺ [M+H]⁺ 1684.7429, found 1684.7431.
When Dₓ=S₉₇, product=A048. HRMS (ESI): calcd for C₈₁H₁₀₇FN₁₃O₂₇⁺ [M+H]⁺ 1712.7378, found 1712.7390.
When Dₓ=S₁₀₆, product=A050. HRMS (ESI): calcd for C₈₃H₁₁₄FN₁₄O₂₇S⁺ [M+H]⁺ 1789.7677, found 1789.7678.
When Dₓ=S₁₃₁, product=A052. HRMS (ESI): calcd for C₇₉H₁₀₁FN₁₃O₂₆⁺ [M+H]⁺ 1666.6959, found 1666.6960.
When Dₓ=S₁₂₄, product=A054. HRMS (ESI): calcd for C₈₈H₁₁₄FN₁₄O₂₇⁺ [M+H]⁺ 1817.7956, found 1817.7959.
When Dₓ=S₁₆₅, product=A056. HRMS (ESI): calcd for C₈₉H₁₁₅N₁₄O₂₇S⁺ [M+H]⁺ 1843.7771, found 1843.7775.

### Example 402˜ Example 405: Synthesis of A058, A060, and A061

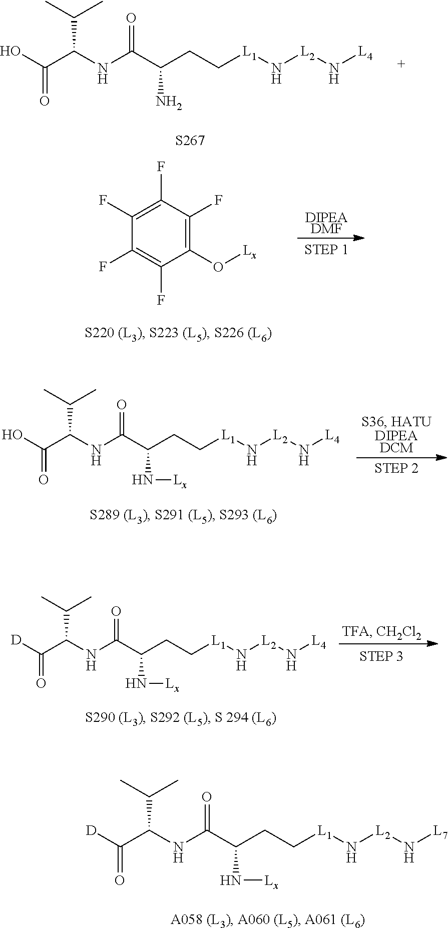

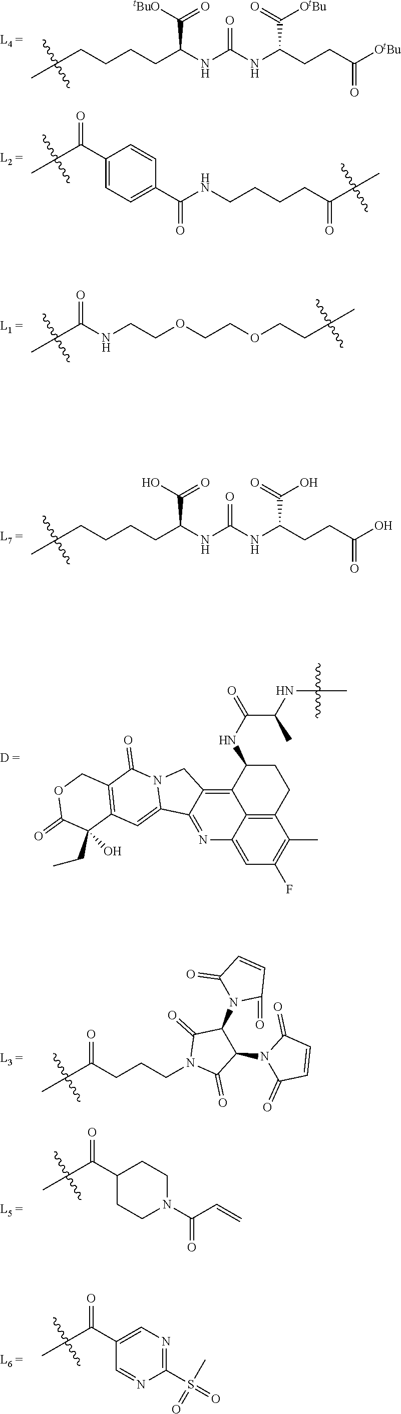

Intermediate S288˜S294 and product A058, A060 and A061 were prepared according to the procedures in A028 with corresponding component part (S₂₂₀, S₂₂₃ and S226).
A058: HRMS (ESI): calcd for C₈₄H₁₀₁FN₁₅O₂₇⁺ [M+H]⁺ 1770.6970, found 1770.6972.
A060: HRMS (ESI): calcd for C₇₇H₁₀₁FN₁₃O₂₂⁺ [M+H]⁺ 1578.7163, found 1578.7166.
A061: HRMS (ESI): calcd for C₇₄H₉₄FN₁₄O₂₃S⁺ [M+H]⁺ 1597.6316, found 1597.6321.

### Example 406: Synthesis of A069

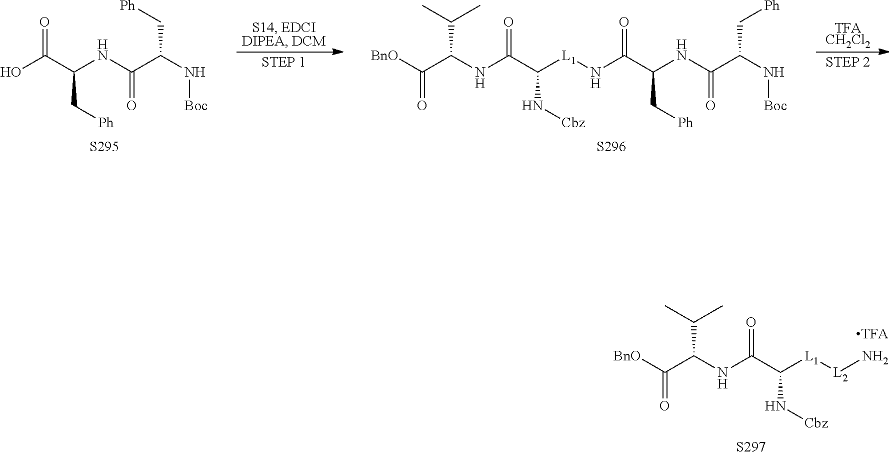

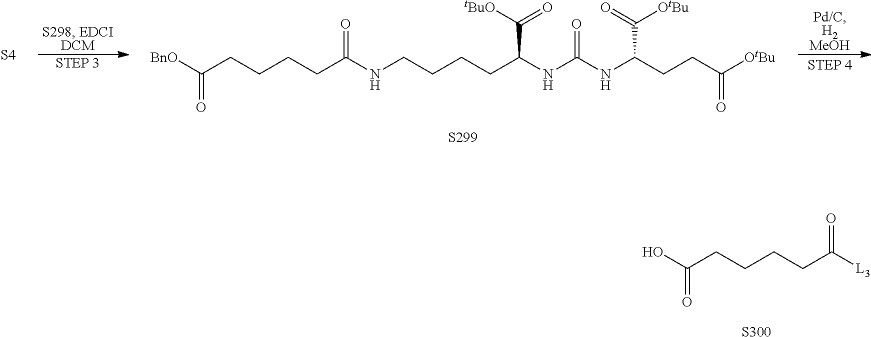

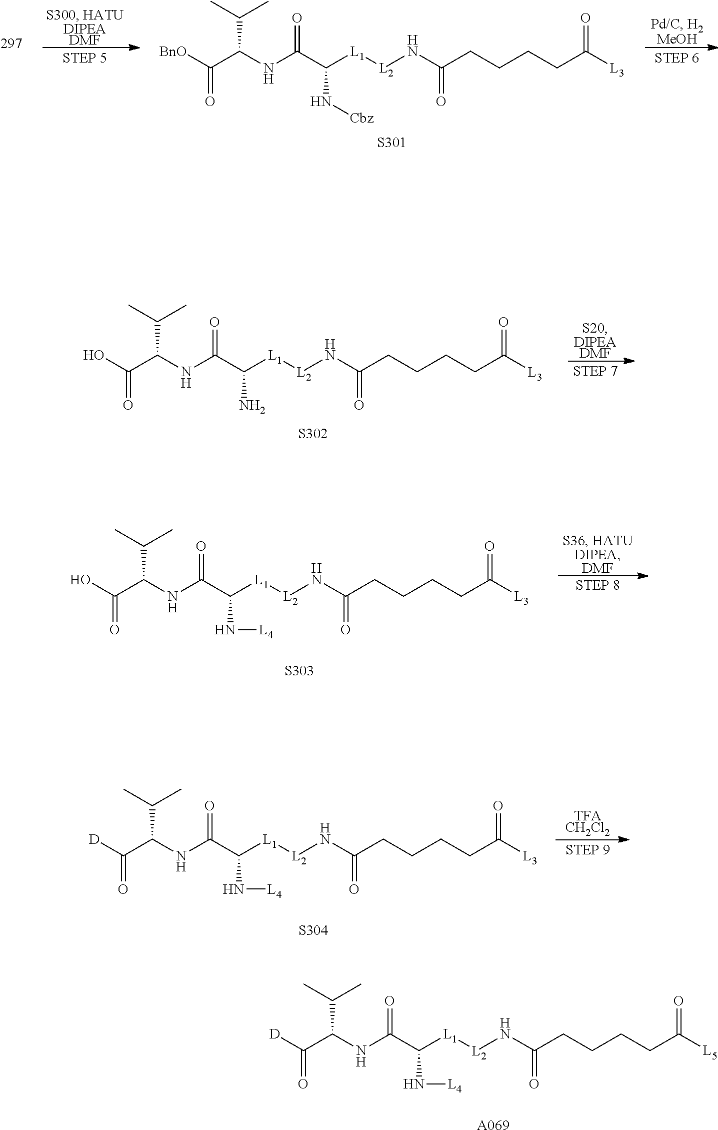

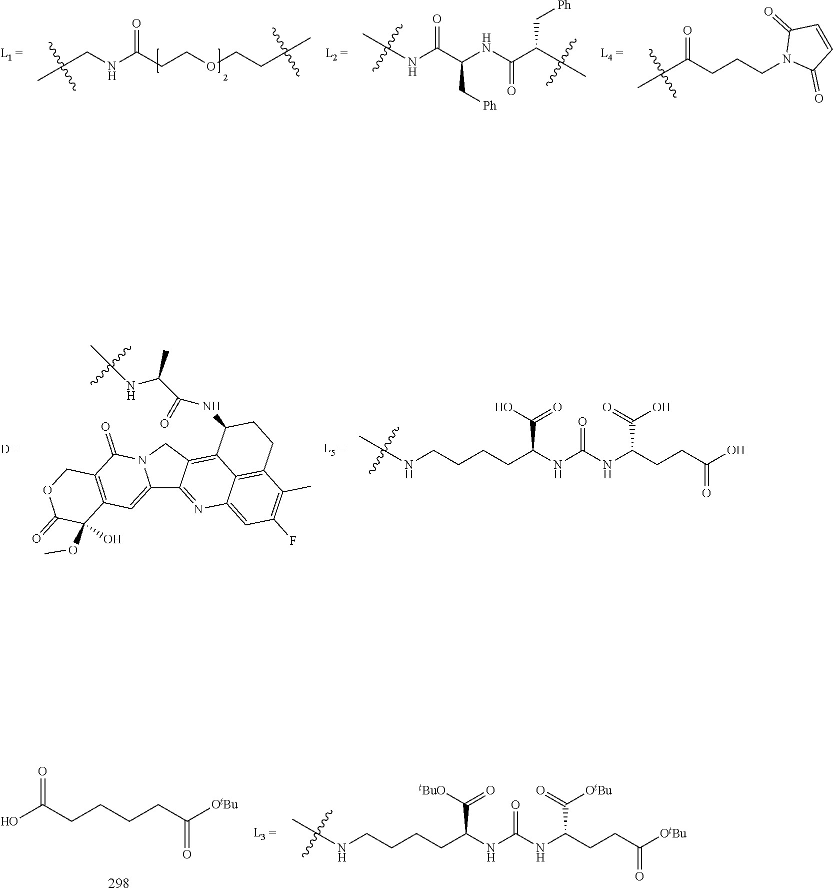

### Step 1:

To a stirring solution of S295 (334 mg, 0.808 mmol) and S14 (500 mg, 0.673 mmol) in CH₂C₁₂ (20 mL) at room temperature were added EDCI (156 mg, 0.808 mmol) and DIPEA (174 mg, 1.346 mmol) in sequence. The resulting mixture was stirred at room temperature for 3 h before it was quenched with Na₂CO₃ (5% wt/wt aq., 5 mL). The layers were separated, and the aqueous layer was extracted with CH₂Cl₂ (3×5 mL). The combined organic layers were dried (Na₂SO₄) and concentrated in vacuo. The intermediate S296 was obtained as a yellow oil (688 mg, 100%) and used without further purification. HRMS (ESI): calcd for C₅₆H₇₅N6O₁₂⁺ [M+H]⁺ 1023.5437, found 1023.5438.

### Step 2:

To a stirring solution of S296 (388 mg, 0.673 mmol) in CH₂Cl₂ (10 mL) at room temperature was added TFA (5 mL). The resulting mixture was stirred at room temperature for 1 h before it was concentrated in vacuo directly. The intermediate S297 was obtained as a yellow solid (400 mg, 57% over 2 steps) after RP-HPLC purification and lyophilization. HRMS (ESI): calcd for C₅₁H₆₇N6O₁₀⁺ [M+H]⁺ 923.4913, found 923.4913.

### Step 3:

To a stirring solution of S298 (249 mg, 1.230 mmol) and S4 (500 mg, 1.025 mmol) in CH₂Cl₂ (20 mL) at room temperature was added EDCI (238 mg, 1.230 mmol). The resulting mixture was stirred at room temperature for 3 h before it was quenched with Na₂CO₃ (5% wt/wt aq., 5 mL). The layers were separated, and the aqueous layer was extracted with CH₂Cl₂ (3× 5 mL). The combined organic layers were dried (Na₂SO₄) and concentrated in vacuo. The intermediate S299 was obtained as a yellow oil (723 mg, 100%) and used without further purification. HRMS (ESI): calcd for C₃₇H₆₀N₃O₁₀⁺ [M+H]⁺ 706.4273, found 706.4276.

### Step 4:

To a stirring solution of S299 (723 mg, 1.025 mmol) in MeOH (10 mL) at room temperature was added Pd/C (73 mg, 10% wt/wt). The resulting mixture was stirred at room temperature under H₂ atmosphere for 2 h before it was filtered through a pad of celite. The filtrate was collected and concentrated in vacuo. The intermediate S300 was obtained as a yellow oil (550 mg, 87%) after RP-HPLC purification and lyophilization. HRMS (ESI): calcd for C₃₀H₅₄N₃O₁₀⁺ [M+H]⁺ 616.3804, found 616.3808.

### Step 5:

To a stirring solution of S300 (200 mg, 0.325 mmol) in DMF (10 mL) at room temperature were added HATU (130 mg, 0.341 mmol) and DIPEA (126 mg, 0.974 mmol) in sequence. The resulting mixture was stirred at room temperature for 20 min before addition of S297 (354 mg, 0.341 mmol). The resulting mixture was stirred at room temperature for further 1 h before it was concentrated in vacuo directly. The residue was dissolved in CH₂Cl₂ (20 mL) again and washed with Na₂CO₃ (5% wt/wt aq., 5 mL) and HCl (1 M aq., 5 mL) in sequence. The organic layer was dried (Na₂SO₄) and concentrated in vacuo. The intermediate S301 was obtained as a dark yellow oil (494 mg, 100%) and used without further purification. HRMS (ESI): calcd for C₈₁H₁₁₈N₉O₁₉⁺ [M+H]⁺ 1520.8538, found 1520.8540.

### Step 6:

To a stirring solution of S301 (494 mg, 0.325 mmol) in MeOH (10 mL) at room temperature was added Pd/C (50 mg, 10% wt/wt). The resulting mixture was stirred at room temperature under H₂ atmosphere for 5 h before it was filtered through a pad of celite. The filtrate was collected and concentrated in vacuo. The intermediate S302 was obtained as a yellow solid (380 mg, 90%) after RP-HPLC purification and lyophilization. HRMS (ESI): calcd for C₆₆H₁₀₆N₉O₁₇⁺ [M+H]⁺ 1296.7701, found 1296.7701.

### Step 7:

To a stirring solution of S302 (300 mg, 0.231 mmol) in DMF (5 mL) at room temperature were added S20 (122 mg, 0.347 mmol) and DIPEA (60 mg, 0.463 mmol) in sequence. The resulting mixture was stirred at room temperature for 2 h before it was concentrated in vacuo. The intermediate S303 was obtained as a white solid (250 mg, 74%) after RP-HPLC purification and lyophilization. HRMS (ESI): calcd for C₇₄H₁₁₃N₁₀O₂₀⁺ [M+H]⁺ 1461.8127, found 1461.8130.

### Step 8:

To a stirring solution of S303 (200 mg, 0.137 mmol) in DMF (10 mL) at room temperature was added HATU (55 mg, 0.144 mmol). The resulting mixture was stirred at room temperature for 15 min before S₃₆ (92 mg, 0.144 mmol) and DIPEA (54 mg, 0.410 mmol) were added in sequence. The resulting mixture was stirred at room temperature for further 1 h before it was concentrated in vacuo directly. The intermediate S304 was obtained as a dark yellow oil (266 mg, 100%) and used without further purification. HRMS (ESI): calcd for C₁₀₁H₁₃₈FN₁₄O₂₄⁺ [M+H]⁺ 1949.9987, found 1949.9990.

### Step 9:

To a stirring solution of S305 (266 mg, 0.137 mmol) in CH₂Cl₂ (2 mL) at room temperature was added TFA (4 mL). The resulting mixture was stirred at room temperature for 4 h before it was concentrated in vacuo directly. The product A069 was obtained as a yellow solid (135 mg, 56% over 2 steps) after RP-HPLC purification and lyophilization. HRMS (ESI): calcd for C₈₉H₁₁₄FN₁₄O₂₄⁺ [M+H]⁺ 1781.8109, found 1781.8110.

### Example 407: Synthesis of A070

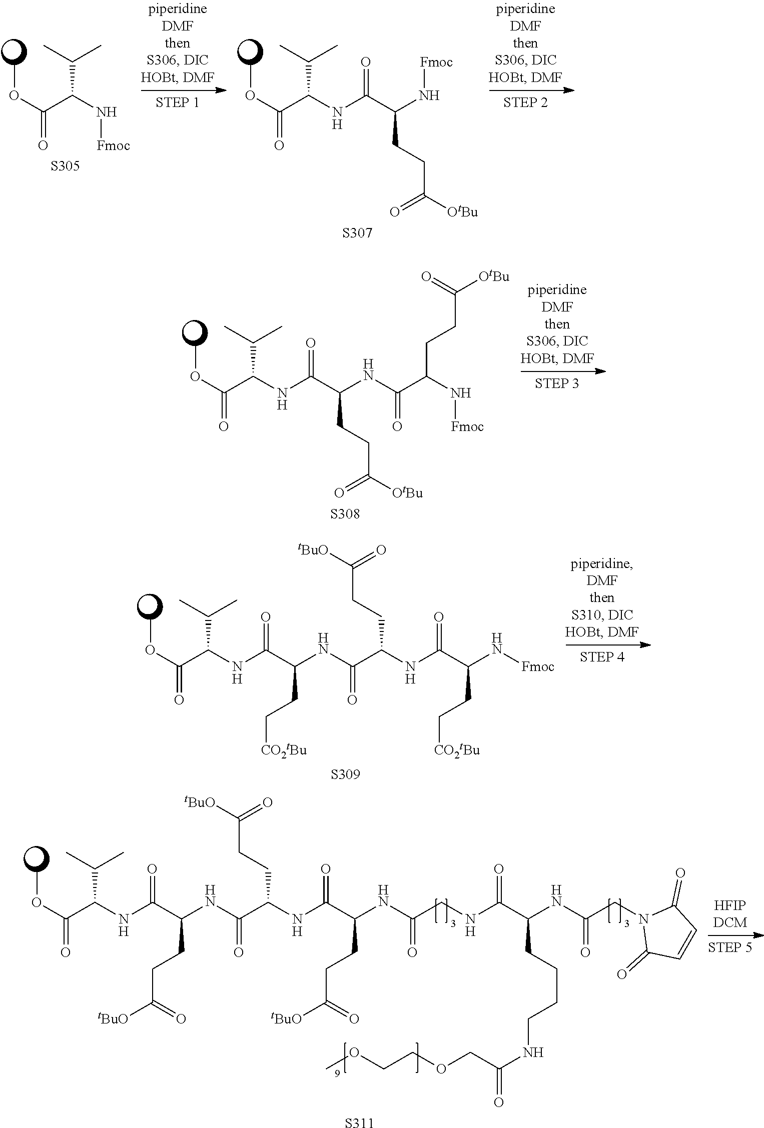

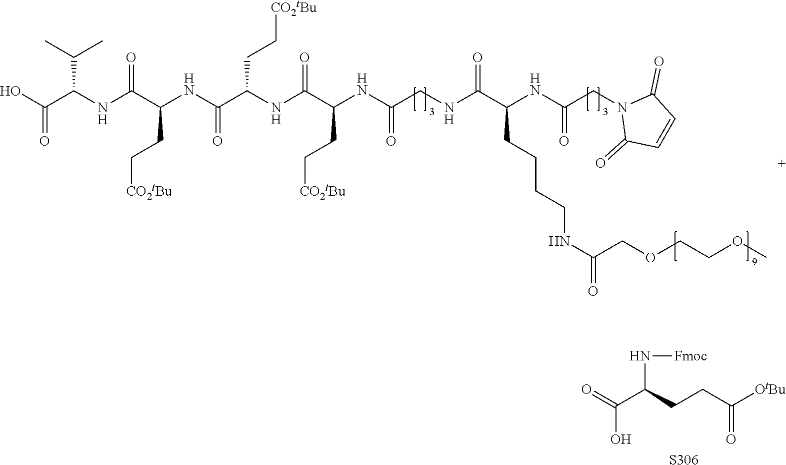

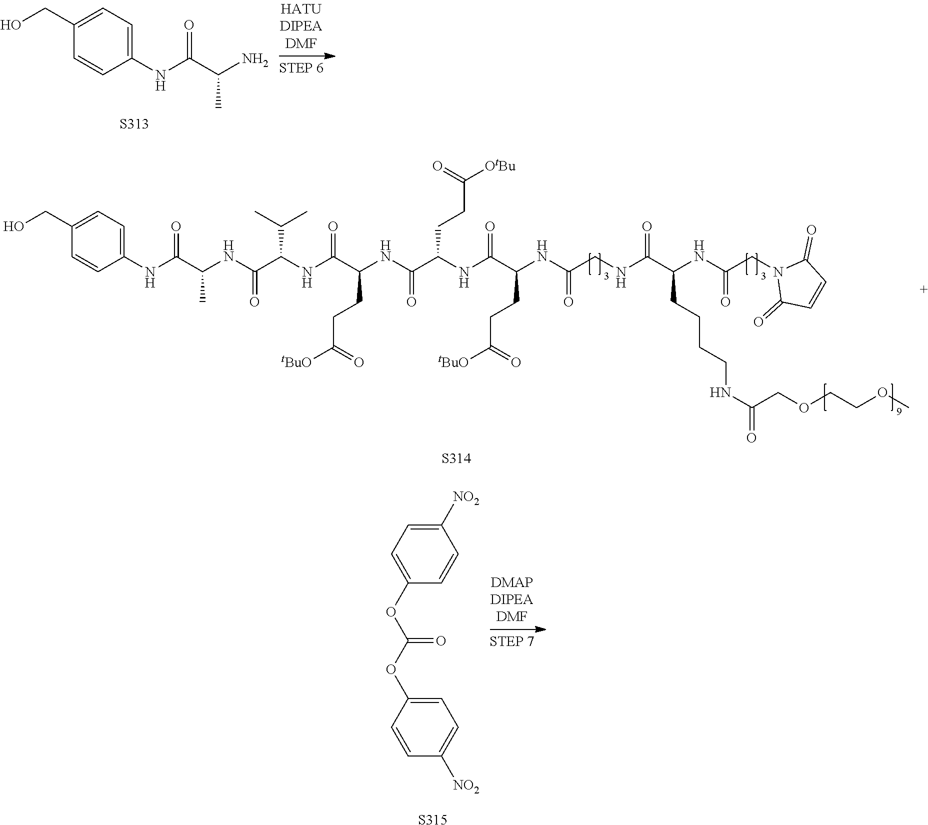

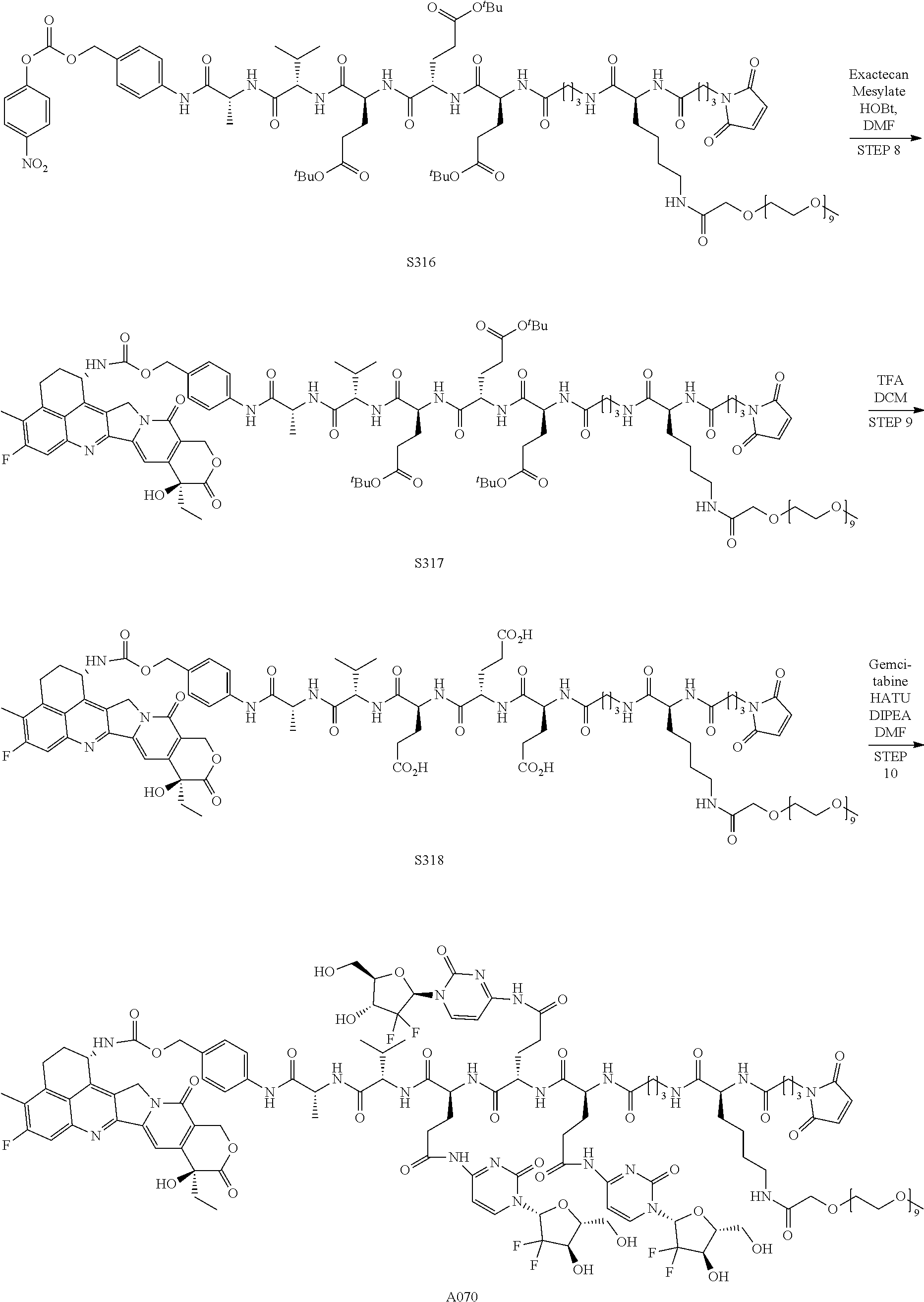

### Step 1˜5:

SPPS synthetic procedures were operated according to general literature guideline. S₃₁₂. HRMS (ESI): calcd for C₇₁H₁₂₃N₈O₂₇⁺ [M+H]⁺ 1519.8492, found 1519.8498. STEP 6:

To a stirring solution of S312 (400 mg, 0.263 mmol) in DMF (10 mL) at room temperature was added HATU (110 mg, 0.289 mmol). The resulting mixture was stirred at room temperature for 30 min before it were added S313 (52 mg, 0.263 mmol) and DIPEA (68 mg, 0.526 mmol) in sequence. The resulting mixture was stirred at room temperature for further 1 h before it was concentrated in vacuo directly. The residue was dissolved in CH₂Cl₂ (20 mL) again and washed with Na₂CO₃ (5% wt/wt aq., 5 mL) and HCl (1 M aq., 5 mL) in sequence. The organic layer was dried (Na₂SO₄) and concentrated in vacuo. The intermediate S314 was prepared as a dark yellow oil (446 mg, 100%) and used without further purification. HRMS (ESI): calcd for C₈₁H₁₃₅N₁₀O₂₈⁺ [M+H]⁺ 1695.9442, found 1695.9444.

### Step 7˜8:

To a stirring solution of S314 (446 mg, 0.263 mmol) in DMF (10 mL) at room temperature were added S315 (96 mg, 0.316 mmol), DMAP (32 mg, 0.263 mmol) and DIPEA (170 mg, 1.315 mmol). The resulting mixture was stirred at room temperature for 2 h before it were added exatecane meslyate (210 mg, 0.394 mmol) and HOBt (54 mg, 0.394 mmol) in sequence. The resulting mixture was stirred at room temperature for further 6 h before it was concentrated in vacuo directly. The intermediate S317 was prepared as a dark yellow oil (142 mg, 25%) and used without further purification. HRMS (ESI): calcd for C₁₀₆H₁₅₅FN₁₃O₃₃⁺ [M+H]⁺ 2157.0829, found 2157.0833.

### Step 9:

To a stirring solution of S317 (142 mg, 0.066 mmol) in CH₂Cl₂ (1 mL) at room temperature was added TFA (1 mL). The resulting mixture was stirred at room temperature for 2 h before it was concentrated in vacuo directly. The residue was dispersed with MTBE (20 mL) and slurrying for 30 min before it was filtered. The filter cake was collected. The intermediate S318 was prepared as a yellow solid (120 mg, 91%) through RP-HPLC and lyophilization. HRMS (ESI): calcd for C₉₄H₁₃₁FN₁₃O₃₃⁺ [M+H]⁺ 1988.8951, found 1988.8952.

### Step 10:

To a stirring solution of 318 (120 mg, 0.060 mmol) in DMF (10 mL) at room temperature was added HATU (83 mg, 0.217 mmol). The resulting mixture was stirred at room temperature for 30 min before it were added gemcitabine (64 mg, 0.241 mmol) and DIPEA (56 mg, 0.434 mmol) in sequence. The resulting mixture was stirred at room temperature for further 1 h before it was concentrated in vacuo directly. The product A070 was prepared as a dark yellow oil (90 mg, 54%) and used without further purification. HRMS (ESI): calcd for C₁₂₁H₁₅₈F₇N₂₂O₄₂⁺ [M+H]⁺ 2724.0787, found 2724.0787.

Example 408. The structures of the conjugates that were prepared by both the traditional conjugation process and the homogeneous conjugation process are illustrated below:
wherein mAb is an antibody, n=1˜20, preferably n=2˜8.
mAbs used for the conjugation have the following information: The Steap1, Trop2, Folate, Her2, and EGFR antibodies were referred from the same of CDRs of vandortuzumab, sacituzumab, farletuzumab, trastuzumab, and nimotuzumab respectively. Her2 bispecfic (trastuzumab/pertuzumab) were referred from the same of CDRs of both trastuzumab and pertuzumab; DLL3, CD54, cMet/EGFR, EGFR/Muc1, Guyc2C (GCC), TF (tissue factor), 5T4, AXL and B7H₃/DLL3 antibodies were generated in house through hybridoma technique and then humanization, and their sequences and production will be filed for patent application later accordingly. For DAR≤6, the homogeneous conjugation process described above was used for preparation of ADCs of this patent. For DAR≥7, the traditional thiol-maleimide conjugation was applied for this application. The structures of the generated ADCs in this patent application are illustrated through the invention.

Experiment 409: Procedure for Recombinant protein/antibody expression, production and purification:

Expression vector construction: DNA sequences encoding the HC and LC of the Steap-1 antibodies were synthesized in General BioL (Anhui, China), and then subcloned into plasmids GS2U and modified pTT5 expression vectors. For transient expression, modified pTT5 vectors containing HC and LC genes were co-transfected into CHO-K1 cells. Cells were cultured for 5 days and supernatant was collected for protein purification using Protein A column or Protein G column. For stable expression, GS2U vectors containing HC and LC genes were co-transfected into CHO-K1 cells. At day 2, cells were diluted to 1×10⁵ cells/mL, and MSX was added to final concentration 25 μM, followed by culture for 10-15 days then subcloned in 96 well plates. Clones with high and stable expression were selected for antibody production at 1 L, and then 5 L, 10 L, 25 L, 200 L or 500 L through various steps of optimization controls of pH, and gases, as well of adjustment of addition of medias, vitamins, metal ions and sugars. After production, the antibody was filtered, purified by Protein A affinity chromatography, anion exchange chromatography and cation exchange chromatography to afford >98% pure, with overall >70% yield of the Steap-1 antibodies.

### Experiment 410: Procedure for Conjugation

Traditional Conjugation: A monoclonal antibody was conjugated to a cytotoxin/linker complex having a terminal of maleimido group. Specifically, purified antibody was incubated with a 2.0-12.0 equivalents of the reducing agent TCEP (Tris(2-carboxyethyl) phosphine) in PBS pH 6.2-7.5, 1 mM EDTA (Ethylenediamine tetraaceticacid) for 1 hours at 37° C. Subsequently, 5.0-12.0 equivalents of the payload of a cytotoxin/linker complex having a terminal of maleimido group from a stock solution in 10% (v/v) DMA or DMSO was added, followed by incubation at room temperature for 1 hour to 3 hours under gentle rotation. The conjugation reaction was optionally quenched by the addition of 4 equivalents (over the payload) of N-acetyl cysteine, and the TCEP was optionally quenched by the addition of 1 or 2 equivalents of 4-azidomethylbenzoic acid or 4-azidobenzoic acid. After incubation, the reducing agent and the excess payload/linker complexes were removed by 2-10 times of dialysis in PBS pH 5.0-7.2, at 4° C. using 20,000 MWCO dialysis cassettes or purified by ion exchange chromatography. For the payload/linker complex containing a disulfide bond, the reduced antibody was isolated through a chromatography (with ion exchange or size exclusion column) or dialysis prior to run conjugation reaction. For Dxd-GGFG conjugation, the conjugates with DAR >7.2 were purified against formulated buffers (normally, 0.02% Tween-20 or Tween-80, 6-7% sugar, 20-50 mM histine, pH=5.5-7.0).

The conjugation process may result in 0.1 to 10% of aggregate formation (e.g. Steap1-GGFG-Dxd ADC). Thus, macromolecular aggregates, conjugation reagents, including payloads quenched by cysteine, and other added regents can be removed using ceramic hydroxyapatite Type II chromatography (CHT) as described in e.g. Thompson et al., J. Control Release, 236:100-116 (2016) or by ion exchange chromatography. The ADCs were optionally formulated in 25 mM Histidine-HCl, or citrate buffer containing 6-7% sucrose, 0.02% Tween-20 or Tween-80, and 0.1% methionine, at pH 5.0-6.5.

### Experiment 411: Preparation of ADC of the Present Invention Via the Homogeneous Conjugation Reaction:

A zinc amino complex (e.g. Zinc 2-methylpropane-1,2-diamine chloride complex) (in 10-60 mM, 1.0-5.0 eq. of an antibody used) and TCEP (in 100 mM, 2.5-7.5 eq. of an antibody used) were added in sequence to a solution containing the antibody (10-50 mg/mL, in 20 mM PBS, pH 5.5-7.5) at 2-8° C. After incubation at 2-8° C. for 12-20 h (overnight), a payload/linker complex (100-200 mM, 2.0-15.0 eq) was introduced and the incubation was continued for further 2-4 h. For the payload/linker contain a disulfide bond, the excess of reductant, such as TCEP, is removed from the reaction mixture through column (affinity, anion or cation exchange) chromatography or UF/DF prior to addition of the payload/linker complex. After the incubation, cystine or 4-(azidomethyl)benzoic acid or 4-azidobenzoic acid (100-200 mM, 4.0-10.0 eq) was added to deplete the excess TCEP; cysteine (100-200 mM, 2.0-10.0 eq) was added to deplete the excess payload/linker complex; EDTA (100-200 mM, 4.0-6.0 eq) was added to trap zinc ion; and DHAA (100-200 mM, 8.0-30.0 eq) was added to oxidize the free thiol groups in the antibody. The reaction mixture was finally purified using a de-salting column (Zeba Spin Desalting Columns, 40K MWCO), or UF/DF, or ion exchange chromatography, and drug/antibody ratios (DAR) were analyzed using HIC-HPLC or HPLC-MS. For the payload/linker complex containing a disulfide bond, the reduced antibody was isolated at 2-8° C. through a chromatography (with ion exchange or size exclusion column) or dialysis prior to running conjugation reaction. In general, average DARs of the invention conjugates by either UV or HIC-HPLC were controlled either 4.0=0.4 or 6.0±0.5. For the two steps of conjugation of two types of payload/linker complexes containing the similar maleimide group or the other thiol reactable groups, the first step reaction can use the homogenous conjugation reaction to conjugate the first functional payload, then the second step is chosen to use Traut's regent to introduce thiols through reaction of a lysine of an antibody and then simultaneously conjugate the second functional payload. It can be performed Traut's regent reaction with the first functional payload, then conducted conjugation of the second functional payload with the homogeneous conjugation reaction.

### Experiment 412: General Formulation of the Conjugates.

In a liquid formulation of 80 mg of each conjugate of the invention in a 10 mL of borosilicate vial containing 240 mg of sucrose and 0.8 mg of Tween-80, 24 mg of sodium citrate in 4 mL of sterile water were adjusted with citric acid to pH 5.5. Then each of the conjugate solution was lyophilized at temperature from −65° C. to 0° C., and to RT at reduced pressure (5˜10 torr) to form a dryness cake. The conjugate cakes were stored at 2˜ 8° C., and then reconstituted with 4 mL of sterile water for further application.

### Experiment 413: PC-3-4H7 and PC-3-4G5, GUCY2C-9C9 Cell Line Development.

For PC-3-4H₇, parent cell line PC-3 from Nanjing Cobioer Biosciences Co., was introduced with pasmid co-expressing both human PSMA and RFP-Neomycin fusion protein and plasmid co-expressing human Steap1 and GFP-Blasticidin fusion protein. The high expression cells were selected by neomycin and Blasticidin, and the selected clone 4H₇ having high expression of both PSMA and Steap1 was verified by FACs and chosen for further in vitro and in vivo studies.

For PC-3-4G5, parent cell line PC-3 was introduced with pasmid expressing RFP-Neomycin fusion protein and plasmid co-expressing human Steap1 and GFP-Blasticidin fusion protein. The high expression cells were selected by neomycin and Blasticidin, and the selected clone 4G5 having high expression of Steap1 was verified by FACs and chosen for further in vitro and in vivo studies.

For GUCY2C-9C₉, parent cell line GUCY2C was introduced with pasmid expressing RFP-Neomycin fusion protein and plasmid co-expressing human GUCY2C and GFP-Blasticidin fusion protein. The high expression cells were selected by neomycin and Blasticidin, and the selected clone 9C₉ having high expression of GUCY2C was verified by FACs and choosen for further in vitro and in vivo studies.

### Experiment 414: Affinity Measurement of the Antibody and ADCs by EELISA.

Antigens were immobilized on the surface of polystyrene microplate wells at a concentration of 1 μg/mL and incubated overnight at 4° C. For blocking, 200 μL of 5% BSA in PBS was added to each well and incubated for 1 hour at 37° C. Subsequently, the wells were washed three times with 300 μL of PBST. The antibody or the ADC was then diluted to the starting concentration, followed by serial dilutions, and added to the microplate wells. The immobilized antigens were incubated with antigen-specific primary antibodies that affinity-bound to the antigens. Afterward. a HRP-conjugated secondary antibody was added and incubated for 1 hour. The microplate wells were then washed three times with 300 μL of PBST. Following TMB color development, the absorbances were measured using a microplate reader.

### Experiment 415: Characterization of the Antibody and the ADC Conjugate.

To determine monomeric content, aggregates, and fragments of ADCs, analytical size-exclusion chromatography (SEC-HPLC) was performed using 100 μg (100 μL volume) of antibodies or ADCs, which were loaded into a TSKgel® G3000WXL column (Tosoh Bioscience, Tokyo, Japan). The mobile phase was composed of 0.1 M sodium sulfate, 0.1 M sodium phosphate, and 10% isopropanol, pH 6.0˜7.0. The flow rate was 1 mL/min, and each analysis was carried out for 10-45 minutes at room temperature.

The monomer purity of the invention (Steap1-conjugates, C01, C02, C03, C04, C05, C06, C07, C08, C09 and C010) was >98% by SEC.

### Experiment 416: Reduced Molecular Weight and DAR Analysis for the Deglycosylated ADCs by LC-MS.

Sample preparation: Reduction of an ADC with 5 mM dithiothreitol at 37° C. for about 2 h, followed by a deglycosylation step with PNGase F at 37° C. overnight generated six or more fragments. HC and LC existed as naked or conjugated forms carrying some payloads. The masses of each ADC fragments and the average DARs of the ADC can be detected. The following equation was used for average DAR calculation for conventional conjugated ADC.

$$Average DAR = L 1 / ( L 0 + L 1 ) \times 2 + H 1 / ( H 0 + H 1 + H 2 + H 3 ) \times 2 + H 2 / ( H 0 + H 1 + H 2 + H 3 ) \times 2 + H 3 / ( H 0 + H 1 + H 2 + H 3 ) \times 2 .$$

Method conditions: UPLC system: Waters ACQUITY UPLC H-Class System; Detector: ACQUITY UPLC TUV; Absorption wavelength: 280 nm; Trap column: ACQUITY UPLC C4 1.7 μm 2.1×50 mm Column; Mobile phase A: 0.1% formic acid (FA) in water, phase B: 0.1% formic acid (FA) in ACN; Performed the chromatographic separation at a flow rate of 0.4 ml/min using a linear gradient of mobile phase B (ACN with 0.1% FA) from 5% to 25% for 2 min, followed by 25% to 45% for 8 min, then 45% to 85% for 2 min.

MS conditions: MS system: Waters Xevo-G2XS Q-TOF; Ionization mode: ESI positive; Mass range: m/z 500-4000 Da. Informatics: the data analysis using UNIFI V1.8.2.169 Software (Waters).

Experiment 417: DAR analysis by HIC-HPLC:

DAR was analyzed by using HIC-HPLC, and the HPLC parameters are as follow Table 1:

TABLE 1
The condition for DAR analysis by HIC-HPLC.

| HPLC | Agilent 1260 | | | | | |
|---|---|---|---|---|---|---|
| Column | Thermo HIC butyl 4.6 × 100 mm | | | | | |
| Phase A | 0.5M (NH₄)₂SO₄ + 100 mM NaH₂PO₄, | | | | | |
| Phase B | 100 mM NaH₂PO₄, | | | | | |
| Sample | Dilute with buffer A to about 2 mg/mL, injection volume | | | | | |
| | 10 μL | | | | | |
| Rate | 0.8 mL/min | | | | | |
| Wavelength | 280 nm | | | | | |
| Column Temp. | 30° C. | | | | | |
| Gradient | Time (min) | 0 | 35 | 40 | 41 | 45 |
| | Phase A (%) | 80 | 0 | 0 | 80 | 80 |
| | Phase B (%) | 20 | 100 | 100 | 20 | 20 |

### Experiment 418: Drug Conjugation Site Analysis by MS

### Sample Preparation

A 500 μg sample was dissolved in Urea (to a final concentration of approximately 5.9 mol/L), followed by the addition of an appropriate amount of DTT (final concentration 9.8 mmol/L). The reaction system was placed in a water bath at 56° C. for 40 minutes to denature and reduce the sample. After denaturation and reduction, the sample was removed from the water bath and allowed to cool to room temperature. An appropriate amount of IAM (to a final concentration of approximately 29 mmol/L) was added, and the sample was reacted at room temperature in the dark for 40 minutes to alkylate and block free thiol groups. Following the alkylation reaction, the sample was diluted with six volumes of 50 mmol/L Tris buffer (pH 7.0). The sample was then mixed with Trypsin enzyme at a 50:1 (w/w) ratio and incubated at 37° C. for 4 hours to perform the enzymatic digestion. The digestion was quenched by adding formic acid to a final concentration of approximately 0.5% (v/v) for subsequent analysis.

### LC/MS^{E} Condition

Data acquisition was performed using a Waters ACQUITY UPLC ultra-performance liquid chromatography system interfaced with a Waters Xevo-G2XS Q-TOF mass spectrometer. A Waters Acquity UPLC Protein BEH C18, 1.7 μm, 2.1×100 mm column was used. A volume of 5 μL of the digested ADC solution was injected onto the column with a 0.2 mL/min flow rate and set the column temperature at 60° C. The mobile phase system was composed of the following: mobile phase A was HPLC-grade water with 0.1% formic acid, mobile phase B was HPLC-grade acetonitrile with 0.1% formic acid. The gradient program consisted of a 95 min linear gradient from 1% to 40% B, followed by an increase to 80% B in 10 min, then a 5 min hold at 80% B, next a decrease to 1% B in 1 min, finally re-equilibrating at 1% B for 9 min.

The Waters Xevo-G2XS Q-TOF mass spectrometer is operated in sensitive mode with a capillary voltage of 3.0 kV. The sample cone was set to 40 V. Source and desolvation temperature were set at 100 and 300° C., respectively. Desolvation and cone gas flows were set at 500 and 50 L/h, respectively. Mass range was from 100 m/z to 2500 m/z. Low energy CE and High energy CE were set at 6 and 15-45 V, respectively.

The mass raw data analysis was conducted using the UNIFI software.

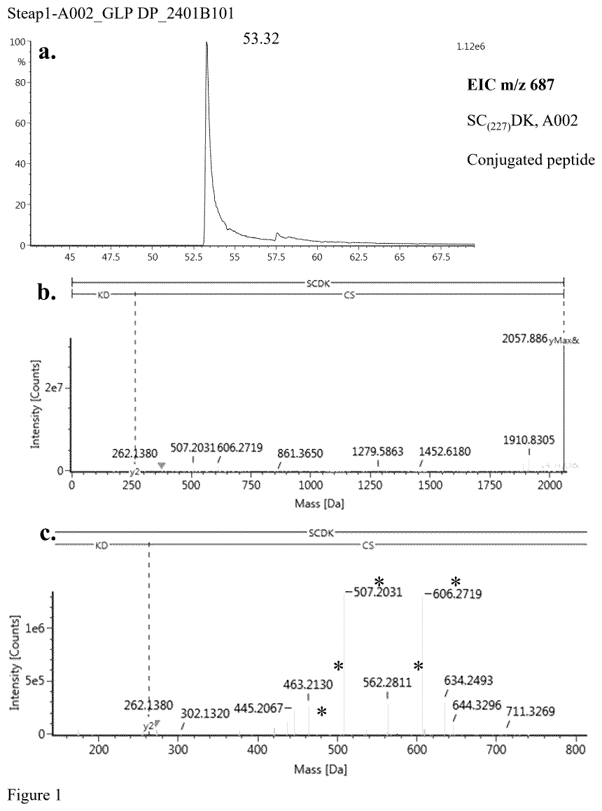

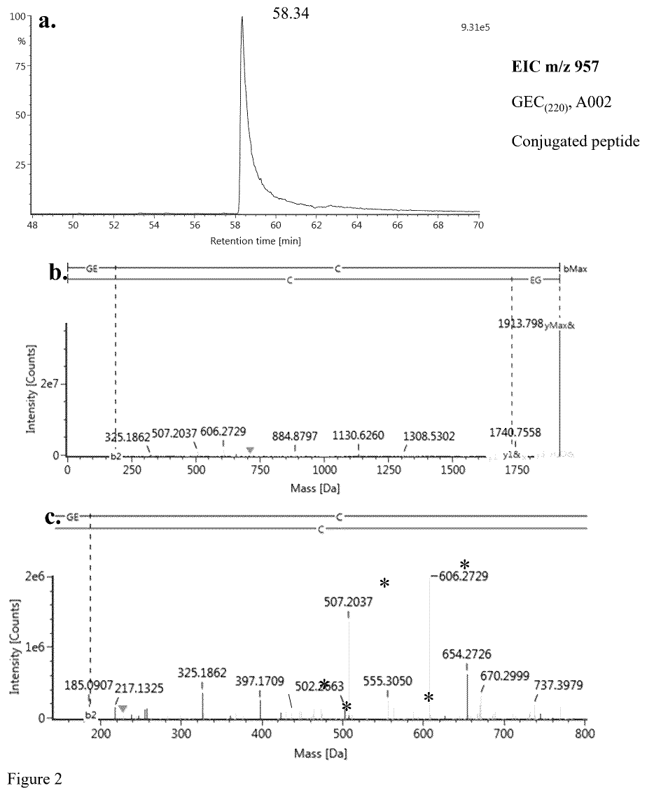

As data indicated in FIGS. 1 and 2, and in Table 2, by using liquid chromatography-mass spectrometry (LC-MSF), the drug conjugation sites in vandortuzumab-ADCs were identified. The primary drug conjugation sites of the Steap1-ADC were almost exclusively located at the cysteine residues SC (227) DK of the heavy and GEC (220) of the light chain, when DAR was control at ˜4.0 by homogeneous conjugation.

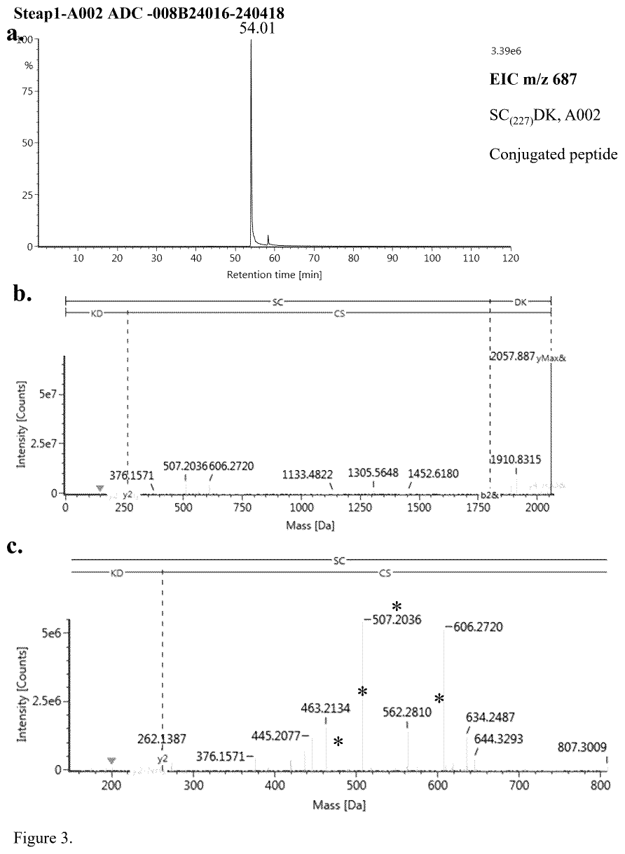

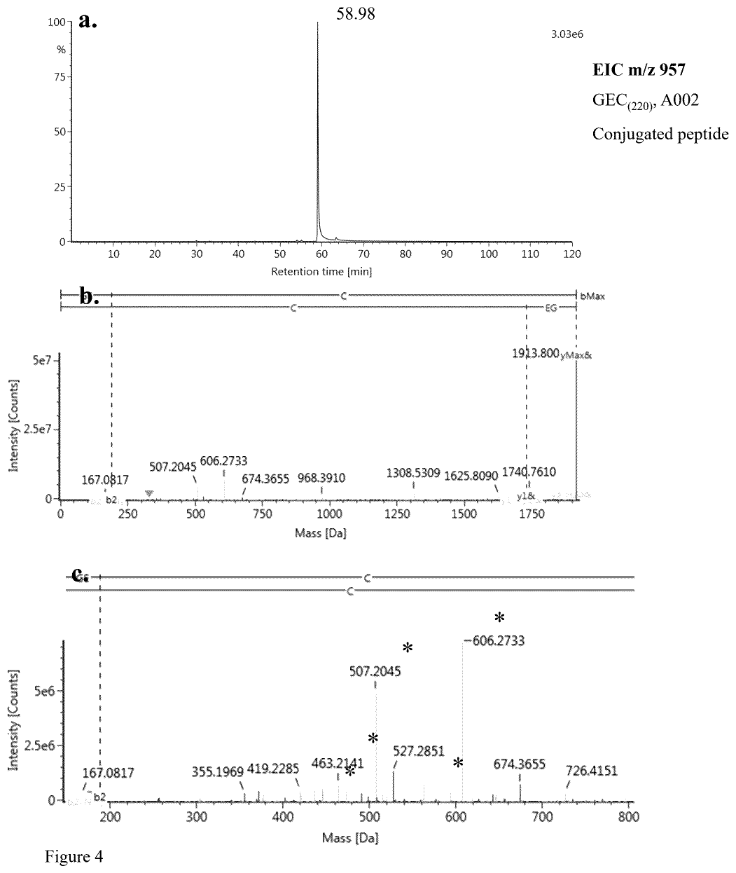

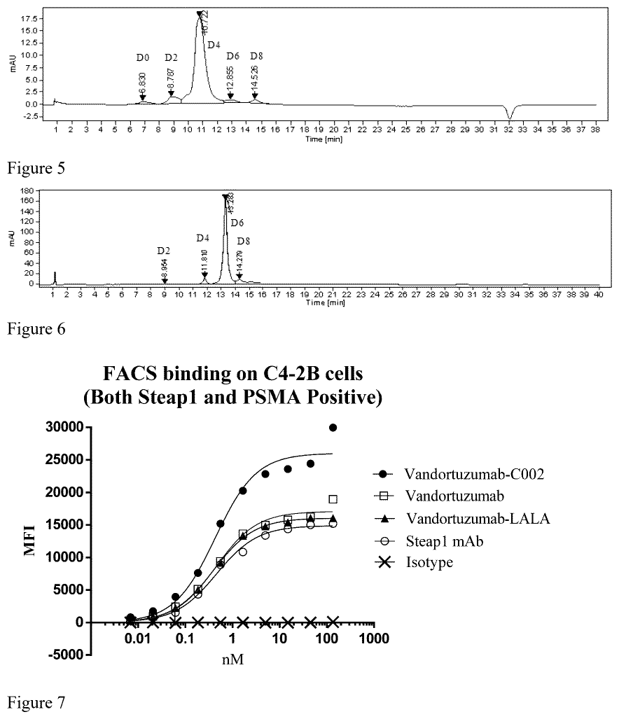

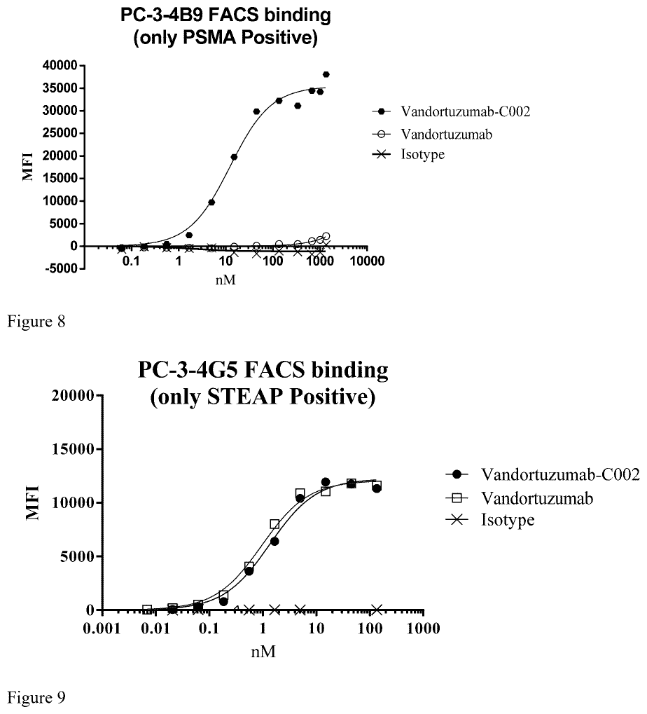

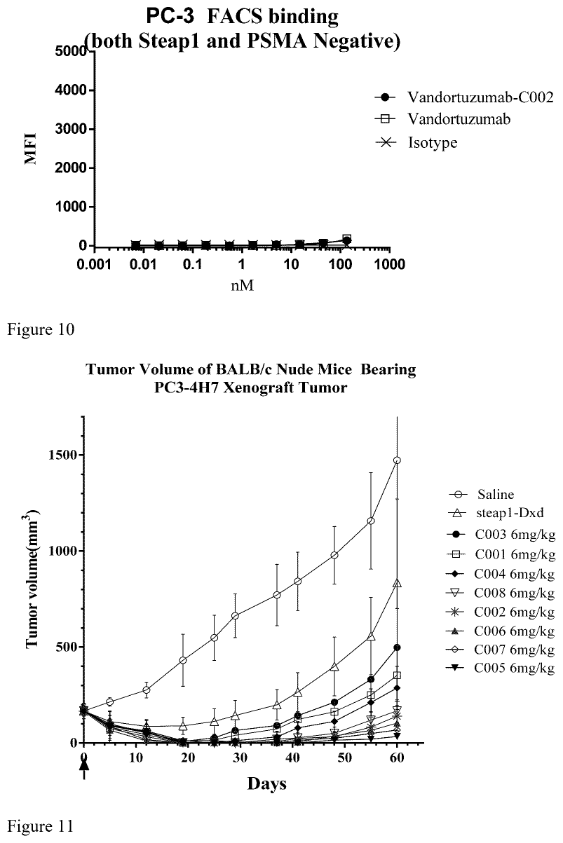

As data indicated in FIGS. 3 and 4, and in Table 3, when DAR was control at ˜6.0 by homogeneous conjugation, the drug conjugation sites in Steap1-A002 can be identified through using liquid chromatography-mass spectrometry (LC-MS^{E}). The primary drug conjugation sites of Steap1-A002 ADC were almost exclusively located on the cysteine residues SC (227) DK of the heavy and GEC (220) of the light chain and the cysteine residue THTC₍₂₃₃₎PPCPAPELLGGPSVFLFPPKPK in (the upper level of) the hinge region peptide segment.

TABLE 2
Mass Information for the Identification of Drug-Conjugated Sites
in a Steap1-ADC at DAR = 4.0

| | Observed | Observed | Expected | Mass error | |
|---|---|---|---|---|---|
| Peptide | RT (min) | [symbol] (Da) | mass (Da) | (ppm) | Response |
| SC₍₂₂₇₎DK | 53.32 | 2057.8832 | 2057.88487 | −0.8 | 73590360 |
| GEC₍₂₂₀₎ | 58.34 | 1913.7976 | 1913.79499 | 1.4 | 60442488 |

[symbol]

indicates data missing or illegible when filed

TABLE 3
Mass Information for the Identification of Drug-Conjugated
Sites in a Steap1-ADC at DAR = 5.95

| | Observed RT | Observed mass | Expected | Mass | |
|---|---|---|---|---|---|
| Peptide | (min) | (Da) | mass (Da) | error (ppm) | Response |
| SC₍₂₂₇₎DK | 54.01 | 2057.8808 | 2057.88487 | −2 | 150650496 |
| GEC₍₂₂₀₎ | 58.98 | 1913.8011 | 1913.79499 | 3.2 | 95556216 |
| THTC₍₂₃₃₎PPCP | 73.11 | 4393.1829 | 4393.13998 | 9.8 | 655808192 |
| APELLGGPSV | | | | | |
| FLFPPKPK | | | | | |

### Experiment 419: In Vitro Efficacy of Antibody-Drug Conjugates

The in vitro efficacies of the ADCs were compared with efficacy of antibody-GGFG-Dxd. In vitro cytotoxicities of DLL3, PSMA, cMet, Muc1, Guyc2C (GCC), Her2 bispecfic (trastuzumab/pertuzumab), EGFR/cMet, EGFR/Muc1, cMet/PD-L1, Mesothelin, 5T4, CDH6, CDH17, CD54, Tissue Factor (TF), B7H3, and PSMA/B7H3 ADCs at DAR=4.0±0.4 with the ADC structures of of this patent application: 30, 41, 55, 66, 76, 91, O-83, O-92, 106, 108, 126, 128, 134, 142, 144, 150, 172, 183, 187, 230, 259, 262, 275, 293, 308, 312, 314, 320, 328, 339, 349, 353, 360, 368, 375, 382, 385, 388, 393, 395, 405, 441, 449, C-484, C-488, C-490, C-491, C-495, C-496, C-497, C-499, C-500, C-501, C-502, C-503, C-504, C-505, C-506, C-507, C-508, C-509, C-510, C-511, C-512, C-513, C-514, C-515, C-516, C-517, C-518, C-519, C-520, C-521, C-522, C-523, C-524, C-525, C-526, C-527, C-528, C-529, C-530, C-531, C-532, C-533, C-534, C-535, C-536, C-537, C-538, C-539, C-540, C-541, C-542, C-543, C-544, C-545, C-546, C-547, C-548, C-549, C-550, C-552, C-570, C-571, C-573, C-574, C-575, C-576, C-577, C-578, C-579, C-580, C-581, C-582, C-583, C-584, C-585, C-586, C-587, C-589, C-590, C-591, C-592, C-593, C-594, C-595, C-596, C-597, C-598, C-599, C-600, C-601, C-602, C-603, C-604, C-605, C-606, C-607, C-608, C-609, C-610, C-611, C-612, C-613, C-614, C-615, C-616, C-617, C-618, C-619, C-620, C-621, C-622, C-623, C-624, C-625, C-626, C-627, C-628, C-629, C-630, C-631, C-632, C-633, C-634, C-635, C-636, C-637, C-638, C-639, C-640, C-641, C-642, C-643, C-644, C-645, C-646, C-647, C-648, C-649, C-650, C-651, C-652, C-653, C-654, C-655, C-656, C-657, C-658, C-659, C-660, C-661, C-662, C-663, C-664, C-665, C-666, C-667, C-668, C-669, C-670, C-671, C-672, C-673, C-674, C-675, C-676, C-677, C-678, C-679, C-680, C-681, C-682, C-683, C-684, C-685, C-686, C-687, C-688, C-689, C-690, C-691, C-692, C-693, C-694, C-695, C-696, C-697, C-698, C-001, C-002, C-003, C-004, C-005, C-006, C-007, C-008, C-009, C-010, C-011, C-012, C-013, C-014, C-015, C-016, C-017, C-018, C-019, C-020, C-021, C-022, C-023, C-024, C-025, C-026, C-027, C-028, C-029, C-030, C-031, C-032, C-033, C-034, C-035, C-036, C-037, C-038, C-039, C-040, C-041, C-042, C-043, C-044, C-045, C-046, C-047, C-048, C-049, C-050, C-051, C-052, C-053, C-054, C-055, C-056, C-057, C-058, C-059, C-060, C-061, C-062, C-063, C-064, C-065, C-066, C-067, C-068, C-069, C-070, C-071, C-072, C-073, C-074, C-075, C-076, C-077, C-078, and C-079. Their in vitro cytotoxicities were also evaluated in comparison with the Dxd-GGFG conjugates (DAR=4.0±0.4) of the same antibodies accordingly and along with Paclitaxel if needed as a control. Since the nucleoside antimetabolites and the other functional molecules analogs are 50 to over 1000 times of less potent than the cytotoxic CPT compounds or Tubulysin analogs or eribulin compounds in the application, thus the DAR is only counted the potent cytotoxic compounds in the ADC conjugates when the other small molecules of this patent are conjugated in the conjugates.

The cell lines used in the cytotoxicity assays were: C4-2B Cells which was obtained from ATCC through license agreement with MD Anderson Cancer Center, Houston, TX, USA. 22RV1, A549, U87, NCI-N87, MKN-7, BxPC-3, JIMT-1, MDA-MB-231, SU86.86, SW1990, OVCAR-3, Calu-3, A431, AsPC-1, CFPAC-1, DMS53, HCC827 and NCI-H₁₉₇₅ cells were purchased from ATCC, Nanjing Cobioer and The Cell Bank of Shanghai Institute of Biochemistry and Cell Biology, and the others. C4-2B and PC-3-4H7 are both Steap1 and PSMA antigen high express cells, but medium expressed of both B7H3 and DLL3 antigens. 22RV1 is low express cells for Steap1, PSMA, B7H3 and DLL3 antigens. U87 is glioblastoma (GBM) cells, NCI-N87 and MKN-7 cell line are stable models of the gastric epithelium. A431 is human epidermoid carcinoma. JIM-1 is trastuzumab-resistant Her2 breast tumor cells. SU86.86, SW1990, CFPAC-1 and AsPC-1 are pancreatic cancer cell lines. OVCAR-3 is ovarian cancer cell lines. Calu-3, HCC827 and NCI-H1975 are a human lung adenocarcinoma. All these cells were grown according to the provider manuals. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/mL) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation:

$$inhibition \% = [ 1 - ( assay - blank ) / ( control - blank ) ] \times 100.$$

TABLE 4
Proliferation-inhibiting effects of Anti-STEAP1 ADCs in C4-2B cell line.

| Drug | IC50 (nM) Mean |
|---|---|
| C001 (n = 4.2) | 47.71 |
| C002 (n = 4.2) | 6.23 |
| C003 (n = 4.2) | 10.62 |
| C004 (n = 4.3) | 12.88 |
| C005 (n = 4.1) | 1.53 |
| C006 (n = 4.3) | 4.84 |
| C007 (n = 4.2) | 3.57 |
| C008 (n = 4.3) | 1.14 |
| C009 (n = 4.1) | 2.57 |
| C010 (n = 4.3) | 0.89 |
| C011 (n = 4.2) | 1.47 |
| C012 (n = 4.3) | 1.08 |
| C013 (n = 4.1) | 6.83 |
| C014 (n = 4.3) | 1.63 |
| C015 (n = 4.2) | 1.77 |
| C016 (n = 4.3) | 1.54 |
| C017 (n = 4.3) | 1.41 |
| C018 (n = 4.1) | 7.41 |
| C019 (n = 4.3) | 8.39 |
| C020 (n = 4.2) | 7.87 |
| C021 (n = 4.2) | 7.93 |
| C022 (n = 4.3) | 9.18 |
| C023 (n = 4.1) | 9.85 |
| C024 (n = 4.3) | 11.61 |
| C025 (n = 4.2) | 10.73 |
| C026 (n = 4.3) | 9.54 |
| C027 (n = 4.1) | 12.43 |
| C028 (n = 4.3) | 7.39 |
| C029 (n = 4.2) | 7.81 |
| C030 (n = 4.2) | 7.27 |
| C031 (n = 4.2) | 6.53 |
| C032 (n = 4.3) | 6.38 |
| C033 (n = 4.1) | 6.65 |
| C034 (n = 4.3) | 17.81 |
| C035 (n = 4.2) | 11.76 |
| C036 (n = 4.3) | 6.54 |
| C037 (n = 4.1) | 4.45 |
| C038 (n = 4.3) | 7.19 |
| C039 (n = 4.2) | 5.84 |
| C040 (n = 4.2) | 4.27 |
| C041 (n = 4.2) | 3.57 |
| C042 (n = 4.3) | 8.38 |
| C043 (n = 4.1) | 7.65 |
| C044 (n = 4.3) | 7.87 |
| C045 (n = 4.2) | 6.76 |
| C046 (n = 4.2) | 7.47 |
| C047 (n = 4.2) | 5.53 |
| C048 (n = 4.3) | 6.78 |
| C049 (n = 4.1) | 5.95 |
| C050 (n = 4.3) | 7.31 |
| C051 (n = 4.2) | 7.72 |
| C052 (n = 4.3) | 7.14 |
| C053 (n = 4.1) | 6.35 |
| C054 (n = 4.3) | 6.19 |
| C055 (n = 4.2) | 5.34 |
| C056 (n = 4.2) | 13.27 |
| C057 (n = 4.2) | 10.57 |
| C058 (n = 4.3) | 6.68 |
| C059 (n = 4.1) | 5.55 |
| C060 (n = 4.3) | 7.57 |
| C061 (n = 4.2) | 6.16 |
| C062 (n = 4.3) | 5.14 |
| C063 (n = 4.1) | 4.75 |
| C064 (n = 4.3) | 5.69 |
| C065 (n = 4.2) | 7.14 |
| C066 (n = 4.2) | 3.89 |
| C067 (n = 4.2) | 3.57 |
| C068 (n = 4.3) | 3.48 |
| C069 (n = 4.1) | 6.55 |
| C070 (n = 4.3) | 12.57 |
| C071 (n = 4.2) | 6.56 |
| C072 (n = 4.1, n1 = 5) | 7.95 |
| C073 (n = 4.3) | 6.27 |
| C074 (n = 4.2) | 7.66 |
| C075 (n = 4.1, n1 = 5) | 6.75 |
| C707 (n = 4.3) | 8.43 |
| C708 (n = 4.2) | 7.86 |
| C709(n = 4.2) | 1.46 |
| C710(n = 4.1, n1 = 5) | 13.75 |
| C711 (n = 4.3) | 0.73 |
| C712 (n = 4.2) | 10.89 |
| C713 (n = 4.2) | 9.13 |
| STEAP1-Dxd (n = 7.4) | 76.87 |

Table 5 Proliferation-inhibiting effects of B7H3 ADCs in 22RV1 cell line.

### Experiment 420: In Vivo Efficacy and Safety of Antibody-Drug Conjugates

Example 669. Antitumor Activities in vivo (BALB/c Nude Mice Bearing PC3-4H7, 22RV1 or C4-2B cells for Steap1-ADCs and B7H₃-ADCs, or bearing Calu 6 and HCC827 cells for B7H₃-ADC; JIMT-1, CFPAC, SU86.86, or SW1990 cells for Her2 bispecific-ADCs, EGFR/Muc1 or Muc1-ADCs; or bearing Calu 6 and HCC827 cells for B7H₃-ADC; NCI-HCC827, or MDA-MB-231, OVCAR-3, HCC827, A431 or H1975 cells for AXL-ADCs, 5T4-ADC, Folate-ADCs, cMet/PD-L1 ADC, or EGFR-ADCs; Xenograft Tumors independently).

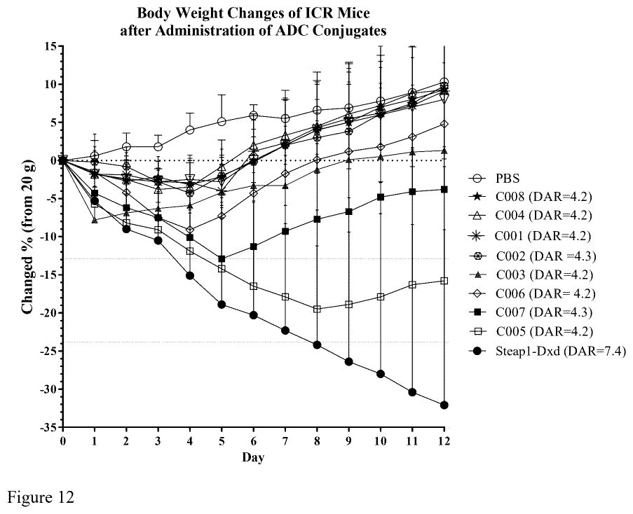

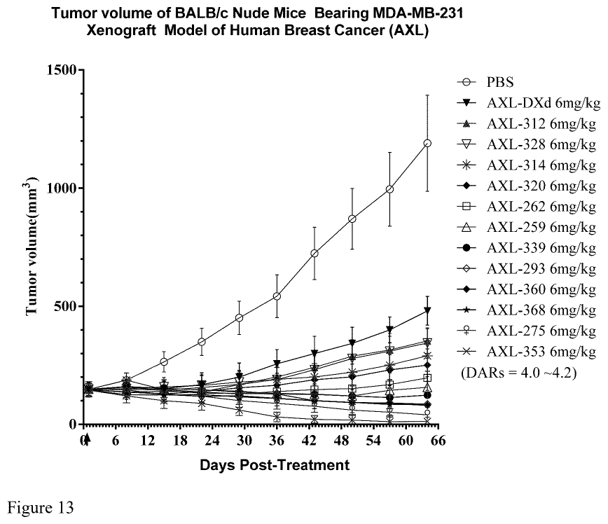

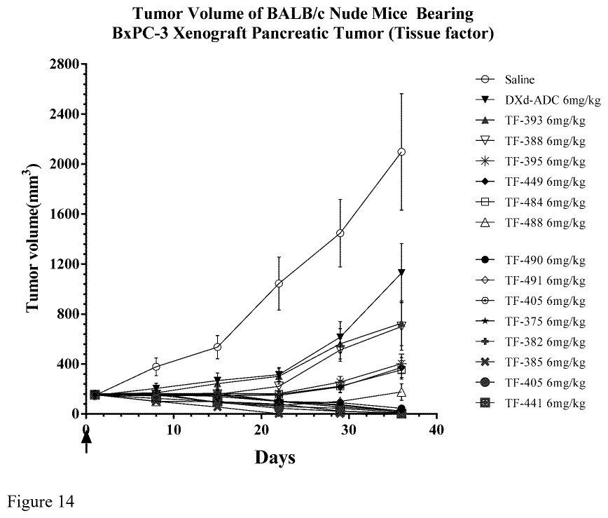

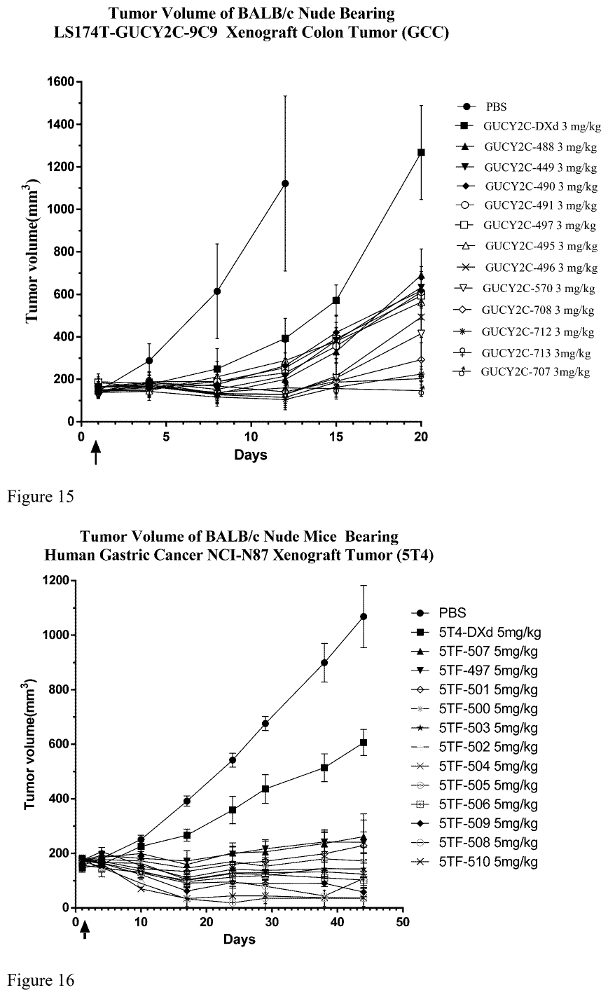

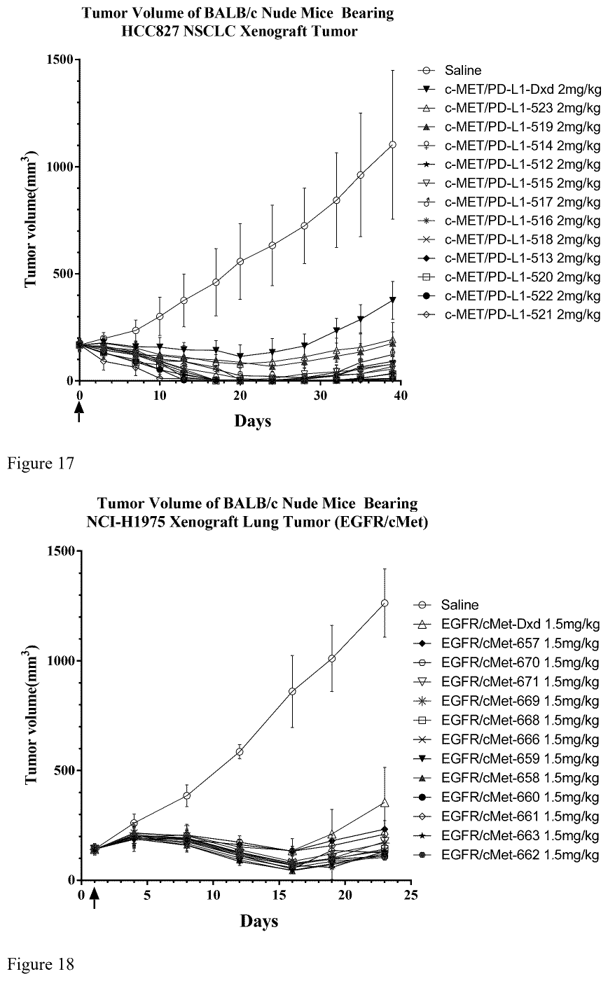

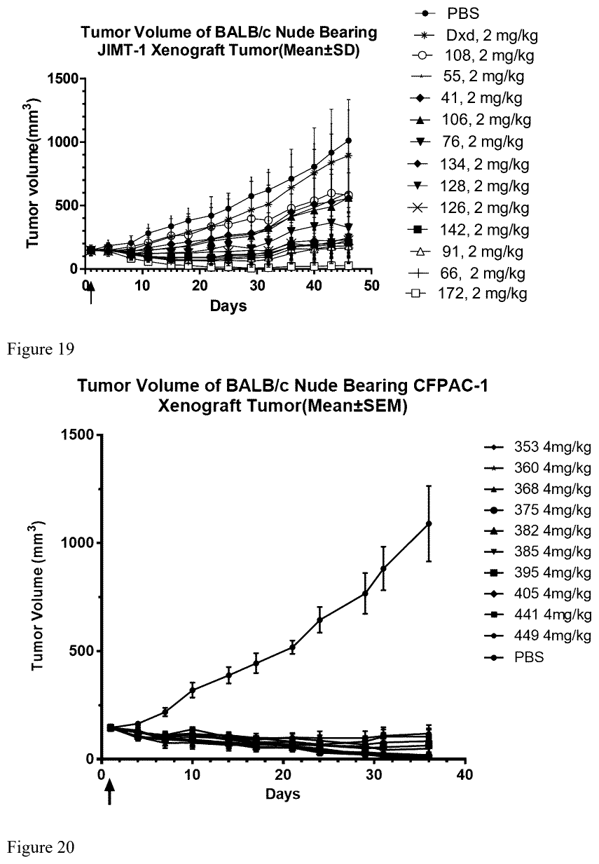

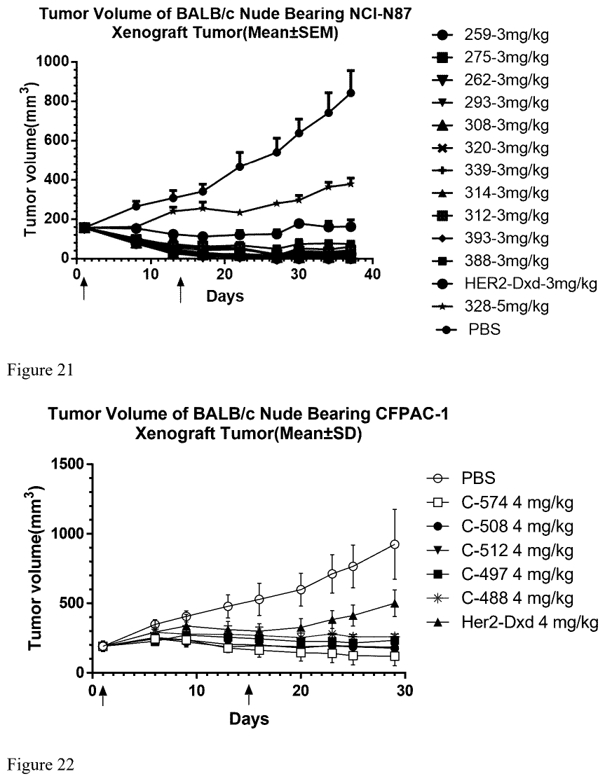

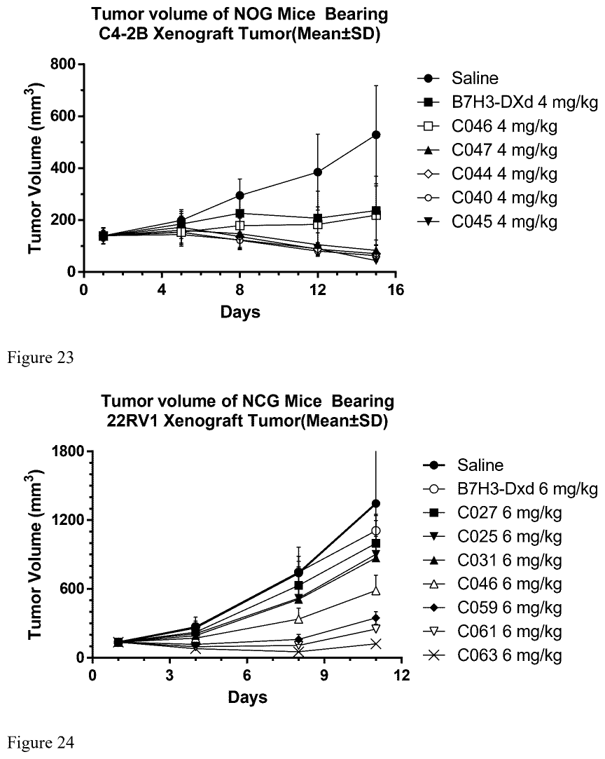

The in vivo efficacy of conjugates of against tumor cells, in xenograft models were exampled in FIGS. 11-24. Five-week-old female BALB/c Nude mice (6 animals per group) were inoculated subcutaneously in the area under the right shoulder with respective carcinoma cells (5×10⁶ cells/mouse) in 0.1-0.2 mL of serum-free medium. The tumors were grown for 6-35 days to an average size of 150 mm³, or 8-40 days to an average size of 180 mm³. The animals were then randomly divided into different groups (6 animals per group). The first group of mice served as the control group and was treated with the phosphate-buffered saline (PBS) vehicle. The other groups were treated with conjugates at doses 1.0˜8.0 mg/Kg (some of them were described in the figures), administered only once intravenously. If a control of paclitaxel was used, it was administrated at dose of 15 mg/Kg, once a week for three weeks intravenously. Three dimensions of the tumor were measured every 3 or 4 days (twice a week) and the tumor volumes were calculated using the formula tumor volume=1/2×(length×width×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

The results of the examples were plotted in FIGS. 11-24. All the conjugates did not cause the animal body weight loss at the administrated doses or up to 300 mg/Kg. All conjugates demonstrated antitumor activity as comparison with PBS buffer, and most of the conjugates of invention showed better antitumor activities than the conjugates with the payload/linker complexes of GGFG-Dxd.

### Example 421. Pharmacokinetic Study in Mice

CD-1 mice were randomly assigned to each group (n=6) and were administered intravenously with each ADC at a dose of 10 mg kg. Blood samples (50 μL) were collected from each animal via the tail vein at each time point (5 min, 4 h, 1 day, 2 days, 3 days, 4 days, 7 days, 10 days, 14 days, 21 days, and 28 days). After removal of cells by centrifugation (10 min at 2200×g at 4° C.), Serum samples were stored at −80° C. until used for subsequent sandwich ELISA.

### Example 422. ELISA Assay Method to Measure Drug Concentration in Mouse Serum

For determination of the total antibody concentration (both conjugated and unconjugated), a 96-well plate (Corning) was coated with target protein (2 μg/ml, 100 μl per well) diluted in PBS. After overnight incubation at 4° C., the plate was blocked with 100 μL of 1% BSA in PBS containing 0.05% Tween 20 (PBS-T) at room temperature for 2 h. Subsequently, the solution was removed and each diluted serum sample was added to each well, and the plate was incubated at room temperature for 1 h. After each well was washed three times with PBS-T, 100 μL of goat anti-human IgG Fc-HRP conjugate (1:50,000) was added. After being incubated at room temperature for 1 h, the plate was washed and color development was performed by adding 100 μL per well TMB substrate to the plate. For determination of ADC concentration (conjugated only), assays were performed in the same manner using mouse anti-payload antibody for plate coating, biotinylated target protein (1:5000), and SA-HRP conjugate (1:120,000) as secondary and tertiary detection antibodies, respectively. All assays were performed in duplicate. Concentrations were calculated based on a standard curve.

1. An antibody drug conjugate with a branch functional small molecule having one of Formula (I), (II), (III) and (IV) represented as: 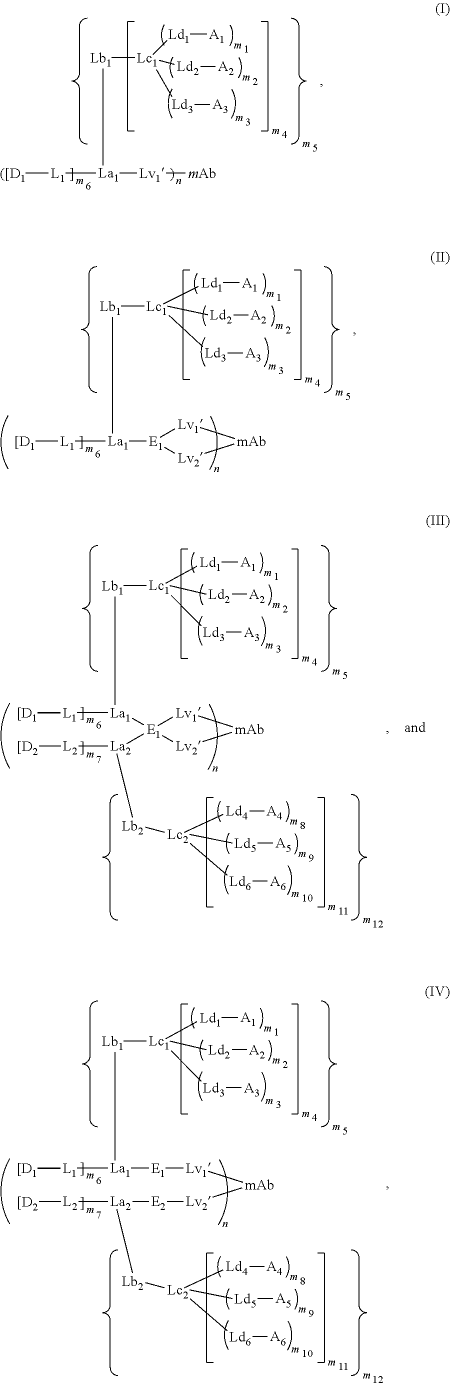
wherein,
D₁ and D₂ are a cytotoxic agent; mAb is an antibody or antibody like protein; n is 1, 2, 3, 4, 5, 6, 7, 8, 9, 10, 11, 12, 13, 14, 15, 16, 17, 18, 19 or 20, and these numbers may have a decimal;
L₁, L₂, La₁, La₂, Lb₁, Lb₂, Lc₁, Lc₂, Ld₁, Ld₂, Ld₃, Ld₄, Ld₅, and Ld₆ are a linker, which are independently selected from O, C, CH, CH₂, NH, S, N, NH—NH, N—N, N(R₃), N(R₃)N(R_{3′}), C(═O), C(═O)N, C(═O)NH, C(—O)N—N, C₂-C₈ of alkyl (alkylene), heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or 1˜8 natural or unnatural amino acids described in the definition; or polyethyleneoxy unit of formula (OCH₂CH₂)ₚOR₃, (OCH₂CH₂)ₚ, (OCH₂CH(CH₃))ₚOR₃, NH(CH₂CH₂O)ₚR₃, NH(CH₂CH(CH₃)O)ₚR₃, or N[(CH₂CH₂O)ₚR₃][(CH₂CH₂O)ₚR_{3′}], (OCH₂CH₂)ₚCOOR₃, CH₂CH₂(OCH₂CH₂)ₚCOR₃, or CH₂CH₂(OCH₂CH₂)ₚCOOR₃, wherein p and p′ are independently an integer selected from 0 to about 100, or combination thereof; wherein R₃ and R_{3′} are independently H, C(—O) H, C(═O)CH₃, NH, C(═O), C(═O)NH, C₁-C₈ of alkyl, absent; or combination above thereof; and one or several of L₁, L₂, La₁, La₂, Lb₁, Lb₂, Lc₁, Lc₂, Ld₁, Ld₂, Ld₃, Ld₄, Ld₅, and Ld₆ can be absent thereof;
or La₁ and La₂ are independently a peptide having 1˜8 amino acids, which contain one or several lysine, glutamic acid, aspartic acid, cysteine, glutamine, asparagine and tyrosine, and these amino acids can link A₁, A₂, A₃, A₄, A₅ and A₆ through Lb₁, Lb₂, Lc₁, Lc₂, Ld₁, Ld₂, Ld₃, Ld₄, Ld₅, and Ld₆ accordingly;
wherein Lv₁′ and Lv₂′ are independently or jointly having the following structures: 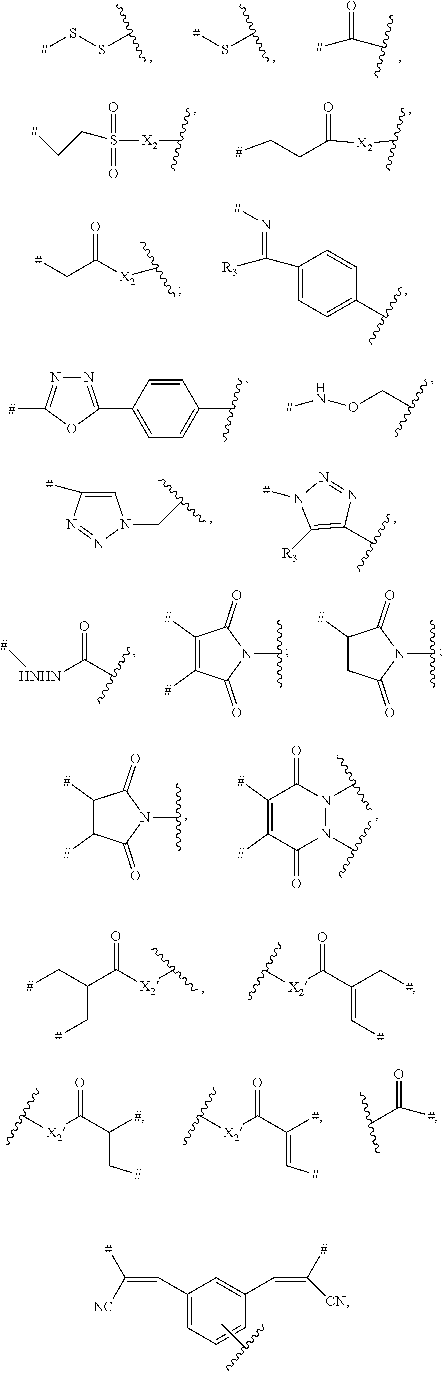 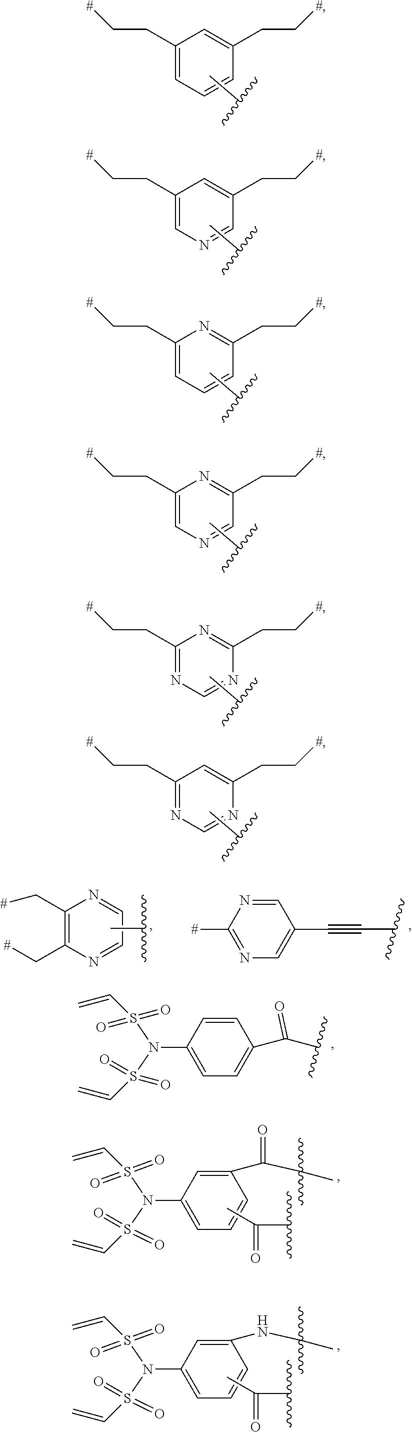 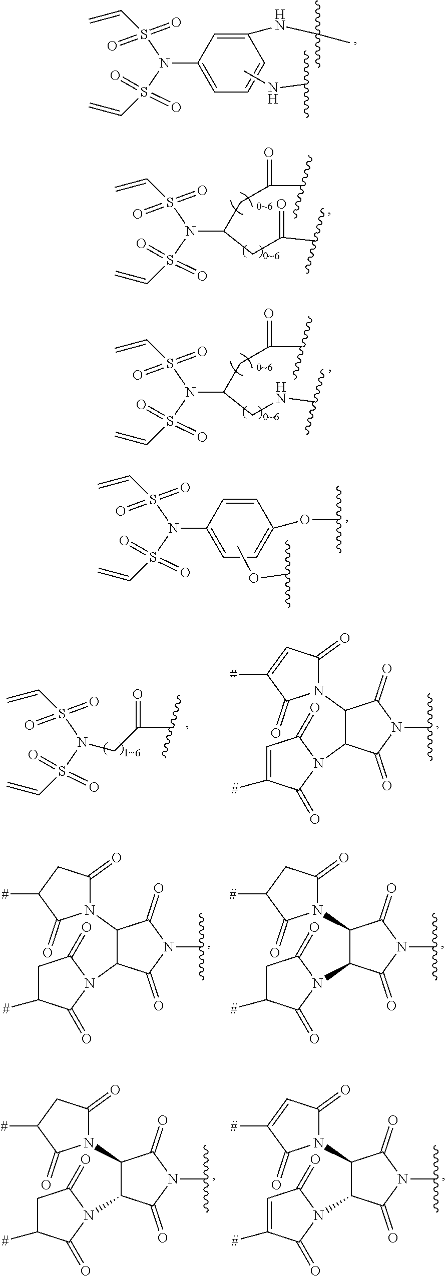 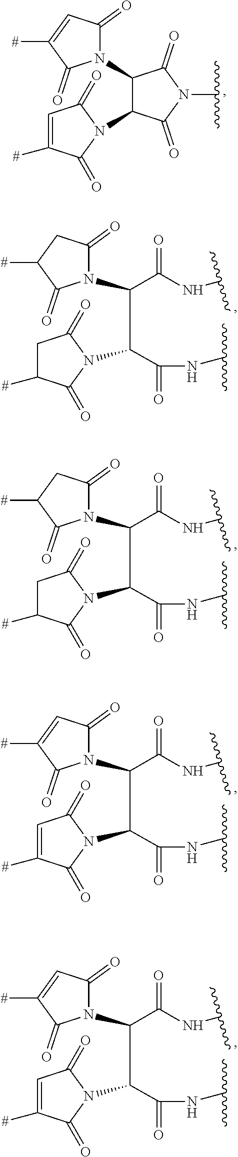 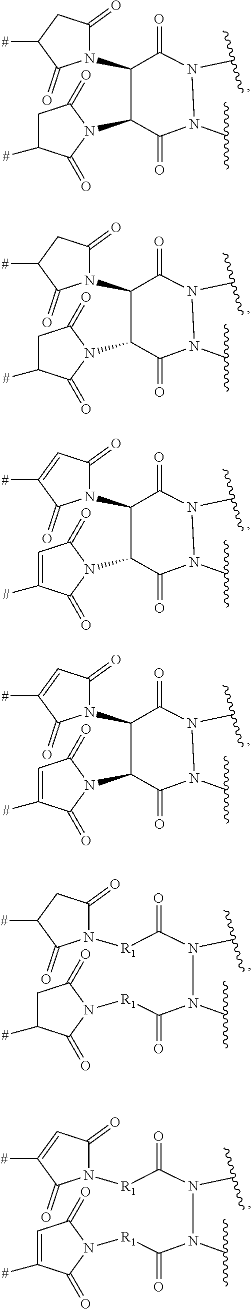 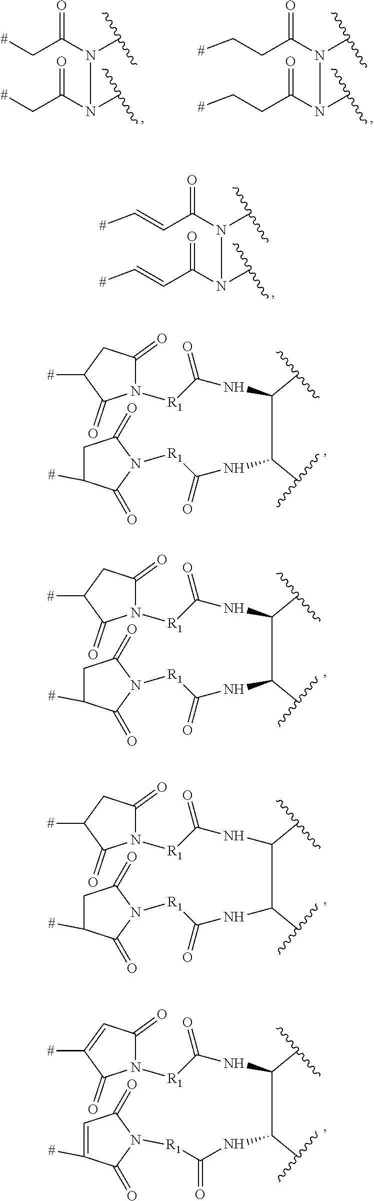 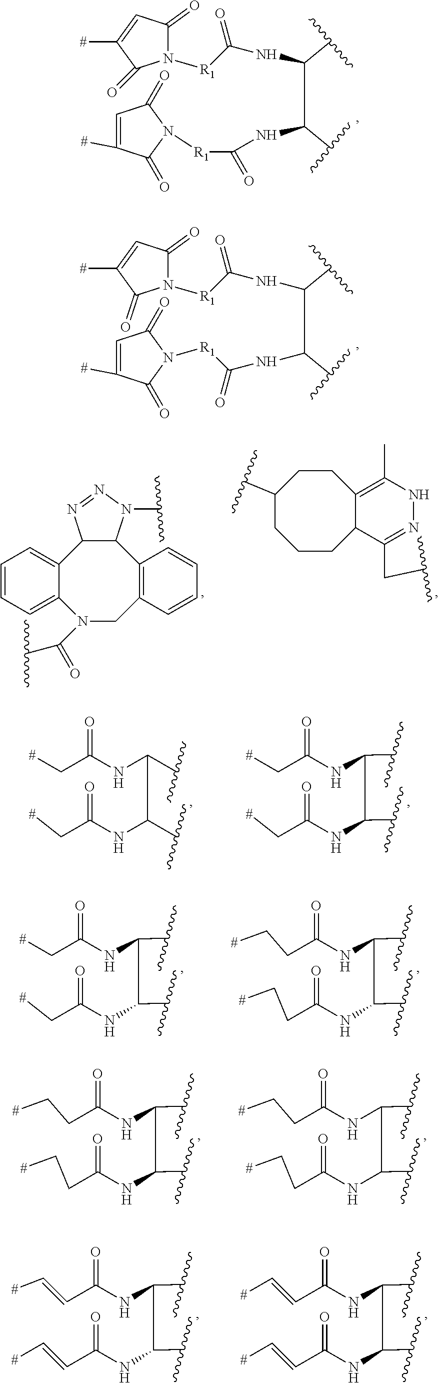 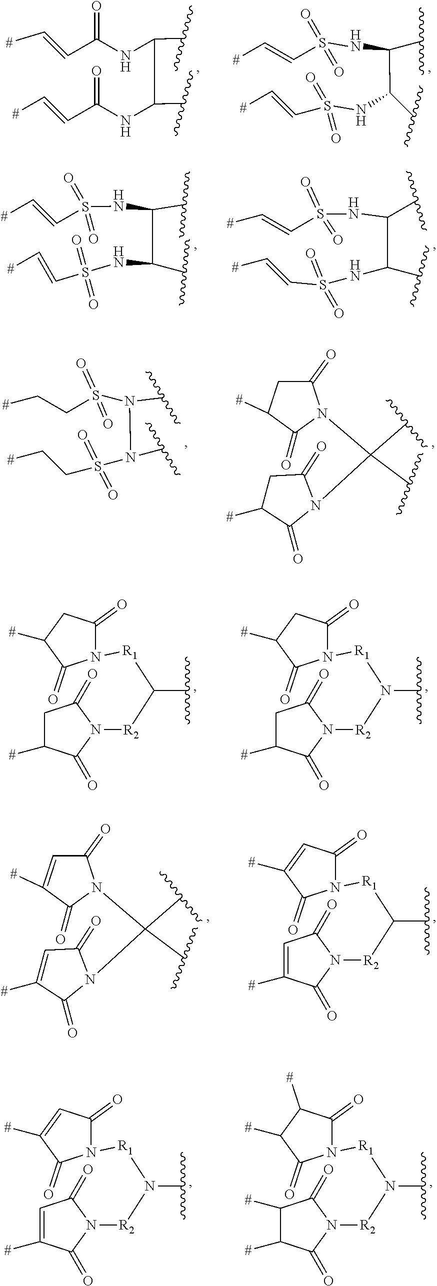 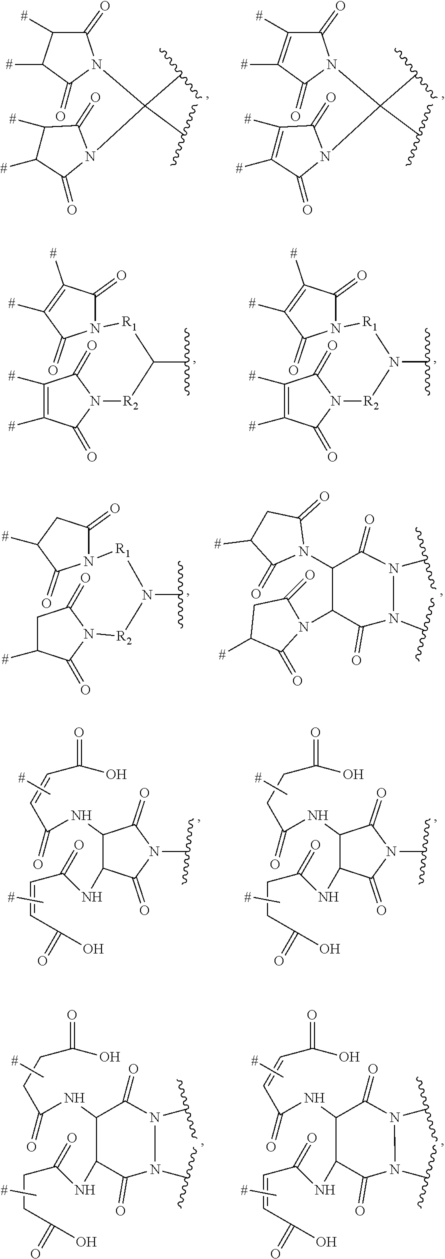 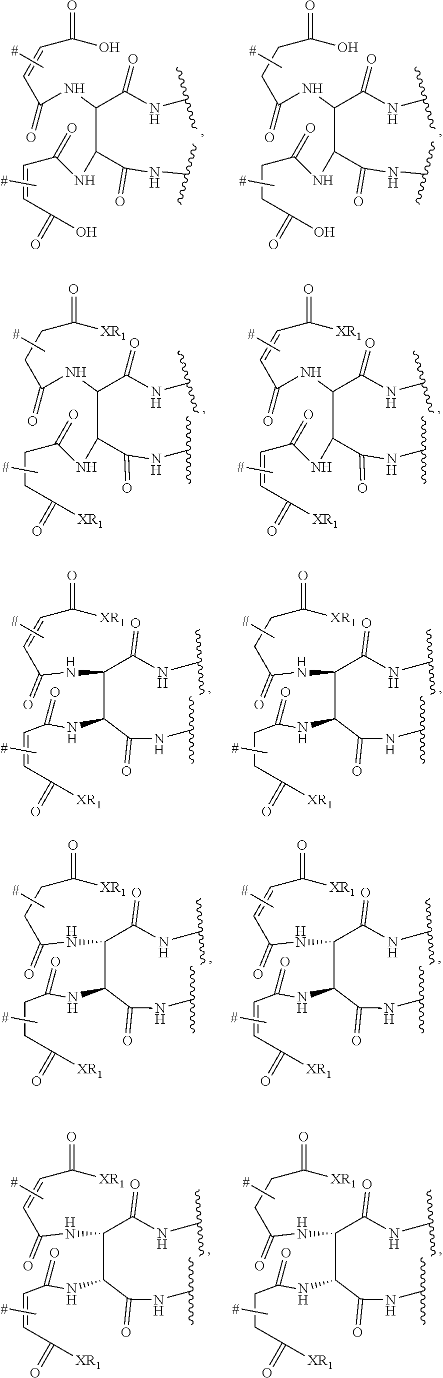 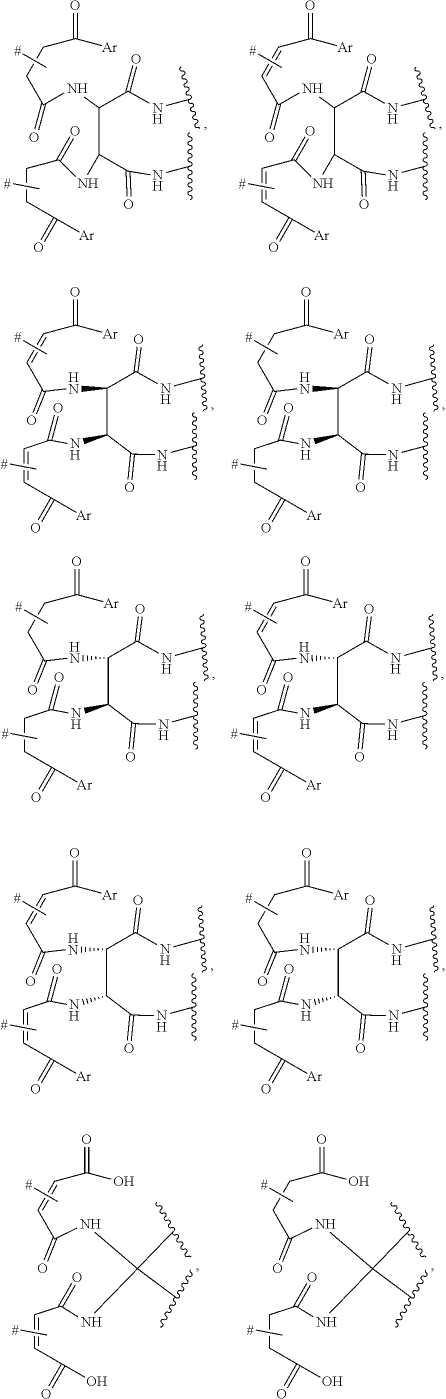 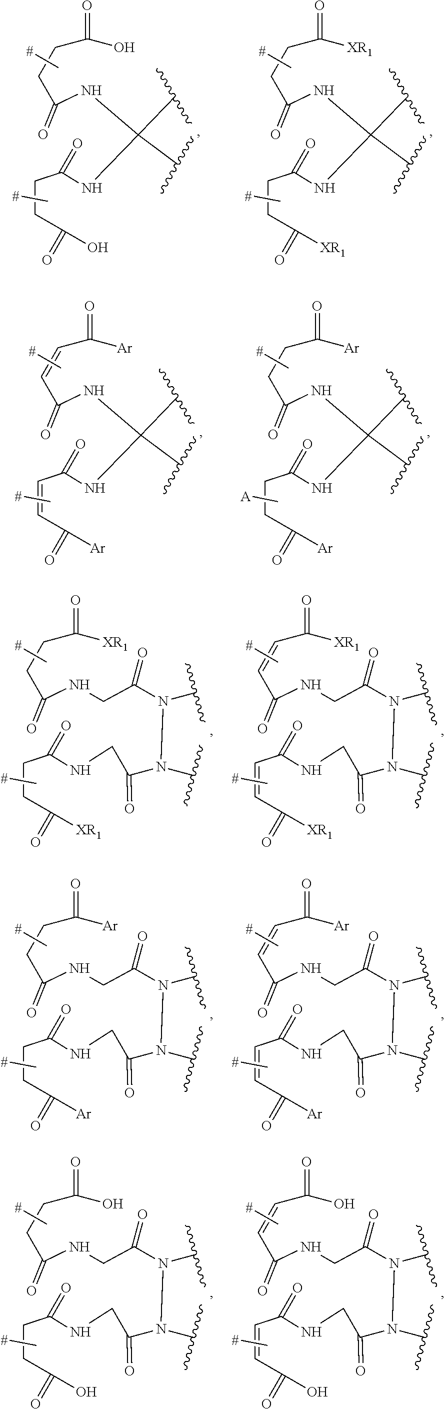 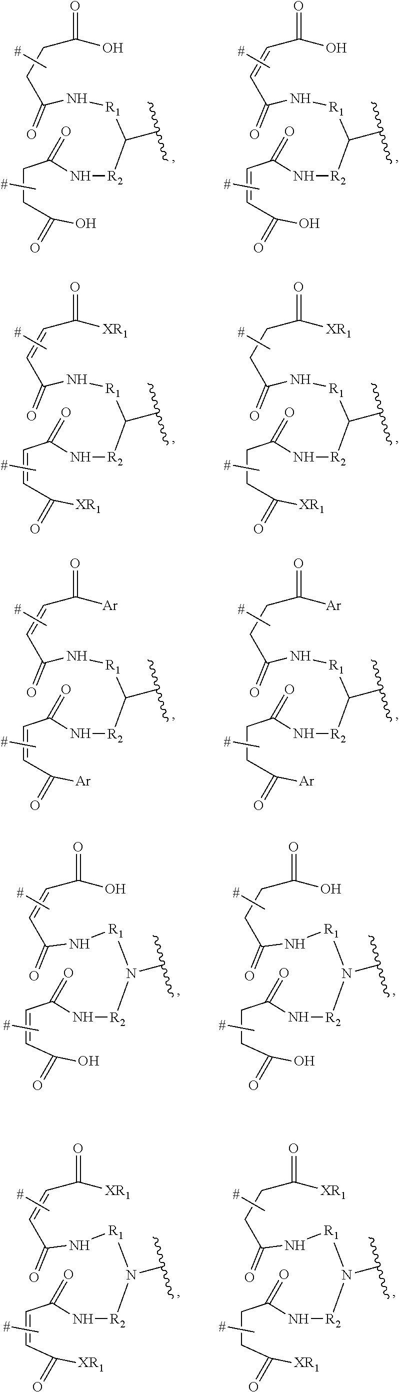 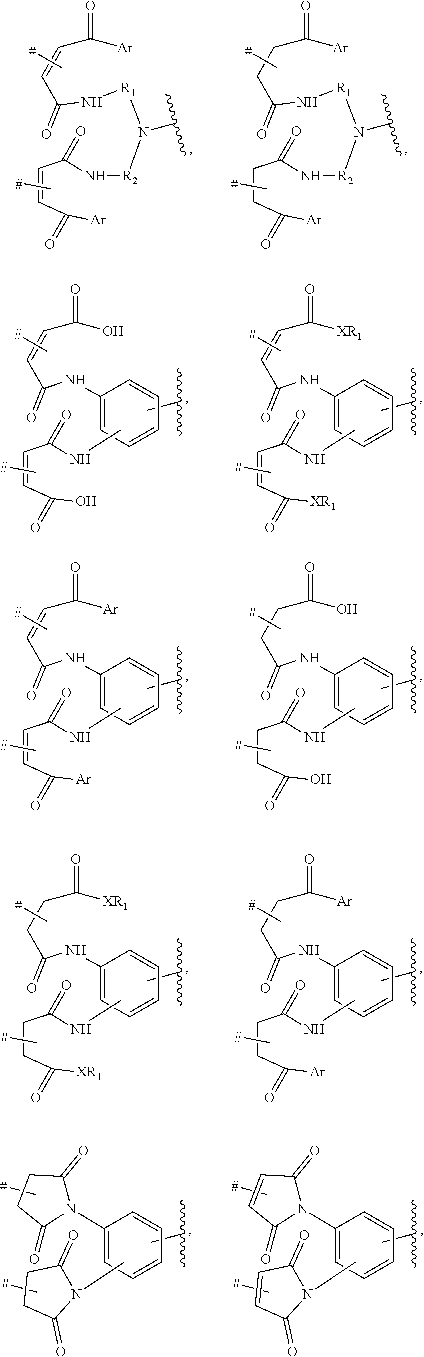 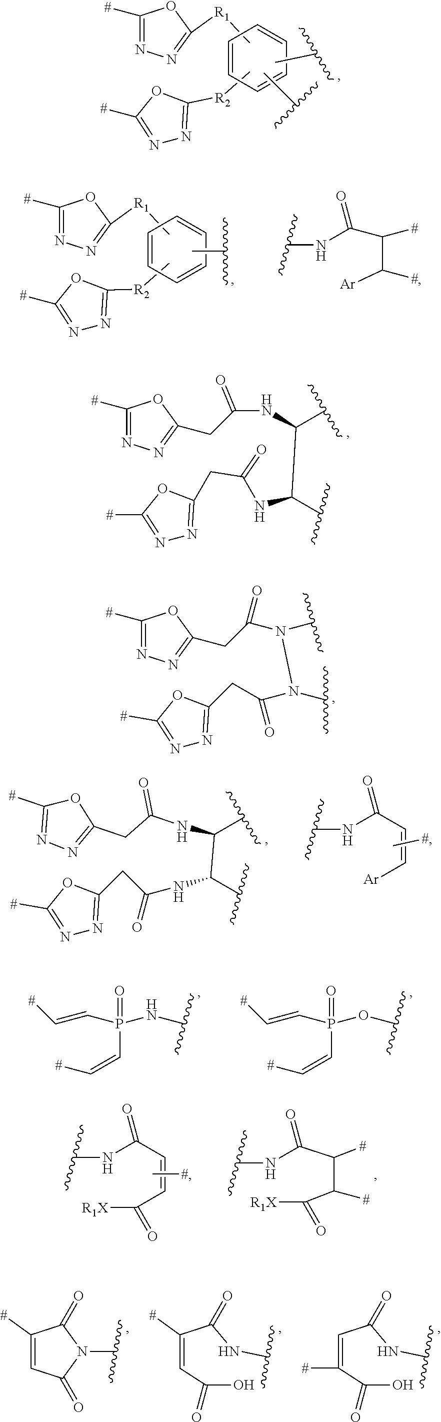 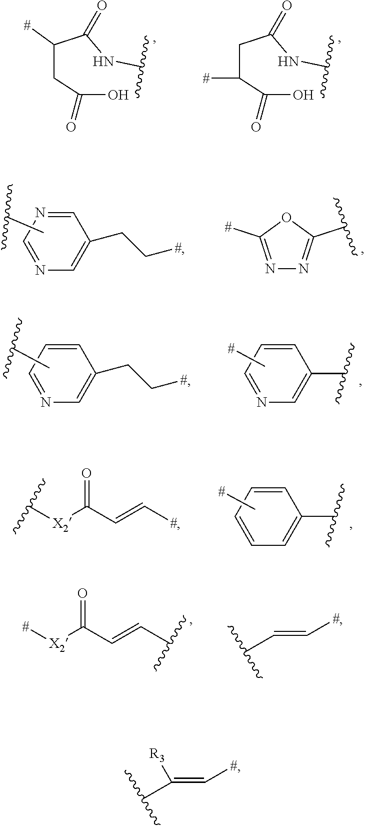
wherein “ [symbol] ” is a site that links a drug or a site of linker L₁ or L₂; “#” is a site that links a S (thiol), O (phenol), NH (amino), CHO (aldehyde), C(═O) (ketone), C(O)(NH) (amide) and C(O)(OH) (carboxylate) of an antibody; wherein R₁ and R₂ are H, C₁-C₆ of alkyl or a peptide containing 1˜4 units of aminoacids; X, X₁′ and X₂′ are O, NH, S, CH₂; the connecting bond “—” in the middle of the two atoms means it can link either one of the two atoms, Ar is an aromatic group;
E₁ and E₂ are independently selected from CH, CH₂, CH—CH, NH, NHNH, N(R₃), N(R₃) N(R_{3′}), N═N, N—N, P, P(═O), S, Si, C₂-C₈ of alkyl, heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; a peptide containing 1˜4 units of aminoacids, or one of the following structures: 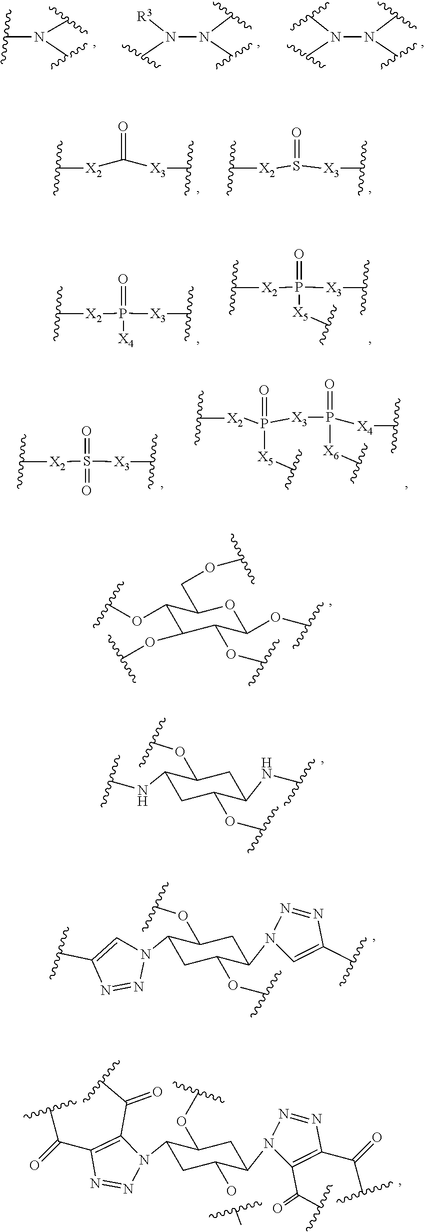 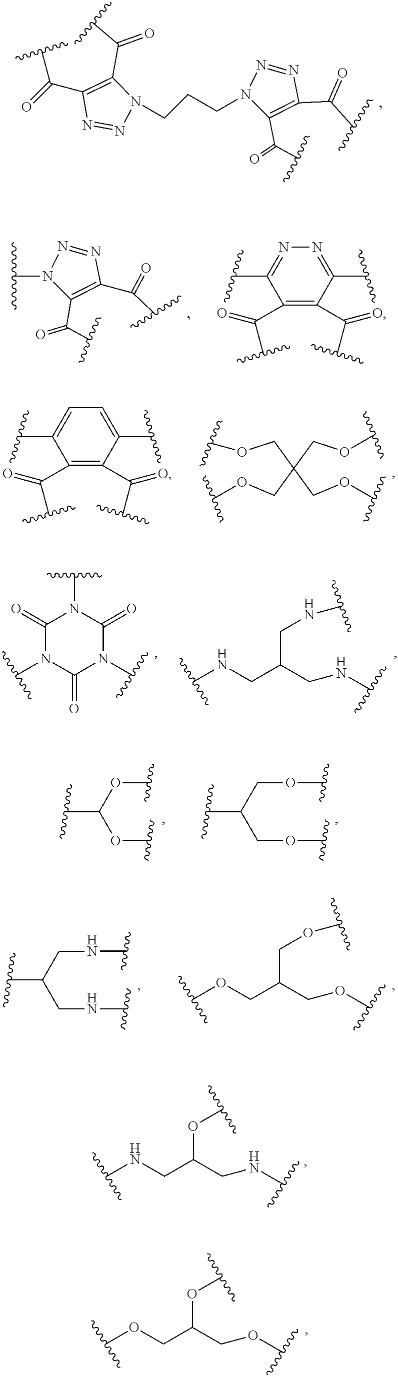 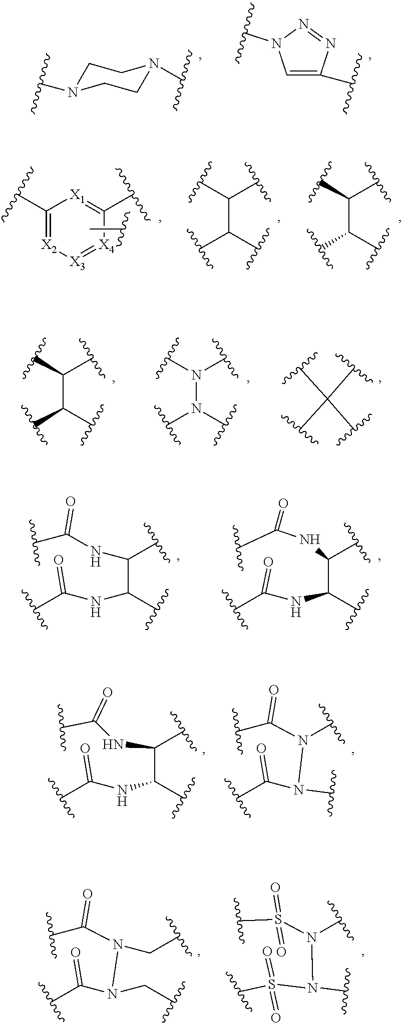
wherein [symbol] is the site of linkage; X₁, X₂, X₃, X₄, X₅, or X₆, are independently selected from NH; NHNH; N(R₃); N(R₃)N(R_{3′}); O; S; C₁-C₆ of alkyl; R₃ and R₃′ are H, C₁-C₆ of alkyl, or E₁ can be absent, thus La₁ or/and La₂ can directly link to Lv₁′ or Lv_{2′};
m₁, m₂, m₃, m₄, m₅, m₆, m₇, m₈, m₉, m₁₀, m₁₁ and m₁₂ are independently 1, 2, 3, 4, 5, 6, 7, 8, 9, or 10 and these numbers may have a decimal; or m₂, m₃, m₅, m₉ and/or m₁₀ can be 0, thus Ld₂-A₂, Ld₃-A₃, Ld₅-A₅, and/or Ld₆-A₆ can be absent;
A₁, A₂, A₃, A₄, As and A₆ are independently a functional small molecule selected from (1) an affinity ligand: cell-penetrating peptide (CPP), including a ligand for bombesin receptor/neurotensin receptor or neuropeptide-Y receptor; (2) and/or a nucleoside antimetabolite/analog, or/and a chemotherapy drug; which have either the synergy with D1 or D2, or enhanced affinity for mAb.

2. The antibody drug conjugate according to claim 1, wherein the functional small molecule, A₁, A₂, A₃, A₄, A₅ and A₆ are independently an affinity ligand, which is selected from: 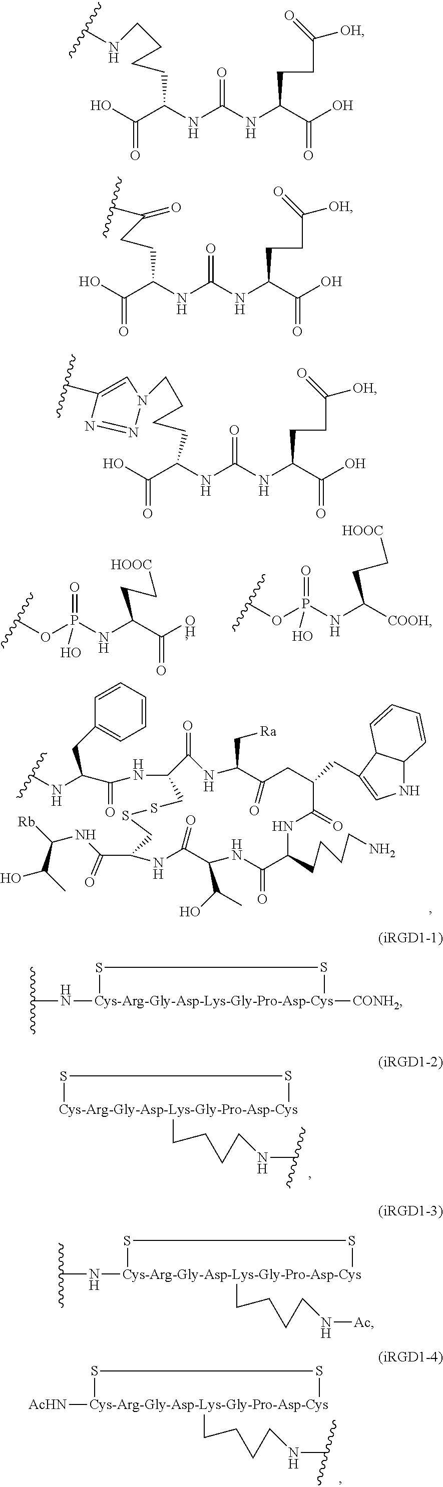 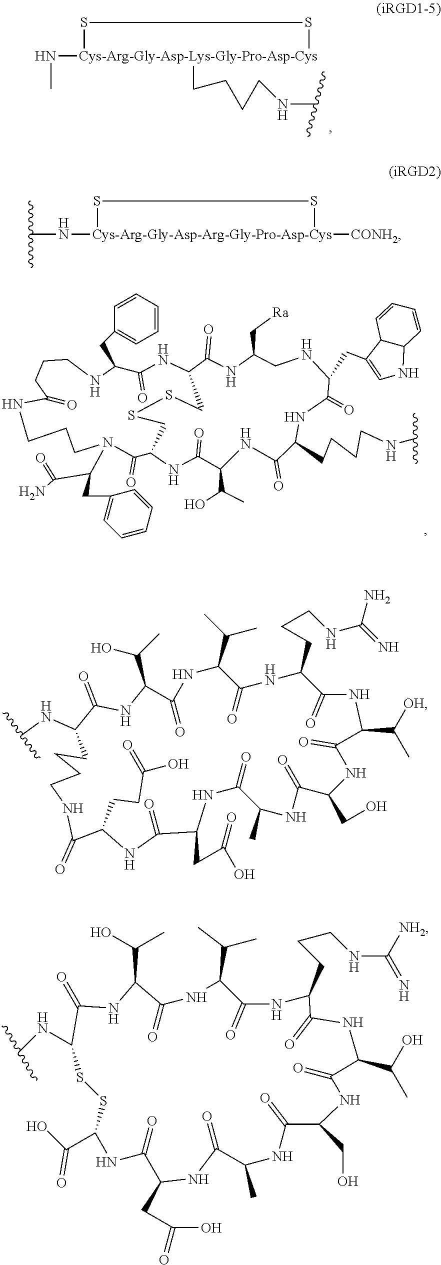 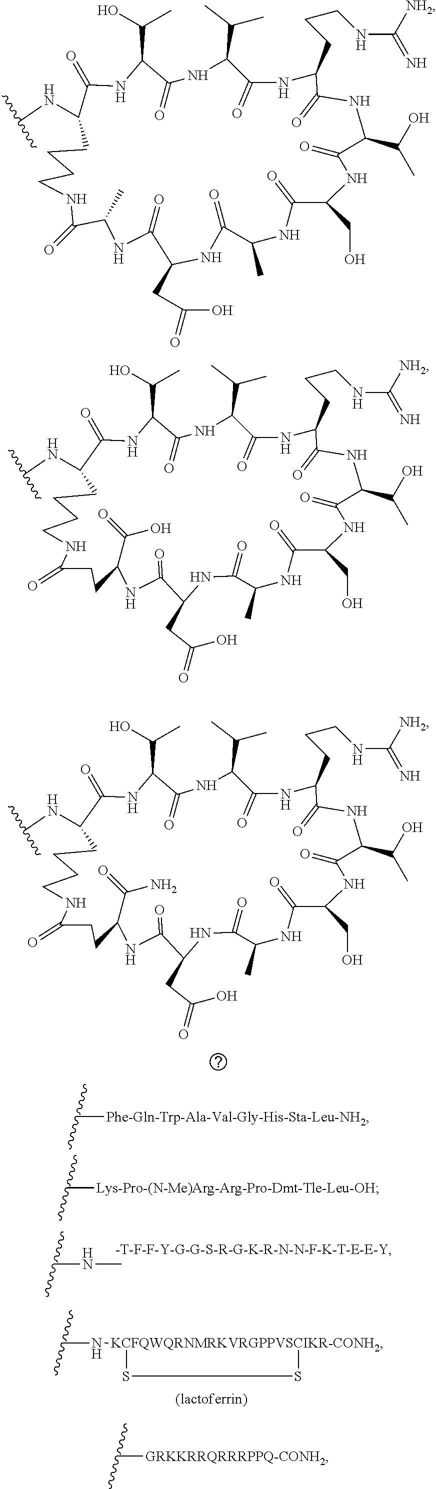 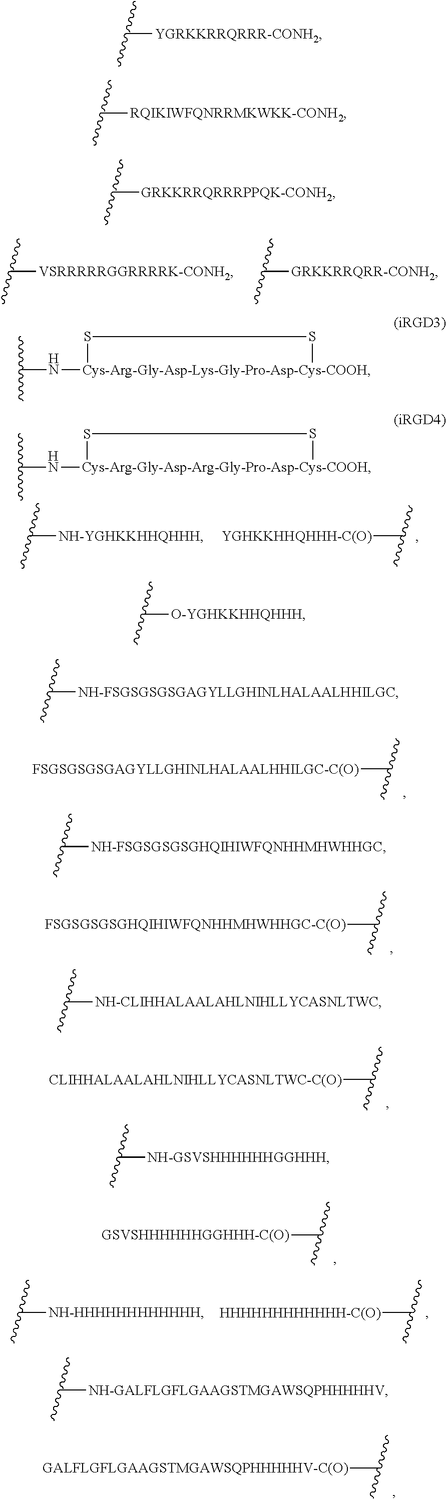 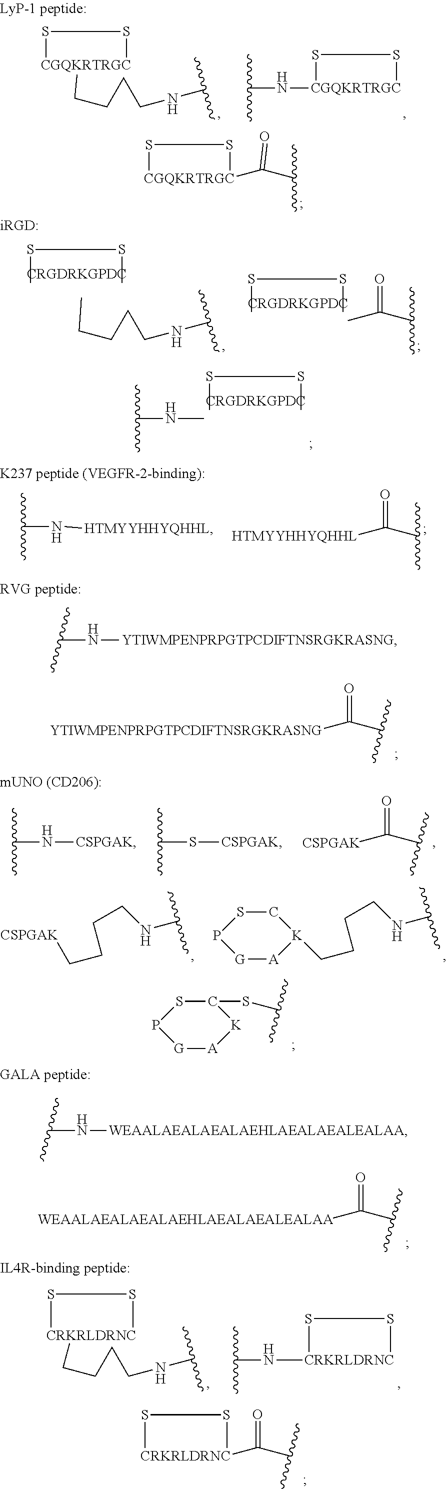 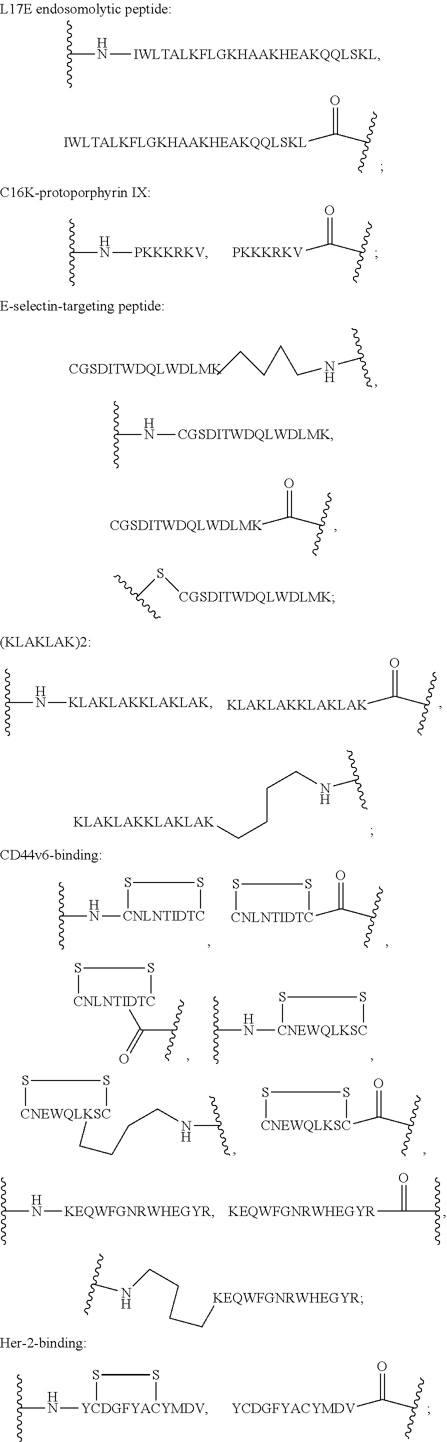 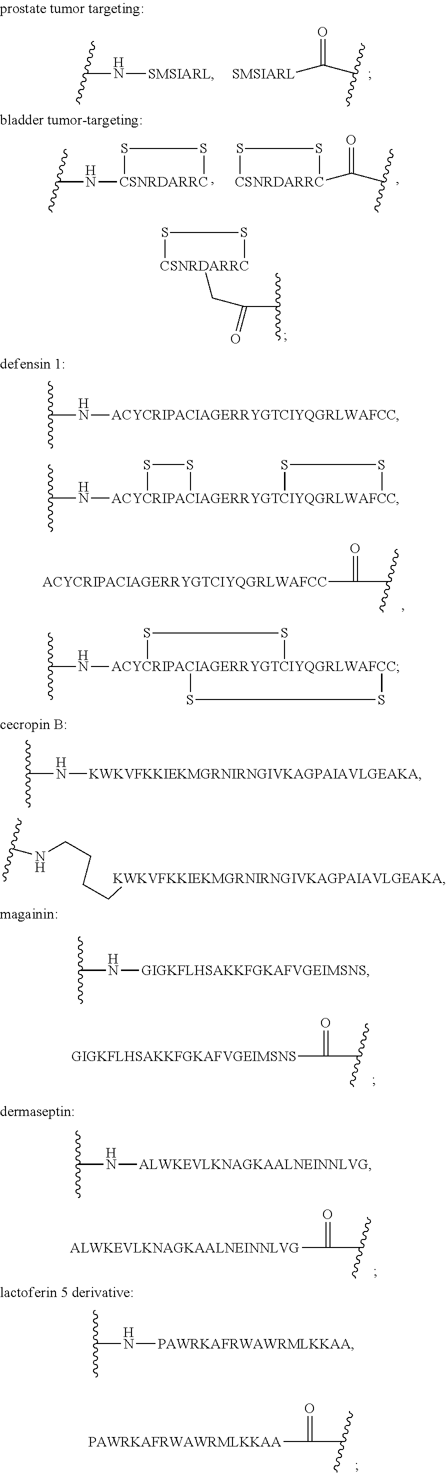 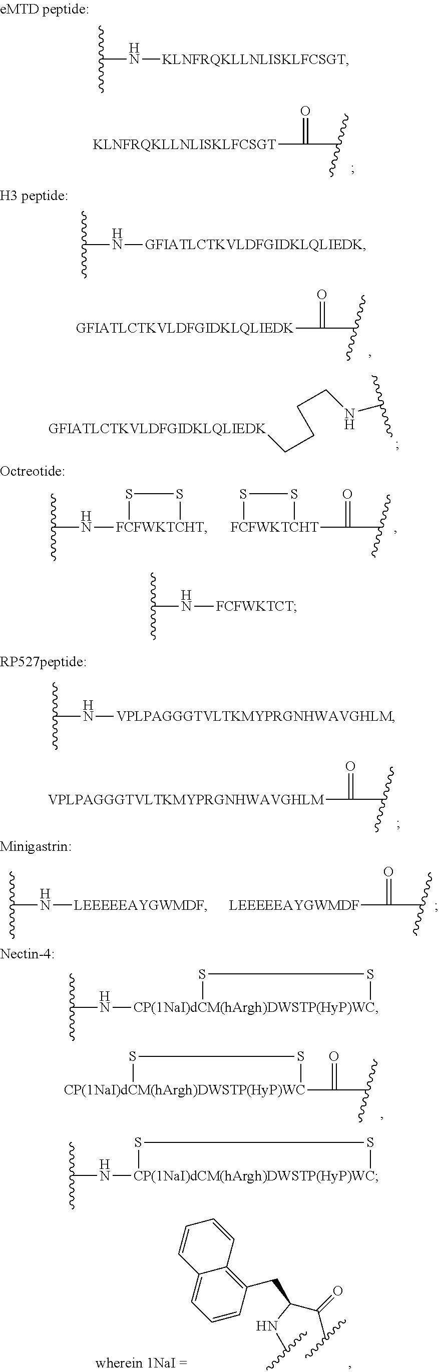 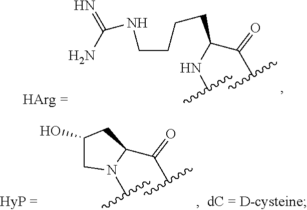 [SMILES: *N[C@@H](CCCCNC(=N)N)C(*)=O ; *C(=O)[C@@H]1C[C@@H](O)CN1*] 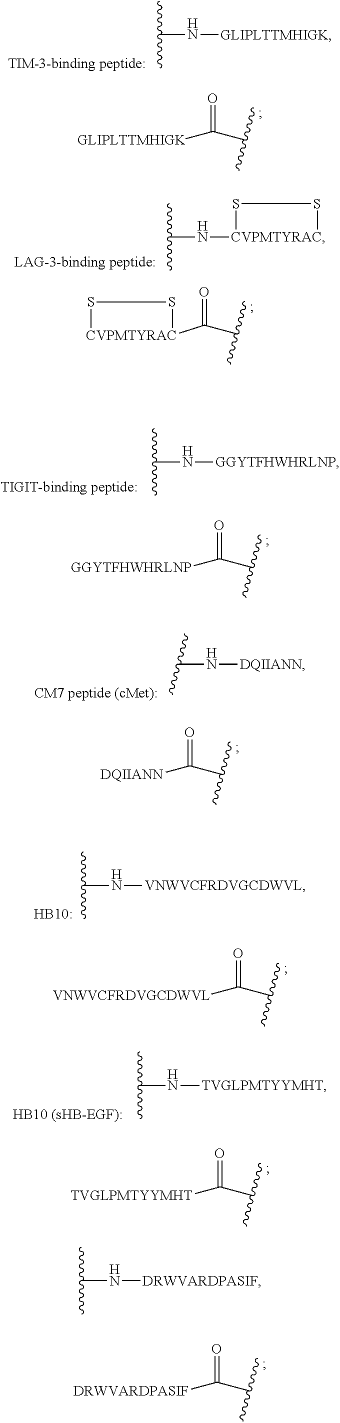 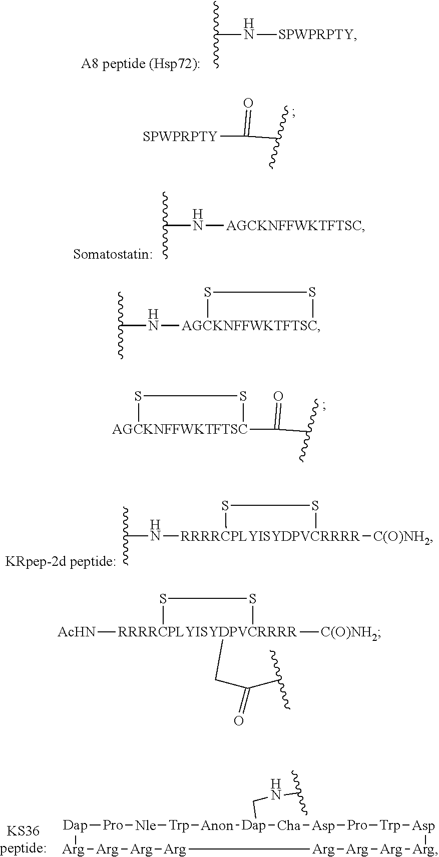
wherein Dap is(S)-2,3-Diaminopropanoic acid, Nle is L-Norleucine, Anon is(S)-2-Aminononanoic acid, Cha is L-Cyclohexylalanine. The others are natural amino acids. 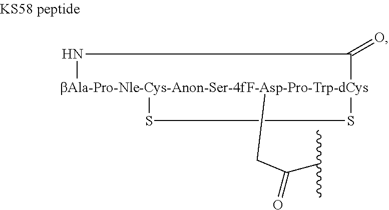
wherein βAla is Beta-alanine, Nle is L-Norleucine, Anon is(S)-2-Aminononanoic acid, 4fF is 4-Fluoro-1-phenylalanine, dCys is D-cysteine; 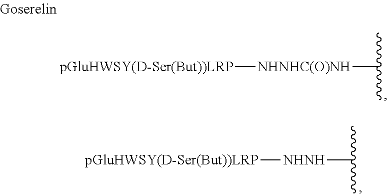
and its analogs as the structures shown below: 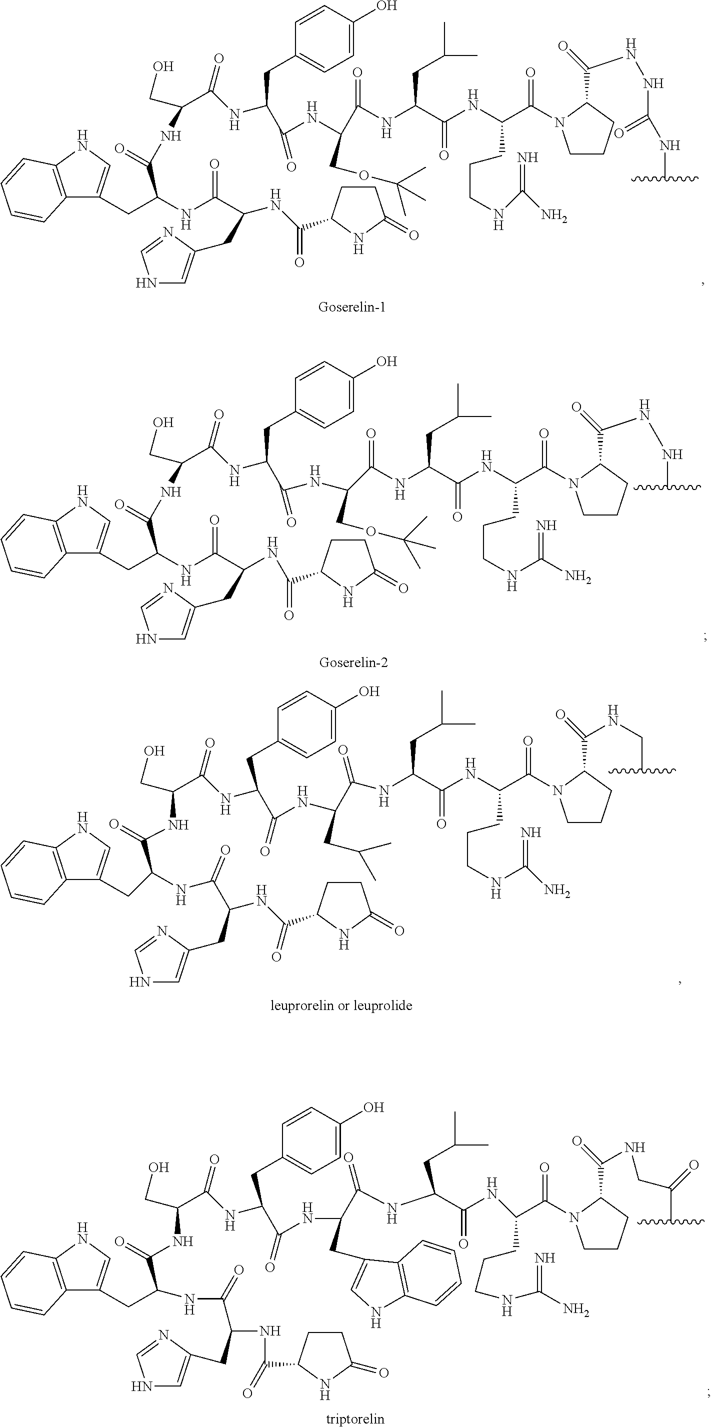 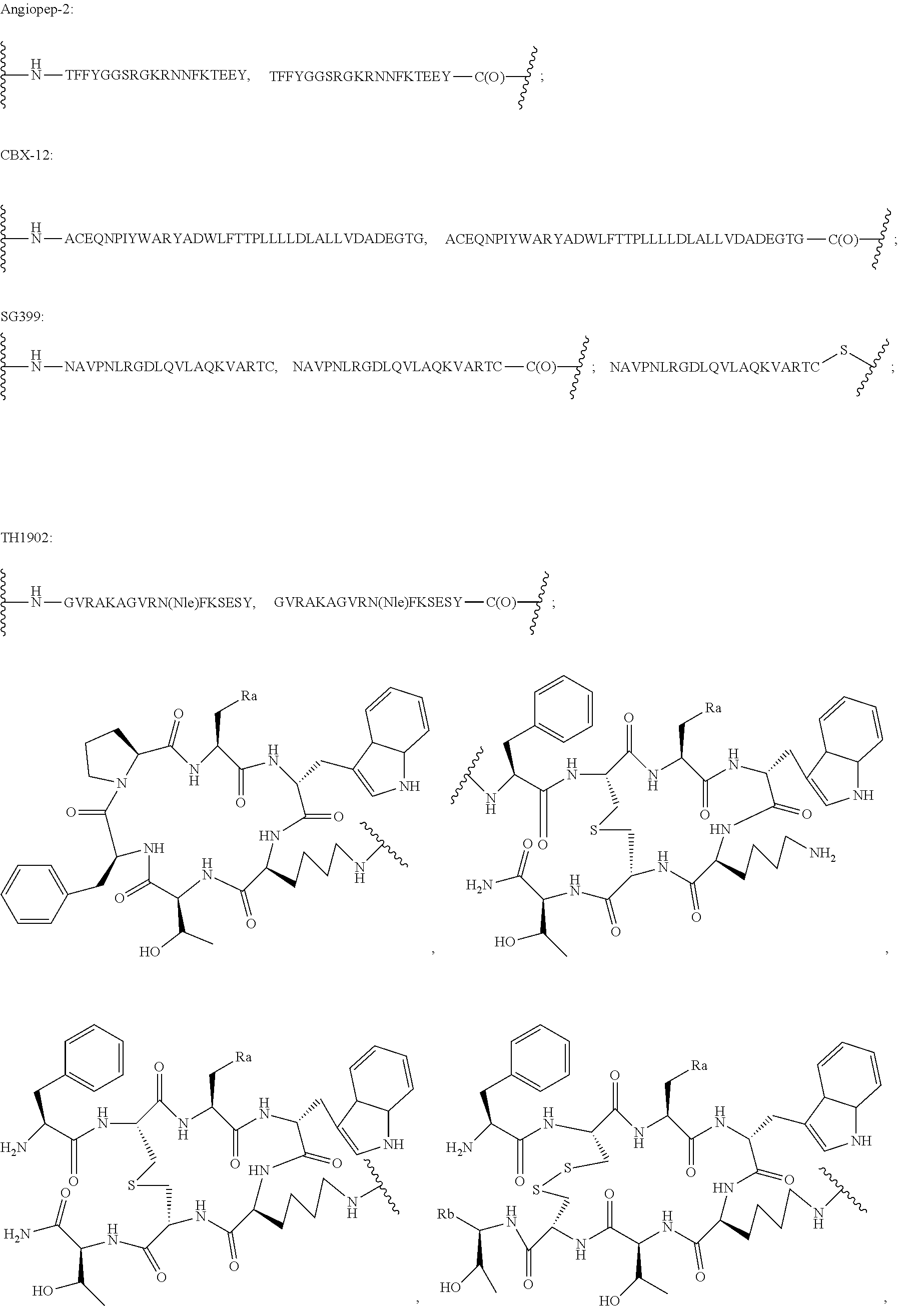 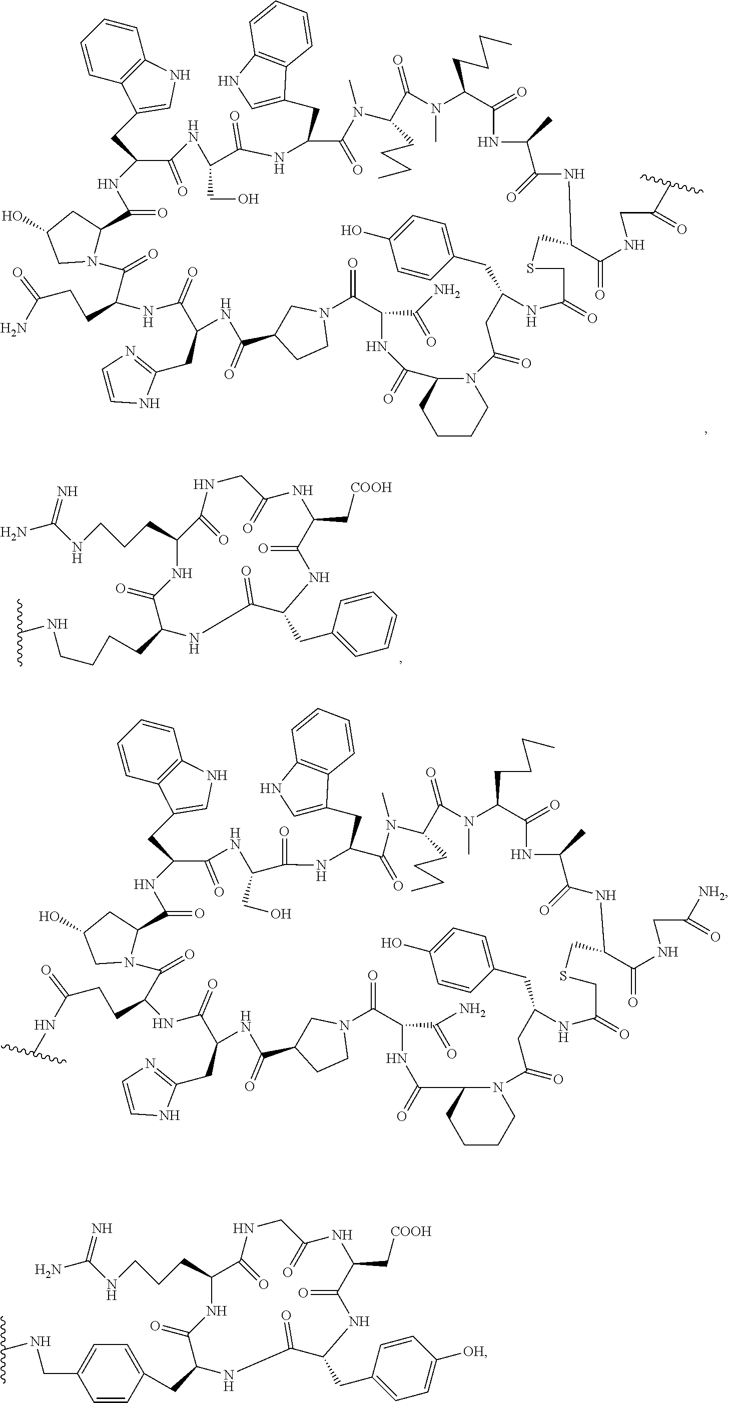 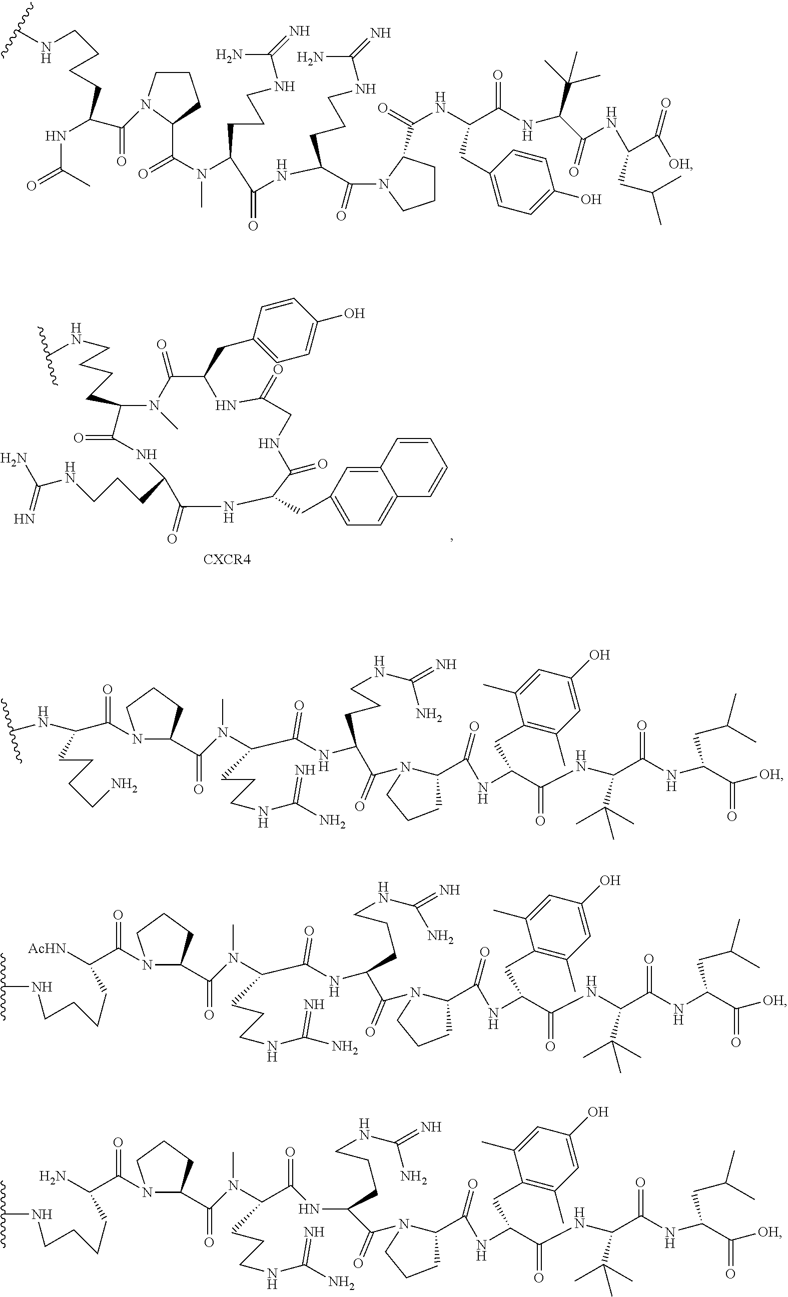 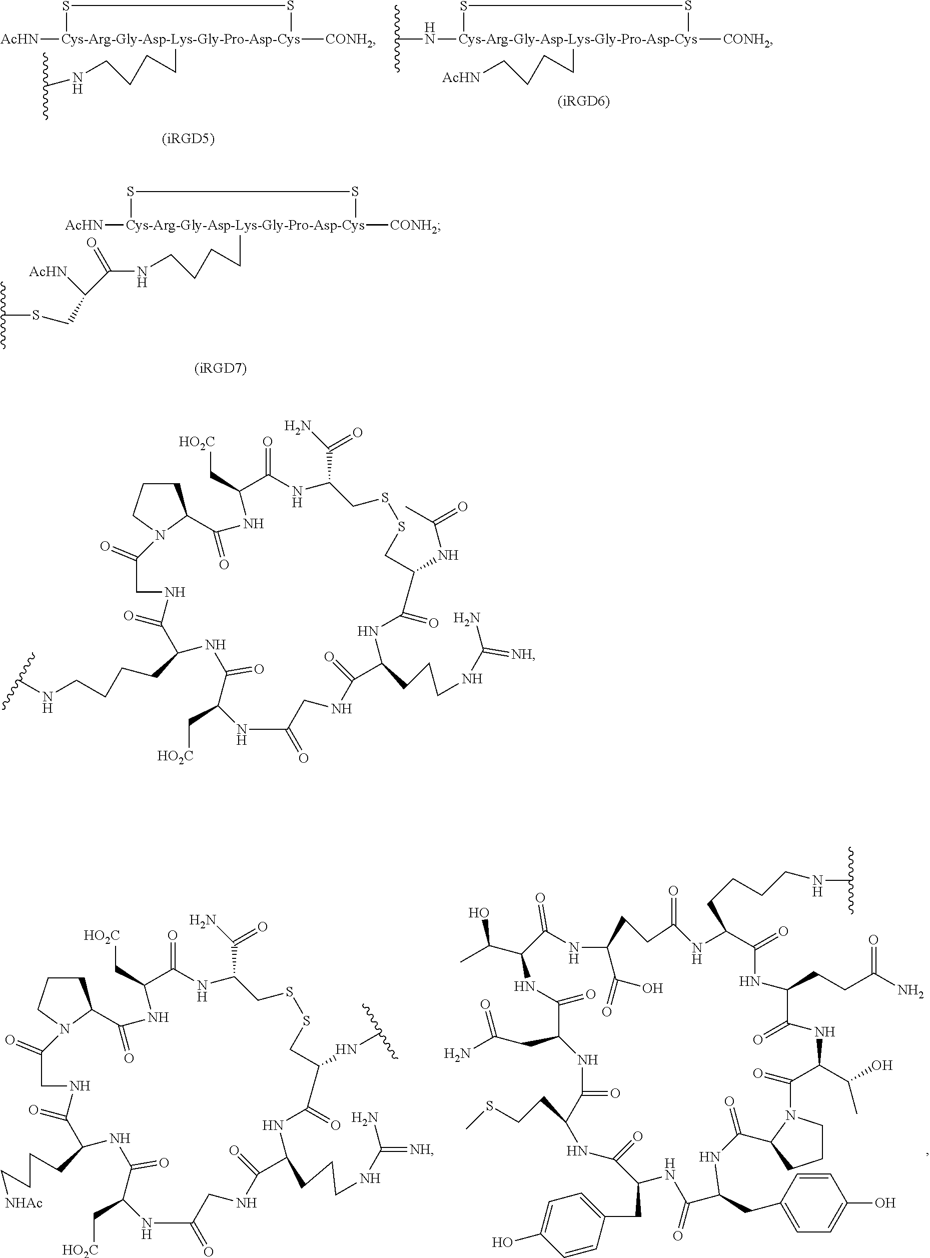 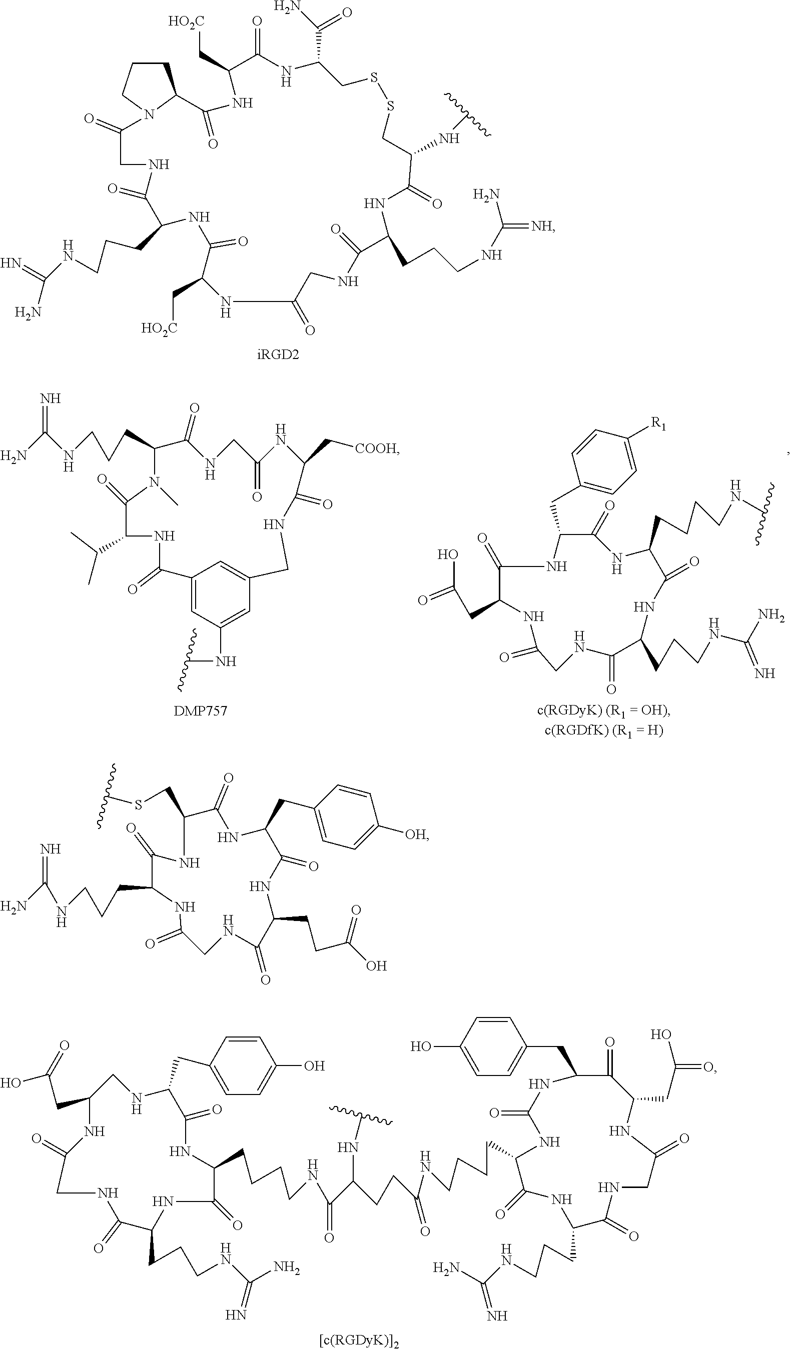 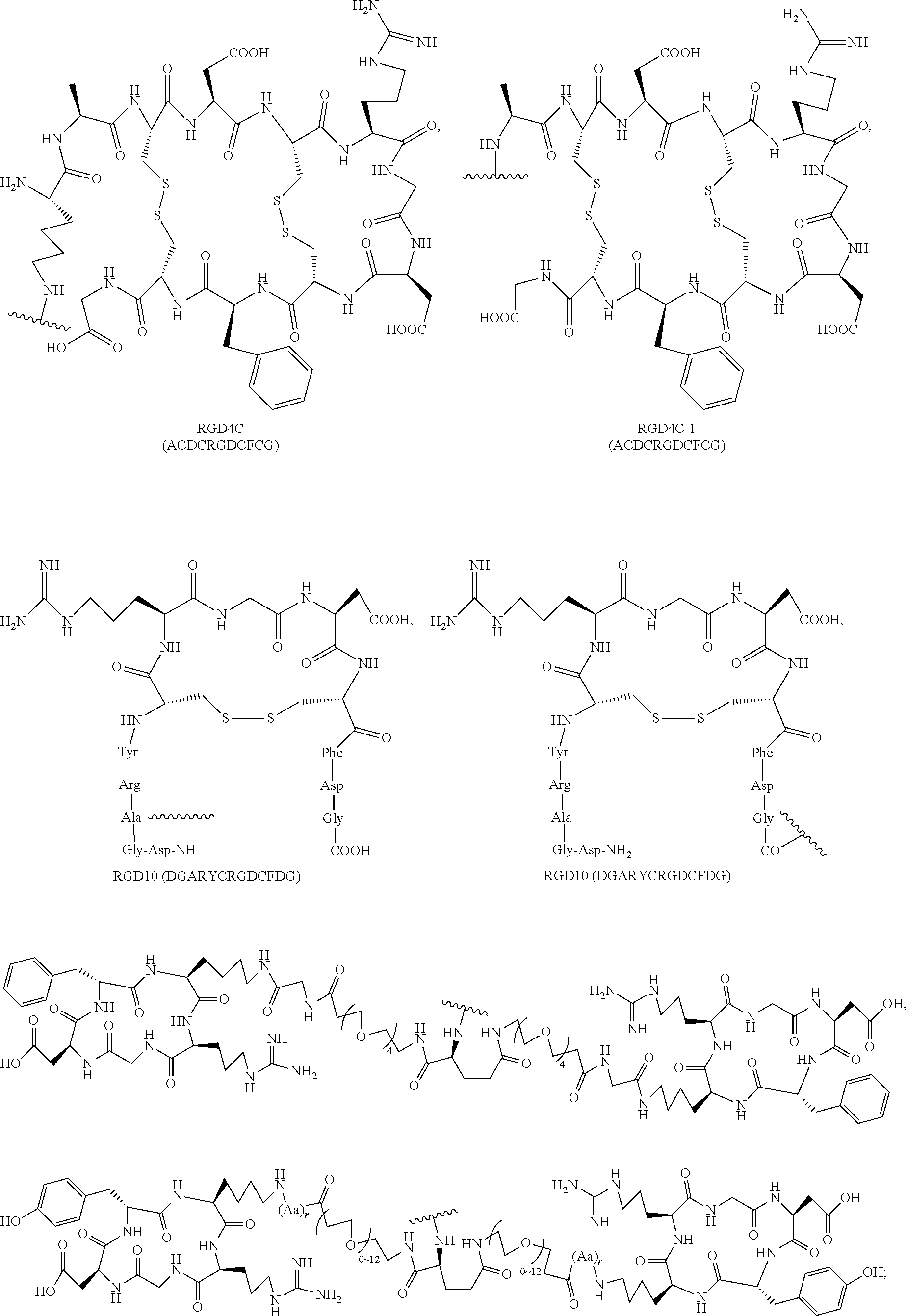 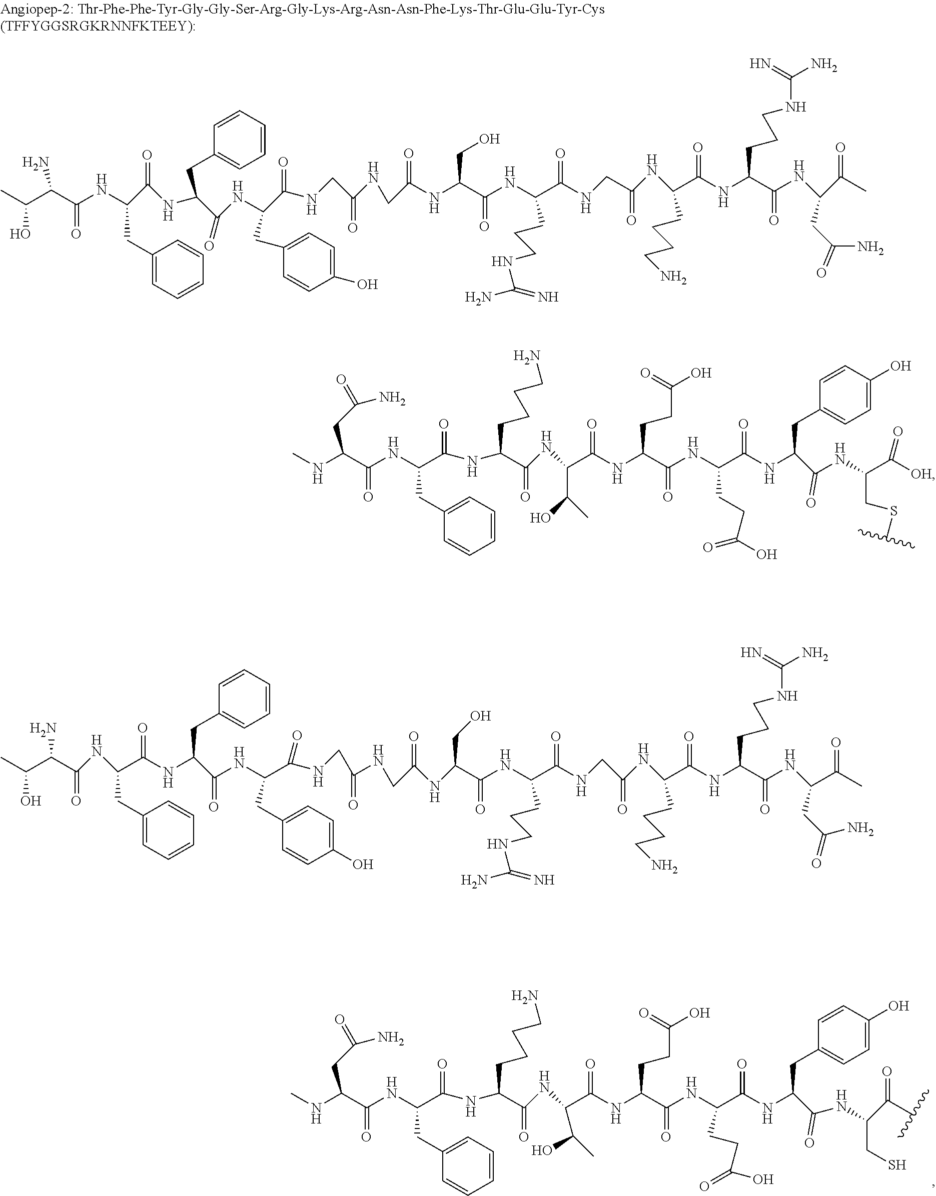 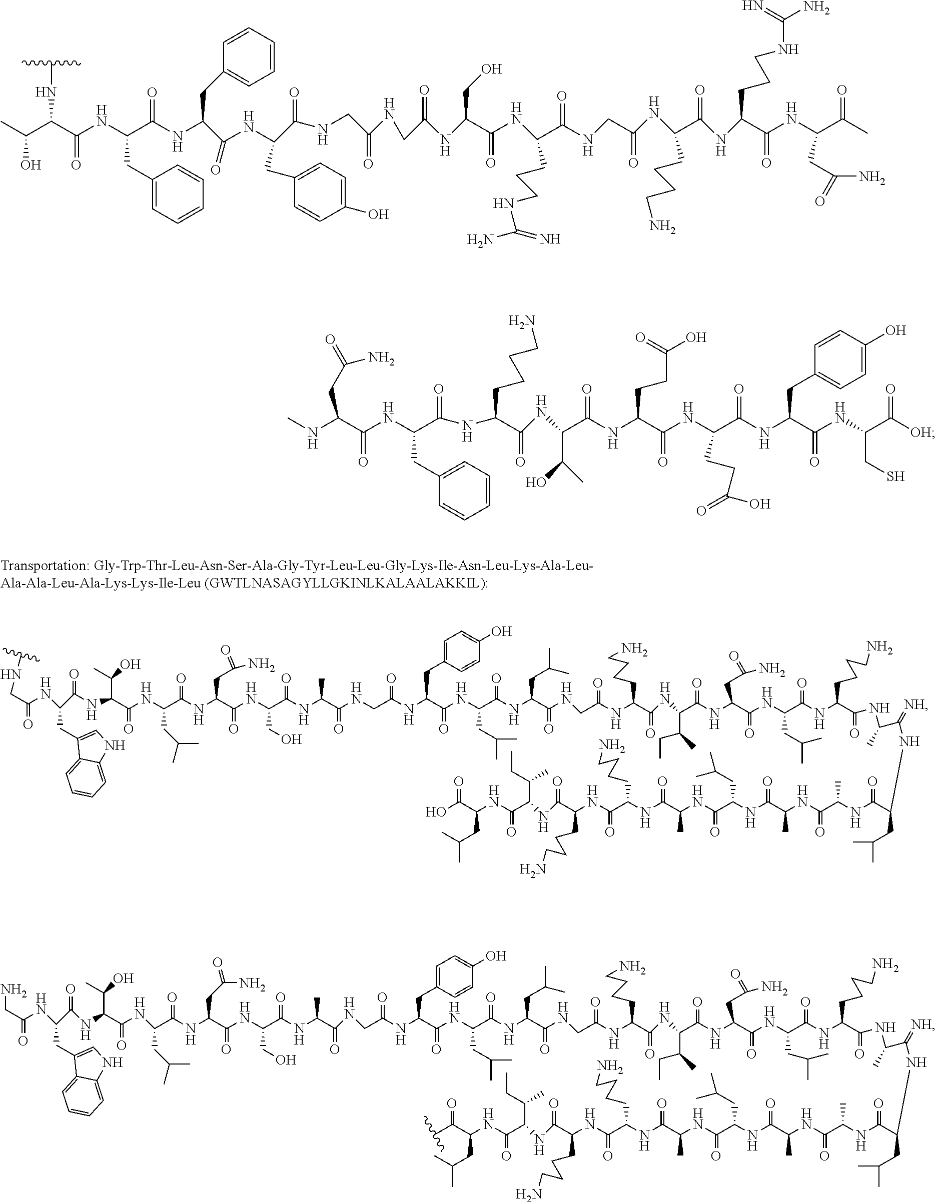 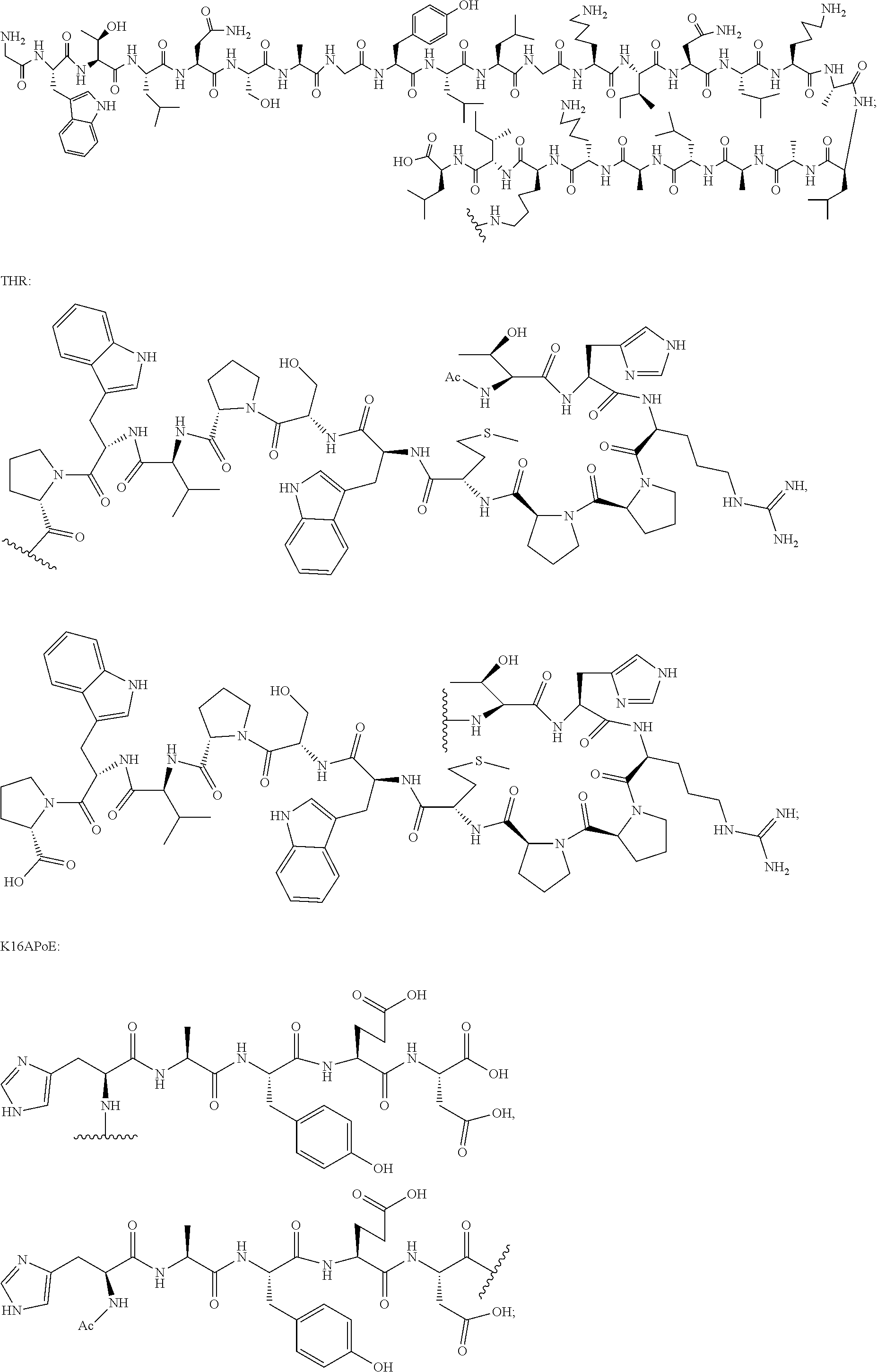 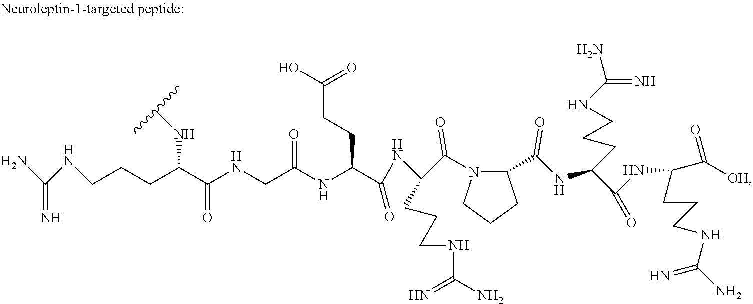 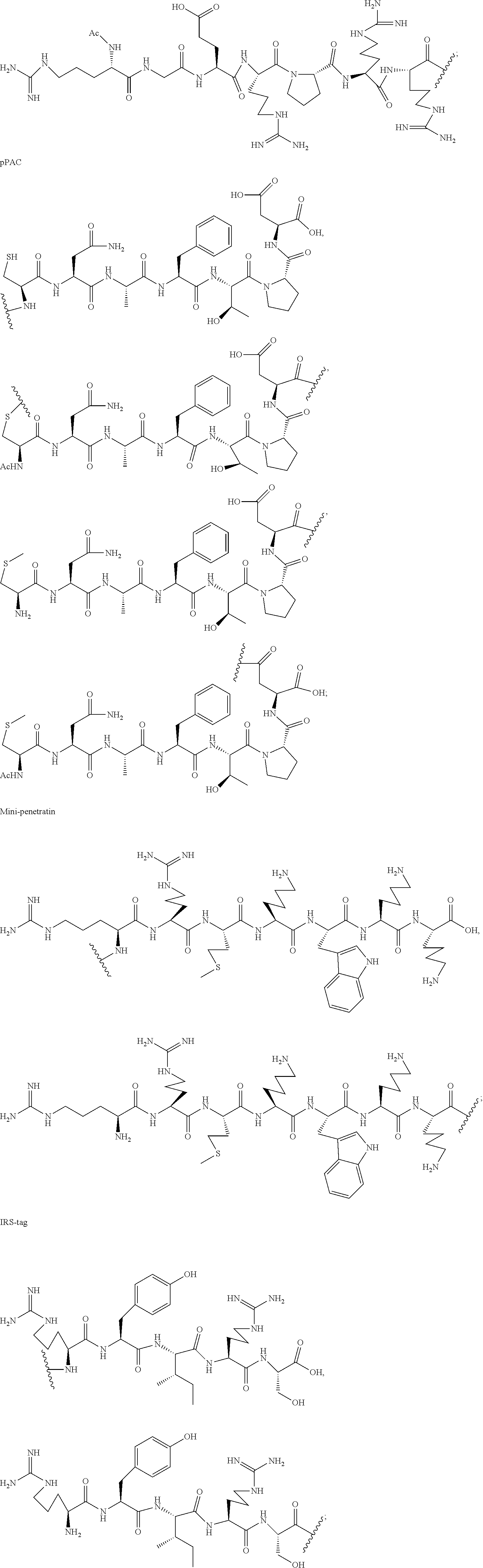 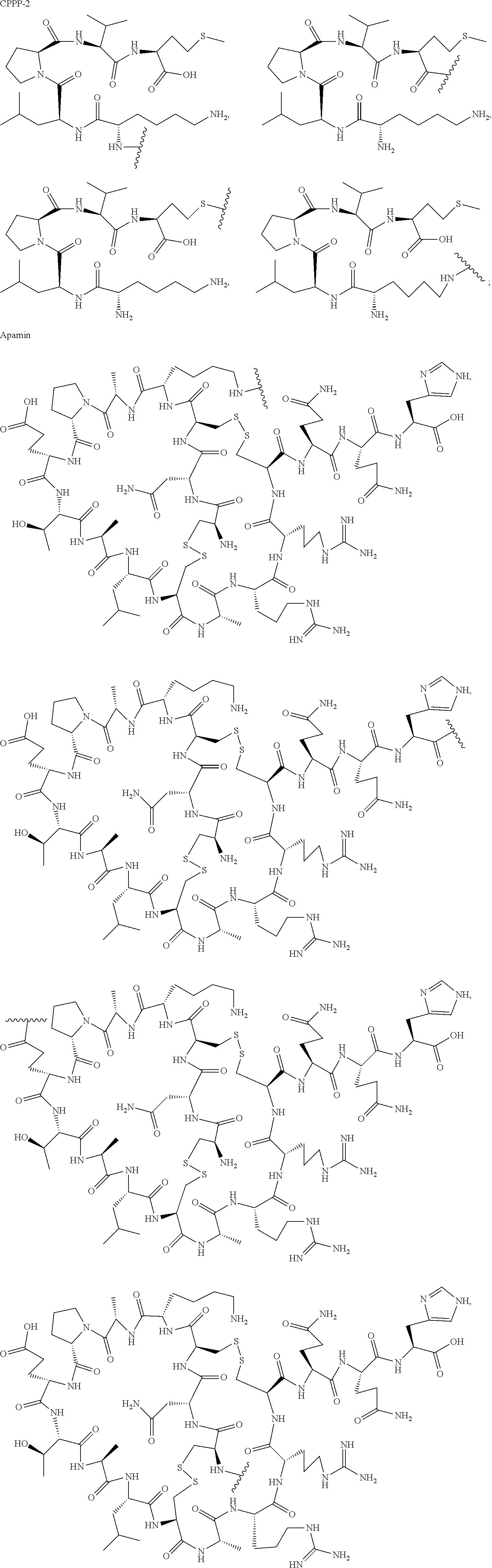 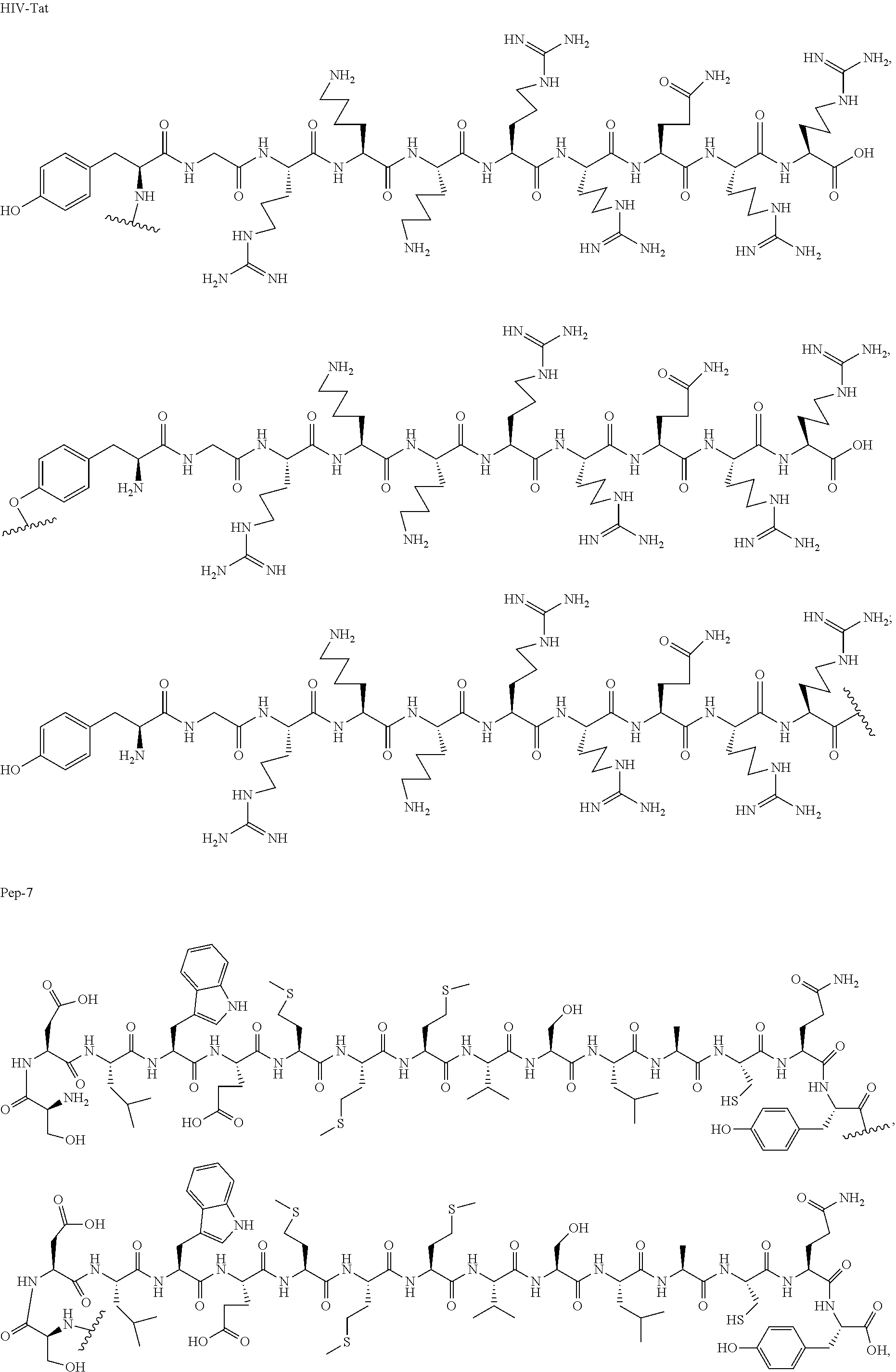 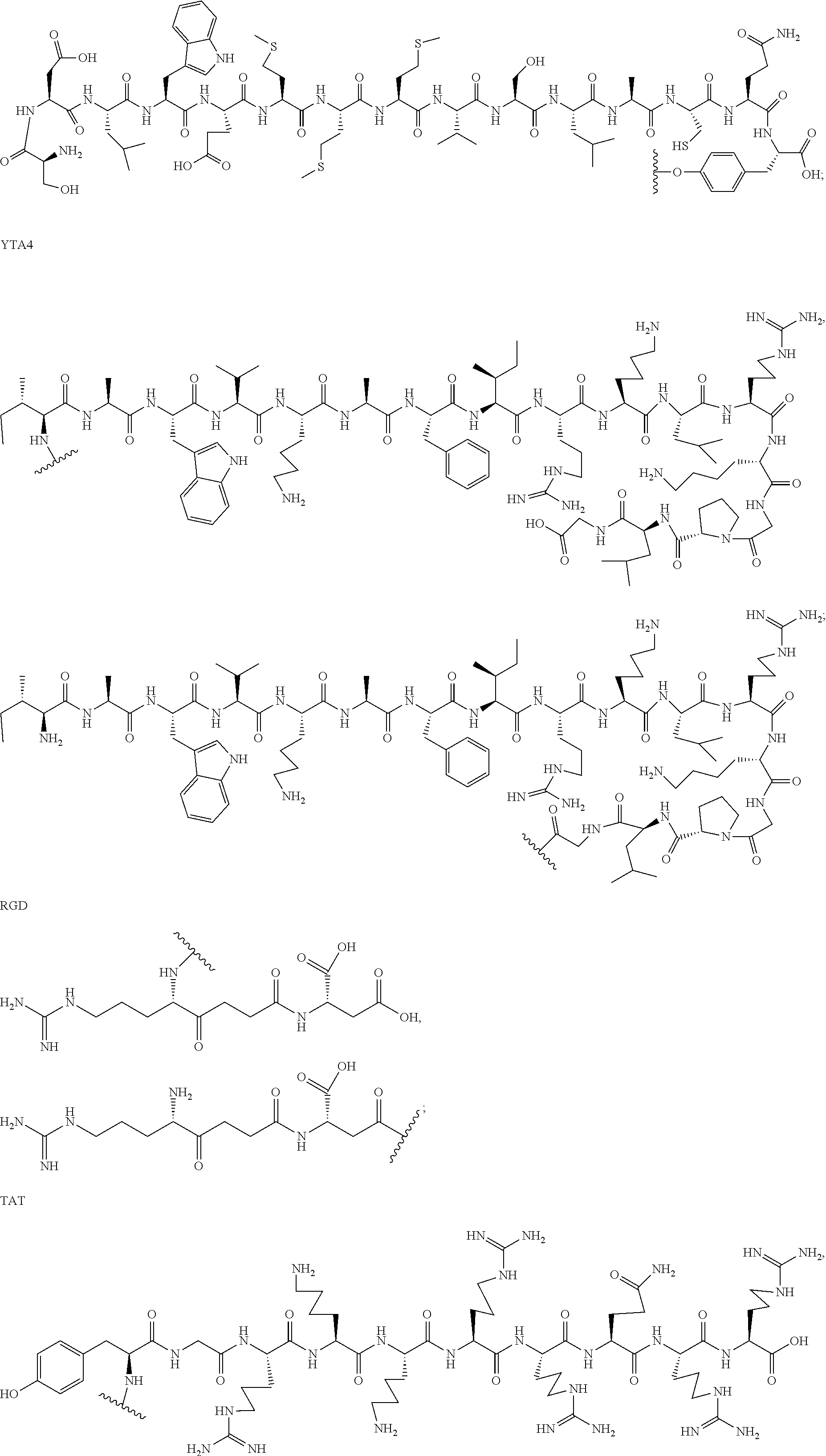 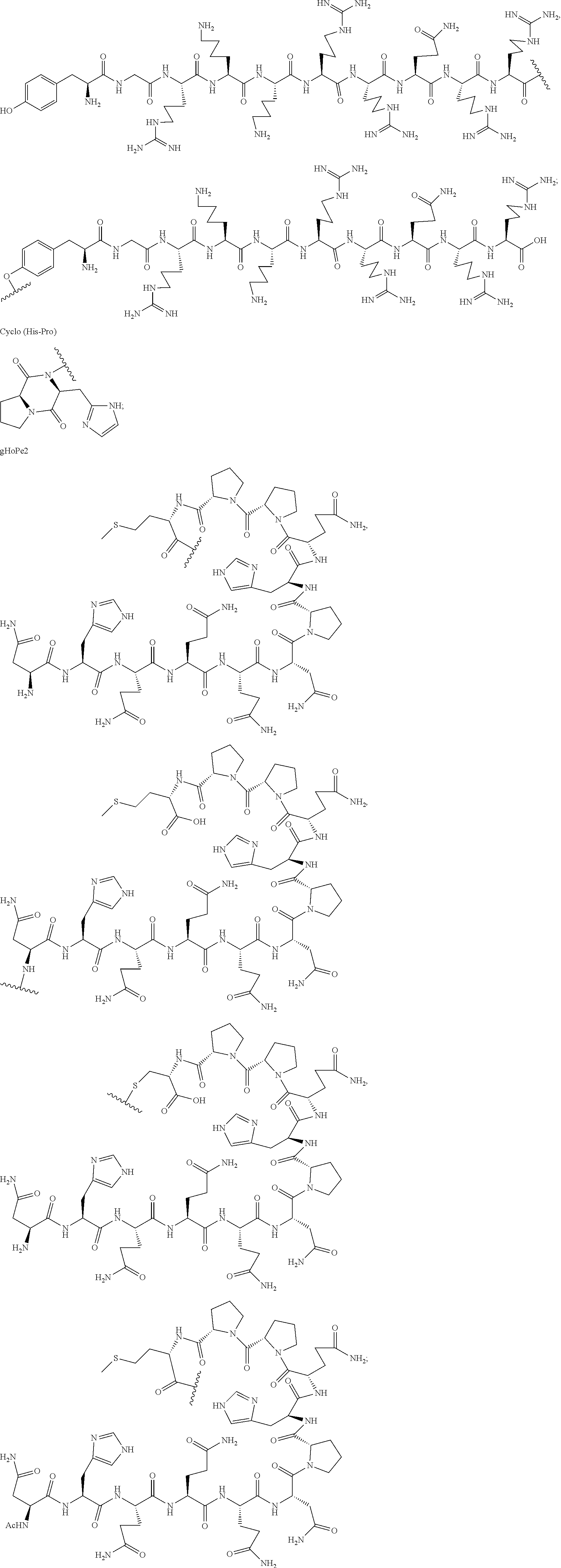 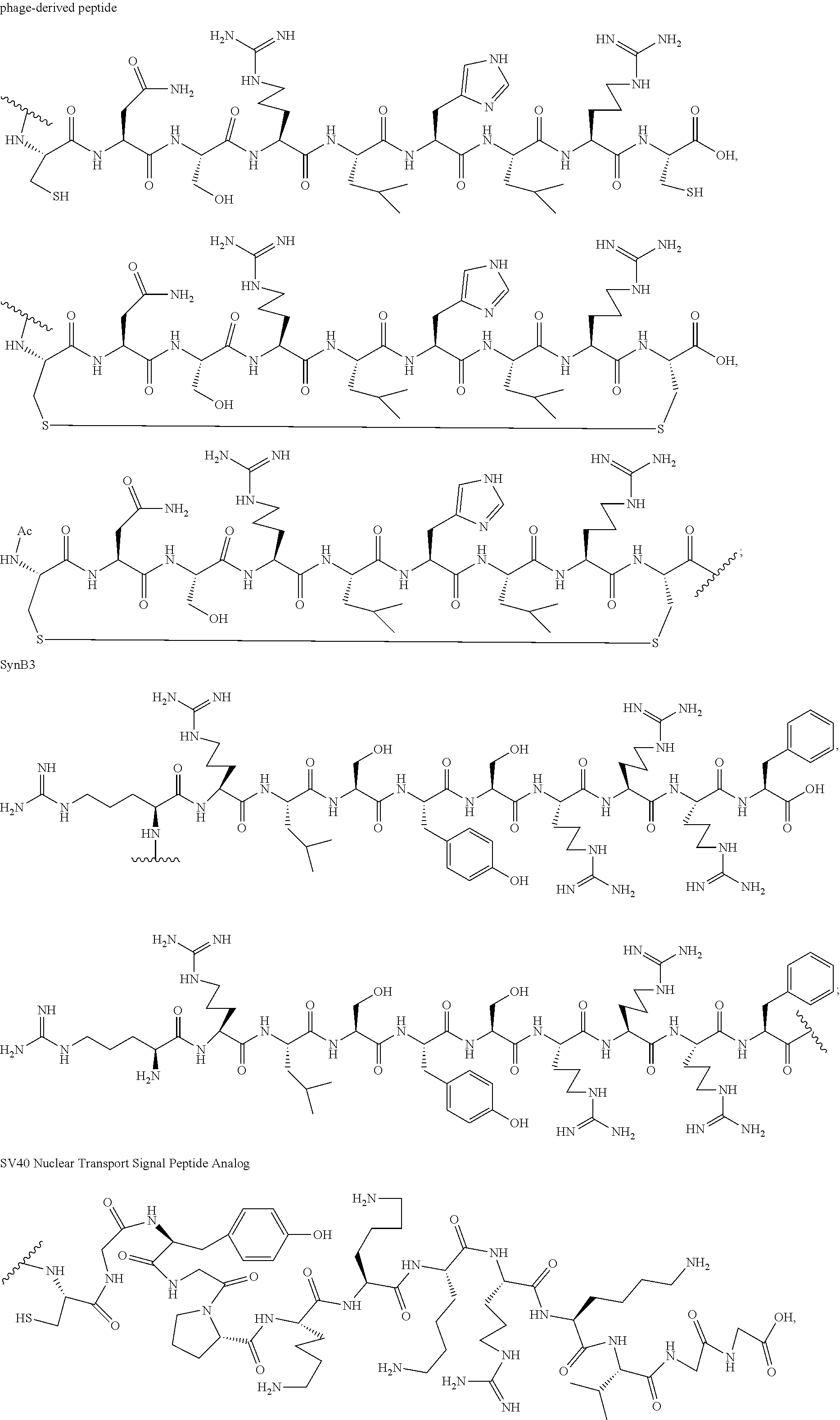 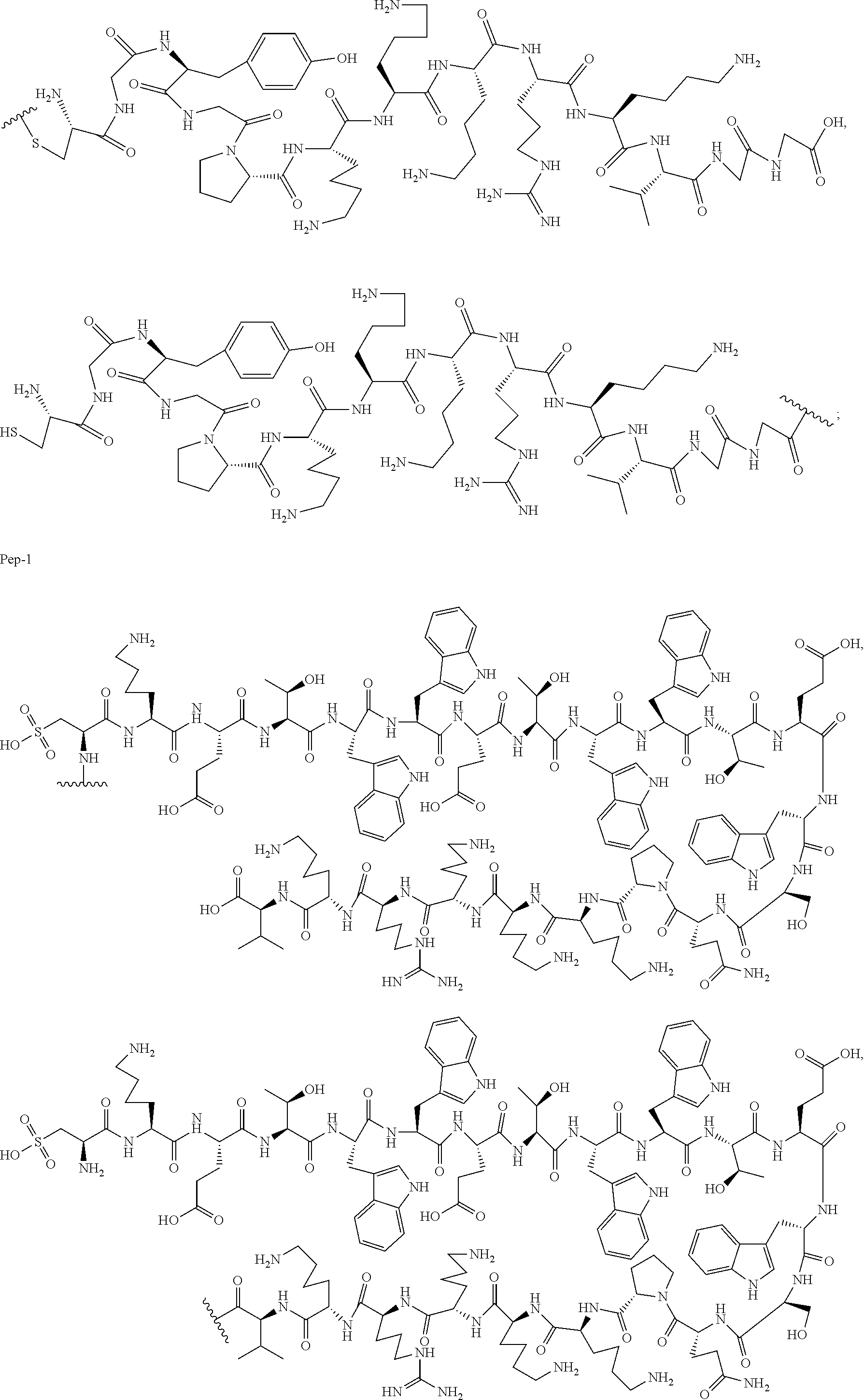 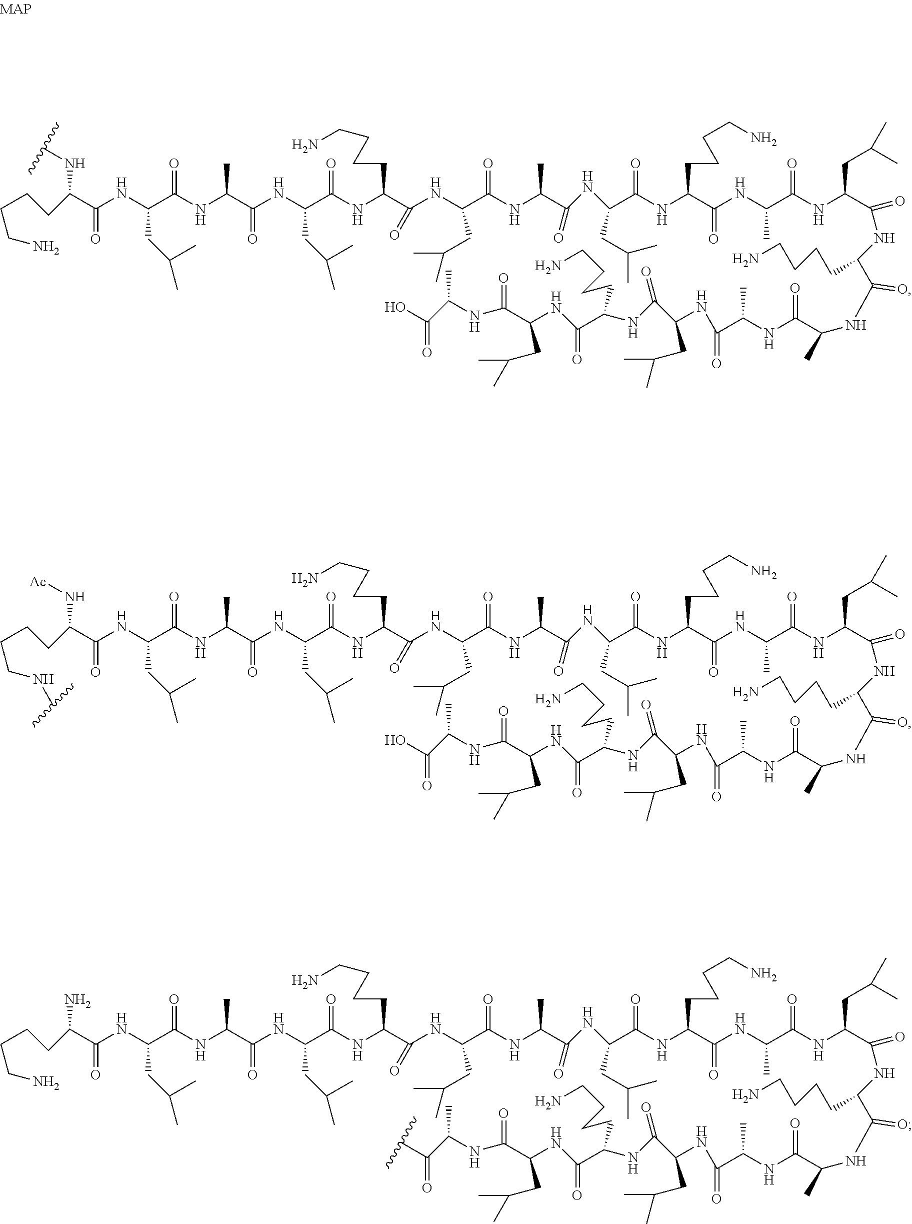 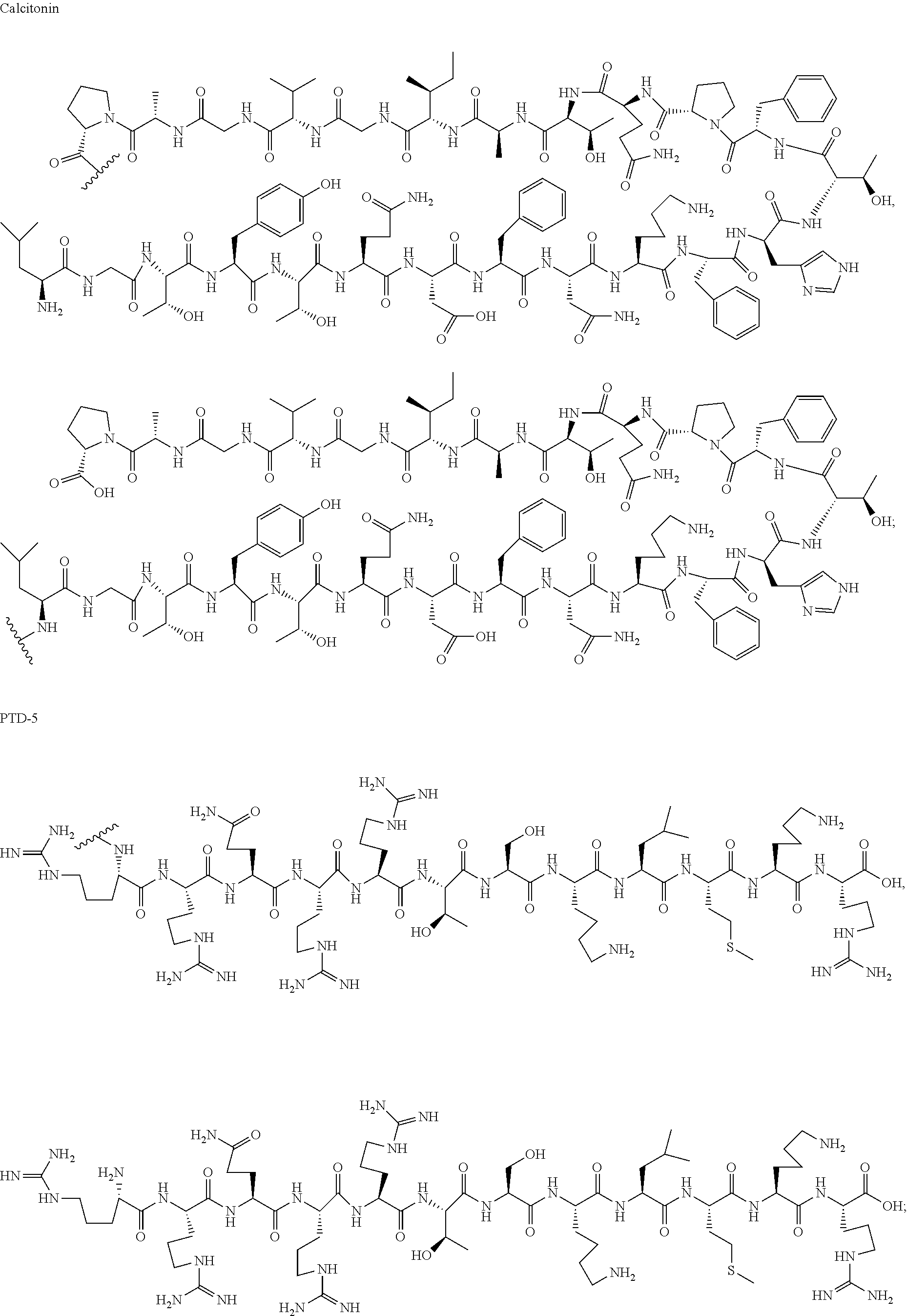 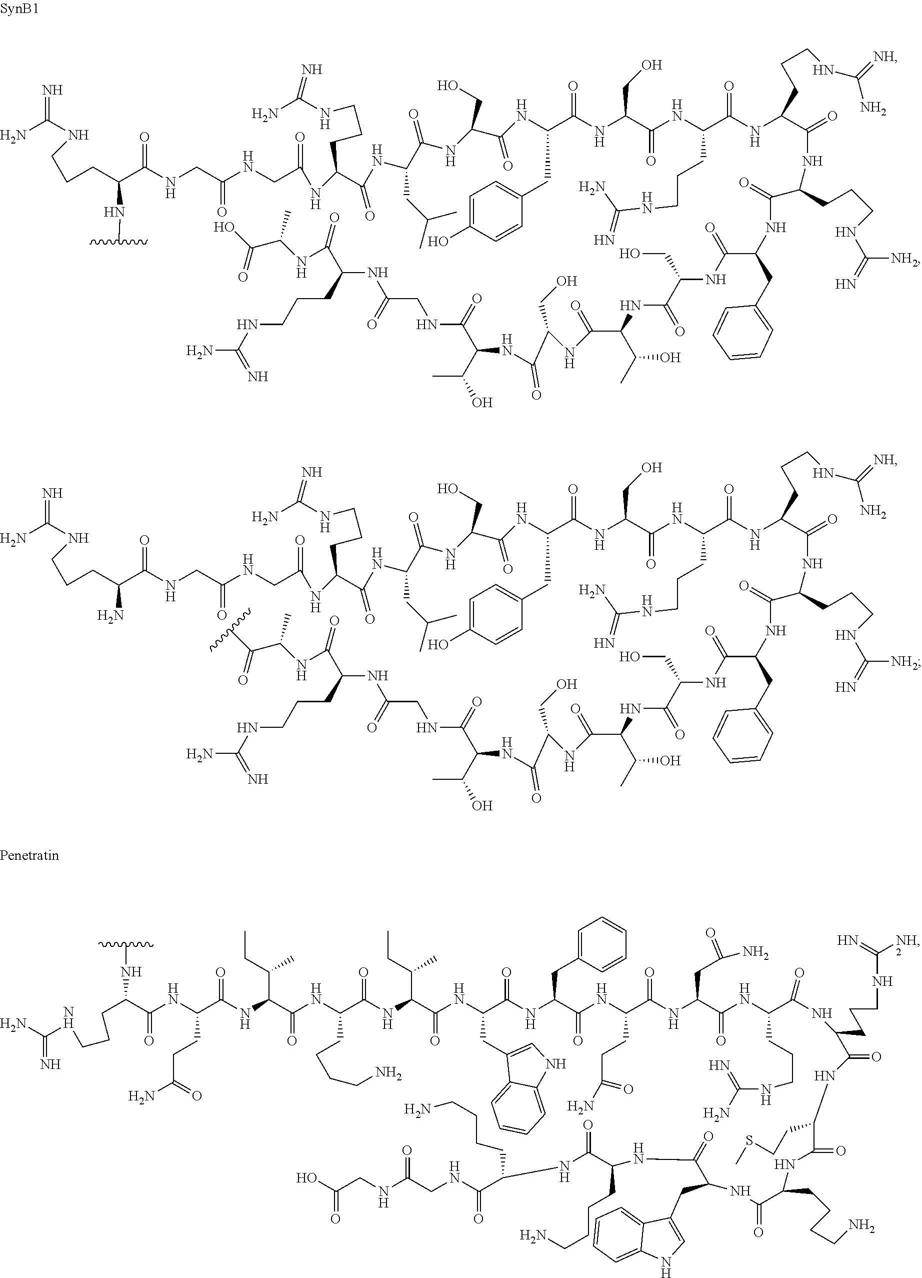 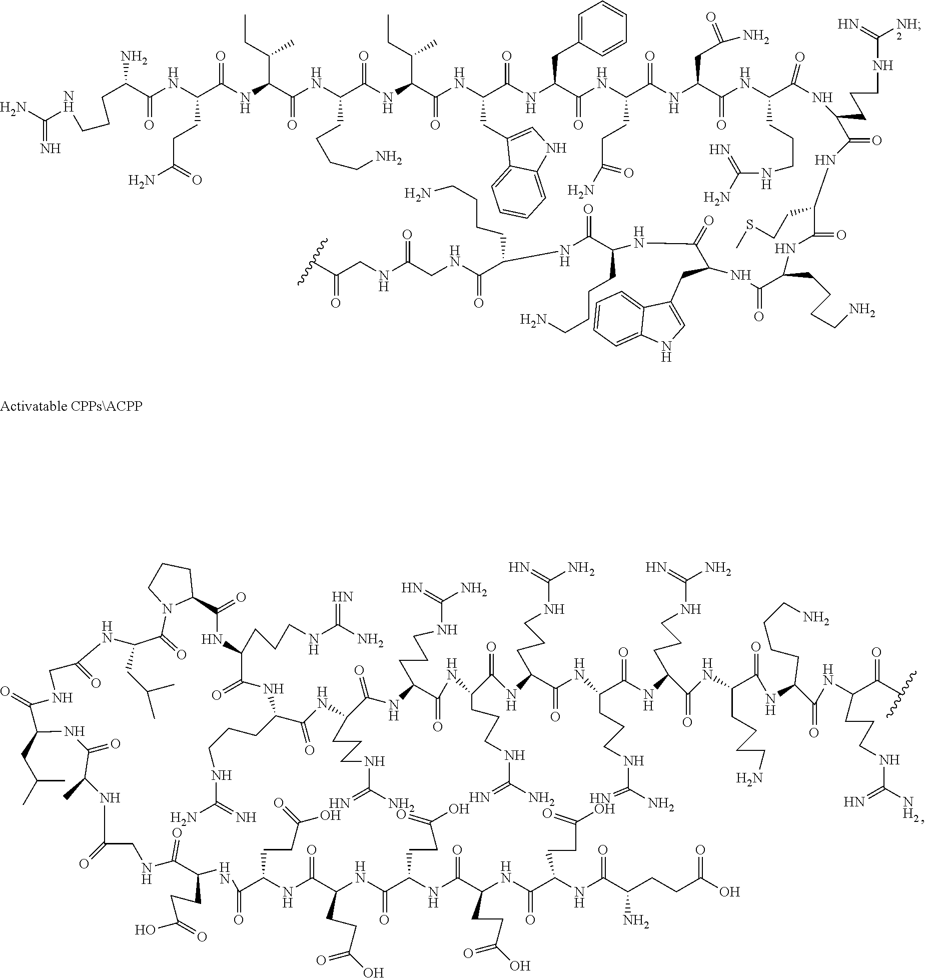 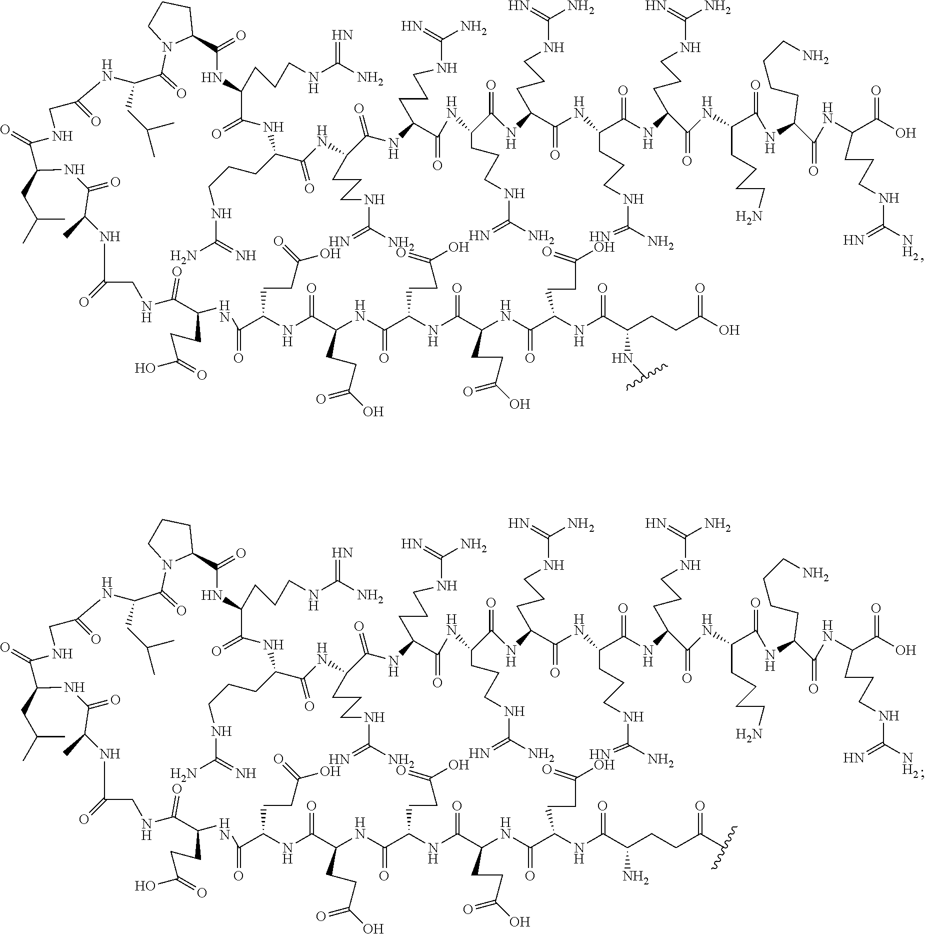 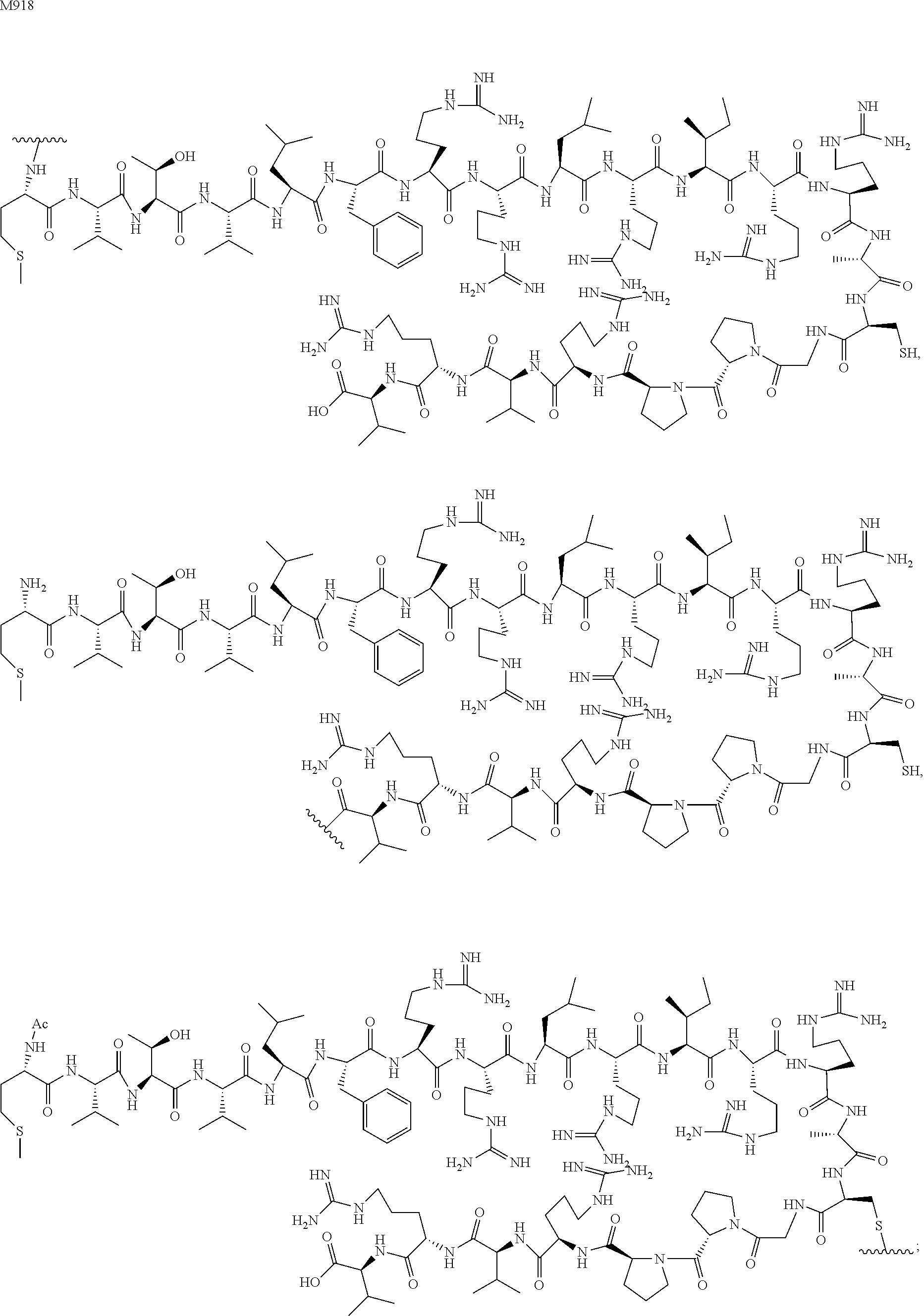 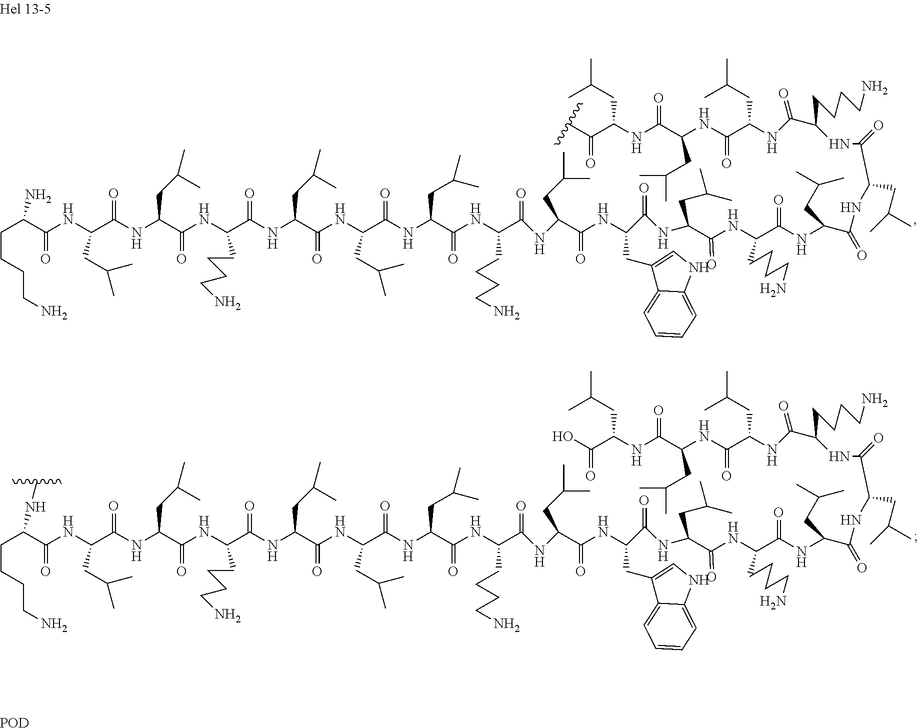 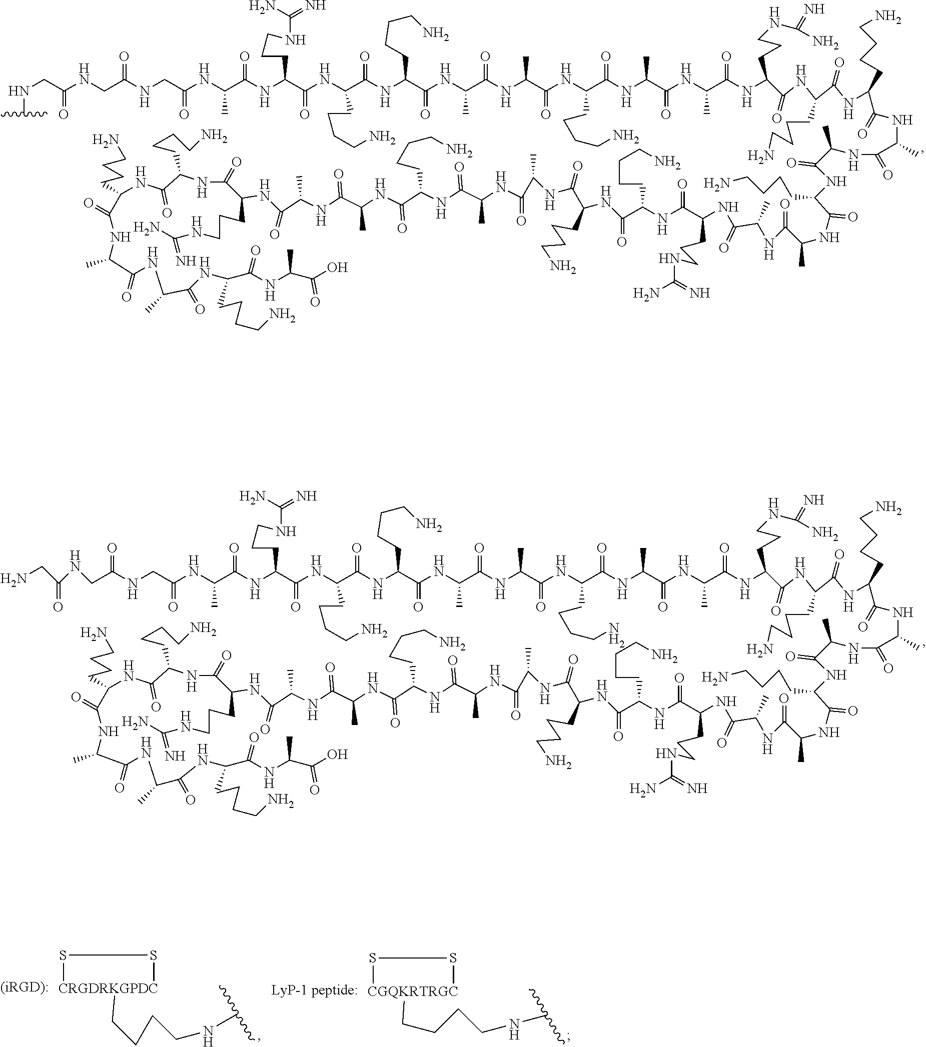
wherein [symbol] is the site linked to Ld₁, Ld₂, Ld₃, Ld₄, Ld₅, or Ld₆; Ra is Ar; Rb is OH, COOH, COOCH₃, CH₃OH, CH₃NH₂, or CONH₂.

3. The antibody drug conjugate according to claim 1, wherein the functional small molecule, A₁, A₂, A₃, A₄, A₅ and A₆ are independently a nucleoside antimetabolite, which is selected from:

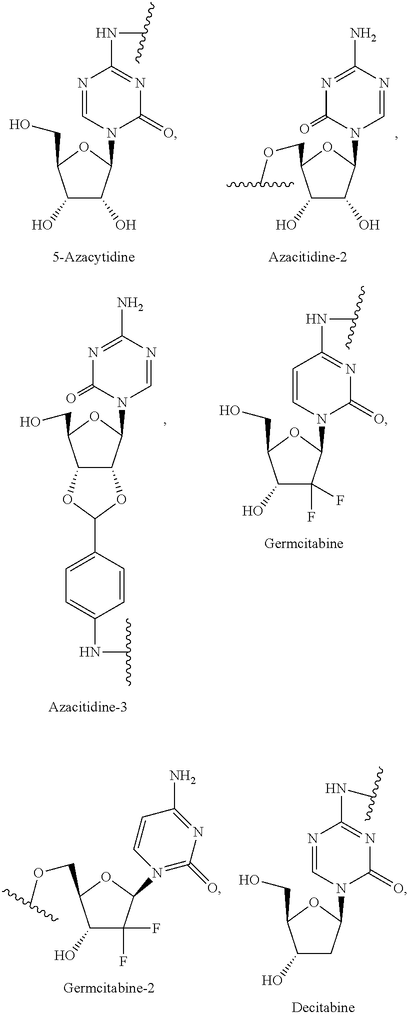

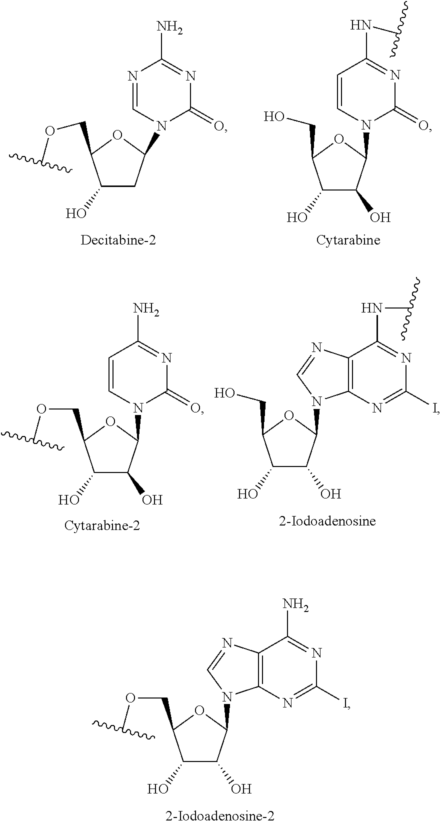

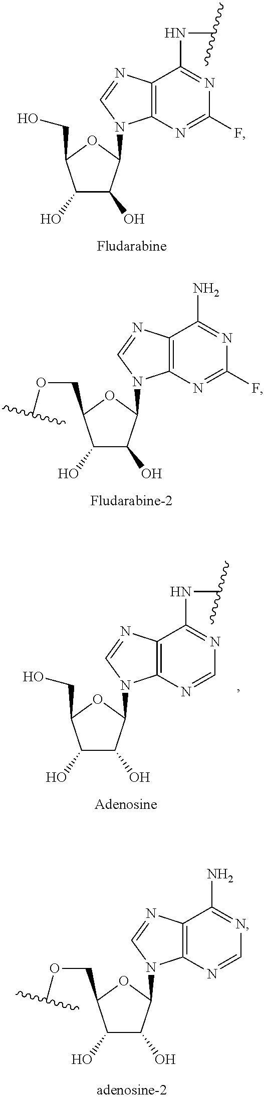 [SMILES: *Nc1nc(F)nc2c1ncn2[C@@H]1O[C@H](CO)[C@@H](O)[C@@H]1O ; *OC[C@H]1O[C@@H](n2cnc3c(N)nc(F)nc32)[C@@H](O)[C@@H]1O ; *Nc1ncnc2c1ncn2[C@@H]1O[C@H](CO)[C@@H](O)[C@H]1O ; *OC[C@H]1O[C@@H](n2cnc3c(N)ncnc32)[C@H](O)[C@@H]1O]

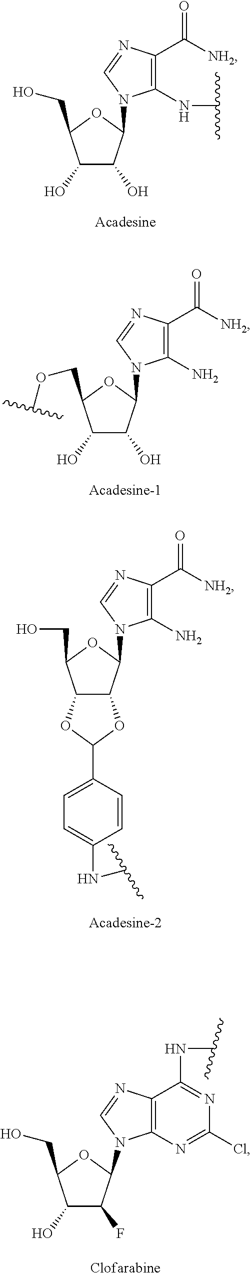

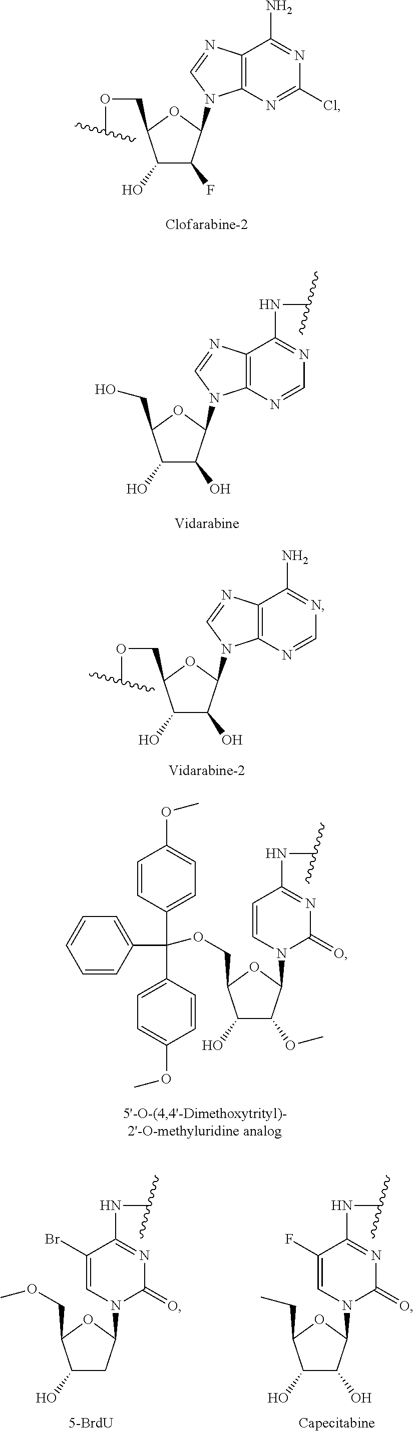 [SMILES: *OC[C@H]1O[C@@H](n2cnc3c(N)nc(Cl)nc32)[C@@H](F)[C@@H]1O ; *Nc1ncnc2c1ncn2[C@@H]1O[C@H](CO)[C@@H](O)[C@@H]1O ; *OC[C@H]1O[C@@H](n2cnc3c(N)ncnc32)[C@@H](O)[C@@H]1O ; *Nc1ccn([C@@H]2O[C@H](COC(c3ccccc3)(c3ccc(OC)cc3)c3ccc(OC)cc3)[C@@H](O)[C@H]2OC)c(=O)n1 ; *Nc1nc(=O)n([C@H]2C[C@H](O)[C@@H](COC)O2)cc1Br ; *Nc1nc(=O)n([C@@H]2O[C@H](CC)[C@@H](O)[C@H]2O)cc1F]

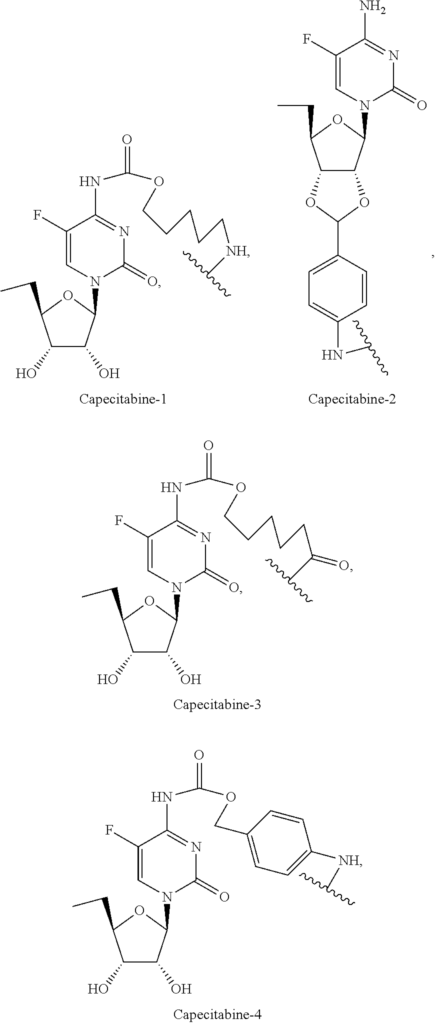

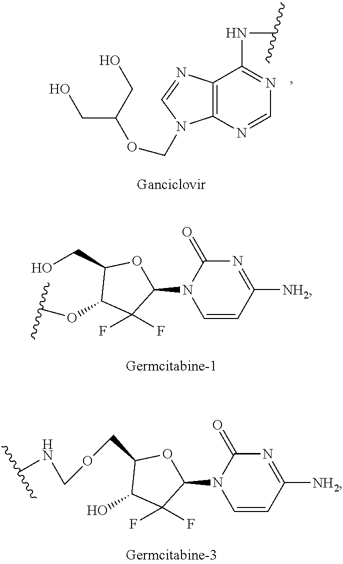 [SMILES: *Nc1ncnc2c1ncn2COC(CO)CO ; *O[C@@H]1[C@@H](CO)O[C@@H](n2ccc(N)nc2=O)C1(F)F ; *NCOC[C@H]1O[C@@H](n2ccc(N)nc2=O)C(F)(F)[C@@H]1O]

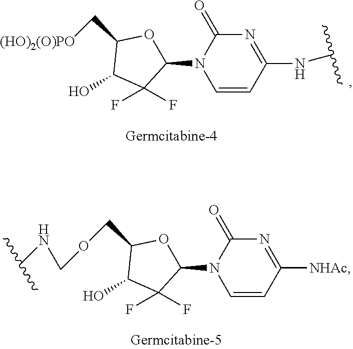

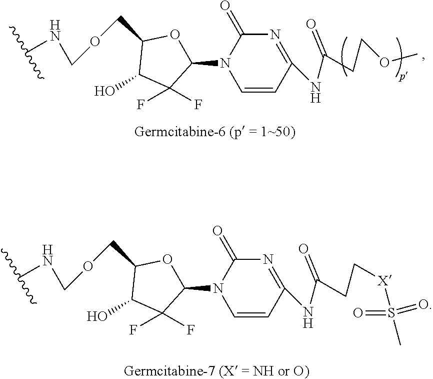

4. The antibody drug conjugate according to claim 1, wherein the cytotoxic drug, D₁ and D2, are independently selected from: tubulysin analog having the following formula (IV′): 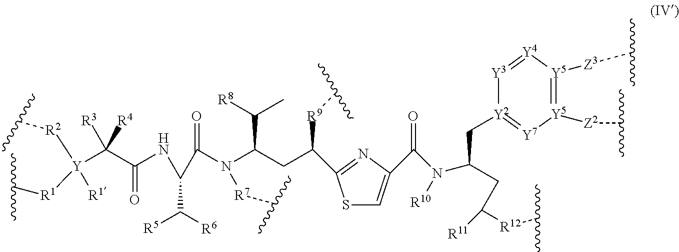
or a pharmaceutically acceptable salt, hydrates, or hydrated salt; or a polymorphic crystalline structure; or an optical isomer, racemate, diastereomer or enantiomer thereof,
wherein [symbol] is a linkage site that either one or two of them can link to L₁ and/or L₂ independently;
wherein R¹, R^{1′}, R², R³, and R⁴ are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or R¹R², R¹R³, R²R³, R³R⁴, or R⁵R⁶ form a 3˜7 membered carbocyclic, cycloalkyl, heterocyclic, heterocycloalkyl, aromatic or heteroaromatic ring system; R¹ and R² can be independently absent when they link to L₁ or L₂ independently or simultaneously, Y¹ is N or CH;
wherein R⁵, R⁶, R⁸, R¹⁰ and R¹¹ are independently H, or C₁˜C₄ alkyl or heteroalkyl;
wherein R⁷ is independently H, R¹⁴, —R¹⁴C(═O)X¹R¹⁵; or —R¹⁴X¹R¹⁵, X₁ is O, S, S—S, NH, CH₂ or NR¹⁴,
wherein R⁹ is selected from H, OH, ═O, —OR¹⁴, —OC(═O)R¹⁴, —OC(—O)NHR¹⁴, or —OC(═O) NR¹⁴R¹⁵; when R⁹ links L₁ or L₂, R⁹ is —O—, —OC(═O)NH— or —OC(═O)N(R¹⁴)—;
wherein R¹¹ is H, or C₁˜C₈ alkyl or C₃˜C₈ Ar-alkyl;
wherein R¹² is —COOH, —COSH, —CONH₂, CONHNH₂, CONHNHR¹⁵, —CONH(R¹⁵), —COOR¹⁵, —R¹⁵COR¹⁶, —R¹⁵COOR¹⁶, —R¹⁵C(O)NH₂, —R¹⁵C(O)NHR¹⁶, —COSR¹⁵, R¹⁵S(═O)₂R¹⁶, —R¹⁵P(═O)(OR¹⁷)₂, —R¹⁵OP(═O)(OR¹⁷)₂, —COOCH₂OP(═O)(OR¹⁷)₂, —COX₂SO₂R¹⁷, —COOR¹⁵X₂R¹⁶, tetrazole, imidazole, or triazole, where X₂ is —O—, —S—, —NH—, —N(R¹⁵)—, —O—R¹⁵—, —S—R¹⁵—, CH₂ or —NHR¹⁵—; when R¹² links L₁ or L₂, R¹² is —C(O)O—, —C(O)NH—, —C(═O)NHS(O)₂R¹⁵— or —C(═O)N(R¹⁵)—;
R¹³ and R¹⁴ are independently C₁˜C₈ alkyl, heteroalkyl; C₂-C₈ of alkenyl, alkynyl, heteroalkyl, heterocycloalkyl; C₃-C₈ of aryl, Ar-alkyl;
Z² and Z³ are independently H, O, S, NH, N(R¹⁵), NHNH, —OH, —SH, —NH₂, NHNH₂, —NH(R¹⁵), —OR¹⁵, CO, —COX², —COX²R¹⁶, R¹⁷, F, Cl, Br, I, SR¹⁶, NR¹⁶R¹⁷, N═NR¹⁶, N═R¹⁶, NO₂, SOR¹⁶R¹⁷, SO₂R¹⁶, SO₃R¹⁶, OSO₃R¹⁶, PR¹⁶R¹⁷, POR¹⁶R¹⁷, PO₂R¹⁶R¹⁷, OP(O)(OR¹⁷)₂, OCH₂OP(O)(OR¹⁷)₂, OC(O)R¹⁷, OC(O)OP(O)(OR¹⁷)₂, PO(OR¹⁶)(OR¹⁷), OP(O)(OR¹⁷)OP(O)(OR¹⁷)₂, OC(O)NHR¹⁷; —O—(C₄-C₁₂ glycoside), —N—(C₄-C₁₂ glycoside); C₁˜C₈ alkyl, heteroalkyl; C₂-C₈ of alkenyl, alkynyl, heteroalkyl, heterocycloalkyl; C₃-C₈ of aryl, Ar-alkyl, carbocyclic, cycloalkyl, heteroalkylcycloalkyl, alkylcarbonyl, heteroaryl, or 2-8 carbon atoms of esters, ether, or amide; or peptides containing 1-8 amino acids (NH(Aa)_{1˜8}, or CO(Aa)_{1˜8} (which are respectively N-terminal or C-terminal 1-8 the same or different amino acids)), or polyethyleneoxy unit of formula (OCH₂CH₂) p or (OCH₂CH(CH₃)) p, wherein p is an integer from 0 to about 100, or combination of above groups thereof; X₂ is O, S, S—S, NH, CH₂, OH, SH, NH₂, CHR¹⁵ or NR¹⁵;
R¹⁵, R¹⁶ and R¹⁷ are independently H, C₁˜C₈ alkyl, heteroalkyl; C₂-C₈ of alkenyl, alkynyl, heteroalkyl, heterocycloalkyl; C₃-C₈ of aryl, Ar-alkyl, cycloalkyl, heteroalkylcycloalkyl, alkylcarbonyl, heteroaryl, alkylcarbonyl, or Na⁺, K⁺, Cs⁺, Li⁺, Ca²⁺, Mg⁺, Zn²⁺, N⁺(R¹)(R²)(R³)(R⁴), HN⁺ (C₂H₅OH)₃ salt;
Y¹ and Y² are independently N or CH; q is 0 or 1; when q=0, Y³ does not exist, Y⁴, Y⁵, Y⁶ and Y⁷ are independently CH, N, NH, O, S, or N(R¹), thus Y², Y⁴, Y⁵, Y⁶ and Y⁷ form a heteroaromatic ring of furan, pyrrole thiophene, thiazole, oxazole and imidazole, pyrazole, triazole, tetrazole, thiadiazole; when q=1, Y³, Y⁴, Y⁵, Y⁶ and Y⁷ are independently CH or N, thus Y², Y³, Y⁴, Y⁵, Y⁶ and Y⁷ form aromatic ring of benzene, pyridine, pyridazine, pyrimidine, pyrazine, triazine, tetrazine, pentazine.

5. The antibody drug conjugate according to claim 1, wherein the cytotoxic drug, D₁ and D₂, are independently selected from: tubulysin analog having the following formula: 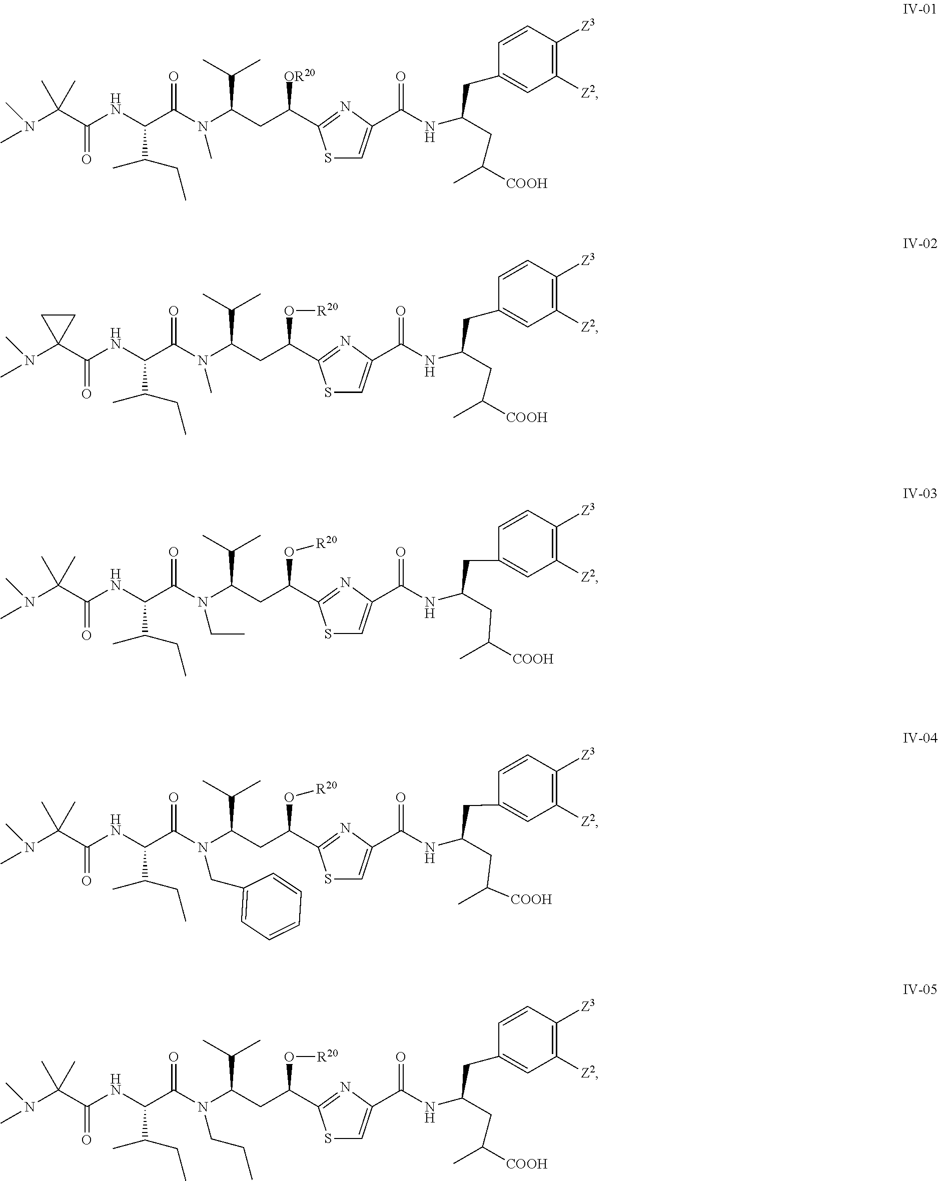 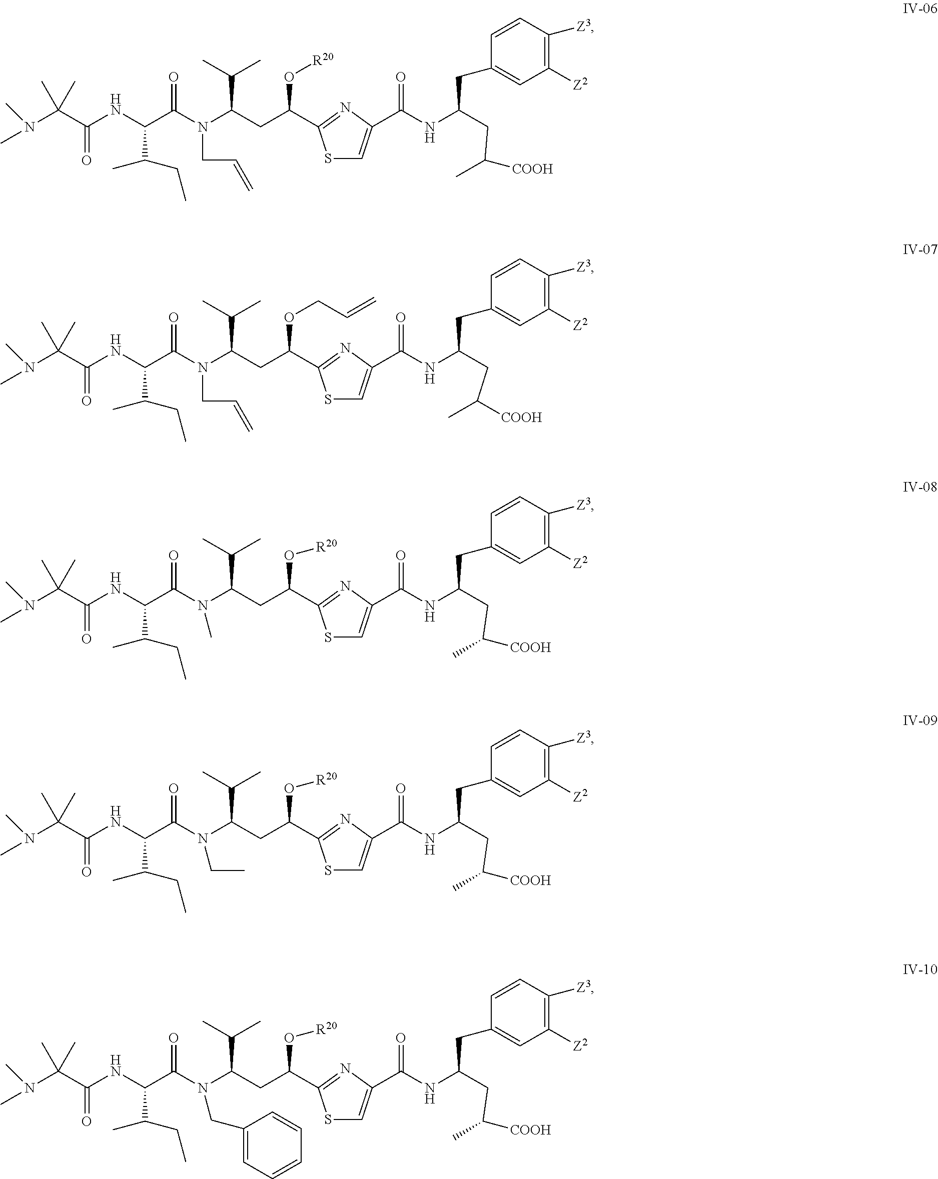 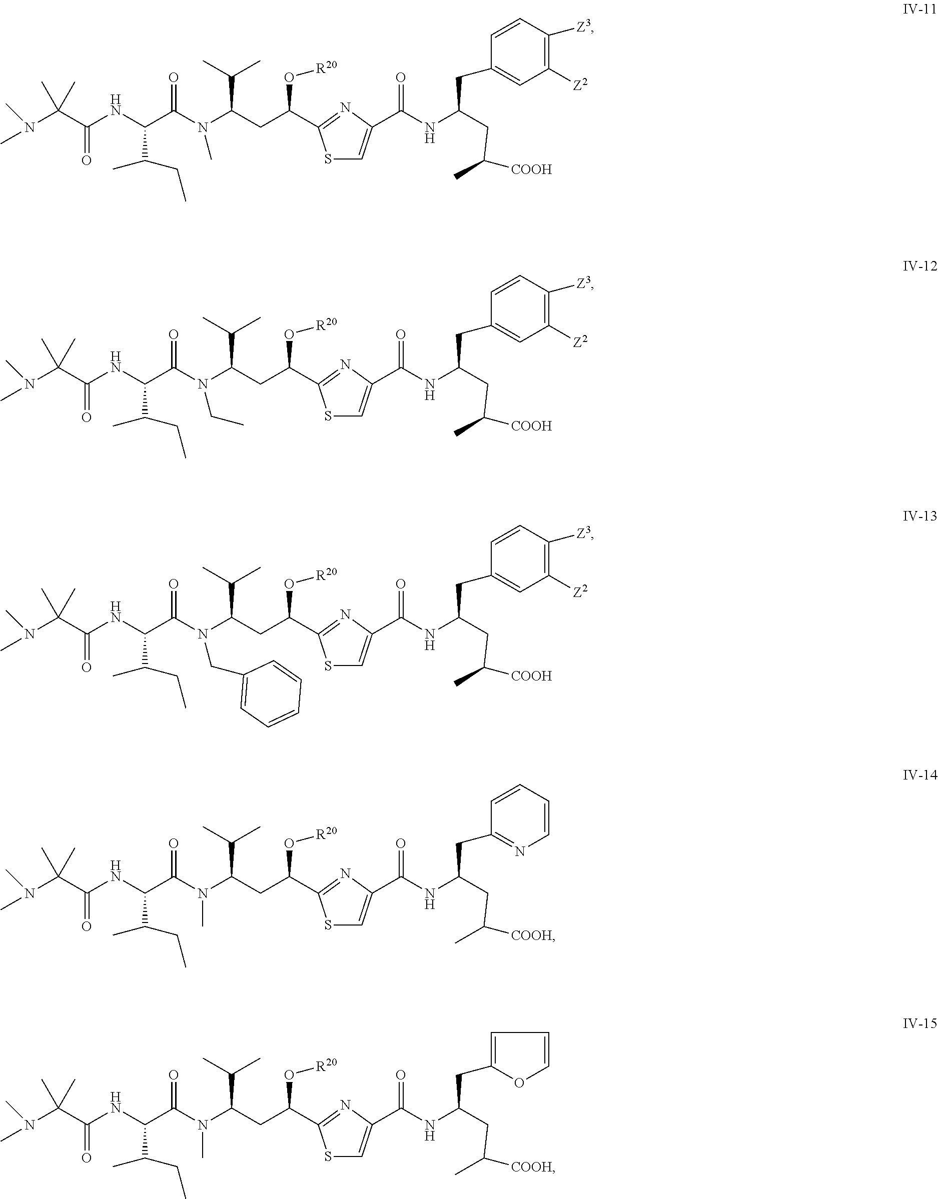 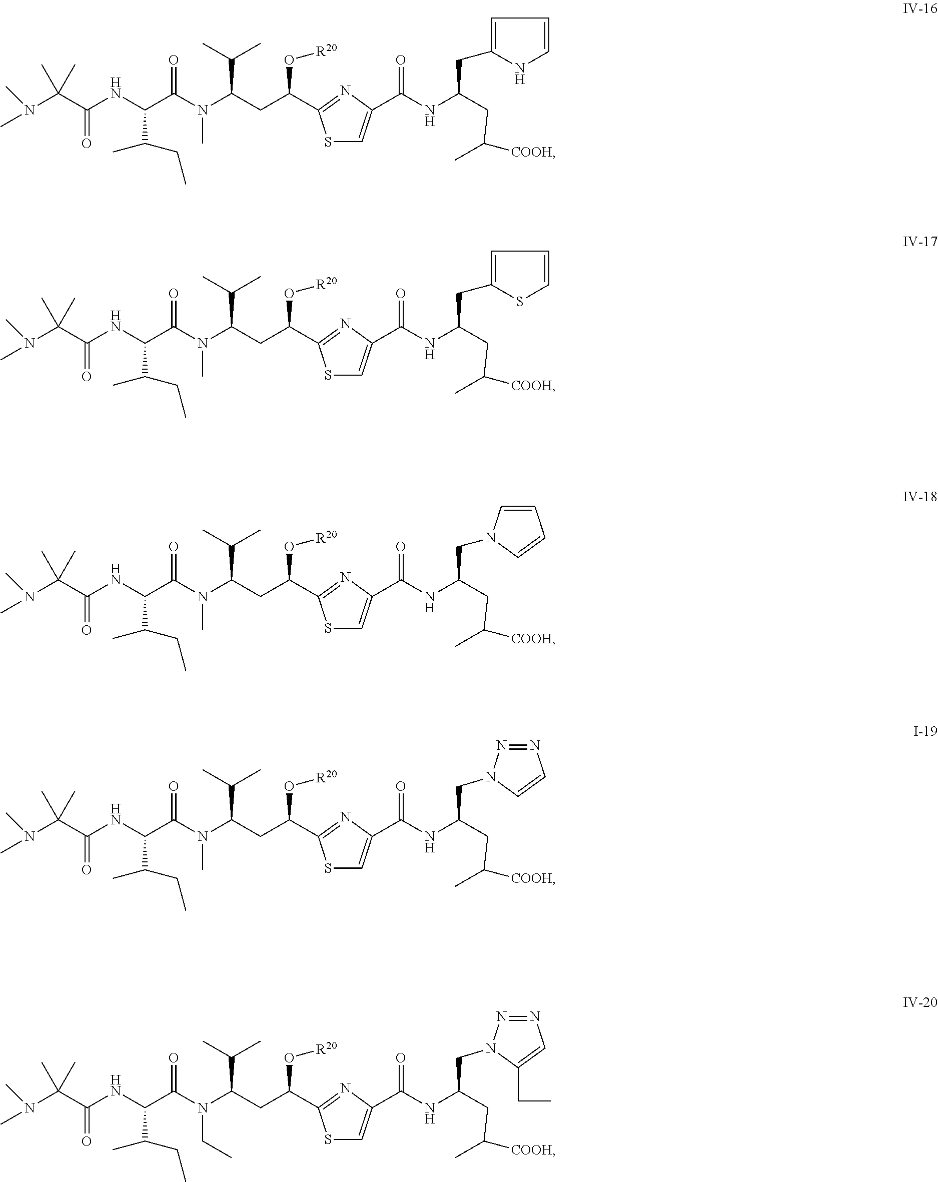 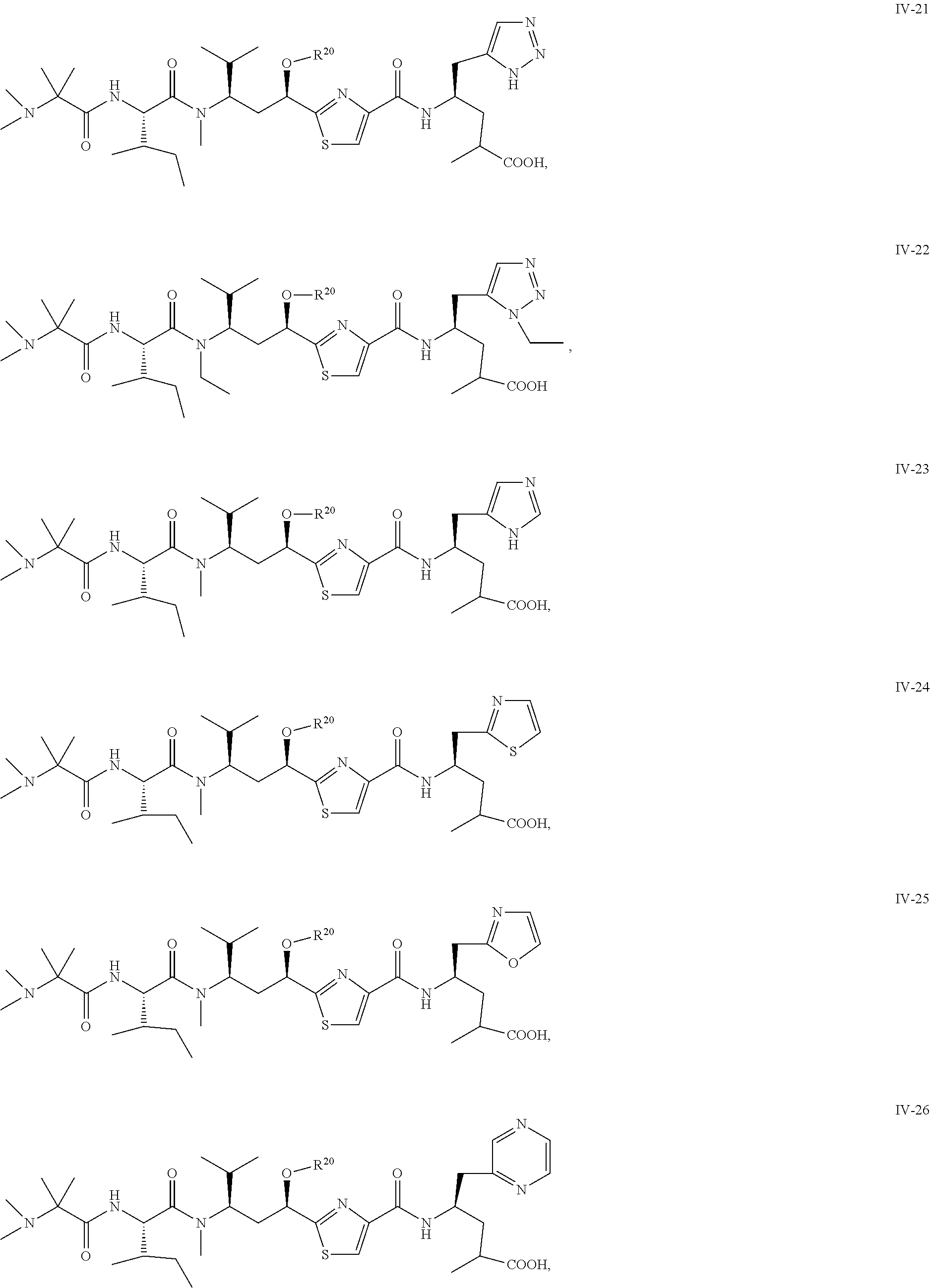 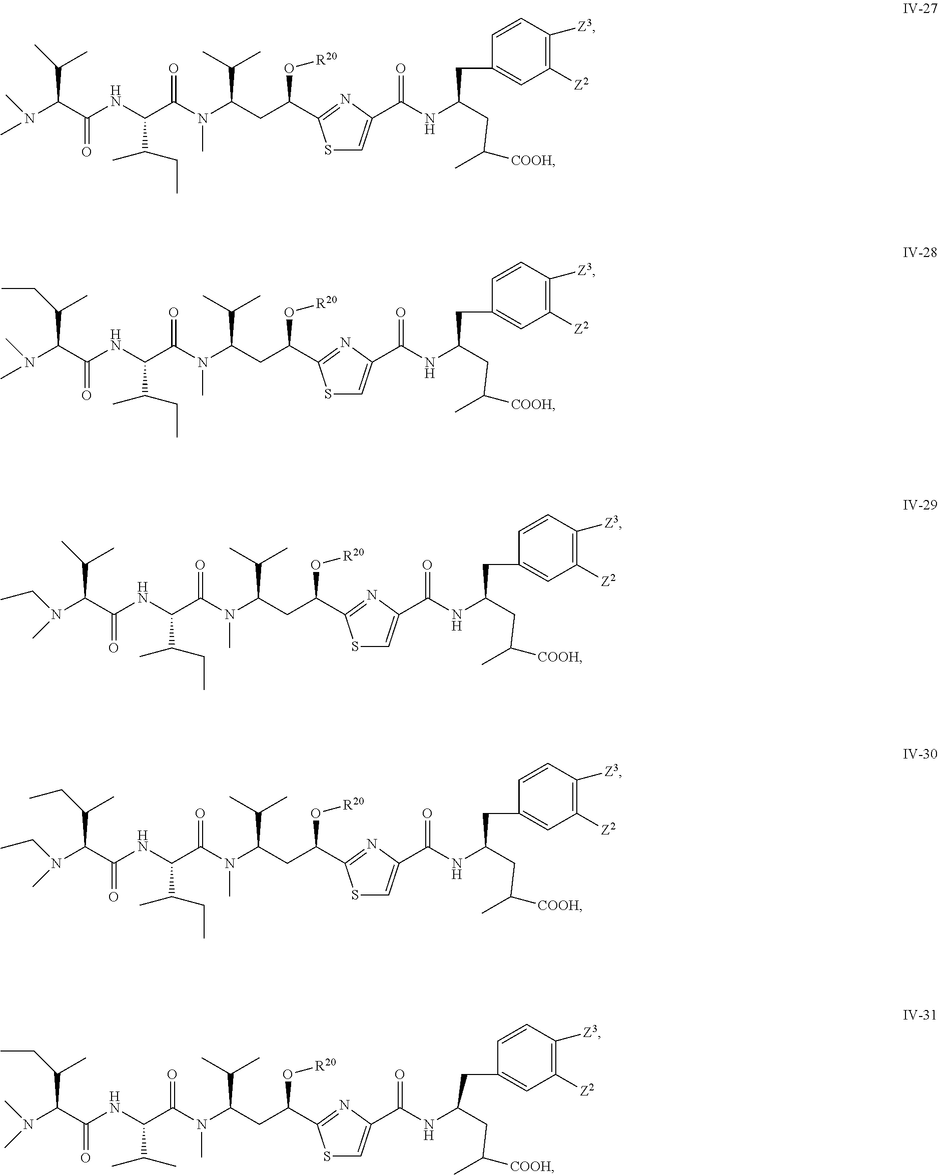 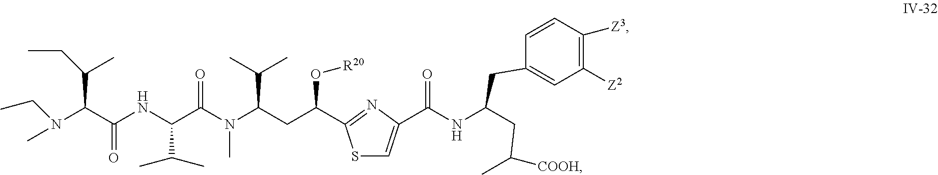 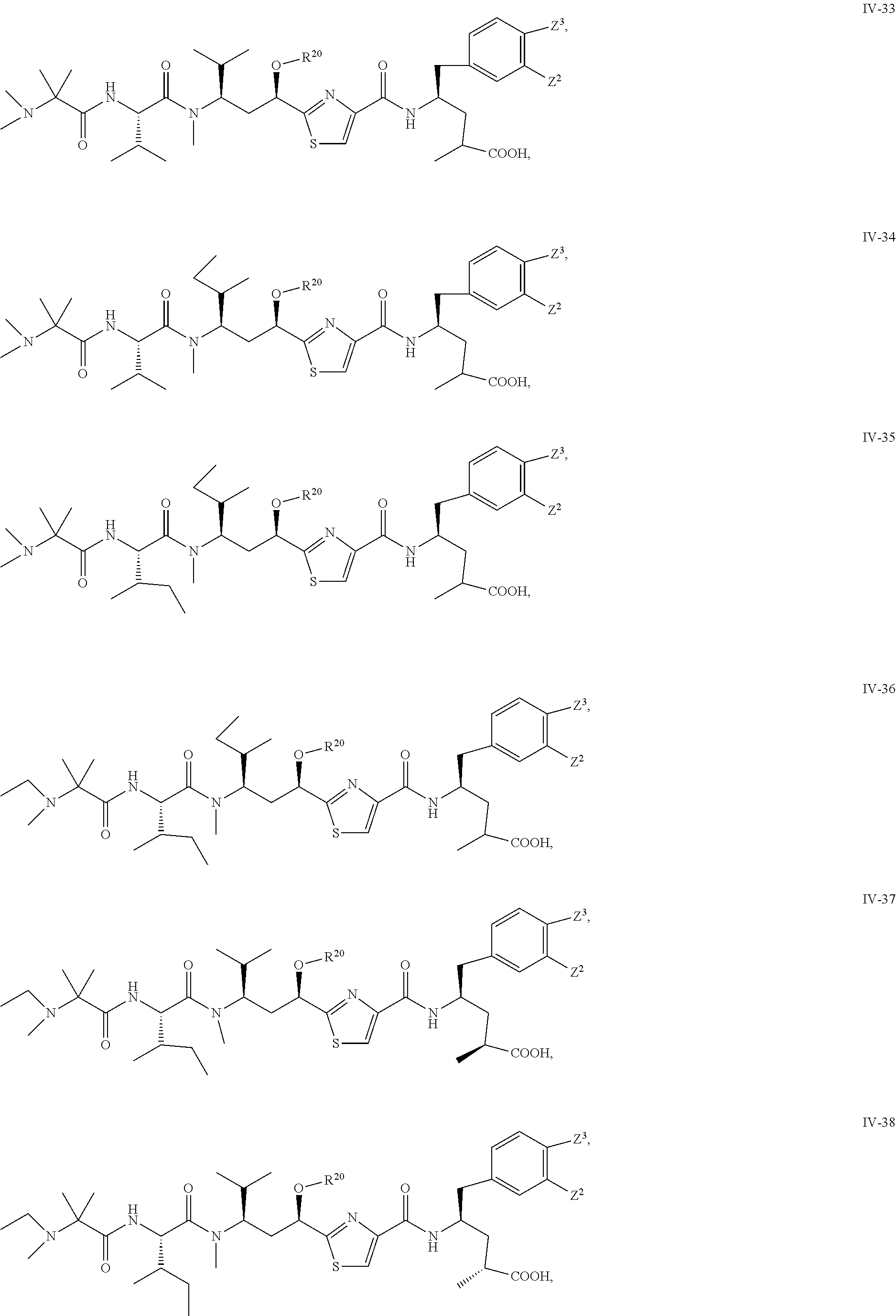 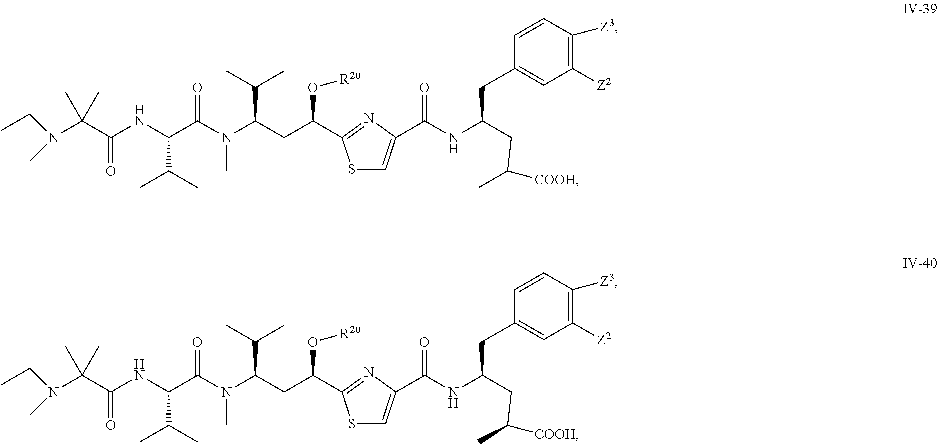 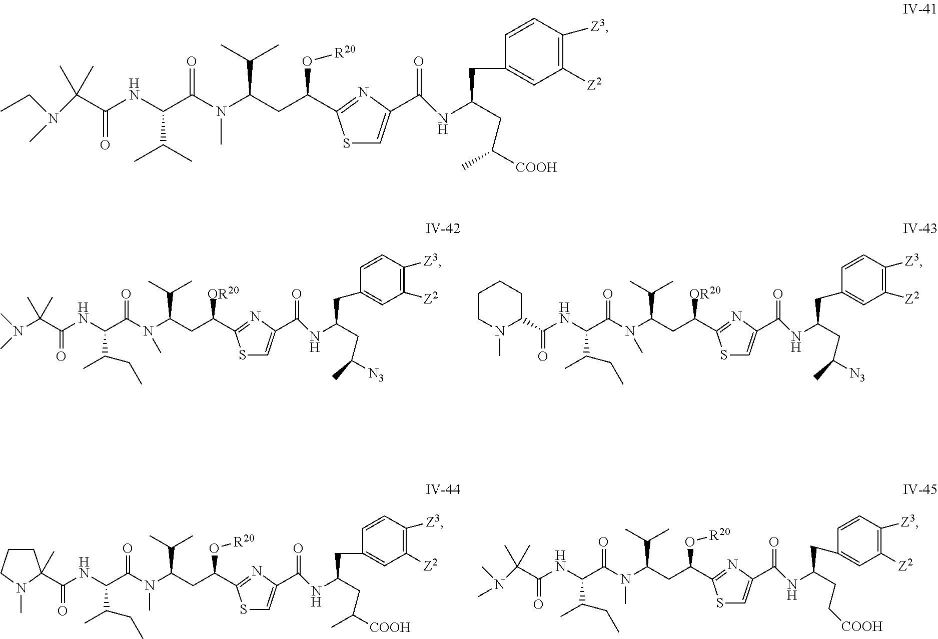 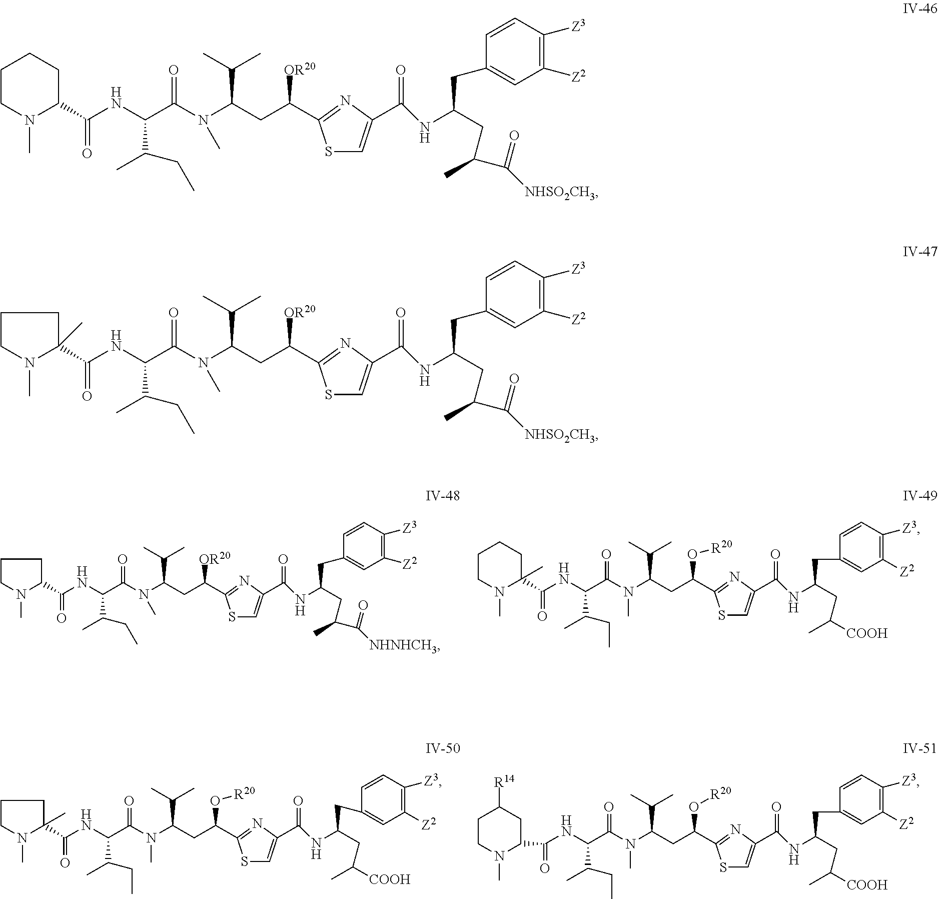 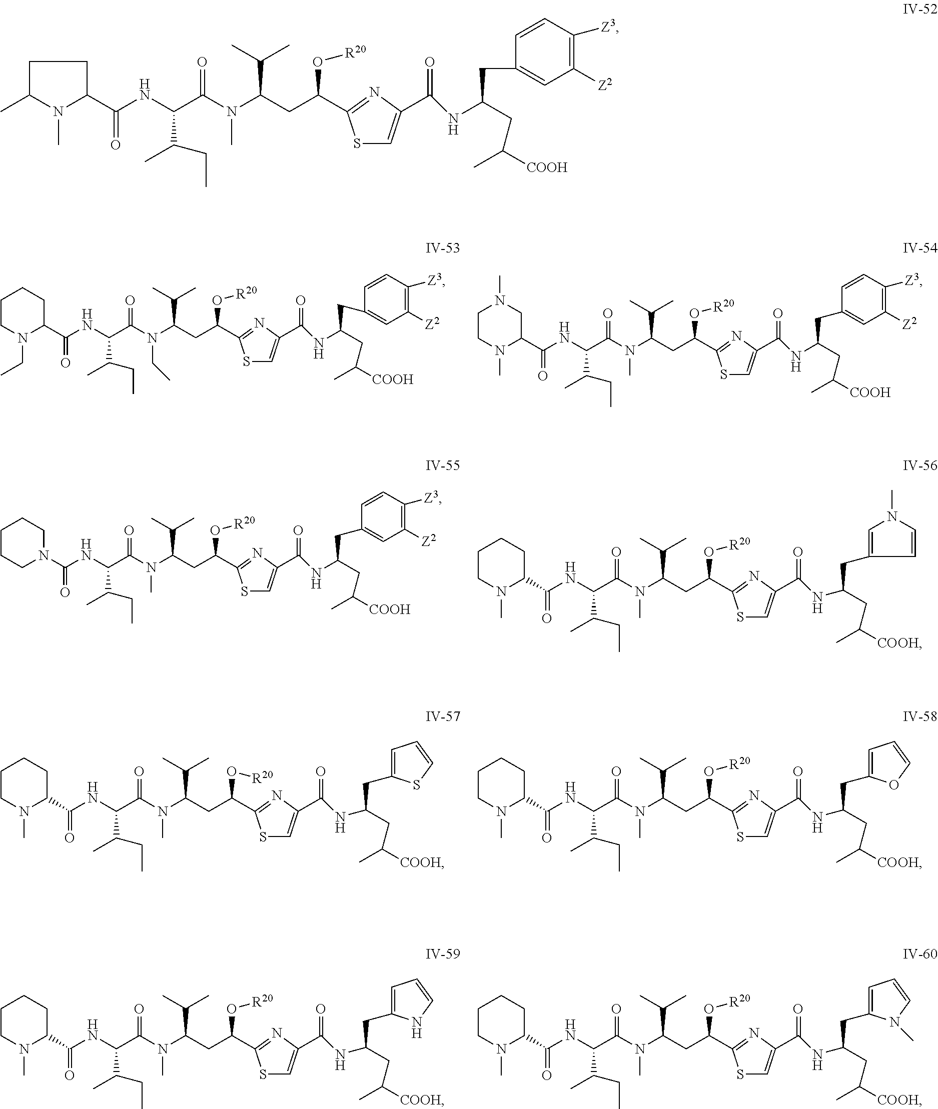 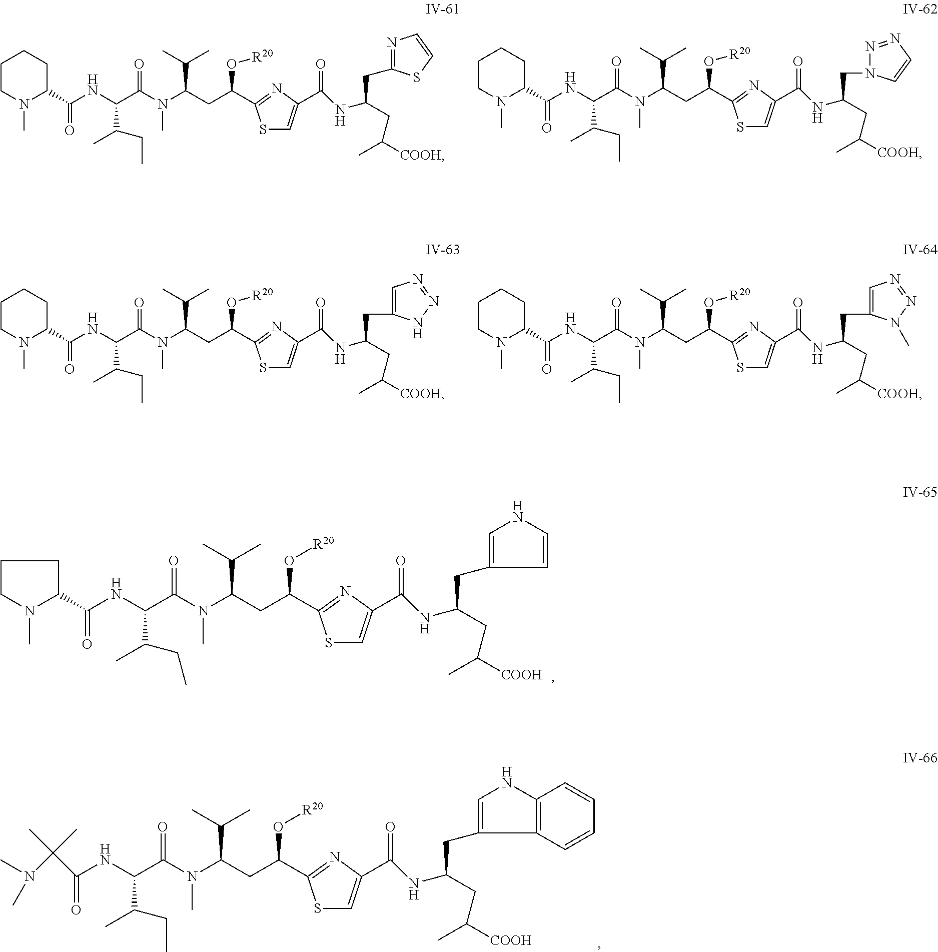 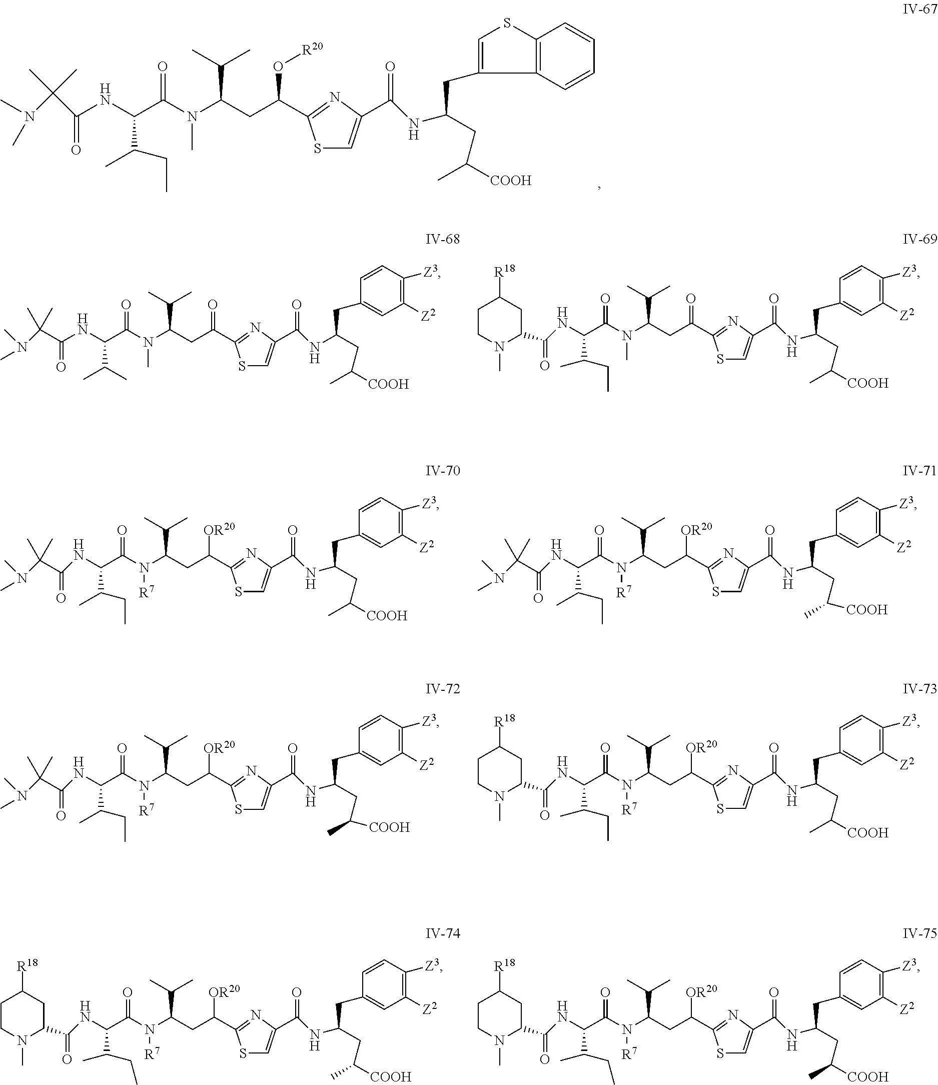 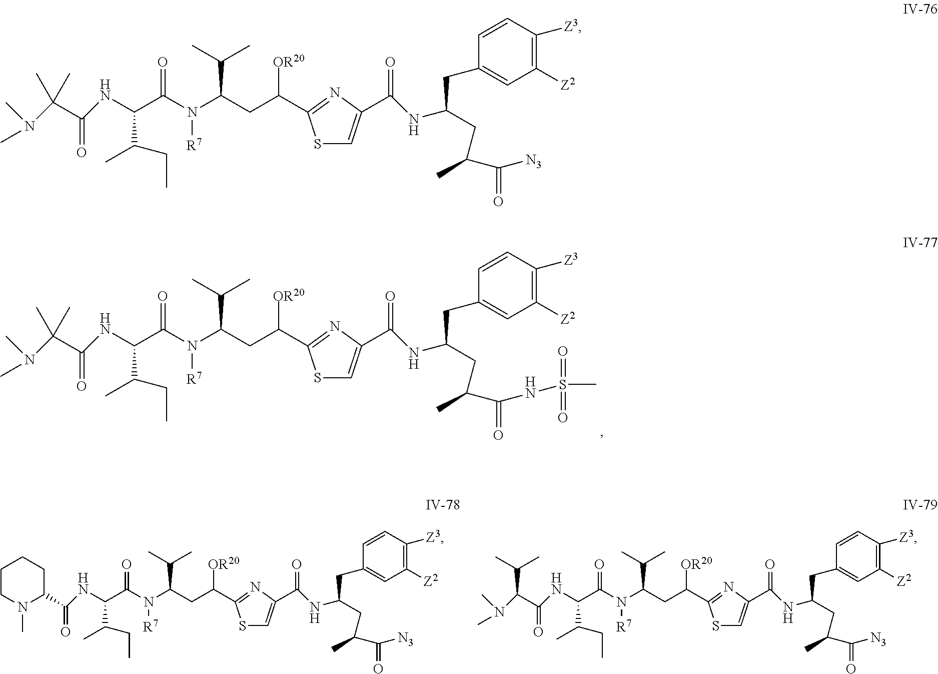 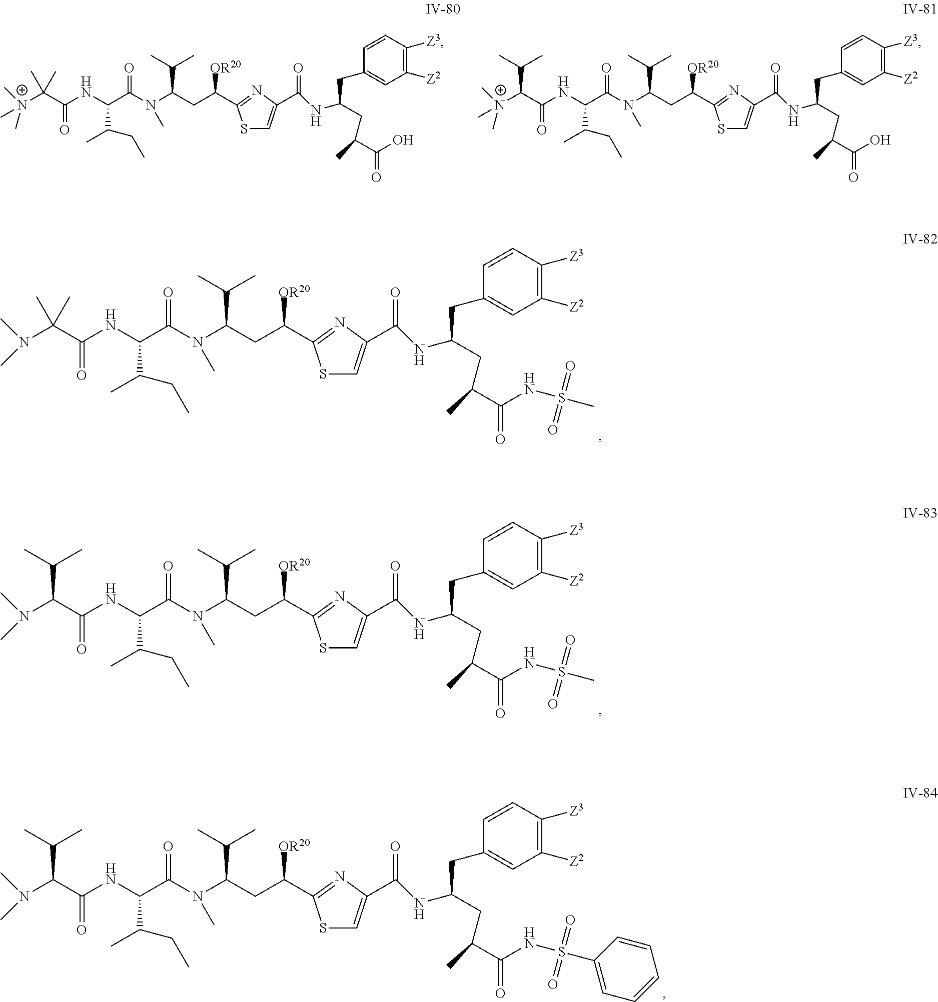 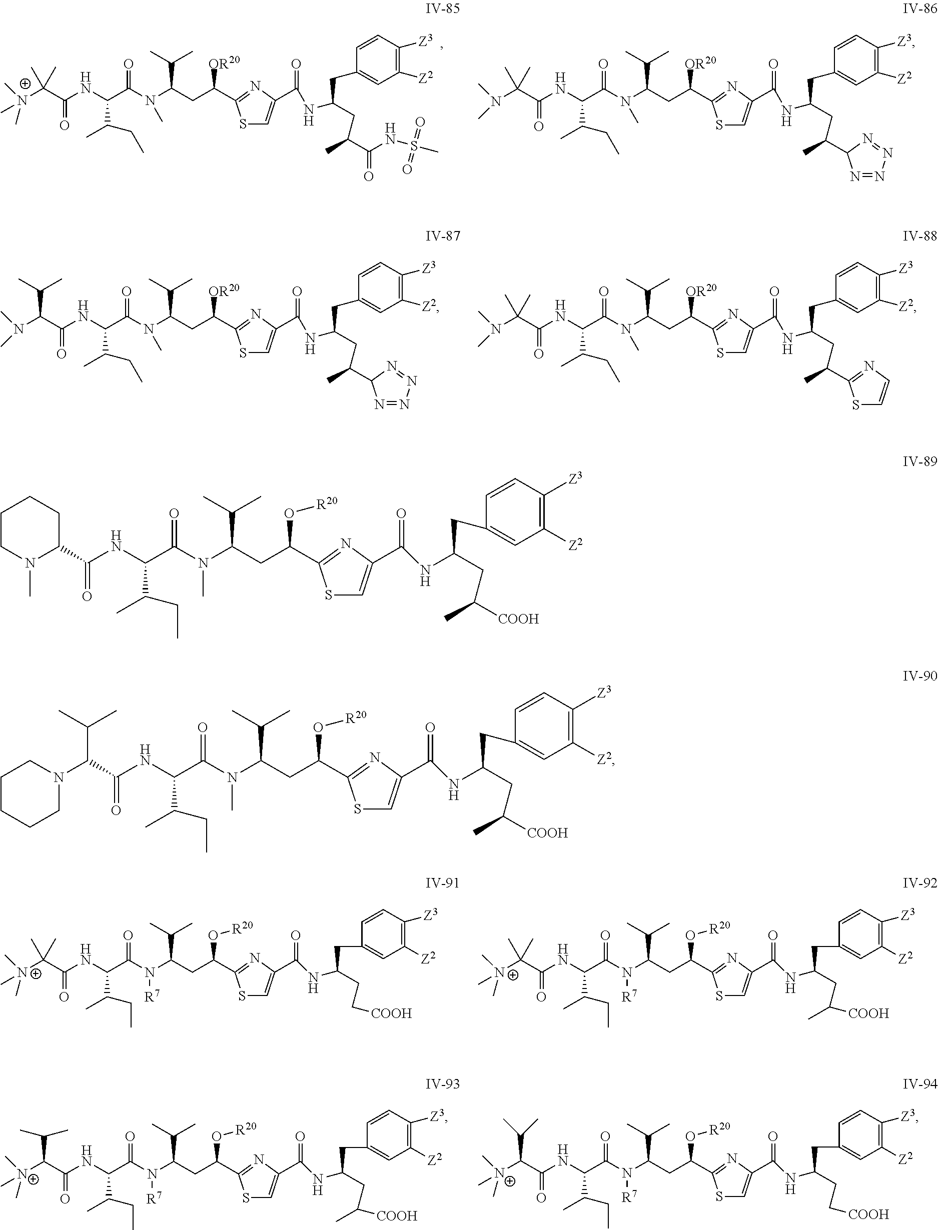
wherein R²⁰ is H; C₁-C₈ of linear or branched alkyl or heteroalkyl; C₂-C₈ of alkylcarbonyl, carbonate (—C(O)OR¹⁷), carbamate (—C(O)NR¹⁷R¹⁸); or 1-8 carbon atoms of carboxylate, esters, ether, or amide; or 1˜8 amino acids; or polyethyleneoxy unit of formula (OCH₂CH₂)ₚ or (OCH₂CH(CH₃))ₚ, wherein p is an integer from 0 to about 100; or R²⁰ is absent and the oxygen forms a ketone, or combination above groups thereof;
R⁷ is independently H, R¹⁴, —R¹⁴C(═O)X¹R¹⁵; or —R¹⁴X¹R¹⁵, X¹ is O, NH, CH₂ or NR¹⁴;
Z² and Z³ are independently H, OH, NH₂, O, NH, COOH, COO, C(O), C(O)NH, C(O)NH₂, R¹⁸, OCH₂OP(O)(OR¹⁸)₂, OC(O)OP(O)(OR¹⁸)₂, OPO(OR¹⁸)₂, NHPO(OR¹⁸)₂, OP(O)(OR¹⁸)OP(O)(OR¹⁸)₂, OC(O)R¹⁸, OC(O)NHR¹⁸, OSO₂(OR¹⁸), O—(C₄-C₁₂-glycoside), carbonate (—C(O)OR¹⁷), carbamate (—C(O)NR¹⁷R¹⁸);
R¹⁴, R¹⁵, R¹⁷ and R¹⁸ are independently H, linear or branched alkyl or heteroalkyl;
R¹⁹ is H, OH, NH₂, OSO₂(OR¹⁸), XCH₂OP(O)(OR¹⁸)₂, XPO(OR¹⁸)₂, XC(O)OP(O)(OR¹⁸)₂, XC(O)R¹⁸, XC(O)NHR¹⁸, C₁˜C₈ alkyl or carboylate; or pharmaceutical salts;
X is O, S, NH, NHNH, or CH₂.

6. The antibody drug conjugate according to claim 1, wherein the cytotoxic drug, D₁ and D2, are independently selected from: calicheamicin (Ia), Geldanamycins (I2-1, and I2-2), maytansinoid (Ib), cryptophycin (I3-1, I3-2, I3-3, I3-4, I3-5, I3-6, I3-7 and I3-8), Combretastatins (CA-01, CA-02 and CA-03), Taxanes (Id-01, Id-02, Id-03 and Id-04), having the following formula: 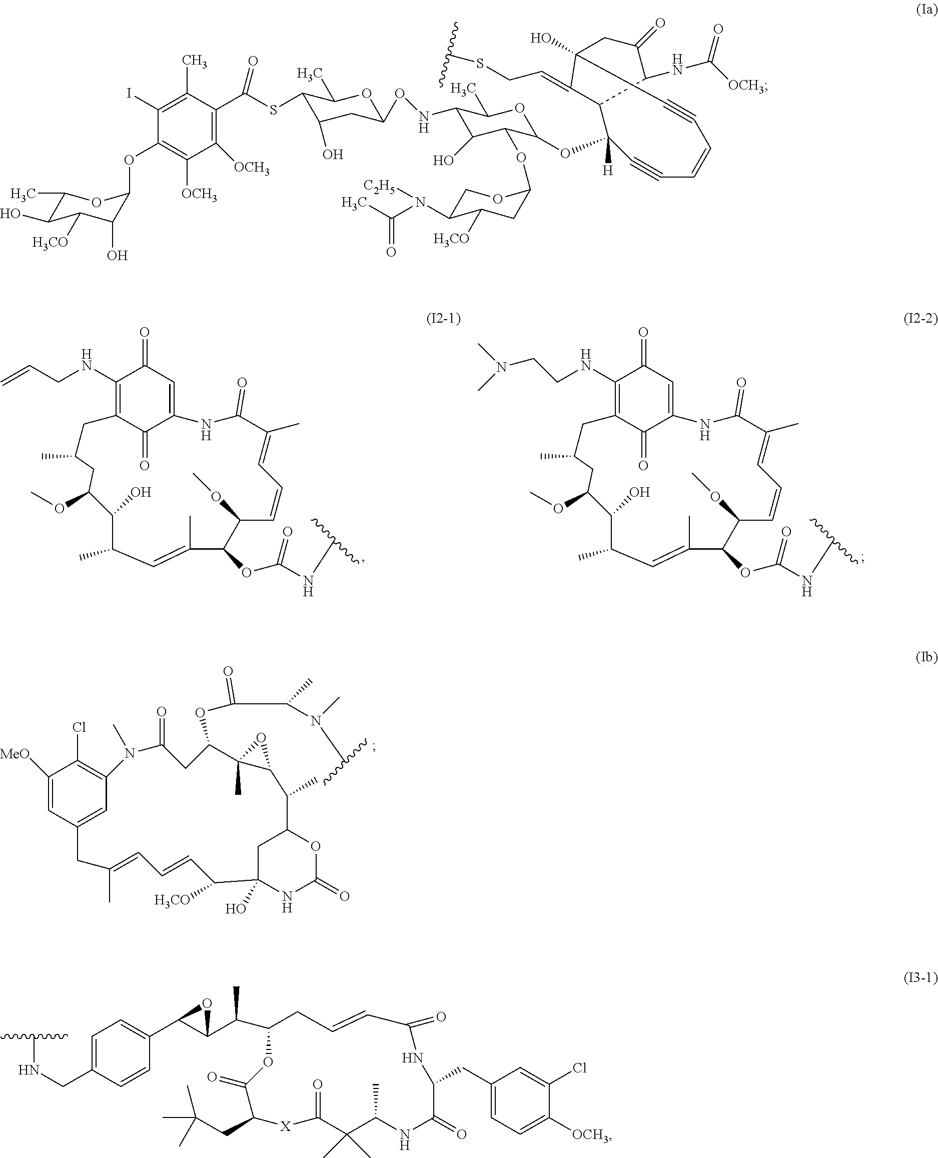 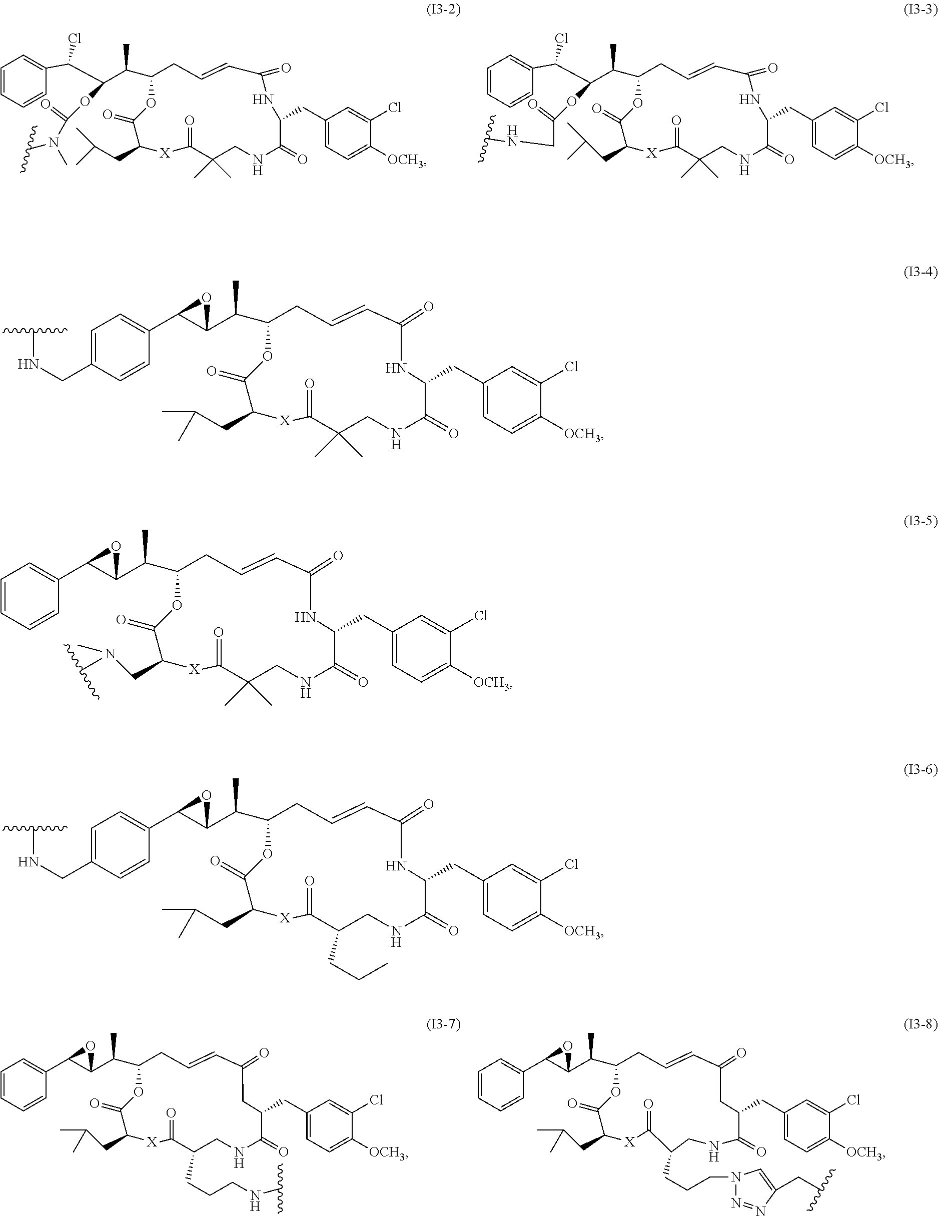 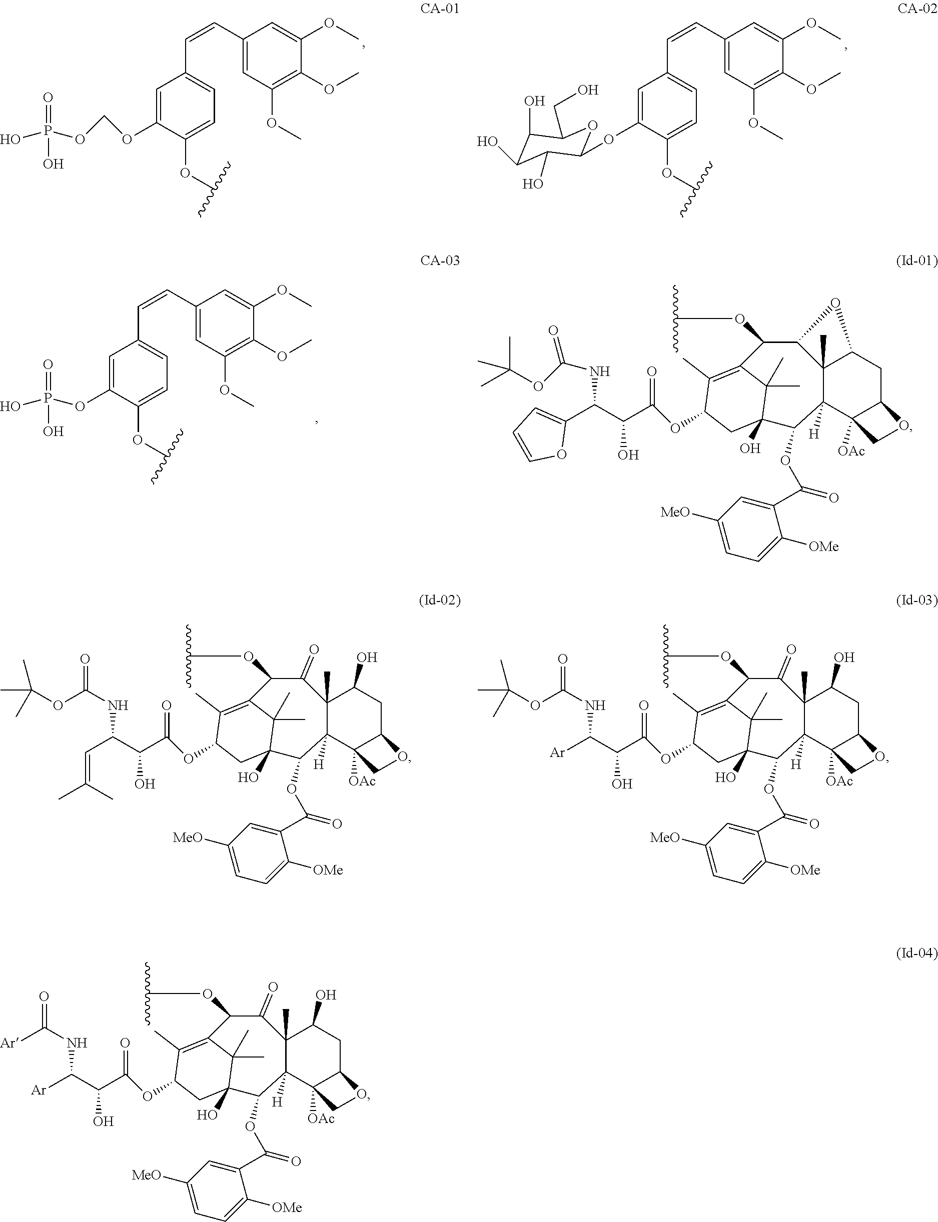
or an isotope of a chemical element, or a pharmaceutically acceptable salt, hydrates, or hydrated salt; or a polymorphic crystalline structure; or an optical isomer, racemate, diastereomer or enantiomer thereof,
wherein X is O or NH; Ar and Ar′ are independently aryl or heteroaryl and [symbol] is the site linked to L₁ or L₂.

7. The antibody drug conjugate according to claim 1, wherein the cytotoxic drug, D₁ and D₂, are independently selected from: Camptothecin (CPT) having the following formula (Ic): 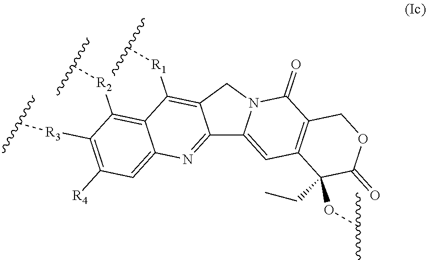
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein R₁, R₂ and R₄ are independently selected from H, F, Cl, Br, CN, NO₂, C₁˜C₈ alkyl; O-C₁˜C₈ alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or C₂-C₈ of esters, ether, amide, carbonate, urea, or carbamate; R₃ is H, OH, NH₂, C₁˜C₈ alkyl; O-C₁˜C₈ alkyl; NH—C₁˜C₈ alkyl; C₂-C₈ of heteroalkyl, alkylcycloalkyl, heterocycloalkyl, O-C₂˜C₈ alkylamine, NH—C₂˜C₈ alkylamine, O-C₂˜C₈ alkylalcohol, NH—C₂˜C₈ alkylalcohol; or C₂˜C₈ (2-8 carbon atoms) of esters, ether, amide, carbonate, urea, or carbamate; or R₁R₂, R₂R₃ and R₃R₄ independently form a 5˜7 membered carbocyclic, heterocyclic, heterocycloalkyl, aromatic or heteroaromatic ring system; [symbol] is the site in the molecule that can be linked to L₁ or L₂.

8. The antibody drug conjugate according to claim 1, wherein the cytotoxic drug, D₁ and D₂, are independently selected from: Camptothecin (CPT) having the following formula: 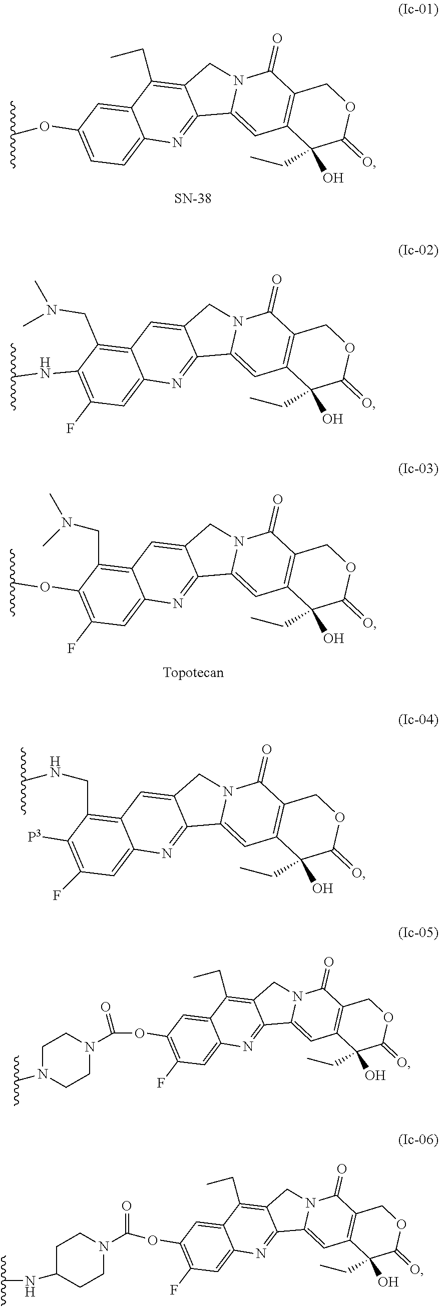 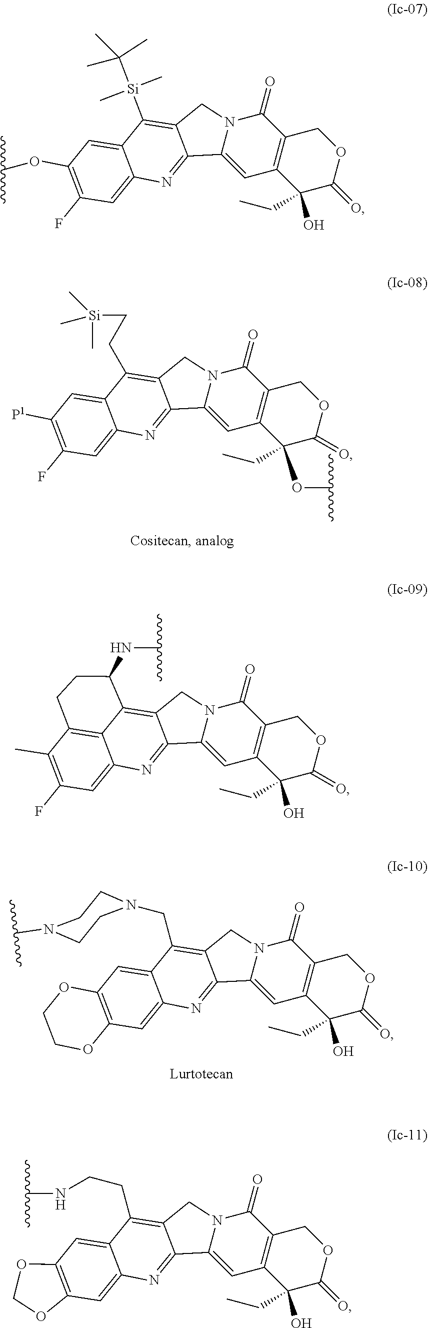 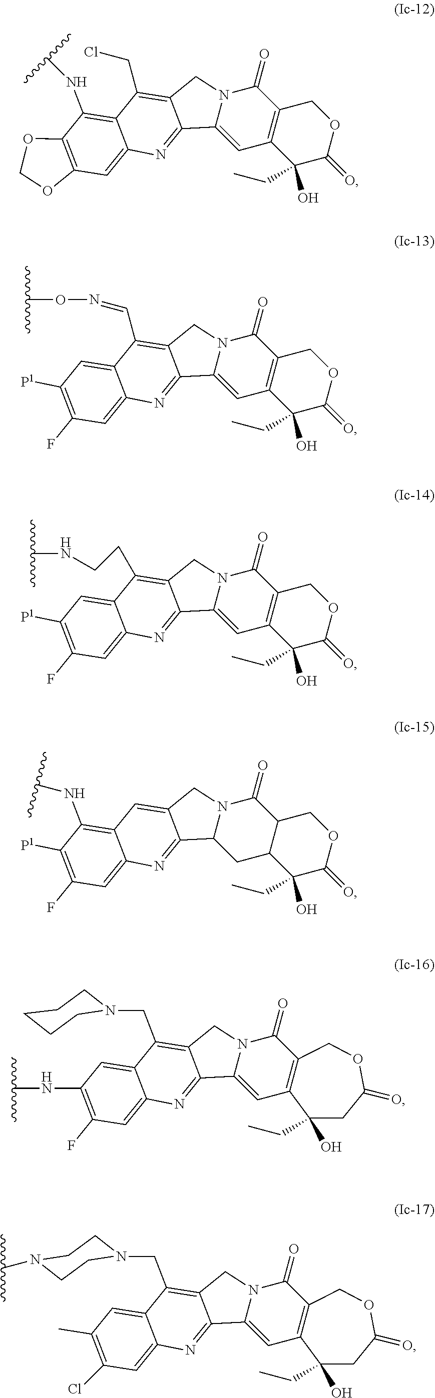 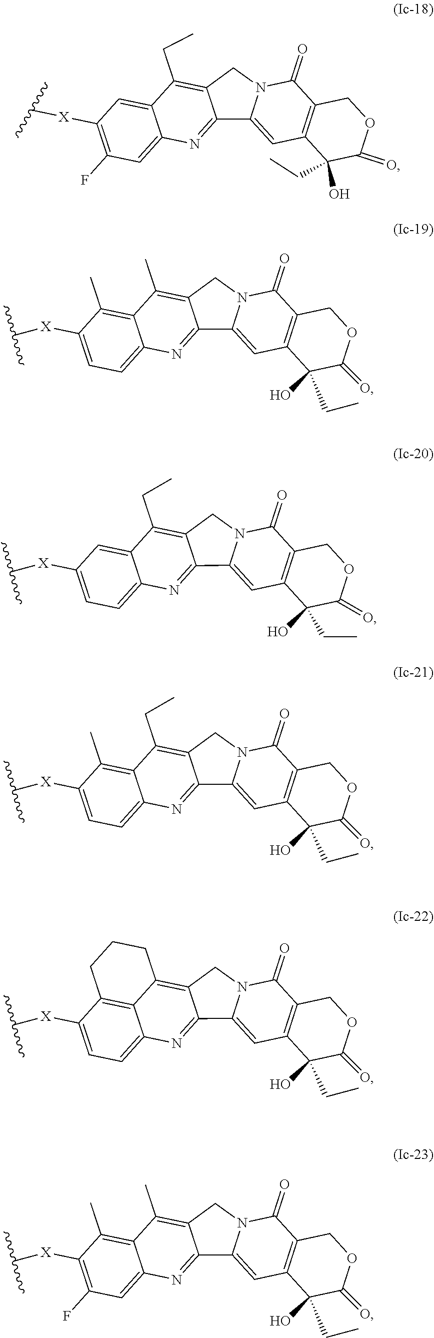 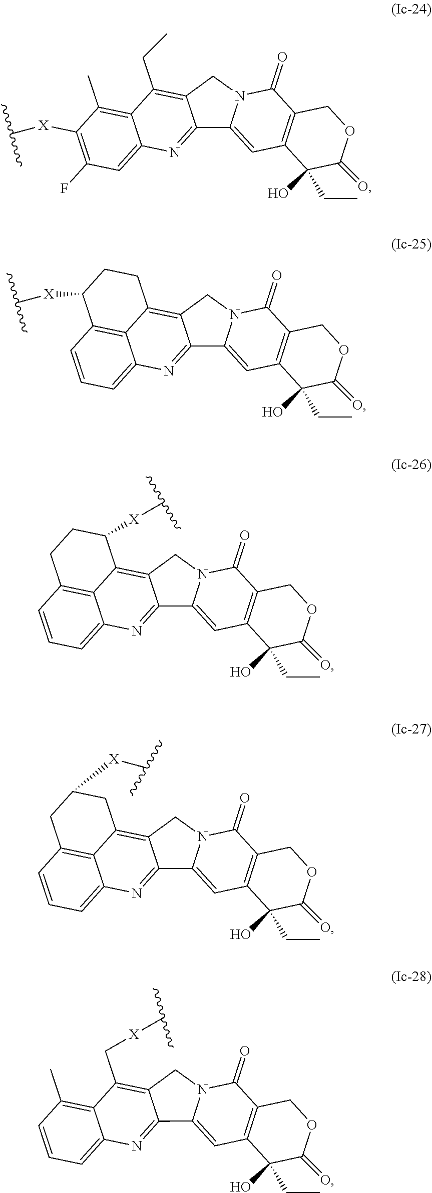 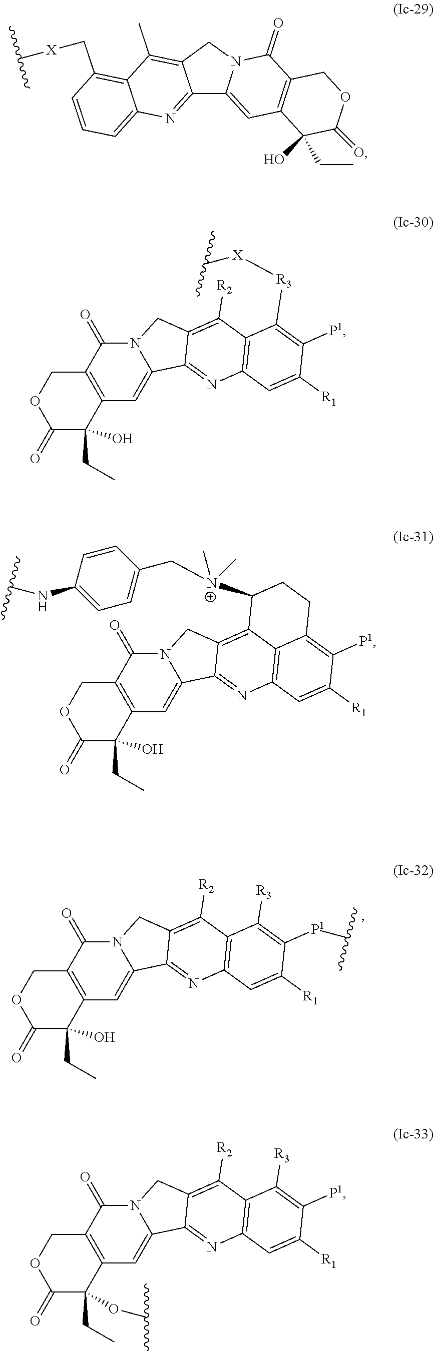 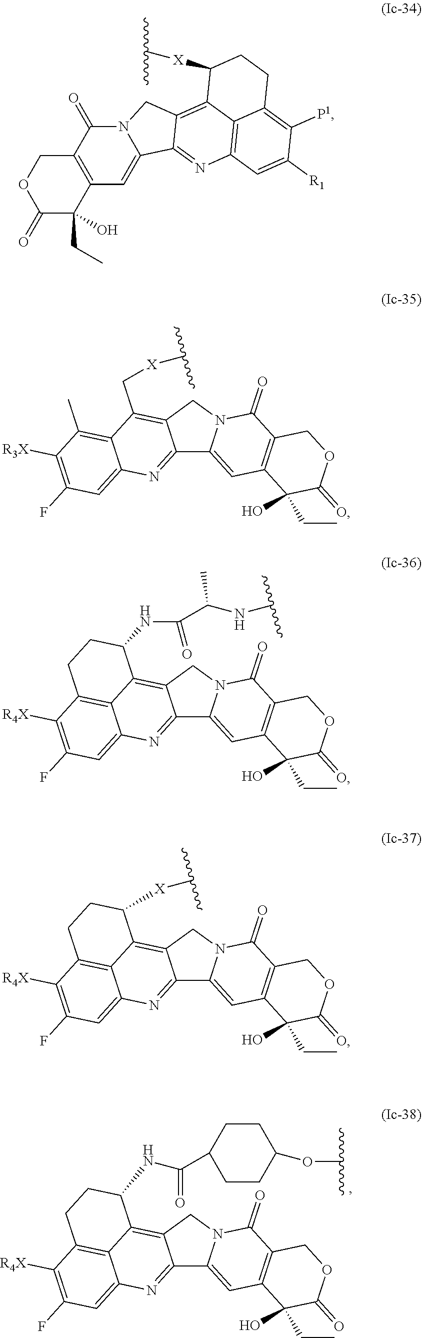 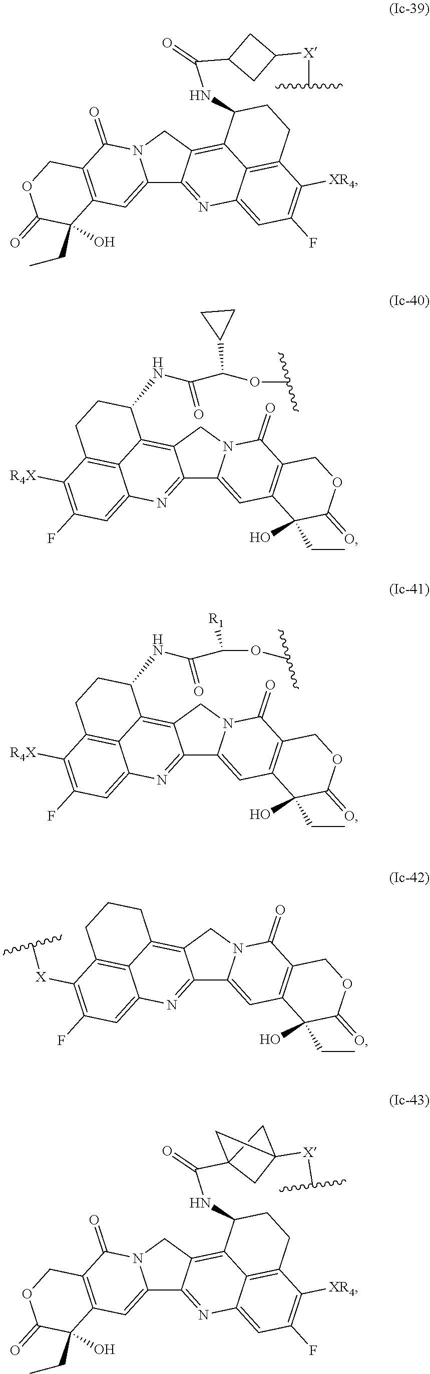 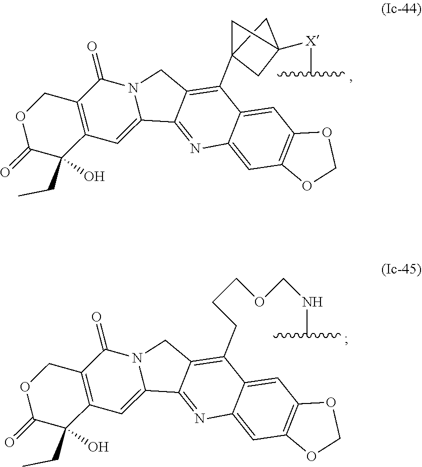
or hydrated salts; or the polymorphic crystalline structures of these compounds; or the optical isomers, racemates, diastereomers or enantiomers; wherein [symbol] is the site linked to L₁ or L₂;
wherein P¹ and R₁ are independently H, F, Cl, Br, I, SO₂R₂, SO₃H, CN, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸ OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, O—(C₁-C₈ of linear or branched alkyl), O—(C₁-C₈ of linear or branched alkyl)-OH, C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸);
R₂, R₃ and R₄ are independently H, C₁-C₈ of linear or branched alkyl, or heteroalkyl, C₂-C₈ of linear or branched ether, amine, ester, or amide; or R₂ and R₃ can be joint together to form five or six member ring or cycloalkyl, cycloalkylamine, or cycloalkylamide, cyclohexylalkylamide; R₁₇ and R₁₈ are independently H, linear or branched alkyl or heteroalkyl;
X is NH, O, S, S(O₂), NHS(O₂), NHS(O₂)NH, NHP(O)(OH), N⁺(R₂) (R₃), NHC(O)NH, NHC(O), NHC(O)O, N(CH₂CH₂)₂N, CON(CH₂CH₂)₂N, CON(CH₂CH₂)₂N⁺(R₂)(R₃), NHCH₂, N(CH₃), OCH₂, or CH₂; X′ is O, NH, CH₂, CH₂NH, or CH₂O.

9. The antibody drug conjugate according to claim 1, wherein the cytotoxic drug, D₁ and D2, are independently selected from, Anthracycline (Ie-01, Ie-02, Ie-03, Ie-04, Ie-05, Ie-06, Ie-07, Ie-08, Ie-09, Ie-10, Ie-11, Ie-12, Ie-13)), Vinca alkaloid (If-01, If-02, If-03, If-04, If-05, and If-06), Eribulin (Eb01), spliceostatin (Sp-01) and proteinase inhibitor (PI01, PI02 and PI03) having the following formula: 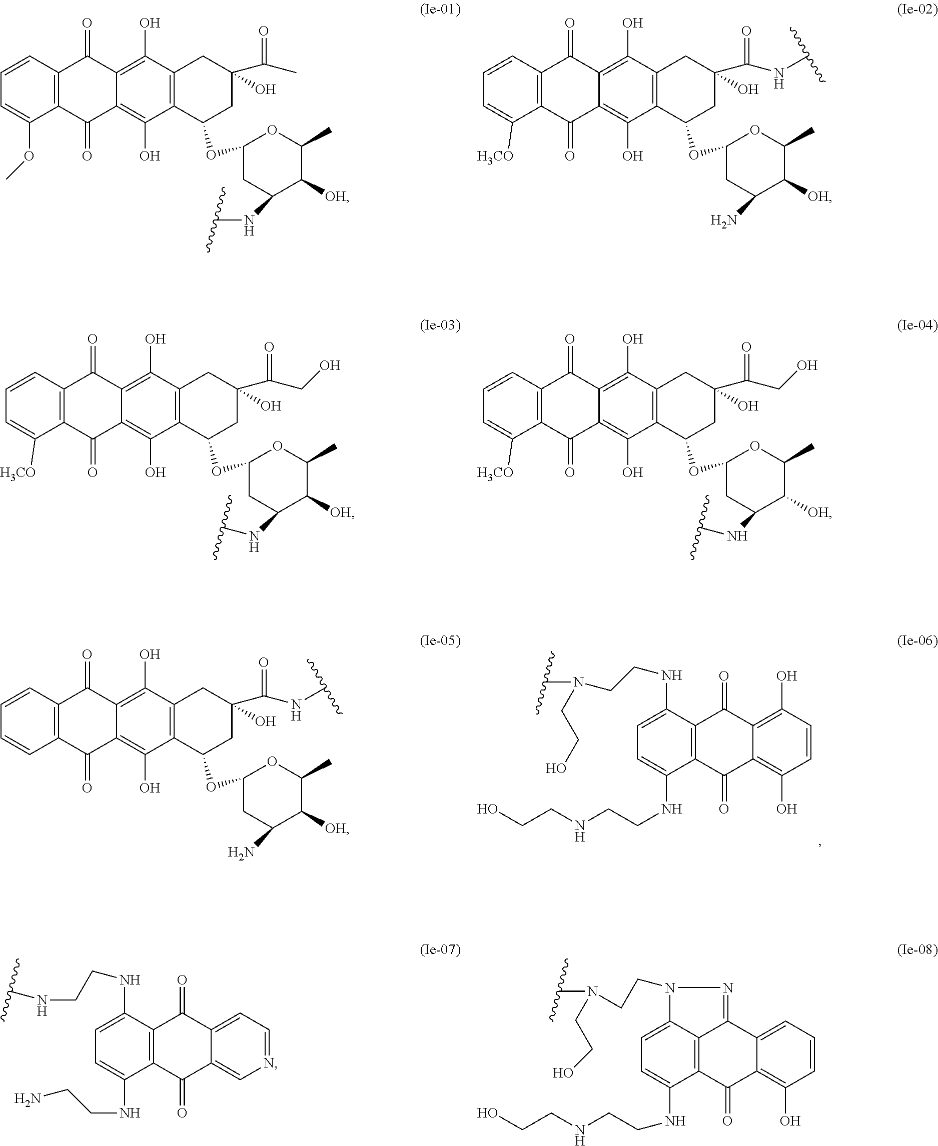 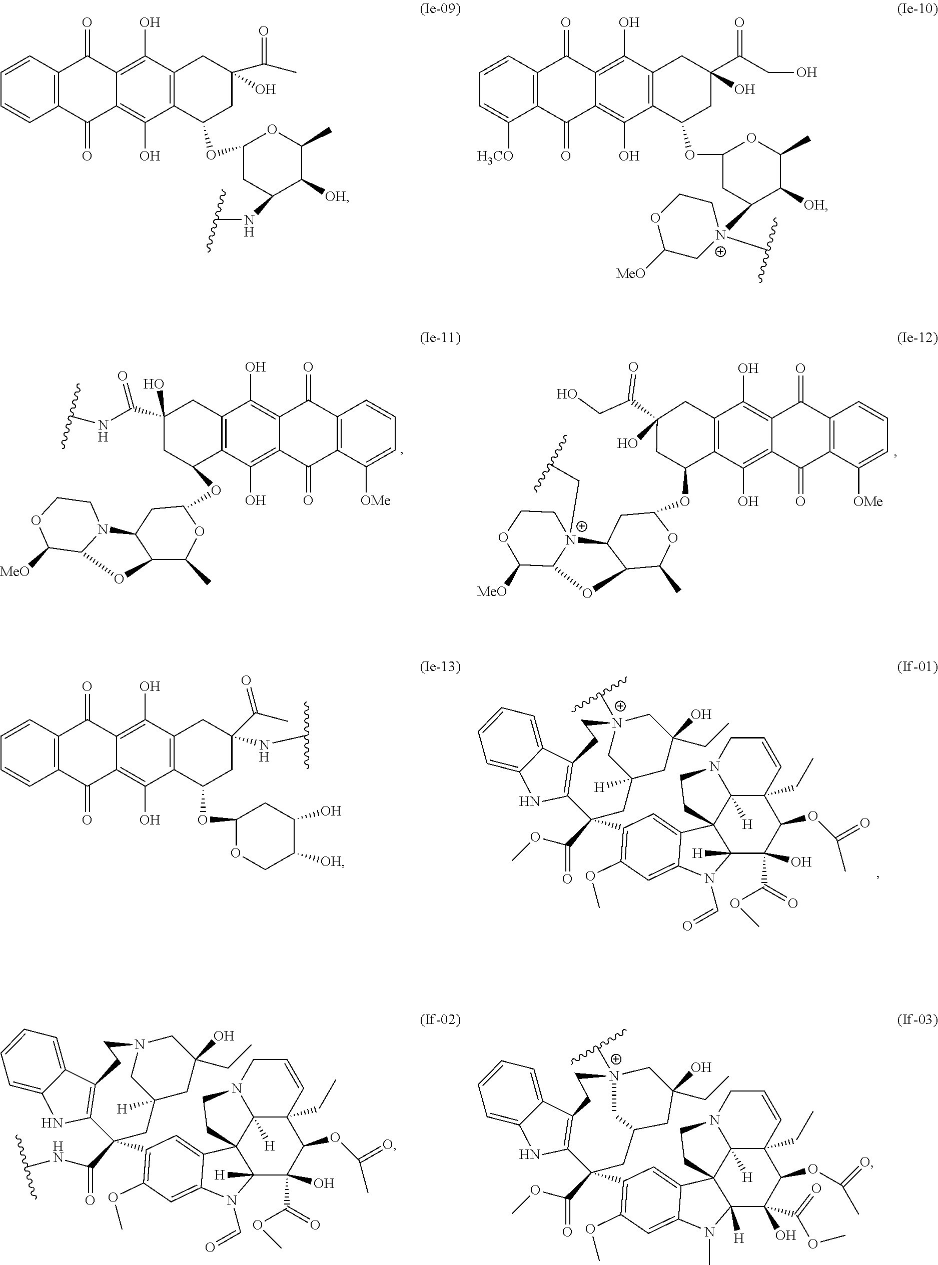 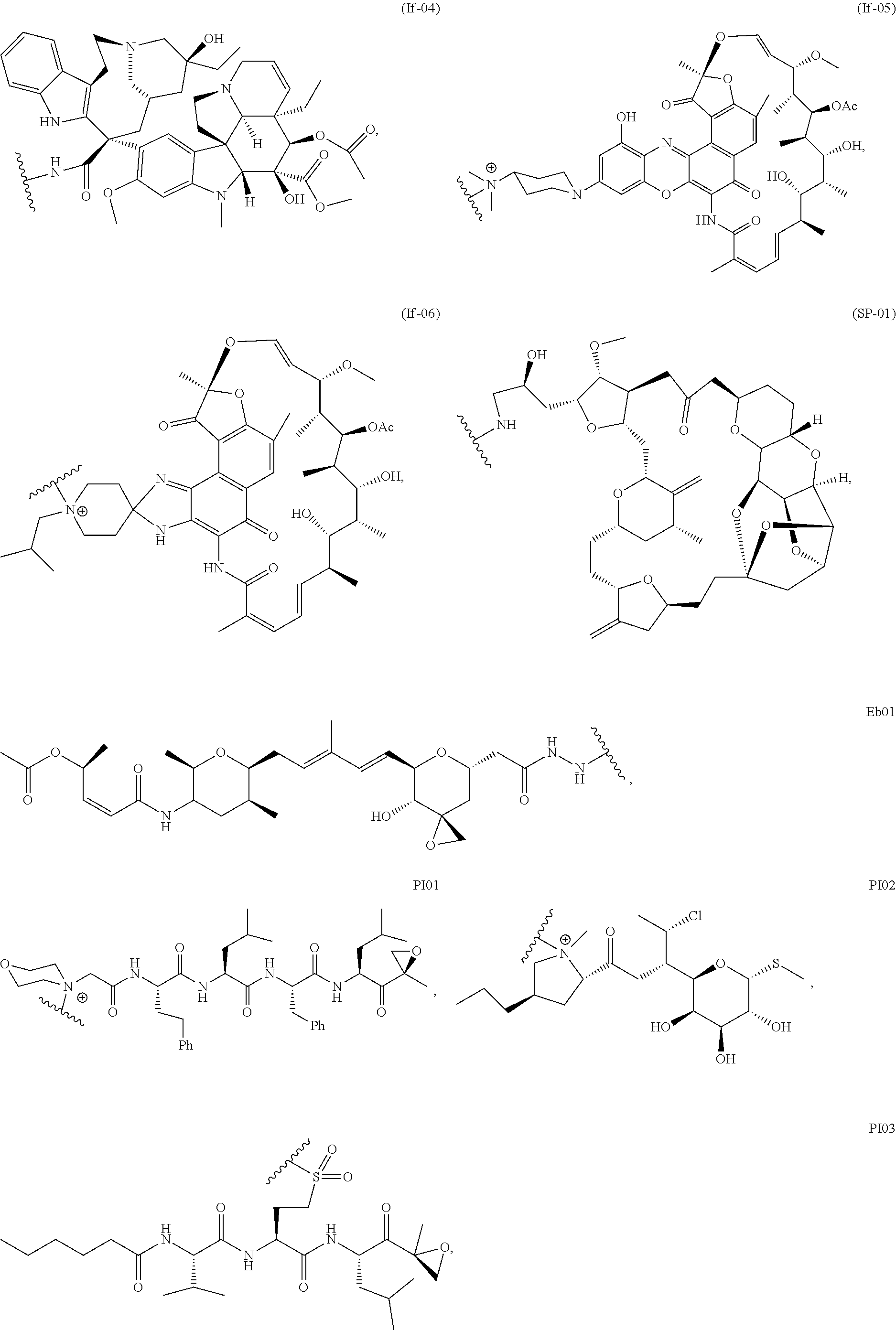
or an isotope of one or more chemical elements, or pharmaceutically acceptable salts, hydrates, or hydrated salts; or the polymorphic crystalline structures of these compounds; or the optical isomers, racemates, diastereomers or enantiomers, wherein [symbol] is the site that links to L₁ or L₂.

10. The antibody drug conjugate according to claim 1, wherein the cytotoxic drug, D₁ and D2, are independently selected from, Dolastatins and their peptidic analogs, having the following formula (Ih-01), (Ih-02), (Ih-03), (Ih-04), (Ih-05), (Ih-06), (Ih-07), (Ih-08), (Ih-09), (Ih-10), and (Ih-11): 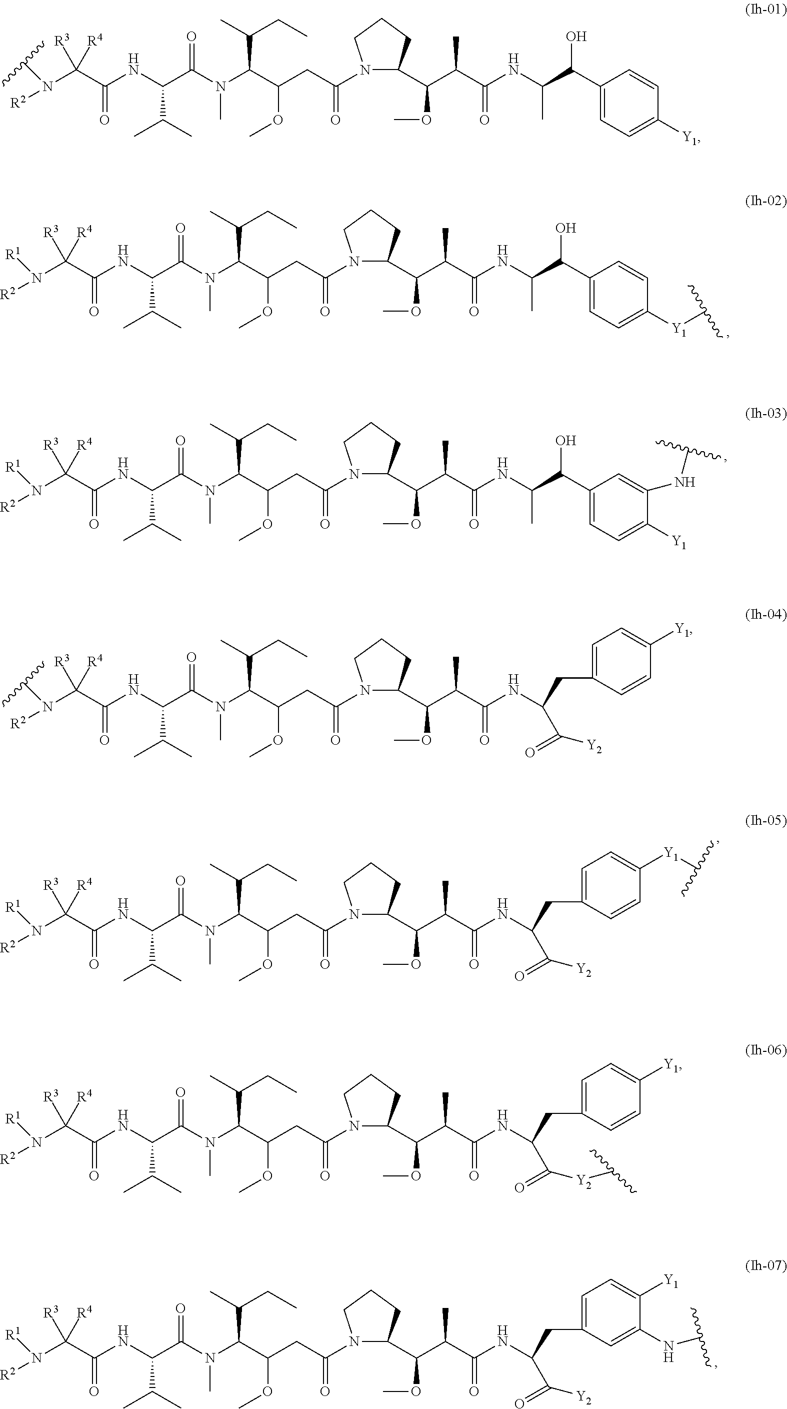 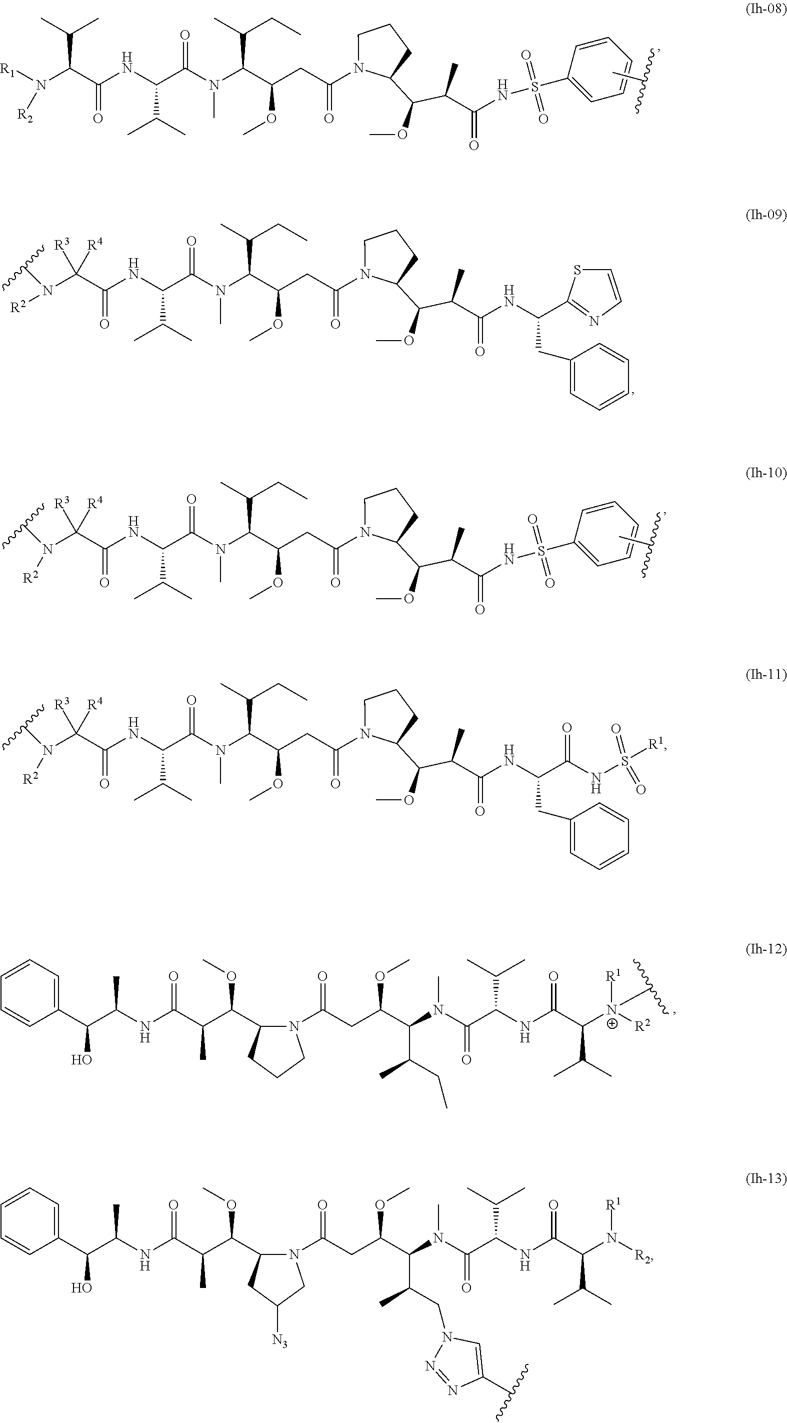 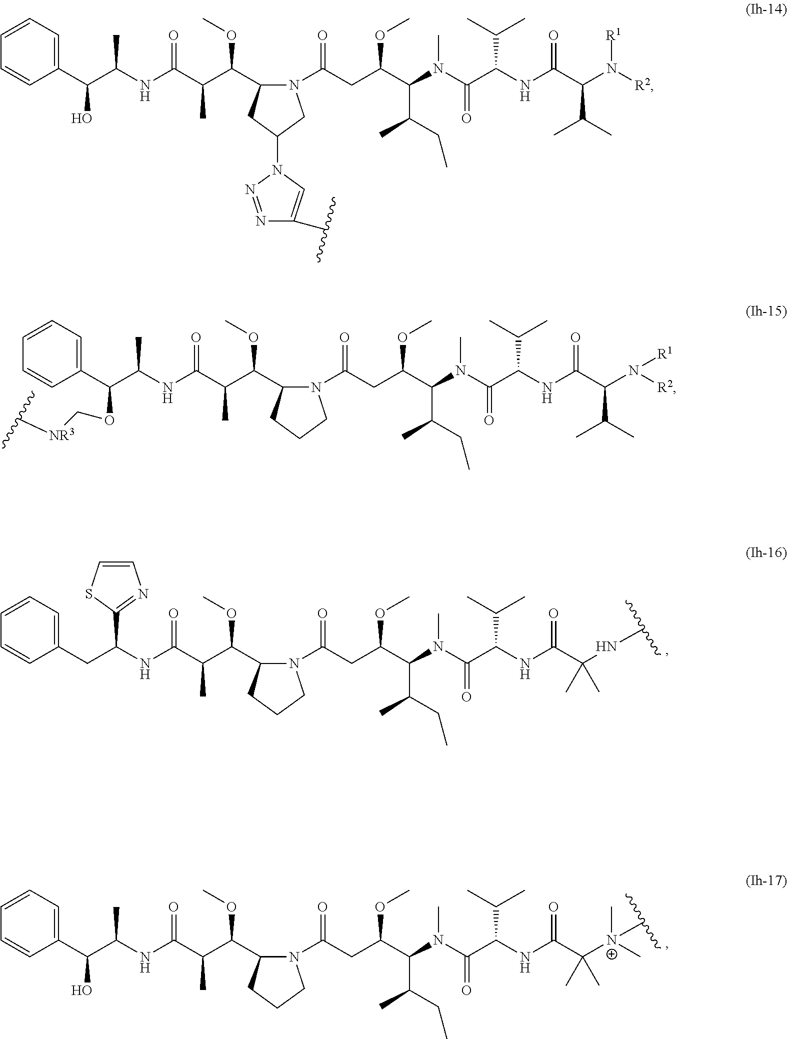
or hydrated salts; or the polymorphic crystalline structures of these compounds; or the optical isomers, diastereomers or enantiomers;
wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃)) p, wherein p is an integer from 1 to about 100, two Rs: R¹R², R²R³, R¹R³ or R³R⁴ together can form 3˜8 member cyclic ring of alkyl, aryl, heteroaryl, heteroalkyl, or alkylcycloalkyl group; Y1 and Y2 are independently O, NH, NHNH, NR⁵, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR¹ when linked to the connecting site “ [symbol] ” (that links to L₁ and/or L₂ independently); or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; R₁₂ is OH, NH₂, NHR₁, NHANH₂, NHANHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR¹R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR¹, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-100; R₁, R₂, R₃, R₄, R₅, R₅′, Z¹, Z², and n are defined the same above.

11. The antibody drug conjugate according to claim 1, wherein the cytotoxic drug, D1 and D2, are independently selected from, Hemiasterlin and their peptidic analogs, having the following formula (Hs-01 and Hs-02): 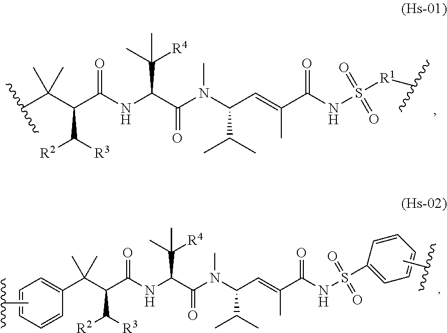
wherein R¹, R², R³, R⁴ and R⁵ are independently H; C₁-C₈ linear or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 100; or R₂R₃ can form 3˜8 member cyclic ring of alkyl, aryl, heteroaryl, heteroalkyl, or alkylcycloalkyl group; [symbol]
is a linkage site that links to L₁ and/or L₂ independently.

12. The antibody drug conjugate according to claim 1, wherein the cytotoxic drug, D₁ and D₂, are independently selected from, an Inhibitor of nicotinamide phosphoribosyltransferases (NAMPT), having the following formula NP01, NP02, NP03, NP04, NP05, NP06, NP07, NP08, and NP09: 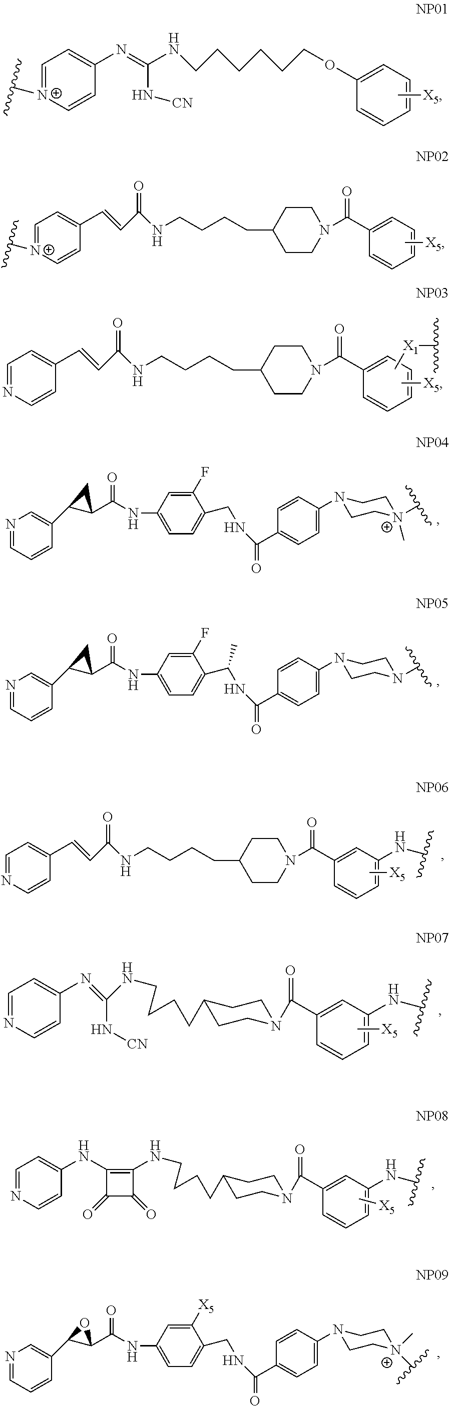
or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁; R₁ is H, C₁˜C₈ alkyl, CH₃CO, NH₂, NHCH₃; [symbol]
is a linkage site that links to L₁ and/or L₂ independently.

13. The antibody drug conjugate according to claim 1, wherein the cytotoxic drug, D₁ and D₂, are independently selected from, a benzodiazepine dimer, having the following formula PB01˜PB30: 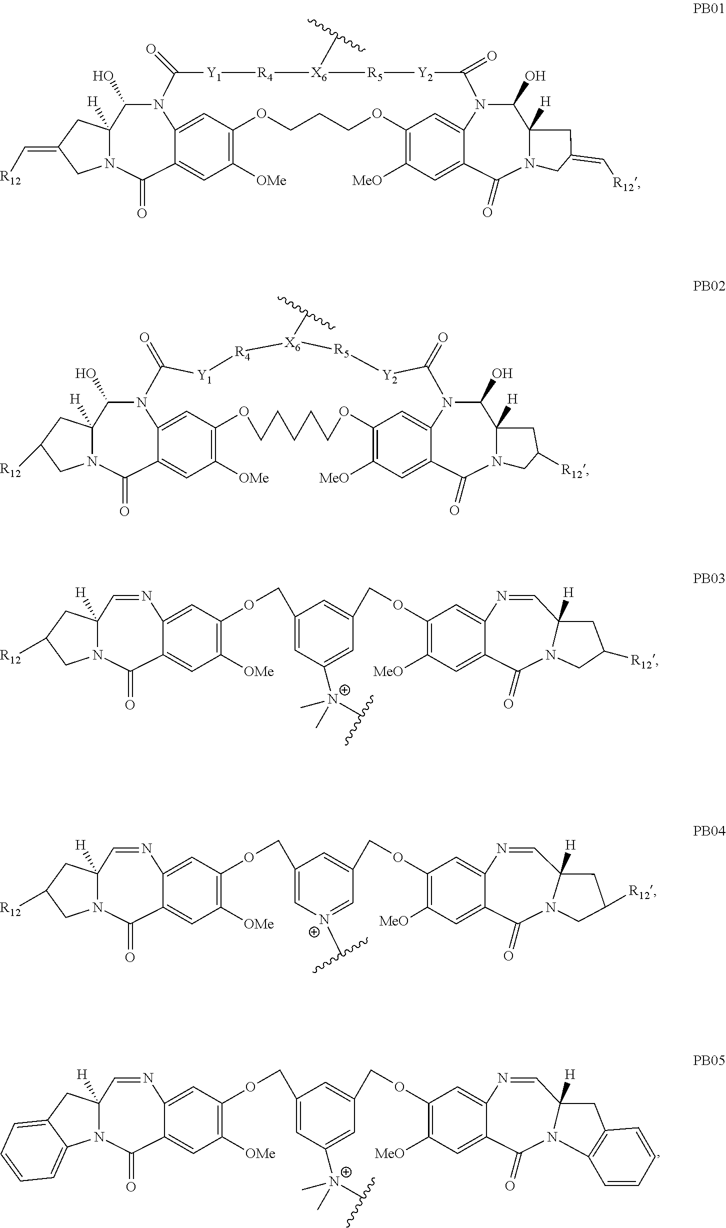 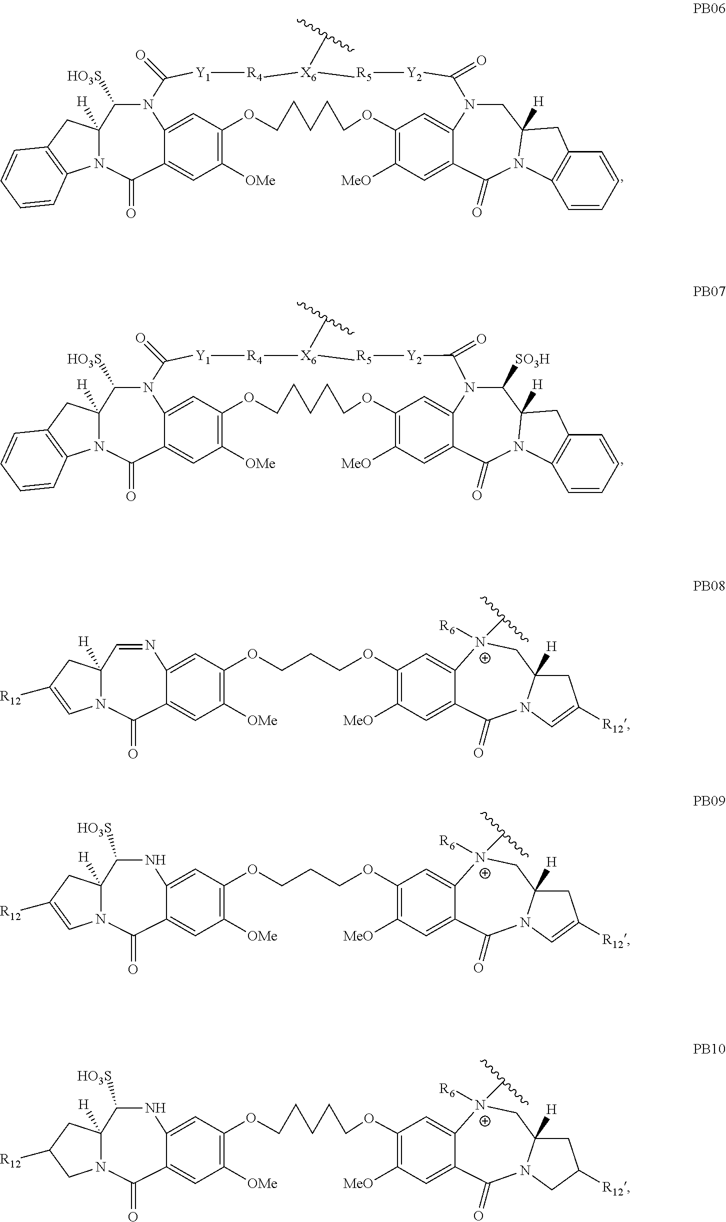 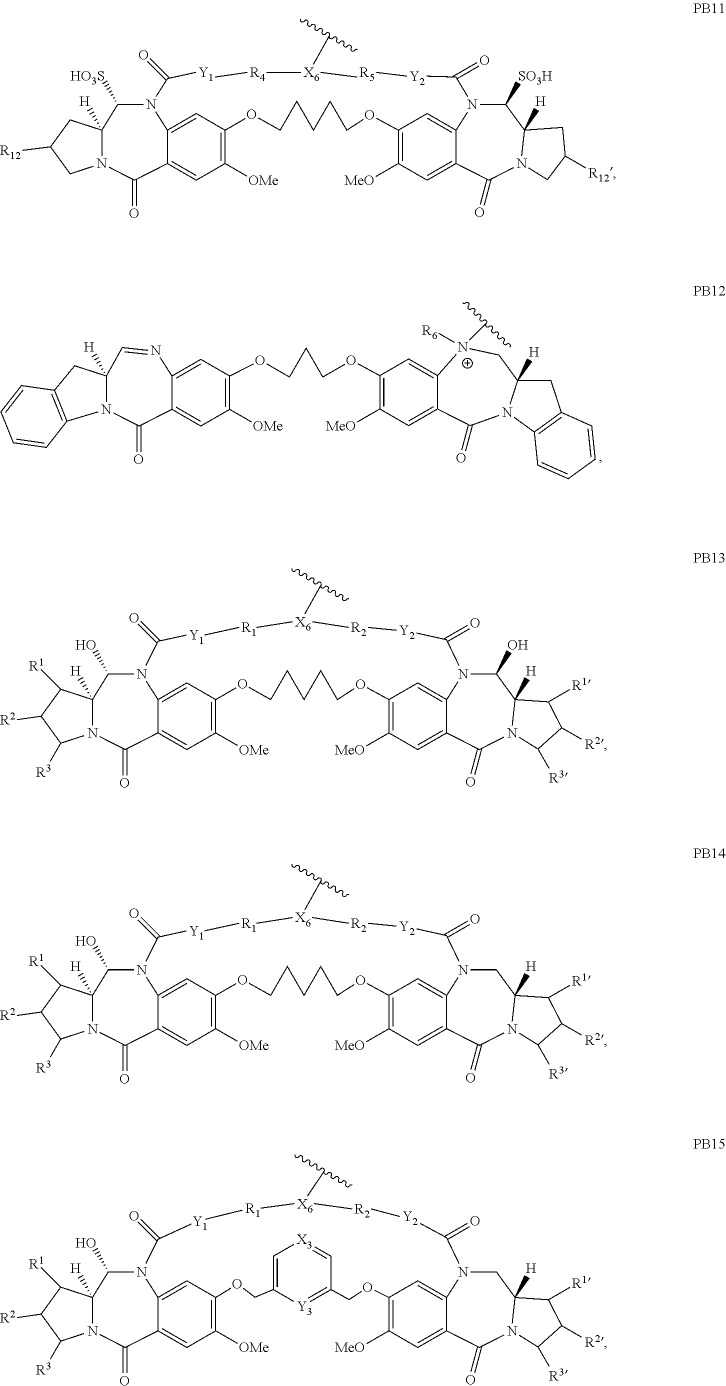 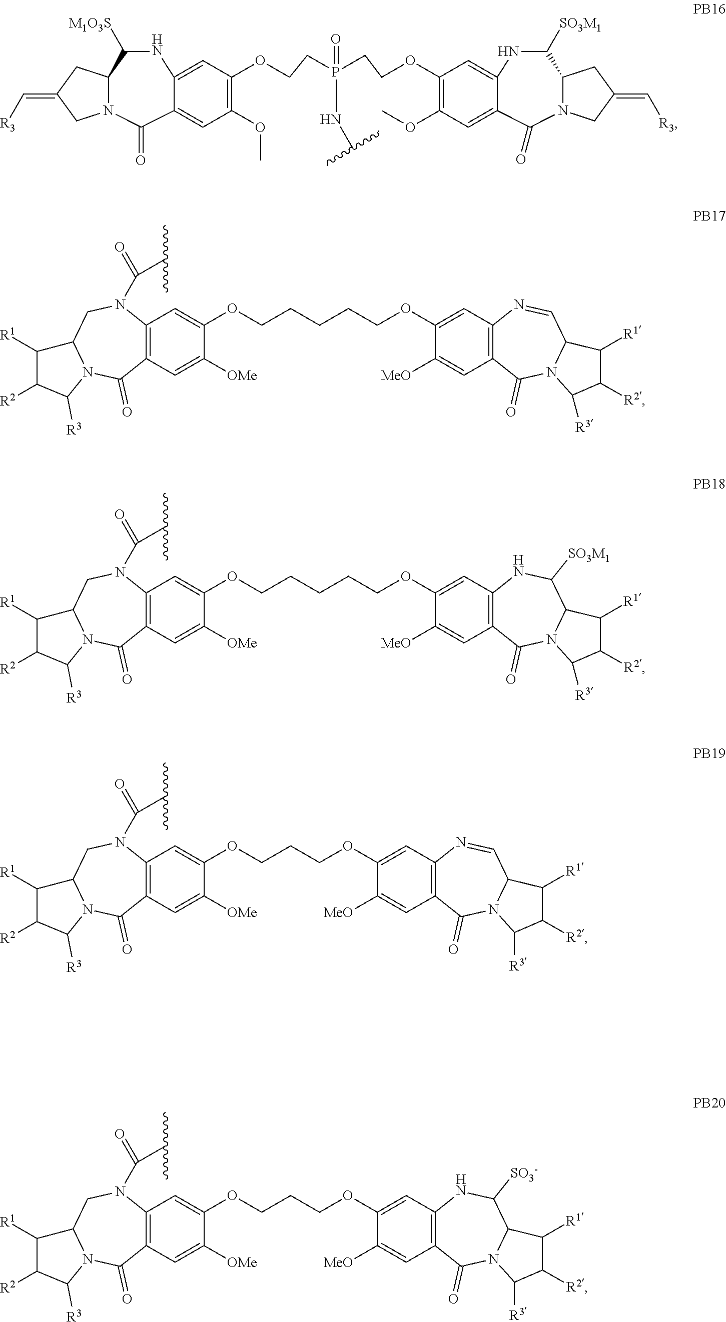 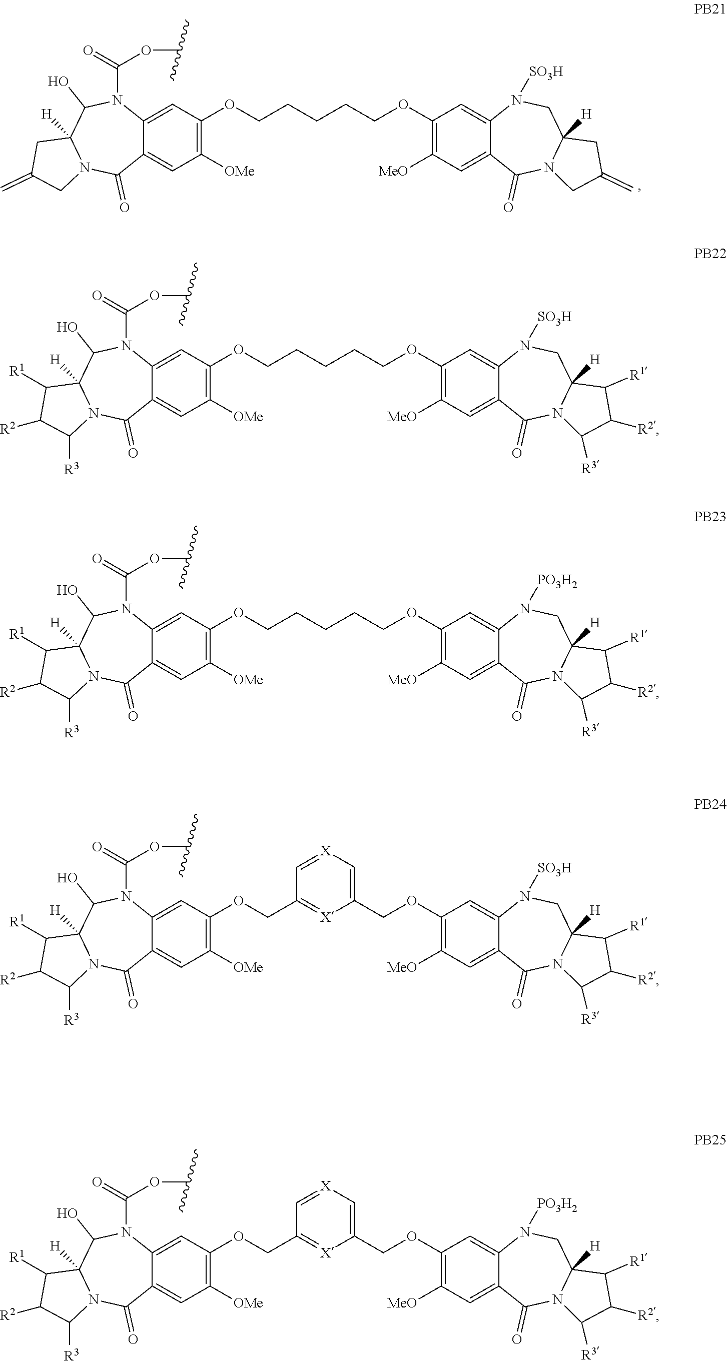 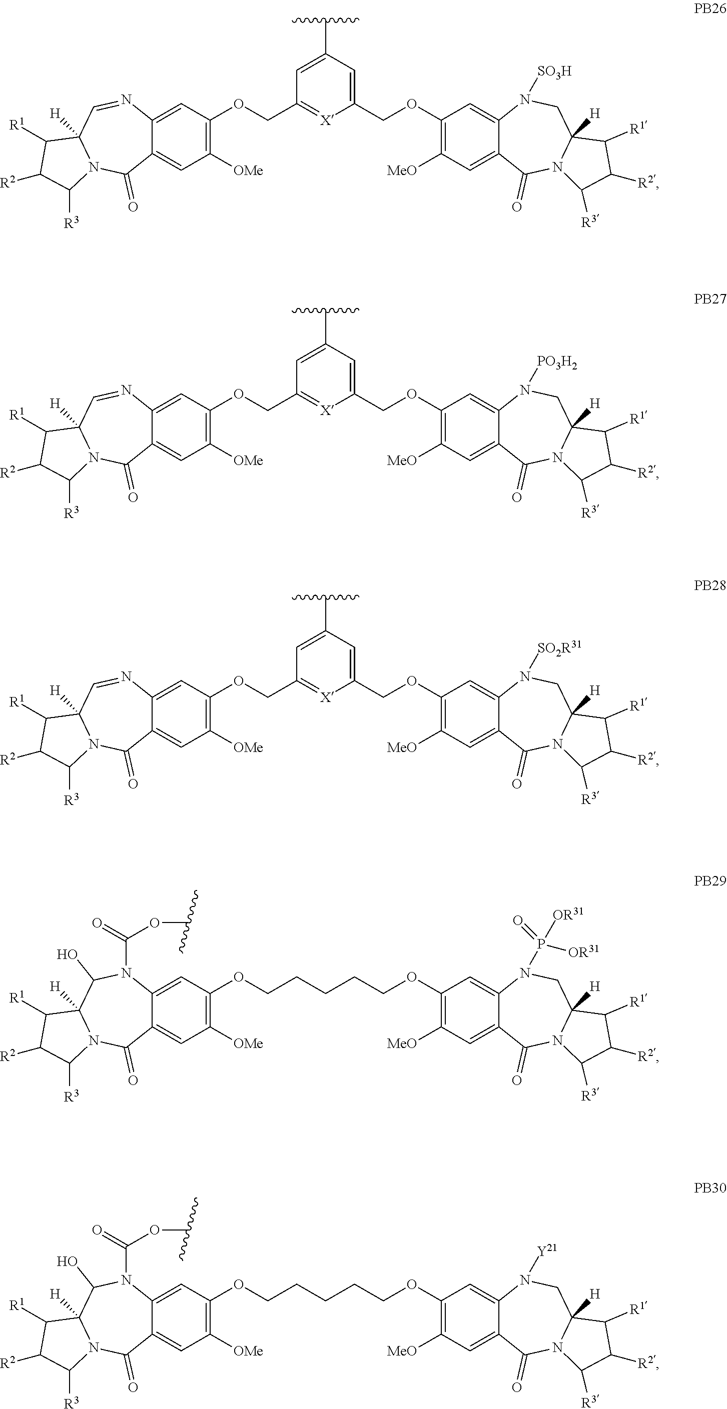
or hydrated salts; or the polymorphic crystalline structures of these compounds; or the optical isomers, racemates, diastereomers or enantiomers; wherein X₁, X₂, Y₁, Y₂, Z₁, Z₂, and n are defined the same above;
R¹, R², R³, R^{1′}, R^{2′}, and R^{3′} are independently H; F; Cl; ═O; ═S; ═CH₂; ═CH—R₁, OH; SH; C₁-C₈ linear or branched alkyl, aryl, alkenyl, heteroaryl, heteroalkyl, alkylcycloalkyl, ester (COOR₅ or —OC(O)R₅), ether (OR₁), amide (CONR₁), carbamate (OCONR₁), amines (NHR₁, NR₁R₂), heterocycloalkyl, or acyloxylamines (—C(O)NHOH, —ONHC(O)R₁); or peptides containing 1-6 natural or unnatural aminoacids, or polyethyleneoxy unit of formula (OCH₂CH₂)ₚ or (OCH₂CH(CH₃))ₚ, wherein p is an integer from 1 to about 100, two Rs: R₁R₂, R₂R₃, R_{1′}R_{2′}, or R_{2′}R_{3′}, can independently form 3˜8-member cyclic ring of C₂˜C₁₀ alkyl, aryl, heteroaryl, heteroalkyl, or alkylcycloalkyl group;
X₃, X, X′ and Y₃ are independently N, O, S, NH, CH₂ or CR1; or one of X₃, X, X′ and Y₃ can be absent, thus the left X₃, X, X′ or Y₃ can be O, S, NH;
wherein R₁, R₂, R₄, and R₅, are C₁-C₈ alkyl, heteroalkyl; C₂-C₈ aminoalkylcarbonyl, alkylaryloxyl, alkylarylamino, alkylarylthiol; or 1-6 the same or different sequence of aminao acid/peptides (Aa) r, r=1, 2, 3, 4, 5, or 6;
wherein R₁₂ and R₁₂′ are independently H, OH, NH₂, NH(CH₃), NHNH₂, COOH, SH, OZ₃, SZ₃, F, Cl, or C₁-C₈ linear or branched alkyl, C₃-C₈ aryl, heteroaryl, heteroalkyl, alkylcycloalkyl, acyloxylamines;
X₆ is CH, N, P(O)NH, P(O)NR₁, CHC(O)NH, C₃-C₈ aryl, heteroaryl, alkylcycloalkyl, acyloxyl, alkylaryl, alkylaryloxyl, alkylarylamino, or an Aa (amino acid);
Y₂₁ is Ms (mesyl), Ts (tosyl) or Tf (trifyl), SO₃H, P(O)(OH)₂, CH₂ (O)P(O)(OH)₂, glycoside;
R₃₁ is H, C₁-C₈ alkyl or Ar, CF₃; [symbol]
is a linkage site that links to L₁ and/or L₂ independently.

14. The antibody drug conjugate according to claim 1, wherein the cytotoxic drug, D₁ and D₂, are independently selected from, a doucarmycin analog, having the following formula CC01, CC02, CC03, CC04, CC05, CC06 and CC07: 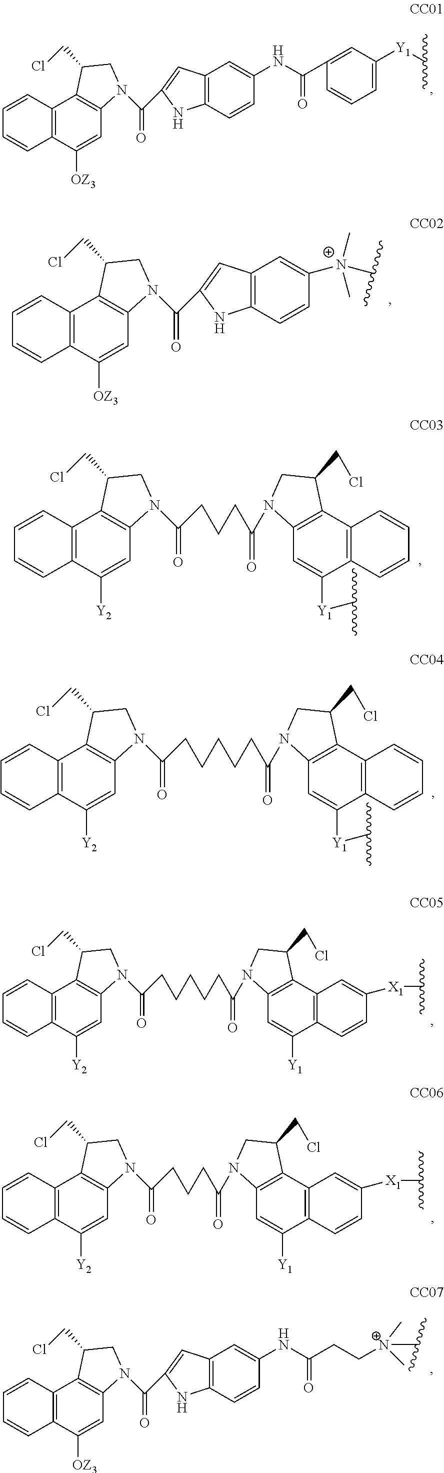
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHANH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”;
Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, and R₃, are C₁-C₈alkyl, heteroalkyl; C₂-C₈ aminoalkylcarbonyl; or 1-4 the same or different sequence of aminao acid/peptides (Aa) r, r=1, 2, 3, 4; M₁, and M2 are pharmaceutical salt selected from Na, K, Ca, NH₄, NH₃CH₂CH₂OH or Zn.

15. The antibody drug conjugate according to claim 1, wherein the cytotoxic drug, D₁ and D₂, are independently selected from, an amatoxin, having the following formula Am01, Am02, and Am03: 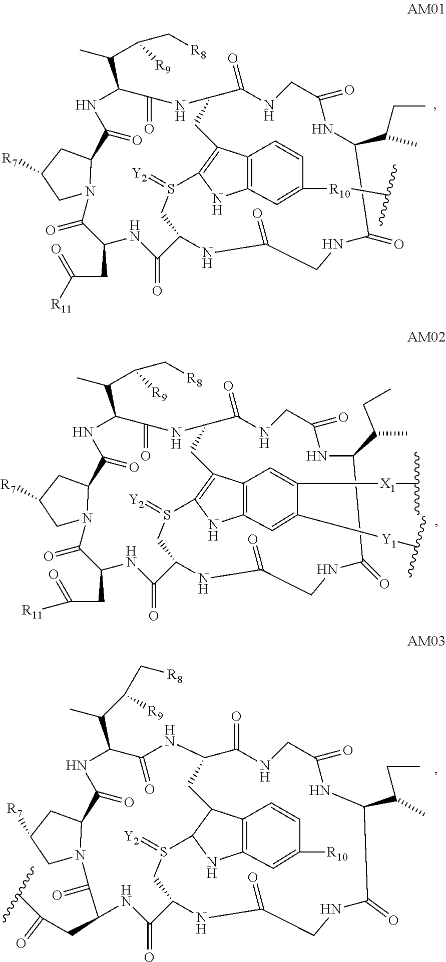
or an isotope of one or more chemical elements, or pharmaceutically acceptable salts, hydrates, or hydrated salts; or the polymorphic crystalline structures of these compounds; or the optical isomers, racemates, diastereomers or enantiomers;
wherein X₁, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, CHNH, CH₂O, C(O)NH—NHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa), COOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₂, O(CH₂CH₂O)ₚCH₂CH₂—COOH, NH(CH₂CH₂O)ₚCH₂CH₂COOH, NH—Ar—COOH, NH—Ar—NH₂, O(CH₂CH₂O)ₚCH₂CH₂—NHSO₃H, NH(CH₂CH₂O)ₚCH₂CH₂NHSO₃H, R₁—NHSO₃H, NH—R₁—NHSO₃H, O(CH₂CH₂O)ₚ—CH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂—CH₂NHPO₃H₂, wherein (Aa) r is r=1, 2, 3 or 4 aminoacids; n and m₁ are independently 1˜20; p is 1-100; R₁, R₂ and Ar, are the same defined; “ [symbol] ” is a linkage site that links to L₁ and/or L₂ independently.

16. The antibody drug conjugate according to claim 1, wherein the cytotoxic drug, D1 and D2, are independently selected from, a protein kinase inhibitor, having the following formula PK01˜PK40: 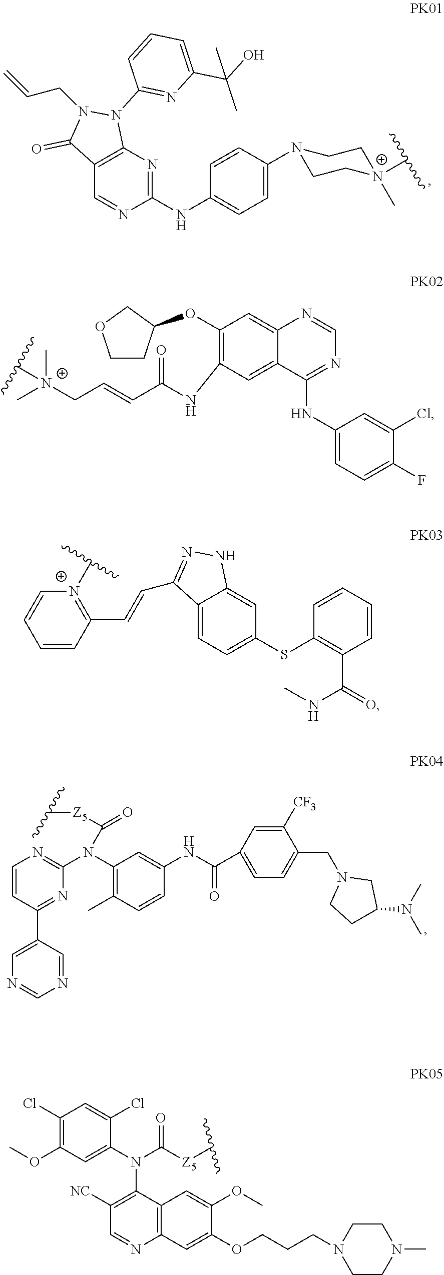 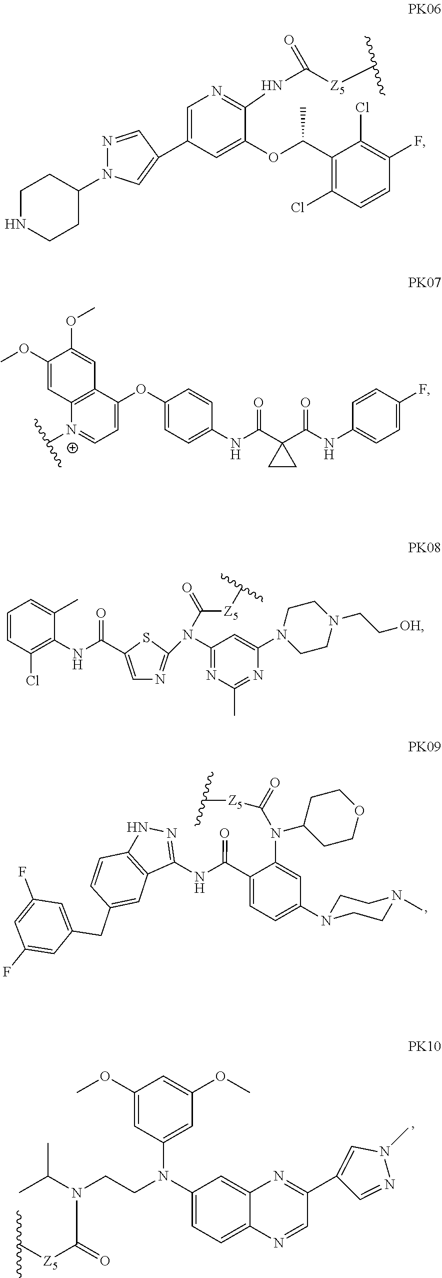 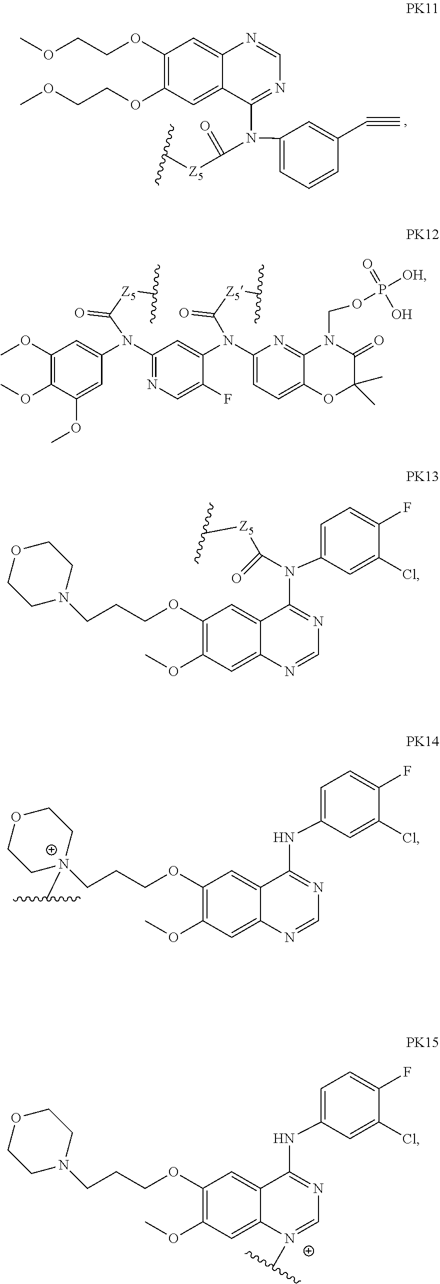 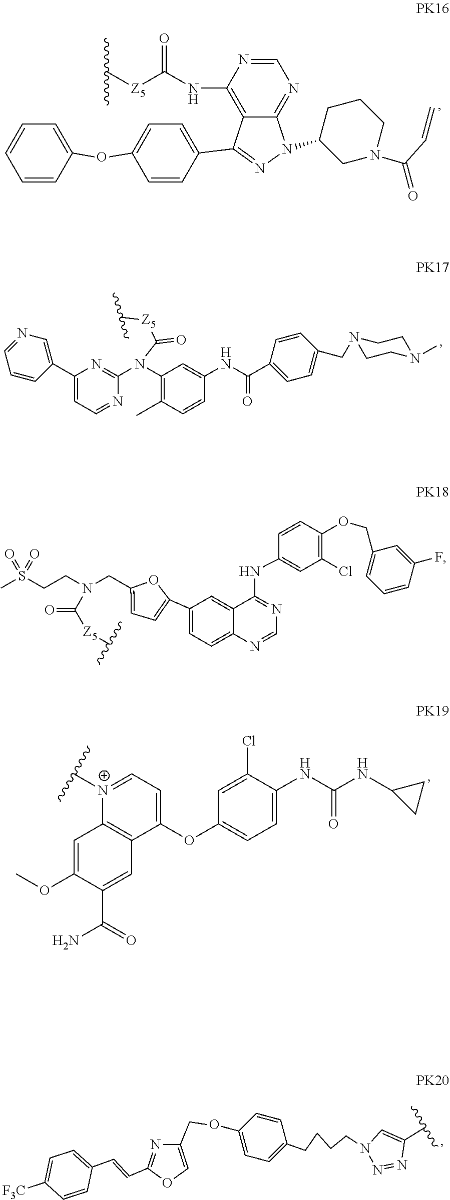 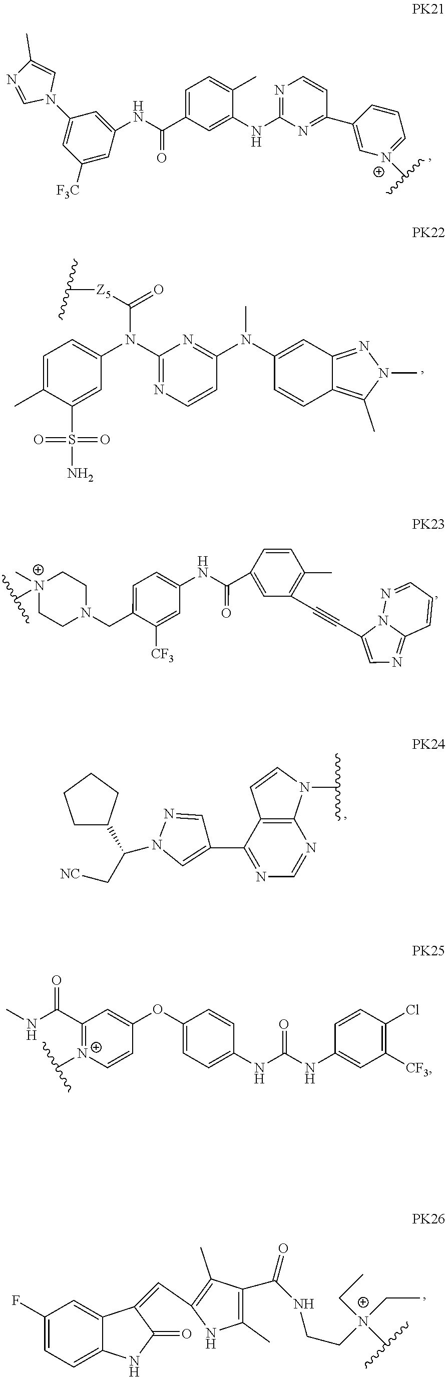 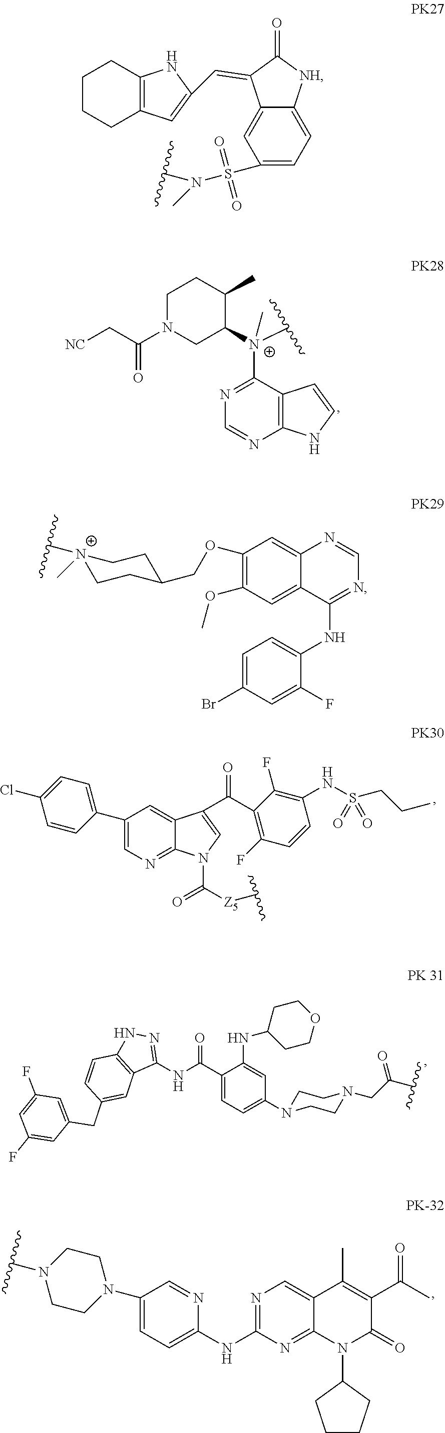 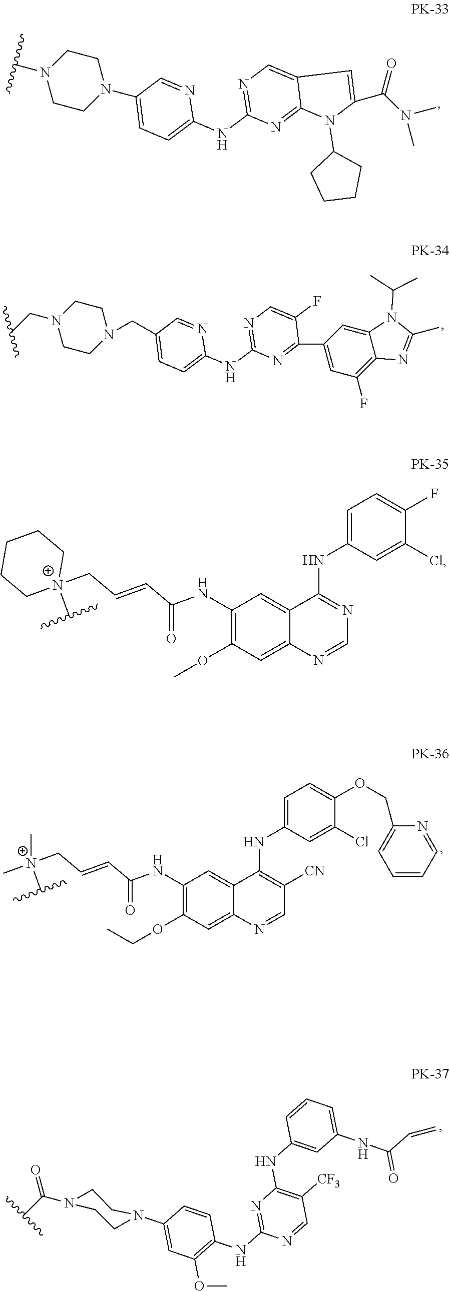 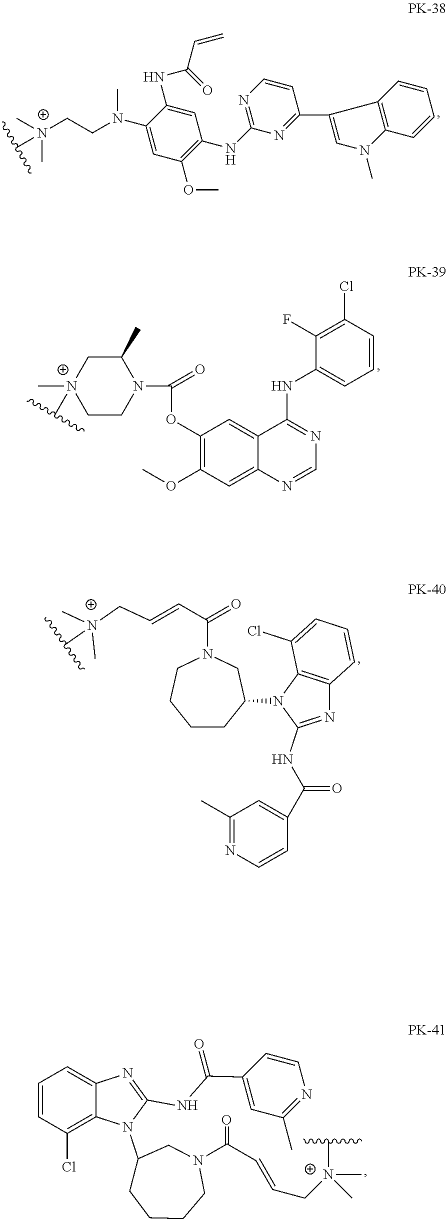 [SMILES: *[N+](C)(C)CCN(C)c1cc(OC)c(Nc2nccc(-c3cn(C)c4ccccc34)n2)cc1NC(=O)C=C ; *[N+]1(C)CCN(C(=O)Oc2cc3c(Nc4cccc(Cl)c4F)ncnc3cc2OC)[C@H](C)C1 ; *[N+](C)(C)C/C=C/C(=O)N1CCCC[C@@H](n2c(NC(=O)c3ccnc(C)c3)nc3cccc(Cl)c32)C1 ; *[N+](C)(C)C/C=C/C(=O)N1CCCCC(n2c(NC(=O)c3ccnc(C)c3)nc3cccc(Cl)c32)C1]
or hydrated salts; or the polymorphic crystalline structures of these compounds; or the optical isomers, racemates, diastereomers or enantiomers;
wherein Z₅ and Zs' are independently selected from O, NH, NHNH, NR₅, S, C(O)O, C(O)NH, OC(O)NH, OC(O)O, NHC(O)O, NHC(O)NH, NHC(O) S, OC(O)N(R₁), N(R₁) C(O)N(R₂), C(O)NHNHC(O) and C(O)NR₁; [symbol] is a linkage site that links to L₁ and/or L₂ independently.

17. The antibody drug conjugate according to claim 1, wherein the cytotoxic drug, D₁ and D₂, are independently selected from, an immunotoxin, selected from Diphtheria toxin (DT), Cholera toxin (CT), Trichosanthin (TCS), Dianthin, Pseudomonas exotoxin A (ETA), Erythrogenic toxins, Diphtheria toxin, AB toxins, Type III exotoxins, proaerolysin, and topsalysin.

18. The antibody drug conjugate according to claim 1, wherein the cytotoxic drug, D₁ and D2, are independently selected from, one, two or more DNA, RNA, mRNA, small interfering RNA (siRNA), microRNA (miRNA), and PIWI interacting RNAs (piRNA), having the following formula SI01 and SI02: 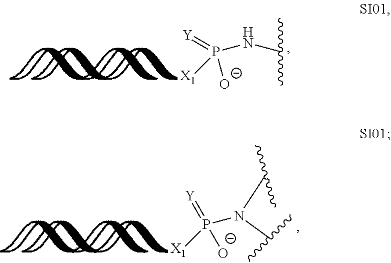
wherein “ [symbol] ” is the site to link the side chain linker of the present patent; [symbol] is single or double strands of DNA, RNA, mRNA, siRNA, miRNA, or piRNA; X₁, and Y are independently O, NH, NHNH, NR₁, S, C(O)O, C(O)NH, OC(O)NH, OC(O)O, NHC(O)NH, NHC(O) S, OC(O)N(R₁), N(R₁)C(O)N(R₁), CH₂, C(O)NHNHC(O) and C(O)NR₁, [symbol] is a linkage site.

19. The antibody drug conjugate according to claim 1, wherein the cytotoxic drug, D₁ and D₂, are independently selected from a MEK inhibitor having the following formula: 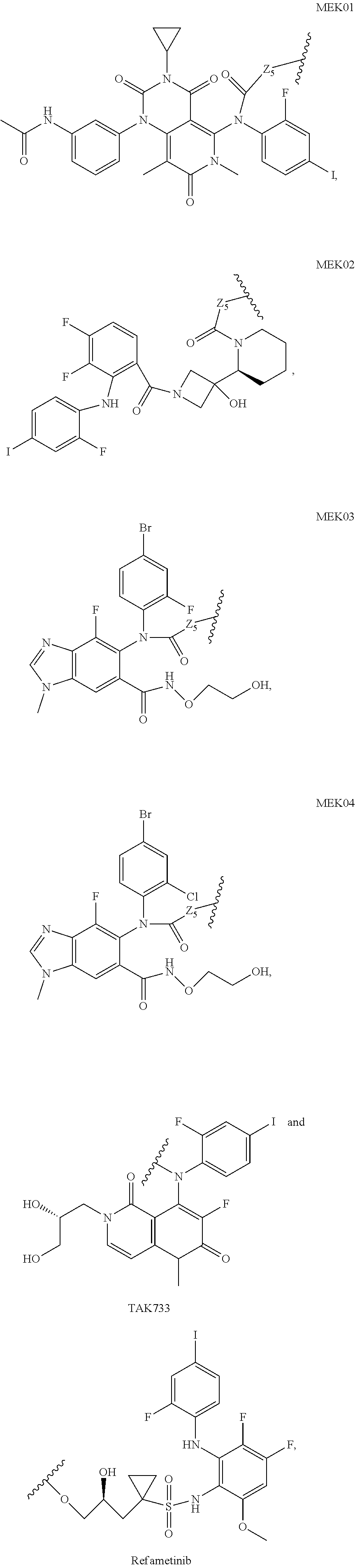
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein Zs is selected from O, NH, NHNH, NR₅, S, C(O)O, C(O)NH, OC(O)NH, OC(O)O, NHC(O)O, NHC(O)NH, NHC(O) S, OC(O)N(R₁), N(R₁)C(O)N(R₂), C(O)NHNHC(O) and C(O)NR₁, [symbol] is a linkage site.

20. The antibody drug conjugate according to claim 1, wherein the cytotoxic drug, D₁ and D₂, are independently selected from, a Poly (ADP-ribose) polymerase (PARP) inhibitor, having the following formula: 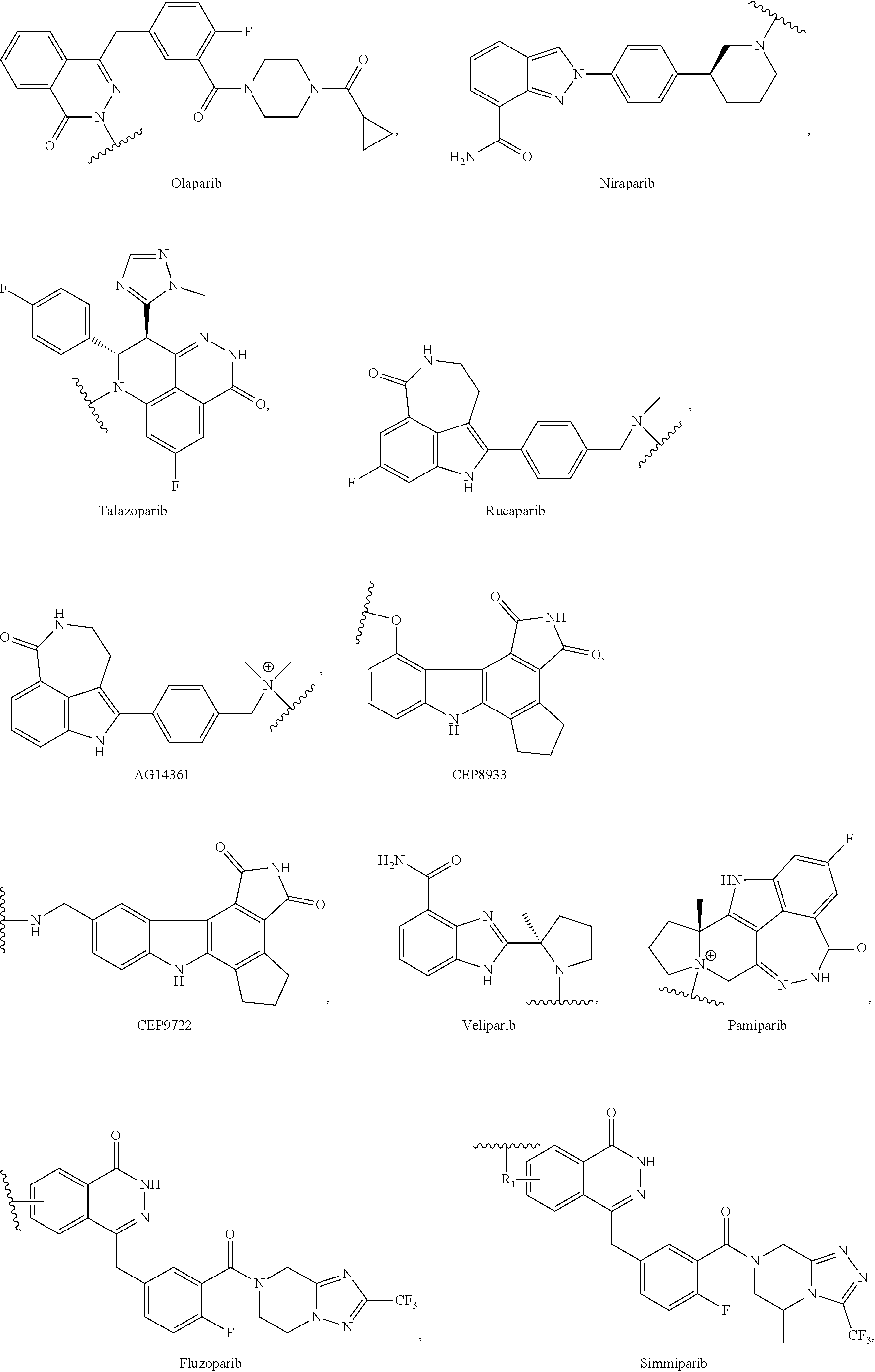 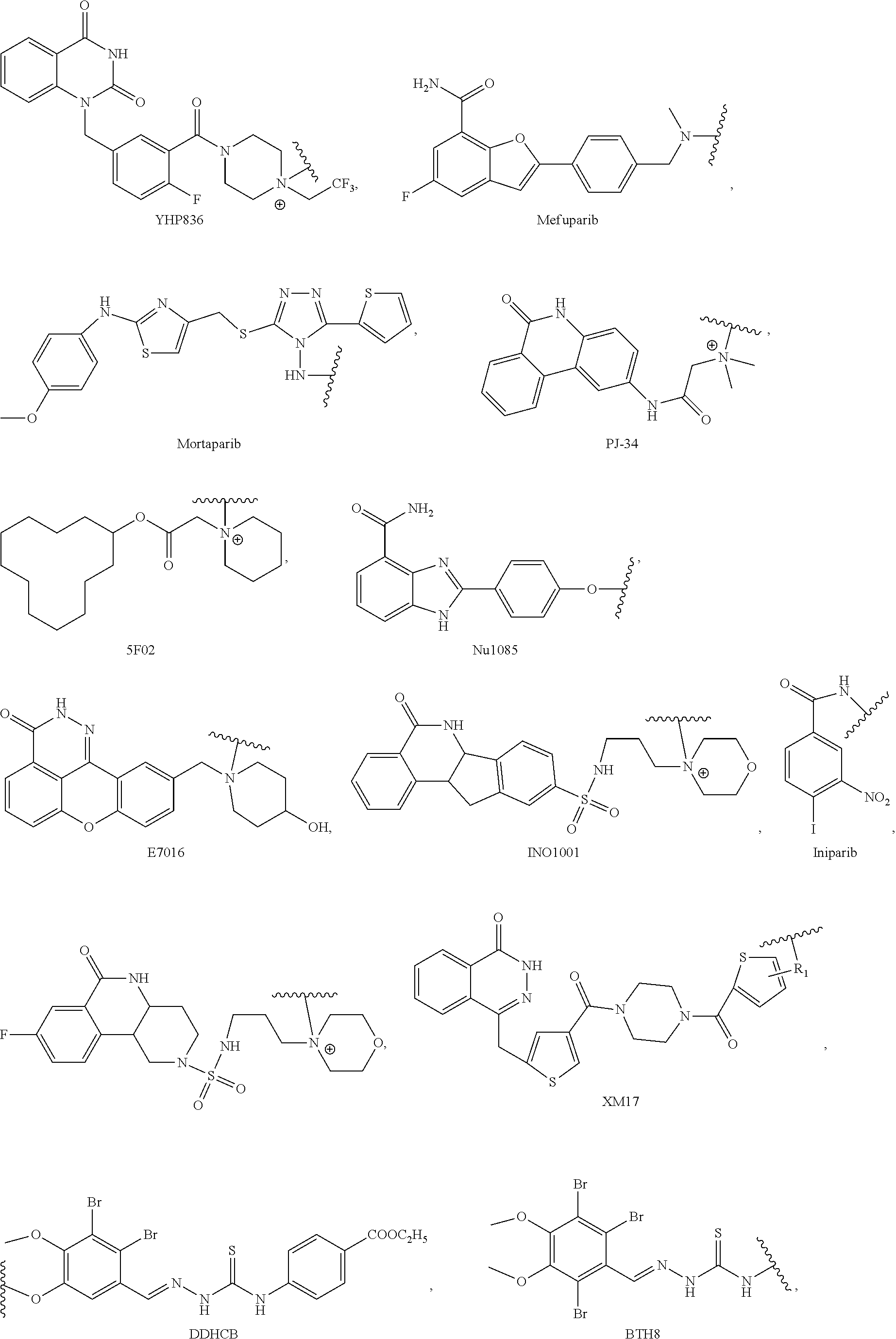 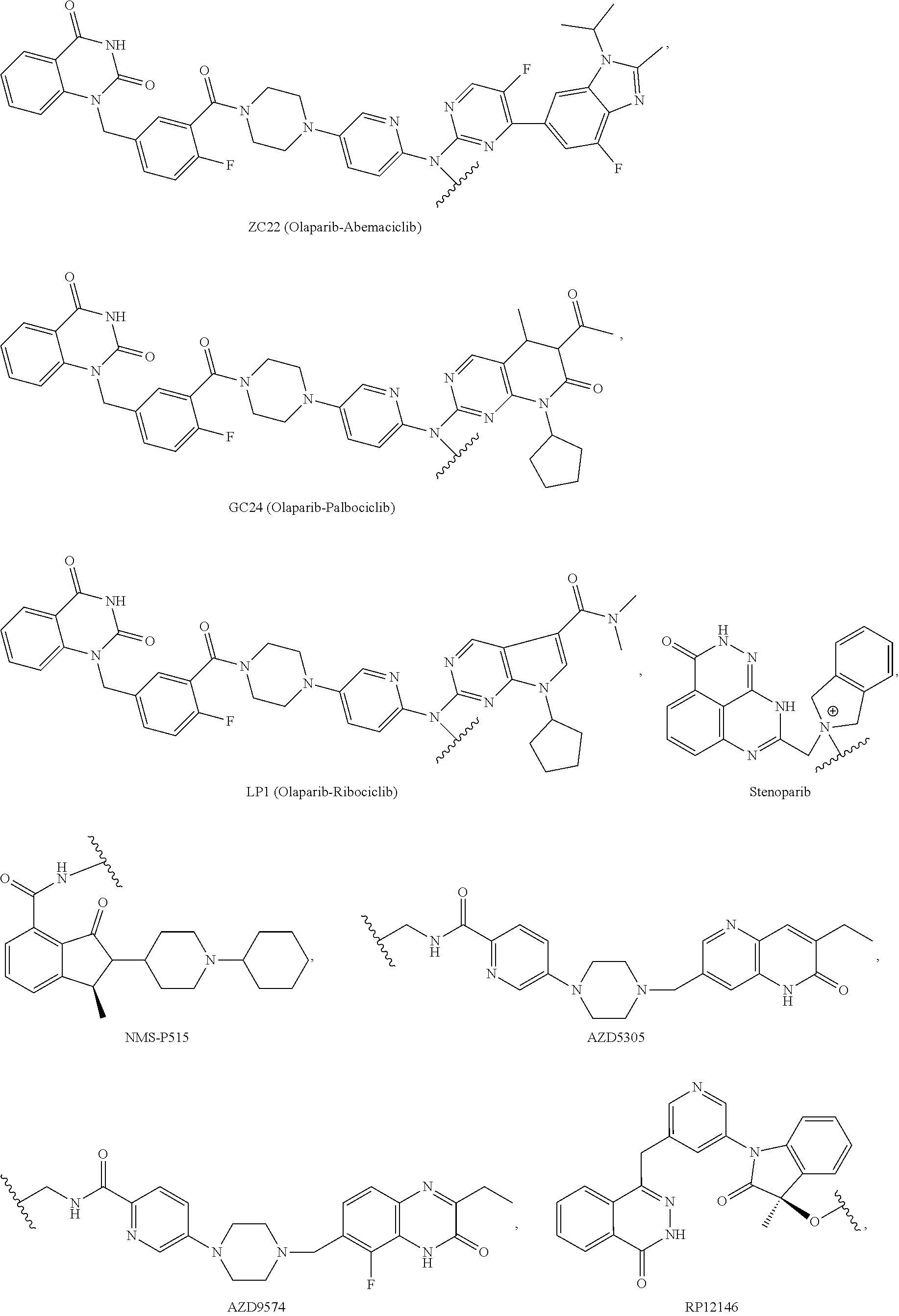 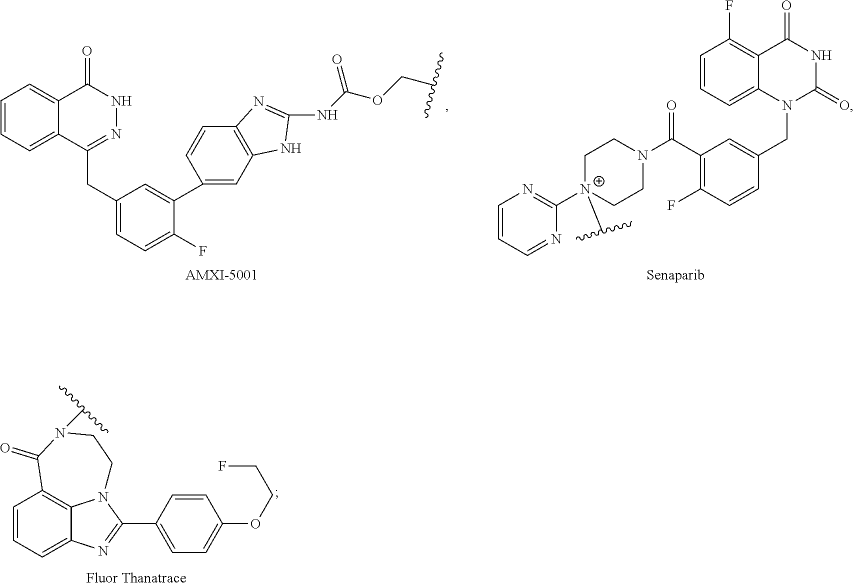 [SMILES: *COC(=O)Nc1nc2ccc(-c3cc(Cc4n[nH]c(=O)c5ccccc45)ccc3F)cc2[nH]1 ; *[N+]1(c2ncccn2)CCN(C(=O)c2cc(Cn3c(=O)[nH]c(=O)c4c(F)cccc43)ccc2F)CC1 ; *N1CCn2c(-c3ccc(OCCF)cc3)nc3cccc(c32)C1=O]
or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the linkage site.

21. The antibody drug conjugate according to claim 1, wherein the cytotoxic drug, D₁ and D₂, are independently selected from, an Inhibitor of Bromodomain-containing proteins, having the following formula: 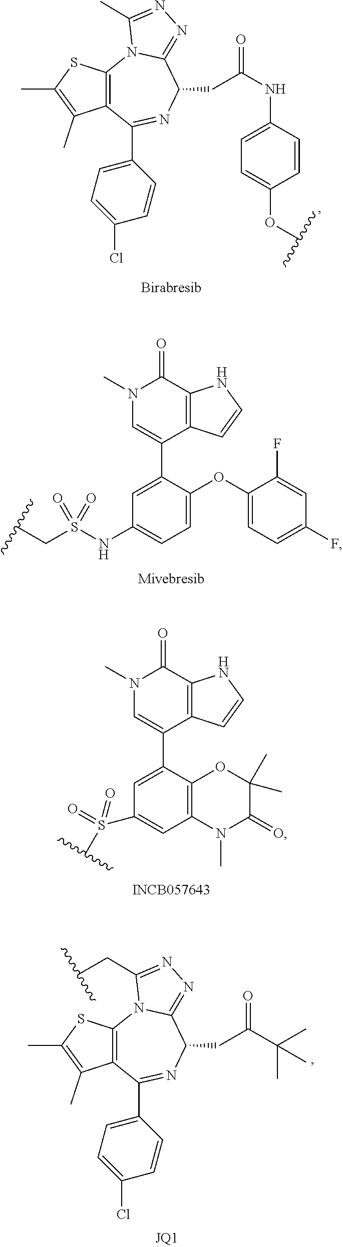 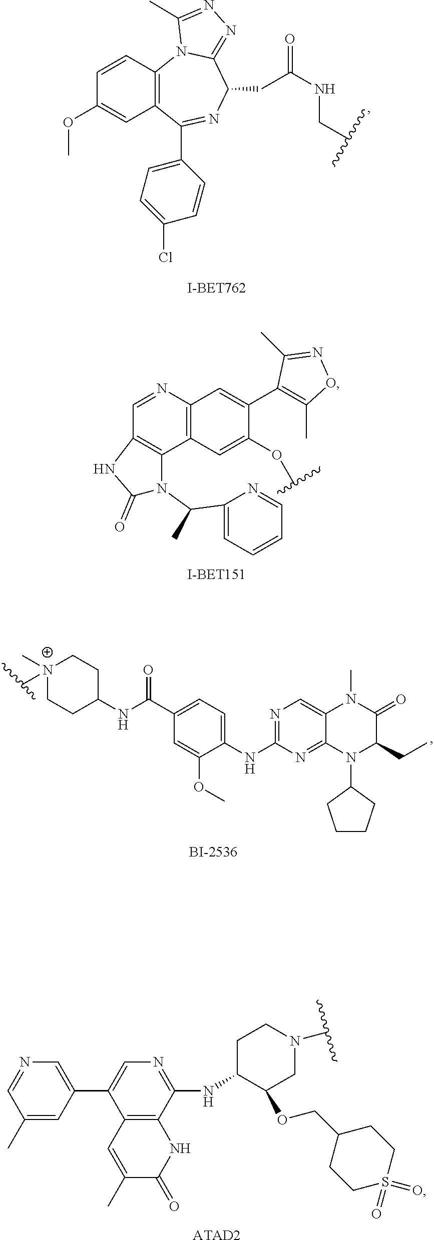 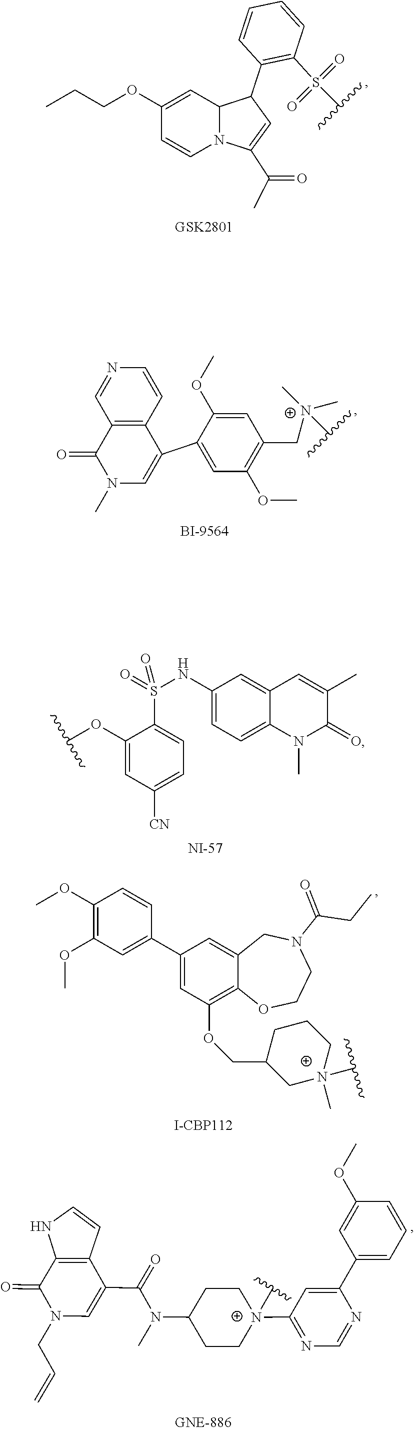 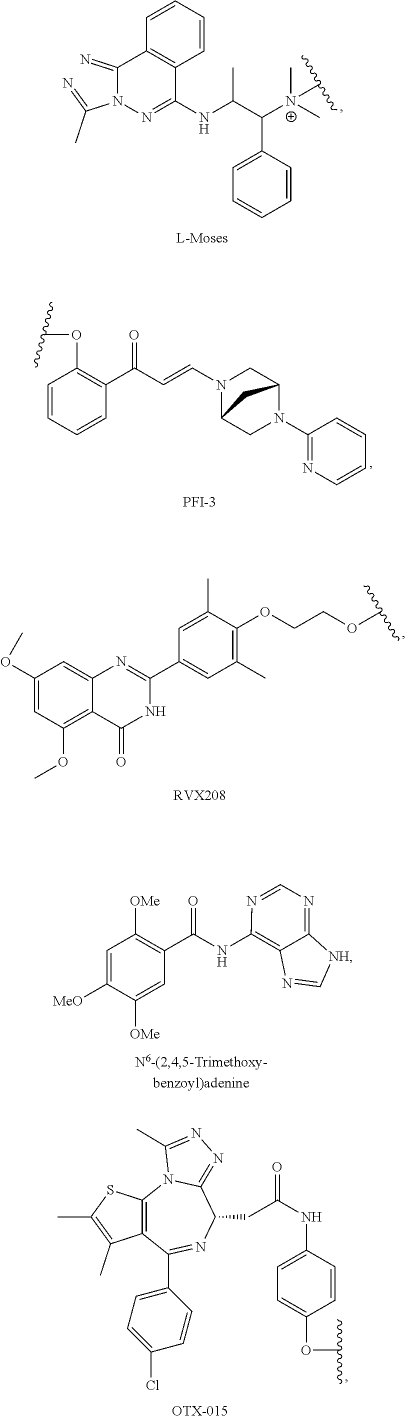 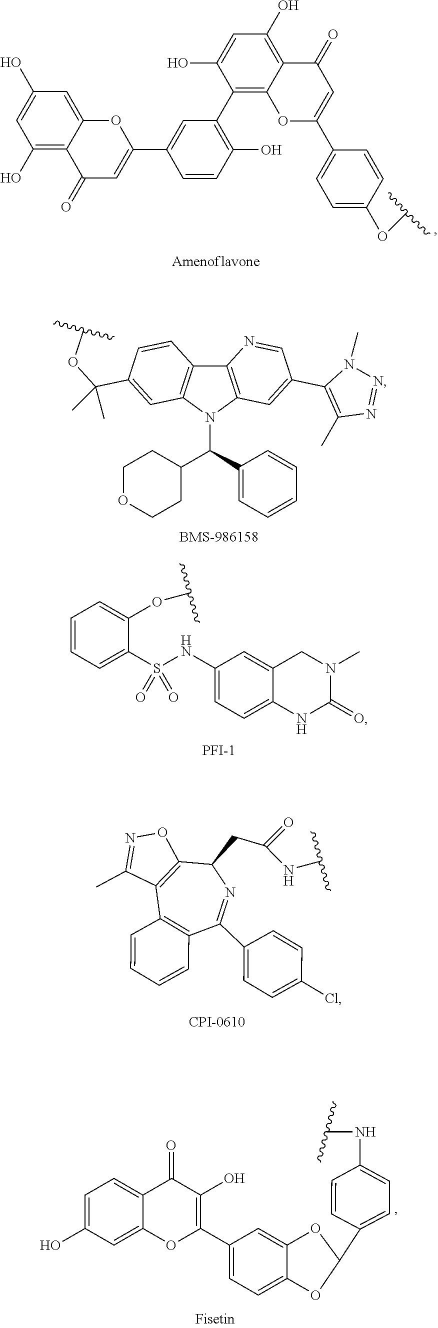 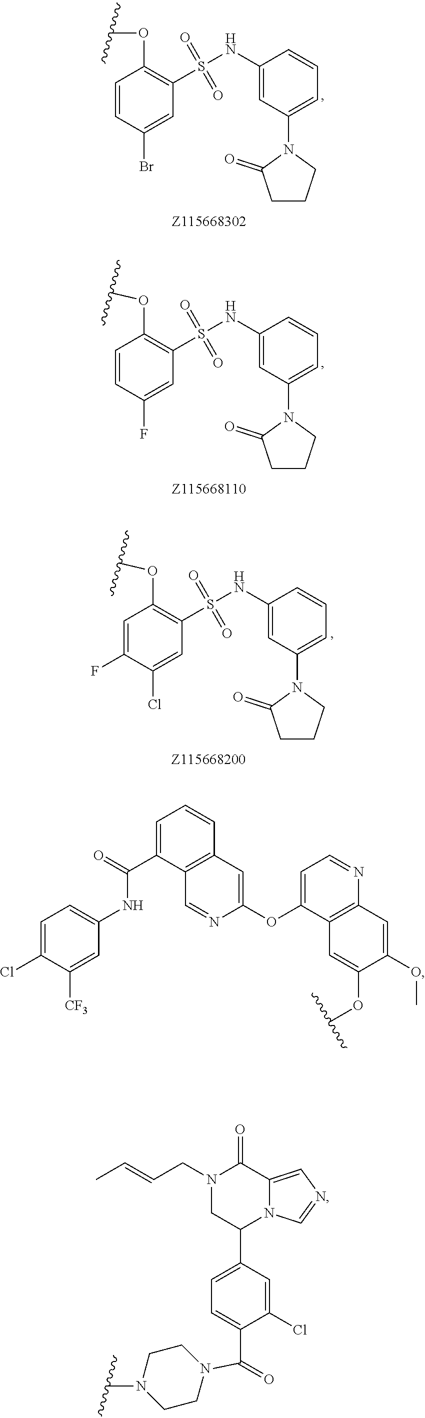 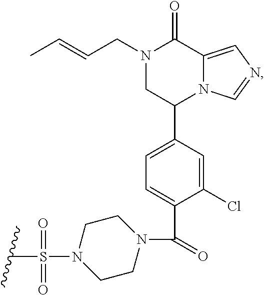 [SMILES: *S(=O)(=O)N1CCN(C(=O)c2ccc(C3CN(C/C=C/C)C(=O)c4cncn43)cc2Cl)CC1]
or hydrated salts; or the polymorphic crystalline structures of these compounds; or the optical isomers, racemates, diastereomers or enantiomers; [symbol] is a linkage site that links to L₁ and/or L₂ independently.

22. The antibody drug conjugate according to claim 1, wherein the cytotoxic drug, D₁ and D₂, are independently selected from, an Inhibitor of Cyclin-dependent kinases (CDKs), having the following formula: 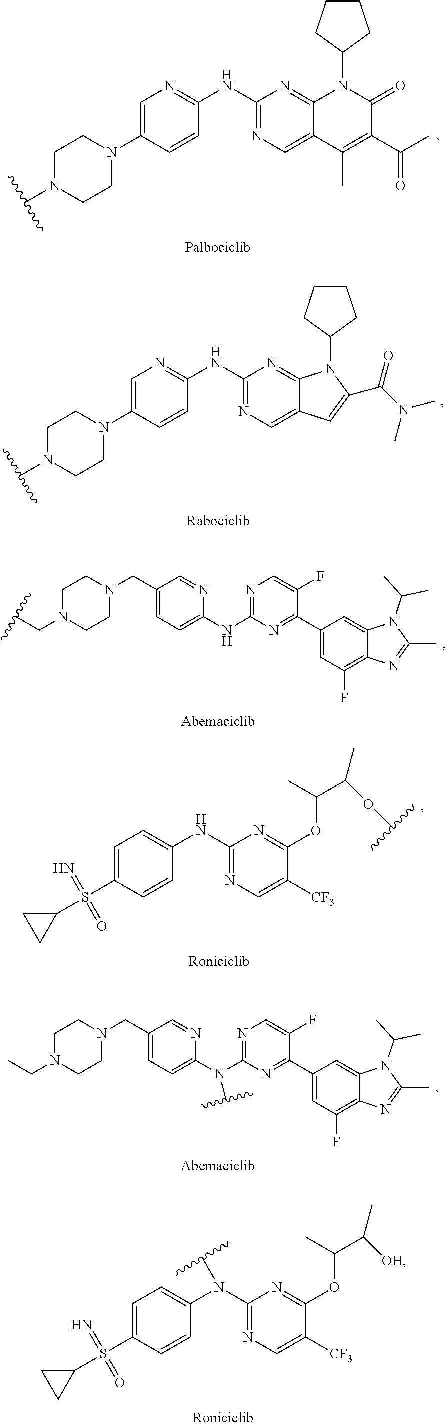 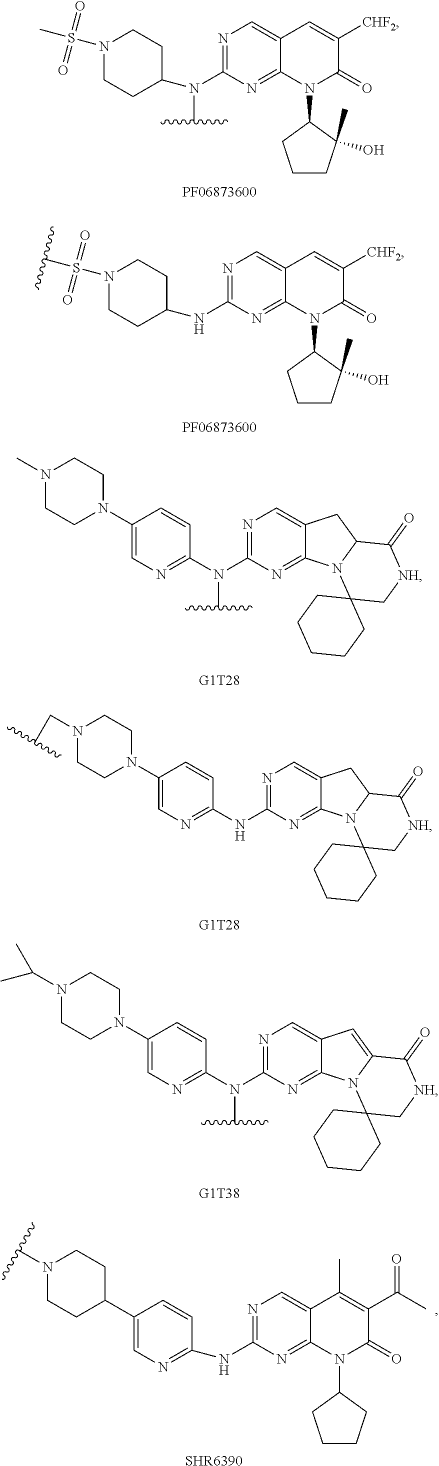 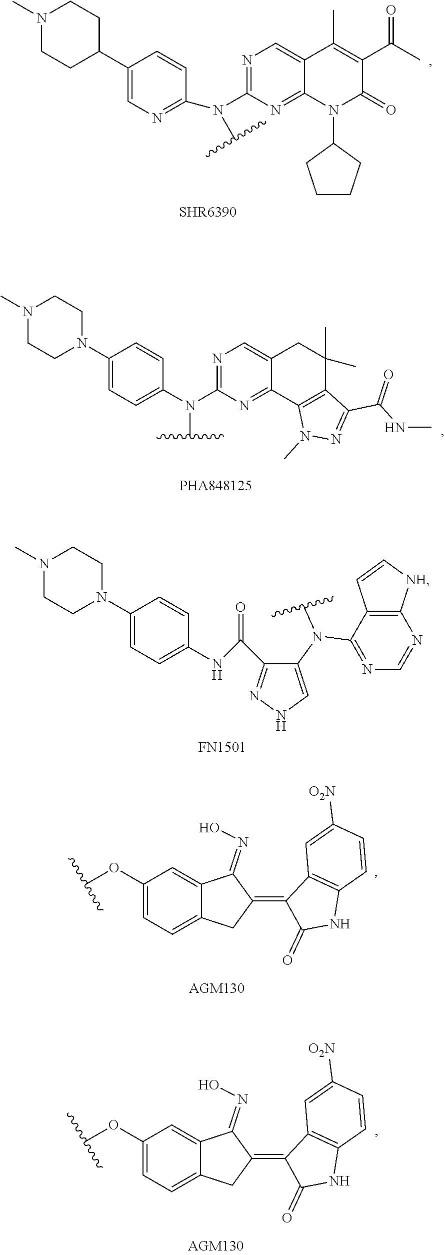 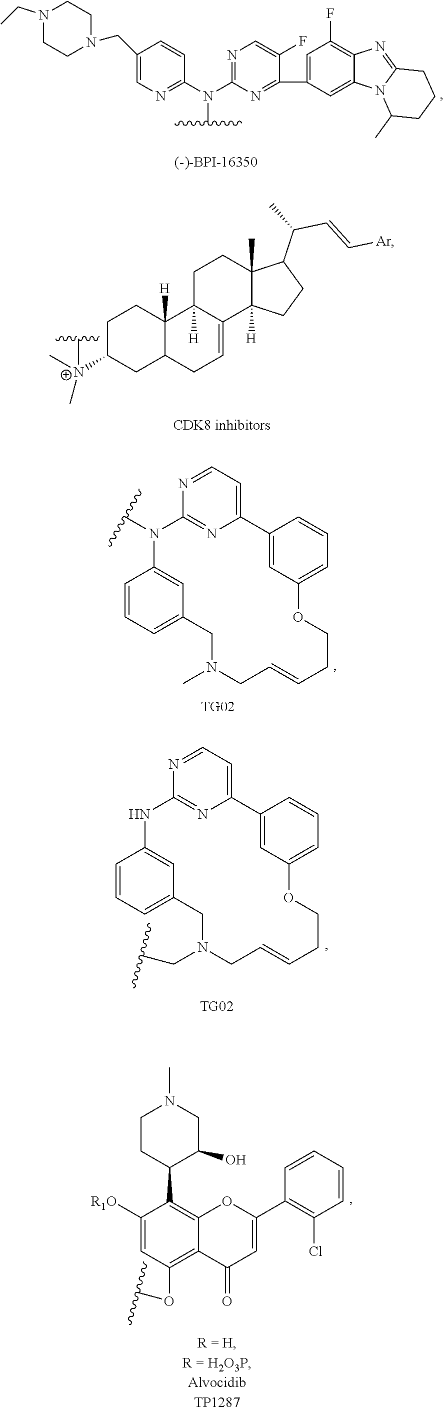 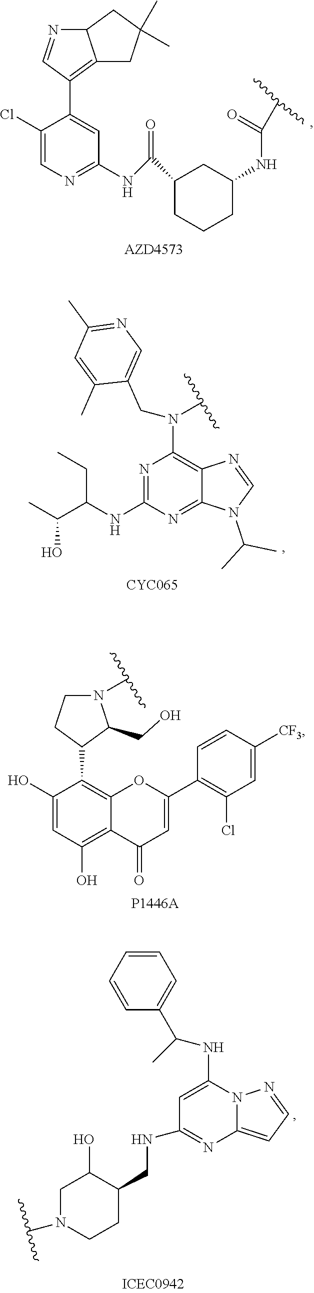 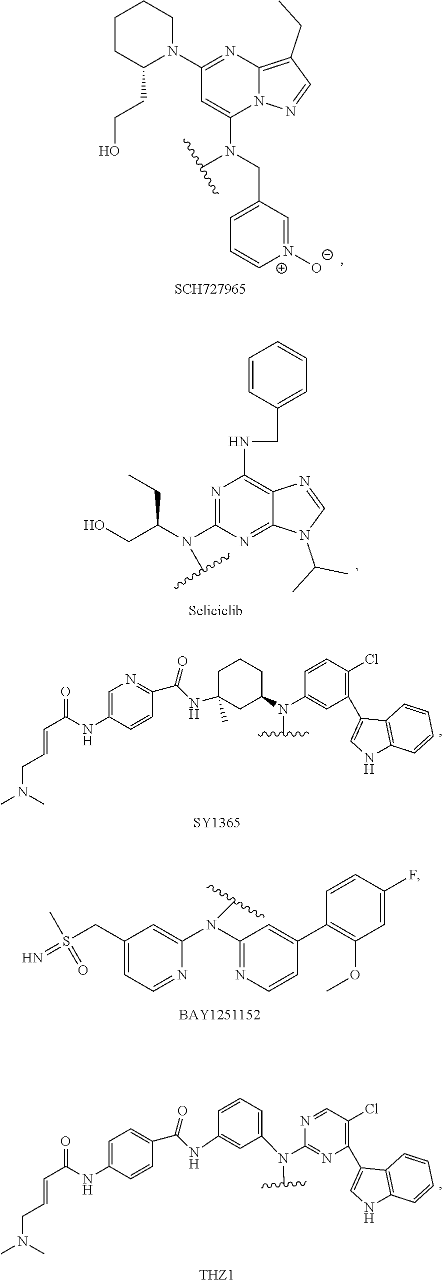 [SMILES: *N(Cc1ccc[n+]([O-])c1)c1cc(N2CCCC[C@H]2CCO)nc2c(CC)cnn12 ; *N(c1nc(NCc2ccccc2)c2ncn(C(C)C)c2n1)[C@H](CC)CO ; *N(c1ccc(Cl)c(-c2c[nH]c3ccccc23)c1)[C@@H]1CCC[C@](C)(NC(=O)c2ccc(NC(=O)/C=C/CN(C)C)cn2)C1 ; *N(c1cc(CS(C)(=N)=O)ccn1)c1cc(-c2ccc(F)cc2OC)ccn1 ; *N(c1cccc(NC(=O)c2ccc(NC(=O)/C=C/CN(C)C)cc2)c1)c1ncc(Cl)c(-c2c[nH]c3ccccc23)n1] 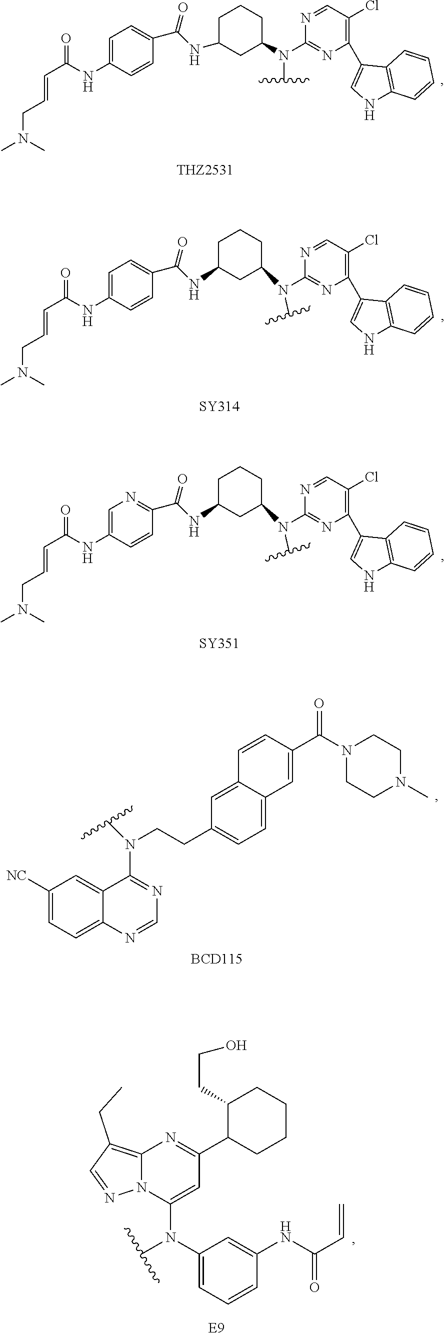 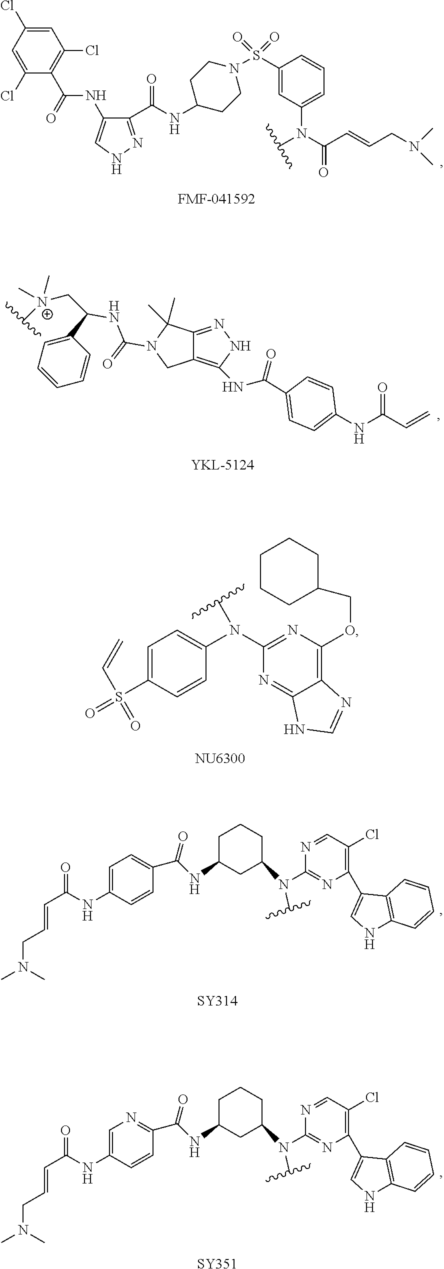 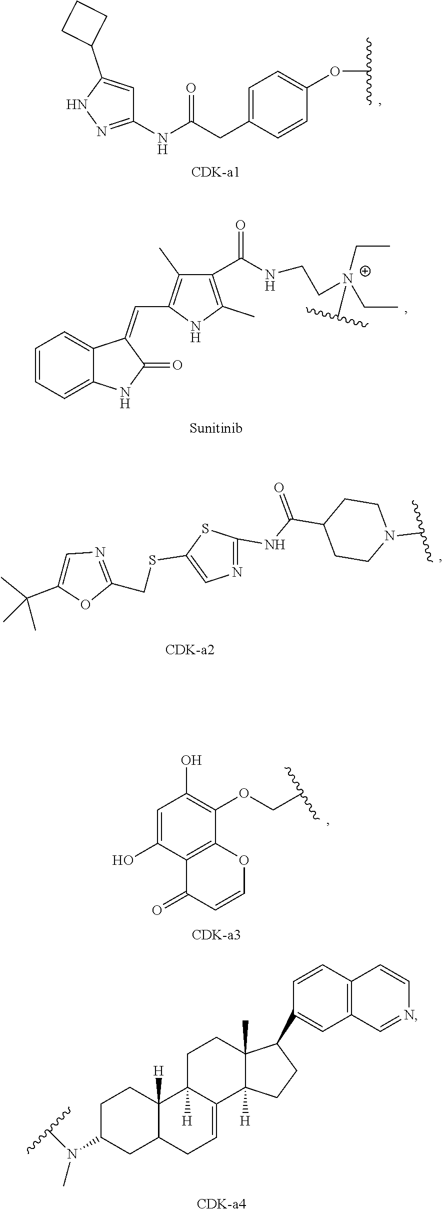 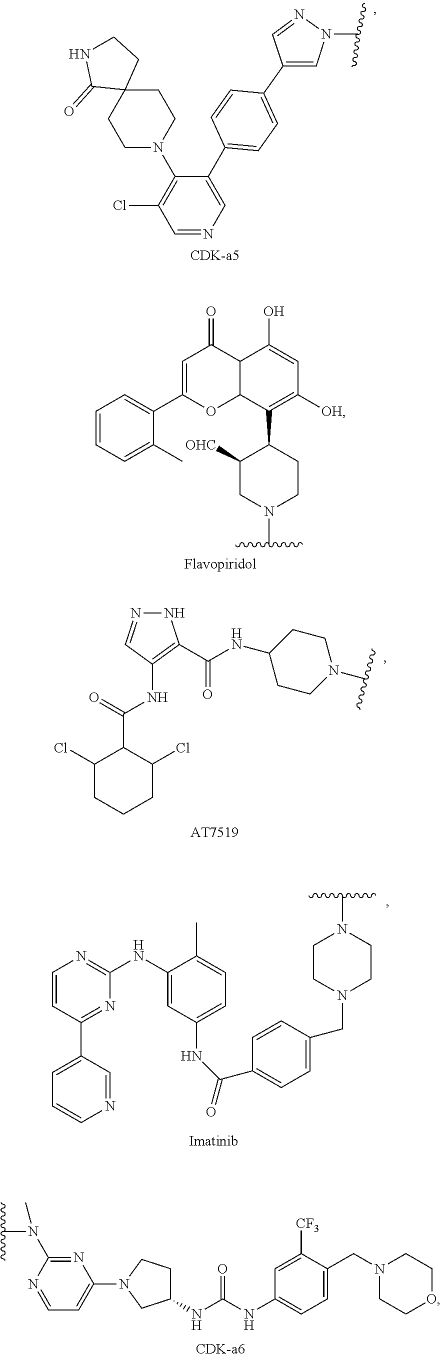 [SMILES: *n1cc(-c2ccc(-c3cncc(Cl)c3N3CCC4(CCNC4=O)CC3)cc2)cn1 ; *N1CC[C@H](C2=C(O)C=C(O)C3C(=O)C=C(c4ccccc4C)OC23)[C@H](C=O)C1 ; *N1CCC(NC(=O)c2[nH]ncc2NC(=O)C2C(Cl)CCCC2Cl)CC1 ; *N1CCN(Cc2ccc(C(=O)Nc3ccc(C)c(Nc4nccc(-c5cccnc5)n4)c3)cc2)CC1 ; *N(C)c1nccc(N2CC[C@H](NC(=O)Nc3ccc(CN4CCOCC4)c(C(F)(F)F)c3)C2)n1] 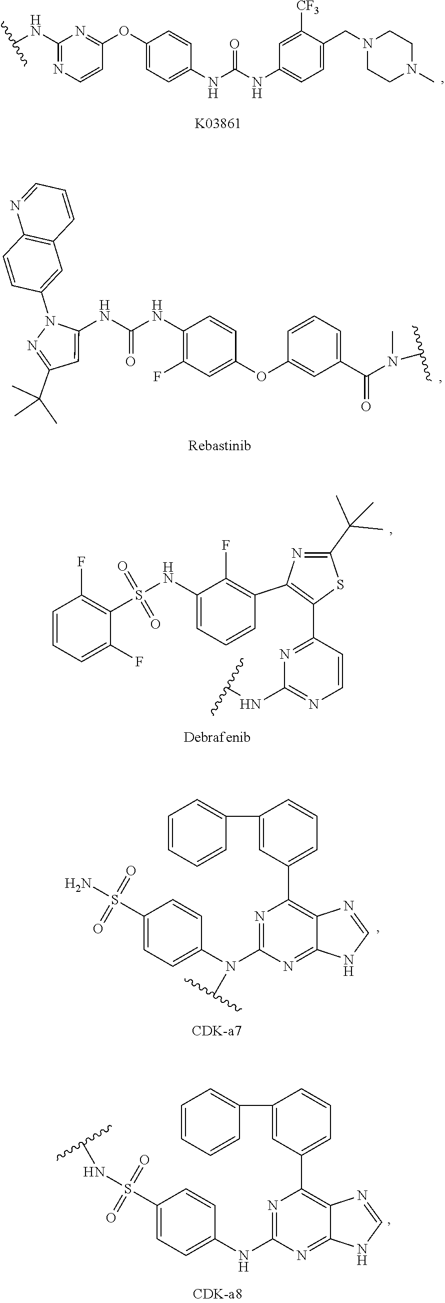 [SMILES: *Nc1nccc(Oc2ccc(NC(=O)Nc3ccc(CN4CCN(C)CC4)c(C(F)(F)F)c3)cc2)n1 ; *N(C)C(=O)c1cccc(Oc2ccc(NC(=O)Nc3cc(C(C)(C)C)nn3-c3ccc4ncccc4c3)c(F)c2)c1 ; *Nc1nccc(-c2sc(C(C)(C)C)nc2-c2cccc(NS(=O)(=O)c3c(F)cccc3F)c2F)n1 ; *N(c1ccc(S(N)(=O)=O)cc1)c1nc(-c2cccc(-c3ccccc3)c2)c2nc[nH]c2n1 ; *NS(=O)(=O)c1ccc(Nc2nc(-c3cccc(-c4ccccc4)c3)c3nc[nH]c3n2)cc1] 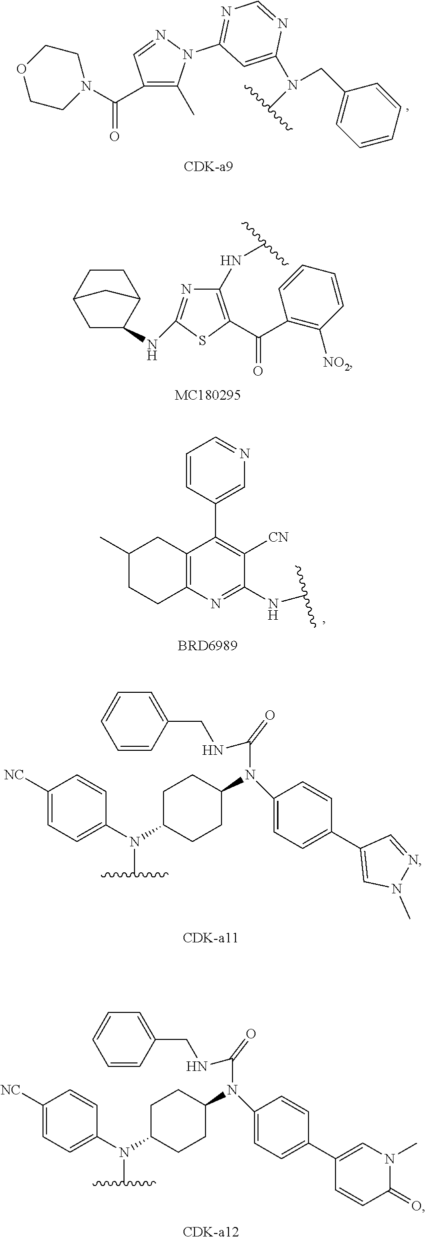 [SMILES: *N(Cc1ccccc1)c1cc(-n2ncc(C(=O)N3CCOCC3)c2C)ncn1 ; *Nc1nc(N[C@H]2CC3CCC2C3)sc1C(=O)c1ccccc1[N+](=O)[O-] ; *Nc1nc2c(c(-c3cccnc3)c1C#N)CC(C)CC2 ; *N(c1ccc(C#N)cc1)[C@H]1CC[C@H](N(C(=O)NCc2ccccc2)c2ccc(-c3cnn(C)c3)cc2)CC1 ; *N(c1ccc(C#N)cc1)[C@H]1CC[C@H](N(C(=O)NCc2ccccc2)c2ccc(-c3ccc(=O)n(C)c3)cc2)CC1] 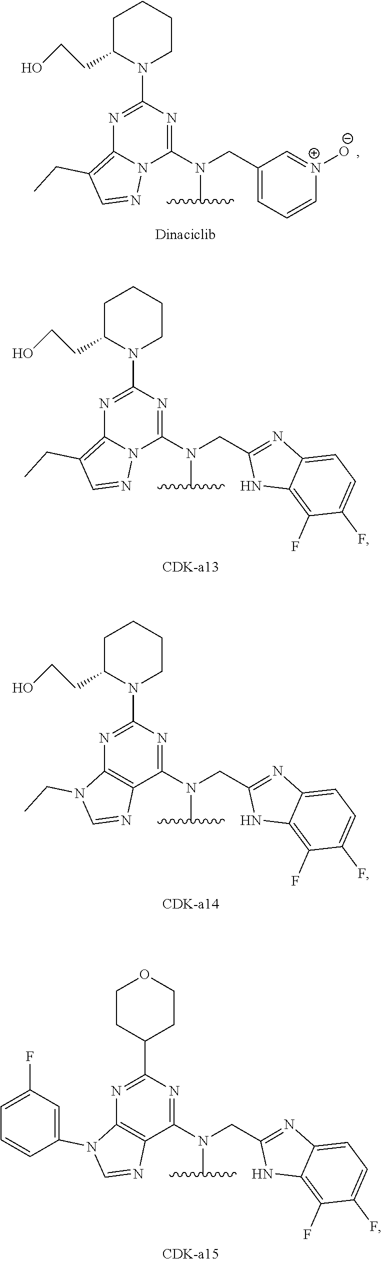 [SMILES: *N(Cc1ccc[n+]([O-])c1)c1nc(N2CCCC[C@H]2CCO)nc2c(CC)cnn12 ; *N(Cc1nc2ccc(F)c(F)c2[nH]1)c1nc(N2CCCC[C@H]2CCO)nc2c(CC)cnn12 ; *N(Cc1nc2ccc(F)c(F)c2[nH]1)c1nc(N2CCCC[C@H]2CCO)nc2c1ncn2CC ; *N(Cc1nc2ccc(F)c(F)c2[nH]1)c1nc(C2CCOCC2)nc2c1ncn2-c1cccc(F)c1] 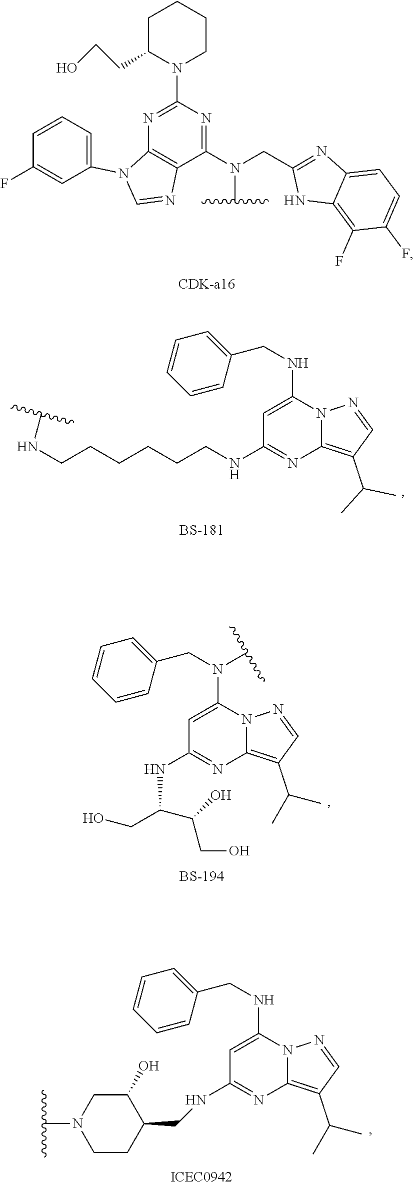 [SMILES: *N(Cc1nc2ccc(F)c(F)c2[nH]1)c1nc(N2CCCC[C@H]2CCO)nc2c1ncn2-c1cccc(F)c1 ; *NCCCCCCNc1cc(NCc2ccccc2)n2ncc(C(C)C)c2n1 ; *N(Cc1ccccc1)c1cc(N[C@@H](CO)[C@H](O)CO)nc2c(C(C)C)cnn12 ; *N1CC[C@H](CNc2cc(NCc3ccccc3)n3ncc(C(C)C)c3n2)[C@@H](O)C1] 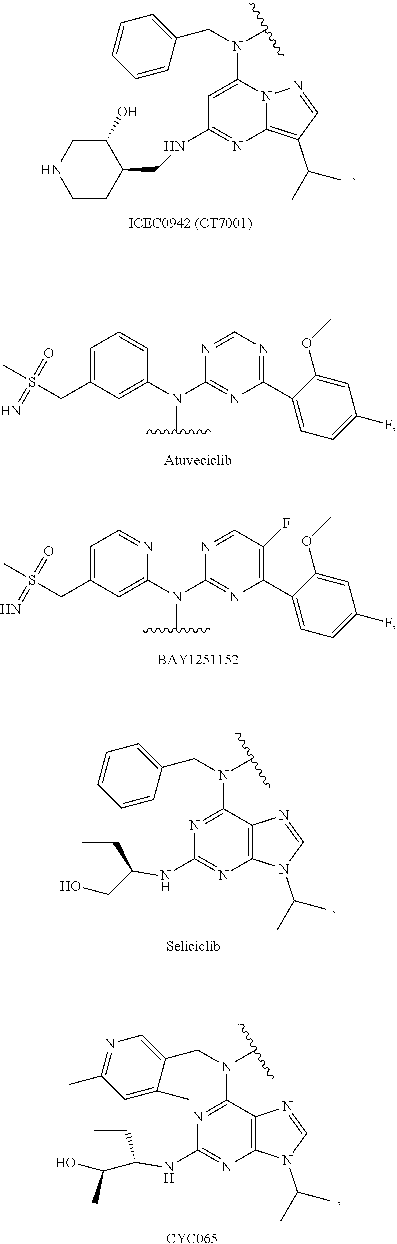 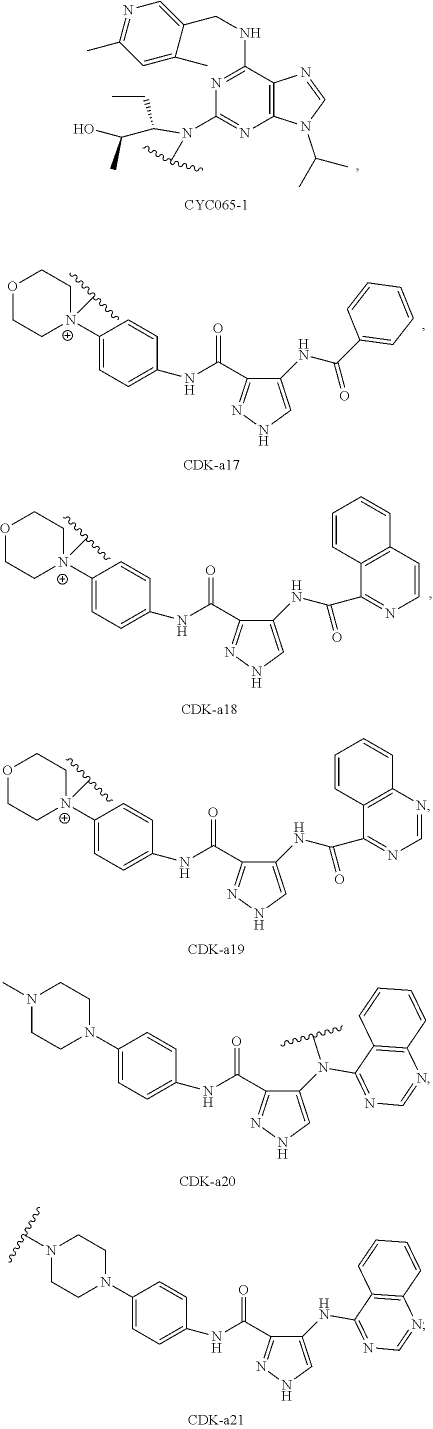
or an isotope of one or more chemical elements, or pharmaceutically acceptable salts, hydrates, or hydrated salts; or the polymorphic crystalline structures of these compounds; or the optical isomers, racemates, diastereomers or enantiomers; [symbol] is a linkage site that links to L₁ and/or L₂ independently.

23. The antibody drug conjugate according to claim 1, wherein the cytotoxic drug, D₁ and D₂, are independently selected from, An EGFR inhibitor, having the following formula: 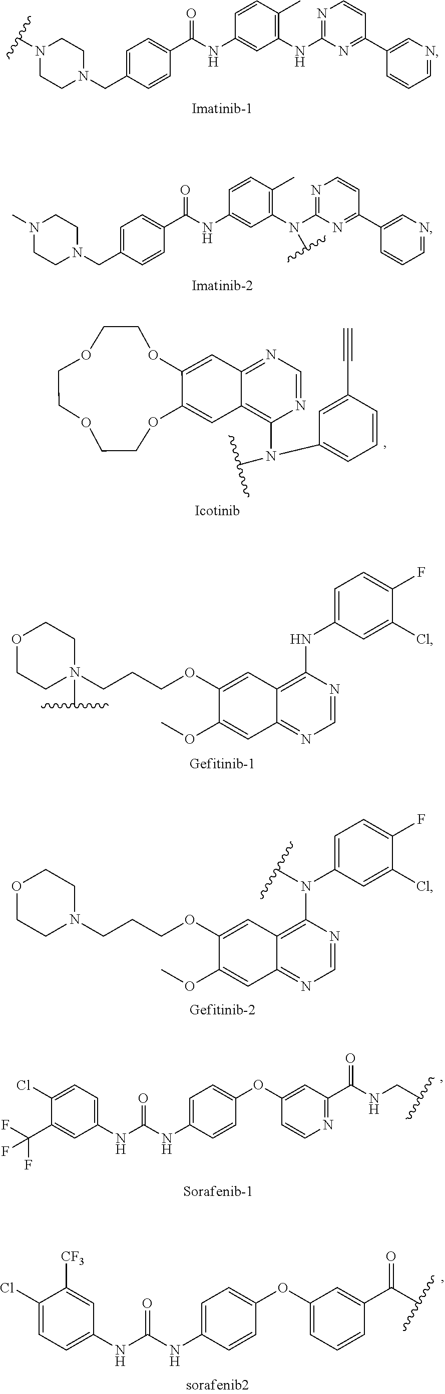 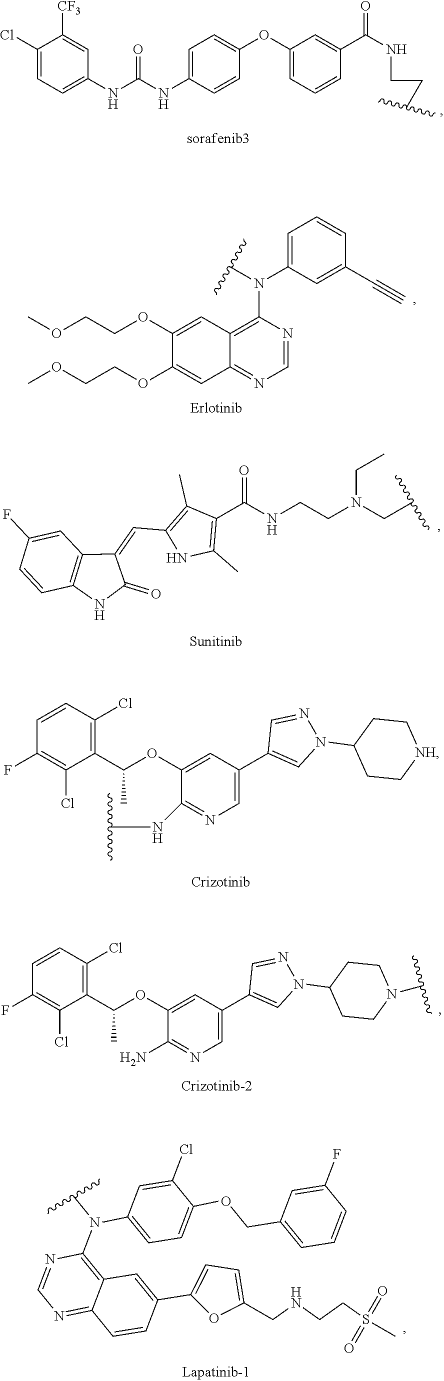 [SMILES: *CCNC(=O)c1cccc(Oc2ccc(NC(=O)Nc3ccc(Cl)c(C(F)(F)F)c3)cc2)c1 ; *N(c1cccc(C#C)c1)c1ncnc2cc(OCCOC)c(OCCOC)cc12 ; *CN(CC)CCNC(=O)c1c(C)[nH]c(/C=C2\C(=O)Nc3ccc(F)cc32)c1C ; *Nc1ncc(-c2cnn(C3CCNCC3)c2)cc1O[C@H](C)c1c(Cl)ccc(F)c1Cl ; *N1CCC(n2cc(-c3cnc(N)c(O[C@H](C)c4c(Cl)ccc(F)c4Cl)c3)cn2)CC1 ; *N(c1ccc(OCc2cccc(F)c2)c(Cl)c1)c1ncnc2ccc(-c3ccc(CNCCS(C)(=O)=O)o3)cc12] 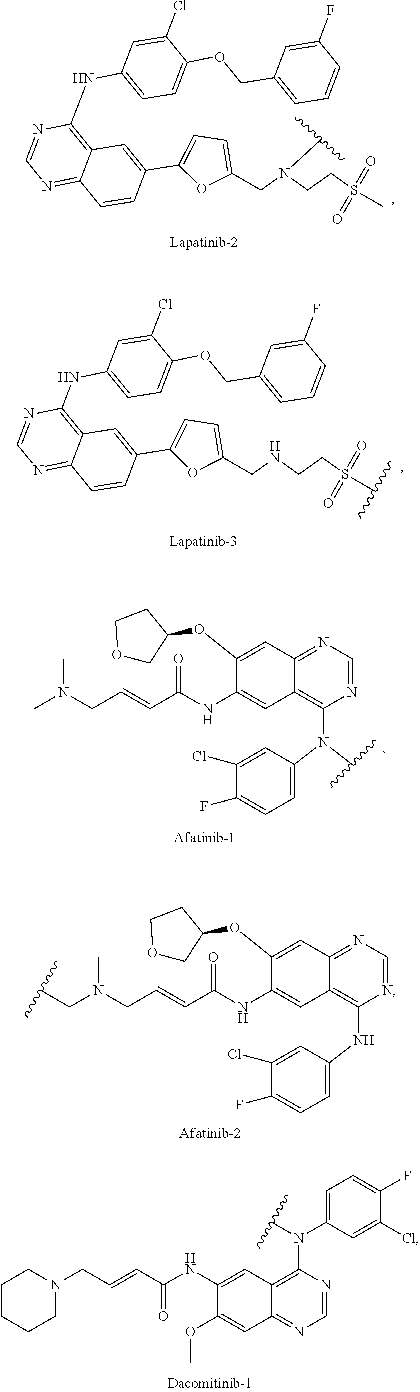 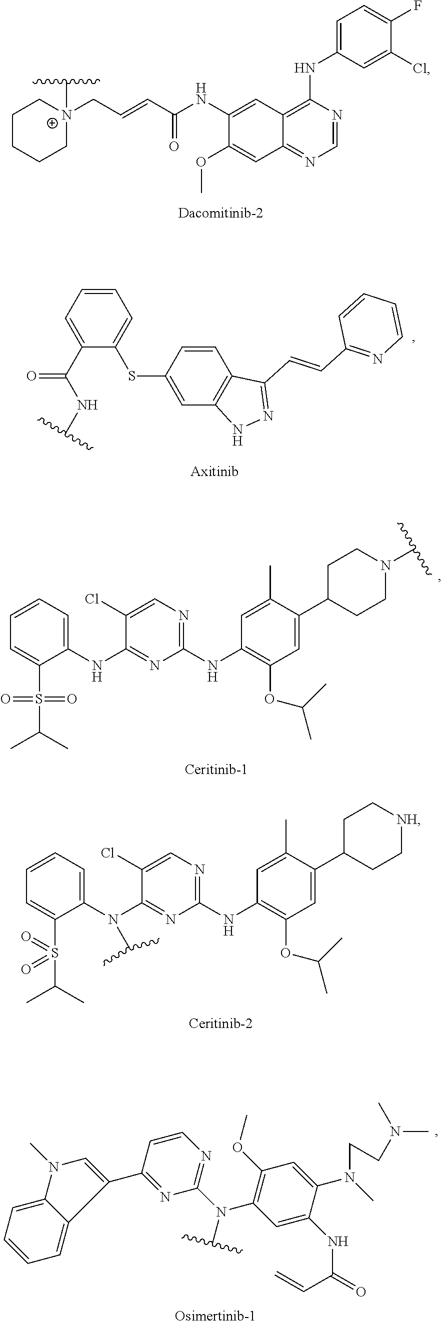 [SMILES: *[N+]1(C/C=C/C(=O)Nc2cc3c(Nc4ccc(F)c(Cl)c4)ncnc3cc2OC)CCCCC1 ; *NC(=O)c1ccccc1Sc1ccc2c(/C=C/c3ccccn3)n[nH]c2c1 ; *N1CCC(c2cc(OC(C)C)c(Nc3ncc(Cl)c(Nc4ccccc4S(=O)(=O)C(C)C)n3)cc2C)CC1 ; *N(c1ccccc1S(=O)(=O)C(C)C)c1nc(Nc2cc(C)c(C3CCNCC3)cc2OC(C)C)ncc1Cl ; *N(c1nccc(-c2cn(C)c3ccccc23)n1)c1cc(NC(=O)C=C)c(N(C)CCN(C)C)cc1OC] 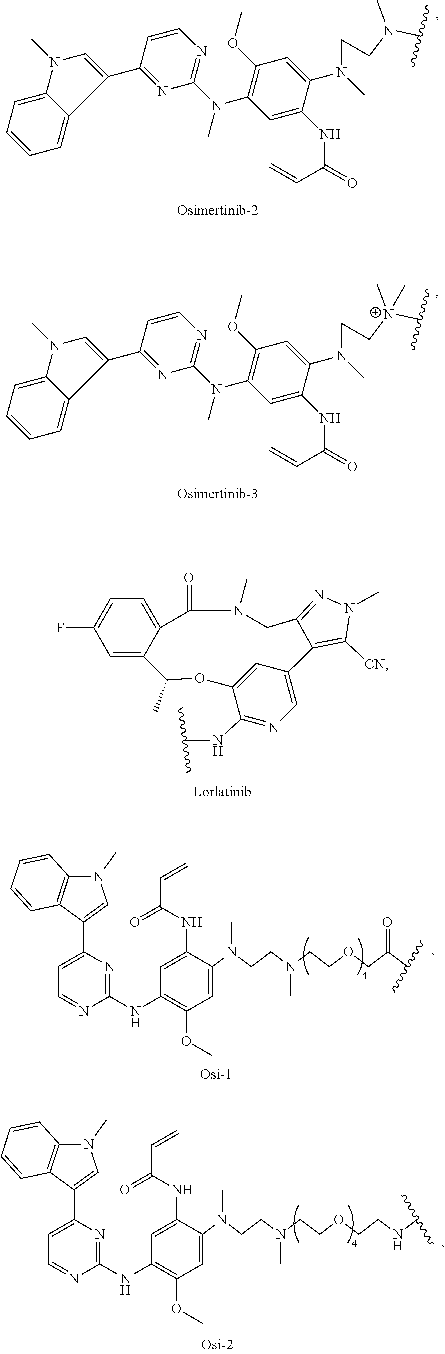 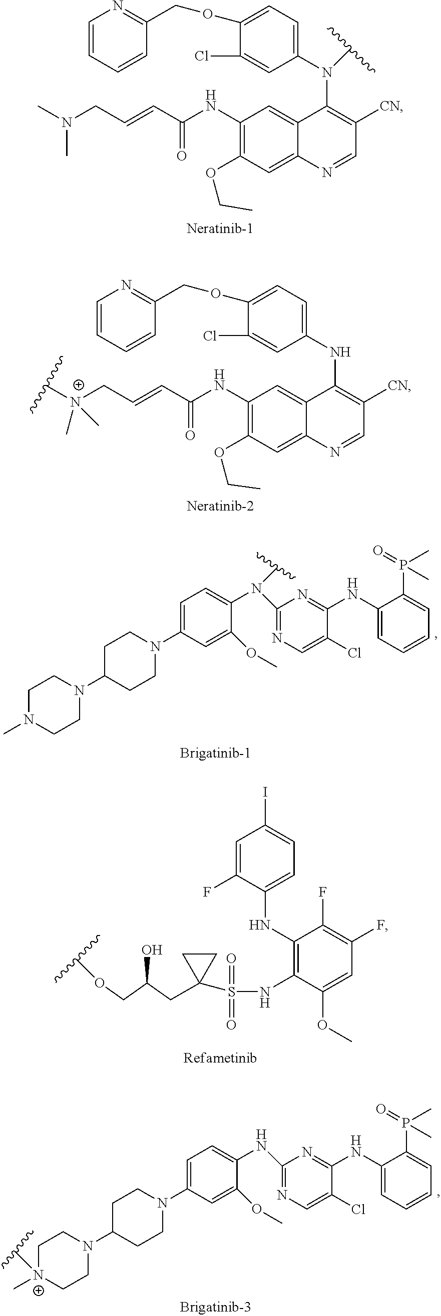 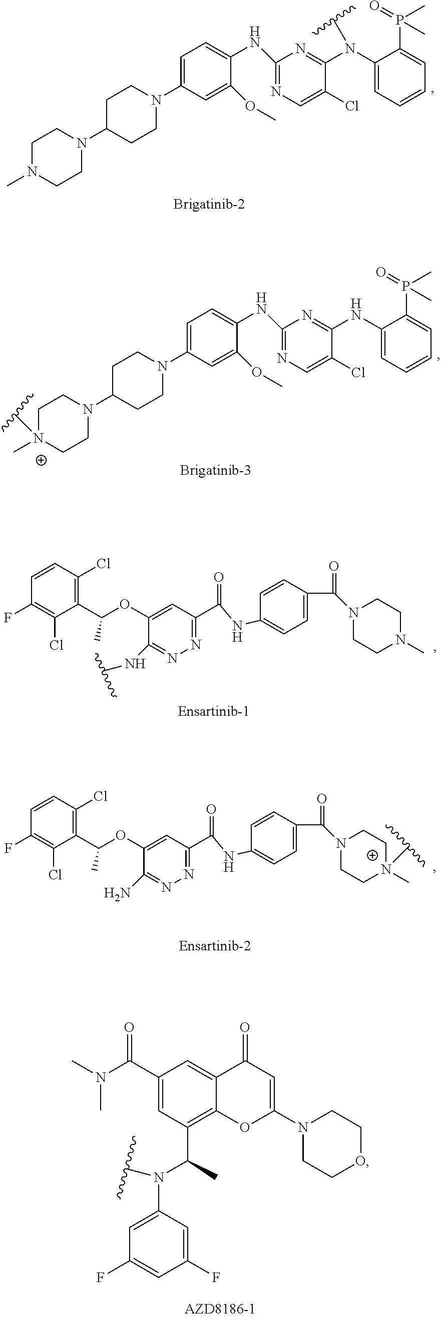 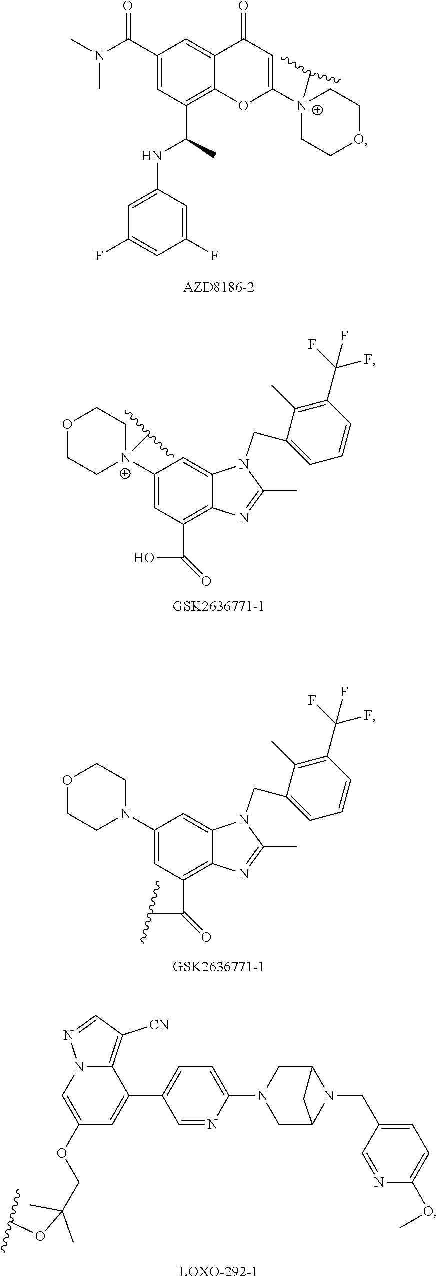 [SMILES: *[N+]1(c2cc(=O)c3cc(C(=O)N(C)C)cc([C@@H](C)Nc4cc(F)cc(F)c4)c3o2)CCOCC1 ; *[N+]1(c2cc(C(=O)O)c3nc(C)n(Cc4cccc(C(F)(F)F)c4C)c3c2)CCOCC1 ; *C(=O)c1cc(N2CCOCC2)cc2c1nc(C)n2Cc1cccc(C(F)(F)F)c1C ; *OC(C)(C)COc1cc(-c2ccc(N3CC4CC(C3)N4Cc3ccc(OC)nc3)nc2)c2c(C#N)cnn2c1] 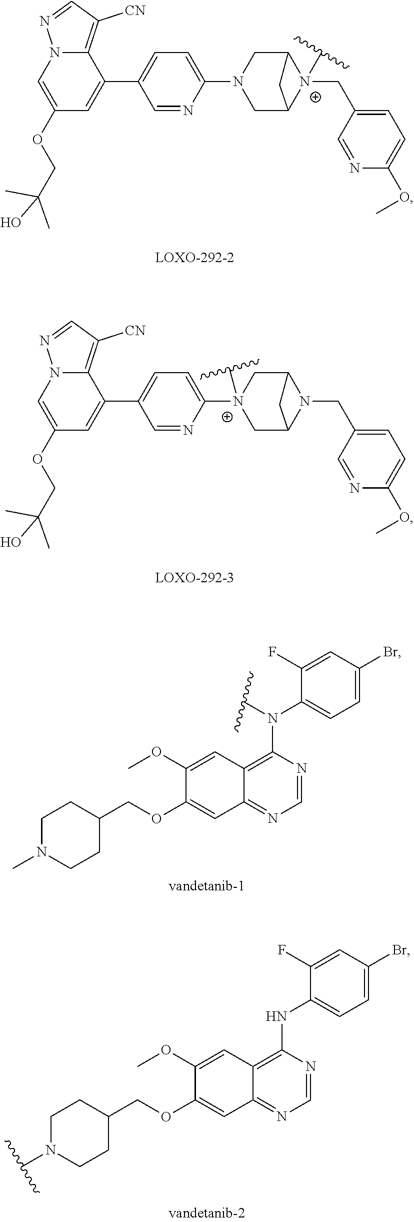 [SMILES: *[N+]1(Cc2ccc(OC)nc2)C2CC1CN(c1ccc(-c3cc(OCC(C)(C)O)cn4ncc(C#N)c34)cn1)C2 ; *[N+]1(c2ccc(-c3cc(OCC(C)(C)O)cn4ncc(C#N)c34)cn2)CC2CC(C1)N2Cc1ccc(OC)nc1 ; *N(c1ccc(Br)cc1F)c1ncnc2cc(OCC3CCN(C)CC3)c(OC)cc12 ; *N1CCC(COc2cc3ncnc(Nc4ccc(Br)cc4F)c3cc2OC)CC1] 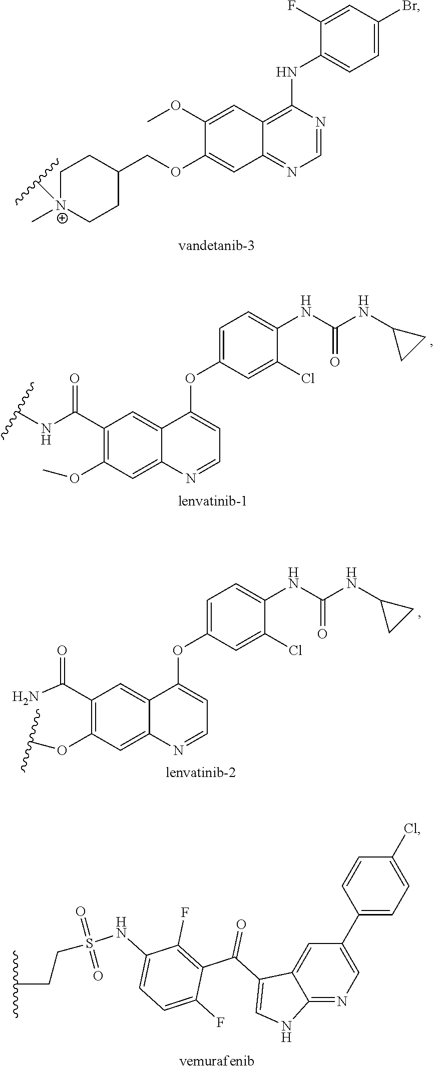 [SMILES: *[N+]1(C)CCC(COc2cc3ncnc(Nc4ccc(Br)cc4F)c3cc2OC)CC1 ; *NC(=O)c1cc2c(Oc3ccc(NC(=O)NC4CC4)c(Cl)c3)ccnc2cc1OC ; *Oc1cc2nccc(Oc3ccc(NC(=O)NC4CC4)c(Cl)c3)c2cc1C(N)=O ; *CCS(=O)(=O)Nc1ccc(F)c(C(=O)c2c[nH]c3ncc(-c4ccc(Cl)cc4)cc23)c1F] 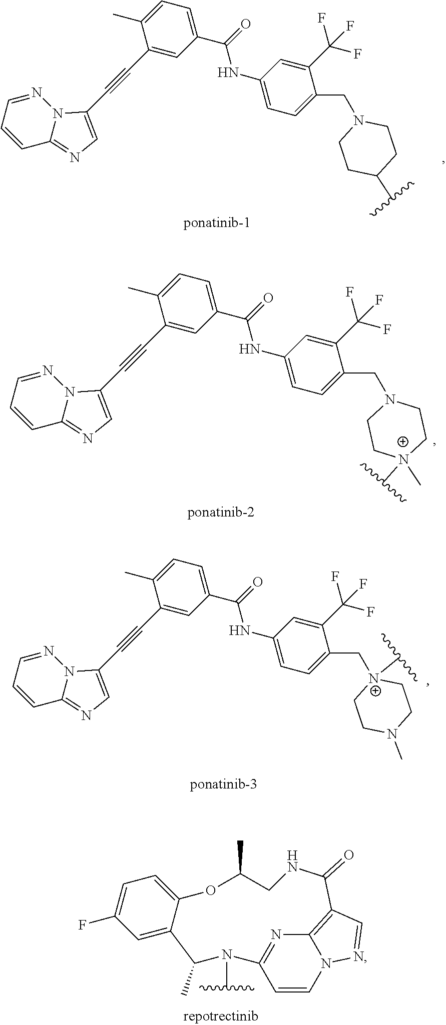 [SMILES: *C1CCN(Cc2ccc(NC(=O)c3ccc(C)c(C#Cc4cnc5cccnn45)c3)cc2C(F)(F)F)CC1 ; *[N+]1(C)CCN(Cc2ccc(NC(=O)c3ccc(C)c(C#Cc4cnc5cccnn45)c3)cc2C(F)(F)F)CC1 ; *[N+]1(Cc2ccc(NC(=O)c3ccc(C)c(C#Cc4cnc5cccnn45)c3)cc2C(F)(F)F)CCN(C)CC1 ; *N1c2ccn3ncc(c3n2)C(=O)NC[C@H](C)Oc2ccc(F)cc2[C@H]1C] 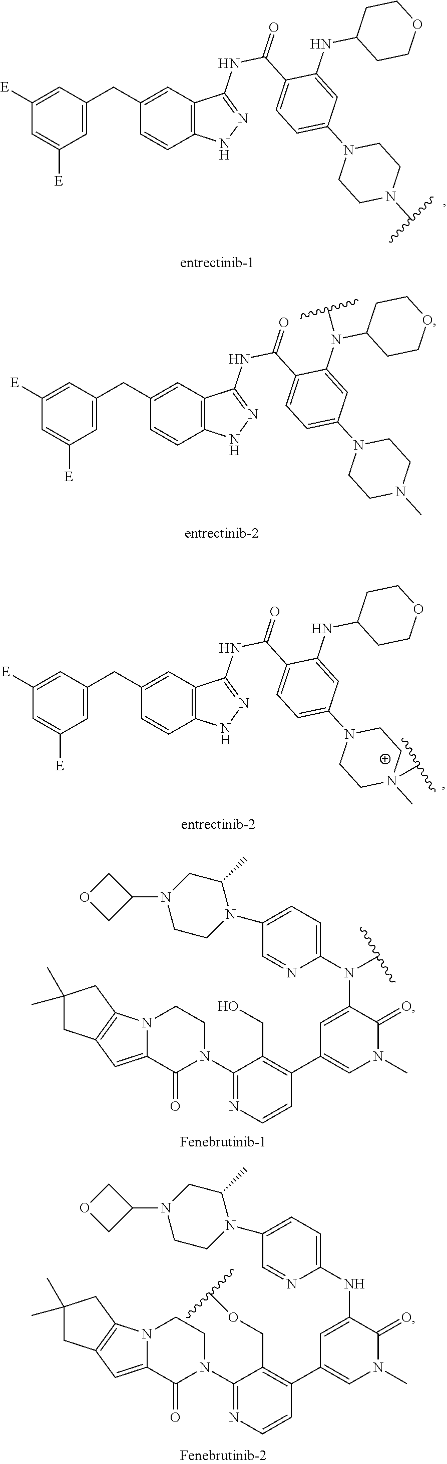 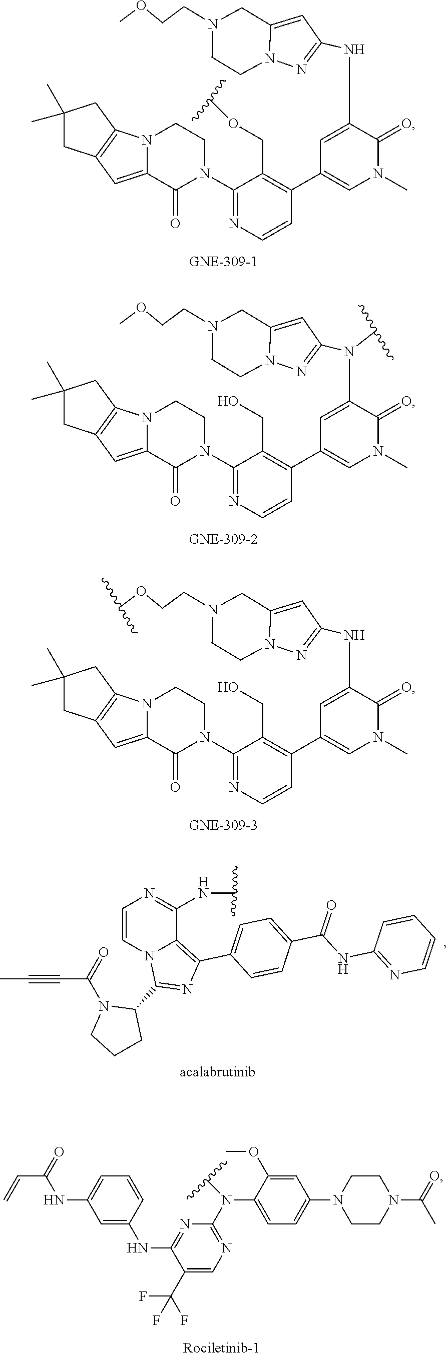 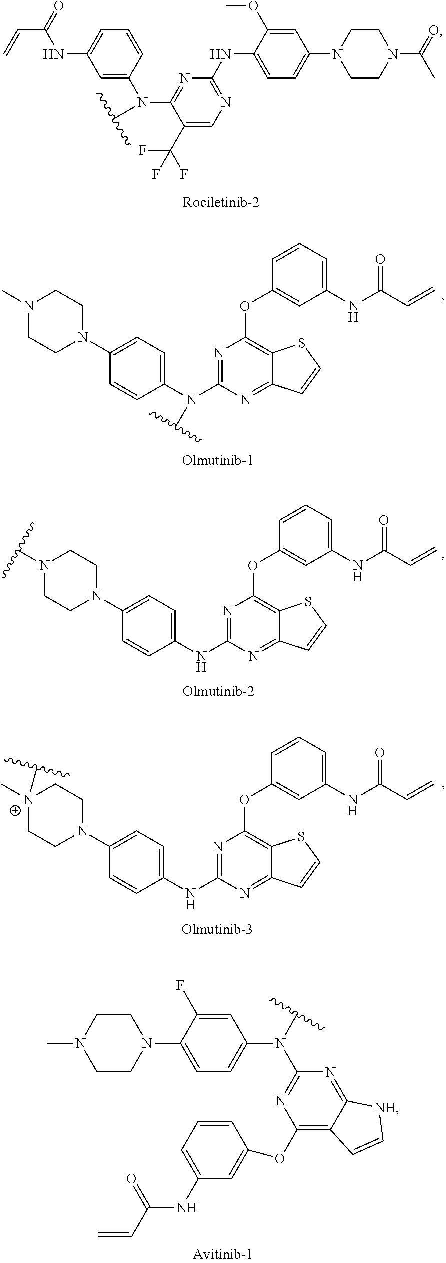 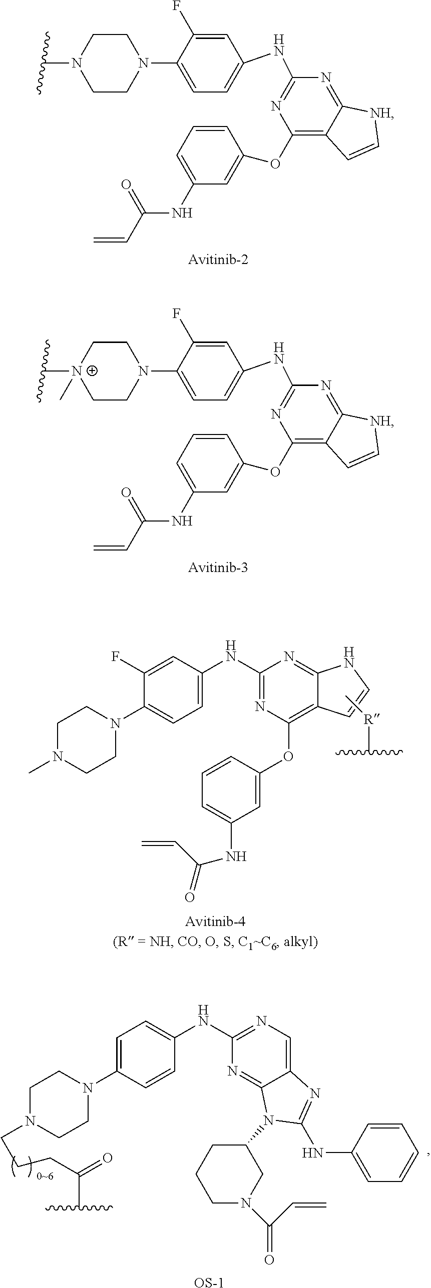 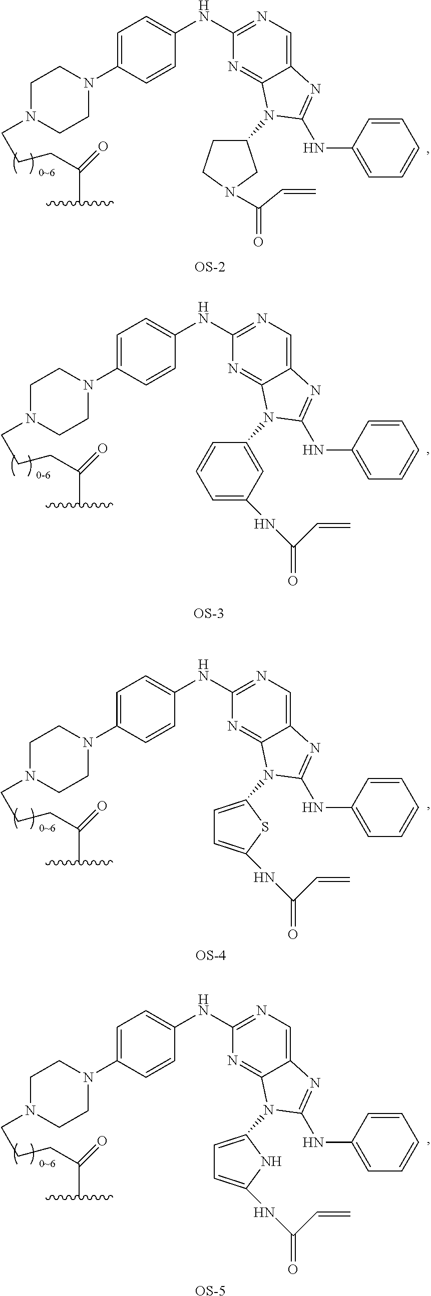 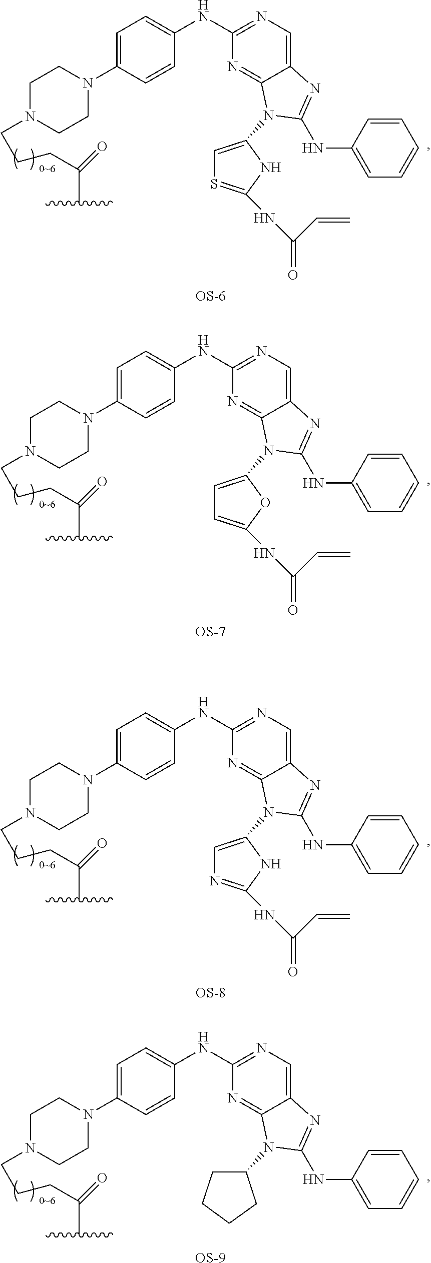 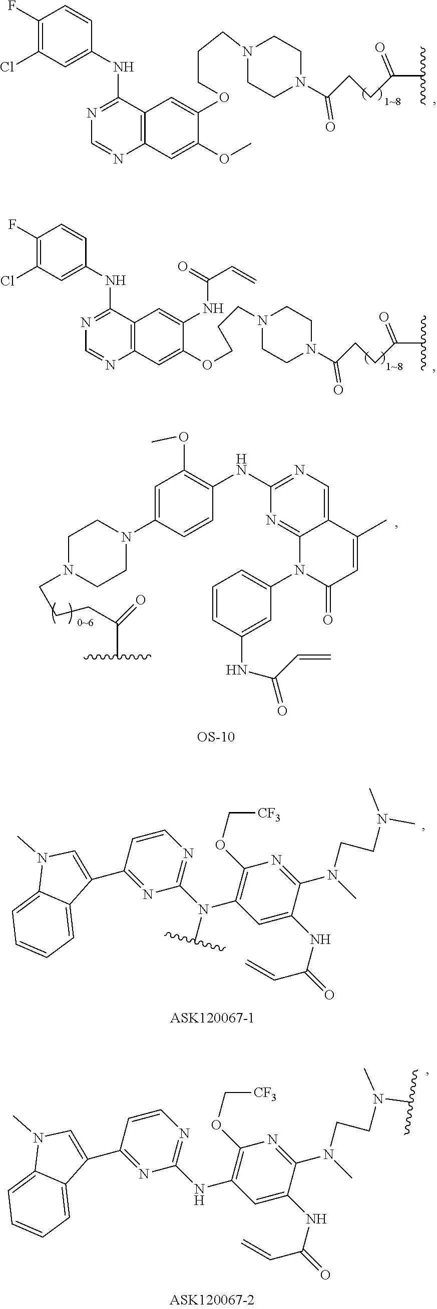 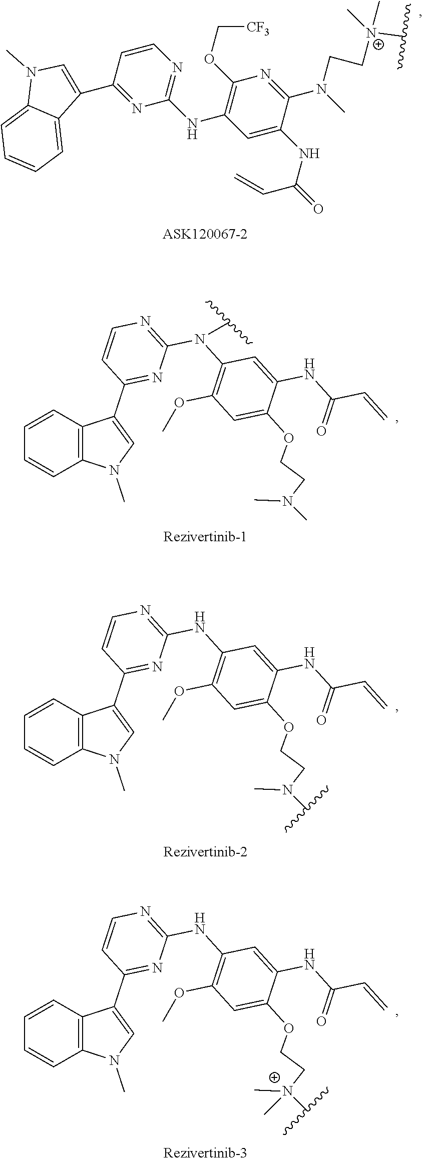 [SMILES: *[N+](C)(C)CCN(C)c1nc(OCC(F)(F)F)c(Nc2nccc(-c3cn(C)c4ccccc34)n2)cc1NC(=O)C=C ; *N(c1nccc(-c2cn(C)c3ccccc23)n1)c1cc(NC(=O)C=C)c(OCCN(C)C)cc1OC ; *N(C)CCOc1cc(OC)c(Nc2nccc(-c3cn(C)c4ccccc34)n2)cc1NC(=O)C=C ; *[N+](C)(C)CCOc1cc(OC)c(Nc2nccc(-c3cn(C)c4ccccc34)n2)cc1NC(=O)C=C] 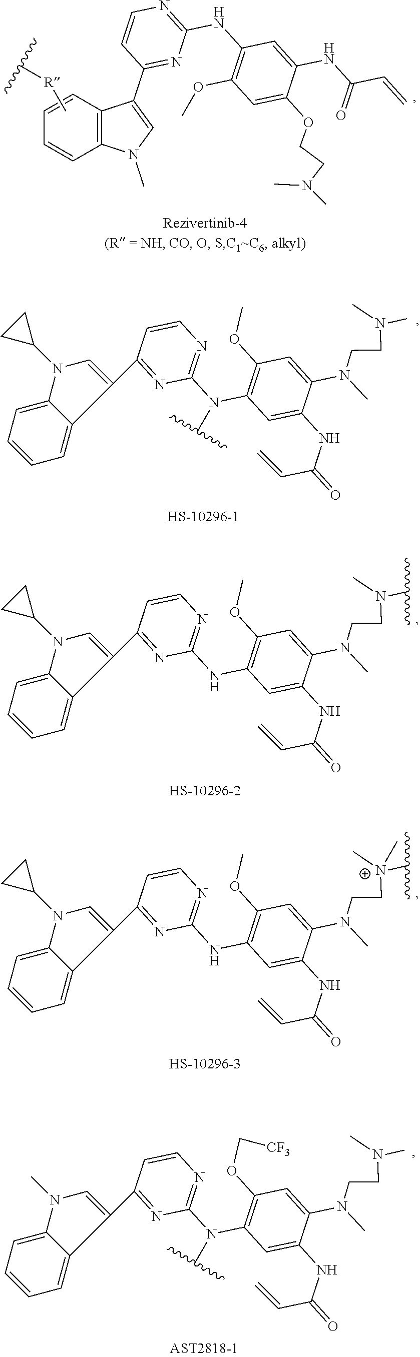 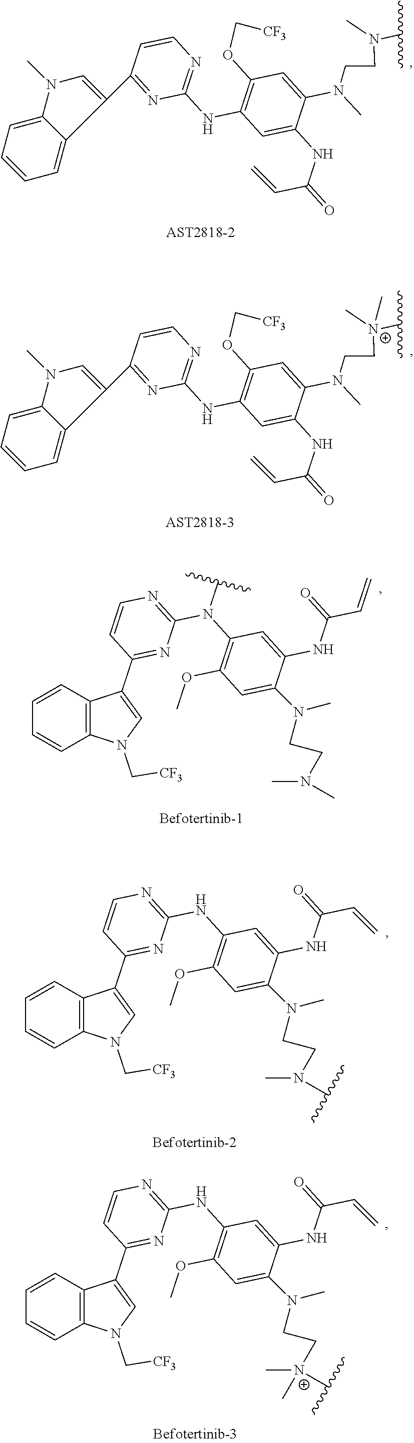 [SMILES: *N(C)CCN(C)c1cc(OCC(F)(F)F)c(Nc2nccc(-c3cn(C)c4ccccc34)n2)cc1NC(=O)C=C ; *[N+](C)(C)CCN(C)c1cc(OCC(F)(F)F)c(Nc2nccc(-c3cn(C)c4ccccc34)n2)cc1NC(=O)C=C ; *N(c1nccc(-c2cn(CC(F)(F)F)c3ccccc23)n1)c1cc(NC(=O)C=C)c(N(C)CCN(C)C)cc1OC ; *N(C)CCN(C)c1cc(OC)c(Nc2nccc(-c3cn(CC(F)(F)F)c4ccccc34)n2)cc1NC(=O)C=C ; *[N+](C)(C)CCN(C)c1cc(OC)c(Nc2nccc(-c3cn(CC(F)(F)F)c4ccccc34)n2)cc1NC(=O)C=C] 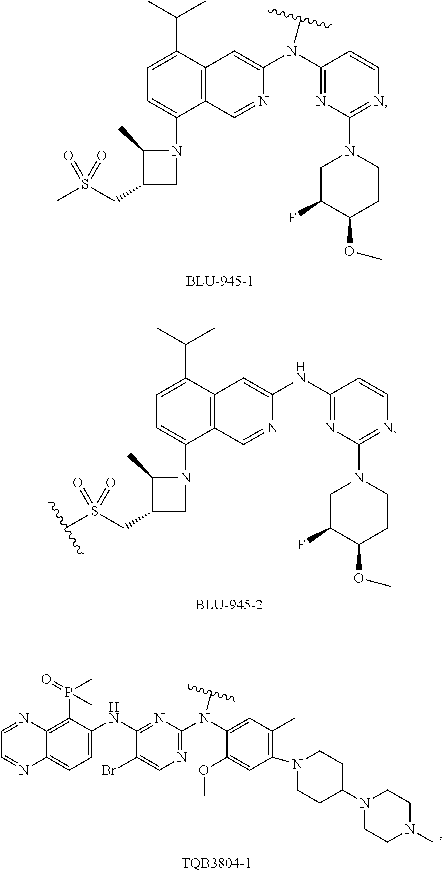 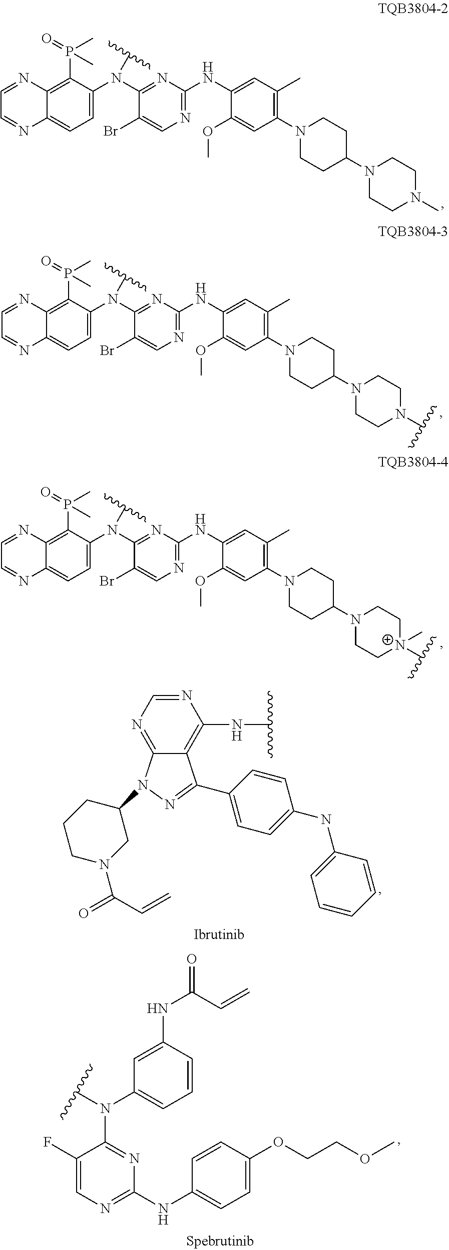 [SMILES: *N(c1ccc2nccnc2c1P(C)(C)=O)c1nc(Nc2cc(C)c(N3CCC(N4CCN(C)CC4)CC3)cc2OC)ncc1Br ; *N1CCN(C2CCN(c3cc(OC)c(Nc4ncc(Br)c(N(*)c5ccc6nccnc6c5P(C)(C)=O)n4)cc3C)CC2)CC1 ; *N(c1ccc2nccnc2c1P(C)(C)=O)c1nc(Nc2cc(C)c(N3CCC(N4CC[N+](*)(C)CC4)CC3)cc2OC)ncc1Br ; *Nc1ncnc2c1c(-c1ccc(Nc3ccccc3)cc1)nn2[C@@H]1CCCN(C(=O)C=C)C1 ; *N(c1cccc(NC(=O)C=C)c1)c1nc(Nc2ccc(OCCOC)cc2)ncc1F] 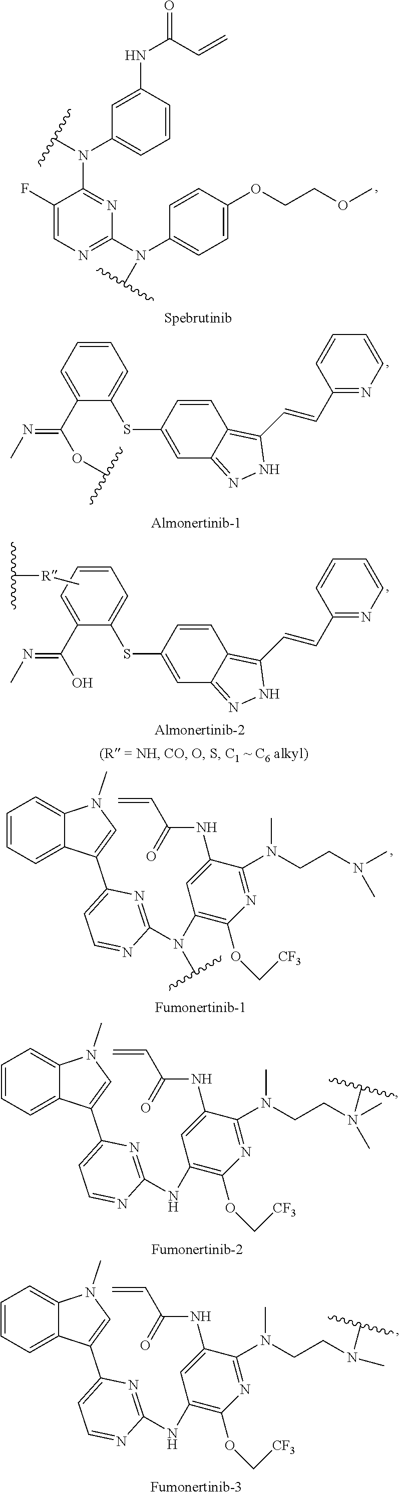 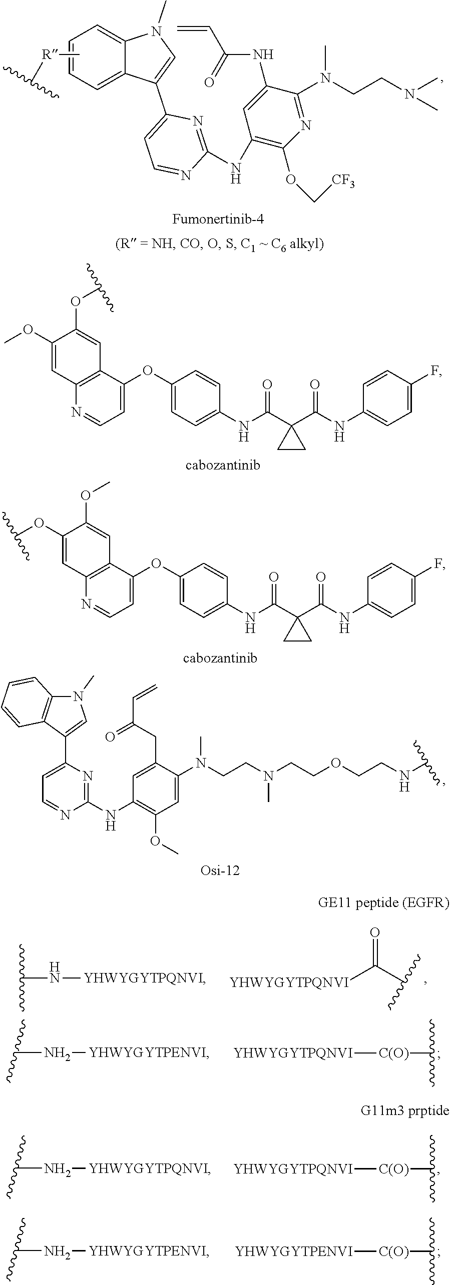
or an isotope of one or more chemical elements, or pharmaceutically acceptable salts, hydrates, or hydrated salts; or the polymorphic crystalline structures of these compounds; or the optical isomers, racemates, diastereomers or enantiomers; [symbol] is a linkage site that links to L₁ and/or L₂ independently.

24. The antibody drug conjugate according to claim 1, wherein the cytotoxic drug, D₁ and D₂, are independently selected from, A PLK1 inhibitor, having the following formula: 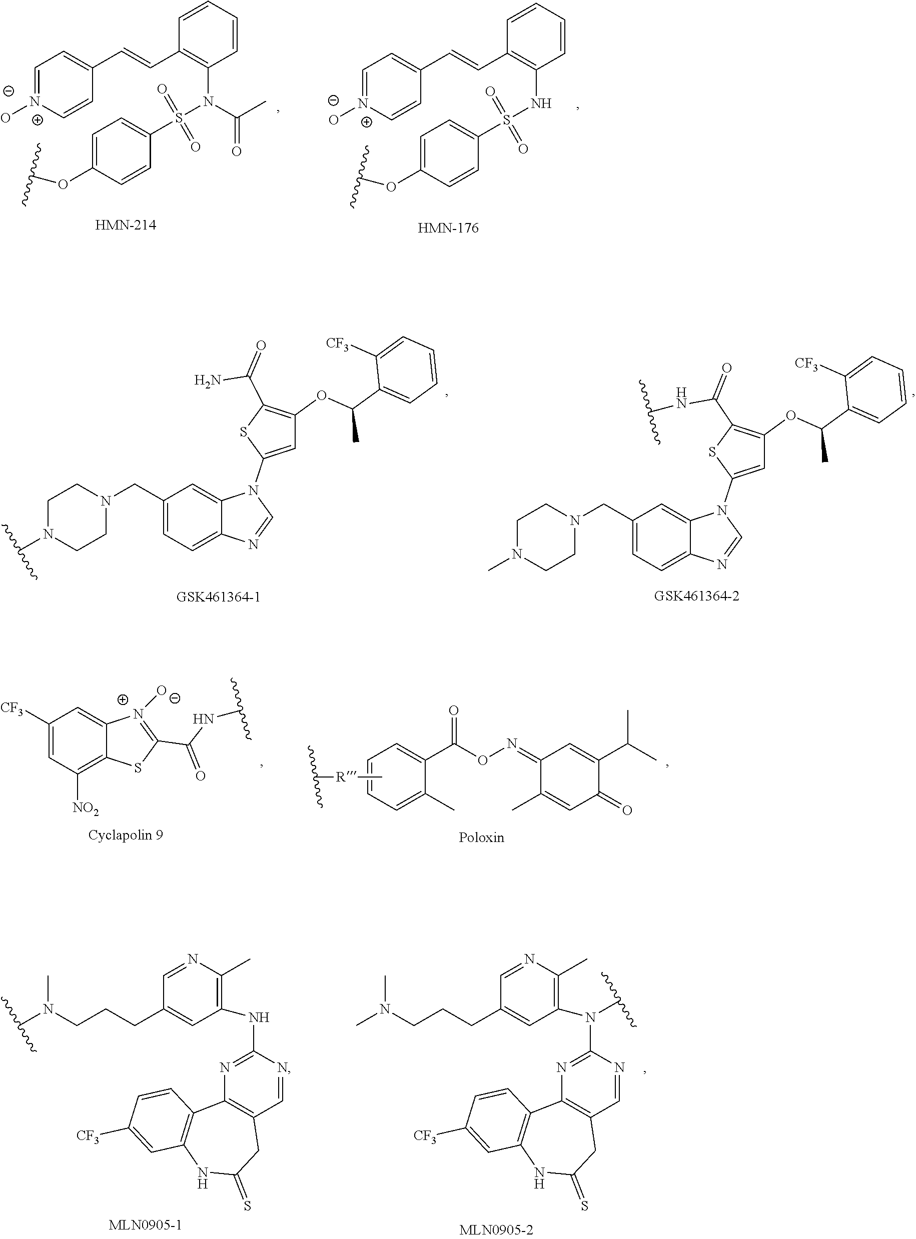 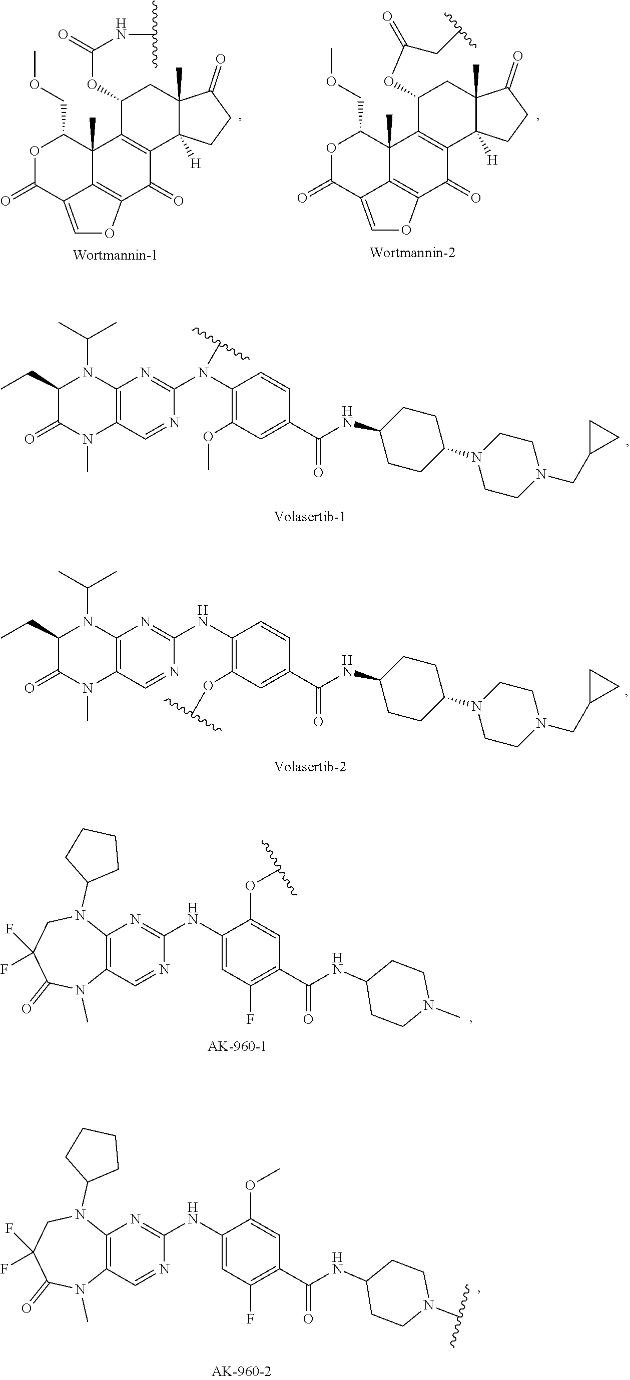 [SMILES: *NC(=O)O[C@@H]1C[C@]2(C)C(=O)CC[C@H]2C2=C1[C@]1(C)c3c(coc3C2=O)C(=O)O[C@@H]1COC ; *CC(=O)O[C@@H]1C[C@]2(C)C(=O)CC[C@H]2C2=C1[C@]1(C)c3c(coc3C2=O)C(=O)O[C@@H]1COC ; *N(c1ncc2c(n1)N(C(C)C)[C@H](CC)C(=O)N2C)c1ccc(C(=O)N[C@H]2CC[C@H](N3CCN(CC4CC4)CC3)CC2)cc1OC ; *Oc1cc(C(=O)N[C@H]2CC[C@H](N3CCN(CC4CC4)CC3)CC2)ccc1Nc1ncc2c(n1)N(C(C)C)[C@H](CC)C(=O)N2C ; *Oc1cc(C(=O)NC2CCN(C)CC2)c(F)cc1Nc1ncc2c(n1)N(C1CCCC1)CC(F)(F)C(=O)N2C ; *N1CCC(NC(=O)c2cc(OC)c(Nc3ncc4c(n3)N(C3CCCC3)CC(F)(F)C(=O)N4C)cc2F)CC1] 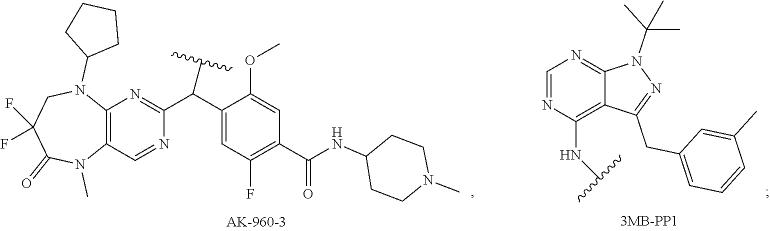 [SMILES: *C(c1ncc2c(n1)N(C1CCCC1)CC(F)(F)C(=O)N2C)c1cc(F)c(C(=O)NC2CCN(C)CC2)cc1OC ; *Nc1ncnc2c1c(Cc1cccc(C)c1)nn2C(C)(C)C]
or hydrated salts; or the polymorphic crystalline structures of these compounds; or the optical isomers, racemates, diastereomers or enantiomers; [symbol] is a linkage site that links to L₁ and/or L₂ independently.

25. The antibody drug conjugate according to claim 1, wherein the cytotoxic drug, D₁ and D₂, are independently selected from, a PI3K/AKT/mTOR inhibitor, having the following formula: 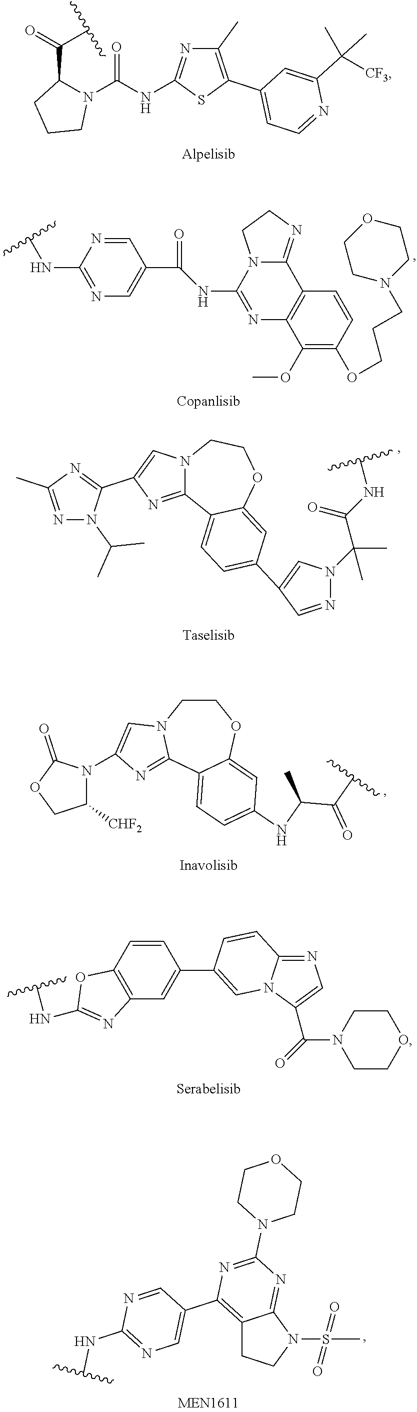 [SMILES: *C(=O)[C@@H]1CCCN1C(=O)Nc1nc(C)c(-c2ccnc(C(C)(C)C(F)(F)F)c2)s1 ; *Nc1ncc(C(=O)NC2=Nc3c(ccc(OCCCN4CCOCC4)c3OC)C3=NCCN23)cn1 ; *NC(=O)C(C)(C)n1cc(-c2ccc3c(c2)OCCn2cc(-c4nc(C)nn4C(C)C)nc2-3)cn1 ; *C(=O)[C@H](C)Nc1ccc2c(c1)OCCn1cc(N3C(=O)OC[C@H]3C(F)F)nc1-2 ; *Nc1nc2cc(-c3ccc4ncc(C(=O)N5CCOCC5)n4c3)ccc2o1 ; *Nc1ncc(-c2nc(N3CCOCC3)nc3c2CCN3S(C)(=O)=O)cn1] 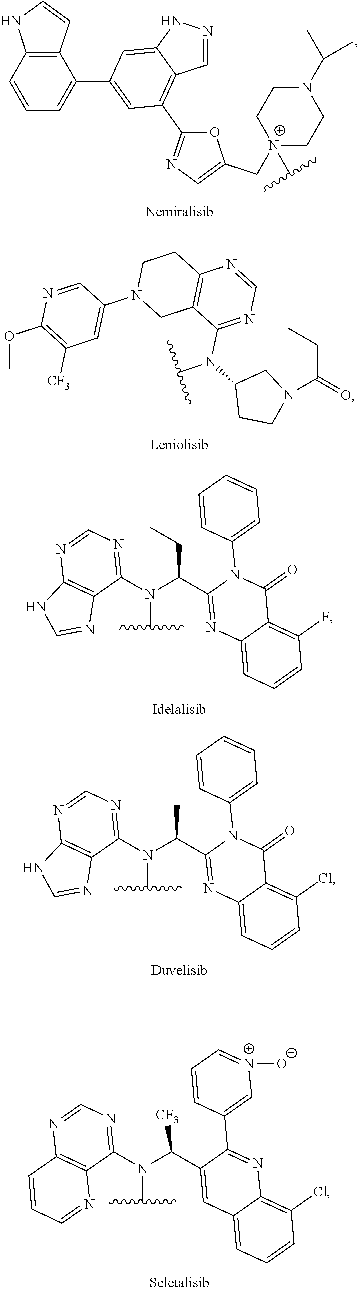 [SMILES: *[N+]1(Cc2cnc(-c3cc(-c4cccc5[nH]ccc45)cc4[nH]ncc34)o2)CCN(C(C)C)CC1 ; *N(c1ncnc2c1CN(c1cnc(OC)c(C(F)(F)F)c1)CC2)[C@H]1CCN(C(=O)CC)C1 ; *N(c1ncnc2[nH]cnc12)[C@@H](CC)c1nc2cccc(F)c2c(=O)n1-c1ccccc1 ; *N(c1ncnc2[nH]cnc12)[C@@H](C)c1nc2cccc(Cl)c2c(=O)n1-c1ccccc1 ; *N(c1ncnc2cccnc12)[C@H](c1cc2cccc(Cl)c2nc1-c1ccc[n+]([O-])c1)C(F)(F)F] 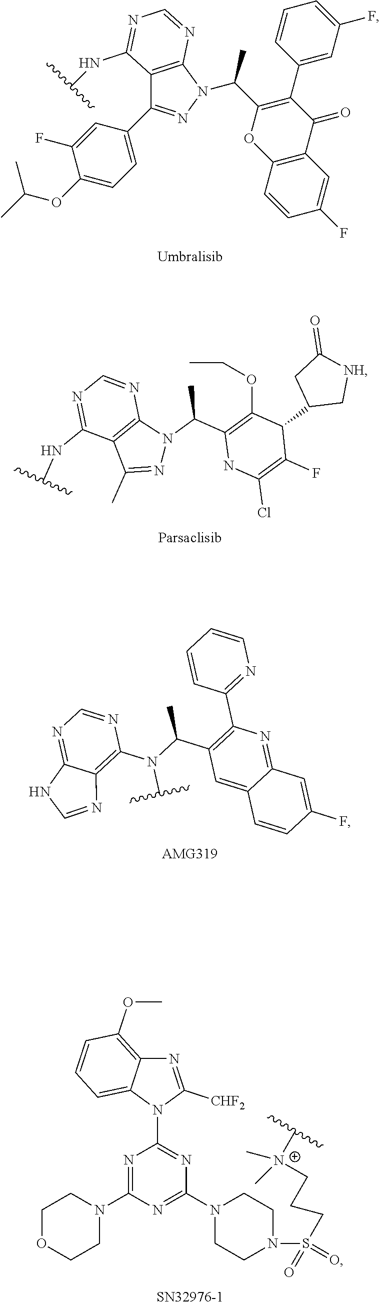 [SMILES: *Nc1ncnc2c1c(-c1ccc(OC(C)C)c(F)c1)nn2[C@@H](C)c1oc2ccc(F)cc2c(=O)c1-c1cccc(F)c1 ; *Nc1ncnc2c1c(C)nn2[C@@H](C)C1=C(OCC)[C@H](C2CNC(=O)C2)C(F)=C(Cl)N1 ; *N(c1ncnc2[nH]cnc12)[C@@H](C)c1cc2ccc(F)cc2nc1-c1ccccn1 ; *[N+](C)(C)CCCS(=O)(=O)N1CCN(c2nc(N3CCOCC3)nc(-n3c(C(F)F)nc4c(OC)cccc43)n2)CC1] 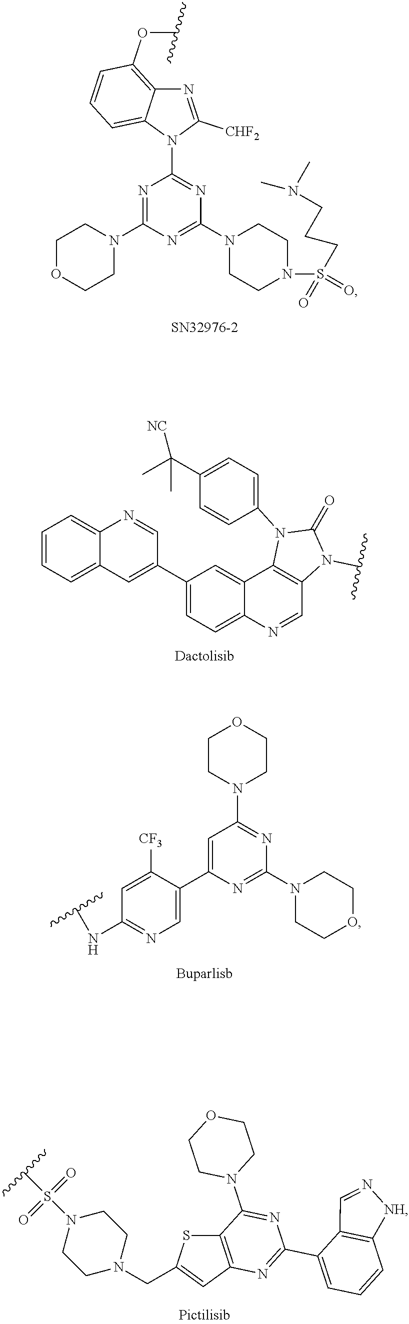 [SMILES: *Oc1cccc2c1nc(C(F)F)n2-c1nc(N2CCOCC2)nc(N2CCN(S(=O)(=O)CCCN(C)C)CC2)n1 ; *n1c(=O)n(-c2ccc(C(C)(C)C#N)cc2)c2c3cc(-c4cnc5ccccc5c4)ccc3ncc21 ; *Nc1cc(C(F)(F)F)c(-c2cc(N3CCOCC3)nc(N3CCOCC3)n2)cn1 ; *S(=O)(=O)N1CCN(Cc2cc3nc(-c4cccc5[nH]ncc45)nc(N4CCOCC4)c3s2)CC1] 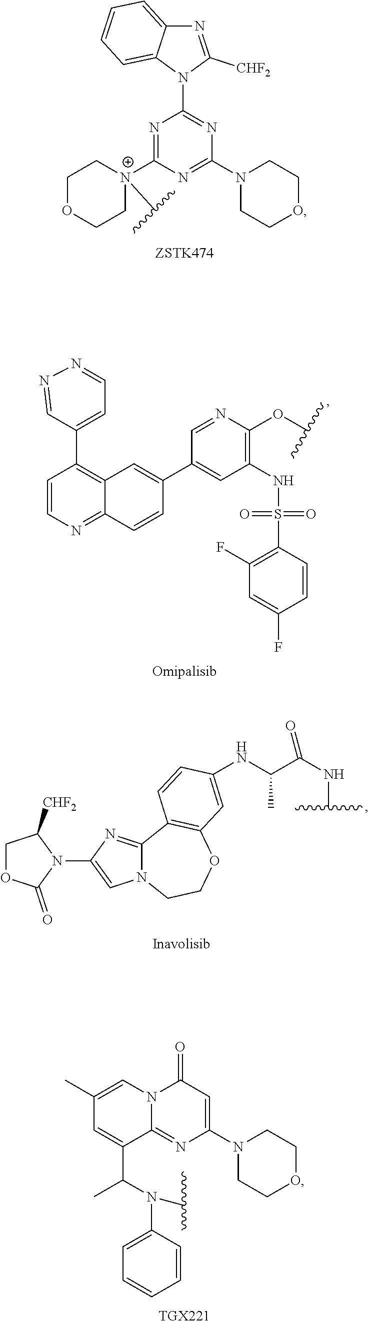 [SMILES: *[N+]1(c2nc(N3CCOCC3)nc(-n3c(C(F)F)nc4ccccc43)n2)CCOCC1 ; *Oc1ncc(-c2ccc3nccc(-c4ccnnc4)c3c2)cc1NS(=O)(=O)c1ccc(F)cc1F ; *NC(=O)[C@H](C)Nc1ccc2c(c1)OCCn1cc(N3C(=O)OC[C@H]3C(F)F)nc1-2 ; *N(c1ccccc1)C(C)c1cc(C)cn2c(=O)cc(N3CCOCC3)nc12] 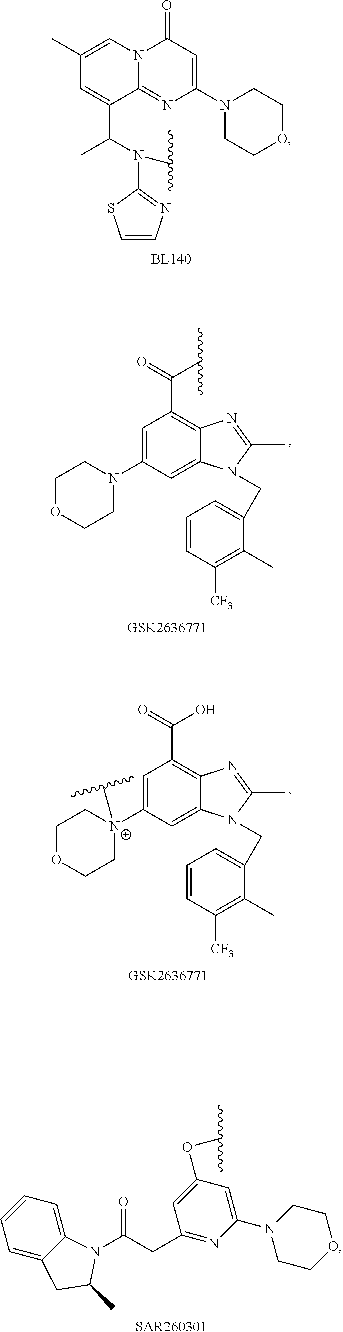 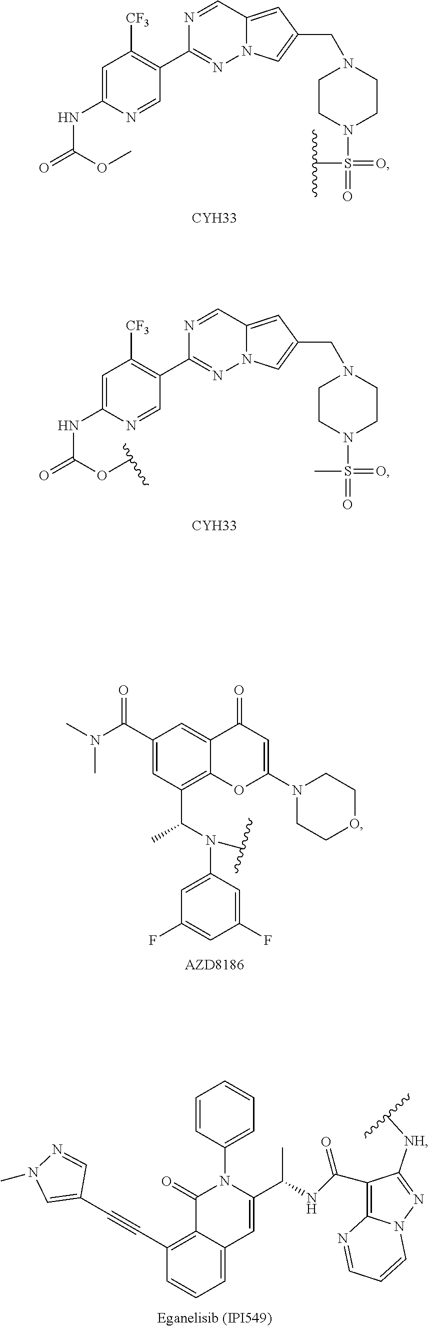 [SMILES: *S(=O)(=O)N1CCN(Cc2cc3cnc(-c4cnc(NC(=O)OC)cc4C(F)(F)F)nn3c2)CC1 ; "CYH33" ; *OC(=O)Nc1cc(C(F)(F)F)c(-c2ncc3cc(CN4CCN(S(C)(=O)=O)CC4)cn3n2)cn1 ; "CYH33" ; *N(c1cc(F)cc(F)c1)[C@H](C)c1cc(C(=O)N(C)C)cc2c(=O)cc(N3CCOCC3)oc12 ; *Nc1nn2cccnc2c1C(=O)N[C@@H](C)c1cc2cccc(C#Cc3cnn(C)c3)c2c(=O)n1-c1ccccc1] 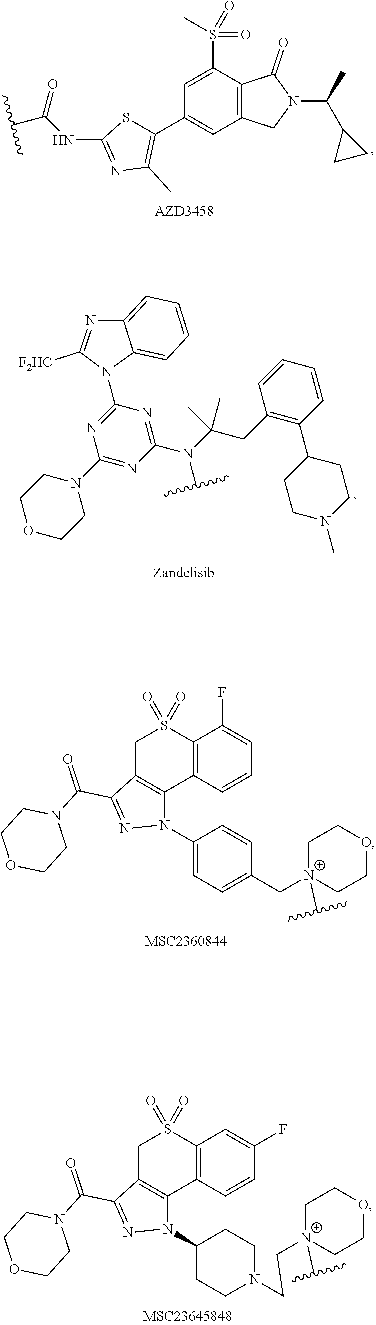 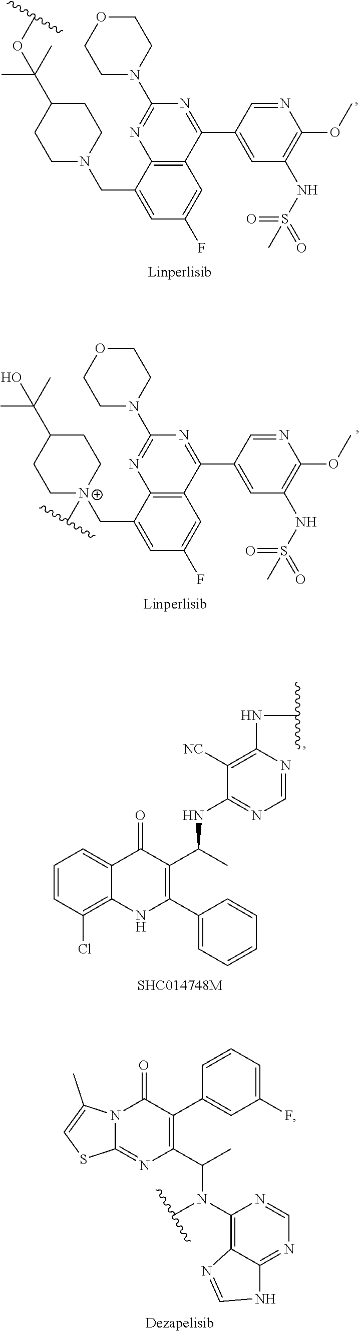 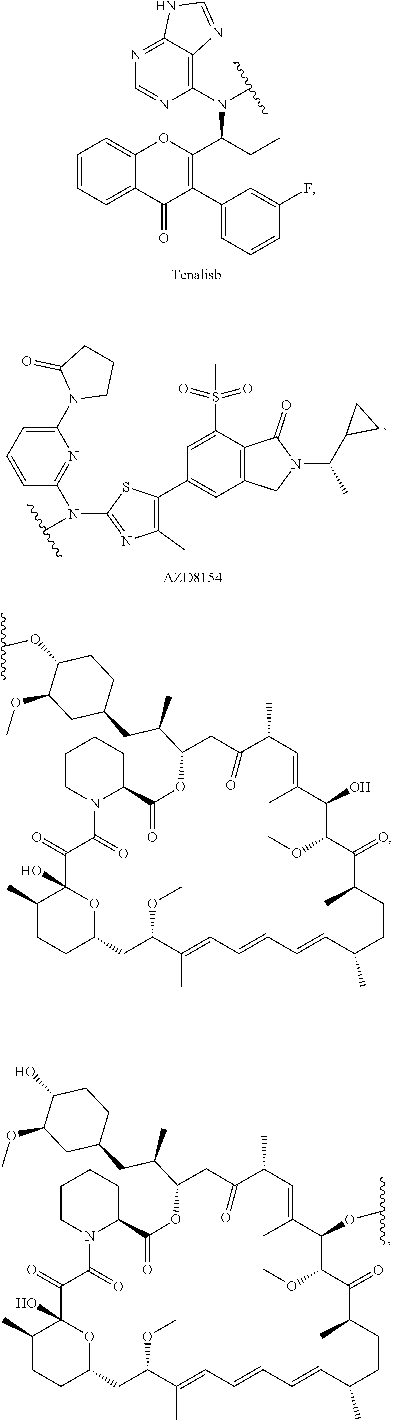 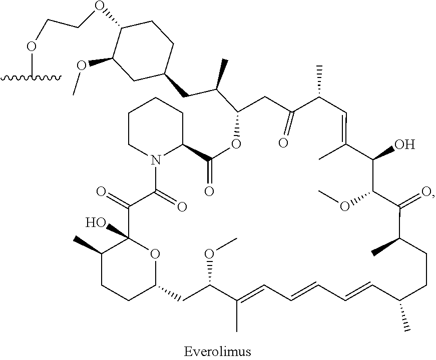 [SMILES: *OCCO[C@@H]1CC[C@@H](C[C@@H](C)[C@@H]2CC(=O)[C@H](C)/C=C(\C)[C@@H](O)[C@@H](OC)C(=O)[C@H](C)CC[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]
or hydrated salts; or the polymorphic crystalline structures of these compounds; or the optical isomers, racemates, diastereomers or enantiomers; [symbol] is a linkage site that links to L₁ and/or L₂ independently.

26. The antibody drug conjugate according to claim 1, wherein the cytotoxic drug, D₁ and D₂, are independently selected from, A Histone deacetylase inhibitor (HDACI), having the following formula: 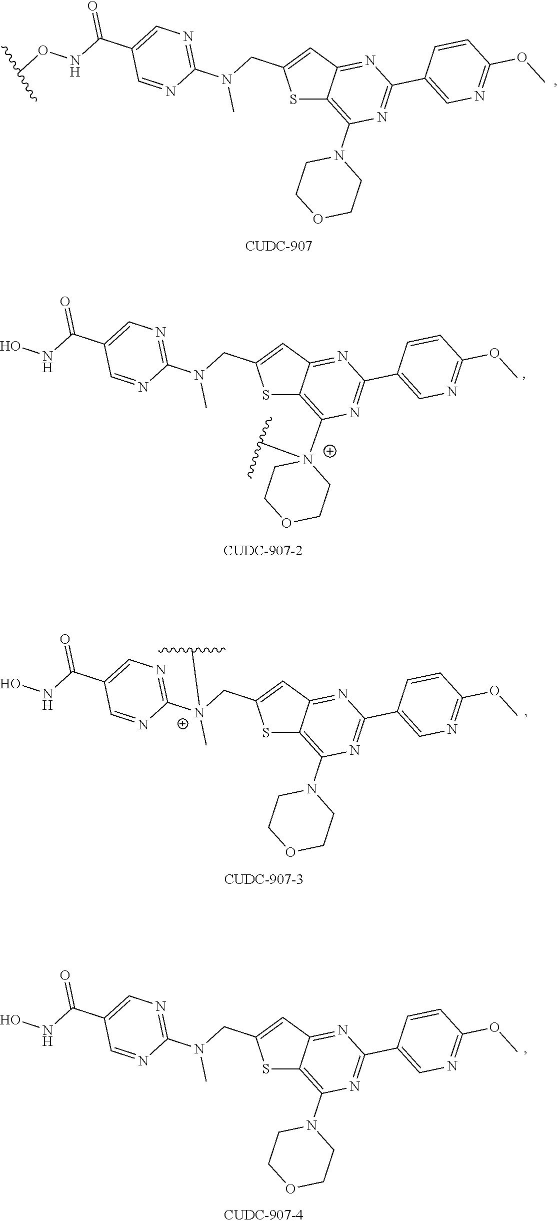 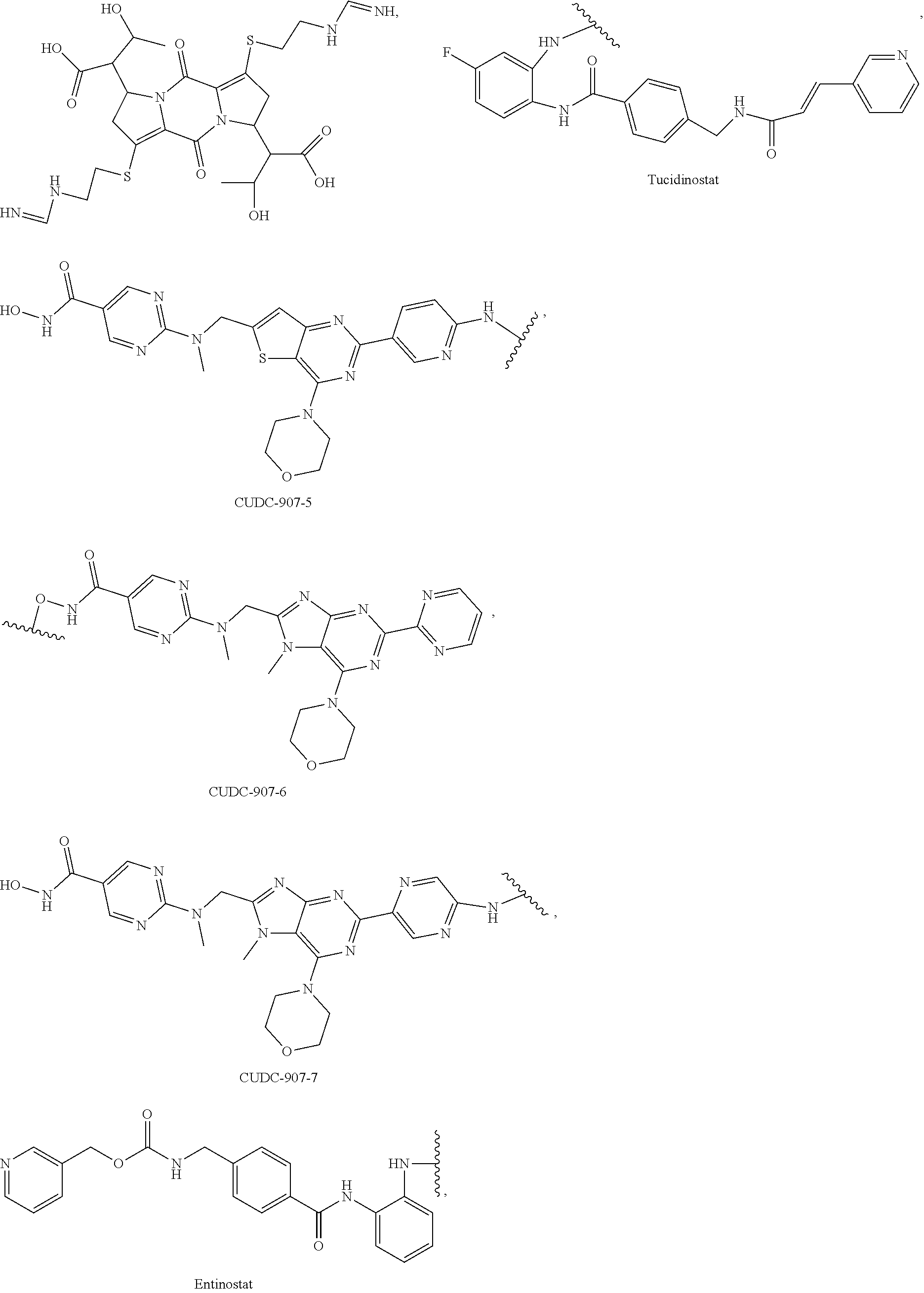 [SMILES: CC(O)C(C(=O)O)C1CC(SCCNC=N)=c2c(=O)n3c(c(=O)n21)=C(SCCNC=N)CC3C(C(=O)O)C(C)O ; *Nc1cc(F)ccc1NC(=O)c1ccc(CNC(=O)/C=C/c2cccnc2)cc1 ; *Nc1ccc(-c2nc(N3CCOCC3)c3sc(CN(C)c4ncc(C(=O)NO)cn4)cc3n2)cn1 ; *ONC(=O)c1cnc(N(C)Cc2nc3nc(-c4ncccn4)nc(N4CCOCC4)c3n2C)nc1 ; *Nc1cnc(-c2nc(N3CCOCC3)c3c(n2)nc(CN(C)c2ncc(C(=O)NO)cn2)n3C)cn1 ; *Nc1ccccc1NC(=O)c1ccc(CNC(=O)OCc2cccnc2)cc1] 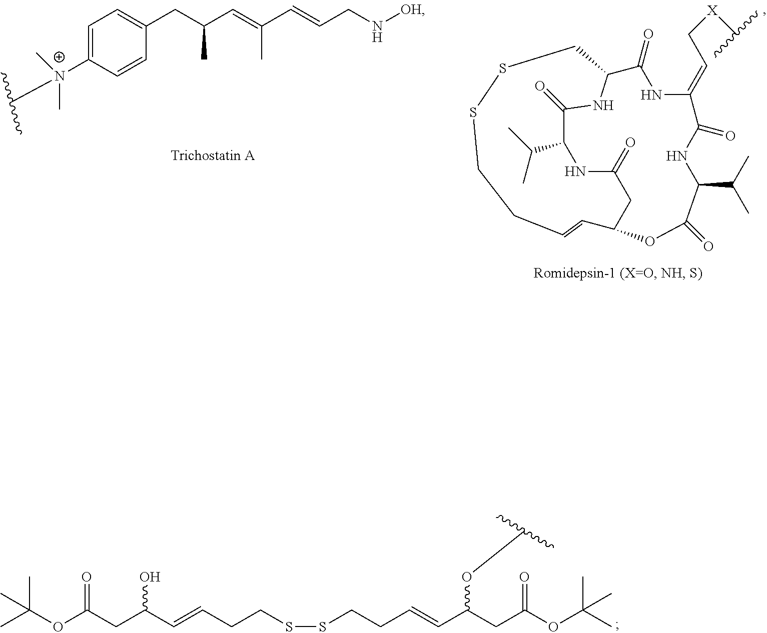
or hydrated salts; or the polymorphic crystalline structures of these compounds; or the optical isomers, racemates, diastereomers or enantiomers; [symbol] is a linkage site that links to L₁ and/or L₂ independently.

27. The antibody drug conjugate according to claim 1, wherein the cytotoxic drug, D₁ and D₂, are independently selected from, an Inhibitor of Nuclear factor kappa B (NF-κB), having the following formula: 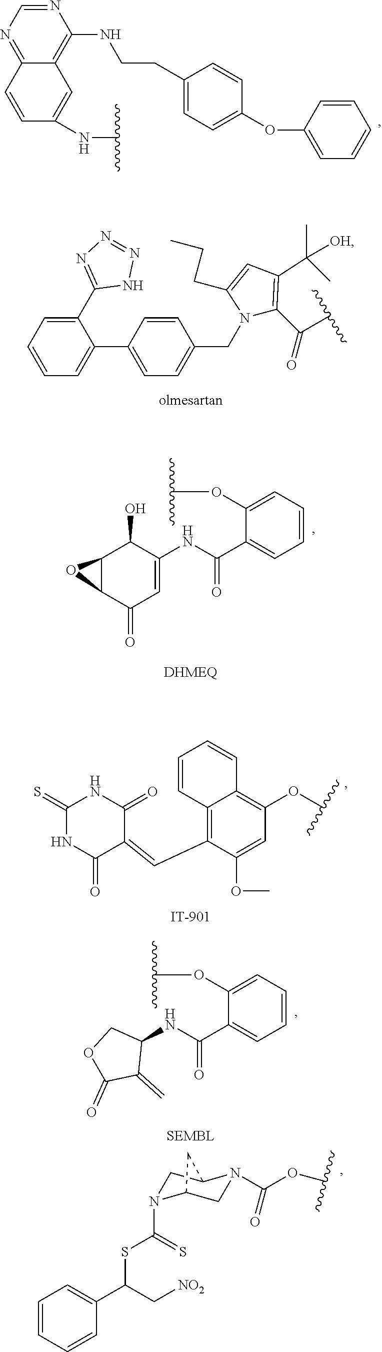 [SMILES: *Nc1ccc2ncnc(NCCc3ccc(Oc4ccccc4)cc3)c2c1 ; *C(=O)c1c(C(C)(C)O)cc(CCC)n1Cc1ccc(-c2ccccc2-c2nnn[nH]2)cc1 ; *Oc1ccccc1C(=O)NC1=CC(=O)[C@H]2O[C@H]2[C@H]1O ; *Oc1cc(OC)c(C=C2C(=O)NC(=S)NC2=O)c2ccccc12 ; *Oc1ccccc1C(=O)N[C@@H]1COC(=O)C1=C ; *OC(=O)N1C[C@H]2C[C@@H]1CN2C(=S)SC(C[N+](=O)[O-])c1ccccc1] 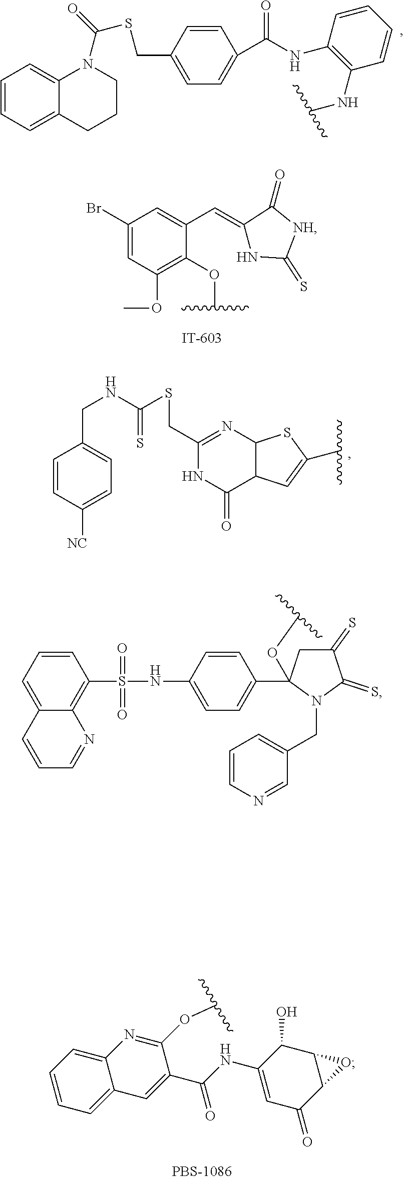 [SMILES: *Nc1ccccc1NC(=O)c1ccc(CSC(=O)N2CCCc3ccccc32)cc1 ; *Oc1c(/C=C2\NC(=S)NC2=O)cc(Br)cc1OC ; *C1=CC2C(=O)NC(CSC(=S)NCc3ccc(C#N)cc3)=NC2S1 ; *OC1(c2ccc(NS(=O)(=O)c3cccc4cccnc34)cc2)CC(=S)C(=S)N1Cc1cccnc1 ; *Oc1nc2ccccc2cc1C(=O)NC1=CC(=O)[C@H]2O[C@H]2[C@H]1O]
or an isotope of one or more chemical elements, or pharmaceutically acceptable salts, hydrates, or hydrated salts; or the polymorphic crystalline structures of these compounds; or the optical isomers, racemates, diastereomers or enantiomers; [symbol] is a linkage site that links to L₁ and/or L₂ independently.

28. The antibody drug conjugate according to claim 1, wherein the cytotoxic drug, D₁ and D₂, are independently selected from, a PD-L1/PD-1 ligand, having the following formula: 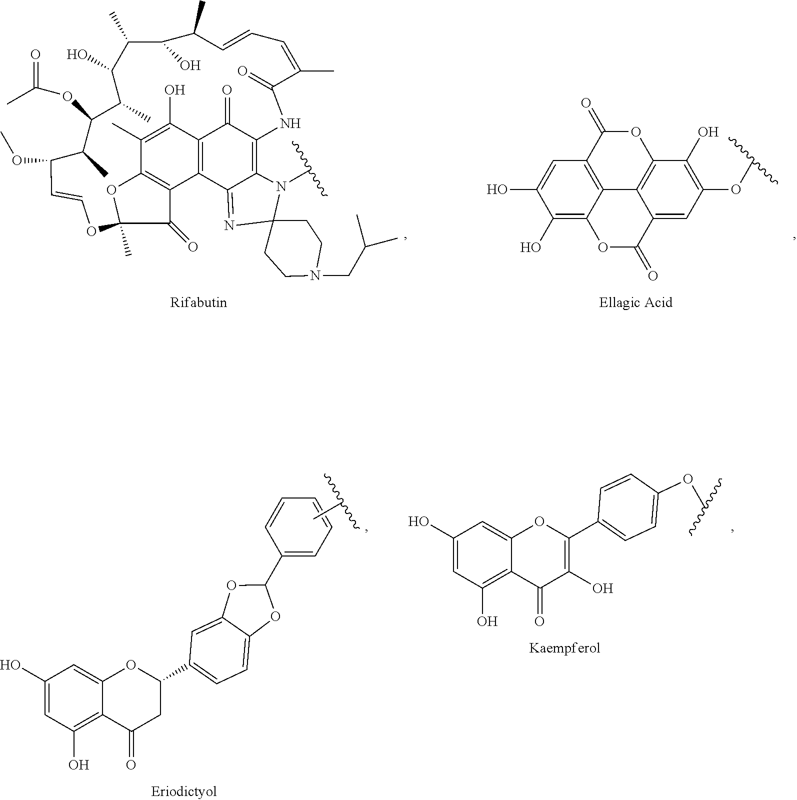 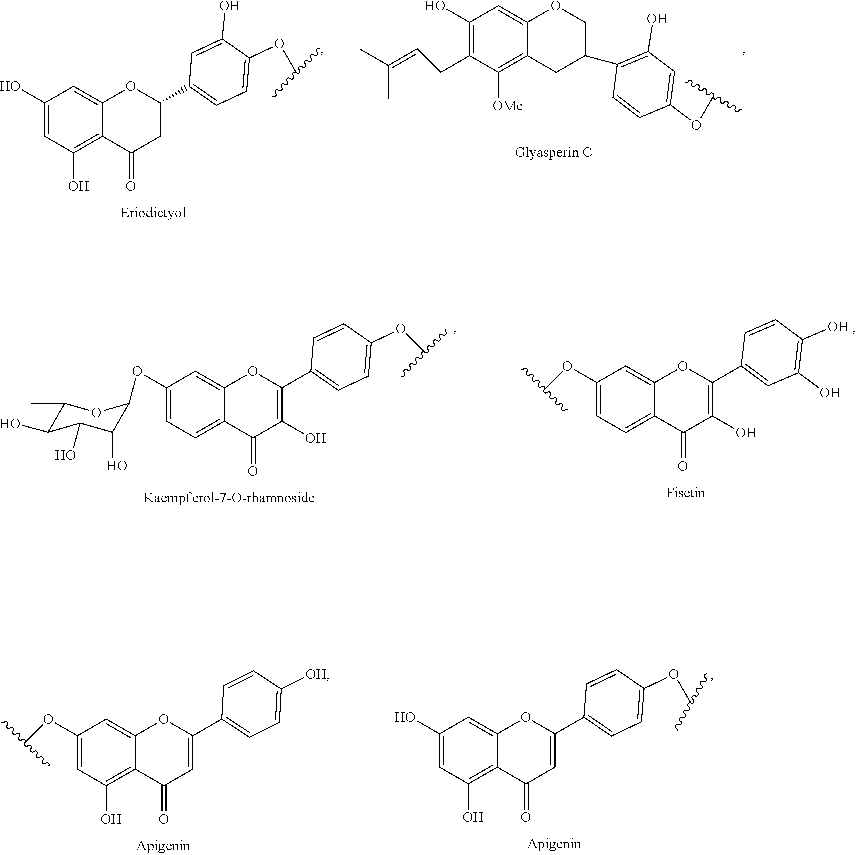 [SMILES: *Oc1ccc([C@@H]2CC(=O)c3c(O)cc(O)cc3O2)cc1O ; *Oc1ccc(C2COc3cc(O)c(CC=C(C)C)c(OC)c3C2)c(O)c1 ; *Oc1ccc(-c2oc3cc(OC4OC(C)C(O)C(O)C4O)ccc3c(=O)c2O)cc1 ; *Oc1ccc2c(=O)c(O)c(-c3ccc(O)c(O)c3)oc2c1 ; *Oc1cc(O)c2c(=O)cc(-c3ccc(O)cc3)oc2c1 ; *Oc1ccc(-c2cc(=O)c3c(O)cc(O)cc3o2)cc1] 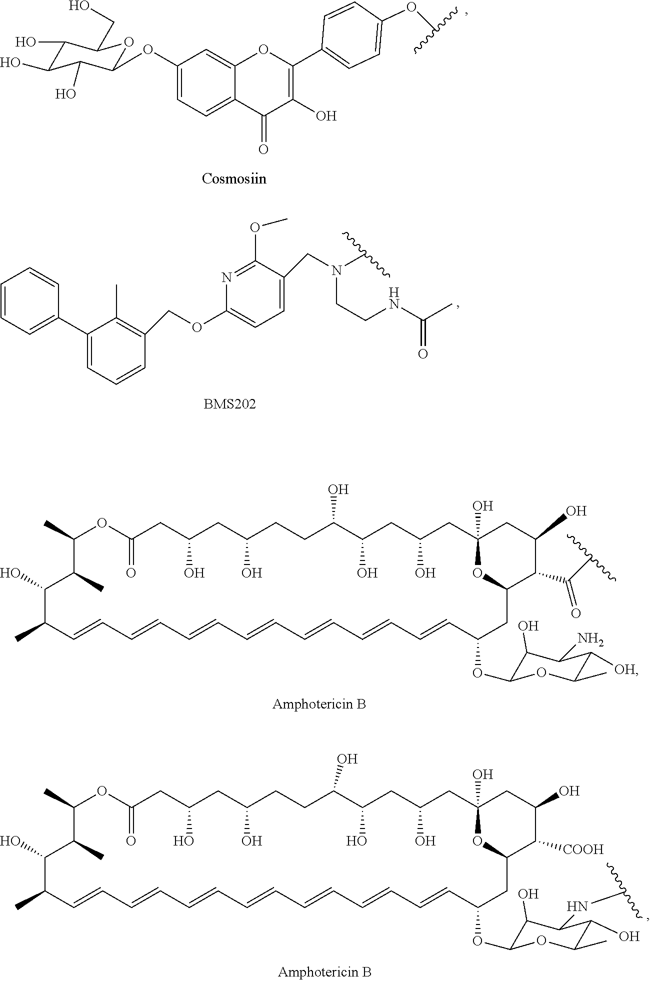 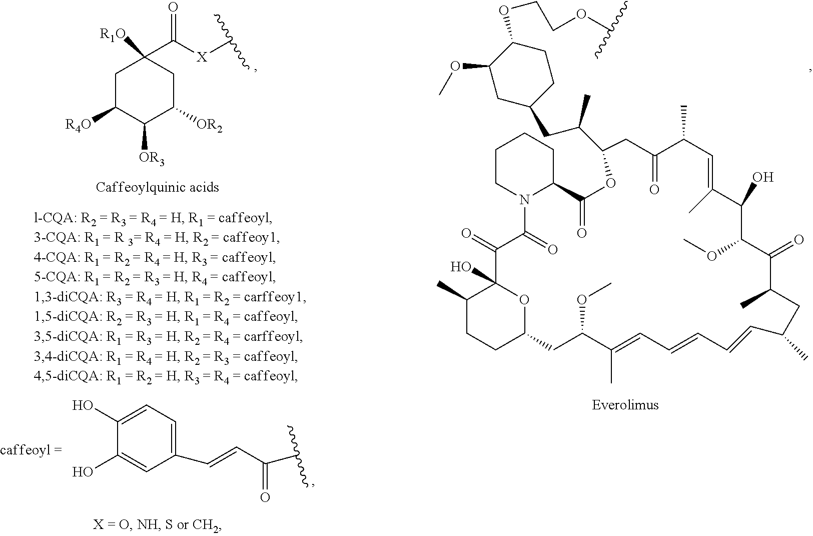 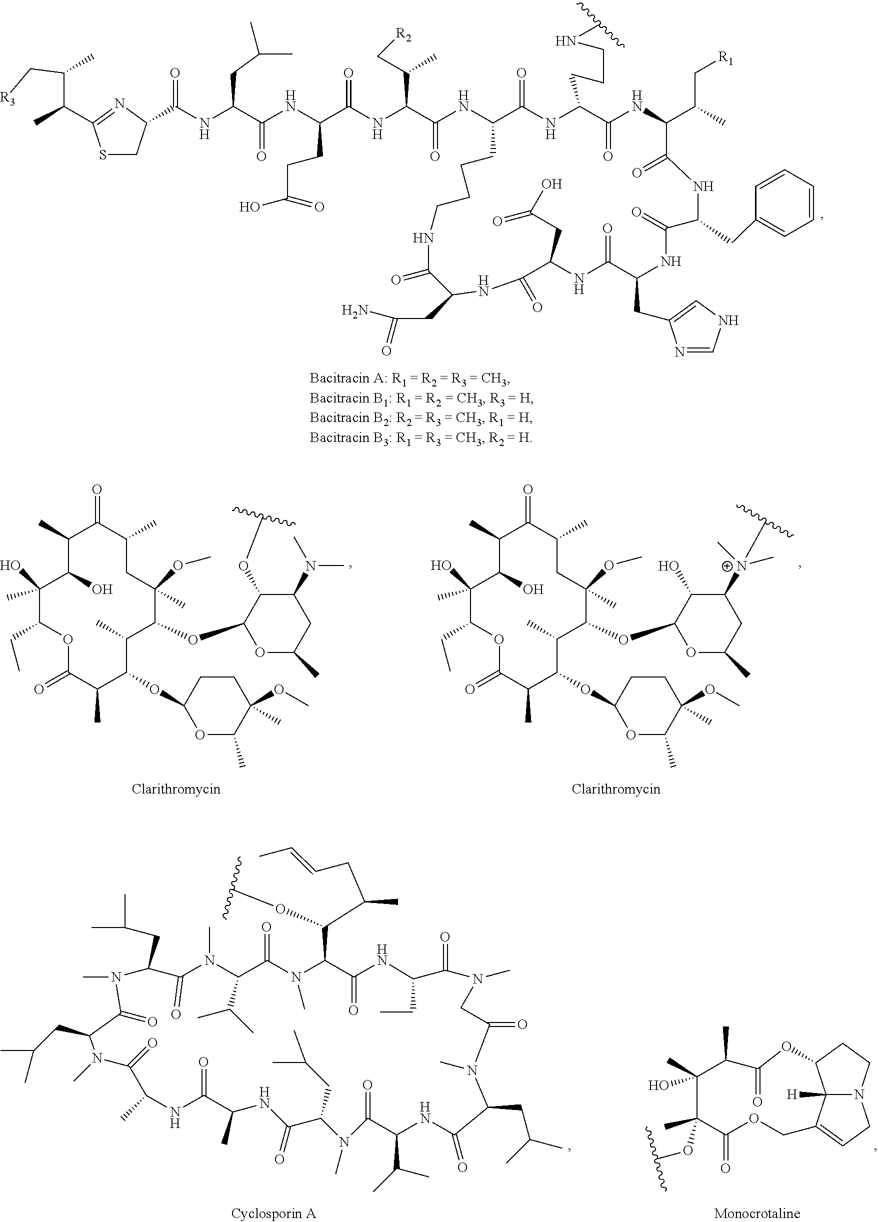 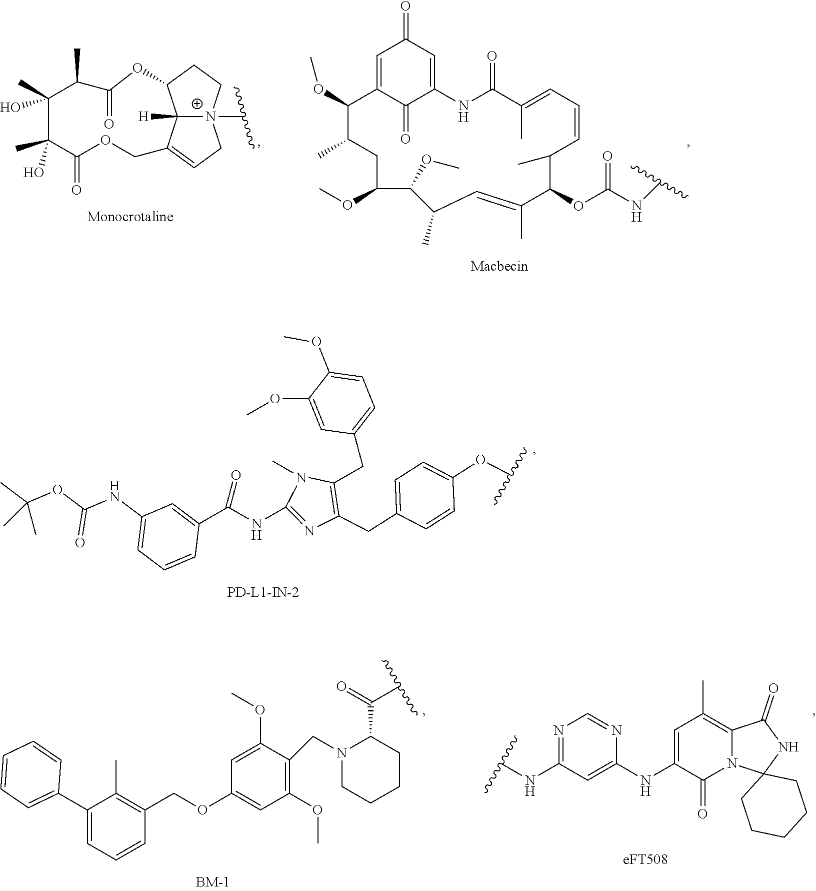 [SMILES: *[N+]12CC=C3COC(=O)[C@](C)(O)[C@](C)(O)[C@@H](C)C(=O)O[C@H](CC1)[C@@H]32 ; *NC(=O)O[C@H]1/C(C)=C/[C@H](C)[C@@H](OC)[C@@H](OC)C[C@H](C)[C@@H](OC)C2=CC(=O)C=C(NC(=O)/C(C)=C/C=C\C1C)C2=O ; *Oc1ccc(Cc2nc(NC(=O)c3cccc(NC(=O)OC(C)(C)C)c3)n(C)c2Cc2ccc(OC)c(OC)c2)cc1 ; *C(=O)[C@@H]1CCCCN1Cc1c(OC)cc(OCc2cccc(-c3ccccc3)c2C)cc1OC ; *Nc1cc(Nc2cc(C)c3n(c2=O)C2(CCCCC2)NC3=O)ncn1] 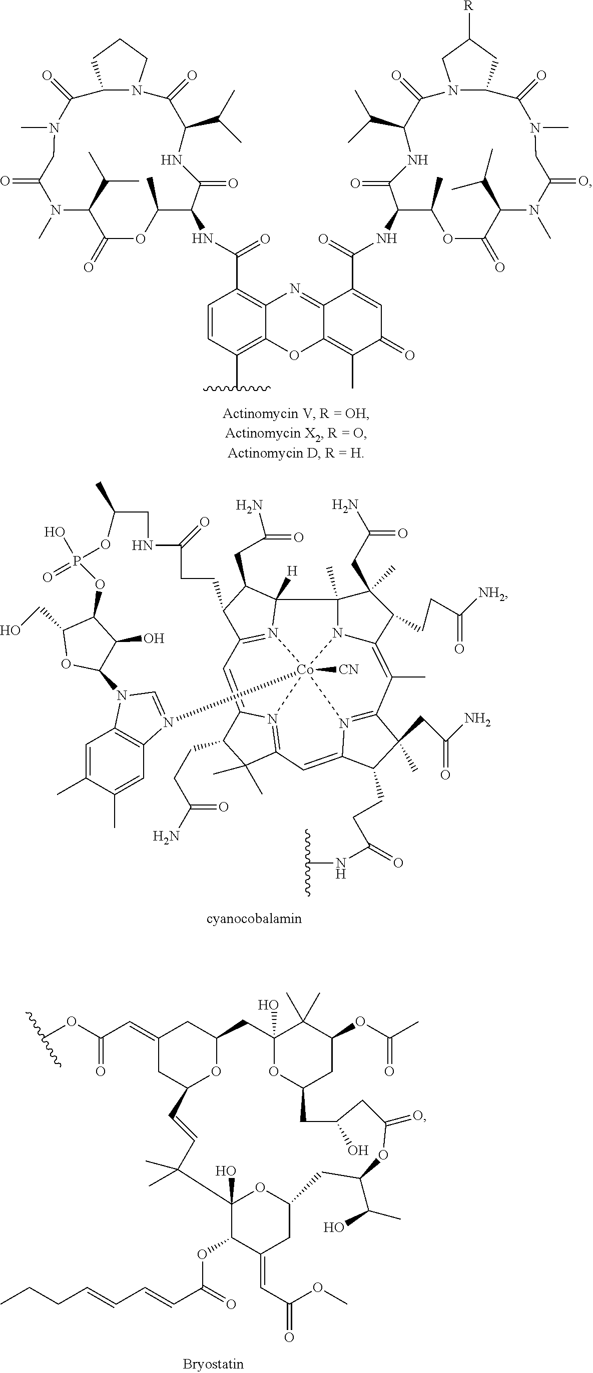 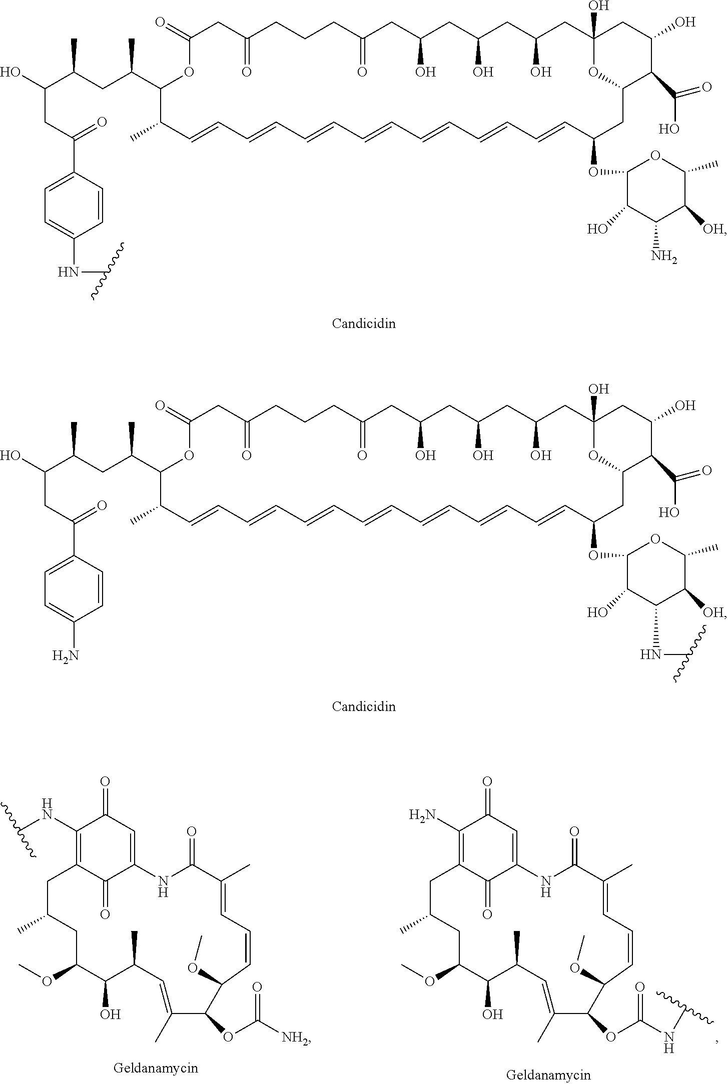 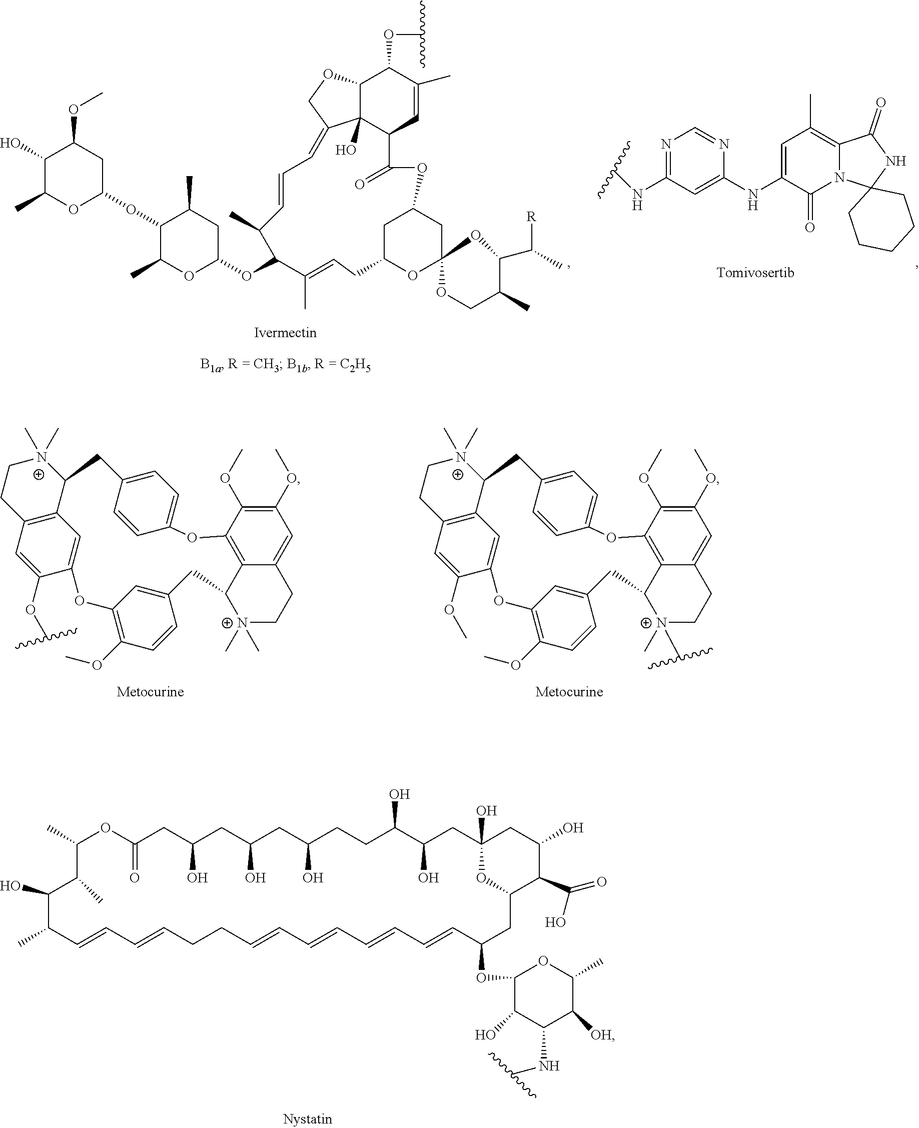 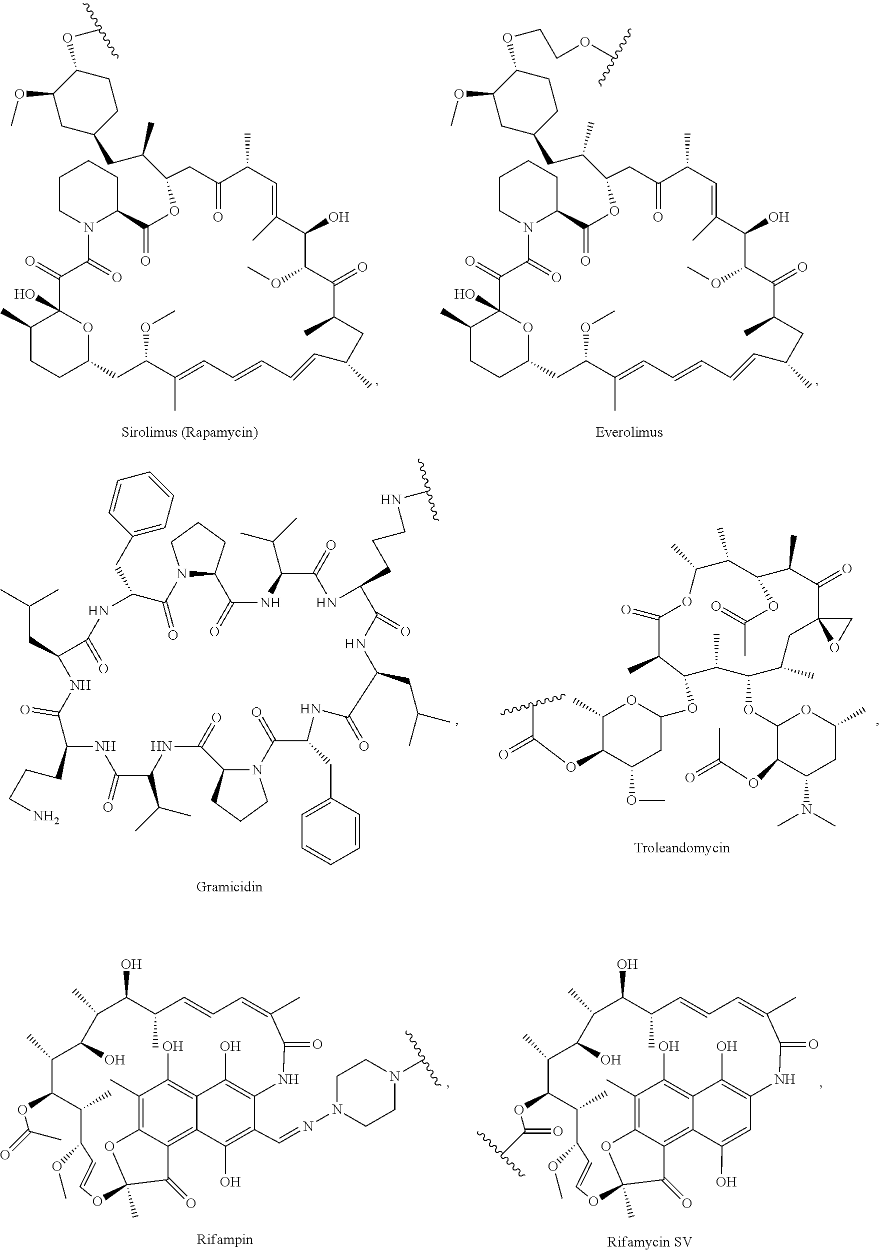 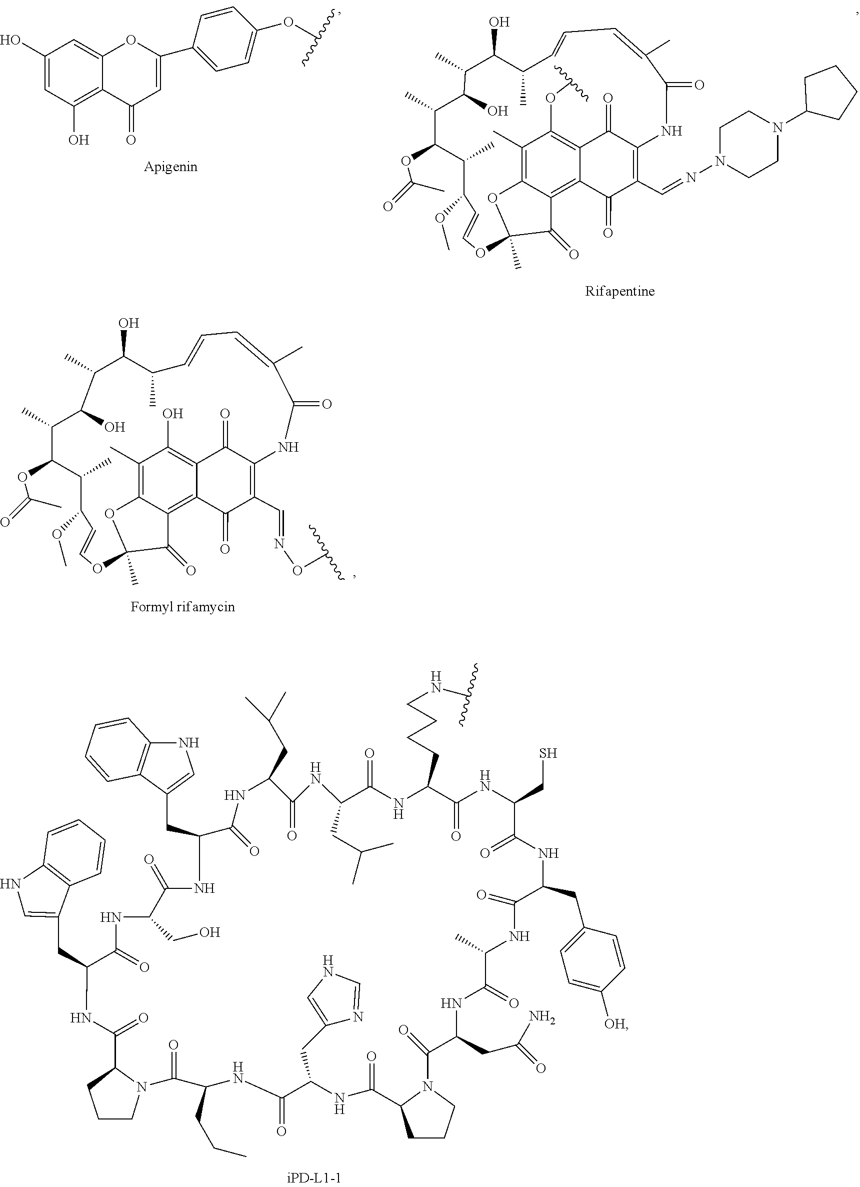 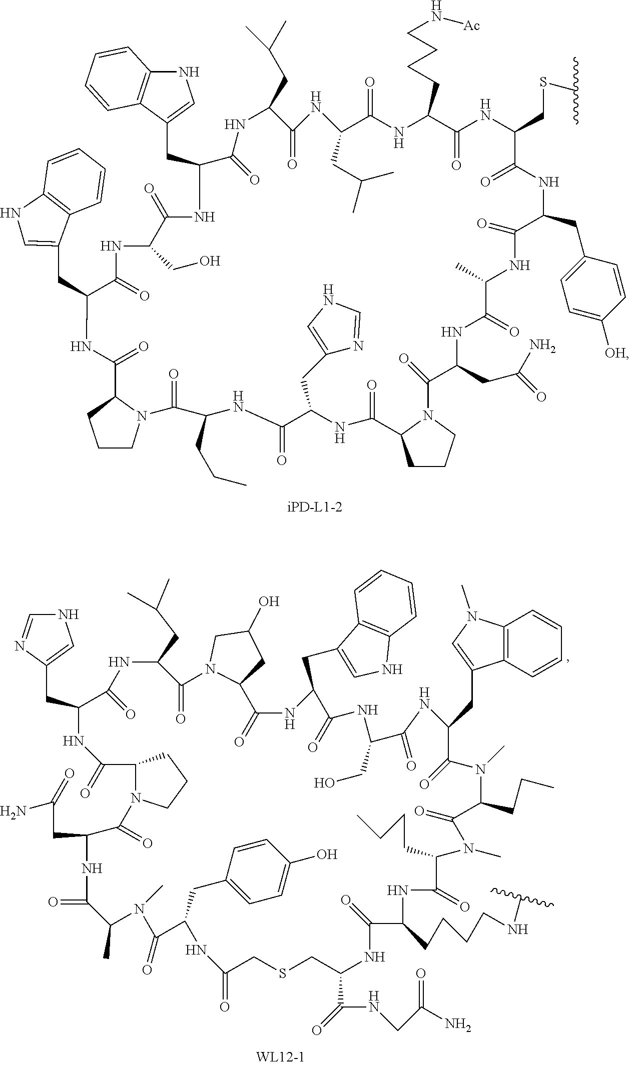 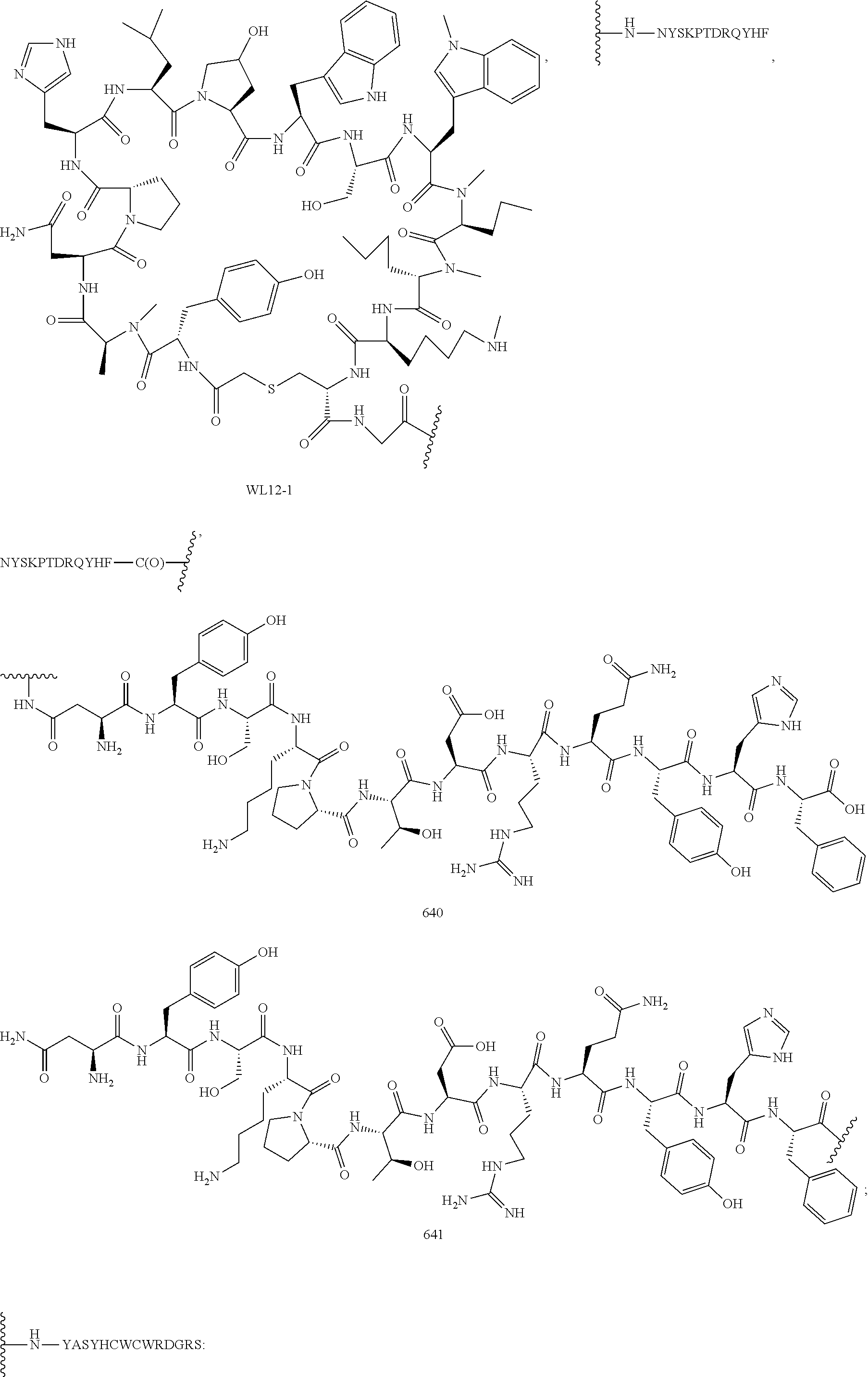 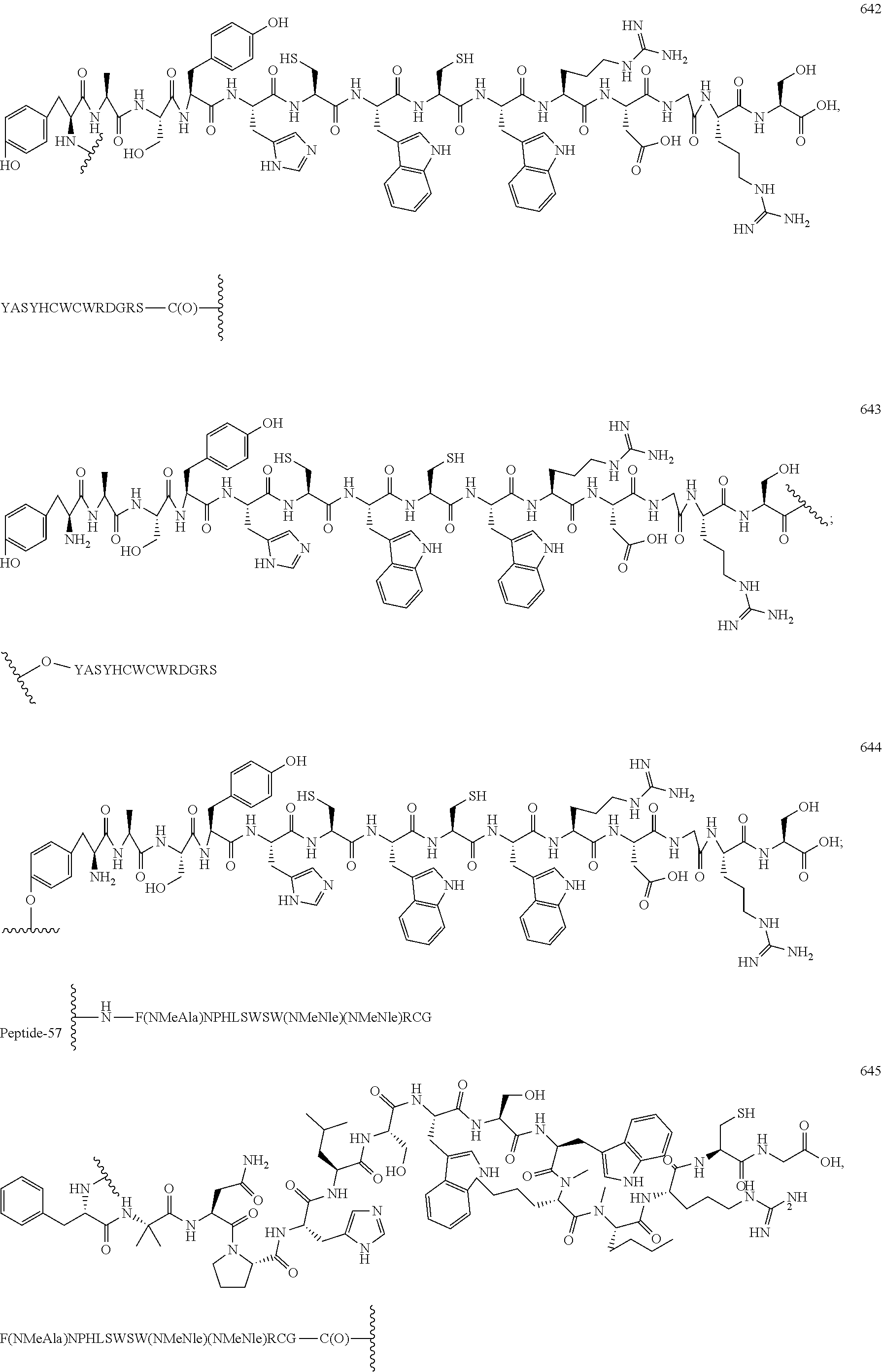 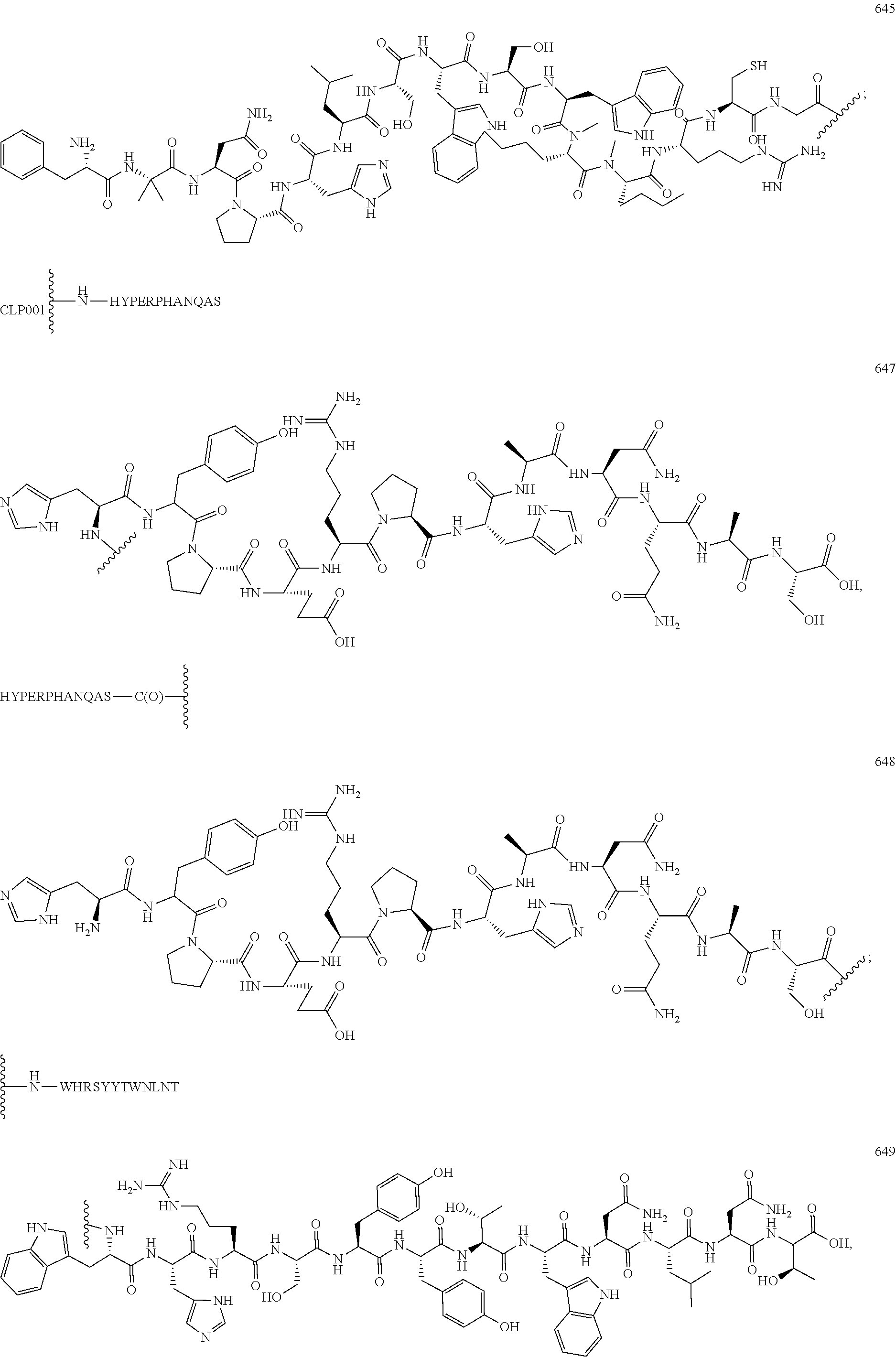 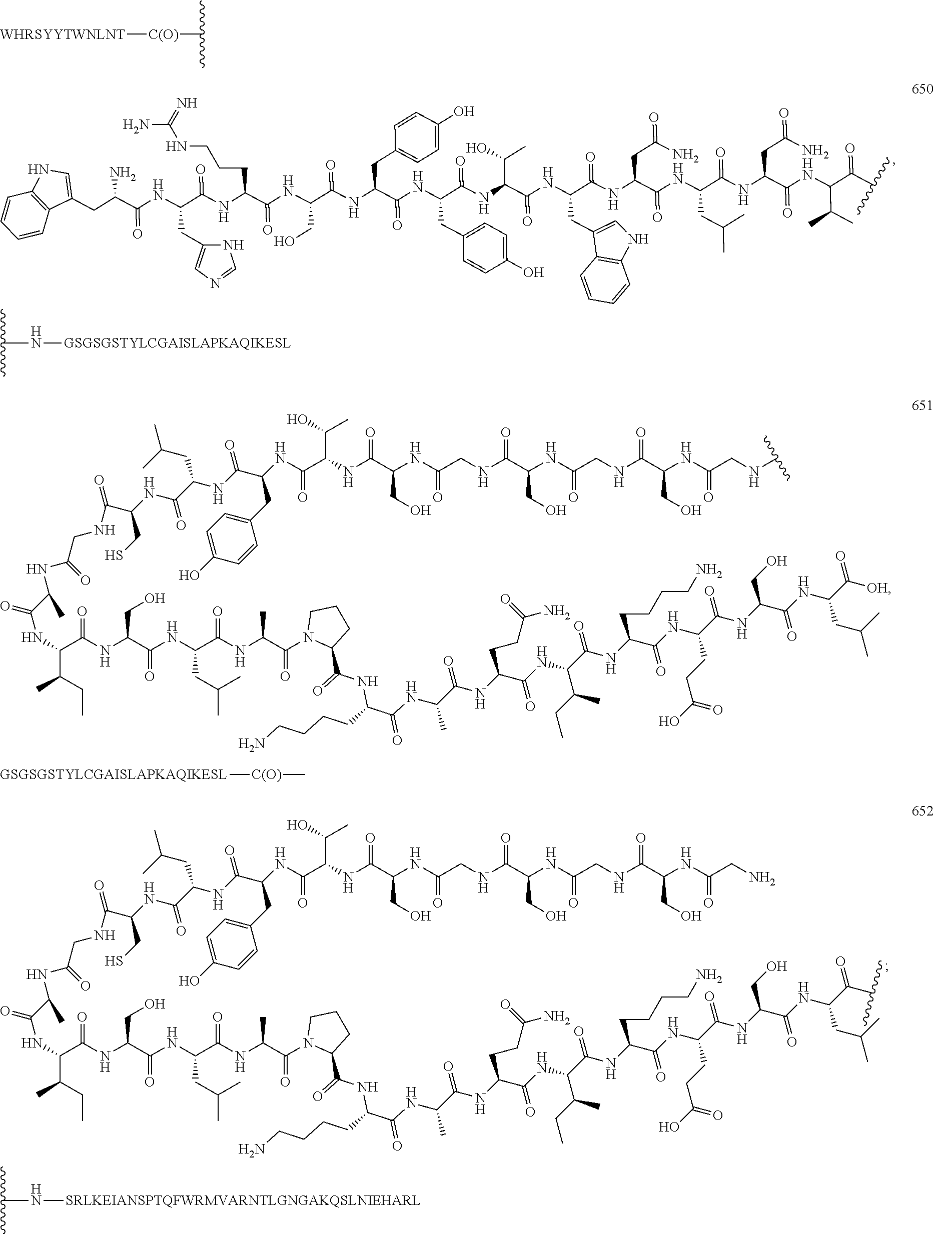 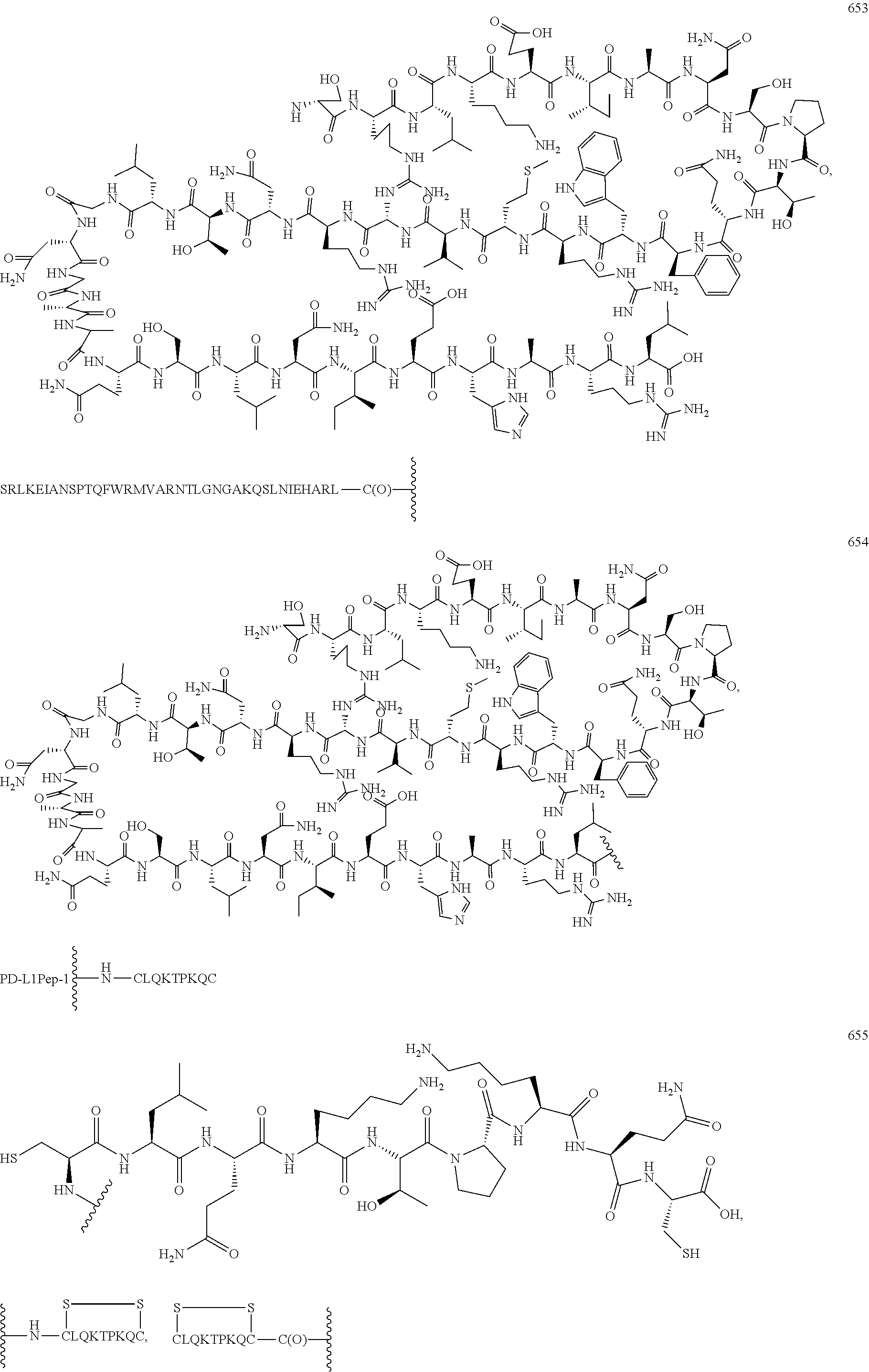 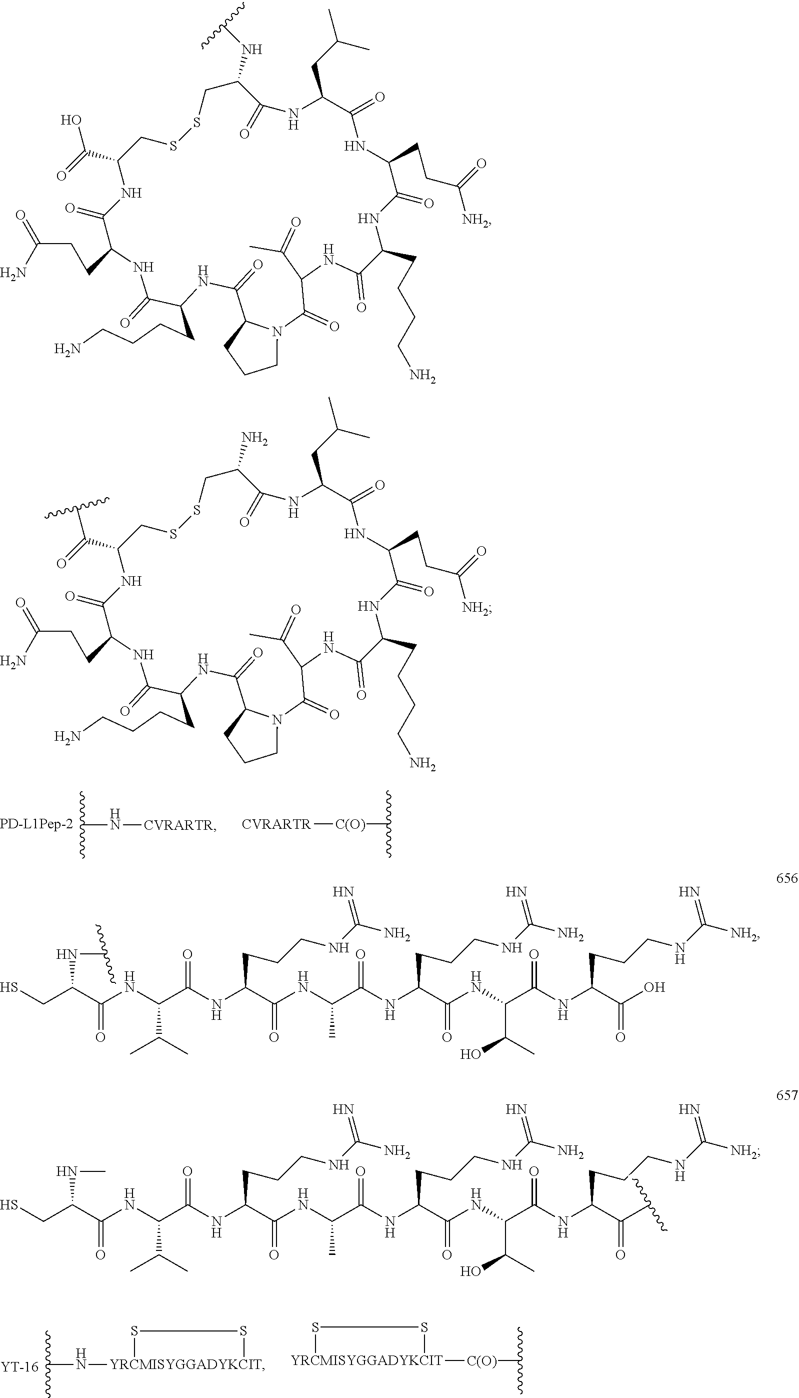 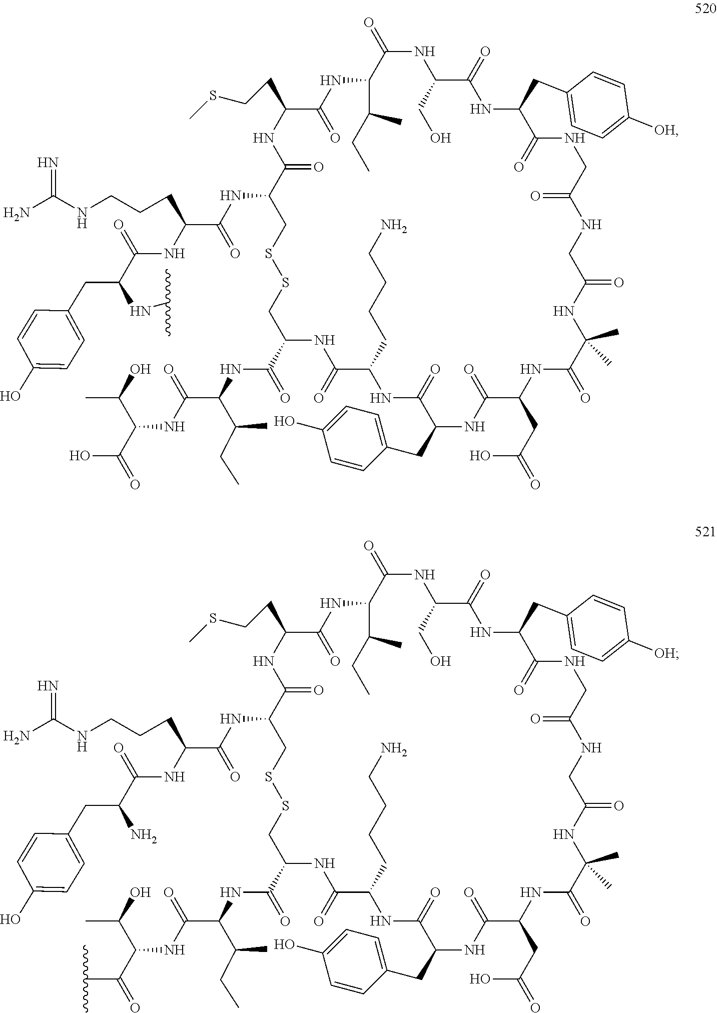
or hydrated salts; or the polymorphic crystalline structures of these compounds; or the optical isomers, racemates, diastereomers or enantiomers; [symbol] is a linkage site that links to L₁ and/or L₂ independently.

29. The antibody drug conjugate according to claim 1, wherein the cytotoxic drug, D₁ and D₂, are independently selected from, an Inhibitor of Kinesin spindle proteins (KSP), having the following formula: 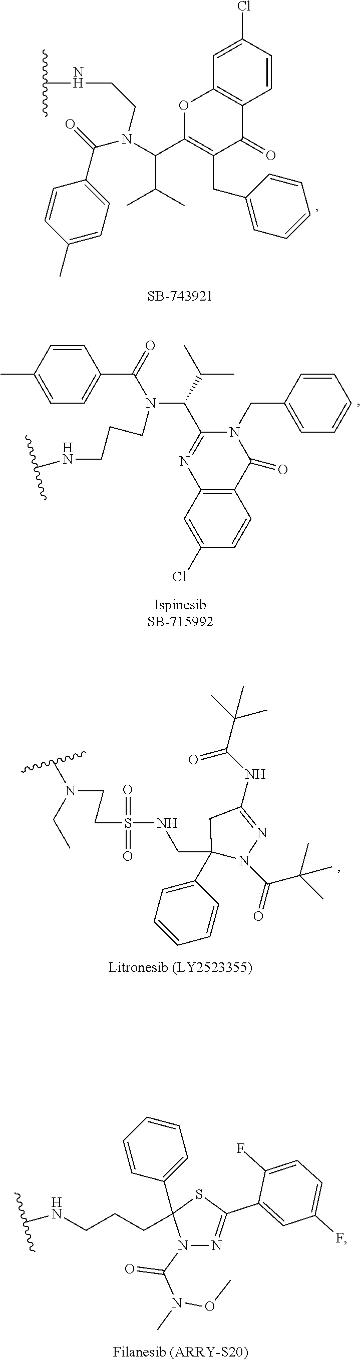 [SMILES: *NCCN(C(=O)c1ccc(C)cc1)C(c1oc2cc(Cl)ccc2c(=O)c1Cc1ccccc1)C(C)C ; *NCCCN(C(=O)c1ccc(C)cc1)[C@@H](c1nc2cc(Cl)ccc2c(=O)n1Cc1ccccc1)C(C)C ; *N(CC)CCS(=O)(=O)NCC1(c2ccccc2)CC(NC(=O)C(C)(C)C)=NN1C(=O)C(C)(C)C ; *NCCCC1(c2ccccc2)SC(c2cc(F)ccc2F)=NN1C(=O)N(C)OC] 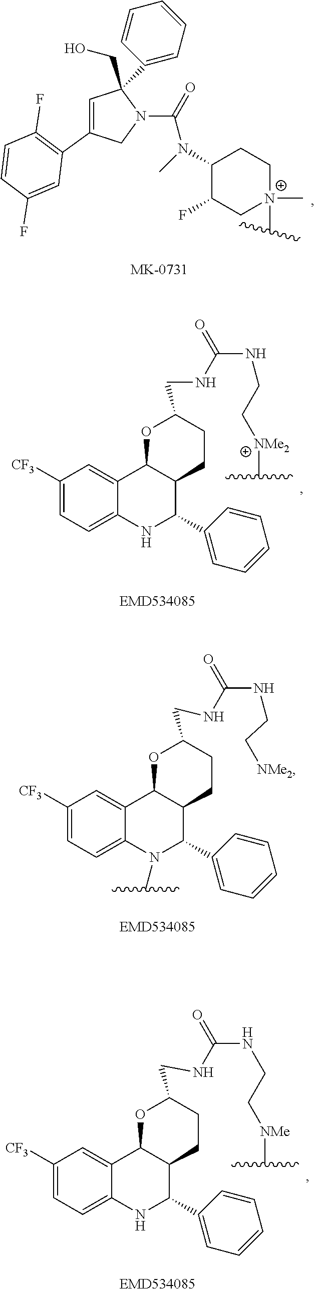 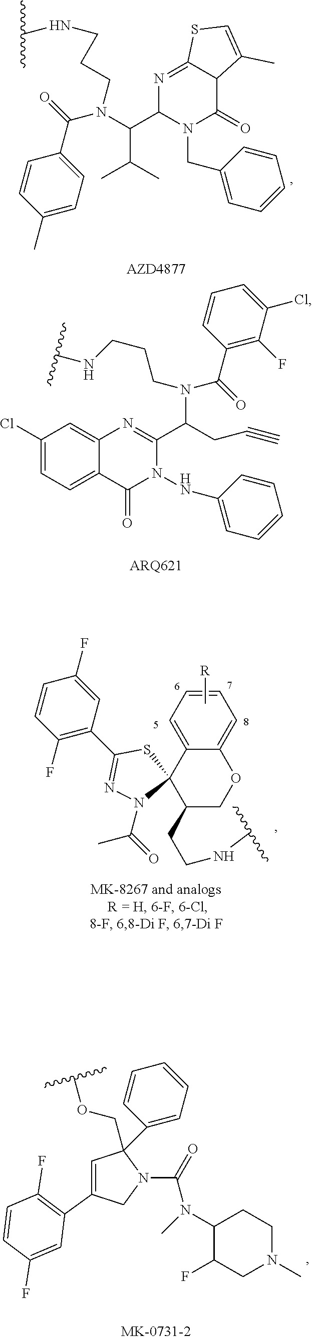
or an isotope of one or more chemical elements, or pharmaceutically acceptable salts, hydrates, or hydrated salts; or the polymorphic crystalline structures of these compounds; or the optical isomers, racemates, diastereomers or enantiomers; [symbol] is a linkage site that links to L₁ and/or L₂ independently.

30. The antibody drug conjugate according to claim 1, wherein the cytotoxic drug, D₁ and D₂, are independently selected from, An antiestrogen, having the following formula: 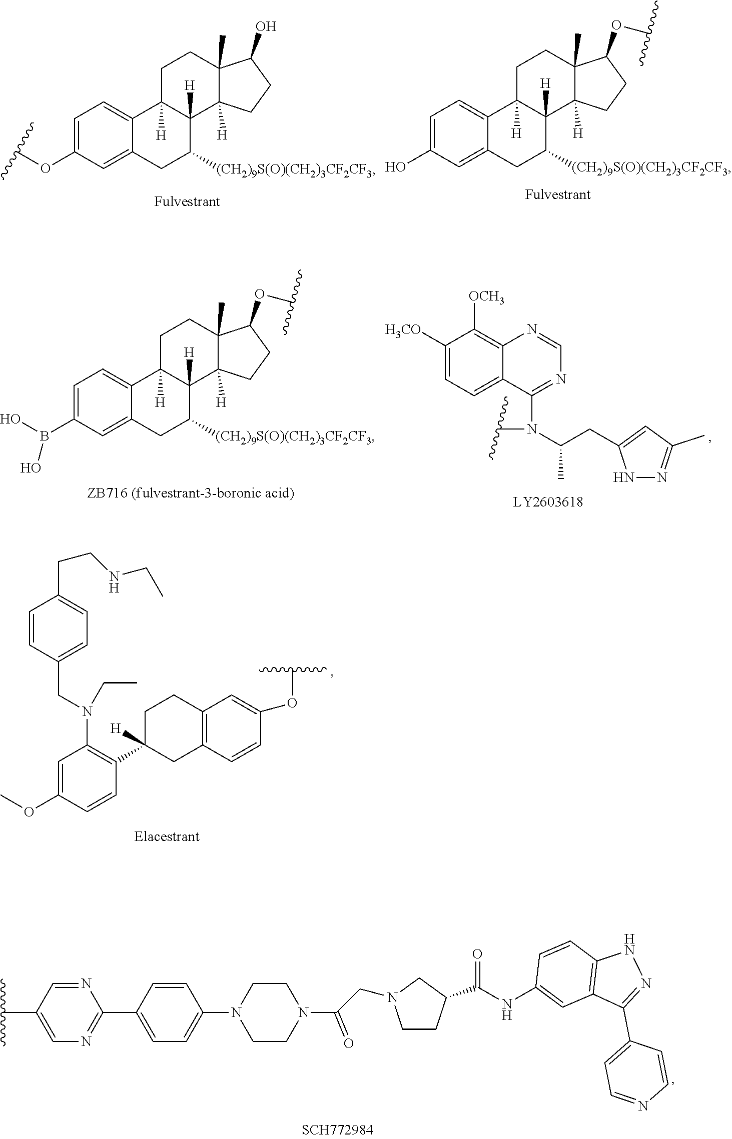 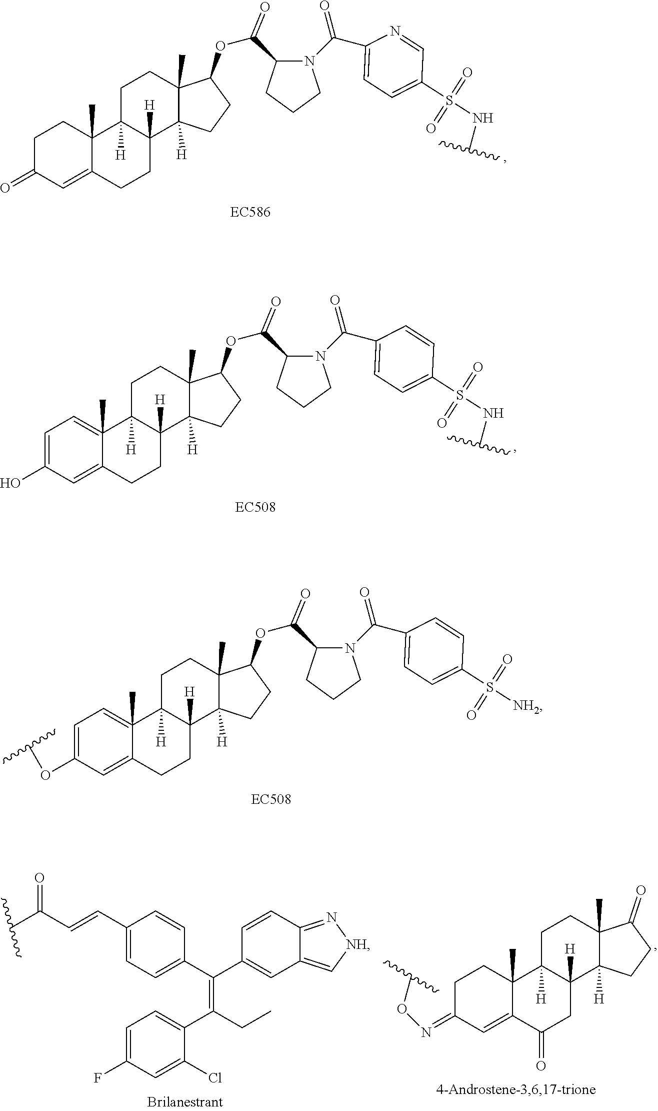 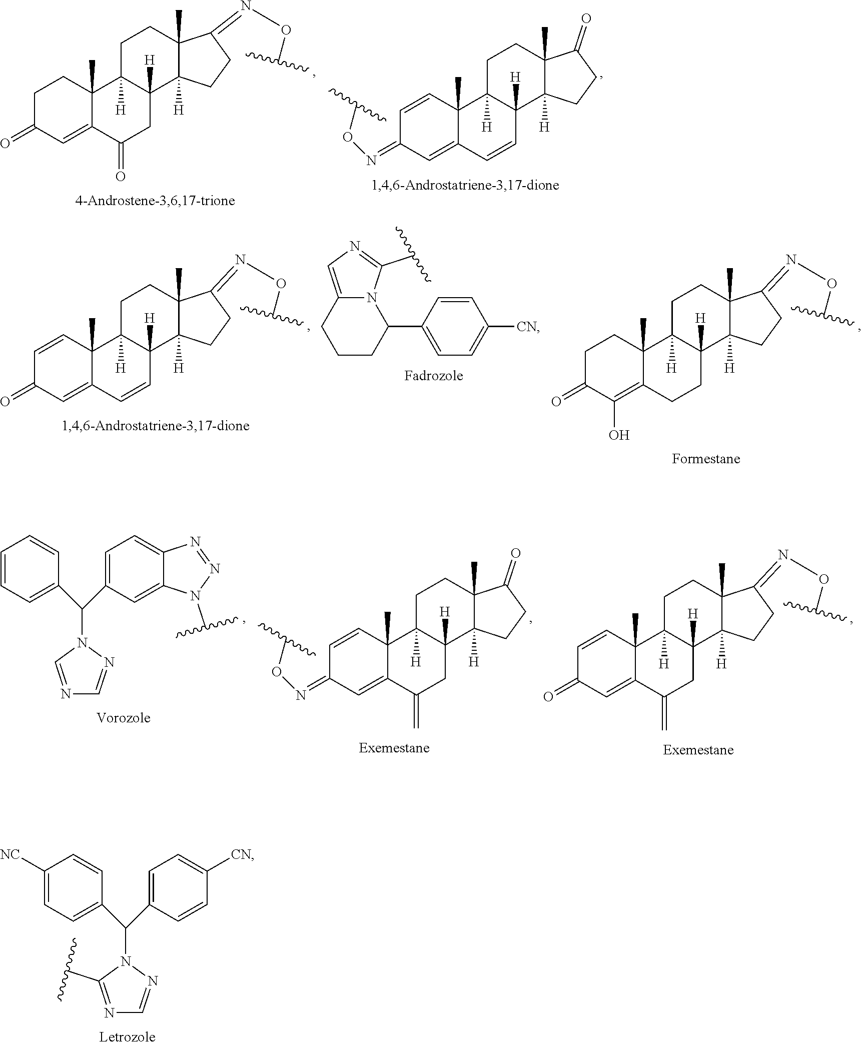
or hydrated salts; or the polymorphic crystalline structures of these compounds; or the optical isomers, racemates, diastereomers or enantiomers; [symbol] is a linkage site that links to L₁ and/or L₂ independently.

31. The antibody drug conjugate according to claim 1, wherein the cytotoxic drug, D₁ and D₂, are independently selected from, A Proteasome inhibitor (PI), having the following formula: 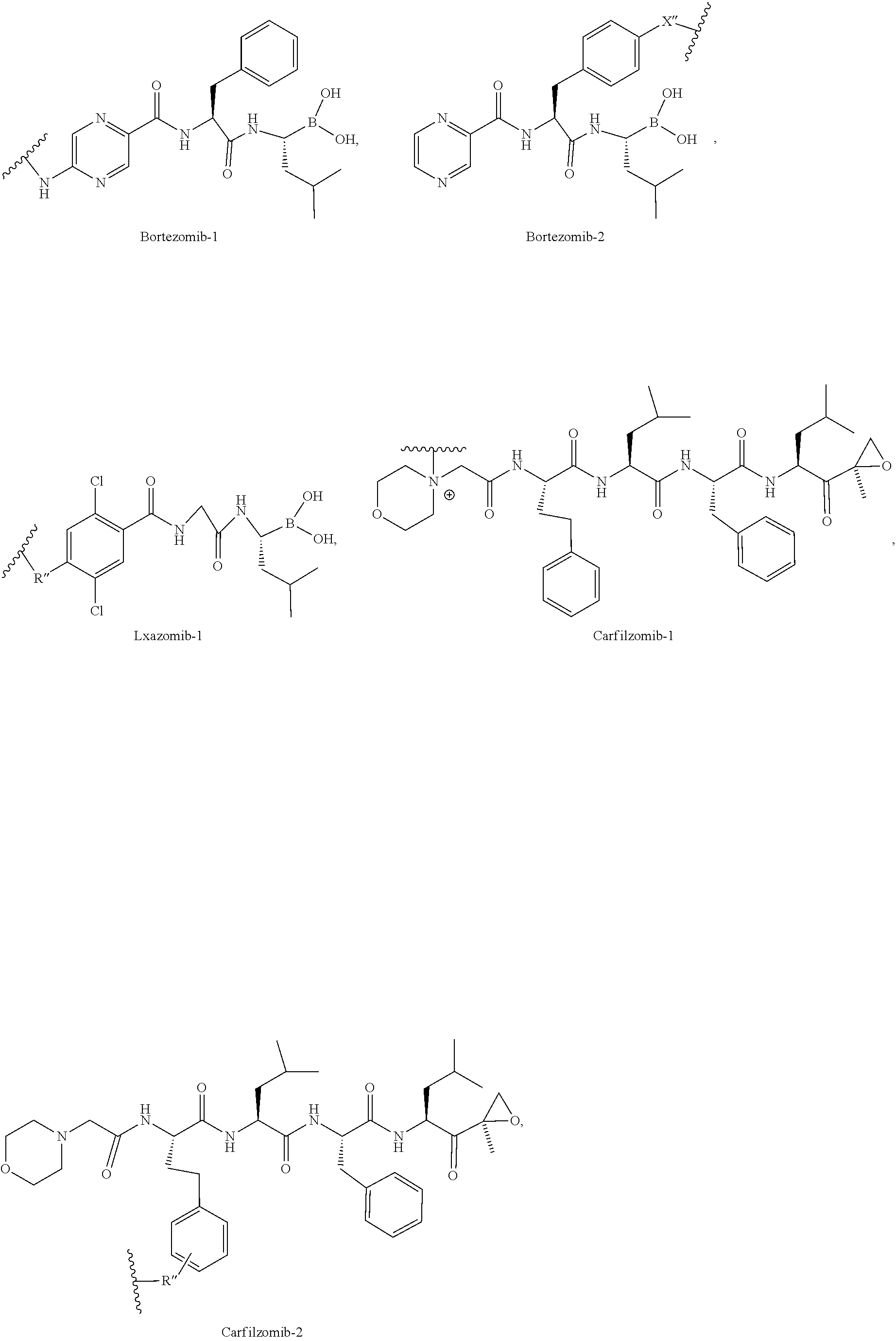 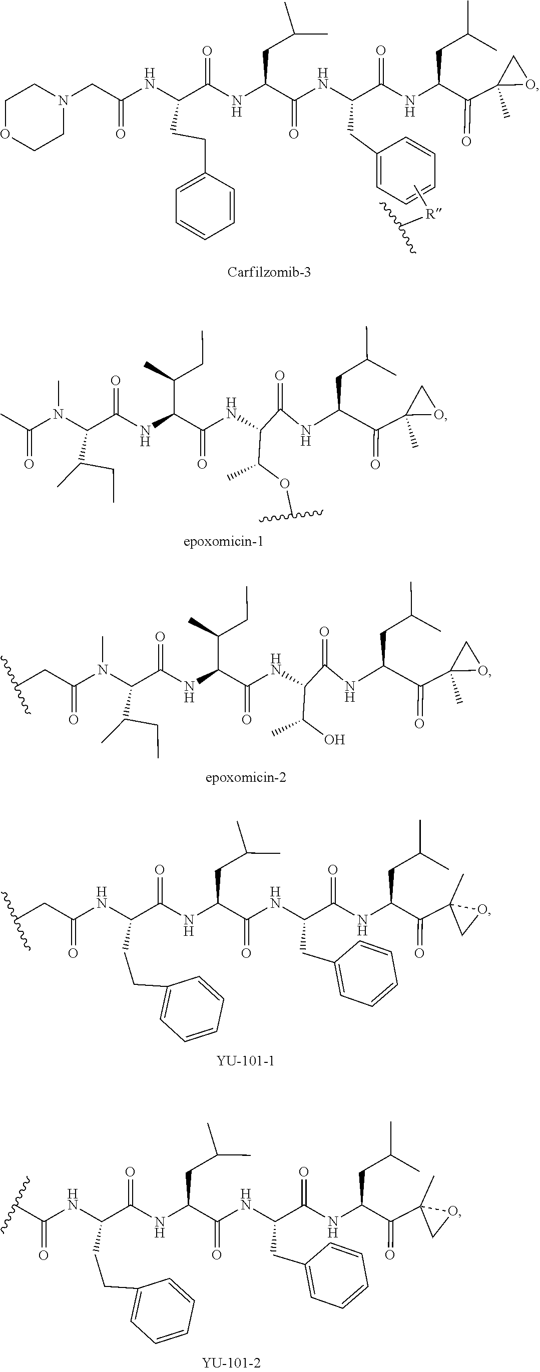 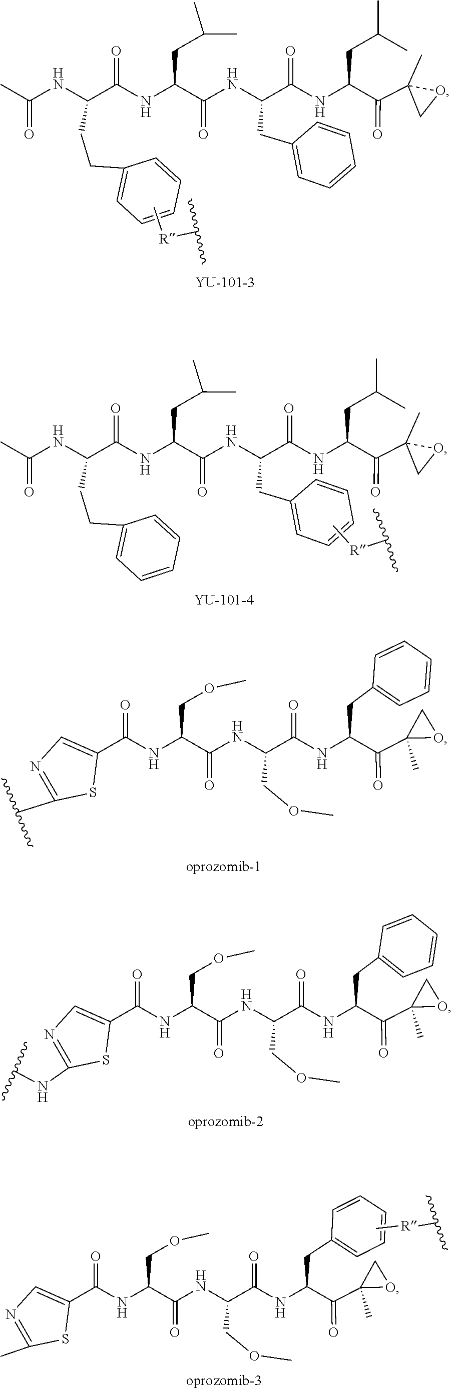 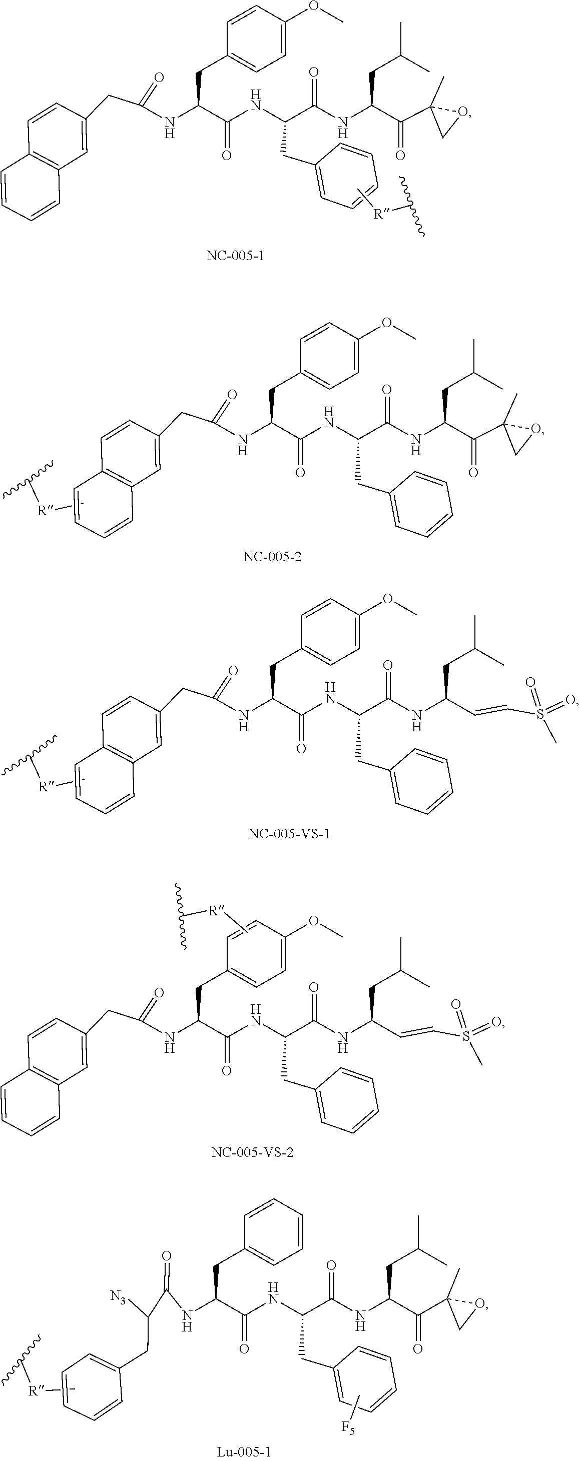 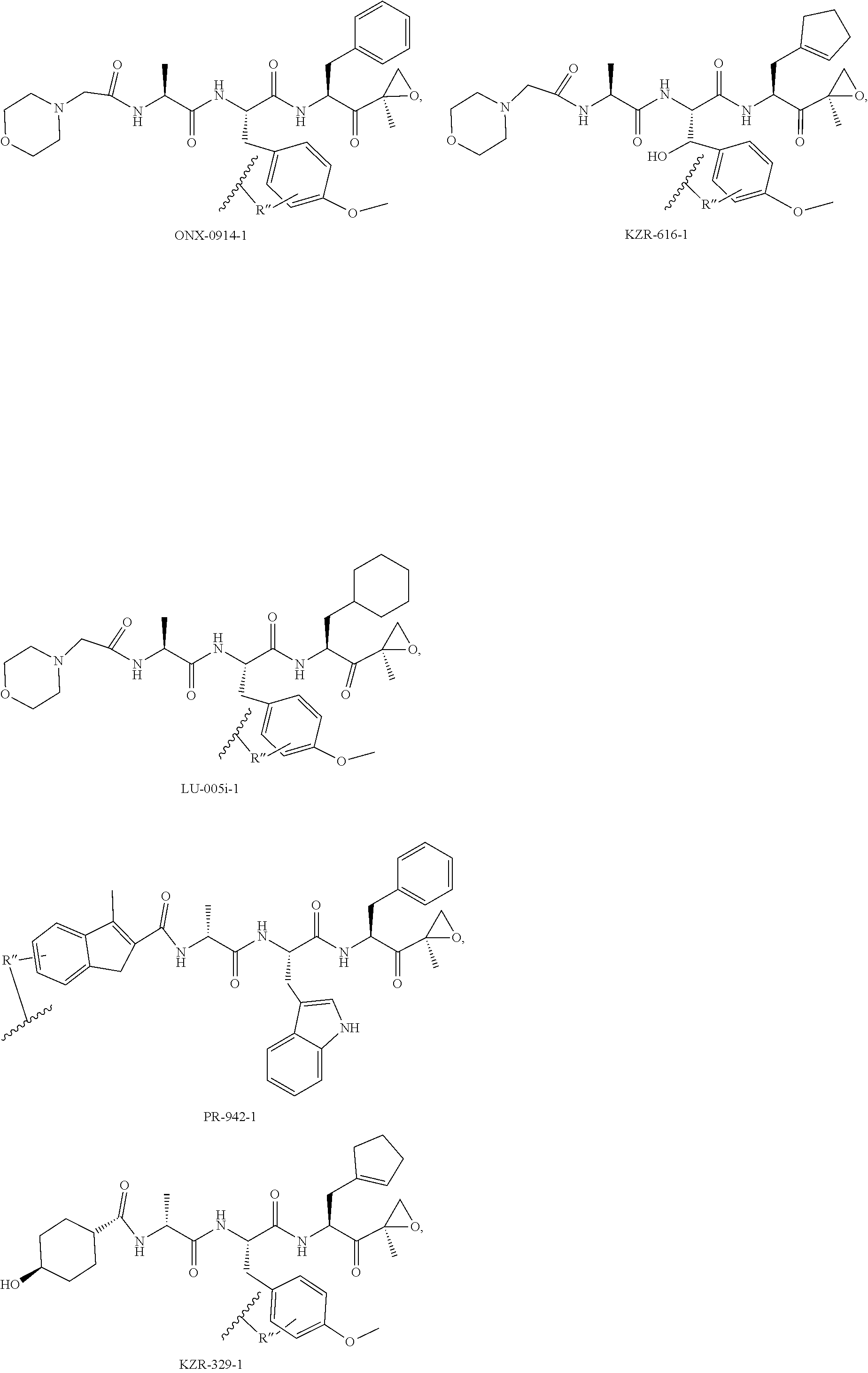 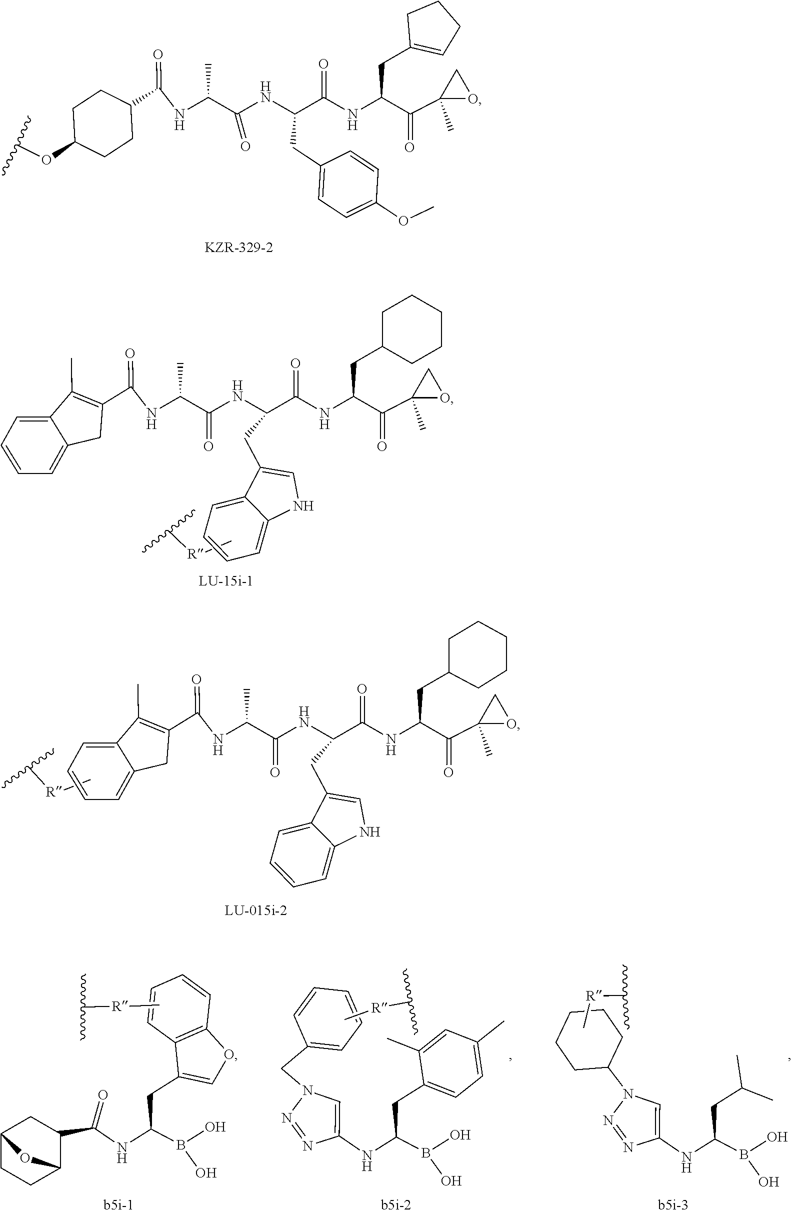 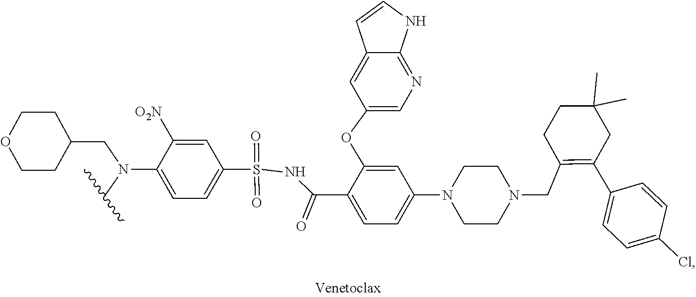 [SMILES: *N(CC1CCOCC1)c1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1[N+](=O)[O-]]
or hydrated salts; or the polymorphic crystalline structures of these compounds; or the optical isomers, racemates, diastereomers or enantiomers; [symbol] is a linkage site that links to L₁ and/or L₂ independently.

32. The antibody drug conjugate according to claim 1, wherein the cytotoxic drug, D₁ and D₂, are independently selected from, A Toll-like receptor (TLR) Agonist/Antagonist and/or STING agonist, having the following formula: 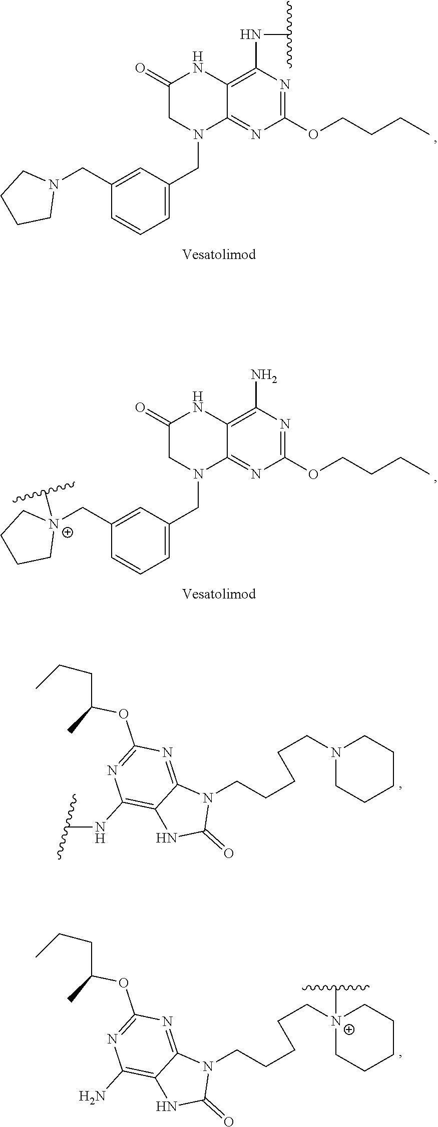 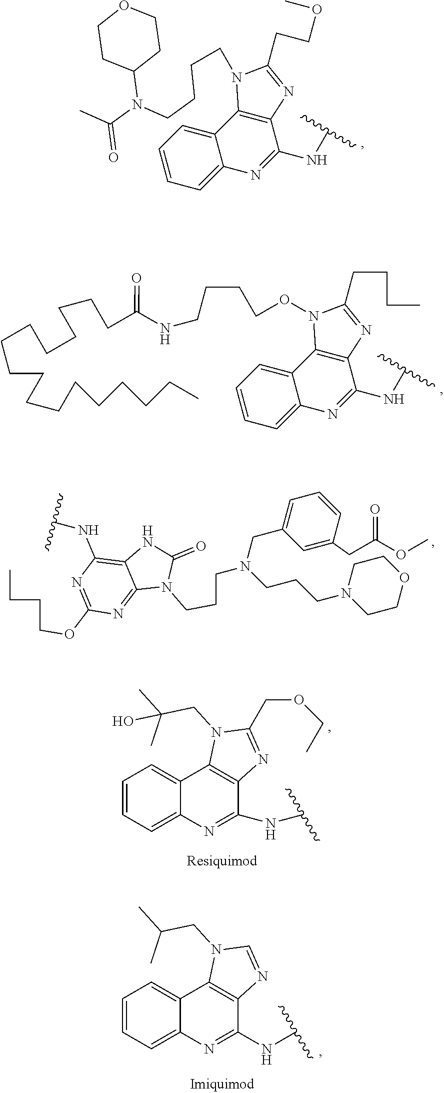 [SMILES: *Nc1nc2ccccc2c2c1nc(CCOC)n2CCCCN(C(C)=O)C1CCOCC1 ; *Nc1nc2ccccc2c2c1nc(CCCC)n2OCCCCNC(=O)CCCCCCCCCCCCCCCCC ; *Nc1nc(OCCCC)nc2c1[nH]c(=O)n2CCCN(CCCN1CCOCC1)Cc1cccc(CC(=O)OC)c1 ; *Nc1nc2ccccc2c2c1nc(COCC)n2CC(C)(C)O ; *Nc1nc2ccccc2c2c1ncn2CC(C)C] 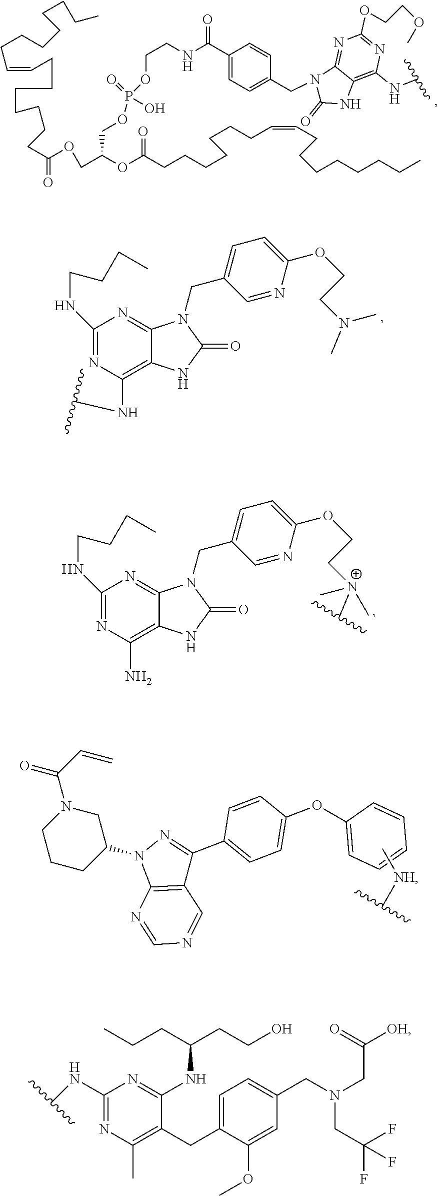 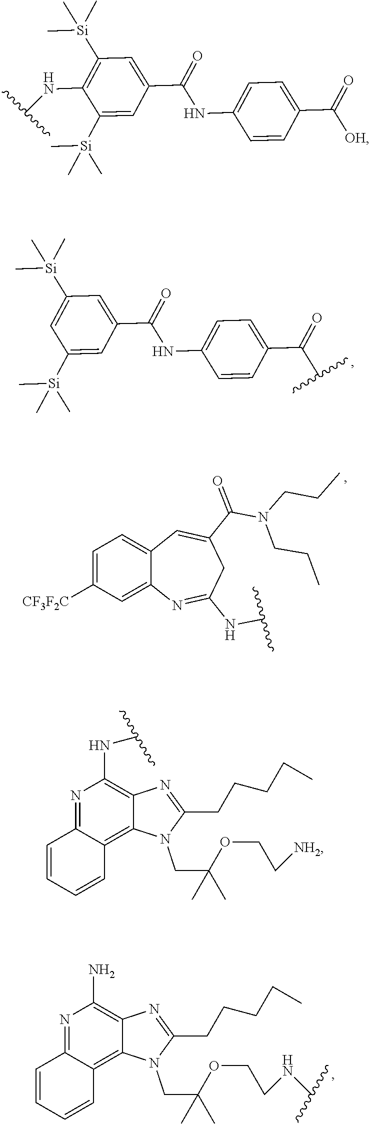 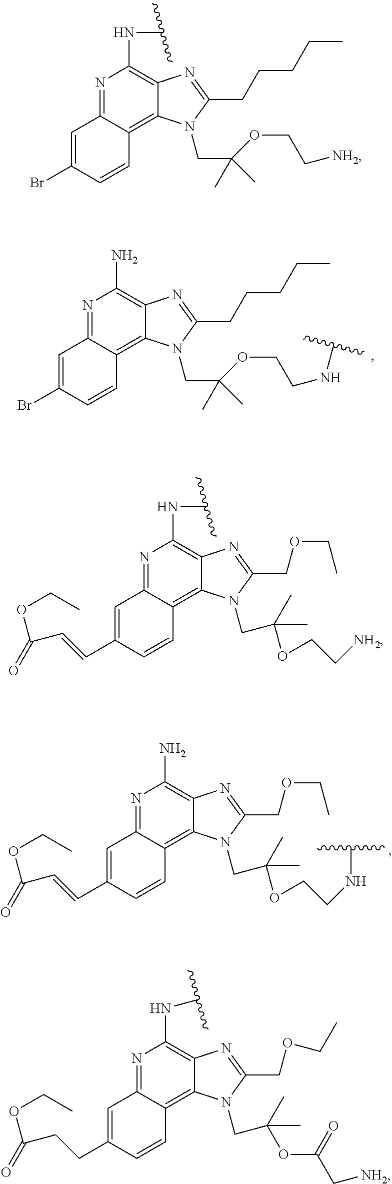 [SMILES: *Nc1nc2cc(Br)ccc2c2c1nc(CCCCC)n2CC(C)(C)OCCN ; *NCCOC(C)(C)Cn1c(CCCCC)nc2c(N)nc3cc(Br)ccc3c21 ; *Nc1nc2cc(/C=C/C(=O)OCC)ccc2c2c1nc(COCC)n2CC(C)(C)OCCN ; *NCCOC(C)(C)Cn1c(COCC)nc2c(N)nc3cc(/C=C/C(=O)OCC)ccc3c21 ; *Nc1nc2cc(CCC(=O)OCC)ccc2c2c1nc(COCC)n2CC(C)(C)OC(=O)CN] 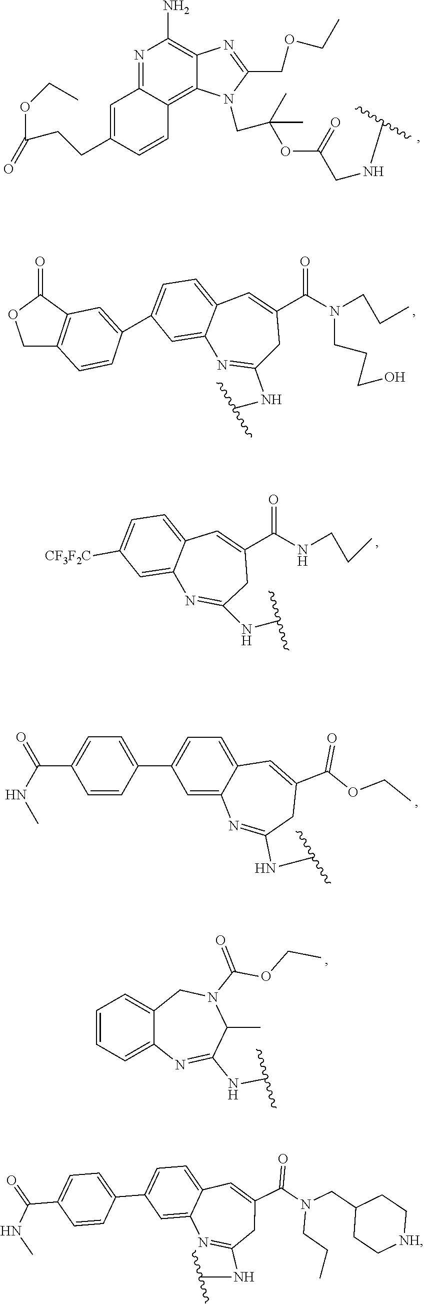 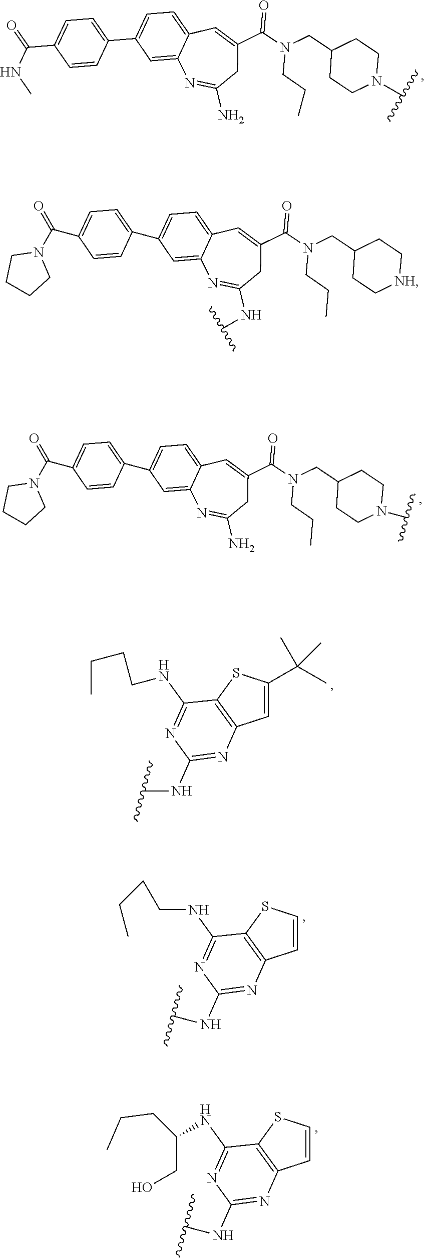 [SMILES: *N1CCC(CN(CCC)C(=O)C2=Cc3ccc(-c4ccc(C(=O)NC)cc4)cc3N=C(N)C2)CC1 ; *NC1=Nc2cc(-c3ccc(C(=O)N4CCCC4)cc3)ccc2C=C(C(=O)N(CCC)CC2CCNCC2)C1 ; *N1CCC(CN(CCC)C(=O)C2=Cc3ccc(-c4ccc(C(=O)N5CCCC5)cc4)cc3N=C(N)C2)CC1 ; *Nc1nc(NCCCC)c2sc(C(C)(C)C)cc2n1 ; *Nc1nc(NCCCC)c2sccc2n1 ; *Nc1nc(N[C@H](CO)CCC)c2sccc2n1] 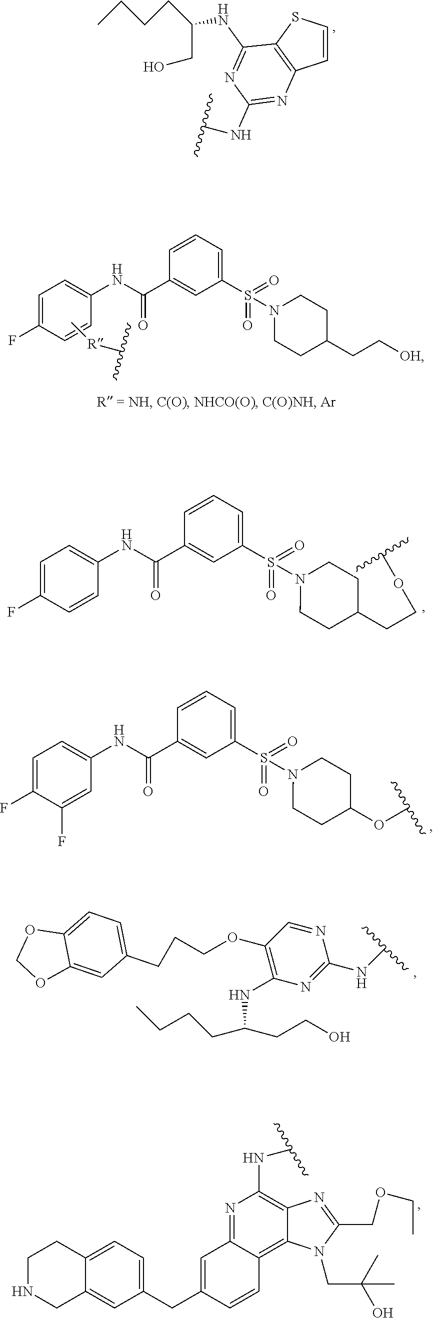 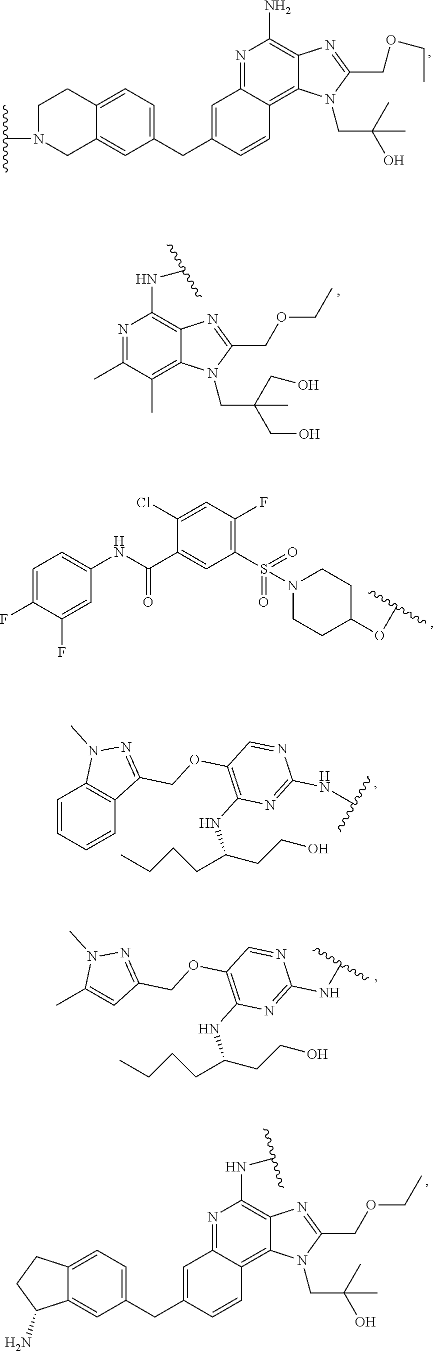 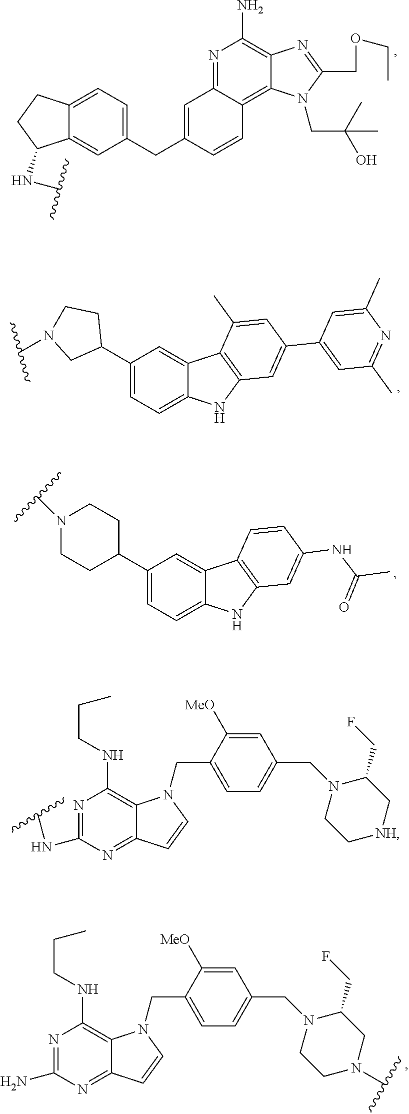 [SMILES: *N[C@@H]1CCc2ccc(Cc3ccc4c(c3)nc(N)c3nc(COCC)n(CC(C)(C)O)c34)cc21 ; *N1CCC(c2ccc3[nH]c4cc(-c5cc(C)nc(C)c5)cc(C)c4c3c2)C1 ; *N1CCC(c2ccc3[nH]c4cc(NC(C)=O)ccc4c3c2)CC1 ; *Nc1nc(NCCC)c2c(ccn2Cc2ccc(CN3CCNC[C@H]3CF)cc2OC)n1 ; *N1CCN(Cc2ccc(Cn3ccc4nc(N)nc(NCCC)c43)c(OC)c2)[C@H](CF)C1] 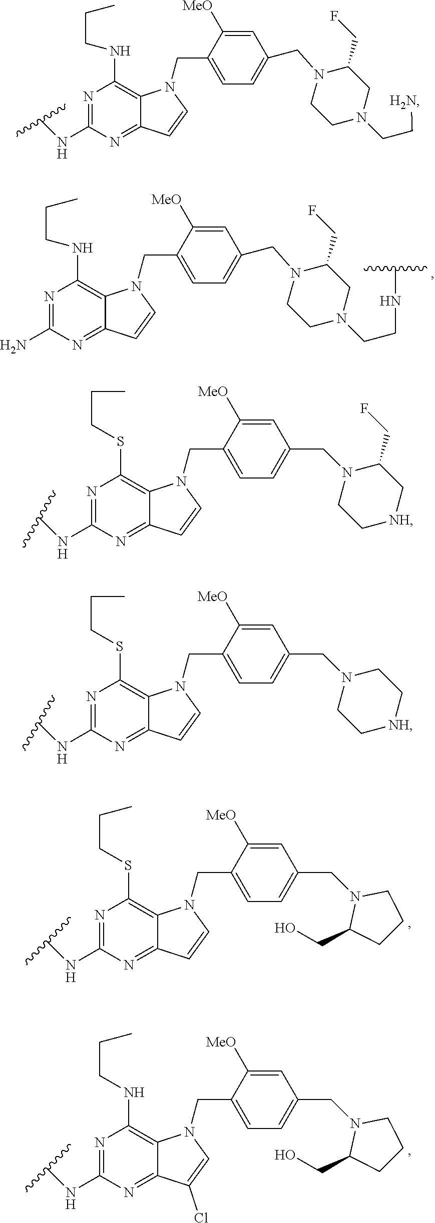 [SMILES: *Nc1nc(NCCC)c2c(ccn2Cc2ccc(CN3CCN(CCN)C[C@H]3CF)cc2OC)n1 ; *NCCN1CCN(Cc2ccc(Cn3ccc4nc(N)nc(NCCC)c43)c(OC)c2)[C@H](CF)C1 ; *Nc1nc(SCCC)c2c(ccn2Cc2ccc(CN3CCNC[C@H]3CF)cc2OC)n1 ; *Nc1nc(SCCC)c2c(ccn2Cc2ccc(CN3CCNCC3)cc2OC)n1 ; *Nc1nc(SCCC)c2c(ccn2Cc2ccc(CN3CCC[C@H]3CO)cc2OC)n1 ; *Nc1nc(NCCC)c2c(n1)c(Cl)cn2Cc1ccc(CN2CCC[C@H]2CO)cc1OC] 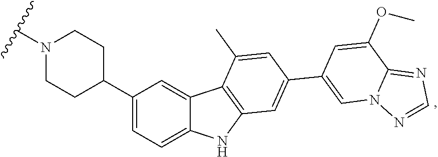 [SMILES: *N1CCC(c2ccc3[nH]c4cc(-c5cc(OC)c6ncnn6c5)cc(C)c4c3c2)CC1] 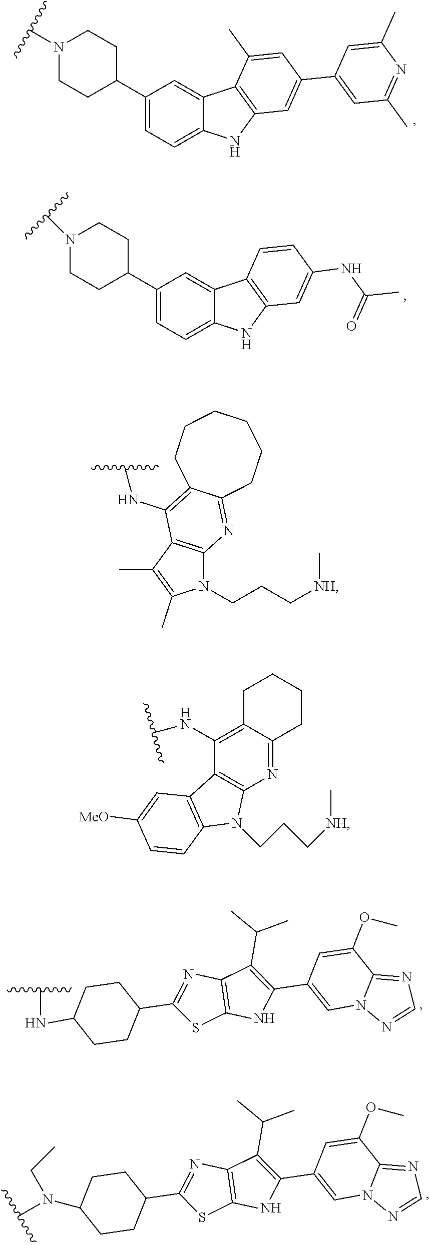 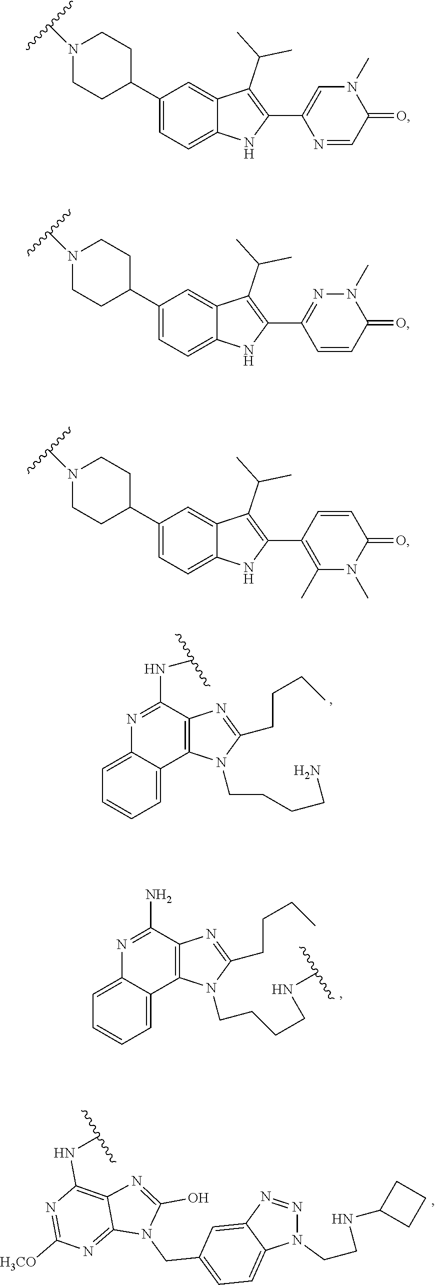 [SMILES: *N1CCC(c2ccc3[nH]c(-c4cn(C)c(=O)cn4)c(C(C)C)c3c2)CC1 ; *N1CCC(c2ccc3[nH]c(-c4ccc(=O)n(C)n4)c(C(C)C)c3c2)CC1 ; *N1CCC(c2ccc3[nH]c(-c4ccc(=O)n(C)c4C)c(C(C)C)c3c2)CC1 ; *Nc1nc2ccccc2c2c1nc(CCCC)n2CCCCN ; *NCCCCn1c(CCCC)nc2c(N)nc3ccccc3c21 ; *Nc1nc(OC)nc2c1nc(O)n2Cc1ccc2c(c1)nnn2CCNC1CCC1] 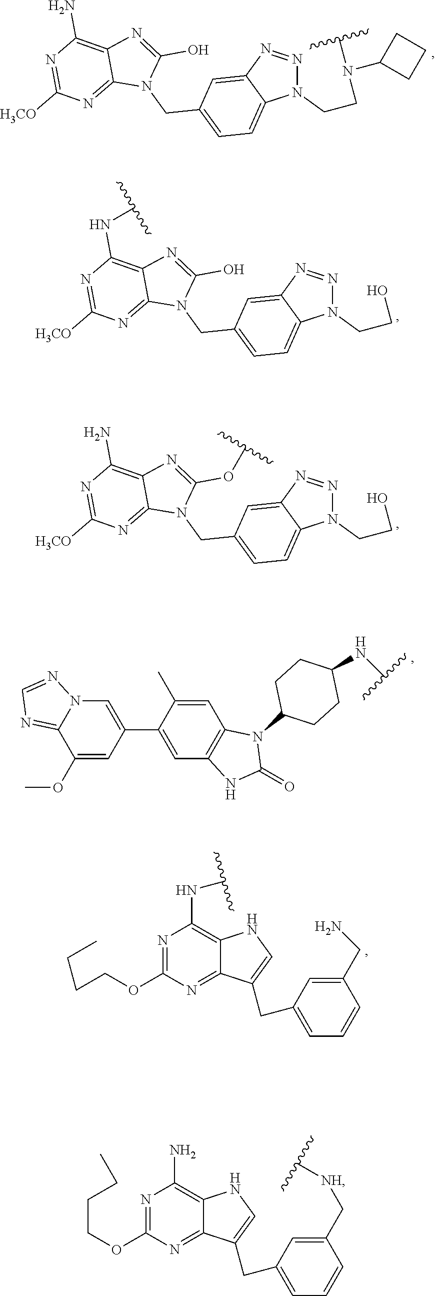 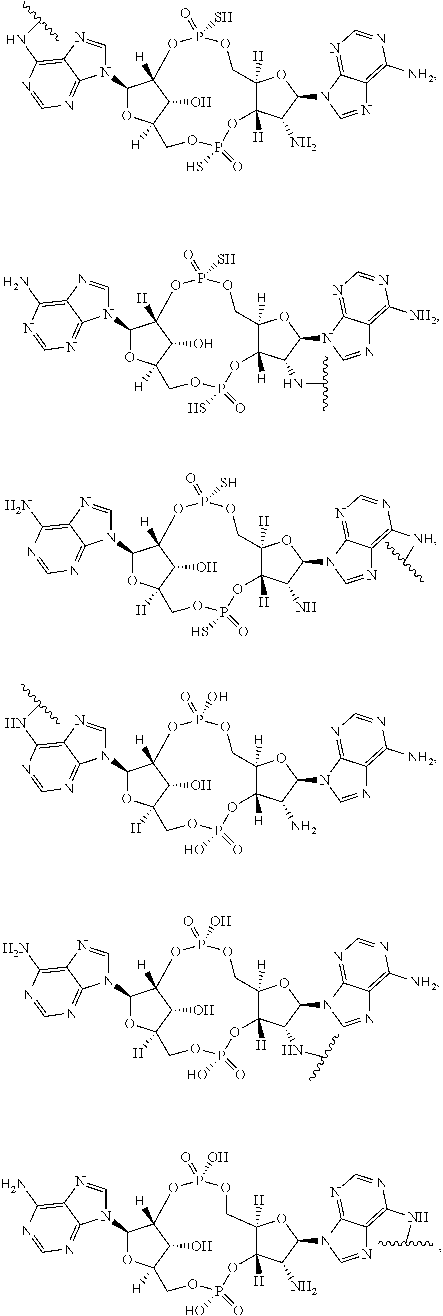 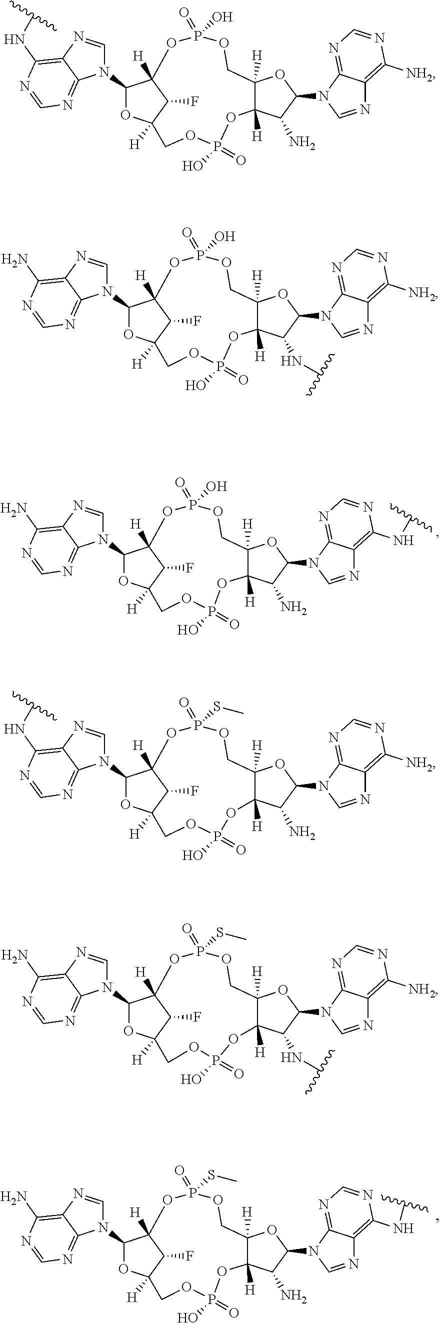 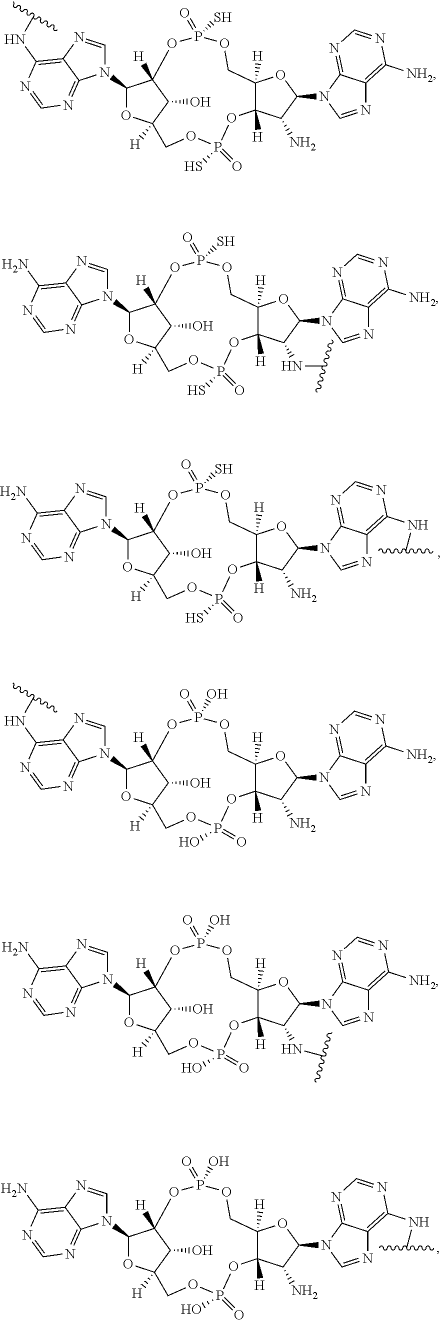 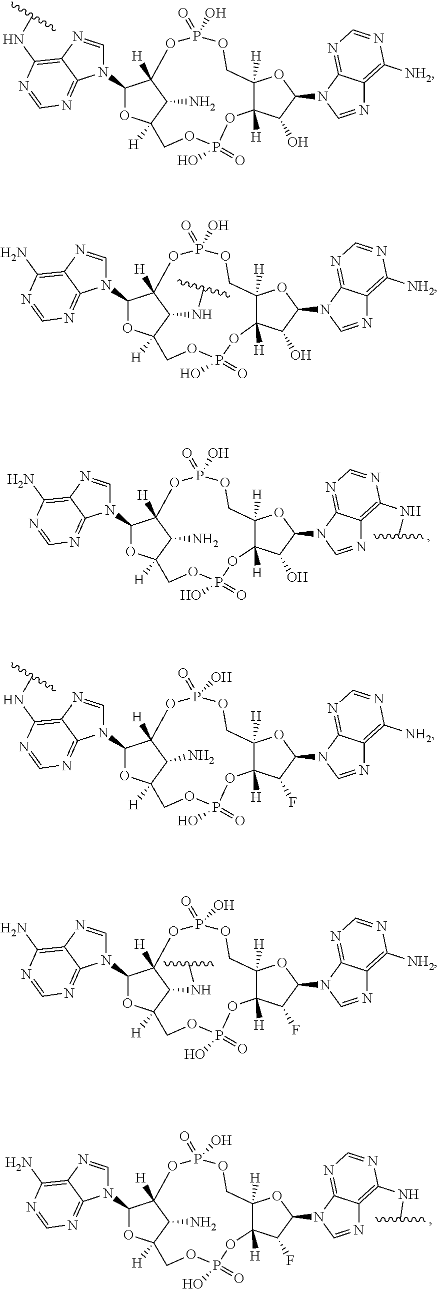 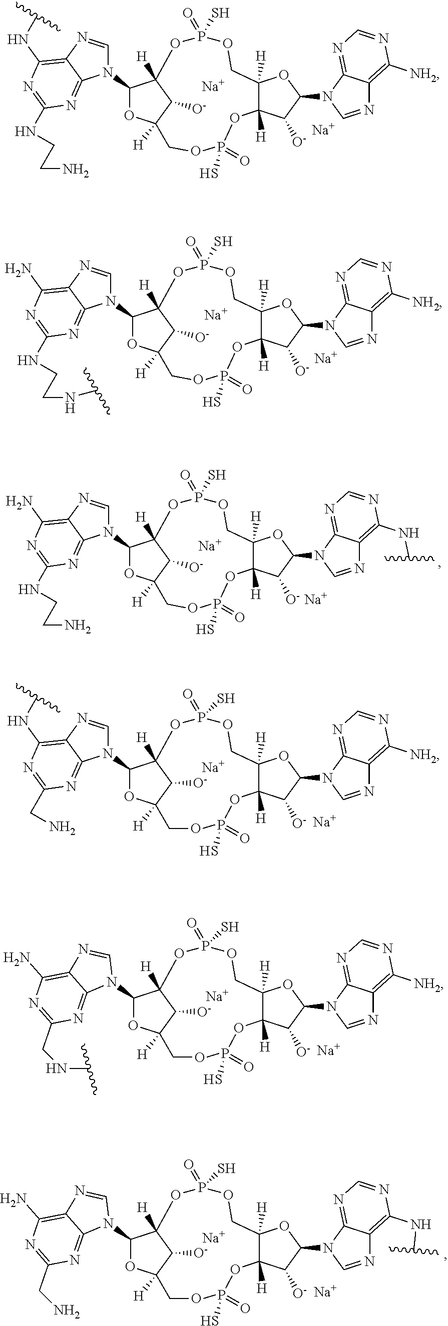 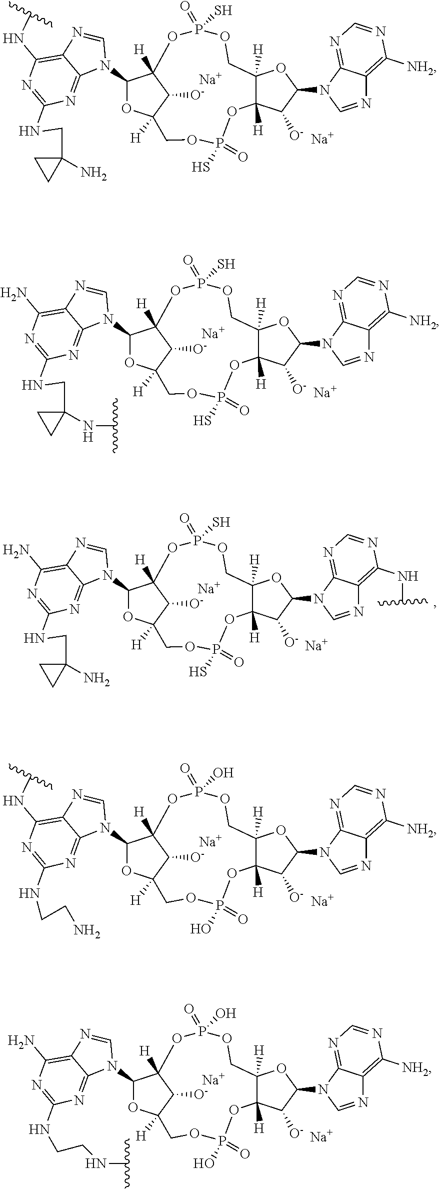 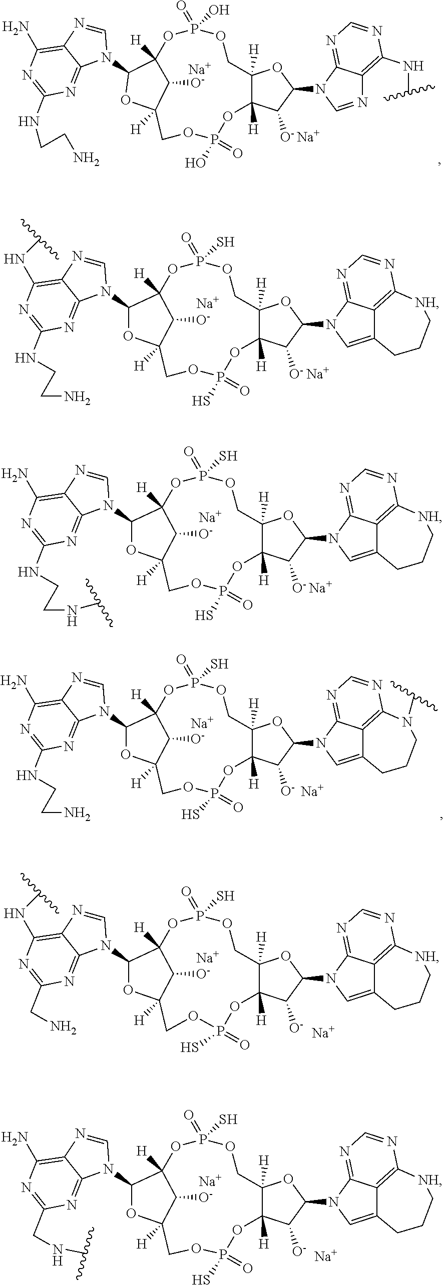 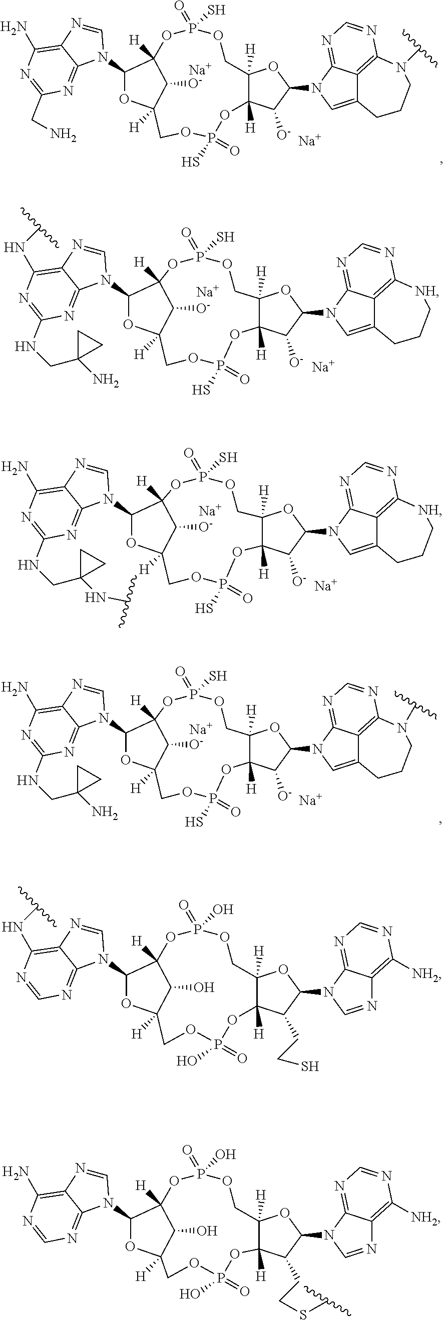 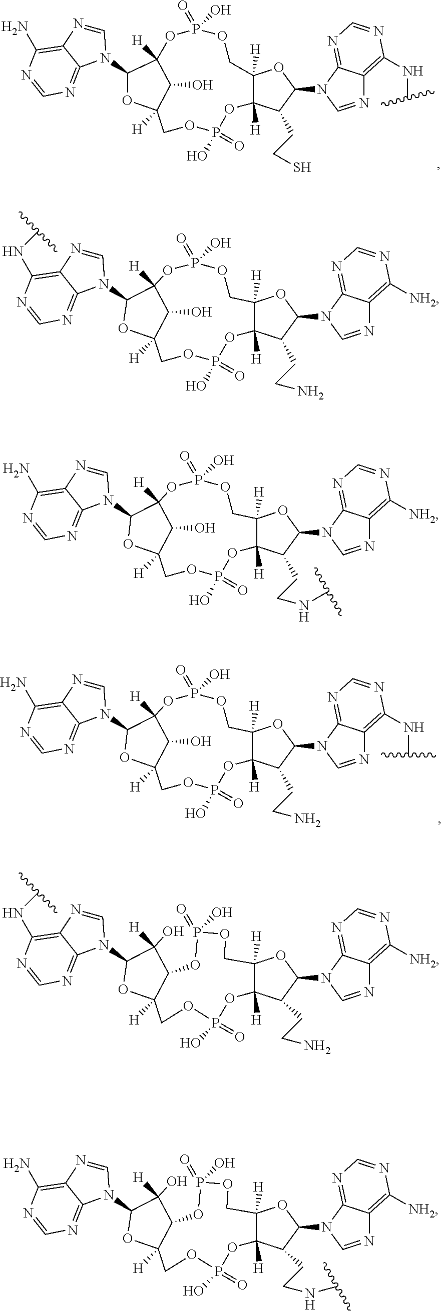 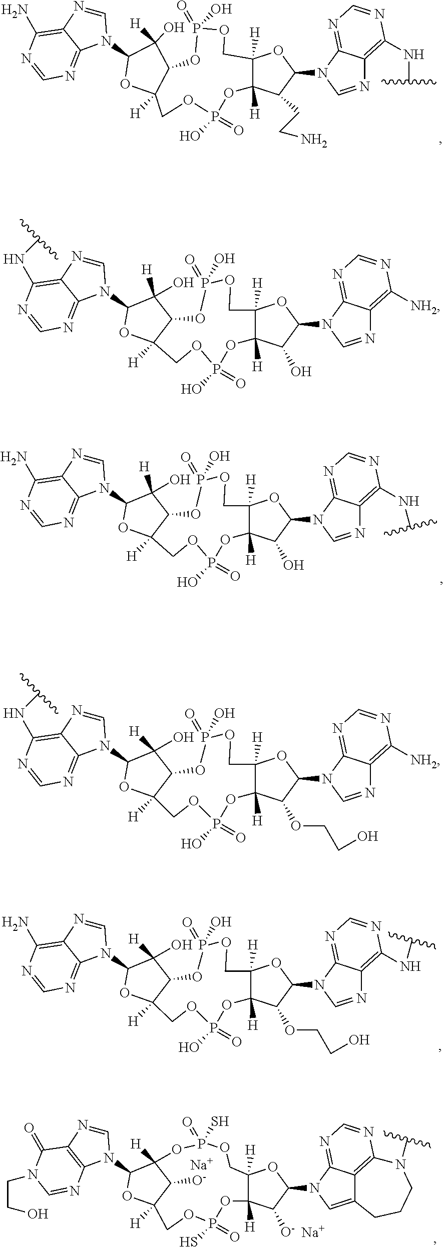 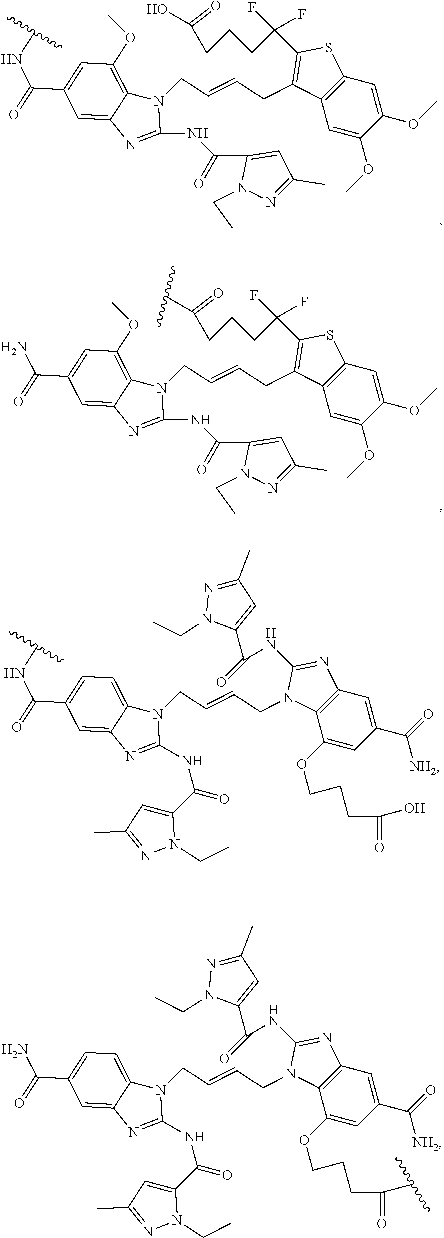 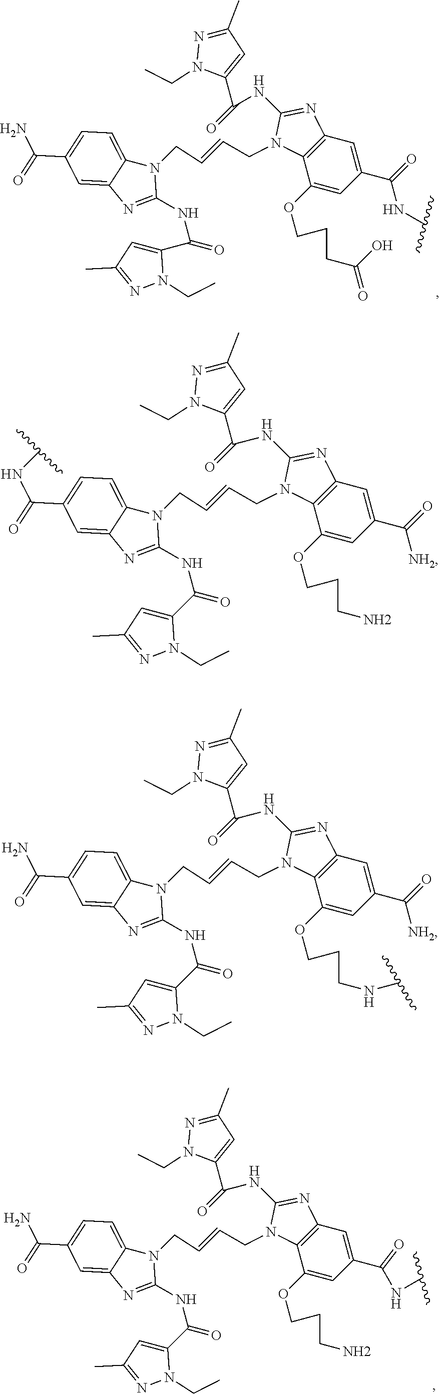 [SMILES: *NC(=O)c1cc(OCCCC(=O)O)c2c(c1)nc(NC(=O)c1cc(C)nn1CC)n2C/C=C/Cn1c(NC(=O)c2cc(C)nn2CC)nc2cc(C(N)=O)ccc21 ; *NC(=O)c1ccc2c(c1)nc(NC(=O)c1cc(C)nn1CC)n2C/C=C/Cn1c(NC(=O)c2cc(C)nn2CC)nc2cc(C(N)=O)cc(OCCCN)c21 ; *NCCCOc1cc(C(N)=O)cc2nc(NC(=O)c3cc(C)nn3CC)n(C/C=C/Cn3c(NC(=O)c4cc(C)nn4CC)nc4cc(C(N)=O)ccc43)c12 ; *NC(=O)c1cc(OCCCN)c2c(c1)nc(NC(=O)c1cc(C)nn1CC)n2C/C=C/Cn1c(NC(=O)c2cc(C)nn2CC)nc2cc(C(N)=O)ccc21] 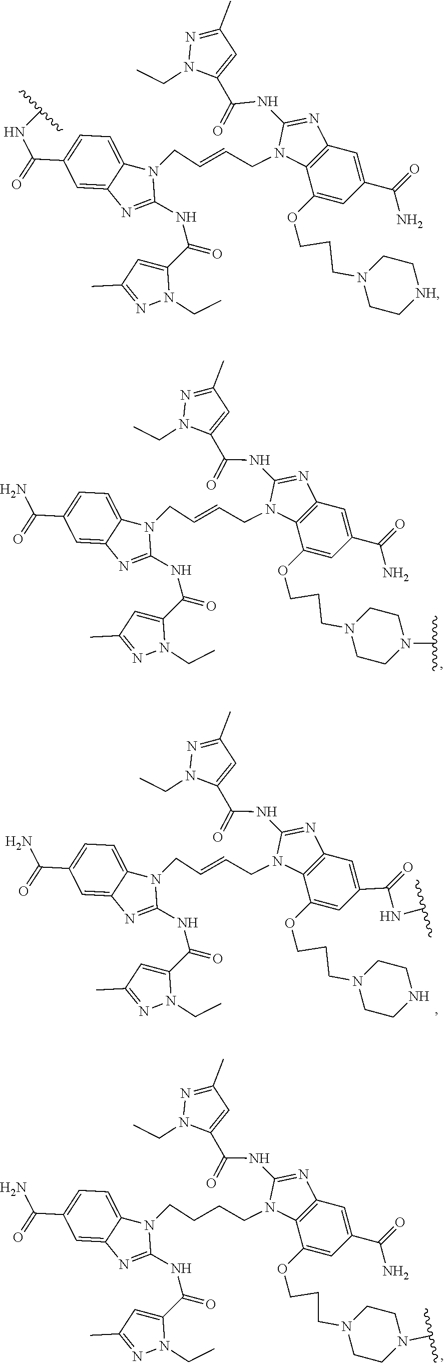 [SMILES: *NC(=O)c1ccc2c(c1)nc(NC(=O)c1cc(C)nn1CC)n2C/C=C/Cn1c(NC(=O)c2cc(C)nn2CC)nc2cc(C(N)=O)cc(OCCCN3CCNCC3)c21 ; *N1CCN(CCCOc2cc(C(N)=O)cc3nc(NC(=O)c4cc(C)nn4CC)n(C/C=C/Cn4c(NC(=O)c5cc(C)nn5CC)nc5cc(C(N)=O)ccc54)c23)CC1 ; *NC(=O)c1cc(OCCCN2CCNCC2)c2c(c1)nc(NC(=O)c1cc(C)nn1CC)n2C/C=C/Cn1c(NC(=O)c2cc(C)nn2CC)nc2cc(C(N)=O)ccc21 ; *N1CCN(CCCOc2cc(C(N)=O)cc3nc(NC(=O)c4cc(C)nn4CC)n(CCCCn4c(NC(=O)c5cc(C)nn5CC)nc5cc(C(N)=O)ccc54)c23)CC1] 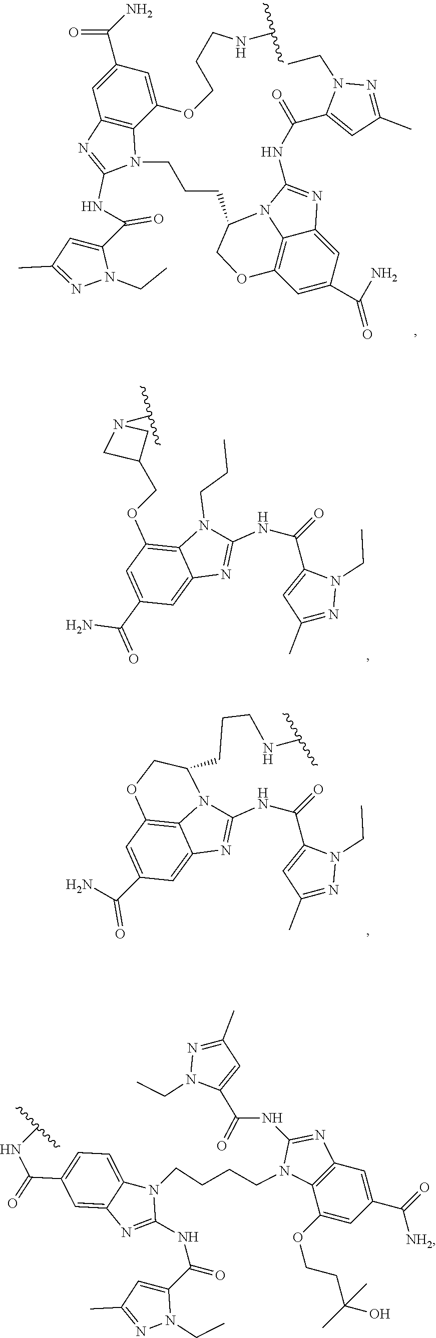 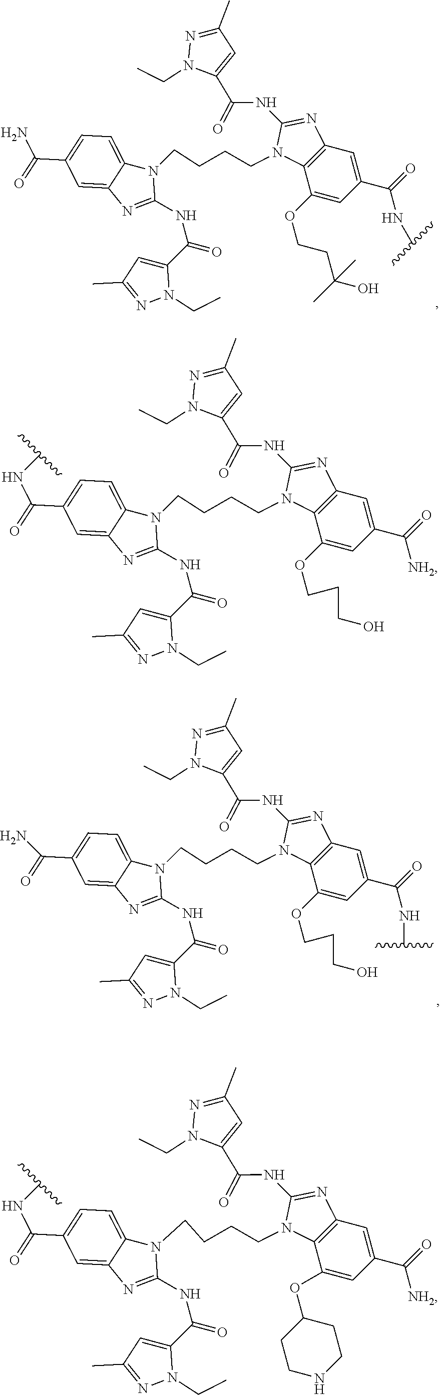 [SMILES: *NC(=O)c1cc(OCCC(C)(C)O)c2c(c1)nc(NC(=O)c1cc(C)nn1CC)n2CCCCn1c(NC(=O)c2cc(C)nn2CC)nc2cc(C(N)=O)ccc21 ; *NC(=O)c1ccc2c(c1)nc(NC(=O)c1cc(C)nn1CC)n2CCCCn1c(NC(=O)c2cc(C)nn2CC)nc2cc(C(N)=O)cc(OCCCO)c21 ; *NC(=O)c1cc(OCCCO)c2c(c1)nc(NC(=O)c1cc(C)nn1CC)n2CCCCn1c(NC(=O)c2cc(C)nn2CC)nc2cc(C(N)=O)ccc21 ; *NC(=O)c1ccc2c(c1)nc(NC(=O)c1cc(C)nn1CC)n2CCCCn1c(NC(=O)c2cc(C)nn2CC)nc2cc(C(N)=O)cc(OC3CCNCC3)c21] 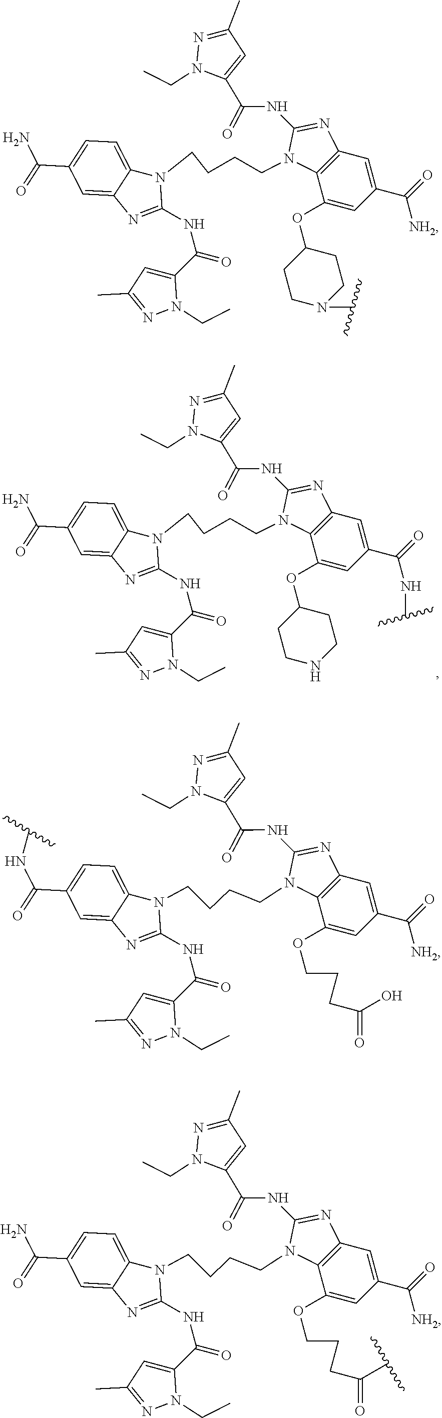 [SMILES: *N1CCC(Oc2cc(C(N)=O)cc3nc(NC(=O)c4cc(C)nn4CC)n(CCCCn4c(NC(=O)c5cc(C)nn5CC)nc5cc(C(N)=O)ccc54)c23)CC1 ; *NC(=O)c1cc(OC2CCNCC2)c2c(c1)nc(NC(=O)c1cc(C)nn1CC)n2CCCCn1c(NC(=O)c2cc(C)nn2CC)nc2cc(C(N)=O)ccc21 ; *NC(=O)c1ccc2c(c1)nc(NC(=O)c1cc(C)nn1CC)n2CCCCn1c(NC(=O)c2cc(C)nn2CC)nc2cc(C(N)=O)cc(OCCCC(=O)O)c21 ; *C(=O)CCCOc1cc(C(N)=O)cc2nc(NC(=O)c3cc(C)nn3CC)n(CCCCn3c(NC(=O)c4cc(C)nn4CC)nc4cc(C(N)=O)ccc43)c12] 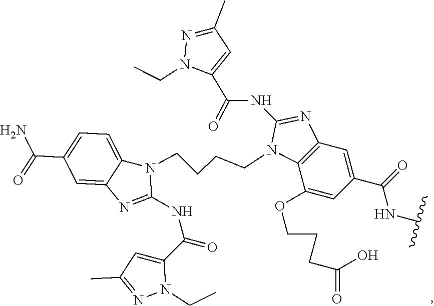 [SMILES: *NC(=O)c1cc(OCCCC(=O)O)c2c(c1)nc(NC(=O)c1cc(C)nn1CC)n2CCCCn1c(NC(=O)c2cc(C)nn2CC)nc2cc(C(N)=O)ccc21]
or hydrated salts; or the polymorphic crystalline structures of these compounds; or the optical isomers, racemates, diastereomers or enantiomers; [symbol] is a linkage site that links to L₁ and/or L₂ independently.

33. The antibody drug conjugate according to claim 1, wherein the cytotoxic drug, D₁ and D₂, are independently selected from, A proteolysis-targeting chimera (PROTAC) molecule, having the following formula: 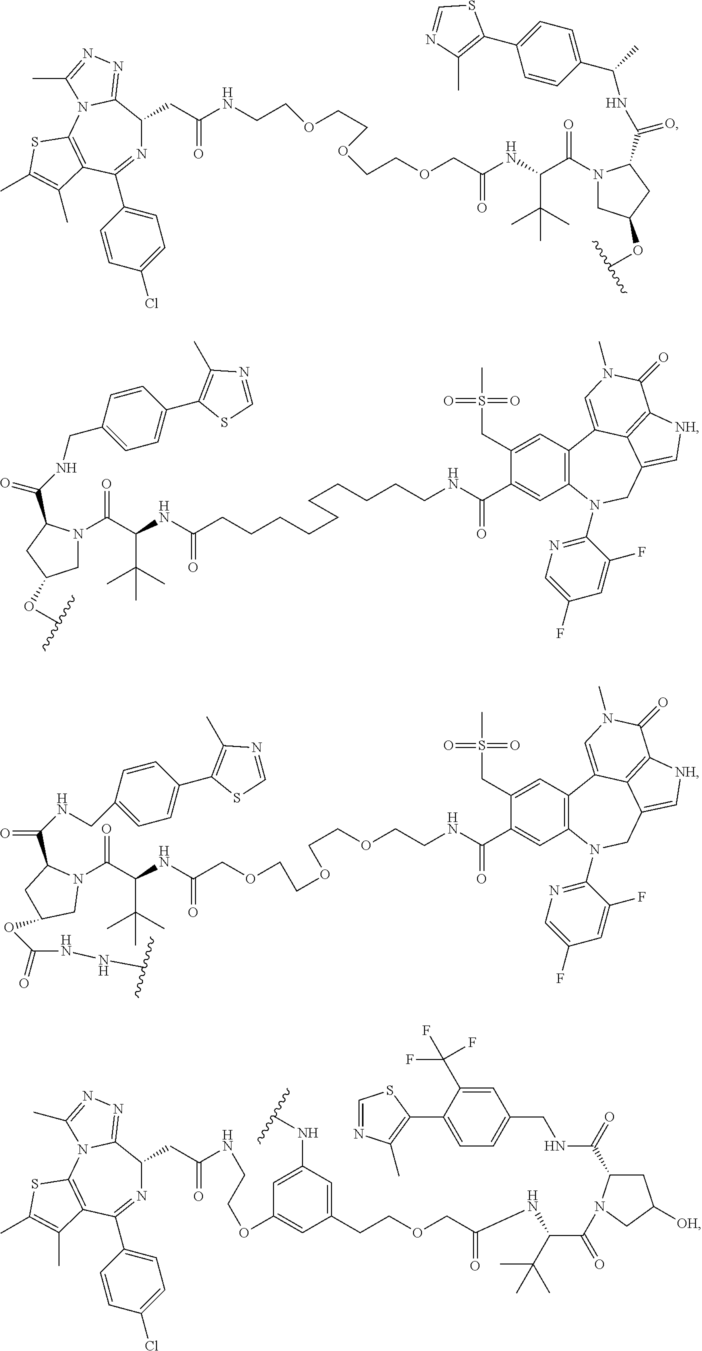 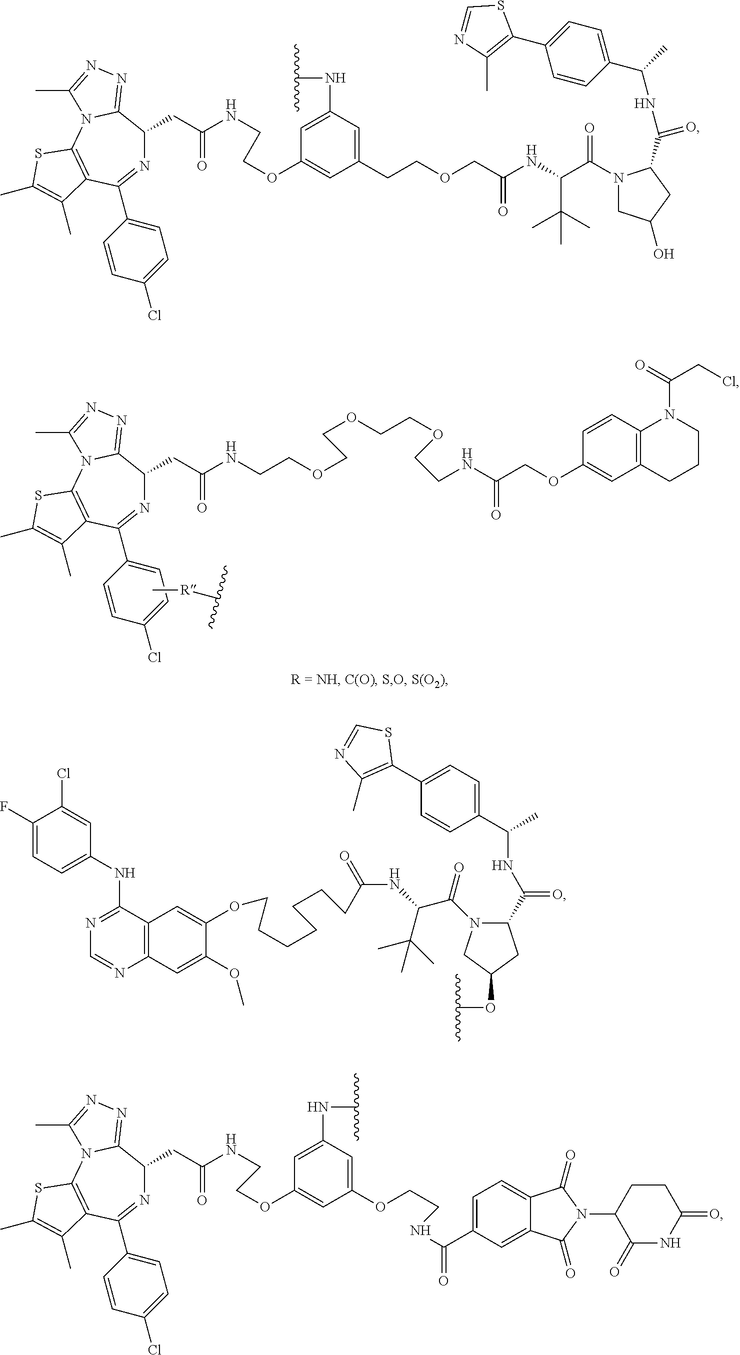 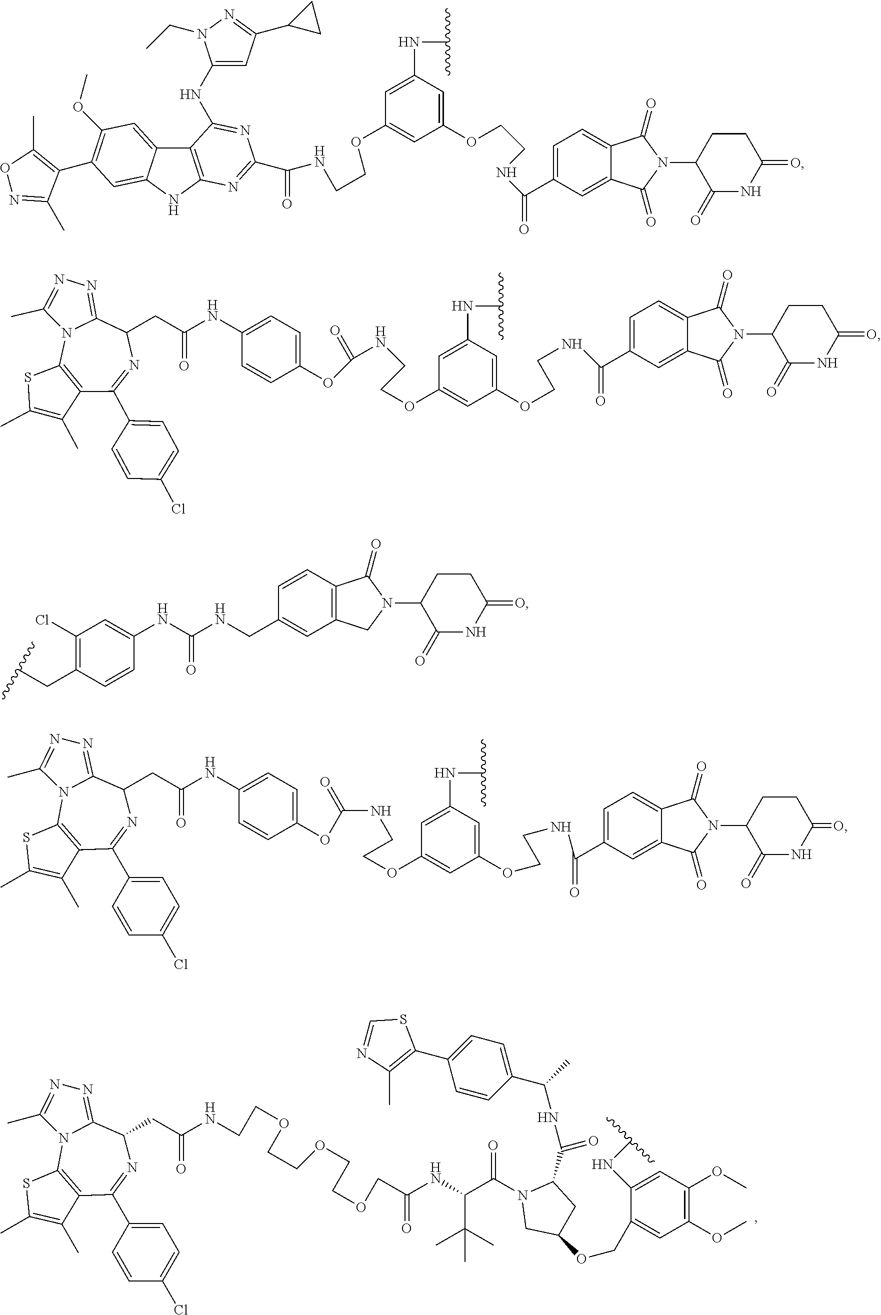 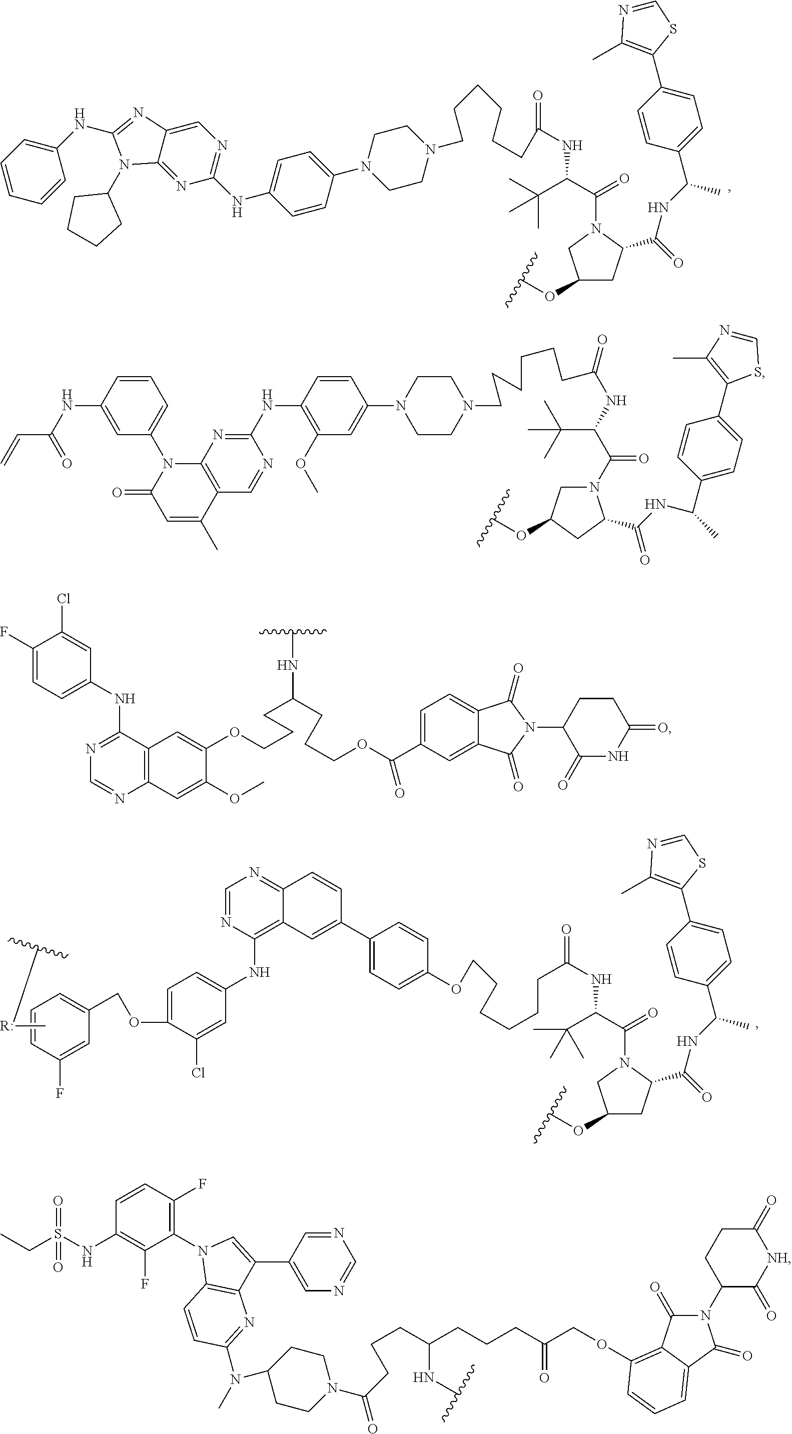 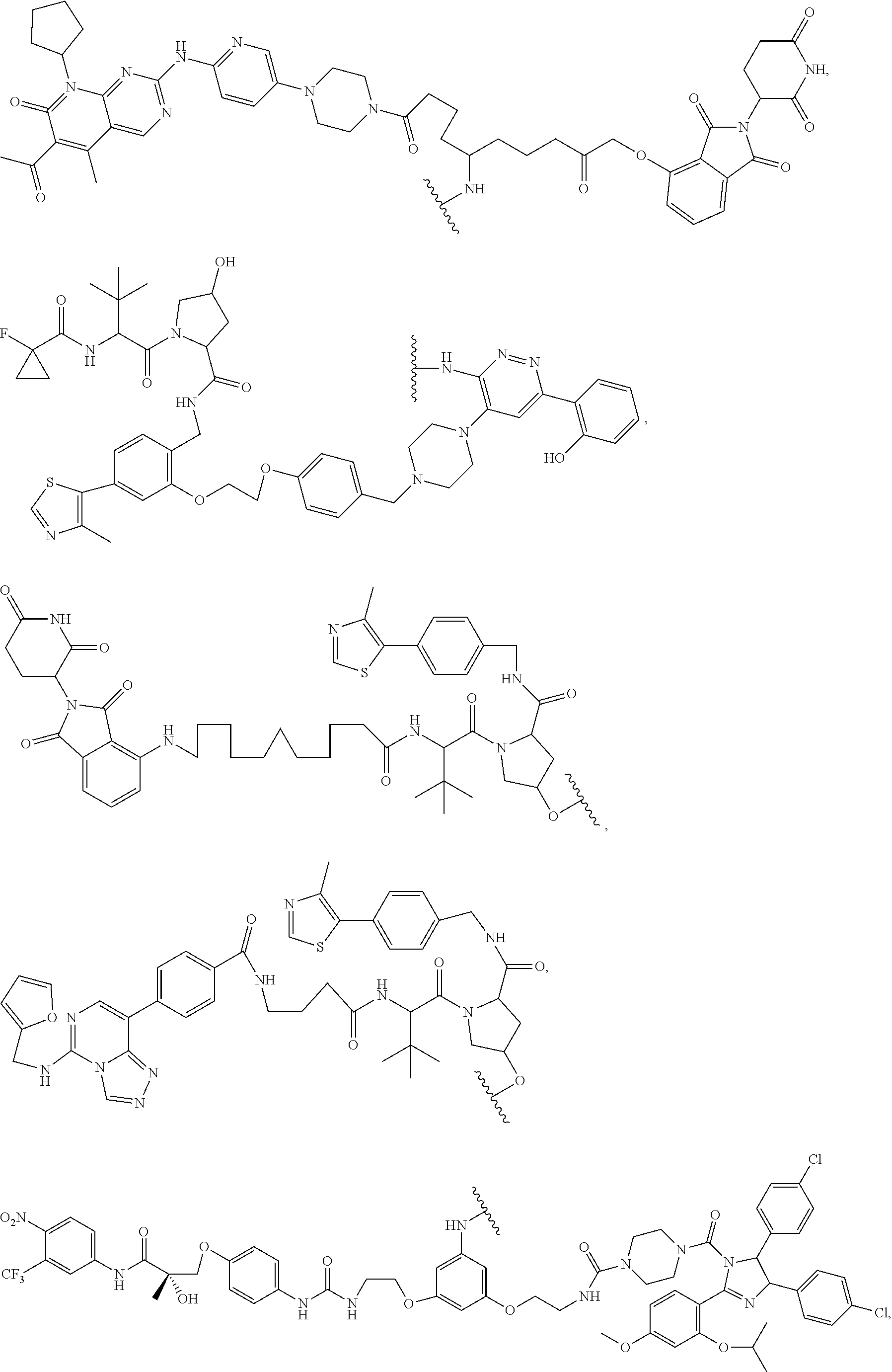 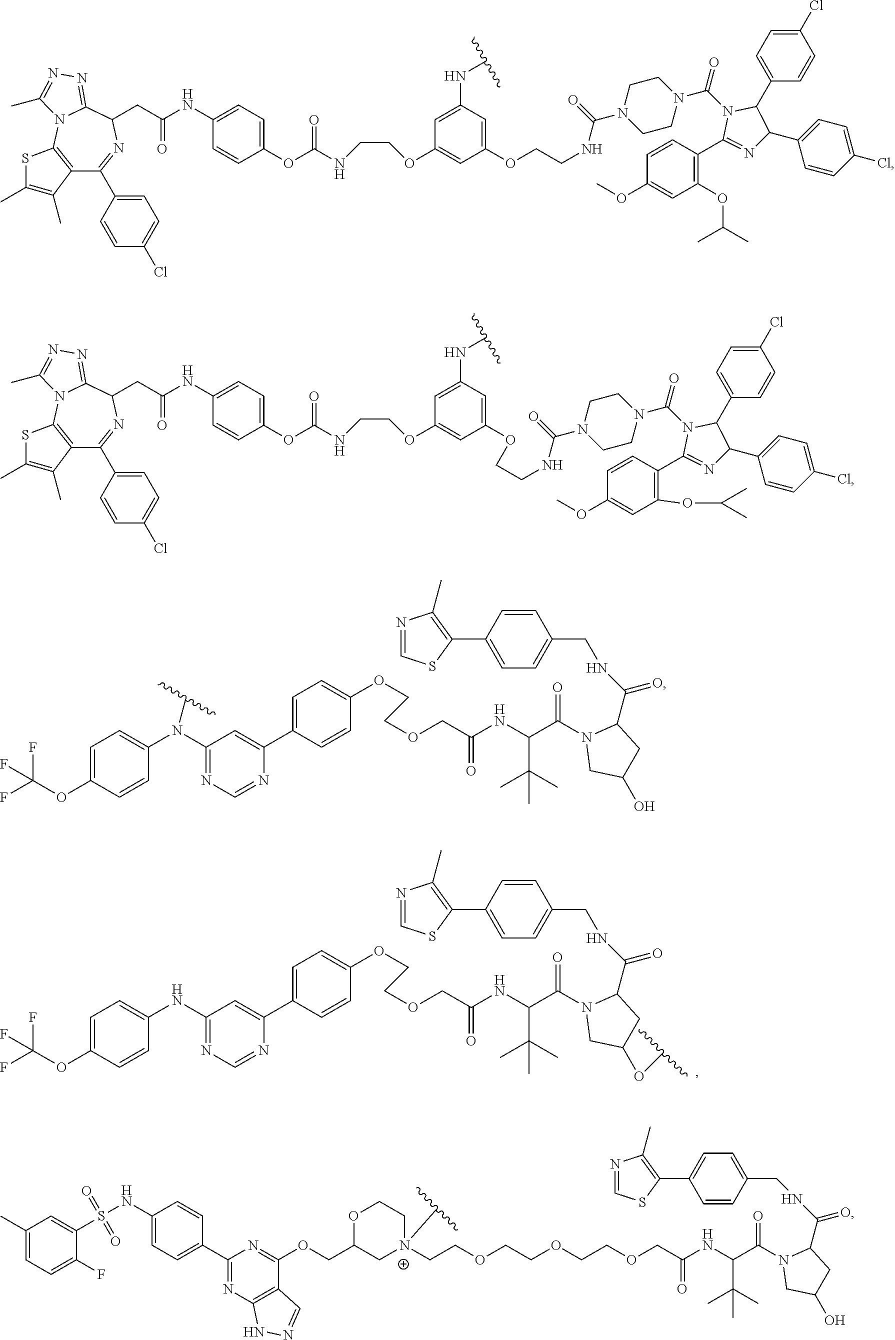 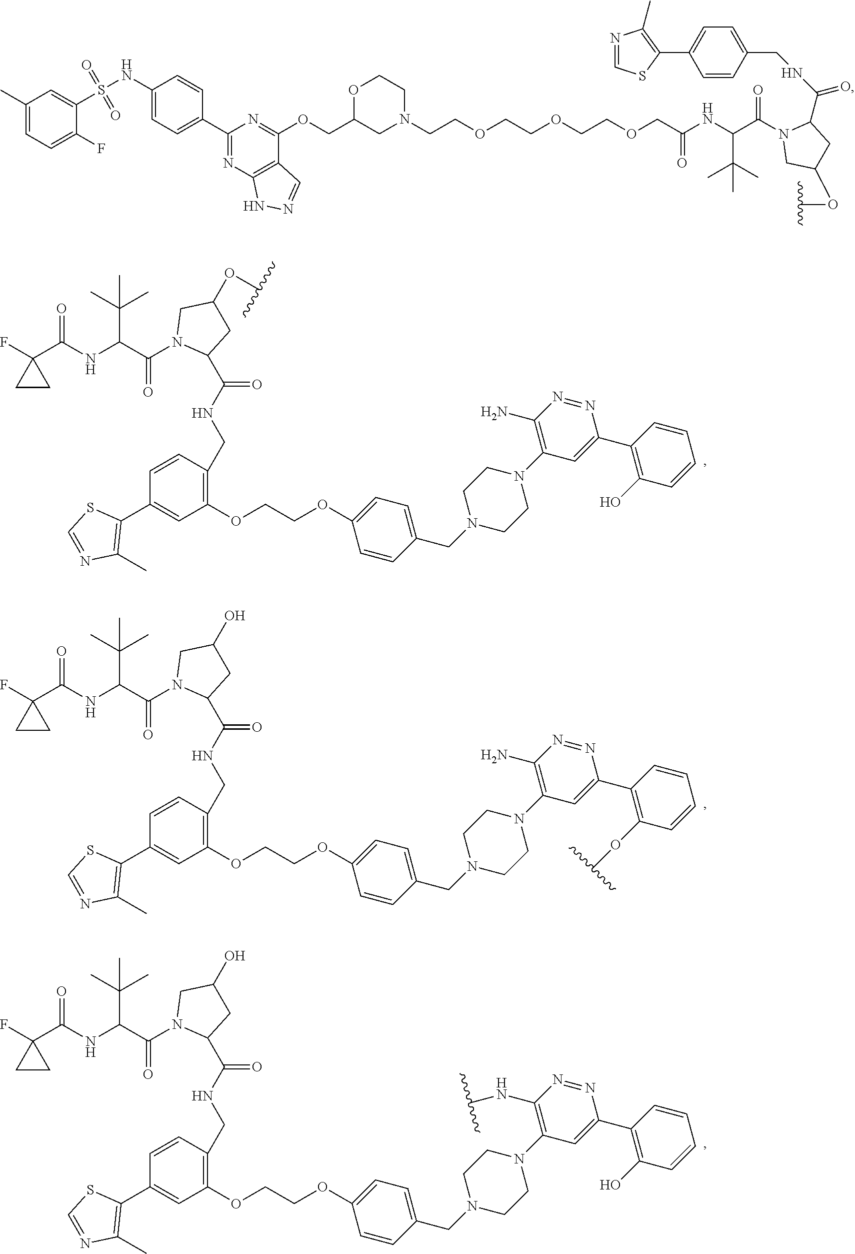 [SMILES: *OC1CC(C(=O)NCc2ccc(-c3scnc3C)cc2)N(C(=O)C(NC(=O)COCCOCCOCCN2CCOC(COc3nc(-c4ccc(NS(=O)(=O)c5cc(C)ccc5F)cc4)nc4[nH]ncc34)C2)C(C)(C)C)C1 ; *OC1CC(C(=O)NCc2ccc(-c3scnc3C)cc2OCCOc2ccc(CN3CCN(c4cc(-c5ccccc5O)nnc4N)CC3)cc2)N(C(=O)C(NC(=O)C2(F)CC2)C(C)(C)C)C1 ; *Oc1ccccc1-c1cc(N2CCN(Cc3ccc(OCCOc4cc(-c5scnc5C)ccc4CNC(=O)C4CC(O)CN4C(=O)C(NC(=O)C4(F)CC4)C(C)(C)C)cc3)CC2)c(N)nn1 ; *Nc1nnc(-c2ccccc2O)cc1N1CCN(Cc2ccc(OCCOc3cc(-c4scnc4C)ccc3CNC(=O)C3CC(O)CN3C(=O)C(NC(=O)C3(F)CC3)C(C)(C)C)cc2)CC1] 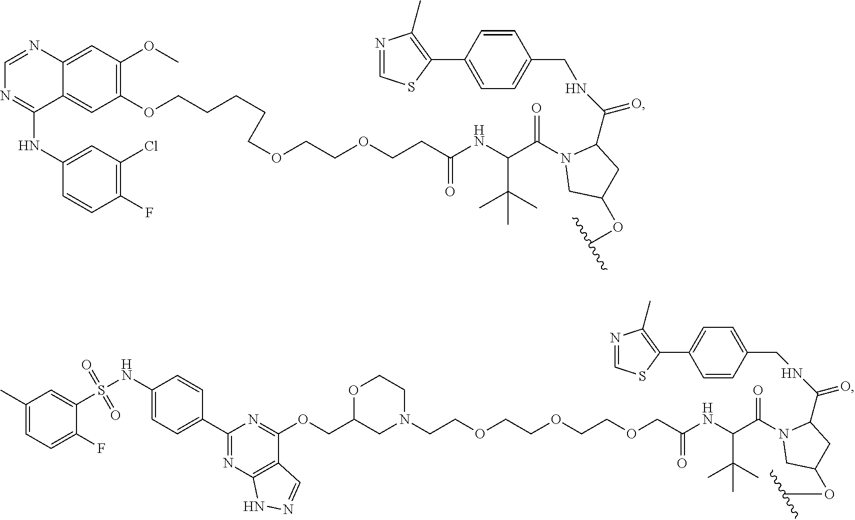 [SMILES: *OC1CC(C(=O)NCc2ccc(-c3scnc3C)cc2)N(C(=O)C(NC(=O)CCOCCOCCCCCOc2cc3c(Nc4ccc(F)c(Cl)c4)ncnc3cc2OC)C(C)(C)C)C1 ; *OC1CC(C(=O)NCc2ccc(-c3scnc3C)cc2)N(C(=O)C(NC(=O)COCCOCCOCCN2CCOC(COc3nc(-c4ccc(NS(=O)(=O)c5cc(C)ccc5F)cc4)nc4[nH]ncc34)C2)C(C)(C)C)C1]
or hydrated salts; or the polymorphic crystalline structures of these compounds; or the optical isomers, racemates, diastereomers or enantiomers; [symbol] is a linkage site that links to L₁ and/or L₂ independently.

34. The antibody drug conjugate according to claim 1, wherein the functional small molecule, A₁, A₂, A₃, A₄, A₅ and/or A₆ are independently a cytotoxic drug or a chemotherapeutic drug/molecule, when it is in different form of a structure from D₁ or D₂.

35. The antibody drug conjugate according to claim 1, wherein the functional small molecule, A₁, A₂, A₃, A₄, As and/or A₆, is independently selected from, a cell-binding molecule/ligand or a cell receptor agonist of the following molecules: 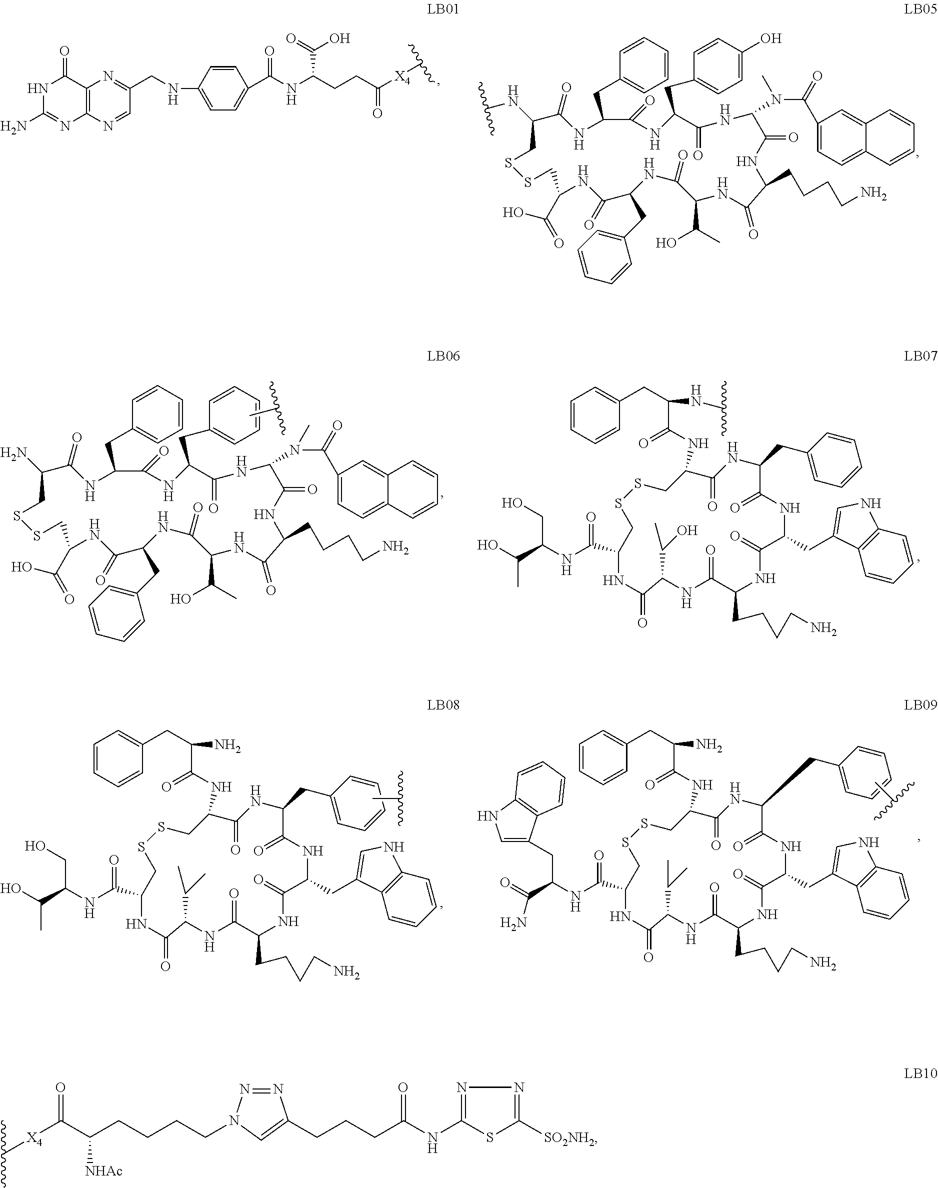 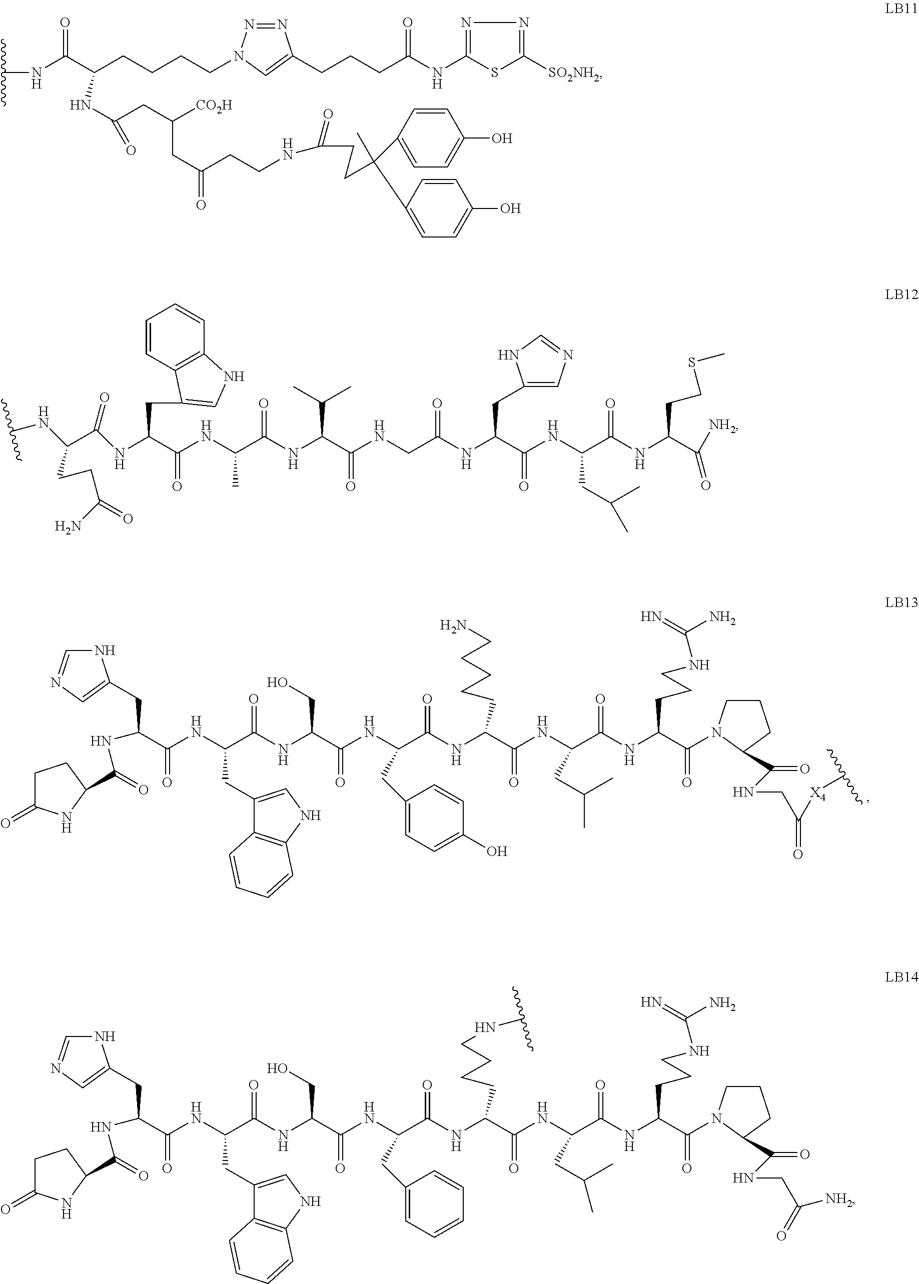 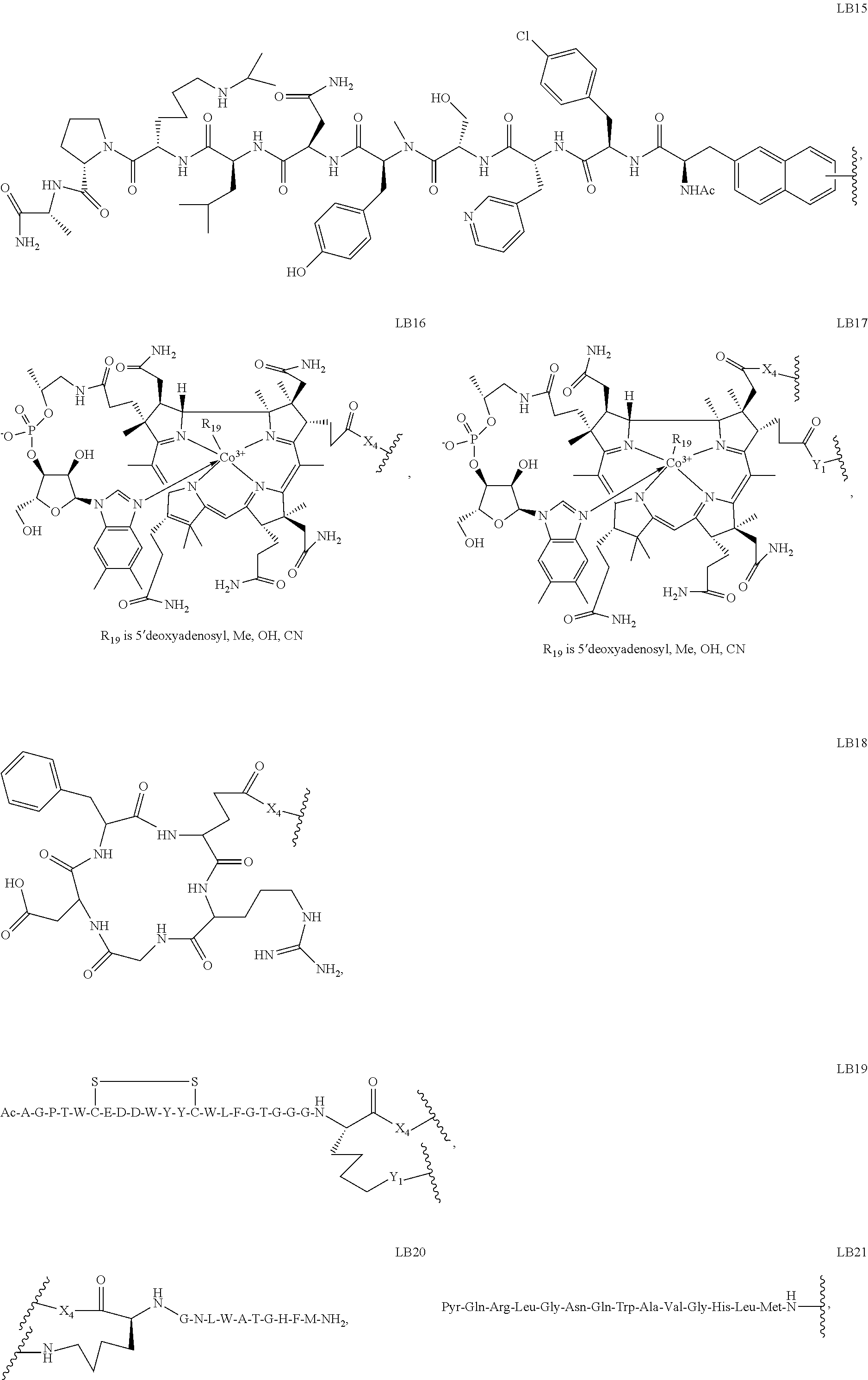 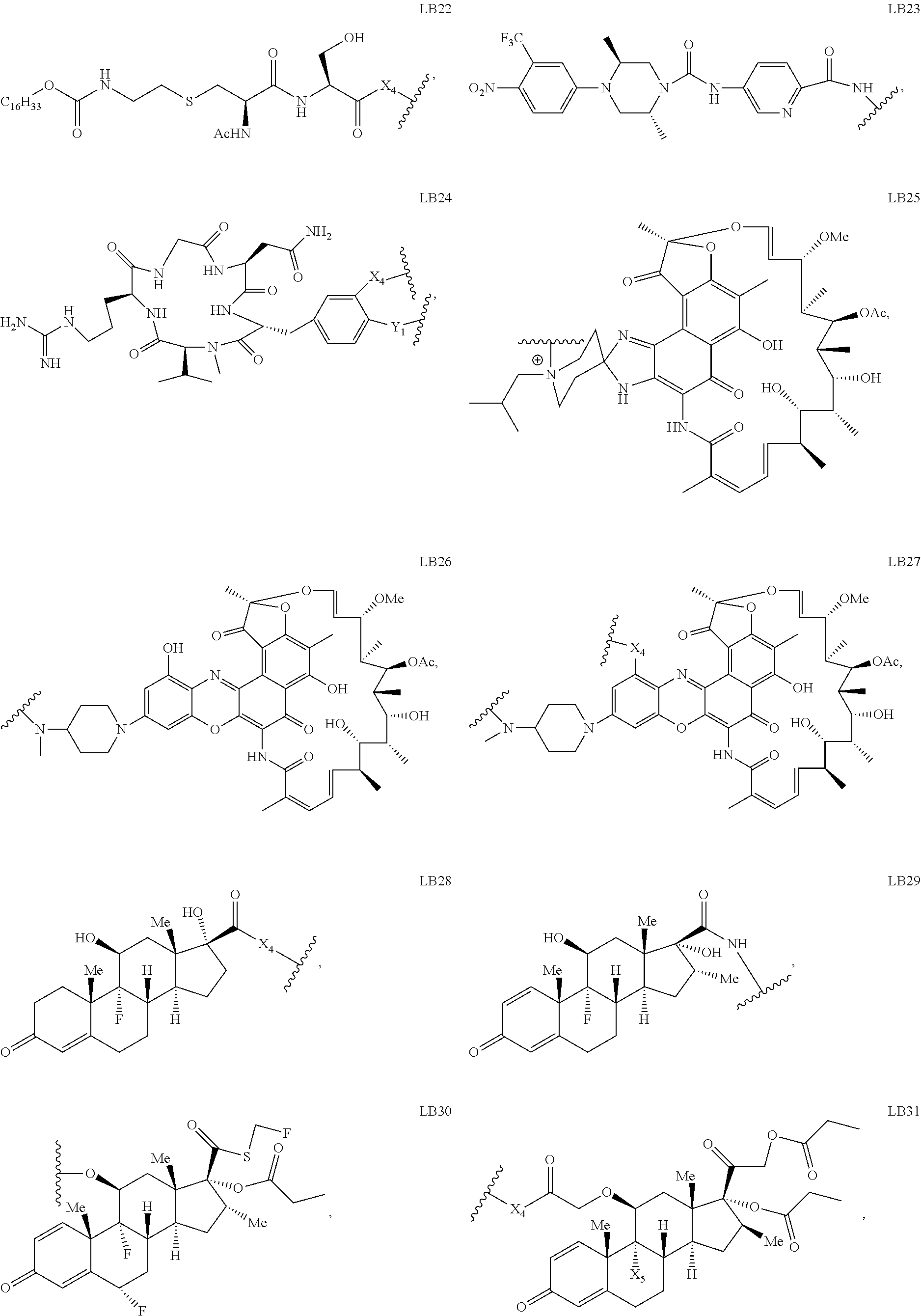 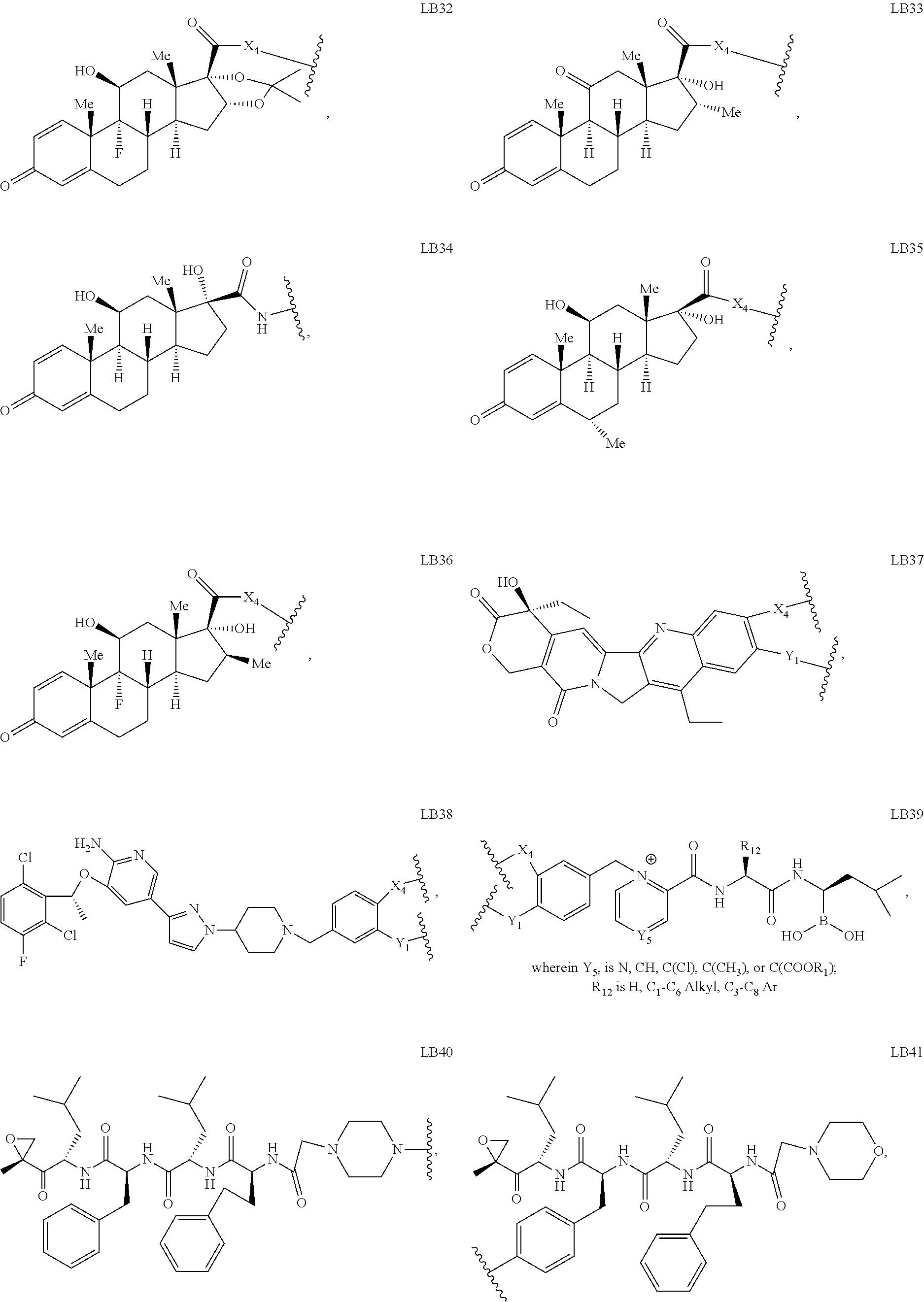 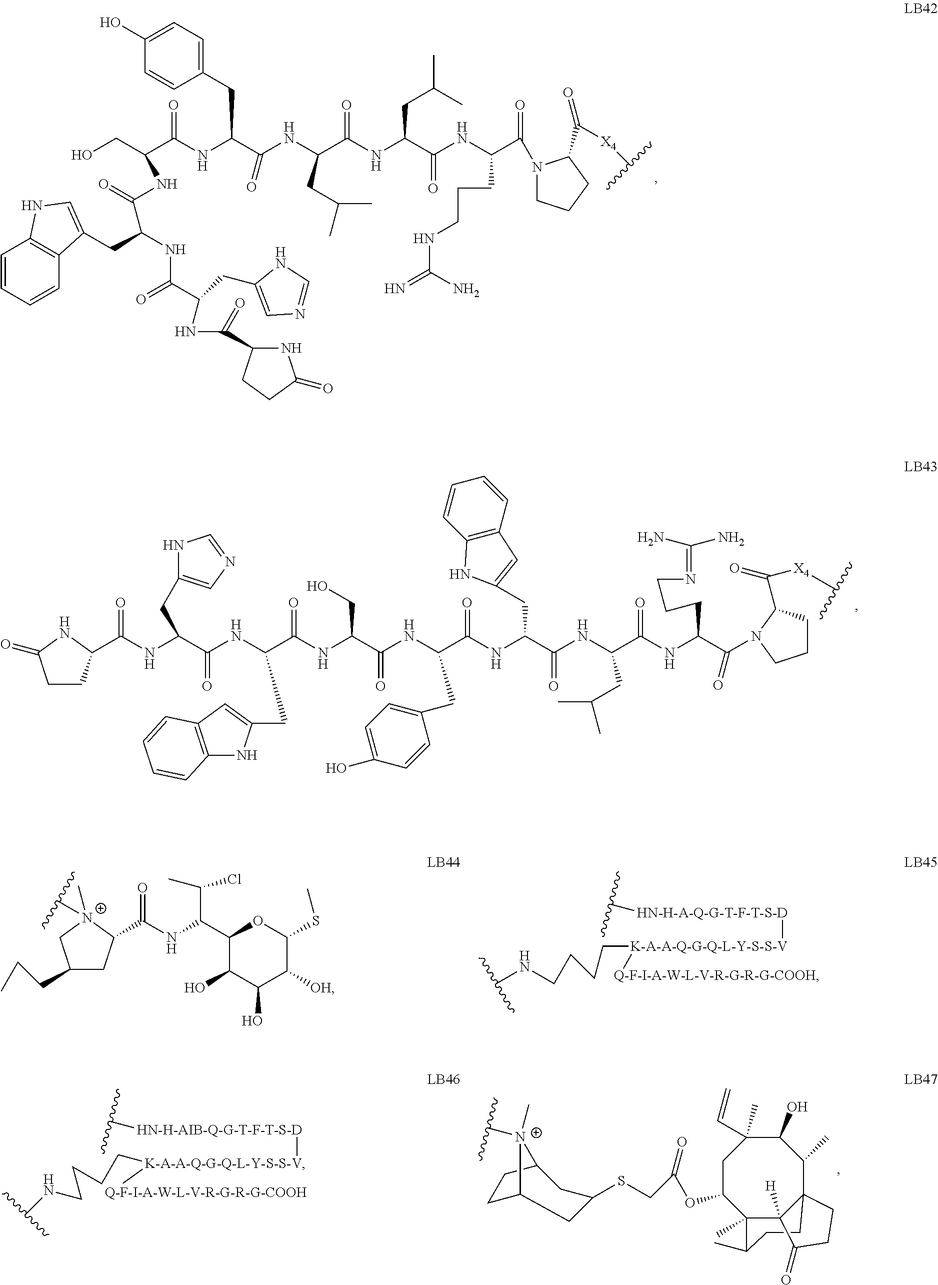 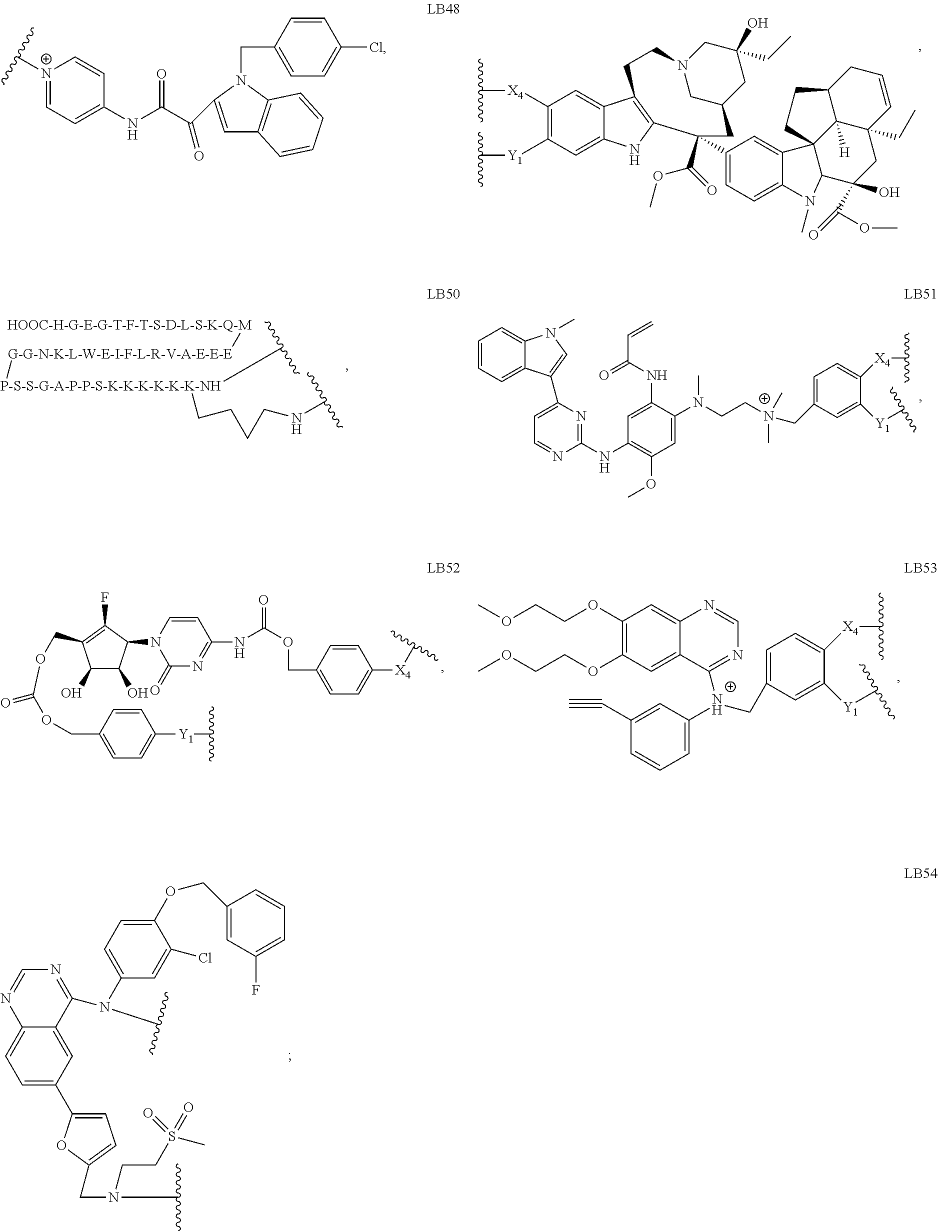
or hydrated salts; or the polymorphic crystalline structures of these compounds; or the optical isomers, racemates, diastereomers or enantiomers; [symbol] is a linkage site that links to L₁ and/or L₂ independently.

36. The antibody drug conjugate according to claim 1, wherein the linker component, L₁, L₂, La₁, La₂, Lb₁, Lb₂, Lc₁ and Lc₂, independently contain:
(a). a self-immolative linker component having one of the following structures: 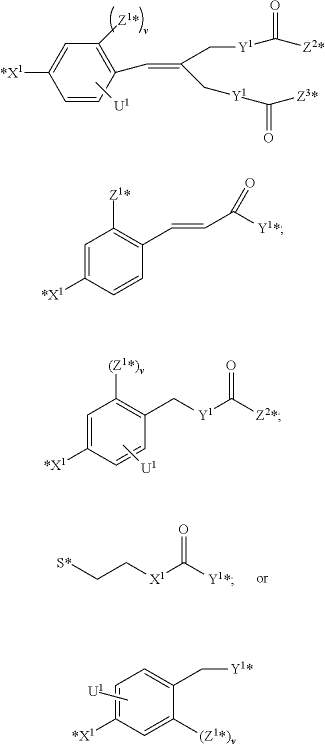
wherein the (*) atom is the point of attachment of additional spacer or releasable linker units, or the cytotoxic agent, and/or the antibody; X₁, Y₁, Z₂ and Z₃ are independently NH, O, or S; Z₁ is independently H, NH, O or S; v is 0 or 1; U1 is independently H, OH, C₁˜C₆ alkyl, (OCH₂CH₂)ₙF, C₁, Br, I, OR₅, SR₅, NR₅R₅′, N═NR₅, N=R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′) wherein R₅ and R₅′ are as defined above;
(b). a non-self-immolative linker component having one of the following structures: 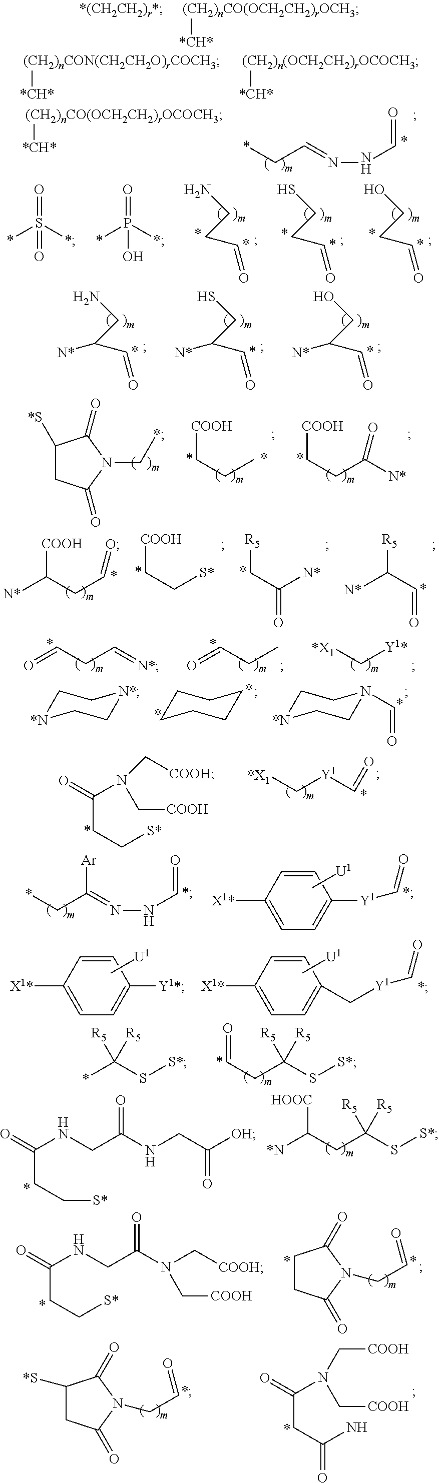 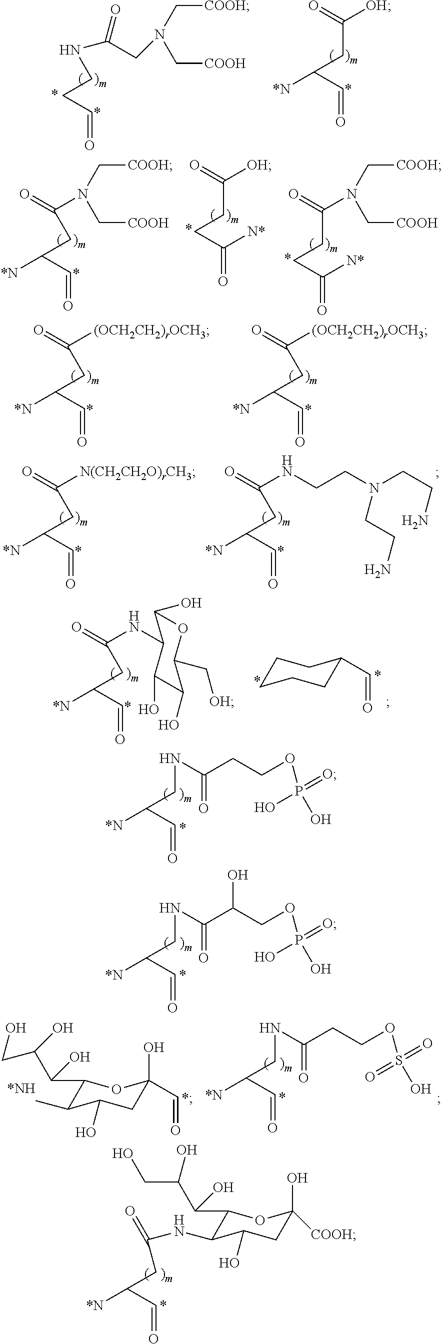 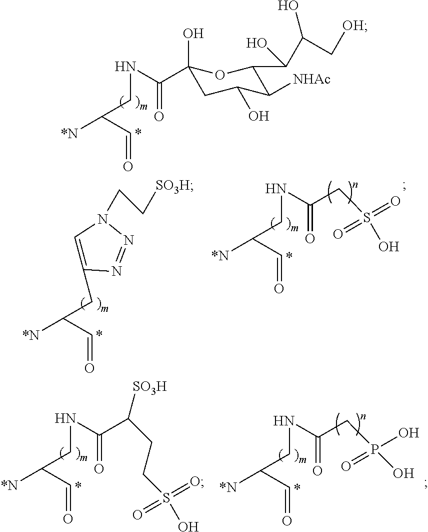
wherein the (*) atom is the point of attachment of additional spacer R₁ or releasable linkers, the cytotoxic agents, and/or the binding molecules; X₁, Y₁, U1, R₁, R₅, R₅′ are defined as above; r is 0˜100;
m and n are 0˜6 independently;
(c). one or more linker components of 6-maleimidocaproyl (“MC”), maleimidopropanoyl (“MP”), valine-citrulline (“val-cit” or “vc”), alanine-phenylalanine (“ala-phe” or “af”), p-amino-benzyloxycarbonyl (“PAB”), 4-thiopentanoate (“SPP”), 4-(N-maleimidomethyl)-cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), or natural or unnatural peptides having 1˜8 natural or unnatural amino acid unites, wherein lysine, glutamic acid, aspartic acid, cysteine, or tyrosine may contain a side chain polyethyleneoxy unit of formula (OCH₂CH₂)ₚOR₃, (OCH₂CH(CH₃))ₚOR₃, NH(CH₂CH₂O)ₚR₃, C(O)(CH₂CH₂O)ₚR₃, C(O)(CH₂CH₂OCH₂CH)ₚR₃, NH(CH₂CH(CH₃)O)ₚR₃, C(O)(CH₂OCH₂CH)ₚR₃, N[(CH₂CH₂O)ₚR₃][(CH₂CH₂O)_{p′}R_{3′}], (OCH₂CH₂)ₚCOOR₃, or CH₂CH₂(OCH₂CH₂)ₚCOOR₃, NH(CH₂CH₂O)ₚCH₂CH₂R₃, wherein p and p′ are independently an integer selected from 0 to about 100, or combination thereof; wherein R₃ and R_{3′} are independently H, OH, NH₂, N(CH₂)₂, N(C₂H₅)₂, N(C₃H₇), C(═O)H, C(═O)CH₃, C₁-C₈ of alkyl, NH(C₄˜C₇ glycoside) or NH(C₄˜C₇ glycoside) 2; or combination above thereof;
(d). one or several of the following structure units: 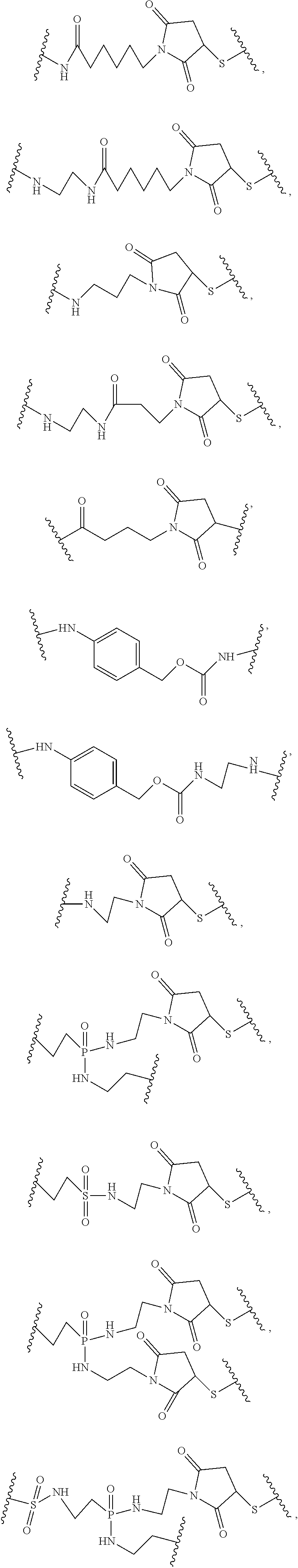 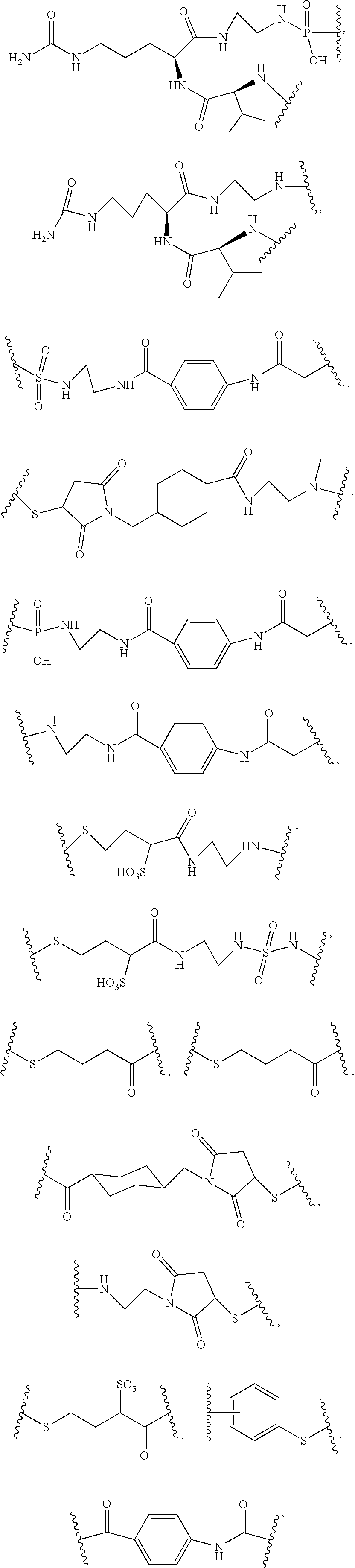 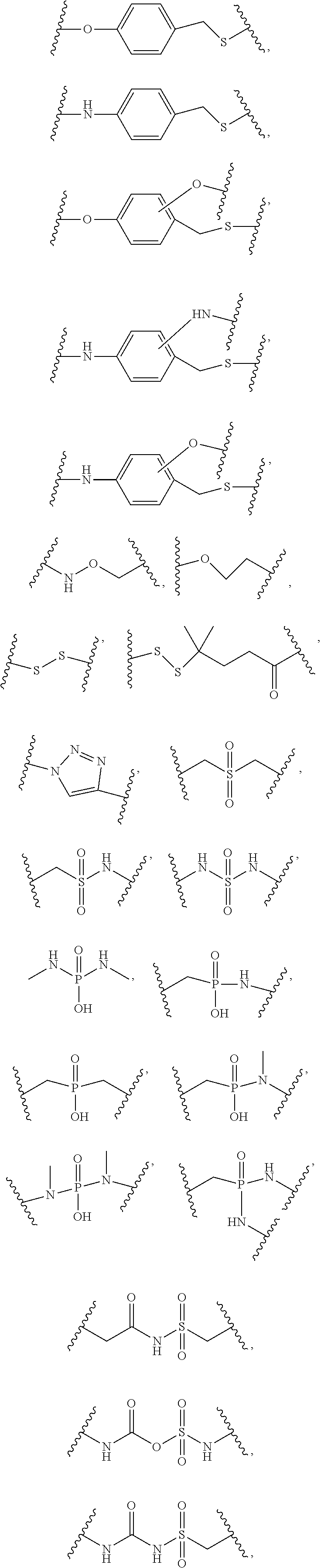 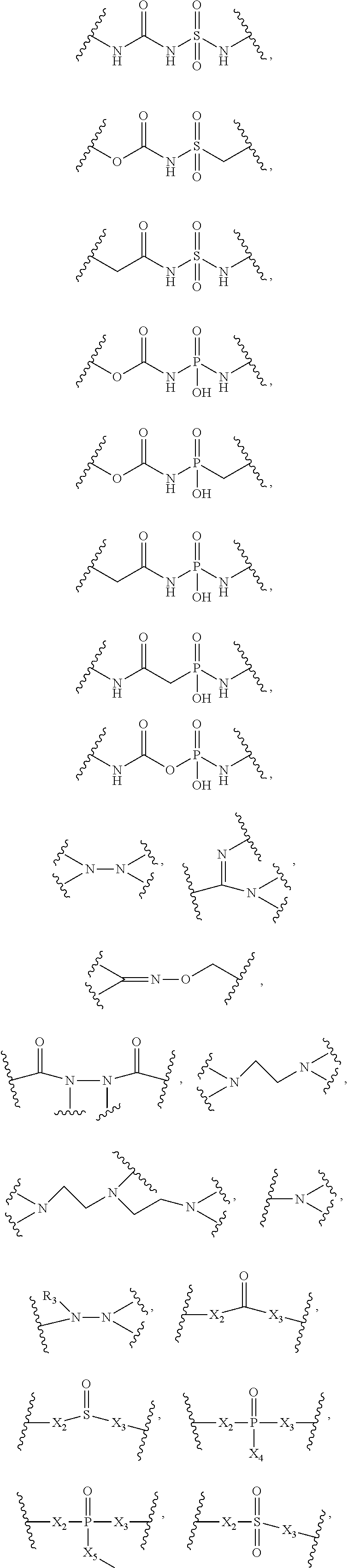 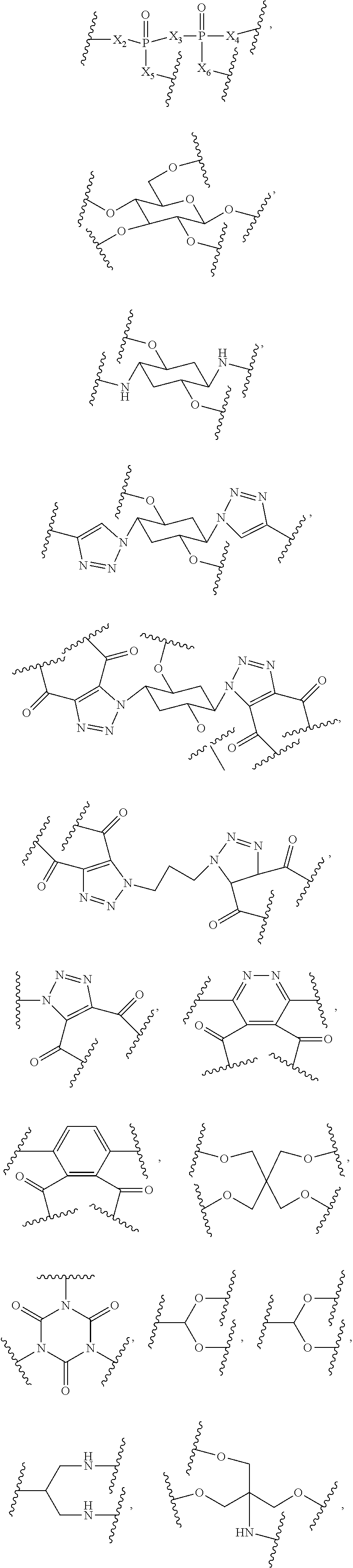 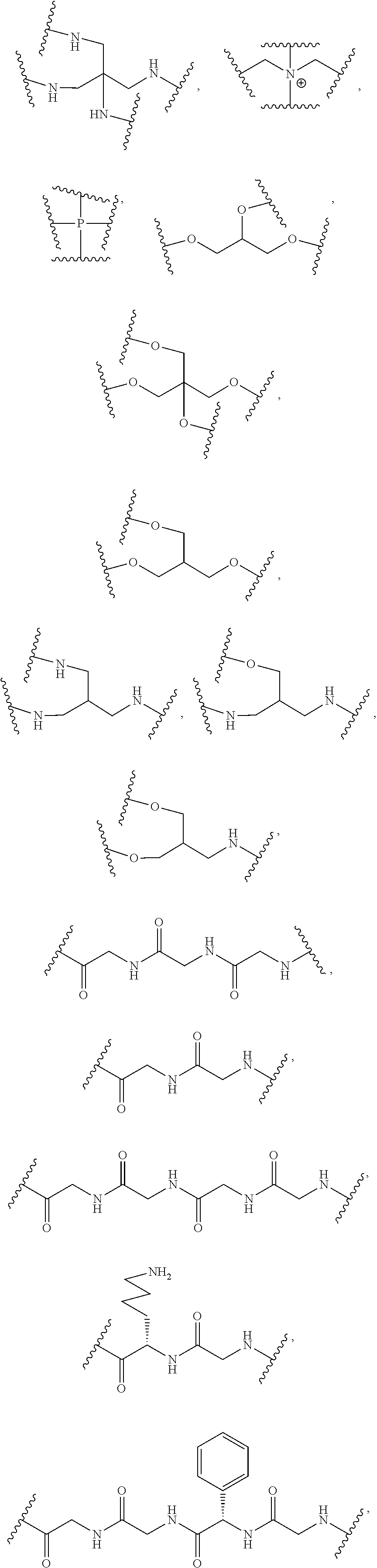 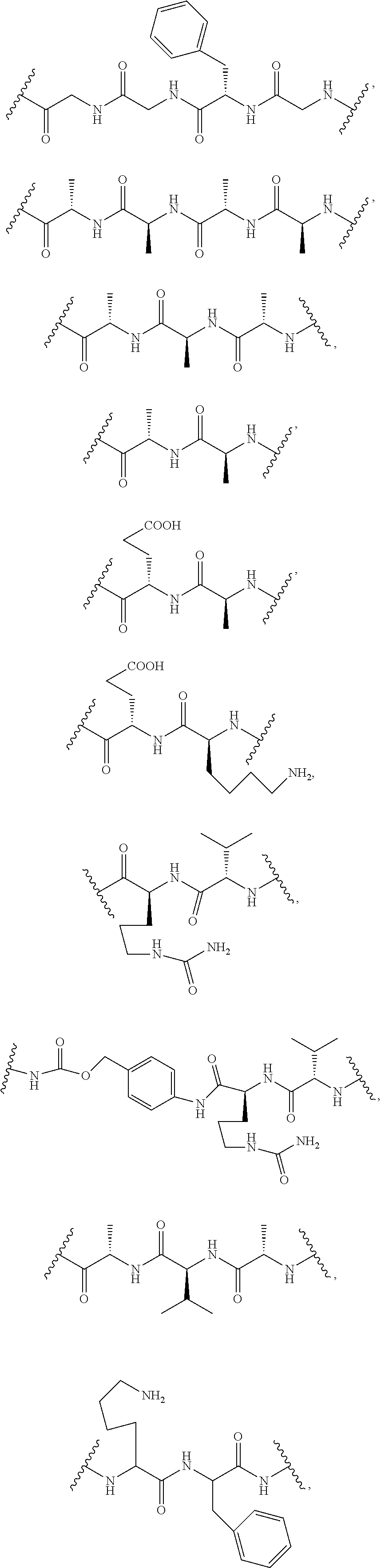 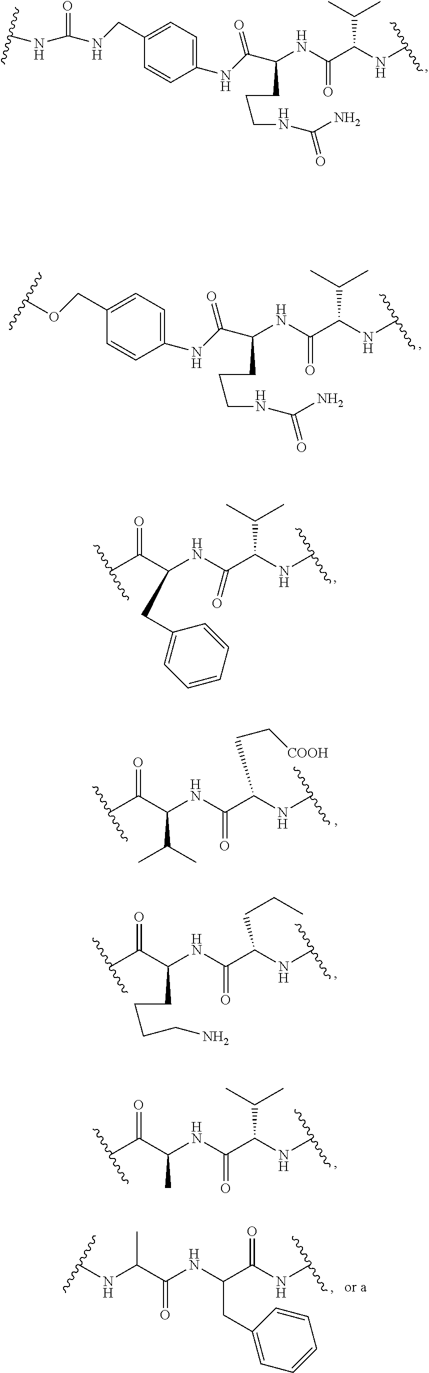
combination above thereof; wherein [symbol] is the site of linkage; X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1˜8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 100.

37. The antibody drug conjugate according to claim 1, wherein the structure of 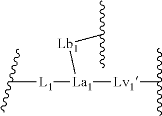
in formula (I) is further having the structure of formula (Ia);
wherein the structure of 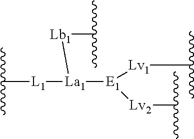
in formula (II) is further having the structure of formula (Ib) or (Ic);
wherein the structure of 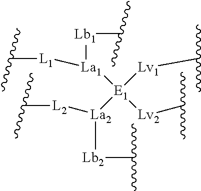
in formula (III) is further having the structure of formula (Id), (Ie), (If) or (Ig), or 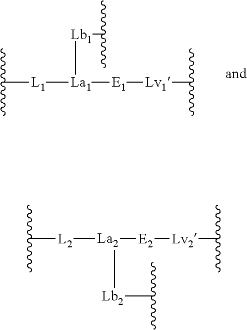
in formula (IV) is further having the structure of formula (Ia), wherein said (Ia), (Ib), (Ic), (Id), (Ie), (If) and (Ig) are illustrated as following: 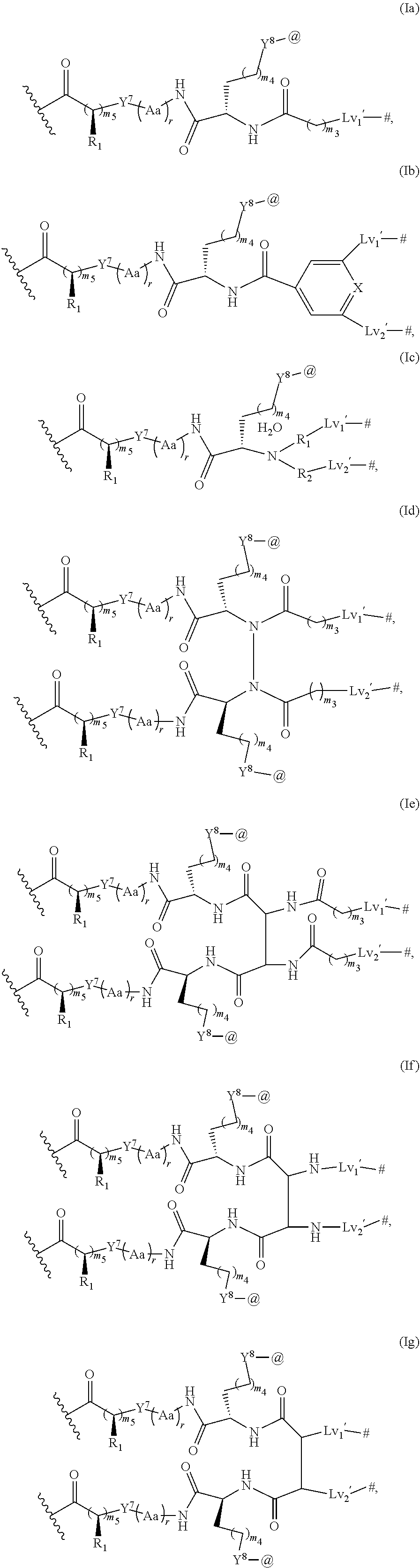
wherein “ [symbol] ” is a site that links a drug; “#” is a site that links a S (thiol), O (phenol), NH (amino), CHO (aldehyde), C(═O) (ketone), C(O)(NH) (amide) and C(O)(OH) (carboxylate) of an antibody; Aa is L- or D-natural or unnatural amino acids; “@” is a site that links Lc₁ or Lc₂ described in the formula (I), (II) and (III) in claim 1;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0, 1, 2, 3, 4, 5, 6, 7, or 8; when r is not 0, (Aa) r is the same or different amino acids or peptide units; when r=0, (Aa) r is absent;
m₁=1-18 (0, 1,2, 3,4,5,6, . . . , 18); m₂=1-100 (1, 2, 3, 4, 5,6, 7,8, . . . , 100); m₃ or m₅=1-8 (1, 2, 3, 4, 5, 6, 7, or 8); m₄=0-8 (0, 1, 2, 3, 4, 5, 6, 7, or 8);
Y₇ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH)O, NHP(O)(OH)O, OP(O)(OH)NH, S, O, OP(O)(OH)OP(O)(OH)NH, NHP(O)(OH)OP(O)(OH)NH, NHP(O)(OH)OP(O)(OH)O, OCH₂CH₂O, OCH₂CH₂NH, N(CH₂CH₂)₂N, NHC₆H₄NH, CH₂;
Y₈ is NHC(═O), NHS(O₂), NH(SO), NHS(O₂)NH, NHP(O)(OH)NH, C(O)NH, OC(O)NH, NHC(O)NH, C(O), N, NH, CH₂, or CH;
Lv₁′ and Lv₂′ are independently or jointly selected from: 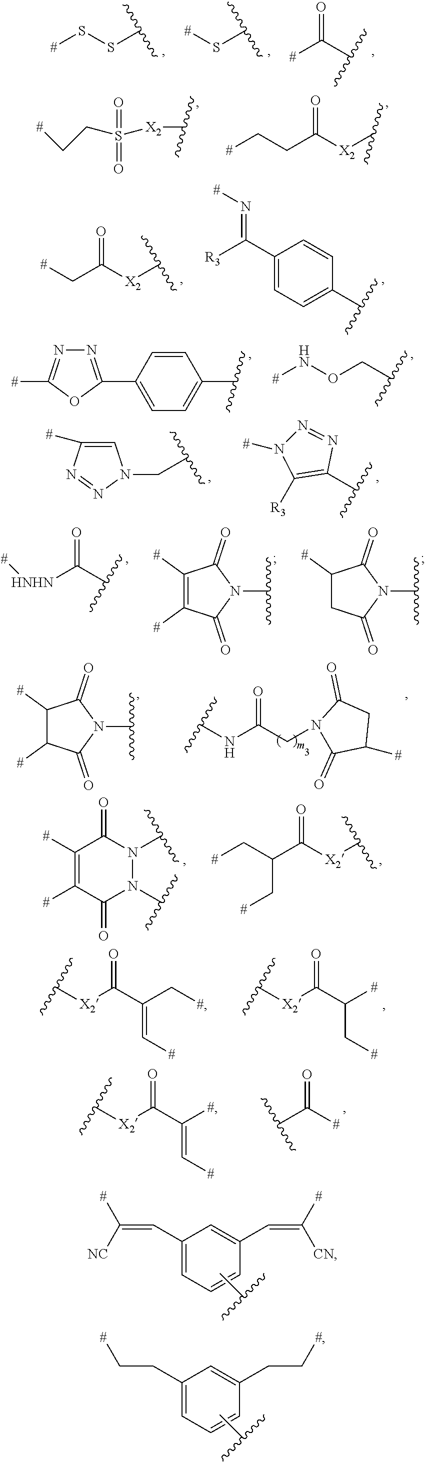 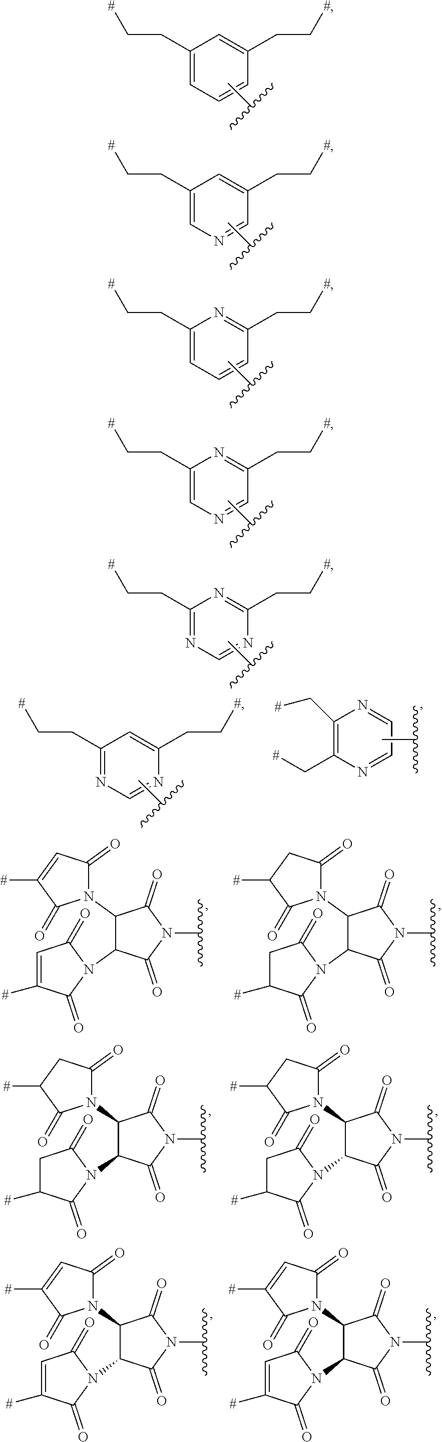 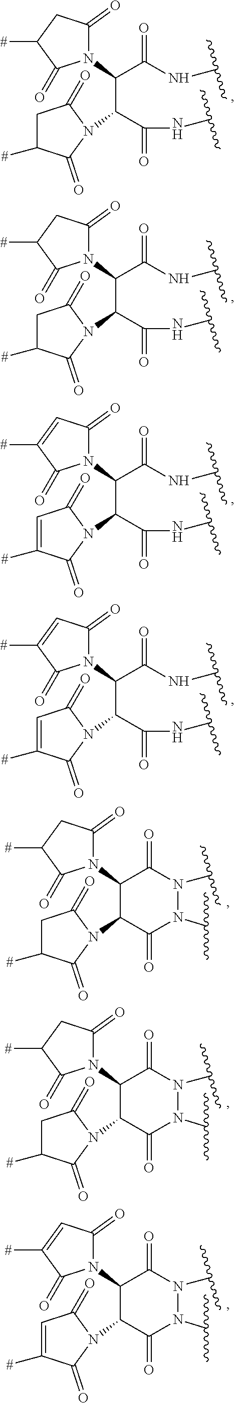 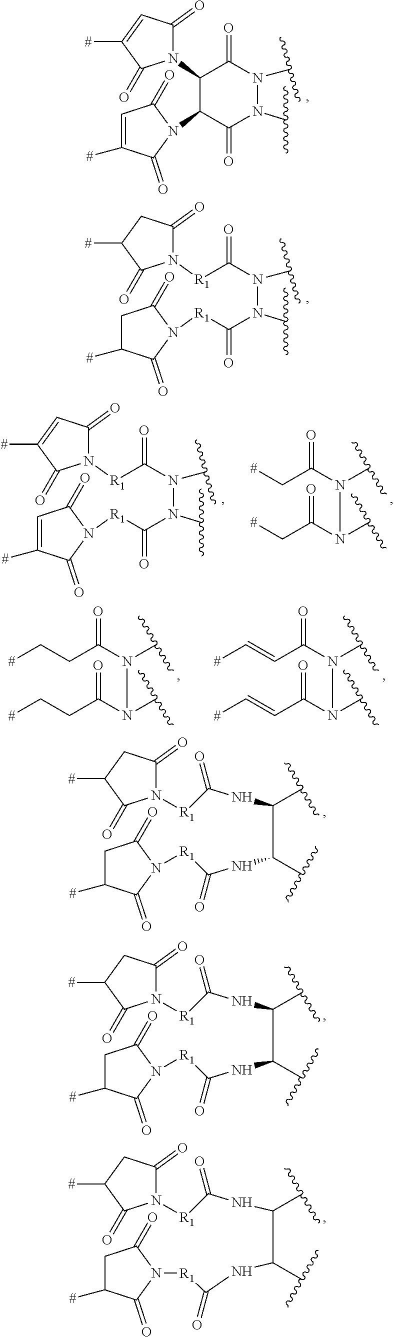 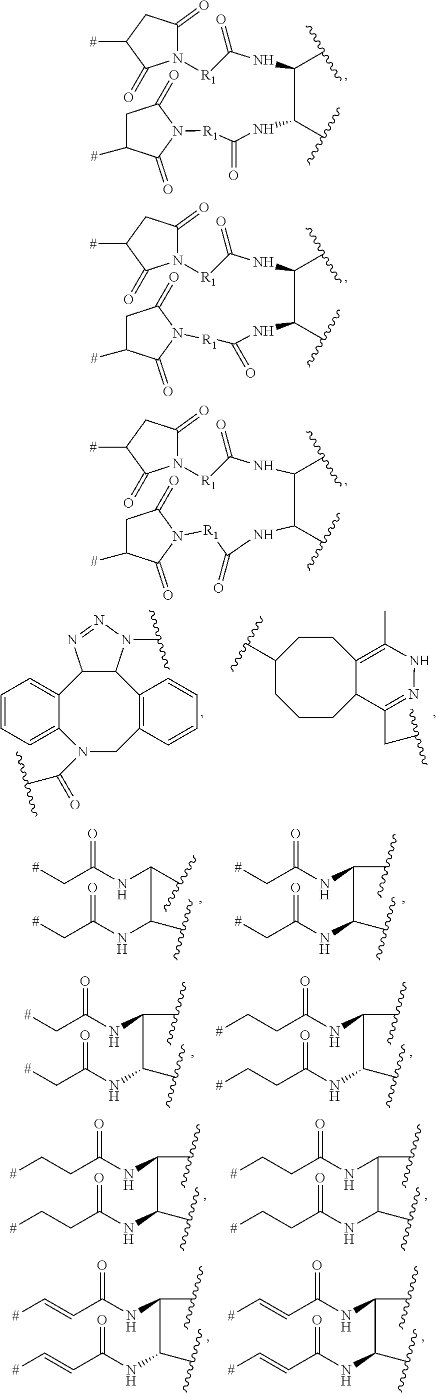 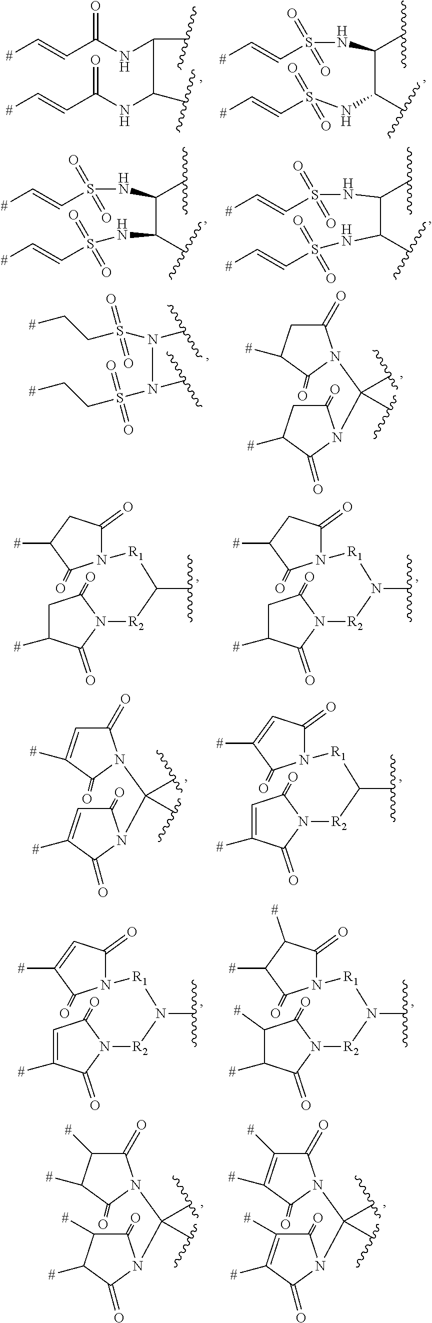 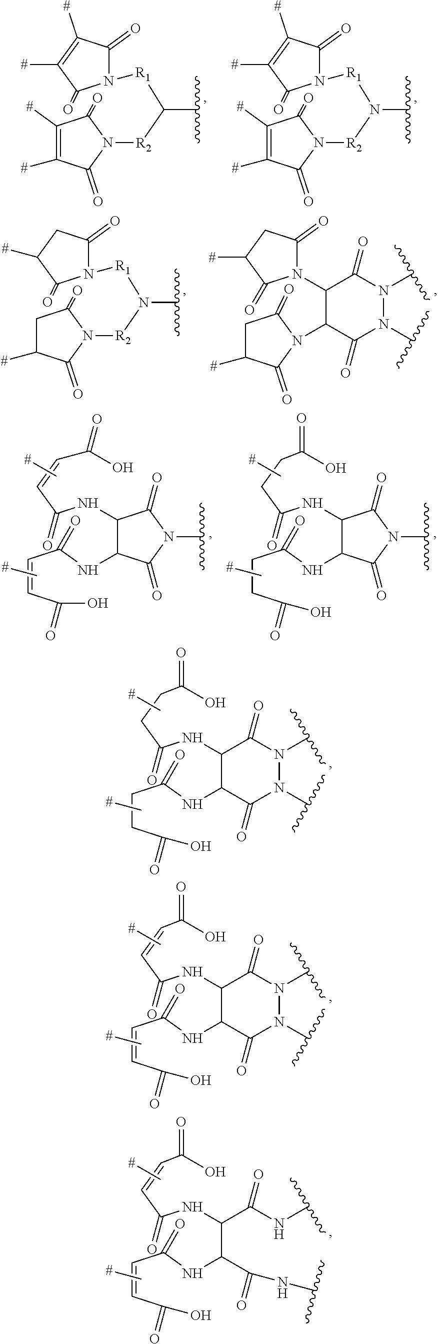 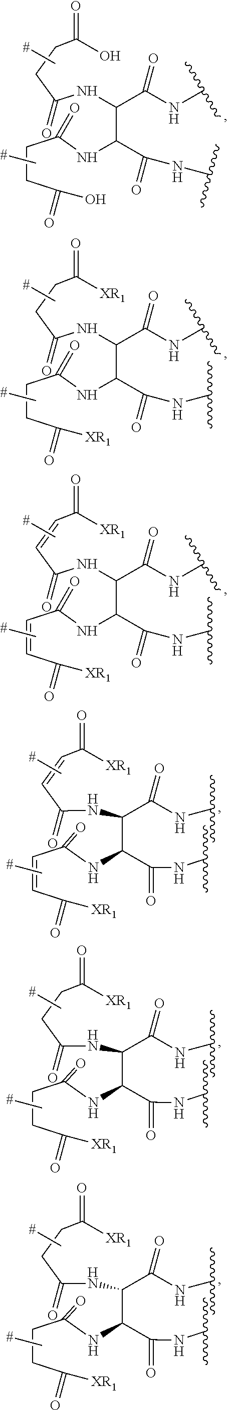 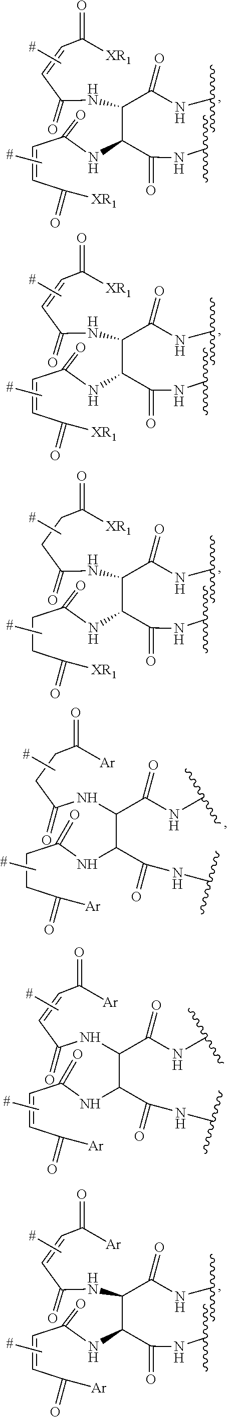 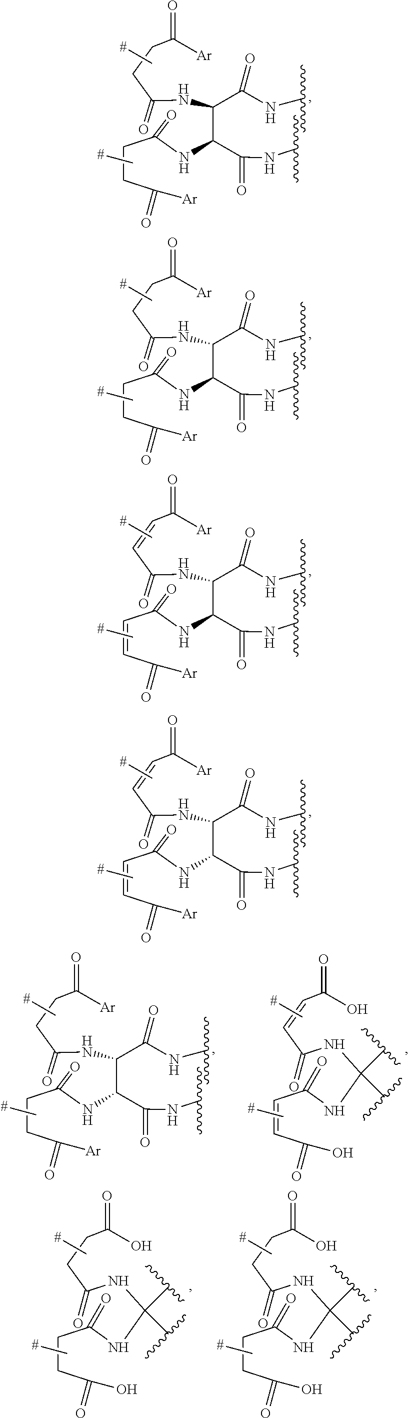 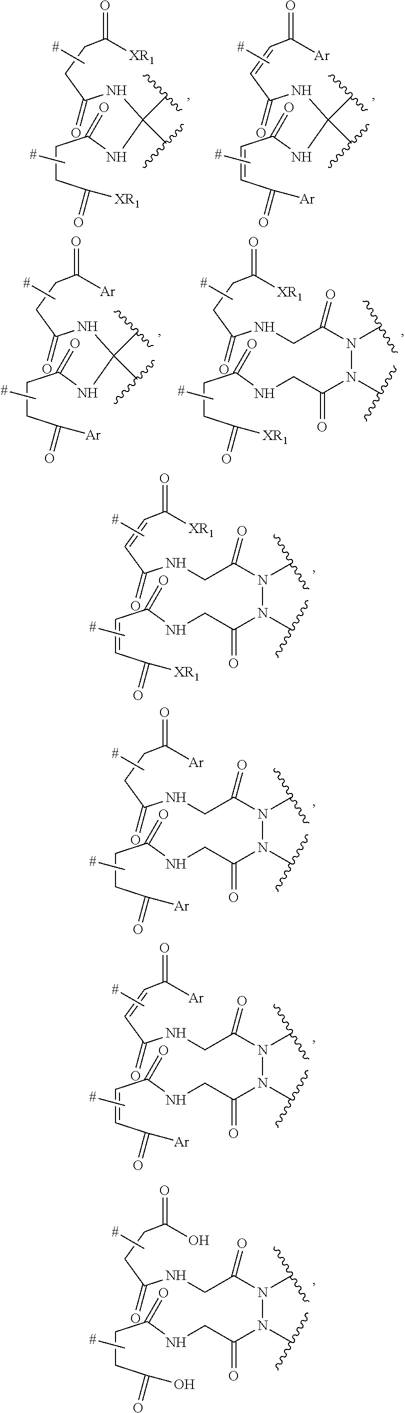 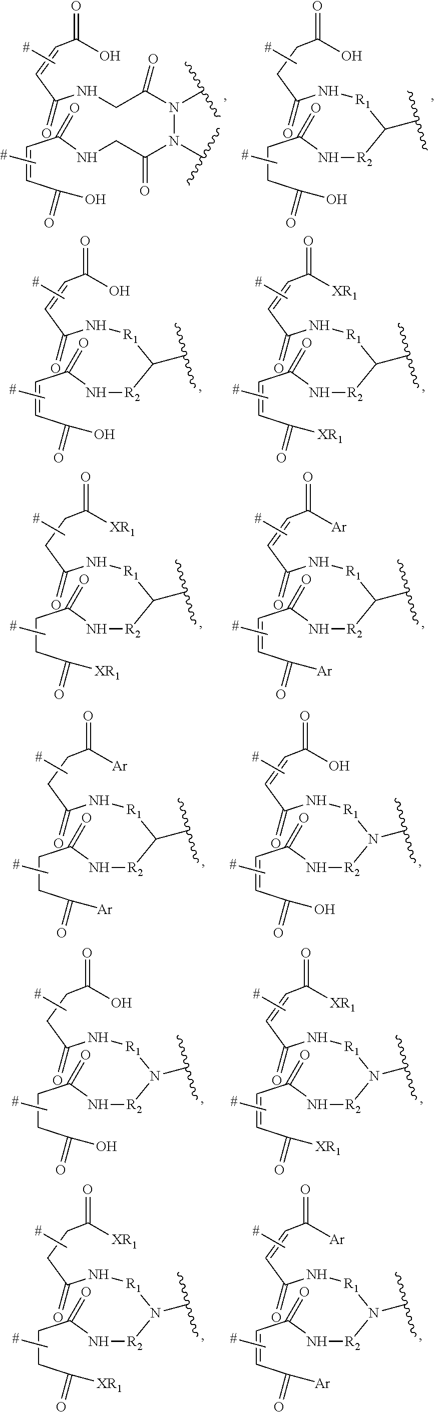 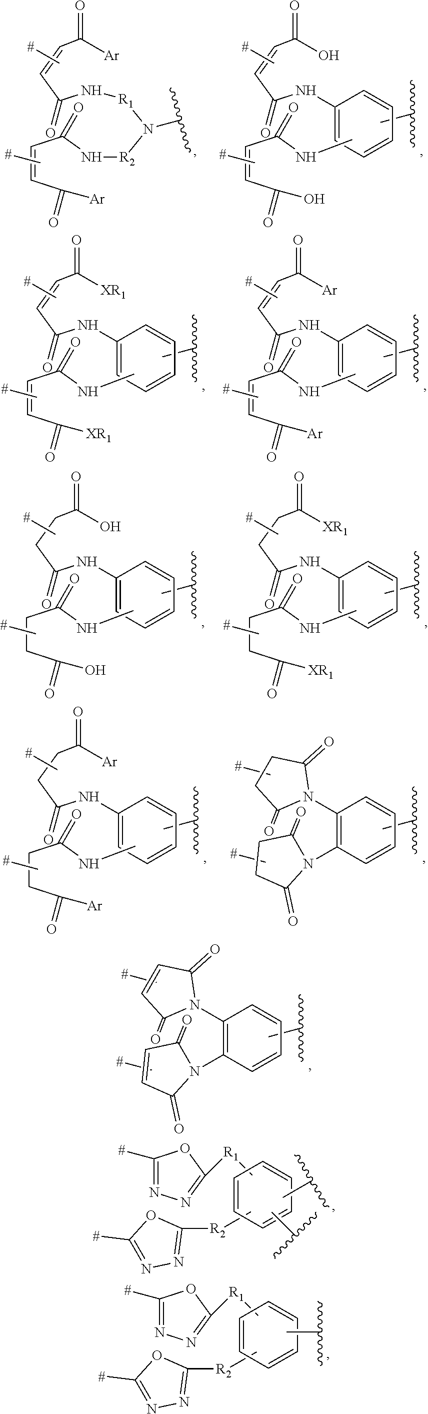 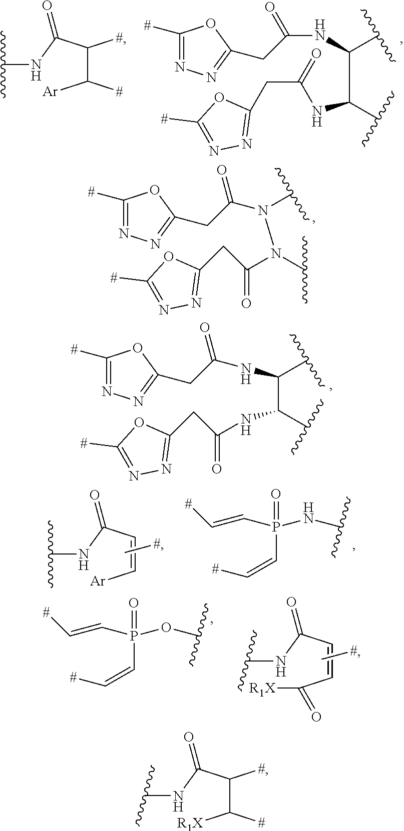
wherein “ [symbol] ” is a site that links to the linker component; “#” is the site as Lv₁′ and Lv₂′ indicated in the formulas that links a S (thiol), O (phenol), NH (amino), CHO (aldehyde), C(═O) (ketone), C(O)(NH) (amide) and C(O)(OH) (carboxylate) of an antibody; wherein R₁, m3, X₁′ and X₂′ are described above; X is O, NH, S, CH₂; the connecting bond “—” in the middle of the two atoms means it can link either one of the two atoms; Ar is an aromatic group.

38. The antibody drug conjugate according to claim 1, the core linker structure (L₁″) having an affinity ligand in formula (I): 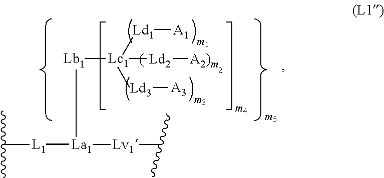
wherein (1.1″) is further selected from Formula (Ta′); 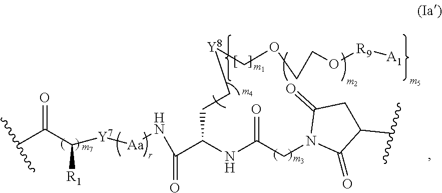
wherein Aa is L- or D-natural or unnatural amino acids; A₁ is the affinity ligand defined the same in claim 1;
R₁ is H, C₁-C₈ alkyl, OH, CH₂OH, CH₂CH₂OH, NH₂, SH, SCH₃, CH₂COOH, CH₂CH₂COOH, CH₂CH₂CH₂CH₂NH₂, C₆H₅, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂;
r is 0, 1, 2, 3, 4, 5, 6, 7, or 8; when r is not 0, (Aa) r is the same or different amino acids or peptide units; when r=0, (Aa) r is absent;
m₁=1-18 (0, 1, 2, 3, 4, 5, 6, . . . , 18); m₂=1-100 (1, 2, 3, 4, 5, 6, 7, 8, . . . , 100); m₃ or m₅=1-8 (1, 2, 3, 4, 5, 6, 7, or 8); m₄=0-8 (0, 1, 2, 3, 4, 5, 6, 7, or 8);
Y₇ is NH, OCH₂NH, NHC(═O), NHNH, C(═O)NH, N(R₁), SO₂, P(O)(OH), NHS(O)₂, NHS(O)₂NH, NHS(O)₂NHC(O), NHS(O)₂NHC(O)O, NHS(O)₂NHC(O)NH, NHP(O)(OH), NHP(O)(OH)NH, OP(O)(OH) O, NHP(O)(OH) O, OP(O)(OH)NH, S, O, OP(O)(OH) OP(O)(OH)NH, NHP(O)(OH) OP(O)(OH)NH, NHP(O)(OH) OP(O)(OH)O, OCH₂CH₂O, OCH₂CH₂NH, N(CH₂CH₂)₂N, NHC6H₄NH, CH₂;
Y₈ is NHC(═O), NH, O, NHS(O₂), NH(SO), NHS(O₂)NH, NHP(O)(OH)NH, 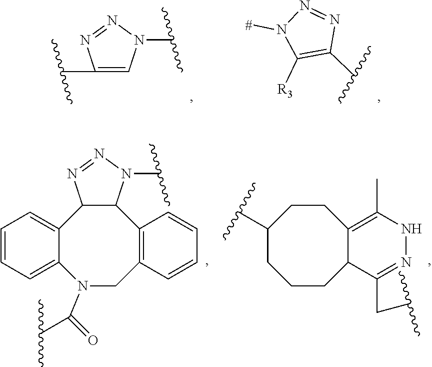
C(O)O, C(O), OC(O)NH, C(O)NH, or Ar;
R₉ is (O═)CR₁, (O═)CNHR₁, NHC(═O), NH, O, NHS(O₂), NH(SO), NHS(O₂)NH, NHP(O)(OH)NH or C(O)NH, R₁ (COCH₂NH)ₘ₄H, R₁(Aa)ᵣ, (Aa)r, C(O), Ar or 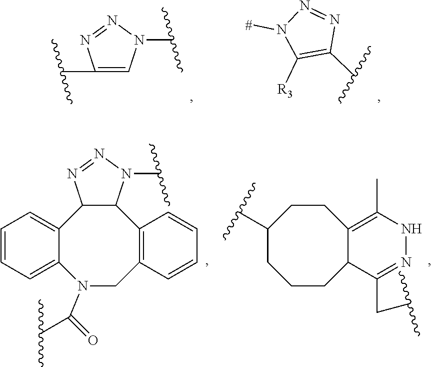
wherein R₃ is H, C₁-C₈ alkyl, ester, amide, Ar, ketone, alkyl acid, alkyl alcohol, alkyl amine, CH₂C₆H₅, CH₂C₆H₄OH, CH(OH)CH₃, CH₂C(O)NH₂, CH₂CH₂C(O)NH₂, CH₂CH₂CH₂NHC(═NH)NH₂; R₁ is defined above.

39. The antibody drug conjugate according to claim 1, the core linker structure (called L₁″ and L₂″ fused jointly) having an affinity ligand in Formula (III): 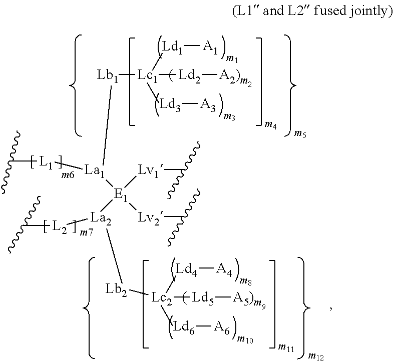
which is further selected from the following formula (Ib) and (Ic): 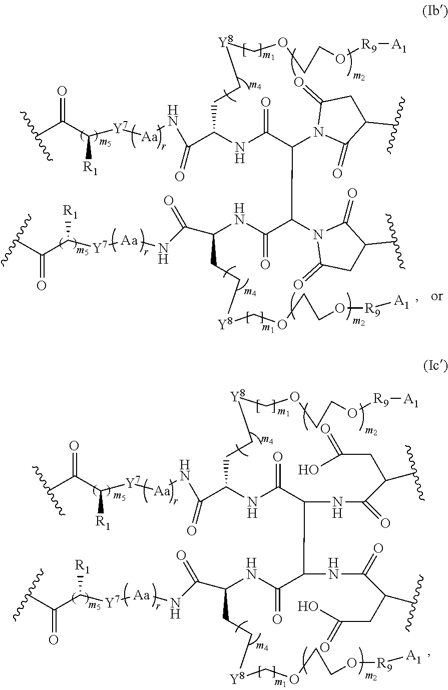
wherein R₁, Y₇, Y₈, R₉, A₁, Aa, r, m₁, m₂, m₄, and m₅ are defined the same.

40. The antibody drug conjugate according to claim 1, the antibody drug conjugate with a branch affinity ligand having one of the following structures: 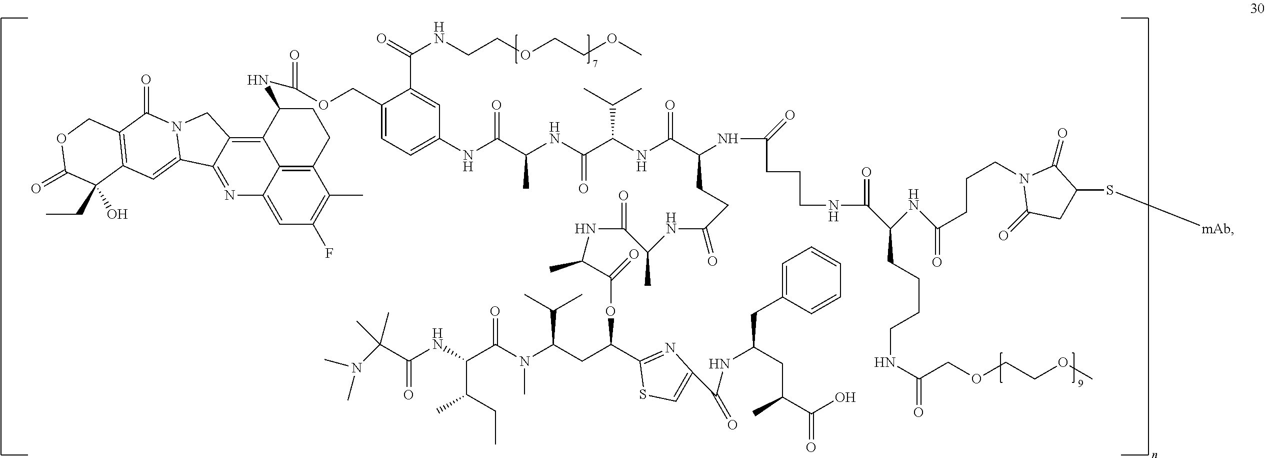 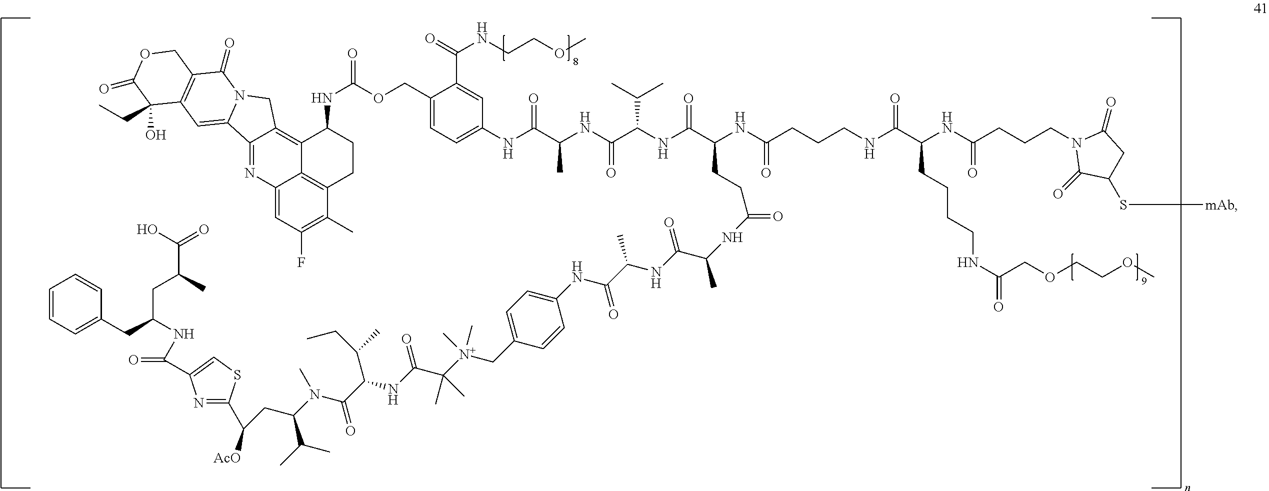 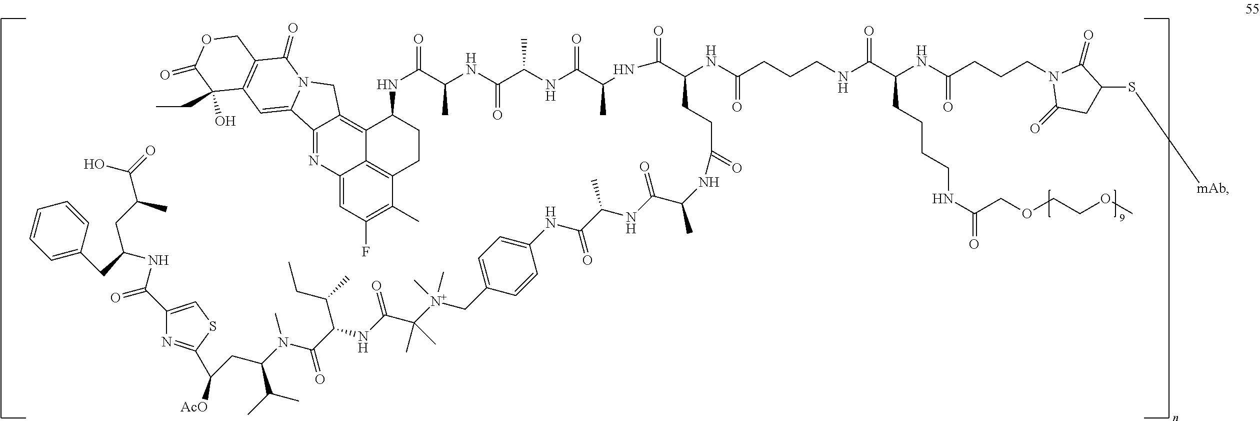 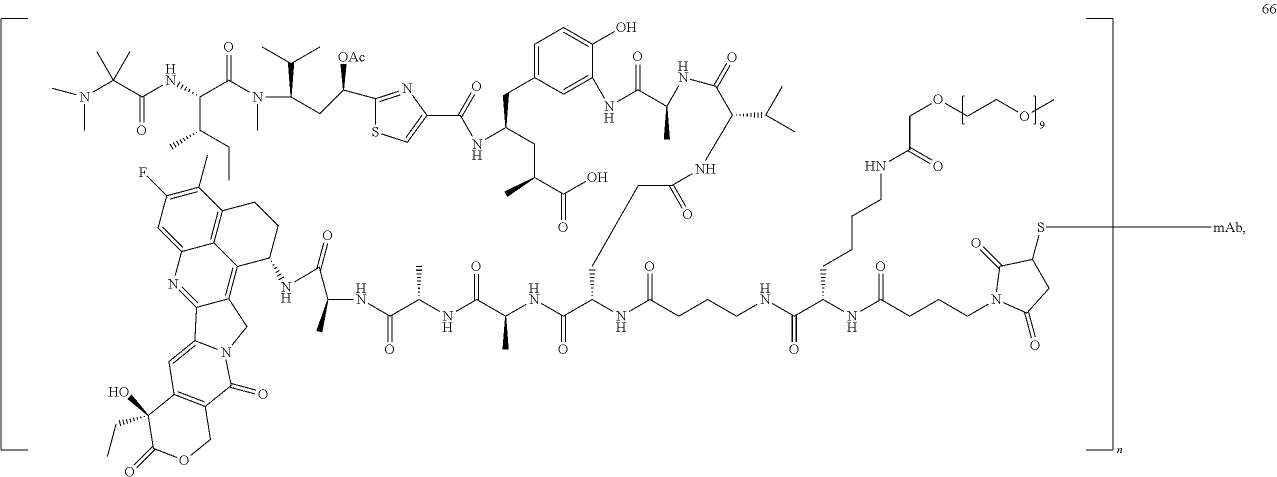 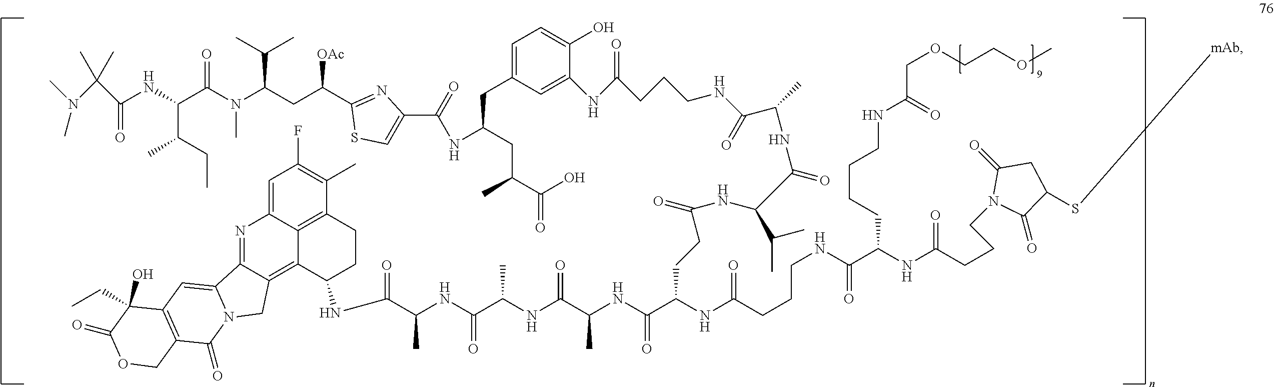 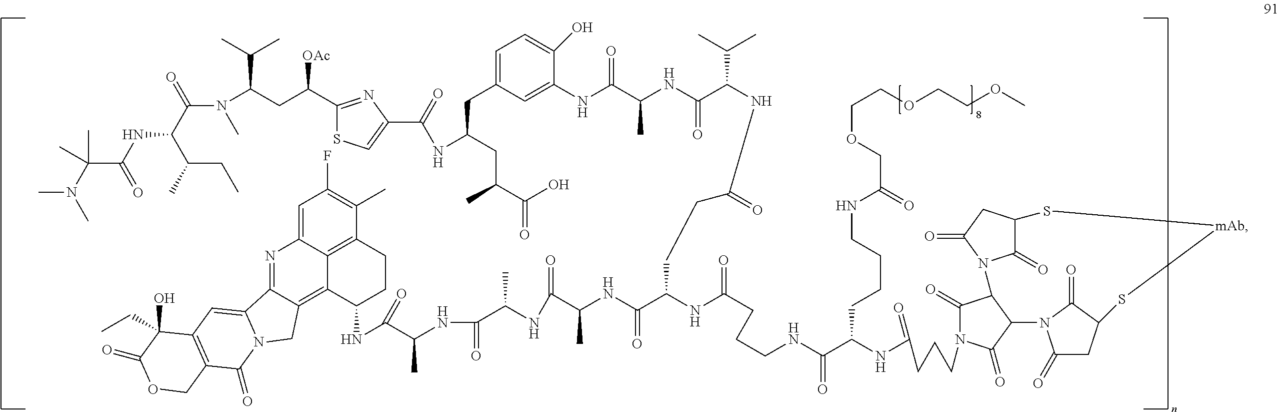 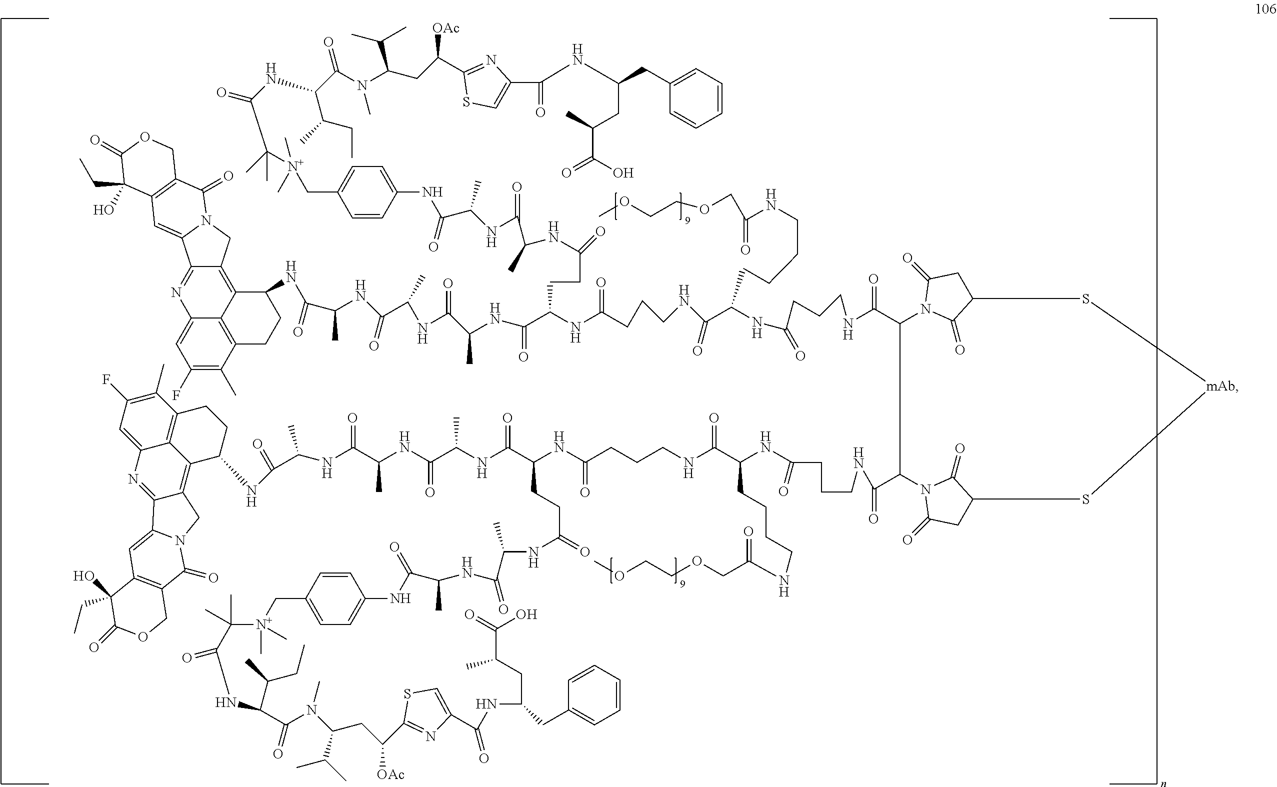 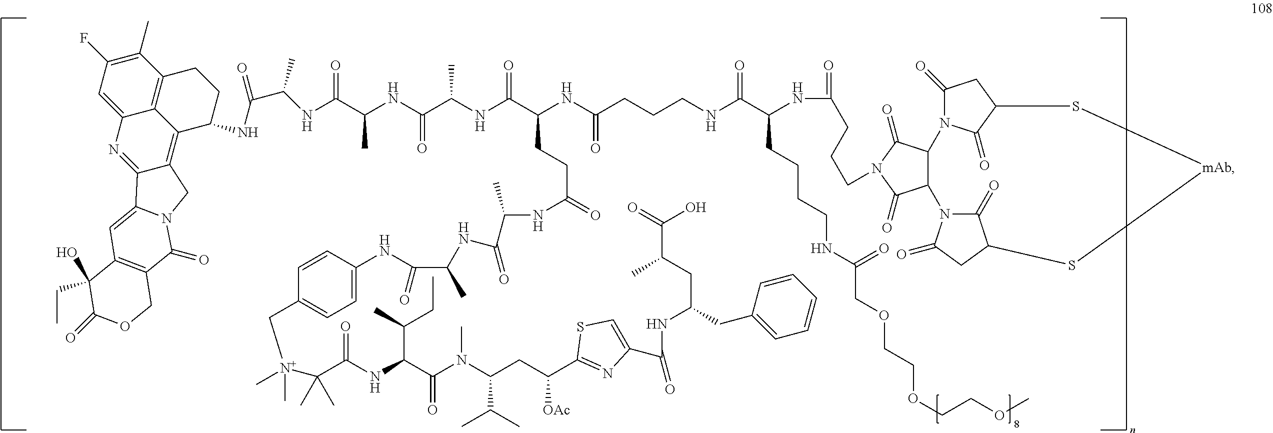 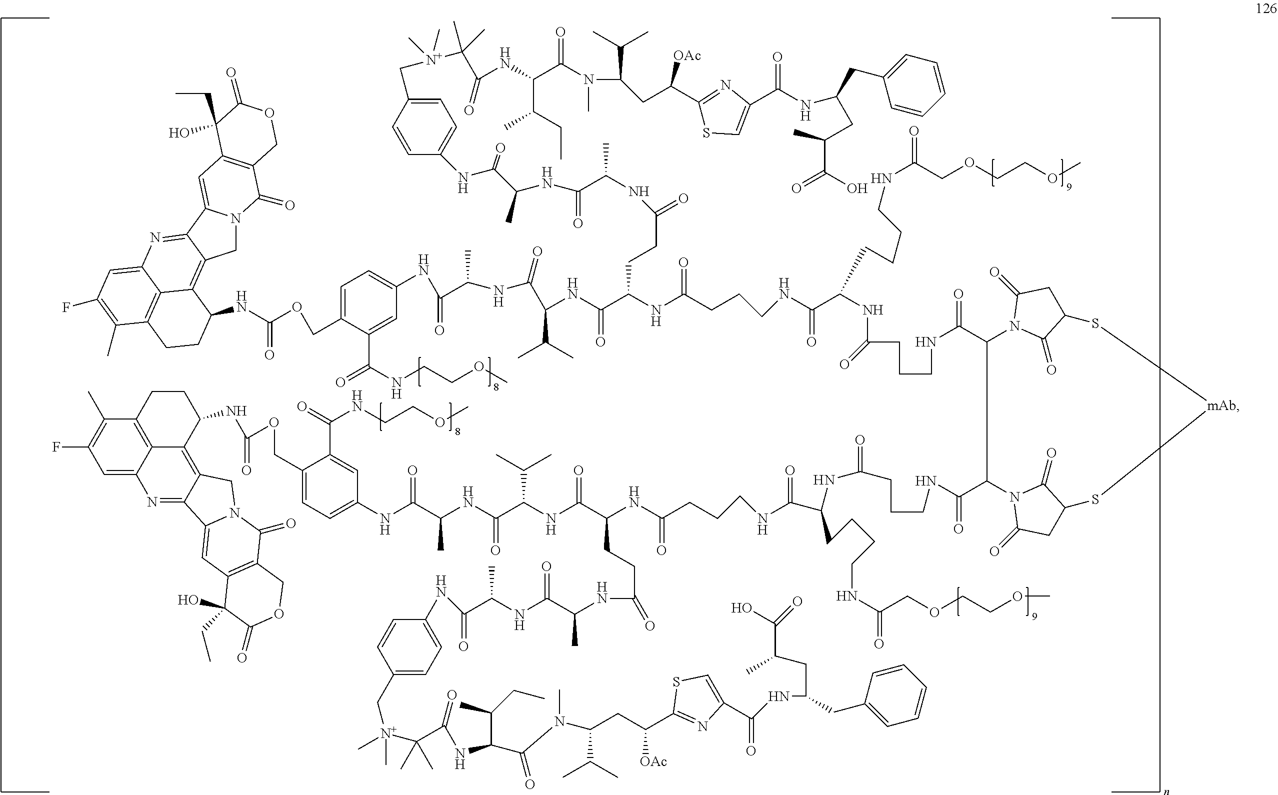 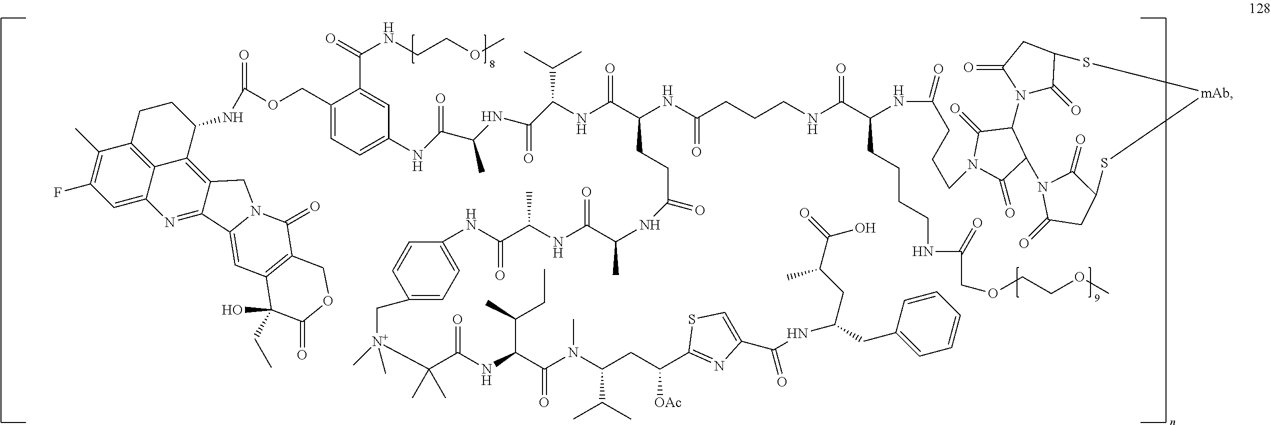 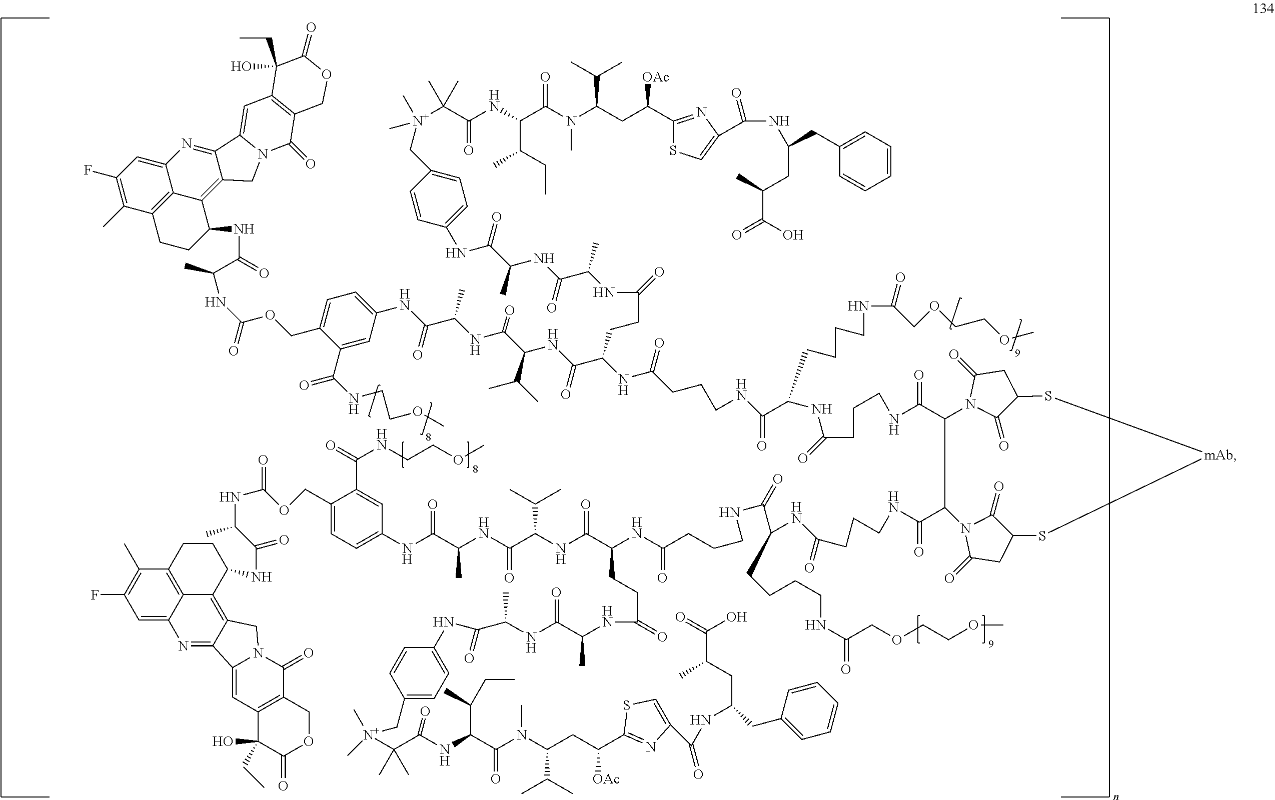 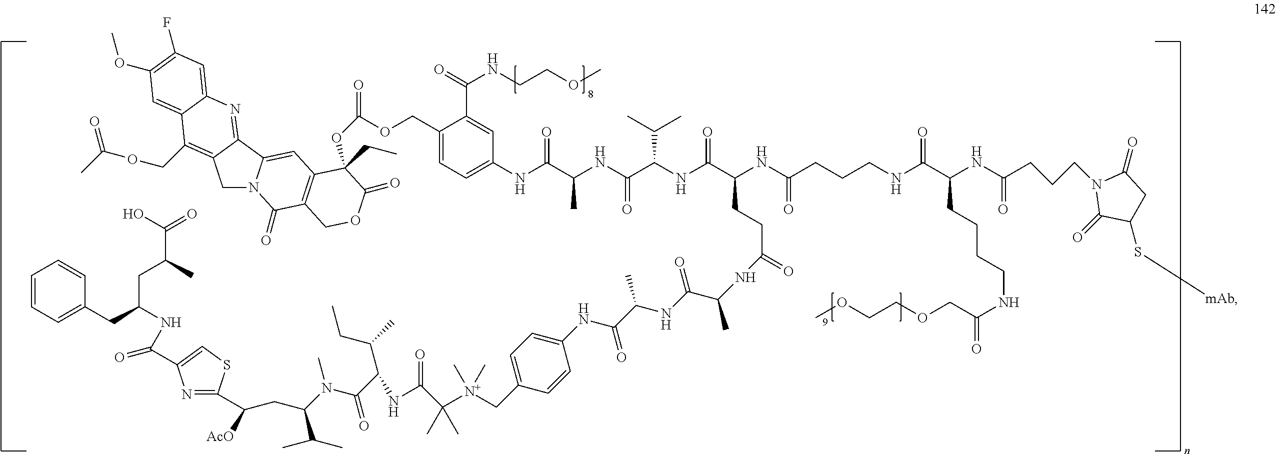 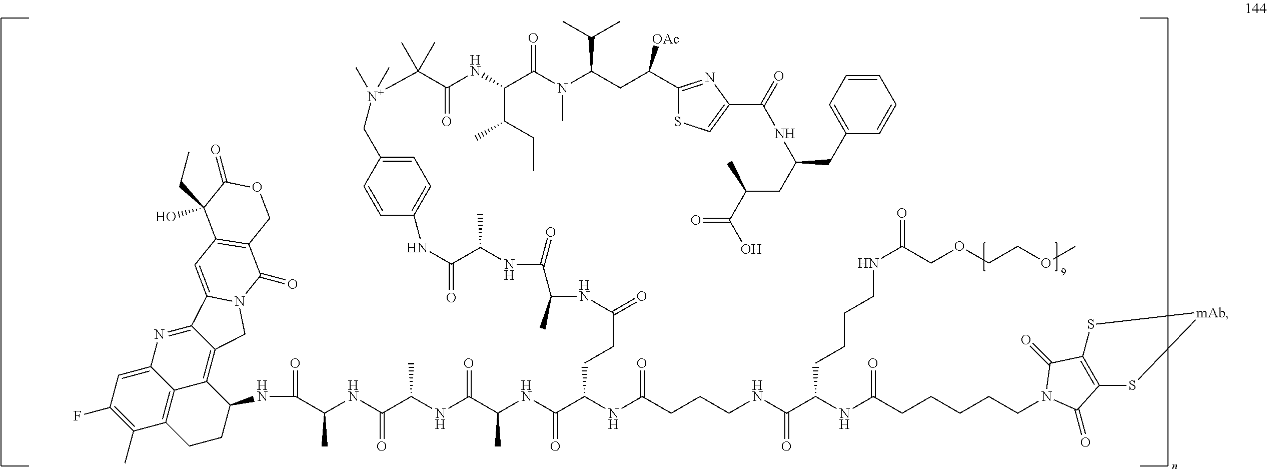 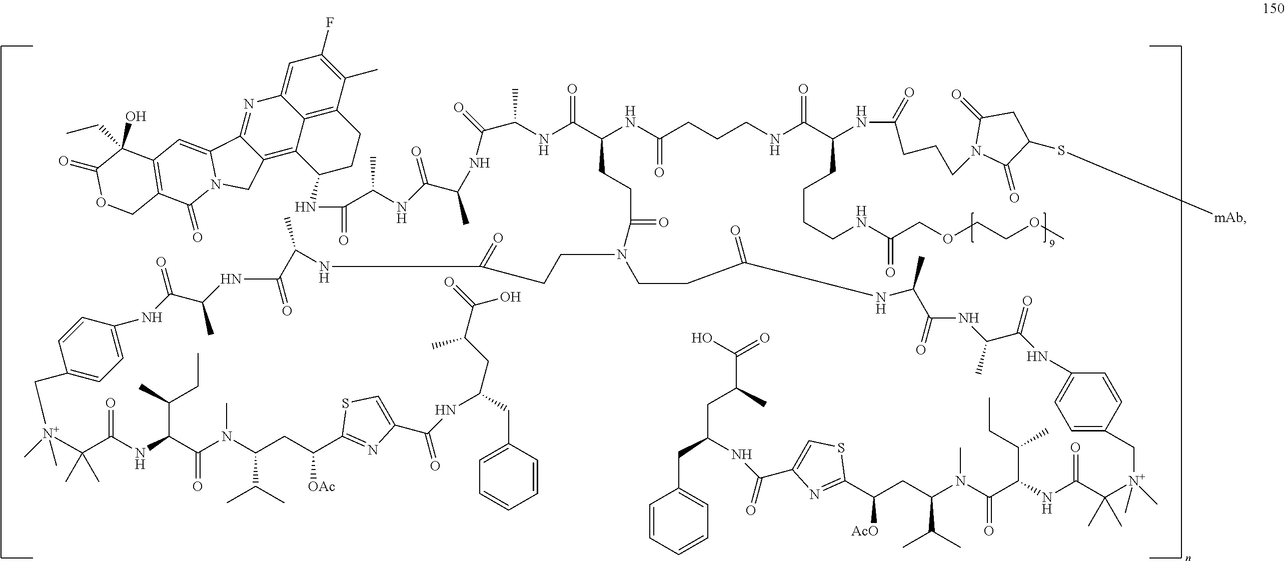 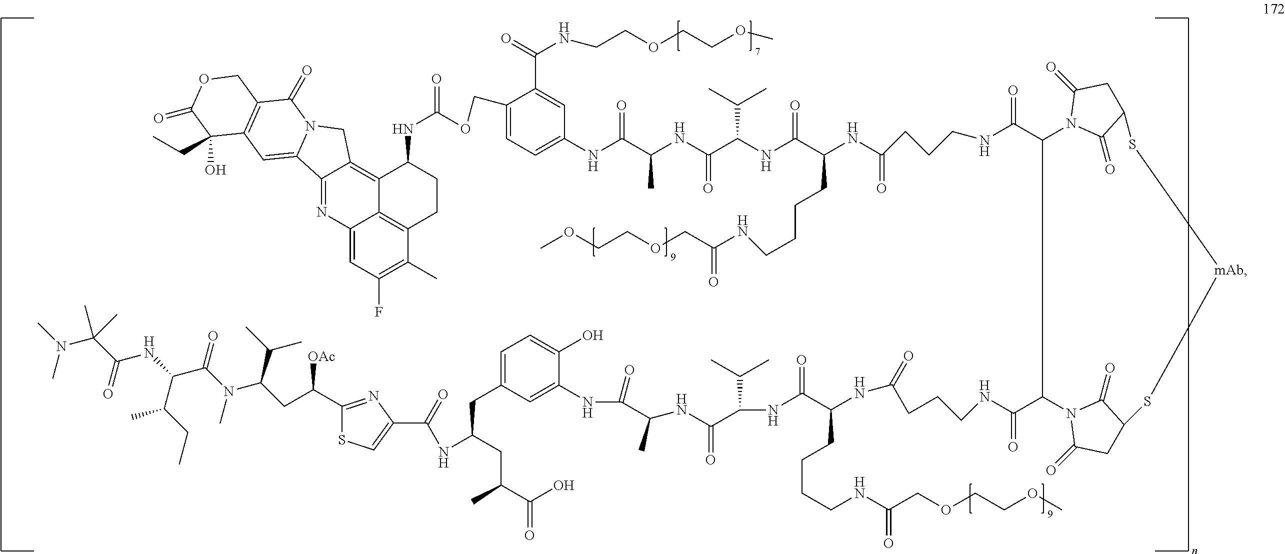 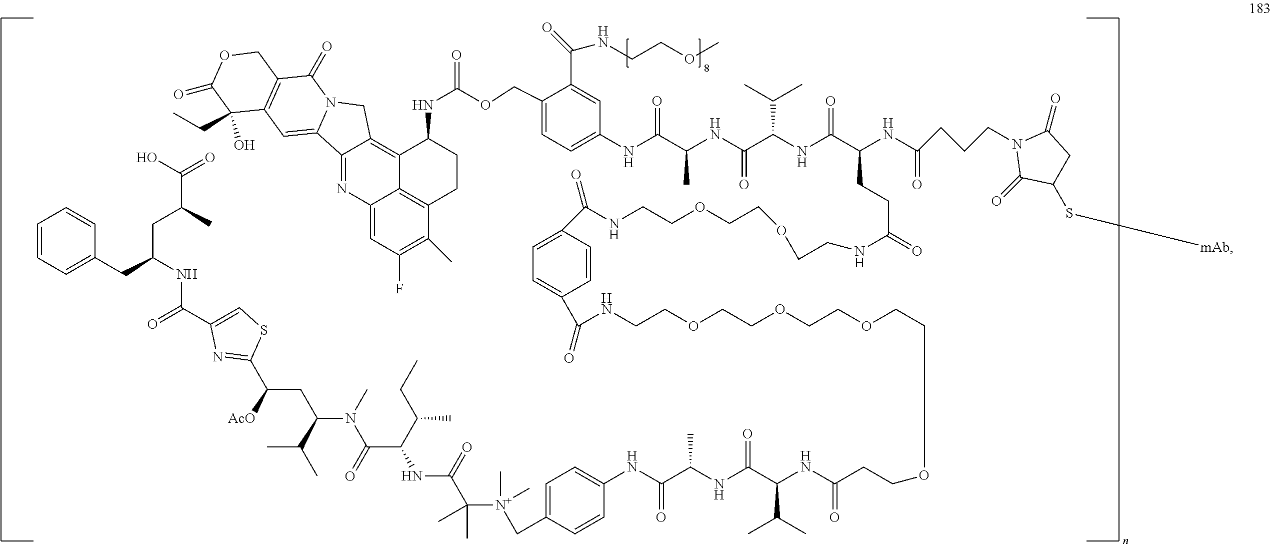 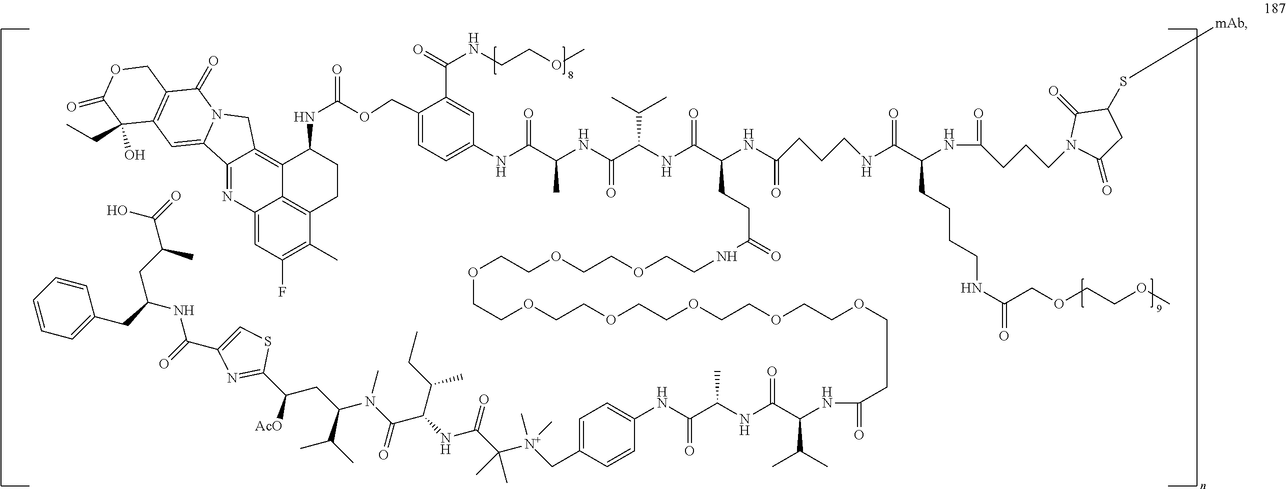 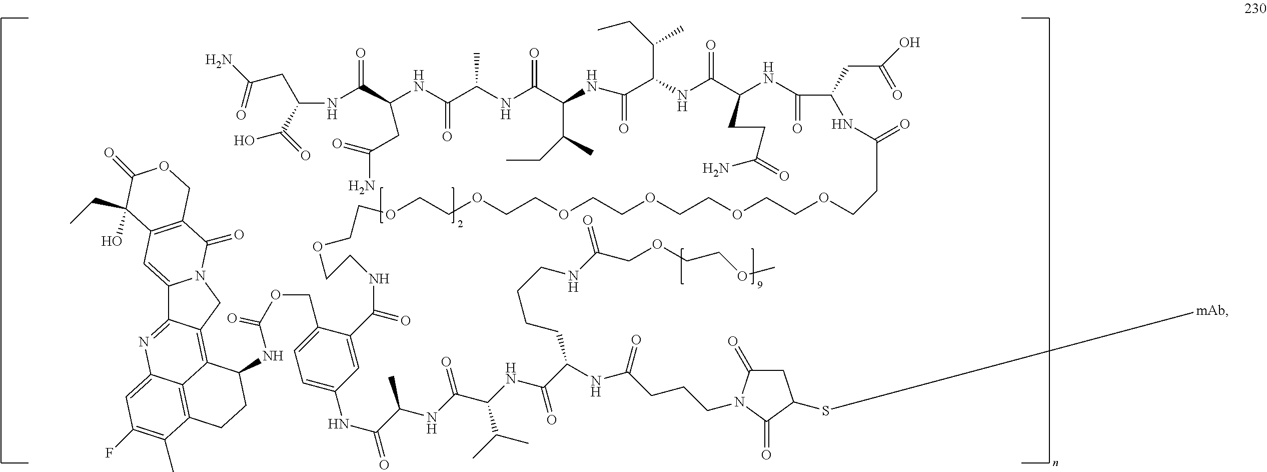 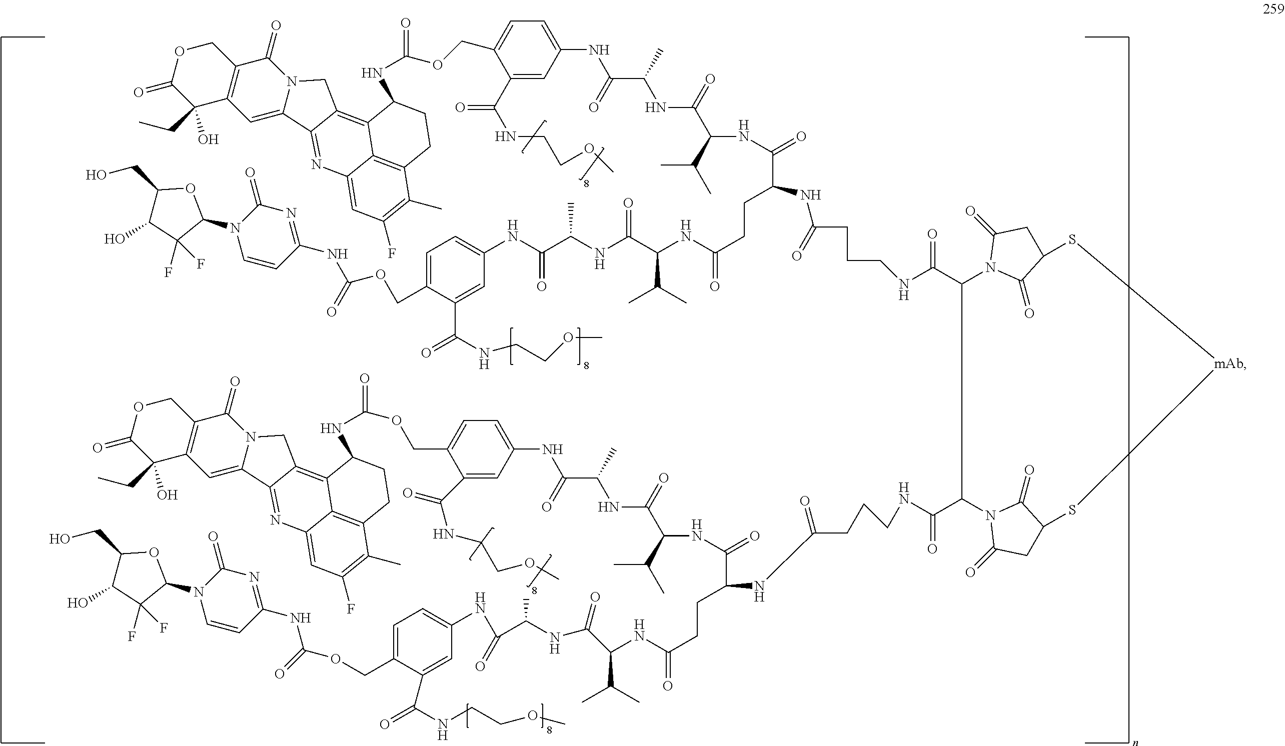 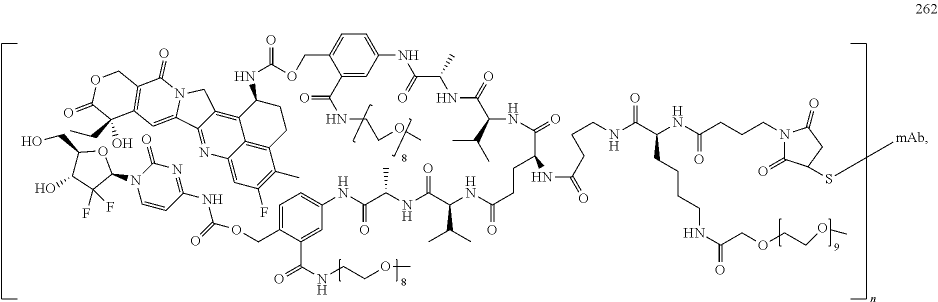 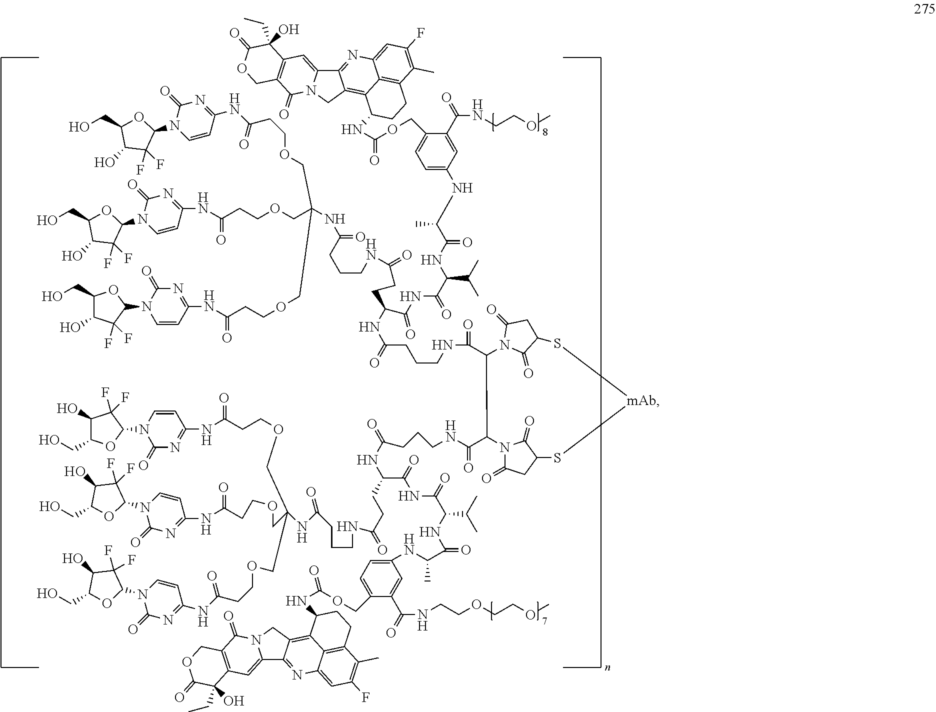 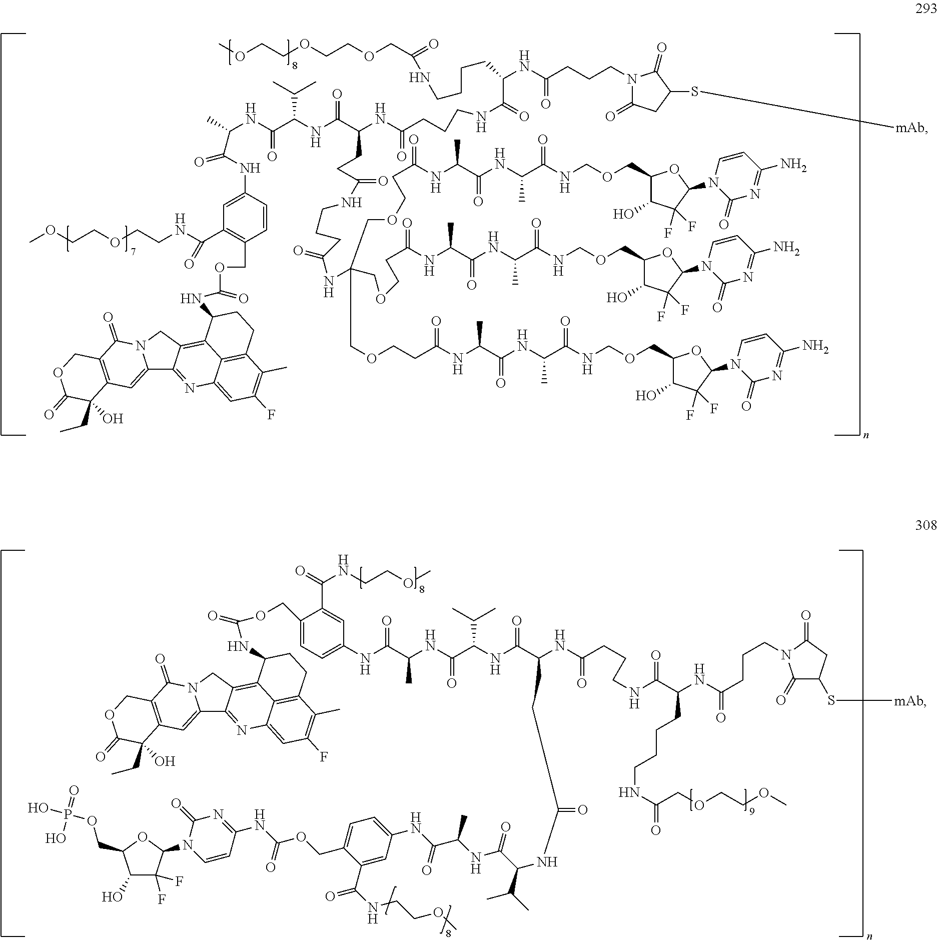 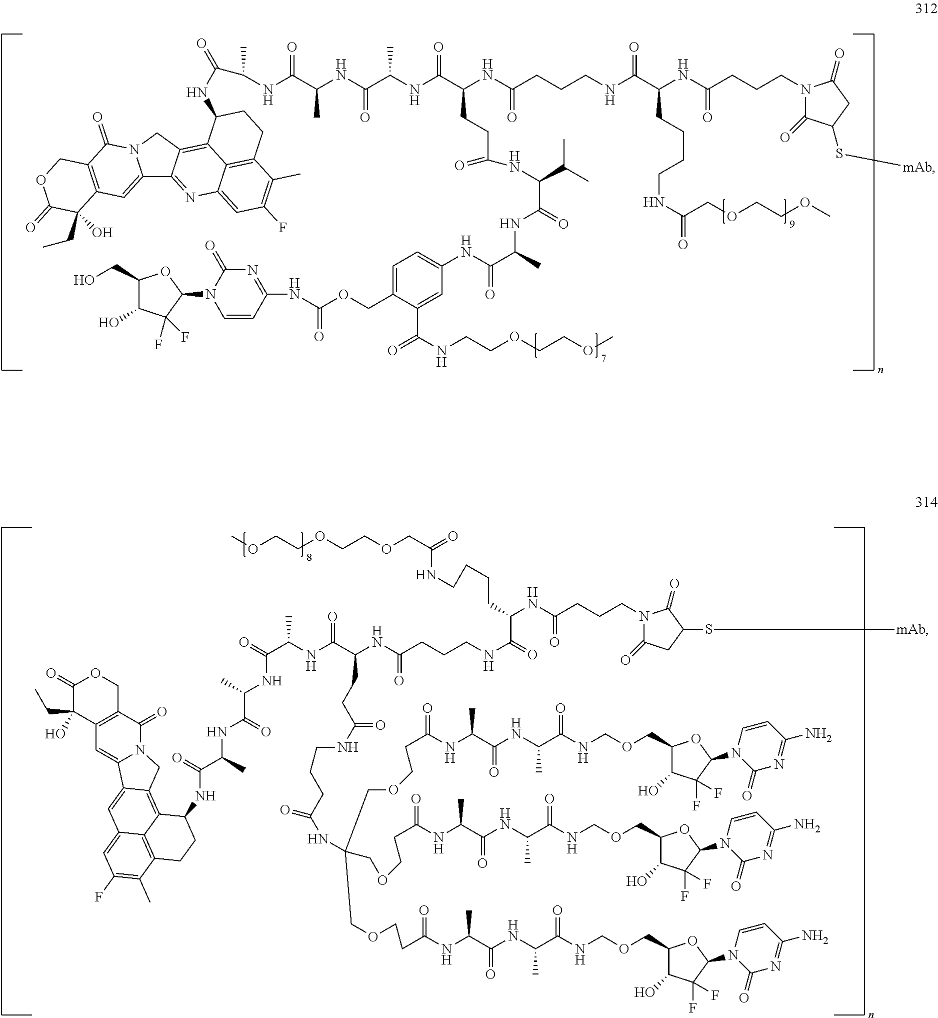 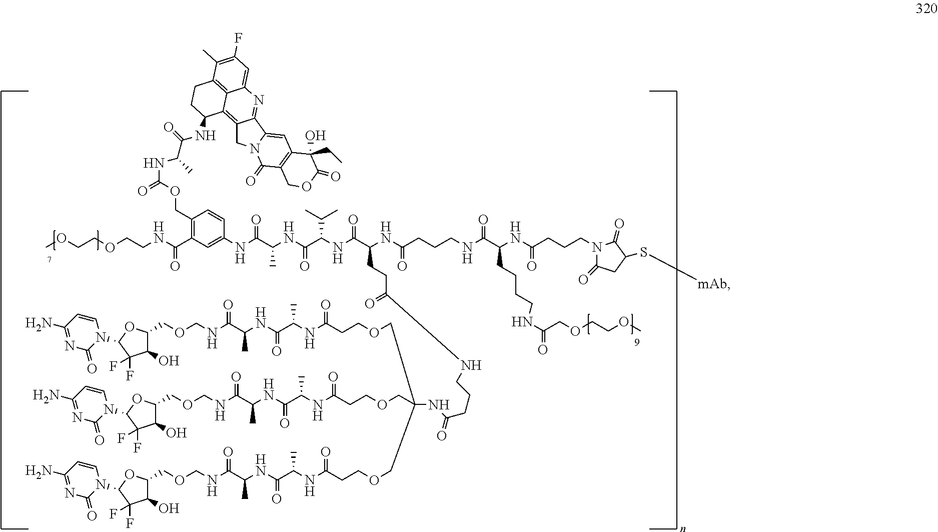 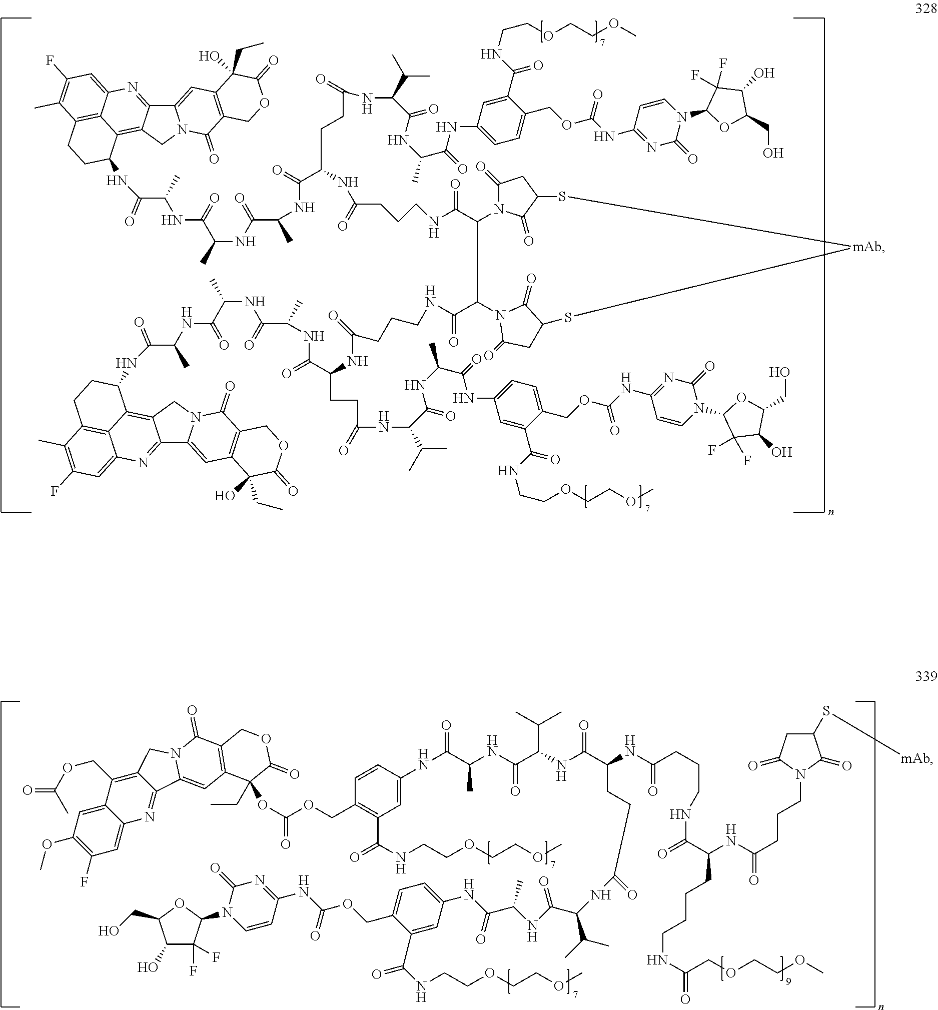 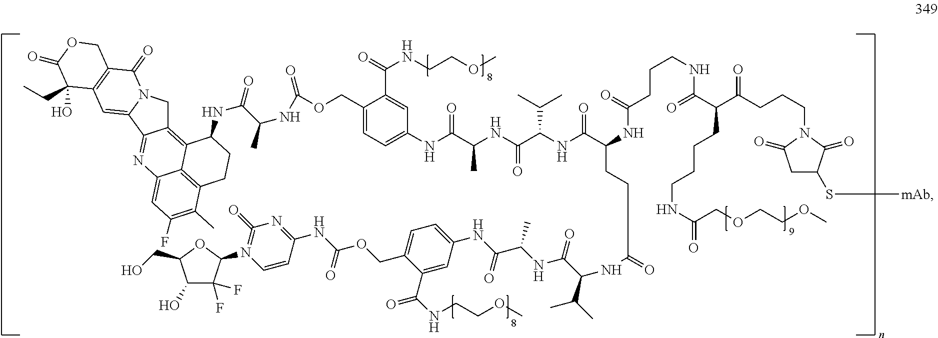 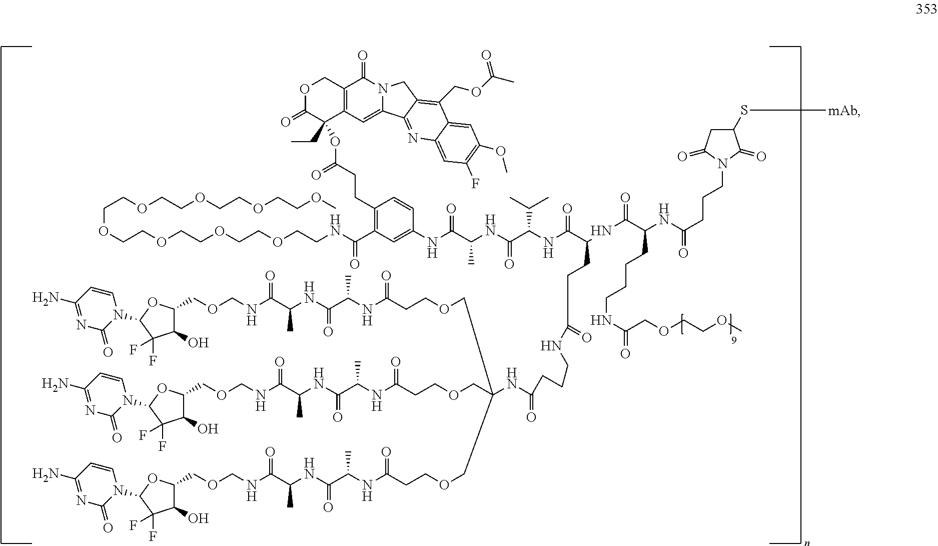 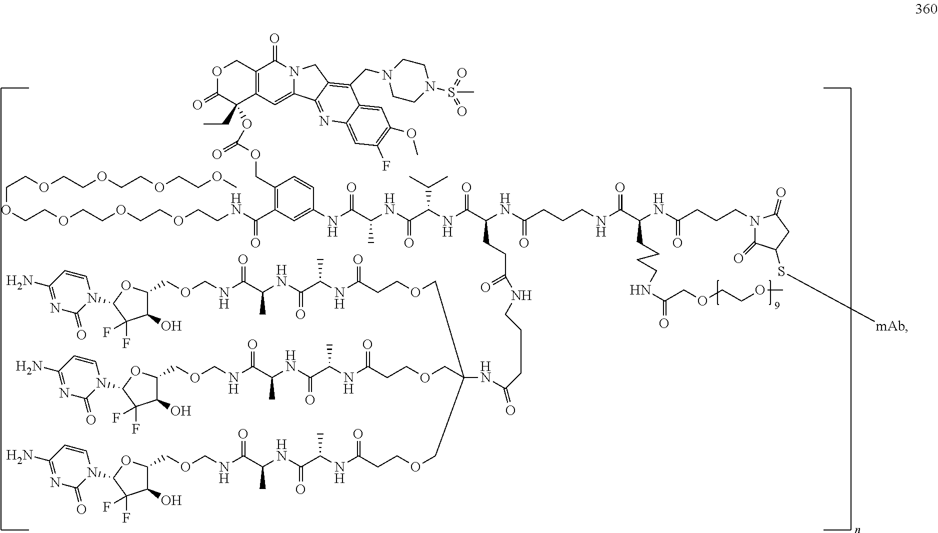 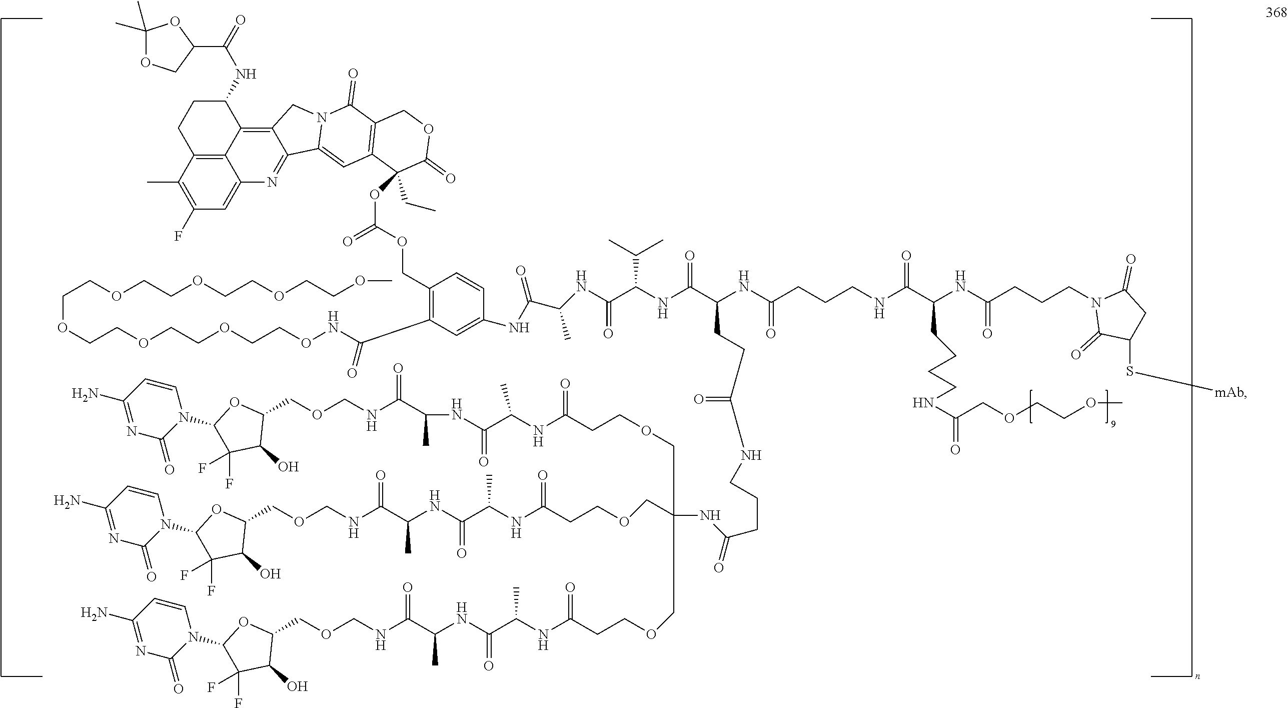 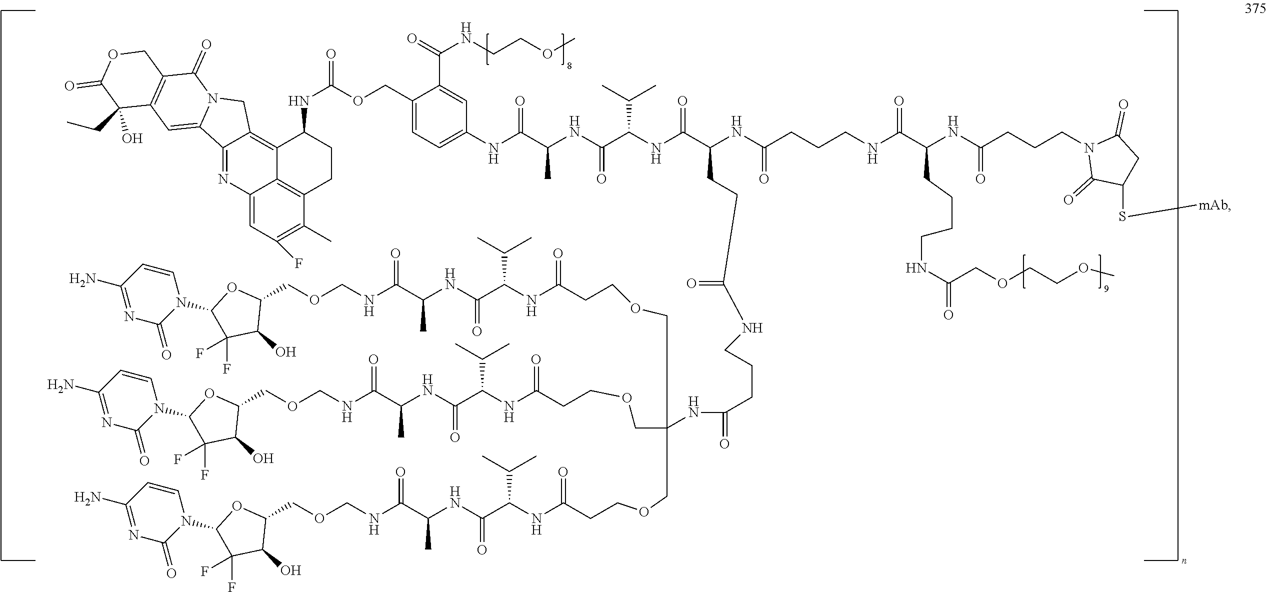 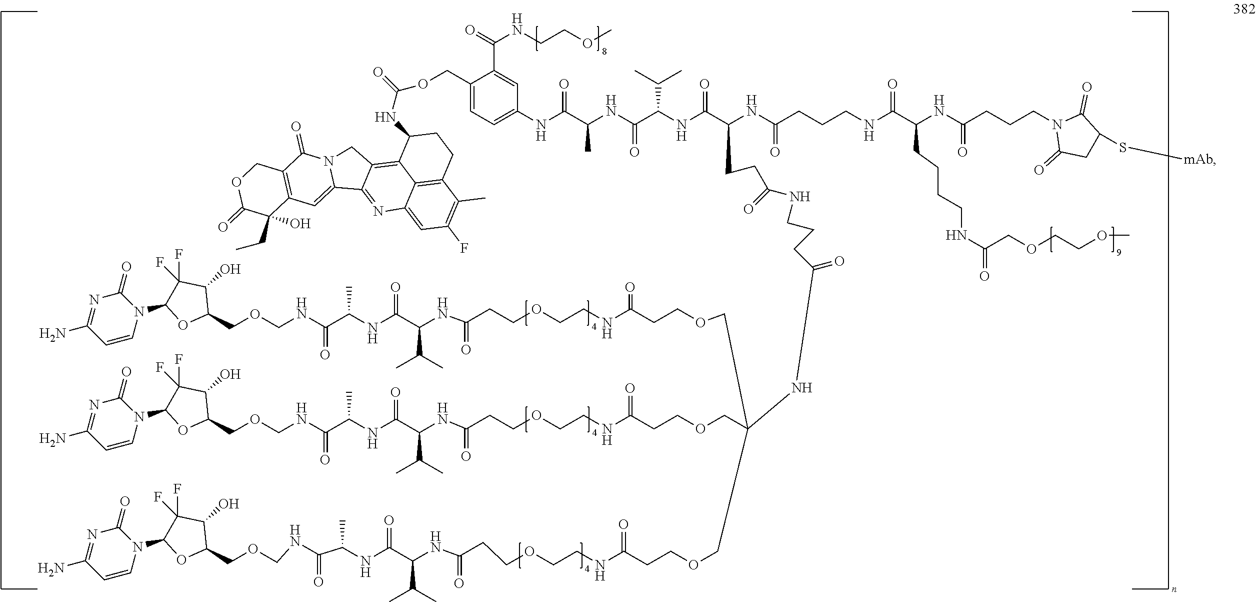 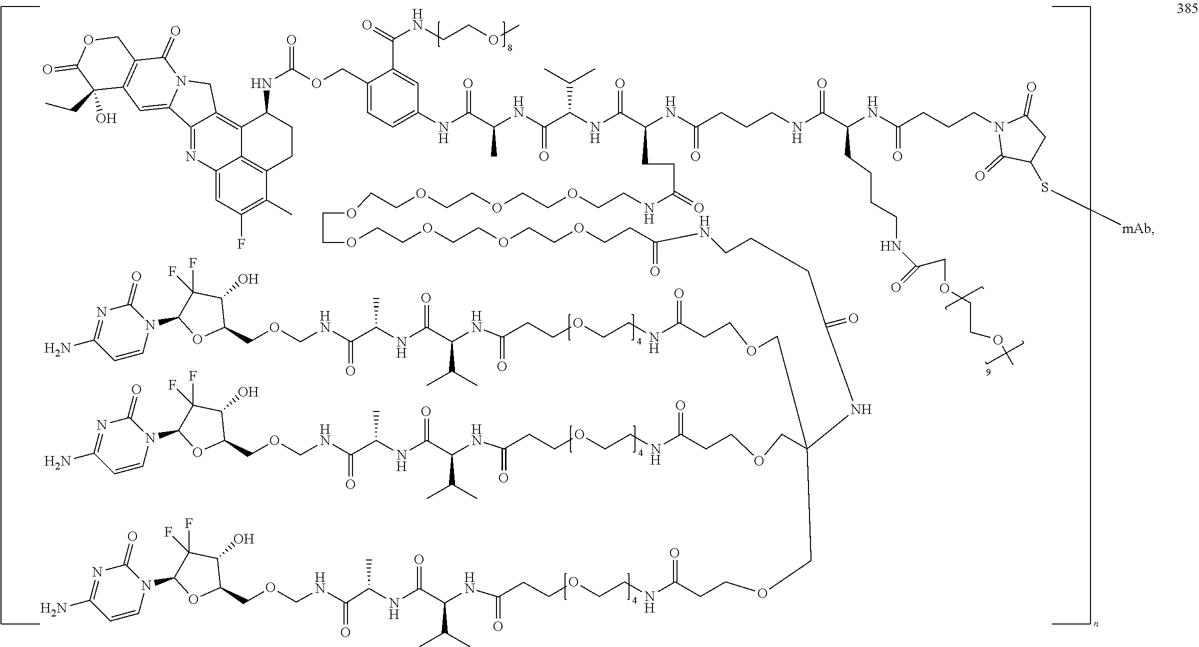 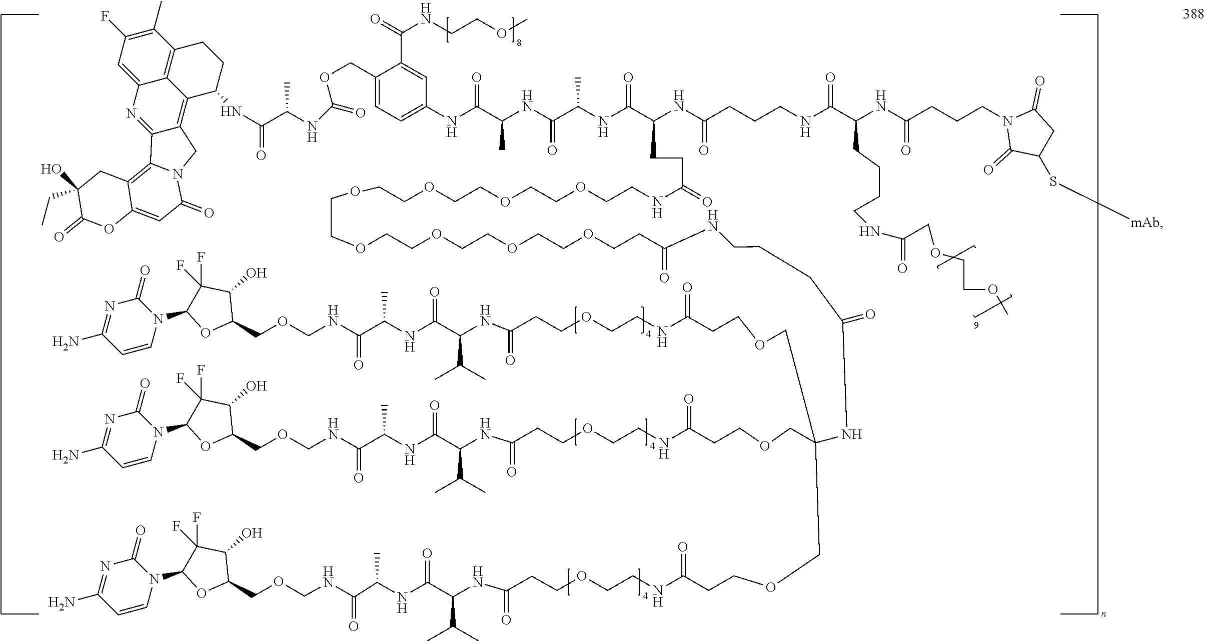 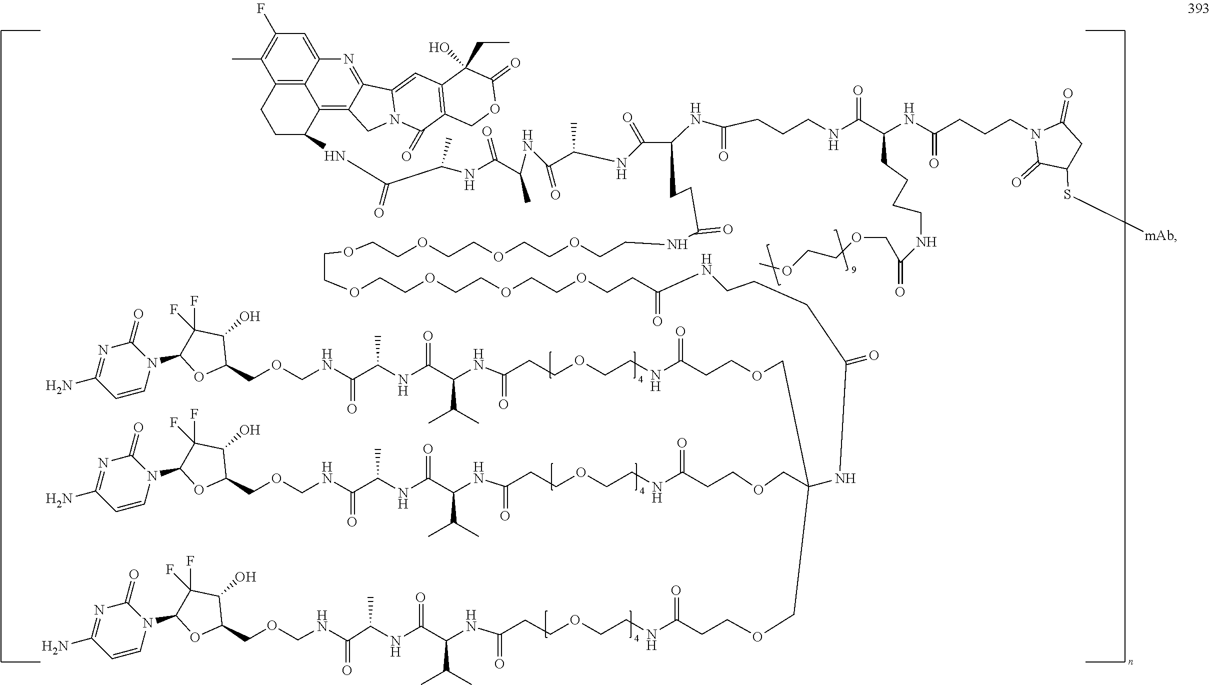 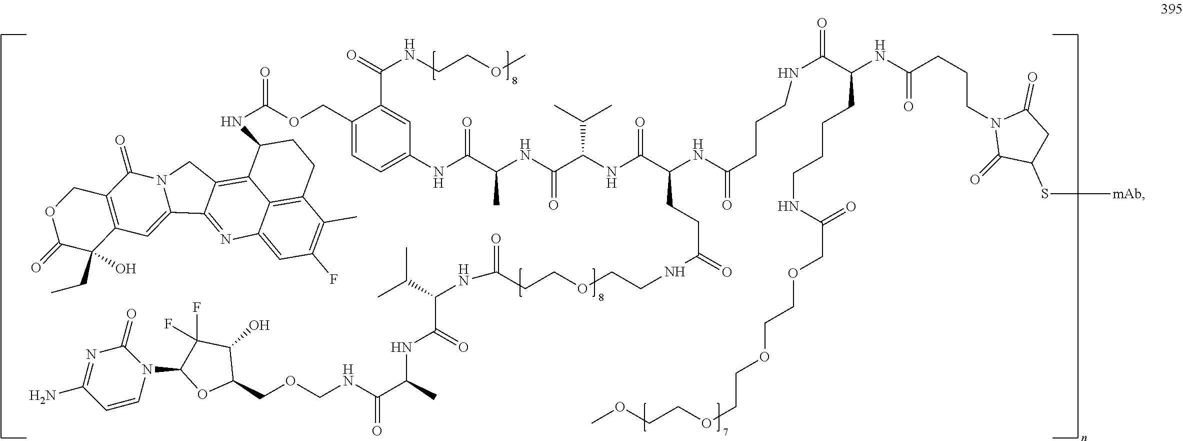 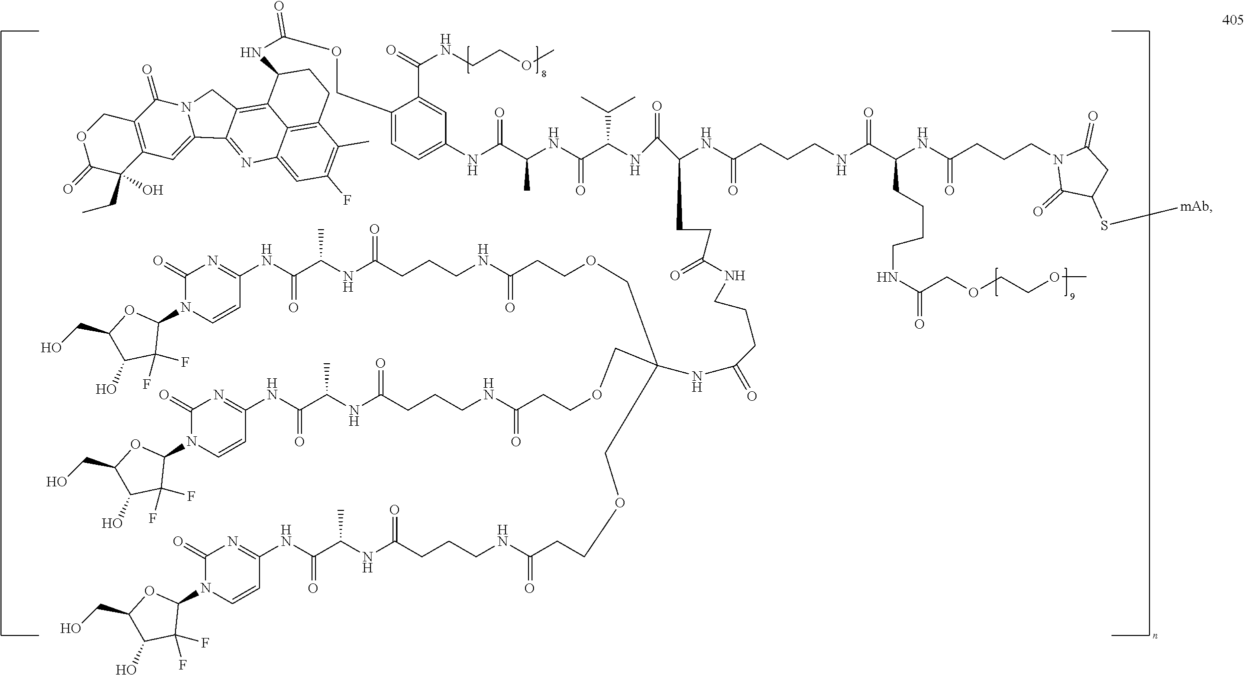 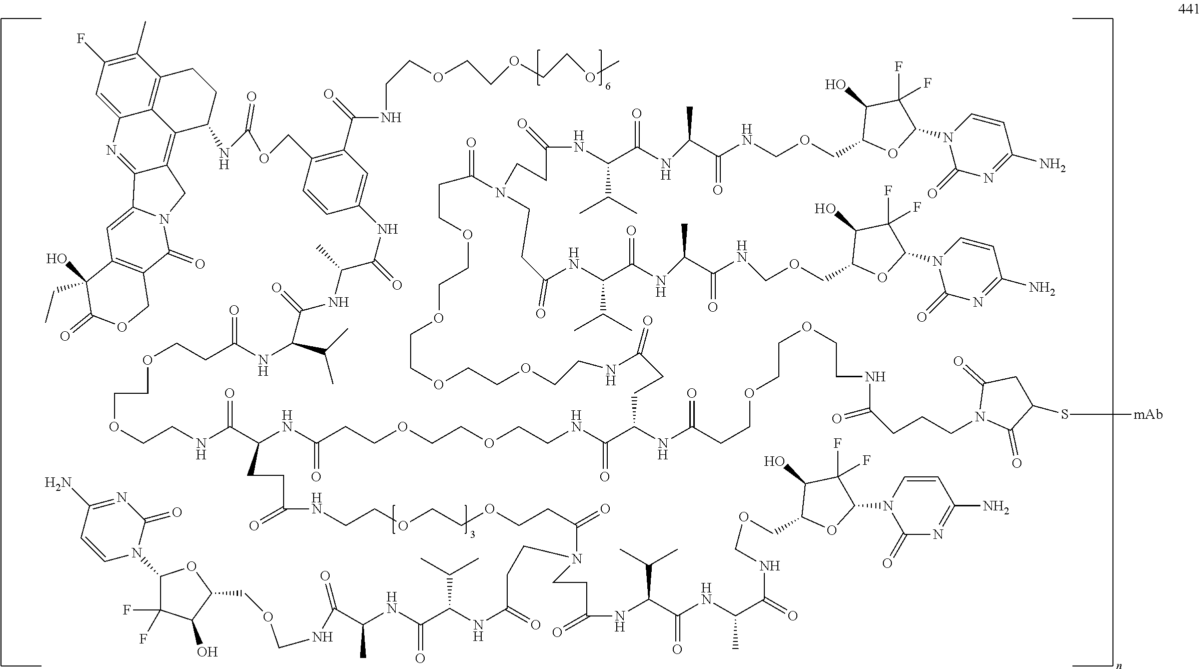 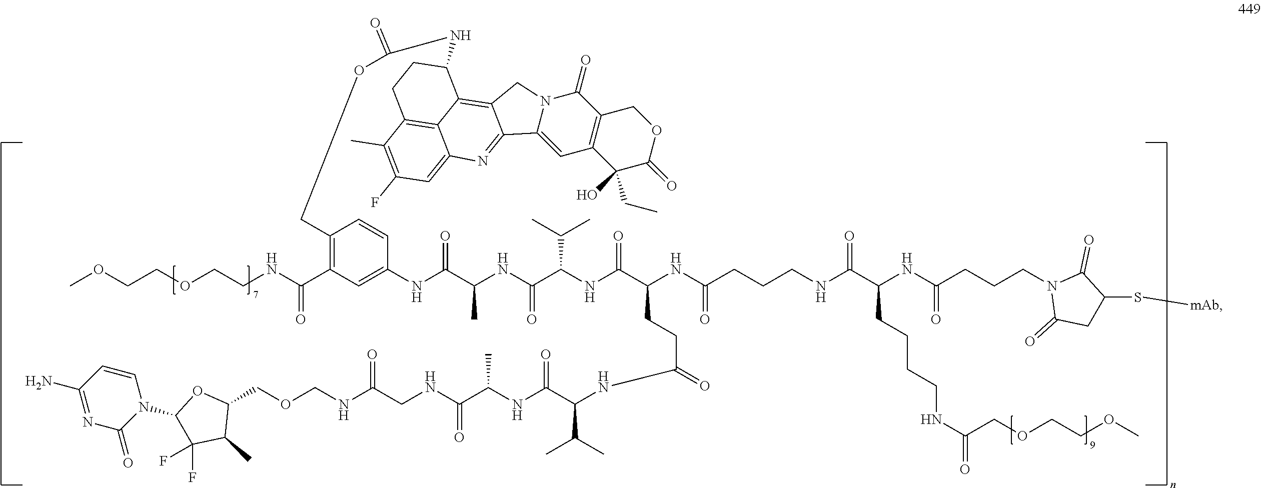 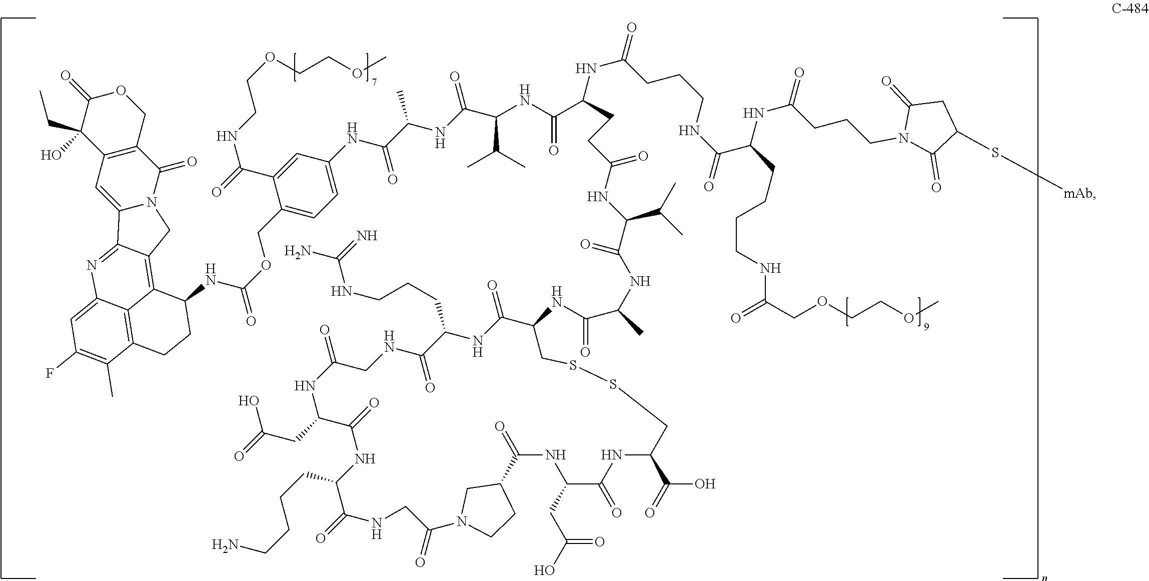 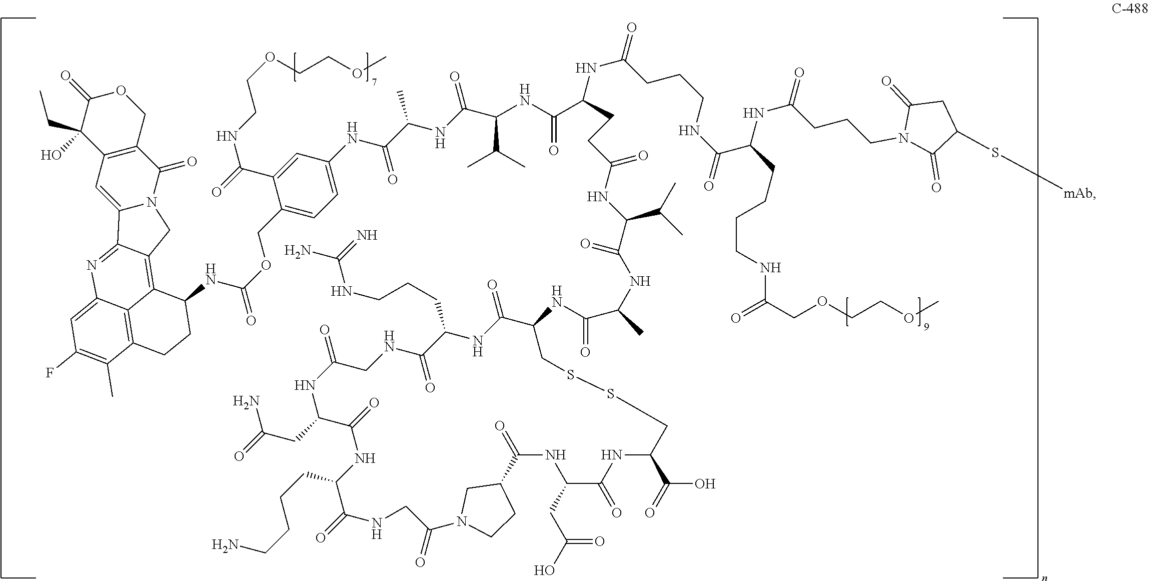 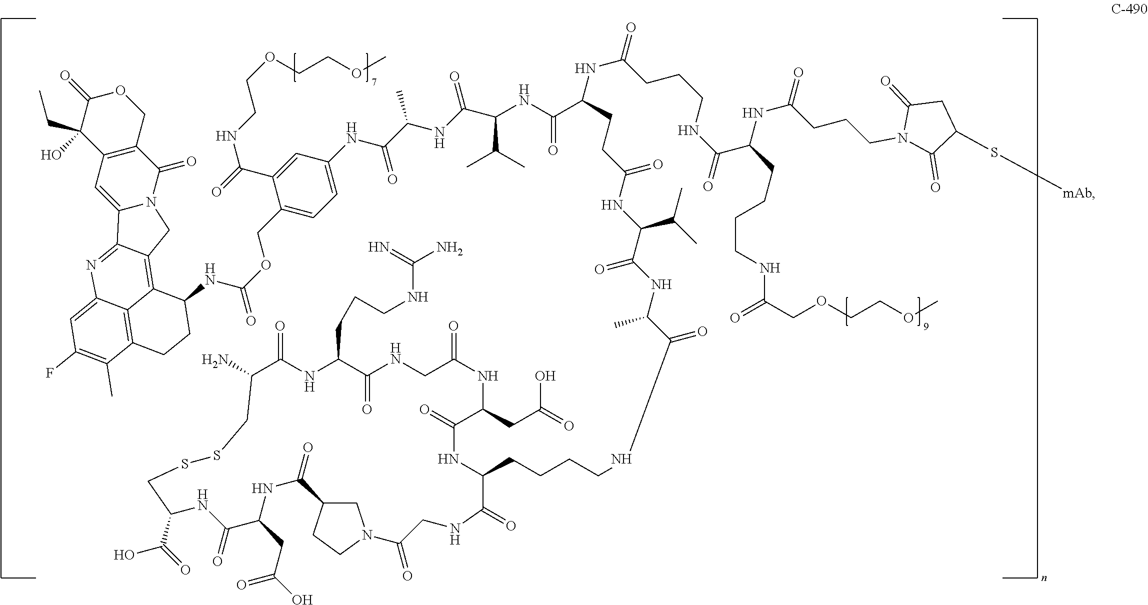 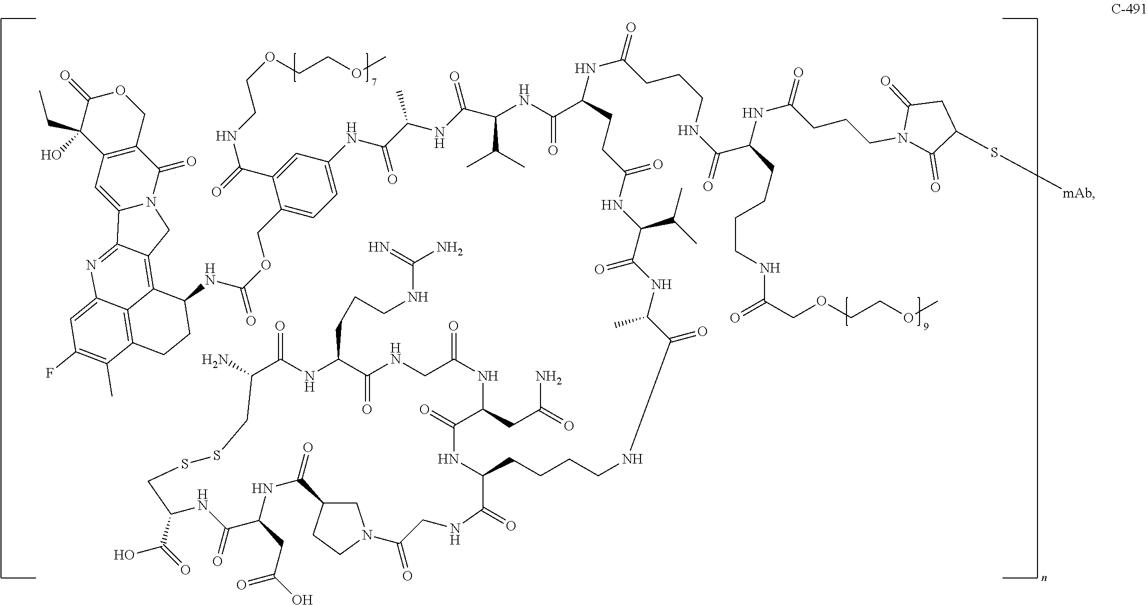 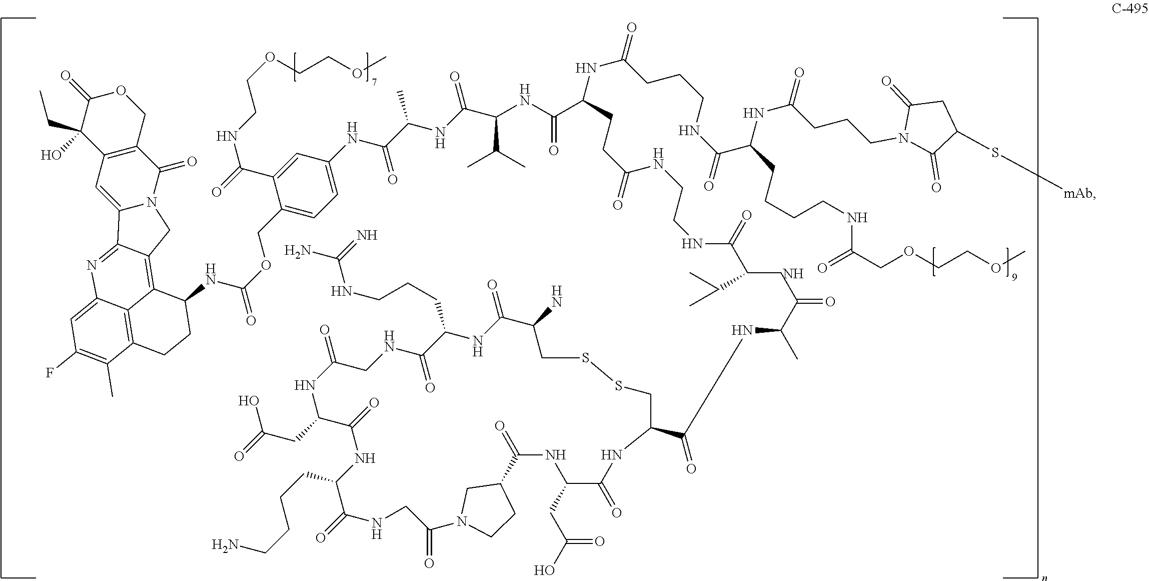 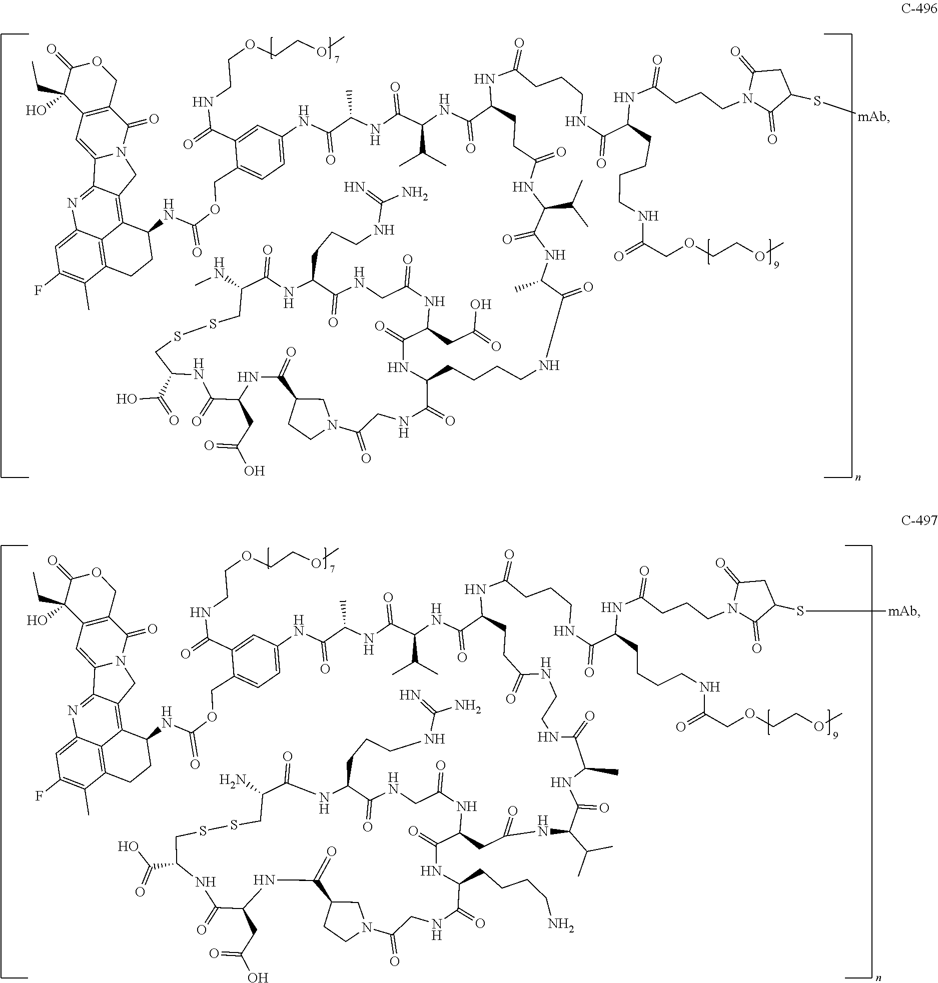 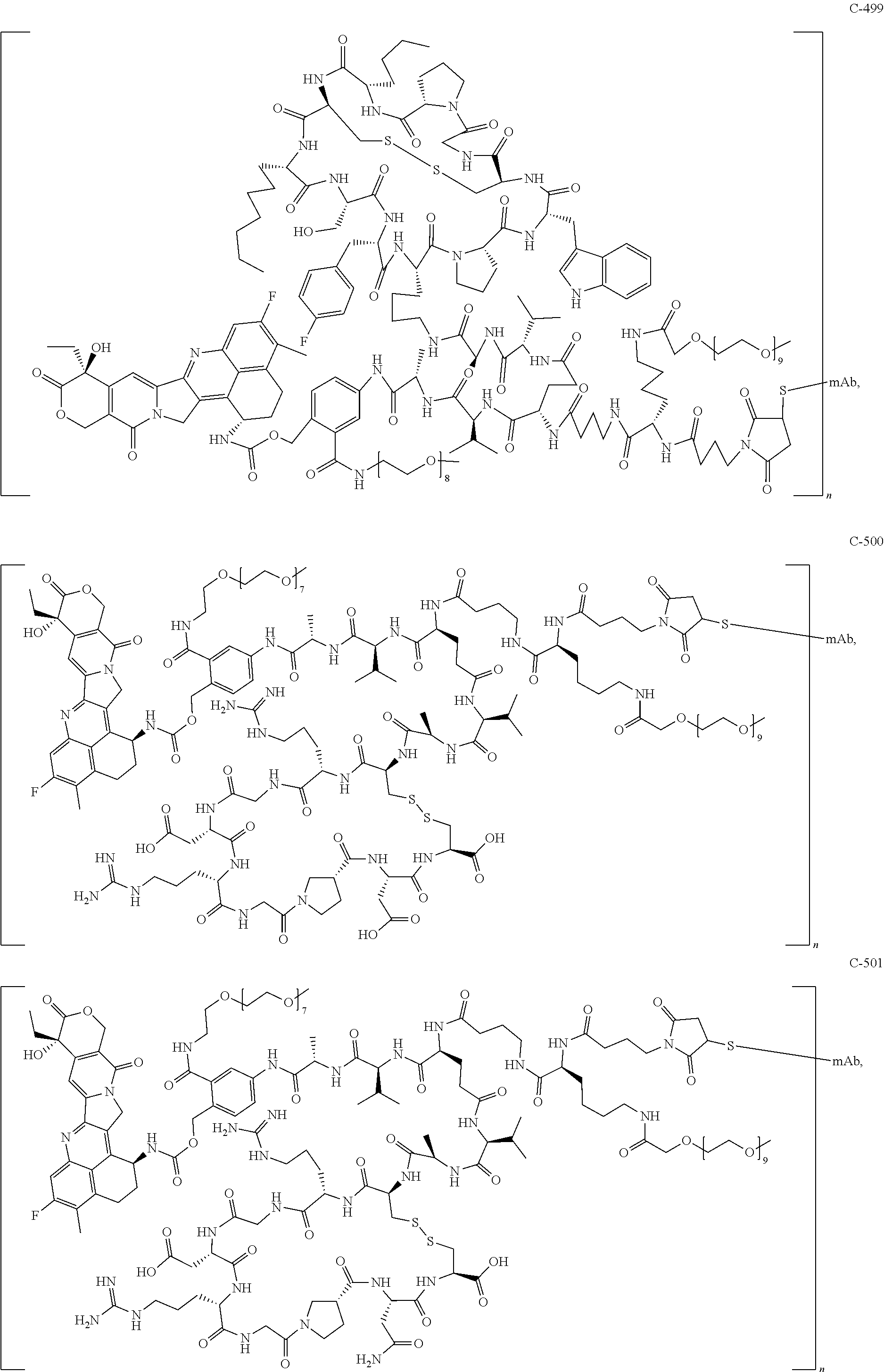 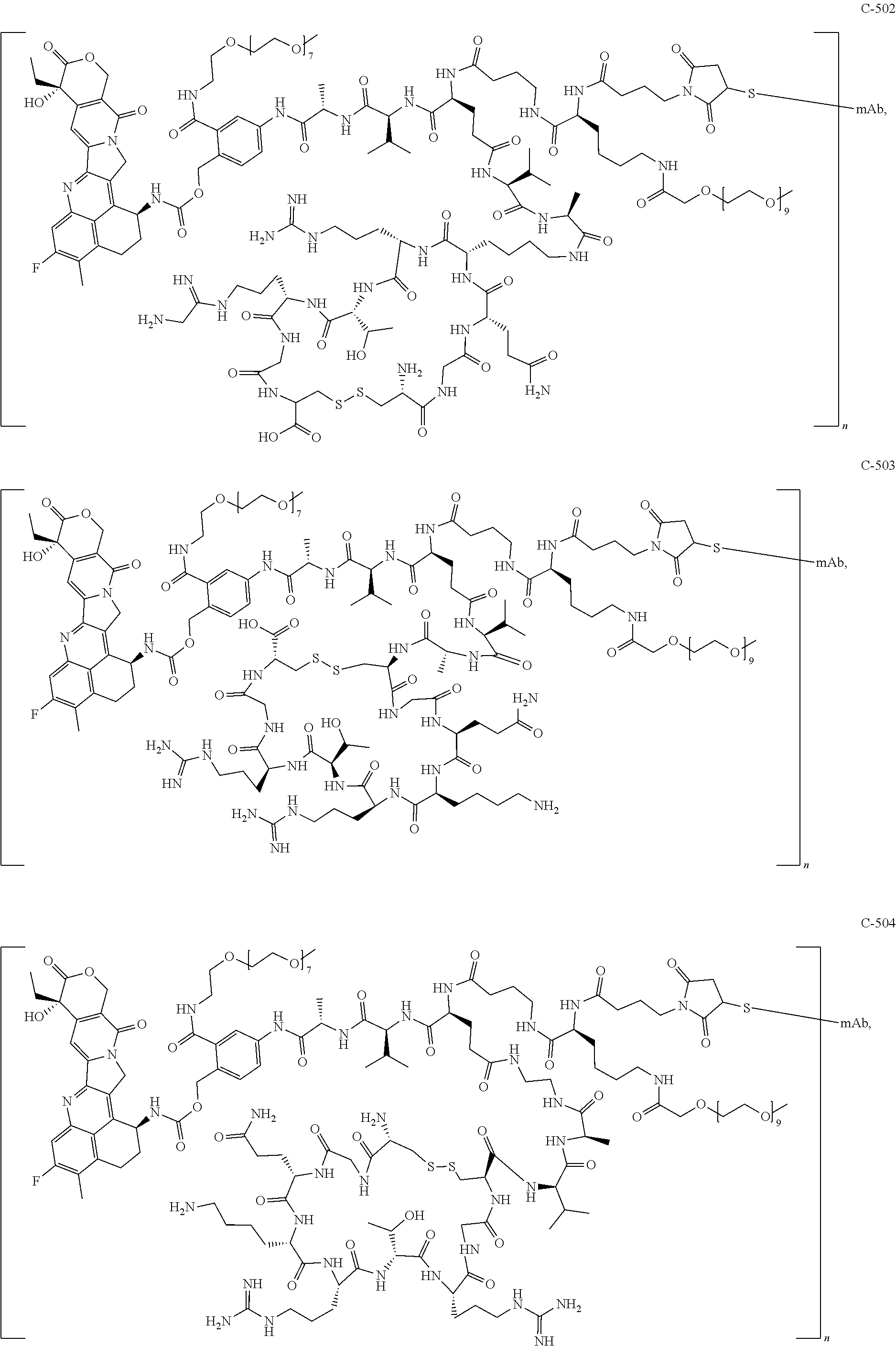 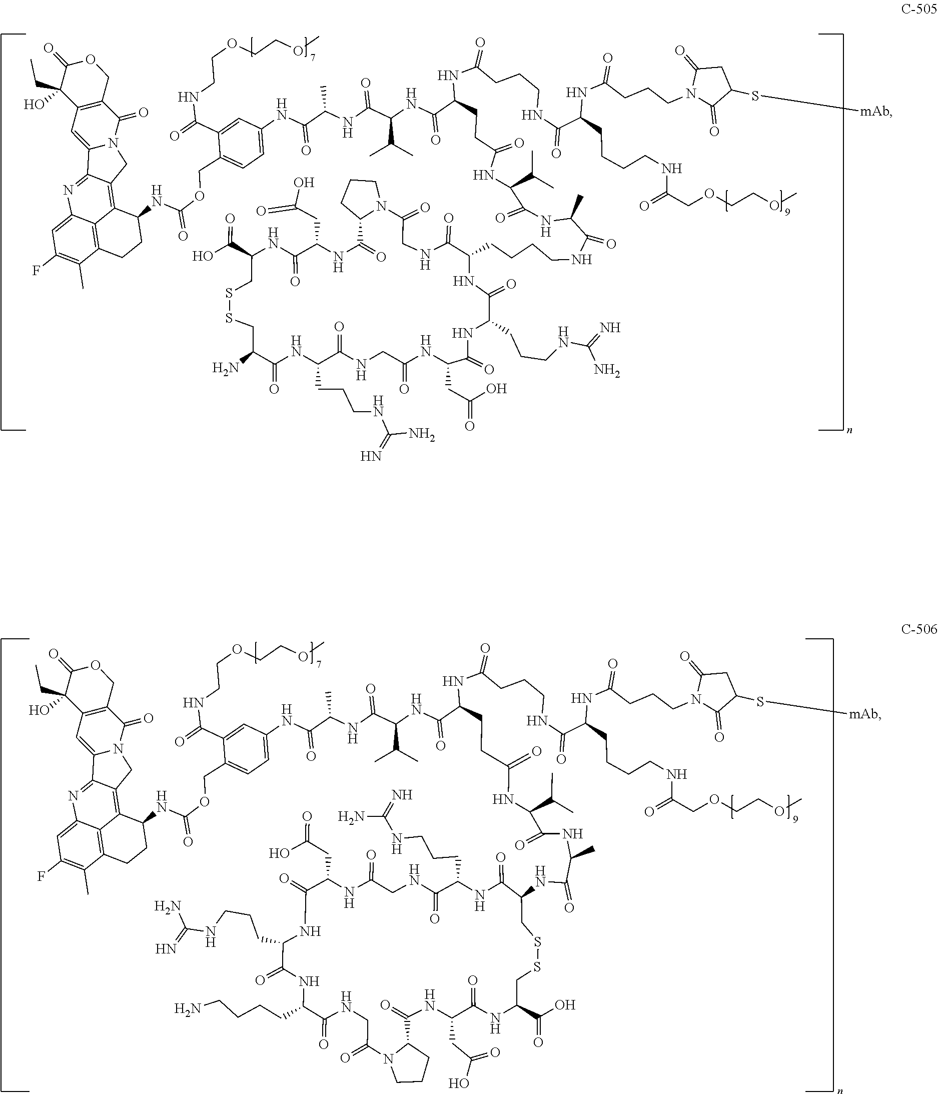 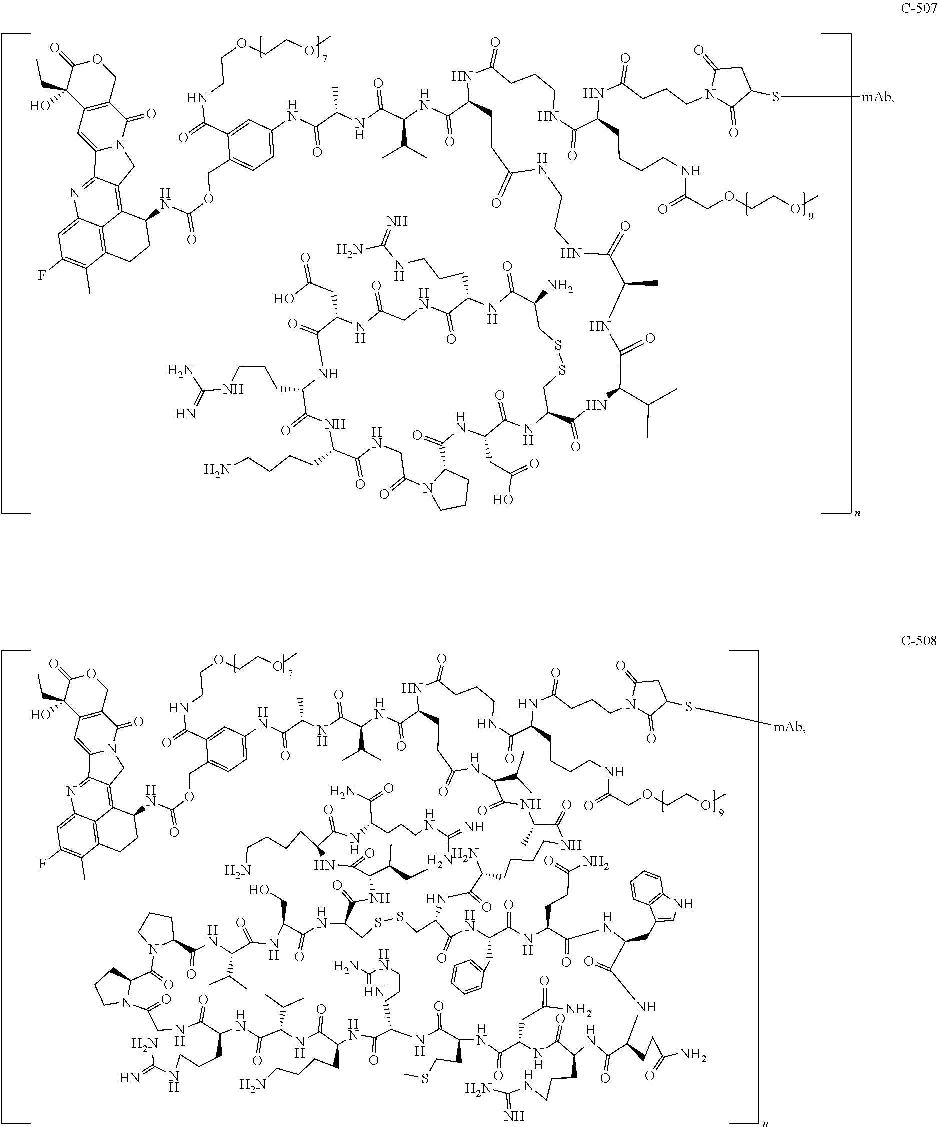 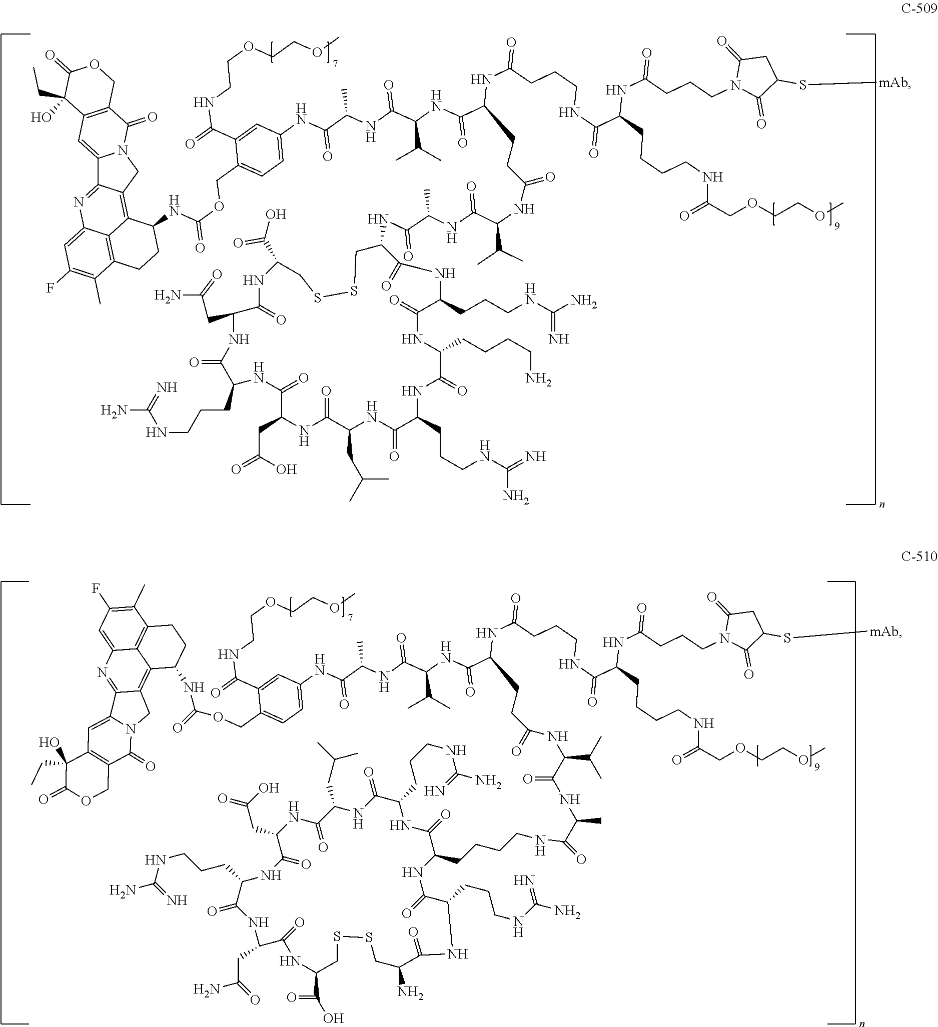 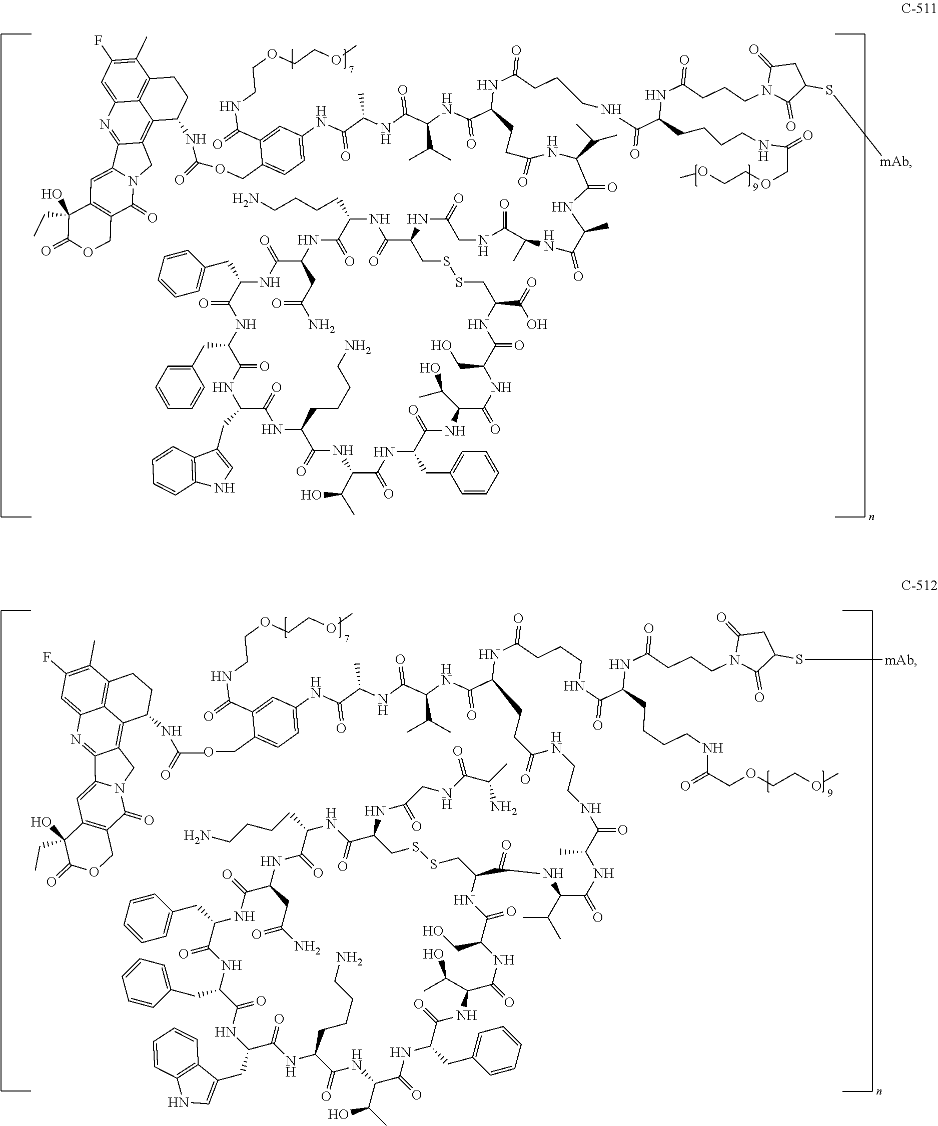 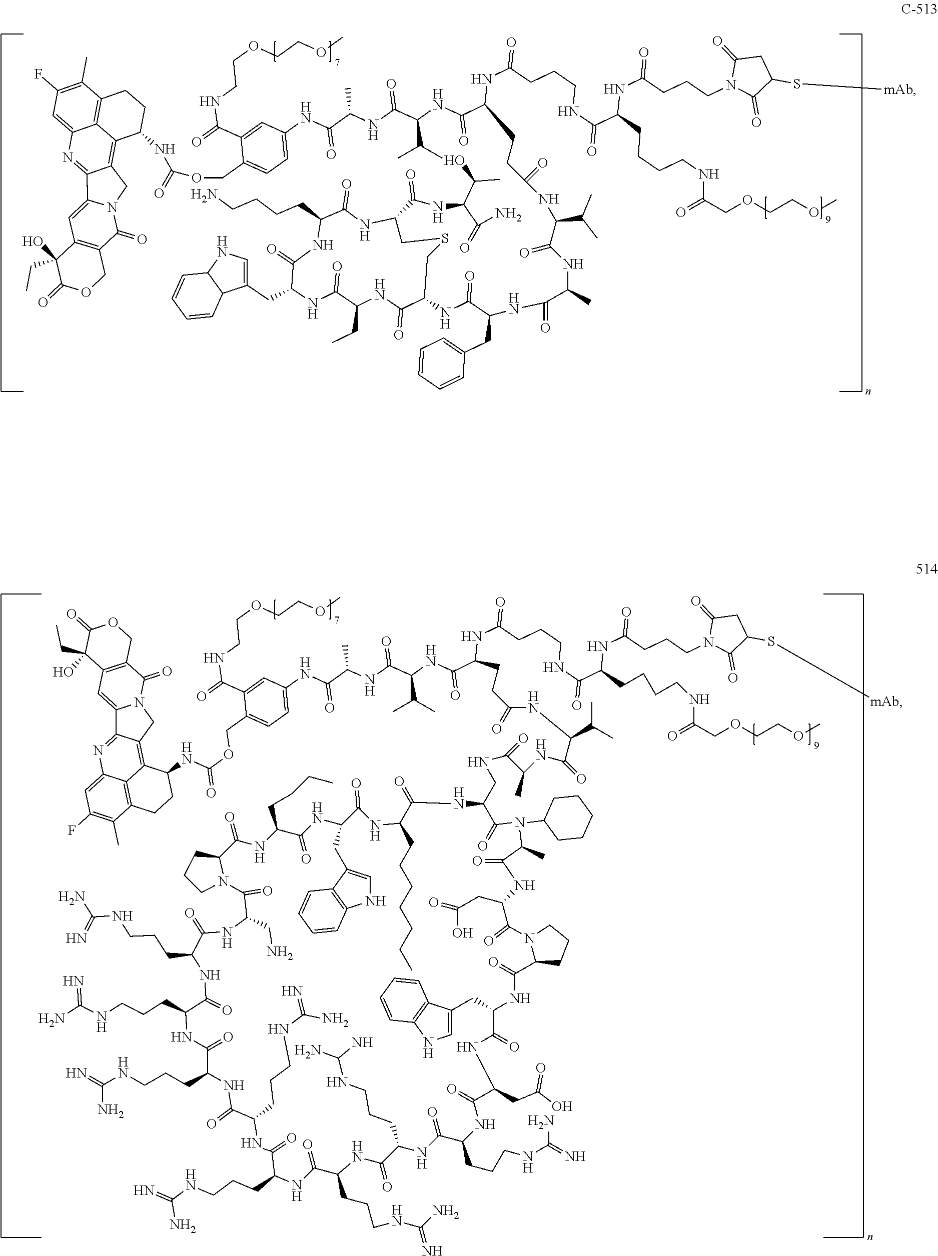 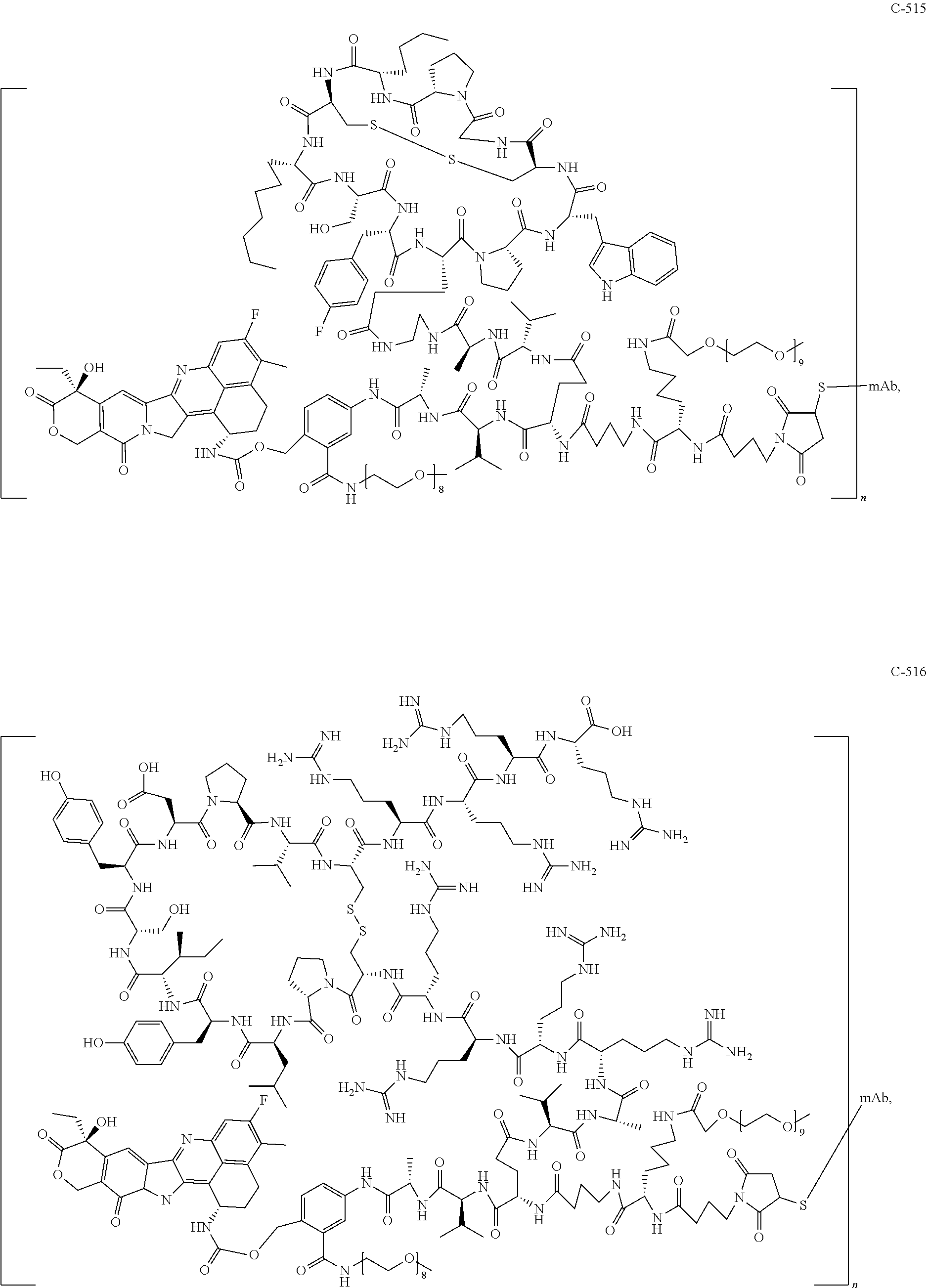 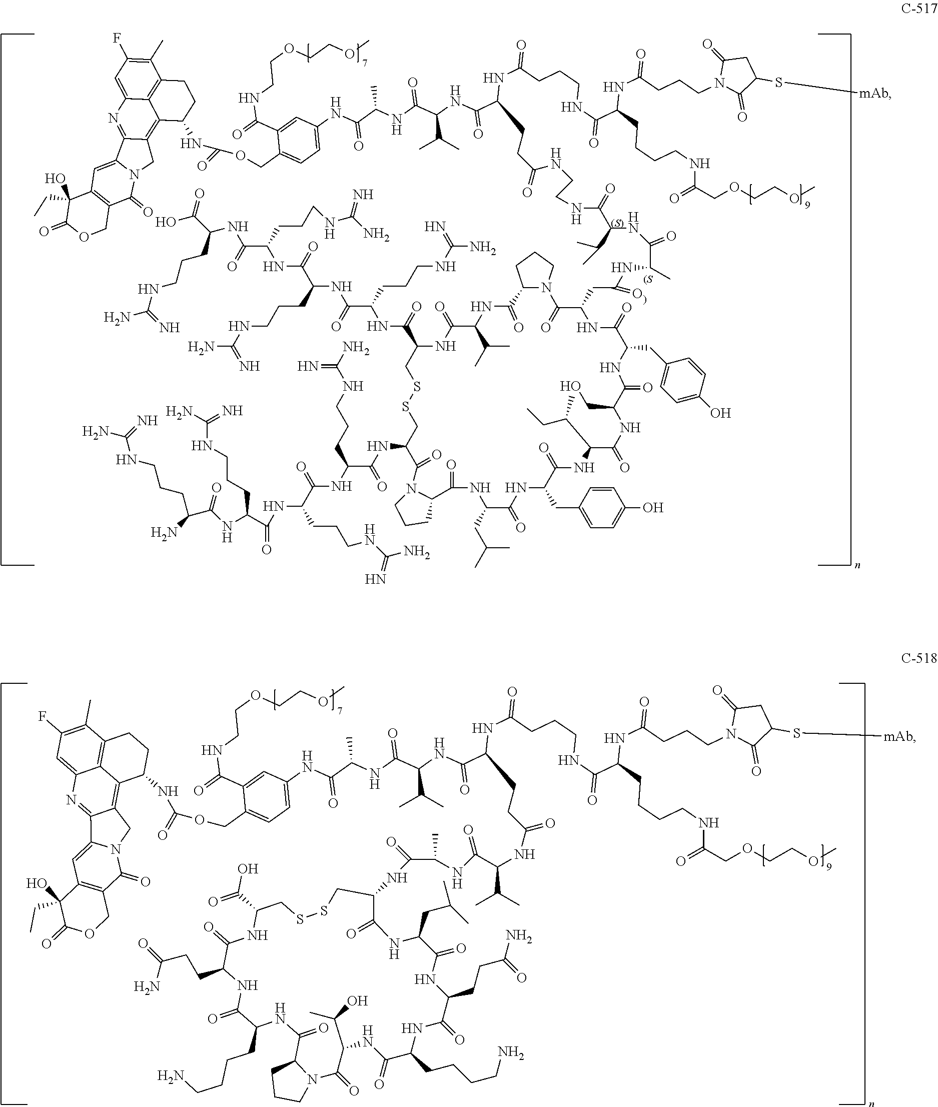 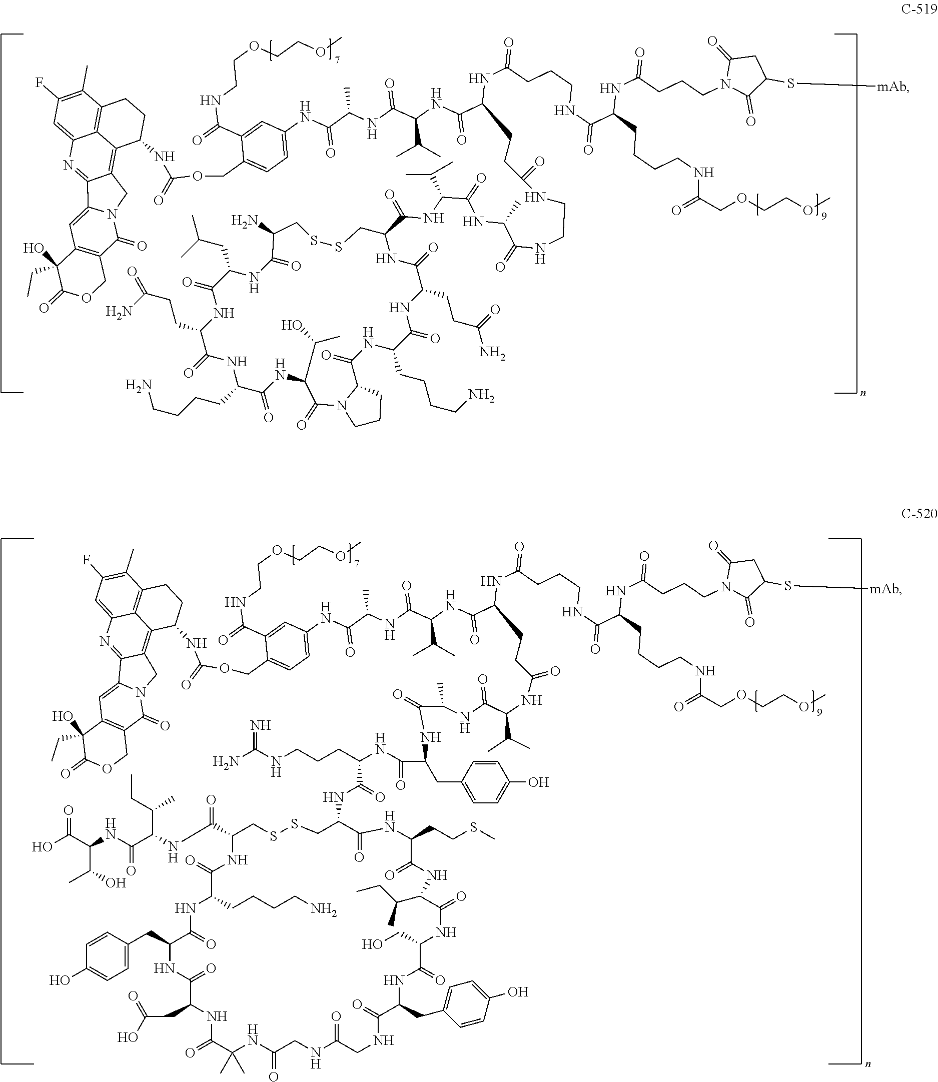 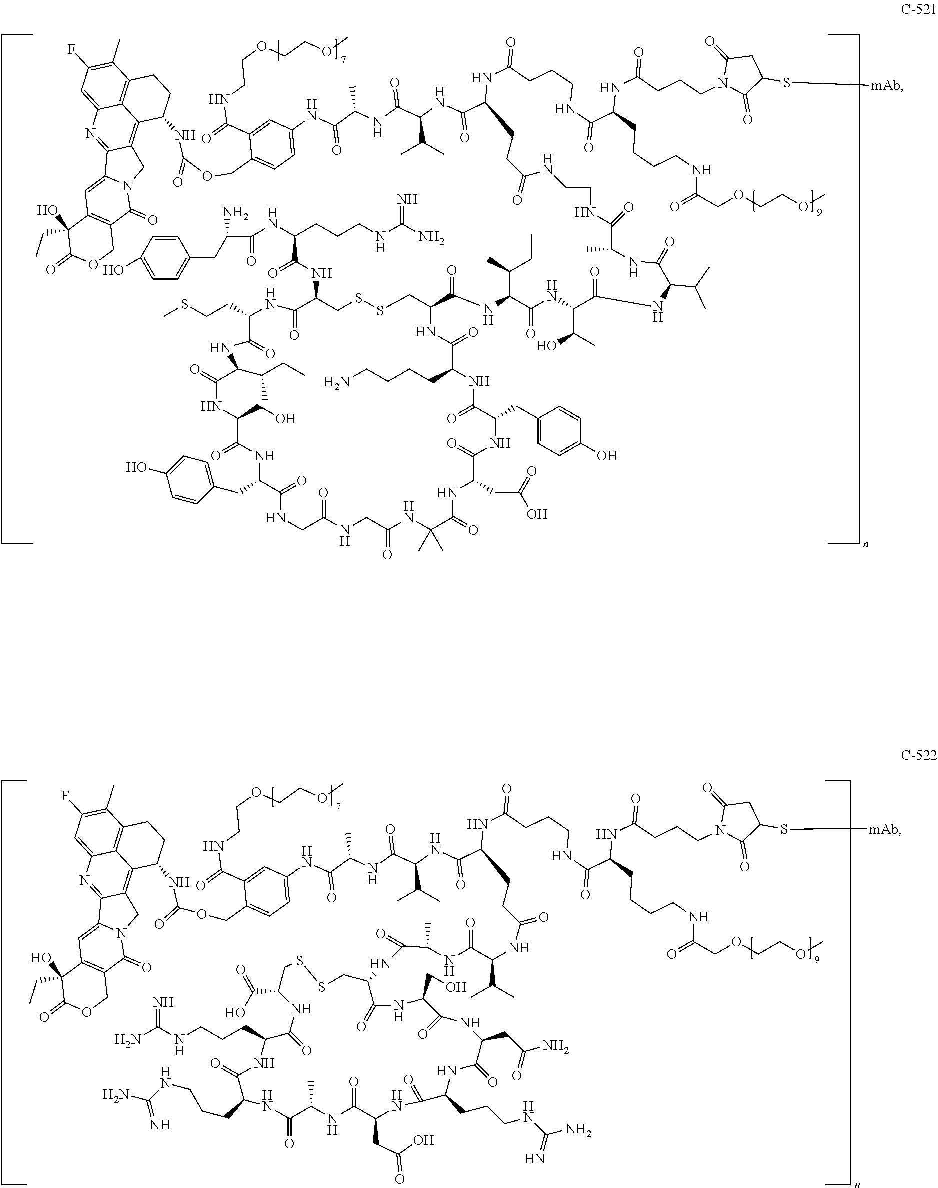 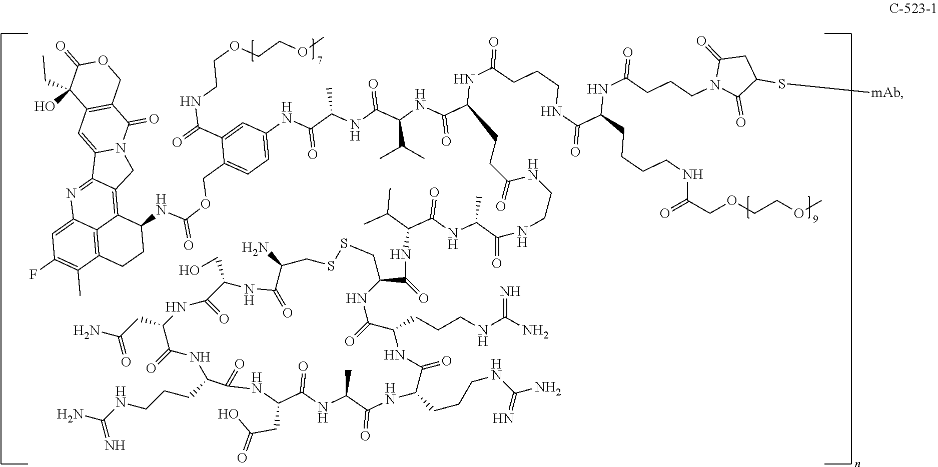 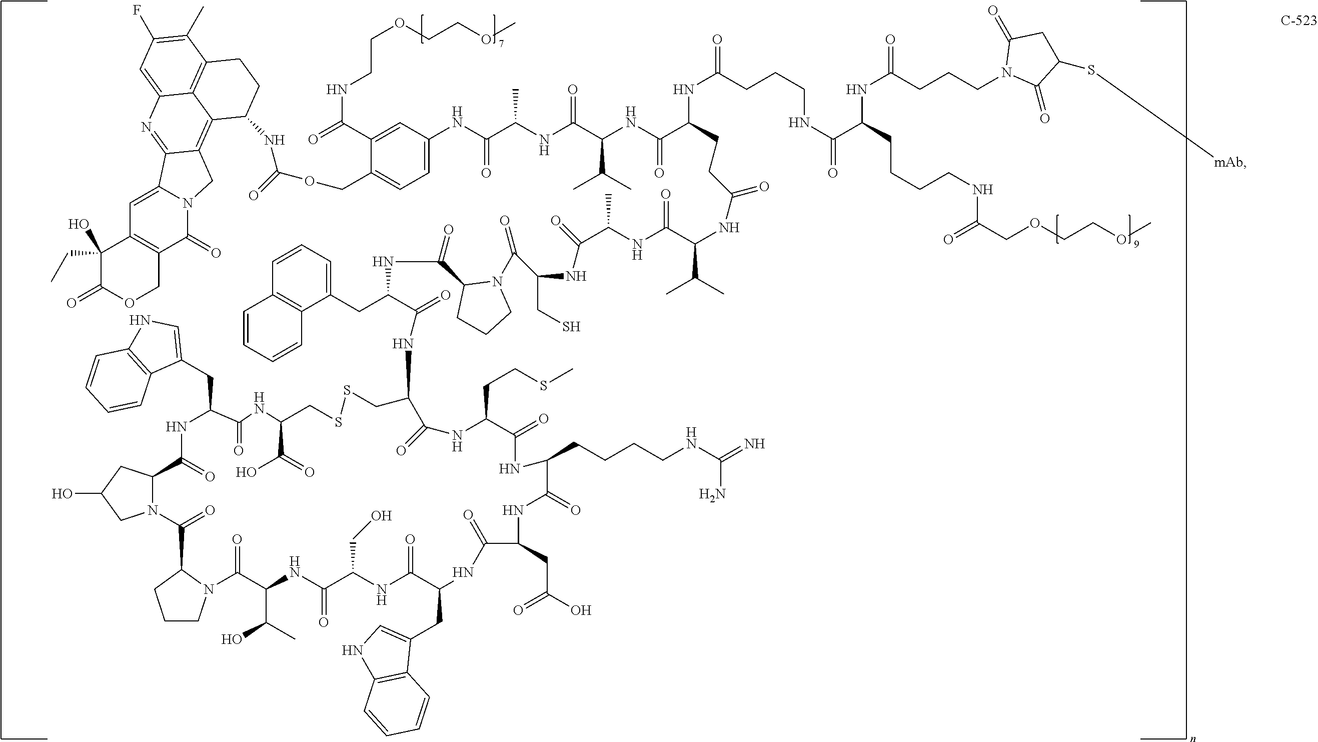 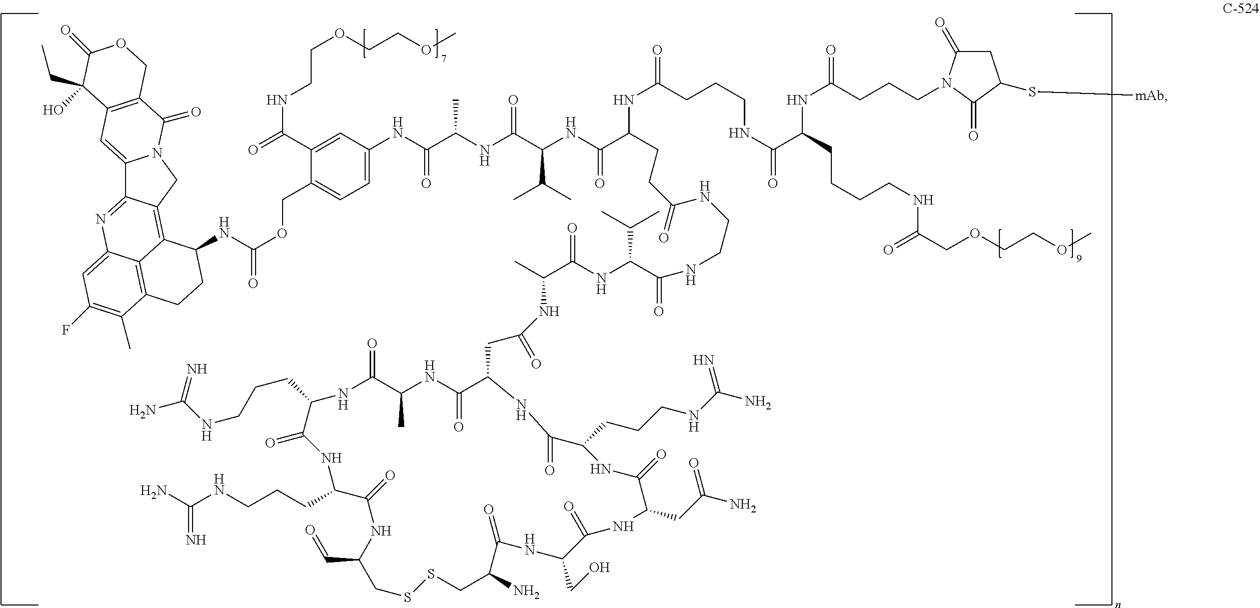 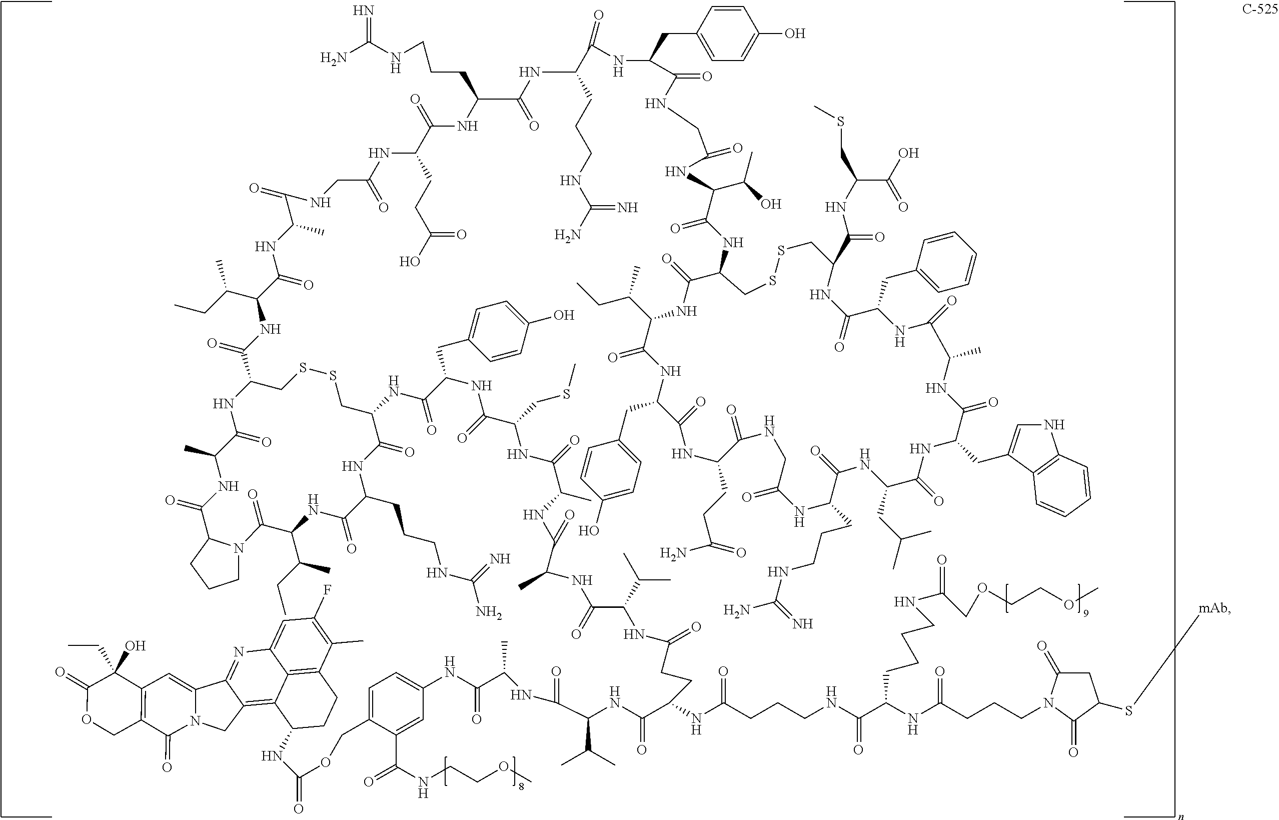 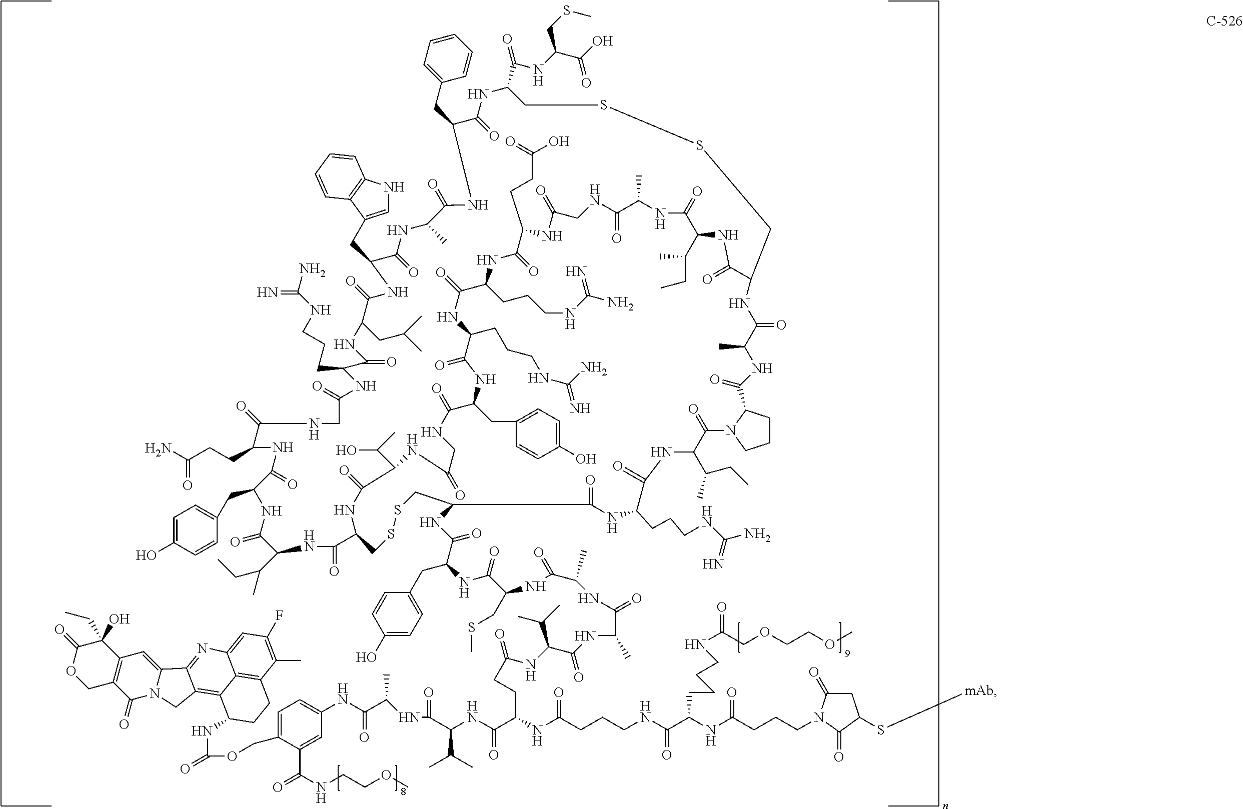 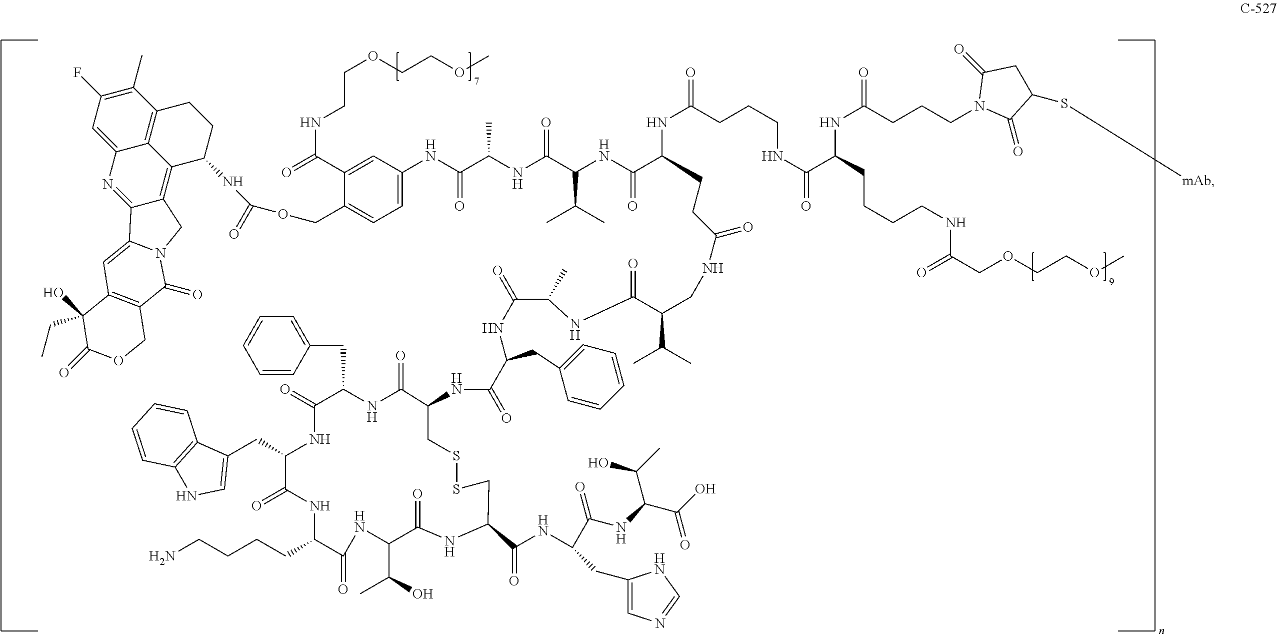 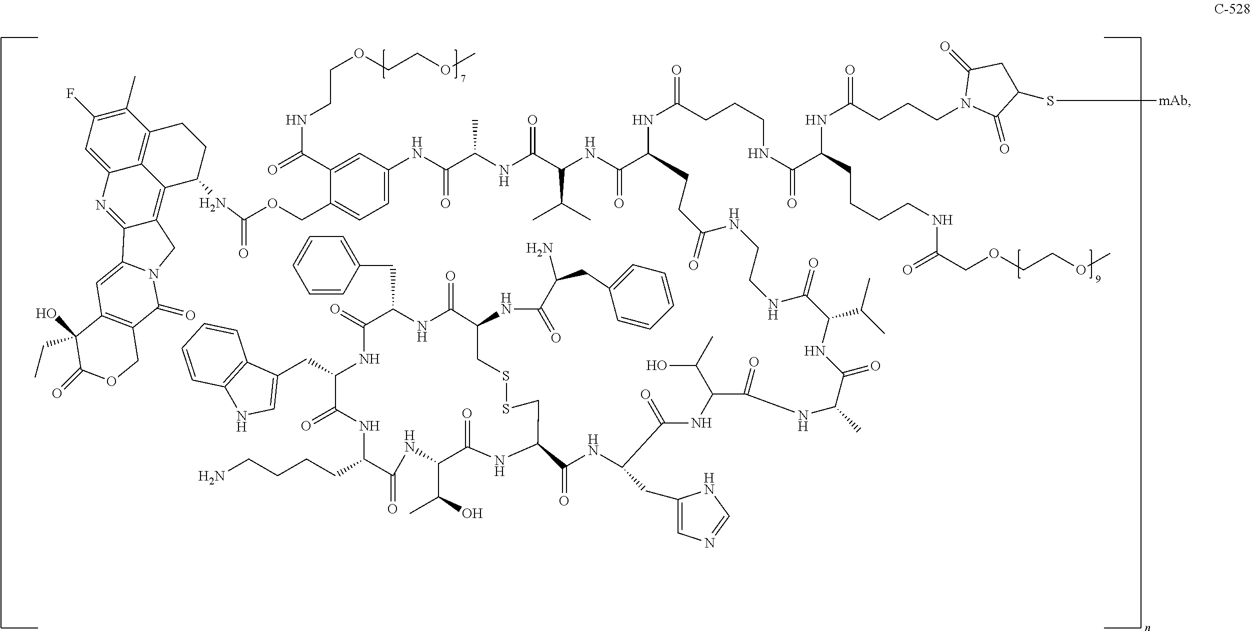 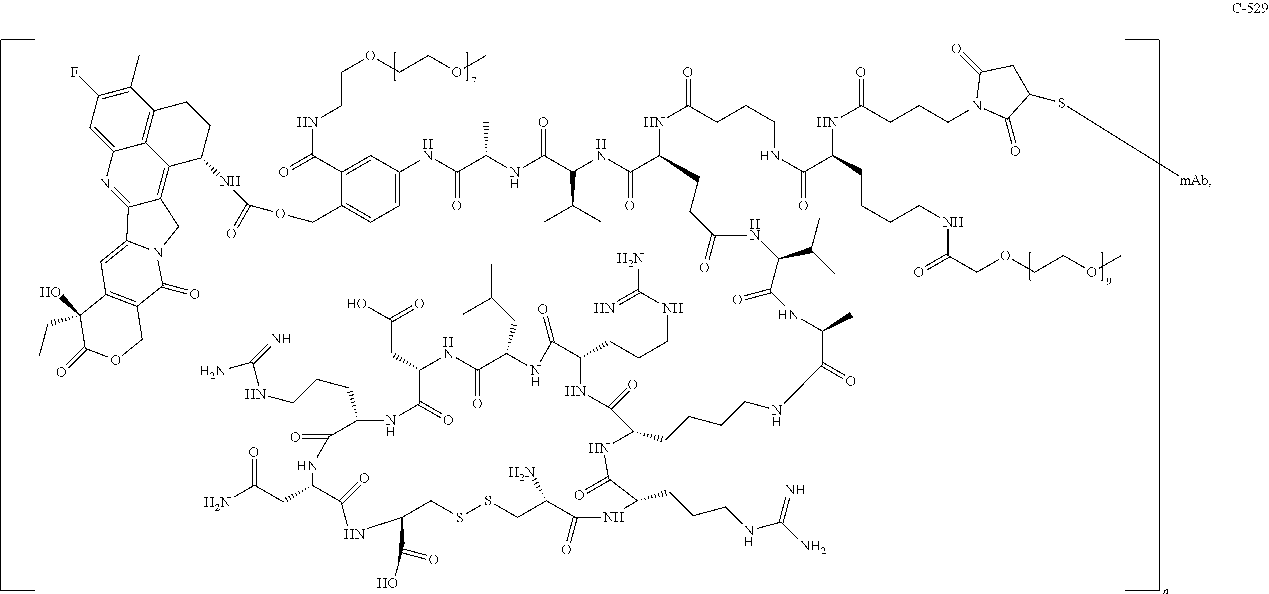 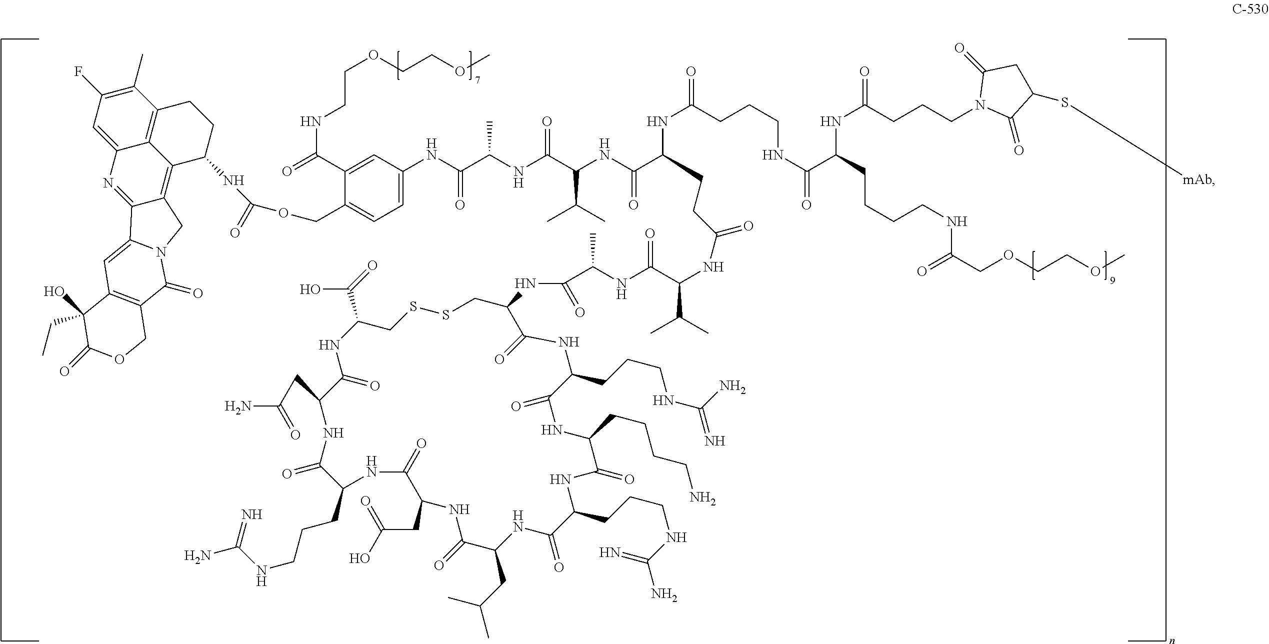 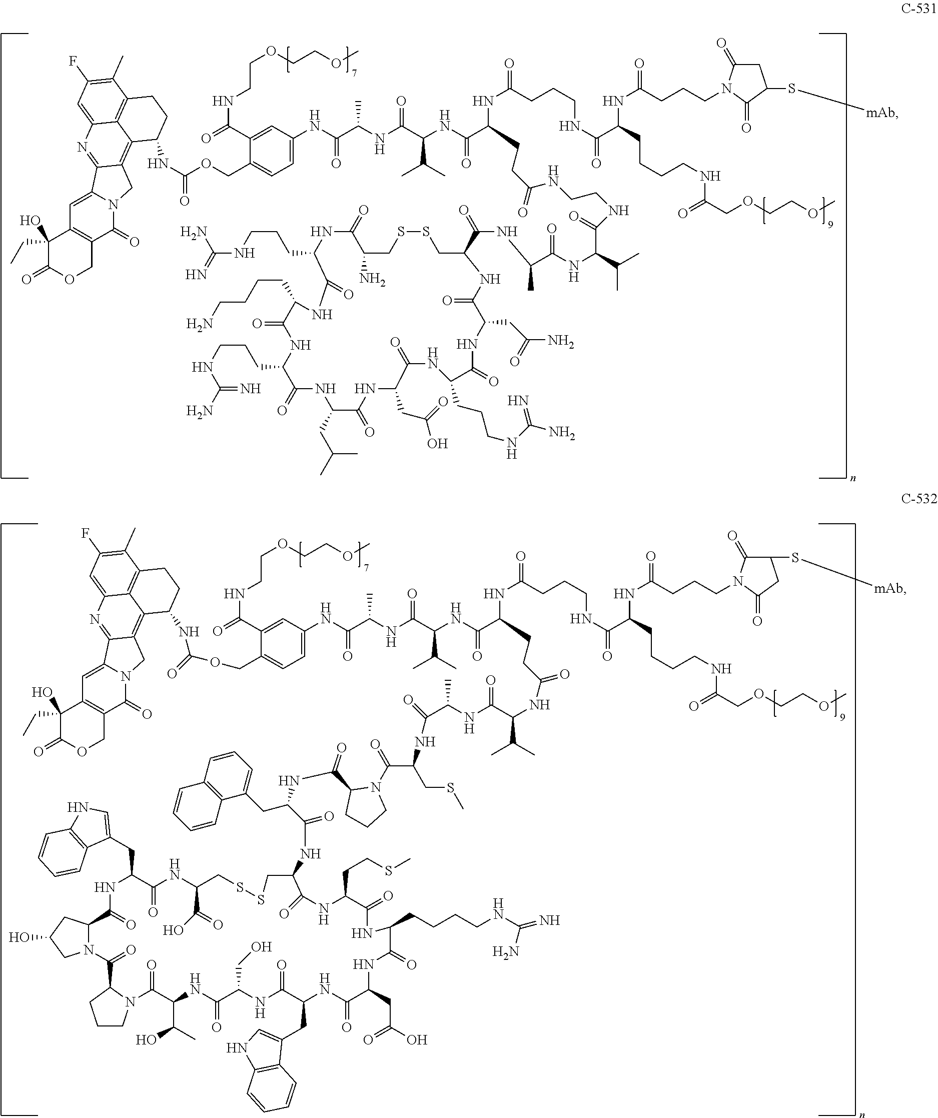 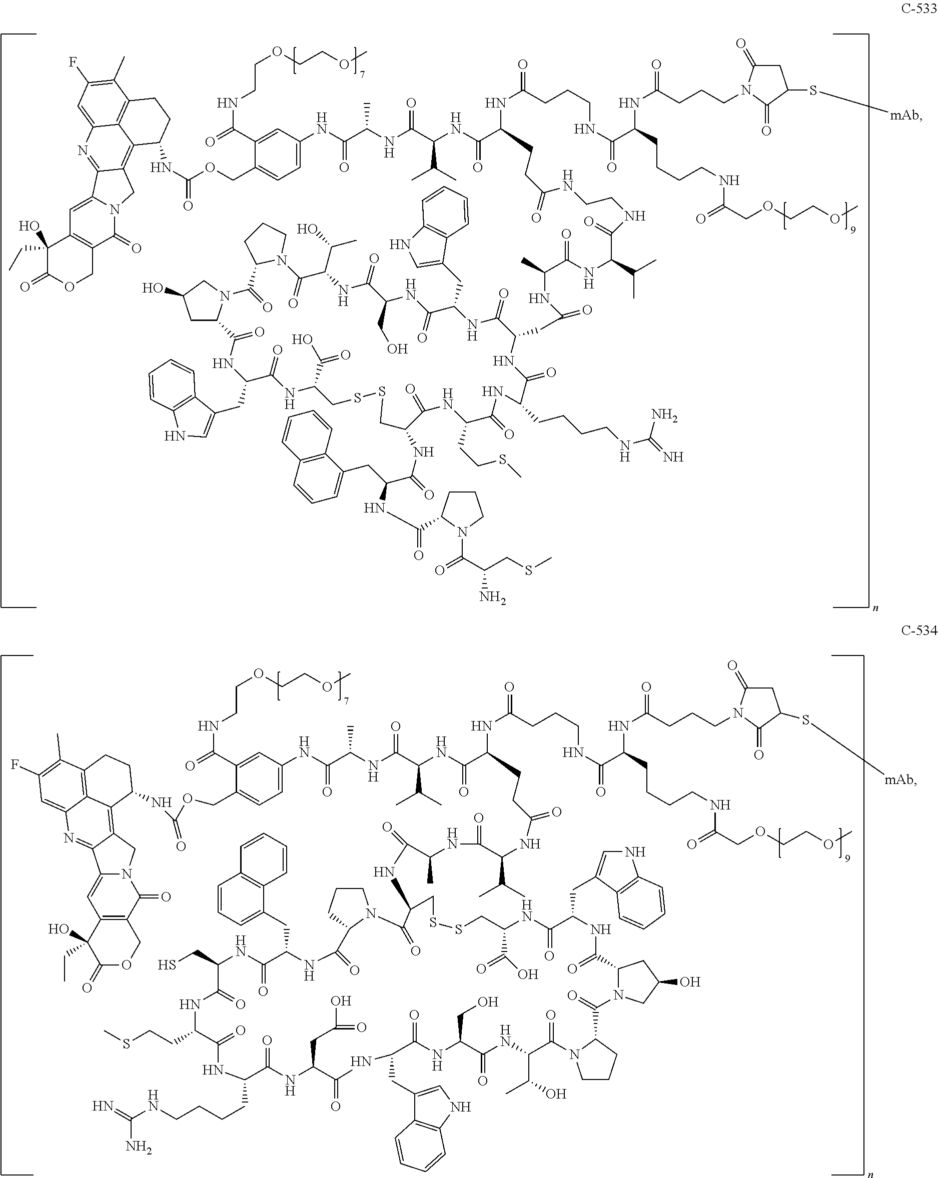 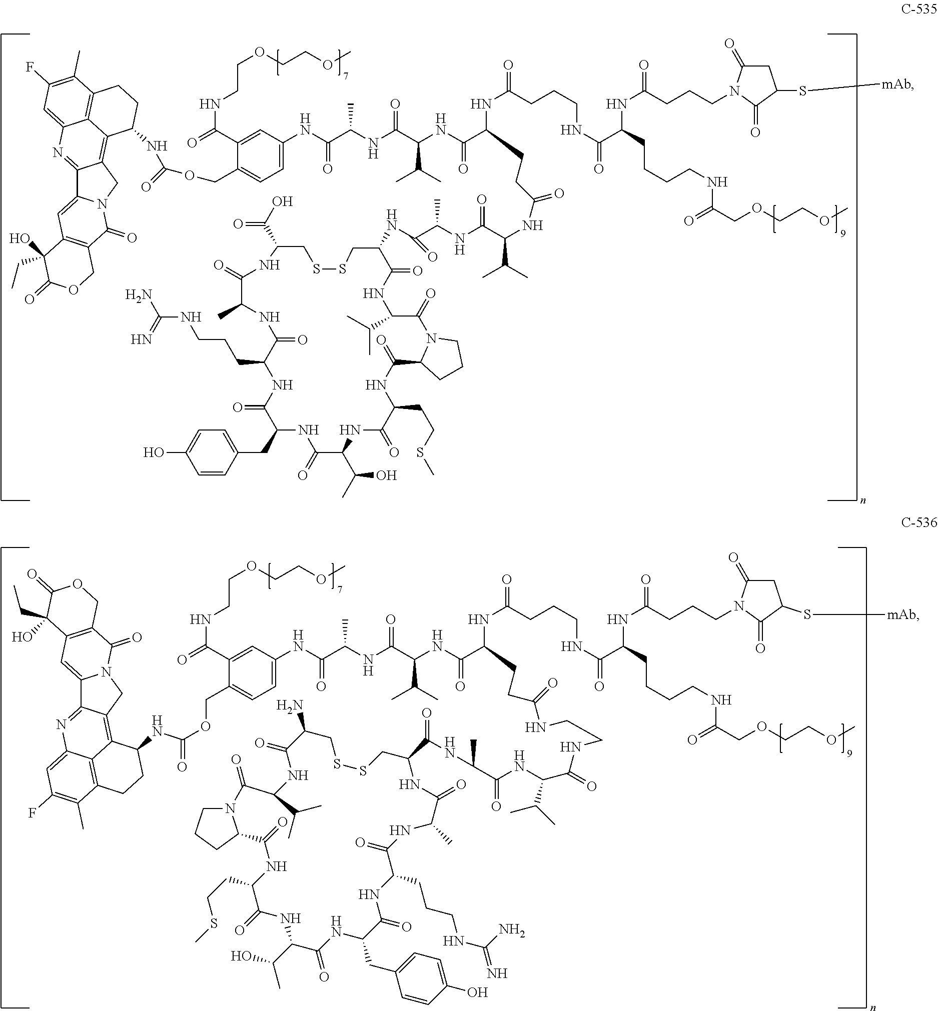 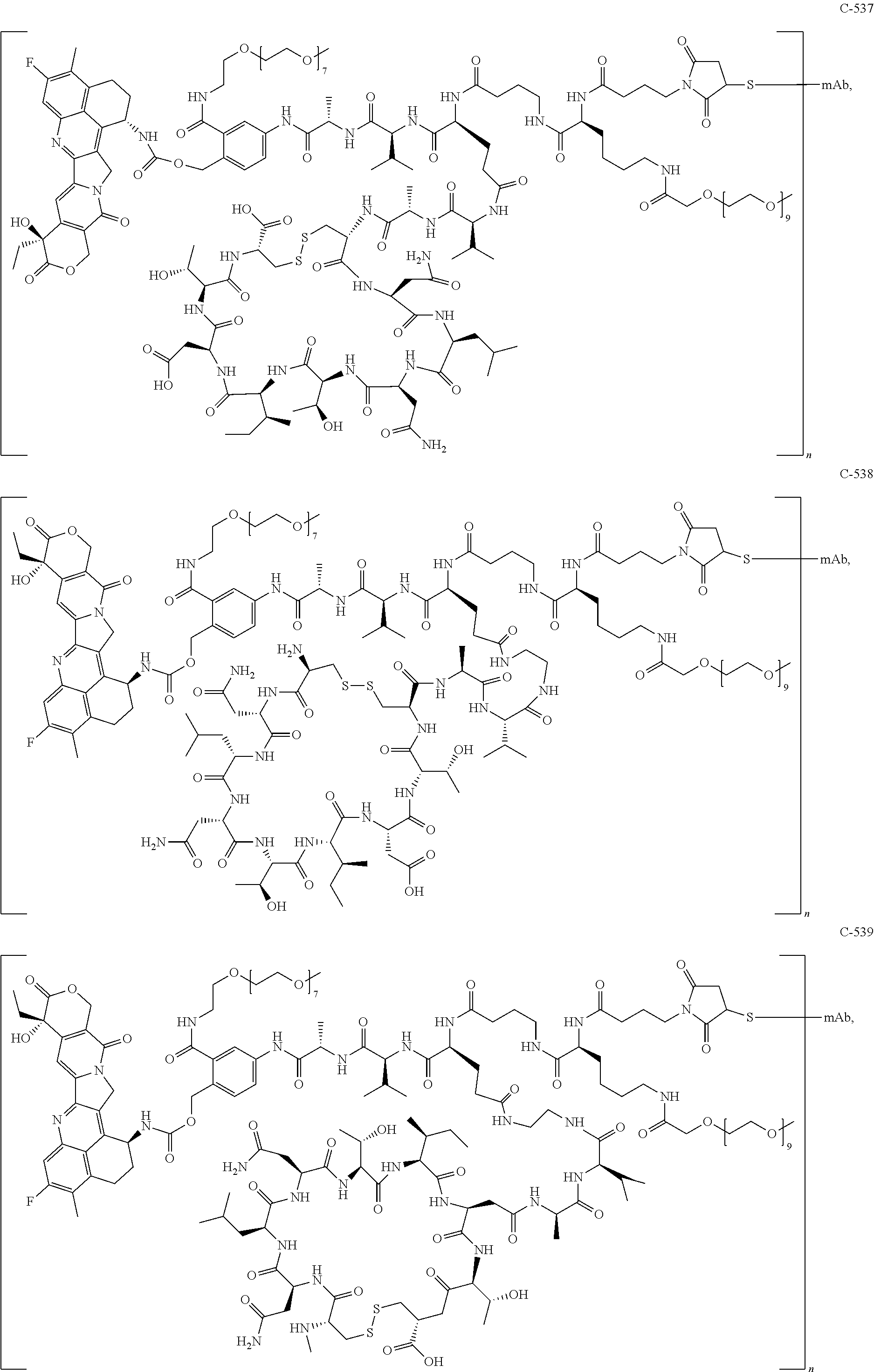 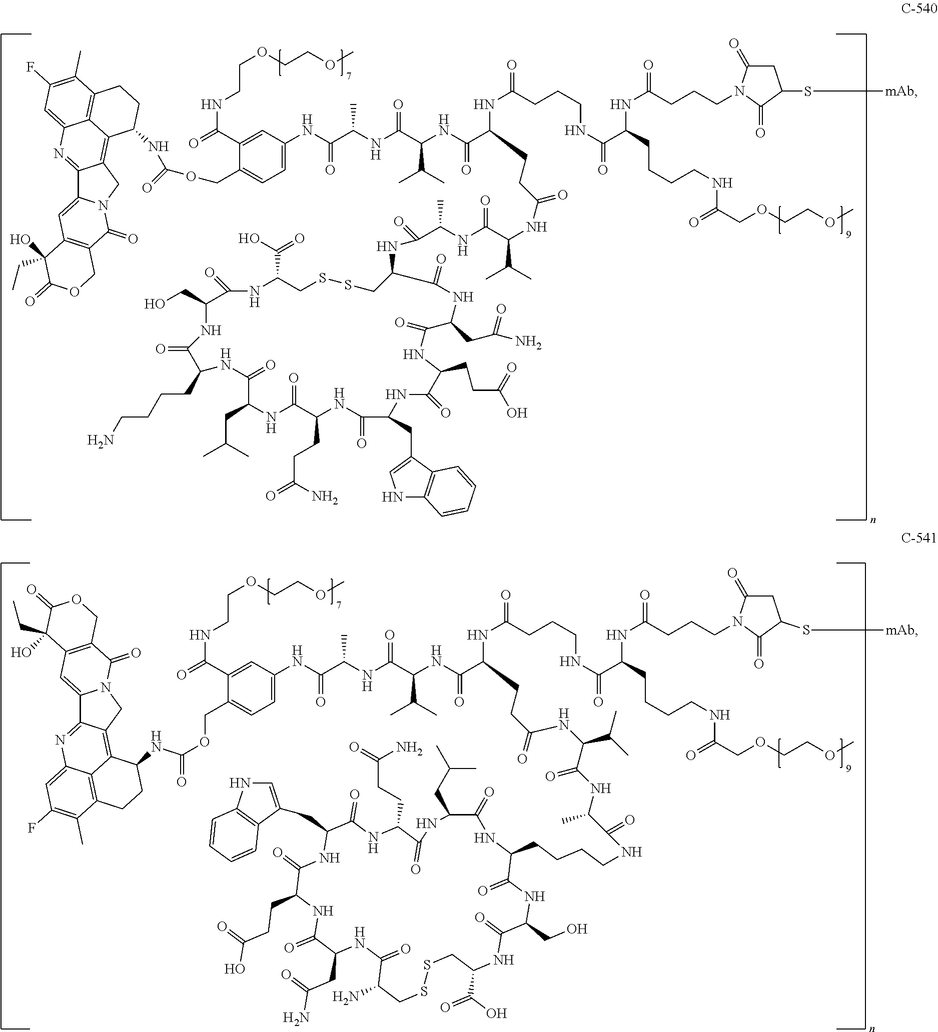 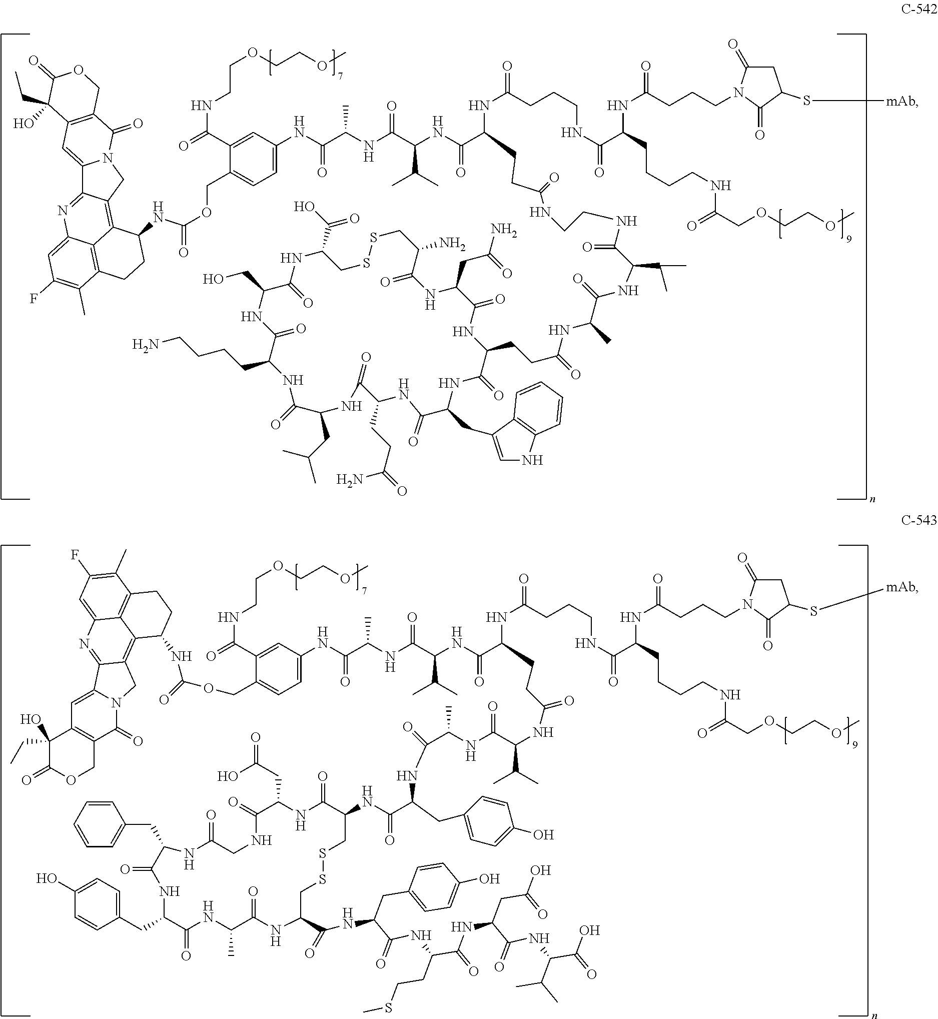 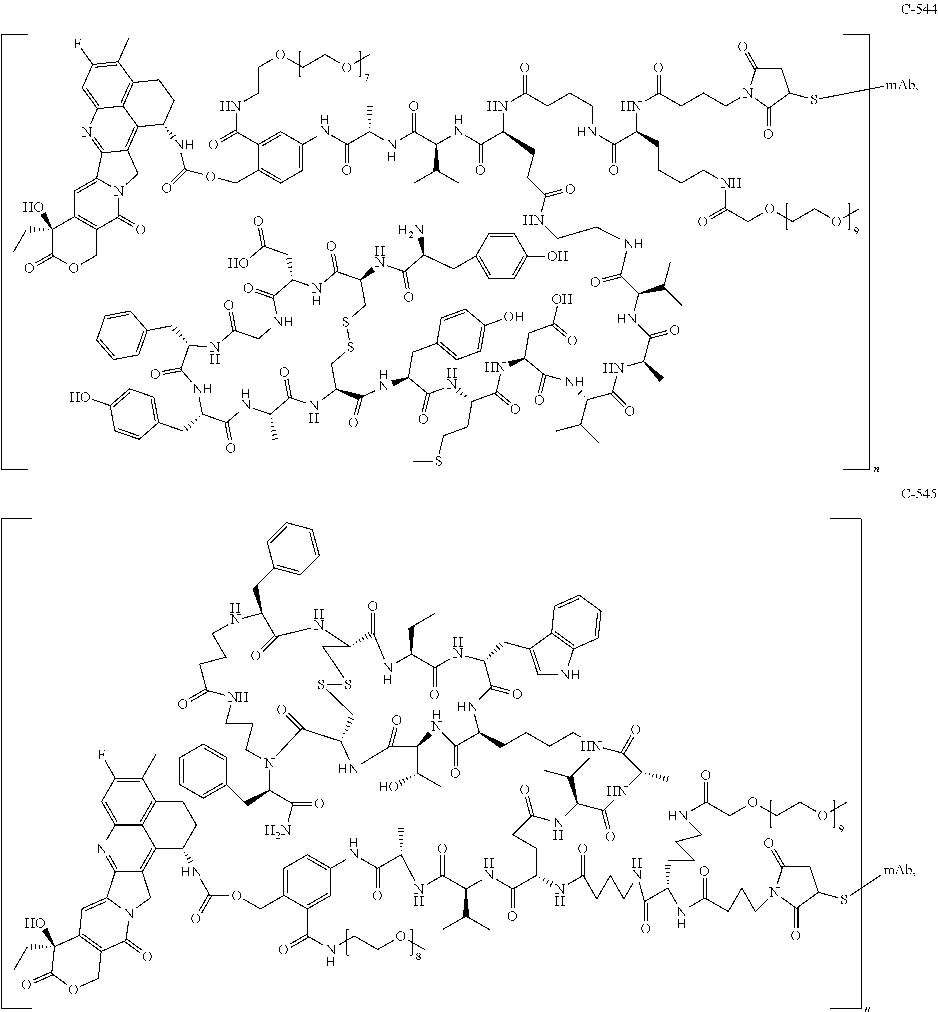 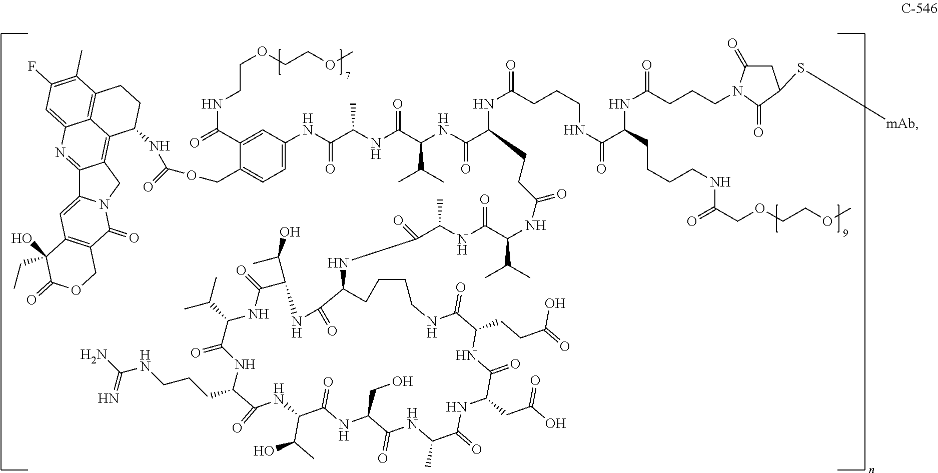 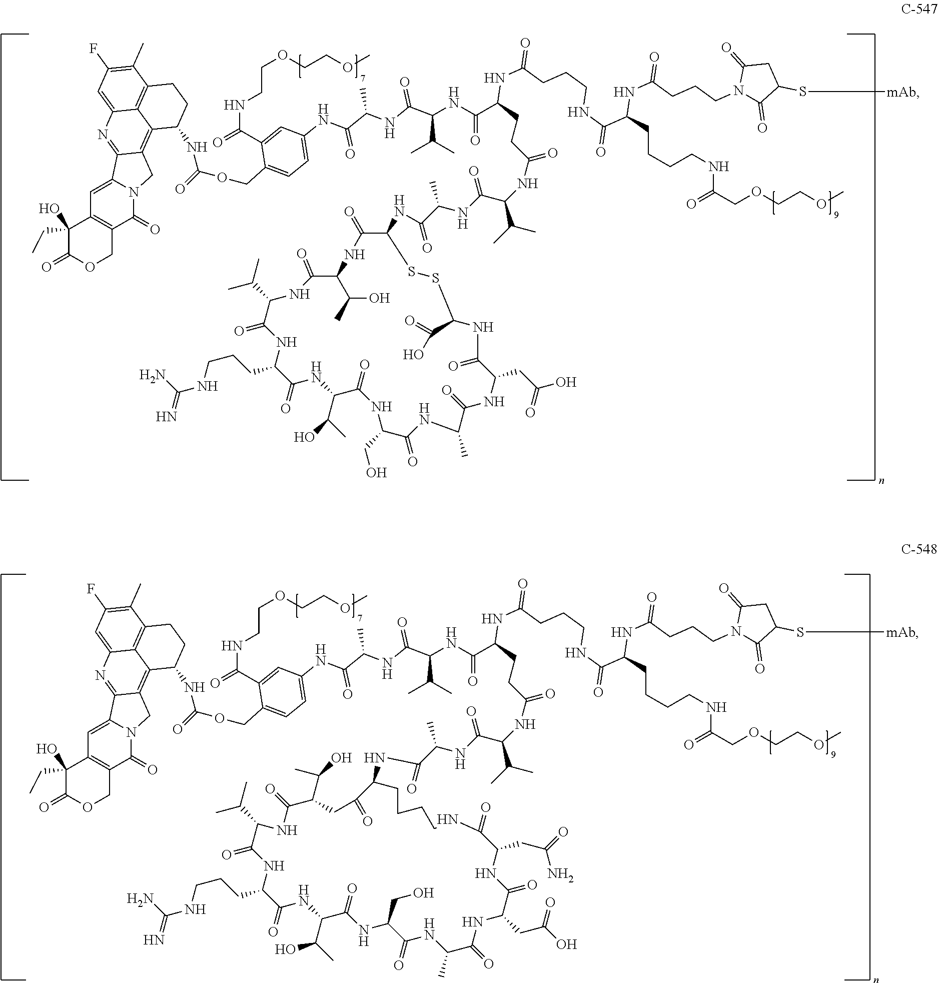 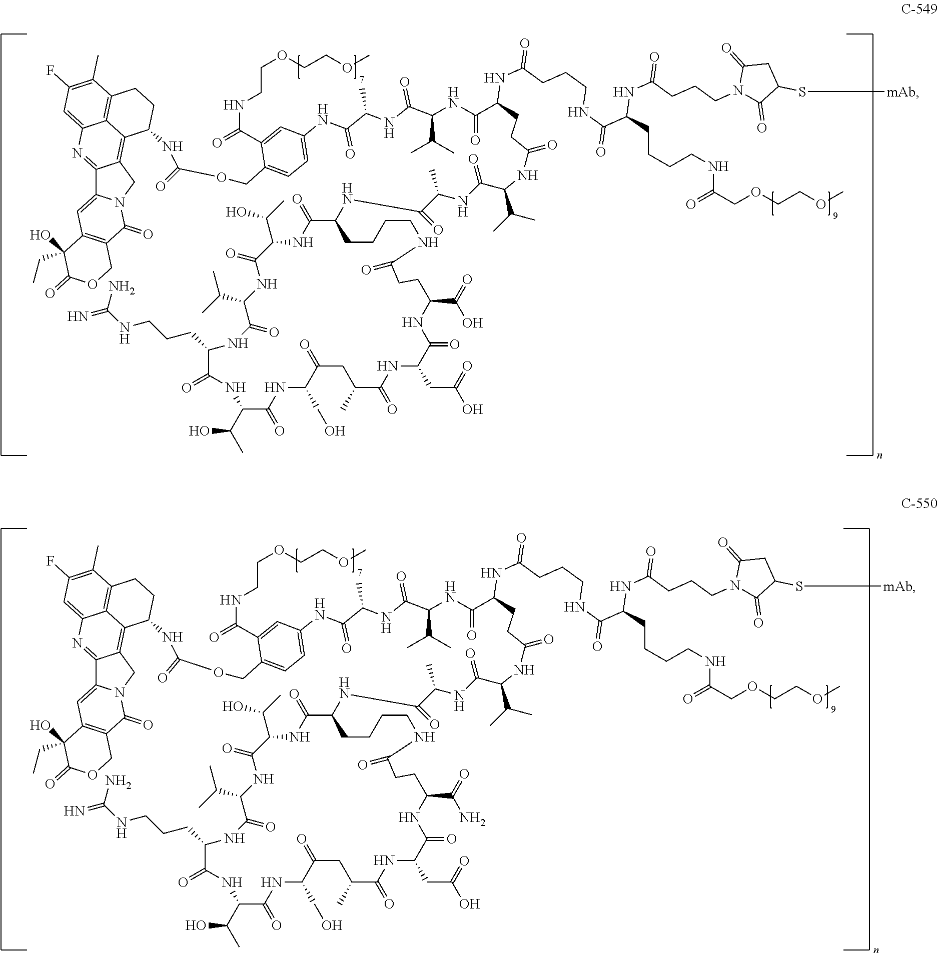 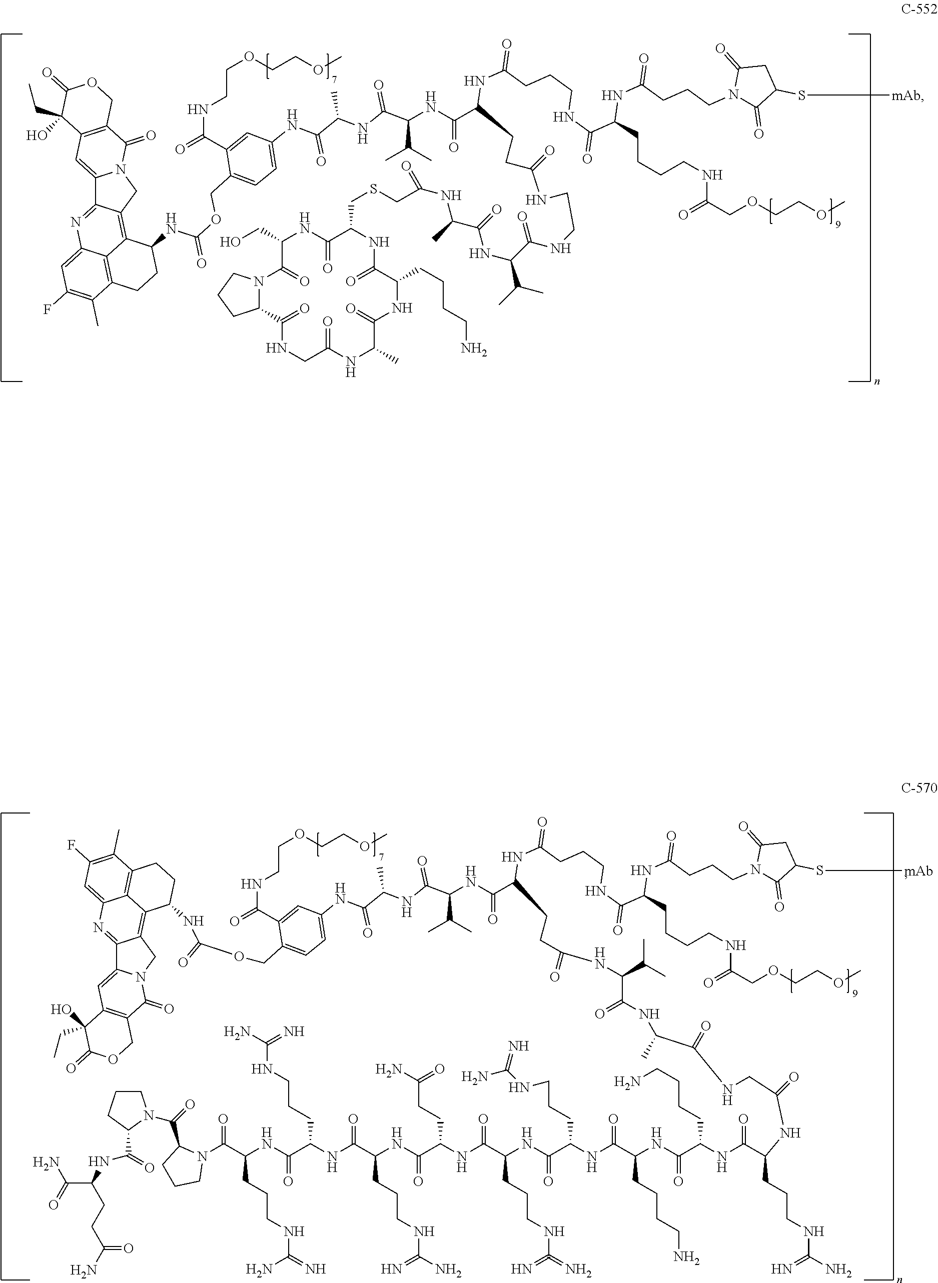 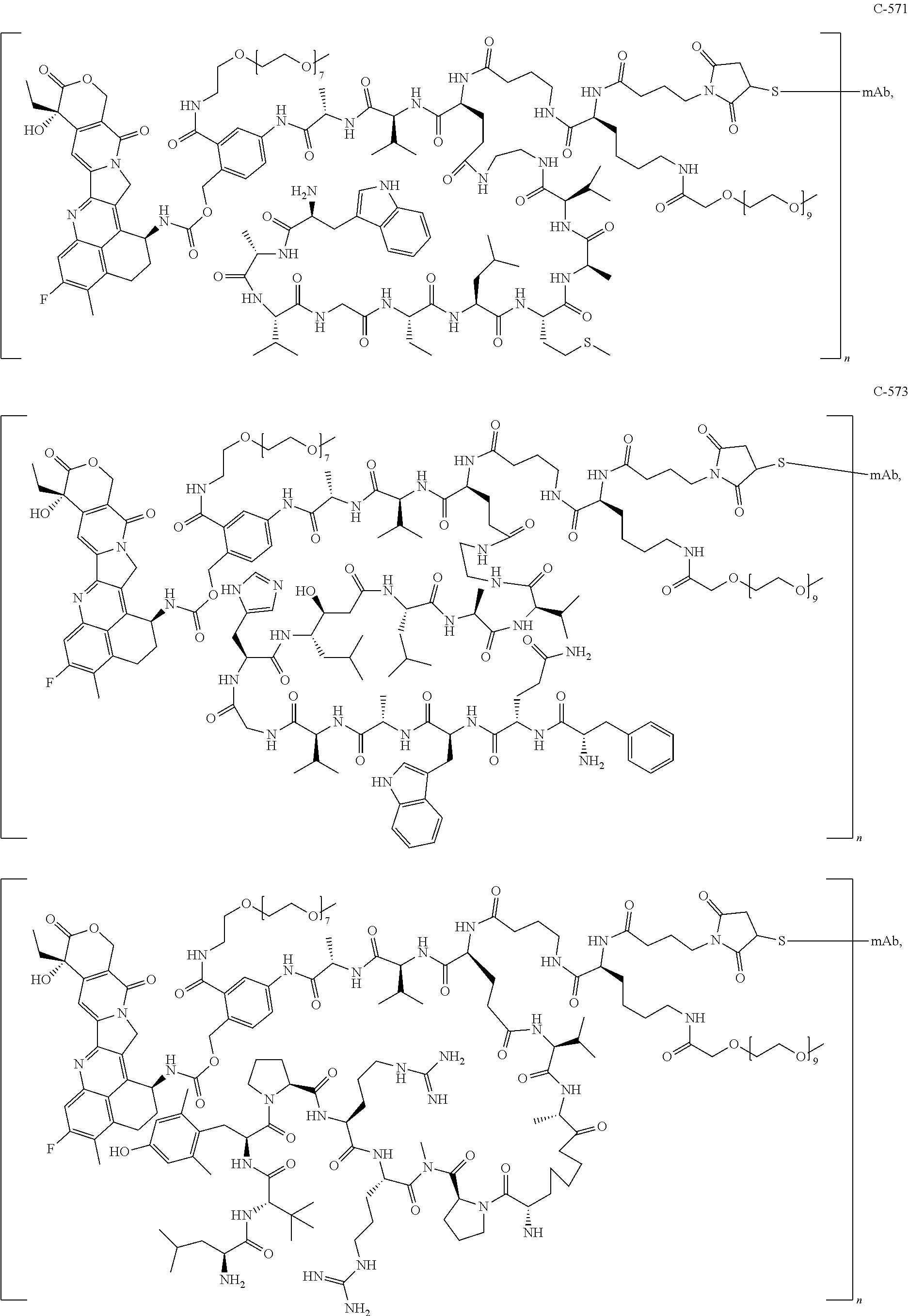 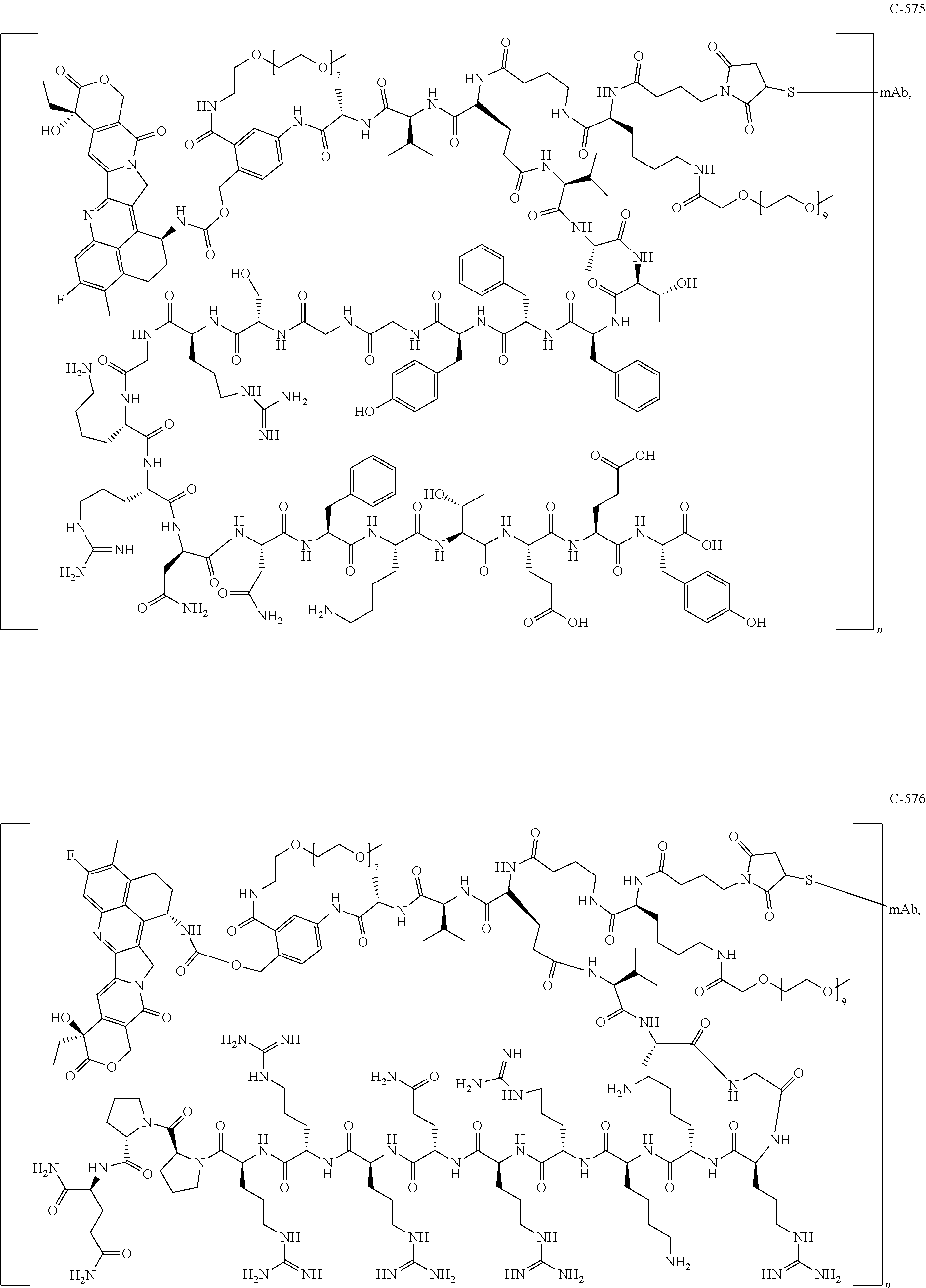 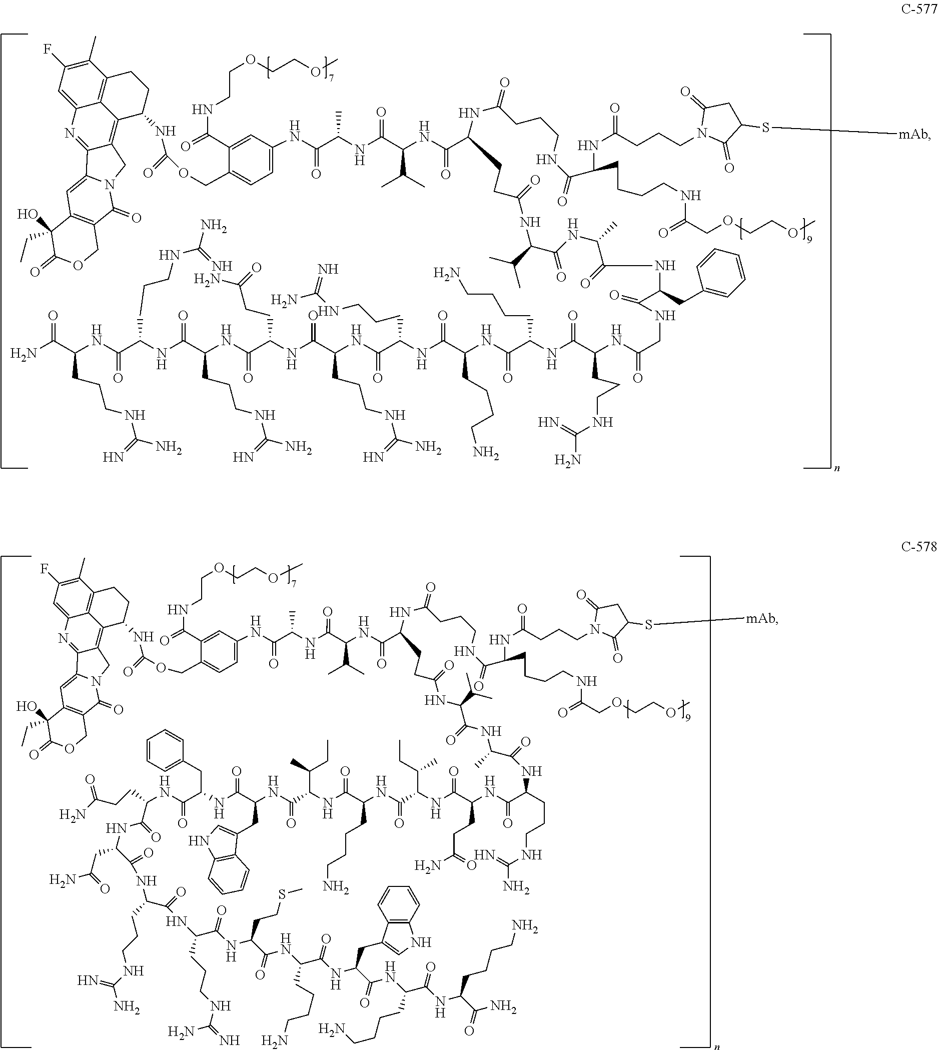 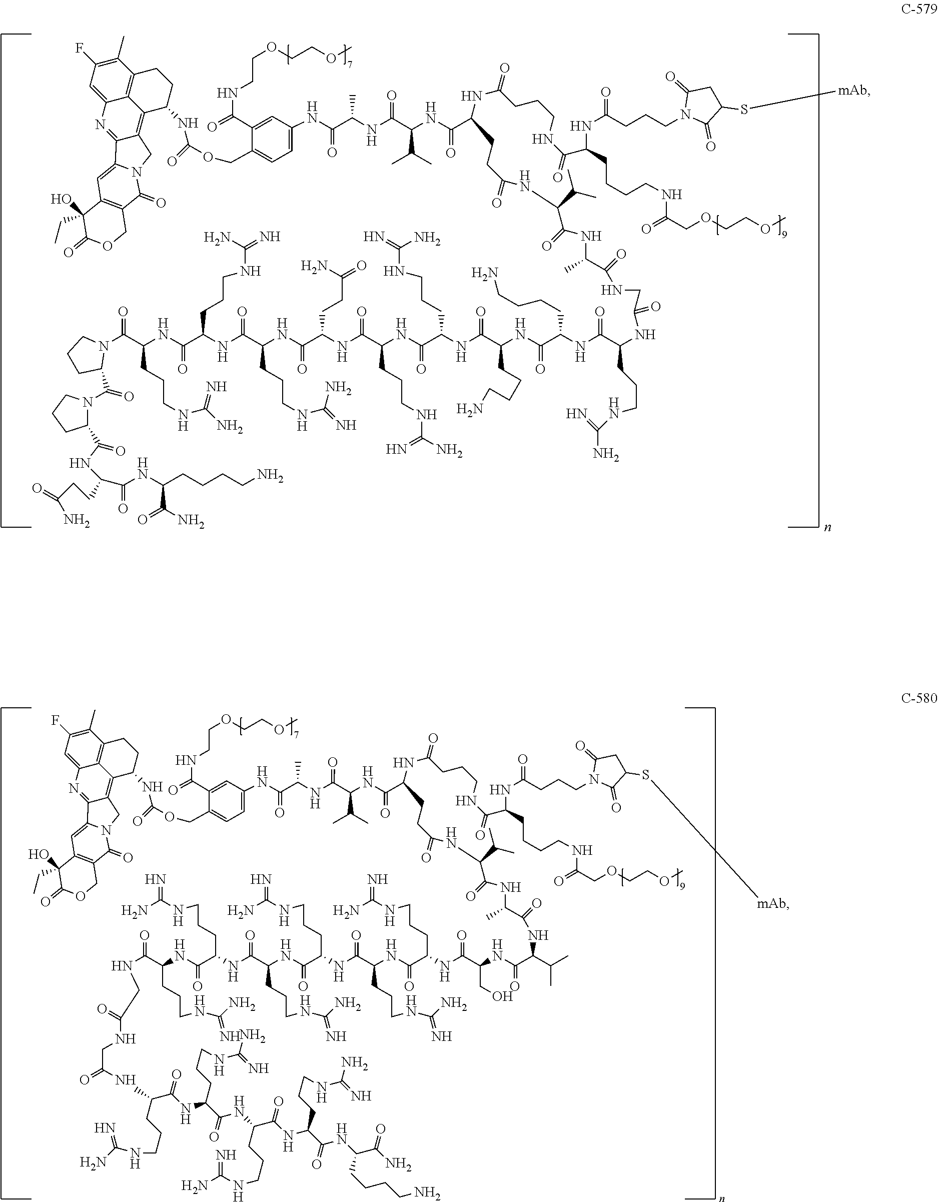 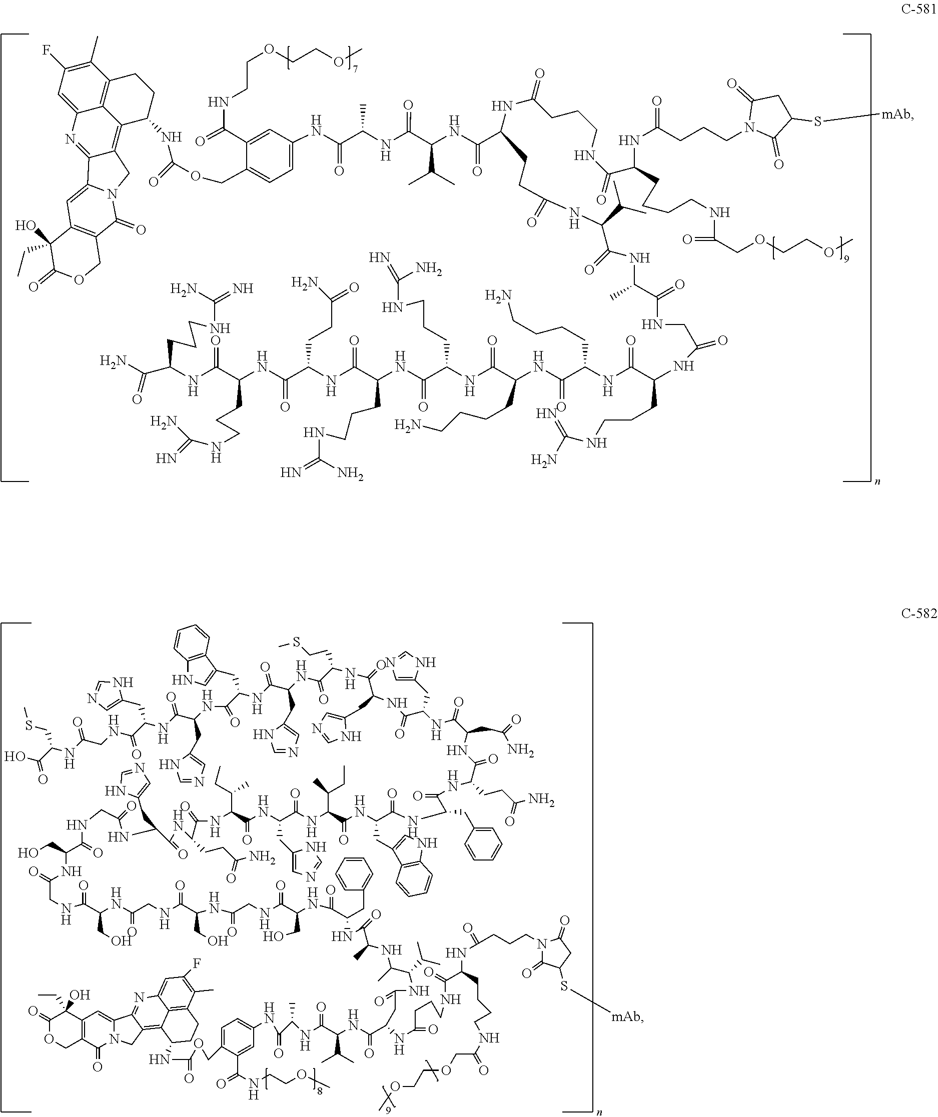 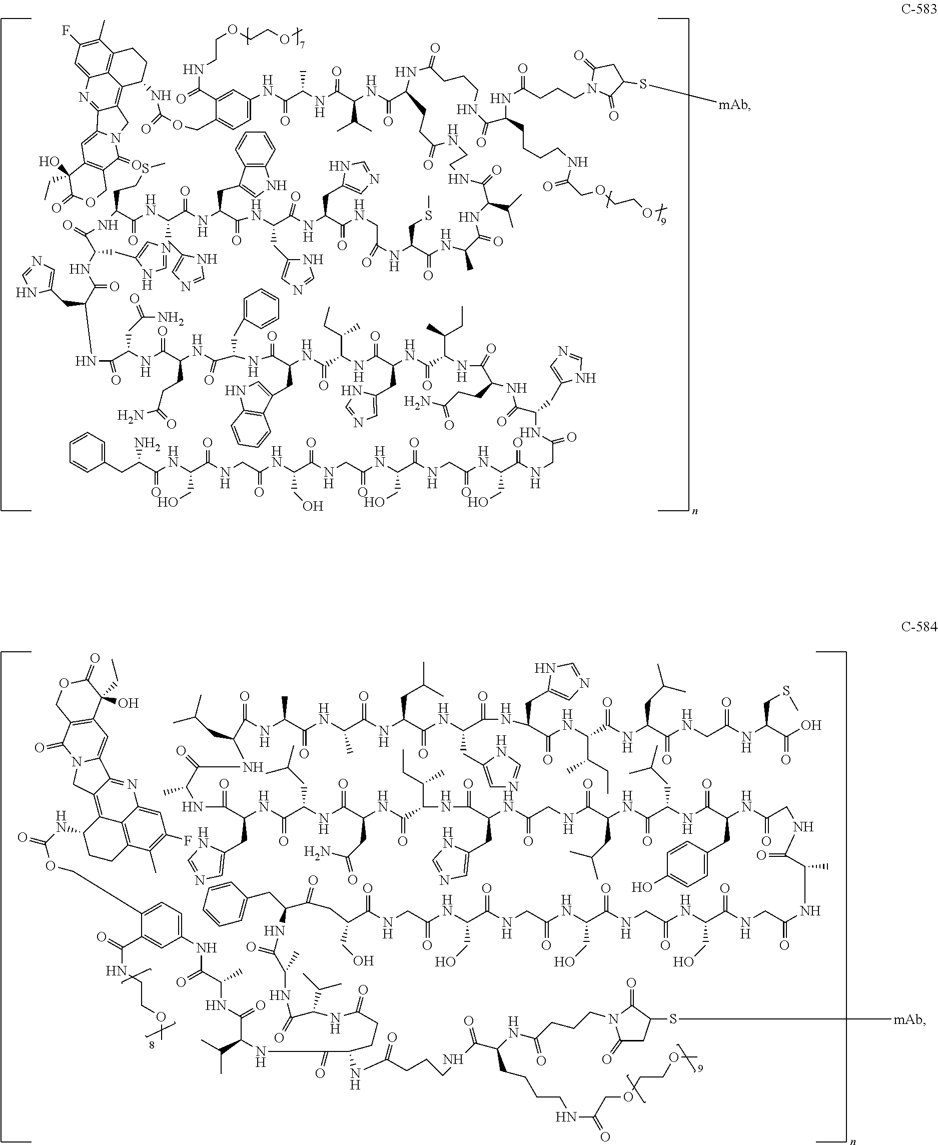 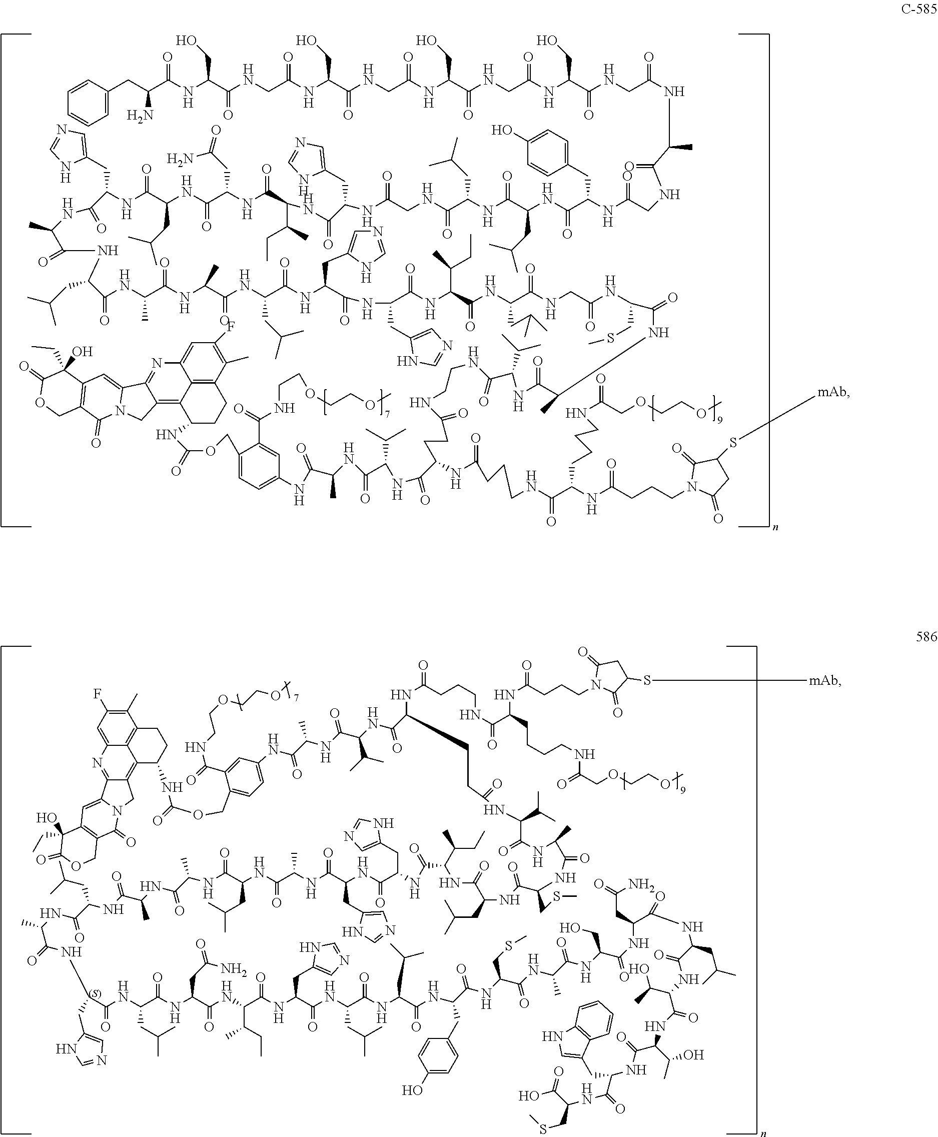 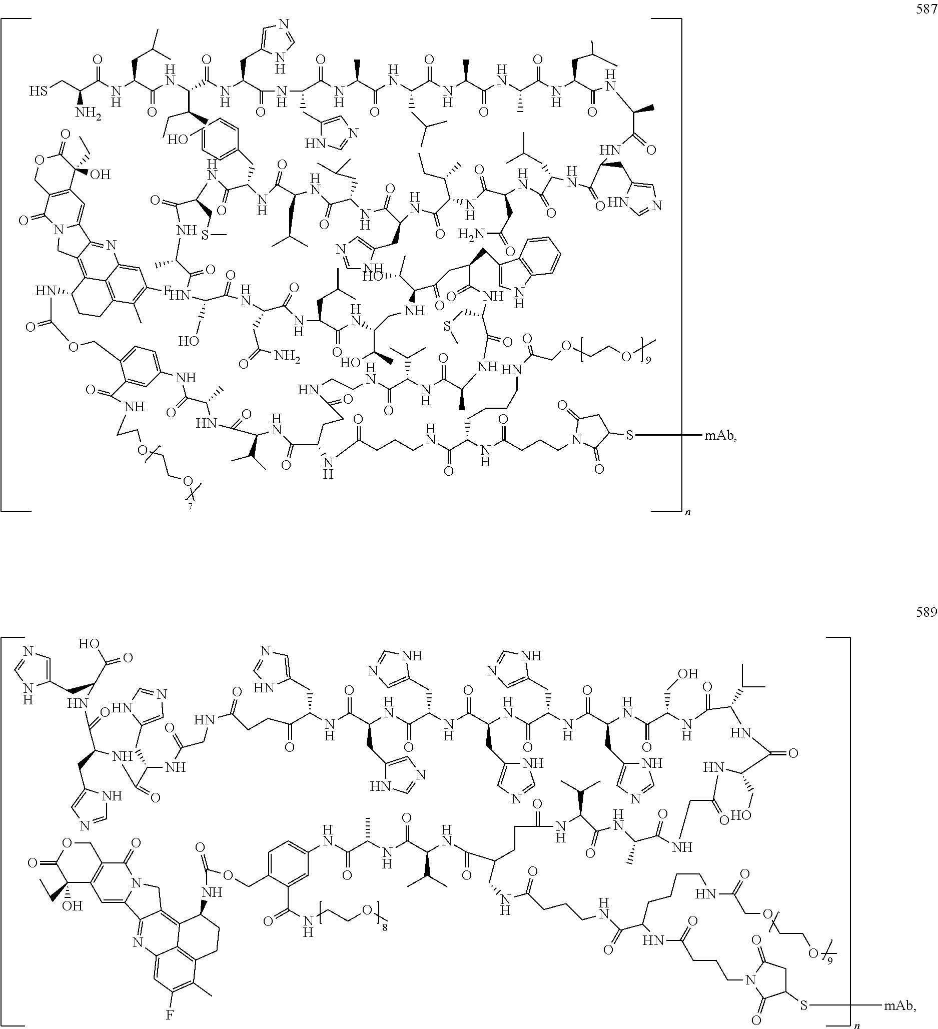 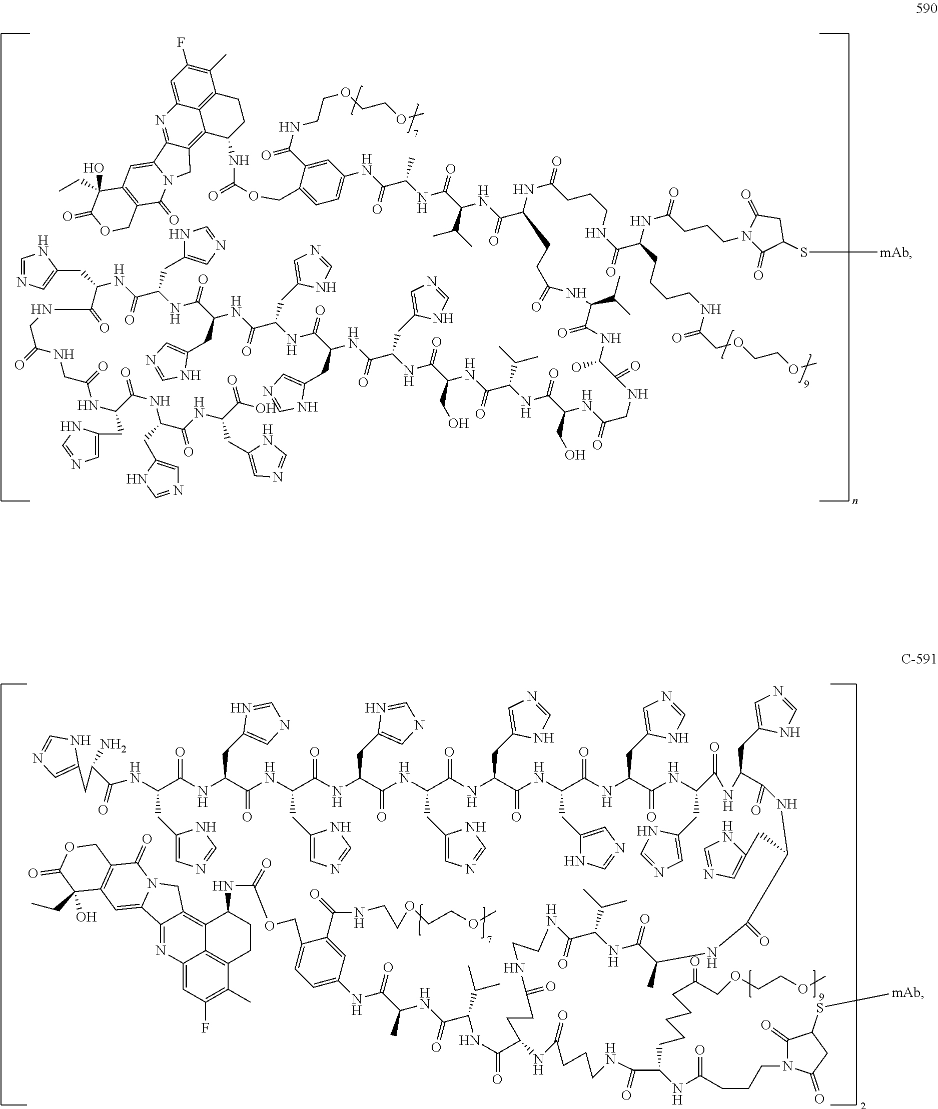 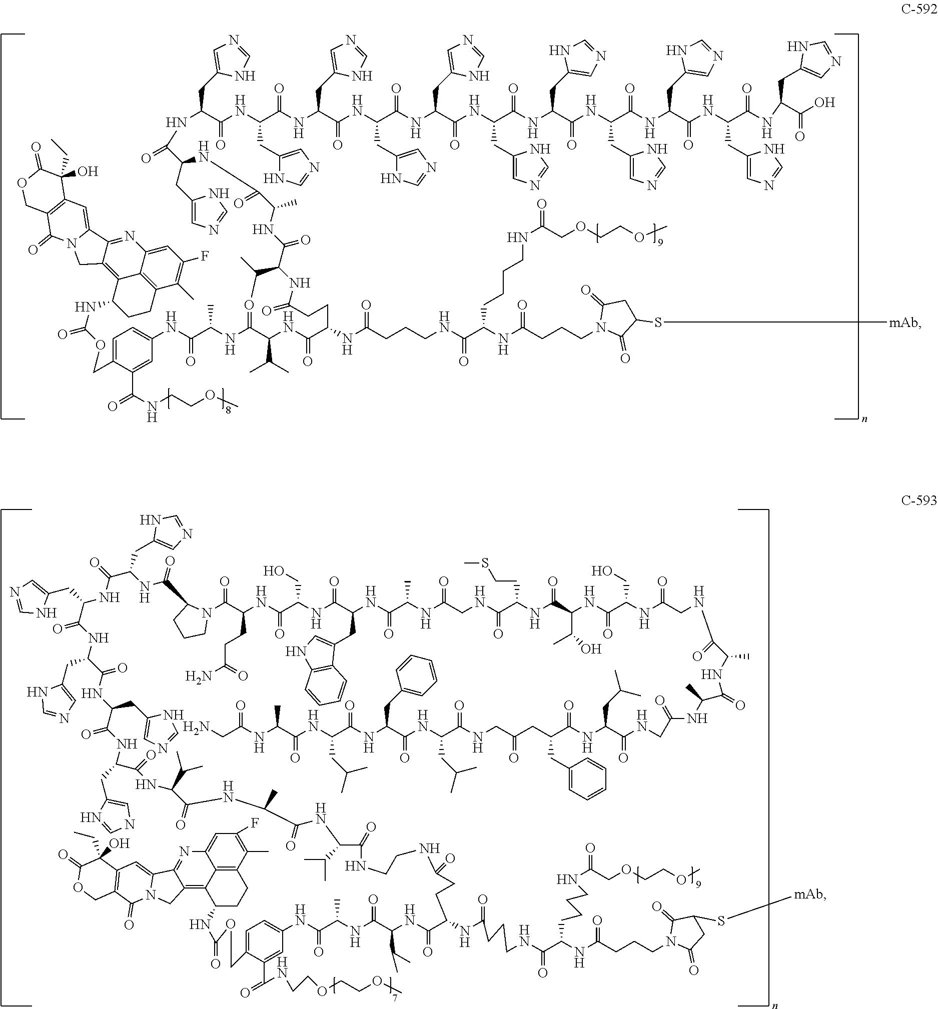 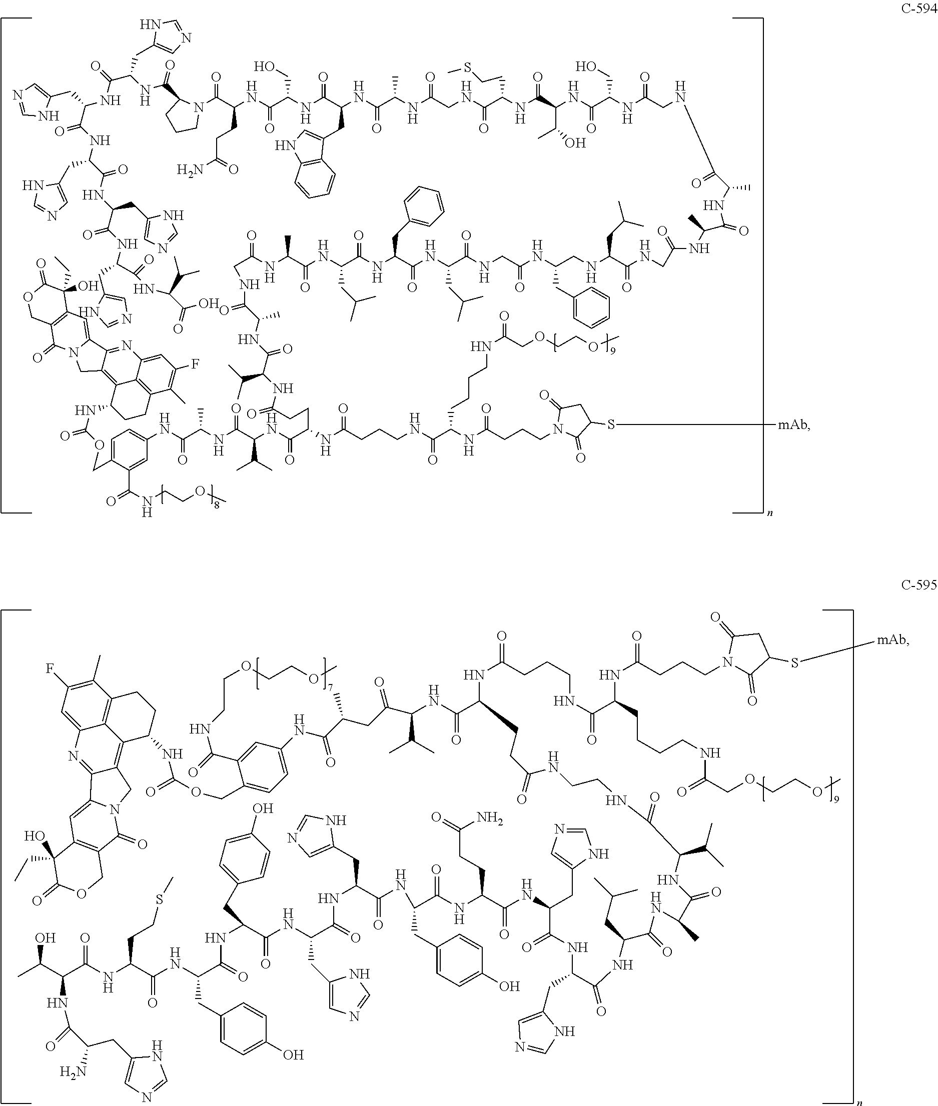 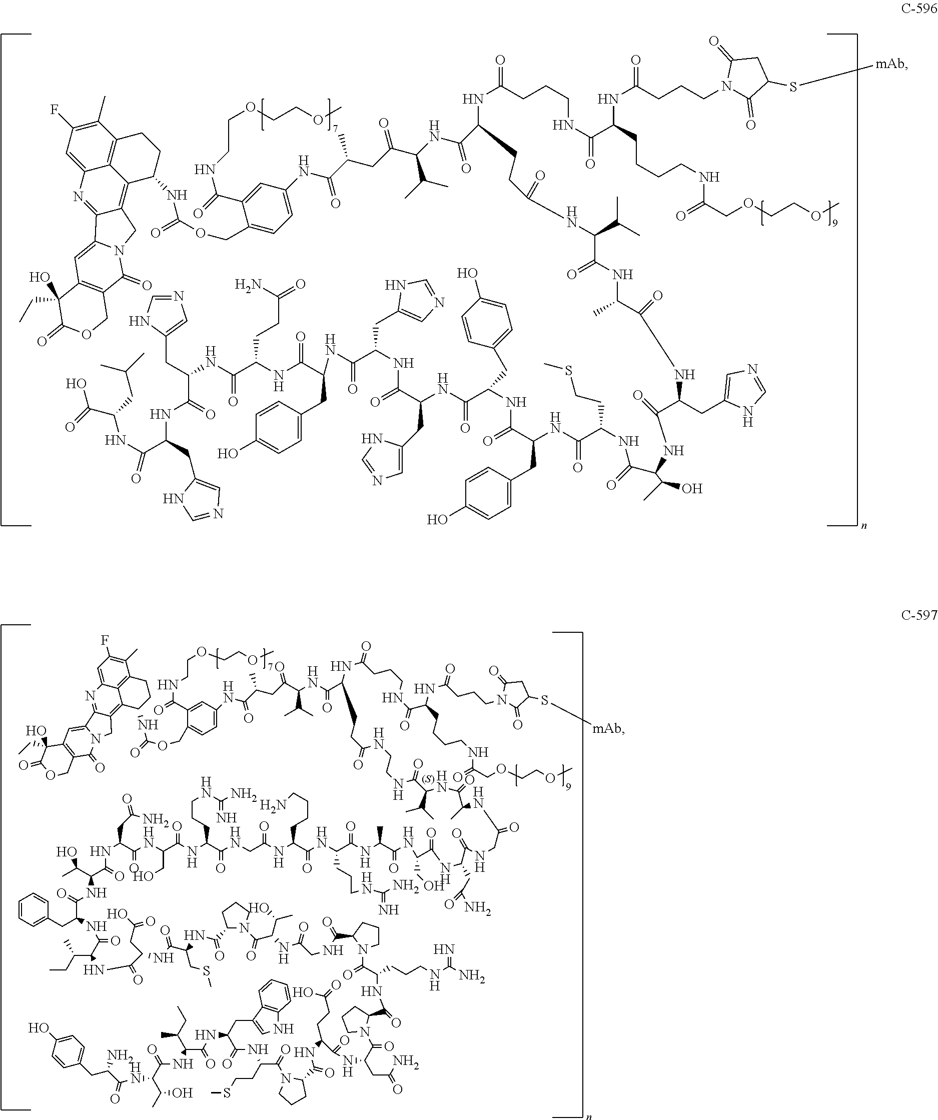 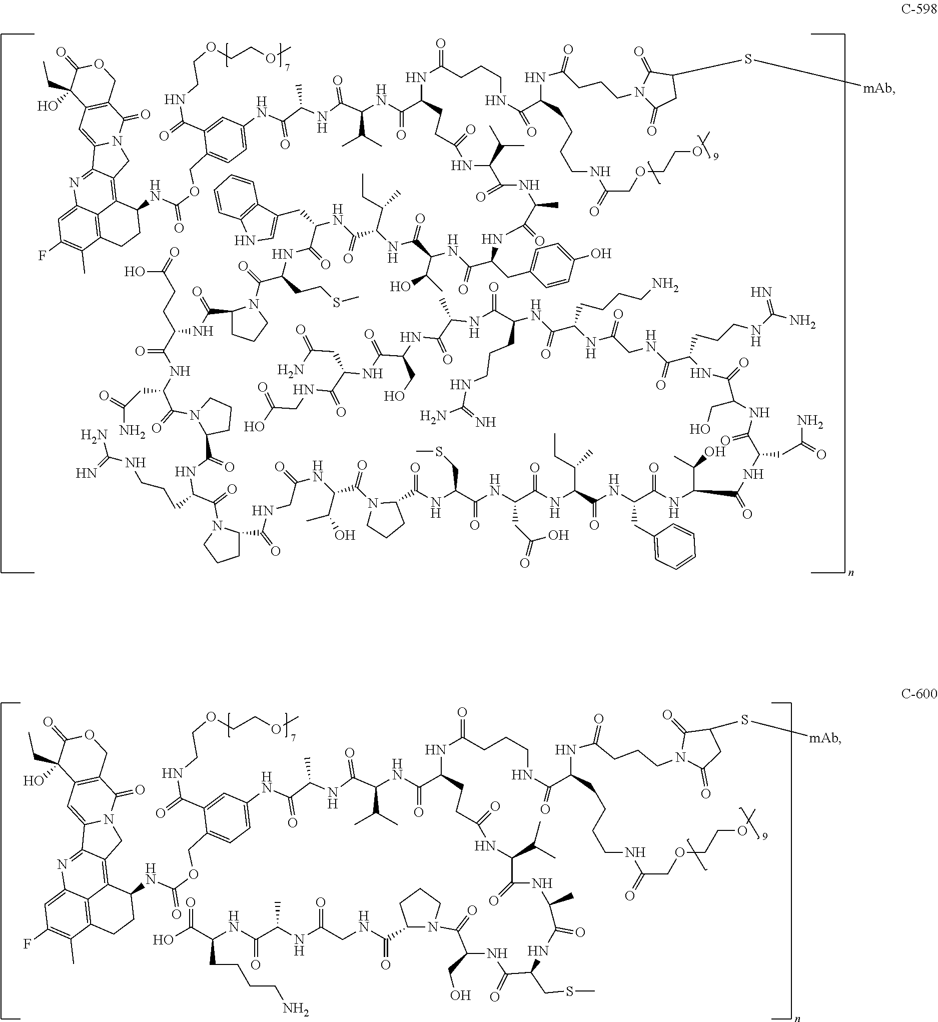 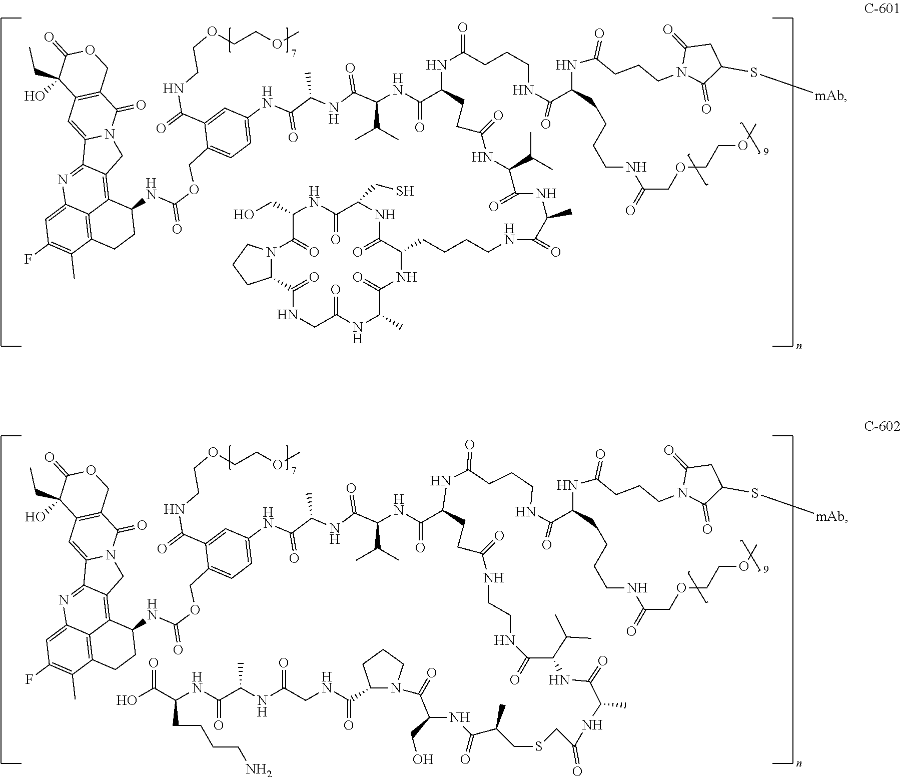 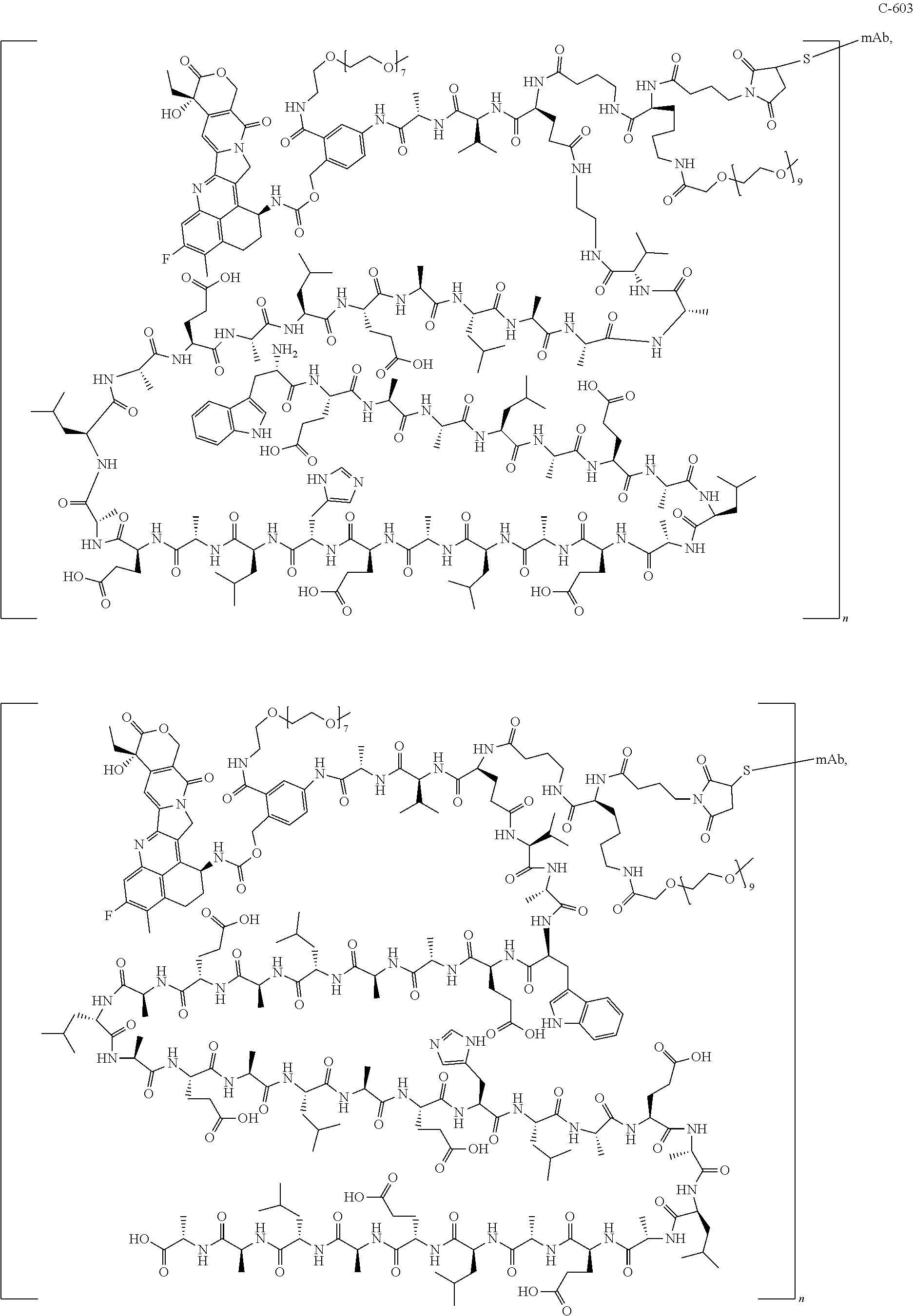 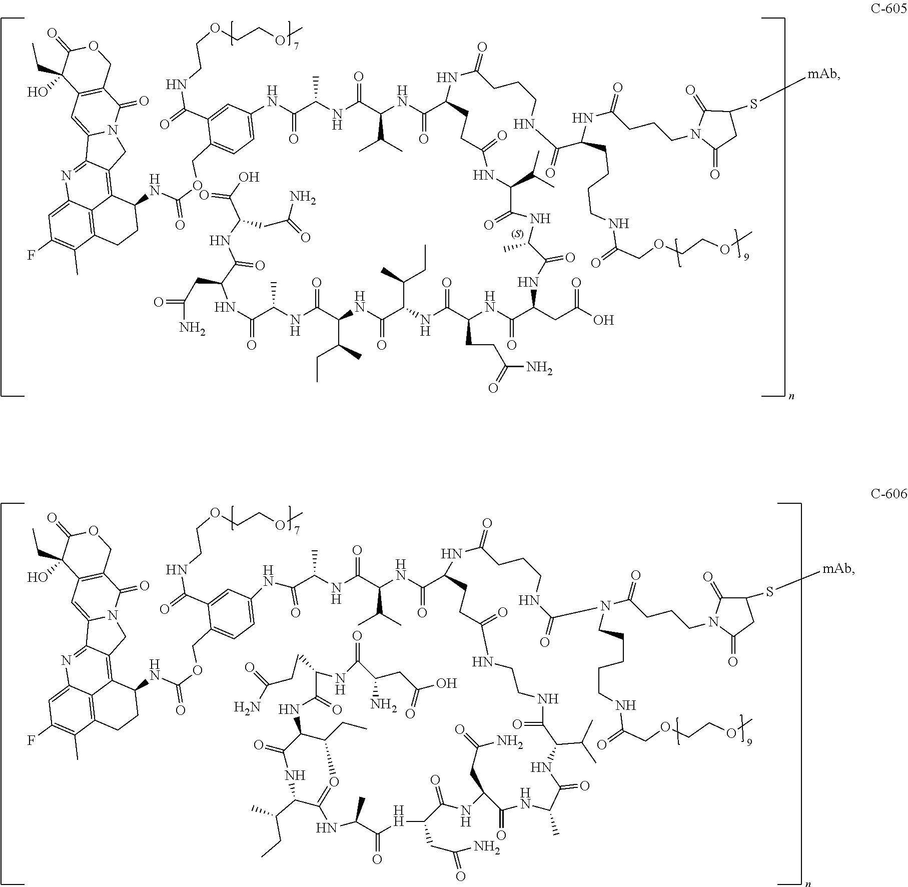 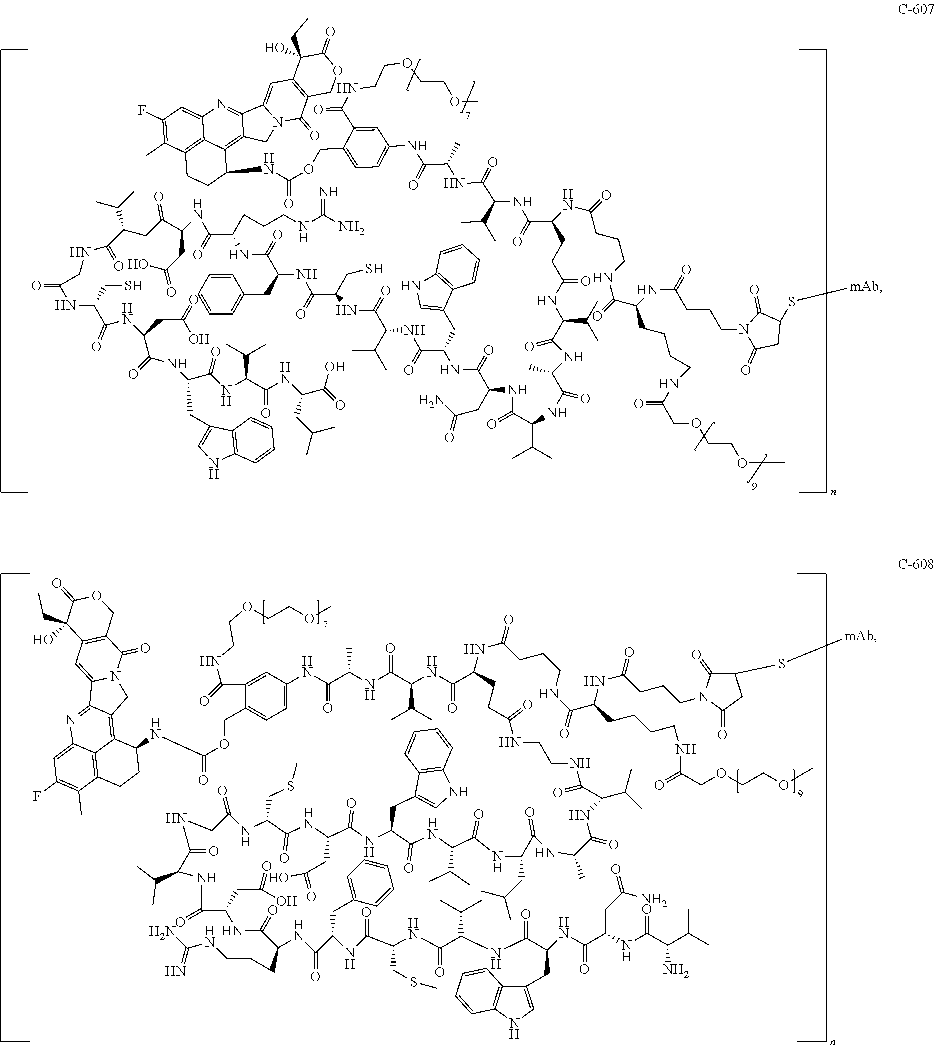 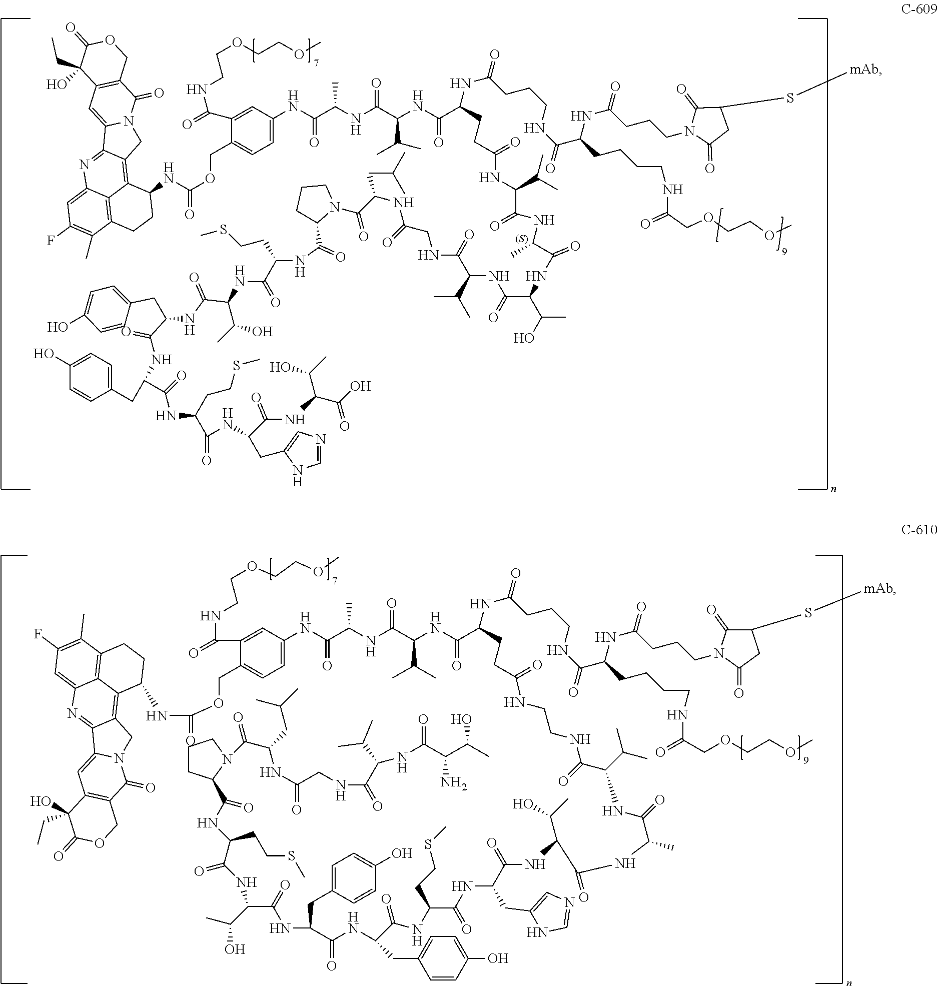 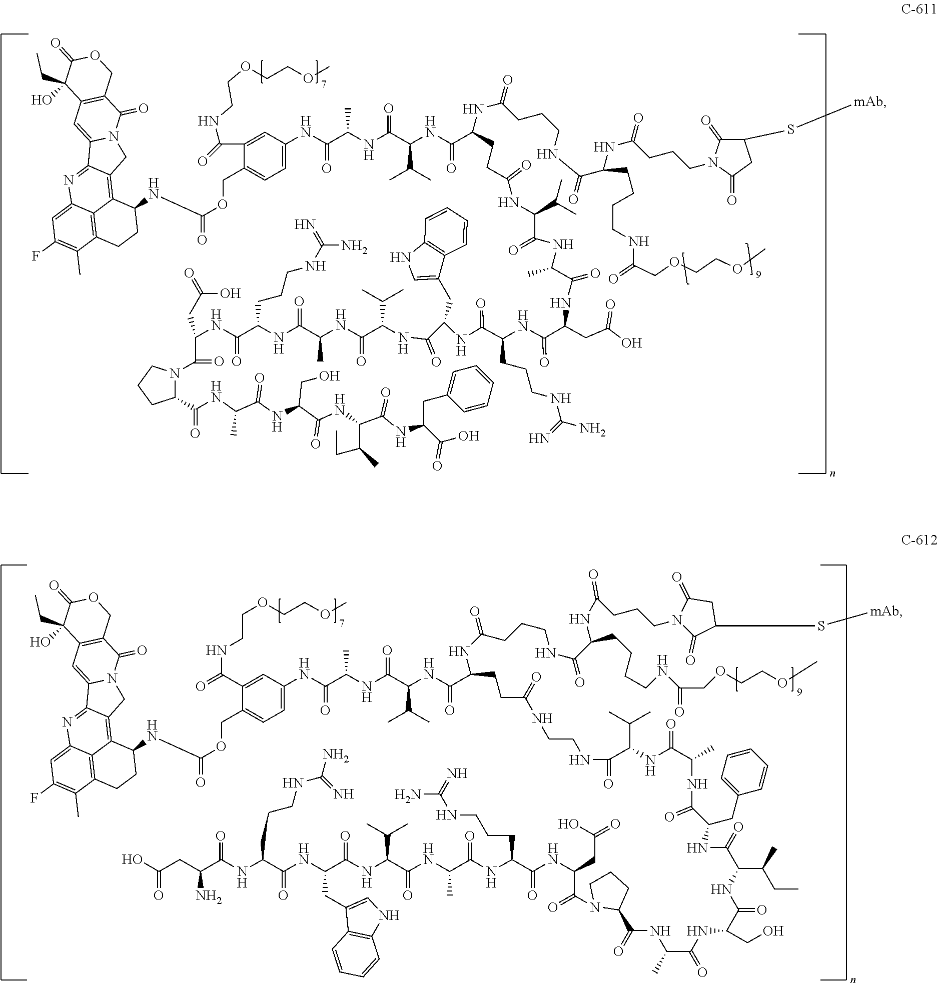 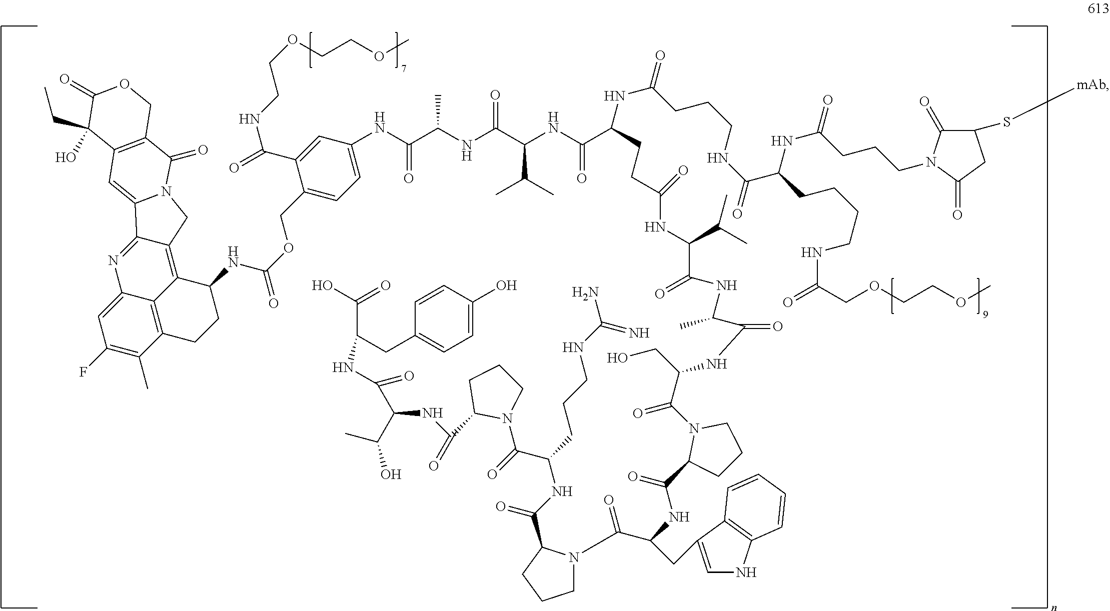 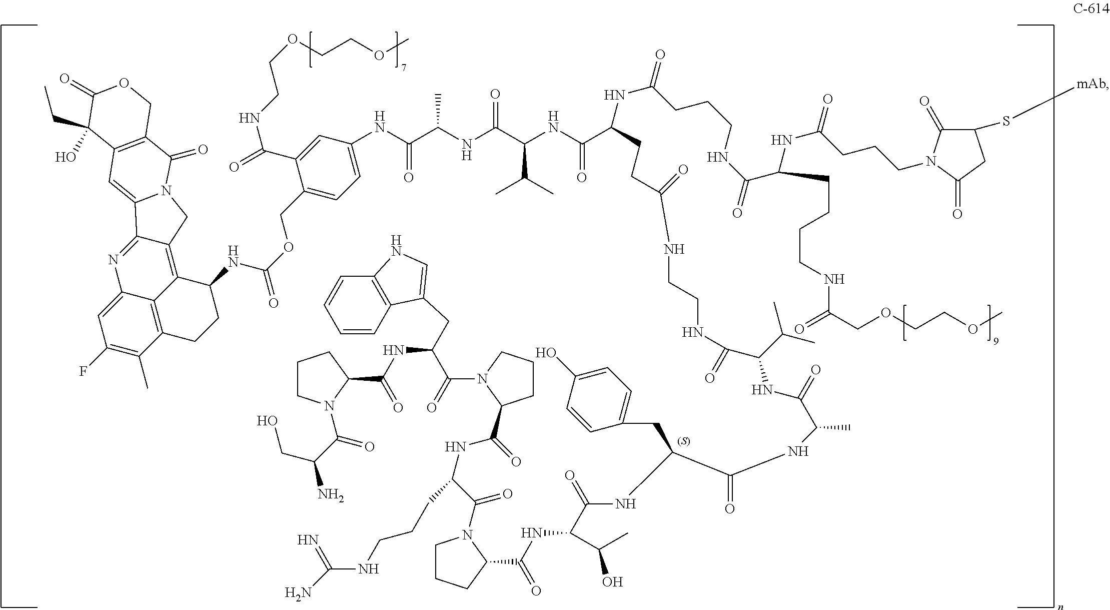 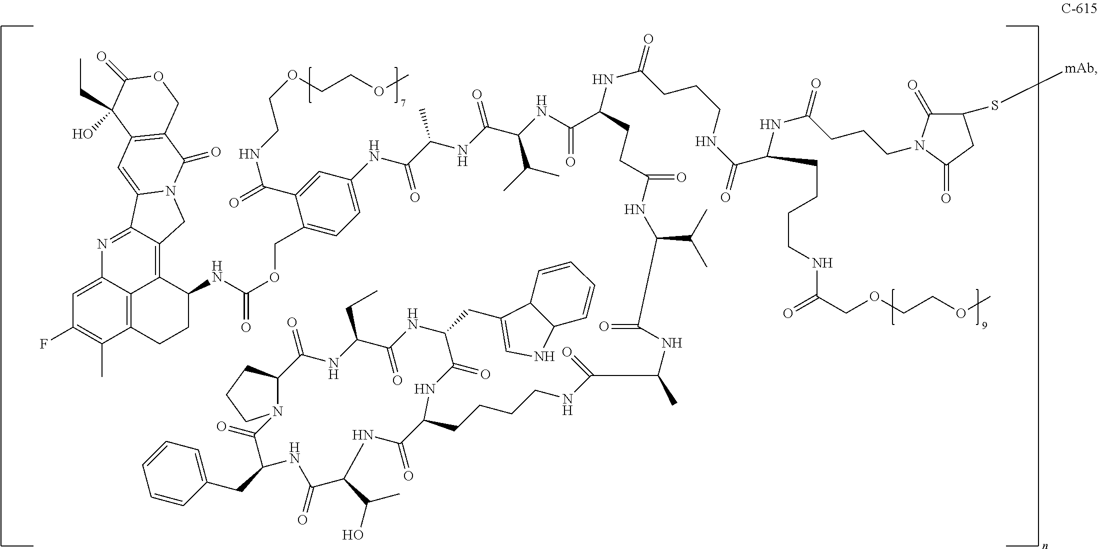 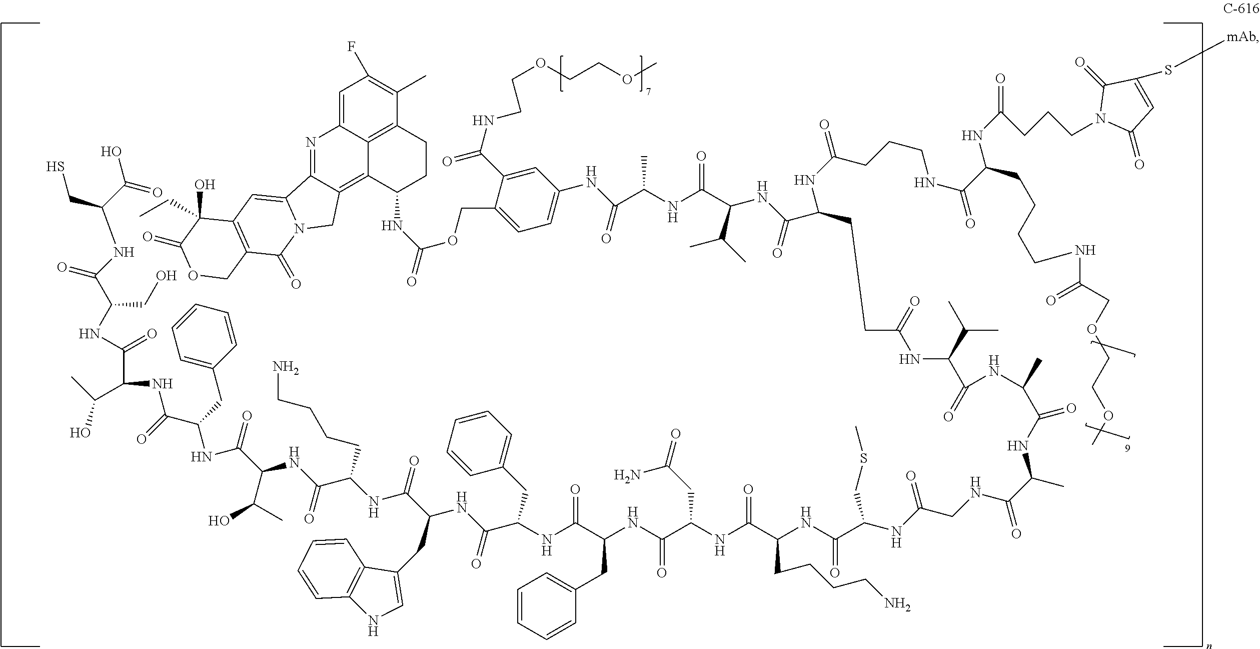 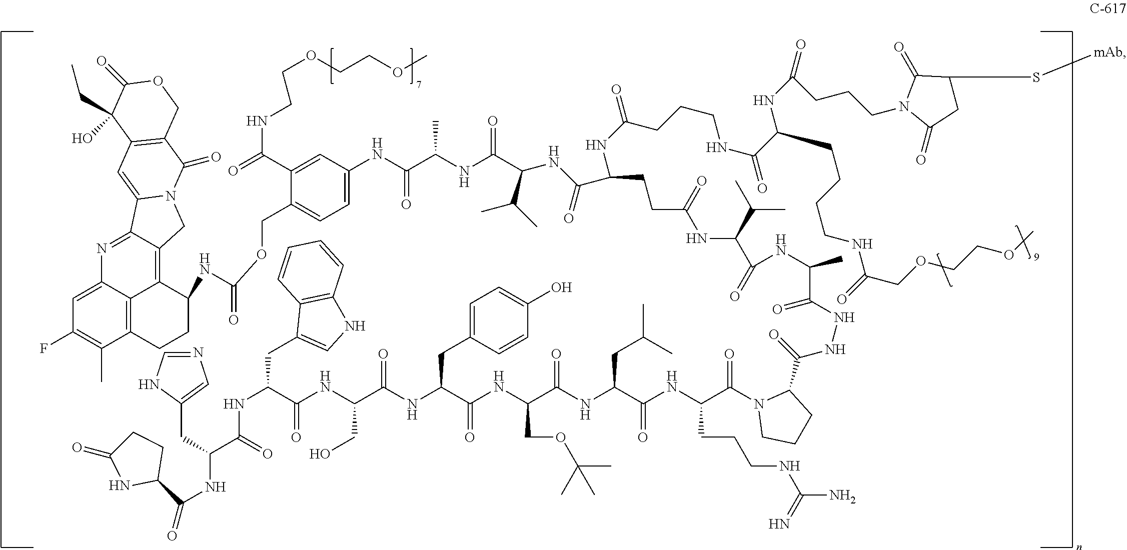 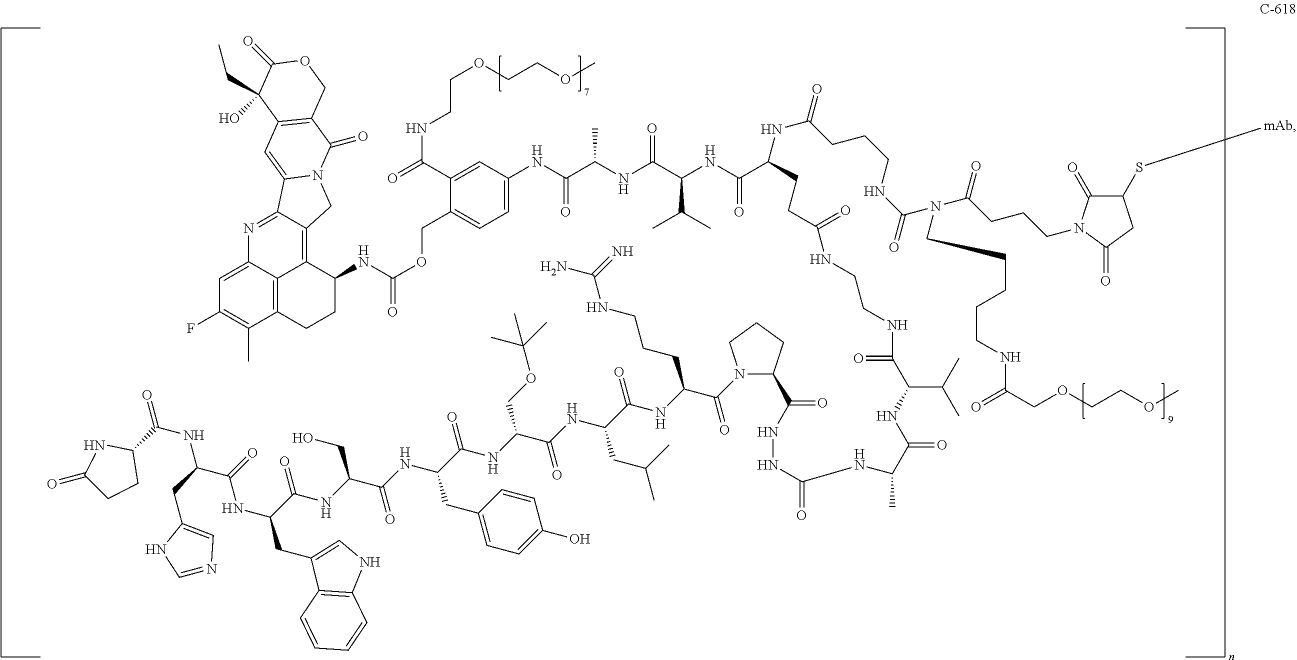 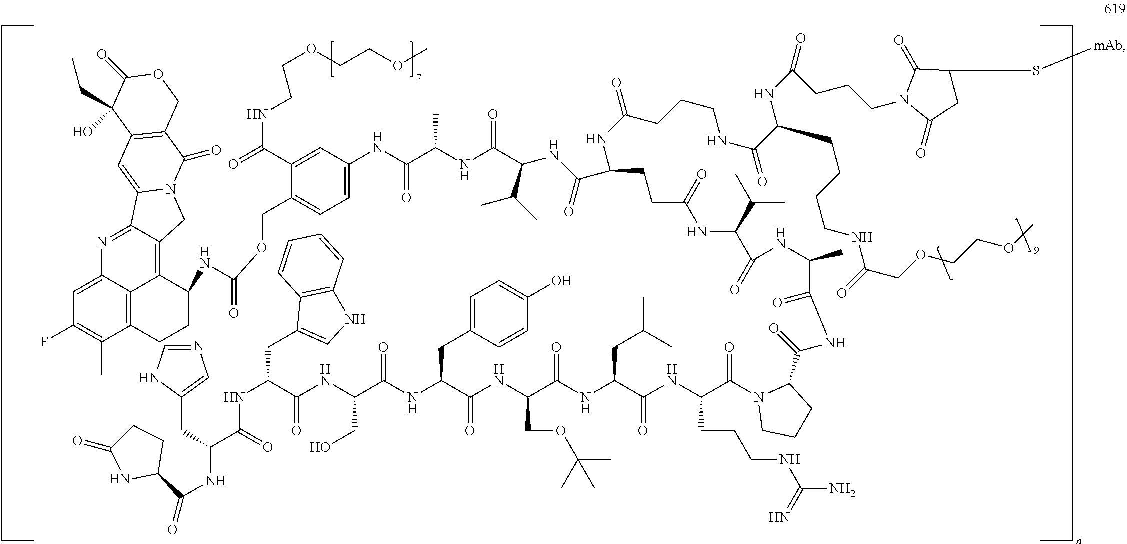 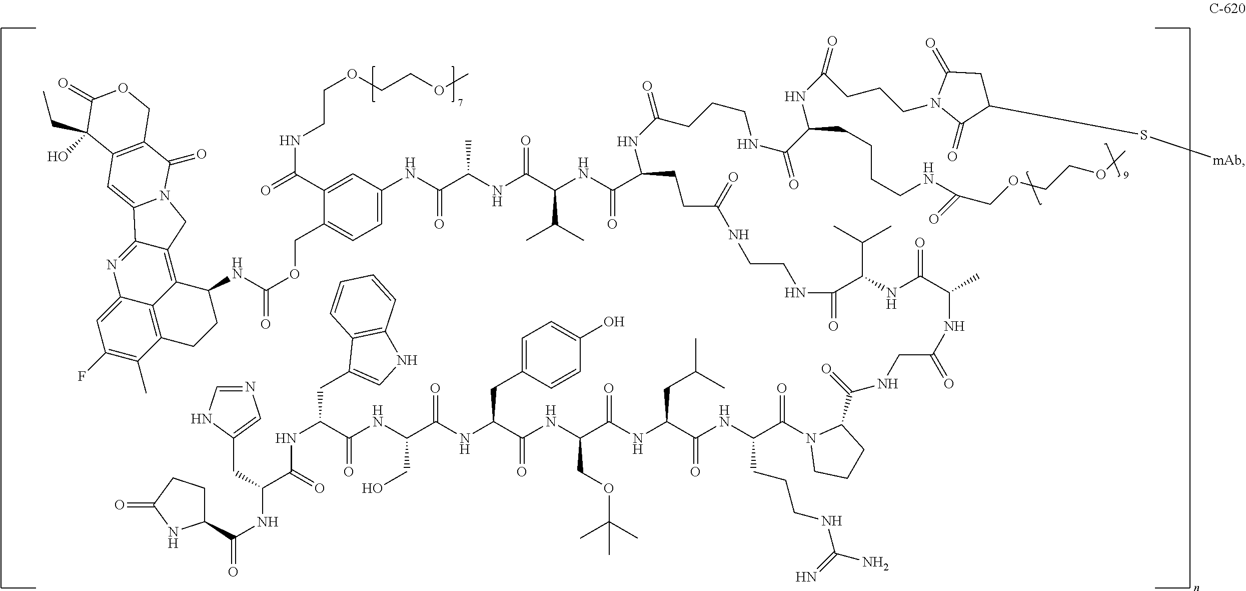 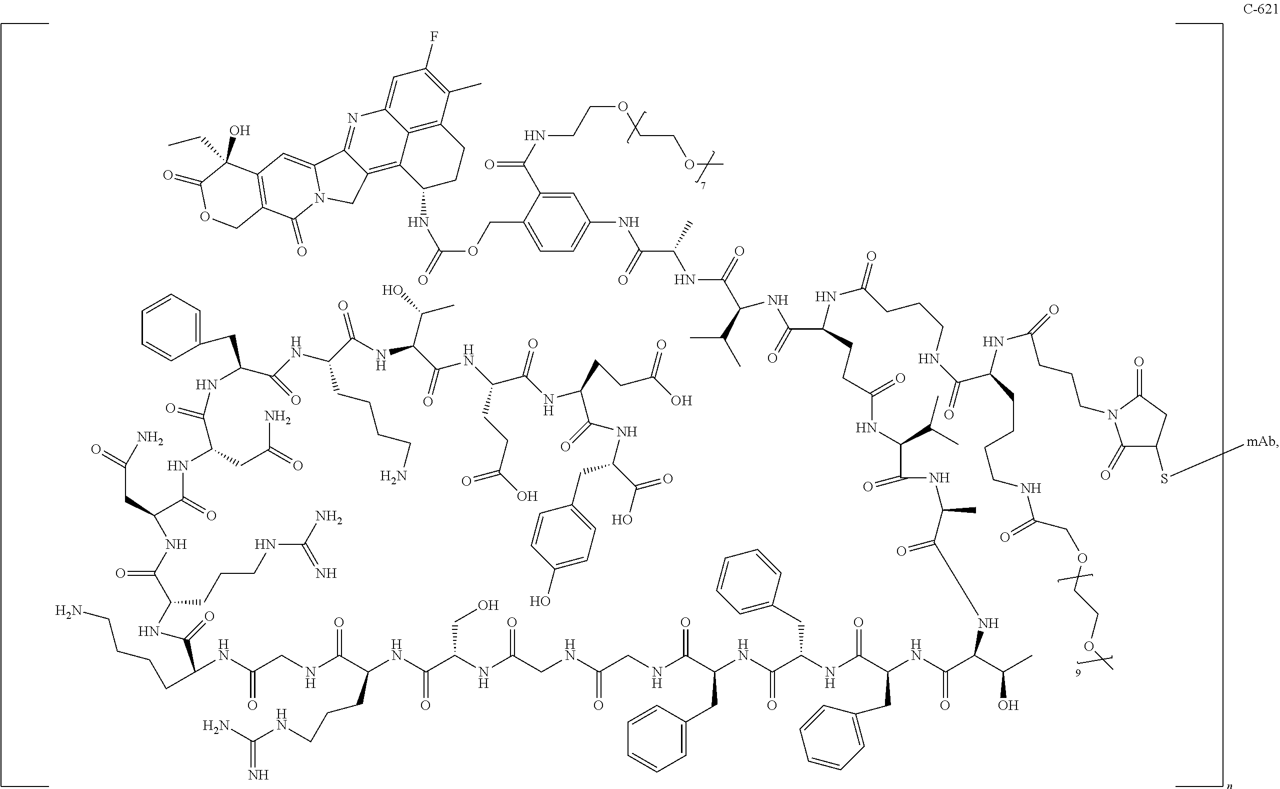 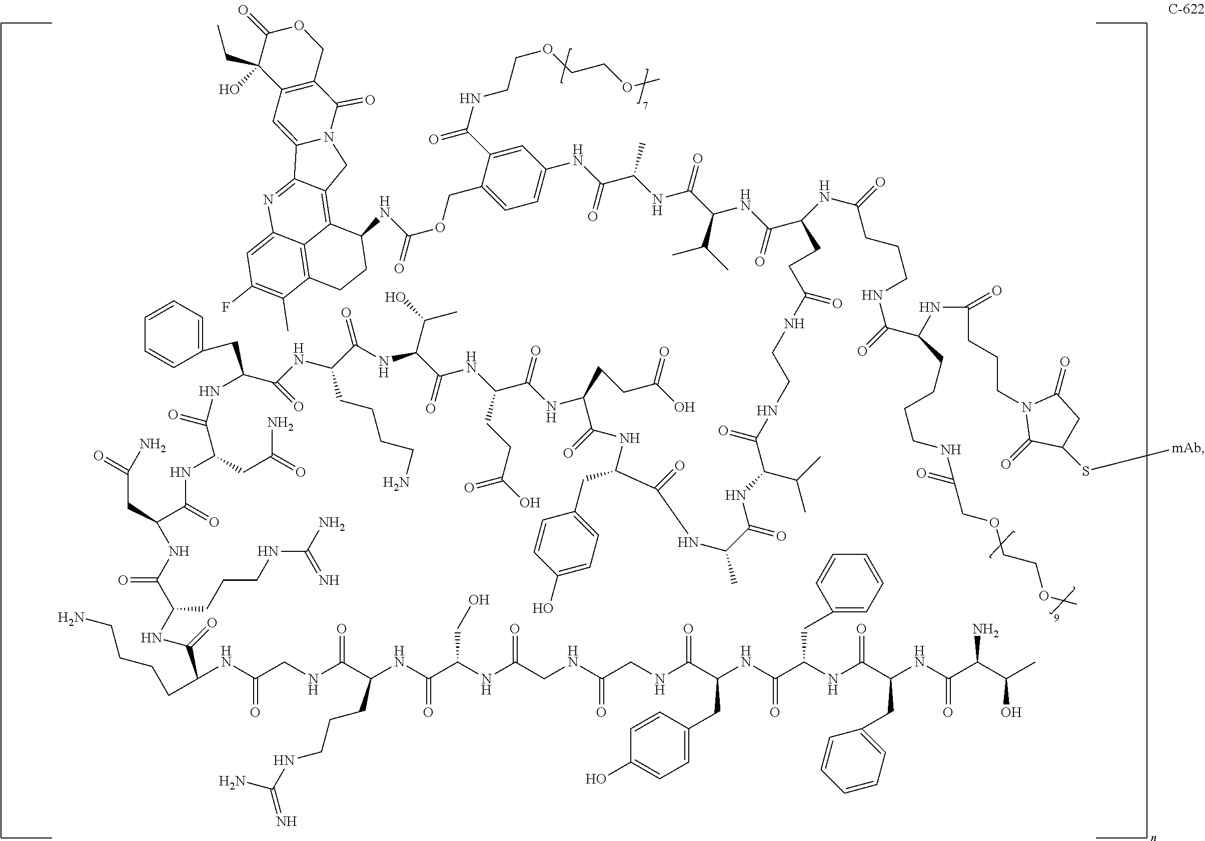 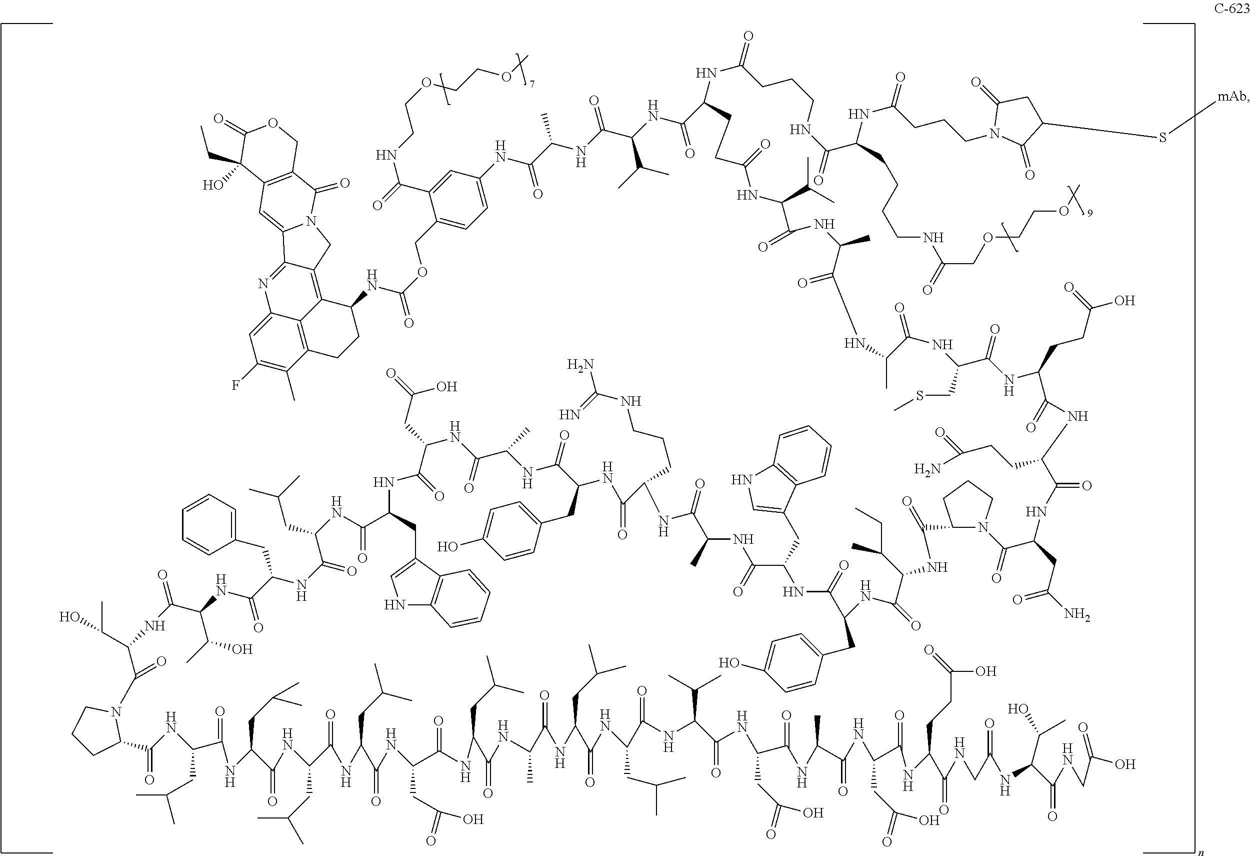 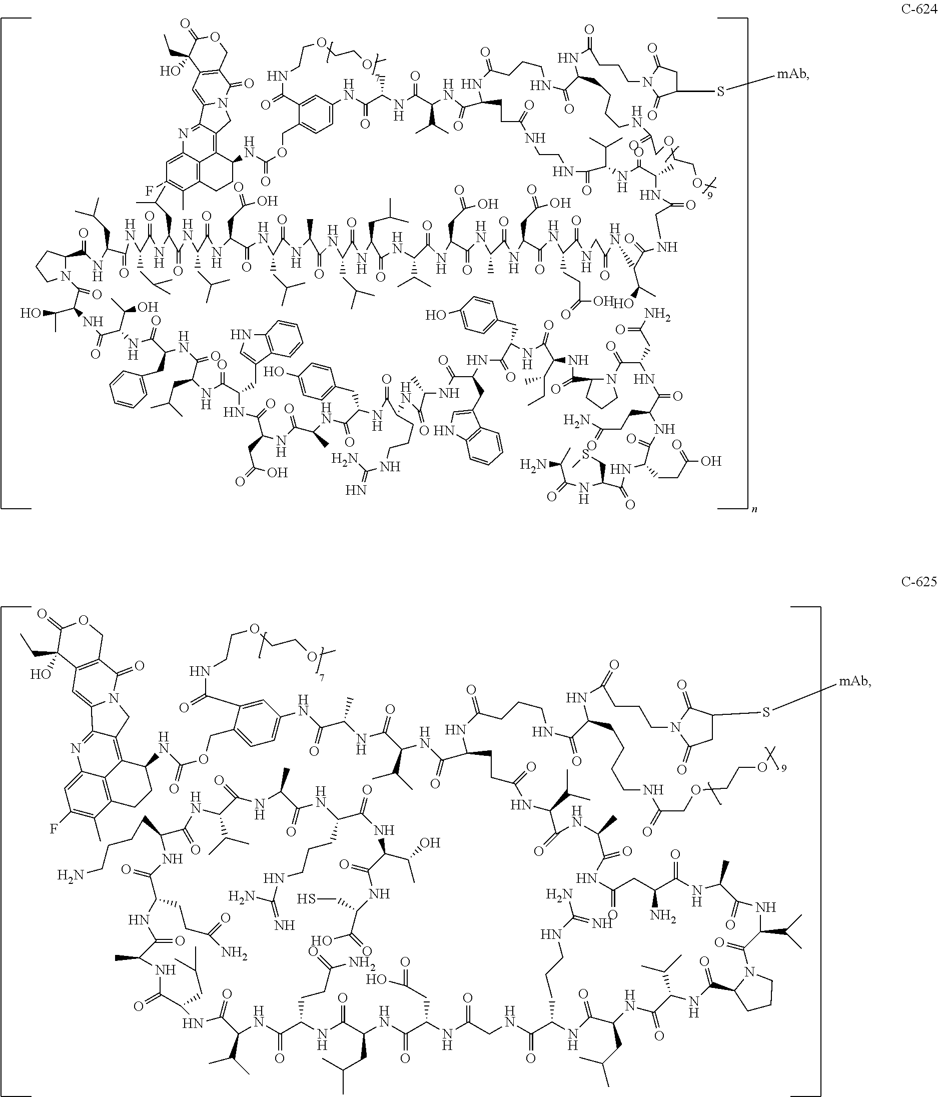 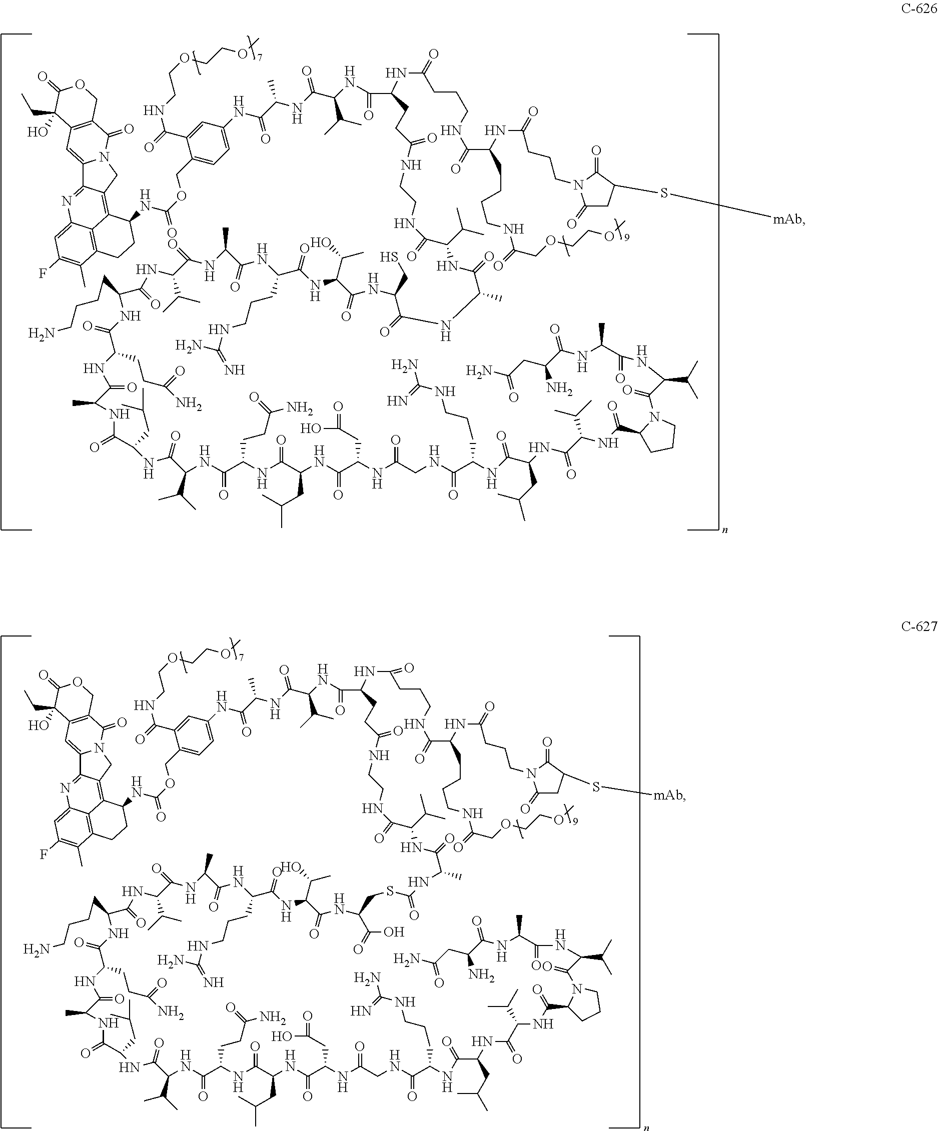 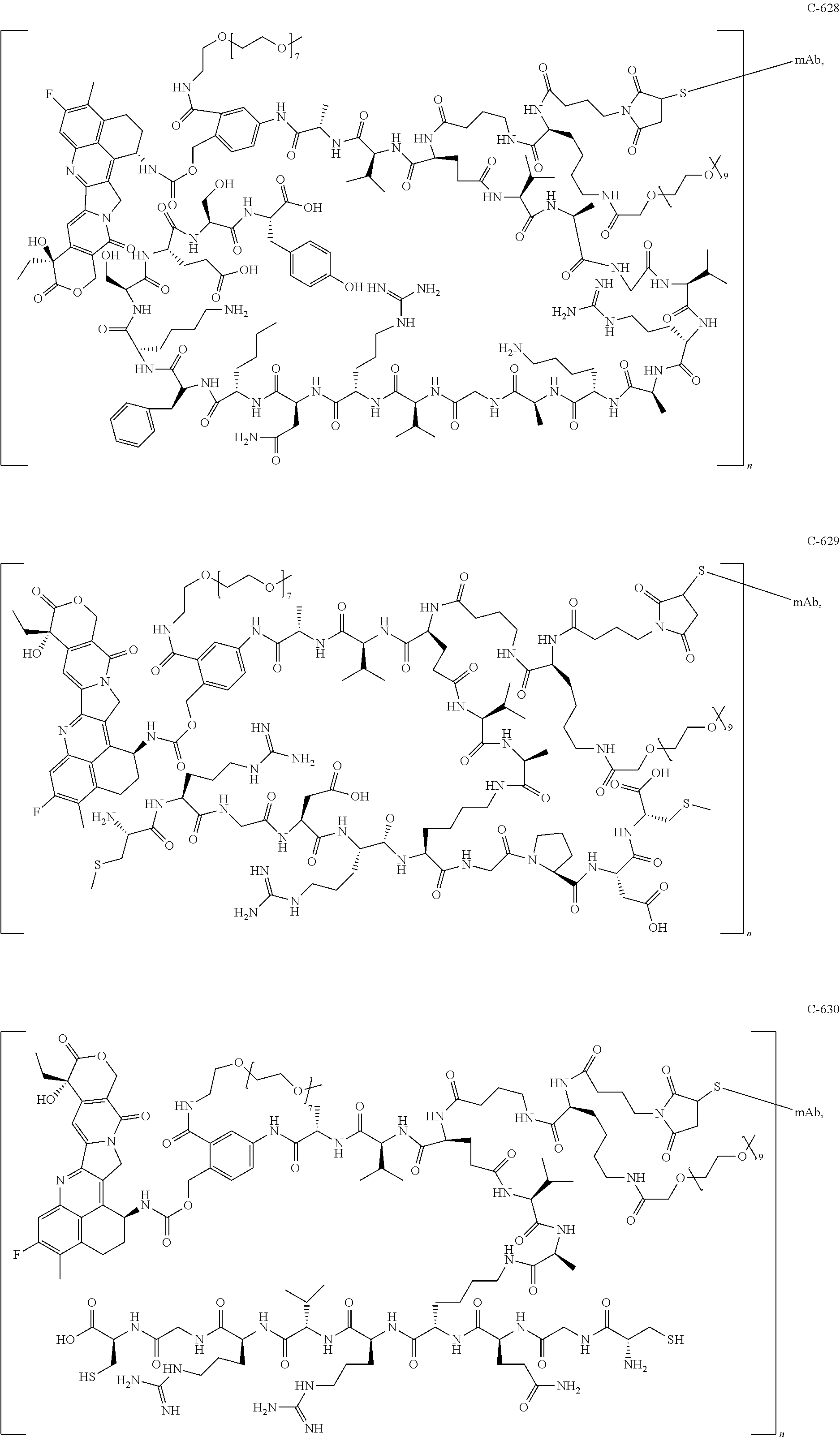 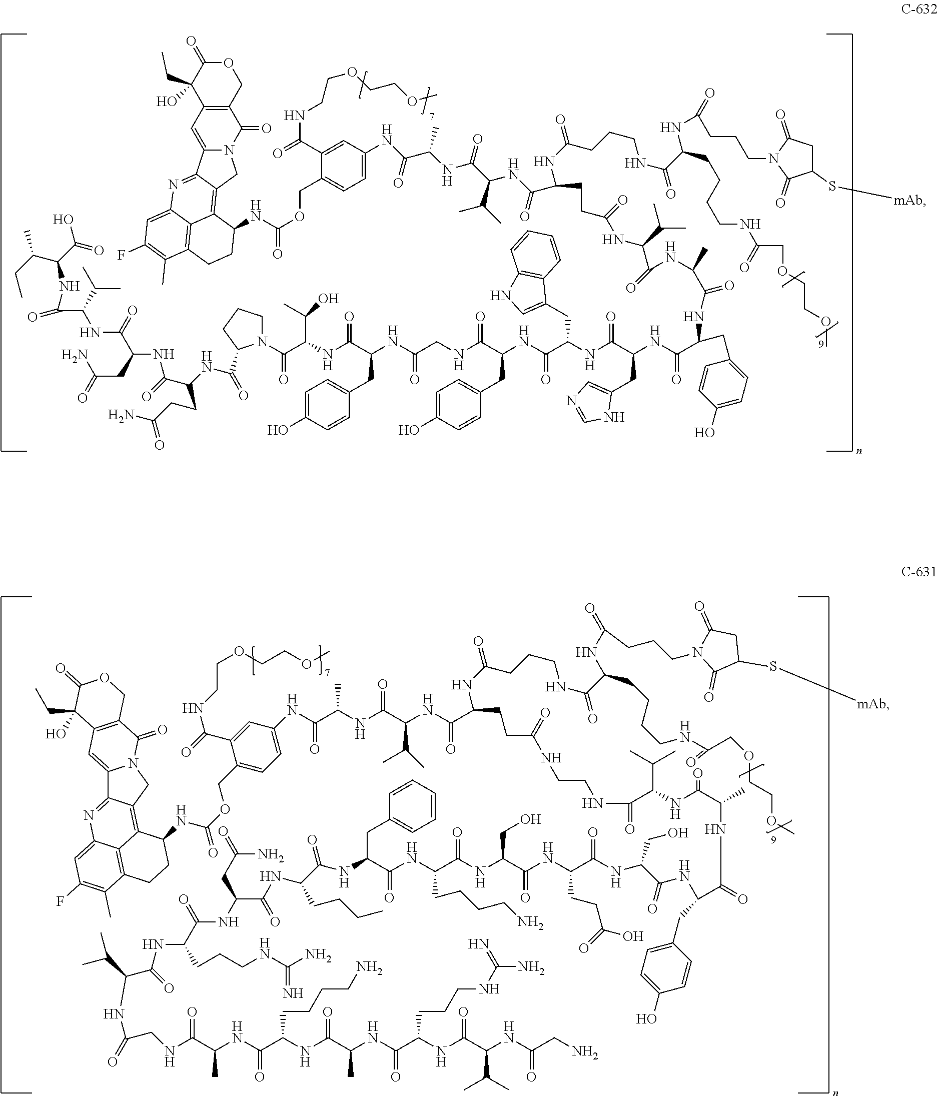 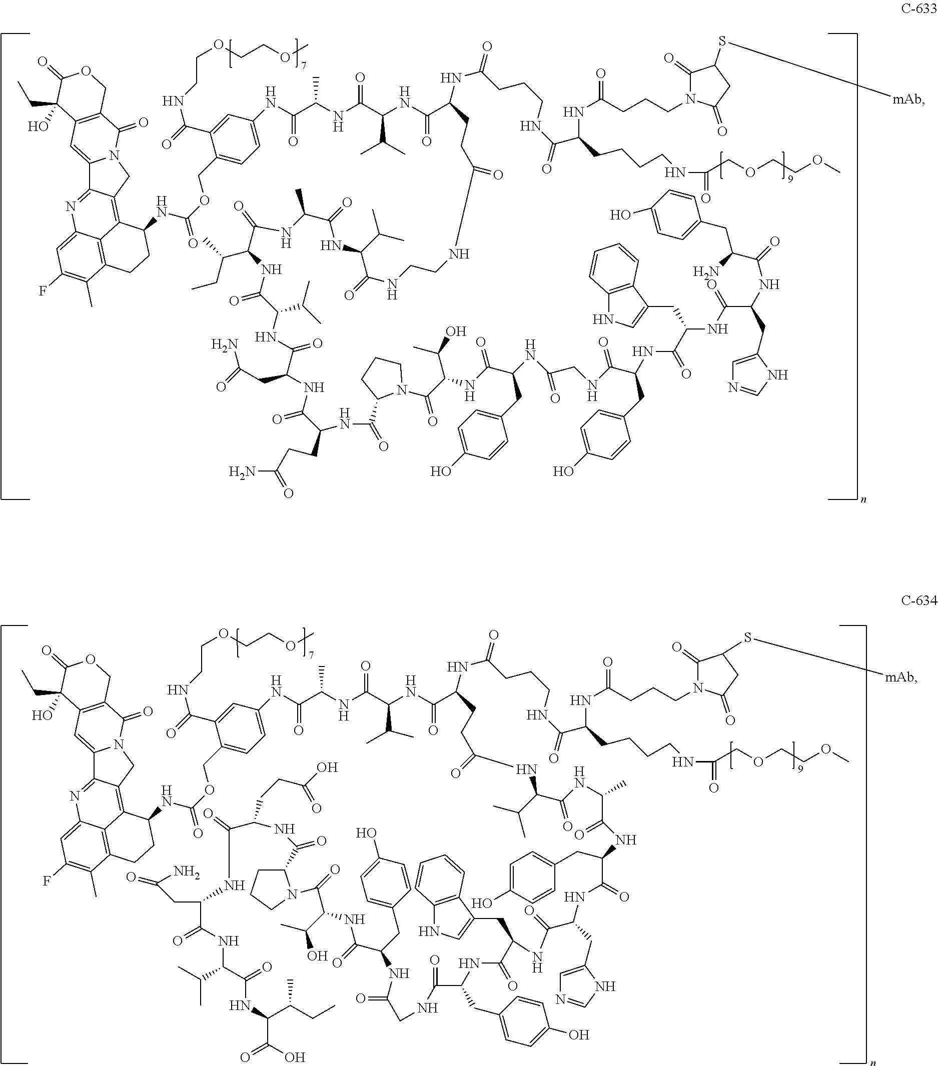 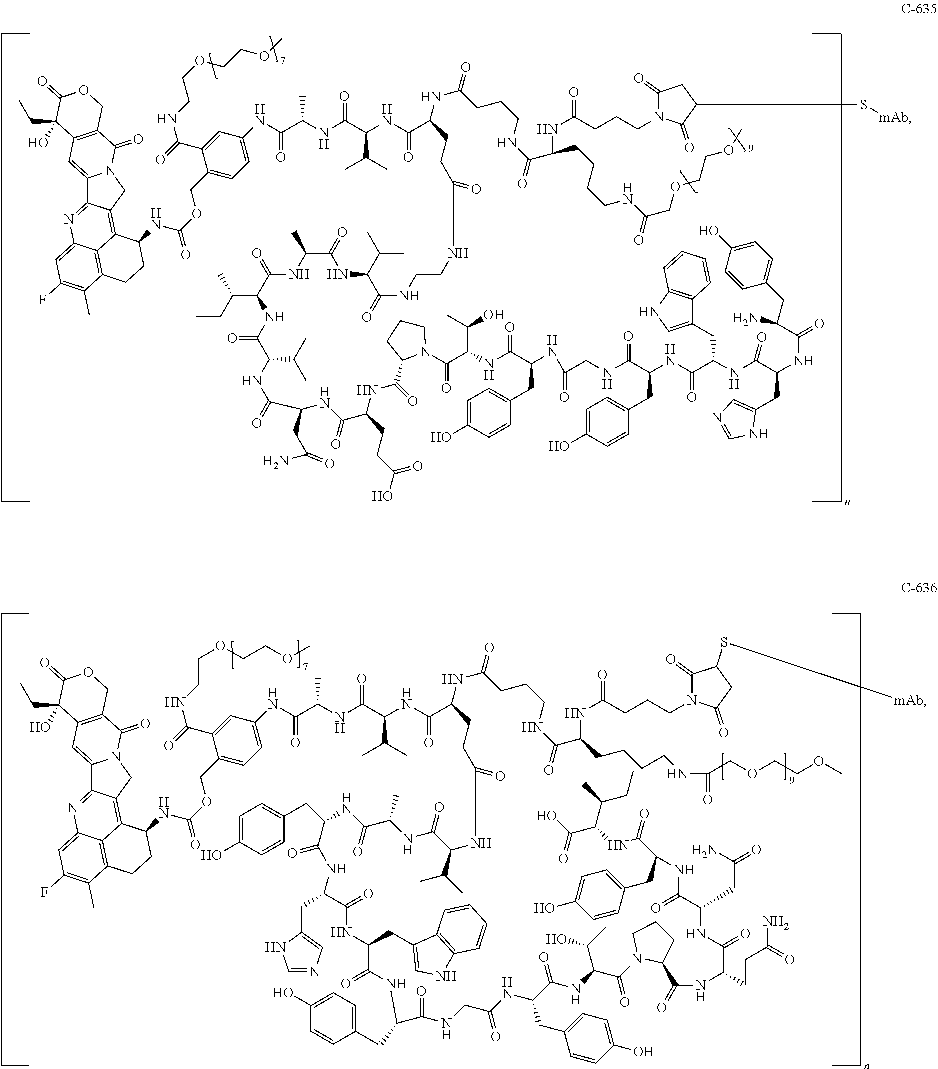 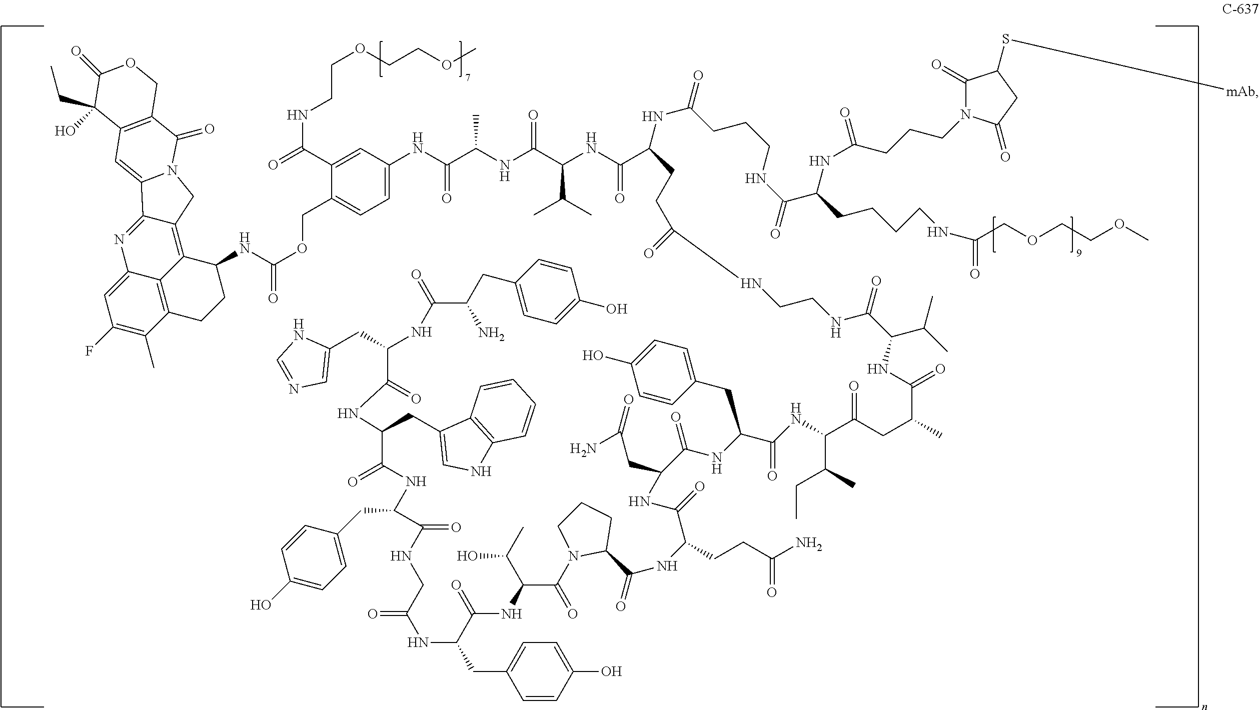 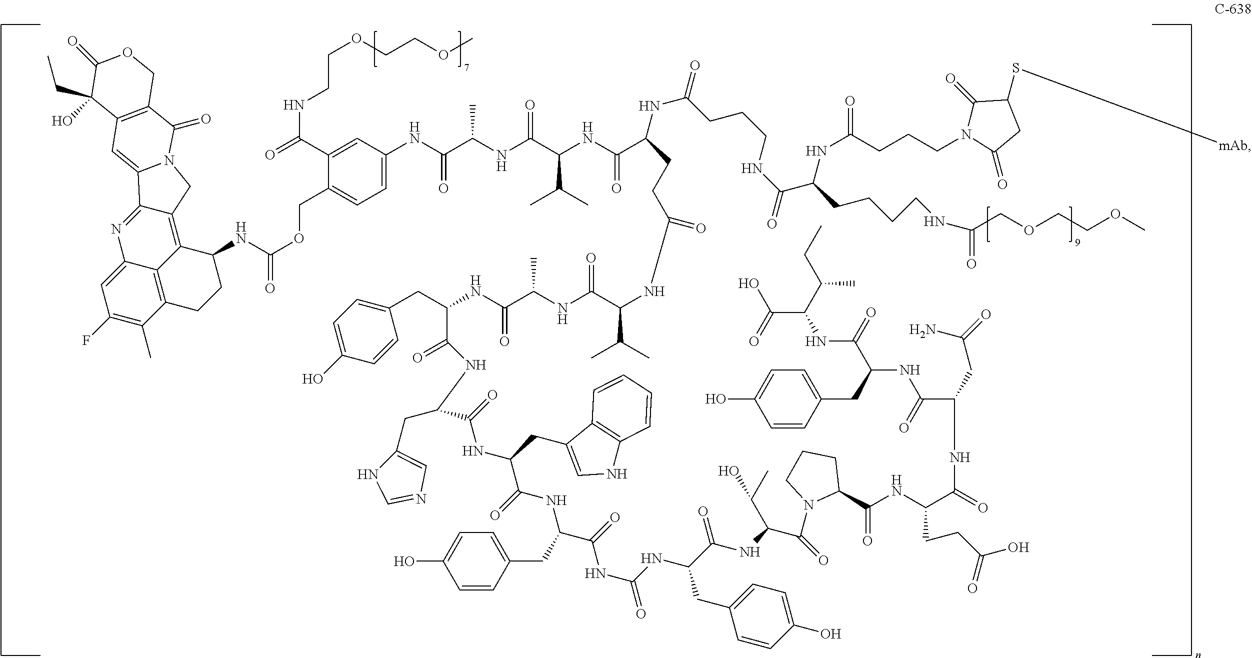 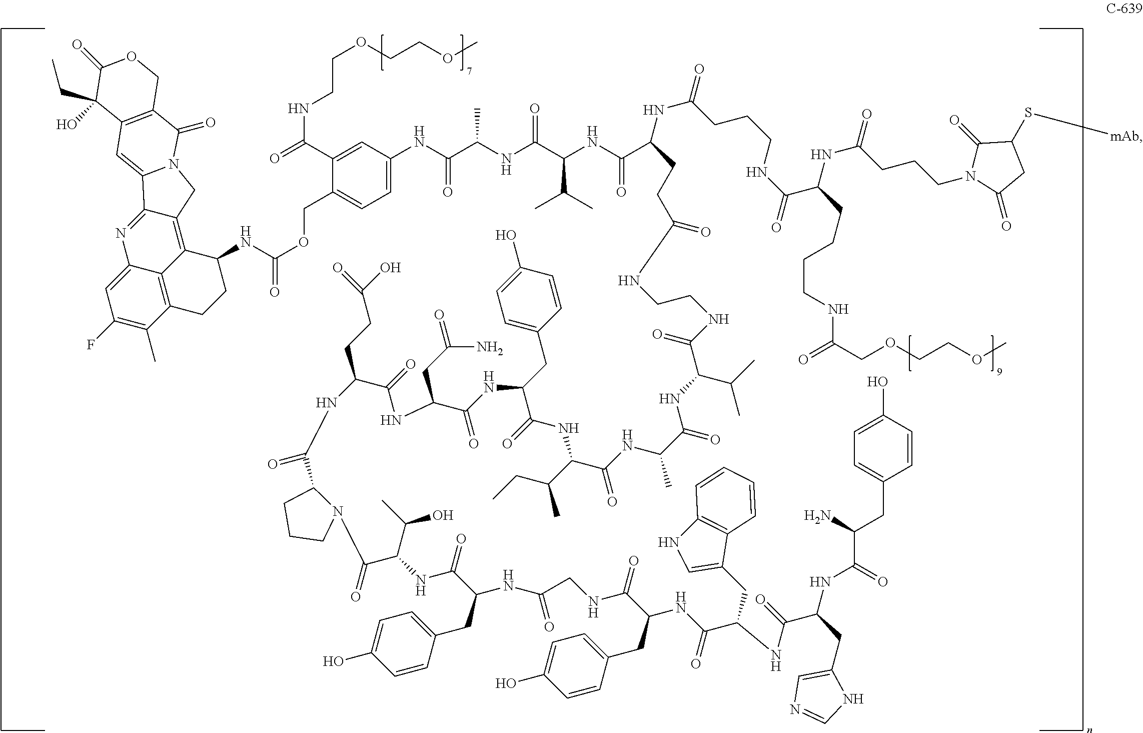 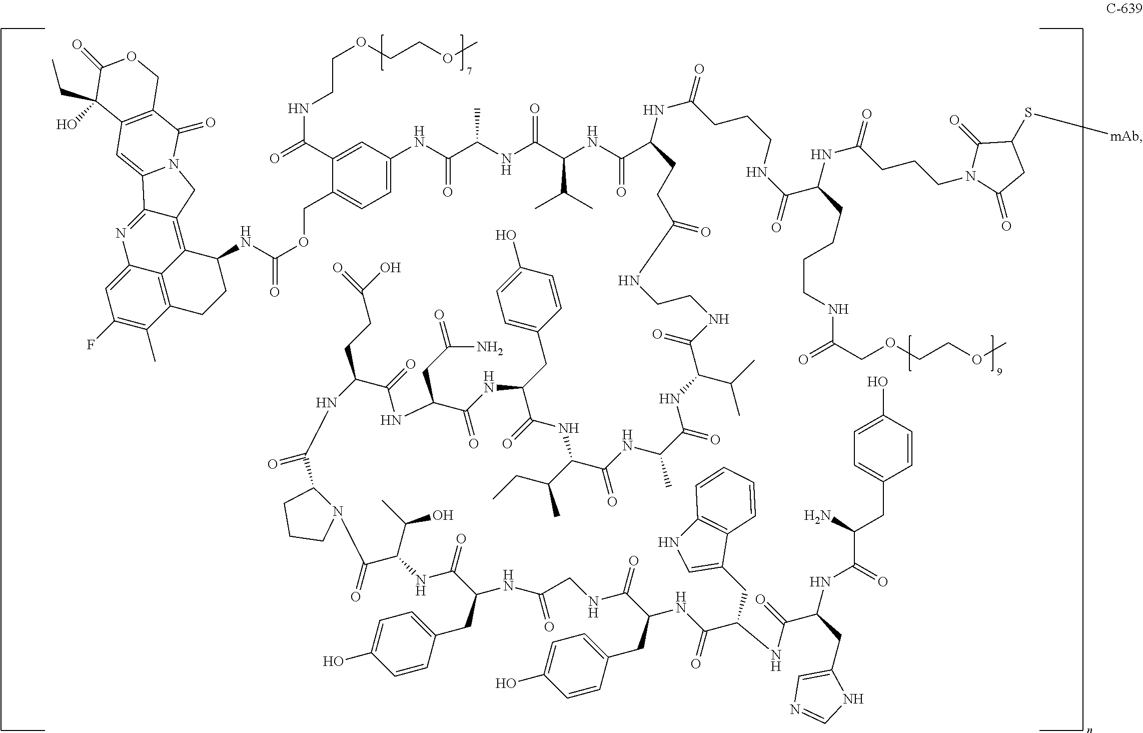 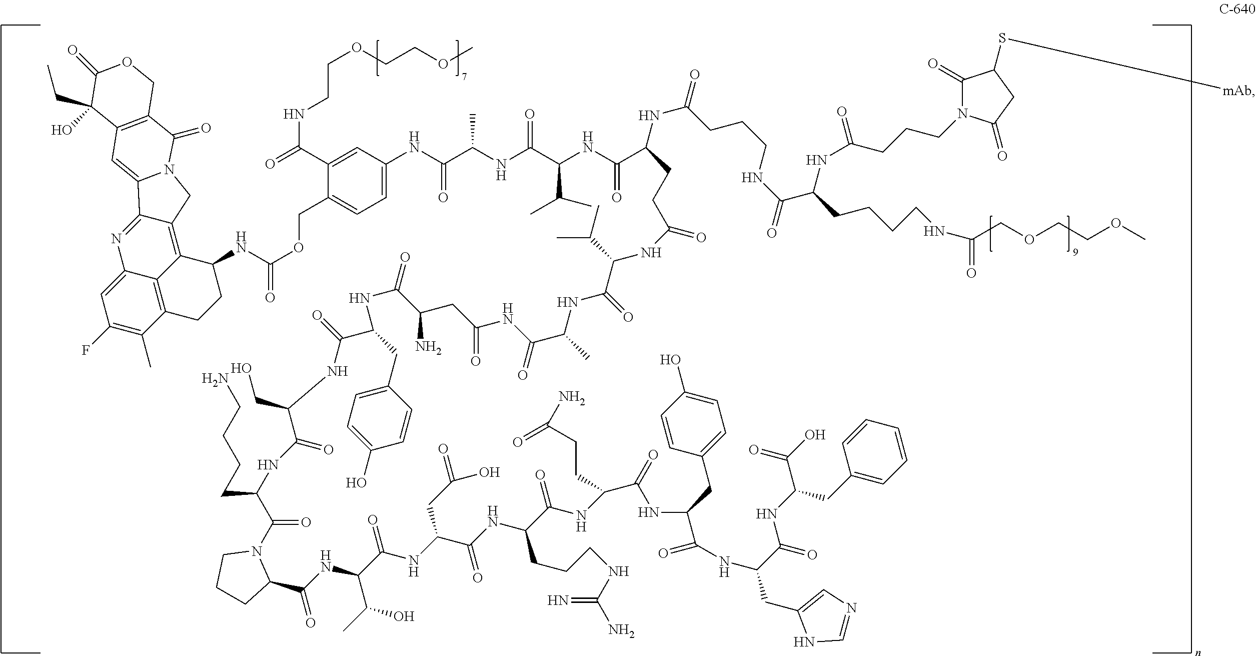 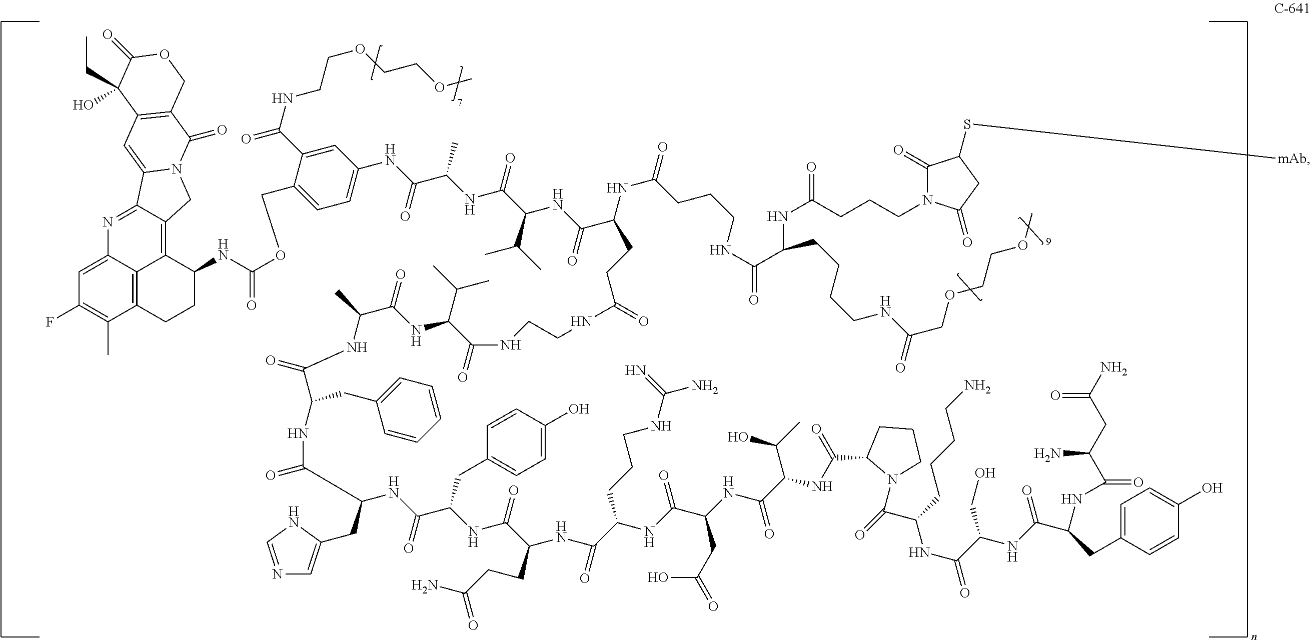 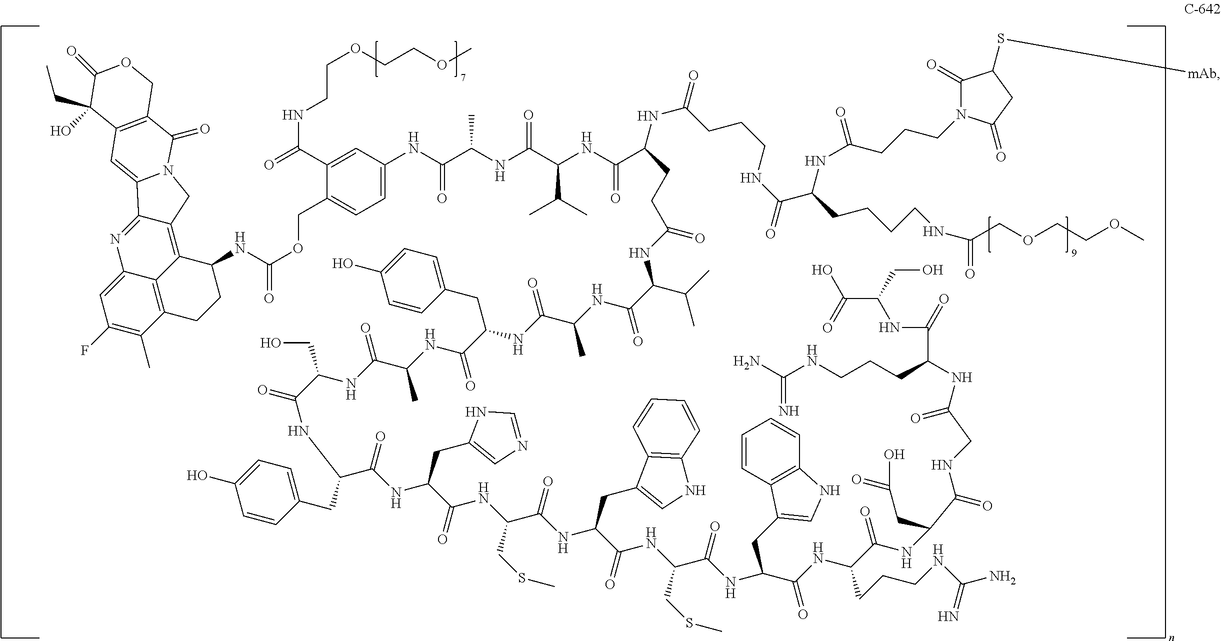 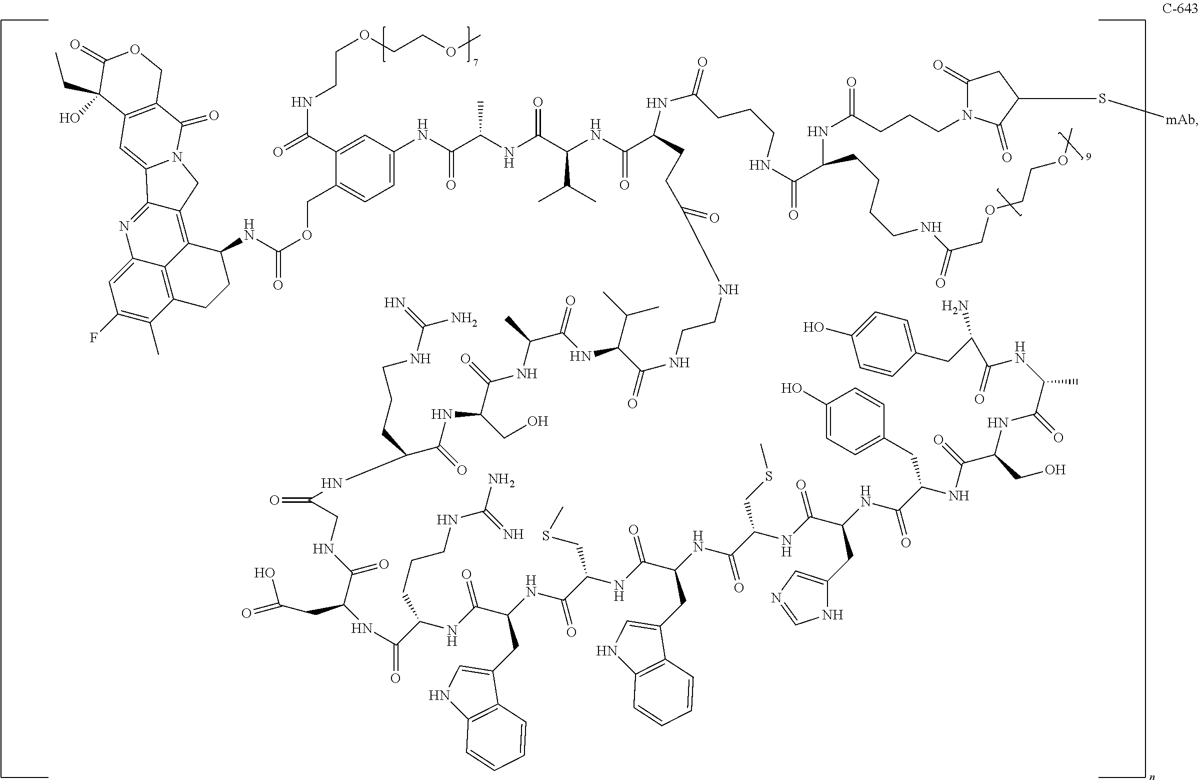 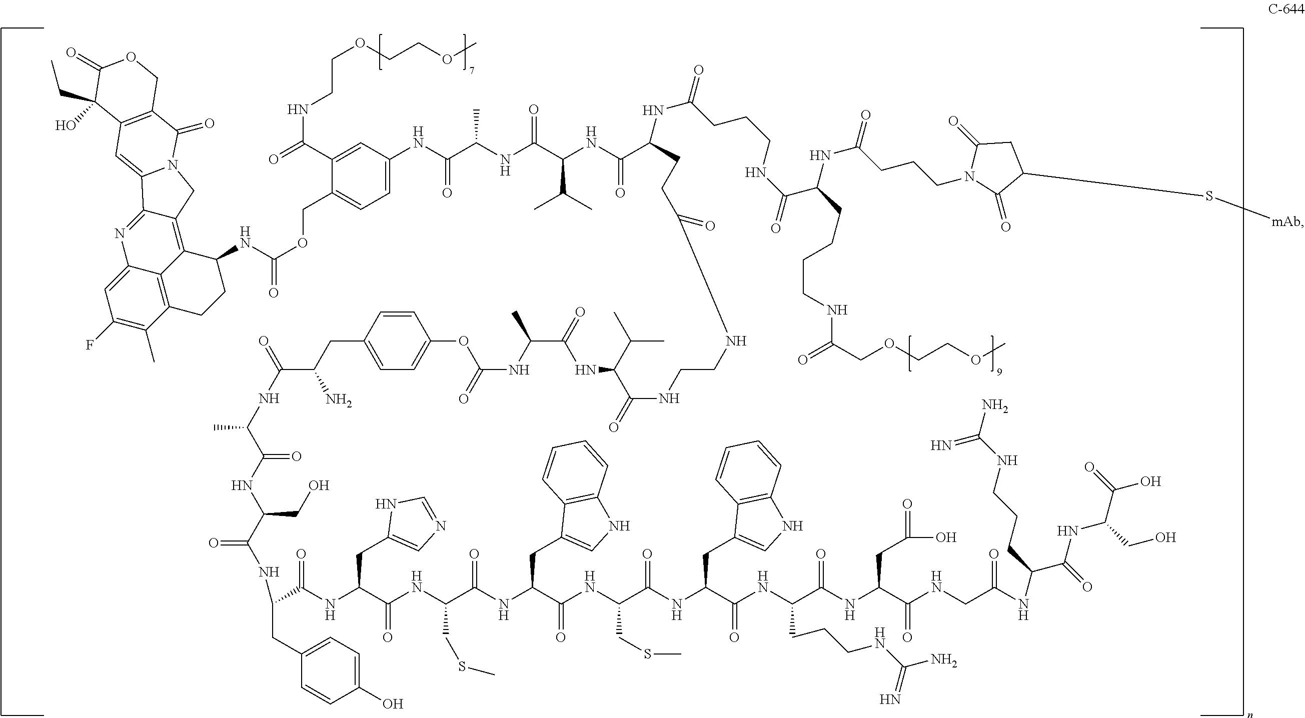 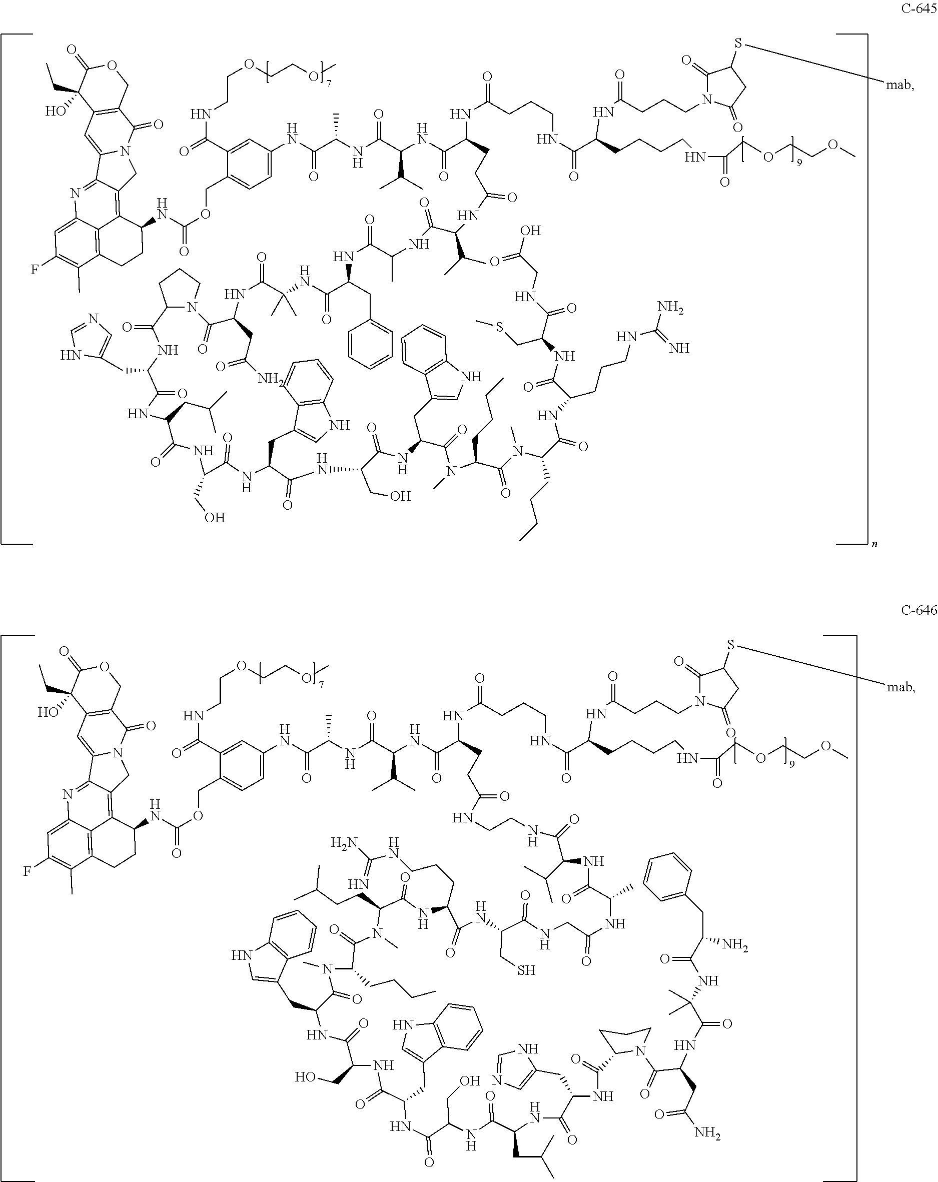 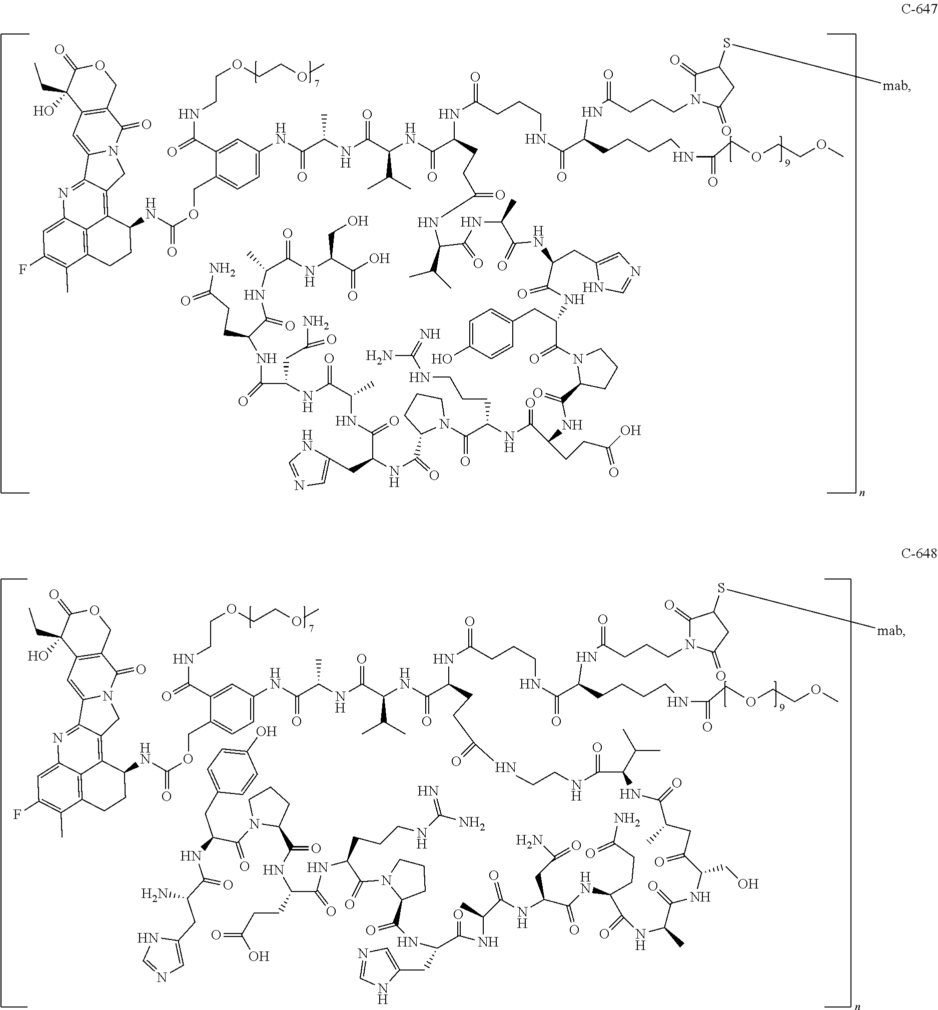 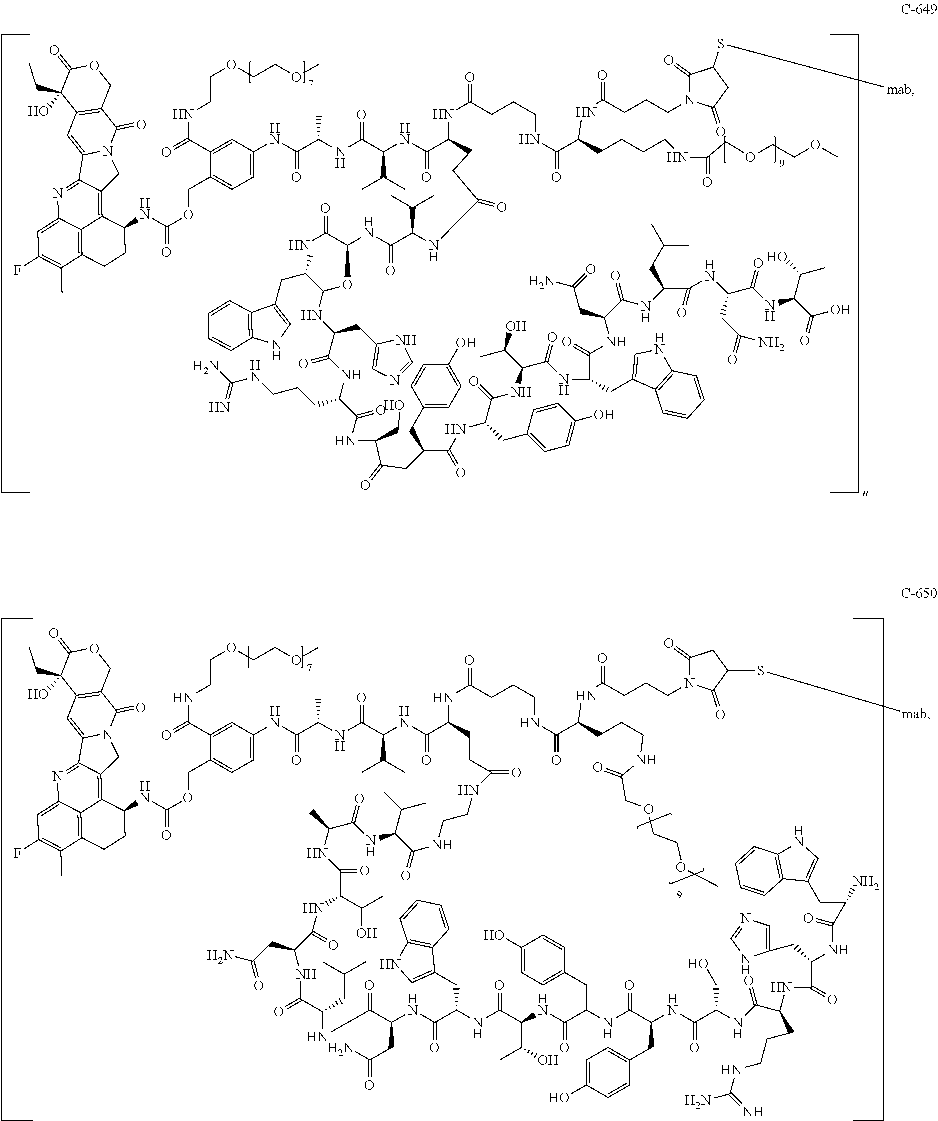 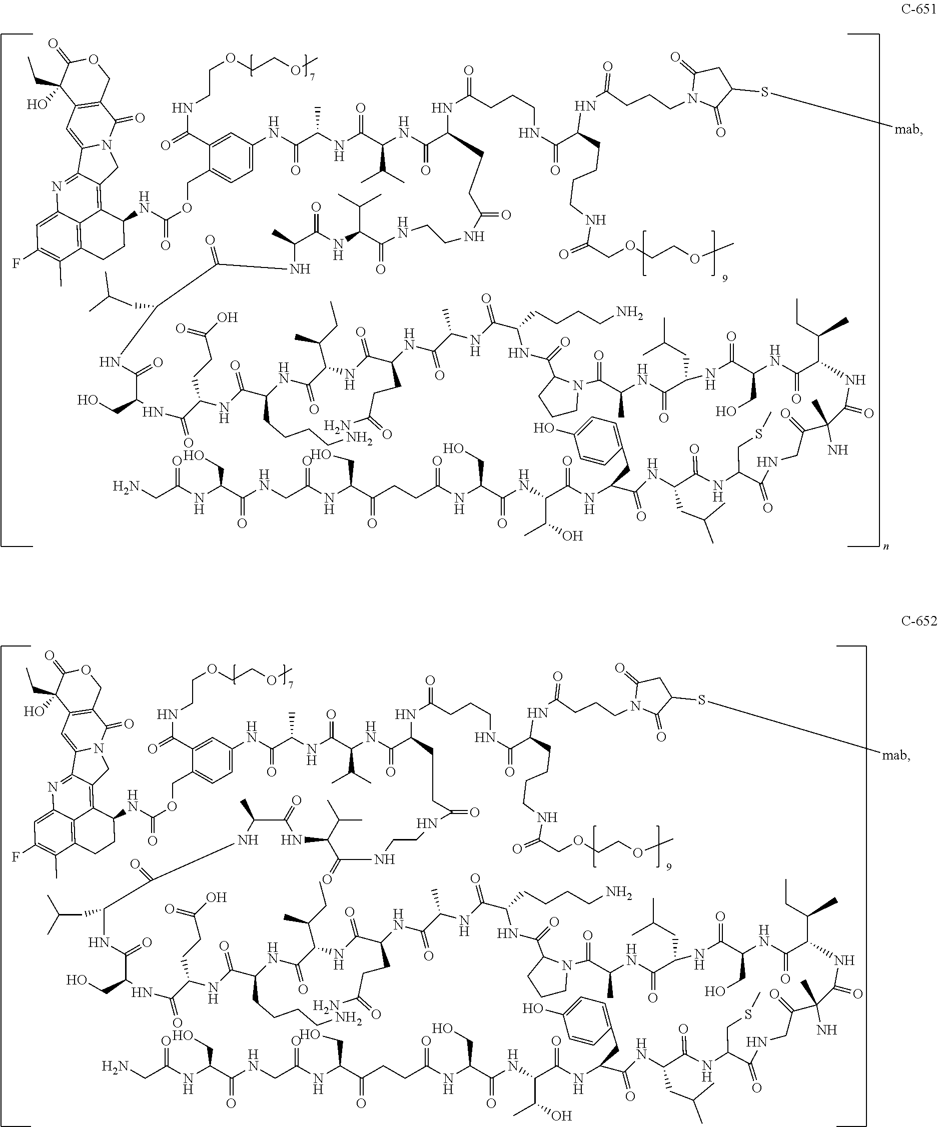 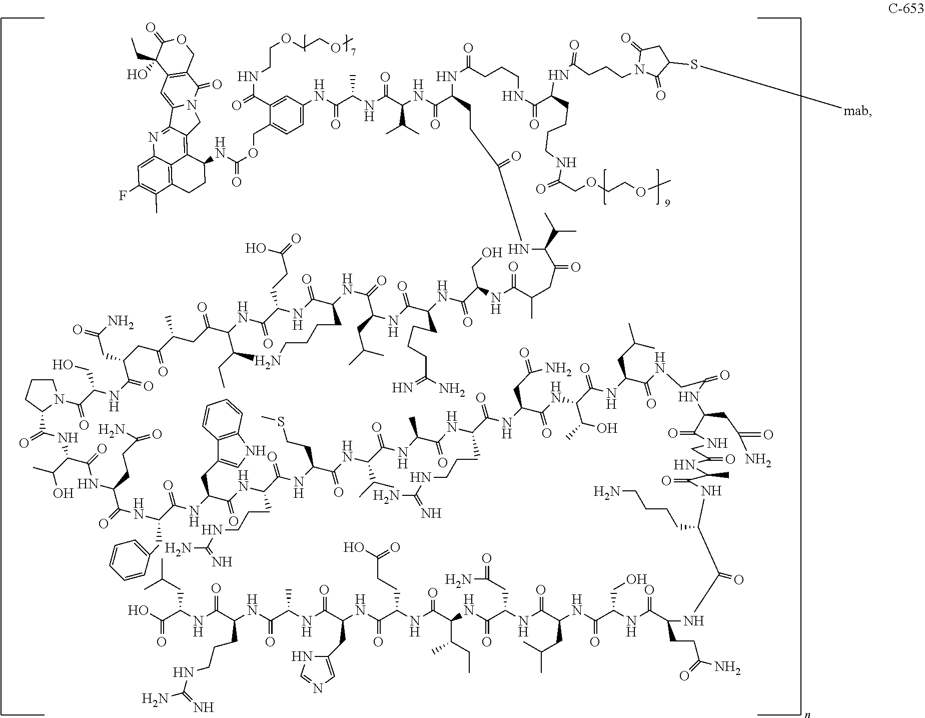 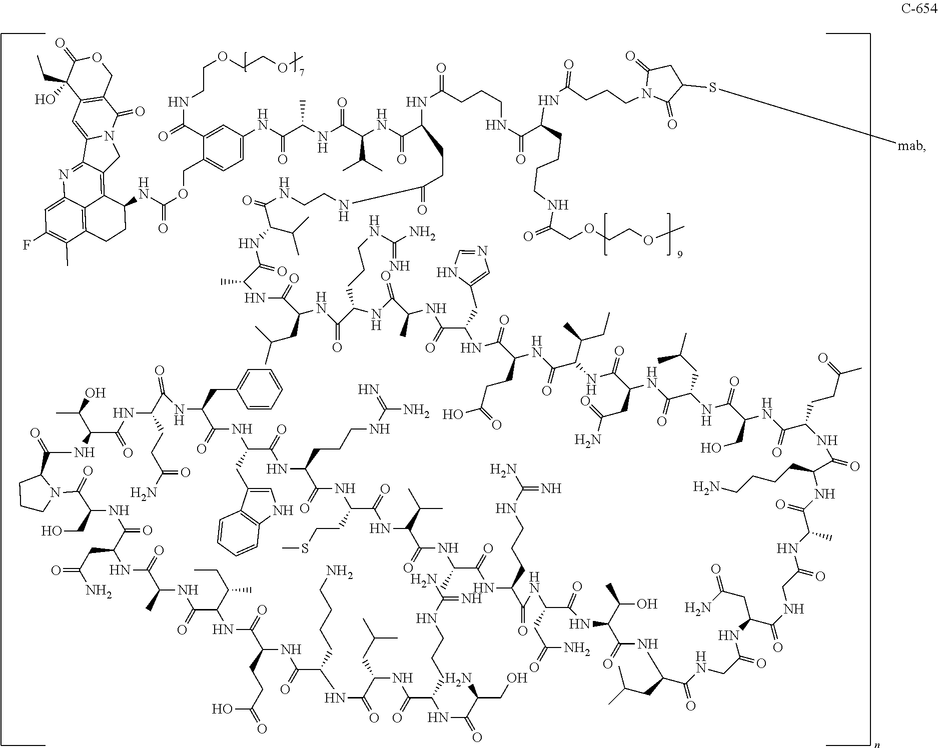 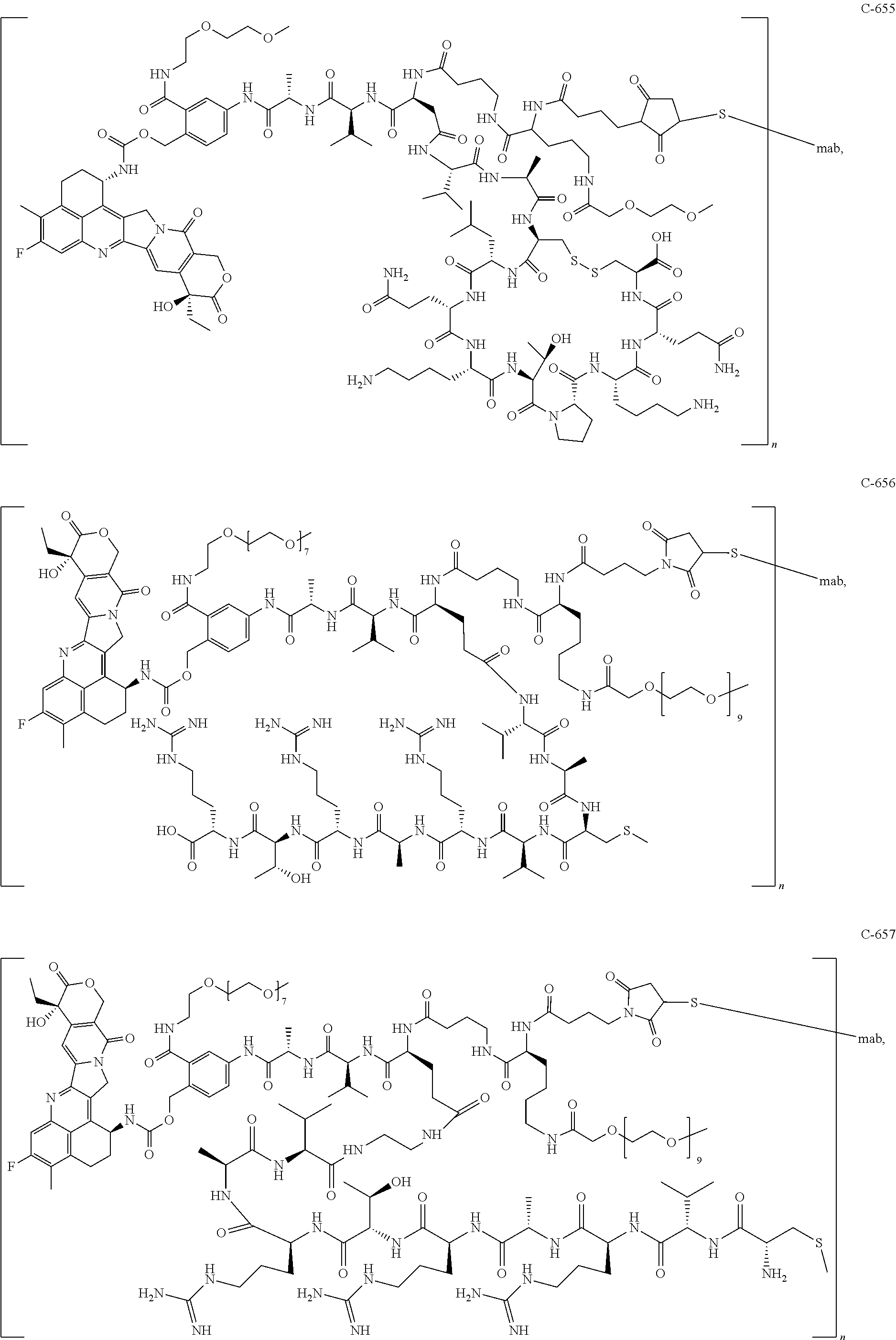 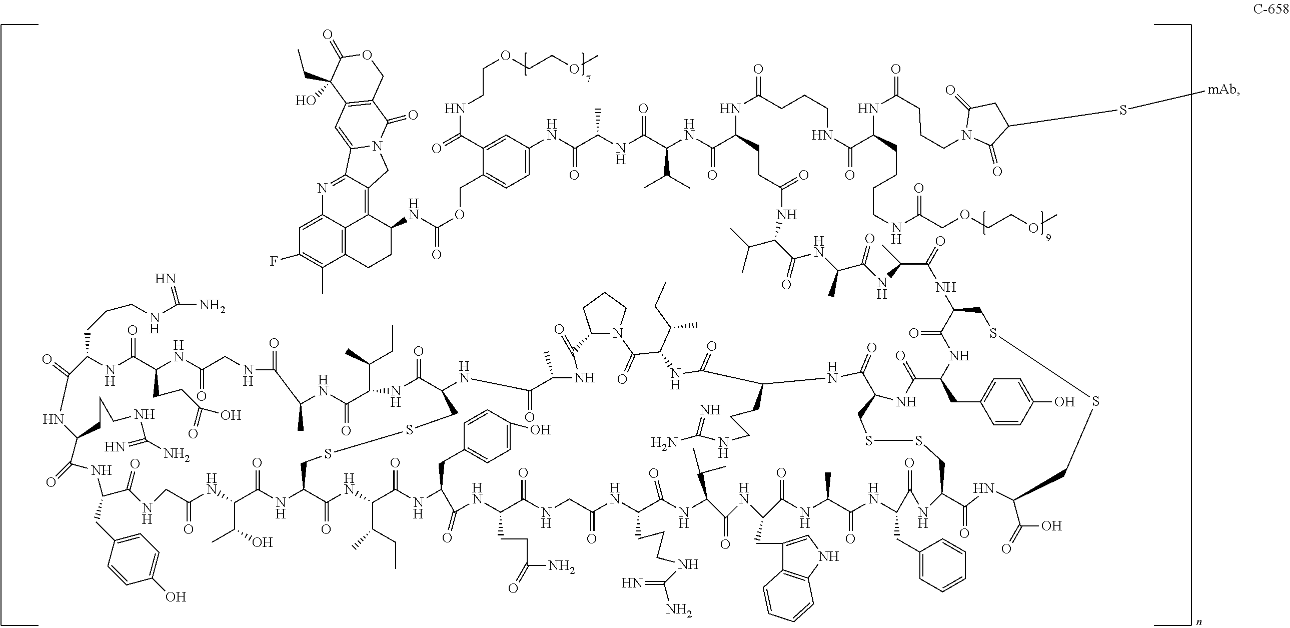 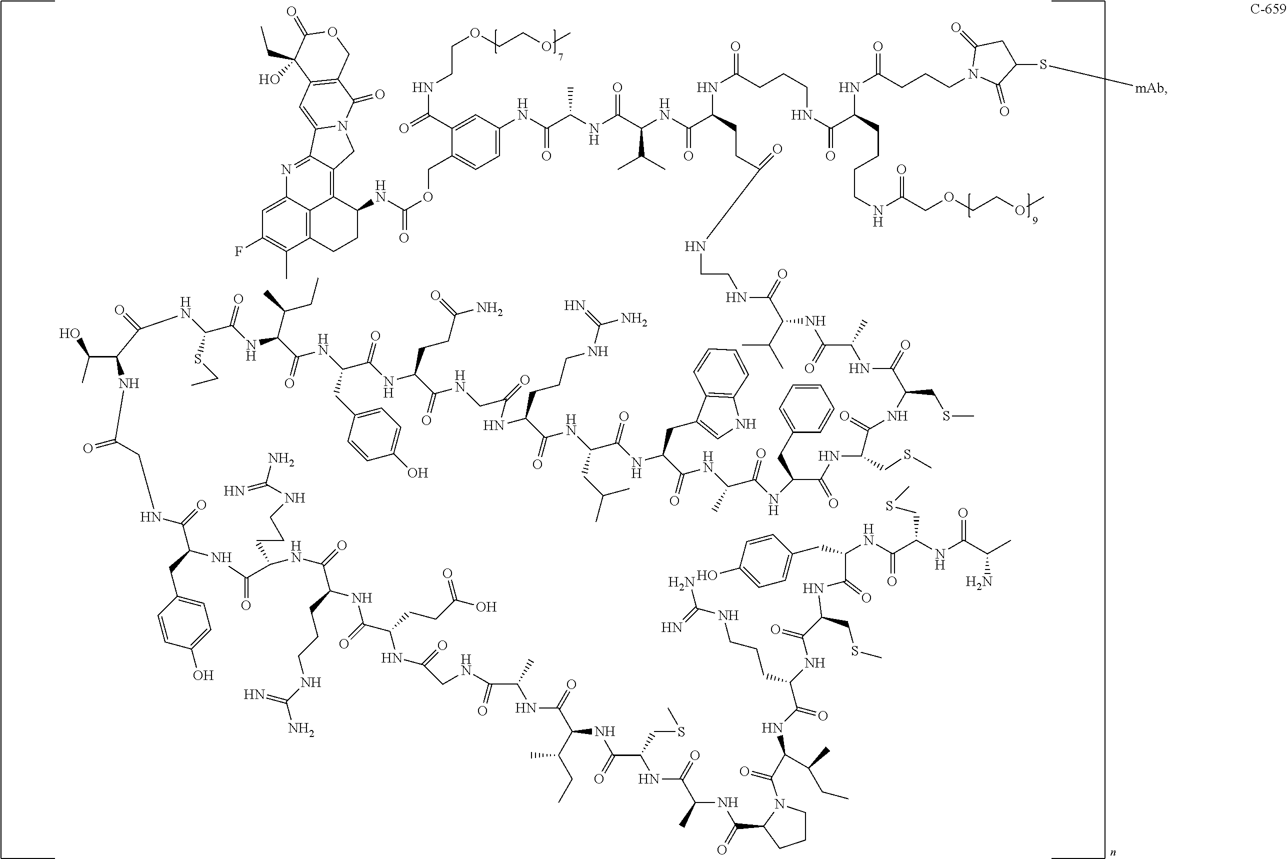 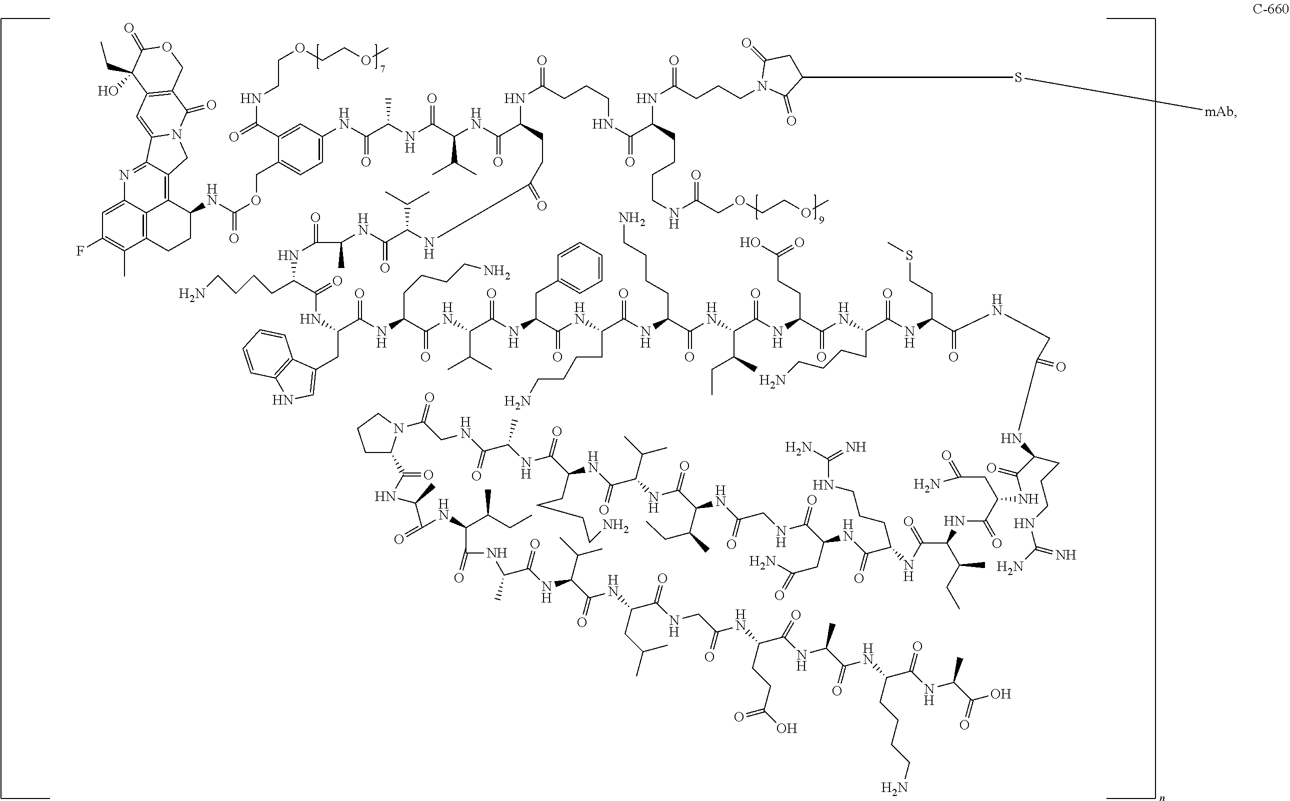 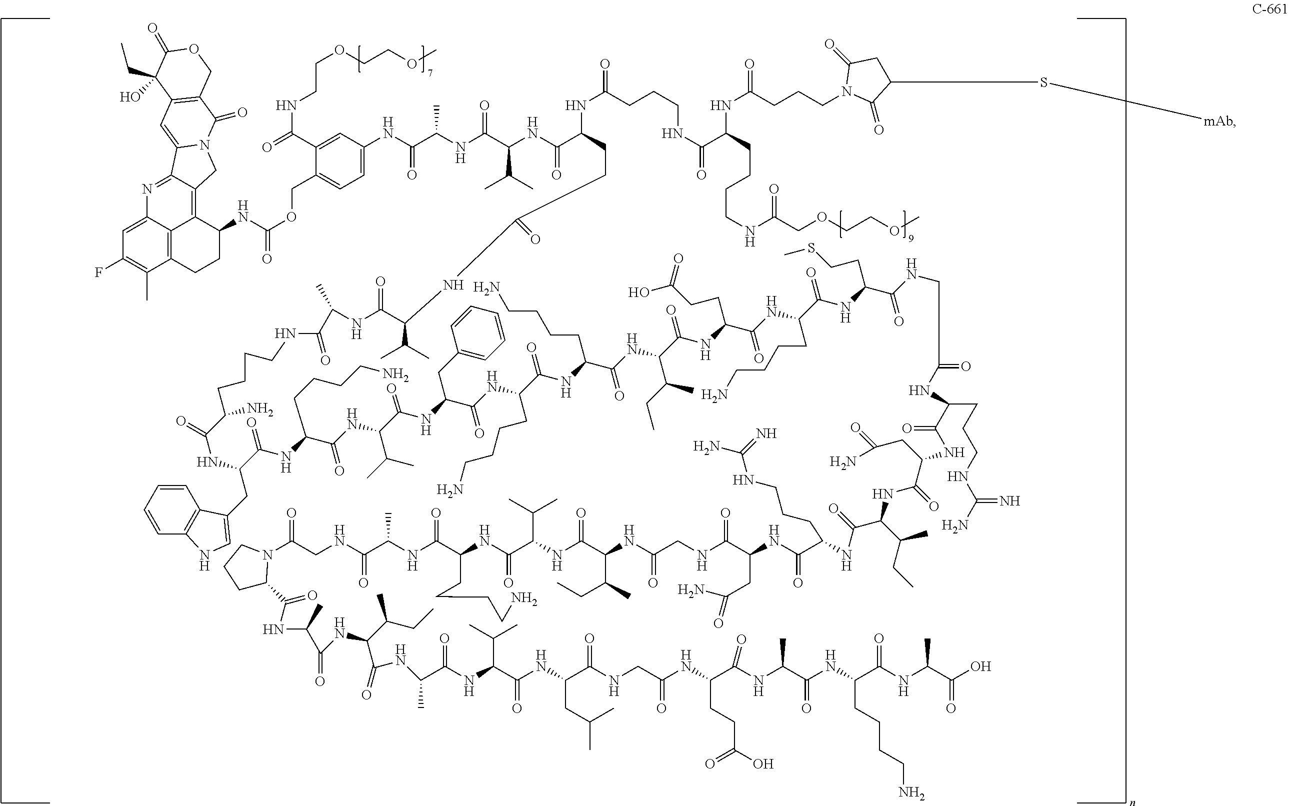 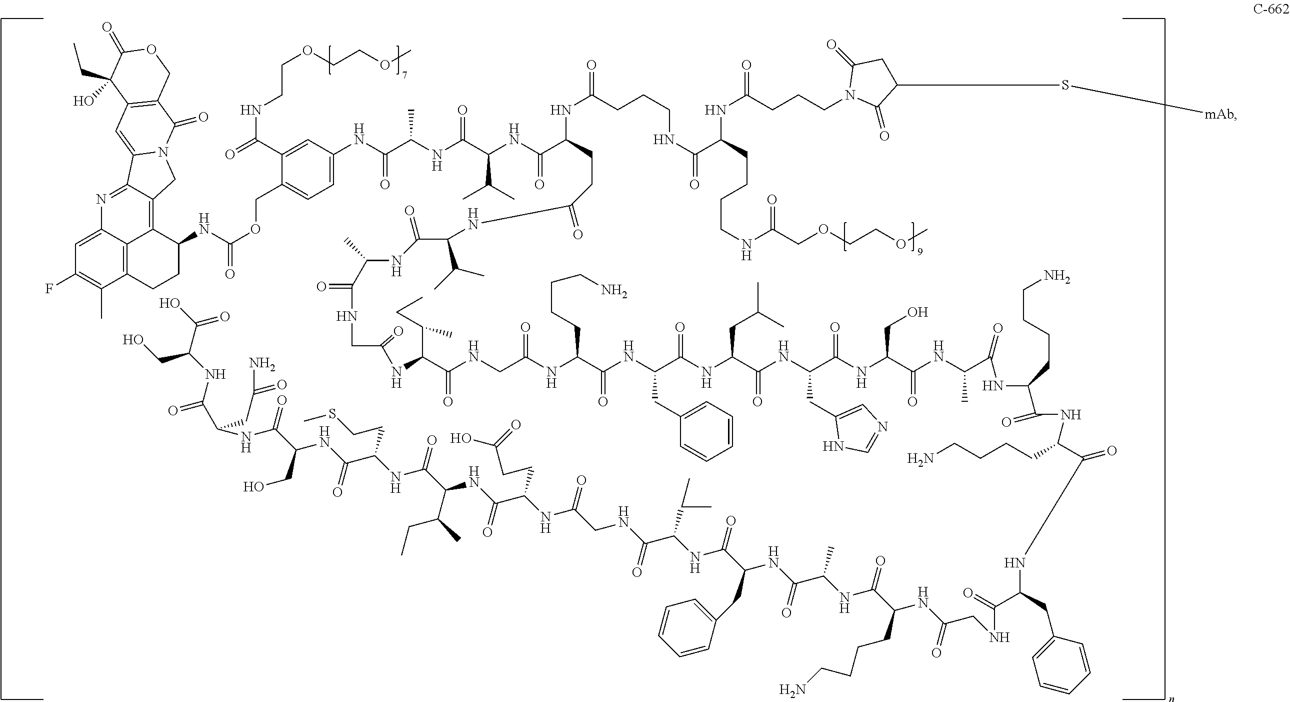 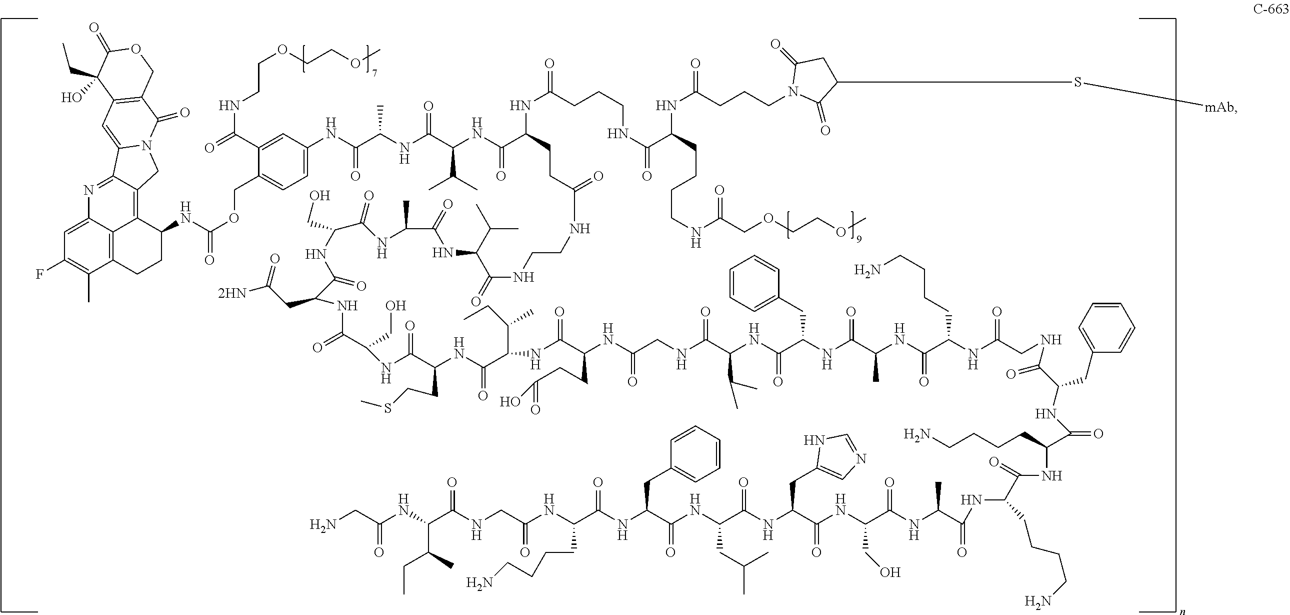 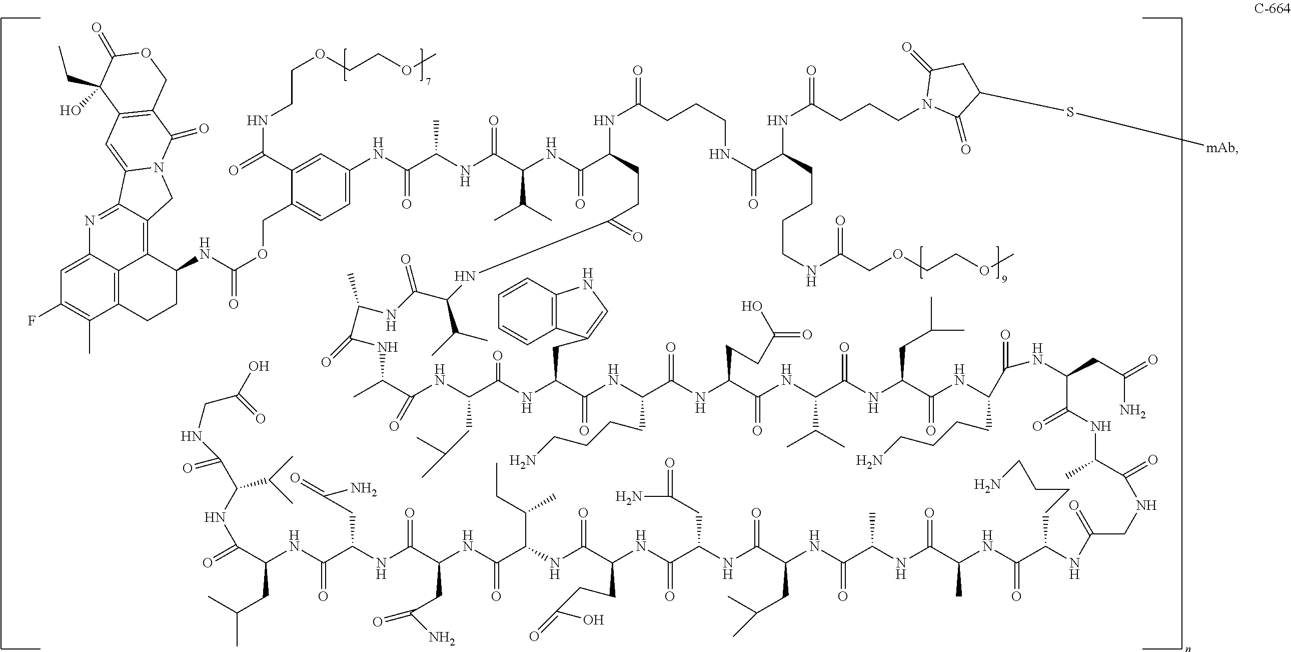 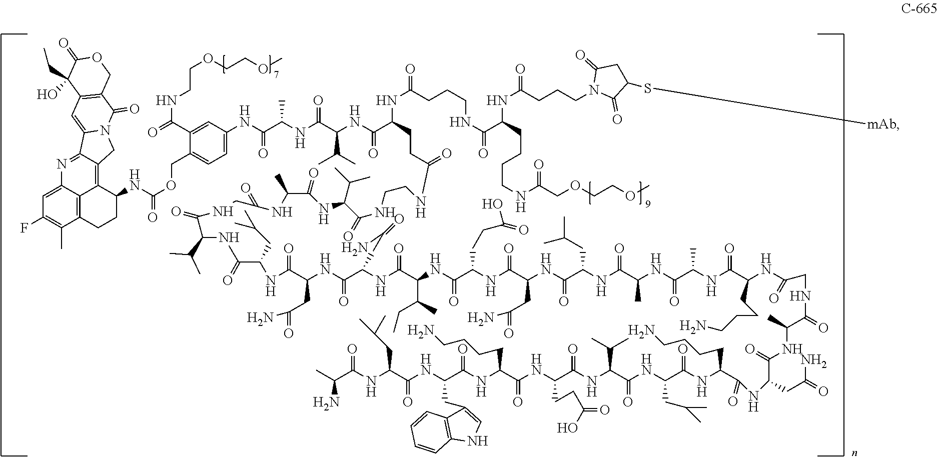 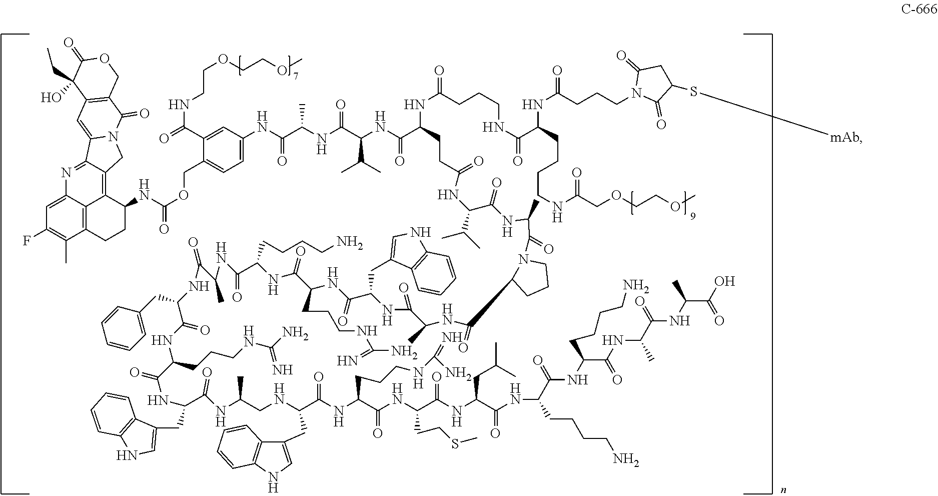 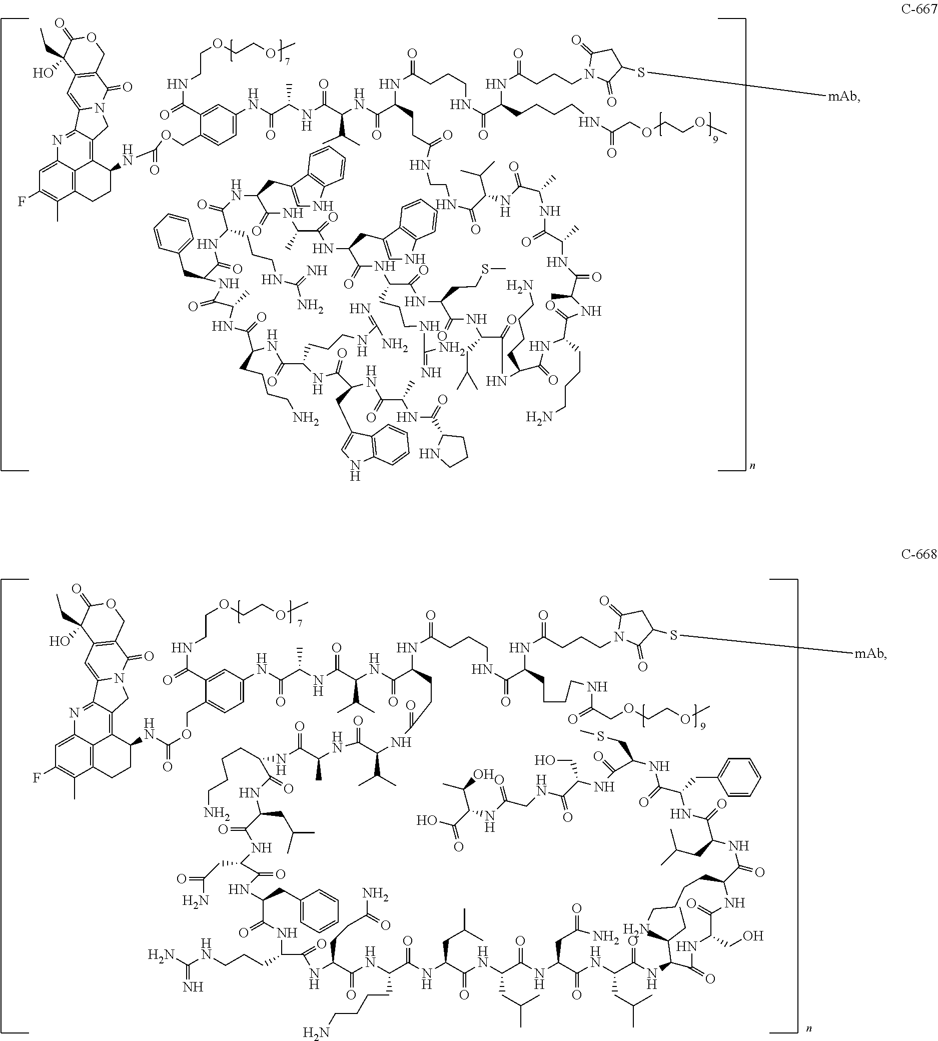 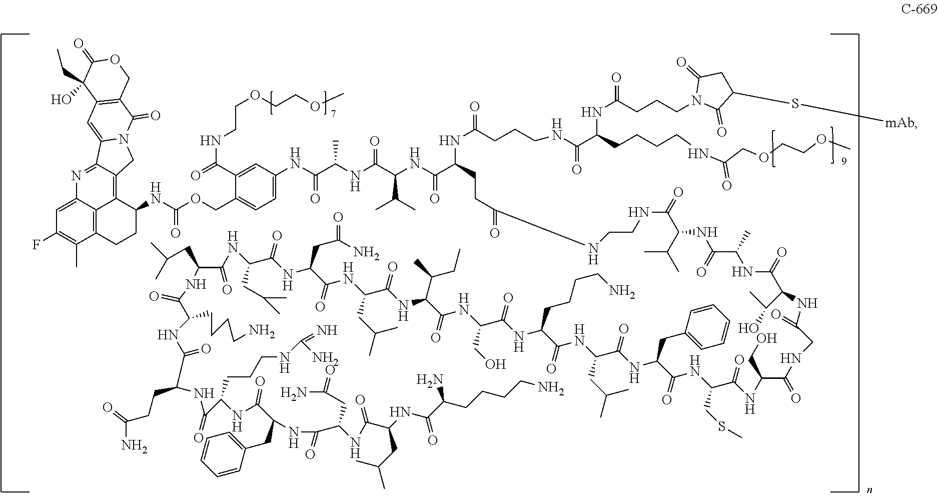 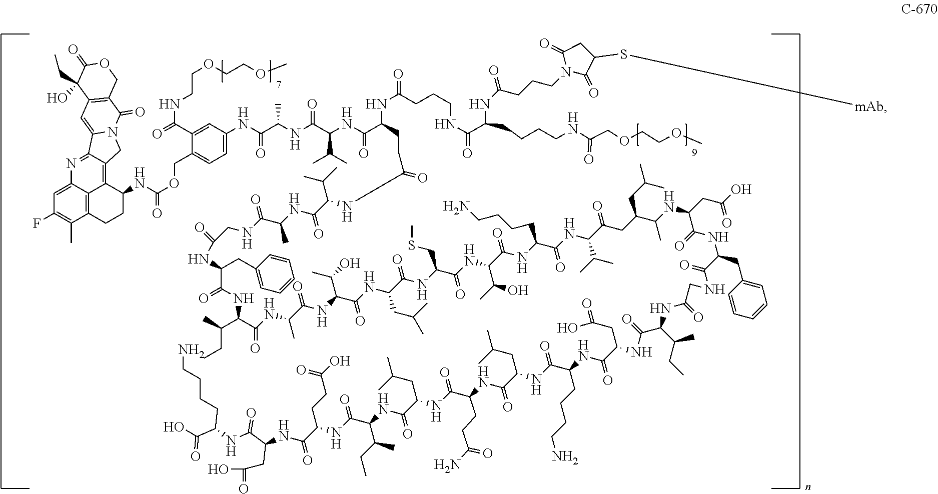 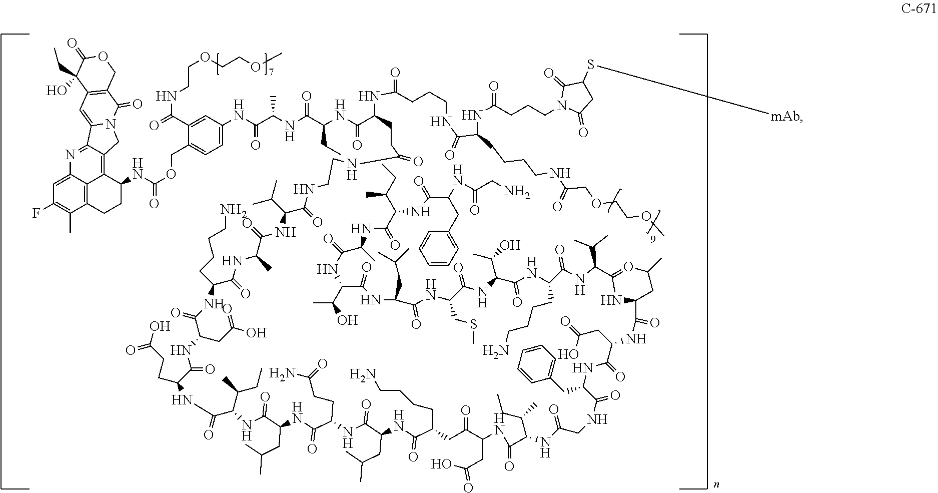 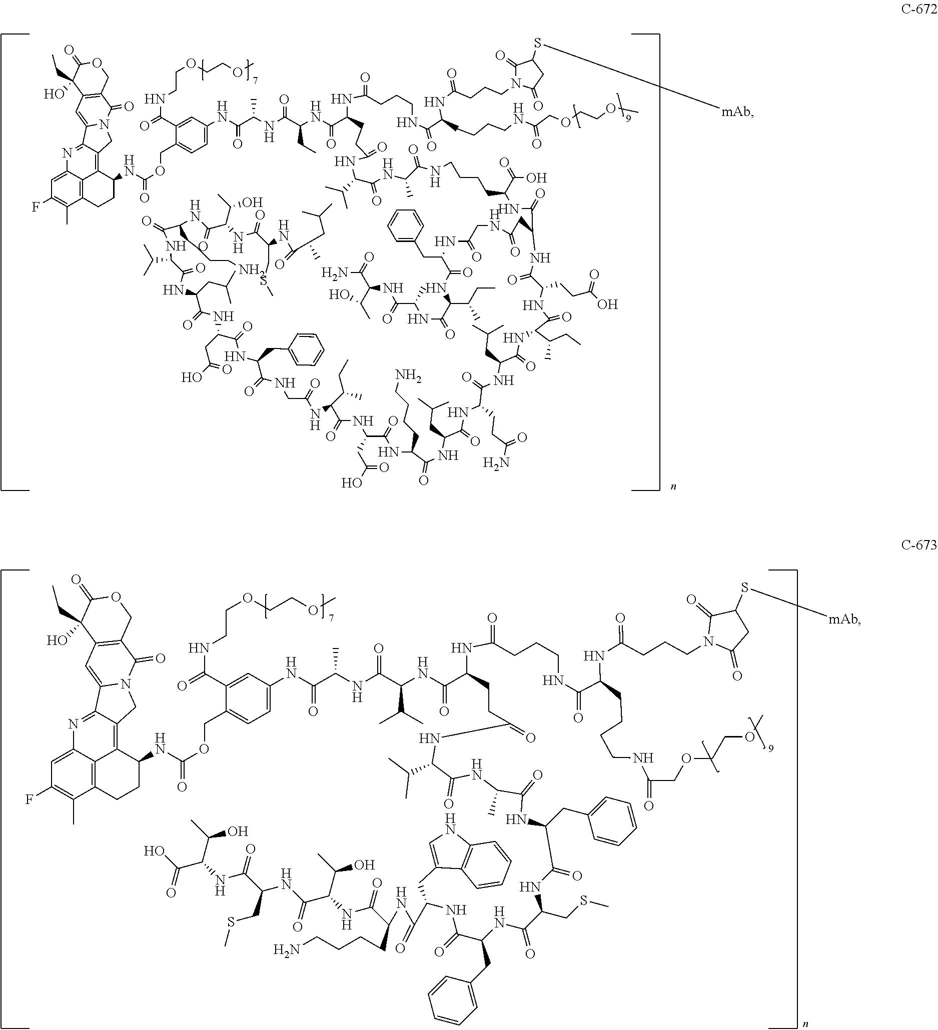 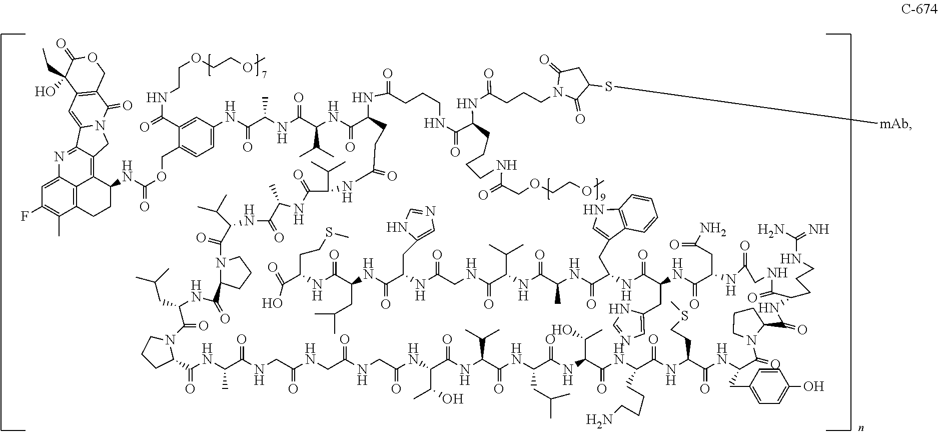 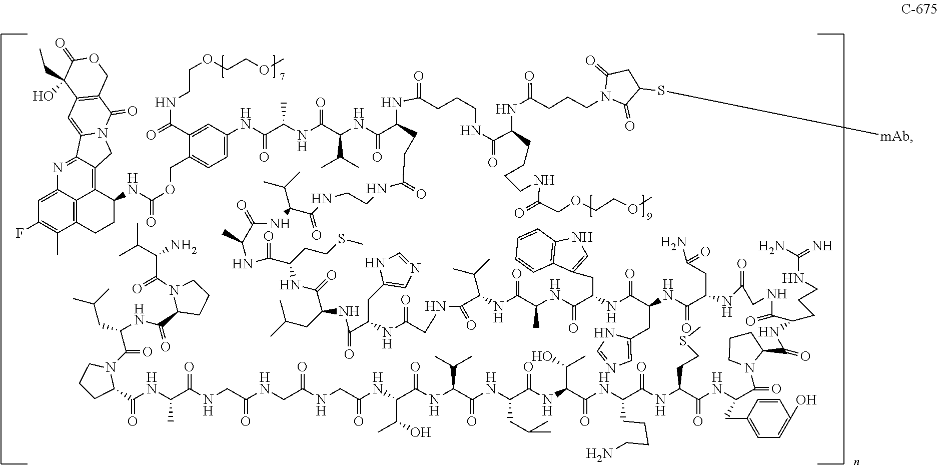 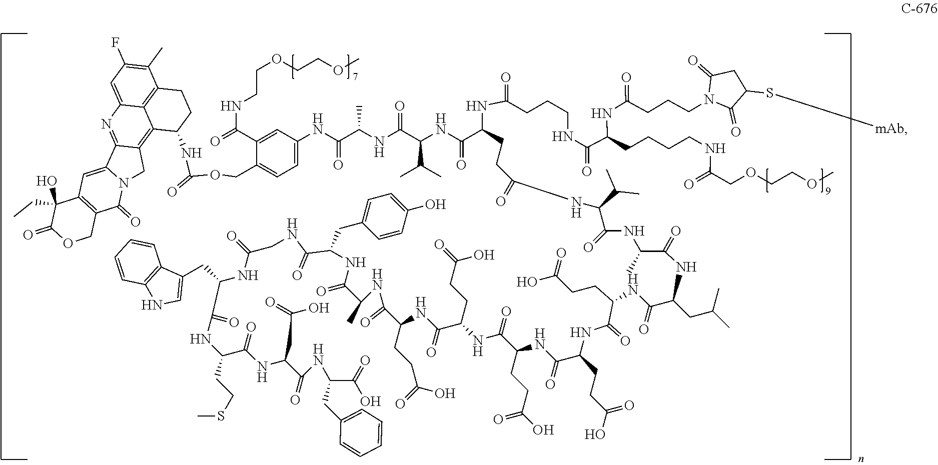 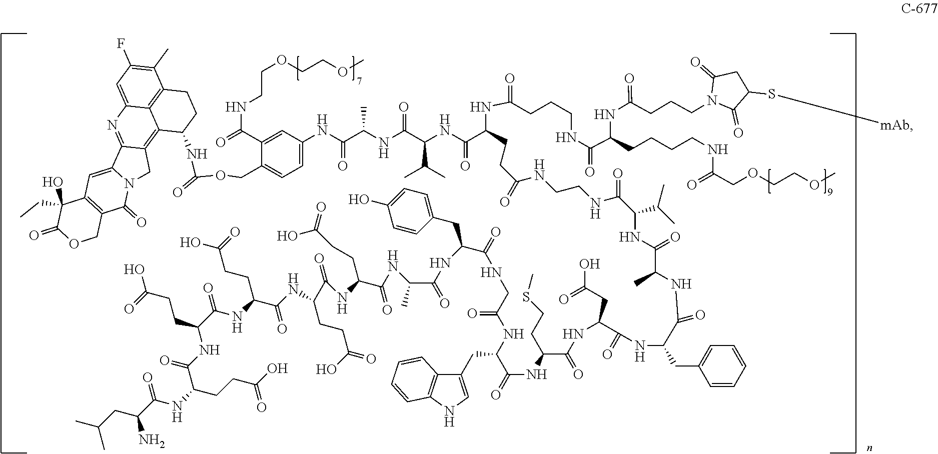 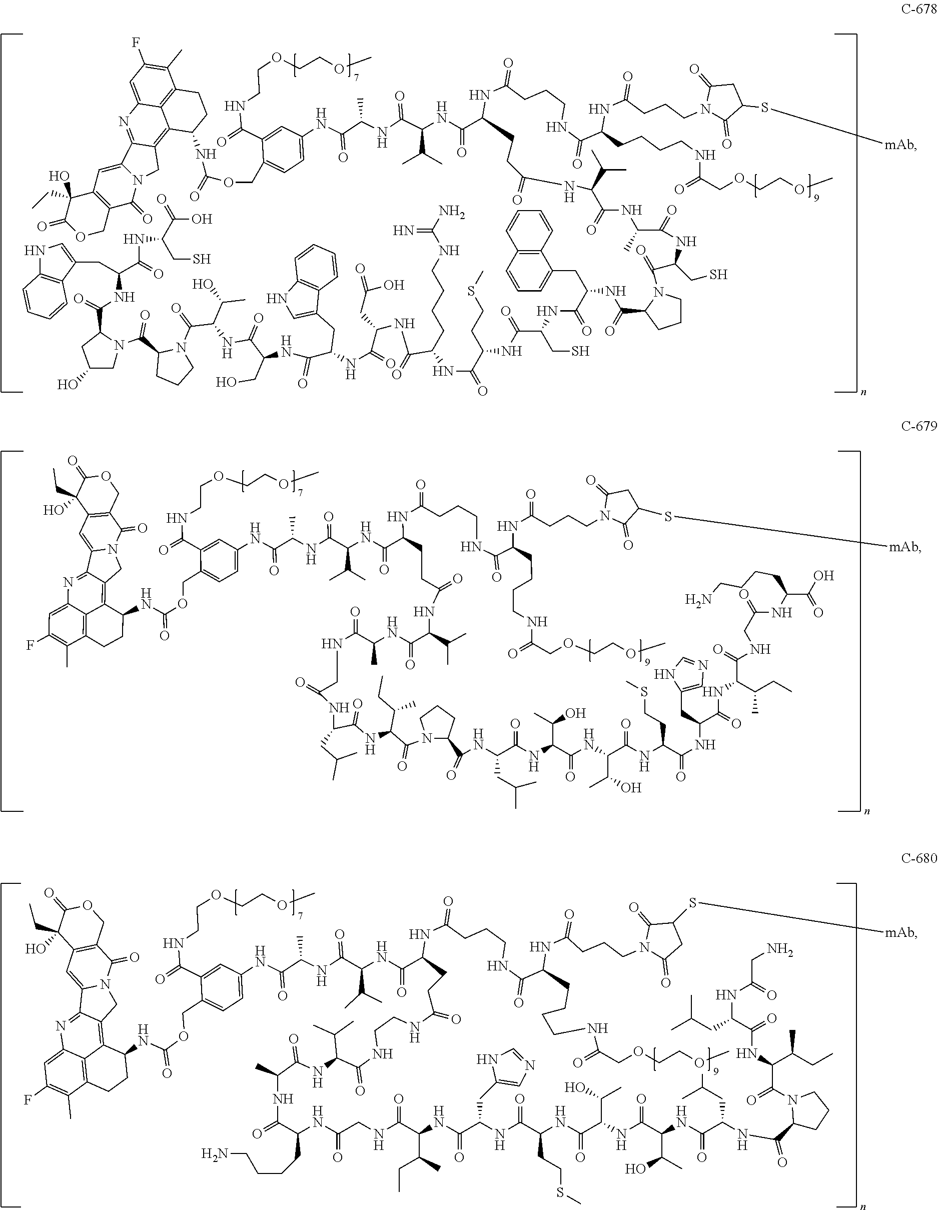 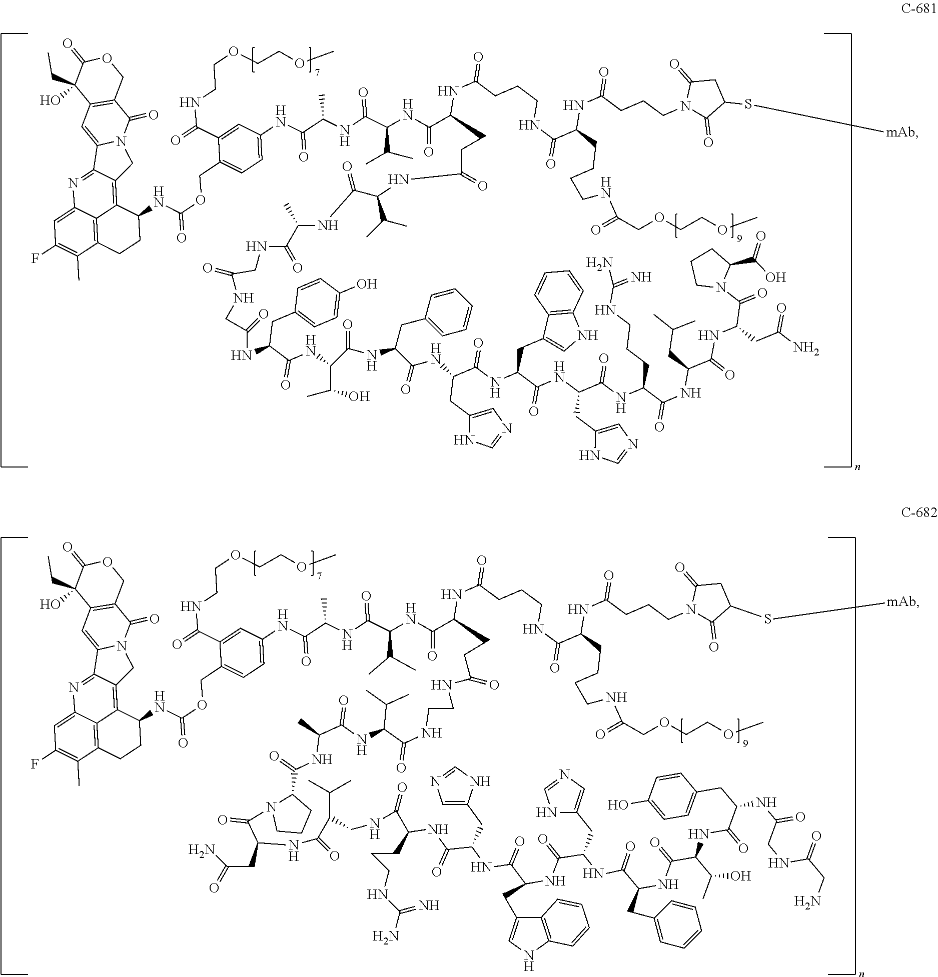 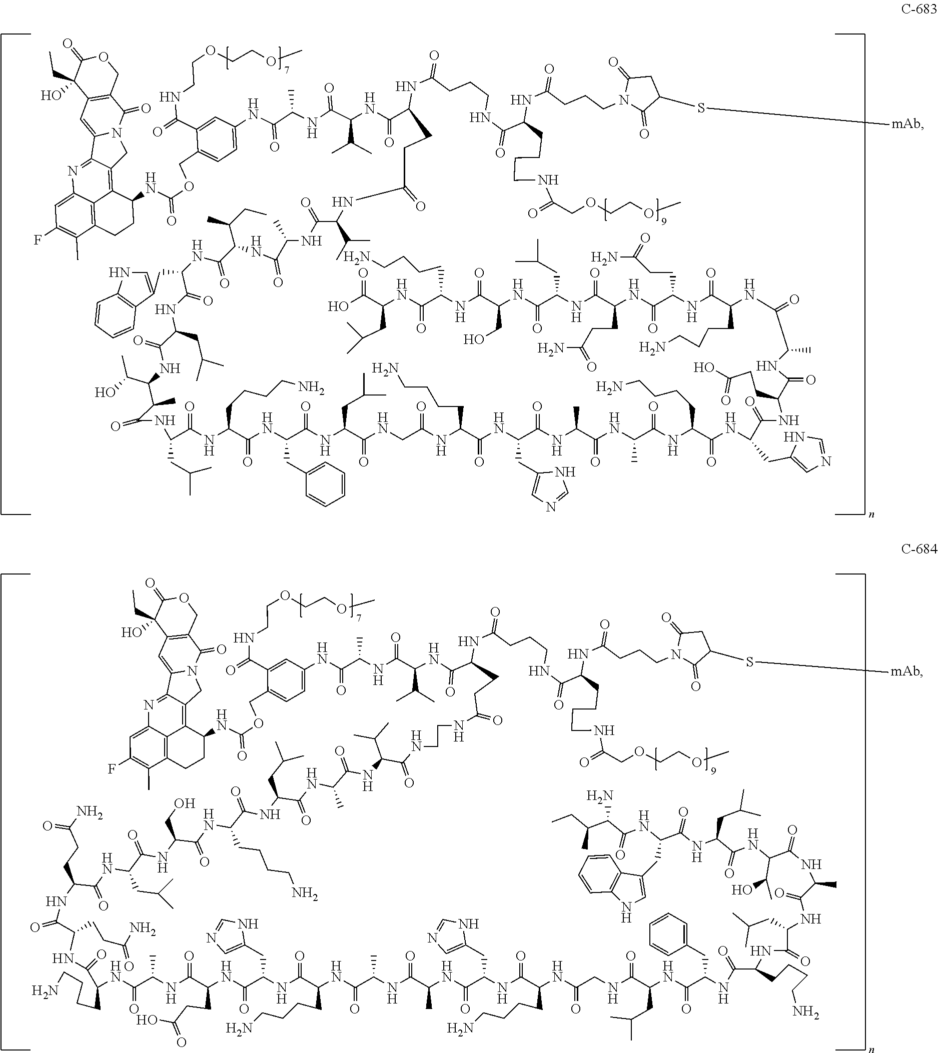 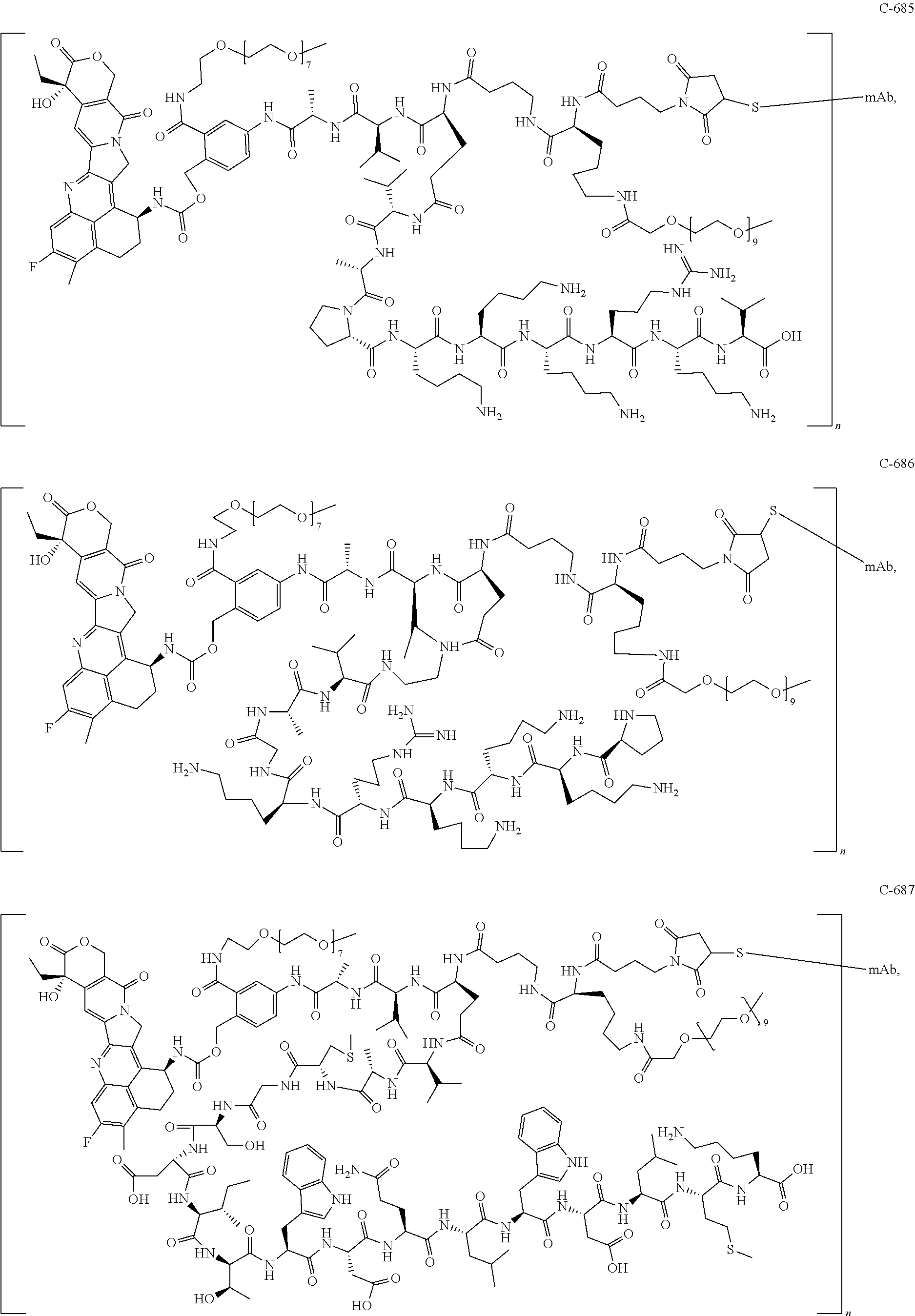 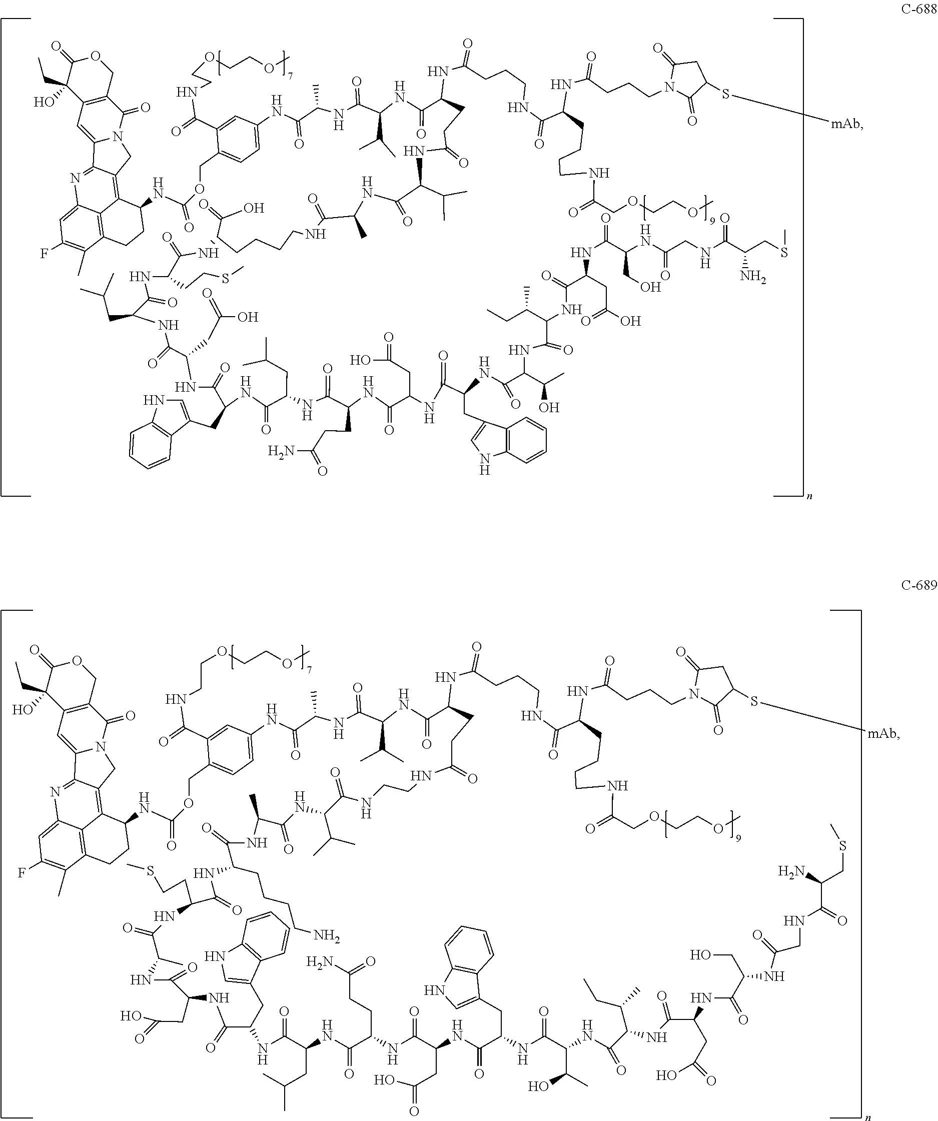 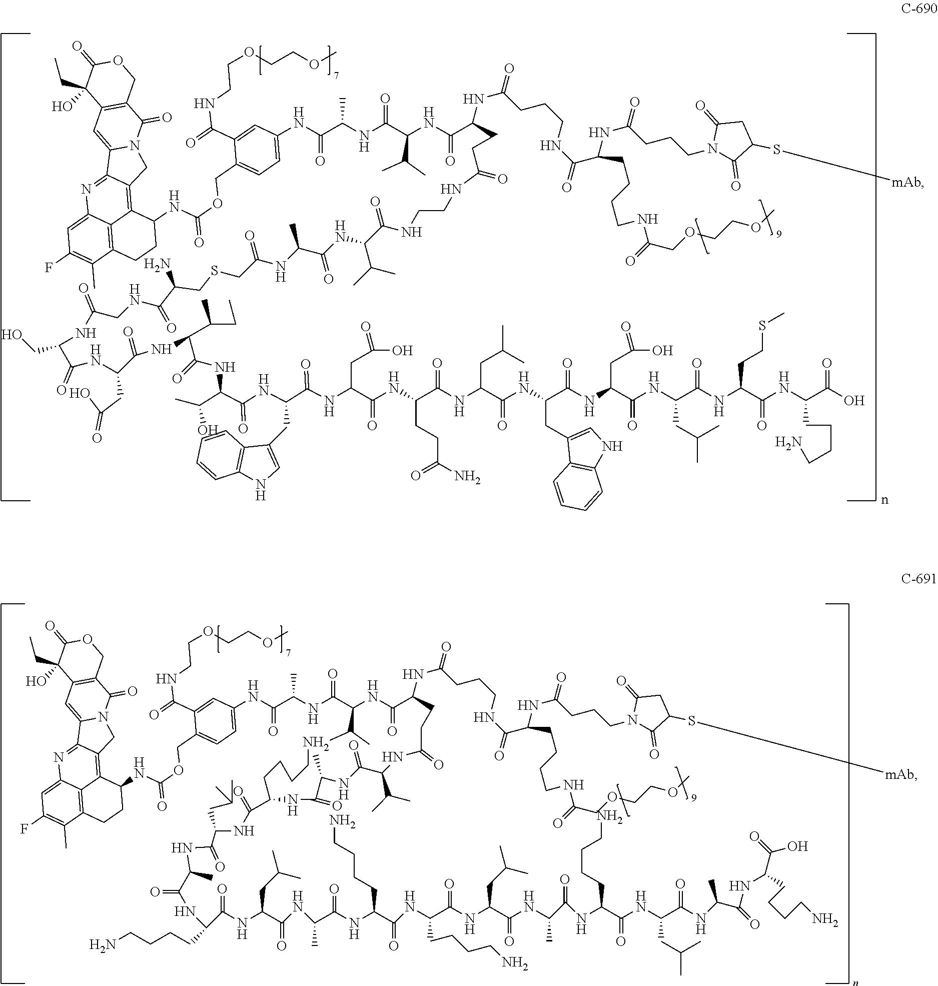 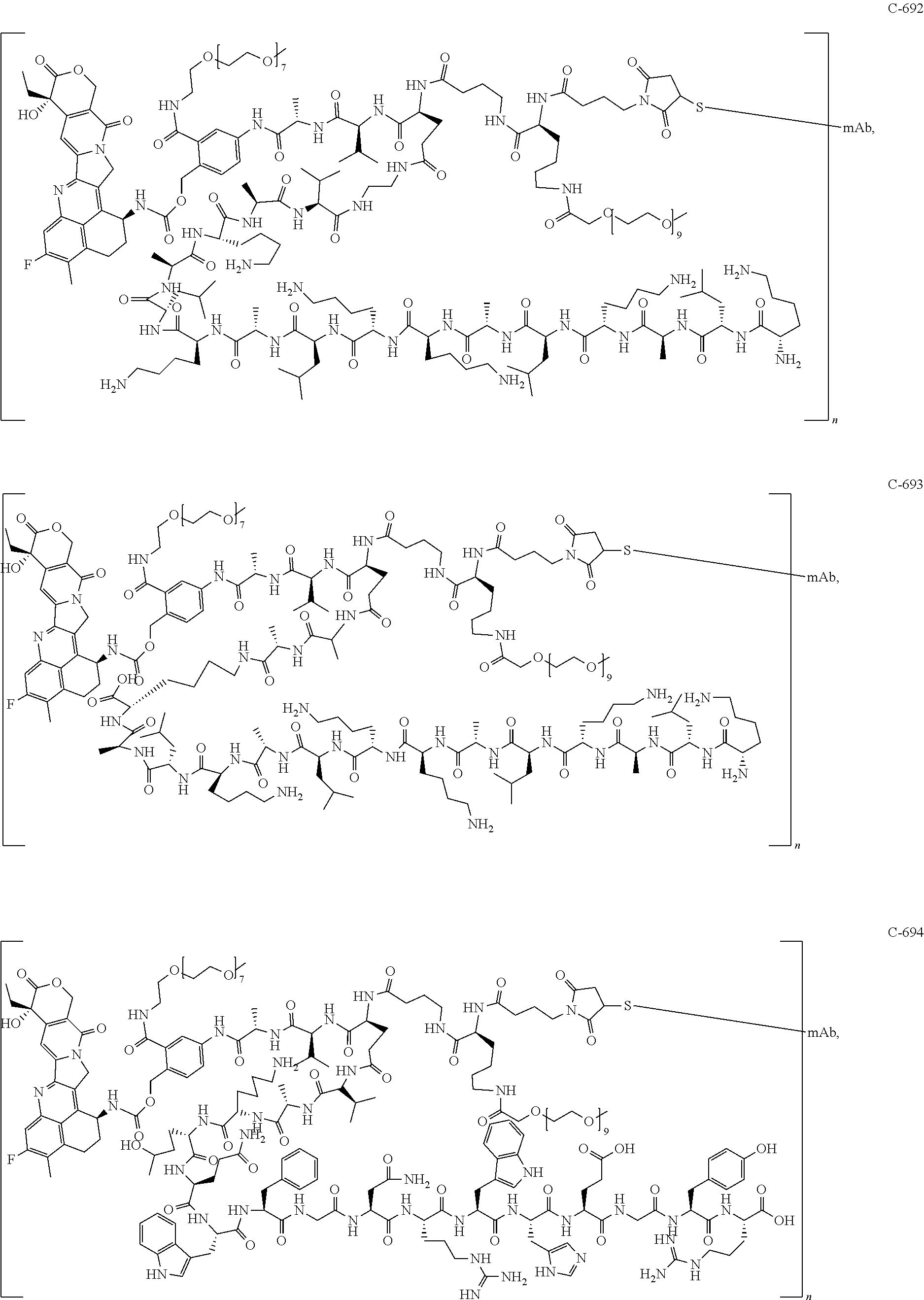 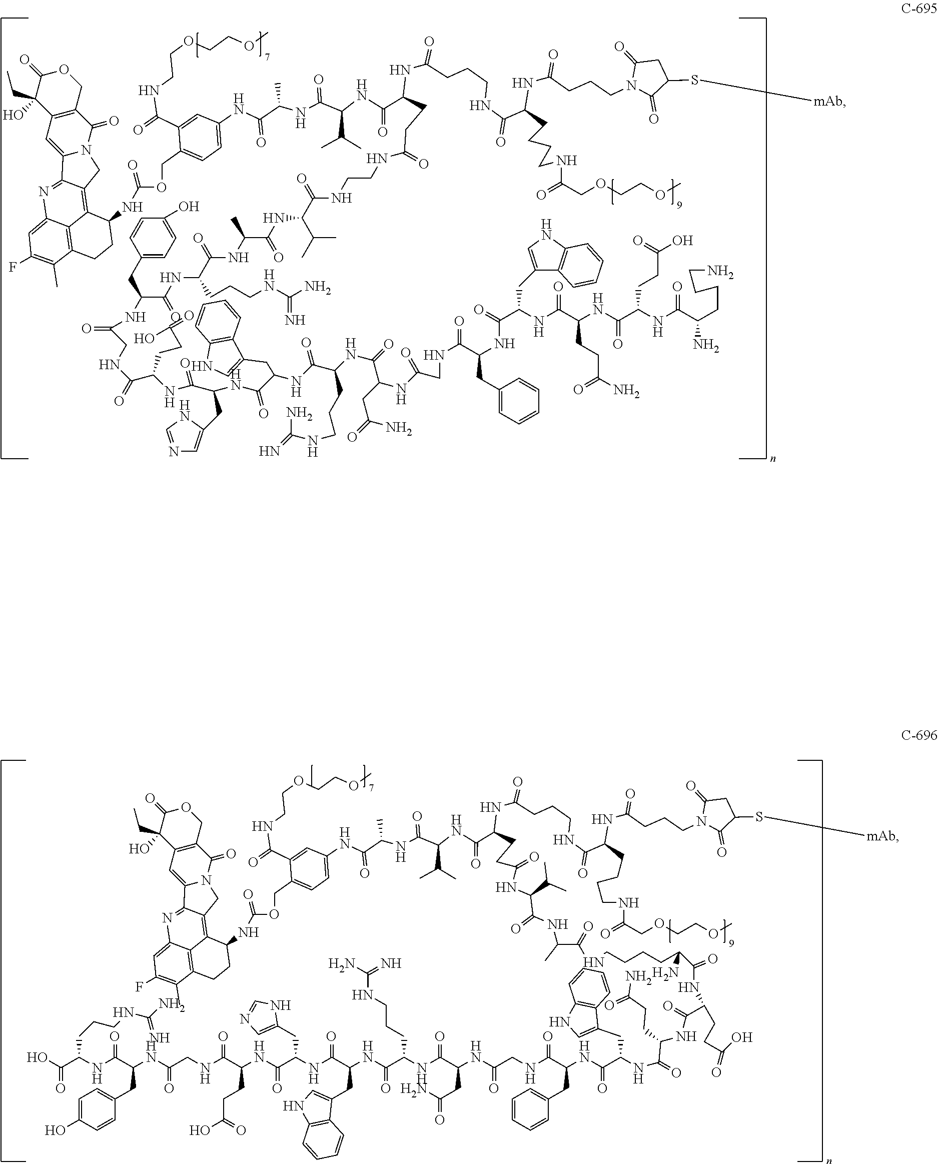 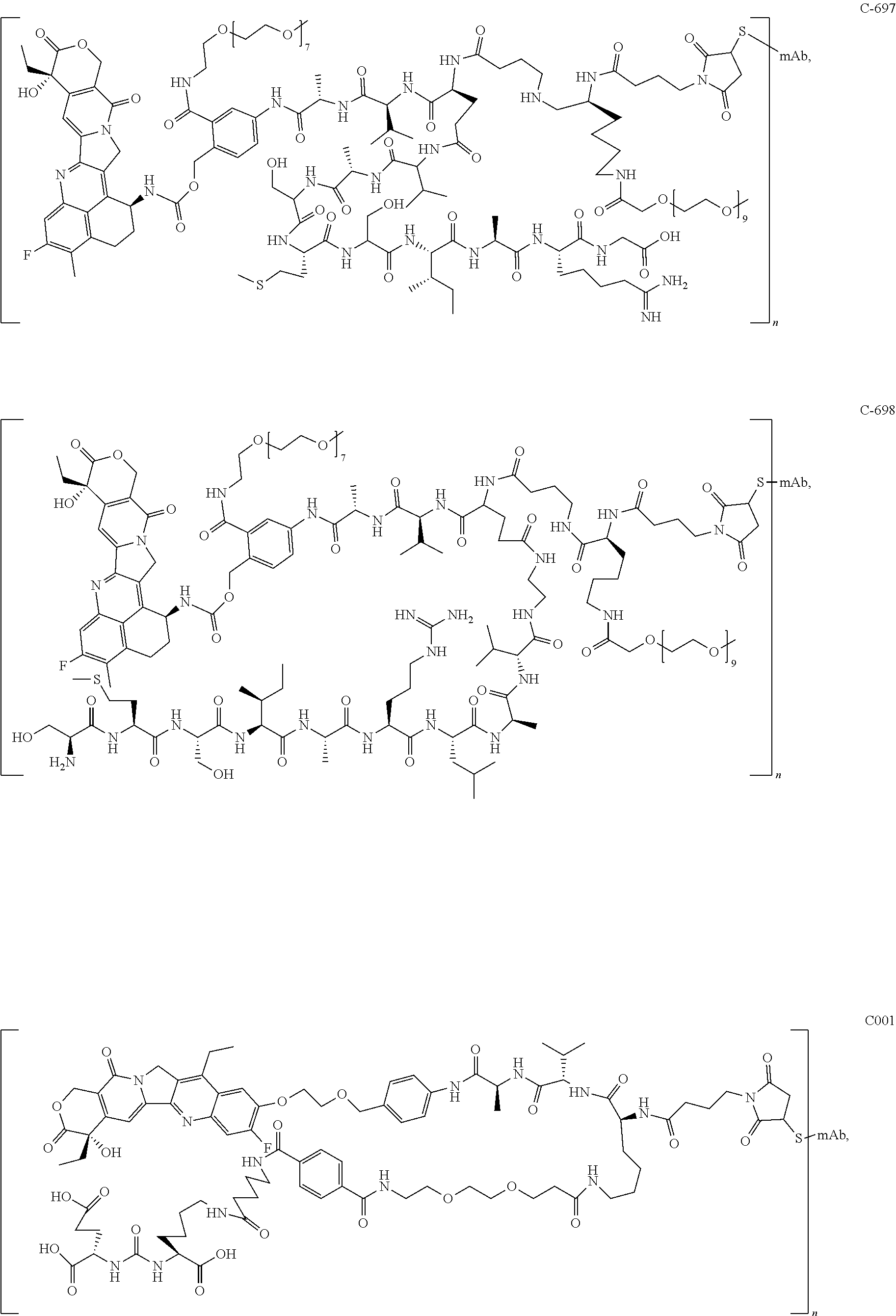 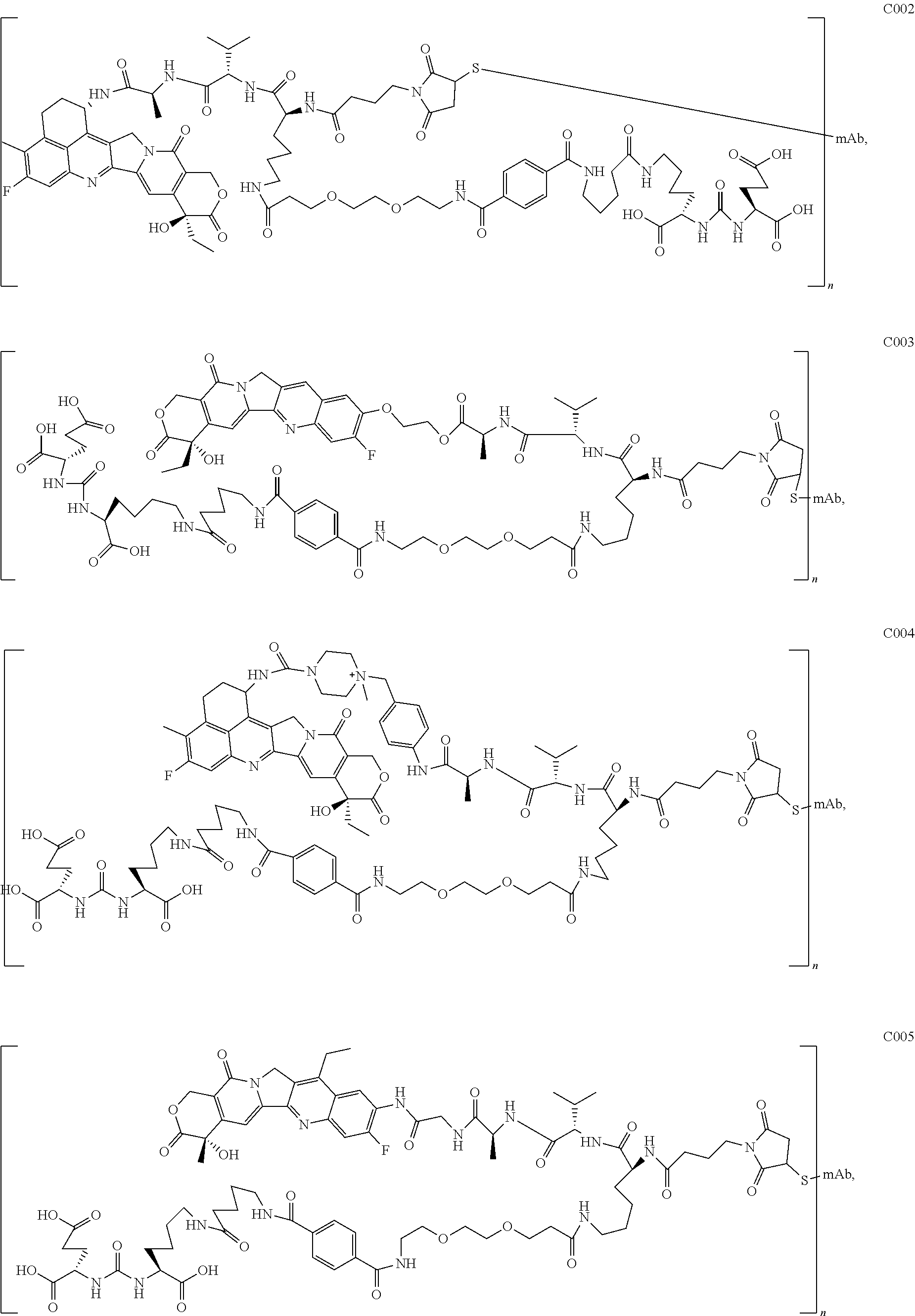 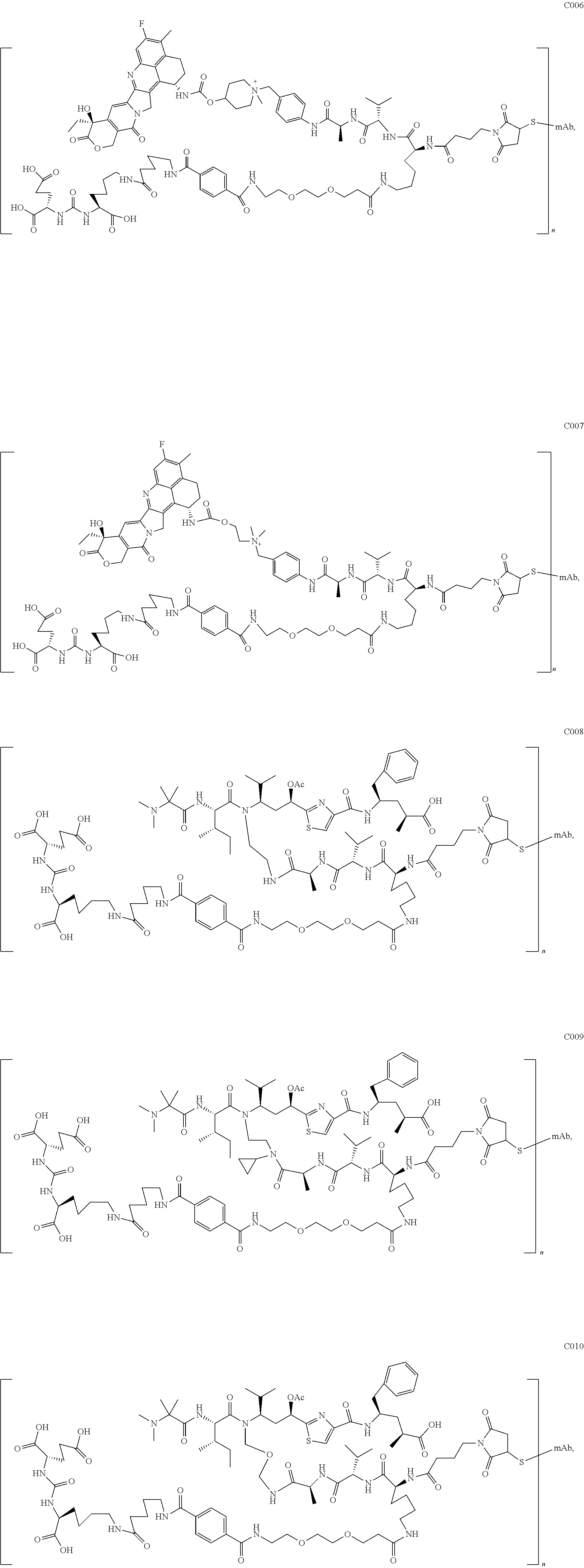 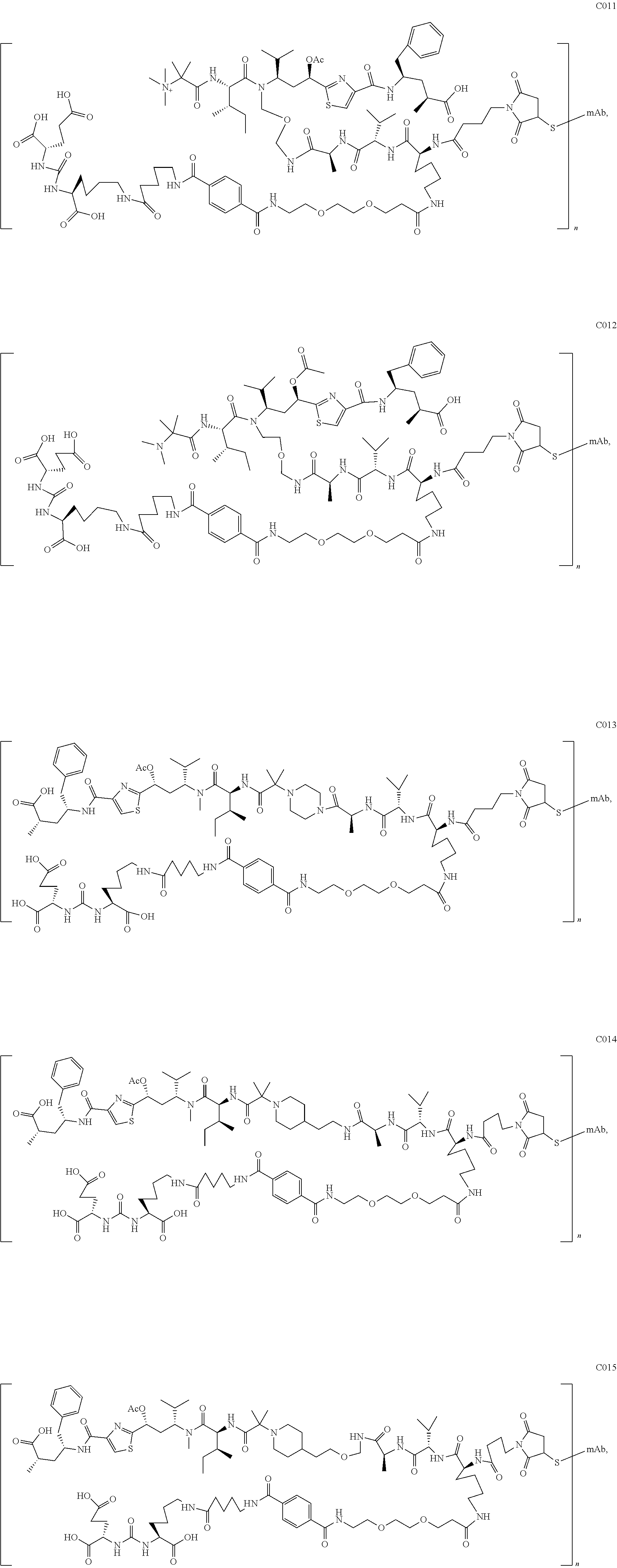 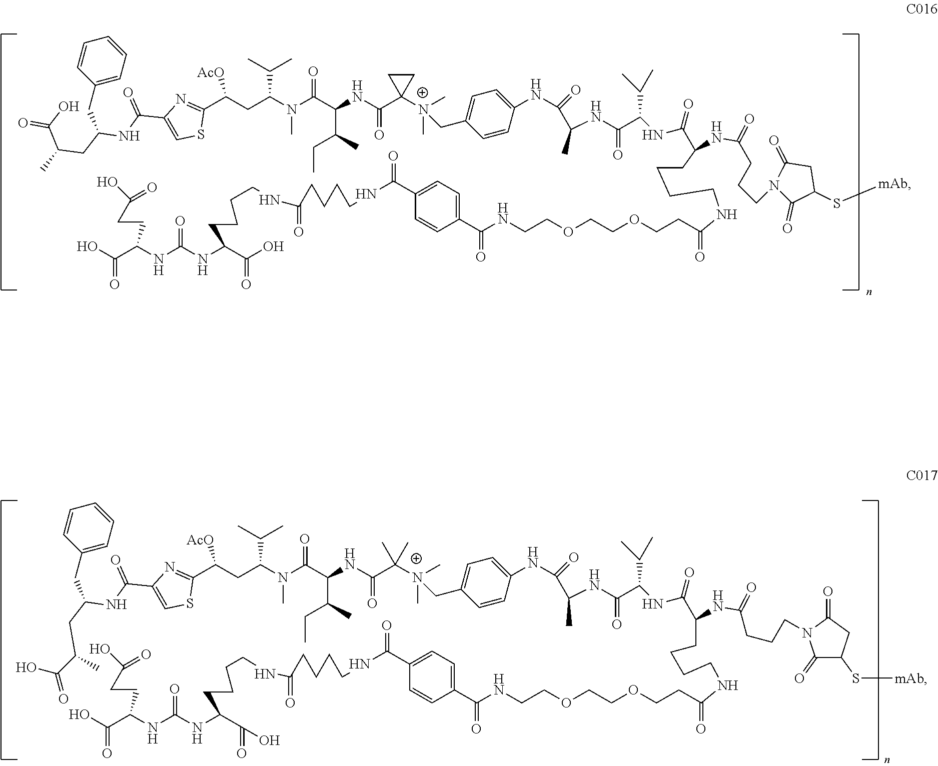 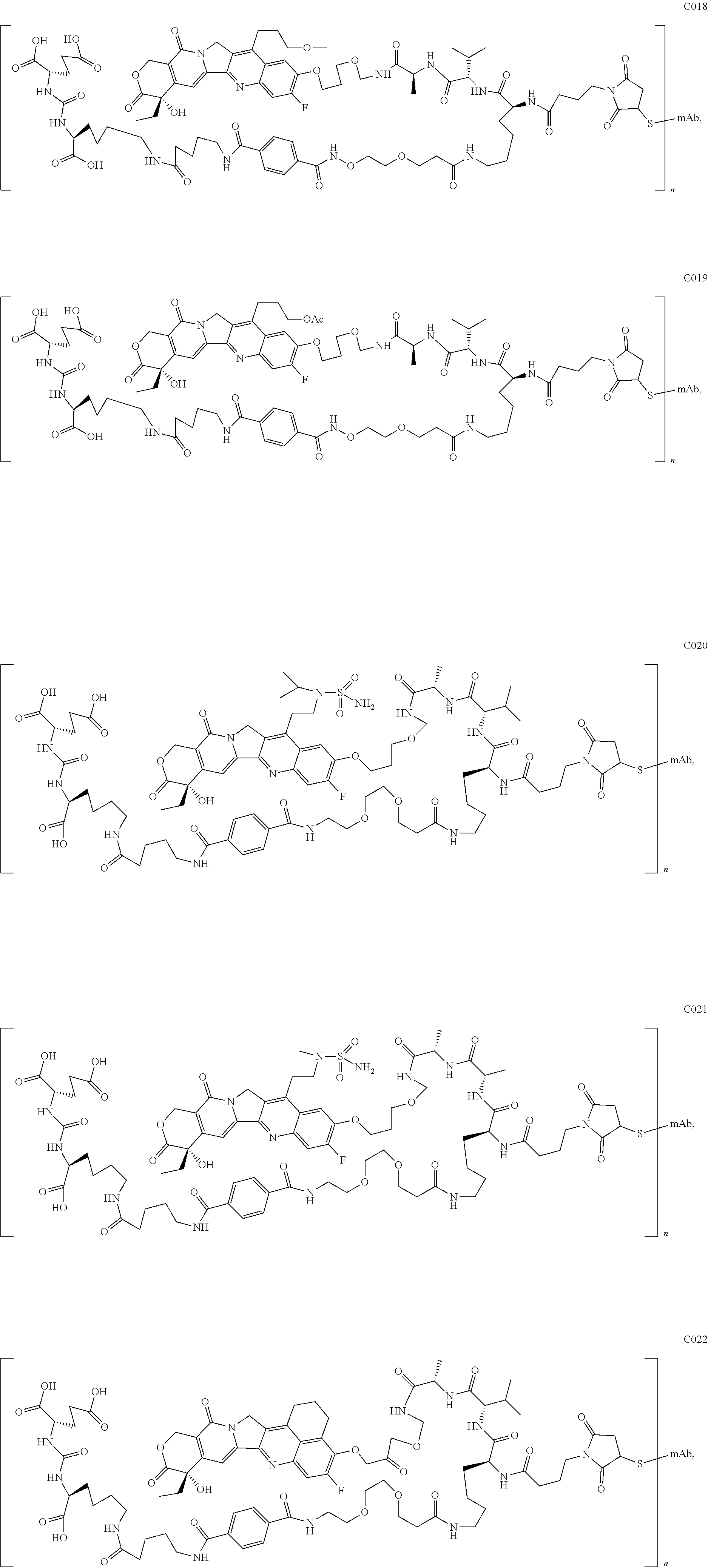 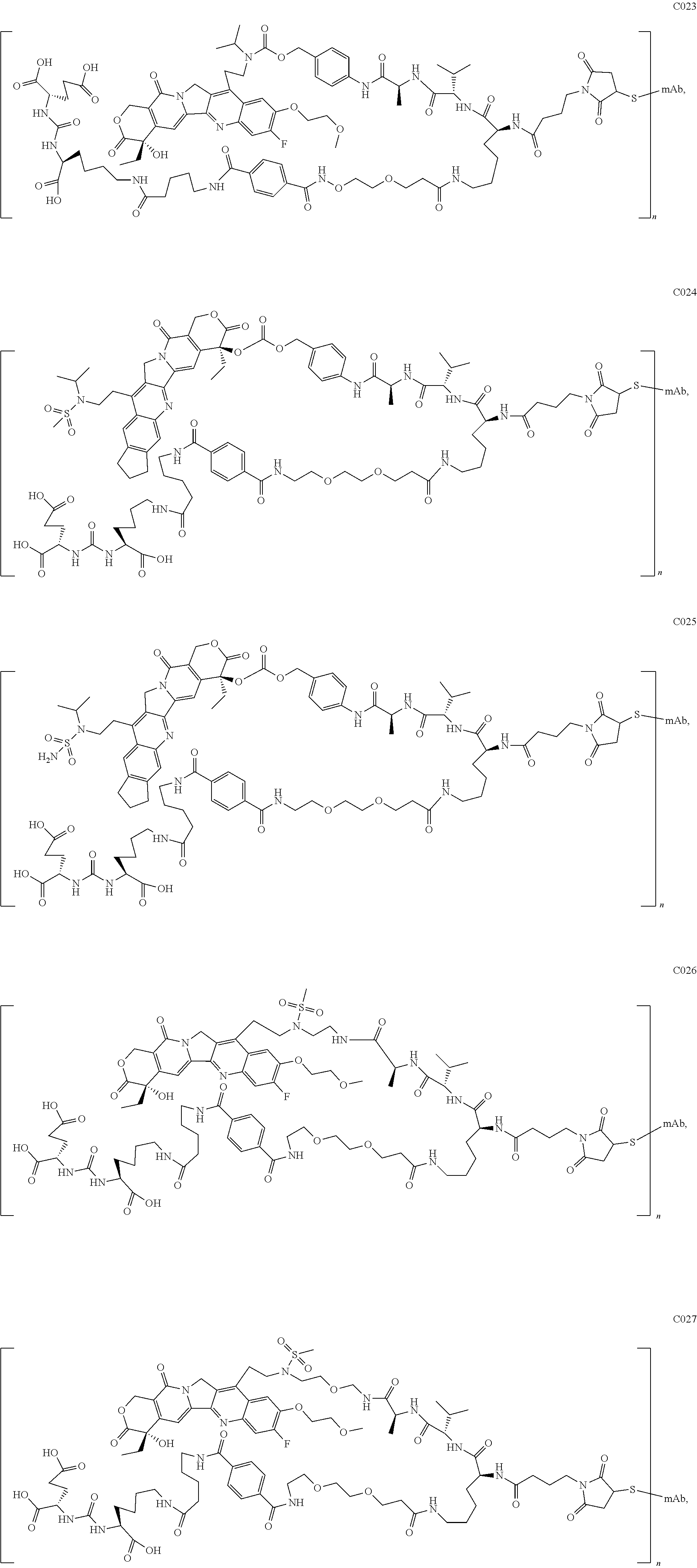 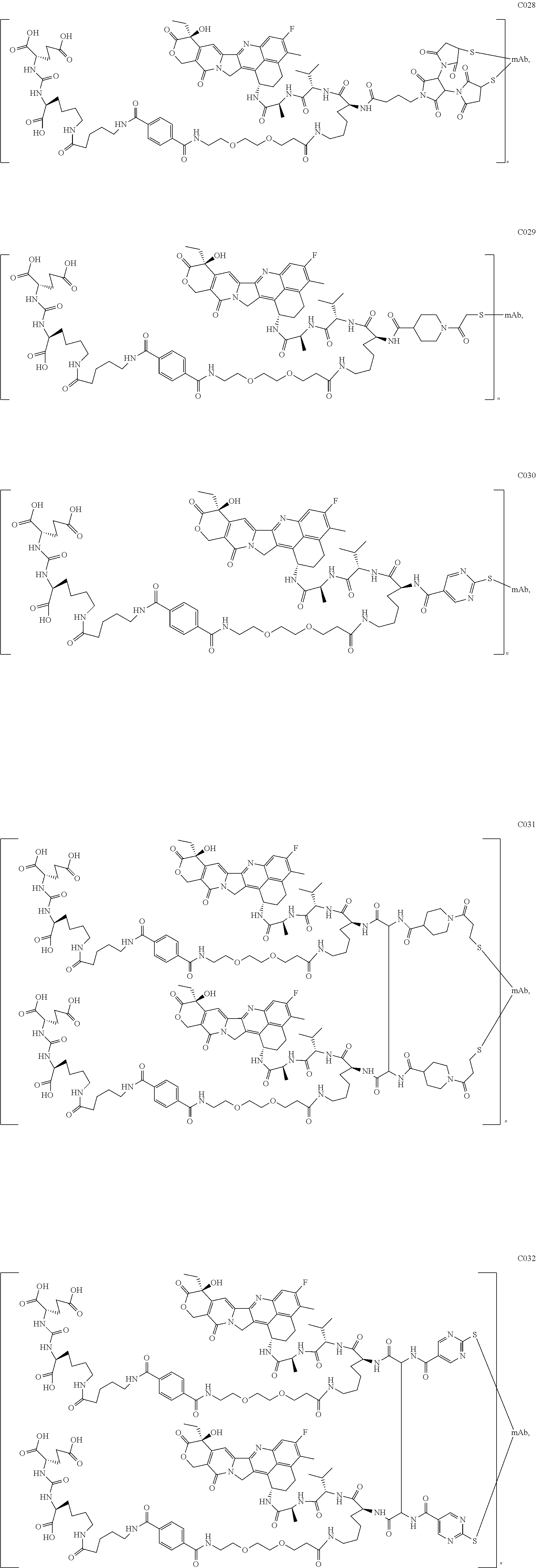 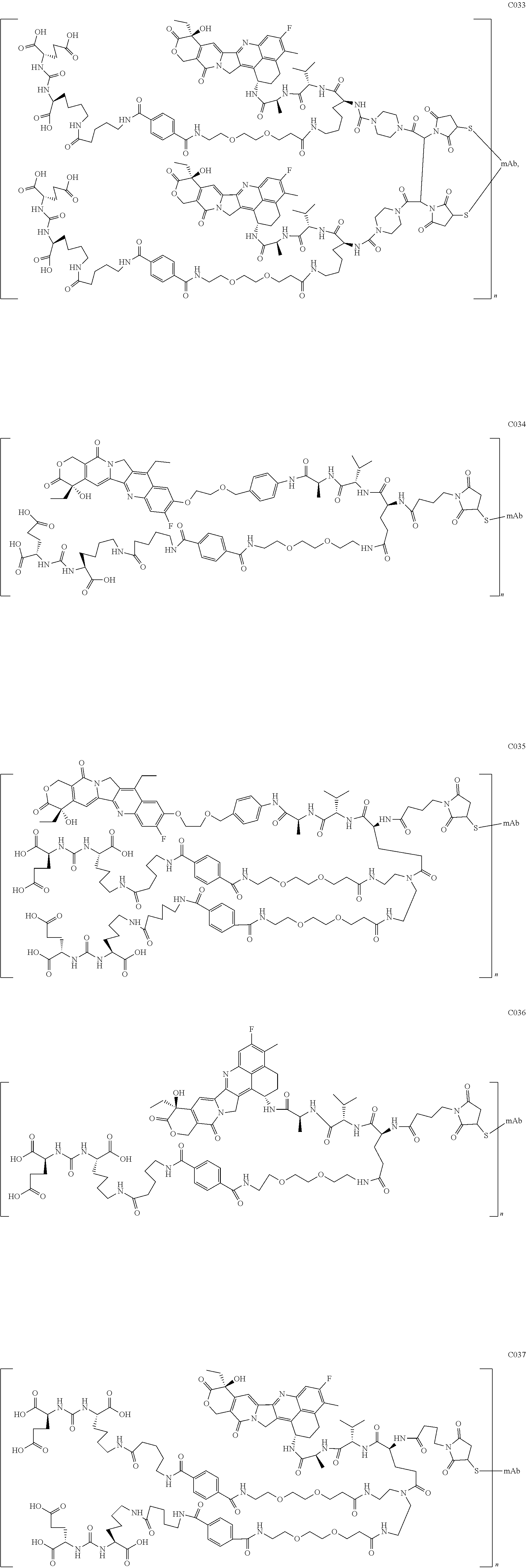 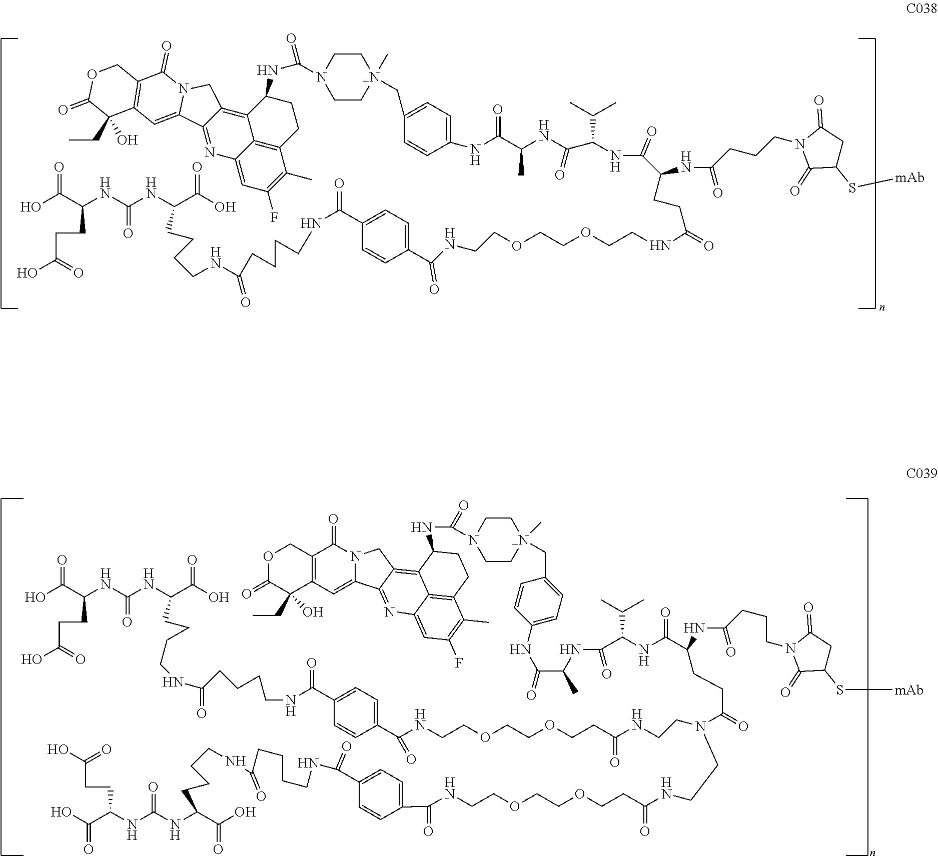 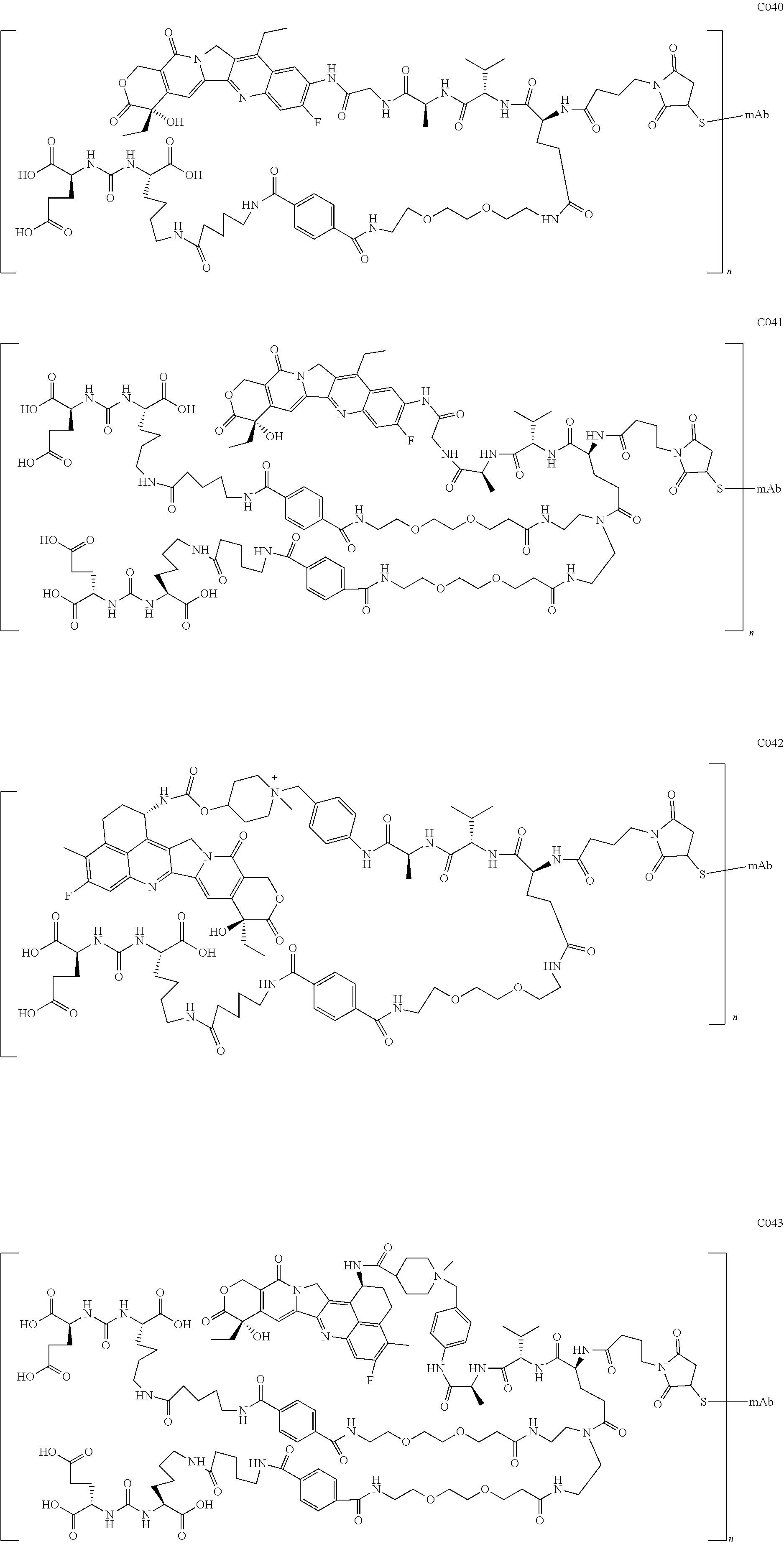 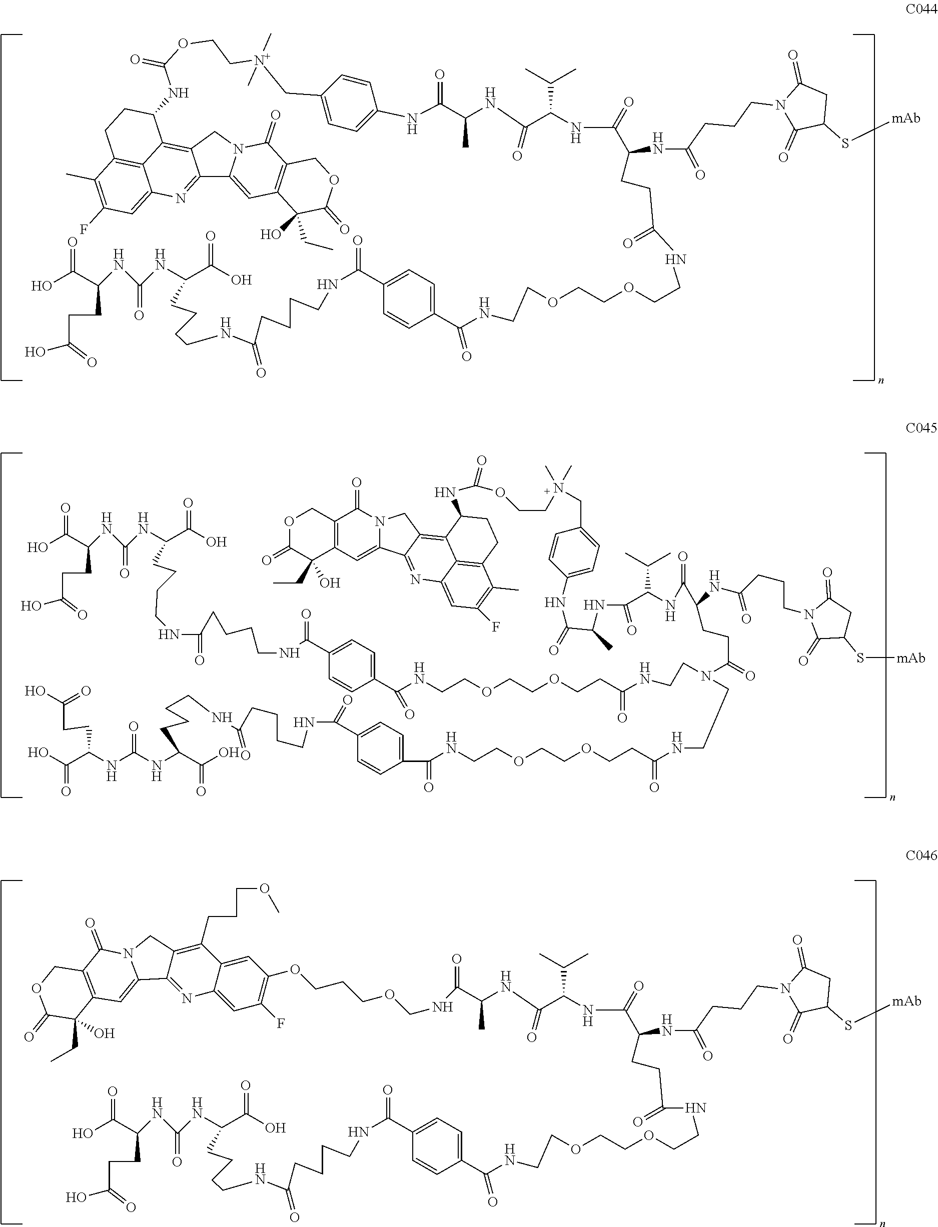 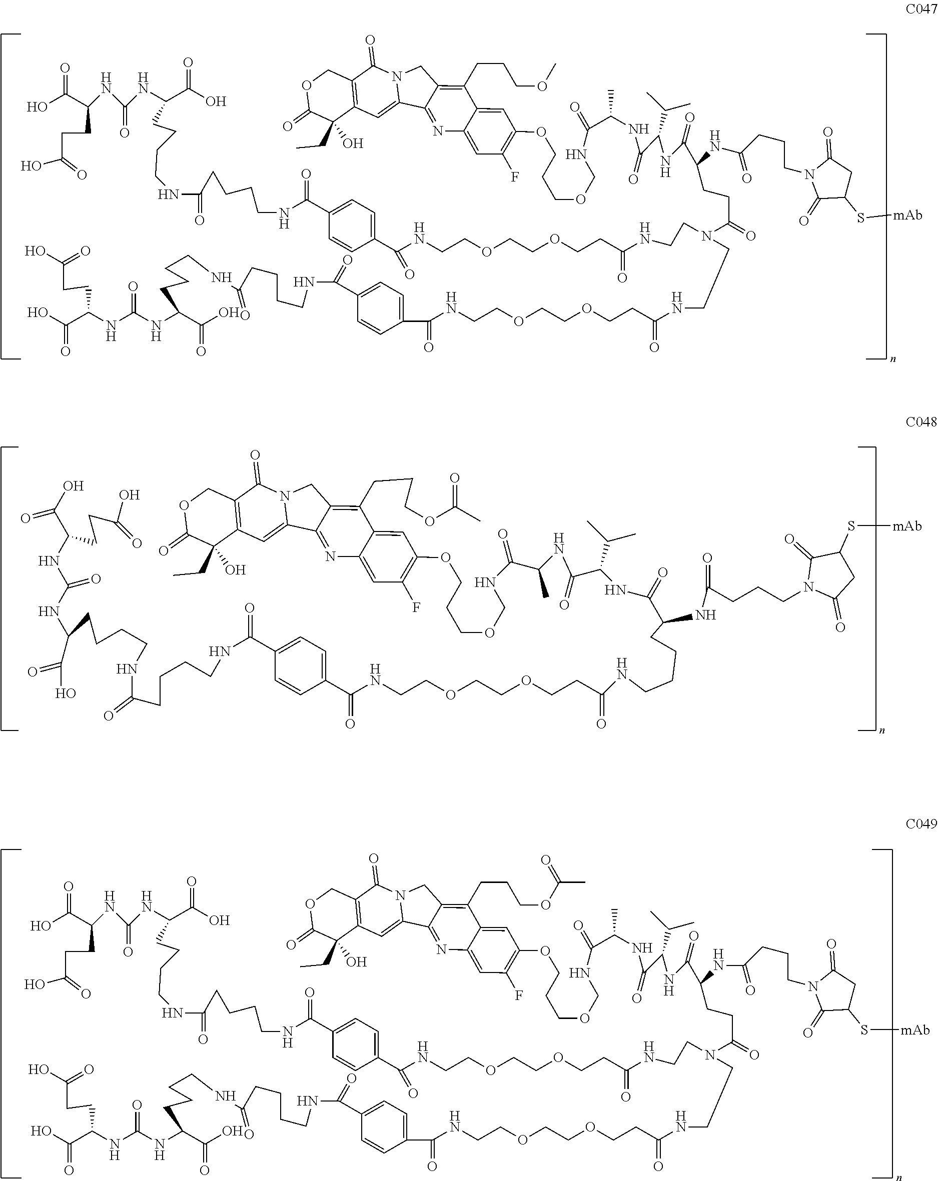 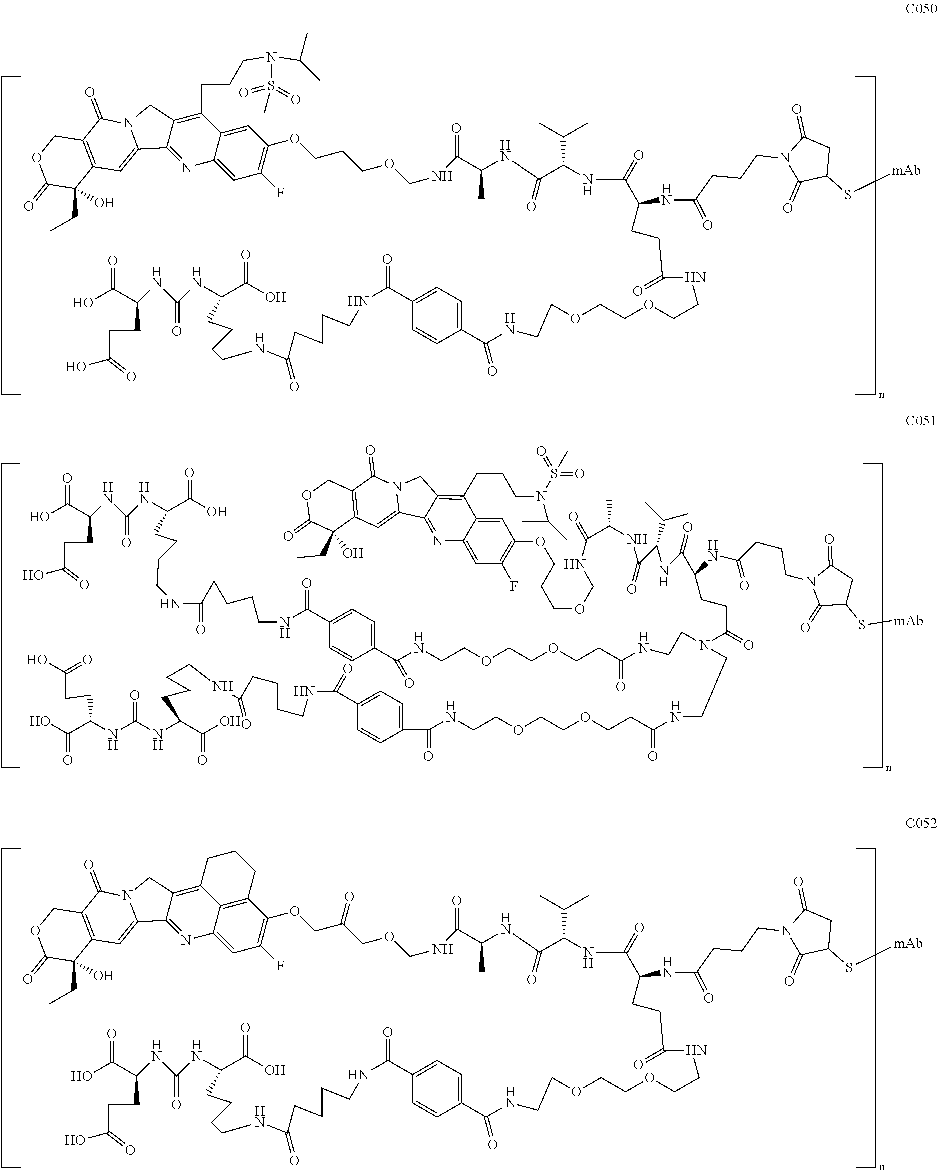 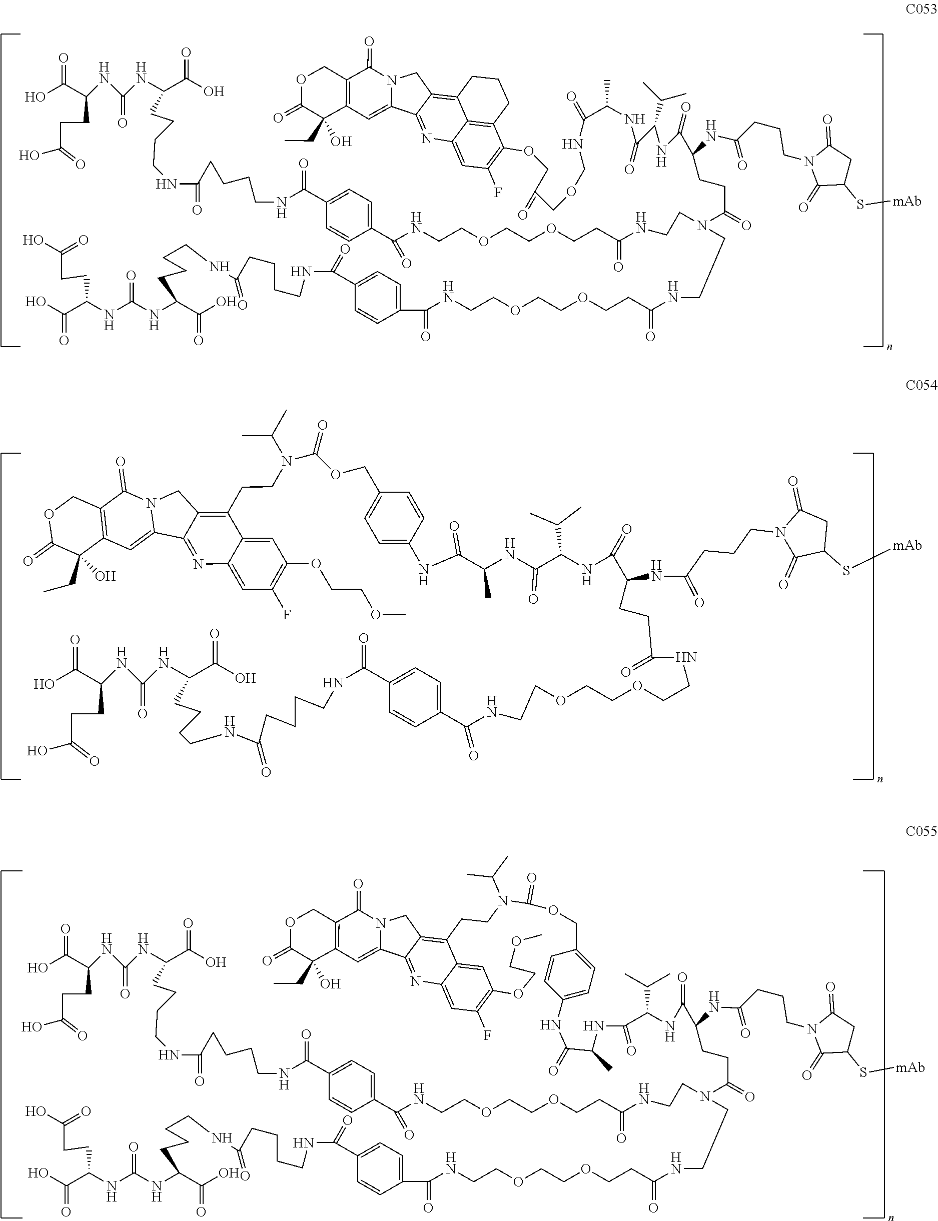 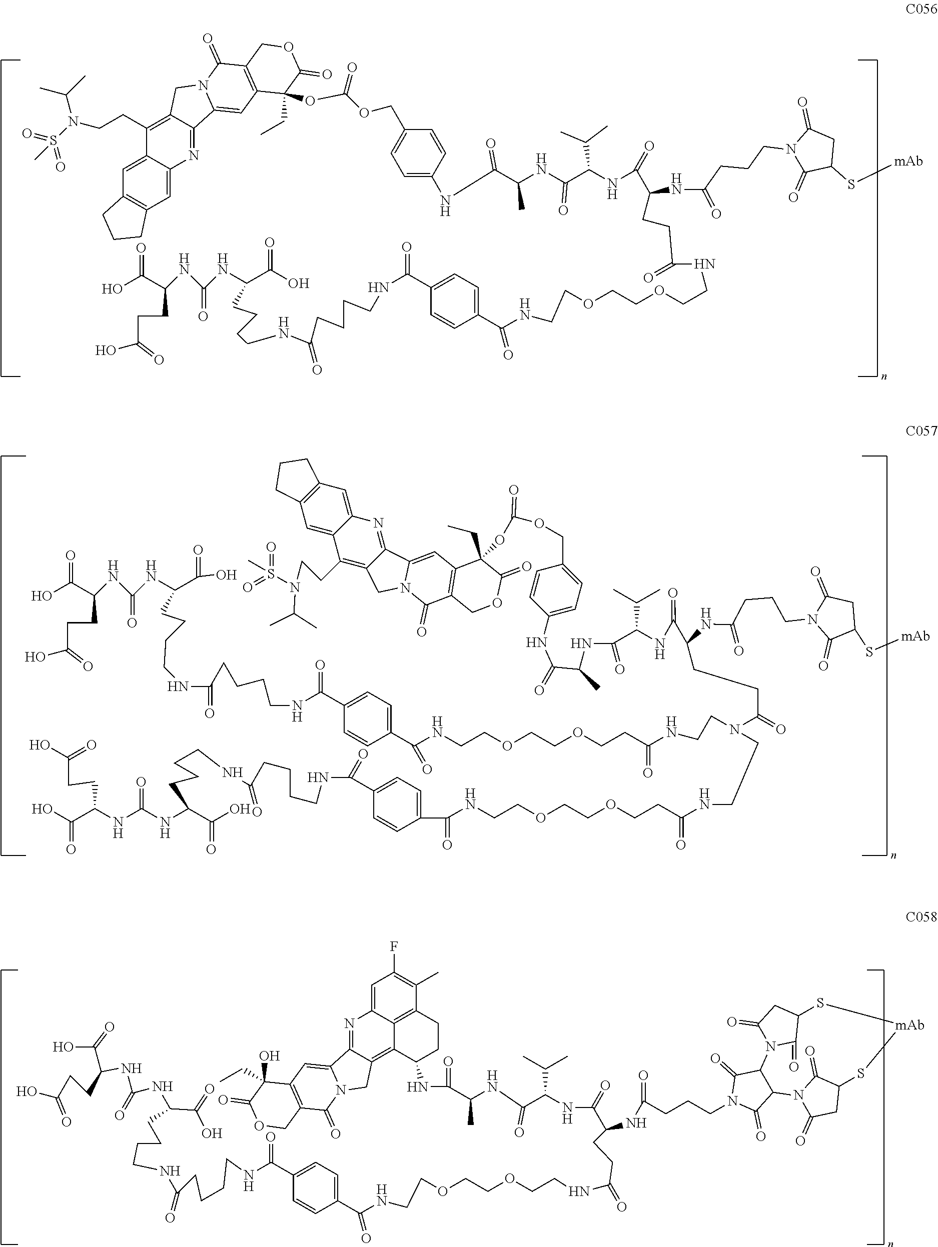 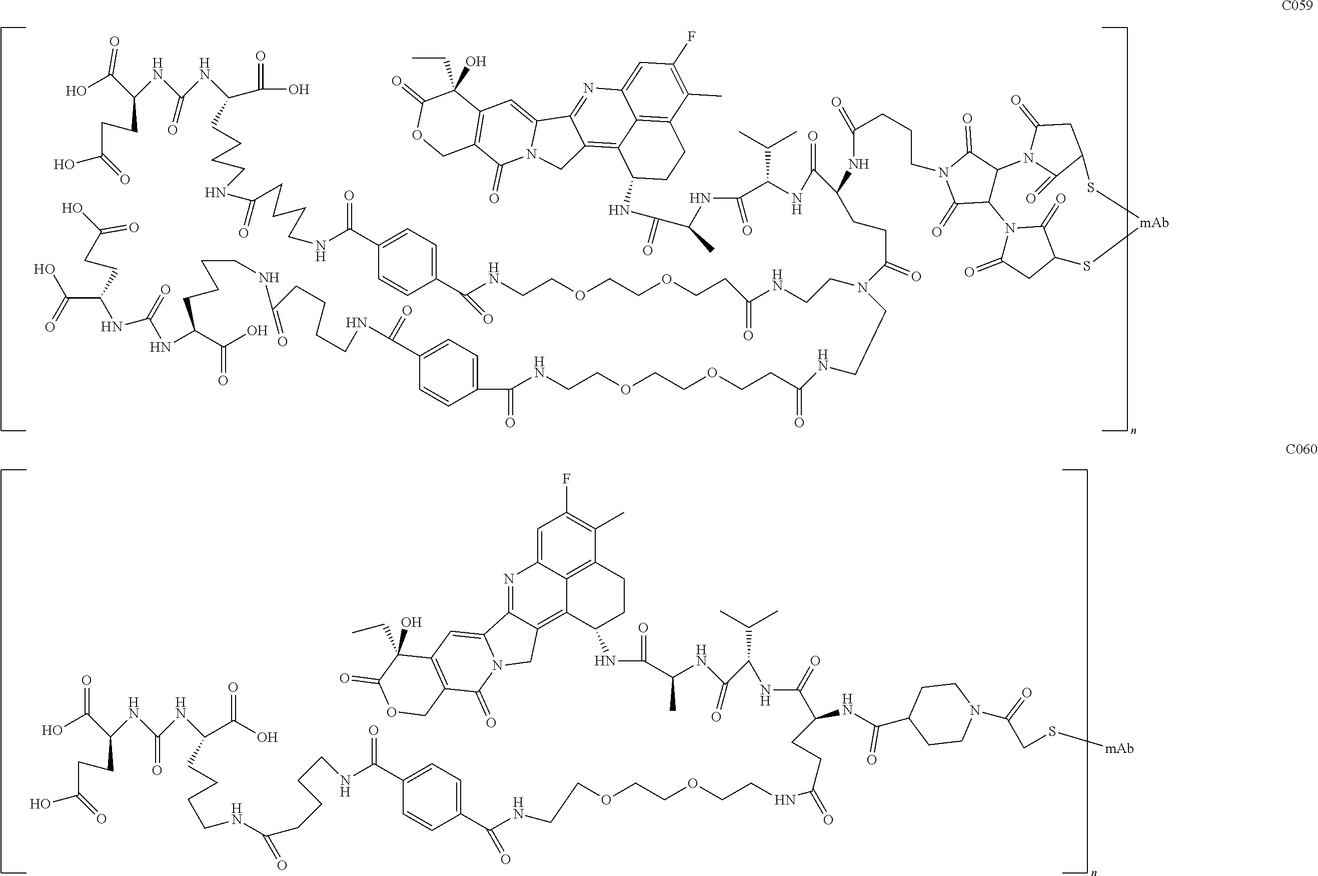 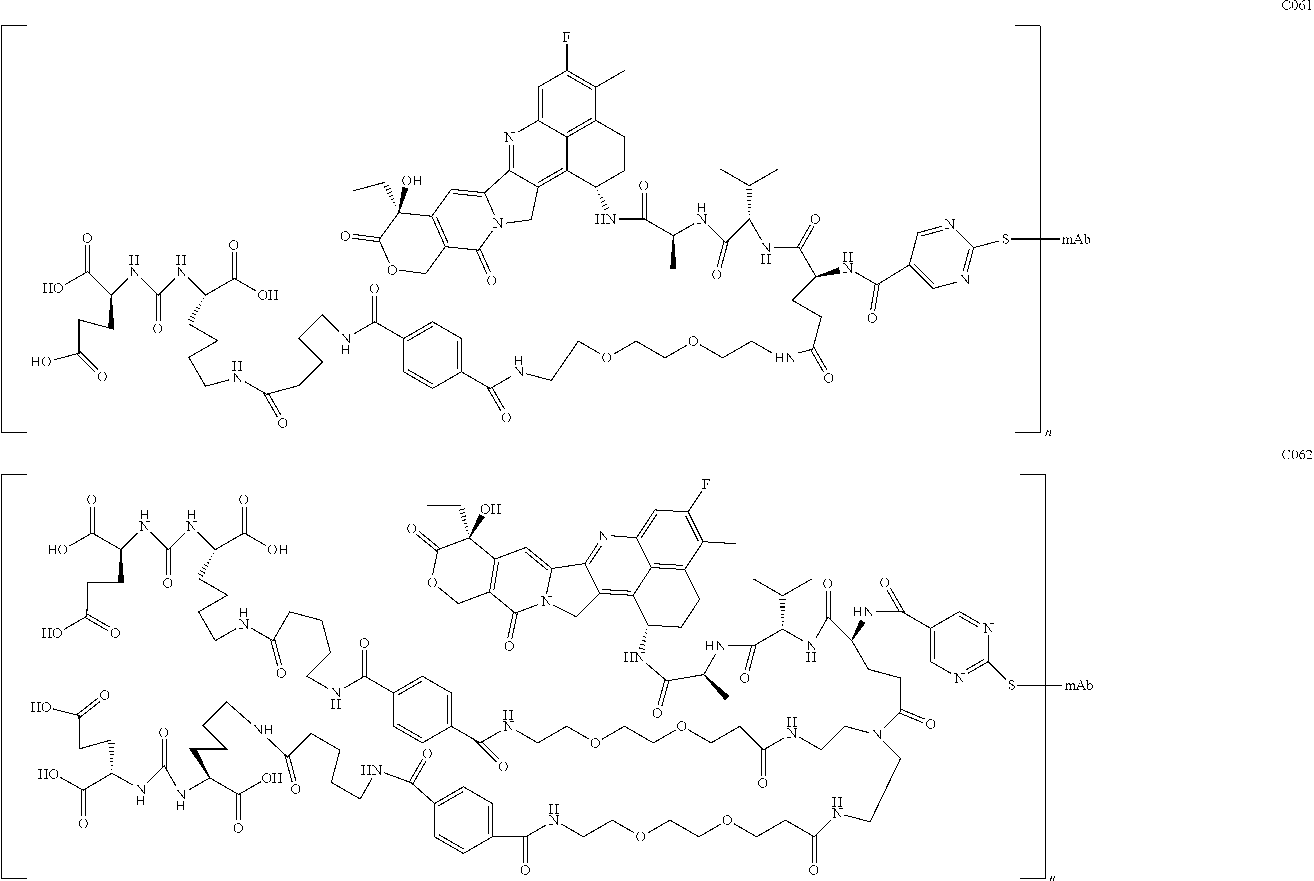 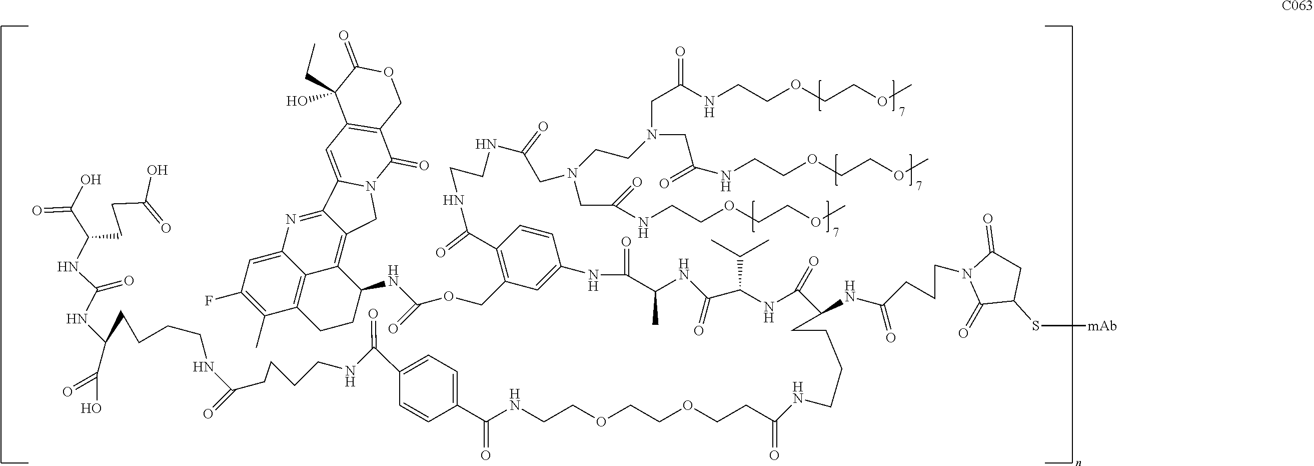 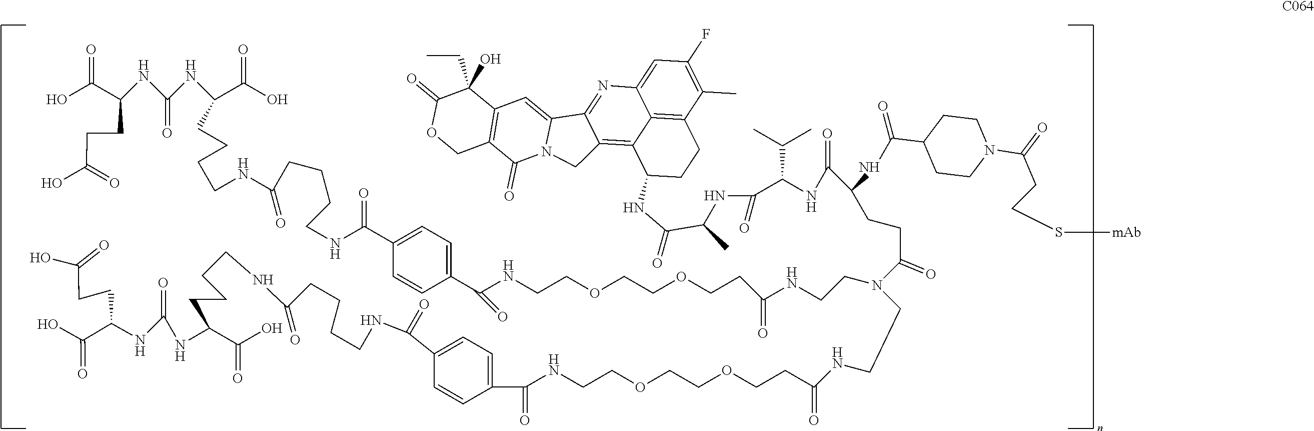 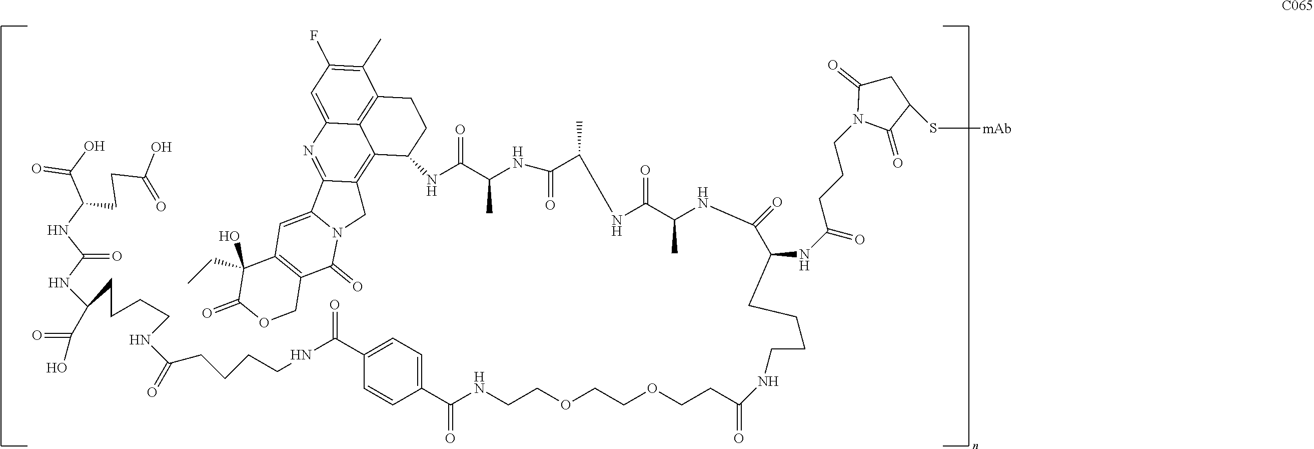 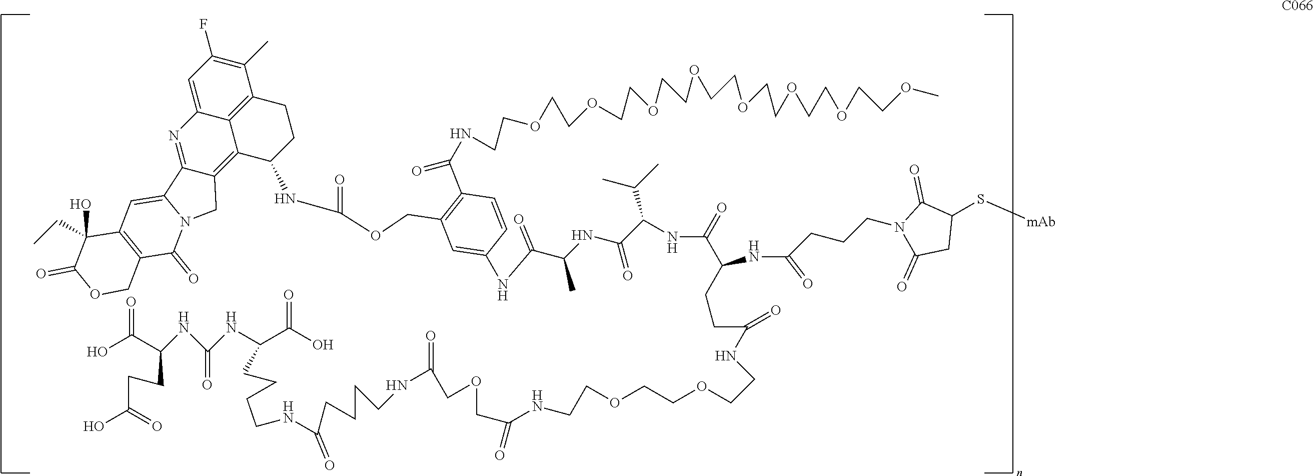 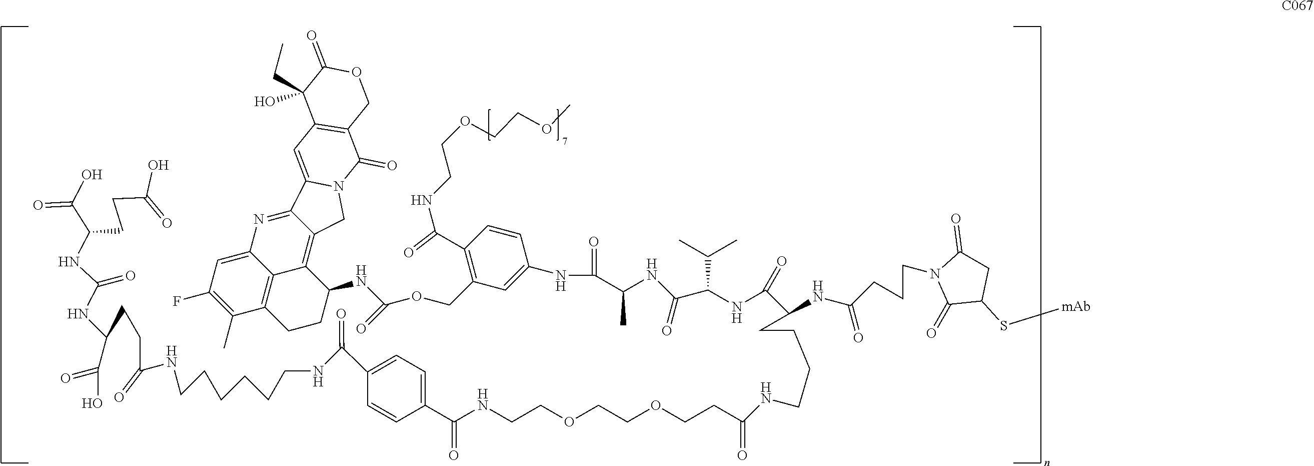 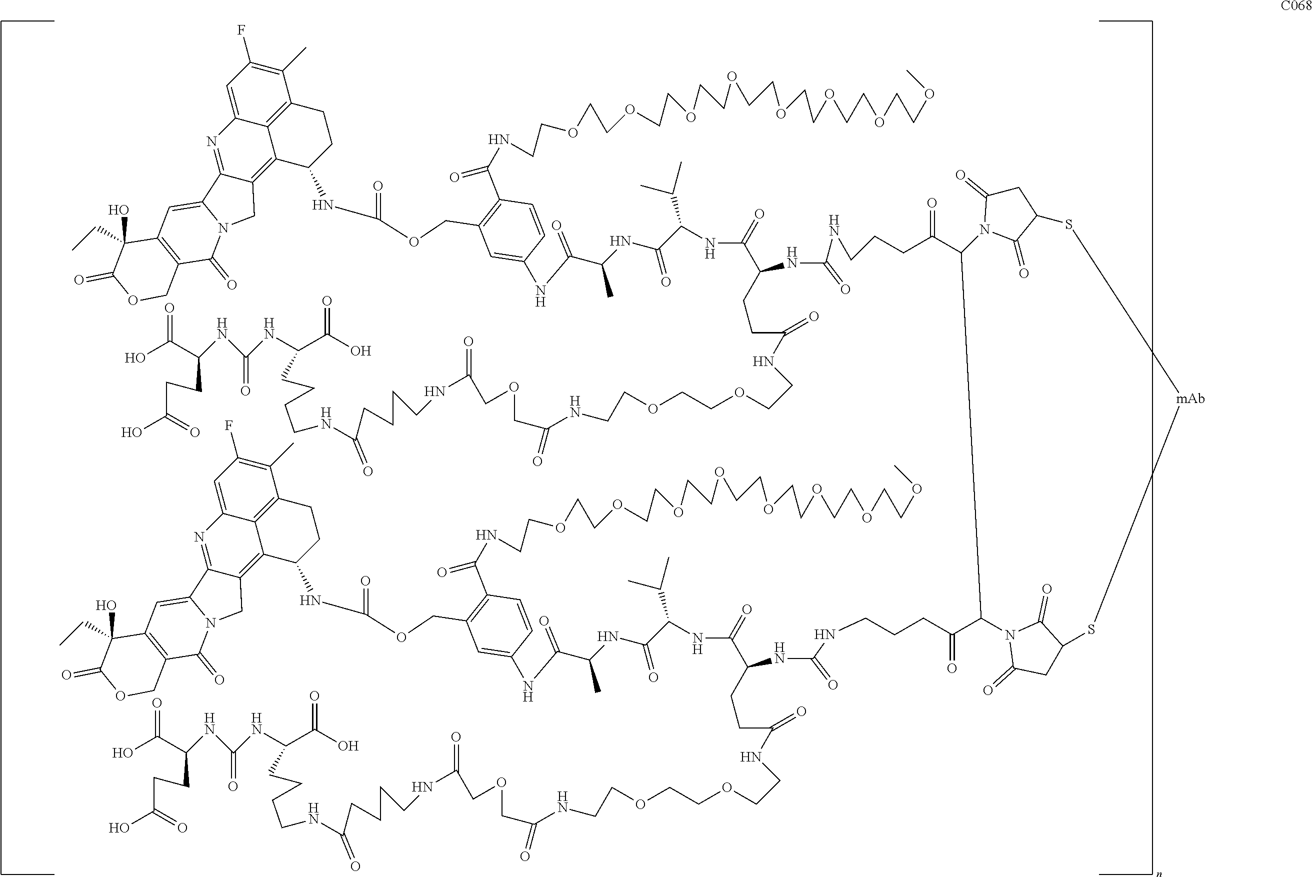 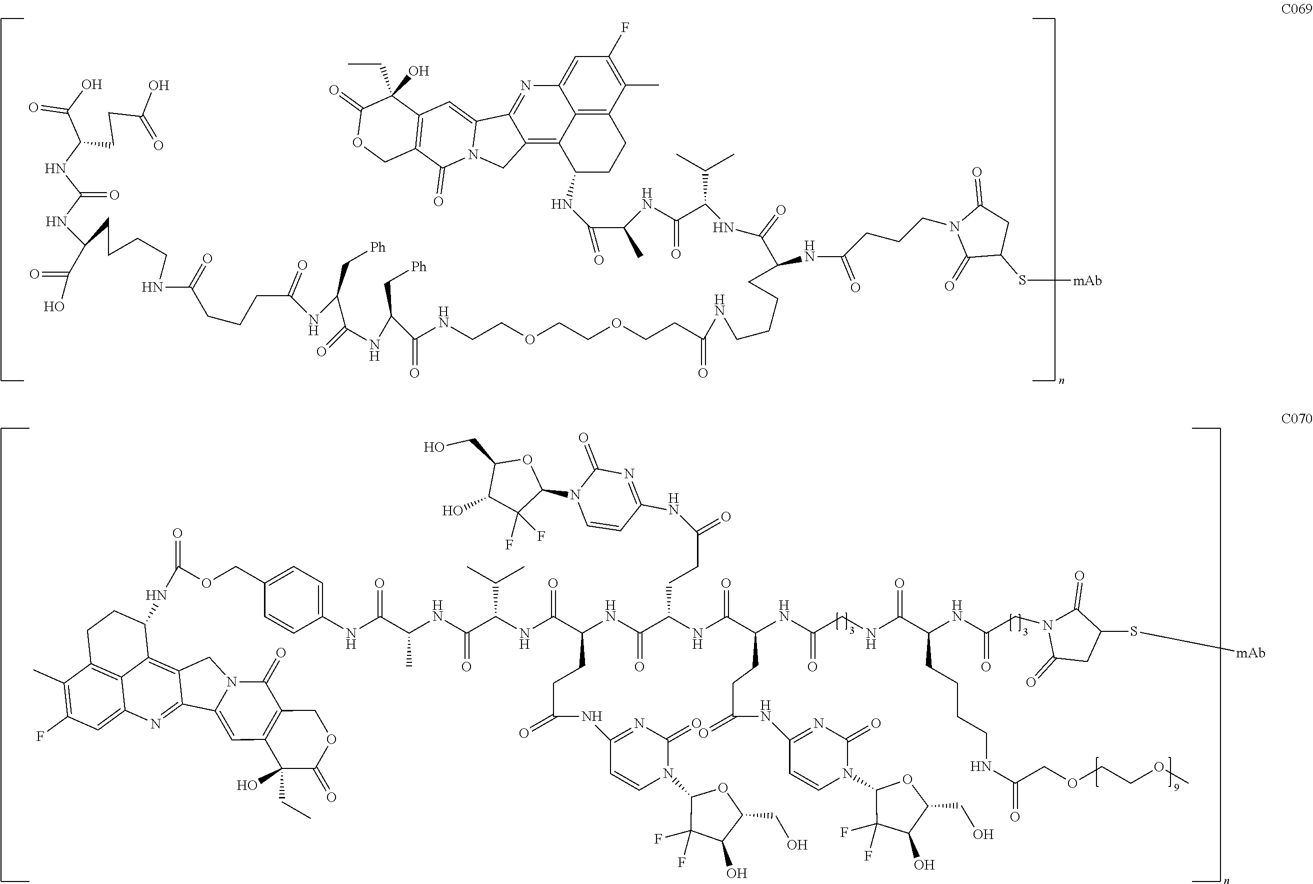 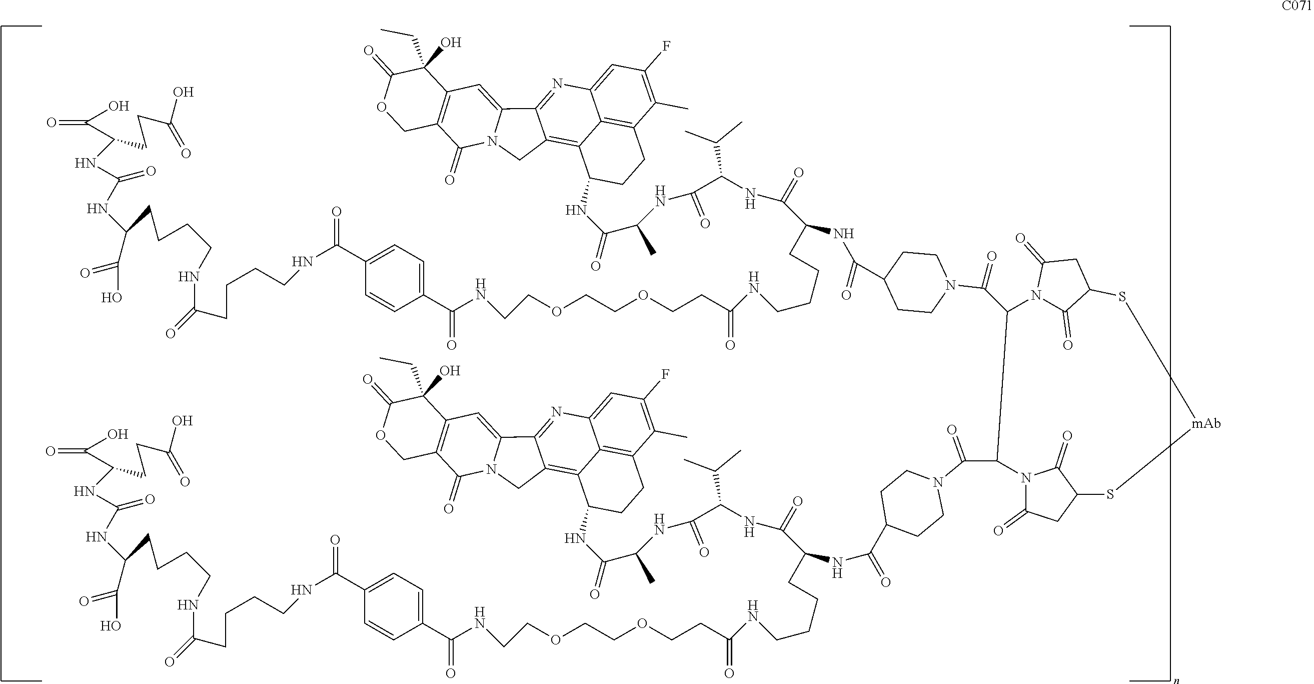 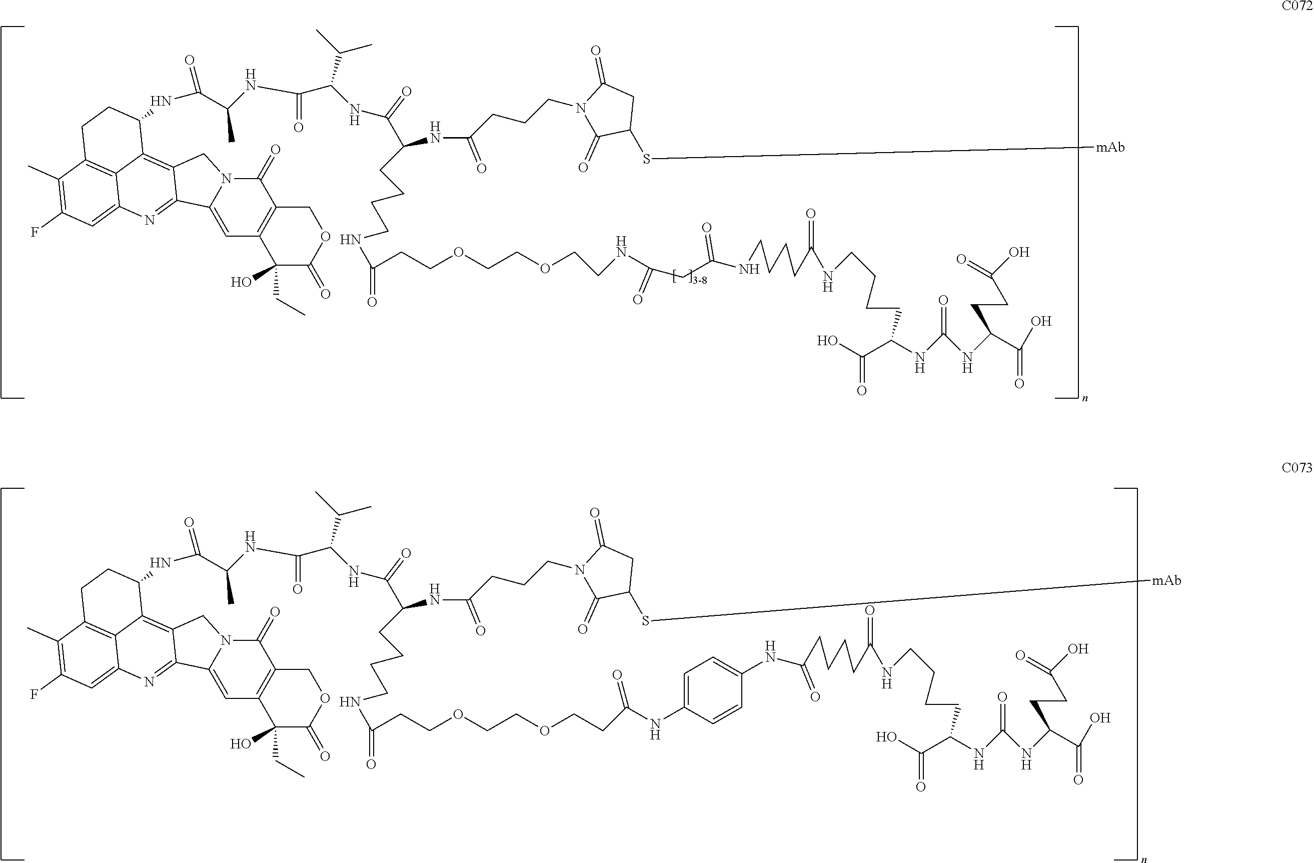 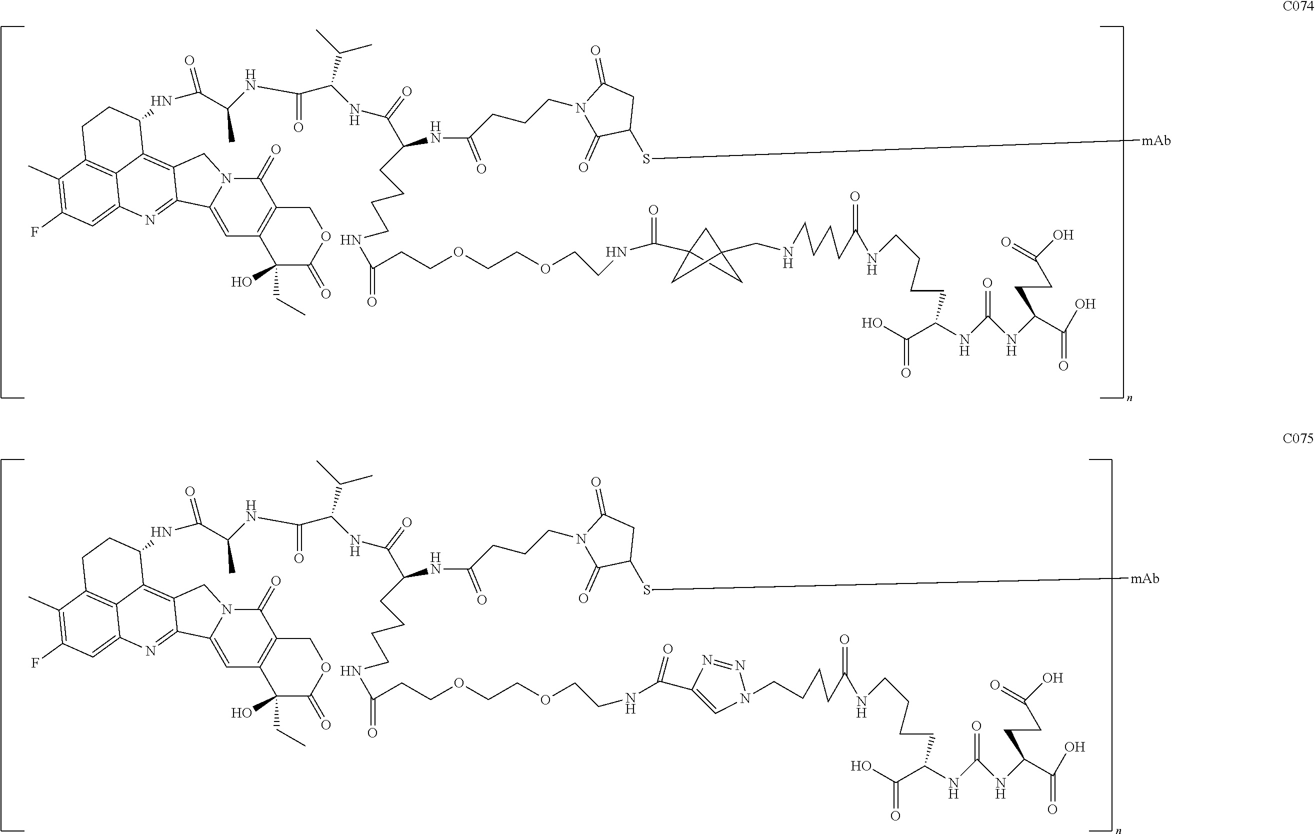 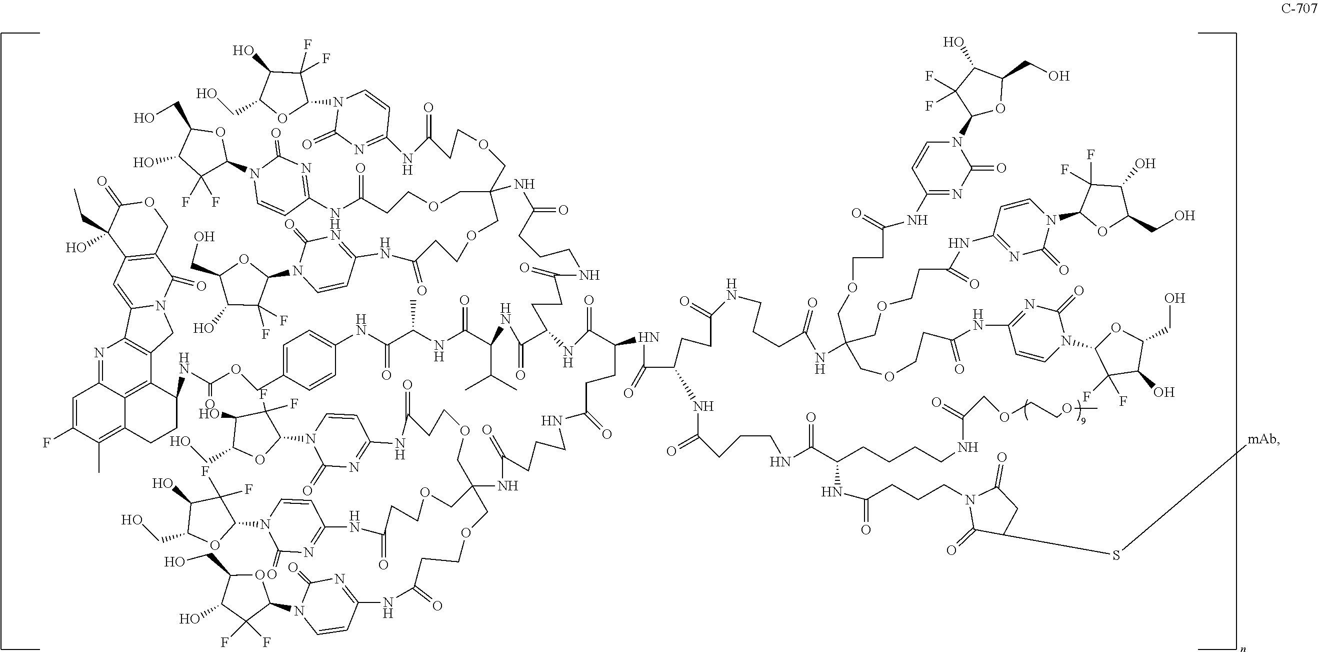 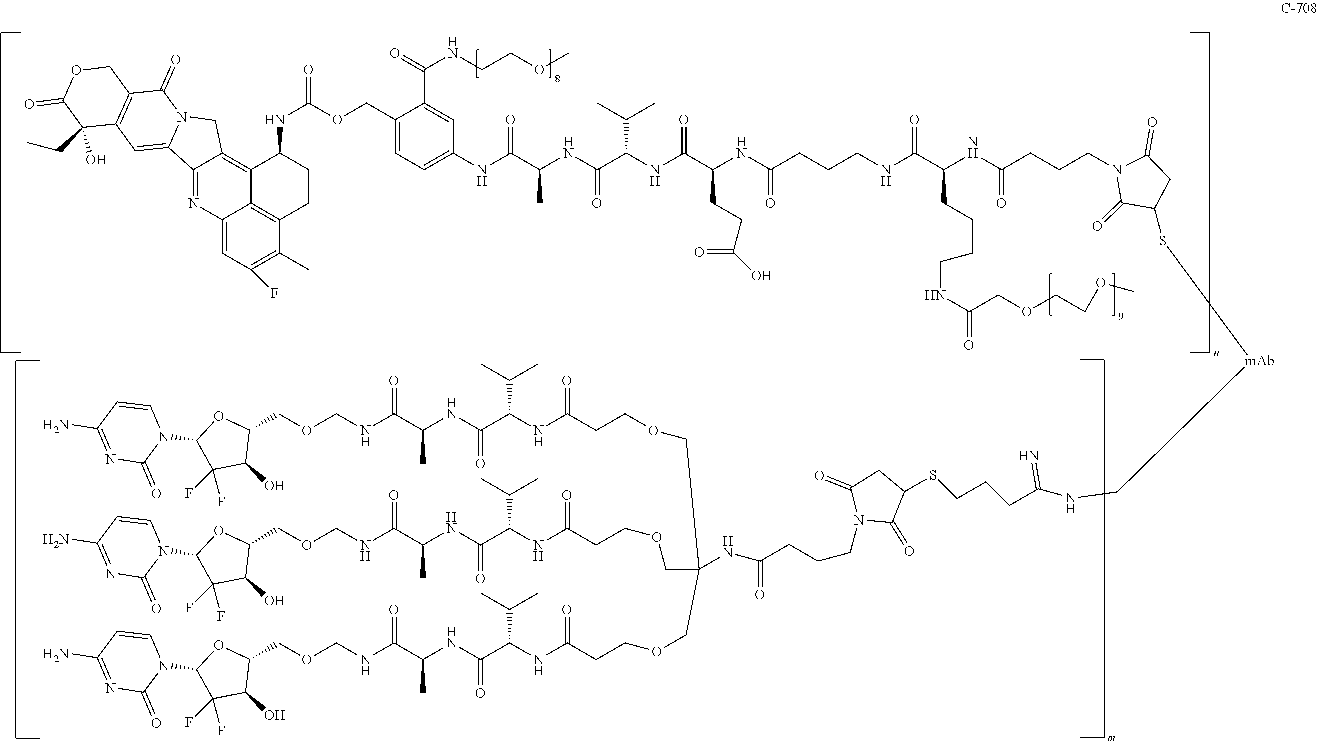 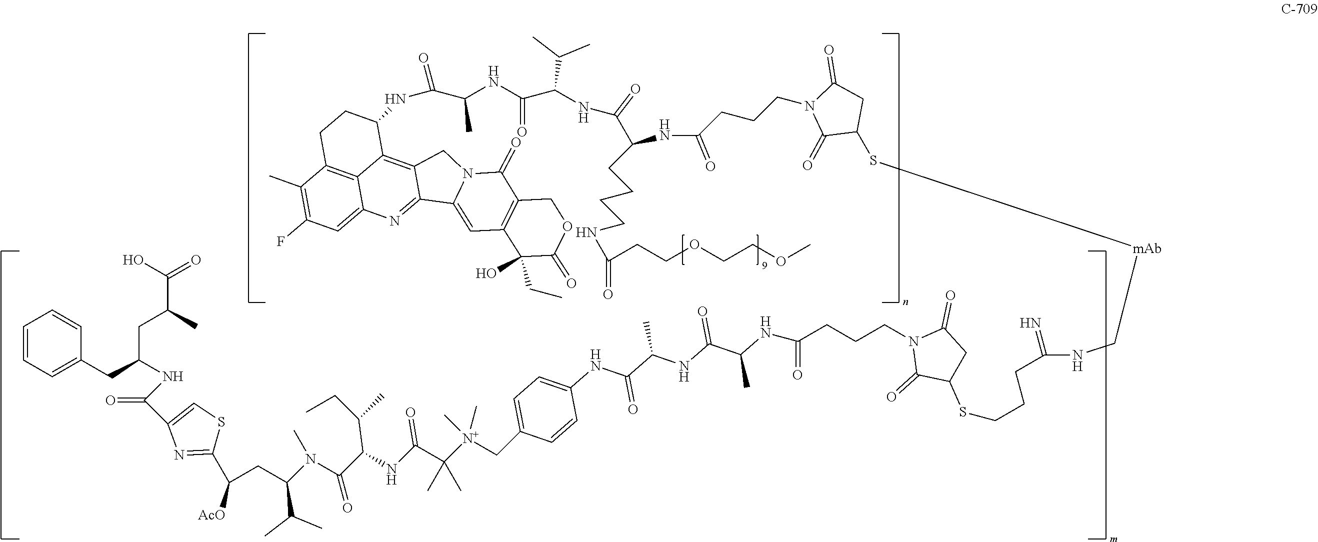 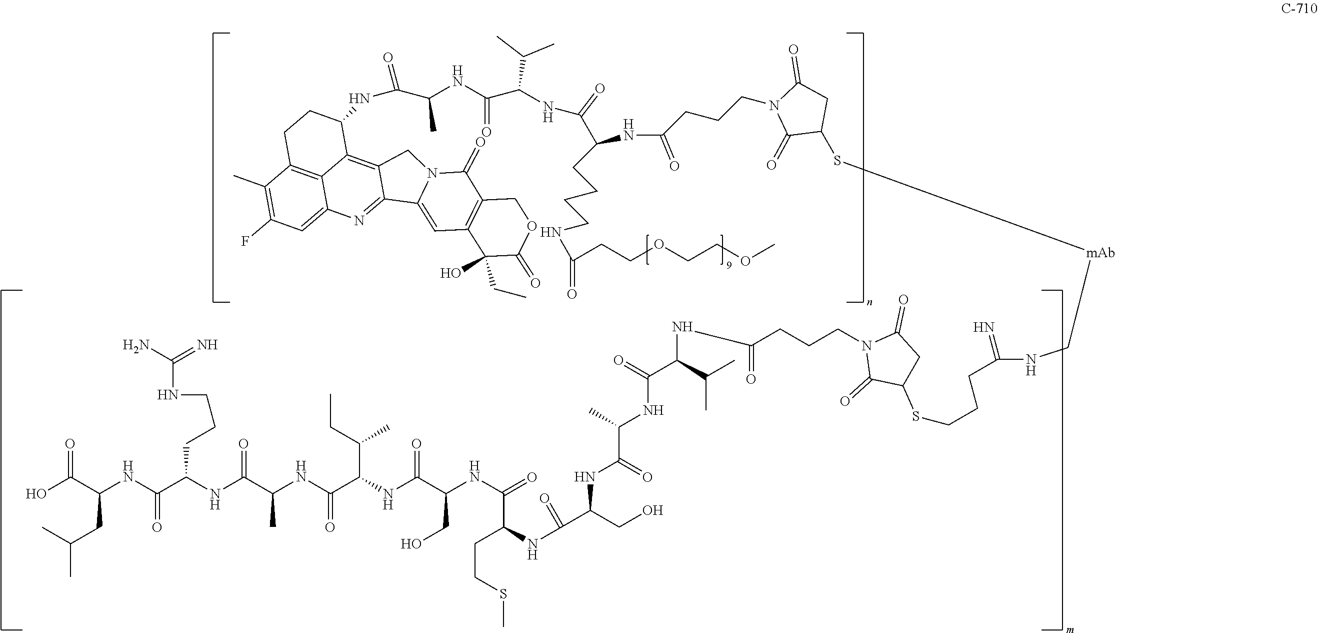 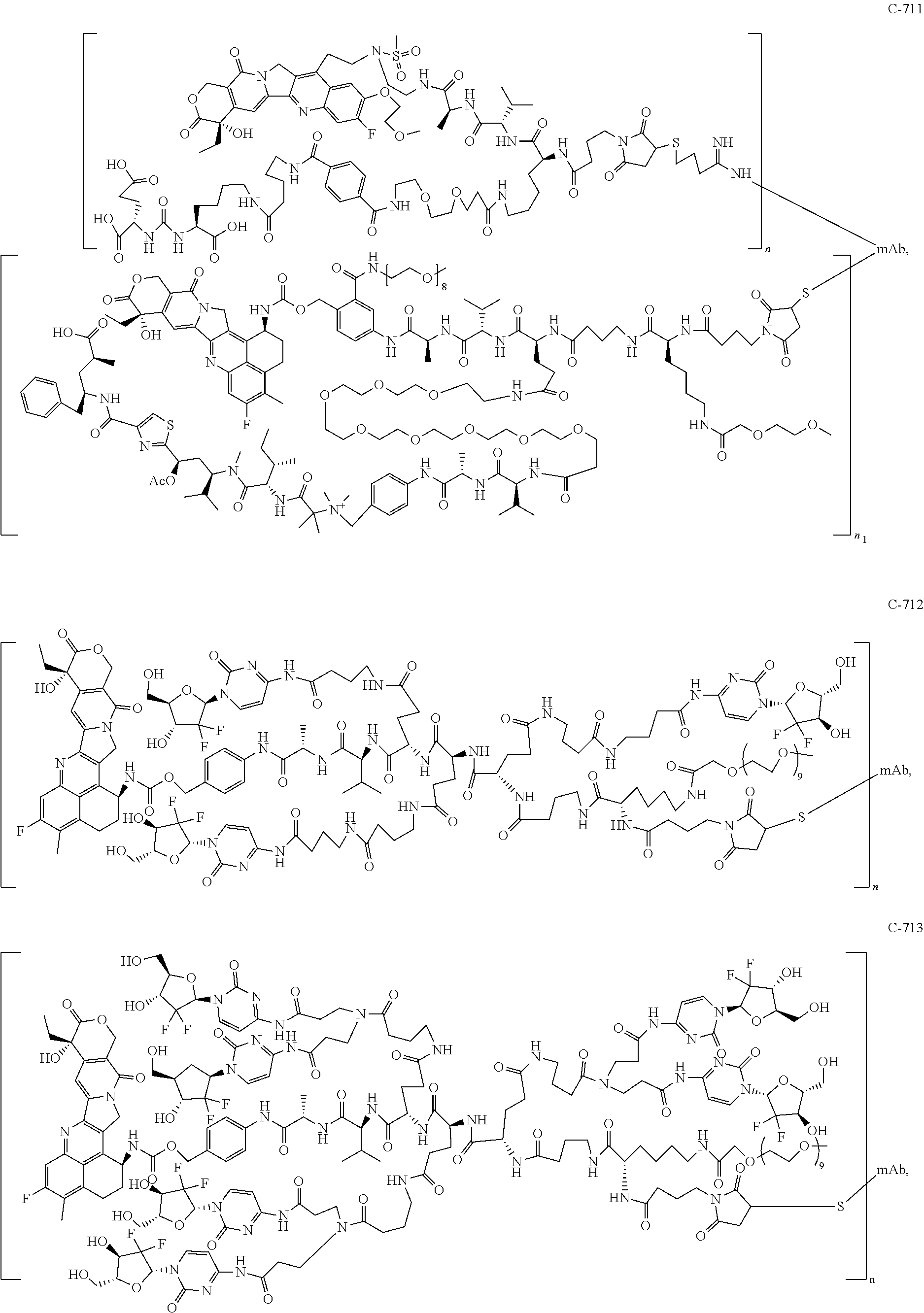
wherein mAb above is an antibody; n and n1 are 1˜30.

41. The antibody drug conjugate according to claim 1, wherein the conjugates of Formula (I), (II), (III) and (IV) are prepared via conjugation reaction of the antibody with a compound having the following Formula (V), (VI) and (VII) respectively: 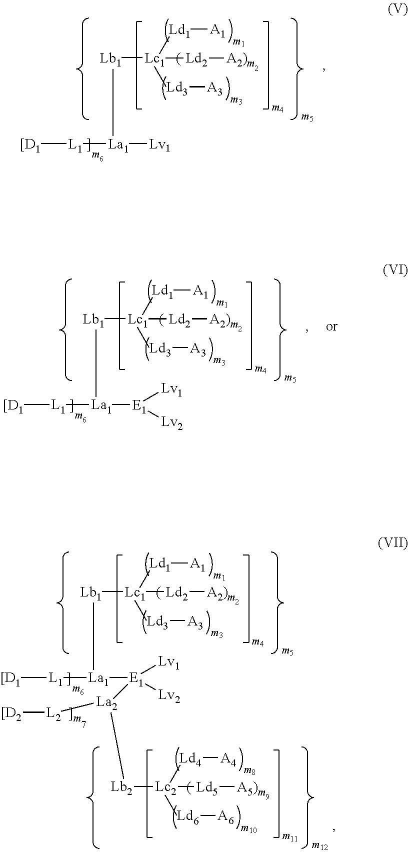
or the conjugate of Formula (IV) is prepared through sequential conjugation reaction of Formula (V) and Formula (V′) to an antibody: 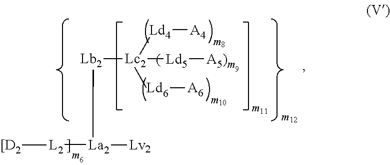
wherein D₁, D₂, L₁, L₂, La₁, La₂, Lb₁, Lb₂, Lc₁, Lc₂, Ld₁, Ld₂, Ld₃, Ld₄, Ld₅, Ld₆, A₁, A₂, A₃, A₄, A₅, A₆, E₁, m1, m2, m3, m4, m5, m6, m7, m8, m9, m10, m11, and m12 are defined the same in the claim 1; Lv₁ and Lv₂ are a reactive group and are independently or fused jointly selected from: 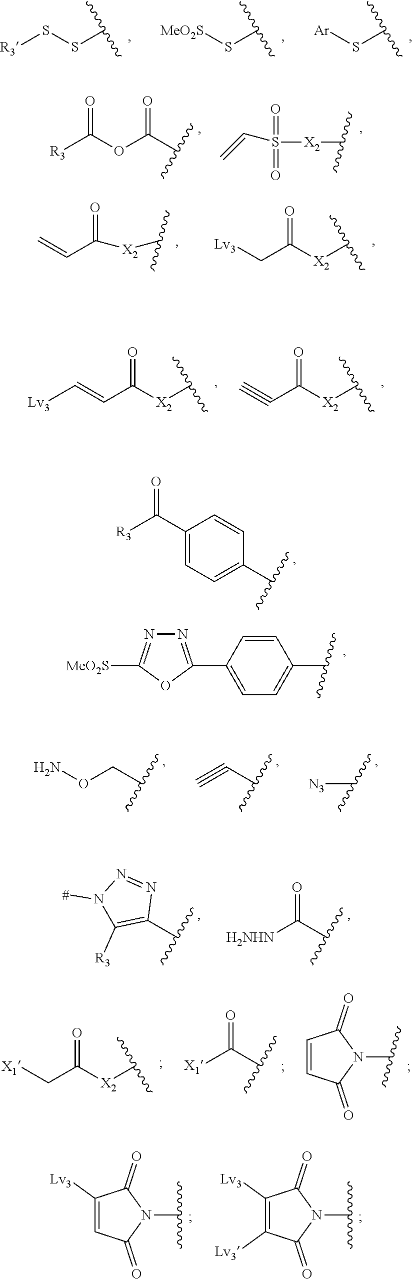 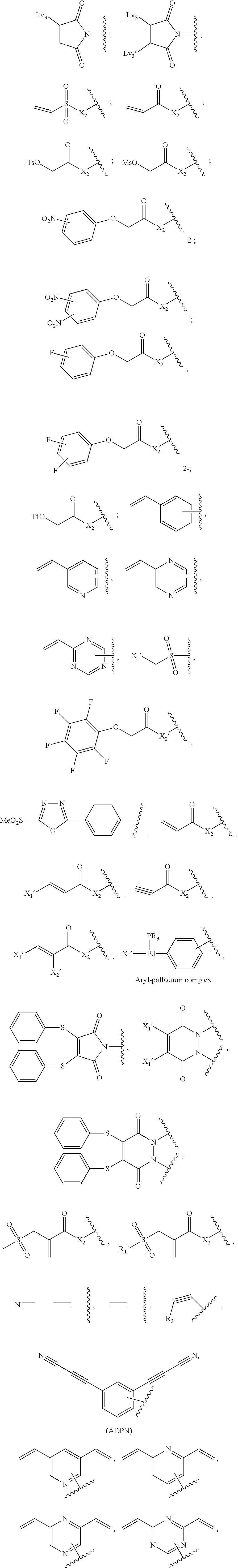 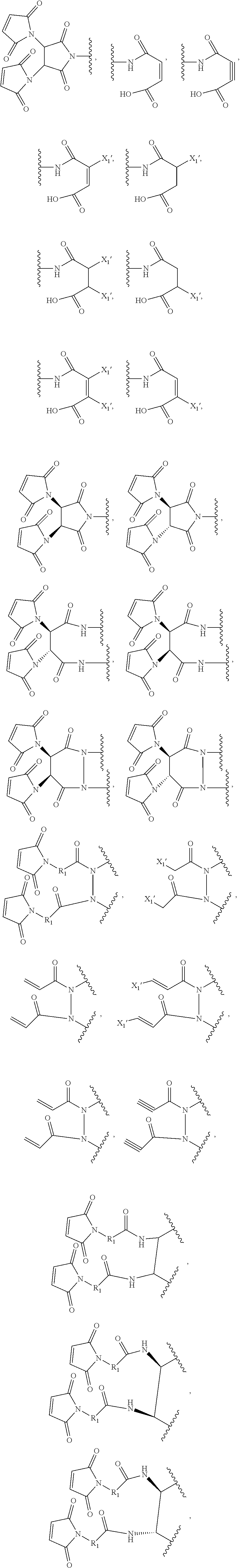 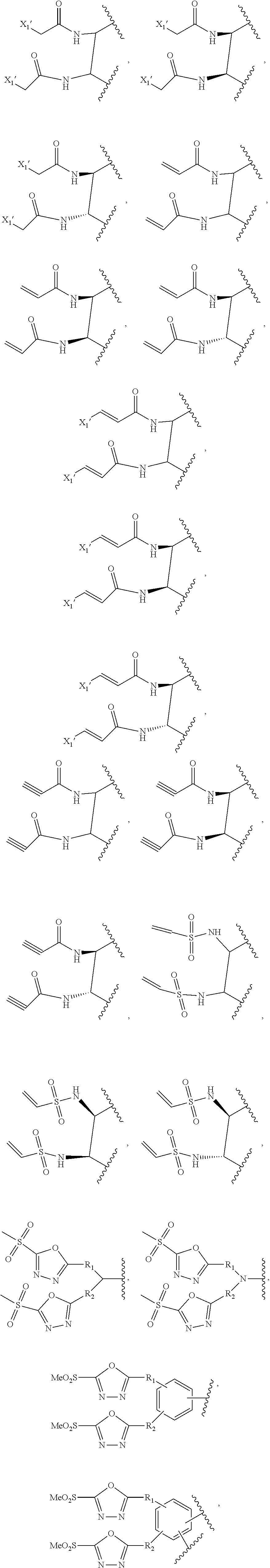 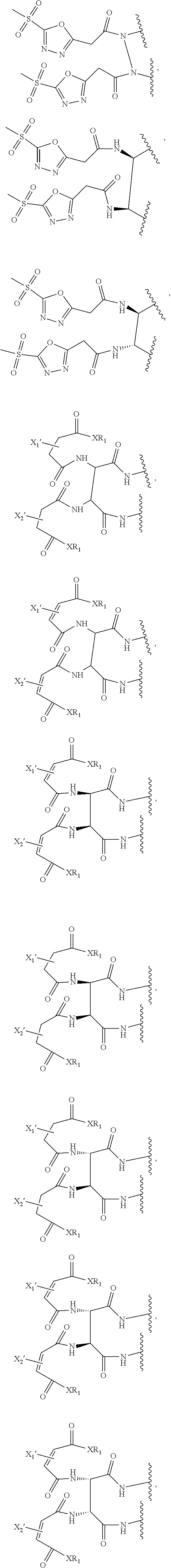 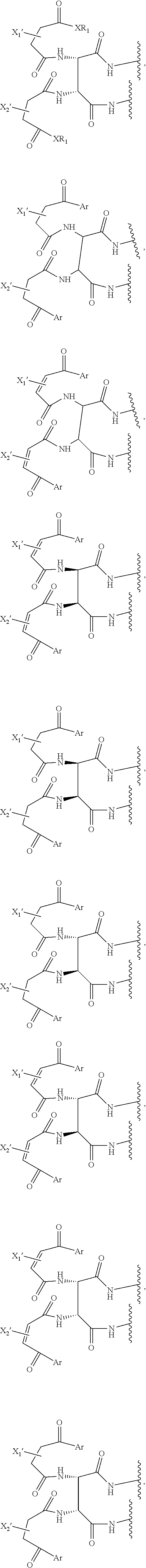 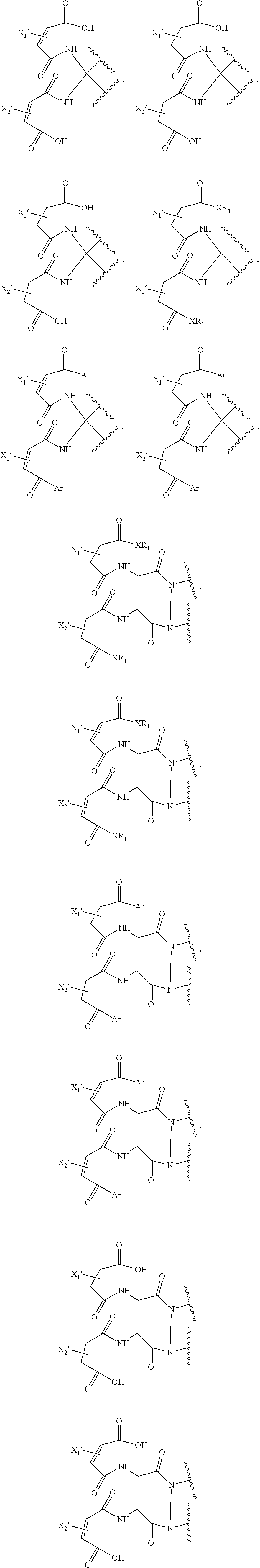 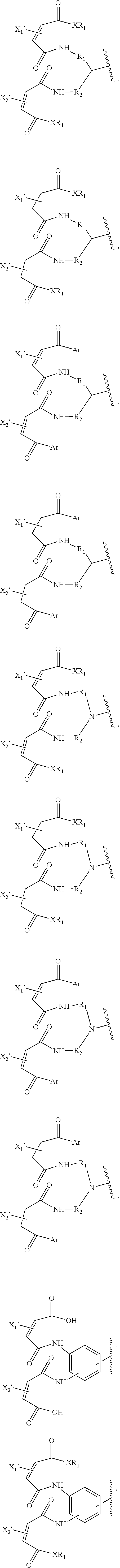 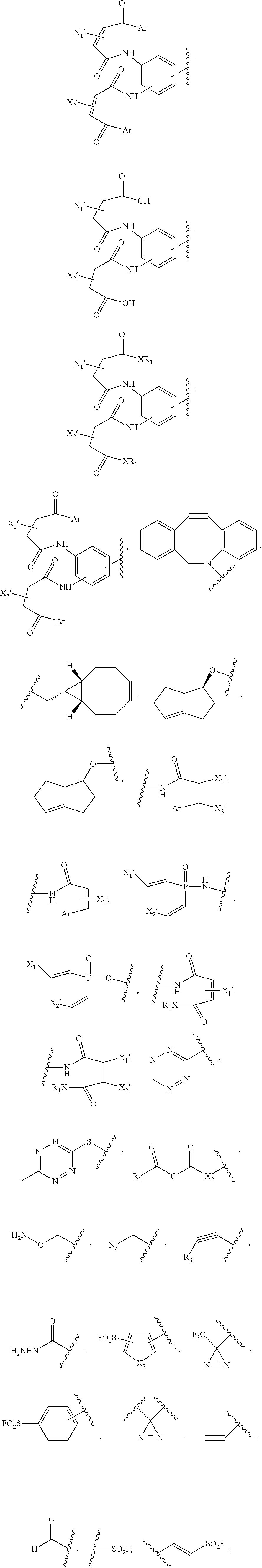
wherein X₁′ and X₂′ are independently F, Cl, Br, I, OTf, OMs, OC₆H₄(NO₂), OC₆H₃(NO₂)₂, OC₆F₅, OC₆HF₄, or Lv₃; X₂ is O, NH, N(R₁), or CH₂; R₃ and R₅ are independently H, R₁, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ and Lv₃′ are independently a leaving group selected from F, Cl, Br, I, nitrophenol; N-hydroxysuccinimide (NHS); phenol; benzenethiol, dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

42. The antibody drug conjugate according to claim 41, wherein in the compound in Formula (VI) and (VII), the joint structure of 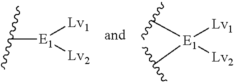
are accordingly selected from: 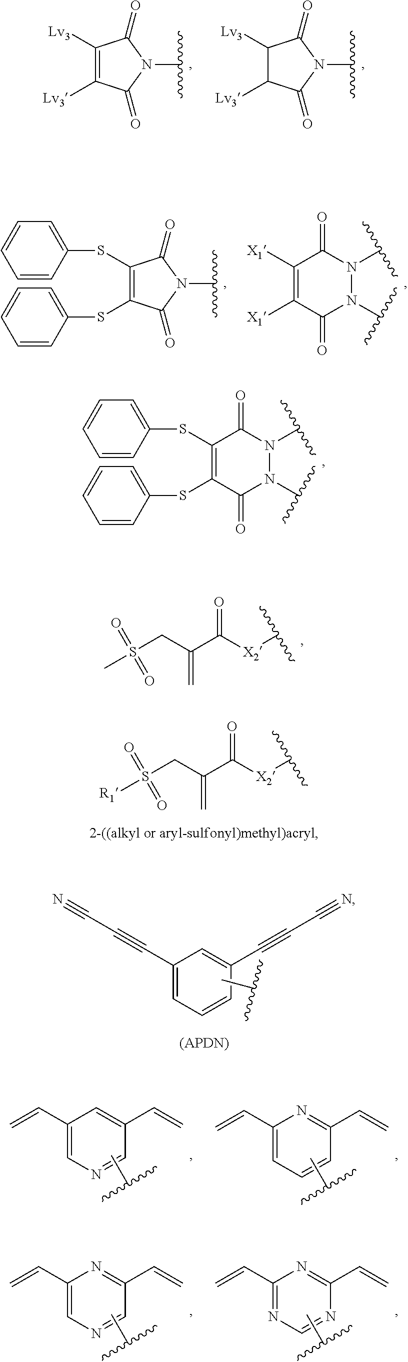 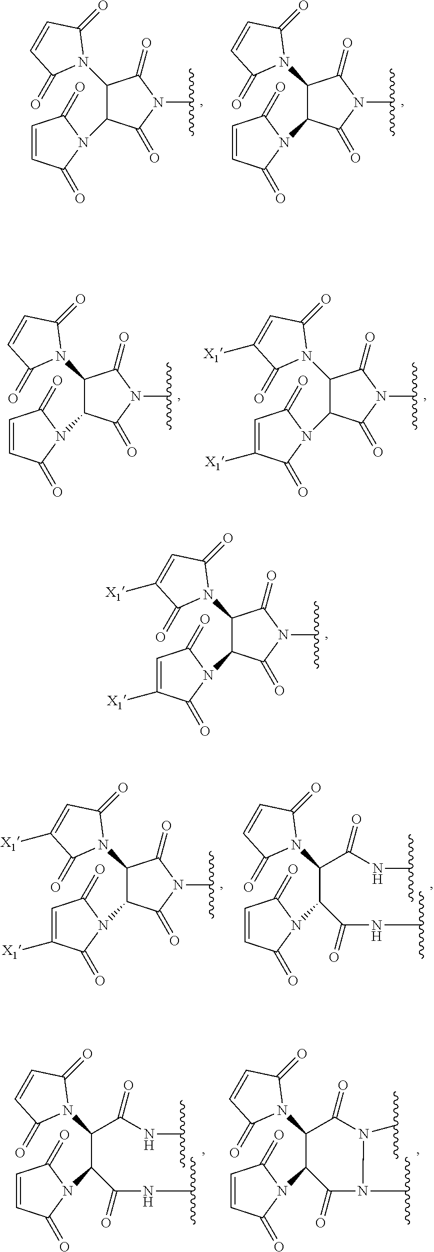 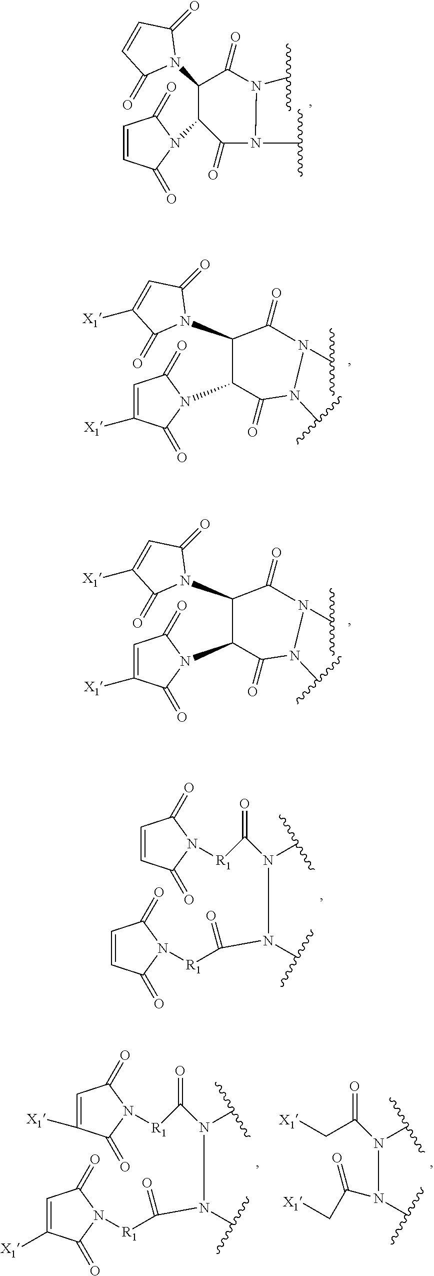 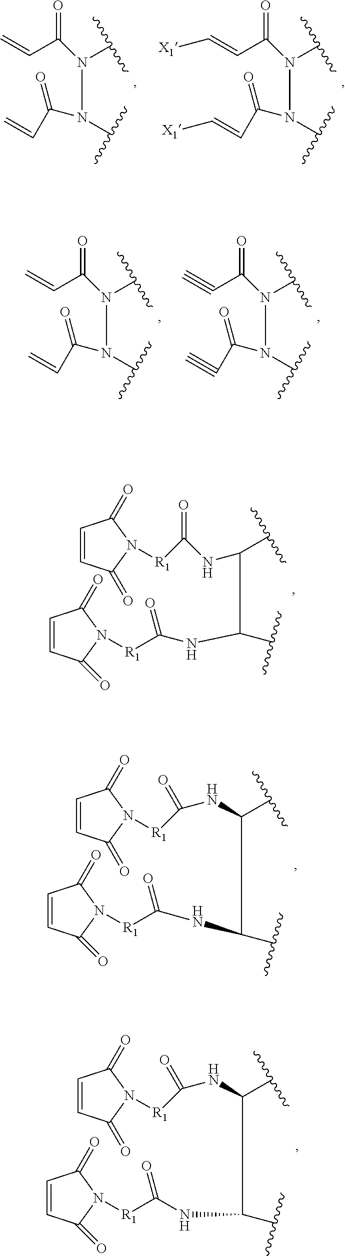 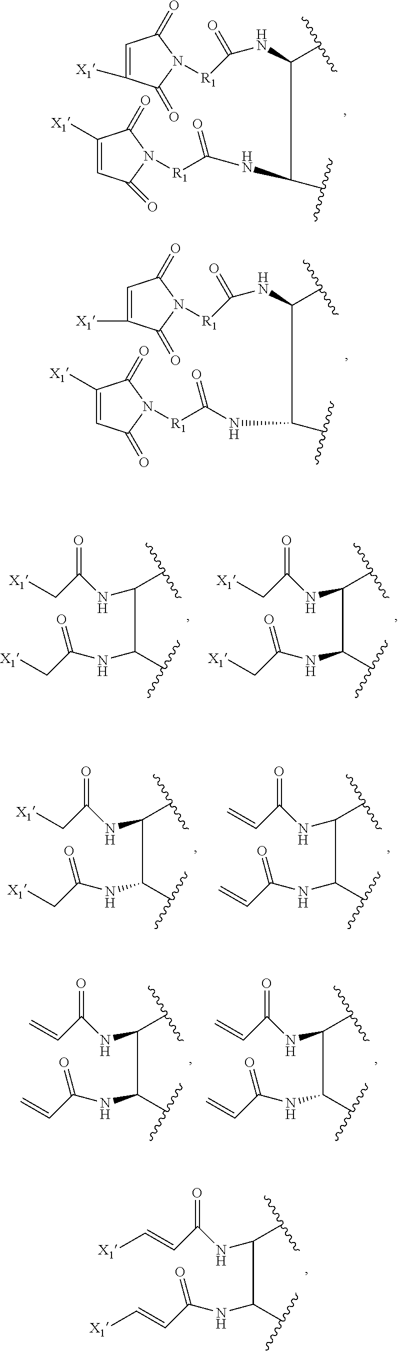 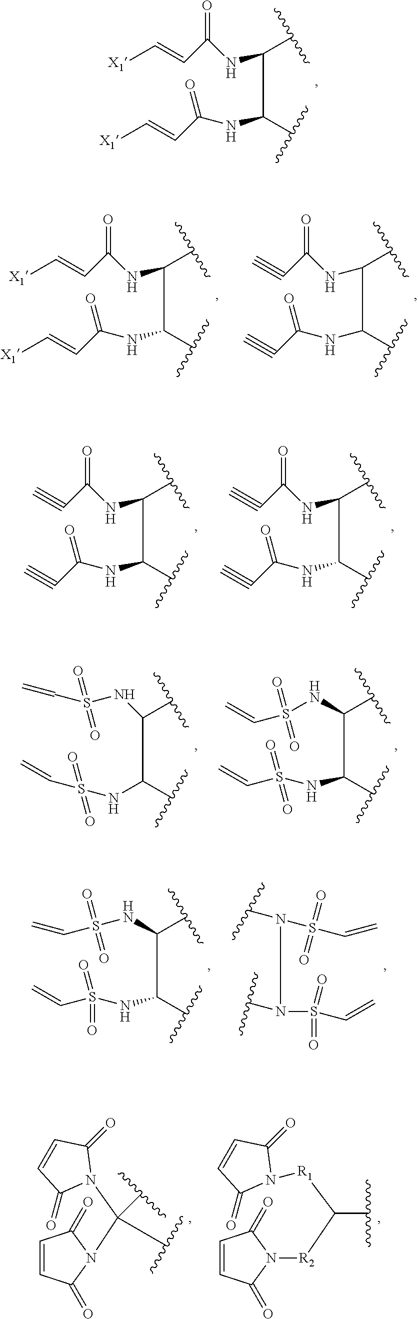 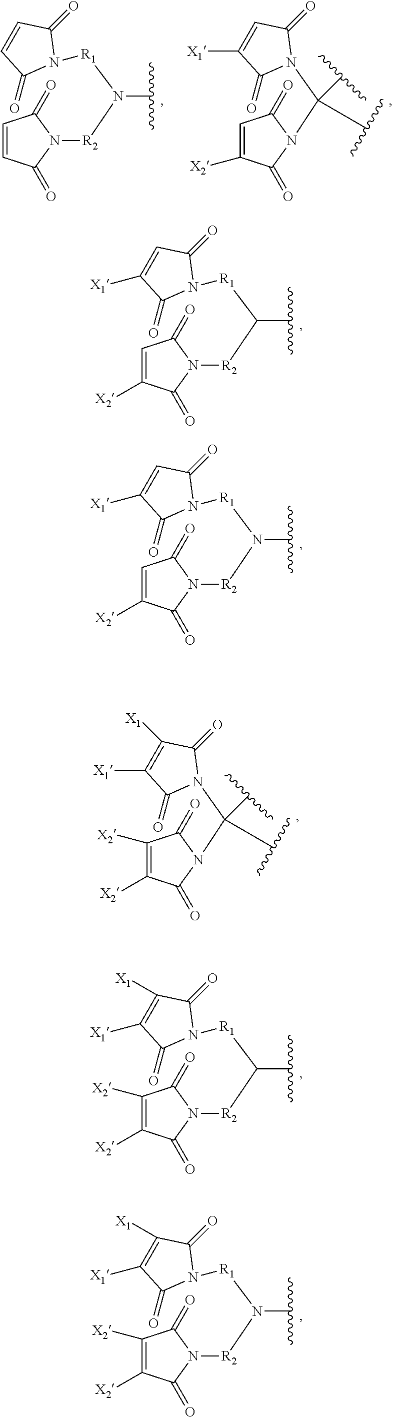 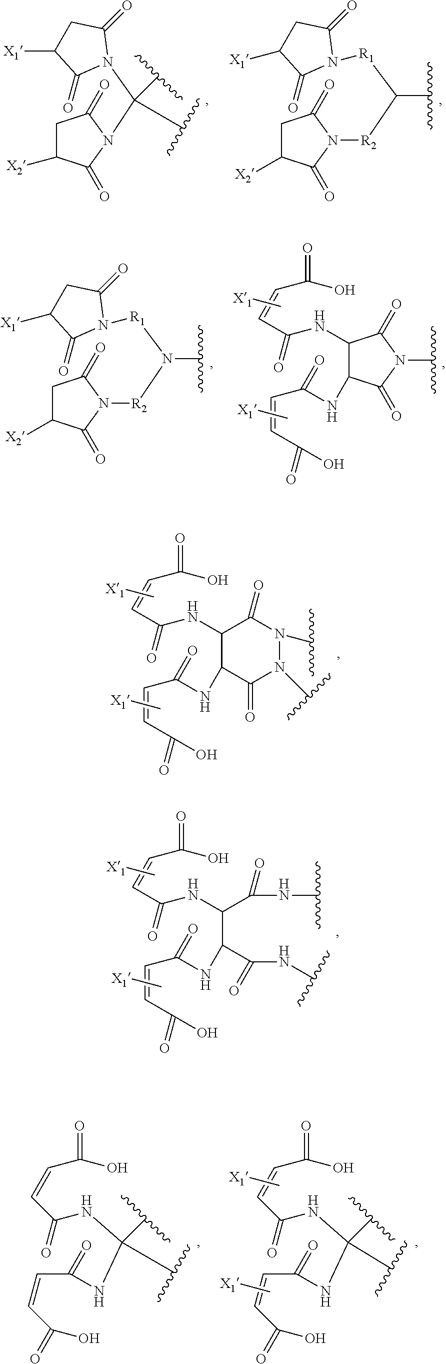 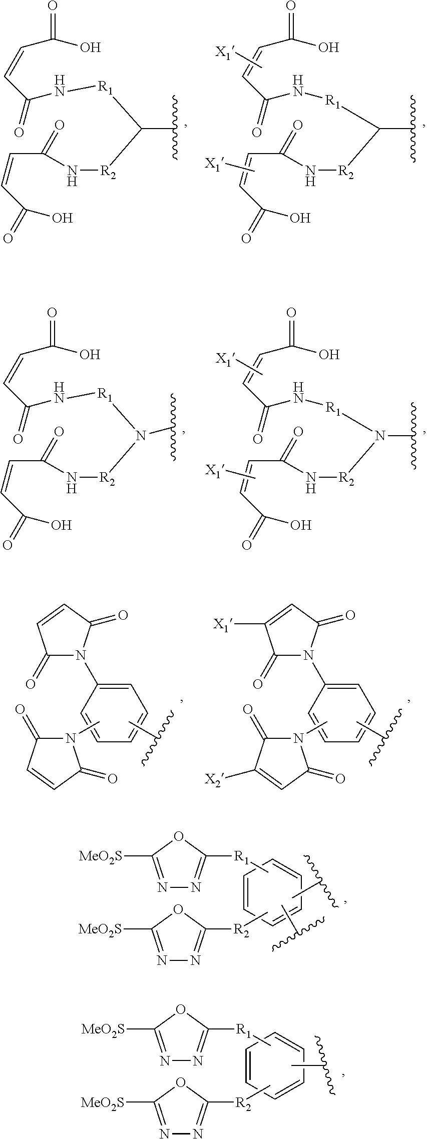 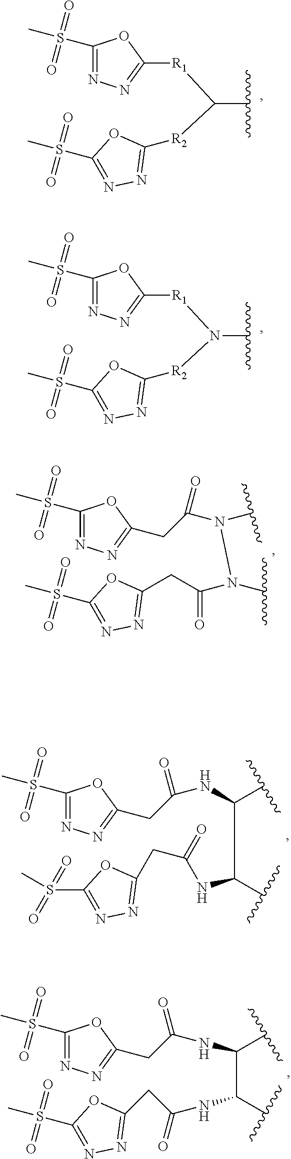 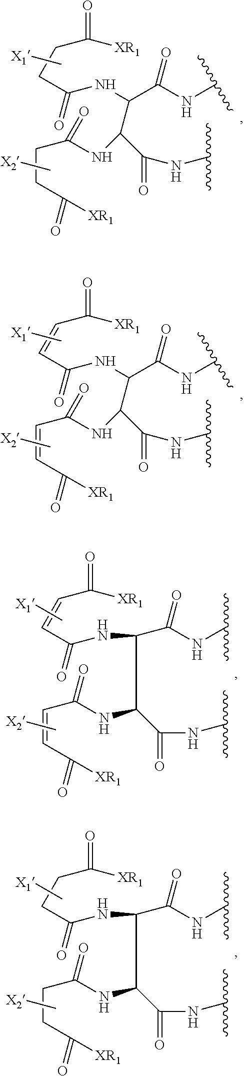 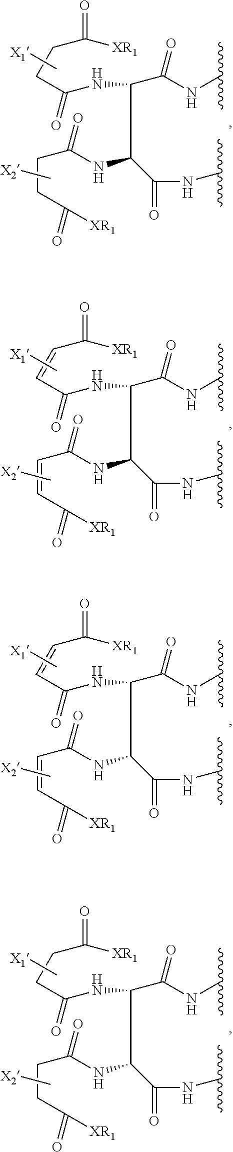 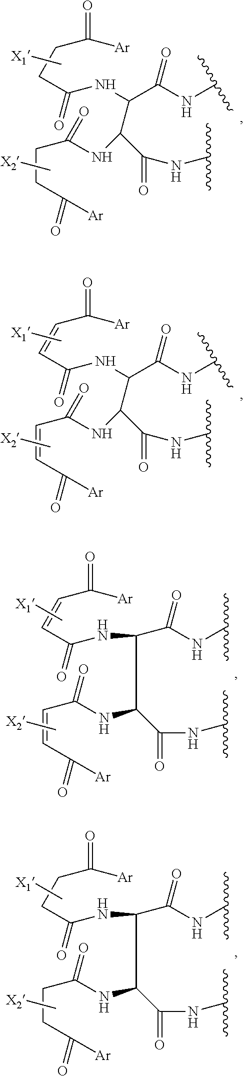 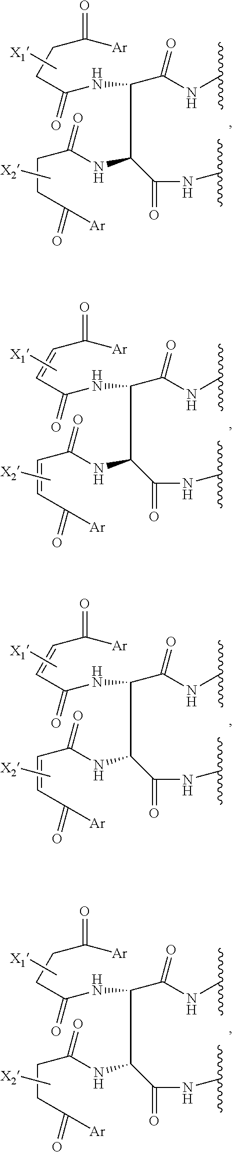 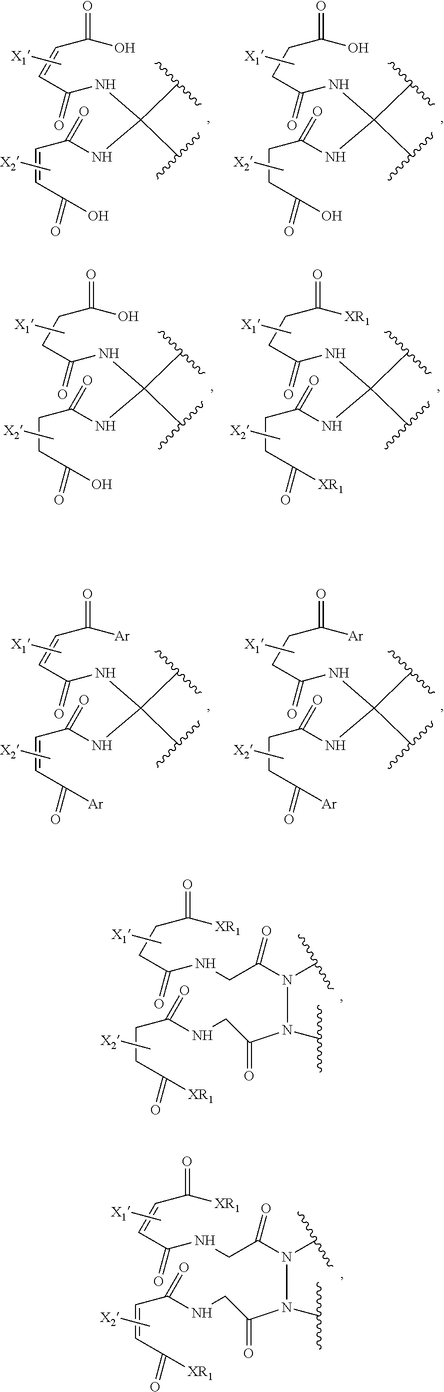 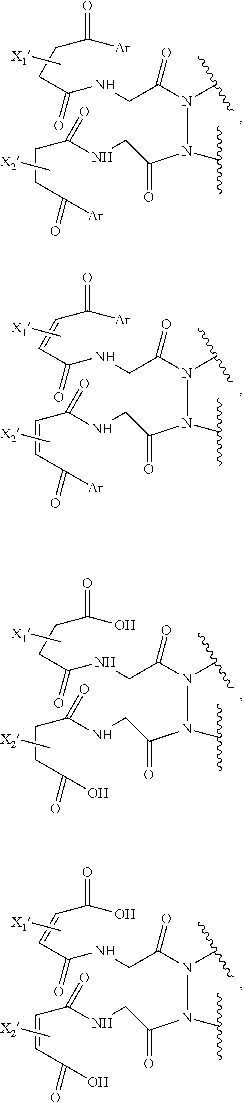 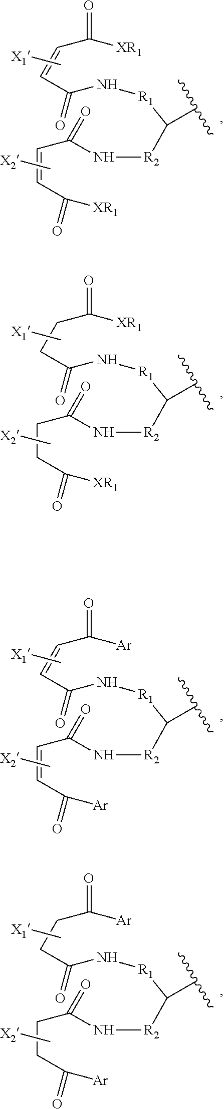 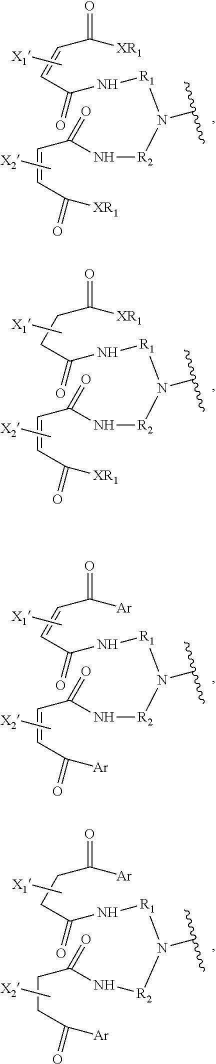 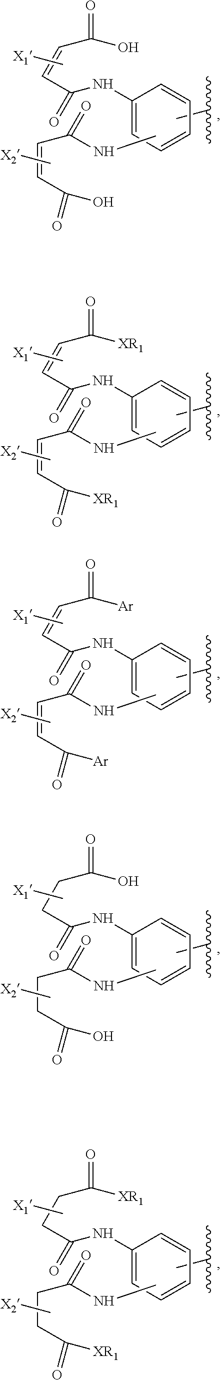 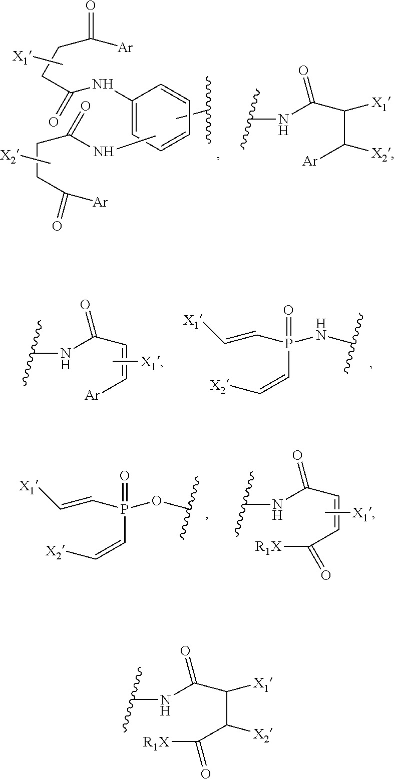
wherein Lv₃, Lv₃40, X₁40 0 and X₂40 are described above in claim 41; the connecting bond “—” in the middle of the two atoms means it can link either one of the two atoms.

43. The antibody drug conjugate according to claim 41, wherein the compound is represented as the following structures:

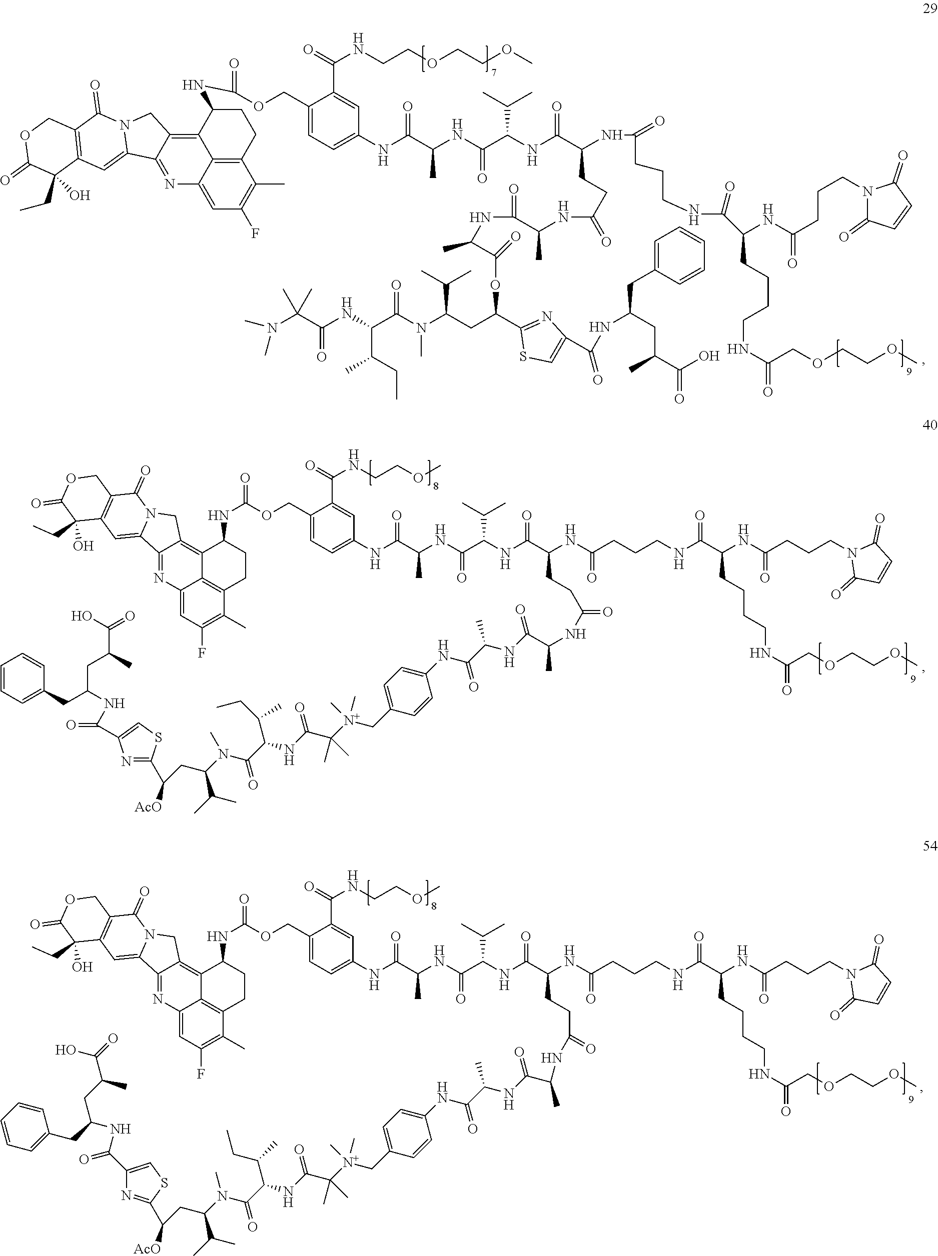

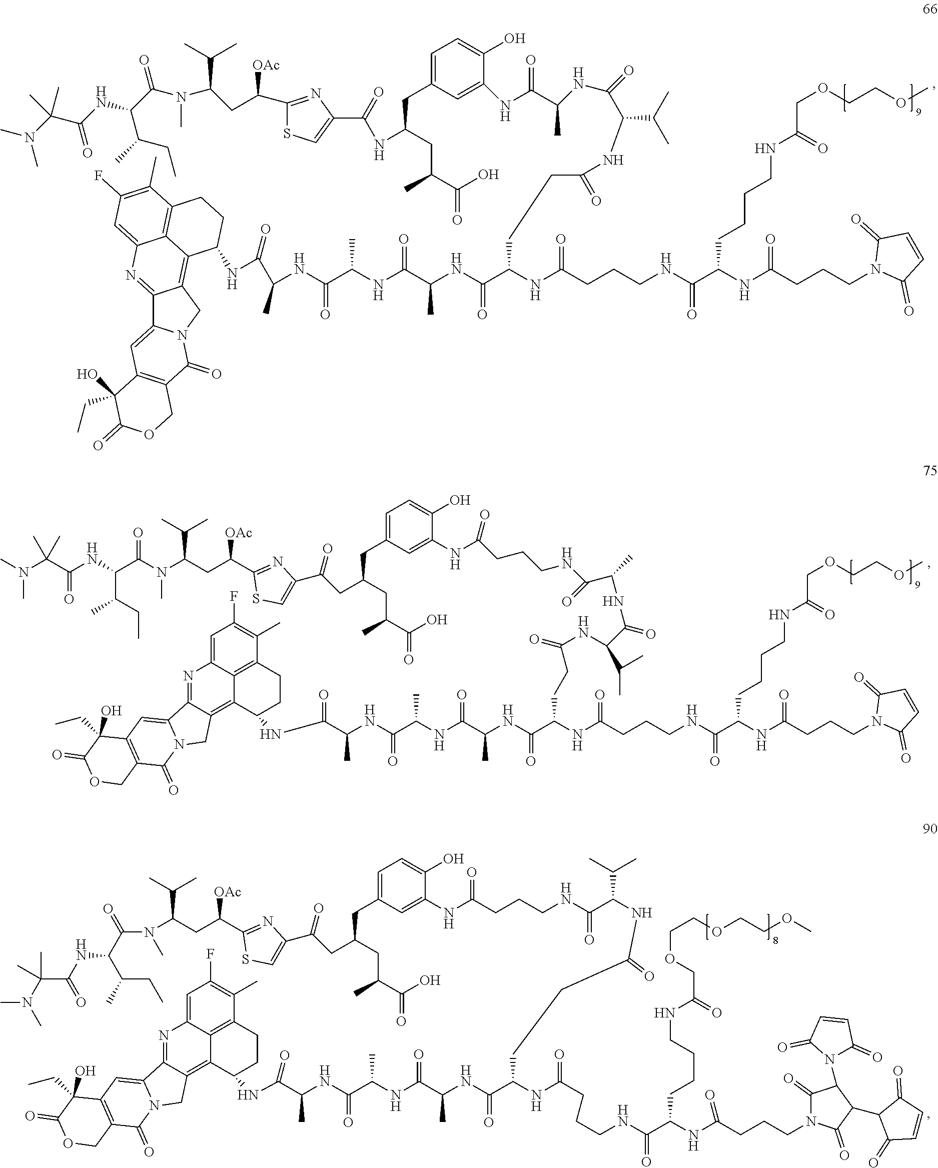

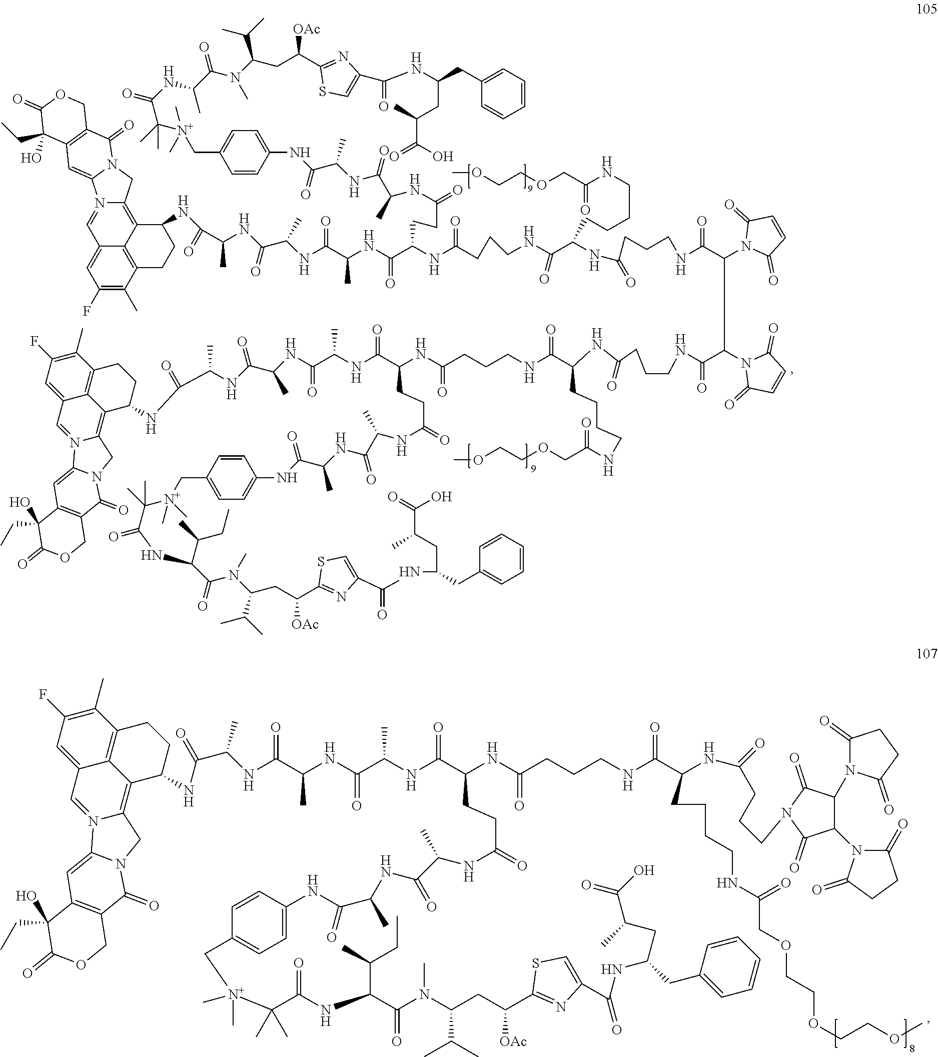

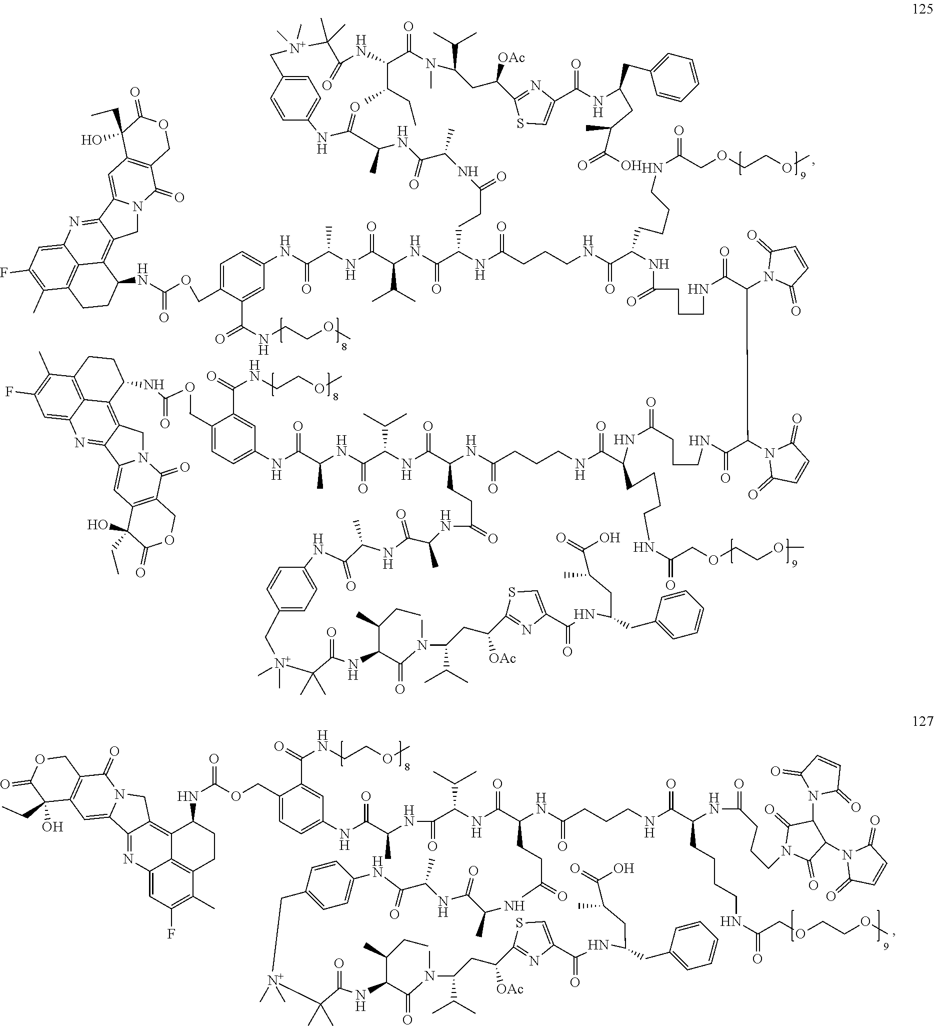

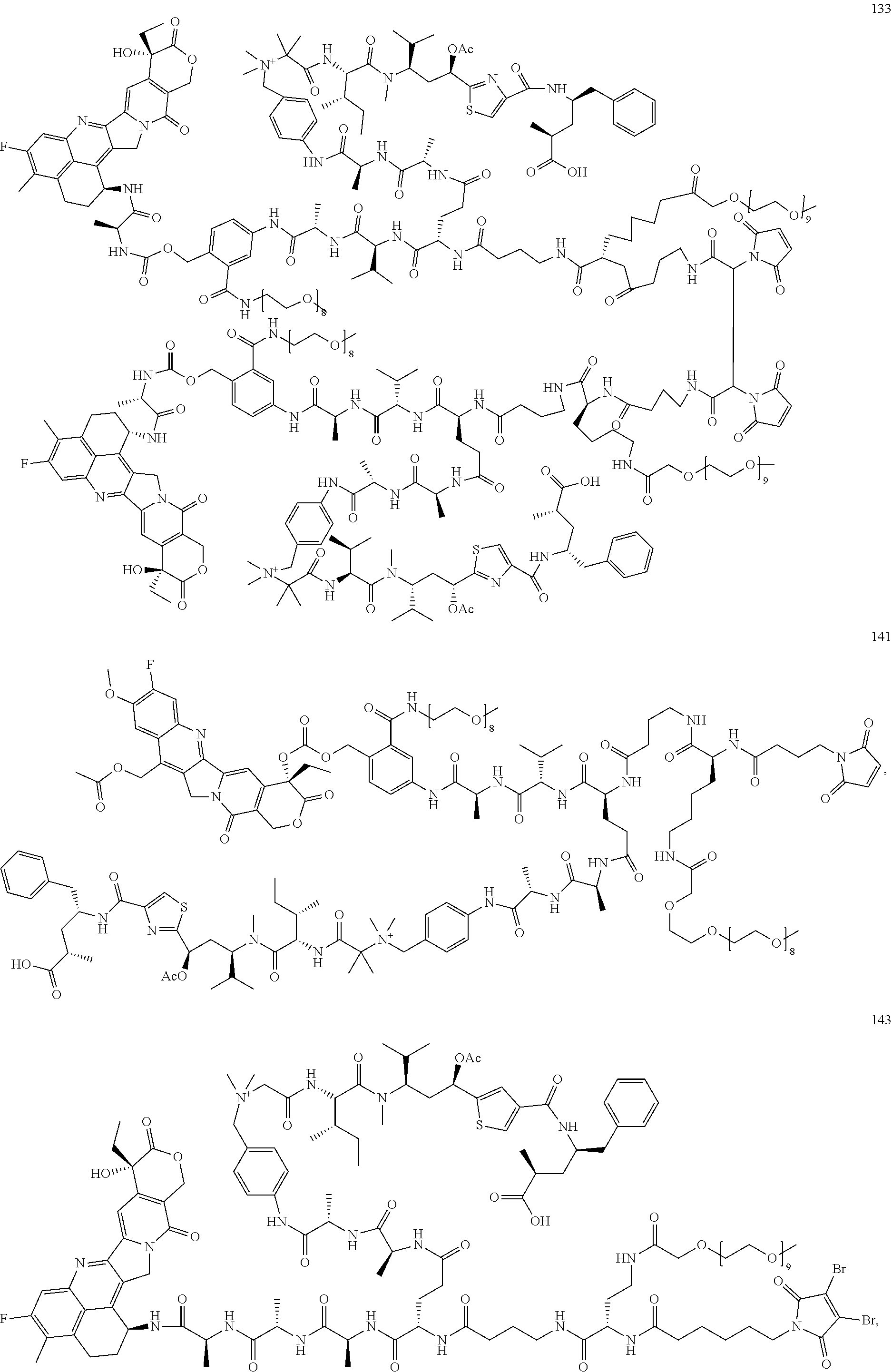

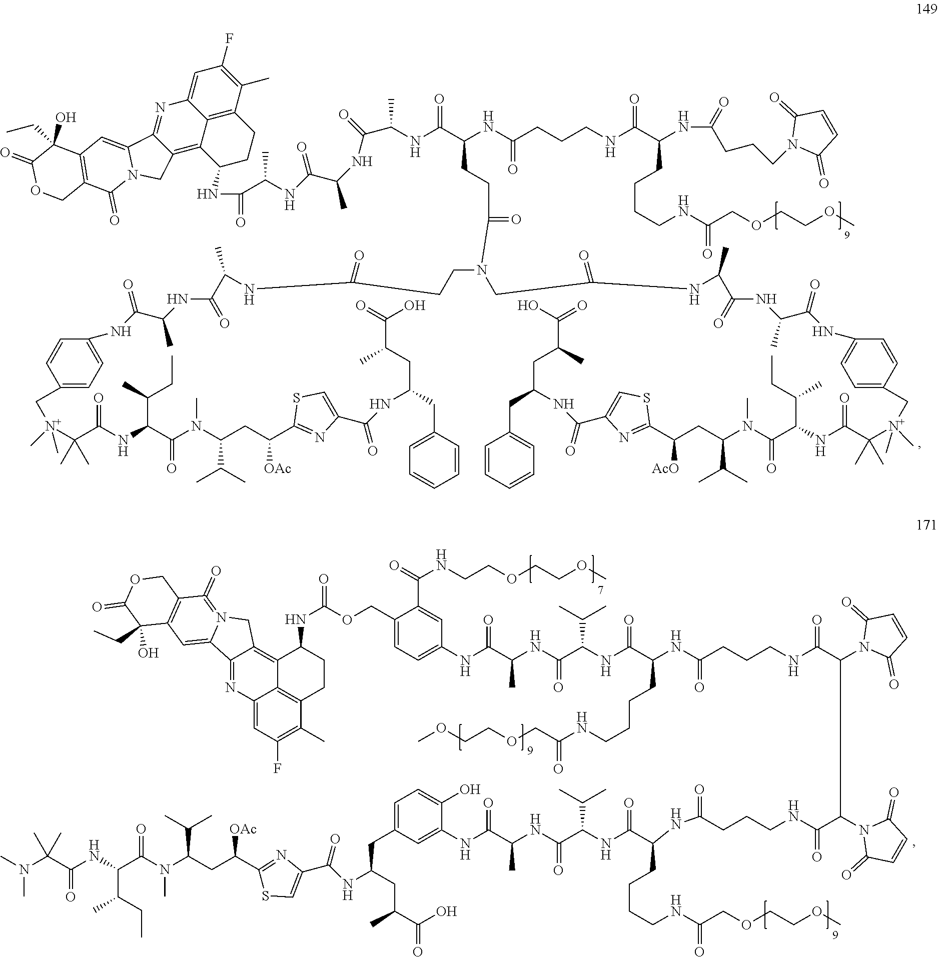

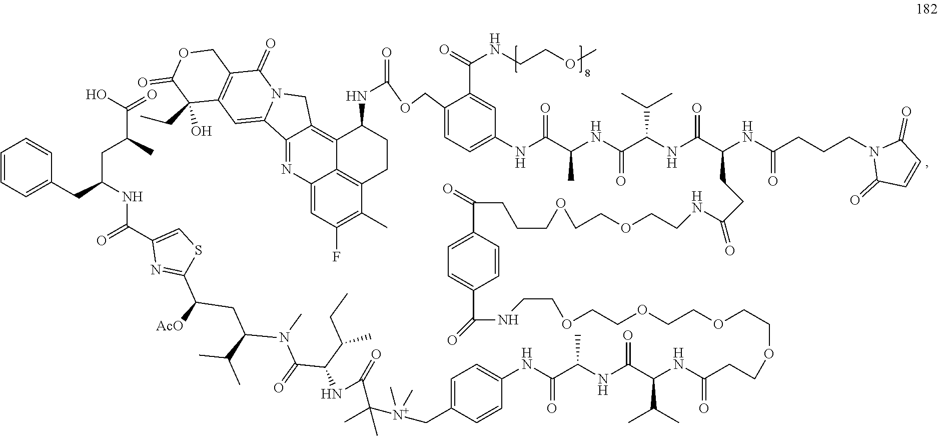

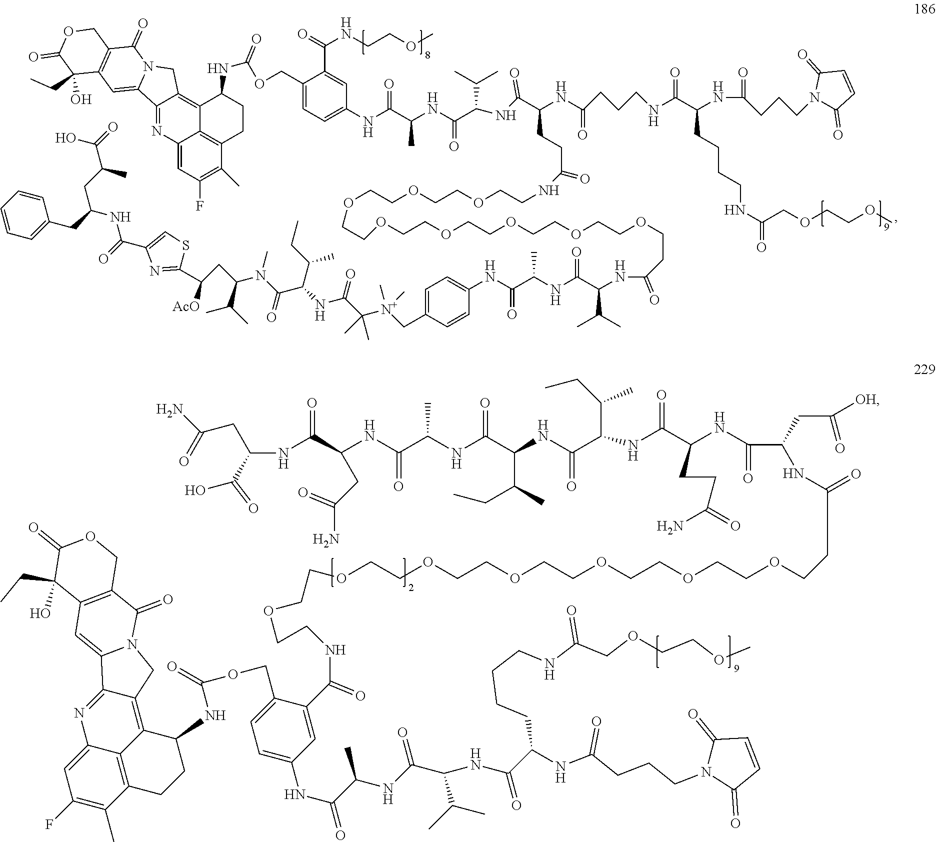

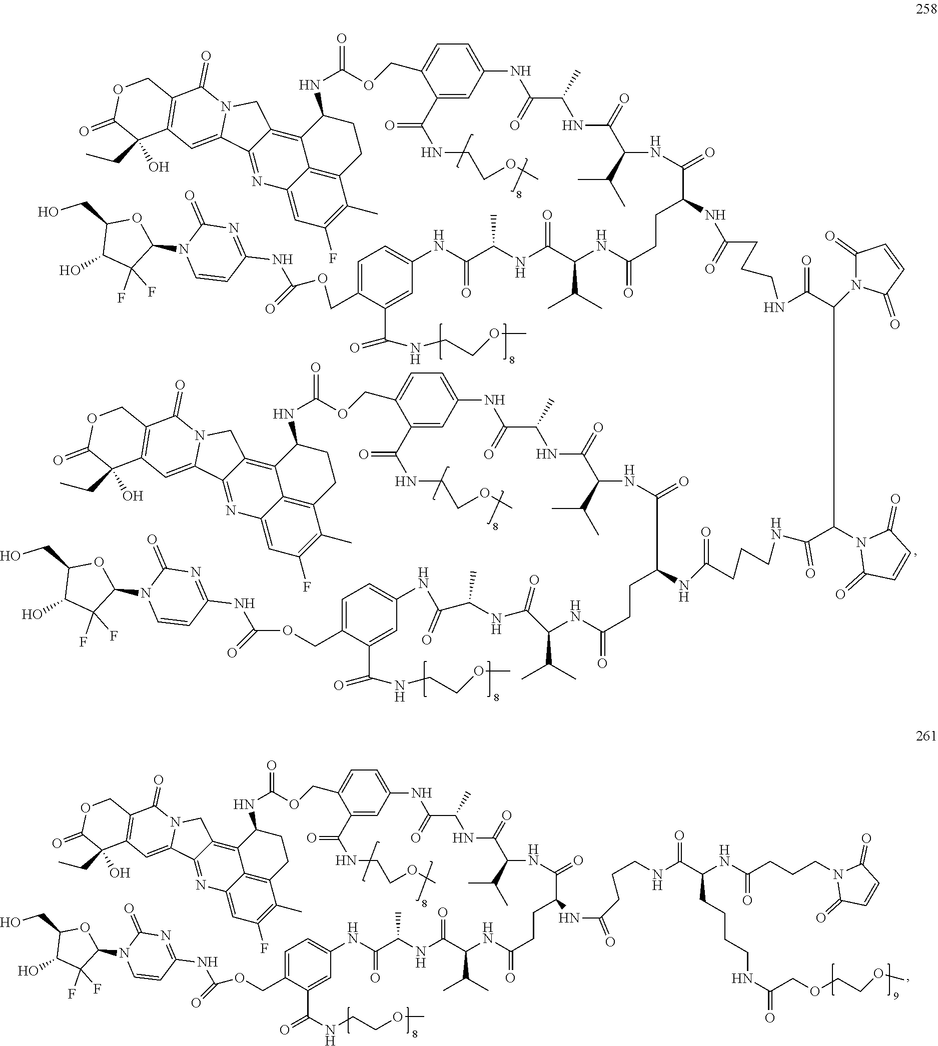

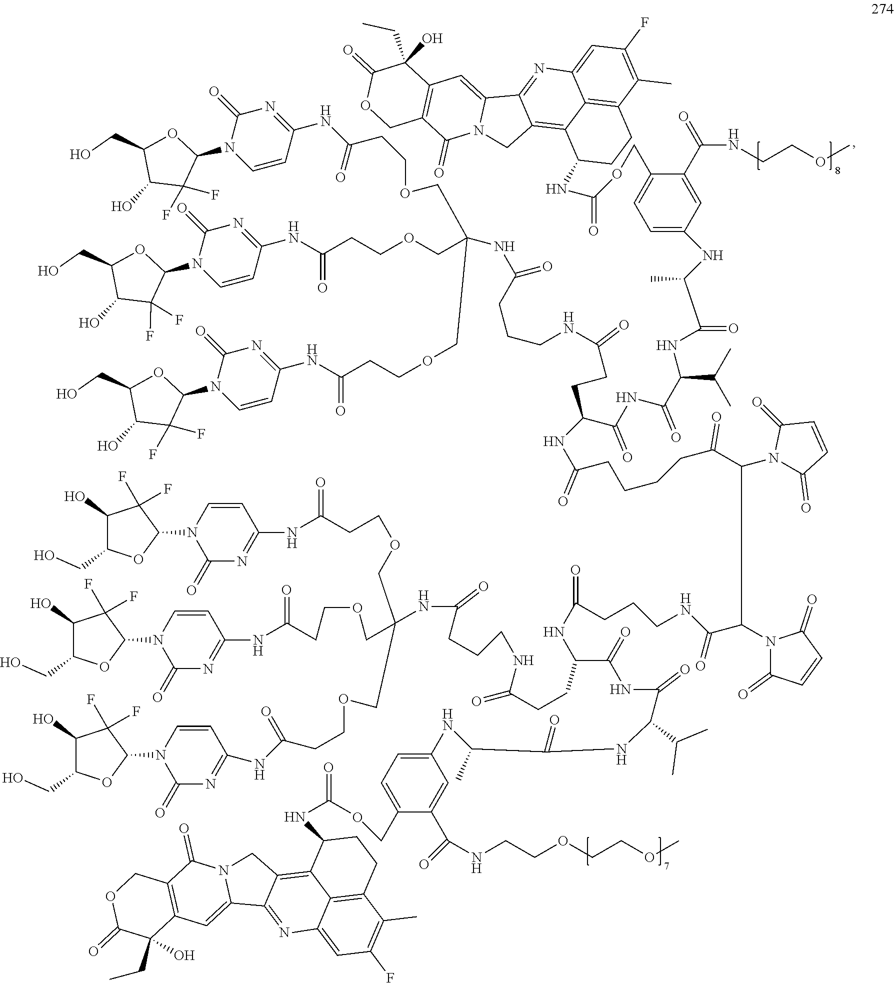

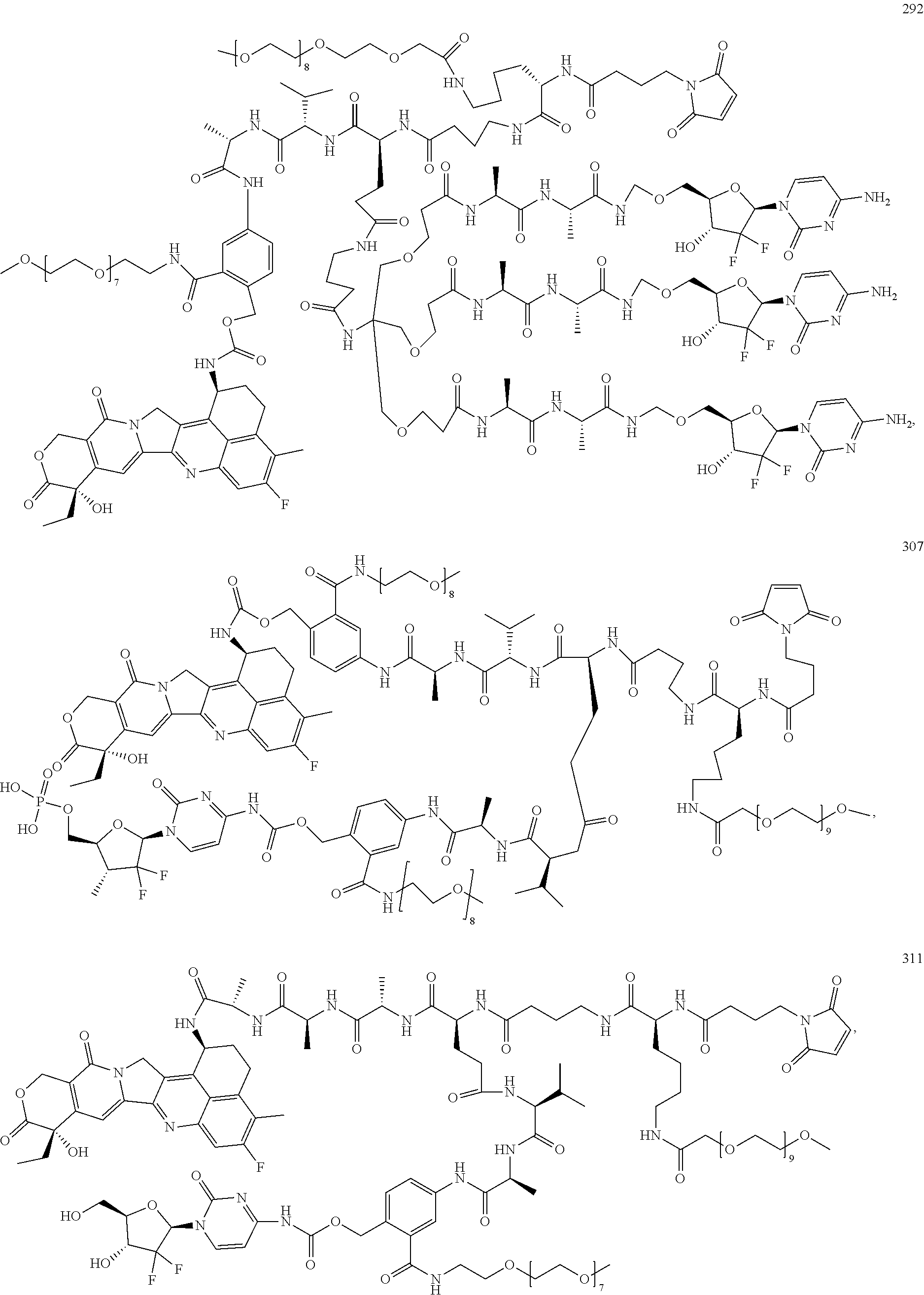

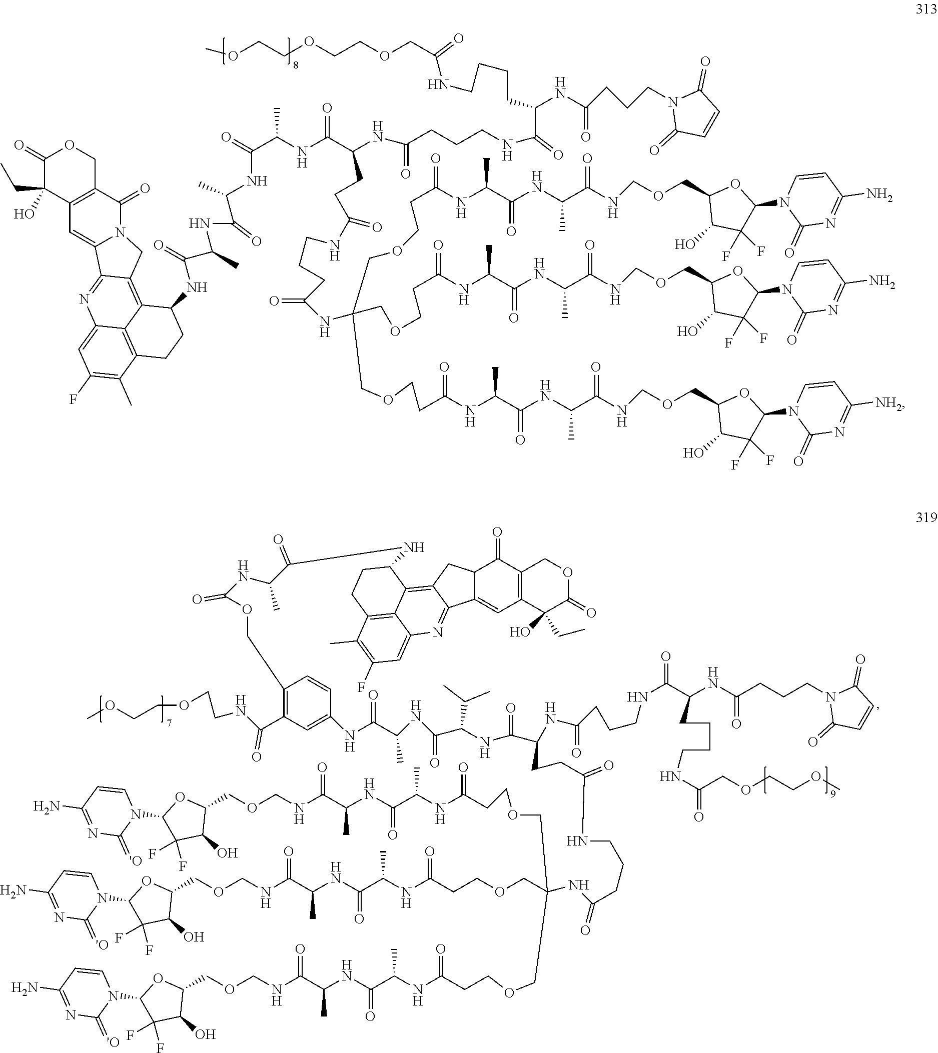

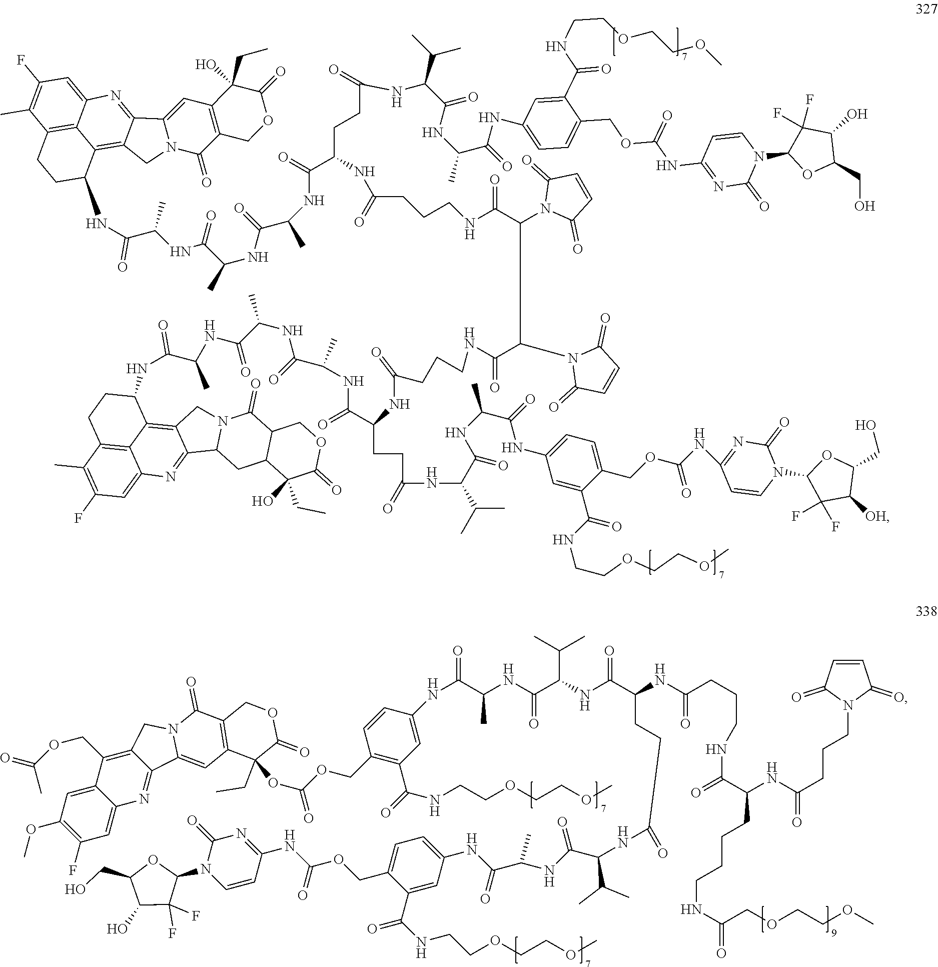

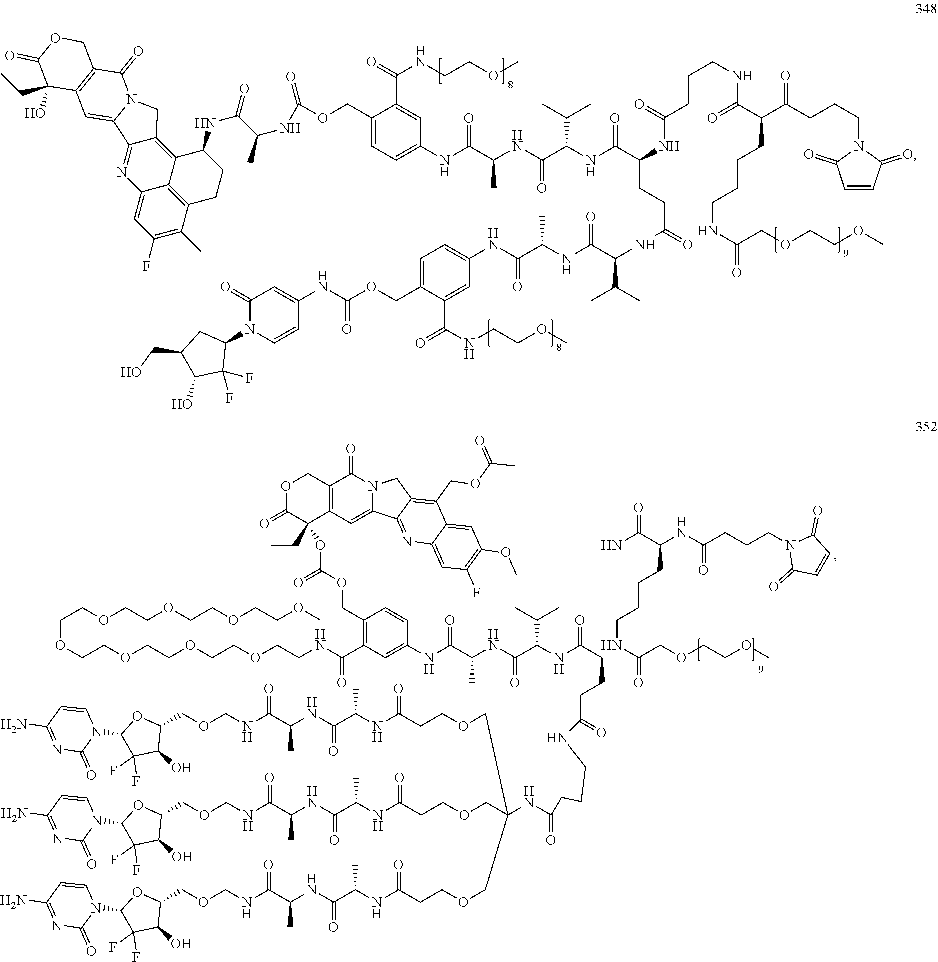

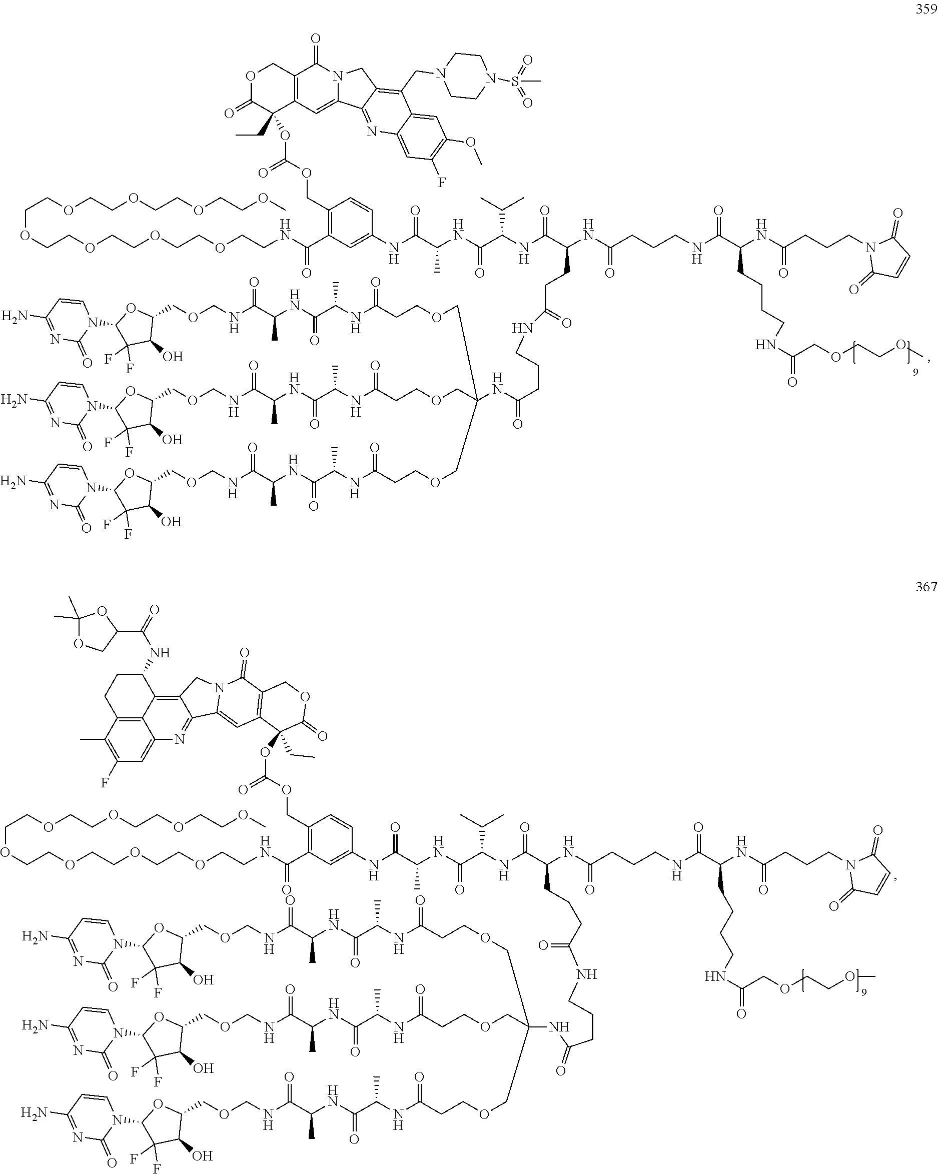

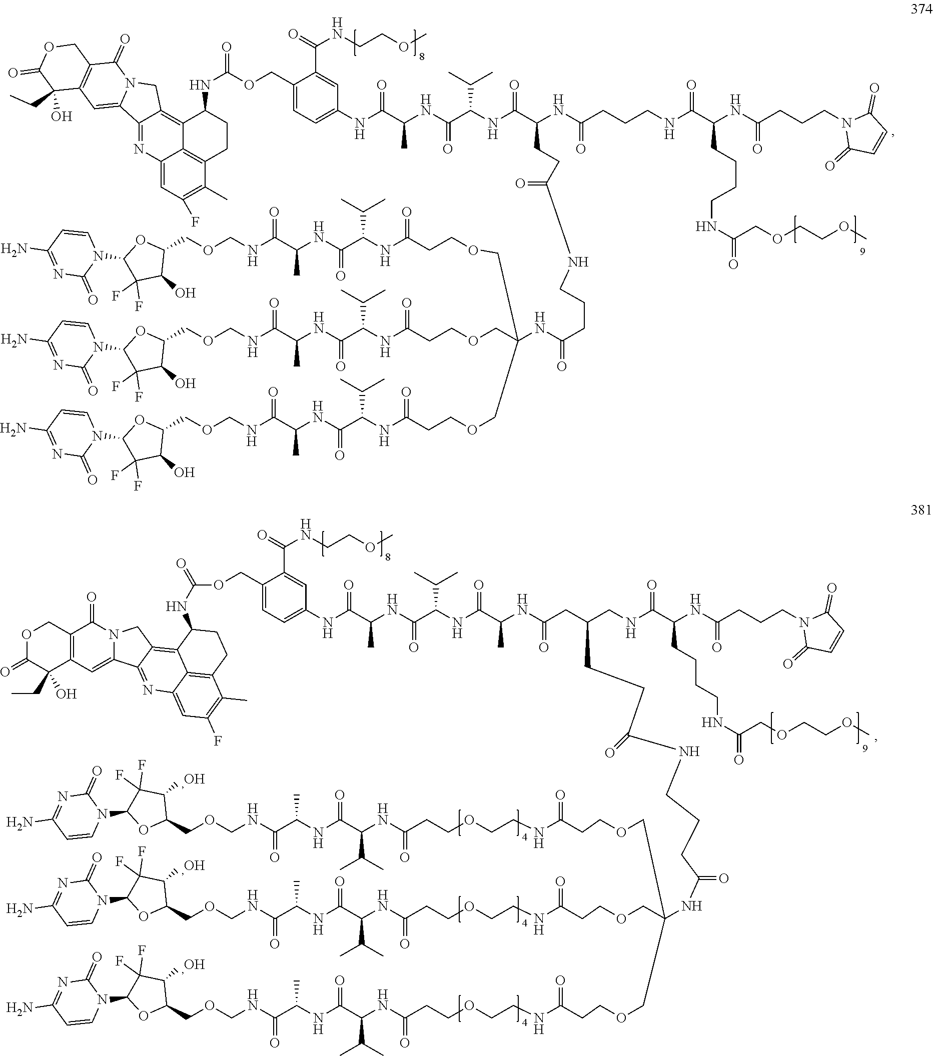

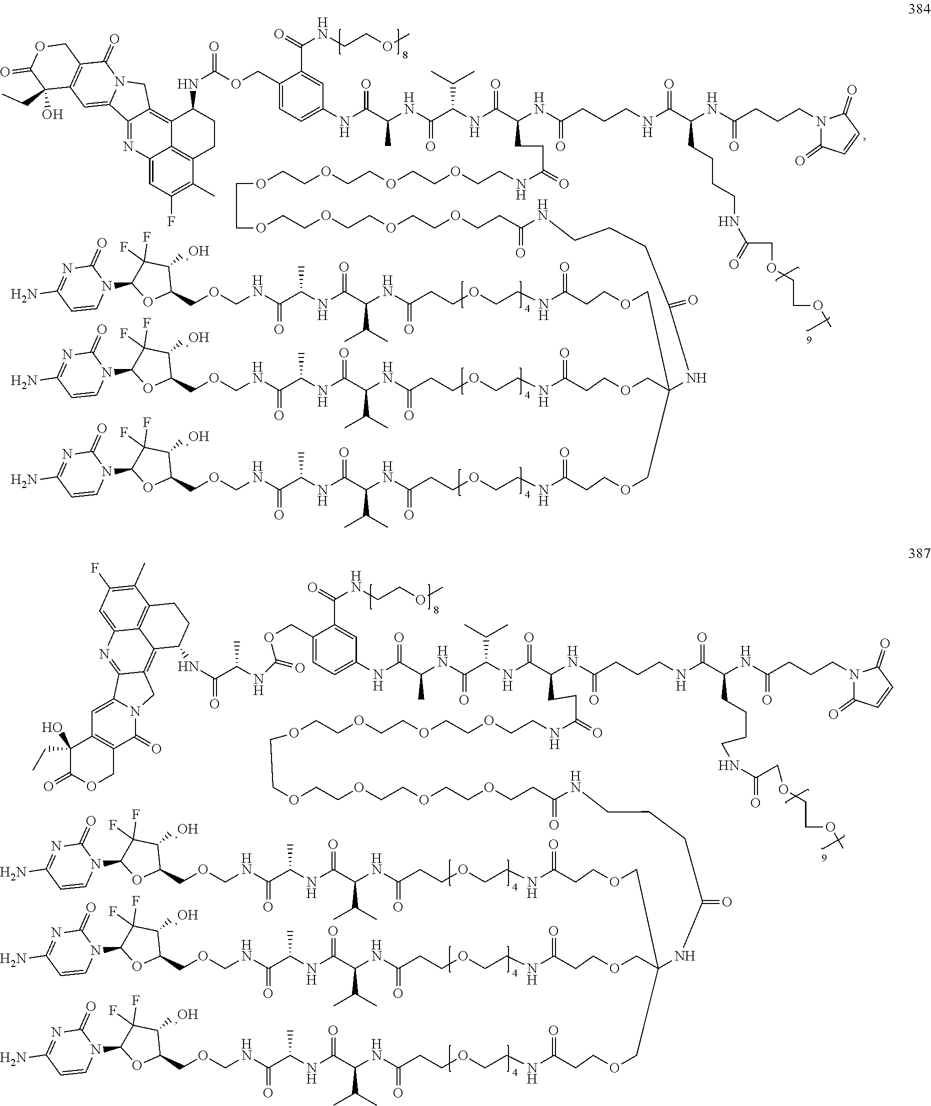

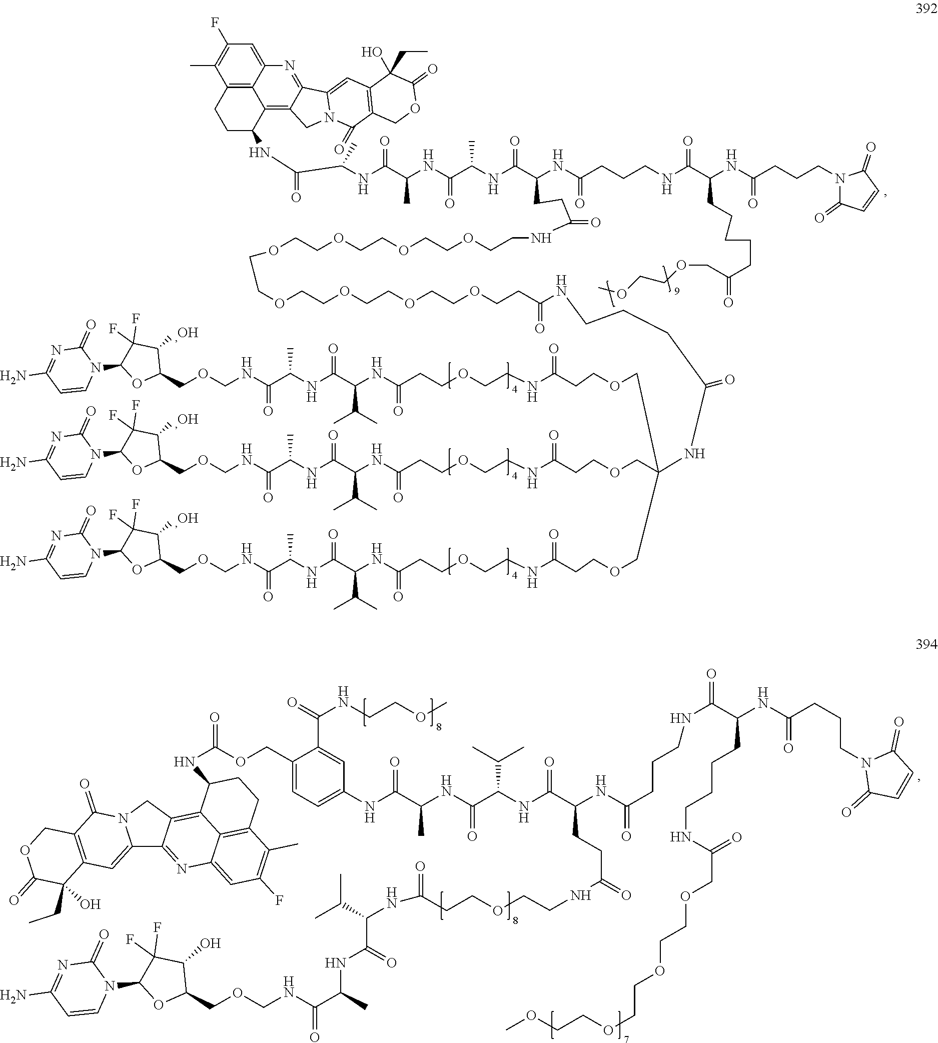

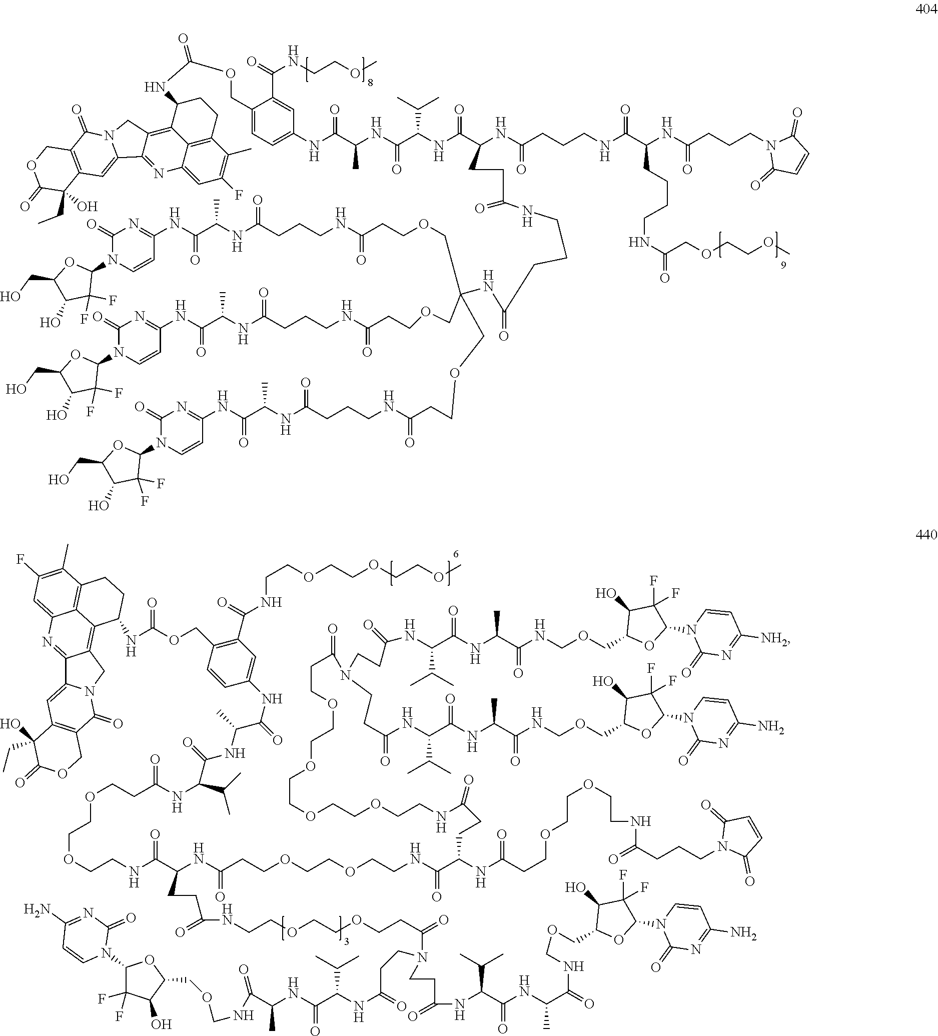

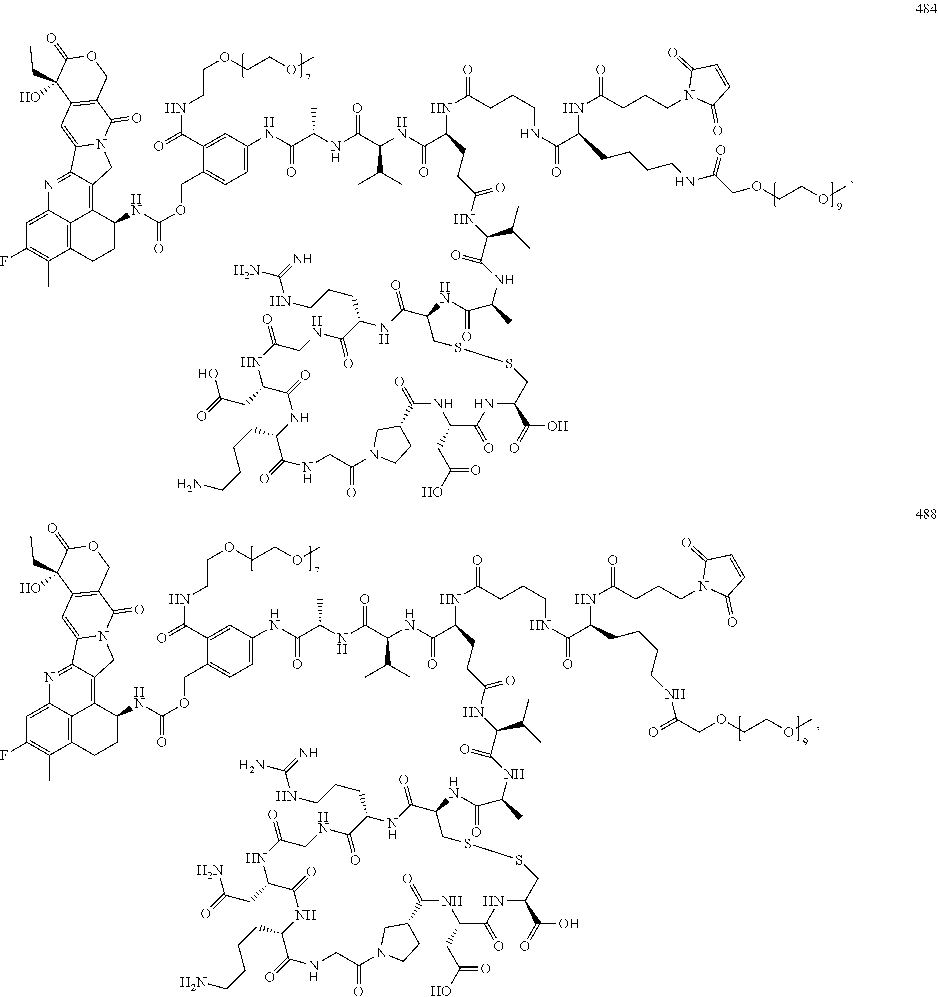

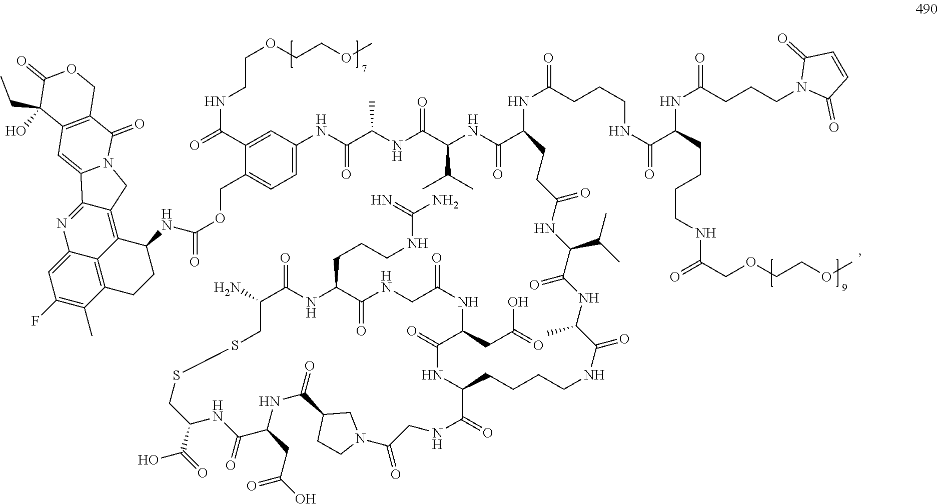

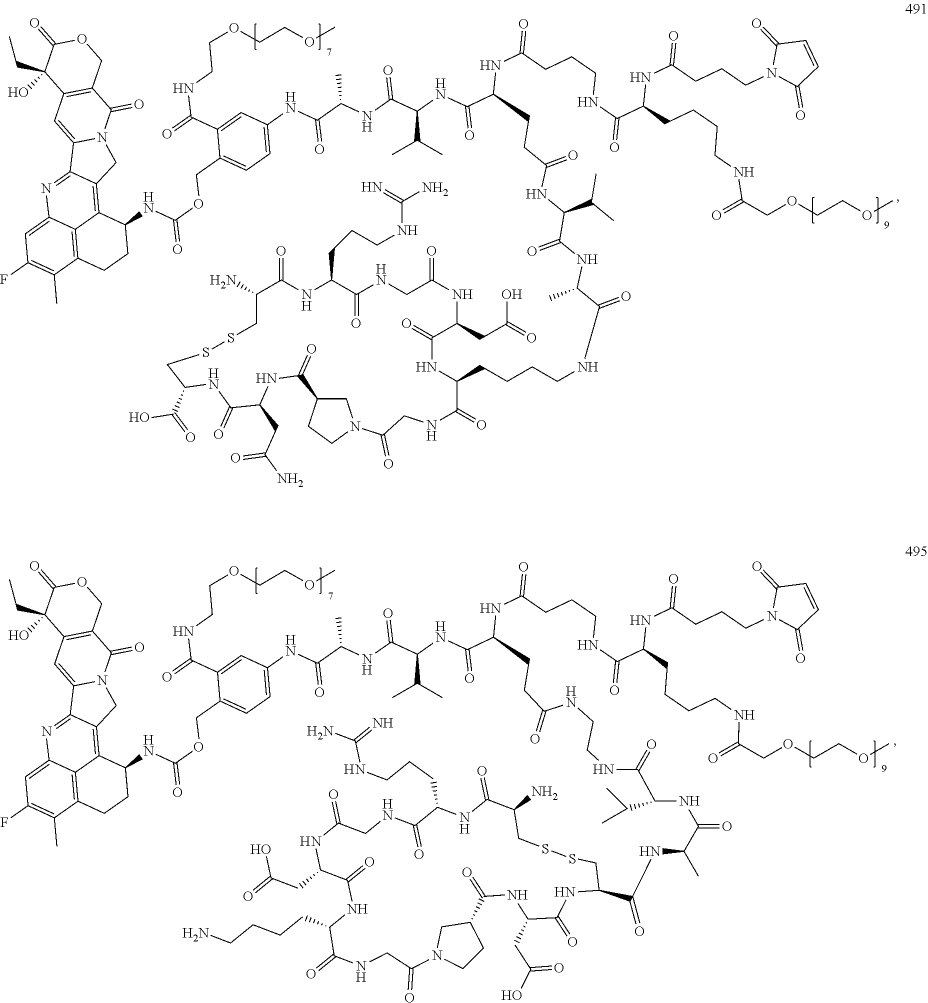

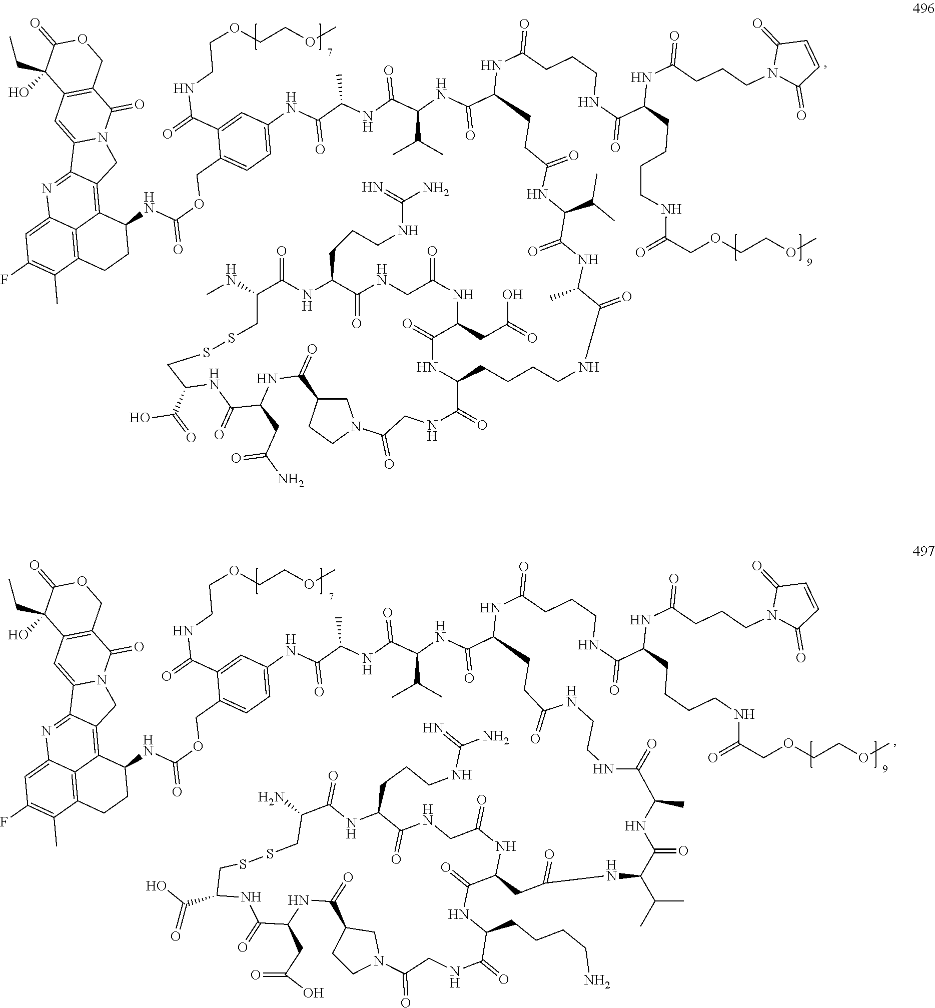

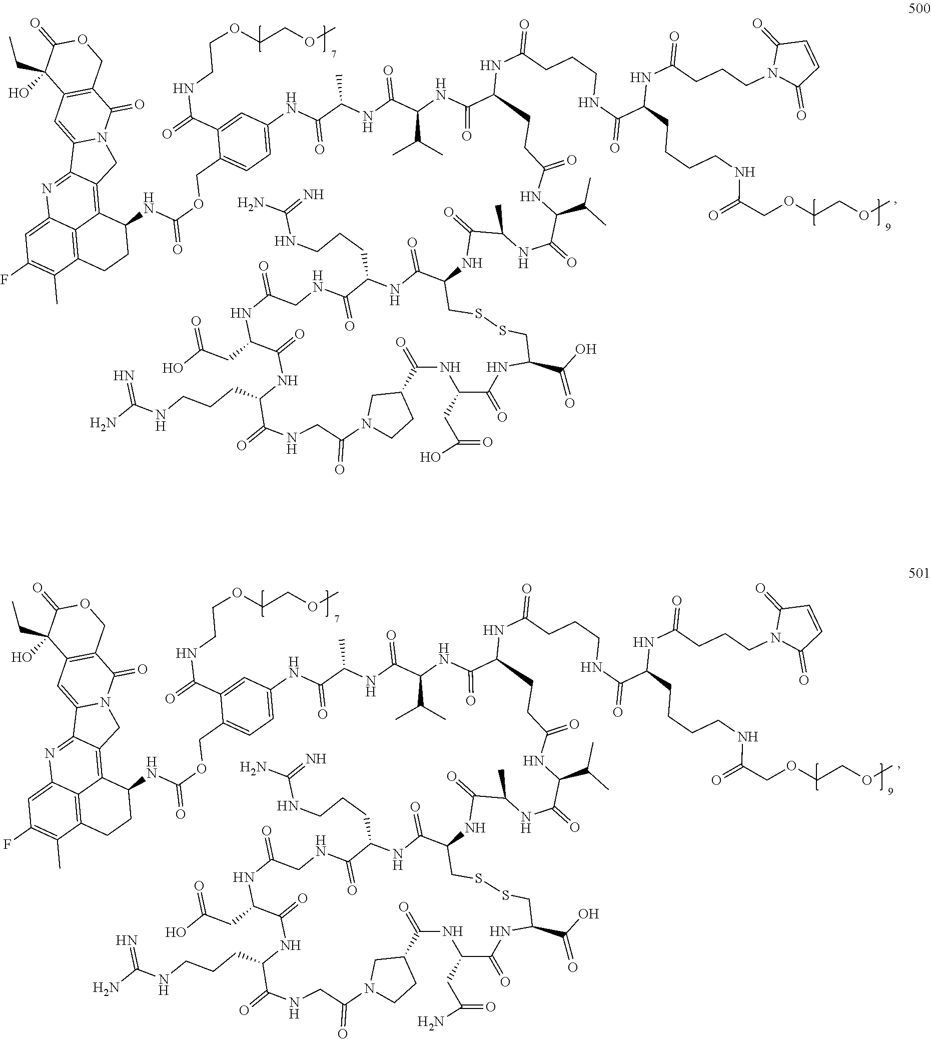

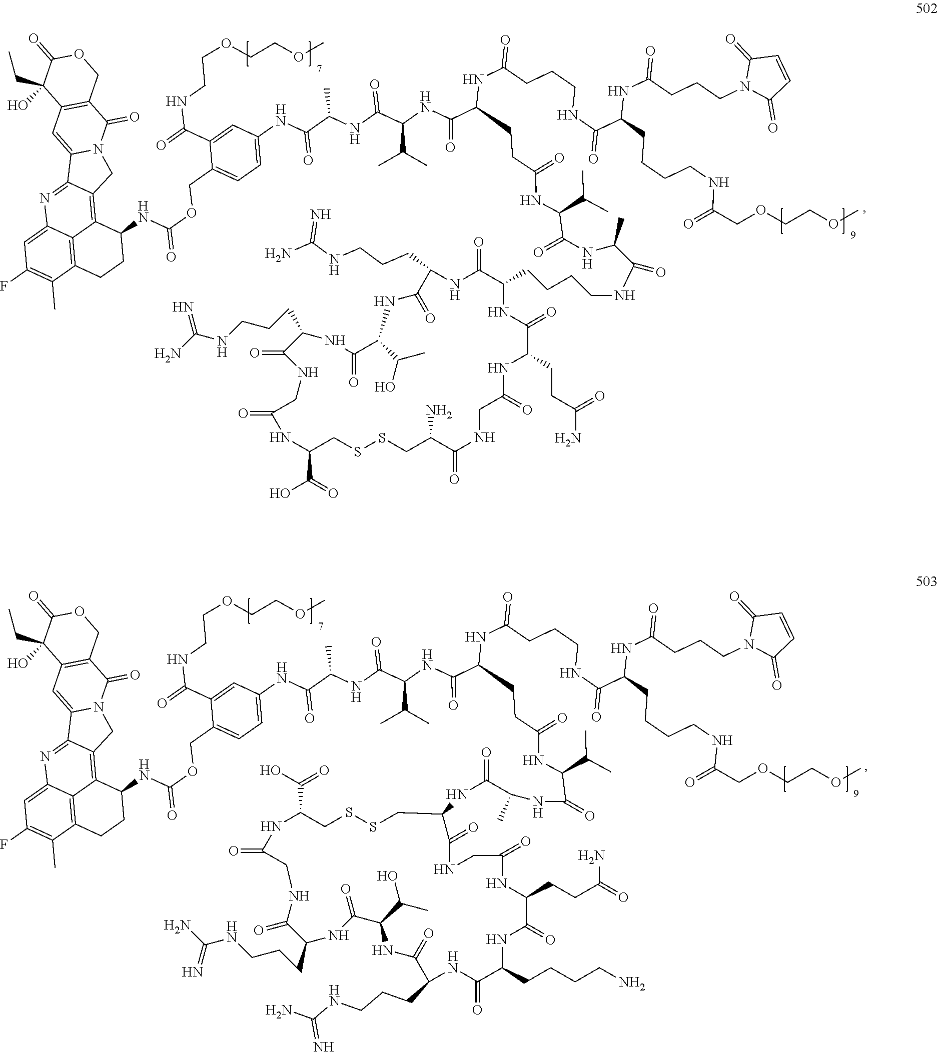

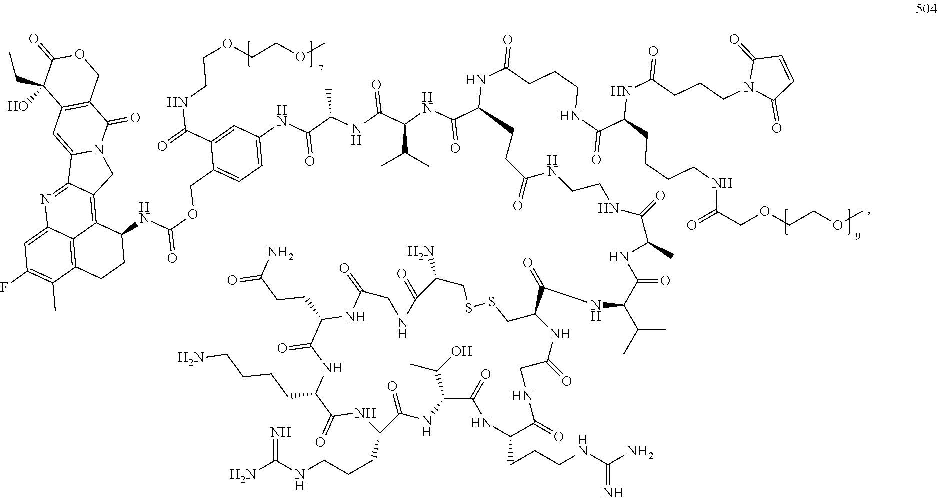

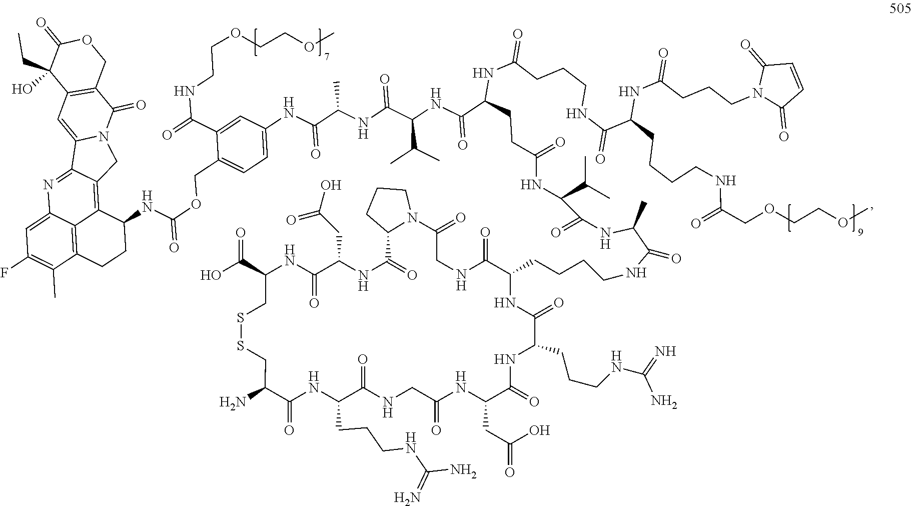

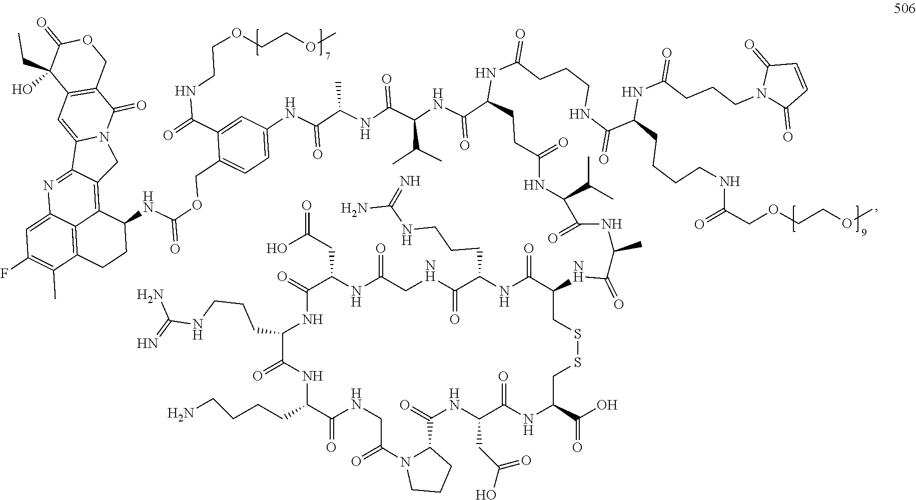

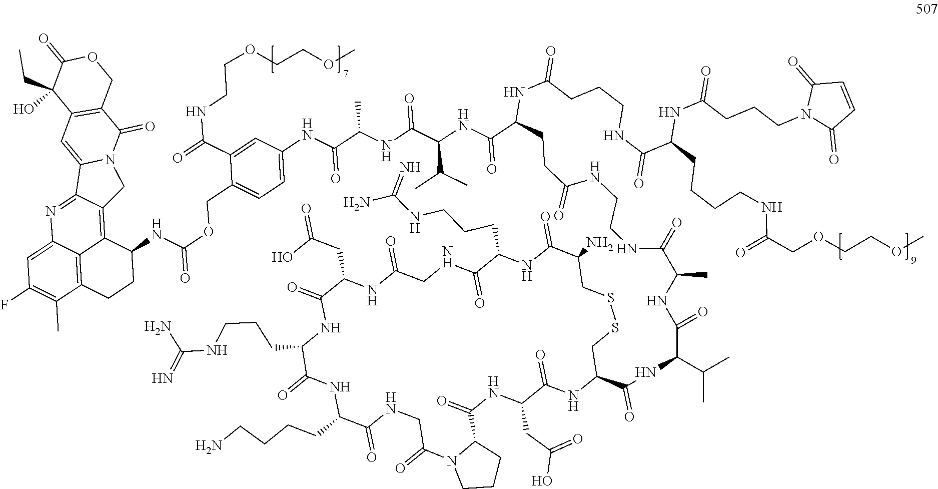

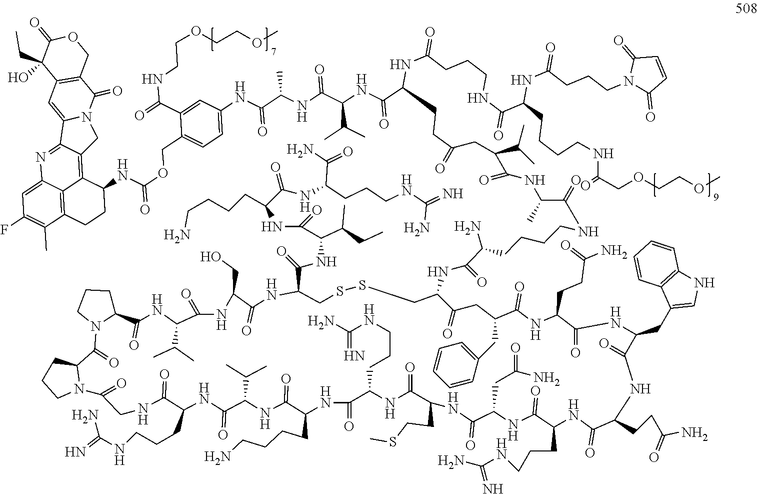

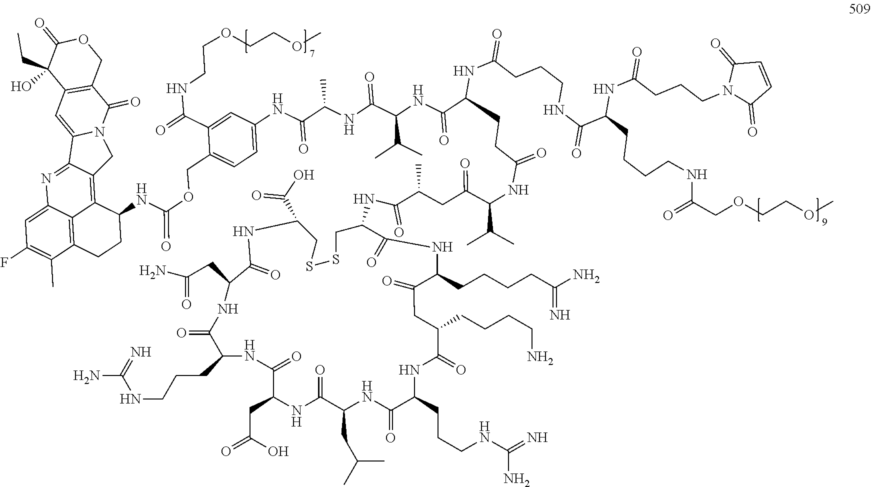

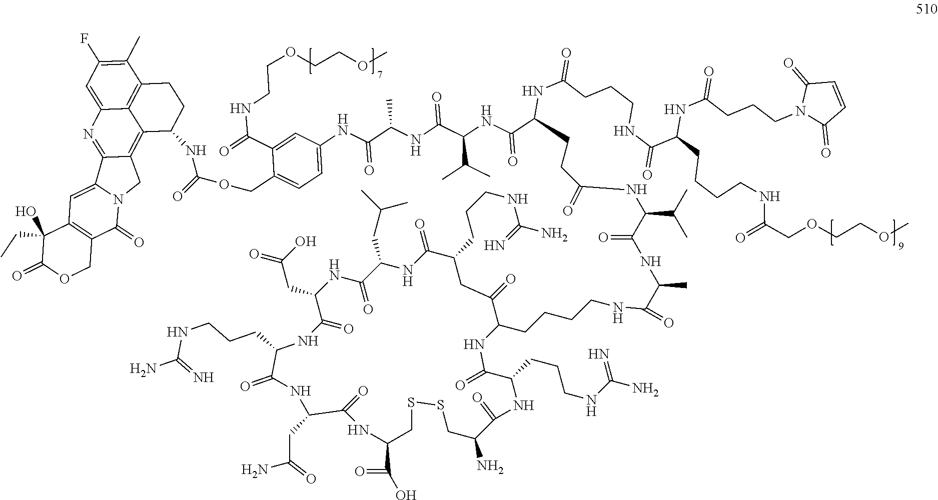

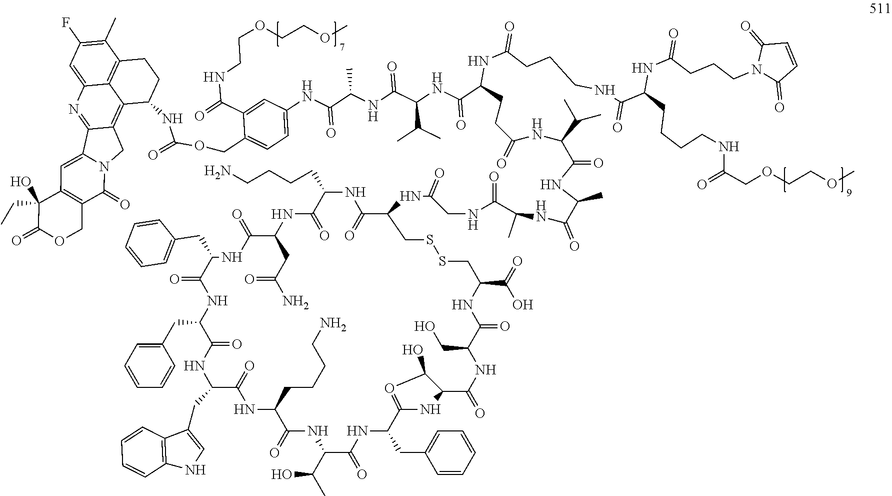

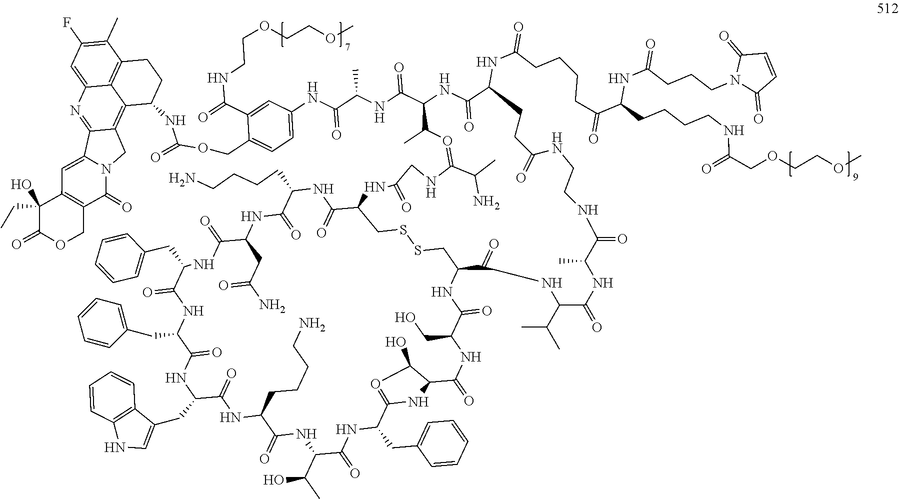

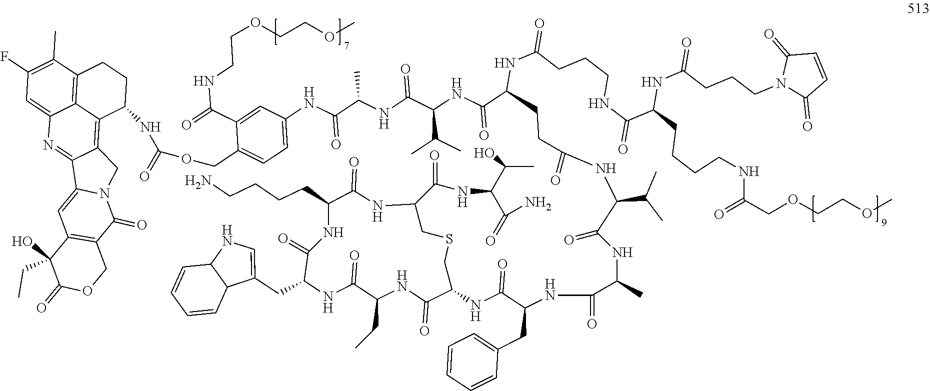

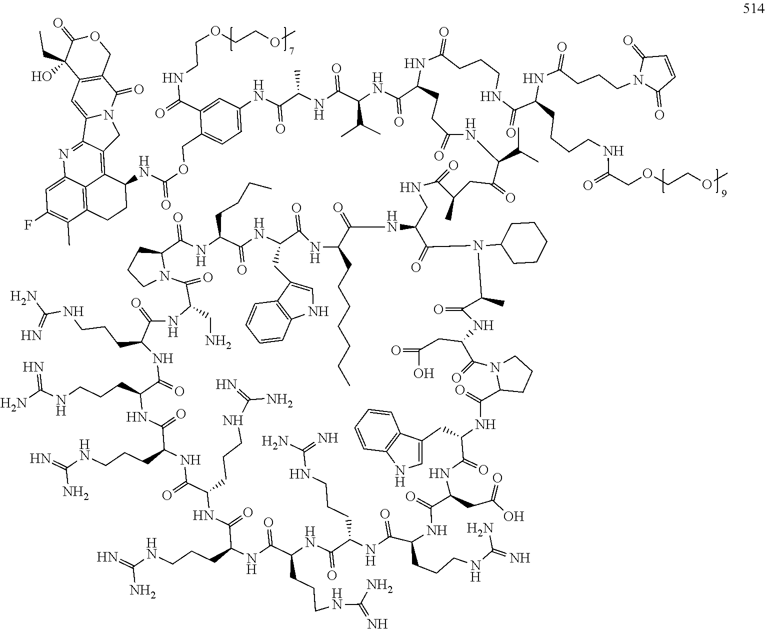

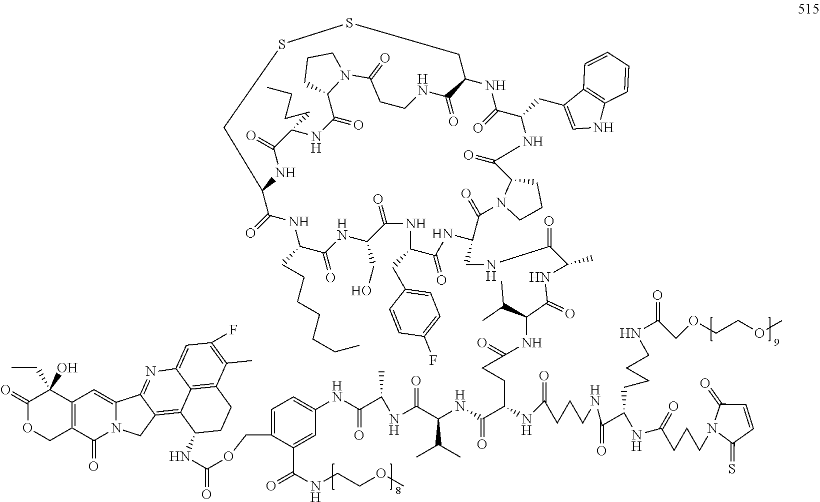

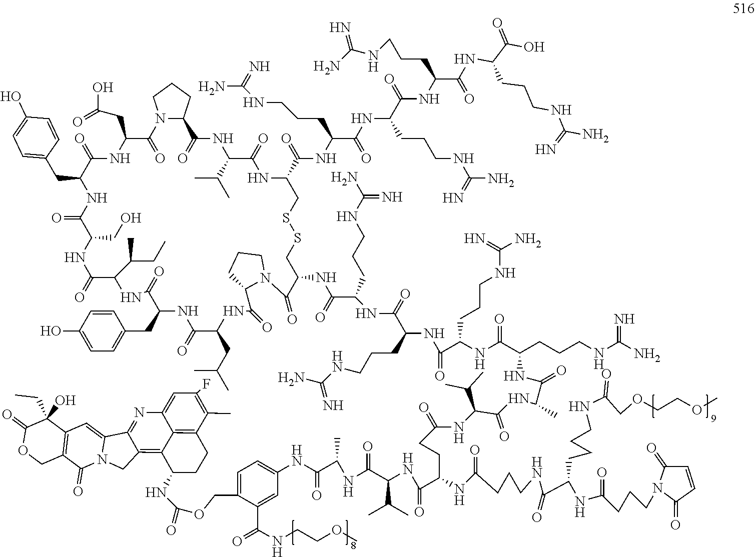

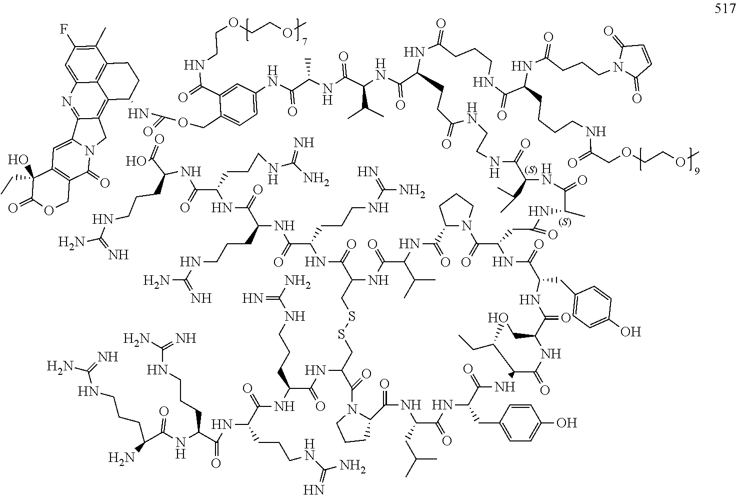

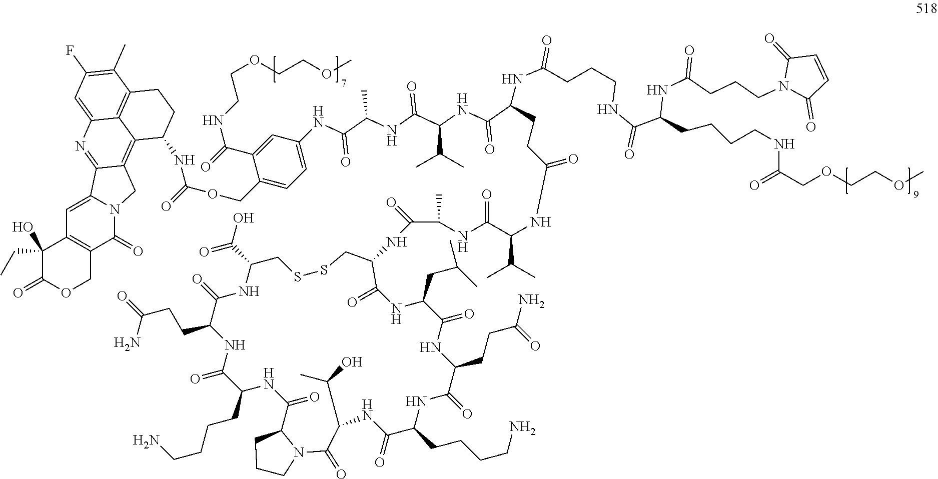

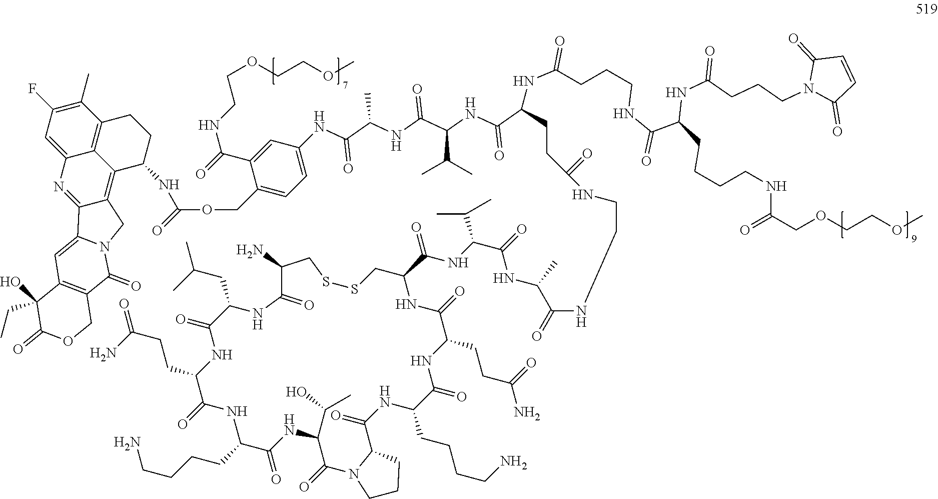

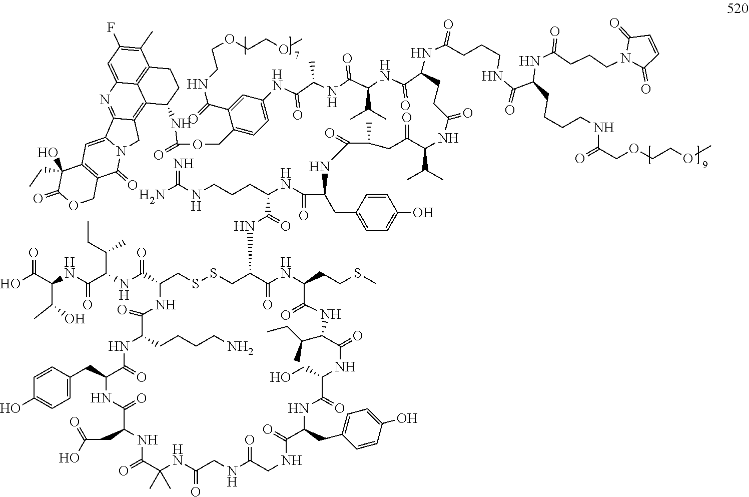

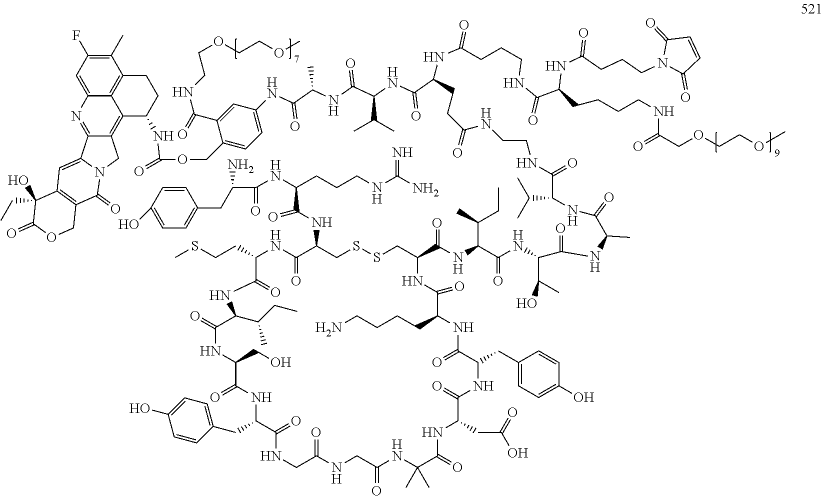

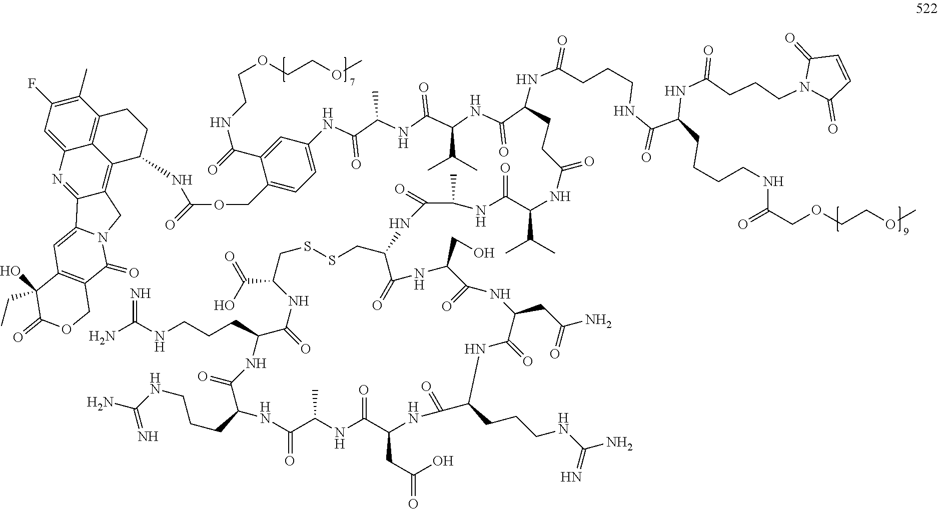

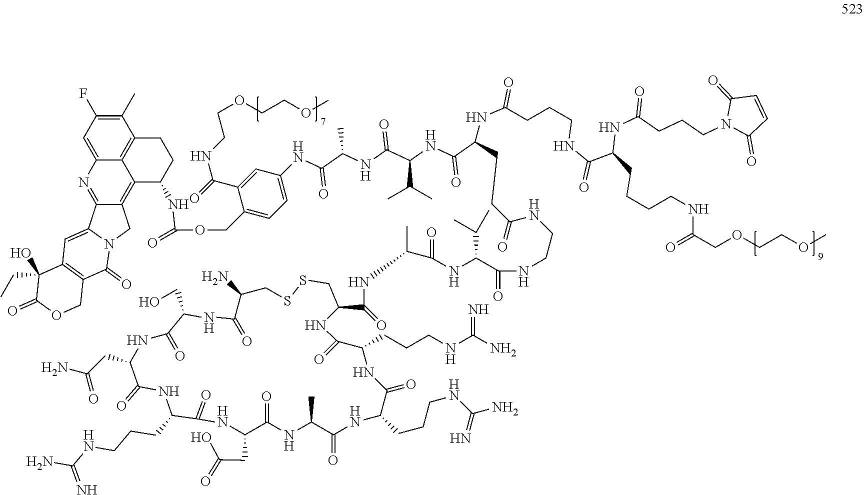

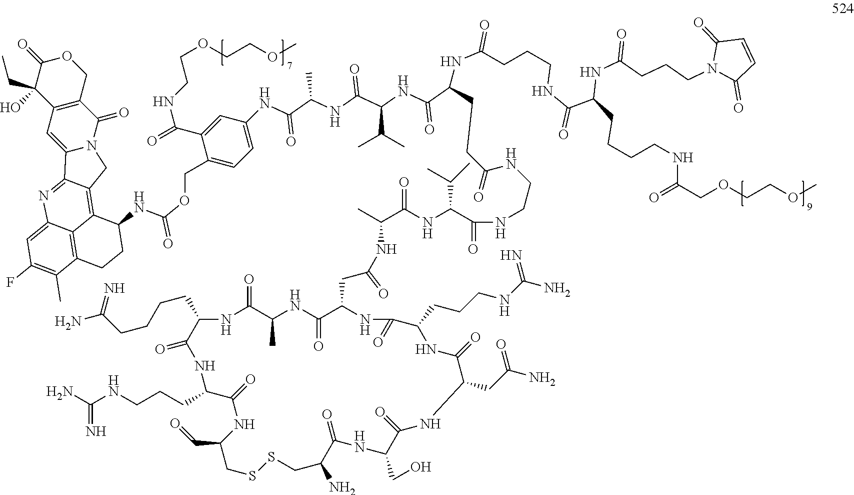

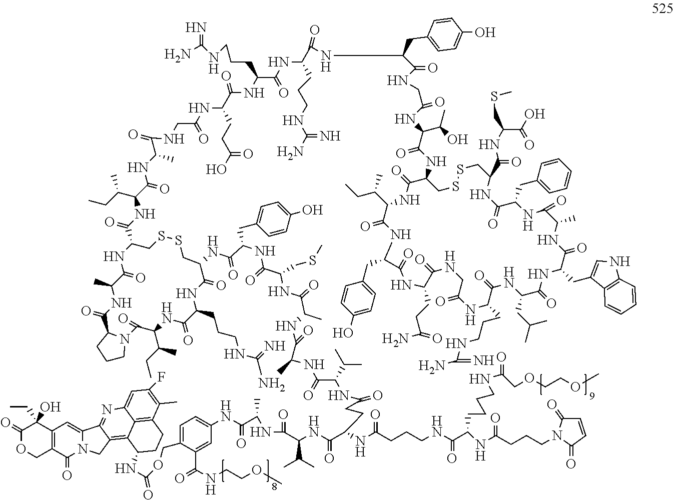

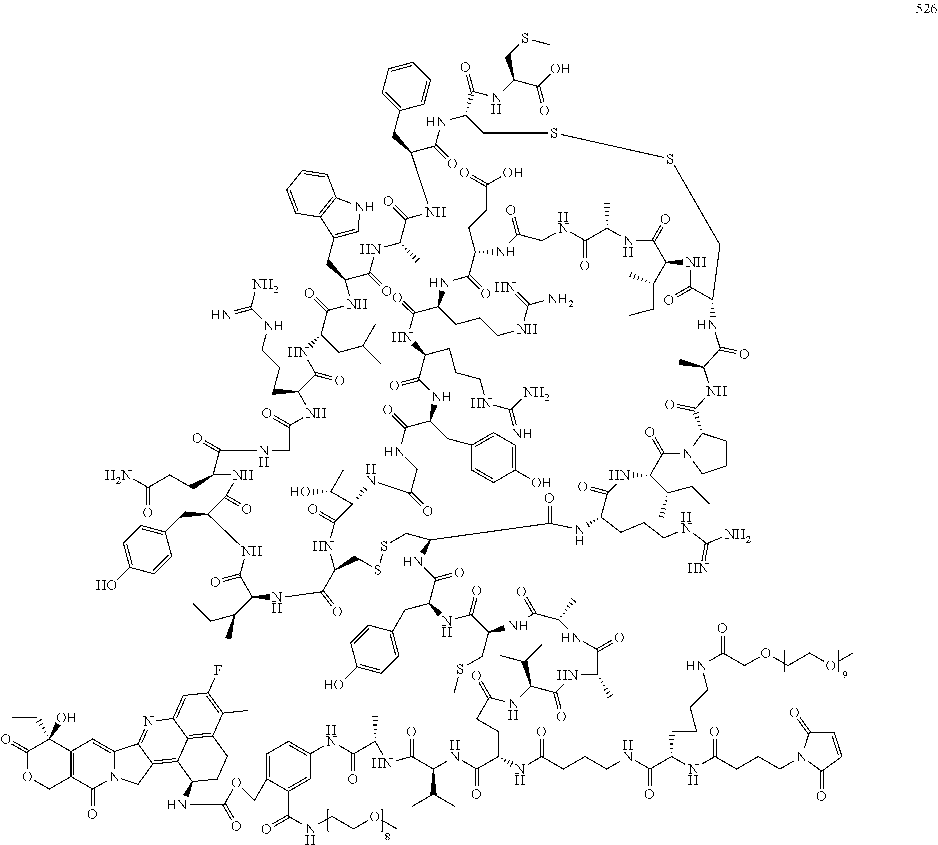

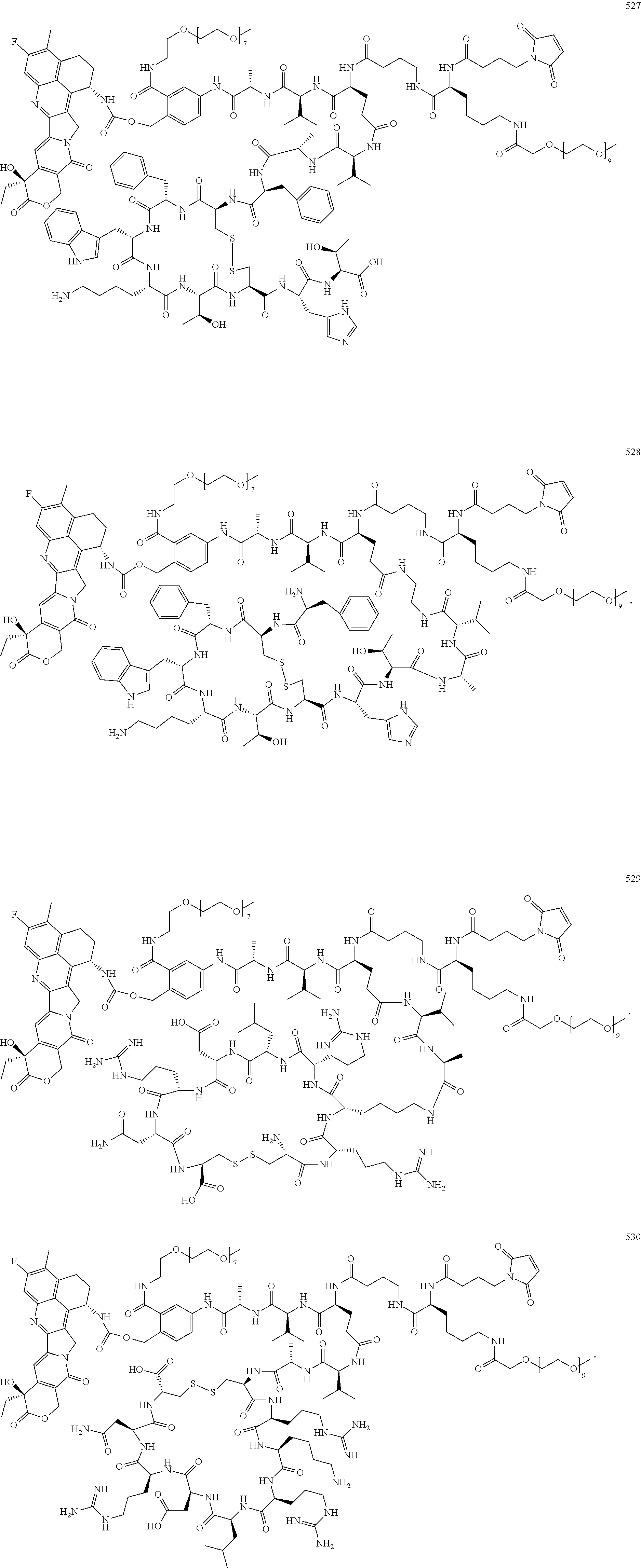

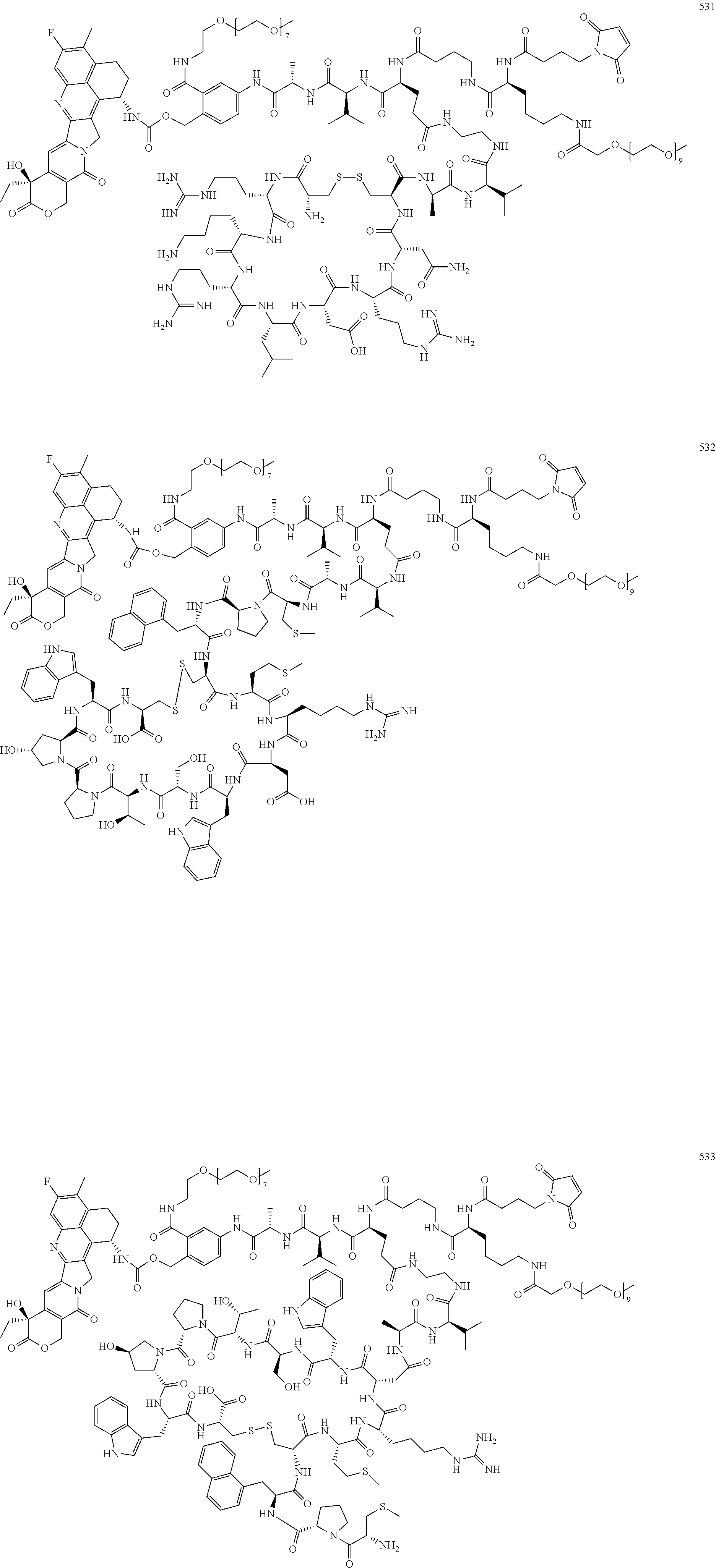

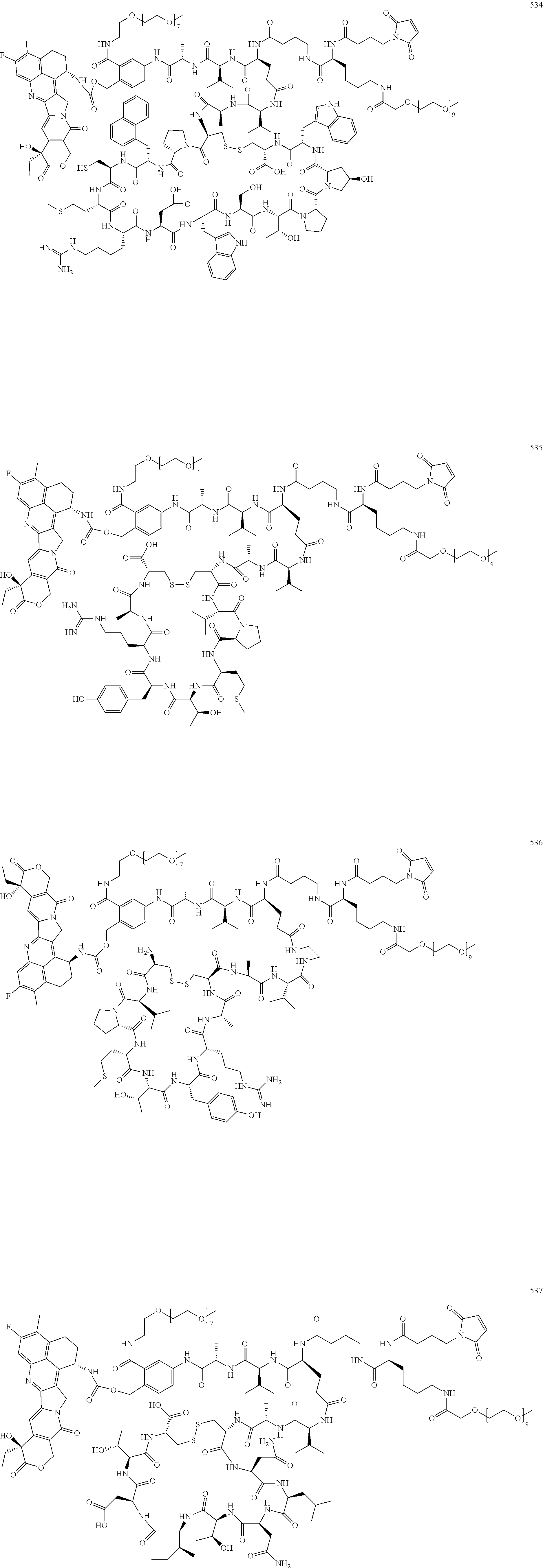

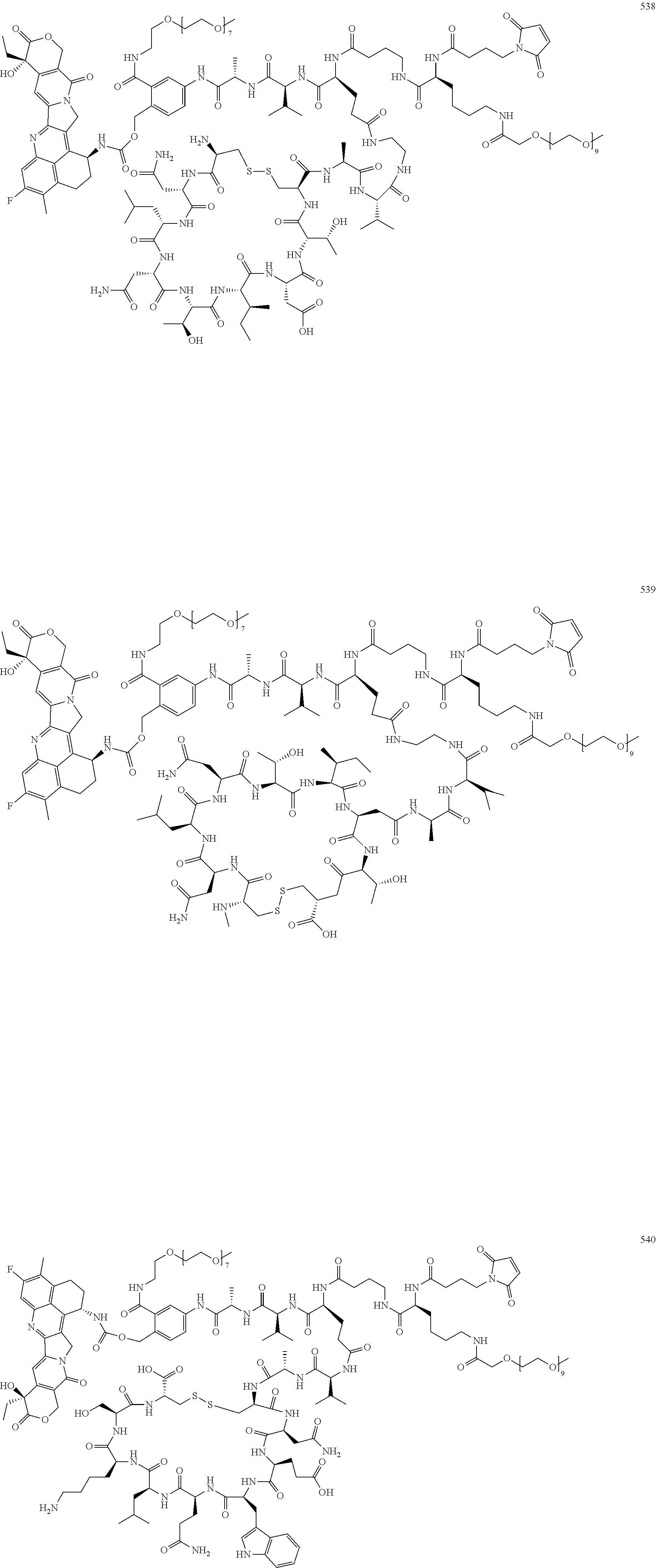

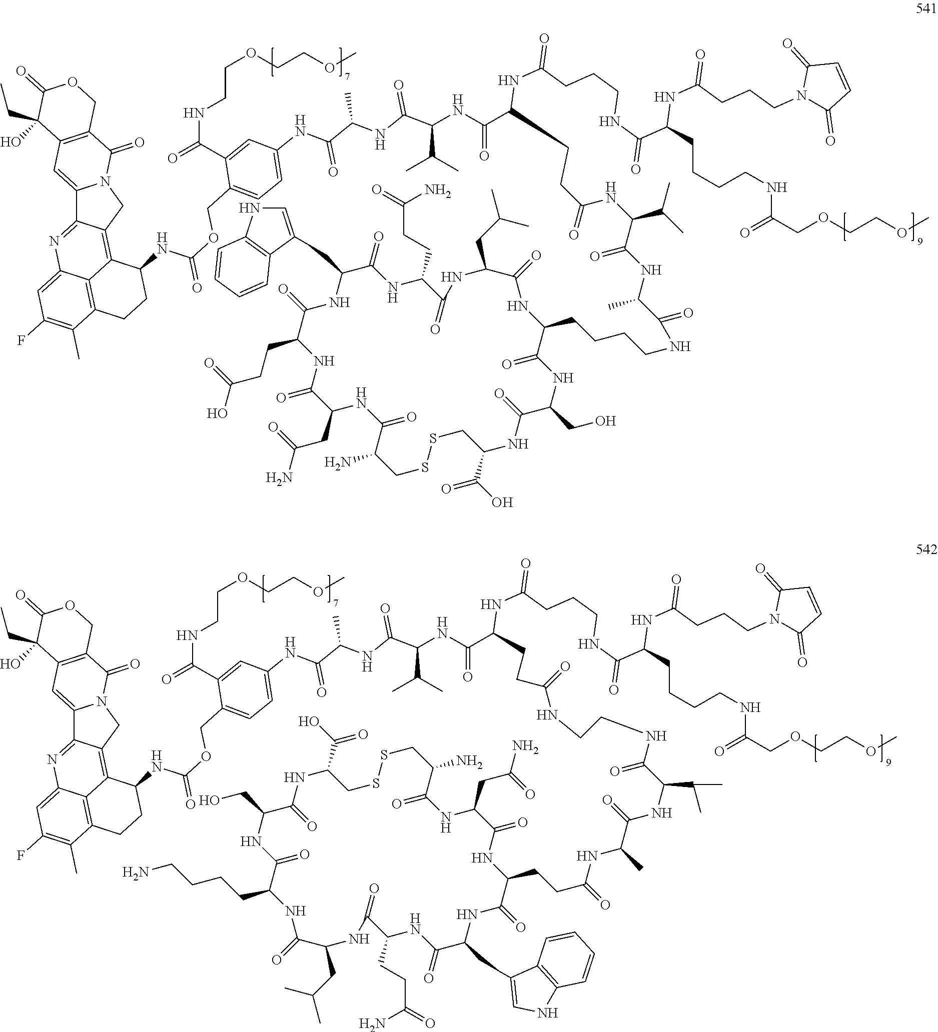

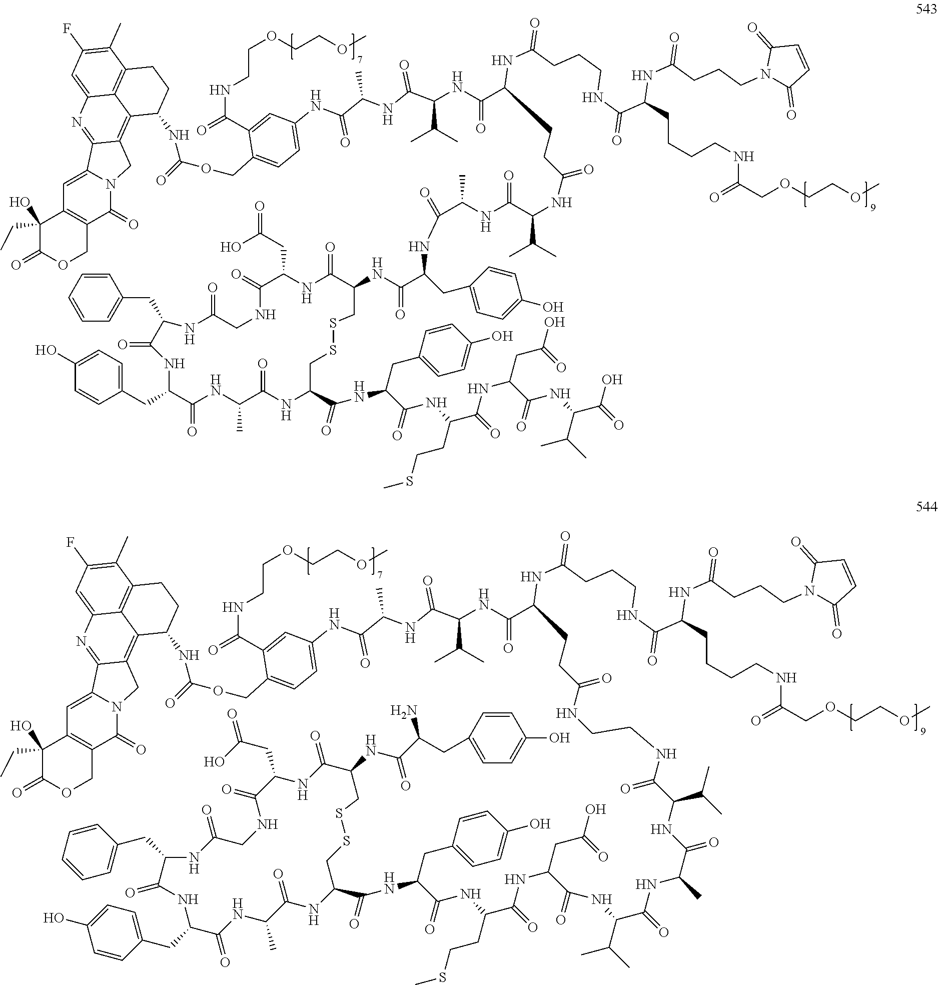

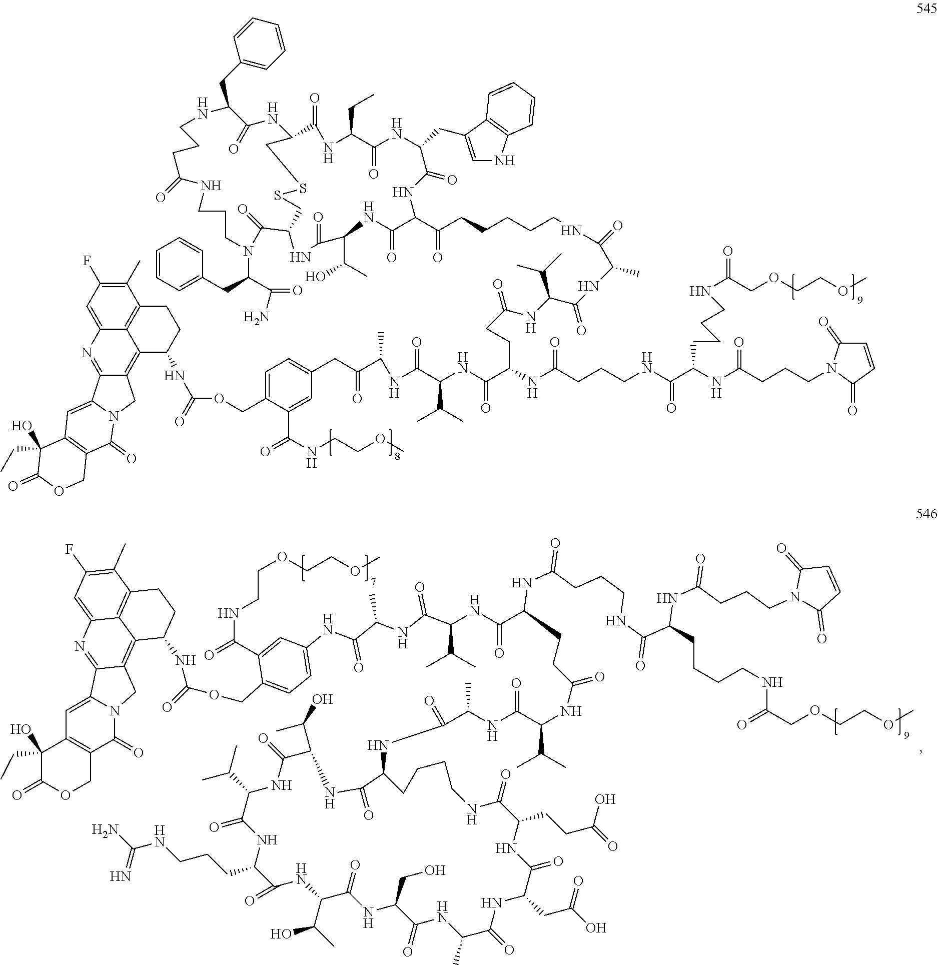

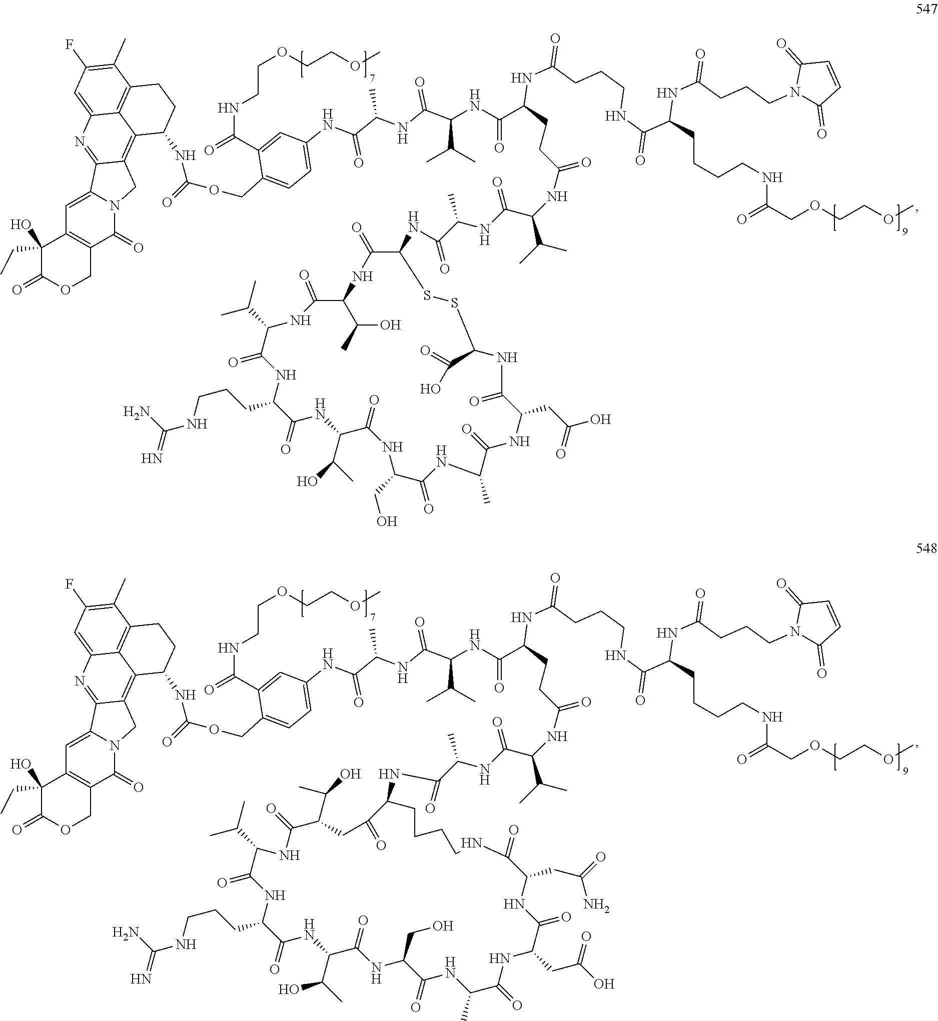

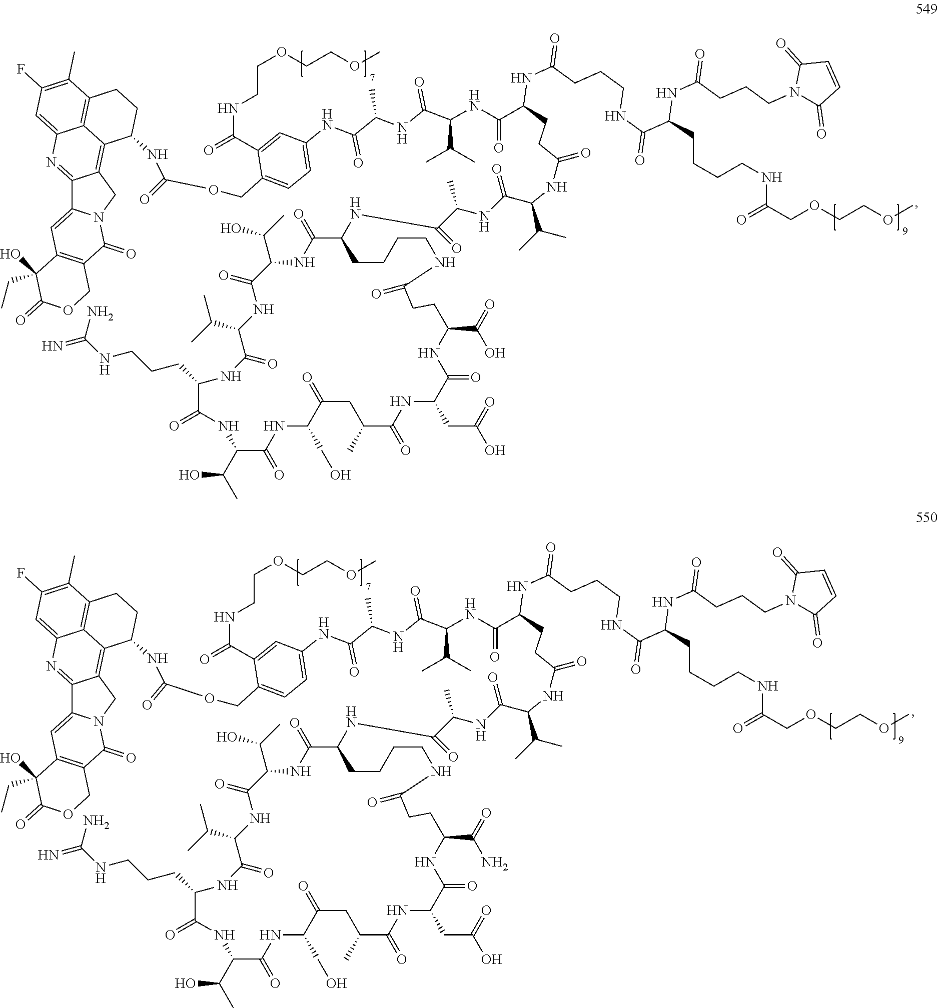

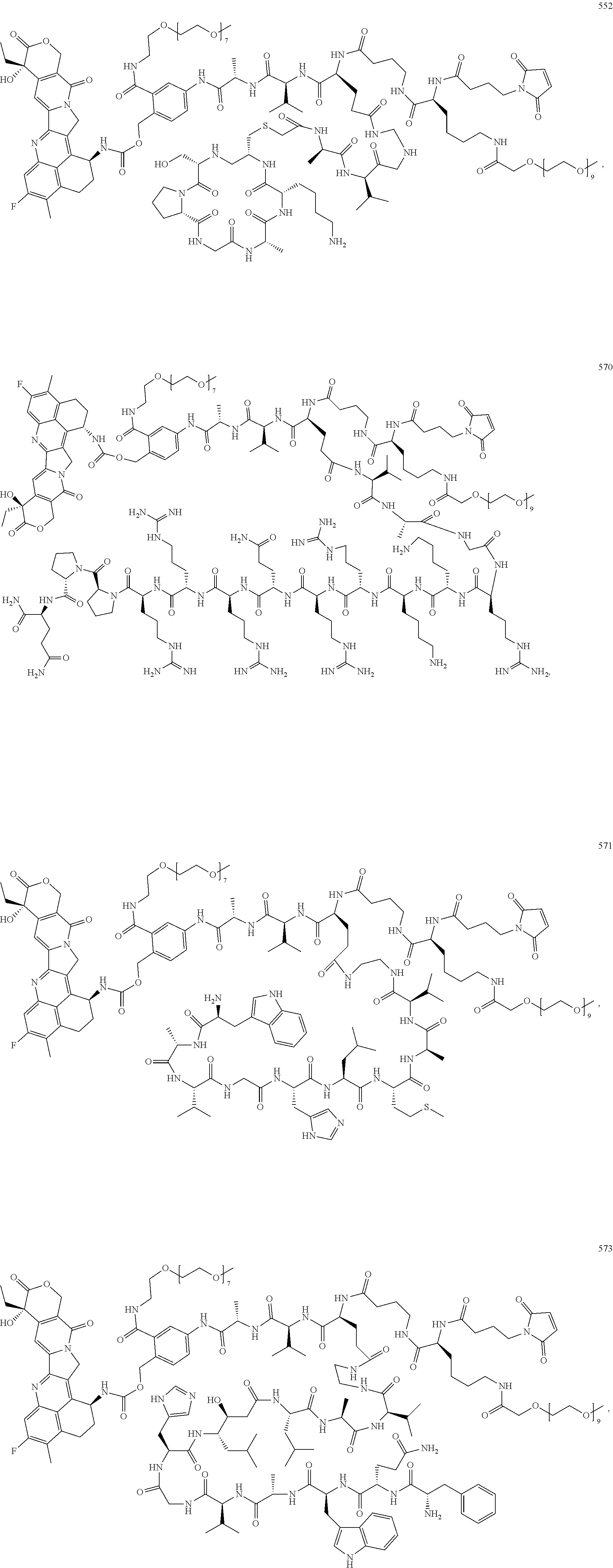

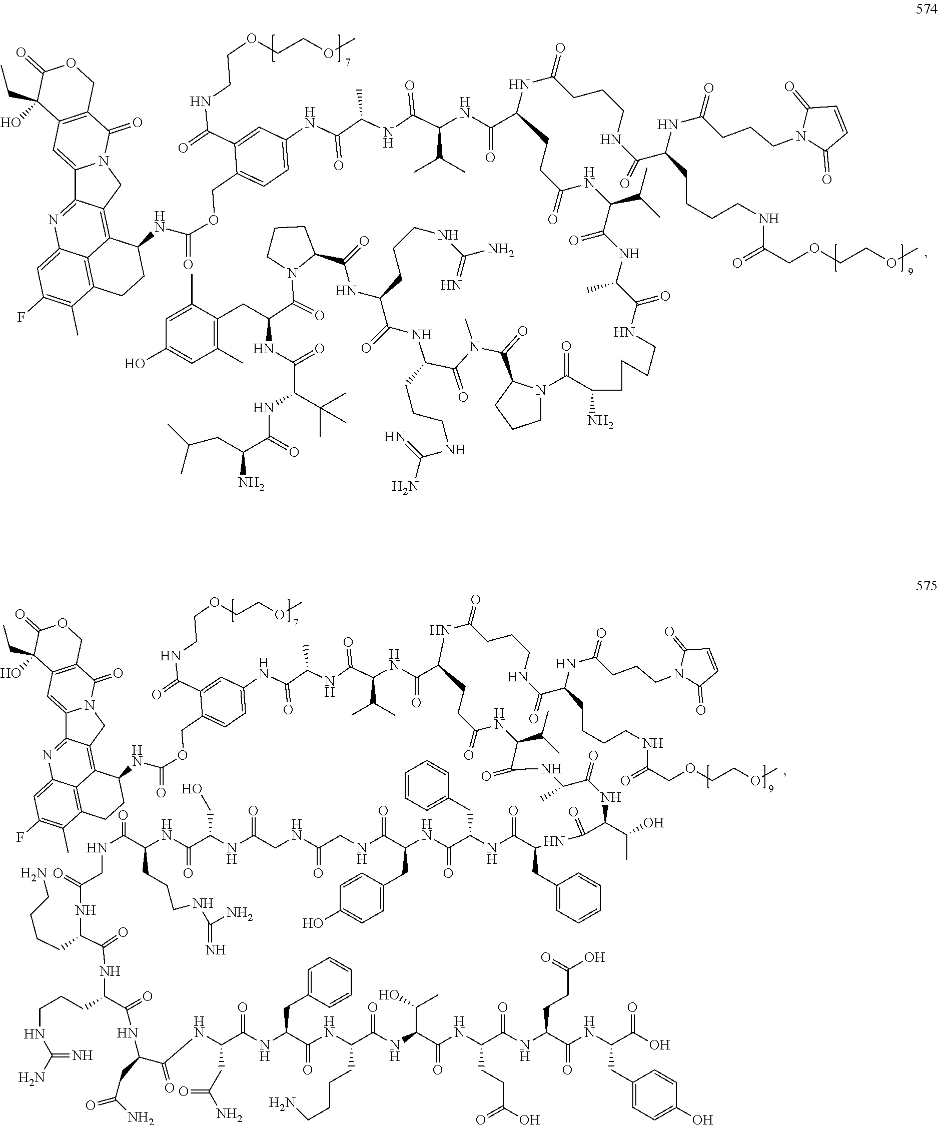

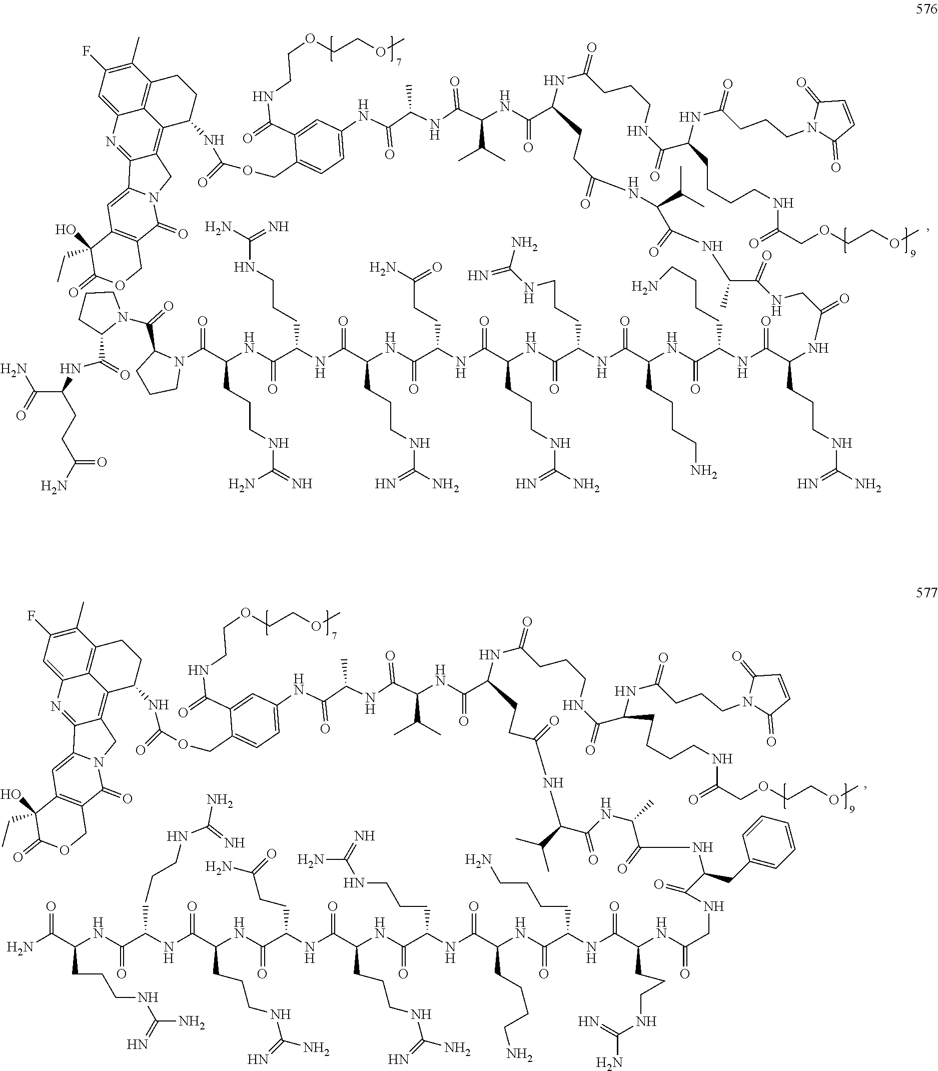

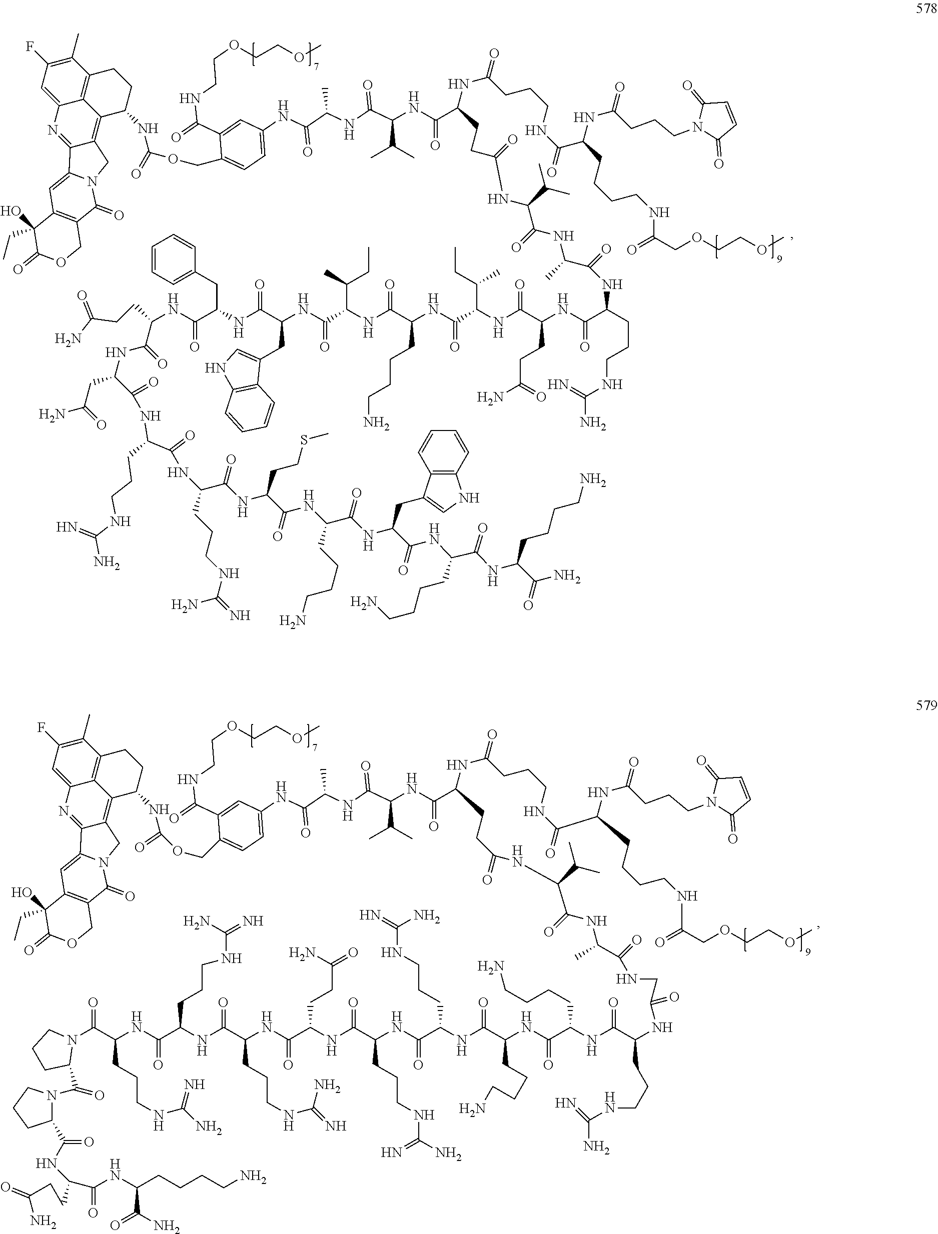

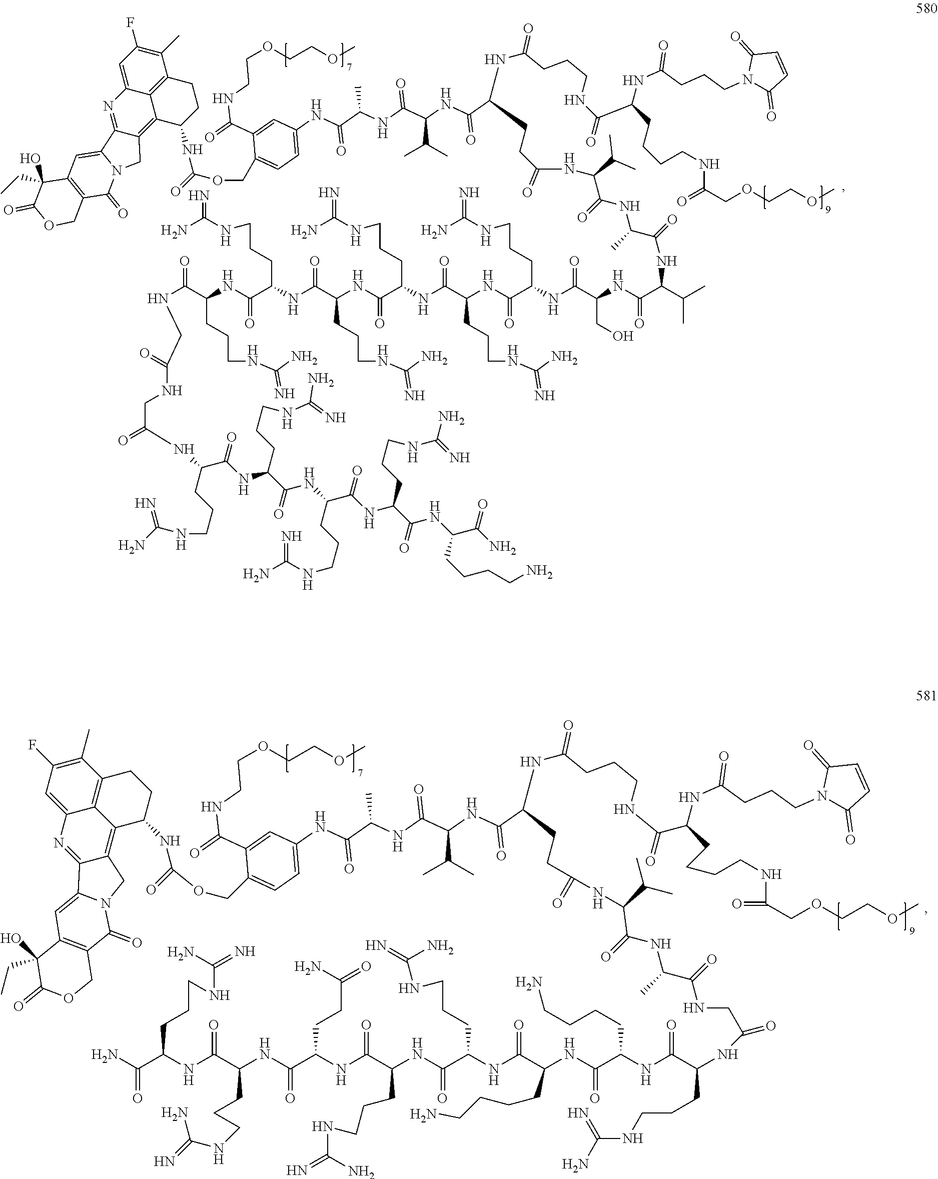

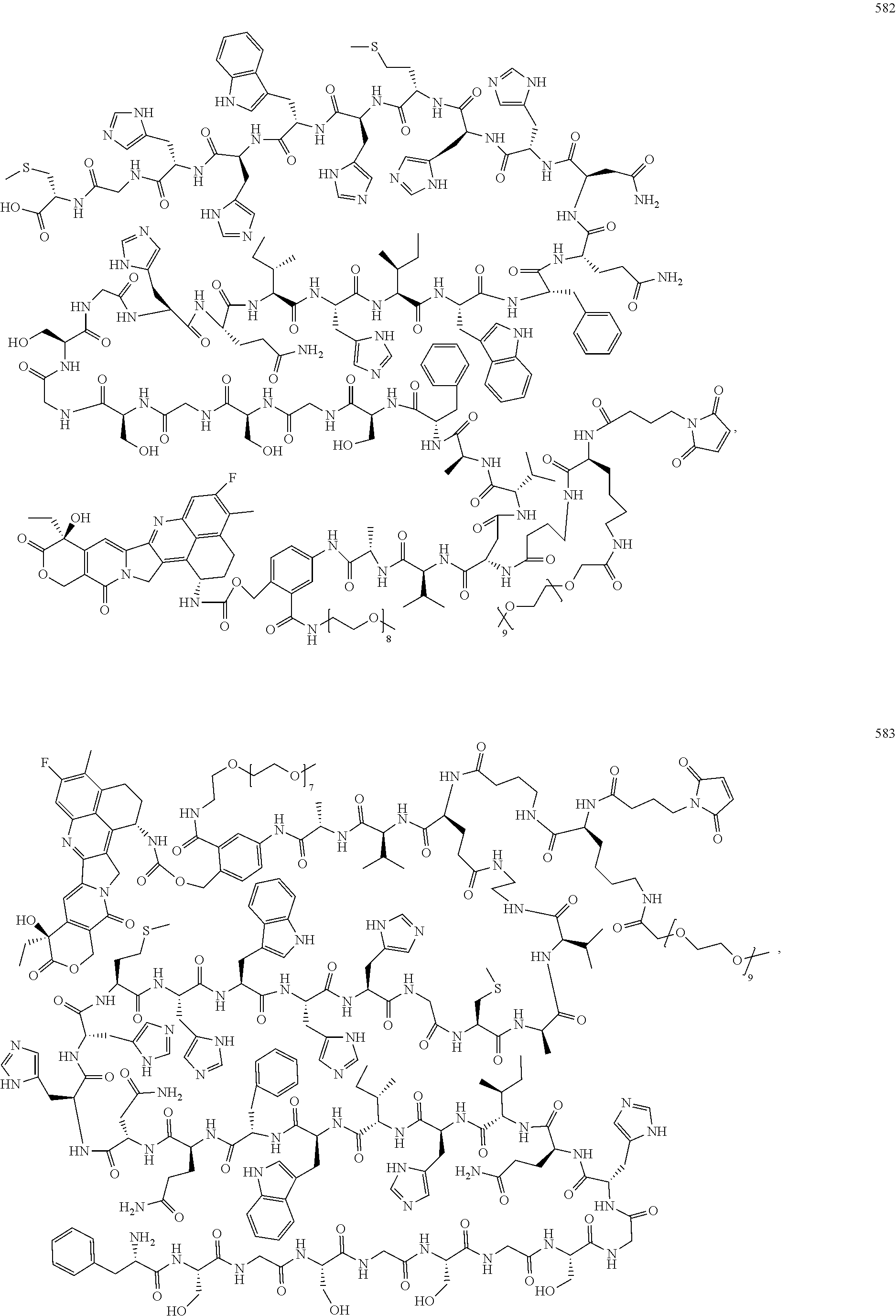

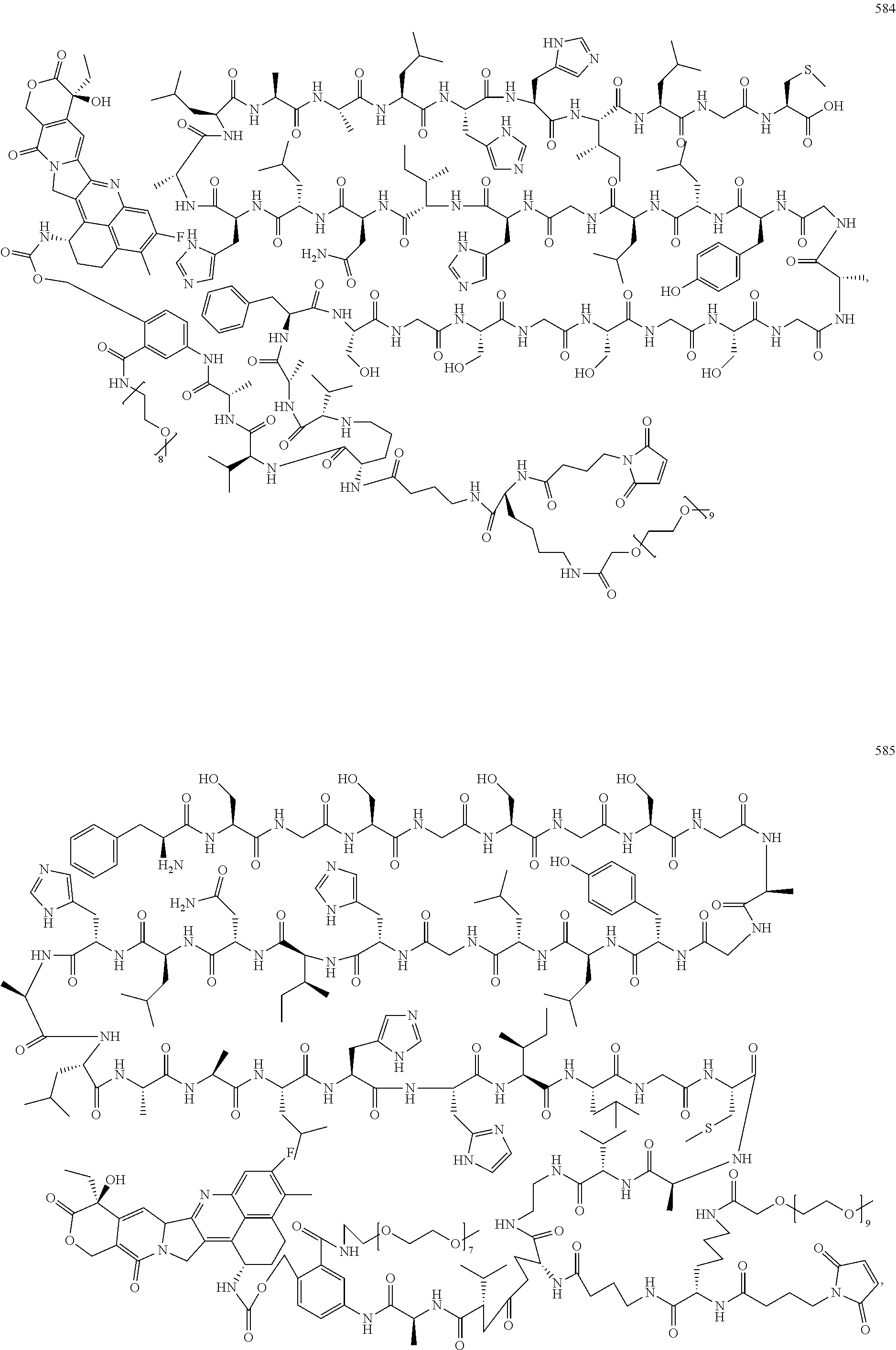

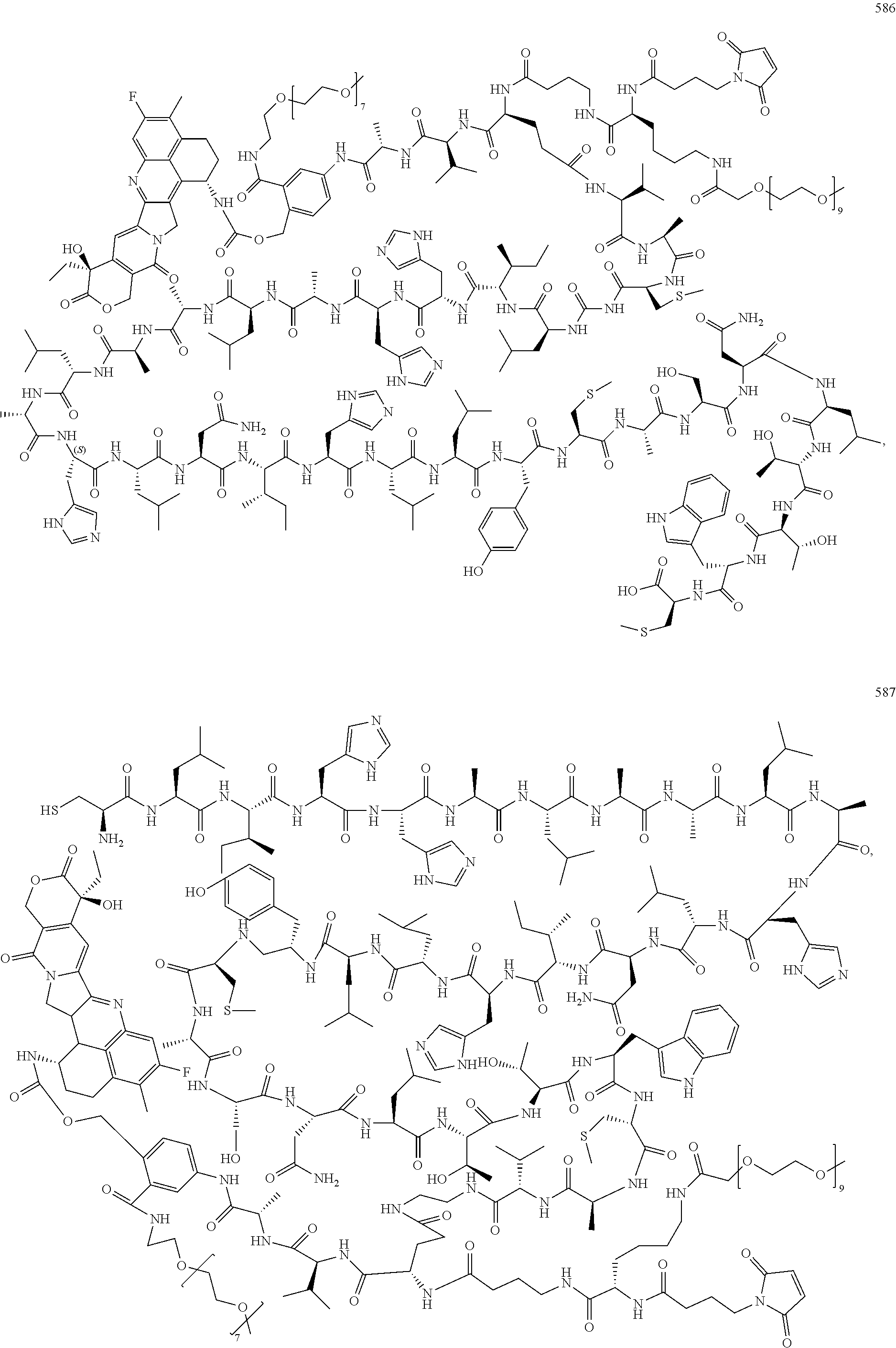

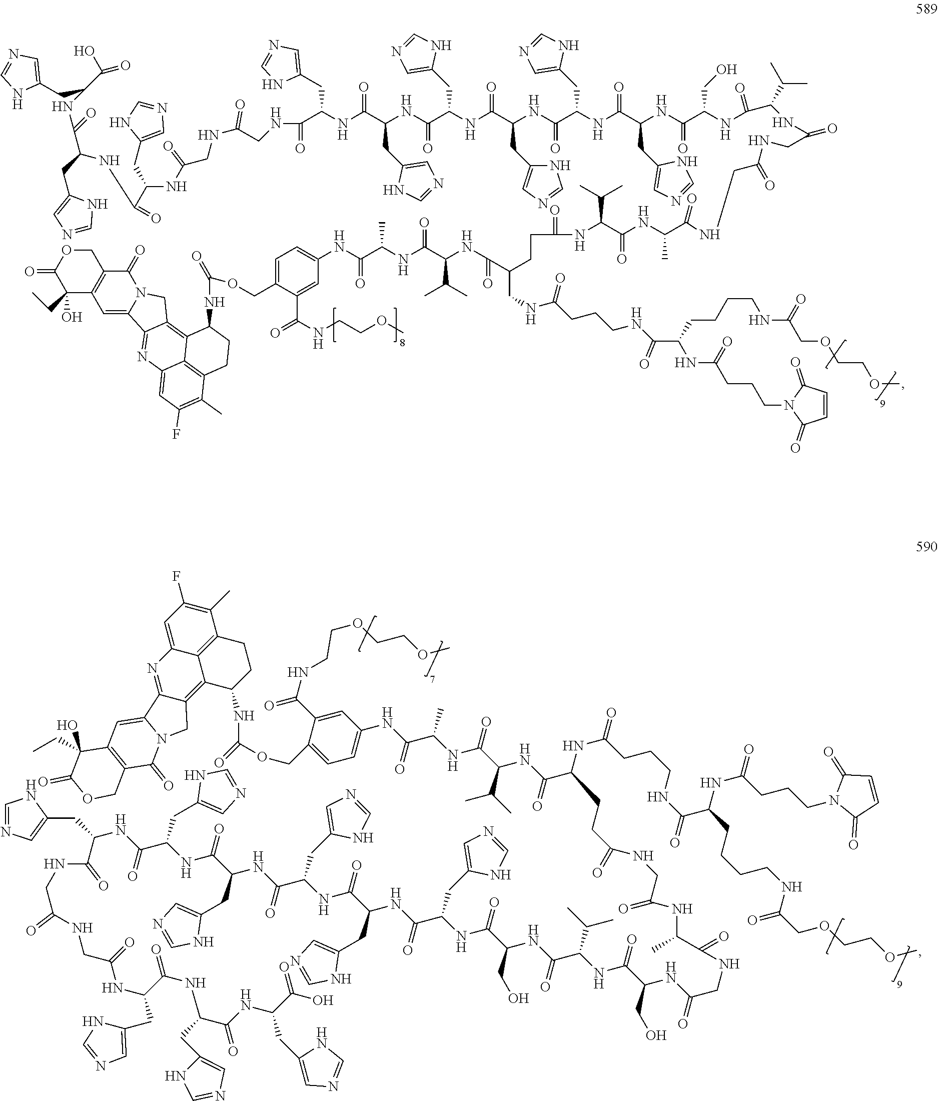

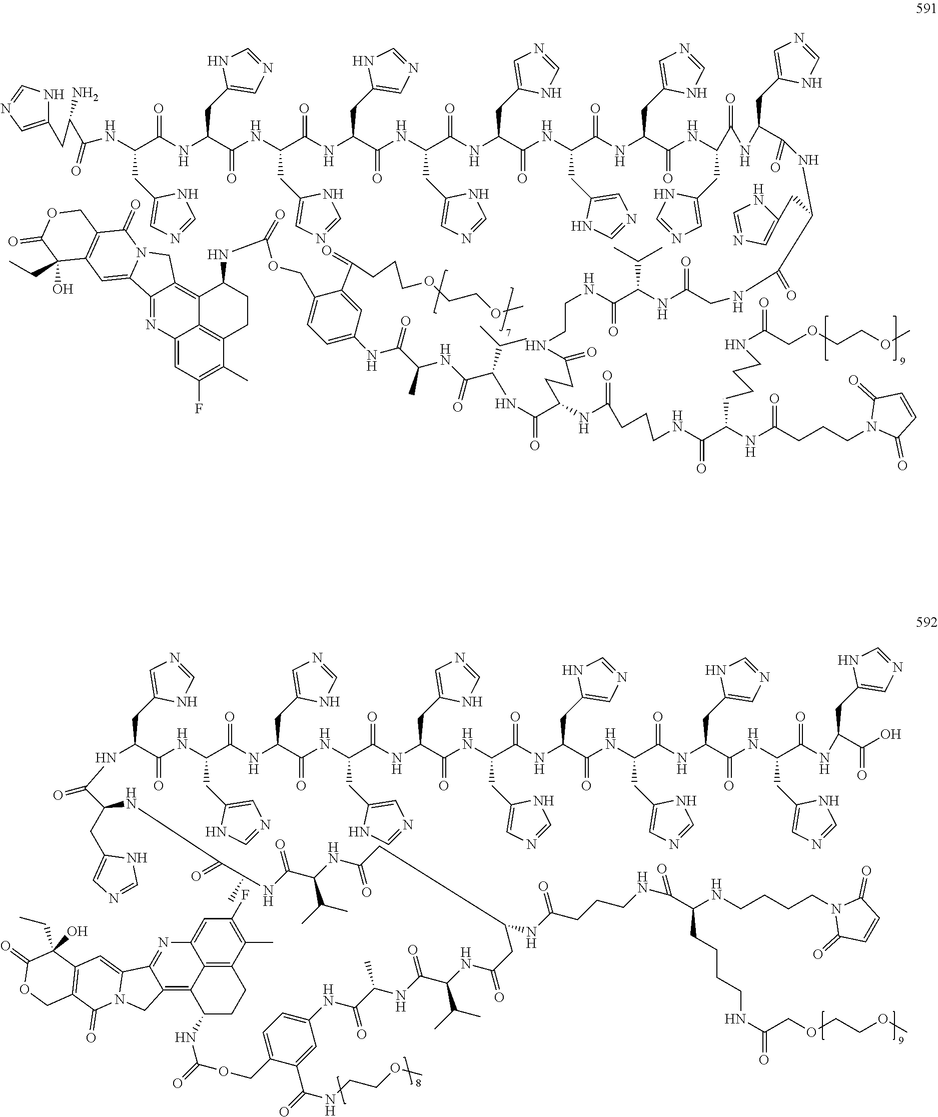

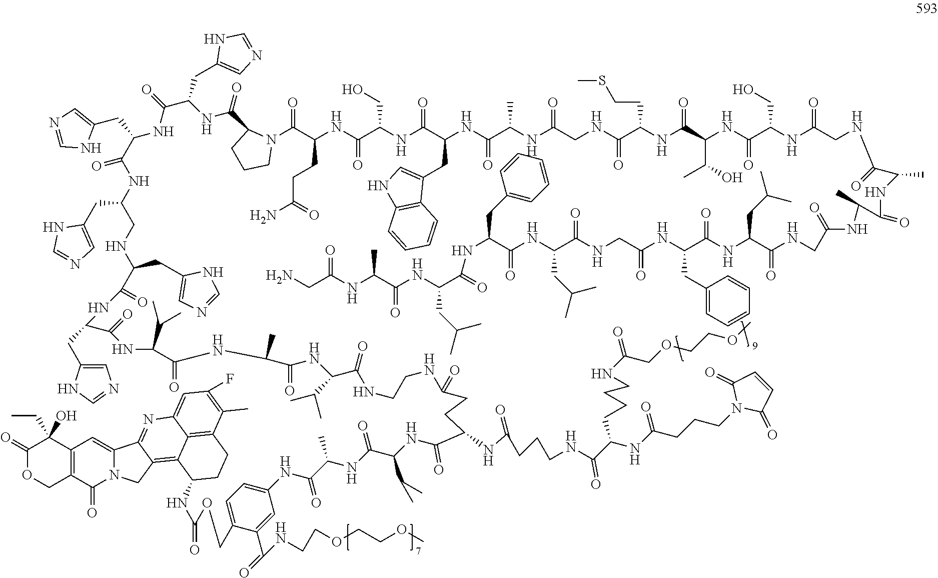

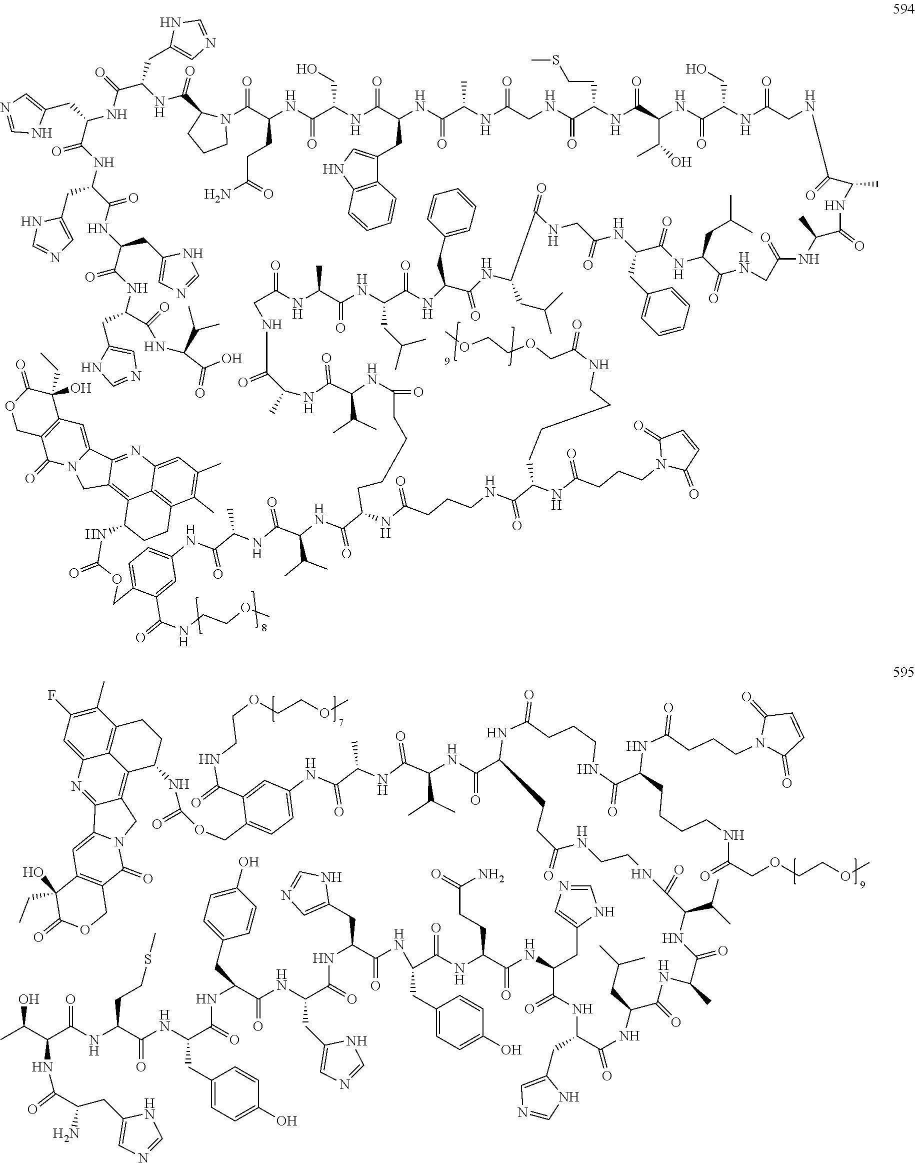

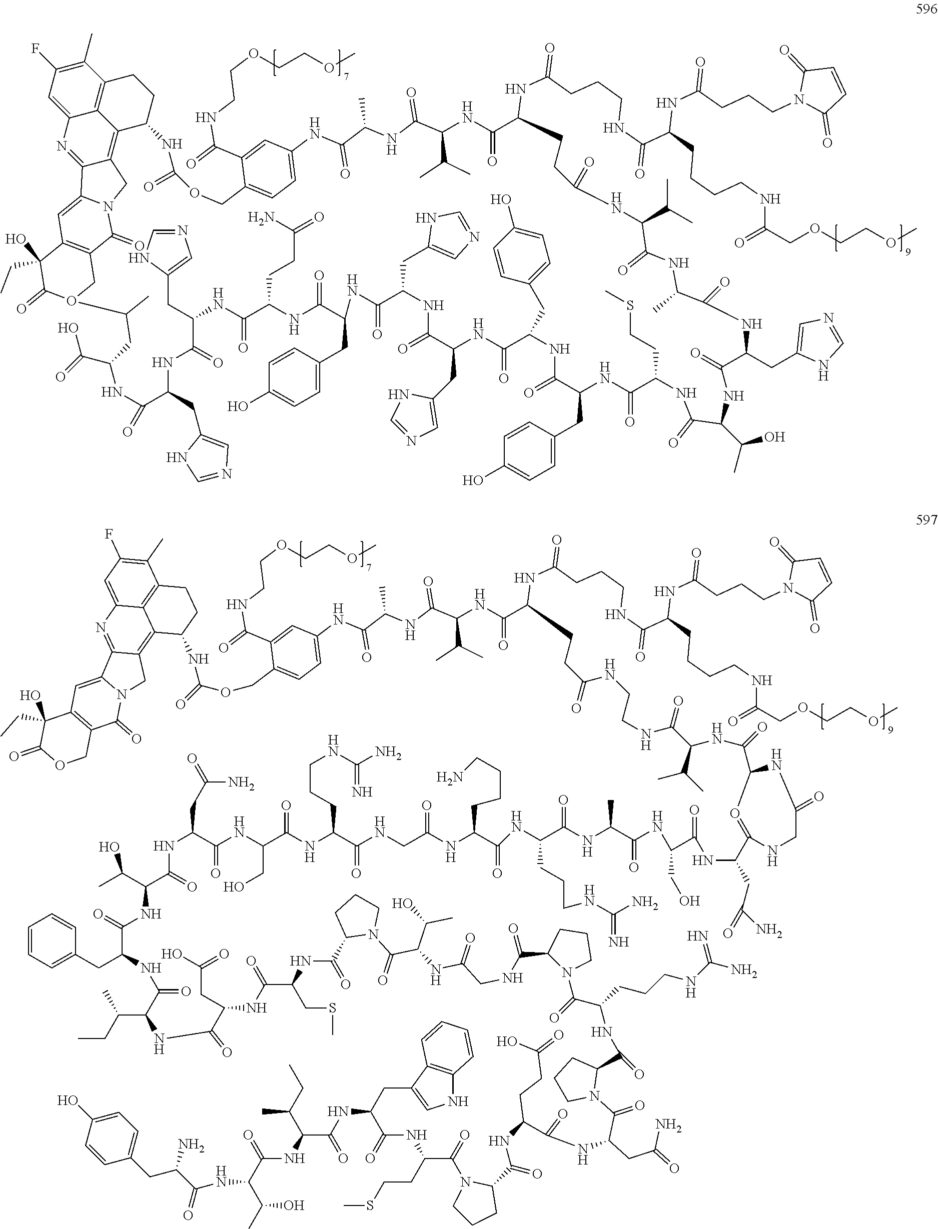

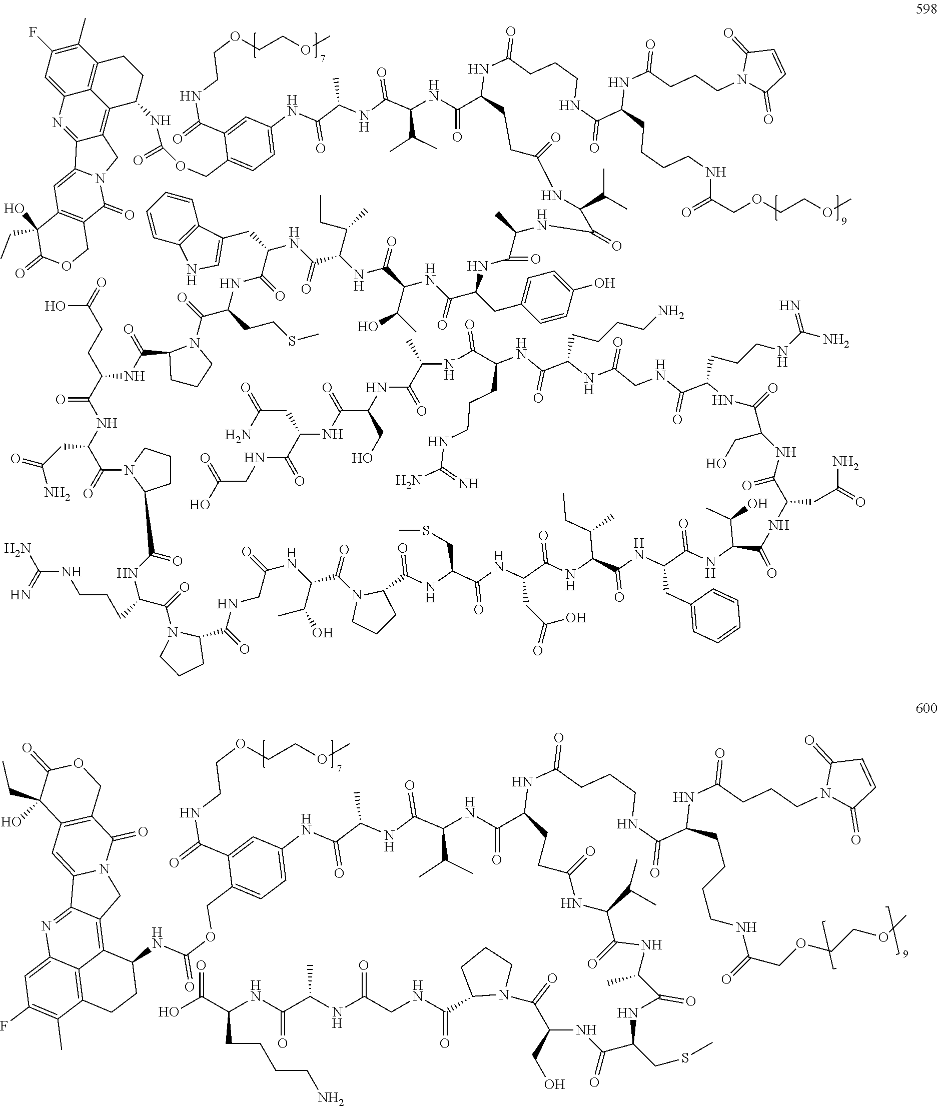

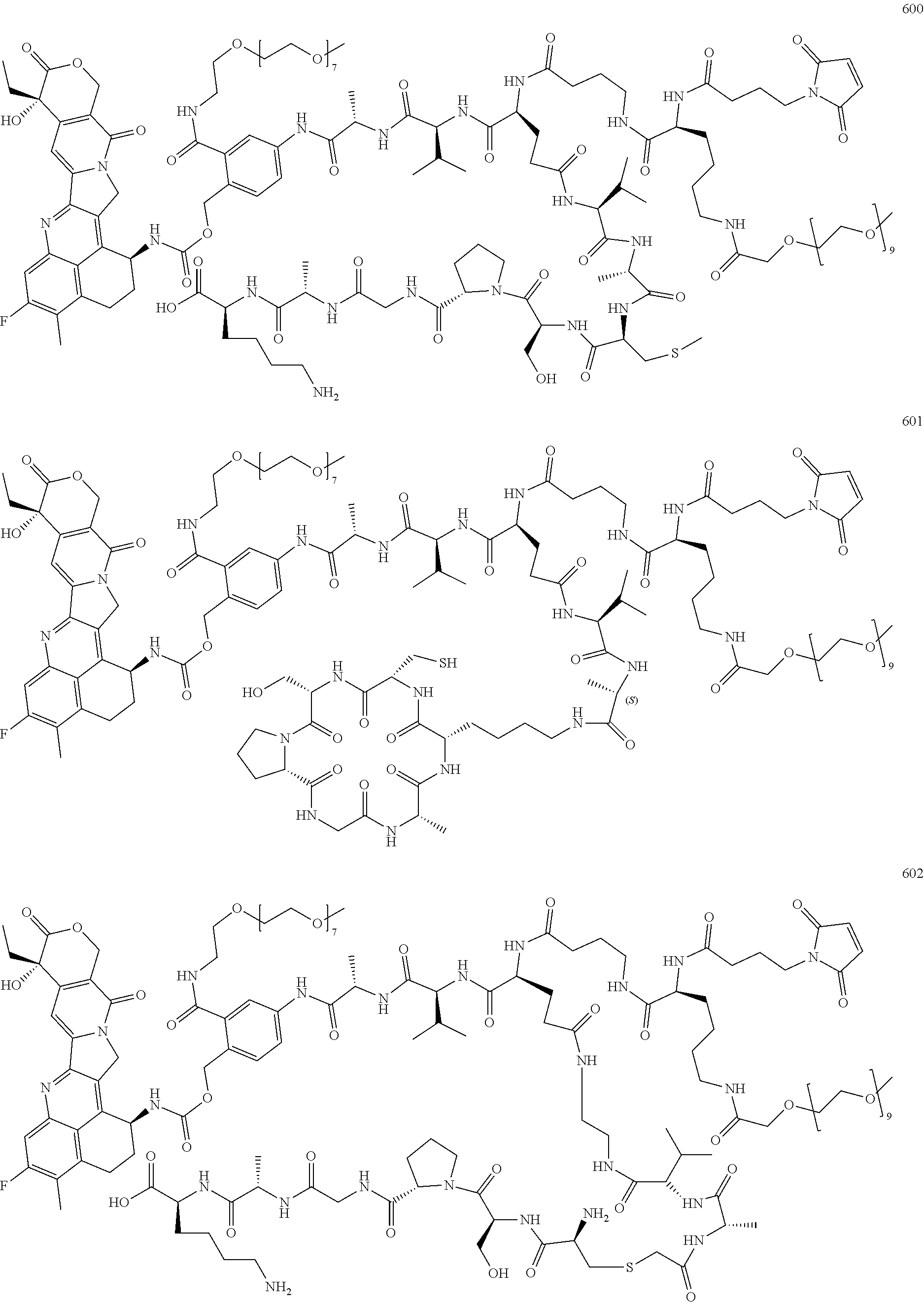

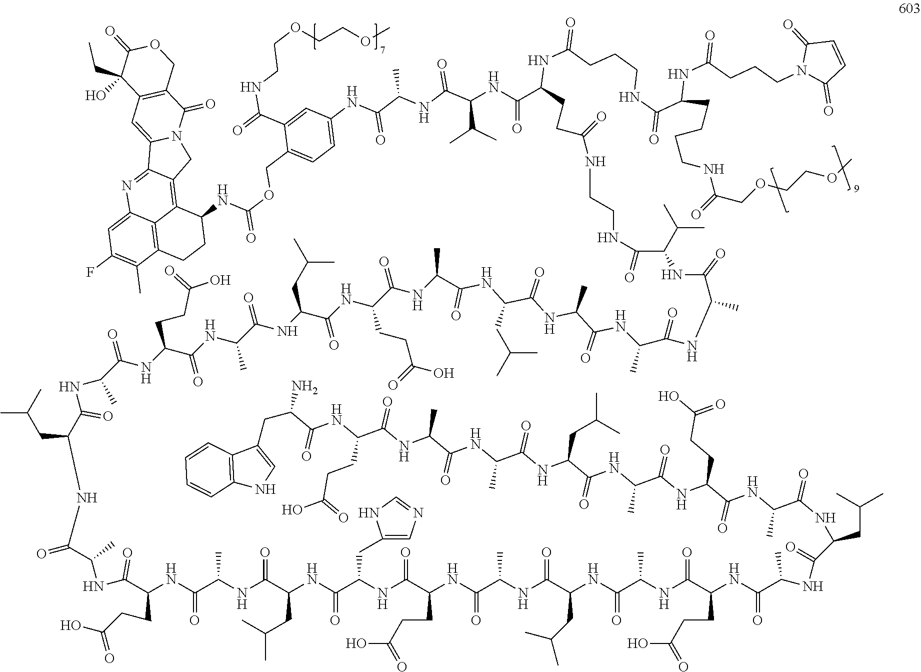

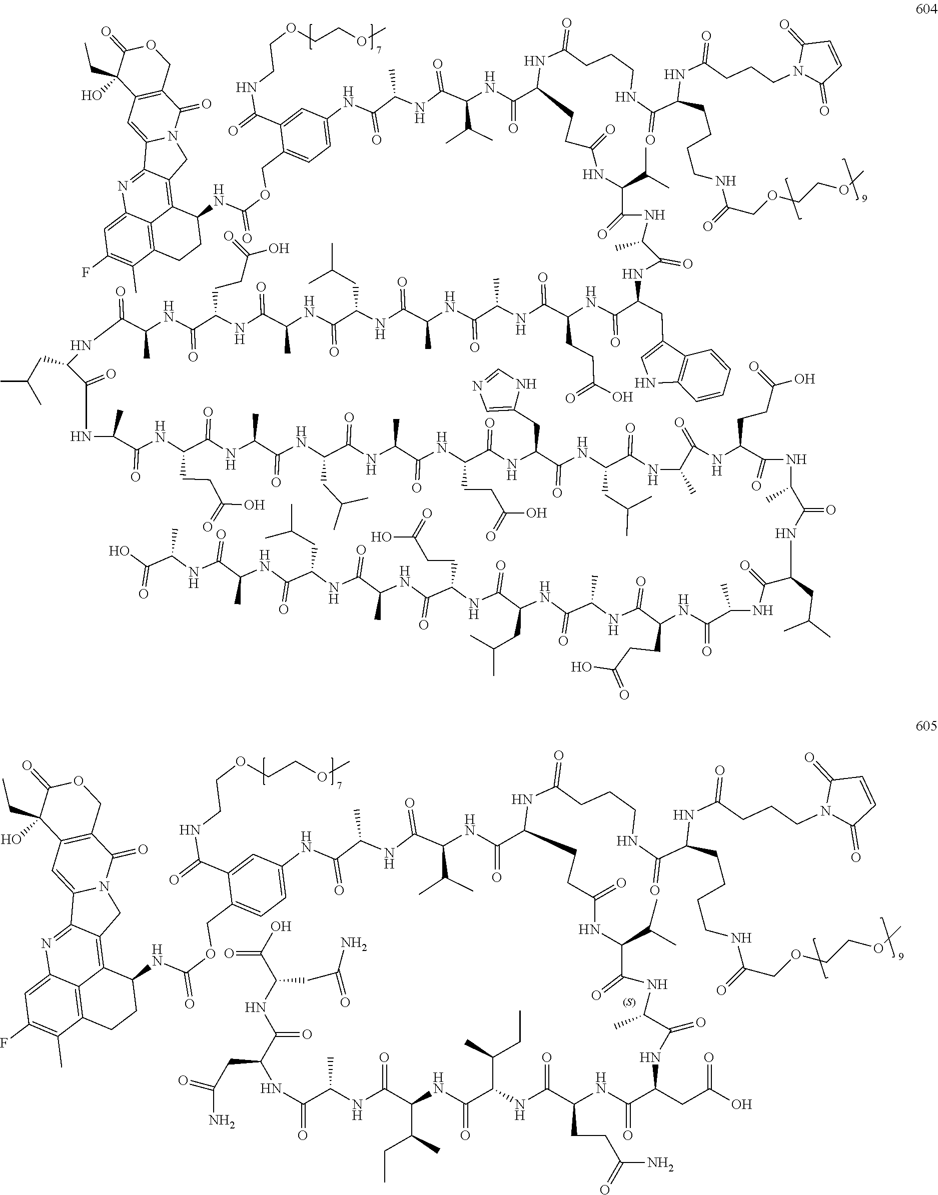

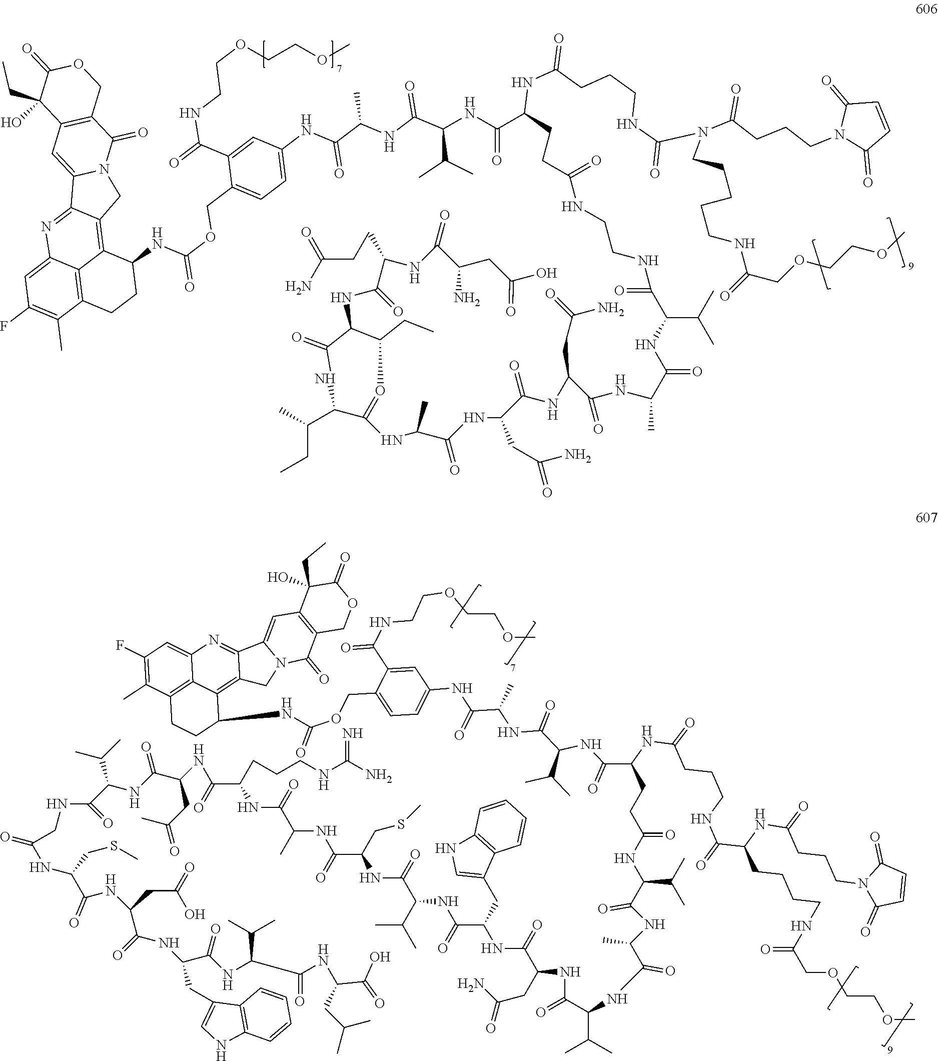

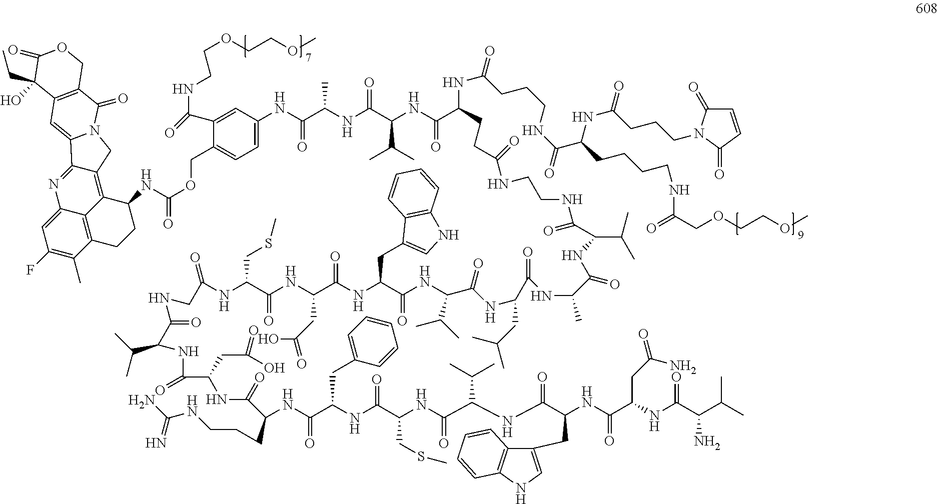

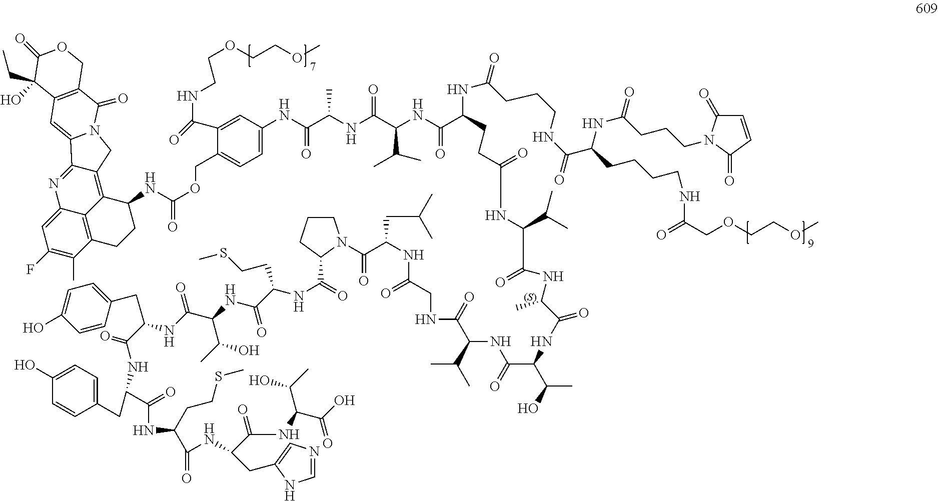

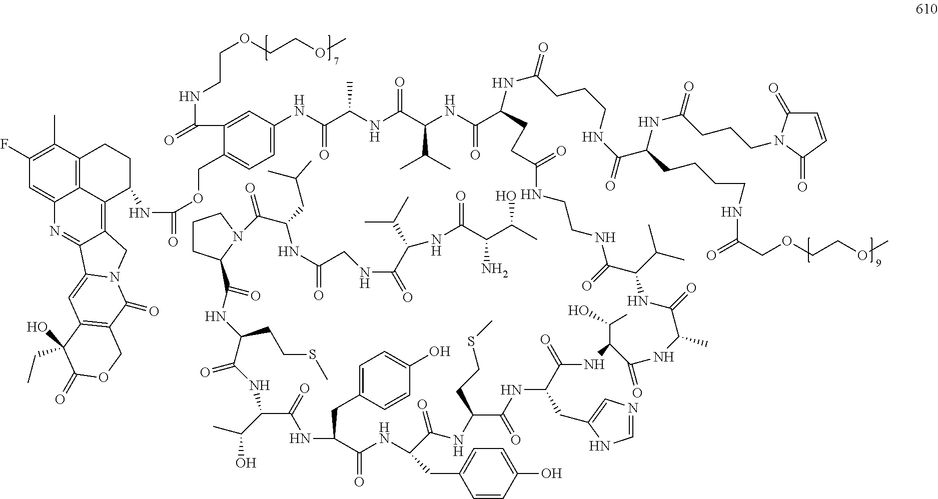

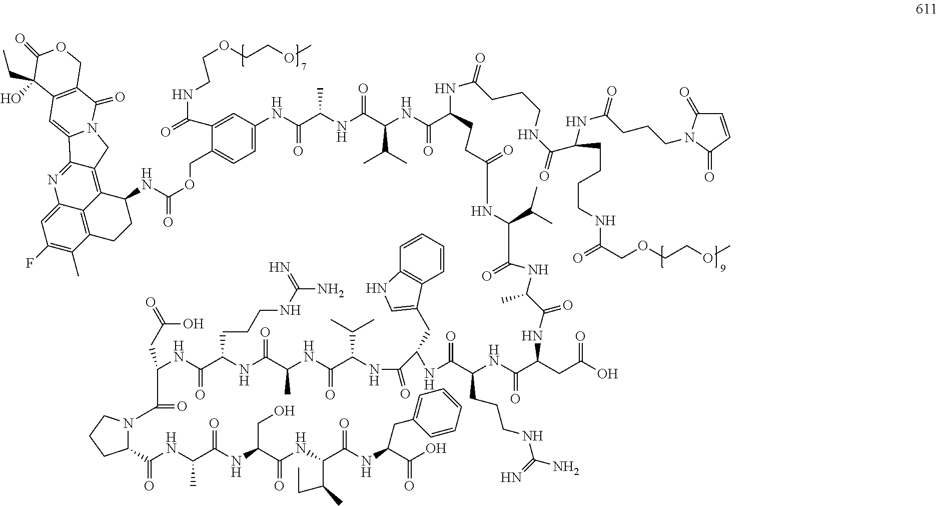

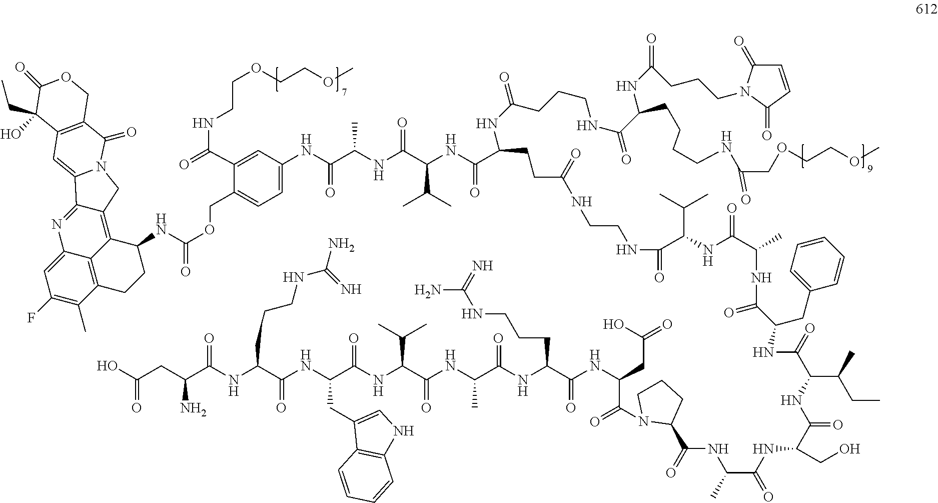

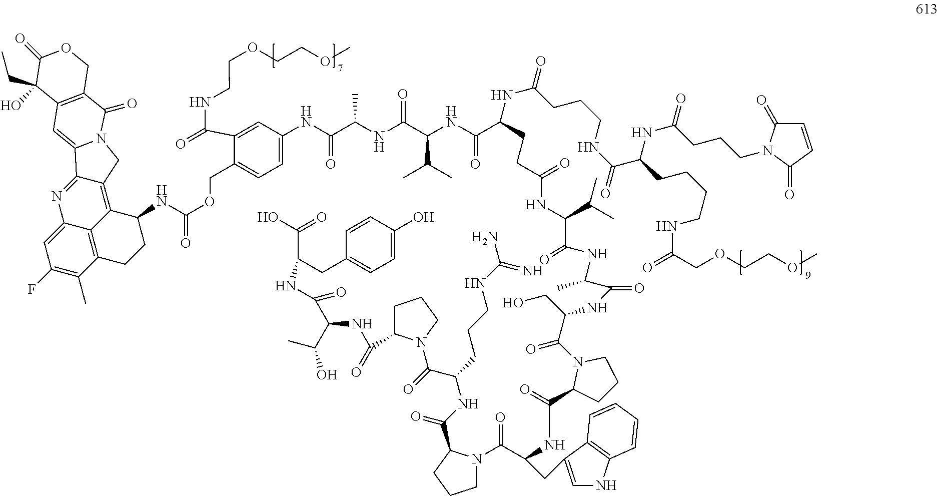

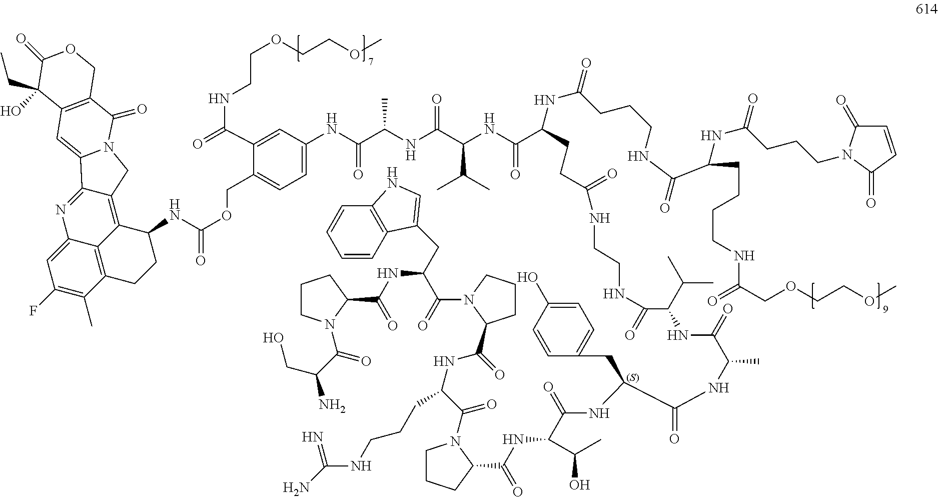

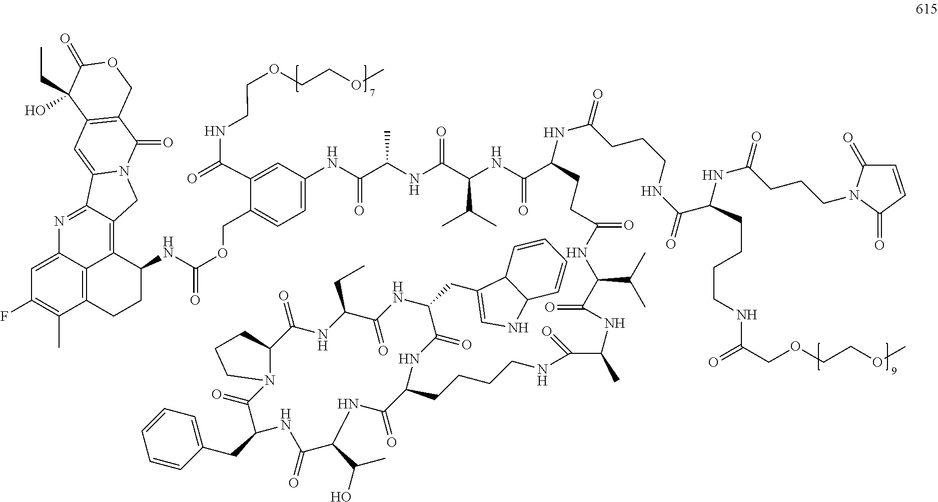

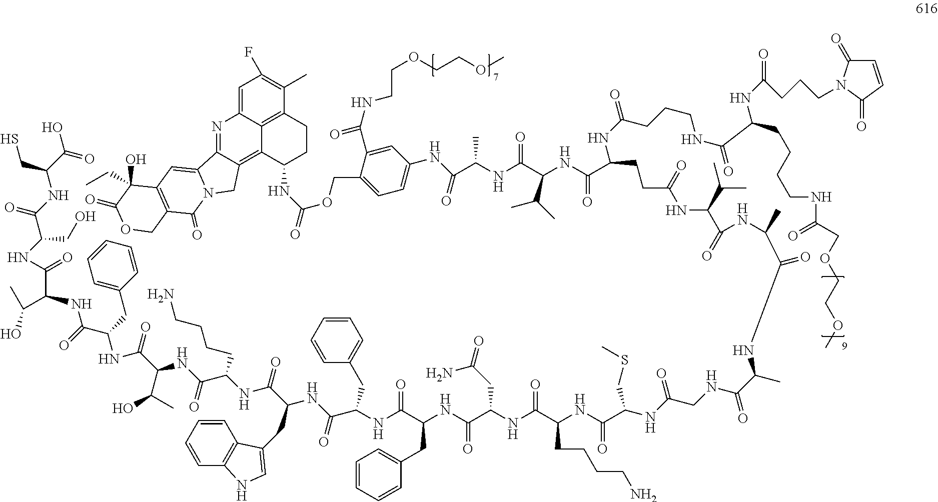

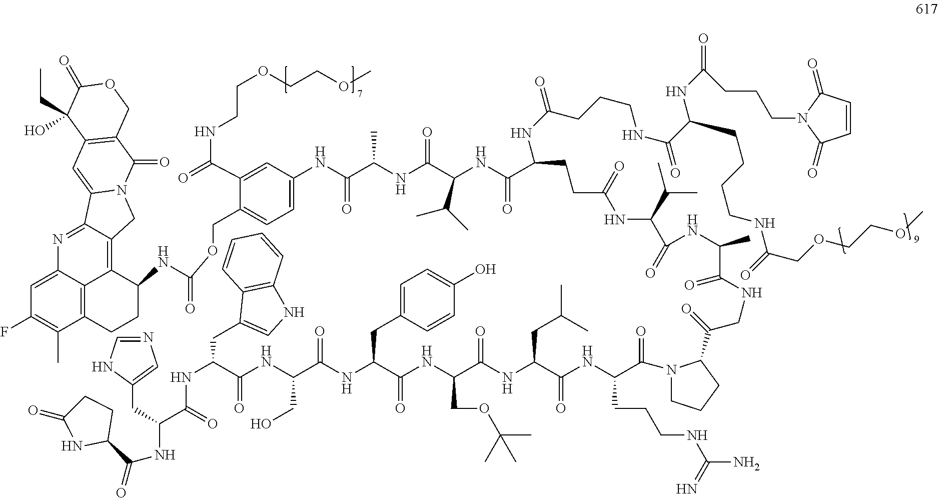

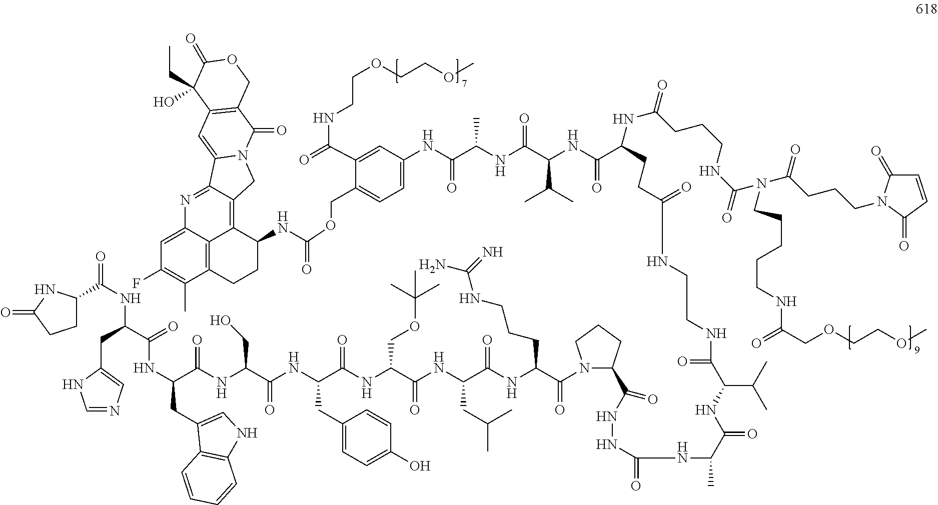

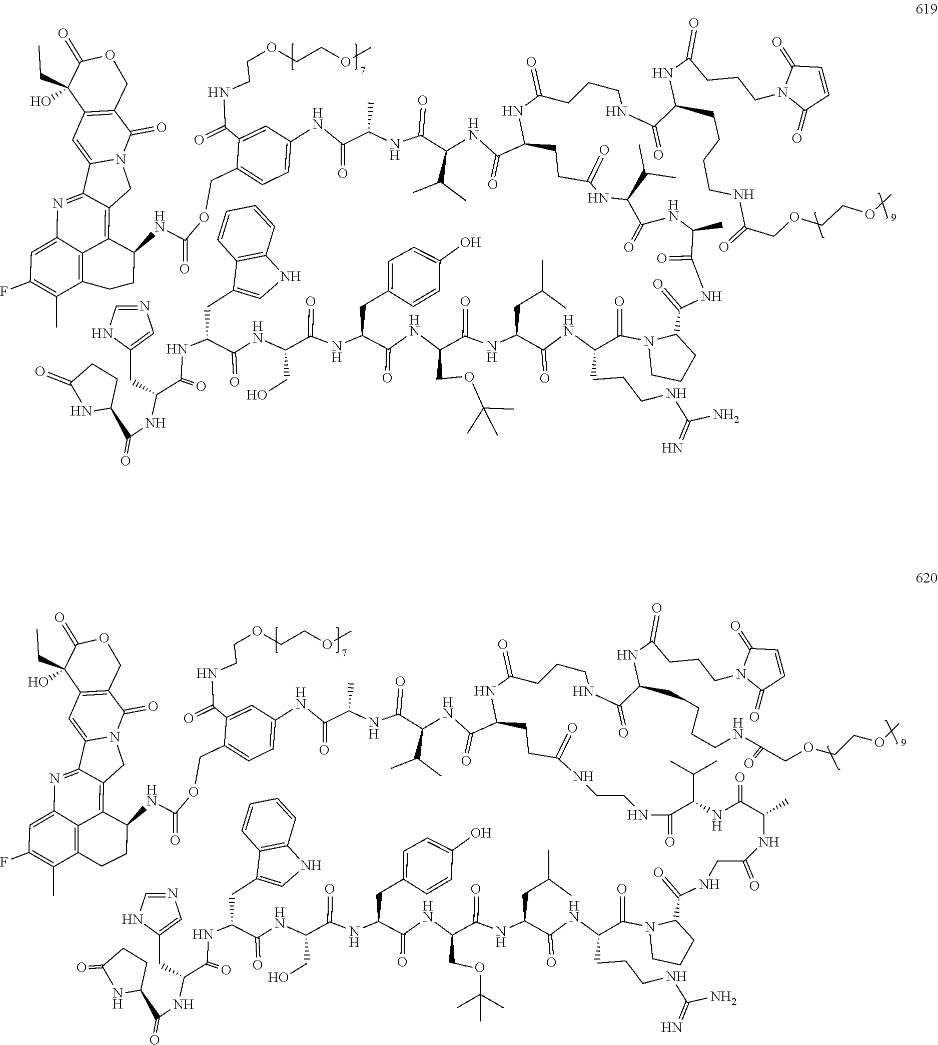

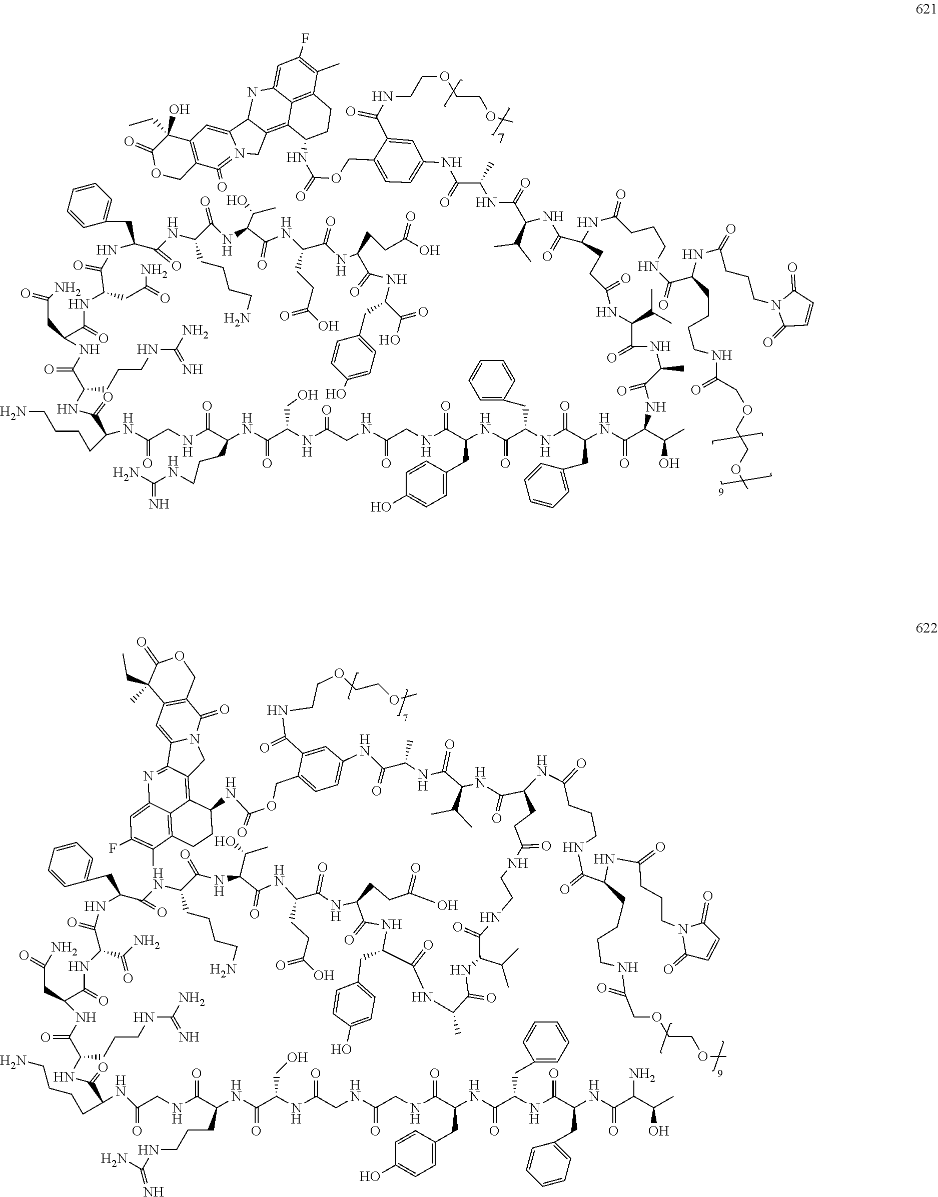

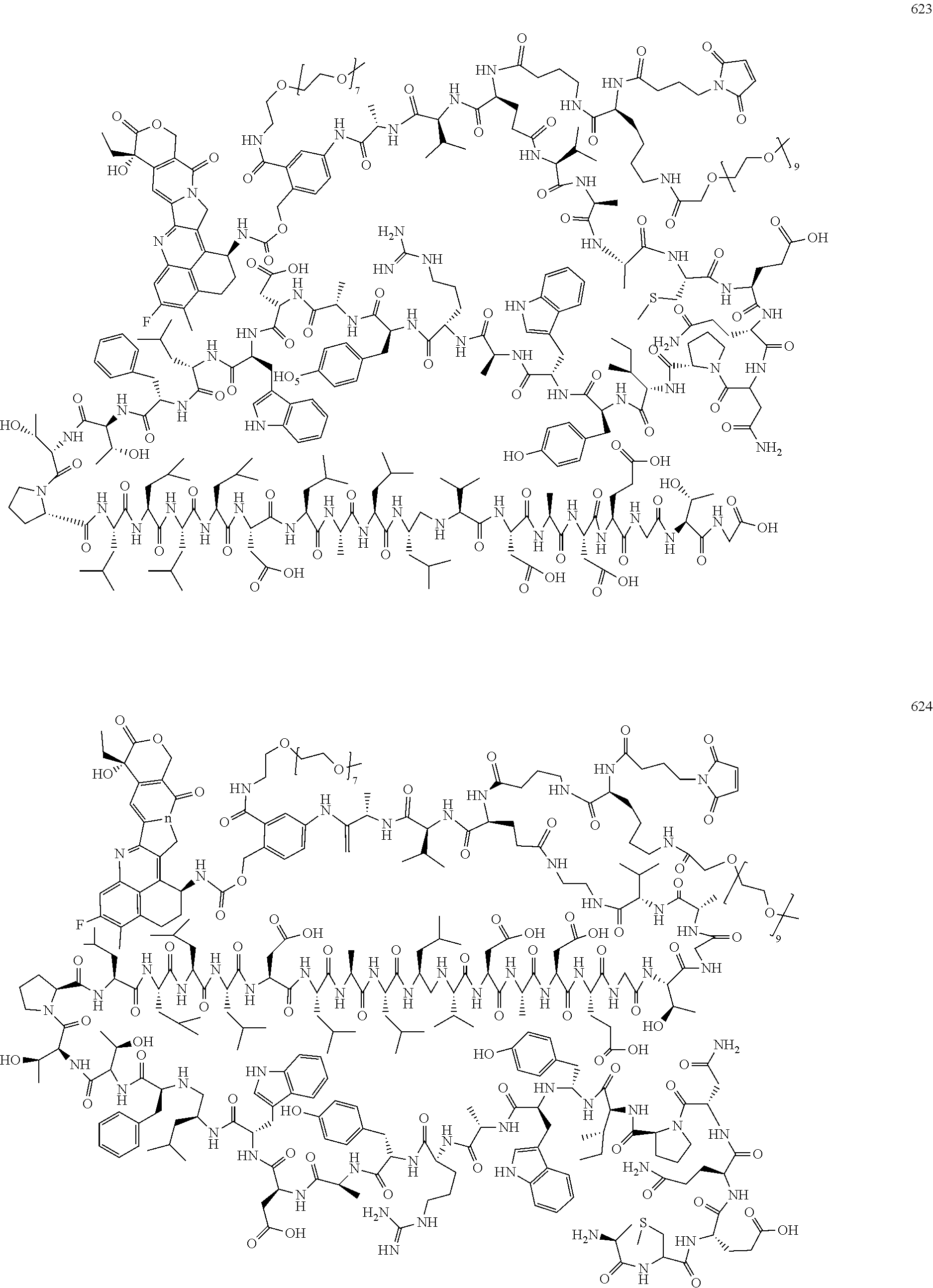

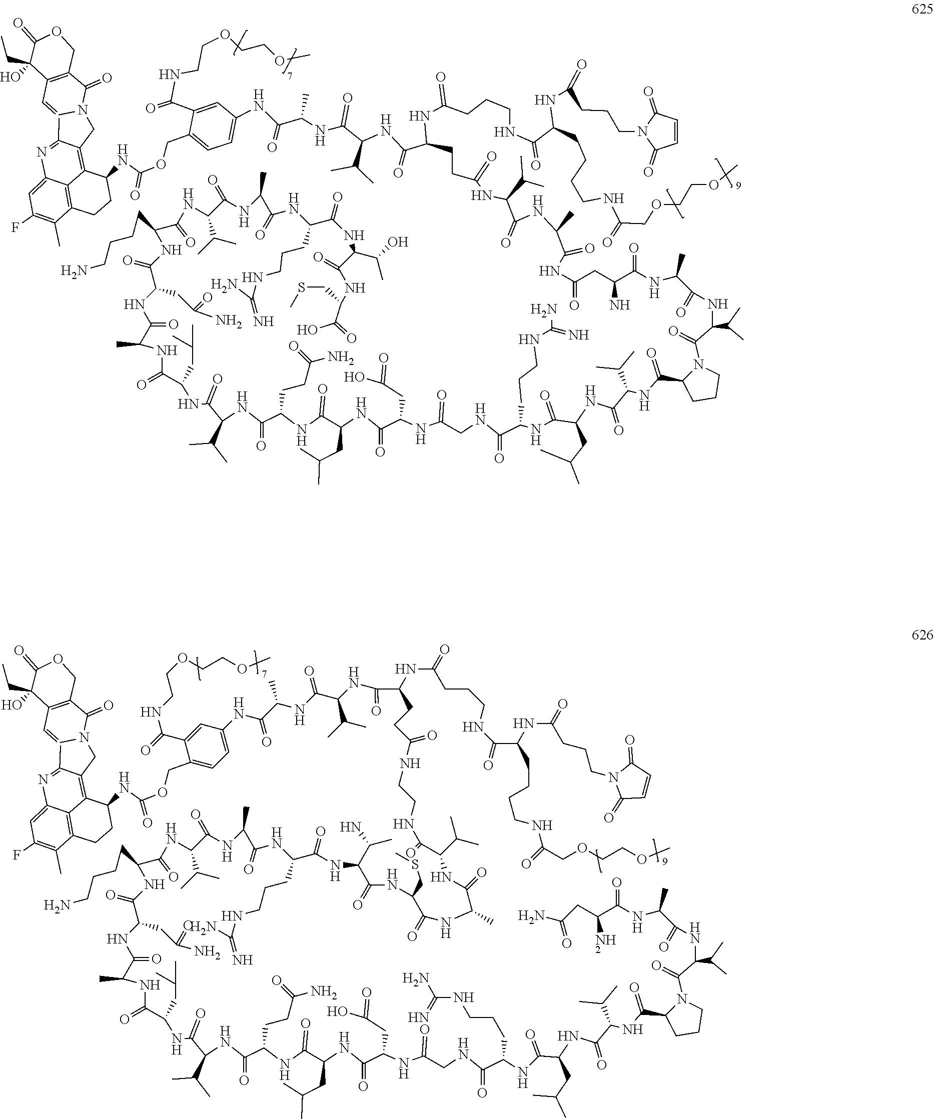

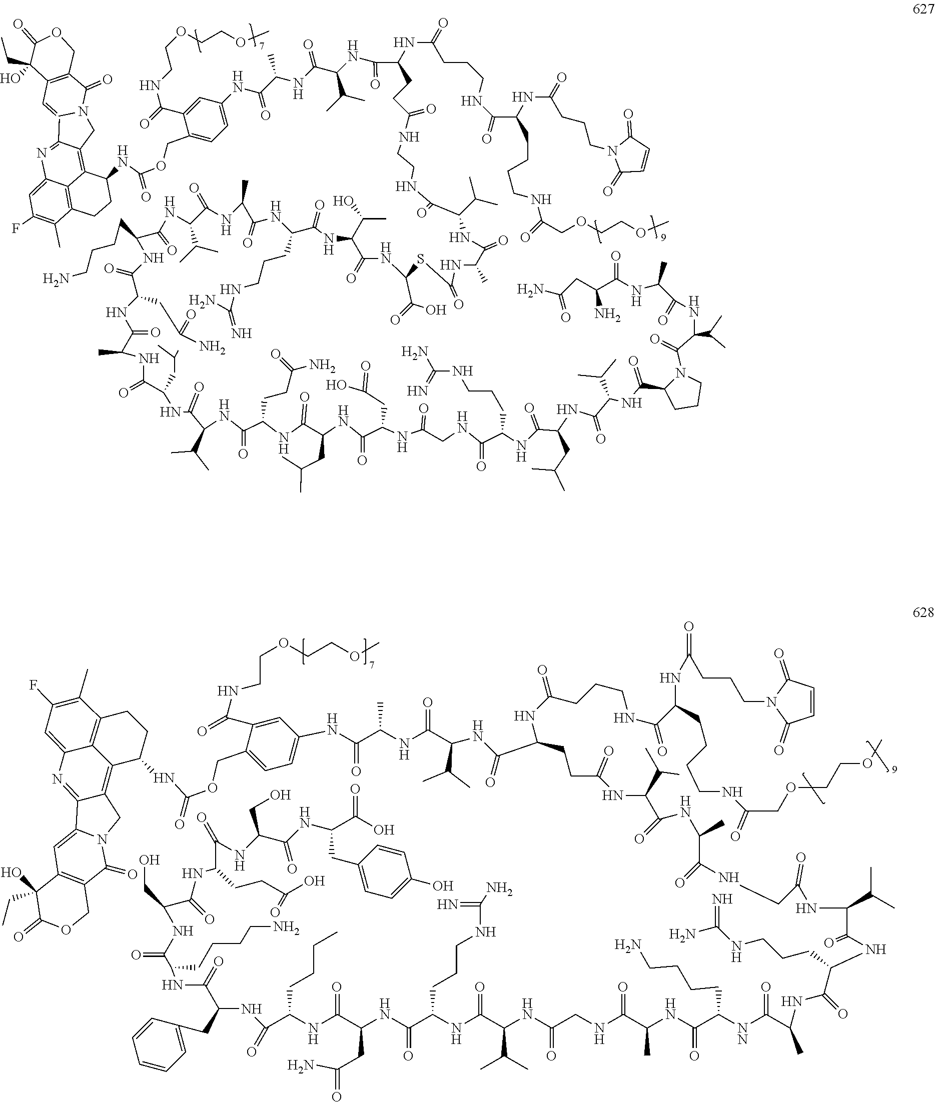

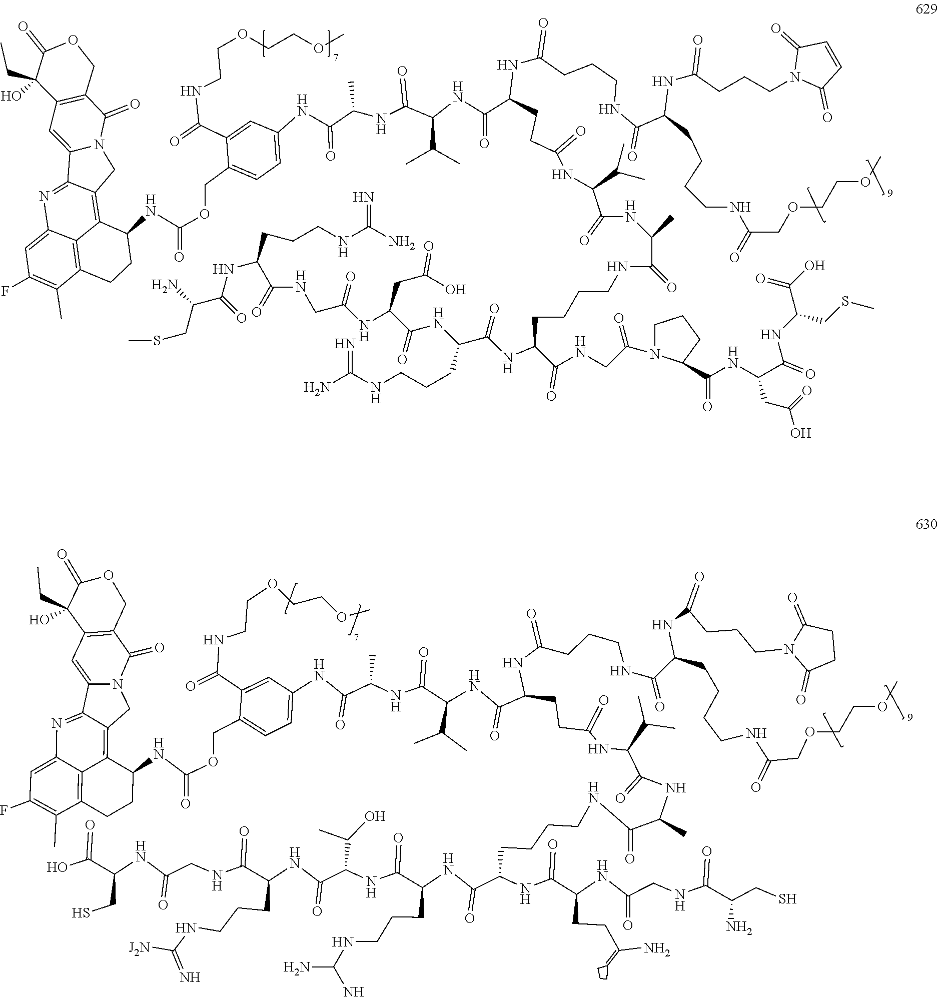

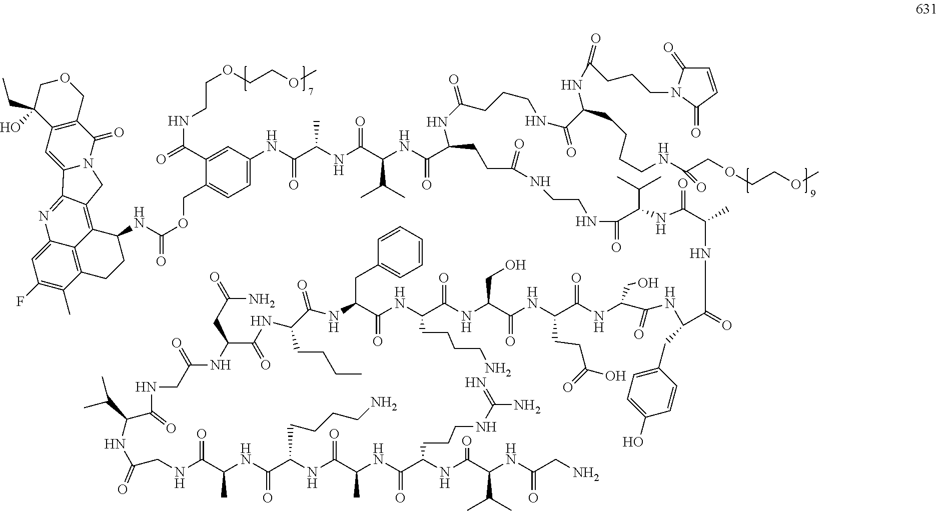

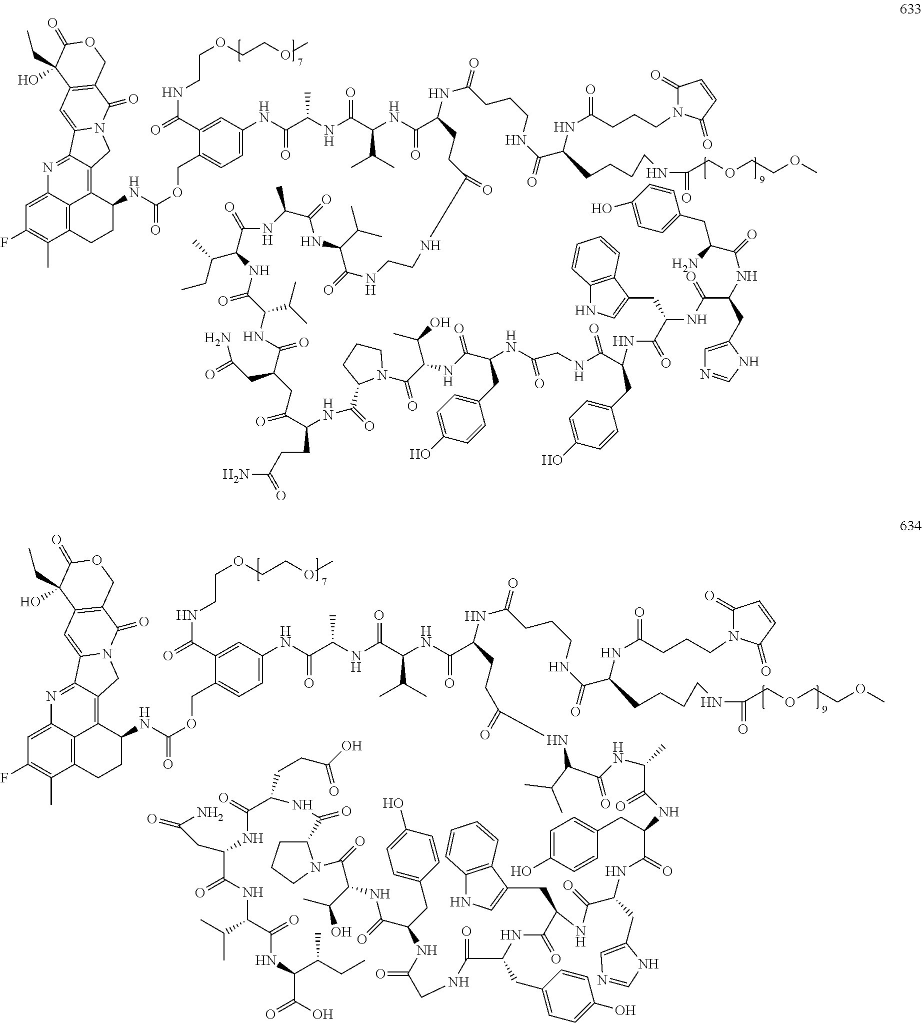

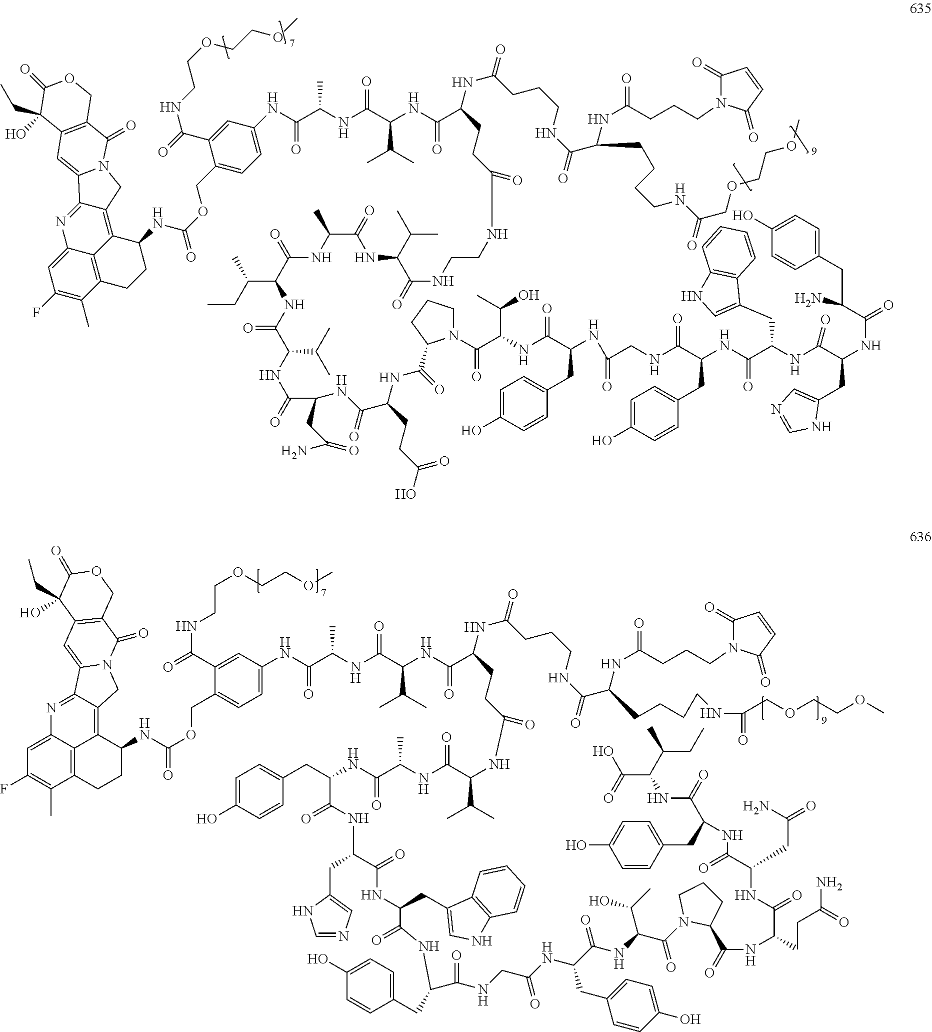

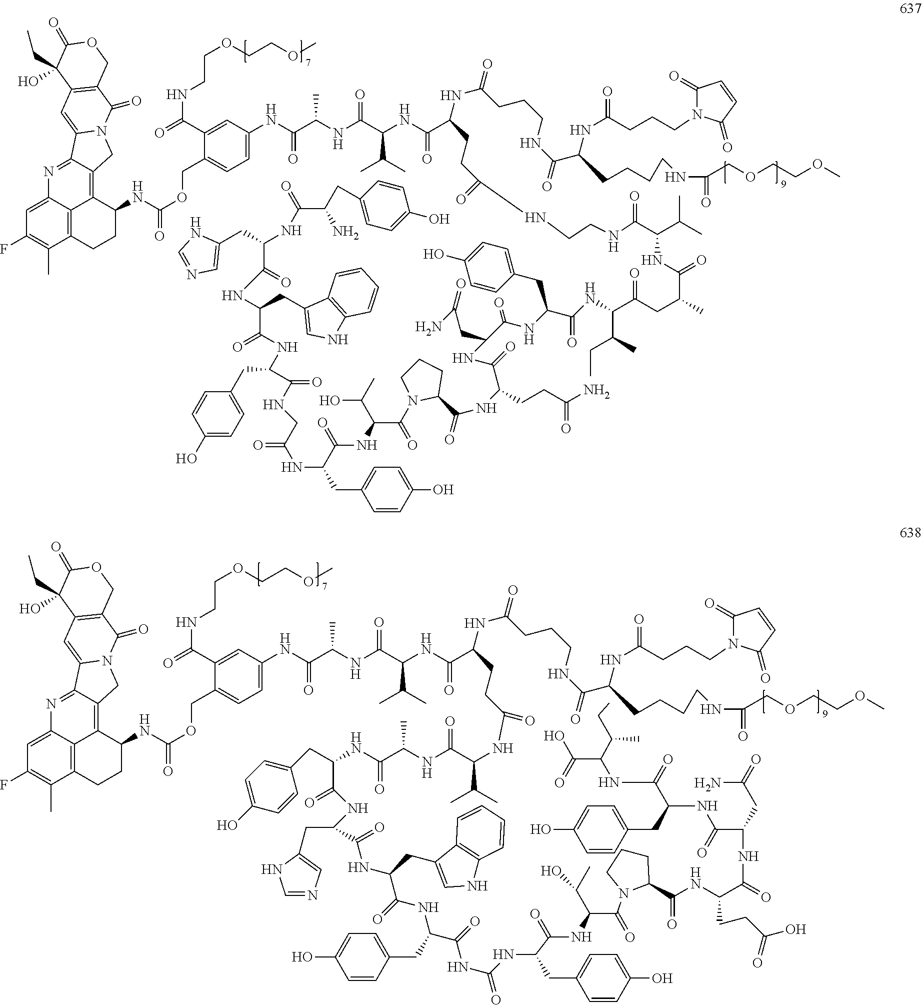

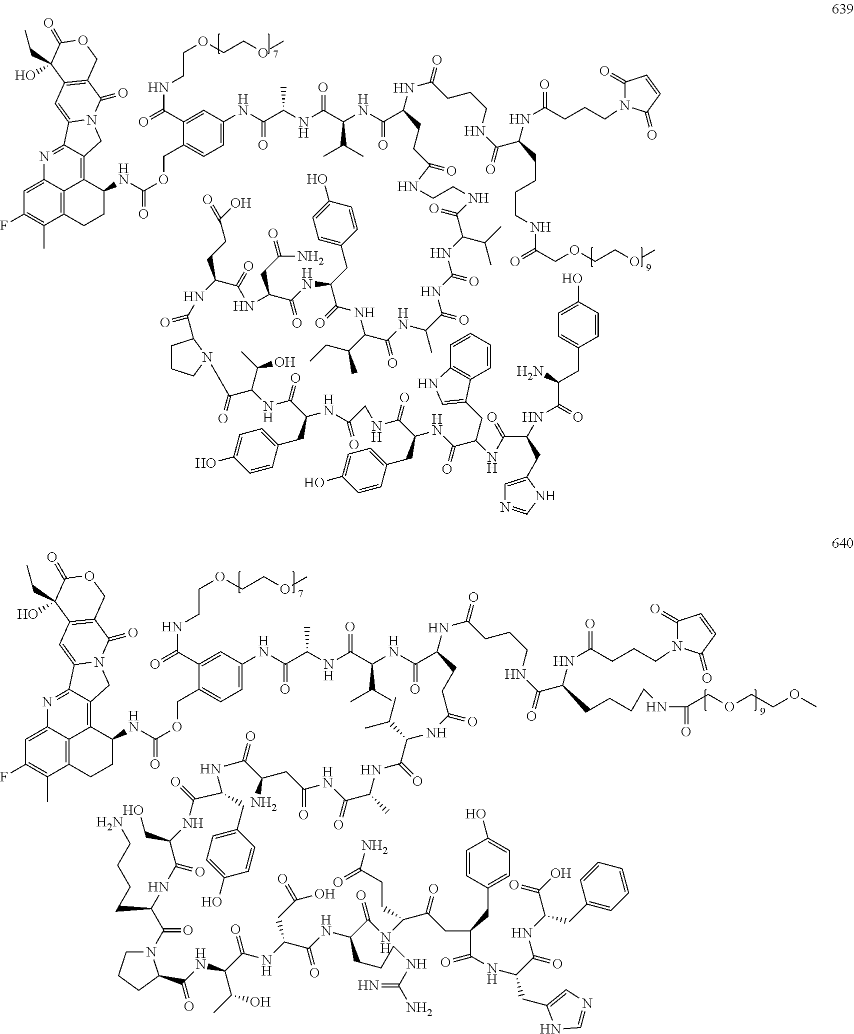

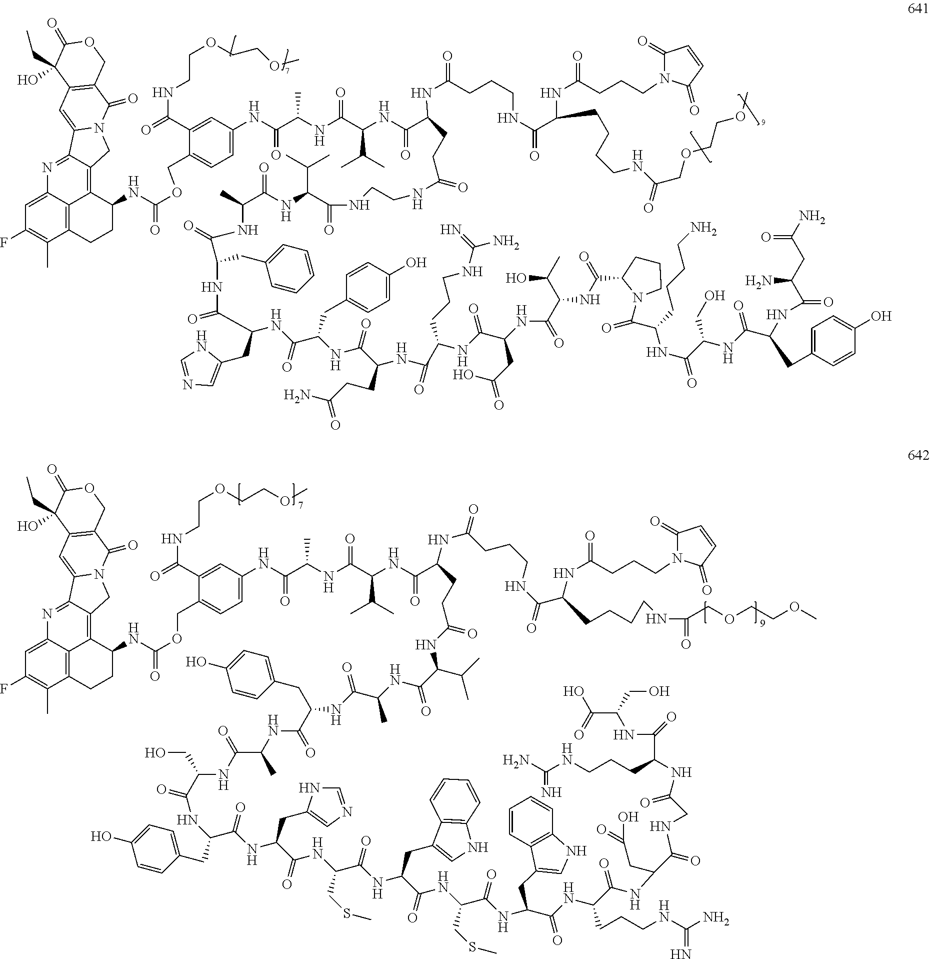

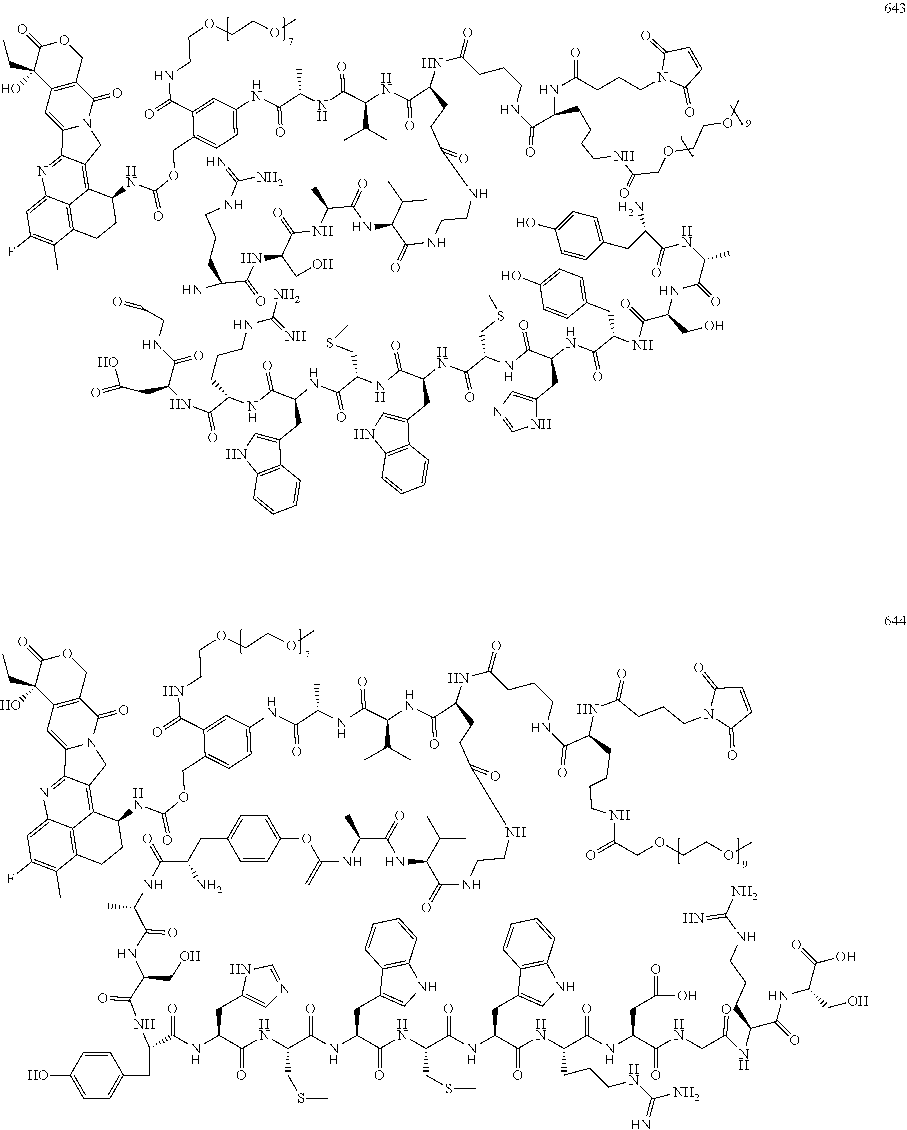

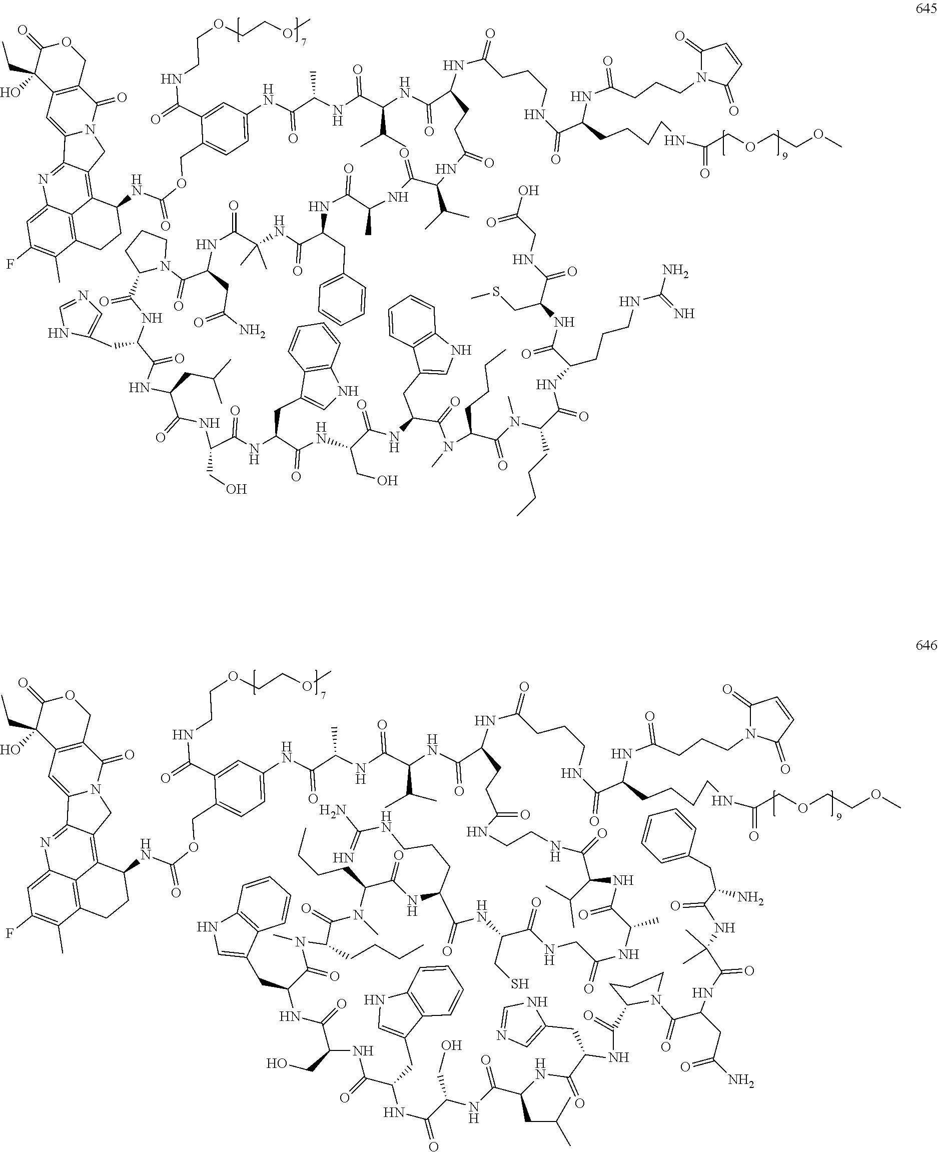

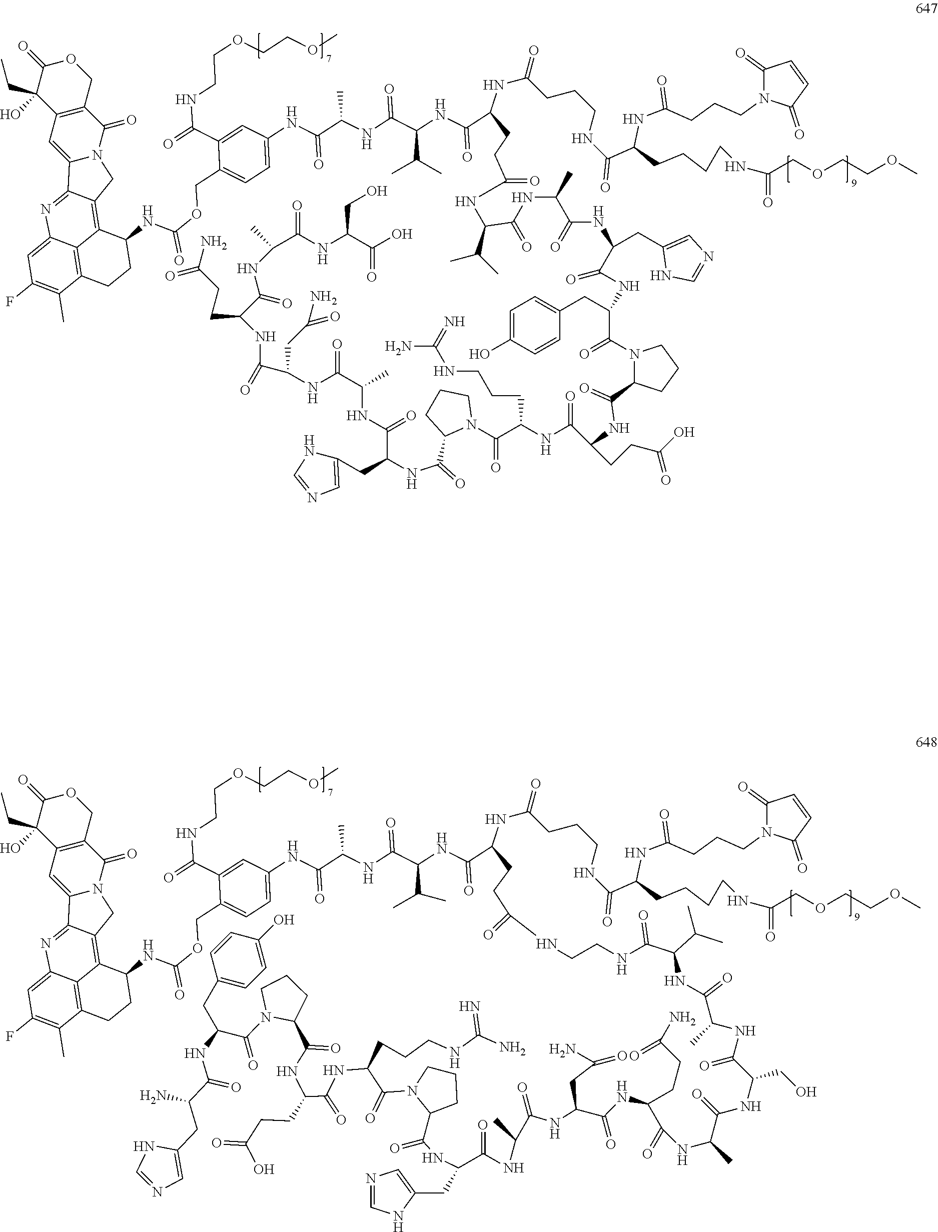

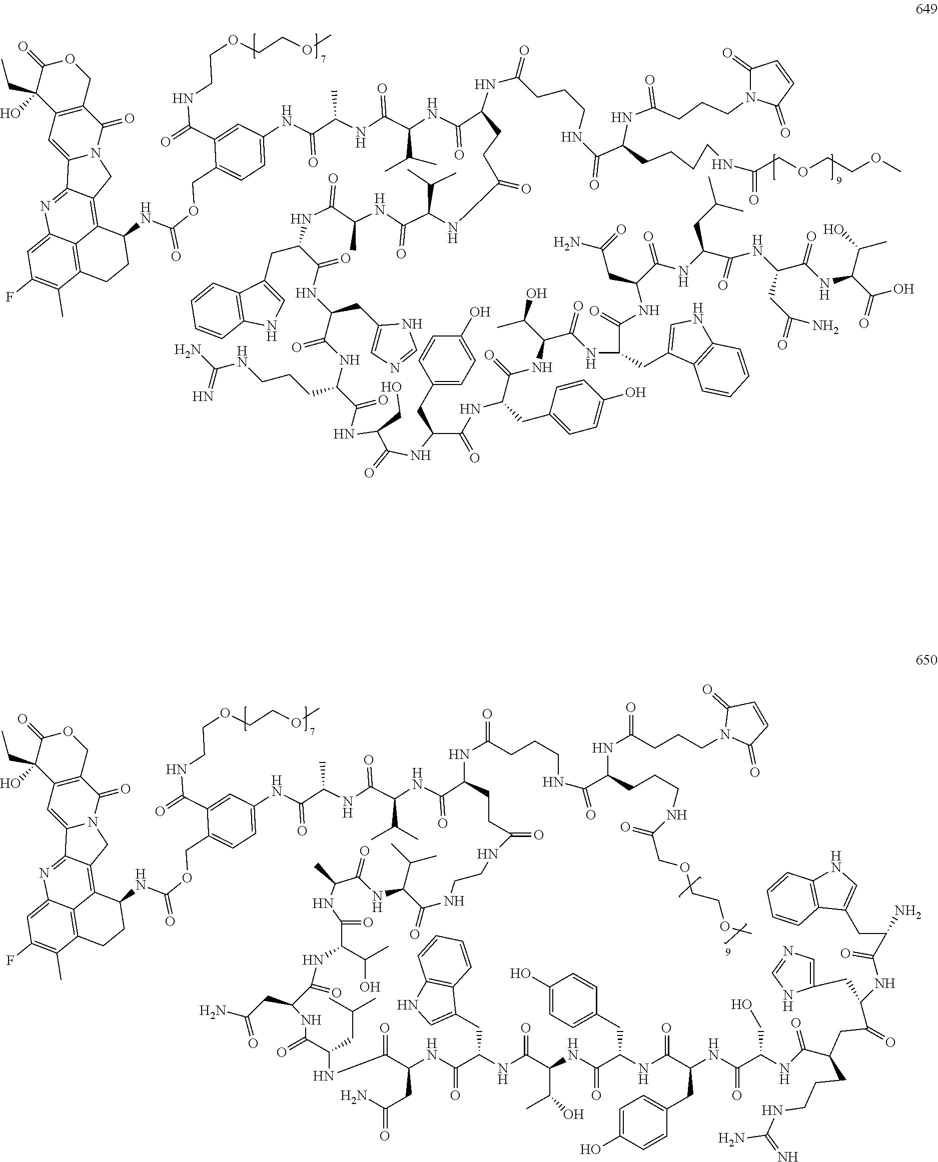

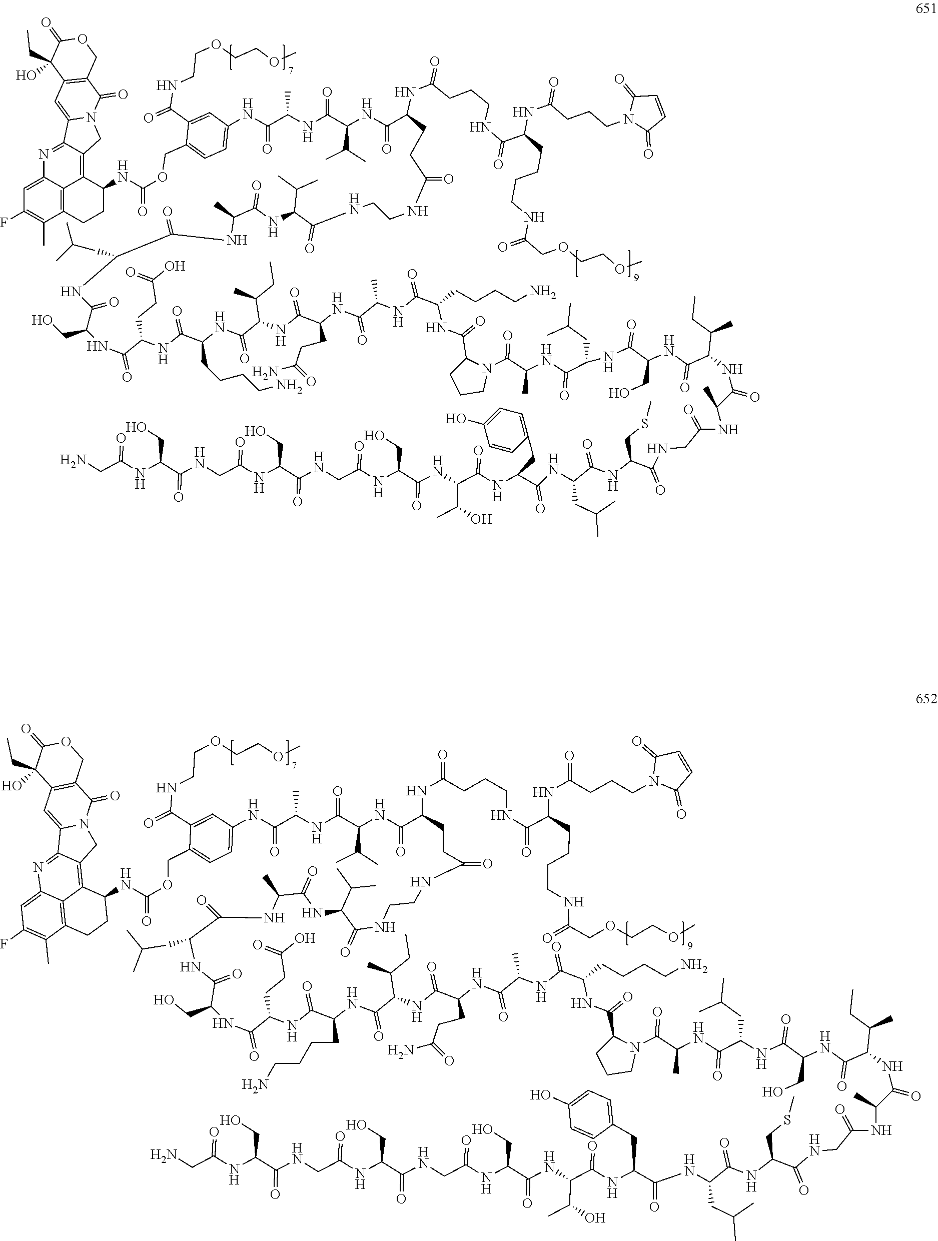

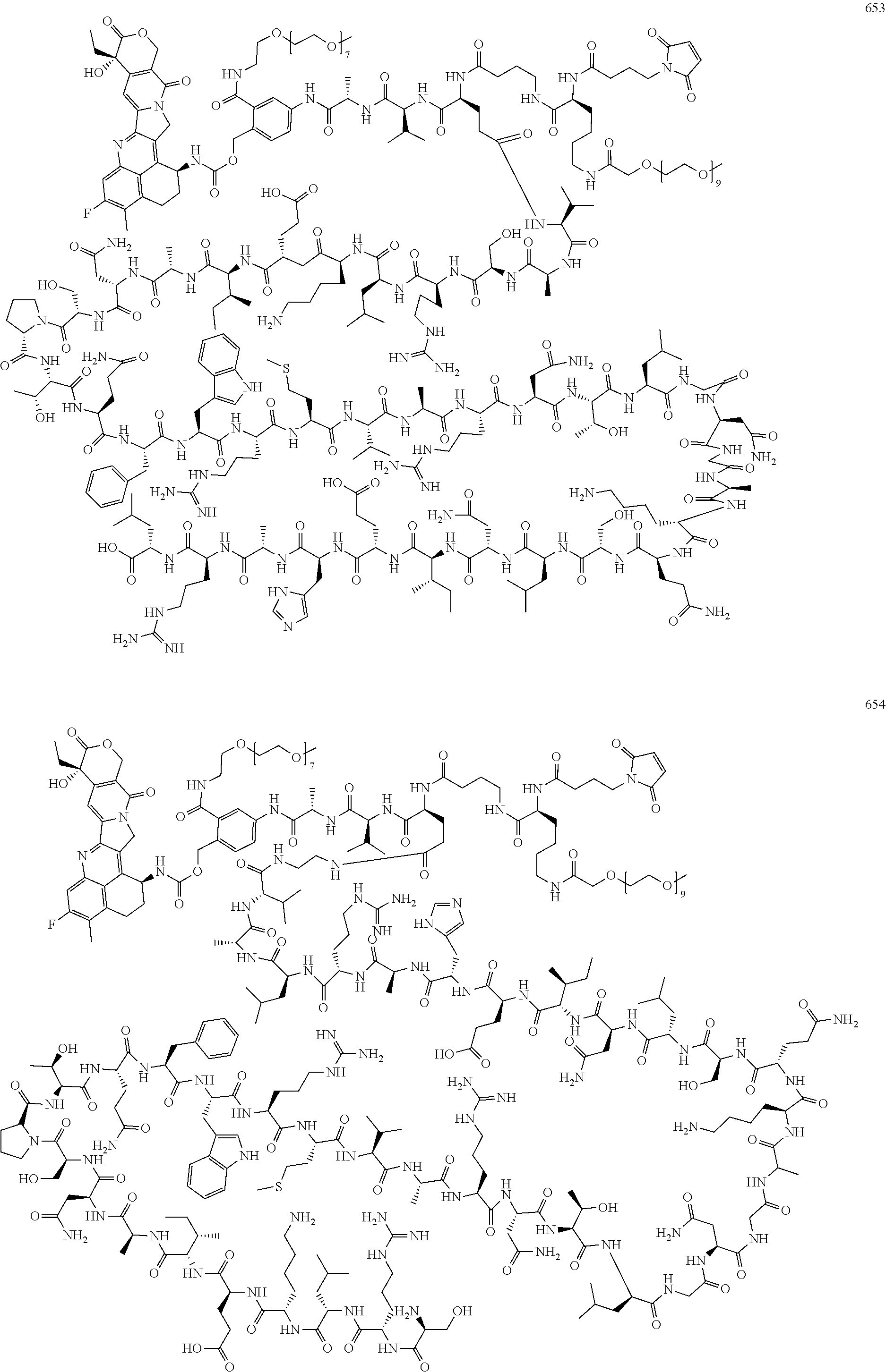

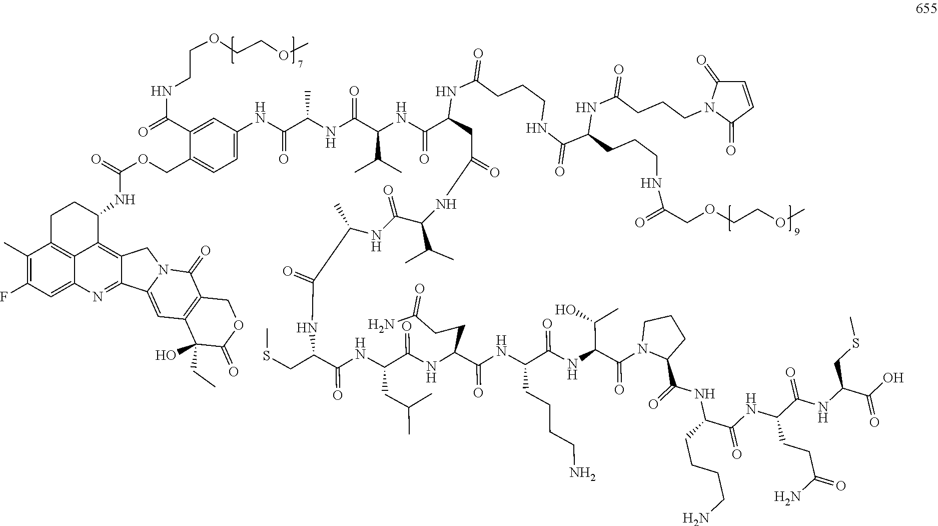

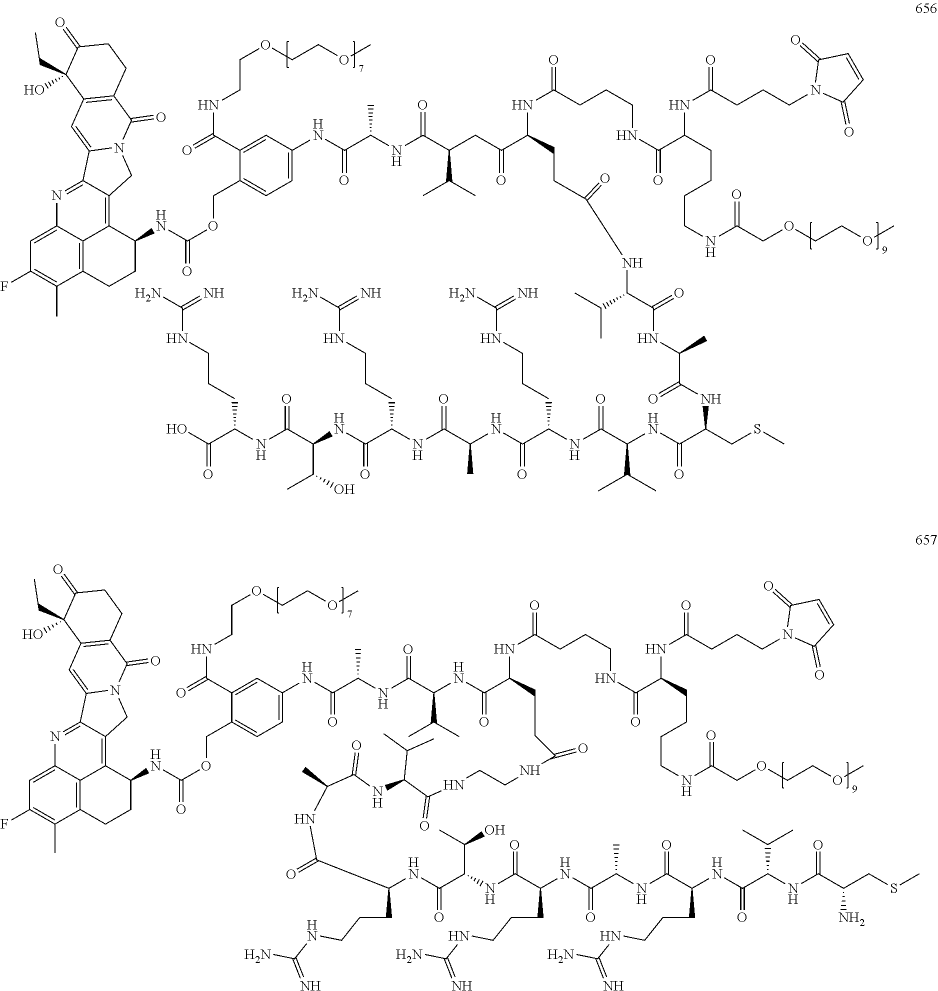

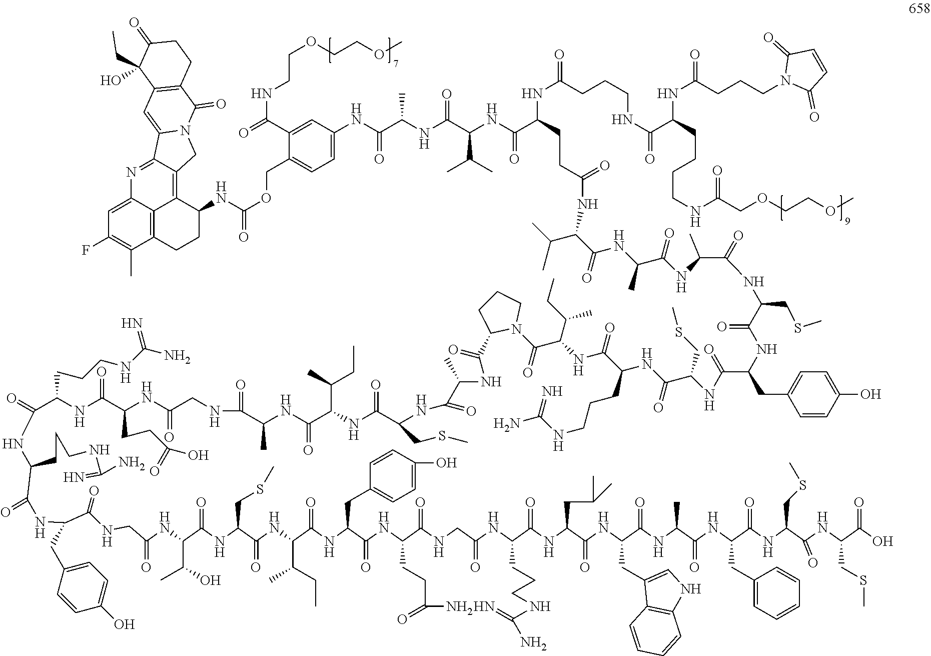

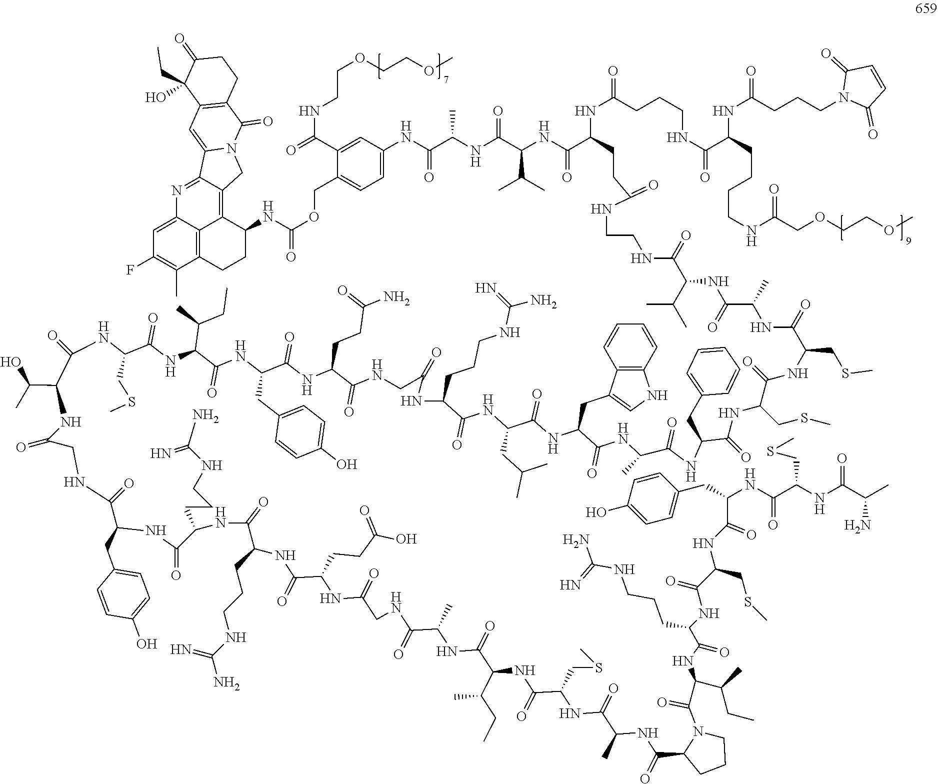

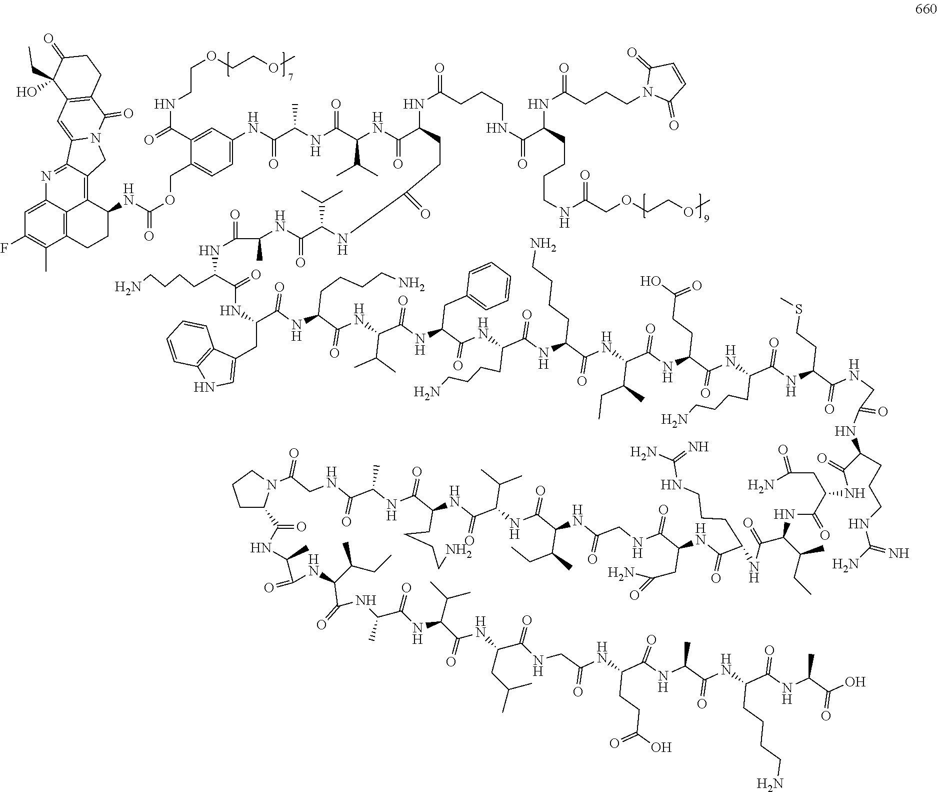

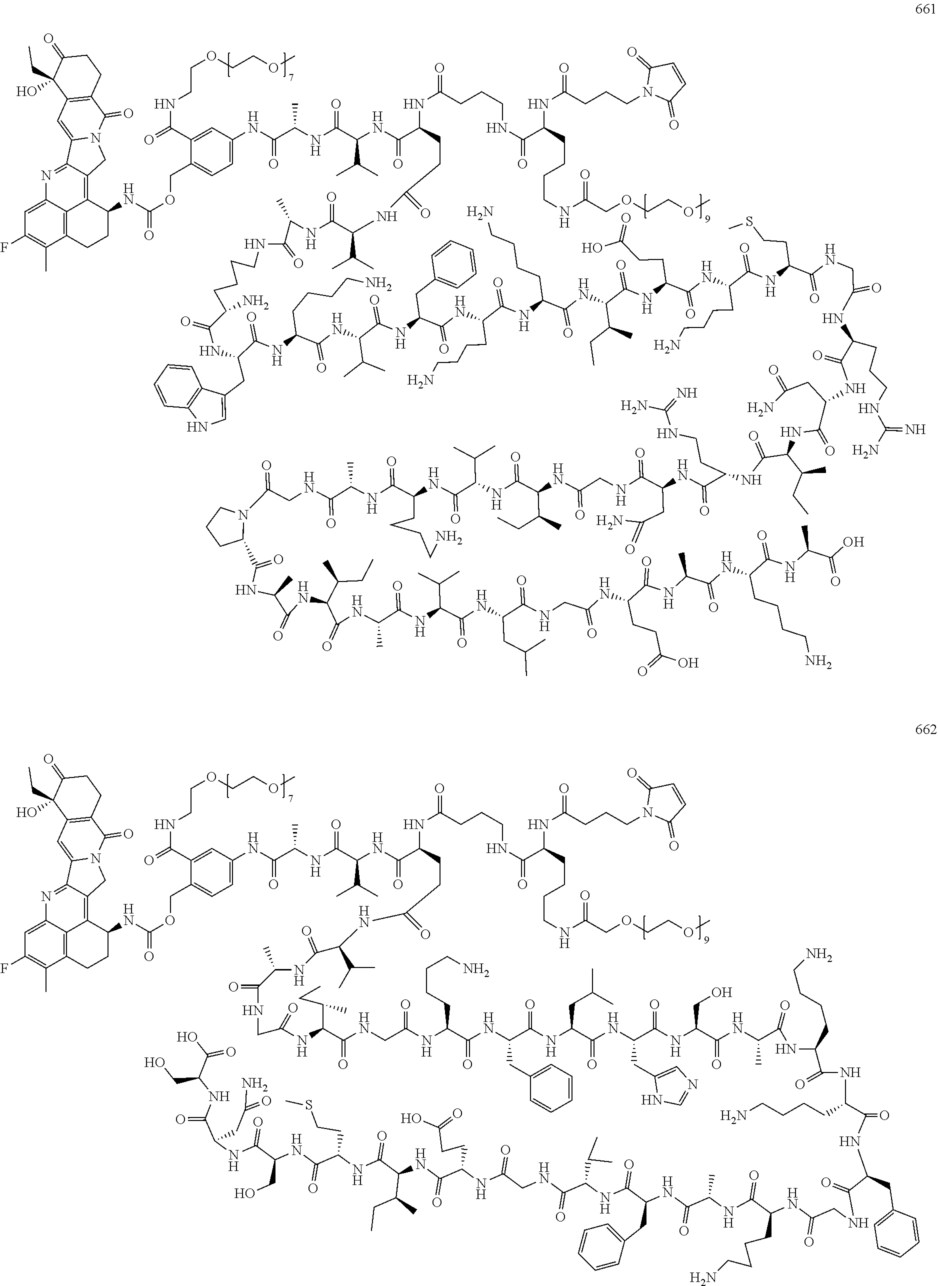

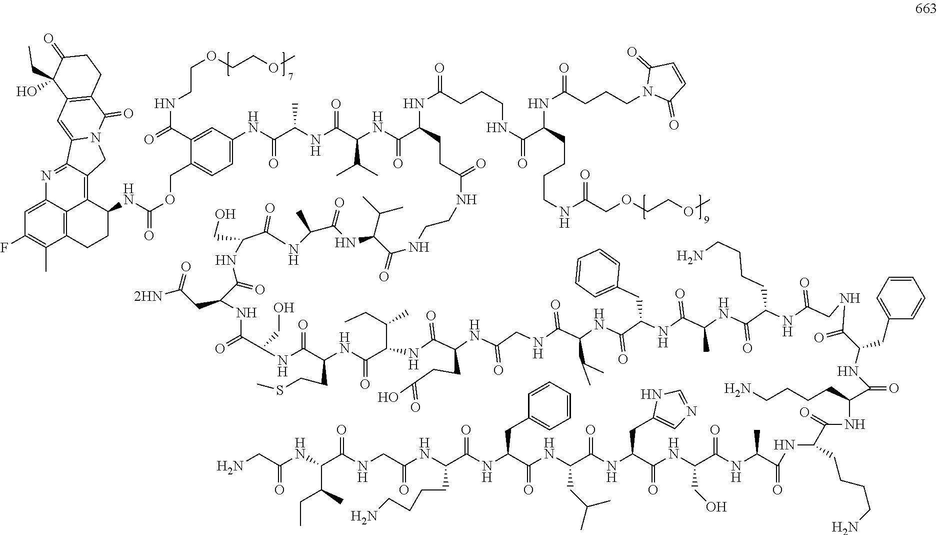

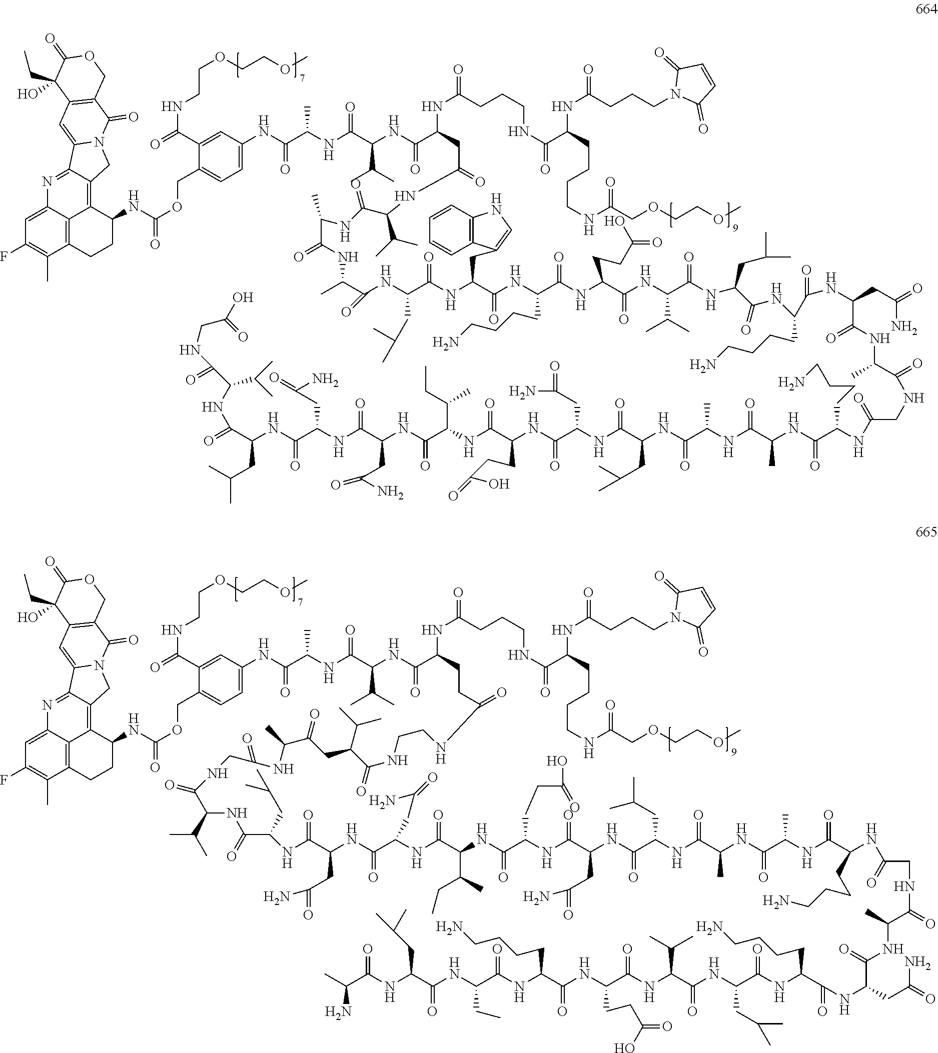

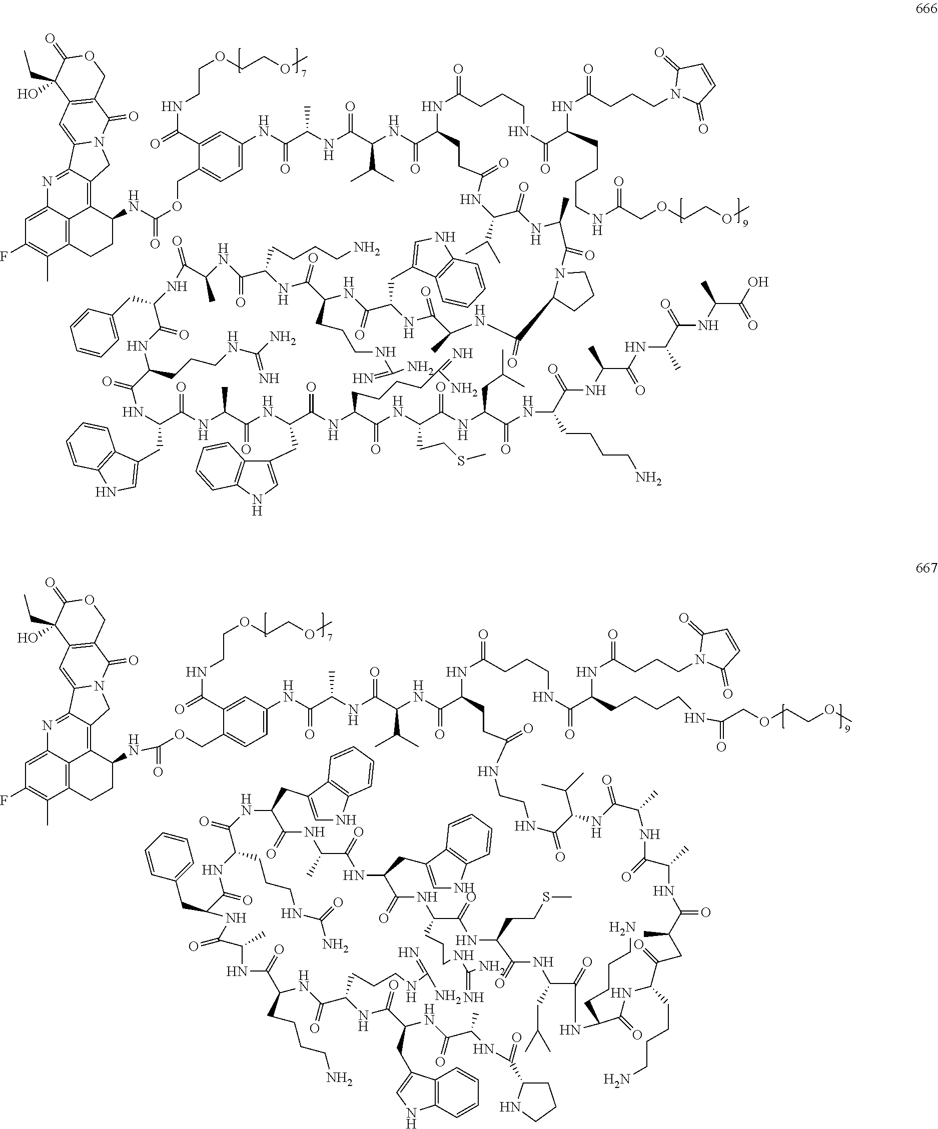

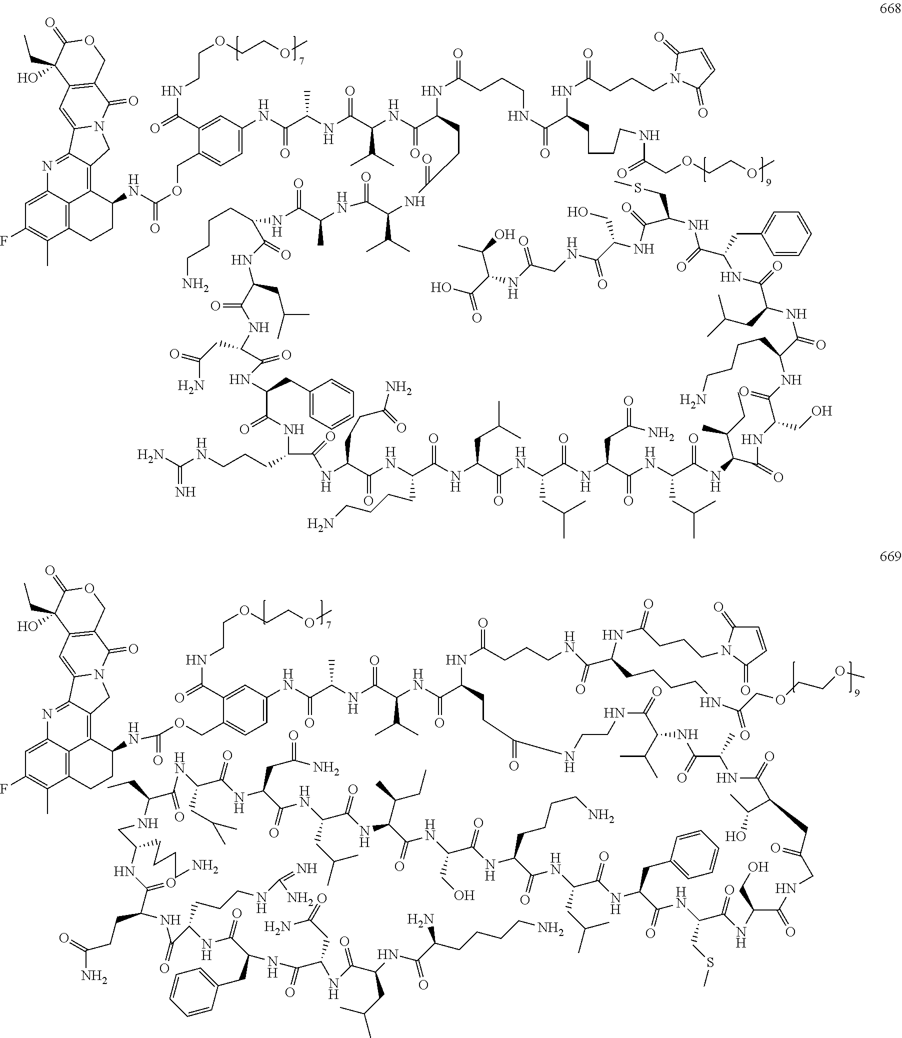

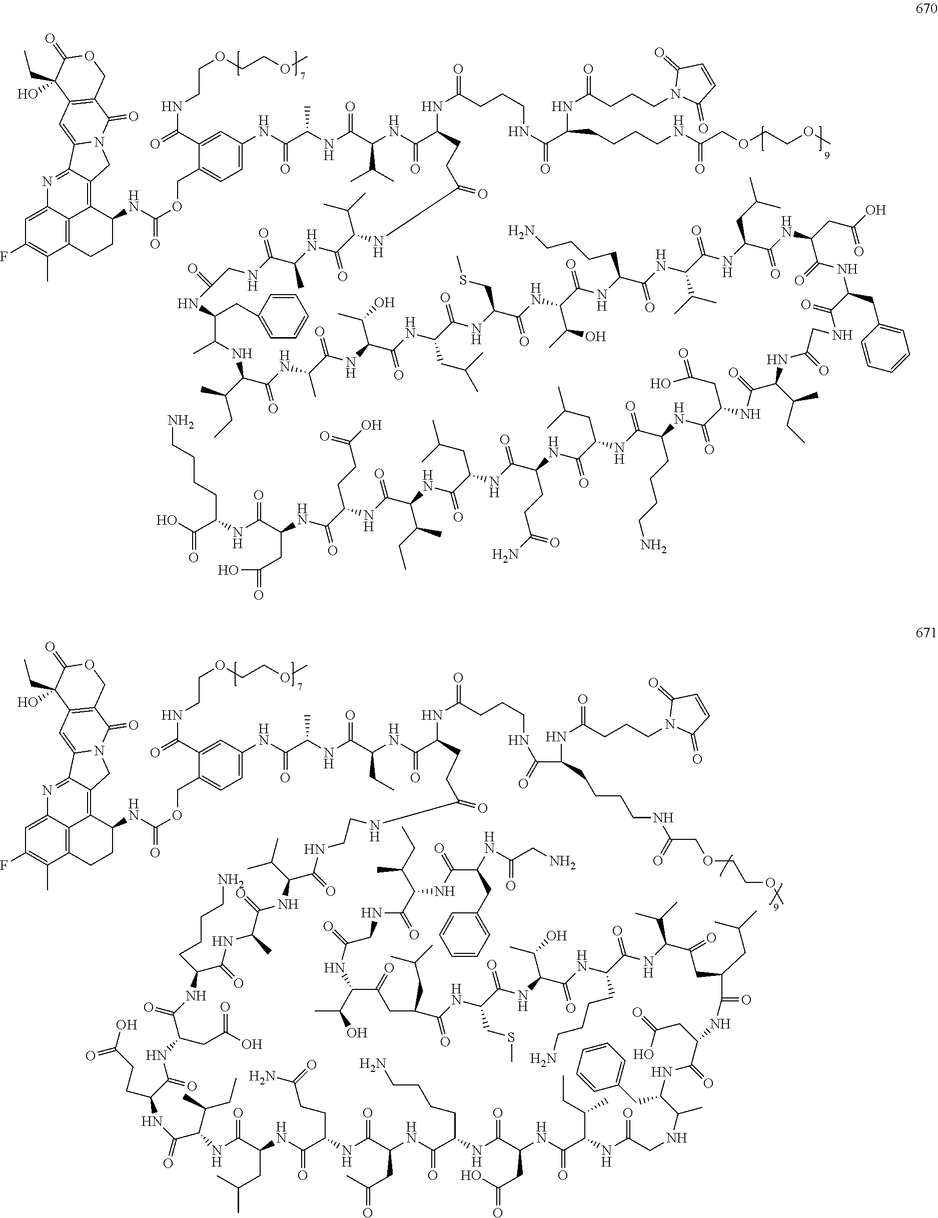

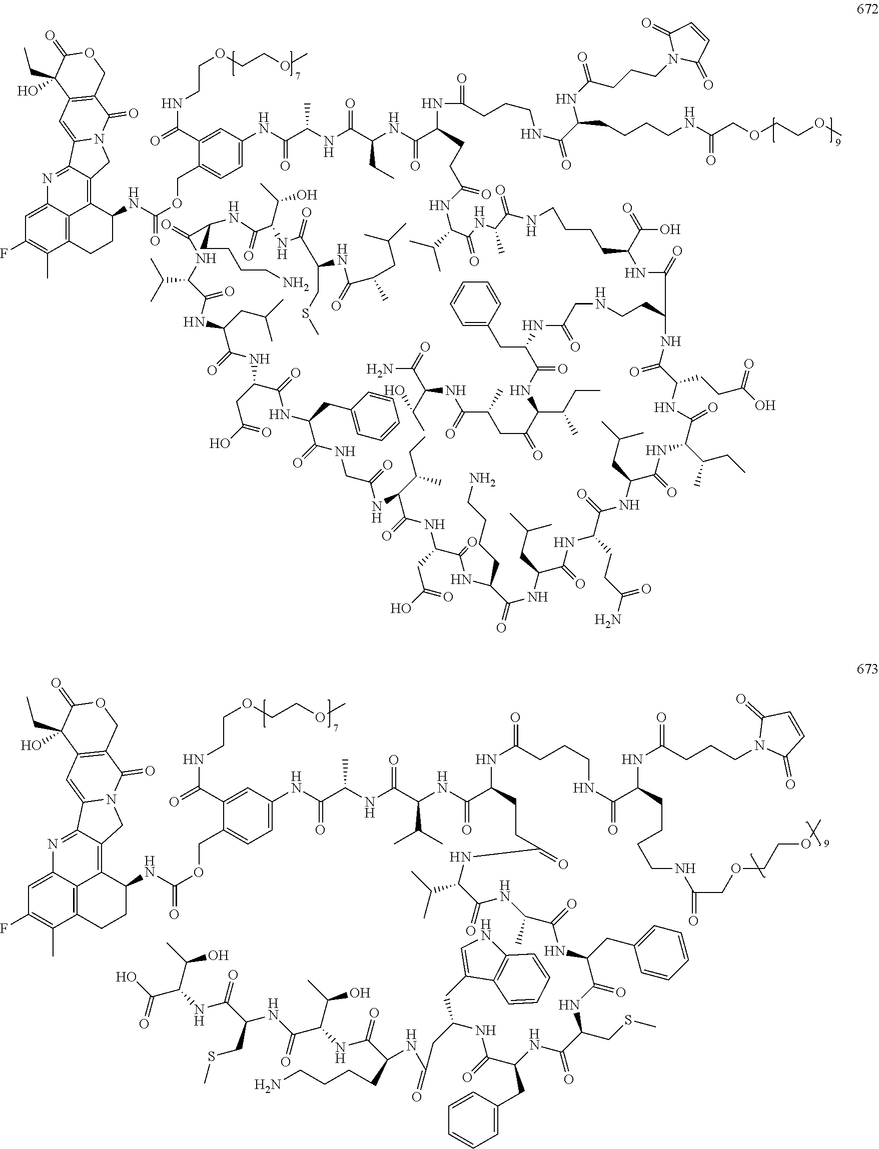

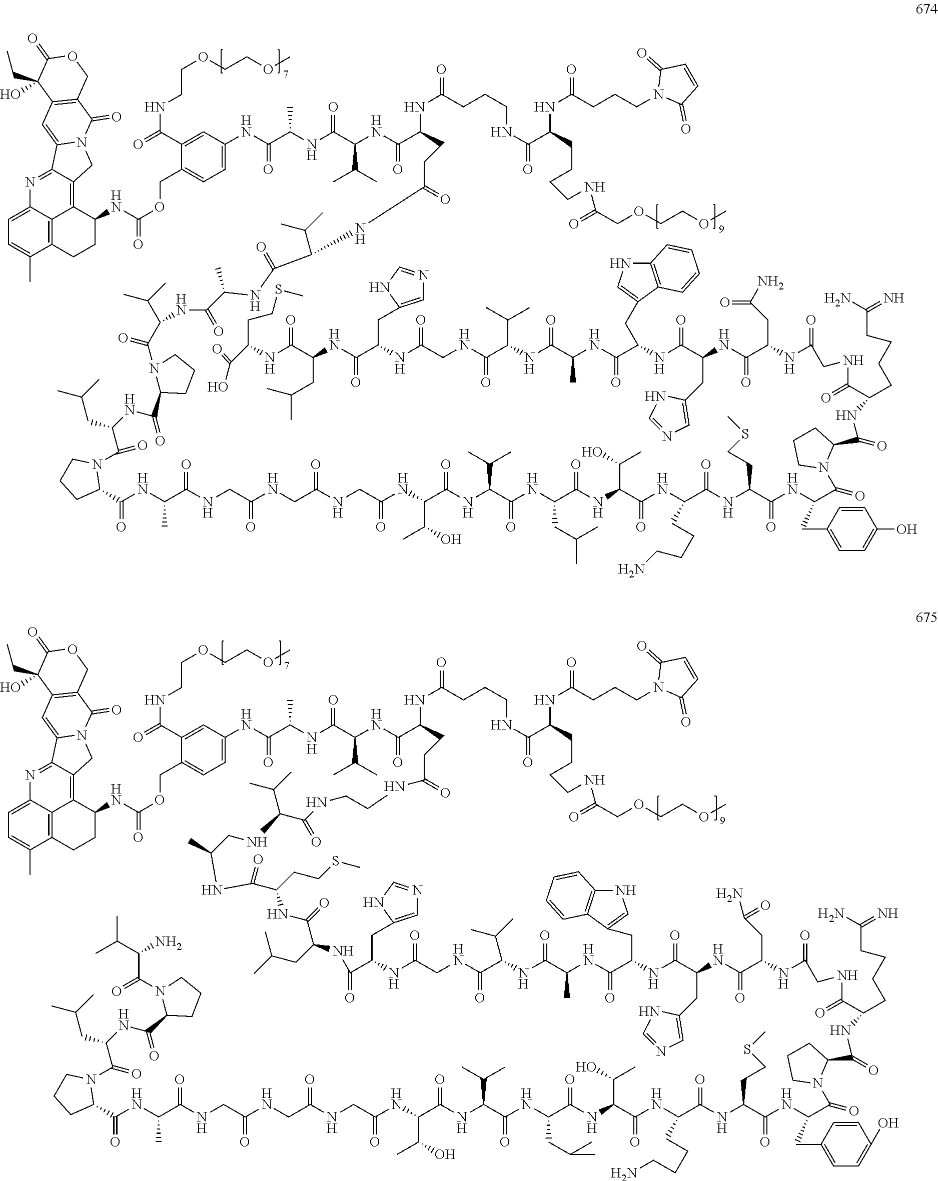

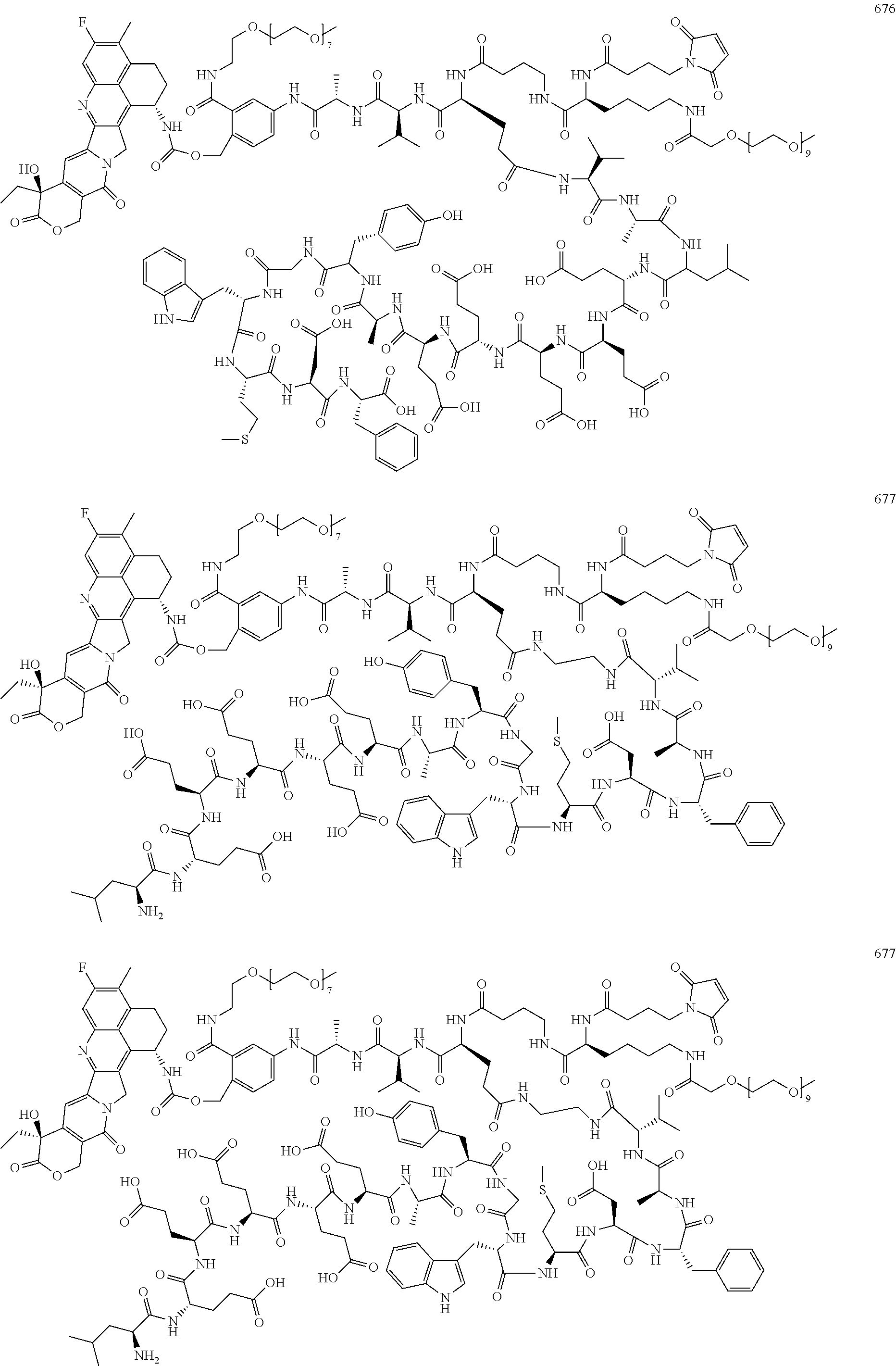

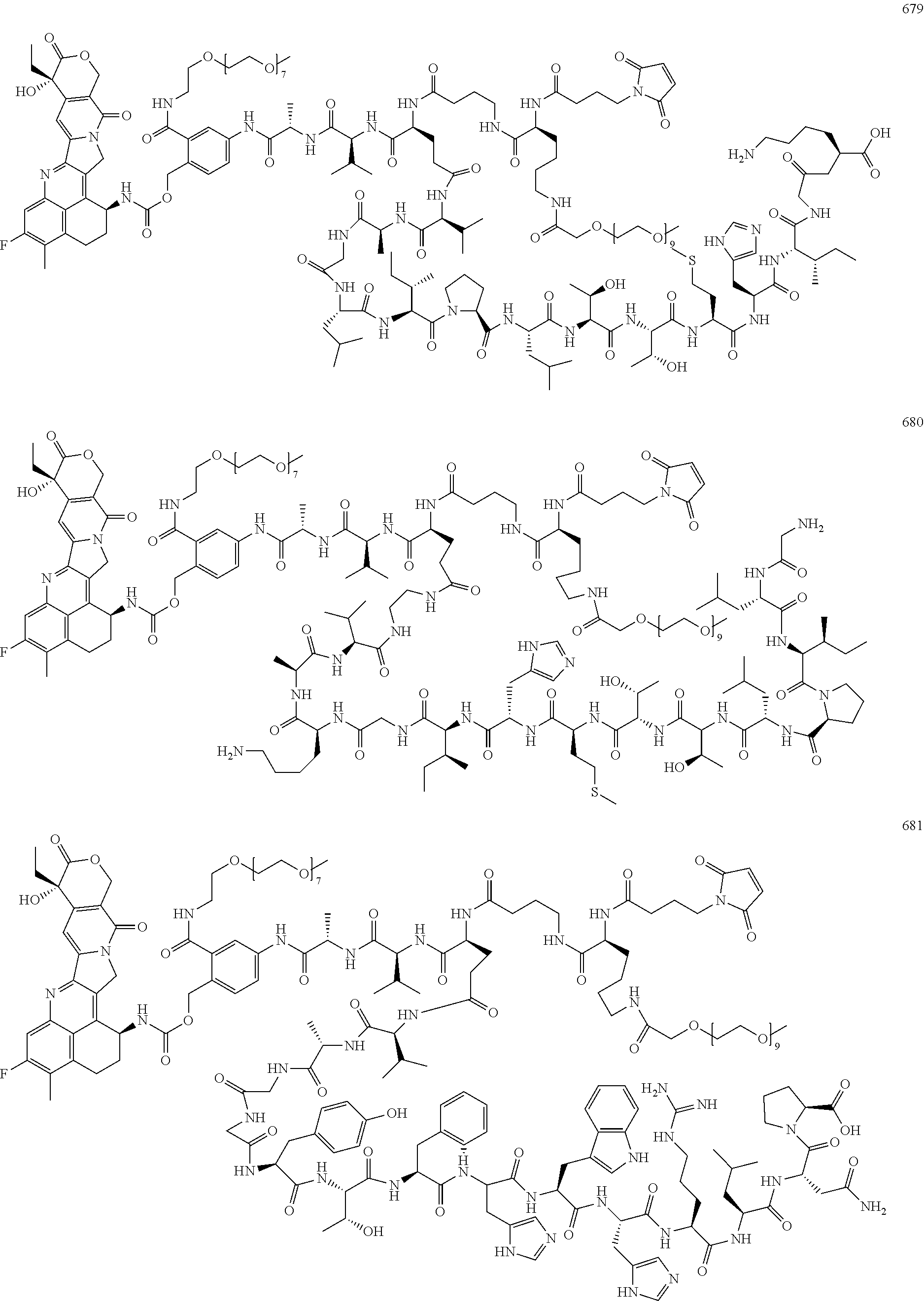

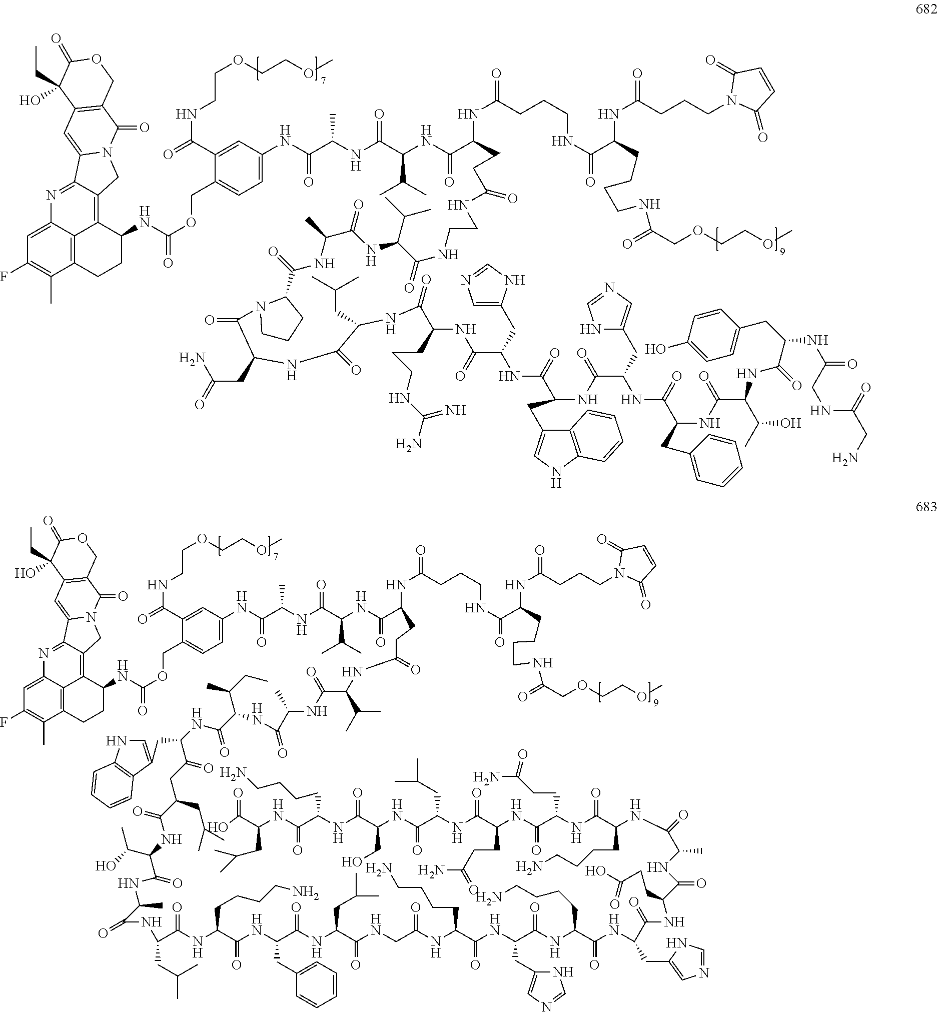

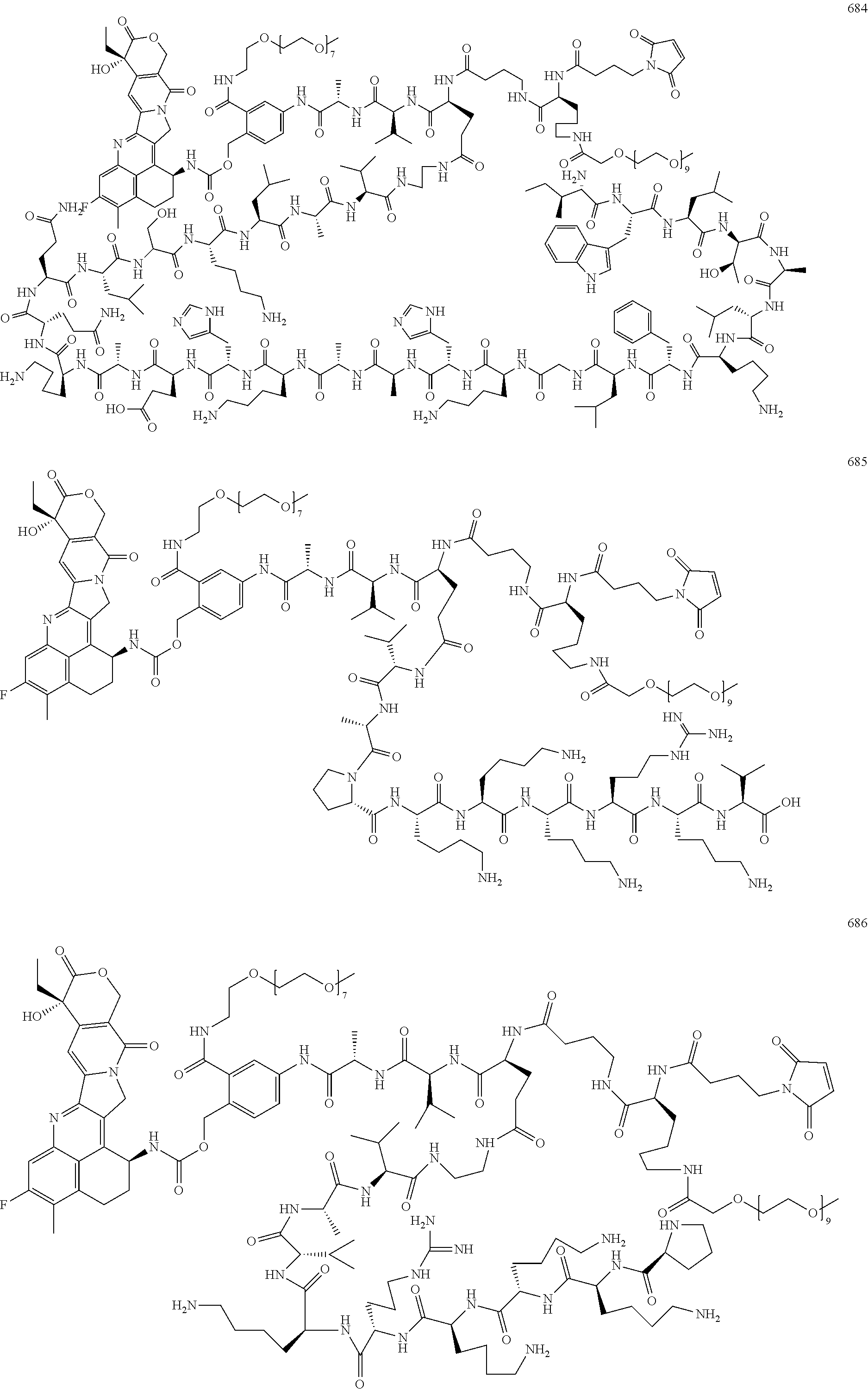

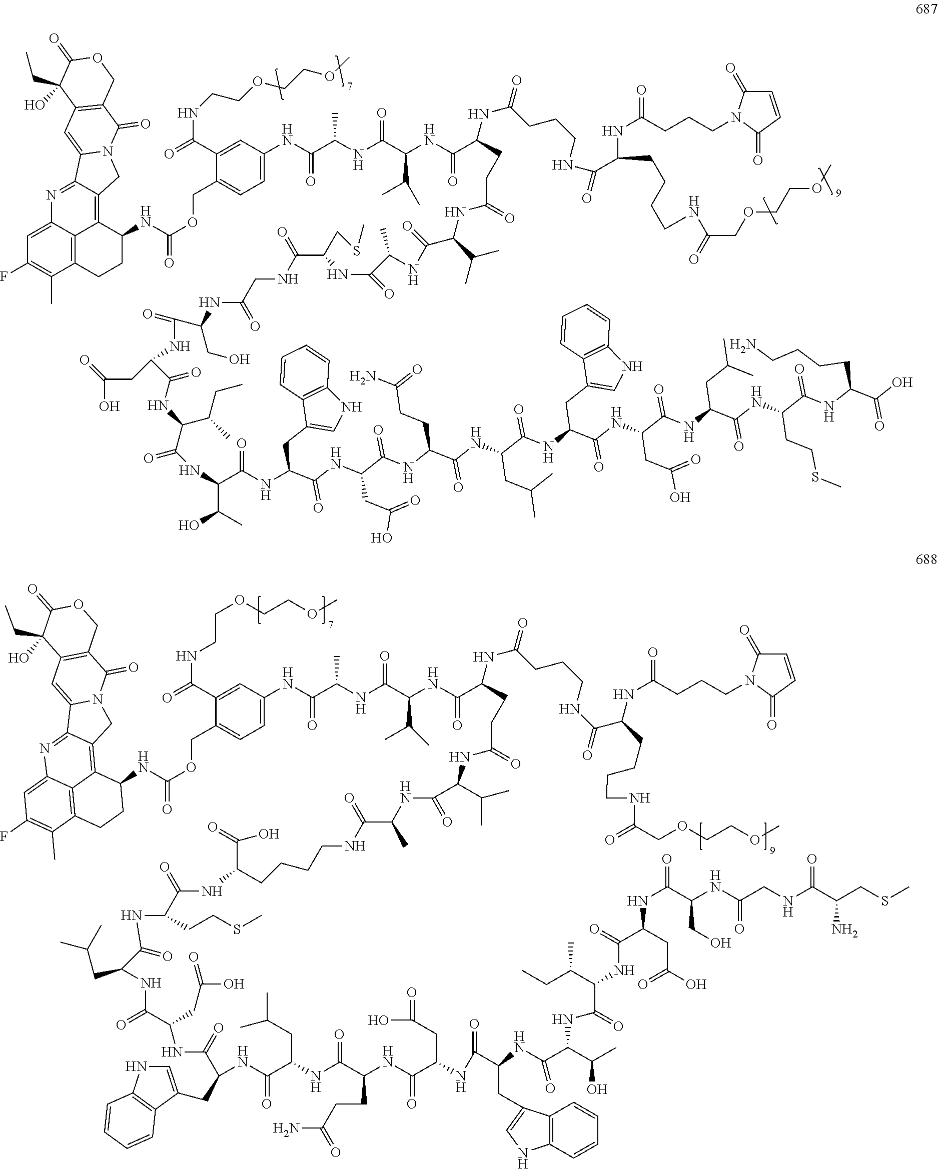

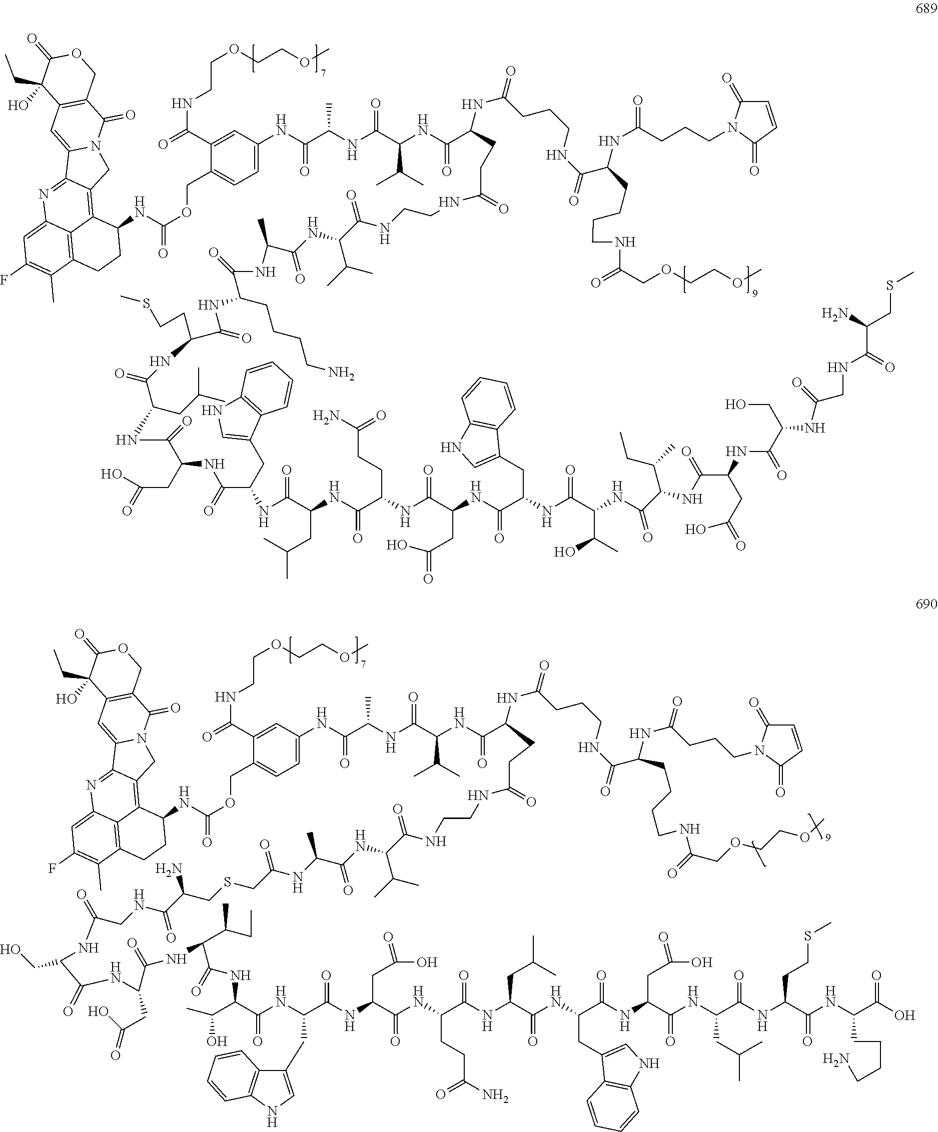

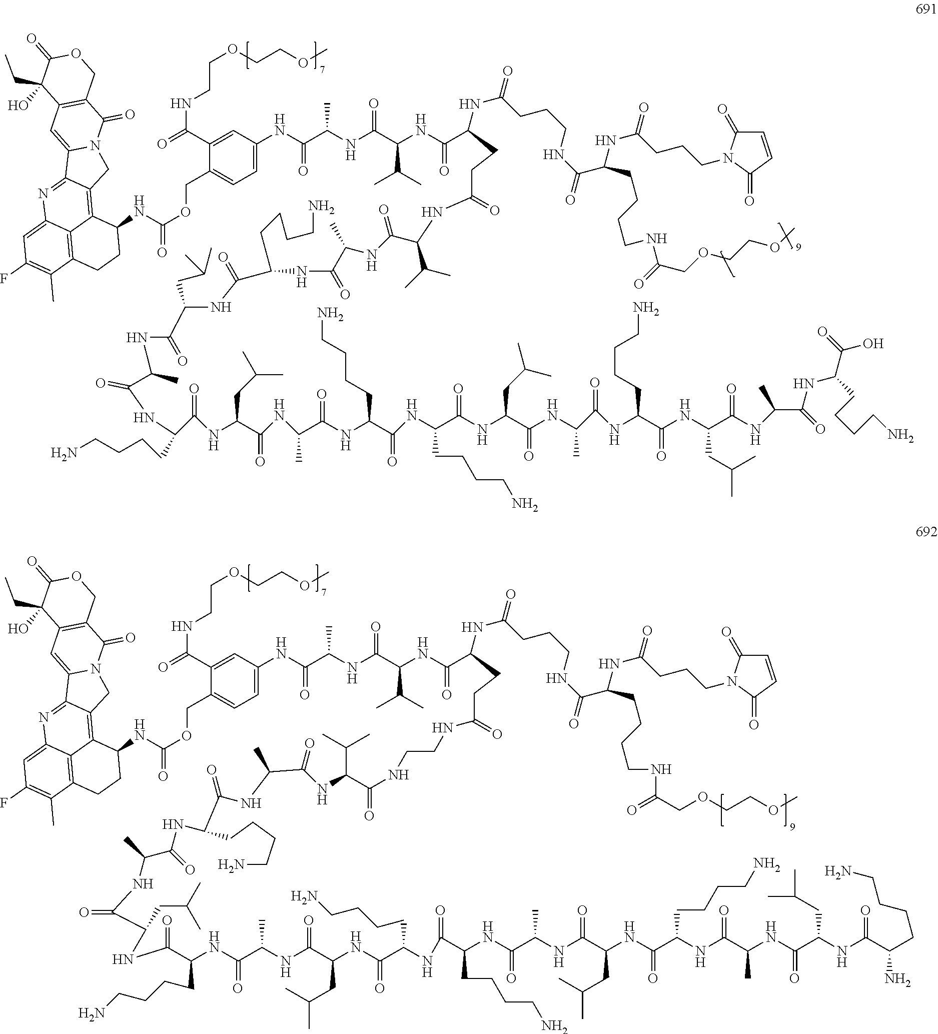

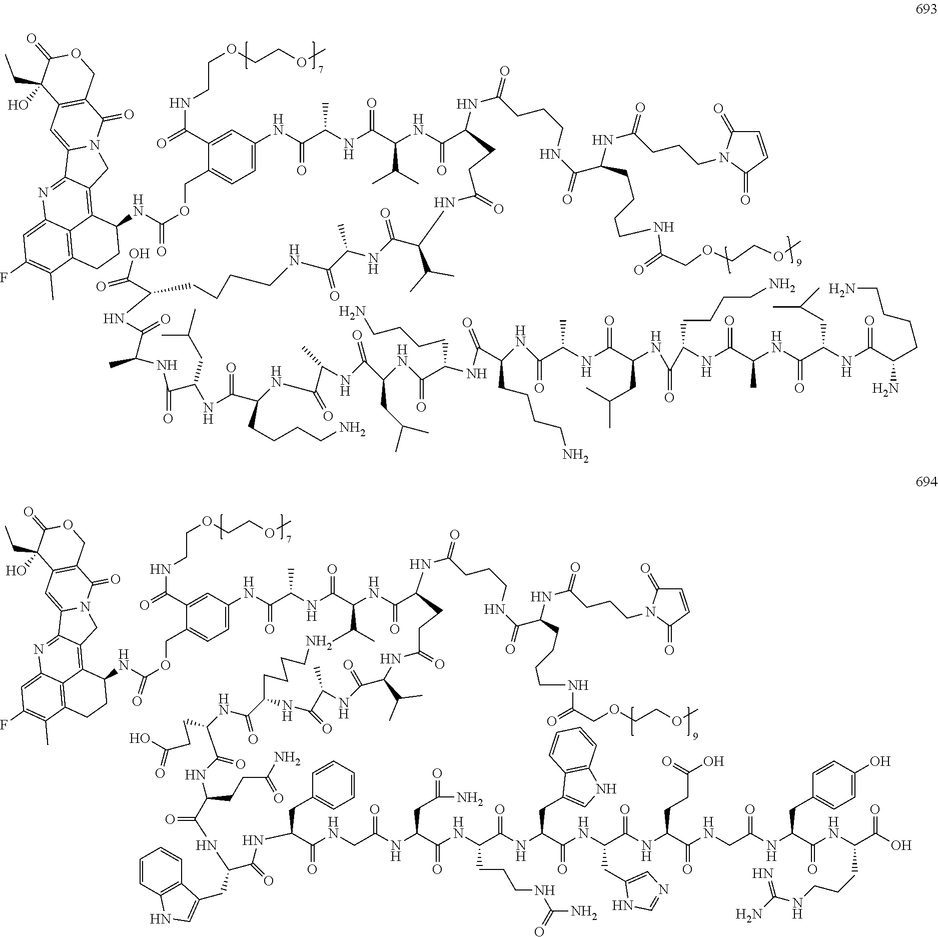

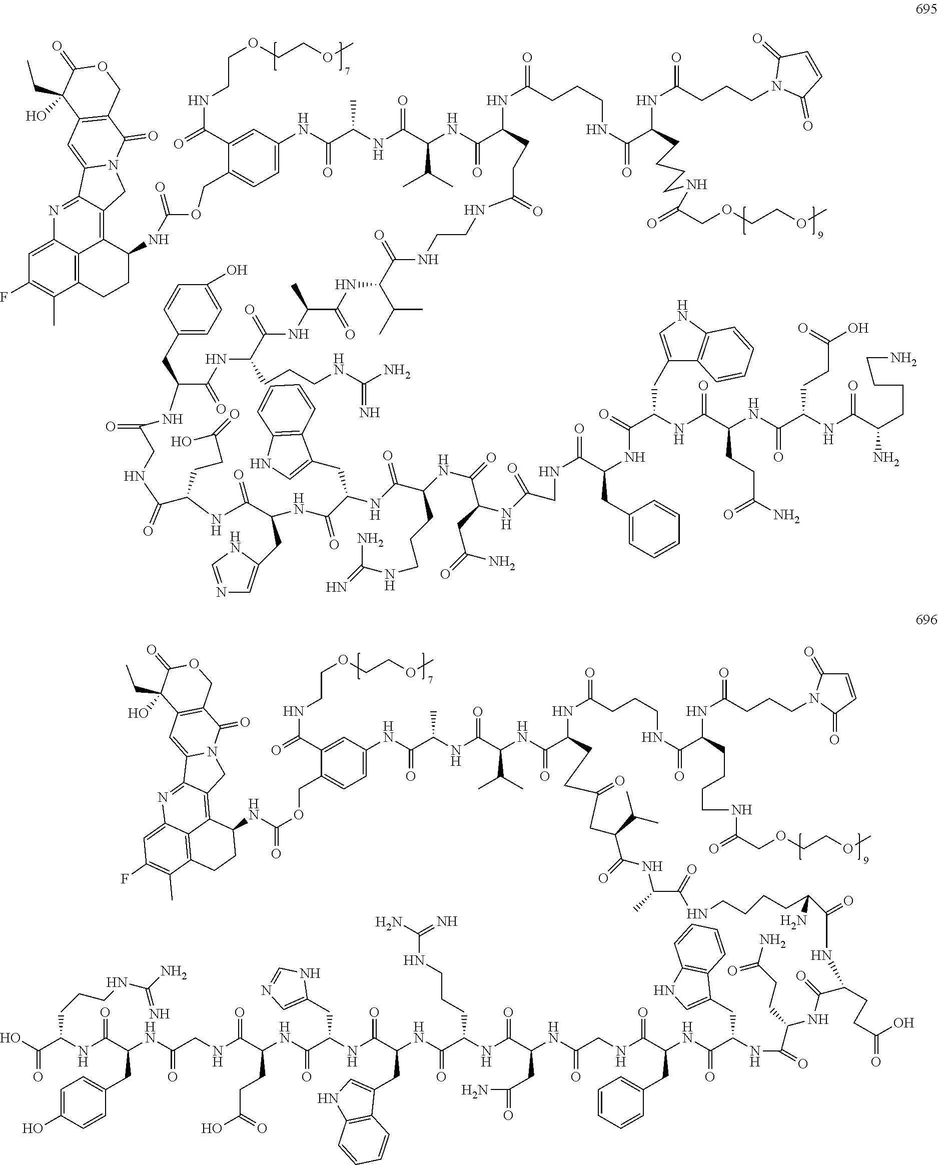

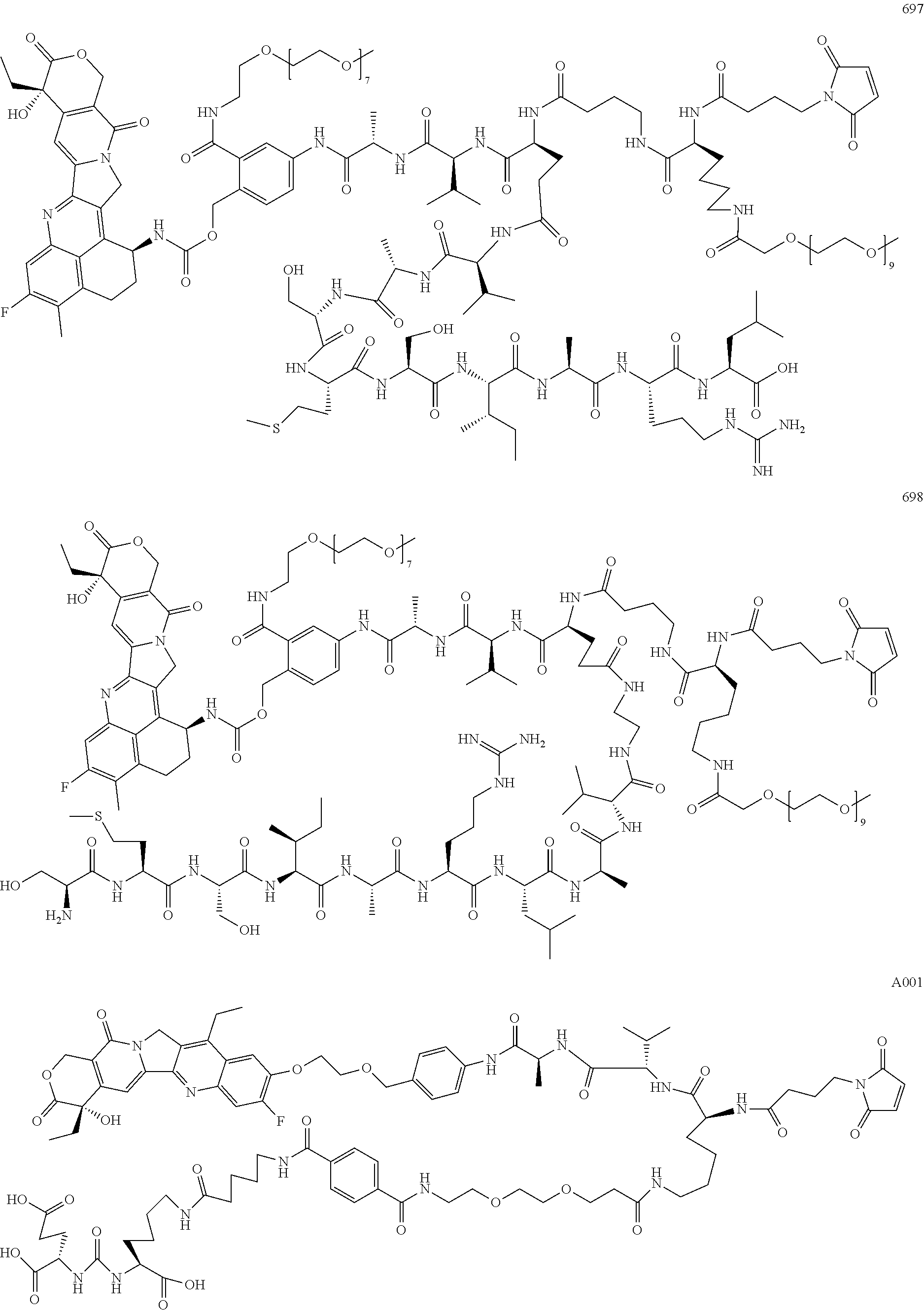

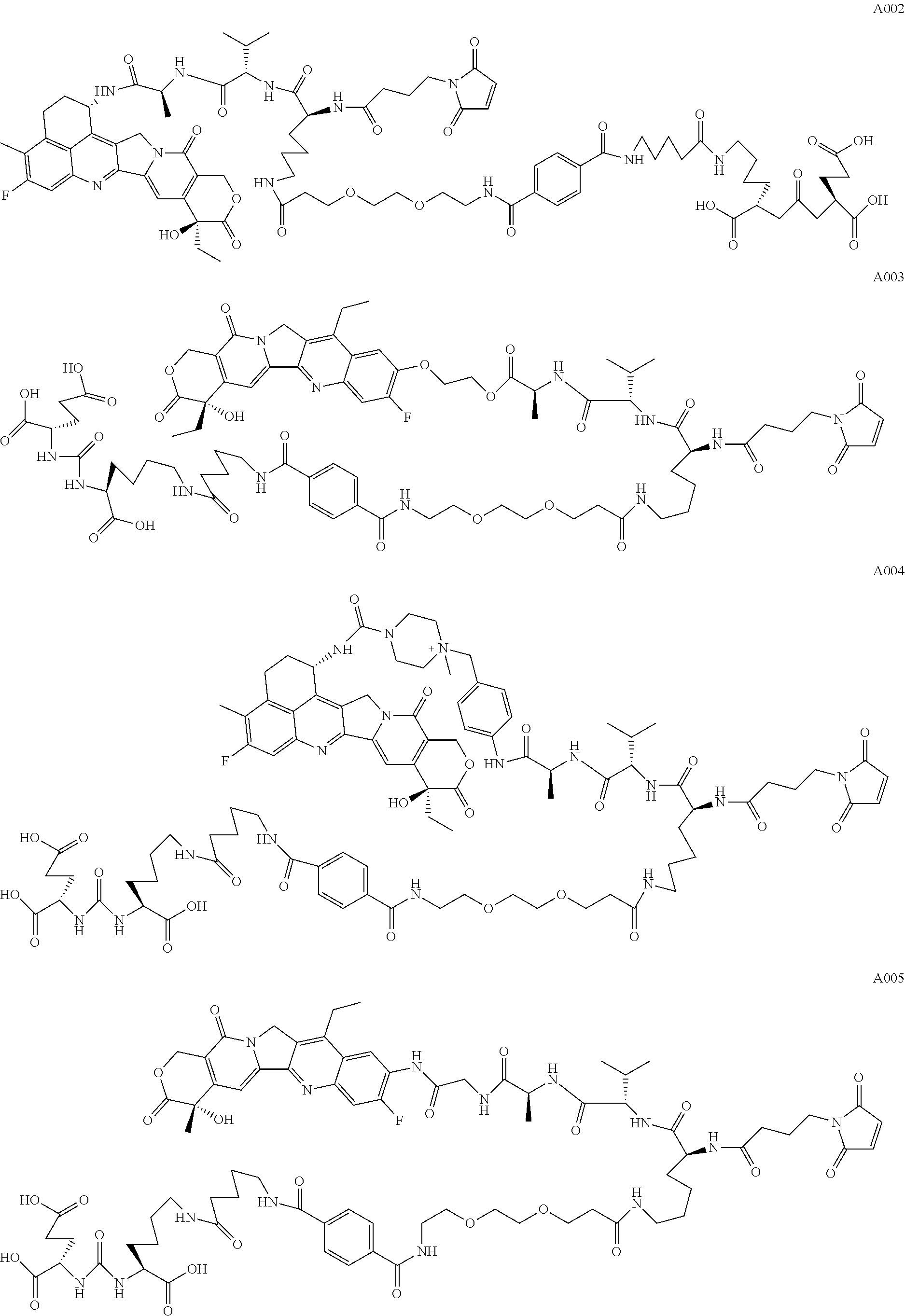

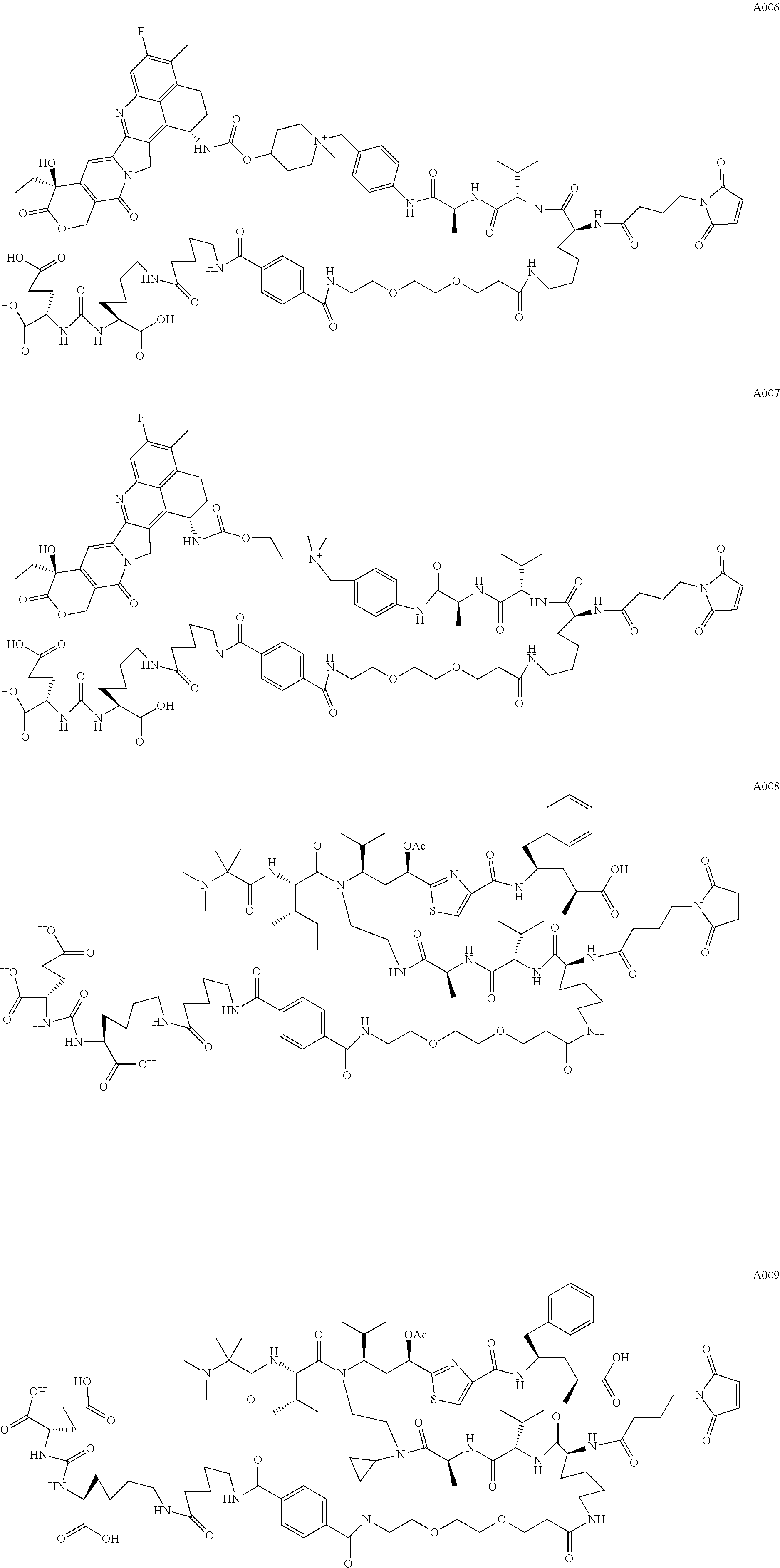

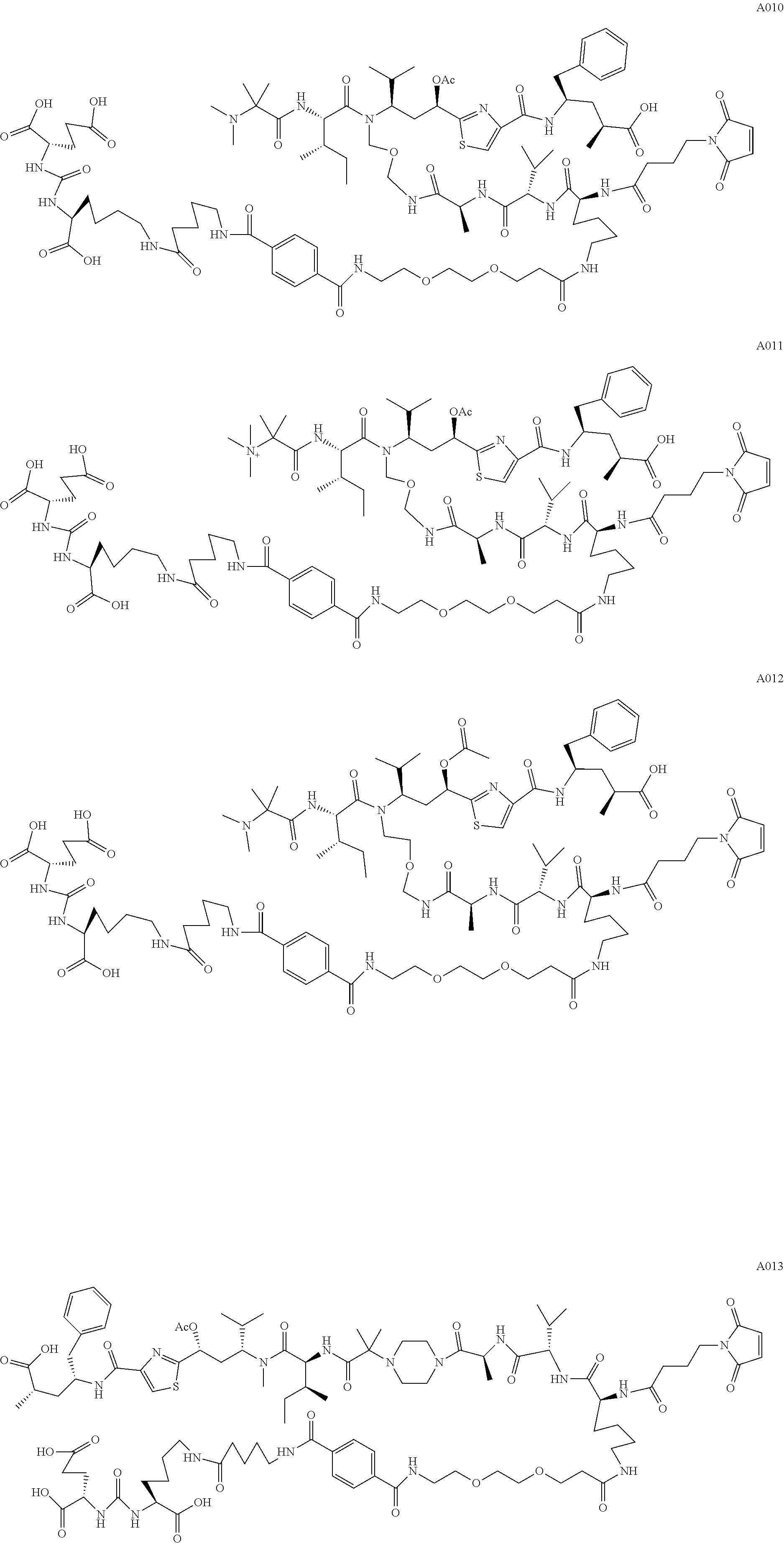

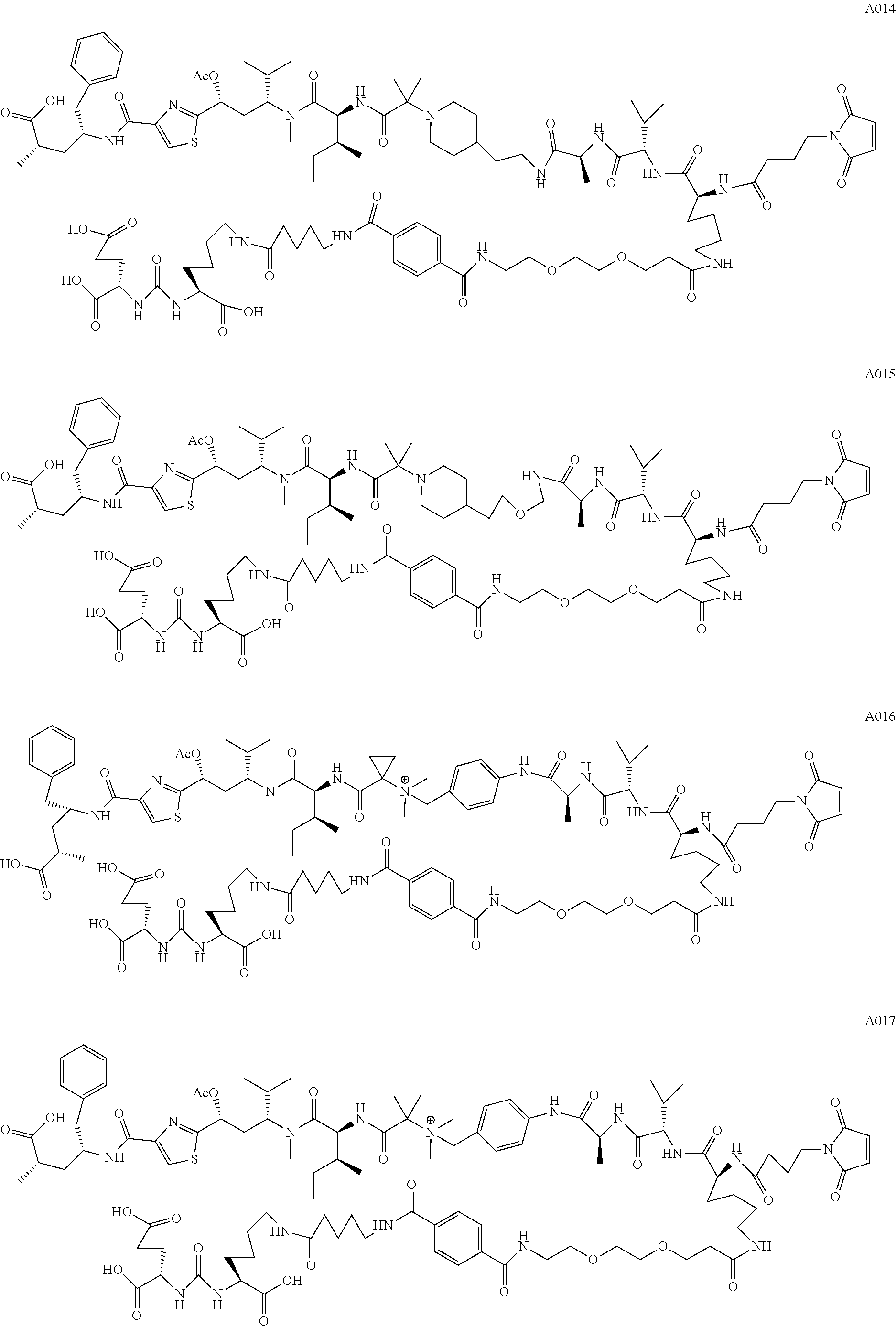

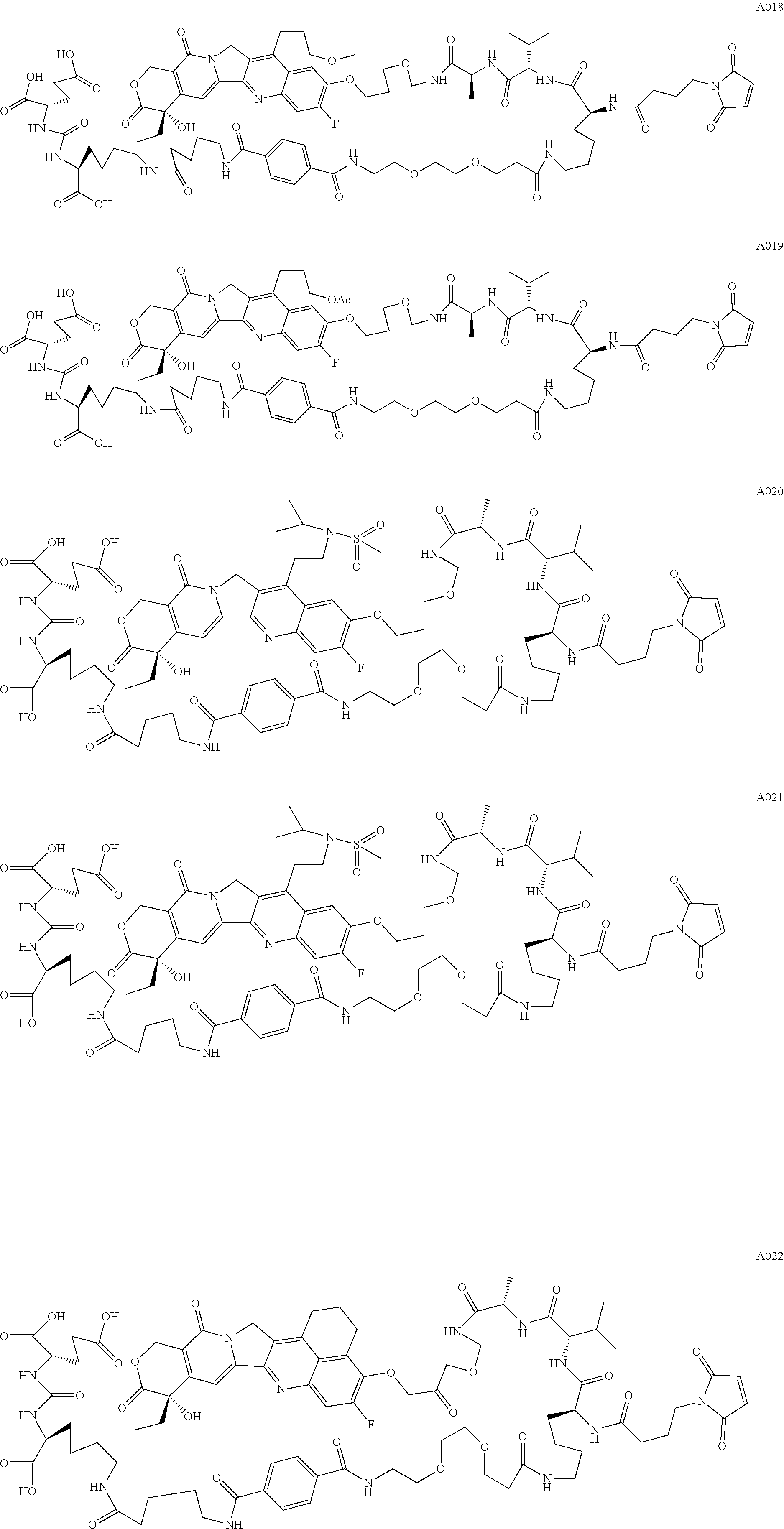

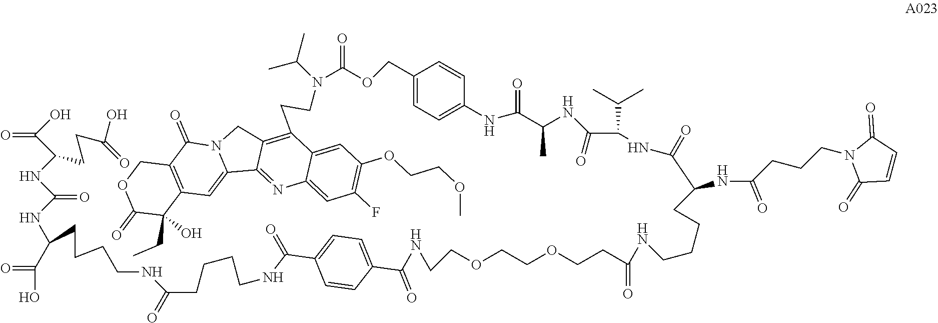 [SMILES: CC[C@@]1(O)C(=O)OCc2c1cc1n(c2=O)Cc2c-1nc1cc(F)c(OCCOC)cc1c2CCN(C(=O)OCc1ccc(NC(=O)[C@H](C)NC(=O)[C@@H](NC(=O)[C@H](CCCCNC(=O)CCOCCOCCNC(=O)c2ccc(C(=O)NCCCCC(=O)NCCCC[C@H](NC(=O)N[C@@H](CCC(=O)O)C(=O)O)C(=O)O)cc2)NC(=O)CCCN2C(=O)C=CC2=O)C(C)C)cc1)C(C)C]

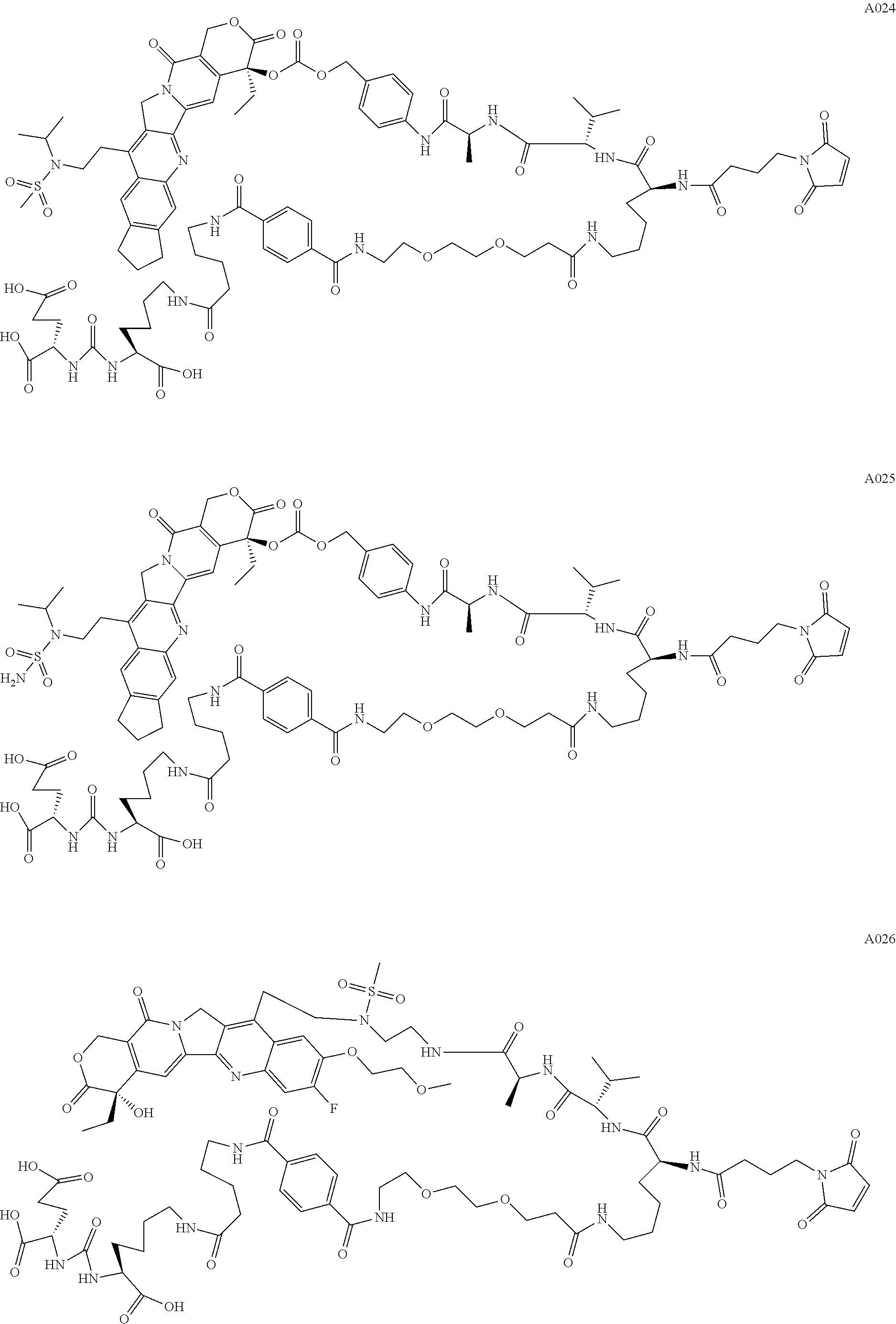

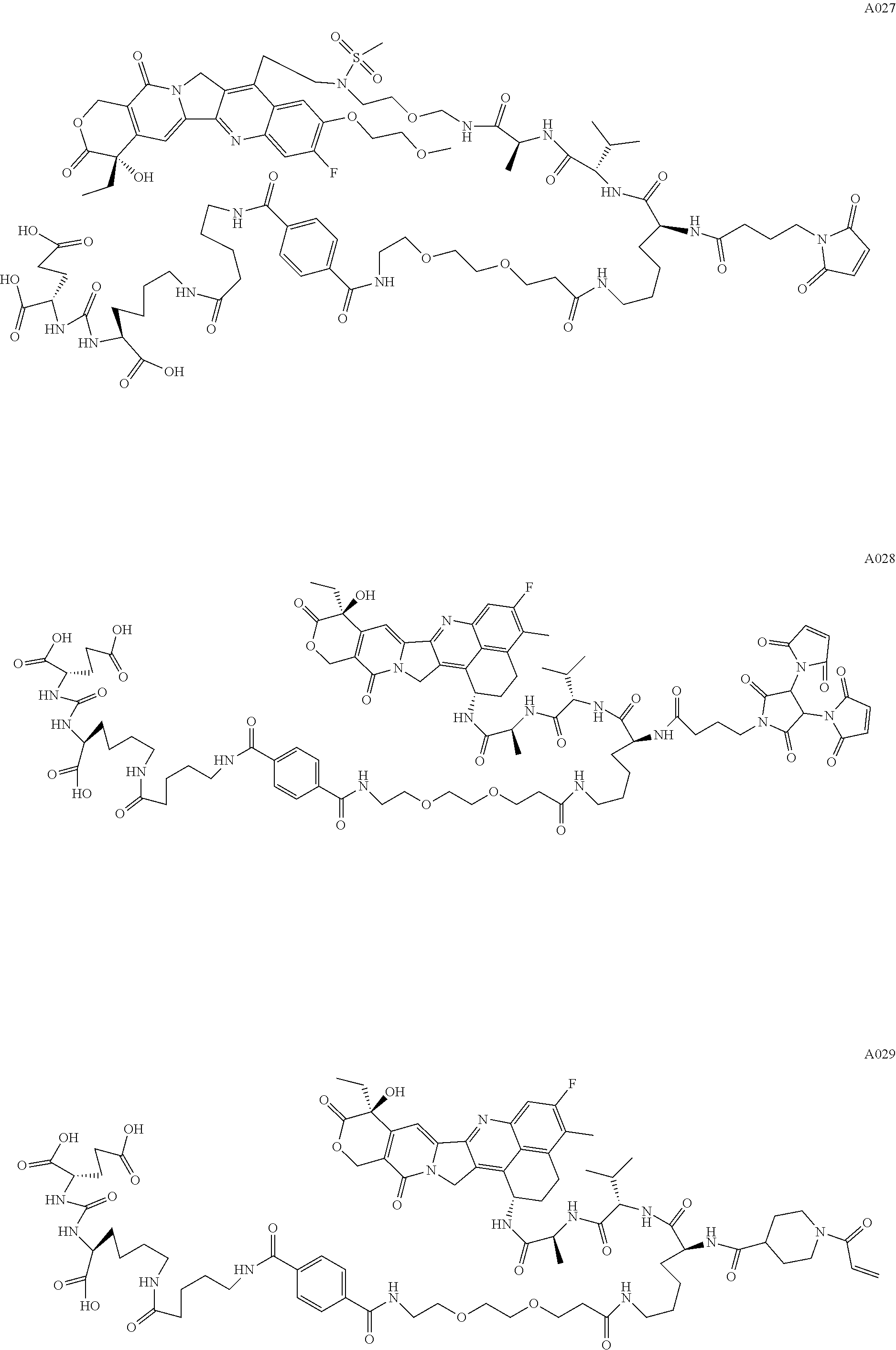

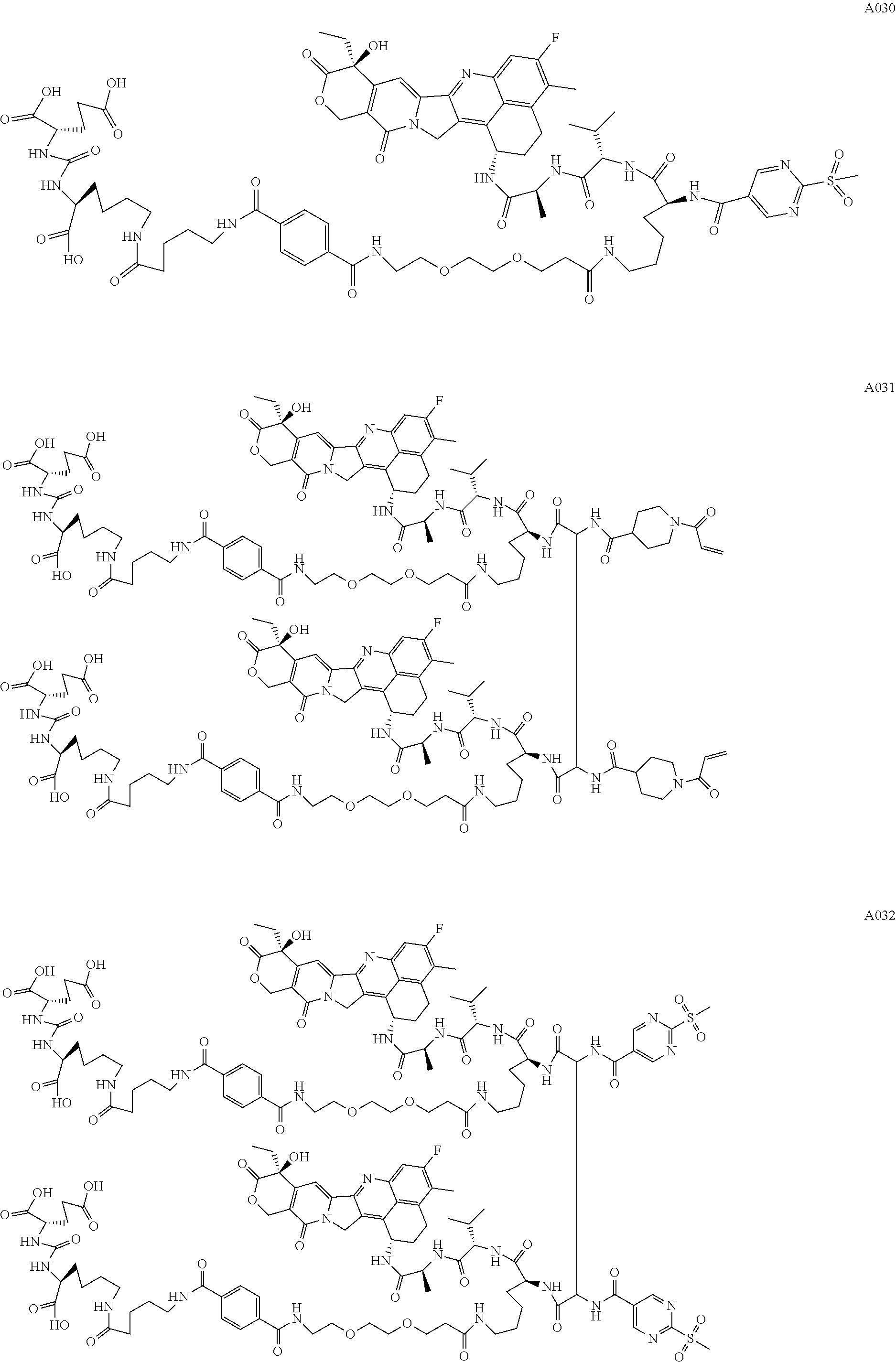

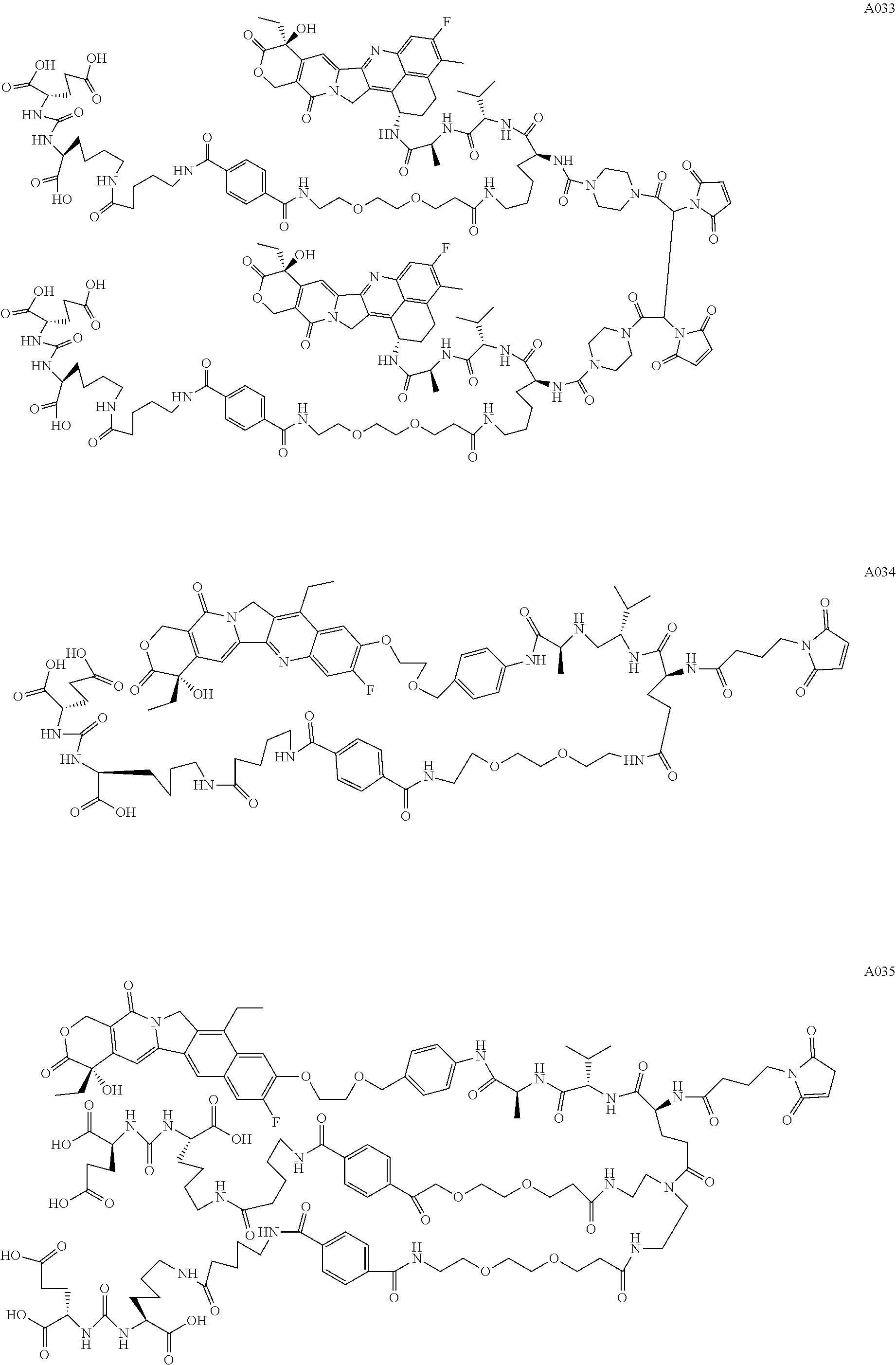

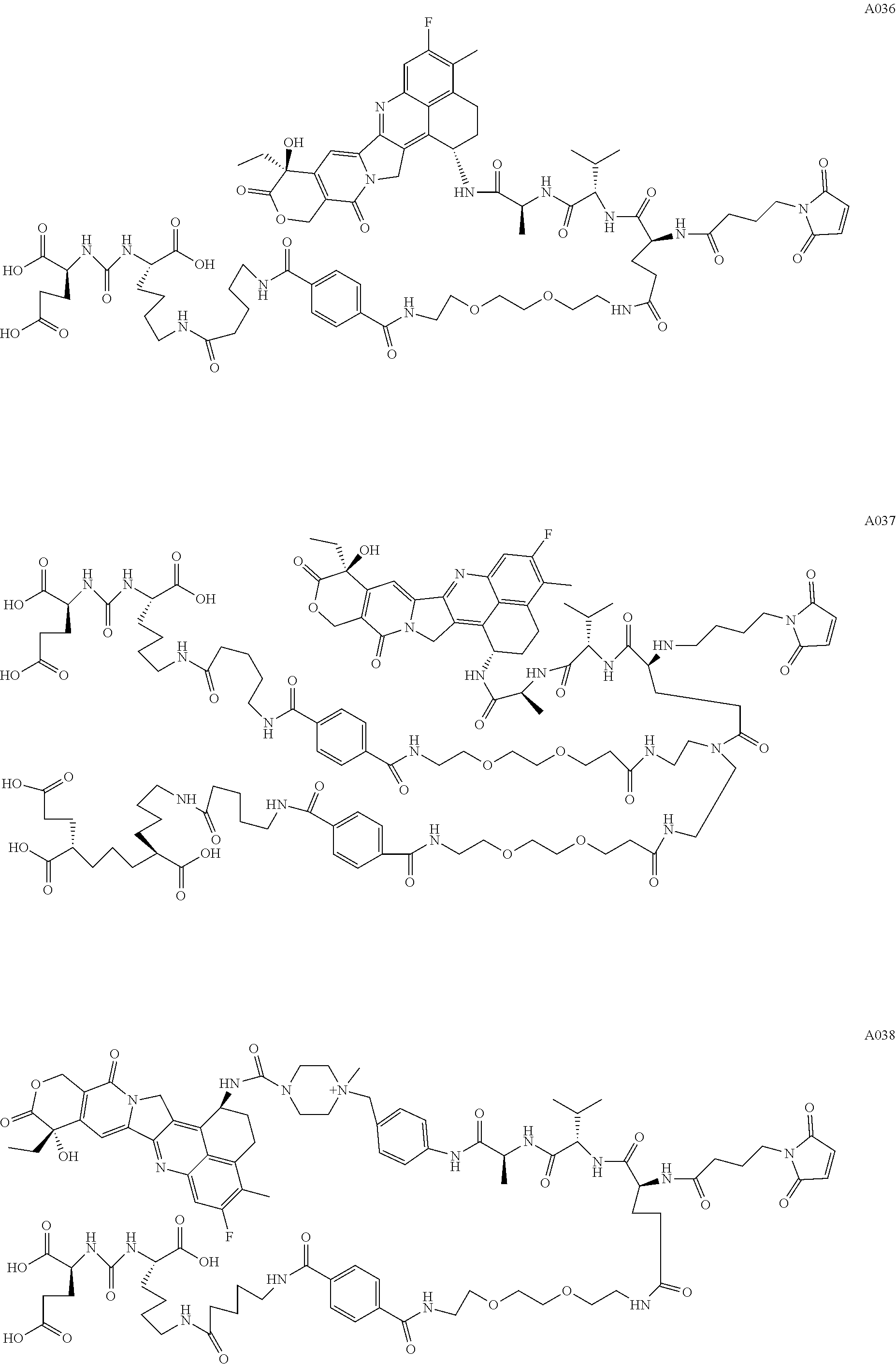

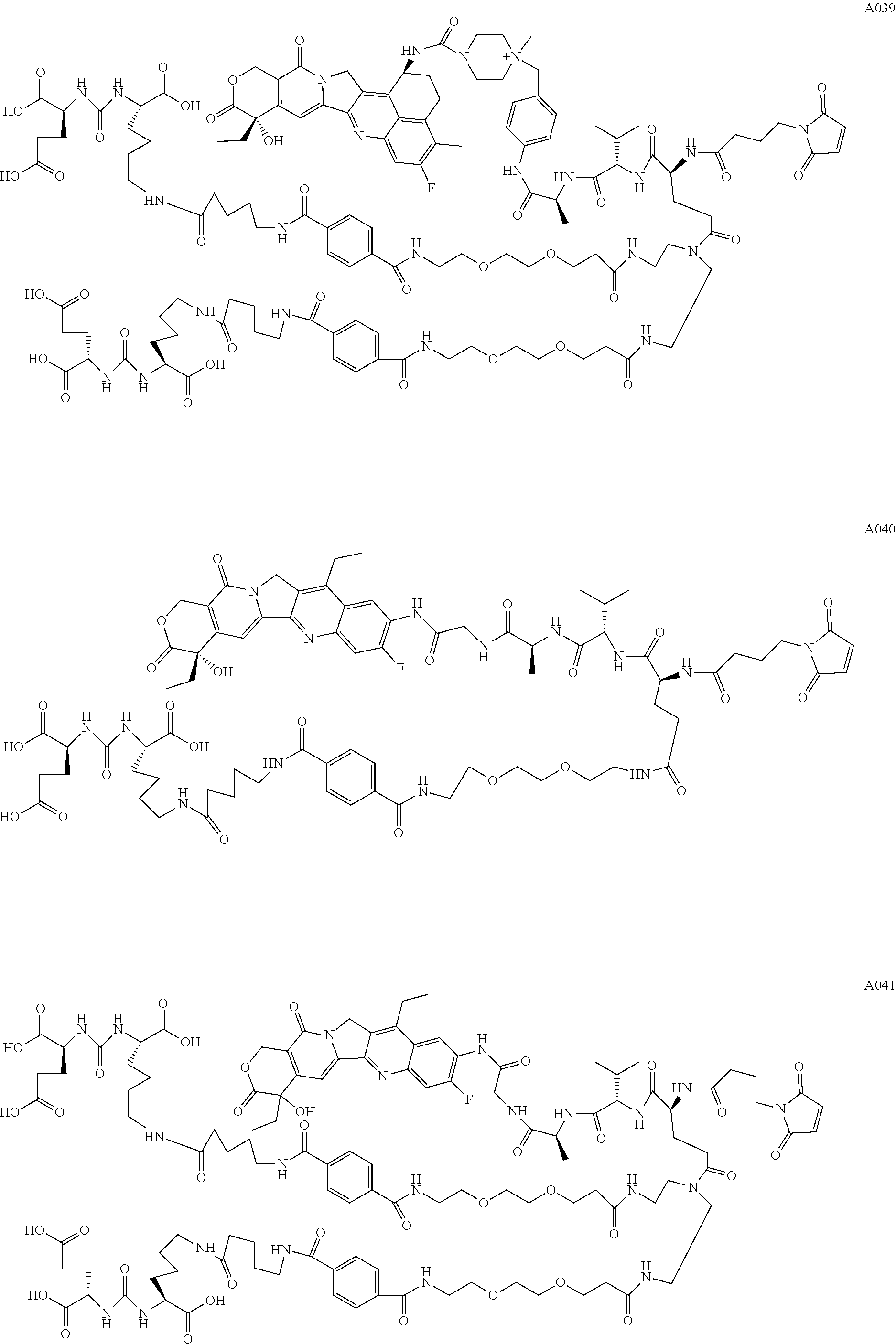

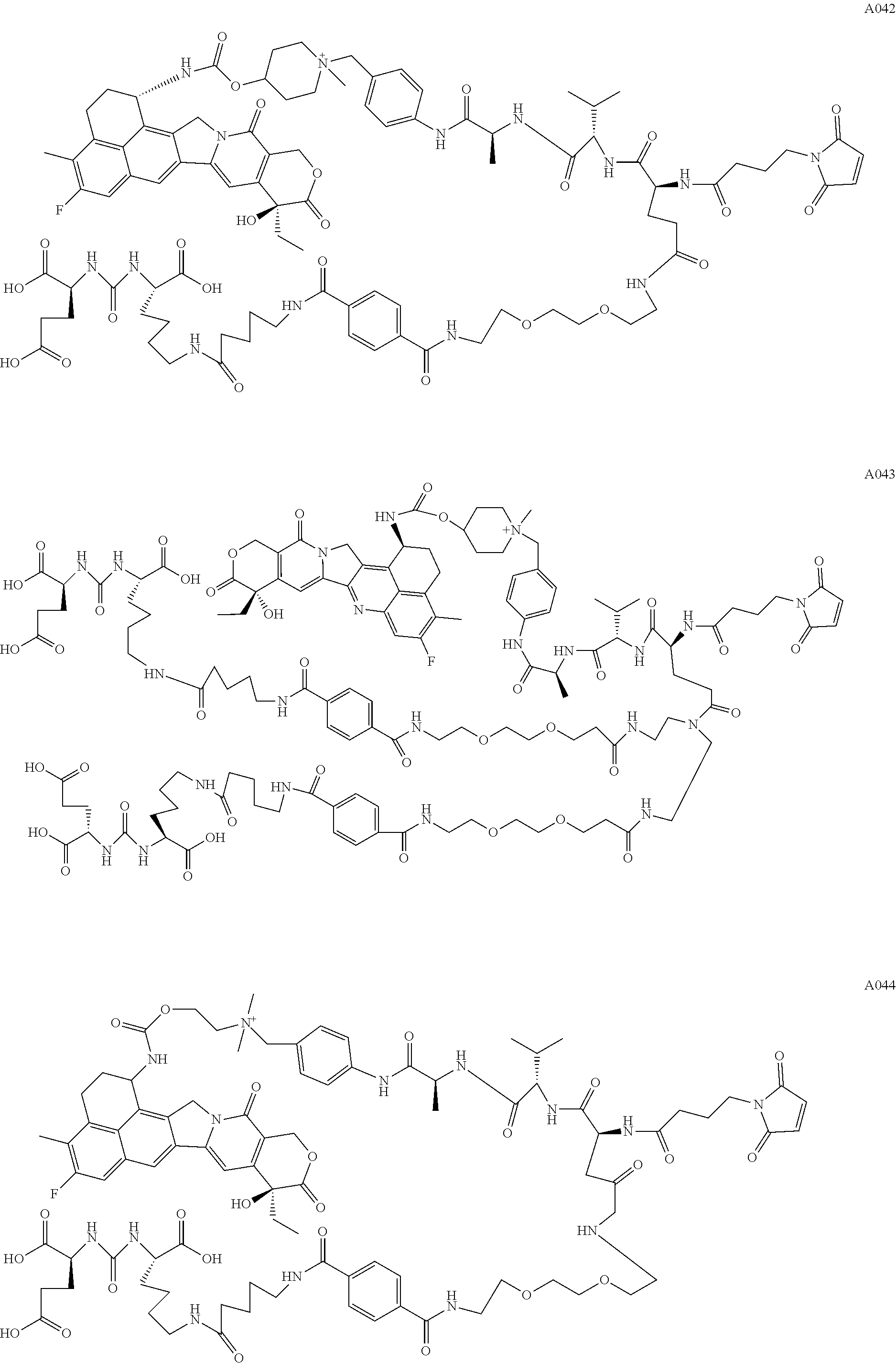

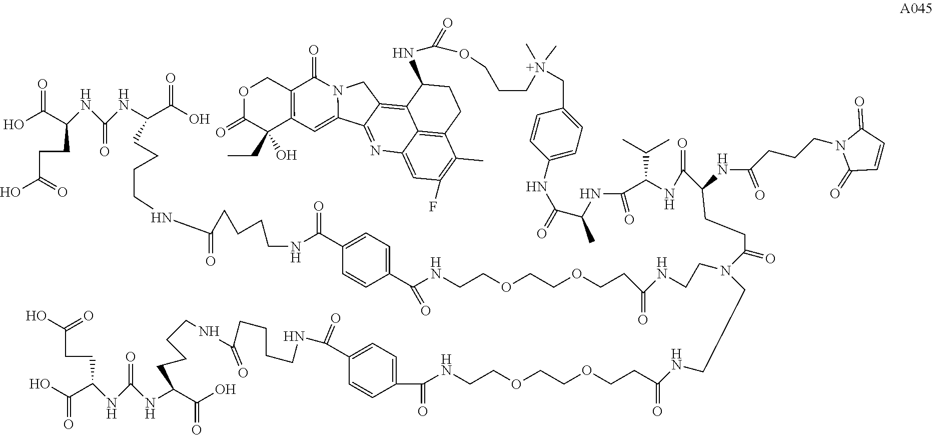 [SMILES: CC[C@@]1(O)C(=O)OCc2c1cc1n(c2=O)Cc2c-1nc1cc(F)c(C)c3c1c2[C@@H](NC(=O)OCCC[N+](C)(C)Cc1ccc(NC(=O)[C@H](C)NC(=O)[C@@H](NC(=O)[C@H](CCC(=O)N(CCNC(=O)CCOCCOCCNC(=O)c2ccc(C(=O)NCCCCC(=O)NCCCC[C@H](NC(=O)N[C@@H](CCC(=O)O)C(=O)O)C(=O)O)cc2)CCNC(=O)CCOCCOCCNC(=O)c2ccc(C(=O)NCCCCC(=O)NCCCC[C@H](NC(=O)N[C@@H](CCC(=O)O)C(=O)O)C(=O)O)cc2)NC(=O)CCCN2C(=O)C=CC2=O)C(C)C)cc1)CC3]

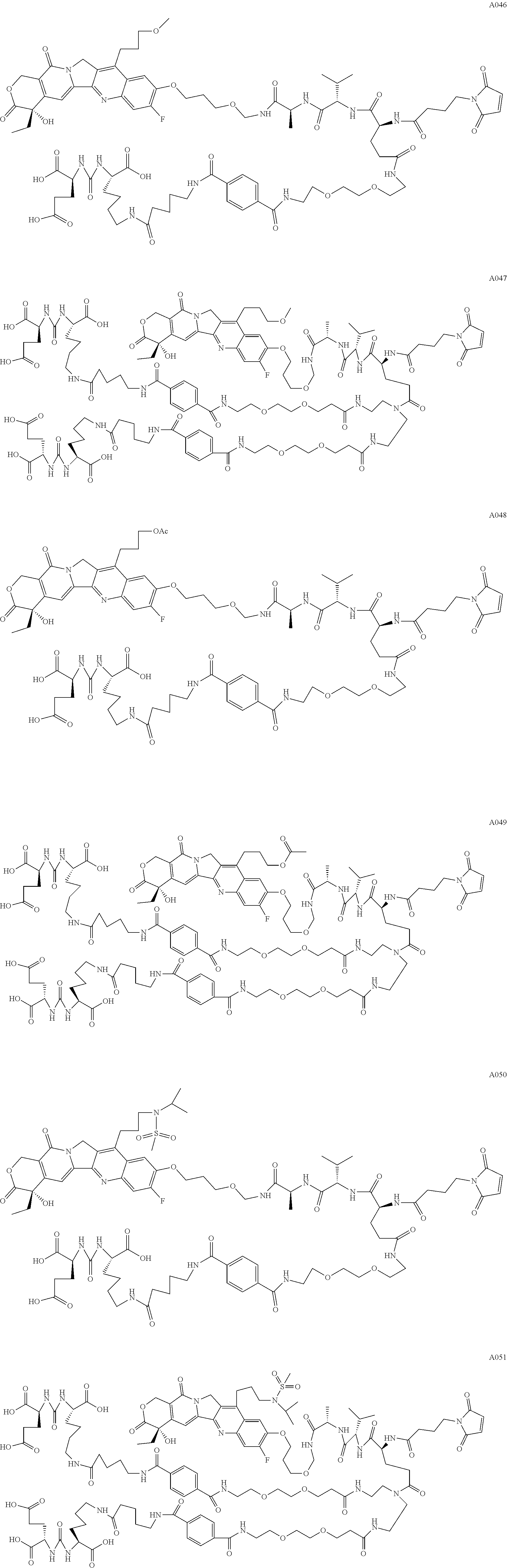

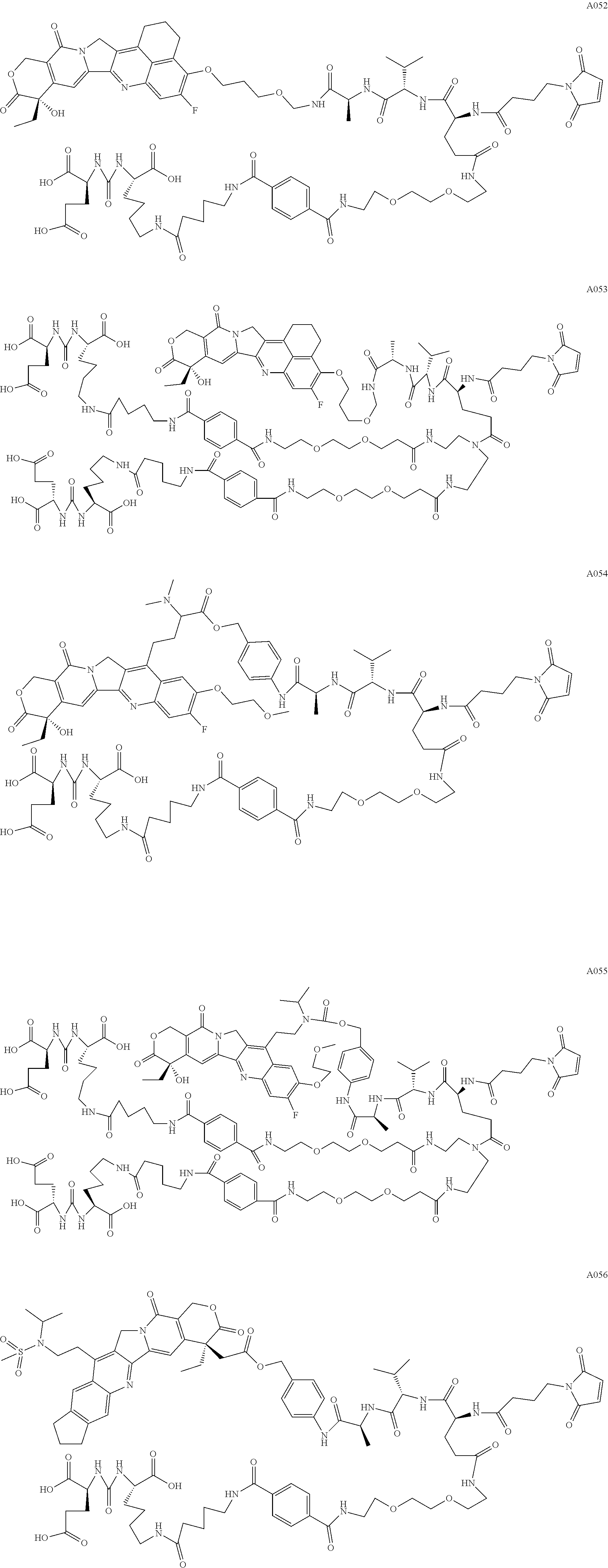

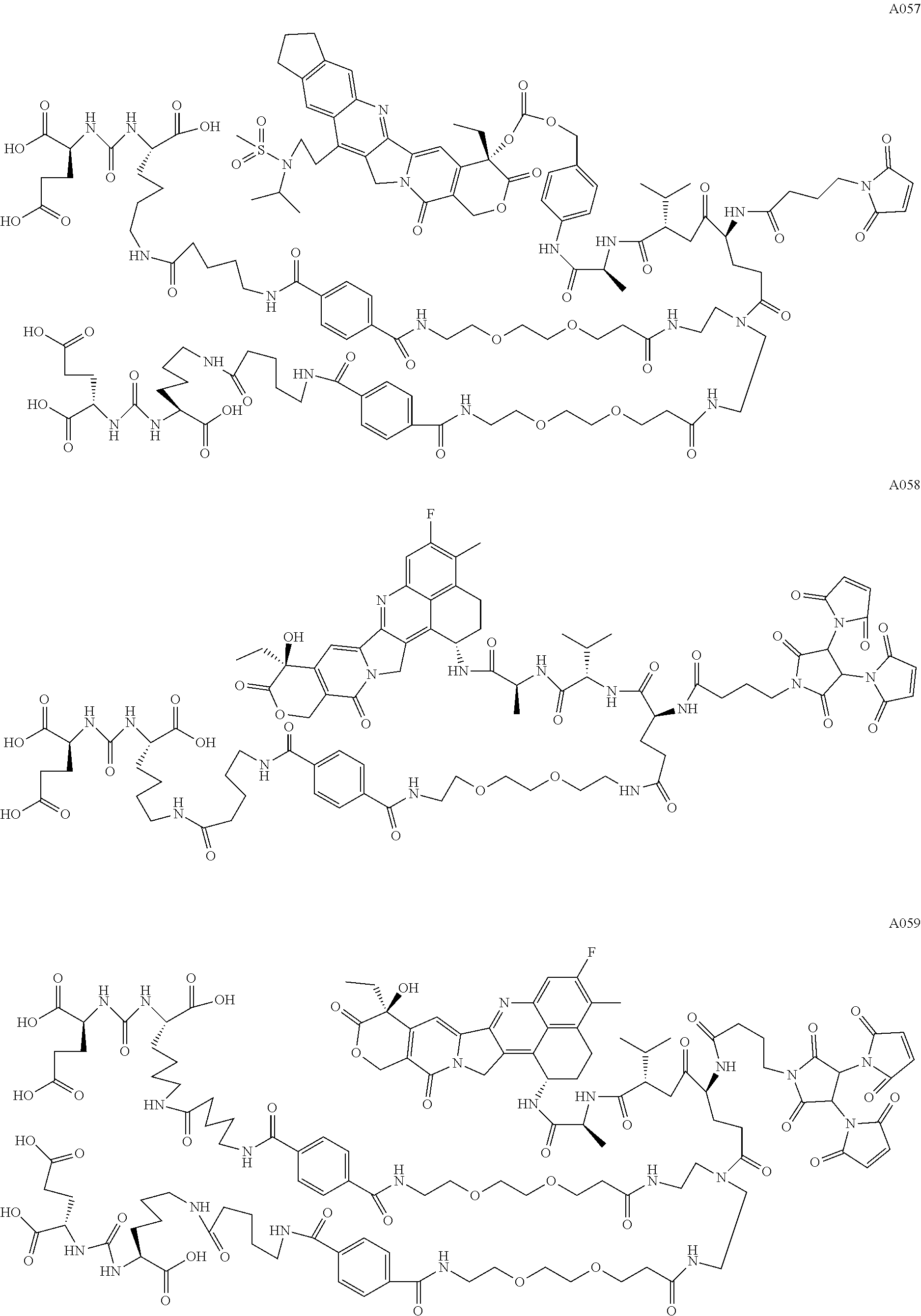

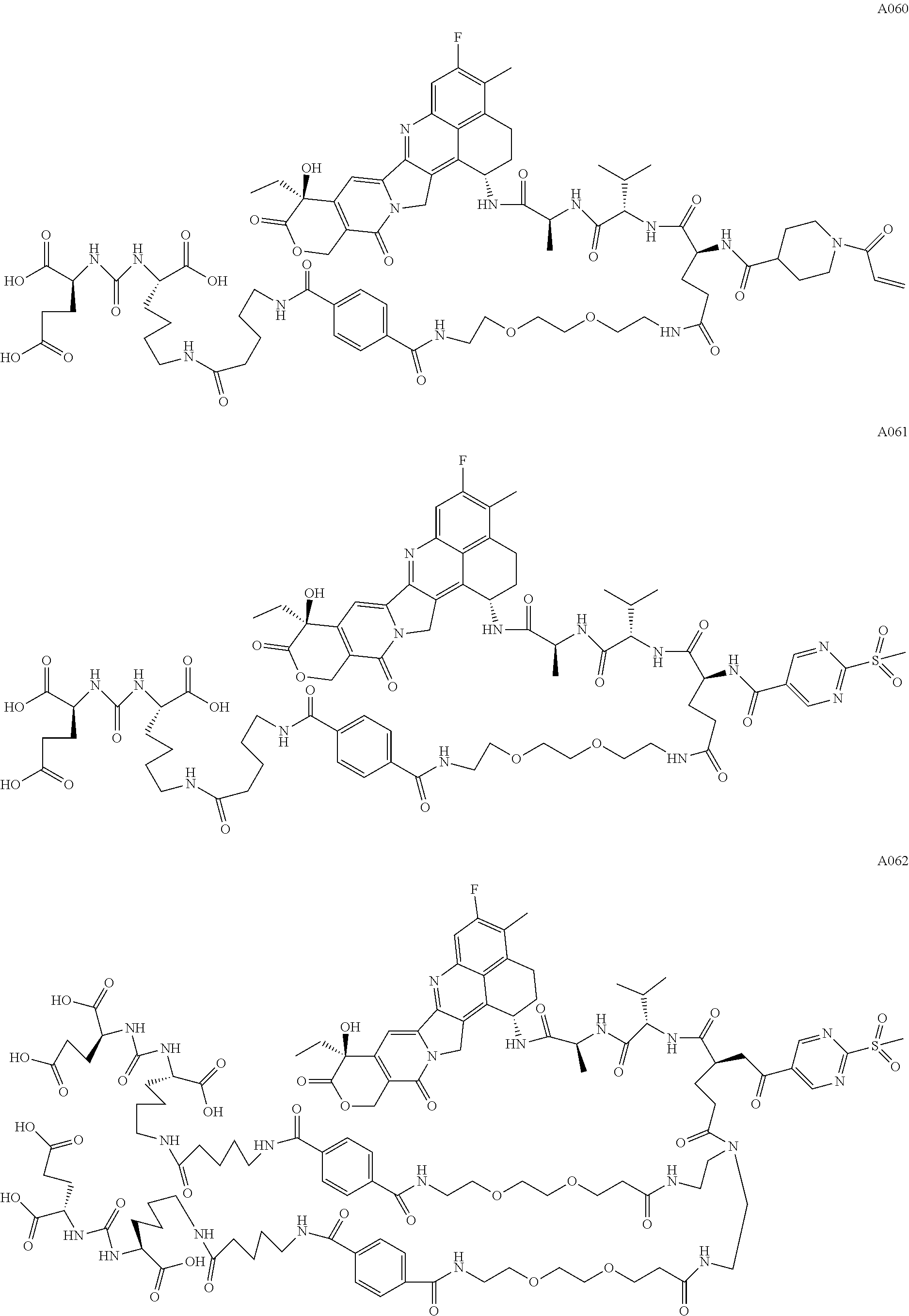

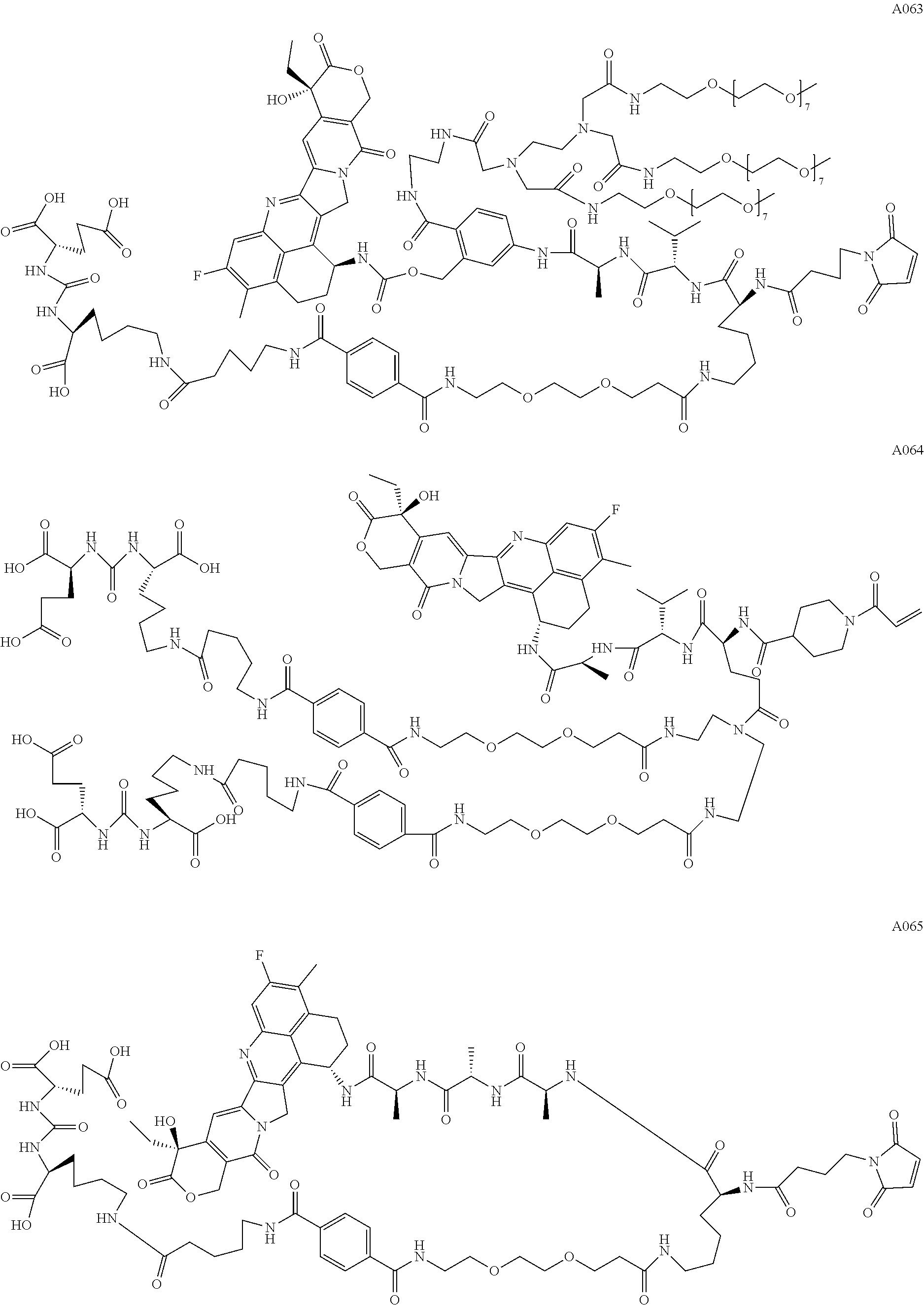

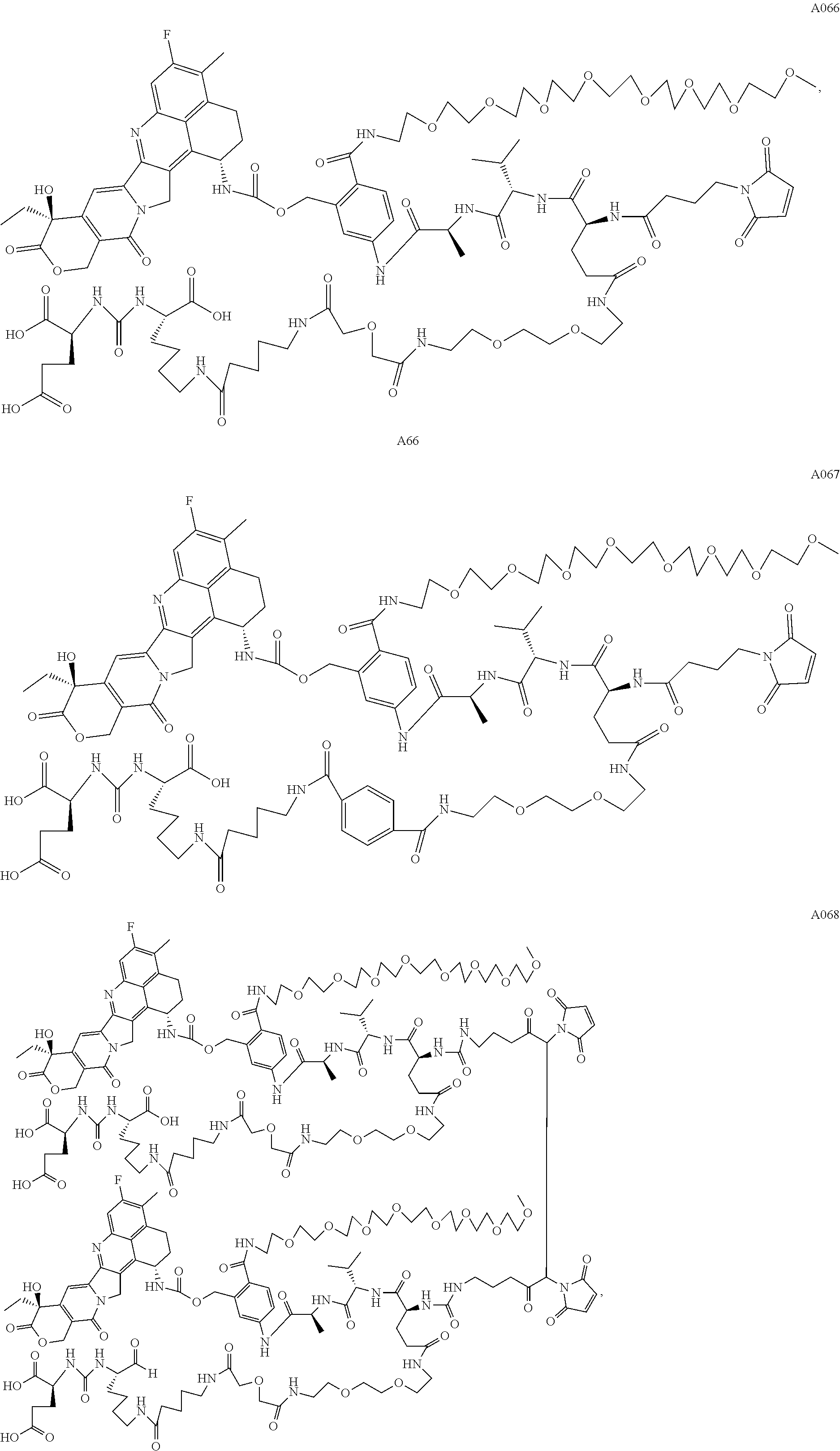

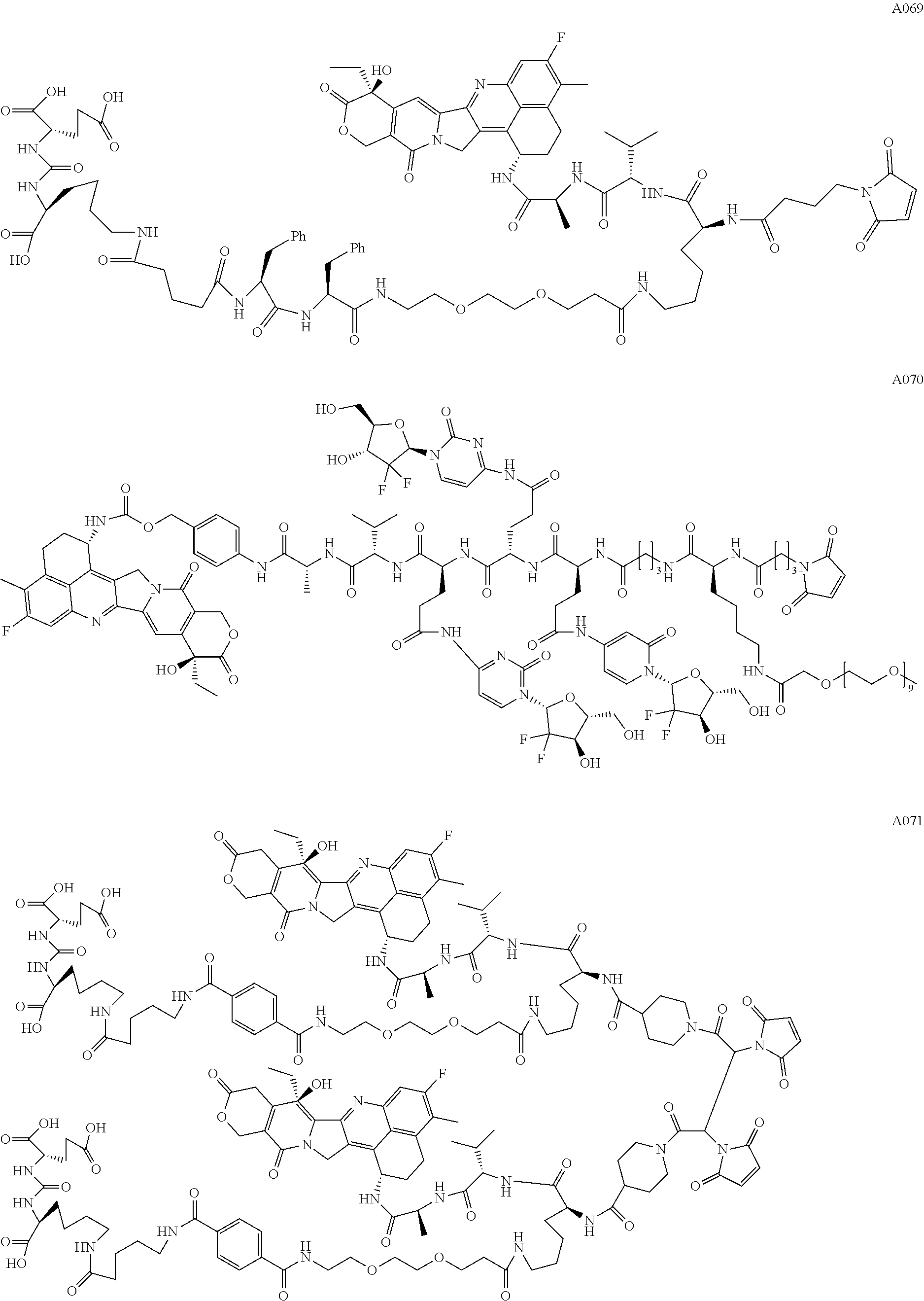

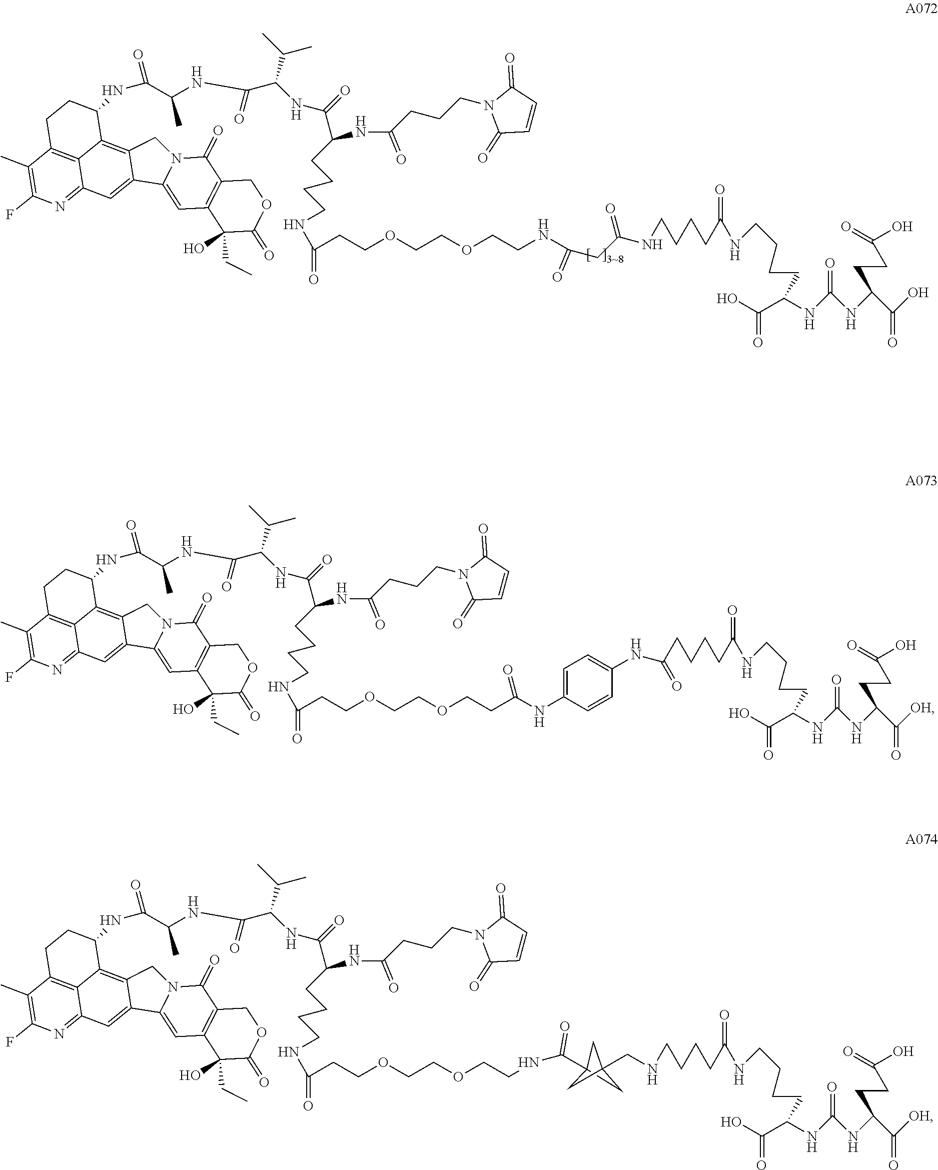

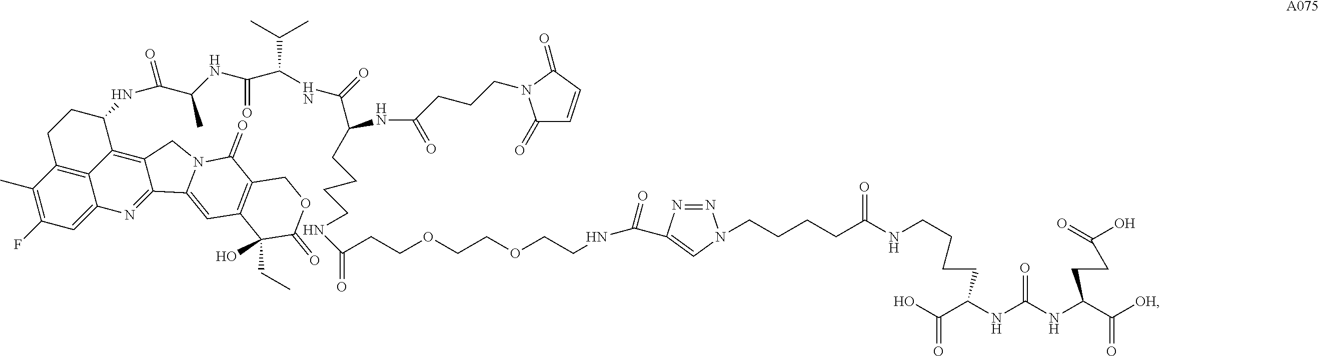

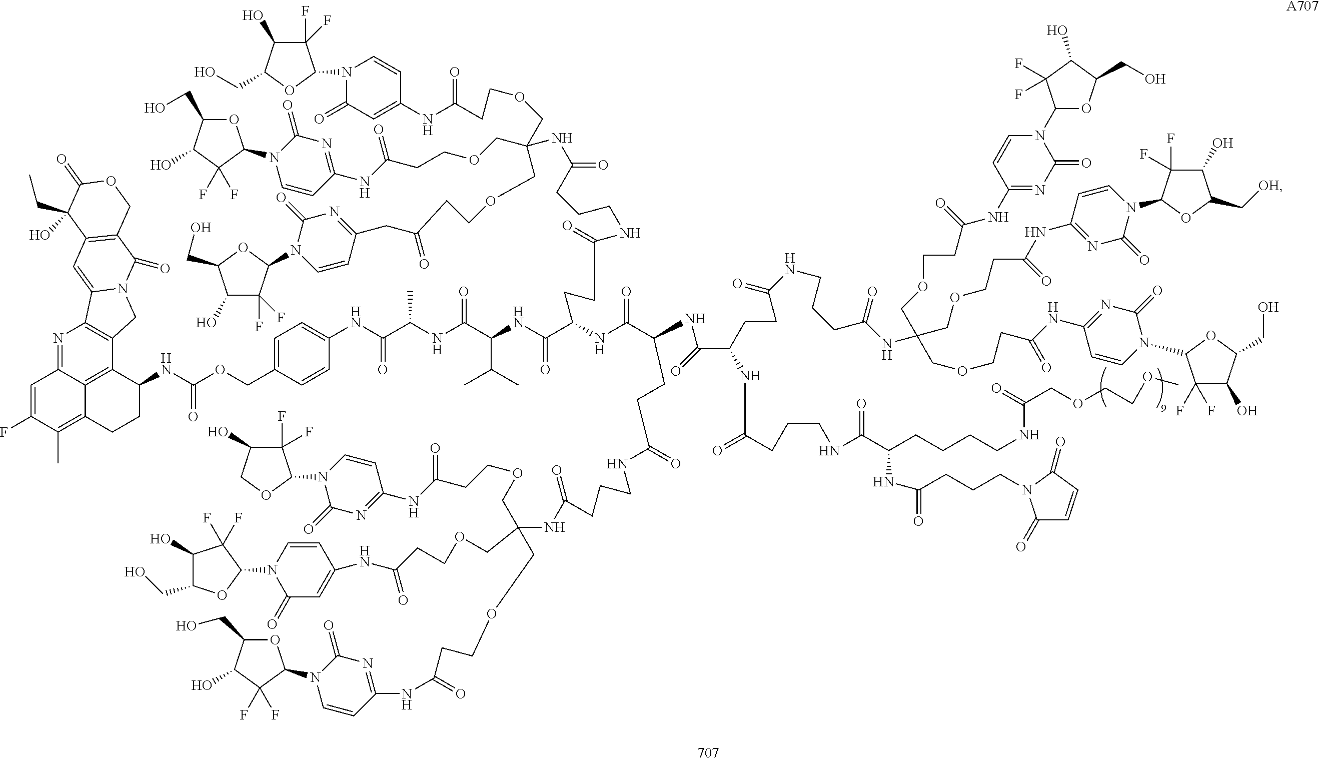

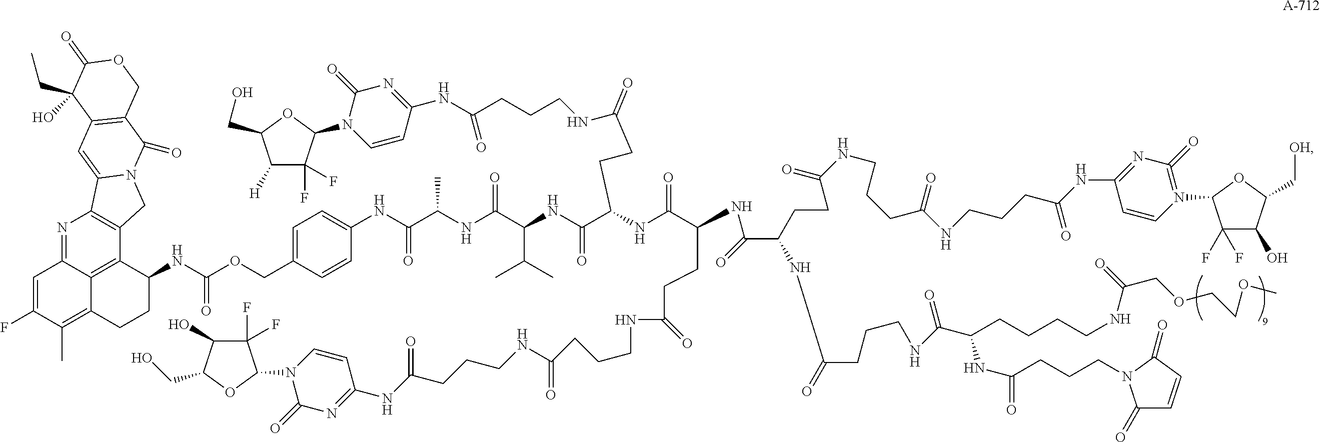

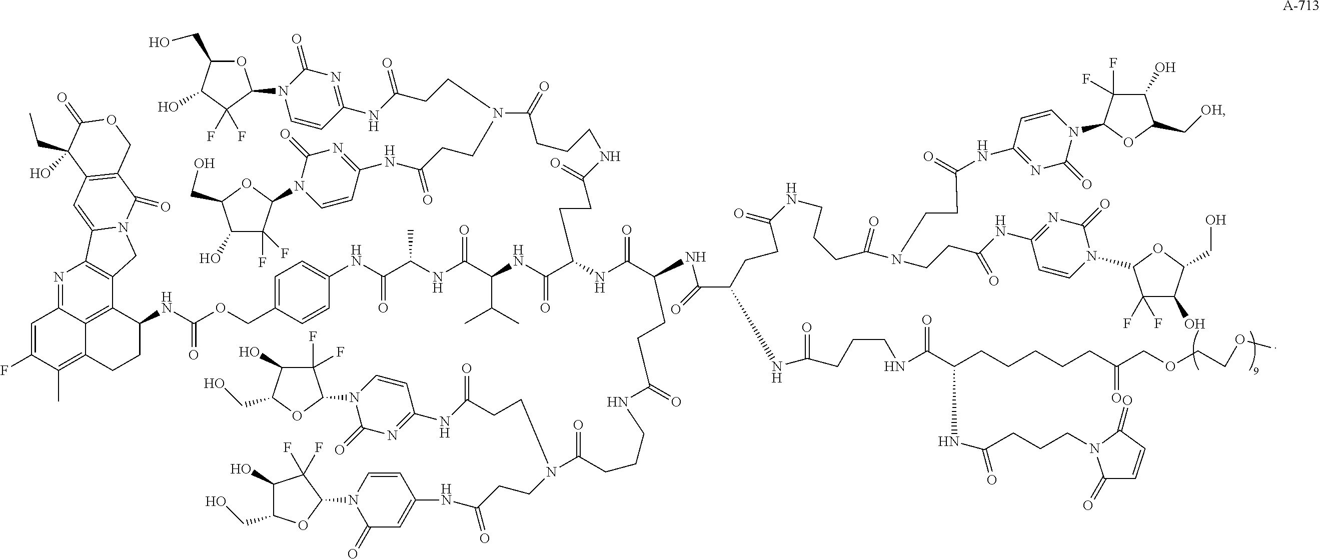

44. The antibody drug conjugate according to claim 1, wherein the antibody or the antibody like protein is selected from: one or several of a dAb, Fab, Fab′, F(ab′)₂, Fv, nanobody, diabody, triabody, tetrabody, miniantibody, a minibody, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, a probody, a probody fragment, small immune proteins (SIP), a lymphokine, a hormone, a vitamin, a growth factor, a colony stimulating factor, a nutrient-transport molecule, large molecular weight proteins, fusion proteins, kinase inhibitors, gene-targeting agents, nanoparticles or polymers modified with antibodies or large molecular weight proteins; a vitamin (including folate); or large molecular peptides, a polymeric micelle, a liposome, a lipoprotein-based drug carrier, a nano-particle drug carrier, a dendrimer, and a particle said above coating or linking with a cell-binding ligand or a protein.

45. The antibody drug conjugate according to claim 1, wherein the antibody or the antibody like protein is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any malfunctioned cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD13, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a,b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (trophoblastic glycoprotein, TPBG, 5T4, Wnt-activated inhibitory factor 1 or WAIF1), adenocarcinoma antigen, AGS-5, AGS-22M6, activin receptor-like kinase 1, AFP, AKAP-4, ALK, alpha integrin, alpha v beta6, amino-peptidase N, Amyloid beta, androgen receptor, angiopoietin 2, angiopoietin 3, annexin A1, anthrax toxin protective antigen, anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, carbonic anhydrase IX, cardiac myosin, CCL11 (C-C motif chemokine 11), CCR4 (C-C chemokine receptor type 4), CCR5, CD3E (epsilon), CEA (carcinoembryonic antigen), CEACAM3, CEACAM5 (carcino-embryonic antigen), CFD (Factor D), Ch4D5, cholecystokinin 2 (CCK2R), CLDN18 (Claudin-18), CLDN18.2 (Claudin-18.2), clumping factor A, cMet, CRIPTO, FCSFIR (colony stimulating factor 1 receptor), CSF2 (colony stimulating factor 2, granulocyte-macrophage colony-stimulating factor (GM-CSF)), CSP4, CTLA4 (cytotoxic T-lymphocyte-associated protein 4), CTAA16.88 tumor antigen, CXCR4, C-X-C chemokine receptor type 4, cyclic ADP ribose hydrolase, cyclin B1, CYP1B1, cytomegalovirus, cytomegalovirus glycoprotein B, Dabigatran, DLL3 (delta-like-ligand 3), DLL4 (delta-like-ligand 4), DPP4 (dipeptidyl-peptidase 4), DR5 (feath receptor 5), E. coli shiga toxin type-1, E. coli shiga toxin type-2, ED-B, EGFL7 (EGF-like domain-containing protein 7), EGFR, EGFRII, EGFRVIII, endoglin, endothelin B receptor, endotoxin, EpCAM (epithelial cell adhesion molecule), EphA2, Episialin, ERBB2 (epidermal growth factor receptor 2), ERBB3, ERG (TMPRSS2 ETS fusion gene), Escherichia coli, ETV6-AML, FAP (fibroblast activation protein alpha), FCGR1, alpha-Fetoprotein, Fibrin II, beta chain, fibronectin extra domain-B, FOLR (folate receptor), folate receptor alpha, folate hydrolase, Fos-related antigen 1F protein of respiratory syncytial virus, frizzled receptor, fucosyl GM1, GD2 ganglioside, G-28 (a cell surface antigen glyvolipid), GD3 idiotype, GloboH, glypican 3, N-glycolylneuraminic acid, GM3, GMCSF receptor α-chain, growth differentiation factor 8, GP100, GPNMB (trans-membrane glycoprotein NMB), GUCY2C (guanylate cyclase 2C, guanylyl cyclase C (GC-C), intestinal fuanylate cyclase, fuanylate cyclase-C receptor, heat-stable enterotoxin receptor (hSTAR)), heat shock proteins, hemagglutinin, hepatitis B surface antigen, hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, histone complex, HLA-DR (human leukocyte antigen), HLA-DR10, HLA-DRB, HMWMAA, human chorionic gonadotropin, HNGF, human scatter factor receptor kinase, HPV E6/E7, Hsp90, hTERT, ICAM-1 (Intercellular Adhesion Molecule 1), idiotype, IGFIR (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (insulin-like growth factor 2), Integrins (α4, α_{IIb}β₃, αvβ₃, α₄β₇, α₅β₁, α₆β₄, a₇β₇, αllβ₃, α₅β₅, αvβ₅), interferon gamma-induced protein, ITGA2, ITGB2, KIR2D, Kappa Ig, LCK, Le, Legumain, Lewis-Y antigen, LFA-1 (lymphocyte function-associated antigen 1, CD11a), LHRH, LINGO-1, lipoteichoic acid, LIVIA, LMP2, LTA, MAD-CT-1, MAD-CT-2, MAGE-1, MAGE-2, MAGE-3, MAGE A1, MAGE A3, MAGE 4, MART1, MCP-1, MIF (macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, myelin-associated glycoprotein, myostatin, NA17, NARP-1, NCA-90 (granulocyte antigen), Nectin-4 (ASG-22ME), NGF, neural apoptosis-regulated proteinase 1, NOGO-A, Notch receptor, nucleolin, neu oncogene product, NY-BR-1, NY-ESO-1, OX-40, OxLDL (oxidized low-density lipoprotein), OY-TES1, P21, p53 nonmutant, P97, Page 4, PAP, paratope of anti-(N-glycolylneuraminic acid), PAX3, PAX5, PCSK9, PDCD1 (PD-1, programmed cell death protein 1), PDGF-Ra (Alpha-type platelet-derived growth factor receptor), PDGFR-β, PDL-1, PLAC1, PLAP-like testicular alkaline phosphatase, platelet-derived growth factor receptor beta, phosphate-sodium co-transporter, PMEL 17, polysialic acid, proteinase3 (PR1), prostatic carcinoma, PS (Phosphatidylserine), prostatic carcinoma cells, Pseudomonas aeruginosa, PSMA, PSA, PSCA, rabies virus glycoprotein, RHD (Rh polypeptide 1 (RhPI)), Rhesus factor, RANKL, RhoC, Ras mutant, RGS5, ROBO4, respiratory syncytial virus, RON, ROR1, Sarcoma translocation breakpoints, SART3, sclerostin, SLAMF7 (SLAM family member 7), Selectin P, SDC1 (Syndecan 1), sLe (a), Somatomedin C, SIP (Sphingosine-1-phosphate), Somatostatin, sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (transforming growth factor beta), TGF-β1, TGF-β2 (transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (tumor necrosis apoptosis inducing ligand receptor 1), TRAILR2 (death receptor 5 (DR5)), tumor-associated calcium signal transducer 2, tumor specific glycosylation of MUC1, TWEAK receptor, TYRP1 (glycoprotein 75), TRP-1 (Trop-1), TRP-2 (Trop-2), tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

46. The antibody drug conjugate according to claim 45, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, or any cells that grow and divide at an unregulated, quickened pace to cause cancers.

47. The antibody drug conjugate according to claim 1, wherein the antibody drug conjugate is prepared homogenously via the following three key steps:
(1). incubating the antibody or antibody-like protein in the presence of an effective zinc (II) cation or zinc (II) cation-amino chelate/complex (Zn(NR₁R₂R₃)ₘ₁²⁺) and a reductant (Tris(2-carboxyethyl) phosphine (TCEP)) in a buffer system (including PBS, Mes, Bis-Tris, Bis-Tris Propane, Acetates, Histidine, Citrates, MES, or Borates) at pH 4.5˜8.5, 1˜10° C. for 1˜24 h to selectively reduce interchain disulfide bonds within the antibody, to generate thiols; wherein R₁, R₂ and R₃ in the formula of Zn(NR₁R₂R₃)ₘ₁²⁺ are independently selected from C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; m₁ is selected from 1, 2, 3, 4, 5, 6, 7 or 8; And (NR₁R₂R₃)ₘ₁ in Zn(NR₁R₂R₃)ₘ₁²⁺ can be form a dimer, trimer, tetramer, pentamer, or hexamer; and N, R₁, R₂ or R₃ groups can themselves or jointly form (or join together to form) 3˜10 member rings of heterocyclic, carbocyclic, diheterocyclic, or dicarbocyclic groups;
(2). introducing an effective amount of a cytotoxic drug-linker complex sequentially, to react with the thiol groups resulted from step (1) for 15 minutes to 4 hours; For the payload/linker complex containing a disulfide bond, the reduced antibody was isolated at 2-8° C. through a chromatography (with ion exchange or size exclusion column) or dialysis prior to addition of the cytotoxic drug-linker complex;
(3). adding effective amount of 4-(azidomethyl)-benzoic acid or p-azido-benzoic acid to quench excess reductants, and dehydroascorbic acid (DHAA) to re-oxidize unreacted thiol groups, and then purifying the resulted conjugates;
the step (3) can be replaced by: adding an effective amount of cystine to quench the excessive conjugation linker or linker/payload complex containing thiol reactive groups (maleimide); and adding 4-(azidomethyl)-benzoic acid or p-azido-benzoic acid or a disulfide compound (cystine) to quench the unreacted reductant (TCEP), simultaneously or sequentially, wherein addition of cystine to quench the unreacted reductant (TCEP) yields a cysteine which simultaneously quenches the excessive linker/payload complex, the yielded conjugate is optionally isolated by chromatography or UF/DF.

48. A pharmaceutical composition comprising a therapeutically effective amount of the antibody drug conjugate of claim 1, and a pharmaceutically acceptable salt, carrier, diluent, or excipient therefore, or a combination of the conjugates thereof, for use in the treatment or prevention of a cancer.

49. The pharmaceutical composition according to claim 48 is either in the liquid formula or in the formulated lyophilized solid, comprising by weight of: 0.01%-99% of one or more the antibody drug conjugate; 0.0%-20.0% of one or more polyols; 0.0%-2.0% of one or more surfactants; 0.0%-5.0% of one or more preservatives; 0.0%-30% of one or more amino acids; 0.0%-5.0% of one or more antioxidants; 0.0%-0.3% of one or more metal chelating agents; 0.0%-0.5% of hyaluronidase with activity of >500 u/mg; 0.0%-30.0% of one or more buffer salts for adjusting pH of the formulation to pH 4.5 to 7.5; and 0.0%-30.0% of one or more of isotonic agent for adjusting osmotic pressure between about 250 to 350 mOsm when reconstituted for administration to a patient;
wherein the polyol is selected from fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, or L-gluconate and its metallic salts;
wherein the surfactant is selected from polysorbate 20, polysorbate 40, polysorbate 65, polysorbate 80, polysorbate 81, or polysorbate 85, poloxamer, poly(ethylene oxide)-poly (propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is selected from benzyl alcohol, octadecyldimethylbenzyl ammonium chloride, hexamethonium chloride, benzalkonium chloride, benzethonium chloride, phenol, butyl and benzyl alcohol, alkyl parabens such as methyl or propyl paraben, catechol, resorcinol, cyclohexanol, 3-pentanol, or m-cresol;
wherein the amino acid is selected from arginine, cystine, glycine, lysine, histidine, ornithine, isoleucine, leucine, alanine, glycine glutamic acid or aspartic acid;
wherein the antioxidant is selected from ascorbic acid, glutathione, cystine or methionine;
wherein the chelating agent is selected from EDTA or EGTA;
wherein the buffer salt is selected from sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is selected from mannitol, sorbitol, sodium acetate, potassium chloride, sodium phosphate, potassium phosphate, trisodium citrate, or sodium chloride.

50. The pharmaceutical composition according to claim 48, is packed in a vial, bottle, pre-filled syringe, or pre-filled auto-injector syringe, in a form of a liquid or lyophilized solid.

51. The conjugate of claim 1, having in vitro, in vivo or ex vivo cell killing activity.

52. A pharmaceutical composition according to claim 48, wherein the pharmaceutical composition is administered concurrently with a chemotherapeutic agent, a radiation therapy, an immunotherapy agent, an autoimmune disorder agent, an anti-infectious agent or the other conjugates for use as a synergistical drug in treatment or prevention of a cancer.

53. The pharmaceutical composition according to claim 52, wherein the synergistical drug is selected from:
(1). a). an alkylating agent: selected from nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: including busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine];
b). A plant alkaloid: selected from the group consisting of Vinca alkaloids: including vincristine, vinblastine, vindesine, vinorelbine, and navelbin; Taxoids: comprising paclitaxel, docetaxol and their analogs, Maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c). A DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). An antimetabolite: selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (including mycophenolic acid, tiazofurin, ribavirin, EICAR); Ribonucleotide reductase Inhibitors: (including hydroxyurea, deferoxamine)]; [Pyrimidine analogs: Uracil analogs: (including ancitabine, azacitidine, 6-azauridine, capecitabine, carmofur, cytarabine, dideoxyuridine, doxifluridine, enocitabine, 5-Fluorouracil, floxuridine, ratitrexed); Cytosine analogs: (including cytarabine, cytosine arabinoside, fludarabine); Purine analogs: (including azathioprine, fludarabine, mercaptopurine, thiamiprine, thioguanine)]; folic acid replenisher, frolinic acid};
e). A hormonal therapy: selected from Receptor antagonists: [Anti-estrogen: (including megestrol, raloxifene, tamoxifen); LHRH agonists: (including goscrclin, leuprolide acetate); Anti-androgens: (including bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D₃ analogs: (including CB 1093, EB 1089 KH 1060, cholecalciferol, ergocalciferol); Photodynamic therapies: (including verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain);
f). A kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, Trastuzumab, Ranibizumab, Panitumumab, ispinesib;
g). A poly (ADP-ribose) polymerase (PARP) inhibitors selected from the group of olaparib, niraparib, iniparib, talazoparib, veliparib, CEP 9722 (Cephalon's), E7016 (Eisai's), BGB-290 (BeiGene's), or 3-aminobenzamide;
h). An antibiotic, selected from the group consisting of an enediyne antibiotic (selected from the group of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). A polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (including staurosporine), Actinomycins (including Actinomycin D, dactinomycin), amanitins, Bleomycins (including bleomycin A2, bleomycin B2, peplomycin), Anthracyclines (including daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((including Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, selected from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (including the group of T-2 toxin, verrucarin A, roridin A and anguidine); urethane, siRNA, antisense drugs;
(2). An anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3). An anti-infectious disease agents comprising:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem, cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefmetazole), oxacephem (flomoxef, latamoxef);
f). Glycopeptides: bleomycin, vancomycin (including oritavancin, telavancin), teicoplanin (dalbavancin), ramoplanin;
g). Glycylcyclines: tigecycline;
h). B-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). Anti-viral drugs comprising:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′,3′-dideoxynucleoside analogues (including the group consisting of3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of 8-1-thymidine and 8-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). pharmaceutically acceptable salts, acids, derivatives, hydrate or hydrated salt; or a crystalline structure; or an optical isomer, racemate, diastereomer or enantiomer of any of the above drugs.

54. The pharmaceutical composition according to claim 52, wherein the synergistical drug is selected from one or several of the following drugs: Abatacept, Abiraterone acetate, Abraxane, Acetaminophen/hydrocodone, Acalabrutinib, aducanumab, Adalimumab, ADXS31-142, ADXS-HER2, Afatinib dimaleate, Aldesleukin, Alectinib, Alemtuzumab, Alitretinoin, ado-trastuzumab emtansine, Amphetamine/dextroamphetamine, Anastrozole, Aripiprazole, anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, Crizotinib, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricitabine/Rilpivirine/Tenofovir, Disoproxil fumarate, Emtricitbine/tenofovir/efavirenz, Enoxaparin, Ensartinib, Enzalutamide, Epoetin alfa, erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, Iapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, Palbociclib, Palivizumab, Panitumumab, Panobinostat, Pazopanib, Pembrolizumab, PD-1 antibody, PD-L1 antibody, PD-1/VEGF antibody, PD-L1/VEGF antibody, Pemetrexed, Pertuzumab, Pneumococcal conjugate vaccine, Pomalidomide, Poziotinib, Pregabalin, ProscaVax, Propranolol, Quetiapine, Rabeprazole, Radium 223 chloride, Raloxifene, Raltegravir, Ramucirumab, Ranibizumab, Regorafenib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trastuzumab deruxtecan, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and their analogs, derivatives, pharmaceutically acceptable salts, carriers, diluents or excipients thereof or a combination above thereof.
